(19) United States
(12) Patent Application Publication
(10) Pub. No.: US 2024/0174667 A1
(43) Pub. Date: May 30, 2024

(54) AKT1 MODULATORS

(71) Applicant: Alterome Therapeutics, Inc., San Diego, CA (US)

(72) Inventors: Michael David BARTBERGER, Sherman Oaks, CA (US); Elena V. DNEPROVSKAIA, San Diego, CA (US); Yi FAN, San Diego, CA (US); Eric Anthony MURPHY, San Marcos, CA (US); Xuefeng ZHU, San Diego, CA (US)

(21) Appl. No.: 18/474,848

(22) Filed: Sep. 26, 2023

Related U.S. Application Data
(63) Continuation-in-part of application No. PCT/US2023/075058, filed on Sep. 25, 2023.
(60) Provisional application No. 63/582,697, filed on Sep. 14, 2023.
(60) Provisional application No. 63/580,327, filed on Sep. 1, 2023.
(60) Provisional application No. 63/508,418, filed on Jun. 15, 2023.
(60) Provisional application No. 63/498,770, filed on Apr. 27, 2023.
(60) Provisional application No. 63/377,183, filed on Sep. 26, 2022.
(60) Provisional application No. 63/582,697, filed on Sep. 14, 2023.
(60) Provisional application No. 63/580,327, filed on Sep. 1, 2023.
(60) Provisional application No. 63/508,418, filed on Jun. 15, 2023.
(60) Provisional application No. 63/498,770, filed on Apr. 27, 2023.
(60) Provisional application No. 63/377,183, filed on Sep. 26, 2022.

(51) Int. Cl. C07D 471/04; C07D 519/00

(52) U.S. Cl. CPC C07D 471/04; C07D 519/00; C07B 2200/05

(57) ABSTRACT

Provided herein are inhibitors of AKT1, pharmaceutical compositions comprising the inhibitory compounds, and methods for using the AKT1 inhibitory compounds for the treatment of disease.

### CROSS-REFERENCE TO RELATED APPLICATION

This application is a continuation in part of International Patent Application No. PCT/US2023/075058, filed on Sep. 25, 2023; which claims the benefit of U.S. Patent Application No. 63/377,183, filed on Sep. 26, 2022; U.S. Patent Application No. 63/498,770, filed on Apr. 27, 2023; U.S. Patent Application No. 63/508,418, filed on Jun. 15, 2023; U.S. Patent Application No. 63/580,327, filed on Sep. 1, 2023; and U.S. Patent Application No. 63/582,697, filed on Sep. 14, 2023, all of which are hereby incorporated by reference in their entirety.

### BACKGROUND

AKT is a protein kinase and mediates cell survival and proliferation by inhibiting pathways which promotes apoptosis. AKT signaling cascade dysfunction is observed in several cancer types and may be associated with tumor aggressiveness. Additionally, malfunction of AKT typically lead to enhanced proliferation, growth, survival, and resistance to apoptosis. Pharmaceutical agents with the ability to modulate AKT1 activity would be useful in the treatment of disease, such as cancer.

### BRIEF SUMMARY OF THE INVENTION

Provided herein are inhibitors of AKT1, pharmaceutical compositions comprising said inhibitory compounds, and methods for using said inhibitory compounds for the treatment of disease.

One embodiment provides a compound having the structure of Formula (I), or a pharmaceutically acceptable salt or solvate thereof:

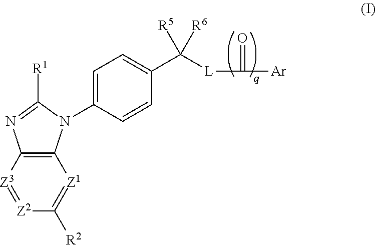

wherein:
   Z¹ is N, C—H, or C—R³;
   Z² is N, C—H, or C—R⁴;
   Z³ is N, C—OH, or C—R⁹;
   Ar is selected from:

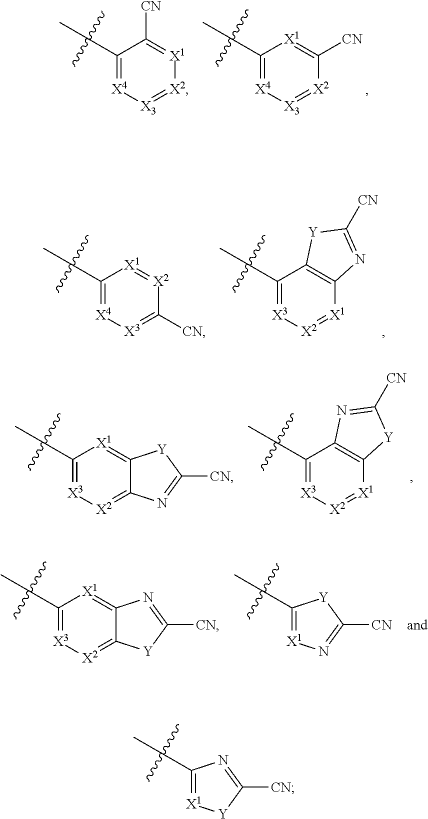

X² is N or C—R⁷;
X³ is N or C—R⁷;
X⁴ is N or C—R⁷;
Y is O, S, or N—R⁹;
R¹ is selected from hydrogen, optionally substituted C1-C6 alkyl, optionally substituted aryl, or optionally substituted heteroaryl;
R² is selected from hydrogen, halogen, —OH, —CN, —N(R⁹)₂, —OR⁹, —SR⁹, —SO₂R⁹, —CO₂R⁹, —CON(R⁹)₂, —SR⁹, —S(O)R⁹, —S(O)₂R⁹, optionally substituted C1-C6 alkyl, optionally substituted C1-C6 alkoxy, optionally substituted C2-C6 alkenyl, optionally substituted C2-C6 alkynyl, optionally substituted C3-C7 carbocyclyl, optionally substituted 4-membered to 6-membered heterocyclyl, optionally substituted aryl, aryl substituted with an optionally substituted 4-membered to 6-membered heterocyclyl, optionally substituted heteroaryl or heteroaryl substituted with an optionally substituted 3-membered to 6-membered carbocyclyl;
R³ is selected from optionally substituted C1-C6 alkyl, or optionally substituted aryl;
R⁴ is selected from halogen, —CN, optionally substituted C1-C6 alkyl, or optionally substituted aryl;
R⁵ and R⁶ are each independently hydrogen, deuterium, halogen, —OH, or optionally substituted C1-C6 alkyl; or R⁵ and R⁶ together form an oxo; or R⁵ and R⁶ join together to form a carbocycle or heterocycle;
each R⁷ is independently selected from hydrogen, deuterium, halogen, —OH, —SH, optionally substituted C1-C6 alkoxy, —S— (optionally substituted C1-C6 alkyl), —CN, optionally substituted C1-C6 alkyl, and optionally substituted aryl;
L is selected from —N(R⁸)—, or a divalent radical selected from.

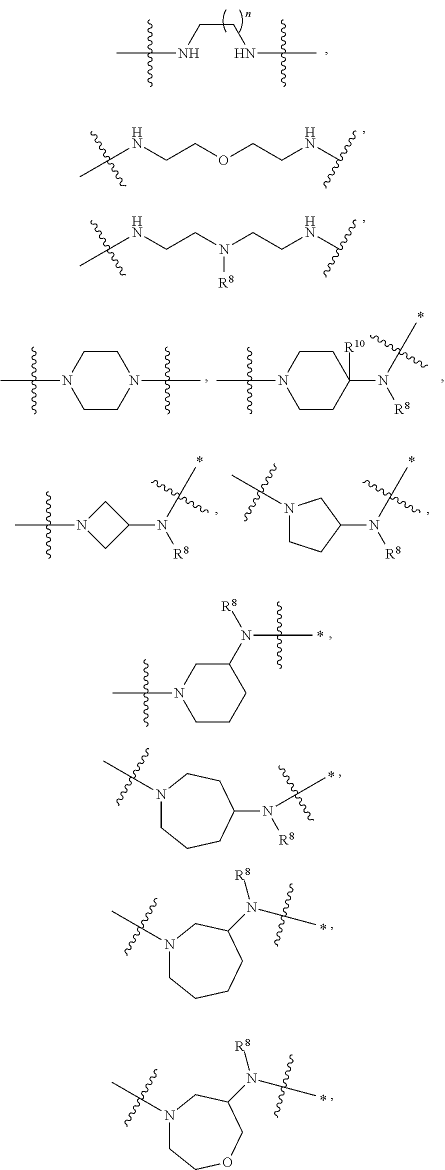

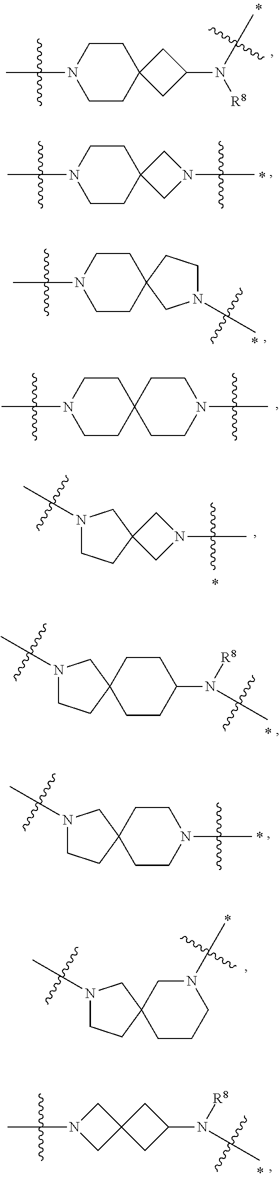

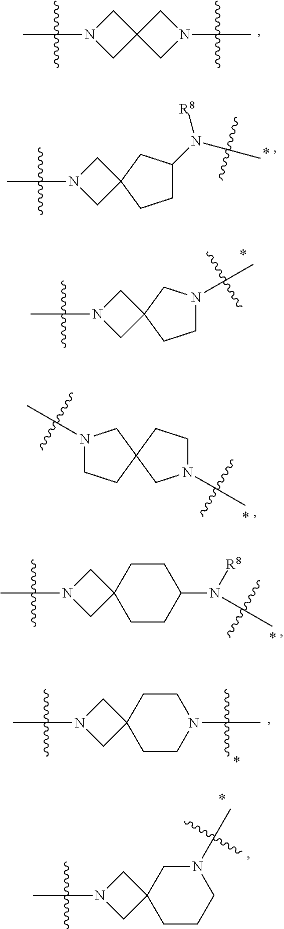

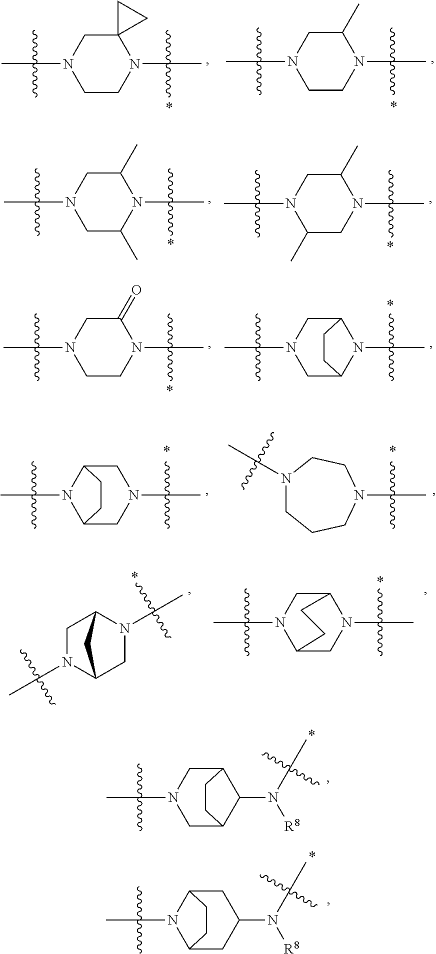

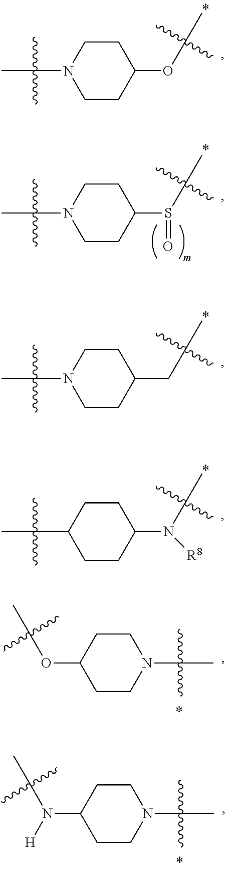

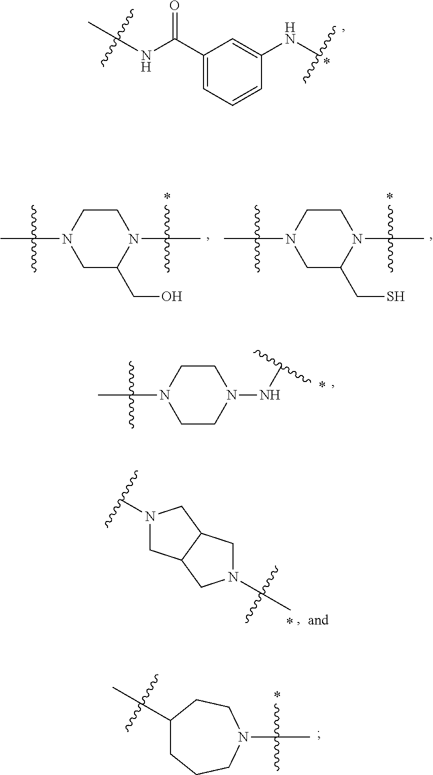 [SMILES: *NC(=O)c1cccc(N*)c1 ; *N1CCN(*)C(CO)C1 ; *N1CCN(*)C(CS)C1 ; *NN1CCN(*)CC1 ; *N1CC2CN(*)CC2C1 ; *C1CCCN(*)CC1]

wherein the asterisk (*) indicates the bond to the —CO—Ar group;
   R⁸ is hydrogen, optionally substituted C1-C6 alkyl, optionally substituted C3-C7 cycloalkyl, or optionally substituted heterocyclyl;
   each R⁹ is hydrogen, or optionally substituted C1-C6 alkyl;
   R¹⁰ is optionally substituted C1-C6 alkyl;
   m is 0, 1, or 2;
   n is 1, 2, or 3; and
   q is 0 or 1.

One embodiment provides a pharmaceutical composition comprising a compound of Formula (I), or pharmaceutically acceptable salt or solvate thereof, and at least one pharmaceutically acceptable excipient.

One embodiment provides a method of treating a disease or disorder in a patient in need thereof comprising administering to the patient a compound of Formula (I), or pharmaceutically acceptable salt or solvate thereof. Another embodiment provides the method wherein the disease or disorder is cancer.

### INCORPORATION BY REFERENCE

All publications, patents, and patent applications mentioned in this specification are herein incorporated by reference for the specific purposes identified herein.

### DETAILED DESCRIPTION OF THE INVENTION

As used herein and in the appended claims, the singular forms “a,” “and,” and “the” include plural referents unless the context clearly dictates otherwise. Thus, for example, reference to “an agent” includes a plurality of such agents, and reference to “the cell” includes reference to one or more cells (or to a plurality of cells) and equivalents thereof known to those skilled in the art, and so forth. When ranges are used herein for physical properties, such as molecular weight, or chemical properties, such as chemical formulae, all combinations and subcombinations of ranges and specific embodiments therein are intended to be included. The term “about” when referring to a number or a numerical range means that the number or numerical range referred to is an approximation within experimental variability (or within statistical experimental error), and thus the number or numerical range, in some instances, will vary between 1% and 15% of the stated number or numerical range. The term “comprising” (and related terms such as “comprise” or “comprises” or “having” or “including”) is not intended to exclude that in other certain embodiments, for example, an embodiment of any composition of matter, composition, method, or process, or the like, described herein, “consist of” or “consist essentially of” the described features.

### Definitions

As used in the specification and appended claims, unless specified to the contrary, the following terms have the meaning indicated below.

“Amino” refers to the —NH₂ radical.

“Cyano” refers to the —CN radical.

“Nitro” refers to the —NO₂ radical.

“Oxa” refers to the —O— radical.

“Oxo” refers to the ═O radical.

“Thioxo” refers to the ═S radical.

“Imino” refers to the ═N—H radical.

“Oximo” refers to the ═N—OH radical.

“Hydrazino” refers to the ═N—NH₂ radical.

“Alkyl” refers to a straight or branched hydrocarbon chain radical consisting solely of carbon and hydrogen atoms, containing no unsaturation, having from one to fifteen carbon atoms (e.g., C₁-C₁₅ alkyl). In certain embodiments, an alkyl comprises one to thirteen carbon atoms (e.g., C₁-C₁₃ alkyl). In certain embodiments, an alkyl comprises one to eight carbon atoms (e.g., C₁-C₈ alkyl). In other embodiments, an alkyl comprises one to five carbon atoms (e.g., C₁-C₅ alkyl). In other embodiments, an alkyl comprises one to four carbon atoms (e.g., C₁-C₄ alkyl). In other embodiments, an alkyl comprises one to three carbon atoms (e.g., C₁-C₃ alkyl). In other embodiments, an alkyl comprises one to two carbon atoms (e.g., C₁-C₂ alkyl). In other embodiments, an alkyl comprises one carbon atom (e.g., C₁ alkyl). In other embodiments, an alkyl comprises five to fifteen carbon atoms (e.g., C₅-C₁₅ alkyl). In other embodiments, an alkyl comprises five to eight carbon atoms (e.g., C₅-C₈ alkyl). In other embodiments, an alkyl comprises two to five carbon atoms (e.g., C₂-C₅ alkyl). In other embodiments, an alkyl comprises three to five carbon atoms (e.g., C₃-C₅ alkyl). In other embodiments, the alkyl group is selected from methyl, ethyl, 1-propyl (n-propyl), 1-methylethyl (iso-propyl), 1-butyl (n-butyl), 1-methylpropyl (sec-butyl), 2-methylpropyl (iso-butyl), 1,1-dimethylethyl (tert-butyl), 1-pentyl (n-pentyl). The alkyl is attached to the rest of the molecule by a single bond. Unless stated otherwise specifically in the specification, an alkyl group is optionally substituted by one or more of the following substituents: halo, cyano, nitro, oxo, thioxo, imino, oximo, trimethylsilanyl, —OW, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)R^{a} (where t is 1 or 2), —S(O)ₜOR^{a} (where t is 1 or 2), —S(O)ₜR^{a} (where t is 1 or 2) and —S(O)N(R′)₂ (where t is 1 or 2) where each R^{a} is independently hydrogen, alkyl (optionally substituted with halogen, hydroxy, methoxy, or trifluoromethyl), fluoroalkyl, carbocyclyl (optionally substituted with halogen, hydroxy, methoxy, or trifluoromethyl), carbocyclylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl). In certain embodiments, an optionally substituted alkyl is a haloalkyl. In other embodiments, an optionally substituted alkyl is a fluoroalkyl. In other embodiments, an optionally substituted alkyl is a —CF₃ group.

“Alkoxy” refers to a radical bonded through an oxygen atom of the formula —O-alkyl, where alkyl is an alkyl chain as defined above.

“Alkenyl” refers to a straight or branched hydrocarbon chain radical group consisting solely of carbon and hydrogen atoms, containing at least one carbon-carbon double bond, and having from two to twelve carbon atoms. In certain embodiments, an alkenyl comprises two to eight carbon atoms. In other embodiments, an alkenyl comprises two to four carbon atoms. The alkenyl is attached to the rest of the molecule by a single bond, for example, ethenyl (i.e., vinyl), prop-1-enyl (i.e., allyl), but-1-enyl, pent-1-enyl, penta-1,4-dienyl, and the like. Unless stated otherwise specifically in the specification, an alkenyl group is optionally substituted by one or more of the following substituents: halo, cyano, nitro, oxo, thioxo, imino, oximo, trimethylsilanyl, —OR^{a}, —SR, —OC(O)—R, —N(R′)₂, —C(O)R, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR^{a} (where t is 1 or 2), —S(O)OR^{a} (where t is 1 or 2), —S(O)ₜR^{a} (where t is 1 or 2) and —S(O)N(R^{a})₂ (where t is 1 or 2) where each R^{a} is independently hydrogen, alkyl (optionally substituted with halogen, hydroxy, methoxy, or trifluoromethyl), fluoroalkyl, carbocyclyl (optionally substituted with halogen, hydroxy, methoxy, or trifluoromethyl), carbocyclylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl).

“Alkynyl” refers to a straight or branched hydrocarbon chain radical group consisting solely of carbon and hydrogen atoms, containing at least one carbon-carbon triple bond, having from two to twelve carbon atoms. In certain embodiments, an alkynyl comprises two to eight carbon atoms. In other embodiments, an alkynyl comprises two to six carbon atoms. In other embodiments, an alkynyl comprises two to four carbon atoms. The alkynyl is attached to the rest of the molecule by a single bond, for example, ethynyl, propynyl, butynyl, pentynyl, hexynyl, and the like. Unless stated otherwise specifically in the specification, an alkynyl group is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R)C(O)OR^{a}, —OC(O)—N(R^{a})₂, —N(R^{a})C(O)R^{a}, —N(R^{a})S(O)NR^{a} (where t is 1 or 2), —S(O)ₜOR^{a} (where t is 1 or 2), —S(O)NR^{a} (where t is 1 or 2) and —S(O)N(R^{a})₂ (where t is 1 or 2) where each R^{a} is independently hydrogen, alkyl (optionally substituted with halogen, hydroxy, methoxy, or trifluoromethyl), fluoroalkyl, carbocyclyl (optionally substituted with halogen, hydroxy, methoxy, or trifluoromethyl), carbocyclylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl).

“Alkylene” or “alkylene chain” refers to a straight or branched divalent hydrocarbon chain linking the rest of the molecule to a radical group, consisting solely of carbon and hydrogen, containing no unsaturation, and having from one to twelve carbon atoms, for example, methylene, ethylene, propylene, n-butylene, and the like. The alkylene chain is attached to the rest of the molecule through a single bond and to the radical group through a single bond. The points of attachment of the alkylene chain to the rest of the molecule and to the radical group are through one carbon in the alkylene chain or through any two carbons within the chain. In certain embodiments, an alkylene comprises one to eight carbon atoms (e.g., C₁-C₈ alkylene). In other embodiments, an alkylene comprises one to five carbon atoms (e.g., C₁-C₅ alkylene). In other embodiments, an alkylene comprises one to four carbon atoms (e.g., C₁-C₄ alkylene). In other embodiments, an alkylene comprises one to three carbon atoms (e.g., C₁-C₃ alkylene). In other embodiments, an alkylene comprises one to two carbon atoms (e.g., C₁-C₂ alkylene). In other embodiments, an alkylene comprises one carbon atom (e.g., C₁ alkylene). In other embodiments, an alkylene comprises five to eight carbon atoms (e.g., C₅-C₈ alkylene). In other embodiments, an alkylene comprises two to five carbon atoms (e.g., C₂-C₅ alkylene). In other embodiments, an alkylene comprises three to five carbon atoms (e.g., C₃-C₅ alkylene). Unless stated otherwise specifically in the specification, an alkylene chain is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)NR^{a} (where t is 1 or 2), —S(O)OR^{a} (where t is 1 or 2), —S(O)NR^{a} (where t is 1 or 2) and —S(O)N(R^{a})₂ (where t is 1 or 2) where each R^{a} is independently hydrogen, alkyl (optionally substituted with halogen, hydroxy, methoxy, or trifluoromethyl), fluoroalkyl, carbocyclyl (optionally substituted with halogen, hydroxy, methoxy, or trifluoromethyl), carbocyclylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl).

“Alkenylene” or “alkenylene chain” refers to a straight or branched divalent hydrocarbon chain linking the rest of the molecule to a radical group, consisting solely of carbon and hydrogen, containing at least one carbon-carbon double bond, and having from two to twelve carbon atoms. The alkenylene chain is attached to the rest of the molecule through a single bond and to the radical group through a single bond. In certain embodiments, an alkenylene comprises two to eight carbon atoms (e.g., C₂-C₅ alkenylene). In other embodiments, an alkenylene comprises two to five carbon atoms (e.g., C₂-C₅ alkenylene). In other embodiments, an alkenylene comprises two to four carbon atoms (e.g., C₂-C₄ alkenylene). In other embodiments, an alkenylene comprises two to three carbon atoms (e.g., C₂-C₃ alkenylene). In other embodiments, an alkenylene comprises two carbon atoms (e.g., C₂ alkenylene). In other embodiments, an alkenylene comprises five to eight carbon atoms (e.g., C₅-C₈alkenylene). In other embodiments, an alkenylene comprises three to five carbon atoms (e.g., C₃-C₅ alkenylene). Unless stated otherwise specifically in the specification, an alkenylene chain is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR^{a} (where t is 1 or 2), —S(O)ₜR^{a} (where t is 1 or 2), —S(O)NR^{a} (where t is 1 or 2) and —S(O)ₜN(R^{a})₂ (where t is 1 or 2) where each R¹ is independently hydrogen, alkyl (optionally substituted with halogen, hydroxy, methoxy, or trifluoromethyl), fluoroalkyl, carbocyclyl (optionally substituted with halogen, hydroxy, methoxy, or trifluoromethyl), carbocyclylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl).

“Alkynylene” or “alkynylene chain” refers to a straight or branched divalent hydrocarbon chain linking the rest of the molecule to a radical group, consisting solely of carbon and hydrogen, containing at least one carbon-carbon triple bond, and having from two to twelve carbon atoms. The alkynylene chain is attached to the rest of the molecule through a single bond and to the radical group through a single bond. In certain embodiments, an alkynylene comprises two to eight carbon atoms (e.g., C₂-C₈ alkynylene). In other embodiments, an alkynylene comprises two to five carbon atoms (e.g., C₂-C₅ alkynylene). In other embodiments, an alkynylene comprises two to four carbon atoms (e.g., C₂-C₄ alkynylene). In other embodiments, an alkynylene comprises two to three carbon atoms (e.g., C₂-C₃ alkynylene). In other embodiments, an alkynylene comprises two carbon atoms (e.g., C₂ alkynylene). In other embodiments, an alkynylene comprises five to eight carbon atoms (e.g., C₅-C₈alkynylene). In other embodiments, an alkynylene comprises three to five carbon atoms (e.g., C₃-C₅ alkynylene). Unless stated otherwise specifically in the specification, an alkynylene chain is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R′)₂, —N(R′)C(O)OR^{a}, —OC(O)—N(R^{a})₂, —N(R^{a})C(O)R^{a}, —N(R^{a})S(O)R^{a} (where t is 1 or 2), —S(O)ₜR^{a} (where t is 1 or 2), —S(O)R^{a} (where t is 1 or 2) and —S(O)N(R^{a})₂ (where t is 1 or 2) where each R⁷ is independently hydrogen, alkyl (optionally substituted with halogen, hydroxy, methoxy, or trifluoromethyl), fluoroalkyl, carbocyclyl (optionally substituted with halogen, hydroxy, methoxy, or trifluoromethyl), carbocyclylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl).

“Aryl” refers to a radical derived from an aromatic monocyclic or multicyclic hydrocarbon ring system by removing a hydrogen atom from a ring carbon atom. The aromatic monocyclic or multicyclic hydrocarbon ring system contains only hydrogen and carbon from five to eighteen carbon atoms, where at least one of the rings in the ring system is fully unsaturated, i.e., it contains a cyclic, delocalized (4n+2) π-electron system in accordance with the Hückel theory. The ring system from which aryl groups are derived include, but are not limited to, groups such as benzene, fluorene, indane, indene, tetralin and naphthalene. Unless stated otherwise specifically in the specification, the term “aryl” or the prefix “ar-” (such as in “aralkyl”) is meant to include aryl radicals optionally substituted by one or more substituents independently selected from optionally substituted alkyl, optionally substituted alkenyl, optionally substituted alkynyl, halo, cyano, nitro, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(OR^{a} (where t is 1 or 2), —R^{b}—S(O)ₜR^{a} (where t is 1 or 2), —R^{b}—S(O)OR^{a} (where t is 1 or 2) and —R^{b}—S(O)N(R^{a})₂ (where t is 1 or 2), where each R^{a} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl), each R^{b} is independently a direct bond or a straight or branched alkylene or alkenylene chain, and R^{c} is a straight or branched alkylene or alkenylene chain, and where each of the R^{a}, R^{b}, or R^{c} substituents is unsubstituted unless otherwise indicated.

“Aralkyl” refers to a radical of the formula —R^{c}-aryl where R is an alkylene chain as defined above, for example, methylene, ethylene, and the like. The alkylene chain part of the aralkyl radical is optionally substituted as described above for an alkylene chain. The aryl part of the aralkyl radical is optionally substituted as described above for an aryl group.

“Aralkenyl” refers to a radical of the formula —R^{d}-aryl where R^{d} is an alkenylene chain as defined above. The aryl part of the aralkenyl radical is optionally substituted as described above for an aryl group. The alkenylene chain part of the aralkenyl radical is optionally substituted as defined above for an alkenylene group.

“Aralkynyl” refers to a radical of the formula —R-aryl, where R^{c} is an alkynylene chain as defined above. The aryl part of the aralkynyl radical is optionally substituted as described above for an aryl group. The alkynylene chain part of the aralkynyl radical is optionally substituted as defined above for an alkynylene chain.

“Aralkoxy” refers to a radical bonded through an oxygen atom of the formula —O—R^{c}-aryl where R^{c} is an alkylene chain as defined above, for example, methylene, ethylene, and the like. The alkylene chain part of the aralkyl radical is optionally substituted as described above for an alkylene chain. The aryl part of the aralkyl radical is optionally substituted as described above for an aryl group.

“Carbocyclyl” refers to a stable non-aromatic monocyclic or polycyclic hydrocarbon radical consisting solely of carbon and hydrogen atoms, which includes fused or bridged ring systems, having from three to fifteen carbon atoms. In certain embodiments, a carbocyclyl comprises three to ten carbon atoms. In other embodiments, a carbocyclyl comprises five to seven carbon atoms. The carbocyclyl is attached to the rest of the molecule by a single bond. Carbocyclyl is saturated (i.e., containing single C—C bonds only) or unsaturated (i.e., containing one or more double bonds or triple bonds). A fully saturated carbocyclyl radical is also referred to as “cycloalkyl.” Examples of monocyclic cycloalkyls include, e.g., cyclopropyl, cyclobutyl, cyclopentyl, cyclohexyl, cycloheptyl, and cyclooctyl. An unsaturated carbocyclyl is also referred to as “cycloalkenyl.” Examples of monocyclic cycloalkenyls include, e.g., cyclopentenyl, cyclohexenyl, cycloheptenyl, and cyclooctenyl. Polycyclic carbocyclyl radicals include, for example, adamantyl, norbornyl (i.e., bicyclo[2.2.1]heptanyl), norbornenyl, decalinyl, 7,7-dimethyl-bicyclo[2.2]heptanyl, and the like. Unless otherwise stated specifically in the specification, the term “carbocyclyl” is meant to include carbocyclyl radicals that are optionally substituted by one or more substituents independently selected from optionally substituted alkyl, optionally substituted alkenyl, optionally substituted alkynyl, halo, oxo, thioxo, cyano, nitro, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)R^{a} (where t is 1 or 2), —R^{b}—S(O)R^{a} (where t is 1 or 2), —R^{b}—S(O)ₜR^{a} (where t is 1 or 2) and —R^{b}—S(O)N(R^{a})₂ (where t is 1 or 2), where each R^{a} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl), each R^{b} is independently a direct bond or a straight or branched alkylene or alkenylene chain, and R^{c} is a straight or branched alkylene or alkenylene chain, and where each of the R^{a}, R^{b}, or R^{c} substituents is unsubstituted unless otherwise indicated.

“Carbocyclylalkyl” refers to a radical of the formula —R^{c}-carbocyclyl where R^{c} is an alkylene chain as defined above. The alkylene chain and the carbocyclyl radical is optionally substituted as defined above.

“Carbocyclylalkynyl” refers to a radical of the formula —R^{c}-carbocyclyl where R^{c} is an alkynylene chain as defined above. The alkynylene chain and the carbocyclyl radical is optionally substituted as defined above.

“Carbocyclylalkoxy” refers to a radical bonded through an oxygen atom of the formula —O—R^{c}-carbocyclyl where R^{c} is an alkylene chain as defined above. The alkylene chain and the carbocyclyl radical is optionally substituted as defined above.

“Halo” or “halogen” refers to bromo, chloro, fluoro or iodo substituents.

“Fluoroalkyl” refers to an alkyl radical, as defined above, that is substituted by one or more fluoro radicals, as defined above, for example, trifluoromethyl, difluoromethyl, fluoromethyl, 2,2,2-trifluoroethyl, 1-fluoromethyl-2-fluoroethyl, and the like. In some embodiments, the alkyl part of the fluoroalkyl radical is optionally substituted as defined above for an alkyl group.

“Heterocyclyl” refers to a stable 3- to 18-membered non-aromatic ring radical that comprises two to twelve carbon atoms and from one to six heteroatoms selected from nitrogen, oxygen and sulfur. Unless stated otherwise specifically in the specification, the heterocyclyl radical is a monocyclic, bicyclic, tricyclic, or tetracyclic ring system, which optionally includes fused or bridged ring systems. The heteroatoms in the heterocyclyl radical are optionally oxidized. One or more nitrogen atoms, if present, are optionally quaternized. The heterocyclyl radical is partially or fully saturated. The heterocyclyl is attached to the rest of the molecule through any atom of the ring(s). Examples of such heterocyclyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. Unless stated otherwise specifically in the specification, the term “heterocyclyl” is meant to include heterocyclyl radicals as defined above that are optionally substituted by one or more substituents selected from optionally substituted alkyl, optionally substituted alkenyl, optionally substituted alkynyl, halo, fluoroalkyl, oxo, thioxo, cyano, nitro, —R^{b}—OR^{a}3, —R^{b}—OC(O)—R^{a}, —R^{b}—OC(O)—OR^{a}, —R^{b}—OC(O)—N(R³)₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R—N(R′)S(O)NR^{a} (where t is 1 or 2), —R^{b}—S(O)ₜR^{a} (where t is 1 or 2), —R^{b}—S(O)ₜR^{a} (where t is 1 or 2) and —R^{b}—S(O)N(R^{a})₂ (where t is 1 or 2), where each R¹ is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl), each R^{b} is independently a direct bond or a straight or branched alkylene or alkenylene chain, and R^{c} is a straight or branched alkylene or alkenylene chain, and where each of the R^{a}, R^{b}, or R^{c} substituents is unsubstituted unless otherwise indicated.

“N-heterocyclyl” or “N-attached heterocyclyl” refers to a heterocyclyl radical as defined above containing at least one nitrogen and where the point of attachment of the heterocyclyl radical to the rest of the molecule is through a nitrogen atom in the heterocyclyl radical. An N-heterocyclyl radical is optionally substituted as described above for heterocyclyl radicals. Examples of such N-heterocyclyl radicals include, but are not limited to, 1-morpholinyl, 1-piperidinyl, 1-piperazinyl, 1-pyrrolidinyl, pyrazolidinyl, and imidazolidinyl.

“C-heterocyclyl” or “C-attached heterocyclyl” refers to a heterocyclyl radical as defined above containing at least one heteroatom and where the point of attachment of the heterocyclyl radical to the rest of the molecule is through a carbon atom in the heterocyclyl radical. A C-heterocyclyl radical is optionally substituted as described above for heterocyclyl radicals. Examples of such C-heterocyclyl radicals include, but are not limited to, 2-morpholinyl, 2- or 3- or 4-piperidinyl, 2-piperazinyl, 2- or 3-pyrrolidinyl, and the like.

“Heterocyclylalkyl” refers to a radical of the formula —R^{c}-heterocyclyl where R^{c} is an alkylene chain as defined above. If the heterocyclyl is a nitrogen-containing heterocyclyl, the heterocyclyl is optionally attached to the alkyl radical at the nitrogen atom. The alkylene chain of the heterocyclylalkyl radical is optionally substituted as defined above for an alkylene chain. The heterocyclyl part of the heterocyclylalkyl radical is optionally substituted as defined above for a heterocyclyl group.

“Heterocyclylalkoxy” refers to a radical bonded through an oxygen atom of the formula —O—R^{c}-heterocyclyl where R^{c} is an alkylene chain as defined above. If the heterocyclyl is a nitrogen-containing heterocyclyl, the heterocyclyl is optionally attached to the alkyl radical at the nitrogen atom. The alkylene chain of the heterocyclylalkoxy radical is optionally substituted as defined above for an alkylene chain. The heterocyclyl part of the heterocyclylalkoxy radical is optionally substituted as defined above for a heterocyclyl group.

“Heteroaryl” refers to a radical derived from a 3- to 18-membered aromatic ring radical that comprises two to seventeen carbon atoms and from one to six heteroatoms selected from nitrogen, oxygen, and sulfur. As used herein, the heteroaryl radical is a monocyclic, bicyclic, tricyclic, or tetracyclic ring system, wherein at least one of the rings in the ring system is fully unsaturated, i.e., it contains a cyclic, delocalized (4n+2) π-electron system in accordance with the Hückel theory. Heteroaryl includes fused or bridged ring systems. The heteroatom(s) in the heteroaryl radical is optionally oxidized. One or more nitrogen atoms, if present, are optionally quaternized. The heteroaryl is attached to the rest of the molecule through any atom of the ring(s). Examples of heteroaryls include, but are not limited to, azepinyl, acridinyl, benzimidazolyl, benzindolyl, 1,3-benzodioxolyl, benzofuranyl, benzooxazolyl, benzo[d]thiazolyl, benzothiadiazolyl, benzo[h][1,4]dioxepinyl, benzo[b][1,4]oxazinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzothieno[3,2-d]pyrimidinyl, benzotriazolyl, benzo[4,6]imidazo[1,2-a]pyridinyl, carbazolyl, cinnolinyl, cyclopenta[d]pyrimidinyl, 6,7-dihydro-5H-cyclopenta[4,5]thieno[2,3-d]pyrimidinyl, 5,6-dihydrobenzo[h]quinazolinyl, 5,6-dihydrobenzo[h]cinnolinyl, 6,7-dihydro-5H-benzo[6,7]cyclohepta[1,2-c]pyridazinyl, dibenzofuranyl, dibenzothiophenyl, furanyl, furanonyl, furo[3,2-c]pyridinyl, 5,6,7,8,9,10-hexahydrocycloocta[d]pyrimidinyl, 5,6,7,8,9,10-hexahydrocycloocta[d]pyridazinyl, 5,6,7,8,9,10-hexahydrocycloocta[d]pyridinyl, isothiazolyl, imidazolyl, indazolyl, indolyl, indazolyl, isoindolyl, indolinyl, isoindolinyl, isoquinolyl, indolizinyl, isoxazolyl, 5,8-methano-5,6,7,8-tetrahydroquinazolinyl, naphthyridinyl, 1,6-naphthyridinonyl, oxadiazolyl, 2-oxoazepinyl, oxazolyl, oxiranyl, 5,6,6a,7,8,9,10,10a-octahydrobenzo[h]quinazolinyl, 1-phenyl-1H-pyrrolyl, phenazinyl, phenothiazinyl, phenoxazinyl, phthalazinyl, pteridinyl, purinyl, pyrrolyl, pyrazolyl, pyrazolo[3,4-d]pyrimidinyl, pyridinyl, pyrido[3,2-d]pyrimidinyl, pyrido[3,4-d]pyrimidinyl, pyrazinyl, pyrimidinyl, pyridazinyl, pyrrolyl, quinazolinyl, quinoxalinyl, quinolinyl, isoquinolinyl, tetrahydroquinolinyl, 5,6,7,8-tetrahydroquinazolinyl, 5,6,7,8-tetrahydrobenzo[4,5]thieno[2,3-d]pyrimidinyl, 6,7,8,9-tetrahydro-5H-cyclohepta[4,5]thieno[2,3-d]pyrimidinyl, 5,6,7,8-tetrahydropyrido[4,5-c]pyridazinyl, thiazolyl, thiadiazolyl, triazolyl, tetrazolyl, triazinyl, thieno[2,3-d]pyrimidinyl, thieno[3,2-d]pyrimidinyl, thieno[2,3-c]pridinyl, and thiophenyl (i.e. thienyl). Unless stated otherwise specifically in the specification, the term “heteroaryl” is meant to include heteroaryl radicals as defined above which are optionally substituted by one or more substituents selected from optionally substituted alkyl, optionally substituted cycloalkylalkyl, optionally substituted heterocyclylalkyl, optionally substituted alkenyl, optionally substituted alkynyl, halo, optionally substituted fluoroalkyl, optionally substituted haloalkenyl, optionally substituted haloalkynyl, oxo, thioxo, cyano, nitro, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(OR^{a} (where t is 1 or 2), —R^{b}—S(O)ₜR^{a} (where t is 1 or 2) and —R^{b}—S(O)N(R^{a})₂ (where t is 1 or 2), where each R^{a} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl), each R^{b} is independently a direct bond or a straight or branched alkylene or alkenylene chain, and R^{c} is a straight or branched alkylene or alkenylene chain, and where each of the R^{a}, R^{b}, or R^{c} substituents is unsubstituted unless otherwise indicated.

“N-heteroaryl” refers to a heteroaryl radical as defined above containing at least one nitrogen and where the point of attachment of the heteroaryl radical to the rest of the molecule is through a nitrogen atom in the heteroaryl radical. An N-heteroaryl radical is optionally substituted as described above for heteroaryl radicals.

“C-heteroaryl” refers to a heteroaryl radical as defined above and where the point of attachment of the heteroaryl radical to the rest of the molecule is through a carbon atom in the heteroaryl radical. A C-heteroaryl radical is optionally substituted as described above for heteroaryl radicals.

“Heteroarylalkyl” refers to a radical of the formula —R^{c}-heteroaryl, where R^{c} is an alkylene chain as defined above. If the heteroaryl is a nitrogen-containing heteroaryl, the heteroaryl is optionally attached to the alkyl radical at the nitrogen atom. The alkylene chain of the heteroarylalkyl radical is optionally substituted as defined above for an alkylene chain. The heteroaryl part of the heteroarylalkyl radical is optionally substituted as defined above for a heteroaryl group.

“Heteroarylalkoxy” refers to a radical bonded through an oxygen atom of the formula —O—R^{c}-heteroaryl, where R^{c} is an alkylene chain as defined above. If the heteroaryl is a nitrogen-containing heteroaryl, the heteroaryl is optionally attached to the alkyl radical at the nitrogen atom. The alkylene chain of the heteroarylalkoxy radical is optionally substituted as defined above for an alkylene chain. The heteroaryl part of the heteroarylalkoxy radical is optionally substituted as defined above for a heteroaryl group.

The compounds disclosed herein, in some embodiments, contain one or more asymmetric centers and thus give rise to enantiomers, diastereomers, and other stereoisomeric forms that are defined, in terms of absolute stereochemistry, as (R)- or (S)—. Unless stated otherwise, it is intended that all stereoisomeric forms of the compounds disclosed herein are contemplated by this disclosure. When the compounds described herein contain alkene double bonds, and unless specified otherwise, it is intended that this disclosure includes both E and Z geometric isomers (e.g., cis or trans.) Likewise, all possible isomers, as well as their racemic and optically pure forms, and all tautomeric forms are also intended to be included. The term “geometric isomer” refers to E or Z geometric isomers (e.g., cis or trans) of an alkene double bond. The term “positional isomer” refers to structural isomers around a central ring, such as ortho-, meta-, and para-isomers around a benzene ring.

As used herein, “carboxylic acid bioisostere” refers to a functional group or moiety that exhibits similar physical, biological and/or chemical properties as a carboxylic acid moiety. Examples of carboxylic acid bioisosteres include, but are not limited to,

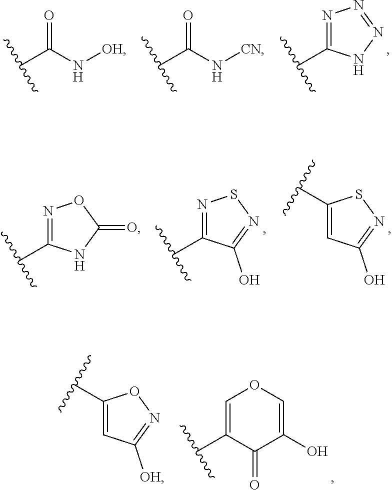

and the like.

A “tautomer” refers to a molecule wherein a proton shift from one atom of a molecule to another atom of the same molecule is possible. The compounds presented herein, in certain embodiments, exist as tautomers. In circumstances where tautomerization is possible, a chemical equilibrium of the tautomers will exist. The exact ratio of the tautomers depends on several factors, including physical state, temperature, solvent, and pH. Some examples of tautomeric equilibrium include:

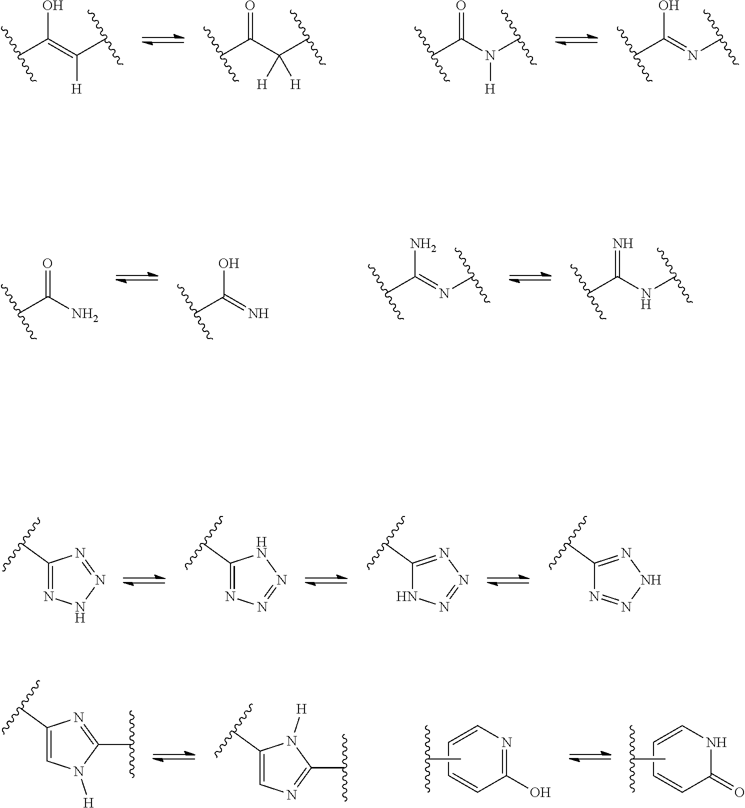

The compounds disclosed herein, in some embodiments, are used in different enriched isotopic forms, e.g., enriched in the content of ²H, ³H, ¹¹C, ¹³C and/or ¹⁴C. In one particular embodiment, the compound is deuterated in at least one position. Such deuterated forms can be made by the procedure described in U.S. Pat. Nos. 5,846,514 and 6,334,997. As described in U.S. Pat. Nos. 5,846,514 and 6,334,997, deuteration can improve the metabolic stability and or efficacy, thus increasing the duration of action of drugs.

Unless otherwise stated, structures depicted herein are intended to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of the present disclosure.

The compounds of the present disclosure optionally contain unnatural proportions of atomic isotopes at one or more atoms that constitute such compounds. For example, the compounds may be labeled with isotopes, such as for example, deuterium (²H), tritium (3H), iodine-125 (¹²⁵I) or carbon-14 (¹⁴C). Isotopic substitution with ²H, ¹¹C, ¹³C, ¹⁴C, ¹⁵C, ¹²N, ¹³N, ¹⁵N, ¹⁶N, ¹⁶O, ¹⁷O, ¹⁴F, ¹⁵F, ¹⁶F, ¹⁷F, ¹⁸F, ³³S, ³⁴S, ³⁵S, ³⁶S, ³⁵Cl, ³⁷Cl, ⁷⁹Br, ⁸¹Br, ¹²⁵I are all contemplated. In some embodiments, isotopic substitution with ¹⁸F is contemplated. All isotopic variations of the compounds of the present invention, whether radioactive or not, are encompassed within the scope of the present invention.

In certain embodiments, the compounds disclosed herein have some or all of the ¹H atoms replaced with ²H atoms. The methods of synthesis for deuterium-containing compounds are known in the art and include, by way of non-limiting example only, the following synthetic methods.

Deuterium substituted compounds are synthesized using various methods such as described in: Dean, Dennis C.; Editor. Recent Advances in the Synthesis and Applications of Radiolabeled Compounds for Drug Discovery and Development. [Curr., Pharm. Des., 2000; 6 (10)] 2000, 110 pp; George W.; Varma, Rajender S. The Synthesis of Radiolabeled Compounds via Organometallic Intermediates, Tetrahedron, 1989, 45(21), 6601-21; and Evans, E. Anthony. Synthesis of radiolabeled compounds, J. Radioanal. Chem., 1981, 64 (1-2), 9-32.

Deuterated starting materials are readily available and are subjected to the synthetic methods described herein to provide for the synthesis of deuterium-containing compounds. Large numbers of deuterium-containing reagents and building blocks are available commercially from chemical vendors, such as Aldrich Chemical Co.

Deuterium-transfer reagents suitable for use in nucleophilic substitution reactions, such as iodomethane-d3 (CD₃I), are readily available and may be employed to transfer a deuterium-substituted carbon atom under nucleophilic substitution reaction conditions to the reaction substrate. The use of CD₃I is illustrated, by way of example only, in the reaction schemes below.

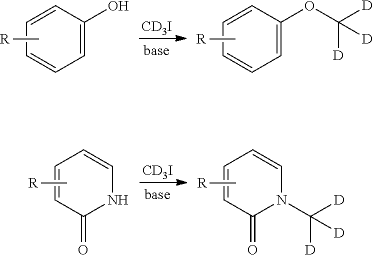

Deuterium-transfer reagents, such as lithium aluminum deuteride (LiAlD₄), are employed to transfer deuterium under reducing conditions to the reaction substrate. The use of LiAlD₄ is illustrate by way of example only, in the reaction schemes below.

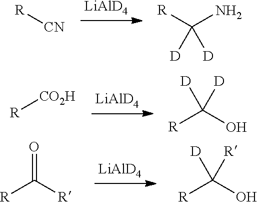

Deuterium gas and palladium catalyst are employed to reduce unsaturated carbon-carbon linkages and to perform a reductive substitution of aryl carbon-halogen bonds as illustrated, by way of example only, in the reaction schemes below.

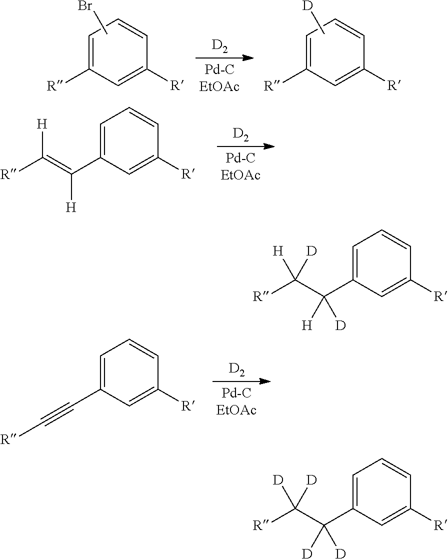

In one embodiment, the compounds disclosed herein contain one deuterium atom. In another embodiment, the compounds disclosed herein contain two deuterium atoms. In another embodiment, the compounds disclosed herein contain three deuterium atoms. In another embodiment, the compounds disclosed herein contain four deuterium atoms. In another embodiment, the compounds disclosed herein contain five deuterium atoms. In another embodiment, the compounds disclosed herein contain six deuterium atoms. In another embodiment, the compounds disclosed herein contain more than six deuterium atoms. In another embodiment, the compound disclosed herein is fully substituted with deuterium atoms and contains no non-exchangeable ¹H hydrogen atoms. In one embodiment, the level of deuterium incorporation is determined by synthetic methods in which a deuterated synthetic building block is used as a starting material.

“Pharmaceutically acceptable salt” includes both acid and base addition salts. A pharmaceutically acceptable salt of any one of the AKT1 inhibitory compounds described herein is intended to encompass any and all pharmaceutically suitable salt forms. Preferred pharmaceutically acceptable salts of the compounds described herein are pharmaceutically acceptable acid addition salts and pharmaceutically acceptable base addition salts.

“Pharmaceutically acceptable acid addition salt” refers to those salts which retain the biological effectiveness and properties of the free bases, which are not biologically or otherwise undesirable, and which are formed with inorganic acids such as hydrochloric acid, hydrobromic acid, sulfuric acid, nitric acid, phosphoric acid, hydroiodic acid, hydrofluoric acid, phosphorous acid, and the like. Also included are salts that are formed with organic acids such as aliphatic mono- and dicarboxylic acids, phenyl-substituted alkanoic acids, hydroxy alkanoic acids, alkanedioic acids, aromatic acids, aliphatic and aromatic sulfonic acids, etc. and include, for example, acetic acid, trifluoroacetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Exemplary salts thus include sulfates, pyrosulfates, bisulfates, sulfites, bisulfites, nitrates, phosphates, monohydrogenphosphates, dihydrogenphosphates, metaphosphates, pyrophosphates, chlorides, bromides, iodides, acetates, trifluoroacetates, propionates, caprylates, isobutyrates, oxalates, malonates, succinate suberates, sebacates, fumarates, maleates, mandelates, benzoates, chlorobenzoates, methylbenzoates, dinitrobenzoates, phthalates, benzenesulfonates, toluenesulfonates, phenylacetates, citrates, lactates, malates, tartrates, methanesulfonates, and the like. Also contemplated are salts of amino acids, such as arginates, gluconates, and galacturonates (see, for example, Berge S. M. et al., “Pharmaceutical Salts,” Journal of Pharmaceutical Science, 66:1-19 (1997)). Acid addition salts of basic compounds are, in some embodiments, prepared by contacting the free base forms with a sufficient amount of the desired acid to produce the salt according to methods and techniques with which a skilled artisan is familiar.

“Pharmaceutically acceptable base addition salt” refers to those salts that retain the biological effectiveness and properties of the free acids, which are not biologically or otherwise undesirable. These salts are prepared from addition of an inorganic base or an organic base to the free acid. Pharmaceutically acceptable base addition salts are, in some embodiments, formed with metals or amines, such as alkali and alkaline earth metals or organic amines. Salts derived from inorganic bases include, but are not limited to, sodium, potassium, lithium, ammonium, calcium, magnesium, iron, zinc, copper, manganese, aluminum salts and the like. Salts derived from organic bases include, but are not limited to, salts of primary, secondary, and tertiary amines, substituted amines including naturally occurring substituted amines, cyclic amines and basic ion exchange resins, for example, isopropylamine, trimethylamine, diethylamine, triethylamine, tripropylamine, ethanolamine, diethanolamine, 2-dimethylaminoethanol, 2-diethylaminoethanol, dicyclohexylamine, lysine, arginine, histidine, caffeine, procaine, N,N-dibenzylethylenediamine, chloroprocaine, hydrabamine, choline, betaine, ethylenediamine, ethylenedianiline, N-methylglucamine, glucosamine, methylglucamine, theobromine, purines, piperazine, piperidine, N-ethylpiperidine, polyamine resins and the like. See Berge et al., supra.

“Pharmaceutically acceptable solvate” refers to a composition of matter that is the solvent addition form. In some embodiments, solvates contain either stoichiometric or non-stoichiometric amounts of a solvent, and are formed during the process of making with pharmaceutically acceptable solvents such as water, ethanol, and the like. Hydrates are formed when the solvent is water, or alcoholates are formed when the solvent is alcohol. Solvates of compounds described herein are conveniently prepared or formed during the processes described herein. The compounds provided herein exist in either unsolvated or solvated forms.

The term “subject” or “patient” encompasses mammals. Examples of mammals include, but are not limited to, any member of the mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. In one aspect, the mammal is a human.

As used herein, “treatment” or “treating,” or “palliating” or “ameliorating” are used interchangeably. These terms refer to an approach for obtaining beneficial or desired results including but not limited to therapeutic benefit and/or a prophylactic benefit. By “therapeutic benefit” is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient is still afflicted with the underlying disorder. For prophylactic benefit, the compositions are, in some embodiments, administered to a patient at risk of developing a particular disease, or to a patient reporting one or more of the physiological symptoms of a disease, even though a diagnosis of this disease has not been made.

### AKT1 Protein and Function

AKT, also known as protein kinase B (PKB), is a serine/threonine protein kinase with three isoforms, AKT1, AKT2, and AKT3. While the isoforms are encoded by different genes, they are highly homologous at the protein level and share a conserved domain structure comprising an N-terminal pleckstrin homology (PH) domain, a kinase domain, and a C-terminal regulatory domain comprising a hydrophobic moiety, which includes the regulatory serine residue (Nitulescu, G. M. et al., Int J Oncol., 2018; 53(6): 2319-2331).

AKT proteins play a crucial role in major cellular functions including cell cycle progression, cell size, regulation of glucose metabolism, transcription, protein synthesis, genome stability, and neovascularization. AKT proteins can block apoptosis by inactivation of pro-apoptotic proteins, and mediate cellular growth factors, promoting cell survival. AKT is a major downstream effector of nuclear factor-kappaB (NfKB), which may link AKT signaling to the nucleus of a cell.

AKT1 is ubiquitously expressed, whereas AKT2 is primarily expressed in insulin-responsive tissues, and AKT3 is primarily expressed in brain and testes. A shared phosphorylation site of AKT in the catalytic domain corresponds to a threonine residue; specifically, Thr308 in AKT1, Thr309 in AKT2, and Thr305 in AKT3. A shared phosphorylation site in the C-terminus of the protein cis a serine residue; specifically, Ser473 in AKT1, Ser474 in AKT2, and Ser472 in AKT3.

AKT is a key downstream mediator of the phosphoinositide-3-kinase (PI3K) signaling pathway. PI3Ks are activated by different compounds. For example, PI3Kα, PI3Kβ, and PI3Kδ, are activated by extracellular ligands binding to a transmembrane glycoprotein with enzymatic activity, receptor tyrosine kinases (RTKs). In contrast, PI3Kγ is activated by G-protein-compound receptors (GPCRs) and by RAS family of GTPases.

The AKT cascade can be activated by RTKs and G-protein-compound receptors (GPCRs), along with other signals including integrins, B cell receptors, T cell receptors, and cytokine receptors.

### AKT1 Mechanism

AKT is activated by a second phosphorylation at the regulatory serine residue, Ser473. Known phosphorylating agents of AKT at Ser473 include, but are not limited to PDK-1, integrin-linked kinase (ILK), members of the PI3K-related kinase (PIKK) family, and mammalian target of rapamycin (mTOR) (Nitulescu, G. M. et al., Int J Oncol., 2018; 53(6): 2319-2331).

mTOR is a key component in the AKT signaling pathway, which is a downstream member of AKT and important regulator for cell metabolism and growth. mTOR is also an activator which can directly phosphorylate AKT's regulatory serine residue, Ser473. mTOR forms a complex with rapamycin-insensitive companion of mTOR (RICTOR) (and other proteins) to form mTOR complex 2 (mTORC2), which can directly phosphorylate AKT Ser473. AKT can affect cell survival and growth because it can influence the tuberous sclerosis complex (TSC) 1/2 along the mTORC signaling pathway and inhibit pro-apoptotic proteins or signals.

AKT is known as a survival kinase and mediates cell survival and proliferation by inhibiting pathways including, but not limited to Bcl2 and MDM2, which promotes apoptosis. Studies have shown that the AKT signaling cascade have frequent malfunctions in various cancers, and may be associated with tumor aggressiveness (Nitulescu, G. M. et al., Int J Oncol., 2018; 53(6): 2319-2331). Malfunctions of AKT typically lead to enhanced proliferation, growth, survival, and resistance to apoptosis (Alwhaibi, A. et al., Pharmacol Res., 2019, 145: 104270). Malfunction and mis-regulation of AKT may lead to cancers such as but not limited to breast cancer, gastric carcinoma, glioblastoma, gliosarcomas, head and neck squamous cell carcinoma, ovarian cancer, pancreatic cancer, and prostate cancer.

Additionally, AKT1 has been found to be involved in invasion and migration of cancerous cells (Alwhaibi, A. et al., Pharmacol Res., 2019, 145: 104270). Researchers found that silencing the AKT1 isoform can abrogate specific types of cancer cell migration. However, there have been other studies which have demonstrated that activated AKT1 resulted in less metastatic propensity for lung metastatic lesion cells and breast cancer cells. AKT1 has also been identified as a key protein involved in angiogenesis, lung cancer, and tumorigenesis.

Furthermore, overexpression of AKT has been correlated to resistance to chemotherapeutic agents such as cisplatin, methotrexate, and paclitaxel. Thus, there remains a need to find AKT inhibitors given its role in cell survival and cancer proliferation.

Recently, it has been found that the AKT1 gene mutation E17K can affect cell growth, proliferation, survival, and migration of breast cancer cells, colorectal cancer cells, and ovarian cancer cells (Chen, Y. et al., Front Cell Dev Biol., 2020: 8: 573599). These mutations in the PH structural domain increase the binding of AKT1 to Phosphatidylinositol-3,4,5-triphosphate (PIP3) lipid ligand, which accelerates transfer of AKT from the cytoplasm to the cell membrane through formation of hydrogen bonds. Transfer of AKT into the cell membrane allows it to be further phosphorylated. Once fully activated, AKT can return to the cytoplasm, or go to the nucleus or other intracellular sites, and phosphorylate other substrate proteins to regulate cell function.

The E17K mutation enhances migration of breast cancer cells, and also enhances resistance to chemotherapeutic drugs. However, the E17K mutation can also selectively destroy chemo-resistant tumor-promoting AKT1 quiescent cancer cells, suggesting that the AKT1(E17K) mutation is crucial in the oncogenic/anti-tumor mechanism.

A major pathway that activates PI3K-AKT signaling pathway is somatic cell mutations, with the E17K mutation being the highest frequency of AKT1 mutations. It is nearly exclusively present in AKT1. The AKT1(E17K) is a recurrent somatic cell mutation predominantly in breast cancer, ovarian cancer, meningioma, and Proteus syndrome.

AKT1(E17K) mutations mediate the PI3K-AKT signaling cascade by expanding PIP lipid specificity, which causes conformational changes. This also enhances subcellular localization to accelerate localization of the PH structural domain to the plasma membrane. The E17K mutation increases PIP3 binding specificity by 7-fold and phosphatidylinositol-(4,5)-bisphosphate (PIP2) by 100-fold.

The AKT1(E17K) mutation also causes rapid conformational changes in the AKT1 PH structural domain. The conformational changes to this domain result in a 4.5-fold increase in its membrane localization, which can result in excessive phosphorylation. The AKT1(E17K) mutation can also result in enhanced subcellular localization by increasing the transient expression.

Given the conformational and signaling effects of the AKT1(E17K) mutation, this target may be useful for targeted treatment of cancers.

### Prior Art AKT1 Inhibitors

Most AKT inhibitors targeting the ATP binding site are non-selective against the three isoforms, as well as having poor to no selectivity against other structurally similar kinases. Thus, there remains a need to develop new and novel AKT inhibitors. These ATP targeting inhibitors are classified as aminofurazans, azepane derivatives, isoquinoline-5-sulfonamides, phenylpyrazole derivatives, thiophene carboxamide derivatives, and thiazole carboxamide derivatives.

There are also ATP non-competitive AKT inhibitors which are allosteric modulators which has greater specificity than the ATP targeting inhibitors. Many of these allosteric modulator inhibitors are classified as purine derivatives, thiourea derivatives, alkylphospholipids, sulfonamides, 2,3-diphenylquinoxaline analogs, and indole-3-carbinol derivatives.

### Novel AKT1 Inhibitory Compounds

In one aspect, provided herein is a AKT1 inhibitory compound.

One embodiment provides a compound having the structure of Formula (I), or a pharmaceutically acceptable salt or solvate thereof:

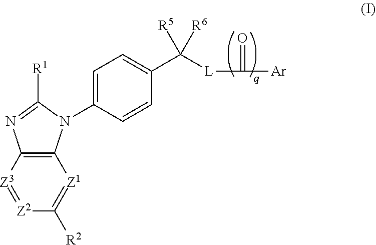

wherein:
   Z¹ is N, C—H, or C—R³;
   Z² is N, C—H, or C—R⁴;
   Z³ is N, C—OH, or C—R⁹;
   Ar is selected from:

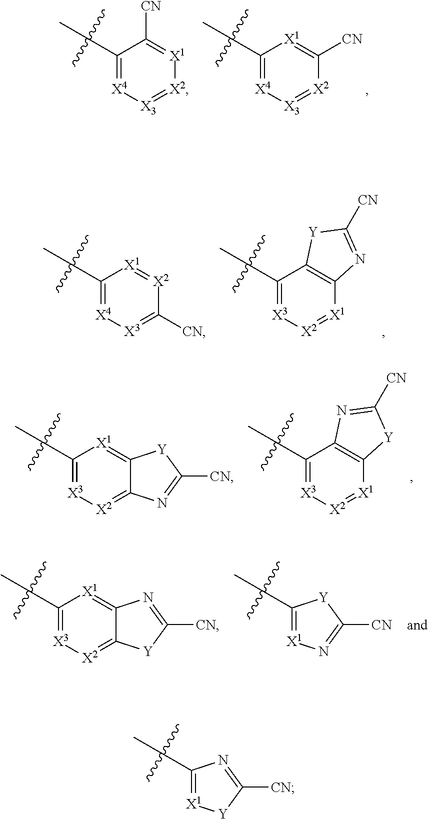

X¹ is N or C—R⁷;
X² is N or C—R⁷;
X³ is N or C—R⁷;
X⁴ is N or C—R⁷;
Y is O, S, or N—R⁹;
R¹ is selected from hydrogen, optionally substituted C₁-C₆ alkyl, optionally substituted aryl, or optionally substituted heteroaryl;
R² is selected from hydrogen, halogen, —OH, —CN, —N(R⁹)₂, —OR⁹, —SR⁹, —SO₂R⁹, —CO₂R⁹, —CON(R⁹)₂, —SR⁹, —S(O)R⁹, —S(O)₂R⁹, optionally substituted C1-C6 alkyl, optionally substituted C1-C6 alkoxy, optionally substituted C2-C6 alkenyl, optionally substituted C2-C6 alkynyl, optionally substituted C3-C7 carbocyclyl, optionally substituted 4-membered to 6-membered heterocyclyl, optionally substituted aryl, aryl substituted with an optionally substituted 4-membered to 6-membered heterocyclyl, optionally substituted heteroaryl or heteroaryl substituted with an optionally substituted 3-membered to 6-membered carbocyclyl;
R³ is selected from optionally substituted C1-C6 alkyl, or optionally substituted aryl;
R⁴ is selected from halogen, —CN, optionally substituted C1-C6 alkyl, or optionally substituted aryl;
R⁵ and R⁶ are each independently hydrogen, deuterium, halogen, —OH, or optionally substituted C1-C6 alkyl; or R⁵ and R⁶ together form an oxo; or R⁵ and R⁶ join together to form a carbocycle or heterocycle;
each R⁷ is independently selected from hydrogen, deuterium, halogen, —OH, —SH, optionally substituted C1-C6 alkoxy, —S— (optionally substituted C1-C6 alkyl), —CN, optionally substituted C1-C6 alkyl, and optionally substituted aryl;
L is selected from —N(R⁸)—, or a divalent radical selected from:

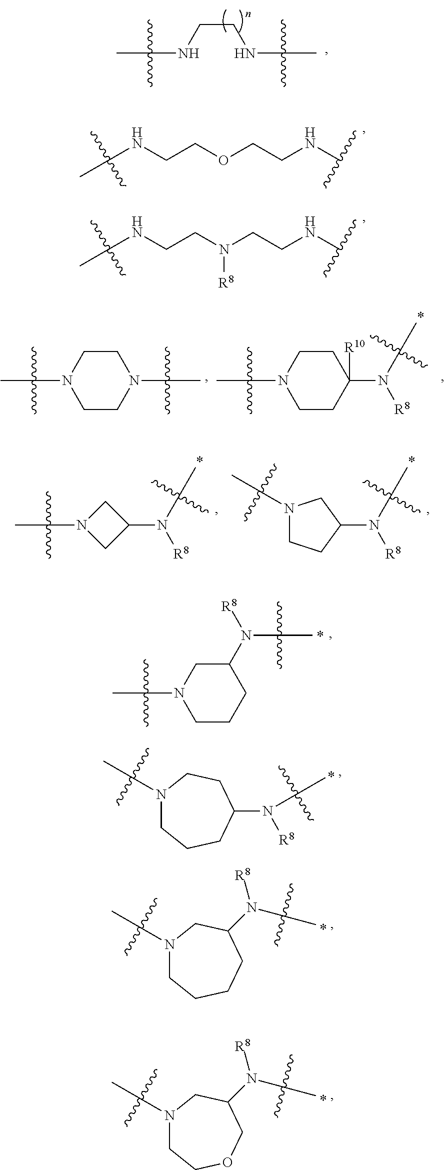

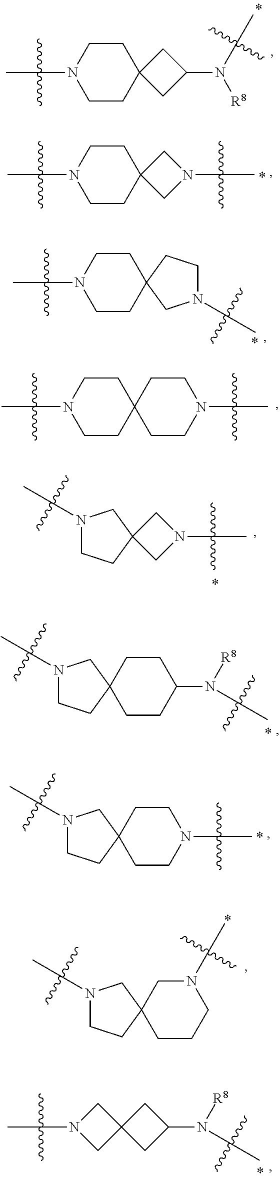

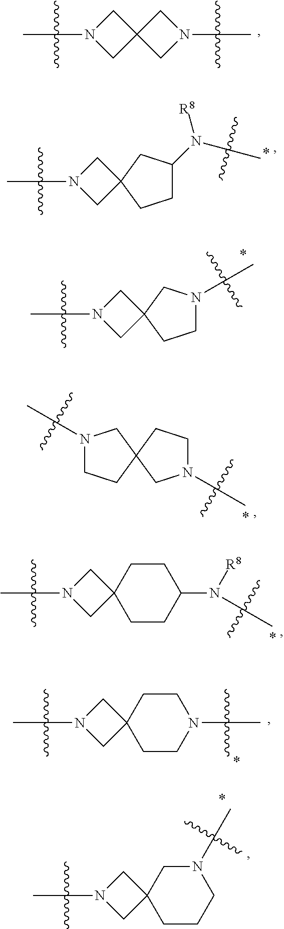

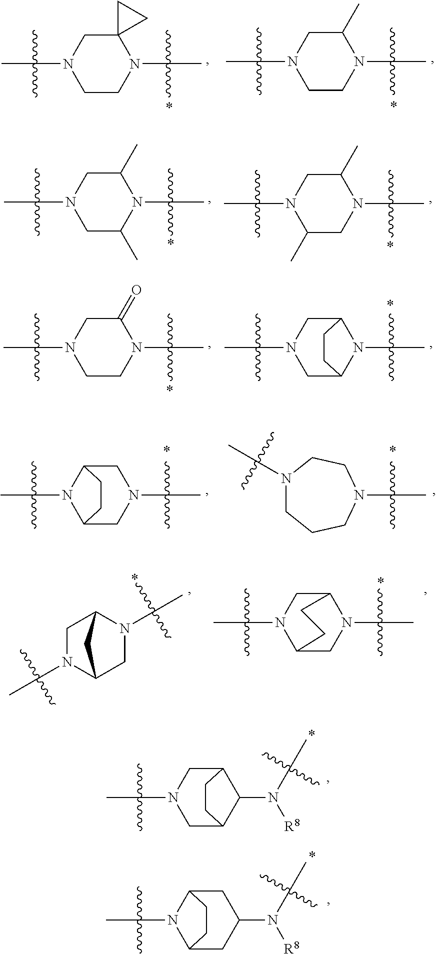

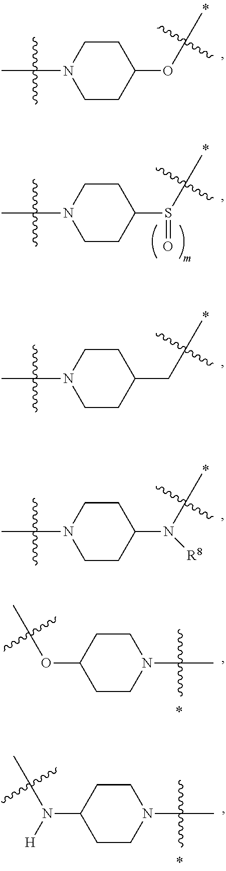

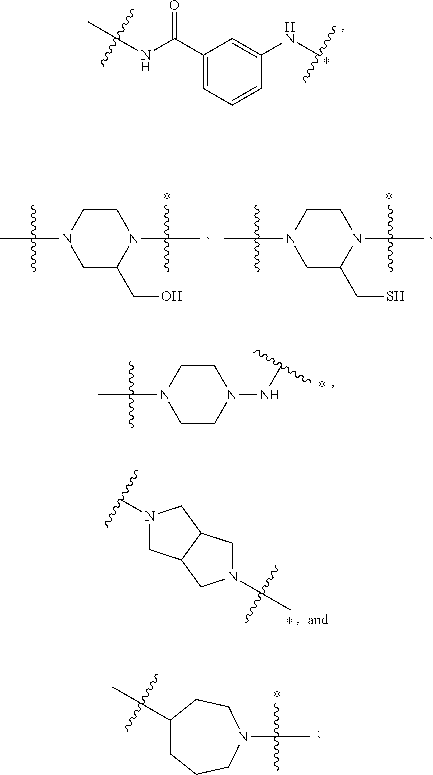 [SMILES: *NC(=O)c1cccc(N*)c1 ; *N1CCN(*)C(CO)C1 ; *N1CCN(*)C(CS)C1 ; *NN1CCN(*)CC1 ; *N1CC2CN(*)CC2C1 ; *C1CCCN(*)CC1]

wherein the asterisk (*) indicates the bond to the —CO—Ar group;
   R⁸ is hydrogen, optionally substituted C1-C6 alkyl, optionally substituted C3-C7 cycloalkyl, or optionally substituted heterocyclyl;
   each R⁹ is hydrogen, or optionally substituted C1-C6 alkyl;
   R¹⁰ is optionally substituted C1-C6 alkyl;
m is 0, 1, or 2;
n is 1, 2, or 3; and
q is 0 or 1.

One embodiment provides a compound having the structure of Formula (Ia), or a pharmaceutically acceptable salt or solvate thereof:

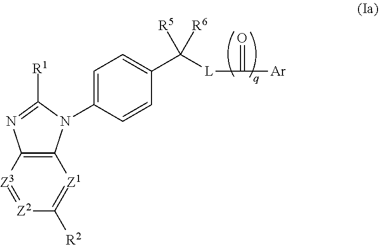

wherein:
   Z¹ is N, C—H, or C—R³;
   Z² is N, C—H, or C—R⁴;
   Z³ is N, C—H;
   Ar is selected from:

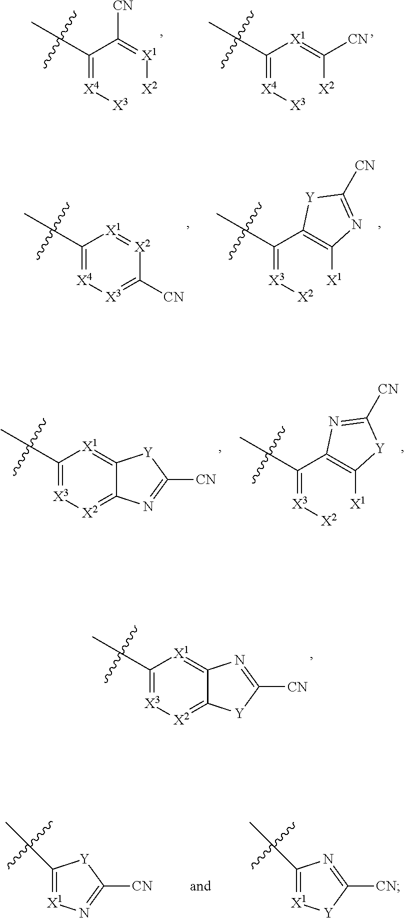

X¹ is N or C—R⁷;
X² is N or C—R⁷;
X⁴ is N or C—R⁷;
Y is O, S, or N—R⁹;
R¹ is selected from hydrogen, optionally substituted C1-C6 alkyl, optionally substituted aryl, or optionally substituted heteroaryl;
R² is selected from hydrogen, halogen, —OH, —CN, —N(R⁹)₂, —OR⁹, —SR⁹, —SO₂R⁹, —CO₂R⁹, —CON(R⁹)₂, optionally substituted C1-C6 alkyl, optionally substituted C3-C7 carbocyclyl, optionally substituted 4-membered to 6-membered heterocyclyl optionally substituted aryl, or optionally substituted heteroaryl;
R³ is selected from optionally substituted C1-C6 alkyl, or optionally substituted aryl;
R⁴ is selected from halogen, —CN, optionally substituted C1-C6 alkyl, or optionally substituted aryl;
R⁵ and R⁶ are each independently hydrogen, deuterium, halogen, —OH, or optionally substituted C1-C6 alkyl; or R⁵ and R⁶ together form an oxo; or R⁵ and R⁶ join together to form a carbocycle or heterocycle;
each R⁷ is independently selected from hydrogen, deuterium, halogen, —OH, —SH, optionally substituted C1-C6 alkoxy, —S— (optionally substituted C1-C6 alkyl), —CN, optionally substituted C1-C6 alkyl, and optionally substituted aryl; L is selected from —N(R⁸)—, or a divalent radical selected from:

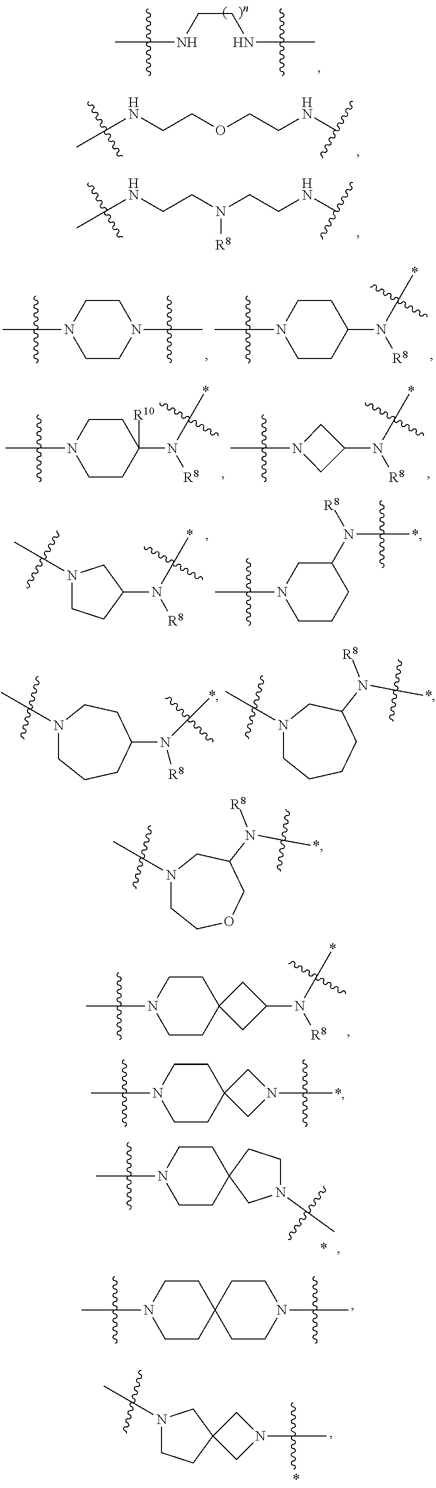

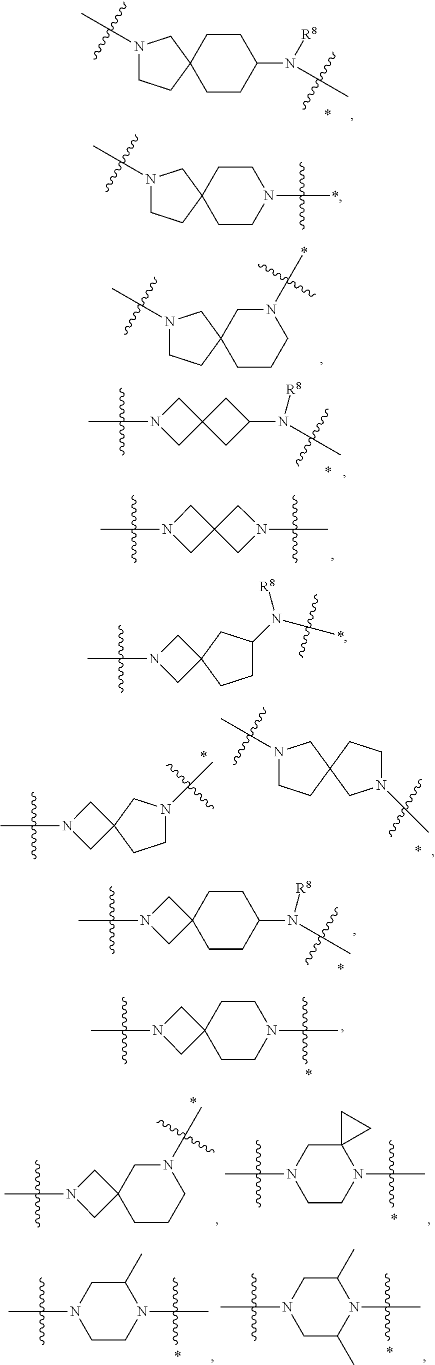

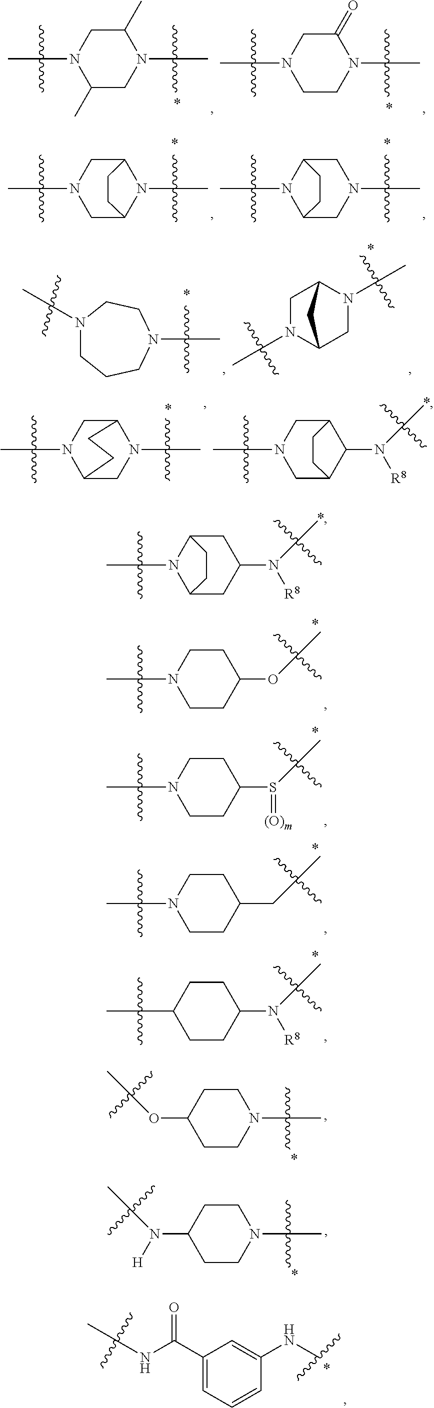

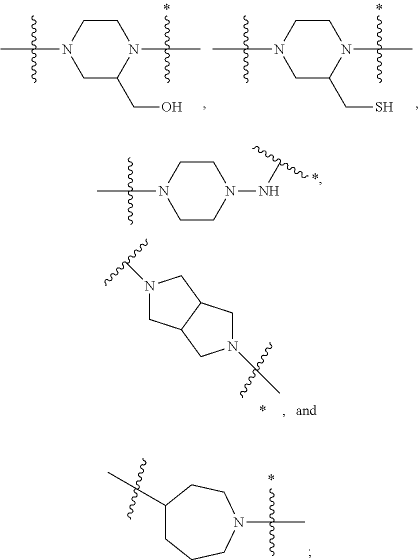 [SMILES: *N1CCN(*)C(CO)C1 ; *N1CCN(*)C(CS)C1 ; *NN1CCN(*)CC1 ; *N1CC2CN(*)CC2C1 ; *C1CCCN(*)CC1]

wherein the asterisk (*) indicates the bond to the —CO—Ar group;
   R⁸ is hydrogen, optionally substituted C1-C6 alkyl, optionally substituted C3-C7 cycloalkyl, or optionally substituted heterocyclyl;
   each R⁹ is hydrogen, or optionally substituted C1-C6 alkyl;
   R¹⁰ is optionally substituted C1-C6 alkyl;
   m is 0, 1, or 2;
   n is 1, 2, or 3; and
   q is 0 or 1.

One embodiment provides a compound having the structure of Formula (Ib), or a pharmaceutically acceptable salt or solvate thereof:

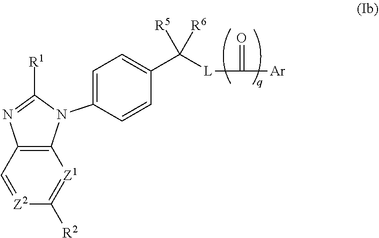

wherein:
   Z¹ is N, C—H, or C—R³;
   Z² is N, C—H, or C—R;
   Ar is selected from:

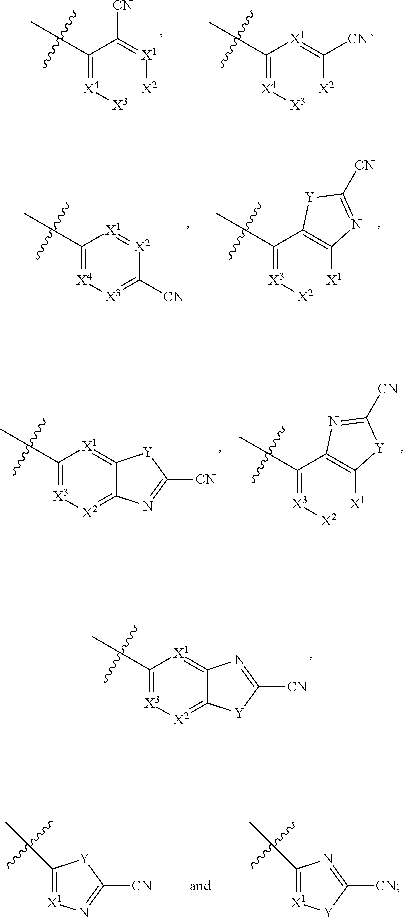

X³ is N or C—R⁷;
X² is N or C—R⁷;
X³ is N or C—R⁷;
X⁴ is N or C—R⁷;
Y is O, S, or N—R⁹;
R¹ is selected from hydrogen, optionally substituted C1-C6 alkyl, optionally substituted aryl, or optionally substituted heteroaryl;
R² is selected from hydrogen, optionally substituted C1-C6 alkyl, optionally substituted aryl, or optionally substituted heteroaryl;
R³ is selected from optionally substituted C1-C6 alkyl, or optionally substituted aryl;
R⁴ is selected from optionally substituted C1-C6 alkyl, or optionally substituted aryl;
R⁵ and R⁶ are each independently hydrogen, deuterium, halogen, —OH, or optionally substituted C1-C6 alkyl; or R⁵ and R⁶ together form an oxo; or R⁵ and R⁶ join together to form a carbocycle or heterocycle;
each R⁷ is independently selected from hydrogen, deuterium, halogen, —OH, —SH, optionally substituted C1-C6 alkoxy, —S— (optionally substituted C1-C6 alkyl), —CN, optionally substituted C1-C6 alkyl, and optionally substituted aryl;
L is selected from —N(R⁸)—, or a divalent radical selected from:

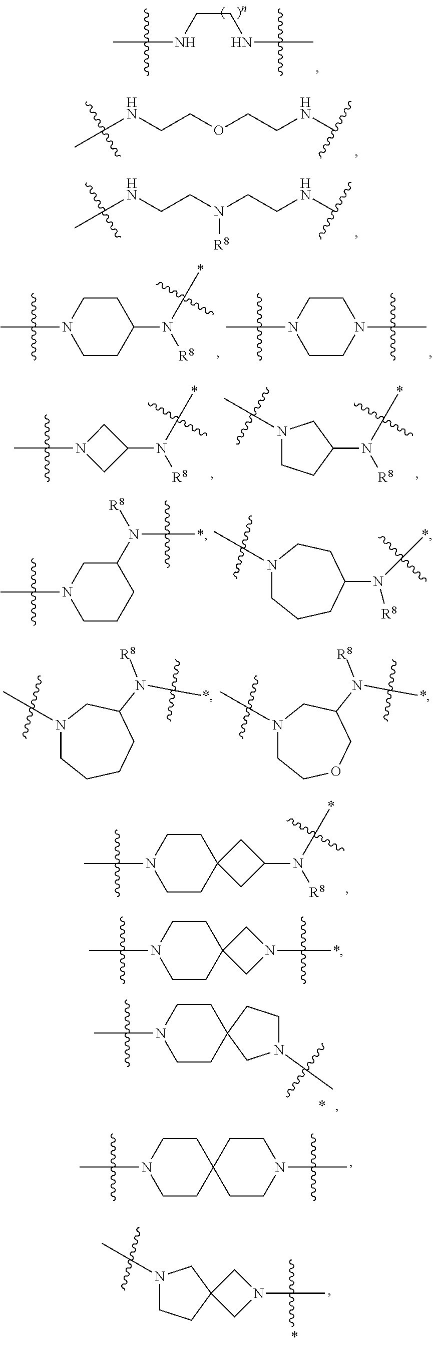

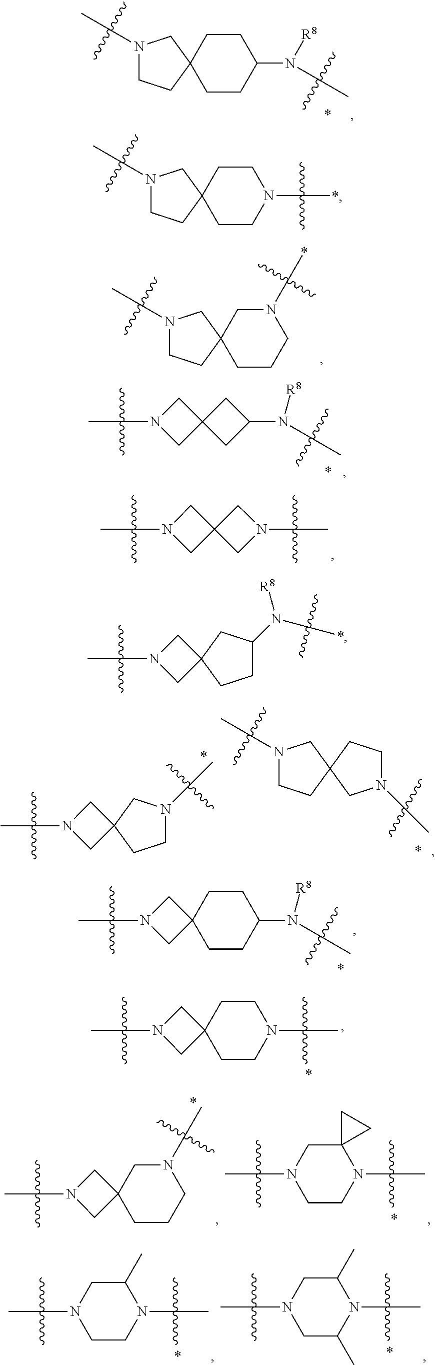

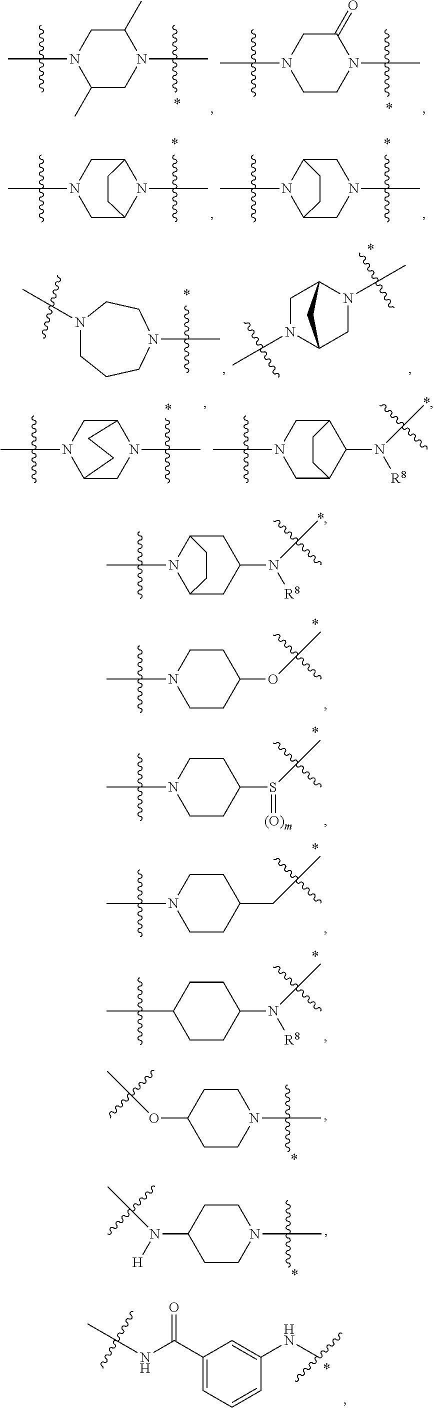

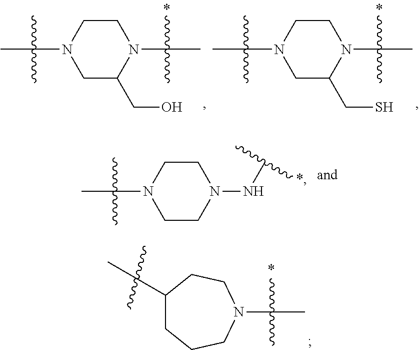 [SMILES: *N1CCN(*)C(CO)C1 ; *N1CCN(*)C(CS)C1 ; *NN1CCN(*)CC1 ; *C1CCCN(*)CC1]

wherein the asterisk (*) indicates the bond to the —CO—Ar group;
   R⁸ is hydrogen, optionally substituted C1-C6 alkyl, optionally substituted C3-C7 cycloalkyl, or optionally substituted heterocyclyl;
   R⁹ is hydrogen, or optionally substituted C1-C6 alkyl;
   m is 0, 1, or 2;
   n is 1, 2, or 3; and
   q is 0 or 1.

Another embodiment provides the compound of Formula (I), (Ia) or (Ib), or pharmaceutically acceptable salt or solvate thereof, wherein Z¹ is N.

Another embodiment provides the compound of Formula (I), (Ia) or (Ib), or pharmaceutically acceptable salt or solvate thereof, wherein Z² is C—H.

Another embodiment provides the compound of Formula (I), (Ia) or (Ib), or pharmaceutically acceptable salt or solvate thereof, wherein Z² is C—R⁴.

Another embodiment provides the compound of Formula (I), (Ia) or (Ib), or pharmaceutically acceptable salt or solvate thereof, wherein Rt is optionally substituted heteroaryl. Another embodiment provides the compound of Formula (I), (Ia) or (Ib), or pharmaceutically acceptable salt or solvate thereof, wherein the optionally substituted heteroaryl is an optionally substituted pyridyl.

Another embodiment provides the compound of Formula (I), (Ia) or (Tb), or pharmaceutically acceptable salt or solvate thereof, wherein R² is optionally substituted aryl. Another embodiment provides the compound of Formula (I), (Ia) or (Ib), or pharmaceutically acceptable salt or solvate thereof, wherein the optionally substituted aryl is an optionally substituted phenyl.

Another embodiment provides the compound of Formula (I), (Ia) or (Ib), or pharmaceutically acceptable salt or solvate thereof, wherein R² is optionally substituted heteroaryl. Another embodiment provides the compound of Formula (I), (Ia) or (Ib), or pharmaceutically acceptable salt or solvate thereof, wherein the optionally substituted heteroaryl is an optionally substituted pyridyl.

Another embodiment provides the compound of Formula (I), (Ia) or (Ib), or pharmaceutically acceptable salt or solvate thereof, wherein R⁵ is hydrogen. Another embodiment provides the compound of Formula (I), (Ia) or (Tb), or pharmaceutically acceptable salt or solvate thereof, wherein R⁶ is hydrogen. Another embodiment provides the compound of Formula (I), (Ia) or (Ib), or pharmaceutically acceptable salt or solvate thereof, wherein R⁵ and R⁶ together form an oxo. Another embodiment provides the compound of Formula (I), (Ia) or (Ib), or pharmaceutically acceptable salt or solvate thereof, wherein R⁶ is optionally substituted C1-C6 alkyl. Another embodiment provides the compound of Formula (I), (Ia) or (Ib), or pharmaceutically acceptable salt or solvate thereof, wherein Rand R⁶ join together to form a carbocycle or heterocycle.

Another embodiment provides the compound of Formula (I), (Ia) or (Ib), or pharmaceutically acceptable salt or solvate thereof, wherein L is —N(R⁸)—.

Another embodiment provides the compound of Formula (I), (Ia) or (Ib), or pharmaceutically acceptable salt or solvate thereof, wherein L is selected from a divalent radical selected from:

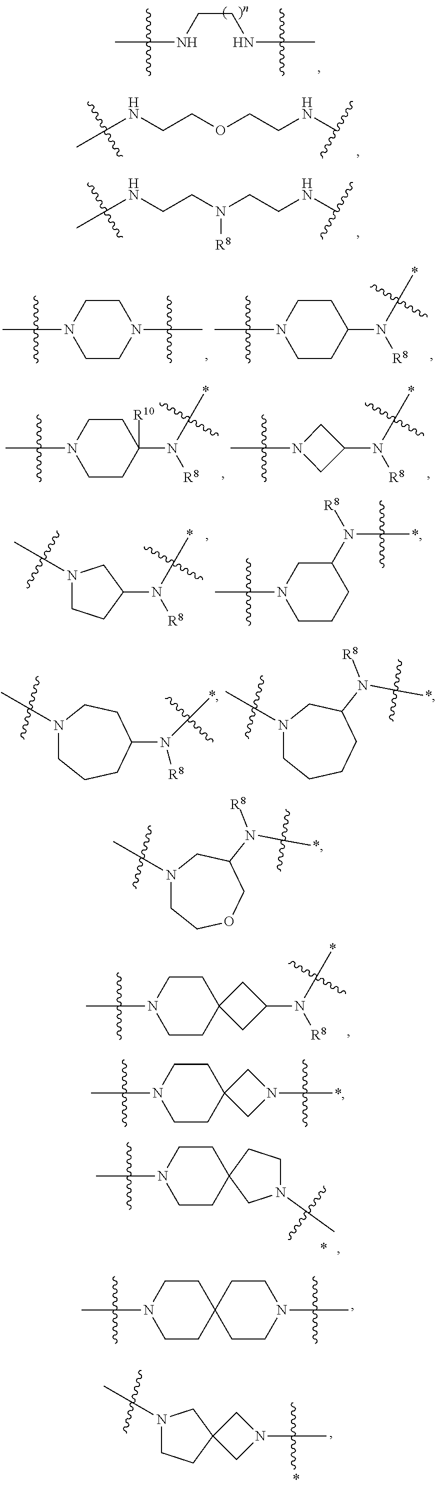

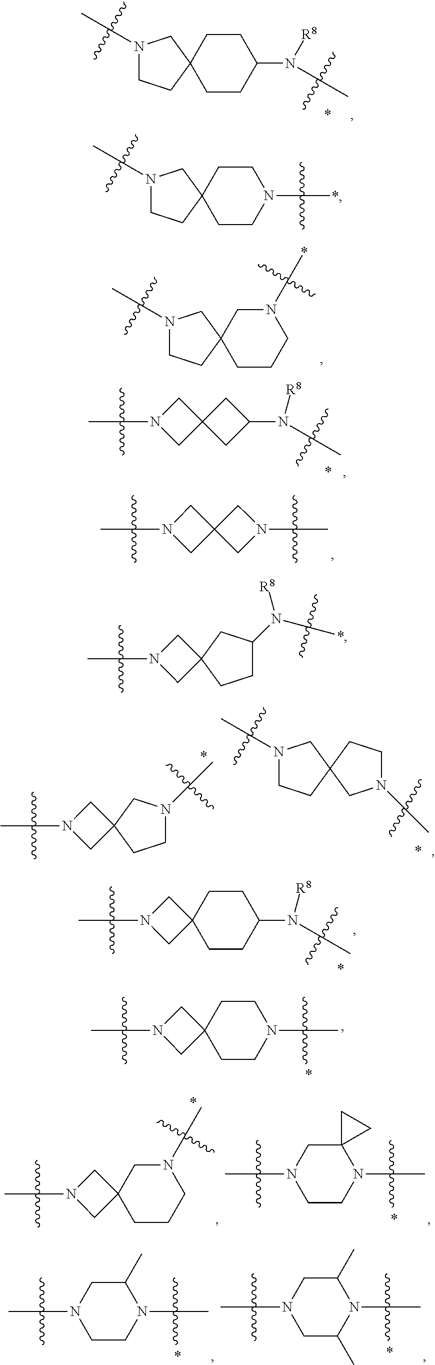

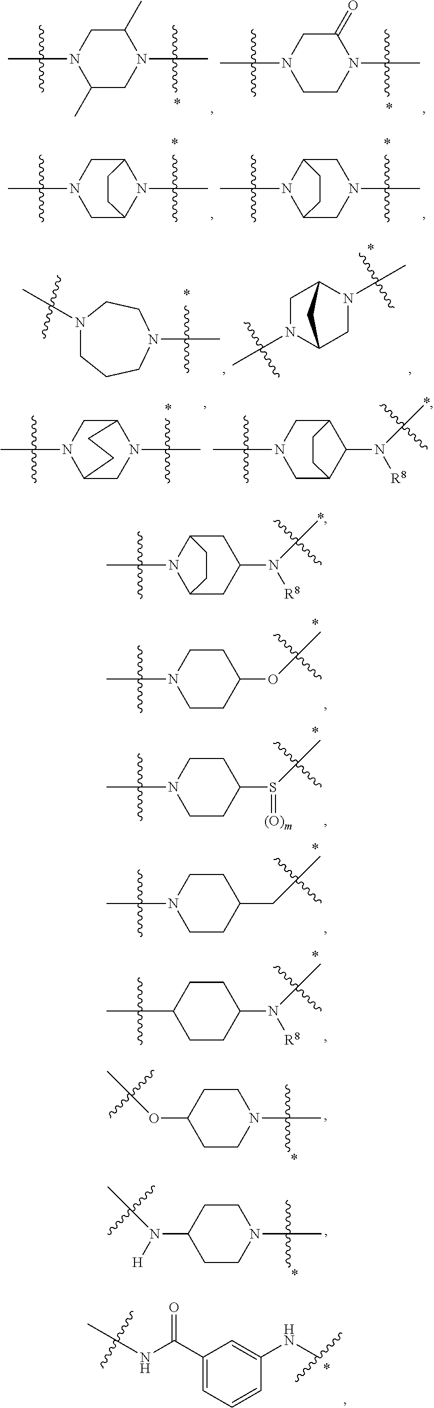

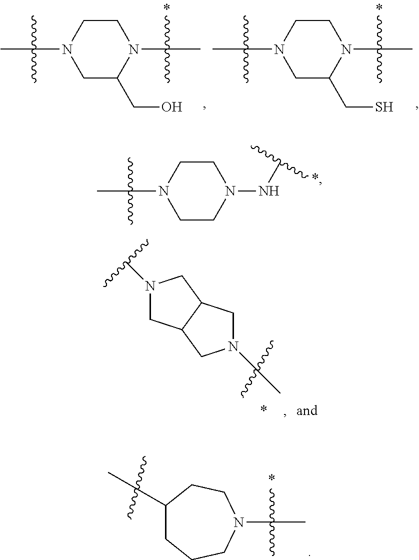 [SMILES: *N1CCN(*)C(CO)C1 ; *N1CCN(*)C(CS)C1 ; *NN1CCN(*)CC1 ; *N1CC2CN(*)CC2C1 ; *C1CCCN(*)CC1]

Another embodiment provides the compound of Formula (I), (Ia) or (Ib), or pharmaceutically acceptable salt or solvate thereof, wherein L is selected from:

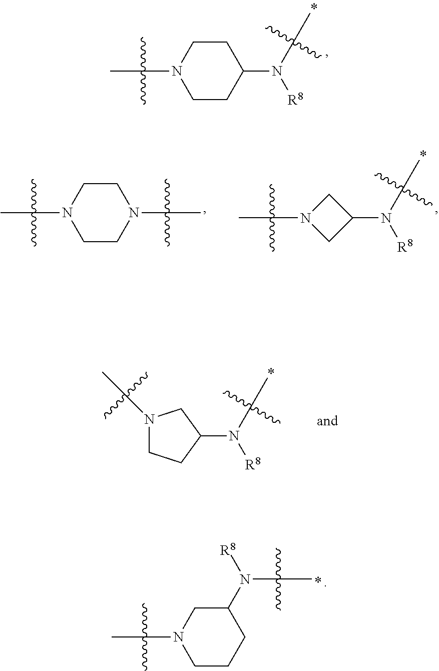

Another embodiment provides the compound of Formula (I), (Ia) or (Ib), or pharmaceutically acceptable salt or solvate thereof, wherein L is selected from:

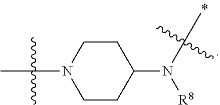

Another embodiment provides the compound of Formula (I), (Ia) or (Ib), or pharmaceutically acceptable salt or solvate thereof, wherein L is selected from:

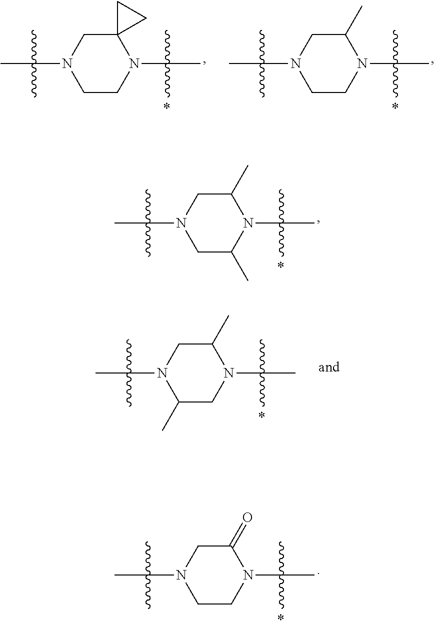

Another embodiment provides the compound of Formula (I), (Ia) or (Ib), or pharmaceutically acceptable salt or solvate thereof, wherein L is selected from:

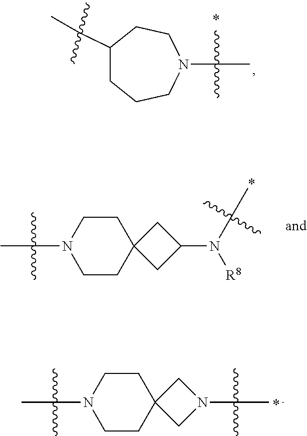

Another embodiment provides the compound of Formula (I), (Ia) or (Ib), or pharmaceutically acceptable salt or solvate thereof, wherein L is selected from:

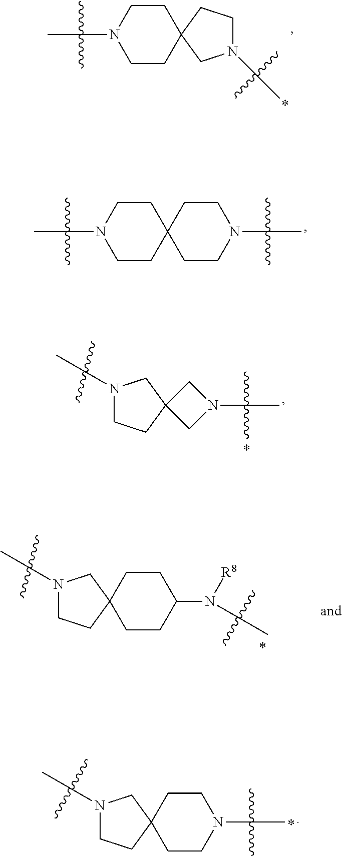

Another embodiment provides the compound of Formula (I), (Ia) or (Ib), or pharmaceutically acceptable salt or solvate thereof, wherein L is selected from:

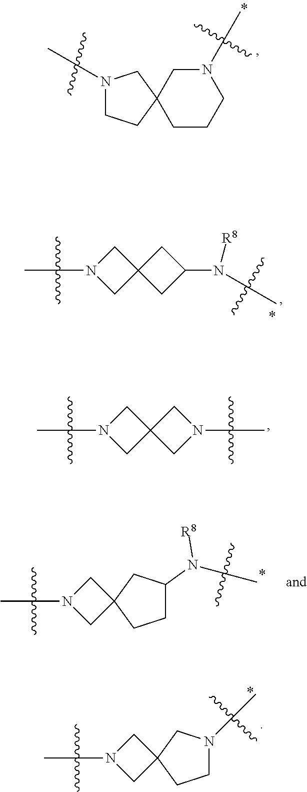

Another embodiment provides the compound of Formula (I), (Ia) or (Ib), or pharmaceutically acceptable salt or solvate thereof, wherein L is selected from:

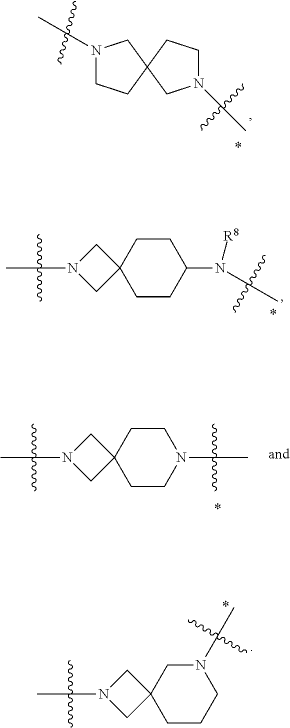

Another embodiment provides the compound of Formula (I), (Ia) or (Ib), or pharmaceutically acceptable salt or solvate thereof, wherein R⁸ is hydrogen. Another embodiment provides the compound of Formula (I), (Ia) or (Ib), or pharmaceutically acceptable salt or solvate thereof, wherein R⁸ is optionally substituted C1-C6 alkyl.

Another embodiment provides the compound of Formula (I), (Ia) or (Ib), or pharmaceutically acceptable salt or solvate thereof, wherein q is 0.

Another embodiment provides the compound of Formula (I), (Ia) or (Ib), or pharmaceutically acceptable salt or solvate thereof, wherein q is 1.

Another embodiment provides the compound of Formula (I), (Ia) or (Ib), or pharmaceutically acceptable salt or solvate thereof, wherein Ar is

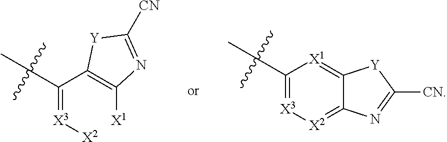

Another embodiment provides the compound of Formula (I), (Ia) or (Tb), or pharmaceutically acceptable salt or solvate thereof, wherein Ar is

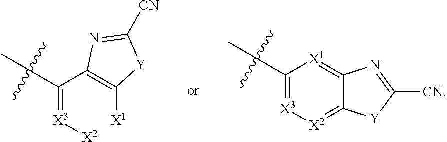

Another embodiment provides the compound of Formula (I), (Ia) or (Ib), or pharmaceutically acceptable salt or solvate thereof, wherein Ar is

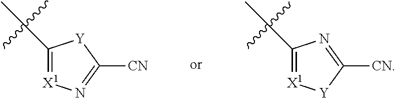

Another embodiment provides the compound of Formula (I), (Ia) or (Ib), or pharmaceutically acceptable salt or solvate thereof, wherein X¹, X², and X³ are C—H. Another embodiment provides the compound of Formula (I), (Ia) or (Ib), or pharmaceutically acceptable salt or solvate thereof, wherein X¹ is N; and X², and X³ are C—H. Another embodiment provides the compound of Formula (I), (Ia) or (Ib), or pharmaceutically acceptable salt or solvate thereof, wherein X² is N; and X¹, and X³ are C—H. Another embodiment provides the compound of Formula (I), (Ia) or (Ib), or pharmaceutically acceptable salt or solvate thereof, wherein X³ is N; and X¹, and X² are C—H. Another embodiment provides the compound of Formula (I), (Ia) or (Ib), or pharmaceutically acceptable salt or solvate thereof, wherein X¹ is N; and X², and X³ are C—H.

Another embodiment provides the compound of Formula (I), (Ia) or (Ib), or pharmaceutically acceptable salt or solvate thereof, wherein Y is O. Another embodiment provides the compound of Formula (I), (Ia) or (Ib), or pharmaceutically acceptable salt or solvate thereof, wherein Y is S. Another embodiment provides the compound of Formula (I), (Ia) or (Ib), or pharmaceutically acceptable salt or solvate thereof, wherein Y is N—R⁹. Another embodiment provides the compound of Formula (I), (Ia) or (Ib), or pharmaceutically acceptable salt or solvate thereof, wherein R⁹ is hydrogen. Another embodiment provides the compound of Formula (I), (Ia) or (Ib), or pharmaceutically acceptable salt or solvate thereof, wherein R⁹ is optionally substituted C1-C6 alkyl.

Another embodiment provides the compound of Formula (I), (Ia) or (Ib), or pharmaceutically acceptable salt or solvate thereof, wherein Ar is

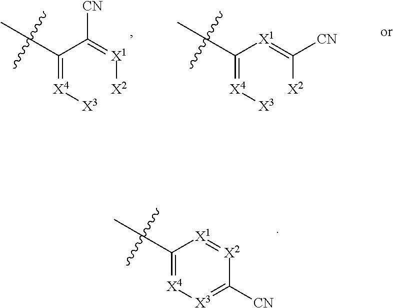

Another embodiment provides the compound of Formula (I), (Ia) or (Ib), or pharmaceutically acceptable salt or solvate thereof, wherein X¹ is N. Another embodiment provides the compound of Formula (I), (Ia) or (Ib), or pharmaceutically acceptable salt or solvate thereof, wherein X² is N. Another embodiment provides the compound of Formula (I), (Ia) or (Ib), or pharmaceutically acceptable salt or solvate thereof, wherein X³ is N. Another embodiment provides the compound of Formula (I), (Ia) or (Ib), or pharmaceutically acceptable salt or solvate thereof, wherein X⁴ is N. Another embodiment provides the compound of Formula (I), (Ia) or (Ib), or pharmaceutically acceptable salt or solvate thereof, wherein X¹ is C—H. Another embodiment provides the compound of Formula (I), (Ia) or (Ib), or pharmaceutically acceptable salt or solvate thereof, wherein X² is C—H. Another embodiment provides the compound of Formula (I), (Ia) or (Ib), or pharmaceutically acceptable salt or solvate thereof, wherein X³ is C—H. Another embodiment provides the compound of Formula (I), (Ia) or (Ib), or pharmaceutically acceptable salt or solvate thereof, wherein X⁴ is C—H.

Another embodiment provides the compound of Formula (I), (Ia) or (Ib), or pharmaceutically acceptable salt or solvate thereof, wherein Ar is

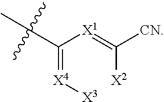

Another embodiment provides the compound of Formula (I), (Ia) or (Ib), or pharmaceutically acceptable salt or solvate thereof, wherein X¹ is N. Another embodiment provides the compound of Formula (I), (Ia) or (Ib), or pharmaceutically acceptable salt or solvate thereof, wherein X² is N. Another embodiment provides the compound of Formula (I), (Ia) or (Ib), or pharmaceutically acceptable salt or solvate thereof, wherein X³ is N. Another embodiment provides the compound of Formula (I), (Ia) or (Ib), or pharmaceutically acceptable salt or solvate thereof, wherein X⁴ is N. Another embodiment provides the compound of Formula (I), (Ia) or (Ib), or pharmaceutically acceptable salt or solvate thereof, wherein X¹ is C—H. Another embodiment provides the compound of Formula (I), (Ia) or (Ib), or pharmaceutically acceptable salt or solvate thereof, wherein X² is C—H. Another embodiment provides the compound of Formula (I), (Ia) or (Ib), or pharmaceutically acceptable salt or solvate thereof, wherein X³ is C—H. Another embodiment provides the compound of Formula (I), (Ia) or (Ib), or pharmaceutically acceptable salt or solvate thereof, wherein X⁴ is C—H.

Another embodiment provides the compound of Formula (I), (Ia) or (Ib), or pharmaceutically acceptable salt or solvate thereof, wherein R³ is optionally substituted C1-C6 alkyl. Another embodiment provides the compound of Formula (I), (Ia) or (Ib), or pharmaceutically acceptable salt or solvate thereof, wherein R³ is optionally substituted aryl.

Another embodiment provides the compound of Formula (I), (Ia) or (Ib), or pharmaceutically acceptable salt or solvate thereof, wherein R⁴ is optionally substituted C1-C6 alkyl. Another embodiment provides the compound of Formula (I), (Ia) or (Ib), or pharmaceutically acceptable salt or solvate thereof, wherein R⁴ is optionally substituted aryl.

One embodiment provides an AKT1 inhibitory compound, or a pharmaceutically acceptable salt or solvate thereof, having a structure presented in Table 1.

TABLE 1

| Example | Compound Structure | Compound Name |
|---|---|---|
| 1 | 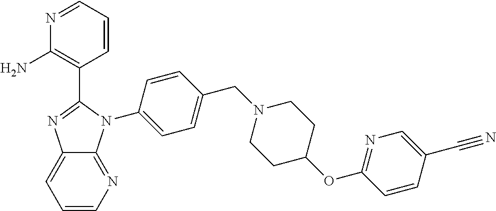 [SMILES: N#Cc1ccc(OC2CCN(Cc3ccc(-n4c(-c5cccnc5N)nc5cccnc54)cc3)CC2)nc1] | 6-((1-(4-(2-(2-aminopyridin-3-yl)-3H- imidazo[4,5-b]pyridin-3- yl)benzyl)piperidin-4-yl)oxy)nicotinonitrile |
| 2 | 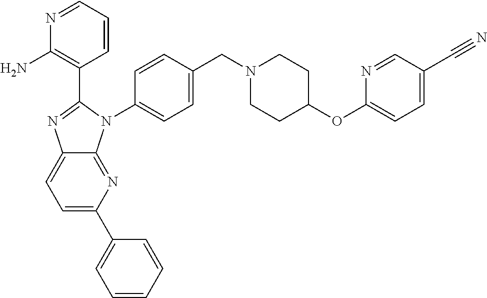 [SMILES: N#Cc1ccc(OC2CCN(Cc3ccc(-n4c(-c5cccnc5N)nc5ccc(-c6ccccc6)nc54)cc3)CC2)nc1] | 6-((1-(4-(2-(2-aminopyridin-3-yl)-5-phenyl- 3H-imidazo[4,5-b]pyridin-3- yl)benzyl)piperidin-4-yl)oxy)nicotinonitrile |
| 3 | 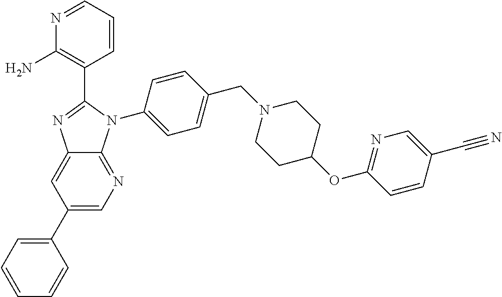 [SMILES: N#Cc1ccc(OC2CCN(Cc3ccc(-n4c(-c5cccnc5N)nc5cc(-c6ccccc6)cnc54)cc3)CC2)nc1] | 6-((1-(4-(2-(2-aminopyridin-3-yl)-6-phenyl- 3H-imidazo[4,5-b]pyridin-3- yl)benzyl)piperidin-4-yl)oxy)nicotinonitrile |
| 4 | 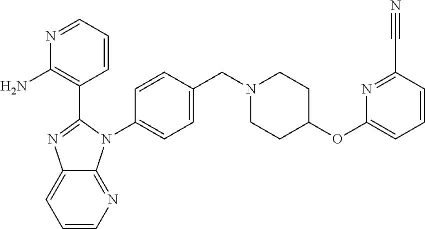 [SMILES: N#Cc1cccc(OC2CCN(Cc3ccc(-n4c(-c5cccnc5N)nc5cccnc54)cc3)CC2)n1] | 6-((1-(4-(2-(2-aminopyridin-3-yl)-3H- imidazo[4,5-b]pyridin-3- yl)benzyl)piperidin-4-yl)oxy)picolinonitrile |
| 5 | 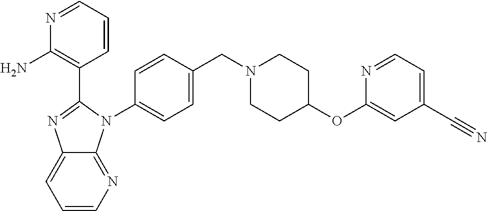 [SMILES: N#Cc1ccnc(OC2CCN(Cc3ccc(-n4c(-c5cccnc5N)nc5cccnc54)cc3)CC2)c1] | 2-((1-(4-(2-(2-aminopyridin-3-yl)-3H- imidazo[4,5-b]pyridin-3- yl)benzyl)piperidin-4- yl)oxy)isonicotinonitrile |
| 6 | 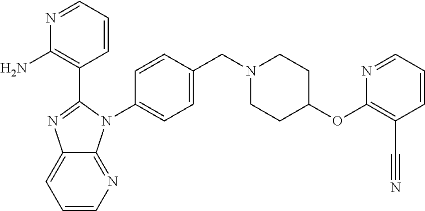 [SMILES: N#Cc1cccnc1OC1CCN(Cc2ccc(-n3c(-c4cccnc4N)nc4cccnc43)cc2)CC1] | 2-((1-(4-(2-(2-aminopyridin-3-yl)-3H- imidazo[4,5-b]pyridin-3- yl)benzyl)piperidin-4-yl)oxy)nicotinonitrile |
| 7 | 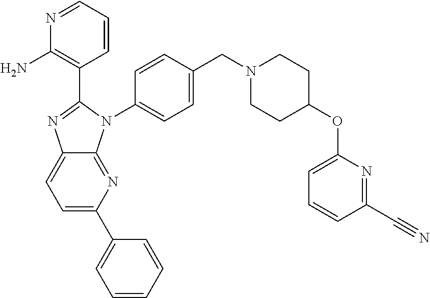 [SMILES: N#Cc1cccc(OC2CCN(Cc3ccc(-n4c(-c5cccnc5N)nc5ccc(-c6ccccc6)nc54)cc3)CC2)n1] | 6-((1-(4-(2-(2-aminopyridin-3-yl)-5-phenyl- 3H-imidazo[4,5-b]pyridin-3- yl)benzyl)piperidin-4-yl)oxy)picolinonitrile |
| 8 | 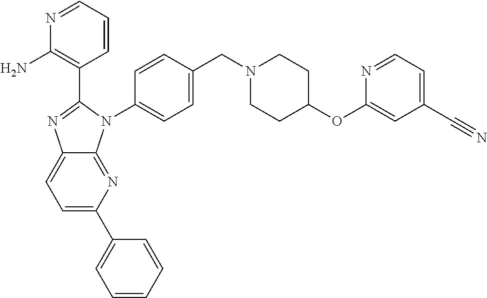 [SMILES: N#Cc1ccnc(OC2CCN(Cc3ccc(-n4c(-c5cccnc5N)nc5ccc(-c6ccccc6)nc54)cc3)CC2)c1] | 2-((1-(4-(2-(2-aminopyridin-3-yl)-5-phenyl- 3H-imidazo[4,5-b]pyridin-3- yl)benzyl)piperidin-4- yl)oxy)isonicotinonitrile |
| 9 | 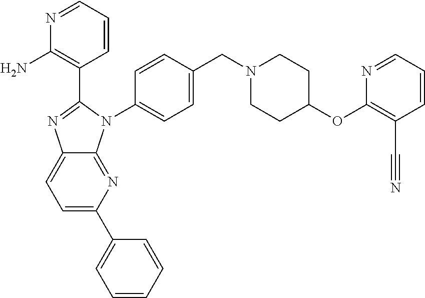 [SMILES: N#Cc1cccnc1OC1CCN(Cc2ccc(-n3c(-c4cccnc4N)nc4ccc(-c5ccccc5)nc43)cc2)CC1] | 2-((1-(4-(2-(2-aminopyridin-3-yl)-5-phenyl- 3H-imidazo[4,5-b]pyridin-3- yl)benzyl)piperidin-4-yl)oxy)nicotinonitrile |
| 10 | 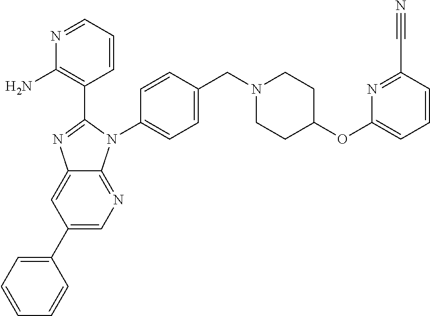 [SMILES: N#Cc1cccc(OC2CCN(Cc3ccc(-n4c(-c5cccnc5N)nc5cc(-c6ccccc6)cnc54)cc3)CC2)n1] | 6-((1-(4-(2-(2-aminopyridin-3-yl)-6-phenyl- 3H-imidazo[4,5-b]pyridin-3- yl)benzyl)piperidin-4-yl)oxy)picolinonitrile |
| 11 | 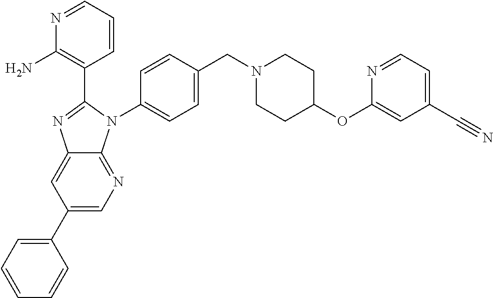 [SMILES: N#Cc1ccnc(OC2CCN(Cc3ccc(-n4c(-c5cccnc5N)nc5cc(-c6ccccc6)cnc54)cc3)CC2)c1] | 2-((1-(4-(2-(2-aminopyridin-3-yl)-6-phenyl- 3H-imidazo[4,5-b ]pyridin-3- yl)benzyl)piperidin-4- yl)oxy)isonicotinonitrile |
| 12 | 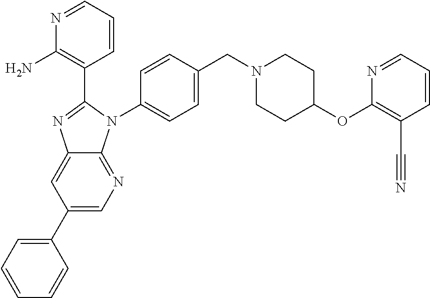 [SMILES: N#Cc1cccnc1OC1CCN(Cc2ccc(-n3c(-c4cccnc4N)nc4cc(-c5ccccc5)cnc43)cc2)CC1] | 2-((1-(4-(2-(2-aminopyridin-3-yl)-6-phenyl- 3H-imidazo[4,5-b]pyridin-3- yl)benzyl)piperidin-4-yl)oxy)nicotinonitrile |
| 13 | 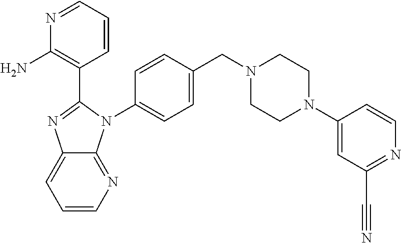 [SMILES: N#Cc1cc(N2CCN(Cc3ccc(-n4c(-c5cccnc5N)nc5cccnc54)cc3)CC2)ccn1] | 4-(4-(4-(2-(2-aminopyridin-3-yl)-3H- imidazo[4,5-b]pyridin-3- yl)benzyl)piperazin-1-yl)picolinonitrile |
| 14 | 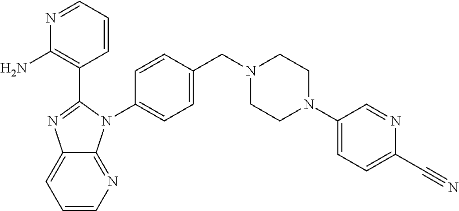 [SMILES: N#Cc1ccc(N2CCN(Cc3ccc(-n4c(-c5cccnc5N)nc5cccnc54)cc3)CC2)cn1] | 5-(4-(4-(2-(2-aminopyridin-3-yl)-3H- imidazo[4,5-b]pyridin-3- yl)benzyl)piperazin-1-yl)picolinonitrile |
| 15 | 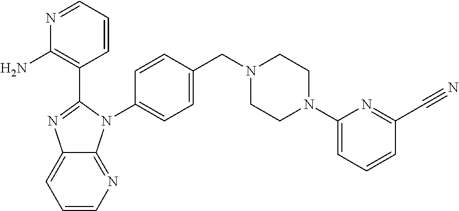 [SMILES: N#Cc1cccc(N2CCN(Cc3ccc(-n4c(-c5cccnc5N)nc5cccnc54)cc3)CC2)n1] | 6-(4-(4-(2-(2-aminopyridin-3-yl)-3H- imidazo[4,5-b]pyridin-3- yl)benzyl)piperazin-1-yl)picolinonitrile |
| 16 | 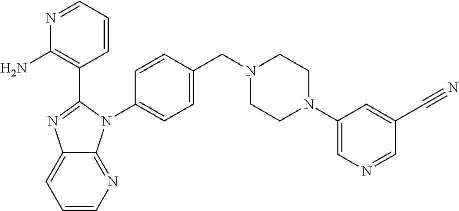 [SMILES: N#Cc1cncc(N2CCN(Cc3ccc(-n4c(-c5cccnc5N)nc5cccnc54)cc3)CC2)c1] | 5-(4-(4-(2-(2-aminopyridin-3-yl)-3H- imidazo[4,5-b]pyridin-3- yl)benzyl)piperazin-1-yl)nicotinonitrile |
| 17 | 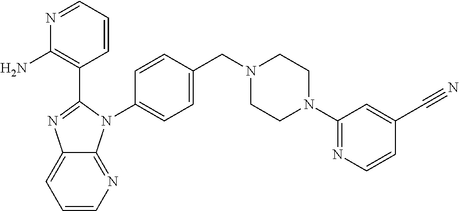 [SMILES: N#Cc1ccnc(N2CCN(Cc3ccc(-n4c(-c5cccnc5N)nc5cccnc54)cc3)CC2)c1] | 2-(4-(4-(2-(2-aminopyridin-3-yl)-3H- imidazo[4,5-b]pyridin-3- yl)benzyl)piperazin-1-yl)isonicotinonitrile |
| 18 | 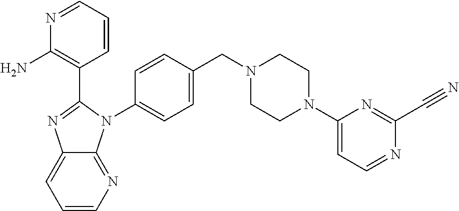 [SMILES: N#Cc1nccc(N2CCN(Cc3ccc(-n4c(-c5cccnc5N)nc5cccnc54)cc3)CC2)n1] | 4-(4-(4-(2-(2-aminopyridin-3-yl)-3H- imidazo[4,5-b]pyridin-3- yl)benzyl)piperazin-1-yl)pyrimidine-2- carbonitrile |
| 19 | 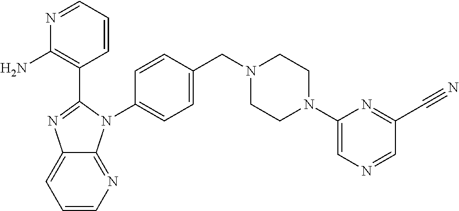 [SMILES: N#Cc1cncc(N2CCN(Cc3ccc(-n4c(-c5cccnc5N)nc5cccnc54)cc3)CC2)n1] | 6-(4-(4-(2-(2-aminopyridin-3-yl)-3H- imidazo[4,5-b]pyridin-3- yl)benzyl)piperazin-1-yl)pyrazine-2- carbonitrile |
| 20 | 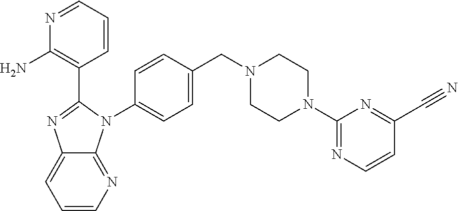 [SMILES: N#Cc1ccnc(N2CCN(Cc3ccc(-n4c(-c5cccnc5N)nc5cccnc54)cc3)CC2)n1] | 2-(4-(4-(2-(2-aminopyridin-3-yl)-3H- imidazo[4,5-b]pyridin-3- yl)benzyl)piperazin-1-yl)pyrimidine-4- carbonitrile |
| 21 | 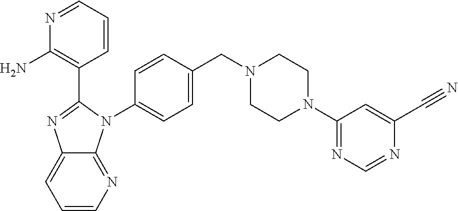 [SMILES: N#Cc1cc(N2CCN(Cc3ccc(-n4c(-c5cccnc5N)nc5cccnc54)cc3)CC2)ncn1] | 6-(4-(4-(2-(2-aminopyridin-3-yl)-3H- imidazo[4,5-b]pyridin-3- yl)benzyl)piperazin-1-yl)pyrimidine-4- carbonitrile |
| 22 | 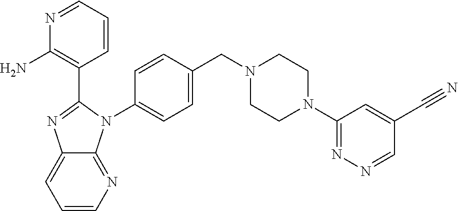 [SMILES: N#Cc1cnnc(N2CCN(Cc3ccc(-n4c(-c5cccnc5N)nc5cccnc54)cc3)CC2)c1] | 6-(4-(4-(2-(2-aminopyridin-3-yl)-3H- imidazo[4,5-b]pyridin-3- yl)benzyl)piperazin-1-yl)pyridazine-4- carbonitrile |
| 23 | 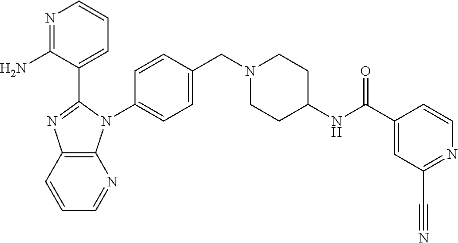 [SMILES: N#Cc1cc(C(=O)NC2CCN(Cc3ccc(-n4c(-c5cccnc5N)nc5cccnc54)cc3)CC2)ccn1] | N-(1-(4-(2-(2-aminopyridin-3-yl)-3H- imidazo[4,5-b]pyridin-3- yl)benzyl)piperidin-4-yl)-2- cyanoisonicotinamide |
| 24 | 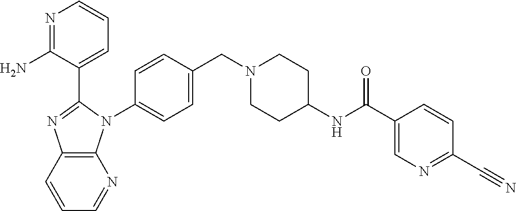 [SMILES: N#Cc1ccc(C(=O)NC2CCN(Cc3ccc(-n4c(-c5cccnc5N)nc5cccnc54)cc3)CC2)cn1] | N-(1-(4-(2-(2-aminopyridin-3-yl)-3H- imidazo[4,5-b]pyridin-3- yl)benzyl)piperidin-4-yl)-6- cyanonicotinamide |
| 25 | 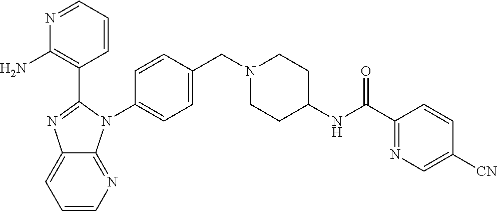 [SMILES: N#Cc1ccc(C(=O)NC2CCN(Cc3ccc(-n4c(-c5cccnc5N)nc5cccnc54)cc3)CC2)nc1] | N-(1-(4-(2-(2-aminopyridin-3-yl)-3H- imidazo[4,5-b]pyridin-3- yl)benzyl)piperidin-4-yl)-5- cyanopicolinamide |
| 26 | 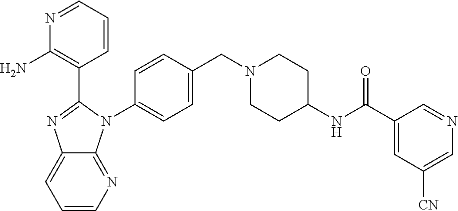 [SMILES: N#Cc1cncc(C(=O)NC2CCN(Cc3ccc(-n4c(-c5cccnc5N)nc5cccnc54)cc3)CC2)c1] | N-(1-(4-(2-(2-aminopyridin-3-yl)-3H- imidazo[4,5-b]pyridin-3- yl)benzyl)piperidin-4-yl)-5- cyanonicotinamide |
| 27 | 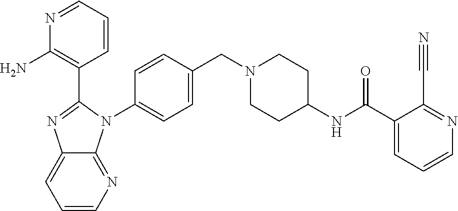 [SMILES: N#Cc1ncccc1C(=O)NC1CCN(Cc2ccc(-n3c(-c4cccnc4N)nc4cccnc43)cc2)CC1] | N-(1-(4-(2-(2-aminopyridin-3-yl)-3H- imidazo[4,5-b]pyridin-3- yl)benzyl)piperidin-4-yl)-2- cyanonicotinamide |
| 28 | 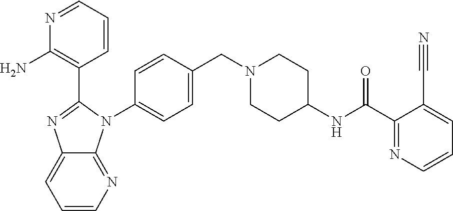 [SMILES: N#Cc1cccnc1C(=O)NC1CCN(Cc2ccc(-n3c(-c4cccnc4N)nc4cccnc43)cc2)CC1] | N-(1-(4-(2-(2-aminopyridin-3-yl)-3H- imidazo[4,5-b]pyridin-3- yl)benzyl)piperidin-4-yl)-3- cyanopicolinamide |
| 29 | 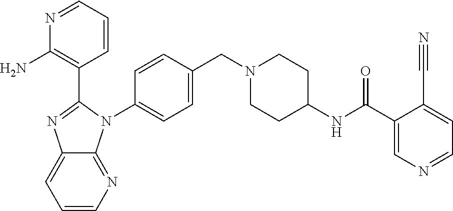 [SMILES: N#Cc1ccncc1C(=O)NC1CCN(Cc2ccc(-n3c(-c4cccnc4N)nc4cccnc43)cc2)CC1] | N-(1-(4-(2-(2-aminopyridin-3-yl)-3H- imidazo[4,5-b]pyridin-3- yl)benzyl)piperidin-4-yl)-4- cyanonicotinamide |
| 30 | 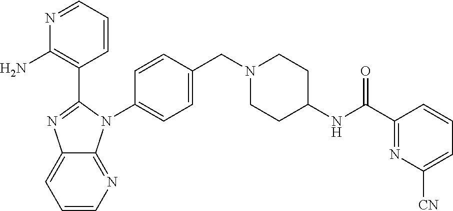 [SMILES: N#Cc1cccc(C(=O)NC2CCN(Cc3ccc(-n4c(-c5cccnc5N)nc5cccnc54)cc3)CC2)n1] | N-(1-(4-(2-(2-aminopyridin-3-yl)-3H- imidazo[4,5-b]pyridin-3- yl)benzyl)piperidin-4-yl)-6- cyanopicolinamide |
| 31 | 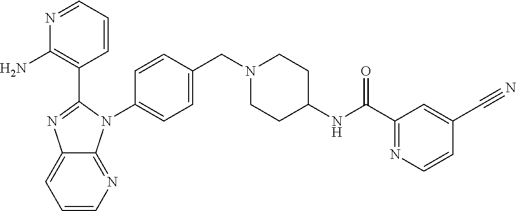 [SMILES: N#Cc1ccnc(C(=O)NC2CCN(Cc3ccc(-n4c(-c5cccnc5N)nc5cccnc54)cc3)CC2)c1] | N-(1-(4-(2-(2-aminopyridin-3-yl)-3H- imidazo[4,5-b]pyridin-3- yl)benzyl)piperidin-4-yl)-4- cyanopicolinamide |
| 32 | 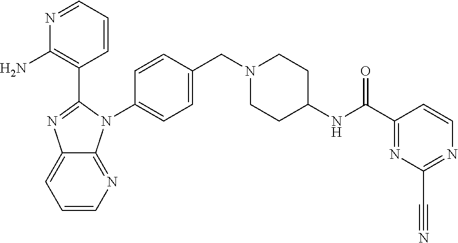 [SMILES: N#Cc1nccc(C(=O)NC2CCN(Cc3ccc(-n4c(-c5cccnc5N)nc5cccnc54)cc3)CC2)n1] | N-(1-(4-(2-(2-aminopyridin-3-yl)-3H- imidazo[4,5-b]pyridin-3- yl)benzyl)piperidin-4-yl)-2- cyanopyrimidine-4-carboxamide |
| 33 | 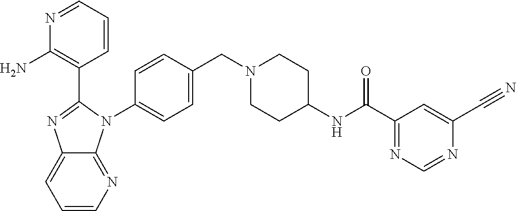 [SMILES: N#Cc1cc(C(=O)NC2CCN(Cc3ccc(-n4c(-c5cccnc5N)nc5cccnc54)cc3)CC2)ncn1] | N-(1-(4-(2-(2-aminopyridin-3-yl)-3H- imidazo[4,5-b]pyridin-3- yl)benzyl)piperidin-4-yl)-6- cyanopyrimidine-4-carboxamide |
| 34 | 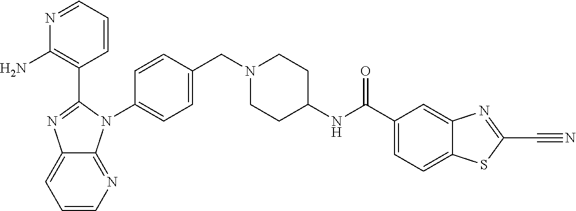 [SMILES: N#Cc1nc2cc(C(=O)NC3CCN(Cc4ccc(-n5c(-c6cccnc6N)nc6cccnc65)cc4)CC3)ccc2s1] | N-(1-(4-(2-(2-aminopyridin-3-yl)-3H- imidazo[4,5-b]pyridin-3- yl)benzyl)piperidin-4-yl)-2- cyanobenzo[d]thiazole-5-carboxamide |
| 35 | 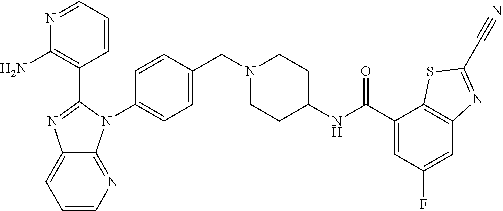 [SMILES: N#Cc1nc2cc(F)cc(C(=O)NC3CCN(Cc4ccc(-n5c(-c6cccnc6N)nc6cccnc65)cc4)CC3)c2s1] | N-(1-(4-(2-(2-aminopyridin-3-yl)-3H- imidazo[4,5-b]pyridin-3- yl)benzyl)piperidin-4-yl)-2-cyano-5- fluorobenzo[d]thiazole-7-carboxamide |
| 36 | 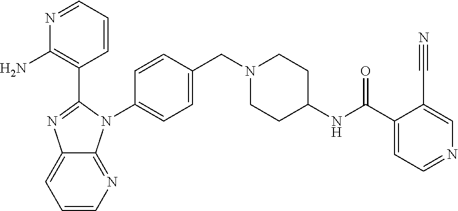 [SMILES: N#Cc1cnccc1C(=O)NC1CCN(Cc2ccc(-n3c(-c4cccnc4N)nc4cccnc43)cc2)CC1] | N-(1-(4-(2-(2-aminopyridin-3-yl)-3H- imidazo[4,5-b]pyridin-3- yl)benzyl)piperidin-4-yl)-3- cyanoisonicotinamide |
| 37 | 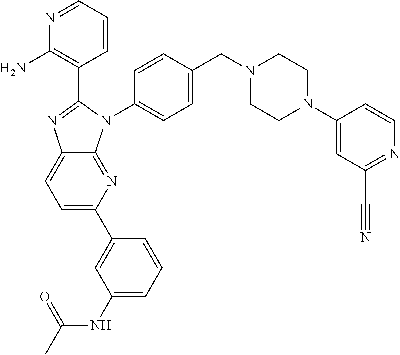 [SMILES: CC(=O)Nc1cccc(-c2ccc3nc(-c4cccnc4N)n(-c4ccc(CN5CCN(c6ccnc(C#N)c6)CC5)cc4)c3n2)c1] | N-(3-(2-(2-aminopyridin-3-yl)-3-(4-((4-(2- cyanopyridin-4-yl)piperazin-1- yl)methyl)phenyl)-3H-imidazo[4,5- b]pyridin-5-yl)phenyl)acetamide |
| 38 | 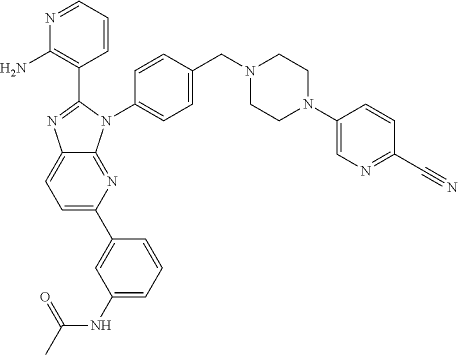 [SMILES: CC(=O)Nc1cccc(-c2ccc3nc(-c4cccnc4N)n(-c4ccc(CN5CCN(c6ccc(C#N)nc6)CC5)cc4)c3n2)c1] | N-(3-(2-(2-aminopyridin-3-yl)-3-(4-((4-(6- cyanopyridin-3-yl)piperazin-1- yl)methyl)phenyl)-3H-imidazo[4,5- b]pyridin-5-yl)phenyl)acetamide |
| 39 | 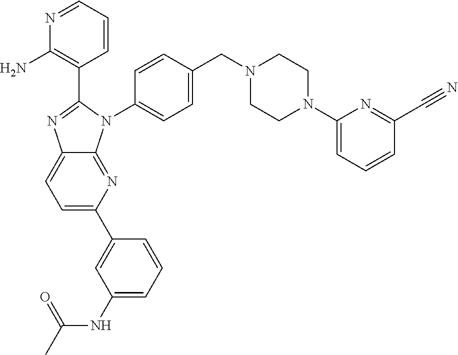 [SMILES: CC(=O)Nc1cccc(-c2ccc3nc(-c4cccnc4N)n(-c4ccc(CN5CCN(c6cccc(C#N)n6)CC5)cc4)c3n2)c1] | N-(3-(2-(2-aminopyridin-3-yl)-3-(4-((4-(6- cyanopyridin-2-yl)piperazin-1- yl)methyl)phenyl)-3H-imidazo[4,5- b]pyridin-5-yl)phenyl)acetamide |
| 40 | 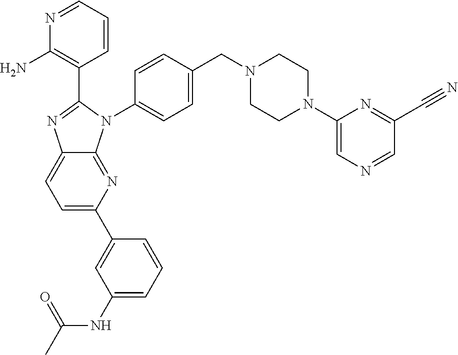 [SMILES: CC(=O)Nc1cccc(-c2ccc3nc(-c4cccnc4N)n(-c4ccc(CN5CCN(c6cncc(C#N)n6)CC5)cc4)c3n2)c1] | N-(3-(2-(2-aminopyridin-3-yl)-3-(4-((4-(6- cyanopyrazin-2-yl)piperazin-1- yl)methyl)phenyl)-3H-imidazo[4,5- b]pyridin-5-yl)phenyl)acetamide |
| 41 | 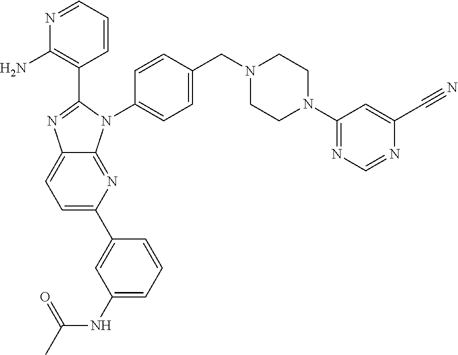 [SMILES: CC(=O)Nc1cccc(-c2ccc3nc(-c4cccnc4N)n(-c4ccc(CN5CCN(c6cc(C#N)ncn6)CC5)cc4)c3n2)c1] | N-(3-(2-(2-aminopyridin-3-yl)-3-(4-((4-(6- cyanopyrimidin-4-yl)piperazin-1- yl)methyl)phenyl)-3H-imidazo[4,5- b]pyridin-5-yl)phenyl)acetamide |
| 42 | 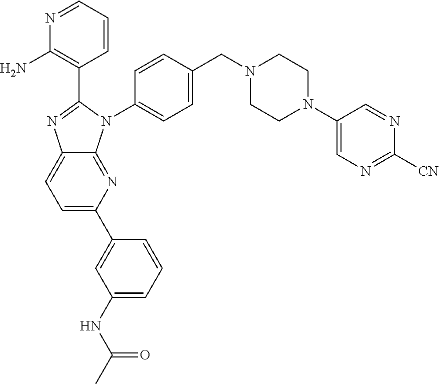 [SMILES: CC(=O)Nc1cccc(-c2ccc3nc(-c4cccnc4N)n(-c4ccc(CN5CCN(c6cnc(C#N)nc6)CC5)cc4)c3n2)c1] | N-(3-(2-(2-aminopyridin-3-yl)-3-(4-((4-(2- cyanopyrimidin-5-yl)piperazin-1- yl)methyl)phenyl)-3H-imidazo[4,5- b]pyridin-5-yl)phenyl)acetamide |
| 43 | 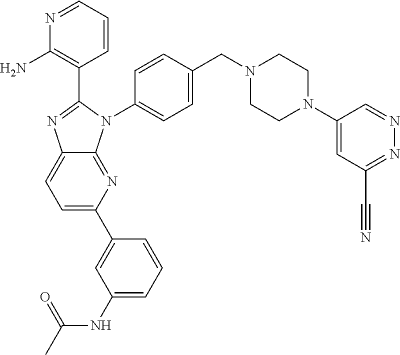 [SMILES: CC(=O)Nc1cccc(-c2ccc3nc(-c4cccnc4N)n(-c4ccc(CN5CCN(c6cnnc(C#N)c6)CC5)cc4)c3n2)c1] | N-(3-(2-(2-aminopyridin-3-yl)-3-(4-((4-(6- cyanopyridazin-4-yl)piperazin-1- yl)methyl)phenyl)- 3H-imidazo[4,5- b]pyridin-5-yl)phenyl)acetamide |
| 44 | 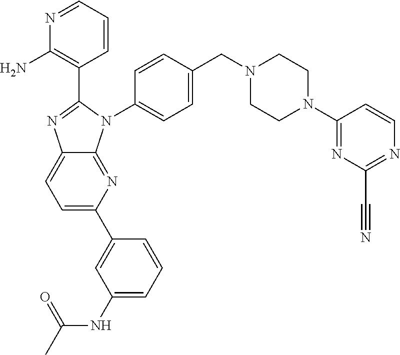 [SMILES: CC(=O)Nc1cccc(-c2ccc3nc(-c4cccnc4N)n(-c4ccc(CN5CCN(c6ccnc(C#N)n6)CC5)cc4)c3n2)c1] | N-(3-(2-(2-aminopyridin-3-yl)-3-(4-((4-(2- cyanopyrimidin-4-yl)piperazin-1- yl)methyl)phenyl)-3H-imidazo[4,5- b]pyridin-5-yl)phenyl)acetamide |
| 45 | 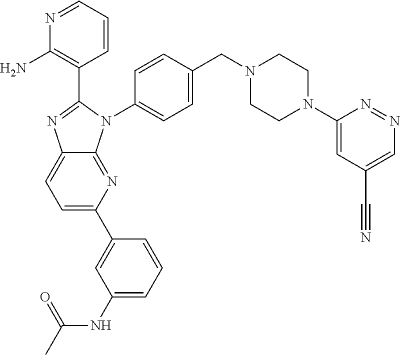 [SMILES: CC(=O)Nc1cccc(-c2ccc3nc(-c4cccnc4N)n(-c4ccc(CN5CCN(c6cc(C#N)cnn6)CC5)cc4)c3n2)c1] | N-(3-(2-(2-aminopyridin-3-yl)-3-(4-((4-(5- cyanopyridazin-3-yl)piperazin-1- yl)methyl)phenyl)-3H-imidazo[4,5- b]pyridin-5-yl)phenyl)acetamide |
| 46 | 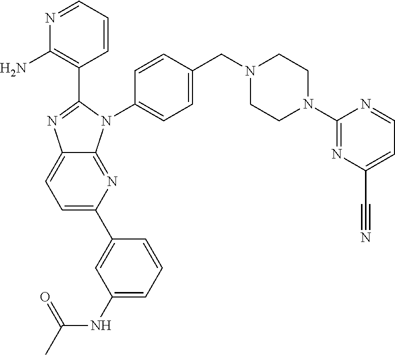 [SMILES: CC(=O)Nc1cccc(-c2ccc3nc(-c4cccnc4N)n(-c4ccc(CN5CCN(c6nccc(C#N)n6)CC5)cc4)c3n2)c1] | N-(3-(2-(2-aminopyridin-3-yl)-3-(4-((4-(4- cyanopyrimidin-2-yl)piperazin-1- yl)methyl)phenyl)-3H-imidazo[4,5- b]pyridin-5-yl)phenyl)acetamide |
| 47 | 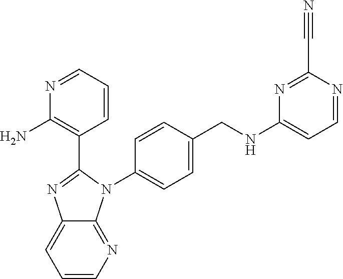 [SMILES: N#Cc1nccc(NCc2ccc(-n3c(-c4cccnc4N)nc4cccnc43)cc2)n1] | 4-((4-(2-(2-aminopyridin-3-yl)-3H- imidazo[4,5-b]pyridin-3- yl)benzyl)amino)pyrimidine-2-carbonitrile |
| 48 | 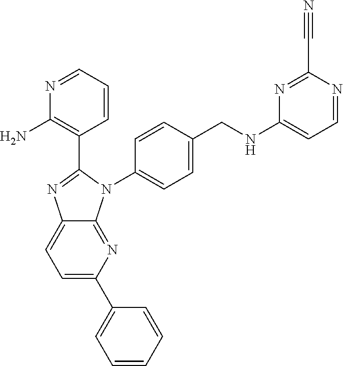 [SMILES: N#Cc1nccc(NCc2ccc(-n3c(-c4cccnc4N)nc4ccc(-c5ccccc5)nc43)cc2)n1] | 4-((4-(2-(2-aminopyridin-3-yl)-5-phenyl- 3H-imidazo[4,5-b]pyridin-3- yl)benzyl)amino)pyrimidine-2-carbonitrile |
| 49 | 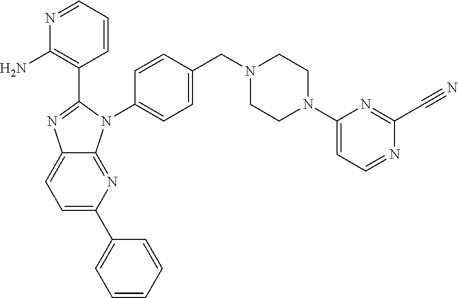 [SMILES: N#Cc1nccc(N2CCN(Cc3ccc(-n4c(-c5cccnc5N)nc5ccc(-c6ccccc6)nc54)cc3)CC2)n1] | 4-(4-(4-(2-(2-aminopyridin-3-yl)-5-phenyl- 3H-imidazo[4,5-b ]pyridin-3- yl)benzyl)piperazin-1-yl)pyrimidine-2- carbonitrile |
| 50 | 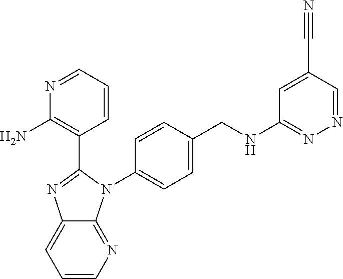 [SMILES: N#Cc1cnnc(NCc2ccc(-n3c(-c4cccnc4N)nc4cccnc43)cc2)c1] | 6-((4-(2-(2-aminopyridin-3-yl)-3H- imidazo[4,5-b]pyridin-3- yl)benzyl)amino)pyridazine-4-carbonitrile |
| 51 | 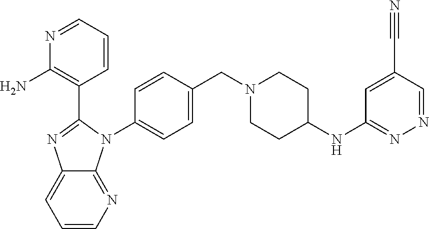 [SMILES: N#Cc1cnnc(NC2CCN(Cc3ccc(-n4c(-c5cccnc5N)nc5cccnc54)cc3)CC2)c1] | 6-((1-(4-(2-(2-aminopyridin-3-yl)-3H- imidazo[4,5-b]pyridin-3- yl)benzyl)piperidin-4-yl)amino)pyridazine- 4-carbonitrile |
| 52 | 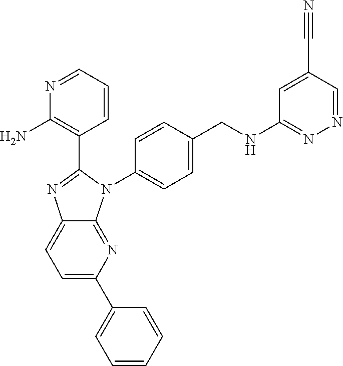 [SMILES: N#Cc1cnnc(NCc2ccc(-n3c(-c4cccnc4N)nc4ccc(-c5ccccc5)nc43)cc2)c1] | 6-((4-(2-(2-aminopyridin-3-yl)-5-phenyl- 3H-imidazo[4,5-b]pyridin-3- yl)benzyl)amino)pyridazine-4-carbonitrile |
| 53 | 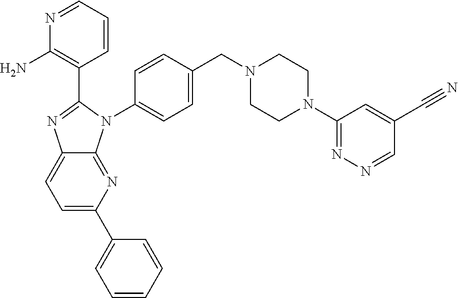 [SMILES: N#Cc1cnnc(N2CCN(Cc3ccc(-n4c(-c5cccnc5N)nc5ccc(-c6ccccc6)nc54)cc3)CC2)c1] | 6-(4-(4-(2-(2-aminopyridin-3-yl)-5-phenyl- 3H-imidazo[4,5-b]pyridin-3- yl)benzyl)piperazin-1-yl)pyridazine-4- carbonitrile |
| 54 | 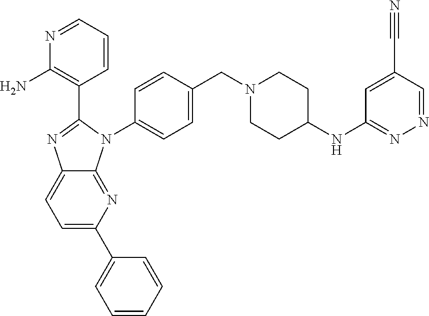 [SMILES: N#Cc1cnnc(NC2CCN(Cc3ccc(-n4c(-c5cccnc5N)nc5ccc(-c6ccccc6)nc54)cc3)CC2)c1] | 6-((1-(4-(2-(2-aminopyridin-3-yl)-5-phenyl- 3H-imidazo[4,5-b]pyridin-3- yl)benzyl)piperidin-4-yl)amino)pyridazine- 4-carbonitrile |
| 55 | 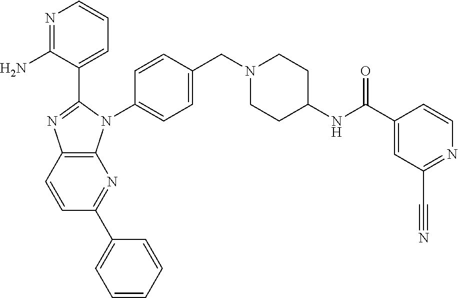 [SMILES: N#Cc1cc(C(=O)NC2CCN(Cc3ccc(-n4c(-c5cccnc5N)nc5ccc(-c6ccccc6)nc54)cc3)CC2)ccn1] | N-(1-(4-(2-(2-aminopyridin-3-yl)-5-phenyl- 3H-imidazo[4,5-b]pyridin-3- yl)benzyl)piperidin-4-yl)-2- cyanoisonicotinamide |
| 56 | 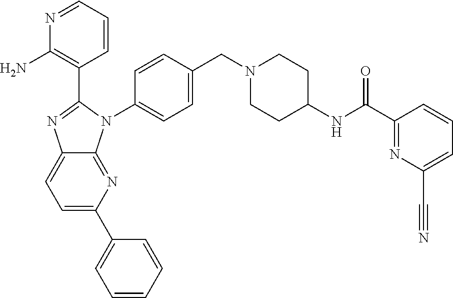 [SMILES: N#Cc1cccc(C(=O)NC2CCN(Cc3ccc(-n4c(-c5cccnc5N)nc5ccc(-c6ccccc6)nc54)cc3)CC2)n1] | N-(1-(4-(2-(2-aminopyridin-3-yl)-5-phenyl- 3H-imidazo[4,5-b]pyridin-3- yl)benzyl)piperidin-4-yl)-6- cyanopicolinamide |
| 57 | 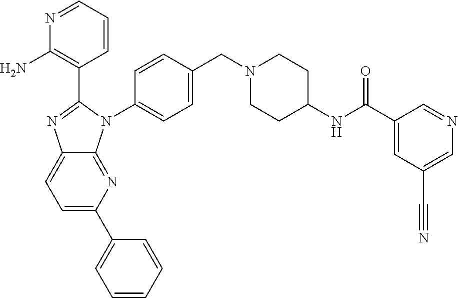 [SMILES: N#Cc1cncc(C(=O)NC2CCN(Cc3ccc(-n4c(-c5cccnc5N)nc5ccc(-c6ccccc6)nc54)cc3)CC2)c1] | N-(1-(4-(2-(2-aminopyridin-3-yl)-5-phenyl- 3H-imidazo[4,5-b]pyridin-3- yl)benzyl)piperidin-4-yl)-5- cyanonicotinamide |
| 58 | 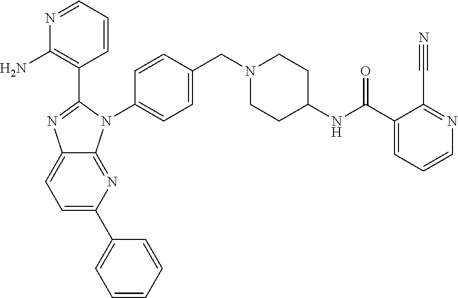 [SMILES: N#Cc1ncccc1C(=O)NC1CCN(Cc2ccc(-n3c(-c4cccnc4N)nc4ccc(-c5ccccc5)nc43)cc2)CC1] | N-(1-(4-(2-(2-aminopyridin-3-yl)-5-phenyl- 3H-imidazo[4,5-b ]pyridin-3- yl)benzyl)piperidin-4-yl)-2- cyanonicotinamide |
| 59 | 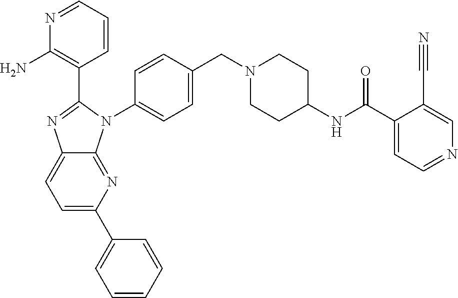 [SMILES: N#Cc1cnccc1C(=O)NC1CCN(Cc2ccc(-n3c(-c4cccnc4N)nc4ccc(-c5ccccc5)nc43)cc2)CC1] | N-(1-(4-(2-(2-aminopyridin-3-yl)-5-phenyl- 3H-imidazo[4,5-b]pyridin-3- yl)benzyl)piperidin-4-yl)-3- cyanoisonicotinamide |
| 60 | 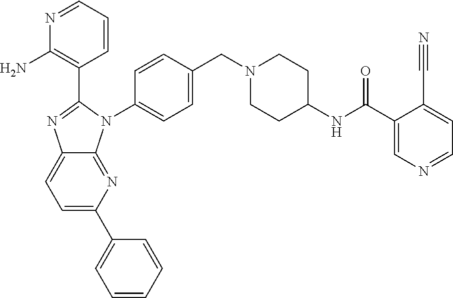 [SMILES: N#Cc1ccncc1C(=O)NC1CCN(Cc2ccc(-n3c(-c4cccnc4N)nc4ccc(-c5ccccc5)nc43)cc2)CC1] | N-(1-(4-(2-(2-aminopyridin-3-yl)-5-phenyl- 3H-imidazo[4,5-b]pyridin-3- yl)benzyl)piperidin-4-yl)-4- cyanonicotinamide |
| 61 | 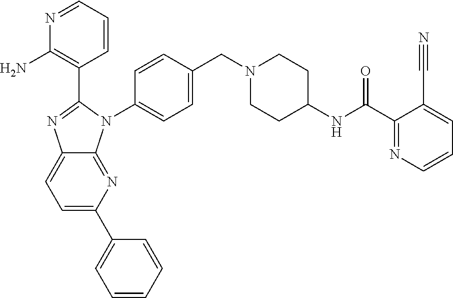 [SMILES: N#Cc1cccnc1C(=O)NC1CCN(Cc2ccc(-n3c(-c4cccnc4N)nc4ccc(-c5ccccc5)nc43)cc2)CC1] | N-(1-(4-(2-(2-aminopyridin-3-yl)-5-phenyl- 3H-imidazo[4,5-b]pyridin-3- yl)benzyl)piperidin-4-yl)-3- cyanopicolinamide |
| 62 | 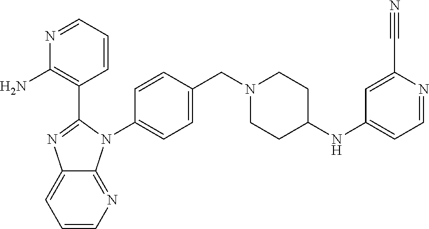 [SMILES: N#Cc1cc(NC2CCN(Cc3ccc(-n4c(-c5cccnc5N)nc5cccnc54)cc3)CC2)ccn1] | 4-((1-(4-(2-(2-aminopyridin-3-yl)-3H- imidazo[4,5-b]pyridin-3- yl)benzyl)piperidin-4- yl)amino)picolinonitrile |
| 63 | 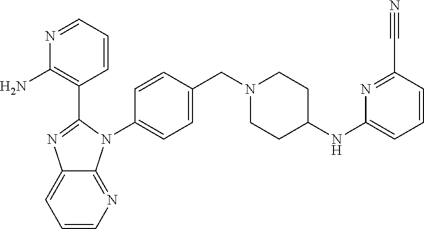 [SMILES: N#Cc1cccc(NC2CCN(Cc3ccc(-n4c(-c5cccnc5N)nc5cccnc54)cc3)CC2)n1] | 6-((1-(4-(2-(2-aminopyridin-3-yl)-3H- imidazo[4,5-b]pyridin-3- yl)benzyl)piperidin-4- yl)amino)picolinonitrile |
| 64 | 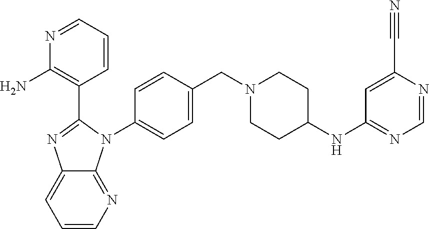 [SMILES: N#Cc1cc(NC2CCN(Cc3ccc(-n4c(-c5cccnc5N)nc5cccnc54)cc3)CC2)ncn1] | 6-((1-(4-(2-(2-aminopyridin-3-yl)-3H- imidazo[4,5-b]pyridin-3- yl)benzyl)piperidin-4-yl)amino)pyrimidine- 4-carbonitrile |
| 65 | 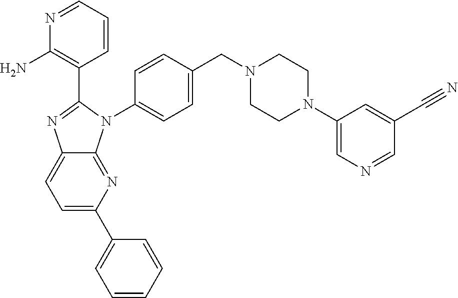 [SMILES: N#Cc1cncc(N2CCN(Cc3ccc(-n4c(-c5cccnc5N)nc5ccc(-c6ccccc6)nc54)cc3)CC2)c1] | 5-(4-(4-(2-(2-aminopyridin-3-yl)-5-phenyl- 3H-imidazo[4,5-b]pyridin-3- yl)benzyl)piperazin-1-yl)nicotinonitrile |
| 66 | 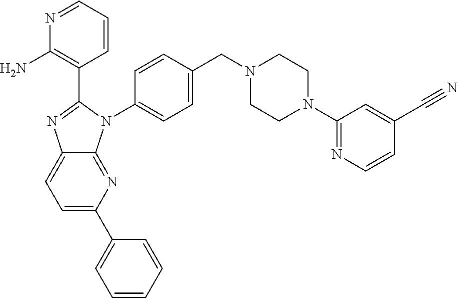 [SMILES: N#Cc1ccnc(N2CCN(Cc3ccc(-n4c(-c5cccnc5N)nc5ccc(-c6ccccc6)nc54)cc3)CC2)c1] | 2-(4-(4-(2-(2-aminopyridin-3-yl)-5-phenyl- 3H-imidazo[4,5-b]pyridin-3- yl)benzyl)piperazin-1-yl)isonicotinonitrile |
| 67 | 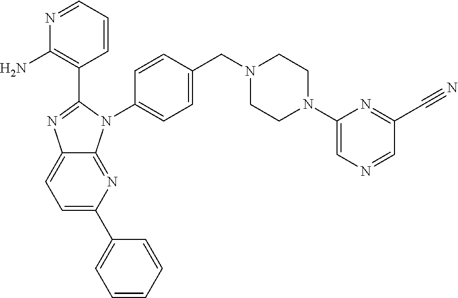 [SMILES: N#Cc1cncc(N2CCN(Cc3ccc(-n4c(-c5cccnc5N)nc5ccc(-c6ccccc6)nc54)cc3)CC2)n1] | 6-(4-(4-(2-(2-aminopyridin-3-yl)-5-phenyl- 3H-imidazo[4,5-b]pyridin-3- yl)benzyl)piperazin-1-yl)pyrazine-2- carbonitrile |
| 68 | 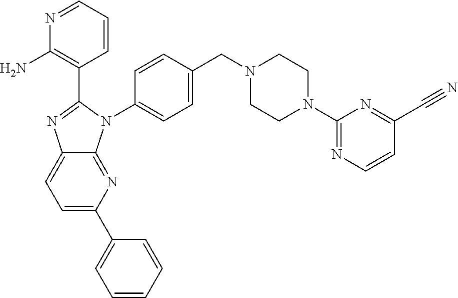 [SMILES: N#Cc1ccnc(N2CCN(Cc3ccc(-n4c(-c5cccnc5N)nc5ccc(-c6ccccc6)nc54)cc3)CC2)n1] | 2-(4-(4-(2-(2-aminopyridin-3-yl)-5-phenyl- 3H-imidazo[4,5-b ]pyridin-3- yl)benzyl)piperazin-1-yl)pyrimidine-4- carbonitrile |
| 69 | 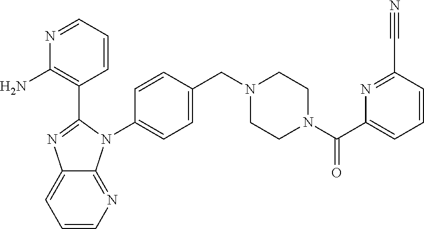 [SMILES: N#Cc1cccc(C(=O)N2CCN(Cc3ccc(-n4c(-c5cccnc5N)nc5cccnc54)cc3)CC2)n1] | 6-(4-(4-(2-(2-aminopyridin-3-yl)-3H- imidazo[4,5-b]pyridin-3- yl)benzyl)piperazine-1- carbonyl)picolinonitrile |
| 70 | 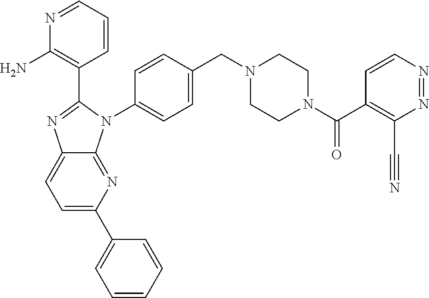 [SMILES: N#Cc1nnccc1C(=O)N1CCN(Cc2ccc(-n3c(-c4cccnc4N)nc4ccc(-c5ccccc5)nc43)cc2)CC1] | 4-(4-(4-(2-(2-aminopyridin-3-yl)-5-phenyl- 3H-imidazo[4,5-b ]pyridin-3- yl)benzyl)piperazine-1-carbonyl)pyridazine- 3-carbonitrile |
| 71 | 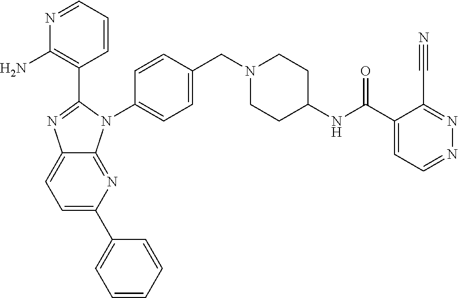 [SMILES: N#Cc1nnccc1C(=O)NC1CCN(Cc2ccc(-n3c(-c4cccnc4N)nc4ccc(-c5ccccc5)nc43)cc2)CC1] | N-(1-(4-(2-(2-aminopyridin-3-yl)-5-phenyl- 3H-imidazo[4,5-b]pyridin-3- yl)benzyl)piperidin-4-yl)-3- cyanopyridazine-4-carboxamide |
| 72 | 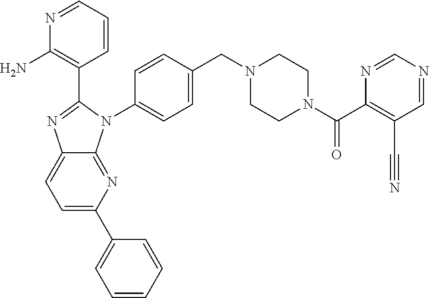 [SMILES: N#Cc1cncnc1C(=O)N1CCN(Cc2ccc(-n3c(-c4cccnc4N)nc4ccc(-c5ccccc5)nc43)cc2)CC1] | 4-(4-(4-(2-(2-aminopyridin-3-yl)-5-phenyl- 3H-imidazo[4,5-b]pyridin-3- yl)benzyl)piperazine-1- carbonyl)pyrimidine-5-carbonitrile |
| 73 | 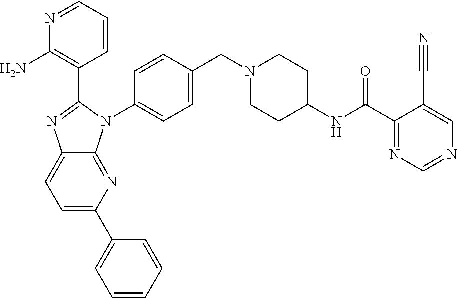 [SMILES: N#Cc1cncnc1C(=O)NC1CCN(Cc2ccc(-n3c(-c4cccnc4N)nc4ccc(-c5ccccc5)nc43)cc2)CC1] | N-(1-(4-(2-(2-aminopyridin-3-yl)-5-phenyl- 3H-imidazo[4,5-b]pyridin-3- yl)benzyl)piperidin-4-yl)-5- cyanopyrimidine-4-carboxamide |
| 74 | 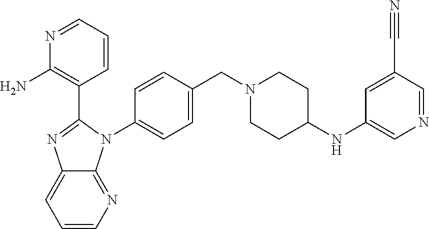 [SMILES: N#Cc1cncc(NC2CCN(Cc3ccc(-n4c(-c5cccnc5N)nc5cccnc54)cc3)CC2)c1] | 5-((1-(4-(2-(2-aminopyridin-3-yl)-3H- imidazo[4,5-b]pyridin-3- yl)benzyl)piperidin-4- yl)amino)nicotinonitrile |
| 75 | 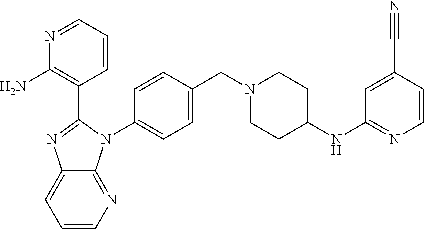 [SMILES: N#Cc1ccnc(NC2CCN(Cc3ccc(-n4c(-c5cccnc5N)nc5cccnc54)cc3)CC2)c1] | 2-((1-(4-(2-(2-aminopyridin-3-yl)-3H- imidazo[4,5-b]pyridin-3- yl)benzyl)piperidin-4- yl)amino)isonicotinonitrile |
| 76 | 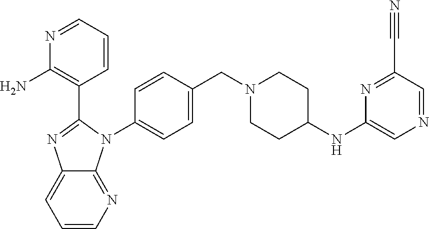 [SMILES: N#Cc1cncc(NC2CCN(Cc3ccc(-n4c(-c5cccnc5N)nc5cccnc54)cc3)CC2)n1] | 6-((1-(4-(2-(2-aminopyridin-3-yl)-3H- imidazo[4,5-b]pyridin-3- yl)benzyl)piperidin-4-yl)amino)pyrazine-2- carbonitrile |
| 77 | 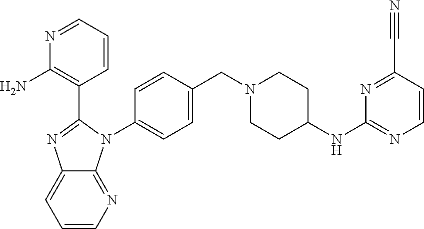 [SMILES: N#Cc1ccnc(NC2CCN(Cc3ccc(-n4c(-c5cccnc5N)nc5cccnc54)cc3)CC2)n1] | 2-((1-(4-(2-(2-aminopyridin-3-yl)-3H- imidazo[4,5-b]pyridin-3- yl)benzyl)piperidin-4-yl)amino)pyrimidine- 4-carbonitrile |
| 78 | 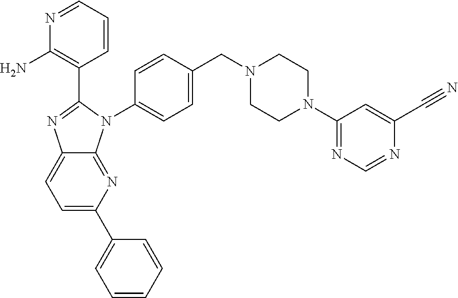 [SMILES: N#Cc1cc(N2CCN(Cc3ccc(-n4c(-c5cccnc5N)nc5ccc(-c6ccccc6)nc54)cc3)CC2)ncn1] | 6-(4-(4-(2-(2-aminopyridin-3-yl)-5-phenyl- 3H-imidazo[4,5-b ]pyridin-3- yl)benzyl)piperazin-1-yl)pyrimidine-4- carbonitrile |
| 79 | 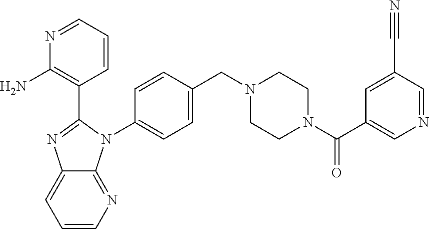 [SMILES: N#Cc1cncc(C(=O)N2CCN(Cc3ccc(-n4c(-c5cccnc5N)nc5cccnc54)cc3)CC2)c1] | 5-(4-(4-(2-(2-aminopyridin-3-yl)-3H- imidazo[4,5-b]pyridin-3- yl)benzyl)piperazine-1- carbonyl)nicotinonitrile |
| 80 | 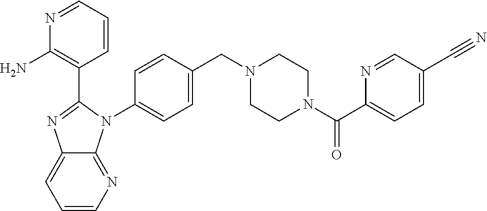 [SMILES: N#Cc1ccc(C(=O)N2CCN(Cc3ccc(-n4c(-c5cccnc5N)nc5cccnc54)cc3)CC2)nc1] | 6-(4-(4-(2-(2-aminopyridin-3-yl)-3H- imidazo[4,5-b]pyridin-3- yl)benzyl)piperazine-1- carbonyl)nicotinonitrile |
| 81 | 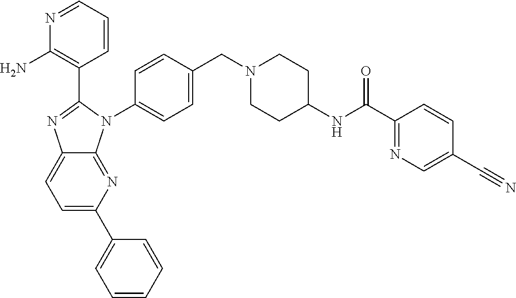 [SMILES: N#Cc1ccc(C(=O)NC2CCN(Cc3ccc(-n4c(-c5cccnc5N)nc5ccc(-c6ccccc6)nc54)cc3)CC2)nc1] | N-(1-(4-(2-(2-aminopyridin-3-yl)-5-phenyl- 3H-imidazo[4,5-b]pyridin-3- yl)benzyl)piperidin-4-yl)-5- cyanopicolinamide |
| 82 | 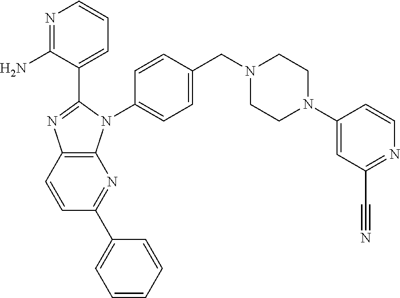 [SMILES: N#Cc1cc(N2CCN(Cc3ccc(-n4c(-c5cccnc5N)nc5ccc(-c6ccccc6)nc54)cc3)CC2)ccn1] | 4-(4-(4-(2-(2-aminopyridin-3-yl)-5-phenyl- 3H-imidazo[4,5-b ]pyridin-3- yl)benzyl)piperazin-1-yl)picolinonitrile |
| 83 | 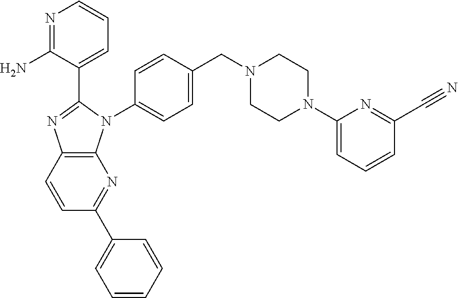 [SMILES: N#Cc1cccc(N2CCN(Cc3ccc(-n4c(-c5cccnc5N)nc5ccc(-c6ccccc6)nc54)cc3)CC2)n1] | 6-(4-(4-(2-(2-aminopyridin-3-yl)-5-phenyl- 3H-imidazo[4,5-b]pyridin-3- yl)benzyl)piperazin-1-yl)picolinonitrile |
| 84 | 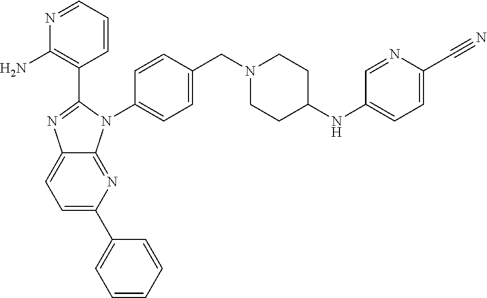 [SMILES: N#Cc1ccc(NC2CCN(Cc3ccc(-n4c(-c5cccnc5N)nc5ccc(-c6ccccc6)nc54)cc3)CC2)cn1] | 5-((1-(4-(2-(2-aminopyridin-3-yl)-5-phenyl- 3H-imidazo[4,5-b]pyridin-3- yl)benzyl)piperidin-4- yl)amino)picolinonitrile |
| 85 | 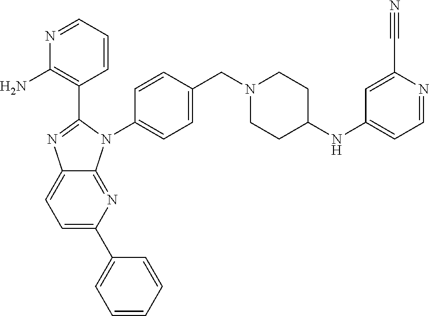 [SMILES: N#Cc1cc(NC2CCN(Cc3ccc(-n4c(-c5cccnc5N)nc5ccc(-c6ccccc6)nc54)cc3)CC2)ccn1] | 4-((1-(4-(2-(2-aminopyridin-3-yl)-5-phenyl- 3H-imidazo[4,5-b]pyridin-3- yl)benzyl)piperidin-4- yl)amino)picolinonitrile |
| 86 | 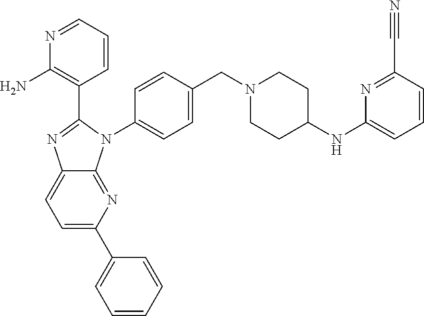 [SMILES: N#Cc1cccc(NC2CCN(Cc3ccc(-n4c(-c5cccnc5N)nc5ccc(-c6ccccc6)nc54)cc3)CC2)n1] | 6-((1-(4-(2-(2-aminopyridin-3-yl)-5-phenyl- 3H-imidazo[4,5-b]pyridin- 3- yl)benzyl)piperidin-4- yl)amino)picolinonitrile |
| 87 | 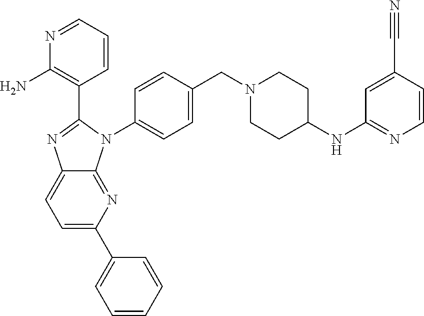 [SMILES: N#Cc1ccnc(NC2CCN(Cc3ccc(-n4c(-c5cccnc5N)nc5ccc(-c6ccccc6)nc54)cc3)CC2)c1] | 2-((1-(4-(2-(2-aminopyridin-3-yl)-5-phenyl- 3H-imidazo[4,5-b]pyridin-3- yl)benzyl)piperidin-4- yl)amino)isonicotinonitrile |
| 88 | 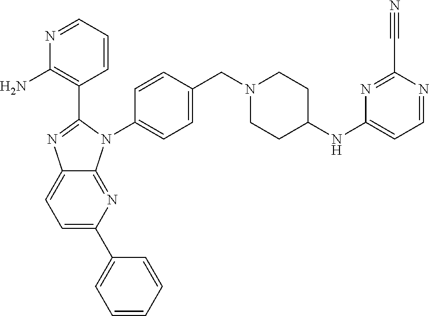 [SMILES: N#Cc1nccc(NC2CCN(Cc3ccc(-n4c(-c5cccnc5N)nc5ccc(-c6ccccc6)nc54)cc3)CC2)n1] | 4-((1- (4-(2-(2-aminopyridin-3-yl)-5-phenyl- 3H-imidazo[4,5-b]pyridin-3- yl)benzyl)piperidin-4-yl)amino)pyrimidine- 2-carbonitrile |
| 89 | 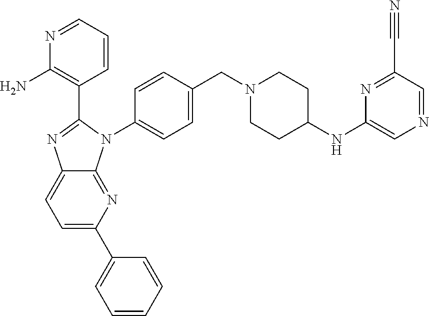 [SMILES: N#Cc1cncc(NC2CCN(Cc3ccc(-n4c(-c5cccnc5N)nc5ccc(-c6ccccc6)nc54)cc3)CC2)n1] | 6-((1-(4-(2-(2-aminopyridin-3-yl)-5-phenyl- 3H-imidazo[4,5-b]pyridin-3- yl)benzyl)piperidin-4-yl)amino)pyrazine-2- carbonitrile |
| 90 | 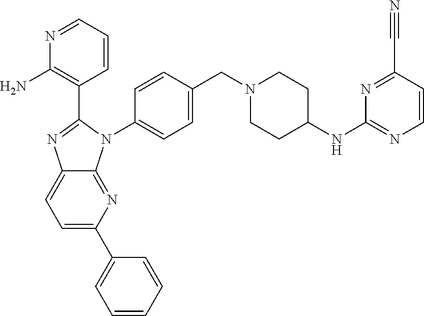 [SMILES: N#Cc1ccnc(NC2CCN(Cc3ccc(-n4c(-c5cccnc5N)nc5ccc(-c6ccccc6)nc54)cc3)CC2)n1] | 2-((1-(4-(2-(2-aminopyridin-3-yl)-5-phenyl- 3H-imidazo[4,5-b]pyridin-3- yl)benzyl)piperidin-4-yl)amino)pyrimidine- 4-carbonitrile |
| 91 | 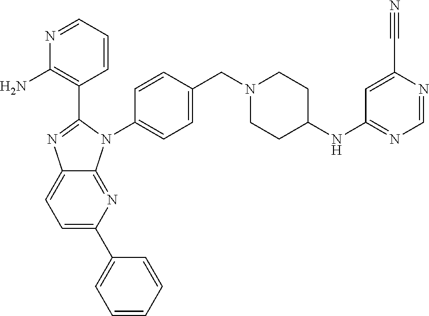 [SMILES: N#Cc1cc(NC2CCN(Cc3ccc(-n4c(-c5cccnc5N)nc5ccc(-c6ccccc6)nc54)cc3)CC2)ncn1] | 6-((1-(4-(2-(2-aminopyridin-3-yl)-5-phenyl- 3H-imidazo[4,5-b]pyridin-3- yl)benzyl)piperidin-4-yl)amino)pyrimidine- 4-carbonitrile |
| 92 | 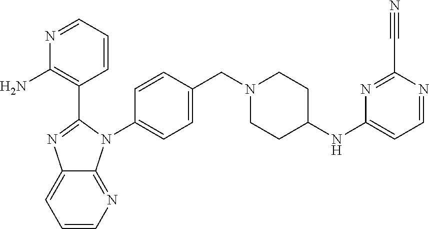 [SMILES: N#Cc1nccc(NC2CCN(Cc3ccc(-n4c(-c5cccnc5N)nc5cccnc54)cc3)CC2)n1] | 4-((1-(4-(2-(2-aminopyridin-3-yl)-3H- imidazo[4,5-b]pyridin-3- yl)benzyl)piperidin-4-yl)amino)pyrimidine- 2-carbonitrile |
| 93 | 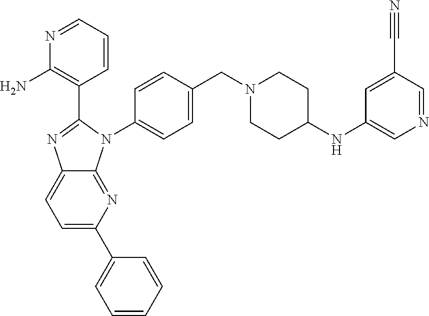 [SMILES: N#Cc1cncc(NC2CCN(Cc3ccc(-n4c(-c5cccnc5N)nc5ccc(-c6ccccc6)nc54)cc3)CC2)c1] | 5-((1-(4-(2-(2-aminopyridin-3-yl)-5-phenyl- 3H-imidazo[4,5-b]pyridin-3- yl)benzyl)piperidin-4- yl)amino)nicotinonitrile |
| 94 | 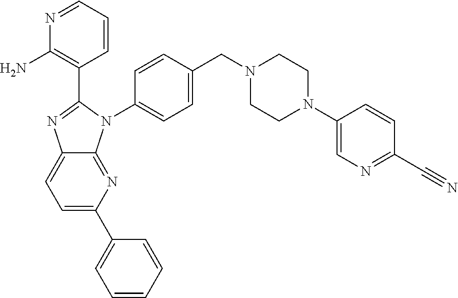 [SMILES: N#Cc1ccc(N2CCN(Cc3ccc(-n4c(-c5cccnc5N)nc5ccc(-c6ccccc6)nc54)cc3)CC2)cn1] | 5-(4-(4-(2-(2-aminopyridin-3-yl)-5-phenyl- 3H-imidazo[4,5-b]pyridin-3- yl)benzyl)piperazin-1-yl)picolinonitrile |
| 95 | 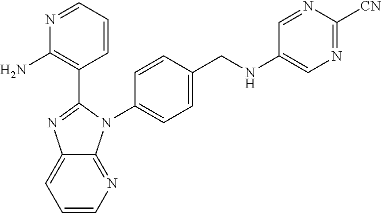 [SMILES: N#Cc1ncc(NCc2ccc(-n3c(-c4cccnc4N)nc4cccnc43)cc2)cn1] | 5-((4-(2-(2-aminopyridin-3-yl)-3H- imidazo[4,5-b]pyridin-3- yl)benzyl)amino)pyrimidine-2-carbonitrile |
| 96 | 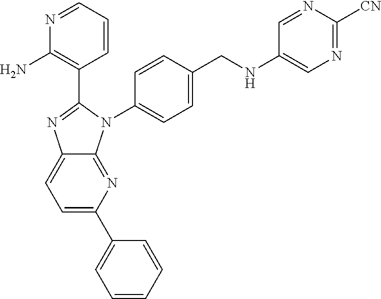 [SMILES: N#Cc1ncc(NCc2ccc(-n3c(-c4cccnc4N)nc4ccc(-c5ccccc5)nc43)cc2)cn1] | 5-((4-(2-(2-aminopyridin-3-yl)-5-phenyl- 3H-imidazo[4,5-b]pyridin-3- yl)benzyl)amino)pyrimidine-2-carbonitrile |
| 97 | 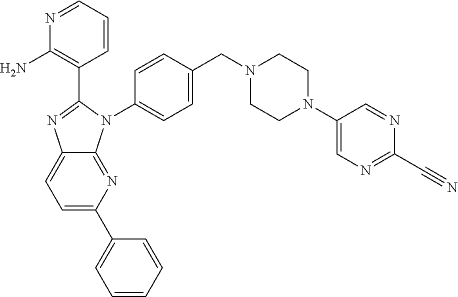 [SMILES: N#Cc1ncc(N2CCN(Cc3ccc(-n4c(-c5cccnc5N)nc5ccc(-c6ccccc6)nc54)cc3)CC2)cn1] | 5- (4-(4-(2-(2-aminopyridin-3-yl)-5-phenyl- 3H-imidazo[4,5-b]pyridin-3- yl)benzyl)piperazin-1-yl)pyrimidine-2- carbonitrile |
| 98 | 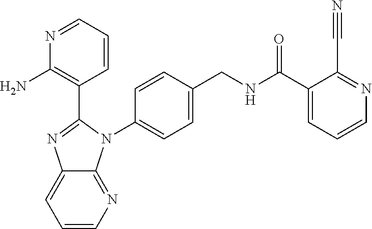 [SMILES: N#Cc1ncccc1C(=O)NCc1ccc(-n2c(-c3cccnc3N)nc3cccnc32)cc1] | N-(4-(2-(2-aminopyridin-3-yl)-3H- imidazo[4,5-b]pyridin-3-yl)benzyl)-2- cyanonicotinamide |
| 99 | 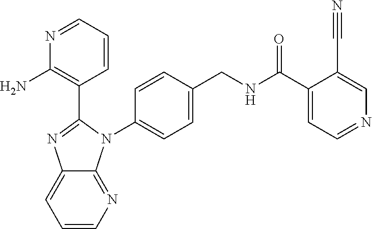 [SMILES: N#Cc1cnccc1C(=O)NCc1ccc(-n2c(-c3cccnc3N)nc3cccnc32)cc1] | N-(4-(2-(2-aminopyridin-3-yl)-3H- imidazo[4,5-b]pyridin-3-yl)benzyl)-3- cyanoisonicotinamide |
| 100 | 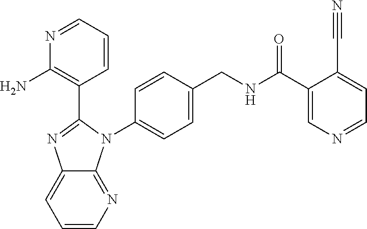 [SMILES: N#Cc1ccncc1C(=O)NCc1ccc(-n2c(-c3cccnc3N)nc3cccnc32)cc1] | N-(4-(2-(2-aminopyridin-3-yl)-3H- imidazo[4,5-b]pyridin-3-yl)benzyl)-4- cyanonicotinamide |
| 101 | 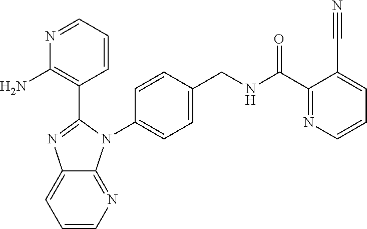 [SMILES: N#Cc1cccnc1C(=O)NCc1ccc(-n2c(-c3cccnc3N)nc3cccnc32)cc1] | N-(4-(2-(2-aminopyridin-3-yl)-3H- imidazo[4,5-b]pyridin-3-yl)benzyl)-3- cyanopicolinamide |
| 102 | 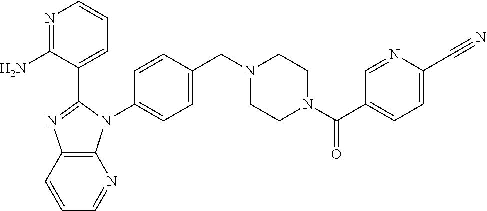 [SMILES: N#Cc1ccc(C(=O)N2CCN(Cc3ccc(-n4c(-c5cccnc5N)nc5cccnc54)cc3)CC2)cn1] | 5-(4-(4-(2-(2-aminopyridin-3-yl)-3H- imidazo[4,5-b]pyridin-3- yl)benzyl)piperazine-1- carbonyl)picolinonitrile |
| 103 | 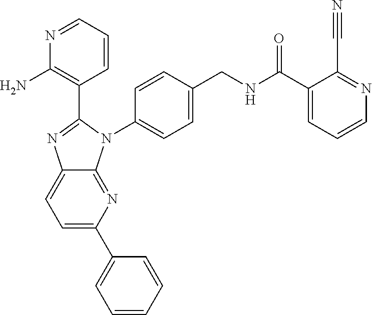 [SMILES: N#Cc1ncccc1C(=O)NCc1ccc(-n2c(-c3cccnc3N)nc3ccc(-c4ccccc4)nc32)cc1] | N-(4-(2-(2-aminopyridin-3-yl)-5-phenyl- 3H-imidazo[4,5-b]pyridin-3-yl)benzyl)-2- cyanonicotinamide |
| 104 | 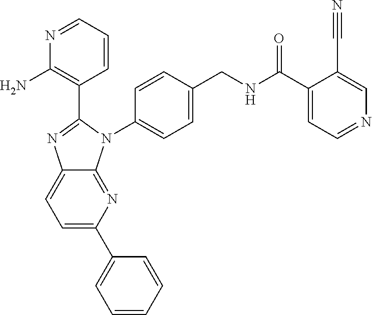 [SMILES: N#Cc1cnccc1C(=O)NCc1ccc(-n2c(-c3cccnc3N)nc3ccc(-c4ccccc4)nc32)cc1] | N-(4-(2-(2-aminopyridin-3-yl)-5-phenyl- 3H-imidazo[4,5-b]pyridin-3-yl)benzyl)-3- cyanoisonicotinamide |
| 105 | 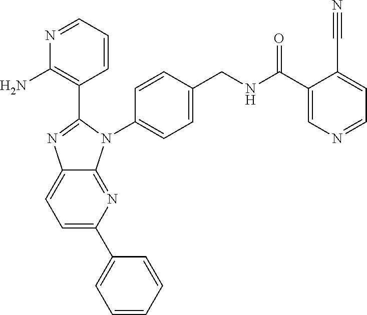 [SMILES: N#Cc1ccncc1C(=O)NCc1ccc(-n2c(-c3cccnc3N)nc3ccc(-c4ccccc4)nc32)cc1] | N-(4-(2-(2-aminopyridin-3-yl)-5-phenyl- 3H-imidazo[4,5-b]pyridin-3-yl)benzyl)-4- cyanonicotinamide |
| 106 | 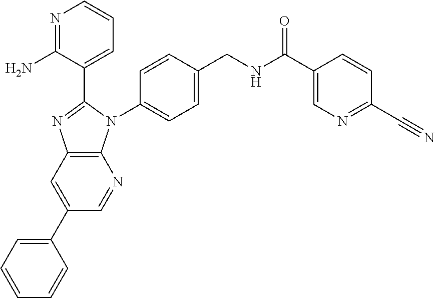 [SMILES: N#Cc1ccc(C(=O)NCc2ccc(-n3c(-c4cccnc4N)nc4cc(-c5ccccc5)cnc43)cc2)cn1] | N-(4-(2-(2-aminopyridin-3-yl)-6-phenyl- 3H-imidazo[4,5-b]pyridin-3-yl)benzyl)-6- cyanonicotinamide |
| 107 | 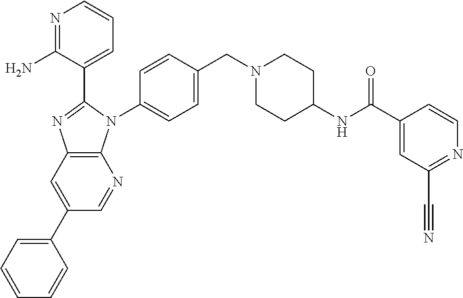 [SMILES: N#Cc1cc(C(=O)NC2CCN(Cc3ccc(-n4c(-c5cccnc5N)nc5cc(-c6ccccc6)cnc54)cc3)CC2)ccn1] | N-(1-(4-(2-(2-aminopyridin-3-yl)-6-phenyl- 3H-imidazo[4,5-b]pyridin-3- yl)benzyl)piperidin-4-yl)-2- cyanoisonicotinamide |
| 108 | 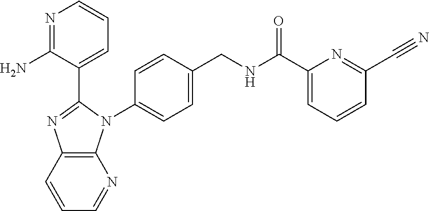 [SMILES: N#Cc1cccc(C(=O)NCc2ccc(-n3c(-c4cccnc4N)nc4cccnc43)cc2)n1] | N-(4-(2-(2-aminopyridin-3-yl)-3H- imidazo[4,5-b]pyridin-3-yl)benzyl)-6- cyanopicolinamide |
| 109 | 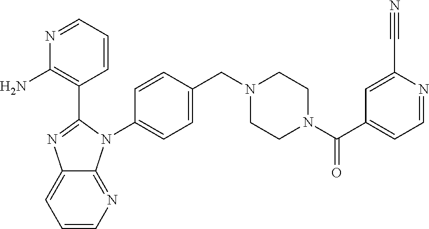 [SMILES: N#Cc1cc(C(=O)N2CCN(Cc3ccc(-n4c(-c5cccnc5N)nc5cccnc54)cc3)CC2)ccn1] | 4-(4-(4-(2-(2-aminopyridin-3-yl)-3H- imidazo[4,5-b]pyridin-3- yl)benzyl)piperazine-1- carbonyl)picolinonitrile |
| 110 | 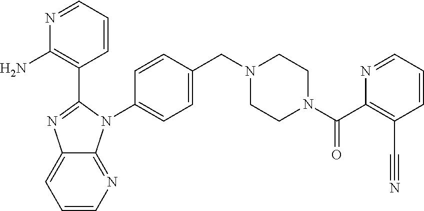 [SMILES: N#Cc1cccnc1C(=O)N1CCN(Cc2ccc(-n3c(-c4cccnc4N)nc4cccnc43)cc2)CC1] | 2-(4-(4-(2-(2-aminopyridin-3-yl)-3H- imidazo[4,5-b]pyridin-3- yl)benzyl)piperazine-1- carbonyl)nicotinonitrile |
| 111 | 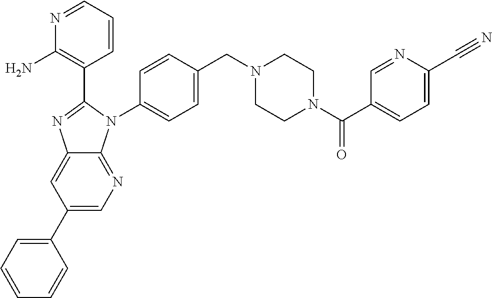 [SMILES: N#Cc1ccc(C(=O)N2CCN(Cc3ccc(-n4c(-c5cccnc5N)nc5cc(-c6ccccc6)cnc54)cc3)CC2)cn1] | 5-(4-(4-(2-(2-aminopyridin-3-yl)-6-phenyl- 3H-imidazo[4,5-b]pyridin-3- yl)benzyl)piperazine-1- carbonyl)picolinonitrile |
| 112 | 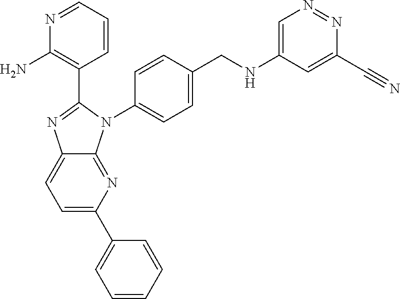 [SMILES: N#Cc1cc(NCc2ccc(-n3c(-c4cccnc4N)nc4ccc(-c5ccccc5)nc43)cc2)cnn1] | 5-((4-(2-(2-aminopyridin-3-yl)-5-phenyl- 3H-imidazo[4,5-b]pyridin-3- yl)benzyl)amino)pyridazine-3-carbonitrile |
| 113 | 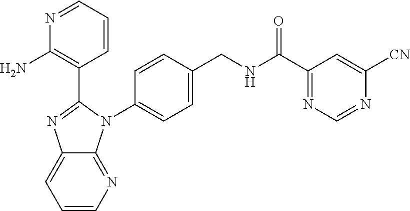 [SMILES: N#Cc1cc(C(=O)NCc2ccc(-n3c(-c4cccnc4N)nc4cccnc43)cc2)ncn1] | N-(4-(2-(2-aminopyridin-3-yl)-3H- imidazo[4,5-b]pyridin-3-yl)benzyl)-6- cyanopyrimidine-4-carboxamide |
| 114 | 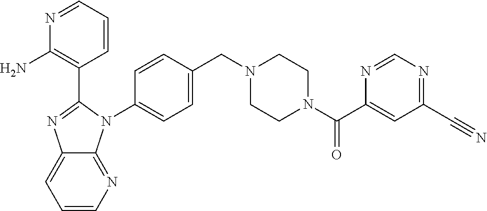 [SMILES: N#Cc1cc(C(=O)N2CCN(Cc3ccc(-n4c(-c5cccnc5N)nc5cccnc54)cc3)CC2)ncn1] | 6-(4-(4-(2-(2-aminopyridin-3-yl)-3H- imidazo[4,5-b]pyridin-3- yl)benzyl)piperazine-1- carbonyl)pyrimidine-4-carbonitrile |
| 115 | 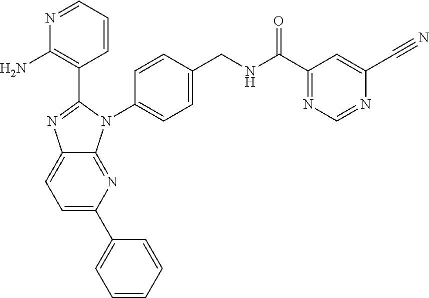 [SMILES: N#Cc1cc(C(=O)NCc2ccc(-n3c(-c4cccnc4N)nc4ccc(-c5ccccc5)nc43)cc2)ncn1] | N-(4-(2-(2-aminopyridin-3-yl)-5-phenyl- 3H-imidazo[4,5-b]pyridin-3-yl)benzyl)-6- cyanopyrimidine-4-carboxamide |
| 116 | 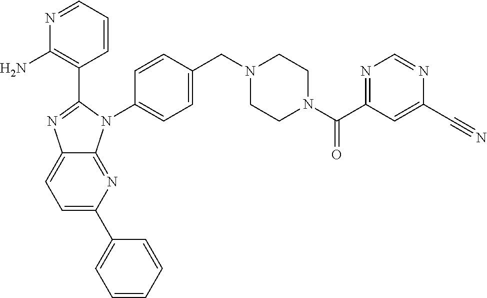 [SMILES: N#Cc1cc(C(=O)N2CCN(Cc3ccc(-n4c(-c5cccnc5N)nc5ccc(-c6ccccc6)nc54)cc3)CC2)ncn1] | 6-(4- (4-(2-(2-aminopyridin-3-yl)-5-phenyl- 3H-imidazo[4,5-b]pyridin-3- yl)benzyl)piperazine-1- carbonyl)pyrimidine-4-carbonitrile |
| 117 | 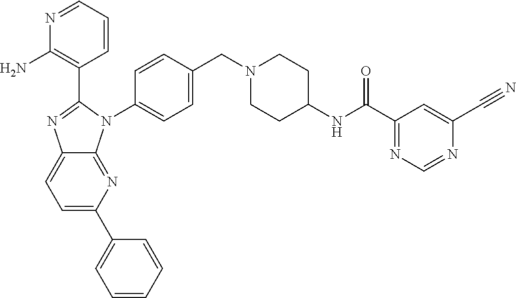 [SMILES: N#Cc1cc(C(=O)NC2CCN(Cc3ccc(-n4c(-c5cccnc5N)nc5ccc(-c6ccccc6)nc54)cc3)CC2)ncn1] | N-(1-(4-(2-(2-aminopyridin-3-yl)-5-phenyl- 3H-imidazo[4,5-b]pyridin-3- yl)benzyl)piperidin-4-yl)-6- cyanopyrimidine-4-carboxamide |
| 118 | 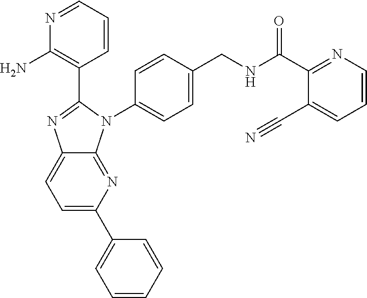 [SMILES: N#Cc1cccnc1C(=O)NCc1ccc(-n2c(-c3cccnc3N)nc3ccc(-c4ccccc4)nc32)cc1] | N-(4-(2-(2-aminopyridin-3-yl)-5-phenyl- 3H-imidazo[4,5-b]pyridin-3-yl)benzyl)-3- cyanopicolinamide |
| 119 | 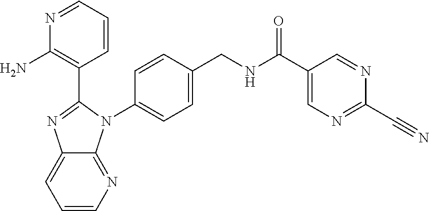 [SMILES: N#Cc1ncc(C(=O)NCc2ccc(-n3c(-c4cccnc4N)nc4cccnc43)cc2)cn1] | N-(4-(2-(2-aminopyridin-3-yl)-3H- imidazo[4,5-b]pyridin-3-yl)benzyl)-2- cyanopyrimidine-5-carboxamide |
| 120 | 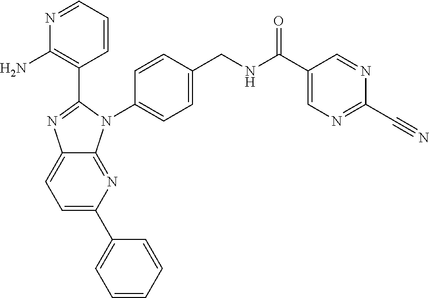 [SMILES: N#Cc1ncc(C(=O)NCc2ccc(-n3c(-c4cccnc4N)nc4ccc(-c5ccccc5)nc43)cc2)cn1] | N-(4-(2-(2-aminopyridin-3-yl)-5-phenyl- 3H-imidazo[4,5-b]pyridin-3-yl)benzyl)-2- cyanopyrimidine-5-carboxamide |
| 121 | 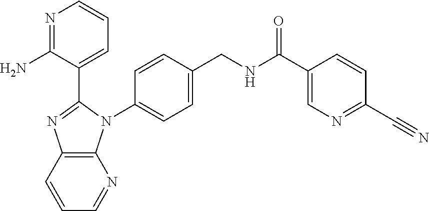 [SMILES: N#Cc1ccc(C(=O)NCc2ccc(-n3c(-c4cccnc4N)nc4cccnc43)cc2)cn1] | N-(4-(2-(2-aminopyridin-3-yl)-3H- imidazo[4,5-b]pyridin-3-yl)benzyl)-6- cyanonicotinamide |
| 122 | 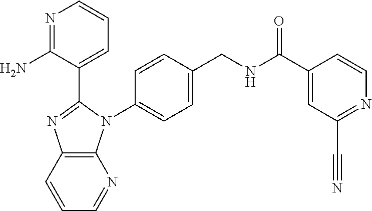 [SMILES: N#Cc1cc(C(=O)NCc2ccc(-n3c(-c4cccnc4N)nc4cccnc43)cc2)ccn1] | N-(4-(2-(2-aminopyridin-3-yl)-3H- imidazo[4,5-b]pyridin-3-yl)benzyl)-2- cyanoisonicotinamide |
| 123 | 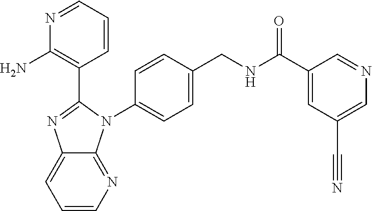 [SMILES: N#Cc1cncc(C(=O)NCc2ccc(-n3c(-c4cccnc4N)nc4cccnc43)cc2)c1] | N-(4-(2-(2-aminopyridin-3-yl)-3H- imidazo[4,5-b]pyridin-3-yl)benzyl)-5- cyanonicotinamide |
| 124 | 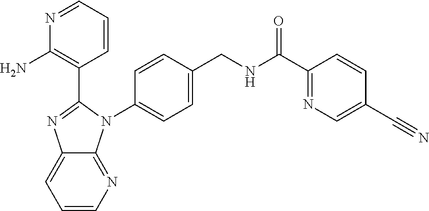 [SMILES: N#Cc1ccc(C(=O)NCc2ccc(-n3c(-c4cccnc4N)nc4cccnc43)cc2)nc1] | N-(4-(2-(2-aminopyridin-3-yl)-3H- imidazo[4,5-b]pyridin-3-yl)benzyl)-5- cyanopicolinamide |
| 125 | 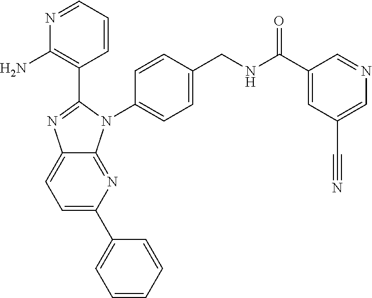 [SMILES: N#Cc1cncc(C(=O)NCc2ccc(-n3c(-c4cccnc4N)nc4ccc(-c5ccccc5)nc43)cc2)c1] | N-(4-(2-(2-aminopyridin-3-yl)-5-phenyl- 3H-imidazo[4,5-b]pyridin-3-yl)benzyl)-5- cyanonicotinamide |
| 126 | 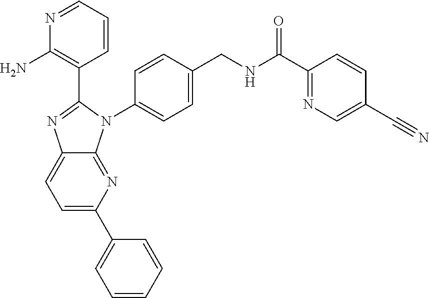 [SMILES: N#Cc1ccc(C(=O)NCc2ccc(-n3c(-c4cccnc4N)nc4ccc(-c5ccccc5)nc43)cc2)nc1] | N-(4-(2-(2-aminopyridin-3-yl)-5-phenyl- 3H-imidazo[4,5-b]pyridin-3-yl)benzyl)-5- cyanopicolinamide |
| 127 | 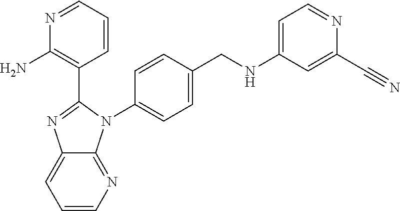 [SMILES: N#Cc1cc(NCc2ccc(-n3c(-c4cccnc4N)nc4cccnc43)cc2)ccn1] | 4-((4-(2-(2-aminopyridin-3-yl)-3H- imidazo[4,5-b]pyridin-3- yl)benzyl)amino)picolinonitrile |
| 128 | 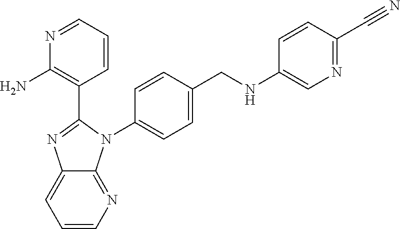 [SMILES: N#Cc1ccc(NCc2ccc(-n3c(-c4cccnc4N)nc4cccnc43)cc2)cn1] | 5-((4-(2-(2-aminopyridin-3-yl)-3H- imidazo[4,5-b]pyridin-3- yl)benzyl)amino)picolinonitrile |
| 129 | 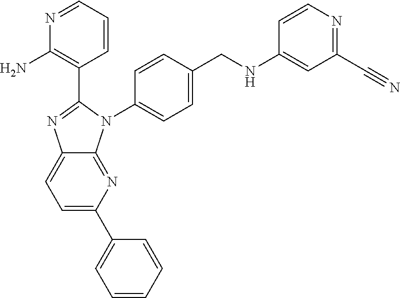 [SMILES: N#Cc1cc(NCc2ccc(-n3c(-c4cccnc4N)nc4ccc(-c5ccccc5)nc43)cc2)ccn1] | 4-((4-(2-(2-aminopyridin-3-yl)-5-phenyl- 3H-imidazo[4,5-b]pyridin-3- yl)benzyl)amino)picolinonitrile |
| 130 | 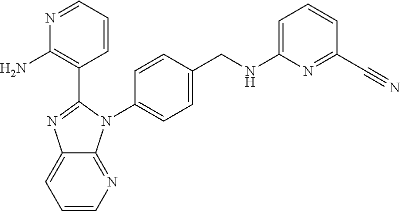 [SMILES: N#Cc1cccc(NCc2ccc(-n3c(-c4cccnc4N)nc4cccnc43)cc2)n1] | 6-((4-(2-(2-aminopyridin-3-yl)-3H- imidazo[4,5-b]pyridin-3- yl)benzyl)amino)picolinonitrile |
| 131 | 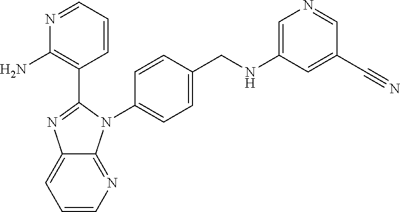 [SMILES: N#Cc1cncc(NCc2ccc(-n3c(-c4cccnc4N)nc4cccnc43)cc2)c1] | 5-((4-(2-(2-aminopyridin-3-yl)-3H- imidazo[4,5-b]pyridin-3- yl)benzyl)amino)nicotinonitrile |
| 132 | 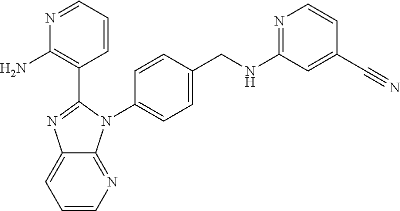 [SMILES: N#Cc1ccnc(NCc2ccc(-n3c(-c4cccnc4N)nc4cccnc43)cc2)c1] | 2-((4-(2-(2-aminopyridin-3-yl)-3H- imidazo[4,5-b]pyridin-3- yl)benzyl)amino)isonicotinonitrile |
| 133 | 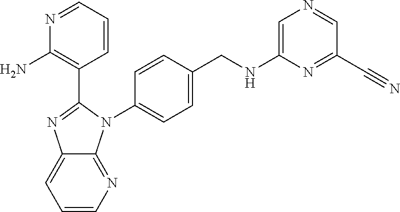 [SMILES: N#Cc1cncc(NCc2ccc(-n3c(-c4cccnc4N)nc4cccnc43)cc2)n1] | 6-((4-(2-(2-aminopyridin-3-yl)-3H- imidazo[4,5-b]pyridin-3- yl)benzyl)amino)pyrazine-2-carbonitrile |
| 134 | 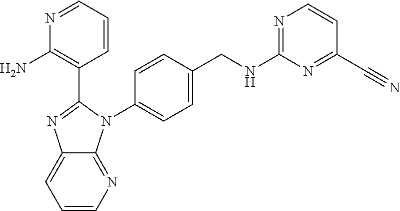 [SMILES: N#Cc1ccnc(NCc2ccc(-n3c(-c4cccnc4N)nc4cccnc43)cc2)n1] | 2-((4-(2-(2-aminopyridin-3-yl)-3H- imidazo[4,5-b]pyridin-3- yl)benzyl)amino)pyrimidine-4-carbonitrile |
| 135 | 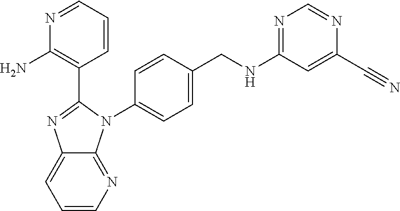 [SMILES: N#Cc1cc(NCc2ccc(-n3c(-c4cccnc4N)nc4cccnc43)cc2)ncn1] | 6-((4-(2-(2-aminopyridin-3-yl)-3H- imidazo[4,5-b]pyridin-3- yl)benzyl)amino)pyrimidine-4-carbonitrile |
| 136 | 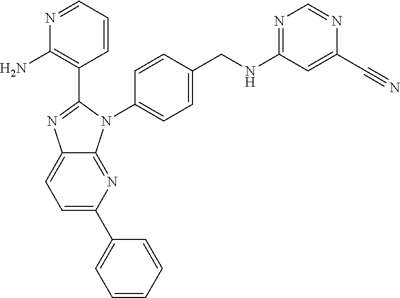 [SMILES: N#Cc1cc(NCc2ccc(-n3c(-c4cccnc4N)nc4ccc(-c5ccccc5)nc43)cc2)ncn1] | 6-((4-(2-(2-aminopyridin-3-yl)-5-phenyl- 3H-imidazo[4,5-b]pyridin-3- yl)benzyl)amino)pyrimidine-4-carbonitrile |
| 137 | 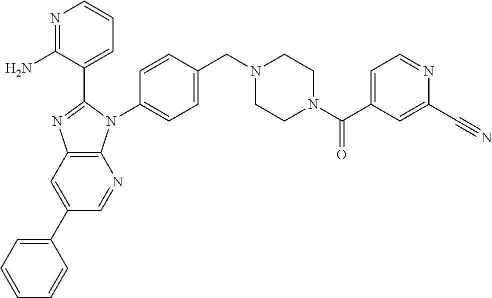 [SMILES: N#Cc1cc(C(=O)N2CCN(Cc3ccc(-n4c(-c5cccnc5N)nc5cc(-c6ccccc6)cnc54)cc3)CC2)ccn1] | 4-(4-(4-(2-(2-aminopyridin-3-yl)-6-phenyl- 3H-imidazo[4,5-b]pyridin-3- yl)benzyl)piperazine-1- carbonyl)picolinonitrile |
| 138 | 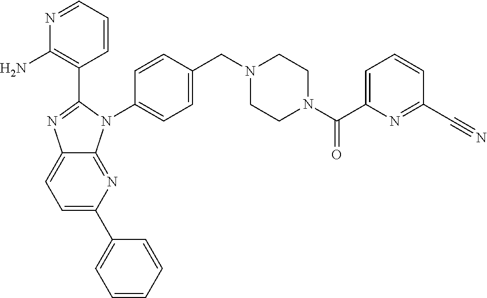 [SMILES: N#Cc1cccc(C(=O)N2CCN(Cc3ccc(-n4c(-c5cccnc5N)nc5ccc(-c6ccccc6)nc54)cc3)CC2)n1] | 6-(4-(4-(2-(2-aminopyridin-3-yl)-5-phenyl- 3H-imidazo[4,5-b]pyridin-3- yl)benzyl)piperazine-1- carbonyl)picolinonitrile |
| 139 | 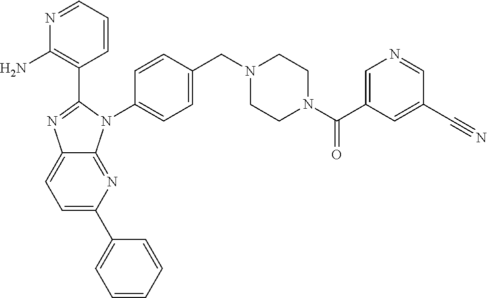 [SMILES: N#Cc1cncc(C(=O)N2CCN(Cc3ccc(-n4c(-c5cccnc5N)nc5ccc(-c6ccccc6)nc54)cc3)CC2)c1] | S-(4-(4-(2-(2-aminopyridin-3-yl)-5-phenyl- 3H-imidazo[4,5-b ]pyridin-3- yl)benzyl)piperazine-1- carbonyl)nicotinonitrile |
| 140 | 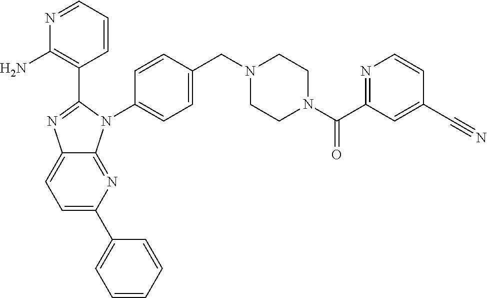 [SMILES: N#Cc1ccnc(C(=O)N2CCN(Cc3ccc(-n4c(-c5cccnc5N)nc5ccc(-c6ccccc6)nc54)cc3)CC2)c1] | 2-(4-(4-(2-(2-aminopyridin-3-yl)-5-phenyl- 3H-imidazo[4,5-b]pyridin-3- yl)benzyl)piperazine-1- carbonyl)isonicotinonitrile |
| 141 | 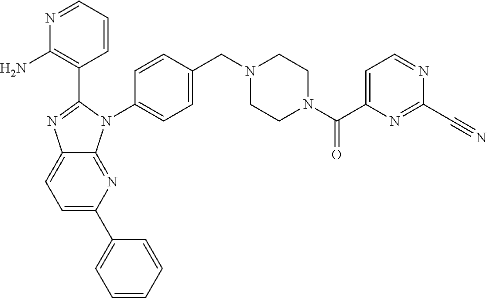 [SMILES: N#Cc1nccc(C(=O)N2CCN(Cc3ccc(-n4c(-c5cccnc5N)nc5ccc(-c6ccccc6)nc54)cc3)CC2)n1] | 4-(4-(4-(2-(2-aminopyridin-3-yl)-5-phenyl- 3H-imidazo[4,5-b]pyridin-3- yl)benzyl)piperazine-1- carbonyl)pyrimidine-2-carbonitrile |
| 142 | 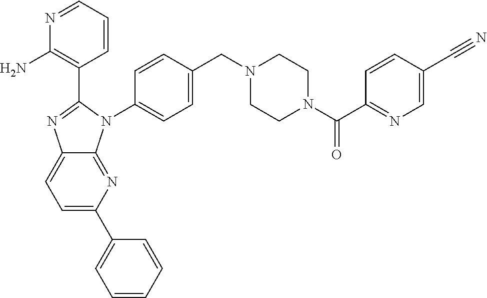 [SMILES: N#Cc1ccc(C(=O)N2CCN(Cc3ccc(-n4c(-c5cccnc5N)nc5ccc(-c6ccccc6)nc54)cc3)CC2)nc1] | 6-(4-(4-(2-(2-aminopyridin-3-yl)-5-phenyl- 3H-imidazo[4,5-b]pyridin-3- yl)benzyl)piperazine-1- carbonyl)nicotinonitrile |
| 143 | 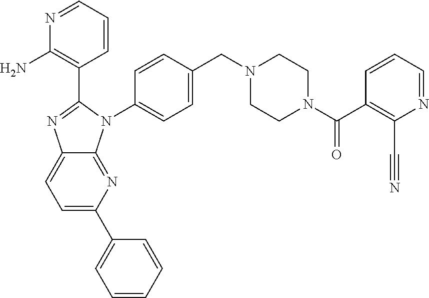 [SMILES: N#Cc1ncccc1C(=O)N1CCN(Cc2ccc(-n3c(-c4cccnc4N)nc4ccc(-c5ccccc5)nc43)cc2)CC1] | 3-(4-(4-(2-(2-aminopyridin-3-yl)-5-phenyl- 3H-imidazo[4,5-b]pyridin-3- yl)benzyl)piperazine-1- carbonyl)picolinonitrile |
| 144 | 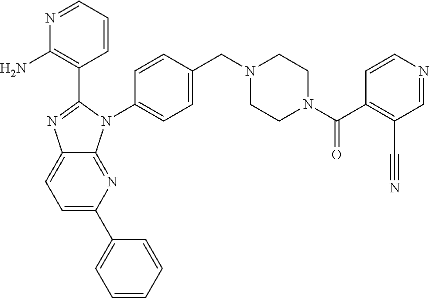 [SMILES: N#Cc1cnccc1C(=O)N1CCN(Cc2ccc(-n3c(-c4cccnc4N)nc4ccc(-c5ccccc5)nc43)cc2)CC1] | 4-(4-(4-(2-(2-aminopyridin-3-yl)-5-phenyl- 3H-imidazo[4,5-b]pyridin-3- yl)benzyl)piperazine-1- carbonyl)nicotinonitrile |
| 145 | 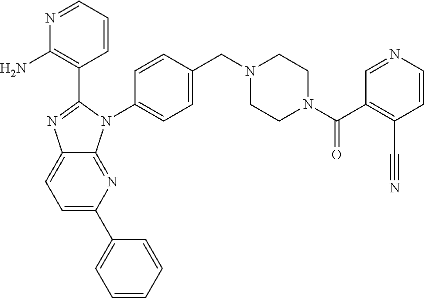 [SMILES: N#Cc1ccncc1C(=O)N1CCN(Cc2ccc(-n3c(-c4cccnc4N)nc4ccc(-c5ccccc5)nc43)cc2)CC1] | 3-(4-(4-(2-(2-aminopyridin-3-yl)-5-phenyl- 3H-imidazo[4,5-b]pyridin-3- yl)benzyl)piperazine-1- carbonyl)isonicotinonitrile |
| 146 | 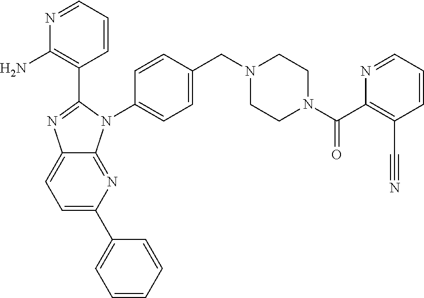 [SMILES: N#Cc1cccnc1C(=O)N1CCN(Cc2ccc(-n3c(-c4cccnc4N)nc4ccc(-c5ccccc5)nc43)cc2)CC1] | 2-(4-(4-(2-(2-aminopyridin-3-yl)-5-phenyl- 3H-imidazo[4,5-b ]pyridin-3- yl)benzyl)piperazine-1- carbonyl)nicotinonitrile |
| 147 | 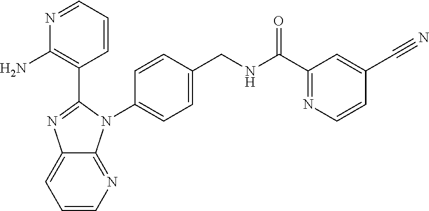 [SMILES: N#Cc1ccnc(C(=O)NCc2ccc(-n3c(-c4cccnc4N)nc4cccnc43)cc2)c1] | N-(4-(2-(2-aminopyridin-3-yl)-3H- imidazo[4,5-b]pyridin-3-yl)benzyl)-4- cyanopicolinamide |
| 148 | 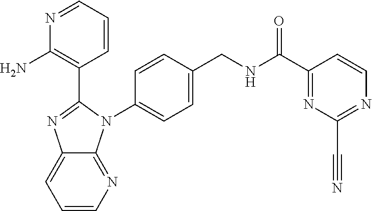 [SMILES: N#Cc1nccc(C(=O)NCc2ccc(-n3c(-c4cccnc4N)nc4cccnc43)cc2)n1] | N-(4-(2-(2-aminopyridin-3-yl)-3H- imidazo[4,5-b]pyridin-3-yl)benzyl)-2- cyanopyrimidine-4-carboxamide |
| 149 | 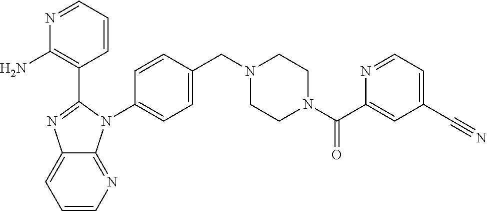 [SMILES: N#Cc1ccnc(C(=O)N2CCN(Cc3ccc(-n4c(-c5cccnc5N)nc5cccnc54)cc3)CC2)c1] | 2-(4-(4-(2-(2-aminopyridin-3-yl)-3H- imidazo[4,5-b]pyridin-3- yl)benzyl)piperazine-1- carbonyl)isonicotinonitrile |
| 150 | 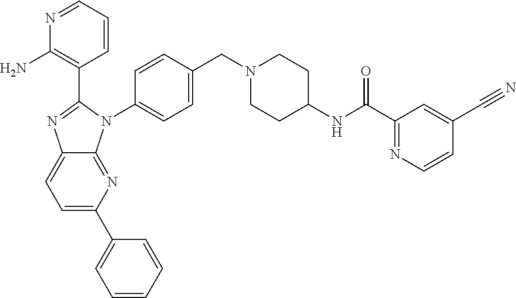 [SMILES: N#Cc1ccnc(C(=O)NC2CCN(Cc3ccc(-n4c(-c5cccnc5N)nc5ccc(-c6ccccc6)nc54)cc3)CC2)c1] | N-(1-(4-(2-(2-aminopyridin-3-yl)-5-phenyl- 3H-imidazo[4,5-b]pyridin-3- yl)benzyl)piperidin-4-yl)-4- cyanopicolinamide |
| 151 | 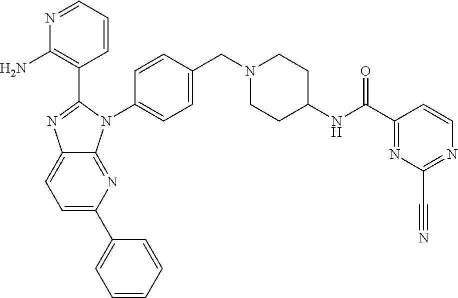 [SMILES: N#Cc1nccc(C(=O)NC2CCN(Cc3ccc(-n4c(-c5cccnc5N)nc5ccc(-c6ccccc6)nc54)cc3)CC2)n1] | N-(1-(4-(2-(2-aminopyridin-3-yl)-5-phenyl- 3H-imidazo[4,5-b]pyridin-3- yl)benzyl)piperidin-4-yl)-2- cyanopyrimidine-4-carboxamide |
| 152 | 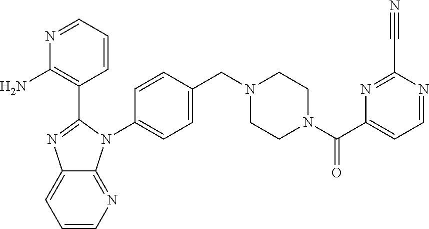 [SMILES: N#Cc1nccc(C(=O)N2CCN(Cc3ccc(-n4c(-c5cccnc5N)nc5cccnc54)cc3)CC2)n1] | 4-(4-(4-(2-(2-aminopyridin-3-yl)-3H- imidazo[4,5-b]pyridin-3- yl)benzyl)piperazine-1- carbonyl)pyrimidine-2-carbonitrile |
| 153 | 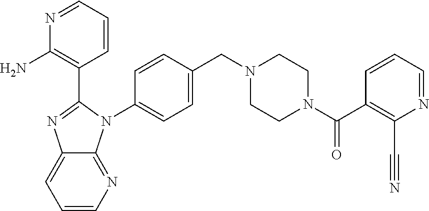 [SMILES: N#Cc1ncccc1C(=O)N1CCN(Cc2ccc(-n3c(-c4cccnc4N)nc4cccnc43)cc2)CC1] | 3-(4-(4-(2-(2-aminopyridin-3-yl)-3H- imidazo[4,5-b]pyridin-3- yl)benzyl)piperazine-1- carbonyl)picolinonitrile |
| 154 | 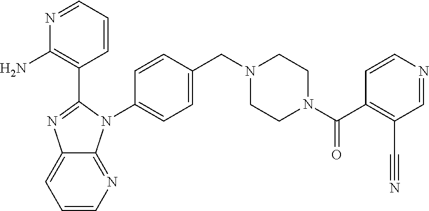 [SMILES: N#Cc1cnccc1C(=O)N1CCN(Cc2ccc(-n3c(-c4cccnc4N)nc4cccnc43)cc2)CC1] | 4-(4-(4-(2-(2-aminopyridin-3-yl)-3H- imidazo[4,5-b]pyridin-3- yl)benzyl)piperazine-1- carbonyl)nicotinonitrile |
| 155 | 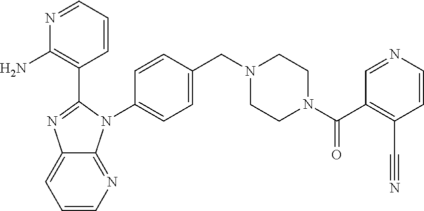 [SMILES: N#Cc1ccncc1C(=O)N1CCN(Cc2ccc(-n3c(-c4cccnc4N)nc4cccnc43)cc2)CC1] | 3-(4- (4-(2-(2-aminopyridin-3-yl)-3H- imidazo[4,5-b]pyridin-3- yl)benzyl)piperazine-1- carbonyl)isonicotinonitrile |
| 156 | 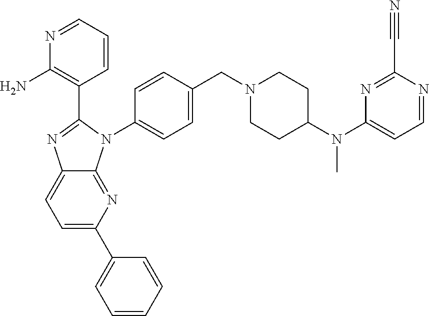 [SMILES: CN(c1ccnc(C#N)n1)C1CCN(Cc2ccc(-n3c(-c4cccnc4N)nc4ccc(-c5ccccc5)nc43)cc2)CC1] | 4-((1-(4-(2-(2-aminopyridin-3-yl)-5-phenyl- 3H-imidazo[4,5-b]pyridin-3- yl)benzyl)piperidin-4- yl)(methyl)amino)pyrimidine-2-carbonitrile |
| 157 | 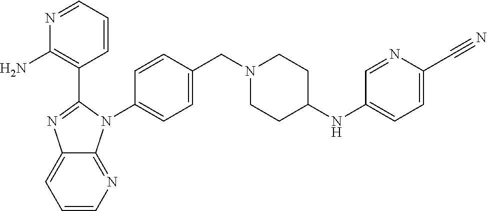 [SMILES: N#Cc1ccc(NC2CCN(Cc3ccc(-n4c(-c5cccnc5N)nc5cccnc54)cc3)CC2)cn1] | 5-((1-(4-(2-(2-aminopyridin-3-yl)-3H- imidazo[4,5-b]pyridin-3- yl)benzyl)piperidin-4- yl)amino)picolinonitrile |
| 158 | 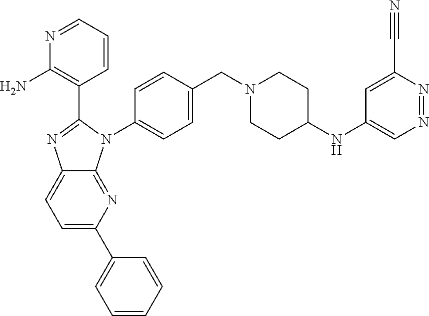 [SMILES: N#Cc1cc(NC2CCN(Cc3ccc(-n4c(-c5cccnc5N)nc5ccc(-c6ccccc6)nc54)cc3)CC2)cnn1] | 5-((1-(4-(2-(2-aminopyridin-3-yl)-5-phenyl- 3H-imidazo[4,5-b]pyridin-3- yl)benzyl)piperidin-4-yl)amino)pyridazine- 3-carbonitrile |
| 159 | 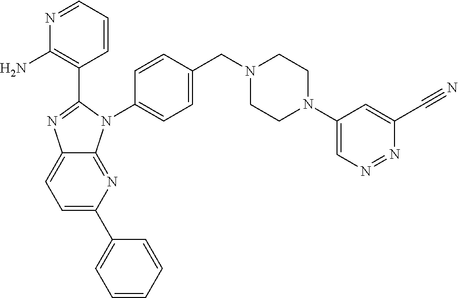 [SMILES: N#Cc1cc(N2CCN(Cc3ccc(-n4c(-c5cccnc5N)nc5ccc(-c6ccccc6)nc54)cc3)CC2)cnn1] | 5-(4-(4-(2-(2-aminopyridin-3-yl)-5-phenyl- 3H-imidazo[4,5-b]pyridin-3- yl)benzyl)piperazin-1-yl)pyridazine-3- carbonitrile |
| 160 | 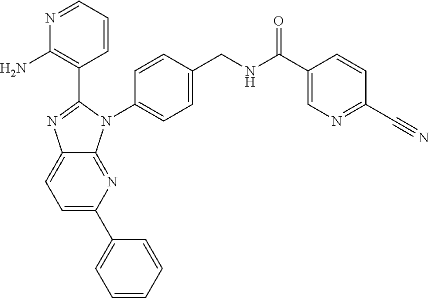 [SMILES: N#Cc1ccc(C(=O)NCc2ccc(-n3c(-c4cccnc4N)nc4ccc(-c5ccccc5)nc43)cc2)cn1] | N-(4-(2-(2-aminopyridin-3-yl)-5-phenyl- 3H-imidazo[4,5-b]pyridin-3-yl)benzyl)-6- cyanonicotinamide |
| 161 | 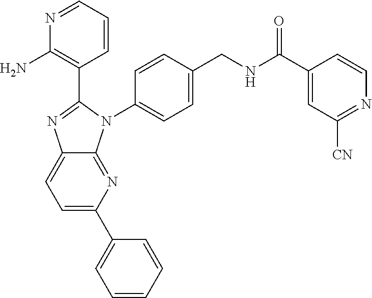 [SMILES: N#Cc1cc(C(=O)NCc2ccc(-n3c(-c4cccnc4N)nc4ccc(-c5ccccc5)nc43)cc2)ccn1] | N-(4-(2-(2-aminopyridin-3-yl)-5-phenyl- 3H-imidazo[4,5-b]pyridin-3-yl)benzyl)-2- cyanoisonicotinamide |
| 162 | 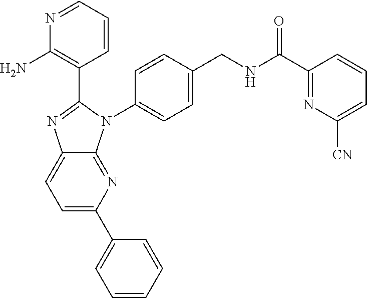 [SMILES: N#Cc1cccc(C(=O)NCc2ccc(-n3c(-c4cccnc4N)nc4ccc(-c5ccccc5)nc43)cc2)n1] | N-(4-(2-(2-aminopyridin-3-yl)-5-phenyl- 3H-imidazo[4,5-b]pyridin-3-yl)benzyl)-6- cyanopicolinamide |
| 163 | 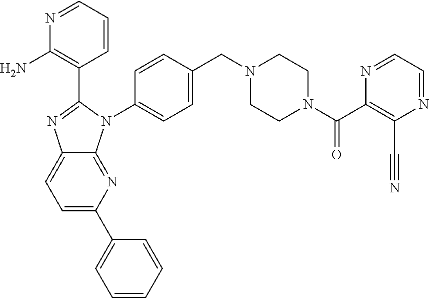 [SMILES: N#Cc1nccnc1C(=O)N1CCN(Cc2ccc(-n3c(-c4cccnc4N)nc4ccc(-c5ccccc5)nc43)cc2)CC1] | 3-(4-(4-(2-(2-aminopyridin-3-yl)-5-phenyl- 3H-imidazo[4,5-b]pyridin-3- yl)benzyl)piperazine-1-carbonyl)pyrazine- 2-carbonitrile |
| 164 | 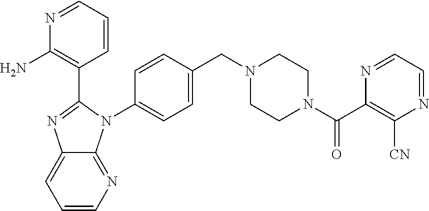 [SMILES: N#Cc1nccnc1C(=O)N1CCN(Cc2ccc(-n3c(-c4cccnc4N)nc4cccnc43)cc2)CC1] | 3-(4-(4-(2-(2-aminopyridin-3-yl)-3H- imidazo[4,5-b]pyridin-3- yl)benzyl)piperazine-1-carbonyl)pyrazine- 2-carbonitrile |
| 165 | 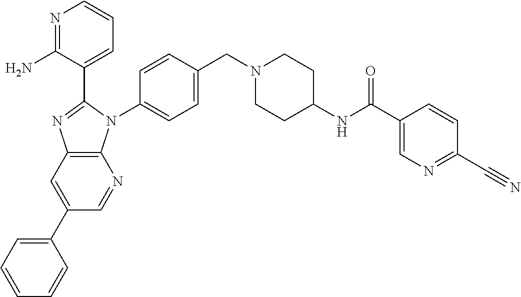 [SMILES: N#Cc1ccc(C(=O)NC2CCN(Cc3ccc(-n4c(-c5cccnc5N)nc5cc(-c6ccccc6)cnc54)cc3)CC2)cn1] | N-(1-(4-(2-(2-aminopyridin-3-yl)-6-phenyl- 3H-imidazo[4,5-b]pyridin-3- yl)benzyl)piperidin-4-yl)-6- cyanonicotinamide |
| 166 | 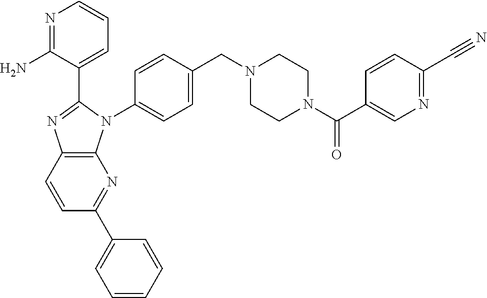 [SMILES: N#Cc1ccc(C(=O)N2CCN(Cc3ccc(-n4c(-c5cccnc5N)nc5ccc(-c6ccccc6)nc54)cc3)CC2)cn1] | 5-(4-(4-(2-(2-aminopyridin-3-yl)-5-phenyl- 3H-imidazo[4,5-b]pyridin-3- yl)benzyl)piperazine-1- carbonyl)picolinonitrile |
| 167 | 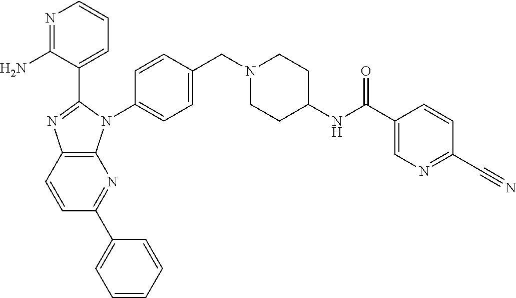 [SMILES: N#Cc1ccc(C(=O)NC2CCN(Cc3ccc(-n4c(-c5cccnc5N)nc5ccc(-c6ccccc6)nc54)cc3)CC2)cn1] | N-(1-(4-(2-(2-aminopyridin-3-yl)-5-phenyl- 3H-imidazo[4,5-b]pyridin-3- yl)benzyl)piperidin-4-yl)-6- cyanonicotinamide |
| 168 | 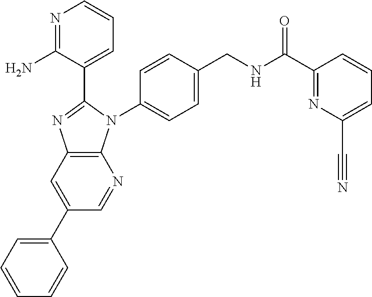 [SMILES: N#Cc1cccc(C(=O)NCc2ccc(-n3c(-c4cccnc4N)nc4cc(-c5ccccc5)cnc43)cc2)n1] | N-(4-(2-(2-aminopyridin-3-yl)-6-phenyl- 3H-imidazo[4,5-b]pyridin-3-yl)benzyl)-6- cyanopicolinamide |
| 169 | 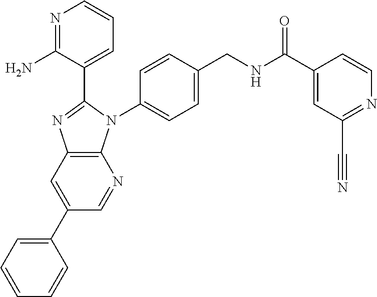 [SMILES: N#Cc1cc(C(=O)NCc2ccc(-n3c(-c4cccnc4N)nc4cc(-c5ccccc5)cnc43)cc2)ccn1] | N-(4-(2-(2-aminopyridin-3-yl)-6-phenyl- 3H-imidazo[4,5-b]pyridin-3-yl)benzyl)-2- cyanoisonicotinamide |
| 170 | 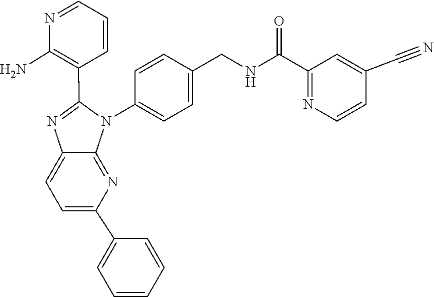 [SMILES: N#Cc1ccnc(C(=O)NCc2ccc(-n3c(-c4cccnc4N)nc4ccc(-c5ccccc5)nc43)cc2)c1] | N-(4-(2-(2-aminopyridin-3-yl)-5-phenyl- 3H-imidazo[4,5-b]pyridin-3-yl)benzyl)-4- cyanopicolinamide |
| 171 | 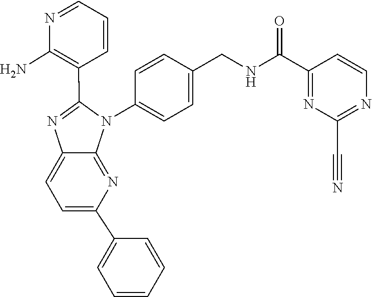 [SMILES: N#Cc1nccc(C(=O)NCc2ccc(-n3c(-c4cccnc4N)nc4ccc(-c5ccccc5)nc43)cc2)n1] | N-(4-(2-(2-aminopyridin-3-yl)-5-phenyl- 3H-imidazo[4,5-b]pyridin-3-yl)benzyl)- 2- cyanopyrimidine-4-carboxamide |
| 172 | 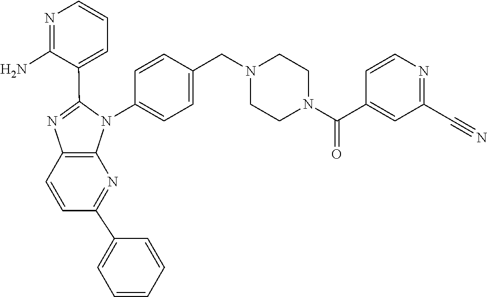 [SMILES: N#Cc1cc(C(=O)N2CCN(Cc3ccc(-n4c(-c5cccnc5N)nc5ccc(-c6ccccc6)nc54)cc3)CC2)ccn1] | 4-(4-(4-(2-(2-aminopyridin-3-yl)-5-phenyl- 3H-imidazo[4,5-b ]pyridin-3- yl)benzyl)piperazine-1- carbonyl)picolinonitrile |
| 173 | 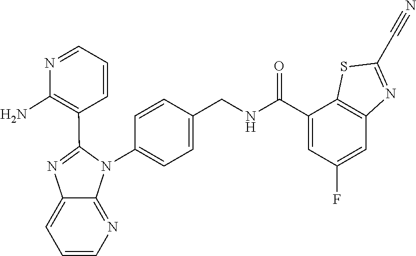 [SMILES: N#Cc1nc2cc(F)cc(C(=O)NCc3ccc(-n4c(-c5cccnc5N)nc5cccnc54)cc3)c2s1] | N-(4-(2-(2-aminopyridin-3-yl)-3H- imidazo[4,5-b]pyridin-3-yl)benzyl)-2- cyano-5-fluorobenzo[d]thiazole-7- carboxamide |
| 174 | 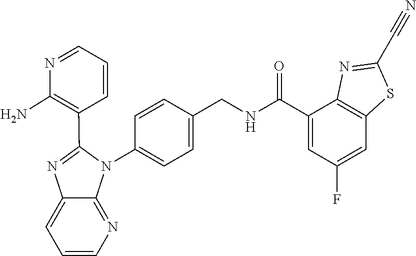 [SMILES: N#Cc1nc2c(C(=O)NCc3ccc(-n4c(-c5cccnc5N)nc5cccnc54)cc3)cc(F)cc2s1] | N-(4-(2-(2-aminopyridin-3-yl)-3H- imidazo[4,5-b]pyridin-3-yl)benzyl)-2- cyano-6-fluorobenzo[d]thiazole-4- carboxamide |
| 175 | 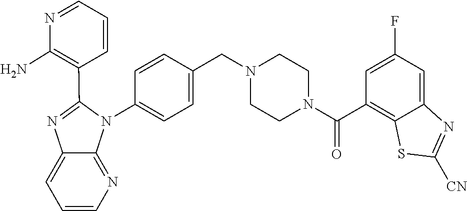 [SMILES: N#Cc1nc2cc(F)cc(C(=O)N3CCN(Cc4ccc(-n5c(-c6cccnc6N)nc6cccnc65)cc4)CC3)c2s1] | 7-(4-(4-(2-(2-aminopyridin-3-yl)-3H- imidazo[4,5-b]pyridin-3- yl)benzyl)piperazine-1-carbonyl)-5- fluorobenzo[d]thiazole-2-carbonitrile |
| 176 | 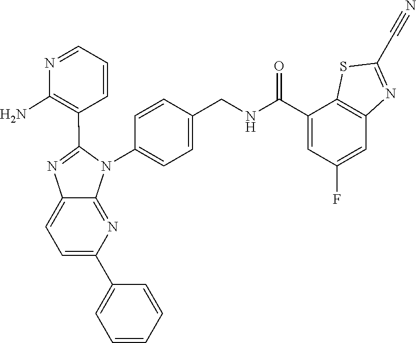 [SMILES: N#Cc1nc2cc(F)cc(C(=O)NCc3ccc(-n4c(-c5cccnc5N)nc5ccc(-c6ccccc6)nc54)cc3)c2s1] | N-(4-(2-(2-aminopyridin-3-yl)-5-phenyl- 3H-imidazo[4,5-b]pyridin-3-yl)benzyl)-2- cyano-5-fluorobenzo[d]thiazole-7- carboxamide |
| 177 | 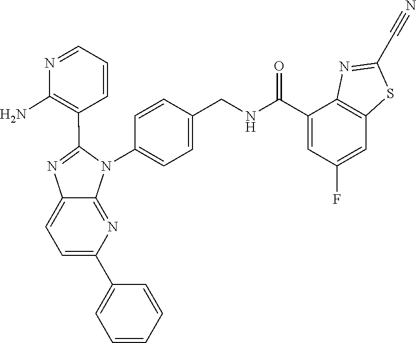 [SMILES: N#Cc1nc2c(C(=O)NCc3ccc(-n4c(-c5cccnc5N)nc5ccc(-c6ccccc6)nc54)cc3)cc(F)cc2s1] | N-(4-(2-(2-aminopyridin-3-yl)-5-phenyl- 3H-imidazo[4,5-b]pyridin-3-yl)benzyl)-2- cyano-6-fluorobenzo[d]thiazole-4- carboxamide |
| 178 | 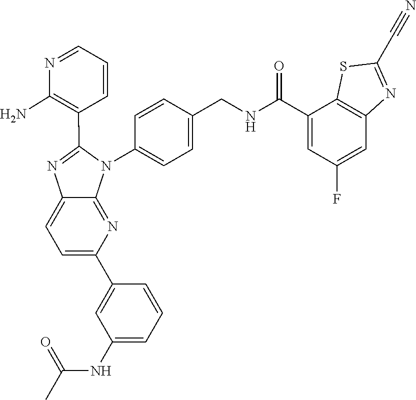 [SMILES: CC(=O)Nc1cccc(-c2ccc3nc(-c4cccnc4N)n(-c4ccc(CNC(=O)c5cc(F)cc6nc(C#N)sc56)cc4)c3n2)c1] | N-(4-(5-(3-acetamidophenyl)-2-(2- aminopyridin-3-yl)-3H-imidazo[4,5- b]pyridin-3-yl)benzyl)-2-cyano-5- fluorobenzo[d]thiazole-7-carboxamide |
| 179 | 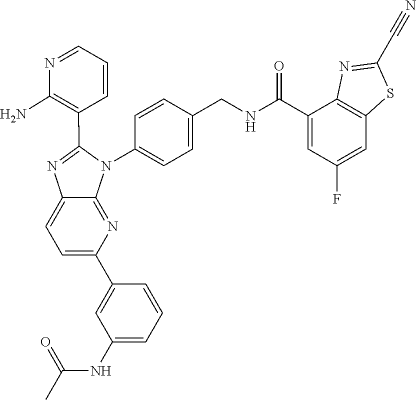 [SMILES: CC(=O)Nc1cccc(-c2ccc3nc(-c4cccnc4N)n(-c4ccc(CNC(=O)c5cc(F)cc6sc(C#N)nc56)cc4)c3n2)c1] | N-(4-(5-(3-acetamidophenyl)-2-(2- aminopyridin-3-yl)-3H-imidazo[4,5- b]pyridin-3-yl)benzyl)-2-cyano-6- fluorobenzo[d]thiazole-4-carboxamide |
| 180 | 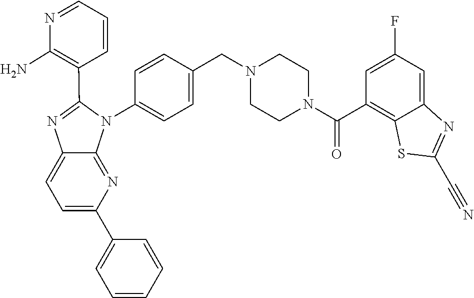 [SMILES: N#Cc1nc2cc(F)cc(C(=O)N3CCN(Cc4ccc(-n5c(-c6cccnc6N)nc6ccc(-c7ccccc7)nc65)cc4)CC3)c2s1] | 7-(4-(4-(2-(2-aminopyridin-3-yl)-5-phenyl- 3H-imidazo[4,5-b]pyridin-3- yl)benzyl)piperazine-1-carbonyl)-5- fluorobenzo[d]thiazole-2-carbonitrile |
| 181 | 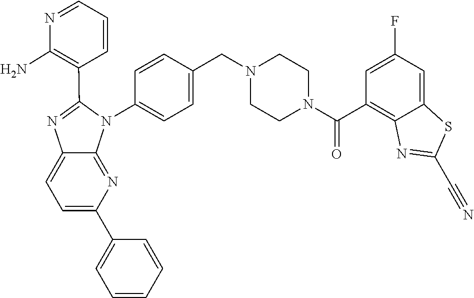 [SMILES: N#Cc1nc2c(C(=O)N3CCN(Cc4ccc(-n5c(-c6cccnc6N)nc6ccc(-c7ccccc7)nc65)cc4)CC3)cc(F)cc2s1] | 4-(4- (4-(2-(2-aminopyridin-3-yl)-5-phenyl- 3H-imidazo[4,5-b ]pyridin-3- yl)benzyl)piperazine-1-carbonyl)-6- fluorobenzo[d]thiazole-2-carbonitrile |
| 182 | 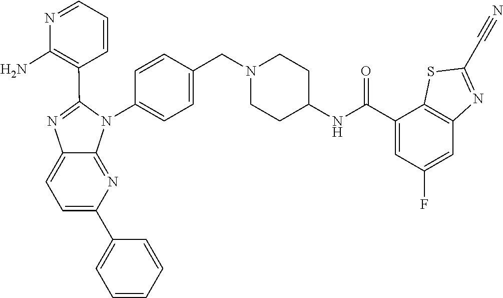 [SMILES: N#Cc1nc2cc(F)cc(C(=O)NC3CCN(Cc4ccc(-n5c(-c6cccnc6N)nc6ccc(-c7ccccc7)nc65)cc4)CC3)c2s1] | N-(1-(4-(2-(2-aminopyridin-3-yl)-5-phenyl- 3H-imidazo[4,5-b]pyridin-3- yl)benzyl)piperidin-4-yl)-2-cyano-5- fluorobenzo[d]thiazole-7-carboxamide |
| 183 | 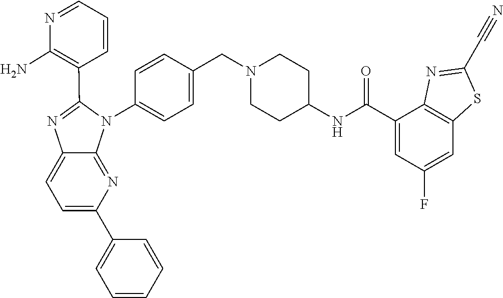 [SMILES: N#Cc1nc2c(C(=O)NC3CCN(Cc4ccc(-n5c(-c6cccnc6N)nc6ccc(-c7ccccc7)nc65)cc4)CC3)cc(F)cc2s1] | N-(1-(4-(2-(2-aminopyridin-3-yl)-5-phenyl- 3H-imidazo[4,5-b]pyridin-3- yl)benzyl)piperidin-4-yl)-2-cyano-6- fluorobenzo[d]thiazole-4-carboxamide |
| 184 | 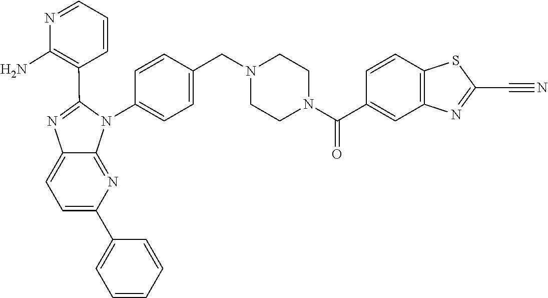 [SMILES: N#Cc1nc2cc(C(=O)N3CCN(Cc4ccc(-n5c(-c6cccnc6N)nc6ccc(-c7ccccc7)nc65)cc4)CC3)ccc2s1] | 5- (4-(4-(2-(2-aminopyridin-3-yl)-5-phenyl- 3H-imidazo[4,5-b]pyridin-3- yl)benzyl)piperazine-1- carbonyl)benzo[d]thiazole-2-carbonitrile |
| 185 | 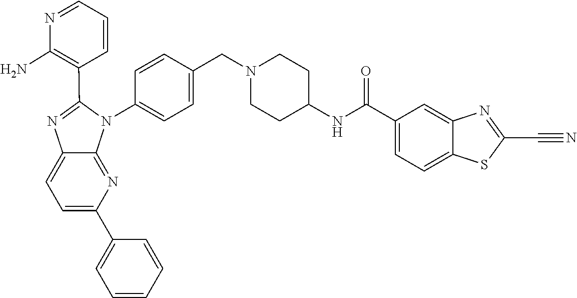 [SMILES: N#Cc1nc2cc(C(=O)NC3CCN(Cc4ccc(-n5c(-c6cccnc6N)nc6ccc(-c7ccccc7)nc65)cc4)CC3)ccc2s1] | N-(1-(4-(2-(2-aminopyridin-3-yl)-5-phenyl- 3H-imidazo[4,5-b ]pyridin-3- yl)benzyl)piperidin-4-yl)-2- cyanobenzo[d]thiazole-5-carboxamide |
| 186 | 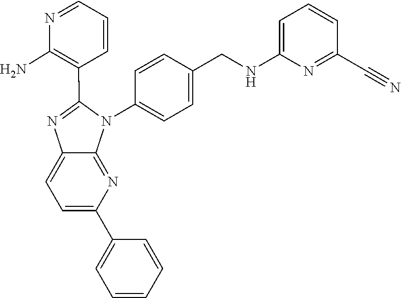 [SMILES: N#Cc1cccc(NCc2ccc(-n3c(-c4cccnc4N)nc4ccc(-c5ccccc5)nc43)cc2)n1] | 6-((4-(2-(2-aminopyridin-3-yl)-5-phenyl- 3H-imidazo[4,5-b]pyridin-3- yl)benzyl)amino)picolinonitrile |
| 187 | 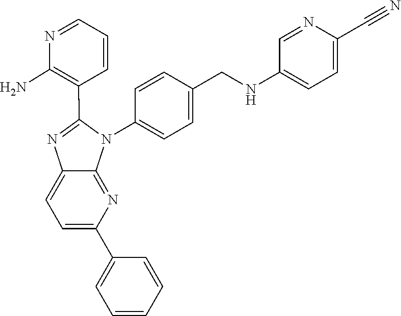 [SMILES: N#Cc1ccc(NCc2ccc(-n3c(-c4cccnc4N)nc4ccc(-c5ccccc5)nc43)cc2)cn1] | 5-((4-(2-(2-aminopyridin-3-yl)-5-phenyl- 3H-imidazo[4,5-b ]pyridin-3- yl)benzyl)amino)picolinonitrile |
| 188 | 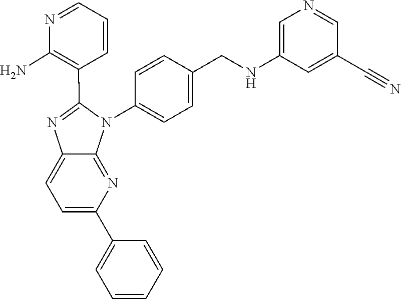 [SMILES: N#Cc1cncc(NCc2ccc(-n3c(-c4cccnc4N)nc4ccc(-c5ccccc5)nc43)cc2)c1] | 5-((4-(2-(2-aminopyridin-3-yl)-5-phenyl- 3H-imidazo[4,5-b]pyridin-3- yl)benzyl)amino)nicotinonitrile |
| 189 | 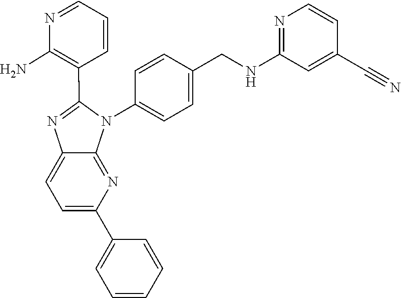 [SMILES: N#Cc1ccnc(NCc2ccc(-n3c(-c4cccnc4N)nc4ccc(-c5ccccc5)nc43)cc2)c1] | 2-((4-(2-(2-aminopyridin-3-yl)-5-phenyl- 3H-imidazo[4,5-b]pyridin-3- yl)benzyl)amino)isonicotinonitrile |
| 190 | 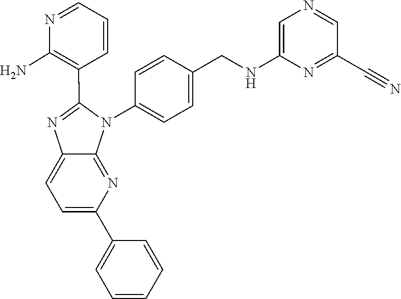 [SMILES: N#Cc1cncc(NCc2ccc(-n3c(-c4cccnc4N)nc4ccc(-c5ccccc5)nc43)cc2)n1] | 6-((4-(2-(2-aminopyridin-3-yl)-5-phenyl- 3H-imidazo[4,5-b]pyridin-3- yl)benzyl)amino)pyrazine-2-carbonitrile |
| 191 | 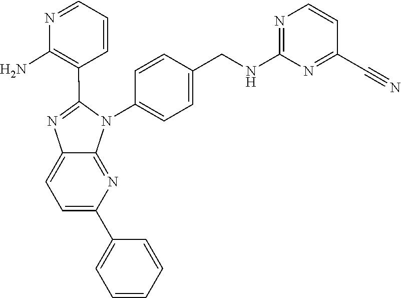 [SMILES: N#Cc1ccnc(NCc2ccc(-n3c(-c4cccnc4N)nc4ccc(-c5ccccc5)nc43)cc2)n1] | 2-((4-(2-(2-aminopyridin-3-yl)-5-phenyl- 3H-imidazo[4,5-b]pyridin-3- yl)benzyl)amino)pyrimidine-4-carbonitrile |
| 192 | 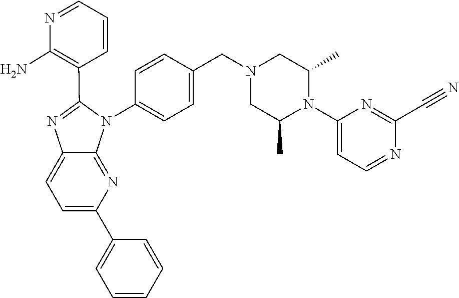 [SMILES: C[C@H]1CN(Cc2ccc(-n3c(-c4cccnc4N)nc4ccc(-c5ccccc5)nc43)cc2)C[C@H](C)N1c1ccnc(C#N)n1] | 4-((2S,6S)-4-(4-(2-(2-aminopyridin-3-yl)-5- phenyl-3H-imidazo[4,5-b]pyridin-3- yl)benzyl)-2,6-dimethylpiperazin-1- yl)pyrimidine-2-carbonitrile |
| 193 | 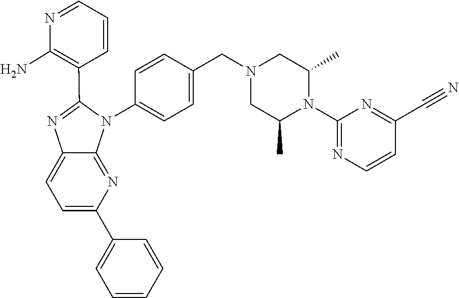 [SMILES: C[C@H]1CN(Cc2ccc(-n3c(-c4cccnc4N)nc4ccc(-c5ccccc5)nc43)cc2)C[C@H](C)N1c1nccc(C#N)n1] | 2-((2S,6S)-4-(4-(2-(2-aminopyridin-3-yl)-5- phenyl-3H-imidazo[4,5-b]pyridin-3- yl)benzyl)-2,6-dimethylpiperazin-1- yl)pyrimidine-4-carbonitrile |
| 194 | 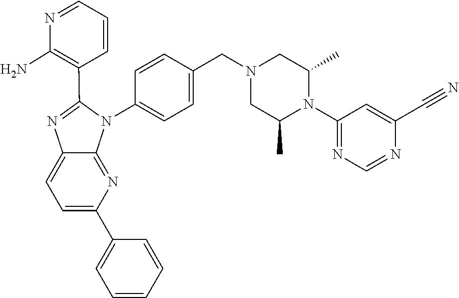 [SMILES: C[C@H]1CN(Cc2ccc(-n3c(-c4cccnc4N)nc4ccc(-c5ccccc5)nc43)cc2)C[C@H](C)N1c1cc(C#N)ncn1] | 6-((2S,6S)-4-(4-(2-(2-aminopyridin-3-yl)-5- phenyl-3H-imidazo[4,5-b]pyridin-3- yl)benzyl)-2,6-dimethylpiperazin-1- yl)pyrimidine-4-carbonitrile |
| 195 | 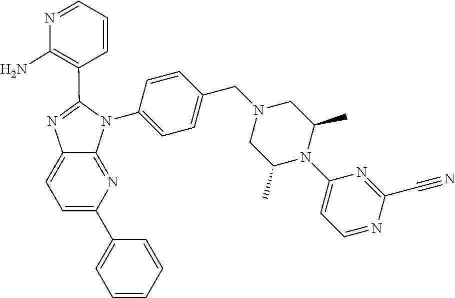 [SMILES: C[C@@H]1CN(Cc2ccc(-n3c(-c4cccnc4N)nc4ccc(-c5ccccc5)nc43)cc2)C[C@@H](C)N1c1ccnc(C#N)n1] | 4-((2R,6R)-4-(4-(2-(2-aminopyridin-3-yl)- 5-phenyl-3H-imidazo[4,5-b]pyridin-3- yl)benzyl)-2,6-dimethylpiperazin-1- yl)pyrimidine-2-carbonitrile |
| 196 | 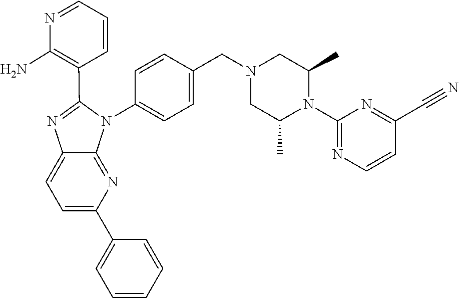 [SMILES: C[C@@H]1CN(Cc2ccc(-n3c(-c4cccnc4N)nc4ccc(-c5ccccc5)nc43)cc2)C[C@@H](C)N1c1nccc(C#N)n1] | 2-((2R,6R)-4-(4-(2-(2-aminopyridin-3-yl)- 5-phenyl-3H-imidazo[4,5-b]pyridin-3- yl)benzyl)-2,6-dimethylpiperazin-1- yl)pyrimidine-4-carbonitrile |
| 197 | 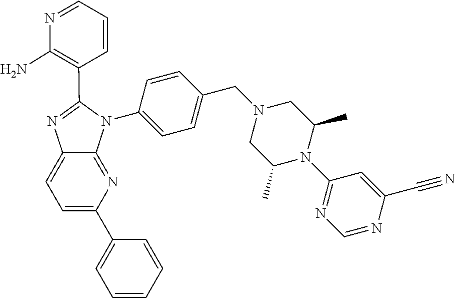 [SMILES: C[C@@H]1CN(Cc2ccc(-n3c(-c4cccnc4N)nc4ccc(-c5ccccc5)nc43)cc2)C[C@@H](C)N1c1cc(C#N)ncn1] | 6-((2R,6R)-4-(4-(2-(2-aminopyridin-3-yl)- 5-phenyl-3H-imidazo[4,5-b]pyridin-3- yl)benzyl)-2,6-dimethylpiperazin-1- yl)pyrimidine-4-carbonitrile |
| 198 | 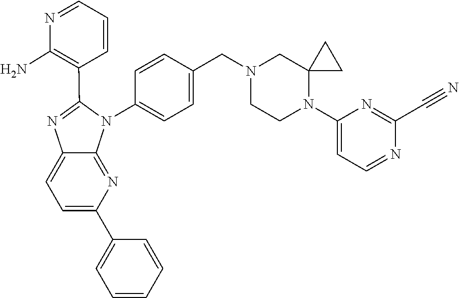 [SMILES: N#Cc1nccc(N2CCN(Cc3ccc(-n4c(-c5cccnc5N)nc5ccc(-c6ccccc6)nc54)cc3)CC23CC3)n1] | 4-(7-(4-(2-(2-aminopyridin-3-yl)-5-phenyl- 3H-imidazo[4,5-b]pyridin-3-yl)benzyl)-4,7- diazaspiro[2.5]octan-4-yl)pyrimidine-2- carbonitrile |
| 199 | 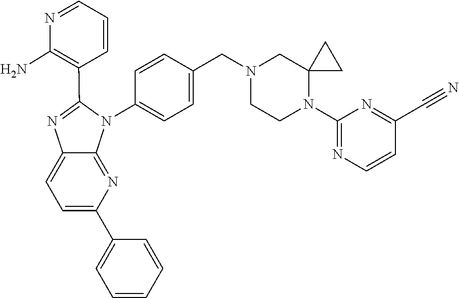 [SMILES: N#Cc1ccnc(N2CCN(Cc3ccc(-n4c(-c5cccnc5N)nc5ccc(-c6ccccc6)nc54)cc3)CC23CC3)n1] | 2-(7-(4-(2-(2-aminopyridin-3-yl)-5-phenyl- 3H-imidazo[4,5-b]pyridin-3-yl)benzyl)-4,7- diazaspiro[2.5]octan-4-yl)pyrimidine-4- carbonitrile |
| 200 | 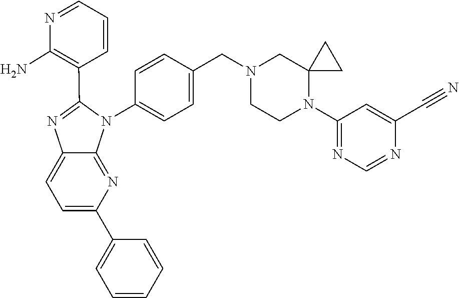 [SMILES: N#Cc1cc(N2CCN(Cc3ccc(-n4c(-c5cccnc5N)nc5ccc(-c6ccccc6)nc54)cc3)CC23CC3)ncn1] | 6-(7-(4-(2-(2-aminopyridin-3-yl)-5-phenyl- 3H-imidazo[4,5-b]pyridin-3-yl)benzyl)-4,7- diazaspiro[2.5]octan-4-yl)pyrimidine-4- carbonitrile |
| 201 | 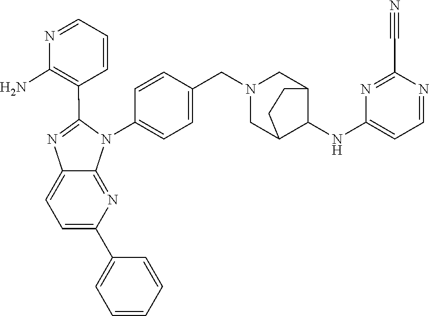 [SMILES: N#Cc1nccc(NC2C3CCC2CN(Cc2ccc(-n4c(-c5cccnc5N)nc5ccc(-c6ccccc6)nc54)cc2)C3)n1] | 4-((3-(4-(2-(2-aminopyridin-3-yl)-5-phenyl- 3H-imidazo[4,5-b]pyridin-3-yl)benzyl)-3- azabicyclo[3.2.1]octan-8- yl)amino)pyrimidine-2-carbonitrile |
| 202 | 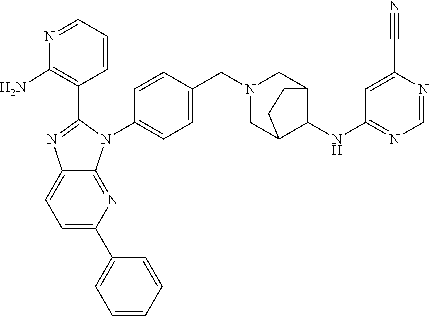 [SMILES: N#Cc1cc(NC2C3CCC2CN(Cc2ccc(-n4c(-c5cccnc5N)nc5ccc(-c6ccccc6)nc54)cc2)C3)ncn1] | 6-((3-(4-(2-(2-aminopyridin-3-yl)-5-phenyl- 3H-imidazo[4,5-b]pyridin-3-yl)benzyl)-3- azabicyclo[3.2.1]octan-8- yl)amino)pyrimidine-4-carbonitrile |
| 203 | 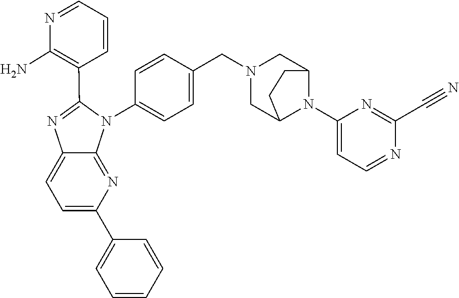 [SMILES: N#Cc1nccc(N2C3CCC2CN(Cc2ccc(-n4c(-c5cccnc5N)nc5ccc(-c6ccccc6)nc54)cc2)C3)n1] | 4-(3-(4-(2-(2-aminopyridin-3-yl)-5-phenyl- 3H-imidazo[4,5-b]pyridin-3-yl)benzyl)-3,8- diazabicyclo[3.2.1]octan-8-yl)pyrimidine-2- carbonitrile |
| 204 | 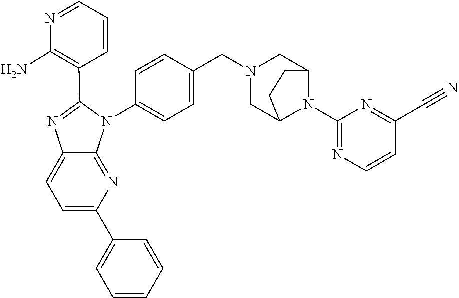 [SMILES: N#Cc1ccnc(N2C3CCC2CN(Cc2ccc(-n4c(-c5cccnc5N)nc5ccc(-c6ccccc6)nc54)cc2)C3)n1] | 2-(3-(4-(2-(2-aminopyridin-3-yl)-5-phenyl- 3H-imidazo[4,5-b]pyridin-3-yl)benzyl)-3,8- diazabicyclo[3.2.1]octan-8-yl)pyrimidine-4- carbonitrile |
| 205 | 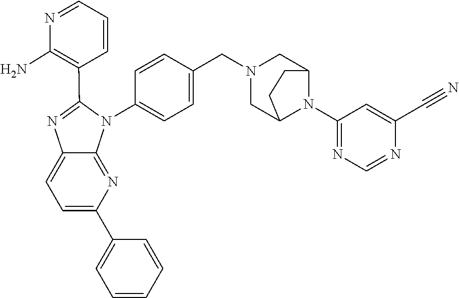 [SMILES: N#Cc1cc(N2C3CCC2CN(Cc2ccc(-n4c(-c5cccnc5N)nc5ccc(-c6ccccc6)nc54)cc2)C3)ncn1] | 6-(3-(4-(2-(2-aminopyridin-3-yl)-5-phenyl- 3H-imidazo[4,5-b]pyridin-3-yl)benzyl)-3,8- diazabicyclo[3.2.1 ]octan-8-yl)pyrimidine-4- carbonitrile |
| 206 | 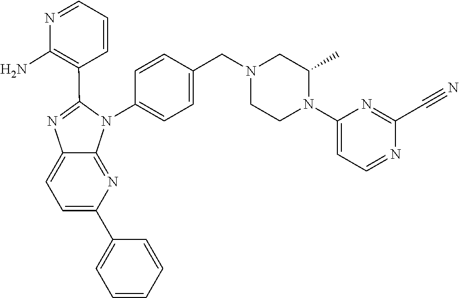 [SMILES: C[C@H]1CN(Cc2ccc(-n3c(-c4cccnc4N)nc4ccc(-c5ccccc5)nc43)cc2)CCN1c1ccnc(C#N)n1] | (S)-4-(4-(4-(2-(2-aminopyridin-3-yl)-5- phenyl-3H-imidazo[4,5-b]pyridin-3- yl)benzyl)-2-methylpiperazin-1- yl)pyrimidine-2-carbonitrile |
| 207 | 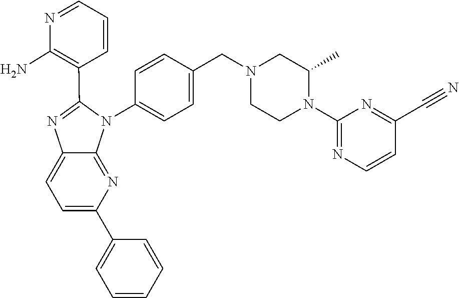 [SMILES: C[C@H]1CN(Cc2ccc(-n3c(-c4cccnc4N)nc4ccc(-c5ccccc5)nc43)cc2)CCN1c1nccc(C#N)n1] | (S)-2-(4-(4-(2-(2-aminopyridin-3-yl)-5- phenyl-3H-imidazo[4,5-b]pyridin-3- yl)benzyl)-2-methylpiperazin-1- yl)pyrimidine-4-carbonitrile |
| 208 | 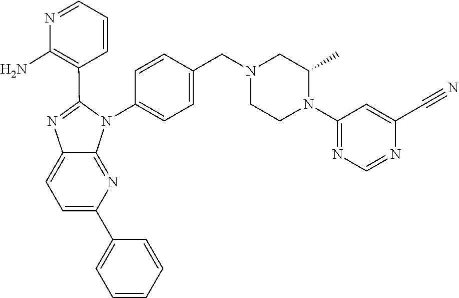 [SMILES: C[C@H]1CN(Cc2ccc(-n3c(-c4cccnc4N)nc4ccc(-c5ccccc5)nc43)cc2)CCN1c1cc(C#N)ncn1] | (S)-6-(4-(4-(2-(2-aminopyridin-3-yl)-5- phenyl-3H-imidazo[4,5-b]pyridin-3- yl)benzyl)-2-methylpiperazin-1- yl)pyrimidine-4-carbonitrile |
| 209 | 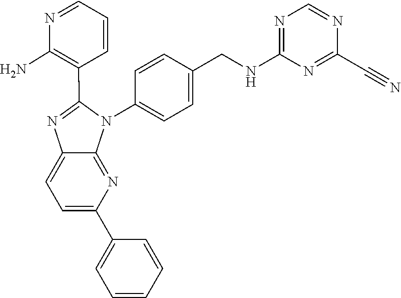 [SMILES: N#Cc1ncnc(NCc2ccc(-n3c(-c4cccnc4N)nc4ccc(-c5ccccc5)nc43)cc2)n1] | 4-((4-(2-(2-aminopyridin-3-yl)-5-phenyl- 3H-imidazo[4,5-b]pyridin-3- yl)benzyl)amino)-1,3,5-triazine-2- carbonitrile |
| 210 | 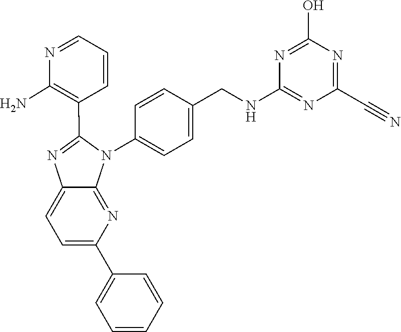 [SMILES: N#Cc1nc(O)nc(NCc2ccc(-n3c(-c4cccnc4N)nc4ccc(-c5ccccc5)nc43)cc2)n1] | 4-((4-(2-(2-aminopyridin-3-yl)-5-phenyl- 3H-imidazo[4,5-b ]pyridin-3- yl)benzyl)amino)-6-hydroxy-1,3,5-triazine- 2-carbonitrile |
| 211 | 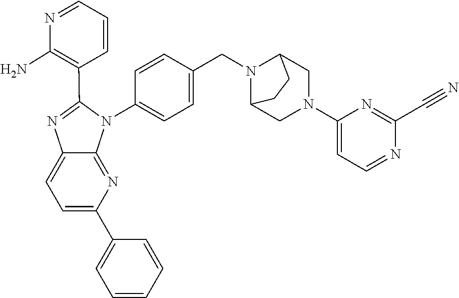 [SMILES: N#Cc1nccc(N2CC3CCC(C2)N3Cc2ccc(-n3c(-c4cccnc4N)nc4ccc(-c5ccccc5)nc43)cc2)n1] | 4-(8-(4-(2-(2-aminopyridin-3-yl)-5-phenyl- 3H-imidazo[4,5-b]pyridin-3-yl)benzyl)-3,8- diazabicyclo[3.2.1 ]octan-3-yl)pyrimidine-2- carbonitrile |
| 212 | 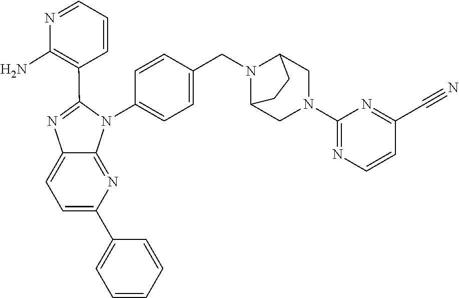 [SMILES: N#Cc1ccnc(N2CC3CCC(C2)N3Cc2ccc(-n3c(-c4cccnc4N)nc4ccc(-c5ccccc5)nc43)cc2)n1] | 2-(8-(4-(2-(2-aminopyridin-3-yl)-5-phenyl- 3H-imidazo[4,5-b]pyridin-3-yl)benzyl)-3,8- diazabicyclo[3.2.1]octan-3-yl)pyrimidine-4- carbonitrile |
| 213 | 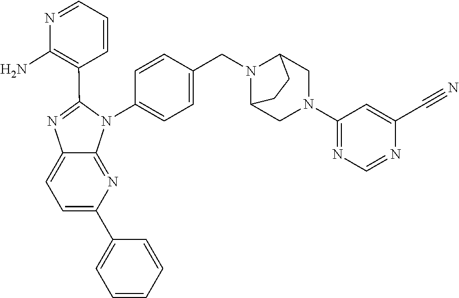 [SMILES: N#Cc1cc(N2CC3CCC(C2)N3Cc2ccc(-n3c(-c4cccnc4N)nc4ccc(-c5ccccc5)nc43)cc2)ncn1] | 6-(8-(4-(2-(2-aminopyridin-3-yl)-5-phenyl- 3H-imidazo[4,5-b]pyridin-3-yl)benzyl)-3,8- diazabicyclo[3.2.1 ]octan-3-yl)pyrimidine-4- carbonitrile |
| 214 | 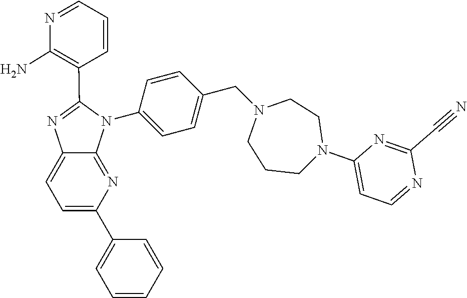 [SMILES: N#Cc1nccc(N2CCCN(Cc3ccc(-n4c(-c5cccnc5N)nc5ccc(-c6ccccc6)nc54)cc3)CC2)n1] | 4-(4-(4-(2-(2-aminopyridin-3-yl)-5-phenyl- 3H-imidazo[4,5-b]pyridin-3-yl)benzyl)-1,4- diazepan-1-yl)pyrimidine-2-carbonitrile |
| 215 | 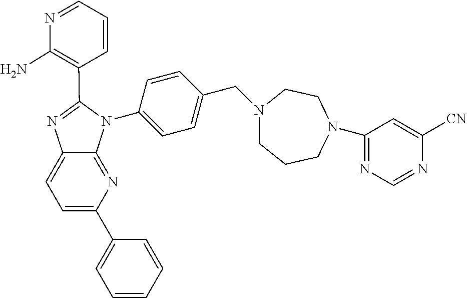 [SMILES: N#Cc1cc(N2CCCN(Cc3ccc(-n4c(-c5cccnc5N)nc5ccc(-c6ccccc6)nc54)cc3)CC2)ncn1] | 6-(4-(4-(2-(2-aminopyridin-3-yl)-5-phenyl- 3H-imidazo[4,5-b]pyridin-3-yl)benzyl)-1,4- diazepan-1-yl)pyrimidine-4-carbonitrile |
| 216 | 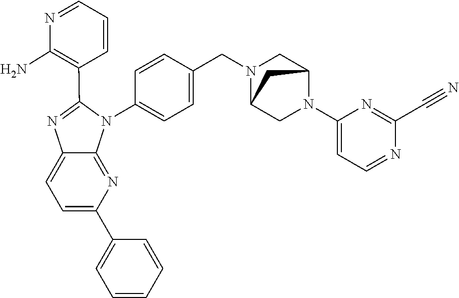 [SMILES: N#Cc1nccc(N2C[C@H]3C[C@@H]2CN3Cc2ccc(-n3c(-c4cccnc4N)nc4ccc(-c5ccccc5)nc43)cc2)n1] | 4-((1R,4R)-5-(4-(2-(2-aminopyridin-3-yl)- 5-phenyl-3H-imidazo[4,5-b]pyridin-3- yl)benzyl)-2,5-diazabicyclo[2.2.1]heptan-2- yl)pyrimidine-2-carbonitrile |
| 217 | 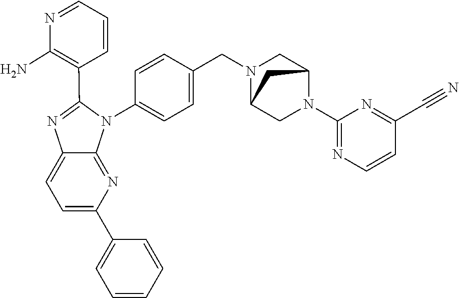 [SMILES: N#Cc1ccnc(N2C[C@H]3C[C@@H]2CN3Cc2ccc(-n3c(-c4cccnc4N)nc4ccc(-c5ccccc5)nc43)cc2)n1] | 2-((1R,4R)-5-(4-(2-(2-aminopyridin-3-yl)- 5-phenyl-3H-imidazo[4,5-b]pyridin-3- yl)benzyl)-2,5-diazabicyclo[2.2.1]heptan-2- yl)pyrimidine-4-carbonitrile |
| 218 | 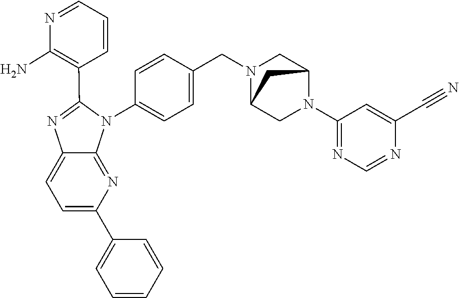 [SMILES: N#Cc1cc(N2C[C@H]3C[C@@H]2CN3Cc2ccc(-n3c(-c4cccnc4N)nc4ccc(-c5ccccc5)nc43)cc2)ncn1] | 6-((1R,4R)-5-(4-(2-(2-aminopyridin-3-yl)- 5-phenyl-3H-imidazo[4,5-b]pyridin-3- yl)benzyl)-2,5-diazabicyclo[2.2.1]heptan-2- yl)pyrimidine-4-carbonitrile |
| 219 | 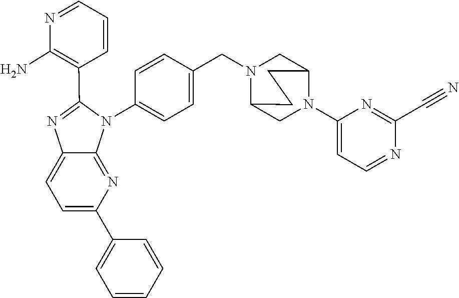 [SMILES: N#Cc1nccc(N2CC3CCC2CN3Cc2ccc(-n3c(-c4cccnc4N)nc4ccc(-c5ccccc5)nc43)cc2)n1] | 4-(5- (4-(2-(2-aminopyridin-3-yl)-5-phenyl- 3H-imidazo[4,5-b]pyridin-3-yl)benzyl)-2,5- diazabicyclo[2.2.2]octan-2-yl)pyrimidine-2- |
| 220 | 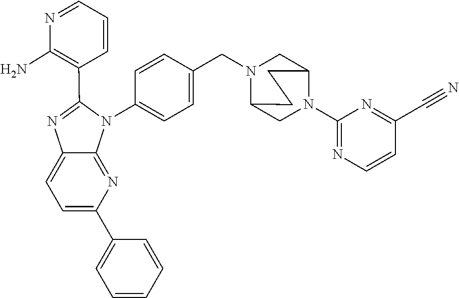 [SMILES: N#Cc1ccnc(N2CC3CCC2CN3Cc2ccc(-n3c(-c4cccnc4N)nc4ccc(-c5ccccc5)nc43)cc2)n1] | 2-(5-(4-(2-(2-aminopyridin-3-yl)-5-phenyl- 3H-imidazo[4,5-b]pyridin-3-yl)benzyl)-2,5- diazabicyclo[2.2.2]octan-2-yl)pyrimidine-4- carbonitrile |
| 221 | 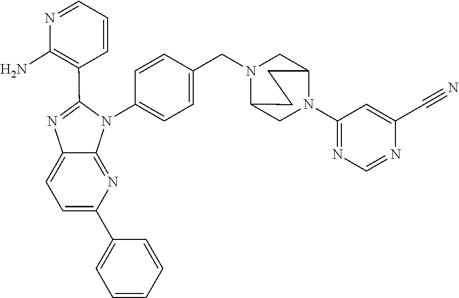 [SMILES: N#Cc1cc(N2CC3CCC2CN3Cc2ccc(-n3c(-c4cccnc4N)nc4ccc(-c5ccccc5)nc43)cc2)ncn1] | 6-(5-(4-(2-(2-aminopyridin-3-yl)-5-phenyl- 3H-imidazo[4,5-b]pyridin-3-yl)benzyl)-2,5- diazabicyclo[2.2.2]octan-2-yl)pyrimidine-4- carbonitrile |
| 222 | 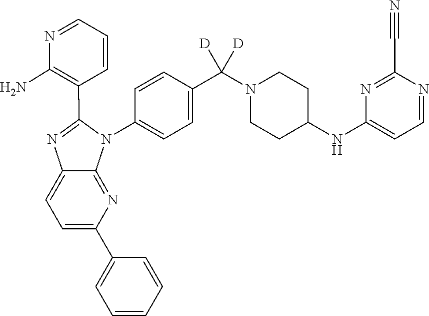 [SMILES: [2H]C([2H])(c1ccc(-n2c(-c3cccnc3N)nc3ccc(-c4ccccc4)nc32)cc1)N1CCC(Nc2ccnc(C#N)n2)CC1] | 4-((1-((4-(2-(2-aminopyridin-3-yl)-5- phenyl-3H-imidazo[4,5-b]pyridin-3- yl)phenyl)methyl-d2)piperidin-4- yl)amino)pyrimidine-2-carbonitrile |
| 223 | 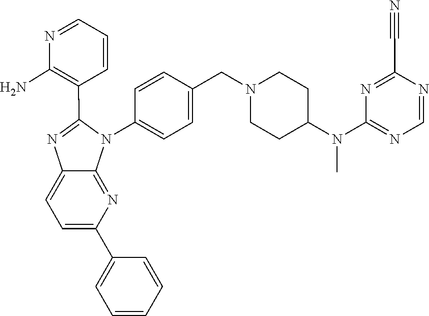 [SMILES: CN(c1ncnc(C#N)n1)C1CCN(Cc2ccc(-n3c(-c4cccnc4N)nc4ccc(-c5ccccc5)nc43)cc2)CC1] | 4-((1-(4-(2-(2-aminopyridin-3-yl)-5-phenyl- 3H-imidazo[4,5-b]pyridin-3- yl)benzyl)piperidin-4-yl)(methyl)amino)- 1,3,5-triazine-2-carbonitrile |
| 224 | 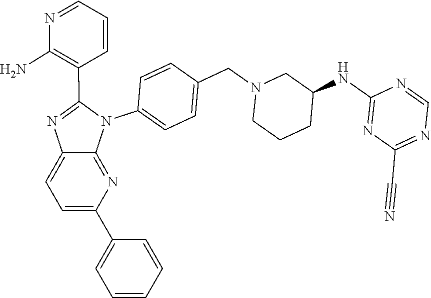 [SMILES: N#Cc1ncnc(N[C@H]2CCCN(Cc3ccc(-n4c(-c5cccnc5N)nc5ccc(-c6ccccc6)nc54)cc3)C2)n1] | (S)-4-((1-(4-(2-(2-aminopyridin-3-yl)-5- phenyl-3H-imidazo[4,5-b]pyridin-3- yl)benzyl)piperidin-3-yl)amino)-1,3,5- triazine-2-carbonitrile |
| 225 | 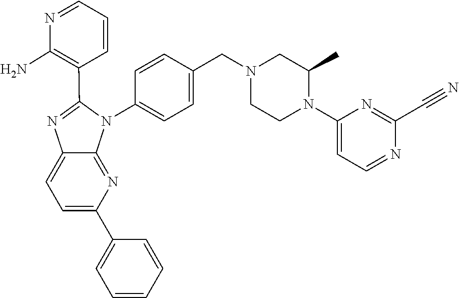 [SMILES: C[C@@H]1CN(Cc2ccc(-n3c(-c4cccnc4N)nc4ccc(-c5ccccc5)nc43)cc2)CCN1c1ccnc(C#N)n1] | (R)-4-(4-(4-(2-(2-aminopyridin-3-yl)-5- phenyl-3H-imidazo[4,5-b]pyridin-3- yl)benzyl)-2-methylpiperazin-1- yl)pyrimidine-2-carbonitrile |
| 226 | 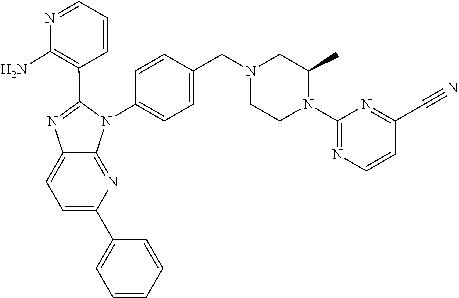 [SMILES: C[C@@H]1CN(Cc2ccc(-n3c(-c4cccnc4N)nc4ccc(-c5ccccc5)nc43)cc2)CCN1c1nccc(C#N)n1] | (R)-2-(4-(4-(2-(2-aminopyridin-3-yl)-5- phenyl-3H-imidazo[4,5-b]pyridin-3- yl)benzyl)-2-methylpiperazin-1- yl)pyrimidine-4-carbonitrile |
| 227 | 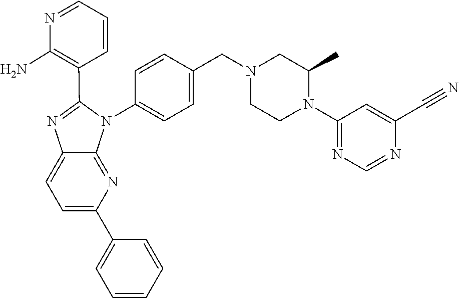 [SMILES: C[C@@H]1CN(Cc2ccc(-n3c(-c4cccnc4N)nc4ccc(-c5ccccc5)nc43)cc2)CCN1c1cc(C#N)ncn1] | (R)-6-(4-(4-(2-(2-aminopyridin-3-yl)-5- phenyl-3H-imidazo[4,5-b]pyridin-3- yl)benzyl)-2-methylpiperazin-1- yl)pyrimidine-4-carbonitrile |
| 228 | 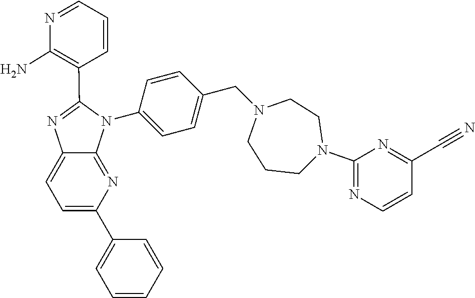 [SMILES: N#Cc1ccnc(N2CCCN(Cc3ccc(-n4c(-c5cccnc5N)nc5ccc(-c6ccccc6)nc54)cc3)CC2)n1] | 2-(4-(4-(2-(2-aminopyridin-3-yl)-5-phenyl- 3H-imidazo[4,5-b]pyridin-3-yl)benzyl)-1,4- diazepan-1-yl)pyrimidine-4-carbonitrile |
| 229 | 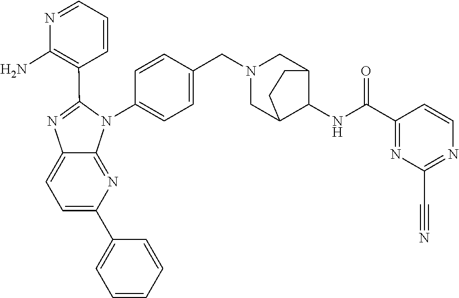 [SMILES: N#Cc1nccc(C(=O)NC2C3CCC2CN(Cc2ccc(-n4c(-c5cccnc5N)nc5ccc(-c6ccccc6)nc54)cc2)C3)n1] | N-(3-(4-(2-(2-aminopyridin-3-yl)-5-phenyl- 3H-imidazo[4,5-b]pyridin-3-yl)benzyl)-3- azabicyclo[3.2.1]octan-8-yl)-2- cyanopyrimidine-4-carboxamide |
| 230 | 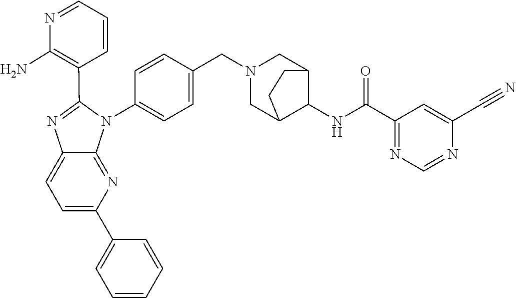 [SMILES: N#Cc1cc(C(=O)NC2C3CCC2CN(Cc2ccc(-n4c(-c5cccnc5N)nc5ccc(-c6ccccc6)nc54)cc2)C3)ncn1] | N-(3-(4-(2-(2-aminopyridin-3-yl)-5-phenyl- 3H-imidazo[4,5-b]pyridin-3-yl)benzyl)-3- azabicyclo[3.2.1]octan-8-yl)-6- cyanopyrimidine-4-carboxamide |
| 231 | 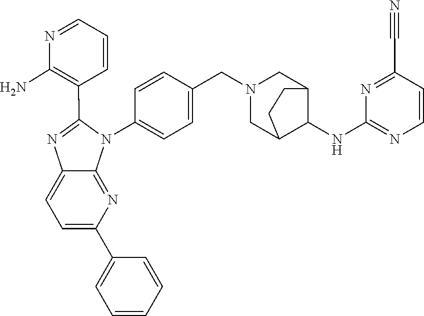 [SMILES: N#Cc1ccnc(NC2C3CCC2CN(Cc2ccc(-n4c(-c5cccnc5N)nc5ccc(-c6ccccc6)nc54)cc2)C3)n1] | 2-((3-(4-(2-(2-aminopyridin-3-yl)-5-phenyl- 3H-imidazo[4,5-b]pyridin-3-yl)benzyl)-3- azabicyclo[3.2.1]octan-8- yl)amino)pyrimidine-4-carbonitrile |
| 232 | 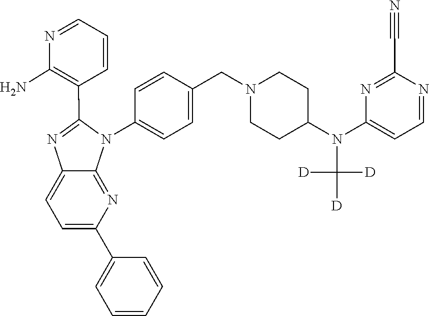 [SMILES: [2H]C([2H])([2H])N(c1ccnc(C#N)n1)C1CCN(Cc2ccc(-n3c(-c4cccnc4N)nc4ccc(-c5ccccc5)nc43)cc2)CC1] | 4-((1-(4-(2-(2-aminopyridin-3-yl)-5-phenyl- 3H-imidazo[4,5-b]pyridin-3- yl)benzyl)piperidin-4-yl)(methyl- d3)amino)pyrimidine-2-carbonitrile |
| 233 | 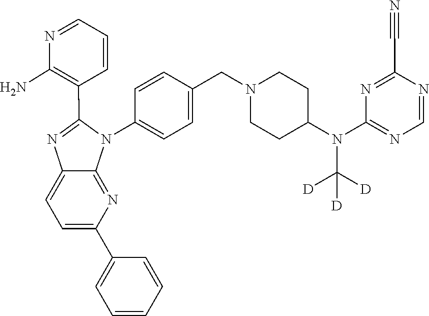 [SMILES: [2H]C([2H])([2H])N(c1ncnc(C#N)n1)C1CCN(Cc2ccc(-n3c(-c4cccnc4N)nc4ccc(-c5ccccc5)nc43)cc2)CC1] | 4-((1-(4-(2-(2-aminopyridin-3-yl)-5-phenyl- 3H-imidazo[4,5-b]pyridin-3- yl)benzyl)piperidin-4-yl)(methyl- d3)amino)-1,3,5-triazine-2-carbonitrile |
| 234 | 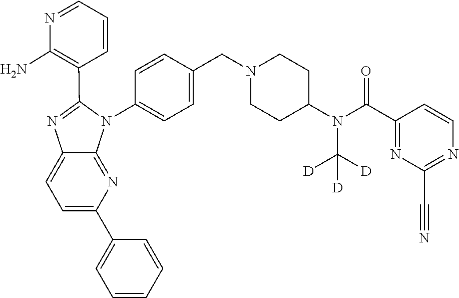 [SMILES: [2H]C([2H])([2H])N(C(=O)c1ccnc(C#N)n1)C1CCN(Cc2ccc(-n3c(-c4cccnc4N)nc4ccc(-c5ccccc5)nc43)cc2)CC1] | N-(1-(4-(2-(2-aminopyridin-3-yl)-5-phenyl- 3H-imidazo[4,5-b]pyridin-3- yl)benzyl)piperidin-4-yl)-2-cyano-N- (methyl-d3)pyrimidine-4-carboxamide |
| 235 | 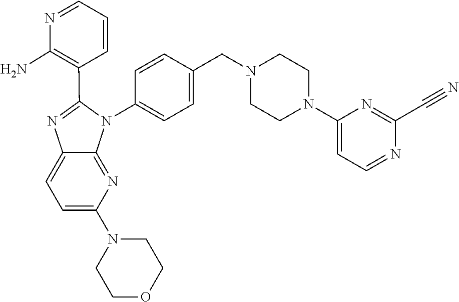 [SMILES: N#Cc1nccc(N2CCN(Cc3ccc(-n4c(-c5cccnc5N)nc5ccc(N6CCOCC6)nc54)cc3)CC2)n1] | 4-(4-(4-(2-(2-aminopyridin-3-yl)-5- morpholino-3H-imidazo[4,5-b]pyridin-3- yl)benzyl)piperazin-1-yl)pyrimidine-2- carbonitrile |
| 236 | 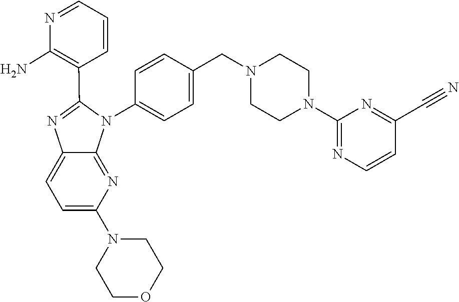 [SMILES: N#Cc1ccnc(N2CCN(Cc3ccc(-n4c(-c5cccnc5N)nc5ccc(N6CCOCC6)nc54)cc3)CC2)n1] | 2-(4-(4-(2-(2-aminopyridin-3-yl)-5- morpholino-3H-imidazo[4,5-b]pyridin-3- yl)benzyl)piperazin-1-yl)pyrimidine-4- carbonitrile |
| 237 | 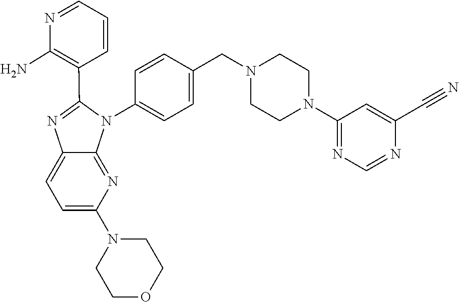 [SMILES: N#Cc1cc(N2CCN(Cc3ccc(-n4c(-c5cccnc5N)nc5ccc(N6CCOCC6)nc54)cc3)CC2)ncn1] | 6-(4-(4-(2-(2-aminopyridin-3-yl)-5- morpholino-3H-imidazo[4,5-b]pyridin-3- yl)benzyl)piperazin-1-yl)pyrimidine-4- carbonitrile |
| 238 | 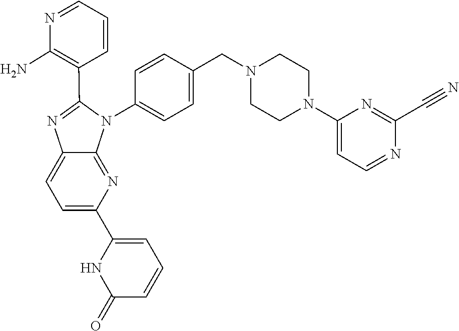 [SMILES: N#Cc1nccc(N2CCN(Cc3ccc(-n4c(-c5cccnc5N)nc5ccc(-c6cccc(=O)[nH]6)nc54)cc3)CC2)n1] | 4-(4-(4-(2-(2-aminopyridin-3-yl)-5-(6-oxo- 1,6-dihydropyridin-2-yl)-3H-imidazo[4,5- b]pyridin-3-yl)benzyl)piperazin-1- yl)pyrimidine-2-carbonitrile |
| 239 | 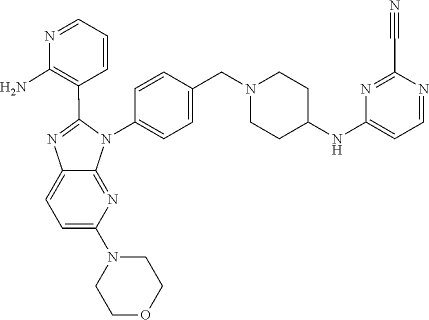 [SMILES: N#Cc1nccc(NC2CCN(Cc3ccc(-n4c(-c5cccnc5N)nc5ccc(N6CCOCC6)nc54)cc3)CC2)n1] | 4-((1-(4-(2-(2-aminopyridin-3-yl)-5- morpholino-3H-imidazo[4,5-b]pyridin-3- yl)benzyl)piperidin-4-yl)amino)pyrimidine- 2-carbonitrile |
| 240 | 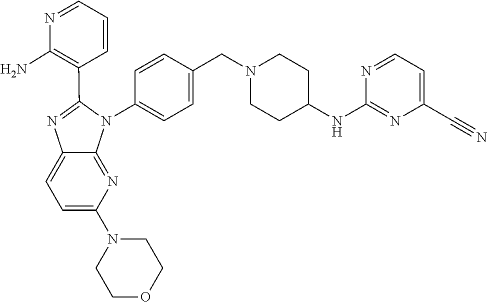 [SMILES: N#Cc1ccnc(NC2CCN(Cc3ccc(-n4c(-c5cccnc5N)nc5ccc(N6CCOCC6)nc54)cc3)CC2)n1] | 2-((1-(4-(2-(2-aminopyridin-3-yl)-5- morpholino-3H-imidazo[4,5-b]pyridin-3- yl)benzyl)piperidin-4-yl)amino)pyrimidine- 4-carbonitrile |
| 241 | 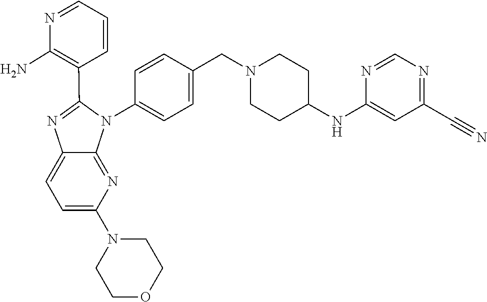 [SMILES: N#Cc1cc(NC2CCN(Cc3ccc(-n4c(-c5cccnc5N)nc5ccc(N6CCOCC6)nc54)cc3)CC2)ncn1] | 6-((1-(4-(2-(2-aminopyridin-3-yl)-5- morpholino-3H-imidazo[4,5-b]pyridin-3- yl)benzyl)piperidin-4-yl)amino)pyrimidine- 4-carbonitrile |
| 242 | 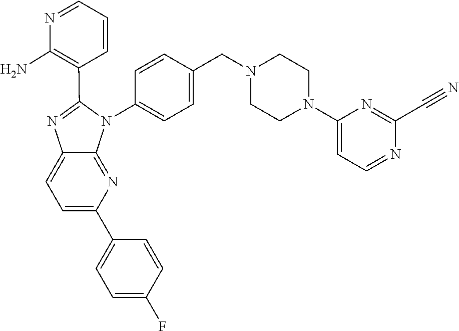 [SMILES: N#Cc1nccc(N2CCN(Cc3ccc(-n4c(-c5cccnc5N)nc5ccc(-c6ccc(F)cc6)nc54)cc3)CC2)n1] | 4.(4- (4-(2-(2-aminopyridin-3-yl)-5-(4- fluorophenyl)-3H-imidazo[4,5-b]pyridin-3- yl)benzyl)piperazin-1-yl)pyrimidine-2- carbonitrile |
| 243 | 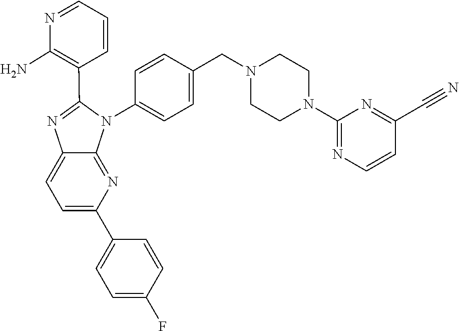 [SMILES: N#Cc1ccnc(N2CCN(Cc3ccc(-n4c(-c5cccnc5N)nc5ccc(-c6ccc(F)cc6)nc54)cc3)CC2)n1] | 2-(4-(4-(2-(2-aminopyridin-3-yl)-5-(4- fluorophenyl)-3H-imidazo[4,5-b]pyridin-3- yl)benzyl)piperazin-1-yl)pyrimidine-4- carbonitrile |
| 244 | 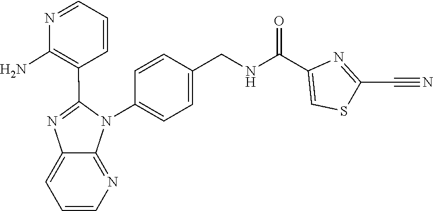 [SMILES: N#Cc1nc(C(=O)NCc2ccc(-n3c(-c4cccnc4N)nc4cccnc43)cc2)cs1] | N-(4-(2-(2-aminopyridin-3-yl)-3H- imidazo[4,5-b]pyridin-3-yl)benzyl)-2- cyanothiazole-4-carboxamide |
| 245 | 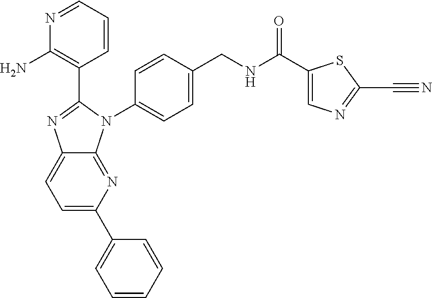 [SMILES: N#Cc1ncc(C(=O)NCc2ccc(-n3c(-c4cccnc4N)nc4ccc(-c5ccccc5)nc43)cc2)s1] | N-(4-(2-(2-aminopyridin-3-yl)-5-phenyl- 3H-imidazo[4,5-b]pyridin-3-yl)benzyl)-2- cyanothiazole-5-carboxamide |
| 246 | 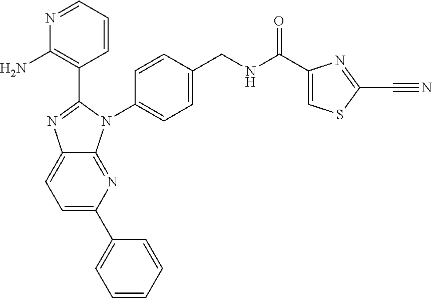 [SMILES: N#Cc1nc(C(=O)NCc2ccc(-n3c(-c4cccnc4N)nc4ccc(-c5ccccc5)nc43)cc2)cs1] | N-(4-(2-(2-aminopyridin-3-yl)-5-phenyl- 3H-imidazo[4,5-b]pyridin-3-yl)benzyl)-2- cyanothiazole-4-carboxamide |
| 247 | 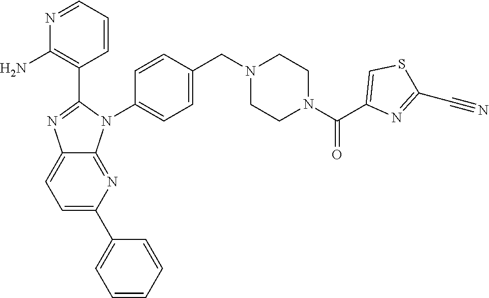 [SMILES: N#Cc1nc(C(=O)N2CCN(Cc3ccc(-n4c(-c5cccnc5N)nc5ccc(-c6ccccc6)nc54)cc3)CC2)cs1] | 4-(4-(4-(2-(2-aminopyridin-3-yl)-5-phenyl- 3H-imidazo[4,5-b]pyridin-3- yl)benzyl)piperazine-1-carbonyl)thiazole-2- carbonitrile |
| 248 | 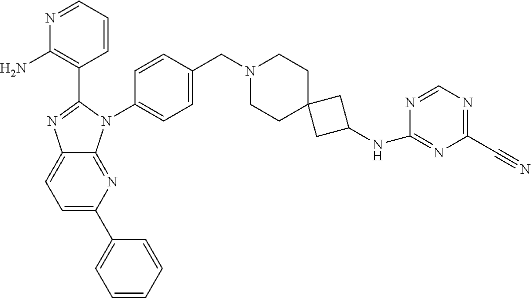 [SMILES: N#Cc1ncnc(NC2CC3(CCN(Cc4ccc(-n5c(-c6cccnc6N)nc6ccc(-c7ccccc7)nc65)cc4)CC3)C2)n1] | 4-((7-(4-(2-(2-aminopyridin-3-yl)-5-phenyl- 3H-imidazo[4,5-b]pyridin-3-yl)benzyl)-7- azaspiro[3.5]nonan-2-yl)amino)-1,3,5- triazine-2-carbonitrile |
| 249 | 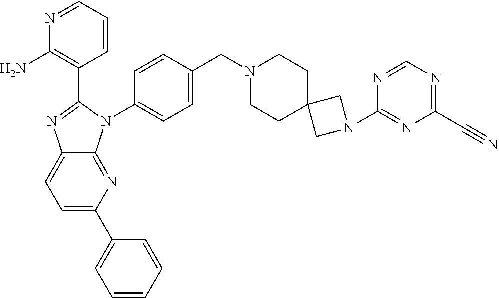 [SMILES: N#Cc1ncnc(N2CC3(CCN(Cc4ccc(-n5c(-c6cccnc6N)nc6ccc(-c7ccccc7)nc65)cc4)CC3)C2)n1] | 4-(7-(4-(2-(2-aminopyridin-3-yl)-5-phenyl- 3H-imidazo[4,5-b]pyridin-3-yl)benzyl)-2,7- diazaspiro[3.5]nonan-2-yl)-1,3,5-triazine-2- carbonitrile |
| 250 | 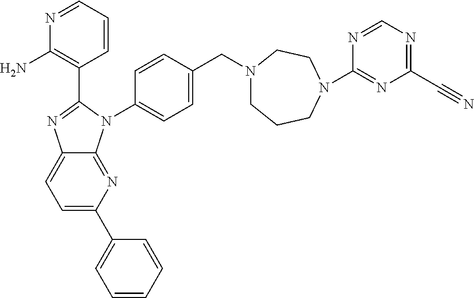 [SMILES: N#Cc1ncnc(N2CCCN(Cc3ccc(-n4c(-c5cccnc5N)nc5ccc(-c6ccccc6)nc54)cc3)CC2)n1] | 4-(4-(4-(2-(2-aminopyridin-3-yl)-5-phenyl- 3H-imidazo[4,5-b]pyridin-3-yl)benzyl)-1,4- diazepan-1-yl)-1,3,5-triazine-2-carbonitrile |
| 251 | 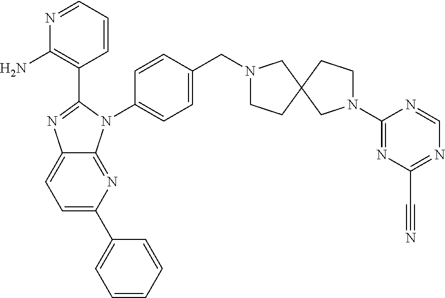 [SMILES: N#Cc1ncnc(N2CCC3(CCN(Cc4ccc(-n5c(-c6cccnc6N)nc6ccc(-c7ccccc7)nc65)cc4)C3)C2)n1] | 4-(7-(4-(2-(2-aminopyridin-3-yl)-5-phenyl- 3H-imidazo[4,5-b]pyridin-3-yl)benzyl)-2,7- diazaspiro[4.4]nonan-2-yl)-1,3,5-triazine-2- carbonitrile |
| 252 | 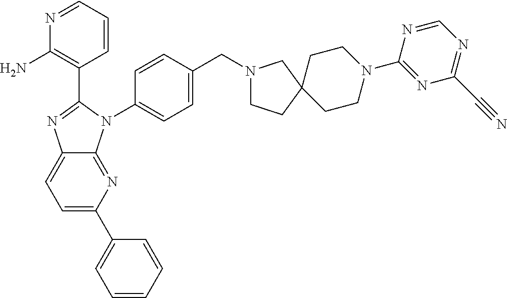 [SMILES: N#Cc1ncnc(N2CCC3(CCN(Cc4ccc(-n5c(-c6cccnc6N)nc6ccc(-c7ccccc7)nc65)cc4)C3)CC2)n1] | 4-(2-(4-(2-(2-aminopyridin-3-yl)-5-phenyl- 3H-imidazo[4,5-b]pyridin-3-yl)benzyl)-2,8- diazaspiro[4.5]decan-8-yl)-1,3,5-triazine-2- carbonitrile |
| 253 | 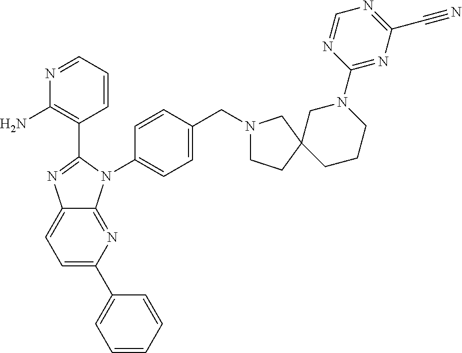 [SMILES: N#Cc1ncnc(N2CCCC3(CCN(Cc4ccc(-n5c(-c6cccnc6N)nc6ccc(-c7ccccc7)nc65)cc4)C3)C2)n1] | 4-(2-(4-(2-(2-aminopyridin-3-yl)-5-phenyl- 3H-imidazo[4,5-b]pyridin-3-yl)benzyl)-2,7- diazaspiro[4.5]decan-7-yl)-1,3,5-triazine-2- carbonitrile |
| 254 | 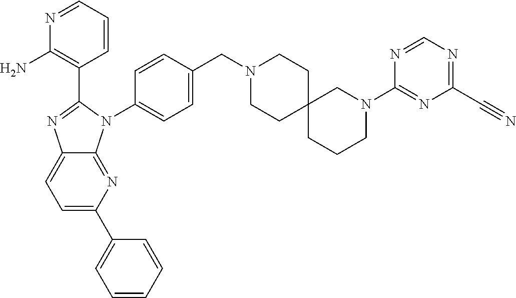 [SMILES: N#Cc1ncnc(N2CCCC3(CCN(Cc4ccc(-n5c(-c6cccnc6N)nc6ccc(-c7ccccc7)nc65)cc4)CC3)C2)n1] | 4-(9-(4-(2-(2-aminopyridin-3-yl)-5-phenyl- 3H-imidazo[4,5-b]pyridin-3-yl)benzyl)-2,9- diazaspiro[5.5]undecan-2-yl)-1,3,5-triazine- 2-carbonitrile |
| 255 | 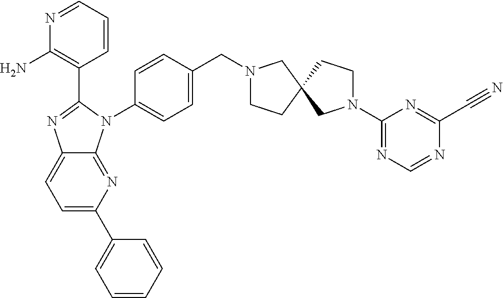 [SMILES: N#Cc1ncnc(N2CC[C@@]3(CCN(Cc4ccc(-n5c(-c6cccnc6N)nc6ccc(-c7ccccc7)nc65)cc4)C3)C2)n1] | (R)-4-(7-(4-(2-(2-aminopyridin-3-yl)-5- phenyl-3H-imidazo[4,5-b]pyridin-3- yl)benzyl)-2,7-diazaspiro[4.4]nonan-2-yl)- 1,3,5-triazine-2-carbonitrile |
| 256 | 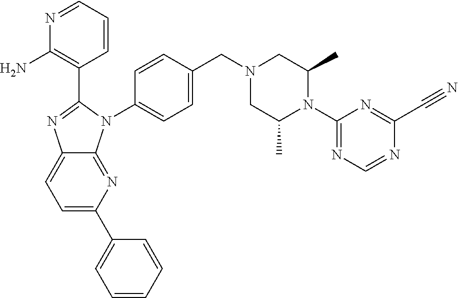 [SMILES: C[C@@H]1CN(Cc2ccc(-n3c(-c4cccnc4N)nc4ccc(-c5ccccc5)nc43)cc2)C[C@@H](C)N1c1ncnc(C#N)n1] | 4-((2R,6R)-4-(4-(2-(2-aminopyridin-3-yl)- 5-phenyl-3H-imidazo[4,5-b]pyridin-3- yl)benzyl)-2,6-dimethylpiperazin-1-yl)- 1,3,5-triazine-2-carbonitrile |
| 257 | 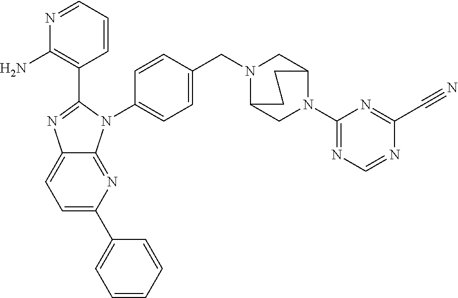 [SMILES: N#Cc1ncnc(N2CC3CCC2CN3Cc2ccc(-n3c(-c4cccnc4N)nc4ccc(-c5ccccc5)nc43)cc2)n1] | 4-(5-(4-(2-(2-aminopyridin-3-yl)-5-phenyl- 3H-imidazo[4,5-b]pyridin-3-yl)benzyl)-2,5- diazabicyclo[2.2.2]octan-2-yl)-1,3,5- triazine-2-carbonitrile |
| 258 | 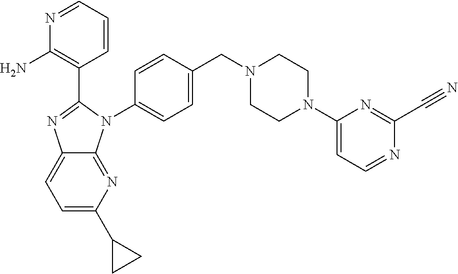 [SMILES: N#Cc1nccc(N2CCN(Cc3ccc(-n4c(-c5cccnc5N)nc5ccc(C6CC6)nc54)cc3)CC2)n1] | 4-(4-(4-(2-(2-aminopyridin-3-yl)-5- cyclopropyl-3H-imidazo[4,5-b]pyridin-3- yl)benzyl)piperazin-1-yl)pyrimidine-2- carbonitrile |
| 259 | 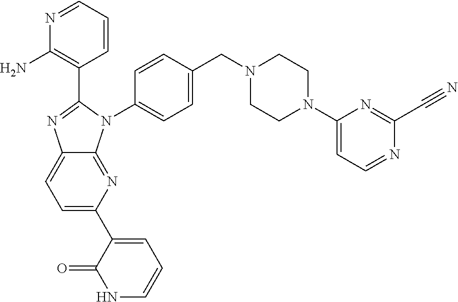 [SMILES: N#Cc1nccc(N2CCN(Cc3ccc(-n4c(-c5cccnc5N)nc5ccc(-c6ccc[nH]c6=O)nc54)cc3)CC2)n1] | 4-(4-(4-(2-(2-aminopyridin-3-yl)-5-(2-oxo- 1,2-dihydropyridin-3-yl)-3H-imidazo[4,5- b]pyridin-3-yl)benzyl)piperazin-1- yl)pyrimidine-2-carbonitrile |
| 260 | 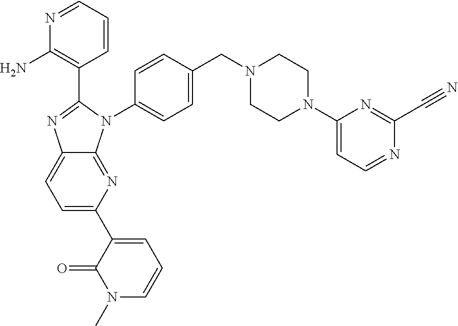 [SMILES: Cn1cccc(-c2ccc3nc(-c4cccnc4N)n(-c4ccc(CN5CCN(c6ccnc(C#N)n6)CC5)cc4)c3n2)c1=O] | 4-(4-(4-(2-(2-aminopyridin-3-yl)-5-(1- methyl-2-oxo-1,2-dihydropyridin-3-yl)-3H- imidazo[4,5-b]pyridin-3- yl)benzyl)piperazin-1-yl)pyrimidine-2- carbonitrile |
| 261 | 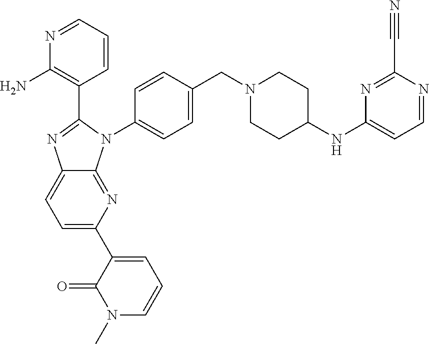 [SMILES: Cn1cccc(-c2ccc3nc(-c4cccnc4N)n(-c4ccc(CN5CCC(Nc6ccnc(C#N)n6)CC5)cc4)c3n2)c1=O] | 4-((1-(4-(2-(2-aminopyridin-3-yl)-5-(1- methyl-2-oxo-1,2-dihydropyridin-3-yl)-3H- imidazo[4,5-b]pyridin-3- yl)benzyl)piperidin-4-yl)amino)pyrimidine- 2-carbonitrile |
| 262 | 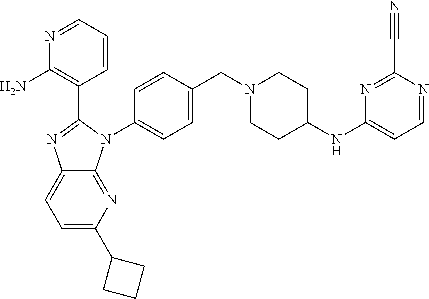 [SMILES: N#Cc1nccc(NC2CCN(Cc3ccc(-n4c(-c5cccnc5N)nc5ccc(C6CCC6)nc54)cc3)CC2)n1] | 4-((1-(4-(2-(2-aminopyridin-3-yl)-5- cyclobutyl-3H-imidazo[4,5-b]pyridin-3- yl)benzyl)piperidin-4-yl)amino)pyrimidine- 2-carbonitrile |
| 263 | 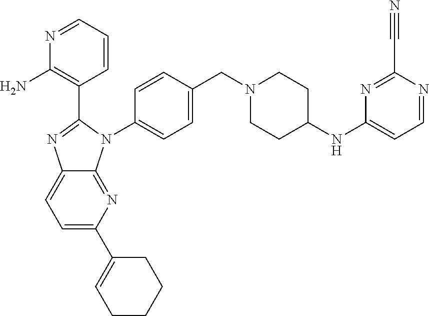 [SMILES: N#Cc1nccc(NC2CCN(Cc3ccc(-n4c(-c5cccnc5N)nc5ccc(C6=CCCCC6)nc54)cc3)CC2)n1] | 4-((1-(4-(2-(2-aminopyridin-3-yl)-5- (cyclohex-1-en-1-yl)-3H-imidazo[4,5- b]pyridin-3-yl)benzyl)piperidin-4- yl)amino)pyrimidine-2-carbonitrile |
| 264 | 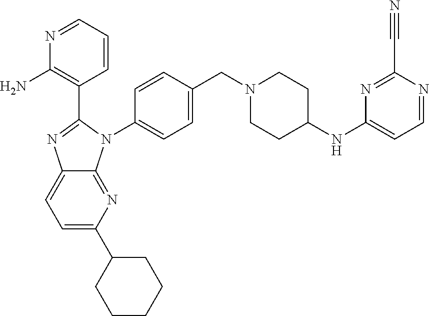 [SMILES: N#Cc1nccc(NC2CCN(Cc3ccc(-n4c(-c5cccnc5N)nc5ccc(C6CCCCC6)nc54)cc3)CC2)n1] | 4-((1-(4-(2-(2-aminopyridin-3-yl)-5- cyclohexyl-3H-imidazo[4,5-b]pyridin-3- yl)benzyl)piperidin-4-yl)amino)pyrimidine- 2-carbonitrile |
| 265 | 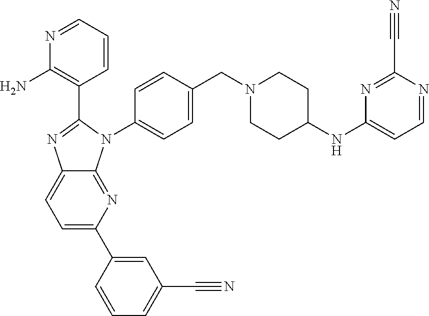 [SMILES: N#Cc1cccc(-c2ccc3nc(-c4cccnc4N)n(-c4ccc(CN5CCC(Nc6ccnc(C#N)n6)CC5)cc4)c3n2)c1] | 4-((1-(4-(2-(2-aminopyridin-3-yl)-5-(3- cyanophenyl)-3H-imidazo[4,5-b]pyridin-3- yl)benzyl)piperidin-4-yl)amino)pyrimidine- 2-carbonitrile |
| 266 | 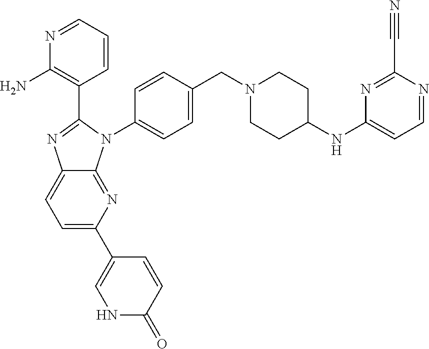 [SMILES: N#Cc1nccc(NC2CCN(Cc3ccc(-n4c(-c5cccnc5N)nc5ccc(-c6ccc(=O)[nH]c6)nc54)cc3)CC2)n1] | 4-((1-(4-(2-(2-aminopyridin-3-yl)-5-(6-oxo- 1,6-dihydropyridin-3-yl)-3H-imidazo[4,5- b]pyridin-3-yl)benzyl)piperidin-4- yl)amino)pyrimidine-2-carbonitrile |
| 267 | 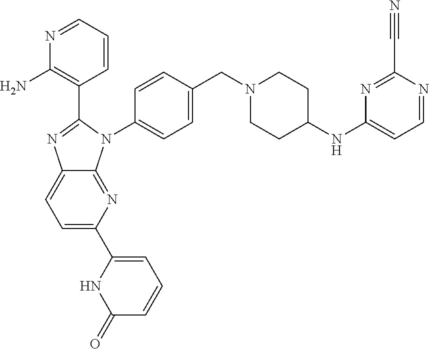 [SMILES: N#Cc1nccc(NC2CCN(Cc3ccc(-n4c(-c5cccnc5N)nc5ccc(-c6cccc(=O)[nH]6)nc54)cc3)CC2)n1] | 4-((1-(4-(2-(2-aminopyridin-3-yl)-5-(6-oxo- 1,6-dihydropyridin-2-yl)-3H-imidazo[4,5- b]pyridin-3-yl)benzyl)piperidin-4- yl)amino)pyrimidine-2-carbonitrile |
| 268 | 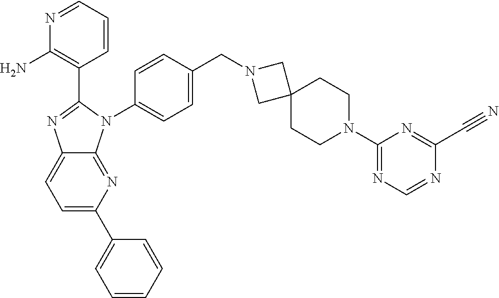 [SMILES: N#Cc1ncnc(N2CCC3(CC2)CN(Cc2ccc(-n4c(-c5cccnc5N)nc5ccc(-c6ccccc6)nc54)cc2)C3)n1] | 4-(2-(4-(2-(2-aminopyridin-3-yl)-5-phenyl- 3H-imidazo[4,5-b]pyridin-3-yl)benzyl)-2,7- diazaspiro[3.5]nonan-7-yl)-1,3,5-triazine-2- carbonitrile |
| 269 | 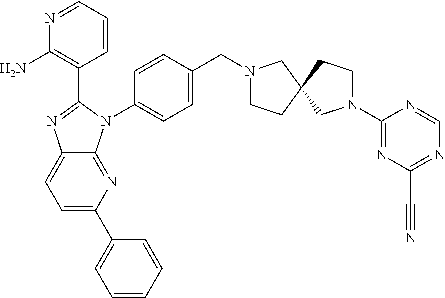 [SMILES: N#Cc1ncnc(N2CC[C@]3(CCN(Cc4ccc(-n5c(-c6cccnc6N)nc6ccc(-c7ccccc7)nc65)cc4)C3)C2)n1] | (S)-4-(7-(4-(2-(2-aminopyridin-3-yl)-5- phenyl-3H-imidazo[4,5-b]pyridin-3- yl)benzyl)-2,7-diazaspiro[4.4]nonan-2-yl)- 1,3,5-triazine-2-carbonitrile |
| 270 | 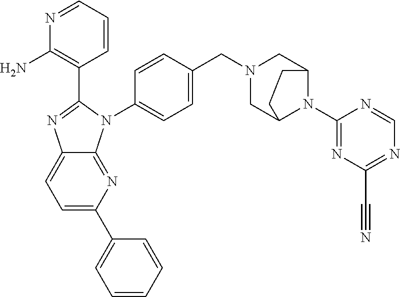 [SMILES: N#Cc1ncnc(N2C3CCC2CN(Cc2ccc(-n4c(-c5cccnc5N)nc5ccc(-c6ccccc6)nc54)cc2)C3)n1] | 4-(3-(4-(2-(2-aminopyridin-3-yl)-5-phenyl- 3H-imidazo[4,5-b]pyridin-3-yl)benzyl)-3,8- diazabicyclo[3.2.1]octan-8-yl)-1,3,5- triazine-2-carbonitrile |
| 271 | 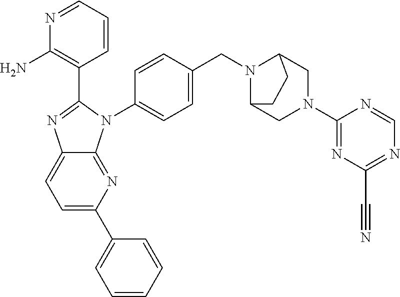 [SMILES: N#Cc1ncnc(N2CC3CCC(C2)N3Cc2ccc(-n3c(-c4cccnc4N)nc4ccc(-c5ccccc5)nc43)cc2)n1] | 4-(8-(4-(2-(2-aminopyridin-3-yl)-5-phenyl- 3H-imidazo[4,5-b]pyridin-3-yl)benzyl)-3,8- diazabicyclo[3.2.1]octan-3-yl)-1,3,5- triazine-2-carbonitrile |
| 272 | 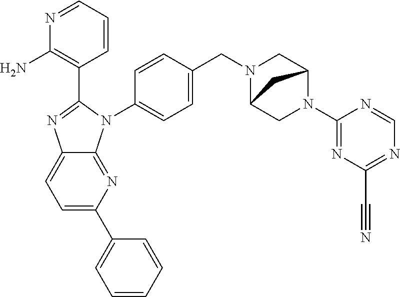 [SMILES: N#Cc1ncnc(N2C[C@H]3C[C@@H]2CN3Cc2ccc(-n3c(-c4cccnc4N)nc4ccc(-c5ccccc5)nc43)cc2)n1] | 4-((1R,4R)-5-(4-(2-(2-aminopyridin-3-yl)- 5-phenyl-3H-imidazo[4,5-b]pyridin-3- yl)benzyl)-2,5-diazabicyclo[2.2.1]heptan-2- yl)-1,3,5-triazine-2-carbonitrile |
| 273 | 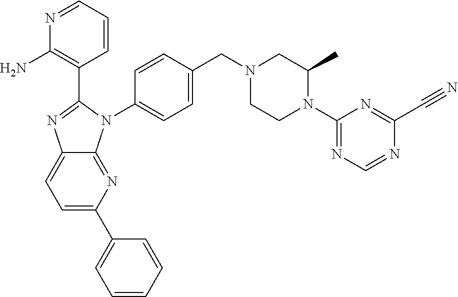 [SMILES: C[C@@H]1CN(Cc2ccc(-n3c(-c4cccnc4N)nc4ccc(-c5ccccc5)nc43)cc2)CCN1c1ncnc(C#N)n1] | (R)-4-(4-(4-(2-(2-aminopyridin- 3-yl)-5- phenyl-3H-imidazo[4,5-b]pyridin-3- yl)benzyl)-2-methylpiperazin-1-yl)-1,3,5- triazine-2-carbonitrile |
| 274 | 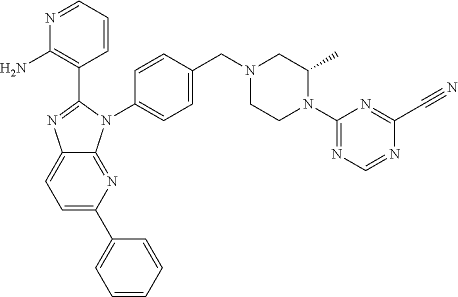 [SMILES: C[C@H]1CN(Cc2ccc(-n3c(-c4cccnc4N)nc4ccc(-c5ccccc5)nc43)cc2)CCN1c1ncnc(C#N)n1] | (S)-4-(4-(4-(2-(2-aminopyridin-3-yl)-5- phenyl-3H-imidazo[4,5-b]pyridin-3- yl)benzyl)-2-methylpiperazin-1-yl)-1,3,5- triazine-2-carbonitrile |
| 275 | 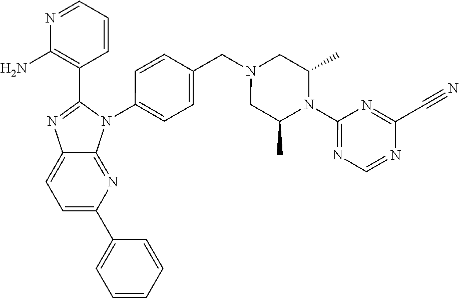 [SMILES: C[C@H]1CN(Cc2ccc(-n3c(-c4cccnc4N)nc4ccc(-c5ccccc5)nc43)cc2)C[C@H](C)N1c1ncnc(C#N)n1] | 4-((2S,6S)-4-(4-(2-(2-aminopyridin-3-yl)-5- phenyl-3H-imidazo[4,5-b]pyridin-3- yl)benzyl)-2,6-dimethylpiperazin-1-yl)- 1,3,5-triazine-2-carbonitrile |
| 276 | 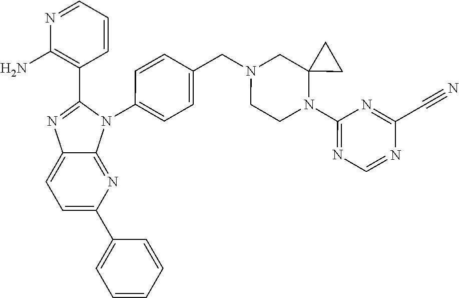 [SMILES: N#Cc1ncnc(N2CCN(Cc3ccc(-n4c(-c5cccnc5N)nc5ccc(-c6ccccc6)nc54)cc3)CC23CC3)n1] | 4-(7-(4-(2-(2-aminopyridin-3-yl)-5-phenyl- 3H-imidazo[4,5-b]pyridin-3-yl)benzyl)-4,7- diazaspiro[2.5]octan-4-yl)-1,3,5-triazine-2- carbonitrile |
| 277 | 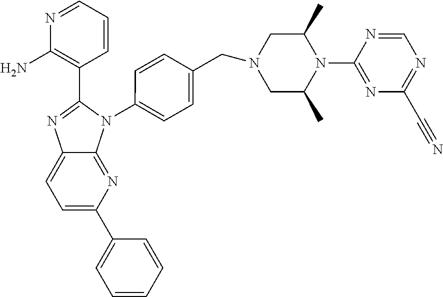 [SMILES: C[C@@H]1CN(Cc2ccc(-n3c(-c4cccnc4N)nc4ccc(-c5ccccc5)nc43)cc2)C[C@H](C)N1c1ncnc(C#N)n1] | 4-((2R,6S)-4-(4-(2-(2-aminopyridin-3-yl)-5- phenyl-3H-imidazo[4,5-b]pyridin-3- yl)benzyl)-2,6-dimethylpiperazin-1-yl)- 1,3,5-triazine-2-carbonitrile |
| 278 | 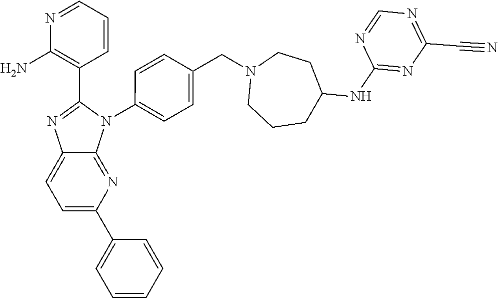 [SMILES: N#Cc1ncnc(NC2CCCN(Cc3ccc(-n4c(-c5cccnc5N)nc5ccc(-c6ccccc6)nc54)cc3)CC2)n1] | 4-((1-(4-(2-(2-aminopyridin-3-yl)-5-phenyl- 3H-imidazo[4,5-b]pyridin-3- yl)benzyl)azepan-4-yl)amino)-1,3,5- triazine-2-carbonitrile |
| 279 | 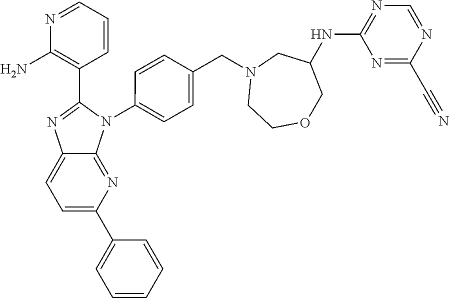 [SMILES: N#Cc1ncnc(NC2COCCN(Cc3ccc(-n4c(-c5cccnc5N)nc5ccc(-c6ccccc6)nc54)cc3)C2)n1] | 4-((4-(4-(2-(2-aminopyridin-3-yl)-5-phenyl- 3H-imidazo[4,5-b]pyridin-3-yl)benzyl)-1,4- oxazepan-6-yl)amino)-1,3,5-triazine-2- carbonitrile |
| 280 | 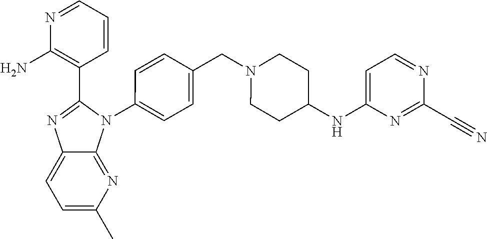 [SMILES: Cc1ccc2nc(-c3cccnc3N)n(-c3ccc(CN4CCC(Nc5ccnc(C#N)n5)CC4)cc3)c2n1] | 4-((1-(4-(2-(2-aminopyridin-3-yl)-5-methyl- 3H-imidazo[4,5-b]pyridin-3- yl)benzyl)piperidin-4-yl)amino)pyrimidine- 2-carbonitrile |
| 281 | 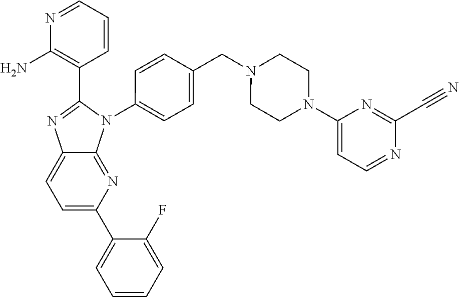 [SMILES: N#Cc1nccc(N2CCN(Cc3ccc(-n4c(-c5cccnc5N)nc5ccc(-c6ccccc6F)nc54)cc3)CC2)n1] | 4-(4-(4-(2-(2-aminopyridin-3-yl)-5-(2- fluorophenyl)-3H-imidazo[4,5-b]pyridin-3- yl)benzyl)piperazin-1-yl)pyrimidine-2- carbonitrile |
| 282 | 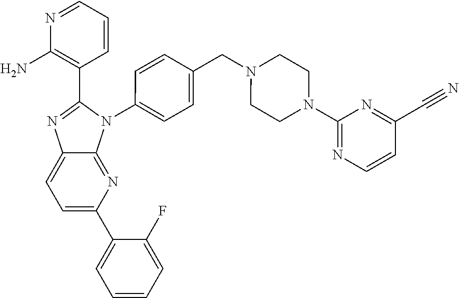 [SMILES: N#Cc1ccnc(N2CCN(Cc3ccc(-n4c(-c5cccnc5N)nc5ccc(-c6ccccc6F)nc54)cc3)CC2)n1] | 2-(4-(4-(2-(2-aminopyridin-3-yl)-5-(2- fluorophenyl)-3H-imidazo[4,5-b]pyridin-3- yl)benzyl)piperazin-1-yl)pyrimidine-4- carbonitrile |
| 283 | 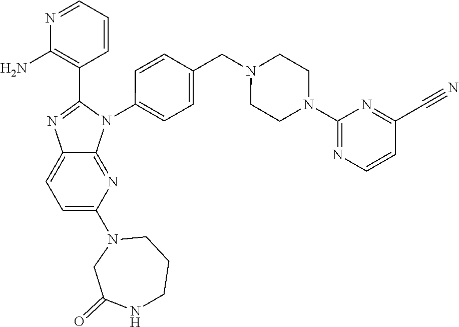 [SMILES: N#Cc1ccnc(N2CCN(Cc3ccc(-n4c(-c5cccnc5N)nc5ccc(N6CCCNC(=O)C6)nc54)cc3)CC2)n1] | 2-(4-(4-(2-(2-aminopyridin-3-yl)-5-(3-oxo- 1,4-diazepan-1-yl)-3H-imidazo[4,5- b]pyridin-3-yl)benzyl)piperazin-1- yl)pyrimidine-4-carbonitrile |
| 284 | 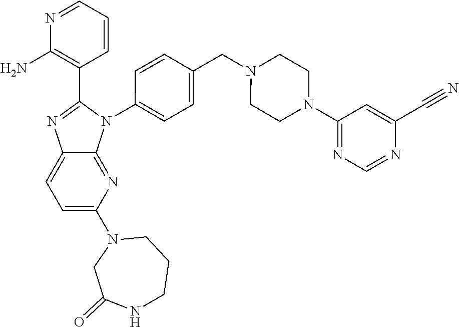 [SMILES: N#Cc1cc(N2CCN(Cc3ccc(-n4c(-c5cccnc5N)nc5ccc(N6CCCNC(=O)C6)nc54)cc3)CC2)ncn1] | 6-(4-(4-(2-(2-aminopyridin-3-yl)-5-(3-oxo- 1,4-diazepan-1-yl)-3H-imidazo[4,5- b]pyridin-3-yl)benzyl)piperazin-1- yl)pyrimidine-4-carbonitrile |
| 285 | 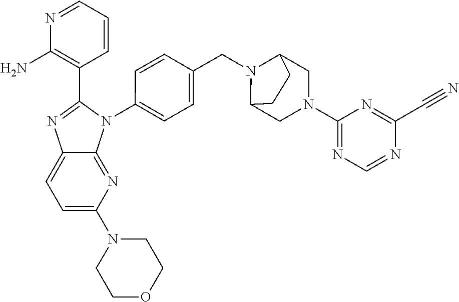 [SMILES: N#Cc1ncnc(N2CC3CCC(C2)N3Cc2ccc(-n3c(-c4cccnc4N)nc4ccc(N5CCOCC5)nc43)cc2)n1] | 4-(8- (4-(2-(2-aminopyridin-3-yl)-5- morpholino-3H-imidazo[4,5-b]pyridin-3- yl)benzyl)-3,8-diazabicyclo[3.2.1]octan-3- yl)-1,3,5-triazine-2-carbonitrile |
| 286 | 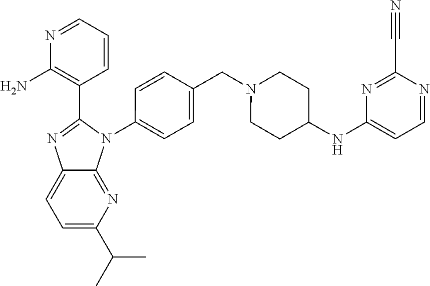 [SMILES: CC(C)c1ccc2nc(-c3cccnc3N)n(-c3ccc(CN4CCC(Nc5ccnc(C#N)n5)CC4)cc3)c2n1] | 4-((1-(4-(2-(2-aminopyridin-3-yl)-5- isopropyl-3H-imidazo[4,5-b]pyridin-3- yl)benzyl)piperidin-4-yl)amino)pyrimidine- 2-carbonitrile |
| 287 | 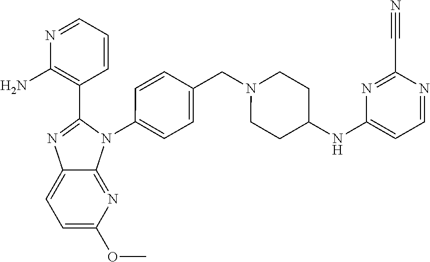 [SMILES: COc1ccc2nc(-c3cccnc3N)n(-c3ccc(CN4CCC(Nc5ccnc(C#N)n5)CC4)cc3)c2n1] | 4-((1-(4-(2-(2-aminopyridin-3-yl)-5- methoxy-3H-imidazo[4,5-b]pyridin-3- yl)benzyl)piperidin-4-yl)amino)pyrimidine- 2-carbonitrile |
| 288 | 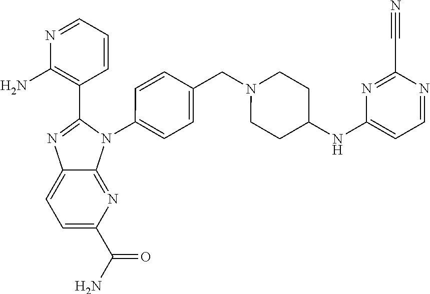 [SMILES: N#Cc1nccc(NC2CCN(Cc3ccc(-n4c(-c5cccnc5N)nc5ccc(C(N)=O)nc54)cc3)CC2)n1] | 2-(2-aminopyridin-3-yl)-3-(4-((4-((2- cyanopyrimidin-4-yl)amino)piperidin-1- yl)methyl)phenyl)-3H-imidazo[4,5- b]pyridine-5-carboxamide |
| 289 | 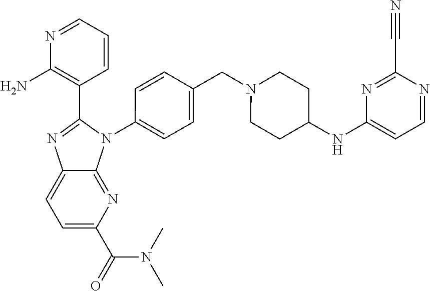 [SMILES: CN(C)C(=O)c1ccc2nc(-c3cccnc3N)n(-c3ccc(CN4CCC(Nc5ccnc(C#N)n5)CC4)cc3)c2n1] | 2-(2-aminopyridin-3-yl)-3-(4-((4-((2- cyanopyrimidin-4-yl)amino)piperidin-1- yl)methyl)phenyl)-N,N-dimethyl-3H- imidazo[4,5-b]pyridine-5-carboxamide |
| 290 | 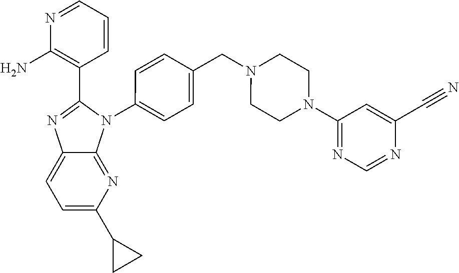 [SMILES: N#Cc1cc(N2CCN(Cc3ccc(-n4c(-c5cccnc5N)nc5ccc(C6CC6)nc54)cc3)CC2)ncn1] | 6-(4-(4-(2-(2-aminopyridin-3-yl)-5- cyclopropyl-3H-imidazo[4,5-b]pyridin-3- yl)benzyl)piperazin-1-yl)pyrimidine-4- carbonitrile |
| 291 | 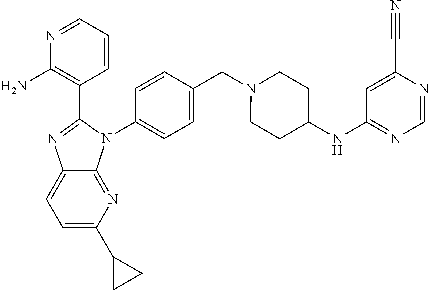 [SMILES: N#Cc1cc(NC2CCN(Cc3ccc(-n4c(-c5cccnc5N)nc5ccc(C6CC6)nc54)cc3)CC2)ncn1] | 6-((1-(4-(2-(2-aminopyridin-3-yl)-5- cyclopropyl-3H-imidazo[4,5-b]pyridin-3- yl)benzyl)piperidin-4-yl)amino)pyrimidine- 4-carbonitrile |
| 292 | 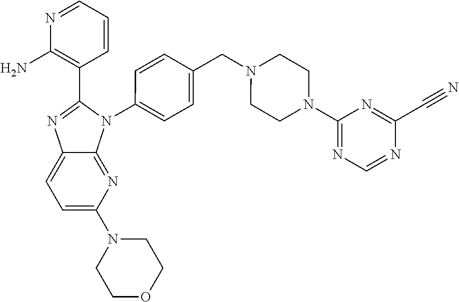 [SMILES: N#Cc1ncnc(N2CCN(Cc3ccc(-n4c(-c5cccnc5N)nc5ccc(N6CCOCC6)nc54)cc3)CC2)n1] | 4-(4-(4-(2-(2-aminopyridin-3-yl)-5- morpholino-3H-imidazo[4,5-b]pyridin-3- yl)benzyl)piperazin-1-yl)-1,3,5-triazine-2- carbonitrile |
| 293 | 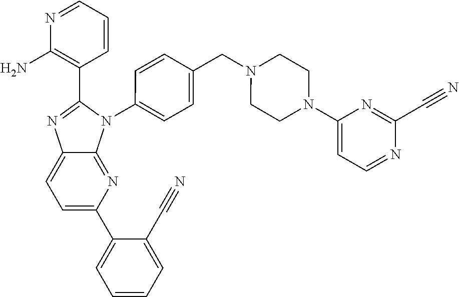 [SMILES: N#Cc1nccc(N2CCN(Cc3ccc(-n4c(-c5cccnc5N)nc5ccc(-c6ccccc6C#N)nc54)cc3)CC2)n1] | 4-(4-(4-(2-(2-aminopyridin-3-yl)-5-(2- cyanophenyl)-3H-imidazo[4,5-b]pyridin-3- yl)benzyl)piperazin-1-yl)pyrimidine-2- carbonitrile |
| 294 | 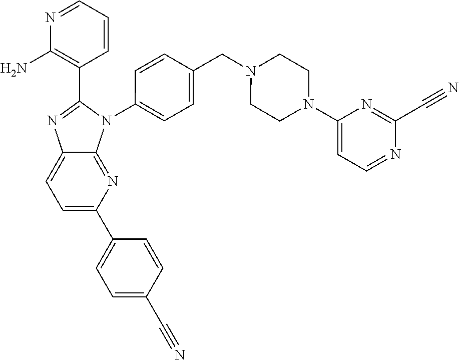 [SMILES: N#Cc1ccc(-c2ccc3nc(-c4cccnc4N)n(-c4ccc(CN5CCN(c6ccnc(C#N)n6)CC5)cc4)c3n2)cc1] | 4-(4-(4-(2-(2-aminopyridin-3-yl)-5-(4- cyanophenyl)-3H-imidazo[4,5-b]pyridin-3- yl)benzyl)piperazin-1-yl)pyrimidine-2- carbonitrile |
| 295 | 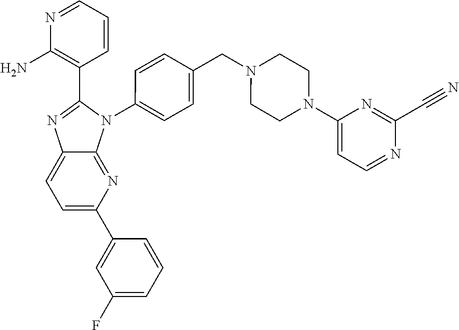 [SMILES: N#Cc1nccc(N2CCN(Cc3ccc(-n4c(-c5cccnc5N)nc5ccc(-c6cccc(F)c6)nc54)cc3)CC2)n1] | 4-(4-(4-(2-(2-aminopyridin-3-yl)-5-(3- fluorophenyl)-3H-imidazo[4,5-b]pyridin-3- yl)benzyl)piperazin-1-yl)pyrimidine-2- carbonitrile |
| 296 | 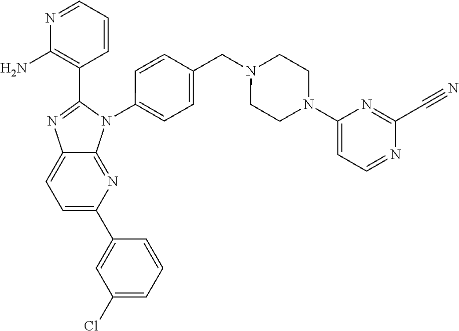 [SMILES: N#Cc1nccc(N2CCN(Cc3ccc(-n4c(-c5cccnc5N)nc5ccc(-c6cccc(Cl)c6)nc54)cc3)CC2)n1] | 4-(4-(4-(2-(2-aminopyridin-3-yl)-5-(3- chlorophenyl)-3H-imidazo[4,5-b]pyridin-3- yl)benzyl)piperazin-1-yl)pyrimidine-2- carbonitrile |
| 297 | 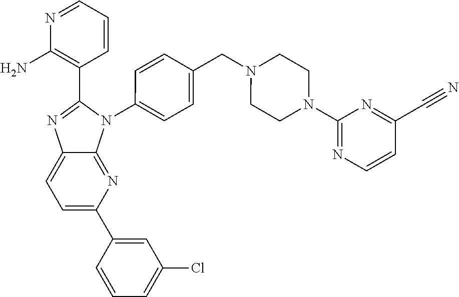 [SMILES: N#Cc1ccnc(N2CCN(Cc3ccc(-n4c(-c5cccnc5N)nc5ccc(-c6cccc(Cl)c6)nc54)cc3)CC2)n1] | 2-(4-(4-(2-(2-aminopyridin-3-yl)-5-(3- chlorophenyl)-3H-imidazo[4,5-b]pyridin-3- yl)benzyl)piperazin-1-yl)pyrimidine-4- carbonitrile |
| 298 | 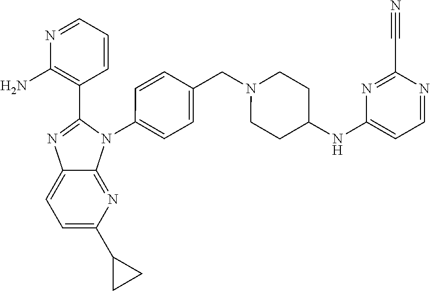 [SMILES: N#Cc1nccc(NC2CCN(Cc3ccc(-n4c(-c5cccnc5N)nc5ccc(C6CC6)nc54)cc3)CC2)n1] | 4-((1-(4-(2-(2-aminopyridin-3-yl)-5- cyclopropyl-3H-imidazo[4,5-b]pyridin-3- yl)benzyl)piperidin-4-yl)amino)pyrimidine- 2-carbonitrile |
| 299 | 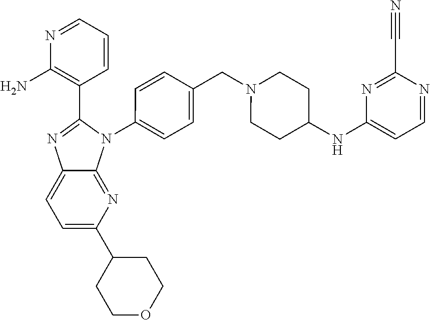 [SMILES: N#Cc1nccc(NC2CCN(Cc3ccc(-n4c(-c5cccnc5N)nc5ccc(C6CCOCC6)nc54)cc3)CC2)n1] | 4-((1-(4-(2-(2-aminopyridin-3-yl)-5- (tetrahydro-2H-pyran-4-yl)-3H- imidazo[4,5-b]pyridin-3- yl)benzyl)piperidin-4-yl)amino)pyrimidine- 2-carbonitrile |
| 300 | 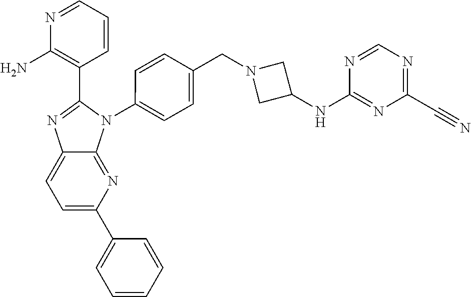 [SMILES: N#Cc1ncnc(NC2CN(Cc3ccc(-n4c(-c5cccnc5N)nc5ccc(-c6ccccc6)nc54)cc3)C2)n1] | 4-((1-(4-(2-(2-aminopyridin-3-yl)-5-phenyl- 3H-imidazo[4,5-b ]pyridin-3- yl)benzyl)azetidin-3-yl)amino)-1,3,5- triazine-2-carbonitrile |
| 301 | 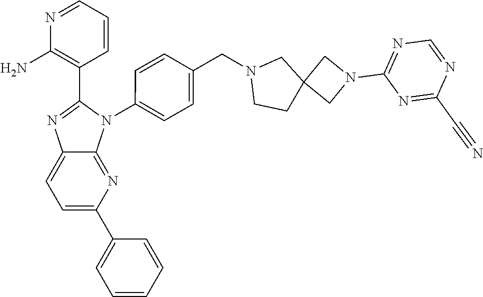 [SMILES: N#Cc1ncnc(N2CC3(CCN(Cc4ccc(-n5c(-c6cccnc6N)nc6ccc(-c7ccccc7)nc65)cc4)C3)C2)n1] | 4-(6-(4-(2-(2-aminopyridin-3-yl)-5-phenyl- 3H-imidazo[4,5-b]pyridin-3-yl)benzyl)-2,6- diazaspiro[3.4]octan-2-yl)-1,3,5-triazine-2- carbonitrile |
| 302 | 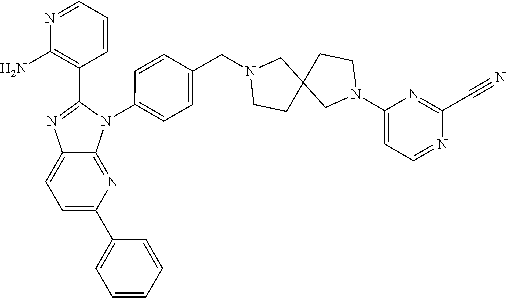 [SMILES: N#Cc1nccc(N2CCC3(CCN(Cc4ccc(-n5c(-c6cccnc6N)nc6ccc(-c7ccccc7)nc65)cc4)C3)C2)n1] | 4-(7-(4-(2-(2-aminopyridin-3-yl)-5-phenyl- 3H-imidazo[4,5-b]pyridin-3-yl)benzyl)-2,7- diazaspiro[4.4]nonan-2-yl)pyrimidine-2- carbonitrile |
| 303 | 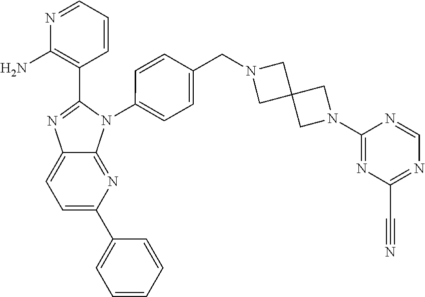 [SMILES: N#Cc1ncnc(N2CC3(CN(Cc4ccc(-n5c(-c6cccnc6N)nc6ccc(-c7ccccc7)nc65)cc4)C3)C2)n1] | 4-(6-(4-(2-(2-aminopyridin-3-yl)-5-phenyl- 3H-imidazo[4,5-b]pyridin-3-yl)benzyl)-2,6- diazaspiro[3.3 ]heptan-2-yl)-1,3,5-triazine-2- carbonitrile |
| 304 | 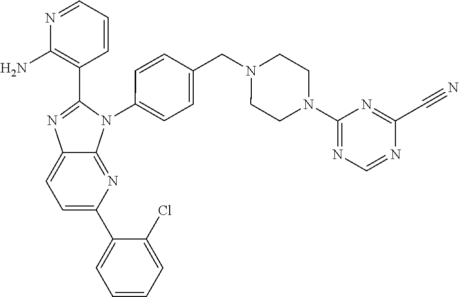 [SMILES: N#Cc1ncnc(N2CCN(Cc3ccc(-n4c(-c5cccnc5N)nc5ccc(-c6ccccc6Cl)nc54)cc3)CC2)n1] | 4-(4-(4-(2-(2-aminopyridin-3-yl)-5-(2- chlorophenyl)-3H-imidazo[4,5-b]pyridin-3- yl)benzyl)piperazin-1-yl)-1,3,5-triazine-2- carbonitrile |
| 305 | 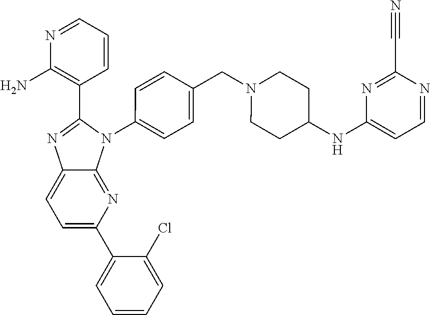 [SMILES: N#Cc1nccc(NC2CCN(Cc3ccc(-n4c(-c5cccnc5N)nc5ccc(-c6ccccc6Cl)nc54)cc3)CC2)n1] | 4-((1-(4-(2-(2-aminopyridin-3-yl)-5-(2- chlorophenyl)-3H-imidazo[4,5-b]pyridin-3- yl)benzyl)piperidin-4-yl)amino)pyrimidine- 2-carbonitrile |
| 306 | 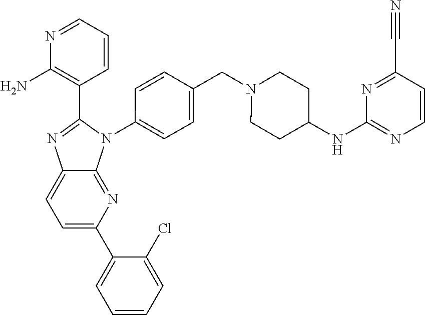 [SMILES: N#Cc1ccnc(NC2CCN(Cc3ccc(-n4c(-c5cccnc5N)nc5ccc(-c6ccccc6Cl)nc54)cc3)CC2)n1] | 2-((1-(4-(2-(2-aminopyridin-3-yl)-5-(2- chlorophenyl)-3H-imidazo[4,5-b]pyridin-3- yl)benzyl)piperidin-4-yl)amino)pyrimidine- 4-carbonitrile |
| 307 | 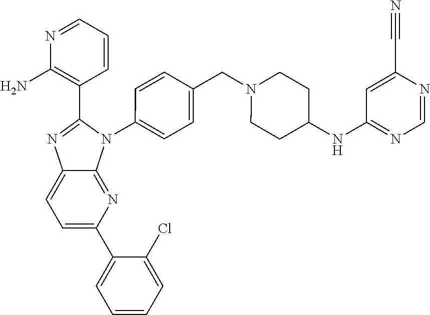 [SMILES: N#Cc1cc(NC2CCN(Cc3ccc(-n4c(-c5cccnc5N)nc5ccc(-c6ccccc6Cl)nc54)cc3)CC2)ncn1] | 6-((1-(4-(2-(2-aminopyridin-3-yl)-5-(2- chlorophenyl)-3H-imidazo[4,5-b]pyridin-3- yl)benzyl)piperidin-4-yl)amino)pyrimidine- 4-carbonitrile |
| 308 | 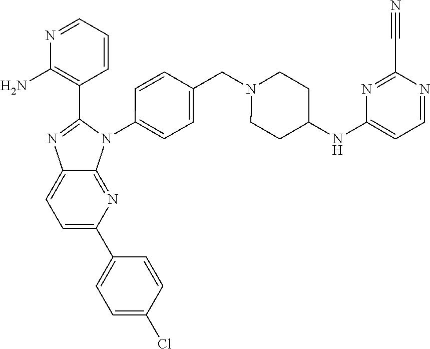 [SMILES: N#Cc1nccc(NC2CCN(Cc3ccc(-n4c(-c5cccnc5N)nc5ccc(-c6ccc(Cl)cc6)nc54)cc3)CC2)n1] | 4-((1-(4-(2-(2-aminopyridin-3-yl)-5-(4- chlorophenyl)-3H-imidazo[4,5-b]pyridin-3- yl)benzyl)piperidin-4-yl)amino)pyrimidine- 2-carbonitrile |
| 309 | 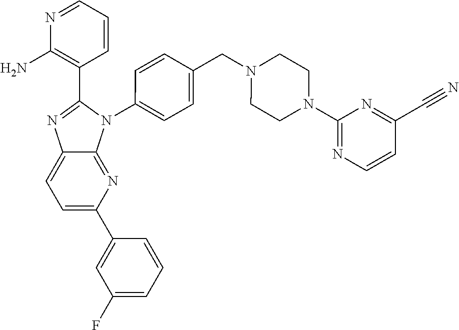 [SMILES: N#Cc1ccnc(N2CCN(Cc3ccc(-n4c(-c5cccnc5N)nc5ccc(-c6cccc(F)c6)nc54)cc3)CC2)n1] | 2-(4-(4-(2-(2-aminopyridin-3-yl)-5-(3- fluorophenyl)-3H-imidazo[4,5-b]pyridin-3- yl)benzyl)piperazin-1-yl)pyrimidine-4- carbonitrile |
| 310 | 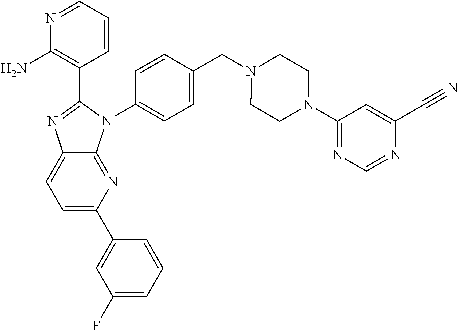 [SMILES: N#Cc1cc(N2CCN(Cc3ccc(-n4c(-c5cccnc5N)nc5ccc(-c6cccc(F)c6)nc54)cc3)CC2)ncn1] | 6-(4-(4-(2-(2-aminopyridin-3-yl)-5-(3- fluorophenyl)-3H-imidazo[4,5-b]pyridin-3- yl)benzyl)piperazin-1-yl)pyrimidine-4- carbonitrile |
| 311 | 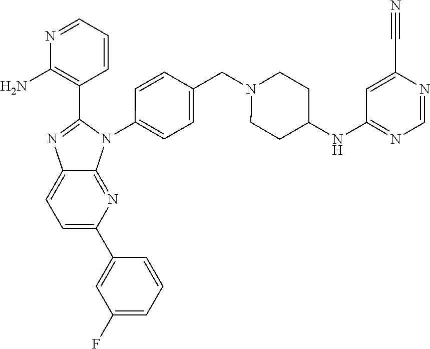 [SMILES: N#Cc1cc(NC2CCN(Cc3ccc(-n4c(-c5cccnc5N)nc5ccc(-c6cccc(F)c6)nc54)cc3)CC2)ncn1] | 6-((1-(4-(2-(2-aminopyridin-3-yl)-5-(3- fluorophenyl)-3H-imidazo[4,5-b]pyridin-3- yl)benzyl)piperidin-4-yl)amino)pyrimidine- 4-carbonitrile |
| 312 | 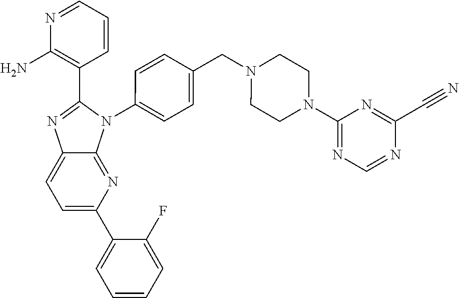 [SMILES: N#Cc1ncnc(N2CCN(Cc3ccc(-n4c(-c5cccnc5N)nc5ccc(-c6ccccc6F)nc54)cc3)CC2)n1] | 4-(4-(4-(2-(2-aminopyridin-3-yl)-5-(2- fluorophenyl)-3H-imidazo[4,5-b]pyridin-3- yl)benzyl)piperazin-1-yl)-1,3,5-triazine-2- carbonitrile |
| 313 | 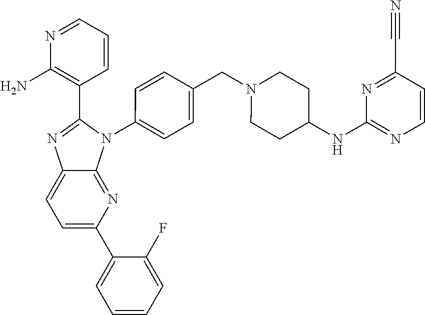 [SMILES: N#Cc1ccnc(NC2CCN(Cc3ccc(-n4c(-c5cccnc5N)nc5ccc(-c6ccccc6F)nc54)cc3)CC2)n1] | 2-((1-(4-(2-(2-aminopyridin-3-yl)-5-(2- fluorophenyl)-3H-imidazo[4,5-b]pyridin-3- yl)benzyl)piperidin-4-yl)amino)pyrimidine- 4-carbonitrile |
| 314 | 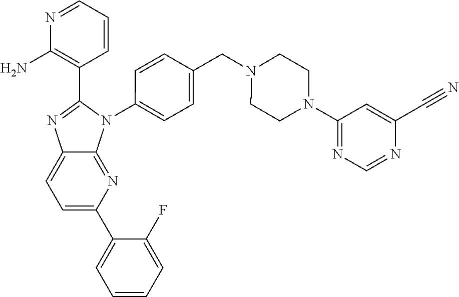 [SMILES: N#Cc1cc(N2CCN(Cc3ccc(-n4c(-c5cccnc5N)nc5ccc(-c6ccccc6F)nc54)cc3)CC2)ncn1] | 6-(4-(4-(2-(2-aminopyridin-3-yl)-5-(2- fluorophenyl)-3H-imidazo[4,5-b]pyridin-3- yl)benzyl)piperazin-1-yl)pyrimidine-4- carbonitrile |
| 315 | 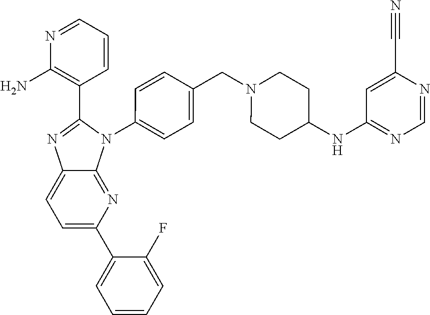 [SMILES: N#Cc1cc(NC2CCN(Cc3ccc(-n4c(-c5cccnc5N)nc5ccc(-c6ccccc6F)nc54)cc3)CC2)ncn1] | 6-((1-(4-(2-(2-aminopyridin-3-yl)-5-(2- fluorophenyl)-3H-imidazo[4,5-b]pyridin-3- yl)benzyl)piperidin-4-yl)amino)pyrimidine- 4-carbonitrile |
| 316 | 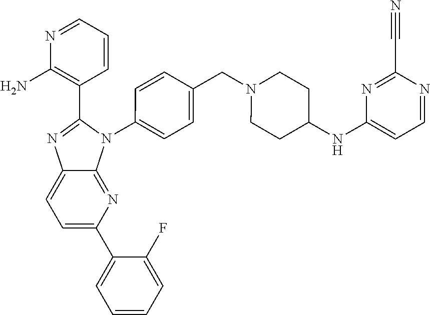 [SMILES: N#Cc1nccc(NC2CCN(Cc3ccc(-n4c(-c5cccnc5N)nc5ccc(-c6ccccc6F)nc54)cc3)CC2)n1] | 4-((1-(4-(2-(2-aminopyridin-3-yl)-5-(2- fluorophenyl)-3H-imidazo[4,5-b]pyridin-3- yl)benzyl)piperidin-4-yl)amino)pyrimidine- 2-carbonitrile |
| 317 | 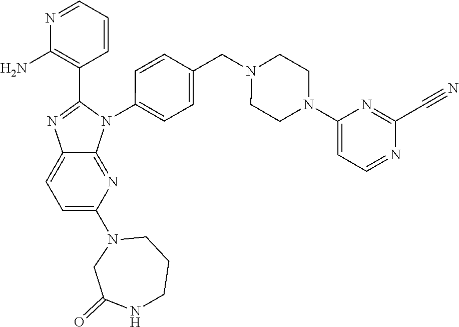 [SMILES: N#Cc1nccc(N2CCN(Cc3ccc(-n4c(-c5cccnc5N)nc5ccc(N6CCCNC(=O)C6)nc54)cc3)CC2)n1] | 4-(4-(4-(2-(2-aminopyridin-3-yl)-5-(3-oxo- 1,4-diazepan-1-yl)-3H-imidazo[4,5- b]pyridin-3-yl)benzyl)piperazin-1- yl)pyrimidine-2-carbonitrile |
| 318 | 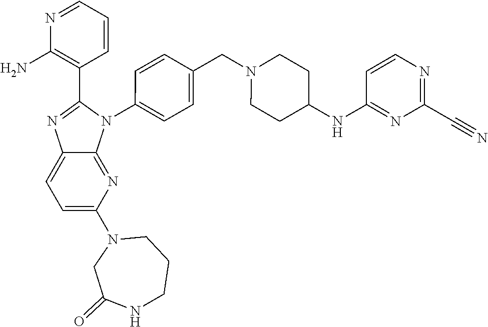 [SMILES: N#Cc1nccc(NC2CCN(Cc3ccc(-n4c(-c5cccnc5N)nc5ccc(N6CCCNC(=O)C6)nc54)cc3)CC2)n1] | 4-((1-(4-(2-(2-aminopyridin-3-yl)-5-(3-oxo- 1,4-diazepan-1-yl)-3H-imidazo[4,5- b]pyridin-3-yl)benzyl)piperidin-4- yl)amino)pyrimidine-2-carbonitrile |
| 319 | 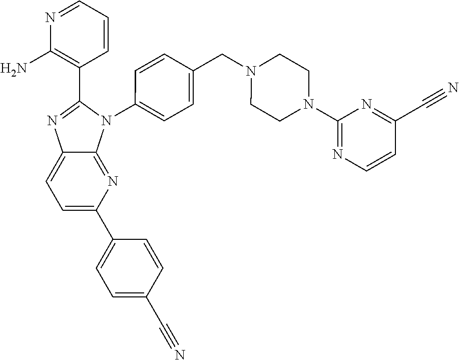 [SMILES: N#Cc1ccc(-c2ccc3nc(-c4cccnc4N)n(-c4ccc(CN5CCN(c6nccc(C#N)n6)CC5)cc4)c3n2)cc1] | 2-(4-(4-(2-(2-aminopyridin-3-yl)-5-(4- cyanophenyl)-3H-imidazo[4,5-b]pyridin-3- yl)benzyl)piperazin-1-yl)pyrimidine-4- carbonitrile |
| 320 | 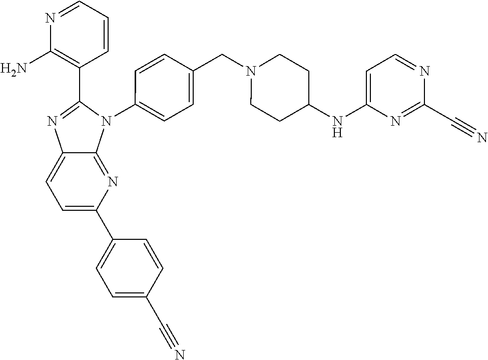 [SMILES: N#Cc1ccc(-c2ccc3nc(-c4cccnc4N)n(-c4ccc(CN5CCC(Nc6ccnc(C#N)n6)CC5)cc4)c3n2)cc1] | 4-((1-(4-(2-(2-aminopyridin-3-yl)-5-(4- cyanophenyl)-3H-imidazo[4,5-b]pyridin-3- yl)benzyl)piperidin-4-yl)amino)pyrimidine- 2-carbonitrile |
| 321 | 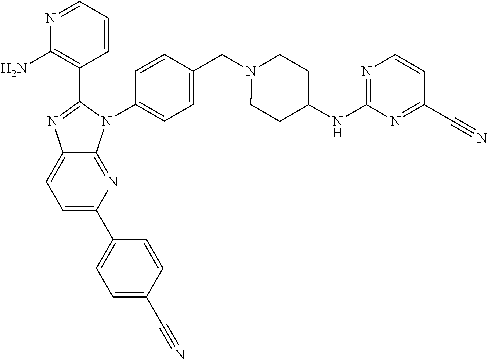 [SMILES: N#Cc1ccc(-c2ccc3nc(-c4cccnc4N)n(-c4ccc(CN5CCC(Nc6nccc(C#N)n6)CC5)cc4)c3n2)cc1] | 2-((1-(4-(2-(2-aminopyridin-3-yl)-5-(4- cyanophenyl)-3H-imidazo[4,5-b]pyridin-3- yl)benzyl)piperidin-4-yl)amino)pyrimidine- 4-carbonitrile |
| 322 | 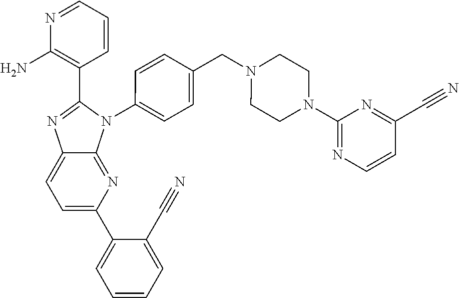 [SMILES: N#Cc1ccnc(N2CCN(Cc3ccc(-n4c(-c5cccnc5N)nc5ccc(-c6ccccc6C#N)nc54)cc3)CC2)n1] | 2-(4-(4-(2-(2-aminopyridin-3-yl)-5-(2- cyanophenyl)-3H-imidazo[4,5-b]pyridin-3- yl)benzyl)piperazin-1-yl)pyrimidine-4- carbonitrile |
| 323 | 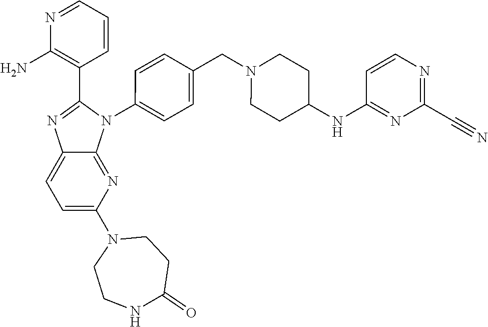 [SMILES: N#Cc1nccc(NC2CCN(Cc3ccc(-n4c(-c5cccnc5N)nc5ccc(N6CCNC(=O)CC6)nc54)cc3)CC2)n1] | 4-((1-(4-(2-(2-aminopyridin-3-yl)-5-(5-oxo- 1,4-diazepan-1-yl)-3H-imidazo[4,5- b]pyridin-3-yl)benzyl)piperidin-4- yl)amino)pyrimidine-2-carbonitrile |
| 324 | 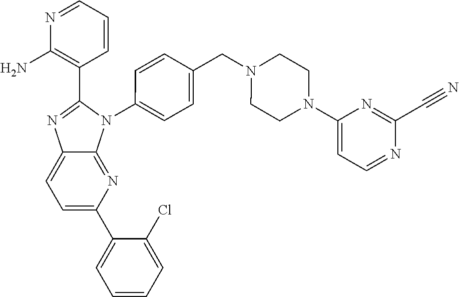 [SMILES: N#Cc1nccc(N2CCN(Cc3ccc(-n4c(-c5cccnc5N)nc5ccc(-c6ccccc6Cl)nc54)cc3)CC2)n1] | 4-(4-(4-(2-(2-aminopyridin-3-yl)-5-(2- chlorophenyl)-3H-imidazo[4,5-b]pyridin-3- yl)benzyl)piperazin-1-yl)pyrimidine-2- carbonitrile |
| 325 | 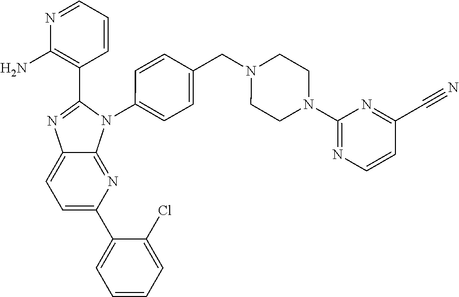 [SMILES: N#Cc1ccnc(N2CCN(Cc3ccc(-n4c(-c5cccnc5N)nc5ccc(-c6ccccc6Cl)nc54)cc3)CC2)n1] | 2-(4-(4-(2-(2-aminopyridin-3-yl)-5-(2- chlorophenyl)-3H-imidazo[4,5-b]pyridin-3- yl)benzyl)piperazin-1-yl)pyrimidine-4- carbonitrile |
| 326 | 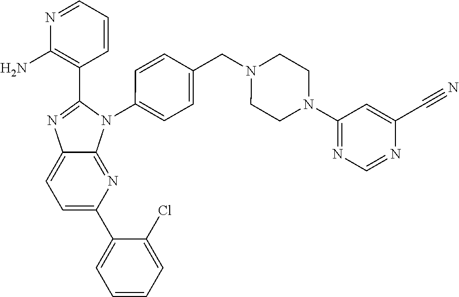 [SMILES: N#Cc1cc(N2CCN(Cc3ccc(-n4c(-c5cccnc5N)nc5ccc(-c6ccccc6Cl)nc54)cc3)CC2)ncn1] | 6-(4-(4-(2-(2-aminopyridin-3-yl)-5-(2- chlorophenyl)-3H-imidazo[4,5-b]pyridin-3- yl)benzyl)piperazin-1-yl)pyrimidine-4- carbonitrile |
| 327 | 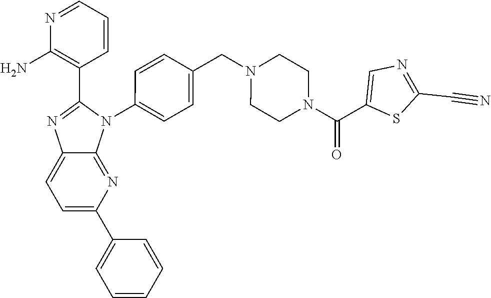 [SMILES: N#Cc1ncc(C(=O)N2CCN(Cc3ccc(-n4c(-c5cccnc5N)nc5ccc(-c6ccccc6)nc54)cc3)CC2)s1] | 5-(4-(4-(2-(2-aminopyridin-3-yl)-5-phenyl- 3H-imidazo[4,5-b]pyridin-3- yl)benzyl)piperazine-1-carbonyl)thiazole-2- carbonitrile |
| 328 | 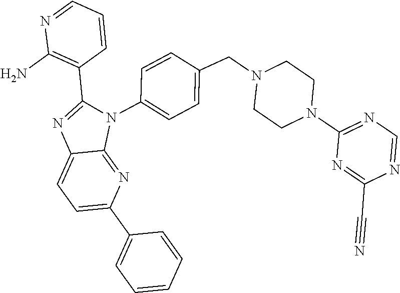 [SMILES: N#Cc1ncnc(N2CCN(Cc3ccc(-n4c(-c5cccnc5N)nc5ccc(-c6ccccc6)nc54)cc3)CC2)n1] | 4-(4-(4-(2-(2-aminopyridin-3-yl)-5-phenyl- 3H-imidazo[4,5-b]pyridin-3- yl)benzyl)piperazin-1-yl)-1,3,5-triazine-2- carbonitrile |
| 329 | 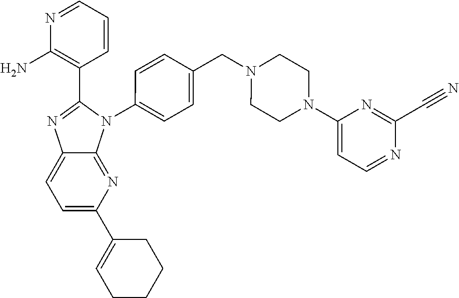 [SMILES: N#Cc1nccc(N2CCN(Cc3ccc(-n4c(-c5cccnc5N)nc5ccc(C6=CCCCC6)nc54)cc3)CC2)n1] | 4-(4-(4-(2-(2-aminopyridin-3-yl)-5- (cyclohex-1-en-1-yl)-3H-imidazo[4,5- b]pyridin-3-yl)benzyl)piperazin-1- yl)pyrimidine-2-carbonitrile |
| 330 | 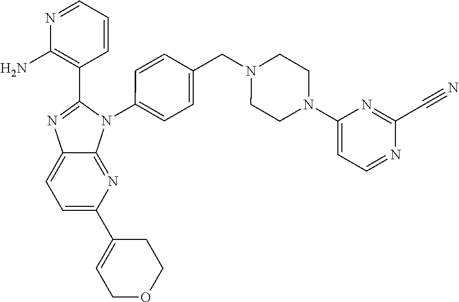 [SMILES: N#Cc1nccc(N2CCN(Cc3ccc(-n4c(-c5cccnc5N)nc5ccc(C6=CCOCC6)nc54)cc3)CC2)n1] | 4-(4-(4-(2-(2-aminopyridin-3-yl)-5-(3,6- dihydro-2H-pyran-4-yl)-3H-imidazo[4,5- b]pyridin-3-yl)benzyl)piperazin-1- yl)pyrimidine-2-carbonitrile |
| 331 | 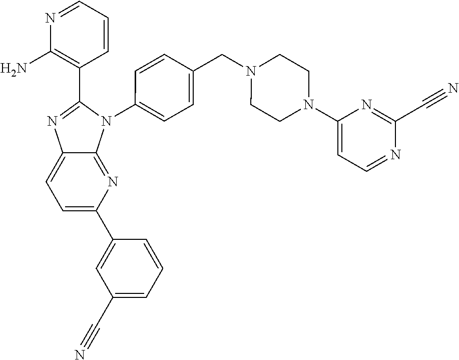 [SMILES: N#Cc1cccc(-c2ccc3nc(-c4cccnc4N)n(-c4ccc(CN5CCN(c6ccnc(C#N)n6)CC5)cc4)c3n2)c1] | 4-(4-(4-(2-(2-aminopyridin-3-yl)-5-(3- cyanophenyl)-3H-imidazo[4,5-b]pyridin-3- yl)benzyl)piperazin-1-yl)pyrimidine-2- carbonitrile |
| 332 | 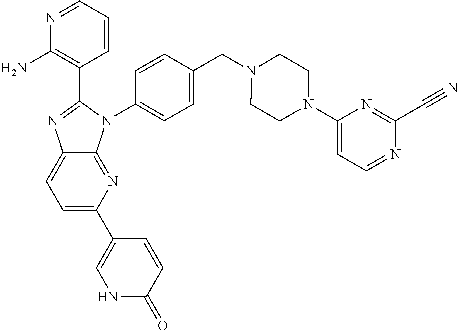 [SMILES: N#Cc1nccc(N2CCN(Cc3ccc(-n4c(-c5cccnc5N)nc5ccc(-c6ccc(=O)[nH]c6)nc54)cc3)CC2)n1] | 4-(4-(4-(2-(2-aminopyridin-3-yl)-5-(6-oxo- 1,6-dihydropyridin-3-yl)-3H-imidazo[4,5- b]pyridin-3-yl)benzyl)piperazin-1- yl)pyrimidine-2-carbonitrile |
| 333 | 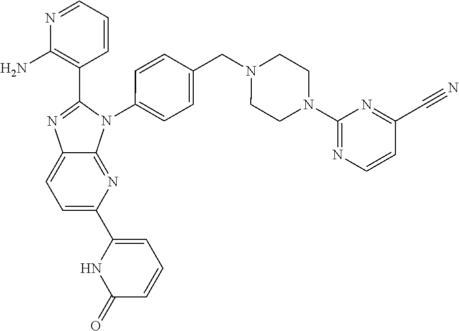 [SMILES: N#Cc1ccnc(N2CCN(Cc3ccc(-n4c(-c5cccnc5N)nc5ccc(-c6cccc(=O)[nH]6)nc54)cc3)CC2)n1] | 2-(4-(4-(2-(2-aminopyridin-3-yl)-5-(6-oxo- 1,6-dihydropyridin-2-yl)-3H-imidazo[4,5- b]pyridin-3-yl)benzyl)piperazin-1- yl)pyrimidine-4-carbonitrile |
| 334 | 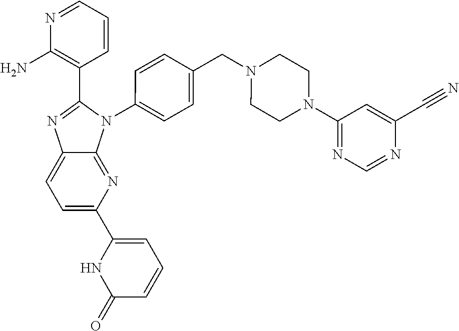 [SMILES: N#Cc1cc(N2CCN(Cc3ccc(-n4c(-c5cccnc5N)nc5ccc(-c6cccc(=O)[nH]6)nc54)cc3)CC2)ncn1] | 6-(4-(4-(2-(2-aminopyridin-3-yl)-5-(6-oxo- 1,6-dihydropyridin-2-yl)-3H-imidazo[4,5- b]pyridin-3-yl)benzyl)piperazin-1- yl)pyrimidine-4-carbonitrile |
| 335 | 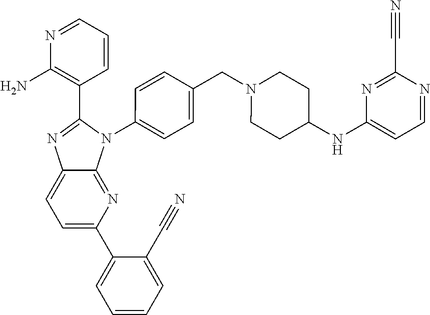 [SMILES: N#Cc1nccc(NC2CCN(Cc3ccc(-n4c(-c5cccnc5N)nc5ccc(-c6ccccc6C#N)nc54)cc3)CC2)n1] | 4-((1-(4-(2-(2-aminopyridin-3-yl)-5-(2- cyanophenyl)-3H-imidazo[4,5-b]pyridin-3- yl)benzyl)piperidin-4-yl)amino)pyrimidine- 2-carbonitrile |
| 336 | 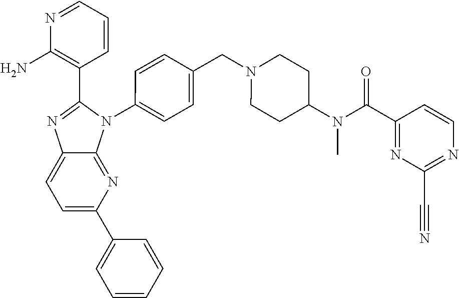 [SMILES: CN(C(=O)c1ccnc(C#N)n1)C1CCN(Cc2ccc(-n3c(-c4cccnc4N)nc4ccc(-c5ccccc5)nc43)cc2)CC1] | N-(1-(4-(2-(2-aminopyridin-3-yl)-5-phenyl- 3H-imidazo[4,5-b ]pyridin-3- yl)benzyl)piperidin-4-yl)-2-cyano-N- methylpyrimidine-4-carboxamide |
| 337 | 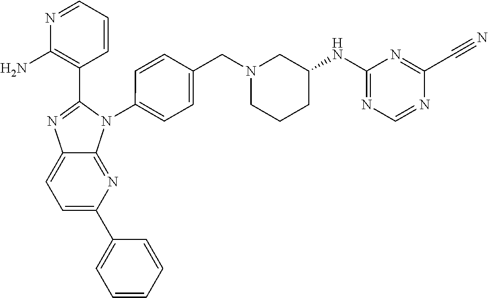 [SMILES: N#Cc1ncnc(N[C@@H]2CCCN(Cc3ccc(-n4c(-c5cccnc5N)nc5ccc(-c6ccccc6)nc54)cc3)C2)n1] | (R)-4-((1-(4-(2-(2-aminopyridin-3-yl)-5- phenyl-3H-imidazo[4,5-b]pyridin-3- yl)benzyl)piperidin-3-yl)amino)-1,3,5- triazine-2-carbonitrile |
| 338 | 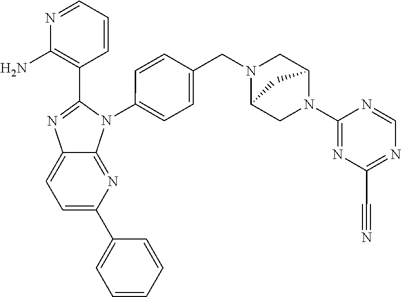 [SMILES: N#Cc1ncnc(N2C[C@@H]3C[C@H]2CN3Cc2ccc(-n3c(-c4cccnc4N)nc4ccc(-c5ccccc5)nc43)cc2)n1] | 4-((1S,4S)-5-(4-(2-(2-aminopyridin-3-yl)-5- phenyl-3H-imidazo[4,5-b]pyridin-3- yl)benzyl)-2,5-diazabicyclo[2.2.1]heptan-2- yl)-1,3,5-triazine-2-carbonitrile |
| 339 | 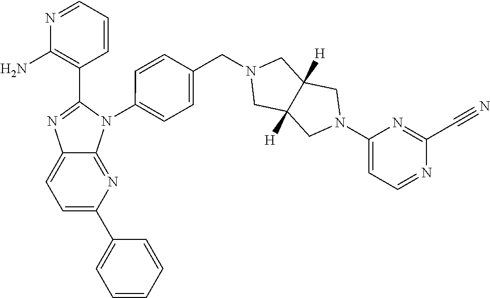 [SMILES: N#Cc1nccc(N2C[C@H]3CN(Cc4ccc(-n5c(-c6cccnc6N)nc6ccc(-c7ccccc7)nc65)cc4)C[C@H]3C2)n1] | 4-((3aR,6aS)-5-(4-(2-(2-aminopyridin-3- yl)-5-phenyl-3H-imidazo[4,5-b]pyridin-3- yl)benzyl)hexahydropyrrolo[3,4-c]pyrrol- 2(1H)-yl)pyrimidine-2-carbonitrile |
| 340 | 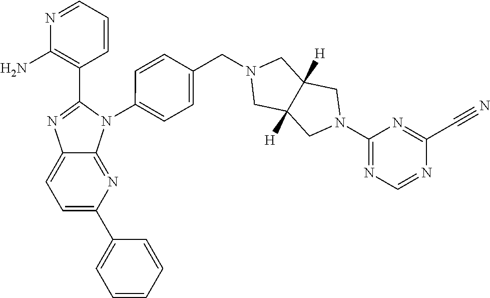 [SMILES: N#Cc1ncnc(N2C[C@H]3CN(Cc4ccc(-n5c(-c6cccnc6N)nc6ccc(-c7ccccc7)nc65)cc4)C[C@H]3C2)n1] | 4-((3aR,6aS)-5-(4-(2-(2-aminopyridin-3- yl)-5-phenyl-3H-imidazo[4,5-b]pyridin- 3- yl)benzyl)hexahydropyrrolo[3,4-c ]pyrrol- 2(1H)-yl)-1,3,5-triazine-2-carbonitrile |
| 341 | 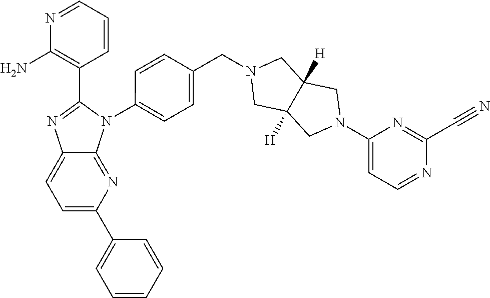 [SMILES: N#Cc1nccc(N2C[C@H]3CN(Cc4ccc(-n5c(-c6cccnc6N)nc6ccc(-c7ccccc7)nc65)cc4)C[C@@H]3C2)n1] | 4-((3aR,6aR)-5-(4-(2-(2-aminopyridin-3- yl)-5-phenyl-3H-imidazo[4,5-b]pyridin-3- yl)benzyl)hexahydropyrrolo[3,4-c]pyrrol- 2(1H)-yl)pyrimidine-2-carbonitrile |
| 342 | 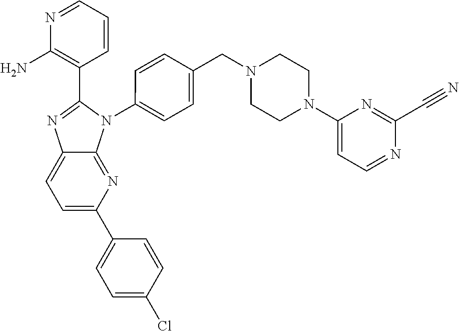 [SMILES: N#Cc1nccc(N2CCN(Cc3ccc(-n4c(-c5cccnc5N)nc5ccc(-c6ccc(Cl)cc6)nc54)cc3)CC2)n1] | 4-(4-(4-(2-(2-aminopyridin-3-yl)-5-(4- chlorophenyl)-3H-imidazo[4,5-b]pyridin-3- yl)benzyl)piperazin-1-yl)pyrimidine-2- carbonitrile |
| 343 | 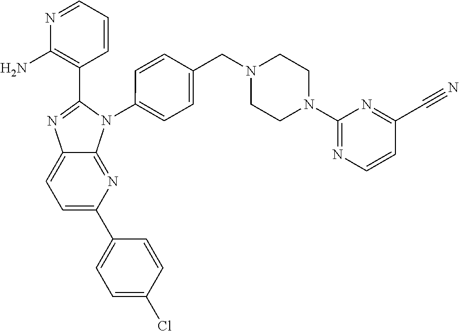 [SMILES: N#Cc1ccnc(N2CCN(Cc3ccc(-n4c(-c5cccnc5N)nc5ccc(-c6ccc(Cl)cc6)nc54)cc3)CC2)n1] | 2-(4-(4-(2-(2-aminopyridin-3-yl)-5-(4- chlorophenyl)-3H-imidazo[4,5-b]pyridin-3- yl)benzyl)piperazin-1-yl)pyrimidine-4- carbonitrile |
| 344 | 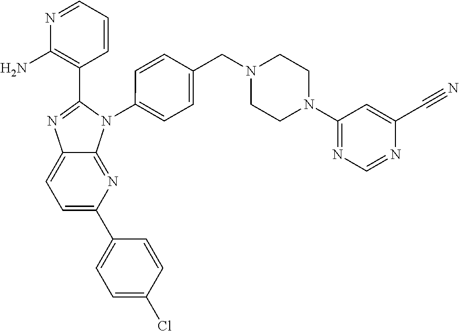 [SMILES: N#Cc1cc(N2CCN(Cc3ccc(-n4c(-c5cccnc5N)nc5ccc(-c6ccc(Cl)cc6)nc54)cc3)CC2)ncn1] | 6-(4-(4-(2-(2-aminopyridin-3-yl)-5-(4- chlorophenyl)-3H-imidazo[4,5-b]pyridin-3- yl)benzyl)piperazin-1-yl)pyrimidine-4- carbonitrile |
| 345 | 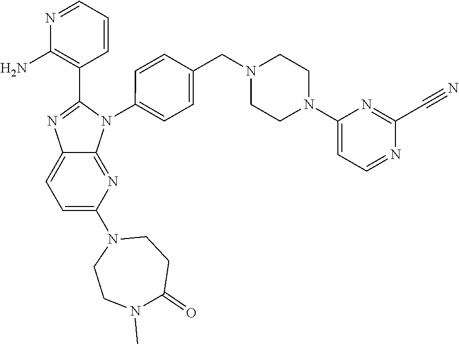 [SMILES: CN1CCN(c2ccc3nc(-c4cccnc4N)n(-c4ccc(CN5CCN(c6ccnc(C#N)n6)CC5)cc4)c3n2)CCC1=O] | 4-(4-(4-(2-(2-aminopyridin-3-yl)-5-(4- methyl-5-oxo-1,4-diazepan-1-yl)-3H- imidazo[4,5-b]pyridin-3- yl)benzyl)piperazin-1-yl)pyrimidine-2- carbonitrile |
| 346 | 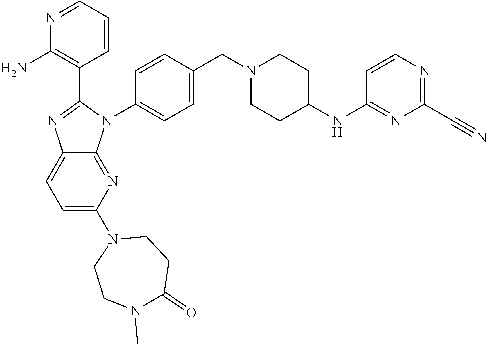 [SMILES: CN1CCN(c2ccc3nc(-c4cccnc4N)n(-c4ccc(CN5CCC(Nc6ccnc(C#N)n6)CC5)cc4)c3n2)CCC1=O] | 4-((1-(4-(2-(2-aminopyridin-3-yl)-5-(4- methyl-5-oxo-1,4-diazepan-1-yl)-3H- imidazo[4,5-b]pyridin-3- yl)benzyl)piperidin-4-yl)amino)pyrimidine- 2-carbonitrile |
| 347 | 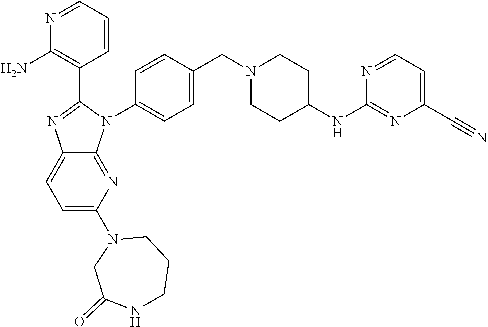 [SMILES: N#Cc1ccnc(NC2CCN(Cc3ccc(-n4c(-c5cccnc5N)nc5ccc(N6CCCNC(=O)C6)nc54)cc3)CC2)n1] | 2-((1-(4-(2-(2-aminopyridin-3-yl)-5-(3-oxo- 1,4-diazepan-1-yl)-3H-imidazo[4,5- b]pyridin-3-yl)benzyl)piperidin-4- yl)amino)pyrimidine-4-carbonitrile |
| 348 | 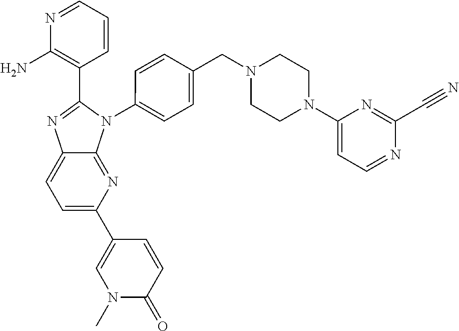 [SMILES: Cn1cc(-c2ccc3nc(-c4cccnc4N)n(-c4ccc(CN5CCN(c6ccnc(C#N)n6)CC5)cc4)c3n2)ccc1=O] | 4-(4-(4-(2-(2-aminopyridin-3-yl)-5-(1- methyl-6-oxo-1,6-dihydropyridin-3-yl)-3H- imidazo[4,5-b]pyridin-3- yl)benzyl)piperazin-1-yl)pyrimidine-2- carbonitrile |
| 349 | 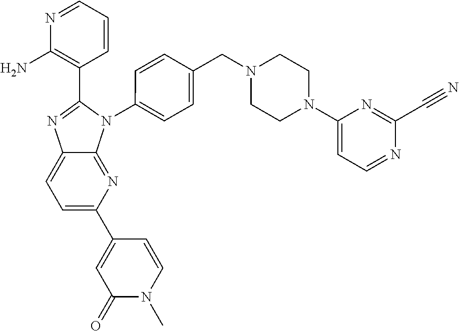 [SMILES: Cn1ccc(-c2ccc3nc(-c4cccnc4N)n(-c4ccc(CN5CCN(c6ccnc(C#N)n6)CC5)cc4)c3n2)cc1=O] | 4-(4-(4-(2-(2-aminopyridin-3-yl)-5-(1- methyl- 2-oxo-1,2-dihydropyridin-4-yl)-3H- imidazo[4,5-b]pyridin-3- yl)benzyl)piperazin-1-yl)pyrimidine-2- carbonitrile |
| 350 | 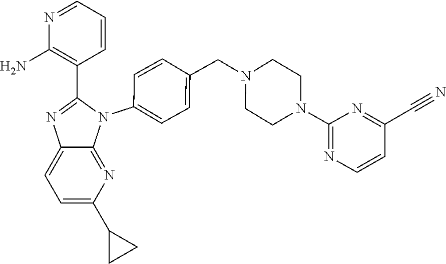 [SMILES: N#Cc1ccnc(N2CCN(Cc3ccc(-n4c(-c5cccnc5N)nc5ccc(C6CC6)nc54)cc3)CC2)n1] | 2-(4-(4-(2-(2-aminopyridin-3-yl)-5- cyclopropyl-3H-imidazo[4,5-b]pyridin-3- yl)benzyl)piperazin-1-yl)pyrimidine-4- carbonitrile |
| 351 | 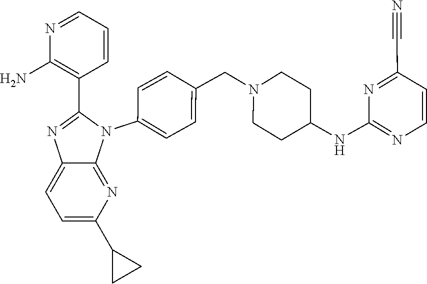 [SMILES: N#Cc1ccnc(NC2CCN(Cc3ccc(-n4c(-c5cccnc5N)nc5ccc(C6CC6)nc54)cc3)CC2)n1] | 2-((1-(4-(2-(2-aminopyridin-3-yl)-5- cyclopropyl-3H-imidazo[4,5-b]pyridin-3- yl)benzyl)piperidin-4-yl)amino)pyrimidine- 4-carbonitrile |
| 352 | 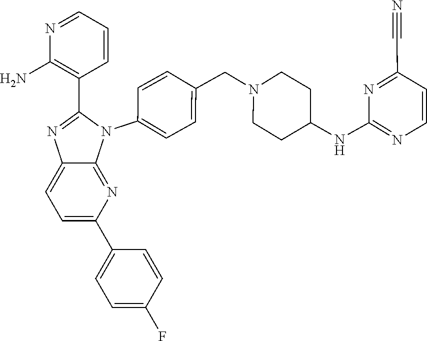 [SMILES: N#Cc1ccnc(NC2CCN(Cc3ccc(-n4c(-c5cccnc5N)nc5ccc(-c6ccc(F)cc6)nc54)cc3)CC2)n1] | 2-((1-(4-(2-(2-aminopyridin-3-yl)-5-(4- fluorophenyl)-3H-imidazo[4,5-b]pyridin-3- yl)benzyl)piperidin-4-yl)amino)pyrimidine- 4-carbonitrile |
| 353 | 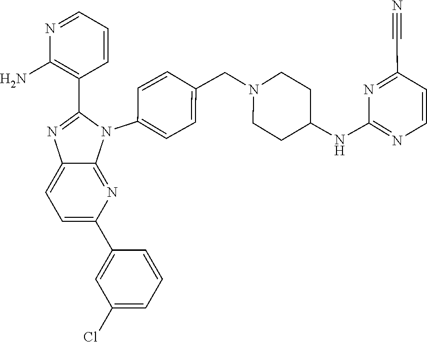 [SMILES: N#Cc1ccnc(NC2CCN(Cc3ccc(-n4c(-c5cccnc5N)nc5ccc(-c6cccc(Cl)c6)nc54)cc3)CC2)n1] | 4-((1-(4-(2-(2-aminopyridin-3-yl)-5-(3- chlorophenyl)-3H-imidazo[4,5-b]pyridin-3- yl)benzyl)piperidin-4-yl)amino)pyrimidine- 2-carbonitrile |
| 354 | 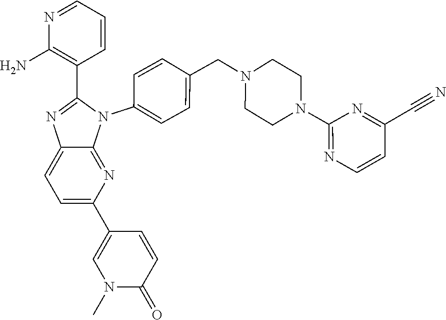 [SMILES: Cn1cc(-c2ccc3nc(-c4cccnc4N)n(-c4ccc(CN5CCN(c6nccc(C#N)n6)CC5)cc4)c3n2)ccc1=O] | 2-(4-(4-(2-(2-aminopyridin-3-yl)-5-(1- methyl-6-oxo-1,6-dihydropyridin-3-yl)-3H- imidazo[4,5-b]pyridin-3- yl)benzyl)piperazin-1-yl)pyrimidine-4- carbonitrile |
| 355 | 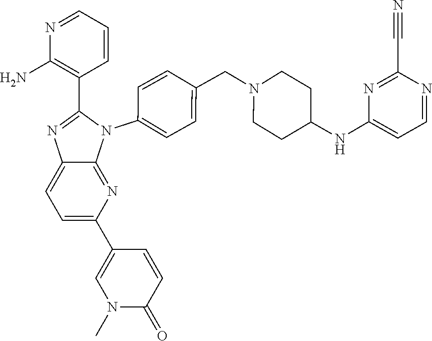 [SMILES: Cn1cc(-c2ccc3nc(-c4cccnc4N)n(-c4ccc(CN5CCC(Nc6ccnc(C#N)n6)CC5)cc4)c3n2)ccc1=O] | 4-((1-(4-(2-(2-aminopyridin-3-yl)-5-(1- methyl-6-oxo-1,6-dihydropyridin-3-yl)-3H- imidazo[4,5-b]pyridin-3- yl)benzyl)piperidin-4-yl)amino)pyrimidine- 2-carbonitrile |
| 356 | 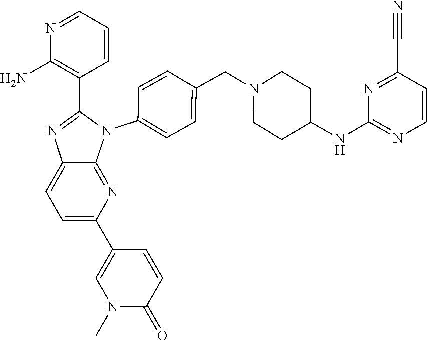 [SMILES: Cn1cc(-c2ccc3nc(-c4cccnc4N)n(-c4ccc(CN5CCC(Nc6nccc(C#N)n6)CC5)cc4)c3n2)ccc1=O] | 2-((1-(4-(2-(2-aminopyridin-3-yl)-5-(1- methyl-6-oxo-1,6-dihydropyridin-3-yl)-3H- imidazo[4,5-b]pyridin-3- yl)benzyl)piperidin-4-yl)amino)pyrimidine- 4-carbonitrile |
| 357 | 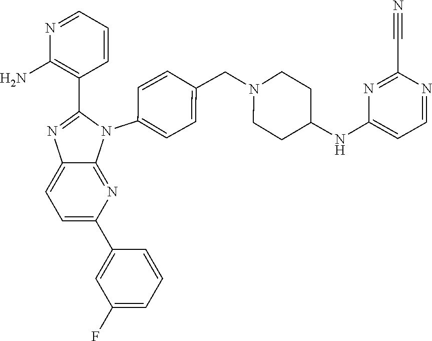 [SMILES: N#Cc1nccc(NC2CCN(Cc3ccc(-n4c(-c5cccnc5N)nc5ccc(-c6cccc(F)c6)nc54)cc3)CC2)n1] | 4-((1-(4-(2-(2-aminopyridin-3-yl)-5-(3- fluorophenyl)-3H-imidazo[4,5-b]pyridin-3- yl)benzyl)piperidin-4-yl)amino)pyrimidine- 2-carbonitrile |
| 358 | 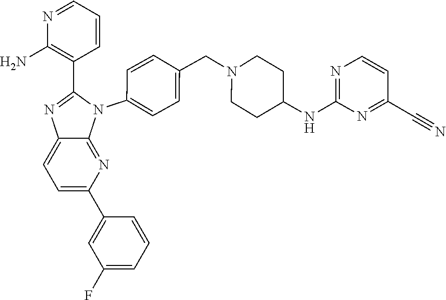 [SMILES: N#Cc1ccnc(NC2CCN(Cc3ccc(-n4c(-c5cccnc5N)nc5ccc(-c6cccc(F)c6)nc54)cc3)CC2)n1] | 2-((1-(4-(2-(2-aminopyridin-3-yl)-5-(3- fluorophenyl)-3H-imidazo[4,5-b]pyridin-3- yl)benzyl)piperidin-4-yl)amino)pyrimidine- 4-carbonitrile |
| 359 | 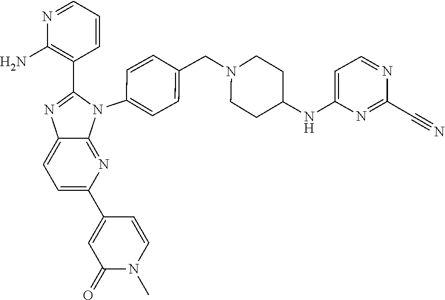 [SMILES: Cn1ccc(-c2ccc3nc(-c4cccnc4N)n(-c4ccc(CN5CCC(Nc6ccnc(C#N)n6)CC5)cc4)c3n2)cc1=O] | 4-((1-(4-(2-(2-aminopyridin-3-yl)-5-(1- methyl-2-oxo-1,2-dihydropyridin-4-yl)-3H- imidazo[4,5-b]pyridin-3- yl)benzyl)piperidin-4-yl)amino)pyrimidine- 2-carbonitrile |
| 360 | 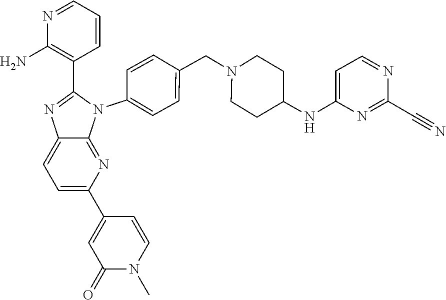 [SMILES: Cn1ccc(-c2ccc3nc(-c4cccnc4N)n(-c4ccc(CN5CCC(Nc6ccnc(C#N)n6)CC5)cc4)c3n2)cc1=O] | 2-((1-(4-(2-(2-aminopyridin-3-yl)-5-(1- methyl-2-oxo-1,2-dihydropyridin-4-yl)-3H- imidazo[4,5-b]pyridin-3- yl)benzyl)piperidin-4-yl)amino)pyrimidine- 4-carbonitrile |
| 361 | 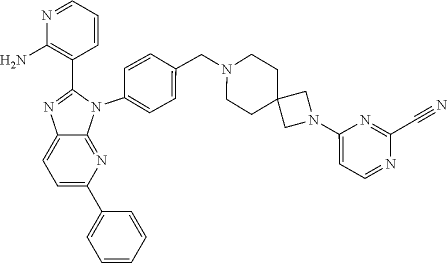 [SMILES: N#Cc1nccc(N2CC3(CCN(Cc4ccc(-n5c(-c6cccnc6N)nc6ccc(-c7ccccc7)nc65)cc4)CC3)C2)n1] | 4-(7-(4-(2-(2-aminopyridin-3-yl)-5-phenyl- 3H-imidazo[4,5-b]pyridin-3-yl)benzyl)-2,7- diazaspiro[3.5]nonan-2-yl)pyrimidine-2- carbonitrile |
| 362 | 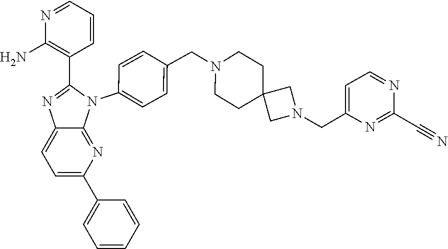 [SMILES: N#Cc1nccc(CN2CC3(CCN(Cc4ccc(-n5c(-c6cccnc6N)nc6ccc(-c7ccccc7)nc65)cc4)CC3)C2)n1] | 4-((7-(4-(2-(2-aminopyridin-3-yl)-5-phenyl- 3H-imidazo[4,5-b]pyridin-3-yl)benzyl)-7- azaspiro[3.5]nonan-2-yl)amino)pyrimidine- 2-carbonitrile |
| 363 | 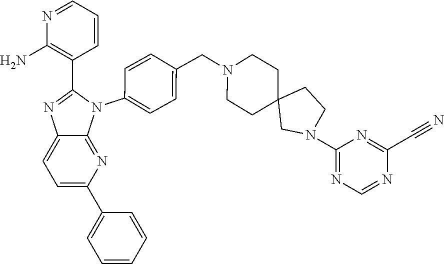 [SMILES: N#Cc1ncnc(N2CCC3(CCN(Cc4ccc(-n5c(-c6cccnc6N)nc6ccc(-c7ccccc7)nc65)cc4)CC3)C2)n1] | 4-(8-(4-(2-(2-aminopyridin-3-yl)-5-phenyl- 3H-imidazo[4,5-b]pyridin-3-yl)benzyl)-2,8- diazaspiro[4.5]decan-2-yl)-1,3,5-triazine-2- carbonitrile |
| 364 | 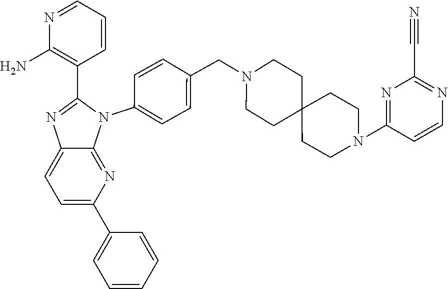 [SMILES: N#Cc1nccc(N2CCC3(CCN(Cc4ccc(-n5c(-c6cccnc6N)nc6ccc(-c7ccccc7)nc65)cc4)CC3)CC2)n1] | 4-(9-(4-(2-(2-aminopyridin-3-yl)-5-phenyl- 3H-imidazo[4,5-b]pyridin-3-yl)benzyl)-3,9- diazaspiro[5.5 ]undecan-3-yl)pyrimidine-2- carbonitrile |
| 365 | 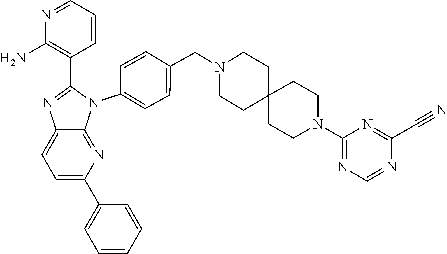 [SMILES: N#Cc1ncnc(N2CCC3(CCN(Cc4ccc(-n5c(-c6cccnc6N)nc6ccc(-c7ccccc7)nc65)cc4)CC3)CC2)n1] | 4-(9-(4-(2-(2-aminopyridin-3-yl)-5-phenyl- 3H-imidazo[4,5-b]pyridin-3-yl)benzyl)-3,9- diazaspiro[5.5]undecan-3-yl)-1,3,5-triazine- 2-carbonitrile |
| 366 | 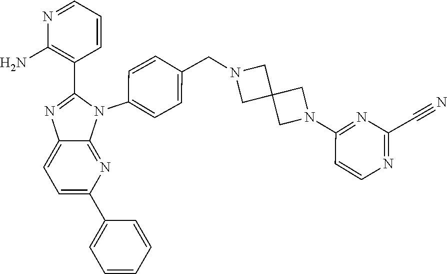 [SMILES: N#Cc1nccc(N2CC3(CN(Cc4ccc(-n5c(-c6cccnc6N)nc6ccc(-c7ccccc7)nc65)cc4)C3)C2)n1] | 4-(6-(4-(2-(2-aminopyridin-3-yl)-5-phenyl- 3H-imidazo[4,5-b]pyridin-3-yl)benzyl)-2,6- diazaspiro[3.3]heptan-2-yl)pyrimidine-2- carbonitrile |
| 367 | 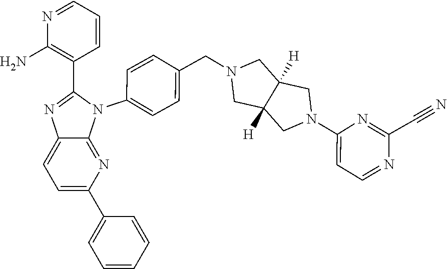 [SMILES: N#Cc1nccc(N2C[C@@H]3CN(Cc4ccc(-n5c(-c6cccnc6N)nc6ccc(-c7ccccc7)nc65)cc4)C[C@H]3C2)n1] | 4-((3aS,6aS)-5-(4-(2-(2-aminopyridin-3-yl)- 5-phenyl-3H-imidazo[4,5-b]pyridin-3- yl)benzyl)hexahydropyrrolo[3,4-c]pyrrol- 2(1H)-yl)pyrimidine-2-carbonitrile |
| 368 | 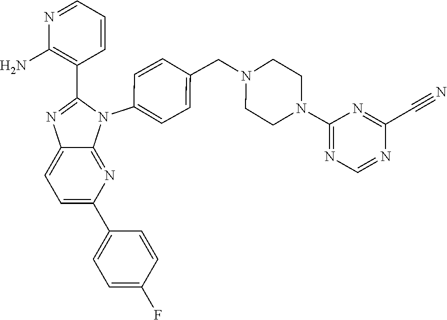 [SMILES: N#Cc1ncnc(N2CCN(Cc3ccc(-n4c(-c5cccnc5N)nc5ccc(-c6ccc(F)cc6)nc54)cc3)CC2)n1] | 4-(4-(4-(2-(2-aminopyridin-3-yl)-5-(4- fluorophenyl)-3H-imidazo[4,5-b]pyridin-3- yl)benzyl)piperazin-1-yl)-1,3,5-triazine-2- carbonitrile |
| 369 | 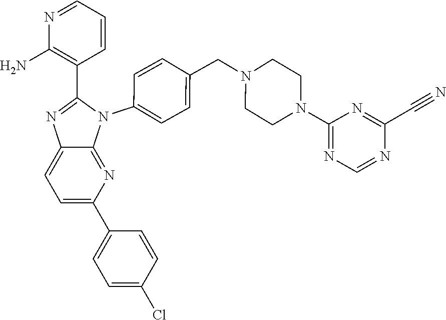 [SMILES: N#Cc1ncnc(N2CCN(Cc3ccc(-n4c(-c5cccnc5N)nc5ccc(-c6ccc(Cl)cc6)nc54)cc3)CC2)n1] | 4-(4-(4-(2-(2-aminopyridin-3-yl)-5-(4- chlorophenyl)-3H-imidazo[4,5-b]pyridin-3- yl)benzyl)piperazin-1-yl)-1,3,5-triazine-2- carbonitrile |
| 370 | 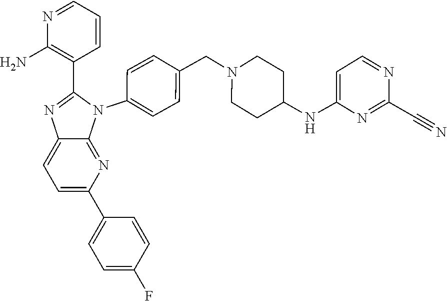 [SMILES: N#Cc1nccc(NC2CCN(Cc3ccc(-n4c(-c5cccnc5N)nc5ccc(-c6ccc(F)cc6)nc54)cc3)CC2)n1] | 4-((1-(4-(2-(2-aminopyridin-3-yl)-5-(4- fluorophenyl)-3H-imidazo[4,5-b]pyridin-3- yl)benzyl)piperidin-4-yl)amino)pyrimidine- 2-carbonitrile |
| 371 | 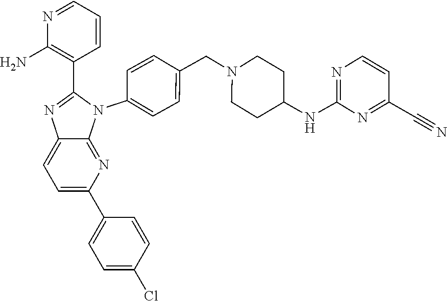 [SMILES: N#Cc1ccnc(NC2CCN(Cc3ccc(-n4c(-c5cccnc5N)nc5ccc(-c6ccc(Cl)cc6)nc54)cc3)CC2)n1] | 2-((1-(4-(2-(2-aminopyridin-3-yl)-5-(4- chlorophenyl)-3H-imidazo[4,5-b]pyridin-3- yl)benzyl)piperidin-4-yl)amino)pyrimidine- 4-carbonitrile |
| 372 | 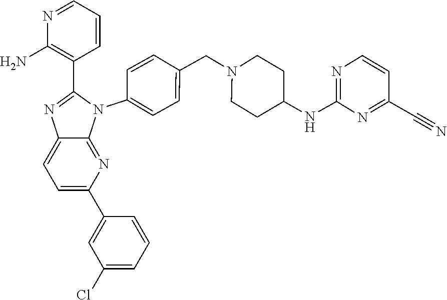 [SMILES: N#Cc1ccnc(NC2CCN(Cc3ccc(-n4c(-c5cccnc5N)nc5ccc(-c6cccc(Cl)c6)nc54)cc3)CC2)n1] | 2-((1-(4-(2-(2-aminopyridin-3-yl)-5-(3- chlorophenyl)-3H-imidazo[4,5-b]pyridin-3- yl)benzyl)piperidin-4-yl)amino)pyrimidine- 4-carbonitrile |
| 373 | 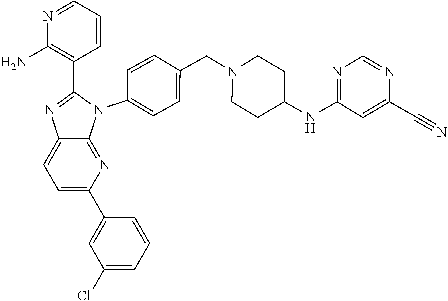 [SMILES: N#Cc1cc(NC2CCN(Cc3ccc(-n4c(-c5cccnc5N)nc5ccc(-c6cccc(Cl)c6)nc54)cc3)CC2)ncn1] | 6-((1-(4-(2-(2-aminopyridin-3-yl)-5-(3- chlorophenyl)-3H-imidazo[4,5-b]pyridin-3- yl)benzyl)piperidin-4-yl)amino)pyrimidine- 4-carbonitrile |
| 374 | 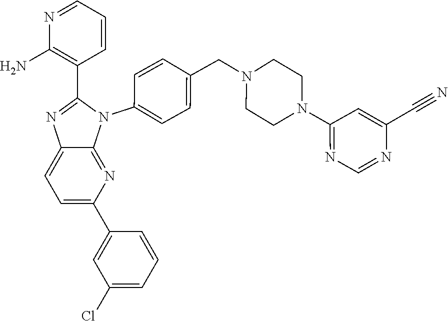 [SMILES: N#Cc1cc(N2CCN(Cc3ccc(-n4c(-c5cccnc5N)nc5ccc(-c6cccc(Cl)c6)nc54)cc3)CC2)ncn1] | 6-(4-(4-(2-(2-aminopyridin-3-yl)-5-(3- chlorophenyl)-3H-imidazo[4,5-b]pyridin-3- yl)benzyl)piperazin-1-yl)pyrimidine-4- carbonitrile |
| 375 | 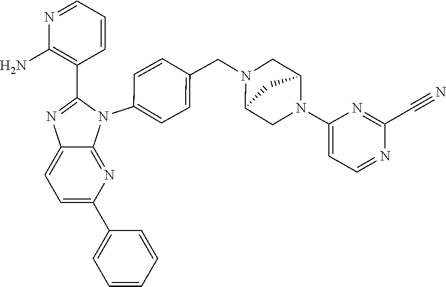 [SMILES: N#Cc1nccc(N2C[C@@H]3C[C@H]2CN3Cc2ccc(-n3c(-c4cccnc4N)nc4ccc(-c5ccccc5)nc43)cc2)n1] | 4-((1S,4S)-5-(4-(2-(2-aminopyridin-3-yl)-5- phenyl-3H-imidazo[4,5-b]pyridin-3- yl)benzyl)-2,5-diazabicyclo[2.2.1]heptan-2- yl)pyrimidine-2-carbonitrile |
| 376 | 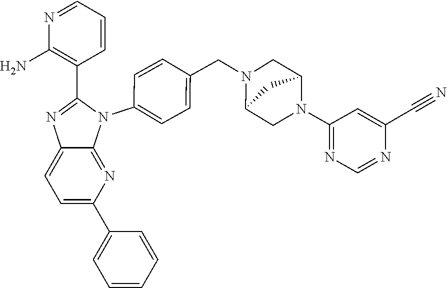 [SMILES: N#Cc1cc(N2C[C@@H]3C[C@H]2CN3Cc2ccc(-n3c(-c4cccnc4N)nc4ccc(-c5ccccc5)nc43)cc2)ncn1] | 6-((1S,4S)-5-(4-(2-(2-aminopyridin-3-yl)-5- phenyl-3H-imidazo[4,5-b]pyridin-3- yl)benzyl)-2,5-diazabicyclo[2.2.1]heptan-2- yl)pyrimidine-4-carbonitrile |
| 377 | 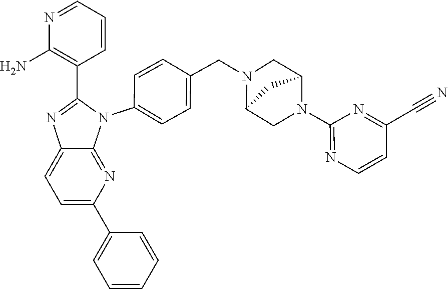 [SMILES: N#Cc1ccnc(N2C[C@@H]3C[C@H]2CN3Cc2ccc(-n3c(-c4cccnc4N)nc4ccc(-c5ccccc5)nc43)cc2)n1] | 2-((1S,4S)-5-(4-(2-(2-aminopyridin-3-yl)-5- phenyl-3H-imidazo[4,5-b]pyridin-3- yl)benzyl)-2,5-diazabicyclo[2.2.1]heptan-2- yl)pyrimidine-4-carbonitrile |
| 378 | 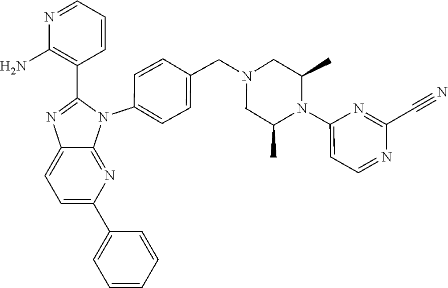 [SMILES: C[C@@H]1CN(Cc2ccc(-n3c(-c4cccnc4N)nc4ccc(-c5ccccc5)nc43)cc2)C[C@H](C)N1c1ccnc(C#N)n1] | 4-((2S,6R)-4-(4-(2-(2-aminopyridin-3-yl)-5- phenyl-3H-imidazo[4,5-b]pyridin-3- yl)benzyl)-2,6-dimethylpiperazin-1- yl)pyrimidine-2-carbonitrile |
| 379 | 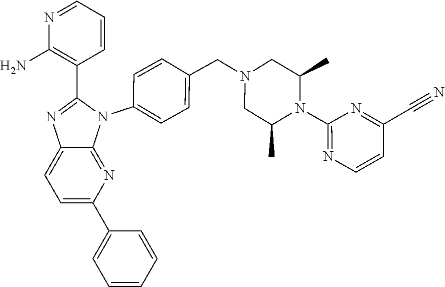 [SMILES: C[C@@H]1CN(Cc2ccc(-n3c(-c4cccnc4N)nc4ccc(-c5ccccc5)nc43)cc2)C[C@H](C)N1c1nccc(C#N)n1] | 2-((2S,6R)-4-(4-(2-(2-aminopyridin-3-yl)-5- phenyl-3H-imidazo[4,5-b]pyridin-3- yl)benzyl)-2,6-dimethylpiperazin-1- yl)pyrimidine-4-carbonitrile |
| 380 | 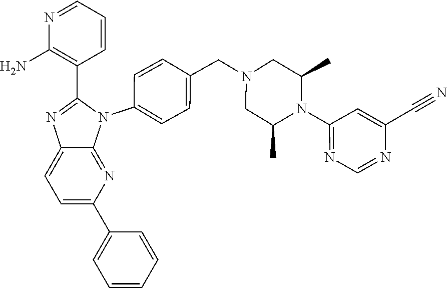 [SMILES: C[C@@H]1CN(Cc2ccc(-n3c(-c4cccnc4N)nc4ccc(-c5ccccc5)nc43)cc2)C[C@H](C)N1c1cc(C#N)ncn1] | 6-((2S,6R)-4-(4-(2-(2-aminopyridin-3-yl)-5- phenyl-3H-imidazo[4,5-b]pyridin-3- yl)benzyl)-2,6-dimethylpiperazin-1- yl)pyrimidine-4-carbonitrile |
| 381 | 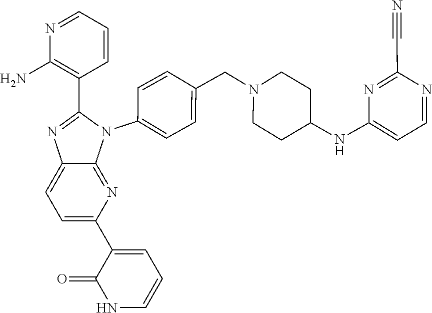 [SMILES: N#Cc1nccc(NC2CCN(Cc3ccc(-n4c(-c5cccnc5N)nc5ccc(-c6ccc[nH]c6=O)nc54)cc3)CC2)n1] | 4-((1-(4-(2-(2-aminopyridin-3-yl)-5-(2-oxo- 1,2-dihydropyridin-3-yl)-3H-imidazo[4,5- b]pyridin-3-yl)benzyl)piperidin-4- yl)amino)pyrimidine-2-carbonitrile |
| 382 | 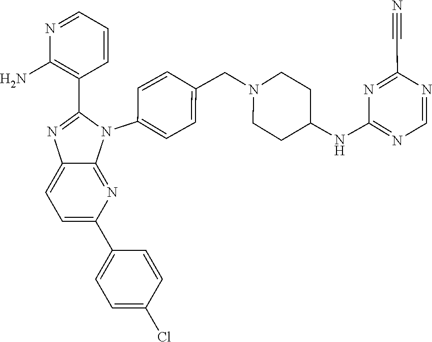 [SMILES: N#Cc1ncnc(NC2CCN(Cc3ccc(-n4c(-c5cccnc5N)nc5ccc(-c6ccc(Cl)cc6)nc54)cc3)CC2)n1] | 4-((1-(4-(2-(2-aminopyridin-3-yl)-5-(4- chlorophenyl)-3H-imidazo[4,5-b]pyridin-3- yl)benzyl)piperidin-4-yl)amino)-1,3,5- triazine-2-carbonitrile |
| 383 | 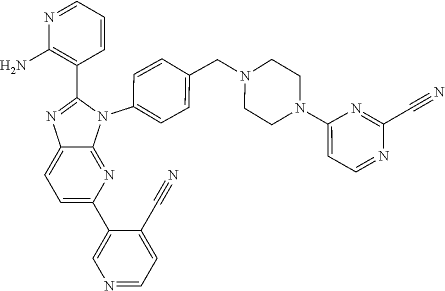 [SMILES: N#Cc1nccc(N2CCN(Cc3ccc(-n4c(-c5cccnc5N)nc5ccc(-c6cnccc6C#N)nc54)cc3)CC2)n1] | 4-(4-(4-(2-(2-aminopyridin-3-yl)-5-(4- cyanopyridin-3-yl)-3H-imidazo[4,5- b]pyridin-3-yl)benzyl)piperazin-1- yl)pyrimidine-2-carbonitrile |
| 384 | 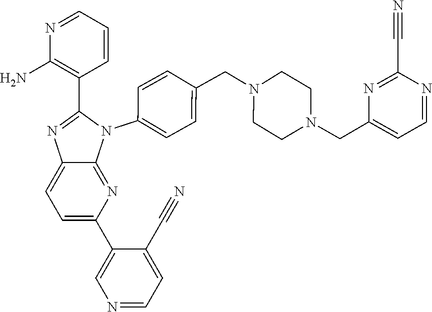 [SMILES: N#Cc1nccc(CN2CCN(Cc3ccc(-n4c(-c5cccnc5N)nc5ccc(-c6cnccc6C#N)nc54)cc3)CC2)n1] | 4-((1-(4-(2-(2-aminopyridin-3-yl)-5-(4- cyanopyridin-3-yl)-3H-imidazo[4,5- b]pyridin-3-yl)benzyl)piperidin-4- yl)amino)pyrimidine-2-carbonitrile |
| 385 | 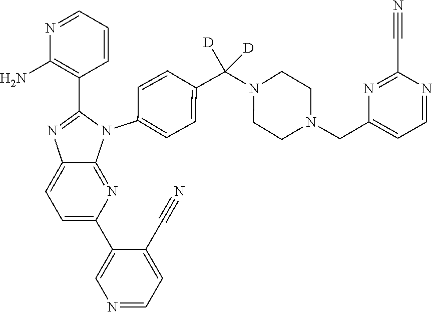 [SMILES: [2H]C([2H])(c1ccc(-n2c(-c3cccnc3N)nc3ccc(-c4cnccc4C#N)nc32)cc1)N1CCN(Cc2ccnc(C#N)n2)CC1] | 4-((1-((4-(2-(2-aminopyridin-3-yl)-3H- imidazo[4,5-b]pyridin-3-yl)phenyl)methyl- d2)piperidin-4-yl)amino)pyrimidine-2- carbonitrile |
| 386 | 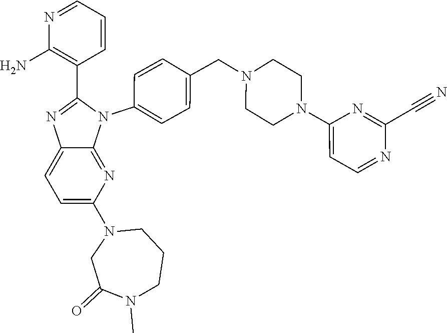 [SMILES: CN1CCCN(c2ccc3nc(-c4cccnc4N)n(-c4ccc(CN5CCN(c6ccnc(C#N)n6)CC5)cc4)c3n2)CC1=O] | 4-(4-(4-(2-(2-aminopyridin-3-yl)-5-(4- methyl-3-oxo-1,4-diazepan-1-yl)-3H- imidazo[4,5-b]pyridin-3- yl)benzyl)piperazin-1-yl)pyrimidine-2- carbonitrile |
| 387 | 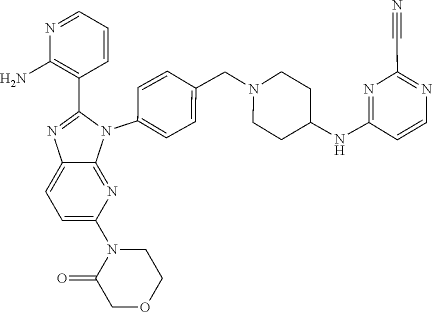 [SMILES: N#Cc1nccc(NC2CCN(Cc3ccc(-n4c(-c5cccnc5N)nc5ccc(N6CCOCC6=O)nc54)cc3)CC2)n1] | 4-((1-(4-(2-(2-aminopyridin-3-yl)-5-(3- oxomorpholino)-3H-imidazo[4,5-b]pyridin- 3-yl)benzyl)piperidin-4- yl)amino)pyrimidine-2-carbonitrile |
| 388 | 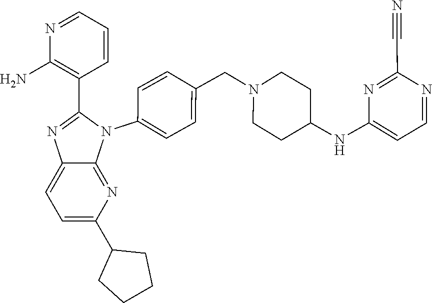 [SMILES: N#Cc1nccc(NC2CCN(Cc3ccc(-n4c(-c5cccnc5N)nc5ccc(C6CCCC6)nc54)cc3)CC2)n1] | 4-((1-(4-(2-(2-aminopyridin-3-yl)-5- cyclopentyl-3H-imidazo[4,5-b]pyridin-3- yl)benzyl)piperidin-4-yl)amino)pyrimidine- 2-carbonitrile |
| 389 | 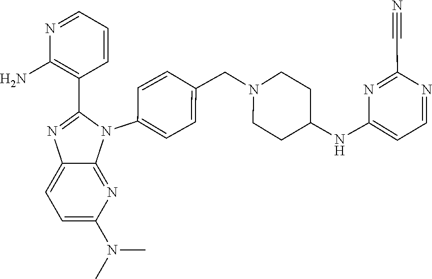 [SMILES: CN(C)c1ccc2nc(-c3cccnc3N)n(-c3ccc(CN4CCC(Nc5ccnc(C#N)n5)CC4)cc3)c2n1] | 4-((1-(4-(2-(2-aminopyridin-3-yl)-5- (dimethylamino)-3H-imidazo[4,5-b]pyridin- 3-yl)benzyl)piperidin-4- yl)amino)pyrimidine-2-carbonitrile |
| 390 | 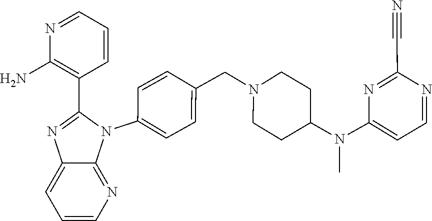 [SMILES: CN(c1ccnc(C#N)n1)C1CCN(Cc2ccc(-n3c(-c4cccnc4N)nc4cccnc43)cc2)CC1] | 4-((1-(4-(2-(2-aminopyridin-3-yl)-3H- imidazo[4,5-b]pyridin-3- yl)benzyl)piperidin-4- yl)(methyl)amino)pyrimidine-2-carbonitrile |
| 391 | 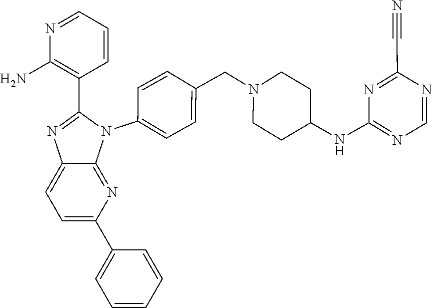 [SMILES: N#Cc1ncnc(NC2CCN(Cc3ccc(-n4c(-c5cccnc5N)nc5ccc(-c6ccccc6)nc54)cc3)CC2)n1] | 4-((1-(4-(2-(2-aminopyridin-3-yl)-5-phenyl- 3H-imidazo[4,5-b]pyridin-3- yl)benzyl)piperidin-4-yl)amino)-1,3,5- triazine-2-carbonitrile |
| 392 | 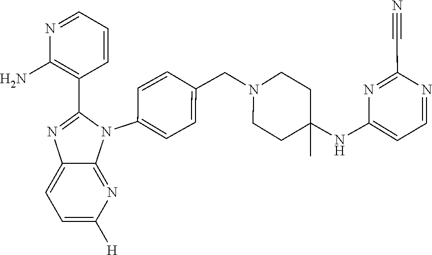 [SMILES: CC1(Nc2ccnc(C#N)n2)CCN(Cc2ccc(-n3c(-c4cccnc4N)nc4cccnc43)cc2)CC1] | 4-((1-(4-(2-(2-aminopyridin-3-yl)-3H- imidazo[4,5-b]pyridin-3-yl)benzyl)-4- methylpiperidin-4-yl)amino)pyrimidine-2- carbonitrile |
| 393 | 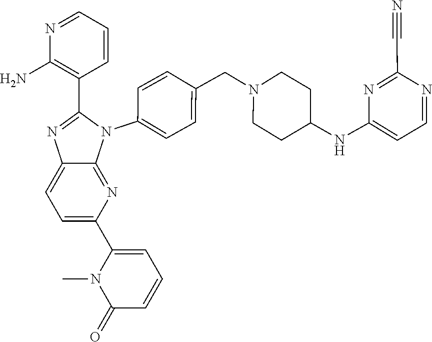 [SMILES: Cn1c(-c2ccc3nc(-c4cccnc4N)n(-c4ccc(CN5CCC(Nc6ccnc(C#N)n6)CC5)cc4)c3n2)cccc1=O] | 4-((1-(4-(2-(2-aminopyridin-3-yl)-5-(1- methyl-6-oxo-1,6-dihydropyridin-2-yl)-3H- imidazo[4,5-b]pyridin-3- yl)benzyl)piperidin-4-yl)amino)pyrimidine- 2-carbonitrile |
| 394 | 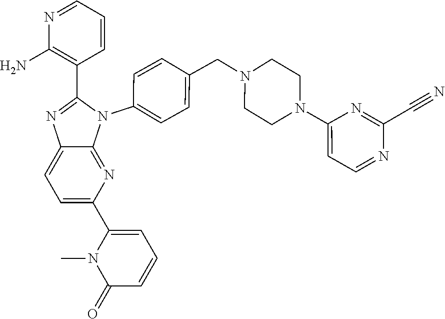 [SMILES: Cn1c(-c2ccc3nc(-c4cccnc4N)n(-c4ccc(CN5CCN(c6ccnc(C#N)n6)CC5)cc4)c3n2)cccc1=O] | 4-(4-(4-(2-(2-aminopyridin-3-yl)-5-(1- methyl-6-oxo-1,6-dihydropyridin-2-yl)-3H- imidazo[4,5-b]pyridin-3- yl)benzyl)piperazin-1-yl)pyrimidine-2- carbonitrile |
| 395 | 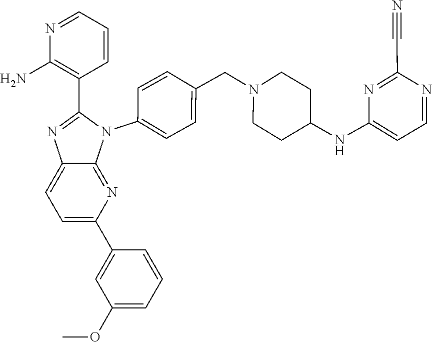 [SMILES: COc1cccc(-c2ccc3nc(-c4cccnc4N)n(-c4ccc(CN5CCC(Nc6ccnc(C#N)n6)CC5)cc4)c3n2)c1] | 4-((1-(4-(2-(2-aminopyridin-3-yl)-5-(3- methoxyphenyl)-3H-imidazo[4,5-b]pyridin- 3-yl)benzyl)piperidin-4- yl)amino)pyrimidine-2-carbonitrile |
| 396 | 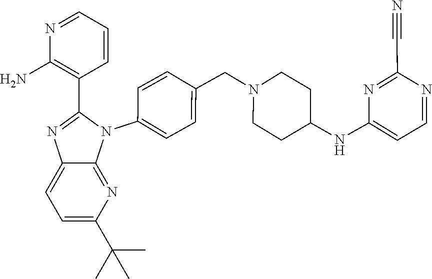 [SMILES: CC(C)(C)c1ccc2nc(-c3cccnc3N)n(-c3ccc(CN4CCC(Nc5ccnc(C#N)n5)CC4)cc3)c2n1] | 4-((1-(4-(2-(2-aminopyridin-3-yl)-5-(tert- butyl)-3H-imidazo[4,5-b]pyridin-3- yl)benzyl)piperidin-4-yl)amino)pyrimidine- 2-carbonitrile |
| 397 | 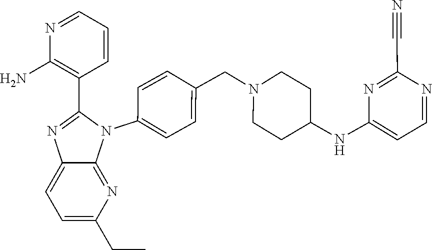 [SMILES: CCc1ccc2nc(-c3cccnc3N)n(-c3ccc(CN4CCC(Nc5ccnc(C#N)n5)CC4)cc3)c2n1] | 4-((1-(4-(2-(2-aminopyridin-3-yl)-5-ethyl- 3H-imidazo[4,5-b]pyridin-3- yl)benzyl)piperidin-4-yl)amino)pyrimidine- 2-carbonitrile |
| 398 | 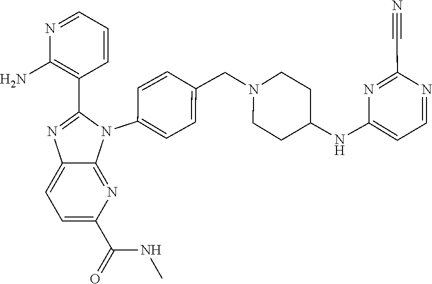 [SMILES: CNC(=O)c1ccc2nc(-c3cccnc3N)n(-c3ccc(CN4CCC(Nc5ccnc(C#N)n5)CC4)cc3)c2n1] | 2-(2-aminopyridin-3-yl)-3-(4-((4-((2- cyanopyrimidin-4-yl)amino)piperidin-1- yl)methyl)phenyl)-N-methyl-3H- imidazo[4,5-b]pyridine-5-carboxamide |
| 399 | 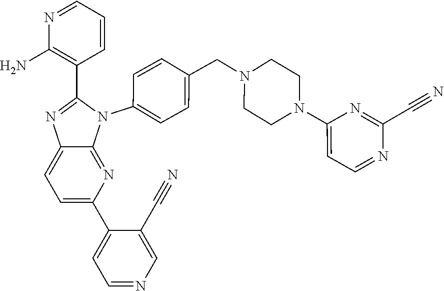 [SMILES: N#Cc1nccc(N2CCN(Cc3ccc(-n4c(-c5cccnc5N)nc5ccc(-c6ccncc6C#N)nc54)cc3)CC2)n1] | 4-(4- (4-(2-(2-aminopyridin-3-yl)-5-(3- cyanopyridin-4-yl)-3H-imidazo[4,5- b]pyridin-3-yl)benzyl)piperazin-1- yl)pyrimidine-2-carbonitrile |
| 400 | 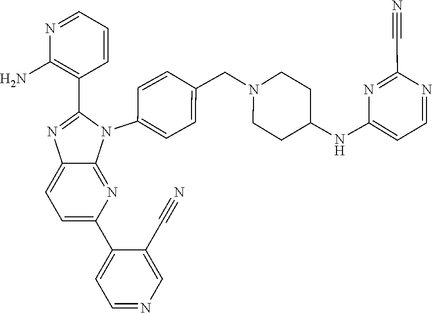 [SMILES: N#Cc1nccc(NC2CCN(Cc3ccc(-n4c(-c5cccnc5N)nc5ccc(-c6ccncc6C#N)nc54)cc3)CC2)n1] | 4-((1-(4-(2-(2-aminopyridin-3-yl)-5-(3- cyanopyridin-4-yl)-3H-imidazo[4,5- b]pyridin-3-yl)benzyl)piperidin-4- yl)amino)pyrimidine-2-carbonitrile |
| 401 | 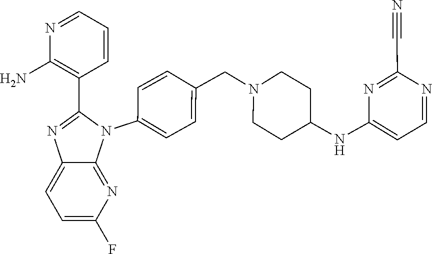 [SMILES: N#Cc1nccc(NC2CCN(Cc3ccc(-n4c(-c5cccnc5N)nc5ccc(F)nc54)cc3)CC2)n1] | 4-((1-(4-(2-(2-aminopyridin-3-yl)-5-fluoro- 3H-imidazo[4,5-b]pyridin-3- yl)benzyl)piperidin-4-yl)amino)pyrimidine- 2-carbonitrile |
| 402 | 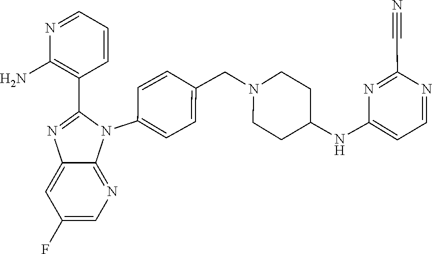 [SMILES: N#Cc1nccc(NC2CCN(Cc3ccc(-n4c(-c5cccnc5N)nc5cc(F)cnc54)cc3)CC2)n1] | 4-((1-(4-(2-(2-aminopyridin-3-yl)-6-fluoro- 3H-imidazo[4,5-b]pyridin-3- yl)benzyl)piperidin-4-yl)amino)pyrimidine- 2-carbonitrile |
| 403 | 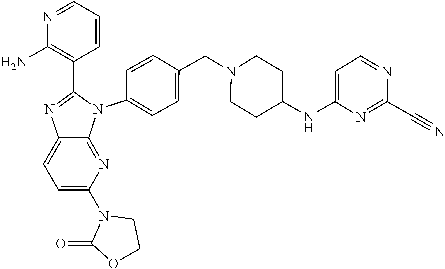 [SMILES: N#Cc1nccc(NC2CCN(Cc3ccc(-n4c(-c5cccnc5N)nc5ccc(N6CCOC6=O)nc54)cc3)CC2)n1] | 4-((1-(4-(2-(2-aminopyridin-3-yl)-5-(2- oxooxazolidin-3-yl)-3H-imidazo[4,5- b]pyridin-3-yl)benzyl)piperidin-4- yl)amino)pyrimidine-2-carbonitrile |
| 404 | 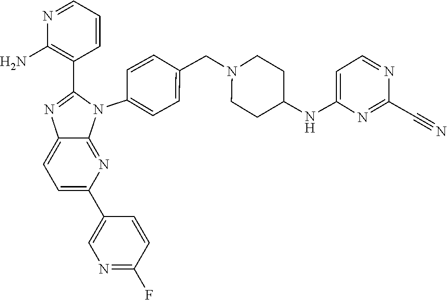 [SMILES: N#Cc1nccc(NC2CCN(Cc3ccc(-n4c(-c5cccnc5N)nc5ccc(-c6ccc(F)nc6)nc54)cc3)CC2)n1] | 4-((1-(4-(2-(2-aminopyridin-3-yl)-5-(6- fluoropyridin-3-yl)-3H-imidazo[4,5- b]pyridin-3-yl)benzyl)piperidin-4- yl)amino)pyrimidine-2-carbonitrile |
| 405 | 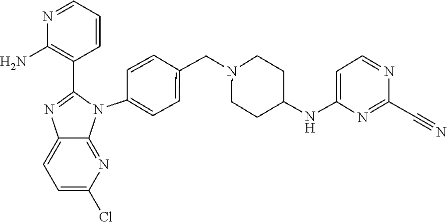 [SMILES: N#Cc1nccc(NC2CCN(Cc3ccc(-n4c(-c5cccnc5N)nc5ccc(Cl)nc54)cc3)CC2)n1] | 4-((1-(4-(2-(2-aminopyridin-3-yl)-5-chloro- 3H-imidazo[4,5-b ]pyridin-3- yl)benzyl)piperidin-4-yl)amino)pyrimidine- 2-carbonitrile |
| 406 | 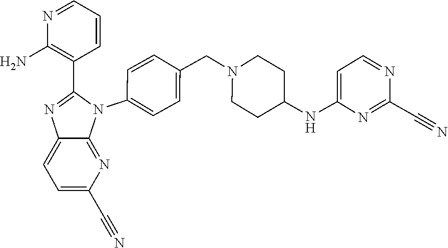 [SMILES: N#Cc1ccc2nc(-c3cccnc3N)n(-c3ccc(CN4CCC(Nc5ccnc(C#N)n5)CC4)cc3)c2n1] | 2-(2-aminopyridin-3-yl)-3-(4-((4-((2- cyanopyrimidin-4-yl)amino)piperidin-1- yl)methyl)phenyl)-3H-imidazo[4,5- b]pyridine-5-carbonitrile |
| 407 | 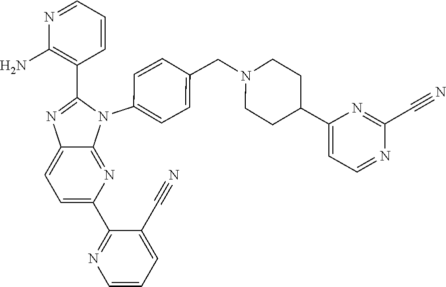 [SMILES: N#Cc1nccc(C2CCN(Cc3ccc(-n4c(-c5cccnc5N)nc5ccc(-c6ncccc6C#N)nc54)cc3)CC2)n1] | 4-(4-(4-(2-(2-aminopyridin-3-yl)-5-(3- cyanopyridin-2-yl)-3H-imidazo[4,5- b]pyridin-3-yl)benzyl)piperazin-1- yl)pyrimidine-2-carbonitrile |
| 408 | 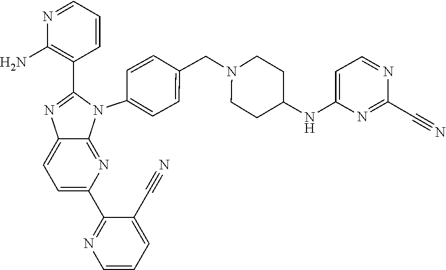 [SMILES: N#Cc1nccc(NC2CCN(Cc3ccc(-n4c(-c5cccnc5N)nc5ccc(-c6ncccc6C#N)nc54)cc3)CC2)n1] | 4-((1-(4-(2-(2-aminopyridin-3-yl)-5-(3- cyanopyridin-2-yl)-3H-imidazo[4,5- b]pyridin-3-yl)benzyl)piperidin-4- yl)amino)pyrimidine-2-carbonitrile |
| 409 | 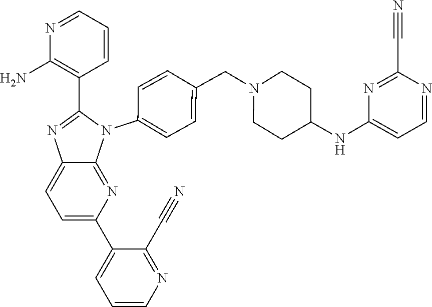 [SMILES: N#Cc1nccc(NC2CCN(Cc3ccc(-n4c(-c5cccnc5N)nc5ccc(-c6cccnc6C#N)nc54)cc3)CC2)n1] | 4-((1-(4-(2-(2-aminopyridin-3-yl)-5-(2- cyanopyridin-3-yl)-3H-imidazo[4,5- b]pyridin-3-yl)benzyl)piperidin-4- yl)amino)pyrimidine-2-carbonitrile |
| 410 | 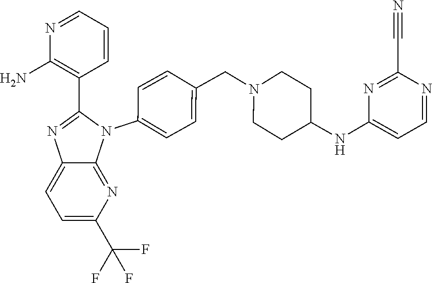 [SMILES: N#Cc1nccc(NC2CCN(Cc3ccc(-n4c(-c5cccnc5N)nc5ccc(C(F)(F)F)nc54)cc3)CC2)n1] | 4-((1-(4-(2-(2-aminopyridin-3-yl)-5- (trifluoromethyl)-3H-imidazo[4,5- b]pyridin-3-yl)benzyl)piperidin-4- yl)amino)pyrimidine-2-carbonitrile |
| 411 | 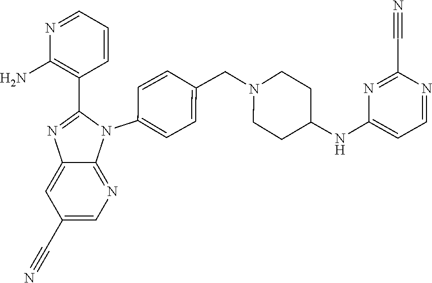 [SMILES: N#Cc1cnc2c(c1)nc(-c1cccnc1N)n2-c1ccc(CN2CCC(Nc3ccnc(C#N)n3)CC2)cc1] | 2-(2-aminopyridin-3-yl)-3-(4-((4-((2- cyanopyrimidin-4-yl)amino)piperidin-1- yl)methyl)phenyl)-3H-imidazo[4,5- b]pyridine-6-carbonitrile |
| 412 | 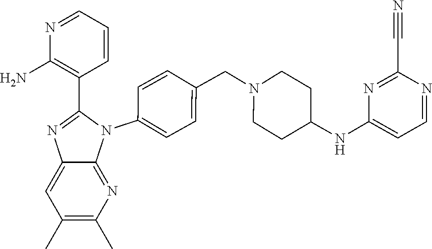 [SMILES: Cc1cc2nc(-c3cccnc3N)n(-c3ccc(CN4CCC(Nc5ccnc(C#N)n5)CC4)cc3)c2nc1C] | 4-((1-(4-(2-(2-aminopyridin-3-yl)-5,6- dimethyl-3H-imidazo[4,5-b]pyridin-3- yl)benzyl)piperidin-4-yl)amino)pyrimidine- 2-carbonitrile |
| 413 | 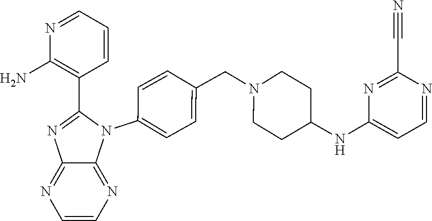 [SMILES: N#Cc1nccc(NC2CCN(Cc3ccc(-n4c(-c5cccnc5N)nc5nccnc54)cc3)CC2)n1] | 4-((1-(4-(2-(2-aminopyridin-3-yl)-1H- imidazo[4,5-b]pyrazin-1- yl)benzyl)piperidin-4-yl)amino)pyrimidine- 2-carbonitrile |
| 414 | 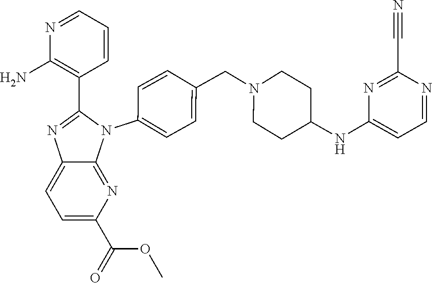 [SMILES: COC(=O)c1ccc2nc(-c3cccnc3N)n(-c3ccc(CN4CCC(Nc5ccnc(C#N)n5)CC4)cc3)c2n1] | methyl 2-(2-aminopyridin-3-yl)-3-(4-((4- ((2-cyanopyrimidin-4-yl)amino)piperidin-1- yl)methyl)phenyl)-3H-imidazo[4,5- b]pyridine-S-carboxylate |
| 415 | 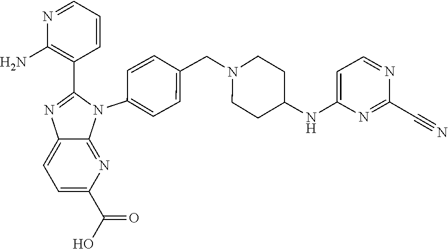 [SMILES: N#Cc1nccc(NC2CCN(Cc3ccc(-n4c(-c5cccnc5N)nc5ccc(C(=O)O)nc54)cc3)CC2)n1] | 2-(2-aminopyridin-3-yl)-3-(4-((4-((2- cyanopyrimidin-4-yl)amino)piperidin-1- yl)methyl)phenyl)-3H-imidazo[4,5- b]pyridine-5-carboxylic acid |
| 416 | 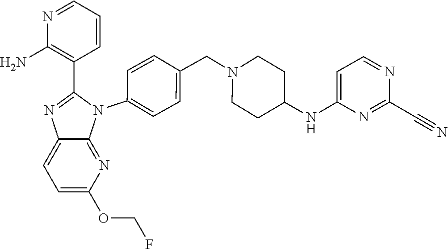 [SMILES: N#Cc1nccc(NC2CCN(Cc3ccc(-n4c(-c5cccnc5N)nc5ccc(OCF)nc54)cc3)CC2)n1] | 4-((1-(4-(2-(2-aminopyridin-3-yl)-5- (fluoromethoxy)-3H-imidazo[4,5-b]pyridin- 3-yl)benzyl)piperidin-4- yl)amino)pyrimidine-2-carbonitrile |
| 417 | 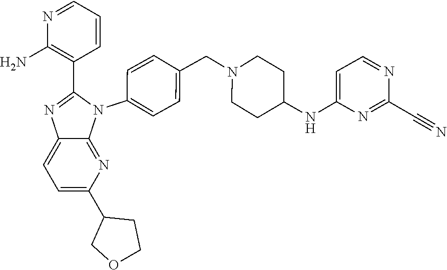 [SMILES: N#Cc1nccc(NC2CCN(Cc3ccc(-n4c(-c5cccnc5N)nc5ccc(C6CCOC6)nc54)cc3)CC2)n1] | 4-((1-(4-(2-(2-aminopyridin-3-yl)-5- (tetrahydrofuran-3-yl)-3H-imidazo[4,5- b]pyridin-3-yl)benzyl)piperidin-4- yl)amino)pyrimidine-2-carbonitrile |
| 418 | 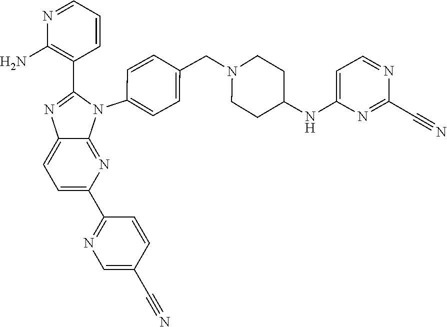 [SMILES: N#Cc1ccc(-c2ccc3nc(-c4cccnc4N)n(-c4ccc(CN5CCC(Nc6ccnc(C#N)n6)CC5)cc4)c3n2)nc1] | 4-((1-(4-(2-(2-aminopyridin-3-yl)-5-(5- cyanopyridin-2-yl)-3H-imidazo[4,5- b]pyridin-3-yl)benzyl)piperidin-4- yl)amino)pyrimidine-2-carbonitrile |
| 419 | 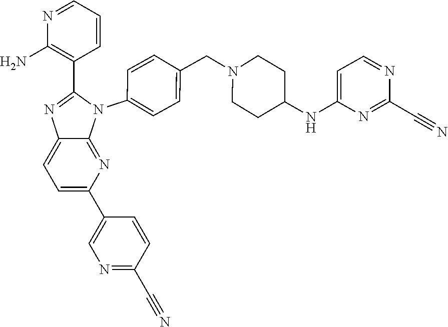 [SMILES: N#Cc1ccc(-c2ccc3nc(-c4cccnc4N)n(-c4ccc(CN5CCC(Nc6ccnc(C#N)n6)CC5)cc4)c3n2)cn1] | 4-((1-(4-(2-(2-aminopyridin-3-yl)-5-(6- cyanopyridin-3-yl)-3H-imidazo[4,5- b]pyridin-3-yl)benzyl)piperidin-4- yl)amino)pyrimidine-2-carbonitrile |
| 420 | 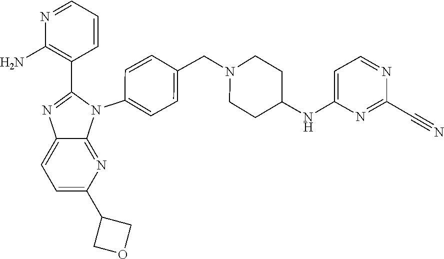 [SMILES: N#Cc1nccc(NC2CCN(Cc3ccc(-n4c(-c5cccnc5N)nc5ccc(C6COC6)nc54)cc3)CC2)n1] | 4-((1-(4-(2-(2-aminopyridin-3-yl)-5- (oxetan-3-yl)-3H-imidazo[4,5-b]pyridin-3- yl)benzyl)piperidin-4-yl)amino)pyrimidine- 2-carbonitrile |
| 421 | 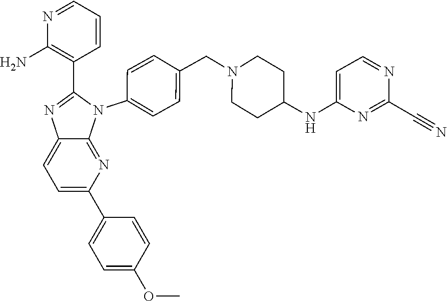 [SMILES: COc1ccc(-c2ccc3nc(-c4cccnc4N)n(-c4ccc(CN5CCC(Nc6ccnc(C#N)n6)CC5)cc4)c3n2)cc1] | 4-((1-(4-(2-(2-aminopyridin-3-yl)-5-(4- methoxyphenyl)-3H-imidazo[4,5-b ]pyridin- 3-yl)benzyl)piperidin-4- yl)amino)pyrimidine-2-carbonitrile |
| 422 | 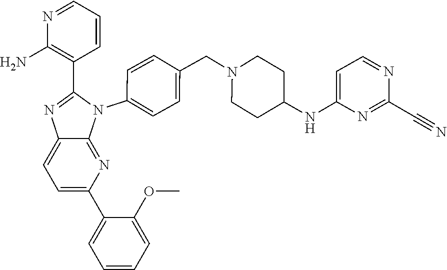 [SMILES: COc1ccccc1-c1ccc2nc(-c3cccnc3N)n(-c3ccc(CN4CCC(Nc5ccnc(C#N)n5)CC4)cc3)c2n1] | 4-((1-(4- (2-(2-aminopyridin-3-yl)-5-(2- methoxyphenyl)-3H-imidazo[4,5-b]pyridin- 3-yl)benzyl)piperidin-4- yl)amino)pyrimidine-2-carbonitrile |
| 423 | 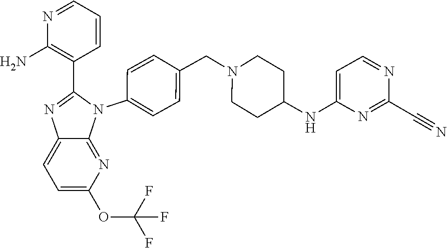 [SMILES: N#Cc1nccc(NC2CCN(Cc3ccc(-n4c(-c5cccnc5N)nc5ccc(OC(F)(F)F)nc54)cc3)CC2)n1] | 4-((1-(4-(2-(2-aminopyridin-3-yl)-5- (trifluoromethoxy)-3H-imidazo[4,5- b]pyridin-3-yl)benzyl)piperidin-4- yl)amino)pyrimidine-2-carbonitrile |
| 424 | 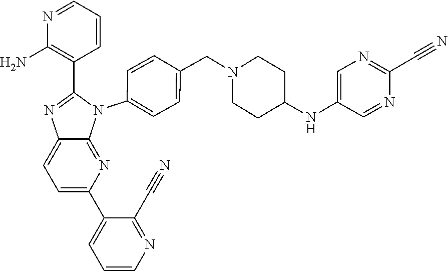 [SMILES: N#Cc1ncc(NC2CCN(Cc3ccc(-n4c(-c5cccnc5N)nc5ccc(-c6cccnc6C#N)nc54)cc3)CC2)cn1] | 4-(4-(4-(2-(2-aminopyridin-3-yl)-5-(2- cyanopyridin-3-yl)-3H-imidazo[4,5- b]pyridin-3-yl)benzyl)piperazin-1- yl)pyrimidine-2-carbonitrile |
| 425 | 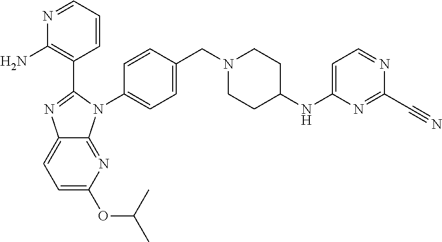 [SMILES: CC(C)Oc1ccc2nc(-c3cccnc3N)n(-c3ccc(CN4CCC(Nc5ccnc(C#N)n5)CC4)cc3)c2n1] | 4-((1-(4-(2-(2-aminopyridin-3-yl)-5- isopropoxy-3H-imidazo[4,5-b]pyridin-3- yl)benzyl)piperidin-4-yl)amino)pyrimidine- 2-carbonitrile |
| 426 | 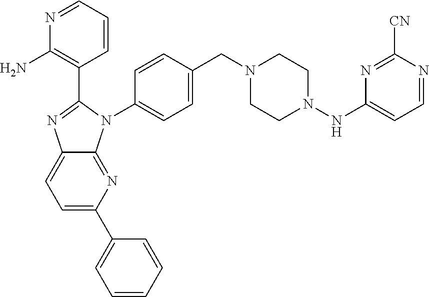 [SMILES: N#Cc1nccc(NN2CCN(Cc3ccc(-n4c(-c5cccnc5N)nc5ccc(-c6ccccc6)nc54)cc3)CC2)n1] | 4-((4-(4-(2-(2-aminopyridin-3-yl)-5-phenyl- 3H-imidazo[4,5-b]pyridin-3- yl)benzyl)piperazin-1-yl)amino)pyrimidine- 2-carbonitrile |
| 427 | 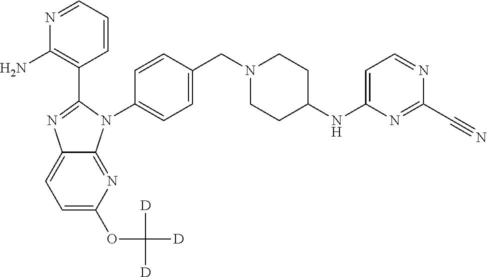 [SMILES: [2H]C([2H])([2H])Oc1ccc2nc(-c3cccnc3N)n(-c3ccc(CN4CCC(Nc5ccnc(C#N)n5)CC4)cc3)c2n1] | 4-((1-(4-(2-(2-aminopyridin-3-yl)-5- (methoxy-d3)-3H-imidazo[4,5-b]pyridin-3- yl)benzyl)piperidin-4-yl)amino)pyrimidine- 2-carbonitrile |
| 428 | 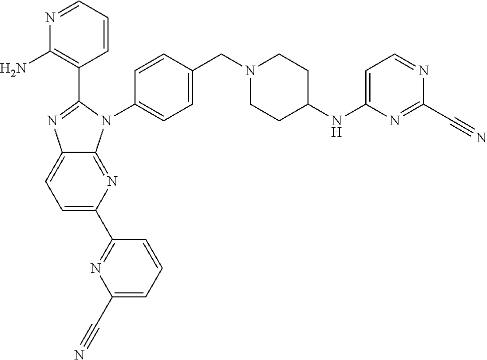 [SMILES: N#Cc1cccc(-c2ccc3nc(-c4cccnc4N)n(-c4ccc(CN5CCC(Nc6ccnc(C#N)n6)CC5)cc4)c3n2)n1] | 4-((1-(4-(2-(2-aminopyridin-3-yl)-5-(6- cyanopyridin-2-yl)-3H-imidazo[4,5- b]pyridin-3-yl)benzyl)piperidin-4- yl)amino)pyrimidine-2-carbonitrile |
| 429 | 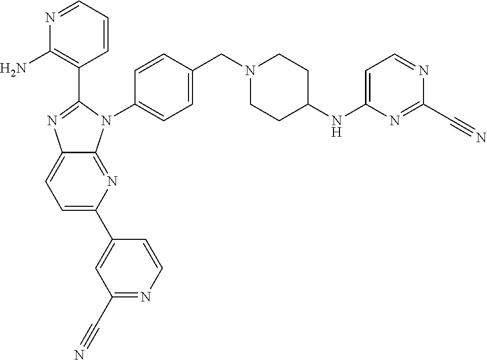 [SMILES: N#Cc1cc(-c2ccc3nc(-c4cccnc4N)n(-c4ccc(CN5CCC(Nc6ccnc(C#N)n6)CC5)cc4)c3n2)ccn1] | 4-((1-(4-(2-(2-aminopyridin-3-yl)-5-(2- cyanopyridin-4-yl)-3H-imidazo[4,5- b]pyridin-3-yl)benzyl)piperidin-4- yl)amino)pyrimidine-2-carbonitrile |
| 430 | 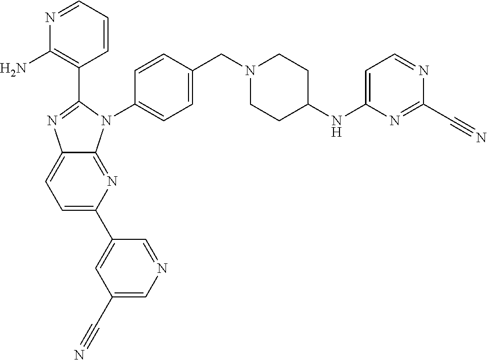 [SMILES: N#Cc1cncc(-c2ccc3nc(-c4cccnc4N)n(-c4ccc(CN5CCC(Nc6ccnc(C#N)n6)CC5)cc4)c3n2)c1] | 4-((1-(4-(2-(2-aminopyridin-3-yl)-5-(5- cyanopyridin-3-yl)-3H-imidazo[4,5- b]pyridin-3-yl)benzyl)piperidin-4- yl)amino)pyrimidine-2-carbonitrile |
| 431 | 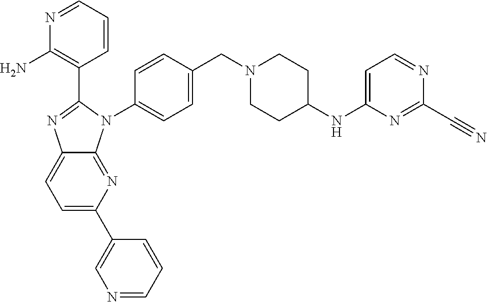 [SMILES: N#Cc1nccc(NC2CCN(Cc3ccc(-n4c(-c5cccnc5N)nc5ccc(-c6cccnc6)nc54)cc3)CC2)n1] | 4-((1-(4-(2-(2-aminopyridin-3-yl)-5- (pyridin-3-yl)-3H-imidazo[4,5-b]pyridin-3- yl)benzyl)piperidin-4-yl)amino)pyrimidine- 2-carbonitrile |
| 432 | 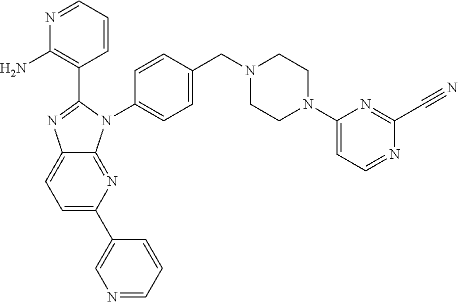 [SMILES: N#Cc1nccc(N2CCN(Cc3ccc(-n4c(-c5cccnc5N)nc5ccc(-c6cccnc6)nc54)cc3)CC2)n1] | 4-(4-(4-(2-(2-aminopyridin-3-yl)-5- (pyridin-3-yl)-3H-imidazo[4,5-b]pyridin-3- yl)benzyl)piperazin-1-yl)pyrimidine-2- carbonitrile |
| 433 | 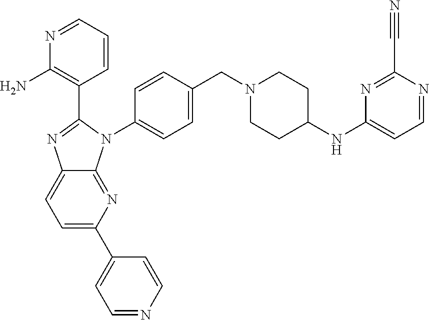 [SMILES: N#Cc1nccc(NC2CCN(Cc3ccc(-n4c(-c5cccnc5N)nc5ccc(-c6ccncc6)nc54)cc3)CC2)n1] | 4-((1-(4-(2-(2-aminopyridin-3-yl)-5- (pyridin-4-yl)-3H-imidazo[4,5-b]pyridin-3- yl)benzyl)piperidin-4-yl)amino)pyrimidine- 2-carbonitrile |
| 434 | 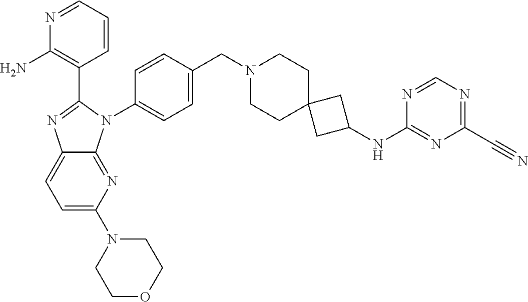 [SMILES: N#Cc1ncnc(NC2CC3(CCN(Cc4ccc(-n5c(-c6cccnc6N)nc6ccc(N7CCOCC7)nc65)cc4)CC3)C2)n1] | 4-((7-(4-(2-(2-aminopyridin-3-yl)-5- morpholino-3H-imidazo[4,5-b]pyridin-3- yl)benzyl)-7-azaspiro[3.5]nonan-2- yl)amino)-1,3,5-triazine-2-carbonitrile |
| 435 | 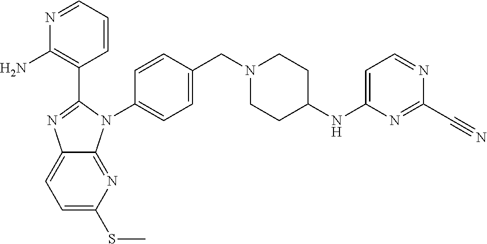 [SMILES: CSc1ccc2nc(-c3cccnc3N)n(-c3ccc(CN4CCC(Nc5ccnc(C#N)n5)CC4)cc3)c2n1] | 4-((1-(4-(2-(2-aminopyridin-3-yl)-5- (methylthio)-3H-imidazo[4,5-b]pyridin-3- yl)benzyl)piperidin-4-yl)amino)pyrimidine- 2-carbonitrile |
| 436 | 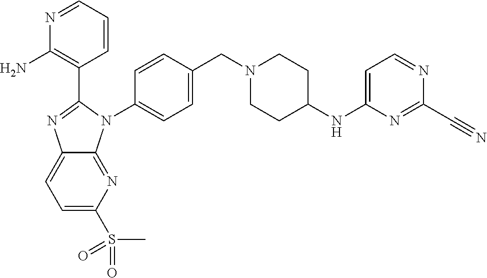 [SMILES: CS(=O)(=O)c1ccc2nc(-c3cccnc3N)n(-c3ccc(CN4CCC(Nc5ccnc(C#N)n5)CC4)cc3)c2n1] | 4-((1-(4-(2-(2-aminopyridin-3-yl)-5- (methylsulfonyl)-3H-imidazo[4,5-b ]pyridin- 3-yl)benzyl)piperidin-4- yl)amino)pyrimidine-2-carbonitrile |
| 437 | 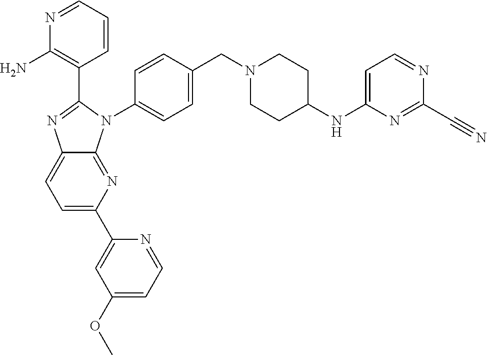 [SMILES: COc1ccnc(-c2ccc3nc(-c4cccnc4N)n(-c4ccc(CN5CCC(Nc6ccnc(C#N)n6)CC5)cc4)c3n2)c1] | 4-((1-(4-(2-(2-aminopyridin-3-yl)-5-(4- methoxypyridin-2-yl)-3H-imidazo[4,5- b]pyridin-3-yl)benzyl)piperidin-4- yl)amino)pyrimidine-2-carbonitrile |
| 438 | 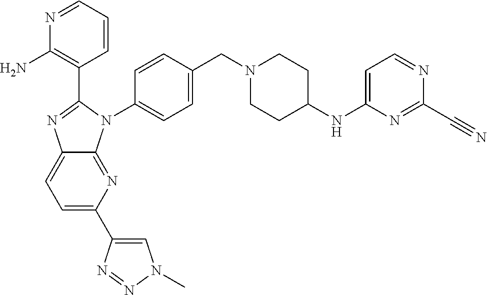 [SMILES: Cn1cc(-c2ccc3nc(-c4cccnc4N)n(-c4ccc(CN5CCC(Nc6ccnc(C#N)n6)CC5)cc4)c3n2)nn1] | 4-((1-(4-(2-(2-aminopyridin-3-yl)-5-(1- methyl-1H-1,2,3-triazol-4-yl)-3H- imidazo[4,5-b]pyridin-3- yl)benzyl)piperidin-4-yl)amino)pyrimidine- 2-carbonitrile |
| 439 | 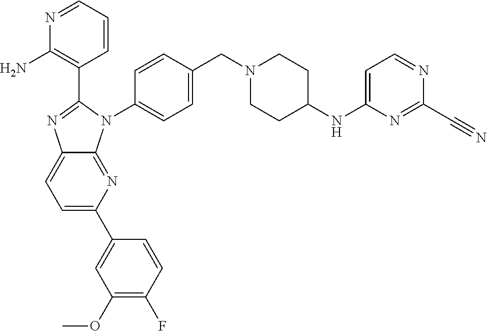 [SMILES: COc1cc(-c2ccc3nc(-c4cccnc4N)n(-c4ccc(CN5CCC(Nc6ccnc(C#N)n6)CC5)cc4)c3n2)ccc1F] | 4-((1-(4-(2-(2-aminopyridin-3-yl)-5-(4- fluoro-3-methoxyphenyl)-3H-imidazo[4,5- b]pyridin-3-yl)benzyl)piperidin-4- yl)amino)pyrimidine-2-carbonitrile |
| 440 | 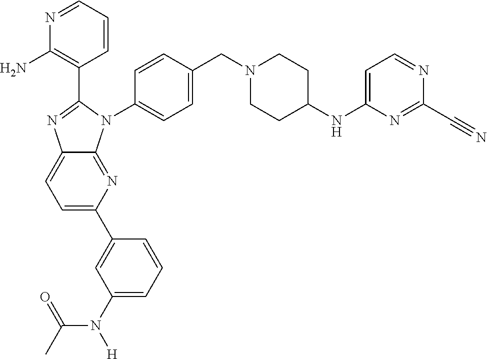 [SMILES: CC(=O)Nc1cccc(-c2ccc3nc(-c4cccnc4N)n(-c4ccc(CN5CCC(Nc6ccnc(C#N)n6)CC5)cc4)c3n2)c1] | N-(3-(2-(2-aminopyridin-3-yl)-3-(4-((4-((2- cyanopyrimidin-4-yl)amino)piperidin-1- yl)methyl)phenyl)-3H-imidazo[4,5- b]pyridin-5-yl)phenyl)acetamide |
| 441 | 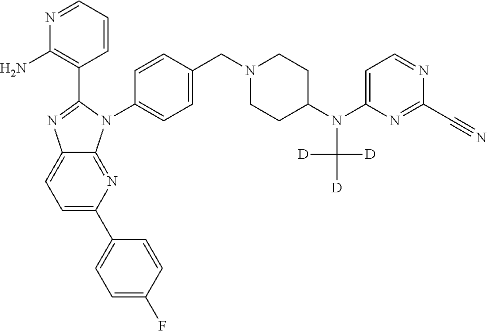 [SMILES: [2H]C([2H])([2H])N(c1ccnc(C#N)n1)C1CCN(Cc2ccc(-n3c(-c4cccnc4N)nc4ccc(-c5ccc(F)cc5)nc43)cc2)CC1] | 4-((1-(4-(2-(2-aminopyridin-3-yl)-5-(4- fluorophenyl)-3H-imidazo[4,5-b]pyridin-3- yl)benzyl)piperidin-4-yl)(methyl- d3)amino)pyrimidine-2-carbonitrile |
| 442 | 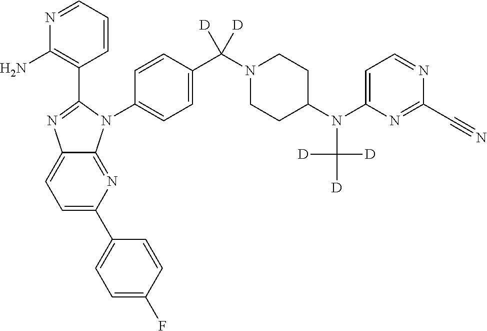 [SMILES: [2H]C([2H])([2H])N(c1ccnc(C#N)n1)C1CCN(C([2H])([2H])c2ccc(-n3c(-c4cccnc4N)nc4ccc(-c5ccc(F)cc5)nc43)cc2)CC1] | 4-((1-((4-(2-(2-aminopyridin-3-yl)-5-(4- fluorophenyl)-3H-imidazo[4,5-b]pyridin-3- yl)phenyl)methyl-d2)piperidin-4- yl)(methyl-d3)amino)pyrimidine-2- carbonitrile |
| 443 | 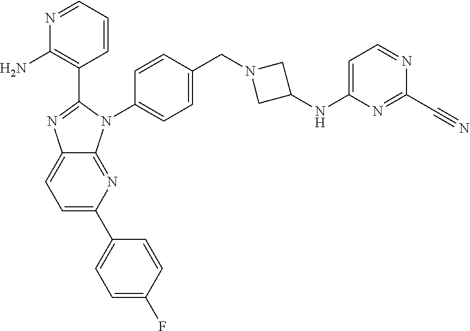 [SMILES: N#Cc1nccc(NC2CN(Cc3ccc(-n4c(-c5cccnc5N)nc5ccc(-c6ccc(F)cc6)nc54)cc3)C2)n1] | 4-((1-(4-(2-(2-aminopyridin-3-yl)-5-(4- fluorophenyl)-3H-imidazo[4,5-b]pyridin-3- yl)benzyl)azetidin-3-yl)amino)pyrimidine- 2-carbonitrile |
| 444 | 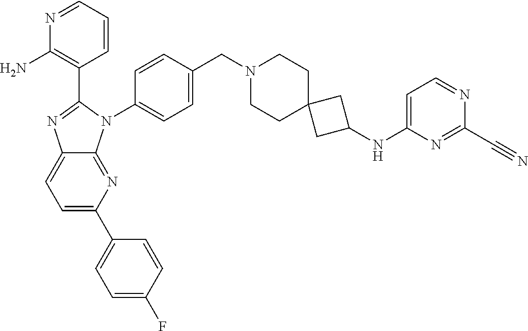 [SMILES: N#Cc1nccc(NC2CC3(CCN(Cc4ccc(-n5c(-c6cccnc6N)nc6ccc(-c7ccc(F)cc7)nc65)cc4)CC3)C2)n1] | 4-((7-(4-(2-(2-aminopyridin-3-yl)-5-(4- fluorophenyl)-3H-imidazo[4,5-b]pyridin-3- yl)benzyl)-7-azaspiro[3.5]nonan-2- yl)amino)pyrimidine-2-carbonitrile |
| 445 | 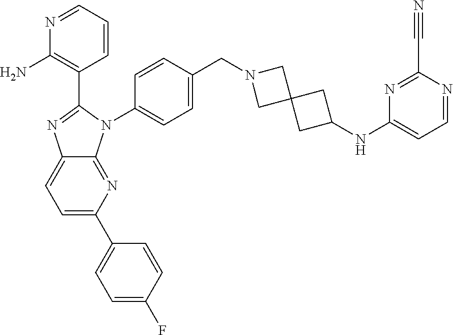 [SMILES: N#Cc1nccc(NC2CC3(C2)CN(Cc2ccc(-n4c(-c5cccnc5N)nc5ccc(-c6ccc(F)cc6)nc54)cc2)C3)n1] | 4-((2-(4-(2-(2-aminopyridin-3-yl)-5-(4- fluorophenyl)-3H-imidazo[4,5-b]pyridin-3- yl)benzyl)-2-azaspiro[3.3 ]heptan-6- yl)amino)pyrimidine-2-carbonitrile |
| 446 | 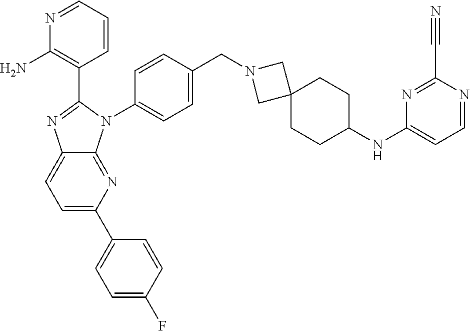 [SMILES: N#Cc1nccc(NC2CCC3(CC2)CN(Cc2ccc(-n4c(-c5cccnc5N)nc5ccc(-c6ccc(F)cc6)nc54)cc2)C3)n1] | 4-((2-(4-(2-(2-aminopyridin-3-yl)-5-(4- fluorophenyl)-3H-imidazo[4,5-b]pyridin-3- yl)benzyl)-2-azaspiro[3.5]nonan-7- yl)amino)pyrimidine-2-carbonitrile |
| 447 | 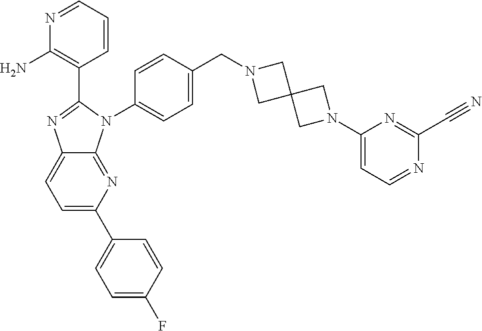 [SMILES: N#Cc1nccc(N2CC3(CN(Cc4ccc(-n5c(-c6cccnc6N)nc6ccc(-c7ccc(F)cc7)nc65)cc4)C3)C2)n1] | 4-(6-(4-(2-(2-aminopyridin-3-yl)-5-(4- fluorophenyl)-3H-imidazo[4,5-b]pyridin-3- yl)benzyl)-2,6-diazaspiro[3.3]heptan-2- yl)pyrimidine-2-carbonitrile |
| 448 | 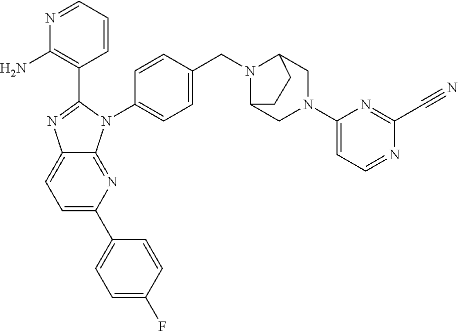 [SMILES: N#Cc1nccc(N2CC3CCC(C2)N3Cc2ccc(-n3c(-c4cccnc4N)nc4ccc(-c5ccc(F)cc5)nc43)cc2)n1] | 4-(8-(4-(2-(2-aminopyridin-3-yl)-5-(4- fluorophenyl)-3H-imidazo[4,5-b]pyridin-3- yl)benzyl)-3,8-diazabicyclo[3.2.1]octan-3- yl)pyrimidine-2-carbonitrile |
| 449 | 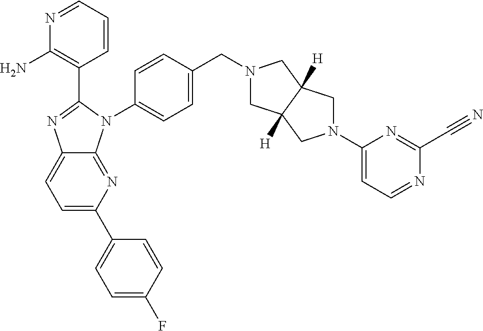 [SMILES: N#Cc1nccc(N2C[C@H]3CN(Cc4ccc(-n5c(-c6cccnc6N)nc6ccc(-c7ccc(F)cc7)nc65)cc4)C[C@H]3C2)n1] | 4-((3aR,6aS)-5-(4-(2-(2-aminopyridin- 3- yl)-5-(4-fluorophenyl)-3H-imidazo[4,5- b]pyridin-3- yl)benzyl)hexahydropyrrolo[3,4-c]pyrrol- 2(1H)-yl)pyrimidine-2-carbonitrile |
| 450 | 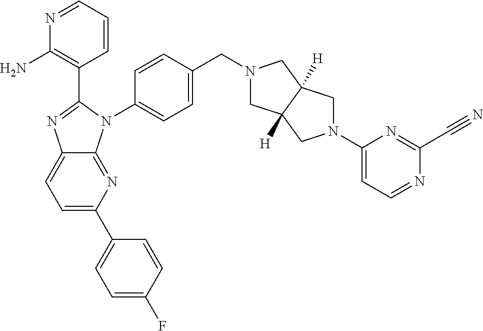 [SMILES: N#Cc1nccc(N2C[C@@H]3CN(Cc4ccc(-n5c(-c6cccnc6N)nc6ccc(-c7ccc(F)cc7)nc65)cc4)C[C@H]3C2)n1] | 4-((3aS,6aS)-5-(4-(2-(2-aminopyridin-3-yl)- 5-(4-fluorophenyl)-3H-imidazo[4,5- b]pyridin-3- yl)benzyl)hexahydropyrrolo[3,4-c]pyrrol- 2(1H)-yl)pyrimidine-2-carbonitrile |
| 451 | 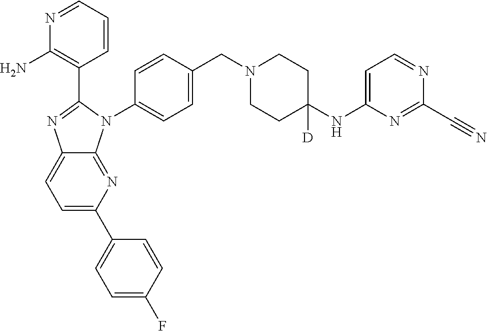 [SMILES: [2H]C1(Nc2ccnc(C#N)n2)CCN(Cc2ccc(-n3c(-c4cccnc4N)nc4ccc(-c5ccc(F)cc5)nc43)cc2)CC1] | 4-((1-(4-(2-(2-aminopyridin-3-yl)-5-(4- fluorophenyl)-3H-imidazo[4,5-b]pyridin-3- yl)benzyl)piperidin-4-yl-4- d)amino)pyrimidine-2-carbonitrile |
| 452 | 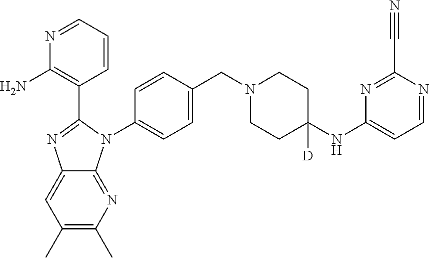 [SMILES: [2H]C1(Nc2ccnc(C#N)n2)CCN(Cc2ccc(-n3c(-c4cccnc4N)nc4cc(C)c(C)nc43)cc2)CC1] | 4-((1-(4-(2-(2-aminopyridin-3-yl)-5,6- dimethyl-3H-imidazo[4,5-b]pyridin-3- yl)benzyl)piperidin-4-yl-4- d)amino)pyrimidine-2-carbonitrile |
| 453 | 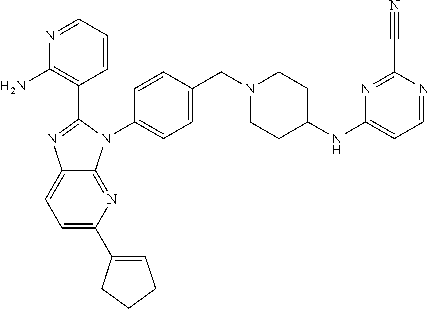 [SMILES: N#Cc1nccc(NC2CCN(Cc3ccc(-n4c(-c5cccnc5N)nc5ccc(C6=CCCC6)nc54)cc3)CC2)n1] | 4-((1-(4-(2-(2-aminopyridin-3-yl)-5- (cyclopent-1-en-1-yl)-3H-imidazo[4,5- b]pyridin-3-yl)benzyl)piperidin-4- yl)amino)pyrimidine-2-carbonitrile |
| 454 | 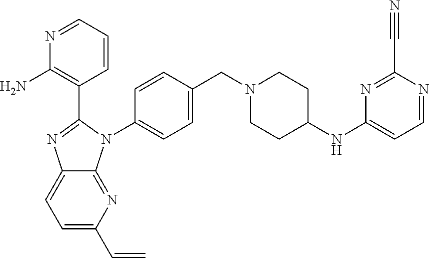 [SMILES: C=Cc1ccc2nc(-c3cccnc3N)n(-c3ccc(CN4CCC(Nc5ccnc(C#N)n5)CC4)cc3)c2n1] | 4-((1-(4-(2-(2-aminopyridin-3-yl)-5-vinyl- 3H-imidazo[4,5-b ]pyridin-3- yl)benzyl)piperidin-4-yl)amino)pyrimidine- 2-carbonitrile |
| 455 | 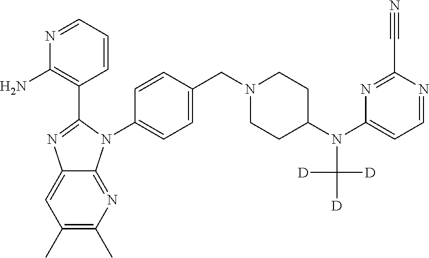 [SMILES: [2H]C([2H])([2H])N(c1ccnc(C#N)n1)C1CCN(Cc2ccc(-n3c(-c4cccnc4N)nc4cc(C)c(C)nc43)cc2)CC1] | 4-((1-(4-(2-(2-aminopyridin-3-yl)-5,6- dimethyl-3H-imidazo[4,5-b]pyridin-3- yl)benzyl)piperidin-4-yl)(methyl- d3)amino)pyrimidine-2-carbonitrile |
| 456 | 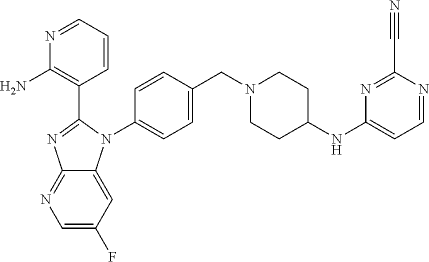 [SMILES: N#Cc1nccc(NC2CCN(Cc3ccc(-n4c(-c5cccnc5N)nc5ncc(F)cc54)cc3)CC2)n1] | 4-((1-(4-(2-(2-aminopyridin-3-yl)-5-(5- fluoropyridin-2-yl)-3H-imidazo[4,5- b]pyridin-3-yl)benzyl)piperidin-4- yl)amino)pyrimidine-2-carbonitrile |
| 457 | 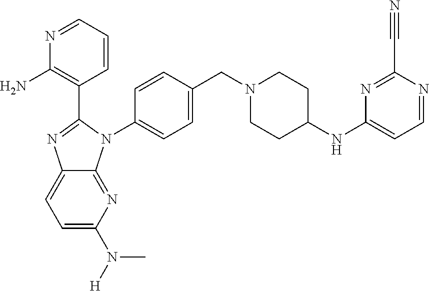 [SMILES: CNc1ccc2nc(-c3cccnc3N)n(-c3ccc(CN4CCC(Nc5ccnc(C#N)n5)CC4)cc3)c2n1] | 4-((1-(4-(2-(2-aminopyridin-3-yl)-5- (methylamino)-3H-imidazo[4,5-b]pyridin- 3-yl)benzyl)piperidin-4- yl)amino)pyrimidine-2-carbonitrile |
| 458 | 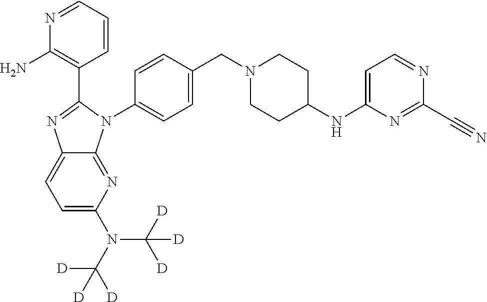 [SMILES: [2H]C([2H])([2H])N(c1ccc2nc(-c3cccnc3N)n(-c3ccc(CN4CCC(Nc5ccnc(C#N)n5)CC4)cc3)c2n1)C([2H])([2H])[2H]] | 4-((1-(4-(2-(2-aminopyridin-3-yl)-5- (bis(methyl-d3)amino)-3H-imidazo[4,5- b]pyridin-3-yl)benzyl)piperidin-4- yl)amino)pyrimidine-2-carbonitrile |
| 459 | 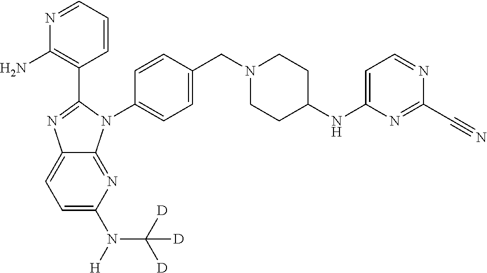 [SMILES: [2H]C([2H])([2H])Nc1ccc2nc(-c3cccnc3N)n(-c3ccc(CN4CCC(Nc5ccnc(C#N)n5)CC4)cc3)c2n1] | 4-((1-(4-(2-(2-aminopyridin-3-yl)-5- ((methyl-d3)amino)-3H-imidazo[4,5- b]pyridin-3-yl)benzyl)piperidin-4- yl)amino)pyrimidine-2-carbonitrile |
| 460 | 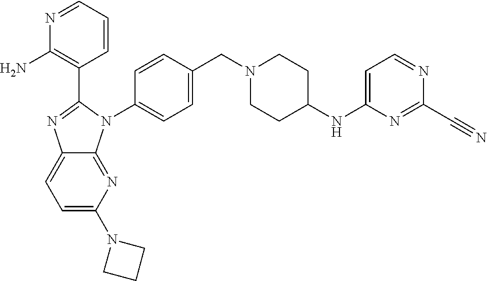 [SMILES: N#Cc1nccc(NC2CCN(Cc3ccc(-n4c(-c5cccnc5N)nc5ccc(N6CCC6)nc54)cc3)CC2)n1] | 4-((1-(4-(2-(2-aminopyridin-3-yl)-5- (azetidin-1-yl)-3H-imidazo[4,5-b]pyridin-3- yl)benzyl)piperidin-4-yl)amino)pyrimidine- 2-carbonitrile |
| 461 | 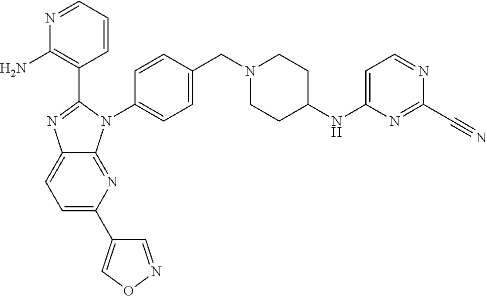 [SMILES: N#Cc1nccc(NC2CCN(Cc3ccc(-n4c(-c5cccnc5N)nc5ccc(-c6cnoc6)nc54)cc3)CC2)n1] | 4-((1-(4-(2-(2-aminopyridin-3-yl)-5- (isoxazol-4-yl)-3H-imidazo[4,5-b]pyridin- 3-yl)benzyl)piperidin-4- yl)amino)pyrimidine-2-carbonitrile |
| 462 | 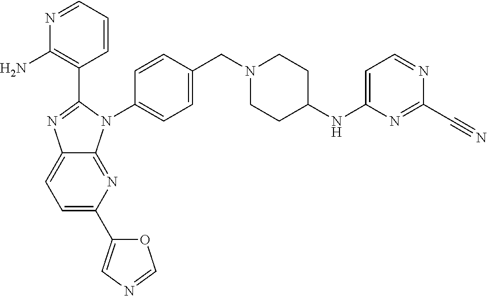 [SMILES: N#Cc1nccc(NC2CCN(Cc3ccc(-n4c(-c5cccnc5N)nc5ccc(-c6cnco6)nc54)cc3)CC2)n1] | 4-((1-(4-(2-(2-aminopyridin-3-yl)-5- (oxazol-5-yl)-3H-imidazo[4,5-b]pyridin-3- yl)benzyl)piperidin-4-yl)amino)pyrimidine- 2-carbonitrile |
| 463 | 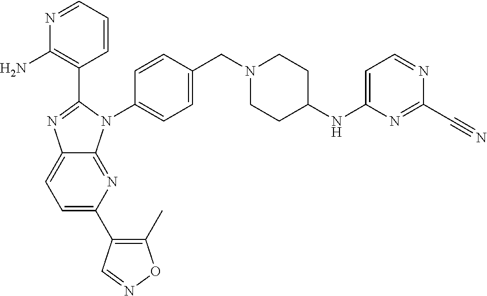 [SMILES: Cc1oncc1-c1ccc2nc(-c3cccnc3N)n(-c3ccc(CN4CCC(Nc5ccnc(C#N)n5)CC4)cc3)c2n1] | 4-((1-(4-(2-(2-aminopyridin-3-yl)-5-(5- methylisoxazol-4-yl)-3H-imidazo[4,5- b]pyridin-3-yl)benzyl)piperidin-4- yl)amino)pyrimidine-2-carbonitrile |
| 464 | 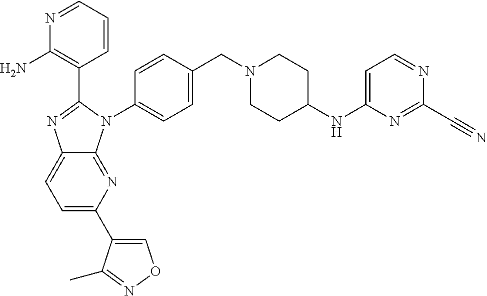 [SMILES: Cc1nocc1-c1ccc2nc(-c3cccnc3N)n(-c3ccc(CN4CCC(Nc5ccnc(C#N)n5)CC4)cc3)c2n1] | 4-((1-(4-(2-(2-aminopyridin-3-yl)-5-(3- methylisoxazol-4-yl)-3H-imidazo[4,5- b]pyridin-3-yl)benzyl)piperidin-4- yl)amino)pyrimidine-2-carbonitrile |
| 465 | 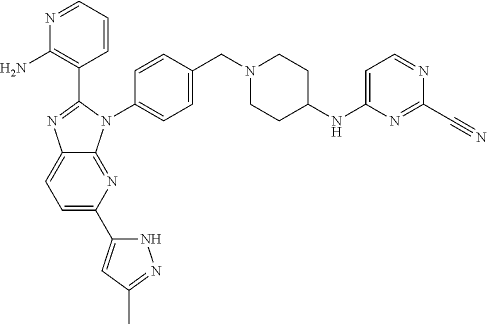 [SMILES: Cc1cc(-c2ccc3nc(-c4cccnc4N)n(-c4ccc(CN5CCC(Nc6ccnc(C#N)n6)CC5)cc4)c3n2)[nH]n1] | 4-((1-(4-(2-(2-aminopyridin-3-yl)-5-(3- methyl-1H-pyrazol-5-yl)-3H-imidazo[4,5- b]pyridin-3-yl)benzyl)piperidin-4- yl)amino)pyrimidine-2-carbonitrile |
| 466 | 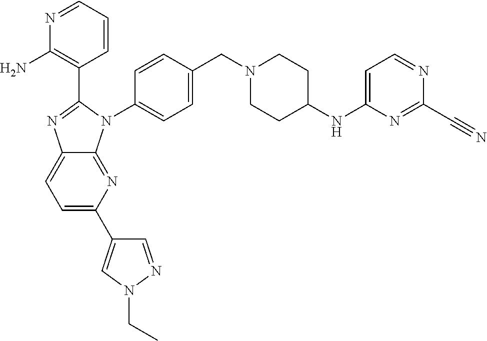 [SMILES: CCn1cc(-c2ccc3nc(-c4cccnc4N)n(-c4ccc(CN5CCC(Nc6ccnc(C#N)n6)CC5)cc4)c3n2)cn1] | 4-((1-(4-(2-(2-aminopyridin-3-yl)-5-(1- ethyl-1H-pyrazol-4-yl)-3H-imidazo[4,5- b]pyridin-3-yl)benzyl)piperidin-4- yl)amino)pyrimidine-2-carbonitrile |
| 467 | 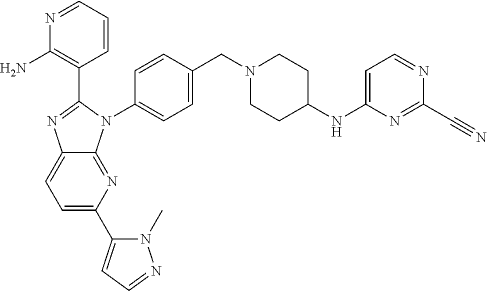 [SMILES: Cn1nccc1-c1ccc2nc(-c3cccnc3N)n(-c3ccc(CN4CCC(Nc5ccnc(C#N)n5)CC4)cc3)c2n1] | 4-((1-(4-(2-(2-aminopyridin-3-yl)-5-(1- methyl-1H-pyrazol-5-yl)-3H-imidazo[4,5- b]pyridin-3-yl)benzyl)piperidin-4- yl)amino)pyrimidine-2-carbonitrile |
| 468 | 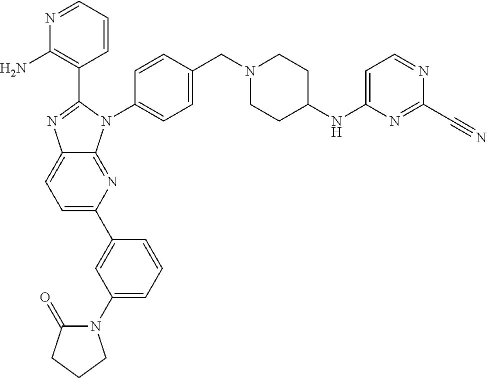 [SMILES: N#Cc1nccc(NC2CCN(Cc3ccc(-n4c(-c5cccnc5N)nc5ccc(-c6cccc(N7CCCC7=O)c6)nc54)cc3)CC2)n1] | 4-((1-(4-(2-(2-aminopyridin-3-yl)-5-(3-(2- oxopyrrolidin-1-yl)phenyl)-3H- imidazo[4,5-b]pyridin-3- yl)benzyl)piperidin-4-yl)amino)pyrimidine- 2-carbonitrile |
| 469 | 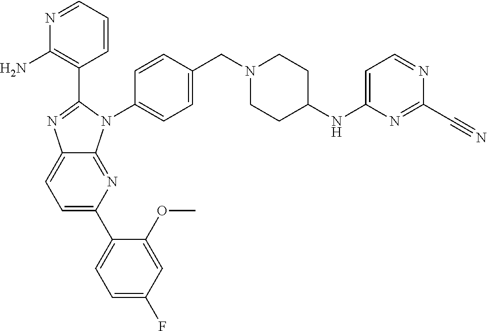 [SMILES: COc1cc(F)ccc1-c1ccc2nc(-c3cccnc3N)n(-c3ccc(CN4CCC(Nc5ccnc(C#N)n5)CC4)cc3)c2n1] | 4-((1-(4-(2-(2-aminopyridin-3-yl)-5-(4- fluoro-2-methoxyphenyl)-3H-imidazo[4,5- b]pyridin-3-yl)benzyl)piperidin-4- yl)amino)pyrimidine-2-carbonitrile |
| 470 | 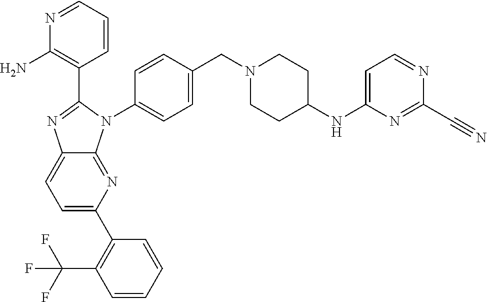 [SMILES: N#Cc1nccc(NC2CCN(Cc3ccc(-n4c(-c5cccnc5N)nc5ccc(-c6ccccc6C(F)(F)F)nc54)cc3)CC2)n1] | 4-((1-(4-(2-(2-aminopyridin-3-yl)-5-(2- (trifluoromethyl)phenyl)-3H-imidazo[4,5- b]pyridin-3-yl)benzyl)piperidin-4- yl)amino)pyrimidine-2-carbonitrile |
| 471 | 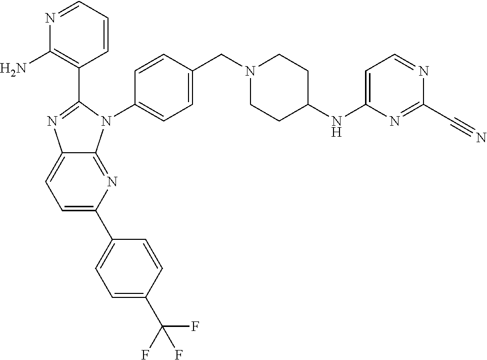 [SMILES: N#Cc1nccc(NC2CCN(Cc3ccc(-n4c(-c5cccnc5N)nc5ccc(-c6ccc(C(F)(F)F)cc6)nc54)cc3)CC2)n1] | 4-((1-(4-(2-(2-aminopyridin-3-yl)-5-(4- (trifluoromethyl)phenyl)-3H-imidazo[4,5- b]pyridin-3-yl)benzyl)piperidin-4- yl)amino)pyrimidine-2-carbonitrile |
| 472 | 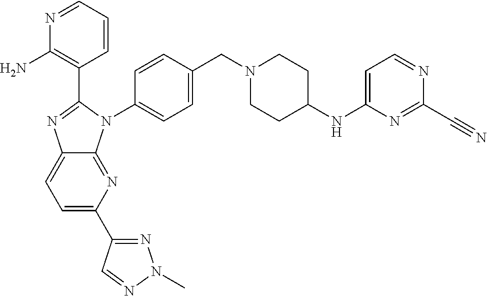 [SMILES: Cn1ncc(-c2ccc3nc(-c4cccnc4N)n(-c4ccc(CN5CCC(Nc6ccnc(C#N)n6)CC5)cc4)c3n2)n1] | 4-((1-(4-(2-(2-aminopyridin-3-yl)-5-(2- methyl-2H-1,2,3-triazol-4-yl)-3H- imidazo[4,5-b ]pyridin-3- yl)benzyl)piperidin-4-yl)amino)pyrimidino- 2-carbonitrile |
| 473 | 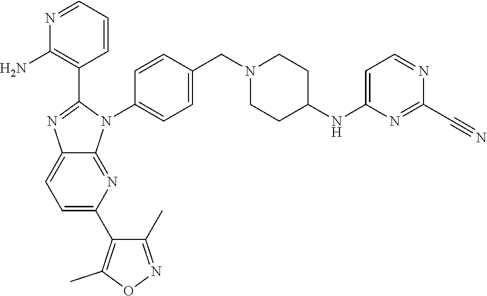 [SMILES: Cc1noc(C)c1-c1ccc2nc(-c3cccnc3N)n(-c3ccc(CN4CCC(Nc5ccnc(C#N)n5)CC4)cc3)c2n1] | 4-((1-(4-(2-(2-aminopyridin-3-yl)-5-(3,5- dimethylisoxazol-4-yl)-3H-imidazo[4,5- b]pyridin-3-yl)benzyl)piperidin-4- yl)amino)pyrimidine-2-carbonitrile |
| 474 | 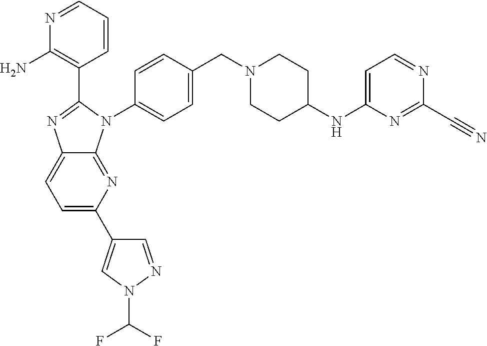 [SMILES: N#Cc1nccc(NC2CCN(Cc3ccc(-n4c(-c5cccnc5N)nc5ccc(-c6cnn(C(F)F)c6)nc54)cc3)CC2)n1] | 4-((1-(4-(2-(2-aminopyridin-3-yl)-5-(1- (difluoromethyl)-1H-pyrazol-4-yl)-3H- imidazo[4,5-b]pyridin-3- yl)benzyl)piperidin-4-yl)amino)pyrimidine- 2-carbonitrile |
| 475 | 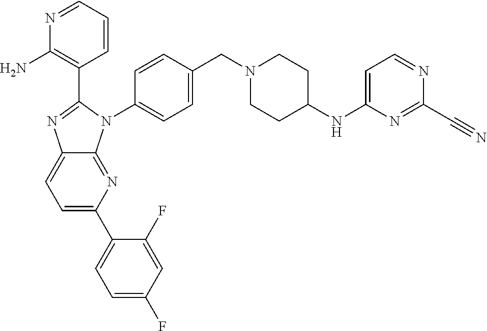 [SMILES: N#Cc1nccc(NC2CCN(Cc3ccc(-n4c(-c5cccnc5N)nc5ccc(-c6ccc(F)cc6F)nc54)cc3)CC2)n1] | 4-((1-(4-(2-(2-aminopyridin-3-yl)-5-(2,4- difluorophenyl)-3H-imidazo[4,5-b]pyridin- 3-yl)benzyl)piperidin-4- yl)amino)pyrimidine-2-carbonitrile |
| 476 | 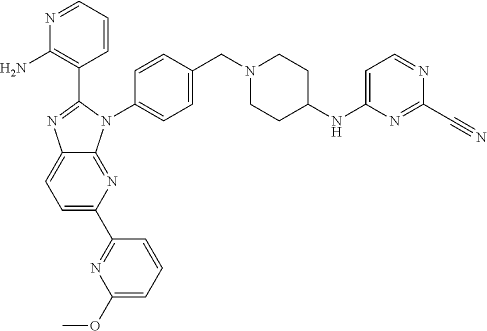 [SMILES: COc1cccc(-c2ccc3nc(-c4cccnc4N)n(-c4ccc(CN5CCC(Nc6ccnc(C#N)n6)CC5)cc4)c3n2)n1] | 4-((1-(4-(2-(2-aminopyridin-3-yl)-5-(6- methoxypyridin-2-yl)-3H-imidazo[4,5- b]pyridin-3-yl)benzyl)piperidin-4- yl)amino)pyrimidine-2-carbonitrile |
| 477 | 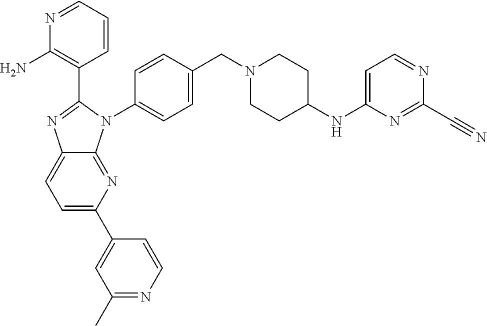 [SMILES: Cc1cc(-c2ccc3nc(-c4cccnc4N)n(-c4ccc(CN5CCC(Nc6ccnc(C#N)n6)CC5)cc4)c3n2)ccn1] | 4-((1-(4-(2-(2-aminopyridin-3-yl)-5-(2- methylpyridin-4-yl)-3H-imidazo[4,5- b]pyridin-3-yl)benzyl)piperidin-4- yl)amino)pyrimidine-2-carbonitrile |
| 478 | 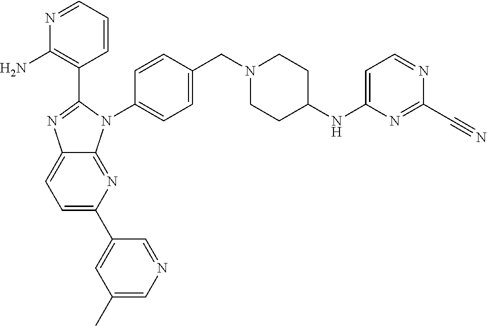 [SMILES: Cc1cncc(-c2ccc3nc(-c4cccnc4N)n(-c4ccc(CN5CCC(Nc6ccnc(C#N)n6)CC5)cc4)c3n2)c1] | 4-((1-(4-(2-(2-aminopyridin-3-yl)-5-(5- methylpyridin-3-yl)-3H-imidazo[4,5- b]pyridin-3-yl)benzyl)piperidin-4- yl)amino)pyrimidine-2-carbonitrile |
| 479 | 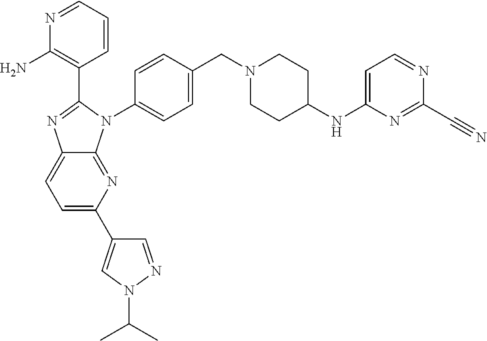 [SMILES: CC(C)n1cc(-c2ccc3nc(-c4cccnc4N)n(-c4ccc(CN5CCC(Nc6ccnc(C#N)n6)CC5)cc4)c3n2)cn1] | 4-((1-(4-(2-(2-aminopyridin-3-yl)-5-(1- isopropyl-1H-pyrazol-4-yl)-3H- imidazo[4,5-b]pyridin-3- yl)benzyl)piperidin-4-yl)amino)pyrimidine- 2-carbonitrile |
| 480 | 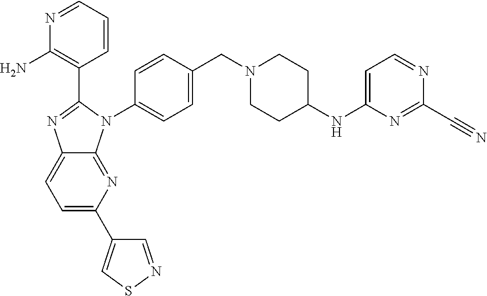 [SMILES: N#Cc1nccc(NC2CCN(Cc3ccc(-n4c(-c5cccnc5N)nc5ccc(-c6cnsc6)nc54)cc3)CC2)n1] | 4-((1-(4-(2-(2-aminopyridin-3-yl)-5- (isothiazol-4-yl)-3H-imidazo[4,5-b]pyridin- 3-yl)benzyl)piperidin-4- yl)amino)pyrimidine-2-carbonitrile |
| 481 | 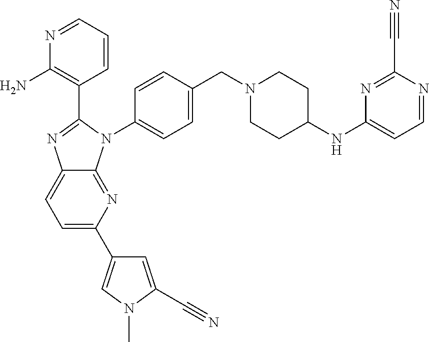 [SMILES: Cn1cc(-c2ccc3nc(-c4cccnc4N)n(-c4ccc(CN5CCC(Nc6ccnc(C#N)n6)CC5)cc4)c3n2)cc1C#N] | 4-((1-(4-(2-(2-aminopyridin-3-yl)-5-(5- cyano-1-methyl-1H-pyrrol-3-yl)-3H- imidazo[4,5-b]pyridin-3- yl)benzyl)piperidin-4-yl)amino)pyrimidine- 2-carbonitrile |
| 482 | 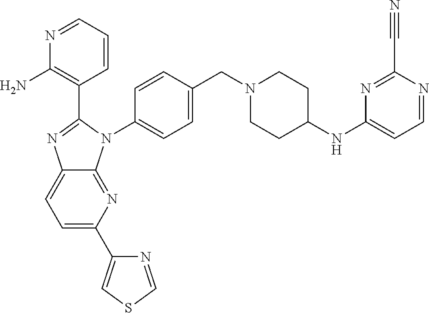 [SMILES: N#Cc1nccc(NC2CCN(Cc3ccc(-n4c(-c5cccnc5N)nc5ccc(-c6cscn6)nc54)cc3)CC2)n1] | 4-((1-(4-(2-(2-aminopyridin-3-yl)-5- (thiazol-4-yl)-3H-imidazo[4,5-b]pyridin-3- yl)benzyl)piperidin-4-yl)amino)pyrimidine- 2-carbonitrile |
| 483 | 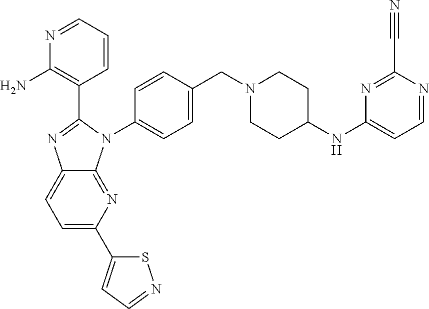 [SMILES: N#Cc1nccc(NC2CCN(Cc3ccc(-n4c(-c5cccnc5N)nc5ccc(-c6ccns6)nc54)cc3)CC2)n1] | 4-((1-(4-(2-(2-aminopyridin-3-yl)-5- (isothiazol-S-yl)-3H-imidazo[4,5-b]pyridin- 3-yl)benzyl)piperidin-4- yl)amino)pyrimidine-2-carbonitrile |
| 484 | 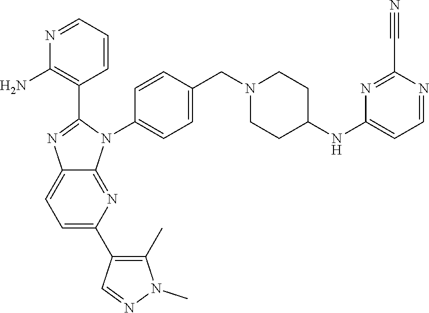 [SMILES: Cc1c(-c2ccc3nc(-c4cccnc4N)n(-c4ccc(CN5CCC(Nc6ccnc(C#N)n6)CC5)cc4)c3n2)cnn1C] | 4-((1-(4-(2-(2-aminopyridin-3-yl)-5-(1,5- dimethyl-1H-pyrazol-4-yl)-3H-imidazo[4,5- b]pyridin-3-yl)benzyl)piperidin-4- yl)amino)pyrimidine-2-carbonitrile |
| 485 | 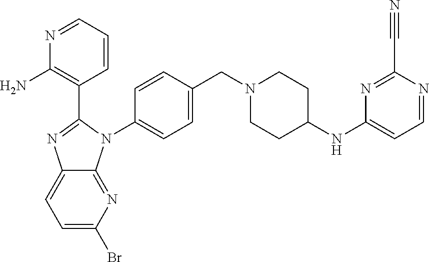 [SMILES: N#Cc1nccc(NC2CCN(Cc3ccc(-n4c(-c5cccnc5N)nc5ccc(Br)nc54)cc3)CC2)n1] | 4-((1-(4-(2-(2-aminopyridin-3-yl)-5-bromo- 3H-imidazo[4,5-b]pyridin-3- yl)benzyl)piperidin-4-yl)amino)pyrimidine- 2-carbonitrile |
| 486 | 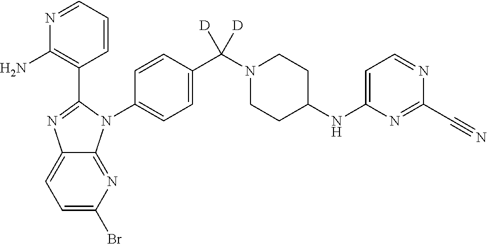 [SMILES: [2H]C([2H])(c1ccc(-n2c(-c3cccnc3N)nc3ccc(Br)nc32)cc1)N1CCC(Nc2ccnc(C#N)n2)CC1] | 4-((1-((4-(2-(2-aminopyridin-3-yl)-5- bromo-3H-imidazo[4,5-b]pyridin-3- yl)phenyl)methyl-d2)piperidin-4- yl)amino)pyrimidine-2-carbonitrile |
| 487 | 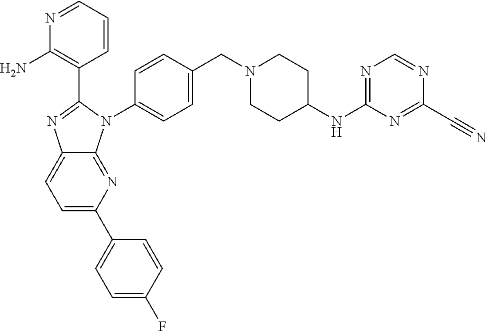 [SMILES: N#Cc1ncnc(NC2CCN(Cc3ccc(-n4c(-c5cccnc5N)nc5ccc(-c6ccc(F)cc6)nc54)cc3)CC2)n1] | 4-((1-(4-(2-(2-aminopyridin-3-yl)-5-(4- fluorophenyl)-3H-imidazo[4,5-b]pyridin-3- yl)benzyl)piperidin-4-yl)amino)-1,3,5- triazine-2-carbonitrile |
| 488 | 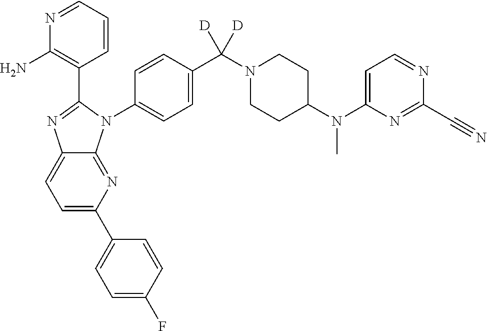 [SMILES: [2H]C([2H])(c1ccc(-n2c(-c3cccnc3N)nc3ccc(-c4ccc(F)cc4)nc32)cc1)N1CCC(N(C)c2ccnc(C#N)n2)CC1] | 4-((1-((4-(2-(2-aminopyridin-3-yl)-5-(4- fluorophenyl)-3H-imidazo[4,5-b]pyridin-3- yl)phenyl)methyl-d2)piperidin-4- yl)(methyl)amino)pyrimidine-2-carbonitrile |
| 489 | 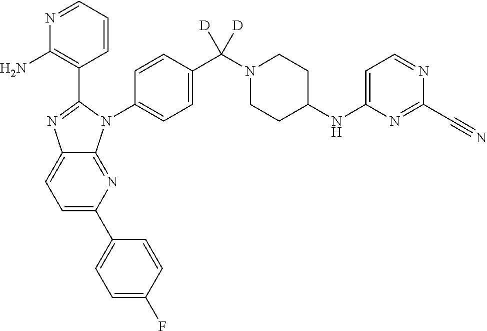 [SMILES: [2H]C([2H])(c1ccc(-n2c(-c3cccnc3N)nc3ccc(-c4ccc(F)cc4)nc32)cc1)N1CCC(Nc2ccnc(C#N)n2)CC1] | 4-((1-((4-(2-(2-aminopyridin-3-yl)-5-(4- fluorophenyl)-3H-imidazo[4,5-b]pyridin-3- yl)phenyl)methyl-d2)piperidin-4- yl)amino)pyrimidine-2-carbonitrile |
| 490 | 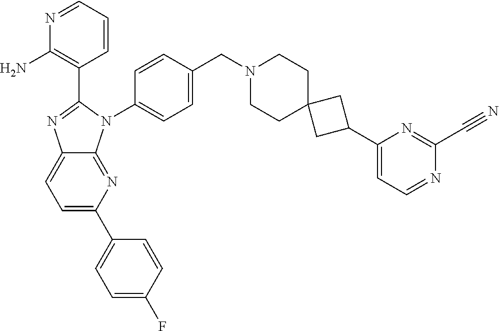 [SMILES: N#Cc1nccc(C2CC3(CCN(Cc4ccc(-n5c(-c6cccnc6N)nc6ccc(-c7ccc(F)cc7)nc65)cc4)CC3)C2)n1] | 4-(7-(4-(2-(2-aminopyridin-3-yl)-5-(4- fluorophenyl)-3H-imidazo[4,5-b]pyridin-3- yl)benzyl)-2,7-diazaspiro[3.5]nonan-2- yl)pyrimidine-2-carbonitrile |
| 491 | 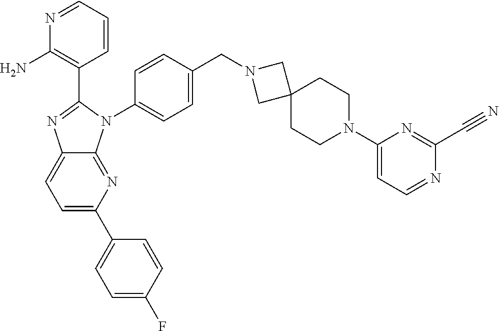 [SMILES: N#Cc1nccc(N2CCC3(CC2)CN(Cc2ccc(-n4c(-c5cccnc5N)nc5ccc(-c6ccc(F)cc6)nc54)cc2)C3)n1] | 4-(2-(4-(2-(2-aminopyridin-3-yl)-5-(4- fluorophenyl)-3H-imidazo[4,5-b]pyridin-3- yl)benzyl)-2,7-diazaspiro[3.5]nonan-7- yl)pyrimidine-2-carbonitrile |
| 492 | 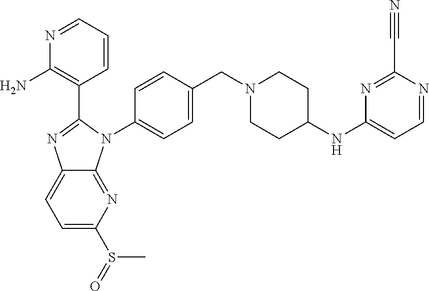 [SMILES: CS(=O)c1ccc2nc(-c3cccnc3N)n(-c3ccc(CN4CCC(Nc5ccnc(C#N)n5)CC4)cc3)c2n1] | 4-((1-(4-(2-(2-aminopyridin-3-yl)-5- (methylsulfinyl)-3H-imidazo[4,5-b]pyridin- 3-yl)benzyl)piperidin-4- yl)amino)pyrimidine-2-carbonitrile |
| 493 | 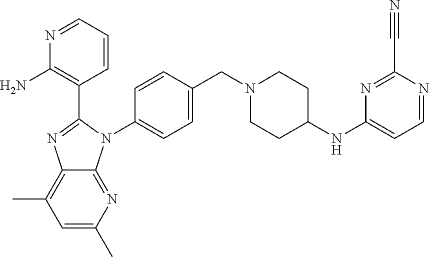 [SMILES: Cc1cc(C)c2nc(-c3cccnc3N)n(-c3ccc(CN4CCC(Nc5ccnc(C#N)n5)CC4)cc3)c2n1] | 4-((1-(4-(2-(2-aminopyridin-3-yl)-5,7- dimethyl-3H-imidazo[4,5-b]pyridin-3- yl)benzyl)piperidin-4-yl)amino)pyrimidine- 2-carbonitrile |
| 494 | 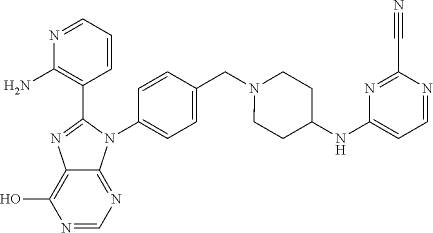 [SMILES: N#Cc1nccc(NC2CCN(Cc3ccc(-n4c(-c5cccnc5N)nc5c(O)ncnc54)cc3)CC2)n1] | 4-((1-(4-(8-(2-aminopyridin-3-yl)-6- hydroxy-9H-purin-9-yl)benzyl)piperidin-4- yl)amino)pyrimidine-2-carbonitrile |
| 495 | 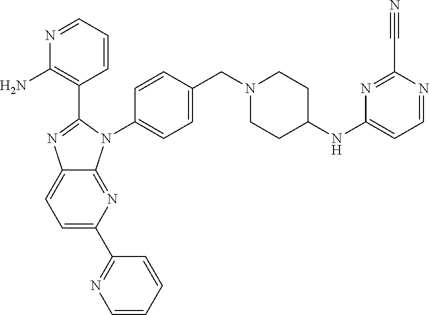 [SMILES: N#Cc1nccc(NC2CCN(Cc3ccc(-n4c(-c5cccnc5N)nc5ccc(-c6ccccn6)nc54)cc3)CC2)n1] | 4-((1-(4-(2-(2-aminopyridin-3-yl)-5- (pyridin-2-yl)- 3H-imidazo[4,5-b]pyridin-3- yl)benzyl)piperidin-4-yl)amino)pyrimidine- 2-carbonitrile |
| 496 | 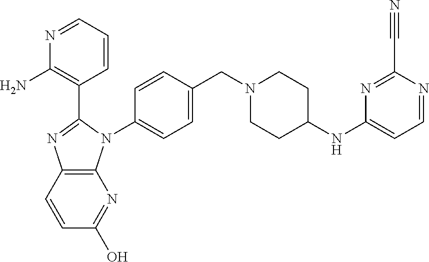 [SMILES: N#Cc1nccc(NC2CCN(Cc3ccc(-n4c(-c5cccnc5N)nc5ccc(O)nc54)cc3)CC2)n1] | 4-((1-(4-(2-(2-aminopyridin-3-yl)-5- hydroxy-3H-imidazo[4,5-b]pyridin-3- yl)benzyl)piperidin-4-yl)amino)pyrimidine- 2-carbonitrile |
| 497 | 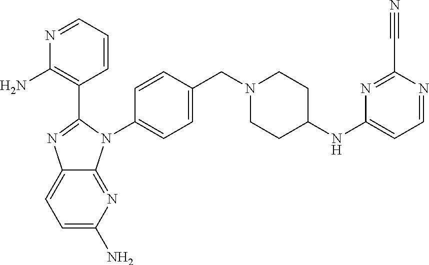 [SMILES: N#Cc1nccc(NC2CCN(Cc3ccc(-n4c(-c5cccnc5N)nc5ccc(N)nc54)cc3)CC2)n1] | 4-((1-(4-(5-amino-2-(2-aminopyridin-3-yl)- 3H-imidazo[4,5-b ]pyridin-3- yl)benzyl)piperidin-4-yl)amino)pyrimidine- 2-carbonitrile |
| 498 | 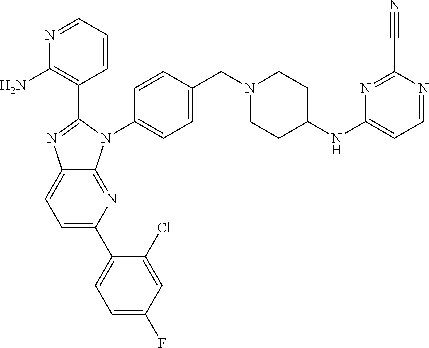 [SMILES: N#Cc1nccc(NC2CCN(Cc3ccc(-n4c(-c5cccnc5N)nc5ccc(-c6ccc(F)cc6Cl)nc54)cc3)CC2)n1] | 4-((1-(4-(2-(2-aminopyridin-3-yl)-5-(2- chloro-4-fluorophenyl)-3H-imidazo[4,5- b]pyridin-3-yl)benzyl)piperidin-4- yl)amino)pyrimidine-2-carbonitrile |
| 499 | 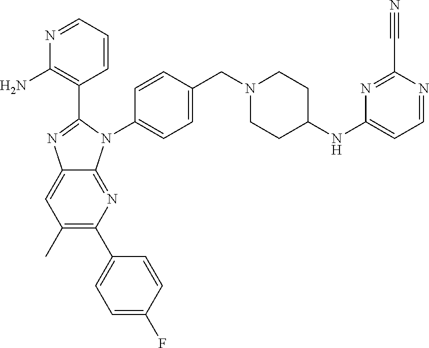 [SMILES: Cc1cc2nc(-c3cccnc3N)n(-c3ccc(CN4CCC(Nc5ccnc(C#N)n5)CC4)cc3)c2nc1-c1ccc(F)cc1] | 4-((1-(4-(2-(2-aminopyridin-3-yl)-5-(4- fluorophenyl)-6-methyl-3H-imidazo[4,5- b]pyridin-3-yl)benzyl)piperidin-4- yl)amino)pyrimidine-2-carbonitrile |
| 500 | 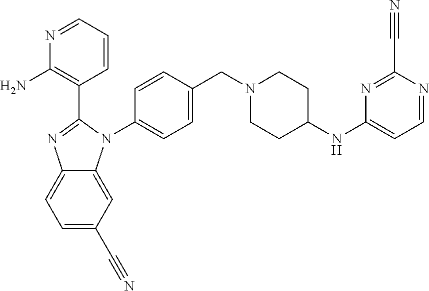 [SMILES: N#Cc1ccc2nc(-c3cccnc3N)n(-c3ccc(CN4CCC(Nc5ccnc(C#N)n5)CC4)cc3)c2c1] | 2-(2-aminopyridin-3-yl)-1-(4-((4-((2- cyanopyrimidin-4-yl)amino)piperidin-1- yl)methyl)phenyl)-1H-benzo[d]imidazole-6- carbonitrile |
| 501 | 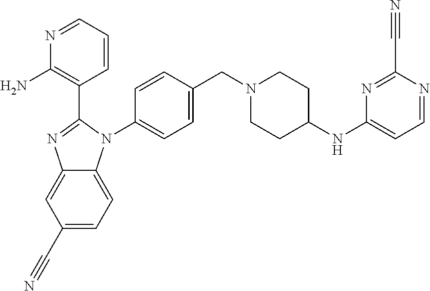 [SMILES: N#Cc1ccc2c(c1)nc(-c1cccnc1N)n2-c1ccc(CN2CCC(Nc3ccnc(C#N)n3)CC2)cc1] | 2-(2-aminopyridin-3-yl)-1-(4-((4-((2- cyanopyrimidin-4-yl)amino)piperidin-1- yl)methyl)phenyl)-1H-benzo[d]imidazole-5- carbonitrile |
| 502 | 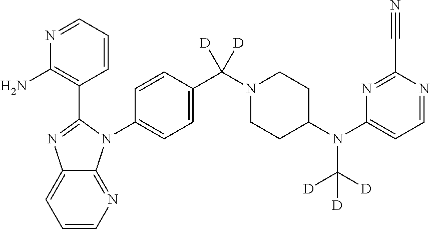 [SMILES: [2H]C([2H])([2H])N(c1ccnc(C#N)n1)C1CCN(C([2H])([2H])c2ccc(-n3c(-c4cccnc4N)nc4cccnc43)cc2)CC1] | 4-((1-((4-(2-(2-aminopyridin-3-yl)-3H- imidazo[4,5-b]pyridin-3-yl)phenyl)methyl- d2)piperidin-4-yl)(methyl- d3)amino)pyrimidine-2-carbonitrile |
| 503 | 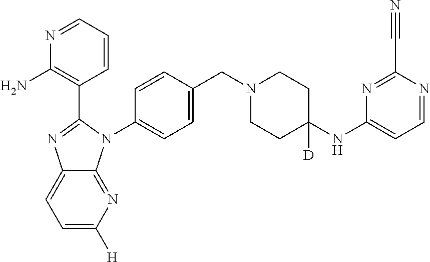 [SMILES: [2H]C1(Nc2ccnc(C#N)n2)CCN(Cc2ccc(-n3c(-c4cccnc4N)nc4cccnc43)cc2)CC1] | 4-((1-(4-(2-(2-aminopyridin-3-yl)-3H- imidazo[4,5-b]pyridin-3- yl)benzyl)piperidin-4-yl-4- d)amino)pyrimidine-2-carbonitrile |
| 504 | 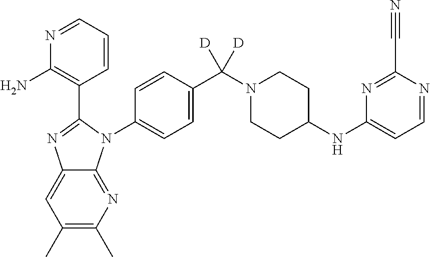 [SMILES: [2H]C([2H])(c1ccc(-n2c(-c3cccnc3N)nc3cc(C)c(C)nc32)cc1)N1CCC(Nc2ccnc(C#N)n2)CC1] | 4-((1-((4-(2-(2-aminopyridin-3-yl)-5,6- dimethyl-3H-imidazo[4,5-b]pyridin-3- yl)phenyl)methyl-d2)piperidin-4- yl)amino)pyrimidine-2-carbonitrile |
| 505 | 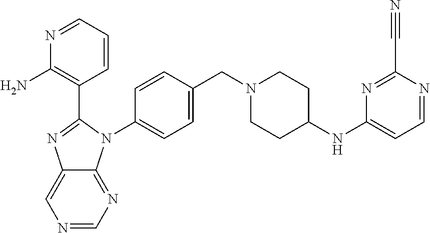 [SMILES: N#Cc1nccc(NC2CCN(Cc3ccc(-n4c(-c5cccnc5N)nc5cncnc54)cc3)CC2)n1] | 4-((1-(4-(8-(2-aminopyridin-3-yl)-9H-purin- 9-yl)benzyl)piperidin-4- yl)amino)pyrimidine-2-carbonitrile |
| 506 | 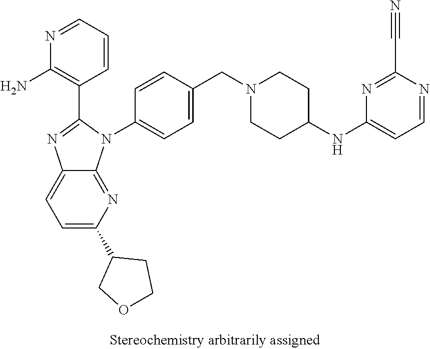 | (S)-4-((1-(4-(2-(2-aminopyridin- 3-yl)-5- (tetrahydrofuran-3-yl)-3H-imidazo[4,5- b]pyridin-3-yl)benzyl)piperidin-4- yl)amino)pyrimidine-2-carbonitrile |
| 507 | 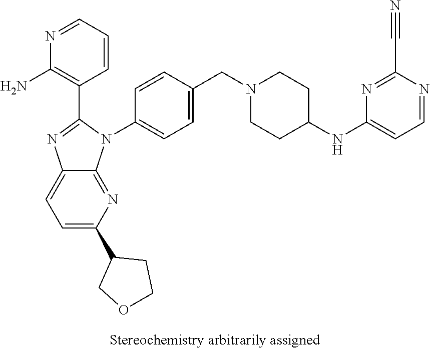 | (R)-4-((1-(4-(2-(2-aminopyridin-3-yl)-5- (tetrahydrofuran-3-yl)-3H-imidazo[4,5- b]pyridin-3-yl)benzyl)piperidin-4- yl)amino)pyrimidine-2-carbonitrile |
| 508 | 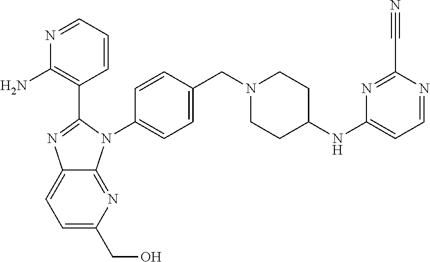 [SMILES: N#Cc1nccc(NC2CCN(Cc3ccc(-n4c(-c5cccnc5N)nc5ccc(CO)nc54)cc3)CC2)n1] | 4-((1-(4-(2-(2-aminopyridin-3-yl)-5- (hydroxymethyl)-3H-imidazo[4,5- b]pyridin-3-yl)benzyl)piperidin-4- yl)amino)pyrimidine-2-carbonitrile |
| 509 | 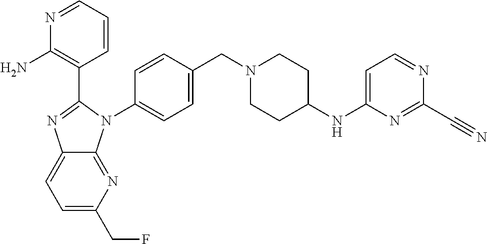 [SMILES: N#Cc1nccc(NC2CCN(Cc3ccc(-n4c(-c5cccnc5N)nc5ccc(CF)nc54)cc3)CC2)n1] | 4-((1-(4-(2-(2-aminopyridin-3-yl)-5- (fluoromethyl)-3H-imidazo[4,5-b]pyridin-3- yl)benzyl)piperidin-4-yl)amino)pyrimidine- 2-carbonitrile |
| 510 | 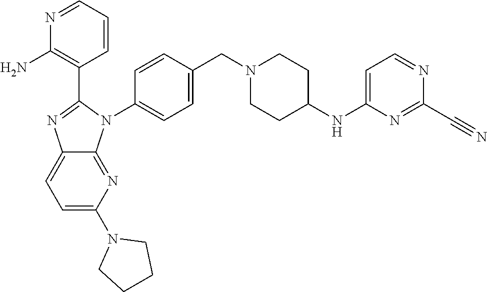 [SMILES: N#Cc1nccc(NC2CCN(Cc3ccc(-n4c(-c5cccnc5N)nc5ccc(N6CCCC6)nc54)cc3)CC2)n1] | 4-((1-(4-(2-(2-aminopyridin-3-yl)-5- (pyrrolidin-1-yl)-3H-imidazo[4,5-b]pyridin- 3-yl)benzyl)piperidin-4- yl)amino)pyrimidine-2-carbonitrile |
| 511 | 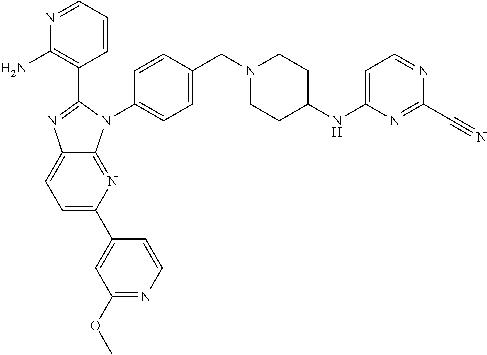 [SMILES: COc1cc(-c2ccc3nc(-c4cccnc4N)n(-c4ccc(CN5CCC(Nc6ccnc(C#N)n6)CC5)cc4)c3n2)ccn1] | 4-((1-(4-(2-(2-aminopyridin-3-yl)-5-(2- methoxypyridin-4-yl)-3H-imidazo[4,5- b]pyridin-3-yl)benzyl)piperidin-4- yl)amino)pyrimidine-2-carbonitrile |
| 512 | 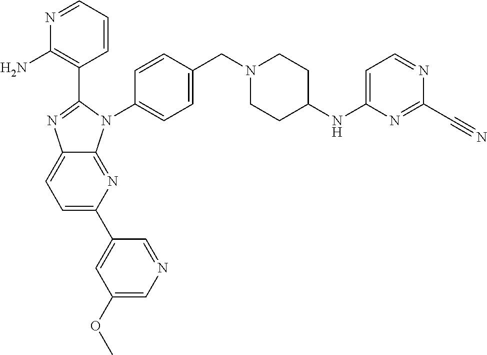 [SMILES: COc1cncc(-c2ccc3nc(-c4cccnc4N)n(-c4ccc(CN5CCC(Nc6ccnc(C#N)n6)CC5)cc4)c3n2)c1] | 4-((1-(4-(2-(2-aminopyridin-3-yl)-5-(5- methoxypyridin-3-yl)-3H-imidazo[4,5- b]pyridin-3-yl)benzyl)piperidin-4- yl)amino)pyrimidine-2-carbonitrile |
| 513 | 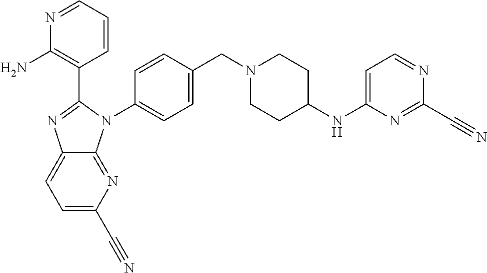 [SMILES: N#Cc1ccc2nc(-c3cccnc3N)n(-c3ccc(CN4CCC(Nc5ccnc(C#N)n5)CC4)cc3)c2n1] | 4-((1-(4-(2-(2-aminopyridin-3-yl)-5- ethynyl-3H-imidazo[4,5-b]pyridin-3- yl)benzyl)piperidin-4-yl)amino)pyrimidine- 2-carbonitrile |
| 514 | 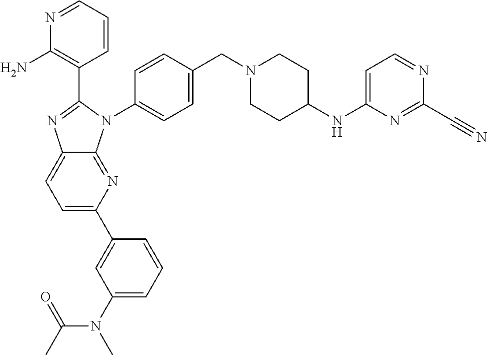 [SMILES: CC(=O)N(C)c1cccc(-c2ccc3nc(-c4cccnc4N)n(-c4ccc(CN5CCC(Nc6ccnc(C#N)n6)CC5)cc4)c3n2)c1] | N-(3-(2-(2-aminopyridin-3-yl)-3-(4-((4-((2- cyanopyrimidin-4-yl)amino)piperidin-1- yl)methyl)phenyl)-3H-imidazo[4,5- b]pyridin-5-yl)phenyl)-N-methylacetamide |
| 515 | 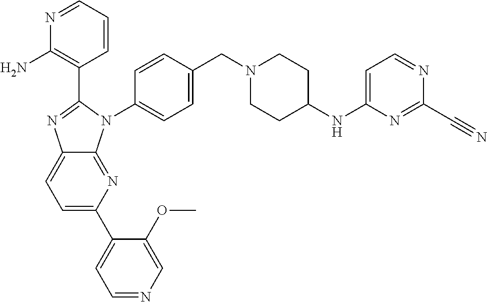 [SMILES: COc1cnccc1-c1ccc2nc(-c3cccnc3N)n(-c3ccc(CN4CCC(Nc5ccnc(C#N)n5)CC4)cc3)c2n1] | 4-((1-(4-(2-(2-aminopyridin-3-yl)-5-(3- methoxypyridin-4-yl)-3H-imidazo[4,5- b]pyridin-3-yl)benzyl)piperidin-4- yl)amino)pyrimidine-2-carbonitrile |
| 516 | 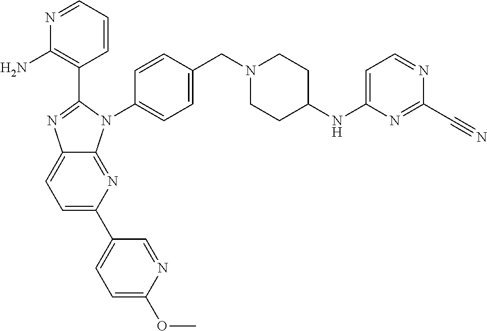 [SMILES: COc1ccc(-c2ccc3nc(-c4cccnc4N)n(-c4ccc(CN5CCC(Nc6ccnc(C#N)n6)CC5)cc4)c3n2)cn1] | 4-((1-(4-(2-(2-aminopyridin-3-yl)-5-(6- methoxypyridin-3-yl)-3H-imidazo[4,5- b]pyridin-3-yl)benzyl)piperidin-4- yl)amino)pyrimidine-2-carbonitrile |
| 517 | 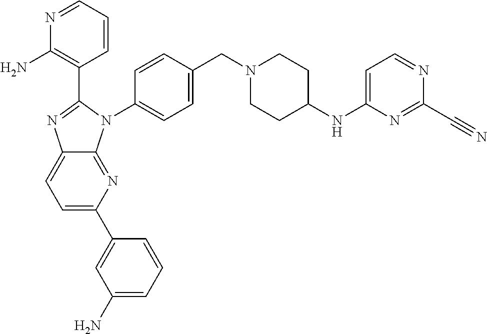 [SMILES: N#Cc1nccc(NC2CCN(Cc3ccc(-n4c(-c5cccnc5N)nc5ccc(-c6cccc(N)c6)nc54)cc3)CC2)n1] | 4-((1-(4-(5-(3-aminophenyl)-2-(2- aminopyridin-3-yl)-3H-imidazo[4,5- b]pyridin-3-yl)benzyl)piperidin-4- yl)amino)pyrimidine-2-carbonitrile |
| 518 | 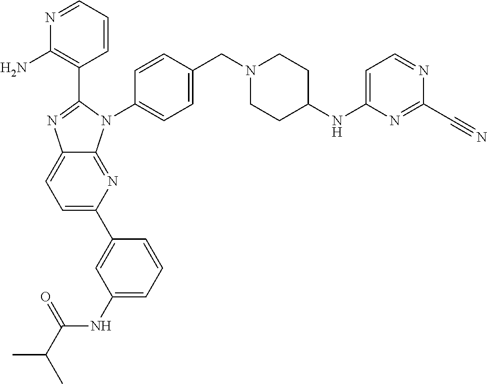 [SMILES: CC(C)C(=O)Nc1cccc(-c2ccc3nc(-c4cccnc4N)n(-c4ccc(CN5CCC(Nc6ccnc(C#N)n6)CC5)cc4)c3n2)c1] | N-(3-(2-(2-aminopyridin-3-yl)-3-(4-((4-((2- cyanopyrimidin-4-yl)amino)piperidin-1- yl)methyl)phenyl)-3H-imidazo[4,5- b]pyridin-5-yl)phenyl)isobutyramide |
| 519 | 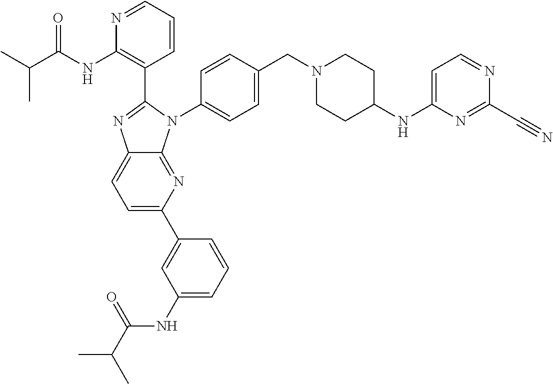 [SMILES: CC(C)C(=O)Nc1cccc(-c2ccc3nc(-c4cccnc4NC(=O)C(C)C)n(-c4ccc(CN5CCC(Nc6ccnc(C#N)n6)CC5)cc4)c3n2)c1] | N-(3-(3-(4-((4-((2-cyanopyrimidin-4- yl)amino)piperidin-1-yl)methyl)phenyl)-2- (2-isobutyramidopyridin-3-yl)-3H- imidazo[4,5-b]pyridin-5- yl)phenyl)isobutyramide |
| 520 | 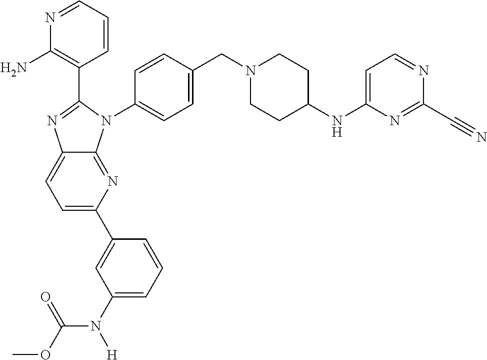 [SMILES: COC(=O)Nc1cccc(-c2ccc3nc(-c4cccnc4N)n(-c4ccc(CN5CCC(Nc6ccnc(C#N)n6)CC5)cc4)c3n2)c1] | methyl (3-(2-(2-aminopyridin-3-yl)-3-(4- ((4-((2-cyanopyrimidin-4- yl)amino)piperidin-1-yl)methyl)phenyl)- 3H-imidazo[4,5-b ]pyridin-5- yl)phenyl)carbamate |
| 521 | 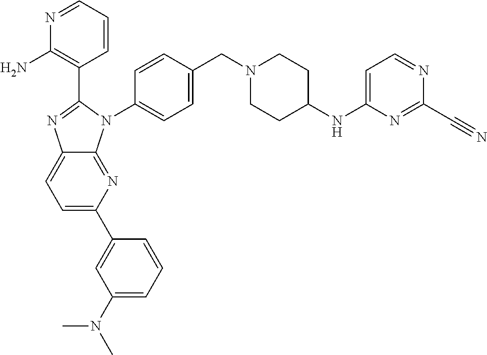 [SMILES: CN(C)c1cccc(-c2ccc3nc(-c4cccnc4N)n(-c4ccc(CN5CCC(Nc6ccnc(C#N)n6)CC5)cc4)c3n2)c1] | 4-((1-(4-(2-(2-aminopyridin-3-yl)-5-(3- (dimethylamino)phenyl)-3H-imidazo[4,5- b]pyridin-3-yl)benzyl)piperidin-4- yl)amino)pyrimidine-2-carbonitrile |
| 522 | 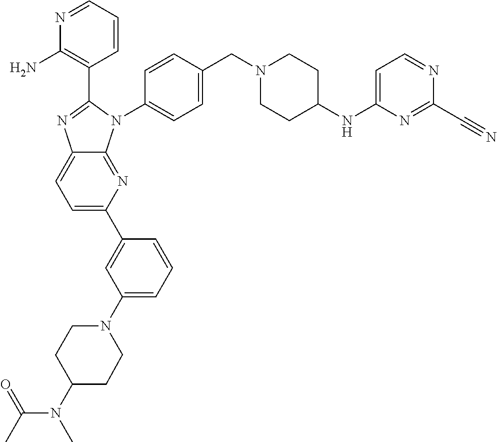 [SMILES: CC(=O)N(C)C1CCN(c2cccc(-c3ccc4nc(-c5cccnc5N)n(-c5ccc(CN6CCC(Nc7ccnc(C#N)n7)CC6)cc5)c4n3)c2)CC1] | N-(1-(3-(2-(2-aminopyridin-3-yl)-3-(4-((4- ((2-cyanopyrimidin-4-yl)amino)piperidin-1- yl)methyl)phenyl)-3H-imidazo[4,5- b]pyridin-5-yl)phenyl)piperidin-4-yl)-N- methylacetamide |
| 523 | 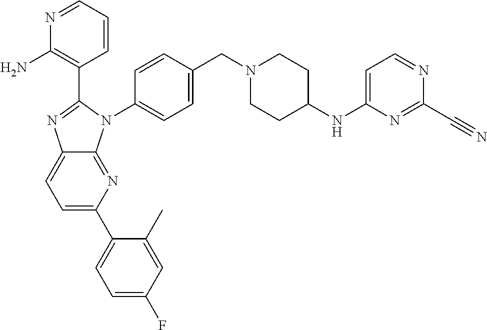 [SMILES: Cc1cc(F)ccc1-c1ccc2nc(-c3cccnc3N)n(-c3ccc(CN4CCC(Nc5ccnc(C#N)n5)CC4)cc3)c2n1] | 4-((1-(4-(2-(2-aminopyridin-3-yl)-5-(4- fluoro-2-methylphenyl)-3H-imidazo[4,5- b]pyridin-3-yl)benzyl)piperidin-4- yl)amino)pyrimidine-2-carbonitrile |
| 524 | 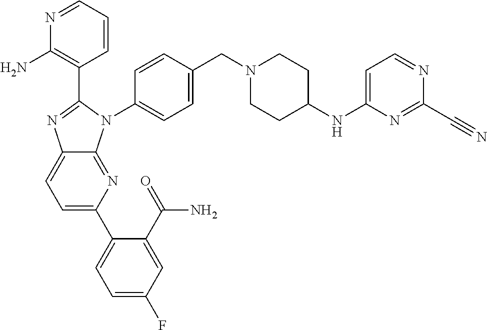 [SMILES: N#Cc1nccc(NC2CCN(Cc3ccc(-n4c(-c5cccnc5N)nc5ccc(-c6ccc(F)cc6C(N)=O)nc54)cc3)CC2)n1] | 2-(2-(2-aminopyridin-3-yl)-3-(4-((4-((2- cyanopyrimidin-4-yl)amino)piperidin-1- yl)methyl)phenyl)-3H-imidazo[4,5- b]pyridin-5-yl)-5-fluorobenzamide |
| 525 | 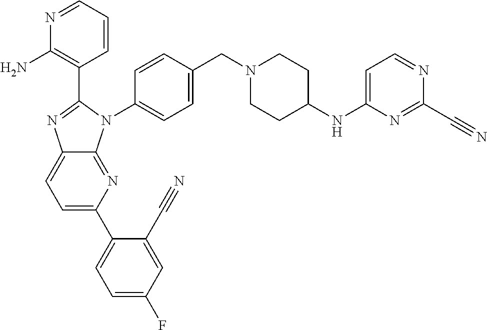 [SMILES: N#Cc1nccc(NC2CCN(Cc3ccc(-n4c(-c5cccnc5N)nc5ccc(-c6ccc(F)cc6C#N)nc54)cc3)CC2)n1] | 4-((1-(4-(2-(2-aminopyridin-3-yl)-5-(2- cyano-4-fluorophenyl)-3H-imidazo[4,5- b]pyridin-3-yl)benzyl)piperidin-4- yl)amino)pyrimidine-2-carbonitrile |
| 526 | 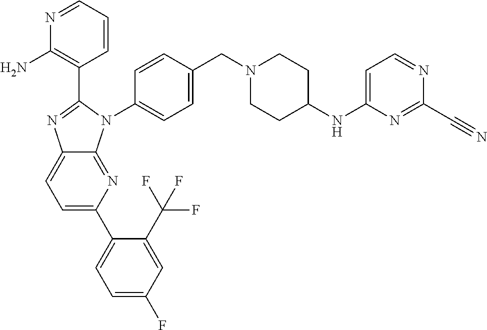 [SMILES: N#Cc1nccc(NC2CCN(Cc3ccc(-n4c(-c5cccnc5N)nc5ccc(-c6ccc(F)cc6C(F)(F)F)nc54)cc3)CC2)n1] | 4-((1-(4-(2-(2-aminopyridin-3-yl)-5-(4- fluoro-2-(trifluoromethyl)phenyl)-3H- imidazo[4,5-b]pyridin-3- yl)benzyl)piperidin-4-yl)amino)pyrimidine- 2-carbonitrile |
| 527 | 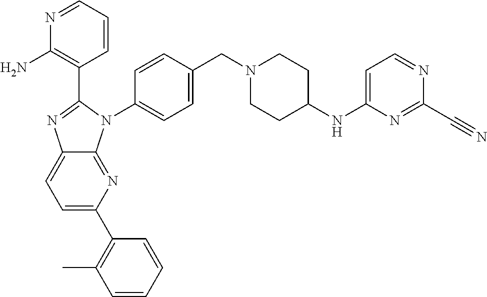 [SMILES: Cc1ccccc1-c1ccc2nc(-c3cccnc3N)n(-c3ccc(CN4CCC(Nc5ccnc(C#N)n5)CC4)cc3)c2n1] | 4-((1-(4-(2-(2-aminopyridin-3-yl)-5-(o- tolyl)-3H-imidazo[4,5-b]pyridin- 3- yl)benzyl)piperidin-4-yl)amino)pyrimidine- 2-carbonitrile |
| 528 | 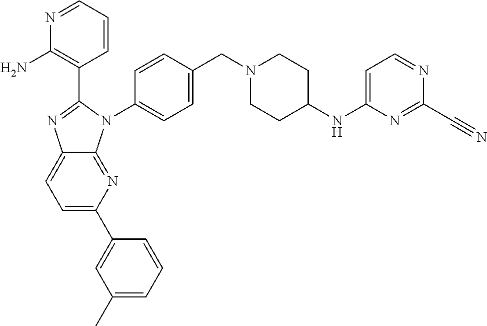 [SMILES: Cc1cccc(-c2ccc3nc(-c4cccnc4N)n(-c4ccc(CN5CCC(Nc6ccnc(C#N)n6)CC5)cc4)c3n2)c1] | 4-((1-(4-(2-(2-aminopyridin-3-yl)-5-(m- tolyl)-3H-imidazo[4,5-b]pyridin-3- yl)benzyl)piperidin-4-yl)amino)pyrimidine- 2-carbonitrile |
| 529 | 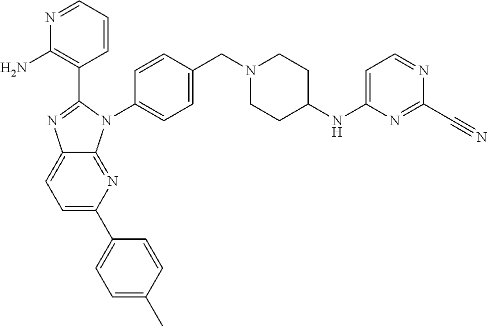 [SMILES: Cc1ccc(-c2ccc3nc(-c4cccnc4N)n(-c4ccc(CN5CCC(Nc6ccnc(C#N)n6)CC5)cc4)c3n2)cc1] | 4-((1-(4-(2-(2-aminopyridin-3-yl)-5-(p- tolyl)-3H-imidazo[4,5-b]pyridin-3- yl)benzyl)piperidin-4-yl)amino)pyrimidine- 2-carbonitrile |
| 530 | 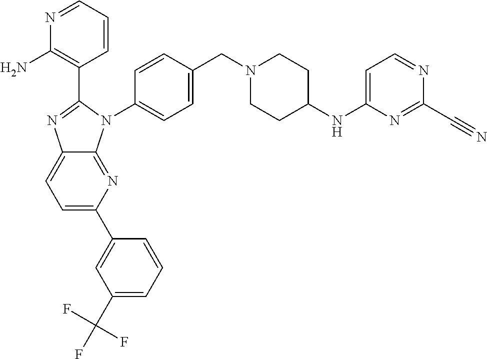 [SMILES: N#Cc1nccc(NC2CCN(Cc3ccc(-n4c(-c5cccnc5N)nc5ccc(-c6cccc(C(F)(F)F)c6)nc54)cc3)CC2)n1] | 4-((1-(4-(2-(2-aminopyridin-3-yl)-5-(3- (trifluoromethyl)phenyl)-3H-imidazo[4,5- b]pyridin-3-yl)benzyl)piperidin-4- yl)amino)pyrimidine-2-carbonitrile |
| 531 | 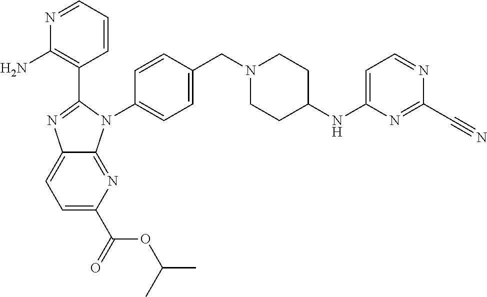 [SMILES: CC(C)OC(=O)c1ccc2nc(-c3cccnc3N)n(-c3ccc(CN4CCC(Nc5ccnc(C#N)n5)CC4)cc3)c2n1] | isopropyl 2-(2-aminopyridin-3-yl)-3-(4-((4- ((2-cyanopyrimidin-4-yl)amino)piperidin-1- yl)methyl)phenyl)-3H-imidazo[4,5- b]pyridine-5-carboxylate |
| 532 | 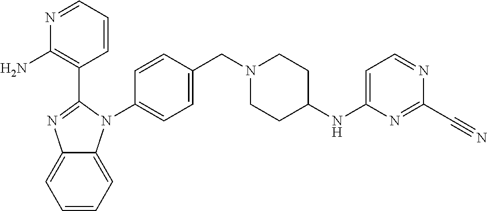 [SMILES: N#Cc1nccc(NC2CCN(Cc3ccc(-n4c(-c5cccnc5N)nc5ccccc54)cc3)CC2)n1] | 4-((1-(4-(2-(2-aminopyridin-3-yl)-1H- benzo[d]imidazol-1-yl)benzyl)piperidin-4- yl)amino)pyrimidine-2-carbonitrile +NL |
| 533 | 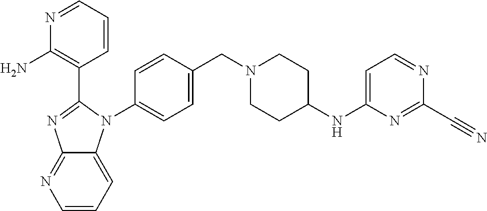 [SMILES: N#Cc1nccc(NC2CCN(Cc3ccc(-n4c(-c5cccnc5N)nc5ncccc54)cc3)CC2)n1] | 4-((1-(4-(2-(2-aminopyridin-3-yl)-1H- imidazo[4,5-b]pyridin-1- yl)benzyl)piperidin-4-yl)amino)pyrimidine- 2-carbonitrile |
| 534 | 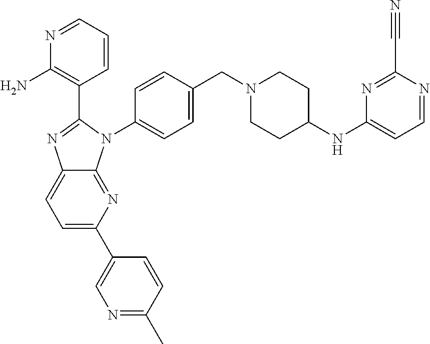 [SMILES: Cc1ccc(-c2ccc3nc(-c4cccnc4N)n(-c4ccc(CN5CCC(Nc6ccnc(C#N)n6)CC5)cc4)c3n2)cn1] | 4-((1- (4-(2-(2-aminopyridin-3-yl)-5-(6- methylpyridin-3-yl)-3H-imidazo[4,5- b]pyridin-3-yl)benzyl)piperidin-4- yl)amino)pyrimidine-2-carbonitrile |
| 535 | 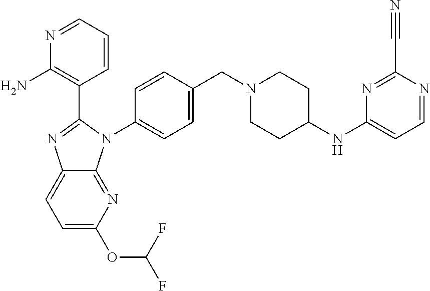 [SMILES: N#Cc1nccc(NC2CCN(Cc3ccc(-n4c(-c5cccnc5N)nc5ccc(OC(F)F)nc54)cc3)CC2)n1] | 4-((1-(4-(2-(2-aminopyridin-3-yl)-5- (difluoromethoxy)-3H-imidazo[4,5- b]pyridin-3-yl)benzyl)piperidin-4- yl)amino)pyrimidine-2-carbonitrile |
| 536 | 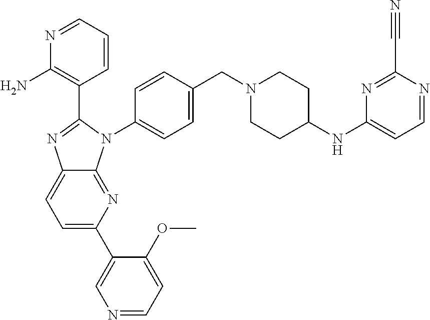 [SMILES: COc1ccncc1-c1ccc2nc(-c3cccnc3N)n(-c3ccc(CN4CCC(Nc5ccnc(C#N)n5)CC4)cc3)c2n1] | 4-((1-(4-(2-(2-aminopyridin-3-yl)-5-(4- methoxypyridin-3-yl)-3H-imidazo[4,5- b]pyridin-3-yl)benzyl)piperidin-4- yl)amino)pyrimidine-2-carbonitrile |
| 537 | 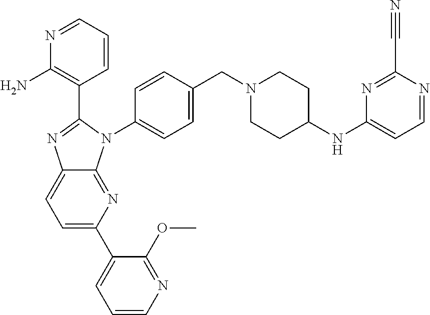 [SMILES: COc1ncccc1-c1ccc2nc(-c3cccnc3N)n(-c3ccc(CN4CCC(Nc5ccnc(C#N)n5)CC4)cc3)c2n1] | 4-((1-(4-(2-(2-aminopyridin-3-yl)-5-(2- methoxypyridin-3-yl)-3H-imidazo[4,5- b]pyridin-3-yl)benzyl)piperidin-4- yl)amino)pyrimidine-2-carbonitrile |
| 538 | 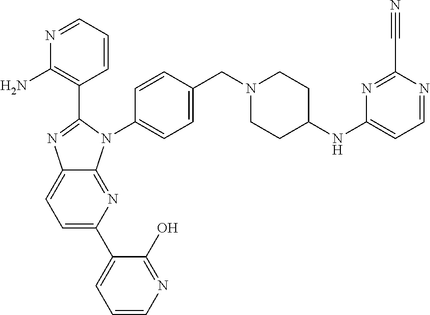 [SMILES: N#Cc1nccc(NC2CCN(Cc3ccc(-n4c(-c5cccnc5N)nc5ccc(-c6cccnc6O)nc54)cc3)CC2)n1] | 4-((1-(4-(2-(2-aminopyridin-3-yl)-5-(2- hydroxypyridin-3-yl)-3H-imidazo[4,5- b]pyridin-3-yl)benzyl)piperidin-4- yl)amino)pyrimidine-2-carbonitrile |
| 539 | 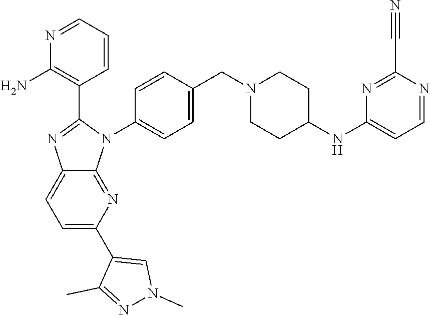 [SMILES: Cc1nn(C)cc1-c1ccc2nc(-c3cccnc3N)n(-c3ccc(CN4CCC(Nc5ccnc(C#N)n5)CC4)cc3)c2n1] | 4-((1-(4-(2-(2-aminopyridin-3-yl)-5-(1,3- dimethyl-1H-pyrazol-4-yl)-3H-imidazo[4,5- b]pyridin-3-yl)benzyl)piperidin-4- yl)amino)pyrimidine-2-carbonitrile |
| 540 | 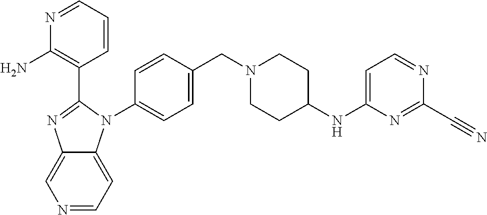 [SMILES: N#Cc1nccc(NC2CCN(Cc3ccc(-n4c(-c5cccnc5N)nc5cnccc54)cc3)CC2)n1] | 4-((1-(4-(2-(2-aminopyridin-3-yl)-1H- imidazo[4,5-c]pyridin-1- yl)benzyl)piperidin-4-yl)amino)pyrimidine- 2-carbonitrile |
| 541 | 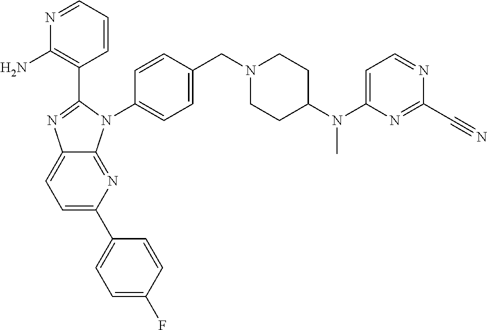 [SMILES: CN(c1ccnc(C#N)n1)C1CCN(Cc2ccc(-n3c(-c4cccnc4N)nc4ccc(-c5ccc(F)cc5)nc43)cc2)CC1] | 4-((1-(4-(2-(2-aminopyridin-3-yl)-5-(4- fluorophenyl)-3H-imidazo[4,5-b]pyridin-3- yl)benzyl)piperidin-4- yl)(methyl)amino)pyrimidine-2-carbonitrile |
| 542 | 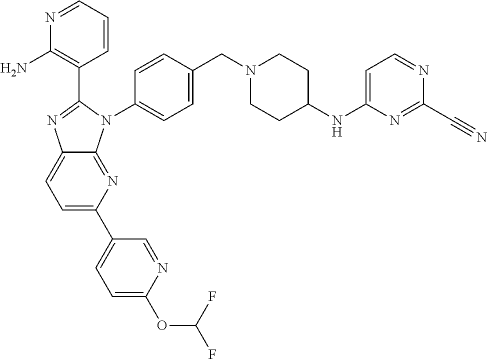 [SMILES: N#Cc1nccc(NC2CCN(Cc3ccc(-n4c(-c5cccnc5N)nc5ccc(-c6ccc(OC(F)F)nc6)nc54)cc3)CC2)n1] | 4-((1-(4-(2-(2-aminopyridin-3-yl)-5-(6- (difluoromethoxy)pyridin-3-yl)-3H- imidazo[4,5-b]pyridin-3- yl)benzyl)piperidin-4-yl)amino)pyrimidine- 2-carbonitrile |
| 543 | 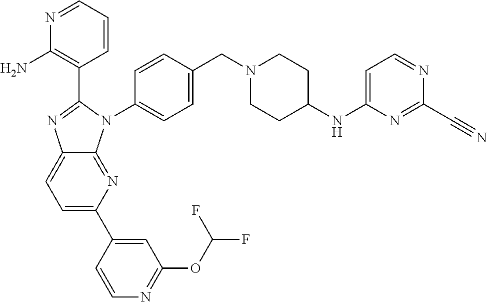 [SMILES: N#Cc1nccc(NC2CCN(Cc3ccc(-n4c(-c5cccnc5N)nc5ccc(-c6ccnc(OC(F)F)c6)nc54)cc3)CC2)n1] | 4-((1-(4-(2-(2-aminopyridin-3-yl)-5-(2- (difluoromethoxy)pyridin-4-yl)-3H- imidazo[4,5-b]pyridin-3- yl)benzyl)piperidin-4-yl)amino)pyrimidine- 2-carbonitrile |
| 544 | 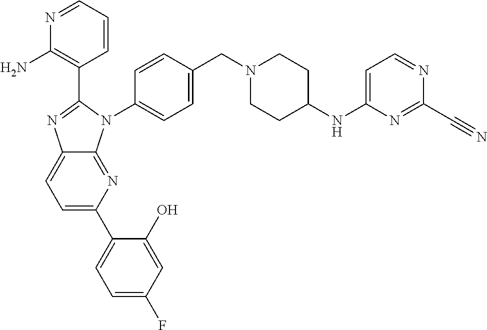 [SMILES: N#Cc1nccc(NC2CCN(Cc3ccc(-n4c(-c5cccnc5N)nc5ccc(-c6ccc(F)cc6O)nc54)cc3)CC2)n1] | 4-((1-(4-(2-(2-aminopyridin-3-yl)-5-(4- fluoro-2-hydroxyphenyl)-3H-imidazo[4,5- b]pyridin-3-yl)benzyl)piperidin-4- yl)amino)pyrimidine-2-carbonitrile |
| 545 | 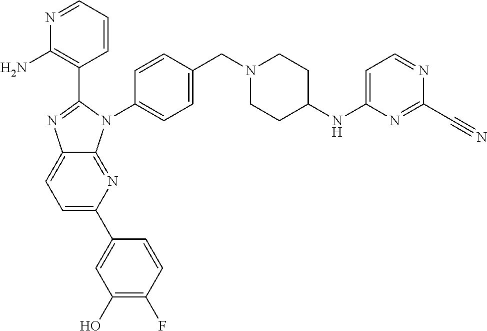 [SMILES: N#Cc1nccc(NC2CCN(Cc3ccc(-n4c(-c5cccnc5N)nc5ccc(-c6ccc(F)c(O)c6)nc54)cc3)CC2)n1] | 4-(1-(4-(2-(2-aminopyridin-3-yl)-5-(4- fluoro-3-hydroxyphenyl)-3H-imidazo[4,5- b]pyridin-3-yl)benzyl)piperidin-4- yl)amino)pyrimidine-2-carbonitrile |
| 546 | 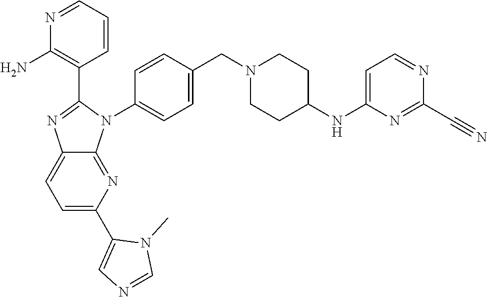 [SMILES: Cn1cncc1-c1ccc2nc(-c3cccnc3N)n(-c3ccc(CN4CCC(Nc5ccnc(C#N)n5)CC4)cc3)c2n1] | 4-((1-(4-(2-(2-aminopyridin-3-yl)-5-(1- methyl-1H-imidazol-5-yl)-3H-imidazo[4,5- b]pyridin-3-yl)benzyl)piperidin-4- yl)amino)pyrimidine-2-carbonitrile |
| 547 | 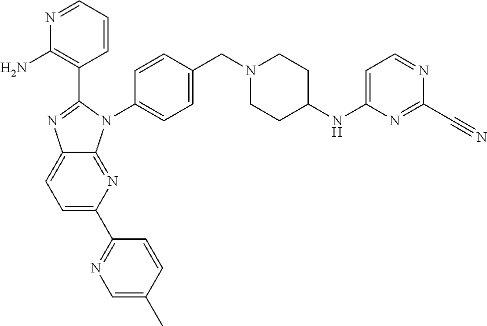 [SMILES: Cc1ccc(-c2ccc3nc(-c4cccnc4N)n(-c4ccc(CN5CCC(Nc6ccnc(C#N)n6)CC5)cc4)c3n2)nc1] | 4-((1-(4-(2-(2-aminopyridin-3-yl)-5-(5- methylpyridin-2-yl)-3H-imidazo[4,5- b]pyridin-3-yl)benzyl)piperidin-4- yl)amino)pyrimidine-2-carbonitrile |
| 548 | 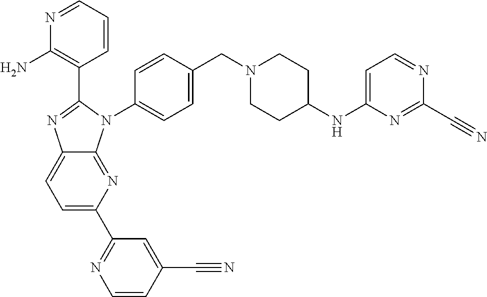 [SMILES: N#Cc1ccnc(-c2ccc3nc(-c4cccnc4N)n(-c4ccc(CN5CCC(Nc6ccnc(C#N)n6)CC5)cc4)c3n2)c1] | 4-((1-(4-(2-(2-aminopyridin-3-yl)-5-(4- cyanopyridin-2-yl)-3H-imidazo[4,5- b]pyridin-3-yl)benzyl)piperidin-4- yl)amino)pyrimidine-2-carbonitrile |
| 549 | 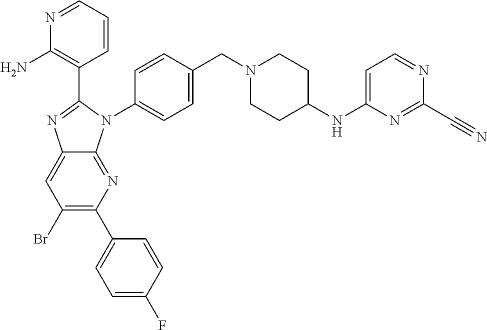 [SMILES: N#Cc1nccc(NC2CCN(Cc3ccc(-n4c(-c5cccnc5N)nc5cc(Br)c(-c6ccc(F)cc6)nc54)cc3)CC2)n1] | 4-((1-(4-(2-(2-aminopyridin-3-yl)-6-bromo- 5-(4-fluorophenyl)-3H-imidazo[4,5- b]pyridin-3-yl)benzyl)piperidin-4- yl)amino)pyrimidine-2-carbonitrile |
| 550 | 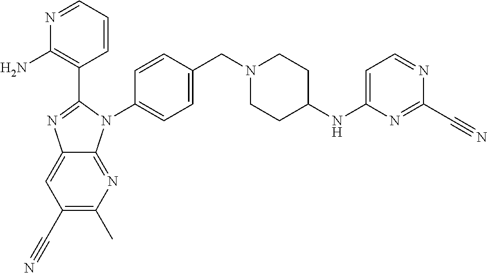 [SMILES: Cc1nc2c(cc1C#N)nc(-c1cccnc1N)n2-c1ccc(CN2CCC(Nc3ccnc(C#N)n3)CC2)cc1] | 2-(2-aminopyridin-3-yl)-3-(4-((4-((2- cyanopyrimidin-4-yl)amino)piperidin-1- yl)methyl)phenyl)-5-methyl-3H- imidazo[4,5-b]pyridine-6-carbonitrile |
| 551 | 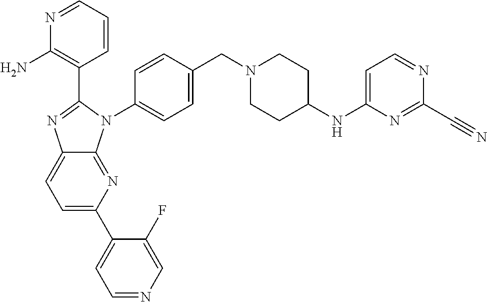 [SMILES: N#Cc1nccc(NC2CCN(Cc3ccc(-n4c(-c5cccnc5N)nc5ccc(-c6ccncc6F)nc54)cc3)CC2)n1] | 4-((1-(4-(2-(2-aminopyridin-3-yl)-5-(3- fluoropyridin-4-yl)-3H-imidazo[4,5- b]pyridin-3-yl)benzyl)piperidin-4- yl)amino)pyrimidine-2-carbonitrile |
| 552 | 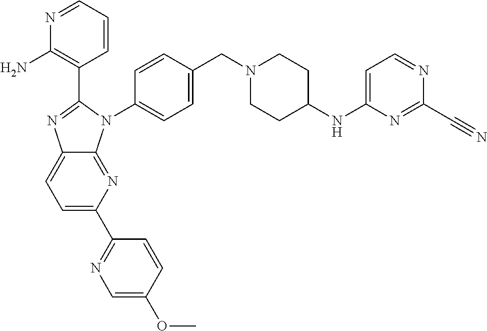 [SMILES: COc1ccc(-c2ccc3nc(-c4cccnc4N)n(-c4ccc(CN5CCC(Nc6ccnc(C#N)n6)CC5)cc4)c3n2)nc1] | 4-((1-(4-(2-(2-aminopyridin-3-yl)-5-(5- methoxypyridin-2-yl)-3H-imidazo[4,5- b]pyridin-3-yl)benzyl)piperidin-4- yl)amino)pyrimidine-2-carbonitrile |
| 553 | 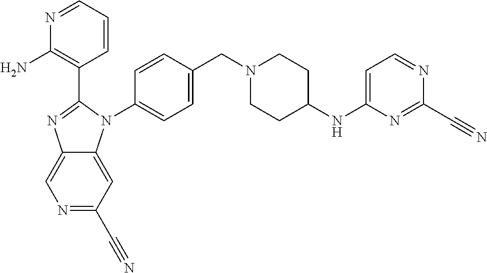 [SMILES: N#Cc1cc2c(cn1)nc(-c1cccnc1N)n2-c1ccc(CN2CCC(Nc3ccnc(C#N)n3)CC2)cc1] | 2-(2-aminopyridin-3-yl)-1-(4-((4-((2- cyanopyrimidin-4-yl)amino)piperidin-1- yl)methyl)phenyl)-1H-imidazo[4,5- c]pyridine-6-carbonitrile |
| 554 | 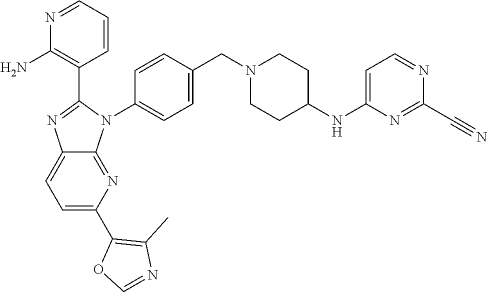 [SMILES: Cc1ncoc1-c1ccc2nc(-c3cccnc3N)n(-c3ccc(CN4CCC(Nc5ccnc(C#N)n5)CC4)cc3)c2n1] | 4-((1-(4-(2-(2-aminopyridin-3-yl)-5-(4- methyloxazol-5-yl)-3H-imidazo[4,5- b]pyridin-3-yl)benzyl)piperidin-4- yl)amino)pyrimidine-2-carbonitrile |
| 555 | 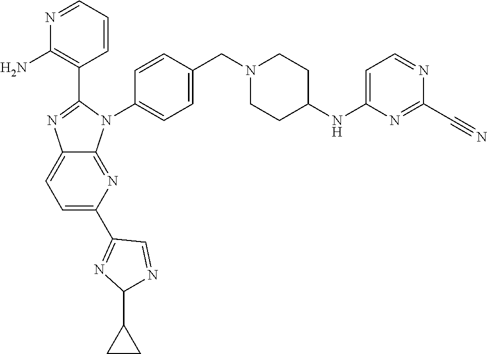 [SMILES: N#Cc1nccc(NC2CCN(Cc3ccc(-n4c(-c5cccnc5N)nc5ccc(C6=NC(C7CC7)N=C6)nc54)cc3)CC2)n1] | 4-((1-(4-(2-(2-aminopyridin-3-yl)-5-(1- cyclopropyl-1H-pyrazol-4-yl)-3H- imidazo[4,5-b]pyridin-3- yl)benzyl)piperidin-4-yl)amino)pyrimidine- 2-carbonitrile |
| 556 | 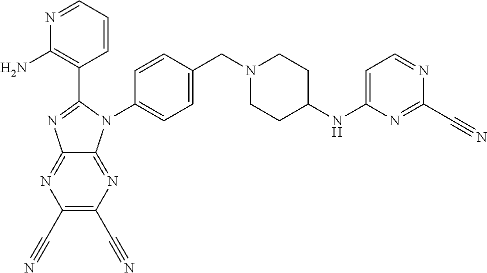 [SMILES: N#Cc1nccc(NC2CCN(Cc3ccc(-n4c(-c5cccnc5N)nc5nc(C#N)c(C#N)nc54)cc3)CC2)n1] | 2-(2-aminopyridin-3-yl)-1-(4-((4-((2- cyanopyrimidin-4-yl)amino)piperidin-1- yl)methyl)phenyl)-1H-imidazo[4,5- b]pyrazine-5,6-dicarbonitrile |
| 557 | 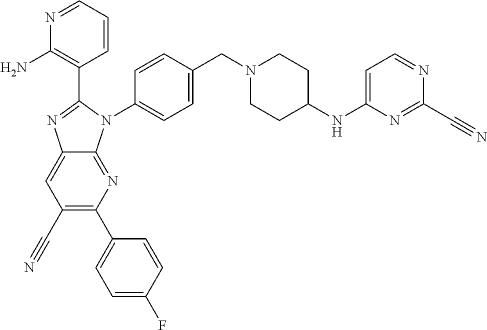 [SMILES: N#Cc1nccc(NC2CCN(Cc3ccc(-n4c(-c5cccnc5N)nc5cc(C#N)c(-c6ccc(F)cc6)nc54)cc3)CC2)n1] | 2-(2-aminopyridin-3-yl)-3-(4-((4-((2- cyanopyrimidin-4-yl)amino)piperidin-1- yl)methyl)phenyl)-5-(4-fluorophenyl)-3H- imidazo[4,5-b]pyridine-6-carbonitrile |
| 558 | 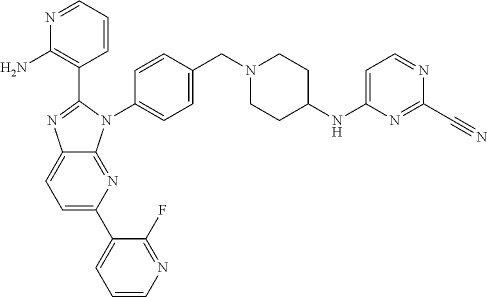 [SMILES: N#Cc1nccc(NC2CCN(Cc3ccc(-n4c(-c5cccnc5N)nc5ccc(-c6cccnc6F)nc54)cc3)CC2)n1] | 4-((1-(4-(2-(2-aminopyridin-3-yl)-5-(2- fluoropyridin-3-yl)-3H-imidazo[4,5- b]pyridin-3-yl)benzyl)piperidin-4- yl)amino)pyrimidine-2-carbonitrile |
| 559 | 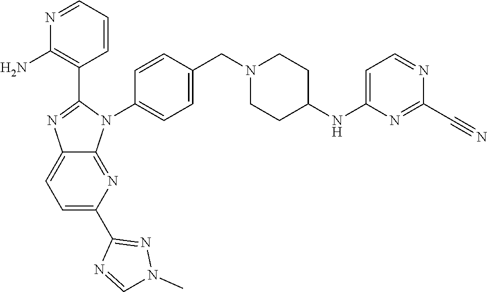 [SMILES: Cn1cnc(-c2ccc3nc(-c4cccnc4N)n(-c4ccc(CN5CCC(Nc6ccnc(C#N)n6)CC5)cc4)c3n2)n1] | 4-((1-(4-(2-(2-aminopyridin-3-yl)-5-(1- methyl-1H-1,2,4-triazol-3-yl)- 3H- imidazo[4,5-b]pyridin-3- yl)benzyl)piperidin-4-yl)amino)pyrimidine- 2-carbonitrile |
| 560 | 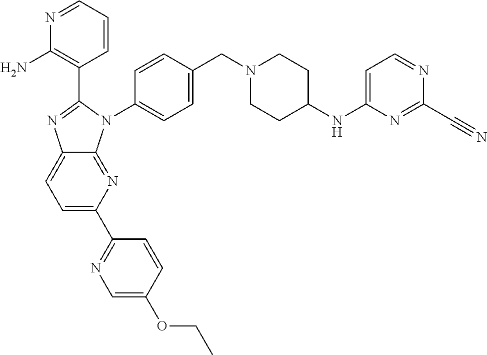 [SMILES: CCOc1ccc(-c2ccc3nc(-c4cccnc4N)n(-c4ccc(CN5CCC(Nc6ccnc(C#N)n6)CC5)cc4)c3n2)nc1] | 4-((1-(4-(2-(2-aminopyridin-3-yl)-5-(5- ethoxypyridin-2-yl)-3H-imidazo[4,5- b]pyridin-3-yl)benzyl)piperidin-4- yl)amino)pyrimidine-2-carbonitrile |
| 561 | 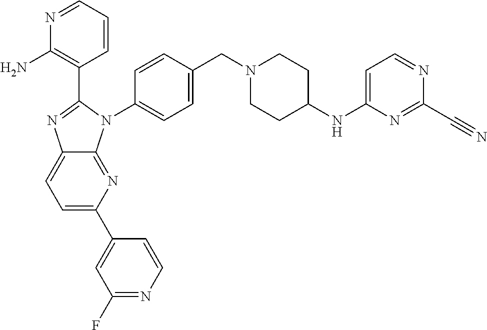 [SMILES: N#Cc1nccc(NC2CCN(Cc3ccc(-n4c(-c5cccnc5N)nc5ccc(-c6ccnc(F)c6)nc54)cc3)CC2)n1] | 4-((1-(4-(2-(2-aminopyridin-3-yl)-5-(2- fluoropyridin-4-yl)-3H-imidazo[4,5- b]pyridin-3-yl)benzyl)piperidin-4- yl)amino)pyrimidine-2-carbonitrile |
| 562 | 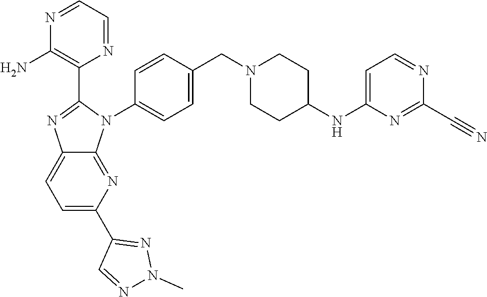 [SMILES: Cn1ncc(-c2ccc3nc(-c4nccnc4N)n(-c4ccc(CN5CCC(Nc6ccnc(C#N)n6)CC5)cc4)c3n2)n1] | 4-((1-(4-(2-(3-aminopyrazin-2-yl)-5-(2- methyl-2H-1,2,3-triazol-4-yl)-3H- imidazo[4,5-b]pyridin-3- yl)benzyl)piperidin-4-yl)amino)pyrimidine- 2-carbonitrile |
| 563 | 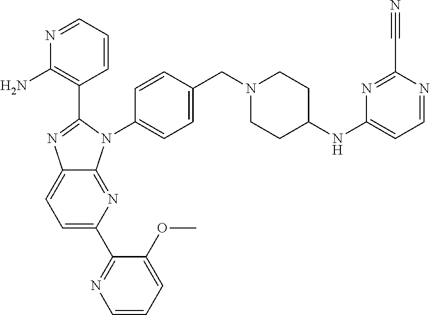 [SMILES: COc1cccnc1-c1ccc2nc(-c3cccnc3N)n(-c3ccc(CN4CCC(Nc5ccnc(C#N)n5)CC4)cc3)c2n1] | 4-((1-(4-(2-(2-aminopyridin-3-yl)-5-(3- methoxypyridin-2-yl)-3H-imidazo[4,5- b]pyridin-3-yl)benzyl)piperidin-4- yl)amino)pyrimidine-2-carbonitrile |
| 564 | 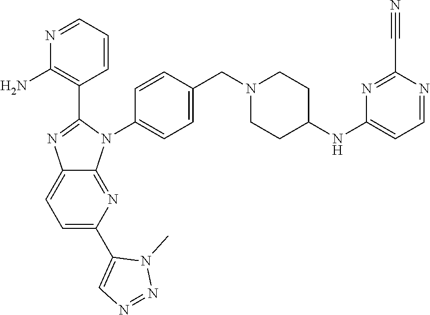 [SMILES: Cn1nncc1-c1ccc2nc(-c3cccnc3N)n(-c3ccc(CN4CCC(Nc5ccnc(C#N)n5)CC4)cc3)c2n1] | 4-((1-(4-(2-(2-aminopyridin-3-yl)-5-(1- methyl-1H-1,2,3-triazol-5-yl)-3H- imidazo[4,5-b]pyridin-3- yl)benzyl)piperidin-4-yl)amino)pyrimidine- 2-carbonitrile |
| 565 | 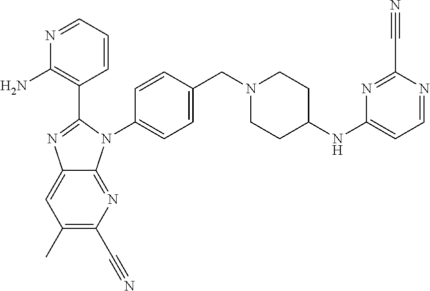 [SMILES: Cc1cc2nc(-c3cccnc3N)n(-c3ccc(CN4CCC(Nc5ccnc(C#N)n5)CC4)cc3)c2nc1C#N] | 2-(2-aminopyridin-3-yl)-3-(4-((4-((2- cyanopyrimidin-4-yl)amino)piperidin-1- yl)methyl)phenyl)-6-methyl-3H- imidazo[4,5-b]pyridine-5-carbonitrile |
| 566 | 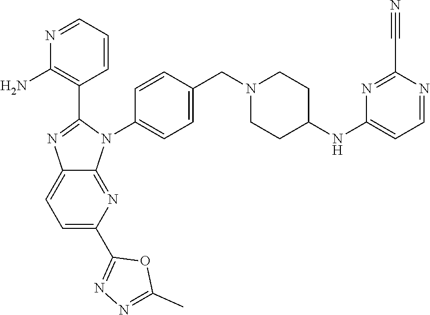 [SMILES: Cc1nnc(-c2ccc3nc(-c4cccnc4N)n(-c4ccc(CN5CCC(Nc6ccnc(C#N)n6)CC5)cc4)c3n2)o1] | 4-((1-(4-(2-(2-aminopyridin-3-yl)-5-(5- methyl-1,3,4-oxadiazol-2-yl)-3H- imidazo[4,5-b ]pyridin-3- yl)benzyl)piperidin-4-yl)amino)pyrimidine- 2-carbonitrile |
| 567 | 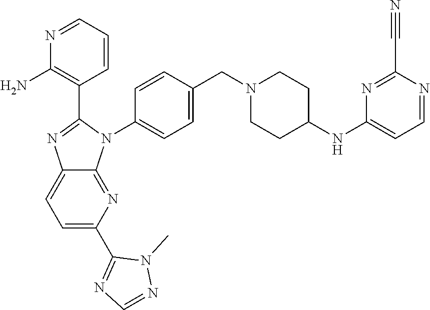 [SMILES: Cn1ncnc1-c1ccc2nc(-c3cccnc3N)n(-c3ccc(CN4CCC(Nc5ccnc(C#N)n5)CC4)cc3)c2n1] | 4-((1-(4-(2-(2-aminopyridin-3-yl)-5-(1- methyl-1H-1,2,4-triazol-5-yl)-3H- imidazo[4,5-b]pyridin-3- yl)benzyl)piperidin-4-yl)amino)pyrimidine- 2-carbonitrile |
| 568 | 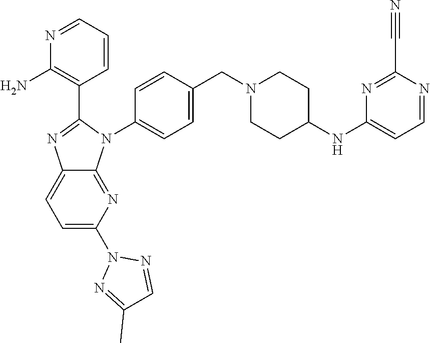 [SMILES: Cc1cnn(-c2ccc3nc(-c4cccnc4N)n(-c4ccc(CN5CCC(Nc6ccnc(C#N)n6)CC5)cc4)c3n2)n1] | 4-((1- (4-(2-(2-aminopyridin-3-yl)-5-(4- methyl-2H-1,2,3-triazol-2-yl)-3H- imidazo[4,5-b]pyridin-3- yl)benzyl)piperidin-4-yl)amino)pyrimidine- 2-carbonitrile |
| 569 | 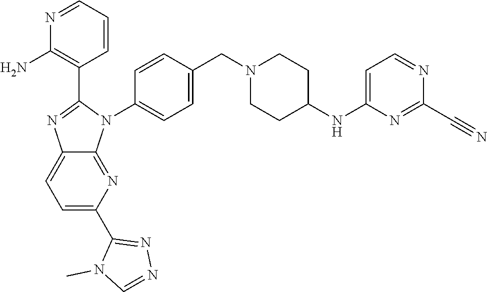 [SMILES: Cn1cnnc1-c1ccc2nc(-c3cccnc3N)n(-c3ccc(CN4CCC(Nc5ccnc(C#N)n5)CC4)cc3)c2n1] | 4-((1-(4-(2-(2-aminopyridin-3-yl)-5-(4- methyl-4H-1,2,4-triazol-3-yl)-3H- imidazo[4,5-b]pyridin-3- yl)benzyl)piperidin-4-yl)amino)pyrimidine- 2-carbonitrile |
| 570 | 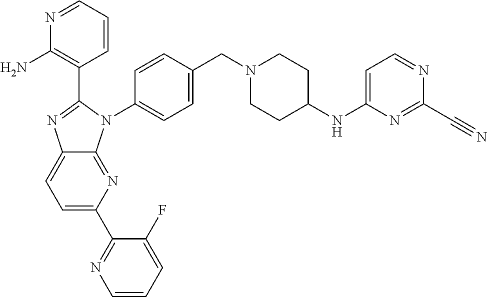 [SMILES: N#Cc1nccc(NC2CCN(Cc3ccc(-n4c(-c5cccnc5N)nc5ccc(-c6ncccc6F)nc54)cc3)CC2)n1] | 4-((1-(4-(2-(2-aminopyridin-3-yl)-5-(3- fluoropyridin-2-yl)-3H-imidazo[4,5- b]pyridin-3-yl)benzyl)piperidin-4- yl)amino)pyrimidine-2-carbonitrile |
| 571 | 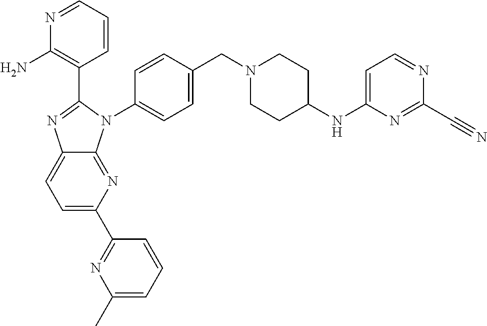 [SMILES: Cc1cccc(-c2ccc3nc(-c4cccnc4N)n(-c4ccc(CN5CCC(Nc6ccnc(C#N)n6)CC5)cc4)c3n2)n1] | 4-((1-(4-(2-(2-aminopyridin-3-yl)-5-(6- methylpyridin-2-yl)-3H-imidazo[4,5- b]pyridin-3-yl)benzyl)piperidin-4- yl)amino)pyrimidine-2-carbonitrile |
| 572 | 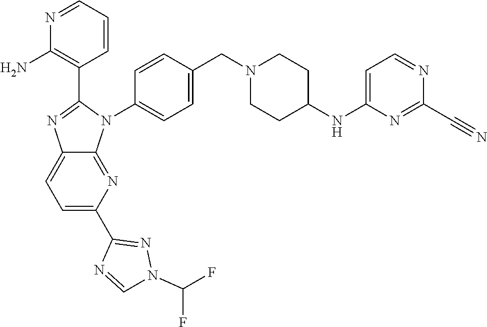 [SMILES: N#Cc1nccc(NC2CCN(Cc3ccc(-n4c(-c5cccnc5N)nc5ccc(-c6ncn(C(F)F)n6)nc54)cc3)CC2)n1] | 4-((1-(4-(2-(2-aminopyridin-3-yl)-5-(1- (difluoromethyl)-1H-1,2,4-triazol-3-yl)-3H- imidazo[4,5-b]pyridin-3- yl)benzyl)piperidin-4-yl)amino)pyrimidine- 2-carbonitrile |
| 573 | 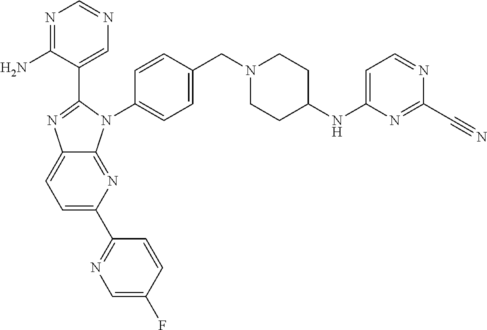 [SMILES: N#Cc1nccc(NC2CCN(Cc3ccc(-n4c(-c5cncnc5N)nc5ccc(-c6ccc(F)cn6)nc54)cc3)CC2)n1] | 4-((1-(4-(2-(4-aminopyrimidin-5-yl)-5-(5- fluoropyridin-2-yl)-3H-imidazo[4,5- b]pyridin-3-yl)benzyl)piperidin-4- yl)amino)pyrimidine-2-carbonitrile |
| 574 | 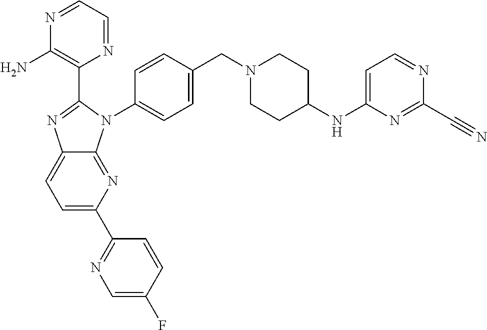 [SMILES: N#Cc1nccc(NC2CCN(Cc3ccc(-n4c(-c5nccnc5N)nc5ccc(-c6ccc(F)cn6)nc54)cc3)CC2)n1] | 4-((1-(4-(2-(3-aminopyrazin-2-yl)-5-(5- fluoropyridin-2-yl)-3H-imidazo[4,5- b]pyridin-3-yl)benzyl)piperidin-4- yl)amino)pyrimidine-2-carbonitrile |
| 575 | 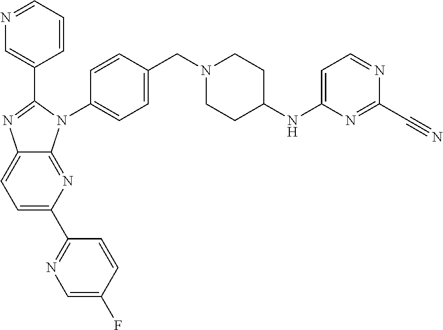 [SMILES: N#Cc1nccc(NC2CCN(Cc3ccc(-n4c(-c5cccnc5)nc5ccc(-c6ccc(F)cn6)nc54)cc3)CC2)n1] | 4-((1-(4-(5-(5-fluoropyridin-2-yl)-2- (pyridin-3-yl)-3H-imidazo[4,5-b]pyridin-3- yl)benzyl)piperidin-4-yl)amino)pyrimidine- 2-carbonitrile |
| 576 | 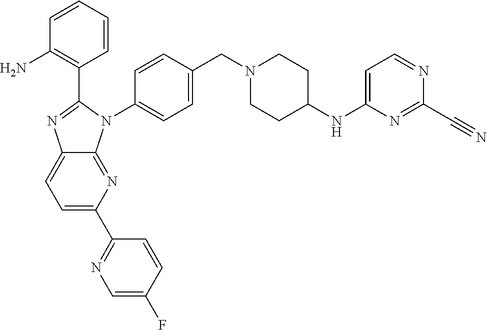 [SMILES: N#Cc1nccc(NC2CCN(Cc3ccc(-n4c(-c5ccccc5N)nc5ccc(-c6ccc(F)cn6)nc54)cc3)CC2)n1] | 4-((1-(4-(2-(2-aminophenyl)-5-(5- fluoropyridin-2-yl)-3H-imidazo[4,5- b]pyridin-3-yl)benzyl)piperidin-4- yl)amino)pyrimidine-2-carbonitrile |
| 577 | 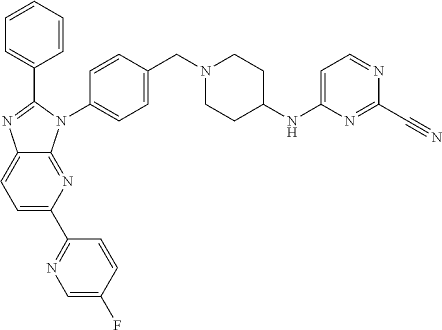 [SMILES: N#Cc1nccc(NC2CCN(Cc3ccc(-n4c(-c5ccccc5)nc5ccc(-c6ccc(F)cn6)nc54)cc3)CC2)n1] | 4-((1-(4-(5-(5-fluoropyridin-2-yl)-2-phenyl- 3H-imidazo[4,5-b]pyridin-3- yl)benzyl)piperidin-4-yl)amino)pyrimidine- 2-carbonitrile |
| 578 | 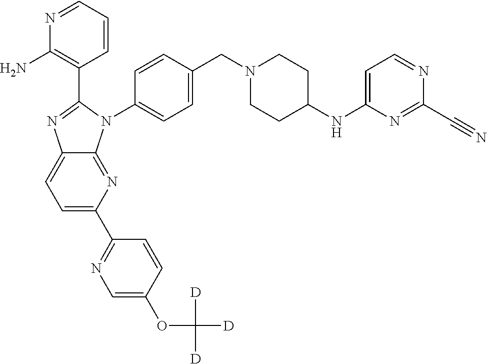 [SMILES: [2H]C([2H])([2H])Oc1ccc(-c2ccc3nc(-c4cccnc4N)n(-c4ccc(CN5CCC(Nc6ccnc(C#N)n6)CC5)cc4)c3n2)nc1] | 4-((1-(4-(2-(2-aminopyridin-3-yl)-5-(5- (methoxy-d3)pyridin-2-yl)-3H-imidazo[4,5- b]pyridin-3-yl)benzyl)piperidin-4- yl)amino)pyrimidine-2-carbonitrile |
| 579 | 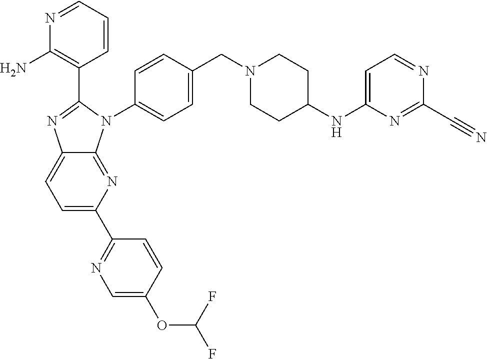 [SMILES: N#Cc1nccc(NC2CCN(Cc3ccc(-n4c(-c5cccnc5N)nc5ccc(-c6ccc(OC(F)F)cn6)nc54)cc3)CC2)n1] | 4-((1-(4-(2-(2-aminopyridin-3-yl)-5-(5- (difluoromethoxy)pyridin-2-yl)-3H- imidazo[4,5-b]pyridin-3- yl)benzyl)piperidin-4-yl)amino)pyrimidine- 2-carbonitrile |
| 580 | 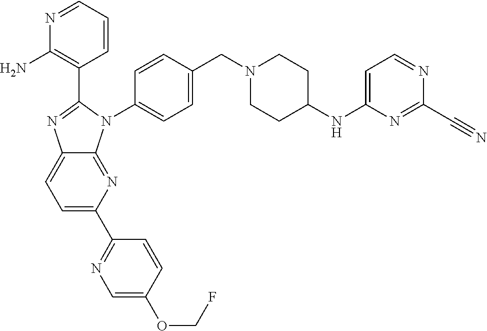 [SMILES: N#Cc1nccc(NC2CCN(Cc3ccc(-n4c(-c5cccnc5N)nc5ccc(-c6ccc(OCF)cn6)nc54)cc3)CC2)n1] | 4-((1-(4-(2-(2-aminopyridin-3-yl)-5-(5- (fluoromethoxy)pyridin-2-yl)-3H- imidazo[4,5-b]pyridin-3- yl)benzyl)piperidin-4-yl)amino)pyrimidine- 2-carbonitrile |
| 581 | 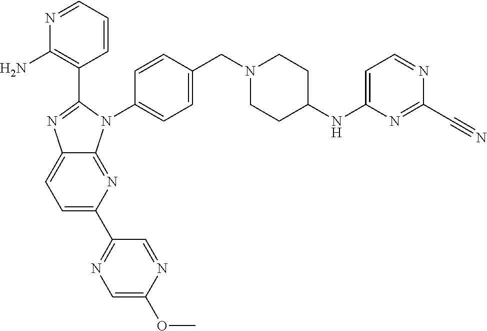 [SMILES: COc1cnc(-c2ccc3nc(-c4cccnc4N)n(-c4ccc(CN5CCC(Nc6ccnc(C#N)n6)CC5)cc4)c3n2)cn1] | 4-((1-(4-(2-(2-aminopyridin-3-yl)-5-(5- methoxypyrimidin-2-yl)-3H-imidazo[4,5- b]pyridin-3-yl)benzyl)piperidin-4- yl)amino)pyrimidine-2-carbonitrile |
| 582 | 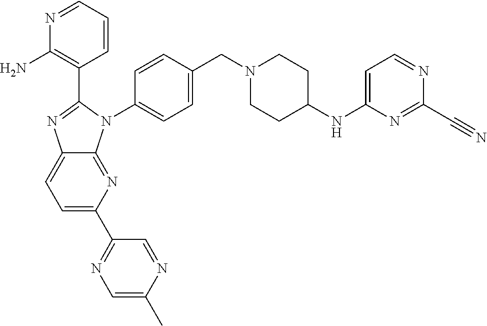 [SMILES: Cc1cnc(-c2ccc3nc(-c4cccnc4N)n(-c4ccc(CN5CCC(Nc6ccnc(C#N)n6)CC5)cc4)c3n2)cn1] | 4-((1-(4-(2-(2-aminopyridin-3-yl)-5-(5- methylpyrazin-2-yl)-3H-imidazo[4,5- b]pyridin-3-yl)benzyl)piperidin-4- yl)amino)pyrimidine-2-carbonitrile |
| 583 | 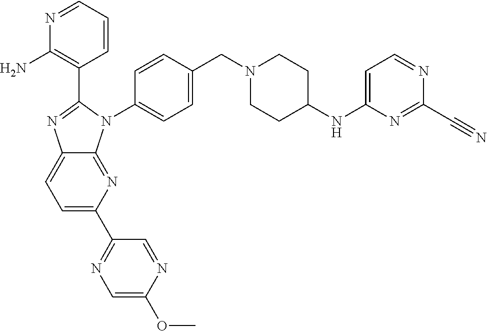 [SMILES: COc1cnc(-c2ccc3nc(-c4cccnc4N)n(-c4ccc(CN5CCC(Nc6ccnc(C#N)n6)CC5)cc4)c3n2)cn1] | 4-((1-(4-(2-(2-aminopyridin-3-yl)-5-(5- methoxypyrazin-2-yl)-3H-imidazo[4,5- b]pyridin-3-yl)benzyl)piperidin-4- yl)amino)pyrimidine-2-carbonitrile |
| 584 | 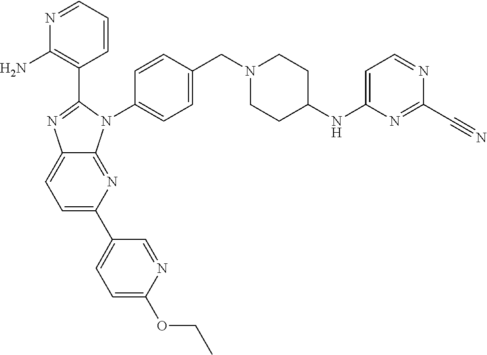 [SMILES: CCOc1ccc(-c2ccc3nc(-c4cccnc4N)n(-c4ccc(CN5CCC(Nc6ccnc(C#N)n6)CC5)cc4)c3n2)cn1] | 4-((1-(4-(2-(2-aminopyridin-3-yl)-5-(6- ethoxypyridin-3-yl)-3H-imidazo[4,5- b]pyridin-3-yl)benzyl)piperidin-4- yl)amino)pyrimidine-2-carbonitrile |
| 585 | 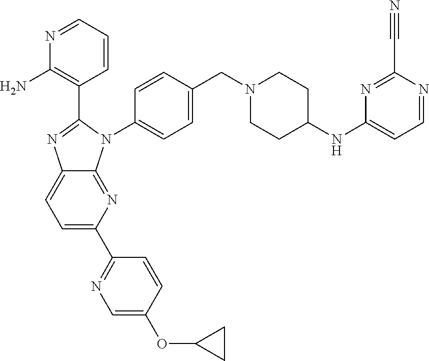 [SMILES: N#Cc1nccc(NC2CCN(Cc3ccc(-n4c(-c5cccnc5N)nc5ccc(-c6ccc(OC7CC7)cn6)nc54)cc3)CC2)n1] | 4-((1-(4-(2-(2-aminopyridin-3-yl)-5-(5- cyclopropoxypyridin-2-yl)-3H-imidazo[4,5- b]pyridin-3-yl)benzyl)piperidin-4- yl)amino)pyrimidine-2-carbonitrile |
| 586 | 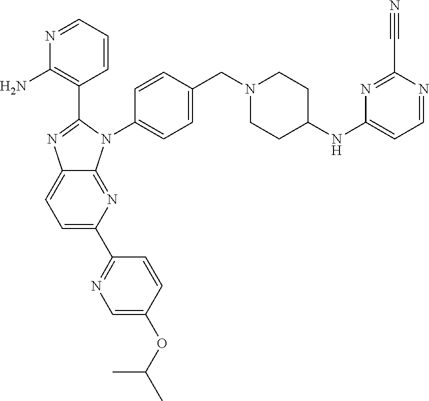 [SMILES: CC(C)Oc1ccc(-c2ccc3nc(-c4cccnc4N)n(-c4ccc(CN5CCC(Nc6ccnc(C#N)n6)CC5)cc4)c3n2)nc1] | 4-((1-(4-(2-(2-aminopyridin-3-yl)-5-(5- isopropoxypyridin-2-yl)-3H-imidazo[4,5- b]pyridin-3-yl)benzyl)piperidin-4- yl)amino)pyrimidine-2-carbonitrile |
| 587 | 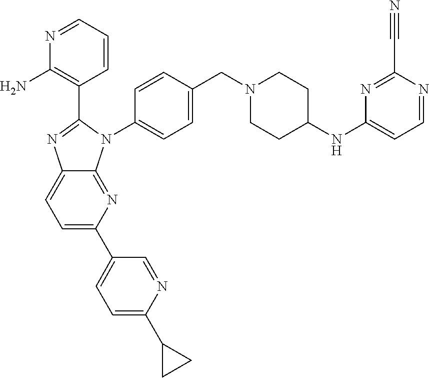 [SMILES: N#Cc1nccc(NC2CCN(Cc3ccc(-n4c(-c5cccnc5N)nc5ccc(-c6ccc(C7CC7)nc6)nc54)cc3)CC2)n1] | 4-((1-(4-(2-(2-aminopyridin-3-yl)-5-(6- cyclopropylpyridin-3-yl)-3H-imidazo[4,5- b]pyridin-3-yl)benzyl)piperidin-4- yl)amino)pyrimidine-2-carbonitrile |
| 588 | 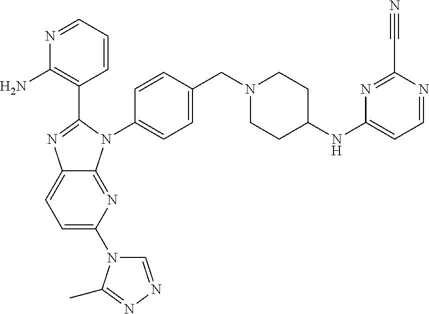 [SMILES: Cc1nncn1-c1ccc2nc(-c3cccnc3N)n(-c3ccc(CN4CCC(Nc5ccnc(C#N)n5)CC4)cc3)c2n1] | 4-((1-(4-(2-(2-aminopyridin-3-yl)-5-(3- methyl-4H-1,2,4-triazol-4-yl)-3H- imidazo[4,5-b]pyridin-3- yl)benzyl)piperidin-4-yl)amino)pyrimidine- 2-carbonitrile |
| 589 | 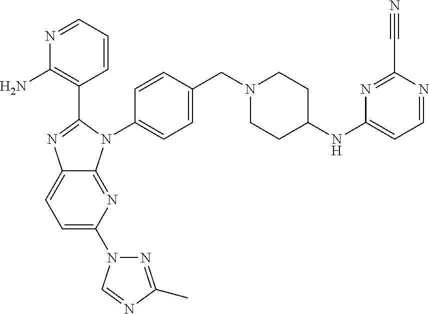 [SMILES: Cc1ncn(-c2ccc3nc(-c4cccnc4N)n(-c4ccc(CN5CCC(Nc6ccnc(C#N)n6)CC5)cc4)c3n2)n1] | 4-((1-(4-(2-(2-aminopyridin-3-yl)-5-(3- methyl-1H-1,2,4-triazol-1-yl)-3H- imidazo[4,5-b]pyridin-3- yl)benzyl)piperidin-4-yl)amino)pyrimidine- 2-carbonitrile |
| 590 | 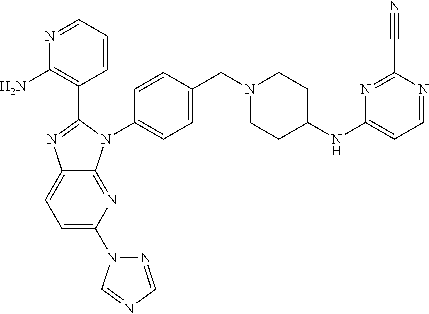 [SMILES: N#Cc1nccc(NC2CCN(Cc3ccc(-n4c(-c5cccnc5N)nc5ccc(-n6cncn6)nc54)cc3)CC2)n1] | 4-((1-(4-(2-(2-aminopyridin-3-yl)-5-(1H- 1,2,4-triazol-1-yl)-3H-imidazo[4,5- b]pyridin-3-yl)benzyl)piperidin-4- yl)amino)pyrimidine-2-carbonitrile |
| 591 | 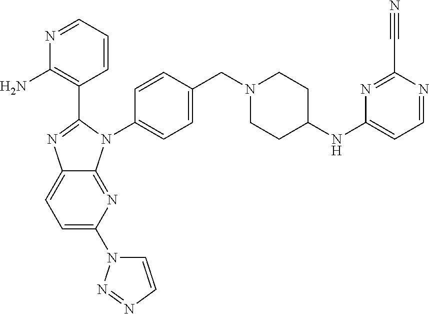 [SMILES: N#Cc1nccc(NC2CCN(Cc3ccc(-n4c(-c5cccnc5N)nc5ccc(-n6ccnn6)nc54)cc3)CC2)n1] | 4-((1-(4-(2-(2-aminopyridin-3-yl)-5-(1H- 1,2,3-triazol-1-yl)-3H-imidazo[4,5- b]pyridin-3-yl)benzyl)piperidin-4- yl)amino)pyrimidine-2-carbonitrile |
| 592 | 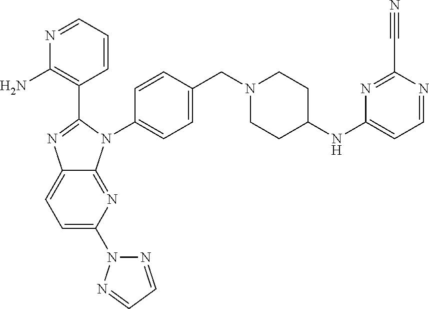 [SMILES: N#Cc1nccc(NC2CCN(Cc3ccc(-n4c(-c5cccnc5N)nc5ccc(-n6nccn6)nc54)cc3)CC2)n1] | 4-((1-(4-(2-(2-aminopyridin-3-yl)-5-(2H- 1,2,3-triazol-2-yl)-3H-imidazo[4,5- b]pyridin-3-yl)benzyl)piperidin-4- yl)amino)pyrimidine-2-carbonitrile |
| 593 | 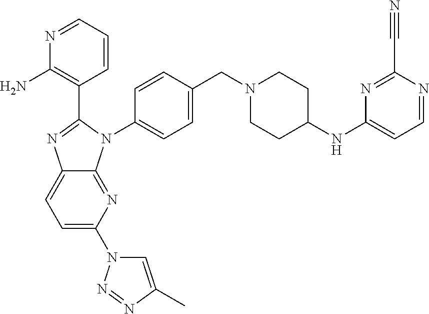 [SMILES: Cc1cn(-c2ccc3nc(-c4cccnc4N)n(-c4ccc(CN5CCC(Nc6ccnc(C#N)n6)CC5)cc4)c3n2)nn1] | 4-((1-(4-(2-(2-aminopyridin-3-yl)-5-(4- methyl-1H-1,2,3-triazol-1-yl)-3H- imidazo[4,5-b]pyridin-3- yl)benzyl)piperidin-4-yl)amino)pyrimidine- 2-carbonitrile |
| 594 | 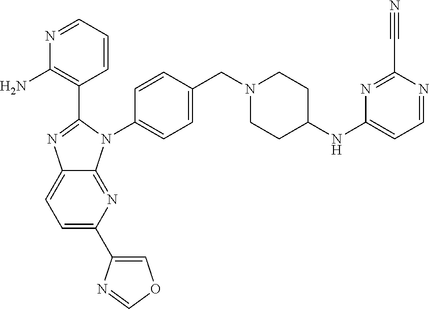 [SMILES: N#Cc1nccc(NC2CCN(Cc3ccc(-n4c(-c5cccnc5N)nc5ccc(-c6cocn6)nc54)cc3)CC2)n1] | 4-((1-(4-(2-(2-aminopyridin-3-yl)-5- (oxazol-4-yl)-3H-imidazo[4,5-b]pyridin-3- yl)benzyl)piperidin-4-yl)amino)pyrimidine- 2-carbonitrile |
| 595 | 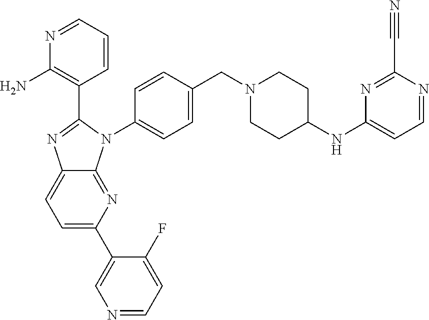 [SMILES: N#Cc1nccc(NC2CCN(Cc3ccc(-n4c(-c5cccnc5N)nc5ccc(-c6cnccc6F)nc54)cc3)CC2)n1] | 4-((1-(4-(2-(2-aminopyridin-3-yl)-5-(4- fluoropyridin-3-yl)-3H-imidazo[4,5- b]pyridin-3-yl)benzyl)piperidin-4- yl)amino)pyrimidine-2-carbonitrile |
| 596 | 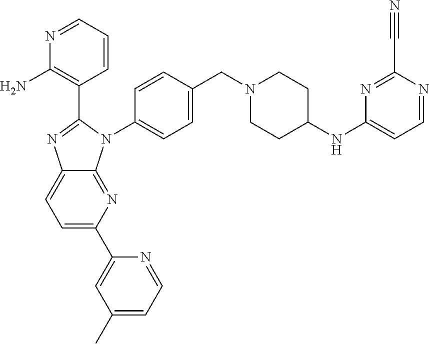 [SMILES: Cc1ccnc(-c2ccc3nc(-c4cccnc4N)n(-c4ccc(CN5CCC(Nc6ccnc(C#N)n6)CC5)cc4)c3n2)c1] | 4-((1-(4-(2-(2-aminopyridin-3-yl)-5-(4- methylpyridin-2-yl)-3H-imidazo[4,5- b]pyridin-3-yl)benzyl)piperidin-4- yl)amino)pyrimidine-2-carbonitrile |
| 597 | 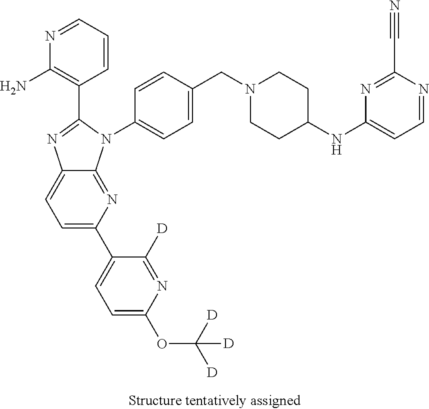 [SMILES: [2H]c1nc(OC([2H])([2H])[2H])ccc1-c1ccc2nc(-c3cccnc3N)n(-c3ccc(CN4CCC(Nc5ccnc(C#N)n5)CC4)cc3)c2n1] | 4-((1-(4-(2-(2-aminopyridin-3-yl)-5-(6- (methoxy-d3)pyridin-3-yl-2-d)-3H- imidazo[4,5-b]pyridin-3- yl)benzyl)piperidin-4-yl)amino)pyrimidine- 2-carbonitrile Mixture of 3D (57.4% and 4D (38.2%) |
| 598 | 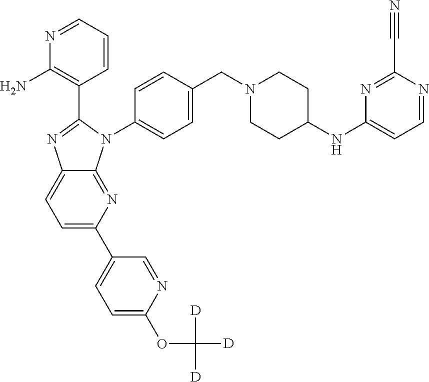 [SMILES: [2H]C([2H])([2H])Oc1ccc(-c2ccc3nc(-c4cccnc4N)n(-c4ccc(CN5CCC(Nc6ccnc(C#N)n6)CC5)cc4)c3n2)cn1] | 4-((1-(4-(2-(2-aminopyridin-3-yl)-5-(6- (methoxy-d3)pyridin-3-yl)-3H-imidazo[4,5- b]pyridin-3-yl)benzyl)piperidin-4- yl)amino)pyrimidine-2-carbonitrile |
| 599 | 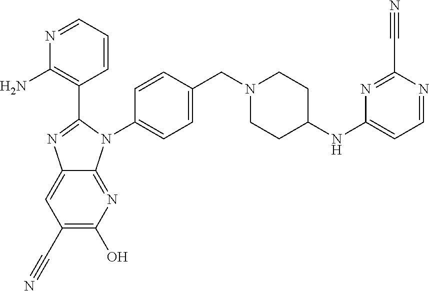 [SMILES: N#Cc1nccc(NC2CCN(Cc3ccc(-n4c(-c5cccnc5N)nc5cc(C#N)c(O)nc54)cc3)CC2)n1] | 2-(2-aminopyridin-3-yl)-3-(4-((4-((2- cyanopyrimidin-4-yl)amino)piperidin-1- yl)methyl)phenyl)-5-hydroxy-3H- imidazo[4,5-b]pyridine-6-carbonitrile |
| 600 | 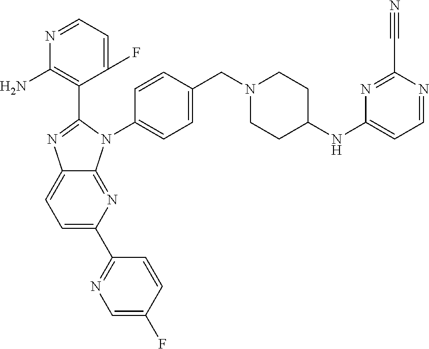 [SMILES: N#Cc1nccc(NC2CCN(Cc3ccc(-n4c(-c5c(F)ccnc5N)nc5ccc(-c6ccc(F)cn6)nc54)cc3)CC2)n1] | 4-((1-(4-(2-(2-amino-4-fluoropyridin-3-yl)- 5-(5-fluoropyridin-2-yl)-3H-imidazo[4,5- b]pyridin-3-yl)benzyl)piperidin-4- yl)amino)pyrimidine-2-carbonitrile |
| 601 | 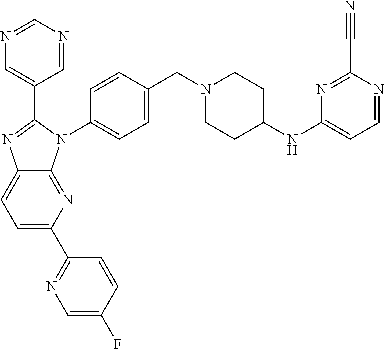 [SMILES: N#Cc1nccc(NC2CCN(Cc3ccc(-n4c(-c5cncnc5)nc5ccc(-c6ccc(F)cn6)nc54)cc3)CC2)n1] | 4-((1-(4-(5-(5-fluoropyridin-2-yl)-2- (pyrimidin-5-yl)-3H-imidazo[4,5-b]pyridin- 3-yl)benzyl)piperidin-4- yl)amino)pyrimidine-2-carbonitrile |
| 602 | 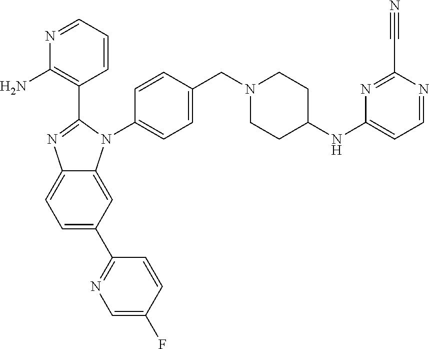 [SMILES: N#Cc1nccc(NC2CCN(Cc3ccc(-n4c(-c5cccnc5N)nc5ccc(-c6ccc(F)cn6)cc54)cc3)CC2)n1] | 4-((1-(4-(2-(2-aminopyridin-3-yl)-6-(5- fluoropyridin-2-yl)-1H-benzo[d]imidazol-1- yl)benzyl)piperidin-4-yl)amino)pyrimidine- 2-carbonitrile |
| 603 | 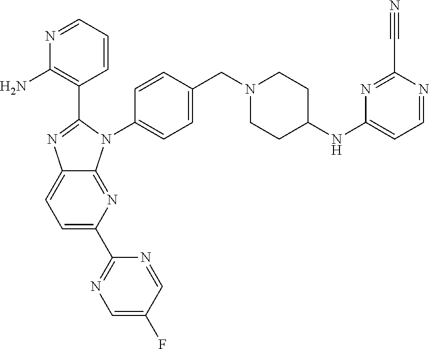 [SMILES: N#Cc1nccc(NC2CCN(Cc3ccc(-n4c(-c5cccnc5N)nc5ccc(-c6ncc(F)cn6)nc54)cc3)CC2)n1] | 4-((1-(4-(2-(2-aminopyridin-3-yl)-5-(5- fluoropyrimidin-2-yl)-3H-imidazo[4,5- b]pyridin-3-yl)benzyl)piperidin-4- yl)amino)pyrimidine-2-carbonitrile |
| 604 | 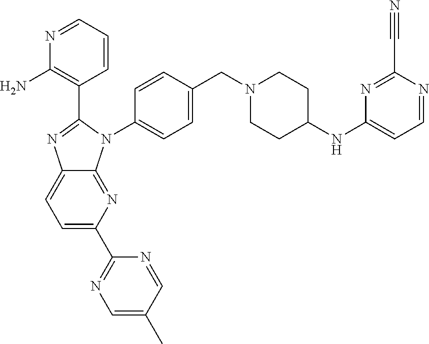 [SMILES: Cc1cnc(-c2ccc3nc(-c4cccnc4N)n(-c4ccc(CN5CCC(Nc6ccnc(C#N)n6)CC5)cc4)c3n2)nc1] | 4-((1-(4-(2-(2-aminopyridin-3-yl)-5-(5- methylpyrimidin-2-yl)-3H-imidazo[4,5- b]pyridin-3-yl)benzyl)piperidin-4- yl)amino)pyrimidine-2-carbonitrile |
| 605 | 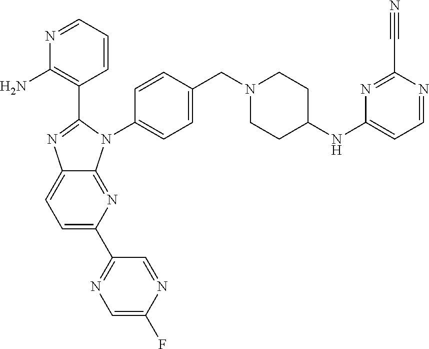 [SMILES: N#Cc1nccc(NC2CCN(Cc3ccc(-n4c(-c5cccnc5N)nc5ccc(-c6cnc(F)cn6)nc54)cc3)CC2)n1] | 4-((1-(4-(2-(2-aminopyridin-3-yl)-5-(5- fluoropyrazin-2-yl)-3H-imidazo[4,5- b]pyridin-3-yl)benzyl)piperidin-4- yl)amino)pyrimidine-2-carbonitrile |
| 606 | 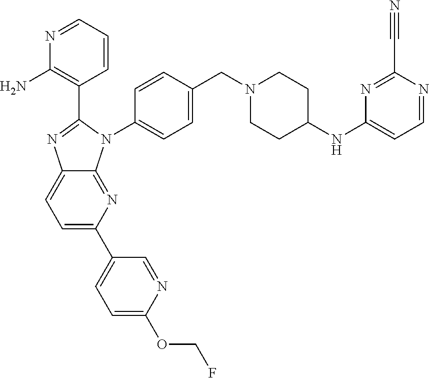 [SMILES: N#Cc1nccc(NC2CCN(Cc3ccc(-n4c(-c5cccnc5N)nc5ccc(-c6ccc(OCF)nc6)nc54)cc3)CC2)n1] | 4-((1- (4-(2-(2-aminopyridin-3-yl)-5-(6- (fluoromethoxy)pyridin-3-yl)-3H- imidazo[4,5-b]pyridin-3- yl)benzyl)piperidin-4-yl)amino)pyrimidine- 2-carbonitrile |
| 607 | 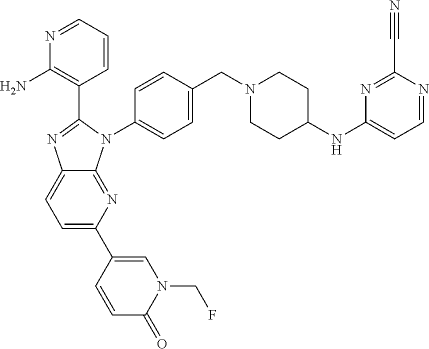 [SMILES: N#Cc1nccc(NC2CCN(Cc3ccc(-n4c(-c5cccnc5N)nc5ccc(-c6ccc(=O)n(CF)c6)nc54)cc3)CC2)n1] | 4-((1-(4-(2-(2-aminopyridin-3-yl)-5-(1- (fluoromethyl)-6-oxo-1,6-dihydropyridin-3- yl)-3H-imidazo[4,5-b]pyridin-3- yl)benzyl)piperidin-4-yl)amino)pyrimidine- 2-carbonitrile |
| 608 | 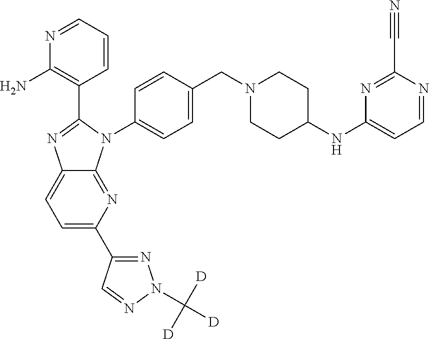 [SMILES: [2H]C([2H])([2H])n1ncc(-c2ccc3nc(-c4cccnc4N)n(-c4ccc(CN5CCC(Nc6ccnc(C#N)n6)CC5)cc4)c3n2)n1] | 4-((1-(4-(2-(2-aminopyridin-3-yl)-5-(2- (methyl-d3)-2H-1,2,3-triazol-4-yl)-3H- imidazo[4,5-b]pyridin-3- yl)benzyl)piperidin-4-yl)amino)pyrimidine- 2-carbonitrile |
| 609 | 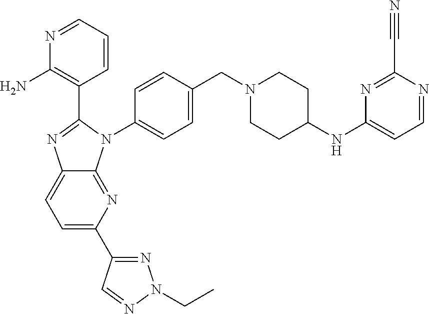 [SMILES: CCn1ncc(-c2ccc3nc(-c4cccnc4N)n(-c4ccc(CN5CCC(Nc6ccnc(C#N)n6)CC5)cc4)c3n2)n1] | 4-((1-(4-(2-(2-aminopyridin-3-yl)-5-(2- ethyl-2H-1,2,3-triazol-4-yl)-3H- imidazo[4,5-b]pyridin-3- yl)benzyl)piperidin-4-yl)amino)pyrimidine- 2-carbonitrile |
| 610 | 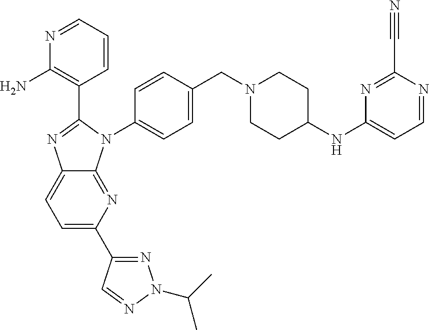 [SMILES: CC(C)n1ncc(-c2ccc3nc(-c4cccnc4N)n(-c4ccc(CN5CCC(Nc6ccnc(C#N)n6)CC5)cc4)c3n2)n1] | 4-((1-(4-(2-(2-aminopyridin-3-yl)-5-(2- isopropyl-2H-1,2,3-triazol-4-yl)-3H- imidazo[4,5-b]pyridin-3- yl)benzyl)piperidin-4-yl)amino)pyrimidine- 2-carbonitrile |
| 611 | 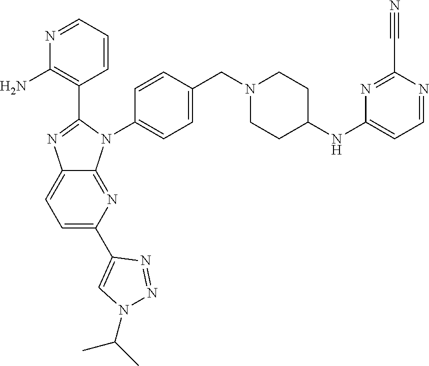 [SMILES: CC(C)n1cc(-c2ccc3nc(-c4cccnc4N)n(-c4ccc(CN5CCC(Nc6ccnc(C#N)n6)CC5)cc4)c3n2)nn1] | 4-((1-(4-(2-(2-aminopyridin-3-yl)-5-(1- isopropyl-1H-1,2,3-triazol-4-yl)-3H- imidazo[4,5-b]pyridin-3- yl)benzyl)piperidin-4-yl)amino)pyrimidine- 2-carbonitrile |
| 612 | 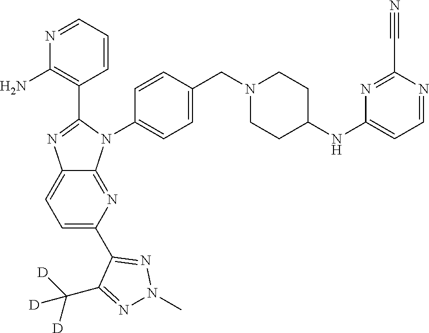 [SMILES: [2H]C([2H])([2H])c1nn(C)nc1-c1ccc2nc(-c3cccnc3N)n(-c3ccc(CN4CCC(Nc5ccnc(C#N)n5)CC4)cc3)c2n1] | 4-((1-(4-(2-(2-aminopyridin-3-yl)-5-(2- methyl-5-(methyl-d3)-2H-1,2,3-triazol-4- yl)-3H-imidazo[4,5-b]pyridin-3- yl)benzyl)piperidin-4-yl)amino)pyrimidine- 2-carbonitrile |
| 613 | 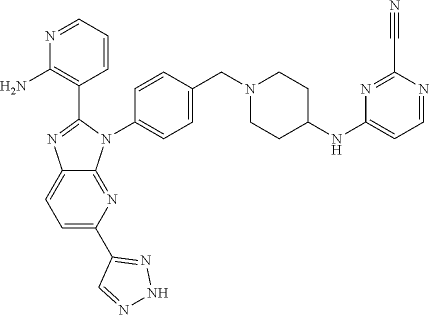 [SMILES: N#Cc1nccc(NC2CCN(Cc3ccc(-n4c(-c5cccnc5N)nc5ccc(-c6cn[nH]n6)nc54)cc3)CC2)n1] | 4-((1-(4-(2-(2-aminopyridin-3-yl)-5-(2H- 1,2,3-triazol-4-yl)-3H-imidazo[4,5- b]pyridin-3-yl)benzyl)piperidin-4- yl)amino)pyrimidine-2-carbonitrile |
| 614 | 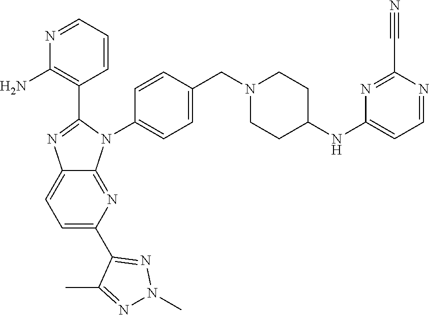 [SMILES: Cc1nn(C)nc1-c1ccc2nc(-c3cccnc3N)n(-c3ccc(CN4CCC(Nc5ccnc(C#N)n5)CC4)cc3)c2n1] | 4-((1-(4-(2-(2-aminopyridin-3-yl)-5-(2,5- dimethyl-2H-1,2,3-triazol-4-yl)-3H- imidazo[4,5-b]pyridin-3- yl)benzyl)piperidin-4-yl)amino)pyrimidine- 2-carbonitrile |
| 615 | 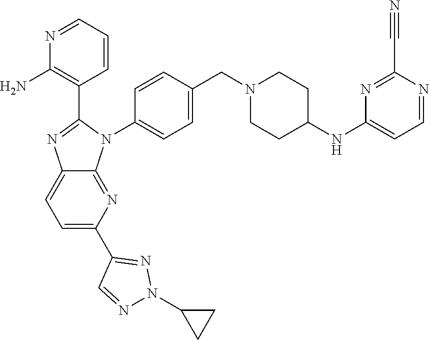 [SMILES: N#Cc1nccc(NC2CCN(Cc3ccc(-n4c(-c5cccnc5N)nc5ccc(-c6cnn(C7CC7)n6)nc54)cc3)CC2)n1] | 4-((1-(4-(2-(2-aminopyridin-3-yl)-5-(2- cyclopropyl-2H-1,2,3-triazol-4-yl)-3H- imidazo[4,5-b]pyridin-3- yl)benzyl)piperidin-4-yl)amino)pyrimidine- 2-carbonitrile |
| 616 | 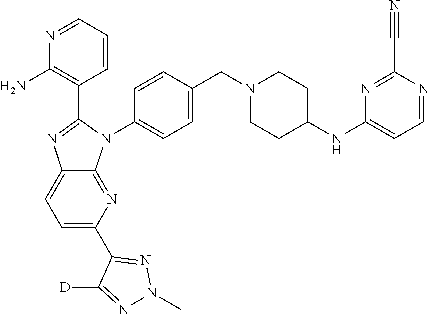 [SMILES: [2H]c1nn(C)nc1-c1ccc2nc(-c3cccnc3N)n(-c3ccc(CN4CCC(Nc5ccnc(C#N)n5)CC4)cc3)c2n1] | 4-((1-(4-(2-(2-aminopyridin-3-yl)-5-(2- methyl-2H-1,2,3-triazol-4-yl-5-d)-3H- imidazo[4,5-b]pyridin-3- yl)benzyl)piperidin-4-yl)amino)pyrimidine- 2-carbonitrile |
| 617 | 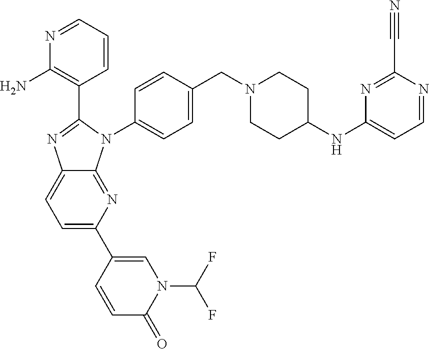 [SMILES: N#Cc1nccc(NC2CCN(Cc3ccc(-n4c(-c5cccnc5N)nc5ccc(-c6ccc(=O)n(C(F)F)c6)nc54)cc3)CC2)n1] | 4-((1-(4-(2-(2-aminopyridin-3-yl)-5-(1- (difluoromethyl)-6-oxo-1,6-dihydropyridin- 3-yl)-3H-imidazo[4,5-b]pyridin-3- yl)benzyl)piperidin-4-yl)amino)pyrimidine- 2-carbonitrile |
| 618 | 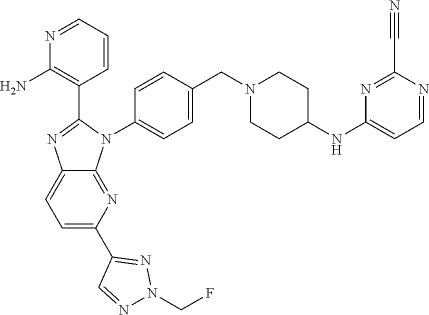 [SMILES: N#Cc1nccc(NC2CCN(Cc3ccc(-n4c(-c5cccnc5N)nc5ccc(-c6cnn(CF)n6)nc54)cc3)CC2)n1] | 4-((1-(4-(2-(2-aminopyridin-3-yl)-5-(2- (fluoromethyl)-2H-1,2,3-triazol-4-yl)-3H- imidazo[4,5-b]pyridin-3- yl)benzyl)piperidin-4-yl)amino)pyrimidine- 2-carbonitrile |
| 619 | 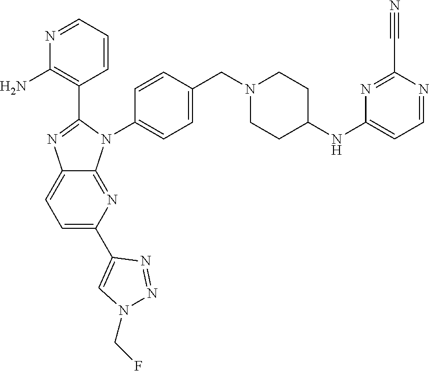 [SMILES: N#Cc1nccc(NC2CCN(Cc3ccc(-n4c(-c5cccnc5N)nc5ccc(-c6cn(CF)nn6)nc54)cc3)CC2)n1] | 4-((1-(4-(2-(2-aminopyridin-3-yl)-5-(1- (fluoromethyl)-1H-1,2,3-triazol-4-yl)-3H- imidazo[4,5-b]pyridin-3- yl)benzyl)piperidin-4-yl)amino)pyrimidine- 2-carbonitrile |
| 620 | 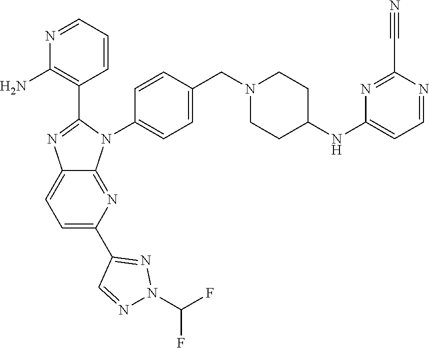 [SMILES: N#Cc1nccc(NC2CCN(Cc3ccc(-n4c(-c5cccnc5N)nc5ccc(-c6cnn(C(F)F)n6)nc54)cc3)CC2)n1] | 4-((1-(4-(2-(2-aminopyridin-3-yl)-5-(2- (difluoromethyl)-2H-1,2,3-triazol-4-yl)-3H- imidazo[4,5-b]pyridin-3- yl)benzyl)piperidin-4-yl)amino)pyrimidine- 2-carbonitrile |
| 621 | 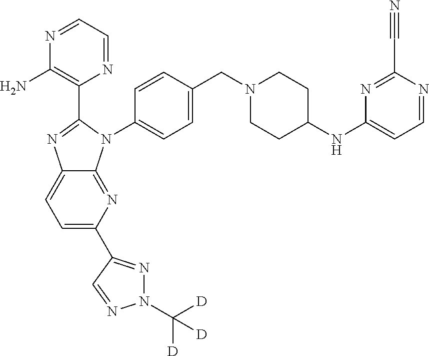 [SMILES: [2H]C([2H])([2H])n1ncc(-c2ccc3nc(-c4nccnc4N)n(-c4ccc(CN5CCC(Nc6ccnc(C#N)n6)CC5)cc4)c3n2)n1] | 4-((1-(4-(2-(3-aminopyrazin-2-yl)-5-(2- (methyl-d3)-2H-1,2,3-triazol-4-yl)-3H- imidazo[4,5-b]pyridin-3- yl)benzyl)piperidin-4-yl)amino)pyrimidine- 2-carbonitrile |
| 622 | 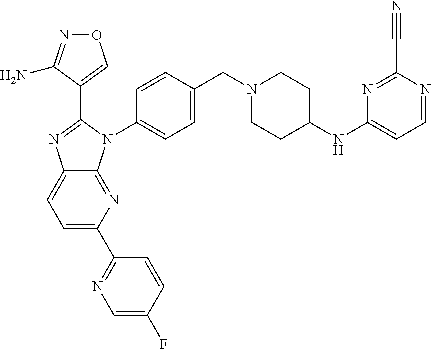 [SMILES: N#Cc1nccc(NC2CCN(Cc3ccc(-n4c(-c5conc5N)nc5ccc(-c6ccc(F)cn6)nc54)cc3)CC2)n1] | 4-((1-(4-(2-(3-aminoisoxazol-4-yl)-5-(5- fluoropyridin-2-yl)-3H-imidazo[4,5- b]pyridin-3-yl)benzyl)piperidin-4- yl)amino)pyrimidine-2-carbonitrile |
| 623 | 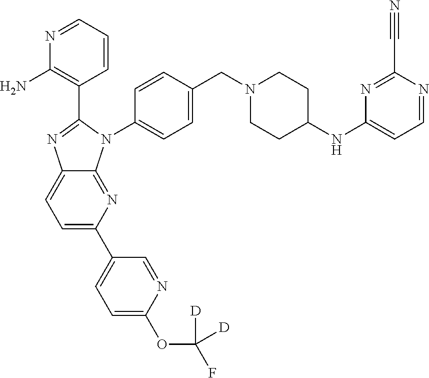 [SMILES: [2H]C([2H])(F)Oc1ccc(-c2ccc3nc(-c4cccnc4N)n(-c4ccc(CN5CCC(Nc6ccnc(C#N)n6)CC5)cc4)c3n2)cn1] | 4-((1-(4-(2-(2-aminopyridin-3-yl)-5-(6- (fluoromethoxy-d2)pyridin-3-yl)-3H- imidazo[4,5-b]pyridin-3- yl)benzyl)piperidin-4-yl)amino)pyrimidine- 2-carbonitrile |
| 624 | 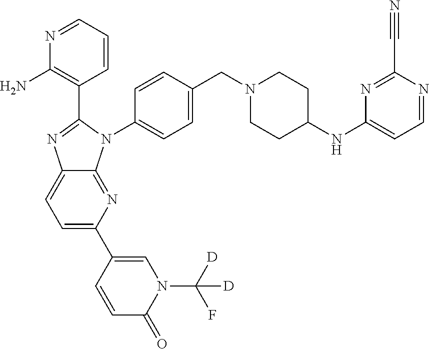 [SMILES: [2H]C([2H])(F)n1cc(-c2ccc3nc(-c4cccnc4N)n(-c4ccc(CN5CCC(Nc6ccnc(C#N)n6)CC5)cc4)c3n2)ccc1=O] | 4-((1-(4-(2-(2-aminopyridin-3-yl)-5-(1- (fluoromethyl-d2)-6-oxo-1,6- dihydropyridin-3-yl)-3H-imidazo[4,5- b]pyridin-3-yl)benzyl)piperidin-4- yl)amino)pyrimidine-2-carbonitrile |
| 625 | 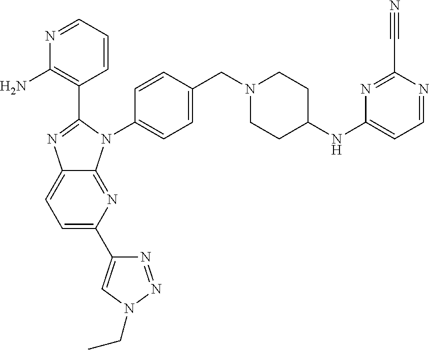 [SMILES: CCn1cc(-c2ccc3nc(-c4cccnc4N)n(-c4ccc(CN5CCC(Nc6ccnc(C#N)n6)CC5)cc4)c3n2)nn1] | 4-((1-(4-(2-(2-aminopyridin-3-yl)-5-(1- ethyl-1H-1,2,3-triazol-4-yl)-3H- imidazo[4,5-b]pyridin-3- yl)benzyl)piperidin-4-yl)amino)pyrimidine- 2-carbonitrile |
| 626 | 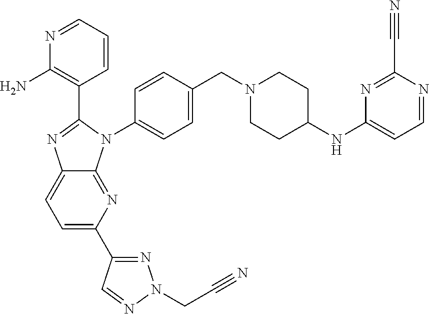 [SMILES: N#CCn1ncc(-c2ccc3nc(-c4cccnc4N)n(-c4ccc(CN5CCC(Nc6ccnc(C#N)n6)CC5)cc4)c3n2)n1] | 4-((1-(4-(2-(2-aminopyridin-3-yl)-5-(2- (cyanomethyl)-2H-1,2,3-triazol-4-yl)-3H- imidazo[4,5-b]pyridin-3- yl)benzyl)piperidin-4-yl)amino)pyrimidine- 2-carbonitrile |
| 627 | 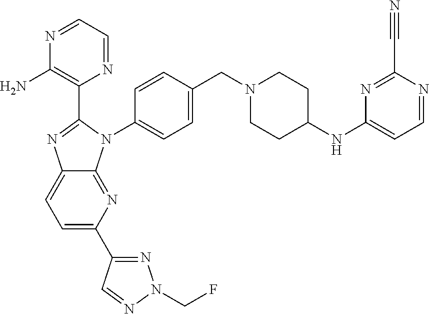 [SMILES: N#Cc1nccc(NC2CCN(Cc3ccc(-n4c(-c5nccnc5N)nc5ccc(-c6cnn(CF)n6)nc54)cc3)CC2)n1] | 4-((1-(4-(2-(3-aminopyrazin-2-yl)-5-(2- (fluoromethyl)-2H-1,2,3-triazol-4-yl)-3H- imidazo[4,5-b]pyridin-3- yl)benzyl)piperidin-4-yl)amino)pyrimidine- 2-carbonitrile |
| 628 | 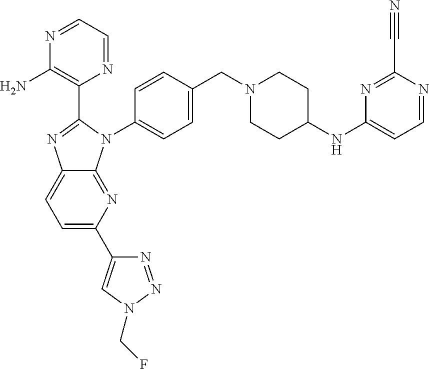 [SMILES: N#Cc1nccc(NC2CCN(Cc3ccc(-n4c(-c5nccnc5N)nc5ccc(-c6cn(CF)nn6)nc54)cc3)CC2)n1] | 4-((1-(4-(2-(3-aminopyrazin-2-yl)-5-(1- (fluoromethyl)-1H-1,2,3-triazol-4-yl)-3H- imidazo[4,5-b]pyridin-3- yl)benzyl)piperidin-4-yl)amino)pyrimidine- 2-carbonitrile |
| 629 | 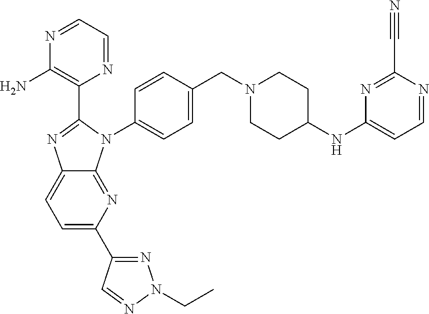 [SMILES: CCn1ncc(-c2ccc3nc(-c4nccnc4N)n(-c4ccc(CN5CCC(Nc6ccnc(C#N)n6)CC5)cc4)c3n2)n1] | 4-((1-(4-(2-(3-aminopyrazin-2-yl)-5-(2- ethyl-2H-1,2,3-triazol-4-yl)-3H- imidazo[4,5-b]pyridin-3- yl)benzyl)piperidin-4-yl)amino)pyrimidine- 2-carbonitrile |
| 630 | 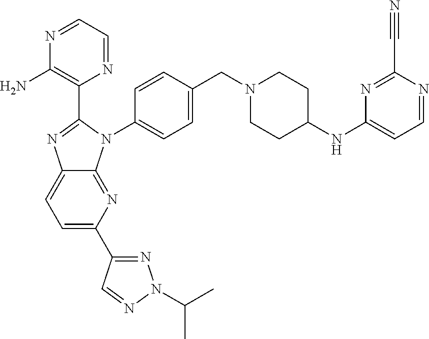 [SMILES: CC(C)n1ncc(-c2ccc3nc(-c4nccnc4N)n(-c4ccc(CN5CCC(Nc6ccnc(C#N)n6)CC5)cc4)c3n2)n1] | 4-((1-(4-(2-(3-aminopyrazin-2-yl)-5-(2- isopropyl-2H-1,2,3-triazol-4-yl)-3H- imidazo[4,5-b ]pyridin-3- yl)benzyl)piperidin-4-yl)amino)pyrimidine- 2-carbonitrile |
| 631 | 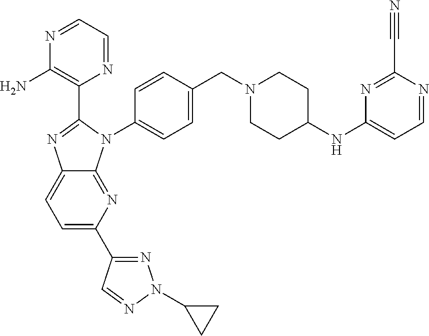 [SMILES: N#Cc1nccc(NC2CCN(Cc3ccc(-n4c(-c5nccnc5N)nc5ccc(-c6cnn(C7CC7)n6)nc54)cc3)CC2)n1] | 4-((1- (4-(2-(3-aminopyrazin-2-yl)-5-(2- cyclopropyl-2H-1,2,3-triazol-4-yl)-3H- imidazo[4,5-b]pyridin-3- yl)benzyl)piperidin-4-yl)amino)pyrimidine- 2-carbonitrile |
| 632 | 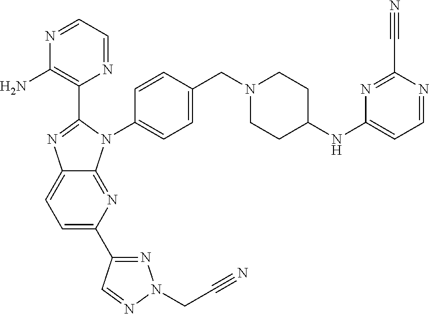 [SMILES: N#CCn1ncc(-c2ccc3nc(-c4nccnc4N)n(-c4ccc(CN5CCC(Nc6ccnc(C#N)n6)CC5)cc4)c3n2)n1] | 4-((1- (4-(2-(3-aminopyrazin-2-yl)-5-(2- (cyanomethyl)-2H-1,2,3-triazol-4-yl)-3H- imidazo[4,5-b]pyridin-3- yl)benzyl)piperidin-4-yl)amino)pyrimidine- 2-carbonitrile |
| 633 | 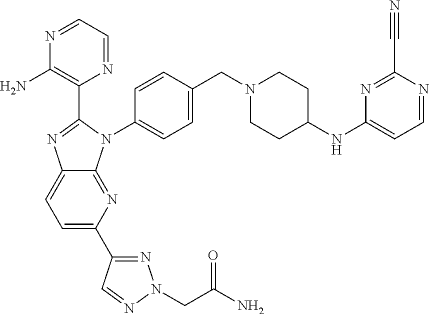 [SMILES: N#Cc1nccc(NC2CCN(Cc3ccc(-n4c(-c5nccnc5N)nc5ccc(-c6cnn(CC(N)=O)n6)nc54)cc3)CC2)n1] | 2-(4-(2-(3-aminopyrazin-2-yl)-3-(4-((4-((2- cyanopyrimidin-4-yl)amino)piperidin-1- yl)methyl)phenyl)-3H-imidazo[4,5- b]pyridin-5-yl)-2H-1,2,3-triazol-2- yl)acetamide |
| 634 | 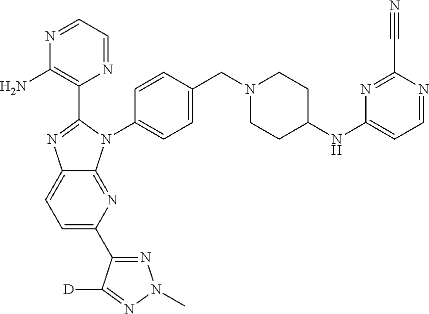 [SMILES: [2H]c1nn(C)nc1-c1ccc2nc(-c3nccnc3N)n(-c3ccc(CN4CCC(Nc5ccnc(C#N)n5)CC4)cc3)c2n1] | 4-((1-(4-(2-(3-aminopyrazin-2-yl)-5-(2- methyl-2H-1,2,3-triazol-4-yl-5-d)-3H- imidazo[4,5-b]pyridin-3- yl)benzyl)piperidin-4-yl)amino)pyrimidine- 2-carbonitrile |
| 635 | 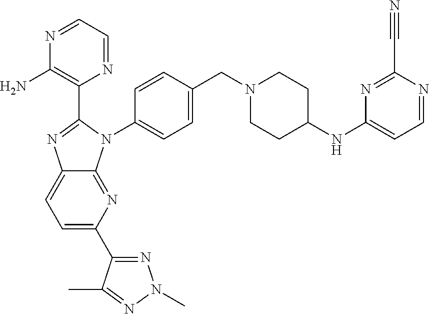 [SMILES: Cc1nn(C)nc1-c1ccc2nc(-c3nccnc3N)n(-c3ccc(CN4CCC(Nc5ccnc(C#N)n5)CC4)cc3)c2n1] | 4-((1-(4-(2-(3-aminopyrazin-2-yl)-5-(2,5- dimethyl-2H-1,2,3-triazol-4-yl)-3H- imidazo[4,5-b]pyridin-3- yl)benzyl)piperidin-4-yl)amino)pyrimidine- 2-carbonitrile |
| 636 | 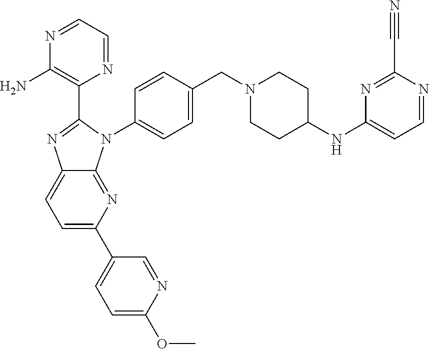 [SMILES: COc1ccc(-c2ccc3nc(-c4nccnc4N)n(-c4ccc(CN5CCC(Nc6ccnc(C#N)n6)CC5)cc4)c3n2)cn1] | 4-((1-(4-(2-(3-aminopyrazin-2-yl)-5-(6- methoxypyridin-3-yl)-3H-imidazo[4,5- b]pyridin-3-yl)benzyl)piperidin-4- yl)amino)pyrimidine-2-carbonitrile |
| 637 | 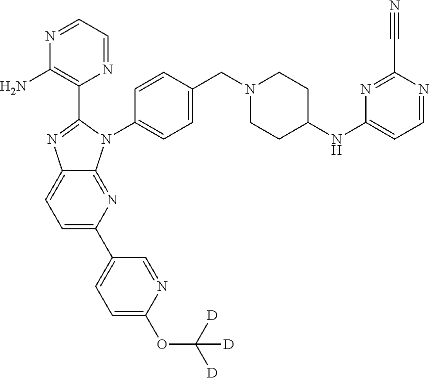 [SMILES: [2H]C([2H])([2H])Oc1ccc(-c2ccc3nc(-c4nccnc4N)n(-c4ccc(CN5CCC(Nc6ccnc(C#N)n6)CC5)cc4)c3n2)cn1] | 4-((1-(4-(2-(3-aminopyrazin-2-yl)-5-(6- (methoxy-d3)pyridin-3-yl)-3H-imidazo[4,5- b]pyridin-3-yl)benzyl)piperidin-4- yl)amino)pyrimidine-2-carbonitrile |
| 638 | 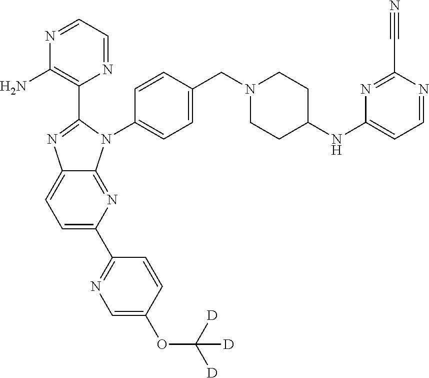 [SMILES: [2H]C([2H])([2H])Oc1ccc(-c2ccc3nc(-c4nccnc4N)n(-c4ccc(CN5CCC(Nc6ccnc(C#N)n6)CC5)cc4)c3n2)nc1] | 4-((1-(4-(2-(3-aminopyrazin-2-yl)-5-(5- (methoxy-d3)pyridin-2-yl)-3H-imidazo[4,5- b]pyridin-3-yl)benzyl)piperidin-4- yl)amino)pyrimidine-2-carbonitrile |
| 639 | 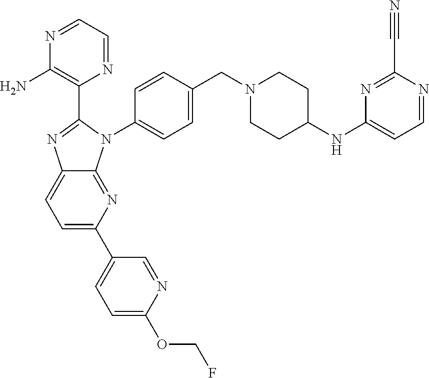 [SMILES: N#Cc1nccc(NC2CCN(Cc3ccc(-n4c(-c5nccnc5N)nc5ccc(-c6ccc(OCF)nc6)nc54)cc3)CC2)n1] | 4-((1-(4-(2-(3-aminopyrazin-2-yl)-5-(6- (fluoromethoxy)pyridin-3-yl)-3H- imidazo[4,5-b]pyridin-3- yl)benzyl)piperidin-4-yl)amino)pyrimidine- 2-carbonitrile |
| 640 | 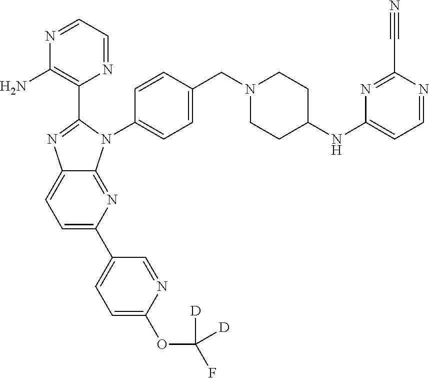 [SMILES: [2H]C([2H])(F)Oc1ccc(-c2ccc3nc(-c4nccnc4N)n(-c4ccc(CN5CCC(Nc6ccnc(C#N)n6)CC5)cc4)c3n2)cn1] | 4-((1-(4-(2-(3-aminopyrazin-2-yl)-5-(6- (fluoromethoxy-d2)pyridin-3-yl)-3H- imidazo[4,5-b]pyridin-3- yl)benzyl)piperidin-4-yl)amino)pyrimidine- 2-carbonitrile |
| 641 | 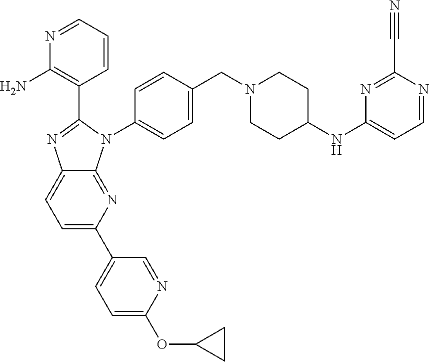 [SMILES: N#Cc1nccc(NC2CCN(Cc3ccc(-n4c(-c5cccnc5N)nc5ccc(-c6ccc(OC7CC7)nc6)nc54)cc3)CC2)n1] | 4-((1-(4-(2-(2-aminopyridin-3-yl)-5-(6- cyclopropoxypyridin-3-yl)-3H-imidazo[4,5- b]pyridin-3-yl)benzyl)piperidin-4- yl)amino)pyrimidine-2-carbonitrile |
| 642 | 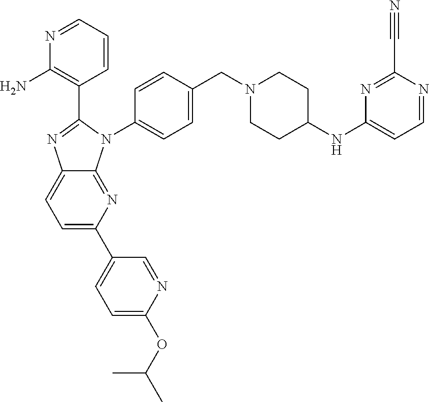 [SMILES: CC(C)Oc1ccc(-c2ccc3nc(-c4cccnc4N)n(-c4ccc(CN5CCC(Nc6ccnc(C#N)n6)CC5)cc4)c3n2)cn1] | 4-((1-(4-(2-(2-aminopyridin-3-yl)-5-(6- isopropoxypyridin-3-yl)-3H-imidazo[4,5- b]pyridin-3-yl)benzyl)piperidin-4- yl)amino)pyrimidine-2-carbonitrile |
| 643 | 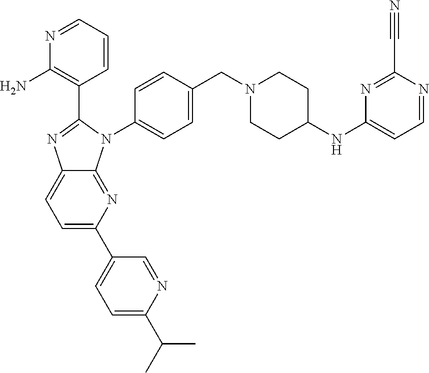 [SMILES: CC(C)c1ccc(-c2ccc3nc(-c4cccnc4N)n(-c4ccc(CN5CCC(Nc6ccnc(C#N)n6)CC5)cc4)c3n2)cn1] | 4-((1-(4-(2-(2-aminopyridin-3-yl)-5-(6- isopropylpyridin-3-yl)-3H-imidazo[4,5- b]pyridin-3-yl)benzyl)piperidin-4- yl)amino)pyrimidine-2-carbonitrile |
| 644 | 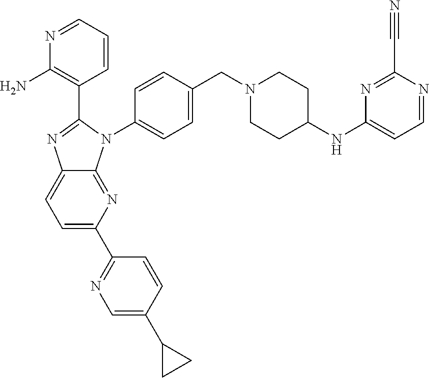 [SMILES: N#Cc1nccc(NC2CCN(Cc3ccc(-n4c(-c5cccnc5N)nc5ccc(-c6ccc(C7CC7)cn6)nc54)cc3)CC2)n1] | 4-((1-(4-(2-(2-aminopyridin-3-yl)-5-(5- cyclopropylpyridin-2-yl)-3H-imidazo[4,5- b]pyridin-3-yl)benzyl)piperidin-4- yl)amino)pyrimidine-2-carbonitrile |
| 645 | 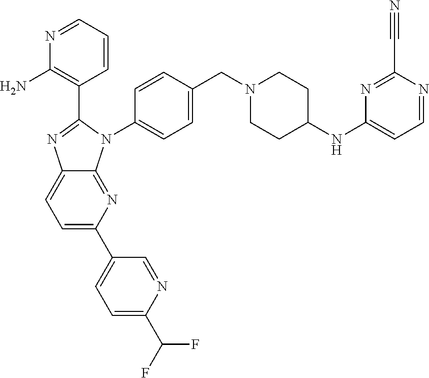 [SMILES: N#Cc1nccc(NC2CCN(Cc3ccc(-n4c(-c5cccnc5N)nc5ccc(-c6ccc(C(F)F)nc6)nc54)cc3)CC2)n1] | 4-((1-(4-(2-(2-aminopyridin-3-yl)-5-(6- (difluoromethyl)pyridin-3-yl)-3H- imidazo[4,5-b]pyridin-3- yl)benzyl)piperidin-4-yl)amino)pyrimidine- 2-carbonitrile |
| 646 | 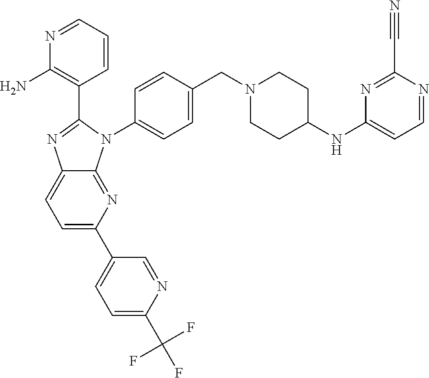 [SMILES: N#Cc1nccc(NC2CCN(Cc3ccc(-n4c(-c5cccnc5N)nc5ccc(-c6ccc(C(F)(F)F)nc6)nc54)cc3)CC2)n1] | 4-((1-(4-(2-(2-aminopyridin-3-yl)-5-(6- (trifluoromethyl)pyridin-3-yl)-3H- imidazo[4,5-b]pyridin-3- yl)benzyl)piperidin-4-yl)amino)pyrimidine- 2-carbonitrile |
| 647 | 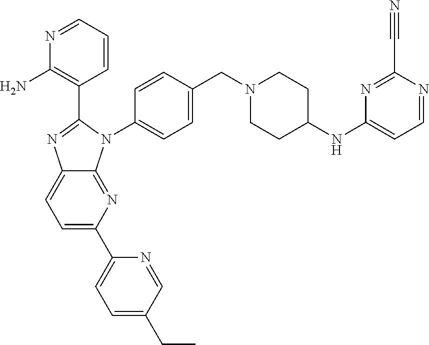 [SMILES: CCc1ccc(-c2ccc3nc(-c4cccnc4N)n(-c4ccc(CN5CCC(Nc6ccnc(C#N)n6)CC5)cc4)c3n2)nc1] | 4-((1-(4-(2-(2-aminopyridin-3-yl)-5-(5- ethylpyridin-2-yl)-3H-imidazo[4,5- b]pyridin-3-yl)benzyl)piperidin-4- yl)amino)pyrimidine-2-carbonitrile |
| 648 | 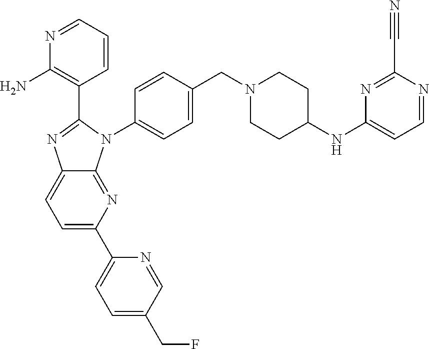 [SMILES: N#Cc1nccc(NC2CCN(Cc3ccc(-n4c(-c5cccnc5N)nc5ccc(-c6ccc(CF)cn6)nc54)cc3)CC2)n1] | 4-((1-(4-(2-(2-aminopyridin-3-yl)-5-(5- (fluoromethyl)pyridin-2-yl)- 3H- imidazo[4,5-b]pyridin-3- yl)benzyl)piperidin-4-yl)amino)pyrimidine- 2-carbonitrile |
| 649 | 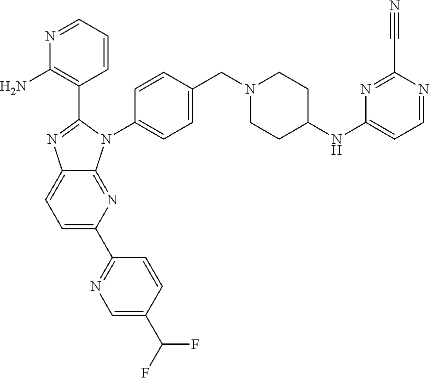 [SMILES: N#Cc1nccc(NC2CCN(Cc3ccc(-n4c(-c5cccnc5N)nc5ccc(-c6ccc(C(F)F)cn6)nc54)cc3)CC2)n1] | 4-((1-(4-(2-(2-aminopyridin-3-yl)-5-(5- (difluoromethyl)pyridin-2-yl)-3H- imidazo[4,5-b]pyridin-3- yl)benzyl)piperidin-4-yl)amino)pyrimidine- 2-carbonitrile |
| 650 | 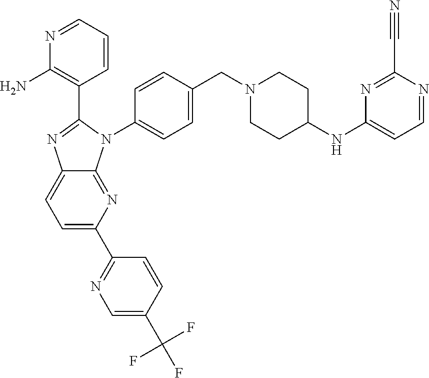 [SMILES: N#Cc1nccc(NC2CCN(Cc3ccc(-n4c(-c5cccnc5N)nc5ccc(-c6ccc(C(F)(F)F)cn6)nc54)cc3)CC2)n1] | 4-((1-(4-(2-(2-aminopyridin-3-yl)-5-(5- (trifluoromethyl)pyridin-2-yl)-3H- imidazo[4,5-b]pyridin-3- yl)benzyl)piperidin-4-yl)amino)pyrimidine- 2-carbonitrile |

Another embodiment provides an AKT1 inhibitory compound, or a pharmaceutically acceptable salt or solvate thereof, having a structure presented in Table 2.

TABLE 2

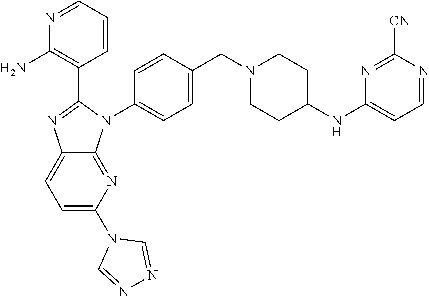 [SMILES: N#Cc1nccc(NC2CCN(Cc3ccc(-n4c(-c5cccnc5N)nc5ccc(-n6cnnc6)nc54)cc3)CC2)n1]

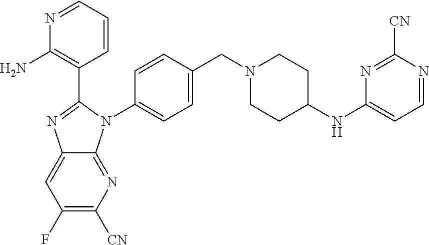 [SMILES: N#Cc1nccc(NC2CCN(Cc3ccc(-n4c(-c5cccnc5N)nc5cc(F)c(C#N)nc54)cc3)CC2)n1]

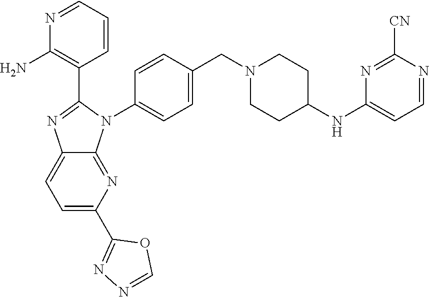 [SMILES: N#Cc1nccc(NC2CCN(Cc3ccc(-n4c(-c5cccnc5N)nc5ccc(-c6nnco6)nc54)cc3)CC2)n1]

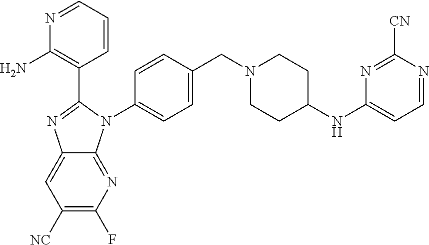 [SMILES: N#Cc1nccc(NC2CCN(Cc3ccc(-n4c(-c5cccnc5N)nc5cc(C#N)c(F)nc54)cc3)CC2)n1]

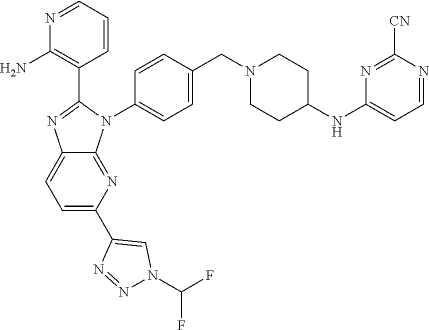 [SMILES: N#Cc1nccc(NC2CCN(Cc3ccc(-n4c(-c5cccnc5N)nc5ccc(-c6cn(C(F)F)nn6)nc54)cc3)CC2)n1]

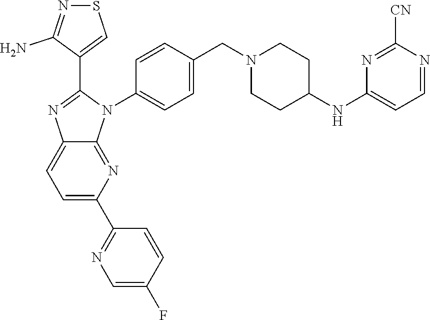 [SMILES: N#Cc1nccc(NC2CCN(Cc3ccc(-n4c(-c5csnc5N)nc5ccc(-c6ccc(F)cn6)nc54)cc3)CC2)n1]

### Preparation of Compounds

The compounds used in the synthetic chemistry reactions described herein are made according to organic synthesis techniques known to those skilled in this art, starting from commercially available chemicals and/or from compounds described in the chemical literature. “Commercially available chemicals” are obtained from standard commercial sources including Acros Organics (Pittsburgh, PA), Aldrich Chemical (Milwaukee, WI, including Sigma Chemical and Fluka), Apin Chemicals Ltd. (Milton Park, UK), Avocado Research (Lancashire, U.K.), BDH Inc. (Toronto, Canada), Bionet (Cornwall, U.K.), Chemservice Inc. (West Chester, PA), Crescent Chemical Co. (Hauppauge, NY), Eastman Organic Chemicals, Eastman Kodak Company (Rochester, NY), Fisher Scientific Co. (Pittsburgh, PA), Fisons Chemicals (Leicestershire, UK), Frontier Scientific (Logan, UT), ICN Biomedicals, Inc. (Costa Mesa, CA), Key Organics (Cornwall, U.K.), Lancaster Synthesis (Windham, NH), Maybridge Chemical Co. Ltd. (Cornwall, U.K.), Parish Chemical Co. (Orem, UT), Pfaltz & Bauer, Inc. (Waterbury, CN), Polyorganix (Houston, TX), Pierce Chemical Co. (Rockford, IL), Riedel de Haen AG (Hanover, Germany), Spectrum Quality Product, Inc. (New Brunswick, NJ), TCI America (Portland, OR), Trans World Chemicals, Inc. (Rockville, MD), and Wako Chemicals USA, Inc. (Richmond, VA).

Suitable reference books and treatise that detail the synthesis of reactants useful in the preparation of compounds described herein, or provide references to articles that describe the preparation, include for example, “Synthetic Organic Chemistry”, John Wiley & Sons, Inc., New York; S. R. Sandler et al., “Organic Functional Group Preparations,” 2nd Ed., Academic Press, New York, 1983; H. O. House, “Modern Synthetic Reactions”, 2nd Ed., W. A. Benjamin, Inc. Menlo Park, Calif. 1972; T. L. Gilchrist, “Heterocyclic Chemistry”, 2nd Ed., John Wiley & Sons, New York, 1992; J. March, “Advanced Organic Chemistry: Reactions, Mechanisms and Structure”, 4th Ed., Wiley-Interscience, New York, 1992. Additional suitable reference books and treatise that detail the synthesis of reactants useful in the preparation of compounds described herein, or provide references to articles that describe the preparation, include for example, Fuhrhop, J. and Penzlin G. “Organic Synthesis: Concepts, Methods, Starting Materials”, Second, Revised and Enlarged Edition (1994) John Wiley & Sons ISBN: 3-527-29074-5; Hoffman, R. V. “Organic Chemistry, An Intermediate Text” (1996) Oxford University Press, ISBN 0-19-509618-5; Larock, R. C. “Comprehensive Organic Transformations: A Guide to Functional Group Preparations” 2nd Edition (1999) Wiley-VCH, ISBN: 0-471-19031-4; March, J. “Advanced Organic Chemistry: Reactions, Mechanisms, and Structure” 4th Edition (1992) John Wiley & Sons, ISBN: 0-471-60180-2; Otera, J. (editor) “Modern Carbonyl Chemistry” (2000) Wiley-VCH, ISBN: 3-527-29871-1; Patai, S. “Patai's 1992 Guide to the Chemistry of Functional Groups” (1992) Interscience ISBN: 0-471-93022-9; Solomons, T. W. G. “Organic Chemistry” 7th Edition (2000) John Wiley & Sons, ISBN: 0-471-19095-0; Stowell, J. C., “Intermediate Organic Chemistry” 2nd Edition (1993) Wiley-Interscience, ISBN: 0-471-57456-2; “Industrial Organic Chemicals: Starting Materials and Intermediates: An Ullmann's Encyclopedia” (1999) John Wiley & Sons, ISBN: 3-527-29645-X, in 8 volumes; “Organic Reactions” (1942-2000) John Wiley & Sons, in over 55 volumes; and “Chemistry of Functional Groups” John Wiley & Sons, in 73 volumes.

Specific and analogous reactants are optionally identified through the indices of known chemicals prepared by the Chemical Abstract Service of the American Chemical Society, which are available in most public and university libraries, as well as through on-line databases (contact the American Chemical Society, Washington, D.C. for more details). Chemicals that are known but not commercially available in catalogs are optionally prepared by custom chemical synthesis houses, where many of the standard chemical supply houses (e.g., those listed above) provide custom synthesis services. A reference useful for the preparation and selection of pharmaceutical salts of the compounds described herein is P. H. Stahl & C. G. Wermuth “Handbook of Pharmaceutical Salts”, Verlag Helvetica Chimica Acta, Zurich, 2002.

### Pharmaceutical Compositions

In certain embodiments, the AKT1 inhibitory compound described herein is administered as a pure chemical. In other embodiments, the AKT1 inhibitory compound described herein is combined with a pharmaceutically suitable or acceptable carrier (also referred to herein as a pharmaceutically suitable (or acceptable) excipient, physiologically suitable (or acceptable) excipient, or physiologically suitable (or acceptable) carrier) selected on the basis of a chosen route of administration and standard pharmaceutical practice as described, for example, in Remington: The Science and Practice of Pharmacy (Gennaro, 21^{st} Ed. Mack Pub. Co., Easton, PA (2005)).

Provided herein is a pharmaceutical composition comprising at least one AKT1 inhibitory compound as described herein, or a stereoisomer, pharmaceutically acceptable salt, hydrate, or solvate thereof, together with one or more pharmaceutically acceptable carriers. The carrier(s) (or excipient(s)) is acceptable or suitable if the carrier is compatible with the other ingredients of the composition and not deleterious to the recipient (i.e., the subject or the patient) of the composition.

One embodiment provides a pharmaceutical composition comprising a pharmaceutically acceptable excipient and a compound of Formula (I), or a pharmaceutically acceptable salt or solvate thereof.

One embodiment provides a method of preparing a pharmaceutical composition comprising mixing a compound of Formula (I), or a pharmaceutically acceptable salt or solvate thereof, and a pharmaceutically acceptable carrier.

In certain embodiments, the AKT1 inhibitory compound as described by Formula (I), or a pharmaceutically acceptable salt or solvate thereof, is substantially pure, in that it contains less than about 5%, or less than about 2%, or less than about 1%, or less than about 0.5%, or less than about 0.1%, of other organic small molecules, such as unreacted intermediates or synthesis by-products that are created, for example, in one or more of the steps of a synthesis method.

One embodiment provides a pharmaceutical composition comprising a pharmaceutically acceptable excipient and a compound of Table 1 or Table 2, or a pharmaceutically acceptable salt or solvate thereof.

One embodiment provides a method of preparing a pharmaceutical composition comprising mixing a compound of Table 1 or Table 2, or a pharmaceutically acceptable salt or solvate thereof, and a pharmaceutically acceptable carrier.

In certain embodiments, the AKT1 inhibitory compound as described by Table 1 or Table 2, or a pharmaceutically acceptable salt or solvate thereof, is substantially pure, in that it contains less than about 5%, or less than about 2%, or less than about 1%, or less than about 0.5%, or less than about 0.1%, of other organic small molecules, such as unreacted intermediates or synthesis by-products that are created, for example, in one or more of the steps of a synthesis method.

Suitable oral dosage forms include, for example, tablets, pills, sachets, or capsules of hard or soft gelatin, methylcellulose or of another suitable material easily dissolved in the digestive tract. In some embodiments, suitable nontoxic solid carriers are used which include, for example, pharmaceutical grades of mannitol, lactose, starch, magnesium stearate, sodium saccharin, talcum, cellulose, glucose, sucrose, magnesium carbonate, and the like. (See, e.g., Remington: The Science and Practice of Pharmacy (Gennaro, 21^{st} Ed. Mack Pub. Co., Easton, PA (2005)).

In some embodiments, the AKT1 inhibitory compound as described by Formula (I) or Table 1 or Table 2, or pharmaceutically acceptable salt or solvate thereof, is formulated for administration by injection. In some instances, the injection formulation is an aqueous formulation. In some instances, the injection formulation is a non-aqueous formulation. In some instances, the injection formulation is an oil-based formulation, such as sesame oil, or the like.

The dose of the composition comprising at least one AKT1 inhibitory compound as described herein differs depending upon the subject or patient's (e.g., human) condition. In some embodiments, such factors include general health status, age, and other factors.

Pharmaceutical compositions are administered in a manner appropriate to the disease to be treated (or prevented). An appropriate dose and a suitable duration and frequency of administration will be determined by such factors as the condition of the patient, the type and severity of the patient's disease, the particular form of the active ingredient, and the method of administration. In general, an appropriate dose and treatment regimen provides the composition(s) in an amount sufficient to provide therapeutic and/or prophylactic benefit (e.g., an improved clinical outcome, such as more frequent complete or partial remissions, or longer disease-free and/or overall survival, or a lessening of symptom severity. Optimal doses are generally determined using experimental models and/or clinical trials. The optimal dose depends upon the body mass, weight, or blood volume of the patient.

Oral doses typically range from about 1.0 mg to about 1000 mg, one to four times, or more, per day.

### Methods of Treatment

One embodiment provides a compound of Formula (I), (Ia), or (1b), or a pharmaceutically acceptable salt or solvate thereof, for use in a method of treatment of the human or animal body.

One embodiment provides a compound of Formula (I), (Ia), or (Ib), or a pharmaceutically acceptable salt or solvate thereof, for use in a method of treatment of cancer or neoplastic disease.

One embodiment provides a pharmaceutical composition comprising a compound of Formula (I), (Ia), or (Ib), or a pharmaceutically acceptable salt or solvate thereof, and a pharmaceutically acceptable excipient for use in a method of treatment of cancer or neoplastic disease.

One embodiment provides a use of a compound of Formula (I), (Ia), or (Ib), or a pharmaceutically acceptable salt or solvate thereof, in the manufacture of a medicament for the treatment of cancer or neoplastic disease.

In some embodiments is provided a method of treating cancer, in a patient in need thereof, comprising administering to the patient a compound of Formula (I), (Ia), or (Ib), or a pharmaceutically acceptable salt or solvate thereof. In some embodiments is provided a method of treating cancer, in a patient in need thereof, comprising administering to the patient a pharmaceutical composition comprising a compound of Formula (I), (Ia), or (Ib), or a pharmaceutically acceptable salt or solvate thereof, and a pharmaceutically acceptable excipient.

One embodiment provides a compound of Table 1 or Table 2, or a pharmaceutically acceptable salt or solvate thereof, for use in a method of treatment of the human or animal body.

One embodiment provides a compound of Table 1 or Table 2, or a pharmaceutically acceptable salt or solvate thereof, for use in a method of treatment of cancer or neoplastic disease.

One embodiment provides a pharmaceutical composition comprising a compound of Table 1 or Table 2, or a pharmaceutically acceptable salt or solvate thereof, and a pharmaceutically acceptable excipient for use in a method of treatment of cancer or neoplastic disease.

One embodiment provides a use of a compound of Table 1 or Table 2, or a pharmaceutically acceptable salt or solvate thereof, in the manufacture of a medicament for the treatment of cancer or neoplastic disease.

In some embodiments is provided a method of treating cancer, in a patient in need thereof, comprising administering to the patient a compound of Table 1 or Table 2, or a pharmaceutically acceptable salt or solvate thereof. In some embodiments is provided a method of treating cancer, in a patient in need thereof, comprising administering to the patient a pharmaceutical composition comprising a compound of Table 1 or Table 2, or a pharmaceutically acceptable salt or solvate thereof, and a pharmaceutically acceptable excipient.

Provided herein is the method wherein the pharmaceutical composition is administered orally. Provided herein is the method wherein the pharmaceutical composition is administered by injection.

One embodiment provides a method of inhibiting a AKT1 enzyme comprising contacting the AKT1 enzyme with a compound of Formula (I), (Ia), or (Ib), or Table 1 or Table 2. Another embodiment provides the method of inhibiting a AKT1 enzyme, wherein the AKT1 enzyme is contacted in an in vivo setting. Another embodiment provides the method of inhibiting a AKT1 enzyme, wherein the AKT1 enzyme is contacted in an in vitro setting.

### Modification of AKT1

Without wishing to be bound by any theory, the AKT1 inhibitory compounds described herein interact with AKT1 E17K by nucleophilic addition of the lysine of residue 17 to the nitrile group of the compounds of Formula (I), (Ia), or (Ib), or Table 1 or Table 2 as indicated in the scheme below.

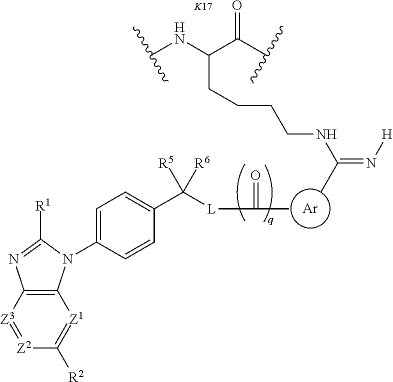

One embodiment provides a modified AKT1 E17K polypeptide wherein the lysine at position 17 of an unmodified AKT1 E17K polypeptide has been modified with a nitrogen substituent having the structure of Formula (X), or a tautomer thereof:

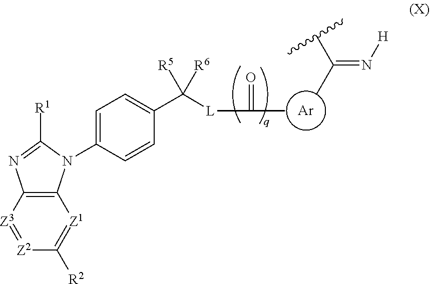

wherein:
   Z¹ is N, C—H, or C—R³;
   Z² is N, C—H, or C—R⁴;
   Z³ is N, C—OH, or C—R⁹;
   Ar is selected from:

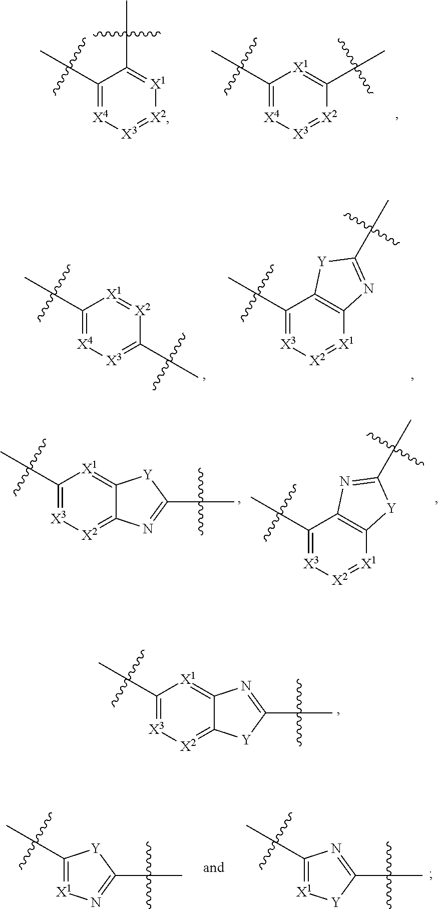

X¹ is N or C—R⁷;
X² is N or C—R⁷;
X³ is N or C—R⁷;
X⁴ is N or C—R⁷;
Y is O, S, or N—R⁹;
R¹ is selected from hydrogen, optionally substituted C1-C6 alkyl, optionally substituted aryl, or optionally substituted heteroaryl;
R² is selected from hydrogen, halogen, —OH, —CN, —N(R⁹)₂, —OR⁹, —SR⁹, —SO₂R⁹, —CO₂R⁹, —CON(R⁹)₂, —SR⁹, —S(O)R⁹, —S(O)₂R⁹, optionally substituted C1-C6 alkyl, optionally substituted C1-C6 alkoxy, optionally substituted C2-C6 alkenyl, optionally substituted C2-C6 alkynyl, optionally substituted C3-C7 carbocyclyl, optionally substituted 4-membered to 6-membered heterocyclyl, optionally substituted aryl, aryl substituted with an optionally substituted 4-membered to 6-membered heterocyclyl, optionally substituted heteroaryl or heteroaryl substituted with an optionally substituted 3-membered to 6-membered carbocyclyl;
R³ is selected from optionally substituted C1-C6 alkyl, or optionally substituted aryl;
R⁴ is selected from halogen, —CN, optionally substituted C1-C6 alkyl, or optionally substituted aryl;
R⁵ and R⁶ are each independently hydrogen, deuterium, halogen, —OH, or optionally substituted C1-C6 alkyl; or R⁵ and R⁶ together form an oxo; or R⁵ and R⁶ join together to form a carbocycle or heterocycle;
each R⁷ is independently selected from hydrogen, deuterium, halogen, —OH, —SH, optionally substituted C1-C6 alkoxy, —S— (optionally substituted C1-C6 alkyl), —CN, optionally substituted C1-C6 alkyl, and optionally substituted aryl;
L is selected from —N(R⁸)—, or a divalent radical selected from:

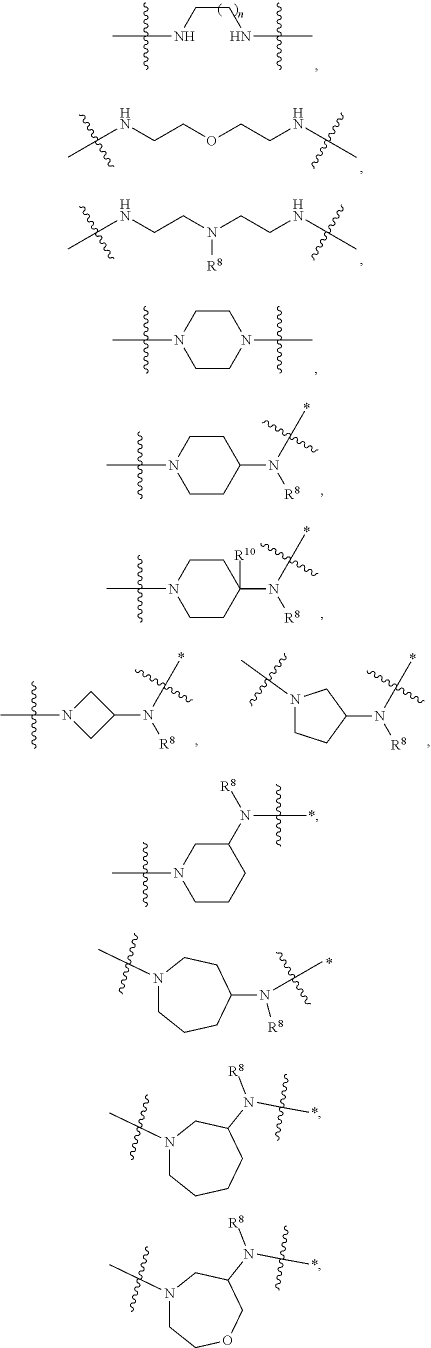

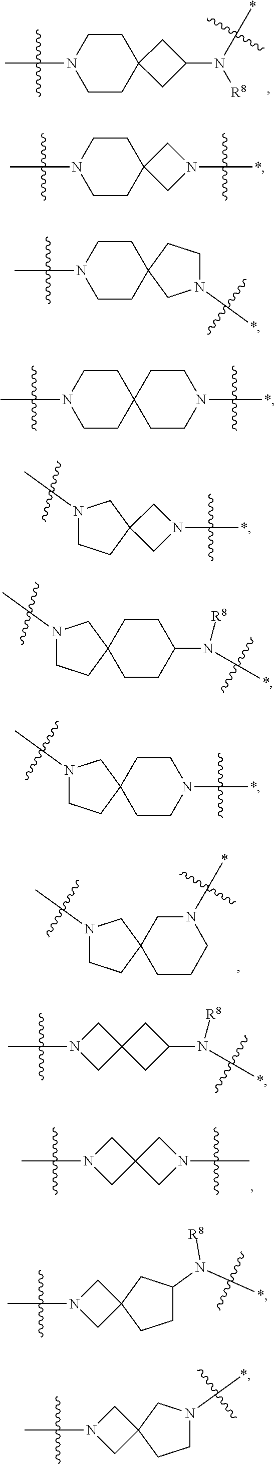

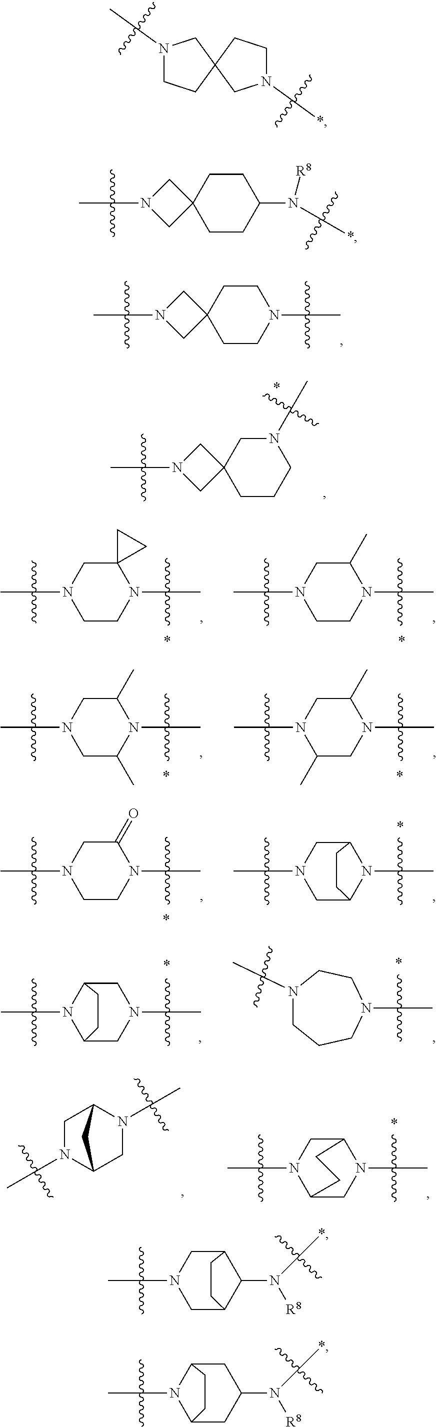

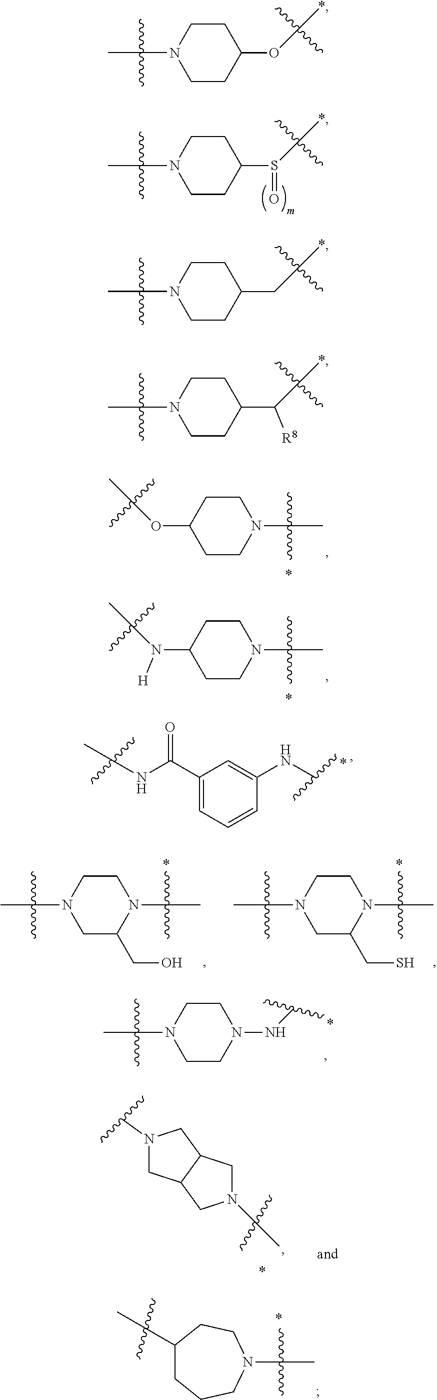

wherein the asterisk (*) indicates the bond to the —CO—Ar group;
   R⁸ is hydrogen, optionally substituted C1-C6 alkyl, optionally substituted C3-C7 cycloalkyl, or optionally substituted heterocyclyl;
   each R⁹ is hydrogen, or optionally substituted C1-C6 alkyl; R¹⁰ is optionally substituted C1-C6 alkyl;
   m is 0, 1, or 2;
   n is 1, 2, or 3; and
   q is 0 or 1.

Another embodiment provides the modified AKT1 E17K polypeptide of Formula (X), wherein Z¹ is N.

Another embodiment provides the modified AKT1 E17K polypeptide of Formula (X), wherein Z² is C—H.

Another embodiment provides the modified AKT1 E17K polypeptide of Formula (X), wherein Z² is C—R⁴.

Another embodiment provides the modified AKT1 E17K polypeptide of Formula (X), wherein R¹ is optionally substituted heteroaryl. Another embodiment provides the modified AKT1 E17K polypeptide of Formula (X), wherein the optionally substituted heteroaryl is an optionally substituted pyridyl.

Another embodiment provides the modified AKT1 E17K polypeptide of Formula (X), wherein R² is optionally substituted aryl. Another embodiment provides the modified AKT1 E17K polypeptide of Formula (X), wherein the optionally substituted aryl is an optionally substituted phenyl.

Another embodiment provides the modified AKT1 E17K polypeptide of Formula (X), wherein R² is optionally substituted heteroaryl. Another embodiment provides the modified AKT1 E17K polypeptide of Formula (X), wherein the optionally substituted heteroaryl is an optionally substituted pyridyl.

Another embodiment provides the modified AKT1 E17K polypeptide of Formula (X), wherein R⁵ is hydrogen. Another embodiment provides the modified AKT1 EI7K polypeptide of Formula (X), wherein R⁶ is hydrogen. Another embodiment provides the modified AKT1 E17K polypeptide of Formula (X), wherein Rand R⁶ together form an oxo. Another embodiment provides the modified AKT1 E17K polypeptide of Formula (X), wherein R⁶ is optionally substituted C1-C6 alkyl. Another embodiment provides the modified AKT1 E17K polypeptide of Formula (X), wherein R and R⁶ join together to form a carbocycle or heterocycle.

Another embodiment provides the modified AKT1 E17K polypeptide of Formula (X), wherein L is —N(R^{a})—.

Another embodiment provides the modified AKT1 E17K polypeptide of Formula (X), wherein L is selected from

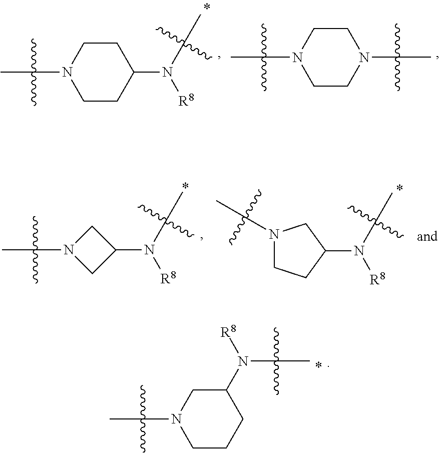

Another embodiment provides the modified AKT1 E17K polypeptide of Formula (X), wherein L is selected from:

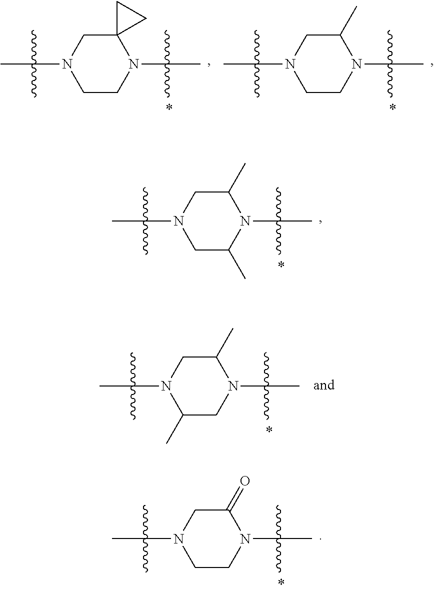

Another embodiment provides the modified AKT1 E17K polypeptide of Formula (X), wherein L is selected from:

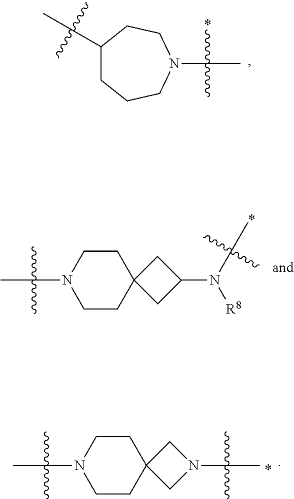

Another embodiment provides the modified AKT1 E17K polypeptide of Formula (X), wherein L is selected from:

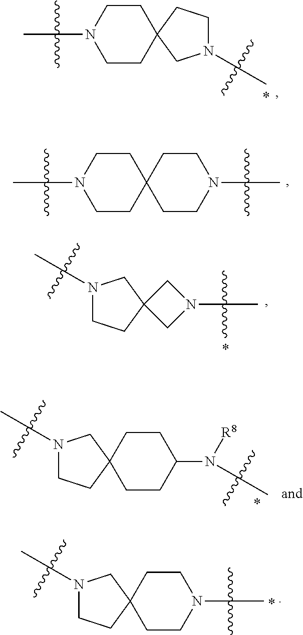

Another embodiment provides the modified AKT1 E17K polypeptide of Formula (X), wherein L is selected from:

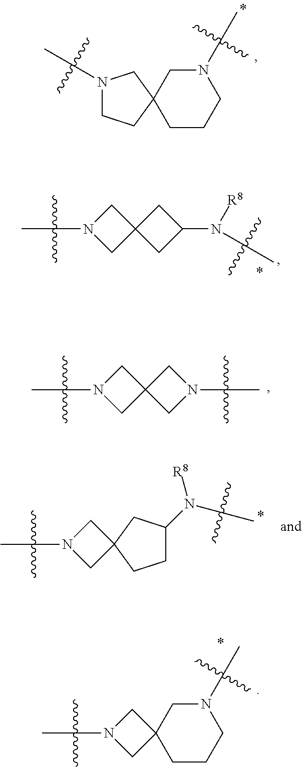

Another embodiment provides the modified AKT1 E17K polypeptide of Formula (X), wherein L is selected from:

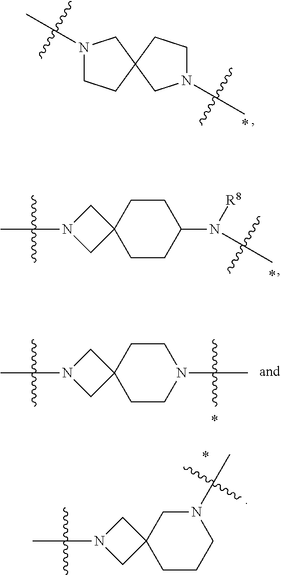

Another embodiment provides the modified AKT1 E17K polypeptide of Formula (X), wherein R⁸ is hydrogen or optionally substituted C1-C6 alkyl.

Another embodiment provides the modified AKT1 E17K polypeptide of Formula (X), wherein q is 0.

Another embodiment provides the modified AKT1 E17K polypeptide of Formula (X), wherein q is 1.

Another embodiment provides the modified AKT1 E17K polypeptide of Formula (X), wherein Ar is

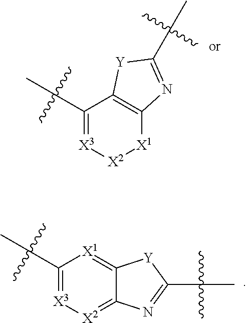

Another embodiment provides the modified AKT1 E17K polypeptide of Formula (X), wherein Ar is

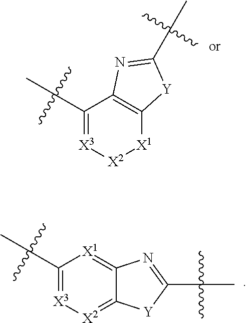

Another embodiment provides the modified AKT1 E17K polypeptide of Formula (X), wherein Ar is

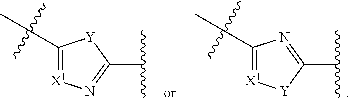

Another embodiment provides the modified AKT1 E17K polypeptide of Formula (X), wherein X¹, X², and X³ are C—H. Another embodiment provides the modified AKT1 E17K polypeptide of Formula (X), wherein X¹ is N; and X², and X³ are C—H. Another embodiment provides the modified AKT1 E17K polypeptide of Formula (X), wherein X² is N; and X¹, and X³ are C—H.

Another embodiment provides the modified AKT1 E17K polypeptide of Formula (X), wherein X³ is N, and X¹, and X² are C—H. Another embodiment provides the modified AKT1 E17K polypeptide of Formula (X), wherein X¹ is N; and X², and X³ are C—H.

Another embodiment provides the modified AKT1 E17K polypeptide of Formula (X), wherein Y is O. Another embodiment provides the modified AKT1 E17K polypeptide of Formula (X), wherein Y is S. Another embodiment provides the modified AKT1 E17K polypeptide of Formula (X), wherein Y is N—R⁹. Another embodiment provides the modified AKT1 E17K polypeptide of Formula (X), wherein R⁹ is hydrogen. Another embodiment provides the modified AKT1 E17K polypeptide of Formula (X), wherein R⁹ is optionally substituted C1-C6 alkyl.

Another embodiment provides the modified AKT1 E17K polypeptide of Formula (X), wherein Ar is

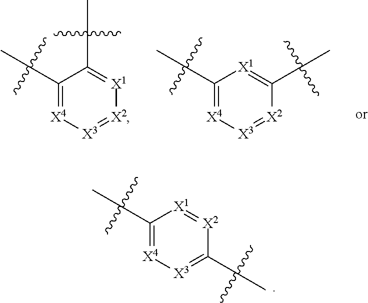

Another embodiment provides the modified AKT1 E17K polypeptide of Formula (X), wherein X¹ is N. Another embodiment provides the modified AKT1 E17K polypeptide of Formula (X), wherein X² is N. Another embodiment provides the modified AKT1 E17K polypeptide of Formula (X), wherein X³ is N. Another embodiment provides the modified AKT1 E17K polypeptide of Formula (X), wherein X⁴ is N. Another embodiment provides the modified AKT1 E17K polypeptide of Formula (X), X¹ is C—H. Another embodiment provides the modified AKT1 E17K polypeptide of Formula (X), X² is C—H. Another embodiment provides the modified AKT1 E17K polypeptide of Formula (X), X³ is C—H. Another embodiment provides the modified AKT1 E17K polypeptide of Formula (X), wherein X⁴ is C—H.

Another embodiment provides the modified AKT1 E17K polypeptide of Formula (X), wherein R³ is optionally substituted C1-C6 alkyl. Another embodiment provides the modified AKT1 E17K polypeptide of Formula (X), wherein R³ is optionally substituted aryl. Another embodiment provides the modified AKT1 E17K polypeptide of Formula (X), wherein R⁴ is optionally substituted C1-C6 alkyl. Another embodiment provides the modified AKT1 E17K polypeptide of Formula (X), wherein R⁴ is optionally substituted aryl.

Another embodiment provides the modified AKT1 E17K polypeptide of Formula (X), wherein Ar is

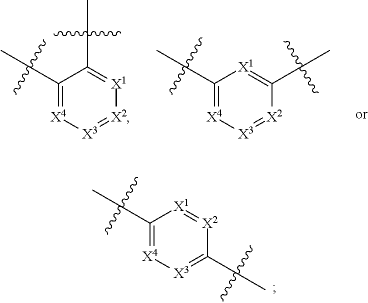

and L is selected from:

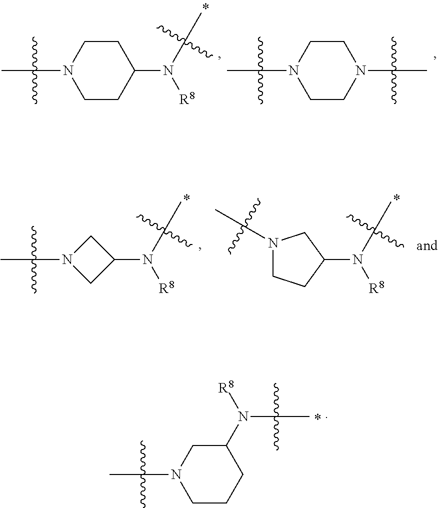

Another embodiment provides the modified AKT1 E17K polypeptide of Formula (X), wherein Ar is

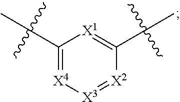

### and L is

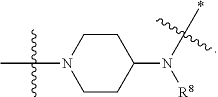

Another embodiment provides the modified AKT1 E17K polypeptide of Formula (X), wherein Ar is

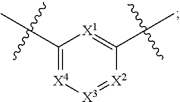

### and L is

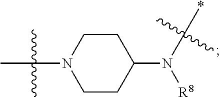

and q is 0.

Another embodiment provides the modified AKT1 E17K polypeptide of Formula (X), wherein Ar is

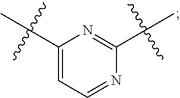 [SMILES: *c1ccnc(*)n1]

and L is selected from:

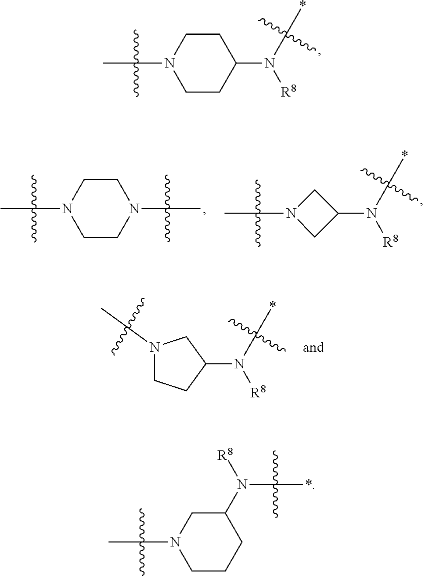

Another embodiment provides the modified AKT1 E17K polypeptide of Formula (X), wherein Ar is

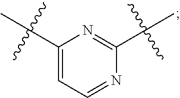 [SMILES: *c1ccnc(*)n1]

and R¹ is optionally substituted heteroaryl, and R² is optionally substituted heteroaryl.

Another embodiment provides the modified AKT1 E17K polypeptide wherein the unmodified AKT1 E17K polypeptide is a SEQID selected from a SEQID provided in Table 3.

TABLE 3

| SEQ | |
|---|---|
| ID | |
| NO | Amino Acid Sequence |
| 1 | MSDVAIVKEGWLHKRGKYIKTWRPRYFLLKNDGTFIGYKERPQDVD |
| | QREAPLNNFSVAQCQLMKTERPRPNTFIIRCLQWTTVIERTFHVET |
| | PEEREEWTTAIQTVADGLKKQEEEEMDFRSGSPSDNSGAEEMEVSL |
| | AKPKHRVTMNEFEYLKLLGKGTFGKVILVKEKATGRYYAMKILKKE |
| | VIVAKDEVAHTLTENRVLQNSRHPFLTALKYSFQTHDRLCFVMEYA |
| | NGGELFFHLSRERVFSEDRARFYGAEIVSALDYLHSEKNVVYRDLK |
| | LENLMLDKDGHIKITDEGLCKEGIKDGATMKTFCGTPEYLAPEVLE |
| | DNDYGRAVDWWGLGVVMYEMMCGRLPFYNQDHEKLFELILMEEIRF |
| | PRTLGPEAKSLLSGLLKKDPKQRLGGGSEDAKEIMQHRFFAGIVWQ |
| | HVYEKKLSPPFKPQVTSETDTRYFDEEFTAQMITITPPDQDDSMEC |
| | VDSERRPHFPQFSYSASGTA |

Other embodiments and uses will be apparent to one skilled in the art in light of the present disclosures. The following examples are provided merely as illustrative of various embodiments and shall not be construed to limit the invention in any way.

### EXAMPLES

### I. Chemical Synthesis

In some embodiments, the AKT1 inhibitory compounds disclosed herein are synthesized according to the following examples. As used below, and throughout the description of the invention, the following abbreviations, unless otherwise indicated, shall be understood to have the following meanings:
ACN acetonitrile
° C. degrees Celsius
δH chemical shift in parts per million downfield from tetramethylsilane
DCM dichloromethane (CH₂Cl₂)
DIAD diisopropyl azodicarboxylate
DIEA diisopropylethylamine
DMF dimethylformamide
DMSO dimethylsulfoxide
EA ethyl acetate
EtOAc ethyl acetate
ESI electrospray ionization
Et ethyl
g gram(s)
h hour(s)
HPLC high performance liquid chromatography
Hz hertz
J coupling constant (in NMR spectrometry)
LCMS liquid chromatography mass spectrometry
μ micro
m multiplet (spectral); meter(s); milli
M molar
M⁺ parent molecular ion
Me methyl
MsCl methanesulfonyl chloride
MHz megahertz
min minute(s)
mol mole(s); molecular (as in mol wt)
mL milliliter
MS mass spectrometry
nm nanometer(s)
NMR nuclear magnetic resonance
pH potential of hydrogen; a measure of the acidity or basicity of an aqueous solution
PE petroleum ether
RT room temperature
s singlet (spectral)
t triplet (spectral)
SFC Supercritical fluid chromatography
T temperature
TFA trifluoroacetic acid
THF tetrahydrofuran
TPP Triphenylphosphine

### Experimental Procedures

### Intermediate 1: 3-(3-(4-(Aminomethyl)phenyl)-3H-imidazo[4,5-b]pyridin-2-yl)pyridin-2-amine

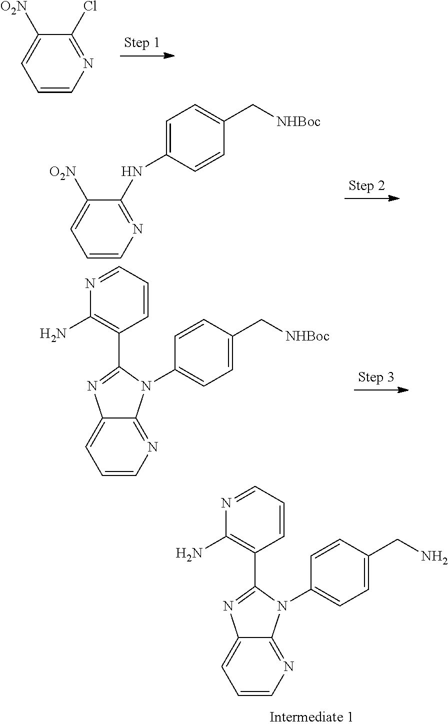

### Step 1: Tert-butyl 4-((3-nitropyridin-2-yl)amino)benzylcarbamate

To a solution of 2-chloro-3-nitro-pyridine (7.0 g, 44.2 mmol) and tert-butyl N-[(4-aminophenyl)methyl]carbamate (9.8 g, 44.2 mmol) in DMSO (100 mL) was added DIEA (11.4 g, 88.3 mmol). The mixture was stirred at 80° C. for 12 hr. The reaction mixture was diluted with 100 mL H₂O at 25° C. and extracted with EtOAc (100 mL×3). The combined organic layers were washed with brine (150 mL), dried over Na₂SO₄, filtered, and concentrated under reduced pressure to give a residue. The residue was purified by triturated (petroleum ether:EtOAc=10:1) to give tert-butyl N-[[4-[(3-nitro-2-pyridyl)amino]phenyl]methyl]carbamate (13.9 g, yield: 91%) as a red solid. MS: m/z=344.8 [M+H]⁺. ¹H NMR (400 MHz, Dimethylsulfoxide-d₆) δ 9.90 (s, 1H), 8.49 (dd, J=8.4, 1.6 Hz, 1H), 8.45 (dd, J=4.4, 1.6 Hz, 1H), 7.52 (d, J=8.4 Hz, 2H), 7.35 (t, J=6.0 Hz, 1H), 7.19 (d, J=8.4 Hz, 2H), 6.93 (dd, J=8.4, 4.4 Hz, 1H), 4.07 (d, J=6.0 Hz, 2H), 1.36 (s, 9H).

### Step 2: Tert-butyl 4-(2-(2-aminopyridin-3-yl)-3H-imidazo[4,5-b]pyridin-3-yl)benzylcarbamate

To a solution of tert-butyl N-[[4-[(3-nitro-2-pyridyl)amino]phenyl]methyl]carbamate (10 g, 29.0 mmol) in MeOH (70 mL) and DMSO (140 mL) was added 2-aminopyridine-3-carbaldehyde (3.9 g, 31.9 mmol) and Na₂S₂O₄ (10.1 g, 58.1 mmol). The mixture was stirred at 100° C. for 12 hr. After cooling to room temperature, the reaction mixture was diluted with 200 mL of H₂O, and extracted with EtOAc (400 mL×2). The combined organic layers were washed with brine (400 mL), dried over Na₂SO₄, filtered, and concentrated under reduced pressure to give a residue. The crude product was purified by flash chromatography on silica gel (Eluent of 1˜2% MeOH in CH₂Cl₂) to give tert-butyl 4-(2-(2-aminopyridin-3-yl)-3H-imidazo[4,5-b]pyridin-3-yl)benzylcarbamate (5.7 g, yield: 44%) as a red solid. MS: m/z=417.4 [M+H]⁺. ¹H NMR (400 MHz, Dimethylsulfoxide-d₆) δ 8.31 (dd, J=4.8, 1.2 Hz, 1H), 8.20 (dd, J=8.0, 1.2 Hz, 1H), 7.99 (dd, J=4.8, 1.6 Hz, 1H), 7.49 (t, J=6.0 Hz, 1H), 7.41-7.36 (m, 5H), 7.21 (dd, J=7.6, 1.6 Hz, 1H), 6.99 (br s, 2H), 6.39 (dd, J=7.6, 4.8 Hz, 1H), 4.21 (d, J=6.0 Hz, 2H), 1.41 (s, 9H).

### Step 3: 3-(3-(4-(Aminomethyl)phenyl)-3H-imidazo[4,5-b]pyridin-2-yl)pyridin-2-amine

To a solution of tert-butyl N-[[4-[2-(2-amino-3-pyridyl)imidazo[4,5-b]pyridin-3-yl]phenyl]methyl]carbamate (350 mg, 840 μmol) in 1,4-dioxane (3 mL) was added 4 M HCl in 1,4-dioxane (I mL) at 20° C. The mixture was stirred at 20° C. for 2 hr. The reaction was concentrated under reduced pressure to give crude product 280 mg (HCl salt, yield: 95%). The crude product was purified by prep-HPLC (Column: Phenomenex luna C18 150×25 mm×10 μm; Condition: water (HCl)-ACN; Begin B: 0; End B: 16; Gradient Time (min): 10; 100% B Hold Time (min): 2; Flow Rate (mL/min): 25) to give product (HCl salt). The product was diluted with 10 mL aqueous NaHCO₃ and extracted with DCM (10 mL×3). The combined organic layers were washed with 20 mL brine, dried over Na₂SO₄, filtered and concentrated under reduced pressure to give 3-(3-(4-(Aminomethyl)phenyl)-3H-imidazo[4,5-b]pyridin-2-yl)pyridin-2-amine (Intermediate 1, 70.0 mg, yield: 95%) was obtained as a light-yellow solid. MS: m/z=317.1 [M+H]⁺. ¹H NMR (400 MHz, Dimethylsulfoxide-d₆) δ 8.31 (dd, J=4.8, 1.2 Hz, 1H), 8.19 (dd, J=8.0, 1.2 Hz, 1H), 7.99 (dd, J=4.8, 1.6 Hz, 1H), 7.47 (d, J=8.4 Hz, 2H), 7.40-7.33 (m, 3H), 7.22 (dd, J=7.6, 2.0 Hz, 1H), 6.98 (br s, 2H), 6.40 (dd, J=7.6, 4.8 Hz, 1H), 3.79 (s, 2H), 1.82 (br s, 2H).

### Intermediate 2: 3-(3-(4-(Piperazin-1-ylmethyl)phenyl)-3H-imidazo[4,5-b]pyridin-2-yl)pyridin-2-amine

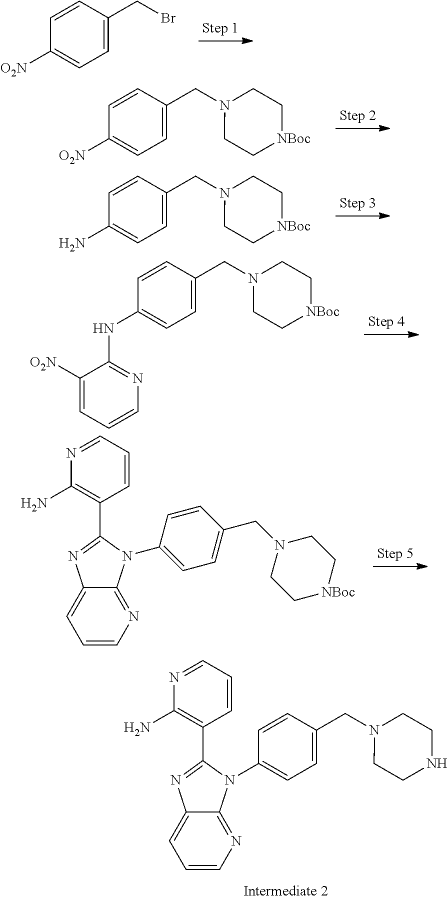

### Step 1: Tert-butyl 4-(4-nitrobenzyl)piperazine-1-carboxylate

To a solution of 1-(bromomethyl)-4-nitro-benzene (25 g, 116 mmol) in ACN (250 mL) was added tert-butyl piperazine-1-carboxylate (25.8 g, 139 mmol) and K₂CO₃ (31.9 g, 231 mmol) at 20° C. The mixture was stirred at 20° C. for 12 hr. The reaction mixture was filtered. The filter liquor was concentrated to dryness to give a crude tert-butyl 4-(4-nitrobenzyl)piperazine-1-carboxylate (37 g, yield: 99%) as a white solid, which was used in the next step without further purification. MS: m/z=322.2 [M+H]⁺. ¹H NMR (400 MHz, Dimethylsulfoxide-d₆) δ 8.19 (d, J=8.8 Hz, 2H), 7.60 (d, J=8.8 Hz, 2H), 3.62 (s, 2H), 3.32-3.36 (m, 4H), 2.38-2.28 (m, 4H), 1.39 (s, 9H).

### Step 2: Tert-butyl 4-(4-aminobenzyl)piperazine-1-carboxylate

To a solution of tert-butyl 4-(4-nitrobenzyl)piperazine-1-carboxylate (20 g, 62.2 mmol) in EtOH (150 mL) and H₂O (50 mL) was added Fe (17.3 g, 311 mmol) and NH₄Cl (13.3 g, 249 mmol) at 25° C., the mixture was stirred at 90° C. for 2 hr. The reaction mixture was filtered and concentrated directly to give a crude product tert-butyl 4-(4-aminobenzyl)piperazine-1-carboxylate (17 g, crude) as a yellow oil, which was used in the next step without further purification. MS: m/z=292.9 [M+H]⁺.

### Step 3: Tert-butyl 4-(4-((3-nitropyridin-2-yl)amino)benzyl)piperazine-1-carboxylate

To a solution of 2-chloro-3-nitro-pyridine (10 g, 63 mmol) in DMSO (200 mL) was added tert-butyl 4-(4-aminobenzyl)piperazine-1-carboxylate (15.3 g, 52.5 mmol) and DIEA (13.5 g, 105 mmol) at 25° C. The mixture was stirred at 80° C. for 12 hr. The reaction mixture was concentrated to give a residue. The residue was diluted with H₂O (200 mL) and extracted with CH₂Cl₂ (200 mL×2). The combined organic layers were washed with brine (200 mL), dried over Na₂SO₄, filtered, and concentrated under reduced pressure to give a residue. The residue was purified by flash chromatography on silica gel (Eluent of 10˜30% EtOAc in petroleum ether) to give tert-butyl 4-(4-((3-nitropyridin-2-yl)amino)benzyl)piperazine-1-carboxylate (11 g, yield: 45%) as a red solid. MS: m/z=413.9 [M+H]⁺. ¹H NMR (400 MHz, Dimethylsulfoxide-d₆) δ 9.95 (s, 1H), 8.48-8.58 (m, 2H), 7.60 (d, J=8.4 Hz, 2H), 7.29 (d, J=8.4 Hz, 2H), 6.98 (dd, J=8.4, 4.8 Hz, 1H), 3.46 (s, 2H), 3.29-3.32 (m, 4H), 2.29-2.35 (m, 4H), 1.39 (s, 9H).

### Step 4: Tert-butyl 4-(4-(2-(2-Aminopyridin-3-yl)-3H-imidazo[4,5-b]pyridin-3-yl)benzyl)piperazine-1-carboxylate

To a solution of tert-butyl 4-(4-((3-nitropyridin-2-yl)amino)benzyl)piperazine-1-carboxylate (10 g, 24.1 mmol) in DMSO (200 mL), added 2-aminopyridine-3-carbaldehyde (3.54 g, 29.0 mmol) and Na₂S₂O₄ (12.6 g, 72.5 mmol) at 25° C. The mixture was stirred at 100° C. for 14 hr. The reaction mixture was poured into 500 mL of H₂O. The mixture was extracted with CH₂Cl₂ (200 mL×2). The combined organic layers were washed with brine (400 mL), dried over anhydrous Na₂SO₄, filtered, and concentrated to dryness. The residue was purified by flash chromatography on silica gel (Eluent of 10˜30% MeOH in CH₂Cl₂) to give 4-(4-(2-(2-Aminopyridin-3-yl)-3H-imidazo[4,5-b]pyridin-3-yl)benzyl)piperazine-1-carboxyla (4.9 g, yield: 42%) as a red solid. MS: m/z=486.2 [M+H]⁺. ¹H NMR (400 MHz, Dimethylsulfoxide-d₆) δ 8.33 (dd, J=4.8, 1.2 Hz, 1H), 8.20 (dd, J=8.0, 1.2 Hz, 1H), 7.99 (dd, J=4.8, 2.0 Hz, 1H), 7.43-7.48 (m, 2H), 7.36-7.42 (m, 3H), 7.16 (dd, J=7.6, 1.6 Hz, 1H), 7.00 (br s, 2H), 6.38 (dd, J=7.6, 4.8 Hz, 1H), 3.56 (s, 2H), 3.33-3.37 (m, 4H), 2.32-2.38 (m, 4H), 1.40 (s, 9H).

### Step 5: 3-(3-(4-(Piperazin-1-ylmethyl)phenyl)-3H-imidazo[4,5-b]pyridin-2-yl)pyridin-2-amine

To a solution of tert-butyl 4-(4-(2-(2-aminopyridin-3-yl)-3H-imidazo[4,5-b]pyridin-3-yl)benzyl)piperazine-1-carboxylate (2 g, 4.12 mmol) in CH₂Cl₂ (10 mL) was added dropwise TFA (4.62 g, 40.5 mmol) at 25° C., the mixture was stirred at 25° C. for 3 hr. The reaction mixture was concentrated to a residue. The residue was poured into water (50 mL), then adjusted pH to about 8 by saturated NaHCO₃(aq). The resulting mixture was extracted with CH₂Cl₂ (100 mL×3). The combined organic layers were dried over anhydrous Na₂SO₄, filtered, and concentrated to give 3-(3-(4-(Piperazin-1-ylmethyl)phenyl)-3H-imidazo[4,5-b]pyridin-2-yl)pyridin-2-amine (1.43 g, yield: 90%) as an off-white solid. 100 mg of solid was triturated with EtOAc (3 mL) at 25° C. for 1 hr and filtered. The filter cake was collected to give 3-(3-(4-(Piperazin-1-ylmethyl)phenyl)-3H-imidazo[4,5-b]pyridin-2-yl)pyridin-2-amine (Intermediate 2, 24.5 mg, yield: 90%). MS: m/z=386.2 [M+H]⁺. ¹H NMR (400 MHz, Dimethylsulfoxide-d₆) δ 8.33 (d, J=4.0 Hz, 1H), 8.22 (d, J=8.0 Hz, 1H), 8.00 (d, J=3.2 Hz, 1H), 7.35-7.52 (m, 5H), 7.18 (d, J=7.2 Hz, 1H), 7.00 (br s, 2H), 6.40 (dd, d, J=7.8, 4.8 Hz, 1H), 3.63 (s, 2H), 3.16-3.01 (m, 4H), 2.63-2.48 (ms, 4H).

### Intermediate 3: 3-(3-(4-((4-aminopiperidin-1-yl)methyl)phenyl)-3H-imidazo[4,5-b]pyridin-2-yl)pyridin-2-amine

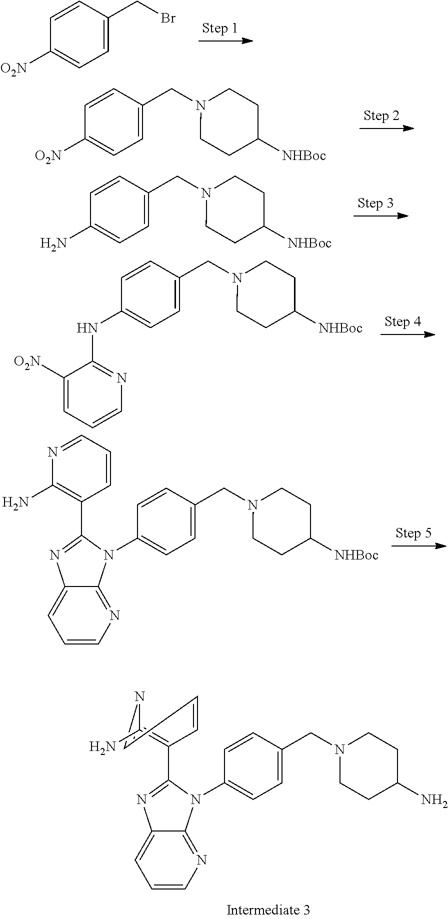

### Step 1: Tert-butyl (1-(4-nitrobenzyl) piperidin-4-yl) carbamate

To a solution of 1-(bromomethyl)-4-nitro-benzene (108 g, 499 mmol) in ACN (1.5 L) was added K₂CO₃ (149 g, 1.1 mol) and tert-butyl N-(4-piperidyl)carbamate (100 g, 499 mmol). The mixture was stirred at 25° C. for 16 hr. The reaction mixture was filtered. The filtrate was concentrated under reduced pressure to give tert-butyl (1-(4-nitrobenzyl) piperidin-4-yl)carbamate (167 g, crude) as a yellow solid, which was used to the next step without further purification. MS: m/z=335.9 [M+H]⁺. ¹H NMR (400 MHz, Chloroform-d) δ 8.16 (d, J=8.8 Hz, 2H), 7.49 (d, J=8.8 Hz, 2H), 4.44 (br s, 1H), 3.56 (s, 2H), 3.52-3.42 (m, 1H), 2.77-2.74 (m, 2H), 2.16-2.10 (m, 2H), 1.93-1.90 (m, 2H), 1.43 (s, 9H), 1.42-1.36 (m, 2H).

### Step 2: Tert-butyl (1-(4-aminobenzyl) piperidin-4-yl) carbamate

To a solution of tert-butyl (1-(4-nitrobenzyl) piperidin-4-yl)carbamate (109 g, 325 mmol) in EtOH (500 mL) and H₂O (150 mL) was added Fe (91 g, 1.6 mol) and NH₄Cl (174 g, 3.3 mol). The mixture was stirred at 85° C. for 2 hr. The reaction mixture was filtered. The filtrate was concentrated under pressure to remove most of the EtOH. The residue was diluted with 500 mL of H₂O and extracted with CH₂Cl₂ (500 mL×2). The combined organic layers were washed with 500 mL of brine, dried over Na₂SO₄, filtered, and concentrated under reduced pressure to give tert-butyl (1-(4-aminobenzyl) piperidin-4-yl) carbamate (80 g crude) as a yellow solid. MS: m/z=306.2 [M+H]+. ¹H NMR (400 MHz, Chloroform-d) δ 7.14 (d, J=8.0 Hz, 2H), 6.64 (d, J=8.0 Hz, 2H), 4.51 (br d, J=6.0 Hz, 1H), 3.80-3.59 (m, 2H), 3.55 (s, 2H), 3.51-3.39 (m, 1H), 2.95-2.93 (m, 2H), 2.25-2.20 (m, 2H), 1.96-1.93 (m, 2H), 1.72-1.54 (m, 2H), 1.42 (s, 9H).

### Step 3: Tert-butyl (1-(4-((3-nitropyridin-2-yl) amino)benzyl)piperidin-4-yl)carbamate

To a solution of tert-butyl (1-(4-aminobenzyl) piperidin-4-yl) carbamate (30 g, 98.2 mmol) in DMSO (500 mL) was added DIEA (38.1 g, 295 mmol) and 2-chloro-3-nitro-pyridine (18.7 g, 118 mmol). The mixture was stirred at 100° C. for 16 hr. The reaction mixture was quenched with H₂O (500 mL) at 20° C. and extracted with EtOAc (300 mL×2). The combined organic layers were washed with 300 mL of brine, dried over Na₂SO₄, filtered, and concentrated under reduced pressure to give a residue. The crude product was purified by flash chromatography on silica gel (Eluent of 0˜10% MeOH in CH₂Cl₂) to give tert-butyl (1-(4-((3-nitropyridin-2-yl) amino) benzyl) piperidin-4-yl) carbamate (30 g, yield: 71%) as a yellow solid. MS: m/z=428.2 [M+H]⁺. ¹H NMR (400 MHz, Chloroform-d) δ 10.10 (s, 1H), 8.51 (dd, J=8.0, 1.6 Hz, 1H), 8.47 (dd, J=8.4, 1.6 Hz, 1H), 7.58 (d, J=8.4 Hz, 2H), 7.32 (d, J=8.4 Hz, 2H), 6.81 (dd, J=8.4, 4.4 Hz, 1H), 4.44 (br s, 1H), 3.47 (s, 2H), 3.44-3.34 (m, 1H), 2.82-2.79 (m, 2H), 2.13-2.05 (m, 2H), 1.93-1.89 (m, 2H), 1.43 (s, 9H), 1.39-1.37 (m, 2H).

### Step 4: Tert-butyl (1-(4-(2-(2-aminopyridin-3-yl)-3H-imidazo[4,5-b]pyridin-3-yl)benzyl)piperidin-4-yl)carbamate

To a solution of (12.5 g, 29.2 mmol) in DMSO (500 mL) were added Na₂S₂O₄ (15.3 g, 87.7 mmol) and 2-aminopyridine-3-carbaldehyde (4.3 g, 35.1 mmol). The mixture was stirred at 100° C. for 16 hr. The reaction mixture was quenched with H₂O (1000 mL) at 20° C. and extracted with EtOAc (1000 mL×2). The combined organic layers were washed with brine (500 mL), dried over Na₂SO₄, filtered, and concentrated under reduced pressure to give a residue. The crude product was purified by flash chromatography on silica gel (Eluent of 0˜10% MeOH in CH₂Cl₂) to give tert-butyl (1-(4-(2-(2-aminopyridin-3-yl)-3H-imidazo[4,5-b]pyridin-3-yl)benzyl)piperidin-4-yl)carbamate (5.5 g, yield: 38%) as a yellow solid. MS: m/z=500.2 [M+H]+. ¹H NMR (400 MHz, Chloroform-d) δ 8.46-8.37 (m, 1H), 8.12-8.02 (m, 2H), 7.48 (d, J=8.0 Hz, 2H), 7.34-7.28 (m, 3H), 7.07 (dd, J=8.0, 4.0 Hz, 1H), 6.62 (br s, 2H), 6.33 (dd, J=7.6, 4.8 Hz, 1H), 4.46 (br d, J=6.0 Hz, 1H), 3.56 (s, 2H), 3.49-3.47 (m, 1H), 2.84 (br d, J=11.2 Hz, 2H), 2.14 (t, J=12.0 Hz, 2H), 1.93 (br d, J=11.2 Hz, 2H), 1.45 (s, 9H), 1.51-1.38 (m, 2H).

### Step 5. 3-(3-(4-((4-Aminopiperidin-1-yl)methyl)phenyl)-3H-imidazo[4,5-b]pyridin-2-yl)pyridin-2-amine

A solution of tert-butyl (1-(4-(2-(2-aminopyridin-3-yl)-3H-imidazo[4,5-b]pyridin-3-yl)benzyl)piperidin-4-yl)carbamate (2.0 g, 4.0 mmol) in HCl/1,4-dioxane (4 M, 20 mL) was stirred at 25° C. for 1 hr. The reaction mixture was concentrated under reduced pressure to remove solvent. Then residue was quenched with NaHCO₃ (30 mL) at 20° C., then MeOH was added, filtered, and the filtrate after freeze-dried to give 3-(3-(4-((4-aminopiperidin-1-yl)methyl)phenyl)-3H-imidazo[4,5-b]pyridin-2-yl)pyridin-2-amine (Intermediate 3, 1.45 g, yield: 91%) as a yellow solid. MS: m/z=400.1 [M+H]⁺. ¹H NMR (400 MHz, Dimethylsulfoxide-d₆) δ 8.37-8.28 (m, 1H), 8.19 (dd, J=8.0, 4.0 Hz, 1H), 7.99-7.97 (m, 1H), 7.47-7.32 (m, 5H), 7.15 (dd, J=7.6, 1.6 Hz, 1H), 7.01 (br s, 2H), 6.36 (dd, J=6.8, 4.0 Hz, 1H), 3.50 (s, 2H), 3.27-3.23 (m, 1H), 2.75 (br d, J=10.8 Hz, 2H), 2.00 (t, 0.1=10.8 Hz, 2H), 1.72 (d, J=10.8 Hz, 2H), 1.39-1.31 (m, 2H).

### Intermediate 4: 3-(3-(4-(Aminomethyl)phenyl)-5-phenyl-3H-imidazo[4,5-b]pyridin-2-yl)pyridin-2-amine

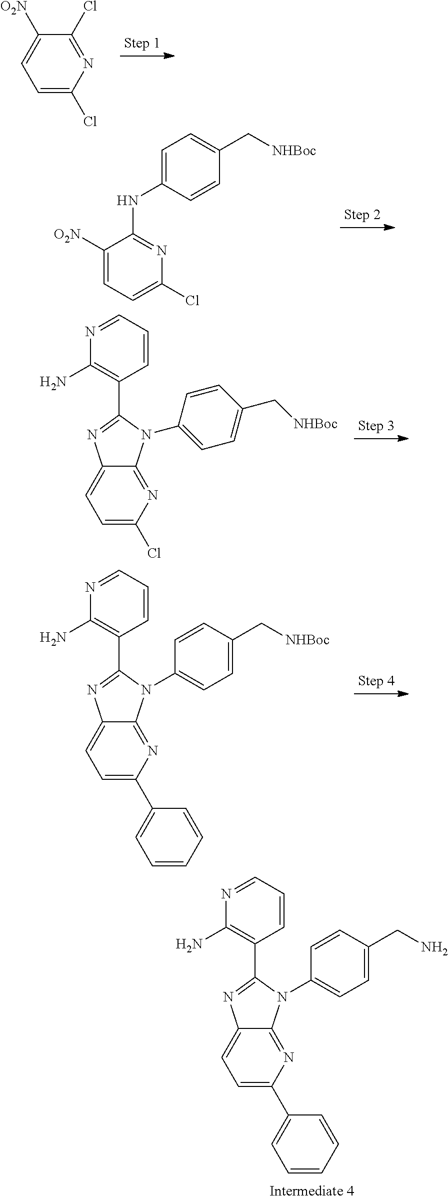

### Step 1: Tert-butyl 4-((6-chloro-3-nitropyridin-2-yl)amino)benzylcarbamate

To a solution of 2,6-dichloro-3-nitro-pyridine (2.0 g, 10.4 mmol) and tert-butyl N-[(4-aminophenyl)methyl]carbamate (2.3 g, 10.4 mmol) in DMSO (25 mL) was added DIEA (4.0 g, 31.1 mmol). The mixture was stirred at 80° C. for 12 hr. The reaction mixture was quenched with H₂O (50 mL) at 25° C. and extracted with EtOAc (50 mL×3). The combined organic layers were washed with brine (50 mL×2), dried over Na₂SO₄, filtered, and concentrated under reduced pressure to give crude product. The crude product was purified by flash chromatography on silica gel (Eluent of 0˜10% MeOH in CH₂Cl₂) to give tert-butyl N-[[4-[(6-chloro-3-nitro-2-pyridyl)amino]phenyl]methyl]carbamate (2.4 g, yield: 44%) as a red solid. MS: m/z=400.9 [M+Na]+. ¹H NMR (400 MHz, Dimethylsulfoxide-d₆) δ 10.10 (s, 1H), 8.53 (d, J=8.8 Hz, 1H), 7.52 (d, J=8.4 Hz, 2H), 7.40 (t, J=6.0 Hz, 1H), 7.25 (d, J=8.4 Hz, 2H), 6.99 (d, J=8.8 Hz, 1H), 4.13 (d, J=6.0 Hz, 2H), 1.40 (s, 9H).

### Step 2: Tert-butyl N-[[4-[2-(2-amino-3-pyridyl)-5-chloro-imidazo[4,5-b]pyridin-3-yl]phenyl]methyl]carbamate

To a solution of tert-butyl N-[[4-[(6-chloro-3-nitro-2-pyridyl)amino]phenyl]methyl]carbamate (2.0 g, 5.3 mmol) in DMSO (30 mL) and MeOH (15 mL) was added 2-aminopyridine-3-carbaldehyde (0.7 g, 5.8 mmol) and Na₂S₂O₄ (1.8 g, 10.6 mmol). The mixture was stirred at 100° C. for 16 hr. The reaction mixture was quenched with H₂O (50 mL) at 25° C., and extracted with EtOAc (100 mL×3). The combined organic layers were washed with brine (100 mL×2), dried over Na₂SO₄, filtered, and concentrated under reduced pressure to give a crude product. The crude product was purified by flash chromatography on silica gel (Eluent of 0˜10% MeOH in CH₂Cl₂) to give tert-butyl N-[[4-[2-(2-amino-3-pyridyl)-5-chloro-imidazo[4,5-b]pyridin-3-yl]phenyl]methyl]carbamate (1.4 g, yield: 51%) as a yellow solid. MS: m/z=451.0 [M+H]⁺. ¹H NMR (400 MHz, Dimethylsulfoxide-d₆) δ 8.26 (d, J=8.4 Hz, 1H), 7.99 (dd, J=4.8, 1.6 Hz, 1H), 7.50-7.43 (m, 2H), 7.38 (s, 4H), 7.21 (dd, J=7.6, 1.6 Hz, 1H), 6.91 (br s, 2H), 6.40 (dd, J=7.6, 4.8 Hz, 1H), 4.22 (d, J=6.0 Hz, 2H), 1.41 (s, 9H).

### Step 3: Tert-butyl N-[[4-[2-(2-amino-3-pyridyl)-5-phenyl-imidazo[4,5-b]pyridin-3-yl]phenyl]methyl]carbamate

To a solution of tert-butyl N-[[4-[2-(2-amino-3-pyridyl)-5-chloro-imidazo[4,5-b]pyridin-3-yl]phenyl]methyl]carbamate (500 mg, 1.1 mmol) and phenylboronic acid (270 mg, 2.2 mmol,) in 1,4-dioxane (5 mL) and H₂O (1 mL) was added Pd(dppf)Cl₂ (81.1 mg, 111 μmol) and Cs₂CO₃ (1.1 g, 3.3 mmol). The mixture was degassed and purged with N₂ three times and then stirred at 80° C. for 16 hr under N₂ atmosphere. After cooling to 20° C., the reaction was diluted with 10 mL of EtOAc, and the mixture filtered through celite and extracted with H₂O (10 mL×3), the combined organic layers were washed with brine (15 mL×3), dried over Na₂SO₄, filtered and concentrated under reduced pressure to give a residue. The crude product was purified by flash chromatography on silica gel (Eluent of 10˜35% EtOAc in petroleum ether) to give tert-butyl N-[[4-[2-(2-amino-3-pyridyl)-5-phenyl-imidazo[4,5-b]pyridin-3-yl]phenyl]methyl]carbamate (126 mg, yield: 21%) as a brown solid. MS: m/z=493.2 [M+H]⁺.

### Step 4: 3-(3-(4-(Aminomethyl)phenyl)-5-phenyl-3H-imidazo[4,5-b]pyridin-2-yl)pyridin-2-amine

To a solution of tert-butyl N-[[4-[2-(2-amino-3-pyridyl)-5-phenyl-imidazo[4,5-b]pyridin-3-yl]phenyl]methyl]carbamate (126 mg, 256 μmol) in HCl/1,4-dioxane (4 M, 1 mL). The mixture was stirred at 25° C. for 2 hr. The solvent was removed under reduced pressure to give a crude product (84 mg, yield: 84%). The crude product was purified by prep-HPLC (column: Welch Xtimate C18 150×25 mm×5 μm; mobile phase: [water (HCl)-ACN]; B %: 5%-35%, 8 min) to give the desired product (HCl salt). The product was diluted with aqueous NaHCO₃ (10 mL) and extracted with DCM (10 mL×3). The combined organic layers were washed with brine (10 mL), dried over Na₂SO₄, filtered, and concentrated under reduced pressure to give 3-(3-(4-(aminomethyl)phenyl)-5-phenyl-3H-imidazo[4,5-b]pyridin-2-yl)pyridin-2-amine (Intermediate 4, 32.2 mg, yield: 84%) as a light-yellow solid. MS: m/z=393.1 [M+H]⁺. ¹H NMR (400 MHz, Dimethylsulfoxide-d₆) δ 8.26 (d, J=8.4 Hz, 1H), 8.04-7.96 (m, 4H), 7.54-7.49 (m, 2H), 7.49-7.43 (m, 2H), 7.43-7.35 (m, 3H), 7.21 (dd, J=7.6, 1.6 Hz, 1H), 6.98 (br s, 2H), 6.41 (dd, J=7.6, 4.8 Hz, 1H), 3.82 (s, 2H).

### Intermediate 5: N-(3-(3-(4-(aminomethyl)phenyl)-2-(2-aminopyridin-3-yl)-3H-imidazo[4,5-b]pyridin-5-yl)phenyl)acetamide

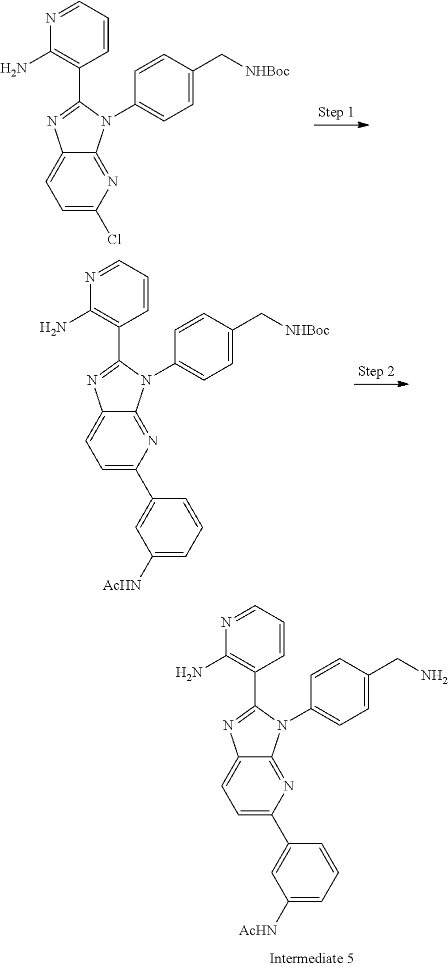

### Step 1: Tert-butyl 4-(5-(3-acetamidophenyl)-2-(2-aminopyridin-3-yl)-3H-imidazo[4,5-b]pyridin-3-yl)benzylcarbamate

To a solution of tert-butyl N-[[4-[2-(2-amino-3-pyridyl)-5-chloro-imidazo[4,5-b]pyridin-3-yl]phenyl]methyl]carbamate (3.5 g, 7.8 mmol) and (3-acetamidophenyl)boronic acid (2.8 g, 15.5 mmol) in 1,4-dioxane (30 mL) and H₂O (6 mL) was added Pd(dppf)Cl₂ (568 mg, 776 μmol) and Cs₂CO₃ (7.6 g, 23.3 mmol). The mixture was was degassed and purged with N₂ three times and then stirred at 80° C. for 16 hr under N₂ atmosphere. The reaction mixture was quenched with of H₂O (50 mL) at 25° C. and extracted with EtOAc (50 mL×3). The combined organic layers were washed with brine (50 mL×2), dried over Na₂SO₄, filtered, and concentrated under reduced pressure to give tert-butyl N-[[4-[5-(3-acetamidophenyl)-2-(2-amino-3-pyridyl)imidazo[4,5-b]pyridin-3-yl]phenyl]methyl]carbamate (2.43 g, yield: 57%), which was directly used to the next step without further purification. MS: m/z=550.1 [M+H]⁺.

### Step 2: N-(3-(3-(4-(Aminomethyl)phenyl)-2-(2-aminopyridin-3-yl)-3H-imidazo[4,5-b]pyridin-5-yl)phenyl)acetamide

To a solution of tert-butyl N-[[4-[5-(3-acetamidophenyl)-2-(2-amino-3-pyridyl)imidazo[4,5-b]pyridin-3-yl]phenyl]methyl]carbamate (8.0 g, 14.6 mmol) in HCl/1,4-dioxane (4 M, 20 mL). The mixture was stirred at 25° C. for 2 hr. Then the solvent was removed under reduced pressure to give a crude product (6.4 g, HCl salt, yield: 90%). 250 mg of the HCl salt was diluted with aqueous NaHCO₃ (10 mL) and extracted with CH₂Cl₂ (10 mL×3). The combined organic layers were washed with brine (10 mL), dried over Na₂SO₄, filtered and concentrated under reduced pressure to give N-(3-(3-(4-(aminomethyl)phenyl)-2-(2-aminopyridin-3-yl)-3H-imidazo[4,5-b]pyridin-5-yl)phenyl)acetamide (Intermediate 5, 86.7 mg, yield: 90%) as a light-yellow solid. MS: m/z=450.0 [M+H]⁺. ¹H NMR (400 MHz, Dimethylsulfoxide-d₆) δ 10.08 (s, 1H), 8.26 (d, J=8.4 Hz, 1H), 8.10 (s, 1H), 8.00 (dd, J=4.8, 1.6 Hz, 1H), 7.84 (d, J=8.4 Hz, 1H), 7.68 (d, J=8.0 Hz, 1H), 7.65 (d, J=8.0 Hz, 1H), 7.53-7.48 (m, 2H), 7.45-7.35 (m, 3H), 7.20 (dd, J=7.6, 1.6 Hz, 1H), 6.94 (br s, 2H), 6.41 (dd, J=7.6, 4.8 Hz, 1H), 3.82 (s, 2H), 2.06 (s, 3H).

### Intermediate 6: N-(3-(2-(2-aminopyridin-3-yl)-3-(4-(piperazin-1-ylmethyl)phenyl)-3H-imidazo[4,5-b]pyridin-5-yl)phenyl)acetamide

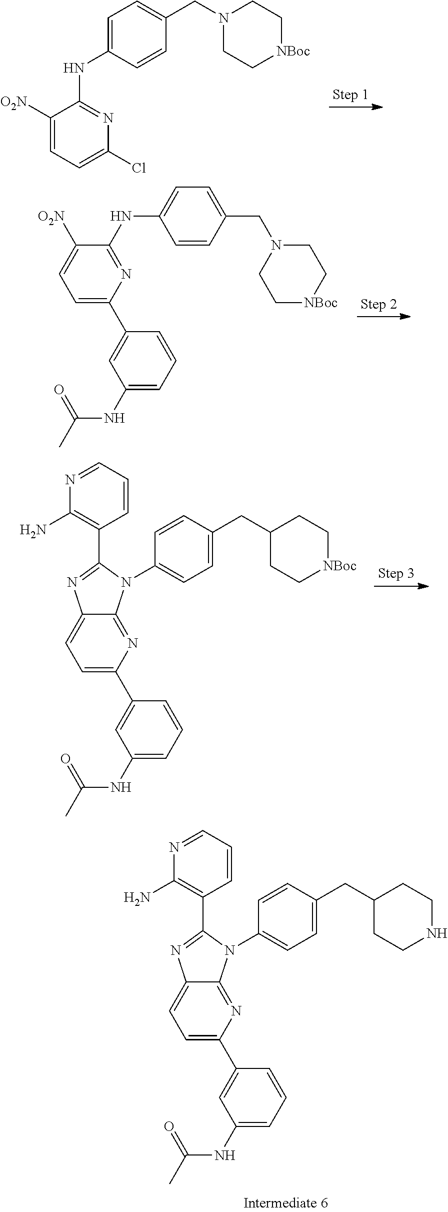

### Step 1: Tert-butyl 4-(4-((6-(3-Acetamidophenyl)-3-nitropyridin-2-yl)amino)benzyl)piperazine-1-carboxylate

To a solution of tert-butyl 4-[[4-[(6-chloro-3-nitro-2-pyridyl)amino]phenyl]methyl]piperazine-1-carboxylate (2.78 g, 5.39 mmol,) and (3-acetamidophenyl)boronic acid (1.93 g, 10.78 mmol) in 1,4-dioxane (30 mL) and H₂O (6 mL) was added K₂CO₃ (2.24 g, 16.2 mmol) and Pd(dppf)Cl₂ (789 mg, 1.08 mmol). The mixture was degassed and purged with N₂ three times and stirred at 60° C. for 16 hr under N₂ atmosphere. The reaction mixture was quenched with H₂O (200 mL) at 25° C., and then extracted with CH₂Cl₂ (60 mL×3). The combined organic layers were washed with brine (100 mL×2), dried over Na₂SO₄, filtered, and concentrated under reduced pressure to give a residue. The residue was purified by column chromatography (Eluent of 50˜100% EtOAc in petroleum ether) to give tert-butyl 4-[[4-[[6-(3-acetamidophenyl)-3-nitro-2-pyridyl]amino]phenyl]methyl]piperazine-1-carboxylate (2.81 g, yield: 83%) as a red solid. MS: m/z=547.1 [M+H]⁺. ¹H NMR (400 MHz, Chloroform-d) δ 10.19 (s, 1H), 8.42 (d, J=8.8 Hz, 1H), 8.19 (br s, 1H), 7.68-7.60 (m, 4H), 7.47 (d, J=9.2 Hz, 1H), 7.34-7.27 (m, 2H), 7.17-7.12 (m, 1H), 3.43 (s, 2H), 3.35 (br t, J=4.8 Hz, 4H), 2.32 (br t, J=4.8 Hz, 4H), 2.11 (s, 3H), 1.36 (s, 9H).

### Step 2: Tert-butyl 4-(4-(5-(3-acetamidophenyl)-2-(2-aminopyridin-3-yl)-3H-imidazo[4,5-b]pyridin-3-yl)benzyl)piperazine-1-carboxylate

To a solution of 2-aminopyridine-3-carbaldehyde (2.3 g, 18.9 mmol) and tert-butyl 4-[[4-[(6-chloro-3-nitro-2-pyridyl)amino]phenyl]methyl]piperazine-1-carboxylate (9.5 g, 17.2 mmol) in DMSO (100 mL) was added Na₂S₂O₄ (8.97 g, 51.5 mmol). The mixture was degassed and purged with N₂ three times and stirred at 60° C. for 16 hr under N₂ atmosphere. The reaction mixture was quenched with H₂O (400 mL) at 25° C., and then extracted with CH₂Cl₂ (400 mL×2). The combined organic layers were washed with brine (300 mL×2), dried over Na₂SO₄, filtered, and concentrated under reduced pressure to give tert-butyl 4-[[4-[5-(3-acetamidophenyl)-2-(2-amino-3-pyridyl)imidazo[4,5-b]pyridin-3-yl]phenyl]methyl]piperazine-1-carboxylate (9.67 g, crude) as a red solid, which was direct used to the next step without further purification. MS: m/z=619.2 [M+H]⁺.

### Step 3: N-(3-(2-(2-aminopyridin-3-yl)-3-(4-(piperazin-1-ylmethyl)phenyl)-3H-imidazo[4,5-b]pyridin-5-yl)phenyl)acetamide

To a solution of tert-butyl 4-[[4-[5-(3-acetamidophenyl)-2-(2-amino-3-pyridyl)imidazo[4,5-b]pyridin-3-yl]phenyl]methyl]piperazine-1-carboxylate (0.4 g, 370 μmol, crude) in HCl/1,4-dioxane (4 M, 5 mL) and 1,4-dioxane (1 mL). The mixture was degassed and purged with N₂ three times and stirred at 25° C. for 4 hr under N₂ atmosphere. The reaction mixture was filtered and the filtered cake was washed with 1,4-dioxane (10 mL×2) and then concentrated under reduced pressure to give a residue. The residue was purified by prep-HPLC (column: Phenomenex Luna C18 200*40 mm*10 um; mobile phase: [water (HCl)-ACN]; B %: 1%-30%, 10 min) to give N-(3-(2-(2-aminopyridin-3-yl)-3-(4-(piperazin-1-ylmethyl)phenyl)-3H-imidazo[4,5-b]pyridin-5-yl)phenyl)acetamide (Intermediate 6, 244 mg HCl salt, yield: 68%) as a yellow solid. MS: m/z=519.2 [M+H]⁺. ¹H NMR (400 MHz, Dimethylsulfoxide-d₆) δ 10.05 (s, 1H), 8.27 (d, J=8.4 Hz, 1H), 8.15 (s, 1H), 7.99 (dd, J=4.8, 1.6 Hz, 1H), 7.86 (d, J=8.4 Hz, 1H), 7.66 (d, J=8.0 Hz, 2H), 7.50-7.33 (m, 5H), 7.14 (dd, J=7.6, 1.2 Hz, 1H), 7.00 (br s, 2H), 6.37 (dd, J=7.6, 4.8 Hz, 1H), 3.52 (s, 2H), 2.77-2.67 (m, 4H), 2.41-2.27 (m, 4H), 2.05 (s, 3H).

### Intermediate 7: 3-[3-[4-(aminomethyl)phenyl]-6-phenyl-imidazo[4,5-b]pyridin-2-yl]pyridin-2-amine

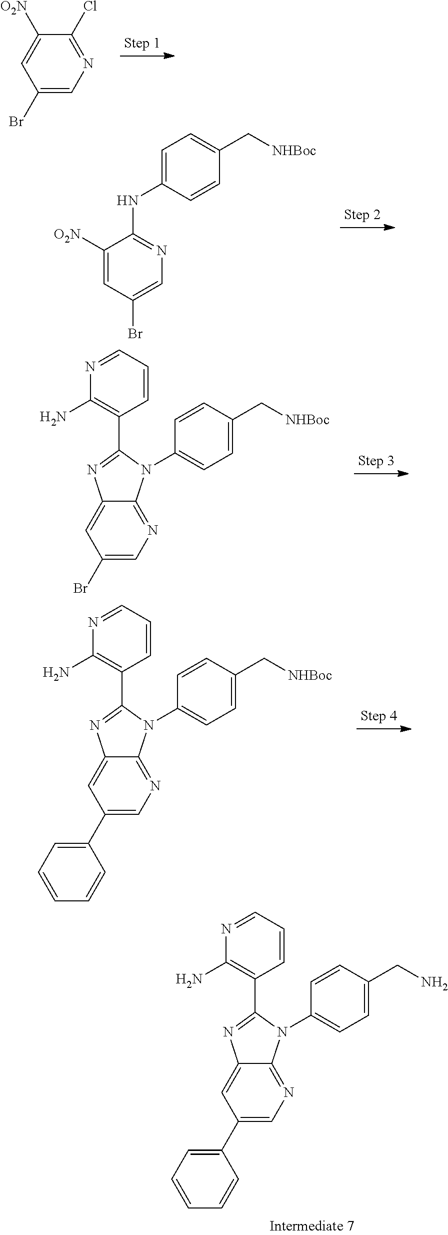

### Step 1: Tert-butyl N-[[4-[(5-bromo-3-nitro-2-pyridyl)amino]phenyl]methyl]carbamate

To a solution of 5-bromo-2-chloro-3-nitro-pyridine (2.1 g, 9.0 mmol) and tert-butyl N-[(4-aminophenyl)methyl]carbamate (2 g, 9.0 mmol) in DMSO (20 mL) was added DIEA (3.5 g, 27.0 mmol). The mixture was stirred at 80° C. for 12 hr. After cooling to 20° C., H₂O (50 mL) was added to the reaction mixture, then extracted with EtOAc (50 mL×2). The combined organic layers were washed with brine (50 mL×2), dried over Na₂SO₄, filtered, and concentrated under reduced pressure to give tert-butyl N-[[4-[(5-bromo-3-nitro-2-pyridyl)amino]phenyl]methyl]carbamate (3.5 g crude, yield: 92%) as a red solid. MS: m/z=367.6, 368.6 [M+H]⁺. ¹H NMR (400 MHz, Chloroform-d)) δ 10.02 (s, 1H), 8.65 (d, J=2.0 Hz, 1H), 8.49 (d, J=2.0 Hz, 1H), 7.55 (d, J=8.4 Hz, 2H), 7.32 (d, J=8.0 Hz, 2H), 4.84 (br s, 1H), 4.32 (d, J=5.2 Hz, 2H), 1.47 (s, 9H).

### Step 2: Tert-butyl N-[[4-[2-(2-amino-3-pyridyl)-6-bromo-imidazo[4,5-b]pyridin-3-yl]phenyl]methyl]carbamate

To a solution of tert-butyl N-[[4-[(5-bromo-3-nitro-2-pyridyl)amino]phenyl]methyl]carbamate (3 g, 7.1 mmol) and 2-aminopyridine-3-carbaldehyde (952 mg, 7.8 mmol) in DMSO (30 mL) and MeOH (15 mL) was added Na₂S₂O₄ (2.5 g, 14.2 mmol). The mixture was stirred at 100° C. for 12 hr. After cooling to 20° C., H₂O (50 mL) was added to the reaction mixture, then the resulting mixture extracted with EtOAc (50 mL×2). The combined organic layers were washed with brine (50 mL×2), dried over Na₂SO₄, filtered and concentrated under reduced pressure to give a residue. The residue was purified by column chromatography (Eluent of 0˜1% MeOH in CH₂Cl₂) to give tert-butyl-N-[[4-[2-(2-amino-3-pyridyl)-6-bromo-imidazo[4,5-b]pyridin-3-yl]phenyl]methyl]carbamate (1.7 g, yield: 36%) as a yellow solid. MS: m/z=495.9, 496.9 [M+H]⁺. ¹H NMR (400 MHz, Chloroform-d) δ 8.40 (d, J=2.0 Hz, 1H), 8.22 (d, J=2.0 Hz, 1H), 8.08 (dd, J=4.8, 2.0 Hz, 1H), 7.46 (d, J=8.4 Hz, 2H), 7.33 (d, J=8.4 Hz, 2H), 7.10 (dd, J=8.0, 2.0 Hz, 1H), 6.63 (br s, 2H), 6.41-6.34 (m, 1H), 4.93 (br s, 1H), 4.42 (d, J=5.6 Hz, 2H), 1.48 (s, 9H).

### Step 3: Tert-butyl N-[[4-[2-(2-amino-3-pyridyl)-6-phenyl-imidazo[4,5-b]pyridin-3-yl]phenyl]methyl]carbamate

To a solution of tert-butyl N-[[4-[2-(2-amino-3-pyridyl)-6-bromo-imidazo[4,5-b]pyridin-3-yl]phenyl]methyl]carbamate (500 mg, 1.0 mmol) and phenylboronic acid (246 mg, 2.0 mmol) in toluene (5 mL) and EtOH (5 mL) was added NaHCO₃ (254 mg, 3.0 mmol) and Pd(PPh₃)₄ (233 mg, 202 μmol). The mixture degassed and purged with N₂ three times, and then the mixture was stirred at 100° C. for 12 hr under N₂ atmosphere. After cooling to 20° C., H₂O (20 mL) was added to the reaction mixture, then extracted with EtOAc (20 mL×2). The combined organic layers were washed with saturated NaHCO₃ solution (20 mL×2), dried over Na₂SO₄, filtered, and concentrated under reduced pressure to give a residue. The residue was purified by silica gel flash chromatography (Eluent of 0˜1% MeOH in CH₂Cl₂) to give tert-butyl N-[[4-[2-(2-amino-3-pyridyl)-6-phenyl-imidazo[4,5-b]pyridin-3-yl]phenyl]methyl]carbamate (340 mg, 62% yield) as a brown solid. MS: m/z=493.1 [M+H]⁺. 1H NMR (400 MHz, Dimethylsulfoxide-d₆)) δ 8.61 (d, J=2.0 Hz, 1H), 8.45 (d, J=2.0 Hz, 1H), 8.01-7.99 (m, 1H), 7.77 (d, J=7.2 Hz, 2H), 7.63-7.59 (m, 4H), 7.54-7.52 (m, 2H), 7.39 (d, J=5.2 Hz, 2H), 7.23 (dd, J=7.6, 2.0 Hz, 1H), 7.04 (br s, 2H), 6.40 (dd, J=7.6, 5.2 Hz, 1H), 4.21 (d, J=6.0 Hz, 2H), 1.40 (s, 9H).

### Step 4. 3-[3-[4-(aminomethyl)phenyl]-6-phenyl-imidazo[4,5-b]pyridin-2-yl]pyridin-2-amine

To a solution of tert-butyl N-[[4-[2-(2-amino-3-pyridyl)-6-phenyl-imidazo[4,5-b]pyridin-3-yl]phenyl]methyl]carbamate (340 mg, 690 μmol, 1.0 eq) in 1,4-dioxane (5 mL) was added HCl/dioxane (5 mL). The mixture was stirred at 25° C. for 1 hr. The reaction mixture was concentrated under reduced pressure to give the crude product (297 mg, HCl salt, yield: 96%). The residue was purified by prep-HPLC (column: Welch Xtimate C₁₈ 150×25 mm×5 μm; mobile phase: [water (HCl)-ACN]; B %: 3%-33%, 8 min) and dissociated with NaHCO₃ to give 3-[3-[4-(aminomethyl)phenyl]-6-phenyl-imidazo[4,5-b]pyridin-2-yl]pyridin-2-amine (Intermediate 7, 83.8 mg, yield: 96%) as an off-white solid. MS: m/z=393.1 [M+H]⁺. ¹H NMR (400 MHz, Dimethylsulfoxide-d₆)) δ 8.62 (d, J=2.0 Hz, 1H), 8.46 (d, J=2.0 Hz, 1H), 8.00 (dd, J=4.8, 2.0 Hz, 1H), 7.78 (d, J=7.2 Hz, 2H), 7.56-7.46 (m, 5H), 7.45-7.35 (m, 4H), 7.26 (dd, J=8.0, 2.0 Hz, 1H), 7.04 (s, 2H), 6.44-6.39 (m, 1H), 3.80 (s, 2H).

### Intermediate 8: 3-(6-Phenyl-3-(4-(piperazin-1-ylmethyl)phenyl)-3H-imidazo[4,5-b]pyridin-2-yl)pyridin-2-amine

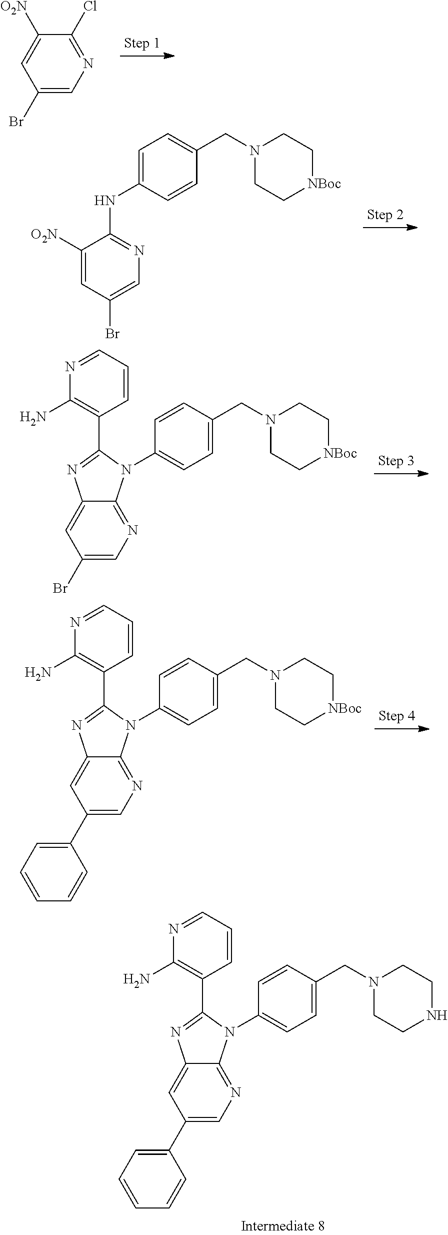

### Step 1: Tert-butyl 4-(4-((5-bromo-3-nitropyridin-2-yl)amino)benzyl)piperazine-1-carboxylate

To a solution of 5-bromo-2-chloro-3-nitro-pyridine (10 g, 42.1 mmol) in DMSO (100 mL) was added tert-butyl 4-[(4-aminophenyl)methyl]piperazine-1-carboxylate (11 g, 30.2 mmol) and DIEA (11.7 g, 94.8 mmol). The mixture was stirred at 80° C. for 12 hr. After cooling to 20° C., the reaction mixture was poured into H₂O (100 mL). The aqueous layer was extracted with EtOAc (200 mL×3). The combined organic layers were washed with brine (200 mL×3). The combined organic layers were dried over anhydrous Na₂SO₄, filtered, and concentrated to dryness. The crude product was purified by flash chromatography on silica gel (Elute of 10-50% EtOAc in petroleum ether) to give tert-butyl 4-[[4-[(5-bromo-3-nitro-2-pyridyl)amino]phenyl]methyl]piperazine-1-carboxylate (10.7 g, yield: 43%) as a brown solid. MS: m/z=493.8 [M+H]⁺. ¹H NMR (400 MHz, Dimethylsulfoxide-d₆) δ 9.93 (s, 1H), 8.67 (d, J=2.4 Hz, 1H), 8.60 (d, J=2.4 Hz, 1H), 8.50 (dd, J=10.0, 2.4 Hz, 1H), 7.54 (d, J=8.4 Hz, 2H), 7.29 (d, J=8.0 Hz, 1H), 3.46 (s, 2H), 3.32-3.28 (m, 4H), 2.32-2.30 (m, 4H), 1.39 (s, 9H)

### Step 2: Tert-butyl 4-(4-(2-(2-aminopyridin-3-yl)-6-bromo-3H-imidazo[4,5-b]pyridin-3-yl)benzyl)piperazine-1-carboxylate

To a solution of tert-butyl 4-[[4-[(5-bromo-3-nitro-2-pyridyl)amino]phenyl]methyl]piperazine-1-carboxylate (10.7 g, 21.6 mmol) in DMSO (60 mL) and methanol (30 mL) was added 2-aminopyridine-3-carbaldehyde (3.2 g, 26 mmol) and Na₂S₂O₄ (7.5 g, 43.3 mmol). Then the reaction mixture was stirred at 100° C. for 16 hr. After cooling to 25° C., the reaction mixture was diluted with H₂O (100 mL) and extracted with CH₂Cl₂ (100 mL×3) and brine (100 mL×2 times). The organic phase was separated and dried over Na₂SO₄, filtered, and concentrated under reduced pressure to give a residue. The crude product tert-butyl 4-[[4-[2-(2-amino-3-pyridyl)-6-bromo-imidazo[4,5-b]pyridin-3-yl]phenyl]methyl]piperazine-1-carboxylate (7.6 g, yield: 62%) was obtained as a red oil. MS: m/z=565.9 [M+H]⁺.

### Step 3: Tert-butyl 4-(4-(2-(2-aminopyridin-3-yl)-6-phenyl-3H-imidazo[4,5-b]pyridin-3-yl)benzyl)piperazine-1-carboxylate

To a solution of tert-butyl 4-[[4-[2-(2-amino-3-pyridyl)-6-bromo-imidazo[4,5-b]pyridin-3-yl]phenyl]methyl]piperazine-1-carboxylate (5.8 g, 10.3 mmol) and phenylboronic acid (2.5 g, 20.6 mmol) in 1,4-dioxane (100 mL) and H₂O (10 mL) was added Pd(dppf)Cl₂ (752 mg, 1.03 mmol) and Cs₂CO₃ (10 g, 30.8 mmol). The mixture was degassed and purged with N₂ three times, and then the mixture was stirred at 80° C. for 16 hr under N₂ atmosphere. After cooling to 25° C., the reaction mixture was poured into H₂O (100 mL). The aqueous layer was extracted with EtOAc (100 mL×3). The combined organic layers were washed with brine (100 mL×3). The combined organic layers were dried over anhydrous Na₂SO₄, filtered, and concentrated to dryness. The crude product tert-butyl 4-[[4-[2-(2-amino-3-pyridyl)-6-phenyl-imidazo[4,5-b]pyridin-3-yl]phenyl]methyl]piperazine-1-carboxylate (2.54 g, yield: 44%) as a brown oil. MS: m/z=562.4 [M+H]⁺.

### Step 4: 3-(6-Phenyl-3-(4-(piperazin-1-ylmethyl)phenyl)-3H-imidazo[4,5-b]pyridin-2-yl)pyridin-2-amine

A solution of tert-butyl 4-[[4-[2-(2-amino-3-pyridyl)-6-phenyl-imidazo[4,5-b]pyridin-3-yl]phenyl]methyl]piperazine-1-carboxylate (10 g, 17.80 mmol, 1 eq) in HCl/1,4-dioxane (4 M, 100 mL) was stirred at 25° C. for 1 hr. The reaction mixture was filtered and concentrated under reduced pressure to give the crude product. The crude product was triturated with MeOH (30 mL) at 25° C. for 30 min. The suspension was filtered, the filter cake washed with MeOH (20 mL) and concentrated under reduced pressure to give 3-[6-phenyl-3-[4-(piperazin-1-ylmethyl)phenyl]imidazo[4,5-b]pyridin-2-yl]pyridin-2-amine (Intermediate 8, 3.7 g, yield: 42%) as a yellow solid. MS: m/z=462.2 [M+H]⁺. ¹H NMR (400 MHz, Dimethylsulfoxide-d₆) δ 8.63 (d, J=2.0 Hz, 1H), 8.46 (d, J=2.0 Hz, 1H), 8.00 (dd, J=4.8, 1.6 Hz, 1H), 7.81-7.75 (m, 2H), 7.52 (dd, J=7.6, 7.6 Hz, 2H), 7.47-7.38 (m, 5H), 7.19 (dd, J=7.6, 1.6 Hz, 1H), 7.06 (br s, 2H), 6.39 (dd, J=7.6, 4.8 Hz, 1H), 3.51 (s, 2H), 3.33-3.27 (m, 1H), 2.71 (br t, J=4.4 Hz, 4H), 2.32 (s, 4H).

### Intermediate 9: 3-[3-[4-[(4-Amino-1-piperidyl)methyl]phenyl]-6-phenyl-imidazo[4,5-b]pyridin-2-yl]pyridin-2-amine

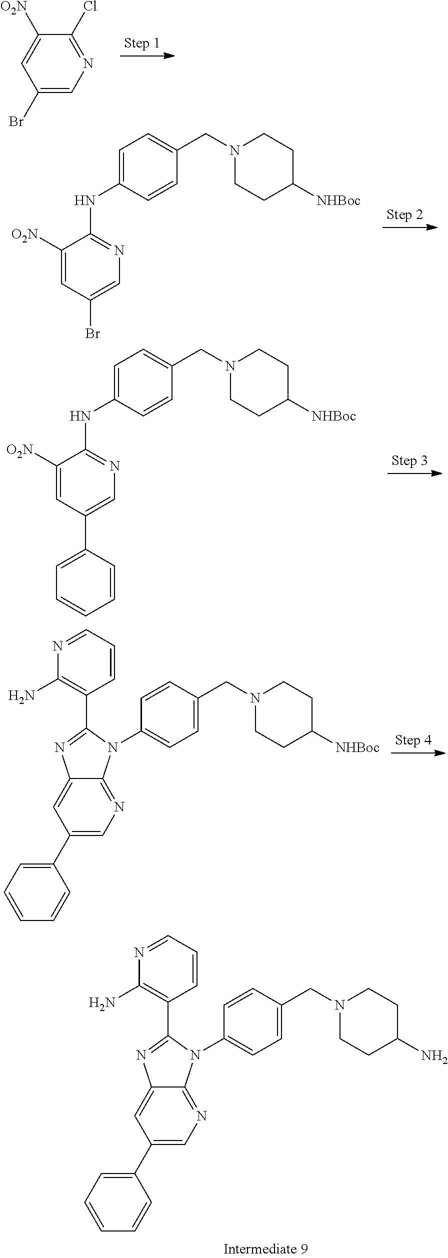

### Step 1: Tert-butyl N-[1-[[4-[(5-bromo-3-nitro-2-pyridyl)amino]phenyl]methyl]-4-piperidyl]carbamate

A mixture of 5-bromo-2-chloro-3-nitro-pyridine (17.1 g, 72 mmol), tert-butyl N-[1-[(4-aminophenyl)methyl]-4-piperidyl]carbamate (22 g, 72 mmol), DIEA (27.9 g, 216 mmol) in DMSO (200 mL) was stirred at 80° C. for 16 hr. After cooling to 25° C., the mixture was extracted with EtOAc (250 mL×3). The combined organic layers were washed with brine (200 mL×2), dried over anhydrous Na₂SO₄, filtered, and concentrated to give tert-butyl N-[1-[[4-[(5-bromo-3-nitro-2-pyridyl)amino]phenyl]methyl]-4-piperidyl]carbamate crude product as black brown solid (32 g). MS: m/z=506.9, 507.9 [M+H]⁺. ¹H NMR (400 MHz, Chloroform-d) δ 10.04 (s, 1H), 8.64 (d, J=2.0 Hz, 1H), 8.49 (d, J=2.4 Hz, 1H), 7.53 (d, J=8.4 Hz, 2H), 7.33 (d, J=8.4 Hz, 2H), 4.34 (d, J=3.2 Hz, 1H), 3.48 (s, 2H), 3.46-3.35 (m, 1H), 2.82 (br d, J=12.0 Hz, 2H), 2.10 (t, J=10.8 Hz, 2H), 1.91 (br d, J=11.2 Hz, 2H), 1.44 (s, 9H).

### Step 2: Tert-butyl N-[1-[[4-[(3-nitro-5-phenyl-2-pyridyl)amino]phenyl]methyl]-4-piperidyl]carbamate

A mixture of tert-butyl N-[1-[[4-[(5-bromo-3-nitro-2-pyridyl)amino]phenyl]methyl]-4-piperidyl]carbamate (20 g, 39.5 mmol), phenylboronic acid (4.8 g, 39.5 mmol), K₂CO₃ (16.4 g, 118.5 mmol), Pd(dppf)Cl₂ (1.4 g, 2.0 mmol) in 1,4-dioxane (250 mL) and H₂O (50 mL) was degassed and purged with N₂ three times, and then the mixture was stirred at 80° C. for 16 hr under N₂ atmosphere. After cooling to 25° C., the reaction mixture was filtered, then to the liquid added H₂O (100 mL) and EtOAc (450 mL). The organic phase was separated, washed with brine (100 mL×3), dried over Na₂SO₄, filtered, and concentrated under reduced pressure to give the residue. The residue was purified by silica gel flash chromatography (Eluent of 3˜4% MeOH in CH₂Cl₂) to give tert-butyl-[1-[[4-[(3-nitro-5-phenyl-2-pyridyl)amino]phenyl]methyl]-4-piperidyl]carbamate (8.6 g, yield: 43%) as a red brown soild. MS: m/z=504.2 [M+H]⁺. ¹H NMR (400 MHz, Chloroform-d) δ 10.14 (s, 1H), 8.75 (dd, J=10.0, 2.0 Hz, 2H), 7.62 (d, J=8.0, Hz, 2H), 7.57 (d, J=7.2, Hz, 2H), 7.49 (dd, J=8.0, 8.0 Hz, 2H), 7.43-7.37 (m, 3H), 4.43 (br s, 1H), 3.50 (br s, 2H), 3.49-3.41 (m, 1H), 2.85-2.81 (m, 2H), 2.15-2.08 (m, 2H), 1.94-1.91 (m, 2H), 1.51-1.45 (m, 2H), 1.44 (s, 9H).

### Step 3. Tert-butyl N-[1-[[4-[2-(2-amino-3-pyridyl)-6-phenyl-imidazo[4,5-b]pyridin-3-yl]phenyl]methyl]-4-piperidyl]carbamate

A mixture of tert-butyl N-[1-[[4-[(3-nitro-5-phenyl-2-pyridyl)amino]phenyl]methyl]-4-piperidyl]carbamate (2.2 g, 4.4 mmol), 2-aminopyridine-3-carbaldehyde (694 mg, 5.7 mmol), Na₂S₂O₄ (1.5 g, 8.7 mmol) in DMSO (100 mL) was degassed and purged with N₂ three times, and then the mixture was stirred at 100° C. for 16 hr under N₂ atmosphere. After cooling to 25° C., the reaction mixture was filtered, then to the liquid added H₂O (30 mL) and EtOAc (45 mL). The organic phase was separated, washed with brine (10 mL×3), dried over Na₂SO₄, filtered and concentrated under reduced pressure to give the residue. The residue was purified by silica gel flash chromatography (Eluent of 5˜6% EtOAc in petroleum ether) to give tert-butyl N-[1-[[4-[2-(2-amino-3-pyridyl)-6-phenyl-imidazo[4,5-b]pyridin-3-yl]phenyl]methyl]-4-piperidyl]carbamate (700 mg, yield: 28%) as a black brown soild. MS: m/z=576.2 [M+H]⁺. ¹H NMR (400 MHz, Chloroform-d) δ 8.63 (d, J=2.0 Hz, 1H), 8.27 (d, J=2.0 Hz, 1H), 8.07 (dd, J=5.2, 2.0 Hz, 1H), 7.67-7.62 (m, 2H), 7.54-7.47 (m, 4H), 7.44-7.39 (m, 1H), 7.36 (d, J=8.4 Hz, 2H), 7.09 (dd, J=8.0, 2.0 Hz, 1H), 6.65 (br s, 2H), 6.35 (dd, J=8.0, 4.8 Hz, 1H), 4.45 (br s, 1H), 3.58 (s, 2H), 3.55-3.46 (m, 1H), 2.92-2.77 (m, 2H), 2.16 (br t, J=10 Hz, 2H), 1.95 (br d, J=11.2 Hz, 2H), 1.63-1.56 (m, 2H), 1.45 (s, 9H).

### Step 4: 3-[3-[4-[(4-Amino-1-piperidyl)methyl]phenyl]-6-phenyl-imidazo[4,5-b]pyridin-2-yl]pyridin-2-amine

A mixture of tert-butyl N-[1-[[4-[2-(2-amino-3-pyridyl)-6-phenyl-imidazo[4,5-b]pyridin-3-yl]phenyl]methyl]-4-piperidyl]carbamate (2.4 g, 4.17 mmol) in HCl/1,4-dioxane (4 M, 20 mL) and MeOH (4 mL) was stirred at 25° C. for 2 hr. The reaction mixture was filtered to give residue (2 g HCl salt, yield: 94.3%). 100 mg of the residue was purified by prep-HPLC (column: Welch Ultimate C18 150×25 mm×Sum; mobile phase: [water (FA)-ACN]; B %: 0% to 25%, 10 min) to give 3-[3-[4-[(4-amino-1-piperidyl)methyl]phenyl]-6-phenyl-imidazo[4,5-b]pyridin-2-yl]pyridin-2-amine (Intermediate 9, 26.6 mg, 2HCOOH salt, yield: 94%) as a yellow solid. MS: m/z=476.1 [M+H]⁺. ¹H NMR (400 MHz, Dimethylsulfoxide-d₆) δ 8.63 (d, J=2.0 Hz, 1H), 8.47 (d, J=2.0 Hz, 1H), 8.34 (s, 2H), 8.00 (dd, J=4.8, 1.6 Hz, 1H), 7.78 (d, J=7.6 Hz, 2H), 7.53 (dd, J=7.6, 7.6 Hz, 2H), 7.47-7.39 (m, 5H), 7.20 (dd, J=7.6, 1.6 Hz, 1H), 7.04 (br s, 2H), 6.39 (dd, J=7.6, 4.8 Hz, 1H), 3.55 (s, 2H), 2.98-2.88 (m, 1H), 2.87-2.80 (m, 2H), 2.09-1.97 (m, 2H), 1.90-1.80 (m, 2H), 1.57-1.44 (m, 2H).

### Intermediate 10: 3-[5-Phenyl-3-[4-(piperazin-1-ylmethyl)phenyl]imidazo[4,5-b]pyridin-2-yl]pyridin-2-amine

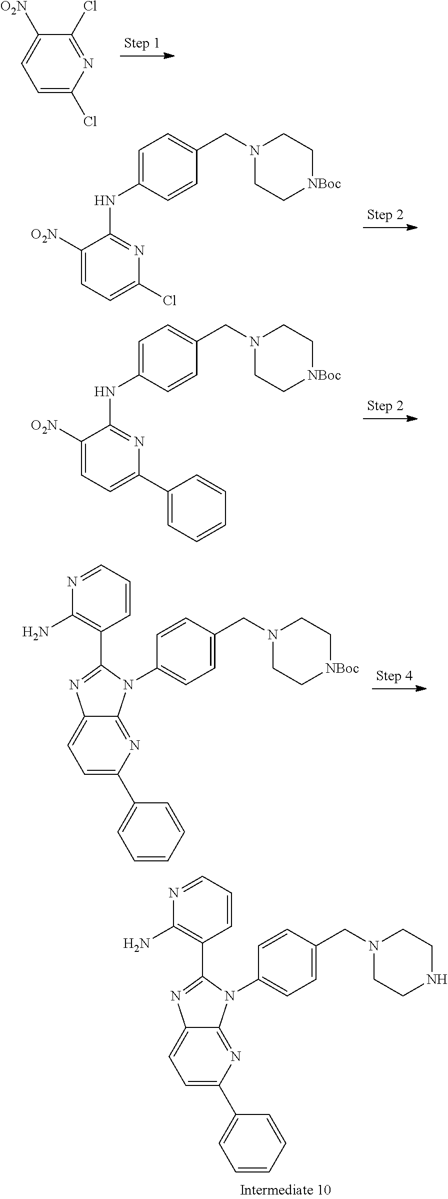

### se N a b4 In-erme eN s 10

### Step 1: Tert-butyl 4-[[4-[(6-chloro-3-nitro-2-pyridyl)amino]phenyl]methyl]piperazine-1-carboxylate

To a solution of 2,6-dichloro-3-nitro-pyridine (5.0 g, 25.9 mmol) in 1,4-dioxane (50 mL) was added DIEA (6.7 g, 51.8 mmol) and tert-butyl 4-[(4-aminophenyl)methyl]piperazine-1-carboxylate (10.8 g, 25.9 mmol). The mixture was stirred at 60° C. for 12 hr. The reaction mixture was diluted 50 mL of H₂O, and the aqueous phase extracted with EtOAc (100 mL×3). The combined organic layers were washed with brine (50 mL×2), dried over Na₂SO₄, filtered, and concentrated under reduced pressure to give a residue. The residue was purified by silica gel flash chromatography (Eluent of 0˜50% EtOAc in petroleum ether) to give tert-butyl 4-[[4-[(6-chloro-3-nitro-2-pyridyl)amino]phenyl]methyl]piperazine-1-carboxylate (6.1 g, yield: 53%) as a yellow solid. MS: m/z=447.9 [M+H]⁺. ¹H NMR (400 MHz, Chloroform-d) δ 10.27 (s, 1H), 8.46 (d, J=8.8 Hz, 1H), 7.61 (d, J=8.4 Hz, 2H), 7.36 (d, J=8.4 Hz, 2H), 6.80 (d, J=8.4 Hz, 1H), 3.52 (s, 2H), 3.48-3.37 (m, 4H), 2.45-2.32 (m, 4H), 1.46 (s, 9H).

### Step 2. Tert-butyl 4-[[4-[(3-nitro-6-phenyl-2-pyridyl)amino]phenyl]methyl]piperazine-1-carboxylate

To a solution of tert-butyl 4-[[4-[(6-chloro-3-nitro-2-pyridyl)amino]phenyl]methyl]piperazine-1-carboxylate (1.0 g, 2.23 mmol) and phenylboronic acid (544 mg, 4.47 mmol) in 1,4-dioxane (10 mL) and H₂O (2 mL) was added Pd(dppf)Cl₂ (327 mg, 0.446 mmol) and K₂CO₃ (926 mg, 6.7 mmol). The mixture was stirred at 60° C. for 4 hr. The reaction mixture was added with 50 mL of H₂O, and the aqueous phase extracted with EtOAc (100 mL×3). The combined organic layers were washed with brine (50 mL×2), dried over Na₂SO₄, filtered, and concentrated under reduced pressure to give a residue. The residue was purified by silica gel flash chromatography (Eluent of 0˜50% EtOAc in petroleum ether) to give tert-butyl 4-[[4-[(3-nitro-6-phenyl-2-pyridyl)amino]phenyl]methyl]piperazine-1-carboxylate (1.0 g, yield: 92%) as a red solid. MS: m/z=490.1 [M+H]⁺. ¹H NMR (400 MHz, Chloroform-d) δ 10.31 (s, 1H), 8.59 (d, J=8.8 Hz, 1H), 8.06-8.04 (m, 2H), 7.73 (d, J=8.4 Hz, 2H), 7.52-7.44 (m, 3H), 7.37 (d, J=8.4 Hz, 2H), 7.3 (d, J=8.4 Hz, 1H), 3.54 (s, 2H), 3.52-3.37 (m, 4H), 2.49-2.37 (m, 4H), 1.46 (s, 9H).

### Step 3: Tert-butyl 4-[[4-[2-(2-amino-3-pyridyl)-5-phenyl-imidazo[4,5-b]pyridin-3-yl]phenyl]methyl]piperazine-1-carboxylate

To a solution of 2-aminopyridine-3-carbaldehyde (269 mg, 2.21 mmol) and tert-butyl 4-[[4-[(3-nitro-6-phenyl-2-pyridyl)amino]phenyl]methyl]piperazine-1-carboxylate (900 mg, 1.84 mmol) in DMSO (10 mL) was added Na₂S₂O₄ (960 mg, 5.52 mmol) at 15° C. The mixture was stirred at 100° C. for 20 hr. The reaction mixture was diluted with H₂O (50 mL), and the aqueous phase extracted with DCM (80 mL×3). The combined organic layers were washed with brine (80 mL×3), dried over Na₂SO₄, filtered, and concentrated under reduced pressure to give a residue. The residue was purified by silica gel flash chromatography (Eluent of 0˜10% MeOH in CH₂Cl₂) to give tert-butyl 4-[[4-[2-(2-amino-3-pyridyl)-5-phenyl-imidazo[4,5-b]pyridin-3-yl]phenyl]methyl]piperazine-1-carboxylate (600 mg, yield: 58%) as a yellow solid. MS: m/z=562.1 [M+H]+. ¹H NMR (400 MHz, Chloroform-d) δ 8.13 (d, J=8.4 Hz, 1H), 8.06 (dd, J=5.2, 2.0 Hz, 1H), 8.03-8.01 (m, 2H), 7.81 (d, J=8.4 Hz, 1H), 7.51-7.49 (m, 2H), 7.48-7.35 (m, 5H), 7.10 (dd, J=9.6, 2.0 Hz, 1H), 6.66 (br s, 2H), 6.36 (dd, J=8.0, 4.0 Hz, 1H), 3.64 (s, 2H), 3.53-3.42 (m, 4H), 2.55-2.42 (m, 4H), 1.47 (s, 9H).

### Step 4: 3-[5-Phenyl-3-[4-(piperazin-1-ylmethyl)phenyl]imidazo[4,5-b]pyridin-2-yl]pyridin-2-amine

A solution of tert-butyl 4-[[4-[2-(2-amino-3-pyridyl)-5-phenyl-imidazo[4,5-b]pyridin-3-yl]phenyl]methyl]piperazine-1-carboxylate (400 mg, 712 μmol) in HCl/1,4-dioxane (4 M, 8 mL) was stirred at 25° C. for 4 hr. The reaction was filtered and concentrated under reduced pressure to give 3-[5-phenyl-3-[4-(piperazin-1-ylmethyl)phenyl]imidazo[4,5-b]pyridin-2-yl]pyridin-2-amine (450 mg, HCl) as a yellow solid, which was used in the next step without further purification. 200 mg of the residue was purified by prep-HPLC (column: Waters xbridge 150*25 mm 10 um; mobile phase: [water(NH₄HCO₃)-ACN]; B %: 19%-649%, 9 min) to give 3-[5-Phenyl-3-[4-(piperazin-1-ylmethyl)phenyl]imidazo[4,5-b]pyridin-2-yl]pyridin-2-amine (Intermediate 10, 49.7 mg) as a yellow solid. MS: m/z=462.2 [M+H]⁺. ¹H NMR (400 MHz, Dimethylsulfoxide-d₆) δ 8.27 (d, J=8.4 Hz, 1H), 8.04-7.97 (m, 4H), 7.48-7.39 (m, 8H), 7.15 (dd, J=8.0, 2.0 Hz, 1H), 7.03 (br s, 2H), 6.38 (dd, J=7.6, 4.8 Hz, 1H), 3.53 (s, 2H), 2.74-2.66 (m, 4H), 2.38-2.27 (m, 4H).

### Intermediate 11: 3-[3-[4-[(4-amino-1-piperidyl)methyl]phenyl]-5-phenyl-imidazo[4,5-b]pyridin-2-yl]pyridin-2-amine

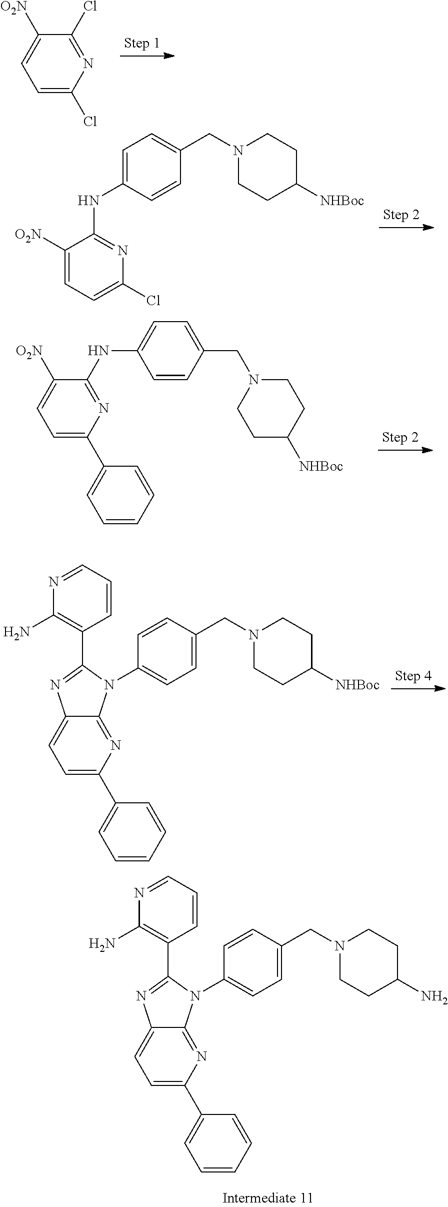

### Step 1: tert-Butyl (1-(4-((6-chloro-3-nitropyridin-2-yl)amino)benzyl)piperidin-4-yl)carbamate

To a solution of 2,6-dichloro-3-nitro-pyridine (3.0 g, 15.5 mmol) and tert-butyl N-[1-[(4-aminophenyl)methyl]-4-piperidyl]carbamate (4.8 g, 15.6 mmol) in 1,4-dioxane (100 mL) was added DIEA (6.0 g, 46.6 mmol). The mixture was stirred at 50° C. for 12 hr. The reaction mixture was diluted with H₂O (50 mL) and extracted with EtOAc (30 mL×2). The combined organic layers were washed with brine (50 mL), dried over Na₂SO₄, filtered, and concentrated under reduced pressure to give a residue. The residue was purified by silica gel flash chromatography (Eluent of 0˜5% MeOH in CH₂Cl₂) to give tert-butyl N-[1-[[4-[(6-chloro-3-nitro-2-pyridyl)amino]phenyl]methyl]-4-piperidyl]carbamate (4.6 g, yield: 64%) as an orange solid. MS: m/z=462.1 [M+H]⁺. ¹H NMR (400 MHz, Chloroform-d) δ 10.26 (s, 1H), 8.46 (d, J=8.8 Hz, 1H), 7.60 (d, J=8.8 Hz, 2H), 7.34 (d, J=8.4 Hz, 2H), 6.79 (d, J=8.4 Hz, 1H), 4.42 (br s, 1H), 3.48 (s, 2H), 3.48-3.39 (m, 1H), 2.82 (br d, J=10.8 Hz, 2H), 2.10 (br t, J=10.8 Hz, 2H), 1.92 (br d, J=10.8 Hz, 2H), 1.50-1.40 (m, 2H). 1.44 (s, 9H).

### Step 2: tert-Butyl (1-(4-((3-nitro-6-phenylpyridin-2-yl)amino)benzyl)piperidin-4-yl)carbamate

A mixture of tert-butyl N-[1-[[4-[(6-chloro-3-nitro-2-pyridyl)amino]phenyl]methyl]-4-piperidyl]carbamate (1.0 g, 2.20 mmol), phenylboronic acid (528 mg, 4.30 mmol), Pd(dppf)Cl₂ (158 mg, 0.216 mmol) and K₂CO₃ (898 mg, 6.50 mmol) in 1,4-dioxane (10 mL) and H₂O (2 mL) was degassed and purged with N₂ three times, and then the mixture was stirred at 100° C. for 12 hr under N₂ atmosphere. After cooling to 25° C., the reaction mixture was diluted with H₂O and extracted with DCM (50 mL×2). The combined organic layers were washed with brine (50 mL), dried over Na₂SO₄, filtered, and concentrated under reduced pressure to give a residue. The residue was purified by silica gel flash chromatography (Eluent of 0˜7% MeOH in CH₂Cl₂) to give tert-butyl N-[1-[[4-[(3-nitro-6-phenyl-2-pyridyl)amino]phenyl]methyl]-4-piperidyl]carbamate (1.1 g, yield: 96%) as a yellow solid. MS: m/z=504.1 [M+H]+. ¹H NMR (400 MHz, Chloroform-d) δ 10.31 (s, 1H), 8.59 (d, J=8.8 Hz, 1H), 8.09-8.02 (m, 2H), 7.72 (d, J=8.4 Hz, 2H), 7.51-7.47 (m, 3H), 7.36 (d, J=8.8 Hz, 1H), 7.30 (d, J=8.8 Hz, 1H), 4.52-4.37 (m, 1H), 3.52 (s, 2H), 3.51-3.42 (m, 1H), 2.85 (br d, J=11.2 Hz, 2H), 2.12 (br t, J=10.8 Hz, 2H), 1.93 (br d, J=11.0 Hz, 2H), 1.50-1.40 (m, 2H). 1.44 (s, 9H).

### Step 3: tert-Butyl N-[1-[[4-[2-(2-amino-3-pyridyl)-5-phenyl-imidazo[4,5-b]pyridin-3-yl]phenyl]methyl]-4-piperidyl]carbamate

To a solution of tert-butyl N-[1-[[4-[(3-nitro-6-phenyl-2-pyridyl)amino]phenyl]methyl]-4-piperidyl]carbamate (200 mg, 0.397 mmol), 2-aminopyridine-3-carbaldehyde (53.4 mg, 0.437 mmol) and Na₂S₂O₄ (207 mg, 1.2 mmol) in DMSO (6 mL). The mixture was stirred at 100° C. for 18 hr. After cooling to 25° C., the reaction mixture was diluted with DCM (40 mL). The organic layers were washed with H₂O (20 mL) and brine (20 mL), dried over Na₂SO₄, filtered and concentrated under reduced pressure to give a residue. The residue was purified by silica gel flash chromatography (IEluent of 0˜7% MeOH in CH₂Cl₂) to give tert-butyl N-[1-[[4-[2-(2-amino-3-pyridyl)-5-phenyl-imidazo[4,5-b]pyridin-3-yl]phenyl]methyl]-4-piperidyl]carbamate (100 mg, yield: 40%) as a yellow solid. MS: m/z=576.2 [M+H]⁺.

### Step 4: 3-(3-(4-((4-Aminopiperidin-1-yl)methyl)phenyl)-5-phenyl-3H-imidazo[4,5-b]pyridin-2-yl)pyridin-2-amine

To a solution of tert-butyl (1-(4-(2-(2-aminopyridin-3-yl)-5-phenyl-3H-imidazo[4,5-b]pyridin-3-yl)benzyl)piperidin-4-yl)carbamate (200 mg, 0.347 mmol) in HCl in 1,4-dioxane (4 M, 2 mL). The mixture was stirred at 25° C. for 2 hr. The reaction mixture was concentrated under reduced pressure to give a residue. The residue was purified by prep-HPLC (column: Waters xbridge 150*25 mm 10 um; mobile phase: [water (NH₄HCO₃)-ACN]; B %: 24%-54%, 8 min) to give 3-[3-[4-[(4-amino-1-piperidyl)methyl]phenyl]-5-phenyl-imidazo[4,5-b]pyridin-2-yl]pyridin-2-amine (Intermediate 11, 120 mg, yield: 72%) as a light-yellow solid. MS: m/z=476.2 [M+H]⁺. ¹H NMR (400 MHz, Chloroform-d) δ 8.12 (d, J=8.4 Hz, 1H), 8.05 (dd, J=5.2, 1.6 Hz, 1H), 8.01 (d, J=7.2 Hz, 2H), 7.79 (d, J=8.4 Hz, 1H), 7.50-7.35 (m, 7H), 7.09 (dd, J=7.6, 1.2 Hz, 1H), 6.61 (br s, 2H), 6.35 (dd, J=7.6, 4.8 Hz, 1H), 3.58 (s, 2H), 2.88 (br d, J=11.6 Hz, 2H), 2.75-2.65 (m, 1H), 2.09 (br t, J=11.6 Hz, 2H), 1.83 (br d, J=11.6 Hz, 2H), 1.49-1.38 (m, 2H).

### Intermediate 12: N-(3-(3-(4-((4-aminopiperidin-1-yl)methyl)phenyl)-2-(2-aminopyridin-3-yl)-3H-imidazo[4,5-b]pyridin-5-yl)phenyl)acetamide

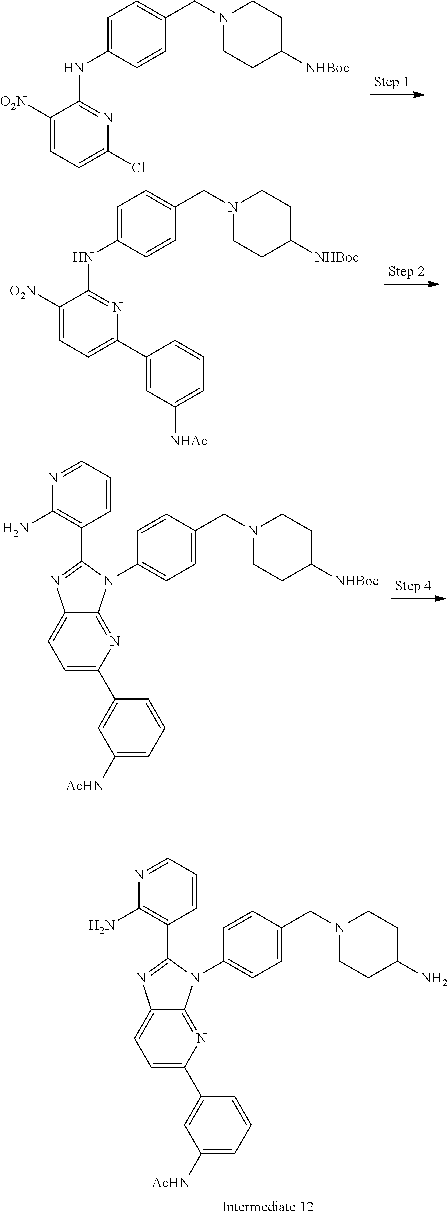

### Step 1: Tert-butyl (1-(4-((6-(3-acetamidophenyl)-3-nitropyridin-2-yl)amino)benzyl)piperidin-4-yl)carbamate

A mixture of tert-butyl N-[1-[[4-[(6-chloro-3-nitro-2-pyridyl)amino]phenyl]methyl]-4-piperidyl]carbamate (1.0 g, 2.16 mmol), (3-acetamidophenyl)boronic acid (773 mg, 4.32 mmol), Pd(dppf)Cl₂ (158 mg, 0.216 mmol) and K₂CO₃ (895 mg, 6.48 mmol) in H₂O (2 mL) and 1,4-dioxane (10 mL) was degassed and purged with N₂ three times, and then the mixture was stirred at 100° C. for 12 hr under N₂ atmosphere. The reaction mixture was diluted with H₂O (50 mL) and extracted with CH₂Cl₂ (50 mL×2). The combined organic layers were washed with brine (50 mL), dried over Na₂SO₄, filtered, and concentrated under reduced pressure to give a residue. The residue was purified by silica gel flash chromatography (Eluent of 0˜5% MeOH in CH₂Cl₂) to give tert-butyl N-[1-[[4-[[6-(3-acetamidophenyl)-3-nitro-2-pyridyl]amino]phenyl]methyl]-4-piperidyl]carbamate (1.04 g, yield: 86%) as an orange solid. MS: m/z=561.1 [M+H]⁺. ¹H NMR (400 MHz, Chloroform-d) δ 10.30 (s, 1H), 8.57 (d, J=8.8 Hz, 1H), 8.26 (br s, 1H), 7.79 (d, J=8.0 Hz, 1H), 7.72 (d, J=8.4 Hz, 2H), 7.63-7.57 (m, 1H), 7.47-7.35 (m, 4H), 7.28 (d, J=8.8 Hz, 1H), 4.44 (br s, 1H), 3.51 (s, 2H), 3.44-3.48 (m, 1H), 2.93-2.78 (m, 2H), 2.23 (s, 3H), 2.13 (br t, J=10.4 Hz, 2H), 1.93 (br d, J=11.2 Hz, 2H), 1.52-1.45 (m, 2H), 1.44 (s, 9H).

### Step 2: Tert-butyl (1-(4-((3-nitro-6-phenylpyridin-2-yl)amino)benzyl)piperidin-4-yl)carbamate

To a solution of tert-butyl N-[1-[[4-[[6-(3-acetamidophenyl)-3-nitro-2-pyridyl]amino]phenyl]methyl]-4-piperidyl]carbamate (950 mg, 1.69 mmol), 2-aminopyridine-3-carbaldehyde (228 mg, 1.9 mmol) and Na₂SO₄ (590 mg, 3.4 mmol) in DMSO (12 mL). The mixture was stirred at 100° C. for 18 hr. After cooling to 25° C., the reaction mixture was diluted with CH₂Cl₂ (50 mL). The organic layers were washed with H₂O (50 mL) and brine (50 mL), dried over Na₂SO₄, filtered, and concentrated under reduced pressure to give a residue. The residue was purified by silica gel flash chromatography (Eluent of 0˜˜7% MeOH in CH₂Cl₂) to give tert-butyl N-[1-[[4-[5-(3-acetamidophenyl)-2-(2-amino-3-pyridyl)imidazo[4,5-b]pyridin-3-yl]phenyl]methyl]-4-piperidyl]carbamate (536 mg, yield: 50%) as a yellow solid. MS: m/z=633.3 [M+H]⁺. ¹H NMR (400 MHz, Chloroform-d) δ 8.11 (d, J=8.4 Hz, 1H), 8.06 (dd, J=4.8, 2.0 Hz, 1H), 8.00 (br s, 1H), 7.78 (d, J=8.4 Hz, 1H), 7.73 (d, J=8.0 Hz, 1H), 7.66 (d, J=8.0 Hz, 1H), 7.49 (br s, 1H), 7.45 (d, J=8.4 Hz, 2H), 7.40-7.34 (m, 3H), 7.05 (dd, J=8.0, 1.6 Hz, 1H), 6.60 (br s, 2H), 6.33 (dd, J=7.6, 4.8 Hz, 1H), 4.47 (br s, 1H), 3.58 (s, 2H), 3.51-3.48 (m, 1H), 2.85 (br d, J=11.2 Hz, 2H), 2.18-2.16 (m, 2H), 2.14 (s, 3H), 1.94 (br d, J=10.8 Hz, 2H), 1.49-1.47 (m, 2H), 1.45 (s, 9H).

### Step 3: N-(3-(3-(4-((4-aminopiperidin-1-yl)methyl)phenyl)-2-(2-aminopyridin-3-yl)-3H-imidazo[4,5-b]pyridin-5-yl)phenyl)acetamide

A solution of tert-butyl N-[1-[[4-[5-(3-acetamidophenyl)-2-(2-amino-3-pyridyl)imidazo[4,5-b]pyridin-3-yl]phenyl]methyl]-4-piperidyl]carbamate (300 mg, 474 mmol) in HCl/1,4-dioxane (4 M, 2 mL) was stirred at 25° C. for 2 hr. The reaction mixture was added NaHCO₃ to adjust the pH abount 8 and extracted with DCM (15 mL×3). The combined organic layers were washed with brine (10 mL×2), dried by Na₂SO₄, filtered and concentrated under reduced pressure to give N-[3-[3-[4-[(4-amino-1-piperidyl)methyl]phenyl]-2-(2-amino-3-pyridyl)imidazo[4,5-b]pyridin-5-yl]phenyl]acetamide (Intermediate 12, 134 mg, yield: 53%) as a yellow solid. MS: m/z=533.2 [M+H]⁺. ¹H NMR (400 MHz, Chloroform-d) δ 8.13 (d, J=8.4 Hz, 1H), 8.07 (dd, J=4.8, 1.6 Hz, 1H), 8.02 (s, 1H), 7.81 (d, J=8.4 Hz, 1H), 7.76 (d, J=8.0 Hz, 1H), 7.69 (d, J=8.0 Hz, 1H), 7.48 (d, J=8.4 Hz, 2H), 7.42-7.37 (m, 4H), 7.08 (d, J=6.8 Hz, 1H), 6.61 (br s, 2H), 6.36 (dd, J=7.6, 4.8 Hz, 1H), 3.59 (s, 2H), 2.90 (br d, J=11.2 Hz, 2H), 2.77-2.67 (m, 1H), 2.20 (s, 3H), 2.12 (br t, J=11.2 Hz, 2H), 1.85 (br d, J=11.2 Hz, 2H), 1.49-1.41 (m, 2H).

### Intermediate 13: Methyl 4-[2-(2-amino-3-pyridyl)-5-phenyl-imidazo[4,5-b]pyridin-3-yl]benzoate

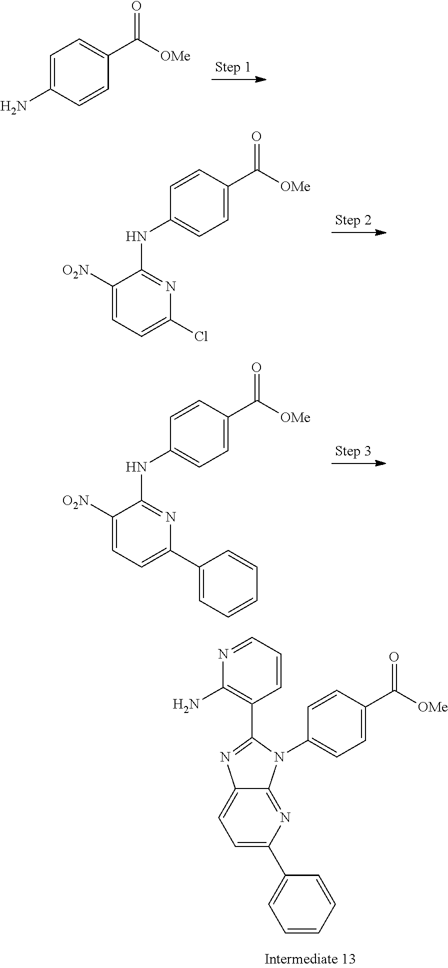

### Step 1: Methyl 4-((6-chloro-3-nitropyridin-2-yl)amino)benzoate

To a solution of methyl 4-aminobenzoate (5 g, 33.1 mmol) in DMSO (50 mL) was added 2,6-dichloro-3-nitro-pyridine (7.66 g, 39.7 mmol) and DIEA (12.82 g, 99.2 mmol). The mixture was stirred at 80° C. for 16 hr. After cooling to 20° C., the reaction mixture was poured into H₂O (100 mL) and extracted with CH₂Cl₂ (100 mL×3). The combined organic layers were washed with brine (100 mL×2), dried over anhydrous Na₂SO₄, filtered, and concentrated to dryness. The crude product was triturated with EtOAc at 25° C. for 30 min to give methyl 4-[(6-chloro-3-nitro-2-pyridyl)amino]benzoate (8 g, yield: 51%) as a yellow solid. MS: m/z=307.8 [M+H]⁺. ¹H NMR (400 MHz, Dimethylsulfoxide-d₆) δ 10.25 (s, 1H), 8.57 (d, J=8.4 Hz, 1H), 7.97 (d, J=8.8 Hz, 2H), 7.79 (d, J=8.8 Hz, 2H), 7.12 (d, J=8.4 Hz, 1H), 3.85 (s, 3H).

### Step 2: Methyl 4-((3-nitro-6-phenylpyridin-2-yl)amino)benzoate

To a solution of methyl 4-[(6-chloro-3-nitro-2-pyridyl)amino]benzoate (45 g, 146 mmol) and phenylboronic acid (21.4 g, 176 mmol) in 1,4-dioxane (500 mL) and H₂O (100 mL) were added Pd(dppf)Cl₂ (10.7 g, 14.6 mmol) and Cs₂CO₃ (143 g, 439 mmol). The mixture was degassed and purged with N₂ three times, and then the mixture was stirred at 80° C. for 16 hr under N₂ atmosphere. The reaction mixture was poured into H₂O (500 mL) and extracted with CH₂Cl₂ (500 mL×3). The combined organic layers were washed with brine (500 mL×3), dried over anhydrous Na₂SO₄, filtered, and concentrated to dryness. The crude product was triturated with EtOAc at 25° C. for 30 min to give methyl 4-[(3-nitro-6-phenyl-2-pyridyl)amino]benzoate (35.2 g, yield: 69%) as a red solid. MS: m/z=350.0 [M+H]⁺. ¹H NMR (400 MHz, Dimethylsulfoxide-d₆) δ 10.25 (s, 1H), 8.62 (d, J=8.8 Hz, 1H), 8.15-8.10 (m, 2H), 8.00 (d, J=8.4 Hz, 2H), 7.92 (d, J=8.4 Hz, 2H), 7.67 (d, 0.1=8.8 Hz, 1H), 7.59-7.54 (m, 3H), 3.86 (s, 3H)

### Step 3: Methyl 4-(2-(2-aminopyridin-3-yl)-5-phenyl-3H-imidazo[4,5-b]pyridin-3-yl)benzoate

To a solution of methyl 4-[(3-nitro-6-phenyl-2-pyridyl)amino]benzoate (15 g, 42.9 mmol) in DMSO (150 mL) was added 2-aminopyridine-3-carbaldehyde (6.29 g, 51.5 mmol) and Na₂S₂O₄ (15 g, 85.9 mmol). Then the reaction mixture was heated to 100° C. for 16 hr. After cooling to 25° C., the reaction mixture was diluted with H₂O (200 mL) and extracted with CH₂Cl₂ (200 ml×3), the combined organic layers were washed with brine brine (200 ml×2), dried over Na₂SO₄, filtered, and concentrated under reduced pressure to give a residue. The crude product was triturated with CH₂Cl₂ at 25° C. for 30 min to give methyl 4-[2-(2-amino-3-pyridyl)-5-phenyl-imidazo[4,5-b]pyridin-3-yl]benzoate (Intermediate 13, 12 g, yield. 66%) as a yellow solid. MS: m/z=422.0 [M+H]⁺. ¹H NMR (400 MHz, Dimethylsulfoxide-d₆) δ 8.29 (d, J=8.0 Hz, 1H), 8.12 (d, J=8.4 Hz, 2H), 8.07-8.00 (m, 4H), 7.67 (d, J=8.8 Hz, 2H), 7.49-7.44 (m, 2H), 7.42-7.38 (m, 1H), 7.23 (dd, J=7.6, 1.6 Hz, 1H), 6.89 (br s, 2H), 6.46 (dd, J=7.6, 4.8 Hz, 1H), 3.90 (s, 3H).

### Intermediate 14: 3-(3-(4-(Chloromethyl)phenyl)-5-phenyl-3H-imidazo[4,5-b]pyridin-2-yl)pyridin-2-amine

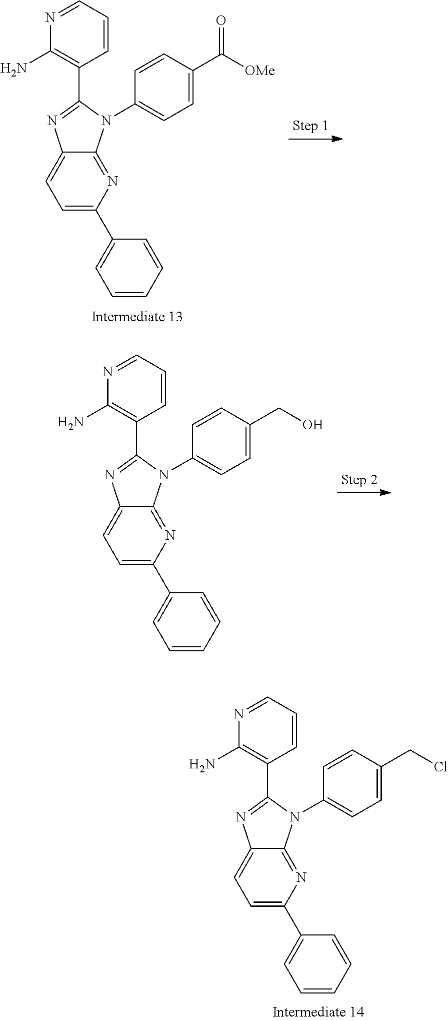 [SMILES: COC(=O)c1ccc(-n2c(-c3cccnc3N)nc3ccc(-c4ccccc4)nc32)cc1 ; Nc1ncccc1-c1nc2ccc(-c3ccccc3)nc2n1-c1ccc(CO)cc1 ; Nc1ncccc1-c1nc2ccc(-c3ccccc3)nc2n1-c1ccc(CCl)cc1]

### Step 1: (4-(2-(2-Aminopyridin-3-yl)-5-phenyl-3H-imidazo[4,5-b]pyridin-3-yl)phenyl)methanol

To a solution of Intermediate 13 (2.5 g, 5.9 mmol) in THF (25 mL) was added LiAlH₄ (450 mg, 11.9 mmol) at 0° C. After addition, the resulting mixture was stirred at 25° C. for 2 hr. After the reaction mixture was cooled to 0° C., the reaction mixture was quenched with H₂O (100 mL), followed by 15% aqueous NaOH (30 mL). Then the reaction mixture was filtered. The filter liquor was concentrated to dryness to give [4-[2-(2-amino-3-pyridyl)-5-phenyl-imidazo[4,5-b]pyridin-3-yl]phenyl]methanol (1.87 g, yield: 80%) as a yellow solid, which was directly used to the next step without further purification. MS: m/z=394.1 [M+H]⁺.

### Step 2: 3-(3-(4-(Chloromethyl)phenyl)-5-phenyl-3H-imidazo[4,5-b]pyridin-2-yl)pyridin-2-amine

To a solution of [4-[2-(2-amino-3-pyridyl)-5-phenyl-imidazo[4,5-b]pyridin-3-yl]phenyl]methanol (2.3 g, 5.9 mmol) in CH₂Cl₂ (25 mL) was added SOCl₂ (2.1 g, 17.5 mmol). The mixture was stirred at 40° C. for 1 hr. The reaction mixture was filtered. The filter liquor was concentrated to dryness to give 3-(3-(4-(chloromethyl)phenyl)-5-phenyl-3H-imidazo[4,5-b]pyridin-2-yl)pyridin-2-amine (Intermediate 14, 1.71 g, yield: 71%) as a yellow solid. MS: m/z=412.0 [M+H]⁺. ¹H NMR (400 MHz, Dimethylsulfoxide-d₆) δ 8.29 (d, J=8.4 Hz, 1H), 8.12 (d, J=8.4 Hz, 2H), 8.07-8.00 (m, 4H), 7.67 (d, J=8.4 Hz, 2H), 7.50-7.44 (m, 2H), 7.42-7.37 (m, 1H), 7.24 (d, J=7.2 Hz, 1H), 6.88 (br s, 2H), 6.46 (dd, J=4.8, 7.6 Hz, 1H), 3.90 (s, 2H).

### Intermediate 15: 3-(3-(4-(Chloromethyl)phenyl)-3H-imidazo[4,5-b]pyridin-2-yl)pyridin-2-amine

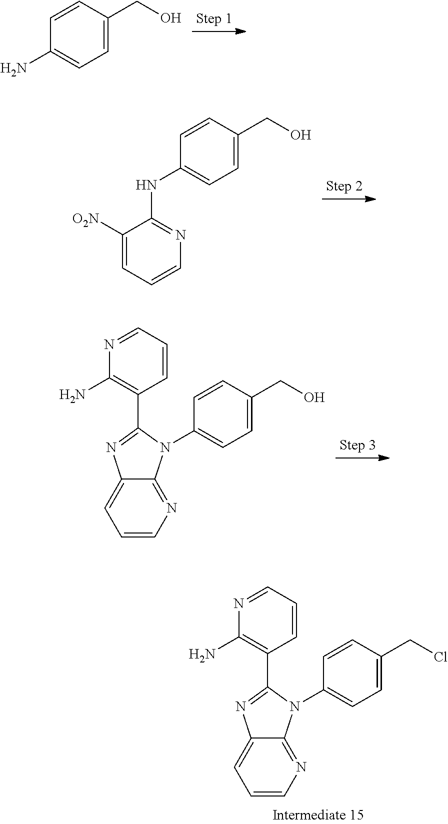

### Step 1: (4-((3-Nitropyridin-2-yl)amino)phenyl)methanol

To a solution of (4-aminophenyl)methanol (10 g, 81.2 mmol) in 1,4-dioxane (150 mL) was added DIEA (31.5 g, 244 mmol) and 2-chloro-3-nitro-pyridine (15.5 g, 97.4 mmol). The mixture was stirred at 100° C. for 16 hr. The reaction mixture was concentrated under reduced pressure to remove solvent. The residue was diluted with H₂O (200 mL) and extracted with EtOAc (200 mL×2). The combined organic layers were washed with brine (500 mL), dried over Na₂SO₄, filtered and concentrated under reduced pressure to give a residue. The crude product was purified by flash chromatography on silica gel (Eluent of 0˜82% EtOAc in petroleum ether) to give (4-((3-nitropyridin-2-yl)amino)phenyl)methanol (15.3 g, yield: 77%) as a yellow solid. MS: m/z=245.8 [M+H]⁺. ¹H NMR (400 MHz, Chloroform-d) δ 10.12 (s, 1H), 8.53 (dd, J=8.4, 2.0 Hz, 1H), 8.48 (dd, J=4.4, 1.6 Hz, 1H), 7.64 (d, J=8.0 Hz, 2H), 7.40 (d, J=8.4 Hz, 2H), 6.84 (dd, J=8.4, 4.8 Hz, 1H), 4.70 (s, 2H).

### Step 2: (4-(2-(2-Aminopyridin-3-yl)-3H-imidazo[4,5-b]pyridin-3-yl)phenyl)methanol

To a solution of 4-((3-nitropyridin-2-yl)amino)phenyl)methanol (10 g, 40.8 mmol) in DMSO (500 mL) was added Na₂S₂O₄ (21.3 g, 122 mmol) and 2-aminopyridine-3-carbaldehyde (5.98 g, 48.9 mmol). The mixture was stirred at 100° C. for 16 hr. The reaction mixture was quenched by H₂O (500 mL) at 20° C. and extracted with EtOAc (500 mL×2). The combined organic layers were washed with brine (500 mL), dried over Na₂SO₄, filtered, and concentrated under reduced pressure to give a residue. The crude product was purified by flash chromatography on silica gel (Eluent of 0˜100% EtOAc in petroleum ether) to give (4-(2-(2-aminopyridin-3-yl)-3H-imidazo[4,5-b]pyridin-3-yl)phenyl)methanol (3.1 g, yield: 24%) as a yellow solid. MS: m/z=318.3 [M+H]⁺. ¹H NMR (400 MHz, Chloroform-d) δ 8.39-8.32 (m, 1H), 8.08 (dd, J=8.0, 1.2 Hz, 1H), 8.02 (dd, J=4.8, 1.6 Hz, 1H), 7.52 (d, J=8.4 Hz, 2H), 7.35 (d, J=8.0 Hz, 2H), 7.30-7.27 (m, 1H), 7.12 (dd, J=7.6, 1.6 Hz, 1H), 6.65 (br s, 2H), 6.35 (dd, J=7.6, 4.8 Hz, 1H), 4.76 (s, 2H).

### Step 3: 3-(3-(4-(Chloromethyl)phenyl)-3H-imidazo[4,5-b]pyridin-2-yl)pyridin-2-amine

To a solution of [4-[2-(2-amino-3-pyridyl)imidazo[4,5-b]pyridin-3-yl]phenyl]methanol (1.0 g, 3.15 mmol) in CH₂Cl₂ (10 mL) was added SOCl₂ (3.3 g, 27.6 mmol). The mixture was stirred at 40° C. for 1 hr. The reaction mixture was concentrated under reduced pressure to remove solvent to give 3-(3-(4-(chloromethyl)phenyl)-3H-imidazo[4,5-b]pyridin-2-yl)pyridin-2-amine (Intermediate 15, 1.2 g HCl salt) as a gray solid, which was used to the next step without further purification. MS: m/z=336.0 [M+H]⁺. ¹H NMR (400 MHz, Chloroform-d) δ 9.35 (br s, 1H), 8.55 (d, J=8.0 Hz, 1H), 8.28 (d, J=7.6 Hz, 1H), 7.91-7.83 (m, 1H), 7.68 (d, J=8.0 Hz, 2H), 7.64-7.59 (m, 1H), 7.54-7.50 (m, 1H), 7.46 (d, J=8.0 Hz, 2H), 7.30-7.27 (m, 2H), 6.70-6.60 (m, 1H), 4.70 (s, 2H).

### Intermediate 16: 6-(Piperidin-4-yloxy) nicotinonitrile

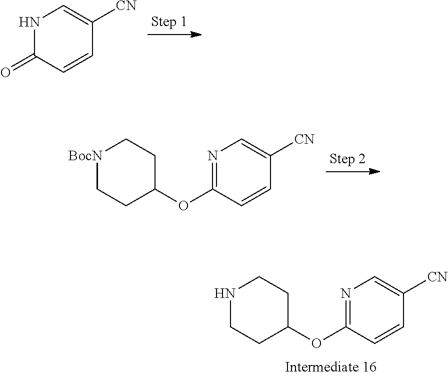 [SMILES: N#Cc1ccc(=O)[nH]c1 ; CC(C)(C)OC(=O)N1CCC(Oc2ccc(C#N)cn2)CC1 ; N#Cc1ccc(OC2CCNCC2)nc1]

### Step 1: Tert-butyl 4-((5-cyanopyridin-2-yl)oxy)piperidine-1-carboxylate

To a solution of 6-oxo-1H-pyridine-3-carbonitrile (1.0 g, 8.33 mmol) in THF (30 mL) was added PPh₃ (4.4 g, 16.7 mmol), then the mixture was degassed and purged with N₂ three times, DEAD (2.9 g, 16.7 mmol) and tert-butyl 4-hydroxypiperidine-1-carboxylate (1.8 g, 9.16 mmol) was added dropwise at 0° C. The resulting mixture was stirred at 25° C. for 16 hr. The mixture was 3 diluted with H₂O (50 mL) and extracted with EtOAc (50 mL×2). The combined organic layers were washed with 50 mL brine, dried over Na₂SO₄, filtered and concentrated under reduced pressure to give a residue. The crude product was purified by flash chromatography on silica gel (Eluent of 0˜25% EtOAc in petroleum ether) to give tert-butyl 4-((5-cyanopyridin-2-yl)oxy)piperidine-1-carboxylate (2.5 g, yield: 99%) as a pink solid. ¹H NMR (400 MHz, Chloroform-d) δ 8.44 (d, J=2.4 Hz, 1H), 7.77 (dd, J=8.8, 2.4 Hz, 1H), 6.78 (d, 1=8.4 Hz, 1H), 5.30-5.26 (m, 1H), 3.79-3.74 (m, 2H), 3.30-3.25 (m, 2H), 1.99-1.95 (m, 2H), 1.74-1.70 (m, 2H), 1.46 (s, 9H).

### Step 2: 6-(Piperidin-4-yloxy) nicotinonitrile

To a solution of tert-butyl 4-[(5-cyano-2-pyridyl)oxy]piperidine-1-carboxylate (200 mg, 659 μmol) in HCl in 1,4-dioxane (4 M, 2 mL). The mixture was stirred at 25° C. for 1 hr. The reaction mixture was concentrated under reduced pressure to remove solvent to give 6-(piperidin-4-yloxy) nicotinonitrile (Intermediate 16, 150 mg, HCl salt) as a pink solid, which was directly used to the next step without further purification. ¹H NMR (400 MHz, Chloroform-d) δ 9.64 (br s, 2H), 8.45 (d, J=1.6 Hz, 1H), 7.82 (dd, J=8.4, 1.6 Hz, 1H), 6.83 (d, =8.4 Hz, 1H), 5.43 (br s, 1H), 4.28-4.14 (m, 1H), 3.44-3.24 (m, 4H), 2.40-2.16 (m, 4H).

### Intermediate 17: 6-(Piperidin-4-yloxy)picolinonitrile

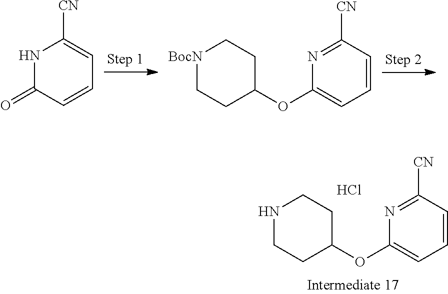 [SMILES: N#Cc1cccc(=O)[nH]1 ; CC(C)(C)OC(=O)N1CCC(Oc2cccc(C#N)n2)CC1 ; N#Cc1cccc(OC2CCNCC2)n1 ; Cl]

### Step 1: Tert-butyl 4-((6-cyanopyridin-2-yl)oxy)piperidine-1-carboxylate

To a solution of 6-oxo-1,6-dihydropyridine-2-carbonitrile (500 mg, 4.2 mmol) and tert-butyl 4-hydroxypiperidine-1-carboxylate (922 mg, 4.6 mmol) in THF (15 mL) was added PPh₃ (2.2 g, 8.3 mmol) was degassed and purged with N₂ three times, and then DEAD (1.5 g, 8.3 mmol) was added dropwise at 0° C. The resulting mixture was stirred at 25° C. for 16 hr. The mixture was diluted with H₂O (20 mL) and extracted with EtOAc (30 mL×2). The combined organic layers were washed with 20 mL brine, dried over Na₂SO₄, filtered, and concentrated under reduced pressure to give a residue. The crude product was purified by flash chromatography on silica gel (Eluent of 0˜25% EtOAc in petroleum ether), tert-butyl 4-((6-cyanopyridin-2-yl)oxy)piperidine-1-carboxylate (1.2 g, yield: 94%) was obtained as a white solid. ¹H NMR (400 MHz, Chloroform-d) δ 7.63 (dd, J=8.4, 7.2 Hz, 1H), 7.24 (d, J=8.0 Hz, 1H), 6.90 (d, J=8.4 Hz, 1H), 5.27-5.17 (m, 1H), 3.79-3.70 (m, 2H), 3.30-3.21 (m, 2H), 1.99-1.91 (m, 2H), 1.72-1.65 (m, 2H), 1.44 (s, 9H).

### Step 2: 6-4-Piperidin-4-yloxy)picolinonitrile

To a solution of tert-butyl 4-((6-cyanopyridin-2-yl)oxy)piperidine-1-carboxylate (500 mg, 1.7 mmol) in HCl in 1,4-dioxane (4 M, 5 mL). The mixture was stirred at 25° C. for 1 hr. The reaction mixture was concentrated under reduced pressure to give 6-(piperidin-4-yloxy)picolinonitrile (Intermediate 17, 330 mg, HCl salt, yield: 81%) as a white solid, which was used to the next step without further purification.

### Intermediate 18: 2-(Piperidin-4-yloxy)isonicotinonitrile

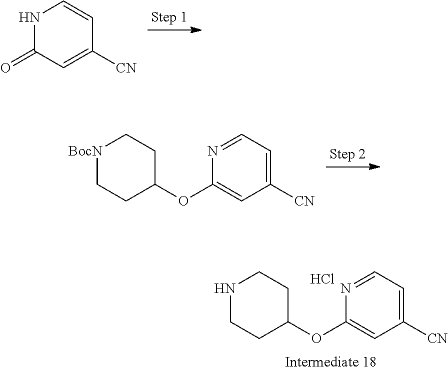 [SMILES: N#Cc1cc[nH]c(=O)c1 ; CC(C)(C)OC(=O)N1CCC(Oc2cc(C#N)ccn2)CC1 ; N#Cc1ccnc(OC2CCNCC2)c1 ; Cl]

### Step 1: Tert-butyl 4-((4-cyanopyridin-2-yl)oxy)piperidine-1-carboxylate

Following the general procedure of Intermediate 17, the reaction of 2-oxo-1,2-dihydropyridine-4-carbonitrile (1.0 g, 8.3 mmol) with tert-butyl 4-hydroxypiperidine-1-carboxylate (1.8 g, 9.2 mmol) was carried out. After purified flash chromatography on silica gel (Eluent of 0˜20% EtOAc in petroleum ether), tert-butyl 4-((4-cyanopyridin-2-yl)oxy)piperidine-1-carboxylate (2.1 g, yield: 83%) was obtained as a pink solid. ¹H NMR (400 MHz, Chloroform-d) δ 8.25 (d, J=5.2 Hz, 1H), 7.04 (dd, 1=5.2, 1.2 Hz, 1H), 6.96 (s, 1H), 5.26-5.20 (m, 1H), 3.80-3.72 (m, 2H), 3.31-3.24 (m, 2H), 1.99-1.93 (m, 2H), 1.75-1.68 (m, 2H), 1.46 (s, 9H).

### Step 2: 2-(Piperidin-4-yloxy)isonicotinonitrile

Following the general procedure of Intermediate 17, the reaction of tert-butyl 4-((4-cyanopyridin-2-yl)oxy)piperidine-1-carboxylate (500 mg, 1.7 mmol) in HCl in 1,4-dioxane (5 mL) was carried out, 2-(piperidin-4-yloxy)isonicotinonitrile (Intermediate 18, 330 mg, HCl salt, yield: 81%) was obtained as a pink solid, which was used to the next step without further purification.

### Intermediate 19: 2-(Piperidin-4-yloxy)nicotinonitrile

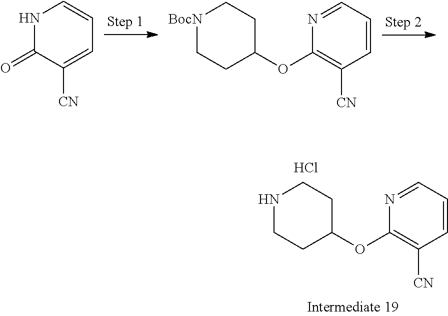 [SMILES: N#Cc1ccc[nH]c1=O ; CC(C)(C)OC(=O)N1CCC(Oc2ncccc2C#N)CC1 ; Cl ; N#Cc1cccnc1OC1CCNCC1]

### Step 1: Tert-butyl 4-((3-cyanopyridin-2-yl)oxy)piperidine-1-carboxylate

Following the general procedure of Intermediate 17, the reaction of 2-oxo-1,2-dihydropyridine-3-carbonitrile (1.0 g, 8.3 mmol) with tert-butyl 4-hydroxypiperidine-1-carboxylate (1.8 g, 9.2 mmol) was carried out. After purified flash chromatography on silica gel (Eluent of 0˜20% EtOAc in petroleum ether), tert-butyl 4-((3-cyanopyridin-2-yl)oxy)piperidine-1-carboxylate (2.3 g, yield: 91%) was obtained as a pink solid. ¹H NMR (400 MHz, Chloroform-d) δ 8.31 (dd, J=5.2, 2.0 Hz, 1H), 7.87 (dd, J=7.6, 2.0 Hz, 1H), 6.95 (dd, J=7.6, 5.2 Hz, 1H), 5.39-5.32 (m, 1H), 3.76-3.69 (m, 2H), 3.42-3.35 m, 2H), 2.00-1.93 (m, 2H), 1.85-1.78 (m, 2H), 1.46 (s, 9H).

### Step 2: 2-(Piperidin-4-yloxy)nicotinonitrile

Following the general procedure of Intermediate 17, the reaction of tert-butyl 4-((3-cyanopyridin-2-yl)oxy)piperidine-1-carboxylate (500 mg, 1.7 mmol) in HCl in 1,4-dioxane (4 M, 5 mL) was carried out, 2-(piperidin-4-yloxy)nicotinonitrile (Intermediate 19, 330 mg, HCl salt, yield: 81%) was obtained as a pink solid, which was used to the next step without further purification.

### Intermediate 20: 3-(3-(4-(Chloromethyl)phenyl)-6-phenyl-3H-imidazo[4,5-b]pyridin-2-yl)pyridin-2-amine

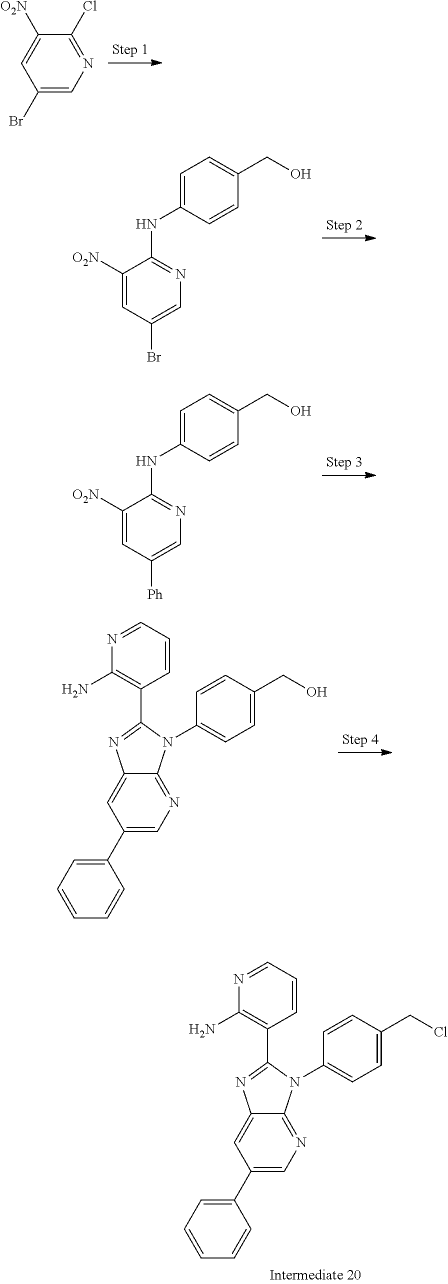

### Step 1: (4-((5-Bromo-3-nitropyridin-2-yl)amino)phenyl)methanol

To a solution of 5-bromo-2-chloro-3-nitro-pyridine (10 g, 42 mmol) and (4-aminophenyl)methanol (5.2 g, 42 mmol) in DMSO (100 mL) was added DIEA (16.3 g, 126 mmol). The mixture was stirred at 25° C. for 16 hr. Then the reaction mixture was poured into H₂O (200 mL) and extracted with EtOAc (300 mL×2). The combined organic layers were washed with brine (200 mL×2), dried over anhydrous Na₂SO₄, filtered, and concentrated to dryness. The residue was purified by silica gel flash chromatography (Eluent of 10˜50% EtOAc in petroleum ether) to give (4-((5-bromo-3-nitropyridin-2-yl)amino)phenyl)methanol (6.28 g, yield: 46%) as a red solid. MS: m/z=324.0, 325.0 [M+H]+. ¹H NMR (400 MHz, Chloroform-d-) δ 10.05 (br s, 1H), 8.65 (d, J=2.4 Hz, 1H), 8.50 (d, J=2.4 Hz, 1H), 7.59 (d, J=8.4 Hz, 2H), 7.40 (d, J=8.4 Hz, 2H), 4.71 (s, 2H).

### Step 2: (4-((3-Nitro-5-phenylpyridin-2-yl)amino)phenyl)methanol

A mixture of [4-[(5-bromo-3-nitro-2-pyridyl)amino]phenyl]methanol (5.4 g, 16.7 mmol), phenylboronic acid (6.1 g, 50 mmol), K₂CO₃ (4.62 g, 33.44 mmol), Pd(dppf)Cl₂ (1.22 g, 1.67 mmol) in 1,4-dioxane (60 mL) and H₂O (12 mL) was degassed and purged with N₂ three times, and then the mixture was stirred at 100° C. for 8 hr under N₂ atmosphere. The reaction mixture was poured into H₂O (100 mL) and extracted with CH₂Cl₂ (100 mL×3). The combined organic layers were washed with brine (100 mL×2), dried over anhydrous Na₂SO₄, filtered, and concentrated to dryness. The residue was purified by silica gel flash chromatography (Eluent of 0˜50% EtOAc in petroleum ether) to give [4-[(3-nitro-5-phenyl-2-pyridyl)amino]phenyl]methanol (4.6 g, yield: 86%) as a red solid. MS: m/z=322.1 [M+H]⁺. ¹H NMR (400 MHz, Chloroform-d) δ 10.15 (s, 1H), 8.78-8.72 (m, 2H), 8.25 (d, J=8.0 Hz 1H), 7.70-7.65 (m, 2H), 7.59-7.56 (m, 2H), 7.51-7.49 (m, 2H), 7.44-7.41 (m, 2H), 4.72 (s, 2H).

### Step 3: (4-(2-(2-Aminopyridin-3-yl)-6-phenyl-3H-imidazo[4,5-b]pyridin-3-yl)phenyl)methanol

To a solution of [4-[(3-nitro-5-phenyl-2-pyridyl)amino]phenyl]methanol (15 g, 46.7 mmol) in DMSO (600 mL) was added Na₂S₂O₄ (16 g, 93 mmol) and 2-aminopyridine-3-carbaldehyde (6.8 g, 56 mmol). The mixture was stirred at 100° C. for 12 hr. After cooling to 25° C., the reaction mixture was diluted with H₂O (200 mL) and extracted with CH₂Cl₂ (200 ml×3), the combined organic layers were washed with brine (200 ml×2), dried over Na₂SO₄, filtered, and concentrated under reduced pressure to give a residue. The residue was purified by silica gel flash chromatography (Eluent of 0˜100% EtOAc in petroleum ether) to give [4-[2-(2-amino-3-pyridyl)-6-phenyl-imidazo[4,5-b]pyridin-3-yl]phenyl]methanol (5.15 g crude, yield: 28%) as a yellow solid. MS: m/z=394.0 [M+H]⁺.

### Step 4: 3-(3-(4-(Chloromethyl)phenyl)-6-phenyl-3H-imidazo[4,5-b]pyridin-2-yl)pyridin-2-amine

To a solution of [4-[2-(2-amino-3-pyridyl)-6-phenyl-imidazo[4,5-b]pyridin-3-yl]phenyl]methanol (500 mg, 1.3 mmol) in CH₂Cl₂ (10 mL) was added SOCl₂ (771 mg, 6.5 mmol). The mixture was stirred at 40° C. for 2 hr. The reaction mixture was concentrated under reduced pressure to give a residue. The residue was purified by silica gel flash chromatography (Eluent of 0˜100% EtOAc in petroleum ether) to give 3-[3-[4-(chloromethyl)phenyl]-6-phenyl-imidazo[4,5-b]pyridin-2-yl]pyridin-2-amine (Intermediate 20, 267 mg, yield: 51%) as a yellow solid. MS: m/z=411.8 [M+H]⁺. ¹H NMR (400 MHz, Dimethylsulfoxide-d₆) δ 8.63 (d, J=2.0 Hz, 1H), 8.47 (d, J=2.0 Hz, 1H), 8.01 (dd, J=8.8, 3.2 Hz, 1H), 7.79 (d, J=7.2 Hz, 2H), 7.61 (d, J=8.4 Hz, 2H), 7.55-7.47 (m, 4H), 7.45-7.40 (m, 1H), 7.25 (dd, J=7.6, 1.8 Hz, 1H), 6.96 (br s, 2H), 6.44 (dd, J=7.6, 4.8 Hz, 1H), 4.86 (s, 2H).

### Intermediate 21: 6-Chloropyridazine-4-carbonitrile

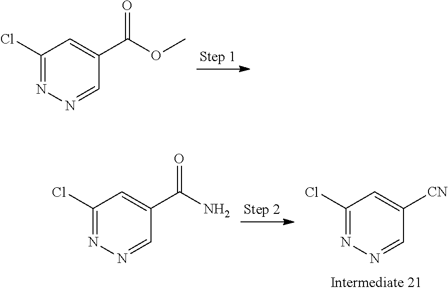

### Step 1: 6-Chloropyridazine-4-carboxamide

A solution of methyl 6-chloropyridazine-4-carboxylate (9.0 g, 52 mmol) and ammonia in MeOH (7 M, 90 mL) was stirred at 80° C. for 3 hr. The residue was purified by silica gel flash chromatography (Eluent of 0˜98% EtOAc in petroleum ether), 6-chloropyridazine-4-carboxamide (6.4 g, yield: 78%) was obtained as a yellow solid. ¹H NMR (400 MHz, Dimethylsulfoxide-d₆) δ 9.52 (s, 1H), 8.48 (br s, 1H), 8.21 (s, 1H), 8.09 (br s, 1H).

### Step 2: 6-Chloropyridazine-4-carbonitrile

To a solution of 6-chloropyridazine-4-carboxamide (3.0 g, 19.0 mmol) in Py (30 mL) was added POCl₃ (14.6 g, 95.0 mmol). The mixture was stirred at 25° C. for 12 hr. The residue was purified by column chromatography (0˜30% EtOAc in petroleum ether), 6-chloropyridazine-4-carbonitrile (Intermediate 21, 1.48 g, yield: 56%) was obtained as a yellow solid. MS: m/z=140.0 [M+H]⁺. ¹H NMR (400 MHz, Dimethylsulfoxide-d₆) δ 9.67 (s, 1H), 8.64 (s, 1H).

### Intermediate 22: 5-(Piperidin-4-ylamino)nicotinonitrile

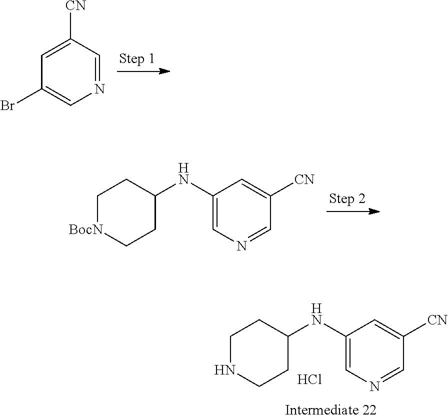 [SMILES: N#Cc1cncc(Br)c1 ; CC(C)(C)OC(=O)N1CCC(Nc2cncc(C#N)c2)CC1 ; N#Cc1cncc(NC2CCNCC2)c1 ; Cl]

### Step 1: Tert-butyl 4-((5-cyanopyridin-3-yl)amino)piperidine-1-carboxylate

A mixture of tert-butyl 4-aminopiperidine-1-carboxylate (1.3 g, 6.6 mmol), 5-bromonicotinonitrile (1.0 g, 5.5 mmol), BINAP (170 mg, 273 μmol), Pd(OAc)₂ (25 mg, 109 mmol) and Cs₂CO₃ (3.56 g, 10.9 mmol) in 1,4-dioxane (20 mL) was degassed and purged with N₂ three times, and then the mixture was stirred at 100° C. for 16 hr under N₂ atmosphere. The mixture was diluted with H₂O (50 mL) and extracted with EtOAc (50 mL/2). The combined organic layers were washed with 50 mL brine, dried over Na₂SO₄, filtered, and concentrated under reduced pressure to give a residue. The crude product was purified by flash chromatography on silica gel (Eluent of 0˜30% EtOAc in petroleum ether), tert-butyl 4-((5-cyanopyridin-3-yl)amino)piperidine-1-carboxylate (1.2 g, yield: 72%) was obtained as a light yellow solid. ¹H NMR (400 MHz, Chloroform-d) δ 8.16 (s, 2H), 7.06-6.97 (m, 1H), 4.17-3.98 (m, 3H), 3.41 (br s, 1H), 2.99-2.87 (m, 2H), 2.05-1.97 (m, 2H), 1.46 (s, 9H), 1.43-1.32 (m, 2H).

### Step 2: 5-(Piperidin-4-ylamino)nicotinonitrile

To a solution of tert-butyl 4-((5-cyanopyridin-3-yl)amino)piperidine-1-carboxylate (150 mg, 496 μmol) in HCl in 1,4-dioxane (4 M, 2 mL). The mixture was stirred at 25° C. for 1 hr. The reaction mixture was concentrated under reduced pressure to give 5-(piperidin-4-ylamino)nicotinonitrile (110 mg, HCl salt, yield: 93%) as a light-yellow solid, which was used to the next step without further purification.

### Intermediate 23: 3-Cyanopyridazine-4-carboxylic acid

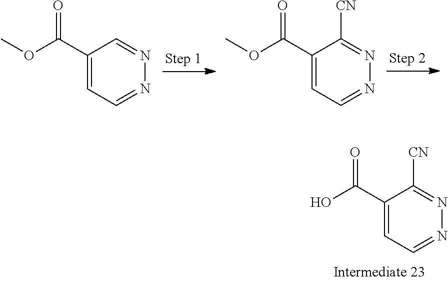

### Step 1: Methyl 3-cyanopyridazine-4-carboxylate

To a solution of methyl pyridazine-4-carboxylate (600 mg, 4.34 mmol) in CHCl₃ (25 mL) was added Tf₂O (1.47 g, 5.21 mmol). The mixture was stirred at 25° C. for 1 hr. And then TMSCN (2.15 g, 21.7 mmol) was added dropwise at 25° C. The reaction mixture was stirred at 60° C. for 3 hr. And then 4-methylmorpholine (571 mg, 5.65 mmol) was added dropwise at 60° C. The mixture was stirred at 60° C. for another 12 hr. The reaction mixture was quenched with NaHCO₃ (aq) (10 mL) at 25° C. and extracted with CH₂Cl₂ (50 mL×2). The combined organic layers were washed with brine (50 mL), dried over Na₂SO₄, filtered, and concentrated under reduced pressure to give a residue. The residue was purified by silica gel flash chromatography (Eluent of 0˜40% EtOAc in petroleum ether), methyl 3-cyanopyridazine-4-carboxylate (400 mg, yield: 56%) was obtained as a brown solid. ¹H NMR (400 MHz, Dimethylsulfoxide-d₆) 69.71 (d, J=5.6 Hz, 1H), 8.31 (d, J=5.6 Hz, 1H), 3.98 (s, 3H).

### Step 2: 3-Cyanopyridazine-4-carboxylic acid

To a solution of methyl 3-cyanopyridazine-4-carboxylate (400 mg, 2.45 mmol) in THF (5 mL) was added the mixture of LiOH·H₂O (205 mg, 4.90 mmol) in H₂O (5 mL) at 0° C. The reaction mixture was stirred at 0° C. for 0.5 hr. The reaction mixture was then adjusted to pH=5 by aq. HCl (1 N). The resulting mixture was extracted with CH₂Cl₂ (20 mL×3). The combined organic layers were dried over anhydrous Na₂SO₄, filtered, and concentrated to give a residue 3-cyanopyridazine-4-carboxylic acid (Intermediate 23, 350 mg, yield: 96%) as a brown solid. ¹H NMR (400 MHz, Dimethylsulfoxide-d₆) δ 9.68 (d, J=5.6 Hz, 1H), 8.26 (d, J=5.6 Hz, 1H).

### Intermediate 24: 5-Cyanopyrimidine-4-carboxylic acid

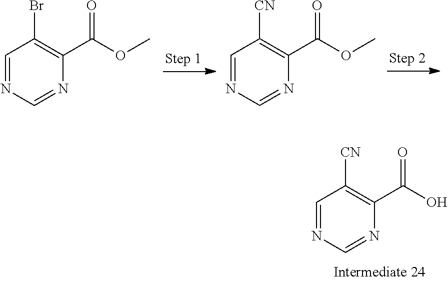

### Step 1: Methyl 5-cyanopyrimidine-4-carboxylate

To a solution of methyl 5-bromopyrimidine-4-carboxylate (400 mg, 1.84 mmol) and Zn(CN)₂ (216 mg, 1.84 mmol) in DMF (10 mL) was added DPPF (102 mg, 184 μmol) and Pd₂(dba)₃ (84 mg, 92.2 μmol) at 25° C. After addition the reaction mixture was stirred at 90° C. under N₂ atmosphere for 12 hr. The reaction mixture was poured into H₂O (50 mL). The resulting mixture was extracted with EtOAc (30 mL×2). The combined organic phase was washed with water (50 mL), dried over anhydrous Na₂SO₄, filtered and concentrated. The residue was purified by silica gel flash chromatography (Eluent of 0˜40% EtOAc in petroleum ether), methyl 5-cyanopyrimidine-4-carboxylate (130 mg, yield: 42%) was obtained as a yellow solid. ¹H NMR (400 MHz, Dimethylsulfoxide-d₆) δ 9.58 (s, 1H), 9.54 (s, 1H), 3.98 (s, 3H).

### Step 2: 5-Cyanopyrimidine-4-carboxylic acid

To a solution of methyl 5-cyanopyrimidine-4-carboxylate (400 mg, 2.45 mmol) in THF (4 mL) was added the mixture of LiOH·H₂O (205 mg, 4.90 mmol) in H₂O (4 mL) at 0° C. The reaction mixture was stirred at 0° C. for 0.5 hr. The reaction mixture was then adjusted to pH=5 by aq. HCl (1 N). The resulting mixture was extracted with CH₂Cl₂ (20 mL×3). The combined organic phase was dried over anhydrous Na₂SO₄, filtered, and concentrated to give a residue 5-cyanopyrimidine-4-carboxylic acid (Intermediate 24, 350 mg, yield: 96%) as a brown solid. ¹H NMR (400 MHz, Dimethylsulfoxide-d₆) δ 9.22 (s, 1H), 9.08 (s, 1H).

### Intermediate 25: 2-Carbamoylbenzo[d]thiazole-5-carboxylic acid

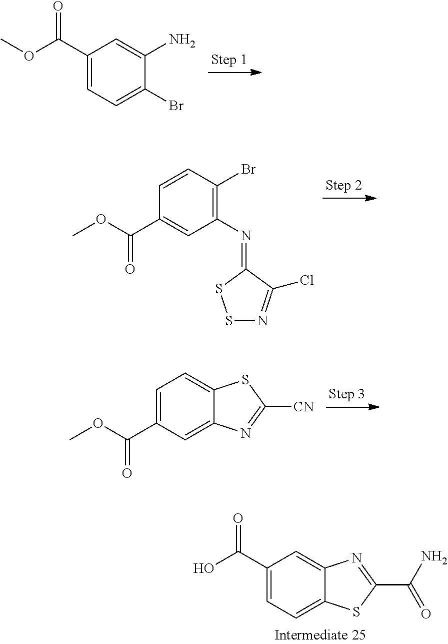 [SMILES: COC(=O)c1ccc(Br)c(N)c1 ; COC(=O)c1ccc(Br)c(/N=c2\ssnc2Cl)c1 ; COC(=O)c1ccc2sc(C#N)nc2c1 ; NC(=O)c1nc2cc(C(=O)O)ccc2s1]

### Step 1: Methyl (Z)-4-bromo-3-((4-chloro-5H-1,2,3-dithiazol-5-ylidene)amino)benzoate

To a solution of methyl 3-amino-4-bromo-benzoate (5.0 g, 22 mmol) in 50 mL DCM was added 4,5-dichlorodithiazol-2-ium;chloride (5.9 g, 28 mmol). The mixture was stirred at 25° C. for 3 hr. Then pyridine (3.4 g, 43 mmol) was added to the mixture, the resulting mixture was stirred at 25° C. for 2 hr. The mixture was concentrated under reduced pressure. The residue was purified by column chromatography (Eluent of 0˜10% EtOAc in petroleum ether), methyl methyl (Z)-4-bromo-3-((4-chloro-5H-1,2,3-dithiazol-5-ylidene)amino)benzoate (2.27 g, yield: 27%) was obtained as a yellow solid. MS: m/z=366.6 [M+H]+. ¹H NMR (400 MHz, Chloroform-d) δ 7.80 (d, J=1.6 Hz, 1H), 7.76 (s, 1H), 7.76 (d, 1=2.0 Hz, 1H), 3.92-3.92 (m, 3H).

### Step 2: (4-((3-Nitro-5-phenylpyridin-2-yl)amino)phenyl)methanol

Methyl 4-bromo-3-[(Z)-(4-chlorodithiazol-5-ylidene)amino]benzoate (1.13 g, 3.0 mmol) and CuI (588 mg, 3.0 mmol) were taken up into a microwave tube in pyridine (20 mL). The sealed tube was heated at 115° C. for 0.5 hr under microwave. The reaction mixture was diluted with EtOAc (50 mL) and quenched with Na₂SO₃ (50 mL) at 25° C. The combined organic layers were washed with brine (50 mL), dried over, filtered, and concentrated under reduced pressure to give a residue. The residue was purified by column chromatography (Eluent of 0˜5% EtOAc in petroleum ether), (4-((3-nitro-5-phenylpyridin-2-yl)amino)phenyl)methanol (1.0 g, yield: 70%) was obtained as a yellow solid. MS: m/z=218.9 [M+H]⁺. ¹H NMR (400 MHz, Chloroform-d) 68.89 (d, J=2.0 Hz, 1H), 8.33-8.26 (m, 1H), 8.05 (d, J=8.4 Hz, 1H), 4.01 (s, 3H).

### Step 3: 2-Carbamoylbenzo[d]thiazole-5-carboxylic acid

To a solution of methyl 2-cyano-1,3-benzothiazole-5-carboxylate (200 mg, 916 μmol) in THF (2 mL) was added LiOH·H₂O in H₂O (1 M, 1.37 mL). The mixture was stirred at 0° C. for 3 hr. The mixture was concentrated under pressure at 20° C., 2-Carbamoylbenzo[d]thiazole-5-carboxylic acid (Intermediate 25, 200 mg, used directly) was obtained as a yellow solid. MS: m/z=223.0 [M+H]⁺.

### Intermediate 26: 2-carbamoyl-5-fluorobenzo[d]thiazole-7-carboxylic acid

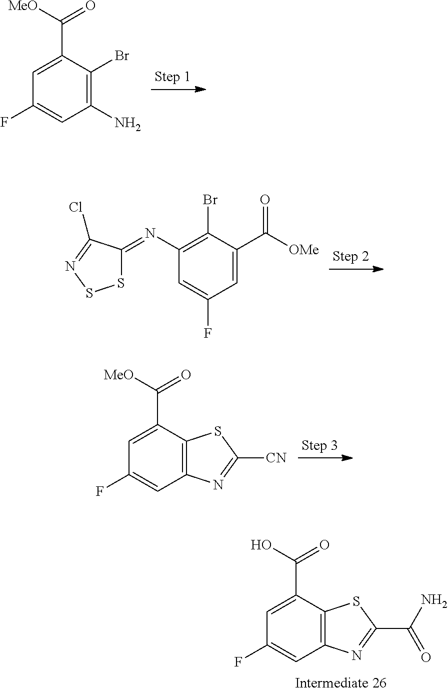

### Step 1: Methyl (Z)-2-bromo-3-((4-chloro-5H-1,2,3-dithiazol-5-ylidene)amino)-5-fluorobenzoate

A mixture of 4,5-dichlorodithiazol-2-ium;chloride (2.19 g, 10.48 mmol) and methyl 3-amino-2-bromo-5-fluoro-benzoate (2.0 g, 8.06 mmol) in DCM (30 mL) was stirred at 25° C. for 3 hr, and then Py (1.28 g, 16.13 mmol) was added dropwise to the solution, the reaction mixture was stirred for another 2 hr. The reaction mixture was concentrated under reduced pressure to give a residue. Methyl (Z)-2-bromo-3-((4-chloro-5H-1,2,3-dithiazol-5-ylidene)amino)-5-fluorobenzoate (3.0 g, yield: 92%) was obtained as a yellow soild, which was directly used to the next step without further purification. MS: m/z=384.5 [M+H]⁺.

### Step 2: Methyl 2-cyano-5-fluorobenzo[d]thiazole-7-carboxylate

To a solution of methyl 2-bromo-3-[(Z)-(4-chlorodithiazol-5-ylidene)amino]-5-fluoro-benzoate (1.0 g, 2.61 mmol) in pyridine (15 mL) was added CuI (496 mg, 2.61 mmol). The mixture was stirred under microwave irradiation (400 W) 115° C. for 0.5 hr. The reaction was diluted with H₂O (40 mL) and extracted with EtOAc (30 mL×3). The combined organic layers were washed with 0.5 M HCl (30 mL×3) and brine (10 mL×5), dried over Na₂SO₄, filtered, and concentrated under reduced pressure to give a residue. The residue was purified by silica gel flash chromatography (Eluent of 2˜3% EtOAc in petroleum ether), methyl 2-cyano-5-fluorobenzo[d]thiazole-7-carboxylate (117 mg, yield: 19%) was obtained as white solid, which was directly used to the next step. MS: m/z=236.9 [M+H]+. ¹H NMR (400 MHz, Chloroform-d) δ 8.13-8.07 (m, 1H), 4.07 (s, 3H. F NMR (400 MHz, Chloroform-d) δ −111.7.

### Step 3: 2-Carbamoyl-5-fluorobenzo[d]thiazole-7-carboxylic acid

A mixture of methyl 2-cyano-5-fluorobenzo[d]thiazole-7-carboxylate (400 mg, 1.69 mmol), LiOH·H₂O (81.1 mg, 3.39 mmol) in THF (2 mL) and H₂O (2 mL) was stirred at 60° C. for 0.5 hr. The reaction was concentrated under reduced pressure to give a residue, 2-carbamoyl-5-fluorobenzo[d]thiazole-7-carboxylic acid (Intermediate 26, 350 mg, yield: 88%) was obtained as light-yellow solid, which was directly used to the next step without further purification. MS: m/z=240.8 [M+H]⁺. ¹H NMR (400 MHz, Dimethylsulfoxide-d₆) δ 8.30 (s, 1H), 7.91 (s, 1H), 7.83-7.72 (m, 1H), 7.65-7.62 (m, 1H). ¹F NMR (400 MHz, Dimethylsulfoxide-d₆) δ −116.1.

### Intermediate 27: 2-Carbamoyl-6-fluorobenzo[d]thiazole-4-carboxylic acid

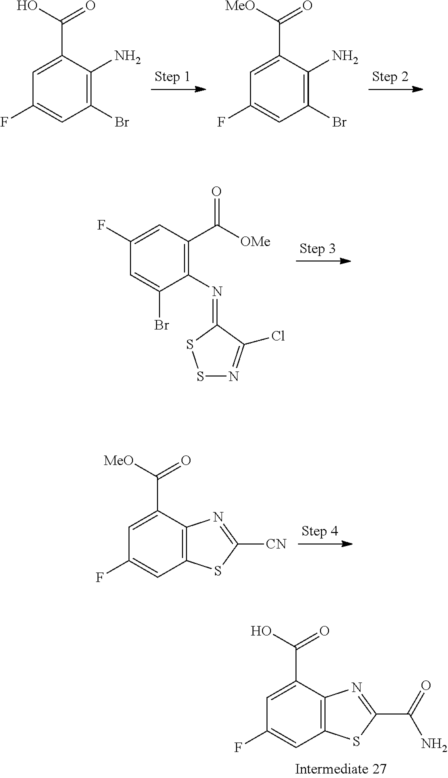 [SMILES: Nc1c(Br)cc(F)cc1C(=O)O ; COC(=O)c1cc(F)cc(Br)c1N ; COC(=O)c1cc(F)cc(Br)c1/N=c1\ssnc1Cl ; COC(=O)c1cc(F)cc2sc(C#N)nc12 ; NC(=O)c1nc2c(C(=O)O)cc(F)cc2s1]

### Step 1: Methyl 2-amino-3-bromo-5-fluorobenzoate

To a solution of 2-amino-3-bromo-5-fluoro-benzoic acid (2.0 g, 8.55 mmol) in MeOH (50 mL) was added SOCl₂ (3.05 g, 25.64 mmol) dropwise. The mixture was stirred at 70° C. for 2 hr. The reaction mixture was concentrated under reduced pressure to give methyl 2-amino-3-bromo-5-fluorobenzoate (2.0 g, yield: 73%) was obtained as a black solid, which was directly used to the next step without further purification. MS: m/z=249.8, 250.8 [M+H]+. ¹H NMR (400 MHz, Chloroform-d) δ 7.68-7.53 (m, 1H), 7.42-7.40 (m, 1H), 5.34 (br s, 2H), 3.90 (s, 3H). ¹F NMR (400 MHz, Chloroform-d) 6-127.5.

### Step 2: Methyl (Z)-3-bromo-2-((4-chloro-5H-1,2,3-dithiazol-5-ylidene)amino)-5-fluorobenzoate

A mixture of 4,5-dichlorodithiazol-2-ium;chloride (168.1 mg, 806 μmol) and methyl 2-amino-3-bromo-5-fluorobenzoate (200 mg, 806 μmol) in DCM (3 mL) was stirred at 25° C. for 3 hr, and then pyridine (128 mg, 1.61 mmol) was added dropwise to the solution, the reaction mixture was stirred for another 2 hr. The reaction mixture was concentrated under reduced pressure to give a residue. Methyl (Z)-3-bromo-2-((4-chloro-5H-1,2,3-dithiazol-5-ylidene)amino)-5-fluorobenzoate (300 mg, yield. 63%) was obtained as light-green oil, which was used directly used to the next step without further purification. MS: m/z=384.4 [M+H]⁺.

### Step 3: Methyl 2-cyano-6-fluorobenzo[d]thiazole-4-carboxylate

To a solution of methyl methyl (Z)-3-bromo-2-((4-chloro-5H-1,2,3-dithiazol-5-ylidene)amino)-5-fluorobenzoate (200 mg, 521 μmol) in pyridine (4 mL) was added Cul (99 mg, 521 μmol). The mixture was stirred under microwave irradiation (400 W) 115° C. for 0.5 hr. The reaction was diluted with H₂O (20 mL) and extracted with EtOAc (10 mL×3). The combined organic layers were washed with 0.5 M HCl (5 mL×3) and brine (10 mL×5), dried over Na₂SO₄, filtered, and concentrated under reduced pressure to give a residue. The residue was purified by silica gel flash chromatography (Eluent of 6˜8% Ethyl acetate in petroleum ether), Methyl 2-cyano-6-fluorobenzo[d]thiazole-4-carboxylate (65 mg, yield: 51%) was obtained as white solid, which was used directly used to the next step. MS: m/z=236.7 [M+H]⁺. ¹H NMR (400 MHz, Dimethylsulfoxide-d₆) δ 8.01 (dd, J=9.2, 2.8 Hz, 1H), 7.87 (dd, J=7.2, 2.8 Hz, 1H), 4.08 (s, 3H). ¹F NMR (400 MHz, Chloroform-d) δ −108.7.

### Step 4: 2-Carbamoyl-6-fluorobenzo[d]thiazole-4-carboxylic acid

A mixture of methyl 2-cyano-6-fluorobenzo[d]thiazole-4-carboxylate (20 mg, 85 μmol), LiOH·H₂O (3 mg, 85 μmol) in THF (1 mL) and H₂O (1 mL) was stirred at 60° C. for 0.5 hr. The reaction was concentrated under reduced pressure to give 2-carbamoyl-6-fluorobenzo[d]thiazole-4-carboxylic acid (Intermediate 27, 15 mg, yield: 68%) was obtained as white solid, which was used directly used to the next step without further purification. MS: m/z=240.6 [M+H]⁺. 1H NMR (400 MHz, Dimethylsulfoxide-d₆) δ 8.37 (br s, 1H), 8.07 (br s, 1H), 7.90 (dd, J=8.4, 3.2 Hz, 1H), 7.41-7.34 (m, 1H). ¹F NMR (400 MHz, Chloroform-d) 6-114.2.

### Intermediate 28: 3-(3-(4-(((3S,5S)-3,5-Dimethylpiperazin-1-yl)methyl)phenyl)-5-phenyl-3H-imidazo[4,5-b]pyridin-2-yl)pyridin-2-amine

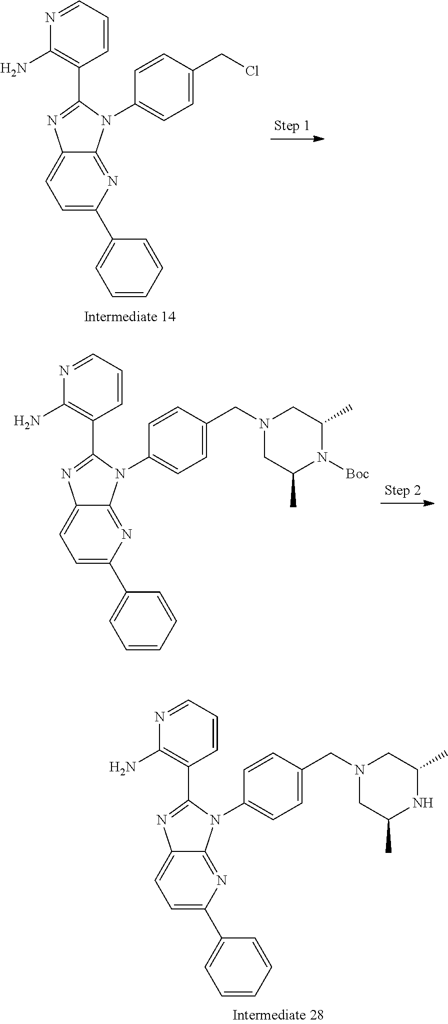 [SMILES: Nc1ncccc1-c1nc2ccc(-c3ccccc3)nc2n1-c1ccc(CCl)cc1 ; C[C@H]1CN(Cc2ccc(-n3c(-c4cccnc4N)nc4ccc(-c5ccccc5)nc43)cc2)C[C@H](C)N1C(=O)OC(C)(C)C ; C[C@H]1CN(Cc2ccc(-n3c(-c4cccnc4N)nc4ccc(-c5ccccc5)nc43)cc2)C[C@H](C)N1]

### Step 1: Tert-butyl (2S,6S)-4-(4-(2-(2-aminopyridin-3-yl)-5-phenyl-3H-imidazo[4,5-b]pyridin-3-yl)benzyl)-2,6-dimethylpiperazine-1-carboxylate

To a solution of Intermediate 14 (1.0 g, 2.43 mmol) in DMF (4 mL) were added K₂CO₃ (671 mg, 4.86 mmol) and tert-butyl (2R,6R)-2,6-dimethylpiperazine-1-carboxylate (624 mg, 2.91 mmol). The mixture was stirred at 25° C. for 16 hr. The reaction mixture was quenched with H₂O (10 mL) at 25° C., and then diluted with CH₂Cl₂ (6 mL) and extracted with H₂O (20 mL×3). The combined organic layers were washed with 20 mL brine, dried over Na₂SO₄, filtered, and concentrated under reduced pressure to give a residue. The residue was purified by flash chromatography on silica gel (Eluent of 0˜10% MeOH in CH₂Cl₂), tert-butyl (2S,6S)-4-(4-(2-(2-aminopyridin-3-yl)-5-phenyl-3H-imidazo[4,5-b]pyridin-3-yl)benzyl)-2,6-dimethylpiperazine-1-carboxylate (750 mg, yield: 52%) was obtained as a yellow solid. MS: m/z=590.3 [M+H]⁺. ¹H NMR (400 MHz, Dimethylsulfoxide-d₆) 68.26 (d, J=8.4 Hz, 1H), 8.04-7.95 (m, 4H), 7.51-7.38 (m, 7H), 7.16 (dd, J=7.6, 1.6 Hz, 1H), 7.04 (br s, 2H), 6.38 (dd, J=8.0, 4.8 Hz, 1H), 3.82-3.75 (m, 2H), 3.64 (d, J=13.6 Hz, 1H), 3.47 (d, J=13.6 Hz, 1H), 2.90-2.86 (m, 1H), 2.76-2.70 (m, 1H), 2.26-2.18 (m, 2H), 1.40 (s, 9H), 1.22 (d, J=6.4 Hz, 6H).

### Step 2: 3-(3-(4-(((3S,5S)-3,5-Dimethylpiperazin-1-yl)methyl)phenyl)-5-phenyl-3H-imidazo[4,5-b]pyridin-2-yl)pyridin-2-amine

A solution of tert-butyl (2S,6S)-4-(4-(2-(2-aminopyridin-3-yl)-5-phenyl-3H-imidazo[4,5-b]pyridin-3-yl)benzyl)-2,6-dimethylpiperazine-1-carboxylate (700 mg, 1.19 mmol) in HCl/1,4-dioxane (10 mL) was stirred at 25° C. for 2 hr. The reaction was filtered and concentrated under reduced pressure to give 3-(3-(4-(((3S,5S)-3,5-dimethylpiperazin-1-yl)methyl)phenyl)-5-phenyl-3H-imidazo[4,5-b]pyridin-2-yl)pyridin-2-amine (Intermediate 28, 540 mg, HCl salt, yield: 86%) as a white solid MS: m/z=490.6 [M+H]⁺. ¹H NMR (400 MHz, Dimethylsulfoxide-d₆) δ 8.27 (d, J=8.4 Hz, 1H), 8.08-7.95 (m, 4H), 7.52-7.35 (m, 7H), 7.15 (dd, J=7.6, 1.6 Hz, 1H), 7.04 (br s, 2H), 6.40-6.34 (m, 1H), 3.54 (d, J=14.0 Hz, 1H), 3.45-3.40 (m, 1H), 3.10-3.04 (m, 2H), 2.41-2.33 (m, 2H), 2.06-2.00 (m, 2H), 1.04 (d, J=6.4 Hz, 6H).

### Intermediate 29: 3-(3-(4-(((3R,5R)-3,5-dimethylpiperazin-1-yl)methyl)phenyl)-5-phenyl-3H-imidazo[4,5-b]pyridin-2-yl)pyridin-2-amine

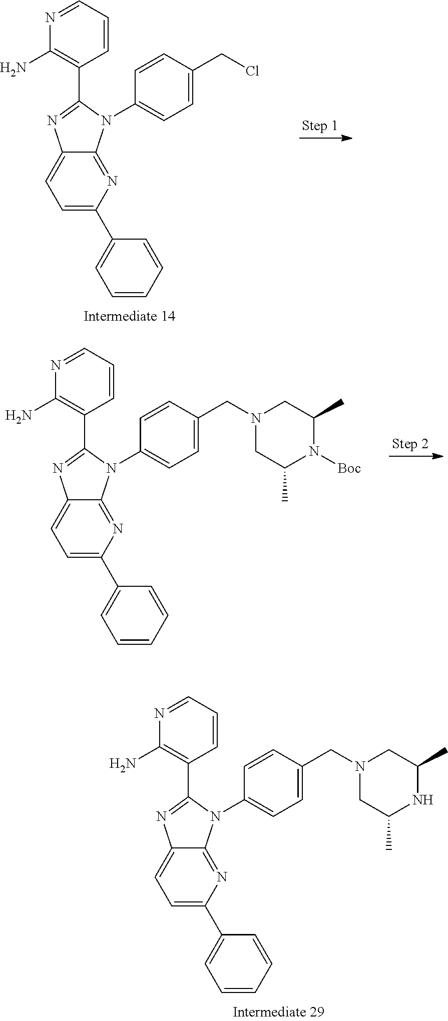 [SMILES: Nc1ncccc1-c1nc2ccc(-c3ccccc3)nc2n1-c1ccc(CCl)cc1 ; C[C@@H]1CN(Cc2ccc(-n3c(-c4cccnc4N)nc4ccc(-c5ccccc5)nc43)cc2)C[C@@H](C)N1C(=O)OC(C)(C)C ; C[C@@H]1CN(Cc2ccc(-n3c(-c4cccnc4N)nc4ccc(-c5ccccc5)nc43)cc2)C[C@@H](C)N1]

### Step 1: Tert-butyl (2R,6R)-4-(4-(2-(2-aminopyridin-3-yl)-5-phenyl-3H-imidazo[4,5-b]pyridin-3-yl)benzyl)-2,6-dimethylpiperazine-1-carboxylate

To a solution of Intermediate 14 (1.06 g, 2.57 mmol), tert-butyl (2R,6R)-2,6-dimethylpiperazine-1-carboxylate (500 mg, 2.33 mmol) in DMF (10 mL) was added DIEA (905 mg, 7.0 mmol). The mixture was stirred at 80° C. for 16 hr. The mixture was quenched with H₂O (40 mL) and extracted with EtOAc (40 mL×3), the combined organic layers were dried over anhydrous Na₂SO₄, filtered, and concentrated to give a residue. The residue was purified by silica gel flash chromatography (Eluent of 0˜9% MeOH in CH₂Cl₂), tert-butyl (2R,6R)-4-(4-(2-(2-aminopyridin-3-yl)-5-phenyl-3H-imidazo[4,5-b]pyridin-3-yl)benzyl)-2,6-dimethylpiperazine-1-carboxylate (Intermediate 29, 1 g, yield: 69.4%) was obtained as a yellow solid. MS: m/z=590.4 [M+H]⁺. ¹H NMR (400 MHz, Dimethylsulfoxide-d₆) 68.27 (d, J=8.4 Hz, 1H), 8.05-7.98 (m, 4H), 7.52-7.43 (m, 6H), 7.42-7.36 (m, 1H), 7.16 (dd, J=7.6, 1.6 Hz, 1H), 7.04 (br s, 2H), 6.37 (dd, J=7.6, 4.8 Hz, 1H), 3.83-3.74 (m, 2H), 3.65 (d, J=13.6 Hz, 1H), 3.48 (d, J=13.6 Hz, 1H), 3.35-3.39 (m, 2H), 2.27-2.19 (m, 2H), 1.42-1.41 (m, 1H), 1.40 (s, 9H), 1.23 (d, J=6.4 Hz, 6H).

### Step 2: 3-(3-(4-(((3R,5R)-3,5-Dimethylpiperazin-1-yl)methyl)phenyl)-5-phenyl-3H-imidazo[4,5-b]pyridin-2-yl)pyridin-2-amine

A solution of tert-butyl (2R,6R)-4-(4-(2-(2-aminopyridin-3-yl)-5-phenyl-3H-imidazo[4,5-b]pyridin-3-yl)benzyl)-2,6-dimethylpiperazine-1-carboxylate (100 mg, 169 μmol) in HCl/1,4-dioxane (4 M, 1 mL) was stirred at 25° C. for 0.5 hr. The mixture was filtered to give 3-(3-(4-(((3R,5R)-3,5-dimethylpiperazin-1-yl)methyl)phenyl)-5-phenyl-3H-imidazo[4,5-b]pyridin-2-yl)pyridin-2-amine (Intermediate 29, 84 mg HCl salt, yield: 95%) as a yellow solid. MS: m/z=490.2 [M+H]+. ¹H NMR (400 MHz, Dimethylsulfoxide-d₆) δ 12.41-11.57 (m, 0.5H), 10.79-10.27 (m, 0.5H), 10.07-9.71 (m, 1H), 8.65-8.51 (m, 1H), 8.38 (d, J=8.4 Hz, 1H), 8.16 (d, J=6.0 Hz, 1H), 8.11-7.95 (m, 4H), 7.93-7.82 (m, 3H), 7.68 (d, J=8.0 Hz, 2H), 7.51-7.42 (m, 3H), 7.04 (t, J=6.8 Hz, 1H), 4.50-4.25 (m, 2H), 3.44-3.32 (m, 2H), 3.20-3.01 (m, 2H), 2.91-2.87 (m, 1H), 2.75-2.71 (m, 1H), 1.60-1.31 (m, 6H).

### Intermediate 30: 3-[3-[4-(4,7-Diazaspiro[2.5]octan-7-ylmethyl)phenyl]-5-phenyl-imidazo[4,5-b]pyridin-2-yl]pyridin-2-amine

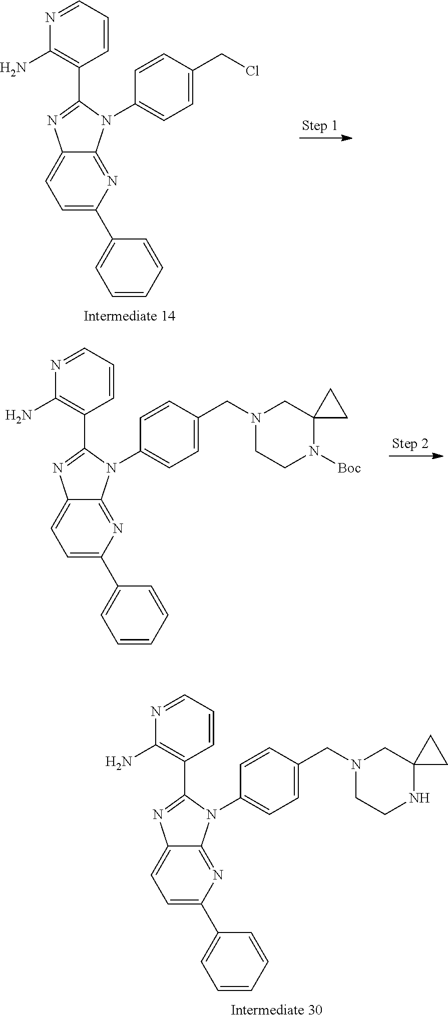 [SMILES: Nc1ncccc1-c1nc2ccc(-c3ccccc3)nc2n1-c1ccc(CCl)cc1 ; CC(C)(C)OC(=O)N1CCN(Cc2ccc(-n3c(-c4cccnc4N)nc4ccc(-c5ccccc5)nc43)cc2)CC12CC2 ; Nc1ncccc1-c1nc2ccc(-c3ccccc3)nc2n1-c1ccc(CN2CCNC3(CC3)C2)cc1]

### Step 1: Tert-butyl 7-(4-(2-(2-aminopyridin-3-yl)-5-phenyl-3H-imidazo[4,5-b]pyridin-3-yl)benzyl)-4,7-diazaspiro[2.5]octane-4-carboxylate

To a solution of Intermediate 14 (1.0 g, 2.43 mmol) and tert-butyl 4,7-diazaspiro[2.5]octane-4-carboxylate (618 mg, 2.91 mmol) in DMF (10 mL) was added K₂CO₃ (671 mg, 4.86 mmol). The mixture was stirred at 25° C. for 24 hr. The reaction mixture was diluted with H₂O (10 mL) at 25° C. and extracted with CH₂Cl₂ (20 mL×3). The combined organic layers were washed with brine (15 mL), dried over Na₂SO₄, filtered, and concentrated under reduced pressure to give a residue. The residue was purified by silica gel flash chromatography (Eluent of 0˜60% EtOAc in petroleum ether) to give tert-butyl 7-[[4-[2-(2-amino-3-pyridyl)-5-phenyl-imidazo[4,5-b]pyridin-3-yl]phenyl]methyl]-4,7-diazaspiro[2.5]octane-4-carboxylate (610 mg, yield: 38%) as a yellow solid, which was used directly in the next step without further purification. MS: m/z=588.1 [M+H]⁺.

### Step 2: 3-(3-(4-(4,7-Diazaspiro[2.5]octan-7-ylmethyl)phenyl)-5-phenyl-3H-imidazo[4,5-b]pyridin-2-yl)pyridin-2-amine

To a solution of tert-butyl 7-[[4-[2-(2-amino-3-pyridyl)-5-phenyl-imidazo[4,5-b]pyridin-3-yl]phenyl]methyl]-4,7-diazaspiro[2.5]octane-4-carboxylate (500 mg, 0.851 mmol) in 1,4-dioxane (3 mL) was added HCl/1,4-dioxane (4 M, 5 mL). The mixture was stirred at 25° C. for 1 hr. The reaction mixture was concentrated under reduced pressure to remove 1,4-dioxane. The crude was dissolved in H₂O (5 mL) and extracted with CH₂Cl₂ (5 mL×3), the aqueous phase was added NaHCO₃ and extracted with CH₂Cl₂ (5 mL×3), filtered and concentrated to give 3-[3-[4-(4,7-diazaspiro[2.5]octan-7-ylmethyl)phenyl]-5-phenyl-imidazo[4,5-b]pyridin-2-yl]pyridin-2-amine (Intermediate 30, 350 mg, yield: 81%) as a yellow solid. MS: m/z=488.3 [M+H]⁺. ¹H NMR (400 MHz, Dimethylsulfoxide-d₆) δ 8.26 (d, J=8.4 Hz, 1H), 8.09-7.92 (m, 4H), 7.56-7.33 (m, 7H), 7.14 (dd, J=7.8, 1.8 Hz, 1H), 7.05 (br s, 2H), 6.34 (dd, J=7.6, 4.8 Hz, 1H), 3.52 (s, 2H), 2.76 (t, J=4.8 Hz, 2H), 2.44-2.32 (m, 2H), 2.18 (s, 2H), 0.47-0.38 (m, 2H), 0.35-0.25 (m, 2H).

### Intermediate 31: 3-(3-(4-((3,8-Diazabicyclo[3.2.1]octan-3-yl)methyl)phenyl)-5-phenyl-3H-imidazo[4,5-b]pyridin-2-yl)pyridin-2-amine

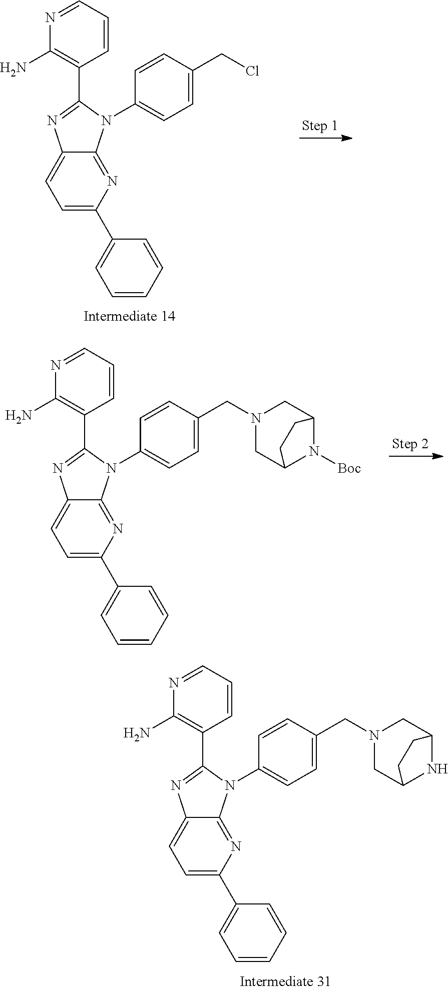

### Step 1: Tert-butyl 3-(4-(2-(2-aminopyridin-3-yl)-5-phenyl-3H-imidazo[4,5-b]pyridin-3-yl)benzyl)-3,8-diazabicyclo[3.2.1]octane-8-carboxylate

To a solution of Intermediate 14 (500 mg, 1.21 mmol) and tert-butyl 3,8-diazabicyclo[3.2.1]octane-8-carboxylate (309 mg, 1.46 mmol) in DMF (8 mL) was added K₂CO₃ (336 mg, 2.43 mmol). The mixture was stirred at 25° C. for 8 hr. The reaction mixture was diluted with H₂O (10 mL) at 25° C. and extracted with CH₂Cl₂ (20 mL×3). The combined organic layers were washed with brine (30 mL), dried over Na₂SO₄, filtered, and concentrated under reduced pressure to give a residue. The residue was purified by flash chromatography on silica gel (Eluent of 0˜10% EtOAc in petroleum ether), tert-butyl 3-(4-(2-(2-aminopyridin-3-yl)-5-phenyl-3H-imidazo[4,5-b]pyridin-3-yl)benzyl)-3,8-diazabicyclo[3.2.1]octane-8-carboxylate (450 mg, yield: 63%) as a yellow solid. MS: m/z=588.3 [M+H]⁺.

### Step 2: 3-(3-(4-((3,8-Diazabicyclo[3.2.1]octan-3-yl)methyl)phenyl)-5-phenyl-3H-imidazo[4,5-b]pyridin-2-yl)pyridin-2-amine

To a solution of tert-butyl 3-(4-(2-(2-aminopyridin-3-yl)-5-phenyl-3H-imidazo[4,5-b]pyridin-3-yl)benzyl)-3,8-diazabicyclo[3.2.1]octane-8-carboxylate (450 mg, 0.766 mmol) in 1,4-dioxane (5 mL) was added HCl/1,4-dioxane (4 M, 5 mL) at 25° C. The mixture was stirred at 25° C. for 6 hr. The reaction was filtered and concentrated under reduced pressure to give 3-(3-(4-((3,8-diazabicyclo[3.2.1]octan-3-yl)methyl)phenyl)-5-phenyl-3H-imidazo[4,5-b]pyridin-2-yl)pyridin-2-amine (Intermediate 31, 240 mg, HCl salt) as a light yellow solid. MS: m/z=488.1 [M+H]⁺. ¹H NMR (400 MHz, Methanol-d₄) δ 8.34-8.28 (m, 1H), 8.05-7.95 (m, 4H), 7.90-7.85 (m, 3H), 7.65 (d, J=8.0 Hz, 2H), 7.48-7.37 (m, 3H), 6.90 (t, J=6.8 Hz, 1H), 4.35-4.25 (m, 4H), 3.50-3.40 (m, 4H), 2.41 (d, J=8.4 Hz, 2H), 2.25-2.16 (m, 2H).

### Intermediate 32: 3-(3-(4-((3,8-diazabicyclo[3.2.1]octan-8-yl)methyl)phenyl)-5-phenyl-3H-imidazo[4,5-b]pyridin-2-yl)pyridin-2-amine

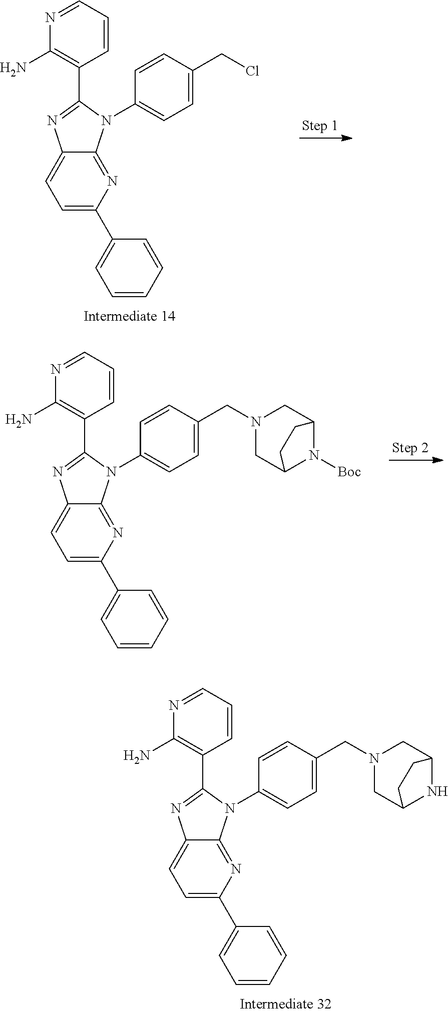 [SMILES: Nc1ncccc1-c1nc2ccc(-c3ccccc3)nc2n1-c1ccc(CCl)cc1 ; CC(C)(C)OC(=O)N1C2CCC1CN(Cc1ccc(-n3c(-c4cccnc4N)nc4ccc(-c5ccccc5)nc43)cc1)C2 ; Nc1ncccc1-c1nc2ccc(-c3ccccc3)nc2n1-c1ccc(CN2CC3CCC(C2)N3)cc1]

### Step 1: Tert-butyl 8-(4-(2-(2-aminopyridin-3-yl)-5-phenyl-3H-imidazo[4,5-b]pyridin-3-yl)benzyl)-3,8-diazabicyclo[3.2.1]octane-3-carboxylate

To a solution of Intermediate 14 (1.0 g, 2.43 mmol) and tert-butyl 3,8-diazabicyclo[3.2.1]octane-3-carboxylate (618 mg, 2.91 mmol) in DMF (15 mL) was added K₂CO₃ (671 mg, 4.86 mmol). The mixture was stirred at 25° C. for 8 hr. The reaction mixture was diluted with H₂O (10 mL) at 25° C. and extracted with CH₂Cl₂ (20 mL×3). The combined organic layers were washed with brine (30 mL), dried over Na₂SO₄, filtered and concentrated under reduced pressure to give a residue. The residue was purified by flash chromatography on silica gel (Eluent of 0˜10% EtOAc in petroleum ether), tert-butyl 8-(4-(2-(2-aminopyridin-3-yl)-5-phenyl-3H-imidazo[4,5-b]pyridin-3-yl)benzyl)-3,8-diazabicyclo[3.2.1]octane-3-carboxylate (956 mg, yield: 67%) was obtained as a yellow solid. MS: m/z=588.2 [M+H]⁺. ¹H NMR (400 MHz, Dimethylsulfoxide-d₆) 88.26 (d, J=8.4 Hz, 1H), 8.05-7.95 (m, 4H), 7.55 (d, J=8.4 Hz, 2H), 7.48-7.37 (m, 5H), 7.16 (dd, J=7.6, 1.6 Hz, 1H), 7.01 (br s, 2H), 6.38 (dd, J=7.6, 4.8 Hz, 1H), 3.59 (s, 2H), 3.20-2.85 (m, 6H), 2.02-1.92 (m, 2H), 1.54-1.48 (m, 2H), 1.40 (s, 9H).

### Step 2: 3-(3-(4-((3,8-Diazabicyclo[3.2.1]octan-8-yl)methyl)phenyl)-5-phenyl-3H-imidazo[4,5-b]pyridin-2-yl)pyridin-2-amine

To a solution of tert-butyl 8-(4-(2-(2-aminopyridin-3-yl)-5-phenyl-3H-imidazo[4,5-b]pyridin-3-yl)benzyl)-3,8-diazabicyclo[3.2.1]octane-3-carboxylate (600 mg, 1.02 mmol) in 1,4-dioxane (5 mL) was added HCl/1,4-dioxane (4 M, 5 mL) at 25° C. The mixture was stirred at 25° C. for 6 hr. The reaction was filtered and concentrated under reduced pressure to give 3-(3-(4-((3,8-diazabicyclo[3.2.1]octan-8-yl)methyl)phenyl)-5-phenyl-3H-imidazo[4,5-b]pyridin-2-yl)pyridin-2-amine (Intermediate 32, 535 mg, HCl salt) as a light yellow solid. MS: m/z=488.2 [M+H]⁺. ¹H NMR (400 MHz, Dimethylsulfoxide-d₆) δ 12.42 (br s, 1H), 10.4-10.0 (m, 2H), 8.55 (br s, 1H), 8.38 (d, J=8.0 Hz, 1H), 8.13-8.07 (m, 1H), 8.06-8.00 (m, 5H), 7.98-7.94 (m, 1H), 7.69 (d, J=8.0 Hz, 2H), 7.50-7.40 (m, 3H), 6.94 (t, J=7.2 Hz, 1H), 4.39-4.33 (m, 2H), 4.01 (s, 2H), 3.95-3.89 (m, 3H), 3.42-3.39 (m, 3H), 2.47-2.38 (m, 2H).

### Intermediate 33: 3-(4-(2-(2-Aminopyridin-3-yl)-5-phenyl-3H-imidazo[4,5-b]pyridin-3-yl)benzyl)-3-azabicyclo[3.2.1]octan-8-amine

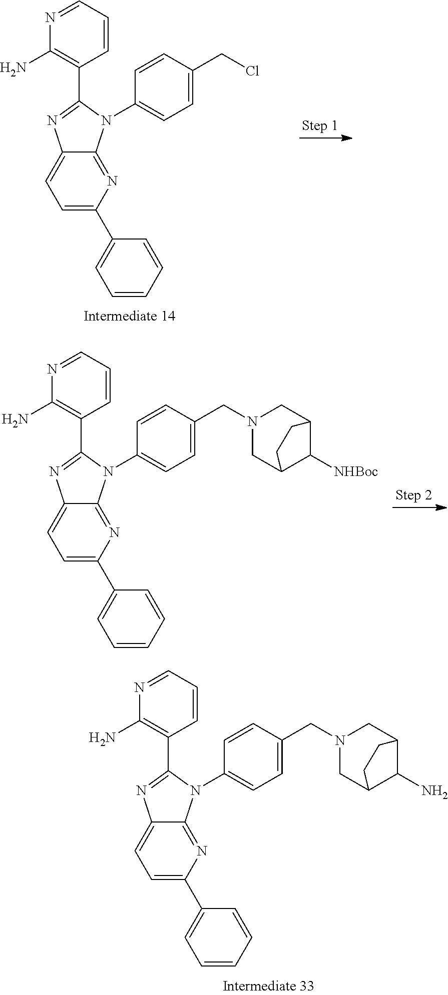

### Step 1: Tert-butyl (3-(4-(2-(2-aminopyridin-3-yl)-5-phenyl-3H-imidazo[4,5-b]pyridin-3-yl)benzyl)-3-azabicyclo[3.2.1]octan-8-yl)carbamate

To a solution of Intermediate 14 and tert-butyl (3-azabicyclo[3.2.1]octan-8-yl)carbamate (549 mg, 2.43 mmol) in DMF (10 mL) were added K₂CO₃ (1.01 g, 7.28 mmol) and NaI (36.4 mg, 243 μmol). The resulting mixture was stirred at 25° C. for 14 hr. Then the reaction mixture was diluted with H₂O (50 mL) and extracted with CH₂Cl₂ (50 mL×2). The combined organic layers were dried over Na₂SO₄, filtered and concentrated under reduced pressure to give a residue. The crude product was purified by column chromatography (Eluent of 30˜50% ethyl acetate in petroleum ether), tert-butyl (3-(4-(2-(2-aminopyridin-3-yl)-5-phenyl-3H-imidazo[4,5-b]pyridin-3-yl)benzyl)-3-azabicyclo[3.2.1]octan-8-yl)carbamate (500 mg, yield: 32%) was obtained as a yellow solid. MS: m/z=602.3 [M+H]⁺. ¹H NMR (400 MHz, Dimethylsulfoxide-d₆) δ 8.26 (d, J=8.4 Hz, 1H), 8.04-7.94 (m, 4H), 7.50-7.39 (m, 7H), 7.15 (d, J=7.2 Hz, 1H), 7.05 (br s, 2H), 6.92-6.79 (m, 1H), 6.41-6.32 (m, 1H), 3.60-3.51 (m, 2H), 2.66-2.63 (m, 1H), 2.38-2.33 (dd, J=10.4, 2.8 Hz, 1H), 2.15 (d, J=10.4 Hz, 1H), 2.10-2.02 (m, 2H), 1.74-1.57 (m, 4H), 1.42-1.37 (m, 9H), 1.30-1.18 (m, 2H).

### Step 2: 3-(4-(2-(2-Aminopyridin-3-yl)-5-phenyl-3H-imidazo[4,5-b]pyridin-3-yl)benzyl)-3-azabicyclo[3.2.1]octan-8-amine

To a solution of tert-butyl (3-(4-(2-(2-aminopyridin-3-yl)-5-phenyl-3H-imidazo[4,5-b]pyridin-3-yl)benzyl)-3-azabicyclo[3.2.1]octan-8-yl)carbamate (500 mg, 831 μmol) in 1,4-dioxane (2 mL) was added HCl/dioxane (2 mL). The mixture was stirred at 25° C. for 0.5 hr. The reaction mixture was diluted with H₂O (10 mL) and extracted with 10 mL EtOAc. The aqueous layer was alkalized with NaHCO₃ to pH around 10, then extracted with EtOAc (20 mL×2). The combined organic layers were dried over Na₂SO₄, filtered and concentrated under reduced pressure to give a residue. The crude product was purified by column chromatography (Eluent of 5˜10% MeOH in CH₂Cl₂), 3-(4-(2-(2-aminopyridin-3-yl)-5-phenyl-3H-imidazo[4,5-b]pyridin-3-yl)benzyl)-3-azabicyclo[3.2.1]octan-8-amine (Intermediate 33, 270 mg, yield: 62%) was obtained as a yellow solid. MS: m/z=502.3 [M+H]⁺. ¹H NMR (400 MHz, Methanol-d₄)) δ 8.17 (d, J=8.4 Hz, 1H), 8.02 (d, 1=7.2 Hz, 2H), 7.97 (dd, J=5.2, 1.6 Hz, 1H), 7.92 (d, J=8.4 Hz, 1H), 7.51 (d, J=8.4 Hz, 2H), 7.43-7.35 (m, 5H), 7.29 (dd, J=7.6, 1.6 Hz, 1H), 6.44 (dd, J=7.6, 4.8 Hz, 1H), 3.62 (s, 2H), 2.96 (t, J=4.4 Hz, 1H), 2.57-2.49 (m, 4H), 1.93-1.88 (m, 2H), 1.85-1.79 (m, 2H), 1.75-1.68 (m, 2H)

### Intermediate 34: (S)-3-(3-(4-((3-methylpiperazin-1-yl)methyl)phenyl)-5-phenyl-3H-imidazo[4,5-b]pyridin-2-yl)pyridin-2-amine

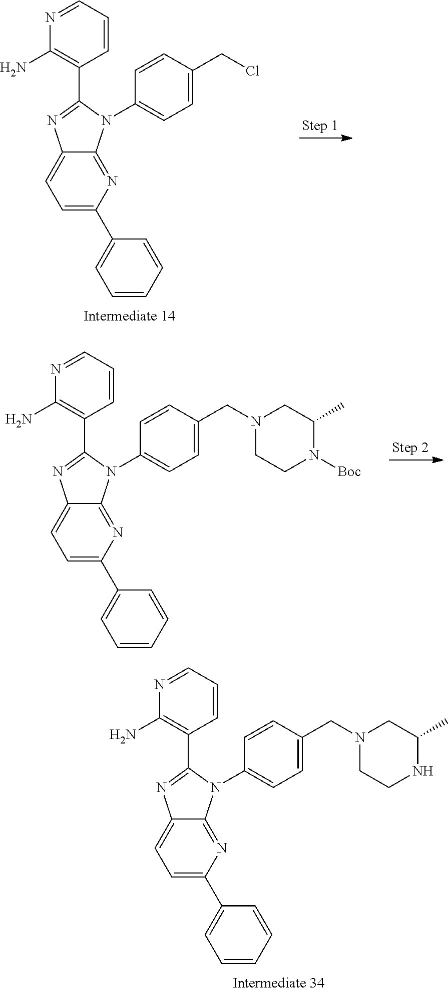

### Step 1: (S)-Tert-butyl 4-(4-(2-(2-aminopyridin-3-yl)-5-phenyl-3H-imidazo[4,5-b]pyridin-3-yl)benzyl)-2-methylpiperazine-1-carboxylate

To a solution of Intermediate 14 (1.0 g, 2.43 mmol) and (S)-tert-butyl 2-methylpiperazine-1-carboxylate (583 mg, 2.91 mmol) in DMF (10 mL) was added K₂CO₃ (671 mg, 4.86 mmol). The mixture was stirred at 25° C. for 24 hr. The reaction mixture was diluted with H₂O (10 mL) at 25° C. and extracted with CH₂Cl₂ (20 mL×3). The combined organic layers were washed with (15 mL), dried over Na₂SO₄, filtered, and concentrated under reduced pressure to give a residue. The residue was purified by silica gel flash chromatography (Eluent of 0˜60% EtOAc in petroleum ether) to give tert-butyl tert-butyl (S)-4-(4-(2-(2-aminopyridin-3-yl)-5-phenyl-3H-imidazo[4,5-b]pyridin-3-yl)benzyl)-2-methylpiperazine-1-carboxylate (550 mg, yield: 35%) as a yellow solid, which was used directly in the next step without further purification. MS: m/z=576.1 [M+H]r.

### Step 2: (S)-3-(3-(4-((3-Methylpiperazin-1-yl)methyl)phenyl)-5-phenyl-3H-imidazo[4,5-b]pyridin-2-yl)pyridin-2-amine

To a solution of (S)-4-(4-(2-(2-aminopyridin-3-yl)-5-phenyl-3H-imidazo[4,5-b]pyridin-3-yl)benzyl)-2-methylpiperazine-1-carboxylate (500 mg, 0.869 mmol) in HCl/1,4-dioxane (4 M, 5 mL) was added. The mixture was stirred at 25° C. for 1 hr. The reaction mixture was concentrated under reduced pressure to remove 1,4-dioxane. The crude product was triturated with MeOH (2 mL) at 25° C. for 0.5 hr to give (S)-3-(3-(4-((3-methylpiperazin-1-yl)methyl)phenyl)-5-phenyl-3H-imidazo[4,5-b]pyridin-2-yl)pyridin-2-amine (Intermediate 34, 350 mg, yield: 82%) as a yellow solid. MS: m/z=476.2 [M+H]⁺. ¹H NMR (400 MHz, Methanol-d₄) δ 8.32 (d, J=8.4 Hz, 1H), 8.07-7.99 (m, 4H), 7.94-7.84 (m, 3H), 7.73 (d, J=8.4 Hz, 2H), 7.50-7.37 (m, 3H), 6.96-6.88 (m, 1H), 4.57 (s, 1H), 4.60-4.53 (s, 2H), 3.94-3.83 (m, 1H), 3.81-3.69 (m, 3H), 3.68-3.56 (m, 1H), 3.54-3.41 (m, 1H), 3.39-3.31 (m, 1H), 1.45 (d, J=6.8 Hz, 3H).

### Intermediate 35: 3-(3-(4-((1,4-Diazepan-1-yl)methyl)phenyl)-5-phenyl-3H-imidazo[4,5-b]pyridin-2-yl)pyridin-2-amine

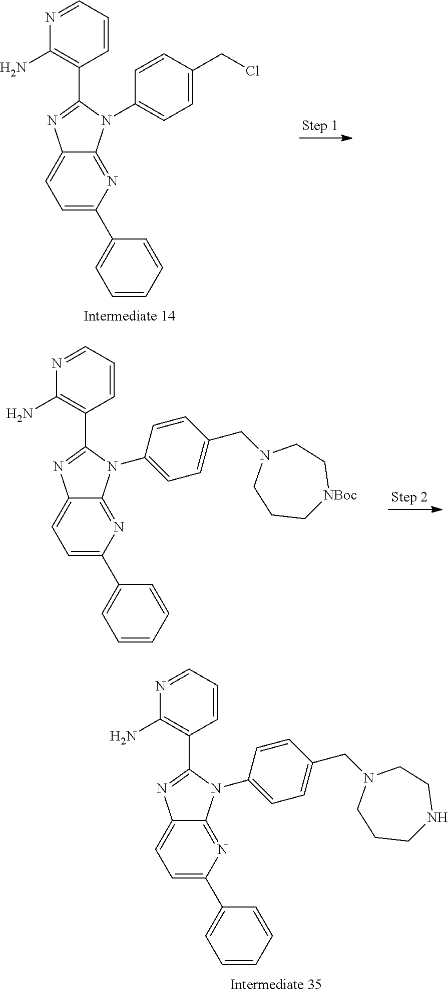

### Step 1: Tert-butyl 4-(4-(2-(2-aminopyridin-3-yl)-5-phenyl-3H-imidazo[4,5-b]pyridin-3-yl)benzyl)-1,4-diazepane-1-carboxylate

To a solution of Intermediate 14 (1 g, 2.43 mmol), tert-butyl 1,4-diazepane-1-carboxylate (584 mg, 2.92 mmol) in ACN (20 mL) was added NaI (36.4 mg, 243 μmol) and K₂CO₃ (671 mg, 4.86 mmol). The mixture was stirred at 80° C. for 2 hr. The reaction mixture was concentrated directly. The residue was purified by silica gel flash chromatography (Eluent of 0˜10% MeOH in CH₂Cl₂) to give tert-butyl 4-[[4-[2-(2-amino-3-pyridyl)-5-phenyl-imidazo[4,5-b]pyridin-3-yl]phenyl]methyl]-1,4-diazepane-1-carboxylate (520 mg, yield: 33%) as a light-yellow solid, which was used directly in the next step without further purification. MS: m/z=576.4 [M+H]⁺.

### Step 2: 3-(3-(4-((1,4-Diazepan-1-yl)methyl)phenyl)-5-phenyl-3H-imidazo[4,5-b]pyridin-2-yl)pyridin-2-amine

To a solution of tert-butyl 4-[[4-[2-(2-amino-3-pyridyl)-5-phenyl-imidazo[4,5-b]pyridin-3-yl]phenyl]methyl]-1,4-diazepane-1-carboxylate (520 mg, 0.903 mmol) in HCl/1,4-dioxane (4 M, 10 mL). The mixture was stirred at 25° C. for 1 hr. The reaction mixture was concentrated directly to give the crude product (310 mg HCl salt, yield: 67%). The 100 mg was purified by prep-HPLC (column: Phenomenex C18 150×25 mm×10 um; mobile phase: [water (NH₄HCO₃)-ACN]; B %: 28%-58%, 8 min) to give 3-[3-[4-(1,4-diazepan-1-ylmethyl)phenyl]-5-phenyl-imidazo[4,5-b]pyridin-2-yl]pyridin-2-amine (Intermediate 35, 9 mg) as light-yellow solid. MS: m/z=476.3 [M+H]⁺. ¹H NMR (400 MHz, Dimethylsulfoxide-d₆) δ 8.4 (d, J=8.4 Hz, 1H), 8.04-7.97 (m, 4H), 7.52-7.39 (m, 7H), 7.14 (dd, J=7.6, 1.8 Hz, 1H), 7.05 (br s, 2H), 6.39-6.34 (m, 1H), 3.72 (s, 2H), 2.82 (t, J=6.4 Hz, 2H), 2.78-2.74 (m, 2H), 2.67 (t, 0.1=6.4 Hz, 2H), 2.66-2.57 (m, 2H), 1.78-1.64 (m, 2H).

### Intermediate 36: 3-(3-(4-((4-(Methylamino)piperidin-1-yl)methyl)phenyl)-5-phenyl-3H-imidazo[4,5-b]pyridin-2-yl)pyridin-2-amine

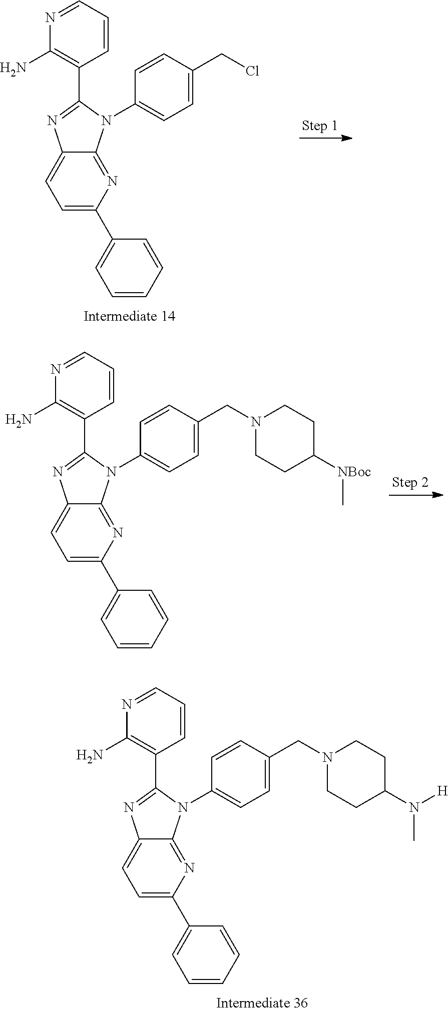

### Step 1: 3-[3-[4-[[4-(methylamino)-1-piperidyl]methyl]phenyl]-5-phenyl-imidazo[4,5-b]pyridin-2-yl]pyridin-2-amine

To a solution of Intermediate 14 (300 mg, 0.73 mmol) and tert-butyl N-methyl-N-(4-piperidyl)carbamate (312 mg, 1.46 mmol) in DMF (2 mL) were added NaI (18.2 mg, 0.073 mmol) and K₂CO₃ (336 mg, 2.43 mmol). The mixture was stirred at 80° C. for 2 hr. After cooling to 20° C., the reaction mixture was poured into H₂O (3 mL), extracted with EtOAc (10 mL×3). The combined organic layers were dried over anhydrous Na₂SO₄, filtered, and concentrated under reduced pressure to give a residue. The residue was purified by silica gel flash chromatography (Eluent of 0˜7% MeOH in CH₂Cl₂) to give tert-butyl N-[1-[[4-[2-(2-amino-3-pyridyl)-5-phenyl-imidazo[4,5-b]pyridin-3-yl]phenyl]methyl]-4-piperidyl]-N-methyl-carbamate (220 mg, yield: 51%,) as a yellow solid. MS: m/z=590.3 [M+H]⁺.

### Step 2: 3-(3-(4-((4-(Methylamino)piperidin-1-yl)methyl)phenyl)-5-phenyl-3H-imidazo[4,5-b]pyridin-2-yl)pyridin-2-amine

To a solution of tert-butyl N-[1-[[4-[2-(2-amino-3-pyridyl)-5-phenyl-imidazo[4,5-b]pyridin-3-yl]phenyl]methyl]-4-piperidyl]-N-methyl-carbamate (200 mg, 0.339 mmol) in CH₂Cl₂ (2 mL) was added TFA (38.7 mg, 0.339 mmol). The mixture was stirred at 25° C. for 2 hr. The reaction mixture was concentrated directly to give a residue (185 mg TFA salt, yield: 93%). The 50 mg crude product was diluted with aqueous NaHCO₃ (10 mL) and extracted with CH₂Cl₂ (10 mL×3). and then purified by prep-TLC (CH₂Cl₂:MeOH=10:1) to give 3-[3-[4-[[4-(methylamino)-1-piperidyl]methyl]phenyl]-5-phenyl-imidazo[4,5-b]pyridin-2-yl]pyridin-2-amine (Intermediate 36) as an off-white solid. MS: m/z=490.2 [M+H]⁺. ¹H NMR (400 MHz, Dimethylsulfoxide-d₆) δ 8.27 (d, J=8.4 Hz, 1H), 8.07-7.94 (m, 4H), 7.53-7.43 (m, 6H), 7.41-7.39 (m, 1H), 7.16 (dd, J=7.8, 1.2 Hz, 1H), 7.02 (br s, 2H), 6.37 (dd, J=7.4, 4.8 Hz, 1H), 3.55 (s, 2H), 3.47-3.37 (m, 1H), 2.80 (br d, J=12.0 Hz, 2H), 2.31 (s, 3H), 2.01 (dd, J=11.4, 9.8 Hz, 2H), 1.85-1.77 (m, 2H), 1.36-1.22 (m, 2H).

### Intermediate 37: 3-(3-(4-(((1R,4R)-2,5-Diazabicyclo[2.2.1]heptan-2-yl)methyl)phenyl)-5-phenyl-3H-imidazo[4,5-b]pyridin-2-yl)pyridin-2-amine

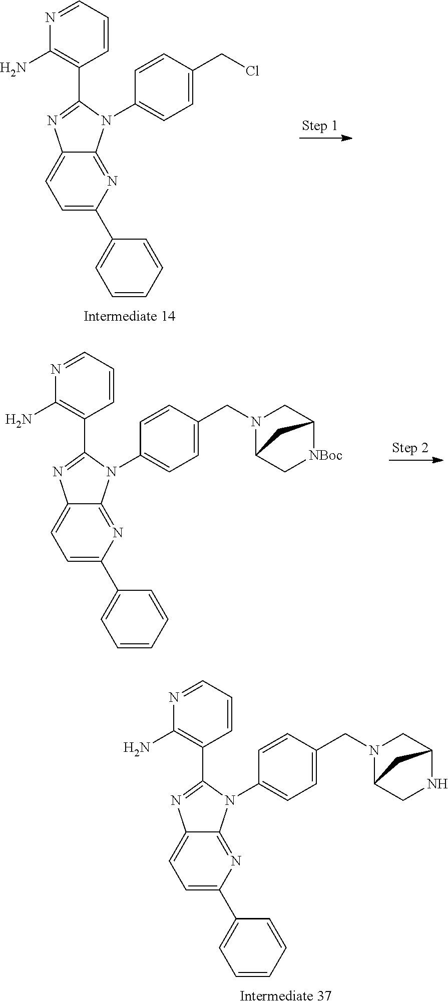 [SMILES: Nc1ncccc1-c1nc2ccc(-c3ccccc3)nc2n1-c1ccc(CCl)cc1 ; CC(C)(C)OC(=O)N1C[C@H]2C[C@@H]1CN2Cc1ccc(-n2c(-c3cccnc3N)nc3ccc(-c4ccccc4)nc32)cc1 ; Nc1ncccc1-c1nc2ccc(-c3ccccc3)nc2n1-c1ccc(CN2C[C@H]3C[C@@H]2CN3)cc1]

### Step 1: Tert-butyl (1R,4R)-5-(4-(2-(2-aminopyridin-3-yl)-5-phenyl-3H-imidazo[4,5-b]pyridin-3-yl)benzyl)-2,5-diazabicyclo[2.2.1]heptane-2-carboxylate

To a solution of Intermediate 14 (1.0 g, 2.43 mmol) and tert-butyl (1R,4R)-2,5-diazabicyclo[2.2.1]heptane-2-carboxylate (578 mg, 2.91 mmol) in DMF (15 mL) was added K₂CO₃ (671 mg, 4.86 mmol). The mixture was stirred at 25° C. for 16 hr. The reaction mixture was quenched with H₂O (10 mL) at 25° C. and extracted with EtOAc (50 mL×3). The combined organic layers were washed with brine (20 mL×3), dried over Na₂SO₄, filtered, and concentrated under reduced pressure to give a residue. The residue was purified by silica gel flash chromatography ((Eluent of 0˜10% MeOH in CH₂Cl₂), tert-butyl (1R,4R)-5-(4-(2-(2-aminopyridin-3-yl)-5-phenyl-3H-imidazo[4,5-b]pyridin-3-yl)benzyl)-2,5-diazabicyclo[2.2.1]heptane-2-carboxylate (1.0 g, yield: 72%) was obtained as a yellow solid. MS: m/z=574.2 [M+H]⁺.

### Step 2: 3-(3-(4-(((1R,4R)-2,5-Diazabicyclo[2.2.1]heptan-2-yl)methyl)phenyl)-5-phenyl-3H-imidazo[4,5-b]pyridin-2-yl)pyridin-2-amine

To a solution of tert-butyl (1R,4R)-5-(4-(2-(2-aminopyridin-3-yl)-5-phenyl-3H-imidazo[4,5-b]pyridin-3-yl)benzyl)-2,5-diazabicyclo[2.2.1]heptane-2-carboxylate (1.0 g, 1.74 mmol) in 1,4-dioxane (2 mL) was added HCl/1,4-dioxane (4 M, 10 mL). The mixture was stirred at 25° C. for 2 hr. The reaction mixture was concentrated under reduced pressure to remove 1,4-dioxane. 3-(3-(4-(((1R,4R)-2,5-Diazabicyclo[2.2.1]heptan-2-yl)methyl)phenyl)-5-phenyl-3H-imidazo[4,5-b]pyridin-2-yl)pyridin-2-amine (Intermediate 37, 854 mg, 2HCl salt, yield: 90%) was obtained as a yellow solid. MS: m/z=474.2 [M+H]⁺. ¹H NMR (400 MHz, Methanol-d₄) δ 8.33 (d, J=8.4 Hz, 1H), 8.06-8.00 (m, 6H), 7.88 (dd, J=7.6, 1.6 Hz, 1H), 7.73 (d, J=8.4 Hz, 2H), 7.46-7.40 (m, 3H), 6.92 (dd, J=7.6, 6.4 Hz, 1H), 4.84-4.81 (m, 1H), 4.75-4.65 (m, 2H), 4.14 (d, J=12.8 Hz, 1H), 3.96-3.92 (m, 1H), 3.74-3.71 (m, 1H), 3.62-3.60 (m, 1H), 3.38-3.32 (m, 1H), 2.87-2.82 (m, 1H), 2.39-2.36 (m, 1H).

### Intermediate 38: 3-(3-(4-((2,5-Diazabicyclo[2.2.2]octan-2-yl)methyl)phenyl)-5-phenyl-3H-imidazo[4,5-b]pyridin-2-yl)pyridin-2-amine

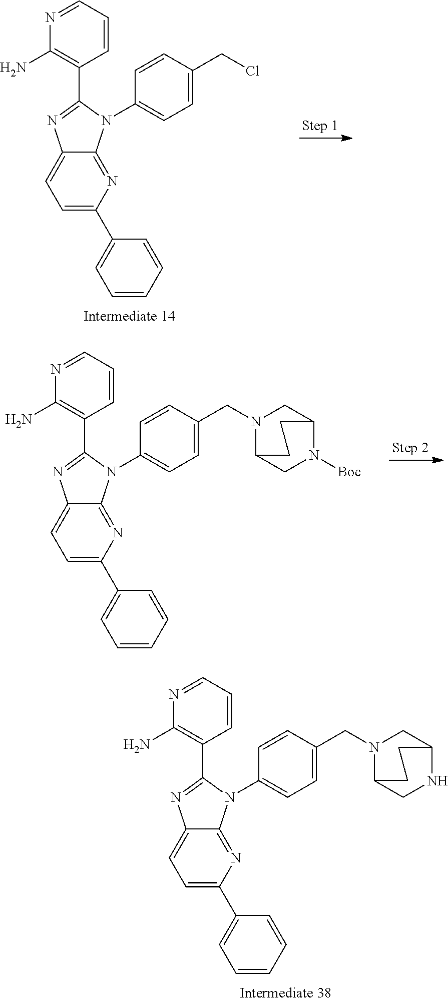 [SMILES: Nc1ncccc1-c1nc2ccc(-c3ccccc3)nc2n1-c1ccc(CCl)cc1 ; CC(C)(C)OC(=O)N1CC2CCC1CN2Cc1ccc(-n2c(-c3cccnc3N)nc3ccc(-c4ccccc4)nc32)cc1 ; Nc1ncccc1-c1nc2ccc(-c3ccccc3)nc2n1-c1ccc(CN2CC3CCC2CN3)cc1]

### Step 1: Tert-butyl 5-(4-(2-(2-aminopyridin-3-yl)-5-phenyl-3H-imidazo[4,5-b]pyridin-3-yl)benzyl)-2,5-diazabicyclo[2.2.2]octane-2-carboxylate

To a solution of Intermediate 14 (1.0 g, 2.43 mmol) and tert-butyl 2,5-diazabicyclo[2.2.2]octane-2-carboxylate (567 mg, 2.67 mmol) in MeCN (10 mL) was added K₂CO₃ (1.0 mg, 7.28 mmol,) and NaI (109 mg, 728 μmol). The mixture was stirred at 25° C. for 16 hr. The reaction mixture was diluted with H₂O (10 mL) at 25° C. and extracted with CH₂Cl₂ (10 mL×3). The combined organic layers were washed with brine (30 mL), dried over Na₂SO₄, filtered, and concentrated under reduced pressure to give a residue. The crude product was purified by flash chromatography on silica gel (Eluent of 0˜5% MeOH in CH₂Cl₂), tert-butyl 5-(4-(2-(2-aminopyridin-3-yl)-5-phenyl-3H-imidazo[4,5-b]pyridin-3-yl)benzyl)-2,5-diazabicyclo[2.2.2]octane-2-carboxylate (700 mg, yield: 45%) was obtained as a yellow solid. MS: m/z=588.3 [M+H]⁺. ¹H NMR (400 MHz, Dimethylsulfoxide-d₆) 68.26 (d, J=8.4 Hz, 1H), 8.06-7.95 (m, 4H), 7.54-7.36 (m, 7H), 7.15 (dd, J=8.0, 1.6 Hz, 1H), 7.02 (s, 2H), 6.38 (dd, J=7.6, 4.8 Hz, 1H), 3.94-3.72 (m, 3H), 3.69-3.54 (m, 1H), 3.27-3.20 (m, 1H), 3.17 (d, J=5.2 Hz, 0.0.5H), 2.90-2.86 (m, 0.5H), 2.84-2.79 (m, 2H), 2.07-1.95 (m, 1H), 1.78-1.69 (m, 2H), 1.63-1.52 (m, 1H), 1.45-1.37 (d, J=9.6 Hz, 9H).

### Step 2: 3-(3-(4-((2,5-Diazabicyclo[2.2.2]octan-2-yl)methyl)phenyl)-5-phenyl-3H-imidazo[4,5-b]pyridin-2-yl)pyridin-2-amine

To a solution of tert-butyl 5-(4-(2-(2-aminopyridin-3-yl)-5-phenyl-3H-imidazo[4,5-b]pyridin-3-yl)benzyl)-2,5-diazabicyclo[2.2.2]octane-2-carboxylate (50 mg, 85 μmol) in 4 M HCl in 1,4-dioxane (1 mL). The mixture was stirred at 25° C. for 2 hr. The mixture was then filtered, the collected solid residue was washed with 1,4-dioxane (10 mL×2) and dried in vacuo to give 3-(3-(4-((2,5-diazabicyclo[2.2.2]octan-2-yl)methyl)phenyl)-5-phenyl-3H-imidazo[4,5-b]pyridin-2-yl)pyridin-2-amine (Intermediate 38, 44.9 mg, yield: 99%) as a yellow solid. MS: m/z=488.2 [M+H]⁺. ¹H NMR (400 MHz, Dimethylsulfoxide-d₄) δ 12.11-11.44 (m, 1H), 10.27-9.61 (m, 2H), 8.56-8.40 (m, 1H), 8.38 (d, J=8.4 Hz, 1H), 8.15 (dd, J=6.0, 1.2 Hz, 1H), 8.10-7.99 (m, 5H), 7.86 (dd, J=7.2, 1.2 Hz, 1H), 7.68 (d, J=8.4 Hz, 2H), 7.51-7.46 (m, 2H), 7.46-7.40 (m, 1H), 6.91 (dd, J=7.6, 6.4 Hz, 1H), 4.62 (br s, 2H), 4.01-3.79 (m, 4H), 3.76-3.70 (m, 2H), 2.21 (m, 2H), 1.97-1.80 (m, 2H).

### Intermediate 39: 2-(4-Piperidylamino)pyridine-4-carbonitrile

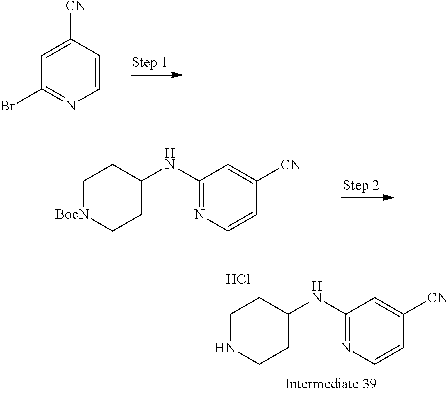 [SMILES: N#Cc1ccnc(Br)c1 ; CC(C)(C)OC(=O)N1CCC(Nc2cc(C#N)ccn2)CC1 ; Cl ; N#Cc1ccnc(NC2CCNCC2)c1]

### Step 1: Tert-butyl 4-[(4-cyano-2-pyridyl)amino]piperidine-1-carboxylate

A mixture of tert-butyl 4-aminopiperidine-1-carboxylate (1.20 g, 6.01 mmol), 2-bromopyridine-4-carbonitrile (1.0 g, 5.46 mmol), BINAP (136 mg, 219 μmol), Pd₂(dba)₃ (100 mg, 109 μmol) and t-BuONa (1.05 g, 10.9 mmol) in toluene (20 mL) was degassed and purged with N₂ three times, and then the mixture was stirred at 100° C. for 2 hr under N₂ atmosphere. The mixture was diluted with 50 mL H₂O and extracted with EtOAc (50 mL 2). The combined organic layers were washed with 50 mL brine, dried over Na₂SO₄, filtered and concentrated under reduced pressure to give a residue. The crude product was purified by flash chromatography on silica gel (Eluent of 0˜25% EtOAc in petroleum ether), tert-butyl 4-[(4-cyano-2-pyridyl)amino]piperidine-1-carboxylate (350 mg, yield: 20%) was obtained as a yellow solid. ¹H NMR (400 MHz, Chloroform-d) δ 8.18 (d, J=5.2 Hz, 1H), 6.71 (dd, J=5.2, 1.2 Hz, 1H), 6.56 (s, 1H), 4.72 (d, J=7.6 Hz, 1H), 4.14-4.00 (m, 2H), 3.90-3.76 (m, 1H), 2.96-2.90 (m, 2H), 2.05-1.99 (m, 2H), 1.46 (s, 9H), 1.43-1.33 (m, 2H).

### Step 2: 2-(4-Piperidylamino)pyridine-4-carbonitrile

A solution of tert-butyl 4-[(4-cyano-2-pyridyl)amino]piperidine-1-carboxylate (350 mg, 1.16 mmol) in HCl in 1,4-dioxane (4 M, 5 mL). The mixture was stirred at 25° C. for 1 hr. The reaction mixture was concentrated under reduced pressure to give 2-(4-piperidylamino)pyridine-4-carbonitrile (Intermediate 39, 200 mg, HCl salt, yield: 72%) as a yellow solid, which was used to the next step without further purification.

### Intermediate 40: 3-(3-(4-((4-Aminopiperidin-1-yl)methyl-d₂)phenyl)-5-phenyl-3H-imidazo[4,5-b]pyridin-2-yl)pyridin-2-amine hydrochloride

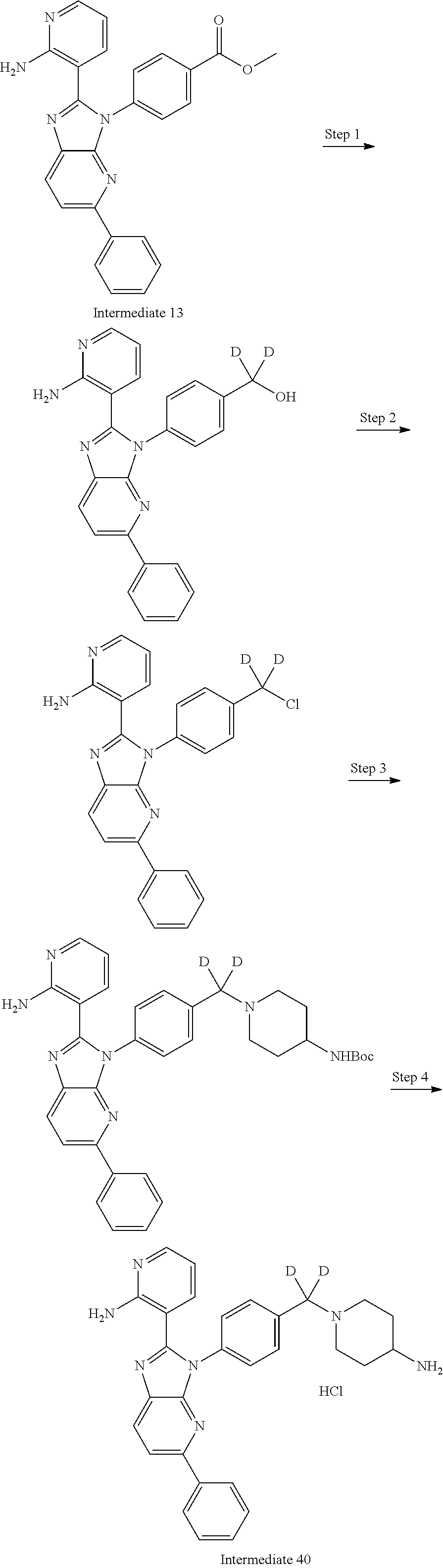

### Step 1: (4-(2-(2-Aminopyridin-3-yl)-5-phenyl-3H-imidazo[4,5-b]pyridin-3-yl)phenyl)methan-d2-ol

To a solution of Intermediate 13 (500 mg, 1.19 mmol) in THF (50 mL) at 0° C. was added LiAlD₄ (99.6 mg, 2.37 mmol) in portions under N₂ atmosphere. The mixture was stirred at 0° C. for 1 hr. The reaction mixture was quenched with Na₂SO₄·10 H₂O at 0° C., filtered and concentrated under reduced pressure. The residue was purified by silica gel flash chromatography (Eluent of 0˜100% EtOAc in petroleum ether) to give (4-(2-(2-aminopyridin-3-yl)-5-phenyl-3H-imidazo[4,5-b]pyridin-3-yl)phenyl)methan-d₂-ol (190 mg, yield: 41%) as a yellow solid. MS: m/z=396.1 [M+H]⁺. ¹H NMR (400 MHz, Chloroform-d) δ 8.15 (d, J=8.4 Hz, 1H), 8.03-7.97 (m, 3H), 7.83 (d, J=8.4 Hz, 1H), 7.57 (d, J=8.4 Hz, 2H), 7.47-7.37 (m, 6H), 6.44 (dd, J=7.6, 5.2 Hz, 1H).

### Step 2: 3-(3-(4-(Chloromethyl-d₂)phenyl)-5-phenyl-3H-imidazo[4,5-b]pyridin-2-yl)pyridin-2-amine

To a solution of (4-(2-(2-aminopyridin-3-yl)-5-phenyl-3H-imidazo[4,5-b]pyridin-3-yl)phenyl)methan-d₂-ol (190 mg, 480 μmol) in CH₂Cl₂ (5 mL) was added SOCl₂ (171 mg, 1.44 mmol) in portions under N₂ atmosphere. The mixture was stirred at 40° C. for 1 hr. The reaction mixture was filtered. The filter liquor was concentrated to dryness. The crude was used in the next step without further purification. 3-(3-(4-(chloromethyl-d₂)phenyl)-5-phenyl-3H-imidazo[4,5-b]pyridin-2-yl)pyridin-2-amine (198 mg, crude) was obtained as a yellow solid. MS: m/z=414.0 [M+H]⁺. ¹H NMR (400 MHz, Dimethylsulfoxide-d₆) δ 8.36 (d, J=8.4 Hz, 1H), 8.12 (dd, J=6.4, 1.6 Hz, 1H), 8.09-8.04 (m, 3H), 7.90-7.87 (m, 1H), 7.68-7.56 (m, 4H), 7.51-7.41 (m, 3H), 6.90 (dd, 1=7.2, 6.4 Hz, 1H).

### Step 3: tert-Butyl(1-((4-(2-(2-aminopyridin-3-yl)-5-phenyl-3H-imidazo[4,5-b]pyridin-3-yl)phenyl)methyl-d₂)piperidin-4-yl)carbamate

A solution of 3-(3-(4-(chloromethyl-d₂)phenyl)-5-phenyl-3H-imidazo[4,5-b]pyridin-2-yl)pyridin-2-amine (248 mg, 599 μmol), tert-butyl N-(4-piperidyl)carbamate (132 mg, 659 μmol), K₂CO₃ (166 mg, 1.20 mmol) and NaI (89.8 mg, 599.2 μmol) in DMF (3 mL) was stirred at 80° C. for 2 h under N₂ atmosphere. The water (10 mL) was added to the mixture, and the mixture was extracted with EtOAc (3×5 mL). The combined organic layers were washed with brine (10 mL), dried over Na₂SO₄, filtered, and concentrated. The residue was purified by silica gel flash chromatography (Eluent of 0˜1000% EtOAc in petroleum ether) and prep-HPLC (column: Xtimate C18 100*30 mm*3 μm, mobile phase: [water (FA)-ACN]; B %: 20%-60%, 9 min). tert-Butyl(1-((4-(2-(2-aminopyridin-3-yl)-5-phenyl-3H-imidazo[4,5-b]pyridin-3-yl)phenyl)methyl-d₂)piperidin-4-yl)carbamate (180 mg, yield: 52%) was obtained as a yellow solid. MS: m/z=578.2 [M+H]⁺. ¹H NMR (400 MHz, Chloroform-d) δ 8.13 (d, J=8.4 Hz, 1H), 8.07 (dd, J=4.8, 1.6 Hz, 1H), 8.03-8.00 (m, 2H), 7.81 (d, J=8.4 Hz, 1H), 7.63-7.52 (m, 2H), 7.46-7.42 (m, 4H), 7.40-7.36 (m, 1H), 7.08 (dd, J=7.6, 1.6 Hz, 1H), 6.59 (br s, 2H), 6.36 (dd, J=7.6, 4.8 Hz, 1H), 4.52-4.39 (br s, 1H), 3.57-3.59 (m, 1H), 3.01-2.98 (m, 1H), 2.33-2.28 (m, 2H), 2.00-1.98 (m, 2H), 1.68-1.65 (m, 2H), 1.47-1.46 (m, 1H), 1.44 (s, 9H).

### Step 4: 3-(3-(4-((4-Aminopiperidin-1-yl)methyl-d₂)phenyl)-5-phenyl-3H-imidazo[4,5-b]pyridin-2-yl)pyridin-2-amine hydrochloride

To a solution of tert-butyl (1-((4-(2-(2-aminopyridin-3-yl)-5-phenyl-3H-imidazo[4,5-b]pyridin-3-yl)phenyl)methyl-d₂)piperidin-4-yl)carbamate (90.0 mg, 156 μmol) in CH₂Cl₂ (3 mL) was added HCl in 1,4-dioxane (4 M, 974 μL) under N₂ atmosphere. The mixture was stirred at 20° C. for 1 hr. The mixture was filtered, washed with CH₂Cl₂ (2×10 mL). The filter liquor was concentrated to dryness. Filter cake was dried to remove solvent. The crude product was used in the next step without further purification. 3-(3-(4-((4-Aminopiperidin-1-yl)methyl-d2)phenyl)-5-phenyl-3H-imidazo[4,5-b]pyridin-2-yl)pyridin-2-amine hydrochloride (Intermediate 40, 70 mg, HCl salt, yield: 93%) was obtained as a yellow solid. MS: m/z=478.2 [M+H]⁺. ¹H NMR (400 MHz, Methanol-d₄) δ 8.33 (d, J=8.4 Hz, 1H), 8.08-8.00 (m, 4H), 7.91 (d, J=8.0 Hz, 3H), 7.73 (d, J=9.2 Hz, 2H), 7.50-7.38 (m, 3H), 6.91 (t, J=6.6 Hz, 1H), 3.70 (d, J=12.0 Hz, 2H), 3.61-3.52 (m, 1H), 3.35-3.29 (m, 1H), 3.28-3.17 (m, 1H), 2.31 (d, J=10.8 Hz, 2H), 2.23-2.04 (m, 2H).

### Intermediate 41: 3-(3-(4-((4-((Methyl-d₃)amino)piperidin-1-yl)methyl)phenyl)-5-phenyl-3H-imidazo[4,5-b]pyridin-2-yl)pyridin-2-amine

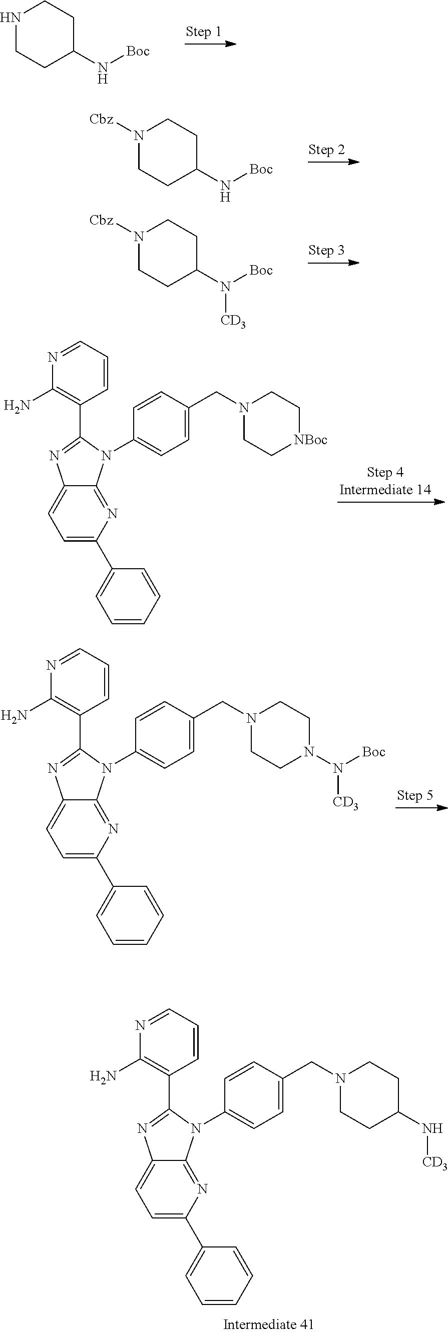

### Step 1: Benzyl 4-((tert-butoxycarbonyl)amino)piperidine-1-carboxylate

To a solution of tert-butyl N-(4-piperidyl)carbamate (12 g, 59.9 mmol) in CH₂Cl₂ (100 mL) was added TEA (18.2 g, 179 mmol), and then the CbzCl (11.2 g, 65.9 mmol) was added into the mixture at 0° C. The mixture was stirred at 25° C. for 2 hr. The mixture was concentrated under reduced pressure. The crude was purified by silica gel flash chromatography (Eluent of 10˜50% EtOAc in petroleum ether) to give benzyl 4-((tert-butoxycarbonyl)amino)piperidine-1-carboxylate (16 g, yield: 71%) as an off-white solid. ¹H NMR (400 MHz, Dimethylsulfoxide-d₆) δ 7.40-7.28 (m, 5H), 5.06 (s, 2H), 3.90 (d, J=13.6 Hz, 2H), 3.50-3.34 (m, 2H), 2.89 (s, 2H), 1.71 (d, J=10.8 Hz, 2H), 1.37 (s, 9H), 1.30-1.18 (m, 2H).

### Step 2: Benzyl 4-((tert-butoxycarbonyl)(methyl-d₃)amino)piperidine-1-carboxylate

To a solution of benzyl 4-(tert-butoxycarbonylamino)piperidine-1-carboxylate (13 g, 38.9 mmol) in THF (200 mL) was added NaH (4.66 g, 117 mmol) at 0° C. After stirring at 0° C. for 30 min, CD₃I (16.5 g, 117 mmol) was added to the mixture. The mixture was stirred at 25° C. for 16 hr. The mixture was quenched with NH₄Cl (aq) (100 mL) at 0° C. The mixture was diluted with H₂O (100 mL) and extrated with CH₂Cl₂ (200 mL×2). The combined organic layers were washed with brine (200 mL×2), dried over Na₂SO₄, filtered and concentrated under reduced pressure. The residue was purified by silica gel flash chromatography (Eluent of 0˜20% EtOAc in petroleum ether) to give benzyl 4-((tert-butoxycarbonyl)(methyl-d₃)amino)piperidine-1-carboxylate (8.6 g, yield: 56%) as a colorless oil. MS: m/z=252.3 [M+H−100]⁺. ¹H NMR (400 MHz, Dimethylsulfoxide-d₆) 57.42-7.28 (m, 5H), 5.07 (s, 2H), 4.14-3.89 (m, 3H), 2.82 (s, 2H), 1.58-1.48 (m, 4H), 1.39 (s, 9H).

### Step 3: tert-Butyl (methyl-d₃)(piperidin-4-yl)carbamate

To a solution of benzyl 4-[tert-butoxycarbonyl(trideuteriomethyl)amino]piperidine-1-carboxylate (8.6 g, 24.5 mmol) in MeOH (90 mL) was added Pd/C (900 mg, 24.5 mmol). The mixture was stirred at 25° C. for 16 hr under H₂ (15 psi). The mixture was filtered, and the filter cake was washed with MeOH (30 mL). The filtrate was concentrated under reduced pressure to give a tert-butyl (methyl-d₃)(piperidin-4-yl)carbamate (5 g, yield: 80%) as a colorless oil. MS: m/z=218.3 [M+H]⁺. ¹H NMR (400 MHz, Dimethylsulfoxide-d₆) δ 3.93-3.62 (m, 1H), 3.26-3.16 (m, 1H), 2.95 (d, J=12.0 Hz, 2H), 2.47-2.38 (m, 2H), 1.54-1.42 (m, 4H), 1.39 (s, 9H).

### Step 4: tert-Butyl (1-(4-(2-(2-aminopyridin-3-yl)-5-phenyl-3H-imidazo[4,5-b]pyridin-3-yl)benzyl)piperidin-4-yl)(methyl-d₃)carbamate

To a solution of Intermediate 14 (1 g, 2.43 mmol) in DMF (10 mL) were added tert-butyl (methyl-d₃)(piperidin-4-yl)carbamate (527 mg, 2.43 mmol), NaI (182 mg, 1.21 mmol) and K₂CO₃ (1.0 g, 7.28 mmol). The mixture was stirred at 80° C. for 18 hr. The reaction mixture was diluted with H₂O (20 mL) and extracted with CH₂Cl₂ (20 mL×3). The combined organic layers were washed with brine (20 mL×2), dried over Na₂SO₄, filtered and concentrated under reduced pressure. The residue was purified by silica gel flash chromatography (Eluent of 0˜6% MeOH in CH₂Cl₂) to give tert-butyl (1-(4-(2-(2-aminopyridin-3-yl)-5-phenyl-3H-imidazo[4,5-b]pyridin-3-yl)benzyl)piperidin-4-yl)(methyl-d₃)carbamate (750 mg, yield: 49%) as a yellow solid. MS: m/z=593.3 [M+H]⁺.

### Step 5: 3-(3-(4-((4-((Methyl-d₃)amino)piperidin-1-yl)methyl)phenyl)-5-phenyl-3H-imidazo[4,5-b]pyridin-2-yl)pyridin-2-amine

To a solution of tert-butyl (1-(4-(2-(2-aminopyridin-3-yl)-5-phenyl-3H-imidazo[4,5-b]pyridin-3-yl)benzyl)piperidin-4-yl)(methyl-d₃)carbamate (430 mg, 725 μmol) in CH₂Cl₂ (5 mL) was added TFA (165 mg, 1.45 mmol). The mixture was stirred at 25° C. for 1 hr. The mixture was diluted with H₂O (10 mL), and pH was adjusted to about 8 by NaHCO₃ (aq.). The mixture was extracted with CH₂Cl₂ (10 mL×2). The combined organic layers were washed with brine (10 mL×2), dried over Na₂SO₄, filtered and concentrated under reduced pressure. After purified by prep-TLC (CH₂Cl₂:MeOH=5: 1), 3-(3-(4-((4-((methyl-d₃)amino)piperidin-1-yl)methyl)phenyl)-5-phenyl-3H-imidazo[4,5-b]pyridin-2-yl)pyridin-2-amine (Intermediate 41, 350 mg crude, yield: 50%) was obtained as a light-yellow solid. MS: m/z=493.3 [M+H]+. ¹H NMR (400 MHz, Methanol-d₄) δ8.19 (d, J=8.4 Hz, 1H), 8.03 (d, J=7.2 Hz, 2H), 7.98 (dd, J=4.8, 1.6 Hz, 1H), 7.93 (d, J=8.4 Hz, 1H), 7.53 (d, J=8.4 Hz, 2H), 7.47-7.35 (m, 5H), 7.32 (dd, J=7.6, 1.6 Hz, 1H), 6.47 (dd, J=7.6, 4.8 Hz, 1H), 3.65 (s, 2H), 3.08-3.03 (m, 2H), 3.02-2.95 (m, 1H), 2.21-2.15 (m, 2H), 2.08-2.05 (m, 2H), 1.67-1.61 (m, 2H).

### Intermediate 42: tert-Butyl 4-(4-(2-(2-aminopyridin-3-yl)-5-chloro-3H-imidazo[4,5-b]pyridin-3-yl)benzyl)piperazine-1-carboxylate

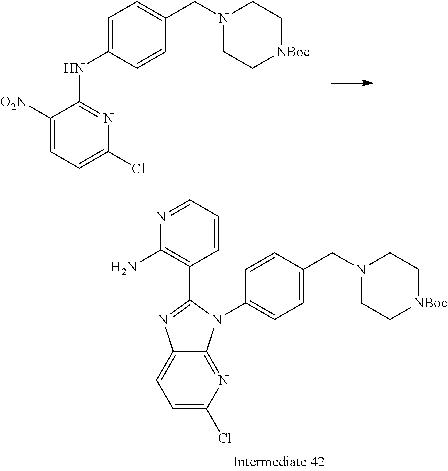 [SMILES: CC(C)(C)OC(=O)N1CCN(Cc2ccc(Nc3nc(Cl)ccc3[N+](=O)[O-])cc2)CC1 ; CC(C)(C)OC(=O)N1CCN(Cc2ccc(-n3c(-c4cccnc4N)nc4ccc(Cl)nc43)cc2)CC1]

To a solution of tert-butyl 4-(4-((6-chloro-3-nitropyridin-2-yl)amino)benzyl)piperazine-1-carboxylate (refer to Intermediate 10 for detail procedures, 21 g, 46.9 mmol) in DMSO (300 mL) were added 2-aminonicotinaldehyde (6.87 g, 56.4 mmol) and Na₂S₂O₄ (28.8 g, 141 mmol). The mixture was stirred at 100° C. for 16 hr. The reaction mixture was quenched with H₂O (300 mL) at 25° C., and extracted with CH₂Cl₂ (500 mL×2). The combined organic layers were washed with H₂O (1000 mL×3), dried over Na₂SO₄, filtered and concentrated under reduced pressure to give a crude product. After purified by silica gel flash chromatography (Eluent of 1˜8% MeOH in CH₂Cl₂), tert-butyl 4-(4-(2-(2-aminopyridin-3-yl)-5-chloro-3H-imidazo[4,5-b]pyridin-3-yl)benzyl)piperazine-1-carboxylate (Intermediate 42, 6.3 g, yield: 25%) was obtained as a yellow solid. MS: m/z=520.1 [M+H]⁺. ¹H NMR (400 MHz, Chloroform-d) δ 8.05 (dd, J=4.8, 2.0 Hz, 1H), 8.02 (d, J=8.4 Hz, 1H), 7.49 (d, J=8.4 Hz, 2H), 7.31 (dd, J=8.4, 2.8 Hz, 3H), 7.02 (dd, J=8.0, 2.0 Hz, 1H), 6.62 (br s, 2H), 6.33 (dd, J=8.0, 4.8 Hz, 1H), 3.61 (s, 2H), 3.50-3.45 (m, 4H), 2.49-2.43 (m, 4H), 1.47 (s, 9H).

### Intermediate 43: 3-(5-(4-Chlorophenyl)-3-(4-(piperazin-1-ylmethyl)phenyl)-3H-imidazo[4,5-b]pyridin-2-yl)pyridin-2-amine hydrochloride

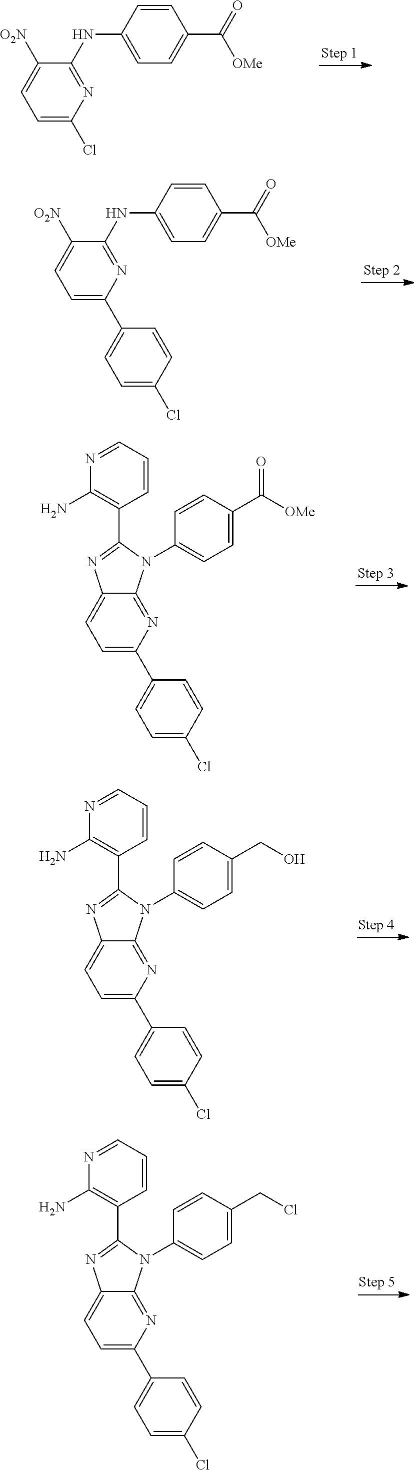 [SMILES: COC(=O)c1ccc(Nc2nc(Cl)ccc2[N+](=O)[O-])cc1 ; COC(=O)c1ccc(Nc2nc(-c3ccc(Cl)cc3)ccc2[N+](=O)[O-])cc1 ; COC(=O)c1ccc(-n2c(-c3cccnc3N)nc3ccc(-c4ccc(Cl)cc4)nc32)cc1 ; Nc1ncccc1-c1nc2ccc(-c3ccc(Cl)cc3)nc2n1-c1ccc(CO)cc1 ; Nc1ncccc1-c1nc2ccc(-c3ccc(Cl)cc3)nc2n1-c1ccc(CCl)cc1]

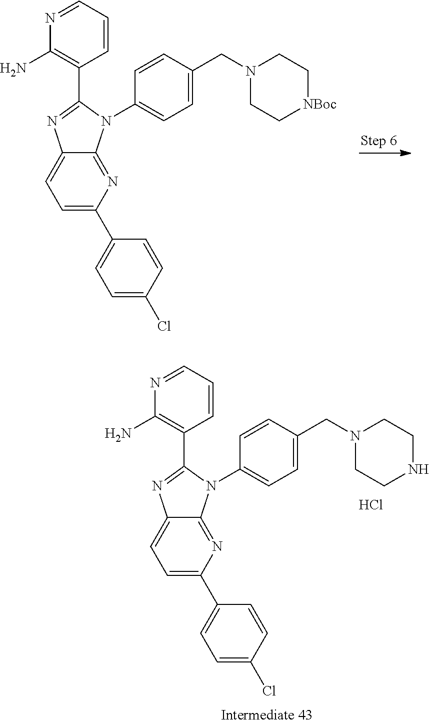

### Step 1: Methyl 4-((6-(4-chlorophenyl)-3-nitropyridin-2-yl)amino)benzoate

A mixture of methyl 4-((6-chloro-3-nitropyridin-2-yl)amino)benzoate (refer to Intermediate 13 for detail procedures, 5 g, 16.3 mmol), (4-chlorophenyl)boronic acid (2.54 g, 16.3 mmol), Cs₂CO₃ (15.9 g, 48.8 mmol), and Pd(dppf)Cl₂ (2.38 g, 3.25 mmol) in 1,4-dioxane (100 mL) and H₂O (20 mL) was degassed and purged with N₂ three times, and then the mixture was stirred at 80° C. for 16 hr under N₂ atmosphere. The reaction mixture was diluted with H₂O (100 mL) and extracted with CH₂Cl₂ (50 mL×4). The combined organic layers were washed with brine (30 mL×3), dried over Na₂SO₄, filtered and concentrated under reduced pressure. The residue was purified by silica gel flash chromatography (Eluent of 35 ˜45% EtOAc in petroleum ether) to give methyl 4-((6-(4-chlorophenyl)-3-nitropyridin-2-yl)amino)benzoate (5 g, yield: 80%) as a yellow solid. MS: m/z=383.9 [M+H]⁺.

### Step 2: Methyl 4-(2-(2-aminopyridin-3-yl)-5-(4-chlorophenyl)-3H-imidazo[4,5-b]pyridin-3-yl)benzoate

To a solution of methyl 4-((6-(4-chlorophenyl)-3-nitropyridin-2-yl)amino)benzoate (5 g, 13.0 mmol) and 2-aminonicotinaldehyde (1.75 g, 14.3 mmol) in DMSO (150 mL) was added Na₂S₂O₄ (9.07 g, 52.1 mmol). The mixture was stirred at 100° C. for 16 hr. The reaction mixture was diluted with H₂O (100 mL) and extracted with CH₂Cl₂ (500 mL×6). The combined organic layers were washed with brine (250 mL×2), dried over Na₂SO₄, filtered and concentrated under reduced pressure. The residue was purified by silica gel flash chromatography (Eluent of 25˜30% EtOAc in CH₂Cl₂) to give methyl 4-(2-(2-aminopyridin-3-yl)-5-(4-chlorophenyl)-3H-imidazo[4,5-h]pyridin-3-yl)benzoate (2.3 g, yield. 34%) as a yellow solid. MS: m/z=456.0 [M+H]⁺.

### Step 3: (4-(2-(2-Aminopyridin-3-yl)-5-(4-chlorophenyl)-3H-imidazo[4,5-b]pyridin-3-yl)phenyl)methanol

To a solution of 4-(2-(2-aminopyridin-3-yl)-5-(4-chlorophenyl)-3H-imidazo[4,5-b]pyridin-3-yl)benzoate (2.3 g, 5.05 mmol) in THF (30 mL) was added LiAlH₄ (2.5 M, 2.42 mL) at 0° C., then the mixture was stirred at 25° C. for 2 hr. The reaction mixture was quenched with Na₂SO₄—H₂O (4 g) at 0° C., and the mixture was filtered, the filter cake was washed by CH₂Cl₂ (30 mL×3). The filtrate was concentrated under reduced pressure to give (4-(2-(2-aminopyridin-3-yl)-5-(4-chlorophenyl)-3H-imidazo[4,5-b]pyridin-3-yl)phenyl)methanol (2.1 g, yield: 97%) as a yellow solid, which was directly used in the next step without purification. MS: m/z=428.1 [M+H]⁺.

### Step 4: 3-(3-(4-(Chloromethyl)phenyl)-5-(4-chlorophenyl)-3H-imidazo[4,5-b]pyridin-2-yl)pyridin-2-amine

To a solution of (4-(2-(2-aminopyridin-3-yl)-5-(4-chlorophenyl)-3H-imidazo[4,5-b]pyridin-3-yl)phenyl)methanol (1.85 g, 4.32 mmol) in CH₂Cl₂ (40 mL) was added SOCl₂ (1.54 g, 13.0 mmol) at 0° C. The mixture was stirred at 40° C. for 3 hr. The reaction mixture was quenched with H₂O (5 mL) at 0° C., and then filtered and concentrated under reduced pressure to give 3-(3-(4-(chloromethyl)phenyl)-5-(4-chlorophenyl)-3H-imidazo[4,5-b]pyridin-2-yl)pyridin-2-amine (1.9 g, yield: 98%) as a black brown solid. MS: m/z=455.9, 447.8 [M+H]⁺.

### Step 5: tert-Butyl 4-(4-(2-(2-aminopyridin-3-yl)-5-(4-chlorophenyl)-3H-imidazo[4,5-b]pyridin-3-yl)benzyl)piperazine-1-carboxylate

To a solution of 3-(3-(4-(chloromethyl)phenyl)-5-(4-chlorophenyl)-3H-imidazo[4,5-b]pyridin-2-yl)pyridin-2-amine (1 g, 2.24 mmol), tert-butyl piperazine-1-carboxylate (417 mg, 2.24 mmol) in DMF (10 mL) were added NaI (67.2 mg, 448 μmol) and K₂CO₃ (929 mg, 6.72 mmol). The mixture was stirred at 25° C. for 16 hr. The residue was diluted with H₂O (30 mL) and extracted with EtOAc (30 mL×3). The combined organic layers were washed with brine (20 mL×2), dried over Na₂SO₄, filtered and concentrated under reduced pressure. The residue was purified by silica gel flash chromatography (Eluent of 0˜4% MeOH in CH₂Cl₂) to give tert-butyl 4-(4-(2-(2-aminopyridin-3-yl)-5-(4-chlorophenyl)-3H-imidazo[4,5-b]pyridin-3-yl)benzyl)piperazine-1-carboxylate (550 mg, yield: 40%) as a yellow solid. MS: m/z=596.1 [M+H]⁺.

### Step 6: 3-(5-(4-Chlorophenyl)-3-(4-(piperazin-1-ylmethyl)phenyl)-3H-imidazo[4,5-b]pyridin-2-yl)pyridin-2-amine

To a solution of tert-butyl 4-(4-(2-(2-aminopyridin-3-yl)-5-(4-chlorophenyl)-3H-imidazo[4,5-b]pyridin-3-yl)benzyl)piperazine-1-carboxylate (550 mg, 923 μmol) in 1,4-dioxane (5 mL) was added 4.0 M HCl in 1,4-dioxane (3 mL) at 25° C. The mixture was stirred at 25° C. for 1 hr. The reaction was concentrated under reduced pressure to give 3-(5-(4-chlorophenyl)-3-(4-(piperazin-1-ylmethyl)phenyl)-3H-imidazo[4,5-b]pyridin-2-yl)pyridin-2-amine (Intermediate 43, 550 mg, HCl salt, yield: 98%) as a yellow solid. MS: m/z=496.0 [M+H]+. ¹H NMR (400 MHz, Methanol-d₄) δ 8.33 (d, J=8.4 Hz, 1H), 8.08-8.02 (m, 4H), 7.95 (d, J=8.4 Hz, 2H), 7.90 (d, J=7.6 Hz, 1H), 7.74 (d, 1=8.0 Hz, 2H), 7.45 (d, J=8.8 Hz, 2H), 6.94 (t, 1=7.2 Hz, 1H), 4.67 (s, 2H), 3.80-3.64 (m, 8H).

### Intermediate 44: tert-Butyl (1-(4-(2-(2-aminopyridin-3-yl)-5-chloro-3H-imidazo[4,5-b]pyridin-3-yl)benzyl)piperidin-4-yl)carbamate

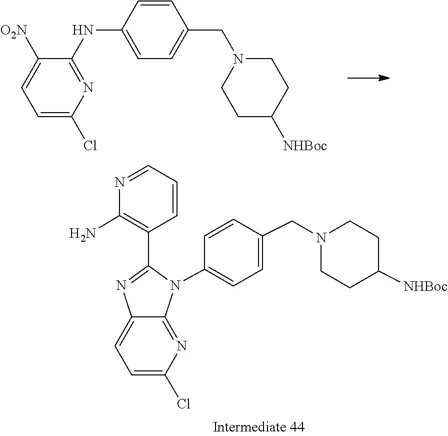 [SMILES: CC(C)(C)OC(=O)NC1CCN(Cc2ccc(Nc3nc(Cl)ccc3[N+](=O)[O-])cc2)CC1 ; CC(C)(C)OC(=O)NC1CCN(Cc2ccc(-n3c(-c4cccnc4N)nc4ccc(Cl)nc43)cc2)CC1]

A mixture of tert-butyl (1-(4-((6-chloro-3-nitropyridin-2-yl)amino)benzyl)piperidin-4-yl)carbamate (refer to Intermediate 11 for detail procedures, 13.0 g, 28.0 mmol), 2-aminopyridine-3-carbaldehyde (4.12 g, 33.8 mmol), and Na₂S₂O₄ (23.1 g, 112 mmol) in DMSO (500 mL) was degassed and purged with N₂ three times, and then the mixture was stirred at 100° C. for 16 hr under N₂ atmosphere. The reaction mixture was diluted with water (500 mL) and extracted with EtOAc (500 mL×3). The combined organic layers were dried over Na₂SO₄, filtered and concentrated under reduced pressure. The residue was purified by silica gel flash chromatography (Eluent of 0˜8% MeOH in CH₂Cl₂) to give tert-butyl (1-(4-(2-(2-aminopyridin-3-yl)-5-chloro-3H-imidazo[4,5-b]pyridin-3-yl)benzyl)piperidin-4-yl)carbamate (Intermediate 44, 4.8 g, yield: 28%) as a yellow solid. MS: m/z=534.3 [M+H]⁺.

### Intermediate 45: 3-(2-(2-Aminopyridin-3-yl)-3-(4-(piperazin-1-ylmethyl)phenyl)-3H-imidazo[4,5-b]pyridin-5-yl)pyridin-2(1H)-one

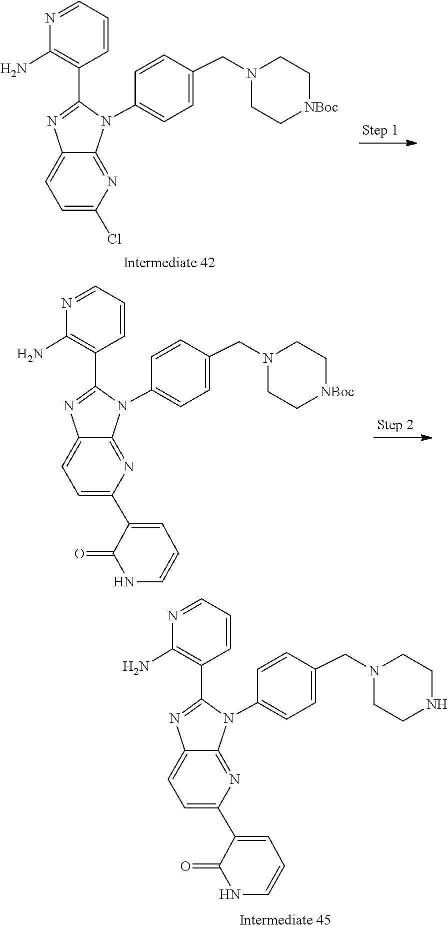

### Step 1: tert-Butyl 4-(4-(2-(2-aminopyridin-3-yl)-5-(2-oxo-1,2-dihydropyridin-3-yl)-3H-imidazo[4,5-b]pyridin-3-yl)benzyl)piperazine-1-carboxylate

A mixture of Intermediate 42 (250 mg, 481 μmol), (2-oxo-1,2-dihydropyridin-3-yl)boronic acid (80.1 mg, 577 μmol), Cs₂CO₃ (470 mg, 1.44 mmol), and Pd(dppf)Cl₂ (70.4 mg, 96.2 μmol) in 1,4-dioxane (5 mL) and H₂O (1 mL) was degassed and purged with N₂ three times, and then the mixture was stirred at 80° C. for 16 hr under N₂ atmosphere. The reaction mixture was filtered at 25° C., and then diluted with H₂O (20 mL) and extracted with EtOAc (20 mL×3). The combined organic layers were washed with brine (30 mL), dried over Na₂SO₄, filtered, and concentrated under reduced pressure. After purified by silica gel flash chromatography (Eluent of 1˜100% EtOAc in petroleum ether and eluent of 1˜10% MeOH in CH₂Cl₂), tert-butyl 4-(4-(2-(2-aminopyridin-3-yl)-5-(2-oxo-1,2-dihydropyridin-3-yl)-3H-imidazo[4,5-b]pyridin-3-yl)benzyl)piperazine-1-carboxylate (200 mg, yield: 58%) was obtained as a brown solid. MS: m/z=579.3 [M+H]⁺.

### Step 2: 3-(2-(2-Aminopyridin-3-yl)-3-(4-(piperazin-1-ylmethyl)phenyl)-3H-imidazo[4,5-b]pyridin-5-yl)pyridin-2(1H)-one

To a solution of tert-butyl 4-(4-(2-(2-aminopyridin-3-yl)-5-(2-oxo-1,2-dihydropyridin-3-yl)-3H-imidazo[4,5-b]pyridin-3-yl)benzyl)piperazine-1-carboxylate (200 mg, 346 μmol) in 1,4-dioxane (3 mL) was added 4 M HCl in 1,4-dioxane (3 mL) at 25° C. The mixture was stirred at 25° C. for 16 hr. The pH of the mixture was adjusted to ˜8 with NaHCO₃. Then the mixture was extracted with CH₂Cl₂ (20 mL×3). The combined organic layers were washed with brine (20 mL), dried over Na₂SO₄, filtered and concentrated under reduced pressure to give 3-(2-(2-aminopyridin-3-yl)-3-(4-(piperazin-1-ylmethyl)phenyl)-3H-imidazo[4,5-b]pyridin-5-yl)pyridin-2(1H)-one (Intermediate 45, 200 mg, yield: 85%) as a yellow solid. MS: m/z=479.2 [M+H]⁺.

### Intermediate 46: 3-(5-Morpholino-3-(4-(piperazin-1-ylmethyl)phenyl)-3H-imidazo[4,5-b]pyridin-2-yl)pyridin-2-amine

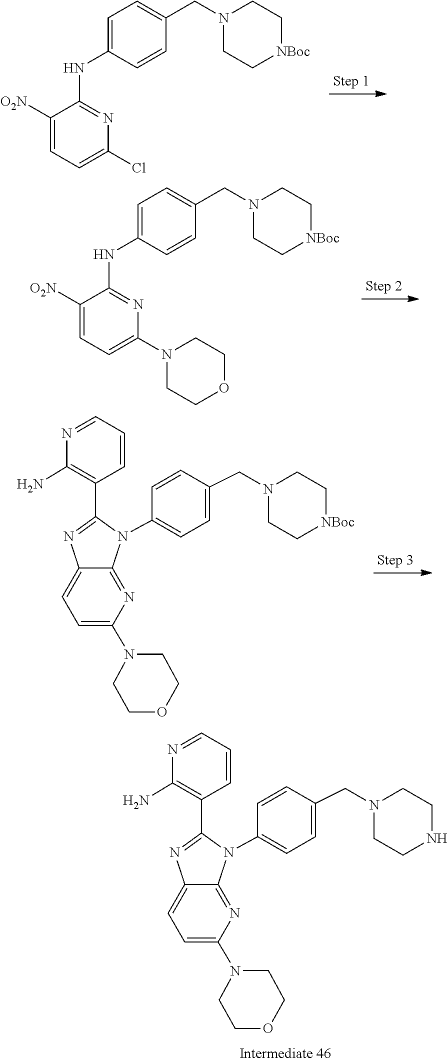

### Step 1: Tert-butyl 4-(4-((6-morpholino-3-nitropyridin-2-yl)amino)benzyl)piperazine-1-carboxylate

To a solution of tert-butyl 4-(4-((6-chloro-3-nitropyridin-2-yl)amino)benzyl)piperazine-1-carboxylate (refer to Intermediate 10 for detail procedures, 300 mg, 670 μmol) and morpholine (117 mg, 1.34 mmol) in CH₃CN (4 mL) was added DIEA (173 mg, 1.34 mmol). The mixture was stirred at 90° C. for 2 hr. The reaction mixture was quenched with H₂O (10 mL) at 25° C., and then extracted with CH₂Cl₂ (15 mL×2). The combined organic layers were washed with brine (15 mL), dried over Na₂SO₄, filtered and concentrated under reduced pressure to give tert-butyl 4-(4-((6-morpholino-3-nitropyridin-2-yl)amino)benzyl)piperazine-1-carboxylate (330 mg, yield: 93%) as a yellow solid. MS: m/z=499.2 [M+H]⁺. ¹H NMR (400 MHz, Chloroform-d) δ 10.71 (s, 1H), 8.32 (d, J=9.2 Hz, 1H), 7.53 (d, J=8.0 Hz, 2H), 7.30 (d, J=8.0 Hz, 2H), 6.13 (d, J=9.6 Hz, 1H), 3.79-3.75 (m, 4H), 3.73-3.67 (m, 4H), 3.50 (s, 2H), 3.46-3.40 (m, 4H), 2.44-2.35 (m, 4H), 1.45 (s, 9H).

### Step 2: tert-Butyl 4-(4-(2-(2-aminopyridin-3-yl)-5-morpholino-3H-imidazo[4,5-b]pyridin-3-yl)benzyl)piperazine-1-carboxylate

To a solution of tert-butyl 4-(4-((6-morpholino-3-nitropyridin-2-yl)amino)benzyl)piperazine-1-carboxylate (330 mg, 662 μmol) in DMSO (10 mL) were added 2-aminonicotinaldehyde (97 mg, 794 μmol) and Na₂S₂O₄ (542 mg, 2.65 mmol). The mixture was stirred at 100° C. for 2 hr. The reaction mixture was quenched with H₂O (20 mL) at 25° C., and extracted with CH₂Cl₂ (50 mL×3). The combined organic layers were washed with brine (50 mL), dried over Na₂SO₄, filtered and concentrated under reduced pressure to give the crude product. The crude was purified by silica gel flash chromatography (Eluent of 1˜5% MeOH in CH₂Cl₂) to give tert-butyl 4-(4-(2-(2-aminopyridin-3-yl)-5-morpholino-3H-imidazo[4,5-h]pyridin-3-yl)benzyl)piperazine-1-carboxylate (270 mg, yield: 68%) as a yellow solid. MS: m/z=571.2 [M+H]⁺.

### Step 3: 3-(5-Morpholino-3-(4-(piperazin-1-ylmethyl)phenyl)-3H-imidazo[4,5-b]pyridin-2-yl)pyridin-2-amine

To a solution of ter-butyl 4-(4-(2-(2-aminopyridin-3-yl)-5-morpholino-3H-imidazo[4,5-b]pyridin-3-yl)benzyl)piperazine-1-carboxylate (270 mg, 473 μmol) in 1,4-dioxane (2 mL) was added 4 M HCl in 1,4-dioxane (2 mL) at 25° C. The mixture was stirred at 25° C. for 16 hr. The reaction was concentrated under reduced pressure to give 3-(5-morpholino-3-(4-(piperazin-1-ylmethyl)phenyl)-3H-imidazo[4,5-b]pyridin-2-yl)pyridin-2-amine (Intermediate 46, 240 mg, HCl salt, yield: 93%) as a yellow solid. MS: m/z=471.2 [M+H]⁺. ¹H NMR (400 MHz, Methanol-d₄) δ 8.13-7.99 (m, 2H), 7.96-7.82 (m, 3H), 7.66 (dd, J=8.4, 3.2 Hz, 2H), 7.14-7.02 (m, 1H), 7.01-6.90 (m, 1H), 4.66-4.59 (m, 2H), 3.82-3.77 (m, 4H), 3.73-3.69 (m, 6H), 3.68 (s, 2H), 3.60-3.55 (m, 4H).

### Intermediate 47: 6-(2-(2-Aminopyridin-3-yl)-3-(4-(piperazin-1-ylmethyl)phenyl)-3H-imidazo[4,5-b]pyridin-5-yl)pyridin-2(1H)-one

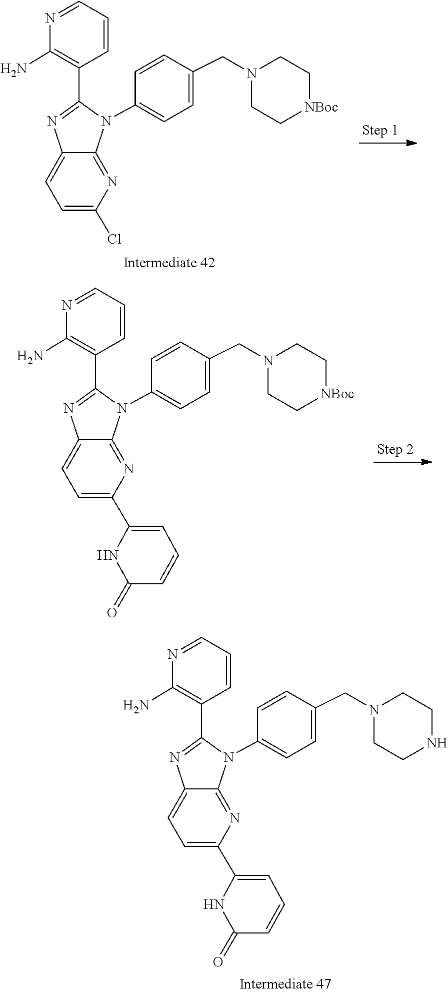 [SMILES: CC(C)(C)OC(=O)N1CCN(Cc2ccc(-n3c(-c4cccnc4N)nc4ccc(Cl)nc43)cc2)CC1 ; CC(C)(C)OC(=O)N1CCN(Cc2ccc(-n3c(-c4cccnc4N)nc4ccc(-c5cccc(=O)[nH]5)nc43)cc2)CC1 ; Nc1ncccc1-c1nc2ccc(-c3cccc(=O)[nH]3)nc2n1-c1ccc(CN2CCNCC2)cc1]

### Step 1: Tert-butyl 4-(4-(2-(2-aminopyridin-3-yl)-5-(6-oxo-1,6-dihydropyridin-2-yl)-3H-imidazo[4,5-b]pyridin-3-yl)benzyl)piperazine-1-carboxylate

A mixture of Intermediate 42 (500 mg, 961 μmol), 6-(4,4,5,5-tetramethyl-1,3,2-dioxaborolan-2-yl)pyridin-2(1H)-one (255 mg, 1.15 mmol), Cs₂CO₃ (940 mg, 2.88 mmol), and Pd(dppf)Cl₂ (141 mg, 192 μmol) in 1,4-dioxane (10 mL) and H₂O (2 mL) was degassed and purged with N₂ three times, and then the mixture was stirred at 80° C. for 16 hr under N₂ atmosphere. The reaction mixture was filtered through Celite at 25° C., and then diluted with H₂O (20 mL) and extracted with EtOAc (20 mL×3). The combined organic layers were washed with brine (30 mL), dried over Na₂SO₄, filtered, and concentrated under reduced pressure. After purified by silica gel flash chromatography (Eluent of 1˜6% MeOH in CH₂Cl₂), tert-butyl 4-(4-(2-(2-aminopyridin-3-yl)-5-(6-oxo-1,6-dihydropyridin-2-yl)-3H-imidazo[4,5-b]pyridin-3-yl)benzyl)piperazine-1-carboxylate (500 mg, yield: 76%) was obtained as a brown solid. MS: m/z=579.2 [M+H]⁺.

### Step 2: 6-(2-(2-Aminopyridin-3-yl)-3-(4-(piperazin-1-ylmethyl)phenyl)-3H-imidazo[4,5-b]pyridin-5-yl)pyridin-2(1H)-one

To a solution of tert-butyl 4-(4-(2-(2-aminopyridin-3-yl)-5-(6-oxo-1,6-dihydropyridin-2-yl)-3H-imidazo[4,5-b]pyridin-3-yl)benzyl)piperazine-1-carboxylate (150 mg, 259 μmol) in 1,4-dioxane (2 mL) was added 4 M HCl in 1,4-dioxane (2 mL) at 25° C. The mixture was stirred at 25° C. for 16 hr. The reaction was concentrated under reduced pressure to give the crude (HCl salt). The crude was diluted with aqueous NaHCO₃ (10 mL) and extracted with CH₂Cl₂ (10 mL×5). The combined organic layers were washed with brine (20 mL), dried over Na₂SO₄, filtered and concentrated under reduced pressure to give 6-(2-(2-aminopyridin-3-yl)-3-(4-(piperazin-1-ylmethyl)phenyl)-3H-imidazo[4,5-b]pyridin-5-yl)pyridin-2(1H)-one (Intermediate 47, 68.2 mg, yield: 52%) as a yellow solid. MS: m/z=479.2 [M+H]⁺. ¹H NMR (400 MHz, Dimethylsulfoxide-d₆) δ 8.33 (d, J=8.4 Hz, 1H), 8.16 (d, J=8.4 Hz, 1H), 8.01 (dd, 1=4.8, 2.0 Hz, 1H), 7.60 (dd, 1=8.8, 6.8 Hz, 1H), 7.52-7.41 (m, 5H), 7.20-7.14 (m, 2H), 7.02 (br s, 2H), 6.46 (d, J=8.8 Hz, 1H), 6.38 (dd, J=7.6, 4.8 Hz, 1H), 3.53 (s, 2H), 2.73-2.69 (m, 4H), 2.36-2.30 (m, 4H).

### Intermediate 48: 3-(3-(4-((4-Aminopiperidin-1-yl)methyl)phenyl)-5-morpholino-3H-imidazo[4,5-b]pyridin-2-yl)pyridin-2-amine

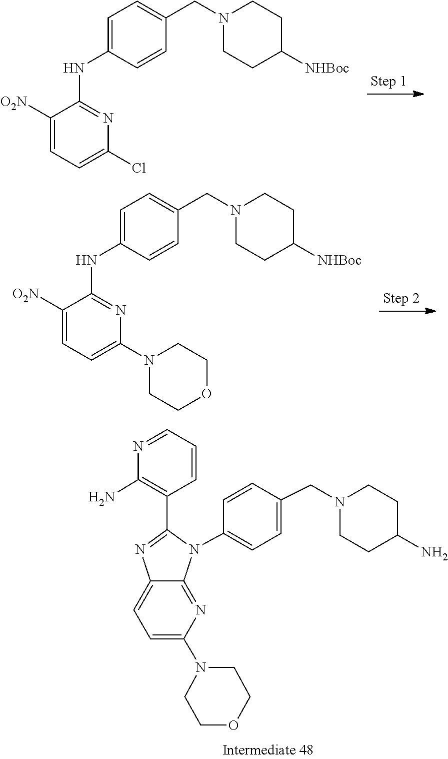

### Step 1: tert-Butyl (1-(4-((6-morpholino-3-nitropyridin-2-yl)amino)benzyl)piperidin-4-yl)carbamate

A mixture of tert-butyl (1-(4-((6-chloro-3-nitropyridin-2-yl)amino)benzyl)piperidin-4-yl)carbamate (refer to Intermediate 11 for detail procedures, 3 g, 6.49 mmol), morpholine (678 mg, 7.79 mmol), DIEA (1.68 g, 12.9 mmol) in MeCN (40 mL) was stirred at 90° C. for 2 hr. The reaction mixture was diluted with H₂O (100 mL) and extracted with CH₂Cl₂ (100 mL×3). The combined organic layers were washed with H₂O (50 mL×2), dried over Na₂SO₄, filtered and concentrated to give tert-butyl (1-(4-((6-morpholino-3-nitropyridin-2-yl)amino)benzyl)piperidin-4-yl)carbamate (3.24 g, yield: 97%) as a yellow solid, which was directly used in the next step. MS: m/z=513.2 [M+H]⁺. ¹H NMR (400 MHz, Chloroform-d) 10.70 (s, 1H), 8.32 (d, J=9.2 Hz, 1H), 7.52 (d, J=8.4 Hz, 2H), 7.30 (d, J=8.0 Hz, 2H), 6.12 (d, J=9.6 Hz, 1H), 4.53-4.34 (m, 1H), 3.79-3.75 (m, 4H), 3.75-3.55 (m, 4H), 3.48 (s, 3H), 2.90-2.75 (m, 2H), 2.15-2.05 (m, 2H), 1.95-1.75 (m, 2H), 1.76-1.53 (m, 2H), 1.44 (s, 9H).

### Step 2: 3-(3-(4-((4-Aminopiperidin-1-yl)methyl)phenyl)-5-morpholino-3H-imidazo[4,5-b]pyridin-2-yl)pyridin-2-amine

A mixture of tert-butyl (1-(4-((6-morpholino-3-nitropyridin-2-yl)amino)benzyl)piperidin-4-yl)carbamate (3.24 g, 6.32 mmol), 2-aminopyridine-3-carbaldehyde (926 mg, 7.58 mmol), and Na₂S₂O₄ (3.88 g, 18.9 mmol) in DMSO (90 mL) was stirred at 100° C. for 24 hr. The reaction mixture was diluted with H₂O (200 mL) and extracted with CH₂Cl₂ (50 mL×3). Then sat. aq. Na₂CO₃ (100 mL) was added to aqueous phase to adjust pH about 8-9. The aqueous phase was extracted with EtOAc (50 mL×6), dried over Na₂SO₄, filtered and concentrated under reduced pressure. The residue was purified by silica gel flash chromatography (Eluent of 0˜7% MeOH in CH₂Cl₂) to give 3-(3-(4-((4-aminopiperidin-1-yl)methyl)phenyl)-5-morpholino-3H-imidazo[4,5-b]pyridin-2-yl)pyridin-2-amine (1.2 g, yield: 29%) as a yellow solid. The product (80 mg) was purified by prep-HPLC (column. Phenomenex C18 150*25 mm*10 μm; mobile phase: [water (NH₄HCO₃)-ACN]; gradient: 7%-37% B over 10 min) to give 3-(3-(4-((4-aminopiperidin-1-yl)methyl)phenyl)-5-morpholino-3H-imidazo[4,5-b]pyridin-2-yl)pyridin-2-amine (Intermediate 48, 36.5 mg) as a light-yellow powder. MS: m/z=485.3 [M+H]˜. ¹H NMR (400 MHz, Dimethylsulfoxide-d₆) δ 7.98 (d, J=8.8 Hz, 1H), 7.96 (dd, J=4.8, 2.0 Hz, 1H), 7.40 (d, J=8.4 Hz, 2H), 7.29 (d, J=8.4 Hz, 2H), 7.03 (br s, 2H), 6.98 (dd, J=8.0, 1.6 Hz, 1H), 6.89 (d, J=8.8 Hz, 1H), 6.30 (dd, J=8.0, 4.8 Hz, 1H), 3.69-3.65 (m, 4H), 3.50 (s, 2H), 3.41-3.38 (m, 5H), 2.78-2.70 (m, 2H), 2.02-1.92 (m, 2H), 1.70-1.64 (m, 2H), 1.35-1.16 (m, 2H).

### Intermediate 49: 3-(5-(4-fluorophenyl)-3-(4-(piperazin-1-ylmethyl)phenyl)-3H-imidazo[4,5-b]pyridin-2-yl)pyridin-2-amine hydrochloride

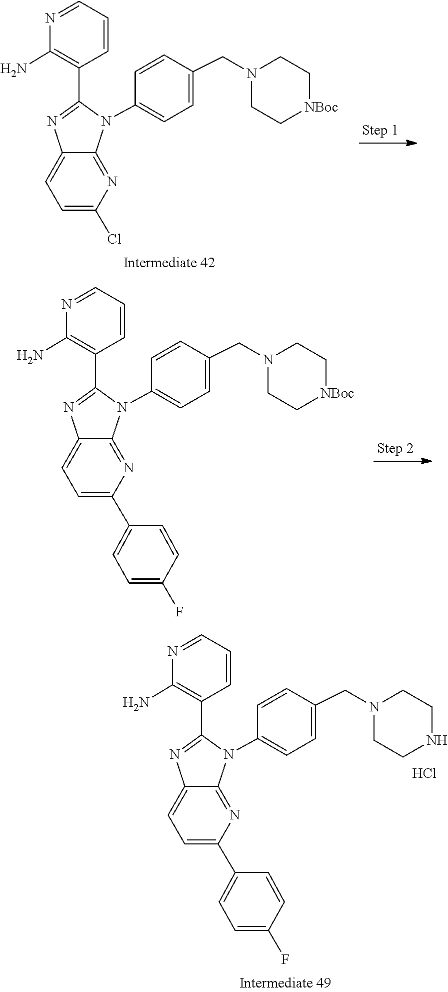

### Step 1: Tert-butyl 4-(4-(2-(2-aminopyridin-3-yl)-5-(4-fluorophenyl)-3H-imidazo[4,5-b]pyridin-3-yl)benzyl)piperazine-1-carboxylate

To a solution of Intermediate 42 (500 mg, 961 μmol) and (4-fluorophenyl)boronic acid (135 mg, 961 μmol) in 1,4-dioxane (10 mL) and H₂O (2 mL) were added Cs₂CO₃ (940 mg, 2.88 mmol) and Pd(dppf)Cl₂ (70.4 mg, 96.1 μmol). The mixture was degassed and purged with N₂ three times, the resulting mixture was stirred at 80° C. for 16 hr under N₂ atmosphere. Then the reaction mixture was diluted with H₂O (50 mL) and extracted with CH₂Cl₂ (50 mL×2). The combined organic layers were dried over Na₂SO₄, filtered and concentrated under reduced pressure. The crude product was purified by silica gel flash chromatography (Eluent of 50-100% EtOAc in petroleum ether) to give tert-butyl 4-(4-(2-(2-aminopyridin-3-yl)-5-(4-fluorophenyl)-3H-imidazo[4,5-b]pyridin-3-yl)benzyl)piperazine-1-carboxylate (500 mg, yield: 86%) as a yellow solid. MS: m/z=580.3 [M+H]⁺. ¹H NMR (400 MHz, Dimethylsulfoxide-d₆) δ 8.26 (d, J=8.4 Hz, 1H), 8.13-8.04 (m, 2H), 8.01-7.87 (m, 2H), 7.52-7.41 (m, 4H), 7.29 (t, J=8.8 Hz, 2H), 7.14 (dd, J=7.6, 1.6 Hz, 1H), 7.03 (s, 2H), 6.38 (dd, J=8.0, 4.8 Hz, 1H), 3.59 (s, 2H), 3.37-3.33 (m, 4H), 2.40-2.26 (m, 4H), 1.40 (s, 9H). ¹⁹F NMR (400 MHz, Dimethylsulfoxide-d₆) 6-113.59.

### Step 2: 3-(5-(4-fluorophenyl)-3-(4-(piperazin-1-ylmethyl)phenyl)-3H-imidazo[4,5-b]pyridin-2-yl)pyridin-2-amine hydrochloride

To a solution of tert-butyl 4-(4-(2-(2-aminopyridin-3-yl)-5-(4-fluorophenyl)-3H-imidazo[4,5-b]pyridin-3-yl)benzyl)piperazine-1-carboxylate (500 mg, 863 μmol) in 1,4-dioxane (5 mL) was added HCl/1,4-dioxane (4 M, 5 mL). The mixture was stirred at 25° C. for 0.5 hr. The reaction mixture was filtered and concentrated under reduced pressure to give 3-(5-(4-fluorophenyl)-3-(4-(piperazin-1-ylmethyl)phenyl)-3H-imidazo[4,5-b]pyridin-2-yl)pyridin-2-amine (Intermediate 49, 400 mg HCl salt, yield: 92%) as a yellow solid. MS: m/z=480.2 [M+H]˜. ¹H NMR (400 MHz, Dimethylsulfoxide-d₆) δ 13.00-12.05 (m, 1H), 10.26-9.46 (m, 2H), 8.60-8.48 (m, 1H), 8.38 (d, J=8.4 Hz, 1H), 8.19-8.06 (m, 4H), 7.90-7.82 (m, 3H), 7.68 (d, J=8.4 Hz, 2H), 7.32 (t, J=9.2 Hz, 2H), 7.03-6.93 (m, 1H), 4.51 (br s, 2H), 3.55-3.50 (m, 4H), 2.59-2.51 (m, 4H). ¹⁹F NMR (400 MHz, Dimethylsulfoxide-d₆) 6-113.04.

### Intermediate 50: 3-(3-(4-(Chloromethyl)phenyl)-5-cyclopropyl-3H-imidazo[4,5-b]pyridin-2-yl)pyridin-2-amine

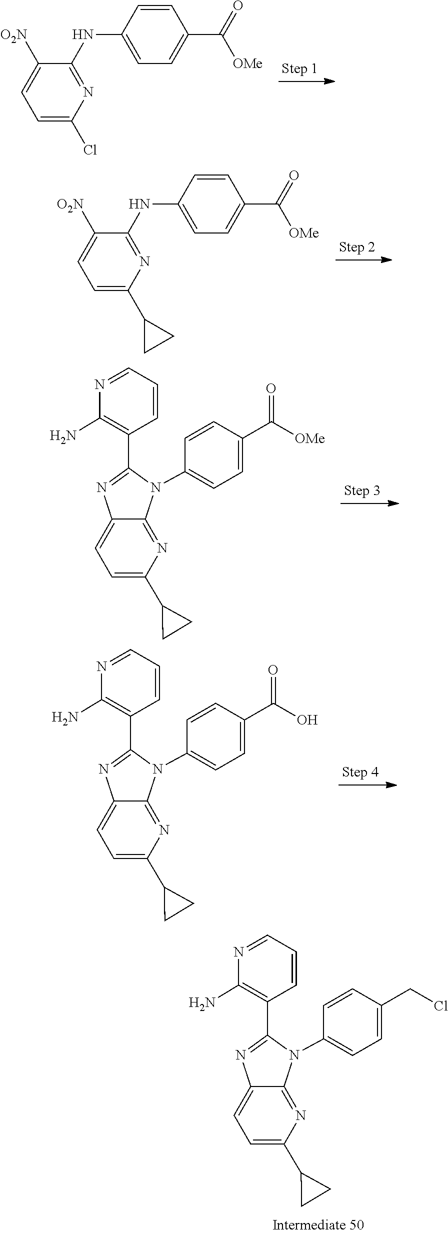

### Step 1: Methyl 4-((6-cyclopropyl-3-nitropyridin-2-yl)amino)benzoate

To a solution of methyl 4-((6-chloro-3-nitropyridin-2-yl)amino)benzoate (refer to Intermediate 13 for detail procedures, 30 g, 97.5 mmol) in 1,4-dioxane (300 mL) were added cyclopropylboronic acid (12.6 g, 146 mmol), Cs₂CO₃ (95.3 g, 292 mmol) and H₂O (50 mL) at 25° C. The mixture was stirred at 100° C. for 12 hr under N₂. The reaction mixture was poured into H₂O (500 mL) and extracted with CH₂Cl₂ (200 mL×2). The combined organic layers were washed with brine (400 mL), dried over anhydrous Na₂SO₄, filtered and concentrated. The residue was purified by silica gel flash chromatography (Eluent of 0˜50% EtOAc in petroleum ether, and then use CH₂Cl₂ directly) to give methyl 4-((6-cyclopropyl-3-nitropyridin-2-yl)amino)benzoate (25 g, yield: 74%) as an off-white solid. MS: m/z=314.0 [M+H]⁺. ¹H NMR (400 MHz, Dimethylsulfoxide-d₆) δ 10.20 (s, 1H), 8.42 (d, J=8.4 Hz, 1H), 7.94 (d, J=8.4 Hz, 2H), 7.80 (d, J=8.8 Hz, 2H), 7.05 (d, J=8.4 Hz, 1H), 3.84 (s, 3H), 2.28-2.18 (m, 1H), 1.15-1.08 (m, 2H), 1.07-1.01 (m, 2H).

### Step 2: Methyl 4-(2-(2-aminopyridin-3-yl)-5-cyclopropyl-3H-imidazo[4,5-b]pyridin-3-yl)benzoate

To a solution of methyl 4-((6-cyclopropyl-3-nitropyridin-2-yl)amino)benzoate (20 g, 63.8 mmol) in DMSO (500 mL) were added 2-aminopyridine-3-carbaldehyde (9.4 g, 76.6 mmol) and Na₂S₂O₄ (44.5 g, 255 mmol) at 25° C. The mixture was stirred at 100° C. for 12 hr. The reaction mixture was poured into H₂O (500 mL) and then extracted with CH₂Cl₂ (200 mL×2). The combined organic layers were washed with brine (400 mL), dried over anhydrous Na₂SO₄, filtered and concentrated to dryness. The residue was purified by silica gel flash chromatography (Eluent of 1˜3% MeOH in CH₂Cl₂) to give methyl 4-(2-(2-aminopyridin-3-yl)-5-cyclopropyl-3H-imidazo[4,5-b]pyridin-3-yl)benzoate (10 g, yield: 37%) as a red solid. MS: m/z=386.1 [M+H]⁺. ¹H NMR (400 MHz, Dimethylsulfoxide-d₆) δ 8.11-8.06 (m, 3H), 8.02 (dd, J=5.6, 1.6 Hz, 1H), 7.57 (d, J=8.4 Hz, 2H), 7.41-7.27 (m, 4H), 6.59 (dd, J=7.6, 5.2 Hz, 1H), 3.89 (s, 3H), 2.24-2.17 (m, 1H), 0.98-0.92 (m, 2H), 0.87-0.82 (m, 2H).

### Step 3: (4-(2-(2-Aminopyridin-3-yl)-5-cyclopropyl-3H-imidazo[4,5-b]pyridin-3-yl)phenyl)methanol

To a solution of methyl 4-(2-(2-aminopyridin-3-yl)-5-cyclopropyl-3H-imidazo[4,5-b]pyridin-3-yl)benzoate (2 g, 5.2 mmol) in THF (50 mL) was added LiAlH₄ (3.1 mL, 2.5 M) at 0° C. The mixture was stirred at 0° C. for 1.5 hr, and then was quenched with Na₂SO₄·10OH₂O (8 g) at 0° C. The mixture was filtered, and the filter cake was washed by CH₂Cl₂ (30 mL×2). The filtrate was concentrated under reduced pressure to give (4-(2-(2-aminopyridin-3-yl)-5-cyclopropyl-3H-imidazo[4,5-b]pyridin-3-yl)phenyl)methanol (1.8 g, crude) as a yellow solid, which was used in the next step without further purification. MS: m/z=358.0 [M]⁺.

### Step 4: 3-(3-(4-(Chloromethyl)phenyl)-5-cyclopropyl-3H-imidazo[4,5-b]pyridin-2-yl)pyridin-2-amine

To a solution of (4-(2-(2-aminopyridin-3-yl)-5-cyclopropyl-3H-imidazo[4,5-b]pyridin-3-yl)phenyl)methanol (1.4 g, 3.9 mmol) in CH₂Cl₂ (20 mL) was added SOCl₂ (2.8 g, 23.5 mmol) dropwise at 25° C. The mixture was stirred at 40° C. for 1 hr. The reaction mixture was concentrated to give 3-(3-(4-(chloromethyl)phenyl)-5-cyclopropyl-3H-imidazo[4,5-b]pyridin-2-yl)pyridin-2-amine (Intermediate 50, 1.43 g, yield: 93%) as an off-white solid. MS: m/z=376.0 [M+H]⁺. ¹H NMR (400 MHz, Dimethylsulfoxide-d₆) δ 8.59-8.24 (m, 2H), 8.17-8.08 (m, 2H), 7.81 (dd, J=7.2, 1.6 Hz, 1H), 7.60 (d, J=8.0 Hz, 2H), 7.48 (d, J=8.4 Hz, 2H), 7.35 (d, J=8.4 Hz, 1H), 6.87 (dd, J=7.6, 6.0 Hz, 1H), 4.85 (s, 2H), 2.27-2.17 (m, 1H), 1.00-0.92 (m, 2H), 0.88-0.81 (m, 2H).

### Intermediate 51: 4-(Piperidin-4-ylamino)pyrimidine-2-carbonitrile

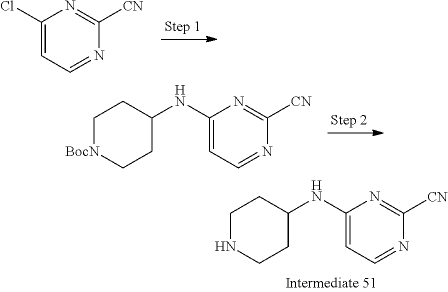

### Step 1: tert-Butyl 4-((2-cyanopyrimidin-4-yl)amino)piperidine-1-carboxylate

To a solution of tert-butyl 4-aminopiperidine-1-carboxylate (600 mg, 3.0 mmol) and 2-chloropyrimidine-4-carbonitrile (418 mg, 3.0 mmol) in DMF (5 mL) were added K₂CO₃ (1.24 g, 8.99 mmol) and NaI (89.8 mg, 599 μmol). The mixture was stirred at 80° C. for 1 hr. The reaction mixture was diluted with H₂O (20 mL), and then extracted with EtOAc (30 mL×2). The combined organic layers were washed with brine (50 mL), dried over Na₂SO₄, filtered, and concentrated under reduced pressure. The residue was purified by silica gel flash chromatography (Eluent of 0˜50% EtOAc in petroleum ether) to give tert-butyl 4-[(2-cyanopyrimidin-4-yl)amino]piperidine-1-carboxylate (860 mg, yield: 92%) as a white solid. ¹H NMR (400 MHz, Dimethylsulfoxide-d₆) δ 8.13-8.01 (m, 2H), 6.66 (d, J=5.6 Hz, 1H), 4.05-3.95 (m, 1H), 3.87-3.84 (m, 2H), 2.99-2.86 (m, 2H), 1.86-1.83 (m, 2H), 1.40 (s, 9H), 1.34-1.26 (m, 2H).

### Step 2: 4-(Piperidin-4-ylamino)pyrimidine-2-carbonitrile

To a solution of tert-butyl 4-[(2-cyanopyrimidin-4-yl)amino]piperidine-1-carboxylate (110 mg, 362 μmol) in DCM (3 mL) was added TFA (767 mg, 6.73 mmol). The mixture was stirred at 25° C. for 1 hr. The reaction mixture was concentrated under reduced pressure. The crude 4-(piperidin-4-ylamino)pyrimidine-2-carbonitrile (Intermediate 51, 120 mg, yield: 100%, TFA salt) was used in the next step without further purification. MS: m/z=204.0 [M+H]⁺.

### Intermediate 52: 6-(Piperidin-4-ylamino)pyrimidine-4-carbonitrile

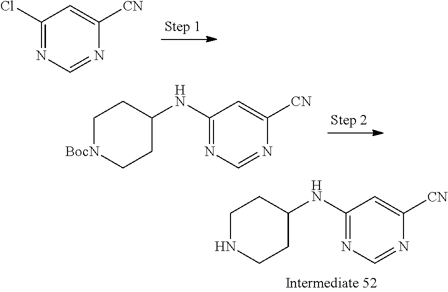

### Step 1: tert-Butyl 4-((6-cyanopyrimidin-4-yl)amino)piperidine-1-carboxylate)

To a solution of tert-butyl 4-aminopiperidine-1-carboxylate (700 mg, 3.50 mmol) and 6-chloropyrimidine-4-carbonitrile (487 mg, 3.50 mmol) in DMF (5 mL) were added K₂CO₃ (1.45 g, 10.5 mmol) and NaI (104 mg, 699 μmol). The mixture was stirred at 80° C. for 2 hr. The reaction mixture was diluted with H₂O (20 mL), and then extracted with EtOAc (30 mL×2). The combined organic layers were washed with brine (50 mL), dried over Na₂SO₄, filtered and concentrated under reduced pressure. The residue was purified by silica gel flash chromatography (Eluent of 0˜45% EtOAc in petroleum ether) to give tert-butyl 4-[(6-cyanopyrimidin-4-yl)amino]piperidine-1-carboxylate (1.0 g, yield: 94%) as a white solid. MS: m/z=304.0 [M+H]⁺. ¹H NMR (400 MHz, Dimethylsulfoxide-d₆) δ 8.50 (s, 1H), 8.07 (d, J=7.6 Hz, 1H), 6.92 (s, 1H), 4.08-4.01 (m, 1H), 3.88-3.85 (m, 2H), 2.95-2.75 (m, 2H), 1.86-1.8.3 (m, 2H), 1.40 (s, 9H), 1.34-1.27 (m, 2H).

### Step 2: 6-(Piperidin-4-ylamino)pyrimidine-4-carbonitrile

To a solution of tert-butyl 4-[(6-cyanopyrimidin-4-yl)amino]piperidine-1-carboxylate (285 mg, 939 μmol) in CH₂Cl₂ (5 mL) was added TFA (535 mg, 4.7 mol). The mixture was stirred at 25° C. for 1 hr. The reaction mixture was concentrated under reduced pressure. The crude product 6-(piperidin-4-ylamino)pyrimidine-4-carbonitrile (Intermediate 52, 298 mg TFA salt, yield: 100%), which was used in the next step without further purification. MS: m/z=204.0 [M+H]⁺.

### Intermediate 53: 4-(Piperazin-1-yl)pyrimidine-2-carbonitrile

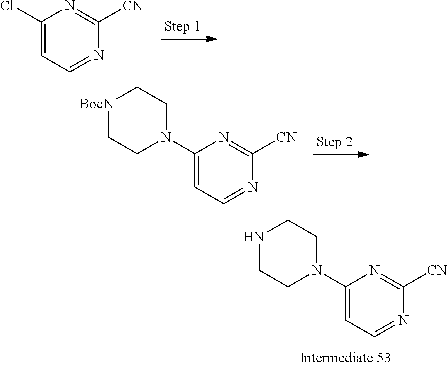 [SMILES: N#Cc1nccc(Cl)n1 ; CC(C)(C)OC(=O)N1CCN(c2ccnc(C#N)n2)CC1 ; N#Cc1nccc(N2CCNCC2)n1]

### Step 1: Tert-butyl 4-(2-cyanopyrimidin-4-yl)piperazine-1-carboxylate

To a solution of tert-butyl piperazine-1-carboxylate (550 mg, 2.95 mmol) and 2-chloropyrimidine-4-carbonitrile (412 mg, 2.95 mmol) in DMF (5 mL) were added K₂CO₃ (1.22 g, 8.86 mmol) and NaI (88.5 mg, 590 μmol). The mixture was stirred at 80° C. for 1 hr. The reaction mixture was diluted with H₂O (20 mL), and then extracted with EtOAc (30 mL×2). The combined organic layers were washed with brine (50 mL), dried over Na₂SO₄, filtered and concentrated under reduced pressure. The residue was purified by silica gel flash chromatography (Eluent of 0˜10% EtOAc in petroleum ether) to give tert-butyl 4-(2-cyanopyrimidin-4-yl)piperazine-1-carboxylate (780 mg, yield: 92%) as a white solid. MS: m/z=290.0 [M+H]⁺. ¹H NMR (400 MHz, Dimethylsulfoxide-d₆) δ 8.29 (d, J=6.4 Hz, 1H), 7.10 (d, J=6.4 Hz, 1H), 3.73-3.60 (m, 4H), 3.46-3.41 (m, 4H), 1.42 (s, 9H).

### Step 2: 4-(Piperazin-1-yl)pyrimidine-2-carbonitrile

To a solution of tert-butyl 4-(2-cyanopyrimidin-4-yl)piperazine-1-carboxylate (140 mg, 483 μmol) in CH₂Cl₂ (3 mL) was added TFA (767 mg, 6.73 mmol). The mixture was stirred at 25° C. for 1 hr. The reaction mixture was concentrated under reduced pressure. The crude 4-(piperazin-1-yl)pyrimidine-2-carbonitrile (Intermediate 53, 146 mg TFA salt, yield: 100%) was used in the next step without further purification. MS: m/z=190.0 [M+H]⁺.

### Intermediate 54: 6-(Piperazin-1-yl)pyrimidine-4-carbonitrile

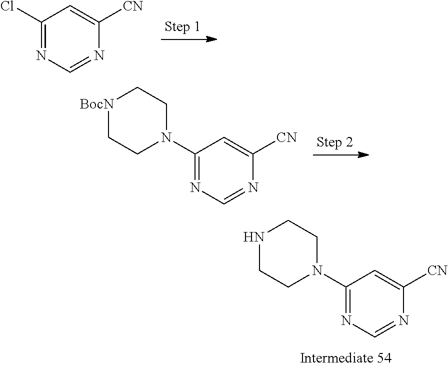 [SMILES: N#Cc1cc(Cl)ncn1 ; CC(C)(C)OC(=O)N1CCN(c2cc(C#N)ncn2)CC1 ; N#Cc1cc(N2CCNCC2)ncn1]

### Step 1: tert-Butyl 4-(6-cyanopyrimidin-4-yl)piperazine-1-carboxylate

To a solution of tert-butyl piperazine-1-carboxylate (700 mg, 3.7 mmol) and 6-chloropyrimidine-4-carbonitrile (524 mg, 3.7 mmol) in DMF (7 mL) were added K₂CO₃ (1.56 g, 11.3 mmol) and NaI (112 mg, 751 μmol). The mixture was stirred at 80° C. for 2 hr. The reaction mixture was diluted with H₂O (20 mL), and then extracted with EtOAc (30 mL×2). The combined organic layers were washed with brine (50 mL), dried over Na₂SO₄, filtered and concentrated under reduced pressure. The residue was purified by silica gel flash chromatography (Eluent of 0˜10^{%} EtOAc in petroleum ether) to give tert-butyl 4-(6-cyanopyrimidin-4-yl)piperazine-1-carboxylate (890 mg, yield: 82%) as a white solid. MS: m/z=290.0 [M+H]⁺. ¹H NMR (400 MHz, Dimethylsulfoxide-d₆) δ 8.58 (s, 1H), 7.56 (s, 1H), 3.75-3.60 (m, 4H), 3.43-3.40 (m, 4H), 1.42 (s, 9H).

### Step 2: 6-(Piperazin-1-yl)pyrimidine-4-carbonitrile

To a solution of tert-butyl 4-(6-cyanopyrimidin-4-yl)piperazine-1-carboxylate (180 mg, 622 μmol) in CH₂Cl₂ (3 mL) was added TFA (1.54 g, 13.5 mmol). The mixture was stirred at 25° C. for 1 hr. The reaction mixture was concentrated under reduced pressure. The crude 6-(piperazin-1-yl)pyrimidine-4-carbonitrile (Intermediate 54, 188 mg, yield: 100%, TFA) was used in the next step without further purification.

### Intermediate 55: 4-(Piperazin-1-yl)-1,3,5-triazine-2-carbonitrile

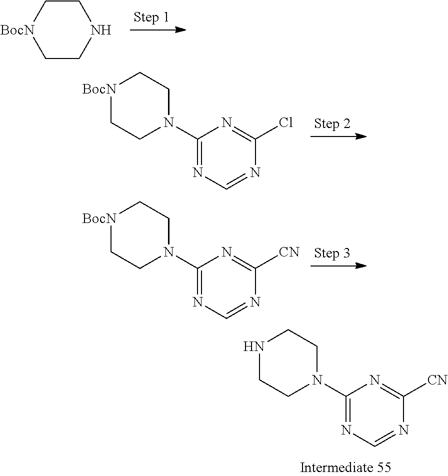

### Step 1: tert-Butyl 4-(4-chloro-1,3,5-triazin-2-yl)piperazine-1-carboxylate

To a solution of tert-butyl piperazine-1-carboxylate (3 g, 1.11 mmol) in THF (70 mL) was added DIEA (3.12 g, 24.2 mmol). The mixture was stirred at 0° C. for 10 min. Then 2,4-dichloro-1,3,5-triazine (2.42 g, 16.1 mmol) in THF (30 mL) was added to the mixture. The mixture was stirred at 25° C. for 16 hr. The mixture was partitioned between EtOAc (600 mL) and H₂O (200 mL). The combined organic layers were washed with H₂O (60 mL) and dried over Na₂SO₄, filtered and concentrated. The residue was purified by silica gel flash chromatography (Eluent of 0˜27% EtOAc in petroleum ether) to give tert-butyl 4-(4-chloro-1,3,5-triazin-2-yl)piperazine-1-carboxylate (3.45 g, yield: 70%) as a light-yellow solid. MS: m/z=300.1 [M+H]+. 1H NMR (400 MHz, Chloroform-d) δ 8.36 (s, 1H), 3.87-3.83 (m, 4H), 3.51-3.48 (m, 4H), 1.48 (s, 9H).

### Step 2: Tert-butyl 4-(4-cyano-1,3,5-triazin-2-yl)piperazine-1-carboxylate

To a solution of tert-butyl 4-(4-chloro-1,3,5-triazin-2-yl)piperazine-1-carboxylate (3.45 g, 11.5 mmol) in DMSO (110 mL) were added KCN (1.64 g, 25.2 mmol) and DABCO (258 mg, 2.30 mmol). The mixture was stirred at 25° C. for 1 hr. The reaction mixture was poured into H₂O (1 L). The resulting mixture was extracted with EtOAc (500 mL×2). The combined organic layers were washed with brine (100 mL), dried over Na₂SO₄, filtered and concentrated under reduced pressure. After purified by silica gel flash chromatography (Eluent of 0˜30% EtOAc in petroleum ether), tert-butyl 4-(4-cyano-1,3,5-triazin-2-yl)piperazine-1-carboxylate (1.5 g, yield: 44%) was obtained as a light-yellow solid. MS: m/z=291.1 [M+H]⁺. ¹H NMR (400 MHz, Chloroform-d) δ 8.57 (s, 1H), 3.89-3.82 (m, 4H), 3.53-3.50 (m, 4H), 1.48 (s, 9H).

### Step 3: 4-(Piperazin-1-yl)-1,3,5-triazine-2-carbonitrile

To a solution of tert-butyl 4-(4-cyano-1,3,5-triazin-2-yl)piperazine-1-carboxylate (200 mg, 689 μmol) in CH₂Cl₂ (3 mL) was added TFA (1.02 g, 8.07 mmol). The mixture was stirred at 25° C. for 1 hr. The reaction mixture was concentrated under reduced pressure. The crude 4-(piperazin-1-yl)-1,3,5-triazine-2-carbonitrile (Intermediate 55, 209 mg TFA salt, yield: 100%) was used in the next step without further purification.

### Intermediate 56: 3-(3-(4-(Chloromethyl)phenyl)-5-morpholino-3H-imidazo[4,5-b]pyridin-2-yl)pyridin-2-amine

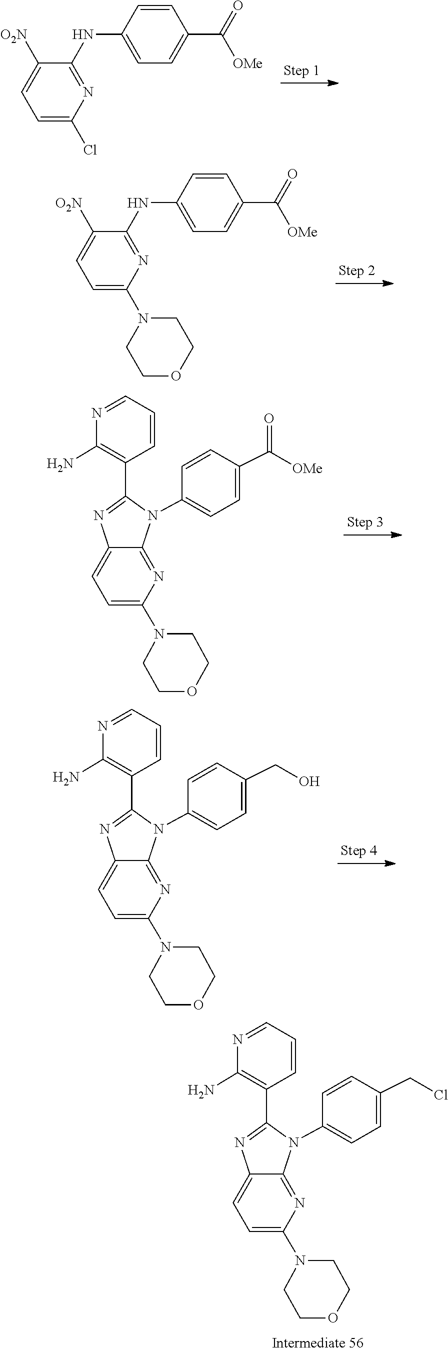

### Step 1: Methyl 4-((6-morpholino-3-nitropyridin-2-yl)amino)benzoate

A mixture of methyl 4-((6-chloro-3-nitropyridin-2-yl)amino)benzoate (refer to Intermediate 13 for detail procedures, 2.5 g, 8.13 mmol), morpholine (849 mg, 9.75 mmol), DIEA (2.10 g, 16.2 mmol) in CH₃CN (30 mL) was stirred at 90° C. for 2 hr. The reaction mixture was filtered and the filter cake was dried under reduced pressure to give methyl 4-((6-morpholino-3-nitropyridin-2-yl)amino)benzoate (2.94 g, yield: 92%) as a yellow solid. MS: m/z=359.0 [M+H]⁺. ¹H NMR (400 MHz, Dimethylsulfoxide-d₆) 10.74 (s, 1H), 8.27 (d, J=9.6 Hz, 1H), 7.98-7.94 (m, 2H), 7.83-7.79 (m, 2H), 6.58 (d, J=9.6 Hz, 1H), 3.84 (s, 3H), 3.75-3.65 (m, 8H).

### Step 2: Methyl 4-(2-(2-aminopyridin-3-yl)-5-morpholino-3H-imidazo[4,5-b]pyridin-3-yl)benzoate

A mixture of methyl 4-((6-morpholino-3-nitropyridin-2-yl)amino)benzoate (2.94 g, 8.20 mmol), 2-aminonicotinaldehyde (1.10 g, 9.02 mmol), and Na₂S₂O₄ (5.71 g, 32.8 mmol) in DMSO (30 mL) was stirred at 100° C. for 16 hr. The reaction mixture was cooled to room temperature and the pH was adjusted to about 9 with sat. NaHCO₃. Then the mixture was diluted with H₂O (100 mL) and extracted with CH₂Cl₂ (100 mL×3). The combined organic layers were washed with brine (100 mL), dried over Na₂SO₄, filtered, and concentrated under reduced pressure. The crude was triturated with EtOAc (100 mL) at 20° C. for 30 min to give methyl 4-(2-(2-aminopyridin-3-yl)-5-morpholino-3H-imidazo[4,5-b]pyridin-3-yl)benzoate (3.5 g, yield: 83%) as a green solid. MS: m/z=431.1 [M+H]⁺. ¹H NMR (400 MHz, Dimethylsulfoxide-d₆) δ 8.07 (d, J=8.4 Hz, 2H), 8.03-7.97 (m, 2H), 7.55 (d, J=8.8 Hz, 2H), 7.32-7.13 (m, 3H), 6.93 (d, J=8.8 Hz, 1H), 6.51-6.44 (m, 1H), 3.88 (s, 3H), 3.70-3.65 (m, 4H), 3.44-3.38 (m, 4H)

### Step 3: (4-(2-(2-Aminopyridin-3-yl)-5-morpholino-3H-imidazo[4,5-b]pyridin-3-yl)phenyl)methanol

A mixture of methyl 4-(2-(2-aminopyridin-3-yl)-5-morpholino-3H-imidazo[4,5-b]pyridin-3-yl)benzoate (3.5 g, 8.13 mmol) in THF (100 mL) was added LiAlH₄ (2.5 M, 3.90 mL) dropwise at 0° C., then the mixture was stirred at 20° C. for 1 hr. The reaction mixture was quenched with Na₂SO₄·10H₂O (5 g) at 0° C., filtered and concentrated under reduced pressure to give (4-(2-(2-aminopyridin-3-yl)-5-morpholino-3H-imidazo[4,5-b]pyridin-3-yl)phenyl)methanol (2.4 g, yield: 63%) as a green solid. MS: m/z=403.1 [M+H]⁺.

### Step 4: 3-(3-(4-(Chloromethyl)phenyl)-5-morpholino-3H-imidazo[4,5-b]pyridin-2-yl)pyridin-2-amine

To a mixture of (4-(2-(2-aminopyridin-3-yl)-5-morpholino-3H-imidazo[4,5-b]pyridin-3yl)phenyl)methanol (2.4 g, 5.96 mmol) in CH₂Cl₂ (16 mL) was added SOCl₂ (4.26 g, 35.7 mmol). The mixture was stirred at 40° C. for 1 hr. The reaction mixture was concentrated under reduced pressure to give 3-(3-(4-(chloromethyl)phenyl)-5-morpholino-3H-imidazo[4,5-b]pyridin-2-yl)pyridin-2-amine (Intermediate 56, 2.6 g, yield: 64%) as a green solid. MS: m/z=421.0 [M+H]⁺.

### Intermediate 57: 3-(2-(2-Aminopyridin-3-yl)-3-(4-(chloromethyl)phenyl)-3H-imidazo[4,5-b]pyridin-5-yl)-1-methylpyridin-2(1H)-one

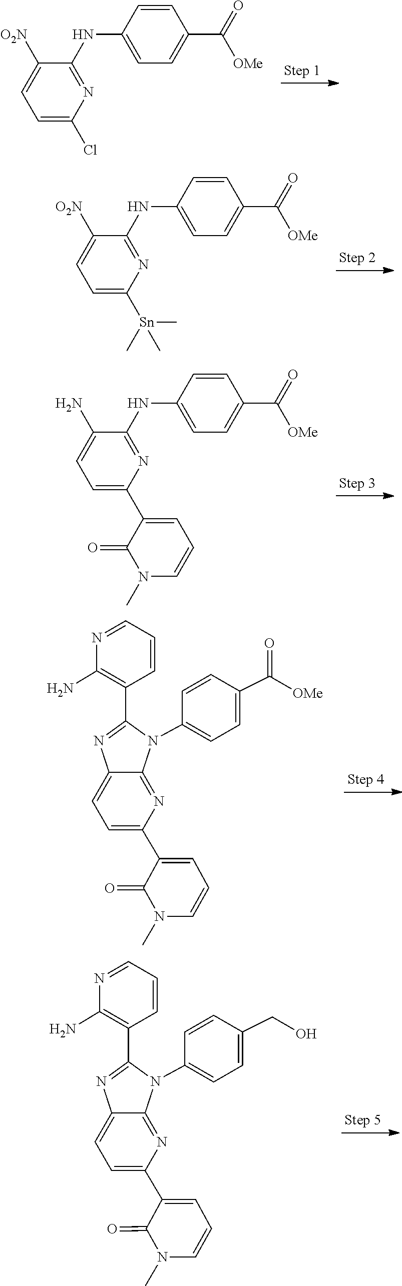

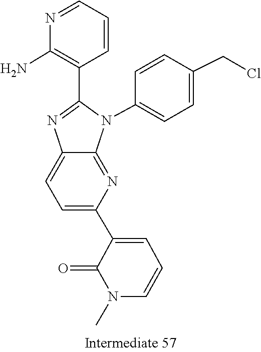 [SMILES: Cn1cccc(-c2ccc3nc(-c4cccnc4N)n(-c4ccc(CCl)cc4)c3n2)c1=O]

### Step 1: Methyl 4-((3-nitro-6-(trimethylstannyl)pyridin-2-yl)amino)benzoate

To a solution of methyl 4-((6-chloro-3-nitropyridin-2-yl)amino)benzoate (refer to Intermediate 13 for detail procedure, 5 g, 16.3 mmol), Pd(PPh₃)₄ (939 mg, 813 μmol) and 1,1,1,2,2,2-hexamethyldistannane (11.1 g, 33.7 mmol) in 1,4-dioxane (100 mL) was degassed and purged with N₂ three times. The mixture was stirred at 90° C. for 16 hr under N₂ atmosphere. The reaction was concentrated under reduced pressure to give methyl 4-((3-nitro-6-(trimethylstannyl)pyridin-2-yl)amino)benzoate (7.09 g, yield: 34%) as a black solid, which was directly used in the next step.

### Step 2: Methyl 4-((1′-methyl-5-nitro-2′-oxo-1′,2′-dihydro-[2,3′-bipyridin]-6-yl)amino)benzoate

A mixture of methyl 4-((3-nitro-6-(trimethylstannyl)pyridin-2-yl)amino)benzoate (7.09 g, 16.3 mmol), 3-bromo-1-methylpyridin-2(1H)-one (3.06 g, 16.3 mmol), and Pd(PPh₃)₄ (939 mg, 813 μmol) in 1,4-dioxane (100 mL) was degassed and purged with N₂ three times, and then the mixture was stirred at 125° C. for 1 hr under N₂ atmosphere. The reaction was concentrated under reduced pressure. After purified by silica gel flash chromatography (Eluent of 1˜5% MeOH in CH₂Cl₂), methyl 4-((1′-methyl-5-nitro-2′-oxo-1′,2′-dihydro-[2,3′-bipyridin]-6-yl)amino)benzoate (3.6 g, purity: 58%) was obtained as a black solid. MS: m/z=351.1 [M+H]⁺.

### Step 3: Methyl 4-(2-(2-aminopyridin-3-yl)-5-(1-methyl-2-oxo-1,2-dihydropyridin-3-yl)-3H-imidazo[4,5-b]pyridin-3-yl)benzoate

To a solution of methyl 4-((5-amino-1′-methyl-2′-oxo-1′,2′-dihydro-[2,3′-bipyridin]-6-yl)amino)benzoate (2.6 g, 7.42 mmol) in DMSO (30 mL) were added Na₂S₂O₄ (5.94 g, 29.7 mmol, 87% purity) and 2-aminonicotinaldehyde (1.09 g, 8.9 mmol). The mixture was stirred at 100° C. for 2 hr. The reaction mixture was quenched with H₂O (50 mL) at 25° C., and then diluted with CH₂Cl₂ (100 mL) and extracted with H₂O (100 mL×3). The combined organic layers were washed with brine (100 mL), dried over Na₂SO₄, filtered, and concentrated under reduced pressure. After purified by silica gel flash chromatography (Eluent of 1˜5% MeOH in CH₂Cl₂), methyl 4-(2-(2-aminopyridin-3-yl)-5-(1-methyl-2-oxo-1,2-dihydropyridin-3-yl)-3H-imidazo[4,5-b]pyridin-3-yl)benzoate (750 mg, yield: 24%) was obtained as a brown solid. MS: m/z=453.1 [M+H]⁺.

### Step 4: 3-(2-(2-Aminopyridin-3-yl)-3-(4-(hydroxymethyl)phenyl)-3H-imidazo[4,5-b]pyridin-5-yl)-1-methylpyridin-2(1H)-one

To a solution of methyl 4-(2-(2-aminopyridin-3-yl)-5-(1-methyl-2-oxo-1,2-dihydropyridin-3-yl)-3H-imidazo[4,5-b]pyridin-3-yl)benzoate (1.0 g, 2.21 mmol) in THF (15 mL) was added LiAlH₄ (1.33 mL, 2.5 M in THF). The mixture was stirred at 0° C. for 1 hr. The reaction mixture was quenched with Na₂SO₄ 10H₂O (2 g) at 0° C., filtered and concentrated under reduced pressure. After purified by silica gel flash chromatography (Eluent of 1˜5% MeOH in CH₂Cl₂), 3-(2-(2-aminopyridin-3-yl)-3-(4-(hydroxymethyl)phenyl)-3H-imidazo[4,5-b]pyridin-5-yl)-1-methylpyridin-2(1H)-one (200 mg, yield: 16%) was obtained as a yellow solid, which was used to the next step directly.

### Step 5: 3-(2-(2-Aminopyridin-3-yl)-3-(4-(chloromethyl)phenyl)-3H-imidazo[4,5-b]pyridin-5-yl)-1-methylpyridin-2(1H)-one

To a solution of 3-(2-(2-aminopyridin-3-yl)-3-(4-(hydroxymethyl)phenyl)-3H-imidazo[4,5-b]pyridin-5-yl)-1-methylpyridin-2(1H)-one (160 mg, 377 μmol) in CH₂Cl₂ (20 mL) was added SOCl₂ (135 mg, 1.13 mmol) at 0° C. The mixture was stirred at 25° C. for 1 hr. The reaction mixture was quenched with H₂O (3 mL) at 0° C., and then filtered and concentrated under reduced pressure to give 3-(2-(2-aminopyridin-3-yl)-3-(4-(chloromethyl)phenyl)-3H-imidazo[4,5-b]pyridin-5-yl)-1-methylpyridin-2(1H)-one (Intermediate 57, 150 mg, yield: 66%) as a yellow solid. MS: m/z=443.0 [M+H]⁺.

### Intermediate 58: 3-(3-(4-(Chloromethyl)phenyl)-5-cyclobutyl-3H-imidazo[4,5-b]pyridin-2-yl)pyridin-2-amine

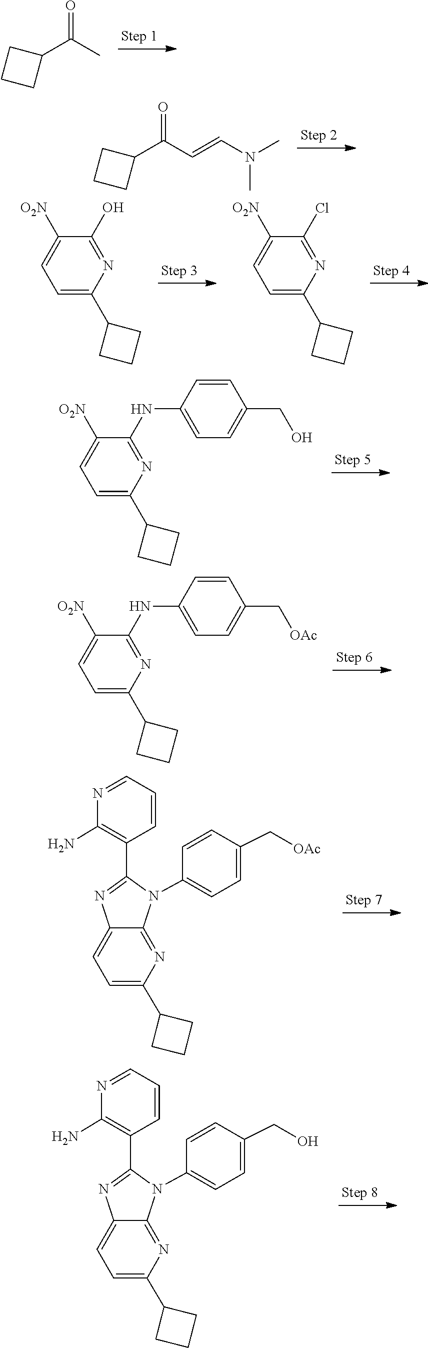

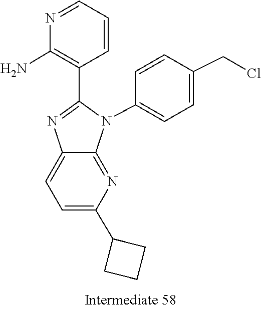 [SMILES: Nc1ncccc1-c1nc2ccc(C3CCC3)nc2n1-c1ccc(CCl)cc1]

### Step 1: (L)-1-Cyclobutyl-3-(dimethylamino)prop-2-en-1-one

To a solution of 1-cyclobutylethan-1-one (8 g, 81.5 mmol) in DMF (20 mL) were added CH₃₀Na (220 mg, 4.08 mmol) and DMF-DMA (19.4 g, 163 mmol). The mixture was stirred at 100° C. for 16 hr. The reaction mixture was quenched with H₂O (50 mL) at 25° C., and then extracted with CH₂Cl₂ (50 mL×3). The combined organic layers were washed with brine (50 mL), dried over Na₂SO₄, filtered and concentrated under reduced pressure to give (E)-1-cyclobutyl-3-(dimethylamino)prop-2-en-1-one (10 g, yield: 74%) as a yellow oil. MS: m/z=154.2 [M+H]⁺.

### Step 2: 6-Cyclobutyl-3-nitropyridin-2-ol

To a solution of (E)-1-cyclobutyl-3-(dimethylamino)prop-2-en-1-one (10 g, 65.3 mmol) in H₂O (100 mL) were added piperidinium acetate (4.74 g, 32.6 mmol) and 2-nitroacetamide (10.2 g, 97.9 mmol). The mixture was stirred at 25° C. for 16 hr. The reaction mixture was extracted with CH₂Cl₂ (150 mL×2). The combined organic layers were washed with brine (150 mL), dried over Na₂SO₄, filtered, and concentrated under reduced pressure. The crude was triturated with EtOAc: petroleum ether=1:10 at 25° C. for 10 min to give 6-cyclobutyl-3-nitropyridin-2-ol (6.5 g, yield: 51%) as a yellow solid. MS: m/z=195.1 [M+H]⁺. ¹H NMR (400 MHz, Dimethylsulfoxide-d₆) δ 12.85-12.40 (m, 1H), 8.42 (d, J=8.0 Hz, 1H), 6.34 (d, J=8.0 Hz, 1H), 3.55-3.44 (m, 1H), 2.30-2.13 (m, 4H), 2.00-1.94 (m, 1H), 1.86-1.77 (m, 1H).

### Step 3: 2-Chloro-6-cyclobutyl-3-nitropyridine

A solution of 6-cyclobutyl-3-nitropyridin-2-ol (6.5 g, 33.5 mmol) in POCl₃ (50 mL) was stirred at 100° C. for 1.5 hr. The reaction mixture was filtered and concentrated under reduced pressure. The crude was quenched with aqueous NaHCO₃ (50 mL) at 10° C., and then extracted with CH₂Cl₂ (50 mL×3). The combined organic layers were washed with brine (100 mL), dried over Na₂SO₄, filtered, and concentrated under reduced pressure to give 2-chloro-6-cyclobutyl-3-nitropyridine (5.5 g, yield: 76%) as a black oil. MS: m/z=213.1 [M+H]⁺. ¹H NMR (400 MHz, Dimethylsulfoxide-d₆) δ 8.48 (d, J=8.0 Hz, 1H), 7.56 (d, J=8.4 Hz, 1H), 3.83-3.71 (m, 1H), 2.34-2.22 (m, 4H), 2.07-1.96 (m, 1H), 1.92-1.79 (m, 1H).

### Step 4: (4-((6-Cyclobutyl-3-nitropyridin-2-yl)amino)phenyl)methanol

To a solution of 2-chloro-6-cyclobutyl-3-nitropyridine (5.5 g, 25.9 mmol) in DMSO (60 mL) were added DIEA (10 g, 77.6 mmol) and (4-aminophenyl)methanol (3.19 g, 25.9 mmol). The mixture was stirred at 100° C. for 16 hr. The reaction mixture was quenched with H₂O (200 mL) at 25° C., and extracted with CH₂Cl₂ (500 mL×3). The combined organic layers were washed with brine (500 mL), dried over Na₂SO₄, filtered and concentrated under reduced pressure to give (4-((6-cyclobutyl-3-nitropyridin-2-yl)amino)phenyl)methanol (7.74 g, yield: 83%) as a brown solid. MS: m/z=300.1 [M+H]⁺.

### Step 5: 4-((6-Cyclobutyl-3-nitropyridin-2-yl)amino)benzyl acetate

To a solution of (4-((6-cyclobutyl-3-nitropyridin-2-yl)amino)phenyl)methanol (7.7 g, 25.7 mmol) in CH₂Cl₂ (100 mL) were added TEA (7.81 g, 77.2 mmol), Ac₂O (3.94 g, 38.6 mmol) and DMAP (314 mg, 2.57 mmol). The mixture was stirred at 25° C. for 1 hr. The reaction mixture was quenched with H₂O (100 mL) at 25° C., and then extracted with CH₂Cl₂ (100 mL×3). The combined organic layers were washed with brine (100 mL), dried over Na₂SO₄, filtered and concentrated under reduced pressure. After purified by silica gel flash chromatography (Eluent of 1˜5% EtOAc in petroleum ether), 4-((6-cyclobutyl-3-nitropyridin-2-yl)amino)benzyl acetate (5.65 g, yield: 64%) was obtained as a yellow oil. MS: m/z=342.0 [M+H]⁺.

### Step 6: 4-(2-(2-Aminopyridin-3-yl)-5-cyclobutyl-3H-imidazo[4,5-b]pyridin-3-yl)benzyl acetate acetate

To a solution of 4-((6-cyclobutyl-3-nitropyridin-2-yl)amino)benzyl acetate (5.6 g, 16.4 mmol) in DMSO (60 mL) were added Na₂S₂O₄ (13.1 g, 65.6 mmol, 87% purity) and 2-aminonicotinaldehyde (2.4 g, 19.7 mmol). The mixture was stirred at 100° C. for 16 hr. The reaction mixture was quenched with H₂O (100 mL) at 25° C., and then extracted with CH₂Cl₂ (150 mL×2). The combined organic layers were washed with brine (150 mL), dried over Na₂SO₄, filtered and concentrated under reduced pressure. After purified by silica gel flash chromatography (Eluent of 1˜50% CH₂Cl₂ in EtOAc), 4-(2-(2-aminopyridin-3-yl)-5-cyclobutyl-3H-imidazo[4,5-b]pyridin-3-yl)benzyl acetate acetate (2.1 g, yield: 31%) was obtained as a yellow solid. MS: m/z=414.1 [M+H]⁺. ¹H NMR (400 MHz, Dimethylsulfoxide-d₆) δ 8.10 (d, J=8.4 Hz, 1H), 7.98 (d, J=4.4 Hz, 1H), 7.52 (d, J=8.0 Hz, 2H), 7.43 (d, J=8.0 Hz, 2H), 7.30 (d, J=8.4 Hz, 1H), 7.16 (d, J=7.2 Hz, 1H), 6.85 (br s, 2H), 6.41 (dd, J=7.6, 5.2 Hz, 11H), 5.16 (s, 2H), 3.74-3.64 (m, 1H), 2.27-2.20 (m, 4H), 2.11 (s, 3H), 1.99-1.92 (m, 1H), 1.84-1.75 (m, 1H).

### Step 7: (4-(2-(2-Aminopyridin-3-yl)-5-cyclobutyl-3H-imidazo[4,5-b]pyridin-3-yl)phenyl)methanol

To a solution of 4-(2-(2-aminopyridin-3-yl)-5-cyclobutyl-3H-imidazo[4,5-b]pyridin-3-yl)benzyl acetate acetate (500 mg, 1.21 mmol) in MeOH (5 mL), THF (5 mL) and H₂O (2 mL) was added K₂CO₃ (501 mg, 3.63 mmol). The mixture was stirred at 25° C. for 0.5 hr. The reaction mixture was diluted with H₂O (10 mL) and extracted with CH₂Cl₂ (15 mL×2). The combined organic layers were washed with brine (15 mL), dried over Na₂SO₄, filtered and concentrated under reduced pressure to give (4-(2-(2-aminopyridin-3-yl)-5-cyclobutyl-3H-imidazo[4,5-b]pyridin-3-yl)phenyl)methanol (450 mg, yield: 86%) as a yellow solid. MS: m/z=372.0 [M+H]⁺.

### Step 8: 3-(3-(4-(Chloromethyl)phenyl)-5-cyclobutyl-3H-imidazo[4,5-b]pyridin-2-yl)pyridin-2-amine

To a solution of (4-(2-(2-aminopyridin-3-yl)-5-cyclobutyl-3H-imidazo[4,5-b]pyridin-3-yl)phenyl)methanol (449 mg, 1.21 mmol) in CH₂Cl₂ (10 mL) was added SOCl₂ (288 mg, 2.42 mmol) at 0° C. The mixture was stirred at 25° C. for 0.3 hr. The reaction was concentrated under reduced pressure to give 3-(3-(4-(chloromethyl)phenyl)-5-cyclobutyl-3H-imidazo[4,5-b]pyridin-2-yl)pyridin-2-amine (Intermediate 58, 560 mg, HCl salt, yield: 90°/%) as a yellow solid. MS: m/z=390.1[M+H]⁺.

### Intermediate 59: 3-(3-(4-((4-Aminopiperidin-1-yl)methyl)phenyl)-5-(cyclohex-1-en-1-yl)-3H-imidazo[4,5-b]pyridin-2-yl)pyridin-2-amine

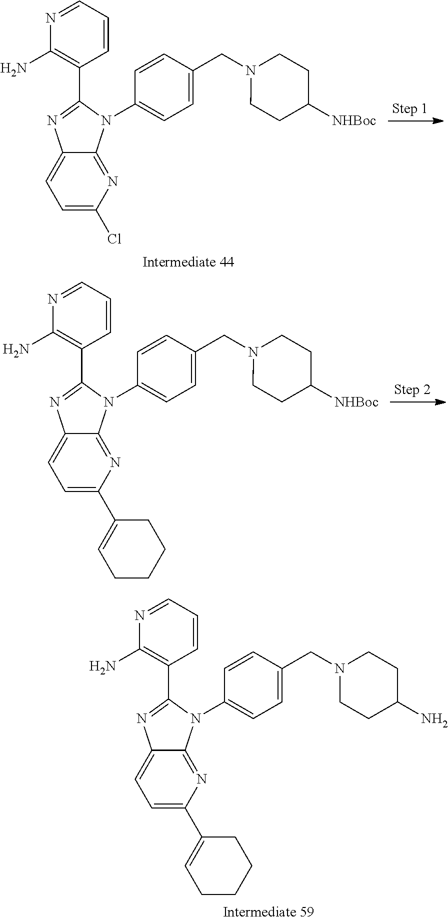

### Step 1: Tert-butyl (1-(4-(2-(2-aminopyridin-3-yl)-5-(cyclohex-1-en-1-yl)-3H-imidazo[4,5-b]pyridin-3-yl)benzyl)piperidin-4-yl)carbamate

A mixture of Intermediate 44 (800 mg, 1.5 mmol), cyclohex-1-en-1-ylboronic acid (283 mg, 2.25 mmol), Cs₂CO₃ (974 mg, 3.00 mmol), and Pd(dppf)Cl₂ (219 mg, 299 μmol) in 1,4-dioxane (2 mL) and H₂O (0.5 mL) was degassed and purged with N₂ three times, and then the mixture was stirred at 100° C. for 16 hr under N₂ atmosphere. The reaction mixture was poured into H₂O (10 mL), extracted with EtOAc (15 mL×3). The combined organic layers were dried over anhydrous Na₂SO₄, filtered and concentrated under reduced pressure. The residue was purified by silica gel flash chromatography (Eluent of 0˜7% MeOH in CH₂Cl₂) to give tert-butyl (1-(4-(2-(2-aminopyridin-3-yl)-5-(cyclohex-1-en-1-yl)-3H-imidazo[4,5-b]pyridin-3-yl)benzyl)piperidin-4-yl)carbamate (702 mg, yield: 81%) as a black brown solid. MS: m/z=580.2 [M+H]⁺.

### Step 2: 3-(3-(4-((4-Aminopiperidin-1-yl)methyl)phenyl)-5-(cyclohex-1-en-1-yl)-3H-imidazo[4,5-b]pyridin-2-yl)pyridin-2-amine

To a solution of tert-butyl (1-(4-(2-(2-aminopyridin-3-yl)-5-(cyclohex-1-en-1-yl)-3H-imidazo[4,5-b]pyridin-3-yl)benzyl)piperidin-4-yl)carbamate (200 mg, 345 μmol) in CH₂Cl₂ (2 mL) was added 4 M HCl in 1,4-dioxane (2.0 mL). After concentrated under reduced pressure, 3-(3-(4-((4-Aminopiperidin-1-yl)methyl)phenyl)-5-(cyclohex-1-en-1-yl)-3H-imidazo[4,5-b]pyridin-2-yl)pyridin-2-amine (Intermediate 59, 160 mg HCl salt, yield: 90%) was obtained as an off-white solid. MS: m/z=480.3 [M+H]⁺. ¹H NMR (400 MHz, Dimethylsulfoxide-d₆) δ 8.10 (d, J=8.4 Hz, 1H), 7.98 (dd, J=4.8, 1.2 Hz, 1H), 7.53 (d, J=8.0 Hz, 1H), 7.45-7.40 (m, 2H), 7.38-7.32 (m, 2H), 7.14-7.07 (m, 1H), 7.01 (br s, 2H), 6.67-6.60 (m, 1H), 6.34 (dd, J=7.6, 4.8 Hz, 1H), 3.52 (br s, 2H), 3.49-3.48 (m, 1H), 2.76-2.73 (m, 2H), 2.44-2.43 (m, 2H), 2.20-2.19 (m, 2H), 2.02-1.96 (m, 2H), 1.72-1.65 (m, 4H), 1.61-1.54 (m, 2H), 1.27-1.23 (m, 2H).

### Intermediate 60: 3-(3-(4-((4-Aminopiperidin-1-yl)methyl)phenyl)-5-cyclohexyl-3H-imidazo[4,5-b]pyridin-2-yl)pyridin-2-amine

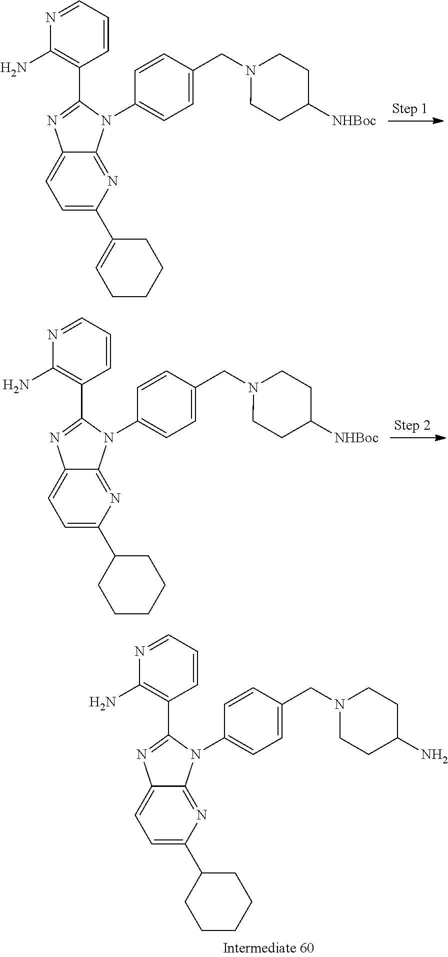

### Step 1: tert-Butyl (1-(4-(2-(2-aminopyridin-3-yl)-5-cyclohexyl-3H-imidazo[4,5-b]pyridin-3-yl)benzyl)piperidin-4-yl)carbamate

To a solution of tert-butyl (1-(4-(2-(2-aminopyridin-3-yl)-5-(cyclohex-1-en-1-yl)-3H-imidazo[4,5-b]pyridin-3-yl)benzyl)piperidin-4-yl)carbamate (refer to Intermediate 59 for detail procedures, 242 mg, 417 μmol) in MeOH (10 mL) was added Pd/C (50 mg, 834 μmol, 10% w/w). The suspension was degassed under reduced pressure and purged with H₂ several times. The mixture was stirred under H₂ (50 psi) at 50° C. for 24 hr under H₂. The reaction mixture was cooled to 25° C., then filtered and concentrated under reduced pressure. The residue was purified by silica gel flash chromatography (Eluent of 0˜82% EtOAc in petroleum ether) to give tert-butyl (1-(4-(2-(2-aminopyridin-3-yl)-5-cyclohexyl-3H-imidazo[4,5-b]pyridin-3-yl)benzyl)piperidin-4-yl)carbamate (182 mg, yield: 75%) as a yellow solid. MS: m/z=582.3 [M+H]⁺.

### Step 2: 3-(3-(4-((4-Aminopiperidin-1-yl)methyl)phenyl)-5-cyclohexyl-3H-imidazo[4,5-b]pyridin-2-yl)pyridin-2-amine

To a solution of tert-butyl (1-(4-(2-(2-aminopyridin-3-yl)-5-cyclohexyl-3H-imidazo[4,5-b]pyridin-3-yl)benzyl)piperidin-4-yl)carbamate (182 mg, 345 μmol) in 1,4-dioxane (2 mL) was added 4 M HCl in 1,4-dioxane (2.0 mL). The mixture was stirred at 25° C. for 1 hr. The reaction mixture was filtered to give 3-(3-(4-((4-Aminopiperidin-1-yl)methyl)phenyl)-5-cyclohexyl-3H-imidazo[4,5-b]pyridin-2-yl)pyridin-2-amine (Intermediate 60, 162 mg HCl salt, yield: 91%) was obtained as a yellow solid. MS: m/z=482.1 [M+H]⁺. ¹H NMR (400 MHz, Methanol-d) δ 8.25-8.11 (m, 1H), 7.99-7.92 (m, 1H), 7.93-7.77 (m, 3H), 7.65 (d, J=8.0 Hz, 2H), 7.50-7.35 (m, 1H), 6.95-6.73 (m, 1H), 3.70 (d, J=12.0 Hz, 2H), 3.64-3.40 (m, 2H), 3.33-3.31 (m, 3H), 2.88-2.70 (m, 1H), 2.30 (d, J=12.8 Hz, 2H), 2.21-2.02 (m, 2H), 1.95-1.81 (m, 4H), 1.79-1.71 (m, 1H), 1.63-1.49 (m, 2H), 1.39 (m, 2H), 1.35-1.24 (m, 1H).

### Intermediate 61: 3-(3-(4-((4-Aminopiperidin-1-yl)methyl)phenyl)-2-(2-aminopyridin-3-yl)-3H-imidazo[4,5-b]pyridin-5-yl)benzonitrile

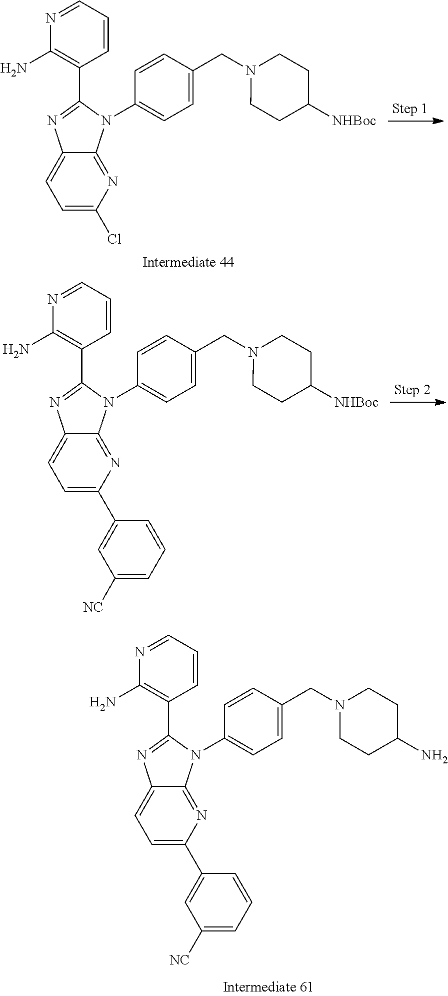 [SMILES: CC(C)(C)OC(=O)NC1CCN(Cc2ccc(-n3c(-c4cccnc4N)nc4ccc(Cl)nc43)cc2)CC1 ; CC(C)(C)OC(=O)NC1CCN(Cc2ccc(-n3c(-c4cccnc4N)nc4ccc(-c5cccc(C#N)c5)nc43)cc2)CC1 ; N#Cc1cccc(-c2ccc3nc(-c4cccnc4N)n(-c4ccc(CN5CCC(N)CC5)cc4)c3n2)c1]

### Step 1: tert-Butyl (1-(4-(2-(2-aminopyridin-3-yl)-5-(3-cyanophenyl)-3H-imidazo[4,5-b]pyridin-3-yl)benzyl)piperidin-4-yl)carbamate

A mixture of Intermediate 44 (350 mg, 655 μmol), (3-cyanophenyl)boronic acid (116 mg, 786 μmol), K₂CO₃ (181 mg, 1.31 mmol), and Pd(dppf)Cl₂ (24 mg, 33 μmol) in 1,4-dioxane (10 mL) and H₂O (2 mL) was degassed and purged with N₂ three times, and then the mixture was stirred at 100° C. for 16 hr under N₂ atmosphere. The reaction mixture was poured into H₂O (3 mL) and extracted with EtOAc (15 mL). The combined organic layers were dried over anhydrous Na₂SO₄, filtered and concentrated under reduced pressure. The residue was purified by silica gel flash chromatography (Eluent of 0˜9% MeOH in CH₂Cl₂) to give tert-butyl (1-(4-(2-(2-aminopyridin-3-yl)-5-(3-cyanophenyl)-3H-imidazo[4,5-b]pyridin-3-yl)benzyl)piperidin-4-yl)carbamate (150 mg, yield: 38%) as a yellow solid. MS: m/z=601.4 [M+H]⁺.

### Step 2: 3-(3-(4-((4-Aminopiperidin-1-yl)methyl)phenyl)-2-(2-aminopyridin-3-yl)-3H-imidazo[4,5-b]pyridin-5-yl)benzonitrile

To a solution of tert-butyl (1-(4-(2-(2-aminopyridin-3-yl)-5-(3-cyanophenyl)-3H-imidazo[4,5-b]pyridin-3-yl)benzyl)piperidin-4-yl)carbamate (50 mg, 73 μmol) in 1,4-dioxane (2 mL) was added 4 M HCl in 1,4-dioxane (2 mL). The mixture was stirred at 25° C. for 1 hr. The reaction mixture was filtered to give the filter cake. 3-(3-(4-((4-Aminopiperidin-1-yl)methyl)phenyl)-2-(2-aminopyridin-3-yl)-3H-imidazo[4,5-b]pyridin-5-yl)benzonitrile (Intermediate 61, 32 mg HCl salt, yield: 87%) was obtained as a yellow solid. MS: m/z=501.3 [M+H]⁺. ¹H NMR (400 MHz, Methanol-d₄) δ 8.48-8.32 (m, 3H), 8.14 (d, J=8.8 Hz, 1H), 8.06 (d, J=5.6 Hz, 1H), 7.99-7.87 (m, 3H), 7.83-7.73 (m, 3H), 7.71-7.64 (m, 1H), 6.93-6.89 (m, 1H), 3.79-3.68 (m, 2H), 3.67-3.45 (m, 3H), 3.37 (s, 2H), 2.35-2.33 (m, 2H), 2.23-2.05 (m, 2H).

### Intermediate 62: 5-(3-(4-((4-aminopiperidin-1-yl)methyl)phenyl)-2-(2-aminopyridin-3-yl)-3H-imiidazo[4,5-b]pyridin-5-yl)pyridin-2(11H)-one

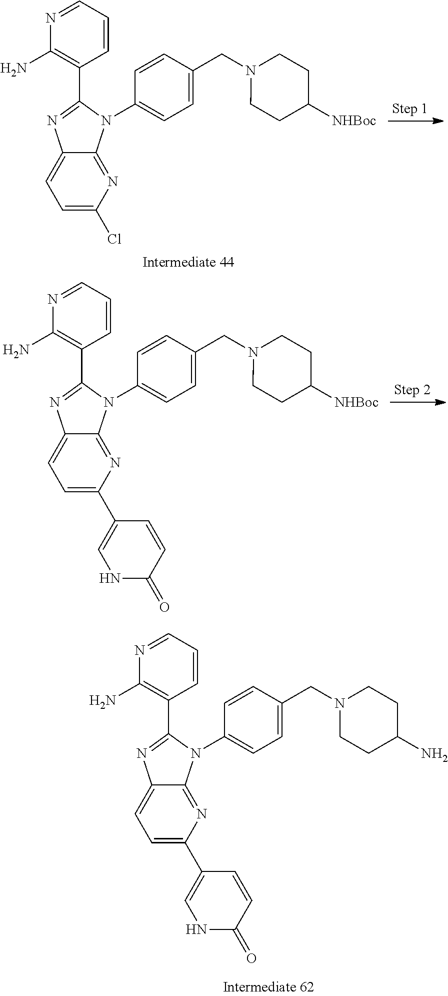

### Step 1: tert-Butyl (1-(4-(2-(2-aminopyridin-3-yl)-5-(6-oxo-1,6-dihydropyridin-3-yl)-3H-imidazo[4,5-b]pyridin-3-yl)benzyl)piperidin-4-yl)carbamate

A mixture of Intermediate 44 (100 mg, 187 μmol), (6-oxo-1,6-dihydropyridin-3-yl)boronic acid (39 mg, 281 μmol), Cs₂CO₃ (122 mg, 375 μmol), Pd(dppf)Cl₂ (14 mg, 19 μmol) in H₂O (2 mL) and 1,4-dioxane (8 mL) was degassed and purged with N₂ three times, and then the mixture was stirred at 100° C. for 1 hr under N₂ atmosphere. The reaction mixture was quenched with H₂O (10 mL) at 25° C. and extracted with EtOAc (15 mL×2). The combined organic layers were washed with brine (20 mL), dried over Na₂SO₄, filtered, and concentrated under reduced pressure. The crude was purified by silica gel flash chromatography (Eluent of 0˜10% MeOH in CH₂Cl₂) to give tert-butyl (1-(4-(2-(2-aminopyridin-3-yl)-5-(6-oxo-1,6-dihydropyridin-3-yl)-3H-imidazo[4,5-b]pyridin-3-yl)benzyl)piperidin-4-yl)carbamate (52 mg, yield: 47%) as a light-yellow solid. MS: m/z=593.3 [M+H]+. ¹H NMR (400 MHz, Chloroform-d) δ 12.64-12.02 (m, 1H), 8.11-8.03 (m, 4H), 7.54 (d, J=8.4 Hz, 1H), 7.48 (d, J=8.0 Hz, 2H), 7.33 (d, J=7.6 Hz, 2H), 7.06 (d, J=7.6 Hz, 1H), 6.70-6.58 (m, 3H), 6.34 (dd, J=7.6, 4.8 Hz, 1H), 4.53-4.38 (m, 1H), 3.61 (s, 2H), 3.50-3.48 (m, 1H), 2.92-2.80 (m, 2H), 2.22-2.13 (m, 2H), 2.00-1.93 (m, 2H), 1.52-1.48 (m, 2H), 1.45 (s, 9H).

### Step 2: 5-(3-(4-((4-Aminopiperidin-1-yl)methyl)phenyl)-2-(2-aminopyridin-3-yl)-3H-imidazo[4,5-b]pyridin-5-yl)pyridin-2(1H)-one

To a solution of tert-butyl (1-(4-(2-(2-aminopyridin-3-yl)-5-(6-oxo-1,6-dihydropyridin-3-yl)-3H-imidazo[4,5-b]pyridin-3-yl)benzyl)piperidin-4-yl)carbamate (50 mg, 84 μmol) in HCl in 1,4-dioxane (4 M, 2 mL). The mixture was stirred at 25° C. for 1 hr. Then the mixture was concentrated under reduced pressure to give 5-(3-(4-((4-aminopiperidin-1-yl)methyl)phenyl)-2-(2-aminopyridin-3-yl)-3H-imidazo[4,5-b]pyridin-5-yl)pyridin-2(1H)-one (Intermediate 62, 33.3 mg, yield: 72%) as a light-yellow solid. MS: m/z=493.2 [M+H]⁺. ¹H NMR (400 MHz, Methanol-d₄) δ 8.59-8.52 (m, 1H), 8.45-8.39 (m, 1H), 8.33 (d, J=8.4 Hz, 1H), 8.05-8.01 (m, 1H), 7.97 (d, J=8.4 Hz, 1H), 7.92-7.86 (m, 3H), 7.73-7.69 (m, 2H), 6.92-6.84 (m, 2H), 3.73-3.67 (m, 2H), 3.67-3.65 (m, 1H), 3.62-3.57 (m, 2H), 3.38-3.34 (m, 2H), 2.35-2.30 (m, 2H), 2.21-2.13 (m, 2H).

### Intermediate 63: 6-(3-(4-((4-aminopiperidin-1-yl)methyl)phenyl)-2-(2-aminopyridin-3-yl)-3H-imidazo[4,5-b]pyridin-5-yl)pyridin-2(1H)-one

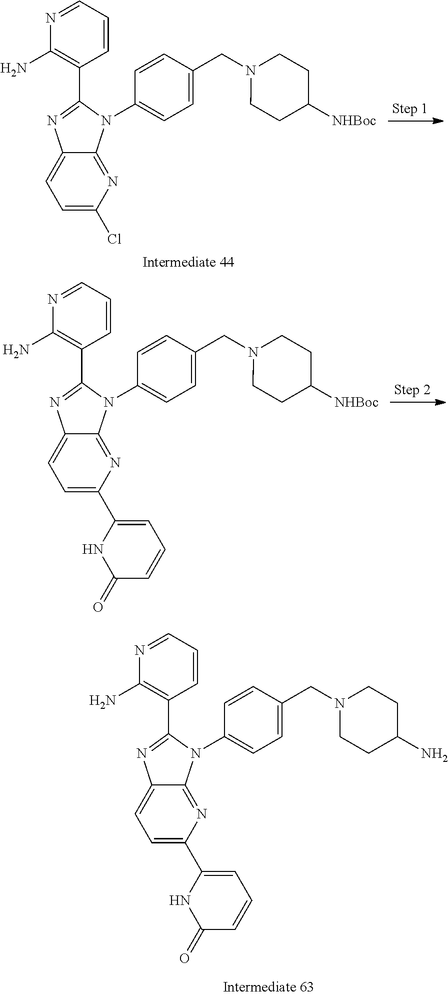 [SMILES: CC(C)(C)OC(=O)NC1CCN(Cc2ccc(-n3c(-c4cccnc4N)nc4ccc(Cl)nc43)cc2)CC1 ; CC(C)(C)OC(=O)NC1CCN(Cc2ccc(-n3c(-c4cccnc4N)nc4ccc(-c5cccc(=O)[nH]5)nc43)cc2)CC1 ; Nc1ncccc1-c1nc2ccc(-c3cccc(=O)[nH]3)nc2n1-c1ccc(CN2CCC(N)CC2)cc1]

### Step 1: tert-Butyl (1-(4-(2-(2-aminopyridin-3-yl)-5-(6-oxo-1,6-dihydropyridin-2-yl)-3H-imidazo[4,5-b]pyridin-3-yl)benzyl)piperidin-4-yl)carbamate

A mixture of Intermediate 44 (100 mg, 187 μmol), 6-(4,4,5,5-tetramethyl-1,3,2-dioxaborolan-2-yl)pyridin-2(1H)-one (62 mg, 281 μmol), Cs₂CO₃ (122 mg, 374 μmol), Pd(dppf)Cl₂ (14 mg, 19 μmol) in H₂O (2 mL) and 1,4-dioxane (8 mL) was degassed and purged with N₂ three times, and then the mixture was stirred at 100° C. for 1 hr under N₂ atmosphere. The reaction mixture was quenched with H₂O (10 mL) at 25° C. and extracted with EtOAc (15 mL×2). The combined organic layers were washed with brine (20 mL), dried over Na₂SO₄, filtered and concentrated under reduced pressure. The crude was purified by silica gel flash chromatography (Eluent of 0˜10% MeOH in CH₂Cl₂) to give tert-butyl (1-(4-(2-(2-aminopyridin-3-yl)-5-(6-oxo-1,6-dihydropyridin-2-yl)-3H-imidazo[4,5-b]pyridin-3-yl)benzyl)piperidin-4-yl)carbamate (100 mg, yield: 90%) as a light-yellow solid. MS: m/z=593.3 [M+H]⁺. ¹H NMR (400 MHz, Chloroform-d) δ 10.25 (br s, 1H), 8.17 (d, J=8.4 Hz, 1H), 8.08 (dd, J=4.8, 2.0 Hz, 1H), 7.86 (d, J=8.8 Hz, 1H), 7.58-7.53 (m, 2H), 7.50-7.46 (m, 1H), 7.31 (d, J=8.0 Hz, 2H), 7.05 (dd, J=7.6, 1.6 Hz, 1H), 6.87 (d, 1=6.8 Hz, 1H), 6.65 (br s, 2H), 6.58 (d, J=8.0 Hz, 1H), 6.33 (dd, J=7.6, 4.8 Hz, 1H), 4.54-4.42 (m, 1H), 3.66 (s, 2H), 3.59-3.50 (m, 1H), 2.97-2.86 (m, 2H), 2.30-2.18 (m, 2H), 2.01-1.95 (m, 2H), 1.56-1.50 (m, 2H), 1.45 (s, 9H)

### Step 2: 6-(3-(4-((4-Aminopiperidin-1-yl)methyl)phenyl)-2-(2-aminopyridin-3-yl)-3H-imidazo[4,5-b]pyridin-5-yl)pyridin-2(1H)-one

To a solution of tert-butyl (1-(4-(2-(2-aminopyridin-3-yl)-5-(6-oxo-1,6-dihydropyridin-2-yl)-3H-imidazo[4,5-b]pyridin-3-yl)benzyl)piperidin-4-yl)carbamate (100.00 mg, 169 μmol) in HCl in 1,4-dioxane (4 M, 3 mL). The mixture was stirred at 25° C. for 1 hr. Then the mixture was concentrated under reduced pressure to give 6-(3-(4-((4-aminopiperidin-1-yl)methyl)phenyl)-2-(2-aminopyridin-3-yl)-3H-imidazo[4,5-b]pyridin-5-yl)pyridin-2(1H)-one (Intermediate 63, 62.6 mg, yield: 67%) as a light-yellow solid. MS: m/z=493.2 [M+H]⁺. ¹H NMR (400 MHz, Methanol-d₄) δ 8.47 (d, J=8.4 Hz, 1H), 8.25 (d, J=8.0 Hz, 1H), 8.08-7.98 (m, 2H), 7.96-7.89 (m, 3H), 7.78-7.72 (m, 2H), 7.50-7.58 (m, 1H), 6.94-6.82 (m, 2H), 3.76-3.68 (m, 2H), 3.66 (s, 2H), 3.61-3.56 (m, 1H), 3.38-3.32 (m, 2H), 2.36-2.29 (m, 2H), 2.21-2.08 (m, 2H).

### Intermediate 64: 4-(2,7-Diazaspiro[3.5]nonan-7-yl)-1,3,5-triazine-2-carbonitrile

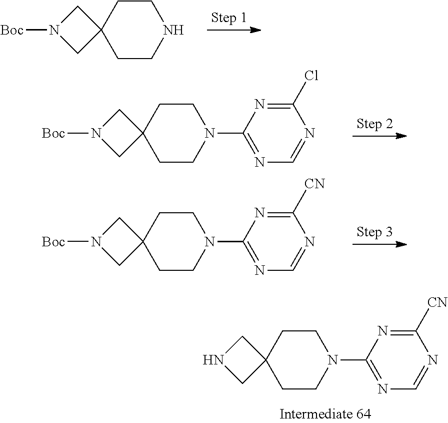

### Step 1: tert-Butyl 7-(4-chloro-1,3,5-triazin-2-yl)-2,7-diazaspiro[3.5]nonane-2-carboxylate

To a solution of tert-butyl 2,7-diazaspiro[3.5]nonane-2-carboxylate (400 mg, 1.77 mmol) and 2,4-dichloro-1,3,5-triazine (265 mg, 1.77 mmol) in THF (10 mL) was added DIEA (685 mg, 5.3 mmol). The resulting mixture was stirred at 0° C. for 1 hr. Then the reaction mixture was diluted with H₂O (50 mL) and extracted with CH₂Cl₂ (50 mL×2). The combined organic layers were dried over Na₂SO₄, filtered and concentrated under reduced pressure. tert-Butyl 7-(4-chloro-1,3,5-triazin-2-yl)-2,7-diazaspiro[3.5]nonane-2-carboxylate (500 mg, yield: 78%) was obtained as a white solid. MS: m/z=340.0 [M+H]⁺. ¹H NMR (400 MHz, Chloroform-d) δ 8.34 (s, 1H), 3.84-3.78 (m, 4H), 3.71 (s, 4H), 1.83-1.79 (m, 4H), 1.46 (s, 9H).

### Step 2: tert-Butyl 7-(4-cyano-1,3,5-triazin-2-yl)-2,7-diazaspiro[3.5]nonane-2-carboxylate

To a solution of tert-butyl 7-(4-chloro-1,3,5-triazin-2-yl)-2,7-diazaspiro[3.5]nonane-2-carboxylate (500 mg, 1.47 mmol) in DMSO (10 mL) were added KCN (660 mg, 10.1 mmol) and DABCO (33 mg, 294 μmol). The mixture was stirred at 25° C. for 16 hr. The reaction mixture was poured into H₂O (50 mL). The resulting mixture was extracted with EtOAc (50 mL×2). The combined organic layers were washed with brine (50 mL), dried over Na₂SO₄, filtered and concentrated under reduced pressure. After purified by silica gel flash chromatography (Eluent of 20˜30% EtOAc in petroleum ether), tert-butyl 7-(4-cyano-1,3,5-triazin-2-yl)-2,7-diazaspiro[3.5]nonane-2-carboxylate (200 mg, yield: 37%) was obtained as a white solid. MS: m/z=275.1 [M+H−56]⁺.

### Step 3: 4-(2,7-Diazaspiro[3.5]nonan-7-yl)-1,3,5-triazine-2-carbonitrile

To a solution of tert-butyl 7-(4-cyano-1,3,5-triazin-2-yl)-2,7-diazaspiro[3.5]nonane-2-carboxylate (200 mg, 605 μmol) in CH₂Cl₂ (5 mL) was added TFA (207 mg, 1.82 mmol). The mixture was stirred at 25° C. for 0.5 hr. The reaction mixture was filtered and concentrated under reduced pressure to give 4-(2,7-diazaspiro[3.5]nonan-7-yl)-1,3,5-triazine-2-carbonitrile (Intermediate 64, 130 mg TFA salt, yield: 56%) as a yellow oil. MS: m/z=231.1 [M+H]⁺. ¹H NMR (400 MHz, Dimethylsulfoxide-d₆) δ 8.74-8.68 (m, 3H), 3.82-3.76 (m, 6H), 3.75-3.71 (m, 2H), 1.86-1.80 (m, 4H).

### Intermediate 65: 4-(7-Azaspiro[3.5]nonan-2-ylamino)-1,3,5-triazine-2-carbonitrile

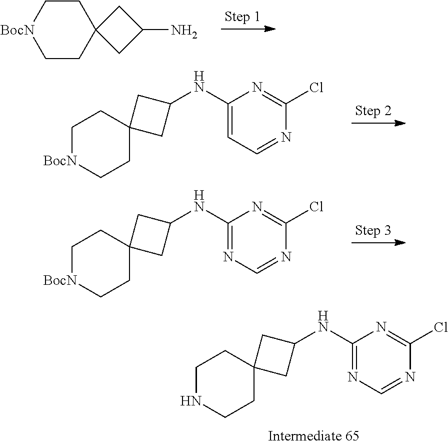

### Step 1: tert-Butyl 2-((2-chloropyrimidin-4-yl)amino)-7-azaspiro[3.5]nonane-7-carboxylate

To a solution of tert-butyl 2-amino-7-azaspiro[3.5]nonane-7-carboxylate (2.0 g, 8.32 mmol) in THF (50 mL) was added DIPEA (1.61 g, 12.5 mmol). The mixture was stirred at 0° C. for 10 min, and then 2,4-dichloro-1,3,5-triazine (1.258 g, 8.32 mmol) in THF (20 mL) was added dropwise at 0° C. The resulting mixture was stirred at 0° C. for 4 hr. The reaction mixture diluted with H₂O (100 mL) and extracted with EtOAc (50 mL×2). The combined organic layers were washed with brine (100 mL), dried over Na₂SO₄, filtered and concentrated under reduced pressure. The residue was purified by silica gel flash chromatography (Eluent of 0˜30% EtOAc in petroleum ether) to give tert-butyl 2-[(4-chloro-1,3,5-triazin-2-yl)amino]-7-azaspiro[3.5]nonane-7-carboxylate (1.6 g, yield: 54% yield) was obtained as a white solid. MS: m/z=354.1 [M+H]+. ¹H NMR (400 MHz, Chloroform-d) 8.42-8.30 (m, 1H), 5.81-5.69 (m, 1H), 4.53-4.42 (m, 1H), 3.39-3.36 (m, 2H), 3.32-3.28 (m, 2H), 2.45-2.39 (m, 2H), 1.73-1.67 (m, 2H), 1.64-1.60 (m, 2H), 1.55-1.51 (m, 2H), 1.45 (s, 9H).

### Step 2: tert-Butyl 2-((4-cyano-1,3,5-triazin-2-yl)amino)-7-azaspiro[3.5]nonane-7-carboxylate

To a solution of tert-butyl 2-[(4-chloro-1,3,5-triazin-2-yl)amino]-7-azaspiro[3.5]nonane-7-carboxylate (1.51 g, 4.27 mmol) and KCN (610 mg, 9.37 mmol) in DMSO (35 mL) was added DABCO (95.7 mg, 853 μmol). The mixture was stirred at 25° C. for 1 hr. The reaction mixture was poured into water (200 mL). The resulting mixture was extracted with EtOAc (100 mL×2). The combined organic layers were washed with brine (100 mL) and dried over anhydrous Na₂SO₄, filtered and concentrated to dryness. The residue was purified by silica gel flash chromatography (Eluent of 0˜30% EtOAc in petroleum ether) to give tert-butyl 2-[(4-cyano-1,3,5-triazin-2-yl)amino]-7-azaspiro[3.5]nonane-7-carboxylate (1.2 g, yield: 82%) as a white solid. MS: m/z=289.1 [M−56+H]⁺. ¹H NMR (400 MHz, Chloroform-d) δ 8.63-8.51 (m, 1H), 5.94-5.84 (m, 1H), 4.50-4.43 (m, 1H), 3.41-3.36 (m, 2H), 3.32-3.28 (m, 2H), 2.47-2.40 (m, 2H), 1.76-1.70 (m, 2H), 1.65-1.61 (m, 2H), 1.56-1.52 (m, 2H), 1.45 (s, 9H).

### Step 3: 4-(7-Azaspiro[3.5]nonan-2-ylamino)-1,3,5-triazine-2-carbonitrile

To a solution of tert-butyl 2-[(4-cyano-1,3,5-triazin-2-yl)amino]-7-azaspiro[3.5]nonane-7-carboxylate (980 mg, 2.85 mmol) in CH₂Cl₂ (10 mL) was added TFA (1.95 g, 17.1 mmol). The mixture was stirred at 25° C. for 1 hr. The reaction mixture was concentrated under reduced pressure. The crude 4-(7-azaspiro[3.5]nonan-2-ylamino)-1,3,5-triazine-2-carbonitrile (Intermediate 65, 1.02 g TFA salt, yield: 100%) was used in the next step without further purification. MS: m/z=245.1 [M+H]⁺.

### Intermediate 66: Methyl 4-(2-(2-aminopyridin-3-yl)-5-chloro-3H-imidazo[4,5-b]pyridin-3-yl)benzoate

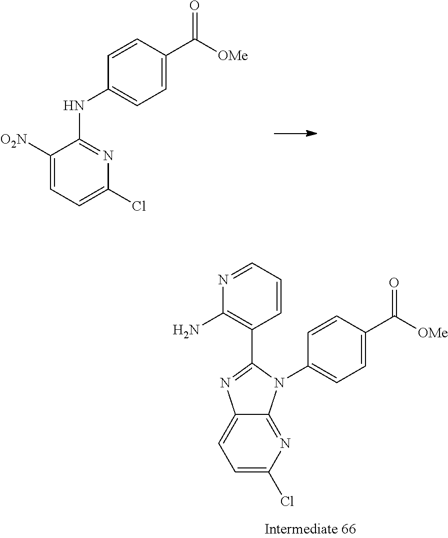 [SMILES: COC(=O)c1ccc(Nc2nc(Cl)ccc2[N+](=O)[O-])cc1 ; COC(=O)c1ccc(-n2c(-c3cccnc3N)nc3ccc(Cl)nc32)cc1]

To a solution of methyl 4-((6-chloro-3-nitropyridin-2-yl)amino)benzoate (refer to Intermediate 13 for detail procedures, 5.0 g, 16.3 mmol) and 2-aminonicotinaldehyde (2.18 g, 81.8 mmol) in DMSO (150 mL) was added Na₂S₂O₄ (5.66 g, 32.5 mmol). The mixture was stirred at 105° C. for 12 hr. The reaction mixture was diluted with water (300 mL) and extracted with EtOAc (300 mL×3). The combined organic layers were washed with brine (100 mL), dried over Na₂SO₄, filtered and concentrated under reduced pressure. The residue was purified by silica gel flash chromatography (Eluent of 0˜100% EtOAc in petroleum ether) to give methyl 4-(2-(2-aminopyridin-3-yl)-5-chloro-3H-imidazo[4,5-b]pyridin-3-yl)benzoate (Intermediate 66, 700 mg, yield: 11.3%) as a yellow solid. MS: m/z=380.0 [M+H]⁺. ¹H NMR (400 MHz, Dimethylsulfoxide-d₆) δ 8.28 (d, J=8.4 Hz, 1H), 8.09 (d, J=8.4 Hz, 2H), 8.01 (dd, J=4.4, 1.6 Hz, 1H), 7.60 (d, J=8.4 Hz, 2H), 7.48 (d, J=8.4 Hz, 1H), 7.23 (dd, J=7.6, 1.6 Hz, 1H), 6.78 (br s, 2H), 6.45 (dd, J=7.6, 4.8 Hz, 1H), 3.89 (s, 3H).

### Intermediate 67: 3-(3-(4-((4-Aminopiperidin-1-yl)methyl)phenyl)-5-methyl-3H-imidazo[4,5-b]pyridin-2-yl)pyridin-2-amine

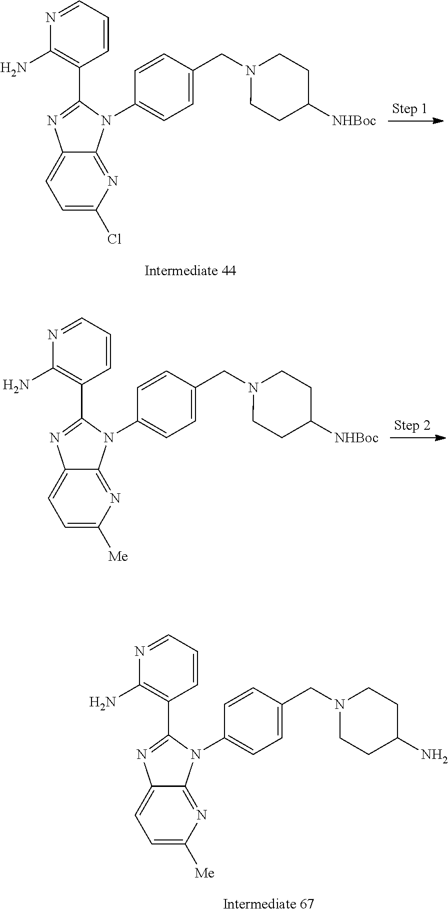 [SMILES: CC(C)(C)OC(=O)NC1CCN(Cc2ccc(-n3c(-c4cccnc4N)nc4ccc(Cl)nc43)cc2)CC1 ; Cc1ccc2nc(-c3cccnc3N)n(-c3ccc(CN4CCC(NC(=O)OC(C)(C)C)CC4)cc3)c2n1 ; Cc1ccc2nc(-c3cccnc3N)n(-c3ccc(CN4CCC(N)CC4)cc3)c2n1]

### Step 1: tert-Butyl (1-(4-(2-(2-aminopyridin-3-yl)-5-methyl-3H-imidazo[4,5-b]pyridin-3-yl)benzyl)piperidin-4-yl)carbamate

A mixture of Intermediate 44 (300 mg, 562 μmol), methylboronic acid (47 mg, 786 μmol), Cs₂CO₃ (366 mg, 1.12 mmol), and Pd(dppf)Cl₂ (41 mg, 56.2 μmol) in 1,4-dioxane (2 mL) and H₂O (0.5 mL) was degassed and purged with N₂ three times, and then the mixture was stirred at 100° C. for 16 hr under N₂ atmosphere. The reaction mixture was poured into H₂O (10 mL) and extracted with EtOAc (15 mL×2). The combined organic layers were dried over anhydrous Na₂SO₄, filtered and concentrated under reduced pressure. The residue was purified by silica gel flash chromatography (Eluent of 0˜86% EtOAc in petroleum ether) to give tert-butyl (1-(4-(2-(2-aminopyridin-3-yl)-5-methyl-3H-imidazo[4,5-b]pyridin-3-yl)benzyl)piperidin-4-yl)carbamate (210 mg, yield: 73%) as a yellow solid. MS: m/z=514.2 [M+H]⁺.

### Step 2: 3-(3-(4-((4-Aminopiperidin-1-yl)methyl)phenyl)-5-methyl-3H-imidazo[4,5-b]pyridin-2-yl)pyridin-2-amine

To a solution of ter-butyl (1-(4-(2-(2-aminopyridin-3-yl)-5-methyl-3H-imidazo[4,5-b]pyridin-3-yl)benzyl)piperidin-4-yl)carbamate (60 mg, 117 μmol) in CH₂Cl₂ (2 mL) was added 4 M HCl in 1,4-dioxane (1.5 mL). The mixture was stirred at 25° C. for 1 hr. The reaction was concentrated under reduced pressure to give crude product. The crude was triturated with CH₂Cl₂ (10 mL) at 25° C. for 30 min. 3-(3-(4-((4-Aminopiperidin-1-yl)methyl)phenyl)-5-methyl-3H-imidazo[4,5-b]pyridin-2-yl)pyridin-2-amine (Intermediate 67, 43.6 mg HCl salt, yield: 83%) was obtained as a yellow solid. MS: m/z=414.0 [M+H]⁺. ¹H NMR (400 MHz, Methanol-d₄) δ 8.17 (d, J=8.4 Hz, 1H), 7.99 (d, J=5.6 Hz, 1H), 7.88 (d, J=7.6 Hz, 2H), 7.81 (d, J=7.2 Hz, 1H), 7.63 (d, J=7.6 Hz, 2H), 7.38 (d, J=8.4 Hz, 1H), 6.88-6.82 (m, 1H), 3.70-3.64 (m, 2H), 3.59-3.54 (m, 1H), 3.29-3.17 (m, 2H), 2.61 (s, 3H), 2.32-2.29 (m, 2H), 2.20-2.06 (m, 2H), 1.33-1.27 (m, 2H).

### Intermediate 68: 3-(5-(2-Fluorophenyl)-3-(4-(piperazin-1-ylmethyl)phenyl)-3H-imidazo[4,5-b]pyridin-2-yl)pyridin-2-amine hydrochloride

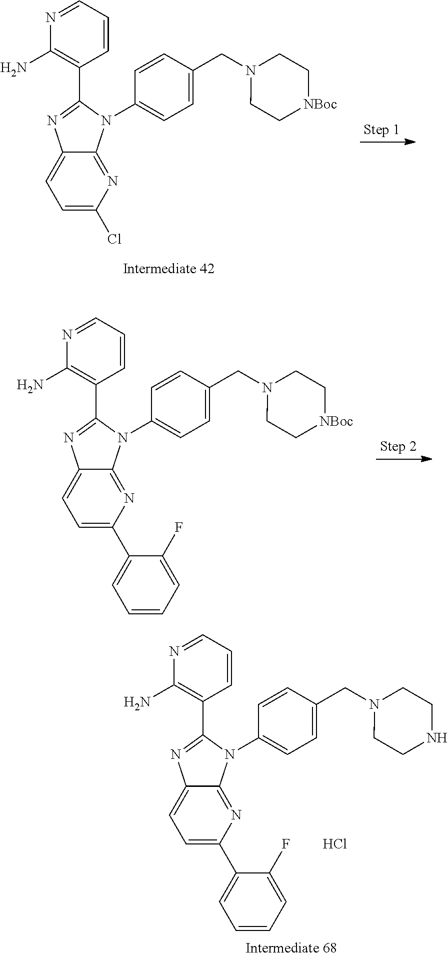

### Step 1: tert-Butyl 4-(4-(2-(2-aminopyridin-3-yl)-5-(2-fluorophenyl)-3H-imidazo[4,5-b]pyridin-3-yl)benzyl)piperazine-1-carboxylate

To a solution of Intermediate 42 (500 mg, 961 μmol) and (2-fluorophenyl)boronic acid (134 mg, 961 μmol) in 1,4-dioxane (10 mL) and H₂O (2 mL) was added Cs₂CO₃ (940 mg, 2.88 mmol) and Pd(dppf)Cl₂ (70.4 mg, 96.1 μmol). The mixture was degassed and purged with N₂ three times, the resulting mixture was stirred at 80° C. for 16 hr under N₂ atmosphere. Then the reaction mixture was diluted with H₂O (50 mL) and extracted with CH₂Cl₂ (50 mL×2). The combined organic layers were dried over Na₂SO₄, filtered and concentrated under reduced pressure. The crude product was purified by silica gel flash chromatography (Eluent of 30-80% EtOAc in petroleum ether) to give tert-Butyl 4-(4-(2-(2-aminopyridin-3-yl)-5-(2-fluorophenyl)-3H-imidazo[4,5-b]pyridin-3-yl)benzyl)piperazine-1-carboxylate (500 mg, yield: 87%) as a yellow solid. MS: m/z=580.2 [M+H]⁺. ¹H NMR (400 MHz, Dimethylsulfoxide-d₆) δ 8.30 (d, J=8.4 Hz, 1H), 8.00 (dd, J=4.8, 1.6 Hz, 1H), 7.90-7.70 (m, 2H), 7.56-7.40 (m, 5H), 7.37-7.26 (m, 2H), 7.16 (dd, J=8.0, 2.0 Hz, 1H), 6.99 (br s, 2H), 6.39 (dd, J=7.6, 4.8 Hz, 1H), 3.57 (s, 2H), 3.36-3.33 (m, 4H), 2.42-2.30 (m, 4H), 1.40 (s, 9H).

### Step 2: 3-(5-(2-Fluorophenyl)-3-(4-(piperazin-1-ylmethyl)phenyl)-3H-imidazo[4,5-b]pyridin-2-yl)pyridin-2-amine hydrochloride

To a solution of tert-butyl 4-(4-(2-(2-aminopyridin-3-yl)-5-(2-fluorophenyl)-3H-imidazo[4,5-b]pyridin-3-yl)benzyl)piperazine-1-carboxylate (500 mg, 863 μmol) in 1,4-dioxane (5 mL) was added HCl/1,4-dioxane (4 M, 5 mL). The mixture was stirred at 25° C. for 0.5 hr. The reaction mixture was filtered and concentrated under reduced pressure to give 3-(5-(2-fluorophenyl)-3-(4-(piperazin-1-ylmethyl)phenyl)-3H-imidazo[4,5-b]pyridin-2-yl)pyridin-2-amine (Intermediate 68, 400 mg, yield: 92%, HCl salt) as a yellow solid. MS: m/z=480.2 [M+H]⁺. ¹H NMR (400 MHz, Dimethylsulfoxide-d₆) δ 12.82-11.99 (m, 1H), 10.20-9.62 (m, 2H), 8.53-8.43 (m, 1H), 8.40 (d, J=8.0 Hz, 1H), 8.15 (dd, J=6.0, 1.6 Hz, 1H), 7.91-7.81 (m, 5H), 7.67 (d, J=8.4 Hz, 2H), 7.52-7.46 (m, 1H), 7.39-7.30 (m, 2H), 6.98 (dd, J=7.6, 6.4 Hz, 1H), 4.48 (s, 2H), 3.53-3.48 (m, 4H), 2.50 (m, 4H). ¹⁹F NMR (400 MHz, Dimethylsulfoxide-d₆) 6-117.18.

### Intermediate 69: 2-(2-(2-Aminopyridin-3-yl)-3-(4-(piperazin-1-ylmethyl)phenyl)-3H-imidazo[4,5-b]pyridin-5-yl)benzonitrile

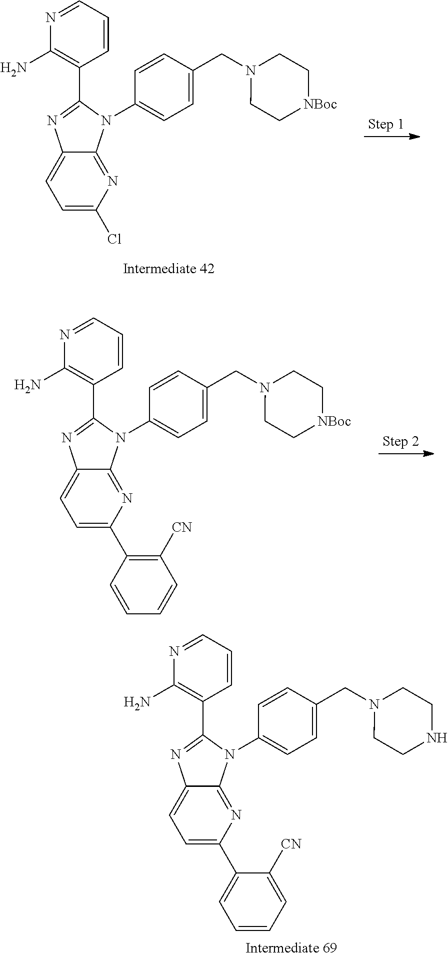

### Step 1: tert-Butyl 4-(4-(2-(2-aminopyridin-3-yl)-5-(2-cyanophenyl)-3H-imidazo[4,5-b]pyridin-3-yl)benzyl)piperazine-1-carboxylate

A mixture of Intermediate 42 (400 mg, 770 μmol), (2-cyanophenyl)boronic acid (124 mg, 846 μmol), K₃PO₄ (327 mg, 1.54 mmol), cataCXiumAPdG₃ (280 mg, 385 μmol), and tricyclohexylphosphane (22 mg, 76.9 μmol) in DMF (5 mL) was degassed and purged with N₂ three times, and then the mixture was stirred at 120° C. for 16 hr under N₂ atmosphere. The reaction mixture was filtered at 25° C., and then diluted with H₂O (20 mL) and extracted with CH₂Cl₂ (20 mL×3). The combined organic layers were washed with brine (30 mL), dried over Na₂SO₄, filtered and concentrated under reduced pressure. After purified by silica gel flash chromatography (Eluent of 20˜80% EtOAc in Petroleum ether), tert-butyl 4-(4-(2-(2-aminopyridin-3-yl)-5-(2-cyanophenyl)-3H-imidazo[4,5-b]pyridin-3-yl)benzyl)piperazine-1-carboxylate (400 mg, yield: 80%) was obtained as a yellow solid. MS: m/z=587.2 [M+H]⁺.

### Step 2: 2-(2-(2-Aminopyridin-3-yl)-3-(4-(piperazin-1-ylmethyl)phenyl)-3H-imidazo[4,5-b]pyridin-5-yl)benzonitrile

To a solution of ter-butyl 4-(4-(2-(2-aminopyridin-3-yl)-5-(2-cyanophenyl)-3H-imidazo[4,5-b]pyridin-3-yl)benzyl)piperazine-1-carboxylate (400 mg, 682 μmol) in 1,4-dioxane (5 mL) was added 4 M HCl in 1,4-dioxane (3 mL) at 25° C. The mixture was stirred at 25° C. for 16 hr. The reaction mixture was then filtered and the collected solid was washed with 1,4-dioxane (5 mL×2) and dried in vacuo to give 2-(2-(2-aminopyridin-3-yl)-3-(4-(piperazin-1-ylmethyl)phenyl)-3H-imidazo[4,5-b]pyridin-5-yl)benzonitrile (Intermediate 69, 268 mg HCl salt, yield: 75%) as a yellow solid. MS: m/z=487.2 [M+H]⁺. ¹H NMR (400 MHz, Dimethylsulfoxide-d₆) δ 8.38 (d, J=8.0 Hz, 1H), 8.03-7.99 (m, J H), 7.97-7.92 (m, 1H), 7.90-7.77 (m, 3H), 7.64-7.58 (m, 1H), 7.50-7.37 (m, 5H), 7.19 (dd, J=7.6, 2.0 Hz, 1H), 6.98 (br s, 2H), 6.40 (dd, J=7.6, 4.8 Hz, 1H), 3.50 (s, 2H), 2.80-2.70 (m, 4H), 2.37-2.32 (m, 4H).

### Intermediate 70: 4-(2-(2-aminopyridin-3-yl)-3-(4-(piperazin-1-ylmethyl)phenyl)-3H-imidazo[4,5-b]pyridin-5-yl)-1,4-diazepan-2-one

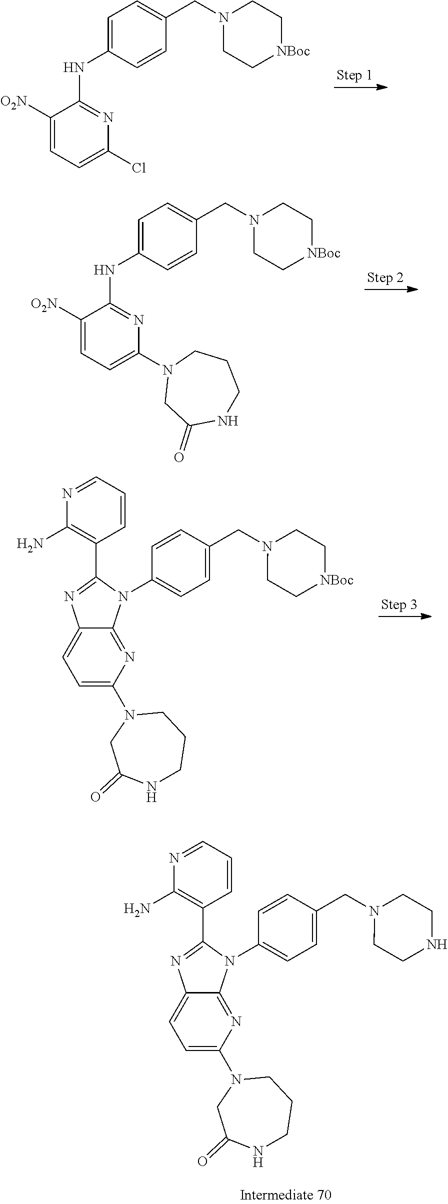 [SMILES: CC(C)(C)OC(=O)N1CCN(Cc2ccc(Nc3nc(Cl)ccc3[N+](=O)[O-])cc2)CC1 ; CC(C)(C)OC(=O)N1CCN(Cc2ccc(Nc3nc(N4CCCNC(=O)C4)ccc3[N+](=O)[O-])cc2)CC1 ; CC(C)(C)OC(=O)N1CCN(Cc2ccc(-n3c(-c4cccnc4N)nc4ccc(N5CCCNC(=O)C5)nc43)cc2)CC1 ; Nc1ncccc1-c1nc2ccc(N3CCCNC(=O)C3)nc2n1-c1ccc(CN2CCNCC2)cc1]

### Step 1: terl-Butyl 4-(4-((3-nitro-6-(3-oxo-1,4-diazepan-1-yl)pyridin-2-yl)amino)benzyl)piperazine-1-carboxylate

To a solution of tert-butyl 4-(4-((6-chloro-3-nitropyridin-2-yl)amino)benzyl)piperazine-1-carboxylate (refer to Intermediate 10 for detail procedures, 1.5 g, 3.35 mmol) and 1,4-diazepan-2-one (382 mg, 3.35 mmol) in ACN (20 mL) was added DIEA (866 mg, 6.7 mmol). The mixture was stirred at 90° C. for 4 hr. The reaction was concentrated under reduced pressure to give tert-butyl 4-(4-((3-nitro-6-(3-oxo-1,4-diazepan-1-yl)pyridin-2-yl)amino)benzyl)piperazine-1-carboxylate (1.8 g, yield: 92%) as a yellow solid. MS: m/z=526.1 [M+H]⁺.

### Step 2: tert-Butyl 4-(4-(2-(2-aminopyridin-3-yl)-5-(3-oxo-1,4-diazepan-1-yl)-3H-imidazo[4,5-b]pyridin-3-yl)benzyl)piperazine-1-carboxylate

To a solution of tert-butyl 4-(4-((3-nitro-6-(3-oxo-1,4-diazepan-1-yl)pyridin-2-yl)amino)benzyl)piperazine-1-carboxylate (1.8 g, 3.42 mmol) in DMSO (20 mL) was added 2-aminonicotinaldehyde (502 mg, 4.11 mmol) and Na₂S₂O₄ (2.74 g, 13.7 mmol, 87% purity). The mixture was stirred at 100° C. for 8 hr. The reaction mixture was quenched with H₂O (30 mL) at 25° C., and extracted with CH₂Cl₂ (50 mL×3). The combined organic layers were washed with brine (50 mL), dried over Na₂SO₄, filtered and concentrated under reduced pressure to give tert-butyl 4-(4-(2-(2-aminopyridin-3-yl)-5-(3-oxo-1,4-diazepan-1-yl)-3H-imidazo[4,5-b]pyridin-3-yl)benzyl)piperazine-1-carboxylate (1.3 g, purity: 49%, yield: 30%) as a yellow solid. MS: m/z=598.3 [M+H]⁺, 498.2 [M−Boc+H]⁺

### Step 3: 4-(2-(2-Aminopyridin-3-yl)-3-(4-(piperazin-1-ylmethyl)phenyl)-3H-imidazo[4,5-b]pyridin-5-yl)-1,4-diazepan-2-one

To a solution of tert-butyl 4-(4-(2-(2-aminopyridin-3-yl)-5-(3-oxo-1,4-diazepan-1-yl)-3H-imidazo[4,5-b]pyridin-3-yl)benzyl)piperazine-1-carboxylate (1.2 g, 2.01 mmol) in 4 M HCl in 1,4-dioxane (10 mL) at 25° C. The mixture was stirred at 25° C. for 0.5 hr. The reaction was concentrated under reduced pressure to give crude product (1 g, yield: 54%). The crude (100 mg) was purified by prep-HPLC (column: Phenomenex C18 150*25 mm*10 μm; mobile phase: [water (NH₄HCO₃)-ACN]; gradient: B: 9%-39%, 10 min) to give 4-(2-(2-aminopyridin-3-yl)-3-(4-(piperazin-1-ylmethyl)phenyl)-3H-imidazo[4,5-b]pyridin-5-yl)-1,4-diazepan-2-one (Intermediate 70, 25.1 mg) as a yellow solid. MS: m/z=498.3 [M+H]⁺. ¹H NMR (400 MHz, Dimethylsulfoxide-d₆) 5.8.01-7.89 (m, 2H), 7.52-7.42 (m, 1H), 7.40 (d, J=8.4 Hz, 2H), 7.31 (d, J=8.4 Hz, 2H), 7.00 (dd, J=7.6, 1.6 Hz, 1H), 6.98 (br s, 1H), 6.80 (d, J=8.8 Hz, 1H), 6.32 (dd, J=7.6, 4.8 Hz, 1H), 4.17 (s, 2H), 3.85-3.77 (m, 2H), 3.49 (s, 2H), 3.19-3.15 (m, 2H), 2.70 (t, J=4.8 Hz, 4H), 2.35-2.27 (m, 4H), 1.68-1.61 (m, 2H).

### Intermediate 71: 4-(3,8-Diazabicyclo[3.2.1]octan-3-yl)-1,3,5-triazine-2-carbonitrile

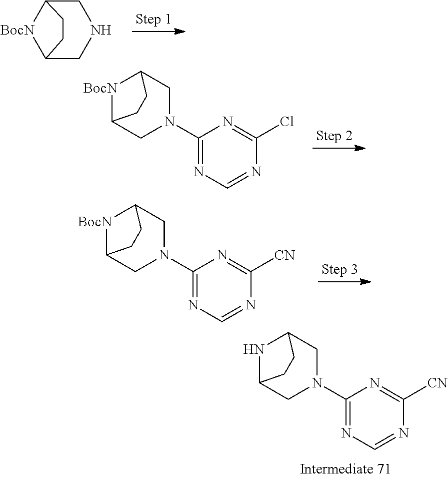

### Step 1: tert-Butyl 3-(4-chloro-1,3,5-triazin-2-yl)-3,8-diazabicyclo[3.2.1]octane-8-carboxylate

To a solution of tert-butyl 3,8-diazabicyclo[3.2.1]octane-8-carboxylate (5 g, 23.6 mmol) and 2,4-dichloro-1,3,5-triazine (3.89 g, 25.9 mmol) in THF (100 mL) was added DIEA (9.13 g, 70.7 mmol). The resulting mixture was stirred at 0° C. for 1 hr. Then the reaction mixture was diluted with H₂O (100 mL) and extracted with CH₂Cl₂ (100 mL×2). The combined organic layers were dried over Na₂SO₄, filtered and concentrated under reduced pressure. After purified by silica gel flash chromatography (Eluent of 20˜30% EtOAc in petroleum ether), tert-butyl 3-(4-chloro-1,3,5-triazin-2-yl)-3,8-diazabicyclo[3.2.1]octane-8-carboxylate (4 g, yield: 51%) was obtained as a white solid. MS: m/z=326.2 [M+H]⁺. ¹H NMR (400 MHz, Dimethylsulfoxide-d₆) δ 8.50 (s, 1H), 4.42-4.35 (m, 1H), 4.27-4.19 (m, 3H), 3.14-3.08 (m, 2H), 1.85-1.77 (m, 2H), 1.56-1.50 (m, 2H), 1.43 (s, 9H).

### Step 2: tert-Butyl 3-(4-cyano-1,3,5-triazin-2-yl)-3,8-diazabicyclo[3.2.1]octane-8-carboxylate

To a solution of tert-butyl 3-(4-chloro-1,3,5-triazin-2-yl)-3,8-diazabicyclo[3.2.1]octane-8-carboxylate (1.9 g, 5.83 mmol) in DMSO (20 mL) were added KCN (790 mg, 12.1 mmol) and DABCO (131 mg, 1.17 mmol). The mixture was stirred at 25° C. for 16 hr. The reaction mixture was poured into H₂O (100 mL). The resulting mixture was extracted with EtOAc (100 mL×2). The combined organic layers were washed with brine (100 mL), dried over Na₂SO₄, filtered and concentrated under reduced pressure. After purified by silica gel flash chromatography (Eluent of 20˜30% EtOAc in petroleum ether), tert-butyl 3-(4-cyano-1,3,5-triazin-2-yl)-3,8-diazabicyclo[3.2.1]octane-8-carboxylate (650 mg, yield: 30%) was obtained as a yellow solid. MS: m/z=317.2 [M+H]⁺.

### Step 3: 4-(3,8-Diazabicyclo[3.2.1]octan-3-yl)-1,3,5-triazine-2-carbonitrile

To a solution of tert-butyl 3-(4-cyano-1,3,5-triazin-2-yl)-3,8-diazabicyclo[3.2.1]octane-8-carboxylate (630 mg, 1.99 mmol) in CH₂Cl₂ (10 mL) was added TFA (681 mg, 5.97 mmol). The mixture was stirred at 25° C. for 0.5 hr. The reaction mixture was filtered and concentrated under reduced pressure, 4-(3,8-diazabicyclo[3.2.1]octan-3-yl)-1,3,5-triazine-2-carbonitrile (Intermediate 71, 430 mg TFA salt, yield: 52%) was obtained as a yellow oil. MS: m/z=217.1 [M+H]⁺.

### Intermediate 72: 3-(3-(4-(Chloromethyl)phenyl)-5-isopropyl-3H-imidazo[4,5-h]pyridin-2-yl)pyridin-2-amine

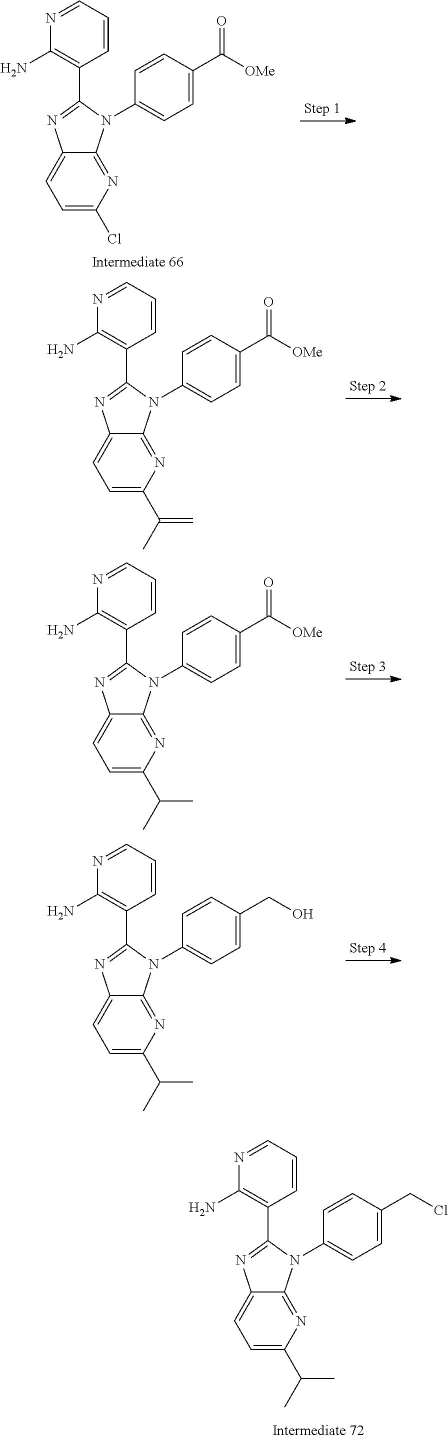

### Step 1: Methyl 4-(2-(2-aminopyridin-3-yl)-5-(prop-1-en-2-yl)-3H-imidazo[4,5-b]pyridin-3-yl)benzoate

A mixture of Intermediate 66 (660 mg, 1.74 mmol), 4,4,5,5-tetramethyl-2-(prop-1-en-2-yl)-1,3,2-dioxaborolane (321 mg, 1.91 mmol), Pd(dppf)Cl₂ (127 mg, 173 μmol) and Cs₂CO₃ (1.13 g, 3.48 mmol) in 1,4-dioxane (10 mL) and H₂O (1 mL) was degassed and purged with N₂ three times. The mixture was stirred at 100° C. for 3 hr. The reaction mixture was diluted with H₂O (50 mL) and extracted with CH₂Cl₂ (50 mL×2). The combined organic layers were dried over Na₂SO₄, filtered and concentrated under reduced pressure. After purified by silica gel flash chromatography (eluent of 50% EtOAc in petroleum ether), methyl 4-(2-(2-aminopyridin-3-yl)-5-(prop-1-en-2-yl)-3H-imidazo[4,5-b]pyridin-3-yl)benzoate (530 mg, yield: 72%) was obtained as a yellow solid. MS: m/z=386.0 [M+H]⁺.

### Step 2: Methyl 4-(2-(2-aminopyridin-3-yl)-5-isopropyl-3H-imidazo[4,5-b]pyridin-3-yl)benzoate A mixture of methyl 4-(2-(2-aminopyridin-3-yl)-5-(prop-1-en-2-yl)-3H-imidazo[4,5-b]pyridin-3-yl)benzoate

(530 mg, 1.38 mmol) in MeOH (20 mL) was added Pd/C (150 mg, 2.75 mmol, 10% purity), the mixture was stirred at 50° C. and 50 psi under H₂ atmosphere for 24 hr. The reaction mixture was cooled to 25° C., then filtered to get the liquor and concentrated under reduced pressure to give methyl 4-(2-(2-aminopyridin-3-yl)-5-isopropyl-3H-imidazo[4,5-b]pyridin-3-yl)benzoate (500 mg, yield: 94%) as a green solid. MS: m/z=388.1 [M+H]+. ¹H NMR (400 MHz, Dimethylsulfoxide-d₆) δ 8.13 (d, J=8.4 Hz, 1H), 8.08 (d, J=8.4 Hz, 2H), 8.00 (dd, J=4.8, 1.6 Hz, 1H), 7.58 (d, J=8.4 Hz, 2H), 7.33 (d, J=8.0 Hz, 1H), 7.17 (dd, J=7.6, 1.6 Hz, 1H), 6.78 (br s, 2H), 6.43 (dd, J=7.6, 4.8 Hz, 1H), 3.88 (s, 3H), 3.12-3.04 (m, 1H), 1.23 (d, J=6.8 Hz, 6H).

### Step 3: (4-(2-(2-Aminopyridin-3-yl)-5-isopropyl-3H-imidazo[4,5-b]pyridin-3-yl)phenyl)methanol

A mixture of methyl methyl 4-(2-(2-aminopyridin-3-yl)-5-isopropyl-3H-imidazo[4,5-b]pyridin-3-yl)benzoate (500 mg, 1.29 mmol) in THF (20 mL) was added LiAlH₄ (2.5 M, 800 L) at 0° C. dropwise, the mixture was stirred at 25° C. for 1 hr. The reaction mixture was quenched with Na₂SO₄·10H₂O (3 g) at 0° C., and then filtered and concentrated under reduced pressure to give (4-(2-(2-aminopyridin-3-yl)-5-isopropyl-3H-imidazo[4,5-b]pyridin-3-yl)phenyl)methanol (480 mg, crude) as a yellow solid. MS: m/z=360.0 [M+H]⁺.

### Step 4: 3-(3-(4-(Chloromethyl)phenyl)-5-isopropyl-3H-imidazo[4,5-b]pyridin-2-yl)pyridin-2-amine

A mixture of (4-(2-(2-aminopyridin-3-yl)-5-isopropyl-3H-imidazo[4,5-b]pyridin-3-yl)phenyl)methanol (480 mg, 1.34 mmol) in CH₂Cl₂ (10 mL) was added SOCl₂ (953 mg, 8.0 mmol), the mixture was stirred at 40° C. for 1 hr. The reaction mixture was filtered and the filter cake was washed with CH₂Cl₂ (5 mL) to give 3-(3-(4-(chloromethyl)phenyl)-5-isopropyl-3H-imidazo[4,5-b]pyridin-2-yl)pyridin-2-amine (Intermediate 72, 220 mg, yield: 41%) as a yellow solid. MS: m/z=378.0 [M+H]⁺.

### Intermediate 73: 3-(3-(4-(Chloromethyl)phenyl)-5-methoxy-3H-imidazo[4,5-b]pyridin-2-yl)pyridin-2-amine

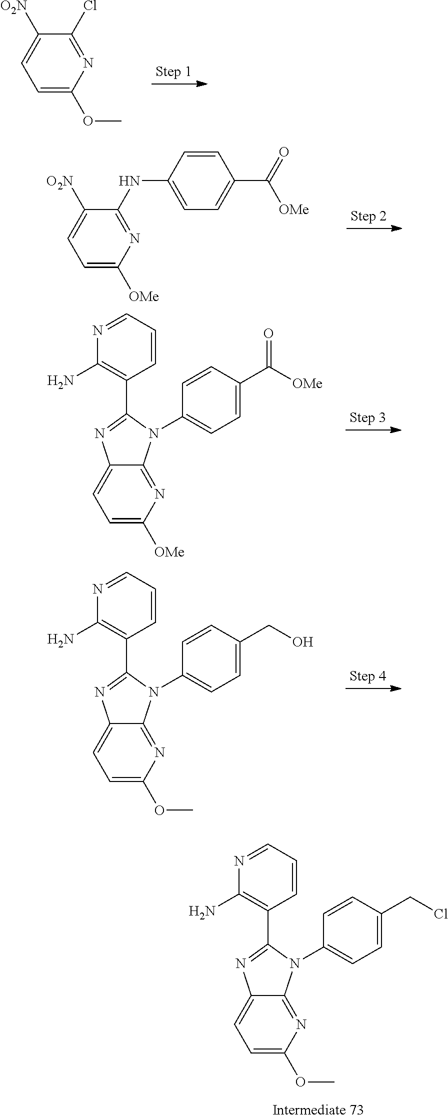

### Step 1: Methyl 4-((6-methoxy-3-nitropyridin-2-yl)amino)benzoate

To a solution of 2-chloro-6-methoxy-3-nitropyridine (10 g, 53.0 mmol) and methyl 4-aminobenzoate (7.62 g, 50.4 mmol) in DIEA (50 mL). The mixture was stirred at 130° C. for 4 hr. The mixture was diluted with EtOAc (50 mL) and H₂O (30 mL). The separated organic layer was washed with H₂O (60 mL) and dried over Na₂SO₄, filtered and concentrated. After purified by silica gel flash chromatography (Eluent of 0˜10% EtOAc in petroleum ether), methyl 4-((6-methoxy-3-nitropyridin-2-yl)amino)benzoate (5.12 g, yield. 26%) was obtained as a light-yellow solid. MS: m/z=304.0 [M+H]⁺. ¹H NMR (400 MHz, Chloroform-d) δ 10.79 (s, 1H), 8.48-8.41 (m, 1H), 8.06 (d, J=8.4 Hz, 2H), 7.78 (d, J=8.0 Hz, 2H), 6.31 (dd, J=9.2, 0.8 Hz, 1H), 4.01 (s, 3H), 3.92 (s, 3H).

### Step 2: Methyl 4-(2-(2-aminopyridin-3-yl)-5-methoxy-3H-imidazo[4,5-b]pyridin-3-yl)benzoate

To a solution of methyl 4-((6-methoxy-3-nitropyridin-2-yl)amino)benzoate (5.12 g, 16.9 mmol) in DMSO (100 mL) was added Na₂S₂O₄ (13.5 g, 67.5 mmol, 87% purity) and 2-aminonicotinaldehyde (2.47 g, 20.3 mmol). The mixture was stirred at 100° C. for 16 hr. The reaction mixture was quenched with H₂O (25 mL) at 25° C., and then extracted with CH₂Cl₂ (50 mL×3). The combined organic layers were washed with brine (50 mL), dried over Na₂SO₄, filtered and concentrated under reduced pressure. After purified by silica gel flash chromatography (Eluent of 0˜35% EtOAc in petroleum ether), methyl 4-(2-(2-aminopyridin-3-yl)-5-methoxy-3H-imidazo[4,5-b]pyridin-3-yl)benzoate (2.0 g, yield: 28%) was obtained as a light-yellow solid. MS: m/z=376.0 [M+H]⁺. ¹H NMR (400 MHz, Chloroform-d) δ 8.16 (d, J=8.4 Hz, 2H), 8.04 (dd, J=5.2, 2.0 Hz, 1H), 7.97 (d, J=8.8 Hz, 1H), 7.47 (d, J=8.8 Hz, 2H), 6.99 (dd, J=7.6, 1.6 Hz, 1H), 6.77 (d, J=8.4 Hz, 1H), 6.55 (br s, 2H), 6.37 (dd, J=8.0, 5.2 Hz, 1H), 3.96 (s, 3H), 3.86 (s, 3H).

### Step 3: (4-(2-(2-Aminopyridin-3-yl)-5-methoxy-3H-imidazo[4,5-b]pyridin-3-yl)phenyl)methanol

A mixture of methyl 4-(2-(2-aminopyridin-3-yl)-5-methoxy-3H-imidazo[4,5-b]pyridin-3-yl)benzoate (2.0 g, 5.33 mmol) in THF (130 mL) was added LiAlH₄ (2.5 M, 2.77 mL) at 0° C. dropwise, the mixture was stirred at 25° C. for 2 hr. The reaction mixture was quenched with Na₂SO₄·10H₂O (3 g) at 0° C., and then filtered and concentrated under reduced pressure to give (4-(2-(2-aminopyridin-3-yl)-5-methoxy-3H-imidazo[4,5-b]pyridin-3-yl)phenyl)methanol (1.8 g, crude) as a yellow solid. MS: m/z=348.0 [M+H]⁺.

### Step 4: 3-(3-(4-(Chloromethyl)phenyl)-5-methoxy-3H-imidazo[4,5-b]pyridin-2-yl)pyridin-2-amine

To a solution of (4-(2-(2-aminopyridin-3-yl)-5-methoxy-3H-imidazo[4,5-b]pyridin-3-yl)phenyl)methanol (1.8 g, 5.18 mmol) in CH₂Cl₂ (20 mL) was added SOCl₂ (1.23 g, 10.4 mmol). The mixture was stirred at 40° C. for 2 hr. The residue was poured into water (200 mL). The resulting mixture was washed with CH₂Cl₂ (10 mL×2), then aqueous phase was adjusted pH to around 8 by NaHCO₃. The resulting mixture was extracted with CH₂Cl₂ (100 mL). The combined organic layers were dried over anhydrous Na₂SO₄, filtered and concentrated to dryness. After purified by silica gel flash chromatography (Eluent of 0˜35% EtOAc in petroleum ether), 3-(3-(4-(chloromethyl)phenyl)-5-methoxy-3H-imidazo[4,5-b]pyridin-2-yl)pyridin-2-amine (Intermediate 73, 1.3 g, yield: 65%) was obtained as a light-yellow solid. MS: m/z=366.0 [M+H]⁺.

### Intermediate 74: 2-(2-Aminopyridin-3-yl)-3-(4-(chloromethyl)phenyl)-3H-imidazo[4,5-b]pyridine-5-carboxamide

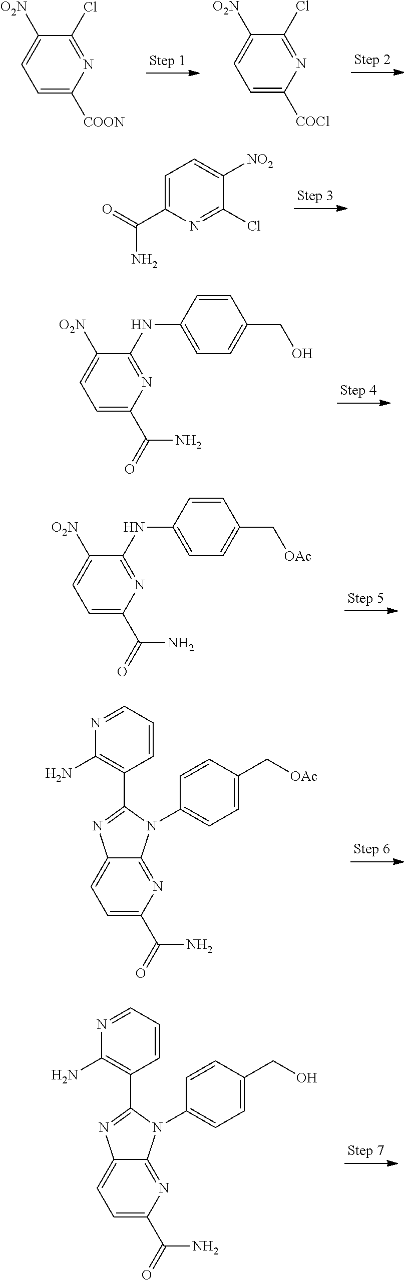

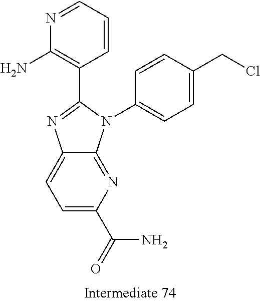 [SMILES: NC(=O)c1ccc2nc(-c3cccnc3N)n(-c3ccc(CCl)cc3)c2n1]

### Step 1: 6-Chloro-5-nitropicolinoyl chloride

A solution of 6-chloro-5-nitropicolinic acid (5 g, 24.7 mmol) in SOC₂ (20 mL) was stirred at 80° C. for 16 hr. The reaction mixture was filtered and concentrated under reduced pressure to give 6-chloro-5-nitropicolinoyl chloride (5.4 g, yield: 97%) as a yellow solid, which was directly used to the next step.

### Step 2: 6-Chloro-5-nitropicolinamide

To a solution of 6-chloro-5-nitropicolinoyl chloride (5 g, 22.6 mmol) in THF (50 mL) was added NH₃·H₂O (9.51 g, 67.9 mmol) at 0° C. The mixture was stirred at 0° C. for 1 hr. Then the reaction mixture was diluted with H₂O (100 mL) and extracted with EtOAc (100 mL×2). The combined organic layers were dried over Na₂SO₄, filtered and concentrated under reduced pressure to give 6-chloro-5-nitropicolinamide (3.3 g, yield: 65%) as a yellow solid. MS: m/z=201.9 [M+H]⁺.

### Step 3: 6-((4-(Hydroxymethyl)phenyl)amino)-5-nitropicolinamide

To a solution of 6-chloro-5-nitropicolinamide (3.3 g, 16.4 mmol) in DMSO (20 mL) were added (4-aminophenyl)methanol (2.02 g, 16.4 mmol) and DIEA (6.35 g, 49.1 mmol). The mixture was stirred at 100° C. for 16 hr. The reaction mixture was diluted with H₂O (100 mL) and extracted with CH₂Cl₂ (100 mL×2). The combined organic layers were washed with brine (100 mL×2), dried over Na₂SO₄, filtered and concentrated under reduced pressure to give 6-((4-(hydroxymethyl)phenyl)amino)-5-nitropicolinamide (3.2 g, yield: 67%) as a yellow solid. MS: m/z=288.9 [M+H]⁺. ¹H NMR (400 MHz, Dimethylsulfoxide-d₆) δ 9.96 (s, 1H), 8.67 (d, J=8.4 Hz, 1H), 7.94 (s, 1H), 7.61-7.55 (m, 2H), 7.47 (d, J=8.0 Hz, 1H), 7.35 (d, J=8.4 Hz, 2H), 7.27 (s, 1H), 5.18 (t, J=5.6 Hz, 1H), 4.50 (d, J=5.6 Hz, 2H).

### Step 4: 4-((6-Carbamoyl-3-nitropyridin-2-yl)amino)benzyl acetate

To a solution of 6-((4-(hydroxymethyl)phenyl)amino)-5-nitropicolinamide (2.4 g, 8.33 mmol) in CH₂Cl₂ (20 mL) were added TEA (2.53 g, 25 mmol), Ac₂O (1.27 g, 12.5 mmol) and DMAP (101 mg, 823 μmol). The mixture was stirred at 25° C. for 1 hr. The reaction mixture was quenched with H₂O (50 mL) at 25° C., and then extracted with CH₂Cl₂ (50 mL×3). The combined organic layers were washed with brine (50 mL), dried over Na₂SO₄, filtered and concentrated under reduced pressure to give 4-((6-carbamoyl-3-nitropyridin-2-yl)amino)benzyl acetate (2.5 g, yield: 90%) as a red solid. MS: m/z=353.0 [M+Na]⁺. ¹H NMR (400 MHz, Dimethylsulfoxide-d₆) δ 9.98 (s, 1H), 8.67 (d, J=8.4 Hz, 1H), 7.94 (s, 1H), 7.69-7.62 (m, 2H), 7.49 (d, J=8.4 Hz, 1H), 7.40 (d, J=8.4 Hz, 2H), 7.36-7.29 (m, 1H), 5.07 (s, 2H), 2.07 (s, 3H).

### Step 5: 4-(2-(2-Aminopyridin-3-yl)-5-carbamoyl-3H-imidazo[4,5-b]pyridin-3-yl)benzyl acetate

To a solution of 4-((6-(dimethylcarbamoyl)-3-nitropyridin-2-yl)amino)benzyl acetate (2.2 g, 6.66 mmol) in DMSO (20 mL) were added Na₂S₂O₄ (2.32 g, 13.3 mmol) and 2-aminonicotinaldehyde (895 mg, 7.33 mmol). The mixture was stirred at 100° C. for 16 hr. The reaction mixture was quenched with H₂O (50 mL) at 25° C., and then extracted with CH₂Cl₂ (50 mL×3). The combined organic layers were washed with brine (50 mL), dried over Na₂SO₄, filtered and concentrated under reduced pressure. After purified by silica gel flash chromatography (Eluent of 50˜80% EtOAc in petroleum ether), 4-(2-(2-aminopyridin-3-yl)-5-carbamoyl-3H-imidazo[4,5-b]pyridin-3-yl)benzyl acetate (900 mg, yield: 30%) was obtained as a yellow solid. MS: m/z=403.0 [M+H]⁺.

### Step 6: 2-(2-Aminopyridin-3-yl)-3-(4-(hydroxymethyl)phenyl)-3H-imidazo[4,5-b]pyridine-5-carboxamide

To a solution of 4-(2-(2-aminopyridin-3-yl)-5-carbamoyl-3H-imidazo[4,5-b]pyridin-3-yl)benzyl acetate (900 mg, 2.24 mmol) in THF (10 mL) and MeOH (10 mL) was added K₂CO₃ (927 mg, 6.71 mmol). The mixture was stirred at 25° C. for 0.5 hr. The reaction mixture was diluted with H₂O (20 mL) and extracted with CH₂Cl₂ (20 mL×2). The combined organic layers were washed with brine (20 mL), dried over Na₂SO₄, filtered and concentrated under reduced pressure to give 2-(2-aminopyridin-3-yl)-3-(4-(hydroxymethyl)phenyl)-3H-imidazo[4,5-b]pyridine-5-carboxamide (700 mg, yield: 46%) as a yellow solid. MS: m/z=361.0 [M+H]+. ¹H NMR (400 MHz, Dimethylsulfoxide-d₆) δ 8.32 (d, J=8.4 Hz, 1H), 8.07 (d, J=8.4 Hz, 1H), 8.03-8.00 (m, 1H), 7.65-7.51 (m, 2H), 7.50-7.43 (m, 4H), 7.25 (dd, J=7.6, 1.6 Hz, 1H), 6.91 (br s, 2H), 6.44 (dd, 1=7.6, 4.8 Hz, 1H), 4.59 (s, 2H), 3.32-3.29 (m, 1H).

### Step 7: 2-(2-Aminopyridin-3-yl)-3-(4-(chloromethyl)phenyl)-3H-imidazo[4,5-b]pyridine-5-carboxamide

To a solution of 2-(2-aminopyridin-3-yl)-3-(4-(hydroxymethyl)phenyl)-3H-imidazo[4,5-b]pyridine-5-carboxamide (400 mg, 1.11 mmol) in CH₂Cl₂ (5 mL) was added SOCl₂ (396 mg, 3.33 mmol). The mixture was stirred at 40° C. for 0.5 hr. The reaction was concentrated under reduced pressure to give 2-(2-aminopyridin-3-yl)-3-(4-(chloromethyl)phenyl)-3H-imidazo[4,5-b]pyridine-5-carboxamide (Intermediate 74, 400 mg, yield: 81%) was obtained as a brown solid. MS: m/z=378.9 [M+H]⁺. ¹H NMR (400 MHz, Dimethylsulfoxide-d₆) δ 8.42 (d, J=8.4 Hz, 1H), 8.18-8.09 (m, 2H), 7.97-7.93 (m, 1H), 7.74-7.34 (m, 6H), 6.99-6.84 (m, 1H), 4.85 (s, 2H).

### Intermediate 75: 2-(2-Aminopyridin-3-yl)-3-(4-(chloromethyl)phenyl)-N,N-dimethyl-3H-imidazo[4,5-b]pyridine-5-carboxamide

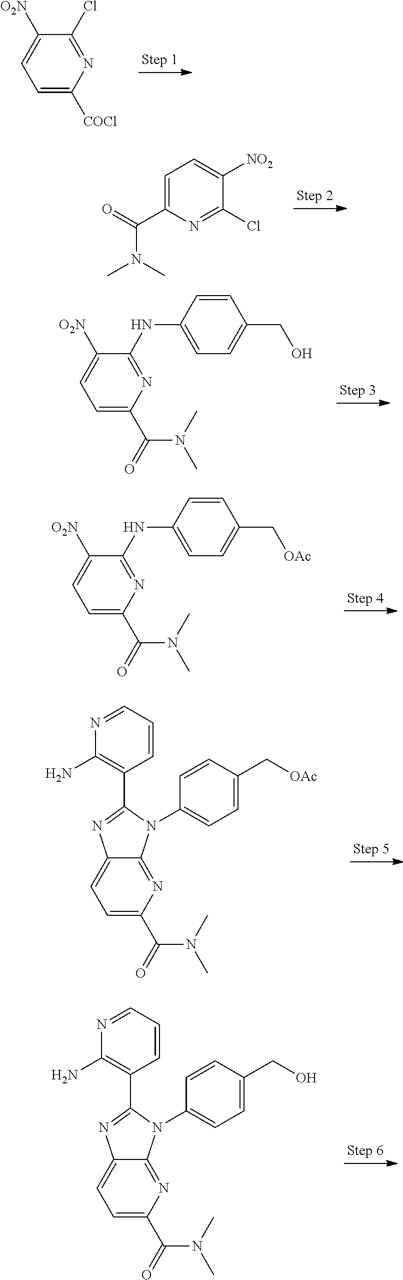

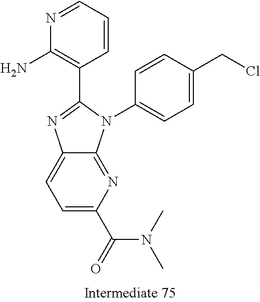 [SMILES: CN(C)C(=O)c1ccc2nc(-c3cccnc3N)n(-c3ccc(CCl)cc3)c2n1]

### Step 1: 6-Chloro-N,N-dimethyl-5-nitropicolinamide

To a solution of 6-chloro-5-nitropicolinoyl chloride (2 g, 9.05 mmol) in CH₂C12 (20 mL) were added TEA (2.75 g, 27.2 mmol) and dimethylamine (738 mg, 9.05 mmol, HCl salt). The mixture was stirred at 0° C. for 1 hr. The reaction mixture was quenched with H₂O (20 mL) at 25° C., and then diluted with CH₂Cl₂ (30 mL) and extracted with CH₂Cl₂ (50 mL×3). The combined organic layers were washed with brine (50 mL), dried over Na₂SO₄, filtered and concentrated under reduced pressure to give 6-chloro-N,N-dimethyl-5-nitropicolinamide (1.9 g, yield: 70%) as a yellow solid. MS: m/z=230.0 [M+H]+.

### Step 2: 6-((4-(Hydroxymethyl)phenyl)amino)-N,N-dimethyl-5-nitropicolinamide

To a solution of 6-chloro-N,N-dimethyl-5-nitropicolinamide (1.9 g, 8.27 mmol) in DMSO (20 mL) were added (4-aminophenyl)methanol (815 mg, 6.62 mmol) and DIEA (3.21 g, 24.8 mmol). The mixture was stirred at 110° C. for 16 hr. The reaction mixture was quenched with H₂O (20 mL) at 25° C., and extracted with CH₂Cl₂ (50 mL×3). The combined organic layers were washed with brine (50 mL), dried over Na₂SO₄, filtered and concentrated under reduced pressure to give 6-((4-(hydroxymethyl)phenyl)amino)-N,N-dimethyl-5-nitropicolinamide (2.6 g, yield: 79%) as a red solid. MS: m/z=317.0 [M+H]⁺.

### Step 3: 4-((6-(Dimethylcarbamoyl)-3-nitropyridin-2-yl)amino)benzyl acetate

To a solution of 6-((4-(hydroxymethyl)phenyl)amino)-N,N-dimethyl-5-nitropicolinamide (2.6 g, 8.22 mmol) in CH₂Cl₂ (20 mL) were added TEA (2.50 g, 24.7 mmol), Ac₂O (1.26 g, 12.3 mmol) and DMAP (100 mg, 822 μmol). The mixture was stirred at 25° C. for 1 hr. The reaction mixture was quenched with H₂O (50 mL) at 25° C., and extracted with CH₂Cl₂ (50 mL×3). The combined organic layers were washed with brine (50 mL), dried over Na₂SO₄, filtered and concentrated under reduced pressure. After purified by silica gel flash chromatography (Eluent of 1˜30% EtOAc in petroleum ether), 4-((6-(dimethylcarbamoyl)-3-nitropyridin-2-yl)amino)benzyl acetate (1.7 g, yield: 55%) was obtained as a red solid. MS: m/z=380.9 [M+Na]⁺.

### Step 4: 4-(2-(2-Aminopyridin-3-yl)-5-(dimethylcarbamoyl)-3H-imidazo[4,5-b]pyridin-3-yl)benzyl acetate

To a solution of 4-((6-(dimethylcarbamoyl)-3-nitropyridin-2-yl)amino)benzyl acetate (1.6 g, 4.46 mmol) in DMSO (20 mL) were added Na₂S₂O₄ (3.57 g, 17.9 mmol) and 2-aminonicotinaldehyde (654 mg, 5.36 mmol). The mixture was stirred at 100° C. for 16 hr. The reaction mixture was quenched with H₂O (25 mL) at 25° C. and extracted with CH₂Cl₂ (50 mL×3). The combined organic layers were washed with brine (50 mL), dried over Na₂SO₄, filtered and concentrated under reduced pressure. After purified by silica gel flash chromatography (Eluent of 50%˜100% EtOAc in petroleum ether), 4-(2-(2-aminopyridin-3-yl)-5-(dimethylcarbamoyl)-3H-imidazo[4,5-b]pyridin-3-yl)benzyl acetate (370 mg, yield: 19%) was obtained as a brown solid. MS: m/z=431.1 [M+H]⁺.

### Step 5: 2-(2-Aminopyridin-3-yl)-3-(4-(hydroxymethyl)phenyl)-N,N-dimethyl-3H-imidazo[4,5-b]pyridine-5-carboxamide

To a solution of 4-(2-(2-aminopyridin-3-yl)-5-(dimethylcarbamoyl)-3H-imidazo[4,5-b]pyridin-3-yl)benzyl acetate (370 mg, 860 μmol) in THF (5 mL) and MeOH (5 mL) was added K₂C03 (356 mg, 2.58 mmol). The mixture was stirred at 25° C. for 0.5 hr. The reaction mixture was diluted with H₂O (10 mL) and extracted with CH₂Cl₂ (15 mL×2). The combined organic layers were washed with brine (10 mL), dried over Na₂SO₄, filtered and concentrated under reduced pressure to give 2-(2-aminopyridin-3-yl)-3-(4-(hydroxymethyl)phenyl)-N,N-dimethyl-3H-imidazo[4,5-b]pyridine-5-carboxamide (320 mg, yield: 74%) as a yellow solid. MS: m/z=389.1 [M+H]⁺.

### Step 6: 2-(2-Aminopyridin-3-yl)-3-(4-(chloromethyl)phenyl)-NJN-dimethyl-3H-imidazo[4,5-b]pyridine-5-carboxamide

To a solution of 2-(2-aminopyridin-3-yl)-3-(4-(hydroxymethyl)phenyl)-N,N-dimethyl-3H-imidazo[4,5-b]pyridine-5-carboxamide (320 mg, 824 μmol) in CH₂Cl₂ (5 mL) was added SOCl₂ (294 mg, 2.47 mmol). The mixture was stirred at 40° C. for 0.4 hr. The reaction was concentrated under reduced pressure to give 2-(2-aminopyridin-3-yl)-3-(4-(chloromethyl)phenyl)-N,N-dimethyl-3H-imidazo[4,5-b]pyridine-5-carboxamide (Intermediate 75, 330 mg, yield: 91%) as a yellow solid. MS: m/z=407.0 [M+H]⁺.

### Intermediate 76: 3-(3-(4-(Chloromethyl)phenyl)-5-(2-chlorophenyl)-3H-imidazo[4,5-b]pyridin-2-yl)pyridin-2-amine

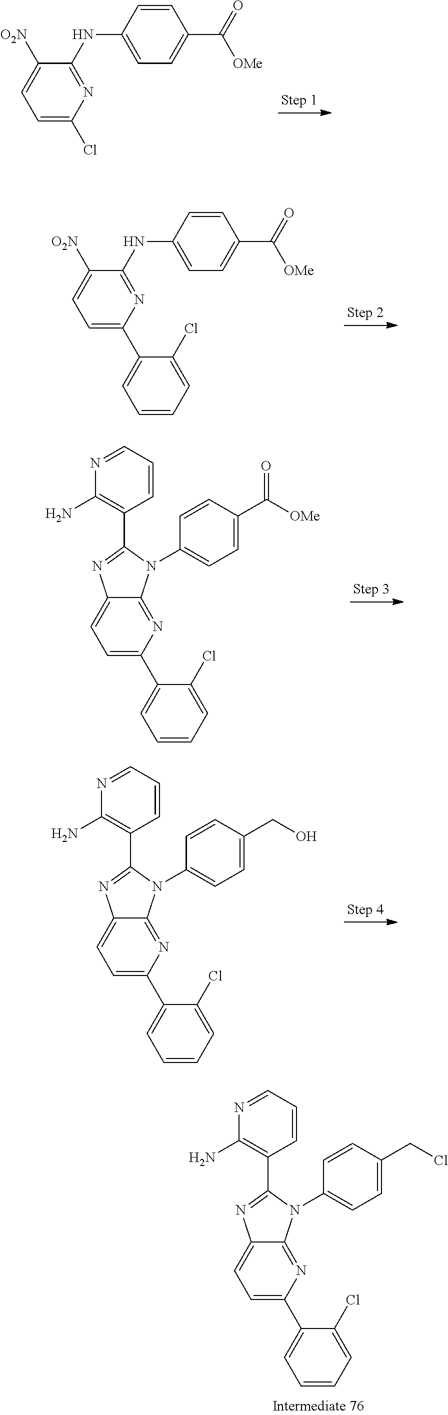

### Step 1: Methyl 4-((6-(2-chlorophenyl)-3-nitropyridin-2-yl)amino)benzoate

To a mixture of methyl 4-((6-chloro-3-nitropyridin-2-yl)amino)benzoate (refer to Intermediate 13 for detail procedures, 5 g, 16.2 mmol), (2-chlorophenyl)boronic acid (3.3 g, 21.1 mmol), Pd(dppf)Cl₂ (1.19 g, 1.63 mmol), Cs₂CO₃ (15.8 g, 48.7 mmol) in dioxane (50 mL) and H₂O (5 mL) was degassed and purged with N₂ three times, and the mixture was stirred at 100° C. for 12 hr under N₂ atmosphere. The reaction mixture was diluted with water (20 mL) and extracted with CH₂Cl₂ (30 mL×2). The combined organic layers were dried over Na₂SO₄, filtered and concentrated under reduced pressure. The residue was purified by silica gel flash chromatography (Eluent of 0˜10% MeOH in CH₂Cl₂) to give methyl 4-((6-(2-chlorophenyl)-3-nitropyridin-2-yl)amino)benzoate (1.5 g, yield: 21%) as a yellow solid. MS: m/z=384.0 [M+H]⁺. ¹H NMR (400 MHz, Chloroform-d) δ 10.49 (br s, 1H), 8.63 (d, J=8.8 Hz, 1H), 8.04 (d, 1=6.8 Hz, 2H), 7.90 (d, J=9.2 Hz, 2H), 7.68-7.63 (m, 1H), 7.56-7.52 (m, 1H), 7.46-7.39 (m, 2H), 7.33 (d, J=8.8 Hz, 1H), 3.91 (s, 3H).

### Step 2: Methyl 4-(2-(2-aminopyridin-3-yl)-5-(2-chlorophenyl)-3H-imidazo[4,5-b]pyridin-3-yl)benzoate

To a solution of methyl 4-((6-(2-chlorophenyl)-3-nitropyridin-2-yl)amino)benzoate (1.5 g, 3.91 mmol) and 2-aminonicotinaldehyde (525 mg, 4.30 mmol) in DMSO (45 mL) was added Na₂S₂O₄ (3.20 g, 15.6 mmol). The mixture was stirred at 100° C. for 12 hr. The reaction mixture was diluted with water (100 mL) and extracted with CH₂Cl₂ (300 mL×2). The combined organic layers were washed with brine (100 mL), dried over Na₂SO₄, filtered and concentrated under reduced pressure. The residue was purified by silica gel flash chromatography (Eluent of 0˜100% EtOAc in petroleum ether) to give methyl 4-(2-(2-aminopyridin-3-yl)-5-(2-chlorophenyl)-3H-imidazo[4, 5-b]pyridin-3-yl) benzoate (800 mg, yield: 41%) as a yellow solid. MS: m/z=456.0 [M+H]⁺. ¹H NMR (400 MHz, Chloroform-d) δ 8.23-8.15 (m, 3H), 8.07 (dd, J=4.8, 1.6 Hz, 1H), 7.73 (d, J=8.4 Hz, 1H), 7.60-7.52 (m, 3H), 7.50-7.46 (m, 1H), 7.36-7.31 (m, 2H), 7.10 (dd, J=7.6, 1.6 Hz, 1H), 6.92 (br s, 2H), 6.43 (dd, J=8.0, 5.2 Hz, 1H), 3.96 (s, 3H).

### Step 3: (4-(2-(2-Aminopyridin-3-yl)-5-(2-chlorophenyl)-3H-imidazo[4,5-b]pyridin-3-yl)phenyl)methanol

To a solution of methyl 4-(2-(2-aminopyridin-3-yl)-5-(2-chlorophenyl)-3H-imidazo[4,5-b]pyridin-3-yl)benzoate (800 mg, 1.75 mmol) in THF (50 mL) was added LiAlH₄ (2.5 M, 1.05 mL) at 0° C. The mixture was stirred at 25° C. for 2 hr. The reaction mixture was quenched with Na₂SO₄·10H₂O (120 mg) at 0° C. and filtered. The filtrate was concentrated under reduced pressure to give (4-(2-(2-aminopyridin-3-yl)-5-(2-chlorophenyl)-3H-imidazo[4,5-b]pyridin-3-yl)phenyl)methanol (700 mg, crude as a yellow solid.

### Step 4: 3-(3-(4-(Chloromethyl)phenyl)-5-(2-chlorophenyl)-3H-imidazo[4,5-b]pyridin-2-yl)pyridin-2-amine

To a solution of (4-(2-(2-aminopyridin-3-yl)-5-(2-chlorophenyl)-3H-imidazo[4,5-b]pyridin-3-yl)phenyl)methanol (700 mg, 1.64 mmol) in CH₂Cl₂ (10 mL) was added SOCl₂ (1.17 g, 9.82 mmol). The mixture was stirred at 40° C. for 2 hr. The mixture was concentrated under reduced pressure. 3-(3-(4-(Chloromethyl)phenyl)-5-(2-chlorophenyl)-3H-imidazo[4,5-b]pyridin-2-yl)pyridin-2-amine (Intermediate 76, 750 mg HCl salt) was obtained as a yellow solid. MS: m/z=446.0 [M+H]⁺.

### Intermediate 77: 3-(5-(2-Chlorophenyl)-3-(4-(piperazin-1-ylmethyl)phenyl)-3H-imidazo[4,5-b]pyridin-2-yl)pyridin-2-amine

### Step 1: tert-Butyl 4-(4-(2-(2-aminopyridin-3-yl)-5-(2-chlorophenyl)-3H-imidazo[4,5-b]pyridin-3-yl)benzyl)piperazine-1-carboxylate

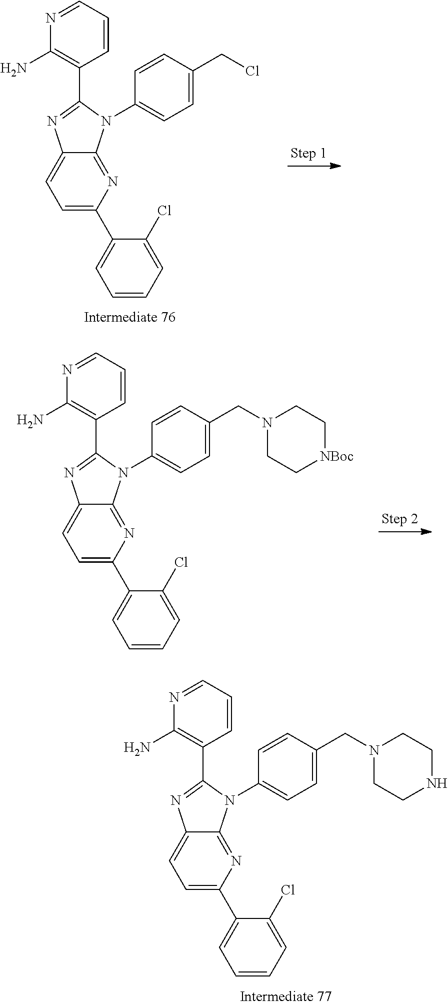 [SMILES: Nc1ncccc1-c1nc2ccc(-c3ccccc3Cl)nc2n1-c1ccc(CCl)cc1 ; CC(C)(C)OC(=O)N1CCN(Cc2ccc(-n3c(-c4cccnc4N)nc4ccc(-c5ccccc5Cl)nc43)cc2)CC1 ; Nc1ncccc1-c1nc2ccc(-c3ccccc3Cl)nc2n1-c1ccc(CN2CCNCC2)cc1]

To a solution of Intermediate 76 (1 g, 2.24 mmol) and tert-butyl piperazine-1-carboxylate (417 mg, 2.24 mmol) in MeCN (10 mL) were added NaI (67.2 mg, 448 μmol) and K₂CO₃ (929 mg, 6.72 mmol). The mixture was stirred at 25° C. for 16 hr. The residue was diluted with H₂O (30 mL) and extracted with EtOAc (30 mL×3). The combined organic layers were washed with brine (20 mL×2), dried over Na₂SO₄, filtered and concentrated under reduced pressure. The residue was purified by silica gel flash chromatography (Eluent of 0˜3% MeOH in CH₂Cl₂) to give tert-butyl 4-(4-(2-(2-aminopyridin-3-yl)-5-(2-chlorophenyl)-3H-imidazo[4,5-b]pyridin-3-yl)benzyl)piperazine-1-carboxylate (600 mg, yield: 45%) as a yellow solid. MS: m/z=596.1 [M+H]⁺.

### Step 6: 3-(5-(2-Chlorophenyl)-3-(4-(piperazin-1-ylmethyl)phenyl)-3H-imidazo[4,5-b]pyridin-2-yl)pyridin-2-amine

To a solution of tert-butyl 4-(4-(2-(2-aminopyridin-3-yl)-5-(2-chlorophenyl)-3H-imidazo[4,5-b]pyridin-3-yl)benzyl)piperazine-1-carboxylate (600 mg, 1.01 mmol) in MeOH (2 mL) was added HCl in 1,4-dioxane (4 M, 5 mL). The mixture was stirred at 25° C. for 3 hr. The reaction mixture was filtered and concentrated under reduced pressure to give 3-(5-(2-chlorophenyl)-3-(4-(piperazin-1-ylmethyl)phenyl)-3H-imidazo[4,5-b]pyridin-2-yl)pyridin-2-amine (Intermediate 77, HCl salt, 530 mg, yield: 90%). MS: m/z=496.3 [M+H]+. ¹H NMR (400 MHz, Methanol-d₄) δ 8.23 (d, J=8.4 Hz, 1H), 8.01-7.96 (m, 1H), 7.66 (d, J=8.0 Hz, 1H), 7.56-7.48 (m, 4H), 7.44-7.32 (m, 5H), 6.48 (dd, J=7.6, 5.6 Hz, 1H), 3.58 (s, 2H), 2.87-2.84 (m, 4H), 2.52-2.44 (m, 4H).

### Intermediate 78: 4-(2-(2-Aminopyridin-3-yl)-3-(4-(piperazin-1-ylmethyl)phenyl)-3H-imidazo[4,5-b]pyridin-5-yl)benzonitrile

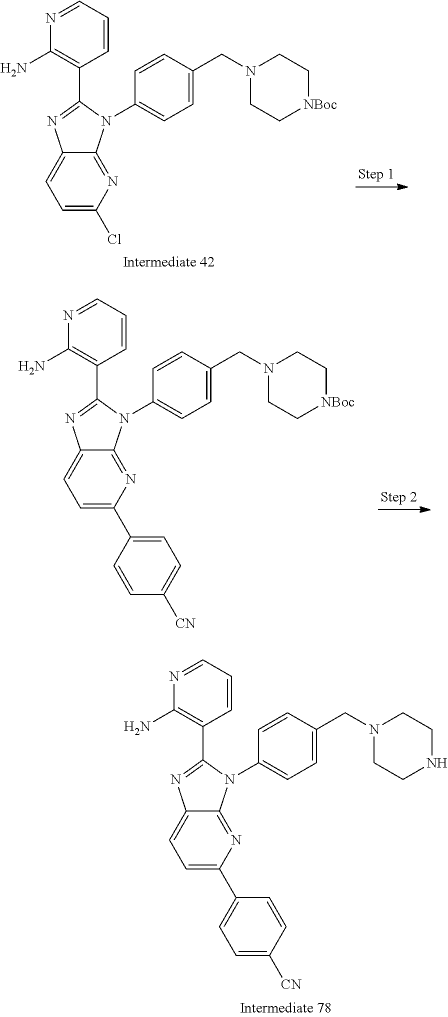

### Step 1: Tert-butyl 4-(4-(2-(2-aminopyridin-3-yl)-5-(4-cyanophenyl)-3H-imidazo[4,5-b]pyridin-3-yl)benzyl)piperazine-1-carboxylate

A mixture of Intermediate 42 (220 mg, 423 μmol), (4-cyanophenyl)boronic acid (68.4 mg, 465 μmol), Cs₂CO₃ (414 mg, 1.27 mmol), Pd(dppf)Cl₂ (155 mg, 212 μmol) in 1,4-dioxane (5 mL) and H₂O (1 mL) was degassed and purged with N₂ three times, and then the mixture was stirred at 100° C. for 16 hr under N₂ atmosphere. The reaction mixture was filtered at 25° C., and then diluted with H₂O (20 mL) and extracted with CH₂Cl₂ (20 mL×3). The combined organic layers were washed with brine (30 mL), dried over Na₂SO₄, filtered and concentrated under reduced pressure. After purified by silica gel flash chromatography (Eluent of 0˜30% EtOAc in petroleum ether), tert-butyl 4-(4-(2-(2-aminopyridin-3-yl)-5-(3-cyanophenyl)-3H-imidazo[4,5-b]pyridin-3-yl)benzyl)piperazine-1-carboxylate (220 mg, yield: 89%) was obtained as a brown solid. MS: m/z=587.4 [M+H]⁺.

### Step 2: 4-(2-(2-Aminopyridin-3-yl)-3-(4-(piperazin-1-ylmethyl)phenyl)-3H-imidazo[4,5-b]pyridin-5-yl)benzonitrile

To a solution of tert-butyl 4-(4-(2-(2-aminopyridin-3-yl)-5-(3-cyanophenyl)-3H-imidazo[4,5-b]pyridin-3-yl)benzyl)piperazine-1-carboxylate (220 mg, 375 μmol) in 1,4-dioxane (3 mL) was added 4 M HCl in 1,4-dioxane (3 mL) at 25° C. The mixture was stirred at 25° C. for 16 hr. The reaction mixture was then filtered. The collected solid was washed with 1,4-dioxane (2 mL×2) and dried in vacuo to give 4-(2-(2-aminopyridin-3-yl)-3-(4-(piperazin-1-ylmethyl)phenyl)-3H-imidazo[4,5-b]pyridin-5-yl)benzonitrile (Intermediate 78, 192 mg HCl solid, yield: 98%) as a yellow solid. MS: m/z=487.2 [M+H]⁺. ¹H NMR (400 MHz, Methanol-d₄) δ 8.38 (d, J=8.4 Hz, 1H), 8.25 (d, J=8.4 Hz, 2H), 8.13 (d, J=8.4 Hz, 1H), 8.04 (d, J=5.6 Hz, 1H), 7.95 (d, J=7.6 Hz, 2H), 7.89 (d, J=7.2 Hz, 1H), 7.81 (d, 1=8.4 Hz, 2H), 7.74 (d, J=7.6 Hz, 2H), 6.93 (t, J=6.8 Hz, 1H), 3.71-3.70 (m, 6H), 3.68-3.64 (m, 4H).

### Intermediate 79: 3-(5-(3-Fluorophenyl)-3-(4-(piperazin-1-ylmethyl)phenyl)-3H-imidazo[4,5-b]pyridin-2-yl)pyridin-2-amine

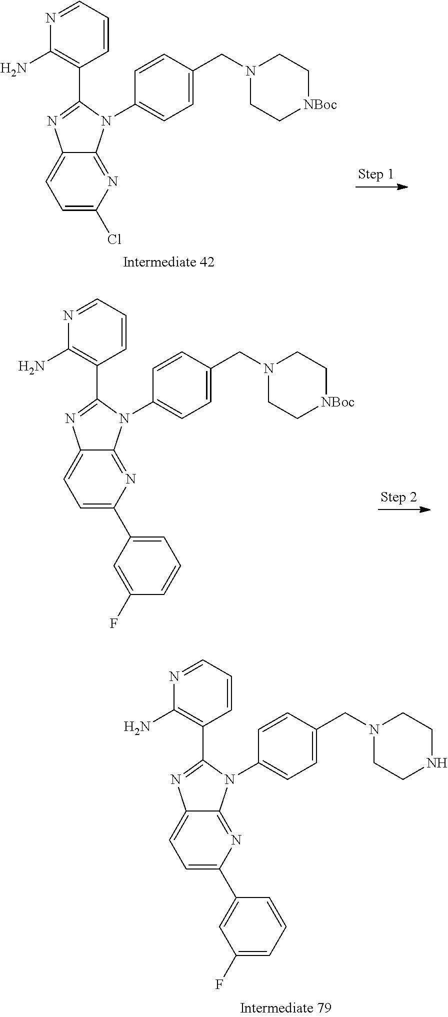 [SMILES: CC(C)(C)OC(=O)N1CCN(Cc2ccc(-n3c(-c4cccnc4N)nc4ccc(Cl)nc43)cc2)CC1 ; CC(C)(C)OC(=O)N1CCN(Cc2ccc(-n3c(-c4cccnc4N)nc4ccc(-c5cccc(F)c5)nc43)cc2)CC1 ; Nc1ncccc1-c1nc2ccc(-c3cccc(F)c3)nc2n1-c1ccc(CN2CCNCC2)cc1]

### Step 1: Tert-butyl 4-(4-(2-(2-aminopyridin-3-yl)-5-(3-fluorophenyl)-3H-imidazo[4,5-b]pyridin-3-yl)benzyl)piperazine-1-carboxylate

A mixture of Intermediate 42 (200 mg, 385 μmol), (3-fluorophenyl)boronic acid (59.2 mg, 423 μmol), CS₂CO₃ (376 mg, 1.15 mmol), Pd(dppf)Cl₂ (141 mg, 192 μmol) in 1,4-dioxane (5 mL) and H₂O (1 mL) was degassed and purged with N₂ three times, and then the mixture was stirred at 100° C. for 16 hr under N₂ atmosphere. The reaction mixture was filtered at 25° C., and then diluted with H₂O (20 mL) and extracted with CH₂C12 (20 mL×3). The combined organic layers were washed with brine (30 mL), dried over Na₂SO₄, filtered and concentrated under reduced pressure. After purified by silica gel flash chromatography (Eluent of 0˜30% EtOAc in petroleum ether), tert-butyl 4-(4-(2-(2-aminopyridin-3-yl)-5-(3-fluorophenyl)-3H-imidazo[4,5-b]pyridin-3-yl)benzyl)piperazine-1-carboxylate (250 mg, yield: 98%) was obtained as a yellow solid. MS: m/z=580.2 [M+H]⁺.

### Step 2: 3-(5-(3-Fluorophenyl)-3-(4-(piperazin-1-ylmethyl)phenyl)-3H-imidazo[4,5-b]pyridin-2-yl)pyridin-2-amine

To a solution of tert-butyl 4-(4-(2-(2-aminopyridin-3-yl)-5-(3-fluorophenyl)-3H-imidazo[4,5-b]pyridin-3-yl)benzyl)piperazine-1-carboxylate (200 mg, 345 μmol) in 1,4-dioxane (3 mL) was added 4 M HCl in 1,4-dioxane (3 mL) at 25° C. The mixture was stirred at 25° C. for 16 hr. The reaction mixture was then filtered and the collected solid was washed with 1,4-dioxane (2 mL×2) and dried in vacuo to give 3-(5-(3-fluorophenyl)-3-(4-(piperazin-1-ylmethyl)phenyl)-3H-imidazo[4,5-b]pyridin-2-yl)pyridin-2-amine (Intermediate 79, 174 mg HCl salt, yield: 98%) as a yellow solid. MS: m/z=480.1 [M+H]⁺. ¹H NMR (400 MHz, Methanol-d₄) δ 8.34 (d, J=8.4 Hz, 1H), 8.08-8.01 (m, 2H), 7.98-7.86 (m, 4H), 7.81-7.71 (m, 3H), 7.52-7.42 (m, 1H), 7.18-7.12 (m, 1H), 6.98-6.85 (m, 1H), 3.69-3.67 (m, 4H), 3.35 (s, 2H), 3.34-3.31 (m, 4H). ¹⁹F NMR (400 MHz, Methanol-d₄) δ −115.02.

### Intermediate 80: 3-(5-(3-Chlorophenyl)-3-(4-(piperazin-1-ylmethyl)phenyl)-3H-imidazo[4,5-b]pyridin-2-yl)pyridin-2-amine

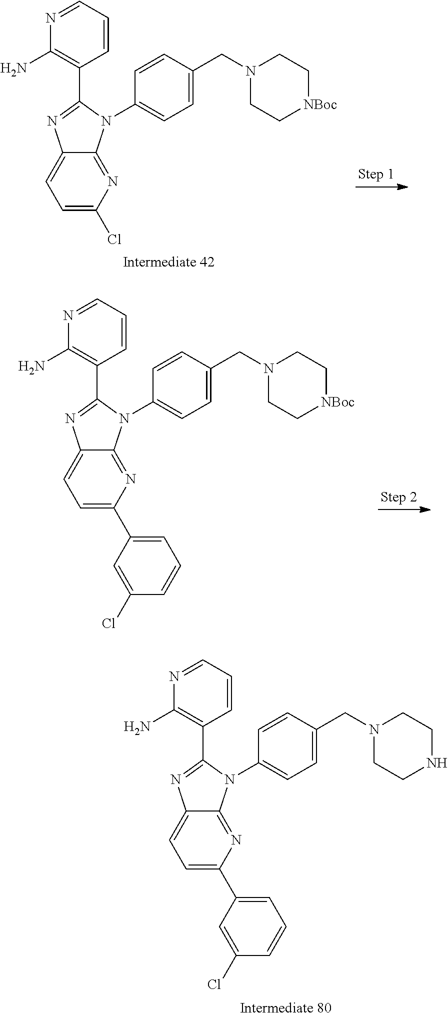 [SMILES: CC(C)(C)OC(=O)N1CCN(Cc2ccc(-n3c(-c4cccnc4N)nc4ccc(Cl)nc43)cc2)CC1 ; CC(C)(C)OC(=O)N1CCN(Cc2ccc(-n3c(-c4cccnc4N)nc4ccc(-c5cccc(Cl)c5)nc43)cc2)CC1 ; Nc1ncccc1-c1nc2ccc(-c3cccc(Cl)c3)nc2n1-c1ccc(CN2CCNCC2)cc1]

### Step 1: tert-Butyl 4-(4-(2-(2-aminopyridin-3-yl)-5-(3-chlorophenyl)-3H-imidazo[4,5-b]pyridin-3-yl)benzyl)piperazine-1-carboxylate

A mixture of Intermediate 42 (200 mg, 385 μmol), (3-chlorophenyl)boronic acid (66.2 mg, 423 μmol), Cs₂CO₃ (376 mg, 1.15 mmol), Pd(dppf)Cl₂ (141 mg, 192 μmol) in 1,4-dioxane (5 mL) and H₂O (1 mL) was degassed and purged with N₂ three times, and then the mixture was stirred at 100° C. for 16 hr under N₂ atmosphere. The reaction mixture was filtered at 25° C., and then diluted with H₂O (20 mL) and extracted with CH₂Cl₂ (20 mL×3). The combined organic layers were washed with brine (30 mL), dried over Na₂SO₄, filtered and concentrated under reduced pressure. After purified by silica gel flash chromatography (Eluent of 0˜30% EtOAc in petroleum ether), tert-butyl 4-(4-(2-(2-aminopyridin-3-yl)-5-(3-chlorophenyl)-3H-imidazo[4,5-b]pyridin-3-yl)benzyl)piperazine-1-carboxylate (200 mg, yield: 77%) was obtained as a yellow solid. MS: m/z=596.3 [M+H]⁺.

### Step 2: 3-(5-(3-Chlorophenyl)-3-(4-(piperazin-1-ylmethyl)phenyl)-3H-imidazo[4,5-h]pyridin-2-yl)pyridin-2-amine

To a solution of tert-butyl 4-(4-(2-(2-aminopyridin-3-yl)-5-(3-chlorophenyl)-3H-imidazo[4,5-b]pyridin-3-yl)benzyl)piperazine-1-carboxylate (150 mg, 252 μmol) in 1,4-dioxane (3 mL) was added 4 M HCl in 1,4-dioxane (3 mL) at 25° C. The mixture was stirred at 25° C. for 16 hr. The reaction mixture was then filtered and the collected solid was washed with 1,4-dioxane (2 mL×2) and dried in vacuo to give 3-(5-(3-chlorophenyl)-3-(4-(piperazin-1-ylmethyl)phenyl)-3H-imidazo[4,5-h]pyridin-2-yl)pyridin-2-amine (Intermediate 80, 113 mg, HCl salt, yield: 84%) as a yellow solid. MS: m/z=496.1 [M+H]⁺. ¹H NMR (400 MHz, Methanol-d₄) δ 8.21 (d, J=8.4 Hz, 1H), 8.04 (s, 1H), 8.01-7.92 (m, 3H), 7.55 (d, J=8.0 Hz, 2H), 7.45 (d, J=8.4 Hz, 2H), 7.43-7.33 (m, 3H), 6.48 (dd, J=7.6, 5.2 Hz, 1H), 3.64 (s, 2H), 2.92 (t, J=5.2 Hz, 4H), 2.50-2.58 (m, 4H).

### Intermediate 81: 3-(3-(4-((4-Aminopiperidin-1-yl)methyl)phenyl)-5-cyclopropyl-3H-imidazo[4,5-b]pyridin-2-yl)pyridin-2-amine

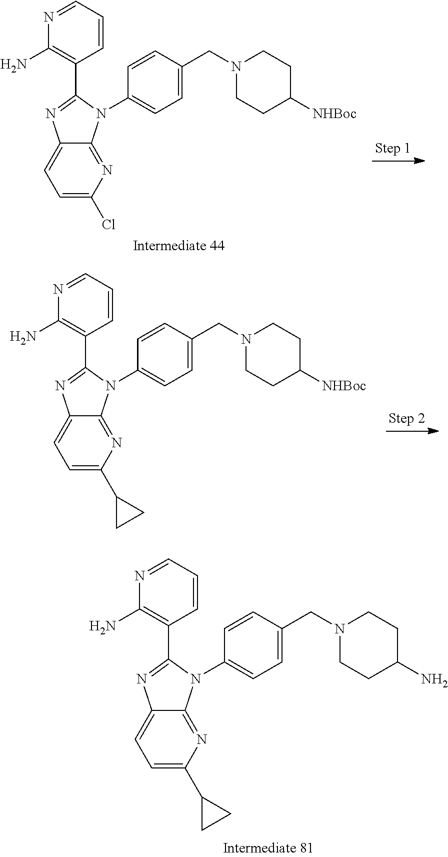 [SMILES: CC(C)(C)OC(=O)NC1CCN(Cc2ccc(-n3c(-c4cccnc4N)nc4ccc(Cl)nc43)cc2)CC1 ; CC(C)(C)OC(=O)NC1CCN(Cc2ccc(-n3c(-c4cccnc4N)nc4ccc(C5CC5)nc43)cc2)CC1 ; Nc1ncccc1-c1nc2ccc(C3CC3)nc2n1-c1ccc(CN2CCC(N)CC2)cc1]

### Step 1: tert-Butyl (1-(4-(2-(2-aminopyridin-3-yl)-5-cyclopropyl-3H-imidazo[4,5-b]pyridin-3-yl)benzyl)piperidin-4-yl)carbamate

A mixture of Intermediate 44 (300 mg, 562 μmol), cyclopropylboronic acid (97 mg, 1.1 mmol), t-BuOK (126 mg, 1.1 mmol), cataCXiumAPdG3 (41 mg, 56 μmol) in H₂O (2 mL) and 1,4-dioxane (8 mL) was degassed and purged with N₂ three times, and then the mixture was stirred at 100° C. for 16 hr under N₂ atmosphere. The reaction mixture was quenched with H₂O (30 mL) at 25° C., and extracted with EtOAc (30 mL×2). The combined organic layers were washed with brine (30 mL), dried over Na₂SO₄, filtered, and concentrated under reduced pressure. The crude was purified by silica gel flash chromatography (Eluent of 0˜100% EtOAc in petroleum ether) to give tert-butyl (1-(4-(2-(2-aminopyridin-3-yl)-5-cyclopropyl-3H-imidazo[4,5-b]pyridin-3-yl)benzyl)piperidin-4-yl)carbamate (120 mg, yield: 40%) as a yellow solid. MS: m/z=540.3 [M+H]⁺. ¹H NMR (400 MHz, Chloroform-d) δ 8.03 (dd, J=4.8, 1.6 Hz, 1H), 7.92 (d, J=8.4 Hz, 1H), 7.48-7.43 (m, 2H), 7.31-7.28 (m, 2H), 7.12 (d, J=8.4 Hz, 1H), 7.02 (dd, J=7.6, 1.6 Hz, 1H), 6.52 (br s, 2H), 6.33 (dd, J=7.6, 4.8 Hz, 1H), 4.52-4.41 (m, 1H), 3.68-3.51 (m, 3H), 2.98-2.85 (m, 2H), 2.30-2.17 (m, 2H), 2.12-2.11 (m, 1H), 2.01-1.95 (m, 2H), 1.45-1.43 (m, 11H), 0.96-0.92 (m, 4H).

### Step 2: 3-(3-(4-((4-Aminopiperidin-1-yl)methyl)phenyl)-5-cyclopropyl-3H-imidazo[4,5-b]pyridin-2-yl)pyridin-2-amine

To a solution of tert-butyl (1-(4-(2-(2-aminopyridin-3-yl)-5-cyclopropyl-3H-imidazo[4,5-b]pyridin-3-yl)benzyl)piperidin-4-yl)carbamate (130 mg, 241 μmol) in HCl in 1,4-dioxane (4 M, 3 mL). The mixture was stirred at 25° C. for 1 hr. Then the mixture was concentrated under reduced pressure to give 3-(3-(4-((4-aminopiperidin-1-yl)methyl)phenyl)-5-cyclopropyl-3H-imidazo[4,5-b]pyridin-2-yl)pyridin-2-amine (Intermediate 81, 110 mg HCl salt, yield: 96%) was obtained as a light yellow solid. MS: m/z=440.1 [M+H]⁺. ¹H NMR (400 MHz, Methanol-d₄) δ 8.16-8.06 (m, 1H), 8.05-7.95 (m, 1H), 7.98-7.85 (m, 3H), 7.61 (d, J=7.2 Hz, 2H), 7.41-7.30 (m, 1H), 6.96-6.79 (m, 1H), 4.48 (s, 2H), 3.68 (d, J=11.6 Hz, 2H), 3.60-3.65 (m, 2H), 3.29-3.23 (m, 1H), 2.31 (d, J=12.8 Hz, 2H), 2.26-2.03 (m, 3H), 1.07-0.88 (m, 4H).

### Intermediate 82: 3-(3-(4-(Chloromethyl)phenyl)-5-(tetrahydro-2H-pyran-4-yl)-3H-imidazo[4,5-b]pyridin-2-yl)pyridin-2-amine

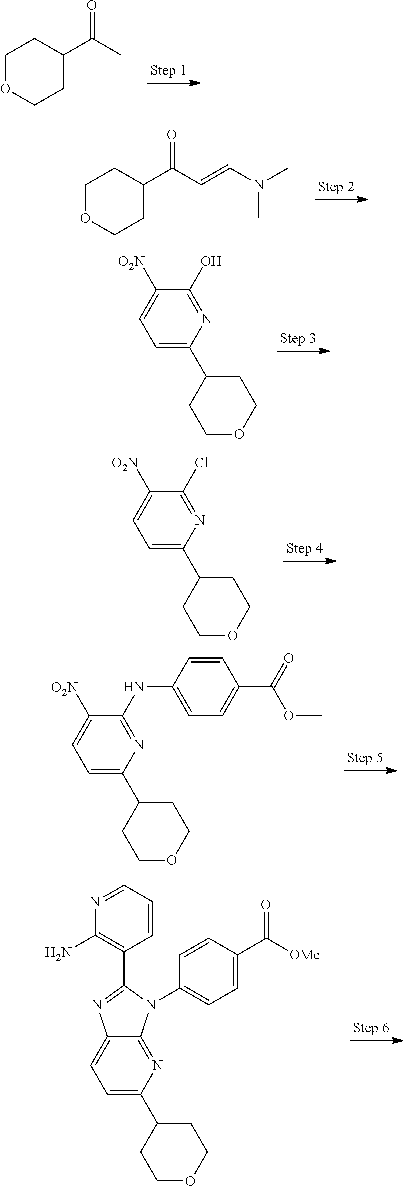

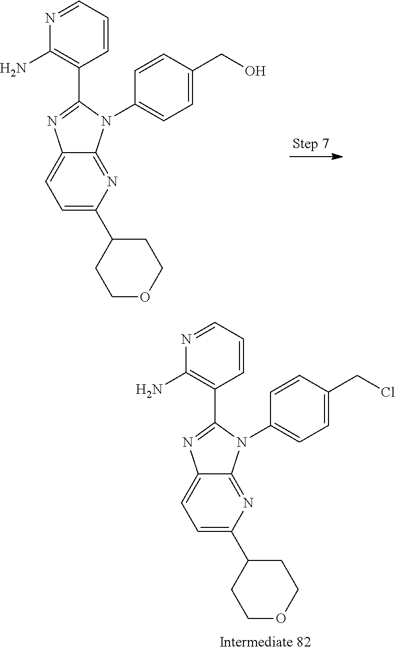 [SMILES: Nc1ncccc1-c1nc2ccc(C3CCOCC3)nc2n1-c1ccc(CO)cc1 ; Nc1ncccc1-c1nc2ccc(C3CCOCC3)nc2n1-c1ccc(CCl)cc1]

### Step 1: (E)-3-(dimethylamino)-1-(tetrahydro-2H-pyran-4-yl)prop-2-en-1-one

To a solution of 1-(tetrahydro-2H-pyran-4-yl)ethan-1-one (10 g, 78 mmol) in DMF (100 mL) was added DMF-DMA (18.6 g, 156 mmol) and CH₃ONa (422 mg, 7.8 mmol). The mixture was stirred at 100° C. for 16 hr. The reaction mixture was quenched with H₂O (500 mL) at 25° C., and extracted with EtOAc (500 mL×2). The combined organic layers were washed with brine (500 mL), dried over Na₂SO₄, filtered and concentrated under reduced pressure to give a (E)-3-(dimethylamino)-1-(tetrahydro-2H-pyran-4-yl)prop-2-en-1-one (12.56 g, crude) as light yellow oil, which was used to the next step directly. MS: m/z=184.0 [M+H]⁺.

### Step 2: 3-Nitro-6-(tetrahydro-2H-pyran-4-yl) pyridin-2-ol

To a solution of (E)-3-(dimethylamino)-1-(tetrahydro-2H-pyran-4-yl)prop-2-en-1-one (2.3 g, 13 mmol) in H₂O (20 mL) were added piperidine acetic acid salt (911 mg, 6.3 mmol) and 2-nitroacetamide (2.0 g, 18.8 mmol). The mixture was stirred at 25° C. for 16 hr. The reaction mixture was filtered, and the filter cake was washed with H₂O (20 mL), dried in reduced pressure to give a 3-nitro-6-(tetrahydro-2H-pyran-4-yl) pyridin-2-ol (1.2 g, yield: 41%) as a light yellow solid. MS: m/z=225.0 [M+H]⁺. ¹H NMR (400 MHz, Dimethylsulfoxide-d₆) δ 12.78 (br s, 1H), 8.41 (d, J=8.0 Hz, 1H), 6.30 (d, J=8.0 Hz, 1H), 3.97-3.89 (m, 2H), 3.39-3.32 (m, 2H), 2.89-2.79 (m, 1H), 1.77-1.61 (m, 4H).

### Step 3: 2-Chloro-3-nitro-6-(tetrahydro-2H-pyran-4-yl)pyridine

A solution of 3-nitro-6-(tetrahydro-2H-pyran-4-yl) pyridin-2-ol (4.5 g, 20 mmol) in POCl₃ (40 mL) was stirred at 80° C. for 16 hr. The reaction mixture was quenched with H₂O (80 mL) at 25° C., and then diluted with Na₂CO₃ aqueous solution (80 mL) and extracted with EtOAc (100 mL×2). The combined organic layers were washed with brine (100 mL), dried over Na₂SO₄, filtered and concentrated under reduced pressure to give a 2-chloro-3-nitro-6-(tetrahydro-2H-pyran-4-yl)pyridine (4.1 g, crude) as a brown oil, which was used to the next step directly. MS: m/z=242.9 [M+H]⁺.

### Step 4: Methyl 4-((3-nitro-6-(tetrahydro-2H-pyran-4-yl)pyridin-2-yl)amino)benzoate

To a solution of 2-chloro-3-nitro-6-(tetrahydro-2H-pyran-4-yl)pyridine (1.1 g, 4.5 mmol) in DIEA (15 mL) was added methyl 4-aminobenzoate (754 mg, 4.9 mmol). The mixture was stirred at 130° C. for 16 hr. The reaction mixture was poured into H₂O (100 mL), extracted with EtOAc (100 mL×2). The organic phase was dried over anhydrous Na₂SO₄, filtered and concentrated under reduced pressure. The crude product was purified by silica gel flash chromatography (Eluent of 0˜18% EtOAc in petroleum ether) to give methyl 4-((3-nitro-6-(tetrahydro-2H-pyran-4-yl) pyridin-2-yl) amino) benzoate (500 mg, yield: 31%) as a yellow solid. MS: m/z=358.0 [M+H]⁺. ¹H NMR (400 MHz, Dimethylsulfoxide-d₆) δ 10.17 (s, 1H), 8.50 (d, J=8.4 Hz, 1H), 7.95 (d, J=8.8 Hz, 2H), 7.89 (d, J=8.8 Hz, 2H), 7.02 (d, J=8.4 Hz, 1H), 4.01-3.93 (m, 2H), 3.84 (s, 3H), 3.47-3.40 (m, 2H), 3.06-2.95 (m, 1H), 1.83-1.65 (m, 4H).

### Step 5: Methyl 4-(2-(2-aminopyridin-3-yl)-5-(tetrahydro-2H-pyran-4-yl)-3H-imidazo[4,5-b]pyridin-3-yl)benzoate

To a solution of methyl 4-((3-nitro-6-(tetrahydro-2H-pyran-4-yl) pyridin-2-yl) amino) benzoate (0.5 g, 1.4 mmol) in DMSO (30 mL) were added Na₂S₂O₄ (974 1 mg, 5.6 mmol) and 2-aminonicotinaldehyde (205 mg, 1.7 mmol). The mixture was stirred at 100° C. for 16 hr. The reaction mixture was poured into H₂O (100 mL), extracted with CH₂Cl₂ (100 mL×2). The organic phase was dried over anhydrous Na₂SO₄, filtered and concentrated under reduced pressure. The crude product was purified by silica gel flash chromatography (Eluent of 0˜76% EtOAc in petroleum ether) to give methyl 4-(2-(2-aminopyridin-3-yl)-5-(tetrahydro-2H-pyran-4-yl)-3H-imidazo[4,5-b]pyridin-3-yl)benzoate (200 mg, yield: 33%) as a yellow solid. MS: m/z=430.0 [M+H]⁺. ¹H NMR (400 MHz, Chloroform-d) δ 8.20-8.17 (m, 2H), 8.06 (dd, J=4.8, 1.6 Hz, 1H), 8.03 (d, J=8.4 Hz, 1H), 7.51-7.47 (m, 2H), 7.22 (d, J=8.4 Hz, 1H), 7.04 (dd, J=7.6, 1.6 Hz, 1H), 6.61 (br s, 2H), 6.39 (dd, J=8.0, 4.8 Hz, 1H), 4.09-4.05 (m, 2H), 3.97 (s, 3H), 3.57-3.51 (m, 2H), 3.05-2.97 (m, 1H), 1.95-1.83 (m, 4H).

### Step 6: (4-(2-(2-Aminopyridin-3-yl)-5-(tetrahydro-2H-pyran-4-yl)-3H-imidazo[4,5-b]pyridin-3-yl)phenyl))methanol

To a solution of methyl 4-(2-(2-aminopyridin-3-yl)-5-(tetrahydro-2H-pyran-4-yl)-3H-imidazo[4,5-b]pyridin-3-yl)benzoate (200 mg, 466 μmol) in THF (10 mL) was added dropwise LiAlH₄ (2.5 M, 372 μL) at 0° C. The resulting mixture was stirred at 25° C. for 1 hr. After the reaction mixture was cooled to 0° C., the reaction mixture was quenched with H₂O (15 mL), followed by 15% aqueous NaOH (10 mL). After being stirred at room temperature for 30 min, the mixture was filtered through celite. The filtrate was concentrated to dryness to give (4-(2-(2-aminopyridin-3-yl)-5-(tetrahydro-2H-pyran-4-yl)-3H-imidazo[4,5-b]pyridin-3-yl)phenyl)methanol (190 mg, crude) as a light yellow solid, which was used to the next step directly. MS: m/z=402.2 [M+H]⁺

### Step 7: 3-(3-(4-(Chloromethyl)phenyl)-5-(tetrahydro-2H-pyran-4-yl)-3H-imidazo[4,5-b]pyridin-2-yl)pyridin-2-amine

To a solution of (4-(2-(2-aminopyridin-3-yl)-5-(tetrahydro-2H-pyran-4-yl)-3H-imidazo[4,5-b]pyridin-3-yl)phenyl)methanol (190 mg, 473 μmol) in CH₂Cl₂ (5 mL) was added SOCl₂ (1.6 g, 13.1 mmol). The mixture was stirred at 40° C. for 1 hr. The reaction mixture was concentrated under reduced pressure to give 3-(3-(4-(chloromethyl)phenyl)-5-(tetrahydro-2H-pyran-4-yl)-3H-imidazo[4,5-b]pyridin-2-yl)pyridin-2-amine (Intermediate 82, 200 mg, crude) as a brown solid, which was used to the next step directly. MS: m/z=420.0 [M+H]⁺

### Intermediate 83: 4-(Azetidin-3-ylamino)-1,3,5-triazine-2-carbonitrile

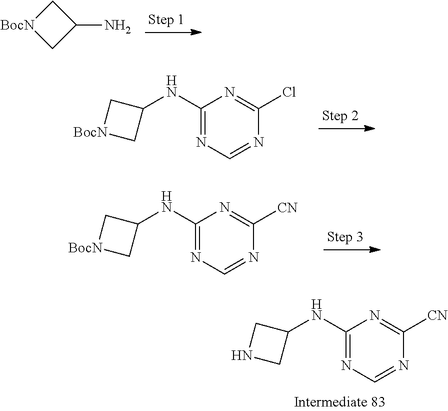 [SMILES: CC(C)(C)OC(=O)N1CC(N)C1 ; CC(C)(C)OC(=O)N1CC(Nc2ncnc(Cl)n2)C1 ; CC(C)(C)OC(=O)N1CC(Nc2ncnc(C#N)n2)C1 ; N#Cc1ncnc(NC2CNC2)n1]

### Step 1: tert-Butyl 3-((4-chloro-1,3,5-triazin-2-yl)amino)azetidine-1-carboxylate

To a solution of tert-butyl 3-aminoazetidine-1-carboxylate (500 mg, 2.50 mmol) in THF (15 mL) was added DIEA (562 mg, 4.35 mmol) at 0° C. A solution of 2,4-dichloro-1,3,5-triazine (435 mg, 2.90 mmol) in THF (5 mL) was added to the mixture 10 minutes later. The mixture was stirred at 0° C. for 4 hr. The reaction mixture was diluted with water (20 mL) and extracted with EtOAc (30 mL×3). The combined organic layers were dried over Na₂SO₄, filtered and concentrated under reduced pressure. The residue was purified by silica gel flash chromatography (Eluent of 0˜30% EtOAc in petroleum ether) to give tert-butyl 3-((4-chloro-1,3,5-triazin-2-yl)amino)azetidine-1-carboxylate (250 mg, yield: 31%) as a colorless oil. MS: m/z=286.1 [M+H]⁺. ¹H NMR (400 MHz, Chloroform-d) δ 8.49-8.32 (m, 1H), 6.25-6.00 (m, 1H), 4.85-4.66 (m, 1H), 4.40-4.25 (m, 2H), 3.90-3.75 (m, 2H), 1.45 (s, 9H).

### Step 2: tert-Butyl 3-((4-cyano-1,3,5-triazin-2-yl)amino)azetidine-1-carboxylate

To a solution of tert-butyl 3-((4-chloro-1,3,5-triazin-2-yl)amino)azetidine-1-carboxylate (350 mg, 1.22 mmol) in DMSO (3 mL) was added KCN (159 mg, 2.45 mmol), the mixture was stirred 0.5 hr at 25° C. Then DABCO (27.5 mg, 244 μmol) was added to the reaction mixture. The reaction mixture was stirred at 25° C. for 1 hr. The reaction mixture was poured into water (20 mL). The resulting mixture was extracted with EtOAc (20 mL×2). The combined organic layers were washed with brine (20 ml) and dried over anhydrous Na₂SO₄, filtered and concentrated to dryness. The residue was purified by silica gel flash chromatography (Eluent of 0˜40% EtOAc in petroleum ether) to give tert-butyl 3-((4-cyano-1,3,5-triazin-2-yl)amino)azetidine-1-carboxylate (140 mg, yield: 33%) as a yellow solid. MS: m/z=299.1 [M+Na]˜. ¹H NMR (400 MHz, Chloroform-d) δ 8.75-8.53 (m, 1H), 6.24-5.96 (m, 1H), 4.81-4.64 (m, 1H), 4.45-4.23 (m, 2H), 3.95-3.75 (m, 2H), 1.45 (s, 9H).

### Step 3: 4-(Azetidin-3-ylamino)-1,3,5-triazine-2-carbonitrile

To a solution of tert-butyl 3-((4-cyano-1,3,5-triazin-2-yl)amino)azetidine-1-carboxylate (140 mg, 506 μmol) in CH₂Cl₂ (2 mL) was added TFA (57.7 mg, 506 μmol). The mixture was stirred at 25° C. for 1 hr. The mixture was concentrated under reduced pressure to give 4-(azetidin-3-ylamino)-1,3,5-triazine-2-carbonitrile (Intermediate 83, 130 mg, TFA salt) as a yellow oil.

### Intermediate 84: 4-(2,6-Diazaspiro[3.4]octan-2-yl)-1,3,5-triazine-2-carbonitrile

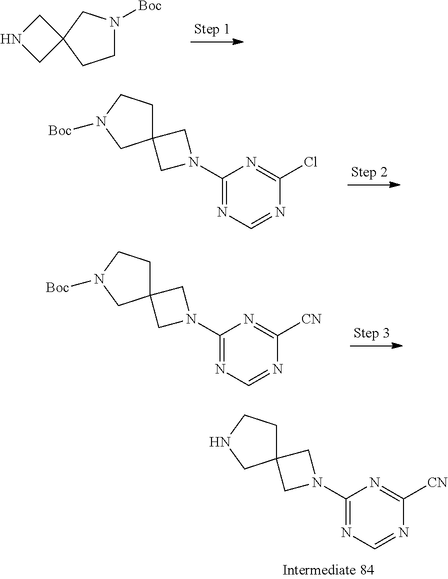

### Step 1: Tert-butyl 2-(4-chloro-1,3,5-triazin-2-yl)-2,6-diazaspiro[3.4]octane-6-carboxylate

To a solution of tert-butyl 2,6-diazaspiro[3.4]octane-6-carboxylate (300 mg, 1.4 mmol) in THF (5 mL) was added DIEA (548 mg, 4.3 mmol) at 25° C., then 2,4-dichloro-1,3,5-triazine (212 mg, 1.4 mmol) in THF (5 mL) was added into the mixture at 0° C. The mixture was stirred at 25° C. for 12 hr under N₂. The reaction mixture was poured into H₂O (50 mL). The mixture was extracted with CH₂Cl₂ (20 mL×2). The combined organic layers were washed with brine (40 mL), dried over anhydrous Na₂SO₄, filtered and concentrated to dryness. The residue was purified by silica gel flash chromatography (Eluent of 0˜30% EtOAc in petroleum ether) to give tert-butyl 2-(4-chloro-1,3,5-triazin-2-yl)-2,6-diazaspiro[3.4]octane-6-carboxylate (230 mg, yield: 47%) as a yellow solid. MS: m/z=326.1 [M+H]+. ¹H NMR (400 MHz, Dimethylsulfoxide-d₆) δ 8.44 (s, 1H), 4.16-3.98 (m, 4H), 3.47-3.41 (m, 2H), 3.31-3.25 (m, 2H), 2.11-2.05 (m, 2H), 1.40 (s, 9H).

### Step 2: Tert-butyl 2-(4-cyano-1,3,5-triazin-2-yl)-2,6-diazaspiro[3.4]octane-6-carboxylate

To a solution of tert-butyl 2-(4-chloro-1,3,5-triazin-2-yl)-2,6-diazaspiro[3.4]octane-6-carboxylate (200 mg, 613 μmol) in DMSO (5 mL) were added KCN (140 mg, 2.2 mmol) and DABCO (14 mg, 123 μmol) at 25° C. The mixture was stirred at 25° C. for 2 hr. The reaction mixture was poured into H₂O (20 mL). The mixture was extracted with EtOAc (20 mL×2). The combined organic layers were washed with brine (40 mL), dried over anhydrous Na₂SO₄, filtered and concentrated to dryness. The residue was purified by silica gel flash chromatography (Eluent of 0˜30% EtOAc in petroleum ether) to give tert-butyl 2-(4-cyano-1,3,5-triazin-2-yl)-2,6-diazaspiro[3.4]octane-6-carboxylate (150 mg, yield: 76%) as a yellow solid. MS: m/z=317.0 [M+H]⁺.

### Step 3: 4-(2,6-Diazaspiro[3.4]octan-2-yl)-1,3,5-triazine-2-carbonitrile

To a solution of tert-butyl 2-(4-cyano-1,3,5-triazin-2-yl)-2,6-diazaspiro[3.4]octane-6-carboxylate (150 mg, 474 μmol) in CH₂Cl₂ (3 mL) was added TFA (1.5 g, 13 mmol) at 25° C. The mixture was stirred at 25° C. for 2 hr. The reaction mixture was concentrated under reduced pressure to give a crude 4-(2,6-diazaspiro[3.4]octan-2-yl)-1,3,5-triazine-2-carbonitrile (Intermediate 84, 130 mg, TFA salt) as a yellow solid, which was used in the next step without further purification.

### Intermediate 85: 4-(2,6-Diazaspiro[3.3]heptan-2-yl)-1,3,5-triazine-2-carbonitrile

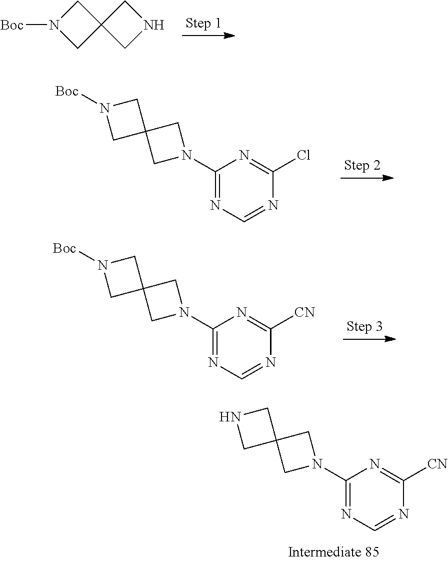

### Step 1: tert-Butyl 6-(4-chloro-1,3,5-triazin-2-yl)-2,6-diazaspiro[3.3]heptane-2-carboxylate

To a solution of tert-butyl 2,6-diazaspiro[3.3]heptane-2-carboxylate (300 mg, 1.5 mmol) in THE (5 mL) was added DIEA (587 mg, 4.5 mmol) at 25° C., then a solution of 2,4-dichloro-1,3,5-triazine (227 mg, 1.5 mmol) in THE (5 mL) was added into the mixture at 0° C. The mixture was stirred at 25° C. for 12 hr under N₂. The reaction mixture was poured into H₂O (50 mL). The mixture was extracted with CH₂Cl₂ (20 mL×2). The combined organic layers were washed with brine (40 mL), dried over anhydrous Na₂SO₄, filtered and concentrated to dryness. The residue was purified by silica gel flash chromatography (Eluent of 0˜30% EtOAc in petroleum ether) to give tert-butyl 6-(4-chloro-1,3,5-triazin-2-yl)-2,6-diazaspiro[3.3]heptane-2-carboxylate (200 mg, yield: 42%) as a yellow solid. MS: m/z=312.1 [M+H]⁺. ¹H NMR (400 MHz, Dimethylsulfoxide-d₆) δ 8.44 (s, 1H), 4.28 (d, J=4.8 Hz, 4H), 4.03 (s, 4H), 1.38 (s, 9H).

### Step 2: tert-Butyl 6-(4-cyano-1,3,5-triazin-2-yl)-2,6-diazaspiro[3.3]heptane-2-carboxylate

To a solution of tert-butyl 6-(4-chloro-1,3,5-triazin-2-yl)-2,6-diazaspiro[3.3]heptane-2-carboxylate (191 mg, 614 μmol) in DMSO (5 mL) were added KCN (160 mg, 2.5 mmol) and DABCO (14 mg, 123 μmol) at 25° C. The mixture was stirred at 25° C. for 2 hr. The reaction mixture was poured into H₂O (20 mL), and extracted with EtOAc (20 mL×2). The combined organic layers were washed with brine (40 mL), dried over anhydrous Na₂SO₄, filtered and concentrated to dryness. The residue was purified by silica gel flash chromatography (Eluent of 0˜30% EtOAc in petroleum ether) to give tert-butyl 6-(4-cyano-1,3,5-triazin-2-yl)-2,6-diazaspiro[3.3]heptane-2-carboxylate (150 mg, yield: 70%) was obtained as a yellow solid. MS: m/z=303.0 [M+H]⁺.

### Step 3: 4-(2,6-Diazaspiro[3.3]heptan-2-yl)-1,3,5-triazine-2-carbonitrile

To a solution of tert-butyl 6-(4-cyano-1,3,5-triazin-2-yl)-2,6-diazaspiro[3.3]heptane-2-carboxylate (150 mg, 496 μmol) in CH₂Cl₂ (3 mL) was added TFA (1.6 g, 14 mmol) at 25° C., the mixture was stirred at 25° C. for 2 hr. The reaction mixture was concentrated under reduced pressure to give a crude product 4-(2,6-diazaspiro[3.3]heptan-2-yl)-1,3,5-triazine-2-carbonitrile (Intermediate 85, 130 mg, TFA salt) as a yellow solid, which was used in the next step without further purification.

### Intermediate 86: 2-(Piperazin-1-yl)pyrimidine-4-carbonitrile

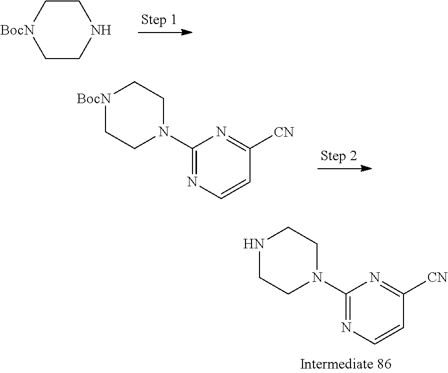 [SMILES: CC(C)(C)OC(=O)N1CCNCC1 ; CC(C)(C)OC(=O)N1CCN(c2nccc(C#N)n2)CC1 ; N#Cc1ccnc(N2CCNCC2)n1]

### Step 1: tert-Butyl 4-(4-cyanopyrimidin-2-yl)piperazine-1-carboxylate

To a solution of 2-chloropyrimidine-4-carbonitrile (2 g, 14.3 mmol) in MeCN (20 mL) were added K₂CO₃ (5.94 g, 43 mmol), tert-butyl piperazine-1-carboxylate (2.67 g, 14.3 mmol) and NaI (430 mg, 2.87 mmol). The mixture was stirred at 80° C. for 16 hr. The reaction mixture was diluted with H₂O (50 mL) and EtOAc (50 mL), and then extracted with EtOAc (150 mL×2). The combined organic layers were washed with brine (150 mL), dried over Na₂SO₄, filtered and concentrated under reduced pressure to give tert-butyl 4-(4-cyanopyrimidin-2-yl)piperazine-1-carboxylate (4.2 g, yield: 98%) as a yellow solid. MS: m/z=190.1 [M+H−100]⁺. ¹H NMR (400 MHz, Dimethylsulfoxide-d₆) δ 8.67 (d, J=4.8 Hz, 1H), 7.19 (d, J=4.8 Hz, 1H), 3.77-3.68 (m, 4H), 3.46-3.37 (m, 4H), 1.43 (s, 9H).

### Step 2: 2-(Piperazin-1-yl)pyrimidine-4-carbonitrile

To a solution of tert-butyl 4-(2-cyanopyrimidin-4-yl)piperazine-1-carboxylate (130 mg, 449 μmol) in CH₂Cl₂ (3 mL) was added TFA (717 mg, 6.30 mmol). The mixture was stirred at 25° C. for 1 hr. The reaction mixture was concentrated under reduced pressure. The crude 2-(piperazin-1-yl)pyrimidine-4-carbonitrile (Intermediate 86, 137 mg, TFA salt, yield: 100%) was used in the next step without further purification.

### Intermediate 87: 2-(Piperidin-4-ylamino)pyrimidine-4-carbonitrile

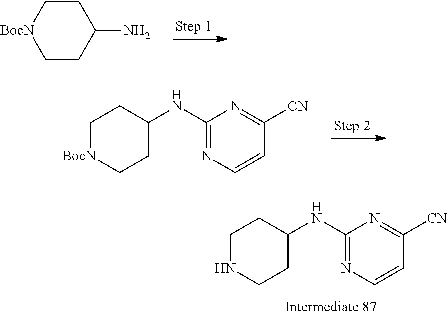 [SMILES: CC(C)(C)OC(=O)N1CCC(N)CC1 ; CC(C)(C)OC(=O)N1CCC(Nc2nccc(C#N)n2)CC1 ; N#Cc1ccnc(NC2CCNCC2)n1]

### Step 1: tert-Butyl 4-((4-cyanopyrimidin-2-yl)amino)piperidine-1-carboxylate

To a solution of 2-chloropyrimidine-4-carbonitrile (2 g, 14.3 mmol) in MeCN (20 mL) were added K₂CO₃ (5.94 g, 43 mmol), tert-butyl 4-aminopiperidine-1-carboxylate (2.87 g, 14.3 mmol) and NaI (430 mg, 2.87 mmol). The mixture was stirred at 80° C. for 16 hr. The reaction mixture was diluted with H₂O (50 mL) and EtOAc (50 mL), and then extracted with EtOAc (150 mL×2). The combined organic layers were washed with aqueous NaCl (150 mL), dried over Na₂SO₄, filtered and concentrated under reduced pressure to give tert-butyl 4-((4-cyanopyrimidin-2-yl)amino)piperidine-1-carboxylate (4.5 g, yield: 98%) as a yellow solid. MS: m/z=248.1 [M+H−56]⁺. ¹H NMR (400 MHz, Dimethylsulfoxide-d₆) δ 8.61 (d, 0.1=4.4 Hz, 1H), 7.10 (d, J=4.8 Hz, 1H), 6.86 (d, J=7.6 Hz, 1H), 4.50-4.41 (m, 2H), 3.62-3.47 (m, 1H), 3.12-3.03 (m, 2H), 1.84-1.75 (m, 2H), 1.39 (s, 9H), 1.33-1.24 (m, 2H).

### Step 2: 2-(Piperidin-4-ylamino)pyrimidine-4-carbonitrile

To a solution of tert-butyl 4-((4-cyanopyrimidin-2-yl)amino)piperidine-1-carboxylate (140 mg, 483 μmol) in CH₂Cl₂ (3 mL) was added TFA (767 mg, 6.73 mmol). The mixture was stirred at 25° C. for 1 hr. The reaction mixture was concentrated under reduced pressure. The crude product 2-(piperidin-4-ylamino)pyrimidine-4-carbonitrile (Intermediate 87, 146 mg TFA salt, yield: 100%) was used in the next step without further purification. MS: m/z=204.0 [M+H]⁺.

### Intermediate 88: (R)-4-(Piperidin-3-ylamino)-1,3,5-triazine-2-carbonitrile

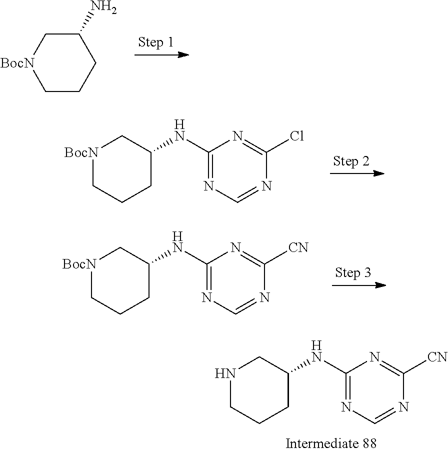

### Step 1: Tert-butyl (R)-3-((4-chloro-1,3,5-triazin-2-yl)amino)piperidine-1-carboxylate

To a solution of tert-butyl (R)-3-aminopiperidine-1-carboxylate (500 mg, 2.50 mmol) in THF (15 mL) was added DIEA (483 mg, 3.74 mmol) at 0° C., the mixture was stirred 10 min followed by addition of a solution of 2,4-dichloro-1,3,5-triazine (374 mg, 2.50 mmol) in THF (5 mL). The mixture was stirred at 0° C. for 4 hr. The reaction mixture was diluted with water (20 mL) and extracted with EtOAc (30 mL×3). The combined organic layers were dried over Na₂SO₄, filtered and concentrated under reduced pressure. The residue was purified by silica gel flash chromatography (Eluent of 0˜30% EtOAc in petroleum ether) to give tert-butyl (R)-3-((4-chloro-1,3,5-triazin-2-yl)amino)piperidine-1-carboxylate (250 mg, yield: 31%) as a colorless oil. MS: m/z=314.1 [M+H]f ¹H NMR (400 MHz, Chloroform-d) S 8.52-8.25 (m, 1H), 5.88-5.61 (m, 1H), 4.17-3.96 (m, 1H), 3.68-3.28 (m, 4H), 1.98-1.85 (m, 1H), 1.77-1.59 (m, 3H), 1.44 (s, 9H).

### Step 2: Tert-butyl (R)-3-((4-cyano-1,3,5-triazin-2-yl)amino)piperidine-1-carboxylate

To a solution of tert-butyl (R)-3-((4-chloro-1,3,5-triazin-2-yl)amino)piperidine-1-carboxylate (250 mg, 796 mmol) in DMSO (5 mL) was added KCN (60 mg, 921 mmol), the mixture was stirred 0.5 hr at 25° C. DABCO (17.9 mg, 159 μmol) was added to the reaction mixture. The reaction mixture was stirred at 25° C. for 1 hr. The reaction mixture was poured into water (20 mL). The resulting mixture was extracted with EtOAc (20 mL×2). The combined organic layers were washed with brine (20 mL) and dried over anhydrous Na₂SO₄, filtered and concentrated to dryness. The residue was purified by silica gel flash chromatography (Eluent of 0˜29% EtOAc in petroleum ether) to give tert-butyl (R)-3-((4-cyano-1,3,5-triazin-2-yl)amino)piperidine-1-carboxylate (135 mg, yield: 51%) was obtained as a yellow solid. MS: m/z=305.1 [M+H]⁺. ¹H NMR (400 MHz, Chloroform-d) δ 8.66-8.54 (m, 1H), 6.02-5.75 (m, 1H), 4.10-4.01 (m, 1H), 3.62-3.28 (m, 4H), 1.97-1.85 (m, 1H), 1.83-1.62 (m, 3H), 1.45 (s, 9H).

### Step 3: (R)-4-(Piperidin-3-ylamino)-1,3,5-triazine-2-carbonitrile

To a solution of tert-butyl (R)-3-((4-cyano-1,3,5-triazin-2-yl)amino)piperidine-1-carboxylate (135 mg, 443 μmol) in CH₂Cl₂ (2 mL) was added TFA (50.5 mg, 443 μmol). The mixture was stirred at 25° C. for 1 hr. The mixture was concentrated under reduced pressure to give (R)-4-(piperidin-3-ylamino)-1,3,5-triazine-2-carbonitrile (Intermediate 88, 130 mg, TFA salt used directly) as a yellow oil.

### Intermediate 89: 3-(3-(4-(Chloromethyl)phenyl)-5-(3-fluorophenyl)-3H-imidazo[4,5-b]pyridin-2-yl)pyridin-2-amine

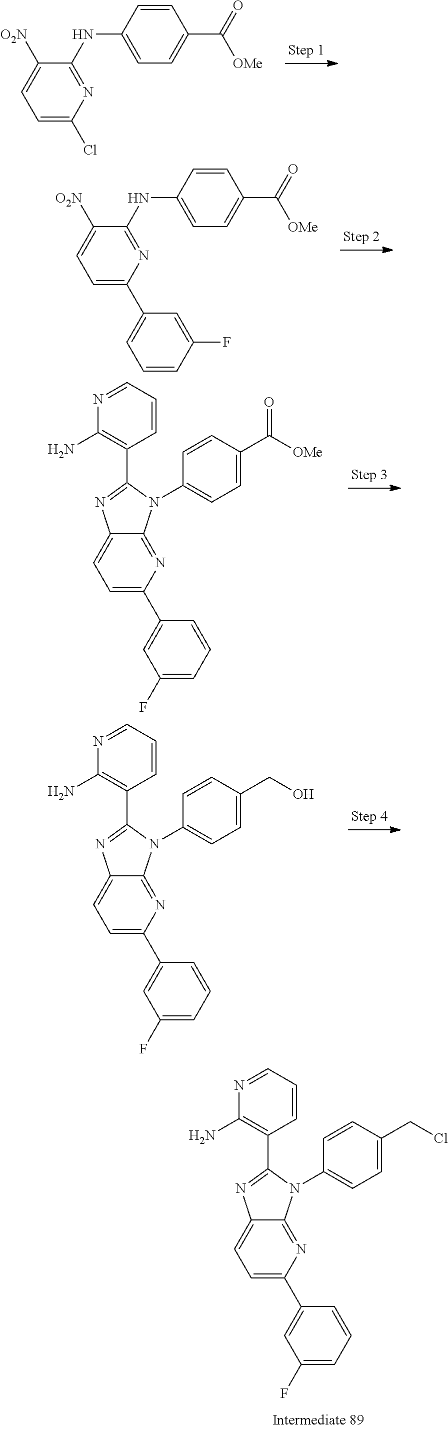

### Step 1: Methyl 4-((6-(3-fluorophenyl)-3-nitropyridin-2-yl)amino)benzoate

To a mixture of methyl 4-((6-chloro-3-nitropyridin-2-yl)amino)benzoate (refer to Intermediate 13 for detail procedures, 5.0 g, 16.2 mmol), (3-fluorophenyl)boronic acid (2.96 g, 21.1 mmol), Pd(dppf)Cl₂ (1.19 g, 1.63 mmol), Cs₂CO₃ (15.8 g, 48.7 mmol) in 1,4-dioxane (50 mL) and H₂O (5 mL) was degassed and purged with N₂ three times, and the mixture was stirred at 100° C. for 12 hr under N₂ atmosphere. The reaction mixture was diluted with water (20 mL) and extracted with CH₂Cl₂ (30 mL/3). The combined organic layers were dried over Na₂SO₄, filtered and concentrated under reduced pressure. The residue was purified by silica gel flash chromatography (Eluent of 0˜10% MeOH in CH₂Cl₂) to give methyl 4-((6-(3-fluorophenyl)-3-nitropyridin-2-yl)amino)benzoate (4.5 g, yield: 67%) as a yellow solid. MS: m/z=367.9 [M+H]⁺. ¹H NMR (400 MHz, Chloroform-d) δ 10.44 (br s, 1H), 8.64 (d, J=8.4 Hz, 1H), 8.13 (d, J=8.0 Hz, 2H), 7.92-7.80 (m, 3H), 7.77 (d, J=10.0 Hz, 1H), 7.55-7.46 (m, 1H), 7.37 (d, J=8.8 Hz, 1H), 7.26-7.19 (m, 1H), 3.95 (s, 3H), ¹⁹F NMR (400 MHz, Chloroform-d) δ −111.554.

### Step 2: Methyl 4-(2-(2-aminopyridin-3-yl)-5-(3-fluorophenyl)-3H-imidazo[4,5-b]pyridin-3-yl)benzoate

To a solution of methyl 4-((6-(3-fluorophenyl)-3-nitropyridin-2-yl)amino)benzoate (4.3 g, 11.7 mmol) and 2-aminonicotinaldehyde (1.57 g, 12.9 mmol) in DMSO (100 mL) was added Na₂S₂O₄ (9.59 g, 46.8 mmol). The mixture was stirred at 100° C. for 12 hr. The reaction mixture was diluted with water (100 mL) and extracted with CH₂Cl₂ (300 mL×2). The combined organic layers were washed with brine (100 mL), dried over Na₂SO₄, filtered and concentrated under reduced pressure. The residue was purified by silica gel flash chromatography (Eluent of 0˜100% EtOAc in petroleum ether) to give methyl 4-(2-(2-aminopyridin-3-yl)-5-(3-fluorophenyl)-3H-imidazo[4,5-b]pyridin-3-yl)benzoate (3.0 g, yield: 41%) as a yellow solid. MS: m/z=440.1 [M+H]⁺. ¹H NMR (400 MHz, Chloroform-d) δ 8.22 (d, J=8.4 Hz, 2H), 8.17-8.06 (m, 2H), 7.83-7.69 (m, 3H), 7.55 (d, J=8.4 Hz, 2H), 7.45-7.35 (m, 1H), 7.11-7.02 (m, 2H), 6.63 (br s, 2H), 6.39 (dd, J=7.6, 5.2 Hz, 1H), 3.98 (s, 3H).

### Step 3: (4-(2-(2-Aminopyridin-3-yl)-5-(3-fluorophenyl)-3H-imidazo[4,5-b]pyridin-3-yl)phenyl)methanol

To a solution of methyl 4-(2-(2-aminopyridin-3-yl)-5-(3-fluorophenyl)-3H-imidazo[4,5-b]pyridin-3-yl)benzoate (3 g, 6.83 mmol) in THF (125 mL) was added LiAlH₄ (2.5 M, 4.10 mL) at 0° C. The mixture was stirred at 25° C. for 2 hr. The reaction mixture was quenched with Na₂SO₄·10H₂O (120 mg) at 0° C. and filtered. The filtrate was concentrated under reduced pressure to give (4-(2-(2-aminopyridin-3-yl)-5-(3-fluorophenyl)-3H-imidazo[4,5-b]pyridin-3-yl)phenyl)methanol (3 g, crude) as a yellow solid.

### Step 4: 3-(3-(4-(Chloromethyl)phenyl)-5-(3-fluorophenyl)-3H-imidazo[4,5-b]pyridin-2-yl)pyridin-2-amine

To a solution of (4-(2-(2-aminopyridin-3-yl)-5-(3-fluorophenyl)-3H-imidazo[4,5-b]pyridin-3-yl)phenyl)methanol (3 g, 7.29 mmol) in CH₂Cl₂ (20 mL) was added SOCl₂ (5.20 g, 43.7 mmol). The mixture was stirred at 40° C. for 2 hr. The mixture was concentrated under reduced pressure to give 3-(3-(4-(chloromethyl)phenyl)-5-(3-fluorophenyl)-3H-imidazo[4,5-b]pyridin-2-yl)pyridin-2-amine (Intermediate 89, 3.4 g, HCl salt) as yellow solid. MS: m/z=430.0 [M+H]⁺.

### Intermediate 90: 3-(3-(4-(Chloromethyl)phenyl)-5-(2-fluorophenyl)-3H-imidazo[4,5-b]pyridin-2-yl)pyridin-2-amine

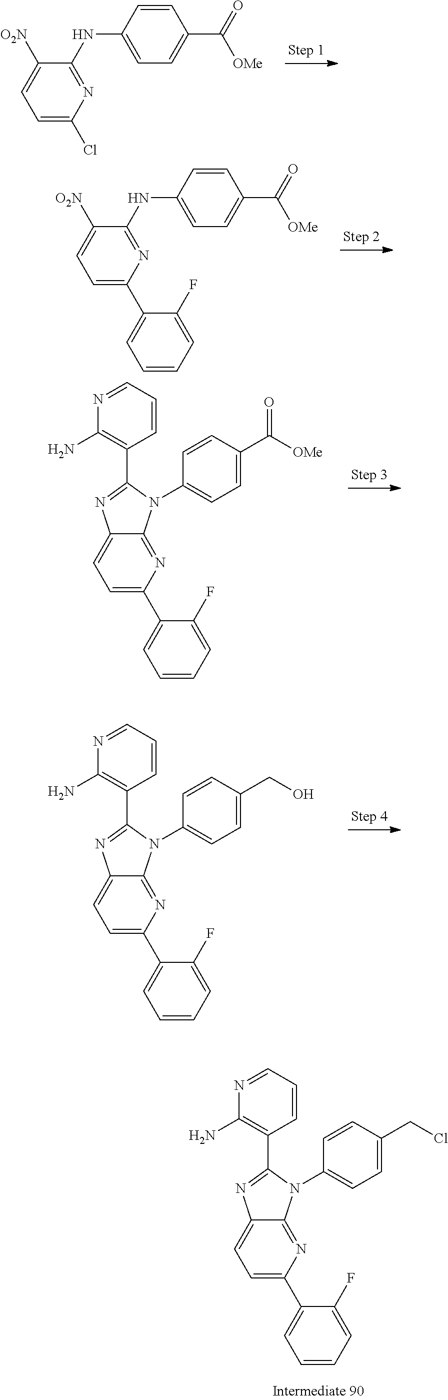

### Step 1: Methyl 4-((6-(2-fluorophenyl)-3-nitropyridin-2-yl)amino)benzoate

To a mixture of methyl 4-((6-chloro-3-nitropyridin-2-yl)amino)benzoate (refer to Intermediate 13 for detail procedures, 5 g, 16.2 mmol), (2-fluorophenyl)boronic acid (2.9 g, 21.1 mmol), Pd(dppf)Cl₂ (1.19 g, 1.63 mmol), Cs₂CO₃ (15.8 g, 48.7 mmol) in 1,4-dioxane (50 mL) and H₂O (5 mL) was degassed and purged with N₂ three times, and the mixture was stirred at 100° C. for 12 hr under N₂ atmosphere. The reaction mixture was diluted with water (20 mL) and extracted with CH₂Cl₂ (30 mL×2). The combined organic layers were dried over Na₂SO₄, filtered and concentrated under reduced pressure. The residue was purified by silica gel flash silica gel flash chromatography (Eluent of 0˜10% MeOH in CH₂Cl₂) to give methyl 4-((6-(2-fluorophenyl)-3-nitropyridin-2-yl)amino)benzoate (4.5 g, yield: 73%) as a yellow solid. MS: m/z=368.0 [M+H]⁺. ¹H NMR (400 MHz, Chloroform-d) δ 10.44 (s, 1H), 8.62 (d, J=8.4 Hz, 1H), 8.11-8.07 (m, 2H), 8.07-8.02 (m, 1H), 7.90-7.84 (m, 2H), 7.54-7.46 (m, 2H), 7.34-7.29 (m, 1H), 7.25-7.19 (m, 1H), 3.94 (s, 3H)

### Step 2: Methyl 4-(2-(2-aminopyridin-3-yl)-5-(2-fluorophenyl)-3H-imidazo[4,5-b]pyridin-3-yl)benzoate

To a solution of methyl 4-((6-(2-fluorophenyl)-3-nitropyridin-2-yl)amino)benzoate (4.5 g, 12.2 mmol) and 2-aminonicotinaldehyde (1.65 g, 13.5 mmol) in DMSO (100 mL) was added Na₂S₂O₄ (10.0 g, 49.0 mmol). The mixture was stirred at 100° C. for 12 hr. The reaction mixture was diluted with water (100 mL) and extracted with CH₂Cl₂ (300 mL×2). The combined organic layers were washed with brine (100 mL), dried over Na₂SO₄, filtered and concentrated under reduced pressure. The residue was purified by silica gel flash chromatography (Eluent of 0˜100% EtOAc in petroleum ether) to give methyl 4-(2-(2-aminopyridin-3-yl)-5-(2-fluorophenyl)-3H-imidazo[4,5-b]pyridin-3-yl)benzoate (2.5 g, yield: 45%) as a yellow solid. MS: m/z=440.1 [M+H]⁺. ¹H NMR (400 MHz, Chloroform-d) δ 8.22 (d, J=8.4 Hz, 2H), 8.17 (d, J=8.4 Hz, 1H), 8.09 (dd, J=5.2, 2.0 Hz, 1H), 7.98-7.90 (m, 2H), 7.56 (d, J=8.4 Hz, 2H), 7.39-7.33 (m, 1H), 7.24-7.09 (m, 3H), 6.80 (br s, 2H), 6.42 (dd, J=7.6, 4.8 Hz, 1H), 3.98 (s, 3H).

### Step 3: (4-(2-(2-Aminopyridin-3-yl)-5-(2-fluorophenyl)-3H-imidazo[4,5-b]pyridin-3-yl)phenyl)methanol

To a solution of methyl 4-(2-(2-aminopyridin-3-yl)-5-(2-fluorophenyl)-3H-imidazo[4,5-b]pyridin-3-yl)benzoate (2.5 g, 5.69 mmol) in THF (100 mL) was added LiAlH₄ (2.5 M, 3.41 mL) at 0° C. The mixture was stirred at 25° C. for 2 hr. The reaction mixture was quenched with Na₂SO₄—H₂O (389 mg) at 0° C. and filtered. The filtrate was concentrated under reduced pressure to give (4-(2-(2-aminopyridin-3-yl)-5-(2-fluorophenyl)-3H-imidazo[4,5-b]pyridin-3-yl)phenyl)methanol (1.5 g, crude) as a yellow solid.

### Step 4: 3-(3-(4-(Chloromethyl)phenyl)-5-(2-fluorophenyl)-3H-imidazo[4,5-b]pyridin-2-yl)pyridin-2-amine

To a solution of (4-(2-(2-aminopyridin-3-yl)-5-(2-fluorophenyl)-3H-imidazo[4,5-b]pyridin-3-yl)phenyl)methanol (1.5 g, 3.65 mmol) in CH₂Cl₂ (20 mL) was added SOCl₂ (2.60 g, 21.9 mmol). The mixture was stirred at 40° C. for 2 hr. The mixture was concentrated under reduced pressure to give 3-(3-(4-(chloromethyl)phenyl)-5-(2-fluorophenyl)-3H-imidazo[4,5-b]pyridin-2-yl)pyridin-2-amine (Intermediate 90, 1.6 g, HCl salt) was obtained as a yellow solid. MS: m/z=430.1 [M+H]⁺.

### Intermediate 91: 4-(2-(2-Aminopyridin-3-yl)-3-(4-(chloromethyl)phenyl)-3H-imidazo[4,5-b]pyridin-5-yl)-1,4-diazepan-2-one

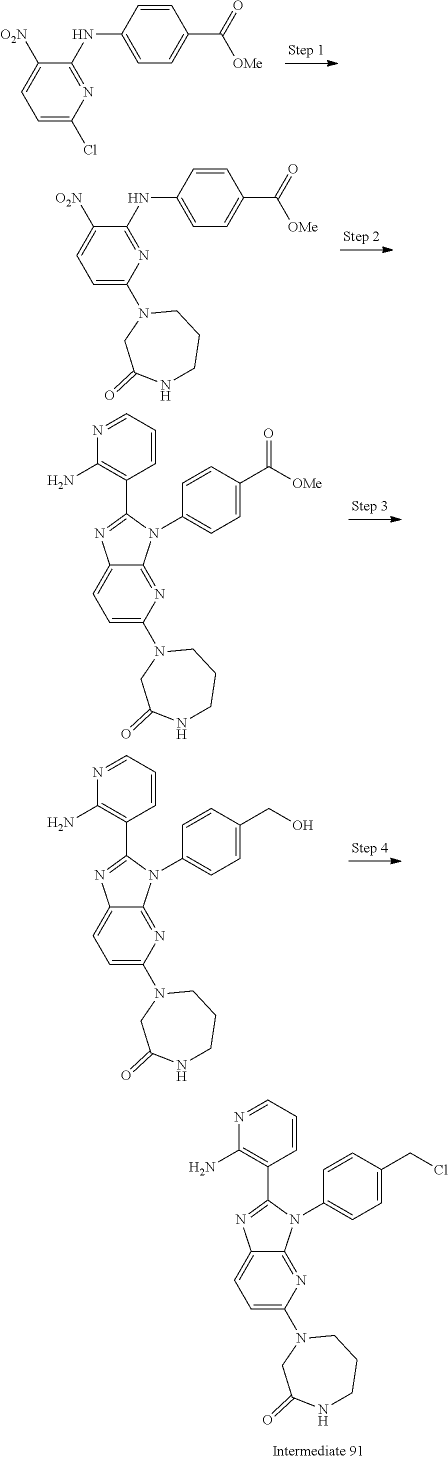

### Step 1: Methyl 4-((3-nitro-6-(3-oxo-1,4-diazepan-1-yl)pyridin-2-yl)amino)benzoate

To a solution of methyl 4-[(6-chloro-3-nitro-2-pyridyl)amino]benzoate (refer to Intermediate 13 for detail procedures, 1 g, 3.25 mmol) in CH₃CN (10 mL) were added DIEA (1.26 g, 9.75 mmol, 1.70 mL) and 1,4-diazepan-2-one (556 mg, HCl salt, 3.69 mmol). The mixture was stirred at 90° C. for 12 hr. Water (10 mL) was added to the mixture and the mixture was filtrated and the filter cake was dried under reduced pressure to give methyl 4-((3-nitro-6-(3-oxo-1,4-diazepan-1-yl)pyridin-2-yl)amino)benzoate (900 mg, yield: 72%) as a yellow solid. ¹H NMR (400 MHz, Dimethylsulfoxide-d₆) δ 10.85-10.68 (m, 1H), 8.30 (d, J=8.4 Hz, 1H), 8.31-7.64 (m, 5H), 6.60-6.45 (m, J H), 4.46-4.27 (m, 2H), 3.99 (s, 2H), 3.84 (s, 3H), 3.31-3.24 (m, 2H), 1.85-1.65 (m, 2H)).

### Step 2: Methyl 4-(2-(2-aminopyridin-3-yl)-5-(3-oxo-1,4-diazepan-1-yl)-3H-imidazo[4,5-b]pyridin-3-yl)benzoate

To a solution of methyl 4-((3-nitro-6-(3-oxo-1,4-diazepan-1-yl)pyridin-2-yl)amino)benzoate (900 mg, 2.34 mmol) in DMSO (10 mL) were added Na₂S₂O₄ (1.22 g, 7.01 mmol) and 2-aminopyridine-3-carbaldehyde (342 mg, 2.80 mmol). The mixture was stirred at 100° C. for 12 hr. Water (10 mL) was added to the mixture and the mixture was extracted with CH₂Cl₂ (10 mL×2). The combined organic phases were washed with brine (10 mL), dried over anhydrous Na₂SO₄, filtered and concentrated to give methyl 4-(2-(2-aminopyridin-3-yl)-5-(3-oxo-1,4-diazepan-1-yl)-3H-imidazo[4,5-b]pyridin-3-yl)benzoate (1 g, yield: 94%) as a yellow solid. MS: m/z=458.2 [M+H]⁺.

### Step 3: 4-(2-(2-Aminopyridin-3-yl)-3-(4-(hydroxymethyl)phenyl)-3H-imidazo[4,5-b]pyridin-5-yl)-1,4-diazepan-2-one

To a solution of methyl 4-(2-(2-aminopyridin-3-yl)-5-(3-oxo-1,4-diazepan-1-yl)-3H-imidazo[4,5-b]pyridin-3-yl)benzoate (1 g, 2.19 mmol) in THF (50 mL) was added LiAlH₄ (166 mg, 109 μL) at 0° C. under N₂. The reaction mixture was stirred at 20° C. for 12 hr. Na₂SO₄·10H₂O was added in portions at 0° C. until no bubbles formed, and the resulting mixture was stirred for 10 min. Then filtered and the filter cake was washed with THF (10 mL×2). The filtrate was concentrated under reduced pressure to give 4-(2-(2-aminopyridin-3-yl)-3-(4-(hydroxymethyl)phenyl)-3H-imidazo[4,5-b]pyridin-5-yl)-1,4-diazepan-2-one (939 mg, yield: 100%) as a brown oil, which was used in the next step directly. MS: m/z=430.0 [M+H]⁺.

### Step 4; 4-(2-(2-Aminopyridin-3-yl)-3-(4-(chloromethyl)phenyl)-3H-imidazo[4,5-b]pyridin-5-yl)-1,4-diazepan-2-one

To a solution of 4-(2-(2-aminopyridin-3-yl)-3-(4-(hydroxymethyl)phenyl)-3H-imidazo[4,5-b]pyridin-5-yl)-1,4-diazepan-2-one (939 mg, 2.19 mmol) in CH₂Cl₂ (20 mL) was added SOCl₂ (780 mg, 476 μL) at 20° C. The reaction mixture was stirred at 40° C. for 2 hr. The reaction mixture was concentrated to dryness to give 4-(2-(2-aminopyridin-3-yl)-3-(4-(chloromethyl)phenyl)-3H-imidazo[4,5-b]pyridin-5-yl)-1,4-diazepan-2-one (Intermediate 91, 760 mg, yield: 78%) as a brown solid, which was used in the next step directly. MS: m/z=448.1 [M+H]⁺.

### Intermediate 92: 4-(2-(2-Aminopyridin-3-yl)-3-(4-(chloromethyl)phenyl)-3H-imidazo[4,5-b]pyridin-5-yl)benzonitrile

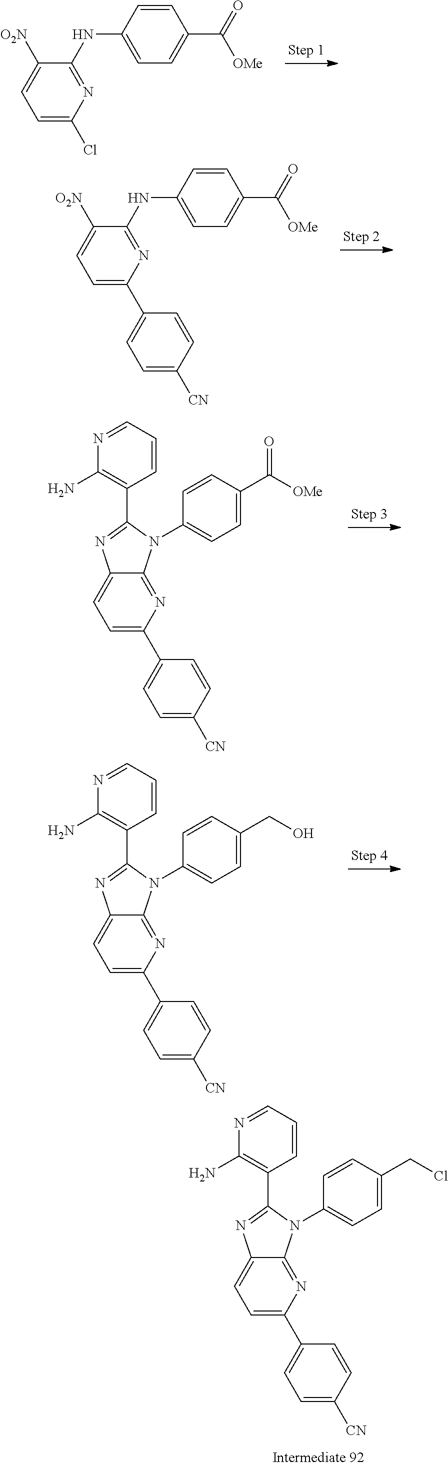

### Step 1: Methyl 4-((6-(4-cyanophenyl)-3-nitropyridin-2-yl)amino)benzoate

A mixture of methyl 4-((6-chloro-3-nitropyridin-2-yl)amino)benzoate (refer to Intermediate 13 for detail procedures, 5 g, 16.3 mmol), (4-cyanophenyl)boronic acid (2.39 g, 16.3 mmol), Cs₂CO₃ (15.9 g, 48.8 mmol), Pd(dppf)Cl₂ (2.38 g, 3.25 mmol) in 1,4-dioxane (100 mL) and H₂O (20 mL) was degassed and purged with N₂ three times, and then the mixture was stirred at 80° C. for 16 hr under N₂ atmosphere. The reaction mixture was diluted with H₂O (100 mL) and extracted with CH₂Cl₂ (50 mL×4). The combined organic layers were washed with brine (30 mL×3), dried over Na₂SO₄, filtered and concentrated under reduced pressure. The residue was purified by silica gel flash chromatography (Eluent of 20˜30% EtOAc in CH₂Cl₂), methyl 4-((6-(4-cyanophenyl)-3-nitropyridin-2-yl)amino)benzoate (6 g, yield: 50%) as a yellow solid. MS: m/z=374.9 [M+H]⁺.

### Step 2: Methyl 4-(2-(2-aminopyridin-3-yl)-5-(4-cyanophenyl)-3H-imidazo[4,5-b]pyridin-3-yl)benzoate

To a solution of methyl 4-((6-(4-cyanophenyl)-3-nitropyridin-2-yl)amino)benzoate (5 g, 13.4 mmol), 2-aminonicotinaldehyde (1.63 g, 13.4 mmol) in DMSO (150 mL) was added Na₂S₂O₄ (9.3 g, 53.4 mmol). The mixture was stirred at 100° C. for 16 hr. The reaction mixture was diluted with H₂O (100 mL) and extracted with CH₂Cl₂ (500 mL×6). The combined organic layers were washed with brine (250 mL×2), dried over Na₂SO₄, filtered and concentrated under reduced pressure to give methyl 4-(2-(2-aminopyridin-3-yl)-5-(4-cyanophenyl)-3H-imidazo[4,5-b]pyridin-3-yl)benzoate (4.8 g, yield: 62%) as a black brown solid, which was directly used in the next step without further purification. MS: m/z=446.9 [M+H]⁺.

### Step 3: 4-(2-(2-Aminopyridin-3-yl)-3-(4-(hydroxymethyl)phenyl)-3H-imidazo[4,5-b]pyridin-5-yl)benzonitrile

To a solution of methyl 4-(2-(2-aminopyridin-3-yl)-5-(4-cyanophenyl)-3H-imidazo[4,5-b]pyridin-3-yl)benzoate (4.8 g, 10.8 mmol) in THF (50 mL) was added LiAlH₄ (2.5 M, 5.16 mL) at 0° C., then the mixture was stirred at 25° C. for 2 hr. The reaction mixture was quenched with Na₂SO₄·10H₂O (8 g) at 0° C., and the mixture was filtered, the filter cake was washed by CH₂Cl₂ (30 mL×3), and the filtrate was concentrated under reduced pressure to give 4-(2-(2-aminopyridin-3-yl)-3-(4-(hydroxymethyl)phenyl)-3H-imidazo[4,5-b]pyridin-5-yl)benzonitrile (4.2 g, yield. 93%) as a yellow solid, which was used in the next step without purification. MS: m/z=419.1 [M+H]⁺.

### Step 4: 4-(2-(2-Aminopyridin-3-yl)-3-(4-(chloromethyl)phenyl)-3H-imidazo[4,5-b]pyridin-5-yl)benzonitrile

To a solution of 4-(2-(2-aminopyridin-3-yl)-3-(4-(hydroxymethyl)phenyl)-3H-imidazo[4,5-b]pyridin-5-yl)benzonitrile (4.2 g, 10.0 mmol) in CH₂Cl₂ (40 mL) was added SOCl₂ (3.58 g, 30.1 mmol) at 0° C. The mixture was stirred at 40° C. for 3 hr. The reaction mixture was quenched with addition NaHCO₃ (5 mL) at 0° C., and then filtered and concentrated under reduced pressure. The residue was purified by silica gel flash chromatography (Eluent of 5˜8% MeOH in CH₂Cl₂), 4-(2-(2-aminopyridin-3-yl)-3-(4-(chloromethyl)phenyl)-3H-imidazo[4,5-b]pyridin-5-yl)benzonitrile (Intermediate 92, 320 mg, yield: 12%) as a yellow solid. MS: m/z=437.0 [M+H]⁺.

### Intermediate 93: 2-(2-(2-Aminopyridin-3-yl)-3-(4-(chloromethyl)phenyl)-3H-imidazo[4,5-b]pyridin-5-yl)benzonitrile

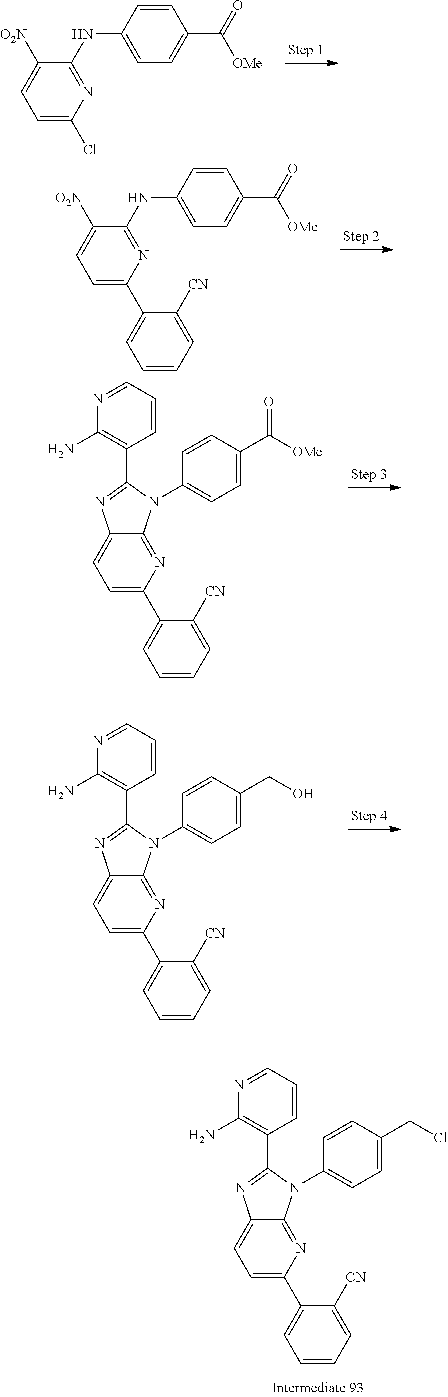

### Step 1: Methyl 4-((6-(2-cyanophenyl)-3-nitropyridin-2-yl)amino)benzoate

A mixture of methyl 4-((6-chloro-3-nitropyridin-2-yl)amino)benzoate (refer to Intermediate 13 for detail procedures, 5 g, 16.3 mmol), (2-cyanophenyl)boronic acid (2.39 g, 16.3 mmol), Cs₂CO₃ (15.9 g, 48.8 mmol), Pd(dppf)Cl₂ (2.38 g, 3.25 mmol) in 1,4-dioxane (100 mL) and H₂O (20 mL) was degassed and purged with N₂ three times, and then the mixture was stirred at 80° C. for 16 hr under N₂ atmosphere. The reaction mixture was diluted with H₂O (100 mL) and extracted with CH₂Cl₂ (50 mL×4). The combined organic layers were washed with brine (30 mL×3), dried over Na₂SO₄, filtered and concentrated under reduced pressure. The residue was purified by silica gel flash chromatography (Eluent of 20˜30% EtOAc in CH₂Cl₂) to give methyl 4-((6-(2-cyanophenyl)-3-nitropyridin-2-yl)amino)benzoate (8.5 g, yield. 71%) as a yellow solid. MS: m/z=374.9 [M+H]⁺.

### Step 2: Methyl 4-(2-(2-aminopyridin-3-yl)-5-(2-cyanophenyl)-3H-imidazo[4,5-b]pyridin-3-yl)benzoate

To a solution of methyl 4-((6-(2-cyanophenyl)-3-nitropyridin-2-yl)amino)benzoate (8 g, 21.4 mmol), 2-aminonicotinaldehyde (2.61 g, 21.4 mmol) in DMSO (200 mL) was added Na₂S₂O₄ (14.9 g, 84.5 mmol). The mixture was stirred at 100° C. for 16 hr. The reaction mixture was diluted with H₂O (100 mL) and extracted with CH₂Cl₂ (500 mL×6). The combined organic layers were washed with brine (250 mL×2), dried over Na₂SO₄, filtered and concentrated under reduced pressure. The residue was purified by silica gel flash chromatography (Eluent of 20-30% EtOAc in CH₂Cl₂) to give methyl 4-(2-(2-aminopyridin-3-yl)-5-(2-cyanophenyl)-3H-imidazo[4,5-b]pyridin-3-yl)benzoate (5.2 g, yield: 51%) as a yellow solid. MS: m/z=446.9 [M+H]⁺.

### Step 3: 2-(2-(2-Aminopyridin-3-yl)-3-(4-(hydroxymethyl)phenyl)-3H-imidazo[4,5-b]pyridin-5-yl)benzonitrile

To a solution of methyl 4-(2-(2-aminopyridin-3-yl)-5-(2-cyanophenyl)-3H-imidazo[4,5-b]pyridin-3-yl)benzoate (5 g, 11.2 mmol) in THF (50 mL) was added LiAlH₄ (2.5 M, 5.38 mL) at 0° C., then the mixture was stirred at 25° C. for 2 hr. The reaction mixture was quenched with Na₂SO₄·10H₂O (10 g) at 0° C. The mixture was filtered and the filter cake was washed by CH₂Cl₂ (30 mL×3). The filtrate was concentrated under reduced pressure to give 2-(2-(2-aminopyridin-3-yl)-3-(4-(hydroxymethyl)phenyl)-3H-imidazo[4,5-b]pyridin-5-yl)benzonitrile (4.53 g, yield: 84%) as a yellow solid, which was used to the next step without purification. MS: m/z=421.0 [M+H]⁺.

### Step 4: 2-(2-(2-Aminopyridin-3-yl)-3-(4-(chloromethyl)phenyl)-3H-imidazo[4,5-b]pyridin-5-yl)benzonitrile

To a solution of 2-(2-(2-aminopyridin-3-yl)-3-(4-(hydroxymethyl)phenyl)-3H-imidazo[4,5-b]pyridin-5-yl)benzonitrile (4.5 g, 10.8 mmol) in CH₂Cl₂ (40 mL) was added SOCl₂ (3.84 g, 32.3 mmol) at 0° C. The mixture was stirred at 40° C. for 3 hr. The reaction mixture was concentrated under reduced pressure to give 2-(2-(2-aminopyridin-3-yl)-3-(4-(chloromethyl)phenyl)-3H-imidazo[4,5-b]pyridin-5-yl)benzonitrile (Intermediate 93, 3.5 g, yield: 60%) as a yellow solid. MS: m/z=437.0

### Intermediate 94: 1-(2-(2-Aminopyridin-3-yl)-3-(4-(chloromethyl)phenyl)-3H-imidazo[4,5-b]pyridin-5-yl)-1,4-diazepan-5-one

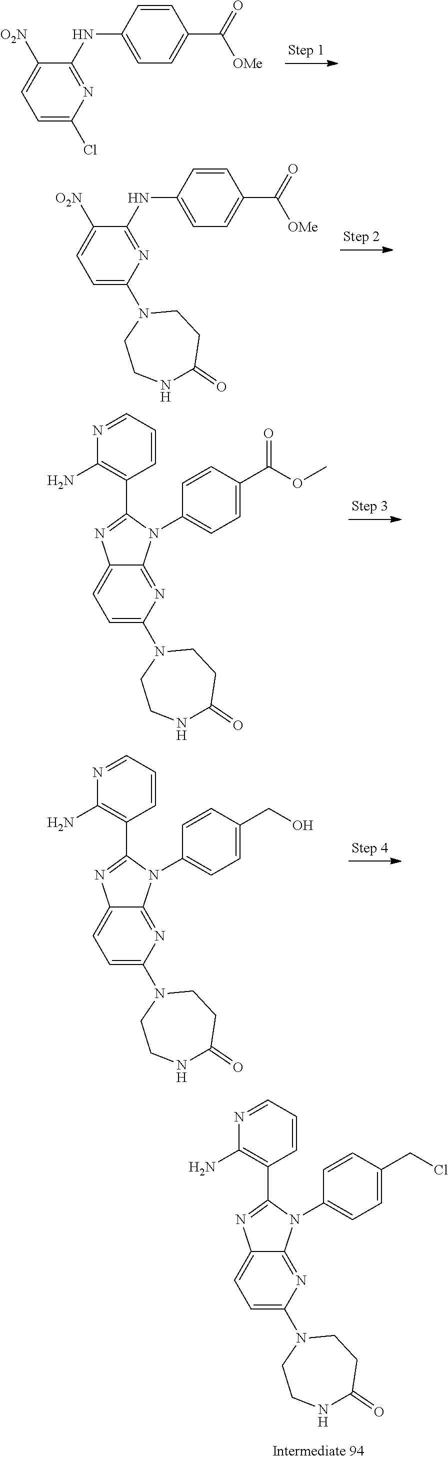

### Step 1: Methyl 4-((3-nitro-6-(5-oxo-1,4-diazepan-1-yl)pyridin-2-yl)amino)benzoate

To a solution of methyl 4-((6-chloro-3-nitropyridin-2-yl)amino)benzoate (refer to Intermediate 13 for detail procedures, 2 g, 6.50 mmol) in CH₃CN (50 mL) were added DIEA (2.52 g, 3.40 mL) and 1,4-diazepan-5-one (1.47 g, HCl salt, 9.75 mmol). The mixture was stirred at 90° C. for 12 hr. Water (10 mL) was added to the mixture and the mixture was extracted with EtOAc (20 mL×2). The combined organic layers were washed with brine (10 mL), dried over anhydrous Na₂SO₄, filtered and concentrated. Methyl 4-((3-nitro-6-(5-oxo-1,4-diazepan-1-yl)pyridin-2-yl)amino)benzoate (2.2 g, yield: 88%) was obtained as a yellow solid, which was used in the next step directly. MS: m/z=386.3 [M+H]+. ¹H NMR (400 MHz, Dimethylsulfoxide-d₆) 9.29 (br s, 1H), 8.26 (d, J=9.6 Hz, 1H), 7.97 (d, J=8.8 Hz, 2H), 7.78 (d, J=8.4 Hz, 2H), 6.59 (d, J=10.0 Hz, 1H), 3.84 (s, 3H), 3.39-3.18 (m, 4H), 3.16-3.11 (m, 2H), 2.68-2.58 (m, 2H).

### Step 2: Methyl 4-(2-(2-aminopyridin-3-yl)-5-(5-oxo-1,4-diazepan-1-yl)-3H-imidazo[4,5-b]pyridin-3-yl)benzoate

To a solution of methyl 4-((3-nitro-6-(5-oxo-1,4-diazepan-1-yl)pyridin-2-yl)amino)benzoate (2 g, 5.19 mmol) in DMSO (50 mL) were added 2-aminopyridine-3-carbaldehyde (697 mg, 5.71 mmol) and Na₂S₂O₄ (2.71 g, 15.6 mmol). The mixture was stirred at 100° C. for 12 hr. Water (100 mL) was added to the mixture. The mixture was extracted with CH₂Cl₂ (50 mL×2). The combined organic layers were washed with brine (10 mL), dried over anhydrous Na₂SO₄, filtered and concentrated to give methyl 4-(2-(2-aminopyridin-3-yl)-5-(5-oxo-1,4-diazepan-1-yl)-3H-imidazo[4,5-b]pyridin-3-yl)benzoate (2.3 g, yield: 97%) as a yellow solid. MS: m/z=458.2 [M+H]⁺.

### Step 3: 1-(2-(2-Aminopyridin-3-yl)-3-(4-(hydroxymethyl)phenyl)-3H-imidazo[4,5-b]pyridin-5-yl)-1,4-diazepan-5-one

Three-necked round 100 mL bottom flask equipped with thermometer was charged with a solution of methyl 4-(2-(2-aminopyridin-3-yl)-5-(5-oxo-1,4-diazepan-1-yl)-3H-imidazo[4,5-b]pyridin-3-yl)benzoate (500 mg, 1.09 mmol) in THF (5 mL). The flask was degassed and purged with N₂ three times, then LiAlH₄ (133 mg, 3.50 mmol) was added in portions under N₂ at 0° C. The mixture was stirred at 0° C. for 0.5 hr, then was warmed to 25° C. After stirring for 2 h under N₂, the mixture was quenched with water (10 mL), 15% NaOH (aq. 5 mL) and water (15 ml) at 0° C. The resulting mixture was stirred for 1 hr. The solid was filtered off and the filtrate was concentrated. The crude was purified by silica gel flash silica gel flash chromatography (Eluent of 0˜10% MeOH in CH₂Cl₂) to give 1-(2-(2-aminopyridin-3-yl)-3-(4-(hydroxymethyl)phenyl)-3H-imidazo[4,5-b]pyridin-5-yl)-1,4-diazepan-5-one (50 mg, yield: 11%) as yellow oil. MS: m/z=430.3 [M+H]⁺.

### Step 4: 1-(2-(2-Aminopyridin-3-yl)-3-(4-(chloromethyl)phenyl)-3H-imidazo[4,5-b]pyridin-5-yl)-1,4-diazepan-5-one

To a solution of 1-(2-(2-aminopyridin-3-yl)-3-(4-(hydroxymethyl)phenyl)-3H-imidazo[4,5-b]pyridin-5-yl)-1,4-diazepan-5-one (50 mg, 116 μmol) in CH₂Cl₂ (5 mL) was added SOCl₂ (41.6 mg, 25.4 μL). The mixture was stirred at 20° C. for 1 hr. The reaction mixture was concentrated to give 1-(2-(2-aminopyridin-3-yl)-3-(4-(chloromethyl)phenyl)-3H-imidazo[4,5-b]pyridin-5-yl)-1,4-diazepan-5-one (Intermediate 94, 52.2 mg, yield: 100′%) as yellow solid. MS: m/z=448.3 [M+H]⁺.

### Intermediate 95: 1-(2-(2-Aminopyridin-3-yl)-3-(4-(chloromethyl)phenyl)-3H-imidazo[4,5-b]pyridin-5-yl)-4-methyl-1,4-diazepan-5-one

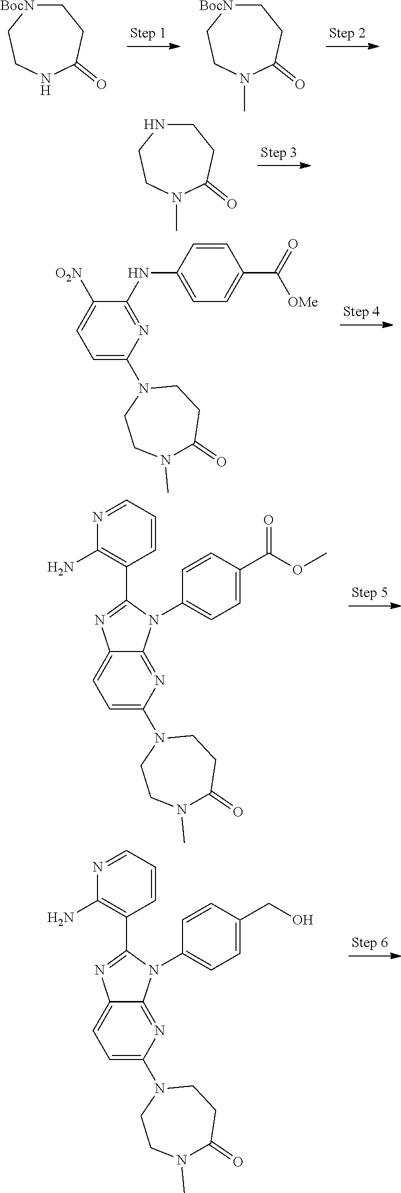

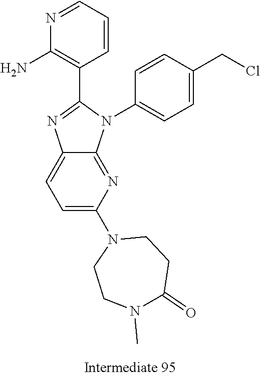 [SMILES: CN1CCN(c2ccc3nc(-c4cccnc4N)n(-c4ccc(CCl)cc4)c3n2)CCC1=O]

### Step 1: Tert-butyl 4-methyl-5-oxo-1,4-diazepane-1-carboxylate

To a solution of tert-butyl 5-oxo-1,4-diazepane-1-carboxylate (5 g, 23.3 mmol) in THF (50 mL) were added MeI (4.97 g, 35.0 mmol, 2.18 mL) and NaH (1.87 g, 46.7 mmol, 60% purity) at 0° C. under N₂. This mixture was stirred at 25° C. for 3 h. The mixture was quenched with water (10 mL) at 0° C. and then phase was extracted with CH₂Cl₂ (20 mL×3). The combined organic phase was washed with brine (30 mL), dried over anhydrous Na₂SO₄, filtered and concentrated to give Tert-butyl 4-methyl-5-oxo-1,4-diazepane-1-carboxylate (5.33 g, yield: 100%) as light-yellow oil, which was used next step without purification. ¹H NMR (400 MHz, Dimethylsulfoxide-d₆) δ 3.51-3.39 (m, 6H), 2.84 (s, 3H), 2.56-2.52 (m, 2H), 1.39 (s,

### Step 2: 4-Methyl-1,4-diazepan-5-one

To a solution of tert-butyl 4-methyl-5-oxo-1,4-diazepane-1-carboxylate (5.33 g, 23.4 mmol) in 1,4-dioxane (10 mL) was added HCl/1,4-dioxane (4 M, 40 mL) at 25° C. under N₂. This mixture was stirred at 25° C. for 3 hr. The reaction was concentrated. 4-Methyl-1,4-diazepan-5-one (3.84 g, HCl salt, yield: 100%) was obtained as a light-yellow solid, which was used next step without purification. ¹H NMR (400 MHz, Dimethylsulfoxide-d₆) δ 9.63-9.39 (m, 1H), 3.71-3.63 (m, 2H), 3.22-3.09 (m, 4H), 2.87 (s, 3H), 2.81-2.73 (m, 2H).

### Step 3: Methyl 4-((6-(4-methyl-5-oxo-1,4-diazepan-1-yl)-3-nitropyridin-2-yl)amino)benzoate

To a solution methyl 4-((6-chloro-3-nitropyridin-2-yl)amino)benzoate (refer to Intermediate 13 for detail procedures, 6.2 g, 20.2 mmol) in DMSO (50 mL) were added DIEA (7.81 g, 10.53 mL) and 4-methyl-1,4-diazepan-5-one (3.65 g, HCl salt, 22.2 mmol). The mixture was stirred at 80° C. for 12 hr. Water (50 mL) was added to the mixture and the mixture was extracted with EtOAc (50 mL×2). The combined organic layers were washed with brine (10 mL), dried over anhydrous Na₂SO₄, filtered and concentrated. Methyl 4-((6-(4-methyl-5-oxo-1,4-diazepan-1-yl)-3-nitropyridin-2-yl)amino)benzoate (8.05 g, yield: 100%) was obtained as a light-yellow solid. MS: m/z=400.2 [M+H]⁺. ¹H NMR (400 MHz, Chloroform-d) δ 10.78 (s, 1H), 8.32 (d, J=9.2 Hz, 1H), 8.03 (d, J=8.8 Hz, 2H), 7.62 (d, J=8.4 Hz, 2H), 6.21 (d, J=9.6 Hz, 1H), 3.99-3.94 (m, 2H), 3.92 (s, 3H), 3.88-3.82 (m, 2H), 3.58-3.507 (m, 2H), 3.04 (s, 3H), 2.82-2.76 (m, 2H).

### Step 4: Methyl 4-(2-(2-aminopyridin-3-yl)-5-(4-methyl-5-oxo-1,4-diazepan-1-yl)-3H-imidazo[4,5-b]pyridin-3-yl)benzoate

To a solution of methyl 4-((6-(4-methyl-5-oxo-1,4-diazepan-1-yl)-3-nitropyridin-2-yl)amino)benzoate (7.99 g, 20 mmol) in DMSO (80 mL) were added Na₂S₂O₄ (10.5 g, 60 mmol) and 2-aminopyridine-3-carbaldehyde (2.44 g, 20 mmol). The mixture was stirred at 100° C. for 12 hr. The reaction was concentrated. Water (100 mL) was added and the aqueous was extracted with CH₂Cl₂ (200 mL×2). The combined organic layers were dried over anhydrous Na₂SO₄, filtered and concentrated. Methyl 4-42-(2-aminopyridin-3-yl)-5-(4-methyl-5-oxo-1,4-diazepan-1-yl)-3H-imidazo[4,5-b]pyridin-3-yl)benzoate (7 g, yield: 74%) was obtained as a yellow solid, which was used in next step without purification. MS: m/z=472.2 [M+H]⁺. ¹H NMR (400 MHz, Dimethylsulfoxide-d₆) δ 8.09 (d, J=8.8 Hz, 2H), 8.04-7.95 (m, 2H), 7.57 (d, J=8.8 Hz, 2H), 7.30 (d, J=6.8 Hz, 1H), 6.95 (d, J=9.2 Hz, 1H), 6.57 (dd, J=7.6, 5.6 Hz, 1H), 3.89 (s, 3H), 3.80-3.74 (m, 2H), 3.73-3.68 (m, 2H), 3.54-3.44 (m, 2H), 2.82 (s, 3H), 2.64-2.60 (m, 2H).

### Step 5: 1-(2-(2-Aminopyridin-3-yl)-3-(4-(hydroxymethyl)phenyl)-3H-imidazo[4,5-b]pyridin-5-yl)-4-methyl-1,4-diazepan-5-one

To a solution of methyl methyl 4-(2-(2-aminopyridin-3-yl)-5-(4-methyl-5-oxo-1,4-diazepan-1-yl)-3H-imidazo[4,5-b]pyridin-3-yl)benzoate (100 mg, 212 μmol) in THF (1 mL) was added LiAlH₄ (24.2 mg, 636 μmol) at 0° C. for 30 min. The mixture was stirred at 0° C. for 1.5 hr. The mixture was diluted with THF (20 mL). Na₂SO₄·10H₂O was added in portions until no bubbles were formed. The resulting mixture was stirred at 25° C. for 20 min and filtered. The filter cake was washed with THF (20 mL×2), the combined filtrate was concentrated. 1-(2-(2-Aminopyridin-3-yl)-3-(4-(hydroxymethyl)phenyl)-3H-imidazo[4,5-b]pyridin-5-yl)-4-methyl-1,4-diazepan-5-one (82 mg, yield: 87%) was obtained as light-yellow oil, which was used next step without further purification. MS: m/z=444.1 [M+H]⁺.

### Step 6: 1-(2-(2-Aminopyridin-3-yl)-3-(4-(chloromethyl)phenyl)-3H-imidazo[4,5-b]pyridin-5-yl)-4-methyl-1,4-diazepan-5-one

To a solution of 1-(2-(2-aminopyridin-3-yl)-3-(4-(hydroxymethyl)phenyl)-3H-imidazo[4,5-b]pyridin-5-yl)-4-methyl-1,4-diazepan-5-one (82 mg, 185 μmol) in CH₂Cl₂ (1 mL) were added SOCl₂ (110 mg, 924 μmol). The mixture was stirred at 25° C. for 12 hr. The reaction was concentrated. 1-(2-(2-Aminopyridin-3-yl)-3-(4-(chloromethyl)phenyl)-3H-imidazo[4,5-b]pyridin-5-yl)-4-methyl-1,4-diazepan-5-one (Intermediate 95, 85.4 mg, yield: 100%) was obtained as a light-yellow solid, which was used for next step without purification. MS: m/z=462.1 [M+H]+.

### Intermediate 96: 3-(5-(Cyclohex-1-en-1-yl)-3-(4-(piperazin-1-ylmethyl)phenyl)-3H-imidazo[4,5-b]pyridin-2-yl)pyridin-2-amine

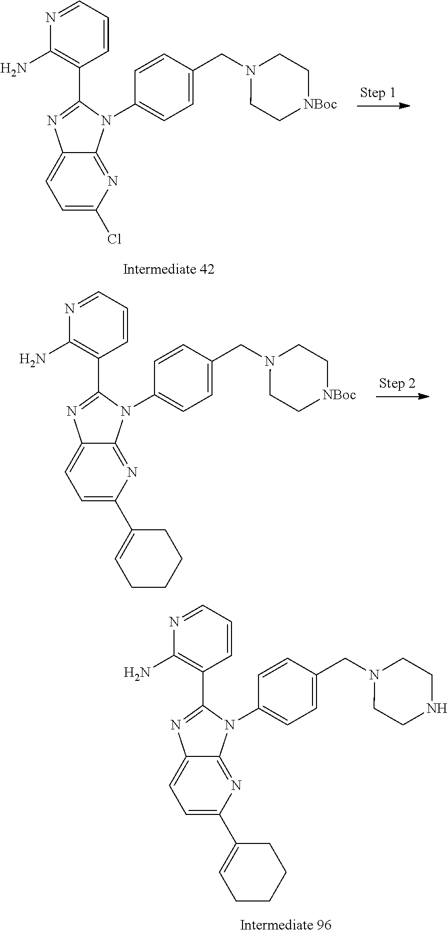

### Step 1: Tert-butyl 4-(4-(2-(2-aminopyridin-3-yl)-5-(cyclohex-1-en-1-yl)-3H-imidazo[4,5-b]pyridin-3-yl)benzyl)piperazine-1-carboxylate

To a solution of Intermediate 42 (200 mg, 385 μmol) in 1,4-dioxane (5 mL) and H₂O (1 mL) were added cyclohex-1-en-1-ylboronic acid (72.7 mg, 577 μmol), K₂CO₃ (159 mg, 1.15 mmol) and Pd(dppf)Cl₂ (28.1 mg, 38.5 μmol) at 25° C. under N₂. This mixture was stirred at 100° C. for 5 hr. H₂O (5 mL) was added to the mixture. The aqueous phase was extracted with EtOAc (20 mL×3). The combined organic layers were washed with brine (30 mL), dried over anhydrous Na₂SO₄, filtered and concentrated. The residue was purified by prep-TLC (EtOAc: petroleum ether=2:1) to give tert-butyl 4-(4-(2-(2-aminopyridin-3-yl)-5-(cyclohex-1-en-1-yl)-3H-imidazo[4,5-b]pyridin-3-yl)benzyl)piperazine-1-carboxylate (100 mg, yield: 83%) as a yellow oil. MS: m/z=566.4 [M+H]⁺.

### Step 2: 3-(5-(Cyclohex-1-en-1-yl)-3-(4-(piperazin-1-ylmethyl)phenyl)-3H-imidazo[4,5-b]pyridin-2-yl)pyridin-2-amine

To a solution of tert-butyl 4-(4-(2-(2-aminopyridin-3-yl)-5-(cyclohex-1-en-1-yl)-3H-imidazo[4,5-b]pyridin-3-yl)benzyl)piperazine-1-carboxylate (79.7 mg, 141 μmol) in 1,4-dioxane (1 mL) was added HCl/1,4-dioxane (4 M, 35.2 μL) at 25° C. This mixture was stirred at 25° C. for 3 hr. The mixture was filtrated and concentrated. 3-(5-(Cyclohex-1-en-1-yl)-3-(4-(piperazin-1-ylmethyl)phenyl)-3H-imidazo[4,5-b]pyridin-2-yl)pyridin-2-amine (Intermediate 96, 60 mg, HCl salt, yield: 84%) was obtained as a yellow solid, which was used in the next step directly without purification. MS: m/z=466.2 [M+H]+. ¹H NMR (400 MHz, Methanol-d₄) δ 8.16 (d, J=8.4 Hz, 1H), 8.01 (dd, J=6.4, 1.6 Hz, 1H), 7.90 (d, J=8.4 Hz, 2H), 7.85 (dd, J=7.6, 1.4 Hz, 1H), 7.70-7.62 (m, 3H), 6.92 (dd, J=7.6, 6.4 Hz, 1H), 6.75-6.69 (m, 1H), 4.62 (s, 2H), 3.69-3.68 (m, 7H), 3.60-3.59 (m, 1H), 2.56-2.47 (m, 2H), 2.30-2.21 (m, 2H), 1.81-1.64 (m, 4H).

### Intermediate 97: 3-(5-(3,6-Dihydro-2H-pyran-4-yl)-3-(4-(piperazin-1-ylmeth phenyl)-3H-imidazo[4,5-b]pyridin-2-yl)pyridin-2-amine

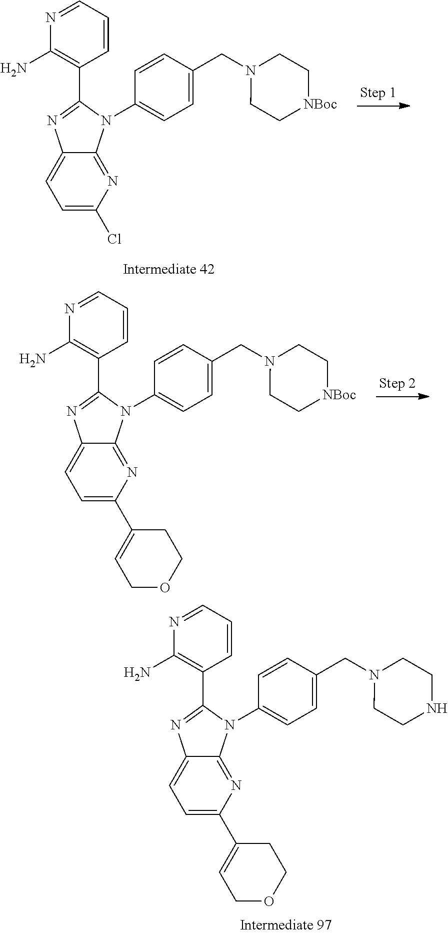

### Step 1: Tert-butyl 4-(4-(2-(2-aminopyridin-3-yl)-5-(3,6-dihydro-2H-pyran-4-yl)-3H-imidazo[4,5-b]pyridin-3-yl)benzyl)piperazine-1-carboxylate

To a solution of Intermediate 42 (200 mg, 385 μmol) in 1,4-dioxane (5 mL) and H₂O (1 mL) were added 2-(3,6-dihydro-2H-pyran-4-yl)-4,4,5,5-tetramethyl-1,3,2-dioxaborolane (121 mg, 577 μmol), K₂CO₃ (159 mg, 1.15 mmol) and Pd(dppf)Cl₂ (28.1 mg, 38.5 μmol) at 25° C. under N₂. This mixture was stirred at 100° C. for 5 hr. The water (5 mL) was added to the mixture. The aqueous phase was extracted with EtOAc (20 mL×3). The combined organic layers were washed with brine (30 mL), dried over anhydrous Na₂SO₄, filtered and concentrated. The residue was purified by prep-TLC (EtOAc: petroleum ether=2:1) to give tert-butyl 4-(4-(2-(2-aminopyridin-3-yl)-5-(3,6-dihydro-2H-pyran-4-yl)-3H-imidazo[4,5-b]pyridin-3-yl)benzyl)piperazine-1-carboxylate (45 mg, yield: 50%) as an off-white solid. MS: m/z=568.3 [M+H]⁺. ¹H NMR (400 MHz, Methanol-d₄) δ 8.08 (d, J=8.4 Hz, 1H), 7.97 (dd, J=4.8, 1.6 Hz, 1H), 7.62 (d, J=8.4 Hz, 1H), 7.51 (d, J=8.0 Hz, 2H), 7.39 (d, J=8.4 Hz, 2H), 7.29 (dd, J=8.0, 2.0 Hz, 1H), 6.72-6.65 (m, 1H), 6.47 (dd, J=8.0, 5.2 Hz, 1H), 4.36-4.28 (m, 2H), 3.90 (t, J=5.6 Hz, 2H), 3.62 (s, 2H), 3.46-3.45 (m, 4H), 2.67-2.60 (m, 2H), 2.46 (t, J=4.8 Hz, 4H), 1.46 (s, 9H).

### Step 2: 3-(5-(3,6-Dihydro-2H-pyran-4-yl)-3-(4-(piperazin-1-ylmethyl)phenyl)-3H-imidazo[4,5-b]pyridin-2-yl)pyridin-2-amine

To a solution of tert-butyl 4-(4-(2-(2-aminopyridin-3-yl)-5-(3,6-dihydro-2H-pyran-4-yl)-3H-imidazo[4,5-b]pyridin-3-yl)benzyl)piperazine-1-carboxylate (45.0 mg, 79.3 μmol) in 1,4-dioxane (1 mL) was added HCl/1,4-dioxane (4 M, 19.8 μL) at 25° C. This mixture was stirred at 25° C. for 3 hr. The mixture was filtrated and concentrated. 3-(5-(3,6-Dihydro-2H-pyran-4-yl)-3-(4-(piperazin-1-ylmethyl)phenyl)-3H-imidazo[4,5-b]pyridin-2-yl)pyridin-2-amine (Intermediate 97, 20.8 mg, HCl salt, yield: 52%) was obtained as a yellow solid, which was used in the next step directly without purification. MS: m/z=468.2 [M+H]⁺. ¹H NMR (400 MHz, Methanol-d₄) δ 8.21 (d, J=8.4 Hz, 1H), 8.01 (dd, J=6.4, 1.2 Hz, 1H), 7.93-7.83 (m, 3H), 7.75-7.62 (m, 3H), 6.96-6.87 (m, 1H), 6.75-6.74 (m, 1H), 4.62 (s, 2H), 4.39-4.29 (m, 2H), 3.90 (t, J=5.2 Hz, 2H), 3.69-3.68 (m, 8H), 2.68-2.57 (m, 2H).

### Intermediate 98: 3-(2-(2-Aminopyridin-3-yl)-3-(4-(piperazin-1-ylmethyl)phenyl)-3H-imidazo[4,5-b]pyridin-5-yl)benzonitrile

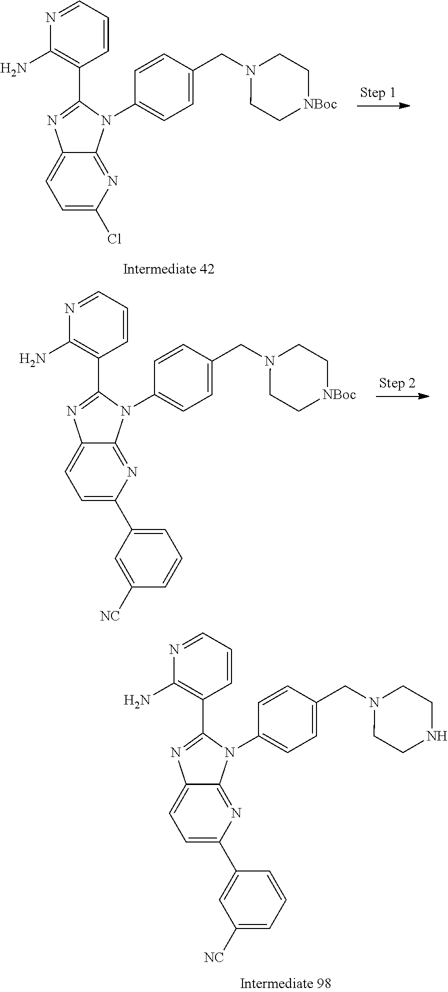 [SMILES: CC(C)(C)OC(=O)N1CCN(Cc2ccc(-n3c(-c4cccnc4N)nc4ccc(Cl)nc43)cc2)CC1 ; CC(C)(C)OC(=O)N1CCN(Cc2ccc(-n3c(-c4cccnc4N)nc4ccc(-c5cccc(C#N)c5)nc43)cc2)CC1 ; N#Cc1cccc(-c2ccc3nc(-c4cccnc4N)n(-c4ccc(CN5CCNCC5)cc4)c3n2)c1]

### Step 1: Tert-butyl 4-(4-(2-(2-aminopyridin-3-yl)-5-(3-cyanophenyl)-3H-imidazo[4,5-b]pyridin-3-yl)benzyl)piperazine-1-carboxylate

A mixture of Intermediate 42 (220 mg, 423 μmol), (3-cyanophenyl)boronic acid (68.4 mg, 465 μmol), Cs₂CO₃ (414 mg, 1.27 mmol), Pd(dppf)Cl₂ (155 mg, 212 μmol) in 1,4-dioxane (5 mL) and H₂O (1 mL) was degassed and purged with N₂ three times, and then the mixture was stirred at 100° C. for 16 hr under N₂ atmosphere. The reaction mixture was filtered at 25° C. Then it was diluted with H₂O (20 mL) and extracted with CH₂Cl₂ (20 mL×3). The combined organic layers were washed with brine (30 mL), dried over Na₂SO₄, filtered and concentrated under reduced pressure. After purified by silica gel flash chromatography (Eluent of 0˜30% EtOAc in petroleum ether), tert-butyl 4-(4-(2-(2-aminopyridin-3-yl)-5-(3-cyanophenyl)-3H-imidazo[4,5-b]pyridin-3-yl)benzyl)piperazine-1-carboxylate (240 mg, yield: 97%) was obtained as a brown solid. MS: m/z=587.4 [M+H]⁺.

### Step 2: 3-(2-(2-Aminopyridin-3-yl)-3-(4-(piperazin-1-ylmethyl)phenyl)-3H-imidazo[4,5-b]pyridin-5-yl)benzonitrile

To a solution of tert-butyl 4-(4-(2-(2-aminopyridin-3-yl)-5-(3-cyanophenyl)-3H-imidazo[4,5-b]pyridin-3-yl)benzyl)piperazine-1-carboxylate (240 mg, 409 μmol) in 1,4-dioxane (3 mL) was added 4 M HCl in 1,4-dioxane (3 mL) at 25° C. The mixture was stirred at 25° C. for 16 hr. The reaction mixture was filtered and the collected solid was washed with 1,4-dioxane (2 mL×2) and dried in vacuo to give 3-(2-(2-aminopyridin-3-yl)-3-(4-(piperazin-1-ylmethyl)phenyl)-3H-imidazo[4,5-b]pyridin-5-yl)benzonitrile (Intermediate 98, 203 mg HCl salt, yield: 95%) as a yellow solid. MS: m/z=487.2 [M+H]⁺. ¹H NMR (400 MHz, Methanol-d₄) δ 8.43-8.34 (m, 3H), 8.11 (d, J=8.4 Hz, 1H), 8.03 (d, J=5.6 Hz, 1H), 7.96 (d, J=8.4 Hz, 2H), 7.90 (d, J=7.6 Hz, 1H), 7.79-7.73 (m, 3H), 7.68-7.63 (m, 1H), 6.96-6.90 (m, 1H), 3.70-3.68 (m, 6H), 3.67-3.65 (m, 4H).

### Intermediate 99: 5-(2-(2-aminopyridin-3-yl)-3-(4-(piperazin-1-ylmethyl)phenyl)-3H-imidazo[4,5-b]pyridin-5-yl)pyridin-2(11H)-one

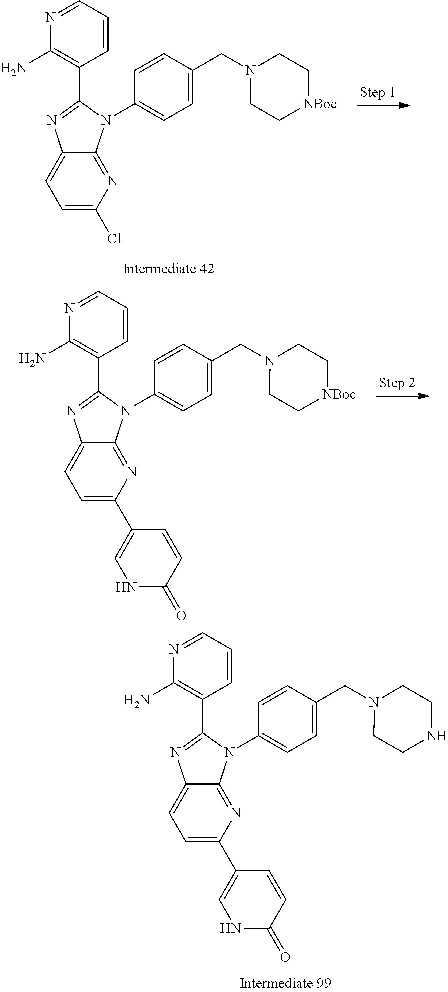

### Step 1: Tert-butyl 4-(4-(2-(2-aminopyridin-3-yl)-5-(6-oxo-1,6-dihydropyridin-3-yl)-3H-imidazo[4,5-b]pyridin-3-yl)benzyl)piperazine-1-carboxylate

A mixture of Intermediate 42 (200 mg, 385 μmol), (6-oxo-1,6-dihydropyridin-3-yl)boronic acid (54 mg, 385 μmol), Cs₂CO₃ (376 mg, 1.15 mmol), Pd(dppf)Cl₂ (31.4 mg, 38.5 μmol) in 1,4-dioxane (2.5 mL) and H₂O (0.5 mL) was degassed and purged with N₂ three times, and then the mixture was stirred at 100° C. for 16 hr under N₂ atmosphere. The reaction mixture was filtered at 25° C., and then diluted with H₂O (10 mL) and extracted with CH₂Cl₂ (10 mL×3). The combined organic layers were washed with brine (30 mL), dried over Na₂SO₄, filtered and concentrated under reduced pressure. After purified by silica gel flash chromatography (Eluent of 0˜10% CH₂Cl₂ in MeOH), tert-butyl 4-(4-(2-(2-aminopyridin-3-yl)-5-(6-oxo-1,6-dihydropyridin-3-yl)-3H-imidazo[4,5-b]pyridin-3-yl)benzyl)piperazine-1-carboxylate (60 mg, yield: 22%) was obtained as a yellow solid. MS: m/z=579.2 [M+H]⁺.

### Step 2: 5-(2-(2-Aminopyridin-3-yl)-3-(4-(piperazin-1-ylmethyl)phenyl)-3H-imidazo[4,5-b]pyridin-5-yl)pyridin-2(1H)-one

To a solution of tert-butyl 4-(4-(2-(2-aminopyridin-3-yl)-5-(6-oxo-1,6-dihydropyridin-3-yl)-3H-imidazo[4,5-b]pyridin-3-yl)benzyl)piperazine-1-carboxylate (60 mg, 104 μmol) in 1,4-dioxane (2 mL) was added 4 M HCl in 1,4-dioxane (1 mL) at 25° C. The mixture was stirred at 25° C. for 16 hr. The reaction mixture was then filtered and the collected solid was washed with 1,4-dioxane (5 mL×2) and dried in vacuo to give 5-(2-(2-aminopyridin-3-yl)-3-(4-(piperazin-1-ylmethyl)phenyl)-3H-imidazo[4,5-b]pyridin-5-yl)pyridin-2(1H)-one (Intermediate 99, 48.6 mg, yield: 89/a) as a yellow solid. MS: m/z=479.2 [M+H]⁺. ¹H NMR (400 MHz, Methanol-d₄) δ 8.76 (d, J=9.2 Hz, 1H), 8.61 (s, 1H), 8.37 (d, J=8.4 Hz, 1H), 8.01-8.07 (m, 2H), 7.95 (d, J=8.0 Hz, 2H), 7.90 (d, J=7.2 Hz, 1H), 7.73 (d, J=7.6 Hz, 2H), 7.08 (d, J=9.2 Hz, 1H), 6.94 (t, J=6.8 Hz, 1H), 4.67 (s, 2H), 3.82-3.64 (m, 8H).

### Intermediate 100: 2-(3-(4-((4-Aminopiperidin-1-yl)methyl)phenyl)-2-(2-aminopyridin-3-yl)-3H-imidazo[4,5-b]pyridin-5-yl)benzonitrile

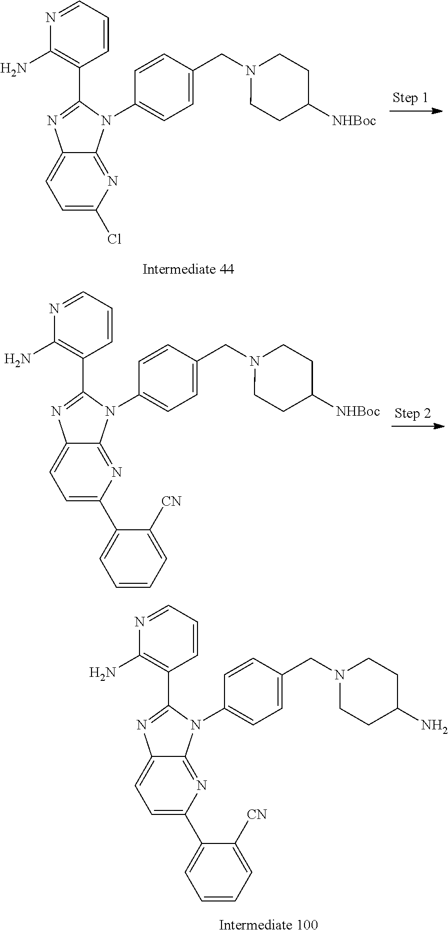

### Step 1: Tert-butyl (1-(4-(2-(2-aminopyridin-3-yl)-5-(2-cyanophenyl)-3H-imidazo[4,5-b]pyridin-3-yl)benzyl)piperidin-4-yl)carbamate

A mixture of Intermediate 44 (100 mg, 187 μmol), (2-cyanophenyl)boronic acid (55 mg, 375 μmol) in DMF (2 mL), cataCXiumAPdG3 (68 mg, 94 μmol), K₃PO₄ (80 mg, 375 μmol), PCy₃ (5 mg, 19 μmol) in 1,4-dioxane (2.5 mL) and H₂O (0.5 mL) was degassed and purged with N₂ three times, and then the mixture was stirred at 120° C. for 16 hr under N₂ atmosphere. The reaction mixture was poured into H₂O (15 mL), extracted with EtOAc (60 mL×2). The combined organic layers were dried over anhydrous Na₂SO₄, filtered and concentrated under reduced pressure. The residue was purified by silica gel flash chromatography (Eluent of 0˜6% MeOH in CH₂Cl₂) to give tert-butyl (1-(4-(2-(2-aminopyridin-3-yl)-5-(2-cyanophenyl)-3H-imidazo[4,5-b]pyridin-3-yl)benzyl)piperidin-4-yl)carbamate (76 mg, yield: 68%) as a yellow solid. MS: m/z=601.2 [M+H]⁺.

### Step 2: 2-(3-(4-((4-Aminopiperidin-1-yl)methyl)phenyl)-2-(2-aminopyridin-3-yl)-3H-imidazo[4,5-b]pyridin-5-yl)benzonitrile

To a solution of tert-butyl (1-(4-(2-(2-aminopyridin-3-yl)-5-(2-cyanophenyl)-3H-imidazo[4,5-b]pyridin-3-yl)benzyl)piperidin-4-yl)carbamate (152 mg, 253 μmol) in CH₂Cl₂ (2 mL) was added 4 M HCl in 1,4-dioxane (2.0 mL). The mixture was stirred at 25° C. for 1 hr. The reaction mixture was concentrated. The crude was triturated with CH₂Cl₂ at 25° C. for 30 min. 2-(3-(4-((4-Aminopiperidin-1-yl)methyl)phenyl)-2-(2-aminopyridin-3-yl)-3H-imidazo[4,5-b]pyridin-5-yl)benzonitrile (Intermediate 100, 114 mg, HCl salt, yield: 84%) was obtained as a yellow solid. MS: m/z=501.2 [M+H]⁺. ¹H NMR (400 MHz, Methanol-d₄) δ 8.42 (d, J=8.0 Hz, 1H), 8.06-8.01 (m, 1H), 7.96-7.90 (m, 31H), 7.85-7.83 (m, 3H), 7.80-7.75 (m, 3H), 7.62-7.55 (m, 1H), 6.88 (t, J=7.2 Hz, 1H), 3.69-3.65 (m, 2H), 3.62-3.35 (m, 2H), 3.29-3.21 (m, 3H), 2.30-2.26 (m, 2H), 2.12-2.01 (m, 2H).

### Intermediate 101: 3-(3-(4-(((3aR,6aS)-hexahydropyrrolo[3,4-c]pyrrol-2(1H)-yl)methyl)phenyl)-5-phenyl-3H-imidazo[4,5-b]pyridin-2-yl)pyridin-2-amine

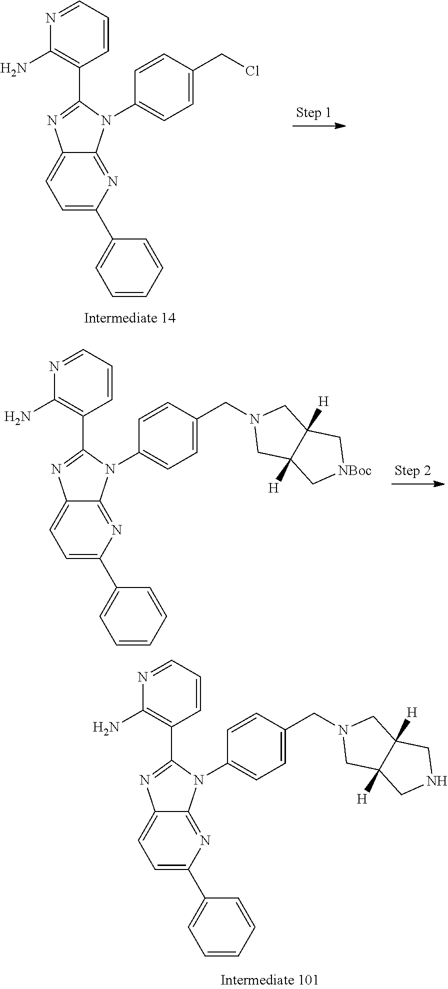

### Step 1: Tert-butyl (3aR,6aS)-5-(4-(2-(2-aminopyridin-3-yl)-5-phenyl-3H-imidazo[4,5-b]pyridin-3-yl)benzyl)hexahydropyrrolo[3,4-c]pyrrole-2(1H)-carboxylate

To a solution of Intermediate 14 (4.6 g, 11.2 mmol) and tert-butyl (3aR,6aS)-2,3,3a,4,6,6a-hexahydro-1H-pyrrolo[3,4-c]pyrrole-5-carboxylate (2.61 g, 12.3 mmol) in DMF (60 mL) were added NaI (502 mg, 3.35 mmol) and K₂CO₃ (3.09 g, 22.3 mmol) in one portion at 25° C. Then the mixture was stirred at 80° C. for 12 hr. The reaction mixture was poured into water (100 mL) at 25° C., then extracted with EtOAc (100 mL×3). The combined organic layers were washed with brine (150 mL), dried over anhydrous Na₂SO₄, filtered and concentrated under reduced pressure. The residue was purified by silica gel flash chromatography (Eluent of 0˜6% MeOH in CH₂Cl₂) to give tert-butyl (3aR,6aS)-5-(4-(2-(2-aminopyridin-3-yl)-5-phenyl-3H-imidazo[4,5-b]pyridin-3-yl)benzyl)hexahydropyrrolo[3,4-c]pyrrole-2(11H)-carboxylate (3.6 g, yield: 55%) as a yellow solid. MS: m/z=588.4 [M+H]⁺. ¹H NMR (400 MHz, Dimethylsulfoxide-d₆) δ 8.27 (d, J=8.4 Hz, 1H), 8.06-7.96 (m, 4H), 7.50-7.36 (m, 7H), 7.16 (dd, J=7.6, 1.6 Hz, 1H), 7.03 (br s, 2H), 6.38 (dd, 0.1=7.6, 4.8 Hz, 1H), 3.65 (s, 2H), 3.51-3.41 (m, 2H), 3.17-3.10 (m, 2H), 2.76-2.75 (m, 2H), 2.59-2.53 (m, 2H), 2.45-2.39 (m, 2H), 1.39 (s, 9H).

### Step 2: 3-(3-(4-(((3aR,6aS)-hexahydropyrrolo[3,4-c]pyrrol-2(1H)-yl)methyl)phenyl)-5-phenyl-3H-imidazo[4,5-b]pyridin-2-yl)pyridin-2-amine

To a solution of tert-butyl (3aR,6aS)-5-(4-(2-(2-aminopyridin-3-yl)-5-phenyl-3H-imidazo[4,5-b]pyridin-3-yl)benzyl)hexahydropyrrolo[3,4-c]pyrrole-2(1H)-carboxylate (50 mg, 85.1 μmol) in 1,4-dioxane (2 mL) was added HCl/1,4-dioxane (4 M, 212 μL). The reaction mixture was stirred at 20° C. for 12 hr. The mixture was quenched with sat. NaHCO₃ (10 mL) and extracted with CH₂Cl₂ (10 mL×2). The combined organic layers were washed with brine (10 mL), dried over anhydrous Na₂SO₄, filtered and concentrated. 3-(3-(4-(((3aR,6aS)-Hexahydropyrrolo[3,4-c]pyrrol-2(1H)-yl)methyl)phenyl)-5-phenyl-3H-imidazo[4,5-b]pyridin-2-yl)pyridin-2-amine (Intermediate 101, 35.4 mg, yield: 85%) was obtained as an off-white solid. MS: m/z=488.2 [M+H]⁺. ¹H NMR (400 MHz, Dimethylsulfoxide-d₆) 8.27 (d, J=8.0 Hz, 1H), 8.04-7.97 (m, 4H), 7.49-7.44 (m, 5H), 7.43-7.37 (m, 2H), 7.16 (dd, J=7.6, 1.6 Hz, 1H), 7.04 (br s, 2H), 6.38 (dd, J=7.6, 4.8 Hz, 1H), 3.60 (s, 2H), 2.85-2.76 (m, 2H), 2.58-2.51 (m, 7H), 2.31-2.26 (m, 2H).

### Intermediate 102: 3-(3-(4-(((3aR,6aR)-hexahydropyrrolo[3,4-c]pyrrol-2(1H)-yl))methyl)phenyl)-5-phenyl-3H-imidazo[4,5-b]pyridin-2-yl)pyridin-2-amine

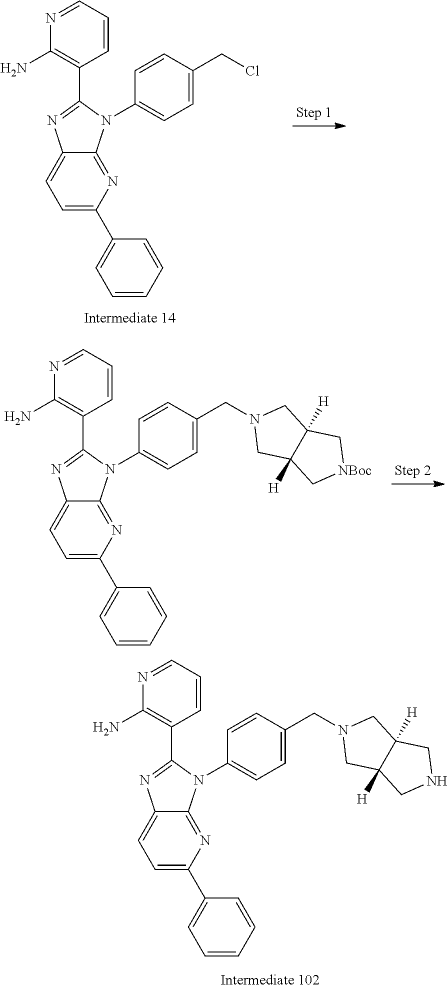

### Step 1: Tert-butyl (3aS,6aS)-5-(4-(2-(2-aminopyridin-3-yl)-5-phenyl-3H-imidazo[4,5-b]pyridin-3-yl)benzyl)hexahydropyrrolo[3,4-c]pyrrole-2(1H)-carboxylate

To a solution of Intermediate 14 (200 mg, 486 μmol) in DMF (4 mL) were added K₂CO₃ (201 mg, 1.46 mmol), NaI (21.8 mg, 146 μmol) and tert-butyl (3aS,6aS)-hexahydropyrrolo[3,4-c]pyrrole-2(1H)-carboxylate (134 mg, 631 μmol). The mixture was stirred at 25° C. for 16 hr. The reaction mixture was quenched with H₂O (10 mL) at 25° C., and then extracted with EtOAc (20 mL×3). The combined organic layers were washed with brine (20 mL), dried over Na₂SO₄, filtered and concentrated under reduced pressure. After purified by prep-TLC (CH₂Cl₂:MeOH=10:1), tert-butyl (3aS,6aS)-5-(4-(2-(2-aminopyridin-3-yl)-5-phenyl-3H-imidazo[4,5-b]pyridin-3-yl)benzyl)hexahydropyrrolo[3,4-c]pyrrole-2(1H)-carboxylate (100 mg, yield: 32%) was obtained as a yellow solid. MS: m/z=588.3 [M+H]⁺.

### Step 2: 3-(3-(4-(((3aR,6aR)-Hexahydropyrrolo[3,4-c]pyrrol-2(1H)-yl)methyl)phenyl)-5-phenyl-3H-imidazo[4,5-b]pyridin-2-yl)pyridin-2-amine

To a solution of tert-butyl (3aS,6aS)-5-(4-(2-(2-aminopyridin-3-yl)-5-phenyl-3H-imidazo[4,5-b]pyridin-3-yl)benzyl)hexahydropyrrolo[3,4-c]pyrrole-2(1H)-carboxylate (100 mg, 170 μmol) in 1,4-dioxane (2 mL) was added 4 M HCl in 1,4-dioxane (2 mL). The mixture was stirred at 25° C. for 1 hr. The reaction was concentrated under reduced pressure to give 3-(3-(4-(((3aR,6aR)-hexahydropyrrolo[3,4-c]pyrrol-2(1H)-yl)methyl)phenyl)-5-phenyl-3H-imidazo[4,5-b]pyridin-2-yl)pyridin-2-amine (Intermediate 102, 30 mg, HCl salt, yield: 33%) as a yellow solid. MS: m/z=488.3 [M+H]⁺. ¹H NMR (400 MHz, Dimethylsulfoxide-d₆) δ 12.14-11.98 (m, 1H), 9.92-9.78 (m, 1H), 9.73-9.54 (m, 1H), 8.37 (d, J=8.8 Hz, 1H), 8.33-8.19 (m, 1H), 8.13 (dd, J=6.0, 1.2 Hz, 1H), 8.09-8.04 (m, 3H), 7.86 (d, J=8.4 Hz, 2H), 7.81-7.76 (m, 1H), 7.66 (d, J=8.0 Hz, 2H), 7.51-7.41 (m, 3H), 6.89-6.83 (m, 1H), 4.66-4.51 (m, 2H), 3.72-3.63 (m, 2H), 3.20-3.10 (m, 2H), 3.04-2.93 (m, 2H), 2.85-2.76 (m, 1H), 2.64-2.57 (m, 1H), 2.46-2.33 (m, 2H).

### Intermediate 103: 4-((3aR,6aR)-Hexahydropyrrolo[3,4-c]pyrrol-2(1H)-yl)pyrimidine-2-carbonitrile

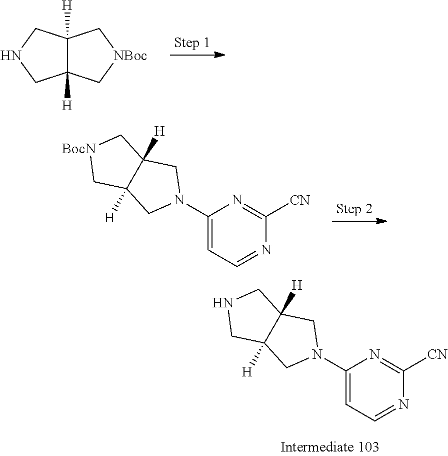

### Step 1: Tert-butyl (3aS,6aS)-5-(2-cyanopyrimidin-4-yl)hexahydropyrrolo[3,4-c]pyrrole-2(1H)-carboxylate

To a mixture of tert-butyl (3aS,6aS)-hexahydropyrrolo[3,4-c]pyrrole-2(1H)-carboxylate (300 mg, 1.41 mmol) and 4-chloropyrimidine-2-carbonitrile (197 mg, 1.41 mmol) in NMP (4 mL) was added DIEA (547 mg, 4.24 mmol, 738 μL). The mixture was stirred at 60° C. for 1 hr. H₂O (10 mL) was added and the reaction mixture was filtrated. The filtration cake was dried to give tert-butyl (3aS,6aS)-5-(2-cyanopyrimidin-4-yl)hexahydropyrrolo[3,4-c]pyrrole-2(1H)-carboxylate (230 mg, yield: 52%) as a white solid. MS: m/z=316.0 [M+H]⁺. ¹H NMR (400 MHz, Dimethylsulfoxide-d₆) 8.24 (d, J=6.4 Hz, 1H), 6.76 (d, J=6.4 Hz, 1H), 3.91-3.80 (m, 1H), 3.70-3.60 (m, 1H), 3.57-3.49 (m, 2H), 3.18-3.10 (m, 2H), 3.08-2.96 (m, 2H), 2.43-2.22 (m, 2H), 1.41 (s, 9H).

### Step 2: 4-((3aR,6aR)-Hexahydropyrrolo[3,4-c]pyrrol-2(1H)-yl)pyrimidine-2-carbonitrile

To a mixture of tert-butyl (3aS,6aS)-5-(2-cyanopyrimidin-4-yl)hexahydropyrrolo[3,4-c]pyrrole-2(1H)-carboxylate (100 mg, 317 μmol) in CH₂Cl₂ (3 mL) was added TFA (767 mg, 0.5 mL), the mixture was stirred at 20° C. for 1 hr. The mixture was concentrated to give 4-((3aR,6aR)-hexahydropyrrolo[3,4-c]pyrrol-2(1H)-yl)pyrimidine-2-carbonitrile (Intermediate 103, 104 mg, TFA salt, yield: 100%) as yellow oil. MS: m/z=215.8 [M+H]⁺.

### Intermediate 105: 5-(2-(2-Aminopyridin-3-yl)-3-(4-(chloromethyl)phenyl)-3H-imidazo[4,5-b]pyridin-5-yl)-1-methylpyridin-2(1H)-one

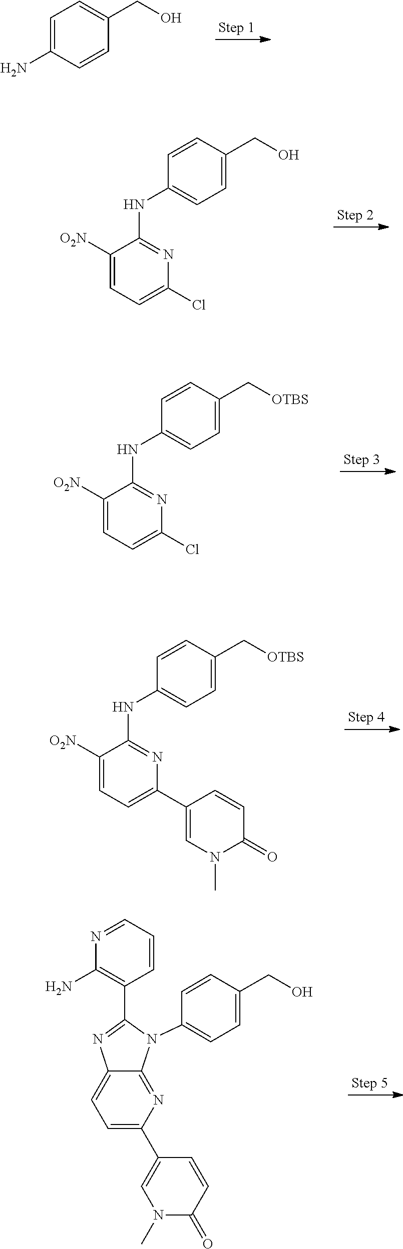 [SMILES: Nc1ccc(CO)cc1 ; O=[N+]([O-])c1ccc(Cl)nc1Nc1ccc(CO)cc1 ; CC(C)(C)[Si](C)(C)OCc1ccc(Nc2nc(Cl)ccc2[N+](=O)[O-])cc1 ; Cn1cc(-c2ccc([N+](=O)[O-])c(Nc3ccc(CO[Si](C)(C)C(C)(C)C)cc3)n2)ccc1=O ; Cn1cc(-c2ccc3nc(-c4cccnc4N)n(-c4ccc(CO)cc4)c3n2)ccc1=O]

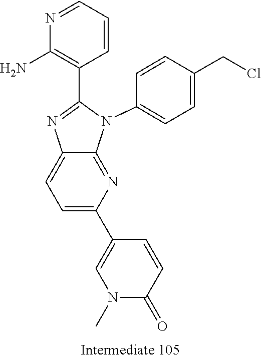 [SMILES: Cn1cc(-c2ccc3nc(-c4cccnc4N)n(-c4ccc(CCl)cc4)c3n2)ccc1=O]

### Step 1: (4-((6-Chloro-3-nitropyridin-2-yl)amino)phenyl)methanol

To a mixture of (4-aminophenyl)methanol (5 g, 40.6 mmol) and 2,6-dichloro-3-nitro-pyridine (7.84 g, 40.6 mmol) in THF (70 mL) was added DIEA (10.5 g, 81.2 mmol, 14.1 mL). The mixture was stirred at 80° C. for 1 hr. The mixture was concentrated to give (4-((6-chloro-3-nitropyridin-2-yl)amino)phenyl)methanol (11.36 g, yield: 100%) as a yellow oil. MS: m/z=279.8 [M+H]⁺.

### Step 2: N-(4-(((tert-butyldimethylsilyl)oxy)methyl)phenyl)-6-chloro-3-nitropyridin-2-amine

To a mixture of (4-((6-chloro-3-nitropyridin-2-yl)amino)phenyl)methanol (1 g, 3.58 mmol) in CH₂Cl₂ (10 mL) were added imidazole (365 mg, 5.36 mmol) and TBSCl (701 mg, 4.65 mmol). The mixture was stirred at 20° C. for 1 hr. The mixture was diluted with H₂O (50 mL) and extracted with CH₂Cl₂ (50 mL×2). The combined organic layers were washed with brine (20 mL), dried over Na₂SO₄, filtered and concentrated to give N-(4-(((tert-butyldimethylsilyl)oxy)methyl)phenyl)-6-chloro-3-nitropyridin-2-amine (1.1 g, yield: 78%) as a yellow solid. MS: m/z=394.0 [M+H]⁺. ¹H NMR (400 MHz, Dimethylsulfoxide-d₆) δ 10.12 (s, 1H), 8.54 (d, J=8.8 Hz, 1H), 7.59-7.52 (m, 2H), 7.36-7.30 (m, 2H), 7.00 (d, J=8.4 Hz, 1H), 4.71 (s, 2H), 0.91 (s, 9H), 0.09 (s, 6H).

### Step 3: 6-((4-(((Tert-butyldimethylsilyl)oxy)methyl)phenyl)amino)-1′-methyl-5-nitro-[2,3′-bipyridin]-6′(1′H)-one

To a mixture of N-(4-(((tert-butyldimethylsilyl)oxy)methyl)phenyl)-6-chloro-3-nitropyridin-2-amine (900 mg, 2.28 mmol) and (1-methyl-6-oxo-1,6-dihydropyridin-3-yl)boronic acid (591 mg, 2.51 mmol) in 1,4-dioxane (10 mL) and H₂O (2 mL) were added Cs₂CO₃ (2.23 g, 6.85 mmol) and Pd(dppf)Cl₂ (167 mg, 228 μmol). The mixture was stirred at 100° C. for 12 hr under N₂ atmosphere. The mixture was diluted with EtOAc (100 mL) and H₂O (100 mL) and filtered. The filtration cake was concentrated to give 6-((4-(((tert-butyldimethylsilyl)oxy)methyl)phenyl)amino)-1′-methyl-5-nitro-[2,3′-bipyridin]-6′(1′H)-one (600 mg, yield: 56%) as a brown solid. MS: m/z=467.1 [M+H]⁺. ¹H NMR (400 MHz, Dimethylsulfoxide-d₆) δ 10.13 (s, 1H), 8.66 (s, 1H), 8.54 (d, J=8.8 Hz, 1H), 8.06 (d, J=8.4 Hz, 1H), 7.64 (d, J=8.0 Hz, 2H), 7.45-7.32 (m, 3H), 6.47 (d, J=9.6 Hz, 1H), 4.72 (s, 2H), 3.52 (s, 3H), 0.91 (s, 9H), 0.10 (s, 6H).

### Step 4: 5-(2-(2-Aminopyridin-3-yl)-3-(4-(hydroxymethyl)phenyl)-3H-imidazo[4,5-b]pyridin-5-yl)-1-methylpyridin-2(1H)-one

To a mixture of 6-((4-(((tert-butyldimethylsilyl)oxy)methyl)phenyl)amino)-1′-methyl-5-nitro-[2,3′-bipyridin]-6′(1′H)-one (300 mg, 643 μmol) and 2-aminopyridine-3-carbaldehyde (86.4 mg, 707 μmol) in DMSO (3 mL) was added Na₂S20. (280 mg, 1.61 mmol). The mixture was stirred at 100° C. for 12 hr. The mixture was diluted with H₂O (50 mL) and extracted with CH₂Cl₂/MeOH (10/1) (30 mL×3). The combined organic layers were washed with brine (20 mL×3), dried over Na₂SO₄, filtered and concentrated. The mixture was purified by silica gel flash chromatography (Eluent of 0˜60% EtOAc/EtOH (3/1) in petroleum ether) to give 5-(2-(2-aminopyridin-3-yl)-3-(4-(hydroxymethyl)phenyl)-3H-imidazo[4,5-b]pyridin-5-yl)-1-methylpyridin-2(1H)-one (100 mg, yield: 37%) as a yellow solid. MS: m/z=425.0 [M+H]⁺. ¹H NMR (400 MHz, Dimethylsulfoxide-d₆) δ 8.49 (d, J=2.4 Hz, 1H), 8.24 (d, J=8.8 Hz, 1H), 8.08 (dd, J=9.6, 2.4 Hz, 1H), 8.00 (dd, J=4.8, 2.0 Hz, 1H), 7.84 (d, J=8.4 Hz, 1H), 7.50-7.42 (m, 4H), 7.19 (dd, J=8.0, 2.0 Hz, 1H), 6.98 (br s, 2H), 6.50 (d, J=9.6 Hz, 1H), 6.44-6.37 (m, 1H), 5.37 (t, J=6.0 Hz, 1H), 4.61 (d, J=6.0 Hz, 2H), 3.53 (s, 3H).

### Step 5: 5-(2-(2-Aminopyridin-3-yl)-3-(4-(chloromethyl)phenyl)-3H-imidazo[4,5-b]pyridin-5-yl)-1-methylpyridin-2(1H)-one

To a mixture of 5-(2-(2-aminopyridin-3-yl)-3-(4-(hydroxymethyl)phenyl)-3H-imidazo[4,5-b]pyridin-5-yl)-1-methylpyridin-2(1H)-one (100 mg, 236 μmol) in CH₂Cl₂ (2 mL) was added SOCl₂ (84.1 mg, 51.3 μL). The mixture was stirred at 40° C. for 1 hr. The mixture was concentrated to give 5-(2-(2-aminopyridin-3-yl)-3-(4-(chloromethyl)phenyl)-3H-imidazo[4,5-b]pyridin-5-yl)-1-methylpyridin-2(1H)-one (Intermediate 105, 80 mg, yield: 77%) as a yellow solid. MS: m/z=443.0 [M+H]⁺. ¹H NMR (400 MHz, Dimethylsulfoxide-d₆) δ 8.59-8.51 (m, 2H), 8.33 (d, J=8.4 Hz, 1H), 8.17-8.05 (m, 2H), 7.96-7.80 (m, 2H), 7.74-7.48 (m, 3H), 6.94-6.84 (m, 1H), 6.56-6.47 (m, 1H), 4.86 (s, 2H), 3.53 (s, 3H).

### Intermediate 106: 4-(2-(2-aminopyridin-3-yl)-3-(4-(chloromethyl)phenyl)-3H-imidazo[4,5-b]pyridin-5-yl)-1-methylpyridin-2(1H)-one

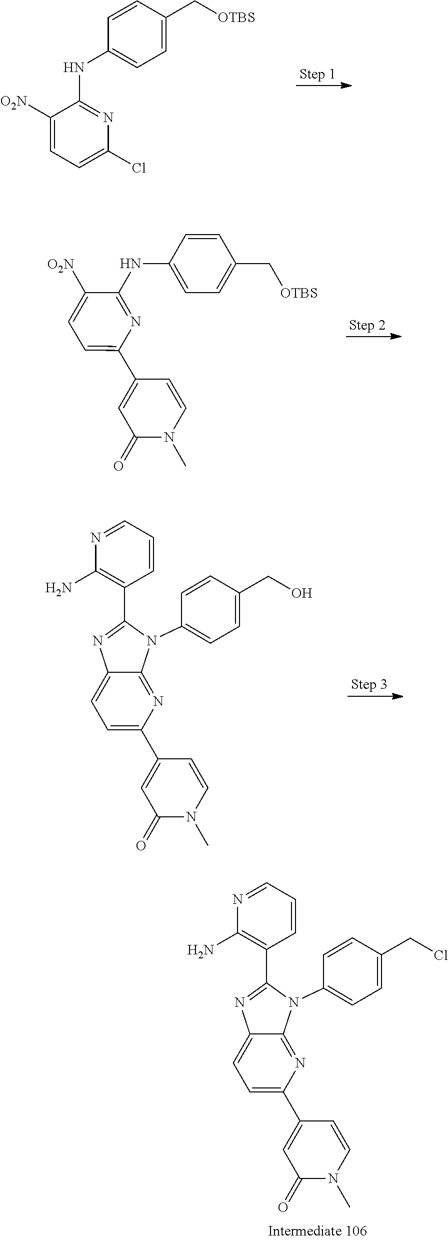 [SMILES: CC(C)(C)[Si](C)(C)OCc1ccc(Nc2nc(Cl)ccc2[N+](=O)[O-])cc1 ; Cn1ccc(-c2ccc([N+](=O)[O-])c(Nc3ccc(CO[Si](C)(C)C(C)(C)C)cc3)n2)cc1=O ; Cn1ccc(-c2ccc3nc(-c4cccnc4N)n(-c4ccc(CO)cc4)c3n2)cc1=O ; Cn1ccc(-c2ccc3nc(-c4cccnc4N)n(-c4ccc(CCl)cc4)c3n2)cc1=O]

### Step 1: 6-((4-(((Tert-butyldimethylsilyl)oxy)methyl)phenyl)amino)-1′-methyl-5-nitro-[2,4′-bipyridin]-2′(1′H)-one

To a mixture of N-(4-(((tert-butyldimethylsilyl)oxy)methyl)phenyl)-6-chloro-3-nitropyridin-2-amine (refer to Intermediate 105 for detail procedures, 6 g, 15.2 mmol) and (1-methyl-2-oxo-4-pyridyl)boronic acid (2.56 g, 16.8 mmol) in H₂O (12 mL) and dioxane (60 mL) were added Pd(dppf)Cl₂ (1.11 g, 1.52 mmol) and Cs₂CO₃ (14.9 g, 45.7 mmol). The mixture was stirred at 100° C. for 2 hr under N₂. H₂O (200 mL) was added and extracted with EtOAc (300 mL×2). The combined organic layers were washed with brine (200 mL), dried over Na₂SO₄, filtered and concentrated. The mixture was purified by silica gel flash chromatography (Eluent 0-50% EtOAc in petroleum ether) to give 6-((4-(((tert-butyldimethylsilyl)oxy)methyl)phenyl)amino)-1′-methyl-5-nitro-[2,4′-bipyridin]-2′(1′H)-one (3.2 g, yield: 45%) as a yellow solid. ¹H NMR (400 MHz, Dimethylsulfoxide-d₆) δ 10.02 (s, 1H), 8.61 (d, J=8.4 Hz, 1H), 7.81 (d, 1=7.2 Hz, 1H), 7.64 (d, J=8.4 Hz, 2H), 7.58 (d, J=8.8 Hz, 1H), 7.34 (d, J=8.4 Hz, 2H), 7.06 (s, 1H), 6.77 (dd, J=6.8, 1.6 Hz, 1H), 4.73 (s, 2H), 3.45 (s, 3H), 0.92 (s, 9H), 0.10 (s, 6H).

### Step 2: 4-(2-(2-Aminopyridin-3-yl)-3-(4-(hydroxymethyl)phenyl)-3H-imidazo[4,5-h]pyridin-5-yl)-1-methylpyridin-2(1H)-one

To a mixture of 6-((4-(((tert-butyldimethylsilyl)oxy)methyl)phenyl)amino)-1′-methyl-5-nitro-[2,4′-bipyridin]-2′(1′H)-one (3.1 g, 6.64 mmol) and 2-aminopyridine-3-carbaldehyde (892 mg, 7.31 mmol) in DMSO (3 mL) was added Na₂S₂O₄ (2.89 g, 16.6 mmol). The mixture was stirred at 100° C. for 12 hr. Sat.NaHCO₃ (500 mL) was added and extracted with CH₂C12/MeOH (10/1) (300 mL×3). The combined organic layers were washed with brine (200 mL×3), dried over Na₂SO₄, filtered and concentrated. The mixture was purified by silica gel flash chromatography (Eluent of 0˜100% EtOAc/EtOH (3/1) in petroleum ether) to give 4-(2-(2-aminopyridin-3-yl)-3-(4-(hydroxymethyl)phenyl)-3H-imidazo[4,5-h]pyridin-5-yl)-1-methylpyridin-2(1H)-one (1.4 g, yield: 50%) as a yellow solid. MS: m/z=425.1 [M+H]⁺. ¹H NMR (400 MHz, Dimethylsulfoxide-d₆) δ 8.28 (d, J=8.0 Hz, 1H), 8.08-7.98 (m, 2H), 7.75 (d, J=7.2 Hz, 1H), 7.54-7.41 (m, 4H), 7.27-7.20 (m, 1H), 7.04 (d, J=1.6 Hz, 1H), 6.97 (d, J=4.4 Hz, 1H), 6.86 (dd, J=7.2, 2.0 Hz, 1H), 6.42 (dd, J=7.6, 4.8 Hz, 1H), 4.64-4.56 (m, 2H), 3.44 (s, 3H).

### Step 3: 4-(2-(2-Aminopyridin-3-yl)-3-(4-(chloromethyl)phenyl)-3H-imidazo[4,5-b]pyridin-5-yl)-1-methylpyridin-2(1H)-one

To a mixture of 4-(2-(2-aminopyridin-3-yl)-3-(4-(hydroxymethyl)phenyl)-3H-imidazo[4,5-b]pyridin-5-yl)-1-methylpyridin-2(1H)-one (400 mg, 942 μmol) in CH₂Cl₂ (5 mL) was added SOCl₂ (336 mg, 205 μL), the mixture was stirred at 40° C. for 1 hr. The mixture was concentrated to give 4-(2-(2-aminopyridin-3-yl)-3-(4-(chloromethyl)phenyl)-3H-imidazo[4,5-b]pyridin-5-yl)-1-methylpyridin-2(1H)-one (Intermediate 106, 400 mg, yield: 96%) as a yellow solid. MS: m/z=443.1 [M+H]⁺. ¹H NMR (400 MHz, Dimethylsulfoxide-d₆) δ 8.50-8.36 (m, 2H), 8.17-8.10 (m, 2H), 7.95 (dd, J=7.6, 1.6 Hz, 1H), 7.81 (d, J=7.2 Hz, 1H), 7.71-7.56 (m, 4H), 7.13-7.07 (m, 1H), 6.97-6.86 (m, 2H), 4.88 (s, 2H), 3.46 (s, 3H).

### Intermediate 107: 3-(3-(4-(Chloromethyl)phenyl)-5-(4-fluorophenyl)-3H-imidazo[4,5-b]pyridin-2-yl)pyridin-2-amine

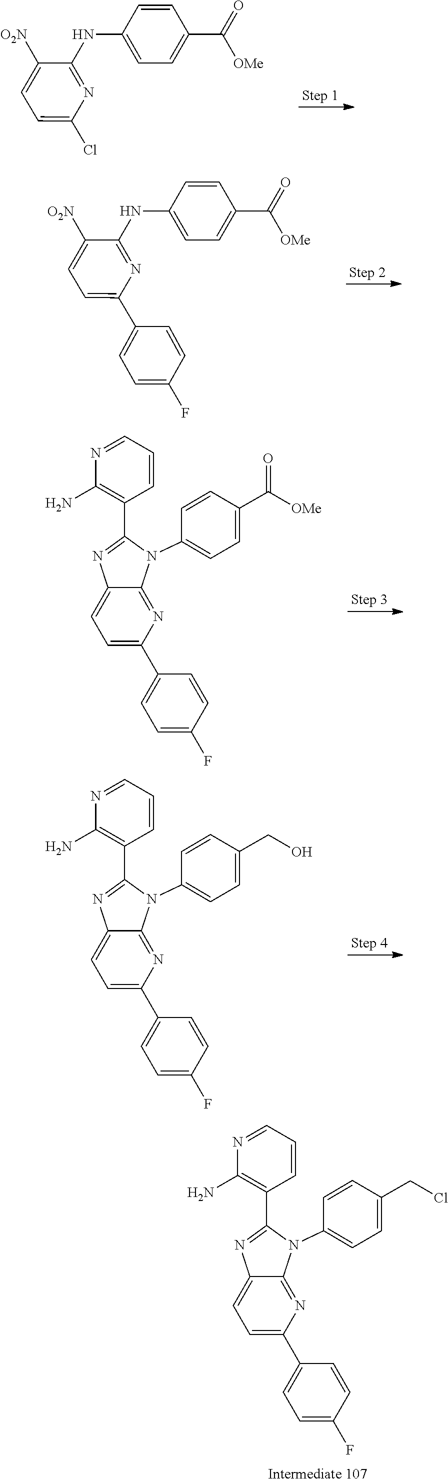 [SMILES: COC(=O)c1ccc(Nc2nc(Cl)ccc2[N+](=O)[O-])cc1 ; COC(=O)c1ccc(Nc2nc(-c3ccc(F)cc3)ccc2[N+](=O)[O-])cc1 ; COC(=O)c1ccc(-n2c(-c3cccnc3N)nc3ccc(-c4ccc(F)cc4)nc32)cc1 ; Nc1ncccc1-c1nc2ccc(-c3ccc(F)cc3)nc2n1-c1ccc(CO)cc1 ; Nc1ncccc1-c1nc2ccc(-c3ccc(F)cc3)nc2n1-c1ccc(CCl)cc1]

### Step 1: Methyl 4-((6-(4-fluorophenyl)-3-nitropyridin-2-yl)amino)benzoate

A mixture of methyl 4-((6-chloro-3-nitropyridin-2-yl)amino)benzoate (refer to Intermediate 13 for detail procedures, 5.0 g, 16.3 mmol), (4-fluorophenyl)boronic acid (3.0 g, 21.1 mmol), Pd(dppf)Cl₂ (1.2 g, 1.63 mmol), and Cs₂CO₃ (15.9 g, 48.8 mmol) in 1,4-dioxane (50 mL) and H₂O (5 mL) was degassed and purged with N₂ three times, and then the mixture was stirred at 100° C. for 16 hr under N₂ atmosphere. The reaction mixture was diluted with water (200 mL) and extracted with EtOAc (300 mL×3). The combined organic layers were dried over Na₂SO₄, filtered and concentrated under reduced pressure. The residue was purified by silica gel flash chromatography (Eluent of 0˜10% MeOH in CH₂Cl₂) to give methyl 4-((6-(4-fluorophenyl)-3-nitropyridin-2-yl)amino)benzoate (2.5 g, yield: 34%) as a yellow solid. MS: m/z=340.3 [M+H]⁺. ¹H NMR (400 MHz, Dimethylsulfoxide-d₆) δ 10.45 (s, 1H), 8.62 (d, J=8.6 Hz, 1H), 8.15-8.04 (m, 4H), 7.85 (d, J=8.4 Hz, 2H), 7.34 (d, J=8.6 Hz, 1H), 7.24-7.18 (m, 2H), 3.95 (s, 3H).

### Step 2: Methyl 4-(2-(2-aminopyridin-3-yl)-5-(4-fluorophenyl)-3H-imidazo[4,5-b]pyridin-3-yl)benzoate

To a solution of methyl 4-((6-(4-fluorophenyl)-3-nitropyridin-2-yl)amino)benzoate (2.5 g, 6.81 mmol) and 2-aminopyridine-3-carbaldehyde (914 mg, 7.49 mmol) in DMSO (100 mL) was added Na₂S₂O₄ (4.74 g, 7.49 mmol). The mixture was stirred at 100° C. for 12 hr. The reaction mixture was diluted with water (100 mL) and extracted with CH₂Cl₂ (300 mL×2). The combined organic layers were washed with brine (100 mL), dried over Na₂SO₄, filtered and concentrated under reduced pressure. The residue was purified by silica gel flash chromatography (Eluent of 0˜100% EtOAc in petroleum ether) to give methyl 4-(2-(2-aminopyridin-3-yl)-5-(4-fluorophenyl)-3H-imidazo[4,5-b]pyridin-3-yl)benzoate (1.3 g, yield: 40%) as yellow solid. MS: m/z=440.0 [M+H]⁺. ¹H NMR (400 MHz, Chloroform-d) δ 8.26-8.21 (m, 2H), 8.15 (d, J=8.4 Hz, 1H), 8.08 (dd, J=5.2, 1.6 Hz, 1H), 8.03-7.98 (m, 2H), 7.79 (d, J=8.4 Hz, 1H), 7.56 (d, J=8.4 Hz, 2H), 7.17-7.08 (m, 3H), 6.82 (br s, 2H), 6.42 (dd, J=7.6, 4.8 Hz, 1H), 3.99 (s, 3H).

### Step 3: (4-(2-(2-Aminopyridin-3-yl)-5-(4-fluorophenyl)-3H-imidazo[4,5-b]pyridin-3-yl)phenyl)methanol

To a solution of methyl 4-(2-(2-aminopyridin-3-yl)-5-(4-fluorophenyl)-3H-imidazo[4,5-b]pyridin-3-yl)benzoate (1.3 g, 2.92 mmol) in THF (100 mL) was added LiAlH₄ (2.5 M, 4.44 mmol) at 0° C. The mixture was stirred at 0° C. for 2 hr. The reaction mixture was quenched with Na₂SO₄·10H₂O (203 mg) at 0° C. and filtered. The filtrate was concentrated under reduced pressure. The crude (4-(2-(2-aminopyridin-3-yl)-5-(4-fluorophenyl)-3H-imidazo[4,5-b]pyridin-3-yl)phenyl)methanol (1.2 g) was used in the next step without further purification. MS: m/z=412.0 [M+H]⁺.

### Step 4: 3-(3-(4-(Chloromethyl)phenyl)-5-(4-fluorophenyl)-3H-imidazo[4,5-b]pyridin-2-yl)pyridin-2-amine

To a solution of (4-(2-(2-aminopyridin-3-yl)-5-(4-fluorophenyl)-3H-imidazo[4,5-b]pyridin-3-yl)phenyl)methanol (1.2 g, 2.91 mmol) in CH₂Cl₂ (20 mL) was added SOCl₂ (2.1 g, 17.5 mmol). The mixture was stirred at 40° C. for 2 hr. The mixture was concentrated under reduced pressure. 3-(3-(4-(Chloromethyl)phenyl)-5-(4-fluorophenyl)-3H-imidazo[4,5-b]pyridin-2-yl)pyridin-2-amine (Intermediate 107, 1.25 g HCl salt, yield: 89%) was obtained as a yellow solid. MS: m/z=429.9 [M+H]⁺.

### Intermediate 108: 3-(3-(4-(Chloromethyl)phenyl)-5-(3-chlorophenyl)-3H-imidazo[4,5-b]pyridin-2-yl)pyridin-2-amine

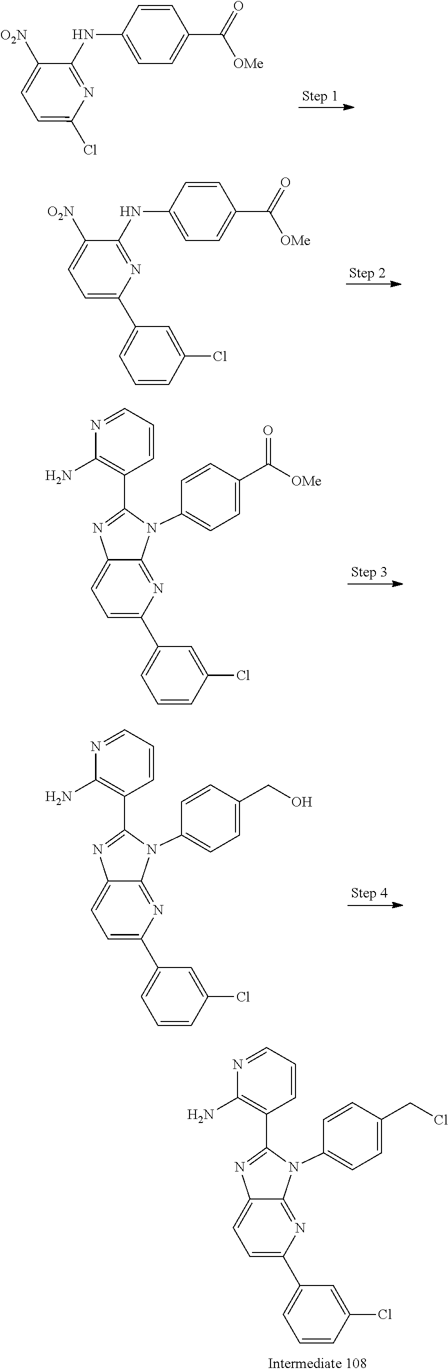

### Step 1: Methyl 4-((6-(3-chlorophenyl)-3-nitropyridin-2-yl)amino)benzoate

A mixture of methyl 4-((6-chloro-3-nitropyridin-2-yl)amino)benzoate (refer to Intermediate 13 for detail procedures, 5.0 g, 16.2 mmol), (3-chlorophenyl)boronic acid (3.3 g, 21.1 mmol), Pd(dppf)Cl₂ (1.2 g, 1.63 mmol), Cs₂CO₃ (16 g, 48.8 mmol) in 1,4-dioxane (50 mL) and H₂O (5 mL) was degassed and purged with N₂ three times, and the mixture was stirred at 100° C. for 12 hr under N₂ atmosphere. The reaction mixture was diluted with water (20 mL) and extracted with CH₂Cl₂ (30 mL×2). The combined organic layers were dried over Na₂SO₄, filtered and concentrated under reduced pressure. The residue was purified by silica gel flash chromatography (Eluent of 0˜10% MeOH in CH₂Cl₂) to give methyl 4-((6-(3-chlorophenyl)-3-nitropyridin-2-yl)amino)benzoate (6 g, yield: 84%) as a yellow solid. MS: m/z=383.9 [M+H]⁺. ¹H NMR (400 MHz, Chloroform-d) δ 10.44 (br s, 1H), 8.64 (d, J=8.4 Hz, 1H), 8.12 (d, J=8.4 Hz, 2H), 8.04 (s, 1H), 7.94 (d, J=7.6 Hz, 1H), 7.86 (d, J=8.4 Hz, 2H), 7.53-7.43 (m, 2H), 7.36 (d, J=8.6 Hz, 1H), 3.95 (s, 3H).

### Step 2: Methyl 4-(2-(2-aminopyridin-3-yl)-5-(3-chlorophenyl)-3H-imidazo[4,5-b]pyridin-3-yl)benzoate

To a solution of methyl 4-((6-(3-chlorophenyl)-3-nitropyridin-2-yl)amino)benzoate (5.5 g, 14.3 mmol) and 2-aminonicotinaldehyde (1.9 g, 15.7 mmol) in DMSO (200 mL) was added Na₂S₂O₄ (10 g, 57.3 mmol). The mixture was stirred at 100° C. for 12 hr. The reaction mixture was diluted with water (100 mL) and extracted with CH₂Cl₂ (300 mL×2). The combined organic layers were washed with brine (100 mL), dried over Na₂SO₄, filtered and concentrated under reduced pressure. The residue was purified by silica gel flash chromatography (Eluent of 0˜100% EtOAc in petroleum ether) to give methyl 4-(2-(2-aminopyridin-3-yl)-5-(3-chlorophenyl)-3H-imidazo[4,5-b]pyridin-3-yl)benzoate (2.4 g, yield: 52%) as a yellow solid. MS: m/z=456.0 [M+H]⁺. ¹H NMR (400 MHz, Chloroform-d) δ 8.22 (d, J=8.4 Hz, 2H), 8.14 (d, J=8.4 Hz, 1H), 8.11-8.08 (m, 1H), 7.98 (s, 1H), 7.89-7.86 (m, 1H), 7.78 (d, J=8.4 Hz, 1H), 7.54 (d, J=8.6 Hz, 2H), 7.38-7.32 (m, 2H), 7.05 (dd, J=7.6, 1.6 Hz, 1H), 6.60 (br s, 2H), 6.39 (dd, J=7.6, 4.8 Hz, 1H), 3.98 (s, 3H).

### Step 3: (4-(2-(2-Aminopyridin-3-yl)-5-(3-chlorophenyl)-3H-imidazo[4,5-b]pyridin-3-yl)phenyl)methanol

To a solution of methyl 4-(2-(2-aminopyridin-3-yl)-5-(3-chlorophenyl)-3H-imidazo[4,5-b]pyridin-3-yl)benzoate (2.4 g, 5.26 mmol) in THF (100 mL) was added LiAlH₄ (2.5 M, 1.05 mL) at 0° C. The mixture was stirred at 0° C. for 2 hr. The reaction mixture was quenched with Na₂SO₄·10H₂O (300 mg) at 0° C. and filtered. The filtrate was concentrated under reduced pressure to give (4-(2-(2-aminopyridin-3-yl)-5-(3-chlorophenyl)-3H-imidazo[4,5-b]pyridin-3-yl)phenyl)methanol (2.2 g, yield. 85%) as a yellow solid. MS: m/z=430.0 [M+H]⁺.

### Step 4: 3-(3-(4-(Chloromethyl)phenyl)-5-(3-chlorophenyl)-3H-imidazo[4,5-b]pyridin-2-yl)pyridin-2-amine

To a solution of (4-(2-(2-aminopyridin-3-yl)-5-(3-chlorophenyl)-3H-imidazo[4,5-b]pyridin-3-yl)phenyl)methanol (2.2 g, 5.14 mmol) in CH₂Cl₂ (25 mL) was added SOCl₂ (3.67 g, 30.9 mmol). The mixture was stirred at 40° C. for 2 hr. The mixture was concentrated under reduced pressure to give 3-(3-(4-(chloromethyl)phenyl)-5-(3-chlorophenyl)-3H-imidazo[4,5-b]pyridin-2-yl)pyridin-2-amine (Intermediate 108, 2.2 g HCl salt, yield: 84%) as a yellow solid. MS: m/z=446.0 [M+H]⁺.

### Intermediate 109: 3-(3-(4-(((1S,4S)-2,5-Diazabicyclo[2.2.1]heptan-2-yl)methyl)phenyl)-5-phenyl-3H-imidazo[4,5-b]pyridin-2-yl)pyridin-2-amine

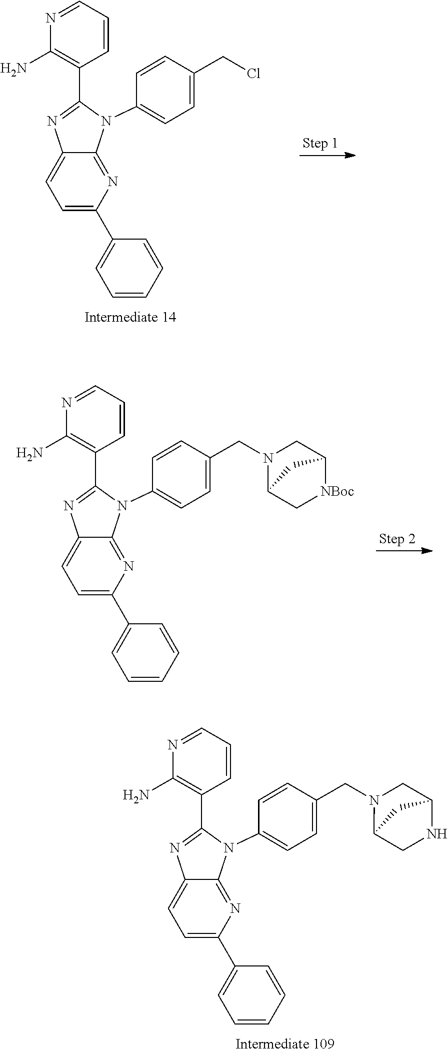 [SMILES: Nc1ncccc1-c1nc2ccc(-c3ccccc3)nc2n1-c1ccc(CCl)cc1 ; CC(C)(C)OC(=O)N1C[C@@H]2C[C@H]1CN2Cc1ccc(-n2c(-c3cccnc3N)nc3ccc(-c4ccccc4)nc32)cc1 ; Nc1ncccc1-c1nc2ccc(-c3ccccc3)nc2n1-c1ccc(CN2C[C@@H]3C[C@H]2CN3)cc1]

### Step 1: Tert-butyl (1S,4S)-5-(4-(2-(2-aminopyridin-3-yl)-5-phenyl-3H-imidazo[4,5-b]pyridin-3-yl)benzyl)-2,5-diazabicyclo[2.2.1]heptane-2-carboxylate

To a solution of Intermediate 14 (1.0 g, 2.43 mmol) in DMF (10 mL) were added DIEA (1.27 mL, 7.28 mmol) and tert-butyl (1S,4S)-2,5-diazabicyclo[2.2.1]heptane-2-carboxylate (530 mg, 2.67 mmol). The mixture was degassed and purged with N₂ three times and stirred at 80° C. for 16 hr under N₂. The reaction mixture was diluted with CH₂Cl₂ (80 mL) at 25° C., washed with brine (50 mL×3), dried over Na₂SO₄, filtered and concentrated under reduced pressure. The residue was purified by silica gel flash chromatography (Eluent of 1˜4% MeOH in CH₂Cl₂) to give tert-butyl (1S,4S)-5-(4-(2-(2-aminopyridin-3-yl)-5-phenyl-3H-imidazo[4,5-b]pyridin-3-yl)benzyl)-2,5-diazabicyclo[2.2.1]heptane-2-carboxylate (1.14 g, yield: 75%) as a yellow solid. MS: m/z=574.4 [M+H]⁺. ¹H NMR (400 MHz, Chloroform-d) 58.12 (d, J=8.4 Hz, 1H), 8.06 (dd, J=4.8, 1.6 Hz, 1H), 8.04-7.98 (m, 2H), 7.80 (d, J=8.4 Hz, 1H), 7.50 (d, J=8.4 Hz, 2H), 7.47-7.33 (m, 5H), 7.10 (d, J=7.6 Hz, 1H), 6.60 (br s, 2H), 6.36 (dd, J=8.0, 4.8 Hz, 1H), 4.47-4.24 (m, 1H), 3.88-3.80 (m, 2H), 3.71-3.49 (m, 2H), 3.28-3.16 (m, 1H), 3.02-2.89 (m, 1H), 2.82-2.57 (m, 1H), 1.96-1.87 (m, 1H), 1.77-1.68 (m, 1H), 1.48 (s, 9H).

### Step 2: 3-(3-(4-(((1S,4S)-2,5-Diazabicyclo[2.2.1]heptan-2-yl)methyl)phenyl)-5-phenyl-3H-imidazo[4,5-b]pyridin-2-yl)pyridin-2-amine

To a solution of tert-butyl (1S,4S)-5-(4-(2-(2-aminopyridin-3-yl)-5-phenyl-3H-imidazo[4,5-b]pyridin-3-yl)benzyl)-2,5-diazabicyclo[2.2.1]heptane-2-carboxylate (50 mg, 87.2 μmol) in dioxane (0.5 mL) was added HCl in 1,4-dioxane (4 M, 1 mL). The mixture was degassed and purged with N₂ three times and stirred at 25° C. for 2 hr under N₂. The reaction mixture was concentrated under reduced pressure. The residue was triturated with 1 mL 1,4-dioxane at 25° C. for 10 min. 3-(3-(4-(((1S,4S)-2,5-Diazabicyclo[2.2.1]heptan-2-yl)methyl)phenyl)-5-phenyl-3H-imidazo[4,5-b]pyridin-2-yl)pyridin-2-amine (Intermediate 109, 41 mg, HCl salt, yield: 95%) was obtained as a light-yellow solid. MS: m/z=474.2 [M+H]⁺. ¹H NMR (400 MHz, Dimethylsulfoxide-d₆) δ 12.43-11.57 (m, 1H), 10.39-9.51 (m, 2H), 8.55-8.39 (m, 1H), 8.38 (d, J=8.4 Hz, 1H), 8.14 (d, J=5.2 Hz, 1H), 8.11-8.01 (m, 3H), 7.94 (d, J=8.0 Hz, 2H), 7.84 (d, J=7.2 Hz, 1H), 7.69 (d, J=8.4 Hz, 2H), 7.54-7.40 (m, 3H), 6.91-6.87 (m, 1H), 4.68-4.40 (m, 4H), 3.99-3.94 (m, 1H), 3.80-3.75 (m, 2H), 3.42-3.40 (m, 1H), 2.60-2.52 (m, 1H), 2.20-2.06 (m, 1H).

### Intermediate 110: 3-(3-(4-((4-Aminopiperidin-1-yl)methyl)phenyl)-2-(2-aminopyridin-3-yl)-3H-imidazo[4,5-b]pyridin-5-yl)pyridin-2(1H)-one

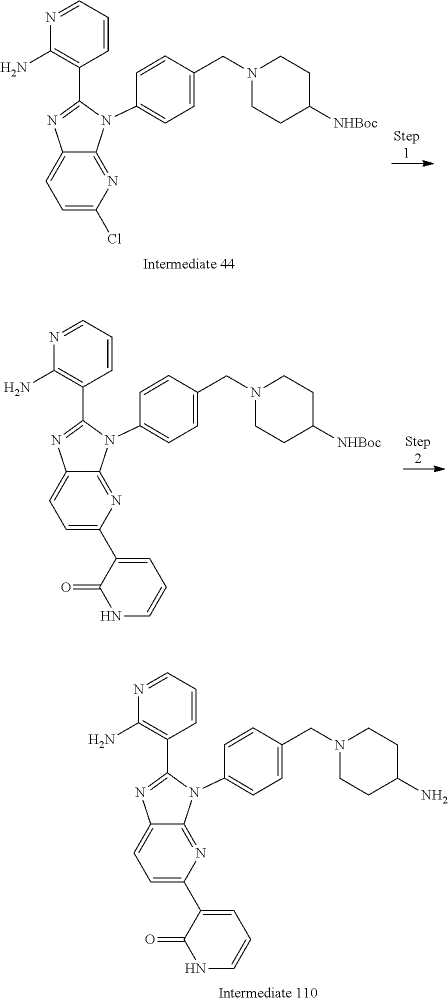 [SMILES: CC(C)(C)OC(=O)NC1CCN(Cc2ccc(-n3c(-c4cccnc4N)nc4ccc(Cl)nc43)cc2)CC1 ; CC(C)(C)OC(=O)NC1CCN(Cc2ccc(-n3c(-c4cccnc4N)nc4ccc(-c5ccc[nH]c5=O)nc43)cc2)CC1 ; Nc1ncccc1-c1nc2ccc(-c3ccc[nH]c3=O)nc2n1-c1ccc(CN2CCC(N)CC2)cc1]

### Step 1: tert-Butyl (1-(4-(2-(2-aminopyridin-3-yl)-5-(2-oxo-1,2-dihydropyridin-3-yl)-3H-imidazo[4,5-b]pyridin-3-yl)benzyl)piperidin-4-yl)carbamate

A mixture of Intermediate 44 (200 mg, 375 μmol), (2-oxo-1,2-dihydropyridin-3-yl)boronic acid (78 mg, 562 μmol), Cs₂CO₃ (244 mg, 749 μmol), and Pd(dppf)Cl₂ (27 mg, 37 μmol) in 1,4-dioxane (5 mL) and H₂O (1 mL) was degassed and purged with N₂ three times, and then the mixture was stirred at 100° C. for 16 hr under N₂ atmosphere. The mixture was diluted with H₂O (30 mL) and extracted with EtOAc (30 mL/2). The combined organic layers were washed with brine (30 mL), dried over Na₂SO₄, filtered and concentrated under reduced pressure. The crude was purified by silica gel flash chromatography (Eluent of 0˜10/MeOH in CH₂Cl₂) to give tert-butyl (1-(4-(2-(2-aminopyridin-3-yl)-5-(2-oxo-1,2-dihydropyridin-3-yl)-3H-imidazo[4,5-b]pyridin-3-yl)benzyl)piperidin-4-yl)carbamate (90 mg, yield 41%) as a light-yellow solid. MS: m/z=593.3 [M+H]⁺. ¹H NMR (400 MHz, Chloroform-d) δ 12.66 (br s, 1H), 8.65 (d, J=7.6 Hz, 1H), 8.40 (dd, J=7.2, 1.6 Hz, 1H), 8.13 (d, J=8.4 Hz, 1H), 8.06 (dd, J=4.4, 1.2 Hz, 1H), 7.52-7.45 (m, 3H), 7.37 (d, J=8.0 Hz, 2H), 7.08 (dd, J=7.6, 1.2 Hz, 1H), 6.68 (br s, 2H), 6.50-6.45 (m, 1H), 6.34 (dd, J=7.6, 4.8 Hz, 1H), 4.52-4.44 (m, 1H), 3.60 (s, 2H), 3.52-3.47 (m, 1H), 2.91-2.84 (m, 2H), 2.21-2.14 (m, 2H), 1.99-1.93 (m, 2H), 1.55-1.48 (m, 2H), 1.45 (s, 9H).

### Step 2: 3-(3-(4-((4-Aminopiperidin-1-yl)methyl)phenyl)-2-(2-aminopyridin-3-yl)-3H-imidazo[4,5-b]pyridin-5-yl)pyridin-2(1H)-one

A solution of tert-butyl (1-(4-(2-(2-aminopyridin-3-yl)-5-(2-oxo-1,2-dihydropyridin-3-yl)-3H-imidazo[4,5-b]pyridin-3-yl)benzyl)piperidin-4-yl)carbamate (90 mg, 152 μmol) in HCl in 1,4-dioxane (4 M, 2 mL) was stirred at 25° C. for 1 hr. The mixture was concentrated under reduced pressure to give 3-(3-(4-((4-aminopiperidin-1-yl)methyl)phenyl)-2-(2-aminopyridin-3-yl)-3H-imidazo[4,5-b]pyridin-5-yl)pyridin-2(1H)-one (Intermediate 110, 63.5 mg, yield: 78%) as a yellow solid. MS: m/z=493.2 [M+H]⁺. ¹H NMR (400 MHz, Methanol-d₄) δ 8.67-8.62 (m, 1H), 8.55 (d, J=8.4 Hz, 1H), 8.43 (d, J=8.0 Hz, 1H), 8.05-8.03 (m, 1H), 7.96-7.88 (m, 3H), 7.80 (d, J=5.2 Hz, 1H), 7.75-7.74 (m, 2H), 6.94-6.84 (m, 2H), 4.52 (s, 2H), 3.78-3.62 (m, 3H), 3.61-3.49 (m, 2H), 2.35-2.28 (m, 2H), 2.20-2.10 (m, 2H).

### Intermediate 111: 4-(4-(2-(2-Aminopyridin-3-yl)-5-phenyl-3H-imidazo[4,5-b]pyridin-3-yl)benzyl)piperazin-1-amine hydrochloride

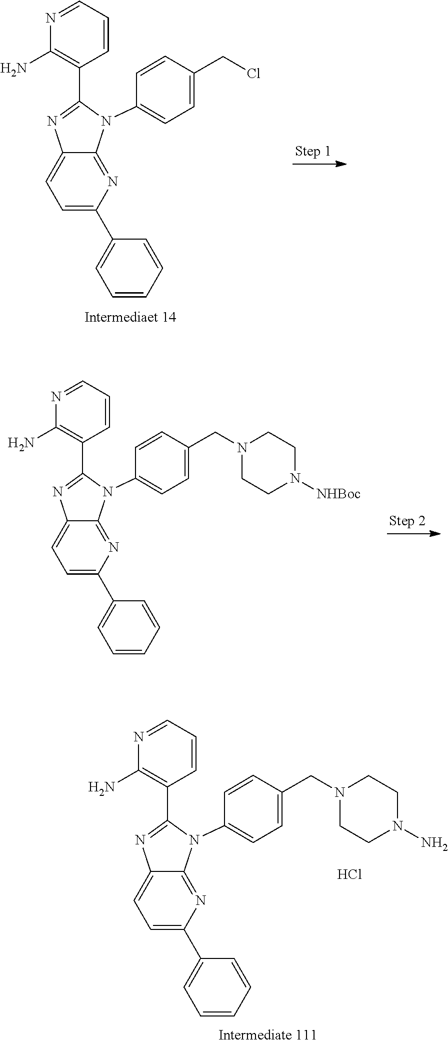 [SMILES: Nc1ncccc1-c1nc2ccc(-c3ccccc3)nc2n1-c1ccc(CCl)cc1 ; CC(C)(C)OC(=O)NN1CCN(Cc2ccc(-n3c(-c4cccnc4N)nc4ccc(-c5ccccc5)nc43)cc2)CC1 ; Nc1ncccc1-c1nc2ccc(-c3ccccc3)nc2n1-c1ccc(CN2CCN(N)CC2)cc1 ; Cl]

### Step 1: Tert-butyl (4-(4-(2-(2-aminopyridin-3-yl)-5-phenyl-3H-imidazo[4,5-b]pyridin-3-yl)benzyl)piperazin-1-yl)carbamate

To a solution of Intermediate 14 (1.50 g, 3.64 mmol) in DMF (5 mL) were added K₂CO₃ (1.01 g, 7.28 mmol), NaI (109 mg, 728 μmol), and tert-butyl piperazin-1-ylcarbamate (806 mg, 4.01 mmol) at 25° C. This mixture was stirred at 60° C. for 5 hr. H₂O (35 mL) was added to the mixture. The aqueous phase was extracted with EtOAc (30 mL×3). The combined organic layers were washed with brine (30 mL), dried over anhydrous Na₂SO₄, filtered and concentrated. The crude was purified by silica gel flash chromatography (Eluent of 0˜10% MeOH in CH₂Cl₂) to give tert-butyl(4-(4-(2-(2-aminopyridin-3-yl)-5-phenyl-3H-imidazo[4,5-b]pyridin-3-yl)benzyl)piperazin-1-yl)carbamate (1.65 g, yield: 79¹%) as yellow oil. MS: m/z=577.3 [M+H]⁺.

### Step 2: 4-(4-(2-(2-Aminopyridin-3-yl)-5-phenyl-3H-imidazo[4,5-b]pyridin-3-yl)benzyl)piperazin-1-amine hydrochloride

To a solution of tert-butyl(4-(4-(2-(2-aminopyridin-3-yl)-5-phenyl-3H-imidazo[4,5-b]pyridin-3-yl)benzyl)piperazin-1-yl)carbamate (800 mg, 1.39 mmol) in 1,4-dioxane (10 mL) was added HCl/1,4-dioxane (4 M, 347 μL) at 25° C. This mixture was stirred at 25° C. for 12 hr. The mixture was filtered and concentrated to give 4-(4-(2-(2-aminopyridin-3-yl)-5-phenyl-3H-imidazo[4,5-b]pyridin-3-yl)benzyl)piperazin-1-amine hydrochloride (Intermediate 111, 700 mg, HCl salt, yield: 98%) as a yellow solid, which was used in the next step without purification. MS: m/z=477.3 [M+H]+.

### Intermediate 112: 3-(2-(2-Aminopyridin-3-yl)-3-(4-(chloromethyl)phenyl)-3H-imidazo[4,5-b]pyridin-5-yl)isonicotinonitrile

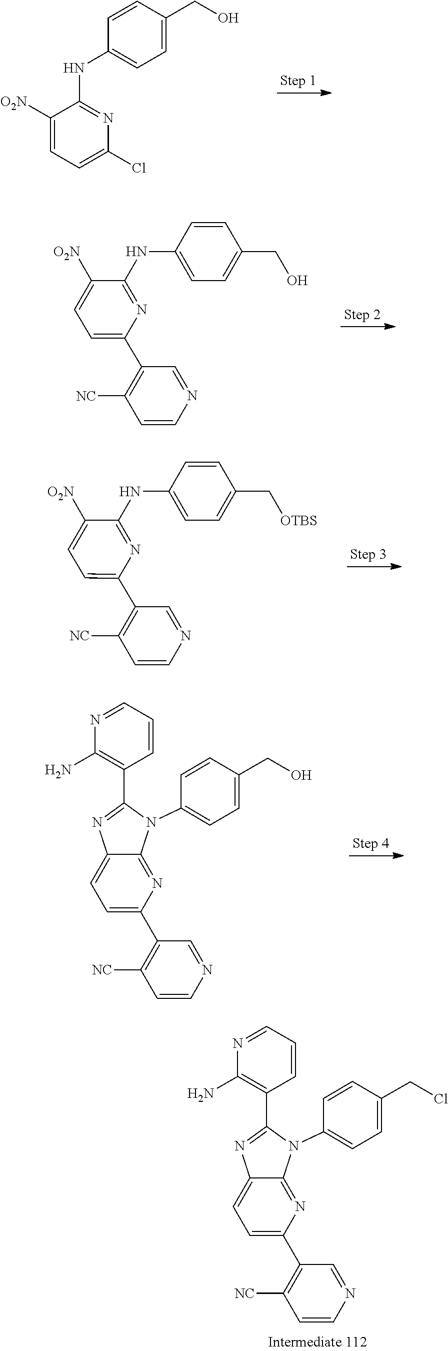

### Step 1: 6-((4-(Hydroxymethyl)phenyl)amino)-5-nitro-[2,3′-bipyridine]-4′-carbonitrile

A mixture of (4-((6-chloro-3-nitropyridin-2-yl)amino)phenyl)methanol (refer to Intermediate 105 for detail procedures, 2.0 g, 7.0 mmol), 3-(4,4,5,5-tetramethyl-1,3,2-dioxaborolan-2-yl)pyridine-4-carbonitrile (1.8 g, 7.9 mmol), cataCXiumAPdG3 (2.6 g, 3.6 mmol), PCy₃ (200 mg, 715 μmol), and K₃PO₄ (3.0 g, 14.0 mmol) in DMF (20 mL) was degassed and purged with N₂ three times, and then mixture was stirred at 120° C. for 2 hr under N₂ atmosphere. The reaction mixture was diluted with water (100 mL) and extracted with EtOAc (150 mL×2). The combined organic layers were washed with brine (100 mL), dried over Na₂SO₄, filtered and concentrated under reduced pressure. After purified by silica gel flash chromatography (Eluent of 0˜26% EtOAc in petroleum ether), 6-((4-(hydroxymethyl)phenyl)amino)-5-nitro-[2,3′-bipyridine]-4′-carbonitrile (1.9 g, yield: 58%) was obtained as an orange solid. MS: m/z=348.0 [M+H]⁺, ¹H NMR (400 MHz, Dimethylsulfoxide-d₆) δ 10.07 (s, 1H), 9.10 (s, 1H), 8.93 (d, J=5.2 Hz, 1H), 8.74 (d, J=8.4 Hz, 1H), 8.04 (d, J=5.2 Hz, 1H), 7.67 (d, J=8.4 Hz, 2H), 7.48 (d, J=8.4 Hz, 1H), 7.29 (d, J=8.4 Hz, 2H), 5.16 (t, J=6.0 Hz, 1H), 4.47 (d, J=6.4 Hz, 2H).

### Step 2: 6-((4-(((Tert-butyldimethylsilyl)oxy)methyl)phenyl)amino)-5-nitro-[2,3′-bipyridine]-4′-carbonitrile

A mixture of 6-((4-(hydroxymethyl)phenyl)amino)-5-nitro-[2,3′-bipyridine]-4′-carbonitrile (1.9 g, 5.5 mmol), TBSCl (1.65 g, 11 mmol) and imidazole (931 mg, 14 mmol) in CH₂Cl₂ (20 mL) was degassed and purged with N₂ three times, and then the mixture was stirred at 25° C. for 2 hr under N₂ atmosphere. The reaction mixture was diluted with water (100 mL) and extracted with EtOAc (150 mL×2). The combined organic layers were washed with brine (100 mL), dried over Na₂SO₄, filtered and concentrated under reduced pressure. After purified by silica gel flash chromatography (Eluent of 0˜5% MeOH in CH₂Cl₂), 6-((4-(((tert-butyldimethylsilyl)oxy)methyl)phenyl)amino)-5-nitro-[2,3′-bipyridine]-4′-carbonitrile (1.2 g, yield: 34%) was obtained as an orange solid. MS: m/z=462.1 [M+H]⁺, ¹H NMR (400 MHz, Dimethylsulfoxide-d₆) δ 10.08 (s, 1H), 9.11 (s, 1H), 8.93 (d, J=5.2 Hz, 1H), 8.74 (d, J=8.4 Hz, 1H), 8.04 (d, J=5.4 Hz, 1H), 7.68 (d, J=8.4 Hz, 2H), 7.48 (d, J=8.4 Hz, 1H), 7.28 (d, J=8.4 Hz, 2H), 4.69 (s, 2H), 0.89 (s, 9H), 0.07 (s, 6H).

### Step 3: 3-(2-(2-Aminopyridin-3-yl)-3-(4-(hydroxymethyl)phenyl)-3H-imidazo[4,5-b]pyridin-5-yl)isonicotinonitrile

A mixture of 6-((4-(((tert-butyldimethylsilyl)oxy)methyl)phenyl)amino)-5-nitro-[2,3′-bipyridine]-4′-carbonitrile (1.2 g, 2.6 mmol), 2-aminopyridine-3-carbaldehyde (349 mg, 2.9 mmol), Na₂S₂O₄ (1.8 g, 10 mmol) in DMSO (50 mL) was degassed and purged with N₂ three times, and then the mixture was stirred at 100° C. for 16 hr under N₂ atmosphere. The reaction mixture was diluted with water (100 mL) and extracted with EtOAc (300 mL×2). The combined organic layers were washed with brine (200 mL), dried over Na₂SO₄, filtered and concentrated under reduced pressure. After purified by silica gel flash chromatography (Eluent of 0˜5% MeOH in CH₂Cl₂), 3-(2-(2-aminopyridin-3-yl)-3-(4-(hydroxymethyl)phenyl)-3H-imidazo[4,5-b]pyridin-5-yl)isonicotinonitrile (600 mg, yield: 22%) was obtained as a red solid. MS: m/z=420.0 [M+H]⁺.

### Step 4: 3-(2-(2-Aminopyridin-3-yl)-3-(4-(chloromethyl)phenyl)-3H-imidazo[4,5-b]pyridin-5-yl)isonicotinonitrile

To a solution of 3-(2-(2-aminopyridin-3-yl)-3-(4-(hydroxymethyl)phenyl)-3H-imidazo[4,5-b]pyridin-5-yl)isonicotinonitrile (600 mg, 715 μmol) in CH₂Cl₂ (20 mL) was added SOCl₂ (511 mg, 4.3 mmol). The mixture was stirred at 40° C. for 2 hr. The reaction mixture was diluted with water (20 mL) and adjusted the pH to about 7-8 by saturated NaHCO₃ solution, then extracted with EtOAc (50 mL×2). The combined organic layers were washed with brine (30 mL), dried over Na₂SO₄, filtered and concentrated under reduced pressure. After purified by silica gel flash chromatography (Eluent of 0˜26% EtOAc in petroleum ether), 3-(2-(2-aminopyridin-3-yl)-3-(4-(chloromethyl)phenyl)-3H-imidazo[4,5-b]pyridin-5-yl)isonicotinonitrile (Intermediate 112, 202 mg, yield: 55%) was obtained as a yellow solid. MS: m/z=437.9 [M+H]⁺.

### Intermediate 113: 3-(3-(4-((4-Aminopiperidin-1-yl)methyl)phenyl)-5-(3-chlorophenyl)-3H-imidazo[4,5-b]pyridin-2-yl)pyridin-2-amine

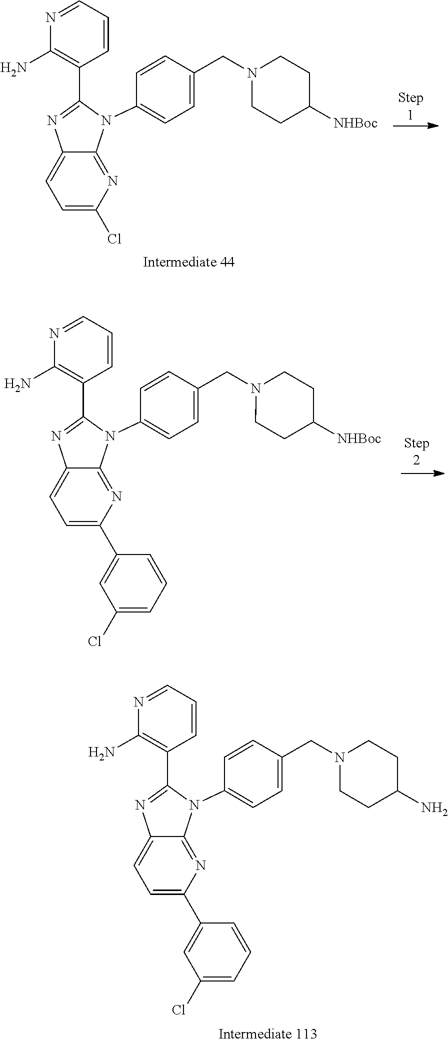 [SMILES: CC(C)(C)OC(=O)NC1CCN(Cc2ccc(-n3c(-c4cccnc4N)nc4ccc(Cl)nc43)cc2)CC1 ; CC(C)(C)OC(=O)NC1CCN(Cc2ccc(-n3c(-c4cccnc4N)nc4ccc(-c5cccc(Cl)c5)nc43)cc2)CC1 ; Nc1ncccc1-c1nc2ccc(-c3cccc(Cl)c3)nc2n1-c1ccc(CN2CCC(N)CC2)cc1]

### Step 1: tert-Butyl (1-(4-(2-(2-aminopyridin-3-yl)-5-(3-chlorophenyl)-3H-imidazo[4,5-b]pyridin-3-yl)benzyl)piperidin-4-yl)carbamate

A mixture of Intermediate 44 (200 mg, 375 μmol), (3-chlorophenyl)boronic acid (88 mg, 562 mol), Pd(dppf)Cl₂ (27 mg, 37 μmol), and Cs₂CO₃ (244 mg, 749 μmol) in 1,4-dioxane (3 mL) and H₂O (0.3 mL) was degassed and purged with N₂ three times, and the mixture was stirred at 100° C. for 12 hr under N₂ atmosphere. The reaction mixture was diluted with H₂O (20 mL) and extracted with CH₂Cl₂ (10 mL×3). The combined organic layers were dried over Na₂SO₄, filtered and concentrated under reduced pressure. After purified by silica gel flash chromatography (Eluent of 0˜6% MeOH in CH₂Cl₂), tert-butyl (1-(4-(2-(2-aminopyridin-3-yl)-5-(3-chlorophenyl)-3H-imidazo[4,5-b]pyridin-3-yl)benzyl)piperidin-4-yl)carbamate (143 mg, yield: 40%) was obtained as brown yellow solid. MS: m/z=610.3 [M+H]⁺.

### Step 2: 3-(3-(4-((4-Aminopiperidin-1-yl)methyl)phenyl)-5-(3-chlorophenyl)-3H-imidazo[4,5-b]pyridin-2-yl)pyridin-2-amine

To a solution of tert-butyl (4-(4-(2-(2-aminopyridin-3-yl)-5-(3-chlorophenyl)-3H-imidazo[4,5-b]pyridin-3-yl)benzyl)cyclohexyl)carbamate (50 mg, 82 μmol) in CH₂Cl₂ (5 mL) was added HCl in 1,4-dioxane (4 M, 20.5 μL). The mixture was stirred at 25° C. for 1 hr. The mixture was concentrated under reduced pressure, 3-(3-(4-((4-aminopiperidin-1-yl)methyl)phenyl)-5-(3-chlorophenyl)-3H-imidazo[4,5-b]pyridin-2-yl)pyridin-2-amine (Intermediate 113, 10 mg, HCl salt, yield: 23%) was obtained as light-yellow solid. MS: m/z=510.3 [M+H]+. ¹H NMR (400 MHz, Methanol-d₄) δ 8.21 (d, J=8.4 Hz, 1H), 8.07-8.03 (m, 1H), 7.99-7.96 (m, 2H), 7.55 (d, J=8.4 Hz, 2H), 7.48-7.31 (m, 6H), 6.47 (dd, J=7.6, 4.8 Hz, 1H), 3.64 (s, 2H), 2.98-2.93 (m, 2H), 2.80-2.72 (m, 1H), 2.18-2.12 (m, 2H), 1.91-1.85 (m, 2H), 1.55-1.48 (m, 2H).

### Intermediate 114: 4-(2-(2-Aminopyridin-3-yl)-3-(4-(chloromethyl)phenyl)-3H-imidazo[4,5-b]pyridin-5-yl)-1-methyl-1,4-diazepan-2-one

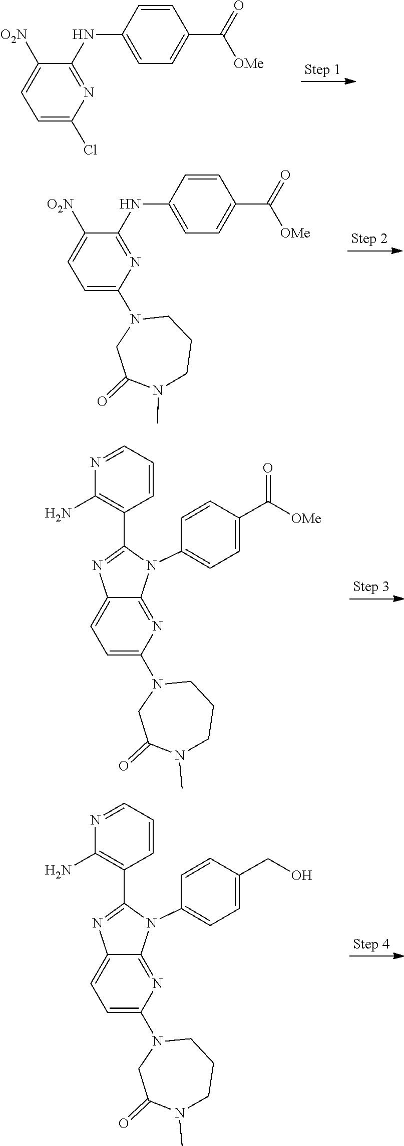

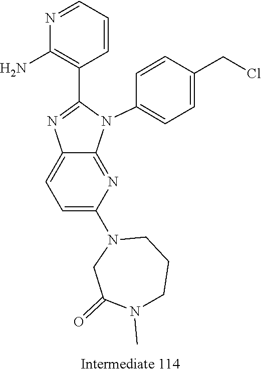 [SMILES: CN1CCCN(c2ccc3nc(-c4cccnc4N)n(-c4ccc(CCl)cc4)c3n2)CC1=O]

### Step 1: Methyl 4-((6-(4-methyl-3-oxo-1,4-diazepan-1-yl)-3-nitropyridin-2-yl)amino)benzoate

To a solution of methyl 4-((6-chloro-3-nitropyridin-2-yl)amino)benzoate (refer to Intermediate 13 for detail procedures, 5 g, 16.3 mmol), 1-methyl-1,4-diazepan-2-one (4.01 g HCl salt, 24.4 mmol) in ACN (50 mL) was added DIEA (6.30 g, 48.8 mmol). The mixture was stirred at 80° C. for 16 hr under N₂. Water (50 mL) was added, and the mixture was extracted with EtOAc (50 mL×2). The combined organic layers were filtered and concentrated. The filter cake was washed with ACN (10 mL). Methyl 4-((6-(4-methyl-3-oxo-1,4-diazepan-1-yl)-3-nitropyridin-2-yl)amino)benzoate (4.6 g, yield: 71%) was obtained as yellow solid, which was used in the next step without further purification. MS: m/z=400.0 [M+H]⁺. ¹H NMR (400 MHz, Dimethylsulfoxide-d₆) δ 10.90-10.65 (m, 1H), 8.28 (d, J=8.8 Hz, 1H), 8.12-7.98 (m, 1H), 7.95 (d, J=8.4 Hz, 2H), 7.88-7.75 (m, 1H), 6.65-6.37 (m, 1H), 4.63-4.30 (m, 2H), 3.84 (s, 3H) 3.62-3.47 (m, 2H), 3.40-3.25 (m, 2H), 2.83 (s, 3H), 1.97-1.793 (m, 2H).

### Step 2: Methyl 4-(2-(2-aminopyridin-3-yl)-5-(4-methyl-3-oxo-1,4-diazepan-1-yl)-3H-imidazo[4,5-b]pyridin-3-yl)benzoate

To a solution of methyl 4-((6-(4-methyl-3-oxo-1,4-diazepan-1-yl)-3-nitropyridin-2-yl)amino)benzoate (4.60 g, 11.5 mmol) in DMSO (15 mL) and MeOH (1.5 mL) were added 2-aminopyridine-3-carbaldehyde (1.55 g, 12.7 mmol) and Na₂S₂O₄ (6.02 g, 34.6 mmol). The mixture was stirred at 100° C. for 48 hr. Water (300 mL) was added to the mixture and the mixture was extracted with CH₂Cl₂ (50 mL×2). The combined organic layers were washed with brine (20 mL), dried over Na₂SO₄, filtered and concentrated. Methyl 4-(2-(2-aminopyridin-3-yl)-5-(4-methyl-3-oxo-1,4-diazepan-1-yl)-3H-imidazo[4,5-b]pyridin-3-yl)benzoate (4.7 g, yield: 87%) was obtained as yellow oil, which was used in next step without further purification. MS: m/z=472.3 [M+H]⁺. ¹H NMR (400 MHz, Dimethylsulfoxide-d₆) δ 8.06 (d, J=8.4 Hz, 2H), 8.02-7.97 (m, 2H), 7.58 (d, J=8.4 Hz, 2H), 7.45 (br s, 2H), 7.31 (d, J=7.8 Hz, 1H), 6.86 (d, J=8.8 Hz, 1H), 6.57 (dd, J=7.2, 5.6 Hz, 1H), 4.28 (s, 2H), 3.87 (s, 3H), 3.84-3.78 (m, 2H), 3.48-3.44 (m, 2H), 2.76 (s, 3H), 1.76-1.66 (m, 2H).

### Step 3: 4-(2-(2-Aminopyridin-3-yl)-3-(4-(hydroxymethyl)phenyl)-3H-imidazo[4,5-b]pyridin-5-yl)-1-methyl-1,4-diazepan-2-one

To a solution of methyl 4-(2-(2-aminopyridin-3-yl)-5-(4-methyl-3-oxo-1,4-diazepan-1-yl)-3H-imidazo[4,5-b]pyridin-3-yl)benzoate (1.0 g, 2.12 mmol) in THF (20 mL) was added LiAlH₄ (121 mg, 3.18 mmol) in portions under N₂ at 0° C. The mixture was stirred at 0° C. for 2 hr under N₂. The mixture was diluted with THF (20 mL). Na₂SO_{4·}10H₂O was added in portions until no bubbles were formed. The resulting mixture was stirred at 25° C. for 20 min and filtered. The filter cake was washed with THF (20 mL×2), the combined filtrate was concentrated. 4-(2-(2-Aminopyridin-3-yl)-3-(4-(hydroxymethyl)phenyl)-3H-imidazo[4,5-b]pyridin-5-yl)-1-methyl-1,4-diazepan-2-one (941 mg) as brown oli, which was used in the next step directly. MS: m/z=444.3 [M+H]⁺.

### Step 4: 4-(2-(2-Aminopyridin-3-yl)-3-(4-(chloromethyl)phenyl)-3H-imidazo[4,5-b]pyridin-5-yl)-1-methyl-1,4-diazepan-2-one

To a solution of 4-(2-(2-aminopyridin-3-yl)-3-(4-(hydroxymethyl)phenyl)-3H-imidazo[4,5-b]pyridin-5-yl)-1-methyl-1,4-diazepan-2-one (941 mg, 2.12 mmol) in CH₂Cl₂ (10 mL) was added SOCl₂ (757 mg, 6.36 mmol) at 25° C. The mixture was stirred at 40° C. for 1 hr under N₂. The mixture was concentrated. 4-(2-(2-Aminopyridin-3-yl)-3-(4-(chloromethyl)phenyl)-3H-imidazo[4,5-b]pyridin-5-yl)-1-methyl-1,4-diazepan-2-one (Intermediate 114, 980 mg) as brown solid, which was used in the next step directly. MS: m/z=462.2 [M+H]⁺.

### Intermediate 115: 3-(2-(2-Aminopyridin-3-yl)-3-(4-(chloromethyl)phenyl)-3H-imidazo[4,5-b]pyridin-5-yl)benzonitrile

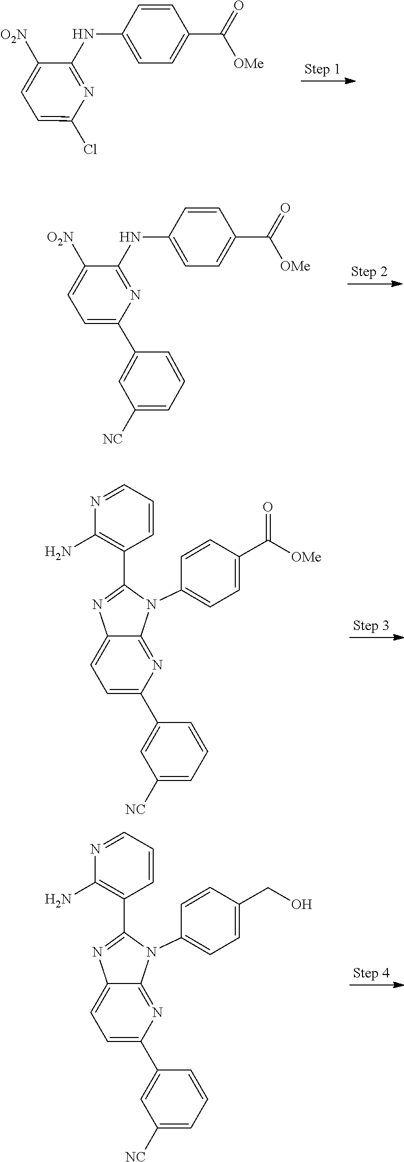

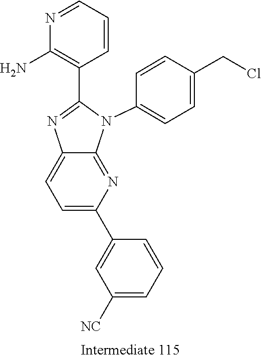 [SMILES: N#Cc1cccc(-c2ccc3nc(-c4cccnc4N)n(-c4ccc(CCl)cc4)c3n2)c1]

### Step 1: Methyl 4-((6-(3-cyanophenyl)-3-nitropyridin-2-yl)amino)benzoate

A mixture of methyl 4-((6-chloro-3-nitropyridin-2-yl)amino)benzoate (refer to Intermediate 13 for detail procedures, 25 g, 81.3 mmol), (3-cyanophenyl)boronic acid (13.1 g, 89.4 mmol), Cs₂CO₃ (79.4 g, 244 mmol), Pd(dppf)Cl₂ (5.95 g, 8.13 mmol) in 1,4-dioxane (250 mL) and H₂O (50 mL) was degassed and purged with N₂ three times, and then the mixture was stirred at 100° C. for 16 hr under N₂ atmosphere. The reaction mixture was diluted with H₂O (300 mL) and extracted with CH₂Cl₂ (200 mL×4). The combined organic layers were washed with brine (300 mL×3), dried over Na₂SO₄, filtered and concentrated under reduced pressure. After purified by silica gel flash chromatography (Eluent of 30˜100% EtOAc in petroleum ether), methyl 4-((6-(3-cyanophenyl)-3-nitropyridin-2-yl)amino)benzoate (18 g, yield: 60%) was obtained as a yellow solid. MS: m/z=374.9 [M+H]⁺. ¹H NMR (400 MHz, Dimethylsulfoxide-d₆) δ 10.23 (s, 1H), 8.74 (d, J=8.4 Hz, 1H), 8.04 (dd, J=7.6, 0.8 Hz, 1H), 7.96-7.92 (m, 4H), 7.92-7.85 (m, 2H), 7.73 (dt, J=7.2, 1.2 Hz, 1H), 7.50 (d, J=8.4 Hz, 1H), 3.83 (s, 3H).

### Step 2: Methyl 4-(2-(2-aminopyridin-3-yl)-5-(3-cyanophenyl)-3H-imidazo[4,5-h]pyridin-3-yl)benzoate

To a solution of methyl 4-((6-(3-cyanophenyl)-3-nitropyridin-2-yl)amino)benzoate (11.6 g, 31 mmol), 2-aminonicotinaldehyde (3.78 g, 31 mmol) in DMSO (120 mL) was added Na₂S₂O₄ (21.6 g, 124 mmol). The mixture was stirred at 100° C. for 16 hr. The reaction mixture was diluted with H₂O (300 mL) and extracted with CH₂Cl₂ (300 mL×5). The combined organic layers were washed with brine (250 mL×2), dried over Na₂SO₄, filtered and concentrated under reduced pressure. After purified by silica gel flash chromatography (Eluent of 30˜50% EtOAc in petroleum ether and 50% EtOAc in CH₂Cl₂), methyl 4-(2-(2-aminopyridin-3-yl)-5-(3-cyanophenyl)-3H-imidazo[4,5-b]pyridin-3-yl)benzoate (6 g, yield: 43%) was obtained as a black brown solid. MS: m/z=447.0 [M+H]⁺. ¹H NMR (400 MHz, Dimethylsulfoxide-d₆) δ 8.40 (d, J=8.4 Hz, 1H), 8.08-8.02 (m, 3H), 7.97-7.88 (m, 3H), 7.84-7.78 (m, 1H), 7.72-7.67 (m, 2H), 7.64-7.59 (m, 1H), 7.28 (dd, J=7.6, 1.6 Hz, 1H), 6.86 (br s, 2H), 6.47 (dd, J=8.0, 5.2 Hz, 1H), 3.88 (s, 3H).

### Step 3: 3-(2-(2-Aminopyridin-3-yl)-3-(4-(hydroxymethyl)phenyl)-3H-imidazo[4,5-b]pyridin-5-yl)benzonitrile

To a solution of methyl 4-(2-(2-aminopyridin-3-yl)-5-(3-cyanophenyl)-3H-imidazo[4,5-b]pyridin-3-yl)benzoate (1.5 g, 3.36 mmol) in THF (50 mL) was added LiAlH₄ (2.5 M, 2.69 mL) at 0° C., then the mixture was stirred at 25° C. for 1 hr. The reaction mixture was quenched with Na₂SO₄-10H₂O (2 g) at 0° C., and the mixture was filtered, the filter cake was washed by CH₂Cl₂ (30 mL×3), and the filtrate was concentrated under reduced pressure to give 3-(2-(2-aminopyridin-3-yl)-3-(4-(hydroxymethyl)phenyl)-3H-imidazo[4,5-b]pyridin-5-yl)benzonitrile (1.4 g, yield: 86%) as a yellow solid. MS: m/z=419.1 [M+H]⁺.

### Step 4: 3-(2-(2-Aminopyridin-3-yl)-3-(4-(chloromethyl)phenyl)-3H-imidazo[4,5-b]pyridin-5-yl)benzonitrile

To a solution of 3-(2-(2-aminopyridin-3-yl)-3-(4-(hydroxymethyl)phenyl)-3H-imidazo[4,5-b]pyridin-5-yl)benzonitrile (1.4 g, 3.35 mmol) in CH₂Cl₂ (20 mL) was added SOCl₂ (1.19 g, 10.1 mmol) at 25° C. The mixture was stirred at 40° C. for 1 hr. The reaction mixture was quenched with H₂O (20 mL) at 0° C., and the filtrate was concentrated under reduced pressure to give 3-(2-(2-aminopyridin-3-yl)-3-(4-(chloromethyl)phenyl)-3H-imidazo[4,5-b]pyridin-5-yl)benzonitrile (Intermediate 115, 1.6 g, yield: 98%) as a yellow solid. MS: m/z=437.0 [M+H]⁺.

### Intermediate 116: 4-(2-(2-Aminopyridin-3-yl)-3-(4-(chloromethyl)phenyl)-3H-imidazo[4,5-b]pyridin-5-yl)morpholin-3-one hydrochloride

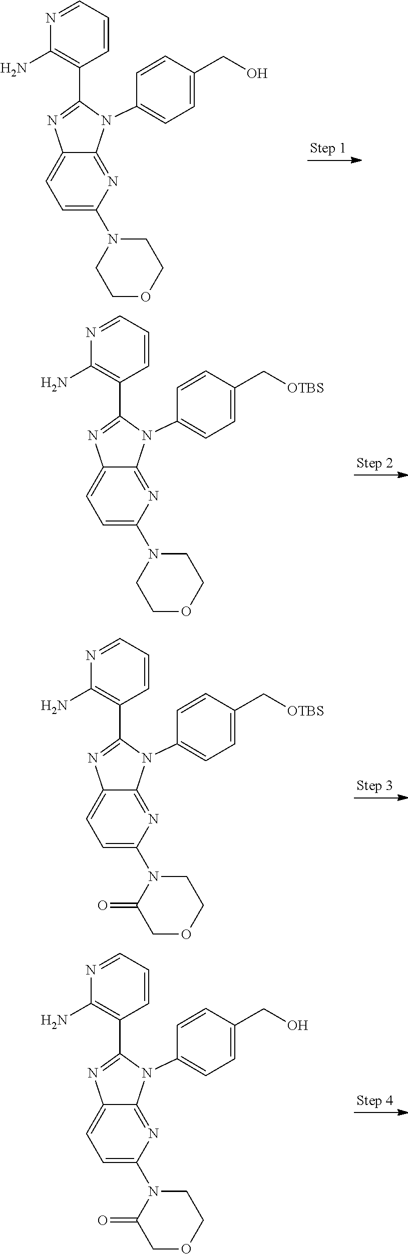

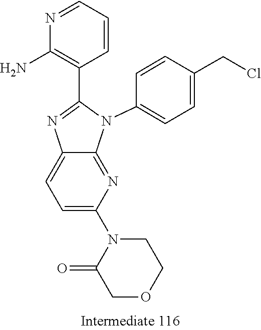 [SMILES: Nc1ncccc1-c1nc2ccc(N3CCOCC3=O)nc2n1-c1ccc(CCl)cc1]

### Step 1: 3-(3-(4-(((Tert-butyldimethylsilyl)oxy)methyl)phenyl)-5-morpholino-3H-imidazo[4,5-b]pyridin-2-yl)pyridin-2-amine

A mixture of (4-(2-(2-aminopyridin-3-yl)-5-morpholino-3H-imidazo[4,5-b]pyridin-3-yl)phenyl)methanol (refer to Intermediate 56 for detail procedures, 5.1 g, 13 mmol), TBSCl (3.8 g, 25 mmol) and imidazole (2.2 g, 32 mmol) in CH₂Cl₂ (70 mL) was degassed and purged with N₂ three times, and then the mixture was stirred at 25° C. for 2 hr under N₂ atmosphere. The reaction mixture was diluted with water (40 mL) and extracted with EtOAc (100 mL×2). The combined organic layers were washed with brine (30 mL), dried over Na₂SO₄, filtered and concentrated under reduced pressure. After purified by silica gel flash chromatography (Eluent of 0˜16%, EtOAc in petroleum ether), 3-(3-(4-(((tert-butyldimethylsilyl)oxy)methyl)phenyl)-5-morpholino-3H-imidazo[4,5-b]pyridin-2-yl)pyridin-2-amine (3.5 g, yield: 53%) was obtained as yellow solid. MS: m/z=517.6 [M+H]⁺. ¹H NMR (400 MHz, Chloroform-d) δ 8.00 (dd, J=5.2, 1.6 Hz, 1H), 7.90 (d, J=8.8 Hz, 1H), 7.43 (d, J=8.4 Hz, 2H), 7.34-7.28 (m, 2H), 7.04 (dd, J=7.6, 1.6 Hz, 1H), 6.69 (d, J=8.8 Hz, 1H), 6.60 (br s, 2H), 6.34 (dd, J=7.6, 4.8 Hz, 1H), 4.69 (s, 2H), 3.83-3.77 (m, 4H), 3.51-3.43 (m, 4H), 0.96 (s, 9H), 0.13 (s, 6H).

### Step 2: 4-(2-(2-Aminopyridin-3-yl)-3-(4-(((tert-butyldimethylsilyl)oxy)methyl)phenyl)-3H-imidazo[4,5-b]pyridin-5-yl)morpholin-3-one

To a solution of 3-(3-(4-(((tert-butyldimethylsilyl)oxy)methyl)phenyl)-5-morpholino-3H-imidazo[4,5-b]pyridin-2-yl)pyridin-2-amine (1.0 g, 1.9 mmol) in MeCN (30 mL) was added dropwise NaClO₂ (1.4 g, 15 mmol) in H₂O (5 mL) at 50° C. over 10 min. The resulting mixture was stirred at 50° C. for 12 hr under CO₂ atmosphere. The reaction mixture was concentrated under reduced pressure. After purified by silica gel flash chromatography (Eluent of 0˜100% EtOAc in petroleum ether), 4-(2-(2-aminopyridin-3-yl)-3-(4-(((tert-butyldimethylsilyl)oxy)methyl)phenyl)-3H-imidazo[4,5-b]pyridin-5-yl)morpholin-3-one (200 mg, yield: 16%) was obtained as a yellow solid. MS: m/z=531.1 [M+H]⁺. ¹H NMR (400 MHz, Chloroform-d) δ 8.11-8.02 (m, 3H), 7.48 (d, J=8.4 Hz, 2H), 7.31 (d, J=8.4 Hz, 2H), 7.14-7.11 (m, 1H), 6.99 (br s, 2H), 6.38 (dd, J=7.6, 5.2 Hz, 1H), 4.85 (s, 2H), 4.35 (s, 2H), 3.99 (br s, 4H), 0.97 (s, 9H), 0.14 (s, 6H).

### Step 3: 4-(2-(2-Aminopyridin-3-yl)-3-(4-(hydroxymethyl)phenyl)-3H-imidazo[4,5-b]pyridin-5-yl)morpholin-3-one

To a solution of 4-(2-(2-aminopyridin-3-yl)-3-(4-(((tert-butyldimethylsilyl)oxy)methyl)phenyl)-3H-imidazo[4,5-b]pyridin-5-yl)morpholin-3-one (200 mg, 377 μmol) in TBAF (1 M, 5 mL). The mixture was stirred at 25° C. for 1 hr. The reaction mixture was diluted with H₂O (30 mL) and extracted with EtOAc (30 mL×2). The combined organic layers were washed with brine (30 mL), dried over Na₂SO₄, filtered and concentrated under reduced pressure. The crude 4-(2-(2-aminopyridin-3-yl)-3-(4-(hydroxymethyl)phenyl)-3H-imidazo[4,5-b]pyridin-5-yl)morpholin-3-one (157 mg, yield: 100%) was used in the next step without further purification. MS: m/z=417.1 [M+H]⁺.

### Step 4: 4-(2-(2-Aminopyridin-3-yl)-3-(4-(chloromethyl)phenyl)-3H-imidazo[4,5-b]pyridin-5-yl)morpholin-3-one hydrochloride

To a solution of 4-(2-(2-aminopyridin-3-yl)-3-(4-(hydroxymethyl)phenyl)-3H-imidazo[4,5-b]pyridin-5-yl)morpholin-3-one (150 mg, 360 μmol) in CH₂Cl₂ (2 mL) was added SOCl₂ (129 mg, 1 mmol). The mixture was stirred at 25° C. for 1 hr. The reaction mixture was concentrated under reduced pressure. The crude 4-(2-(2-Aminopyridin-3-yl)-3-(4-(chloromethyl)phenyl)-3H-imidazo[4,5-b]pyridin-5-yl)morpholin-3-one hydrochloride (Intermediate 116, 170 mg HCl salt, yield: 100%) was used in the next step without further purification. MS: m/z=435.2 [M+H]⁺.

### Intermediate 117: 3-(5-Chloro-3-(4-(chloromethyl)phenyl)-3H-imidazo[4,5-b]pyridin-2-yl)pyridin-2-amine

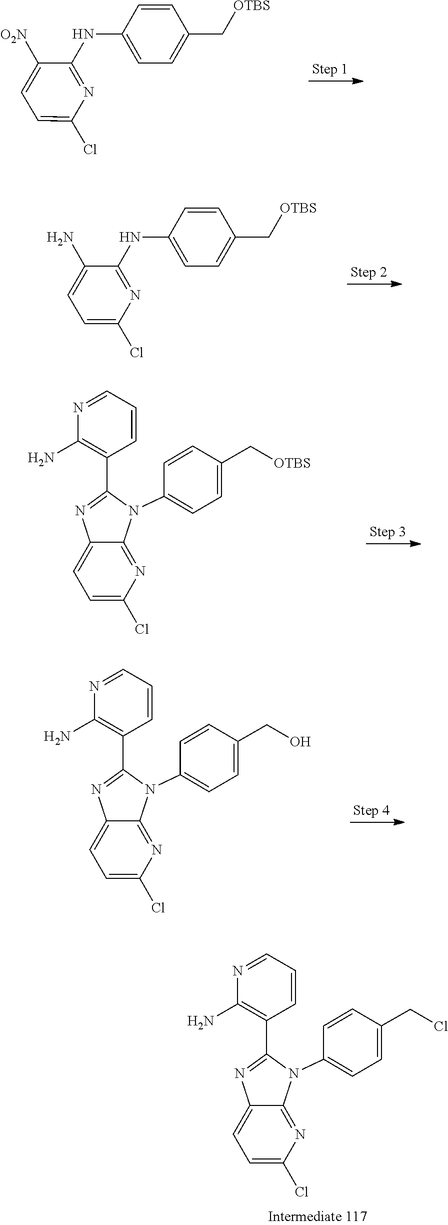

### Step 1: N²-(4-(((tert-butyldimethylsilyl)oxy)methyl)phenyl)-6-chloropyridine-2,3-diamine

To a solution of N-(4-(((tert-butyldimethylsilyl)oxy)methyl)phenyl)-6-chloro-3-nitropyridin-2-amine (refer to Intermediate 105 for detail procedures, 5 g, 12.7 mmol) in EtOH (50 mL) and H₂O (10 mL) was added Fe (1.42 g, 25.4 mmol) and NH₄Cl (3.39 g, 63.5 mmol). The mixture was degassed and purged with N₂ three times and stirred at 80° C. for 2 hr under N₂. The reaction mixture was filtered and concentrated under reduced pressure to give N²-(4-(((tert-butyldimethylsilyl)oxy)methyl)phenyl)-6-chloropyridine-2,3-diamine (4.62 g, crude) as a black solid, which was used in the next step directly. MS: m/z=364.2 [M+H]⁺.

### Step 2: 3-(3-(4-(((tert-butyldimethylsilyl)oxy)methyl)phenyl)-5-chloro-3H-imidazo[4,5-b]pyridin-2-yl)pyridin-2-amine

To a solution of N²-(4-(((tert-butyldimethylsilyl)oxy)methyl)phenyl)-6-chloropyridine-2,3-diamine (13.9 g, 38.1 mmol), 2-aminonicotinaldehyde (4.65 g, 38.1 mmol) in EtOH (150 mL) was added InCl₃ (842 mg, 3.81 mmol). The mixture was degassed and purged with N₂ three times and stirred at 80° C. for 2 hr under N₂. Then MnO₂ (19.9 g, 228 mmol) was added. The mixture was degassed and purged with N₂ three times and stirred at 80° C. for 2 hr under N₂. The reaction mixture was filtered and concentrated under reduced pressure. After purified by silica gel flash chromatography (Eluent of 1˜50% EtOAc in petroleum ether), 3-(3-(4-(((tert-butyldimethylsilyl)oxy)methyl)phenyl)-5-chloro-311-imidazo[4,5-h]pyridin-2-yl)pyridin-2-amine (6.02 g, 11.8 mmol) was obtained as a gray solid. MS: m/z=466.3 [M+H]+. ¹H NMR (400 MHz, Chloroform-d) δ 8.06-8.00 (m, 2H), 7.49 (d, J=8.0 Hz, 2H), 7.31 (dd, J=1.8, 8.4 Hz, 3H), 7.09 (dd, J=7.6, 1.2 Hz, 1H), 6.80 (br s, 2H), 6.36 (dd, J=7.6, 5.2 Hz, 1H), 4.83 (s, 2H), 0.97 (s, 9H), 0.14 (s, 6H).

### Step 3: (4-(2-(2-Aminopyridin-3-yl)-5-chloro-3H-imidazo[4,5-b]pyridin-3-yl)phenyl)methanol

To a solution of 3-(3-(4-(((tert-butyldimethylsilyl)oxy)methyl)phenyl)-5-chloro-3H-imidazo[4,5-b]pyridin-2-yl)pyridin-2-amine (500 mg, 1.07 mmol) in CH₂Cl₂ (5 mL) was added TBAF (1 M, 1.61 mL). The mixture was degassed and purged with N₂ three times and stirred at 25° C. for 2 hr under N₂. The reaction mixture was quenched with Sat.NH₄Cl (20 mL) at 25° C., and then extracted with CH₂Cl₂ (10 mL×3). The combined organic layers were washed with brine (20 mL×2), dried over Na₂SO₄, filtered and concentrated under reduced pressure. (4-(2-(2-aminopyridin-3-yl)-5-chloro-3H-imidazo[4,5-b]pyridin-3-yl)phenyl)methanol (377 mg, crude) was obtained as a yellow solid. MS: m/z=352.2 [M+H]⁺.

### Step 4: 3-(5-Chloro-3-(4-(chloromethyl)phenyl)-3H-imidazo[4,5-b]pyridin-2-yl)pyridin-2-amine

To a solution of (4-(2-(2-aminopyridin-3-yl)-5-chloro-3H-imidazo[4,5-b]pyridin-3-yl)phenyl)methanol (377 mg, 1.07 mmol) in CH₂Cl₂ (5 mL) was added SOCl₂ (1.28 g, 10.7 mmol). The mixture was degassed and purged with N₂ three times and stirred at 25° C. for 4 hr under N₂. The reaction mixture was concentrated under reduced pressure to give 3-(5-chloro-3-(4-(chloromethyl)phenyl)-3H-imidazo[4,5-b]pyridin-2-yl)pyridin-2-amine (Intermediate 117, 430 mg, crude) as a gray solid. MS: m/z=370.1 [M+H]⁺.

### Intermediate 118: 3-(3-(4-(Chloromethyl)phenyl)-5-cyclopentyl-3H-imidazo[4,5-b]pyridin-2-yl)pyridin-2-amine

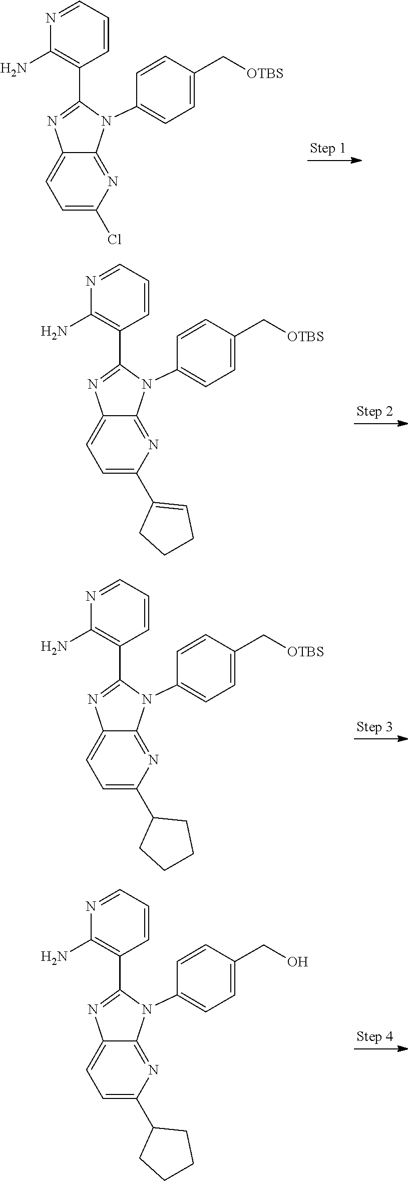

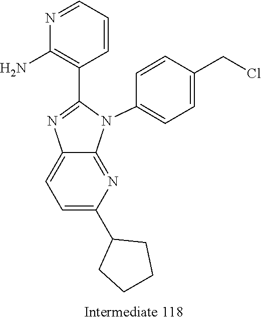 [SMILES: Nc1ncccc1-c1nc2ccc(C3CCCC3)nc2n1-c1ccc(CCl)cc1]

### Step 1: 3-(3-(4-(((Tert-butyldimethylsilyl)oxy)methyl)phenyl)-5-(cyclopent-1-en-1-yl)-3H-imidazo[4,5-b]pyridin-2-yl)pyridin-2-amine

A mixture of 3-(3-(4-(((tert-butyldimethylsilyl)oxy)methyl)phenyl)-5-chloro-3H-imidazo[4,5-b]pyridin-2-yl)pyridin-2-amine (refer to Intermediate 117 for detail procedures, 1 g, 2.15 mmol), cyclopenten-1-ylboronic acid (288 mg, 2.57 mmol), Cs₂CO₃ (2.1 g, 6.4 mmol), Pd(dppf)Cl₂ (157 mg, 215 μmol) in 1,4-dioxane (10 mL) and H₂O (2 mL) was degassed and purged with N₂ three times, then the mixture was stirred at 100° C. for 16 hr under N₂ atmosphere. The reaction mixture was poured into H₂O (15 mL), extracted with EtOAc (15 mL×3). The combined organic layers were dried over anhydrous Na₂SO₄, filtered and concentrated under reduced pressure. After purified by silica gel flash chromatography (Eluent of 0˜35% EtOAc in petroleum ether), 3-(3-(4-(((tert-butyldimethylsilyl)oxy)methyl)phenyl)-5-(cyclopent-1-en-1-yl)-3H-imidazo[4,5-b]pyridin-2-yl)pyridin-2-amine (930 mg, yield: 84%) was obtained as a gray solid. MS: m/z=498.1 [M+H]⁺. ¹H NMR (400 MHz, Chloroform-d) δ 8.04 (dd, J=4.8, 1.6 Hz, 1H), 7.98 (d, J=8.4 Hz, 1H), 7.50-7.32 (m, 5H), 7.12 (dd, J=7.6, 1.6 Hz, 1H), 6.67 (br s, 2H), 6.56 (t, J=2.0 Hz, 1H), 6.36 (dd, J=7.6, 5.2 Hz, 1H), 4.84 (s, 2H), 2.88-2.72 (m, 2H), 2.59-2.50 (m, 2H), 2.01 (d, J=7.6 Hz, 1H), 1.86-1.74 (m, 1H), 0.97 (s, 9H), 0.14 (s, 6H)

### Step 2: 3-(3-(4-(((Tert-butyldimethylsilyl)oxy)methyl)phenyl)-5-cyclopentyl-3H-imidazo[4,5-b]pyridin-2-yl)pyridin-2-amine

To a solution of 3-(3-(4-(((tert-butyldimethylsilyl)oxy)methyl)phenyl)-5-(cyclopent-1-en-1-yl)-3H-imidazo[4,5-b]pyridin-2-yl)pyridin-2-amine (160 mg, 322 μmol) in MeOH (10 mL) was added Pd/C (230 mg, 216 μmol, 10% purity) under N₂. The suspension was degassed under reduced pressure and purged with H₂ several times. The mixture was stirred under H₂ (15 psi) at 25° C. for 4 hr. The reaction mixture was filtered and concentrated under reduced pressure. The crude 3-(3-(4-(((tert-butyldimethylsilyl)oxy)methyl)phenyl)-5-cyclopentyl-3H-imidazo[4,5-b]pyridin-2-yl)pyridin-2-amine (150 mg, yield: 94%) was obtained as a yellow solid. MS: m/z=500.3 [M+H]⁺. ¹H NMR (400 MHz, Dimethylsulfoxide-d₆) δ 8.07 (d, J=8.0 Hz, 1H), 7.97 (dd, J=4.4, 1.6 Hz, 1H), 7.46-7.43 (m, 2H), 7.40-7.36 (m, 2H), 7.29 (d, J=8.4 Hz, 1H), 7.13 (dd, J=7.2, 1.6 Hz, 1H), 6.93 (br s, 2H), 6.36 (dd, J=7.6, 4.8 Hz, 1H), 4.80 (s, 2H), 4.10-4.08 (m, 1H), 1.98-1.94 (m, 2H), 1.75-1.67 (m, 4H), 1.64-1.58 (m, 2H), 0.92 (s, 9H), 0.10 (s, 6H).

### Step 3: (4-(2-(2-Aminopyridin-3-yl)-5-cyclopentyl-3H-imidazo[4,5-b]pyridin-3-yl)phenyl)methanol

To a solution of 3-(3-(4-(((tert-butyldimethylsilyl)oxy)methyl)phenyl)-5-cyclopentyl-3H-imidazo[4,5-b]pyridin-2-yl)pyridin-2-amine (150 mg, 300 μmol) in THF (2 mL) was added TBAF (157 mg, 600 μmol). The mixture was stirred at 25° C. for 0.3 hr. The reaction mixture was poured into H₂O (15 mL), extracted with EtOAc (15 mL×3). The combined organic layers were dried over anhydrous Na₂SO₄, filtered and concentrated under reduced pressure, (4-(2-(2-aminopyridin-3-yl)-5-cyclopentyl-3H-imidazo[4,5-b]pyridin-3-yl)phenyl)methanol (110 mg, crude) as a yellow oil. MS: m/z=386.1 [M+H]⁺.

### Step 4: 3-(3-(4-(Chloromethyl)phenyl)-5-cyclopentyl-3H-imidazo[4,5-b]pyridin-2-yl)pyridin-2-amine

To a solution of (4-(2-(2-aminopyridin-3-yl)-5-cyclopentyl-3H-imidazo[4,5-b]pyridin-3-yl)phenyl)methanol (110 mg, 285 μmol) in CH₂Cl₂ (2 mL) was added SOCl₂ (68 mg, 571 μmol). The mixture was stirred at 40° C. for 0.5 hr. The mixture was concentrated under reduced pressure to give the crude 3-(3-(4-(chloromethyl)phenyl)-5-cyclopentyl-3H-imidazo[4,5-b]pyridin-2-yl)pyridin-2-amine (Intermediate 118, 100 mg) as a yellow solid. MS: m/z=404.1 [M+H]⁺.

### Intermediate 119: 2-(2-Aminopyridin-3-yl)-3-(4-(chloromethyl)phenyl)-N,N-dimethyl-3H-imidazo[4,5-b]pyridin-5-amine

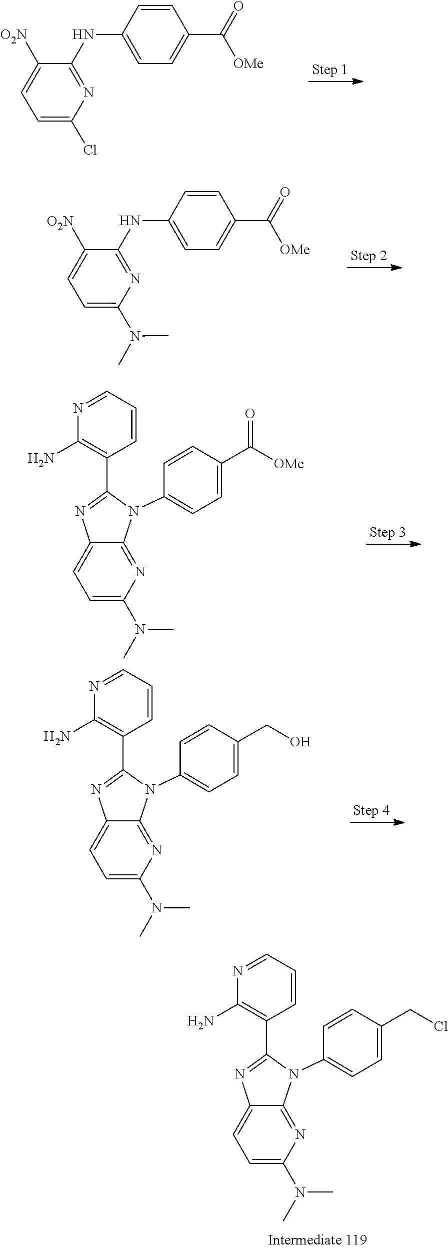

### Step 1: Methyl 4-((6-(dimethylamino)-3-nitropyridin-2-yl)amino)benzoate

To a solution of methyl 4-((6-chloro-3-nitropyridin-2-yl)amino)benzoate (refer to Intermediate 13 for detail procedures, 5 g, 16.3 mmol) in MeCN (50 mL) were added DIEA (10.5 g, 81.3 mmol) and dimethylamine (1.33 g, 1.49 mmol). The mixture was stirred at 90° C. for 16 hr. The reaction mixture was quenched with H₂O (50 mL) at 25° C., and then extracted with CH₂Cl₂ (50 mL×3). The combined organic layers were washed with brine (50 mL), dried over Na₂SO₄, filtered and concentrated under reduced pressure. The crude product was triturated with MeCN at 25° C. for 30 min, methyl 4-((6-(dimethylamino)-3-nitropyridin-2-yl)amino)benzoate (4.5 g, yield: 87%) was obtained as a yellow solid. MS: m/z=317.0 [M+H]˜. ¹H NMR (400 MHz, Dimethylsulfoxide-d₆) δ 10.86 (s, 1H), 8.22 (d, J=9.6 Hz, 1H), 7.95 (d, J=8.8 Hz, 1H), 7.89 (d, 1=8.8 Hz, 1H), 6.42 (d, J=9.6 Hz, 1H), 3.83 (s, 3H), 3.19 (s, 6H).

### Step 2: Methyl 4-(2-(2-aminopyridin-3-yl)-5-(dimethylamino)-3H-imidazo[4,5-b]pyridin-3-yl)benzoate

To a solution of methyl 4-((6-(dimethylamino)-3-nitropyridin-2-yl)amino)benzoate (4.5 g, 14.2 mmol) in DMSO (50 mL) were added Na₂S₂O₄ (9.91 g, 56.9 mmol, 87% purity) and 2-aminonicotinaldehyde (1.91 mg, 15.7 mmol). The mixture was stirred at 100° C. for 16 hr. The reaction mixture was quenched with H₂O (100 mL) at 25° C., and then extracted with CH₂Cl₂ (100 mL×3). The combined organic layers were washed with brine (50 mL), dried over Na₂SO₄, filtered and concentrated under reduced pressure. After triturated with CH₂Cl₂: EtOAc (1:10) at 25° C. for 30 min, methyl 4-(2-(2-aminopyridin-3-yl)-5-(dimethylamino)-3H-imidazo[4,5-b]pyridin-3-yl)benzoate (6 g, yield: 95%) was obtained as a brown solid. MS: m/z=389.1 [M+H]⁺. ¹H NMR (400 MHz, Dimethylsulfoxide-d₆) δ 8.07 (d, J=8.4 Hz, 2H), 7.97 (dd, J=5.2, 1.6 Hz, 1H), 7.94 (d, J=8.8 Hz, 1H), 7.56 (d, J=8.4 Hz, 2H), 7.36 (br s, 2H), 7.19 (d, J=7.6 Hz, 1H), 6.73 (d, J=8.8 Hz, 1H), 6.49 (dd, J=7.6, 5.2 Hz, 1H), 3.88 (s, 3H), 3.01 (s, 3H), 2.54 (s, 3H).

### Step 3: (4-(2-(2-Aminopyridin-3-yl)-5-(dimethylamino)-3H-imidazo[4,5-b]pyridin-3-yl)phenyl)methanol

To a solution of methyl 4-(2-(2-aminopyridin-3-yl)-5-(dimethylamino)-3H-imidazo[4,5-b]pyridin-3-yl)benzoate (500 mg, 1.29 mmol) in THF (5 mL) was added LiAlH₄ (97.7 mg, 2.5 mmol) at 0° C. The resulting mixture was stirred at 25° C. for 1 hr. The reaction mixture was filtered, and concentrated to give (4-(2-(2-aminopyridin-3-yl)-5-(dimethylamino)-3H-imidazo[4,5-b]pyridin-3-yl)phenyl)methanol (350 mg, yield: 75%) as a yellow solid. MS: m/z=361.1 [M+H]⁺.

### Step 4: 2-(2-Aminopyridin-3-yl)-3-(4-(chloromethyl)phenyl)-N,N-dimethyl-3H-imidazo[4,5-b]pyridin-5-amine

To a solution of (4-(2-(2-aminopyridin-3-yl)-5-(dimethylamino)-3H-imidazo[4,5-b]pyridin-3-yl)phenyl)methanol (350 mg, 971 μmol) in CH₂Cl₂ (5 mL) was added SOCl₂ (347 mg, 2.91 mmol). The mixture was stirred at 40° C. for 0.5 hr. The reaction was concentrated under reduced pressure to give 2-(2-aminopyridin-3-yl)-3-(4-(chloromethyl)phenyl)-N,N-dimethyl-3H-imidazo[4,5-b]pyridin-5-amine (Intermediate 119, 430 mg, yield: 82%) as a yellow solid. MS: m/z=379.0 [M+H]⁺.

### Intermediate 120: 4-(Methyl(piperidin-4-yl)amino)pyrimidine-2-carbonitrile

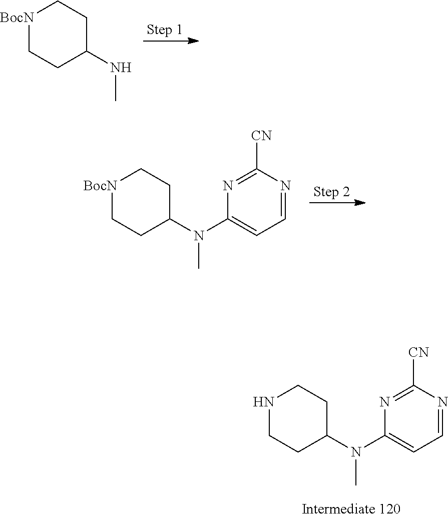 [SMILES: CNC1CCN(C(=O)OC(C)(C)C)CC1 ; CN(c1ccnc(C#N)n1)C1CCN(C(=O)OC(C)(C)C)CC1 ; CN(c1ccnc(C#N)n1)C1CCNCC1]

### Step 1: tert-Butyl 4-((2-cyanopyrimidin-4-yl)(methyl)amino)piperidine-1-carboxylate

To a solution of tert-butyl 4-(methylamino)piperidine-1-carboxylate (500 mg, 2.3 mmol) and 4-chloropyrimidine-2-carbonitrile (326 mg, 2.3 mmol) in DMF (5 mL) were added NaI (35 mg, 233 μmol) and K₂CO₃ (645 mg, 4.7 mmol). The mixture was stirred at 80° C. for 2 hr. The reaction mixture was quenched with H₂O (10 mL) at 25° C., and extracted with EtOAc (10 mL×2). The combined organic layers were washed with brine (10 mL), dried over Na₂SO₄, filtered and concentrated under reduced pressure. After purified by silica gel flash chromatography (Eluent of 0˜50%, EtOAc in petroleum ether), tert-butyl 4-((2-cyanopyrimidin-4-yl)(methyl)amino)piperidine-1-carboxylate (700 mg, yield: 95%) was obtained as a light yellow solid. MS: m/z=318.2 [M+H]⁺. ¹H NMR (400 MHz, Chloroform-d) δ 8.19 (d, J=6.4 Hz, 1H), 6.51 (d, J=6.0 Hz, 1H), 5.29-4.62 (m, 1H), 4.25 (s, 2H), 2.93-2.81 (m, 5H), 1.68-1.62 (m, 4H), 1.47 (s, 9H).

### Step 2: 4-(Methyl(piperidin-4-yl)amino)pyrimidine-2-carbonitrile

To a solution of tert-butyl 4-((2-cyanopyrimidin-4-yl)(methyl)amino)piperidine-1-carboxylate (150 mg, 473 μmol) in CH₂Cl₂ (3 mL) was added TFA (1.5 g, 13 mmol). The mixture was stirred at 25° C. for 0.5 hr. The reaction mixture was concentrated under reduced pressure to give 4-(methyl(piperidin-4-yl)amino)pyrimidine-2-carbonitrile (Intermediate 120, 156 mg) as a light yellow oil, which was used in the next step without further purification. MS: m/z=218.0 [M+H]⁺.

### Intermediate 121: 4-((4-Methylpiperidin-4-yl)amino)pyrimidine-2-carbonitrile

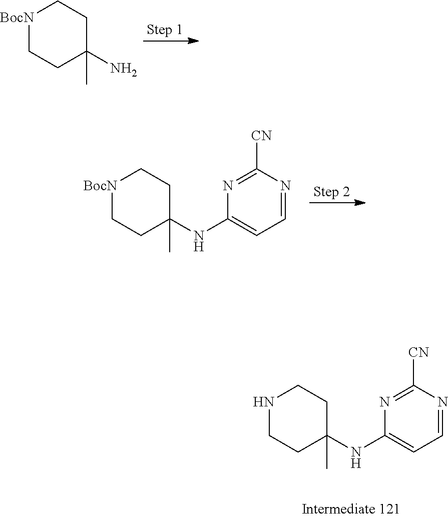 [SMILES: CC1(N)CCN(C(=O)OC(C)(C)C)CC1 ; CC1(Nc2ccnc(C#N)n2)CCN(C(=O)OC(C)(C)C)CC1 ; CC1(Nc2ccnc(C#N)n2)CCNCC1]

### Step 1: tert-Butyl 4-((2-cyanopyrimidin-4-yl)amino)-4-methylpiperidine-1-carboxylate

To a solution of tert-butyl 4-amino-4-methylpiperidine-1-carboxylate (500 mg, 2.3 mmol) and 4-chloropyrimidine-2-carbonitrile (326 mg, 2.3 mmol) in DMF (5 mL) were added NaI (35 mg, 233 μmol) and K₂CO₃ (645 mg, 4.7 mmol). The mixture was stirred at 80° C. for 2 hr. The reaction mixture was quenched with H₂O (10 mL) at 25° C., and extracted with EtOAc (10 mL×2). The combined organic layers were washed with brine (10 mL), dried over Na₂SO₄, filtered and concentrated under reduced pressure. After purified by silica gel flash chromatography (Eluent of 0˜50% EtOAc in petroleum ether), tert-butyl 4-((2-cyanopyrimidin-4-yl)amino)-4-methylpiperidine-1-carboxylate (700 mg, yield: 95%) was obtained as a light-yellow oil. MS: m/z=318.1 [M+H]⁺. ¹H NMR (400 MHz, Chloroform-d) δ 11 (d, J=6.0 Hz, 1H), 6.47 (d, J=6.0 Hz, 1H), 5.10 (br s, 1H), 3.66-3.59 (m, 2H), 3.26-3.19 (m, 2H), 1.74-1.68 (m, 2H), 1.51 (s, 3H), 1.45 (s, 9H), 1.44-1.43 (m, 2H).

### Step 2: 4-((4-Methylpiperidin-4-yl)amino)pyrimidine-2-carbonitrile

To a solution of tert-butyl 4-((2-cyanopyrimidin-4-yl)amino)-4-methylpiperidine-1-carboxylate (150 mg, 473 μmol) in CH₂Cl₂ (5 mL) was added TFA (1.5 g, 13 mmol). The mixture was stirred at 25° C. for 0.5 hr. The reaction mixture was concentrated under reduced pressure to give 4-((4-methylpiperidin-4-yl)amino)pyrimidine-2-carbonitrile (Intermediate 121, 100 mg) as a light-yellow oil, which was used directly in the next step. MS: m/z=218.0 [M+H]⁺

### Intermediate 122: 3-(3-(4-(Chloromethyl)phenyl)-5-(3-methoxyphenyl)-3H-imidazo[4,5-b]pyridin-2-yl)pyridin-2-amine

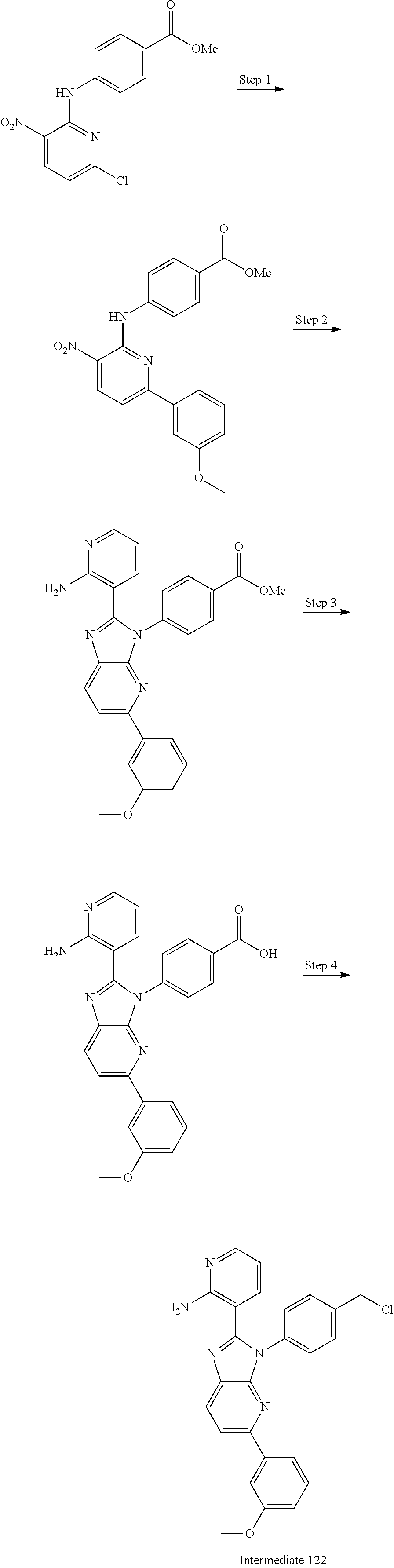 [SMILES: COC(=O)c1ccc(Nc2nc(Cl)ccc2[N+](=O)[O-])cc1 ; COC(=O)c1ccc(Nc2nc(-c3cccc(OC)c3)ccc2[N+](=O)[O-])cc1 ; COC(=O)c1ccc(-n2c(-c3cccnc3N)nc3ccc(-c4cccc(OC)c4)nc32)cc1 ; COc1cccc(-c2ccc3nc(-c4cccnc4N)n(-c4ccc(C(=O)O)cc4)c3n2)c1 ; COc1cccc(-c2ccc3nc(-c4cccnc4N)n(-c4ccc(CCl)cc4)c3n2)c1]

### Step 1: Methyl 4-((6-(3-methoxyphenyl)-3-nitropyridin-2-yl)amino)benzoate

To a solution of methyl 4-((6-chloro-3-nitropyridin-2-yl)amino)benzoate (refer to Intermediate 13 for detail procedures, 5 g, 16 mmol) and 2-(3-methoxyphenyl)-4,4,5,5-tetramethyl-1,3,2-dioxaborolane (3.8 g, 16 mmol) in 1,4-dioxane (50 mL) and H₂O (5 mL) were added Cs₂CO₃ (16 g, 49 mmol) and Pd(dppf)Cl₂ (1.2 g, 1.6 mmol) at 25° C. The mixture was stirred at 80° C. for 12 hr. The reaction mixture was poured into water (900 mL), extracted with EtOAc (900 mL×2). The combined organic layers were washed with brine (500 ml) and dried over anhydrous Na₂SO₄, filtered and concentrated to dryness. After purified by silica gel flash chromatography (Eluent of 0˜100% EtOAc in petroleum ether), methyl 4-((6-(3-methoxyphenyl)-3-nitropyridin-2-yl)amino)benzoate (4.93 g, yield: 80%) was obtained as a yellow solid. MS: m/z=380.0 [M+H]⁺.

### Step 2: Methyl 4-(2-(2-aminopyridin-3-yl)-5-(3-methoxyphenyl)-3H-imidazo[4,5-b]pyridin-3-yl)benzoate

A mixture of methyl 4-((6-(3-methoxyphenyl)-3-nitropyridin-2-yl)amino)benzoate (6.2 g, 16 mmol) and Na₂S₂O₄ (2.8 g, 16 mmol) in DMSO (60 mL) was degassed and purged with N₂ three times, and then the mixture was stirred at 100° C. for 16 hr under N₂ atmosphere. The reaction mixture was diluted with H₂O (600 mL) and extracted with CH₂Cl₂ (900 mL). The combined organic layers were washed with brine (300 mL), dried over Na₂SO₄, filtered and concentrated under reduced pressure. After purified by silica gel flash chromatography (Eluent of 0˜80%, EtOAc in petroleum ether), methyl 4-(2-(2-aminopyridin-3-yl)-5-(3-methoxyphenyl)-3H-imidazo[4,5-b]pyridin-3-yl)benzoate (2.0 g, yield: 27%) was obtained as a brown solid. MS: m/z=452.1 [M+H]⁺. ¹H NMR (400 MHz, Chloroform-d) δ 8.20 (d, J=8.4 Hz, 2H), 8.13 (d, J=8.4 Hz, 1H), 8.08 (dd, J=4.8, 1.6 Hz, 1H), 7.82 (d, J=8.4 Hz, 1H), 7.62-7.53 (m, 4H), 7.39-7.33 (m, 1H), 7.07 (dd, J=8.0, 1.6 Hz, 1H), 6.97-6.90 (m, 1H), 6.68 (br s, 2H), 6.40 (dd, J=7.6, 4.8 Hz, 1H), 3.97 (s, 3H), 3.86 (s, 3H).

### Step 3: (4-(2-(2-Aminopyridin-3-yl)-5-(3-methoxyphenyl)-3H-imidazo[4,5-b]pyridin-3-yl)phenyl)methanol

To a solution of methyl 4-(2-(2-aminopyridin-3-yl)-5-(3-methoxyphenyl)-3H-imidazo[4,5-b]pyridin-3-yl)benzoate (1.0 g, 2 mmol) in THF (10 mL) was added LiAlH₄ (2.5 M, 1.8 mL) at 0° C. The mixture was stirred at 25° C. for 1 hr. The mixture was added Na₂SO₄·10H₂O (1 g) slowly at 0° C., then stirred at 25° C. for 20 min. The mixture was filtered and conentrated under reduced pressure to give (4-(2-(2-aminopyridin-3-yl)-5-(3-methoxyphenyl)-3H-imidazo[4,5-b]pyridin-3-yl)phenyl)methanol (800 mg as a yellow solid.

### Step 4: 3-(3-(4-(Chloromethyl)phenyl)-5-(3-methoxyphenyl)-3H-imidazo[4,5-b]pyridin-2-yl)pyridin-2-amine

To a solution of (4-(2-(2-aminopyridin-3-yl)-5-(3-methoxyphenyl)-3H-imidazo[4,5-b]pyridin-3-yl)phenyl)methanol (800 mg, 1.9 mmol) in CH₂Cl₂ (5 mL) was added SOCl₂ (450 mg, 3.8 mmol). The mixture was stirred at 40° C. for 1 hr. The mixture was concentrated under reduced pressure, 3-(3-(4-(chloromethyl)phenyl)-5-(3-methoxyphenyl)-3H-imidazo[4,5-b]pyridin-2-yl)pyridin-2-amine (Intermediate 122, 800 mg, crude) was obtained as a yellow solid. MS: m/z=442.1 [M+H]⁺.

### Intermediate 123: 3-(5-(Tert-butyl)-3-(4-(chloromethyl)phenyl)-3H-imidazo[4,5-b]pyridin-2-yl)pyridin-2-amine

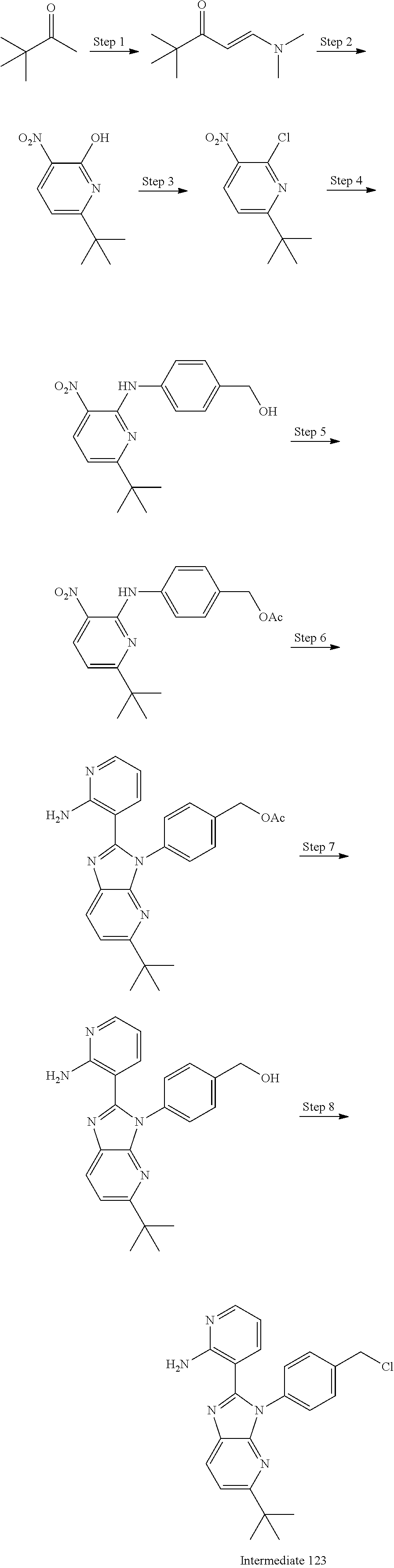

### Step 1: (E)-1-(dimethylamino)-4,4-dimethylpent-1-en-3-one

To a solution of 3,3-dimethylbutan-2-one (10 g, 100 mmol) in DMF (100 mL) was added DMF-DMA (23.8 g, 200 mmol) and CH₃ONa (539 mg, 9.98 mmol). The mixture was stirred at 100° C. for 16 hr. The mixture was diluted with H₂O (500 mL) and extracted with CH₂Cl₂ (500 mL×2). The combined organic layers were washed with brine (500 mL), dried over Na₂SO₄, filtered and concentrated under reduced pressure to give (E)-1-(dimethylamino)-4,4-dimethylpent-1-en-3-one (10.6 g, yield: 68%) as a brown oil. MS: m/z=156.2 [M+H]r.

### Step 2: 6-(Tert-butyl)-3-nitropyridin-2-ol

To a solution of (E)-1-(dimethylamino)-4,4-dimethylpent-1-en-3-one (10.6 g, 68.3 mmol) in H₂O (100 mL) were added piperidinium acetate (5 g, 34 mmol) and 2-nitroacetamide (10.6 g, 102 mmol). The mixture was stirred at 25° C. for 16 hr. The reaction mixture was filtered and the filter cake was dried to give 6-(tert-butyl)-3-nitropyridin-2-ol (3.1 g, yield: 23%) as a light-yellow solid. MS: m/z=197.1 [M+H]⁺. ¹H NMR (400 MHz, Chloroform-d) 8.41 (d, J=8.0 Hz, 1H), 6.34 (d, J=7.6 Hz, 1H), 1.44 (s, 9H).

### Step 3: 6-(Tert-butyl)-3-nitropyridin-2-ol

A solution of 6-(tert-butyl)-3-nitropyridin-2-ol (3.1 g, 15.8 mmol) in POCl₃ (5 mL) was stirred at 100° C. for 16 hr. The reaction mixture was concentrated under reduced pressure to remove POCl₃. The reaction mixture was poured into H₂O (20 mL), extracted with CH₂Cl₂ (60 mL). The organic phase was dried over anhydrous Na₂SO₄, filtered and concentrated under reduced pressure. 6-(ert-Butyl)-3-nitropyridin-2-ol (2.3 g, crude) was obtained as a black brown oil. MS: m/z=214.9 [M+Na]˜.

### Step 4: (4-((6-(tert-Butyl)-3-nitropyridin-2-yl)amino)phenyl)methanol

To a solution of 6-(tert-butyl)-3-nitropyridin-2-ol (1 g, 4.7 mmol) in DMSO (10 mL) were added DIEA (1.8 g, 14 mmol) and (4-aminophenyl)methanol (574 mg, 4.7 mmol). The mixture was stirred at 100° C. for 16 hr. The reaction mixture was poured into H₂O (50 mL) and was extracted with EtOAc (300 mL). The organic phase was dried over anhydrous Na₂SO₄, filtered and concentrated under reduced pressure. to give (4-((6-(tert-butyl)-3-nitropyridin-2-yl)amino)phenyl)methanol (1.5 g) as a yellow oil. MS: m/z=302.1 [M+H]⁺.

### Step 5: 4-((6-(Tert-butyl)-3-nitropyridin-2-yl)amino)benzyl acetate

To a solution of (4-((6-(tert-butyl)-3-nitropyridin-2-yl)amino)phenyl)methanol (1.5 g, 5 mmol) in CH₂Cl₂ (20 mL) were added TEA (1.51 g, 14.9 mmol) and DMAP (61 mg, 498 μmol). The mixture was stirred at 0° C. for 5 min, and Ac₂O (508 mg, 5 mmol) was added dropwise at 0° C. The mixture was stirred at 0° C. for 1 hr, Na₂CO₃ (aq. 15 mL) was added to adjust the pH about 8 and extracted with CH₂Cl₂ (60 mL). The organic layer was dried over Na₂SO₄, filtered and concentrated under reduced pressure. After purified by silica gel flash chromatography (Eluent of 0˜5%, EtOAc in petroleum ether), 4-((6-(tert-butyl)-3-nitropyridin-2-yl)amino)benzyl acetate (877 mg, yield: 52%) was obtained as a yellow solid. MS: m/z=344.0 [M+H]⁺. ¹H NMR (400 MHz, Chloroform-d) δ 10.39-10.22 (m, 1H), 8.4 (d, J=8.4 Hz, 1H), 7.77 (d, J=8.0 Hz, 2H), 7.38 (d, J=8.4 Hz, 2H), 6.89 (d, J=8.4 Hz, 1H), 5.11 (s, 2H), 2.12 (s, 3H), 1.36 (s, 9H).

### Step 6: 4-(2-(2-Aminopyridin-3-yl)-5-(tert-butyl)-3H-imidazo[4,5-b]pyridin-3-yl)benzyl acetate

To a solution of 4-((6-(tert-butyl)-3-nitropyridin-2-yl)amino)benzyl acetate in DMSO (30 mL) were added Na₂S₂O₄ (1.85 g, 9 mmol) and 2-aminopyridine-3-carbaldehyde (311 mg, 2.6 mmol). The mixture was stirred at 100° C. for 16 hr. The reaction mixture was poured into H₂O (10 mL), extracted with CH₂Cl₂ (90 mL). The organic phase was dried over anhydrous Na₂SO₄, filtered and concentrated under reduced pressure. After purified by silica gel flash chromatography (Eluent of 0˜72% EtOAc in petroleum ether), 4-(2-(2-aminopyridin-3-yl)-5-(tert-butyl)-3H-imidazo[4,5-b]pyridin-3-yl)benzyl acetate (285 mg, yield: 30%) was obtained as a yellow solid. MS: m/z=416.1 [M+H]⁺.

### Step 7: (4-(2-(2-Aminopyridin-3-yl)-5-(tert-butyl)-3H-imidazo[4,5-b]pyridin-3-yl)phenyl)methanol

To a solution of 4-(2-(2-aminopyridin-3-yl)-5-(tert-butyl)-3H-imidazo[4,5-b]pyridin-3-yl)benzyl acetate (285 mg, 686 μmol) in MeOH (2 mL) and THF (2 mL) were added K₂CO₃ (190 mg, 1.4 mmol) in H₂O (1 mL). The mixture was stirred at 25° C. for 0.5 hr. The reaction mixture was concentrated under reduced pressure to remove MeOH. The residue was diluted with H₂O (5 mL) and extracted with EtOAc (30 mL). The organic layer was dried over Na₂SO₄, filtered and concentrated under reduced pressure to give (4-(2-(2-Aminopyridin-3-yl)-5-(tert-butyl)-3H-imidazo[4,5-b]pyridin-3-yl)phenyl)methanol (216 mg, crude) as a yellow solid. MS: m/z=374.1 [M+H]⁺.

### Step 8: 3-(5-(Tert-butyl)-3-(4-(chloromethyl)phenyl)-3H-imidazo[4,5-b]pyridin-2-yl)pyridin-2-amine

To a solution of (4-(2-(2-aminopyridin-3-yl)-5-(tert-butyl)-3H-imidazo[4,5-b]pyridin-3-yl)phenyl)methanol (216 mg, 578 μmol) in CH₂Cl₂ (5 mL) was added SOCl₂ (206 mg, 1.7 mmol). The mixture was stirred at 40° C. for 0.3 hr. The reaction was concentrated under reduced pressure to give 3-(5-(tert-butyl)-3-(4-(chloromethyl)phenyl)-3H-imidazo[4,5-b]pyridin-2-yl)pyridin-2-amine (Intermediate 123, 200 mg, crude) as a yellow solid. MS: m/z=392.1 [M+H]⁺.

### Intermediate 124: 3-(3-(4-(Chloromethyl)phenyl)-5-ethyl-3H-imidazo[4,5-b]pyridin-2-yl)pyridin-2-amine

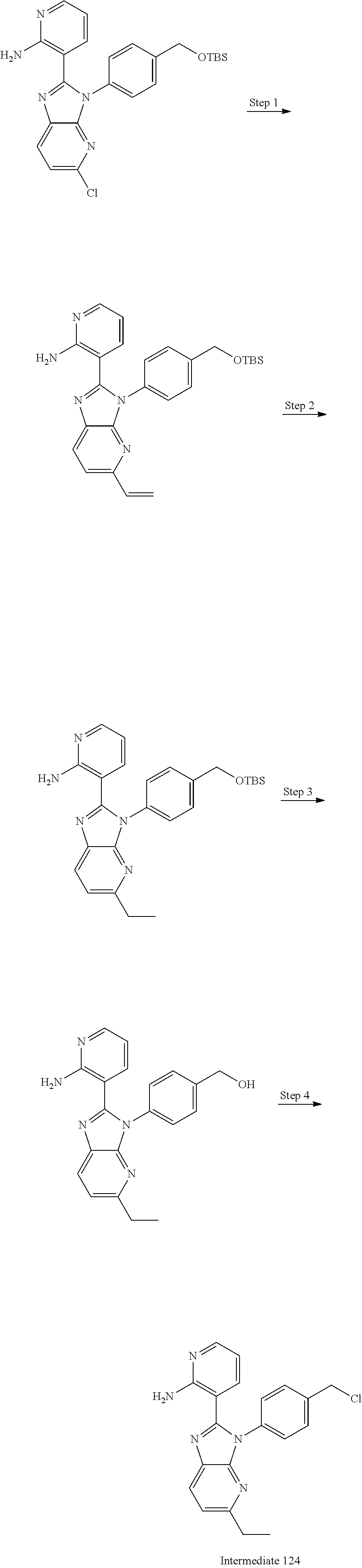 [SMILES: CC(C)(C)[Si](C)(C)OCc1ccc(-n2c(-c3cccnc3N)nc3ccc(Cl)nc32)cc1 ; C=Cc1ccc2nc(-c3cccnc3N)n(-c3ccc(CO[Si](C)(C)C(C)(C)C)cc3)c2n1 ; CCc1ccc2nc(-c3cccnc3N)n(-c3ccc(CO[Si](C)(C)C(C)(C)C)cc3)c2n1 ; CCc1ccc2nc(-c3cccnc3N)n(-c3ccc(CO)cc3)c2n1 ; CCc1ccc2nc(-c3cccnc3N)n(-c3ccc(CCl)cc3)c2n1]

### Step 1: 3-(3-(4-(((Tert-butyldimethylsilyl)oxy)methyl)phenyl)-5-vinyl-3H-imidazo[4,5-b]pyridin-2-yl)pyridin-2-amine

A mixture of 3-(3-(4-(((tert-butyldimethylsilyl)oxy)methyl)phenyl)-5-chloro-3H-imidazo[4,5-b]pyridin-2-yl)pyridin-2-amine (refer to Intermediate 117 for detail procedures, 800 mg, 1.7 mmol), 4,4,5,5-tetramethyl-2-vinyl-1,3,2-dioxaborolane (317 mg, 2 mmol), Cs₂CO₃ (1.7 g, 5 mmol), and Pd(dppf)Cl₂ (126 mg, 172 μmol) in 1,4-dioxane (8 mL) and H₂O (2 mL) was degassed and purged with N₂ three times, then the mixture was stirred at 100° C. for 1.5 hr under N₂ atmosphere. The reaction mixture was diluted with H₂O (30 mL) and extracted with EtOAc (60 mL). The organic layers were washed with brine (30 mL), dried over Na₂SO₄, filtered and concentrated under reduced pressure. After purified by silica gel flash chromatography (Eluent of 0˜35% EtOAc in petroleum ether), 3-(3-(4-(((tert-butyldimethylsilyl)oxy)methyl)phenyl)-5-vinyl-3H-imidazo[4,5-b]pyridin-2-yl)pyridin-2-amine (630 mg, yield: 80%) was obtained as a black brown solid. MS: m/z=458.2 [M+H]⁺. ¹H NMR (400 MHz, Chloroform-d) δ 8.04 (dd, J=4.8, 1.6 Hz, 1H), 8.01 (d, J=8.0 Hz, 1H), 7.47 (d, J=8.4 Hz, 2H), 7.40-7.34 (m, 3H), 7.11 (dd, J=7.6, 1.6 Hz, 1H), 6.85 (dd, J=17.2, 10.8 Hz, 1H), 6.69 (br s, 2H), 6.36 (dd, J=7.6, 5.2 Hz, 1H), 6.15 (dd, 1=17.2, 1.2 Hz, 1H), 5.40 (dd, J=10.8, 1.2 Hz, 1H), 4.84 (s, 2H), 0.97 (s, 9H), 0.14 (s, 6H).

### Step 2: 3-(3-(4-(((Tert-butyldimethylsilyl)oxy)methyl)phenyl)-5-ethyl-3H-imidazo[4,5-b]pyridin-2-yl)pyridin-2-amine

A mixture of 3-(3-(4-(((tert-butyldimethylsilyl)oxy)methyl)phenyl)-5-vinyl-3H-imidazo[4,5-b]pyridin-2-yl)pyridin-2-amine (200 mg, 437 μmol) in MeOH (5 mL) was added Pd/C (80.0 mg, 10% purity), then the mixture was degassed and purged with H₂ three times The mixture was stirred at 25° C. for 4 hr under H₂ atmosphere. The reaction mixture was filtered and the mixture was concentrated under reduced pressure to give 3-(3-(4-(((iert-butyldimethylsilyl)oxy)methyl)phenyl)-5-ethyl-3H-imidazo[4,5-b]pyridin-2-yl)pyridin-2-amine (200 mg, yield. 89%) as yellow oil. MS: m/z=460.2 [M+H]⁺. ¹H NMR (400 MHz, Dimethylsulfoxide-d₆) δ 8.10 (d, J=8.0 Hz, 1H), 7.97 (dd, J=4.4, 1.6 Hz, 1H), 7.48-7.43 (m, 2H), 7.41-7.35 (m, 2H), 7.27 (d, J=8.4 Hz, 1H), 7.13 (dd, J=8.0, 2.0 Hz, 1H), 6.98 (br s, 2H), 6.35 (dd, J=7.6, 4.8 Hz, 1H), 4.80 (s, 2H), 3.19-3.13 (m, 2H), 1.20 (t, J=7.6 Hz, 3H), 0.92 (s, 9H), 0.11 (s, 6H).

### Step 3: (4-(2-(2-Aminopyridin-3-yl)-5-ethyl-3H-imidazo[4,5-b]pyridin-3-yl)phenyl)methanol

To a solution of 3-(3-(4-(((tert-butyldimethylsilyl)oxy)methyl)phenyl)-5-ethyl-3H-imidazo[4,5-b]pyridin-2-yl)pyridin-2-amine (200 mg, 435 μmol) in THF (2 mL) was added TBAF (1 M, 1.74 mL). The mixture was stirred at 25° C. for 0.5 hr. The reaction mixture was diluted with water (10 mL) and extracted with EtOAc (2×10 mL). The combined organic layers were dried over Na₂SO₄, filtered and concentrated under reduced pressure to give (4-(2-(2-aminopyridin-3-yl)-5-ethyl-3H-imidazo[4,5-b]pyridin-3-yl)phenyl)methanol (150 mg, yield: 99¹%) as a yellow solid. MS: m/z=346.1 [M+H]⁺.

### Step 4: 3-(3-(4-(Chloromethyl)phenyl)-5-ethyl-3H-imidazo[4,5-b]pyridin-2-yl)pyridin-2-amine

To a solution of (4-(2-(2-aminopyridin-3-yl)-5-ethyl-3H-imidazo[4,5-b]pyridin-3-yl)phenyl)methanol (150 mg, 434 μmol) in CH₂Cl₂ (2 mL) was added SOCl₂ (206 mg, 1.74 mmol). The mixture was stirred at 40° C. for 0.5 hr. The mixture was concentrated under reduced pressure, 3-(3-(4-(chloromethyl)phenyl)-5-ethyl-3H-imidazo[4,5-b]pyridin-2-yl)pyridin-2-amine (Intermediate 124, 170 mg, HCl salt) was obtained as a yellow solid. MS: m/z=364.1 [M+H]⁺.

### Intermediate 125: 2-(2-Aminopyridin-3-yl)-3-(4-(chloromethyl)phenyl)-N-methyl-3H-imidazo[4,5-b]pyridine-5-carboxamide

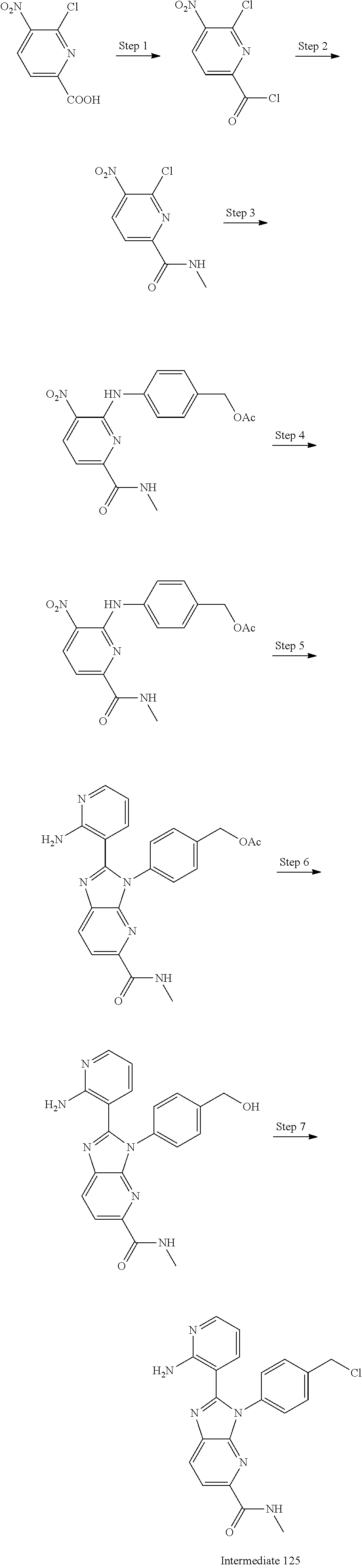

### Step 1: 6-Chloro-5-nitropicolinoyl chloride

A solution of 6-chloro-5-nitropicolinic acid (10 g, 49 mmol) in SOCl₂ (50 mL) was stirred at 80° C. for 16 hr. The reaction mixture was concentrated to give 6-chloro-5-nitropicolinoyl chloride (9.5 g, crude) as a light-green solid.

### Step 2: 6-Chloro-N-methyl-5-nitropicolinamide

To a solution of methylamine (183 mg, 2.71 mmol) in CH₂Cl₂ (5 mL) was added TEA (687 mg, 6.8 mmol) dropwise at 25° C. The mixture was stirred at 25° C. for 5 min, then 6-chloro-5-nitropicolinoyl chloride (500 mg, 2.26 mmol) in CH₂Cl₂ (5 mL) was added dropwise at 0° C. The resulting mixture was stirred at 0° C. for 1 hr. The residue was purified by silica gel flash chromatography (Eluent of 0˜10% EtOAc in petroleum ether) to give 6-chloro-N-methyl-5-nitropicolinamide (308 mg, yield: 63%) as a yellow solid. MS: m/z=215.9 [M+H]⁺. ¹H NMR (400 MHz, Chloroform-d) δ 8.36-8.30 (m, 2H), 7.76 (br s, 1H), 3.07 (d, J=5.2 Hz 3H).

### Step 3: 6-((4-(Hydroxymethyl)phenyl)amino)-N-methyl-5-nitropicolinamide

To a solution of 6-chloro-N-methyl-5-nitropicolinamide (2.3 g, 11 mmol) and (4-aminophenyl)methanol (1.3 g, 11 mmol) in 1,4-dioxane (30 mL) was added DIEA (4.1 g, 32 mmol). The mixture was stirred at 100° C. for 16 hr. The reaction mixture was poured into H₂O (30 mL), extracted with EtOAc (100 mL). The organic phase was dried over anhydrous Na₂SO₄, filtered and concentrated under reduced pressure. The crude 6-((4-(hydroxymethyl)phenyl)amino)-N-methyl-5-nitropicolinamide (3.6 g, crude) was obtained as a red solid. MS: m/z=302.9 [M+H]⁺.

### Step 4: 4-4(6-(Methylcarbamoyl)-3-nitropyridin-2-yl)amino)benzyl acetate

To a solution of 6-((4-(hydroxymethyl)phenyl)amino)-N-methyl-5-nitropicolinamide (1.7 g, 5.6 mmol) in CH₂Cl₂ (10 mL) were added TEA (1.7 g, 17 mmol) and DMAP (69 mg, 562 μmol). The mixture was stirred at 0° C. for 5 min, and then Ac₂O (574 mg, 5.6 mmol) was added dropwise at 0° C. The mixture was stirred at 0° C. for 1 hr. Then sat. Na₂CO₃ (5 mL) was added to adjust pH about 8 and extracted with CH₂Cl₂ (15 mL). The combined layer was dried over Na₂SO₄, filtered and concentrated under reduced pressure. After purified by silica gel flash chromatography (Eluent of 0˜50% EtOAc in petroleum ether), 4-((6-(methylcarbamoyl)-3-nitropyridin-2-yl)amino)benzyl acetate (1.4 g, yield: 72%) was obtained as an orange solid. MS: m/z=345.0 [M+H]⁺. ¹H NMR (400 MHz, Chloroform-d) δ 9.92 (s, 1H), 8.68 (d, J=8.4 Hz, 1H), 7.71 (d, J=8.8 Hz, 1H), 7.54-7.37 (m, 5H), 5.15 (s, 2H), 2.98 (d, J=5.2 Hz, 3H), 2.14 (s, 3H).

### Step 5: 4-(2-(2-Aminopyridin-3-yl)-5-(methylcarbamoyl)-3H-imidazo[4,5-b]pyridin-3-yl)benzyl acetate

To a solution of 4-((6-(methylcarbamoyl)-3-nitropyridin-2-yl)amino)benzyl acetate (1.4 g, 4 mmol) and 2-aminopyridine-3-carbaldehyde (546 mg, 4.5 mmol) in DMSO (15 mL) was added Na₂S₂O₄ (3.3 g, 16 mmol). The mixture was stirred at 100° C. for 16 hr. The reaction mixture was poured into H₂O (30 mL), extracted with CH₂C12 (150 mL). The organic phase was dried over anhydrous Na₂SO₄, filtered and concentrated under reduced pressure. After purified by silica gel flash chromatography (Eluent of 0˜82% EtOAc in petroleum ether), 4-(2-(2-aminopyridin-3-yl)-5-(methylcarbamoyl)-3H-imidazo[4,5-b]pyridin-3-yl)benzyl acetate (650 mg, yield: 38%) was obtained as a yellow oil. MS: m/z=417.1 [M+H]⁺.

### Step 6: 2-(2-Aminopyridin-3-yl)-3-(4-(hydroxymethyl)phenyl)-N-methyl-3H-imidazo[4,5-b]pyridine-5-carboxamide

To a solution of 4-(2-(2-aminopyridin-3-yl)-5-(methylcarbamoyl)-3H-imidazo[4,5-b]pyridin-3-yl)benzyl acetate (420 mg, 1 mmol) in MeOH (8 mL) and THF (8 mL) was added K₂CO₃ (279 mg, 2 mmol) in H₂O (2 mL). The mixture was stirred at 25° C. for 0.5 hr. The reaction mixture was concentrated under reduced pressure to move MeOH. Then the residue was diluted with H₂O (20 mL) and extracted with EtOAc (30 mL). The organic layer was dried over Na₂SO₄, filtered and concentrated under reduced pressure to give 2-(2-aminopyridin-3-yl)-3-(4-(hydroxymethyl)phenyl)-N-methyl-3H-imidazo[4,5-b]pyridine-5-carboxamide (350 mg, crude) as a yellow solid. MS: m/z=375.1 [M+H]⁺.

### Step 7: 2-(2-Aminopyridin-3-yl)-3-(4-(chloromethyl)phenyl)-N-methyl-3H-imidazo[4,5-b]pyridine-5-carboxamide

To a solution of 2-(2-aminopyridin-3-yl)-3-(4-(hydroxymethyl)phenyl)-N-methyl-3H-imidazo[4,5-b]pyridine-5-carboxamide (350 mg, 935 μmol) in CH₂Cl₂ (5 mL) was added SOCl₂ (334 mg, 2.8 mmol). The mixture was stirred at 40° C. for 0.3 hr. The reaction was concentrated under reduced pressure to give 2-(2-aminopyridin-3-yl)-3-(4-(chloromethyl)phenyl)-N-methyl-3H-imidazo[4,5-b]pyridine-5-carboxamide (Intermediate 125, 200 mg, HCl salt, crude) as a yellow solid. MS: m/z=393.0 [M+H]⁺.

### Intermediate 126: 4-(2-(2-Aminopyridin-3-yl)-3-(4-(chloromethyl)phenyl)-3H-imidazo[4,5-b]pyridin-5-yl)nicotinonitrile

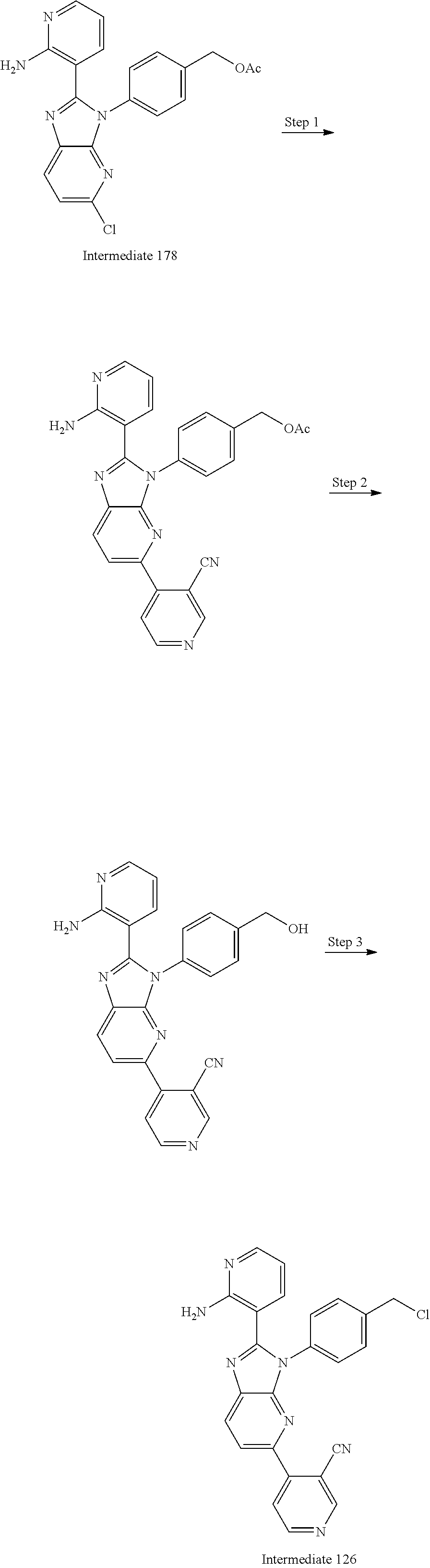 [SMILES: CC(=O)OCc1ccc(-n2c(-c3cccnc3N)nc3ccc(Cl)nc32)cc1 ; CC(=O)OCc1ccc(-n2c(-c3cccnc3N)nc3ccc(-c4ccncc4C#N)nc32)cc1 ; N#Cc1cnccc1-c1ccc2nc(-c3cccnc3N)n(-c3ccc(CO)cc3)c2n1 ; N#Cc1cnccc1-c1ccc2nc(-c3cccnc3N)n(-c3ccc(CCl)cc3)c2n1]

### Step 1: 4-(2-(2-Aminopyridin-3-yl)-5-(3-cyanopyridin-4-yl)-3H-imidazo[4,5-b]pyridin-3-yl)benzyl acetate

A mixture of Intermediate 178 (400 mg, 1.02 mmol), 4-(4,4,5,5-tetramethyl-1,3,2-dioxaborolan-2-yl)nicotinonitrile (257 mg, 1.12 mmol), cataCXiumAPdG3 (369 mg, 507 μmol), K₃PO₄ (431 mg, 2.03 mmol) and PCy₃ (28.4 mg, 101 μmol) in DMF (10 mL) was degassed and purged with N₂ three times, and then the mixture was stirred at 120° C. for 12 hr under N₂ atmosphere. The reaction mixture was diluted with water (20 mL) and extracted with EtOAc (2×30 mL). The combined organic layers were washed with brine (10 mL), dried over Na₂SO₄, filtered and concentrated under reduced pressure. After purified by silica gel flash chromatography (Eluent of 0˜8% MeOH in CH₂Cl₂), 4-(2-(2-aminopyridin-3-yl)-5-(3-cyanopyridin-4-yl)-3H-imidazo[4,5-b]pyridin-3-yl)benzyl acetate (450 mg, yield: 76%) was obtained as a red solid. MS: m/z=462.1 [M+H]⁺.

### Step 2: 4-(2-(2-Aminopyridin-3-yl)-3-(4-(hydroxymethyl)phenyl)-3H-imidazo[4,5-b]pyridin-5-yl)nicotinonitrile

To a solution of 4-(2-(2-aminopyridin-3-yl)-5-(3-cyanopyridin-4-yl)-3H-imidazo[4,5-b]pyridin-3-yl)benzyl acetate (450 mg, 975 μmol) in MeOH (3 mL) and THF (3 mL) was added a solution of K₂CO₃ (134 mg, 975 μmol) in H₂O (1 mL). The mixture was stirred at 25° C. for 0.5 hr. The reaction mixture was diluted with water (10 mL) and extracted with EtOAc (2×10 mL). The combined organic layers were washed with brine (10 mL), dried over Na₂SO₄, filtered, and concentrated under reduced pressure to give 4-(2-(2-aminopyridin-3-yl)-3-(4-(hydroxymethyl)phenyl)-3H-imidazo[4,5-b]pyridin-5-yl)nicotinonitrile (300 mg, yield: 73%) as a yellow solid. MS: m/z=420.1 [M+H]⁺.

### Step 3: 4-(2-(2-Aminopyridin-3-yl)-3-(4-(chloromethyl)phenyl)-3H-imidazo[4,5-b]pyridin-5-yl)nicotinonitrile

To a solution of 4-(2-(2-aminopyridin-3-yl)-3-(4-(hydroxymethyl)phenyl)-3H-imidazo[4,5-b]pyridin-5-yl)nicotinonitrile (300 mg, 715 μmol) in CH₂Cl₂ (5 mL) was added SOCl₂ (340 mg, 2.86 mmol). The mixture was stirred at 40° C. for 0.5 hr. The mixture was concentrated under reduced pressure to give 4-(2-(2-aminopyridin-3-yl)-3-(4-(chloromethyl)phenyl)-3H-imidazo[4,5-b]pyridin-5-yl)nicotinonitrile (Intermediate 126, 300 mg, HCl salt) as a yellow solid. MS: m/z=437.9 [M+H]⁺.

### Intermediate 127: 3-(3-(4-(Chloromethyl)phenyl)-5-fluoro-3H-imidazo[4,5-b]pyridin-2-yl)pyridin-2-amine

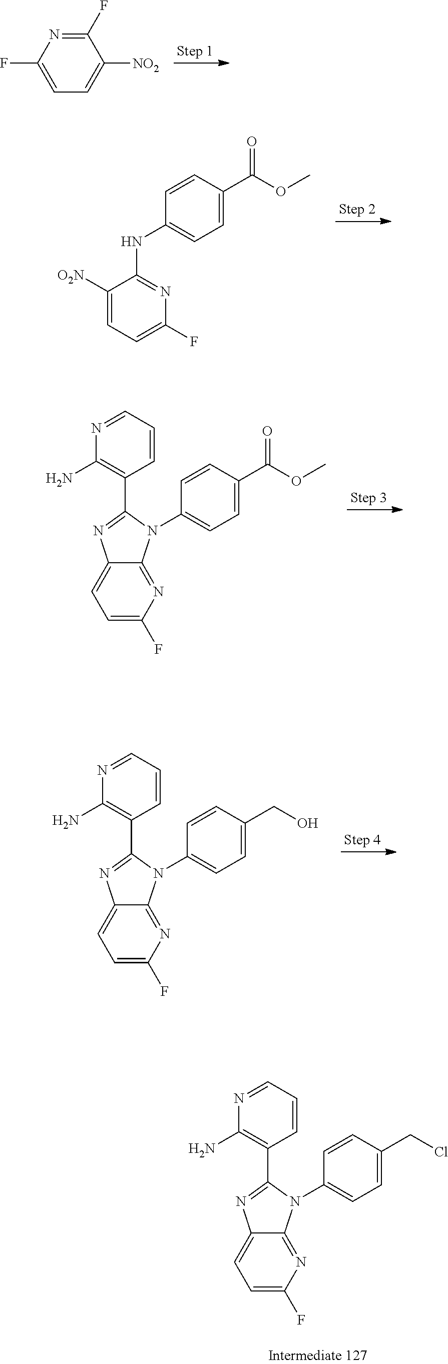 [SMILES: O=[N+]([O-])c1ccc(F)nc1F ; COC(=O)c1ccc(Nc2nc(F)ccc2[N+](=O)[O-])cc1 ; COC(=O)c1ccc(-n2c(-c3cccnc3N)nc3ccc(F)nc32)cc1 ; Nc1ncccc1-c1nc2ccc(F)nc2n1-c1ccc(CO)cc1 ; Nc1ncccc1-c1nc2ccc(F)nc2n1-c1ccc(CCl)cc1]

### Step 1: Methyl 4-((6-fluoro-3-nitropyridin-2-yl)amino)benzoate

To a solution of methyl 4-aminobenzoate (10 g, 66.1 mmol) and 2,6-difluoro-3-nitropyridine (10.5 g, 66 mmol) in dioxane (175 mL) was added DIEA (17.1 g, 132 mmol). The mixture was stirred at 80° C. for 12 hr. The reaction mixture was diluted with water (100 mL) and extracted with EtOAc (2×200 mL). The combined organic layers were dried over Na₂SO₄, filtered and concentrated under reduced pressure. After triturated with EtOAc (100 mL) and filtered, methyl 4-((6-fluoro-3-nitropyridin-2-yl)amino)benzoate (13.4 g, yield: 60%) was obtained as a yellow solid. MS: m/z=292.0 [M+H]⁺. ¹H NMR (400 MHz, Chloroform-d) δ 10.53 (br s, 1H), 8.74-8.62 (m, 1H), 8.07 (d, J=8.4 Hz, 2H), 7.76 (d, J=8.8 Hz, 2H), 6.49 (dd, J=8.8, 3.6 Hz, 1H), 3.92 (s, 3H), ¹⁹F NMR (400 MHz, Chloroform-d) δ −52.533.

### Step 2: Methyl 4-(2-(2-aminopyridin-3-yl)-5-fluoro-3H-imidazo[4,5-b]pyridin-3-yl)benzoate

To a solution of methyl 4-((6-fluoro-3-nitropyridin-2-yl)amino)benzoate (6.2 g, 21.2 mmol) and 2-aminonicotinaldehyde (2.86 g, 23.4 mmol) in DMSO (186 mL) was added Na₂S₂O₄ (17 g, 85.1 mmol, 87% purity). The mixture was stirred at 100° C. for 12 hr. The reaction mixture was diluted with water (50 mL) and extracted with CH₂Cl₂ (2×250 mL). The combined organic layers were washed with brine (100 mL), dried over Na₂SO₄, filtered and concentrated under reduced pressure. After purified by silica gel flash chromatography (Eluent of 0˜100% EtOAc in petroleum ether), methyl 4-(2-(2-aminopyridin-3-yl)-5-fluoro-3H-imidazo[4,5-b]pyridin-3-yl)benzoate (1.5 g, yield: 18%) was obtained as a yellow solid. MS: m/z=364.1 [M+H]+. ¹H NMR (400 MHz, Chloroform-d) δ 8.22-8.14 (m, 3H), 8.08 (dd, J=4.8, 1.6 Hz, 1H), 7.48-7.42 (m, 2H), 7.01 (dd, J=8.0, 1.6 Hz, 1H), 6.96 (d, J=8.4 Hz, 1H), 6.56 (br s, 2H), 6.37 (dd, J=8.0, 4.8 Hz, 1H), 3.97 (s, 3H), ¹⁹F NMR (400 MHz, Chloroform-d) δ −71.619.

### Step 3: (4-(2-(2-Aminopyridin-3-yl)-5-fluoro-3H-imidazo[4,5-b]pyridin-3-yl)phenyl)methanol

To a solution of methyl 4-(2-(2-aminopyridin-3-yl)-5-fluoro-3H-imidazo[4,5-b]pyridin-3-yl)benzoate (1.5 g, 4.13 mmol) in THF (30 mL) was added LiAlH₄ (2.5 M, 2.48 mL). The mixture was stirred at 0° C. for 2 hr. The reaction mixture was quenched with Na₂SO₄·10H₂O (282 mg) at 0° C. and filtered. The filtrate was concentrated under reduced pressure to give (4-(2-(2-aminopyridin-3-yl)-5-fluoro-3H-imidazo[4,5-b]pyridin-3-yl)phenyl)methanol (1.3 g) as a yellow solid.

### Step 4: 3-(3-(4-(Chloromethyl)phenyl)-5-fluoro-3H-imidazo[4,5-b]pyridin-2-yl)pyridin-2-amine

To a solution of (4-(2-(2-aminopyridin-3-yl)-5-fluoro-3H-imidazo[4,5-b]pyridin-3-yl)phenyl)methanol (1.3 g, 3.88 mmol) in CH₂Cl₂ (10 mL) was added SOCl₂ (2.77 g, 23.3 mmol). The mixture was stirred at 40° C. for 0.5 hr. The mixture was concentrated under reduced pressure. The residue was purified by silica gel flash chromatography (Eluent of 0˜6% MeOH in CH₂Cl₂), 3-(3-(4-(chloromethyl)phenyl)-5-fluoro-3H-imidazo[4,5-b]pyridin-2-yl)pyridin-2-amine (Intermediate 127, 400 mg, yield: 27%) was obtained as a yellow solid. MS: m/z=353.9 [M+H]+.

### Intermediate 128: 3-(3-(4-(Chloromethyl)phenyl)-6-fluoro-3H-imidazo[4,5-b]pyridin-2-yl)pyridin-2-amine

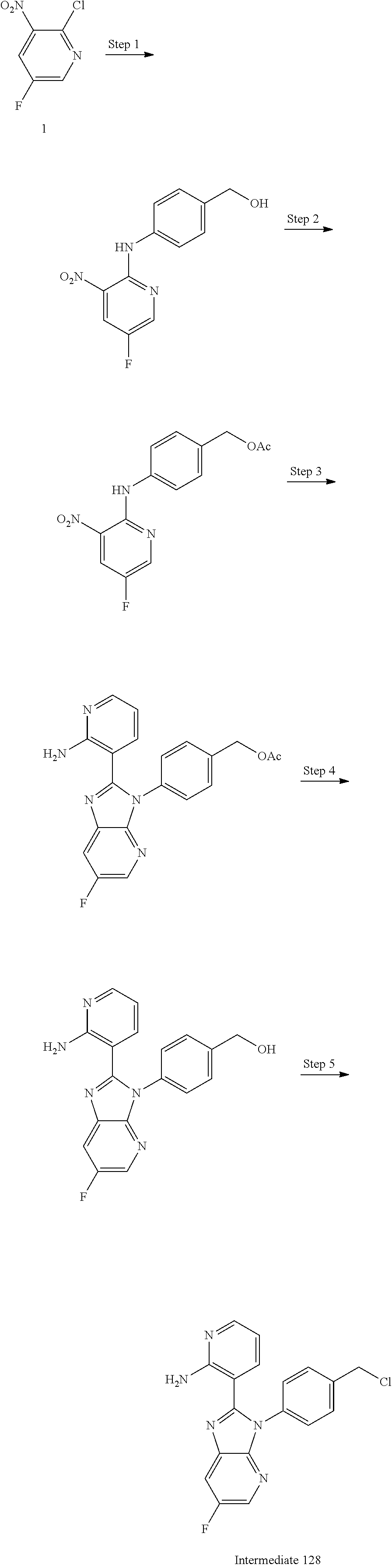 [SMILES: O=[N+]([O-])c1cc(F)cnc1Cl ; O=[N+]([O-])c1cc(F)cnc1Nc1ccc(CO)cc1 ; CC(=O)OCc1ccc(Nc2ncc(F)cc2[N+](=O)[O-])cc1 ; CC(=O)OCc1ccc(-n2c(-c3cccnc3N)nc3cc(F)cnc32)cc1 ; Nc1ncccc1-c1nc2cc(F)cnc2n1-c1ccc(CO)cc1 ; Nc1ncccc1-c1nc2cc(F)cnc2n1-c1ccc(CCl)cc1]

### Step 1: (4-((5-Fluoro-3-nitropyridin-2-yl)amino)phenyl)methanol

To a solution of 2-chloro-5-fluoro-3-nitro-pyridine (4 g, 23 mmol) in 1,4-dioxane (40 mL) were added DIEA (7.3 g, 57 mmol) and (4-aminophenyl)methanol (2.8 g, 23 mmol). The mixture was stirred at 110° C. for 16 hr. The reaction mixture was diluted with H₂O (200 mL) and extracted with EtOAc (400 mL). The organic layer was washed with brine (130 mL), dried over Na₂SO₄, filtered and concentrated under reduced pressure to give (4-((5-fluoro-3-nitropyridin-2-yl)amino)phenyl)methanol (3.8 g, crude) as a yellow solid. MS: m/z=263.9 [M+H]⁺.

### Step 2: 4-((5-Fluoro-3-nitropyridin-2-yl)amino)benzyl acetate

To a solution of (4-((5-fluoro-3-nitropyridin-2-yl)amino)phenyl)methanol (3.8 g, 14 mmol) and Ac₂O (1.5 g, 14 mmol) in CH₂Cl₂ (38 mL) was added TEA (4.4 g, 43 mmol) and DMAP (176 mg, 1.4 mmol) at 0° C. The mixture was stirred at 0° C. for 1 hr. The reaction mixture was diluted with H₂O (150 mL) and extracted with CH₂Cl₂ (400 mL). The organic layer was washed with brine (150 mL), dried over Na₂SO₄, filtered and concentrated under reduced pressure. After purified by silica gel flash chromatography (Eluent of 10˜30%, EtOAc in petroleum ether), 4-((5-fluoro-3-nitropyridin-2-yl)amino)benzyl acetate (1.4 g, yield: 30%) was obtained as a yellow solid. MS: m/z=305.9 [M+H]⁺. ¹H NMR (400 MHz, Chloroform-d) δ 10.02 (br s, 1H), 8.44 (d, J=2.4 Hz, 1H), 8.30 (dd, J=7.6, 2.8 Hz, 1H), 7.61 (d, J=8.4 Hz, 2H), 7.39 (d, J=8.0 Hz, 2H), 5.11 (s, 2H), 2.11 (s, 3H).

### Step 3: 4-(2-(2-Aminopyridin-3-yl)-6-fluoro-3H-imidazo[4,5-b]pyridin-3-yl)benzyl acetate

A mixture of 4-((5-fluoro-3-nitropyridin-2-yl)amino)benzyl acetate (1.4 g, 4.6 mmol), 2-aminopyridine-3-carbaldehyde (616 mg, 5 mmol), Na₂S₂O₄ (3.2 g, 18 mmol) in DMSO (40 mL) was degassed and purged with N₂ three times, and then the mixture was stirred at 100° C. for 16 hr under N₂ atmosphere. The reaction mixture was diluted with H₂O (200 mL) and extracted with CH₂Cl₂ (200 mL×3). The combined organic layers were washed with brine (150 mL), dried over Na₂SO₄, filtered and concentrated under reduced pressure. After purified by silica gel flash chromatography (Eluent of 10˜20% EtOAc in petroleum ether), 4-(2-(2-aminopyridin-3-yl)-6-fluoro-3H-imidazo[4,5-b]pyridin-3-yl)benzyl acetate (285 mg, yield: 11%) was obtained as a brown solid. MS: m/z=378.0 [M+H]⁺. ¹H NMR (400 MHz, Chloroform-d) δ 8.35-8.23 (m, 1H), 8.06 (dd, J=5.2, 1.6 Hz, 1H), 7.82 (dd, J=5.2, 1.6 Hz, 1H), 7.54 (d, J=8.4 Hz, 2H), 7.38 (d, J=8.4 Hz, 2H), 7.13 (dd, J=8.0, 1.6 Hz, 1H), 6.89 (br s, 2H), 6.41 (dd, J=8.0, 5.2 Hz, 1H), 5.20 (s, 2H), 2.15 (s, 3H).

### Step 4: (4-(2-(2-Aminopyridin-3-yl)-6-fluoro-3H-imidazo[4,5-b]pyridin-3-yl)phenyl)methanol

To a solution of 4-(2-(2-aminopyridin-3-yl)-6-fluoro-3H-imidazo[4,5-b]pyridin-3-yl)benzyl acetate (285 mg, 755 μmol) in THF (2 mL) and MeOH (2 mL) was added K₂CO₃ (313 mg, 2.3 mmol) in H₂O (1 mL). The mixture was stirred at 25° C. for 0.5 hr. The mixture was concentrated under reduced pressure. The residue was diluted with H₂O (10 mL) and extracted with CH₂Cl₂ (20 mL). The organic layer was washed with brine (10 mL), dried over Na₂SO₄, filtered and concentrated under reduced pressure. (4-(2-(2-Aminopyridin-3-yl)-6-fluoro-3H-imidazo[4,5-b]pyridin-3-yl)phenyl)methanol (230 mg, crude) was obtained as a brown solid. MS: m/z=336.0 [M+H]⁺.

### Step 5: 3-(3-(4-(Chloromethyl)phenyl)-6-fluoro-3H-imidazo[4,5-b]pyridin-2-yl)pyridin-2-amine

To a solution of (4-(2-(2-aminopyridin-3-yl)-6-fluoro-3H-imidazo[4,5-b]pyridin-3-yl)phenyl)methanol (230 mg, 686 μmol) in CH₂Cl₂ (5 mL) was added SOCl₂ (163 mg, 1 mmol). The mixture was stirred at 40° C. for 0.5 hr. The mixture was concentrated under reduced pressure. 3-(3-(4-(Chloromethyl)phenyl)-6-fluoro-3H-imidazo[4,5-b]pyridin-2-yl)pyridin-2-amine (Intermediate 128, 284 mg, crude) was obtained as a yellow solid. MS: m/z=353.9 [M+H]⁺.

### Intermediate 129: 3-(5-Bromo-3-(4-(((tert-butyldimethylsilyl)oxy)methyl)phenyl)-3H-imidazo[4,5-b]pyridin-2-yl)pyridin-2-amine

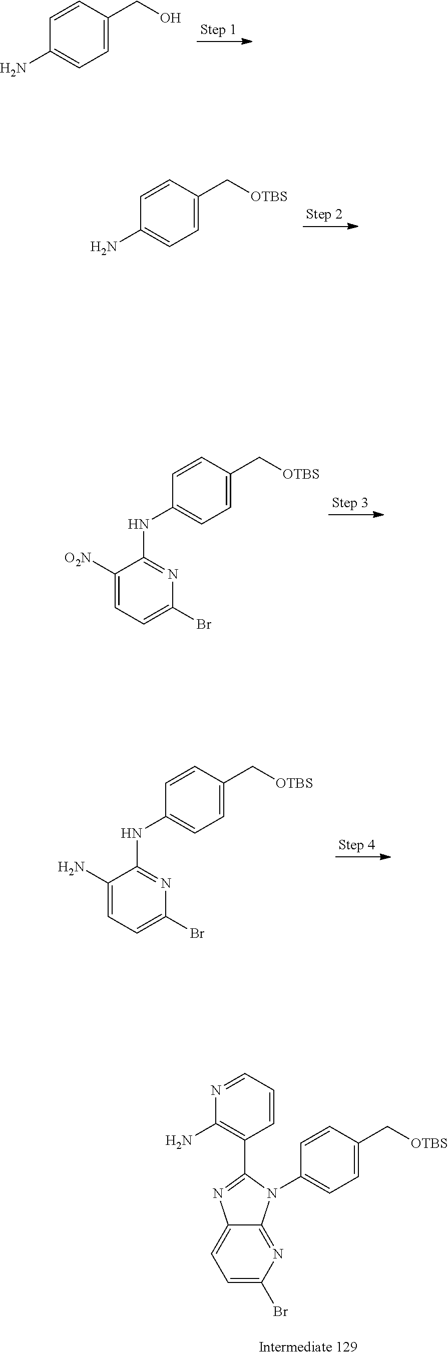 [SMILES: Nc1ccc(CO)cc1 ; CC(C)(C)[Si](C)(C)OCc1ccc(N)cc1 ; CC(C)(C)[Si](C)(C)OCc1ccc(Nc2nc(Br)ccc2[N+](=O)[O-])cc1 ; CC(C)(C)[Si](C)(C)OCc1ccc(Nc2nc(Br)ccc2N)cc1 ; CC(C)(C)[Si](C)(C)OCc1ccc(-n2c(-c3cccnc3N)nc3ccc(Br)nc32)cc1]

### Step 1: 4-(((tert-Butyldimethylsilyl)oxy)methyl)aniline

To a solution of (4-aminophenyl)methanol (5 g, 40.6 mmol) in CH₂Cl₂ (50 mL) were added TBSCl (8.34 g, 55.4 mmol), TEA (10.3 mL, 73.8 mmol), and DMAP (225 mg, 1.85 mmol). The mixture was degassed and purged with N₂ three times, and stirred at 25° C. for 2 hr under N₂. The reaction mixture was filtered and concentrated under reduced pressure to give 4-(((tert-butyldimethylsilyl)oxy)methyl)aniline (8.76 g, crude) as a white oil, which was used in the next step without further purification.

### Step 2: 6-Bromo-N-(4-(((tert-butyldimethylsilyl)oxy)methyl)phenyl)-3-nitropyridin-2-amine

To a solution of 4-[[tert-butyl(dimethyl)silyl]oxymethyl]aniline (8.76 g, 36.9 mmol) in 1,4-dioxane (100 mL) were added DIEA (19.3 mL, 111 mmol) and 2,6-dibromo-3-nitropyridine (10.4 g, 36.9 mmol). The mixture was degassed and purged with N₂ three times and stirred at 45° C. for 16 hr under N₂. The reaction mixture was concentrated under reduced pressure and purified by silica gel flash chromatography (Eluent of 1˜10% EtOAc in Petroleum ether) to give 6-bromo-N-(4-(((tert-butyldimethylsilyl)oxy)methyl)phenyl)-3-nitropyridin-2-amine (13.7 g, yield: 72%) as a red solid. MS: m/z=440.2 [M+H]⁺. ¹H NMR (400 MHz, Chloroform-d) δ 10.23 (s, 1H), 8.32 (d, J=8.4 Hz, 1H), 7.61 (d, J=8.4 Hz, 2H), 6.95 (d, J=8.4 Hz, 1H), 4.75 (s, 2H), 0.96 (s, 9H), 0.12 (s, 6H).

### Step 3: 6-Bromo-N²-(4-(((tert-butyldimethylsilyl)oxy)methyl)phenyl)pyridine-2,3-diamine

To a solution of 6-bromo-N-(4-(((tert-butyldimethylsilyl)oxy)methyl)phenyl)-3-nitropyridin-2-amine (46 g, 105 mmol) in EtOH (500 mL) and H₂O (100 mL) were added Fe (17.6 g, 315 mmol) and NH₄Cl (39.3 g, 735 mmol). The mixture was degassed and purged with N₂ three times and stirred at 80° C. for 2 hr under N₂. The reaction mixture was filtered and concentrated under reduced pressure. 6-Bromo-N²-(4-(((tert-butyldimethylsilyl)oxy)methyl)phenyl)pyridine-2,3-diamine (42.9 g, crude) was obtained as a yellow solid, which was used in the next step without further purification. MS: m/z=408.9, 409.9 [M+H]⁺.

### Step 4: 3-(5-Bromo-3-(4-(((tert-butyldimethylsilyl)oxy)methyl)phenyl)-3H-imidazo[4,5-b]pyridin-2-yl)pyridin-2-amine

To a solution of 6-bromo-N²-(4-(((tert-butyldimethylsilyl)oxy)methyl)phenyl)pyridine-2,3-diamine (41 g, 100 mmol), 2-aminonicotinaldehyde (9.81 g, 80.3 mmol) in EtOH (500 mL) was added InCh (2.22 g, 10.0 mmol). The mixture was degassed and purged with N₂ three times and stirred at 80° C. for 2 hr under N₂. Then MnO₂ (52.4 g, 602 mmol) was added. The mixture was stirred at 80° C. for 2 hr under N₂. The reaction mixture was filtered and concentrated under reduced pressure. The crude was purified by silica gel flash chromatography (Eluent of 1˜50% EtOAc in petroleum ether) to give 3-(5-bromo-3-(4-(((tert-butyldimethylsilyl)oxy)methyl)phenyl)-3H-imidazo[4,5-b]pyridin-2-yl)pyridin-2-amine (Intermediate 129, 19.5 g, yield: 32%) as a brown solid. MS: m/z=509.9, 511.9 [M+H]⁺. ¹H NMR (400 MHz, Chloroform-d) δ 8.10-8.01 (m, 1H), 7.92 (d, 1=8.4 Hz, 1H), 7.54-7.40 (m, 3H), 7.31 (d, J=8.0 Hz, 2H), 7.07 (d, J=7.6 Hz, 1H), 6.64 (br s, 2H), 6.41-6.27 (m, 1H), 4.83 (s, 2H), 0.97 (s, 9H), 0.14 (s, 6H).

### Intermediate 130: 3-(2-(2-Aminopyridin-3-yl)-3-(4-(chloromethyl)phenyl)-3H-imidazo[4,5-b]pyridin-5-yl)oxazolidin-2-one

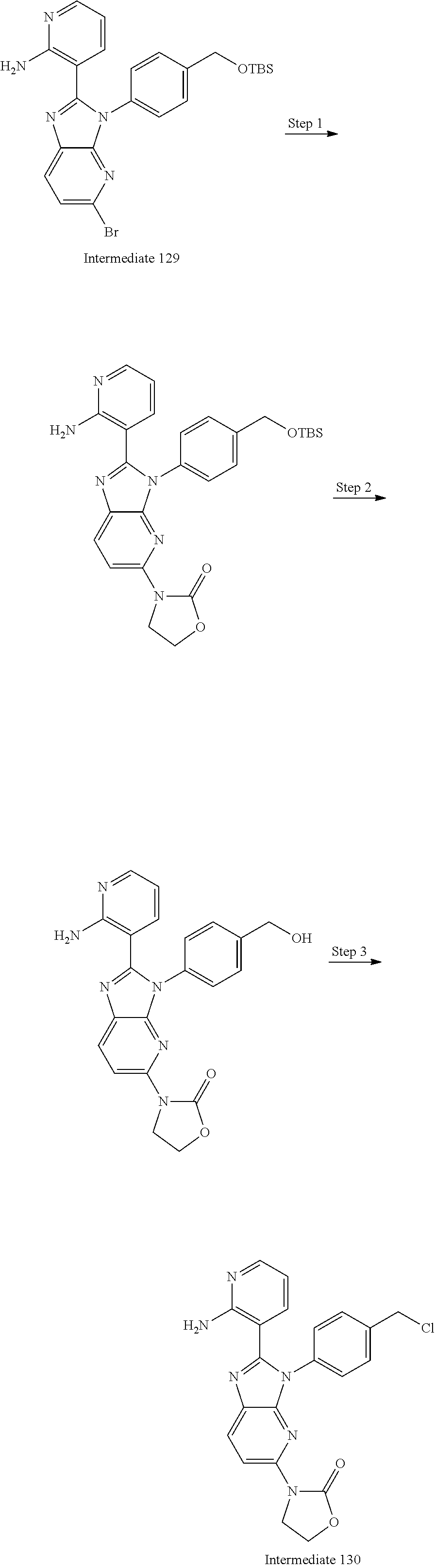 [SMILES: CC(C)(C)[Si](C)(C)OCc1ccc(-n2c(-c3cccnc3N)nc3ccc(Br)nc32)cc1 ; CC(C)(C)[Si](C)(C)OCc1ccc(-n2c(-c3cccnc3N)nc3ccc(N4CCOC4=O)nc32)cc1 ; Nc1ncccc1-c1nc2ccc(N3CCOC3=O)nc2n1-c1ccc(CO)cc1 ; Nc1ncccc1-c1nc2ccc(N3CCOC3=O)nc2n1-c1ccc(CCl)cc1]

### Step 1: 3-(2-(2-Aminopyridin-3-yl)-3-(4-(((tert-butyldimethylsilyl)oxy)methyl)phenyl)-3H-imidazo[4,5-b]pyridin-5-yl)oxazolidin-2-one

A mixture of Intermediate 129 (800 mg, 783 μmol), oxazolidin-2-one (82 mg, 940 μmol), CuI (8 mg, 39 μmol), K₂CO₃ (216 mg, 1.57 mmol), and dimethylaminoactic acid (8 mg, 78.3 μmol) in DMF (8 mL) was degassed and purged with N₂ three times, and then the mixture was stirred at 120° C. for 16 hr under N₂ atmosphere. The reaction mixture was diluted with H₂O (100 mL) and extracted with EtOAc (30 mL×2). The combined organic layers were washed with brine (50 mL), dried over Na₂SO₄, filtered and concentrated under reduced pressure. The residue was purified by silica gel flash chromatography (Eluent of 0˜7% MeOH in CH₂Cl₂) to give 3-(2-(2-aminopyridin-3-yl)-3-(4-(((tert-butyldimethylsilyl)oxy)methyl)phenyl)-3H-imidazo[4,5-b]pyridin-5-yl)oxazolidin-2-one (300 mg, yield: 33%) as a brown solid. MS: m/z=517.1 [M+H]⁺.

### Step 2: 3-(2-(2-Aminopyridin-3-yl)-3-(4-(hydroxymethyl)phenyl)-3H-imidazo[4,5-b]pyridin-5-yl)oxazolidin-2-one

A solution of 3-(2-(2-aminopyridin-3-yl)-3-(4-(((tert-butyldimethylsilyl)oxy)methyl)phenyl)-3H-imidazo[4,5-b]pyridin-5-yl)oxazolidin-2-one (300 mg, 580 μmol) in TBAF (1 M, 4.69 mL) was stirred at 25° C. for 1 hr. The reaction mixture was diluted with H₂O (30 mL) and extracted with CH₂Cl₂ (30 mL×2). The combined organic layers were washed with brine (30 mL), dried over Na₂SO₄, filtered and concentrated under reduced pressure. The residue was purified by silica gel flash chromatography (Eluent of 0˜7% MeOH in CH₂Cl₂) to give 3-(2-(2-aminopyridin-3-yl)-3-(4-(hydroxymethyl)phenyl)-3H-imidazo[4,5-b]pyridin-5-yl)oxazolidin-2-one (90 mg, yield: 38%) as a yellow solid. MS: m/z=403.1 [M+H]⁺. ¹H NMR (400 MHz, Chloroform-d) δ 8.27 (d, J=8.8 Hz, 1H), 8.10 (d, J=8.8 Hz, 1H), 8.05 (dd, J=5.2, 1.6 Hz, 1H), 7.50 (d, J=8.4 Hz, 2H), 7.35 (d, J=8.4 Hz, 2H), 7.07 (dd, J=7.6, 1.6 Hz, 1H), 6.57 (br s, 2H), 6.37 (dd, J=7.6, 5.2 Hz, 1H), 4.82 (s, 2H), 4.48-4.40 (m, 2H), 4.25-4.16 (m, 2H).

### Step 3: 3-(2-(2-Aminopyridin-3-yl)-3-(4-(chloromethyl)phenyl)-3H-imidazo[4,5-b]pyridin-5-yl)oxazolidin-2-one

To a solution of 3-(2-(2-aminopyridin-3-yl)-3-(4-(hydroxymethyl)phenyl)-3H-imidazo[4,5-b]pyridin-5-yl)oxazolidin-2-one (80 mg, 198 μmol) in CH₂Cl₂ (2 mL) was added SOCl₂ (142 mg, 1.19 mmol). The mixture was stirred at 40° C. for 1 hr. The reaction mixture was concentrated under reduced pressure. The crude 3-(2-(2-aminopyridin-3-yl)-3-(4-(chloromethyl)phenyl)-3H-imidazo[4,5-b]pyridin-5-yl)oxazolidin-2-one (Intermediate 130, 90 mg, HCl salt, yield: 100%) was used in the next step without further purification. MS: m/z=420.9 [M+H]⁺.

### Intermediate 131: 3-(3-(4-(Chloromethyl)phenyl)-5-(6-fluoropyridin-3-yl)-3H-imidazo[4,5-b]pyridin-2-yl)pyridin-2-amine

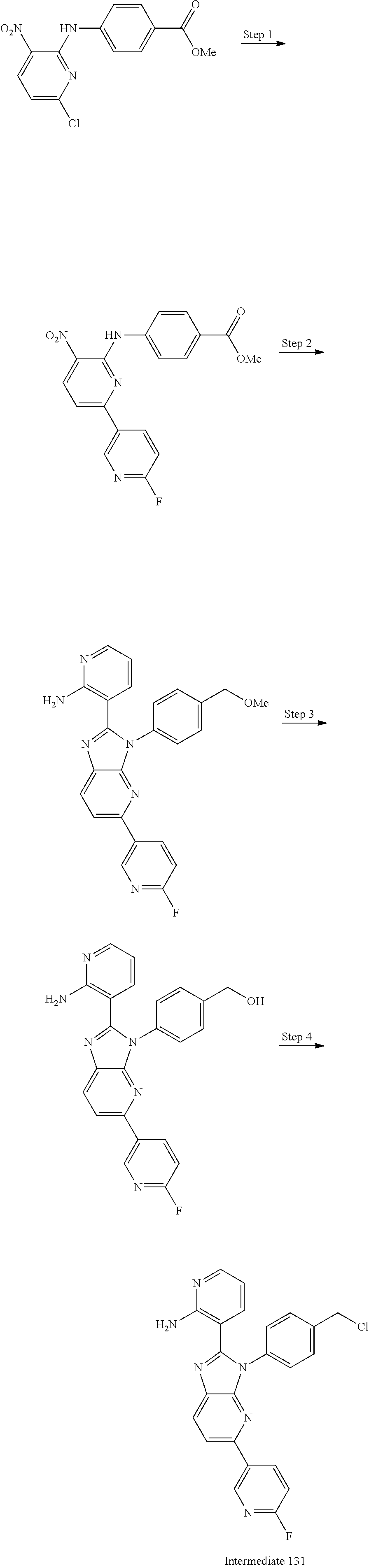 [SMILES: COC(=O)c1ccc(Nc2nc(Cl)ccc2[N+](=O)[O-])cc1 ; COC(=O)c1ccc(Nc2nc(-c3ccc(F)nc3)ccc2[N+](=O)[O-])cc1 ; COCc1ccc(-n2c(-c3cccnc3N)nc3ccc(-c4ccc(F)nc4)nc32)cc1 ; Nc1ncccc1-c1nc2ccc(-c3ccc(F)nc3)nc2n1-c1ccc(CO)cc1 ; Nc1ncccc1-c1nc2ccc(-c3ccc(F)nc3)nc2n1-c1ccc(CCl)cc1]

### Step 1: Methyl 4-((6′-fluoro-5-nitro-[2,3′-bipyridin]-6-yl)amino)benzoate

A mixture of methyl 4-((6-chloro-3-nitropyridin-2-yl)amino)benzoate (refer to Intermediate 13 for detail procedures, 10 g, 33 mmol), (6-fluoro-3-pyridyl)boronic acid (5 g, 36 mmol), Pd(dppf)Cl₂ (2.4 g, 3.3 mmol), and Cs₂CO₃ (31.8 g, 97.5 mmol) in 1,4-dioxane (100 mL) and H₂O (10 mL) was degassed and purged with N₂ three times, then the mixture was stirred at 100° C. for 16 hr under N₂ atmosphere. The reaction mixture was diluted with water (200 mL) and extracted with EtOAc (3×300 mL). The combined organic layers were washed with brine (300 mL), dried over Na₂SO₄, filtered and concentrated under reduced pressure. After purified by silica gel flash chromatography (Eluent of 0˜100% EtOAc in petroleum ether), methyl 4-((6′-fluoro-5-nitro-[2,3′-bipyridin]-6-yl)amino)benzoate (6.6 g, yield: 33%) was obtained as a yellow solid. MS: m/z=369.0 [M+H]⁺. ¹H NMR (400 MHz, Chloroform-d) δ 10.41 (s, 1H), 8.90 (d, J=2.4 Hz, 1H), 8.67 (d, J=8.4 Hz, 1H), 8.48-8.43 (m, 1H), 8.12 (d, J=8.8 Hz, 2H), 7.80 (d, J=8.8 Hz, 2H), 7.36 (d, J=8.8 Hz, 1H), 7.09 (dd, J=8.4, 2.8 Hz, 1H), 3.94 (s, 3H)

### Step 2. Methyl 4-(2-(2-aminopyridin-3-yl)-5-(6-fluoropyridin-3-yl)-3H-imidazo[4,5-b]pyridin-3-yl)benzoate

A mixture of methyl 4-((6′-fluoro-5-nitro-[2,3′-bipyridin]-6-yl)amino)benzoate (6.6 g, 18 mmol), 2-aminopyridine-3-carbaldehyde (2.41 g, 19.7 mmol), and Na₂S₂O₄ (12.5 g, 71.7 mmol) in DMSO (180 mL) was degassed and purged with N₂ three times. The mixture was stirred at 100° C. for 16 hr under N₂ atmosphere. The reaction mixture was diluted with water (200 mL) and extracted with EtOAc (3×300 mL). The combined organic layers were washed with brine (300 mL), dried over Na₂SO₄, filtered and concentrated under reduced pressure. After purified by silica gel flash chromatography (Eluent of 0˜100%, EtOAc in petroleum ether), methyl 4-(2-(2-aminopyridin-3-yl)-5-(6-fluoropyridin-3-yl)-3H-imidazo[4,5-b]pyridin-3-yl)benzoate (5 g, yield: 62%) was obtained as a yellow solid. MS: m/z=441.0 [M+H]⁺.

### Step 3: (4-(2-(2-Aminopyridin-3-yl)-5-(6-fluoropyridin-3-yl)-3H-imidazo[4,5-b]pyridin-3-yl)phenyl)methanol

To a mixture of methyl 4-(2-(2-aminopyridin-3-yl)-5-(6-fluoropyridin-3-yl)-3H-imidazo[4,5-b]pyridin-3-yl)benzoate (2.6 g, 5.9 mmol) in THF (100 mL) was added LiAlH₄ (2.5 M in THF, 3.54 mL), then the mixture was degassed and purged with N₂ three times. The mixture was stirred at 0° C. for 2 hr under N₂ atmosphere. The reaction mixture was quenched with Na₂SO₄·10OH₂O (600 mg) at 0° C. and filtered. The filtrate was concentrated under reduced pressure to give the crude (4-(2-(2-aminopyridin-3-yl)-5-(6-fluoropyridin-3-yl)-3H-imidazo[4,5-b]pyridin-3-yl)phenyl)methanol 2.3 g, yield: 85%) as a yellow solid. MS: m/z=413.0 [M+H]⁺.

### Step 4: 3-(3-(4-(Chloromethyl)phenyl)-5-(6-fluoropyridin-3-yl)-3H-imidazo[4,5-b]pyridin-2-yl)pyridin-2-amine

To a solution of (4-(2-(2-aminopyridin-3-yl)-5-(6-fluoropyridin-3-yl)-3H-imidazo[4,5-b]pyridin-3-yl)phenyl)methanol (2.3 g, 5.58 mmol) in CH₂Cl₂ (50 mL) was added SOCl₂ (3.98 g, 33.5 mmol). The mixture was stirred at 40° C. for 1 hr. The mixture was concentrated under reduced pressure, 3-(3-(4-(chloromethyl)phenyl)-5-(6-fluoropyridin-3-yl)-3H-imidazo[4,5-b]pyridin-2-yl)pyridin-2-amine (Intermediate 131, 2.2 g, yield. 86%) was obtained as a yellow solid. MS: m/z=430.9 [M+H]⁺.

### Intermediate 132: 2-(2-Aminopyridin-3-yl)-3-(4-(chloromethyl)phenyl)-3H-imidazo[4,5-b]pyridine-5-carbonitrile

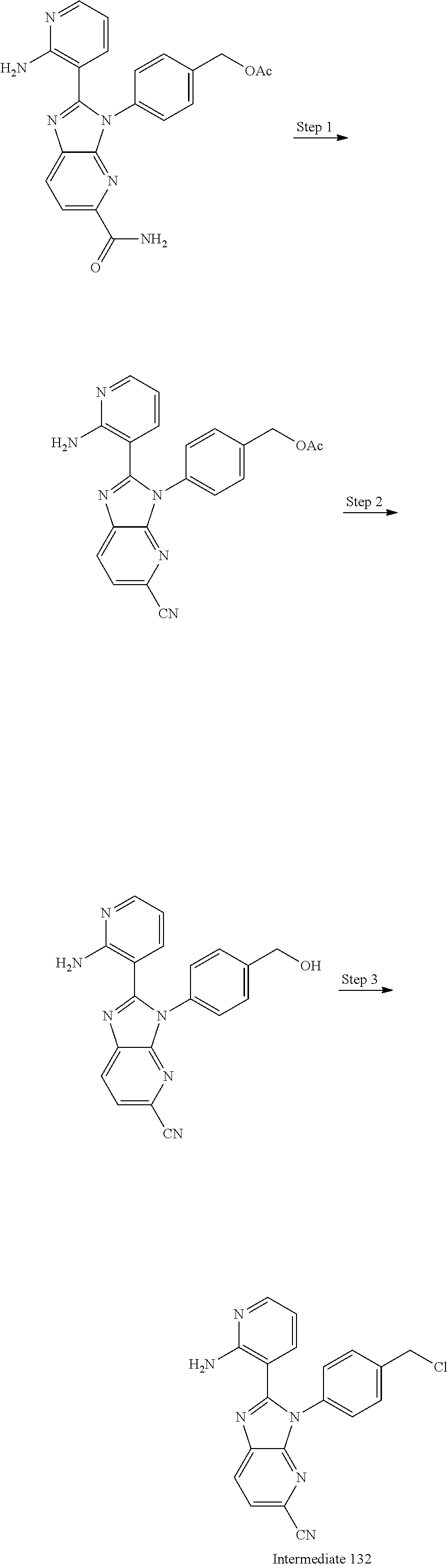 [SMILES: CC(=O)OCc1ccc(-n2c(-c3cccnc3N)nc3ccc(C(N)=O)nc32)cc1 ; CC(=O)OCc1ccc(-n2c(-c3cccnc3N)nc3ccc(C#N)nc32)cc1 ; N#Cc1ccc2nc(-c3cccnc3N)n(-c3ccc(CO)cc3)c2n1 ; N#Cc1ccc2nc(-c3cccnc3N)n(-c3ccc(CCl)cc3)c2n1]

### Step 1: 4-(2-(2-Aminopyridin-3-yl)-5-cyano-3H-imidazo[4,5-b]pyridin-3-yl)benzyl acetate

A solution of 4-(2-(2-aminopyridin-3-yl)-5-carbamoyl-3H-imidazo[4,5-b]pyridin-3-yl)benzyl acetate (refer to Intermediate 74 for detail procedures, 250 mg, 621 μmol) in POCl₃ (3 mL) was stirred at 100° C. for 1 hr. The reaction mixture was filtered and concentrated under reduced pressure to give 4-(2-(2-aminopyridin-3-yl)-5-cyano-3H-imidazo[4,5-b]pyridin-3-yl)benzyl acetate (230 mg, yield: 71%) as a brown solid. MS: m/z=385.0 [M+H]⁺. ¹H NMR (400 MHz, Dimethylsulfoxide-d₆) δ 8.52 (d, J=8.0 Hz, 1H), 8.14-8.07 (m, 2H), 7.95-7.90 (m, 1H), 7.66-7.61 (m, 1H), 7.58-7.44 (m, 5H), 6.91-6.80 (m, 1H), 5.16 (s, 2H), 2.11 (s, 3H).

### Step 2: 2-(2-Aminopyridin-3-yl)-3-(4-(hydroxymethyl)phenyl)-3H-imidazo[4,5-b]pyridine-5-carbonitrile

To a solution of 4-(2-(2-aminopyridin-3-yl)-5-cyano-3H-imidazo[4,5-b]pyridin-3-yl)benzyl acetate (230 mg, 598 μmol) in THF (5 mL) and MeOH (5 mL) was added K₂CO₃ (248 mg, 1.8 mmol). The mixture was stirred at 25° C. for 0.5 hr. The reaction mixture was diluted with H₂O (20 mL) and extracted with CH₂Cl₂ (20 mL×2). The combined organic layers were washed with brine (20 mL), dried over Na₂SO₄, filtered and concentrated under reduced pressure to give 2-(2-aminopyridin-3-yl)-3-(4-(hydroxymethyl)phenyl)-3H-imidazo[4,5-b]pyridine-5-carbonitrile (200 mg, yield: 68%) as a yellow solid. MS: m/z=343.0 [M+H]⁺. ¹H NMR (400 MHz, Dimethylsulfoxide-d₆) δ 8.41 (d, J=8.4 Hz, 1H), 8.04-7.96 (m, 2H), 7.51-7.41 (m, 4H), 7.29 (dd, J=7.6, 1.6 Hz, 1H), 6.96 (s, 2H), 6.43 (dd, J=7.6, 4.8 Hz, 1H), 4.59 (s, 2H).

### Step 3: 2-(2-Aminopyridin-3-yl)-3-(4-(chloromethyl)phenyl)-3H-imidazo[4,5-b]pyridine-5-carbonitrile

To a solution of 2-(2-aminopyridin-3-yl)-3-(4-(hydroxymethyl)phenyl)-3H-imidazo[4,5-b]pyridine-5-carbonitrile (230 mg, 672 μmol) in CH₂Cl₂ (5 mL) was added SOCl₂ (240 mg, 2.02 mmol). The mixture was stirred at 40° C. for 0.5 hr. The reaction was concentrated under reduced pressure to give 2-(2-aminopyridin-3-yl)-3-(4-(chloromethyl)phenyl)-3H-imidazo[4,5-b]pyridine-5-carbonitrile (Intermediate 132, 240 mg, yield: 42%) as a brown solid. MS: m/z=360.9 [M+H]⁺.

### Intermediate 133: 3-(3-(4-(Chloromethyl)phenyl)-5-(methylthio)-3H-imidazo[4,5-b]pyridin-2-yl)pyridin-2-amine

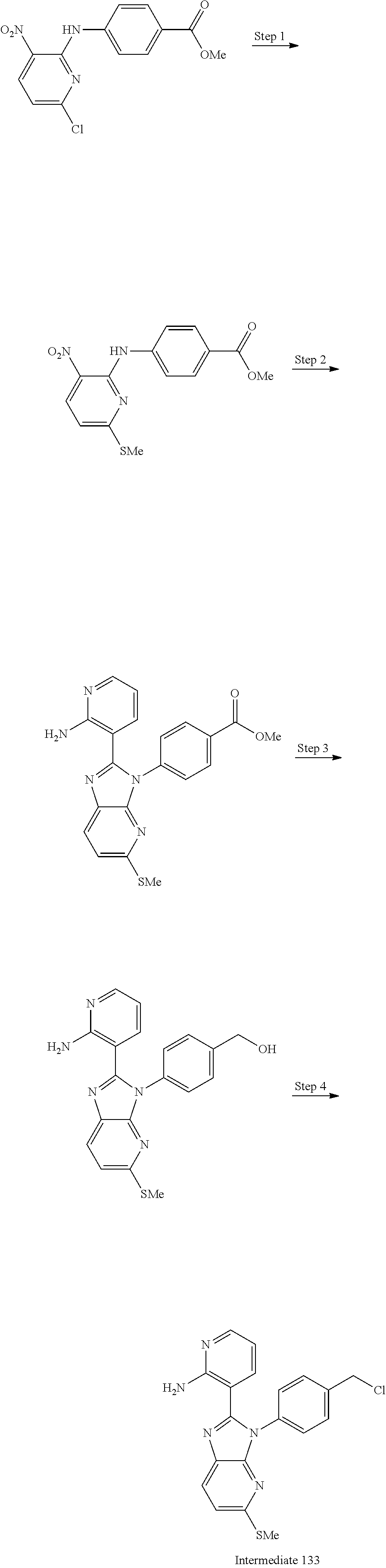 [SMILES: COC(=O)c1ccc(Nc2nc(Cl)ccc2[N+](=O)[O-])cc1 ; COC(=O)c1ccc(Nc2nc(SC)ccc2[N+](=O)[O-])cc1 ; COC(=O)c1ccc(-n2c(-c3cccnc3N)nc3ccc(SC)nc32)cc1 ; CSc1ccc2nc(-c3cccnc3N)n(-c3ccc(CO)cc3)c2n1 ; CSc1ccc2nc(-c3cccnc3N)n(-c3ccc(CCl)cc3)c2n1]

### Step 1: Methyl 4-((6-(methylthio)-3-nitropyridin-2-yl)amino)benzoate

To a solution of methyl 4-[(6-chloro-3-nitro-2-pyridyl)amino]benzoate (refer to Intermediate 13 for detail procedures, 5 g, 16.3 mmol) in THF (50 mL) was added NaSMe (1.09 g, 15.6 mmol). The mixture was stirred at 25° C. for 2 hr. The mixture was diluted with H₂O (50 mL) and extracted with CH₂Cl₂ (100 mL×3). The combined organic layers were washed with brine (50 mL×2), dried over Na₂SO₄, filtered and concentrated under reduced pressure to give methyl 4-((6-(methylthio)-3-nitropyridin-2-yl)amino)benzoate (5 g, yield: 94%), which was used in the next step without further purification. MS: m/z=320.2 [M+H]⁺. ¹H NMR (400 MHz, Chloroform-d) δ 10.63 (s, 1H), 8.31 (d, J=9.2 Hz, 1H), 8.10-8.04 (m, 2H), 7.81-7.73 (m, 2H), 6.76 (d, J=8.8 Hz, 1H), 3.94 (s, 3H), 2.59 (s, 3H).

### Step 2. Methyl 4-(2-(2-aminopyridin-3-yl)-5-(methylthio)-3H-imidazo[4,5-b]pyridin-3-yl)benzoate

To a solution of methyl 4-((6-(methylthio)-3-nitropyridin-2-yl)amino)benzoate (5 g, 15.7 mmol), 2-aminonicotinaldehyde (1.51 g, 15.7 mmol) in DMSO (100 mL) was added Na₂S₂O₄ (10.9 g, 62.6 mmol). The mixture was stirred at 100° C. for 16 hr. The reaction mixture was diluted with H₂O (100 mL) and extracted with CH₂Cl₂ (500 mL×6). The combined organic layers were washed with brine (250 mL×2), dried over Na₂SO₄, filtered and concentrated under reduced pressure. After purified by silica gel flash chromatography (Eluent of 20˜30% EtOAc in CH₂Cl₂), methyl 4-(2-(2-aminopyridin-3-yl)-5-(methylthio)-3H-imidazo[4,5-b]pyridin-3-yl)benzoate (3 g, yield: 44%) was obtained as an orange solid. MS: m/z=392.2 [M+H]⁺. ¹H NMR (400 MHz, Chloroform-d) δ 8.25-8.20 (m, 2H), 7.94-7.92 (m, 2H), 7.51 (d, J=8.4 Hz, 2H), 7.26-7.23 (m, 1H), 7.21-7.11 (m, 1H), 6.49-6.43 (m, 1H), 4.00 (s, 3H), 2.49 (s, 3H).

### Step 3: (4-(2-(2-Aminopyridin-3-yl)-5-(methylthio)-3H-imidazo[4,5-b]pyridin-3-yl)phenyl)methanol

To a solution of methyl 4-(2-(2-aminopyridin-3-yl)-5-(methylthio)-3H-imidazo[4,5-b]pyridin-3-yl)benzoate (1.5 g, 3.83 mmol) in THF (30 mL) was added LiAlH₄ (2.5 M, 1.53 mL) at 0° C., then the mixture was stirred at 25° C. for 2 hr. The reaction mixture was quenched with Na₂SO₄—H₂O (about 1 g) at 0° C., and the mixture was filtered, the filter cake was washed by CH₂Cl₂ (10 mL×3). The filtrate was concentrated under reduced pressure to give (4-(2-(2-aminopyridin-3-yl)-5-(methylthio)-3H-imidazo[4,5-b]pyridin-3-yl)phenyl)methanol (1.2 g, yield: 86%) as a yellow solid, which was used in the next step without purification. MS: m/z=364.1 [M+H]+. ¹H NMR (400 MHz, Methanol-d₄) δ 7.86 (dd, J=5.2 Hz, 1.6 Hz, 1H), 7.53-7.49 (m, 3H), 7.30-7.23 (m, 2H), 6.84 (d, J=8.4 Hz, 1.6 Hz, 1H), 6.67-6.65 (m, 1H), 6.64-6.62 (m, 1H), 4.19 (s, 3H), 2.49 (s, 3H).

### Step 4: 3-(3-(4-(Chloromethyl)phenyl)-5-(methylthio)-3H-imidazo[4,5-b]pyridin-2-yl)pyridin-2-amine

To a solution of (4-(2-(2-aminopyridin-3-yl)-5-(methylthio)-3H-imidazo[4,5-b]pyridin-3-yl)phenyl)methanol (1.2 g, 3.30 mmol) in CH₂Cl₂ (15 mL) was added SOCl₂ (1.18 g, 9.91 mmol) at 0° C. The mixture was stirred at 40° C. for 2 hr. The reaction mixture was quenched with H₂O (0.5 mL) at 0° C., filtered and concentrated under reduced pressure to give 3-(3-(4-(chloromethyl)phenyl)-5-(methylthio)-3H-imidazo[4,5-b]pyridin-2-yl)pyridin-2-amine (Intermediate 133, 500 mg, yield: 42%) as a yellow solid. MS: m/z=382.2 [M+H]⁺.

### Intermediate 134: 2-(2-(2-Aminopyridin-3-yl)-3-(4-(chloromethyl)phenyl)-3H-imidazo[4,5-b]pyridin-5-yl)nicotinonitrile

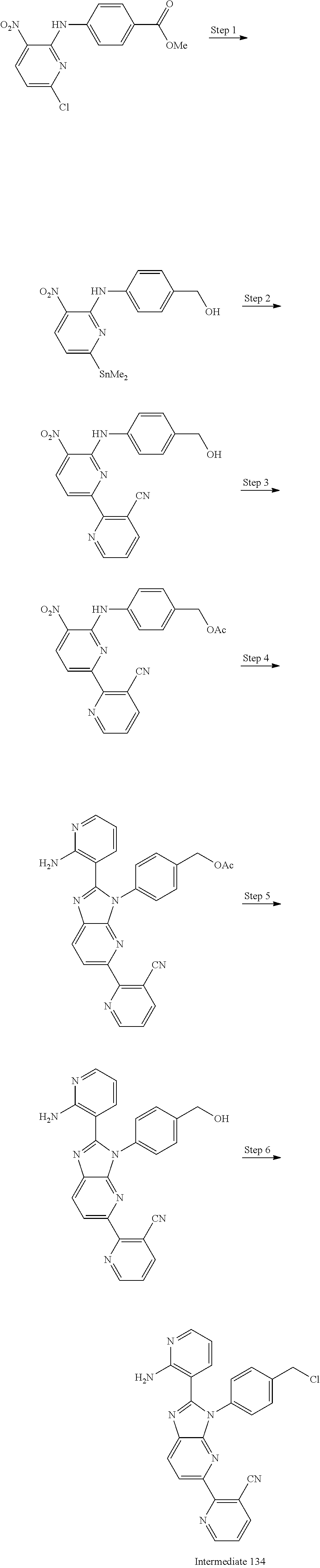

### Step 1: (4-((3-Nitro-6-(trimethylstannyl)pyridin-2-yl)amino)phenyl)methanol

A mixture of methyl 4-((6-chloro-3-nitropyridin-2-yl)amino)benzoate (refer to Intermediate 13 for detail procedures, 4 g, 12.4 mmol), 1,1,1,2,2,2-hexamethyldistannane (8.15 g, 24.9 mmol) and Pd(PPh₃)₄ (229 mg, 622 μmol) in 1,4-dioxane (50 mL) was degassed and purged with N₂ three times, and then the mixture was stirred at 90° C. for 4 hr under N₂ atmosphere. The reaction mixture was concentrated under reduced pressure to give (4-((3-nitro-6-(trimethylstannyl)pyridin-2-yl)amino)phenyl)methanol (5 g, crude) as a black brown solid, which was used in the next step without further purification. MS: m/z=348.0 [M+H]⁺.

### Step 2: 6′-((4-(hydroxymethyl)phenyl)amino)-5′-nitro-[2,2′-bipyridine]-3-carbonitrile

A mixture of (4-((3-nitro-6-(trimethylstannyl)pyridin-2-yl)amino)phenyl)methanol (5 g, 12.3 mmol), 2-bromonicotinonitrile (2.24 g, 12.3 mmol), and Pd(PPh₃)₄ (226 mg, 613 μmol) in 1,4-dioxane (60 mL) was degassed and purged with N₂ three times, and then the mixture was stirred at 100° C. for 16 hr under N₂ atmosphere. The residue was diluted with H₂O (50 mL) and extracted with CH₂Cl₂ (40 mL×3). The combined organic layers were washed with brine (30 mL×2), dried over Na₂SO₄, filtered and concentrated under reduced pressure to give 6′-((4-(hydroxymethyl)phenyl)amino)-5′-nitro-[2,2′-bipyridine]-3-carbonitrile (4.6 g, crude) as an orange red solid, which was directly used in the next step without further purification. MS: m/z=348.0 [M+H]⁺.

### Step 3: 4-((3′-Cyano-5-nitro-[2,2′-bipyridin]-6-yl)amino)benzyl acetate

To a solution of 2-[6-[4-(hydroxymethyl)anilino]-5-nitro-2-pyridyl]pyridine-3-carbonitrile (4 g, 11.5 mmol) and Ac₂O (1.76 g, 17.3 mmol) in CH₂Cl₂ (60 mL) were added DMAP (141 mg, 1.15 mmol) and TEA (3.50 g, 34.6 mmol). The reaction mixture was diluted with H₂O (50 mL) and extracted with CH₂Cl₂ (50 mL×4). The organic layers were washed with brine (50 mL×2), dried over Na₂SO₄, filtered and concentrated under reduced pressure. After purified by silica gel flash chromatography (Eluent of 0˜35% EtOAc in CH₂Cl₂), 4-((3′-cyano-5-nitro-[2,2′-bipyridin]-6-yl)amino)benzyl acetate (1.0 g, yield: 11%) was obtained as a light-yellow solid. MS: m/z=390.0 [M+H]⁺.

### Step 4: 4-(2-(2-Aminopyridin-3-yl)-5-(3-cyanopyridin-2-yl)-3H-imidazo[4,5-b]pyridin-3-yl)benzyl acetate

To a solution of 4-((3′-cyano-5-nitro-[2,2′-bipyridin]-6-yl)amino)benzyl acetate (500 mg, 1.28 mmol) in DMSO (20 mL) were added Na₂S₂O₄ (894 mg, 5.14 mmol, 87% purity) and 2-aminonicotinaldehyde (188 mg, 1.54 mmol). The mixture was stirred at 100° C. for 16 hr. The reaction mixture was quenched with H₂O (25 mL) at 25° C., and then diluted with H₂O (50 mL) and extracted with CH₂Cl₂ (50 mL×3). The combined organic layers were washed with brine (50 mL), dried over Na₂SO₄, filtered and concentrated under reduced pressure. After purified by silica gel flash chromatography (Eluent of 0˜45% EtOAc in petroleum ether), 4-(2-(2-aminopyridin-3-yl)-5-(3-cyanopyridin-2-yl)-3H-imidazo[4,5-b]pyridin-3-yl)benzyl acetate (300 mg, yield: 42%) was obtained as a light-yellow solid. MS: m/z=462.0 [M+H]⁺. ¹H NMR (400 MHz, Chloroform-d) δ 8.82-8.78 (m, 1H), 8.29-8.23 (m, 2H), 8.07-8.04 (m, 2H), 7.60-7.55 (m, 2H), 7.53-7.49 (m, 2H), 7.40-7.36 (m, 1H), 7.22-7.18 (m, 1H), 6.93 (br s, 2H), 6.48-6.37 (m, 1H), 5.18 (s, 2H), 2.13 (s, 3H).

### Step 5: 2-(2-(2-Aminopyridin-3-yl)-3-(4-(hydroxymethyl)phenyl)-3H-imidazo[4,5-b]pyridin-5-yl)nicotinonitrile

To a solution of 4-(2-(2-aminopyridin-3-yl)-5-(3-cyanopyridin-2-yl)-3H-imidazo[4,5-b]pyridin-3-yl)benzyl acetate (300 mg, 650 μmol) in THF (2 mL) and MeOH (2 mL) was added K₂CO₃ (89.9 mg, 650 μmol). The mixture was stirred at 25° C. for 0.5 hr. The reaction mixture was diluted with H₂O (20 mL) and extracted with CH₂Cl₂ (20 mL×2). The combined organic layers were washed with brine (10 mL), dried over Na₂SO₄, filtered and concentrated under reduced pressure to give 2-(2-(2-aminopyridin-3-yl)-3-(4-(hydroxymethyl)phenyl)-3H-imidazo[4,5-b]pyridin-5-yl)nicotinonitrile (280 mg, yield: 82%) as a light-yellow solid. MS: m/z=420.0 [M+H]⁺.

### Step 6: 2-(2-(2-Aminopyridin-3-yl)-3-(4-(chloromethyl)phenyl)-3H-imidazo[4,5-b]pyridin-5-yl)nicotinonitrile

To a solution of 2-(2-(2-aminopyridin-3-yl)-3-(4-(hydroxymethyl)phenyl)-3H-imidazo[4,5-b]pyridin-5-yl)nicotinonitrile (280 mg, 668 μmol) in CH₂Cl₂ (5 mL) was added SOCl₂ (159 mg, 1.34 mmol). The mixture was stirred at 40° C. for 0.5 hr. The reaction was concentrated under reduced pressure to give 2-(2-(2-aminopyridin-3-yl)-3-(4-(chloromethyl)phenyl)-3H-imidazo[4,5-b]pyridin-5-yl)nicotinonitrile (Intermediate 134, 260 mg, yield: 89%) as a yellow solid. MS: m/z=438.1 [M+H]⁺.

### Intermediate 135: 3-(2-(2-Aminopyridin-3-yl)-3-(4-(chloromethyl)phenyl)-3H-imidazo[4,5-b]pyridin-5-yl)picolinonitrile

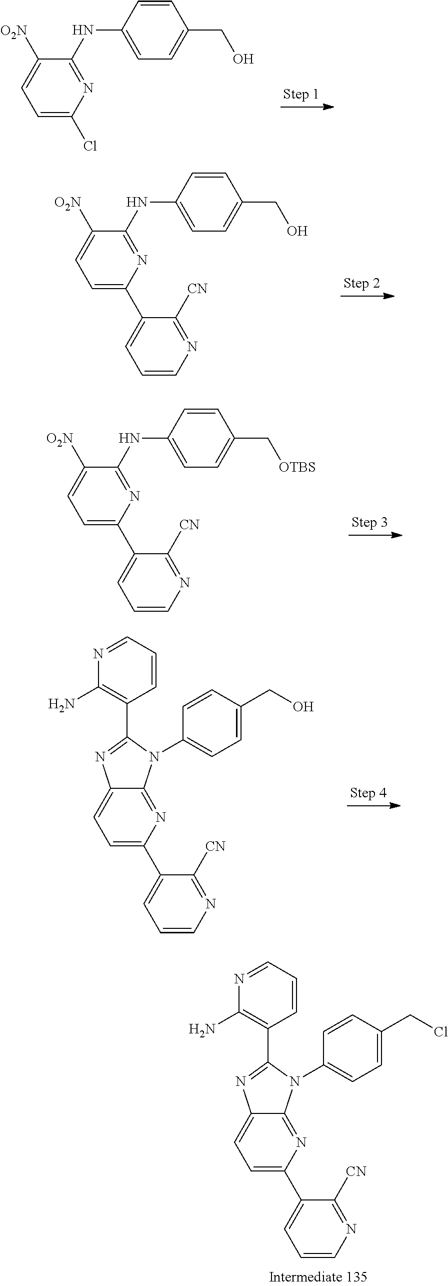

### Step 1: 6-((4-(Hydroxymethyl)phenyl)amino)-5-nitro-[2,3′-bipyridine]-2′-carbonitrile

To a solution of (4-((6-chloro-3-nitropyridin-2-yl)amino)phenyl)methanol (refer to Intermediate 105 for detail procedures, 2.0 g, 7.15 mmol) and (2-cyanopyridin-3-yl)boronic acid (1.16 g, 7.87 mmol) in DMF (20 mL) were added cataCXiumAPdG₃ (2.60 g, 3.58 mmol) and K₃PO₄ (3.04 g, 14.3 mmol) and tricyclohexylphosphane (200 mg, 715 μmol). The mixture was stirred at 120° C. for 3 hr under N₂ atmosphere. The reaction mixture was diluted with H₂O (500 mL) and extracted with EtOAc (250 mL×2). The combined organic layers were washed with brine (500 mL×2), dried over Na₂SO₄, filtered and concentrated under reduced pressure. After purified by silica gel flash chromatography (Eluent of 0˜50% EtOAc in petroleum ether), 6-((4-(hydroxymethyl)phenyl)amino)-5-nitro-[2,3′-bipyridine]-2′-carbonitrile (1.64 g, yield: 63%) was obtained as a black brown solid. MS: m/z=347.9 [M+H]+. ¹H NMR (400 MHz, Dimethylsulfoxide-d₆) δ 10.07 (s, 1H), 8.86 (dd, J=4.8, 1.2 Hz, 1H), 8.74 (d, J=8.4 Hz, 1H), 8.30 (dd, J=8.0, 1.2 Hz, 1H), 7.89 (dd, J=8.0, 4.8 Hz, 1H), 7.67 (d, 1=8.4 Hz, 2H), 7.45 (d, J=8.4 Hz, 1H), 7.29 (d, 1=8.4 Hz, 2H), 5.15 (t, J=5.6 Hz, 1H), 4.47 (d, J=6.0 Hz, 2H)

### Step 2: 6-((4-(((Tert-butyldimethylsilyl)oxy)methyl)phenyl)amino)-5-nitro-[2,3′-bipyridine]-2′-carbonitrile

To a solution of 6-((4-(hydroxymethyl)phenyl)amino)-5-nitro-[2,3′-bipyridine]-2′-carbonitrile (1.6 g, 4.61 mmol) in CH₂Cl₂ (20 mL) were added imidazole (784 mg, 11.5 mmol) and TBSCl (1.39 g, 9.21 mmol) at 0° C. The reaction mixture was stirred at 25° C. for 16 hr. The mixture was concentrated under reduced pressure. After purified by silica gel flash chromatography (Eluent of 0˜35% EtOAc in petroleum ether), 6-((4-(((tert-butyldimethylsilyl)oxy)methyl)phenyl)amino)-5-nitro-[2,3′-bipyridine]-2′-carbonitrile was obtained (1.7 g, yield: 79%) as a red solid. MS: m/z=462.1 [M+H]⁺. ¹H NMR (400 MHz, Dimethylsulfoxide-d₆) δ 10.08 (s, 1H), 8.85 (dd, J=4.8, 1.6 Hz, 1H), 8.74 (d, J=8.4 Hz, 1H), 8.30 (dd, J=8.4, 1.6 Hz, 1H), 7.88 (dd, J=8.0, 4.8 Hz, 1H), 7.68 (d, J=8.4 Hz, 2H), 7.45 (d, J=8.0 Hz, 1H), 7.29 (d, J=8.4 Hz, 2H), 4.68 (s, 2H), 0.89 (s, 9H), 0.07 (s, 6H).

### Step 3: 3-(2-(2-Aminopyridin-3-yl)-3-(4-(hydroxymethyl)phenyl)-3H-imidazo[4,5-b]pyridin-5-yl)picolinonitrile

To a solution of 6-((4-(((tert-butyldimethylsilyl)oxy)methyl)phenyl)amino)-5-nitro-[2,3′-bipyridine]-2′-carbonitrile (1.7 g, 3.68 mmol) and 2-aminonicotinaldehyde (540 mg, 4.42 mmol) in DMSO (50 mL) was added Na₂S₂O₄ (2.56 g, 14.7 mmol). The mixture was stirred at 100° C. for 16 hr. The reaction mixture was diluted with H₂O (300 mL) and extracted with EtOAc (300 mL×2). The combined organic layers were washed with brine (300 mL×2), dried over Na₂SO₄, filtered and concentrated under reduced pressure. After purified by silica gel flash chromatography (Eluent of 0˜5% MeOH in CH₂Cl₂), 3-(2-(2-aminopyridin-3-yl)-3-(4-(hydroxymethyl)phenyl)-3H-imidazo[4,5-b]pyridin-5-yl)picolinonitrile (100 mg, yield: 5.7%) was obtained as a yellow solid. MS: m/z=420.0 [M+H]⁺.

### Step 4: 3-(2-(2-Aminopyridin-3-yl)-3-(4-(chloromethyl)phenyl)-3H-imidazo[4,5-b]pyridin-5-yl)picolinonitrile

To a solution of 3-(2-(2-aminopyridin-3-yl)-3-(4-(hydroxymethyl)phenyl)-3H-imidazo[4,5-b]pyridin-5-yl)picolinonitrile (100 mg, 238 μmol) in CH₂Cl₂ (4 mL) was added SOCl₂ (28.4 mg, 238 μmol). The mixture was stirred at 40° C. for 2 hr. The mixture was concentrated under reduced pressure to give 3-(2-(2-aminopyridin-3-yl)-3-(4-(chloromethyl)phenyl)-3H-imidazo[4,5-b]pyridin-5-yl)picolinonitrile (Intermediate 135, 100 mg, yield: 66%) as a light-yellow solid. MS: m/z=438.0 [M+H]⁺.

### Intermediate 136: 3-(3-(4-(Chloromethyl)phenyl)-5-(trifluoromethyl)-3H-imidazo[4,5-b]pyridin-2-yl)pyridin-2-amine

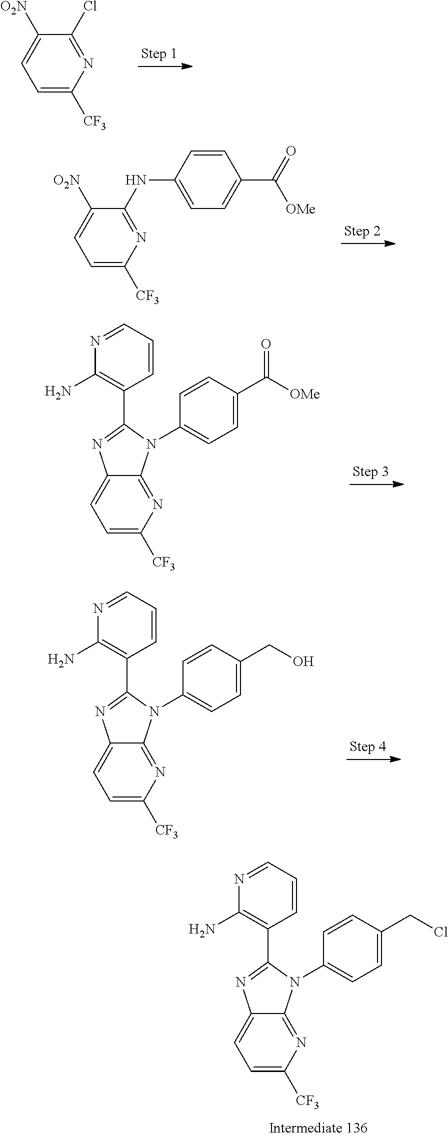

### Step 1; Methyl 4-((3-nitro-6-(trifluoromethyl)pyridin-2-yl)amino)benzoate

To a solution of methyl 2-chloro-3-nitro-6-(trifluoromethyl)pyridine (5 g, 22.1 mmol) in 1,4-dioxane (50 mL) were added DIEA (8.56 g, 66.2 mmol) and methyl 4-aminobenzoate (3.34 g, 22.1 mmol). The mixture was stirred at 100° C. for 16 hr. The reaction mixture was quenched with H₂O (50 mL) at 25° C., and then diluted with H₂O (500 mL) and extracted EtOAc with (200 mL×3). The combined organic layers were washed with brine (50 mL), dried over Na₂SO₄, filtered and concentrated under reduced pressure. The crude product was triturated with EtOAc at 25° C. for 30 min to give methyl 4-((3-nitro-6-(trifluoromethyl)pyridin-2-yl)amino)benzoate (3.7 g, yield: 49%) as a yellow solid. MS: m/z=341.9 [M+H]⁺. ¹H NMR (400 MHz, Dimethylsulfoxide-d₆) δ 10.17 (s, 1H), 8.78 (d, J=8.4 Hz, 1H), 7.96 (d, 1=8.8 Hz, 2H), 7.87-7.80 (m, 2H), 7.51 (d, 0.1=8.4 Hz, 1H), 3.84 (s, 3H). ¹⁹F NMR (400 MHz, Dimethylsulfoxide-d₆) δ −67.72.

### Step 2: Methyl 4-(2-(2-aminopyridin-3-yl)-5-(trifluoromethyl)-3H-imidazo[4,5-b]pyridin-3-yl)benzoate

To a solution of methyl 4-((3-nitro-6-(trifluoromethyl)pyridin-2-yl)amino)benzoate (2.2 g, 6.45 mmol) in DMSO (50 mL) were added Na₂S₂O₄ (4.5 g, 25.8 mmol, 87% purity) and 2-aminonicotinaldehyde (866 mg, 7.1 mmol). The mixture was stirred at 100° C. for 16 hr. The reaction mixture was quenched with H₂O (100 mL) at 25° C., and then diluted with H₂O (300 mL) and extracted with CH₂Cl₂ (100 mL×4). The combined organic layers were washed with brine (50 mL), dried over Na₂SO₄, filtered and concentrated under reduced pressure. The crude was triturated with EtOAc at 25° C. for 30 min to give methyl 4-(2-(2-aminopyridin-3-yl)-5-(trifluoromethyl)-3H-imidazo[4,5-b]pyridin-3-yl)benzoate (1.3 g, yield: 49%) as a yellow solid. MS: m/z=414.0 [M+H]⁺. ¹H NMR (400 MHz, Dimethylsulfoxide-d₆) δ 8.47 (d, J=8.4 Hz, 1H), 8.11 (d, J=8.4 Hz, 2H), 8.03 (dd, J=4.8, 2.0 Hz, 1H), 7.90 (d, J=8.0 Hz, 1H), 7.63 (d, J=8.4 Hz, 2H), 7.30 (dd, J=7.2, 1.2 Hz, 1H), 6.77 (s, 2H), 6.47 (dd, J=7.6, 4.8 Hz, 1H), 3.89 (s, 3H). 19F NMR (400 MHz, Dimethylsulfoxide-d₆) δ −64.28.

### Step 3: (4-(2-(2-Aminopyridin-3-yl)-5-(trifluoromethyl)-3H-imidazo[4,5-b]pyridin-3-yl)phenyl)methanol

To a solution of methyl 4-(2-(2-aminopyridin-3-yl)-5-(trifluoromethyl)-3H-imidazo[4,5-b]pyridin-3-yl)benzoate (500 mg, 1.21 mmol) in THF (5 mL) was added LiAlH₄ (91.8 mg, 2.5 mmol) at 0° C. The resulting mixture was stirred at 25° C. for 1 hr. The reaction mixture was filtered and concentrated to give (4-(2-(2-aminopyridin-3-yl)-5-(trifluoromethyl)-3H-imidazo[4,5-b]pyridin-3-yl)phenyl)methanol (450 mg, yield: 84%) as a brown solid. MS: m/z=388.0 [M+3]⁺.

### Step 4: 3-(3-(4-(Chloromethyl)phenyl)-5-(trifluoromethyl)-3H-imidazo[4,5-b]pyridin-2-yl)pyridin-2-amine

To a solution of (4-(2-(2-aminopyridin-3-yl)-5-(trifluoromethyl)-3H-imidazo[4,5-b]pyridin-3-yl)phenyl)methanol (450 mg, 1.17 mmol) in CH₂Cl₂ (5 mL) was added SOCl₂ (417 mg, 3.5 mmol). The mixture was stirred at 40° C. for 0.5 hr. The reaction was concentrated under reduced pressure to give 3-(3-(4-(chloromethyl)phenyl)-5-(trifluoromethyl)-3H-imidazo[4,5-b]pyridin-2-yl)pyridin-2-amine (Intermediate 136, 450 mg, yield: 68%) was obtained as a brown solid. MS: m/z=404.0 [M+H]⁺.

### Intermediate 137: 2-(2-Aminopyridin-3-yl)-3-(4-(chloromethyl)phenyl)-3H-imidazo[4,5-b]pyridine-6-carboxamide

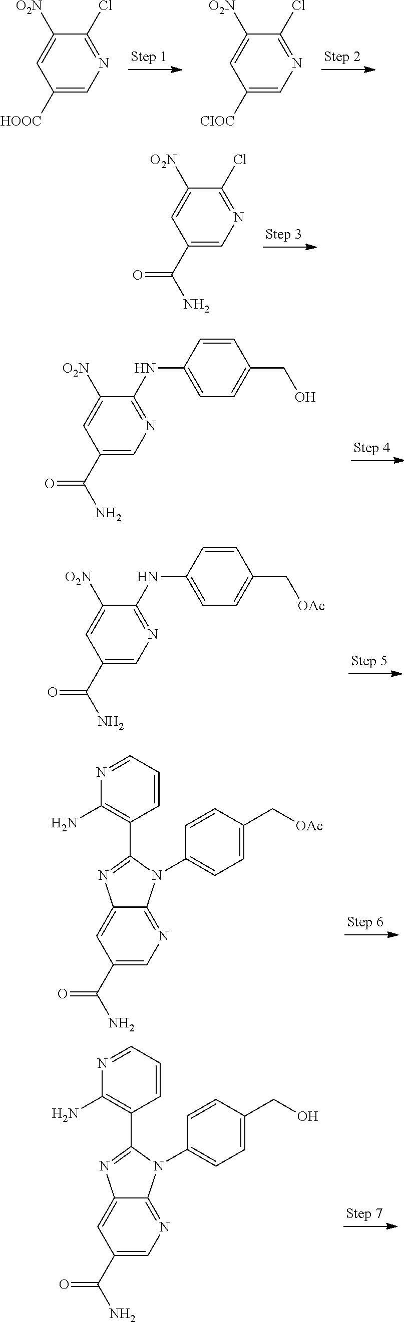

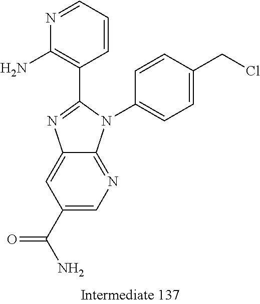 [SMILES: NC(=O)c1cnc2c(c1)nc(-c1cccnc1N)n2-c1ccc(CCl)cc1]

### Step 1: 6-Chloro-5-nitronicotinoyl chloride

To a solution of 6-chloro-5-nitronicotinic acid (5 g, 24.7 mmol) in SOCl₂ (50 mL) was stirred at 80° C. for 16 hr. The reaction mixture was filtered and concentrated under reduced pressure to give 6-chloro-5-nitronicotinoyl chloride (5.4 g, yield: 91%) as a yellow solid. ¹H NMR (400 MHz, Dimethylsulfoxide-d₆) δ 9.12 (s, 1H), 8.87 (s, 1H).

### Step 2: 6-Chloro-5-nitronicotinamide

To a solution of 6-chloro-5-nitronicotinoyl chloride (5.4 g, 24.4 mmol) in THF (50 mL) was added NH₃·H₂O (10.3 g, 73.3 mmol) at 0° C. The mixture was stirred at 0° C. for 1 hr. Then the reaction mixture was diluted with H₂O (100 mL) and extracted with EtOAc (100 mL×2). The combined organic layers were dried over Na₂SO₄, filtered and concentrated under reduced pressure to give 6-chloro-5-nitronicotinamide (2.3 g, yield: 41%) as a yellow solid. MS: m/z=201.9 [M+H]+. ¹H NMR (400 MHz, Dimethylsulfoxide-d₆) δ 9.11 (d, 1=2.0 Hz, 1H), 8.92 (d, J=2.0 Hz, 1H), 8.45 (s, 1H), 7.97 (s, 1H).

### Step 3: 6-((4-(Hydroxymethyl)phenyl)amino)-5-nitronicotinamide

To a solution of 6-chloro-5-nitronicotinamide (2.3 g, 11.4 mmol) in DMSO (40 mL) were added (4-aminophenyl)methanol (1.41 g, 11.4 mmol) and DIEA (4.42 g, 34.2 mmol). The mixture was stirred at 100° C. for 16 hr. The reaction mixture was diluted with H₂O (100 mL) and extracted with CH₂Cl₂ (100 mL×2). The combined organic layers were washed with brine (100 mL×2), dried over Na₂SO₄, filtered and concentrated under reduced pressure to give 6-((4-(hydroxymethyl)phenyl)amino)-5-nitronicotinamide (3.2 g, yield: 78%) as a yellow solid. MS: m/z=289.0 [M+H]⁺. ¹H NMR (400 MHz, Dimethylsulfoxide-d₆) δ 10.17 (s, 1H), 9.63-9.24 (m, 1H), 8.94 (d, J=11.6 Hz, 2H), 8.21 (s, 1H), 7.58-7.55 (m, 2H), 7.33 (d, J=8.0 Hz, 2H), 5.28-5.16 (m, 1H), 4.49 (d, J=4.4 Hz, 2H).

### Step 4: 4-((5-Carbamoyl-3-nitropyridin-2-yl)amino)benzyl acetate

To a solution of 6-((4-(hydroxymethyl)phenyl)amino)-5-nitronicotinamide (3.2 g, 11.1 mmol) in CH₂Cl₂ (20 mL) were added TEA (3.37 g, 33.3 mmol), Ac₂O (1.27 g, 12.5 mmol) and DMAP (1.7 g, 16.7 mmol). The mixture was stirred at 25° C. for 1 hr. The reaction mixture was quenched with H₂O (50 mL) at 25° C., and then diluted with CH₂Cl₂ (200 mL) and washed with H₂O (200 mL×3). The combined organic layers were washed with brine (200 mL), dried over Na₂SO₄, filtered and concentrated under reduced pressure to give 4-((5-carbamoyl-3-nitropyridin-2-yl)amino)benzyl acetate (2.5 g, yield: 84%) as a red solid. MS: m/z=331.0 [M+H]⁺. ¹H NMR (400 MHz, Dimethylsulfoxide-d₆) δ 10.19 (s, 1H), 8.94 (d, J=17.6 Hz, 2H), 8.19 (s, 1H), 7.66-7.53 (m, 3H), 7.39 (d, J=8.0 Hz, 2H), 5.07 (s, 2H), 2.07 (s, 3H).

### Step 5: 4-(2-(2-Aminopyridin-3-yl)-6-carbamoyl-3H-imidazo[4,5-h]pyridin-3-yl)benzyl acetate

To a solution of 4-((5-carbamoyl-3-nitropyridin-2-yl)amino)benzyl acetate (2 g, 6.06 mmol) in DMSO (10 mL) were added Na₂S₂O₄ (2.11 g, 12.1 mmol, 87% purity) and 2-aminonicotinaldehyde (813 mg, 6.66 mmol). The mixture was stirred at 100° C. for 16 hr. The reaction mixture was quenched with H₂O (100 mL) at 25° C., and then diluted with water (200 mL) and extracted with CH₂Cl₂ (100 mL×3). The combined organic layers were washed with brine (200 mL), dried over Na₂SO₄, filtered and concentrated under reduced pressure. After purified by silica gel flash chromatography (Eluent of 5%˜15% MeOH in CH₂Cl₂), 4-(2-(2-aminopyridin-3-yl)-6-carbamoyl-3H-imidazo[4,5-b]pyridin-3-yl)benzyl acetate (400 mg, yield: 12%) was obtained as a brown oil. MS: m/z=403.3 [M+H]+. ¹H NMR (400 MHz, Dimethylsulfoxide-d₆) δ 8.86 (d, J=2.0 Hz, 1H), 8.67 (d, J=2.0 Hz, 1H), 8.14 (s, 1H), 8.04-8.01 (m, 1H), 7.59-7.45 (m, 5H), 7.39-7.33 (m, 1H), 7.05 (br s, 2H), 6.51 (dd, J=7.6, 4.8 Hz, 1H), 5.16 (s, 2H), 2.13 (s, 3H).

### Step 6: 2-(2-Aminopyridin-3-yl)-3-(4-(hydroxymethyl)phenyl)-3H-imidazo[4,5-b]pyridine-6-carboxamide

To a solution of 4-(2-(2-aminopyridin-3-yl)-6-carbamoyl-3H-imidazo[4,5-b]pyridin-3-yl)benzyl acetate (300 mg, 746 μmol) in THF (10 mL) and MeOH (10 mL) was added K₂CO₃ (309 mg, 2.24 mmol). The mixture was stirred at 25° C. for 0.5 hr. The reaction mixture was diluted with H₂O (50 mL) and extracted with CH₂Cl₂ (50 mL×2). The combined organic layers were washed with brine (50 mL), dried over Na₂SO₄, filtered and concentrated under reduced pressure to give 2-(2-aminopyridin-3-yl)-3-(4-(hydroxymethyl)phenyl)-3H-imidazo[4,5-b]pyridine-6-carboxamide (260 mg, yield: 83%) as a yellow solid. MS: m/z=361.2 [M+H]⁺. ¹H NMR (400 MHz, Dimethylsulfoxide-d₆) δ 8.93 (d, J=2.0 Hz, 1H), 8.75 (d, J=2.0 Hz, 1H), 8.58-8.28 (m, 2H), 8.20-8.07 (m, 1H), 7.95 (dd, J=7.6, 1.6 Hz, 1H), 7.60 (s, 1H), 7.47-7.46 (m, 4H), 6.96-6.82 (m, 1H), 4.57 (s, 2H).

### Step 7: 2-(2-Aminopyridin-3-yl)-3-(4-(chloromethyl)phenyl)-3H-imidazo[4,5-b]pyridine-6-carboxamide

To a solution of 2-(2-aminopyridin-3-yl)-3-(4-(hydroxymethyl)phenyl)-3H-imidazo[4,5-b]pyridine-6-carboxamide (260 mg, 721 μmol) in CH₂Cl₂ (5 mL) was added SOCl₂ (258 mg, 2.16 mmol). The mixture was stirred at 40° C. for 0.5 hr. The reaction mixture was concentrated under reduced pressure to give 2-(2-aminopyridin-3-yl)-3-(4-(chloromethyl)phenyl)-3H-imidazo[4,5-b]pyridine-6-carboxamide (Intermediate 137, 270 mg, yield: 74%) as a yellow solid. MS: m/z=379.2 [M+H]⁺.

### Intermediate 138: 3-(3-(4-(Chloromethyl)phenyl)-5,6-dimethyl-3H-imidazo[4,5-b]pyridin-2-yl)pyridin-2-amine

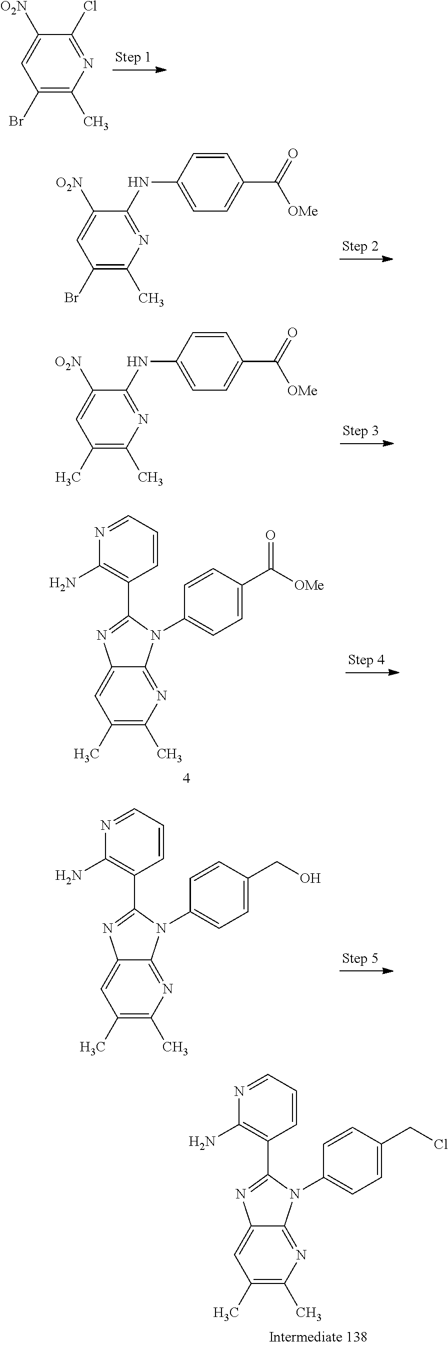

### Step 1: Methyl 4-((5-bromo-6-methyl-3-nitropyridin-2-yl)amino)benzoate

To a solution of 3-bromo-6-chloro-2-methyl-5-nitropyridine (8 g, 31.8 mmol) and methyl 4-aminobenzoate (5.29 g, 35 mmol) in DMSO (200 mL) was added DIEA (4.93 g, 38.2 mmol). The mixture was stirred at 100° C. for 16 hr. The mixture was added into H₂O (500 mL), the mixture was filtered and the filter cake was concentrated under reduced pressure. After purified by silica gel flash chromatography (Eluent of 0˜50% EtOAc in petroleum ether (adding 50% CH₂Cl₂)) and purified again by triturated with EtOAc (20 mL) at 25° C. for 0.5 hr, methyl 4-((5-bromo-6-methyl-3-nitropyridin-2-yl)amino)benzoate (2.2 g, yield: 17%) was obtained as a red solid. MS: m/z=365.8, 367.8 [M+H]^{+ 1}H NMR (400 MHz, Dimethylsulfoxide-d₆) δ 10.11 (s, 1H), 8.66 (s, 1H), 7.95 (d, J=8.8 Hz, 2H), 7.87 (d, J=8.8 Hz, 2H), 3.84 (s, 3H), 2.59 (s, 3H).

### Step 2: Methyl 4-((5,6-dimethyl-3-nitropyridin-2-yl)amino)benzoate

To a solution of methyl 4-((5-bromo-6-methyl-3-nitropyridin-2-yl)amino)benzoate (2.1 g, 5.74 mmol) and methylboronic acid (412 mg, 6.88 mmol) in H₂O (2 mL) and 1,4-dioxane (20 mL) were added Pd(dppf)Cl₂ (41.9 mg, 57.4 μmol) and Cs₂CO₃ (3.74 g, 11.5 mmol). The mixture was stirred at 100° C. for 16 hr under N₂ atmosphere. The mixture was concentrated under reduced pressure. After purified by silica gel flash chromatography (Eluent of 0˜10% EtOAc in petroleum ether (adding 20% CH₂Cl₂)), methyl 4-((5,6-dimethyl-3-nitropyridin-2-yl)amino)benzoate (1.6 g, yield: 81%) was obtained as a red solid. MS: m/z=302.0 [M+H]⁺. ¹H NMR (400 MHz, Dimethylsulfoxide-d₆) 610.10 (s, 1H), 8.34 (s, 1H), 7.93 (br s, 4H), 3.83 (s, 3H), 2.48 (s, 3H), 2.27 (s, 3H).

### Step 3: Methyl 4-(2-(2-aminopyridin-3-yl)-5,6-dimethyl-3H-imidazo[4,5-h]pyridin-3-yl)benzoate

To a solution of methyl 4-((5,6-dimethyl-3-nitropyridin-2-yl)amino)benzoate (1.5 g, 4.98 mmol) and 2-aminonicotinaldehyde (669 mg, 5.48 mmol) in DMSO (30 mL) was added Na₂S₂O₄ (3.47 g, 19.9 mmol). The mixture was stirred at 100° C. for 16 hr. H₂O (50 mL) was added to the mixture. The mixture was filtered. The filter cake was dissolved in CH₂Cl₂ (200 mL). The organic layer was washed with brine (100 mL×2), dried over Na₂SO₄, filtered and concentrated under reduced pressure to give a yellow solid. The yellow solid was triturated with EtOAc (10 mL) at 25° C. for 30 min to give methyl 4-(2-(2-aminopyridin-3-yl)-5,6-dimethyl-3H-imidazo[4,5-b]pyridin-3-yl)benzoate (950 mg, yield: 66%) as a light-yellow solid. MS: m/z=374.1 [M+H]⁺. ¹H NMR (400 MHz, Dimethylsulfoxide-d₆) 68.08 (d, J=8.4 Hz, 2H), 7.99-7.96 (m, 2H), 7.55 (d, J=8.8 Hz, 2H), 7.12 (dd, J=7.6, 1.6 Hz, 1H), 6.91 (s, 2H), 6.40 (dd, J=7.6, 4.8 Hz, 1H), 3.89 (s, 3H), 2.45 (s, 3H), 2.39 (s, 3H).

### Step 4: (4-(2-(2-Aminopyridin-3-yl)-5,6-dimethyl-3H-imidazo[4,5-b]pyridin-3-yl)phenyl)methanol

To a solution of methyl 4-(2-(2-aminopyridin-3-yl)-5,6-dimethyl-3H-imidazo[4,5-b]pyridin-3-yl)benzoate (950 mg, 2.54 mmol) in THF (10 mL) was added LiAlH₄ (2.5 M, 2.04 mL) at 0° C. The mixture was stirred at 25° C. for 0.5 hr under N₂ atmosphere. The Na₂SO₄·10H₂O (1 g) was added into the mixture slowly at 0° C. The mixture was stirred at 25° C. for 20 min. The mixture was filtered, concentrated under reduced pressure to give (4-(2-(2-aminopyridin-3-yl)-5,6-dimethyl-3H-imidazo[4,5-b]pyridin-3-yl)phenyl)methanol (870 mg, yield: 55%) as a light-yellow solid. MS: m/z=346.0 [M+H]⁺.

### Step 5: 3-(3-(4-(Chloromethyl)phenyl)-5,6-dimethyl-3H-imidazo[4,5-b]pyridin-2-yl)pyridin-2-amine

To a solution of (4-(2-(2-aminopyridin-3-yl)-5,6-dimethyl-3H-imidazo[4,5-b]pyridin-3-yl)phenyl)methanol (870 mg, 2.52 mmol) in CH₂Cl₂ (10 mL) was added SOCl₂ (819 mg, 6.88 mmol). The mixture was stirred at 40° C. for 2 hr. The mixture was concentrated under reduced pressure to give 3-(3-(4-(chloromethyl)phenyl)-5,6-dimethyl-3H-imidazo[4,5-b]pyridin-2-yl)pyridin-2-amine (Intermediate 138, 1 g, yield: 87%) as a yellow solid. MS: m/z=364.0 [M+H]⁺.

### Intermediate 139: 3-(1-(4-(Chloromethyl)phenyl)-1H-imidazo[4,5-b]pyrazin-2-yl)pyridin-2-amine

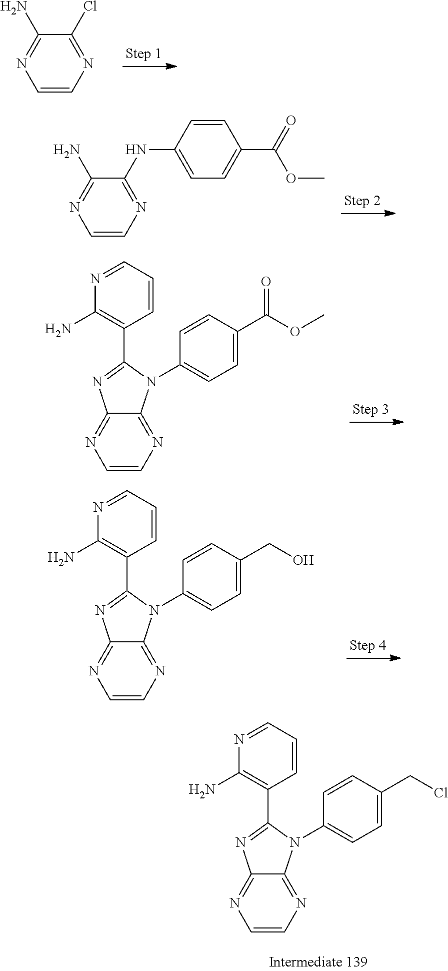

### Step 1; Methyl 4-((3-aminopyrazin-2-yl)amino)benzoate

To a mixture of 3-chloropyrazin-2-amine (5 g, 38.6 mmol) in H₂O (100 mL) were added TsOH·H₂O (7.34 g, 38.6 mmol) and methyl 4-aminobenzoate (5.83 g, 38.6 mmol). The mixture was stirred at 100° C. for 16 hr. The reaction mixture was filtered and concentrated under reduced pressure. The crude was triturated with EtOAc/MeOH (1:1) (50 mL) to give methyl 4-((3-aminopyrazin-2-yl)amino)benzoate (10 g, yield: 77%) as an off-white solid. MS: m/z=245.1 [M+H]⁺. ¹H NMR (400 MHz, Dimethylsulfoxide-d₆) δ 9.31 (s, 1H), 7.58 (d, J=3.6 Hz, 1H), 7.49 (d, J=8.0 Hz, 2H), 7.44 (d, J=14.0 Hz, 1H), 7.12 (d, J=8.0 Hz, 2H), 3.83 (s, 3H).

### Step 2: Methyl 4-(2-(2-aminopyridin-3-yl)-1H-imidazo[4,5-h]pyrazin-1-yl)benzoate

A mixture of methyl 4-((3-aminopyrazin-2-yl)amino)benzoate (5 g, 20.4 mmol), 2-aminonicotinaldehyde (2.75 g, 22.5 mmol), and Cu(OAc)₂ (743 mg, 4.09 mmol) in AcOH (300 mL) was degassed and purged with N₂ three times. The mixture was stirred at 70° C. for 24 hr under N₂ atmosphere. The reaction mixture was diluted with H₂O (100 mL) and pH was adjusted to about 7 with sat. Na₂CO₃ (1500 mL). The mixture was extracted with EtOAc (300 mL). The combined organic layers were washed with H₂O (100 mL×3), dried over Na₂SO₄, filtered and concentrated under reduced pressure. After purified by silica gel flash chromatography (Eluent of 0˜50% EtOAc in petroleum ether), methyl 4-(2-(2-aminopyridin-3-yl)-1H-imidazo[4,5-h]pyrazin-1-yl)benzoate (1.6 g, yield: 22%) was obtained as a yellow solid. MS: m/z=347.0 [M+H]⁺. ¹H NMR (400 MHz, Dimethylsulfoxide-d₆) δ 8.61 (d, J=2.8 Hz, 1H), 8.40 (d, J=2.8 Hz, 1H), 8.12-8.07 (m, 2H), 8.05 (dd, 1=4.8, 2.0 Hz, 1H), 7.66-7.59 (m, 2H), 7.34 (dd, 1=8.0, 2.0 Hz, 1H), 6.80 (s, 2H), 6.48 (dd, J=8.0, 5.2 Hz, 1H), 3.89 (s, 3H).

### Step 3: (4-(2-(2-Aminopyridin-3-yl)-1H-imidazo[4,5-b]pyrazin-1-yl)phenyl)methanol

A mixture of methyl 4-(2-(2-aminopyridin-3-yl)-1H-imidazo[4,5-b]pyrazin-1-yl)benzoate (1 g, 2.89 mmol) in THF (100 mL) was added LiAlH₄ (2.5 M, 1.73 mL) dropwise at 0° C. The mixture was stirred at 25° C. for 1 hr under N₂ atmosphere. The reaction mixture was quenched with 1.7 g Na₂SO₄·10H₂O at 0° C., filtered and concentrated under reduced pressure to give (4-(2-(2-aminopyridin-3-yl)-1H-imidazo[4,5-b]pyrazin-1-yl)phenyl)methanol (750 mg, crude) as a yellow solid. MS: m/z=318.9 [M+H]⁺.

### Step 4: 3-(1-(4-(Chloromethyl)phenyl)-1H-imidazo[4,5-b]pyrazin-2-yl)pyridin-2-amine

A mixture of (4-(2-(2-aminopyridin-3-yl)-1H-imidazo[4,5-b]pyrazin-1-yl)phenyl)methanol (745 mg, 2.34 mmol) in CH₂Cl₂ (5 mL) was added SOCl₂ (1.67 g, 14.0 mmol), and then the mixture was stirred at 40° C. for 1 hr. The reaction mixture was filtered and concentrated under reduced pressure to give 3-(1-(4-(chloromethyl)phenyl)-1H-imidazo[4,5-b]pyrazin-2-yl)pyridin-2-amine (Intermediate 139, 100 mg, yield: 10% for 2 steps) as a brown solid. MS: m/z=337.0 [M+H]⁺. ¹H NMR (400 MHz, Dimethylsulfoxide-d₆) δ 8.69 (d, J=2.8 Hz, 1H), 8.50 (d, J=2.8 Hz, 1H), 8.15 (dd, J=6.0, 1.2 Hz, 1H), 7.96 (dd, J=7.6, 1.2 Hz, 1H), 7.62 (d, J=8.4 Hz, 2H), 7.54 (d, J=8.4 Hz, 2H), 6.90 (dd, J=7.2, 6.0 Hz, 1H), 4.84 (s, 2H).

### Intermediate 140: Methyl 2-(2-aminopyridin-3-yl)-3-(4-(chloromethyl)phenyl)-3H-imidazo[4,5-b]pyridine-5-carboxylate

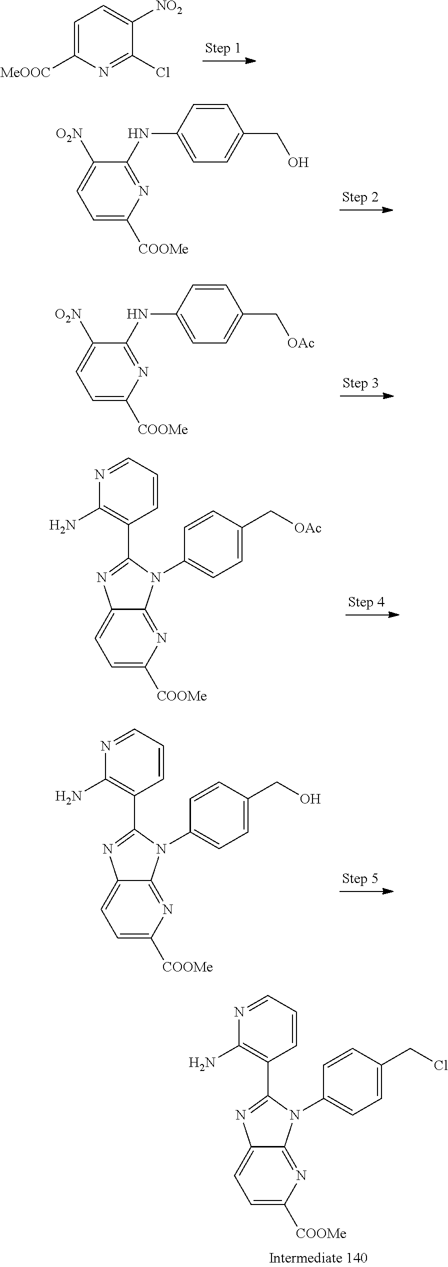

### Step 1: Methyl 6-((4-(hydroxymethyl)phenyl)amino)-5-nitropicolinate

To a solution of 6-chloro-5-nitropyridin-2-yl acetate (4.8 g, 22.2 mmol) in DMSO (40 mL) were added (4-aminophenyl)methanol (2.73 g, 22.2 mmol) and DIEA (8.59 g, 66.5 mmol). The mixture was stirred at 100° C. for 1 hr. The reaction mixture was diluted with H₂O (200 mL) and extracted with CH₂Cl₂ (200 mL×2). The combined organic layers were washed with brine (200 mL×2), dried over Na₂SO₄, filtered and concentrated under reduced pressure to give methyl 6-((4-(hydroxymethyl)phenyl)amino)-5-nitropicolinate (6 g, yield: 74%) as a yellow solid. MS: m/z=304.0 [M+H]f ¹H NMR (400 MHz, Dimethylsulfoxide-d₆) δ 9.95 (s, 1H), 9.32 (br s, 1H), 8.68 (d, J=8.4 Hz, 1H), 7.78 (d, J=8.4 Hz, 2H), 7.52 (d, J=8.4 Hz, 1H), 7.31 (d, J=8.0 Hz, 2H), 5.18 (t, J=5.6 Hz, 2H), 4.49 (d, J=5.2 Hz, 2H), 3.90 (s, 3H).

### Step 2: Methyl 6-((4-(acetoxymethyl)phenyl)amino)-5-nitropicolinate

To a solution of methyl 6-((4-(hydroxymethyl)phenyl)amino)-5-nitropicolinate (6 g, 19.8 mmol) in CH₂Cl₂ (20 mL) were added TEA (6.01 g, 59.4 mmol), Ac₂O (3.03 g, 29.7 mmol) and DMAP (242 mg, 1.98 mmol). The mixture was stirred at 25° C. for 1 hr. The reaction mixture was quenched with H₂O (100 mL) at 25° C., diluted with water (200 mL) and extracted with CH₂Cl₂ (200 mL×3). The combined organic layers were washed with brine (200 mL), dried over Na₂SO₄, filtered and concentrated under reduced pressure to give methyl 6-((4-(acetoxymethyl)phenyl)amino)-5-nitropicolinate (6.5 g, yield: 59%) as a red solid. MS: m/z=346.0 [M+H]⁺. ¹H NMR (400 MHz, Dimethylsulfoxide-d₆) δ 9.97 (s, 1H), 8.68 (d, J=8.4 Hz, 1H), 7.84 (d, J=8.4 Hz, 2H), 7.54 (d, J=8.4 Hz, 1H), 7.37 (d, J=8.4 Hz, 2H), 5.06 (s, 2H), 3.91 (s, 3H), 2.07 (s, 3H).

### Step 3: Methyl 3-(4-(acetoxymethyl)phenyl)-2-(2-aminopyridin-3-yl)-3H-imidazo[4,5-b]pyridine-5-carboxylate

To a solution of methyl 6-((4-(acetoxymethyl)phenyl)amino)-5-nitropicolinate (4 g, 11.6 mmol) in DMSO (20 mL) were added Na₂S₂O₄ (4.03 g, 23.2 mmol, 87% purity) and 2-aminonicotinaldehyde (1.56 g, 12.7 mmol). The mixture was stirred at 100° C. for 16 hr. The reaction mixture was quenched with H₂O (100 mL) at 25° C., and then diluted with H₂O (200 mL) and extracted with CH₂Cl₂ (100 mL×2). The combined organic layers were washed with brine (200 mL), dried over Na₂SO₄, filtered and concentrated under reduced pressure. After purified by silica gel flash chromatography (Eluent of 30˜50%, EtOAc in petroleum ether), methyl 3-(4-(acetoxymethyl)phenyl)-2-(2-aminopyridin-3-yl)-3H-imidazo[4,5-b]pyridine-5-carboxylate (560 mg, yield: 10%) was obtained as a yellow solid. MS: m/z=418.0 [M+H]⁺. ¹H NMR (400 MHz, Dimethylsulfoxide-d₆) δ 8.34 (d, J=8.0 Hz, 1H), 8.12 (d, J=8.0 Hz, 1H), 8.03-7.98 (m, 1H), 7.57-7.44 (m, 4H), 7.25 (dd, J=7.6, 1.6 Hz, 1H), 6.87 (s, 2H), 6.51-6.34 (m, 1H), 5.17 (s, 2H), 3.85 (s, 3H), 2.12 (s, 3H).

### Step 4: Methyl 2-(2-aminopyridin-3-yl)-3-(4-(hydroxymethyl)phenyl)-3H-imidazo[4,5-b]pyridine-5-carboxylate

To a solution of methyl 3-(4-(acetoxymethyl)phenyl)-2-(2-aminopyridin-3-yl)-3H-imidazo[4,5-b]pyridine-5-carboxylate (560 mg, 1.34 mmol) in THF (10 mL) and MeOH (10 mL) was added K₂CO₃ (556 mg, 4.02 mmol). The mixture was stirred at 25° C. for 0.5 hr. The reaction mixture was diluted with H₂O (100 mL) and extracted with CH₂Cl₂ (100 mL×2). The combined organic layers were washed with brine (100 mL), dried over Na₂SO₄, filtered and concentrated under reduced pressure to give methyl 2-(2-aminopyridin-3-yl)-3-(4-(hydroxymethyl)phenyl)-3H-imidazo[4,5-b]pyridine-5-carboxylate (330 mg, yield: 57%) was obtained as a yellow solid. MS: m/z=376.0 [M+H]+. ¹H NMR (400 MHz, Dimethylsulfoxide-d₆) δ 8.33 (d, J=8.0 Hz, 1H), 8.12 (d, J=8.4 Hz, 1H), 8.03-7.98 (m, 1H), 7.51-7.39 (m, 4H), 7.28-7.20 (m, 1H), 6.94 (s, 2H), 6.42 (dd, J=7.2, 4.8 Hz, 1H), 5.46-5.29 (m, 1H), 4.59 (d, J=4.4 Hz, 2H), 3.85 (s, 3H).

### Step 5: Methyl 2-(2-aminopyridin-3-yl)-3-(4-(chloromethyl)phenyl)-3H-imidazo[4,5-b]pyridine-5-carboxylate

To a solution of methyl 2-(2-aminopyridin-3-yl)-3-(4-(hydroxymethyl)phenyl)-3H-imidazo[4,5-b]pyridine-5-carboxylate (330 mg, 879 μmol) in CH₂Cl₂ (5 mL) was added SOCl₂ (314 mg, 2.64 mmol). The mixture was stirred at 40° C. for 0.5 hr. The reaction was concentrated under reduced pressure to give methyl 2-(2-aminopyridin-3-yl)-3-(4-(chloromethyl)phenyl)-3H-imidazo[4,5-b]pyridine-5-carboxylate (Intermediate 140, 340 mg, yield: 98%) as a yellow solid. MS: m/z=394.1 [M+H]⁺.

### Intermediate 141: 3-(3-(4-(Chloromethyl)phenyl)-5-(fluoromethoxy)-3H-imidazo[4,5-b]pyridin-2-yl)pyridin-2-amine

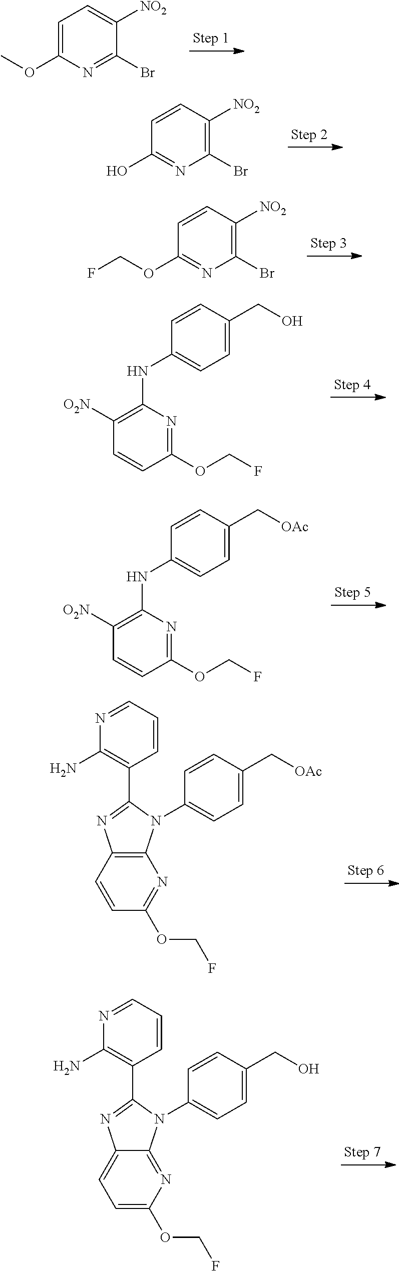

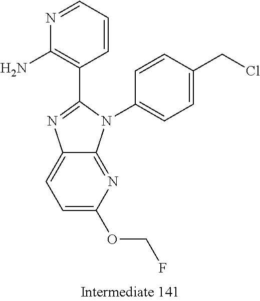 [SMILES: Nc1ncccc1-c1nc2ccc(OCF)nc2n1-c1ccc(CCl)cc1]

### Step 1: 6-Bromo-5-nitropyridin-2-ol

A solution of 2-bromo-6-methoxy-3-nitropyridine (9.9 g, 42.5 mmol) in hydrogen bromide (186 g, 759 mmol, 33% purity) was stirred at 80° C. for 12 hr. The reaction mixture was filtered and washed with petroleum ether. The filter cake was concentrated under reduced pressure to give 6-bromo-5-nitropyridin-2-ol (7.4 g, yield: 71%) as a brown solid. ¹H NMR (400 MHz, Dimethylsulfoxide-d₆) δ 14.90-11.14 (m, 1H), 8.35 (d, J=8.8 Hz, 1H), 6.79 (d, J=8.8 Hz, 1H).

### Step 2: 2-Bromo-6-(fluoromethoxy)-3-nitropyridine

To a solution of 6-bromo-5-nitropyridin-2-ol (4 g, 18.3 mmol) in DMF (30 mL) were added K₂CO₃ (5.05 g, 36.5 mmol) and CH₂FI (4.38 g, 27.4 mmol). The mixture was stirred at 20° C. for 2 hr. The reaction mixture was diluted with water (50 mL) and extracted with EtOAc (2×30 mL). The combined organic layers were washed with brine (130 mL), dried over Na₂SO₄, filtered and concentrated under reduced pressure. 2-bromo-6-(fluoromethoxy)-3-nitropyridine (3.1 g, yield: 64%) was obtained as a yellow solid. ¹H NMR (400 MHz, Dimethylsulfoxide-d₆) δ 8.55 (d, J=8.8 Hz, 1H), 7.28 (d, J=8.8 Hz, 1H), 6.11 (d, J=51.6 Hz, 2H), ¹⁹F NMR (400 MHz, Dimethylsulfoxide-d₆) 6-156.386.

### Step 3: (4-((6-(Fluoromethoxy)-3-nitropyridin-2-yl)amino)phenyl)methanol

To a solution of 2-bromo-6-(fluoromethoxy)-3-nitropyridine (3.1 g, 12.3 mmol) and (4-aminophenyl)methanol (1.67 g, 13.6 mmol) in DMSO (30 mL) was added DIEA (4.79 g, 37.1 mmol). The mixture was stirred at 100° C. for 2 hr. The reaction mixture was diluted with water (50 mL), filtered and concentrated under reduced pressure to give (4-((6-(fluoromethoxy)-3-nitropyridin-2-yl)amino)phenyl)methanol (3.6 g, crude) as yellow a solid. MS: m/z=294.1 [M+H]⁺.

### Step 4: 4-((6-(Fluoromethoxy)-3-nitropyridin-2-yl)amino)benzyl acetate

To a solution of 4-((6-(fluoromethoxy)-3-nitropyridin-2-yl)amino)benzyl acetate (3.5 g, 11.9 mmol) in CH₂Cl₂ (30 mL) were added Ac₂O (1.22 g, 11.9 mmol), DMAP (145 mg, 1.19 mmol) and TEA (3.62 g, 35.8 mmol). The mixture was stirred at 0° C. for 2 hr. The reaction mixture was diluted with water (20 mL) and extracted with CH₂Cl₂ (3×30 mL). The combined organic layers were washed with brine (30 mL), dried over Na₂SO₄, filtered and concentrated under reduced pressure. After purified by silica gel flash chromatography (Eluent of 0˜100% EtOAc in petroleum ether), 4-((6-(fluoromethoxy)-3-nitropyridin-2-yl)amino)benzyl acetate (3.2 g, yield: 79%) was obtained as yellow solid. MS: m/z=336.1 [M+H]⁺. ¹H NMR (400 MHz, Dimethylsulfoxide-d₆) δ 10.35 (s, 1H), 8.58 (d, J=8.8 Hz, 1H), 7.68 (d, J=8.4 Hz, 2H), 7.38 (d, J=8.4 Hz, 2H), 6.53 (d, J=9.2 Hz, 1H), 6.01 (d, J=51.6 Hz, 2H), 5.07 (s, 2H), 2.07 (s, 3H), ¹⁹F NMR (400 MHz, Dimethylsulfoxide-d₆) δ −156.402.

### Step 5: 4-(2-(2-Aminopyridin-3-yl)-5-(fluoromethoxy)-3H-imidazo[4,5-b]pyridin-3-yl)benzyl acetate

To a solution of 4-((6-(fluoromethoxy)-3-nitropyridin-2-yl)amino)benzyl acetate (3.2 g, 9.54 mmol) and 2-aminonicotinaldehyde (1.28 g, 10.5 mmol) in DMSO (100 mL) was added Na₂S₂O₄ (7.64 g, 38.2 mmol, 85% purity). The mixture was stirred at 100° C. for 12 hr. The reaction mixture was diluted with water (100 mL) and extracted with CH₂Cl₂ (300 mL×2). The combined organic layers were washed with brine (100 mL), dried over Na₂SO₄, filtered and concentrated under reduced pressure. The residue was purified by silica gel flash chromatography (Eluent of 0˜100% EtOAc in petroleum ether) to give 4-(2-(2-aminopyridin-3-yl)-5-(fluoromethoxy)-3H-imidazo[4,5-b]pyridin-3-yl)benzyl acetate (300 mg, yield: 6.8%) as a yellow solid. MS: m/z=408.0 [M+H]⁺. ¹H NMR (400 MHz, Dimethylsulfoxide-d₆) δ 8.25 (d, J=8.4 Hz, 1H), 7.98 (dd, J=4.8, 1.6 Hz, 1H), 7.53-7.48 (m, 2H), 7.45-7.42 (m, 2H), 7.17 (dd, J=7.6, 2.0 Hz, 1H), 6.98 (d, J=8.4 Hz, 1H), 6.78 (br s, 2H), 6.42 (dd, J=7.6, 4.8 Hz, 1H), 6.10-6.05 (m, 1H), 5.96-5.92 (m, 1H), 5.15 (s, 2H), 2.11 (s, 3H), ¹⁹F NMR (400 MHz, Dimethylsulfoxide-d₆) δ −153.980.

### Step 6: (4-(2-(2-Aminopyridin-3-yl)-5-(fluoromethoxy)-3H-imidazo[4,5-b]pyridin-3-yl)phenyl)methanol

To a solution of 4-(2-(2-aminopyridin-3-yl)-5-(fluoromethoxy)-3H-imidazo[4,5-b]pyridin-3-yl)benzyl acetate (300 mg, 736 μmol) in MeOH (3 mL) and THF (3 mL) was added a solution of K₂CO₃ (101 mg, 736 μmol) in H₂O (1 mL). The mixture was stirred at 25° C. for 0.5 hr. The reaction mixture was diluted with water (10 mL) and extracted with EtOAc (2×10 mL). The combined organic layers were dried over Na₂SO₄, filtered and concentrated under reduced pressure to give (4-(2-(2-aminopyridin-3-yl)-5-(fluoromethoxy)-3H-imidazo[4,5-b]pyridin-3-yl)phenyl)methanol (260 mg, yield: 83%) as a pink solid. MS: m/z=366.1 [M+H]⁺.

### Step 7: 3-(3-(4-(Chloromethyl)phenyl)-5-(fluoromethoxy)-3H-imidazo[4,5-b]pyridin-2-yl)pyridin-2-amine

To a solution of (4-(2-(2-aminopyridin-3-yl)-5-(fluoromethoxy)-3H-imidazo[4,5-b]pyridin-3-yl)phenyl)methanol (260 mg, 711 μmol) in CH₂Cl₂ (5 mL) was added SOCl₂ (338 mg, 2.85 mmol). The mixture was stirred at 40° C. for 0.5 hr. The mixture was concentrated under reduced pressure, 3-(3-(4-(chloromethyl)phenyl)-5-(fluoromethoxy)-3H-imidazo[4,5-b]pyridin-2-yl)pyridin-2-amine (Intermediate 141, 290 mg, yield: 89%, HCl salt) was obtained as green solid. MS: m/z=384.1 [M+H]⁺.

### Intermediate 142: 3-(3-(4-(Chloromethyl)phenyl)-5-(tetrahydrofuran-3-yl)-3H-imidazo[4,5-b]pyridin-2-yl)pyridin-2-amine

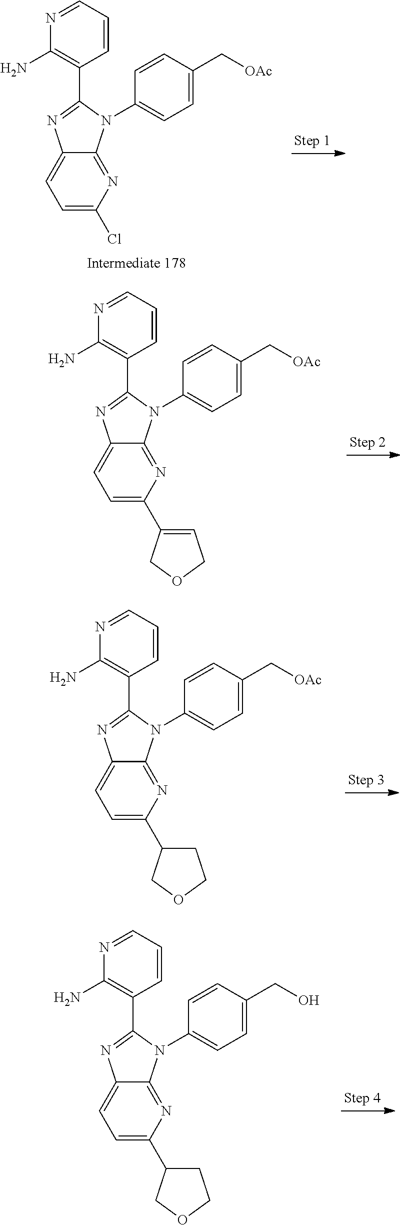 [SMILES: CC(=O)OCc1ccc(-n2c(-c3cccnc3N)nc3ccc(Cl)nc32)cc1 ; CC(=O)OCc1ccc(-n2c(-c3cccnc3N)nc3ccc(C4=CCOC4)nc32)cc1 ; CC(=O)OCc1ccc(-n2c(-c3cccnc3N)nc3ccc(C4CCOC4)nc32)cc1 ; Nc1ncccc1-c1nc2ccc(C3CCOC3)nc2n1-c1ccc(CO)cc1]

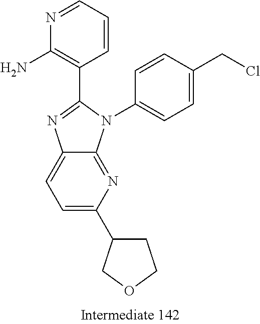 [SMILES: Nc1ncccc1-c1nc2ccc(C3CCOC3)nc2n1-c1ccc(CCl)cc1]

### Step 1: 4-(2-(2-Aminopyridin-3-yl)-5-(2,5-dihydrofuran-3-yl)-3H-imidazo[4,5-b]pyridin-3-yl)benzyl acetate

To a solution of Intermediate 178 (1 g, 2.54 mmol) and 2-(2,5-dihydrofuran-3-yl)-4,4,5,5-tetramethyl-1,3,2-dioxaborolane (547 mg, 2.79 mmol) in DMF (15 mL) were added cataCXiumAPdG3 (71.2 mg, 253 μmol), PCy₃ (185 mg, 254 μmol) and K₃PO₄ (1.08 g, 5.08 mmol). The mixture was stirred at 120° C. for 12 hr. The reaction mixture was diluted with H₂O (30 mL) and extracted with EtOAc (2×40 mL). The combined organic layers were washed with brine (10 mL), dried over Na₂SO₄, filtered and concentrated under reduced pressure. After purified by silica gel flash chromatography (Eluent of 0˜70% EtOAc in petroleum ether), 4-(2-(2-aminopyridin-3-yl)-5-(2,5-dihydrofuran-3-yl)-3H-imidazo[4,5-b]pyridin-3-yl)benzyl acetate (900 mg, yield: 77%) was obtained as a yellow solid. MS: m/z=428.1 [M+H]⁺. ¹H NMR (400 MHz, Chloroform-d) δ 8.07 (dd, J=4.8, 1.6 Hz, 1H), 8.03 (d, J=8.4 Hz, 1H), 7.48 (d, J=8.4 Hz, 2H), 7.43-7.37 (m, 3H), 7.09 (dd, J=8.0 1.6 Hz, 1H), 6.60 (br s, 2H), 6.53 (t, 1=2.0 Hz, 1H), 6.40 (dd, J=8.0, 4.8 Hz, 1H), 5.21 (s, 2H), 5.07-5.03 (m, 2H), 4.90-4.86 (m, 2H), 2.17 (s, 3H).

### Step 2: 4-(2-(2-Aminopyridin-3-yl)-5-(tetrahydrofuran-3-yl)-3H-imidazo[4,5-b]pyridin-3-yl)benzyl acetate

To a solution of 4-(2-(2-aminopyridin-3-yl)-5-(2,5-dihydrofuran-3-yl)-3H-imidazo[4,5-b]pyridin-3-yl)benzyl acetate (550 mg, 1.29 mmol) in MeOH (10 mL) was added Pd/C (68.4 mg, 64.3 μmol, 10% purity). The mixture was degassed and purged with H₂ three times. The mixture was stirred at 40° C. for 16 hr under H₂. The reaction mixture was filtered and concentrated under reduced pressure to give 4-(2-(2-aminopyridin-3-yl)-5-(tetrahydrofuran-3-yl)-3H-imidazo[4,5-b]pyridin-3-yl)benzyl acetate (240 mg, yield: 35%) as a yellow solid. MS: m/z=430.1 [M+H]⁺.

### Step 3: (4-(2-(2-Aminopyridin-3-yl)-5-(tetrahydrofuran-3-yl)-3H-imidazo[4,5-b]pyridin-3-yl)phenyl)methanol

To a solution of 4-(2-(2-aminopyridin-3-yl)-5-(tetrahydrofuran-3-yl)-3H-imidazo[4,5-b]pyridin-3-yl)benzyl acetate (240 mg, 558 μmol) in THF (7 mL) and MeOH (7 mL) was added a solution of K₂CO₃ (77.2 mg, 558 μmol) in H₂O (3.5 mL). The mixture was stirred at 25° C. for 0.5 hr. The reaction mixture was diluted with H₂O (20 mL) and extracted with EtOAc (3×30 mL). The combined organic layers were washed with brine (30 mL), dried over Na₂SO₄, filtered and concentrated under reduced pressure, (4-(2-(2-aminopyridin-3-yl)-5-(tetrahydrofuran-3-yl)-3H-imidazo[4,5-b]pyridin-3-yl)phenyl)methanol (220 mg, crude) was obtained as a yellow solid. MS: m/z=388.0 [M+H]⁺.

### Step 4: 3-(3-(4-(Chloromethyl)phenyl)-5-(tetrahydrofuran-3-yl)-3H-imidazo[4,5-b]pyridin-2-yl)pyridin-2-amine

To a solution of (4-(2-(2-aminopyridin-3-yl)-5-(tetrahydrofuran-3-yl)-3H-imidazo[4,5-b]pyridin-3-yl)phenyl)methanol (220 mg, 567 μmol) in CH₂Cl₂ (10 mL) was added SOCl₂ (405 mg, 3.41 mmol). The mixture was stirred at 40° C. for 0.5 hr. The mixture was concentrated under reduced pressure to give 3-(3-(4-(chloromethyl)phenyl)-5-(tetrahydrofuran-3-yl)-3H-imidazo[4,5-b]pyridin-2-yl)pyridin-2-amine (Intermediate 142, 230 mg, crude) as a yellow solid. MS: m/z=406.0 [M+H]⁺.

### Intermediate 143: 6-(2-(2-Aminopyridin-3-yl)-3-(4-(chloromethyl)phenyl)-3H-imidazo[4,5-b]pyridin-5-yl)nicotinonitrile

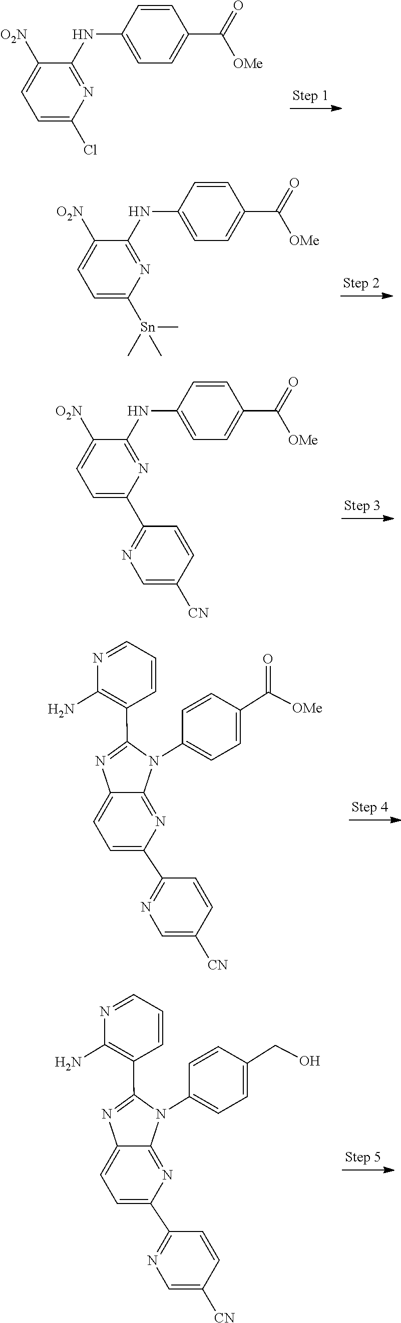

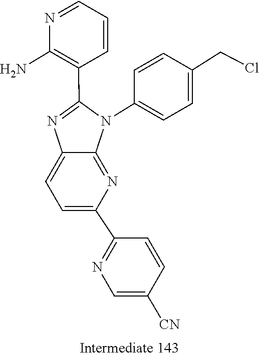 [SMILES: N#Cc1ccc(-c2ccc3nc(-c4cccnc4N)n(-c4ccc(CCl)cc4)c3n2)nc1]

### Step 1: Methyl 4-((3-nitro-6-(trimethylstannyl)pyridin-2-yl)amino)benzoate

A mixture of 4-((6-chloro-3-nitropyridin-2-yl)amino)benzoate (refer to Intermediate 13 for detail procedures, 2 g, 6.5 mmol), Pd(PPh₃)₄ (751 mg, 650 μmol), and 1,1,1,2,2,2-hexamethyldistannane (5.06 g, 15.4 mmol) in 1,4-dioxane (20 mL) was degassed and purged with N₂ three times, and then the mixture was stirred at 125° C. for 2 hr under N₂ atmosphere. The reaction mixture was used for next step directly without work-up and purification. Methyl 4-((3-nitro-6-(trimethylstannyl)pyridin-2-yl)amino)benzoate (2.83 g) was obtained as a black oil. MS: m/z=438.1 [M+H]⁺.

### Step 2: Methyl 4-((5-amino-5′-cyano-[2,2′-bipyridin]-6-yl)amino)benzoate

A mixture of methyl 4-((3-nitro-6-(trimethylstannyl)pyridin-2-yl)amino)benzoate (2.83 g, 6.49 mmol), 6-bromonicotinonitrile (594 mg, 3.25 mmol), and Pd(PPh₃)₄ (375 mg, 325 μmol) in 1,4-dioxane (30 mL) was degassed and purged with N₂ three times, then the mixture was stirred at 100° C. for 2 hr under N₂ atmosphere. The reaction was concentrated under reduced pressure. After purified by silica gel flash chromatography (Eluent of 1˜33%, EtOAc in petroleum ether), methyl 4-((5-amino-5′-cyano-[2,2′-bipyridin]-6-yl)amino)benzoate (380 mg, yield: 17% for two steps) was obtained as a yellow solid. MS: m/z=346.3 [M+H]⁺. ¹H NMR (400 MHz, Dimethylsulfoxide-d₆) 8.95 (d, J=1.2 Hz, 1H), 8.51 (s, 1H), 8.29 (dd, J=8.4, 2.0 Hz, 1H), 8.22-8.19 (m, 1H), 7.95-7.89 (m, 3H), 7.82-7.79 (m, 2H), 7.08 (d, J=8.4 Hz, 1H), 3.82 (s, 3H).

### Step 3: Methyl 4-(2-(2-aminopyridin-3-yl)-5-(5-cyanopyridin-2-yl)-3H-imidazo[4,5-b]pyridin-3-yl)benzoate

To a solution of methyl 4-((5-amino-5′-cyano-[2,2′-bipyridin]-6-yl)amino)benzoate (160 mg, 426 μmol) in DMSO (5 mL) were added Na₂S₂O₄ (349 mg, 1.71 mmol 85% purity) and 2-aminonicotinaldehyde (62.5 mg, 512 μmol). The mixture was stirred at 100° C. for 16 hr. The reaction mixture was quenched with H₂O (10 mL) at 25° C., and then diluted with water (30 mL) and extracted with CH₂Cl₂ (50 mL×3). The combined organic layers were washed with brine (50 mL), dried over Na₂SO₄, filtered and concentrated under reduced pressure. After purified by silica gel flash chromatography (Eluent of 1˜50% EtOAc in petroleum ether), methyl 4-(2-(2-aminopyridin-3-yl)-5-(5-cyanopyridin-2-yl)-3H-imidazo[4,5-b]pyridin-3-yl)benzoate (100 mg, yield: 47%) was obtained as a yellow solid. MS: m/z=448.2 [M+H]⁺. ¹H NMR (400 MHz, Chloroform-d) δ 8.93 (s, 1H), 8.73-8.65 (m, 1H), 8.40 (d, J=7.6 Hz, 1H), 8.34-8.28 (m, 3H), 8.27-8.09 (m, 2H), 8.00-7.96 (m, 2H), 7.58 (d, J=7.2 Hz, 2H), 7.32 (d, J=7.6 Hz, 1H), 6.56-6.46 (m, 1H), 4.03 (s, 3H).

### Step 4: 6-(2-(2-Aminopyridin-3-yl)-3-(4-(hydroxymethyl)phenyl)-3H-imidazo[4,5-b]pyridin-5-yl)nicotinonitrile

To a solution of methyl 4-(2-(2-aminopyridin-3-yl)-5-(5-cyanopyridin-2-yl)-3H-imidazo[4,5-b]pyridin-3-yl)benzoate (100 mg, 223 μmol) in THF (2 mL) was added LiAlH₄ (12.7 mg, 335 μmol). The mixture was stirred at 0° C. for 1 hr. The reaction mixture was quenched with Na₂SO₄·10H₂O (100 mg) at 0° C., filtered and concentrated under reduced pressure to give 6-(2-(2-aminopyridin-3-yl)-3-(4-(hydroxymethyl)phenyl)-3H-imidazo[4,5-b]pyridin-5-yl)nicotinonitrile (60 mg, yield: 15%) as a yellow solid. MS: m/z=420.2 [M+H]⁺.

### Step 5: 6-(2-(2-Aminopyridin-3-yl)-3-(4-(chloromethyl)phenyl)-3H-imidazo[4,5-b]pyridin-5-yl)nicotinonitrile

To a solution of 6-(2-(2-aminopyridin-3-yl)-3-(4-(hydroxymethyl)phenyl)-3H-imidazo[4,5-b]pyridin-5-yl)nicotinonitrile (60 mg, 143 μmol) in CH₂Cl₂ (5 mL) was added SOCl₂ (34 mg, 286 μmol) at 0° C. Then the mixture was stirred at 40° C. for 1 hr. The reaction mixture was concentrated under reduced pressure to give 6-(2-(2-aminopyridin-3-yl)-3-(4-(chloromethyl)phenyl)-3H-imidazo[4,5-b]pyridin-5-yl)nicotinonitrile (Intermediate 143, 70 mg, yield: 45%, HCl salt) as a yellow solid. MS: m/z=438.2 [M+H]⁺.

### Intermediate 144: 5-(2-(2-Aminopyridin-3-yl)-3-(4-(chloromethyl)phenyl)-3H-imidazo[4,5-b]pyridin-5-yl)picolinonitrile

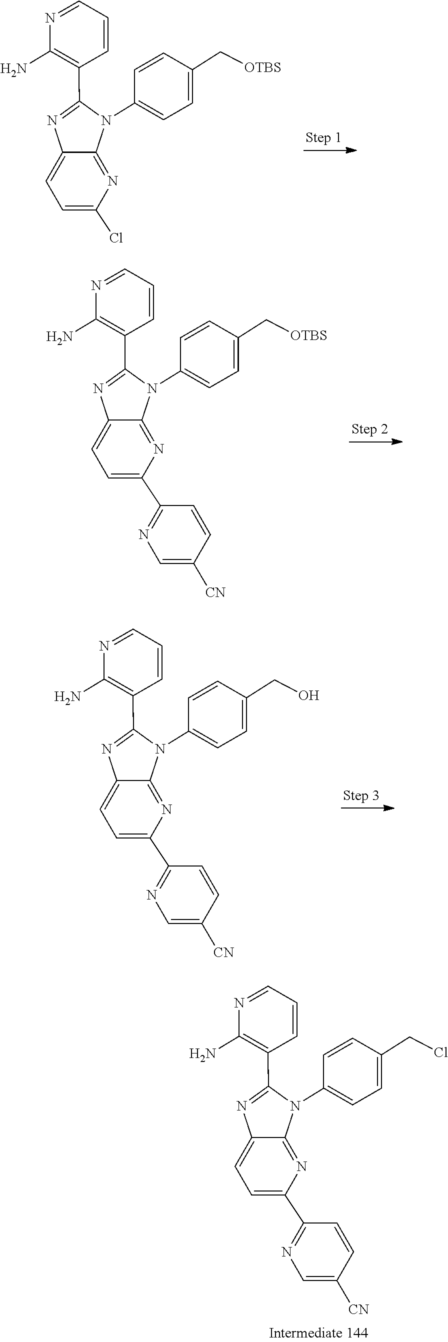

### Step 1: 5-(2-(2-Aminopyridin-3-yl)-3-(4-(((tert-butyldimethylsilyl)oxy)methyl)phenyl)-3H-imidazo[4,5-b]pyridin-5-yl)picolinonitrile

A mixture of 3-(3-(4-(((tert-butyldimethylsilyl)oxy)methyl)phenyl)-5-chloro-3H-imidazo[4,5-b]pyridin-2-yl)pyridin-2-amine (refer to Intermediate 117 for detail procedures, 700 mg, 1.50 mmol), (6-cyanopyridin-3-yl)boronic acid (267 mg, 1.80 mmol), Cs₂CO₃ (1.47 g, 4.51 mmol), and Pd(dppf)Cl₂ (220 mg, 300 μmol) in 1,4-dioxane (10 mL) and H₂O (2 mL) was degassed and purged with N₂ three times. The mixture was stirred at 80° C. for 2 hr under N₂ atmosphere. The reaction mixture was diluted with CH₂Cl₂ (50 mL) and extracted with H₂O (50 mL×3). The combined organic layers were washed with brine (50 mL), dried over Na₂SO₄, filtered and concentrated under reduced pressure. After purified by silica gel flash chromatography (Eluent of 50˜100% EtOAc in petroleum ether), 5-(2-(2-aminopyridin-3-yl)-3-(4-(((tert-butyldimethylsilyl)oxy)methyl)phenyl)-3H-imidazo[4,5-b]pyridin-5-yl)picolinonitrile (700 mg, yield: 74%) was obtained as a yellow solid. MS: m/z=534.1 [M+H]⁺.

### Step 2: 5-(2-(2-Aminopyridin-3-yl)-3-(4-(hydroxymethyl)phenyl)-3H-imidazo[4,5-b]pyridin-5-yl)picolinonitrile

To a solution of 5-(2-(2-aminopyridin-3-yl)-3-(4-(((tert-butyldimethylsilyl)oxy)methyl)phenyl)-3H-imidazo[4,5-b]pyridin-5-yl)picolinonitrile (100 mg, 187 μmol) in THF (5 mL) was added TBAF (73.5 mg, 281 μmol, 1 M). The mixture was stirred at 25° C. for 0.5 hr. The reaction mixture was quenched with H₂O (20 mL) at 25° C. and extracted with CH₂Cl₂ (50 mL×3). The combined organic layers were washed with brine (50 mL), dried over Na₂SO₄, filtered and concentrated under reduced pressure to give 5-(2-(2-aminopyridin-3-yl)-3-(4-(hydroxymethyl)phenyl)-3H-imidazo[4,5-b]pyridin-5-yl)picolinonitrile (100 mg, yield: 96%) as a yellow solid. MS: m/z=420.0 [M+H]⁺.

### Step 3: 5-(2-(2-Aminopyridin-3-yl)-3-(4-(chloromethyl)phenyl)-3H-imidazo[4,5-b]pyridin-5-yl)picolinonitrile

To a solution of 5-(2-(2-aminopyridin-3-yl)-3-(4-(hydroxymethyl)phenyl)-3H-imidazo[4,5-b]pyridin-5-yl)picolinonitrile (100 mg, 238 μmol) in CH₂Cl₂ (3 mL) was added SOCl₂ (56.7 mg, 477 μmol) at 0° C. Then the mixture was stirred at 40° C. for 1 hr. The reaction mixture was quenched with H₂O (1 mL) at 25° C., and concentrated under reduced pressure to give 5-(2-(2-aminopyridin-3-yl)-3-(4-(chloromethyl)phenyl)-3H-imidazo[4,5-b]pyridin-5-yl)picolinonitrile (Intermediate 144, 120 mg, HCl salt, yield: 82%) as a yellow solid. MS: m/z=438.2 [M+H]⁺.

### Intermediate 145: 4-(2-(2-Aminopyridin-3-yl)-5-(oxetan-3-yl)-3H-imidazo[4,5-b]pyridin-3-yl)benzyl methanesulfonate

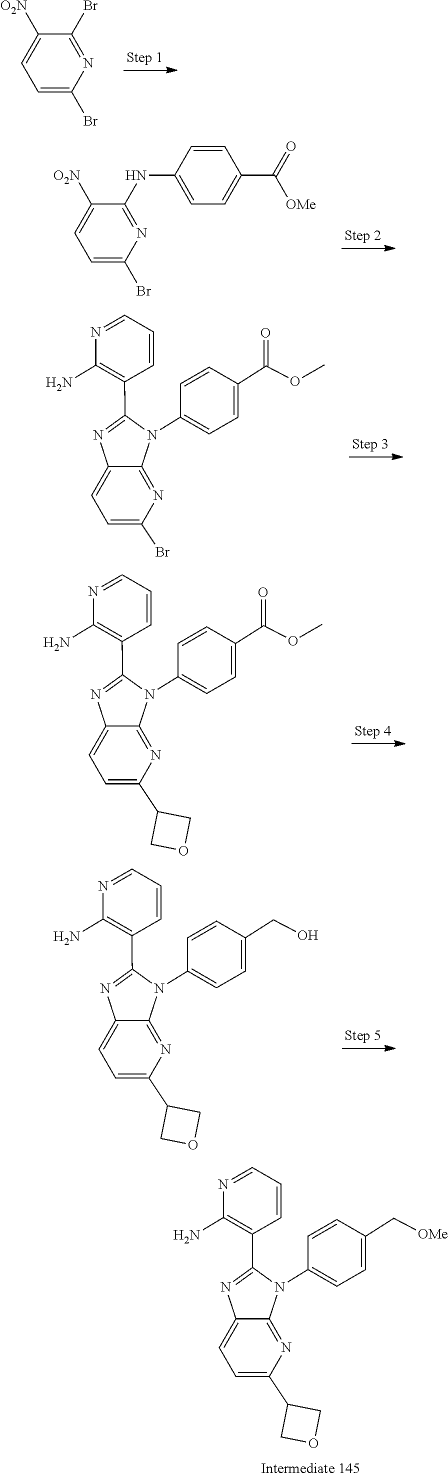

### Step 1: Methyl 4-((6-bromo-3-nitropyridin-2-yl)amino)benzoate

To a solution of 2,6-dibromo-3-nitropyridine (5 g, 17.7 mmol) and methyl 4-aminobenzoate (2.68 g, 17.7 mmol) in 1,4-dioxane (50 mL) was added DIEA (4.58 g, 35.5 mmol). The mixture was stirred at 50° C. for 12 hr under N₂. The mixture was concentrated under reduced pressure. The crude was triturated with EtOAc (100 mL) at 25° C. for 30 min to give methyl 4-((6-bromo-3-nitropyridin-2-yl)amino)benzoate (4.4 g, yield: 70%) as a red solid. MS: m/z=351.9, 353.9 [M+H]⁺.

### Step 2: Methyl 4-(2-(2-aminopyridin-3-yl)-5-bromo-3H-imidazo[4,5-b]pyridin-3-yl)benzoate

To a solution of methyl 4-((6-bromo-3-nitropyridin-2-yl)amino)benzoate (2 g, 5.68 mmol) and 2-aminopyridine-3-carbaldehyde (763 mg, 6.25 mmol) in DMSO (20 mL) was added Na₂S₂O₄ (2.47 g, 14.2 mmol). The mixture was stirred at 100° C. for 12 hr under N₂. Sat. aq. NaHCO₃ (50 mL) was added and the mixture was extracted with CH₂Cl₂/MeOH (10/1, 50 mL×3). The combined organic layers were washed with brine (50 mL×3), dried over Na₂SO₄, filtered and concentrated. After purified by silica gel flash chromatography (Eluent of 0˜100% EtOAc in petroleum ether), methyl 4-(2-(2-aminopyridin-3-yl)-5-bromo-3H-imidazo[4,5-b]pyridin-3-yl)benzoate (1.3 g, yield: 54%) was obtained as a black brown solid. MS: m/z=423.9, 425.9 [M+H]⁺. ¹H NMR (400 MHz, Chloroform-d) δ 8.25-8.15 (m, 2H), 8.10-8.00 (m, 1H), 7.95 (d, J=8.4 Hz, 1H), 7.55-7.40 (m, 3H), 7.05-7.00 (m, 1H), 6.83 (br s, 2H), 6.45-6.30 (m, 1H), 3.97 (s, 3H).

### Step 3: Methyl 4-(2-(2-aminopyridin-3-yl)-5-(oxetan-3-yl)-3H-imidazo[4,5-b]pyridin-3-yl)benzoate

To a solution of methyl 4-(2-(2-aminopyridin-3-yl)-5-bromo-3H-imidazo[4,5-b]pyridin-3-yl)benzoate (500 mg, 1.18 mmol) and 3-bromooxetane (242 mg, 1.77 mmol) in DME (8 mL) were added bis(trimethylsilyl)silyl-trimethyl-silane (293 mg, 1.18 mmol), Na₂CO₃ (250 mg, 2.36 mmol), 4-tert-butyl-2-(4-tert-butyl-2-pyridyl)pyridine (15.8 mg, 59.0 μmol), (Ir[df(CF₃)ppy]₂(dtbpy))PF₆ (13.2 mg, 11.8 μmol), and NiCl₂.glyme (12.9 mg, 58.9 μmol). The mixture was stirred at 25° C. for 16 hr under 450 nm blue-light with fan cooling at 5000 rpm. H₂O (50 mL) was added and the mixture was extracted with CH₂Cl₂/MeOH (10/1, 30 mL×3). The combined organic layers were washed with brine (50 mL), dried over Na₂SO₄, filtered and concentrated. After purified by silica gel flash chromatography (Eluent of 0˜100% EtOAc in petroleum ether), methyl 4-(2-(2-aminopyridin-3-yl)-5-(oxetan-3-yl)-3H-imidazo[4,5-b]pyridin-3-yl)benzoate (170 mg, yield: 36%) was obtained as a black brown oil. MS: m/z=402.2 [M+H]⁺.

### Step 4: (4-(2-(2-Aminopyridin-3-yl)-5-(oxetan-3-yl)-3H-imidazo[4,5-b]pyridin-3-yl)phenyl)methanol

To a solution of methyl 4-(2-(2-aminopyridin-3-yl)-5-bromo-3H-imidazo[4,5-b]pyridin-3-yl)benzoate (170 mg, 424 μmol) in THF (4 mL) was added LiAlH₄ (32.2 mg, 847 μmol) in portions under N₂ at 0° C. The mixture was stirred at 0° C. for 1 hr under N₂. The mixture was diluted with THF (20 mL). Na₂SO₄ 10H₂O was added in portions until no bubbles were formed. The resulting mixture was stirred at 25° C. for 20 min and filtered. The filter cake was washed with THF (20 mL×2), the combined filtrate was concentrated under reduced pressure. After purified by silica gel flash chromatography (Eluent of 0˜100% EtOAc in petroleum ether), (4-(2-(2-aminopyridin-3-yl)-5-(oxetan-3-yl)-3H-imidazo[4,5-b]pyridin-3-yl)phenyl)methanol (50 mg, yield: 32%) was obtained as yellow oil. MS: m/z=374.2 [M+H]⁺.

### Step 5: 4-(2-(2-Aminopyridin-3-yl)-5-(oxetan-3-yl)-3H-imidazo[4,5-b]pyridin-3-yl)benzyl methanesulfonate

To a solution of (4-(2-(2-aminopyridin-3-yl)-5-(oxetan-3-yl)-3H-imidazo[4,5-b]pyridin-3-yl)phenyl)methanol (50 mg, 134 μmol) in CH₂Cl₂ (1 mL) were added TEA (40.7 mg, 402 μmol) and MsCl (100 mg, 873 μmol) at 0° C. The mixture was stirred at 0° C. for 2 hr under N₂. The mixture was added dropwise into H₂O (20 ml) at 0° C. The mixture was extracted with CH₂Cl₂ (10 mL×2). The combined organic layers were dried over Na₂SO₄, filtered and concentrated. 4-(2-(2-Aminopyridin-3-yl)-5-(oxetan-3-yl)-3H-imidazo[4,5-b]pyridin-3-yl)benzyl methanesulfonate (Intermediate 145, 60.5 mg) was obtained as yellow oil, which was used in the next step directly. MS: m/z=452.1 [M+H]⁺.

### Intermediate 146: 3-(3-(4-(Chloromethyl)phenyl)-5-(4-methoxyphenyl)-3H-imidazo[4,5-b]pyridin-2-yl)pyridin-2-amine

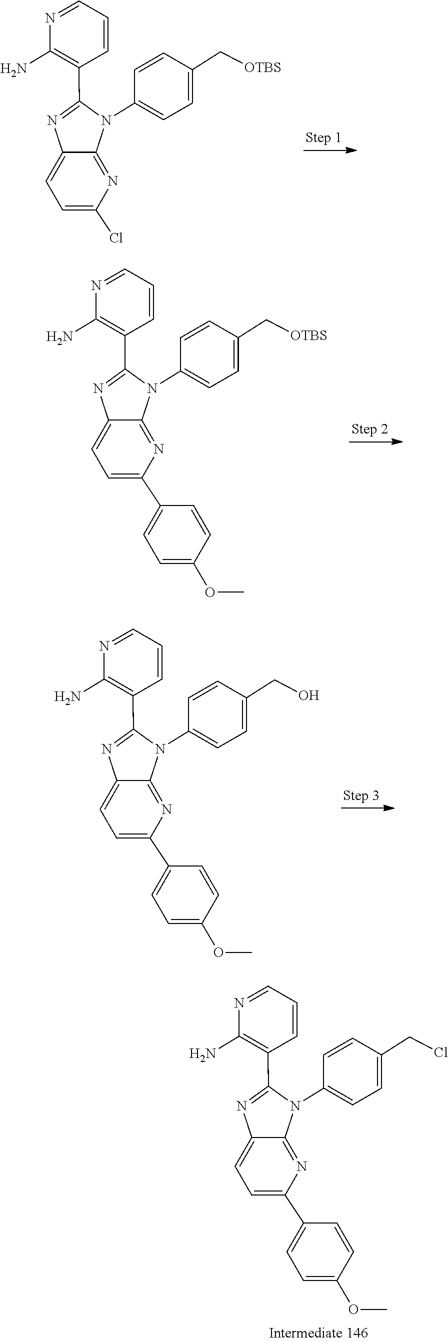

### Step 1: 3-(3-(4-(((Tert-butyldimethylsilyl)oxy)methyl)phenyl)-5-(4-methoxyphenyl)-3H-imidazo[4,5-b]pyridin-2-yl)pyridin-2-amine

To a solution of 3-(3-(4-(((tert-butyldimethylsilyl)oxy)methyl)phenyl)-5-chloro-3H-imidazo[4,5-b]pyridin-2-yl)pyridin-2-amine (refer to Intermediate 117 for detail procedures, 350 mg, 751 μmol) and (4-methoxyphenyl)boronic acid (126 mg, 826 μmol) in 1,4-dioxane (4 mL) and H₂O (0.8 mL) were added Pd(dppf)Cl₂ (55.0 mg, 75.1 μmol) and Cs₂CO₃ (734 mg, 2.25 mmol). The mixture was degassed and purged with N₂ three times and then was stirred at 100° C. for 2 hr under N₂. H₂O (50 mL) was added, and the mixture was extracted with EtOAc (30 mL×3). The combined organic layers were washed with brine (50 mL), dried over Na₂SO₄, filtered and concentrated. After purified by silica gel flash chromatography (Eluent of 0˜70% EtOAc in petroleum ether), 3-(3-(4-(((tert-butyldimethylsilyl)oxy)methyl)phenyl)-5-(4-methoxyphenyl)-3H-imidazo[4,5-b]pyridin-2-yl)pyridin-2-amine (330 mg, yield: 82%) was obtained as a brown solid. MS: m/z=538.3 [M+H]⁺.

### Step 2: (4-(2-(2-Aminopyridin-3-yl)-5-(4-methoxyphenyl)-3H-imidazo[4,5-b]pyridin-3-yl)phenyl)methanol

To a solution of 3-(3-(4-(((tert-butyldimethylsilyl)oxy)methyl)phenyl)-5-(4-methoxyphenyl)-3H-imidazo[4,5-b]pyridin-2-yl)pyridin-2-amine (330 mg, 614 μmol) in THF (8 mL) was added TBAF (1 M). The mixture was stirred at 25° C. for 2 hr under N₂. H₂O (30 mL) was added and the mixture was extracted with EtOAc (10 mL×2). The combined organic layers were dried over anhydrous Na₂SO₄, filtered and concentrated. (4-(2-(2-Aminopyridin-3-yl)-5-(4-methoxyphenyl)-3H-imidazo[4,5-b]pyridin-3-yl)phenyl)methanol (250 mg, yield: 96%) was obtained as a black brown solid, which was used in the next step directly. MS: m/z=424.2 [M+H]⁺.

### Step 3: 3-(3-(4-(Chloromethyl)phenyl)-5-(4-methoxyphenyl)-3H-imidazo[4,5-b]pyridin-2-yl)pyridin-2-amine

To a solution of (4-(2-(2-aminopyridin-3-yl)-5-(4-methoxyphenyl)-3H-imidazo[4,5-b]pyridin-3-yl)phenyl)methanol (250 mg, 590 μmol) in CH₂Cl₂ (3 mL) was added SOCl₂ (211 mg, 1.77 mmol). The mixture was stirred at 25° C. for 2 hr under N₂. The mixture was concentrated to give 3-(3-(4-(Chloromethyl)phenyl)-5-(4-methoxyphenyl)-3H-imidazo[4,5-b]pyridin-2-yl)pyridin-2-amine (Intermediate 146, 200 mg, yield: 77%) as a black brown solid, which was used in the next step directly. MS: m/z=442.3 [M+H]⁺.

### Intermediate 147: 3-(3-(4-(Chloromethyl)phenyl)-5-(2-methoxyphenyl)-3H-imidazo[4,5-b]pyridin-2-yl)pyridin-2-amine

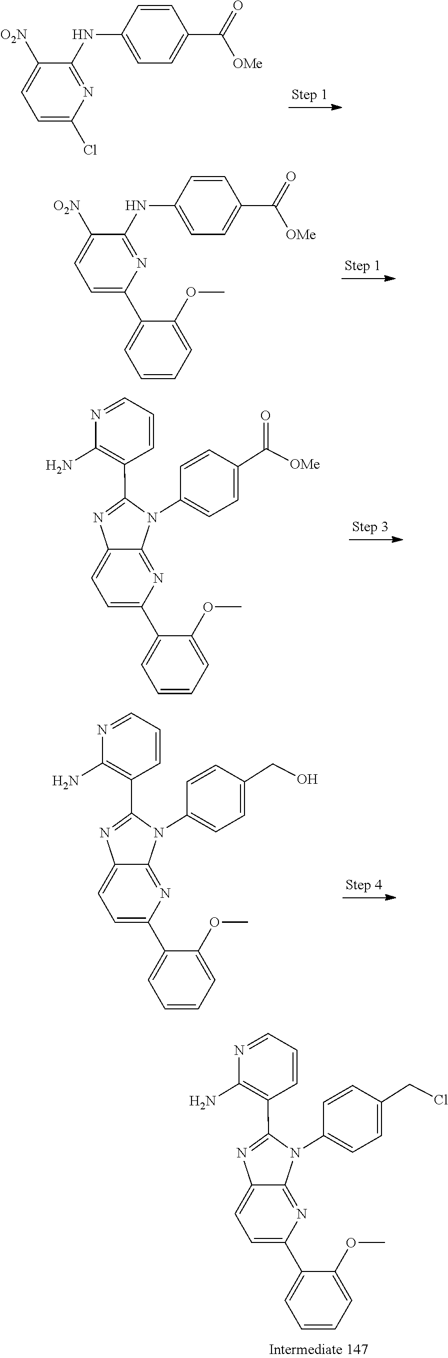

### Step 1: Methyl 4-((6-(2-methoxyphenyl)-3-nitropyridin-2-yl)amino)benzoate

A mixture of methyl 4-((6-chloro-3-nitropyridin-2-yl)amino)benzoate (refer to Intermediate 13 for detail procedures, 5 g, 16.3 mmol), (2-methoxyphenyl)boronic acid (2.47 g, 16.3 mmol), Cs₂CO₃ (15.9 g, 48.8 mmol), and Pd(dppf)Cl₂ (595 mg, 813 μmol) in 1,4-dioxane (100 mL) and H₂O (20 mL) was degassed and purged with N₂ three times. The mixture was stirred at 80° C. for 16 hr under N₂ atmosphere. The reaction mixture was diluted with H₂O (100 mL) and extracted with EtOAc (50 mL×3). The combined organic layers were washed with brine (30 mL×3), dried over Na₂SO₄, filtered and concentrated under reduced pressure. After purified by silica gel flash chromatography (Eluent of 15˜20% EtOAc in CH₂Cl₂), methyl 4-((6-(2-methoxyphenyl)-3-nitropyridin-2-yl)amino)benzoate (4 g, yield: 59%) was obtained as an orange red solid. MS: m/z=380.1 [M+H]⁺.

### Step 2: Methyl 4-(2-(2-aminopyridin-3-yl)-5-(2-methoxyphenyl)-3H-imidazo[4,5-b]pyridin-3-yl)benzoate

To a solution of methyl 4-((6-(2-methoxyphenyl)-3-nitropyridin-2-yl)amino)benzoate (4 g, 10.5 mmol), 2-aminonicotinaldehyde (1.55 g, 12.7 mmol) in DMSO (100 mL) was added Na₂S₂O₄ (7.34 g, 42.2 mmol). The mixture was stirred at 100° C. for 16 hr. The reaction mixture was diluted with water (100 mL) and extracted with CH₂Cl₂ (100 mL×3). The combined organic layers were washed with brine (250 mL×2), dried over Na₂SO₄, filtered and concentrated under reduced pressure. After purified by silica gel flash chromatography (Eluent of 25˜30% EtOAc in CH₂Cl₂), methyl 4-(2-(2-aminopyridin-3-yl)-5-(2-methoxyphenyl)-3H-imidazo[4,5-h]pyridin-3-yl)benzoate (3 g, yield: 59%) was obtained as an orange red solid. MS: m/z=452.5 [M+H]⁺. ¹H NMR (400 MHz, Chloroform-d) δ 8.19 (d, J=8.4 Hz, 2H), 8.11 (d, J=8.4 Hz, 1H), 8.08 (dd, J=4.8, 1.6 Hz, 1H), 7.98 (d, J=8.4 Hz, 1H), 7.78 (dd, J=7.6, 1.6 Hz, 1H), 7.55 (d, J=8.8 Hz, 2H), 7.39-7.33 (m, 1H), 7.09-7.00 (m, 3H), 6.67 (br s, 2H), 6.41 (dd, J=7.6, 4.8 Hz, 1H), 3.97 (s, 3H), 3.89 (s, 3H).

### Step 3: (4-(2-(2-Aminopyridin-3-yl)-5-(2-methoxyphenyl)-3H-imidazo[4,5-b]pyridin-3-yl)phenyl)methanol

To a solution of methyl 4-(2-(2-aminopyridin-3-yl)-5-(2-methoxyphenyl)-3H-imidazo[4,5-b]pyridin-3-yl)benzoate (500 mg, 1.11 mmol) in THF (10 mL) was added LiAlH₄ (2.5 M, 886 μL) at 0° C. The mixture was stirred at 25° C. for 2 hr. The reaction mixture was quenched with Na₂SO₄-10H₂O (1 g) at 0° C. The mixture was filtered, and the filter cake was washed with CH₂Cl₂ (10 mL×3). The filtrate was concentrated under reduced pressure to give (4-(2-(2-aminopyridin-3-yl)-5-(2-methoxyphenyl)-3H-imidazo[4,5-b]pyridin-3-yl)phenyl)methanol (460 mg, yield: 73%) as a yellow solid, which was used to the next step without purification. MS: m/z=424.0 [M+H]⁺.

### Step 4: 3-(3-(4-(Chloromethyl)phenyl)-5-(2-methoxyphenyl)-3H-imidazo[4,5-b]pyridin-2-yl)pyridin-2-amine

To a solution of (4-(2-(2-aminopyridin-3-yl)-5-(2-methoxyphenyl)-3H-imidazo[4,5-b]pyridin-3-yl)phenyl)methanol (460 mg, 1.09 mmol) in CH₂Cl₂ (5 mL) was added SOCl₂ (388 mg, 3.26 mmol) at 0° C. The mixture was stirred at 40° C. for 1 hr. The reaction mixture was quenched with H₂O (0.5 mL) at 0° C., then filtered and concentrated under reduced pressure to give 3-(3-(4-(chloromethyl)phenyl)-5-(2-methoxyphenyl)-3H-imidazo[4,5-b]pyridin-2-yl)pyridin-2-amine (Intermediate 147, 500 mg, yield: 96%) as a black brown solid. MS: m/z=442.0 [M+H]^{+ 1}H NMR (400 MHz, Methanol-d₄) δ 8.31 (d, J=8.8 Hz, 1H), 8.09 (d, J=4.8 Hz, 1H), 8.05 (dd, J=6.4, 1.2 Hz, 1H), 7.95 (dd, J=7.6, 1.2 Hz, 1H), 7.71-7.65 (m, 3H), 7.60-7.56 (m, 2H), 7.43-7.38 (m, 1H), 7.13 (d, J=8.0 Hz, 1H), 7.04-6.99 (m, 1H), 6.91-6.88 (m, 1H), 4.76 (s, 2H), 3.88 (s, 3H).

### Intermediate 148: 3-(3-(4-(Chloromethyl)phenyl)-5-(trifluoromethoxy)-3H-imidazo[4,5-b]pyridin-2-yl)pyridin-2-amine

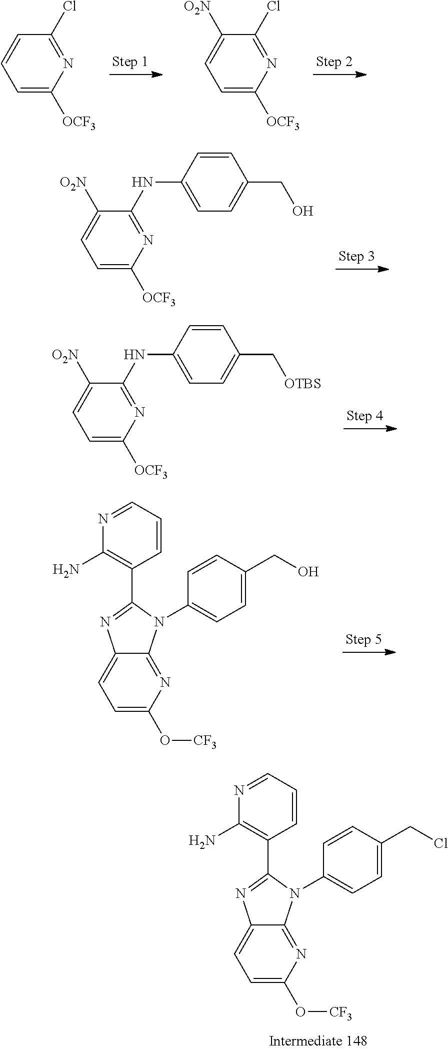

### Step 1; 2-Chloro-3-nitro-6-(trifluoromethoxy)pyridine

To a solution of 2-chloro-6-(trifluoromethoxy)pyridine (2.0 g, 10.1 mmol) in H₂SO₄ (25 mL) at 0° C. was added HNO₃ (18.9 g, 300 mmol) dropwise and stirred for 1 hr. This mixture was stirred at 40° C. for 12 hr. This mixture was poured into ice water (30 mL) and extracted with CH₂Cl₂ (50 mL×3). The combined organic layers were washed with brine (50 mL×3), dried over Na₂SO₄, filtered and concentrated to give 2-chloro-3-nitro-6-(trifluoromethoxy)pyridinee (2.40 g, yield: 97%) as yellow oil, which was used in the next step without purification.

### Step 2: (4-((3-Nitro-6-(trifluoromethoxy)pyridin-2-yl)amino)phenyl)methanol

To a mixture of 2-chloro-3-nitro-6-(trifluoromethoxy)pyridinee (1.80 g, 7.42 mmol) in THF (25 mL) were added DIEA (2.88 g, 22.3 mmol) and (4-aminophenyl)methanol (1.01 g, 8.16 mmol) at 25° C., the mixture was stirred at 80° C. for 1 hr. The mixture was quenched with water (30 mL) and then extracted with EtOAc (30 mL×3). The combined organic layers were filtered and concentrated to give the crude. After purified by silica gel flash chromatography (Eluent of 0˜30% EtOAc in petroleum ether), (4-((3-nitro-6-(trifluoromethoxy)pyridin-2-yl)amino)phenyl)methanol (900 mg, yield: 36%) was obtained as a yellow solid. ¹H NMR (400 MHz, Dimethylsulfoxide-d₆) δ 10.26 (s, 1H), 8.70 (d, J=8.8 Hz, 1H), 7.54 (d, J=8.4 Hz, 2H), 7.32 (d, J=8.8 Hz, 2H), 6.70 (d, J=8.8 Hz, 1H), 5.18 (t, J=5.6 Hz, 1H), 4.50 (d, J=5.6 Hz, 2H). ¹⁹F NMR (376.5 MHz, Dimethylsulfoxide-d₆) δ −55.544.

### Step 3: N-(4-(((Tert-butyldimethylsilyl)oxy)methyl)phenyl)-3-nitro-6-(trifluoromethoxy)pyridin-2-amine

To a solution of (4-((3-Nitro-6-(trifluoromethoxy)pyridin-2-yl)amino)phenyl)methanol (900 mg, 2.73 mmol) in CH₂Cl₂ (8 mL) were added imidazole (279 mg, 4.10 mmol) and TBSCl (536 mg, 3.55 mmol) at 25° C. This mixture was stirred at 25° C. for 1 hr. The mixture was filtered and concentrated to give N-(4-(((tert-butyldimethylsilyl)oxy)methyl)phenyl)-3-nitro-6-(trifluoromethoxy)pyridin-2-amine (1.20 g, yield: 99%) as a yellow solid, which was used in the next step without further purification. MS: m/z=444.2 [M+H]⁺. ¹H NMR (400 MHz, Dimethylsulfoxide-d₆) δ 10.25 (s, 1H), 8.69 (d, J=8.8 Hz, 1H), 7.53 (d, J=8.4 Hz, 2H), 7.30 (d, J=8.4 Hz, 2H), 6.69 (d, J=8.8 Hz, 1H), 4.71 (s, 2H), 0.89 (s, 9H), 0.07 (s, 6H). ¹⁹F NMR (400 MHz, Dimethylsulfoxide-d₆) δ −55.611.

### Step 4: (4-(2-(2-Aminopyridin-3-yl)-5-(trifluoromethoxy)-3H-imidazo[4,5-b]pyridin-3-yl)phenyl)methanol

To a solution of N-(4-(((tert-butyldimethylsilyl)oxy)methyl)phenyl)-3-nitro-6-(trifluoromethoxy)pyridin-2-amine (1.20 g, 2.71 mmol) in DMSO (10 mL) were added Na₂S₂O₄ (1.18 g, 6.76 mmol) and 2-aminopyridine-3-carbaldehyde (363 mg, 2.98 mmol) at 25° C. The mixture was stirred at 100° C. for 12 hr. NaHCO₃ (sat, 50 mL) was added and the mixture was extracted with CH₂Cl₂ (50 mL×3). The combined organic layers were washed with brine (50 mL×3), dried over Na₂SO₄, filtered and concentrated to give the crude. After purified by silica gel flash chromatography (Eluent of 0˜100% EtOAc/EtOH (3/1) in petroleum ether), (4-(2-(2-aminopyridin-3-yl)-5-(trifluoromethoxy)-3H-imidazo[4,5-b]pyridin-3-yl)phenyl)methanol (400 mg, yield: 37%) was obtained as a brown oil. MS: m/z=402.0 [M+H]+. ¹H NMR (400 MHz, Dimethylsulfoxide-d₆) δ 8.41 (d, J=8.0 Hz, 1H), 7.99 (dd, J=4.8, 1.6 Hz, 1H), 7.45 (d, J=8.4 Hz, 2H), 7.38 (d, J=8.0 Hz, 2H), 7.31-7.21 (m, 2H), 6.79 (s, 2H), 6.43 (dd, J=7.6, 4.8 Hz, 1H), 5.34 (t, J=6.0 Hz, 1H), 4.57 (d, J=5.2 Hz, 2H). ¹⁹F NMR (400 MHz, Dimethylsulfoxide-d₆) δ −55.482.

### Step 5: 3-(3-(4-(Chloromethyl)phenyl)-5-(trifluoromethoxy)-3H-imidazo[4,5-b]pyridin-2-yl)pyridin-2-amine

To a solution of (4-(2-(2-aminopyridin-3-yl)-5-(trifluoromethoxy)-3H-imidazo[4,5-b]pyridin-3-yl)phenyl)methanol (400 mg, 997 μmol) in CH₂C12 (5 mL) was added SOCl₂ (356 mg, 2.99 mmol) at 25° C. This mixture was stirred at 25° C. for 1 hr. The reaction mixture was concentrated to give 3-(3-(4-(chloromethyl)phenyl)-5-(trifluoromethoxy)-3H-imidazo[4,5-b]pyridin-2-yl)pyridin-2-amine (Intermediate 148, 220 mg) as a brown solid, which was used in the next step without purification. MS: m/z=420.1 [M+H]+.

### Intermediate 149: 3-(3-(4-(Chloromethyl)phenyl)-5-isopropoxy-3H-imidazo[4,5-b]pyridin-2-yl)pyridin-2-amine

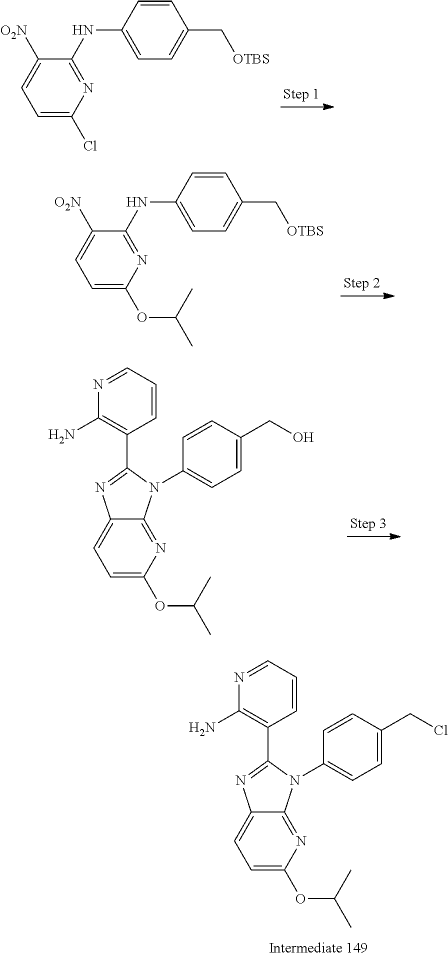 [SMILES: CC(C)(C)[Si](C)(C)OCc1ccc(Nc2nc(Cl)ccc2[N+](=O)[O-])cc1 ; CC(C)Oc1ccc([N+](=O)[O-])c(Nc2ccc(CO[Si](C)(C)C(C)(C)C)cc2)n1 ; CC(C)Oc1ccc2nc(-c3cccnc3N)n(-c3ccc(CO)cc3)c2n1 ; CC(C)Oc1ccc2nc(-c3cccnc3N)n(-c3ccc(CCl)cc3)c2n1]

### Step 1: N-(4-(((tert-butyldimethylsilyl)oxy)methyl)phenyl)-6-isopropoxy-3-nitropyridin-2-amine

To a mixture of N-(4-(((tert-butyldimethylsilyl)oxy)methyl)phenyl)-6-chloro-3-nitropyridin-2-amine (refer to Intermediate 105 for detail procedures, 1 g, 2.54 mmol) in i-PrOH (10 mL) was added NaH (203 mg, 5.08 mmol, 60% purity). The mixture was stirred at 20° C. for 12 hr. H₂O (50 mL) was added, and the mixture was extracted with EtOAc (100 mL×2). The combined organic layers were washed with brine (50 mL), dried over Na₂SO₄, filtered and concentrated. After purified by silica gel flash chromatography (Eluent of 0˜30% EtOAc in petroleum ether), N-(4-(((tert-butyldimethylsilyl)oxy)methyl)phenyl)-6-isopropoxy-3-nitropyridin-2-amine (710 mg, yield: 67%) was obtained as a yellow solid. MS: m/z=418.1 [M+H]⁺. ¹H NMR (400 MHz, Dimethylsulfoxide-d₆) δ 10.40 (s, 1H), 8.39 (d, J=8.8 Hz, 1H), 7.58 (d, J=8.4 Hz, 2H), 7.32 (d, J=8.4 Hz, 2H), 6.27 (d, J=9.2 Hz, 1H), 5.17-5.00 (m, 1H), 4.71 (s, 2H), 1.25 (d, J=6.2 Hz, 6H), 0.89 (s, 9H), 0.07 (s, 6H).

### Step 2: (4-(2-(2-Aminopyridin-3-yl)-5-isopropoxy-3H-imidazo[4,5-b]pyridin-3-yl)phenyl)methanol

To a mixture of N-(4-(((tert-butyldimethylsilyl)oxy)methyl)phenyl)-6-isopropoxy-3-nitropyridin-2-amine (700 mg, 1.68 mmol) and 2-aminopyridine-3-carbaldehyde (225 mg, 1.84 mmol) in DMSO (10 mL) was added Na₂S₂O₄ (730 mg, 4.19 mmol). The mixture was stirred at 100° C. for 12 hr. H₂O (50 mL) was added. The mixture was filtered and concentrated to give (4-(2-(2-aminopyridin-3-yl)-5-isopropoxy-3H-imidazo[4,5-b]pyridin-3-yl)phenyl)methanol (500 mg, yield: 79%) as a brown solid. MS: m/z=376.0 [M+H]⁺.

### Step 3: 3-(3-(4-(Chloromethyl)phenyl)-5-isopropoxy-3H-imidazo[4,5-b]pyridin-2-yl)pyridin-2-amine

To a mixture of (4-(2-(2-aminopyridin-3-yl)-5-isopropoxy-3H-imidazo[4,5-b]pyridin-3-yl)phenyl)methanol (450 mg, 1.20 mmol) in CH₂Cl₂ (4 mL) was added SOCl₂ (174 μL, 2.40 mmol), the mixture was stirred at 40° C. for 1 hr. The mixture was concentrated to give 3-(3-(4-(chloromethyl)phenyl)-5-isopropoxy-3H-imidazo[4,5-b]pyridin-2-yl)pyridin-2-amine (Intermediate 149, 450 mg, yield: 95%) as a yellow solid. MS: m/z=394.0 [M+H]⁺.

### Intermediate 150: 3-(3-(4-(Chloromethyl)phenyl)-5-(methoxy-d₃)-3H-imidazo[4,5-b]pyridin-2-yl)pyridin-2-amine

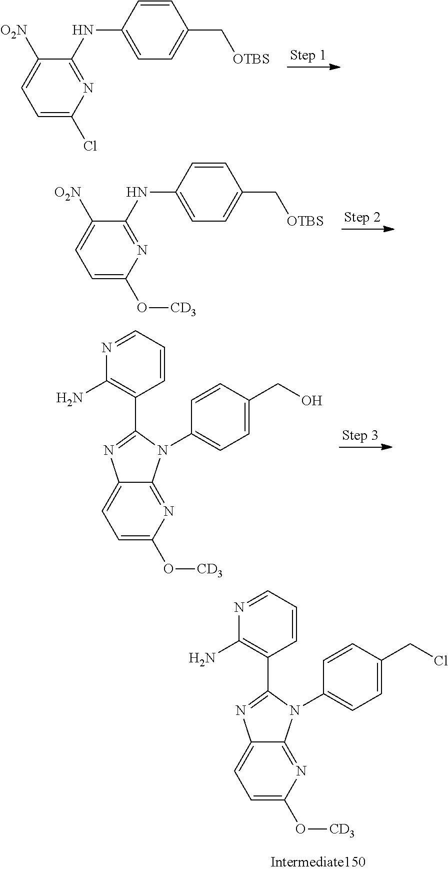

### Step 1: N-(4-(((tert-butyldimethylsilyl)oxy)methyl)phenyl)-6-(methoxy-d₃)-3-nitropyridin-2-amine

To a mixture of N-(4-(((tert-butyldimethylsilyl)oxy)methyl)phenyl)-6-chloro-3-nitropyridin-2-amine (refer to Intermediate 105 for detail procedures, 2 g, 5.08 mmol) in CD₃OD (30 mL) was added NaH (406 mg, 10.2 mmol, 60% purity) at 0° C. The mixture was stirred at 20° C. for 2 hr. H₂O (30 mL) was added slowly, and the mixture was extracted with EtOAc (30 mL×2). The combined organic layers were washed with brine (30 mL), dried over anhydrous Na₂SO₄, filtered and concentrated. N-(4-(((tert-butyldimethylsilyl)oxy)methyl)phenyl)-6-(methoxy-d₃)-3-nitropyridin-2-amine (1.77 g, yield: 89%) was obtained as a brown oil, which was used in the next step directly. MS: m/z=393.1 [M+H]⁺.

### Step 2: (4-(2-(2-Aminopyridin-3-yl)-5-(methoxy-d₃)-3H-imidazo[4,5-b]pyridin-3-yl)phenyl)methanol

To a solution of N-(4-(((tert-butyldimethylsilyl)oxy)methyl)phenyl)-6-(methoxy-d₃)-3-nitropyridin-2-amine (870 mg, 2.22 mmol) in DMSO (15 mL) were added Na₂S₂O₄ (1.16 g, 6.65 mmol) and 2-aminopyridine-3-carbaldehyde (298 mg, 2.44 mmol). The mixture was stirred at 100° C. for 12 hr. Sat. NaHCO₃ (50 mL) was added to the mixture and the mixture was extracted with CH₂Cl₂ (50 mL×2). The combined organic layers were washed with brine (50 mL), dried over anhydrous Na₂SO₄, filtered and concentrated. After purified by prep-HPLC (column: Xtimate C18 150×40 mm×10 μm; mobile phase: [water (NH₃H₂O+NH₄HCO₃)-ACN]; gradient: 26%-56% B over 8 min), (4-(2-(2-aminopyridin-3-yl)-5-(methoxy-d₃)-3H-imidazo[4,5-b]pyridin-3-yl)phenyl)methanol (200 mg, yield: 13%) was obtained as a yellow solid. MS: m/z=351.0 [M+H]+. ¹H NMR (400 MHz, Dimethylsulfoxide-d₆) δ 8.11 (d, J=8.8 Hz, 1H), 7.95 (d, J=2.8 Hz, 1H), 7.88 (d, J=4.0 Hz, 1H), 7.47-7.42 (m, 2H), 7.38-7.36 (m, 1H), 7.10 (dd, J=7.6, 2.0 Hz, 1H), 6.90-6.89 (m, 1H), 6.81 (d, J=8.8 Hz, 1H), 6.57 (dd, J=7.2, 4.8 Hz, 1H), 6.38 (dd, J=7.6, 4.8 Hz, 1H), 5.67-5.60 (m, 1H), 5.35 (t, J=5.6 Hz, 1H), 4.58 (d, J=6.0 Hz, 2H).

### Step 3: 3-(3-(4-(Chloromethyl)phenyl)-5-(methoxy-d₃)-3H-imidazo[4,5-b]pyridin-2-yl)pyridin-2-amine

To a solution of (4-(2-(2-aminopyridin-3-yl)-5-(methoxy-d₃)-3H-imidazo[4,5-b]pyridin-3-yl)phenyl)methanol (100 mg, 285 μmol) in CH₂Cl₂ (5 mL) was added SOCl₂ (102 mg, 856 μmol) at 20° C. The reaction mixture was stirred at 20° C. for 2 hr. The reaction mixture was concentrated to give 3-(3-(4-(Chloromethyl)phenyl)-5-(methoxy-d₃)-3H-imidazo[4,5-b]pyridin-2-yl)pyridin-2-amine (Intermediate 150, 105 mg, yield: 100¹%) as a yellow solid, which was used in the next step without further purification. MS: m/z=369.1 [M+H]⁺.

### Intermediate 151: 6-(2-(2-Aminopyridin-3-yl)-3-(4-(chloromethyl)phenyl)-3H-imidazo[4,5-b]pyridin-5-yl)picolinonitrile

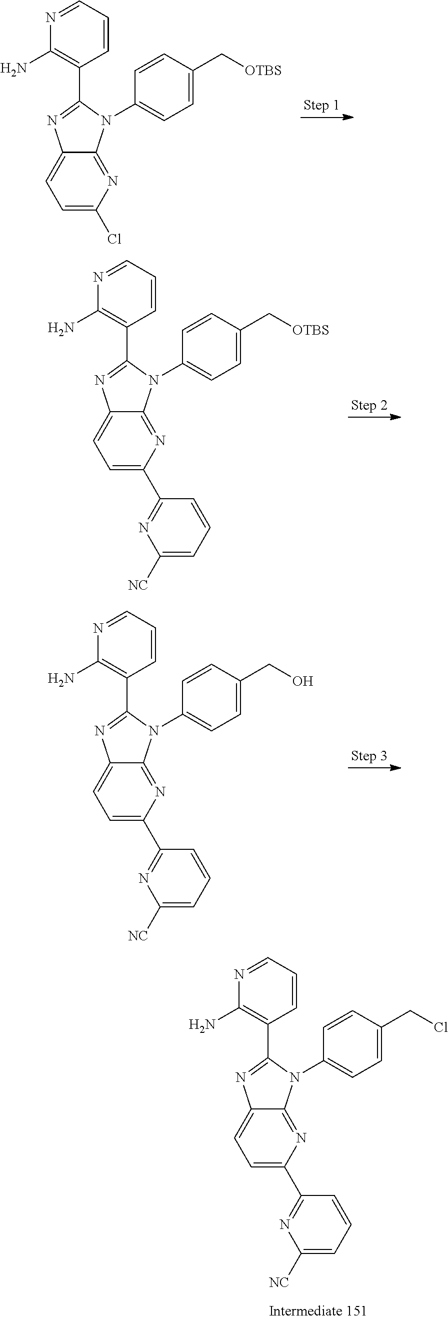

### Step 1: 6-(2-(2-Aminopyridin-3-yl)-3-(4-(((tert-butyldimethylsilyl)oxy)methyl)phenyl)-3H-imidazo[4,5-b]pyridin-5-yl)picolinonitrile

To a mixture of 3-(3-(4-(((tert-butyldimethylsilyl)oxy)methyl)phenyl)-5-chloro-3H-imidazo[4,5-b]pyridin-2-yl)pyridin-2-amine (refer to Intermediate 17 for detail procedures, 600 mg, 1.29 mmol) and 6-(4,4,5,5-Tetramethyl-1,3,2-dioxaborolan-2-yl)picolinonitrile (326 mg, 1.42 mmol) in 1,4-dioxane (10 mL) and H₂O (2 mL) were added CS₂CO₃ (1.26 g, 3.86 mmol) and Pd(dppf)Cl₂ (94.2 mg, 129 μmol). The mixture was degassed and purged with N₂ three times and stirred at 100° C. for 2 hr. H₂O (100 mL) was added, and the aqueous layer was extracted with EtOAc (200 mL×2). The combined organic layers were washed with brine (200 mL), dried over Na₂SO₄, filtered and concentrated. After purified by silica gel flash chromatography (Eluent of 0˜50%, EtOAc in petroleum ether), 6-(2-(2-aminopyridin-3-yl)-3-(4-(((tert-butyldimethylsilyl)oxy)methyl)phenyl)-3H-imidazo[4,5-b]pyridin-5-yl)picolinonitrile (500 mg, yield. 73%) was obtained as a yellow solid. MS: m/z=534.0 [M+H]⁺. ¹H NMR (400 MHz, Dimethylsulfoxide-d₆) δ 8.45-8.41 (m, 2H), 8.38-8.33 (m, 1H), 8.15 (t, J=7.6 Hz, 1H), 8.07-7.99 (m, 2H), 7.54-7.41 (m, 4H), 7.22 (dd, J=7.6, 1.6 Hz, 1H), 7.01 (br s, 2H), 6.40 (dd, J=7.6, 4.8 Hz, 1H), 4.83 (s, 2H), 0.93 (s, 9H), 0.11 (s, 6H).

### Step 2: (4-(2-(2-Aminopyridin-3-yl)-5-isopropoxy-3H-imidazo[4,5-b]pyridin-3-yl)phenyl)methanol

To a mixture of 6-(2-(2-aminopyridin-3-yl)-3-(4-(((tert-butyldimethylsilyl)oxy)methyl)phenyl)-3H-imidazo[4,5-b]pyridin-5-yl)picolinonitrile (450 mg, 843 μmol) in THF (5 mL) was added TBAF (1 M, 1.26 mL). The mixture was stirred at 20° C. for 1 hr. H₂O (50 mL) was added, and the aqueous layer was extracted with EtOAc (100 mL×2). The combined organic layers were washed with brine (100 mL), dried over Na₂SO₄, filtered and concentrated to give 6-(2-(2-aminopyridin-3-yl)-3-(4-(hydroxymethyl)phenyl)-3H-imidazo[4,5-b]pyridin-5-yl)picolinonitrile (350 mg) as a brown solid. MS: m/z=420.2 [M+H]⁺. ¹H NMR (400 MHz, Dimethylsulfoxide-d₆) δ 8.49-8.40 (m, 2H), 8.38-8.31 (m, 1H), 8.16 (t, J=8.0 Hz, 1H), 8.09-7.98 (m, 2H), 7.53-7.44 (m, 4H), 7.25 (dd, J=7.6, 1.6 Hz, 1H), 6.97 (br s, 2H), 6.43 (dd, J=7.6, 4.8 Hz, 1H), 5.38 (t, J=5.6 Hz, 1H), 4.61 (d, J=5.6 Hz, 2H).

### Step 3: 6-(2-(2-Aminopyridin-3-yl)-3-(4-(chloromethyl)phenyl)-3H-imidazo[4,5-b]pyridin-5-yl)picolinonitrile

To a mixture of 6-(2-(2-aminopyridin-3-yl)-3-(4-(hydroxymethyl)phenyl)-3H-imidazo[4,5-b]pyridin-5-yl)picolinonitrile (350 mg, 834 μmol) in CH₂Cl₂ (5 mL) was added SOCl₂ (121 μL, 1.67 mmol), the mixture was stirred at 40° C. for 1 hr. The mixture was concentrated to give 6-(2-(2-aminopyridin-3-yl)-3-(4-(chloromethyl)phenyl)-3H-imidazo[4,5-b]pyridin-5-yl)picolinonitrile (Intermediate 151, 320 mg) as a yellow solid. MS: m/z=438.0 [M+H]⁺. ¹H NMR (400 MHz, Dimethylsulfoxide-d₄) δ 8.78-8.25 (m, 5H), 8.25-8.13 (m, 2H), 8.12-8.06 (m, 1H), 7.95 (dd, J=7.6, 1.6 Hz, 1H), 7.68-7.60 (m, 4H), 6.93 (dd, J=7.6, 5.2 Hz, 1H), 4.88 (s, 2H).

### Intermediate 152: 4-(2-(2-Aminopyridin-3-yl)-3-(4-(chloromethyl)phenyl)-3H-imidazo[4,5-b]pyridin-5-yl)picolinonitrile

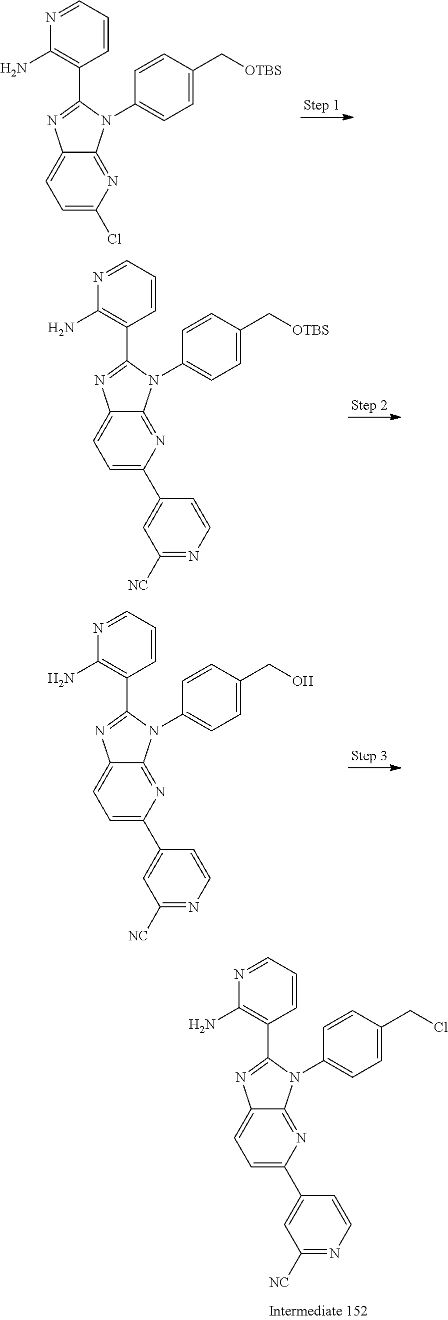

### Step 1: 4-(2-(2-Aminopyridin-3-yl)-3-(4-(((tert-butyldimethylsilyl)oxy)methyl)phenyl)-3H-imidazo[4,5-b]pyridin-5-yl)picolinonitrile

To a solution of 3-(3-(4-((tert-butyldimethylsilyl)oxy)methyl)phenyl)-5-chloro-3H-imidazo[4,5-b]pyridin-2-yl)pyridin-2-amine (refer to Intermediate 117 for detail procedures, 500 mg, 1.07 mmol) and 4-(4,4,5,5-tetramethyl-1,3,2-dioxaborolan-2-yl)pyridine-2-carbonitrile (272 mg, 1.18 mmol) in 1,4-dioxane (30 mL) and H₂O (6 mL) were added Pd(dppf)Cl₂ (78.5 mg, 107 μmol) and Cs₂CO₃ (1.05 g, 3.22 mmol) at 25° C. The mixture was degassed and purged with N₂ three times and stirred at 100° C. for 2 hr. The mixture was quenched with H₂O (50 mL) and the aqueous layer was extracted with EtOAc (50 mL×3). The combined organic layers were washed with brine (50 mL×3), dried over Na₂SO₄, filtered and concentrated. After purified by silica gel flash chromatography (Eluent of 0˜100% EtOAc in petroleum ether), 4-(2-(2-aminopyridin-3-yl)-3-(4-(((tert-butyldimethylsilyl)oxy)methyl)phenyl)-3H-imidazo[4,5-b]pyridin-5-yl)picolinonitrile (550 mg, yield: 96%) was obtained as a brown solid. MS: m/z=534.1 [M+1]⁺. ¹H NMR (400 MHz, Dimethylsulfoxide-d₆) δ 8.81 (d, J=5.2 Hz, 1H), 8.63-8.56 (m, 1H), 8.39 (d, J=8.4 Hz, 1H), 8.32 (dd, J=5.2, 1.6 Hz, 1H), 8.28 (d, J=8.4 Hz, 1H), 8.01 (dd, J=4.8, 2.0 Hz, 1H), 7.50 (br s, 4H), 7.22 (dd, J=7.6, 2.0 Hz, 1H), 7.12-6.97 (m, 2H), 6.40 (dd, J=7.6, 4.8 Hz, 1H), 4.83 (s, 2H), 0.92 (s, 9H), 0.10 (s, 6H).

### Step 2: 4-(2-(2-Aminopyridin-3-yl)-3-(4-(hydroxymethyl)phenyl)-3H-imidazo[4,5-b]pyridin-5-yl)picolinonitrile

To a solution of 4-(2-(2-aminopyridin-3-yl)-3-(4-(((tert-butyldimethylsilyl)oxy)methyl)phenyl)-3H-imidazo[4,5-b]pyridin-5-yl)picolinonitrile (500 mg, 937 μmol) in THF (15 mL) was added TBAF (1 M in THF, 1.41 mL) at 25° C. This mixture was stirred at 25° C. for 2 hr. The mixture was quenched with H₂O (30 ml) and the aqueous layer was extracted with EtOAc (30 mL×3). The combined organic layers were washed with brine (30 mL×2), dried over Na₂SO₄, filtered and concentrated to give 4-(2-(2-aminopyridin-3-yl)-3-(4-(hydroxymethyl)phenyl)-3H-imidazo[4,5-b]pyridin-5-yl)picolinonitrile (390 mg, crude) as a brown solid, which was used directly without purification. MS: m/z=420.2 [M+1]⁺.

### Step 3: 4-(2-(2-Aminopyridin-3-yl)-3-(4-(chloromethyl)phenyl)-3H-imidazo[4,5-b]pyridin-5-yl)picolinonitrile

To a solution of 4-(2-(2-aminopyridin-3-yl)-3-(4-(hydroxymethyl)phenyl)-3H-imidazo[4,5-b]pyridin-5-yl)picolinonitrile (390 mg, 930 μmol) in CH₂Cl₂ (5 mL) was added SOCl₂ (332 mg, 2.79 mmol) at 25° C. This mixture was stirred at 25° C. for 1 hr. The reaction mixture was concentrated to give 4-(2-(2-aminopyridin-3-yl)-3-(4-(chloromethyl)phenyl)-3H-imidazo[4,5-b]pyridin-5-yl)picolinonitrile (Intermediate 152, 400 mg, crude) as a brown solid, which was used in the next step without purification. MS: m/z=438.2 [M+1]˜.

### Intermediate 153: 5-(2-(2-Aminopyridin-3-yl)-3-(4-(chloromethyl)phenyl)-3H-imidazo[4,5-b]pyridin-5-yl)nicotinonitrile

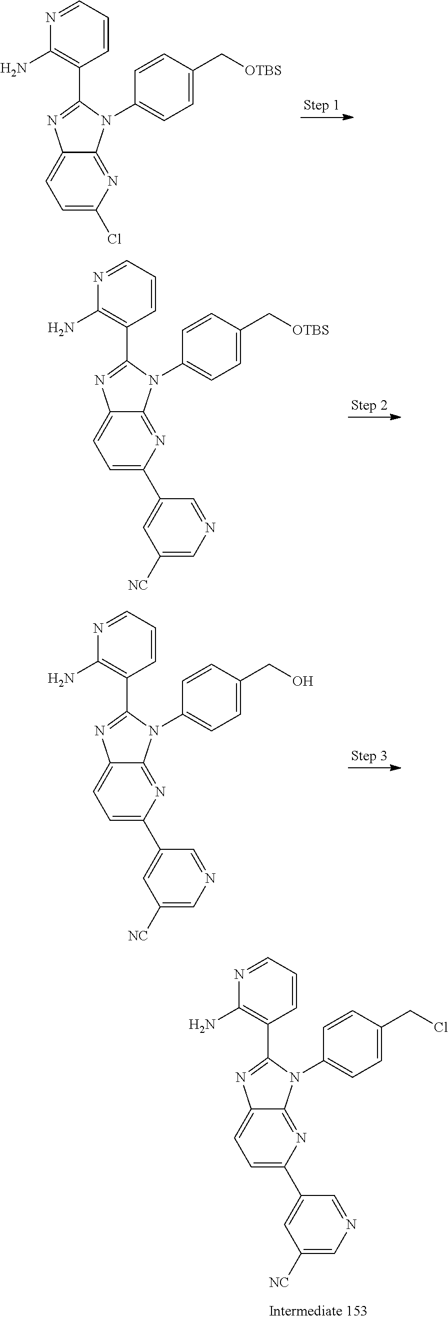

### Step 1: 5-(2-(2-Aminopyridin-3-yl)-3-(4-(((tert-butyldimethylsilyl)oxy)methyl)phenyl)-3H-imidazo[4,5-b]pyridin-5-yl)nicotinonitrile

To a solution of 3-(3-(4-(((tert-butyldimethylsilyl)oxy)methyl)phenyl)-5-chloro-3H-imidazo[4,5-b]pyridin-2-yl)pyridin-2-amine (refer to Intermediate 117 for detail procedures, 540 mg, 1.16 mmol) in 1,4-dioxane (30 mL) and H₂O (6 mL) were added (5-cyano-3-pyridyl)boronic acid (189 mg, 1.27 mmol), Cs₂CO₃ (1.13 g, 3.48 mmol) and Pd(dppf)Cl₂ (84.8 mg, 116 μmol) at 20° C. The reaction mixture was stirred at 100° C. for 2 hr. The reaction was quenched with H₂O (50 mL), and extracted with EtOAc (50 mL×2). The combined organic layers were washed with brine (50 mL), dried over anhydrous Na₂SO₄, filtered and concentrated. 5-(2-(2-aminopyridin-3-yl)-3-(4-(((tert-butyldimethylsilyl)oxy)methyl)phenyl)-3H-imidazo[4,5-b]pyridin-5-yl)nicotinonitrile (610 mg, crude) was obtained as a purple black oil, which was used in the next step without further purification. MS: m/z=534.1 [M+H]⁺.

### Step 2: 5-(2-(2-Aminopyridin-3-yl)-3-(4-(hydroxymethyl)phenyl)-3H-imidazo[4,5-b]pyridin-5-yl)nicotinonitrile

To a solution of 5-(2-(2-aminopyridin-3-yl)-3-(4-(((tert-butyldimethylsilyl)oxy)methyl)phenyl)-3H-imidazo[4,5-b]pyridin-5-yl)nicotinonitrile (310 mg, 581 μmol) in THF (12 mL) was added TBAF (1 M in THF) (1 M, 1.00 mL). The reaction mixture was stirred at 20° C. for 2 hr. H₂O (20 mL) was added and the mixture was extracted with EtOAc (25 mL×2). The combined organic layers were washed with brine (30 mL), dried over anhydrous Na₂SO₄, filtered and concentrated. 5-(2-(2-Aminopyridin-3-yl)-3-(4-(hydroxymethyl)phenyl)-3H-imidazo[4,5-b]pyridin-5-yl)nicotinonitrile (240 mg, crude) was obtained as a black solid, which was used in the next step without further purification. MS: m/z=420.2 [M+H]+.

### Step 3: 5-(2-(2-Aminopyridin-3-yl)-3-(4-(chloromethyl)phenyl)-3H-imidazo[4,5-b]pyridin-5-yl)nicotinonitrile

To a solution of 5-(2-(2-aminopyridin-3-yl)-3-(4-(hydroxymethyl)phenyl)-3H-imidazo[4,5-b]pyridin-5-yl)nicotinonitrile (240 mg, 572 μmol) in CH₂Cl₂ (8 mL) was added SOCl₂ (204 mg, 1.72 mmol) at 20° C. The reaction mixture was stirred at 40° C. for 1 hr. The reaction mixture was concentrated to give 5-(2-(2-aminopyridin-3-yl)-3-(4-(chloromethyl)phenyl)-3H-imidazo[4,5-b]pyridin-5-yl)nicotinonitrile (Intermediate 153, 250 mg, crude) as a brown solid, which was used in the next step directly. MS: m/z=438.1 [M+H]+.

### Intermediate 154: 3-(3-(4-(Chloromethyl)phenyl)-5-(pyridin-3-yl)-3H-imidazo[4,5-b]pyridin-2-yl)pyridin-2-amine

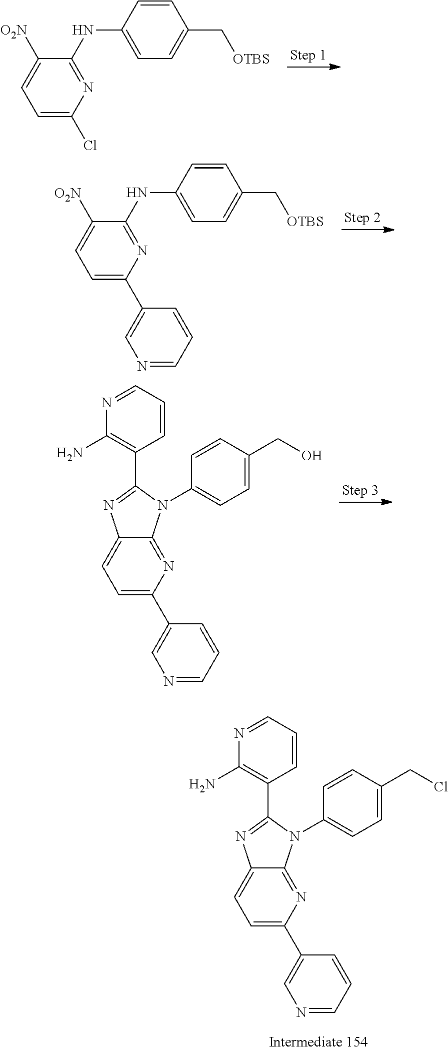

### Step 1: N-(4-(((Tert-butyldimethylsilyl)oxy)methyl)phenyl)-5-nitro-[2,3′-bipyridin]-6-amine

To a solution of N-(4-(((tert-butyldimethylsilyl)oxy)methyl)phenyl)-6-chloro-3-nitropyridin-2-amine (refer to Intermediate 105 for detail procedures, 2 g, 5.08 mmol) and 3-pyridylboronic acid (749 mg, 6.09 mmol) in 1,4-dioxane (20 mL) and H₂O (4 mL) were added Pd(dppf)Cl₂ (371 mg, 508 μmol) and Cs₂CO₃ (4.96 g, 15.2 mmol). The mixture was degassed and purged with N₂ three times, and then was stirred at 100° C. for 12 hr under N₂. H₂O (50 mL) was added, and the mixture was extracted with EtOAc (30 mL×2). The combined organic layers were dried over anhydrous Na₂SO₄, filtered and concentrated. N-(4-(((tert-butyldimethylsilyl)oxy)methyl)phenyl)-5-nitro-[2,3′-bipyridin]-6-amine (2.22 g) was obtained as a black brown oil, which was used in the next step directly. MS: m/z=437.2 [M+H]⁺.

### Step 2: (4-(2-(2-Aminopyridin-3-yl)-5-(pyridin-3-yl)-3H-imidazo[4,5-b]pyridin-3-yl)phenyl)methanol

To a solution of N-(4-(((tert-butyldimethylsilyl)oxy)methyl)phenyl)-5-nitro-[2,3′-bipyridin]-6-amine (2 g, 4.58 mmol) and 2-aminopyridine-3-carbaldehyde (615 mg, 5.04 mmol) in DMSO (20 mL) was added Na₂S₂O₄ (1.99 g, 11.5 mmol). The mixture was stirred at 100° C. for 12 hr under N₂. Sat. NaHCO₃ (50 mL) was added and the mixture was extracted with CH₂Cl₂/MeOH (10/1, 50 mL×3). The combined organic layers were washed with brine (50 mL×3), dried over Na₂SO₄, filtered and concentrated. After purified by silica gel flash chromatography (Eluent of 0˜100% EtOAc/EtOH (3/1) in petroleum ether), (4-(2-(2-aminopyridin-3-yl)-5-(pyridin-3-yl)-3H-imidazo[4,5-b]pyridin-3-yl)phenyl)methanol (500 mg, yield: 28% for 2 steps) was obtained as brown oil. MS: m/z=395.2 [M+H]⁺.

### Step 3: 3-(3-(4-(Chloromethyl)phenyl)-5-(pyridin-3-yl)-3H-imidazo[4,5-b]pyridin-2-yl)pyridin-2-amine

To a solution of (4-(2-(2-aminopyridin-3-yl)-5-(pyridin-3-yl)-3H-imidazo[4,5-b]pyridin-3-yl)phenyl)methanol (500 mg, 1.27 mmol) in CH₂Cl₂ (5 mL) was added SOCl₂ (452 mg, 3.80 mmol). The mixture was stirred at 40° C. for 1 hr under N₂. The solution was concentrated. 3-(3-(4-(Chloromethyl)phenyl)-5-(pyridin-3-yl)-3H-imidazo[4,5-b]pyridin-2-yl)pyridin-2-amine (Intermediate 154, 400 mg, yield: 76%) was obtained as yellow solid, which was used in the next step directly. MS: m/z=413.2 [M+H]⁺.

### Intermediate 155: 3-(3-(4-(Chloromethyl)phenyl)-5-(pyridin-4-yl)-3H-imidazo[4,5-b]pyridin-2-yl)pyridin-2-amine

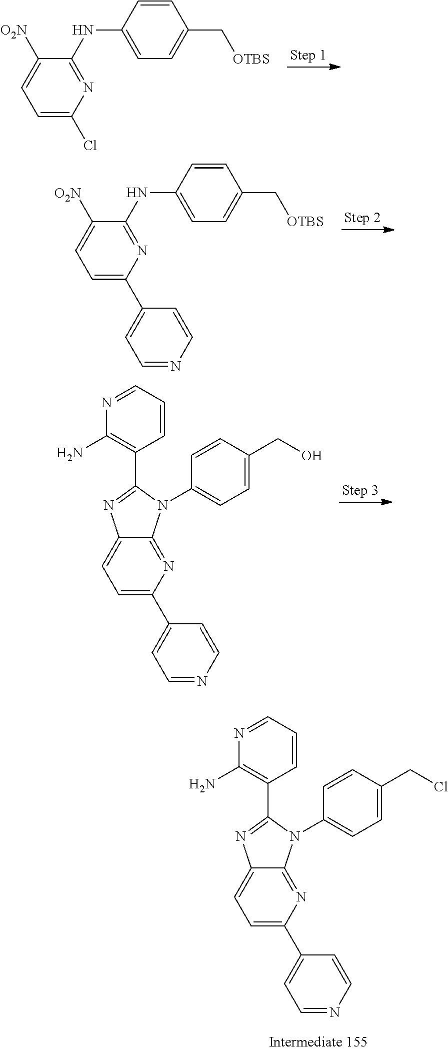

### Step 1: N-(4-(((tert-Butyldimethylsilyl)oxy)methyl)phenyl)-5-nitro-[2,4′-bipyridin]-6-amine

To a solution of N-(4-(((tert-butyldimethylsilyl)oxy)methyl)phenyl)-6-chloro-3-nitropyridin-2-amine (refer to Intermediate 105 for detail procedures, 2 g, 5.08 mmol) in 1,4-dioxane (20 mL) and H₂O (4 mL) were added 4-pyridylboronic acid (686 mg, 5.58 mmol), Pd(dppf)Cl₂ (371 mg, 507 μmol), and Cs₂CO₃ (4.96 g, 15.2 mmol). The mixture was degassed and purged with N₂ three times and was stirred at 100° C. for 2 hr under N₂ atmosphere. The crude was extracted with CH₂Cl₂ (30 mL×3). The combined organic layers were washed with brine (40 mL), dried over Na₂SO₄, filtered and concentrated. N-(4-(((tert-butyldimethylsilyl)oxy)methyl)phenyl)-5-nitro-[2,4′-bipyridin]-6-amine (2.22 g, crude) was obtained as a brown solid, which was used in the next step without further purification. MS: m/z=437.3 [M+H]⁺.

### Step 2: (4-(2-(2-Aminopyridin-3-yl)-5-(pyridin-4-yl)-3H-imidazo[4,5-b]pyridin-3-yl)phenyl)methanol

To a solution of N-(4-(((tert-butyldimethylsilyl)oxy)methyl)phenyl)-5-nitro-[2,4′-bipyridin]-6-amine (2.12 g, 4.86 mmol), 2-aminopyridine-3-carbaldehyde (711 mg, 5.83 mmol) in DMSO (20 mL) was added Na₂S₂O₄ (2.11 g, 12.1 mmol). The mixture was stirred at 100° C. for 12 hr. Sat. Na₂CO₃ (15 mL) was added to adjust pH about 8 and the mixture was extracted with CH₂Cl₂ (15 mL×3). The combined organic layers were washed with brine (30 mL), dried over Na₂SO₄, filtered and concentrated. After purified by silica gel flash chromatography (Eluent of 0˜78% EtOAc/EtOH (3:1) in petroleum ether), (4-(2-(2-aminopyridin-3-yl)-5-(pyridin-4-yl)-3H-imidazo[4,5-b]pyridin-3-yl)phenyl)methanol (100 mg, yield: 5% for 2 steps) was obtained as a yellow solid. MS: m4=395.3 [M+H]⁺.

### Step 3: 3-(3-(4-(Chloromethyl)phenyl)-5-(pyridin-4-yl)-3H-imidazo[4,5-b]pyridin-2-yl)pyridin-2-amine

To a solution of (4-(2-(2-aminopyridin-3-yl)-5-(pyridin-4-yl)-3H-imidazo[4,5-b]pyridin-3-yl)phenyl)methanol (100 mg, 253 μmol) in CH₂Cl₂ (2 mL) was added SOCl₂ (60.3 mg, 507 mol). The mixture was stirred at 25° C. for 2 hr. The residue was concentrated. 3-(3-(4-(Chloromethyl)phenyl)-5-(pyridin-4-yl)-3H-imidazo[4,5-b]pyridin-2-yl)pyridin-2-amine (Intermediate 155, 104 mg, crude) was obtained a brown solid, which was used in the next step without further purification. MS: m/z=413.2 [M+H]⁺.

### Intermediate 156: 7-(4-(2-(2-Aminopyridin-3-yl)-5-phenyl-3H-imidazo[4,5-b]pyridin-3-yl)benzyl)-7-azaspiro[3.5]nonan-2-amine

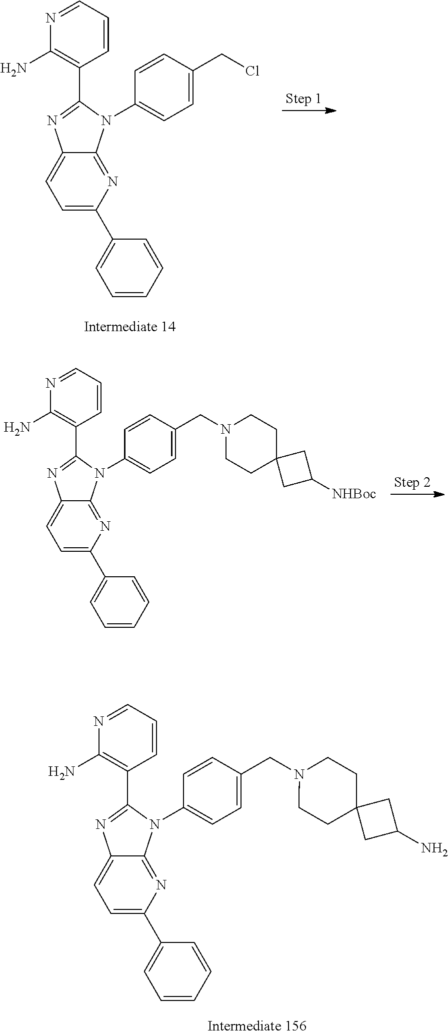 [SMILES: Nc1ncccc1-c1nc2ccc(-c3ccccc3)nc2n1-c1ccc(CCl)cc1 ; CC(C)(C)OC(=O)NC1CC2(CCN(Cc3ccc(-n4c(-c5cccnc5N)nc5ccc(-c6ccccc6)nc54)cc3)CC2)C1 ; Nc1ncccc1-c1nc2ccc(-c3ccccc3)nc2n1-c1ccc(CN2CCC3(CC2)CC(N)C3)cc1]

### Step 1 Tert-butyl (7-(4-(2-(2-aminopyridin-3-yl)-5-phenyl-3H-imidazo[4,5-b]pyridin-3-yl)benzyl)-7-azaspiro[3.5]nonan-2-yl)carbamate

To a solution of Intermediate 14 (1 g, 2.43 mmol) and tert-butyl 7-azaspiro[3.5]nonan-2-ylcarbamate (700 mg, 2.92 mmol) in ACN (20 mL) were added NaI (36.4 mg, 243 μmol) and K₂CO₃ (671 mg, 4.86 mmol). The mixture was stirred at 80° C. for 2 hr. The reaction mixture was concentrated. The residue was purified by silica gel flash chromatography (Eluent of 0˜10% MeOH in CH₂Cl₂) to give tert-butyl (7-(4-(2-(2-aminopyridin-3-yl)-5-phenyl-3H-imidazo[4,5-b]pyridin-3-yl)benzyl)-7-azaspiro[3.5]nonan-2-yl)carbamate (480 mg, yield: 32%,) as a light-yellow solid, which was used directly to the next step without further purification. MS: m/z=616.1 [M+H]⁺.

### Step 2: 7-(4-(2-(2-Aminopyridin-3-yl)-5-phenyl-3H-imidazo[4,5-b]pyridin-3-yl)benzyl)-7-azaspiro[3.5]nonan-2-amine

To a solution of tert-butyl N-[7-[[4-[2-(2-amino-3-pyridyl)-5-phenyl-imidazo[4,5-b]pyridin-3-yl]phenyl]methyl]-7-azaspiro[3.5]nonan-2-yl]carbamate (450 mg, 731 μmol) in HCl/1,4-dioxane (4 M, 6 mL). The mixture was stirred at 25° C. for 1 hr. The reaction mixture was concentrated. phase: [water (NH₄HCO₃)-ACN]; B %: 28%-58%, 8 min) to give 7-[[4-[2-(2-amino-3-pyridyl)-5-phenyl-imidazo[4,5-b]pyridin-3-yl]phenyl]methyl]-7-azaspiro[3.5]nonan-2-amine (Intermediate 156, 370 mg, yield: 91%) as a light-yellow oil. MS: m/z=516.3 [M+H]⁺. ¹H NMR (400 MHz, Dimethylsulfoxide-d₆) δ 8.27 (d, J=8.0 Hz, 1H), 8.06-7.95 (m, 4H), 7.55-7.35 (m, 7H), 7.18-7.12 (m, 1H), 7.01 (br s, 2H), 6.36 (dd, J=7.6, 4.8 Hz, 1H), 3.71-3.58 (m, 1H), 3.52 (s, 2H), 3.01-2.98 (m, 4H), 2.41-2.20 (m, 2H), 2.14-2.05 (m, 2H), 1.90-1.80 (m, 2H), 1.67-1.53 (m, 2H).

### Intermediate 157: 3-(3-(4-((2,7-Diazaspiro[4.4]nonan-2-yl)methyl)phenyl)-5-phenyl-3H-imidazo[4,5-b]pyridin-2-yl)pyridin-2-amine

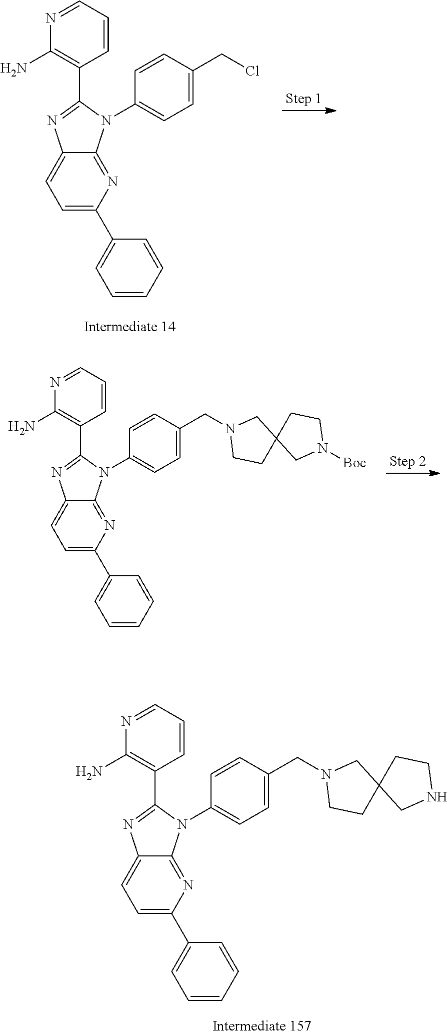 [SMILES: Nc1ncccc1-c1nc2ccc(-c3ccccc3)nc2n1-c1ccc(CCl)cc1 ; CC(C)(C)OC(=O)N1CCC2(CCN(Cc3ccc(-n4c(-c5cccnc5N)nc5ccc(-c6ccccc6)nc54)cc3)C2)C1 ; Nc1ncccc1-c1nc2ccc(-c3ccccc3)nc2n1-c1ccc(CN2CCC3(CCNC3)C2)cc1]

### Step 1. Tert-butyl 7-(4-(2-(2-aminopyridin-3-yl)-5-phenyl-3H-imidazo[4,5-b]pyridin-3-yl)benzyl)-2,7-diazaspiro[4.4]nonane-2-carboxylate

To a solution of Intermediate 14 (1.5 g, 3.6 mmol) and tert-butyl 2,7-diazaspiro[4.4]nonane-2-carboxylate (988 mg, 4.4 mmol) in DMF (10 mL) was added NaI (273 mg, 1.82 mmol) and K₂CO₃ (1.0 g, 7.3 mmol). The mixture was stirred at 80° C. for 1 hr. Then the reaction mixture was poured into H₂O (100 mL) and extracted with EtOAc (50 mL×2). The combined organic layers were washed with brine (50 mL×2), dried over anhydrous Na₂SO₄, filtered, and concentrated to dryness. The residue was purified by silica gel flash chromatography (Eluent of 0˜98% EtOAc in petroleum ether) to give tert-butyl 7-(4-(2-(2-aminopyridin-3-yl)-5-phenyl-3H-imidazo[4,5-b]pyridin-3-yl)benzyl)-2,7-diazaspiro[4.4]nonane-2-carboxylate (640 mg, yield: 30%) as a yellow solid. MS: m/z=602.4 [M+H]⁺

### Step 2: 3-(3-(4-((2,7-Diazaspiro[4.4]nonan-2-yl)methyl)phenyl)-5-phenyl-3H-imidazo[4,5-b]pyridin-2-yl)pyridin-2-amine

A solution of tert-butyl 7-(4-(2-(2-aminopyridin-3-yl)-5-phenyl-3H-imidazo[4,5-b]pyridin-3-yl)benzyl)-2,7-diazaspiro[4.4]nonane-2-carboxylate (590 mg, 980 μmol) in HCl in 1,4-dioxane (4 M, 5 mL) was stirred at 25° C. for 0.5 hr. The mixture was concentrated under reduced pressure to give 3-(3-(4-((2,7-diazaspiro[4.4]nonan-2-yl)methyl)phenyl)-5-phenyl-3H-imidazo[4,5-b]pyridin-2-yl)pyridin-2-amine (Intermediate 157, 452 mg, HCl salt, yield: 86%) as a yellow solid. MS: m/z=502.3 [M+H]⁺. ¹H NMR (400 MHz, Dimethylsulfoxide-d₆) δ 11.93 (br s, 1H), 9.88-9.48 (m, 2H), 8.60-8.35 (m, 2H), 8.38 (d, J=8.4 Hz, 1H), 8.16 (dd, J=6.4, 1.6 Hz, 1H), 8.08-8.05 (m, 3H), 7.98-7.83 (m, 3H), 7.66 (d, J=8.4 Hz, 2H), 7.52-7.40 (m, 3H), 7.02-6.92 (m, 1H), 4.54-4.43 (m, 2H), 3.64-3.47 (m, 2H), 3.43-3.27 (m, 3H), 3.25-3.18 (m, 3H), 2.31-2.14 (m, 2H), 2.11-1.96 (m, 2H).

### Intermediate 158: 3-(3-(4-((2,7-Diazaspiro[3.5]nonan-7-yl)methyl)phenyl)-5-phenyl-3H-imidazo[4,5-b]pyridin-2-yl)pyridin-2-amine

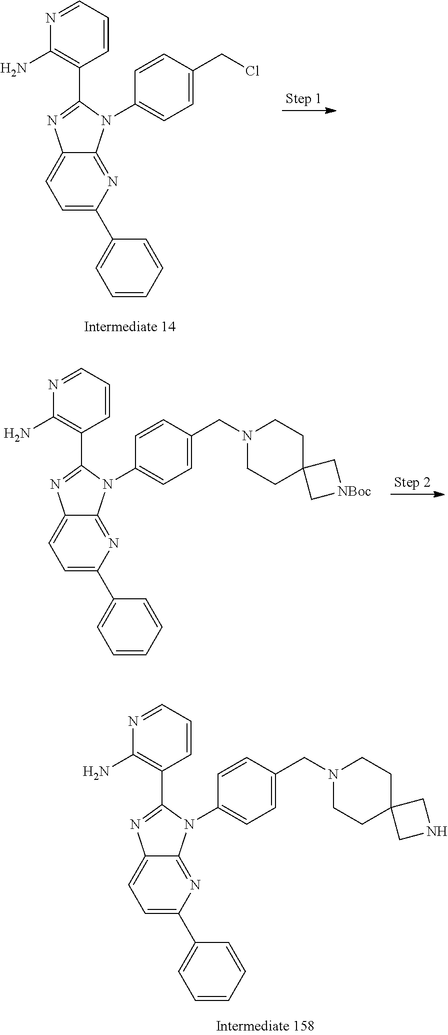 [SMILES: Nc1ncccc1-c1nc2ccc(-c3ccccc3)nc2n1-c1ccc(CCl)cc1 ; CC(C)(C)OC(=O)N1CC2(CCN(Cc3ccc(-n4c(-c5cccnc5N)nc5ccc(-c6ccccc6)nc54)cc3)CC2)C1 ; Nc1ncccc1-c1nc2ccc(-c3ccccc3)nc2n1-c1ccc(CN2CCC3(CC2)CNC3)cc1]

### Step 1: Tert-butyl 7-(4-(2-(2-aminopyridin-3-yl)-5-phenyl-3H-imidazo[4,5-b]pyridin-3-yl)benzyl)-2,7-diazaspiro[3.5]nonane-2-carboxylate

To a solution of Intermediate 14 (1.0 g, 2.43 mmol) and tert-butyl 2,7-diazaspiro[3.5]nonane-2-carboxylate (660 mg, 2.92 mmol) in ACN (20 mL) were added NaI (36.4 mg, 243 μmol) and K₂CO₃ (671 mg, 4.86 mmol). The mixture was stirred at 80° C. for 2 hr. The reaction mixture was concentrated. The residue was purified by silica gel flash chromatography (Eluent of 0˜10% MeOH in CH₂Cl₂) to give tert-butyl 7-(4-(2-(2-aminopyridin-3-yl)-5-phenyl-3H-imidazo[4,5-b]pyridin-3-yl)benzyl)-2,7-diazaspiro[3.5]nonane-2-carboxylate (570 mg, yield: 39%) as a light-yellow solid, which was used in the next step without further purification. MS: m/z=602.3 [M+H]⁺.

### Step 2: 3-(3-(4-((2,7-Diazaspiro[3.5]nonan-7-yl)methyl)phenyl)-5-phenyl-3H-imidazo[4,5-b]pyridin-2-yl)pyridin-2-amine

A solution of tert-butyl 7-(4-(2-(2-aminopyridin-3-yl)-5-phenyl-3H-imidazo[4,5-b]pyridin-3-yl)benzyl)-2,7-diazaspiro[3.5]nonane-2-carboxylate (490 mg, 814 μmol) in HCl in 1,4-dioxane (4 M, 9 mL) was stirred at 25° C. for 1 hr. The residue was concentrated and purified by prep-HPLC (column: Waters xbridge 150×25 mm 10 μm; mobile phase: [water (NH₄HCO₃)-ACN]; B %: 32%-62%, 8 min) to give 3-(3-(4-((2,7-diazaspiro[3.5]nonan-7-yl)methyl)phenyl)-5-phenyl-3H-imidazo[4,5-b]pyridin-2-yl)pyridin-2-amine (Intermediate 158, 300 mg, yield: 74%) as a light-yellow oil. MS: m/z=502.3 [M+H]+. ¹H NMR (400 MHz, Dimethylsulfoxide-d₆) δ 8.26 (d, J=8.4 Hz, 1H), 8.05-7.97 (m, 4H), 7.49-7.39 (m, 7H), 7.18-7.12 (dd, 1=7.6, 1.6 Hz, 1H), 7.02 (br s, 2H), 6.36 (dd, J=7.6, 5.2 Hz, 1H), 3.58-3.41 (m, 6H), 2.38-2.26 (m, 4H), 1.71-1.68 (m, 4H).

### Intermediate 159: 3-(3-(4-((2,7-Diazaspiro[4.5]decan-2-yl)methyl)phenyl)-5-phenyl-3H-imidazo[4,5-b]pyridin-2-yl)pyridin-2-amine

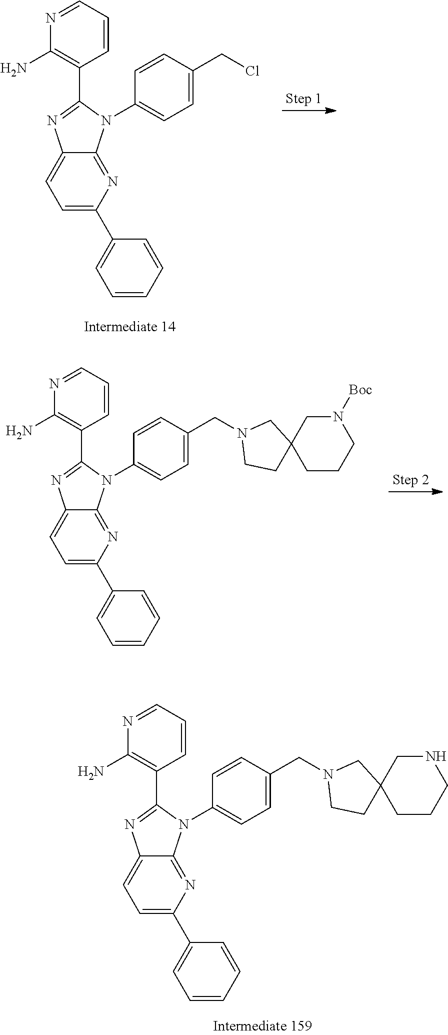 [SMILES: Nc1ncccc1-c1nc2ccc(-c3ccccc3)nc2n1-c1ccc(CCl)cc1 ; CC(C)(C)OC(=O)N1CCCC2(CCN(Cc3ccc(-n4c(-c5cccnc5N)nc5ccc(-c6ccccc6)nc54)cc3)C2)C1 ; Nc1ncccc1-c1nc2ccc(-c3ccccc3)nc2n1-c1ccc(CN2CCC3(CCCNC3)C2)cc1]

### Step 1: Tert-butyl 2-(4-(2-(2-aminopyridin-3-yl)-5-phenyl-3H-imidazo[4,5-b]pyridin-3-yl)benzyl)-2,7-diazaspiro[4.5]decane-7-carboxylate

To a solution of Intermediate 14 (1.5 g, 3.6 mmol) and tert-butyl 2,8-diazaspiro[4.5]decane-8-carboxylate (1.0 g, 4.4 mmol) in DMF (10 mL) were added NaI (273 mg, 1.8 mmol) and K₂CO₃ (1.5 g, 11 mmol). The mixture was stirred at 80° C. for 1 hr. After cooling to 20° C., the reaction mixture was poured into H₂O (100 mL) and extracted with EtOAc (50 mL×3). The combined organic layers were dried over anhydrous Na₂SO₄, filtered, and concentrated under reduced pressure. The residue was purified by silica gel flash chromatography (Eluent of 0˜85% EtOAc in petroleum ether) to give tert-butyl 2-(4-(2-(2-aminopyridin-3-yl)-5-phenyl-3H-imidazo[4,5-b]pyridin-3-yl)benzyl)-2,7-diazaspiro[4.5]decane-7-carboxylate (1.2 g, yield: 53%) as a yellow solid. MS: m/z=616.1 [M+H]⁺.

### Step 2: 3-(3-(4-((2,7-Diazaspiro[4.5]decan-2-yl)methyl)phenyl)-5-phenyl-3H-imidazo[4,5-b]pyridin-2-yl)pyridin-2-amine

A solution of tert-butyl 2-(4-(2-(2-aminopyridin-3-yl)-5-phenyl-3H-imidazo[4,5-b]pyridin-3-yl)benzyl)-2,7-diazaspiro[4.5]decane-7-carboxylate (240 mg, 390 μmol) in HCl/1,4-dioxane (4 M, 2 mL) was stirred at 25° C. for 0.5 hr. The mixture was concentrated and washed with CH₂Cl₂ (3 mL), then concentrated under reduced pressure. 3-(3-(4-((2,7-diazaspiro[4.5]decan-2-yl)methyl)phenyl)-5-phenyl-3H-imidazo[4,5-b]pyridin-2-yl)pyridin-2-amine (Intermediate 159, 188 mg, HCl salt, yield: 88%) was obtained as a yellow solid. MS: m/z=516.4 [M+H]⁺. ¹H NMR (400 MHz, Methanol-d₄) δ 8.32 (d, J=8.4 Hz, 1H), 8.09-8.00 (m, 4H), 7.93 (d, J=8.4 Hz, 2H), 7.89 (d, J=7.2 Hz, 1H), 7.72 (d, J=8.0 Hz, 2H), 7.49-7.37 (m, 3H), 6.99-6.85 (m, 1H), 4.62 (d, J=11.6 Hz, 2H), 3.78-3.72 (m, 1H), 3.65-3.47 (m, 2H), 3.44-3.38 (m, 1H), 3.35 (s, 2H), 3.18-3.13 (m, 2H), 2.26-2.13 (m, 1H), 2.11-2.01 (m, 1H), 1.95-1.86 (m, 4H).

### Intermediate 160: 3-(3-(4-((2,8-diazaspiro[4.5]decan-2-yl)methyl)phenyl)-5-phenyl-3H-imidazo[4,5-b]pyridin-2-yl)pyridin-2-amine

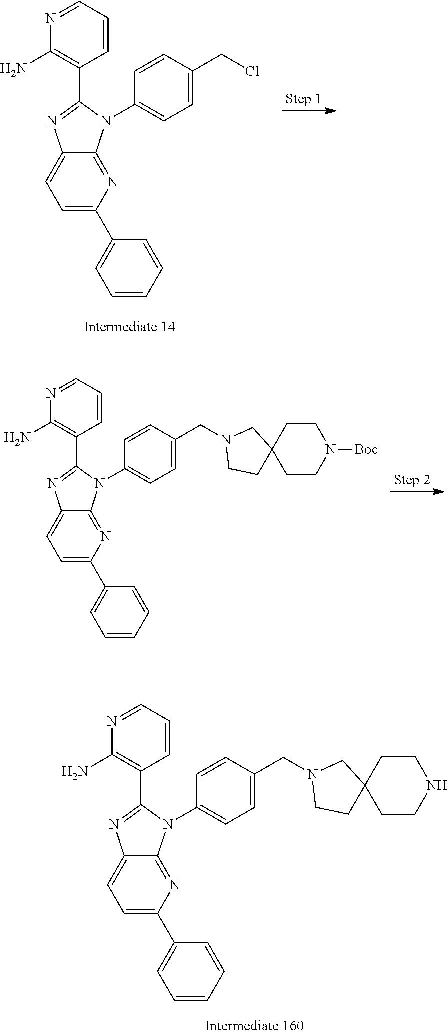 [SMILES: Nc1ncccc1-c1nc2ccc(-c3ccccc3)nc2n1-c1ccc(CCl)cc1 ; CC(C)(C)OC(=O)N1CCC2(CCN(Cc3ccc(-n4c(-c5cccnc5N)nc5ccc(-c6ccccc6)nc54)cc3)C2)CC1 ; Nc1ncccc1-c1nc2ccc(-c3ccccc3)nc2n1-c1ccc(CN2CCC3(CCNCC3)C2)cc1]

### Step 1: Tert-butyl 2-(4-(2-(2-aminopyridin-3-yl)-5-phenyl-3H-imidazo[4,5-b]pyridin-3-yl)benzyl)-2,8-diazaspiro[4.5]decane-8-carboxylate

To a solution of Intermediate 14 (1.5 g, 3.6 mmol) and tert-butyl 2,8-diazaspiro[4.5]decane-8-carboxylate (1.0 g, 4.4 mmol) in DMF (10 mL) were added NaI (273 mg, 1.8 mmol) and K₂CO₃ (1.0 g, 7.0 mmol). The mixture was stirred at 80° C. for 1 hr. After cooling to 20° C., the reaction mixture was poured into H₂O (100 mL), extracted with EtOAc (50 mL×3). The combined organic layers were dried over anhydrous Na₂SO₄, filtered, and concentrated under reduced pressure. The residue was purified by silica gel flash chromatography (Eluent of 0˜85% EtOAc in petroleum ether) to give tert-butyl 2-(4-(2-(2-aminopyridin-3-yl)-5-phenyl-3H-imidazo[4,5-b]pyridin-3-yl)benzyl)-2,8-diazaspiro[4.5]decane-8-carboxylate (720 mg, yield: 34%) as a yellow solid. MS: m/z=616.4 [M+H]⁺.

### Step 2: 3-(3-(4-(2,8-Diazaspiro[4.5]decan-2-ylmethyl)phenyl)-5-phenyl-3H-imidazo[4,5-b]pyridin-2-yl)pyridin-2-amine

A solution of tert-butyl 2-(4-(2-(2-aminopyridin-3-yl)-5-phenyl-3H-imidazo[4,5-b]pyridin-3-yl)benzyl)-2,8-diazaspiro[4.5]decane-8-carboxylate (720 mg, 1.2 mmol) in HCl in 1,4-dioxane (4 M, 5 mL) was stirred at 25° C. for 0.5 hr. The mixture was concentrated and washed with CH₂Cl₂ (3 mL), then concentrated under reduced pressure to give 3-(3-(4-((2,8-diazaspiro[4.5]decan-2-yl)methyl)phenyl)-5-phenyl-3H-imidazo[4,5-b]pyridin-2-yl)pyridin-2-amine (Intermediate 160, 600 mg, HCl salt, yield: 91%) as a yellow solid. MS: m/z=516.3 [M+H]⁺. ¹H NMR (400 MHz, Methanol-d₄) δ 8.33 (d, J=8.8 Hz, 1H), 8.10-8.01 (m, 4H), 7.96-7.87 (m, 3H), 7.72 (d, J=8.0 Hz, 2H), 7.49-7.40 (m, 3H), 6.96-6.87 (m, 1H), 4.67-4.54 (m, 2H), 3.75-3.65 (m, 2H), 3.51 (d, J=10.0 Hz, 1H), 3.35 (s, 2H), 3.32-3.20 (m, 3H), 2.32-2.23 (m, 1H), 2.15-2.05 (m, 2H), 2.04-1.96 (m, 3H).

### Intermediate 161: (R)-3-(3-(4-((2,7-Diazaspiro[4.4]nonan-2-yl)methyl)phenyl)-5-phenyl-3H-imidazo[4,5-b]pyridin-2-yl)pyridin-2-amine

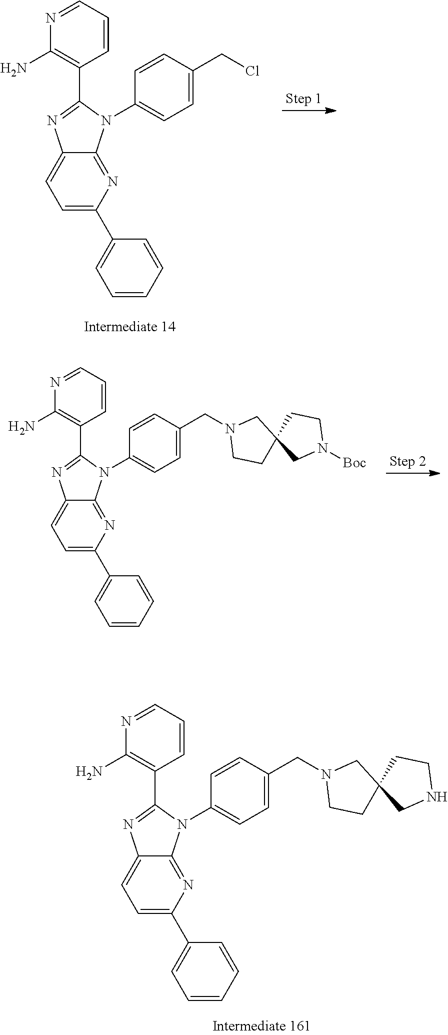 [SMILES: Nc1ncccc1-c1nc2ccc(-c3ccccc3)nc2n1-c1ccc(CCl)cc1 ; CC(C)(C)OC(=O)N1CC[C@@]2(CCN(Cc3ccc(-n4c(-c5cccnc5N)nc5ccc(-c6ccccc6)nc54)cc3)C2)C1 ; Nc1ncccc1-c1nc2ccc(-c3ccccc3)nc2n1-c1ccc(CN2CC[C@@]3(CCNC3)C2)cc1]

### Step 1; tert-Butyl (R)-7-(4-(2-(2-aminopyridin-3-yl)-5-phenyl-3H-imidazo[4,5-b]pyridin-3-yl)benzyl)-2,7-diazaspiro[4.4]nonane-2-carboxylate

To a solution of Intermediate 14 (1.0 g, 2.43 mmol) in DMF (10 mL) were added K₂CO₃ (671 mg, 4.86 mmol) and tert-butyl (R)-2,7-diazaspiro[4.4]nonane-2-carboxylate (604 mg, 2.67 mmol). The mixture was stirred at 25° C. for 16 hr. The reaction mixture was diluted with H₂O (10 mL) at 25° C. and extracted with CH₂Cl₂ (20 mL×3). The combined organic layers were washed with brine (30 ml), dried over Na₂SO₄, filtered and concentrated under reduced pressure. The crude product was purified by silica gel flash chromatography (Eluent of 1-5% MeOH in CH₂Cl₂) to give tert-butyl (R)-7-(4-(2-(2-aminopyridin-3-yl)-5-phenyl-3H-imidazo[4,5-b]pyridin-3-yl)benzyl)-2,7-diazaspiro[4.4]nonane-2-carboxylate (920 mg, yield: 63%) as a yellow solid. MS: m/z=602.6 [M+H]⁺.

### Step 2: (R)-3-(3-(4-((2,7-Diazaspiro[4.4]nonan-2-yl)methyl)phenyl)-5-phenyl-3H-imidazo[4,5-b]pyridin-2-yl)pyridin-2-amine

A solution of tert-butyl (R)-7-(4-(2-(2-aminopyridin-3-yl)-5-phenyl-3H-imidazo[4,5-b]pyridin-3-yl)benzyl)-2,7-diazaspiro[4.4]nonane-2-carboxylate (700 mg, 1.16 mmol) in 4 M HCl/1,4-dioxane (7 mL) was stirred at 25° C. for 2 hr. The reaction was concentrated under reduced pressure to give (R)-3-(3-(4-((2,7-diazaspiro[4.4]nonan-2-yl)methyl)phenyl)-5-phenyl-3H-imidazo[4,5-b]pyridin-2-yl)pyridin-2-amine (Intermediate 161, 600 mg, HCl salt, yield: 96%) as a yellow solid. MS: m/z=502.3 [M+H]⁺. ¹H NMR (400 MHz, Dimethylsulfoxide-d₆) δ 12.00-11.77 (m, 1H), 9.73-9.52 (m, 2H), 8.58-8.44 (m, 1H), 8.38 (d, J=8.4 Hz, 1H), 8.16 (dd, J=6.0, 1.6 Hz, 1H), 8.04-8.09 (m, 3H), 7.92-7.85 (m, 3H), 7.66 (d, 1=8.4 Hz, 2H), 7.52-7.46 (m, 2H), 7.45-7.40 (m, 1H), 7.00-6.92 (m, 1H), 4.57-4.41 (m, 2H), 3.50-3.58 (m, 4H), 3.41-3.33 (m, 2H), 3.25-3.22 (m, 2H), 2.28-2.13 (m, 2H), 2.10-1.97 (m, 2H).

### Intermediate 162: 3-(3-(4-(2,9-Diazaspiro[5.5]undecan-9-ylmethyl)phenyl)-5-phenyl-3H-imidazo[4,5-b]pyridin-2-yl)pyridin-2-amine

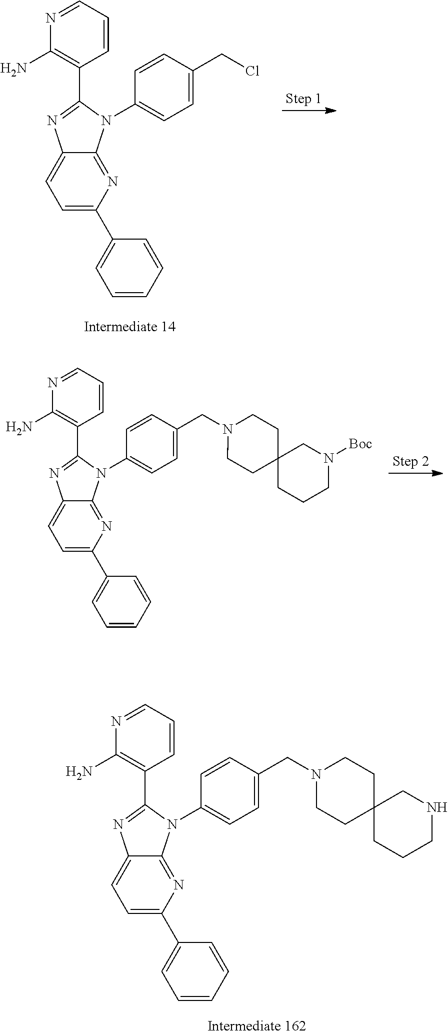 [SMILES: Nc1ncccc1-c1nc2ccc(-c3ccccc3)nc2n1-c1ccc(CCl)cc1 ; CC(C)(C)OC(=O)N1CCCC2(CCN(Cc3ccc(-n4c(-c5cccnc5N)nc5ccc(-c6ccccc6)nc54)cc3)CC2)C1 ; Nc1ncccc1-c1nc2ccc(-c3ccccc3)nc2n1-c1ccc(CN2CCC3(CCCNC3)CC2)cc1]

### Step 1: tert-Butyl 9-(4-(2-(2-aminopyridin-3-yl)-5-phenyl-3H-imidazo[4,5-b]pyridin-3-yl)benzyl)-2,9-diazaspiro[5.5]undecane-2-carboxylate

To a solution of Intermediate 14 in DMF (10 mL) were added K₂CO₃ (671 mg, 4.86 mmol) and tert-butyl 2,9-diazaspiro[5.5]undecane-2-carboxylate (679 mg, 2.67 mmol). The mixture was stirred at 25° C. for 48 hr. The reaction mixture was diluted with H₂O (10 ml) at 25° C. and extracted with CH₂Cl₂ (20 mL×3). The combined organic layers were washed with brine (15 mL), dried over Na₂SO₄, filtered and concentrated under reduced pressure. The crude was purified by silica gel flash chromatography (Eluent of 1˜4% MeOH in CH₂Cl₂) to give tert-butyl 9-(4-(2-(2-aminopyridin-3-yl)-5-phenyl-3H-imidazo[4,5-b]pyridin-3-yl)benzyl)-2,9-diazaspiro[5.5]undecane-2-carboxylat (612 mg, yield: 40%) as a yellow solid. MS: m/z=630.5 [M+H]⁺. ¹H NMR (400 MHz, Dimethylsulfoxide-d₆) 58.26 (d, J=8.4 Hz, 1H), 8.06-7.94 (m, 4H), 7.48-7.33 (m, 7H), 7.14 (dd, J=7.6, 1.6 Hz, 1H), 7.05 (br s, 2H), 6.35 (dd, J=7.2, 5.2 Hz, 1H), 3.62-3.55 (m, 2H), 3.27 (s, 2H), 3.22-3.15 (m, 2H), 2.49-2.44 (m, 2H), 2.35-2.22 (m, 2H), 1.38-1.34 (m, 8H), 1.38 (s, 1H).

### Step 2: 3-(3-(4-(2,9-Diazaspiro[5.5]undecan-9-ylmethyl)phenyl)-5-phenyl-3H-imidazo[4,5-b]pyridin-2-yl)pyridin-2-amine

A solution of tert-butyl 9-[[4-[2-(2-amino-3-pyridyl)-5-phenyl-imidazo[4,5-b]pyridin-3-yl]phenyl]methyl]-2,9-diazaspiro[5.5]undecane-2-carboxylate (630 mg, 1.0 mmol) in HCl/1,4-dioxane (4 M, 6 mL) was stirred at 25° C. for 1 hr. The reaction was concentrated under reduced pressure to give 3-(3-(4-(2,9-diazaspiro[5.5]undecan-9-ylmethyl)phenyl)-5-phenyl-3H-imidazo[4,5-b]pyridin-2-yl)pyridin-2-amine (Intermediate 162, 540 mg, HCl salt, 9.0 mg delivered) as a yellow solid. MS: m/z=530.4 [M+H]⁺. ¹H NMR (400 MHz, Dimethylsulfoxide-d₆) δ 11.24-10.95 (m, 1H), 9.45-8.99 (m, 2H), 8.52 (br s, 1H), 8.38 (d, J=8.4 Hz, 1H), 8.16 (d, 1=6.0 Hz, 1H), 8.12-8.02 (m, 3H), 7.91-7.81 (m, 3H), 7.73-7.62 (m, 2H), 7.52-7.40 (m, 3H), 6.93-6.84 (m, 1H), 4.41 (d, J=5.2 Hz, 2H), 3.21-3.09 (m, 5H), 2.99-2.90 (m, 2H), 2.87-2.75 (m, 1H), 2.13-2.09 (m, 1H), 2.00-1.88 (m, 1H), 1.86-1.73 (m, 2H), 1.72-1.56 (m, 3H), 1.45-1.40 (m, 1H).

### Intermediate 163: (S)-3-(3-(4-((2,7-Diazaspiro[4.4]nonan-2-yl)methyl)phenyl)-5-phenyl-3H-imidazo[4,5-b]pyridin-2-yl)pyridin-2-amine

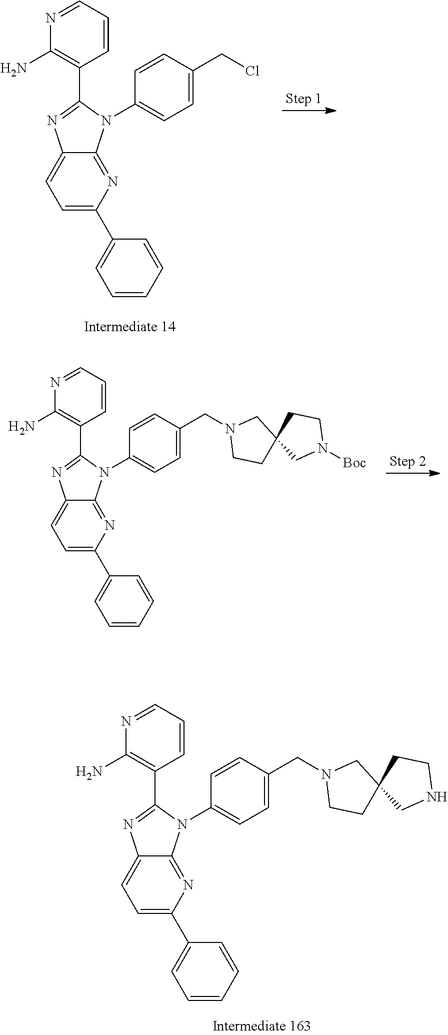 [SMILES: Nc1ncccc1-c1nc2ccc(-c3ccccc3)nc2n1-c1ccc(CCl)cc1 ; CC(C)(C)OC(=O)N1CC[C@]2(CCN(Cc3ccc(-n4c(-c5cccnc5N)nc5ccc(-c6ccccc6)nc54)cc3)C2)C1 ; Nc1ncccc1-c1nc2ccc(-c3ccccc3)nc2n1-c1ccc(CN2CC[C@]3(CCNC3)C2)cc1]

### Step 1. (S)-3-(3-(4-((2,7-Diazaspiro[4.4]nonan-2-yl)methyl)phenyl)-5-phenyl-3H-imidazo[4,5-b]pyridin-2-yl)pyridin-2-amine

To a solution of Intermediate 14 (1.0 g, 2.43 mmol) and tert-butyl (S)-2,7-diazaspiro[4.4]nonane-2-carboxylate (659 mg, 2.91 mmol) in DMF (15 mL) was added K₂CO₃ (671 mg, 4.86 mmol). The mixture was stirred at 25° C. for 16 hr. The reaction mixture was diluted with H₂O (50 mL) at 25° C. and extracted with EtOAc (100 mL×2). The combined organic layers were washed with brine (200 mL), dried over Na₂SO₄, filtered, and concentrated under reduced pressure. The residue was purified by silica gel flash chromatography (Eluent of 0˜5% MeOH in CH₂Cl₂) to give tert-butyl (S)-7-(4-(2-(2-aminopyridin-3-yl)-5-phenyl-3H-imidazo[4,5-b]pyridin-3-yl)benzyl)-2,7-diazaspiro[4.4]nonane-2-carboxylate (600 mg, yield: 41%) as a brown solid. MS: m/z=602.3 [M+H]⁺. ¹H NMR (400 MHz, Chloroform-d) δ 8.13 (d, J=8.4 Hz, 1H), 8.07 (dd, J=4.8, 1.6 Hz, 1H), 8.03-8.01 (m, 2H), 7.8 (d, J=8.4 Hz, 1H), 7.51-7.49 (m, 2H), 7.46-7.35 (m, 5H), 7.09 (dd, J=7.6, 1.6 Hz, 1H), 6.59 (br s, 2H), 6.36 (dd, J=7.6, 4.8 Hz, 1H), 3.80-3.70 (m, 2H), 3.50-3.20 (m, 4H), 2.80-2.50 (m, 4H), 1.95-1.80 (m, 4H), 1.46 (s, 9H).

### Step 2: (S)-3-(3-(4-((2,7-Diazaspiro[4.4]nonan-2-yl)methyl)phenyl)-5-phenyl-3H-imidazo[4,5-b]pyridin-2-yl)pyridin-2-amine

To a solution of tert-butyl (S)-7-(4-(2-(2-aminopyridin-3-yl)-5-phenyl-3H-imidazo[4,5-b]pyridin-3-yl)benzyl)-2,7-diazaspiro[4.4]nonane-2-carboxylate (580 mg, 964 μmol) in 1,4-dioxane (2 mL) was added HCl/1,4-dioxane (4 M, 8 mL). The mixture was stirred at 25° C. for 2 hr. The reaction was filtered and concentrated under reduced pressure. (S)-3-(3-(4-((2,7-Diazaspiro[4.4]nonan-2-yl)methyl)phenyl)-5-phenyl-3H-imidazo[4,5-b]pyridin-2-yl)pyridin-2-amine (Intermediate 163, 500 mg, HCl salt, yield: 96%) was obtained as a yellow solid. MS: m/z=502.3 [M+H]⁺. ¹H NMR (400 MHz, Dimethylsulfoxide-d₆) δ 11.9 (br s, 1H), 9.80-9.66 (m, 2H), 8.66-8.50 (m, 2H), 8.38 (d, J=8.4 Hz, 1H), 8.17 (dd, J=6.4, 1.6 Hz, 1H), 8.08-8.05 (m, 3H), 7.93-7.86 (M, 3H), 7.66 (d, J=8.4 Hz, 2H), 7.54-7.40 (m, 3H), 7.00-6.95 (m, 1H), 3.55-3.50 (m, 1H), 3.40-3.17 (m, 8H), 2.33-1.95 (m, 5H).

### Intermediate 164: (R)-3-(3-(4-((3-Methylpiperazin-1-yl)methyl)phenyl)-5-phenyl-3H-imidazo[4,5-b]pyridin-2-yl)pyridin-2-amine

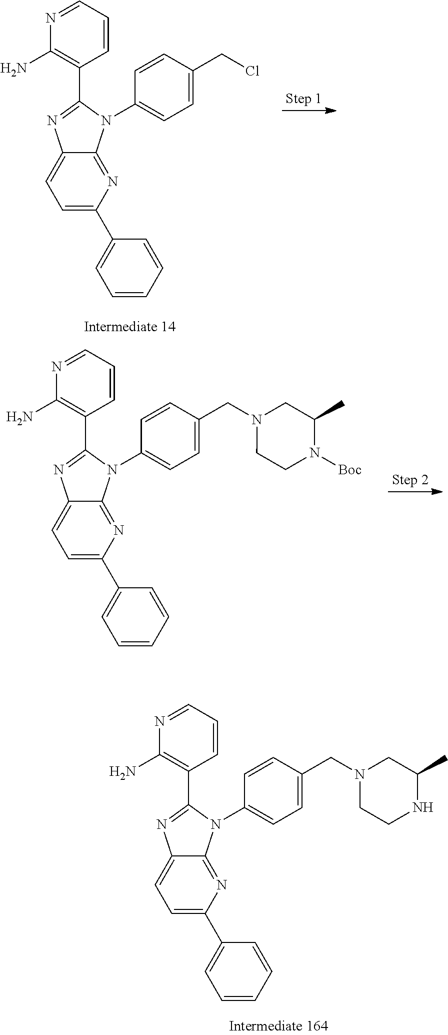 [SMILES: Nc1ncccc1-c1nc2ccc(-c3ccccc3)nc2n1-c1ccc(CCl)cc1 ; C[C@@H]1CN(Cc2ccc(-n3c(-c4cccnc4N)nc4ccc(-c5ccccc5)nc43)cc2)CCN1C(=O)OC(C)(C)C ; C[C@@H]1CN(Cc2ccc(-n3c(-c4cccnc4N)nc4ccc(-c5ccccc5)nc43)cc2)CCN1]

### Step 1: (R)-Tert-butyl 4-(4-(2-(2-aminopyridin-3-yl)-5-phenyl-3H-imidazo[4,5-b]pyridin-3-yl)benzyl)-2-methylpiperazine-1-carboxylate

To a solution of Intermediate 14 (1.0 g, 2.43 mmol) in DMF (5 mL) were added K₂CO₃ (671 mg, 4.86 mmol) and (R)-tert-butyl 2-methylpiperazine-1-carboxylate (632 mg, 3.16 mmol). The mixture was stirred at 25° C. for 16 hr. The reaction mixture was diluted with H₂O (10 mL) at 25° C. and extracted with CH₂Cl₂ (20 mL×3). The combined organic layers were washed with brine (30 mL), dried over Na₂SO₄, filtered, and concentrated under reduced pressure. The crude was purified by silica gel flash chromatography (Eluent of 1˜4% MeOH in CH₂Cl₂) to give (R)-tert-butyl 4-(4-(2-(2-aminopyridin-3-yl)-5-phenyl-3H-imidazo[4,5-b]pyridin-3-yl)benzyl)-2-methylpiperazine-1-carboxylate (671 mg, yield: 48%) as a yellow solid. MS: m/z=576.3 [M+H]+. ¹H NMR (400 MHz, Dimethylsulfoxide-d₆) 58.27 (d, J=8.4 Hz, 1H), 8.04-7.95 (m, 4H), 7.51-7.38 (m, 7H), 7.16 (dd, J=7.6, 1.6 Hz, 1H), 7.04 (br s, 2H), 6.38 (dd, J=8.0, 4.8 Hz, 1H), 4.14-4.07 (m, 1H), 3.72-3.70 (m, 1H), 3.64 (d, J=13.6 Hz, 1H), 3.49 (d, J=13.6 Hz, 1H), 3.09-2.98 (m, 1H), 2.84-2.78 (m, 1H), 2.63-2.57 (m, 1H), 2.08 (dd, J=11.2, 4.0 Hz, 1H), 2.02-1.92 (m, 1H), 1.40 (s, 9H), 1.17 (d, J=6.8 Hz, 3H).

### Step 2: (R)-3-(3-(4-((3-Methylpiperazin-1-yl)methyl)phenyl)-5-phenyl-3H-imidazo[4,5-b]pyridin-2-yl)pyridin-2-amine

To a solution of (R)-tert-butyl 4-(4-(2-(2-aminopyridin-3-yl)-5-phenyl-3H-imidazo[4,5-b]pyridin-3-yl)benzyl)-2-methylpiperazine-1-carboxylate (700 mg, 1.22 mmol) in 4 M HCl in 1,4-dioxane (5 mL) at 25° C. The mixture was stirred at 25° C. for 2 hr. The reaction was concentrated under reduced pressure to give product (500 mg, HCl salt) as a yellow solid. The product (50 mg, HCl salt) was diluted with aqueous NaHCO₃ (10 mL) and extracted with CH₂Cl₂ (10 mL×3). The combined organic layers were washed with brine (20 mL), dried over Na₂SO₄, filtered and concentrated under reduced pressure. (R)-3-(3-(4-((3-Methylpiperazin-1-yl)methyl)phenyl)-5-phenyl-3H-imidazo[4,5-b]pyridin-2-yl)pyridin-2-amine (Intermediate 164, 22.9 mg, yield: 86%) was obtained as a white solid. MS: m/z=476.2 [M+H]⁺. ¹H NMR (400 MHz, Dimethylsulfoxide-d₆) δ 8.27 (d, J=8.4 Hz, 1H), 8.08-7.95 (m, 4H), 7.52-7.35 (m, 7H), 7.15 (d, J=6.0 Hz, 1H), 7.04 (br s, 2H), 6.40-6.34 (m, 1H), 3.53 (s, 2H), 2.87-2.77 (m, 1H), 2.75-2.62 (m, 4H), 2.02-1.91 (m, 1H), 1.68-1.58 (m, 1H), 0.92 (d, J=6.0 Hz, 3H).

### Intermediate 165: 3-(3-(4-(((3S,5R)-3,5-Dimethylpiperazin-1-yl)methyl)phenyl)-5-phenyl-3H-imidazo[4,5-b]pyridin-2-yl)pyridin-2-amine

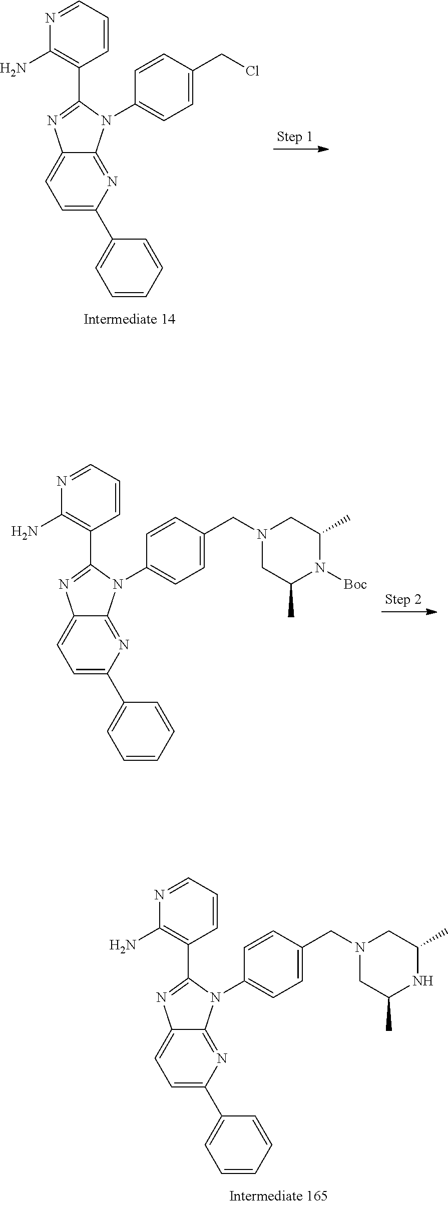 [SMILES: Nc1ncccc1-c1nc2ccc(-c3ccccc3)nc2n1-c1ccc(CCl)cc1 ; C[C@H]1CN(Cc2ccc(-n3c(-c4cccnc4N)nc4ccc(-c5ccccc5)nc43)cc2)C[C@H](C)N1C(=O)OC(C)(C)C ; C[C@H]1CN(Cc2ccc(-n3c(-c4cccnc4N)nc4ccc(-c5ccccc5)nc43)cc2)C[C@H](C)N1]

### Step 1: (2S,6R)-Tert-butyl 4-(4-(2-(2-aminopyridin-3-yl)-5-phenyl-3H-imidazo[4,5-b]pyridin-3-yl)benzyl)-2,6-dimethylpiperazine-1-carboxylate

To a solution of Intermediate 14 (1.0 g, 2.43 mmol) and tert-butyl (2S,6R)-2,6-dimethylpiperazine-1-carboxylate (520 mg, 2.43 mmol) in DMF (10 mL) was added K₂CO₃ (671 mg, 4.86 mmol). The mixture was stirred at 25° C. for 16 hr. The reaction mixture was diluted with H₂O (10 mL) at 25° C. and extracted with EtOAc (10 mL×3). The combined organic layers were washed with brine (10 mL×3), dried over Na₂SO₄, filtered, and concentrated under reduced pressure. The residue was purified by silica gel flash chromatography (Eluent of 1˜2% MeOH in CH₂Cl₂) to give (2S,6R)-tert-butyl 4-(4-(2-(2-aminopyridin-3-yl)-5-phenyl-3H-imidazo[4,5-b]pyridin-3-yl)benzyl)-2,6-dimethylpiperazine-1-carboxylate (770 mg, yield: 54%) as a yellow solid, which was used directly in the next step. MS: m/z=590.4 [M+H]⁺.

### Step 2: 3-(3-(4-(((3S,5R)-3,5-Dimethylpiperazin-1-yl)methyl)phenyl)-5-phenyl-3H-imidazo[4,5-b]pyridin-2-yl)pyridin-2-amine

To a solution of tert-butyl (2S,6R)-4-[[4-[2-(2-amino-3-pyridyl)-5-phenyl-imidazo[4,5-b]pyridin-3-yl]phenyl]methyl]-2,6-dimethyl-piperazine-1-carboxylate (50 mg, 84.9 μmol) in 1,4-dioxane (3 mL) was added HCl/1,4-dioxane (4 M, 5 mL). The mixture was stirred at 25° C. for 1 hr. The reaction mixture was concentrated under reduced pressure to remove 1,4-dioxane. The crude was dissolved in H₂O (5 mL) and extracted with CH₂Cl₂ (5 mL×3). The aqueous phase was added NaHCO₃ and extracted with CH₂Cl₂ (5 mL×3), filtered and concentrated to give 3-(3-(4-(((3S,5R)-3,5-dimethylpiperazin-1-yl)methyl)phenyl)-5-phenyl-3H-imidazo[4,5-b]pyridin-2-yl)pyridin-2-amine (Intermediate 165, 34 mg, yield: 82%) as a yellow solid. MS: m/z=490.1 [M+H]f ¹H NMR (400 MHz, Dimethylsulfoxide-d₆) δ 8.27 (d, J=8.4 Hz, 1H), 7.97-8.04 (m, 4H), 7.42-7.48 (m, 6H), 7.37-7.42 (m, 1H), 7.15 (dd, J=7.6, 2.0 Hz, 1H), 7.05 (br s, 2H), 6.36 (dd, J=7.6, 4.8 Hz, 1H), 3.52 (s, 2H), 2.73-2.79 (m, 2H), 2.64-2.67 (m, 2H), 1.56-1.50 (m, 2H), 0.91 (d, J=6.0 Hz, 6H).

### Intermediate 166: 4-(Azepan-4-ylamino)-1,3,5-triazine-2-carbonitrile

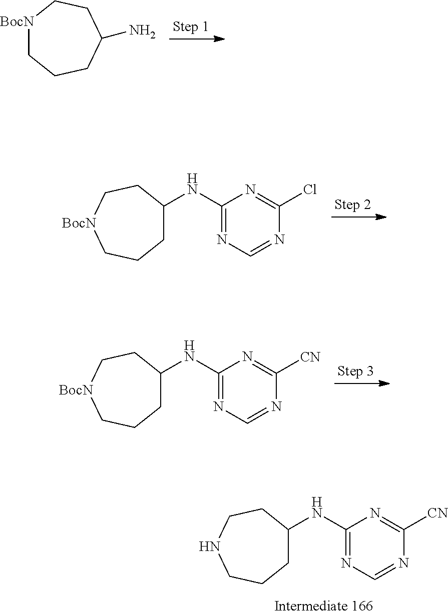 [SMILES: CC(C)(C)OC(=O)N1CCCC(N)CC1 ; CC(C)(C)OC(=O)N1CCCC(Nc2ncnc(Cl)n2)CC1 ; CC(C)(C)OC(=O)N1CCCC(Nc2ncnc(C#N)n2)CC1 ; N#Cc1ncnc(NC2CCCNCC2)n1]

### Step 1: Tert-butyl 4-((4-chloro-1,3,5-triazin-2-yl)amino)azepane-1-carboxylate

To a solution of tert-butyl 4-aminoazepane-1-carboxylate (500 mg, 2.33 mmol) and DIEA (905 mg, 7.00 mmol) in THF (20 mL) was added 2,4-dichloro-1,3,5-triazine (350 mg, 2.33 mmol). The mixture was stirred at 0° C. for 1 hr. The reaction mixture was diluted with H₂O (10 mL) and extracted with CH₂Cl₂ (10 mL×3). The combined organic layers were washed with brine (5 mL×2), dried over Na₂SO₄, filtered and concentrated under reduced pressure. The residue was purified by silica gel flash chromatography (Eluent of 10˜15% EtOAc in petroleum ether) to give tert-butyl 4-((4-chloro-1,3,5-triazin-2-yl)amino)azepane-1-carboxylate (300 mg, yield: 35%) as colorless oil. MS: m/z=328.1 [M+H]⁺.

### Step 2: tert-Butyl 4-((4-cyano-1,3,5-triazin-2-yl)amino)azepane-1-carboxylate

To a solution of tert-butyl 4-((4-chloro-1,3,5-triazin-2-yl)amino)azepane-1-carboxylate (300 mg, 915 μmol) in DMSO (10 mL) was added KCN (370 mg, 5.68 mmol). The mixture was stirred at 0° C. for 0.5 hr, then added DABCO (20.5 mg, 183 μmol). The mixture was stirred at 25° C. for 16 hr. The mixture was quenched with water (10 mL) and extracted with EtOAc (10 mL×3). The combined organic layers were dried over anhydrous Na₂SO₄, filtered and concentrated. The residue was purified by silica gel flash chromatography (Eluent of 5˜8% EtOAc in petroleum ether) to give tert-butyl 4-((4-cyano-1,3,5-triazin-2-yl)amino)azepane-1-carboxylate (240 mg, yield: 81%) as an off-white oil. MS: m/z=319.0 [M+H]⁺.

### Step 3: 4-(Azepan-4-ylamino)-1,3,5-triazine-2-carbonitrile

To a solution of tert-butyl 4-((4-cyano-1,3,5-triazin-2-yl)amino)azepane-1-carboxylate (240 mg, 754 μmol) in CH₂Cl₂ (5 mL) was added TFA (172 mg, 1.51 mmol). The mixture was stirred at 25° C. for 1 hr. The reaction mixture was filtered and concentrated under reduced pressure to give 4-(azepan-4-ylamino)-1,3,5-triazine-2-carbonitrile (Intermediate 166, 250 mg, yield: 100%) as a pink oil. The crude was directly used to the next step.

### Intermediate 167: 4-((1,4-Oxazepan-6-yl)amino)-1,3,5-triazine-2-carbonitrile

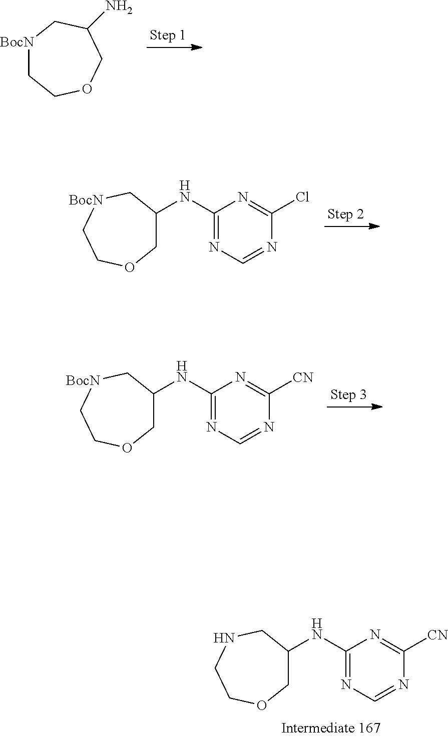 [SMILES: CC(C)(C)OC(=O)N1CCOCC(N)C1 ; CC(C)(C)OC(=O)N1CCOCC(Nc2ncnc(Cl)n2)C1 ; CC(C)(C)OC(=O)N1CCOCC(Nc2ncnc(C#N)n2)C1 ; N#Cc1ncnc(NC2CNCCOC2)n1]

### Step 1: Tert-butyl 6-((4-chloro-1,3,5-triazin-2-yl)amino)-1,4-oxazepane-4-carboxylate

To a solution of tert-butyl 6-amino-1,4-oxazepane-4-carboxylate (600 mg, 2.77 mmol) and 2,4-dichloro-1,3,5-triazine (416 mg, 2.77 mmol) in THF (10 mL) was added DIEA (685 mg, 5.3 mmol). The resulting mixture was stirred at 0° C. for 12 hr. Then the reaction mixture was diluted with H₂O (50 mL) and extracted with CH₂Cl₂ (50 mL×2). The combined organic layers were dried over Na₂SO₄, filtered and concentrated under reduced pressure. tert-Butyl 6-((4-chloro-1,3,5-triazin-2-yl)amino)-1,4-oxazepane-4-carboxylate (400 mg, yield: 39%) was obtained as a light yellow solid. MS: m/z=330.1 [M+H]⁺.

### Step 2: tert-Butyl 6-((4-cyano-1,3,5-triazin-2-yl)amino)-1,4-oxazepane-4-carboxylate

To a solution of tert-butyl 6-((4-chloro-1,3,5-triazin-2-yl)amino)-1,4-oxazepane-4-carboxylate (400 mg, 1.21 mmol) in DMSO (9 mL) were added KCN (260 mg, 3.99 mmol) and then DABCO (27.2 mg, 243 μmol). The mixture was stirred at 25° C. for 1 hr. The reaction mixture was poured into H₂O (50 mL). The resulting mixture was extracted with EtOAc (50 mL×2). The combined organic layers were washed with brine (50 mL), dried over Na₂SO₄, filtered and concentrated under reduced pressure. After purified by flash chromatography on silica gel (Eluent of 20%˜30% EtOAc in petroleum ether), tert-butyl 6-((4-cyano-1,3,5-triazin-2-yl)amino)-1,4-oxazepane-4-carboxylate (380 mg, yield: 63%) was obtained as a light yellow solid. MS: m/z=321.0 [M+H]⁺.

### Step 3: 4-((1,4-Oxazepan-6-yl)amino)-1,3,5-triazine-2-carbonitrile

To a solution of tert-butyl 6-((4-cyano-1,3,5-triazin-2-yl)amino)-1,4-oxazepane-4-carboxylate (250 mg, 780 μmol) in CH₂Cl₂ (3 mL) was added TFA (1.54 g, 13.5 mmol). The mixture was stirred at 25° C. for 0.5 hr. The reaction mixture was filtered and concentrated under reduced pressure to give 4-((1,4-oxazepan-6-yl)amino)-1,3,5-triazine-2-carbonitrile (Intermediate 167, 330 mg, crude) was obtained as a yellow oil, which was used directly in the next step.

### Intermediate 168: 3-(3-(4-((2,8-Diazaspiro[4.5]decan-8-yl)methyl)phenyl)-5-phenyl-3H-imidazo[4,5-b]pyridin-2-yl)pyridin-2-amine

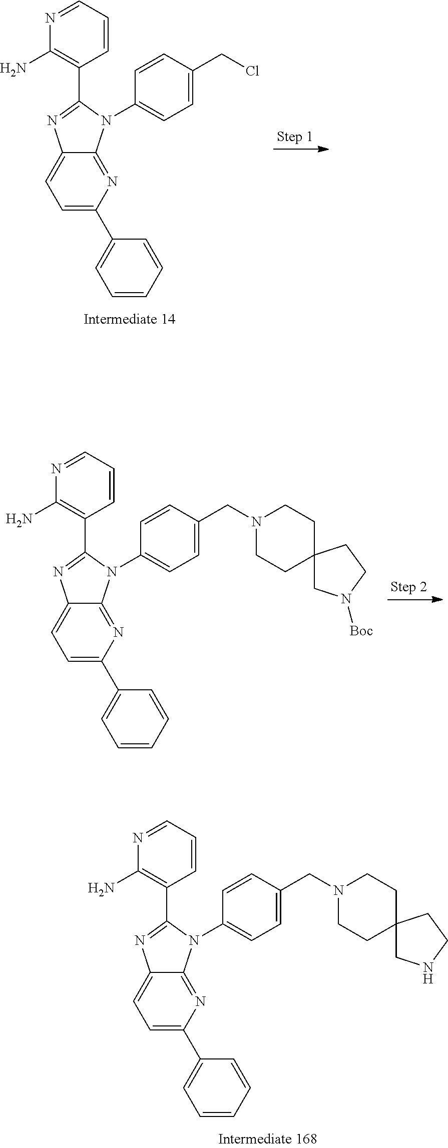 [SMILES: Nc1ncccc1-c1nc2ccc(-c3ccccc3)nc2n1-c1ccc(CCl)cc1 ; CC(C)(C)OC(=O)N1CCC2(CCN(Cc3ccc(-n4c(-c5cccnc5N)nc5ccc(-c6ccccc6)nc54)cc3)CC2)C1 ; Nc1ncccc1-c1nc2ccc(-c3ccccc3)nc2n1-c1ccc(CN2CCC3(CCNC3)CC2)cc1]

### Step 1 Tert-butyl 8-(4-(2-(2-aminopyridin-3-yl)-5-phenyl-3H-imidazo[4,5-b]pyridin-3-yl)benzyl)-2,8-diazaspiro[4.5]decane-2-carboxylate

To a solution of Intermediate 14 (250 mg, 607 μmol) in DMF (2 mL) were added tert-butyl 2,8-diazaspiro[4.5]decane-2-carboxylate (160 mg, 668 μmol), NaI (9.10 mg, 60.7 μmol) and K₂CO₃ (252 mg, 1.82 mmol). The resulting mixture was stirred at 80° C. for 16 hr. The reaction mixture was poured into H₂O (5 mL) and extracted with EtOAc (10 mL×3). The combined organic layers were dried over anhydrous Na₂SO₄, filtered, and concentrated under reduced pressure. The residue was purified by silica gel flash chromatography (Eluent of 0˜65% EtOAc in petroleum ether) to give tert-butyl 8-(4-(2-(2-aminopyridin-3-yl)-5-phenyl-3H-imidazo[4,5-b]pyridin-3-yl)benzyl)-2,8-diazaspiro[4.5]decane-2-carboxylate (200 mg, yield: 53%) as a yellow solid. MS: m/z=616.2 [M+H]⁺.

### Step 2: 3-(3-(4-((2,8-Diazaspiro[4.5]decan-8-yl)methyl)phenyl)-5-phenyl-3H-imidazo[4,5-b]pyridin-2-yl)pyridin-2-amine

To a solution of tert-butyl 8-(4-(2-(2-aminopyridin-3-yl)-5-phenyl-3H-imidazo[4,5-b]pyridin-3-yl)benzyl)-2,8-diazaspiro[4.5]decane-2-carboxylate (280 mg, 455 μmol) in dioxane (1 mL) was added 4 M HCl in 1,4-dioxane (2 mL). The mixture was stirred at 25° C. for 1 hr. The reaction mixture was concentrated under reduced pressure. Saturated NaHCO₃ (5 mL) aqueous solution was added and extracted with CH₂Cl₂ (15 mL). The organic layer was dried over Na₂SO₄, filtered, and concentrated under reduced pressure. (Intermediate 168, 200 mg, yield: 85%). MS: m/z=516.3 [M+H]⁺. ¹H NMR (400 MHz, Methanol-d₄) δ 8.18 (d, J=8.4 Hz, 1H), 8.02 (d, J=7.2 Hz, 2H), 7.97 (dd, J=4.8, 1.6 Hz, 1H), 7.93 (d, J=8.4 Hz, 1H), 7.52 (d, J=8.4 Hz, 2H), 7.45-7.34 (m, 5H), 7.31 (dd, J=6.8, 1.6 Hz, 1H), 6.45 (dd, J=7.6, 4.8 Hz, 1H), 3.62 (s, 2H), 3.10 (t, J=7.2 Hz, 2H), 2.85 (s, 2H), 2.50 (m, 4H), 1.75 (t, J=7.2 Hz, 2H), 1.65 (t, J=5.6 Hz, 4H).

### Intermediate 169: 3-(3-(4-((3,9-diazaspiro[5.5]undecan-3-yl)methyl)phenyl)-5-phenyl-3H-imidazo[4,5-b]pyridin-2-yl)pyridin-2-amine

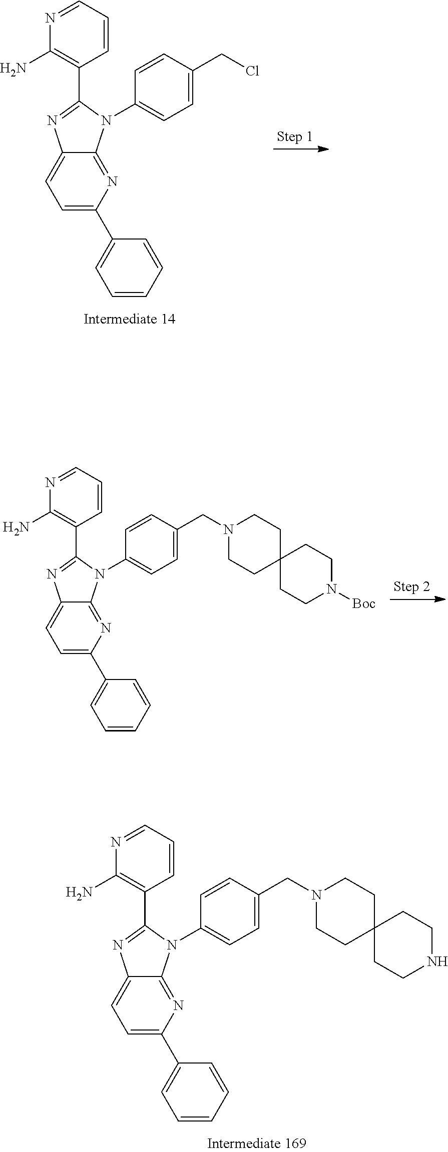 [SMILES: Nc1ncccc1-c1nc2ccc(-c3ccccc3)nc2n1-c1ccc(CCl)cc1 ; CC(C)(C)OC(=O)N1CCC2(CCN(Cc3ccc(-n4c(-c5cccnc5N)nc5ccc(-c6ccccc6)nc54)cc3)CC2)CC1 ; Nc1ncccc1-c1nc2ccc(-c3ccccc3)nc2n1-c1ccc(CN2CCC3(CCNCC3)CC2)cc1]

### Step 1 Tert-butyl 9-(4-(2-(2-aminopyridin-3-yl)-5-phenyl-3H-imidazo[4,5-b]pyridin-3-yl)benzyl)-3,9-diazaspiro[5.5]undecane-3-carboxylate

To a solution of Intermediate 14 (600 mg, 1.46 mmol) and tert-butyl 3,9-diazaspiro[5.5]undecane-3-carboxylate (408 mg, 1.60 mmol) in DMF (5 mL) were added NaI (21.9 mg, 146 μmol) and K₂CO₃ (403 mg, 2.91 mmol). The mixture was stirred at 80° C. for 16 hr. The reaction mixture was poured into H₂O (15 mL), extracted with EtOA (20 mL×3). The combined organic layers were dried over anhydrous Na₂SO₄, filtered and concentrated under reduced pressure. The residue was purified by silica gel flash chromatography (Eluent of 0˜65% EtOAc in petroleum ether), tert-butyl 9-(4-(2-(2-aminopyridin-3-yl)-5-phenyl-3H-imidazo[4,5-b]pyridin-3-yl)benzyl)-3,9-diazaspiro[5.5]undecane-3-carboxylate (450 mg, yield: 49%) was obtained as a yellow solid. MS: m/z=630.5 [M+H]⁺.

### Step 2: 3-(3-(4-((3,9-Diazaspiro[5.5]undecan-3-yl)methyl)phenyl)-5-phenyl-3H-imidazo[4,5-b]pyridin-2-yl)pyridin-2-amine

To a solution of 9-(4-(2-(2-aminopyridin-3-yl)-5-phenyl-3H-imidazo[4,5-b]pyridin-3-yl)benzyl)-3,9-diazaspiro[5.5]undecane-3-carboxylate (200 mg, 318 μmol) in 1,4-dioxane (2 mL) was added 4 M HCl in 1,4-dioxane (2 mL). The mixture was stirred at 25° C. for 1 hr. The reaction mixture was filtered, and the filter cake was dried. 3-(3-(4-((3,9-Diazaspiro[5.5]undecan-3-yl)methyl)phenyl)-5-phenyl-3H-imidazo[4,5-b]pyridin-2-yl)pyridin-2-amine (Intermediate 169, 121 mg, HCl salt, yield: 67%) was obtained as a yellow solid. MS: m/z=530.3 [M+H]⁺. ¹H NMR (400 MHz, Dimethylsulfoxide-d₆) δ 10.81-10.50 (m, 1H), 8.79-8.66 (m, 2H), 8.38 (dd, J=8.4 Hz, 1H), 8.13-8.11 (m, 1H), 8.08-8.05 (m, 4H), 7.86-7.77 (m, 3H), 7.67 (d, J=8.0 Hz, 2H), 7.51-7.46 (m, 2H), 7.45-7.41 (m, 1H), 6.86-6.78 (m, 1H), 4.41 (d, J=4.4 Hz, 2H), 3.21-3.15 (m, 2H), 3.09-3.01 (m, 6H), 1.91-1.85 (m, 2H), 1.82-1.71 (m, 4H), 1.54-1.53 (m, 2H).

### Intermediate 170: 3-(3-(4-((2,6-Diazaspiro[3.3]heptan-2-yl)methyl)phenyl)-5-phenyl-3H-imidazo[4,5-b]pyridin-2-yl)pyridin-2-amine

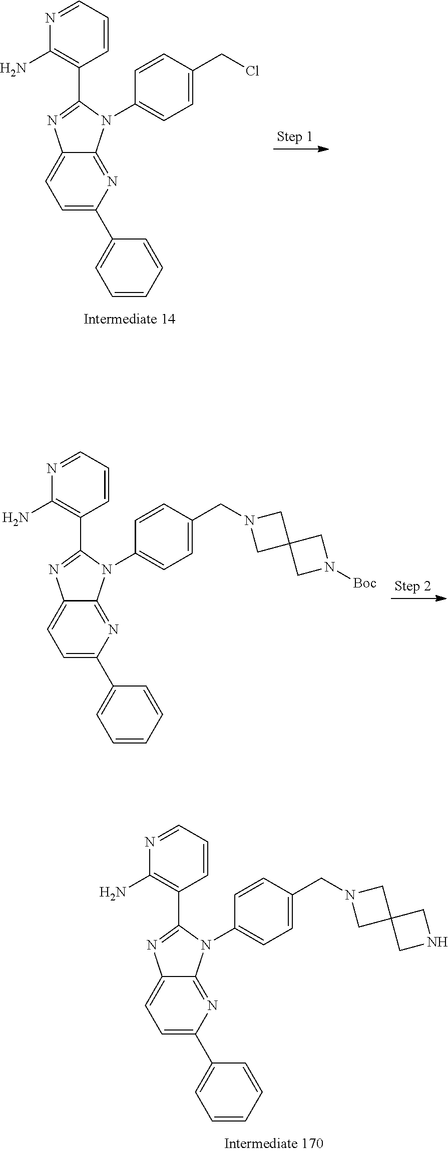 [SMILES: Nc1ncccc1-c1nc2ccc(-c3ccccc3)nc2n1-c1ccc(CCl)cc1 ; CC(C)(C)OC(=O)N1CC2(CN(Cc3ccc(-n4c(-c5cccnc5N)nc5ccc(-c6ccccc6)nc54)cc3)C2)C1 ; Nc1ncccc1-c1nc2ccc(-c3ccccc3)nc2n1-c1ccc(CN2CC3(CNC3)C2)cc1]

### Step 1: tert-Butyl 6-(4-(2-(2-aminopyridin-3-yl)-5-phenyl-3H-imidazo[4,5-b]pyridin-3-yl)benzyl)-2,6-diazaspiro[3.3]heptane-2-carboxylate

To a solution of Intermediate 14 (200 mg, 486 μmol) in MeCN (3 mL) were added K₂CO₃ (268 mg, 1.94 mmol), tert-butyl 2,6-diazaspiro[3.3]heptane-2-carboxylate (106 mg, 534 μmol) and NaI (7.28 mg, 48 μmol) at 25° C. The reaction mixture was stirred at 80° C. for 3 hr. The reaction mixture was filtered and concentrated under reduced pressure. The residue was purified by silica gel flash chromatography (Eluent of 0˜7% MeOH in CH₂Cl₂) to give tert-butyl 6-(4-(2-(2-aminopyridin-3-yl)-5-phenyl-3H-imidazo[4,5-b]pyridin-3-yl)benzyl)-2,6-diazaspiro[3.3]heptane-2-carboxylate (150 mg, yield: 54%) as a yellow solid. MS: m/z=574.6 [M+H]⁺.

### Step 2: 3-(3-(4-((2,6-Diazaspiro[3.3]heptan-2-yl)methyl)phenyl)-5-phenyl-3H-imidazo[4,5-b]pyridin-2-yl)pyridin-2-amine

To a solution of tert-butyl 6-(4-(2-(2-aminopyridin-3-yl)-5-phenyl-3H-imidazo[4,5-b]pyridin-3-yl)benzyl)-2,6-diazaspiro[3.3]heptane-2-carboxylate (150 mg, 261 μmol) in CH₂Cl₂ (3 mL) was added TFA (1.54 g, 13.5 mmol) at 25° C. The reaction mixture was stirred at 25° C. for 2 hr. The reaction mixture was concentrated to dryness to give 3-(3-(4-((2,6-diazaspiro[3.3]heptan-2-yl)methyl)phenyl)-5-phenyl-3H-imidazo[4,5-b]pyridin-2-yl)pyridin-2-amine (Intermediate 170, 120 mg, TFA salt, yield: 81%) as a yellow solid. MS: m/z=474.2 [M+H]⁺. ¹H NMR (400 MHz, Dimethylsulfoxide-d₆) δ 8.26 (d, 0.1=8.4 Hz, 1H), 8.04-7.97 (m, 4H), 7.49-7.44 (m, 2H), 7.42-7.37 (m, 5H), 7.15 (dd, J=7.6, 1.6 Hz, 1H), 6.99 (br s, 2H), 6.39 (dd, J=8.0, 5.2 Hz, 1H), 3.58 (s, 2H), 3.56 (s, 4H), 3.25 (s, 4H), 1.23 (s, 1H).

### Intermediate 171: 2-(4-(2-(2-Aminopyridin-3-yl)-5-phenyl-3H-imidazo[4,5-b]pyridin-3-yl)benzyl)-2-azaspiro[3.3]heptan-6-amine

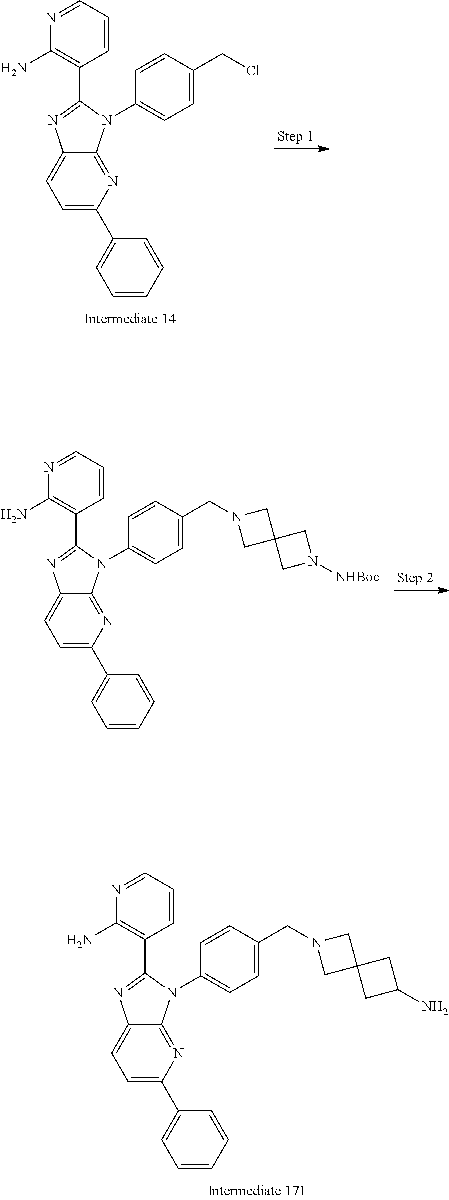 [SMILES: Nc1ncccc1-c1nc2ccc(-c3ccccc3)nc2n1-c1ccc(CCl)cc1 ; CC(C)(C)OC(=O)NN1CC2(CN(Cc3ccc(-n4c(-c5cccnc5N)nc5ccc(-c6ccccc6)nc54)cc3)C2)C1 ; Nc1ncccc1-c1nc2ccc(-c3ccccc3)nc2n1-c1ccc(CN2CC3(CC(N)C3)C2)cc1]

### Step 1: Tert-butyl (2-(4-(2-(2-aminopyridin-3-yl)-5-phenyl-3H-imidazo[4,5-b]pyridin-3-yl)benzyl)-2-azaspiro[3.3]heptan-6-yl)carbamate

To a solution of Intermediate 14 (500 mg, 1.21 mmol) in DMF (8 mL) were added tert-butyl (2-azaspiro[3.3]heptan-6-yl)carbamate (258 mg, 1.21 mmol), NaI (18.2 mg, 121 μmol) and K₂CO₃ (671 mg, 4.86 mmol). The mixture was stirred at 80° C. for 1 hr. The mixture was diluted with H₂O (10 mL) and extracted with CH₂Cl₂ (20 mL×2). The combined organic layers were washed with brine (10 mL×2), dried over Na₂SO₄, filtered, and concentrated under reduced pressure. The residue was purified by silica gel flash chromatography (Eluent of 0˜100% EtOAc in petroleum ether) to give tert-butyl (2-(4-(2-(2-aminopyridin-3-yl)-5-phenyl-3H-imidazo[4,5-b]pyridin-3-yl)benzyl)-2-azaspiro[3.3]heptan-6-yl)carbamate (150 mg, yield: 21%) as a yellow solid. MS: m/z=588.3 [M+H]⁺.

### Step 2: 2-(4-(2-(2-Aminopyridin-3-yl)-5-phenyl-3H-imidazo[4,5-b]pyridin-3-yl)benzyl)-2-azaspiro[3.3]heptan-6-amine

To a solution of tert-butyl (2-(4-(2-(2-aminopyridin-3-yl)-5-phenyl-3H-imidazo[4,5-b]pyridin-3-yl)benzyl)-2-azaspiro[3.3]heptan-6-yl)carbamate (150 mg, 255 μmol) in CH₂Cl₂ (5 mL) was added TFA (87.3 mg, 765 μmol). The mixture was stirred at 25° C. for 0.5 hr. The mixture was diluted with H₂O (10 mL) and pH was adjusted to about 8 with NaHCO₃ (aq). The mixture was extrated with CH₂Cl₂ (10 mL×2). The combined organic layers were washed with brine (10 mL×2), dried over Na₂SO₄, filtered and concentrated under reduced pressure. The residue was purified by prep-TLC (CH₂Cl₂:MeOH=10:1) to give 2-(4-(2-(2-aminopyridin-3-yl)-5-phenyl-3H-imidazo[4,5-b]pyridin-3-yl)benzyl)-2-azaspiro[3.3]heptan-6-amine (Intermediate 171, 123 mg, yield: 83%) as a light-yellow solid. MS: m/z=488.3 [M+H]+. ¹H NMR (400 MHz, Methanol-d₄) 58.20 (d, J=8.8 Hz, 1H), 8.05-8.01 (m, 2H), 7.99 (dd, J=5.2, 1.6 Hz, 1H), 7.95 (d, 1=8.4 Hz, 1H), 7.58-7.50 (m, 2H), 7.43-7.39 (m, 2H), 7.34 (dd, J=7.6, 2.0 Hz, 1H), 6.49 (dd, J=7.6, 4.8 Hz, 1H), 6.21-6.17 (m, 2H), 5.65 (dd, J=7.6, 4.4 Hz, 1H), 4.26-4.18 (m, 1H), 4.00 (s, 2H), 3.77 (s, 2H), 3.66 (s, 2H), 2.65-2.60 (m, 2H), 2.22-2.17 (m, 2H).

### Intermediate 172: 3-(3-(4-((4-Aminopiperidin-1-yl)methyl)phenyl)-5-(3-fluorophenyl)-3H-imidazo[4,5-b]pyridin-2-yl)pyridin-2-amine

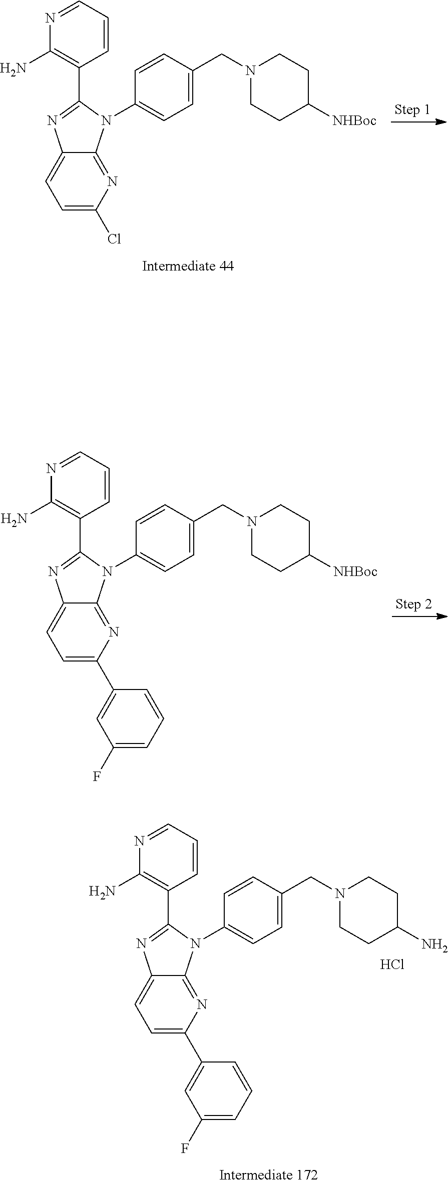 [SMILES: CC(C)(C)OC(=O)NC1CCN(Cc2ccc(-n3c(-c4cccnc4N)nc4ccc(Cl)nc43)cc2)CC1 ; CC(C)(C)OC(=O)NC1CCN(Cc2ccc(-n3c(-c4cccnc4N)nc4ccc(-c5cccc(F)c5)nc43)cc2)CC1 ; Nc1ncccc1-c1nc2ccc(-c3cccc(F)c3)nc2n1-c1ccc(CN2CCC(N)CC2)cc1 ; Cl]

### Step 1: tert-Butyl (1-(4-(2-(2-aminopyridin-3-yl)-5-(3-fluorophenyl)-3H-imidazo[4,5-b]pyridin-3-yl)benzyl)piperidin-4-yl)carbamate

A mixture of Intermediate 44 (200 mg, 375 μmol), (3-fluorophenyl)boronic acid (79 mg, 562 μmol), Pd(dppf)Cl₂ (27 mg, 37 μmol), Cs₂CO₃ (244 mg, 749 μmol) in 1,4-dioxane (3 mL) and H₂O (0.3 mL) was degassed and purged with N₂ three times, then the mixture was stirred at 100° C. for 16 hr under N₂ atmosphere. The reaction mixture was diluted with H₂O (20 mL) and extracted with CH₂Cl₂ (30 mL×2). The combined organic layers were dried over Na₂SO₄, filtered and concentrated under reduced pressure. After purified by silica gel flash chromatography (Eluent of 0˜7% MeOH in CH₂Cl₂), tert-butyl (1-(4-(2-(2-aminopyridin-3-yl)-5-(3-fluorophenyl)-3H-imidazo[4,5-b]pyridin-3-yl)benzyl)piperidin-4-yl)carbamate (138 mg, yield: 48%) was obtained as a brown solid. MS: m/z=594.3 [M+H]⁺.

### Step 2: 3-(3-(4-((4-Aminopiperidin-1-yl)methyl)phenyl)-5-(3-fluorophenyl)-3H-imidazo[4,5-b]pyridin-2-yl)pyridin-2-amine

To a solution of tert-butyl (1-(4-(2-(2-aminopyridin-3-yl)-5-(3-fluorophenyl)-3H-imidazo[4,5-b]pyridin-3-yl)benzyl)piperidin-4-yl)carbamate (135 mg, 227 μmol) in CH₂Cl₂ (3 mL) was added HCl in 1,4-dioxane (4 M, 1 mL). The mixture was stirred at 25° C. for 1 hr. The reaction mixture was concentrated under reduced pressure. The crude product was triturated with MeOH (2 mL) at 25° C. for 30 min. The suspension was filtered, the filter cake washed with 0.5 mL MeOH and concentrated under reduced pressure to give 3-(3-(4-((4-aminopiperidin-1-yl)methyl)phenyl)-5-(3-fluorophenyl)-3H-imidazo[4,5-b]pyridin-2-yl)pyridin-2-amine (Intermediate 172, 15 mg 2HCl salt, yield: 11%) as a light-yellow solid. MS: m/z=494.3 [M+H]⁺. ¹H NMR (400 MHz, Dimethylsulfoxide-d₆) δ 11.38-11.04 (m, 1H), 8.45-8.36 (m, 4H), 8.18-8.10 (m, 2H), 7.94 (d, J=8.0 Hz, 1H), 7.91-7.81 (m, 4H), 7.72-7.64 (m, 2H), 7.57-7.51 (m, 1H), 7.31-7.20 (m, 2H), 7.18-7.12 (m, 1H), 6.94-6.85 (m, 1H), 4.37-4.34 (m, 2H), 3.57 (s, 2H), 3.33-3.30 m, 1H), 3.12-3.02 (m, 2H), 2.22-2.14 (m, 2H), 2.09-1.95 (m, 2H). ¹⁹F NMR (400 MHz, Dimethylsulfoxide-d₆) 6-112.71.

### Intermediate 173: (S)-3-(3-(4-((3-Aminopiperidin-1-yl)methyl)phenyl)-5-phenyl-3H-imidazo[4,5-b]pyridin-2-yl)pyridin-2-amine

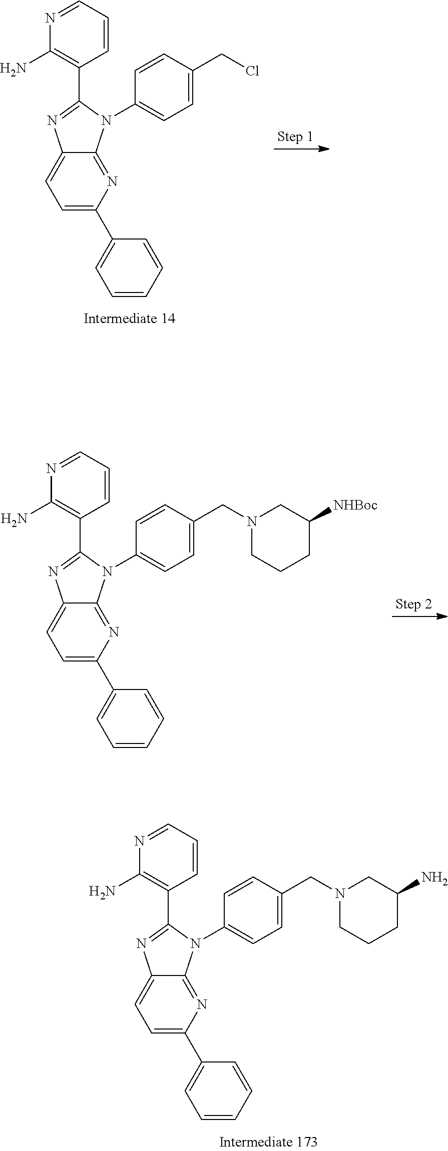 [SMILES: Nc1ncccc1-c1nc2ccc(-c3ccccc3)nc2n1-c1ccc(CCl)cc1 ; CC(C)(C)OC(=O)N[C@H]1CCCN(Cc2ccc(-n3c(-c4cccnc4N)nc4ccc(-c5ccccc5)nc43)cc2)C1 ; Nc1ncccc1-c1nc2ccc(-c3ccccc3)nc2n1-c1ccc(CN2CCC[C@H](N)C2)cc1]

### Step 1: (S)-Tert-butyl (1-(4-(2-(2-aminopyridin-3-yl)-5-phenyl-3H-imidazo[4,5-b]pyridin-3-yl)benzyl)piperidin-3-yl)carbamate

To a solution of Intermediate 14 (800 mg, 1.9 mmol) in ACN (6 mL) were added tert-butyl N-[(3S)-3-piperidyl]carbamate (427 mg, 2.1 mmol), NaI (29.1 mg, 194 μmol) and K₂CO₃ (1.1 g, 7.8 mmol). The mixture was stirred at 80° C. for 2 hr. Then the reaction mixture was concentrated under reduced pressure to give a crude product. The residue was purified by silica gel flash chromatography (Eluent of 0˜50% EtOAc in petroleum ether) to give (S)-tert-butyl (1-(4-(2-(2-aminopyridin-3-yl)-5-phenyl-3H-imidazo[4,5-b]pyridin-3-yl)benzyl)piperidin-3-yl)carbamate (400 mg, 29% yield) as a brown solid. MS: m/z=576.2 [M+H]⁺.

### Step 2: (S)-3-(3-(4-((3-Aminopiperidin-1-yl)methyl)phenyl)-5-phenyl-3H-imidazo[4,5-b]pyridin-2-yl)pyridin-2-amine

To a solution of tert-butyl N-[(3S)-1-[[4-[2-(2-amino-3-pyridyl)-5-phenyl-imidazo[4,5-b]pyridin-3-yl]phenyl]methyl]-3-piperidyl]carbamate (27 mg, 46.9 μmol) in CH₂Cl₂ (1 mL) was added TFA (10.7 mg, 93.8 μmol). The mixture was stirred at 25° C. for 0.5 hr. The mixture was concentrated under reduced pressure to give the crude (26 mg TFA salt, yield: 100%). The crude was purified by prep-HPLC (column: Welch Xtimate C18 150*25 mm 5 μm; mobile phase: [water (HCl)-ACN]; B %: 5%-35%, 8 min) to give (S)-3-(3-(4-((3-aminopiperidin-1-yl)methyl)phenyl)-5-phenyl-3H-imidazo[4,5-b]pyridin-2-yl)pyridin-2-amine (Intermediate 173, 5.3 mg HCl salt) as a light-yellow lyophilized powder. MS: m/z=476.2 [M+H]⁺. 1H NMR (400 MHz, Methanol-d₄) 58.32 (d, J=8.4 Hz, 1H), 8.10-7.98 (m, 4H), 7.94 (d, J=8.4 Hz, 2H), 7.89 (dd, J=7.6, 1.2 Hz, 1H), 7.74 (d, J=8.4 Hz, 2H), 7.49-7.34 (m, 3H), 6.93 (dd, J=7.6, 6.4 Hz, 1H), 4.62 (s, 2H), 3.89-3.56 (m, 3H), 3.28-3.12 (m, 2H), 2.26-1.99 (m, 3H), 1.90-1.66 (m, 1H).

### Intermediate 174: 6-(2-(2-Aminopyridin-3-yl)-3-(4-(chloromethyl)phenyl)-3H-imidazo[4,5-b]pyridin-5-yl)-1-methylpyridin-2(1H)-one

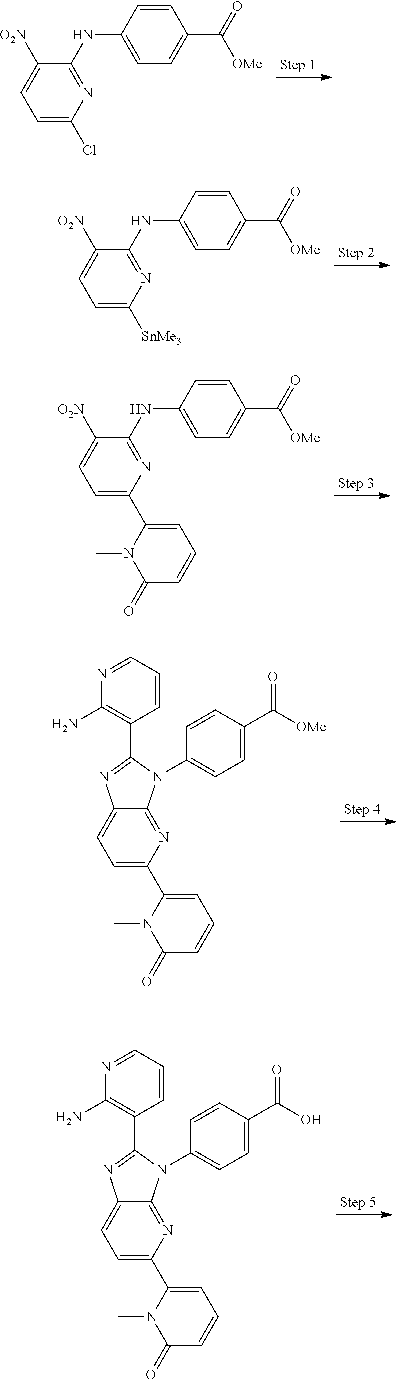

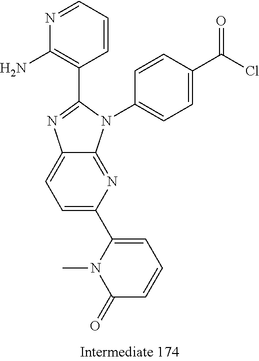 [SMILES: Cn1c(-c2ccc3nc(-c4cccnc4N)n(-c4ccc(C(=O)Cl)cc4)c3n2)cccc1=O]

### Step 1: Methyl 4-((3-nitro-6-(trimethylstannyl)pyridin-2-yl)amino)benzoate

A mixture of methyl 4-((6-chloro-3-nitropyridin-2-yl)amino)benzoate (refer to Intermediate 13 for detail procedures, 5 g, 16.3 mmol), (Me₃Sn)₂ (9.43 g, 28.8 mmol), and Pd(PPh₃)₄ (300 mg, 813 μmol) in 1,4-dioxane (50 mL) was degassed and purged with N₂ three times, and then the mixture was stirred at 90° C. for 4 hr under N₂ atmosphere. The reaction mixture was concentrated under reduced pressure to give methyl 4-((3-nitro-6-(trimethylstannyl)pyridin-2-yl)amino)benzoate (7 g, crude) as a black brown solid, which was directly used in the next step.

### Step 2: Methyl 4-((1′-methyl-5-nitro-6′-oxo-1′,6′-dihydro-[2,2′-bipyridin]-6-yl)amino)benzoate

A mixture of methyl 4-((3-nitro-6-(trimethylstannyl)pyridin-2-yl)amino)benzoate (7 g, 16.1 mmol), 6-bromo-1-methylpyridin-2(1H)-one (3.62 g, 19.3 mmol), and Pd(PPh₃)₄ (296 mg, 803 μmol) in 1,4-dioxane (60 mL) was degassed and purged with N₂ three times, then the mixture was stirred at 100° C. for 16 hr under N₂ atmosphere. The residue was diluted with CH₂Cl₂ (50 mL) and extracted with H₂O (40 mL×3). The combined organic layers were washed with brine (30 mL×2), dried over Na₂SO₄, filtered and concentrated under reduced pressure. After purified by silica gel flash chromatography (Eluent of 20˜25% EtOAc in CH₂Cl₂), methyl 4-((1′-methyl-5-nitro-6′-oxo-1′,6′-dihydro-[2,2′-bipyridin]-6-yl)amino)benzoate (9.4 g, purity: 29%) was obtained as an orange red solid. MS: m/z=381.0 [M+H]⁺.

### Step 3: Methyl 4-(2-(2-aminopyridin-3-yl)-5-(1-methyl-6-oxo-1,6-dihydropyridin-2-yl)-3H-imidazo[4,5-b]pyridin-3-yl)benzoate

To a solution of methyl 4-((1′-methyl-5-nitro-6′-oxo-1′,6′-dihydro-[2,2′-bipyridin]-6-yl)amino)benzoate (225 mg, 1.84 mmol) in DMSO (15 mL) was added Na₂S₂O₄ (1.28 g, 7.36 mmol). The mixture was stirred at 100° C. for 16 hr. The residue was diluted with H₂O (20 mL) and extracted with CH₂Cl₂ (20 mL×3). The combined organic layers were washed with brine (20 mL×2), dried over Na₂SO₄, filtered and concentrated under reduced pressure. After purified by silica gel flash chromatography (Eluent of 2˜3%, MeOH in CH₂Cl₂), methyl 4-(2-(2-aminopyridin-3-yl)-5-(1-methyl-6-oxo-1,6-dihydropyridin-2-yl)-3H-imidazo[4,5-b]pyridin-3-yl)benzoate (300 mg, yield: 31%) was obtained as a black brown solid. MS: m/z=453.0 [M+H]⁺. ¹H NMR (400 MHz, Chlororm-d) δ 8.28-8.23 (m, 3H), 7.94 (dd, J=5.2 Hz, 1.2 Hz, 1H), 7.58 (d, J=8.4 Hz, 1H), 7.50-7.44 (m, 3H), 7.38-7.34 (m, 1H), 7.29 (d, J=7.6 Hz, 1H), 6.67-6.63 (m, 1H), 6.50 (dd, J=7.6, 5.2 Hz, 1H), 6.27 (dd, J=5.6 Hz, 1.2 Hz, 1H), 3.99 (s, 3H), 3.45 (s, 3H).

### Step 4: 6-(2-(2-Aminopyridin-3-yl)-3-(4-(hydroxymethyl)phenyl)-3H-imidazo[4,5-b]pyridin-5-yl)-1-methylpyridin-2(1H)-one

To a solution of methyl 4-(2-(2-aminopyridin-3-yl)-5-(1-methyl-6-oxo-1,6-dihydropyridin-2-yl)-3H-imidazo[4,5-b]pyridin-3-yl)benzoate (100 mg, 221 μmol) in THF (3 mL) was added LiAlH₄ (2.5 M, 177 μL) at 0° C. The mixture was stirred at 2-5° C. for 2 hr. The reaction mixture was quenched by Na₂SO₄·10H₂O (about 0.5 g) at 0° C., and the mixture was filtered. The filter cake was washed by CH₂Cl₂ (5 mL×3), and the filtrate was concentrated under reduced pressure to give 6-(2-(2-aminopyridin-3-yl)-3-(4-(hydroxymethyl)phenyl)-3H-imidazo[4,5-b]pyridin-5-yl)-1-methylpyridin-2(1H)-one (82 mg, yield: 89%) as a yellow solid. Compound 6 was directly used to the next step without purification. MS: m/z=425.3 [M+H]+.

### Step 5: 6-(2-(2-Aminopyridin-3-yl)-3-(4-(chloromethyl)phenyl)-3H-imidazo[4,5-b]pyridin-5-yl)-1-methylpyridin-2(1H)-one

To a solution of 6-(2-(2-aminopyridin-3-yl)-3-(4-(hydroxymethyl)phenyl)-3H-imidazo[4,5-b]pyridin-5-yl)-1-methylpyridin-2(1H)-one (300 mg, 707 μmol) in CH₂Cl₂ (3 mL) was added SOCl₂ (168 mg, 1.41 mmol) at 0° C. The mixture was stirred at 40° C. for 2 hr. The reaction mixture was quenched with H₂O (0.5 mL) at 0° C., then filtered and concentrated under reduced pressure to give 6-(2-(2-aminopyridin-3-yl)-3-(4-(chloromethyl)phenyl)-3H-imidazo[4,5-b]pyridin-5-yl)-1-methylpyridin-2(1H)-one (Intermediate 174, 300 mg, yield: 89%) as a yellow solid. MS: m/z=443.2 [M+H]⁺. ¹H NMR (400 MHz, Methanol-d₄) δ 8.43 (d, J=8.4 Hz, 1H), 8.05-8.01 (m, 1H), 7.92-7.88 (m, 1H), 7.76 (d, J=8.4 Hz, 1H), 7.70-7.62 (m, 3H), 7.53 (d, J=8.0 Hz, 2H), 6.90-6.85 (m, 1H), 6.71-6.66 (m, 1H), 6.62-6.57 (m, 1H), 4.75 (s, 2H), 3.46 (s, 3H).

### Intermediate 175: 4-(Piperidin-4-ylamino)-1,3,5-triazine-2-carbonitrile

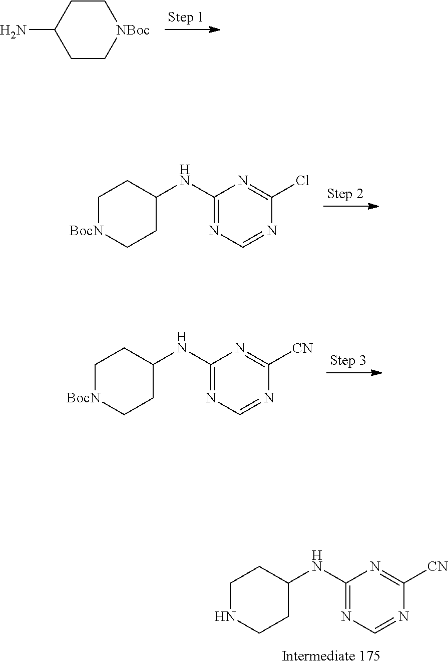 [SMILES: CC(C)(C)OC(=O)N1CCC(N)CC1 ; CC(C)(C)OC(=O)N1CCC(Nc2ncnc(Cl)n2)CC1 ; CC(C)(C)OC(=O)N1CCC(Nc2ncnc(C#N)n2)CC1 ; N#Cc1ncnc(NC2CCNCC2)n1]

### Step 1: tert-Butyl 4-((4-chloro-1,3,5-triazin-2-yl)amino)piperidine-1-carboxylate

To a solution of tert-butyl piperidin-4-ylcarbamate (6 g, 29.9 mmol) in THF (70 mL) was added DIEA (5.81 g, 44.9 mmol). The mixture was stirred at 0° C. for 10 min. Then 2,4-dichloro-1,3,5-triazine (4.49 g, 29.9 mmol) in THF (30 mL) was added to the mixture. The mixture was stirred at 25° C. for 16 hr. The mixture was partitioned between EtOAc (600 mL) and H₂O (200 mL). The separated organic layer was washed with brine (60 mL) and dried over Na₂SO₄, filtered and concentrated. After purified by silica gel flash chromatography (Eluent of 0˜27% EtOAc in petroleum ether), tert-butyl 4-((4-chloro-1,3,5-triazin-2-yl)amino)piperidine-1-carboxylate (5 g, yield: 31%) was obtained as a light-yellow solid. MS: m/z=314.0 [M+H]+.

### Step 2: tert-Butyl 4-((4-cyano-1,3,5-triazin-2-yl)amino)piperidine-1-carboxylate

To a solution of tert-butyl 4-((4-chloro-1,3,5-triazin-2-yl)amino)piperidine-1-carboxylate (5 g, 16 mmol) in DMSO (110 mL) were added KCN (2.01 g, 31 mmol) and DABCO (357 mg, 3.19 mmol). The mixture was stirred at 25° C. for 1 hr. The reaction mixture was poured into H₂O (1 L). The resulting mixture was extracted with EtOAc (500 mL×2). The combined organic layers were washed with brine (100 mL), dried over Na₂SO₄, filtered and concentrated under reduced pressure. After purified by flash chromatography on silica gel (Eluent of 0˜30% EtOAc in petroleum ether), tert-butyl 4-((4-cyano-1,3,5-triazin-2-yl)amino)piperidine-1-carboxylate (1.5 g, yield: 28%) was obtained as a off white solid. MS: m/z=305.0 [M+H]r. ¹H NMR (400 MHz, Chloroform-d) δ 8.55 (d, J=31.2 HZ, 1H), 5.99-5.89 (m, 1H), 4.15-4.00 (m, 3H), 2.95-2.85 (m, 2H), 2.01-1.98 (m, 2H), 1.45 (s, 11H).

### Step 3: 4-(Piperidin-4-ylamino)-1,3,5-triazine-2-carbonitrile

To a solution of tert-butyl 4-((4-cyano-1,3,5-triazin-2-yl)amino)piperidine-1-carboxylate (200 mg, 689 μmol) in CH₂Cl₂ (3 mL) was added TFA (1.02 g, 8.07 mmol). The mixture was stirred at 25° C. for 1 hr. The reaction mixture was concentrated under reduced pressure. The crude 4-(piperidin-4-ylamino)-1,3,5-triazine-2-carbonitrile (Intermediate 175, 209 mg TFA salt, yield: 100%) was used into the next step without further purification.

### Intermediate 176: Methyl 4-(2-(2-aminopyridin-3-yl)-3H-imidazo[4,5-b]pyridin-3-yl)benzoate

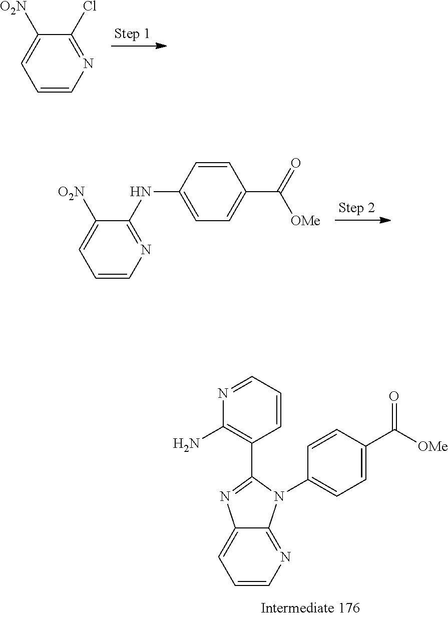 [SMILES: O=[N+]([O-])c1cccnc1Cl ; COC(=O)c1ccc(Nc2ncccc2[N+](=O)[O-])cc1 ; COC(=O)c1ccc(-n2c(-c3cccnc3N)nc3cccnc32)cc1]

### Step 1: Methyl 4-((3-nitropyridin-2-yl)amino)benzoate

To a solution of methyl 4-aminobenzoate (10 g, 66 mmol) were added DIEA (111 g, 861 mmol) and 2-chloro-3-nitropyridine (12.6 g, 79 mmol). The mixture was stirred at 130° C. for 16 hr. The mixture was diluted with H₂O (500 mL) and extracted with EtOAc (500 mL×2). The combined organic layers were washed with brine (1000 mL), dried over Na₂SO₄, filtered and concentrated under reduced pressure to give a residue. After purified by column chromatography (Eluent of 0˜10% EtOAc in petroleum ether), methyl 4-((3-nitropyridin-2-yl)amino)benzoate (7.2 g, yield: 40%) was obtained as a yellow solid. MS: m/z=274.0 [M+H]⁺. ¹H NMR (400 MHz, Chloroform-d) δ 10.32 (br s, 1H), 8.58-8.53 (m, 2H), 8.05 (d, J=8.8 Hz, 2H), 7.81 (d, 0.1=8.8 Hz, 2H), 6.93 (dd, 1=8.0, 4.8 Hz, 1H), 3.91 (s, 3H).

### Step 2: Methyl 4-(2-(2-aminopyridin-3-yl)-3H-imidazo[4,5-b]pyridin-3-yl)benzoate

To a solution of methyl 4-((3-nitropyridin-2-yl)amino)benzoate (7 g, 26 mmol) in DMSO (300 mL) were added Na₂S₂O₄ (18 g, 102 mmol) and 2-aminonicotinaldehyde (3.8 g, 31 mmol). The mixture was stirred at 110° C. for 16 hr. The mixture was diluted with H₂O (1000 mL) and extracted with CH₂Cl₂ (1000 mL×2). The combined organic layers were washed with brine (1000 mL), dried over Na₂SO₄, filtered and concentrated under reduced pressure. After purified by column chromatography (Eluent of 0˜43% EtOAc in petroleum ether), methyl 4-(2-(2-aminopyridin-3-yl)-3H-imidazo[4,5-b]pyridin-3-yl)benzoate (Intermediate 176, 4.1 g, yield: 45%) was obtained as a yellow solid. MS: m/z=346.1 [M+H]⁺. ¹H NMR (400 MHz, Chloroform-d) δ 8.43-8.36 (m, 1H), 8.21 (d, J=8.4 Hz, 2H), 8.14-8.06 (m, 2H), 7.49 (d, J=8.4 Hz, 2H), 7.33 (dd, J=8.0, 4.8 Hz, 1H), 7.04 (dd, J=7.6, 1.2 Hz, 1H), 6.59 (br s, 2H), 6.37 (dd, J=8.0, 4.8 Hz, 1H), 3.96 (s, 3H).

### Intermediate 177: 3-(3-(4-(Chloromethyl-d₂) phenyl)-3H-imidazo[4,5-b]pyridin-2-yl)pyridin-2-amine

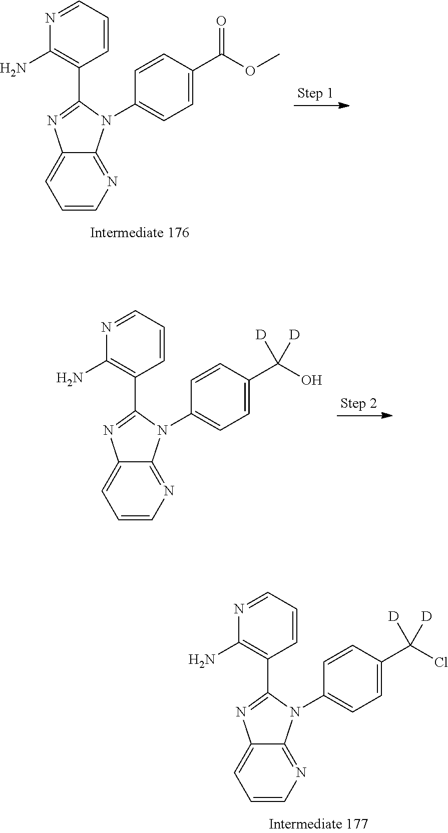 [SMILES: COC(=O)c1ccc(-n2c(-c3cccnc3N)nc3cccnc32)cc1 ; [2H]C([2H])(O)c1ccc(-n2c(-c3cccnc3N)nc3cccnc32)cc1 ; [2H]C([2H])(Cl)c1ccc(-n2c(-c3cccnc3N)nc3cccnc32)cc1]

### Step 1: (4-(2-(2-Aminopyridin-3-yl)-3H-imidazo[4,5-b]pyridin-3-yl)phenyl)methan-d₂-ol

To a solution of Intermediate 176 (400 mg, 1.2 mmol) in THF (10 mL) was added LiAlD₄ (88 mg, 2.3 mmol) at 0° C. The resulting mixture was stirred at 25° C. for 1 hr. The reaction mixture was quenched by Na₂SO₄·10H₂O (2 g) at 0° C., filtered and concentrated under reduced pressure to give a (4-(2-(2-Aminopyridin-3-yl)-3H-imidazo[4,5-b]pyridin-3-yl)phenyl)methan-d₂-ol (370 mg) as a yellow solid, which was used for next step directly. MS: m/z=320.0 [M+H]⁺.

### Step 2: 3-(3-(4-(Chloromethyl-d₂) phenyl)-3H-imidazo[4,5-b]pyridin-2-yl)pyridin-2-amine

To a solution of 4-(2-(2-aminopyridin-3-yl)-3H-imidazo[4,5-b]pyridin-3-yl)phenyl)methan-d₂-ol (370 mg, 1.2 mmol) in CH₂Cl₂ (5 mL) was added dropwise SOCl₂ (1.6 g, 14 mmol). The resulting mixture was stirred at 40° C. for 1 hr. The reaction mixture was quenched by Na₂CO₃ (5 mL) at 25° C., diluted with H₂O (10 mL) and extracted with EtOAc (10 mL×2). The combined organic layers were washed with brine (10 mL), dried over Na₂SO₄, filtered and concentrated under reduced pressure. After purified by flash chromatography on silica gel (Eluent of 0˜50% EtOAc in petroleum ether), 3-(3-(4-(chloromethyl-d₂) phenyl)-3H-imidazo[4,5-b]pyridin-2-yl)pyridin-2-amine (Intermediate 177, 90 mg, yield: 23% for 2 steps) was obtained as a yellow solid. MS: m/z=337.9 [M+H]⁺.

### Intermediate 178: 4-(2-(2-Aminopyridin-3-yl)-5-chloro-3H-imidazo[4,5-b]pyridin-3-yl)benzyl acetate

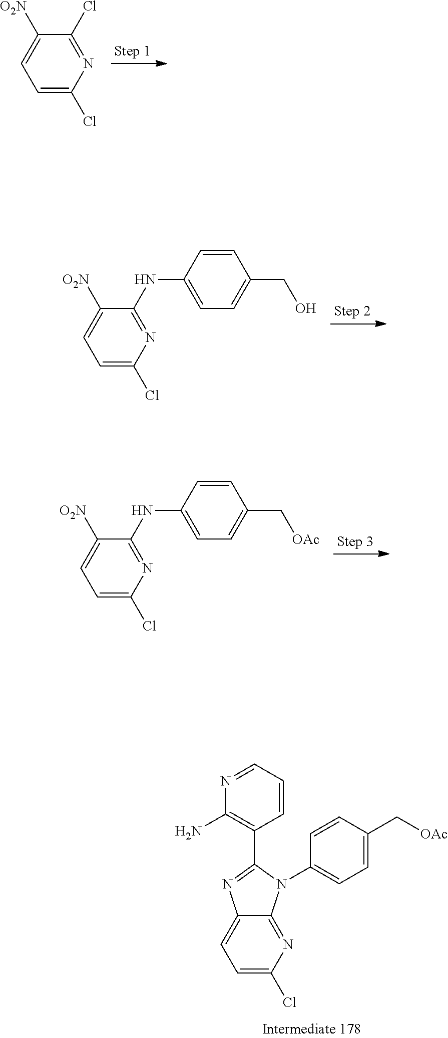 [SMILES: O=[N+]([O-])c1ccc(Cl)nc1Cl ; O=[N+]([O-])c1ccc(Cl)nc1Nc1ccc(CO)cc1 ; CC(=O)OCc1ccc(Nc2nc(Cl)ccc2[N+](=O)[O-])cc1 ; CC(=O)OCc1ccc(-n2c(-c3cccnc3N)nc3ccc(Cl)nc32)cc1]

### Step 1: (4-((6-Chloro-3-nitropyridin-2-yl)amino)phenyl)methanol

To a solution of 2,6-dichloro-3-nitropyridine (50 g, 259 mmol) and (4-aminophenyl)methanol (32 g, 259 mmol) in 1,4-dioxane (500 mL) was added DIEA (100 g, 777 mmol). The mixture was stirred at 70° C. for 1 hr. The reaction mixture was diluted with H₂O (500 mL) and extracted with CH₂Cl2 (300 mL×2). The combined organic layers were washed with brine (200 mL), dried over Na₂SO₄, filtered and concentrated to give (4-((6-chloro-3-nitropyridin-2-yl)amino)phenyl)methanol (67 g, yield: 92%) as an orange solid, which was used in the next step without purification. ¹H NMR (400 MHz, Dimethylsulfoxide-d₆) δ 10.11 (br s, 1H), 8.53 (d, J=8.4 Hz, 1H), 7.53 (d, J=8.4 Hz, 2H), 7.33 (d, J=8.4 Hz, 2H), 6.98 (d, J=8.8 Hz, 1H), 5.20 (br s, 1H), 4.49 (s, 2H).

### Step 2: 4-((6-Chloro-3-nitropyridin-2-yl)amino)benzyl acetate

To a solution of (4-((6-chloro-3-nitropyridin-2-yl)amino)phenyl)methanol (67 g, 239 mmol) in CH₂Cl₂ (600 mL) was added Et₃N (72.7 g, 718 mmol) and DMAP (2.93 g, 23.9 mmol) at 0° C. The mixture was stirred at 0° C. for 10 min, and then Ac₂O (24.5 g, 239 mmol) in CH₂Cl₂ (50 mL) was added dropwise at 0° C. The resulting mixture was stirred at 25° C. for 16 hr. The reaction mixture was diluted with H₂O (500 mL) and extracted with CH₂Cl₂ (500 mL×2). The combined organic layers were washed with brine (500 mL), dried over Na₂SO₄, filtered and concentrated under reduced pressure. After purified by silica gel flash chromatography (Eluent of 0˜1% MeOH in CH₂Cl₂), 4-((6-chloro-3-nitropyridin-2-yl)amino)benzyl acetate (36 g, yield: 45%) was obtained as an orange solid. MS: m/z=321.8 [M+H]⁺. ¹H NMR (400 MHz, Dimethylsulfoxide-d₆) δ 10.14 (s, 1H), 8.55 (d, J=8.4 Hz, 1H), 7.61-7.58 (m, 2H), 7.39-7.37 (m, 1H), 7.08-6.97 (m, 2H), 5.07 (s, 2H), 2.07 (s, 3H).

### Step 3: 4-(2-(2-Aminopyridin-3-yl)-5-chloro-3H-imidazo[4,5-b]pyridin-3-yl)benzyl acetate

To a solution of 4-((6-chloro-3-nitropyridin-2-yl)amino)benzyl acetate (24 g, 74.6 mmol) and 2-aminonicotinaldehyde (10 g, 82 mmol) in DMSO (600 mL) was added Na₂S₂O₄ (52 g, 298 mmol). The mixture was stirred at 100° C. for 16 hr. The reaction mixture was diluted with H₂O (1500 mL) and extracted with CH₂Cl₂ (150 mL×3). The combined organic layers were washed with brine (500 mL), dried over Na₂SO₄, filtered and concentrated under reduced pressure to give a residue. After purified by column chromatography (Eluent of 0˜50% EtOAc in petroleum ether), 4-(2-(2-aminopyridin-3-yl)-5-chloro-3H-imidazo[4,5-b]pyridin-3-yl)benzyl acetate (Intermediate 178, 5 g, yield: 14%) was obtained as a black brown solid. MS: m/z=394.0 [M+H]⁺. ¹H NMR (400 MHz, Chloroform-d) δ 8.10-7.99 (m, 2H), 7.52 (d, J=8.0 Hz, 2H), 7.41-7.32 (m, 3H), 7.08 (d, J=7.2 Hz, 1H), 6.80 (br s, 2H), 6.40 (dd, J=7.6, 5.2 Hz, 1H), 5.20 (s, 2H), 2.16 (s, 3H).

### Intermediate 179: 4-((7-Azaspiro[3.5]nonan-2-yl)amino)pyrimidine-2-carbonitrile

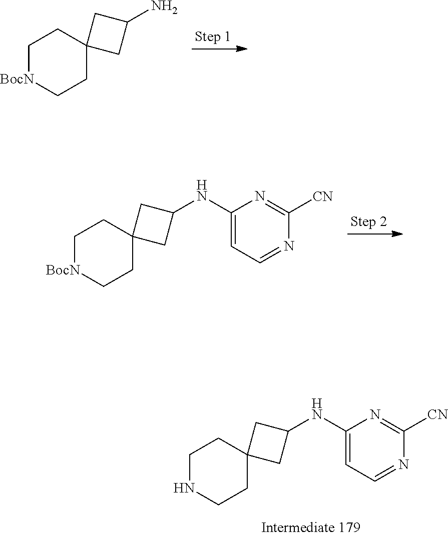 [SMILES: CC(C)(C)OC(=O)N1CCC2(CC1)CC(N)C2 ; CC(C)(C)OC(=O)N1CCC2(CC1)CC(Nc1ccnc(C#N)n1)C2 ; N#Cc1nccc(NC2CC3(CCNCC3)C2)n1]

### Step 1: Tert-butyl 2-((2-cyanopyrimidin-4-yl)amino)-7-azaspiro[3.5]nonane-7-carboxylate

To a solution of tert-butyl 2-amino-7-azaspiro[3.5]nonane-7-carboxylate (500 mg, 2.08 mmol) and 4-chloropyrimidine-2-carbonitrile (290 mg, 2.08 mmol) in ACN (5 mL) were added K₂CO₃ (864 mg, 6.24 mmol) and NaI (62.4 mg, 416 μmol). The mixture was stirred at 80° C. for 2 hr. The reaction mixture was poured into H₂O (20 mL), extracted with EtOAc (30 mL×3). The organic phase was dried over anhydrous Na₂SO₄, filtered, and concentrated under reduced pressure. After purified by silica gel flash chromatography (Eluent of 0%˜50% EtOAc in petroleum ether), tert-butyl 2-((2-cyanopyrimidin-4-yl)amino)-7-azaspiro[3.5]nonane-7-carboxylate (505 mg, yield: 70%) was obtained as an off white solid. MS: m/z=344.0 [M+H]⁺. ¹H NMR (400 MHz, Dimethylsulfoxide-d₆) δ 8.47-8.22 (m, 1H), 8.09 (d, J=6.0 Hz, 1H), 6.65 (d, J=6.0 Hz, 1H), 4.60-4.12 (m, 1H), 3.98-3.86 (m, 2H), 3.83-3.72 (m, 2H), 2.61-2.54 (m, 2H), 2.14-2.04 (m, 2H), 1.39 (s, 9H).

### Step 2: 4-((7-Azaspiro[3.5]nonan-2-yl)amino)pyrimidine-2-carbonitrile

To a solution of tert-butyl 2-((2-cyanopyrimidin-4-yl)amino)-7-azaspiro[3.5]nonane-7-carboxylate (240 mg, 699 μmol) in CH₂Cl₂ (3 mL) was added TFA (13.5 mmol, 1 mL). The mixture was stirred at 25° C. for 1 hr. The mixture was concentrated under reduced pressure to give 4-((7-azaspiro[3.5]nonan-2-yl)amino)pyrimidine-2-carbonitrile (Intermediate 179, 230 mg, TFA salt, crude) as a yellow oil. MS: m/z=244.0 [M+H]⁺

### Intermediate 180: 4-((2-Azaspiro[3.3]heptan-6-yl)amino)pyrimidine-2-carbonitrile

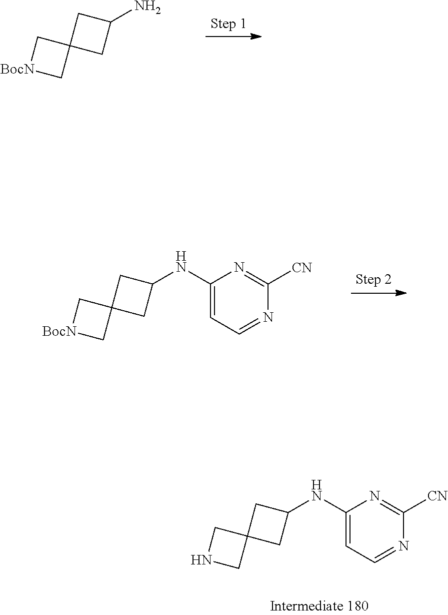 [SMILES: CC(C)(C)OC(=O)N1CC2(CC(N)C2)C1 ; CC(C)(C)OC(=O)N1CC2(CC(Nc3ccnc(C#N)n3)C2)C1 ; N#Cc1nccc(NC2CC3(CNC3)C2)n1]

### Step 1: Tert-butyl 6-((2-cyanopyrimidin-4-yl)amino)-2-azaspiro[3.3]heptane-2-carboxylate

To a solution of tert-butyl 6-amino-2-azaspiro[3.3]heptane-2-carboxylate (1 g, 4.71 mmol) and 4-chloropyrimidine-2-carbonitrile (657 mg, 4.71 mmol) in ACN (10 mL) were added K₂CO₃ (1.95 g, 14.1 mmol) and NaI (141 mg, 942 μmol). The mixture was stirred at 80° C. for 1 hr. The reaction mixture was poured into H₂O (20 mL), extracted with EtOAc (30 mL×3). The organic phase was dried over anhydrous Na₂SO₄, filtered and concentrated under reduced pressure. After purified by silica gel flash chromatography (Eluent of 0%˜50% EtOAc in petroleum ether), tert-butyl 6-((2-cyanopyrimidin-4-yl)amino)-2-azaspiro[3.3]heptane-2-carboxylate (1.6 g, yield: 73%) was obtained as a yellow solid. MS: m/z=316.1 [M+H]⁺. ¹H NMR (400 MHz, Dimethylsulfoxide-d₆) δ 8.43-8.21 (m, 1H), 8.09 (d, J=5.6 Hz, 1H), 6.63 (d, J=5.6 Hz, 1H), 4.33-4.14 (m, 1H), 3.91 (s, 2H), 3.78 (s, 2H), 2.60-2.52 (m, 2H), 2.16-2.03 (m, 2H), 1.36 (s, 9H).

### Step 2: 4-((2-Azaspiro[3.3]heptan-6-yl)amino)pyrimidine-2-carbonitrile

To a solution of tert-butyl 6-((2-cyanopyrimidin-4-yl)amino)-2-azaspiro[3.3]heptane-2-carboxylate (200 mg, 634 μmol) in CH₂Cl₂ (3 mL) was added TFA (13.5 mmol, 1 mL). The mixture was stirred at 25° C. for 1 hr. The mixture was concentrated under reduced pressure to give 4-((2-azaspiro[3.3]heptan-6-yl)amino)pyrimidine-2-carbonitrile (Intermediate 180, 195 mg, TFA salt, crude) as a yellow oil.

### Intermediate 181: 4-(Azetidin-3-ylamino)pyrimidine-2-carbonitrile

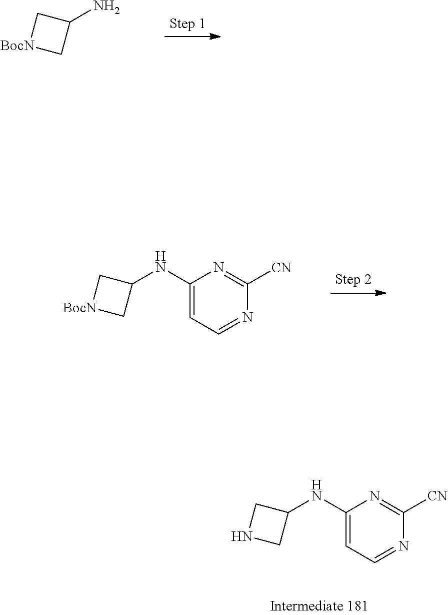 [SMILES: CC(C)(C)OC(=O)N1CC(N)C1 ; CC(C)(C)OC(=O)N1CC(Nc2ccnc(C#N)n2)C1 ; N#Cc1nccc(NC2CNC2)n1]

### Step 1: Tert-butyl 3-((2-cyanopyrimidin-4-yl)amino)azetidine-1-carboxylate

To a solution of tert-butyl 3-aminoazetidine-1-carboxylate (1 g, 5.81 mmol) and 4-chloropyrimidine-2-carbonitrile (810 mg, 5.81 mmol) in ACN (10 mL) were added NaI (174 mg, 1.16 mmol) and K₂CO₃ (2.41 g, 17.4 mmol). The mixture was stirred at 80° C. for 2 hr. The reaction mixture was diluted with H₂O (20 mL) and extracted with EtOAc (30 mL×3). The combined organic layers were washed with brine (30 mL), dried over Na₂SO₄, filtered and concentrated under reduced pressure. After purified by silica gel flash chromatography (Eluent of 0˜50% EtOAc in petroleum ether), tert-butyl 3-((2-cyanopyrimidin-4-yl)amino)azetidine-1-carboxylate (1.4 g, yield: 86%) was obtained as a white solid. MS: m/z=276.0 [M+H]⁺. ¹H NMR (400 MHz, Dimethylsulfoxide-d₆) δ 8.69 (d, J=6.4 Hz, 1H), 8.18 (d, J=5.2 Hz, 1H), 6.73 (d, J=6.0 Hz, 1H), 4.58 (s, 1H), 4.20-4.15 (m, 2H), 3.76-3.69 (m, 2H), 1.39 (s, 9H).

### Step 2: 4-(Azetidin-3-ylamino)pyrimidine-2-carbonitrile

To a solution of tert-butyl 3-((2-cyanopyrimidin-4-yl)amino)azetidine-1-carboxylate (200 mg, 726 μmol) in CH₂Cl₂ (5 mL) was added TFA (13.5 mmol, 1 mL). The mixture was stirred at 25° C. for 1 hr. The mixture was concentrated under reduced pressure to give 4-(azetidin-3-ylamino)pyrimidine-2-carbonitrile (Intermediate 181, 205 mg, TFA salt, crude) as a yellow oil.

### Intermediate 182: 4-((2-azaspiro[3.5]nonan-7-yl)amino)pyrimidine-2-carbonitrile

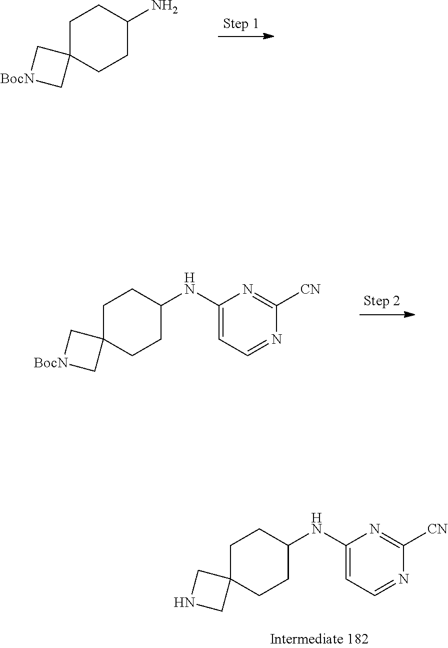 [SMILES: CC(C)(C)OC(=O)N1CC2(CCC(N)CC2)C1 ; CC(C)(C)OC(=O)N1CC2(CCC(Nc3ccnc(C#N)n3)CC2)C1 ; N#Cc1nccc(NC2CCC3(CC2)CNC3)n1]

### Step 1: Tert-butyl 7-((2-cyanopyrimidin-4-yl)amino)-2-azaspiro[3.5]nonane-2-carboxylate

To a solution of tert-butyl 7-amino-2-azaspiro[3.5]nonane-2-carboxylate (1 g, 4.16 mmol) and 4-chloropyrimidine-2-carbonitrile (580 mg, 4.16 mmol) in ACN (10 mL) were added NaI (125 mg, 832 μmol) and K₂CO₃ (1.73 g, 12.5 mmol). The mixture was stirred at 80° C. for 2 hr. The reaction mixture was diluted with H₂O (20 mL) and extracted with EtOAc (30 mL×3). The combined organic layers were washed with brine (30 mL), dried over Na₂SO₄, filtered and concentrated under reduced pressure. After purified by silica gel flash chromatography (Eluent of 0˜30% EtOAc in petroleum ether), tert-butyl 7-((2-cyanopyrimidin-4-yl)amino)-2-azaspiro[3.5]nonane-2-carboxylate (1.2 g, yield: 76%) was obtained as a white solid. MS: m/z=344.0 [M+H]⁺. ¹H NMR (400 MHz, Chloroform-d) δ 8.1 (s, 1H), 6.41 (d, J=6.0 Hz, 1H), 5.50-4.98 (m, 1H), 3.62 (d, J=8.4 Hz, 4H), 3.48 (s, 2H), 2.01-1.90 (m, 4H), 1.67-1.58 (m, 2H), 1.44 (s, 9H), 1.31-1.22 (m, 2H).

### Step 2: 4-((2-Azaspiro[3.5]nonan-7-yl)amino)pyrimidine-2-carbonitrile

To a solution of tert-butyl 7-((2-cyanopyrimidin-4-yl)amino)-2-azaspiro[3.5]nonane-2-carboxylate (200 mg, 582 μmol) in CH₂Cl₂ (10 mL) was added TFA (13.5 mmol, 1 mL). The mixture was stirred at 25° C. for 0.5 hr. The mixture was stirred at 25° C. for 0.5 hr. The mixture was concentrated under reduced pressure to give 4-((2-azaspiro[3.5]nonan-7-yl)amino)pyrimidine-2-carbonitrile (Intermediate 182, 205 mg, TFA salt, crude) as a yellow oil.

### Intermediate: 183: 4-(2,6-diazaspiro[3.3]heptan-2-yl)pyrimidine-2-carbonitrile

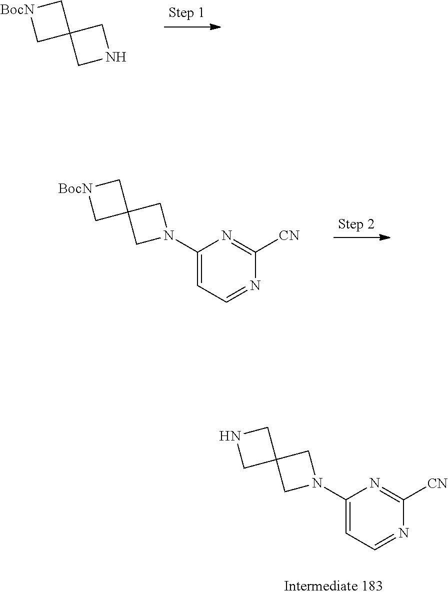 [SMILES: CC(C)(C)OC(=O)N1CC2(CNC2)C1 ; CC(C)(C)OC(=O)N1CC2(C1)CN(c1ccnc(C#N)n1)C2 ; N#Cc1nccc(N2CC3(CNC3)C2)n1]

### Step 1: Tert-butyl 6-(2-cyanopyrimidin-4-yl)-2,6-diazaspiro[3.3]heptane-2-carboxylate

To a solution of tert-butyl 2,6-diazaspiro[3.3]heptane-2-carboxylate (180 mg, 908 μmol) and 4-chloropyrimidine-2-carbonitrile (127 mg, 908 μmol) in ACN (10 mL) were added NaI (27.2 mg, 182 μmol) and K₂CO₃ (376 mg, 2.76 mmol). The mixture was stirred at 80° C. for 2 hr. The reaction mixture was diluted with H₂O (20 mL) and extracted with EtOAc (30 mL×3). The combined organic layers were washed with brine (30 mL), dried over Na₂SO₄, filtered and concentrated under reduced pressure. After purified by silica gel flash chromatography (Eluent of 0˜48% EtOAc in petroleum ether), tert-butyl 6-(2-cyanopyrimidin-4-yl)-2,6-diazaspiro[3.3]heptane-2-carboxylate (216 mg, yield: 78%) was obtained as an off white solid. MS: m/z=301.9 [M+H]⁺. ¹H NMR (400 MHz, Dimethylsulfoxide-d₆) δ 8.22 (d, J=6.0 Hz, 1H), 6.63 (d, J=6.0 Hz, 1H), 4.23 (s, 3H), 4.03 (s, 3H), 3.31 (s, 2H), 1.37 (s, 9H).

### Step 2: 4-(2,6-Diazaspiro[3.3]heptan-2-yl)pyrimidine-2-carbonitrile

To a solution of tert-butyl 6-(2-cyanopyrimidin-4-yl)-2,6-diazaspiro[3.3]heptane-2-carboxylate (180 mg, 597 μmol) in CH₂Cl₂ (5 mL) was added TFA (13.5 mmol, 1 mL). The mixture was stirred at 25° C. for 1 hr. The mixture was concentrated under reduced pressure to give 4-(2,6-diazaspiro[3.3]heptan-2-yl)pyrimidine-2-carbonitrile (Intermediate 183, 201 mg, TFA salt, crude) as a light yellow oil.

### Intermediate 184: 4-(3,8-Diazabicyclo[3.2.1]octan-3-yl)pyrimidine-2-carbonitrile

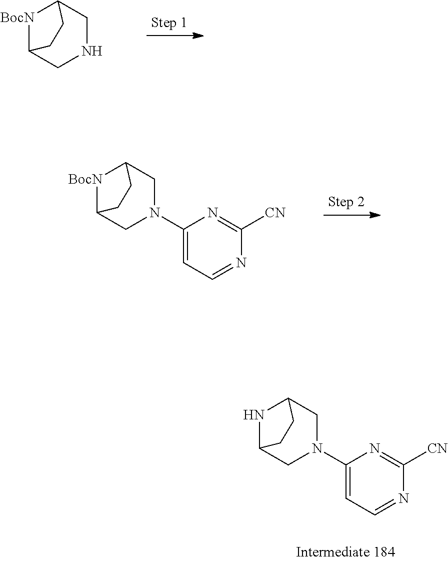 [SMILES: CC(C)(C)OC(=O)N1C2CCC1CNC2 ; CC(C)(C)OC(=O)N1C2CCC1CN(c1ccnc(C#N)n1)C2 ; N#Cc1nccc(N2CC3CCC(C2)N3)n1]

### Step 1: Tert-butyl 3-(2-cyanopyrimidin-4-yl)-3,8-diazabicyclo[3.2.1]octane-8-carboxylate

To a solution of tert-butyl 3,8-diazabicyclo[3.2.1]octane-8-carboxylate (500 mg, 2.36 mmol), 4-chloropyrimidine-2-carbonitrile (329 mg, 2.36 mmol) in ACN (5 mL) were added K₂CO₃ (977 mg, 7.07 mmol) and NaI (70.6 mg, 471 μmol). The mixture was stirred at 80° C. for 1 hr. The reaction mixture was poured into H₂O (10 mL), extracted with EtOAc (15 mL×3). After purified by silica gel flash chromatography (Eluent of 0%˜50% EtOAc in petroleum ether), tert-butyl 3-(2-cyanopyrimidin-4-yl)-3,8-diazabicyclo[3.2.1]octane-8-carboxylate (505 mg, yield: 70%) was obtained as an off white solid. MS: m/z=316.0 [M+H]⁺. ¹H NMR (400 MHz, Dimethylsulfoxide-d₆) δ 8.30 (d, J=6.4 Hz, 1H), 7.06 (d, J=6.4 Hz, 1H), 4.40-4.15 (m, 3H), 3.98-3.70 (m, 1H), 3.18-2.90 (m, 2H), 1.90-1.75 (m, 2H), 1.61-1.54 (m, 2H), 1.43 (s, 9H).

### Step 2: 4-(3,8-Diazabicyclo[3.2.1]octan-3-yl)pyrimidine-2-carbonitrile

To a solution of tert-butyl 3-(2-cyanopyrimidin-4-yl)-3,8-diazabicyclo[3.2.1]octane-8-carboxylate (200 mg, 634 μmol) in CH₂Cl₂ (2 mL) was added TFA (13.5 mmol, 1 mL). The mixture was stirred at 25° C. for 1 hr. The mixture was stirred at 25° C. for 1 hr. The mixture was concentrated under reduced pressure to give 4-(3,8-diazabicyclo[3.2.]octan-3-yl)pyrimidine-2-carbonitrile (Intermediate 184, 210 mg, TFA salt, crude) as a yellow oil. MS: m/z=216.0 [M+H]⁺.

### Intermediate 185: 4-((3aR,6aS)-hexahydropyrrolo[3,4-c]pyrrol-2(1H)-yl)pyrimidine-2-carbonitrile

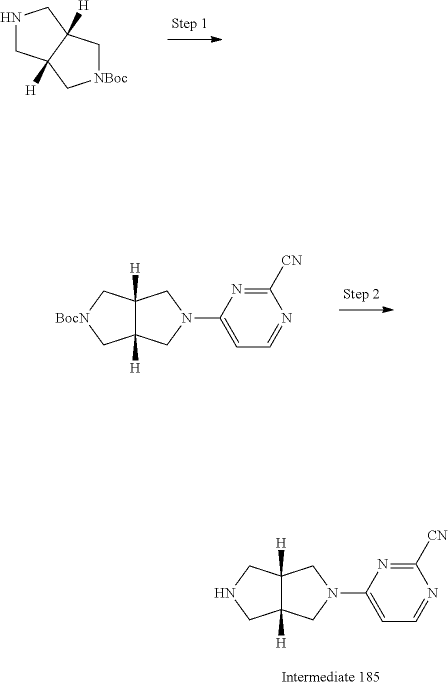 [SMILES: CC(C)(C)OC(=O)N1C[C@H]2CNC[C@H]2C1 ; CC(C)(C)OC(=O)N1C[C@@H]2CN(c3ccnc(C#N)n3)C[C@@H]2C1 ; N#Cc1nccc(N2C[C@H]3CNC[C@H]3C2)n1]

### Step 1: Tert-butyl (3aR,6aS)-5-(2-cyanopyrimidin-4-yl)hexahydropyrrolo[3,4-c]pyrrole-2(1H)-carboxylate

To a solution of tert-butyl (3aR,6aS)-hexahydropyrrolo[3,4-c]pyrrole-2(1H)-carboxylate (500 mg, 2.36 mmol), 4-chloropyrimidine-2-carbonitrile (329 mg, 2.36 mmol) in ACN (5 mL) were added K₂CO₃ (977 mg, 7.07 mmol) and NaI (70.6 mg, 471 μmol). The mixture was stirred at 80° C. for 2 hr. The reaction mixture was poured into H₂O (10 mL), extracted with EtOAc (15 mL×3). After purified by silica gel flash chromatography (Eluent of 0%˜50% EtOAc in petroleum ether), tert-butyl (3aR,6aS)-5-(2-cyanopyrimidin-4-yl)hexahydropyrrolo[3,4-c]pyrrole-2(1H)-carboxylate (610 mg, yield: 82%) was obtained as an off white solid. MS: m/z=316.1 [M+H]⁺. ¹H NMR (400 MHz, Dimethylsulfoxide-d₆) δ 8.24 (d, J=6.0 Hz, 1H), 6.74 (d, J=6.4 Hz, 1H), 3.80-3.68 (m, 1H), 3.66-3.58 (m, 1H), 3.56-3.48 (m, 2H), 3.42-3.35 (m, 2H), 3.17 (dd, J=11.2, 4.4 Hz, 2H), 3.08-2.90 (m, 2H), 1.39 (s, 9H).

### Step 2: 4-((3aR,6aS)-Hexahydropyrrolo[3,4-c]pyrrol-2(1H)-yl)pyrimidine-2-carbonitrile

To a solution of tert-butyl (3aR,6aS)-5-(2-cyanopyrimidin-4-yl)hexahydropyrrolo[3,4-c]pyrrole-2(1H)-carboxylate (200 mg, 634 μmol) in CH₂Cl₂ (3 mL) was added TFA (634 μmol, 47 μL). The mixture was stirred at 25° C. for 0.5 hr. The mixture was concentrated under reduced pressure to give 4-((3aR,6aS)-hexahydropyrrolo[3,4-c]pyrrol-2 (1H)-yl)pyrimidine-2-carbonitrile (Intermediate 185, 210 mg, TFA salt, crude) as a yellow oil. MS: m/z=216.1 [M+H]⁺.

### Intermediate 186: 4-((Piperidin-4-yl-4-d)amino)pyrimidine-2-carbonitrile

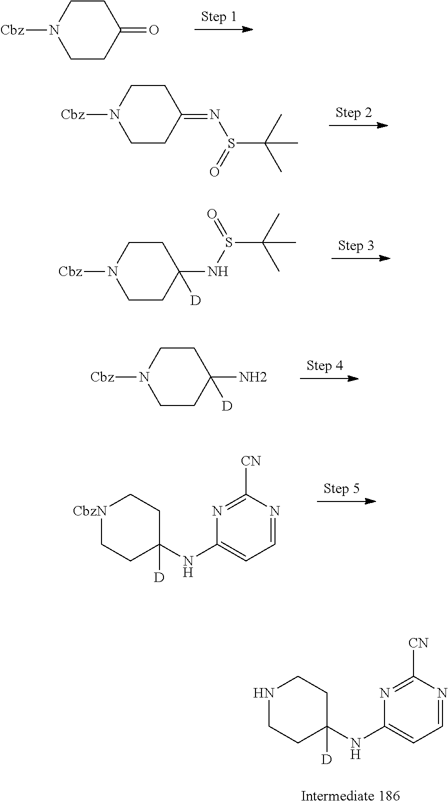

### Step 1. Benzyl 4-((tert-butylsulfinyl)imino)piperidine-1-carboxylate

A mixture of benzyl 4-oxopiperidine-1-carboxylate (5 g, 21.4 mmol), 2-methylpropane-2-sulfinamide (2.86 g, 23.6 mmol), tetraethoxytitanium (8.31 g, 36.4 mmol) in THF (100 mL) was degassed and purged with N₂ three times, and then was stirred at 70° C. for 1 hr under N₂ atmosphere. The reaction mixture was concentrated under reduced pressure. After purified by silica gel flash chromatography (Eluent of 0˜30% EtOAc in petroleum ether), benzyl 4-((tert-butylsulfinyl)imino)piperidine-1-carboxylate (4.3 g, yield: 60%) was obtained as a light-yellow oil. MS: m/z=337.0 [M+H]⁺. ¹H NMR (400 MHz, Chloroform-d) δ 7.38-7.35 (m, 5H), 5.17 (s, 1H), 5.15 (s, 1H), 3.83-3.73 (m, 4H), 3.16-3.11 (m, 1H), 2.90-2.79 (m, 1H), 2.55-2.51 (m, 1H), 2.46-2.43 (m, 1H), 1.23 (s, 9H).

### Step 2: Benzyl 4-((tert-butylsulfinyl)amino)piperidine-1-carboxylate-4-d

To a solution of benzyl 4-((tert-butylsulfinyl)imino)piperidine-1-carboxylate (3 g, 8.92 mmol) in THF (40 mL) was added LiAlD₄ (675 mg, 17.8 mmol) at 0° C. The mixture was stirred at 25° C. for 1 hr. The mixture was diluted with H₂O (60 mL) and extracted with EtOAc (40 mL×3). The combined organic layers were washed with brine (30 mL×3), dried over Na₂SO₄, filtered, and concentrated under reduced pressure. After purified by silica gel flash chromatography (Eluent of 50˜60% EtOAc in CH₂Cl₂), benzyl 4-((tert-butylsulfinyl)amino)piperidine-1-carboxylate-4-d (1.4 g, yield: 39%) was obtained as a colorless oil. MS: m/z=340.1 [M+H]⁺. ¹H NMR (400 MHz, Chloroform-d) δ 7.41-7.30 (m, 5H), 5.13 (s, 2H), 4.14-4.06 (m, 2H), 3.05-2.90 (m, 2H), 2.01-1.93 (m, 2H), 1.52-1.40 (m, 2H), 1.21 (s, 9H).

### Step 3: Benzyl 4-aminopiperidine-1-carboxylate-4-d

To a solution of benzyl 4-((tert-butylsulfinyl)amino)piperidine-1-carboxylate-4-d (1.31 g, 3.86 mmol) in 1,4-dioxane (10 mL) was added HCl/1,4-dioxane (4 M, 1.93 mL). The mixture was stirred at 25° C. for 1 hr. The reaction mixture was filtered and concentrated under reduced pressure to give benzyl 4-aminopiperidine-1-carboxylate-4-d (900 mg, yield: 99%) as a colorless oil. MS: m/z=236.1 [M+H]⁺. ¹H NMR (400 MHz, Methanol-d₄) δ 7.41-7.28 (m, 5H), 5.12 (s, 2H), 4.25-4.21 (m, 2H), 3.37-3.33 (m, 2H), 2.02 (d, J=12.8 Hz, 2H), 1.56-1.45 (m, 2H).

### Step 4: Benzyl 4-((2-cyanopyrimidin-4-yl)amino)piperidine-1-carboxylate-4-d

To a solution of benzyl 4-aminopiperidine-1-carboxylate-4-d (500 mg, 2.12 mmol), 4-chloropyrimidine-2-carbonitrile (297 mg, 2.12 mmol) in DMF (10 mL) were added NaI (63.7 mg, 425 μmol) and K₂CO₃ (881 mg, 6.37 mmol). The mixture was stirred at 50° C. for 1 hr. The mixture was diluted with H₂O (10 mL) and extracted with CH₂Cl₂ (20 mL×3). The combined organic layers were washed with brine (20 mL×3), dried over Na₂SO₄, filtered and concentrated under reduced pressure. After purified by silica gel flash chromatography (Eluent of 40˜50% EtOAc in petroleum ether), benzyl 4-((2-cyanopyrimidin-4-yl)amino)piperidine-1-carboxylate-4-d (330 mg, yield: 45%) was obtained as a colorless oil. MS: m/z=339.1 [M+H]⁺. ¹H NMR (400 MHz, Chloroform-d) δ 8.26-8.12 (m, 1H), 7.39-7.34 (m, 5H), 6.44 (d, J=5.6 Hz, 1H), 5.15 (s, 2H), 4.30-4.08 (m, 2H), 3.11-2.95 (m, 2H), 2.09-2.00 (m, 2H), 1.66 (br s, 2H).

### Step 5: 4-((Piperidin-4-yl-4-d)amino)pyrimidine-2-carbonitrile

To a solution of benzyl 4-((2-cyanopyrimidin-4-yl)amino)piperidine-1-carboxylate-4-d (300 mg, 887 μmol) in CH₂Cl₂ (10 mL) was added TMSI (887 mg, 4.43 mmol). The mixture was stirred at 25° C. for 1 hr. The mixture was filtered to give 4-((piperidin-4-yl-4-d)amino)pyrimidine-2-carbonitrile (Intermediate 186, 330 mg, yield: 45%) as a yellow solid. MS: m/z=205.0 [M+H]⁺. ¹H NMR (400 MHz, Dimethylsulfoxide-d₆) δ 8.59-8.41 (m, 1H), 8.21-8.09 (m, 2H), 6.77-6.63 (m, 1H), 3.35-3.27 (m, 2H), 3.13-3.03 (m, 2H), 2.08-2.00 (m, 2H), 1.64-1.56 (m, 2H).

### Intermediate 187: N-(3-(2-(2-Aminopyridin-3-yl)-3-(4-(chloromethyl)phenyl)-3H-imidazo[4,5-b]pyridin-5-yl)phenyl)acetamide

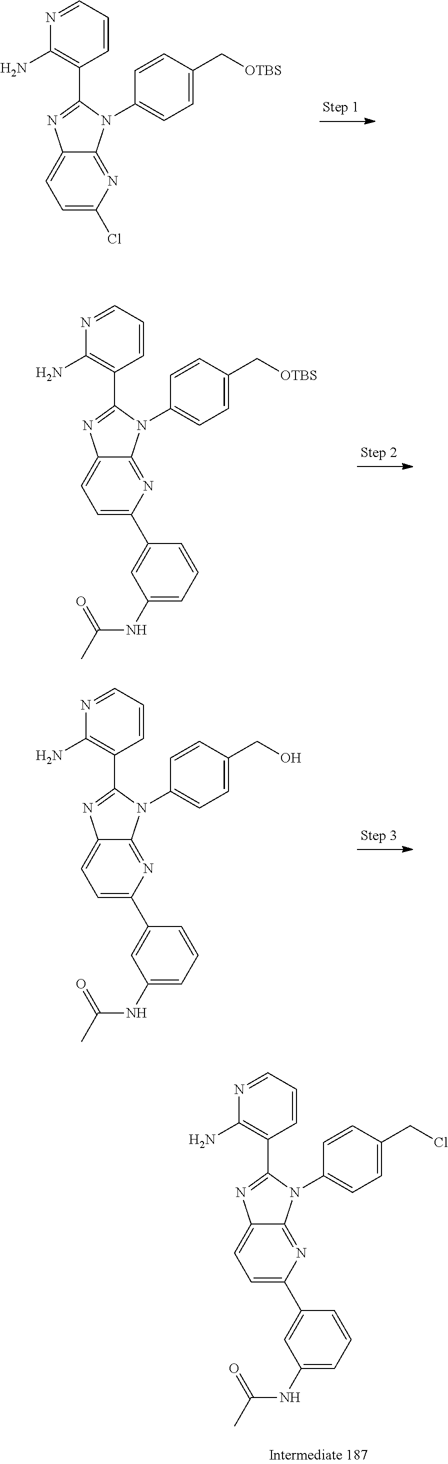

### Step 1: N-(3-(2-(2-Aminopyridin-3-yl)-3-(4-(((tert-butyldimethylsilyl)oxy)methyl)phenyl)-3H-imidazo[4,5-b]pyridin-5-yl)phenyl)acetamide

A mixture of 3-(3-(4-(((tert-butyldimethylsilyl)oxy)methyl)phenyl)-5-chloro-3H-imidazo[4,5-b]pyridin-2-yl)pyridin-2-amine (refer to Intermediate 117 for detail procedures, 500 mg, 1.07 mmol), (3-acetamidophenyl)boronic acid (288 mg, 1.61 mmol), Pd(dppf)Cl₂ (78.5 mg, 107 μmol), K₂CO₃ (444 mg, 3.22 mmol) in 1,4-dioxane (10 mL) and H₂O (2 mL) was degassed and purged with N₂ three times. The mixture was stirred at 100° C. for 16 hr under N₂ atmosphere. The reaction mixture was diluted with H₂O (50 mL) and extracted with CH₂Cl₂ (50 mL×3). The combined organic layers were washed with brine (50 mL), dried over Na₂SO₄, filtered and concentrated under reduced pressure. After purified by silica gel flash chromatography (eluent of 0˜50/a EtOAc in petroleum ether), N-(3-(2-(2-aminopyridin-3-yl)-3-(4-(((tert-butyldimethylsilyl)oxy)methyl)phenyl)-3H-imidazo[4,5-b]pyridin-5-yl)phenyl)acetamide (500 mg, yield: 81%) was obtained as a gray solid. MS: m/z=565.2 [M+H]⁺. ¹H NMR (400 MHz, Dimethylsulfoxide-d₆) δ 10.05 (s, 1H), 8.28 (d, J=8.4 Hz, 1H), 8.12 (s, 1H), 8.01 (dd, J=4.8, 1.6 Hz, 1H), 7.86 (d, J=8.4 Hz, 1H), 7.67 (t, J=8.8 Hz, 2H), 7.54-7.42 (m, 4H), 7.37 (t, J=8.0 Hz, 1H), 7.19 (dd, J=7.6, 1.6 Hz, 1H), 6.99 (br s, 2H), 6.39 (dd, J=7.6, 4.8 Hz, 1H), 4.82 (s, 2H), 2.05 (s, 3H), 0.93 (s, 9H), 0.11 (s, 6H).

### Step 2: N-(3-(2-(2-Aminopyridin-3-yl)-3-(4-(hydroxymethyl)phenyl)-3H-imidazo[4,5-b]pyridin-5-yl)phenyl)acetamide

A mixture of N-(3-(2-(2-aminopyridin-3-yl)-3-(4-(((tert-butyldimethylsilyl)oxy)methyl)phenyl)-3H-imidazo[4,5-b]pyridin-5-yl)phenyl)acetamide (500 mg, 885 μmol) in TBAF (1 M, 5 mL) was stirred at 25° C. for 1 hr. The reaction mixture was diluted with H₂O (50 mL) and extracted with EtOAc (50 mL×3). The combined organic layers were washed with brine (50 mL), dried over Na₂SO₄, filtered, and concentrated under reduced pressure. N-(3-(2-(2-aminopyridin-3-yl)-3-(4-(hydroxymethyl)phenyl)-3H-imidazo[4,5-b]pyridin-5-yl)phenyl)acetamide (400 mg, yield: 89%) was obtained as a gray solid. MS: m/z=451.3 [M+H]⁺. ¹H NMR (400 MHz, Dimethylsulfoxide-d₆) δ 10.17 (s, 1H), 8.38 (d, J=8.4 Hz, 2H), 8.19 (s, 1H), 8.14 (dd, J=6.0, 1.2 Hz, 2H), 7.96-7.90 (m, 3H), 7.70-7.60 (m, 5H), 7.40 (t, J=7.6 Hz, 2H), 6.91 (d, J=7.6, 6.4 Hz, 1H), 4.87 (s, 2H), 2.06 (s, 3H).

### Step 3. N-(3-(2-(2-Aminopyridin-3-yl)-3-(4-(chloromethyl)phenyl)-3H-imidazo[4,5-b]pyridin-5-yl)phenyl)acetamide

To a mixture of N-(3-(2-(2-aminopyridin-3-yl)-3-(4-(hydroxymethyl)phenyl)-3H-imidazo[4,5-b]pyridin-5-yl)phenyl)acetamide (200 mg, 443 μmol) in CH₂Cl₂ (4 mL) was added SOCl₂ (3.28 g, 27.5 mmol). The mixture was stirred at 40° C. for 1 hr. The reaction mixture was filtered, and the filter cake was concentrated under reduced pressure. N-(3-(2-(2-Aminopyridin-3-yl)-3-(4-(chloromethyl)phenyl)-3H-imidazo[4,5-b]pyridin-5-yl)phenyl)acetamide (Intermediate 187, 220 mg, yield: 86%) was obtained as a gray solid. MS: m/z=469.0 [M+H]⁺.

### Intermediate 188: 3-(3-(4-(Chloromethyl)phenyl)-5-vinyl-3H-imidazo[4,5-b]pyridin-2-yl)pyridin-2-amine

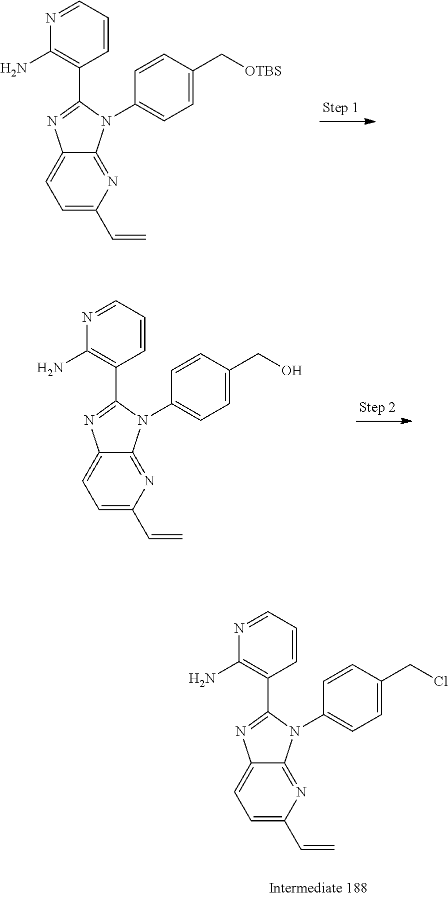 [SMILES: C=Cc1ccc2nc(-c3cccnc3N)n(-c3ccc(CO[Si](C)(C)C(C)(C)C)cc3)c2n1 ; C=Cc1ccc2nc(-c3cccnc3N)n(-c3ccc(CO)cc3)c2n1 ; C=Cc1ccc2nc(-c3cccnc3N)n(-c3ccc(CCl)cc3)c2n1]

### Step 1: (4-(2-(2-Aminopyridin-3-yl)-5-vinyl-3H-imidazo[4,5-b]pyridin-3-yl)phenyl)methanol

To a solution of 3-(3-(4-(((tert-butyldimethylsilyl)oxy)methyl)phenyl)-5-vinyl-3H-imidazo[4,5-b]pyridin-2-yl)pyridin-2-amine (refer to Intermediate 124 for detail procedures, 320 mg, 699 μmol) in THF (4 mL) was added TBAF (1 M, 2.80 mL). The mixture was stirred at 25° C. for 0.5 hr. The reaction mixture was diluted with water (10 mL) and extracted with EtOAc (2×20 mL). The combined organic layers were dried over Na₂SO₄, filtered, and concentrated under reduced pressure. (4-(2-(2-Aminopyridin-3-yl)-5-vinyl-3H-imidazo[4,5-b]pyridin-3-yl)phenyl)methanol (240 mg, crude) was obtained as a brown oil. MS: m/z=344.1 [M+H]⁺.

### Step 2: 3-(3-(4-(Chloromethyl)phenyl)-5-vinyl-3H-imidazo[4,5-b]pyridin-2-yl)pyridin-2-amine

To a solution of (4-(2-(2-aminopyridin-3-yl)-5-vinyl-3H-imidazo[4,5-b]pyridin-3-yl)phenyl)methanol (240 mg, 698 μmol) in CH₂Cl₂ (2 mL) was added SOCl₂ (332 mg, 2.80 mmol). The mixture was stirred at 40° C. for 0.5 hr. The mixture was concentrated under reduced pressure to give 3-(3-(4-(chloromethyl)phenyl)-5-vinyl-3H-imidazo[4,5-b]pyridin-2-yl)pyridin-2-amine (Intermediate 188, 240 mg, yield: 86%, HCl salt) as a yellow solid. MS: m/z=361.9 [M+H]⁺.

### Intermediate 189: 3-(3-(4-(Chloromethyl)phenyl)-5-(5-fluoropyridin-2-yl)-3H-imidazo[4,5-b]pyridin-2-yl)pyridin-2-amine

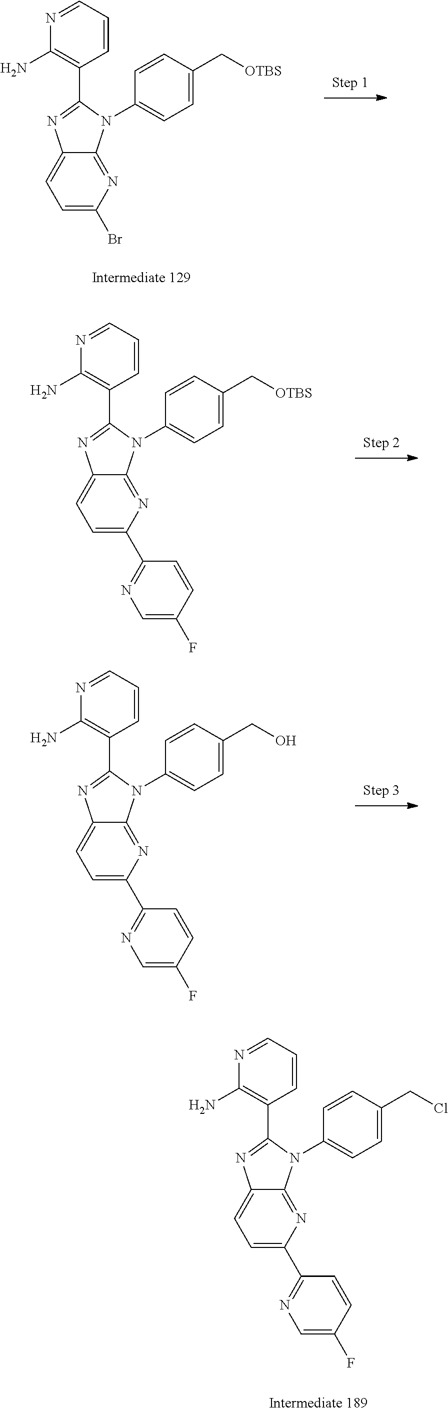 [SMILES: CC(C)(C)[Si](C)(C)OCc1ccc(-n2c(-c3cccnc3N)nc3ccc(Br)nc32)cc1 ; CC(C)(C)[Si](C)(C)OCc1ccc(-n2c(-c3cccnc3N)nc3ccc(-c4ccc(F)cn4)nc32)cc1 ; Nc1ncccc1-c1nc2ccc(-c3ccc(F)cn3)nc2n1-c1ccc(CO)cc1 ; Nc1ncccc1-c1nc2ccc(-c3ccc(F)cn3)nc2n1-c1ccc(CCl)cc1]

### Step 1: 3-(3-(4-(((Tert-butyldimethylsilyl)oxy)methyl)phenyl)-5-(5-fluoropyridin-2-yl)-3H-imidazo[4,5-b]pyridin-2-yl)pyridin-2-amine

To a solution of Intermediate 129 (500 mg, 979 μmol) in 1,4-dioxane (16.5 mL) were added CuBr (105 mg, 734 μmol), Cs₂CO₃ (105 mg, 957 μmol), (5-fluoropyridin-2-yl)boronic acid (690 mg, 957 mmol) and cataCXium A Pd G3 (71 mg, 98 μmol) at 25° C. The reaction mixture was degassed and purged with N₂ three times. The mixture was stirred at 100° C. for 4 hr under N₂. The reaction mixture was then poured into H₂O (50 mL) and extracted with CH₂Cl₂ (20 mL×2). The combined organic layers were washed with brine (40 mL), dried over anhydrous Na₂SO₄, filtered and concentrated. After purified by silica gel flash chromatography (Eluent of 0˜40% EtOAc in petroleum ether), 3-(3-(4-(((tert-butyldimethylsilyl)oxy)methyl)phenyl)-5-(5-fluoropyridin-2-yl)-3H-imidazo[4,5-b]pyridin-2-yl)pyridin-2-amine (450 mg, yield: 85%) was obtained as a brown solid. MS: m/z=527.2 [M+H]⁺. ¹H NMR (400 MHz, Dimethylsulfoxide-d₆) δ 8.67 (d, J=3.2 Hz, 1H), 8.40 (d, J=8.4 Hz, 1H), 8.32 (d, J=8.0 Hz, 1H), 8.25-8.17 (m, 1H), 8.04-7.98 (m, 1H), 7.87-7.78 (m, 1H), 7.55-7.45 (m, 4H), 7.25-7.16 (m, 1H), 7.11-6.88 (m, 2H), 6.43-6.35 (m, 1H), 4.83 (s, 2H), 0.95-0.92 (m, 9H), 0.13-0.11 (m, 6H).

### Step 2: (4-(2-(2-Aminopyridin-3-yl)-5-(5-fluoropyridin-2-yl)-3H-imidazo[4,5-b]pyridin-3-yl)phenyl)methanol

To a solution of 3-(3-(4-(((tert-butyldimethylsilyl)oxy)methyl)phenyl)-5-(5-fluoropyridin-2-yl)-3H-imidazo[4,5-b]pyridin-2-yl)pyridin-2-amine (450 mg, 854 μmol) in THF (5 mL) was added TBAF (670 mg, 2.5 ml, 1 M) at 25° C. The mixture was stirred at 25° C. for 1 hr. The reaction mixture was poured into H₂O (20 mL). The mixture was extracted with CH₂Cl₂ (20 mL×2). The combined organic layers were washed with brine (40 mL), dried over anhydrous Na₂SO₄, filtered and concentrated. (4-(2-(2-Aminopyridin-3-yl)-5-(5-fluoropyridin-2-yl)-3H-imidazo[4,5-b]pyridin-3-yl)phenyl)methanol (300 mg, crude) was obtained as a yellow solid. MS: m/z=413.1 [M+H]⁺. ¹H NMR (400 MHz, Dimethylsulfoxide-d₆) δ 8.66 (d, J=2.4 Hz, 1H), 8.40 (d, J=6.4 Hz, 1H), 8.31 (d, J=8.4 Hz, 1H), 8.22 (dd, J=8.8, 4.8 Hz, 1H), 8.01 (dd, J=4.8, 2.0 Hz, 1H), 7.87-7.80 (m, 1H), 7.53-7.44 (m, 4H), 7.23 (dd, J=7.6, 1.6 Hz, 1H), 6.98 (br s, 2H), 6.42 (dd, J=7.6, 4.8 Hz, 1H), 5.36 (br s, 1H), 4.61 (s, 2H).

### Step 3: 3-(3-(4-(Chloromethyl)phenyl)-5-(5-fluoropyridin-2-yl)-3H-imidazo[4,5-b]pyridin-2-yl)pyridin-2-amine

To a solution of (4-(2-(2-aminopyridin-3-yl)-5-(5-fluoropyridin-2-yl)-3H-imidazo[4,5-b]pyridin-3-yl)phenyl)methanol (300 mg, 727 μmol) in CH₂Cl₂ (5 mL) was added SOCl₂ (433 mg, 3.6 mmol) at 25° C. The mixture was stirred at 40° C. for 0.5 hr. The reaction mixture was concentrated under reduced pressure to give 3-(3-(4-(chloromethyl)phenyl)-5-(5-fluoropyridin-2-yl)-3H-imidazo[4,5-b]pyridin-2-yl)pyridin-2-amine (Intermediate 189, 300 mg, crude) as a yellow solid, which was used in the next step without further purification. MS: m/z=431.0 [M]+. 1H NMR (400 MHz, Dimethylsulfoxide-d₆) δ 8.67 (d, J=2.8 Hz, 1H), 8.41 (d, J=8.4 Hz, 1H), 8.30 (d, J=8.4 Hz, 1H), 8.24 (dd, J=8.8, 4.8 Hz, 1H), 8.01 (dd, J=4.8, 1.6 Hz, 1H), 7.87-7.82 (m, 1H), 7.63 (d, J=8.4 Hz, 2H), 7.53 (d, J=8.4 Hz, 2H), 7.25-7.21 (m, 1H), 6.90 (br s, 2H), 6.44 (dd, J=7.6, 4.8 Hz, 1H), 4.88 (s, 2H). 19F NMR (400 MHz, Dimethylsulfoxide-d₆) 5-127.227

### Intermediate 190: 2-(2-Aminopyridin-3-yl)-3-(4-(chloromethyl)phenyl)-N-methyl-3H-imidazo[4,5-b]pyridin-5-amine

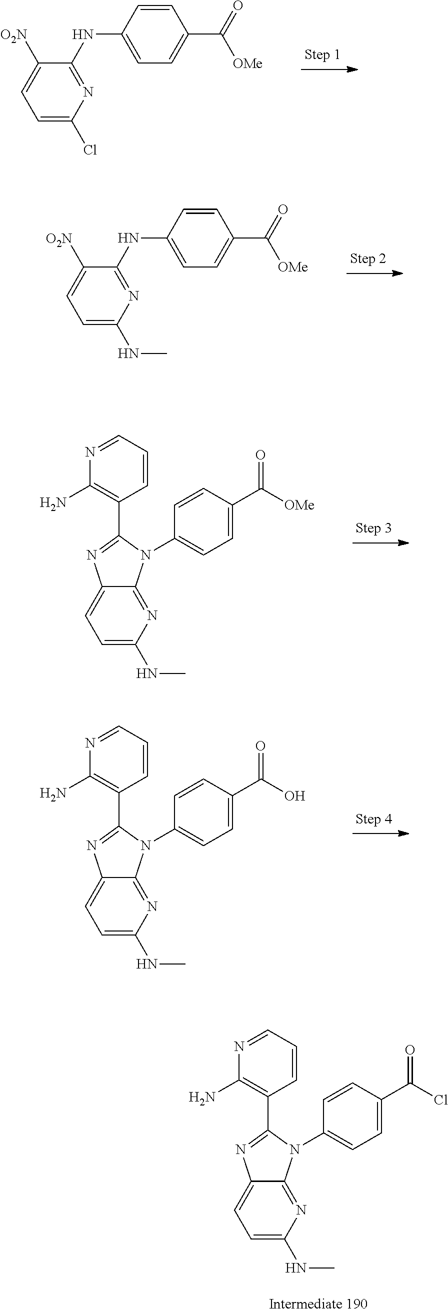 [SMILES: COC(=O)c1ccc(Nc2nc(Cl)ccc2[N+](=O)[O-])cc1 ; CNc1ccc([N+](=O)[O-])c(Nc2ccc(C(=O)OC)cc2)n1 ; CNc1ccc2nc(-c3cccnc3N)n(-c3ccc(C(=O)OC)cc3)c2n1 ; CNc1ccc2nc(-c3cccnc3N)n(-c3ccc(C(=O)O)cc3)c2n1 ; CNc1ccc2nc(-c3cccnc3N)n(-c3ccc(C(=O)Cl)cc3)c2n1]

### Step 1: Methyl 4-((6-(methylamino)-3-nitropyridin-2-yl)amino)benzoate

To a mixture of methyl 4-((6-chloro-3-nitropyridin-2-yl)amino)benzoate (refer to Intermediate 13 for detail procedures, 3 g, 9.75 mmol) in CH₃CN (50 mL) were added MeNH₂·HCl (790 mg, 11.7 mmol) and DIEA (3.78 g, 29.2 mmol). The mixture was stirred at 90° C. for 1 hr. The reaction mixture was filtered and concentrated under reduced pressure. The crude product was triturated with CH₃CN (50 mL) at 25° C. for 5 min to give methyl 4-((6-(methylamino)-3-nitropyridin-2-yl)amino)benzoate (2.6 g, yield: 82%) as a yellow solid. MS: m/z=303.0 [M+H]⁺. ¹H NMR (400 MHz, Dimethylsulfoxide-d₆) δ 11.06 (s, 1H), 8.39 (d, J=4.4 Hz, 1H), 8.11 (d, J=9.6 Hz, 1H), 8.00-7.93 (m, 4H), 6.18 (d, J=9.2 Hz, 1H), 3.83 (s, 3H), 2.94 (d, J=4.4 Hz, 3H).

### Step 2: Methyl 4-(2-(2-aminopyridin-3-yl)-5-(methylamino)-3H-imidazo[4,5-b]pyridin-3-yl)benzoate

To a mixture of methyl 4-((6-(methylamino)-3-nitropyridin-2-yl)amino)benzoate (2.6 g, 8.60 mmol) and 2-aminonicotinaldehyde (1.26 g, 10.3 mmol) in DMSO (40 mL) was added Na₂S₂O₄ (5.99 g, 34.4 mmol). The mixture was stirred at 100° C. for 24 hr. The reaction mixture was diluted with H₂O (100 mL) and extracted with EtOAc (100 mL×3). The combined organic layers were washed with H₂O (100 mL×3), dried over Na₂SO₄, filtered and concentrated under reduced pressure. After purified by silica gel flash chromatography (eluent of 0-50% Ethyl acetate in petroleum ether), methyl 4-(2-(2-aminopyridin-3-yl)-5-(methylamino)-3H-imidazo[4,5-b]pyridin-3-yl)benzoate (1.5 g, yield: 43%) was obtained as a yellow solid. MS: m/z=375.0 [M+H]+. ¹H NMR (400 MHz, Dimethylsulfoxide-d₆) δ 8.06 (d, J=8.4 Hz, 2H), 7.93 (d, J=3.6 Hz, 1H), 7.81 (d, J=8.8 Hz, 1H), 7.52 (d, J=8.4 Hz, 2H), 7.00 (d, J=7.2 Hz, 1H), 6.91 (br s, 2H), 6.75 (d, J=4.4 Hz, 1H), 6.50 (d, J=9.2 Hz, 1H), 6.36 (dd, J=7.2, 4.8 Hz, 1H), 3.88 (s, 3H), 2.72 (d, J=4.4 Hz, 3H).

### Step 3: (4-(2-(2-Aminopyridin-3-yl)-5-((methyl-d₃)amino)-3H-imidazo[4,5-b]pyridin-3-yl)phenyl)methanol

To a solution of methyl 4-(2-(2-aminopyridin-3-yl)-5-(methylamino)-3H-imidazo[4,5-b]pyridin-3-yl)benzoate (1.5 g, 4.01 mmol) in THF (100 mL) was added LiAlH₄ (2.5 M, 2.40 mL) at 0° C. The mixture was stirred at 25° C. for 1 hr. The reaction mixture was quenched with Na₂SO₄·10H₂O (2.5 g) at 0° C., filtered and concentrated under reduced pressure to give (4-(2-(2-aminopyridin-3-yl)-5-(methylamino)-3H-imidazo[4,5-b]pyridin-3-yl)phenyl)methanol (1.2 g, yield: 73%) as a yellow solid. MS: m/z=347.0 [M+H]⁺. ¹H NMR (400 MHz, Dimethylsulfoxide-d₆) δ 7.90 (dd, J=4.8, 1.6 Hz, 1H), 7.79 (d, J=8.8 Hz, 1H), 7.43 (d, J=8.4 Hz, 2H), 7.30 (d, J=8.4 Hz, 2H), 7.06-6.95 (m, 3H), 6.72-6.65 (m, 1H), 6.47 (d, J=8.8 Hz, 1H), 6.31 (dd, J=8.0, 4.8 Hz, 1H), 5.33 (t, J=6.0 Hz, J H), 4.57 (d, J=6.0 Hz, 2H), 2.70 (d, J=4.8 Hz, 3H).

### Step 4: 2-(2-Aminopyridin-3-yl)-3-(4-(chloromethyl)phenyl)-N-methyl-3H-imidazo[4,5-b]pyridin-5-amine

To a solution of (4-(2-(2-aminopyridin-3-yl)-5-(methylamino)-3H-imidazo[4,5-b]pyridin-3-yl)phenyl)methanol (1.2 g, 3.46 mmol) in CH₂Cl₂ (8 mL) was added SOCl₂ (4.91 g, 41.3 mmol). The mixture was stirred at 40° C. for 1 hr. The reaction mixture was filtered, and the filter cake was concentrated under reduced pressure. 2-(2-Aminopyridin-3-yl)-3-(4-(chloromethyl)phenyl)-N-methyl-3H-imidazo[4,5-b]pyridin-5-amine (Intermediate 190, 1.3 g, yield: 95%) was obtained as a yellow solid. MS: m/z=364.9 [M+H]⁺.

### Intermediate 191: 4-(2,7-Diazaspiro[3.5]nonan-2-yl)pyrimidine-2-carbonitrile

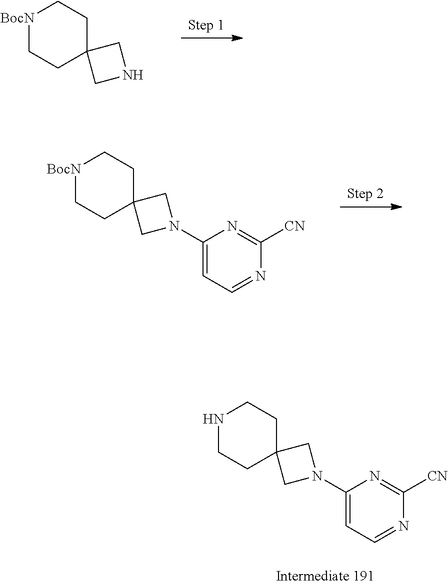 [SMILES: CC(C)(C)OC(=O)N1CCC2(CC1)CNC2 ; CC(C)(C)OC(=O)N1CCC2(CC1)CN(c1ccnc(C#N)n1)C2 ; N#Cc1nccc(N2CC3(CCNCC3)C2)n1]

### Step 1: Tert-butyl 2-(2-cyanopyrimidin-4-yl)-2,7-diazaspiro[3.5]nonane-7-carboxylate

To a solution of tert-butyl 2,7-diazaspiro[3.5]nonane-7-carboxylate (180 mg, 908 μmol) and 4-chloropyrimidine-2-carbonitrile (127 mg, 908 μmol) in ACN (10 mL) were added NaI (132 mg, 884 μmol) and K₂CO₃ (1.83 g, 13.3 mmol). The mixture was stirred at 80° C. for 2 hr. The reaction mixture was diluted with H₂O (50 mL) and extracted with EtOAc (50 mL×3). The combined organic layers were washed with brine (50 mL), dried over Na₂SO₄, filtered, and concentrated under reduced pressure. After purified by silica gel flash chromatography (Eluent of 0˜48% EtOAc in petroleum ether), tert-butyl 2-(2-cyanopyrimidin-4-yl)-2,7-diazaspiro[3.5]nonane-7-carboxylate (Intermediate, 1.3 g, yield: 89%) was obtained as a light yellow solid. MS: m/z=330.1 [M+H]⁺. ¹H NMR (400 MHz, Chloroform-d) δ 15 (d, J=6.0 Hz, 1H), 6.26 (d, J=6.0 Hz, 1H), 3.84 (s, 4H), 3.40 (s, 4H), 1.80-1.75 (m, 4H), 1.46 (s, 9H).

### Step 2: 4-(2,7-Diazaspiro[3.5]nonan-2-yl)pyrimidine-2-carbonitrile

To a solution of tert-butyl 2-(2-cyanopyrimidin-4-yl)-2,7-diazaspiro[3.5]nonane-7-carboxylate (120 mg, 364 μmol) in CH₂Cl₂ (5 mL) was added TFA (13.5 mmol, 1 mL). The mixture was stirred at 25° C. for 1 hr. The mixture was concentrated under reduced pressure to give 4-(2,7-diazaspiro[3.5]nonan-2-yl)pyrimidine-2-carbonitrile (Intermediate 191, 120 mg, TFA salt, crude) as a yellow oil. MS: m/z=230.0 [M+H]⁺.

### Intermediate 192: 4-(2,7-Diazaspiro[3.5]nonan-7-yl)pyrimidine-2-carbonitrile

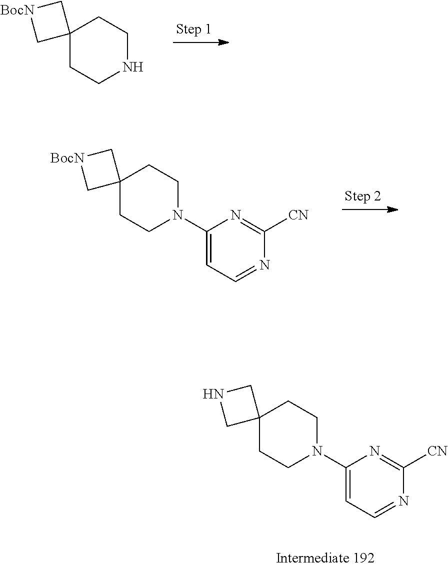 [SMILES: CC(C)(C)OC(=O)N1CC2(CCNCC2)C1 ; CC(C)(C)OC(=O)N1CC2(CCN(c3ccnc(C#N)n3)CC2)C1 ; N#Cc1nccc(N2CCC3(CC2)CNC3)n1]

### Step 1: Tert-butyl 7-(2-cyanopyrimidin-4-yl)-2,7-diazaspiro[3.5]nonane-2-carboxylate

To a solution of tert-butyl 2,7-diazaspiro[3.5]nonane-2-carboxylate (500 mg, 2.21 mmol) and 4-chloropyrimidine-2-carbonitrile (308 mg, 2.21 mmol) in ACN (10 mL) were added NaI (33 mg, 221 μmol) and K₂CO₃ (916 mg, 6.63 mmol). The mixture was stirred at 80° C. for 16 hr. The reaction mixture was diluted with H₂O (50 mL) and extracted with EtOAc (50 mL×3). The combined organic layers were washed with brine (50 mL), dried over Na₂SO₄, filtered and concentrated under reduced pressure, tert-butyl 7-(2-cyanopyrimidin-4-yl)-2,7-diazaspiro[3.5]nonane-2-carboxylate (720 mg, yield: 97%) was obtained as a light yellow solid. MS: m/z=330.2 [M+H]+. ¹H NMR (400 MHz, Dimethylsulfoxide-d₆) δ 0.22 (d, J=6.0 Hz, 1H), 7.13 (d, J=6.8 Hz, 1H), 3.65-3.53 (m, 8H), 1.74-1.69 (m, 4H), 1.38 (s, 9H).

### Step 2: 4-(2,7-Diazaspiro[3.5]nonan-7-yl)pyrimidine-2-carbonitrile

To a solution of tert-butyl 7-(2-cyanopyrimidin-4-yl)-2,7-diazaspiro[3.5]nonane-2-carboxylate (110 mg, 334 μmol) in CH₂Cl₂ (5 mL) was added TFA (13.5 mmol, 1 mL). The mixture was stirred at 25° C. for 1 hr. The mixture was concentrated under reduced pressure to give 4-(2,7-diazaspiro[3.5]nonan-7-yl)pyrimidine-2-carbonitrile (Intermediate 192, 110 mg, TFA salt, crude) as a yellow oil. MS: m/z=230.0 [M+H]⁺.

### Intermediate 193: 3-(5-Bromo-3-(4-(chloromethyl)phenyl)-3H-imidazo[4,5-b]pyridin-2-yl)pyridin-2-amine

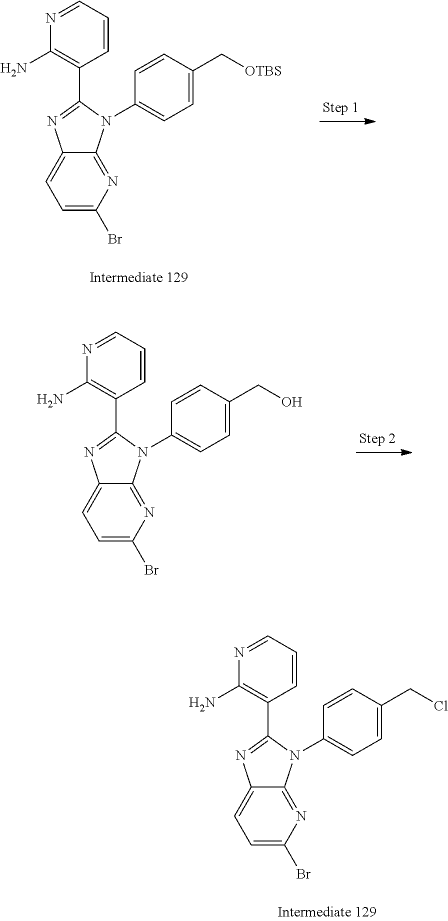 [SMILES: CC(C)(C)[Si](C)(C)OCc1ccc(-n2c(-c3cccnc3N)nc3ccc(Br)nc32)cc1 ; Nc1ncccc1-c1nc2ccc(Br)nc2n1-c1ccc(CO)cc1 ; Nc1ncccc1-c1nc2ccc(Br)nc2n1-c1ccc(CCl)cc1]

### Step 1: (4-(2-(2-Aminopyridin-3-yl)-5-bromo-3H-imidazo[4,5-b]pyridin-3-yl)phenyl)methanol

To a solution of Intermediate 129 (5 g, 9.79 mmol) in THF (50 mL) was added TBAF (1 M, 14.7 mL). The mixture was degassed and purged with N₂ three times and stirred at 25° C. for 2 hr under N₂. The reaction mixture was quenched with saturated NH₄Cl (100 mL), then extracted with EtOAc (100 mL×2). The combined organic layers were washed with brine (200 mL×3), dried over Na₂SO₄, filtered and concentrated under reduced pressure, (4-(2-(2-aminopyridin-3-yl)-5-bromo-3H-imidazo[4,5-b]pyridin-3-yl)phenyl)methanol (3.88 g, crude) was obtained as a yellow solid which was used directly into the next step. MS: m/z=395.9, 397.9 [M+H]⁺.

### Step 2: 3-(5-Bromo-3-(4-(chloromethyl)phenyl)-3H-imidazo[4,5-b]pyridin-2-yl)pyridin-2-amine

To a solution of (4-(2-(2-aminopyridin-3-yl)-5-bromo-3H-imidazo[4,5-b]pyridin-3-yl)phenyl)methanol (3.88 g, 9.79 mmol) in CH₂Cl₂ (50 mL) was added SOCl₂ (7.11 mL, 97.9 mmol). The mixture was degassed and purged with N₂ three times and stirred at 25° C. for 2 hr under N₂. The reaction mixture was concentrated under reduced pressure. 3-(5-Bromo-3-(4-(chloromethyl)phenyl)-3H-imidazo[4,5-b]pyridin-2-yl)pyridin-2-amine (Intermediate 193, 4.06 g, crude) was obtained as a gray solid which was used directly into the next step. MS: m/z=414.0, 416.0 [M+H]⁺.

### Intermediate 194: 3-(5-Bromo-3-(4-(chloromethyl-d₂)phenyl)-3H-imidazo[4,5-b]pyridin-2-yl)pyridin-2-amine

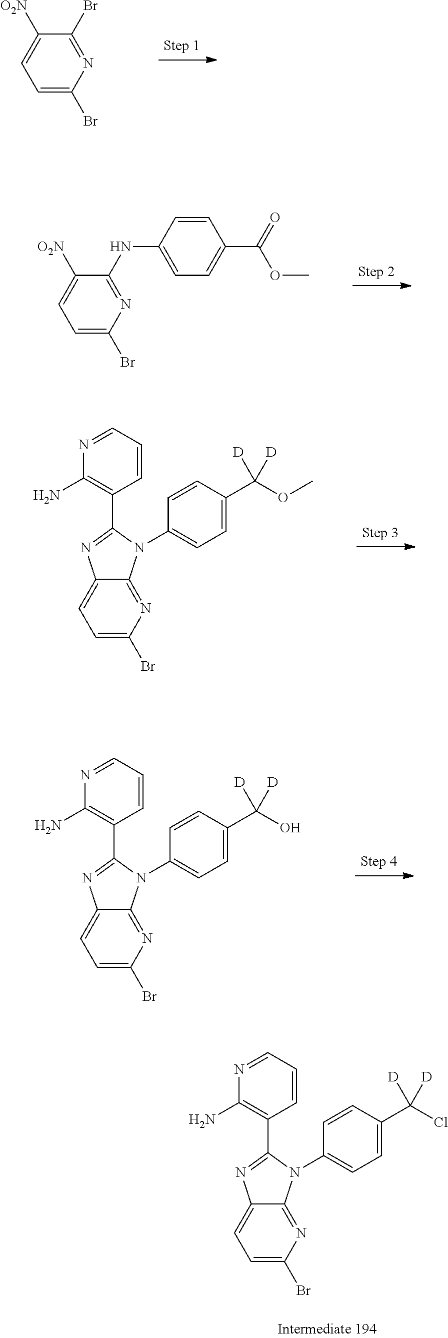 [SMILES: O=[N+]([O-])c1ccc(Br)nc1Br ; COC(=O)c1ccc(Nc2nc(Br)ccc2[N+](=O)[O-])cc1 ; [2H]C([2H])(OC)c1ccc(-n2c(-c3cccnc3N)nc3ccc(Br)nc32)cc1 ; [2H]C([2H])(O)c1ccc(-n2c(-c3cccnc3N)nc3ccc(Br)nc32)cc1 ; [2H]C([2H])(Cl)c1ccc(-n2c(-c3cccnc3N)nc3ccc(Br)nc32)cc1]

### Step 1: Methyl 4-((6-bromo-3-nitropyridin-2-yl)amino)benzoate

To a solution of 2,6-dibromo-3-nitropyridine (10 g, 35.5 mmol) and methyl 4-aminobenzoate (5.36 g, 35.5 mmol) in 1,4-dioxane (100 mL) was added DIEA (18.5 mL, 106 mmol). The mixture was degassed and purged with N₂ three times and stirred at 40° C. for 16 hr under N₂. The reaction mixture was concentrated under reduced pressure. The crude product was triturated with EtOAc at 25° C. for 30 mins. Methyl 4-((6-bromo-3-nitropyridin-2-yl)amino)benzoate (9.2 g, yield: 67%) was obtained as a red solid. MS: m/z=351.7, 353.7 [M+H]⁺. ¹H NMR (400 MHz, Chloroform-d) δ 10.38 (s, 1H), 8.35 (d, J=8.8 Hz, 1H), 8.07 (d, J=8.8 Hz, 2H), 7.77 (d, J=8.8 Hz, 2H), 7.05 (d, J=8.8 Hz, 1H), 3.91 (s, 3H).

### Step 2: 4-(2-(2-Aminopyridin-3-yl)-5-bromo-3H-imidazo[4,5-b]pyridin-3-yl)benzoate

To a solution of methyl 4-((6-bromo-3-nitropyridin-2-yl)amino)benzoate (4.5 g, 12.8 mmol) and 2-aminonicotinaldehyde (1.72 g, 14.1 mmol) in DMSO (50 mL) was added Na₂S₂O₄ (4.45 g, 25.6 mmol). The mixture was degassed and purged with N₂ three times and stirred at 100° C. for 16 hr under N₂. The reaction mixture was quenched with H₂O (200 mL) and then extracted with CH₂Cl₂ (100 mL×3). The combined organic layers were washed with brine (200 mL×2), dried over Na₂SO₄, filtered and concentrated under reduced pressure. After purified by silica gel flash chromatography (EtOAc in petroleum ether=1 to 50%). 4-(2-(2-Aminopyridin-3-yl)-5-bromo-3H-imidazo[4,5-b]pyridin-3-yl)benzoate (2.2 g, yield: 36.5%) was obtained as a yellow solid. MS: m/z=423.8, 425.8 [M+H]⁺. ¹H NMR (400 MHz, Chloroform-0) 8.20 (d, J=8.4 Hz, 2H), 8.06 (dd, J=4.4, 1.2 Hz, 1H), 7.95 (d, J=8.4 Hz, 1H), 7.50-7.43 (m, 3H), 7.02 (dd, J=8.0, 1.6 Hz, 1H), 6.69 (br s, 2H), 6.37 (dd, J=7.6, 4.8 Hz, 1H), 3.97 (s, 3H).

### Step 3: (4-(2-(2-Aminopyridin-3-yl)-5-bromo-3H-imidazo[4,5-b]pyridin-3-yl)phenyl)methan-d₂-ol

To a solution of methyl 4-(2-(2-aminopyridin-3-yl)-5-bromo-3H-imidazo[4,5-b]pyridin-3-yl)benzoate (300 mg, 707 μmol) in THF (30 mL) was added LiAlD₄ (65.1 mg, 1.41 mmol). The mixture was degassed and purged with N₂ three times and stirred at 0° C. for 1 hr under N₂. The reaction mixture was quenched with Na₂SO₄·10H₂O (1 g) at 0° C., and then filtered and concentrated under reduced pressure. (4-(2-(2-Aminopyridin-3-yl)-5-bromo-3H-imidazo[4,5-b]pyridin-3-yl)phenyl)methan-d₂-ol (190 mg, crude) was obtained as a yellow solid which was used directly into the next step. MS: m/z=397.9, 399.9 [M+H]⁺.

### Step 4: 3-(5-Bromo-3-(4-(chloromethyl-d₂)phenyl)-3H-imidazo[4,5-b]pyridin-2-yl)pyridin-2-amine

To a solution of (4-(2-(2-aminopyridin-3-yl)-5-bromo-3H-imidazo[4,5-b]pyridin-3-yl)phenyl)methan-d₂-ol (190 mg, 477 μmol) in CH₂Cl₂ (2 mL) was added SOCl₂ (347 μL, 4.77 mmol). The mixture was degassed and purged with N₂ three times and stirred at 25° C. for 2 hr under N₂. The reaction mixture was concentrated under reduced pressure. 3-(5-Bromo-3-(4-(chloromethyl-d₂)phenyl)-3H-imidazo[4,5-b]pyridin-2-yl)pyridin-2-amine (Intermediate 194, 199 mg, crude) was obtained as a gray solid, which was used directly into the next step. MS: m/z=416.0, 418.0 [M+H]⁺.

### Intermediate 195: 3-(3-(4-(Chloromethyl-d₂)phenyl)-5-(4-fluorophenyl)-3H-imidazo[4,5-b]pyridin-2-yl)pyridin-2-amine

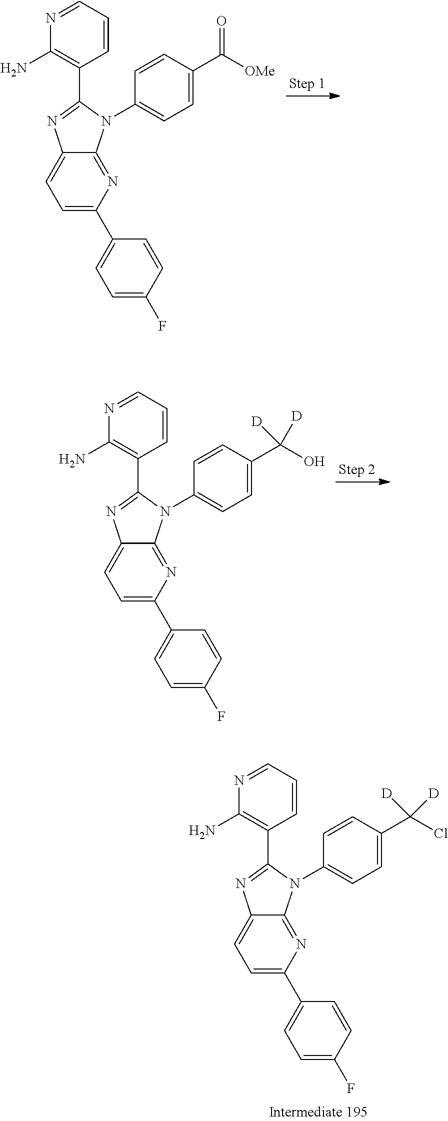 [SMILES: COC(=O)c1ccc(-n2c(-c3cccnc3N)nc3ccc(-c4ccc(F)cc4)nc32)cc1 ; [2H]C([2H])(O)c1ccc(-n2c(-c3cccnc3N)nc3ccc(-c4ccc(F)cc4)nc32)cc1 ; [2H]C([2H])(Cl)c1ccc(-n2c(-c3cccnc3N)nc3ccc(-c4ccc(F)cc4)nc32)cc1]

### Step 1: (4-(2-(2-Aminopyridin-3-yl)-5-(4-fluorophenyl)-3H-imidazo[4,5-b]pyridin-3-yl)phenyl)methan-d₂-ol

To a solution of methyl 4-(2-(2-aminopyridin-3-yl)-5-(4-fluorophenyl)-3H-imidazo[4,5-b]pyridin-3-yl)benzoate (refer to Intermediate 107 for detail procedures, 900 mg, 2.0 mmol) in THF (3 mL) was added LiAlD₄ (155 mg, 4.1 mmol) at 0° C. The reaction mixture was stirred at 25° C. for 1 hr. The reaction mixture was quenched with Na₂SO₄·10H₂O (203 mg) at 0° C. and filtered, concentrated under reduced pressure. The crude product (4-(2-(2-aminopyridin-3-yl)-5-(4-fluorophenyl)-3H-imidazo[4,5-b]pyridin-3-yl)phenyl)methan-d₂-ol (830 mg) was used into the next step without further purification. MS: m/z=414.1 [M+H]+. ¹⁹F NMR (400 MHz, Dimethylsulfoxide-d₆) δ −113.60

### Step 2: 3-(3-(4-(Chloromethyl-d₂)phenyl)-5-(4-fluorophenyl)-3H-imidazo[4,5-b]pyridin-2-yl)pyridin-2-amine

To a solution of (4-(2-(2-aminopyridin-3-yl)-5-(4-fluorophenyl)-3H-imidazo[4,5-b]pyridin-3-yl)phenyl)methan-d₂-ol (500 mg, 1.2 mmol) in CH₂Cl₂ (10 mL) was added SOCl₂ (719 mg, 6.0 mmol) at 25° C. The reaction mixture was stirred at 40° C. for 0.5 hr. The reaction mixture was concentrated under reduced pressure. The crude product 3-(3-(4-(chloromethyl-d₂)phenyl)-5-(4-fluorophenyl)-3H-imidazo[4,5-b]pyridin-2-yl)pyridin-2-amine (Intermediate 195, 500 mg, yield: 74%) was used into the next step without further purification. MS: m/z=432.0 [M+H]+. ¹⁹F NMR (400 MHz, Dimethylsulfoxide-d₆) δ −113.05.

### Intermediate 196: 3-(3-(4-(Chloromethyl)phenyl)-5,7-dimethyl-3H-imidazo[4,5-b]pyridin-2-yl)pyridin-2-amine

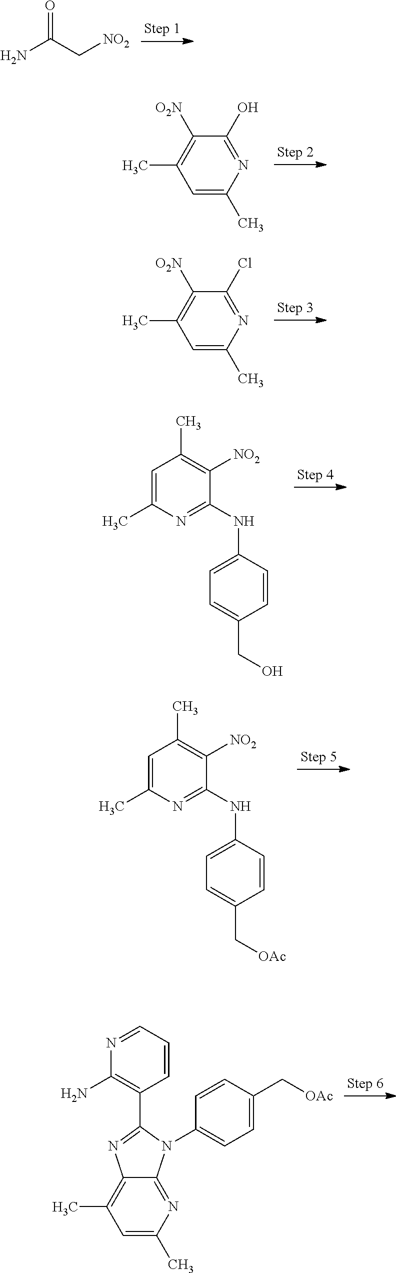

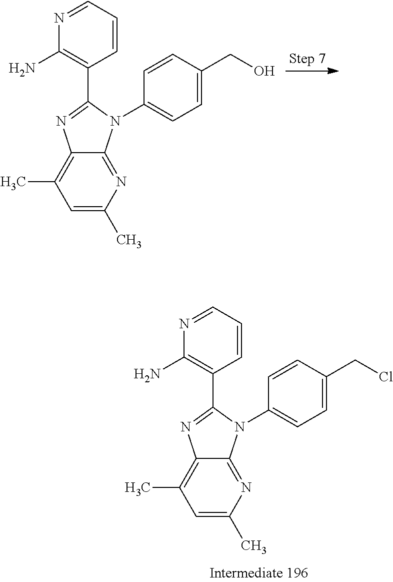 [SMILES: Cc1cc(C)c2nc(-c3cccnc3N)n(-c3ccc(CO)cc3)c2n1 ; Cc1cc(C)c2nc(-c3cccnc3N)n(-c3ccc(CCl)cc3)c2n1]

### Step 1: 4,6-Dimethyl-3-nitropyridin-2-ol

To a solution of 2-nitroacetamide (12.3 g, 118 mmol) in H₂O (120 mL) were added pentane-2,4-dione (14.4 g, 143 mmol), Py (3.09 g, 39.1 mmol), AcOH (2.35 g, 39.2 mmol). The mixture was stirred at 25° C. for 7 days. The mixture was filtered and the filter cake was concentrated under reduced pressure to give 4,6-dimethyl-3-nitropyridin-2-ol (4.5 g, yield: 20%) as a light-yellow solid. ¹H NMR (400 MHz, Dimethylsulfoxide-d₆) δ 12.14 (s, 1H), 6.08 (s, 1H), 2.20 (s, 3H), 2.14 (s, 3H).

### Step 2: 2-Chloro-4,6-dimethyl-3-nitropyridine

To a solution of 4,6-dimethyl-3-nitropyridin-2-ol (4 g, 23.8 mmol) in POCl₃ (65.8 g, 429 mmol) was stirred at 100° C. for 2 hr under N₂ atmosphere. The reaction mixture was quenched with H₂O (300 mL) at 25° C., and then extracted with EtOAc (500 mL×2). The combined organic layers were washed with brine (300 mL×2), dried over Na₂SO₄, filtered, and concentrated under reduced pressure. After purified by silica gel flash chromatography (Eluent of 0%˜6% EtOAc in petroleum ether), 2-chloro-4,6-dimethyl-3-nitropyridine (3.8 g, yield: 85%) was obtained as a light-yellow solid. MS: m/z=186.9 [M+H]⁺. ¹H NMR (400 MHz, Dimethylsulfoxide-d₆) δ 7.49 (s, 1H), 2.50 (s, 3H), 2.32 (s, 3H).

### Step 3: (4-((4,6-Dimethyl-3-nitropyridin-2-yl)amino)phenyl)methanol

To a solution of 2-chloro-4,6-dimethyl-3-nitropyridine (2.4 g, 12.8 mmol) and (4-aminophenyl)methanol (1.74 g, 14.1 mmol) in 1,4-dioxane (30 mL) and was added DIEA (4.99 g, 38.6 mmol). The mixture was stirred at 110° C. for 48 hr. The reaction mixture was diluted H₂O (300 mL) and extrated with EtOAc (500 mL×2). The combined organic layers were washed with brine (300 mL×2), dried over Na₂SO₄, filtered and concentrated under reduced pressure to give (4-((4,6-dimethyl-3-nitropyridin-2-yl)amino)phenyl)methanol (3.3 g, yield: 78%) was obtained as a red solid. MS: m/z=274.0 [M+H]⁺.

### Step 4: 4-((4,6-Dimethyl-3-nitropyridin-2-yl)amino)benzyl acetate

To a mixture of (4-((4,6-dimethyl-3-nitropyridin-2-yl)amino)phenyl)methanol (3 g, 10.9 mmol), DMAP (134 mg, 1.10 mmol) and TEA (3.33 g, 32.9 mmol) in CH₂Cl₂ (50 mL) was added acetic anhydride (1.12 g, 10.9 mmol), then degassed and purged with N₂ three times and stirred at 0° C. for 2 hr under N₂ atmosphere. The reaction mixture was diluted with H₂O (200 mL) and extracted with EtOAc (200 mL×3). The combined organic layers were washed with brine (200 mL), dried over Na₂SO₄, filtered and concentrated under reduced pressure. After purified by silica gel flash chromatography (Eluent of 0˜50% EtOAc in petroleum ether), 4-((4,6-dimethyl-3-nitropyridin-2-yl)amino)benzyl acetate (970 mg, yield: 28%) was obtained as a yellow solid. MS: m/z=316.0 [M+H]⁺. ¹H NMR (400 MHz, Chloroform-d) δ 9.59 (s, 1H), 7.64 (d, J=8.4 Hz, 2H), 7.35 (d, J=8.4 Hz, 2H), 6.56 (s, 1H), 5.09 (s, 2H), 2.55 (s, 3H), 2.44 (s, 3H), 2.10 (s, 3H).

### Step 5: 4-(2-(2-Aminopyridin-3-yl)-5,7-dimethyl-3H-imidazo[4,5-b]pyridin-3-yl)benzyl acetate

A mixture of 4-((4,6-dimethyl-3-nitropyridin-2-yl)amino)benzyl acetate (970 mg, 3.08 mmol), 2-aminonicotinaldehyde (413 mg, 3.38 mmol) and Na₂S₂O₄ (2.14 g, 12.3 mmol) in DMSO (100 mL) was degassed and purged with N₂ three times, and then was stirred at 100° C. for 16 hr under N₂ atmosphere. The reaction mixture was diluted with H₂O (100 mL) and extracted with EtOAc (100 mL×3). The combined organic layers were washed with brine (200 mL), dried over Na₂SO₄, filtered and concentrated under reduced pressure. After purified by silica gel flash chromatography (Eluent of 0˜50% EtOAc in petroleum ether), 4-(2-(2-aminopyridin-3-yl)-5,7-dimethyl-3H-imidazo[4,5-b]pyridin-3-yl)benzyl acetate (550 mg, yield: 44%) was obtained as a yellow solid. MS: m/z=388.1 [M+H]+. ¹H NMR (400 MHz, Dimethylsulfoxide-d₆) δ 7.97 (dd, J=4.8, 2.0 Hz, 1H), 7.51 (d, J=8.4 Hz, 2H), 7.40 (d, J=8.4 Hz, 2H), 7.17 (dd, J=7.6, 1.6 Hz, 1H), 7.10 (s, 1H), 6.93 (s, 2H), 6.40 (dd, J=7.6, 4.8 Hz, 1H), 5.16 (s, 2H), 2.62 (s, 3H), 2.47 (s, 3H), 2.11 (s, 3H).

### Step 6: (4-(2-(2-Aminopyridin-3-yl)-5,7-dimethyl-3H-imidazo[4,5-b]pyridin-3-yl)phenyl)methanol

To a mixture of 4-(2-(2-aminopyridin-3-yl)-5,7-dimethyl-3H-imidazo[4,5-b]pyridin-3-yl)benzyl acetate (500 mg, 1.29 mmol) in MeOH (15 mL), THF (15 mL) and H₂O (7 mL) was added K₂CO₃ (178 mg, 1.29 mmol). The mixture was stirred at 25° C. for 0.5 hr. The mixture was filtered, concentrated under reduced pressure, (4-(2-(2-aminopyridin-3-yl)-5,7-dimethyl-3H-imidazo[4,5-b]pyridin-3-yl)phenyl)methanol (400 mg, yield: 80%) was obtained as a yellow solid. MS: m/z=346.1 [M+H]⁺.

### Step 7: 3-(3-(4-(Chloromethyl)phenyl)-5,7-dimethyl-3H-imidazo[4,5-b]pyridin-2-yl)pyridin-2-amine

To a solution of (4-(2-(2-aminopyridin-3-yl)-5,7-dimethyl-3H-imidazo[4,5-b]pyridin-3-yl)phenyl)methanol (400 mg, 1.16 mmol) in CH₂Cl₂ (15 mL) was added SOCl₂ (827 mg, 6.95 mmol). The mixture was stirred at 40° C. for 1 hr. The mixture was concentrated to give 3-(3-(4-(chloromethyl)phenyl)-5,7-dimethyl-3H-imidazo[4,5-b]pyridin-2-yl)pyridin-2-amine (Intermediate 196, 350 mg, yield: 88%) as a yellow solid, which was used in the next step without further purification. MS: m/z=364.0 [M+H]⁺.

### Intermediate 197: 3-(3-(4-(chloromethyl)phenyl)-5-(pyridin-2-yl)-3H-imidazo[4,5-b]pyridin-2-yl)pyridin-2-amine

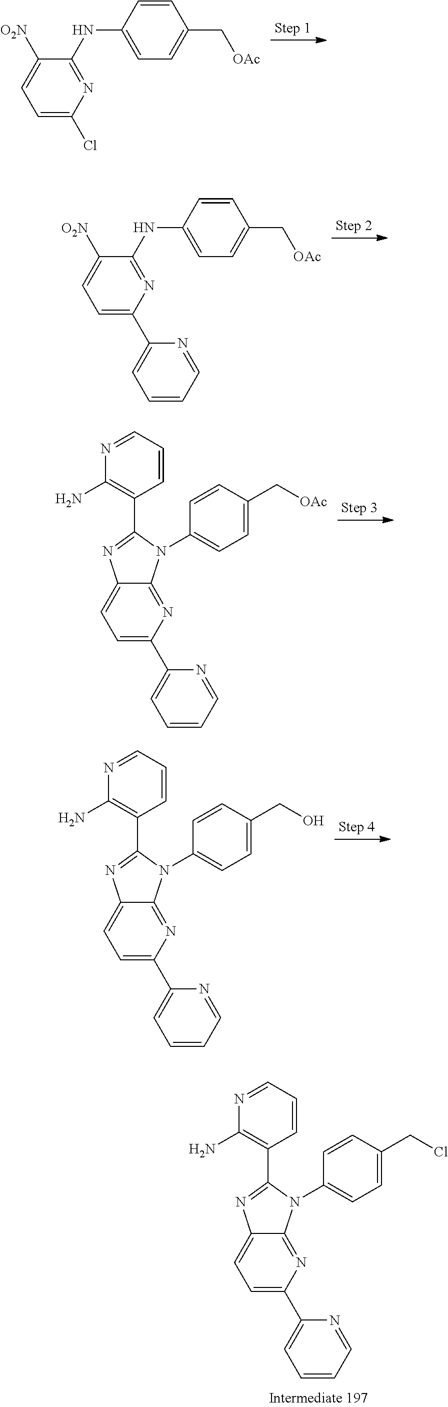

### Step 1: 4-((5-Nitro-[2,2′-bipyridin]-6-yl)amino)benzyl acetate

To a solution of 4-((6-chloro-3-nitropyridin-2-yl)amino)benzyl acetate (refer to Intermediate 178 for detail procedures, 500 mg, 1.6 mmol) in 1,4-dioxane (10 mL) were added CuBr (167 mg, 1.2 mmol), Cs₂CO₃ (1.5 g, 4.7 μmol), pyridin-2-ylboronic acid (573 mg, 4.7 mmol) and cataCXium A PdG3 (113 mg, 155 μmol) at 25° C., the reaction mixture was degassed and purged with N₂ three times. The mixture was stirred at 100° C. for 4 hr under N₂. The reaction mixture was poured into H₂O (50 mL), then extracted with CH₂Cl₂ (20 mL×2). The combined organic layers were washed with brine (40 mL), dried over anhydrous Na₂SO₄, filtered and concentrated. After purified by silica gel flash chromatography (Eluent of 0˜45% EtOAc in petroleum ether), 4-((5-nitro-[2,2′-bipyridin]-6-yl)amino)benzyl acetate (300 mg, yield: 43%) was obtained as a yellow solid. MS: m/z=365.0 [M+H]⁺. ¹H NMR (400 MHz, Dimethylsulfoxide-d₆) δ 10.11 (s, 1H), 8.76 (d, J=4.0 Hz, 1H), 8.70 (d, J=8.8 Hz, 1H), 8.20 (d, J=8.0 Hz, 1H), 8.05-7.95 (m, 2H), 7.81-7.75 (m, 2H), 7.56 (dd, J=7.2, 4.8 Hz, 1H), 7.45 (d, J=8.4 Hz, 2H), 5.10 (s, 2H), 2.09 (s, 3H).

### Step 2: 4-(2-(2-Aminopyridin-3-yl)-5-(pyridin-2-yl)-3H-imidazo[4,5-b]pyridin-3-yl)benzyl acetate

To a solution of 4-((5-nitro-[2,2′-bipyridin]-6-yl)amino)benzyl acetate (300 mg, 823 μmol) in DMSO (10 mL) were added 2-aminonicotinaldehyde (121 mg, 988 μmol) and Na₂S₂O₄ (573 mg, 3.3 mmol) at 25° C. The mixture was stirred at 100° C. for 12 hr. The reaction mixture was poured into H₂O (100 mL). The mixture was extracted with CH₂Cl₂ (50 mL×2). The combined organic layers were washed with 400 mL brine, dried over anhydrous Na₂SO₄, filtered and concentrated. After purified by silica gel flash chromatography (Eluent of 0˜3% MeOH in CH₂Cl₂), 4-(2-(2-aminopyridin-3-yl)-5-(pyridin-2-yl)-3H-imidazo[4,5-b]pyridin-3-yl)benzyl acetate (200 mg, yield: 56%) was obtained as a yellow solid. MS: m/z=437.0 [M+H]+. ¹H NMR (400 MHz, Dimethylsulfoxide-d₆) δ 8.67 (d, J=4.0 Hz, 1H), 8.47 (d, J=8.4 Hz, 1H), 8.32 (d, J=8.4 Hz, 1H), 8.19 (d, J=8.0 Hz, 1H), 8.02 (dd, J=4.8, 2.0 Hz, 1H), 7.94-7.87 (m, 1H), 7.59-7.51 (m, 4H), 7.40 (dd, J=7.2, 5.6 Hz, 1H), 7.25 (dd, J=7.6, 2.0 Hz, 1H), 6.90 (s, 2H), 6.44 (dd, J=7.6, 4.8 Hz, 1H), 5.19 (s, 2H), 2.12 (s, 3H).

### Step 3: (4-(2-(2-Aminopyridin-3-yl)-5-(pyridin-2-yl)-3H-imidazo[4,5-b]pyridin-3-yl)phenyl)methanol

To a solution of 4-(2-(2-aminopyridin-3-yl)-5-(pyridin-2-yl)-3H-imidazo[4,5-b]pyridin-3-yl)benzyl acetate (200 mg, 458 μmol) in H₂O (1 mL) and MeOH (2 mL) THF (2 mL) was added K₂C03 (63 mg, 458 μmol) at 25° C. The mixture was stirred at 25° C. for 1 hr. The reaction mixture was poured into H₂O (20 mL). The mixture was extracted with CH₂Cl₂ (20 mL×2). The combined organic layers were washed with brine (40 mL), dried over anhydrous Na₂SO₄, filtered and concentrated, (4-(2-(2-aminopyridin-3-yl)-5-(5-fluoropyridin-2-yl)-³H-imidazo[4,5-b]pyridin-3-yl)phenyl)methanol (300 mg, crude) was obtained as a yellow solid. MS: m/z=395.3 [M+H]⁺. ¹H NMR (400 MHz, Dimethylsulfoxide-d₆) δ 8.64-8.58 (m, 1H), 8.44 (d, J=8.4 Hz, 1H), 8.30-8.20 (m, 2H), 7.99 (dd, J=4.8, 1.6 Hz, 1H), 7.87-7.80 (m, 1H), 7.57 (d, J=8.4 Hz, 2H), 7.47 (d, J=8.4 Hz, 2H), 7.40-7.32 (m, 2H), 6.48 (dd, J=7.6, 5.2 Hz, 1H), 4.72 (s, 2H).

### Step 4: 3-(3-(4-(Chloromethyl)phenyl)-5-(pyridin-2-yl)-3H-imidazo[4,5-b]pyridin-2-yl)pyridin-2-amine

To a solution of (4-(2-(2-aminopyridin-3-yl)-5-(5-fluoropyridin-2-yl)-3H-imidazo[4,5-b]pyridin-3-yl)phenyl)methanol (180 mg, 456 μmol) in CH₂Cl₂ (5 mL) was added SOCl₂ (271 mg, 2.3 mmol) at 25° C. The mixture was stirred at 40° C. for 0.5 hr. The reaction mixture was concentrated under reduced pressure to give 3-(3-(4-(chloromethyl)phenyl)-5-(pyridin-2-yl)-3H-imidazo[4,5-b]pyridin-2-yl)pyridin-2-amine (Intermediate 197, 188 mg, crude) as a yellow solid, which was used in the next step without further purification. MS: m/z=413.0 [M+H]⁺.

### Intermediate 198: 3-(3-(4-(Chloromethyl)phenyl)-5-(pyrrolidin-1-yl)-3H-imidazo[4,5-b]pyridin-2-yl)pyridin-2-amine

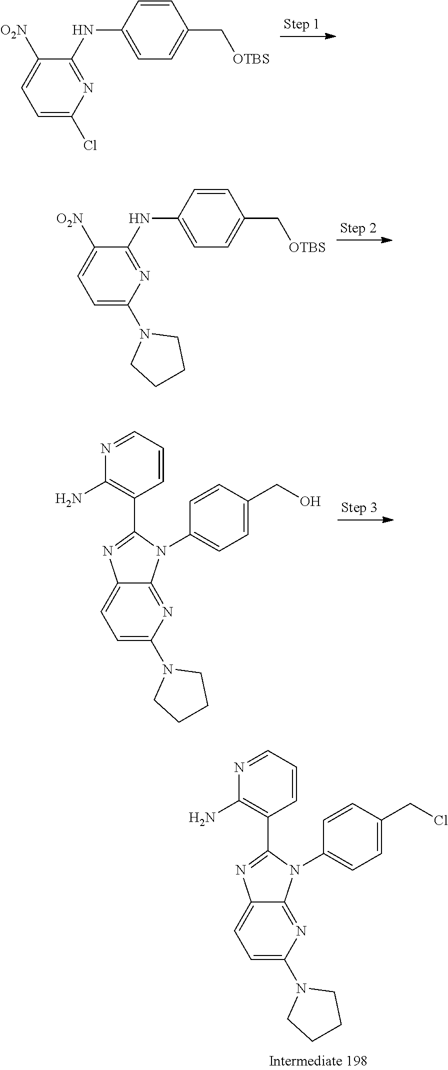 [SMILES: CC(C)(C)[Si](C)(C)OCc1ccc(Nc2nc(Cl)ccc2[N+](=O)[O-])cc1 ; CC(C)(C)[Si](C)(C)OCc1ccc(Nc2nc(N3CCCC3)ccc2[N+](=O)[O-])cc1 ; Nc1ncccc1-c1nc2ccc(N3CCCC3)nc2n1-c1ccc(CO)cc1 ; Nc1ncccc1-c1nc2ccc(N3CCCC3)nc2n1-c1ccc(CCl)cc1]

### Step 1: N-(4-(((Tert-butyldimethylsilyl)oxy)methyl)phenyl)-3-nitro-6-(pyrrolidin-1-yl)pyridin-2-amine

To a solution of N-(4-(((tert-butyldimethylsilyl)oxy)methyl)phenyl)-6-chloro-3-nitropyridin-2-amine (refer to Intermediate 105 for detail procedures, 3 g, 7.62 mmol) in MeCN (50 mL) were added DIEA (2.95 g, 22.9 mmol) and pyrrolidine (1.23 g, 11.4 mmol, HCl). The mixture was stirred at 90° C. for 12 hr. The reaction mixture was concentrated. The residue was dissolved in EtOAc (50 mL), washed with brine (50 mL×2), dried over anhydrous Na₂SO₄, filtered and concentrated. N-(4-(((Tert-butyldimethylsilyl)oxy)methyl)phenyl)-3-nitro-6-(pyrrolidin-1-yl)pyridin-2-amine (3 g, yield: 92%) was obtained as a brown solid, which was used directly into the next step. MS: m/z=429.1 [M+H]⁺.

### Step 2: (4-(2-(2-Aminopyridin-3-yl)-5-(pyrrolidin-1-yl)-3H-imidazo[4,5-b]pyridin-3-yl)phenyl)methanol

To a solution of N-(4-(((tert-butyldimethylsilyl)oxy)methyl)phenyl)-3-nitro-6-(pyrrolidin-1-yl)pyridin-2-amine (1.5 g, 3.50 mmol) in DMSO (30 mL) were added Na₂S₂O₄ (1.83 g, 10.5 mmol) and 2-aminonicotinaldehyde (470 mg, 3.85 mmol). The mixture was stirred at 100° C. for 12 hr. Saturated NaHCO₃ (50 mL) was added to the mixture and the mixture was extracted with CH₂Cl₂ (50 mL×2). The combined organic layers were washed with brine (50 mL), dried over anhydrous Na₂SO₄, filtered and concentrated. After purified by prep-HPLC (column: Xtimate C18 150×40 mm×10 μm; mobile phase. [water (NH₃H₂O+NH₄HCO₃)-ACN]; gradient: 35%-65% B over 8 min), (4-(2-(2-aminopyridin-3-yl)-5-(pyrrolidin-1-yl)-3H-imidazo[4,5-b]pyridin-3-yl)phenyl)methanol (200 mg, yield: 15%) was obtained as a yellow solid. MS: m/z=387.2 [M+H]+. ¹H NMR (400 MHz, Dimethylsulfoxide-d₆) δ 7.96-7.86 (m, 2H), 7.43 (d, J=8.0 Hz, 2H), 7.35-7.28 (m, 2H), 7.11-6.98 (m, 3H), 6.49 (d, J=8.8 Hz, 1H), 6.33 (dd, J=7.6, 4.8 Hz, 1H), 5.38-5.30 (m, 1H), 4.57 (d, J=5.6 Hz, 2H), 2.59-2.52 (m, 2H), 2.49-2.47 (m, 2H), 1.96-1.87 (m, 4H).

### Step 3: 3-(3-(4-(Chloromethyl)phenyl)-5-(pyrrolidin-1-yl)-3H-imidazo[4,5-b]pyridin-2-yl)pyridin-2-amine

To a solution of (4-42-(2-aminopyridin-3-yl)-5-(pyrrolidin-1-yl)-3H-imidazo[4,5-b]pyridin-3-yl)phenyl)methanol (100 mg, 259 μmol) in CH2Cl2 (3 mL) was added SOCl₂ (92.4 mg, 776 μmol) at 20° C. The mixture was stirred at 20° C. for 2 hr. The reaction mixture was concentrated. 3-(3-(4-(Chloromethyl)phenyl)-5-(pyrrolidin-1-yl)-3H-imidazo[4,5-b]pyridin-2-yl)pyridin-2-amine (Intermediate 198, 105 mg, yield: 100%) was obtained as a yellow solid, which was used directly into the next step. MS: m/z=405.1 [M+H]⁺.

### Intermediate 199: 3-(3-(4-(Chloromethyl)phenyl)-5-(2-methoxypyridin-4-yl)-3H-imidazo[4,5-b]pyridin-2-yl)pyridin-2-amine

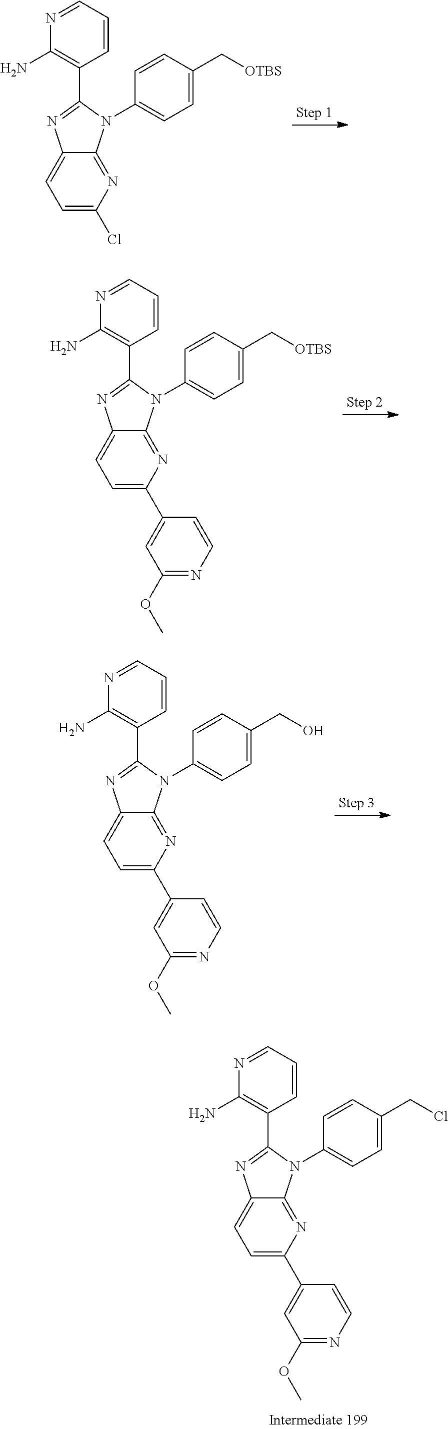

### Step 1: 3-(3-(4-(((tert-Butyldimethylsilyl)oxy)methyl)phenyl)-5-(2-methoxypyridin-4-yl)-3H-imidazo[4,5-b]pyridin-2-yl)pyridin-2-amine

To a solution of 3-(3-(4-(((tert-butyldimethylsilyl)oxy)methyl)phenyl)-5-chloro-3H-imidazo[4,5-b]pyridin-2-yl)pyridin-2-amine (refer to Intermediate 117 for detail procedures, 0.5 g, 1.07 mmol) in 1,4-dioxane (5 mL), H₂O (1 mL) were added (2-methoxypyridin-4-yl)boronic acid (230 mg, 1.50 mmol), Pd(dppf)Cl₂ (78.5 mg, 107 μmol), and CS₂CO₃ (445 mg, 1.37 mmol). The mixture was degassed and purged with N₂ three times, and stirred at 100° C. for 2 hr under N₂ atmosphere. The mixture was quenched with H₂O (20 mL) and extracted with EtOAc (8 mL×3). The combined organic layers were washed with brine (20 mL), dried over Na₂SO₄, filtered and concentrated under reduced pressure. After purified by silica gel flash chromatography (Eluent of 0˜62% EtOAc in petroleum ether), 3-(3-(4-(((tert-butyldimethylsilyl)oxy)methyl)phenyl)-5-(2-methoxypyridin-4-yl)-3H-imidazo[4,5-b]pyridin-2-yl)pyridin-2-amine (323 mg, yield: 88%) was obtained as a black brown solid. MS: m/z=539.2 [M+H]⁺.

### Step 2: (4-(2-(2-Aminopyridin-3-yl)-5-(2-methoxypyridin-4-yl)-3H-imidazo[4,5-b]pyridin-3-yl)phenyl)methanol

To a solution of 3-(3-(4-(((tert-butyldimethylsilyl)oxy)methyl)phenyl)-5-(2-methoxypyridin-4-yl)-3H-imidazo[4,5-b]pyridin-2-yl)pyridin-2-amine (323 mg, 600 μmol) in THE (5 mL) was added TBAF (1 M in THF, 959 μL). The mixture was stirred at 25° C. for 1 hr. The reaction was quenched with H₂O (10 mL) and extracted with EtOAc (8 mL×2). The combined organic layers were washed with brine, filtered and concentrated. (4-(2-(2-Aminopyridin-3-yl)-5-(2-methoxypyridin-4-yl)-3H-imidazo[4,5-b]pyridin-3-yl)phenyl)methanol (254 mg, yield: 97%) was obtained as a brown solid. MS: m/z=425.1 [M+H]⁺.

### Step 3: 3-(3-(4-(Chloromethyl)phenyl)-5-(2-methoxypyridin-4-yl)-3H-imidazo[4,5-b]pyridin-2-yl)pyridin-2-amine

To a solution of (4-(2-(2-aminopyridin-3-yl)-5-(2-methoxypyridin-4-yl)-3H-imidazo[4,5-b]pyridin-3-yl)phenyl)methanol (500 mg, 1.18 mmol) in CH₂Cl₂ (5 mL) was added SOCl₂ (257 μL, 3.53 mmol). The mixture was stirred at 25° C. for 1 hr. The reaction mixture was concentrated. 3-(3-(4-(Chloromethyl)phenyl)-5-(2-methoxypyridin-4-yl)-3H-imidazo[4,5-b]pyridin-2-yl)pyridin-2-amine (Intermediate 199, 500 mg, yield: 94%) was obtained as a black solid, which was used in the next step without further purification. MS: m/z=443.2 [M+H]⁺.

### Intermediate 200: 3-(3-(4-(Chloromethyl)phenyl)-5-(5-methoxypyridin-3-yl)-3H-imidazo[4,5-b]pyridin-2-yl)pyridin-2-amine

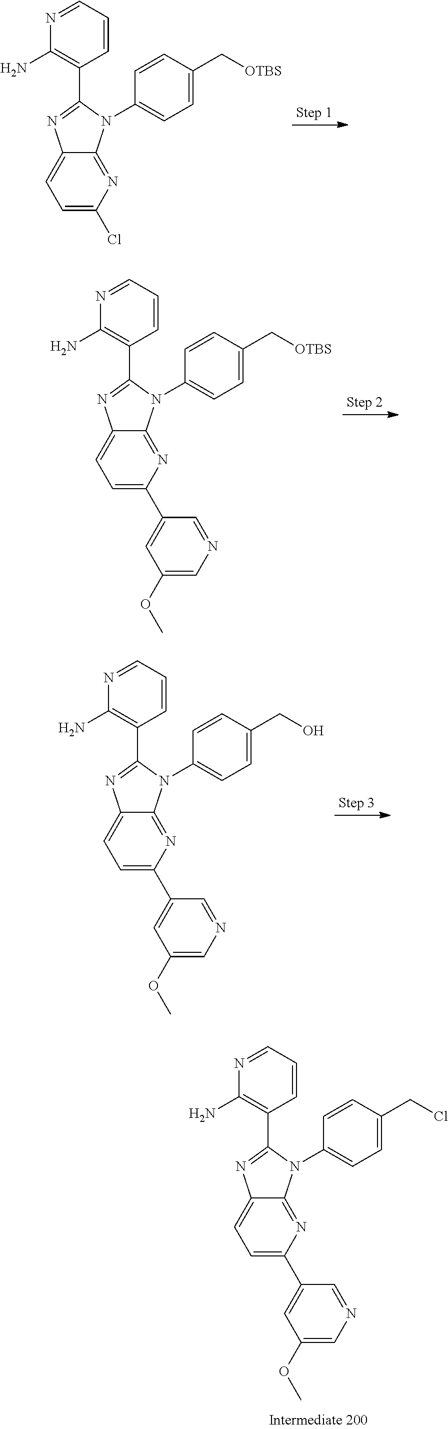

### Step 1: 3-(3-(4-(((Tert-butyldimethylsilyl)oxy)methyl)phenyl)-5-(5-methoxypyridin-3-yl)-3H-imidazo[4,5-b]pyridin-2-yl)pyridin-2-amine

To a solution of 3-(3-(4-(((tert-butyldimethylsilyl)oxy)methyl)phenyl)-5-chloro-3H-imidazo[4,5-b]pyridin-2-yl)pyridin-2-amine (refer to Intermediate 117 for detail procedures, 300 mg, 644 μmol) in 1,4-dioxane (3 mL), H₂O (0.6 mL) were added (5-methoxy-3-pyridyl)boronic acid (137 mg, 901 μmol), Pd(dppf)Cl₂ (47.1 mg, 64.3 μmol), Cs₂CO₃ (273 mg, 837 μmol). The mixture was stirred at 100° C. for 2 hr under N₂. After purified by silica gel flash chromatography (Eluent of 0%˜74% EtOAc in petroleum ether), 3-(3-(4-(((tert-butyldimethylsilyl)oxy)methyl)phenyl)-5-(5-methoxypyridin-3-yl)-3H-imidazo[4,5-b]pyridin-2-yl)pyridin-2-amine (275 mg, yield: 79.3%) was obtained as red solid. MS: m/z=539.3 [M+H]⁺. 1H NMR (400 MHz, Chloroform-d) δ 8.70 (d, J=1.6 Hz, 1H), 8.15 (d, J=2.8 Hz, 1H), 8.01 (d, J=8.4 Hz, 1H), 7.91 (dd, J=4.8, 1.6 Hz, 1H), 7.69-7.64 (m, 2H), 7.37-7.32 (m, 2H), 7.24 (s, 1H), 7.11 (s, 1H), 7.01 (dd, J=7.6, 1.6 Hz, 1H), 6.64 (br s, 2H), 6.24 (dd, J=7.6, 5.2 Hz, 1H), 4.71 (s, 2H), 3.76 (s, 3H), 0.84-0.81 (s, 9H). 0.00 (s, 6H).

### Step 2. (4-(2-(2-Aminopyridin-3-yl)-5-(5-methoxypyridin-3-yl)-3H-imidazo[4,5-b]pyridin-3-yl)phenyl)methanol

To a solution of 3-(3-(4-(((tert-butyldimethylsilyl)oxy)methyl)phenyl)-5-(5-methoxypyridin-3-yl)-3H-imidazo[4,5-b]pyridin-2-yl)pyridin-2-amine (275 mg, 510 μmol) in THF (1 mL) was added TBAF (1 M in THF, 817 pLL). The mixture was stirred at 25° C. for 1 hr under N₂. H₂O (30 mL) was add to the reaction mixture. The mixture was extracted with EtOAc (10 mL×3), washed with brine (10 mL), dried over Na₂SO₄, and concentrated. (4-(2-(2-Aminopyridin-3-yl)-5-(5-methoxypyridin-3-yl)-3H-imidazo[4,5-b]pyridin-3-yl)phenyl)methanol (217 mg, yield: 95%) was obtained as white solid. MS: m/z=425.3 [M+H]⁺.

### Step 3: 3-(3-(4-(Chloromethyl)phenyl)-5-(5-methoxypyridin-3-yl)-3H-imidazo[4,5-b]pyridin-2-yl)pyridin-2-amine

To a solution of (4-(2-(2-aminopyridin-3-yl)-5-(5-methoxypyridin-3-yl)-3H-imidazo[4,5-b]pyridin-3-yl)phenyl)methanol (217 mg, 510 μmol) in CH₂Cl₂ (5 mL) was added SOCl₂ (182 mg, 1.53 mmol). The mixture was stirred at 25° C. for 1 hr under N₂. The reaction mixture was concentrated to give 3-(3-(4-(chloromethyl)phenyl)-5-(5-methoxypyridin-3-yl)-3H-imidazo[4,5-b]pyridin-2-yl)pyridin-2-amine (Intermediate 200, 226 mg, yield. 97%) as a brown solid. MS: m/z=443.2 [M+H]⁺.

### Intermediate 201: N-Methyl-N-(3-(4,4,5,5-tetramethyl-1,3,2-dioxaborolan-2-yl)phenyl)acetamide

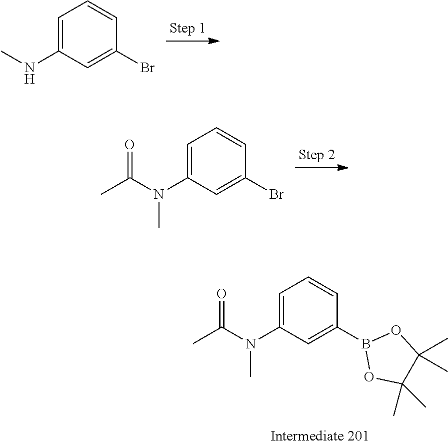

### Step 1: N-(3-Bromophenyl)-N-methylacetamide

To a solution of 3-bromo-N-methyl-aniline (500 mg, 2.69 mmol) in CH₂Cl₂ (10 mL) were added TEA (816 mg, 8.06 mmol) and AcCl (316 mg, 4.03 mmol) at 25° C. This mixture was stirred at 25° C. for 1 hr. The mixture was quenched with H₂O (30 mL) and extracted with EtOAc (20 mL×3). The combined organic layers were washed with brine (50 mL), dried over Na₂SO₄, filtered and concentrated. N-(3-bromophenyl)-N-methylacetamide (600 mg, yield: 97.9%) was obtained as a yellow oil, which was used directly without purification. ¹H NMR (400 MHz, Dimethylsulfoxide-d₆) δ 7.64 (s, 1H), 7.53 (s, 1H), 7.42-7.32 (m, 2H), 3.40-3.19 (m, 3H), 1.88-1.68 (m, 3H).

### Step 2: N-Methyl-N-(3-(4,4,5,5-tetramethyl-1,3,2-dioxaborolan-2-yl)phenyl)acetamide

To a solution of N-(3-bromophenyl)-N-methyl-acetamide (500 mg, 2.19 mmol) and 4,4,5,5-tetramethyl-2-(4,4,5,5-tetramethyl-1,3,2-dioxaborolan-2-yl)-1,3,2-dioxaborolane (835 mg, 3.29 mmol) in 1,4-dioxane (20 mL) were added AcOK (861 mg, 8.77 mmol) and Pd(dppf)Cl₂ (160 mg, 219 μmol) at 25° C. This mixture was stirred at 100° C. for 12 hr under N₂. The mixture was quenched with H₂O (30 mL) and extracted with EtOAc (20 mL×3). The combined organic layers were washed with brine (50 mL), dried over Na₂SO₄, filtered and concentrated. N-methyl-N-(3-(4,4,5,5-tetramethyl-1,3,2-dioxaborolan-2-yl)phenyl)acetamide (Intermediate 201, 600 mg, yield: 99.5%) was obtained as a black solid, which was used directly without purification. MS: m/z=275.8 [M+H]⁺.

### Intermediate 202: 3-(3-(4-(Chloromethyl)phenyl)-5-(3-methoxypyridin-4-yl)-3H-imidazo[4,5-b]pyridin-2-yl)pyridin-2-amine

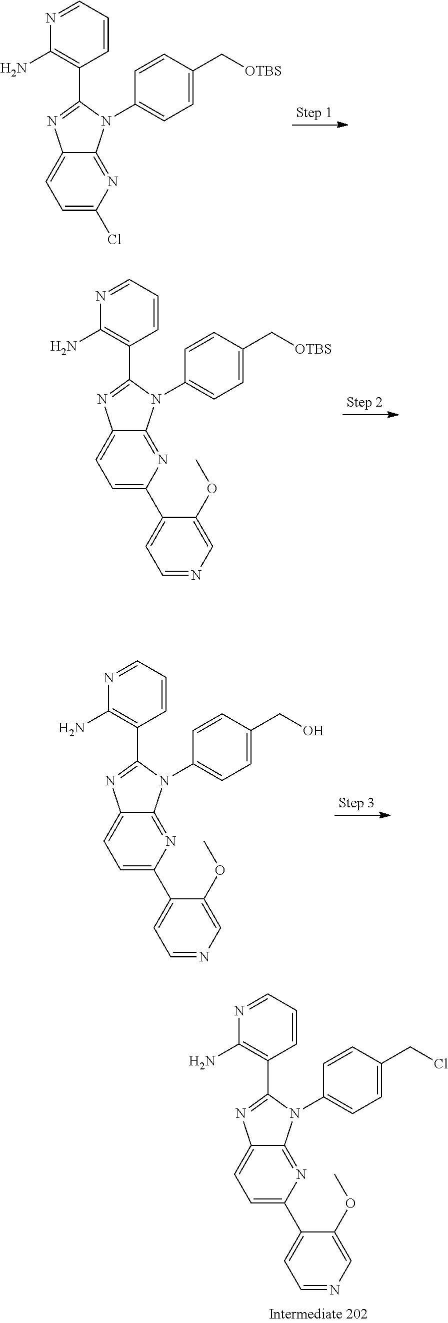

### Step 1: 3-(3-(4-(((Tert-butyldimethylsilyl)oxy)methyl)phenyl)-5-(3-methoxypyridin-4-yl)-3H-imidazo[4,5-b]pyridin-2-yl)pyridin-2-amine

To a solution of 3-(3-(4-(((tert-butyldimethylsilyl)oxy)methyl)phenyl)-5-chloro-3H-imidazo[4,5-b]pyridin-2-yl)pyridin-2-amine (refer to Intermediate 17 for detail procedures, 500 mg, 1.07 mmol) in 1,4-dioxane (5 mL) and H₂O (1 mL) were added Pd(dppf)Cl₂ (78.5 mg, 107 μmol), (3-methoxypyridin-4-yl)boronic acid (213 mg, 1.39 mmol) and Cs₂CO₃ (1.05 g, 3.22 mmol), then degassed and purged with N₂ three times. The mixture was stirred at 100° C. for 12 hr under N₂ atmosphere. Water (50 mL) was added, and the mixture was extracted with EtOAc (20 mL×2). The combined organic layers were dried over anhydrous Na₂SO₄, filtered and concentrated. After purified by silica gel flash chromatography (Eluent of 0˜80% EtOAc in petroleum ether), 3-(3-(4-(((tert-butyldimethylsilyl)oxy)methyl)phenyl)-5-(3-methoxypyridin-4-yl)-3H-imidazo[4,5-b]pyridin-2-yl)pyridin-2-amine (350 mg, yield: 61%) was obtained as a black brown solid. MS: m/z=539.2 [M+H]⁺.

### Step 2: (4-(2-(2-Aminopyridin-3-yl)-5-(3-methoxypyridin-4-yl)-3H-imidazo[4,5-b]pyridin-3-yl)phenyl)methanol

To a solution of 3-(3-(4-(((tert-butyldimethylsilyl)oxy)methyl)phenyl)-5-(3-methoxypyridin-4-yl)-3H-imidazo[4,5-b]pyridin-2-yl)pyridin-2-amine (350 mg, 650 μmol) in THF (4 mL) was added TBAF (1 M in THF, 1.30 mmol). The mixture was stirred at 20° C. for 2 hr. The mixture was diluted with H₂O (50 mL) and extracted with EtOAc (20 mL×2). The combined organic layers were dried over anhydrous Na₂SO₄, filtered and concentrated, (4-(2-(2-aminopyridin-3-yl)-5-(3-methoxypyridin-4-yl)-3H-imidazo[4,5-b]pyridin-3-yl)phenyl)methanol (250 mg, yield. 91%) was obtained as a black brown solid, which was used in the next step directly. MS: m/z=425.2 [M+H]⁺.

### Step 3: 3-(3-(4-(Chloromethyl)phenyl)-5-(3-methoxypyridin-4-yl)-3H-imidazo[4,5-b]pyridin-2-yl)pyridin-2-amine

To a solution of (4-(2-(2-aminopyridin-3-yl)-5-(3-methoxypyridin-4-yl)-3H-imidazo[4,5-b]pyridin-3-yl)phenyl)methanol (250 mg, 589 μmol) in CH₂C12 (3 mL) was added SOCl₂ (210 mg, 1.77 mmol). The mixture was stirred at 25° C. for 2 hr, then concentrated, 3-(3-(4-(chloromethyl)phenyl)-5-(3-methoxypyridin-4-yl)-3H-imidazo[4,5-b]pyridin-2-yl)pyridin-2-amine (Intermediate 202, 160 mg, yield: 61.3%) was obtained as a brown solid, which was used in the next step directly. MS: m/z=443.1 [M+H]⁺.

### Intermediate 203: 3-(3-(4-(Chloromethyl)phenyl)-5-(6-methoxypyridin-3-yl)-3H-imidazo[4,5-b]pyridin-2-yl)pyridin-2-amine

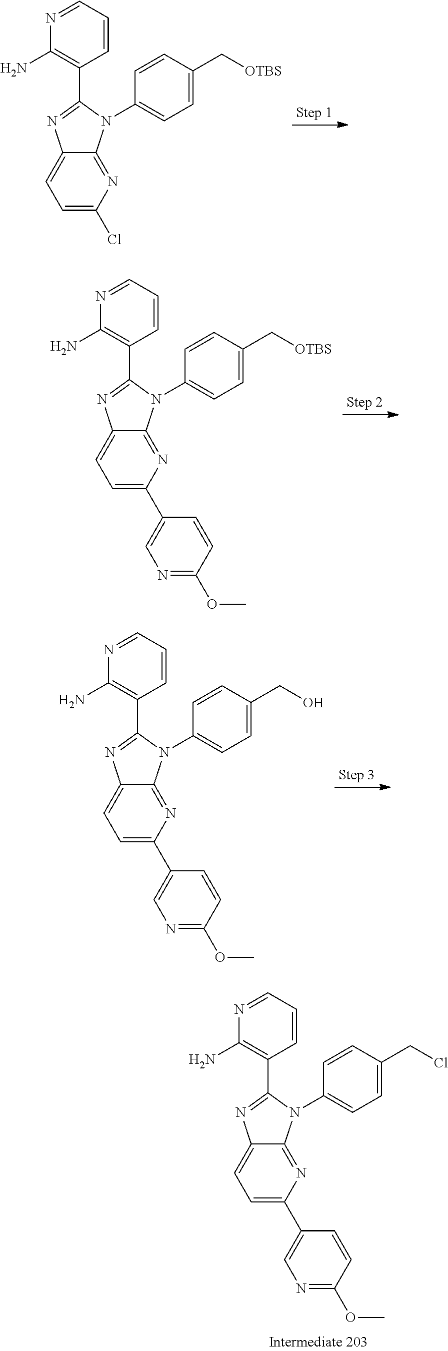

### Step 1: 3-(3-(4-(((tert-Butyldimethylsilyl)oxy)methyl)phenyl)-5-(6-methoxypyridin-3-yl)-3H-imidazo[4,5-b]pyridin-2-yl)pyridin-2-amine

To a solution of 3-(3-(4-(((tert-butyldimethylsilyl)oxy)methyl)phenyl)-5-chloro-3H-imidazo[4,5-b]pyridin-2-yl)pyridin-2-amine (refer to Intermediate 117 for detail procedures, 500 mg, 1.07 mmol) in 1,4-dioxane (5 mL) and H₂O (1 mL) were added Pd(dppf)Cl₂ (78.5 mg, 107 μmol), (6-methoxy-3-pyridyl)boronic acid (213 mg, 1.39 mmol) and Cs₂CO₃ (1.05 g, 3.22 mmol). The mixture was degassed and purged with N₂ three times, and stirred at 100° C. for 2 hr under N₂ atmosphere. The mixture was diluted with H₂O (50 mL) and extracted with EtOAc (30 mL×3). The combined organic layers were washed with brine (50 mL), dried over Na₂SO₄, filtered and concentrated. After purified by silica gel flash chromatography (Eluent of 0˜80% EtOAc in petroleum ether), 3-(3-(4-(((tert-butyldimethylsilyl)oxy)methyl)phenyl)-5-(6-methoxypyridin-3-yl)-3H-imidazo[4,5-b]pyridin-2-yl)pyridin-2-amine (450 mg, yield: 77.9%) was obtained as a brown solid. MS: m/z=539.2 [M+H]⁺.

### Step 2: (4-(2-(2-Aminopyridin-3-yl)-5-(6-methoxypyridin-3-yl)-3H-imidazo[4,5-b]pyridin-3-yl)phenyl)methanol

To a solution of 3-(3-(4-(((tert-butyldimethylsilyl)oxy)methyl)phenyl)-5-(6-methoxypyridin-3-yl)-3H-imidazo[4,5-b]pyridin-2-yl)pyridin-2-amine (440 mg, 817 μmol) in THF (4 mL) was added TBAF (1 M in THF, 1 mL). The mixture was stirred at 25° C. for 2 hr. The mixture was diluted with H₂O (50 mL) and extracted with EtOAc (2×20 mL). The combined organic layers were dried over anhydrous Na₂SO₄, filtered and concentrated. (4-(2-(2-Aminopyridin-3-yl)-5-(6-methoxypyridin-3-yl)-3H-imidazo[4,5-b]pyridin-3-yl)phenyl)methanol (250 mg, yield: 72.1%) was obtained as a brown solid, which was used in the next step directly. MS: m/z=425.2 [M+H]⁺.

### Step 3: 3-(3-(4-(Chloromethyl)phenyl)-5-(6-methoxypyridin-3-yl)-3H-imidazo[4,5-b]pyridin-2-yl)pyridin-2-amine

To a solution of (4-(2(2-aminopyridin-3-yl)-5-(6-methoxypyridin-3-yl)-3H-imidazo[4,5-b]pyridin-3-yl)phenyl)methanol (240 mg, 565 μmol) in CH₂Cl₂ (3 mL) was added SOCl₂ (202 mg, 1.70 mmol). The mixture was stirred at 25° C. for 2 hr and then concentrated. 3-(3-(4-(Chloromethyl)phenyl)-5-(6-methoxypyridin-3-yl)-3H-imidazo[4,5-b]pyridin-2-yl)pyridin-2-amine (Intermediate 203, 250 mg, yield: 99.8%) was obtained as a brown solid, which was used in the next step directly. MS: m/z=443.1 [M+H]⁺.

### Intermediate 204: N-Methyl-N-(1-(3-(4,4,5,5-tetramethyl-1,3,2-dioxaborolan-2-yl)phenyl)piperidin-4-yl)acetamide

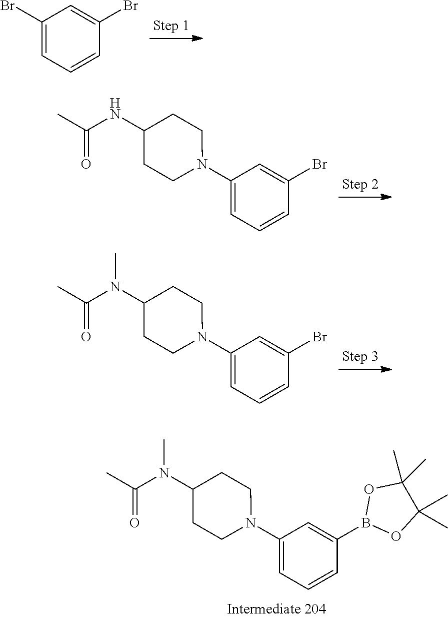

### Step 1: N-(1-(3-Bromophenyl)piperidin-4-yl)acetamide

A mixture of 1,3-dibromobenzene (5 g, 21.2 mmol), N-(piperidin-4-yl)acetamide (3.01 g, 21.2 mmol), Pd₂(dba)₃ (970 mg, 1.06 mmol), BINAP (990 mg, 1.59 mmol) and t-BuONa (2.44 g, 25.4 mmol) in toluene (120 mL) was stirred at 80° C. for 16 hr under N₂. After cooling to 25° C., the mixture was diluted with EtOAc (50 mL) and filtered, concentrated. After purified by silica gel flash chromatography (EtOAc in petroleum ether=50%-100%), N-(1-(3-bromophenyl)piperidin-4-yl)acetamide (4.02 g, yield: 63.8%) was obtained as an off-white solid. MS: m/z=297.0, 299.0 [M+H]⁺. ¹H NMR (400 MHz, Dimethylsulfoxide-d₆) δ 7.81 (d, J=7.6 Hz, 1H), 7.14-7.09 (m, 1H), 7.07-7.04 (m, 1H), 6.93 (dd, J=8.4, 2.0 Hz, 1H), 6.87 (dd, J=7.6, 0.8 Hz, 1H), 3.78-3.62 (m, 3H), 2.86-2.77 (m, 2H), 1.81-1.73 (m, 5H), 1.46-1.35 (m, 2H).

### Step 2: N-(1-(3-Bromophenyl)piperidin-4-yl)-N-methylacetamide

To a solution of N-(1-(3-bromophenyl)piperidin-4-yl)acetamide (2 g, 6.73 mmol) in THF (50 mL) was added NaH (1.08 g, 26.9 mmol, 60% in mineral oil) in portions at 0° C. and stirred at 20° C. for 10 min under N₂, then added the MeI (2.87 g, 20.2 mmol) at 0° C. and stirred at 20° C. for 12 hr. The reaction mixture was slowly quenched with water (50 mL) at 0° C. and extracted with EtOAc (50 mL×2). The combined organic layers were washed with brine (50 mL), dried over anhydrous Na₂SO₄ and filtered, concentrated. After purified by silica gel flash chromatography (EtOAc in petroleum ether=20%-100%), N-(1-(3-bromophenyl)piperidin-4-yl)-N-methylacetamide (1.8 g, yield: 85.9%) was obtained as a brown oil. MS: m/z=310.9, 312.9 [M+H]⁺. ¹H NMR (400 MHz, Dimethylsulfoxide-d₆) δ 7.16-7.08 (m, 1H), 7.07 (d, J=2.4 Hz, 1H), 6.94 (d, J=8.0 Hz, 1H), 6.88 (d, J=7.6 Hz, 1H), 4.51-4.37 (m, 0.5H), 3.83-3.73 (m, 2.5H), 2.88-2.78 (m, 4H), 2.65 (s, 1H), 2.07 (s, 1H), 1.99 (s, 2H), 1.84-1.62 (m, 3H), 1.53-1.47 (m, 1H).

### Step 3: N-Methyl-N-(1-(3-(4,4,5,5-tetramethyl-1,3,2-dioxaborolan-2-yl)phenyl)piperidin-4-yl)acetamide

A mixture of N-(1-(3-bromophenyl)piperidin-4-yl)-N-methylacetamide (1.8 g, 5.78 mmol), 4,4,5,5-tetramethyl-2-(4,4,5,5-tetramethyl-1,3,2-dioxaborolan-2-yl)-1,3,2-dioxaborolane (1.76 g, 6.94 mmol), Pd(dppf)Cl₂ (212 mg, 289 μmol) and K₂CO₃ (2.40 g, 17.4 mmol) in 1,4-dioxane (50 mL) was stirred at 80° C. for 12 hr under N₂. The mixture was diluted with water (50 mL) at 20° C. and extracted with EtOAc (50 mL×2). The combined organic layers were washed with brine (50 mL), dried over anhydrous Na₂SO₄, filtered, and concentrated. N-Methyl-N-(1-(3-(4,4,5,5-tetramethyl-1,3,2-dioxaborolan-2-yl)phenyl)piperidin-4-yl)acetamide (Intermediate 204, 2 g, 5.58 mmol, yield: 96.5%) was obtained as a gray solid, which was used directly into the next step. MS: m/z=359.0 [M+H]+. ¹H NMR (400 MHz, Chloroform-d) δ 7.39 (s, 1H), 7.36-7.26 (m, 2H), 7.05 (d, J=6.4 Hz, 1H), 4.69-4.61 (m, 1H), 3.84-3.74 (m, 2H), 2.90-2.75 (m, 5H), 2.16 (s, 1H), 2.11 (s, 2H), 2.05-2.01 (m, 1H), 1.79-1.68 (m, 3H), 1.34 (s, 12H).

### Intermediate 205: 3-(3-(4-(Chloromethyl)phenyl)-5-(4-fluoro-2-(trifluoromethyl)phenyl)-3H-imidazo[4,5-b]pyridin-2-yl)pyridin-2-amine

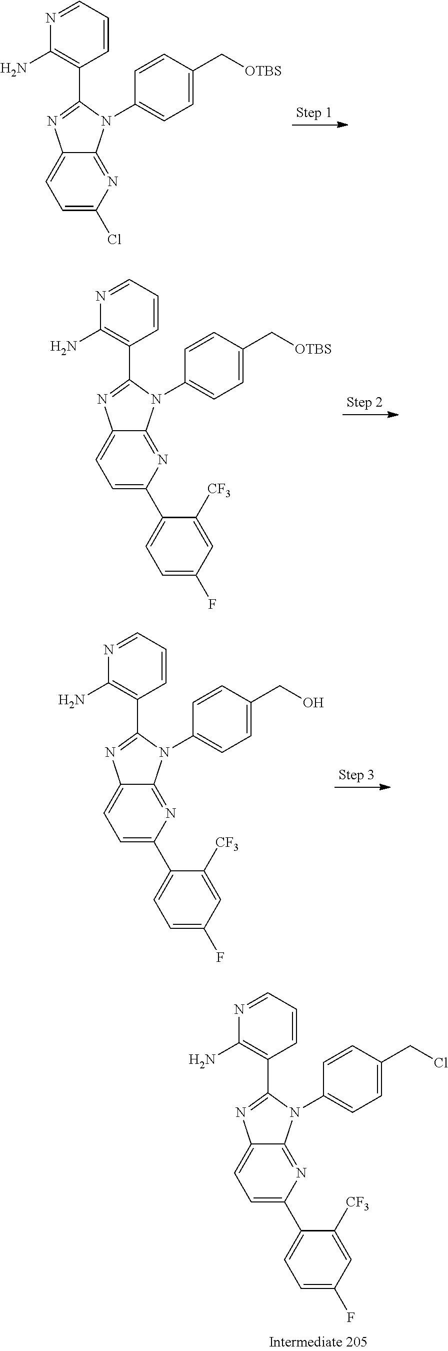

### Step 1: 3-(3-(4-(((Tert-butyldimethylsilyl)oxy)methyl)phenyl)-5-(4-fluoro-2-(trifluoromethyl)phenyl)-3H-imidazo[4,5-b]pyridin-2-yl)pyridin-2-amine

To a mixture of 3-(3-(4-(((tert-butyldimethylsilyl)oxy)methyl)phenyl)-5-chloro-3H-imidazo[4,5-b]pyridin-2-yl)pyridin-2-amine (refer to Intermediate 117 for detail procedures, 400 mg, 858.3 μmol) and (4-fluoro-2-(trifluoromethyl)phenyl)boronic acid (196.3 mg, 944.1 μmol) in 1,4-dioxane (5 mL) and H₂O (1 mL) were added Cs₂CO₃ (838.9 mg, 2.6 mmol) and Pd(dppf)Cl₂ (62.8 mg, 85.8 μmol), the mixture was stirred at 100° C. for 2 hr under N₂. H₂O (50 mL) was added and extracted with EtOAc (100 mL×2). The combined organic layers were washed with brine (100 mL), dried over anhydrous Na₂SO₄, filtered and concentrated. After purified by silica gel flash chromatography ((ethyl acetate/EtOH=3/1) in petroleum ether=0 to 80%), 3-(3-(4-(((tert-butyldimethylsilyl)oxy)methyl)phenyl)-5-(4-fluoro-2-(trifluoromethyl)phenyl)-3H-imidazo[4,5-b]pyridin-2-yl)pyridin-2-amine (400 mg, 673.8 μmol, yield: 78.5%) was obtained as a brown solid. MS: m/z=594.3 [M+H]⁺. ¹H NMR (400 MHz, Dimethylsulfoxide-d₆) δ 8.30 (d, J=8.0 Hz, 1H), 8.03-7.96 (m, 1H), 7.74 (d, J=9.2 Hz, 1H), 7.62 (d, J=7.2 Hz, 2H), 7.51 (d, J=8.0 Hz, 1H), 7.46-7.34 (m, 4H), 7.20 (dd, J=7.6, 1.6 Hz, 1H), 7.07-6.96 (m, 2H), 6.38 (dd, J=7.6, 4.8 Hz, 1H), 4.77 (s, 2H), 0.90 (s, 9H), 0.08 (s, 6H).

### Step 2: (4-(2-(2-Aminopyridin-3-yl)-5-(4-fluoro-2-(trifluoromethyl)phenyl)-3H-imidazo[4,5-b]pyridin-3-yl)phenyl)methanol

To a mixture of 3-(3-(4-(((tert-butyldimethylsilyl)oxy)methyl)phenyl)-5-(4-fluoro-2-(trifluoromethyl)phenyl)-3H-imidazo[4,5-b]pyridin-2-yl)pyridin-2-amine (350.0 mg, 589.5 μmol) in THF (2 mL) was added TBAF (1 M in THF, 884.3 μL). The mixture was stirred at 20° C. for 1 hr. The mixture was diluted with H₂O (50 mL) and extracted with EtOAc (50 mL×2). The combined organic layers were washed with brine (50 mL), dried over anhydrous Na₂SO₄, filtered and concentrated. (4-(2-(2-Aminopyridin-3-yl)-5-(4-fluoro-2-(trifluoromethyl)phenyl)-3H-imidazo[4,5-b]pyridin-3-yl)phenyl)methanol (280 mg, 584.0 μmol, yield: 99.1%) was obtained as a brown solid. MS: m/z=480.2 [M+H]⁺.

### Step 3: 3-(3-(4-(Chloromethyl)phenyl)-5-(4-fluoro-2-(trifluoromethyl)phenyl)-3H-imidazo[4,5-b]pyridin-2-yl)pyridin-2-amine

To a mixture of (4-(2-(2-aminopyridin-3-yl)-5-(4-fluoro-2-(trifluoromethyl)phenyl)-3H-imidazo[4,5-b]pyridin-3-yl)phenyl)methanol (270.0 mg, 563.2 μmol) in CH₂Cl₂ (2 mL) was added SOCl₂ (201.0 mg, 1.69 mmol). The mixture was stirred at 40° C. for 1 hr and then concentrated under reduced pressure. 3-(3-(4-(Chloromethyl)phenyl)-5-(4-fluoro-2-(trifluoromethyl)phenyl)-3H-imidazo[4,5-b]pyridin-2-yl)pyridin-2-amine (Intermediate 205, 280 mg, 562.4 μmol, yield: 99.9%) was obtained as a yellow solid. MS: m/z=498.1 [M+H]⁺.

### Intermediate 206: 3-(3-(4-(Chloromethyl)phenyl)-5-(p-tolyl)-3H-imidazo[4,5-b]pyridin-2-yl)pyridin-2-amine

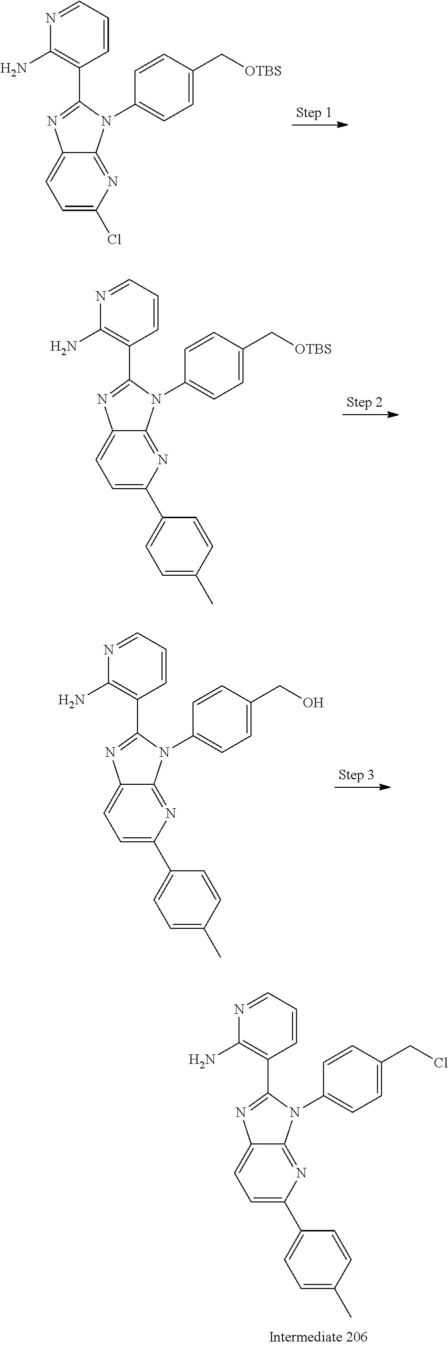

### Step 1: 3-(3-(4-(((Tert-butyldimethylsilyl)oxy)methyl)phenyl)-5-(p-tolyl)-3H-imidazo[4,5-b]pyridin-2-yl)pyridin-2-amine

To a solution of 3-(3-(4-(((tert-butyldimethylsilyl)oxy)methyl)phenyl)-5-chloro-3H-imidazo[4,5-b]pyridin-2-yl)pyridin-2-amine (refer to Intermediate 117 for detail procedures, 400 mg, 858 μmol) in 1,4-dioxane (5 mL) and H₂O (1 mL) were added p-tolylboronic acid (163 mg, 1.2 mmol), Pd(dppf)Cl₂ (62.8 mg, 85.8 μmol), Cs₂CO₃ (839 mg, 2.6 mmol). Water (30 mL) was added and extracted with EtOAc (50 mL×3), the combined organic layers were washed with brine (50 mL×2), dried over anhydrous Na₂SO₄, filtered and concentrated under reduced pressure. The mixture was stirred at 100° C. for 1 hr under N₂. After purified by silica gel flash chromatography (Eluent of 0%˜36% EtOAc in petroleum ether), 3-(3-(4-(((tert-butyldimethylsilyl)oxy)methyl)phenyl)-5-(p-tolyl)-3H-imidazo[4,5-b]pyridin-2-yl)pyridin-2-amine (258 mg, yield: 58%) was obtained as a red solid. ¹H NMR (400 MHz, Chloroform-d) δ 8.12 (d, J=8.4 Hz, 1H), 8.08 (d, J=4.4 Hz, 1H), 7.94 (d, J=8.0 Hz, 2H), 7.80 (d, J=8.4 Hz, 1H), 7.55-7.48 (m, 2H), 7.46-7.40 (m, 2H), 7.31-7.21 (m, 3H), 7.16 (d, J=7.6 Hz, 1H), 6.70 (br s, 2H), 6.40 (dd, J=7.2, 4.4 Hz, 1H), 4.88 (s, 2H), 2.41 (s, 3H), 1.00 (s, 9H), 0.18 (s, 6H).

### Step 2: (4-(2-(2-Aminopyridin-3-yl)-5-(p-tolyl)-3H-imidazo[4,5-b]pyridin-3-yl)phenyl)methanol

To a solution of 3-(3-(4-(((tert-butyldimethylsilyl)oxy)methyl)phenyl)-5-(p-tolyl)-3H-imidazo[4,5-b]pyridin-2-yl)pyridin-2-amine (258 mg, 494.5 μmol) in THF (4 mL) was added TBAF (1 M in THF, 791 μL). The mixture was stirred at 25° C. for 1 hr. The reaction mixture was quenched with H₂O (10 mL) and extracted with EtOAc (10 mL×2). The organic layers were washed with brine (15 mL), dried over Na₂SO₄, filtered and concentrated under reduced pressure. (4-(2-(2-Aminopyridin-3-yl)-5-(p-tolyl)-3H-imidazo[4,5-b]pyridin-3-yl)phenyl)methanol (120 mg, yield: 60%) was obtained as a red solid, which was used in the next step without further purification. MS: m/z=408.3 [M+H]⁺. ¹H NMR (400 MHz, Chloroform-d) δ 8.00 (d, J=8.8 Hz, 1H), 7.96 (dd, J=4.8, 1.6 Hz, 1H), 7.81 (d, J=8.0 Hz, 2H), 7.67 (d, J=8.4 Hz, 1H), 7.46-7.41 (m, 2H), 7.37-7.32 (m, 2H), 7.13 (d, J=8.0 Hz, 2H), 7.02 (dd, J=7.6, 1.6 Hz, 1H), 6.49 (br s, 2H), 6.29 (dd, J=8, 4.8 Hz, 1H), 4.73 (s, 2H), 3.28-3.18 (m, 1H), 2.28 (s, 3H).

### Step 3: 3-(3-(4-(Chloromethyl)phenyl)-5-(p-tolyl)-3H-imidazo[4,5-b]pyridin-2-yl)pyridin-2-amine

To a solution of (4-(2-(2-aminopyridin-3-yl)-5-(p-tolyl)-3H-imidazo[4,5-b]pyridin-3-yl)phenyl)methanol (120 mg, 294.5 μmol) in CH₂Cl₂ (2 mL) was added SOCl₂ (21.4 μL, 295 μmol). The mixture was stirred at 25° C. for 1 hr. The reaction was concentrated under reduced pressure to give 3-(3-(4-(chloromethyl)phenyl)-5-(p-tolyl)-3H-imidazo[4,5-b]pyridin-2-yl)pyridin-2-amine (Intermediate 206, 125 mg, yield: 100%) as a brown solid, which was used in the next step without further purification. MS: m/z=426.3 [M+H]⁺

### Intermediate 207: 3-(3-(4-(Chloromethyl)phenyl)-5-(3-(trifluoromethyl)phenyl)-3H-imidazo[4,5-b]pyridin-2-yl)pyridin-2-amine

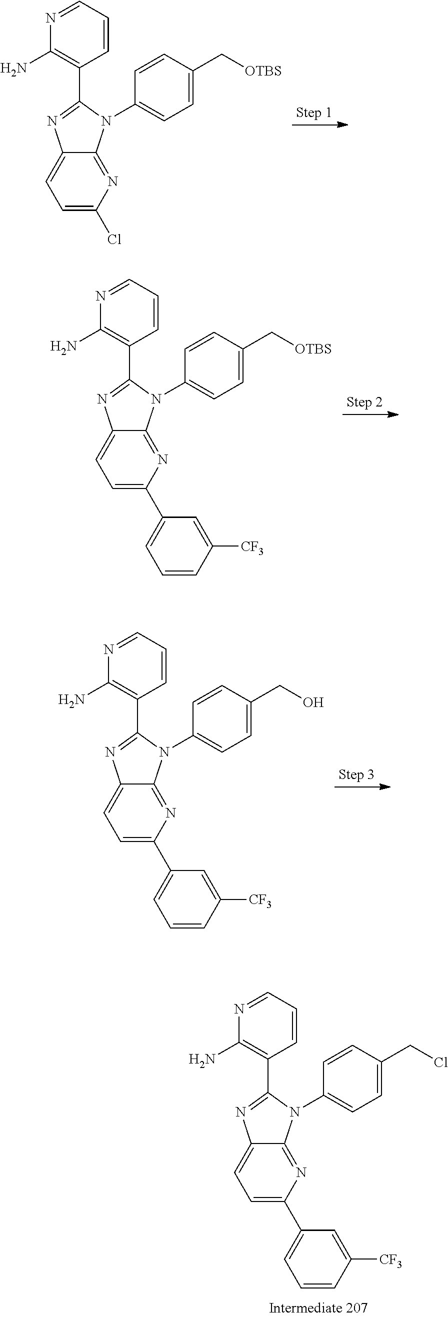

### Step 1: 3-(3-(4-(((Tert-butyldimethylsilyl)oxy)methyl)phenyl)-5-(3-(trifluoromethyl)phenyl)-3H-imidazo[4,5-b]pyridin-2-yl)pyridin-2-amine

To a solution of 3-(3-(4-(((tert-butyldimethylsilyl)oxy)methyl)phenyl)-5-chloro-3H-imidazo[4,5-b]pyridin-2-yl)pyridin-2-amine (refer to Intermediate 117 for detail procedures, 400 mg, 858.3 μmol) in 1,4-dioxane (5 mL), H₂O (1 mL) were added (3-(trifluoromethyl)phenyl)boronic acid (228.2 mg, 1.2 mmol), Pd(dppf)Cl₂ (62.8 mg, 85.8 μmol), and Cs₂CO₃ (838.9 mg, 2.6 mmol). The mixture was stirred at 100° C. for 1 hr under N₂. Water (30 mL) was added and extracted with EtOAc (50 mL×3), the combined organic layers were washed with brine (50 mL×2), dried over anhydrous Na₂SO₄, filtered, and concentrated under reduced pressure. After purified by silica gel flash chromatography (ethyl acetate in petroleum ether=0-36%), 3-(3-(4-(((tert-butyldimethylsilyl)oxy)methyl)phenyl)-5-(3-(trifluoromethyl)phenyl)-3H-imidazo[4,5-b]pyridin-2-yl)pyridin-2-amine (333 mg, yield: 67.4%) was obtained as a red solid. MS: m/z=576.3 [M+H]^{+ 1}H NMR (400 MHz, Chloroform-d) δ 8.10 (s, 1H), 8.05 (d, J=7.8 Hz, 1H), 8.00 (d, J=8.4 Hz, 1H), 7.91 (dd, J=4.8, 1.6 Hz, 1H), 7.67 (d, J=8.4 Hz, 1H), 7.43-7.48 (m, 1H), 7.40 (d, J=7.6 Hz, 1H), 7.35 (d, J=8.4 Hz, 2H), 7.24-7.22 (m, 1H), 7.10 (s, 1H), 7.00 (dd, J=8.0, 1.6 Hz, 1H), 6.59 (br s, 2H), 6.23 (dd, J=7.6, 4.8 Hz, 1H), 4.71 (s, 2H), 0.82 (s, 9H), 0.11 (s, 6H). ¹⁹F NMR (400 MHz, Chloroform-d₆) δ −62.639.

### Step 2: (4-(2-(2-Aminopyridin-3-yl)-5-(3-(trifluoromethyl)phenyl)-3H-imidazo[4,5-b]pyridin-3-yl)phenyl)methanol

To a solution of 3-(3-(4-(((tert-butyldimethylsilyl)oxy)methyl)phenyl)-5-(3-(trifluoromethyl)phenyl)-3H-imidazo[4,5-b]pyridin-2-yl)pyridin-2-amine (333 mg, 578.4 μmol) in THF (4 mL) was added TBAF (1 M in THF, 925.5 μL). The mixture was stirred at 25° C. for 1 hr under N₂. The mixture was quenched with H₂O (20 mL) and extracted with EtOAc (10 mL×2). The organic layer were washed with brine (15 mL), dried over Na₂SO₄, then concentrated. (4-(2-(2-Aminopyridin-3-yl)-5-(3-(trifluoromethyl)phenyl)-3H-imidazo[4,5-b]pyridin-3-yl)phenyl)methanol (266.9 mg, yield: 100%) was obtained as a red solid, which was used in the next step without further purification. MS: m/z=462.2 [M+H]⁺. ¹H NMR (400 MHz, Chloroform-d) δ 8.14 (s, 1H), 8.10 (d, J=8 Hz, 1H), 8.05 (d, J=8.4 Hz, 1H), 7.97 (dd, J=4.8, 1.6 Hz, 1H), 7.72 (d, J=8.0 Hz, 1H), 7.53-7.49 (m, 4H), 7.32 (d, J=8.0 Hz, 2H), 7.03 (dd, J=8.0, 2.0 Hz, 1H), 6.53 (br s, 2H), 6.29 (dd, 1=7.6, 4.8 Hz, 1H), 4.75 (s, 2H), 2.27-2.47 (m, 1H). ¹⁹F NMR (400 MHz, Chloroform-do) 6-62.599.

### Step 3: 3-(3-(4-(Chloromethyl)phenyl)-5-(3-(trifluoromethyl)phenyl)-3H-imidazo[4,5-b]pyridin-2-yl)pyridin-2-amine

To a solution of (4-(2-(2-aminopyridin-3-yl)-5-(3-(trifluoromethyl)phenyl)-3H-imidazo[4,5-b]pyridin-3-yl)phenyl)methanol (266.9 mg, 578.4 μmol) in CH₂Cl₂ (3.5 mL) was added SOCl₂ (206.5 mg, 1.7 mmol). The mixture was stirred at 25° C. for 1 hr under N₂. The reaction mixture was concentrated. 3-(3-(4-(Chloromethyl)phenyl)-5-(3-(trifluoromethyl)phenyl)-3H-imidazo[4,5-b]pyridin-2-yl)pyridin-2-amine (intermediate 207, 277.6 mg, yield: 100′%) was obtained as a brown solid, which was used in the next step without further purification. MS: m/z=480.2 [M+H]⁺. H NMR (400 MHz, Dimethylsulfoxide-d₆) δ 8.52 (br s, 1H), 8.42 (d, J=8.4 Hz, 1H), 8.34-8.40 (m, 2H), 8.22 (d, 1=8.4 Hz, 1H), 8.17 (dd, J=6.0, 1.6 Hz, 1H), 7.93 (dd, J=7.6, 1.6 Hz, 1H), 7.71-7.81 (m, 2H), 7.64-7.60 (m, 4H), 6.92 (t, J=7.2 Hz, 1H), 4.88 (s, 2H). ¹⁹F NMR (400 MHz, Dimethylsulfoxide-d₆) δ −61.094.

### Intermediate 208: 3-(1-(4-(Chloromethyl)phenyl)-1H-benzo[d]imidazol-2-yl)pyridin-2-amine

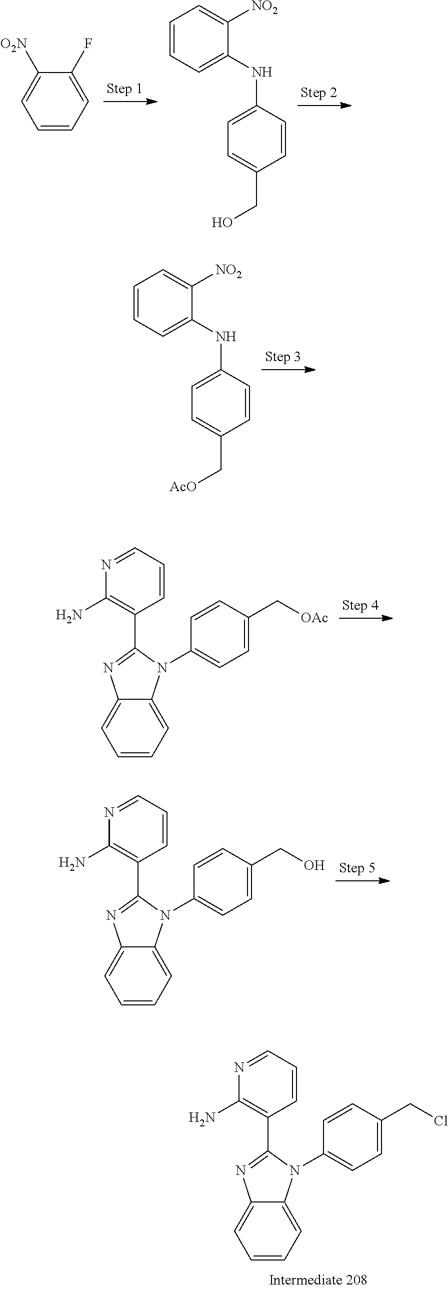

### Step 1: (4-((2-Nitrophenyl)amino)phenyl)methanol

To a solution of 1-fluoro-2-nitrobenzene (5 g, 35.4 mmol) and (4-aminophenyl)methanol (4.36 g, 35.4 mmol) in DMSO (50 mL) was added DIPEA (13.7 g, 106 mmol) at 20° C. The reaction mixture was stirred at 100° C. for 12 hr. The mixture was diluted with H₂O (100 mL) and the mixture was extracted with EtOAc (100 mL×3). The combined organic layers were washed with brine (100 mL×2), dried over anhydrous Na₂SO₄, filtered and concentrated under reduced pressure. (4-((2-Nitrophenyl)amino)phenyl)methanol (8.66 g, crude) was obtained as brown oil, which was used directly into the next step.

### Step 2: 4-((2-Nitrophenyl)amino)benzyl acetate

To a solution of (4-((2-nitrophenyl)amino)phenyl)methanol (2.8 g, 11.5 mmol) in CH₂Cl₂ (30 mL) were added DMAP (140 mg, 1.15 mmol), TEA (3.48 g, 34.4 mmol) and Ac₂O (1.76 g, 17.2 mmol). The mixture was stirred at 20° C. for 1 hr. H₂O (30 mL) was added and the mixture was extracted with CH₂Cl₂ (30 mL×3). The combined organic layers were washed with brine (30 mL×2), dried over anhydrous Na₂SO₄, filtered and concentrated under reduced pressure. After purified by silica gel flash chromatography (EtOAc in petroleum ether=0-20%), 4-((2-nitrophenyl)amino)benzyl acetate (1.97 g, yield: 60%) was obtained as a brown oil. ¹H NMR (400 MHz, Dimethylsulfoxide-d₆) δ 9.34 (s, 1H), 8.11 (dd, J=8.4, 1.6 Hz, 1H), 7.54-7.48 (m, 1H), 7.42-7.38 (m, 2H), 7.33-7.29 (m, 2H), 7.23 (d, J=8.0 Hz, 1H), 6.92-6.87 (m, 1H), 5.06 (s, 2H), 2.07 (s, 3H).

### Step 3: 4-(2-(2-Aminopyridin-3-yl)-1H-benzo[d]imidazol-1-yl)benzyl acetate

To a solution of 4-((2-nitrophenyl)amino)benzyl acetate (950 mg, 3.32 mmol) in DMSO (30 mL) were added Na₂S₂O₄ (1.73 g, 9.96 mmol) and 2-aminopyridine-3-carbaldehyde (446 mg, 3.65 mmol). The mixture was stirred at 100° C. for 12 hr. Brine (30 mL) was added to the mixture, and it was extracted with CH₂Cl₂ (30 mL×2). The combined organic phase was washed with brine (30 mL), dried over anhydrous Na₂SO₄, filtered and concentrated. After purified by silica gel flash chromatography (EtOAc in petroleum ether=55-100%), 4-(2-(2-aminopyridin-3-yl)-1H-benzo[d]imidazol-1-yl)benzyl acetate (230 mg, yield: 19.3%) was obtained as an off-white solid. MS: m/z=359.2 [M+H]⁺. ¹H NMR (400 MHz, Dimethylsulfoxide-d₆) δ 7.97 (dd, J=4.4, 1.6 Hz, 1H), 7.80 (d, J=7.6 Hz, 1H), 7.56 (d, J=8.4 Hz, 2H), 7.44 (d, J=8.4 Hz, 2H), 7.36-7.26 (m, 2H), 7.19 (d, J=7.6 Hz, 1H), 7.14 (dd, J=1.6, 7.6 Hz, 1H), 6.98 (s, 2H), 6.39 (dd, J=4.8, 7.6 Hz, 1H), 5.17 (s, 2H), 2.11 (s, 3H).

### Step 4: (4-(2-(2-Aminopyridin-3-yl)-1H-benzo[d]imidazol-1-yl)phenyl)methanol

To a mixture of 4-(2-(2-aminopyridin-3-yl)-1H-benzo[d]imidazol-1-yl)benzyl acetate (220 mg, 614 μmol) in THF (3 mL), MeOH (3 mL) and H₂O (1 mL) was added K₂CO₃ (255 mg, 1.84 mmol) at 20° C. The reaction mixture was stirred at 20° C. for 0.5 hr. Then it was diluted with water (10 mL) and extracted with EtOAc (10 mL×2). The combined organic layers were washed with brine (10 mL×2), dried over anhydrous Na₂SO₄, filtered and concentrated. (4-(2-(2-Aminopyridin-3-yl)-1H-benzo[d]imidazol-1-yl)phenyl)methanol (180 mg, yield: 92.7%) was obtained as a yellow solid, which was used directly into the next step. MS: m/z=317.2 [M+H]⁺.

### Step 5: 3-(1-(4-(Chloromethyl)phenyl)-1H-benzo[d]imidazol-2-yl)pyridin-2-amine

To a solution of (4-(2-(2-aminopyridin-3-yl)-1H-benzo[d]imidazol-1-yl)phenyl)methanol (170 mg, 537 μmol) in CH₂Cl₂ (5 mL) was added SOCl₂ (192 mg, 1.61 mmol). The reaction mixture was stirred at 20° C. for 2 hr. The reaction mixture was concentrated. 3-(1-(4-(Chloromethyl)phenyl)-1H-benzo[d]imidazol-2-yl)pyridin-2-amine (Intermediate 208, 180 mg, crude) was obtained as a yellow solid, which was used directly into the next step. MS: m/z=335.1 [M+H]⁺.

### Intermediate 209: 3-(1-(4-(Chloromethyl)phenyl)-1H-imidazo[4,5-b]pyridin-2-yl)pyridin-2-amine

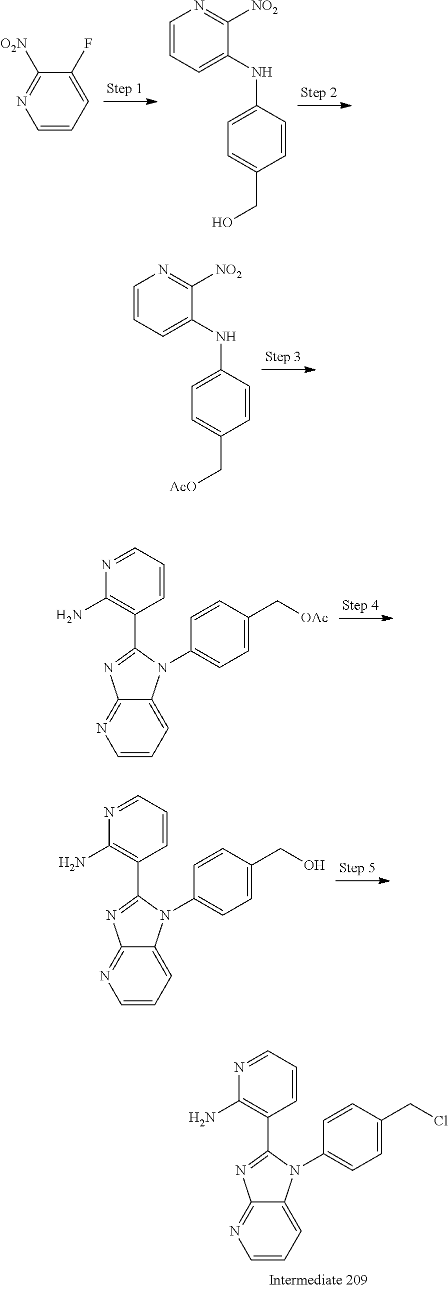

### Step 1: (4-((2-Nitropyridin-3-yl)amino)phenyl)methanol

To a solution of 3-fluoro-2-nitro-pyridine (5 g, 35.2 mmol) and (4-aminophenyl)methanol (4.33 g, 35.2 mmol) in DMF (50 mL) was added TEA (10.7 g, 106 mmol) at 20° C. The reaction mixture was stirred at 100° C. for 12 hr. H₂O (100 mL) was added, and the mixture was extracted with EtOAc (100 mL×3). The combined organic layers were washed with brine (100 mL×2), dried over anhydrous Na₂SO₄, filtered, and concentrated under reduced pressure. (4-((2-Nitropyridin-3-yl)amino)phenyl)methanol (8.63 g, crude) was obtained as brown oil, which was used directly into the next step. MS: m/z=246.0 [M+H]⁺.

### Step 2: 4-((2-Nitropyridin-3-yl)amino)benzyl acetate

To a solution of (4-((2-nitropyridin-3-yl)amino)phenyl)methanol (2.8 g, 11.4 mmol) in CH₂Cl₂ (30 mL) were added DMAP (139 mg, 1.14 mmol), TEA (3.47 g, 34.3 mmol), and Ac₂O (1.75 g, 17.1 mmol). The mixture was stirred at 20° C. for 1 hr. H₂O (30 mL) was added, and the resulting mixture was extracted with CH₂Cl₂ (30 mL×3). The combined organic layers were washed with brine (30 mL×2), dried over anhydrous Na₂SO₄, filtered and concentrated under reduced pressure. After purified by silica gel flash chromatography (EtOAc in petroleum ether=0-50%), 4-((2-nitropyridin-3-yl)amino)benzyl acetate (2.23 g, yield: 68%) was obtained as an orange solid. MS: m/z=288.0 [M+H]⁺. ¹H NMR (400 MHz, Dimethylsulfoxide-d₆) δ 9.13 (s, 1H), 7.99 (dd, J=4.0, 1.2 Hz, 1H), 7.77-7.72 (m, 1H), 7.57 (dd, J=8.4, 4.0 Hz, 1H), 7.40 (d, J=8.4 Hz, 2H), 7.29 (d, J=8.4 Hz, 2H), 5.06 (s, 2H), 2.07 (s, 3H).

### Step 3: 4-(2-(2-Aminopyridin-3-yl)-1H-imidazo[4,5-b]pyridin-1-yl)benzyl acetate

To a solution of 4-((2-nitropyridin-3-yl)amino)benzyl acetate (1.10 g, 3.84 mmol) in DMSO (30 mL) were added Na₂S₂O₄ (2.01 g, 11.5 mmol) and 2-aminopyridine-3-carbaldehyde (516 mg, 4.23 mmol). The mixture was stirred at 100° C. for 12 hr. Brine (30 mL) was added to the mixture and the mixture was extracted with CH₂Cl₂ in MeOH (10:1) (30 mL×2). The combined organic layers were washed with brine (30 mL), dried over anhydrous Na₂SO₄, filtered and concentrated. After purified by silica gel flash chromatography (MeOH in CH₂Cl₂=0-30%), 4-(2-(2-aminopyridin-3-yl)-1H-imidazo[4,5-b]pyridin-1-yl)benzyl acetate (300 mg, yield: 21.7%) was obtained as brown oil. MS: m/z=360.2 [M+H]⁺.

### Step 4: (4-(2-(2-Aminopyridin-3-yl)-1H-imidazo[4,5-b]pyridin-1-yl)phenyl)methanol

To a mixture of 4-(2-(2-aminopyridin-3-yl)-1H-imidazo[4,5-b]pyridin-1-yl)benzyl acetate (290 mg, 807 μmol) in THF (3 mL), MeOH (3 mL) and H₂O (1 mL) was added K₂CO₃ (335 mg, 2.42 mmol) at 20° C. The reaction mixture was stirred at 20° C. for 0.5 hr. The resulting mixture was diluted with brine (10 mL) and extracted with EtOAc (10 mL×2). The combined organic layers were washed with brine (10 mL×2), dried over anhydrous Na₂SO₄, filtered and concentrated. (4-(2-(2-Aminopyridin-3-yl)-1H-imidazo[4,5-b]pyridin-1-yl)phenyl)methanol (110 mg, yield: 43%) was obtained as a brown solid, which was used directly into the next step. MS: m/z=318.1 [M+H]⁺.

### Step 5: 3-(1-(4-(Chloromethyl)phenyl)-1H-imidazo[4,5-b]pyridin-2-yl)pyridin-2-amine

To a solution of (4-(2-(2-aminopyridin-3-yl)-1H-imidazo[4,5-b]pyridin-1-yl)phenyl)methanol (100 mg, 315 μmol) in CH₂Cl₂ (3 mL) was added SOCl₂ (112 mg, 945 μmol). The reaction mixture was stirred at 20° C. for 1 hr. The reaction mixture was concentrated. 3-(1-(4-(chloromethyl)phenyl)-1H-imidazo[4,5-b]pyridin-2-yl)pyridin-2-amine (Intermediate 209, 106 mg, crude) was obtained as a yellow solid, which was used directly into the next step. MS: m/z=336.0 [M+H]⁺.

### Intermediate 210: 3-(3-(4-(Chloromethyl)phenyl)-5-(difluoromethoxy)-3H-imidazo[4,5-b]pyridin-2-yl)pyridin-2-amine

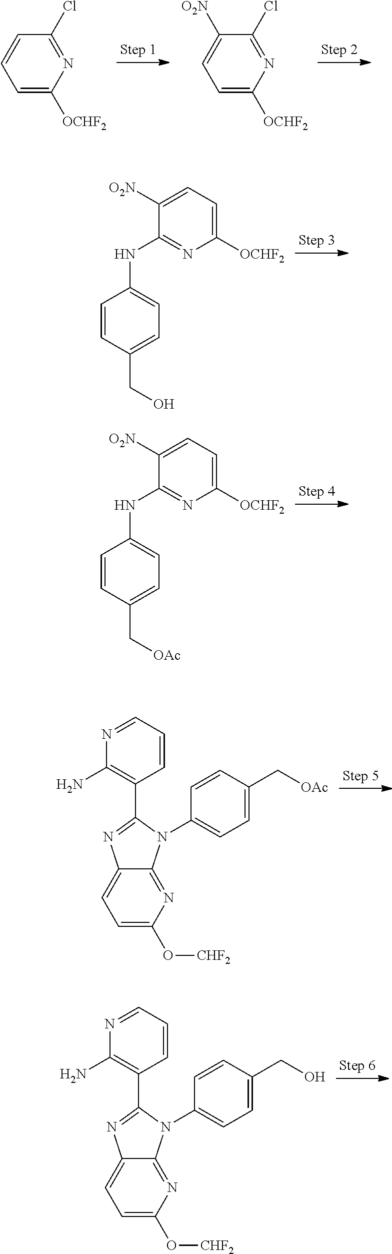

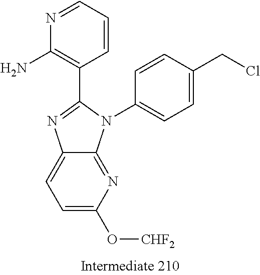 [SMILES: Nc1ncccc1-c1nc2ccc(OC(F)F)nc2n1-c1ccc(CCl)cc1]

### Step 1: 2-Chloro-6-(difluoromethoxy)-3-nitropyridine

To a solution of 2-chloro-6-(difluoromethoxy)pyridine (1.4 g, 7.80 mmol) in H₂SO₄ (14 mL) was added dropwise HNO₃ (9.80 g, 156 mmol, 7 mL) slowly at 0° C. for 1 hr. The reaction mixture was stirred at 40° C. for 12 hr. The reaction mixture was poured into ice water (60 mL) and extracted with EtOAc (60 mL×3). The combined organic layers were washed with brine (60 mL×3), dried over anhydrous Na₂SO₄, filtered and concentrated. After purified by silica gel flash chromatography (EtOAc in petroleum ether=0-20%), 2-chloro-6-(difluoromethoxy)-3-nitropyridine (0.9 g, yield: 51%) was obtained as a yellow oil. ¹H NMR (400 MHz, Chloroform-d) δ 9.11 (s, 1H), 7.55 (t, J=69.6 Hz, 1H). ¹⁹F NMR (400 MHz, Chloroform-d) 6-89.627.

### Step 2: (4-((6-(Difluoromethoxy)-3-nitropyridin-2-yl)amino)phenyl)methanol

To a solution of 2-chloro-6-(difluoromethoxy)-3-nitropyridine (0.9 g, 4.01 mmol) in 1,4-dioxane (15 mL) were added (4-aminophenyl)methanol (543 mg, 4.41 mmol) and DIEA (1.55 g, 12.0 mmol) at 20° C. The reaction mixture was stirred at 80° C. for 2 hr. The reaction mixture was concentrated to give (4-((6-(difluoromethoxy)-3-nitropyridin-2-yl)amino)phenyl)methanol (1.25 g, crude) as a brown oil, which was used directly into the next step.

### Step 3: 4-((6-(Difluoromethoxy)-3-nitropyridin-2-yl)amino)benzyl acetate

To a solution of (4-((6-(difluoromethoxy)-3-nitropyridin-2-yl)amino)phenyl)methanol (0.65 g, 2.09 mmol) in CH₂Cl₂ (6 mL) were added Ac₂O (320 mg, 3.13 mmol), DMAP (25.5 mg, 209 μmol) and TEA (634 mg, 6.27 mmol). The mixture was stirred at 20° C. for 12 hr. The reaction was diluted with H₂O (10 mL) and extracted with CH₂Cl₂ (10 mL×3). The combined organic layers were washed with brine (10 mL×2), dried over anhydrous Na₂SO₄, filtered and concentrated under reduced pressure. After purified by silica gel flash chromatography (Eluent of 10˜20% EtOAc in petroleum ether), 4-((6-(difluoromethoxy)-3-nitropyridin-2-yl)amino)benzyl acetate (290 mg, yield: 39% for 2 steps) was obtained as an orange solid. ¹H NMR (400 MHz, Dimethylsulfoxide-d₆) δ 10.32 (s, 1H), 8.64 (d, J=9.2 Hz, 1H), 7.74-7.53 (m, 3H), 7.41-7.36 (m, 2H), 6.59 (d, J=8.8 Hz, 1H), 5.07 (s, 2H), 2.07 (s, 3H). ¹⁹F NMR (400 MHz, Dimethylsulfoxide-d₆) δ −88.486.

### Step 4: 4-(2-(2-Aminopyridin-3-yl)-5-(difluoromethoxy)-3H-imidazo[4,5-b]pyridin-3-yl)benzyl acetate

To a solution of 4-((6-(difluoromethoxy)-3-nitropyridin-2-yl)amino)benzyl acetate (290 mg, 821 μmol) in DMSO (10 mL) were added Na₂S₂O₄ (429 mg, 2.46 mmol) and 2-aminonicotinaldehyde (110 mg, 903 μmol). The mixture was stirred at 100° C. for 12 hr. Aqueous NaHCO₃ (30 mL) was added and the mixture was extracted with CH₂Cl₂ (30 mL×2). The combined organic layers were washed with brine (30 mL), dried over anhydrous Na₂SO₄, filtered and concentrated. After purified by silica gel flash chromatography (EtOAc in petroleum ether=40-100%), 4-(2-(2-aminopyridin-3-yl)-5-(difluoromethoxy)-3H-imidazo[4,5-b]pyridin-3-yl)benzyl acetate (130 mg, yield: 43%) was obtained as a brown solid. MS: m/z=426.2 [M+H]⁺.

### Step 5: (4-(2-(2-Aminopyridin-3-yl)-5-(difluoromethoxy)-3H-imidazo[4,5-b]pyridin-3-yl)phenyl)methanol

To a mixture of 4-(2-(2-aminopyridin-3-yl)-5-(difluoromethoxy)-3H-imidazo[4,5-b]pyridin-3-yl)benzyl acetate (130 mg, 306 μmol) in THF (3 mL), MeOH (3 mL), and H₂O (1 mL) was added K₂CO₃ (127 mg, 917 μmol) at 20° C. The reaction mixture was stirred at 20° C. for 0.5 hr. The resulting mixture was quenched with water (10 ml) and extracted with CH₂Cl₂ (10 mL×3). The combined organic layers were washed with brine (10 mL×2), dried over anhydrous Na₂SO₄, filtered and concentrated. (4-(2-(2-Aminopyridin-3-yl)-5-(difluoromethoxy)-3H-imidazo[4,5-b]pyridin-3-yl)phenyl)methanol (117 mg, crude) was obtained as a brown solid, which was used directly into the next step. MS: m/z=384.1 [M+H]⁺.

### Step 6: 3-(3-(4-(Chloromethyl)phenyl)-5-(difluoromethoxy)-3H-imidazo[4,5-b]pyridin-2-yl)pyridin-2-amine

To a solution of (4-(2-(2-aminopyridin-3-yl)-5-(difluoromethoxy)-3H-imidazo[4,5-b]pyridin-3-yl)phenyl)methanol (117 mg, 306 μmol) in CH₂Cl₂ (5 mL) was added SOCl₂ (109 mg, 917 μmol). The reaction mixture was stirred at 20° C. for 2 hr. The reaction mixture was concentrated to give 3-(3-(4-(chloromethyl)phenyl)-5-(difluoromethoxy)-3H-imidazo[4,5-b]pyridin-2-yl)pyridin-2-amine (Intermediate 210, 123 mg, crude) as a gray solid, which was used directly into the next step. MS: m/z=402.1 [M+H]⁺.

### Intermediate 211: 3-(3-(4-(Chloromethyl)phenyl)-5-(4-methoxypyridin-3-yl)-3H-imidazo[4,5-b]pyridin-2-yl)pyridin-2-amine

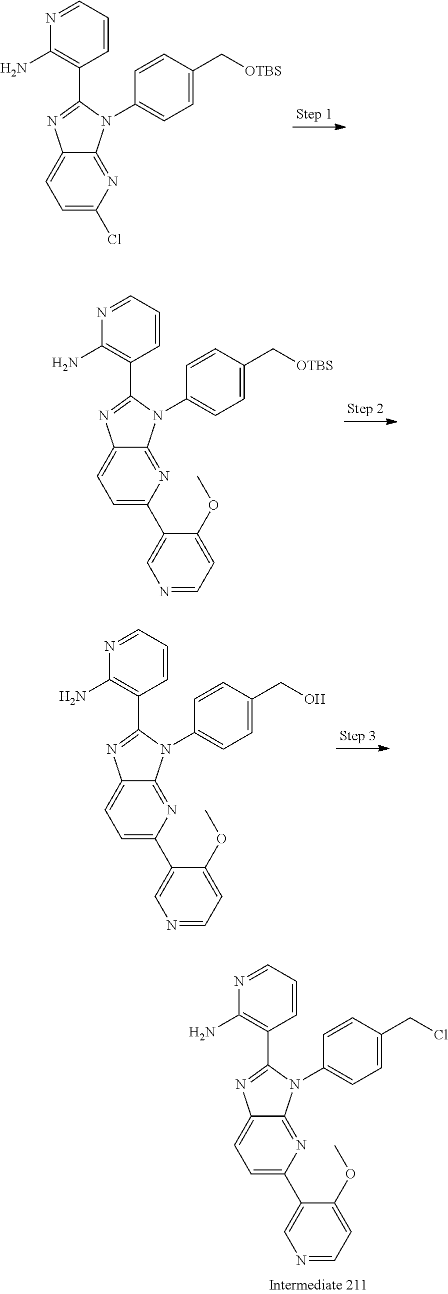

### Step 1: 3-(3-(4-(((Tert-butyldimethylsilyl)oxy)methyl)phenyl)-5-(4-methoxypyridin-3-yl)-3H-imidazo[4,5-b]pyridin-2-yl)pyridin-2-amine

To a mixture of 3-(3-(4-(((tert-butyldimethylsilyl)oxy)methyl)phenyl)-5-chloro-3H-imidazo[4,5-b]pyridin-2-yl)pyridin-2-amine (refer to Intermediate 117 for detail procedures, 400 mg, 858 μmol) and (4-methoxypyridin-3-yl)boronic acid (144 mg, 944 μmol) in 1,4-dioxane (5 mL) and H₂O (1 mL) were added Cs₂CO₃ (839 mg, 2.6 mmol) and Pd(dppf)Cl₂ (62.8 mg, 85.8 μmol), the mixture was stirred at 100° C. for 2 hr under N₂. H₂O (100 mL) was added and extracted with EtOAc (200 mL×2). The combined organic layers were washed with brine (200 mL), dried over anhydrous Na₂SO₄, filtered and concentrated. After purified by silica gel flash chromatography (EtOAc in CH₂Cl₂=0-80%), 3-(3-(4-(((tert-butyldimethylsilyl)oxy)methyl)phenyl)-5-(4-methoxypyridin-3-yl)-3H-imidazo[4,5-b]pyridin-2-yl)pyridin-2-amine (270 mg, yield: 58%) was obtained as a brown solid. MS: m/z=539.2 [M+H]⁺. ¹H NMR (400 MHz, Dimethylsulfoxide-d₆) δ 8.65 (s, 1H) 8.45 (d, J=5.6 Hz, 1H), 8.24 (d, J=8.0 Hz, 1H), 8.00 (dd, J=4.4, 1.6 Hz, 1H), 7.86 (d, J=8.4 Hz, 1H), 7.48-7.44 (m, 4H), 7.22-7.16 (m, 2H), 7.06-7.00 (s, 2H), 6.38 (dd, J=7.6, 4.8 Hz, 1H), 4.80 (s, 2H), 3.92 (s, 3H), 0.90 (s, 9H), 0.09 (s, 6H).

### Step 2: (4-(2-(2-Aminopyridin-3-yl)-5-(4-methoxypyridin-3-yl)-3H-imidazo[4,5-b]pyridin-3-yl)phenyl)methanol

To a mixture of 3-(3-(4-(((tert-butyldimethylsilyl)oxy)methyl)phenyl)-5-(4-methoxypyridin-3-yl)-3H-imidazo[4,5-b]pyridin-2-yl)pyridin-2-amine (260 mg, 482.6 μmol) in THF (3 mL) was added TBAF (1 M in THF, 724 L), the mixture was stirred at 20° C. for 1 hr. The mixture was diluted with H₂O (50 mL) and extracted with EtOAc (100 mL×2). The combined organic layers were washed with brine (100 mL), dried over anhydrous Na₂SO₄, filtered and concentrated, (4-(2-(2-aminopyridin-3-yl)-5-(4-methoxypyridin-3-yl)-3H-imidazo[4,5-b]pyridin-3-yl)phenyl)methanol (200 mg, yield: 98%) was obtained as a yellow solid. MS: m/z=425.1 [M+H]⁺. ¹H NMR (400 MHz, Dimethylsulfoxide-d₆) δ 8.70 (s, 1H), 8.50 (d, J=6.0 Hz, 1H), 8.29 (d, J=8.4 Hz, 1H), 8.05 (dd, J=4.8, 2.0 Hz, 1H), 7.91 (d, J=8.4 Hz, 1H), 7.54-7.46 (m, 4H), 7.30-7.22 (m, 2H), 7.13-6.99 (m, 2H), 6.46 (dd, J=7.6, 4.8 Hz, 1H), 5.38 (t, J=6.0 Hz, 1H), 4.62 (d, J=6.0 Hz, 2H), 3.97 (s, 3H).

### Step 3: 3-(3-(4-(Chloromethyl)phenyl)-5-(4-methoxypyridin-3-yl)-3H-imidazo[4,5-b]pyridin-2-yl)pyridin-2-amine

To a solution of (4-(2-(2-aminopyridin-3-yl)-5-(4-methoxypyridin-3-yl)-3H-imidazo[4,5-b]pyridin-3-yl)phenyl)methanol (200 mg, 471 μmol) in CH₂Cl₂ (3 mL) was added SOCl₂ (168 mg, 1.41 mmol). The mixture was stirred at 25° C. for 2 hr. The mixture was concentrated. 3-(3-(4-(Chloromethyl)phenyl)-5-(4-methoxypyridin-3-yl)-3H-imidazo[4,5-b]pyridin-2-yl)pyridin-2-amine (Intermediate 211, 200 mg, yield: 96%) was obtained as a yellow solid, which was used in the next step directly. MS: m/z=443.1 [M+H]⁺.

### Intermediate 212: 3-(3-(4-(Chloromethyl)phenyl)-5-(2-methoxypyridin-3-yl)-3H-imidazo[4,5-b]pyridin-2-yl)pyridin-2-amine

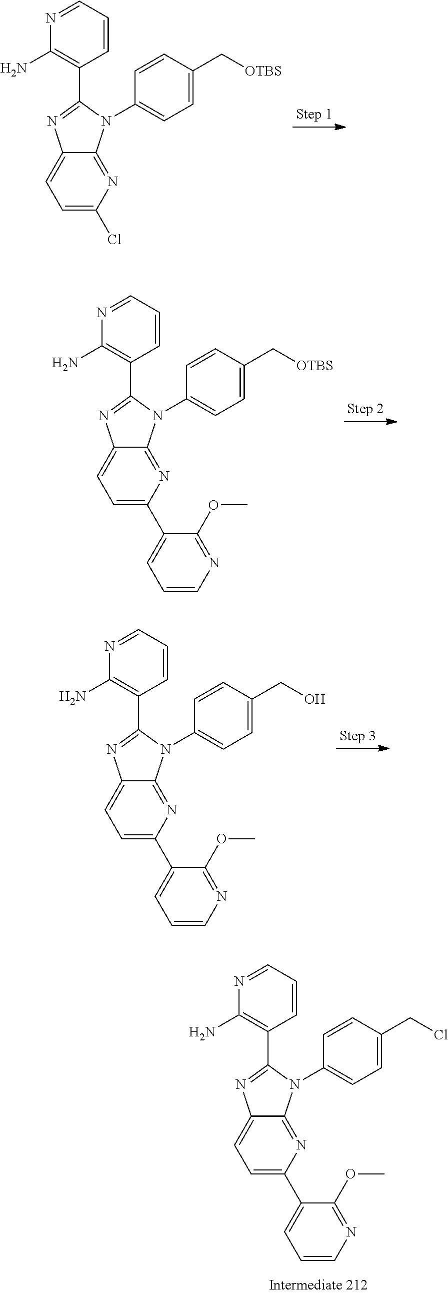

### Step 1: 3-(3-(4-(((Tert-butyldimethylsilyl)oxy)methyl)phenyl)-5-(2-methoxypyridin-3-yl)-3H-imidazo[4,5-b]pyridin-2-yl)pyridin-2-amine

To a solution of 3-(3-(4-(((tert-butyldimethylsilyl)oxy)methyl)phenyl)-5-chloro-3H-imidazo[4,5-b]pyridin-2-yl)pyridin-2-amine (refer to Intermediate 117 for detail procedures, 400 mg, 858 μmol) in 1,4-dioxane (25 mL) and H₂O (5 mL) were added (2-methoxypyridin-3-yl)boronic acid (144 mg, 944 μmol), Pd(dppf)Cl₂ (62.8 mg, 85.8 μmol) and Cs₂CO₃ (839 mg, 2.57 mmol) at 20° C. The reaction mixture was stirred at 100° C. for 2 hr. Water (30 mL) was added, and the resulting mixture was extracted with EtOAc (50 mL×3). The combined organic layers were washed with brine (50 mL×2), dried over anhydrous Na₂SO₄, filtered and concentrated under reduced pressure. 3-(3-(4-(((Tert-butyldimethylsilyl)oxy)methyl)phenyl)-5-(2-methoxypyridin-3-yl)-3H-imidazo[4,5-b]pyridin-2-yl)pyridin-2-amine (460 mg, crude) was obtained as a black brown solid, which was used directly into the next step. MS: m/z=539.2 [M+H]⁺. ¹H NMR (400 MHz, Chloroform-d) δ 8.21-8.16 (m, 2H), 8.12 (s, 2H), 8.00 (d, J=4.4 Hz, 1H), 7.62-7.53 (m, 1H), 7.49 (d, 1=8.4 Hz, 2H), 7.38 (d, 1=8.4 Hz, 2H), 7.20 (d, J=8.0 Hz, 2H), 6.98 (dd, J=7.6, 4.8 Hz, 1H), 6.41 (dd, J=7.6, 4.8 Hz, 1H), 4.85 (s, 2H), 4.05 (s, 3H), 0.97 (s, 9H), 0.14 (s, 6H).

### Step 2: (4-(2-(2-Aminopyridin-3-yl)-5-(2-methoxypyridin-3-yl)-3H-imidazo[4,5-b]pyridin-3-yl)phenyl)methanol

To a solution of 3-(3-(4-(((tert-butyldimethylsilyl)oxy)methyl)phenyl)-5-(2-methoxypyridin-3-yl)-3H-imidazo[4,5-b]pyridin-2-yl)pyridin-2-amine (450 mg, 835 μmol) in THF (30 mL) was added TBAF (1 M in THF, 1.25 mL). The reaction mixture was stirred at 20° C. for 1 hr. Water (30 mL) was added, and the mixture was extracted with EtOAc (50 mL×3). The combined organic layers were washed with brine (50 mL×2), dried over anhydrous Na₂SO₄, filtered and concentrated under reduced pressure. (4-(2-(2-Aminopyridin-3-yl)-5-(2-methoxypyridin-3-yl)-3H-imidazo[4,5-b]pyridin-3-yl)phenyl)methanol (350 mg, crude) was obtained as a black brown solid, which was used directly into the next step. MS: m/z=425.2 [M+H]⁺.

### Step 3: 3-(3-(4-(Chloromethyl)phenyl)-5-(2-methoxypyridin-3-yl)-3H-imidazo[4,5-b]pyridin-2-yl)pyridin-2-amine

To a solution of (4-(2-(2-aminopyridin-3-yl)-5-(2-methoxypyridin-3-yl)-3H-imidazo[4,5-b]pyridin-3-yl)phenyl)methanol (350 mg, 825 μmol) in CH₂Cl₂ (15 mL) was added SOCl₂ (294 mg, 2.47 mmol). The reaction mixture was stirred at 20° C. for 2 hr. The reaction mixture was concentrated. 3-(3-(4-(Chloromethyl)phenyl)-5-(2-methoxypyridin-3-yl)-3H-imidazo[4,5-b]pyridin-2-yl)pyridin-2-amine (Intermediate 212, 360 mg, crude) was obtained as a black brown solid, which was used directly into the next step. MS: m/z=443.1 [M+H]⁺.

### Intermediate 213: 3-(3-(4-(Chloromethyl)phenyl)-5-(1,3-dimethyl-1H-pyrazol-4-yl)-3H-imidazo[4,5-b]pyridin-2-yl)pyridin-2-amine

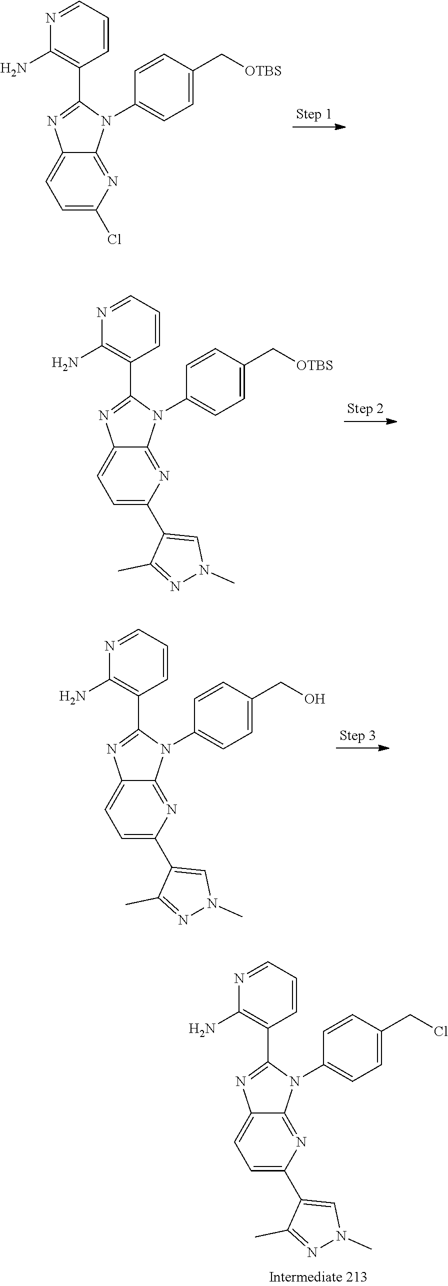 [SMILES: CC(C)(C)[Si](C)(C)OCc1ccc(-n2c(-c3cccnc3N)nc3ccc(Cl)nc32)cc1 ; Cc1nn(C)cc1-c1ccc2nc(-c3cccnc3N)n(-c3ccc(CO[Si](C)(C)C(C)(C)C)cc3)c2n1 ; Cc1nn(C)cc1-c1ccc2nc(-c3cccnc3N)n(-c3ccc(CO)cc3)c2n1 ; Cc1nn(C)cc1-c1ccc2nc(-c3cccnc3N)n(-c3ccc(CCl)cc3)c2n1]

### Step 1: 3-(3-(4-(((Tert-butyldimethylsilyl)oxy)methyl)phenyl)-5-(1,3-dimethyl-1H-pyrazol-4-yl)-3H-imidazo[4,5-b]pyridin-2-yl)pyridin-2-amine

To a solution of 3-(3-(4-(((tert-butyldimethylsilyl)oxy)methyl)phenyl)-5-chloro-3H-imidazo[4,5-b]pyridin-2-yl)pyridin-2-amine (refer to Intermediate 117 for detail procedures, 500 mg, 1.07 mmol) in 1,4-dioxane (30 mL) and H₂O (6 mL) were added (1,3-dimethylpyrazol-4-yl)boronic acid (165 mg, 1.18 mmol), Pd(dppf)Cl₂ (78.5 mg, 107 μmol) and Cs₂CO₃ (1.05 g, 3.22 mmol) at 20° C. The reaction mixture was stirred at 100° C. for 3 hr under N₂. Water (30 mL) was added, and the mixture was extracted with EtOAc (50 mL×3). The combined organic layers were washed with brine (50 mL×2), dried over anhydrous Na₂SO₄, filtered, and concentrated under reduced pressure. 3-(3-(4-(((Tert-butyldimethylsilyl)oxy)methyl)phenyl)-5-(1,3-dimethyl-1H-pyrazol-4-yl)-3H-imidazo[4,5-b]pyridin-2-yl)pyridin-2-amine (560 mg, crude) was obtained as a black brown solid, which was used directly into the next step. MS: m/z=526.3 [M+H]⁺. ¹H NMR (400 MHz, Chloroform-d) δ 8.09-7.99 (m, 2H), 7.72 (s, 1H), 7.48-7.42 (m, 3H), 7.37 (d, J=8.4 Hz, 2H), 7.16-7.09 (m, 1H), 6.69-6.58 (m, 2H), 6.37 (dd, J=7.6, 4.8 Hz, 1H), 4.84 (s, 2H), 3.86 (s, 3H), 2.47 (s, 3H), 0.96 (s, 9H), 0.13 (s, 6H).

### Step 2: (4-(2-(2-Aminopyridin-3-yl)-5-(1,3-dimethyl-1H-pyrazol-4-yl)-3H-imidazo[4,5-b]pyridin-3-yl)phenyl)methanol

To a solution of 3-(3-(4-(((tert-butyldimethylsilyl)oxy)methyl)phenyl)-5-(1,3-dimethyl-1H-pyrazol-4-yl)-3H-imidazo[4,5-b]pyridin-2-yl)pyridin-2-amine (550 mg, 1.05 mmol) in THF (35 mL) was added TBAF (1 M in THF, 1.57 mL). The reaction mixture was stirred at 20° C. for 1 hr. Water (40 mL) was added, and the mixture was extracted with EtOAc (50 mL×3). The combined organic layers were washed with brine (50 mL×2), dried over anhydrous Na₂SO₄, filtered, and concentrated under reduced pressure. (4-(2-(2-Aminopyridin-3-yl)-5-(1,3-dimethyl-1H-pyrazol-4-yl)-3H-imidazo[4,5-b]pyridin-3-yl)phenyl)methanol (430 mg, crude) was obtained as a black brown solid, which was used directly into the next step. MS: m/z=412.2 [M+H]⁺.

### Step 3: 3-(3-(4-(Chloromethyl)phenyl)-5-(1,3-dimethyl-1H-pyrazol-4-yl)-3H-imidazo[4,5-b]pyridin-2-yl)pyridin-2-amine

To a solution of (4-(2-(2-aminopyridin-3-yl)-5-(1,3-dimethyl-1H-pyrazol-4-yl)-3H-imidazo[4,5-b]pyridin-3-yl)phenyl)methanol (430 mg, 1.05 mmol) in CH₂Cl₂ (15 mL) was added SOCl₂ (373 mg, 3.14 mmol). The reaction mixture was stirred at 20° C. for 2 hr. The reaction mixture was concentrated to give 3-(3-(4-(chloromethyl)phenyl)-5-(1,3-dimethyl-1H-pyrazol-4-yl)-3H-imidazo[4,5-b]pyridin-2-yl)pyridin-2-amine (Intermediate 213, 440 mg, crude) as a black brown solid, which was used directly into the next step. ¹H NMR (400 MHz, Methanol-d₄) δ 8.69 (s, 1H), 8.36 (d, J=7.6 Hz, 1H), 8.06 (d, J=4.4 Hz, 1H), 7.98-7.87 (m, 1H), 7.76-7.58 (m, 5H), 6.90 (s, 1H), 4.78 (s, 2H), 4.11 (s, 3H), 2.61 (s, 3H).

### Intermediate 214: 3-(1-(4-(Chloromethyl)phenyl)-1H-imidazo[4,5-c]pyridin-2-yl)pyridin-2-amine

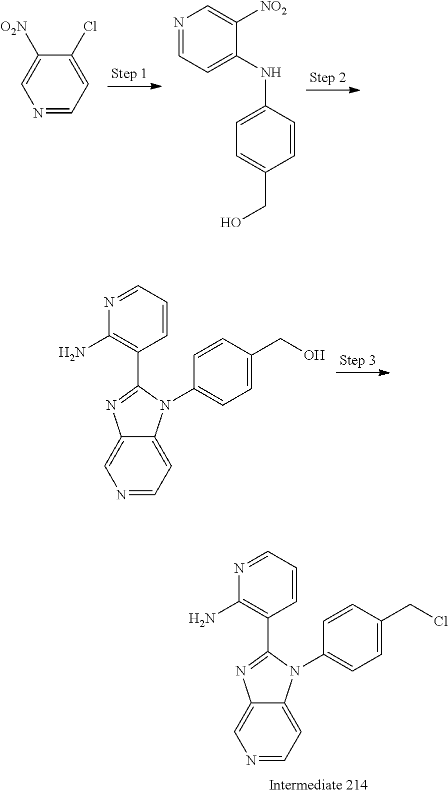 [SMILES: O=[N+]([O-])c1cnccc1Cl ; O=[N+]([O-])c1cnccc1Nc1ccc(CO)cc1 ; Nc1ncccc1-c1nc2cnccc2n1-c1ccc(CO)cc1 ; Nc1ncccc1-c1nc2cnccc2n1-c1ccc(CCl)cc1]

### Step 1. (4-((3-Nitropyridin-4-yl)amino)phenyl)methanol

To a mixture of 4-chloro-3-nitropyridine (2 g, 12.6 mmol) and (4-aminophenyl)methanol (1.71 g, 13.9 mmol) in THF (20 mL) was added DIEA (3.3 g, 25.2 mmol). The mixture was stirred at 80° C. for 12 hr. H₂O (50 mL) was added and the mixture was extracted with EtOAc (50 mL×2). The combined organic layers were washed with brine (50 mL), dried over anhydrous Na₂SO₄, filtered and concentrated. (4-((3-nitropyridin-4-yl)amino)phenyl)methanol (3 g, yield: 97%) was obtained as a yellow solid. MS: m/z=246.1 [M+H]⁺. ¹H NMR (400 MHz, Dimethylsulfoxide-d₆) δ 9.82 (s, 1H), 9.09 (s, 1H), 8.23 (d, J=6.0 Hz, 1H), 7.42 (d, J=8.4 Hz, 2H) 7.31 (d, J=8.0 Hz, 2H), 6.84 (d, J=6.0 Hz, 1H), 5.28 (t, J=6.0 Hz, 1H), 4.53 (d, J=5.4 Hz, 2H).

### Step 2: (4-(2-(2-Aminopyridin-3-yl)-1H-imidazo[4,5-c]pyridin-1-yl)phenyl)methanol

To a mixture of (4-((3-nitropyridin-4-yl)amino)phenyl)methanol (1.5 g, 6.1 mmol) and 2-aminonicotinaldehyde (821.7 mg, 6.7 mmol) in DMSO (10 mL) was added Na₂S₂O₄ (2.7 g, 15.3 mmol). Then it was stirred at 100° C. for 12 hr. The mixture was quenched with H₂O (200 mL) and extracted with CH₂Cl₂ (200 mL×2). The combined organic layers were washed with brine (100 mL), dried over anhydrous Na₂SO₄, filtered and concentrated. After purified by silica gel flash chromatography (EtOAc/EtOH (3/1) in petroleum ether=0-80%), (4-(2-(2-aminopyridin-3-yl)-1H-imidazo[4,5-c]pyridin-1-yl)phenyl)methanol (100 mg, yield: 5%) was obtained as a yellow oil. ¹H NMR (400 MHz, Dimethylsulfoxide-d₆) δ 9.09 (s, 1H), 8.37 (d, J=5.6 Hz, 1H), 8.00 (dd, J=4.8, 1.6 Hz, 1H), 7.50 (d, J=8.0 Hz, 2H), 7.44 (d, J=8.0 Hz, 2H), 7.25-7.19 (m, 2H), 6.90 (s, 2H), 6.41 (dd, J=7.6, 4.8 Hz, 1H), 5.38 (t, J=5.6 Hz, 1H), 4.59 (d, J=5.6 Hz, 2H).

### Step 3: 3-(1-(4-(Chloromethyl)phenyl)-1H-imidazo[4,5-c]pyridin-2-yl)pyridin-2-amine

To a solution of (4-(2-(2-aminopyridin-3-yl)-1H-imidazo[4,5-c]pyridin-1-yl)phenyl)methanol (100 mg, 315 μmol) in CH₂Cl₂ (5 mL) was added SOCl₂ (112 mg, 945 μmol) at 25° C. The mixture was stirred at 25° C. for 2 hr. The reaction mixture was concentrated to give 3-(1-(4-(chloromethyl)phenyl)-1H-imidazo[4,5-c]pyridin-2-yl)pyridin-2-amine (Intermediate 214, 102 mg, yield. 96%) as a yellow solid, which was used directly without purification. MS: m/z=335.9 [M+H]⁺.

### Intermediate 215: 3-(3-(4-(Chloromethyl)phenyl)-5-(3-methoxypyridin-2-yl)-3H-imidazo[4,5-b]pyridin-2-yl)pyridin-2-amine

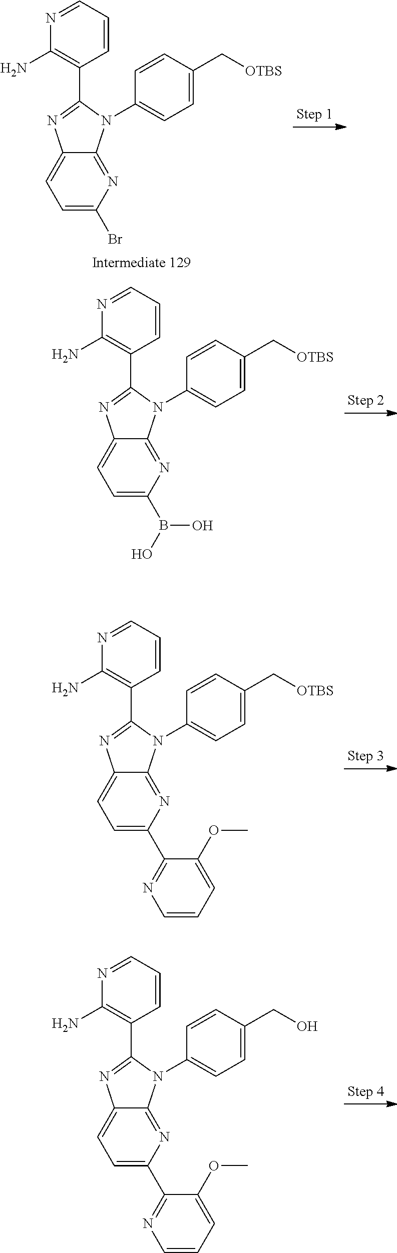 [SMILES: CC(C)(C)[Si](C)(C)OCc1ccc(-n2c(-c3cccnc3N)nc3ccc(Br)nc32)cc1 ; CC(C)(C)[Si](C)(C)OCc1ccc(-n2c(-c3cccnc3N)nc3ccc(B(O)O)nc32)cc1 ; COc1cccnc1-c1ccc2nc(-c3cccnc3N)n(-c3ccc(CO[Si](C)(C)C(C)(C)C)cc3)c2n1 ; COc1cccnc1-c1ccc2nc(-c3cccnc3N)n(-c3ccc(CO)cc3)c2n1]

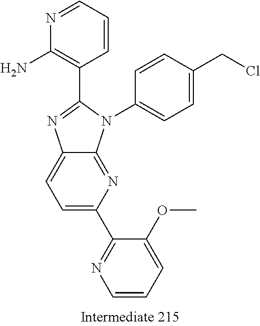 [SMILES: COc1cccnc1-c1ccc2nc(-c3cccnc3N)n(-c3ccc(CCl)cc3)c2n1]

### Step 1: (2-(2-Aminopyridin-3-yl)-3-(4-(((tert-butyldimethylsilyl)oxy)methyl)phenyl)-3H-imidazo[4,5-b]pyridin-5-yl)boronic acid

A mixture of Intermediate 129 (500 mg, 979 μmol), 4,4,4′,4′,5,5,5′,5′-octamethyl-2,2′-bi(1,3,2-dioxaborolane) (497 mg, 1.96 mmol), KOAc (288 mg, 2.94 mmol) and Pd(dppf)Cl₂ (71.7 mg, 97.9 μmol) in 1,4-dioxane (2 mL) was degassed and purged with N₂ three times. The mixture was stirred at 80° C. for 16 hr under N₂ atmosphere. (2-(2-Aminopyridin-3-yl)-3-(4-(((tert-butyldimethylsilyl)oxy)methyl)phenyl)-3H-imidazo[4,5-b]pyridin-5-yl)boronic acid was obtained as a black liquid, which was used in the next step without work-up and purification. MS: m/z=476.2 [M+H]⁺.

### Step 2: 3-(3-(4-(((tert-Butyldimethylsilyl)oxy)methyl)phenyl)-5-(3-methoxypyridin-2-yl)-3H-imidazo[4,5-b]pyridin-2-yl)pyridin-2-amine

A mixture of (2-(2-aminopyridin-3-yl)-3-(4-(((tert-butyldimethylsilyl)oxy)methyl)phenyl)-3H-imidazo[4,5-b]pyridin-5-yl)boronic acid (470 mg, 989 μmol), 2-bromo-3-methoxypyridine (186 mg, 989 μmol), Pd(dppf)Cl₂ (72.3 mg, 98.9 μmol) and Cs₂CO₃ (966 mg, 2.97 mmol) in 1,4-dioxane (5 mL) and H₂O (1 mL) was degassed and purged with N₂ three times. The mixture was stirred at 90° C. for 16 hr under N₂ atmosphere. The reaction mixture was quenched with H₂O (5 mL), diluted with CH₂C12 (10 mL), and extracted with CH₂C12 (15 mL×2). The combined organic layers were washed with brine (30 mL), dried over Na₂SO₄, filtered, and concentrated under reduced pressure. After purified by silica gel flash chromatography (Eluent of 30%˜61% EtOAc in petroleum ether), 3-(3-(4-((tert-butyldimethylsilyl)oxy)methyl)phenyl)-5-(3-methoxypyridin-2-yl)-3H-imidazo[4,5-b]pyridin-2-yl)pyridin-2-amine (280 mg, yield: 47% for two steps) was obtained as a black solid. MS: m/z=539.2 [M+H]⁺. ¹H NMR (400 MHz, Chloroform-d) δ 8.32 (dd, J=4.4, 1.6 Hz, 1H), 8.15 (d, J=8.4 Hz, 1H), 8.04 (dd, J=4.8, 1.6 Hz, 1H), 7.84 (d, J=8.4 Hz, 1H), 7.46-7.40 (m, 4H), 7.34-7.27 (m, 2H), 7.10 (dd, J=7.6, 1.6 Hz, 1H), 6.69 (br s, 2H), 6.36 (dd, J=8.0, 4.8 Hz, 1H), 4.80 (s, 2H), 3.83 (s, 3H), 0.95 (s, 9H), 0.12 (s, 6H).

### Step 3: (4-(2-(2-Aminopyridin-3-yl)-5-(3-methoxypyridin-2-yl)-31-imidazo[4,5-b]pyridin-3-yl)phenyl)methanol

To a solution of 3-(3-(4-(((tert-butyldimethylsilyl)oxy)methyl)phenyl)-5-(3-methoxypyridin-2-yl)-3H-imidazo[4,5-b]pyridin-2-yl)pyridin-2-amine (250 mg, 464 μmol) in THF (5 mL) was added TBAF (1.4 mL, 1.4 mmol, 1 M in THF). The mixture was stirred at 25° C. for 0.5 hr. The reaction mixture was quenched with H₂O (5 mL) at 25° C., diluted with CH₂Cl₂ (10 mL), and extracted with CH₂Cl₂ (15 mL×3). The combined organic layers were washed with brine (50 mL×2), dried over Na₂SO₄, filtered and concentrated under reduced pressure. After triturated with EtOAc: petroleum ether=1:10 (10 mL) at 25° C. for 10 min, (4-(2-(2-aminopyridin-3-yl)-5-(3-methoxypyridin-2-yl)-3H-imidazo[4,5-b]pyridin-3-yl)phenyl)methanol (175 mg, yield: 84%) was obtained as a brown solid. MS: m/z=425.2 [M+H]˜. ¹H NMR (400 MHz, Dimethylsulfoxide-d₆) δ 8.26-8.20 (m, 2H), 7.99 (dd, J=4.8, 2.0 Hz, 1H), 7.67 (d, 1=8.0 Hz, 1H), 7.61-7.53 (m, 1H), 7.46-7.38 (m, 5H), 7.23 (dd, J=7.6, 1.6 Hz, 1H), 6.96 (br s, 2H), 6.42 (dd, J=7.6, 5.2 Hz, 1H), 5.34 (t, J=6.0 Hz, 1H), 4.56 (d, J=6.0 Hz, 2H), 3.77 (s, 3H).

### Step 4: 3-(3-(4-(Chloromethyl)phenyl)-5-(3-methoxypyridin-2-yl)-3H-imidazo[4,5-b]pyridin-2-yl)pyridin-2-amine

To a solution of (4-(2-(2-aminopyridin-3-yl)-5-(3-methoxypyridin-2-yl)-3H-imidazo[4,5-b]pyridin-3-yl)phenyl)methanol (175 mg, 412 μmol) in CH₂Cl₂ (5 mL) was added SOCl₂ (245 mg, 2.06 mmol). The mixture was stirred at 25° C. for 0.5 hr. The reaction was concentrated under reduced pressure to give 3-(3-(4-(chloromethyl)phenyl)-5-(3-methoxypyridin-2-yl)-3H-imidazo[4,5-b]pyridin-2-yl)pyridin-2-amine (Intermediate 215, 198 mg, HCl salt) as a brown solid. MS: m/z=443.1 [M+H]⁺. ¹H NMR (400 MHz, Methanol-d₄) δ8.62 (d, J=8.4 Hz, 1H), 8.54-8.45 (m, 3H), 8.09-8.04 (m, 2H), 7.86 (d, J=7.2 Hz, 1H), 7.74-7.69 (m, 2H), 7.66-7.62 (m, 2H), 6.91-6.83 (m, 1H), 4.79 (s, 2H), 4.26 (s, 3H).

### Intermediate 216: 3-(3-(4-(Chloromethyl)phenyl)-5-(1-methyl-1H-1,2,3-triazol-5-yl)-3H-imidazo[4,5-b]pyridin-2-yl)pyridin-2-amine

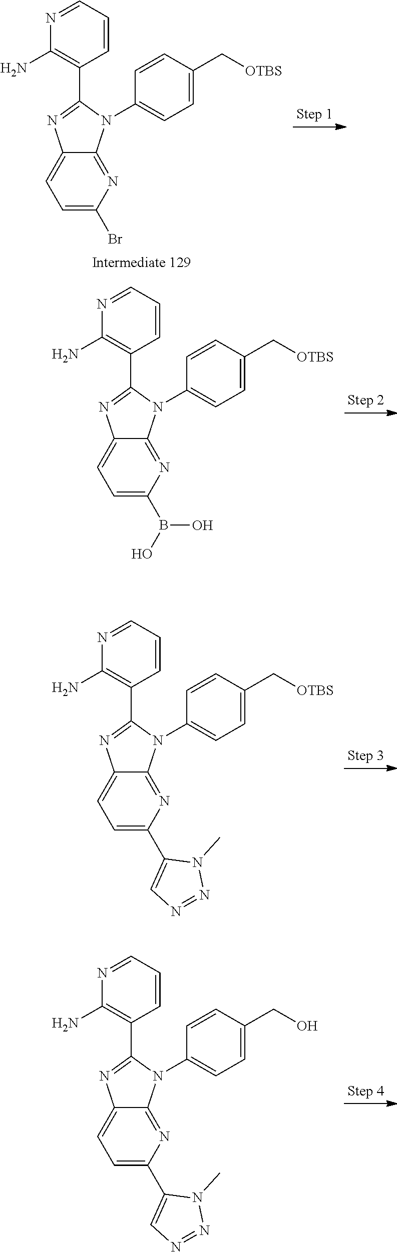 [SMILES: CC(C)(C)[Si](C)(C)OCc1ccc(-n2c(-c3cccnc3N)nc3ccc(Br)nc32)cc1 ; CC(C)(C)[Si](C)(C)OCc1ccc(-n2c(-c3cccnc3N)nc3ccc(B(O)O)nc32)cc1 ; Cn1nncc1-c1ccc2nc(-c3cccnc3N)n(-c3ccc(CO[Si](C)(C)C(C)(C)C)cc3)c2n1 ; Cn1nncc1-c1ccc2nc(-c3cccnc3N)n(-c3ccc(CO)cc3)c2n1]

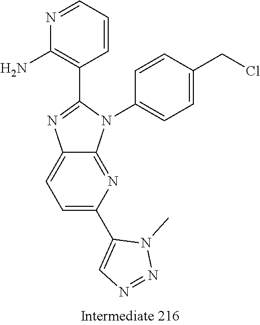 [SMILES: Cn1nncc1-c1ccc2nc(-c3cccnc3N)n(-c3ccc(CCl)cc3)c2n1]

### Step 1: (2-(2-Aminopyridin-3-yl)-3-(4-(((tert-butyldimethylsilyl)oxy)methyl)phenyl)-3H-imidazo[4,5-b]pyridin-5-yl)boronic acid

A mixture of Intermediate 129 (1 g, 1.96 mmol), 4,4,4′,4′,5,5,5′,5′-octamethyl-2,2′-bi(1,3,2-dioxaborolane) (995 mg, 3.92 mmol), KOAc (577 mg, 5.88 mmol) and Pd(dppf)Cl₂ (143 mg, 196 mmol) in 1,4-dioxane (5 mL) was degassed and purged with N₂ three times. The mixture was stirred at 90° C. for 16 hr under N₂ atmosphere. (2-(2-Aminopyridin-3-yl)-3-(4-(((tert-butyldimethylsilyl)oxy)methyl)phenyl)-3H-imidazo[4,5-b]pyridin-5-yl)boronic acid was obtained as a black liquid and used in the next step without work-up and purification. MS: m/z=476.2 [M+H]⁺.

### Step 2: 3-(3-(4-(((tert-Butyldimethylsilyl)oxy)methyl)phenyl)-5-(1-methyl-1H-1,2,3-triazol-5-yl)-3H-imidazo[4,5-b]pyridin-2-yl)pyridin-2-amine

A mixture of (2-(2-aminopyridin-3-yl)-3-(4-(((tert-butyldimethylsilyl)oxy)methyl)phenyl)-3H-imidazo[4,5-b]pyridin-5-yl)boronic acid (400 mg, 841 μmol), 5-bromo-1-methyl-1H-1,2,3-triazole (164 mg, 1.01 mmol), Pd(dppf)Cl₂ (61.6 mg, 84.1 μmol) and Cs₂CO₃ (822 mg, 2.52 mmol) in 1,4-dioxane (4 mL) and H₂O (1 mL) was degassed and purged with N₂ three times. The mixture was stirred at 90° C. for 16 hr under N₂ atmosphere. The reaction mixture was poured into H₂O (15 mL) and extracted with EtOAc (15 mL×3). The combined organic layers were dried over anhydrous Na₂SO₄, filtered, and concentrated under reduced pressure. After purified by silica gel flash chromatography (Eluent of 0%˜50% EtOAc in petroleum ether), 3-(3-(4-(((tert-butyldimethylsilyl)oxy)methyl)phenyl)-5-(1-methyl-1H-1,2,3-triazol-5-yl)-3H-imidazo[4,5-b]pyridin-2-yl)pyridin-2-amine (210 mg, yield: 49% for two steps) was obtained as a brown solid. MS: m/z=513.2 [M+H]⁺. ¹HNMR (400 MHz, Dimethysulfoxide-d₆) δ 8.36-8.33 (m, 2H), 8.15 (dd, J=5.2, 2.0 Hz, 1H), 7.91 (d, J=8.4 Hz, 1H), 7.53-7.46 (m, 4H), 7.25 (dd, J=7.6, 1.6 Hz, 1H), 7.02 (s, 2H), 6.41 (dd, J=7.6, 4.8 Hz, 1H), 4.80 (s, 2H), 4.16 (s, 3H), 0.91 (s, 9H), 0.09 (s, 6H).

### Step 3: (4-(2-(2-Aminopyridin-3-yl)-5-(1-methyl-1H-1,2,3-triazol-5-yl)-3H-imidazo[4,5-b]pyridin-3-yl)phenyl)methanol

To a solution of 3-(3-(4-(((tert-butyldimethylsilyl)oxy)methyl)phenyl)-5-(1-methyl-1H-1,2,3-triazol-5-yl)-3H-imidazo[4,5-b]pyridin-2-yl)pyridin-2-amine (210 mg, 410 μmol) in THF (1 mL) was added TBAF (1 mL, 1 M in THF). The mixture was stirred at 25° C. for 1 hr. The reaction mixture was poured into H₂O (15 mL) and extracted with EtOAc (15 mL×3). The combined organic layers were dried over anhydrous Na₂SO₄, filtered, and concentrated under reduced pressure. (4-(2-(2-Aminopyridin-3-yl)-5-(1-methyl-1H-1,2,3-triazol-5-yl)-3H-imidazo[4,5-b]pyridin-3-yl)phenyl)methanol (150 mg, crude) was obtained as a brown solid. MS: m/z=399.1 [M+H]⁺.

### Step 4: 3-(3-(4-(Chloromethyl)phenyl)-5-(1-methyl-1H-1,2,3-triazol-5-yl)-3H-imidazo[4,5-b]pyridin-2-yl)pyridin-2-amine

To a solution of (4-(2-(2-aminopyridin-3-yl)-5-(1-methyl-1H-1,2,3-triazol-5-yl)-3H-imidazo[4,5-b]pyridin-3-yl)phenyl)methanol (150 mg, 376 μmol) in CH₂Cl₂ (2 mL) was added SOCl₂ (1.09 g, 9.16 mmol). The mixture was stirred at 25° C. for 1 hr. The reaction was concentrated under reduced pressure to give 3-(3-(4-(chloromethyl)phenyl)-5-(1-methyl-1H-1,2,3-triazol-5-yl)-3H-imidazo[4,5-b]pyridin-2-yl)pyridin-2-amine (Intermediate 216, 155 mg, HCl salt) as a brown solid. MS: m/z=417.1 [M+H]⁺.

### Intermediate 217: 2-(2-Aminopyridin-3-yl)-3-(4-(chloromethyl)phenyl)-6-methyl-3H-imidazo[4,5-b]pyridine-5-carbonitrile

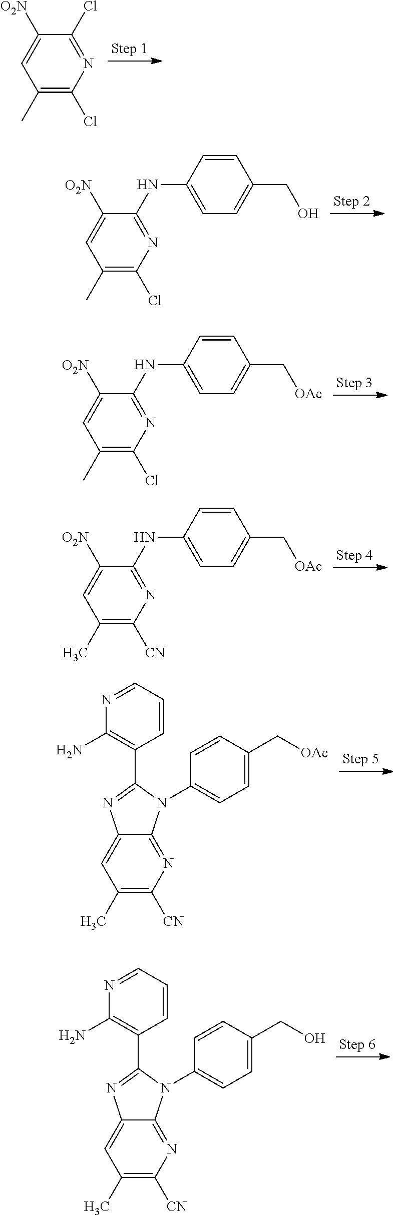

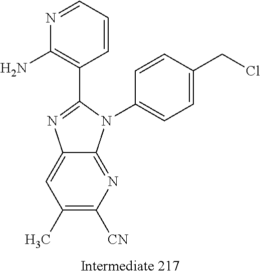 [SMILES: Cc1cc2nc(-c3cccnc3N)n(-c3ccc(CCl)cc3)c2nc1C#N]

### Step 1: (4-((6-Chloro-5-methyl-3-nitropyridin-2-yl)amino)phenyl)methanol

To a solution of 2,6-dichloro-3-methyl-5-nitropyridine (10 g, 48.3 mmol) and (4-aminophenyl)methanol (5.35 g, 43.5 mmol) in 1,4-dioxane (100 mL) was added DLEA (12.5 g, 96.6 mmol). The mixture was stirred at 60° C. for 16 hr. The reaction mixture was diluted with H₂O (100 mL) and extracted with CH₂Cl₂ (100 mL×3). The combined organic layers were washed with brine (100 mL×4), dried over Na₂SO₄, filtered and concentrated under reduced pressure. (4-((6-Chloro-5-methyl-3-nitropyridin-2-yl)amino)phenyl)methanol (14 g, crude) was obtained as a red solid. MS: m/z=294.0 [M+H]⁺.

### Step 2: 4-((6-Chloro-5-methyl-3-nitropyridin-2-yl)amino)benzyl acetate

To a solution of (4-((6-chloro-5-methyl-3-nitropyridin-2-yl)amino)phenyl)methanol (14 g, 47.7 mmol) and TEA (14.5 g, 143 mmol) in CH₂Cl₂ (200 mL) were added Ac₂O (7.3 g, 71.5 mmol) and DMAP (582 mg, 4.77 mmol). The mixture was stirred at 25° C. for 1 hr. The reaction mixture was diluted with H₂O (200 mL) and extracted with CH₂Cl₂ (200 mL×3). The combined organic layers were washed with brine (300 mL), dried over Na₂SO₄, filtered and concentrated under reduced pressure. After purified by silica gel flash chromatography (Eluent of 0˜10% EtOAc in petroleum ether), 4-((6-chloro-5-methyl-3-nitropyridin-2-yl)amino)benzyl acetate (7 g, yield: 44% for two steps) was obtained as a red solid. MS: m/z=336.0 [M+H]+. ¹H NMR (400 MHz, Dimethylsulfoxide-d₆) δ 9.98 (s, 1H), 8.56 (s, 1H), 7.60 (d, J=8.4 Hz, 2H), 7.38 (d, J=8.8 Hz, 2H), 5.06 (s, 2H), 2.29 (s, 3H), 2.07 (s, 3H).

### Step 3: 4-((6-Cyano-5-methyl-3-nitropyridin-2-yl)amino)benzyl acetate

To a solution of 4-((6-chloro-5-methyl-3-nitropyridin-2-yl)amino)benzyl acetate (2.4 g, 7.15 mmol) in DMF (30 mL) was added CuCN (3.2 g, 35.7 mmol). The mixture was stirred at 180° C. for 8 hr. The reaction mixture was quenched with H₂O (50 mL) at 25° C. and extracted with CH₂Cl₂ (100 mL×3). The combined organic layers were washed with brine (100 mL×5), dried over Na₂SO₄, filtered and concentrated under reduced pressure. After purified by silica gel flash chromatography (Eluent of 0˜5% EtOAc in petroleum ether), 4-((6-cyano-5-methyl-3-nitropyridin-2-yl)amino)benzyl acetate (150 mg, yield: 6.4%) was obtained as a red solid. MS: m/z=326.9 [M+H]⁺. ¹H NMR (400 MHz, Dimethylsulfoxide-d₆) δ 9.80 (s, 1H), 8.66 (s, 1H), 7.57 (d, J=8.4 Hz, 2H), 7.38 (d, J=8.0 Hz, 2H), 5.06 (s, 2H), 2.44 (s, 3H), 2.07 (s, 3H).

### Step 4: 4-(2-(2-Aminopyridin-3-yl)-5-cyano-6-methyl-3H-imidazo[4,5-b]pyridin-3-yl)benzyl acetate

A mixture of 4-((6-cyano-5-methyl-3-nitropyridin-2-yl)amino)benzyl acetate (140 mg, 429 μmol) and 2-aminonicotinaldehyde (63 mg, 515 μmol) in DMSO (2 mL) was added Na₂S₂O₄ (352 mg, 1.72 mmol) at 25° C. The mixture was stirred at 80° C. for 16 hr. The reaction mixture was quenched with H₂O (10 mL) at 25° C., diluted with CH₂Cl₂ (10 mL), and extracted with CH₂Cl₂ (10 mL×3). The combined organic layers were washed with brine (10 mL×3), dried over Na₂SO₄, filtered, and concentrated under reduced pressure. After purified by prep-TLC (100% EtOAc), 4-(2-(2-aminopyridin-3-yl)-5-cyano-6-methyl-3H-imidazo[4,5-b]pyridin-3-yl)benzyl acetate (30 mg, yield: 18%) was obtained as a yellow solid. MS: m/z=399.4 [M+H]+.

### Step 5: 2-(2-Aminopyridin-3-yl)-3-(4-(hydroxymethyl)phenyl)-6-methyl-3H-imidazo[4,5-b]pyridine-5-carbonitrile

To a solution of 4-(2-(2-aminopyridin-3-yl)-5-cyano-6-methyl-3H-imidazo[4,5-b]pyridin-3-yl)benzyl acetate (30 mg, 75.3 μmol) in THF (1 mL) were added K₂CO₃ (31.2 mg, 226 mmol), MeOH (1 mL) and H₂O (1 mL). The mixture was stirred at 25° C. for 0.5 hr. The reaction mixture was diluted with H₂O (20 mL) and extracted with CH₂Cl₂ (20 mL×3). The combined organic layers were washed with brine (40 mL), dried over Na₂SO₄, filtered and concentrated under reduced pressure to give 2-(2-aminopyridin-3-yl)-3-(4-(hydroxymethyl)phenyl)-6-methyl-3H-imidazo[4,5-b]pyridine-5-carbonitrile (27 mg, crude) as a yellow solid. MS: m/z=357.2 [M+H]⁺.

### Step 6: 2-(2-Aminopyridin-3-yl)-3-(4-(chloromethyl)phenyl)-6-methyl-3H-imidazo[4,5-b]pyridine-5-carbonitrile

To a solution of 2-(2-aminopyridin-3-yl)-3-(4-(hydroxymethyl)phenyl)-6-methyl-3H-imidazo[4,5-b]pyridine-5-carbonitrile (27 mg, 75.8 μmol) in CH₂Cl₂ (2 mL) was added SOCl₂ (0.3 mL). The mixture was stirred at 40° C. for 1 hr. The mixture was concentrated under reduced pressure to give 2-(2-aminopyridin-3-yl)-3-(4-(chloromethyl)phenyl)-6-methyl-3H-imidazo[4,5-b]pyridine-5-carbonitrile (Intermediate 217, 28 mg, crude) was obtained as a yellow solid. MS: m/z=375.0 [M+H]⁺.

### Intermediate 218: 3-(3-(4-(Chloromethyl)phenyl)-5-(5-methyl-1,3,4-oxadiazol-2-yl)-3H-imidazo[4,5-b]pyridin-2-yl)pyridin-2-amine

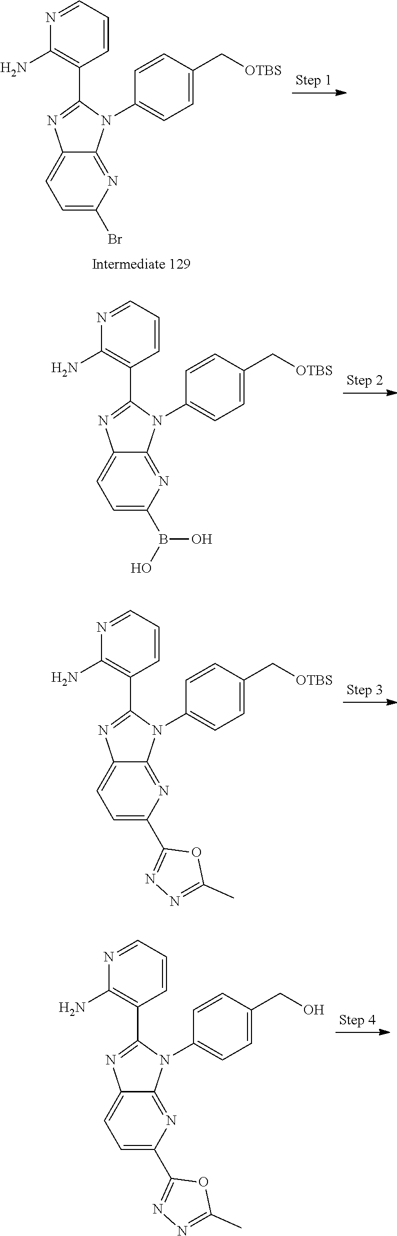

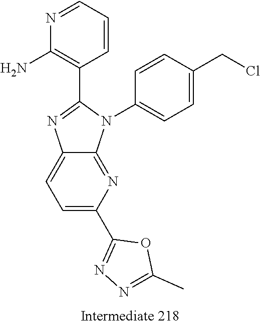 [SMILES: Cc1nnc(-c2ccc3nc(-c4cccnc4N)n(-c4ccc(CCl)cc4)c3n2)o1]

### Step 1: (2-(2-Aminopyridin-3-yl)-3-(4-(((tert-butyldimethylsilyl)oxy)methyl)phenyl)-3H-imidazo[4,5-b]pyridin-5-yl)boronic acid

A mixture of Intermediate 129 (1.5 g, 2.94 mmol), 4,4,5,5-tetramethyl-2-(4,4,5,5-tetramethyl-1,3,2-dioxaborolan-2-yl)-1,3,2-dioxaborolane (1.49 g, 5.88 mmol), KOAc (865 mg, 8.81 mmol) and Pd(dppf)Cl₂ (215 mg, 294 μmol) in 1,4-dioxane (5 mL) was degassed and purged with N₂ three times, and then the mixture was stirred at 90° C. for 16 hr under N₂ atmosphere. (2-(2-Aminopyridin-3-yl)-3-(4-(((tert-butyldimethylsilyl)oxy)methyl)phenyl)-3H-imidazo[4,5-b]pyridin-5-yl)boronic acid (1.5 g, crude) was obtained as a black brown solid, which was directly used in the next step without further purification. MS: m/z=476.2 [M+H]⁺.

### Step 2: 3-(3-(4-(((tert-Butyldimethylsilyl)oxy)methyl)phenyl)-5-(5-methyl-1,3,4-oxadiazol-2-yl)-3H-imidazo[4,5-b]pyridin-2-yl)pyridin-2-amine

A mixture of (2-(2-aminopyridin-3-yl)-3-(4-(((tert-butyldimethylsilyl)oxy)methyl)phenyl)-3H-imidazo[4,5-b]pyridin-5-yl)boronic acid (475 mg, 999 μmol), 2-bromo-5-methyl-1,3,4-oxadiazole (163 mg, 999 μmol), Cs₂CO₃ (977 mg, 3.00 mmol) and Pd(dppf)Cl₂ (73 mg, 100 μmol) in 1,4-dioxane (4 mL) and H₂O (1 mL) was degassed and purged with N₂ three times. The mixture was stirred at 90° C. for 16 hr under N₂ atmosphere. The reaction mixture was diluted with H₂O (30 mL) and extracted with EtOAc (20 mL×3). The combined organic layers were washed with brine (30 mL), dried over Na₂SO₄, filtered and concentrated under reduced pressure. After purified by silica gel flash chromatography (Eluent of 0˜100% EtOAc in petroleum ether), 3-(3-(4-(((tert-butyldimethylsilyl)oxy)methyl)phenyl)-5-(5-methyl-1,3,4-oxadiazol-2-yl)-3H-imidazo[4,5-b]pyridin-2-yl)pyridin-2-amine (150 mg, yield: 23% for two steps) was obtained as a brown solid. MS: m/z=514.1 [M+H]⁺. ¹H NMR (400 MHz, Dimethysulfoxide-d₆) 8.40 (d, J=8.4 Hz, 1H), 8.18 (d, J=8.4 Hz, 1H), 8.01 (dd, J=4.8, 2.0 Hz, 1H), 7.53-7.44 (m, 4H), 7.25-7.18 (m, 1H), 7.04-6.92 (m, 2H), 6.39 (dd, J=7.6, 4.8 Hz, 1H), 4.82 (s, 2H), 2.56 (s, 3H), 0.93 (s, 9H), 0.12 (s, 6H).

### Step 3: (4-(2-(2-Aminopyridin-3-yl)-5-(5-methyl-1,3,4-oxadiazol-2-yl)-3H-imidazo[4,5-b]pyridin-3-yl)phenyl)methanol

To a solution of 3-(3-(4-(((tert-butyldimethylsilyl)oxy)methyl)phenyl)-5-(5-methyl-1,3,4-oxadiazol-2-yl)-3H-imidazo[4,5-b]pyridin-2-yl)pyridin-2-amine (150 mg, 292 μmol) in THF (3 mL) was added TBAF (0.6 mL, 1 M in THF). The mixture was stirred at 25° C. for 0.5 hr. The reaction mixture was diluted with H₂O (30 mL) and extracted with EtOAc (20 mL×3). The combined organic layers were washed with brine (30 mL), dried over Na₂SO₄, filtered, and concentrated under reduced pressure. The crude product was used in the next step without further purification. (4-42-(2-Aminopyridin-3-yl)-5-(5-methyl-1,3,4-oxadiazol-2-yl)-3H-imidazo[4,5-b]pyridin-3-yl)phenyl)methanol (100 mg, crude) was obtained as a yellow solid. MS: m/z=400.1 [M+H]⁺.

### Step 4: 3-(3-(4-(Chloromethyl)phenyl)-5-(5-methyl-1,3,4-oxadiazol-2-yl)-3H-imidazo[4,5-b]pyridin-2-yl)pyridin-2-amine

To a solution of (4-(2-(2-aminopyridin-3-yl)-5-(5-methyl-1,3,4-oxadiazol-2-yl)-3H-imidazo[4,5-b]pyridin-3-yl)phenyl)methanol (100 mg, 250 μmol) in CH₂Cl₂ (2 mL) was added SOCl₂ (60 mg, 501 μmol). The mixture was stirred at 40° C. for 0.5 hr. The mixture was concentrated under reduced pressure. The crude product was used in the next step without further purification. 3-(3-(4-(Chloromethyl)phenyl)-5-(5-methyl-1,3,4-oxadiazol-2-yl)-3H-imidazo[4,5-b]pyridin-2-yl)pyridin-2-amine (Intermediate 218, 100 mg, HCl salt) was obtained as a yellow solid. MS: m/z=418.0 [M+H]⁺.

### Intermediate 219: 3-(3-(4-(chloromethyl)phenyl)-5-(1-methyl-1H-1,2,4-triazol-5-yl)-3H-imidazo[4,5-b]pyridin-2-yl)pyridin-2-amine

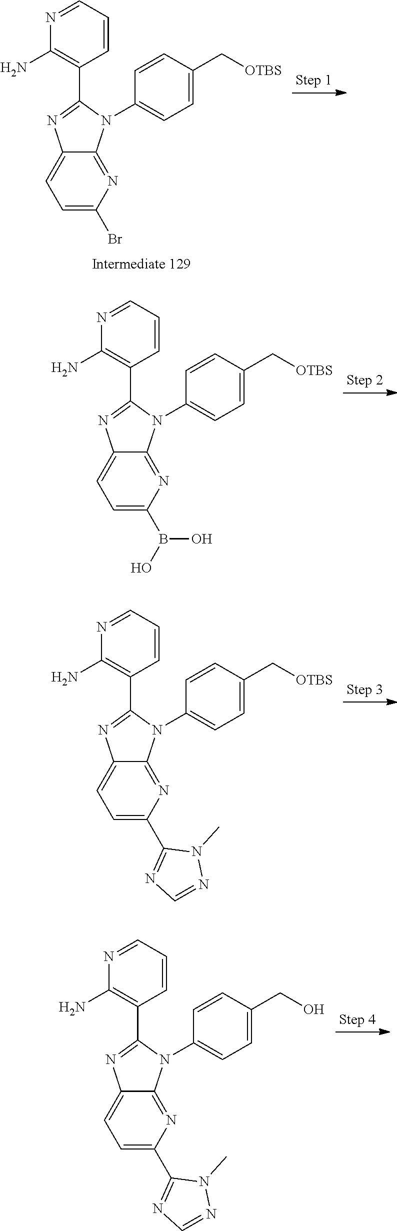

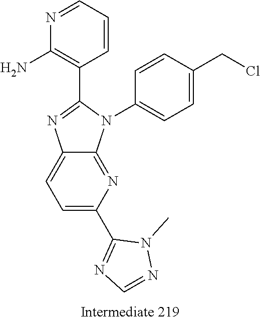 [SMILES: Cn1ncnc1-c1ccc2nc(-c3cccnc3N)n(-c3ccc(CCl)cc3)c2n1]

### Step 1: (2-(2-Aminopyridin-3-yl)-3-(4-(((tert-butyldimethylsilyl)oxy)methyl)phenyl)-3H-imidazo[4,5-b]pyridin-5-yl)boronic acid

A mixture of Intermediate 129 (1 g, 1.96 mmol), 4,4,4′,4′,5,5,5′,5′-octamethyl-2,2′-bi(1,3,2-dioxaborolane) (995 mg, 3.92 mmol), KOAc (577 mg, 5.88 mmol) and Pd(dppf)Cl₂ (143 mg, 196 mmol) in 1,4-dioxane (5 mL) was degassed and purged with N₂ three times. The mixture was stirred at 90° C. for 16 hr under N₂ atmosphere. (2-(2-Aminopyridin-3-yl)-3-(4-(((tert-butyldimethylsilyl)oxy)methyl)phenyl)-3H-imidazo[4,5-b]pyridin-5-yl)boronic acid was obtained as a black liquid, which was used in the next step without work-up and purification. MS: m/z=476.1 [M+H]⁺.

### Step 2: 3-(3-(4-(((Tert-butyldimethylsilyl)oxy)methyl)phenyl)-5-(1-methyl-1H-1,2,4-triazol-5-yl)-3H-imidazo[4,5-b]pyridin-2-yl)pyridin-2-amine

A mixture of (2-(2-aminopyridin-3-yl)-3-(4-(((tert-butyldimethylsilyl)oxy)methyl)phenyl)-3H-imidazo[4,5-b]pyridin-5-yl)boronic acid (470 mg, 988 μmol), 5-bromo-1-methyl-1H-1,2,4-triazole (192 mg, 1.19 mmol), Pd(dppf)Cl₂ (72.3 mg, 98.9 μmol) and Cs₂CO₃ (966 mg, 2.97 mmol) in 1,4-dioxane (4 mL) and H₂O (1 mL) was degassed and purged with N₂ three times. The mixture was stirred at 90° C. for 16 hr under N₂ atmosphere. The reaction mixture was poured into H₂O (15 mL) and extracted with EtOAc (15 mL×3). The organic phase was dried over anhydrous Na₂SO₄, filtered, and concentrated under reduced pressure. After purified by silica gel flash chromatography (Eluent of 0%˜50% EtOAc in petroleum ether), 3-(3-(4-(((tert-butyldimethylsilyl)oxy)methyl)phenyl)-5-(1-methyl-1H-1,2,4-triazol-5-yl)-3H-imidazo[4,5-b]pyridin-2-yl)pyridin-2-amine (201 mg, yield: 40% for two steps) was obtained as a brown solid. MS: m/z=513.2 [M+H]⁺. ¹H NMR (400 MHz, Dimethysulfoxide-d₆) δ 8.37 (d, J=8.4 Hz, 1H), 8.16 (d, J=8.4 Hz, 1H), 8.08-7.92 (m, 2H), 7.53-7.46 (m, 4H), 7.25 (dd, J=7.6, 1.6 Hz, 1H), 7.02 (s, 2H), 6.41 (dd, J=8.0, 4.8 Hz, 1H), 4.80 (s, 2H), 4.08 (s, 3H), 0.91 (s, 9H), 0.07 (s, 6H).

### Step 3: (4-(2-(2-Aminopyridin-3-yl)-5-(1-methyl-1H-1,2,4-triazol-5-yl)-3H-imidazo[4,5-b]pyridin-3-yl)phenyl)methanol

To a solution of 3-(3-(4-(((tert-butyldimethylsilyl)oxy)methyl)phenyl)-5-(1-methyl-1H-1,2,4-triazol-5-yl)-3H-imidazo[4,5-b]pyridin-2-yl)pyridin-2-amine (201 mg, 392 μmol) in THF (2 mL) was added TBAF (0.4 mL, 392 μmol, 1 M in THF). The mixture was stirred at 25° C. for 1 hr. The reaction mixture was poured into H₂O (15 mL), extracted with EtOAc (15 mL×3). The combined organic layers were dried over anhydrous Na₂SO₄, filtered, and concentrated under reduced pressure to give (4-(2-(2-aminopyridin-3-yl)-5-(1-methyl-1H-1,2,4-triazol-5-yl)-3H-imidazo[4,5-b]pyridin-3-yl)phenyl)methanol (155 mg, crude) as a brown solid. MS: m/z=399.1 [M+H]⁺.

### Step 4: 3-(3-(4-(Chloromethyl)phenyl)-5-(1-methyl-1H-1,2,4-triazol-5-yl)-3H-imidazo[4,5-b]pyridin-2-yl)pyridin-2-amine

To a solution of (4-(2-(2-aminopyridin-3-yl)-5-(1-methyl-1H-1,2,4-triazol-5-yl)-3H-imidazo[4,5-b]pyridin-3-yl)phenyl)methanol (155 mg, 389 μmol) in CH₂Cl₂ (2 mL) was added SOCl₂ (1 mL). The mixture was stirred at 25° C. for 1 hr. The reaction was concentrated under reduced pressure to give 3-(3-(4-(chloromethyl)phenyl)-5-(1-methyl-1H-1,2,4-triazol-5-yl)-3H-imidazo[4,5-b]pyridin-2-yl)pyridin-2-amine (Intermediate 219, 180 mg, HCl salt) as a brown solid. MS: m/z=417.0 [M+H]⁺.

### Intermediate 220: 3-(3-(4-(Chloromethyl)phenyl)-5-(4-methyl-2H-1,2,3-triazol-2-yl)-3H-imidazo[4,5-b]pyridin-2-yl)pyridin-2-amine

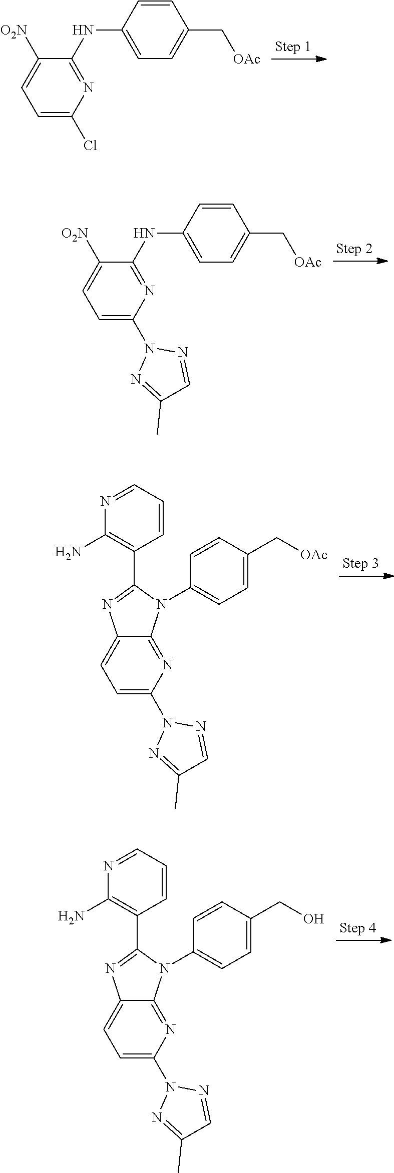 [SMILES: CC(=O)OCc1ccc(Nc2nc(Cl)ccc2[N+](=O)[O-])cc1 ; CC(=O)OCc1ccc(Nc2nc(-n3ncc(C)n3)ccc2[N+](=O)[O-])cc1 ; CC(=O)OCc1ccc(-n2c(-c3cccnc3N)nc3ccc(-n4ncc(C)n4)nc32)cc1 ; Cc1cnn(-c2ccc3nc(-c4cccnc4N)n(-c4ccc(CO)cc4)c3n2)n1]

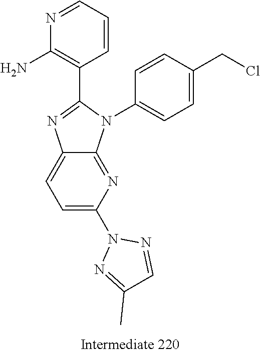 [SMILES: Cc1cnn(-c2ccc3nc(-c4cccnc4N)n(-c4ccc(CCl)cc4)c3n2)n1]

### Step 1: 4-((6-(4-Methyl-2H-1,2,3-triazol-2-yl)-3-nitropyridin-2-yl)amino)benzyl acetate

To a solution of 4-((6-chloro-3-nitropyridin-2-yl)amino)benzyl acetate (refer to Intermediate 178 for detail procedures, 3 g, 9.33 mmol) and 4-methyl-1H-1,2,3-triazole (775 mg, 9.33 mmol) in DMF (30 mL) were added K₂CO₃ (6.44 g, 46.6 mmol) and NaI (140 mg, 933 μmol). The mixture was stirred at 80° C. for 1 hr. The reaction mixture was quenched with H₂O (100 mL) at 25° C. and extracted with CH₂Cl₂ (100 mL×2). The combined organic layers were washed with brine (100 mL×3), dried over Na₂SO₄, filtered, and concentrated under reduced pressure. After purified by silica gel flash chromatography (Eluent of 10%˜20% EtOAc in petroleum ether), 4-((6-(4-methyl-2H-1,2,3-triazol-2-yl)-3-nitropyridin-2-yl)amino)benzyl acetate (1.4 g, yield: 40%) was obtained as a red solid. MS: m/z=369.1 [M+H]⁺. ¹H NMR (400 MHz, Dimethylsulfoxide-d₆) δ 10.33 (s, 1H), 8.76 (d, J=9.2 Hz, 1H), 8.13 (s, 1H), 8.03-7.95 (m, 2H), 7.50 (d, 0.1=9.2 Hz, 1H), 7.41 (d, 1=8.4 Hz, 2H), 5.08 (s, 2H), 2.42 (s, 3H), 2.08 (s, 3H).

### Step 2: 4-(2-(2-Aminopyridin-3-yl)-5-(4-methyl-2H-1,2,3-triazol-2-yl)-3H-imidazo[4,5-b]pyridin-3-yl)benzyl acetate

To a solution of 4-((6-(4-methyl-2H-1,2,3-triazol-2-yl)-3-nitropyridin-2-yl)amino)benzyl acetate (1.4 g, 3.8 mmol) in DMSO (10 mL) were added Na₂S₂O₄ (2.65 g, 15.2 mmol, 87% purity) and 2-aminonicotinaldehyde (511 mg, 4.18 mmol). The mixture was stirred at 100° C. for 4 hr. The reaction mixture was quenched with H₂O (100 mL) at 25° C. and extracted with CH₂Cl₂ (100 mL×2). The combined organic layers were washed with brine (100 mL×2), dried over Na₂SO₄, filtered, and concentrated under reduced pressure. After purified by silica gel flash chromatography (Eluent of 20%˜70% EtOAc in petroleum ether), 4-(2-(2-aminopyridin-3-yl)-5-(4-methyl-2H-1,2,3-triazol-2-yl)-3H-imidazo[4,5-b]pyridin-3-yl)benzyl acetate (240 mg, yield: 14%) was obtained as a red solid. MS: m/z=441.1 [M+H]⁺. ¹H NMR (400 MHz, Dimethylsulfoxide-d₆) δ 8.40 (d, J=8.8 Hz, 1H), 8.03-7.88 (m, 3H), 7.58-7.47 (m, 4H), 7.23 (dd, J=7.6, 1.6 Hz, 1H), 6.84 (s, 2H), 6.43 (dd, J=7.6, 4.8 Hz, 1H), 5.17 (s, 2H), 2.35 (s, 3H), 2.12 (s, 3H).

### Step 3: (4-(2-(2-Aminopyridin-3-yl)-5-(4-methyl-2H-1,2,3-triazol-2-yl)-3H-imidazo[4,5-b]pyridin-3-yl)phenyl)methanol

To a solution of 4-(2-(2-aminopyridin-3-yl)-5-(4-methyl-2H-1,2,3-triazol-2-yl)-3H-imidazo[4,5-b]pyridin-3-yl)benzyl acetate (240 mg, 545 μmol) in THF (5 mL) and MeOH (5 mL) was added K₂CO₃ (226 mg, 1.63 mmol). The mixture was stirred at 25° C. for 0.5 hr. The reaction mixture was quenched with H₂O (50 mL) at 25° C. and extracted with CH₂Cl₂ (50 mL×3). The combined organic layers were washed with brine (50 mL×2), dried over Na₂SO₄, filtered and concentrated under reduced pressure. (4-(2-(2-Aminopyridin-3-yl)-5-(4-methyl-2H-1,2,3-triazol-2-yl)-3H-imidazo[4,5-b]pyridin-3-yl)phenyl)methanol (150 mg, yield: 68%) was obtained as a red solid. MS: m/z=399.2 [M+H]⁺. ¹H NMR (400 MHz, Dimethylsulfoxide-d₆) δ 8.39 (d, J=8.4 Hz, 1H), 8.02-7.84 (m, 3H), 7.52-7.41 (m, 4H), 7.22 (dd, J=7.6, 2.0 Hz, 1H), 6.91 (s, 2H), 6.44-6.37 (m, 1H), 5.35 (t, J=5.6 Hz, 1H), 4.60 (d, J=6.0 Hz, 2H), 2.35 (s, 3H).

### Step 4: 3-(3-(4-(Chloromethyl)phenyl)-5-(4-methyl-2H-1,2,3-triazol-2-yl)-3H-imidazo[4,5-b]pyridin-2-yl)pyridin-2-amine

To a solution of (4-(2-(2-aminopyridin-3-yl)-5-(4-methyl-2H-1,2,3-triazol-2-yl)-3H-imidazo[4,5-b]pyridin-3-yl)phenyl)methanol (150 mg, 376 μmol) in CH₂Cl₂ (5 mL) was added SOCl₂ (134 mg, 1.13 mmol). The mixture was stirred at 40° C. for 0.5 hr. The reaction was concentrated under reduced pressure to give 3-(3-(4-(chloromethyl)phenyl)-5-(4-methyl-2H-1,2,3-triazol-2-yl)-3H-imidazo[4,5-b]pyridin-2-yl)pyridin-2-amine (Intermediate 220, 170 mg, HCl salt) as a green solid. MS: m/z=417.1 [M+H]⁺.

### Intermediate 221: 3-(3-(4-(Chloromethyl)phenyl)-5-(4-methyl-4H-1,2,4-triazol-3-yl)-3H-imidazo[4,5-b]pyridin-2-yl)pyridin-2-amine

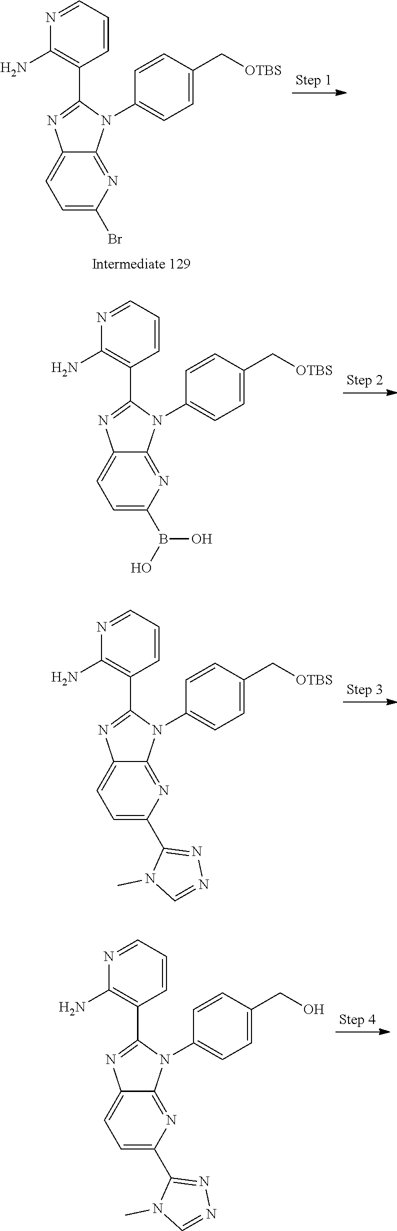

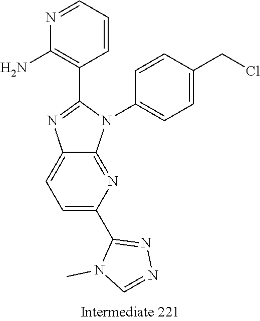 [SMILES: Cn1cnnc1-c1ccc2nc(-c3cccnc3N)n(-c3ccc(CCl)cc3)c2n1]

### Step 1: (2-(2-Aminopyridin-3-yl)-3-(4-(((tert-butyldimethylsilyl)oxy)methyl)phenyl)-3H-imidazo[4,5-h]pyridin-5-yl)boronic acid

A mixture of Intermediate 129 (1 g, 1.96 mmol), 4,4,4′,4′,5,5,5′,5′-octamethyl-2,2′-bi(1,3,2-dioxaborolane) (995 mg, 3.92 mmol), KOAc (577 mg, 5.88 mmol) and Pd(dppf)Cl₂ (143 mg, 196 mmol) in 1,4-dioxane (5 mL) was degassed and purged with N₂ three times. The mixture was stirred at 90° C. for 16 hr under N₂ atmosphere. (2-(2-Aminopyridin-3-yl)-3-(4-(((tert-butyldimethylsilyl)oxy)methyl)phenyl)-3H-imidazo[4,5-b]pyridin-5-yl)boronic acid was obtained as a black liquid and was used in the next step without work-up and purification. MS: m/z=476.1 [M+H]⁺.

### Step 2: 3-(3-(4-(((tert-Butyldimethylsilyl)oxy)methyl)phenyl)-5-(4-methyl-4H-1,2,4-triazol-3-yl)-3H₁-imidazo[4,5-h]pyridin-2-yl)pyridin-2-amine

A mixture of (2-(2-aminopyridin-3-yl)-3-(4-(((tert-butyldimethylsilyl)oxy)methyl)phenyl)-3H-imidazo[4,5-b]pyridin-5-yl)boronic acid (470 mg, 989 μmol), 3-bromo-4-methyl-4H-1,2,4-triazole (192 mg, 1.19 mmol), Pd(dppf)Cl₂ (72.3 mg, 98.9 μmol) and Cs₂CO₃ (966 mg, 2.97 mmol) in 1,4-dioxane (4 mL) and H₂O (1 mL) was degassed and purged with N₂ three times. The mixture was stirred at 90° C. for 16 hr under N₂ atmosphere. The reaction mixture was poured into H₂O (15 mL) and extracted with EtOAc (15 mL×3). The combined organic phases were dried over anhydrous Na₂SO₄, filtered and concentrated under reduced pressure. After purified by silica gel flash chromatography (Eluent of 0%˜50% EtOAc in petroleum ether), 3-(3-(4-(((tert-butyldimethylsilyl)oxy)methyl)phenyl)-5-(4-methyl-4H-1,2,4-triazol-3-yl)-3H-imidazo[4,5-b]pyridin-2-yl)pyridin-2-amine (200 mg, yield: 40% for two steps) was obtained as a brown solid. MS: m/z=513.2 [M+H]⁺. ¹H NMR (400 MHz, Dimethysulfoxide-d₆) δ 8.63 (s, 1H), 8.37 (d, J=8.4 Hz, 1H), 8.16 (d, J=8.4 Hz, 1H), 8.08-7.92 (m, 1H), 7.53-7.46 (m, 4H), 7.25 (dd, J=7.6, 1.6 Hz, 1H), 7.02 (s, 2H), 6.41 (dd, J=7.6, 4.8 Hz, 1H), 4.80 (s, 2H), 4.08 (s, 3H), 0.91 (s, 9H), 0.07 (s, 6H).

### Step 3: (4-(2-(2-Aminopyridin-3-yl)-5-(4-methyl-4H-1,2,4-triazol-3-yl)-3H-imidazo[4,5-b]pyridin-3-yl)phenyl)methanol

To a solution of 3-(3-(4-(((tert-butyldimethylsilyl)oxy)methyl)phenyl)-5-(4-methyl-4H-1,2,4-triazol-3-yl)-3H-imidazo[4,5-b]pyridin-2-yl)pyridin-2-amine (200 mg, 390 μmol) in THF (2 mL) was added TBAF (1 mL, 1 M in THF). The mixture was stirred at 25° C. for 0.5 hr. The reaction mixture was poured into H₂O (15 mL) and extracted with EtOAc (15 mL×3). The combined organic phases were dried over anhydrous Na₂SO₄, filtered and concentrated under reduced pressure. (4-(2-(2-Aminopyridin-3-yl)-5-(4-methyl-4H-1,2,4-triazol-3-yl)-3H-imidazo[4,5-b]pyridin-3-yl)phenyl)methanol (155 mg, crude) was obtained as a brown solid. MS: m/z=399.1 [M+H]⁺.

### Step 4: 3-(3-(4-(Chloromethyl)phenyl)-5-(4-methyl-4H-1,2,4-triazol-3-yl)-3H-imidazo[4,5-b]pyridin-2-yl)pyridin-2-amine

To a solution of (4-(2-(2-aminopyridin-3-yl)-5-(4-methyl-4H-1,2,4-triazol-3-yl)-3H-imidazo[4,5-b]pyridin-3-yl)phenyl)methanol (155 mg, 389 μmol) in CH₂Cl₂ (2 mL) was added SOCl₂ (1 mL). The mixture was stirred at 25° C. for 0.5 hr. The reaction was concentrated under reduced pressure, 3-(3-(4-(chloromethyl)phenyl)-5-(4-methyl-4H-1,2,4-triazol-3-yl)-3H-imidazo[4,5-b]pyridin-2-yl)pyridin-2-amine (Intermediate 221, 160 mg, HCl salt) was obtained as a brown solid. MS: m/z=417.1 [M+H]⁺.

### Intermediate 222: 3-(3-(4-(Chloromethyl)phenyl)-5-(3-fluoropyridin-2-yl)-3H-imidazo[4,5-b]pyridin-2-yl)pyridin-2-amine

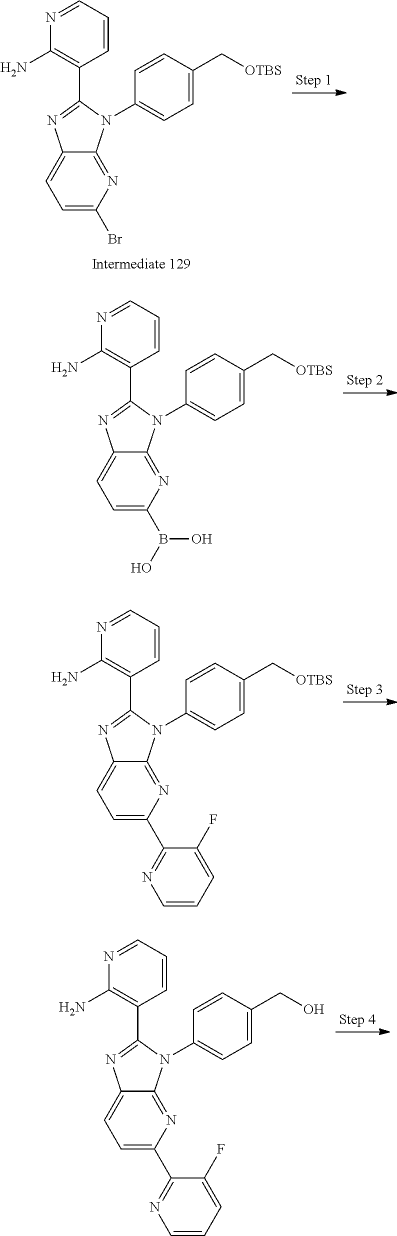

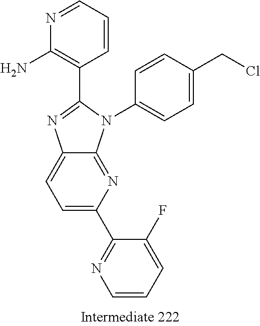 [SMILES: Nc1ncccc1-c1nc2ccc(-c3ncccc3F)nc2n1-c1ccc(CCl)cc1]

### Step 1: (2-(2-Aminopyridin-3-yl)-3-(4-(((tert-butyldimethylsilyl)oxy)methyl)phenyl)-3H-imidazo[4,5-b]pyridin-5-yl)boronic acid

To a solution of Intermediate 129 (500 mg, 0.98 mmol) and 4,4,5,5-tetramethyl-2-(4,4,5,5-tetramethyl-1,3,2-dioxaborolan-2-yl)-1,3,2-dioxaborolane (497 mg, 1.96 mmol) in 1,4-dioxane (10 mL) were added KOAc (288 mg, 2.94 mmol) and Pd(dppf)Cl₂ (71 mg, 98 μmol) at 25° C. This mixture was stirred at 80° C. for 4 hr. (2-(2-Aminopyridin-3-yl)-3-(4-(((tert-butyldimethylsilyl)oxy)methyl)phenyl)-3H-imidazo[4,5-b]pyridin-5-yl)boronic acid was obtained as black liquid, which was used in the next step directly. MS: m/z=476.2 [M+H]⁺.

### Step 2: 3-(3-(4-(((tert-Butyldimethylsilyl)oxy)methyl)phenyl)-5-(3-fluoropyridin-2-yl)-3H-imidazo[4,5-b]pyridin-2-yl)pyridin-2-amine

To a mixture of (2-(2-aminopyridin-3-yl)-3-(4-(((tert-butyldimethylsilyl)oxy)methyl)phenyl)-3H-imidazo[4,5-b]pyridin-5-yl)boronic acid (450 mg, 946 μmol) and 2-bromo-3-fluoropyridine (183 mg, 1.04 mmol) in 1,4-dioxane (5 mL) and H₂O (1 mL) were added Cs₂CO₃ (925 mg, 2.84 mmol) and Pd(dppf)Cl₂ (69.3 mg, 94.7 μmol). The mixture was stirred at 100° C. for 2 hr under N₂. The mixture was diluted with H₂O (50 mL) and extracted with EtOAc (50 mL×2). The combined organic layers were washed with brine (50 mL), dried over anhydrous Na₂SO₄, filtered and concentrated. After purified by silica gel flash chromatography (EtOAc in petroleum ether=0-50%), 3-(3-(4-(((tert-butyldimethylsilyl)oxy)methyl)phenyl)-5-(3-fluoropyridin-2-yl)-3H-imidazo[4,5-b]pyridin-2-yl)pyridin-2-amine (300 mg, yield: 54% for two steps) was obtained as a brown solid. MS: m/z=527.3 [M+H]⁺. ¹H NMR (400 MHz, Dimethylsulfoxide-d₆) δ 8.57-8.50 (m, 1H), 8.33 (d, J=8.4 Hz, 1H), 8.03-7.98 (m, 2H), 7.86-7.76 (m, 1H), 7.57-7.49 (m, 1H), 7.47-7.41 (m, 4H), 7.21 (dd, J=7.6, 1.6 Hz, 1H), 7.02 (s, 2H), 6.39 (dd, J=7.6, 4.8 Hz, 1H), 4.80 (s, 2H), 0.91 (s, 9H), 0.10 (s, 6H).

### Step 3: (4-(2-(2-Aminopyridin-3-yl)-5-(3-fluoropyridin-2-yl)-3H-imidazo[4,5-b]pyridin-3-yl)phenyl)methanol

To a mixture of 3-(3-(4-(((tert-butyldimethylsilyl)oxy)methyl)phenyl)-5-(3-fluoropyridin-2-yl)-3H-imidazo[4,5-b]pyridin-2-yl)pyridin-2-amine (250 mg, 475 μmol) in THF (2 mL) was added TBAF (1 M in THF, 712 μL). The mixture was stirred at 20° C. for 0.5 hr. The mixture was diluted with H₂O (20 mL) and extracted with EtOAc (20 mL×2). The combined organic layers were washed with brine (20 mL), dried over anhydrous Na₂SO₄, filtered and concentrated. (4-(2-(2-Aminopyridin-3-yl)-5-(3-fluoropyridin-2-yl)-3H-imidazo[4,5-b]pyridin-3-yl)phenyl)methanol (195 mg, yield: 99%) was obtained as a brown solid. MS: m/z=413.2 [M+H]⁺. ¹H NMR (400 MHz, Dimethylsulfoxide-d₆) δ 8.56-8.50 (m, 1H), 8.33 (d, J=8.0 Hz, 1H), 8.03-7.97 (m, 2H), 7.85-7.76 (m, 1H), 7.56-7.50 (m, 1H), 7.47-7.41 (m, 4H), 7.25 (dd, J=7.6, 1.6 Hz, 1H), 6.94 (s, 2H), 6.43 (dd, J=7.6, 4.8 Hz, 1H), 5.34 (t, J=6.0 Hz, 1H), 4.58 (d, J=5.6 Hz, 2H).

### Step 4: 3-(3-(4-(Chloromethyl)phenyl)-5-(3-fluoropyridin-2-yl)-3H-imidazo[4,5-b]pyridin-2-yl)pyridin-2-amine

To a mixture of (4-(2-(2-aminopyridin-3-yl)-5-(3-fluoropyridin-2-yl)-3H-imidazo[4,5-b]pyridin-3-yl)phenyl)methanol (195 mg, 472 μmol) in CH₂Cl₂ (3 mL) was added SOCl₂ (169 mg, 1.42 mmol). The mixture was stirred at 20° C. for 1 hr. The mixture was concentrated to give 3-(3-(4-(chloromethyl)phenyl)-5-(3-fluoropyridin-2-yl)-3H-imidazo[4,5-b]pyridin-2-yl)pyridin-2-amine (Intermediate 222, 203 mg, yield: 99%) as a brown solid. MS: m/z=431.1 [M+H]⁺.

### Intermediate 223: 3-(3-(4-(Chloromethyl)phenyl)-5-(6-methylpyridin-2-yl)-3H-imidazo[4,5-b]pyridin-2-yl)pyridin-2-amine

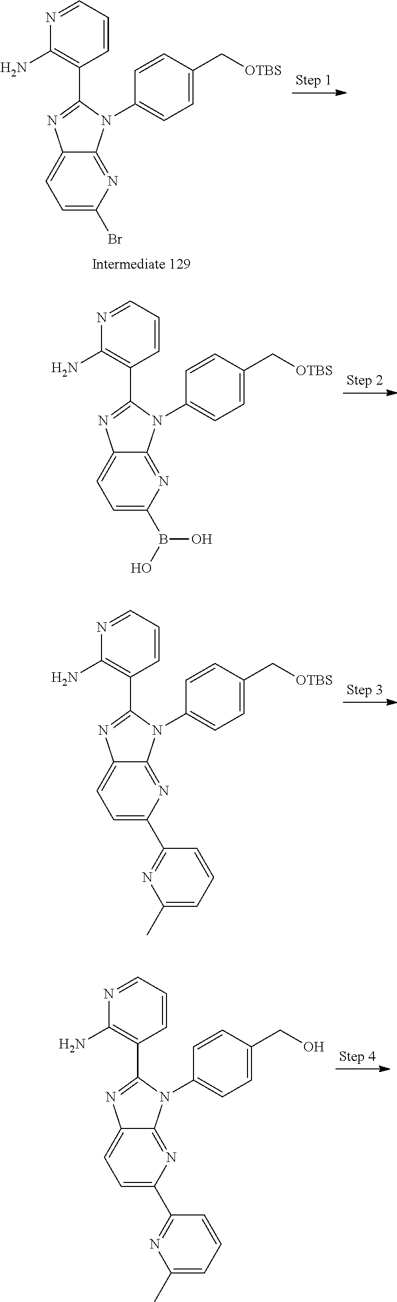

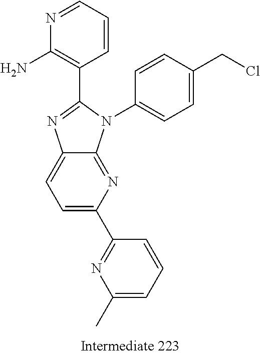 [SMILES: Cc1cccc(-c2ccc3nc(-c4cccnc4N)n(-c4ccc(CCl)cc4)c3n2)n1]

### Step 1: (2-(2-Aminopyridin-3-yl)-3-(4-(((tert-butyldimethylsilyl)oxy)methyl)phenyl)-3H-imidazo[4,5-b]pyridin-5-yl)boronic acid

To a solution of Intermediate 129 (500 mg, 0.98 mmol) and 4,4,5,5-tetramethyl-2-(4,4,5,5-tetramethyl-1,3,2-dioxaborolan-2-yl)-1,3,2-dioxaborolane (497 mg, 1.96 mmol) in 1,4-dioxane (10 mL) were added KOAc (288 mg, 2.94 mmol) and Pd(dppf)Cl₂ (71 mg, 98 μmol) at 25° C. This mixture was stirred at 80° C. for 4 hr. (2-(2-Aminopyridin-3-yl)-3-(4-(((tert-butyldimethylsilyl)oxy)methyl)phenyl)-3H-imidazo[4,5-b]pyridin-5-yl)boronic acid was obtained as black liquid, which was used in the next step directly. MS: m/z=476.2 [M+H]⁺

### Step 2: 3-(3-(4-(((tert-Butyldimethylsilyl)oxy)methyl)phenyl)-5-(6-methylpyridin-2-yl)-3H-imidazo[4,5-b]pyridin-2-yl)pyridin-2-amine

To a mixture of (2-(2-aminopyridin-3-yl)-3-(4-(((tert-butyldimethylsilyl)oxy)methyl)phenyl)-3H-imidazo[4,5-b]pyridin-5-yl)boronic acid (450 mg, 947 μmol) and 2-bromo-6-methylpyridine (179 mg, 1.04 mmol) in 1,4-dioxane (5 mL) and H₂O (1 mL) were added Cs₂CO₃ (925 mg, 2.84 mmol) and Pd(dppf)Cl₂ (69.3 mg, 94.7 μmol). The mixture was stirred at 100° C. for 2 hr under N₂. The mixture was diluted with H₂O (50 mL) and extracted with EtOAc (100 mL×2). The combined organic layers were washed with brine (100 mL), dried over anhydrous Na₂SO₄, filtered and concentrated. After purified by silica gel flash chromatography (EtOAc in petroleum ether=0-50%), 3-(3-(4-(((tert-butyldimethylsilyl)oxy)methyl)phenyl)-5-(6-methylpyridin-2-yl)-3H-imidazo[4,5-b]pyridin-2-yl)pyridin-2-amine (350 mg, yield: 63% for two steps) was obtained as a brown solid. MS: m/z=523.2 [M+H]⁺. ¹H NMR (400 MHz, Dimethylsulfoxide-d₆) δ 8.47 (d, J=8.4 Hz, 1H), 8.29 (d, J=8.4 Hz, 1H), 8.05-7.96 (m, 2H), 7.75 (t, J=8.0 Hz, 1H), 7.54-7.46 (m, 4H), 7.29-7.18 (m, 2H), 7.02 (s, 2H), 6.39 (dd, J=7.6, 4.8 Hz, 1H), 4.83 (s, 2H), 2.56 (s, 3H), 0.93 (s, 9H), 0.11 (s, 6H).

### Step 3: (4-(2-(2-Aminopyridin-3-yl)-5-(6-methylpyridin-2-yl)-3H-imidazo[4,5-b]pyridin-3-yl)phenyl)methanol

To a mixture of 3-(3-(4-((tert-butyldimethylsilyl)oxy)methyl)phenyl)-5-(6-methylpyridin-2-yl)-3H-imidazo[4,5-b]pyridin-2-yl)pyridin-2-amine (300 mg, 574 μmol) in THF (2 mL) was added TBAF (1 M in THF, 0.86 mL). The mixture was stirred at 20° C. for 0.5 hr. The mixture was diluted with H₂O (20 mL) and extracted with EtOAc (20 mL×2). The combined organic layers were washed with brine (20 mL), dried over anhydrous Na₂SO₄, filtered and concentrated. (4-(2-(2-Aminopyridin-3-yl)-5-(6-methylpyridin-2-yl)-3H-imidazo[4,5-b]pyridin-3-yl)phenyl)methanol (234 mg, yield: 99%) was obtained as a brown solid. MS: m/z=409.2 [M+H]⁺. ¹H NMR (400 MHz, Dimethylsulfoxide-d₆) δ 8.48 (d, J=8.4 Hz, 1H), 8.29 (d, J=8.4 Hz, 1H), 8.04-7.94 (m, 2H), 7.77 (t, J=7.6 Hz, 1H), 7.54-7.43 (m, 4H), 7.29-7.22 (m, 2H), 6.96 (s, 2H), 6.43 (dd, J=7.6, 4.8 Hz, 1H), 5.37 (t, J=5.6 Hz, 1H), 4.61 (d, J=5.6 Hz, 2H), 2.57 (s, 3H).

### Step 4: 3-(3-(4-(Chloromethyl)phenyl)-5-(6-methylpyridin-2-yl)-3H-imidazo[4,5-b]pyridin-2-yl)pyridin-2-amine

To a mixture of (4-(2-(2-aminopyridin-3-yl)-5-(6-methylpyridin-2-yl)-3H-imidazo[4,5-b]pyridin-3-yl)phenyl)methanol (230 mg, 563 μmol) in CH₂Cl₂ (3 mL) was added SOCl₂ (201 mg, 1.69 mmol). The mixture was stirred at 20° C. for 1 hr and concentrated to give 3-(3-(4-(chloromethyl)phenyl)-5-(6-methylpyridin-2-yl)-3H-imidazo[4,5-b]pyridin-2-yl)pyridin-2-amine (Intermediate 223, 240 mg HCl salt, yield: 99%) as a brown solid. MS: m/z=427.2 [M+H]⁺.

### Intermediate 224: 3-(3-(4-(Chloromethyl)phenyl)-5-(1-(difluoromethyl)-1H-1,2,4-triazol-3-yl)-3H-imidazo[4,5-b]pyridin-2-yl)pyridin-2-amine

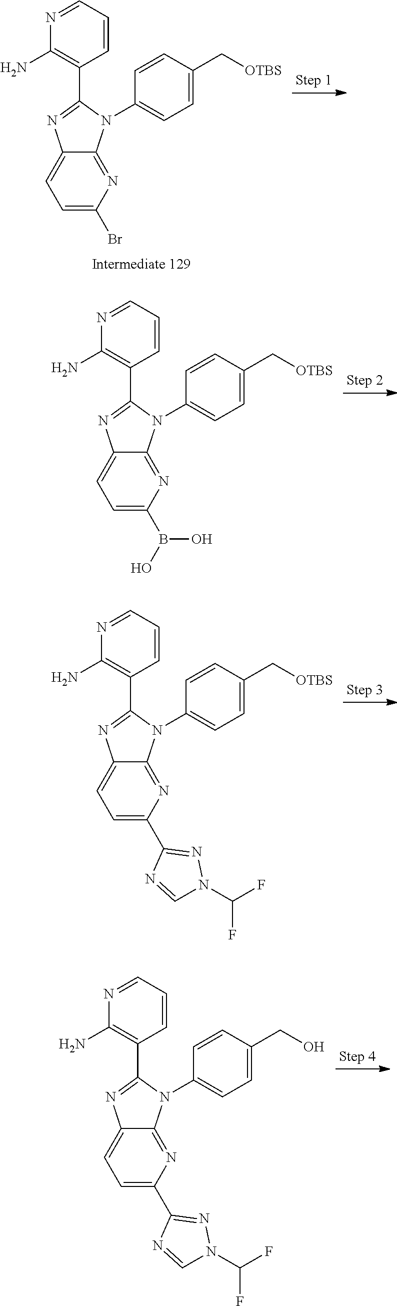

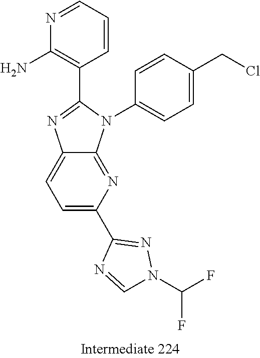 [SMILES: Nc1ncccc1-c1nc2ccc(-c3ncn(C(F)F)n3)nc2n1-c1ccc(CCl)cc1]

### Step 1: (2-(2-Aminopyridin-3-yl)-3-(4-(((tert-butyldimethylsilyl)oxy)methyl)phenyl)-3H-imidazo[4,5-b]pyridin-5-yl)boronic acid

A mixture of Intermediate 129 (400 mg, 784 μmol), 4,4,4′,4′,5,5,5′,5′-octamethyl-2,2′-bi(1,3,2-dioxaborolane) (398 mg, 1.57 mmol), KOAc (231 mg, 2.35 mmol) and Pd(dppf)Cl₂ (57.3 mg, 78.4 μmol) in 1,4-dioxane (5 mL) was degassed and purged with N₂ three times. The mixture was stirred at 80° C. for 2 hr under N₂ atmosphere. (2-(2-aminopyridin-3-yl)-3-(4-(((tert-butyldimethylsilyl)oxy)methyl)phenyl)-3H-imidazo[4,5-b]pyridin-5-yl)boronic acid was obtained as a black liquid, which was used in the next step without work-up and purification. MS: m/z=476.1 [M+H]⁺.

### Step 2: 3-(3-(4-(((tert-Butyldimethylsilyl)oxy)methyl)phenyl)-5-(1-(difluoromethyl)-1H-1,2,4-triazol-3-yl)-3H-imidazo[4,5-b]pyridin-2-yl)pyridin-2-amine

A mixture of (2-(2-aminopyridin-3-yl)-3-(4-(((tert-butyldimethylsilyl)oxy)methyl)phenyl)-3H-imidazo[4,5-b]pyridin-5-yl)boronic acid (372 mg, 782 μmol), 3-bromo-1-(difluoromethyl)-1H-1,2,4-triazole (186 mg, 939 μmol), Pd(dppf)Cl₂ (57.3 mg, 78.3 μmol) and Cs₂CO₃ (165 mg, 2.35 mmol) in 1,4-dioxane (5 mL) and H₂O (1 mL) was degassed and purged with N₂ three times. The mixture was stirred at 80° C. for 16 hr under N₂ atmosphere. The reaction mixture was quenched with H₂O (50 mL) and extracted with CH₂Cl₂ (50 mL×2). The combined organic layers were washed with brine (50 mL), dried over Na₂SO₄, filtered, and concentrated under reduced pressure. After purified by silica gel flash chromatography (Eluent of 10%˜50% EtOAc in petroleum ether), 3-(3-(4-(((tert-butyldimethylsilyl)oxy)methyl)phenyl)-5-(1-(difluoromethyl)-1H-1,2,4-triazol-3-yl)-3H-imidazo[4,5-b]pyridin-2-yl)pyridin-2-amine (220 mg, yield: 37% for two steps) was obtained as a yellow solid. MS: m/z=549.2 [M+H]⁺. ¹H NMR (400 MHz, Dimethylsulfoxide-d₆) S 9.18 (s, 1H), 8.36-8.28 (m, 1H), 8.18 (d, J=8.0 Hz, 1H), 8.04-7.82 (m, 2H), 7.50-7.35 (m, 4H), 7.25-7.17 (m, 1H), 7.04-6.89 (m, 2H), 6.46-6.32 (m, 1H), 4.83 (s, 2H), 0.93 (s, 9H), 0.12 (s, 6H).

### Step 3: (4-(2-(2-Aminopyridin-3-yl)-5-(1-(difluoromethyl)-1H-1,2,4-triazol-3-yl)-3H-imidazo[4,5-b]pyridin-3-yl)phenyl)methanol

To a solution of 3-(3-(4-(((tert-butyldimethylsilyl)oxy)methyl)phenyl)-5-(1-(difluoromethyl)-1H-1,2,4-triazol-3-yl)-3H-imidazo[4,5-b]pyridin-2-yl)pyridin-2-amine (220 mg, 401 μmol) in THF (5 mL) was added TBAF (0.8 mL, 1 M in THF). The mixture was stirred at 25° C. for 0.5 hr. The reaction mixture was quenched with H₂O (20 mL) at 25° C. and extracted with CH₂Cl₂ (20 mL×3). The combined organic layers were washed with brine (20 mL×2), dried over Na₂SO₄, filtered and concentrated under reduced pressure. After purified by silica gel flash chromatography (Eluent of 50%˜100% EtOAc in petroleum ether), (4-(2-(2-aminopyridin-3-yl)-5-(1-(difluoromethyl)-1H-1,2,4-triazol-3-yl)-3H-imidazo[4,5-b]pyridin-3-yl)phenyl)methanol (50 mg, yield: 26%) was obtained as a yellow solid. MS: m/z=435.1 [M+H]⁺. ¹H NMR (400 MHz, Dimethylsulfoxide-d₆) δ 9.19 (s, 1H), 8.34 (d, J=8.4 Hz, 1H), 8.19 (d, J=8.0 Hz, 1H), 8.07-7.77 (m, 2H), 7.54-7.41 (m, 4H), 7.24 (d, J=7.2 Hz, 1H), 6.97 (s, 2H), 6.46-6.34 (m, 1H), 5.37 (t, J=5.6 Hz, 1H), 4.61 (d, J=5.2 Hz, 2H).

### Step 4: 3-(3-(4-(Chloromethyl)phenyl)-5-(1-(difluoromethyl)-1H-1,2,4-triazol-3-yl)-3H-imidazo[4,5-b]pyridin-2-yl)pyridin-2-amine

To a solution of (4-(2-(2-aminopyridin-3-yl)-5-(1-(difluoromethyl)-1H-1,2,4-triazol-3-yl)-3H-imidazo[4,5-b]pyridin-3-yl)phenyl)methanol (50 mg, 115 μmol) in CH₂Cl₂ (5 mL) was added SOCl₂ (41.1 mg, 345 μmol). The mixture was stirred at 25° C. for 0.5 hr. The reaction was concentrated under reduced pressure to give 3-(3-(4-(chloromethyl)phenyl)-5-(1-(difluoromethyl)-1H-1,2,4-triazol-3-yl)-3H-imidazo[4,5-b]pyridin-2-yl)pyridin-2-amine (Intermediate 224, 52 mg, HCl slat) as a yellow solid. MS: m/z=453.0 [M+H]⁺.

### Intermediate 225: 4-((5′-Fluoro-5-nitro-[2,2′-bipyridin]-6-yl)amino)benzyl acetate

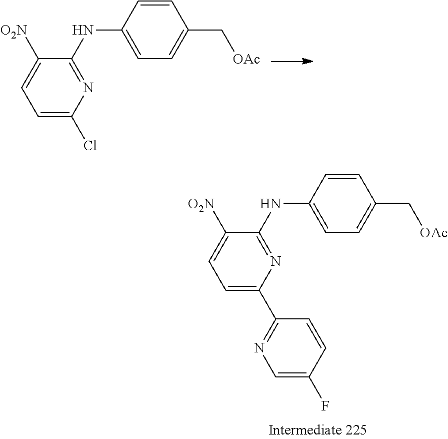 [SMILES: CC(=O)OCc1ccc(Nc2nc(Cl)ccc2[N+](=O)[O-])cc1 ; CC(=O)OCc1ccc(Nc2nc(-c3ccc(F)cn3)ccc2[N+](=O)[O-])cc1]

A mixture of 4-((6-chloro-3-nitropyridin-2-yl)amino)benzyl acetate (refer to Intermediate 178 for detail procedures, 10 g, 31 mmol), (5-fluoropyridin-2-yl)boronic acid (13 g, 93 mmol), Cata CXium A Pd G3 (2.3 g, 3.1 mmol), CuBr (3.34 g, 23.3 mmol) and Cs₂CO₃ (30 g, 93 mmol) in 1,4-dioxane (350 mL) was degassed and purged with N₂ three times. The mixture was stirred at 125° C. for 2.5 hr under N₂ atmosphere. The reaction mixture was poured into H₂O (500 mL) and extracted with CH₂Cl₂ (200 mL×2). The combined organic layers were washed with brine (500 mL), dried over Na₂SO₄, filtered, and concentrated under reduced pressure. After triturated (10% EtOAc in petroleum ether, 100 mL) at 25° C. for 1 hr, 4-((5′-fluoro-5-nitro-[2,2′-bipyridin]-6-yl)amino)benzyl acetate (Intermediate 225, 10 g, yield: 72%) was obtained as a yellow solid. MS: m/z=383.0 [M+H]⁺. ¹H NMR (400 MHz, Dimethylsulfoxide-d₆) δ 10.10 (s, 1H), 8.77-8.74 (m, 1H), 8.69 (d, J=8.8 Hz, 1H), 8.25-8.21 (m, 1H), 7.97-7.92 (m, 1H), 7.90 (d, J=8.4 Hz, 1H), 7.75 (d, J=8.4 Hz, 2H), 7.48-7.44 (m, 2H), 5.10 (s, 2H), 2.09 (s, 3H). ¹⁹F NMR (400 MHz, Dimethylsulfoxide-d₆) δ −124.159.

### Intermediate 226: 5-(3-(4-(Chloromethyl)phenyl)-5-(5-fluoropyridin-2-yl)-3H-imidazo[4,5-b]pyridin-2-yl)pyrimidin-4-amine

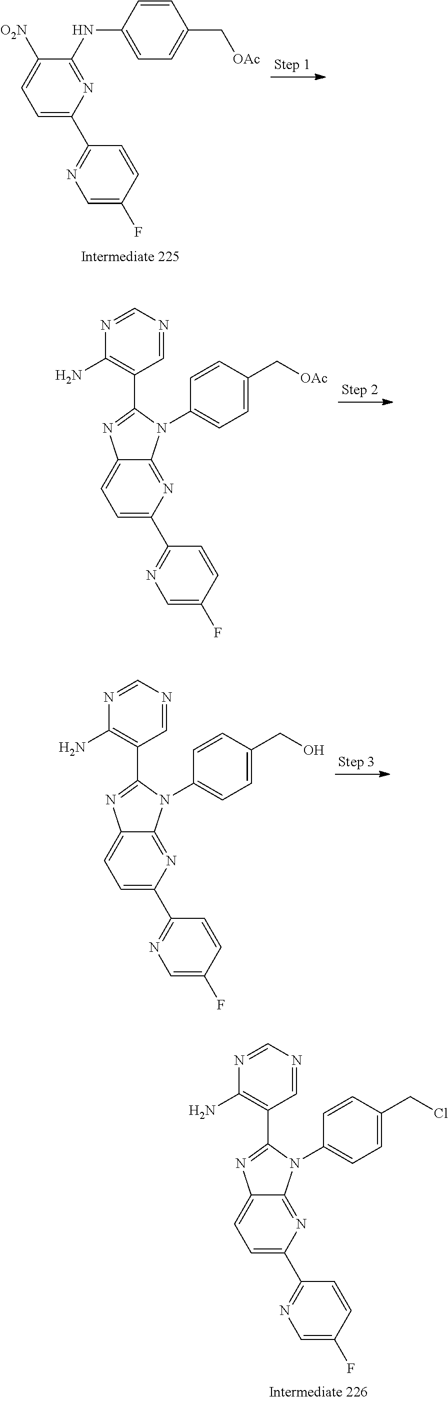 [SMILES: CC(=O)OCc1ccc(Nc2nc(-c3ccc(F)cn3)ccc2[N+](=O)[O-])cc1 ; CC(=O)OCc1ccc(-n2c(-c3cncnc3N)nc3ccc(-c4ccc(F)cn4)nc32)cc1 ; Nc1ncncc1-c1nc2ccc(-c3ccc(F)cn3)nc2n1-c1ccc(CO)cc1 ; Nc1ncncc1-c1nc2ccc(-c3ccc(F)cn3)nc2n1-c1ccc(CCl)cc1]

### Step 1: 4-(2-(4-Aminopyrimidin-5-yl)-5-(5-fluoropyridin-2-yl)-3H-imidazo[4,5-b]pyridin-3-yl)benzyl acetate

To a solution of Intermediate 225 (2 g, 5.23 mmol) in DMSO (10 mL) were added Na₂S₂O₄ (3.64 g, 20.9 mmol, 87% purity) and 4-aminopyrimidine-5-carbaldehyde (708 mg, 5.75 mmol). The mixture was stirred at 100° C. for 2 hr. The reaction mixture was quenched with H₂O (100 mL) at 25° C. and extracted with CH₂Cl₂ (100 mL×2). The combined organic layers were washed with brine (100 mL×2), dried over Na₂SO₄, filtered, and concentrated under reduced pressure. After purified by silica gel flash chromatography (Eluent of 20%˜70% EtOAc in petroleum ether), 4-(2-(4-aminopyrimidin-5-yl)-5-(5-fluoropyridin-2-yl)-3H-imidazo[4,5-b]pyridin-3-yl)benzyl acetate (500 mg, yield: 21%) was obtained as a yellow solid. MS: m/z=456.1 [M+H]⁺. ¹H NMR (400 MHz, Dimethylsulfoxide-d₆) δ 8.67 (d, J=2.8 Hz, 1H), 8.46-8.30 (m, 3H), 8.26-8.20 (m, 1H), 8.02 (s, 1H), 7.87-7.80 (m, 1H), 7.69 (s, 2H), 7.59 (s, 4H), 5.20 (s, 2H), 2.13 (s, 3H). ¹⁹F NMR (400 MHz, Dimethylsulfoxide-d₆) δ −127.656.

### Step 2: (4-(2-(4-Aminopyrimidin-5-yl)-5-(5-fluoropyridin-2-yl)-3H-imidazo[4,5-b]pyridin-3-yl)phenyl)methanol

To a solution of 4-(2-(4-aminopyrimidin-5-yl)-5-(5-fluoropyridin-2-yl)-3H-imidazo[4,5-b]pyridin-3-yl)benzyl acetate (500 mg, 1.1 mmol) in THF (5 mL) and MeOH (5 mL) was added K₂CO₃ (455 mg, 3.29 mmol). The mixture was stirred at 25° C. for 0.5 hr. The reaction mixture was quenched with H₂O (50 mL) at 25° C. and extracted with CH₂Cl₂ (50 mL×3). The combined organic layers were washed with brine (50 mL×2), dried over Na₂SO₄, filtered and concentrated under reduced pressure. (4-(2-(4-Aminopyrimidin-5-yl)-5-(5-fluoropyridin-2-yl)-3H-imidazo[4,5-b]pyridin-3-yl)phenyl)methanol (450 mg, yield: 95%) was obtained as a yellow solid. MS: m/z=414.1 [M+H]⁺.

### Step 3: 5-(3-(4-(Chloromethyl)phenyl)-5-(5-fluoropyridin-2-yl)-3H-imidazo[4,5-b]pyridin-2-yl)pyrimidin-4-amine

To a solution of (4-(2-(4-aminopyrimidin-5-yl)-5-(5-fluoropyridin-2-yl)-3H-imidazo[4,5-b]pyridin-3-yl)phenyl)methanol (450 mg, 1.09 mmol) in CH₂Cl₂ (5 mL) was added SOCl₂ (389 mg, 3.27 mmol). The mixture was stirred at 25° C. for 0.5 hr. The reaction was concentrated under reduced pressure to give 5-(3-(4-(chloromethyl)phenyl)-5-(5-fluoropyridin-2-yl)-3H-imidazo[4,5-b]pyridin-2-yl)pyrimidin-4-amine (Intermediate 226, 509 mg, HCl salt) as a yellow solid. MS: m/z=432.0 [M+H]⁺.

### Intermediate 227: 3-(3-(4-(Chloromethyl)phenyl)-5-(5-fluoropyridin-2-yl)-3H-imidazo[4,5-b]pyridin-2-yl)pyrazin-2-amine

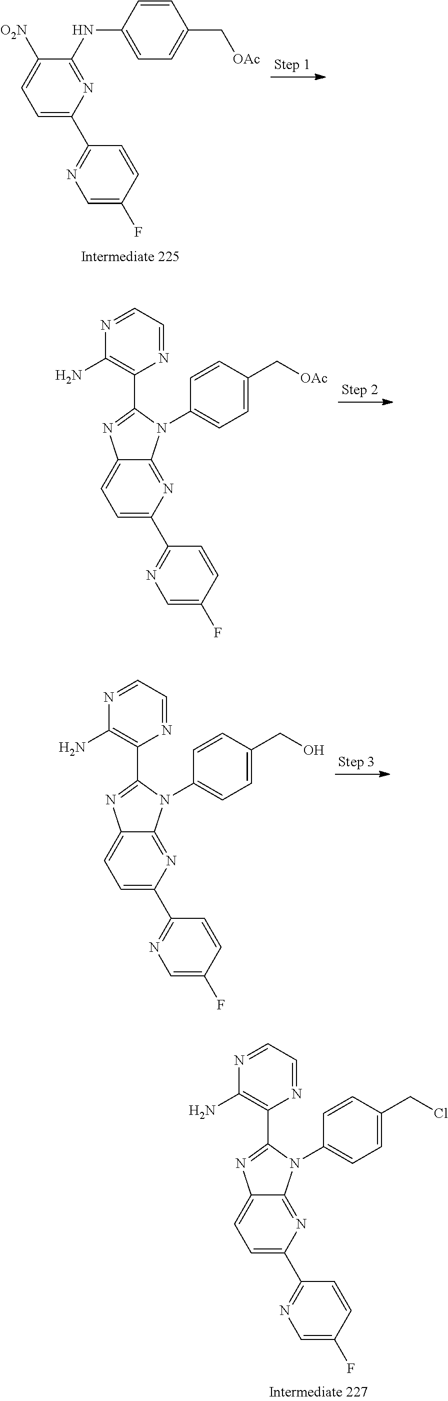 [SMILES: CC(=O)OCc1ccc(Nc2nc(-c3ccc(F)cn3)ccc2[N+](=O)[O-])cc1 ; CC(=O)OCc1ccc(-n2c(-c3nccnc3N)nc3ccc(-c4ccc(F)cn4)nc32)cc1 ; Nc1nccnc1-c1nc2ccc(-c3ccc(F)cn3)nc2n1-c1ccc(CO)cc1 ; Nc1nccnc1-c1nc2ccc(-c3ccc(F)cn3)nc2n1-c1ccc(CCl)cc1]

### Step 1: 4-(2-(3-Aminopyrazin-2-yl)-5-(5-fluoropyridin-2-yl)-3H-imidazo[4,5-b]pyridin-3-yl)benzyl acetate

A mixture of Intermediate 225 (1 g, 2.6 mmol) and 3-aminopyrazine-2-carbaldehyde (386 mg, 3.1 mmol) in DMSO (20 mL) was added Na₂S₂O₄ (1.8 g, 10.5 mmol) at 25° C. The mixture was stirred at 100° C. for 12 hr. The reaction mixture was poured into H₂O (50 mL) and extracted with CH₂Cl₂ (20 mL). The combined organic layers were washed with brine (50 mL), dried over Na₂SO₄, filtered, and concentrated under reduced pressure. After purified by silica gel flash chromatography (Eluent of 0%˜3% MeOH in CH₂Cl₂), 4-(2-(3-Aminopyrazin-2-yl)-5-(5-fluoropyridin-2-yl)-3H-imidazo[4,5-b]pyridin-3-yl)benzyl acetate (350 mg, yield: 20%) was obtained as a yellow solid. MS: m/z=456.0 [M+H]⁺. ¹H NMR (400 MHz, Dimethylsulfoxide-d₆) δ 8.67 (d, J=2.8 Hz, 1H), 8.45-8.42 (m, 1H), 8.40-8.37 (m, 1H), 8.20 (dd, J=8.8, 4.8 Hz, 1H), 8.03 (d, J=2.4 Hz, 1H), 7.88-7.82 (m, 2H), 7.54 (d, J=2.4 Hz, 1H), 7.52-7.50 (m, 2H), 7.48 (s, 2H), 7.47-7.45 (m, 1H), 5.21 (s, 2H), 2.14 (s, 3H). ¹⁹F NMR (400 MHz, Dimethylsulfoxide-d₆) δ-127.529.

### Step 2: (4-(2-(3-Aminopyrazin-2-yl)-5-(5-fluoropyridin-2-yl)-3H-imidazo[4,5-b]pyridin-3-yl)phenyl)methanol

To a solution of 4-(2-(3-aminopyrazin-2-yl)-5-(5-fluoropyridin-2-yl)-3H-imidazo[4,5-b]pyridin-3-yl)benzyl acetate (250 mg, 549 μmol) in THF (2 mL) were added K₂CO₃ (228 mg, 1.65 mmol), MeOH (2 mL) and H₂O (2 mL). The mixture was stirred at 25° C. for 1 hr. The reaction mixture was diluted with H₂O (10 mL) and extracted with CH₂Cl₂ (10 mL×3). The combined organic layers were washed with brine (30 mL), dried over Na₂SO₄, filtered and concentrated under reduced pressure. (4-(2-(3-Aminopyrazin-2-yl)-5-(5-fluoropyridin-2-yl)-3H-imidazo[4,5-b]pyridin-3-yl)phenyl)methanol (210 mg, crude) was obtained as a yellow solid. MS: m/z=414.2 [M+H]⁺.

### Step 3: 3-(3-(4-(Chloromethyl)phenyl)-5-(5-fluoropyridin-2-yl)-3H-imidazo[4,5-b]pyridin-2-yl)pyrazin-2-amine

To a solution of (4-(2-(3-aminopyrazin-2-yl)-5-(5-fluoropyridin-2-yl)-3H-imidazo[4,5-b]pyridin-3-yl)phenyl)methanol (210 mg, 508 μmol) in CH₂Cl₂ (5 mL) was added SOCl₂ (0.5 mL). The mixture was stirred at 40° C. for 1 hr. The mixture was concentrated under reduced pressure to give 3-(3-(4-(chloromethyl)phenyl)-5-(5-fluoropyridin-2-yl)-3H-imidazo[4,5-b]pyridin-2-yl)pyrazin-2-amine (Intermediate 227, 240 mg, HCl salt, crude) as a yellow solid. MS: m/z=432.0 [M+H]⁺.

### Intermediate 228: 3-(4-(Chloromethyl)phenyl)-5-(5-fluoropyridin-2-yl)-2-(pyridin-3-yl)-3H-imidazo[4,5-b]pyridine

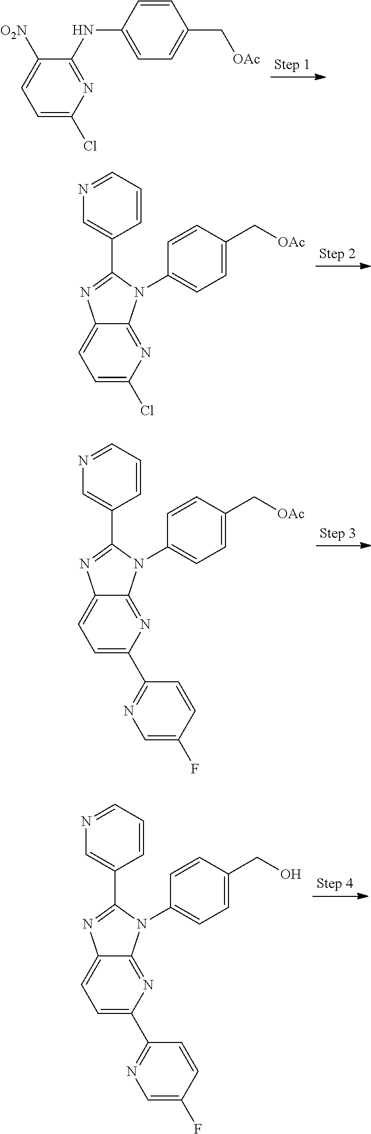

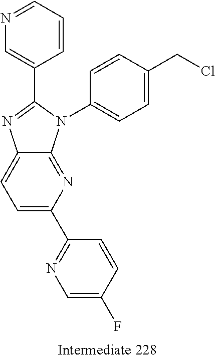 [SMILES: Fc1ccc(-c2ccc3nc(-c4cccnc4)n(-c4ccc(CCl)cc4)c3n2)nc1]

### Step 1: 4-(5-Chloro-2-(pyridin-3-yl)-3H-imidazo[4,5-b]pyridin-3-yl)benzyl acetate

A mixture of 4-((6-chloro-3-nitropyridin-2-yl)amino)benzyl acetate (refer to Intermediate 178 for detail procedures, 2 g, 6.22 mmol) and nicotinaldehyde (799 mg, 7.46 mmol) in DMSO (20 mL) was added Na₂S₂O₄ (5.1 g, 24.9 mmol) at 25° C. The mixture was stirred at 80° C. for 12 hr. The reaction mixture was poured into H₂O (50 mL) and extracted with CH₂Cl₂ (30 mL×3). The combined organic layers were washed with brine (30 mL×3), dried over Na₂SO₄, filtered, and concentrated under reduced pressure. After purified by silica gel flash chromatography (Eluent of 20%˜50% EtOAc in petroleum ether), 4-(5-chloro-2-(pyridin-3-yl)-3H-imidazo[4,5-b]pyridin-3-yl)benzyl acetate (1.2 g, yield: 51%) was obtained as a yellow solid. MS: m/z=379.1 [M+H]⁺. ¹H NMR (400 MHz, Dimethylsulfoxide-d₆) δ 8.70 (d, J=1.6 Hz, 1H), 8.63 (d, J=4.8 Hz, 1H), 8.33 (d, J=8.4 Hz, 1H), 7.91 (d, J=8.0 Hz, 1H), 7.59-7.54 (m, 2H), 7.53-7.46 (m, 4H), 5.18 (s, 2H), 2.12 (s, 3H)

### Step 2: 4-(5-(5-Fluoropyridin-2-yl)-2-(pyridin-3-yl)-3H-imidazo[4,5-b]pyridin-3-yl)benzyl acetate

A mixture of 4-(5-chloro-2-(pyridin-3-yl)-3H-imidazo[4,5-b]pyridin-3-yl)benzyl acetate (640 mg, 1.69 mmol) and (5-fluoropyridin-2-yl)boronic acid (714 mg, 5.07 mmol) in 1,4-dioxane (10 mL) were added cataCXium A Pd G₃ (123 mg, 169 μmol), CuBr (182 mg, 1.27 mmol) and Cs₂CO₃ (1.65 g, 5.07 mmol) was degassed and purged with N₂ three times. The mixture was stirred at 90° C. for 2 hr under N₂ atmosphere. The reaction mixture was poured into H₂O (30 mL) and extracted with CH₂Cl₂ (30 mL×3). The combined organic layers were washed with brine (30 mL×5), dried over Na₂SO₄, filtered, and concentrated under reduced pressure. After triturated with EtOAc (5 mL) at 25° C. for 30 min, 4-(5-(5-fluoropyridin-2-yl)-2-(pyridin-3-yl)-3H-imidazo[4,5-b]pyridin-3-yl)benzyl acetate (700 mg, yield: 94%) was obtained as a yellow solid. MS: m/z=440.1 [M+H]⁺. ¹H NMR (400 MHz, Dimethylsulfoxide-d₆) δ 8.76-8.59 (m, 3H), 8.47-8.33 (m, 2H), 8.24 (dd, J=8.8, 4.4 Hz, 1H), 7.94 (d, J=8.0 Hz, 1H), 7.88-7.80 (m, 1H), 7.59 (br s, 4H), 7.52-7.45 (m, 1H), 5.21 (s, 2H), 2.12 (s, 3H).

### Step 3: (4-(5-(5-Fluoropyridin-2-yl)-2-(pyridin-3-yl)-3H-imidazo[4,5-b]pyridin-3-yl)phenyl)methanol

To a solution of 4-(5-(5-fluoropyridin-2-yl)-2-(pyridin-3-yl)-3H-imidazo[4,5-b]pyridin-3-yl)benzyl acetate (700 mg, 1.59 mmol) in THF (5 mL) were added K₂CO₃ (661 mg, 4.78 mmol), MeOH (5 mL) and H₂O (5 mL). The mixture was stirred at 25° C. for 1 hr. The reaction mixture was diluted with H₂O (30 mL) and extracted with CH₂Cl₂ (20 mL×3). The combined organic layers were washed with brine (60 mL), dried over Na₂SO₄, filtered and concentrated under reduced pressure. (4-(5-(5-Fluoropyridin-2-yl)-2-(pyridin-3-yl)-3H-imidazo[4,5-b]pyridin-3-yl)phenyl)methanol (360 mg, crude) was obtained as a yellow solid. MS: m/z=398.1 [M+H]⁺.

### Step 4: 3-(4-(Chloromethyl)phenyl)-5-(5-fluoropyridin-2-yl)-2-(pyridin-3-yl)-3H-imidazo[4,5-b]pyridine

To a solution of (4-(5-(5-fluoropyridin-2-yl)-2-(pyridin-3-yl)-3H-imidazo[4,5-b]pyridin-3-yl)phenyl)methanol (360 mg, 508 μmol) in CH₂Cl₂ (5 mL) was added SOCl₂ (1 mL). The mixture was stirred at 40° C. for 1 hr. The mixture was concentrated under reduced pressure to give 3-(4-(chloromethyl)phenyl)-5-(5-fluoropyridin-2-yl)-2-(pyridin-3-yl)-3H-imidazo[4,5-b]pyridine (Intermediate 228, 410 mg, HCl salt, crude) as a yellow solid. MS: m/z=416.0 [M+H]⁺.

### Intermediate 229: 2-(3-(4-(Chloromethyl)phenyl)-5-(5-fluoropyridin-2-yl)-3H-imidazo[4,5-b]pyridin-2-yl)aniline

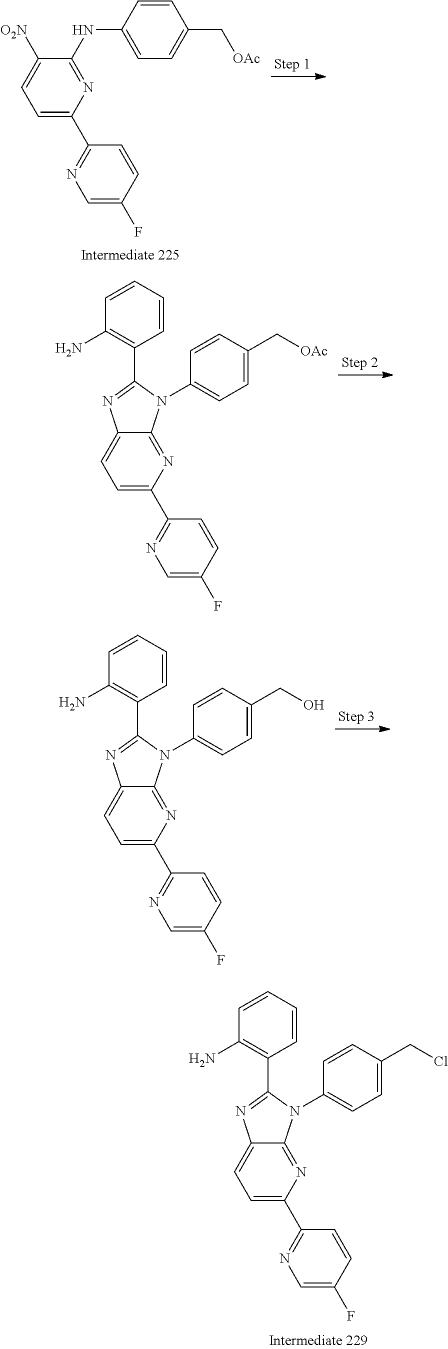 [SMILES: CC(=O)OCc1ccc(Nc2nc(-c3ccc(F)cn3)ccc2[N+](=O)[O-])cc1 ; CC(=O)OCc1ccc(-n2c(-c3ccccc3N)nc3ccc(-c4ccc(F)cn4)nc32)cc1 ; Nc1ccccc1-c1nc2ccc(-c3ccc(F)cn3)nc2n1-c1ccc(CO)cc1 ; Nc1ccccc1-c1nc2ccc(-c3ccc(F)cn3)nc2n1-c1ccc(CCl)cc1]

### Step 1: 4-(2-(2-Aminophenyl)-5-(5-fluoropyridin-2-yl)-3H-imidazo[4,5-b]pyridin-3-yl)benzyl acetate

A mixture of Intermediate 225 (1 g, 2.62 mmol), 2-aminobenzaldehyde (348 mg, 2.88 mmol), Na₂S₂O₄ (1.82 g, 10.5 mmol) in DMSO (5 mL) was degassed and purged with N₂ three times. The mixture was stirred at 100° C. for 16 hr under N₂ atmosphere. The reaction mixture was diluted with H₂O (30 mL) and extracted with EtOAc (20 mL×3). The combined organic layers were washed with brine (30 mL), dried over Na₂SO₄, filtered, and concentrated under reduced pressure. After purified by silica gel flash chromatography (Eluent of 0˜20% EtOAc in petroleum ether), 4-(2-(2-aminophenyl)-5-(5-fluoropyridin-2-yl)-3H-imidazo[4,5-b]pyridin-3-yl)benzyl acetate (150 mg, yield: 9%) was obtained as a yellow solid. MS: m/z=454.1 [M+H]+. H NMR (400 MHz, Dimethysulfoxide-d₅) 8.66 (d, J=2.8 Hz, 1H), 8.39 (d, J=8.4 Hz, 1H), 8.30-8.22 (m, 2H), 7.88-7.79 (m, 1H), 7.55-7.46 (m, 4H), 7.10-7.02 (m, 1H), 6.89-6.77 (m, 2H), 6.41-6.33 (m, 1H), 6.18 (br s, 2H), 5.18 (s, 2H), 2.12 (s, 3H).

### Step 2: (4-(2-(2-Aminophenyl)-5-(5-fluoropyridin-2-yl)-3H-imidazo[4,5-b]pyridin-3-yl)phenyl)methanol

To a solution of 4-(2-(2-aminophenyl)-5-(5-fluoropyridin-2-yl)-3H-imidazo[4,5-b]pyridin-3-yl)benzyl acetate (150 mg, 331 μmol) in MeOH (2 mL) and THF (2 mL) was added K₂CO₃ (137 mg, 992 μmol) in H₂O (1 mL). The mixture was stirred at 25° C. for 0.5 hr. The reaction mixture was diluted with H₂O (30 mL) and extracted with EtOAc (20 mL×3). The combined organic layers were washed with brine (30 mL), dried over Na₂SO₄, filtered and concentrated under reduced pressure. (4-(2-(2-Aminophenyl)-5-(5-fluoropyridin-2-yl)-3H-imidazo[4,5-b]pyridin-3-yl)phenyl)methanol (155 mg, crude) was obtained as a yellow solid. MS: m/z=412.1 [M+H]⁺

### Step 3: 2-(3-(4-(Chloromethyl)phenyl)-5-(5-fluoropyridin-2-yl)-3H-imidazo[4,5-b]pyridin-2-yl)aniline

To a solution of 4-(2-(2-aminophenyl)-5-(5-fluoropyridin-2-yl)-3H-imidazo[4,5-b]pyridin-3-yl)phenyl)methanol (155 mg, 377 μmol) in CH₂Cl₂ (2 mL) was added SOCl₂ (90 mg, 753 μmol). The mixture was stirred at 25° C. for 1 hr. The mixture was concentrated under reduced pressure to give 2-(3-(4-(chloromethyl)phenyl)-5-(5-fluoropyridin-2-yl)-3H-imidazo[4,5-b]pyridin-2-yl)aniline (Intermediate 229, 175 mg, HCl salt, crude) as a yellow solid, which was used in the next step without further purification. MS: m/z=430.0 [M+H]⁺.

### Intermediate 230: 3-(4-(Chloromethyl)phenyl)-5-(5-fluoropyridin-2-yl)-2-phenyl-3H-imidazo[4,5-b]pyridine

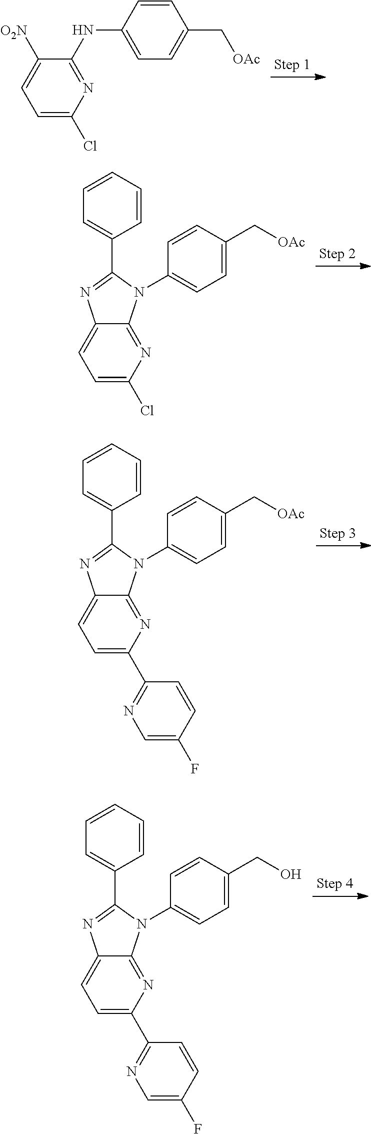

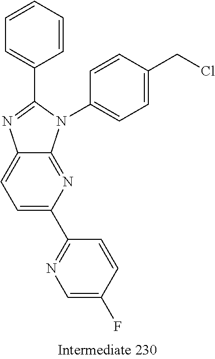 [SMILES: Fc1ccc(-c2ccc3nc(-c4ccccc4)n(-c4ccc(CCl)cc4)c3n2)nc1]

### Step 1; 4-(5-Chloro-2-phenyl-3H-imidazo[4,5-b]pyridin-3-yl)benzyl acetate

To a solution of 4-((6-chloro-3-nitropyridin-2-yl)amino)benzyl acetate (refer to Intermediate 178 for detail procedures, 1.4 g, 3.8 mmol) in DMSO (10 mL) were added Na₂S₂O₄ (2 g, 6.22 mmol, 87% purity) and benzaldehyde (792 mg, 7.46 mmol). The mixture was stirred at 100° C. for 16 hr. The reaction mixture was quenched with H₂O (50 mL) and extracted with EtOAc (50 mL×2). The combined organic layers were washed with brine (50 mL×2), dried over Na₂SO₄, filtered, and concentrated under reduced pressure. After purified by silica gel flash chromatography (Eluent of 0%˜43% EtOAc in petroleum ether), 4-(5-chloro-2-phenyl-3H-imidazo[4,5-b]pyridin-3-yl)benzyl acetate (440 mg, yield: 18%) was obtained as a light yellow solid. MS: m/z=378.2 [M+H]⁺. ¹H NMR (400 MHz, Chloroform-d) δ 8.09 (d, J=8.4 Hz, 1H), 7.58-7.54 (m, 2H), 7.48 (d, J=8.4 Hz, 2H), 7.41 (d, J=7.2 Hz, 1H), 7.37-7.32 (m, 5H), 5.18 (s, 2H), 2.15 (s, 3H).

### Step 2: 4-(5-(5-Fluoropyridin-2-yl)-2-phenyl-3H-imidazo[4,5-b]pyridin-3-yl)benzyl acetate

A mixture of 4-(5-chloro-2-phenyl-3H-imidazo[4,5-b]pyridin-3-yl)benzyl acetate (360 mg, 953 μmol), (5-fluoropyridin-2-yl)boronic acid (403 mg, 2.86 mmol), Pd(dppf)Cl₂ (69.7 mg, 95.3 μmol), Cs₂CO₃ (931 mg, 2.86 mmol) and CuBr (54.7 mg, 381 μmol) in 1,4-dioxane (7.5 mL) and H₂O (1.5 mL) was degassed and purged with N₂ three times. The mixture was stirred at 100° C. for 8 hr under N₂ atmosphere. The reaction mixture was quenched with H₂O (100 mL), diluted with CH₂Cl₂ (100 mL), and extracted with CH₂Cl₂ (100 mL×2). The combined organic layers were washed with brine (100 mL), dried over Na₂SO₄, filtered, and concentrated under reduced pressure. After purified by silica gel flash chromatography (Eluent of 0%˜35% EtOAc in petroleum ether), 4-(5-(5-fluoropyridin-2-yl)-2-phenyl-3H-imidazo[4,5-b]pyridin-3-yl)benzyl acetate (110 mg, yield: 24%) was obtained as a light yellow solid. MS: m/z=439.1 [M+H]+. ¹H NMR (400 MHz, Chloroform-d) δ 8.50 (d, J=2.8 Hz, 1H), 8.47 (d, J=8.4 Hz, 1H), 8.36 (dd, J=8.8, 4.8 Hz, 1H), 8.22 (d, J=8.4 Hz, 1H), 7.62 (d, J=7.6 Hz, 2H), 7.52-7.50 (m, 2H), 7.46-7.43 (m, 4H), 7.38 (d, J=8.0 Hz, 2H), 5.23 (s, 2H), 2.18 (s, 3H).

### Step 3: (4-(5-(5-Fluoropyridin-2-yl)-2-phenyl-3H-imidazo[4,5-b]pyridin-3-yl)phenyl)methanol

To a solution of 4-(5-(5-fluoropyridin-2-yl)-2-phenyl-3H-imidazo[4,5-b]pyridin-3-yl)benzyl acetate (110 mg, 251 μmol) in THF (2 mL) and MeOH (2 mL) were added K₂CO₃ (104 mg, 753 μmol) and H₂O (1 mL). The mixture was stirred at 25° C. for 1 hr. The reaction mixture was quenched with H₂O (50 mL) at 25° C. and extracted with EtOAc (50 mL×2). The combined organic layers were washed with brine (50 mL×2), dried over Na₂SO₄, filtered and concentrated under reduced pressure. (4-(5-(5-Fluoropyridin-2-yl)-2-phenyl-3H-imidazo[4,5-b]pyridin-3-yl)phenyl)methanol (99 mg, yield: 99%) was obtained as a light yellow solid. MS: m/z=397.1 [M+H]⁺.

### Step 4: 3-(4-(Chloromethyl)phenyl)-5-(5-fluoropyridin-2-yl)-2-phenyl-3H-imidazo[4,5-b]pyridine

To a solution of (4-(5-(5-fluoropyridin-2-yl)-2-phenyl-3H-imidazo[4,5-b]pyridin-3-yl)phenyl)methanol (99 mg, 250 μmol) in CH₂Cl₂ (2 mL) was added SOCl₂ (89.1 mg, 749 mol). The mixture was stirred at 40° C. for 0.5 hr. The reaction was concentrated under reduced pressure to give 3-(4-(chloromethyl)phenyl)-5-(5-fluoropyridin-2-yl)-2-phenyl-3H-imidazo[4,5-b]pyridine (Intermediate 230, 103 mg, HCl salt) as a light yellow solid. MS: m/z=415.1 [M+H]⁺.

### Intermediate 231: 2-Bromo-5-(methoxy-d₃)pyridine

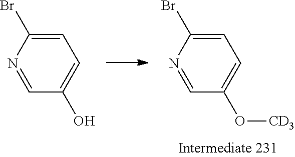 [SMILES: Oc1ccc(Br)nc1 ; [2H]C([2H])([2H])Oc1ccc(Br)nc1]

To a solution of 6-bromopyridin-3-ol (1 g, 5.8 mmol), CD₃OD (1.2 g, 34.5 mmol) and PPh₃ (1.7 g, 6.3 mmol) in 1,4-dioxane (10 mL) was added DIAD (1.4 mg, 6.9 mmol) at 0° C. The mixture was stirred at 25° C. for 4 hr. The mixture was quenched with H₂O (50 mL) and extracted with CH₂Cl₂ (20 mL×2). The combined organic layers were washed with brine (50 mL×3), dried over Na₂SO₄, filtered and concentrated. After purified by silica gel flash chromatography (Eluent of 0%˜15% EtOAc in petroleum ether), 2-bromo-5-(methoxy-d₃)pyridine (Intermediate 231, 1 g, yield: 84%) was obtained as a light yellow oil. MS: m/z=190.7, 192.7 [M+H]⁺. ¹H NMR (400 MHz, Dimethylsulfoxide-d₆) δ 8.12 (d, J=3.2 Hz, 1H), 7.55 (d, J=8.8 Hz, 1H), 7.39 (dd, J=8.8, 3.2 Hz, 1H).

### Intermediate 232: 3-(3-(4-(Chloromethyl)phenyl)-5-(5-(methoxy-d₃)pyridin-2-yl)-3H-imidazo[4,5-b]pyridin-2-yl)pyridin-2-amine

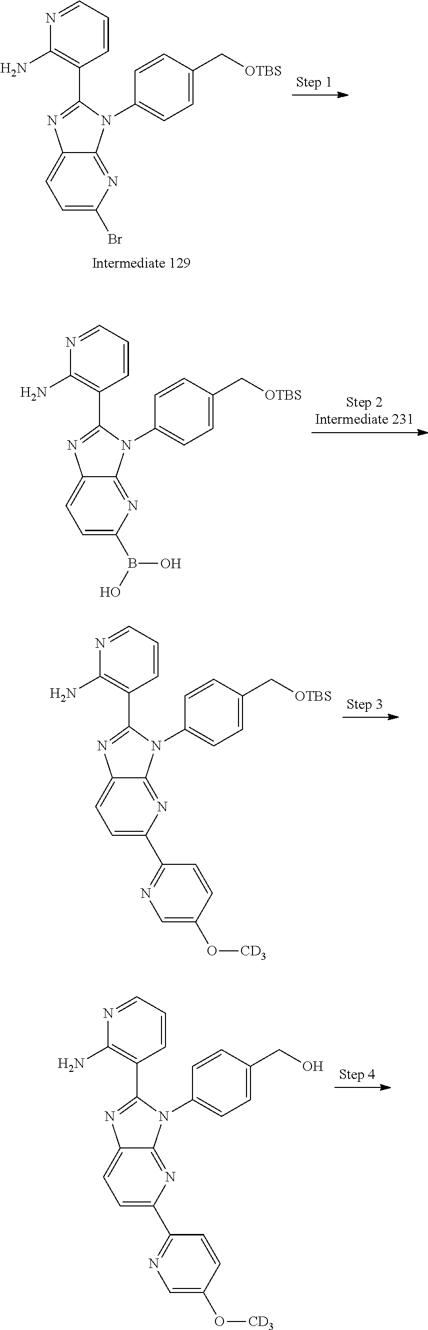

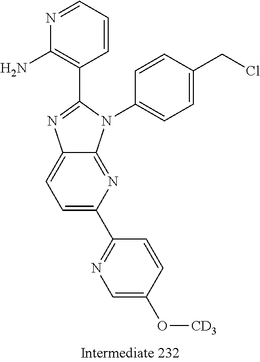 [SMILES: [2H]C([2H])([2H])Oc1ccc(-c2ccc3nc(-c4cccnc4N)n(-c4ccc(CCl)cc4)c3n2)nc1]

### Step 1: (2-(2-Aminopyridin-3-yl)-3-(4-(((tert-butyldimethylsilyl)oxy)methyl)phenyl)-3H-imidazo[4,5-b]pyridin-5-yl)boronic acid

A mixture of Intermediate 129 (1.5 g, 2.9 mmol), 4,4,4′,4′,5,5,5′,5′-octamethyl-2,2′-bi(1,3,2-dioxaborolane) (1.1 g, 4.4 mmol), KOAc (865 mg, 8.8 mmol) and Pd(dppf)Cl₂ (120 mg, 147 μmol) in 1,4-dioxane (15 mL) was degassed and purged with N₂ three times. The mixture was stirred at 110° C. for 2 hr under N₂ atmosphere. (2-(2-Aminopyridin-3-yl)-3-(4-(((tert-butyldimethylsilyl)oxy)methyl)phenyl)-3H-imidazo[4,5-b]pyridin-5-yl)boronic acid was obtained as a black liquid, which was used in the next step directly without work-up and purification. MS: m/z=476.2 [M+H]⁺.

### Step 2: 3-(3-(4-(((tert-Butyldimethylsilyl)oxy)methyl)phenyl)-5-(5-(methoxy-d₃)pyridin-2-yl)-3H-imidazo[4,5-b]pyridin-2-yl)pyridin-2-amine

A mixture of (2-(2-aminopyridin-3-yl)-3-(4-(((tert-butyldimethylsilyl)oxy)methyl)phenyl)-3H-imidazo[4,5-b]pyridin-5-yl)boronic acid (426 mg, 898 μmol), Intermediate 231 (206 mg, 1.0 mmol), Pd(dppf)Cl₂ (73 mg, 90 μmol) and Cs₂CO₃ (877 mg, 2.7 mmol) in 1,4-dioxane (5 mL) and H₂O (1 mL) was degassed and purged with N₂ three times. The mixture was stirred at 95° C. for 12 hr under N₂ atmosphere. The reaction mixture was quenched with H₂O (30 mL) and extracted with CH₂Cl₂ (20 mL×2). The combined organic layers were washed with brine (50 mL), dried over Na₂SO₄, filtered, and concentrated under reduced pressure. After purified by silica gel flash chromatography (Eluent of 0%˜25% EtOAc in petroleum ether), 3-(3-(4-(((tert-butyldimethylsilyl)oxy)methyl)phenyl)-5-(5-(methoxy-d₃)pyridin-2-yl)-3H-imidazo[4,5-b]pyridin-2-yl)pyridin-2-amine (240 mg, yield: 44% for two steps) was obtained as a black solid. MS: m/z=542.4 [M+H]⁺. D %: 3D %=95.3%. ¹H NMR (400 MHz, Dimethylsulfoxide-d₆) δ 8.39-8.34 (m, 2H), 8.26 (d, J=8.4 Hz, 1H), 8.12 (d, J=8.8 Hz, 1H), 8.00 (dd, J=4.4, 1.6 Hz, 1H), 7.51-7.48 (m, 4H), 7.47-7.45 (m, 1H), 7.19 (dd, J=7.6, 1.6 Hz, 1H), 7.01 (s, 2H), 6.39 (dd, J=7.6, 4.8 Hz, 1H), 4.84 (s, 2H), 0.93 (s, 9H), 0.12 (s, 6H).

### Step 3: (4-(2-(2-Aminopyridin-3-yl)-5-(5-(methoxy-d₃)pyridin-2-yl)-3H-imidazo[4,5-b]pyridin-3-yl)phenyl)methanol

To a solution of 3-(3-(4-(((tert-butyldimethylsilyl)oxy)methyl)phenyl)-5-(5-(methoxy-d₃)pyridin-2-yl)-3H-imidazo[4,5-b]pyridin-2-yl)pyridin-2-amine (200 mg, 369 μmol) in THF (3 mL) was added TBAF (3.7 mL, 3.7 mmol, 1 M in THF). The mixture was stirred at 25° C. for 2 hr. The reaction mixture was quenched with H₂O (5 mL) at 25° C. and extracted with CH₂Cl₂ (15 mL×2). The combined organic layers were washed with brine (30 mL×2), dried over Na₂SO₄, filtered and concentrated under reduced pressure. After triturated with EtOAc: petroleum ether=1:10 (11 mL) at 25° C. for 10 min, (4-(2-(2-aminopyridin-3-yl)-5-(5-(methoxy-d₃)pyridin-2-yl)-3H-imidazo[4,5-b]pyridin-3-yl)phenyl)methanol (155 mg, crude) was obtained as a brown solid. MS: m/z=428.0 [M+H]⁺. D %: 3D %=99.5%. ¹H NMR (400 MHz, Dimethylsulfoxide-d₆) δ 8.40-8.34 (m, 2H), 8.26 (d, J=8.4 Hz, 1H), 8.13 (d, J=9.2 Hz, 1H), 8.00 (dd, J=4.8, 1.6 Hz, 1H), 7.53-7.41 (m, 5H), 7.22 (dd, J=8.0, 1.6 Hz, 1H), 6.97 (s, 2H), 6.42 (dd, J=7.6, 4.8 Hz, 1H), 5.40-5.30 (m, 1H), 4.61 (d, J=5.2 Hz, 2H).

### Step 4: 3-(3-(4-(Chloromethyl)phenyl)-5-(5-(methoxy-d₃)pyridin-2-yl)-3H-imidazo[4,5-b]pyridin-2-yl)pyridin-2-amine

To a solution of (4-(2-(2-aminopyridin-3-yl)-5-(5-(methoxy-d₃)pyridin-2-yl)-3H-imidazo[4,5-b]pyridin-3-yl)phenyl)methanol (150 mg, 351 μmol) in CH₂Cl₂ (3 mL) was added SOCl₂ (209 mg, 1.7 mmol). The mixture was stirred at 40° C. for 1 hr. The reaction was concentrated under reduced pressure to give 3-(3-(4-(chloromethyl)phenyl)-5-(5-(methoxy-d₃)pyridin-2-yl)-3H-imidazo[4,5-b]pyridin-2-yl)pyridin-2-amine (Intermediate 232, 150 mg, HCl salt, crude) as a yellow solid. MS: m/z=446.0 [M+H]⁺. D %: 3D=99.7%.

### Intermediate 233: 3-(3-(4-(Chloromethyl)phenyl)-5-(5-(difluoromethoxy)pyridin-2-yl)-3H-imidazo[4,5-b]pyridin-2-yl)pyridin-2-amine

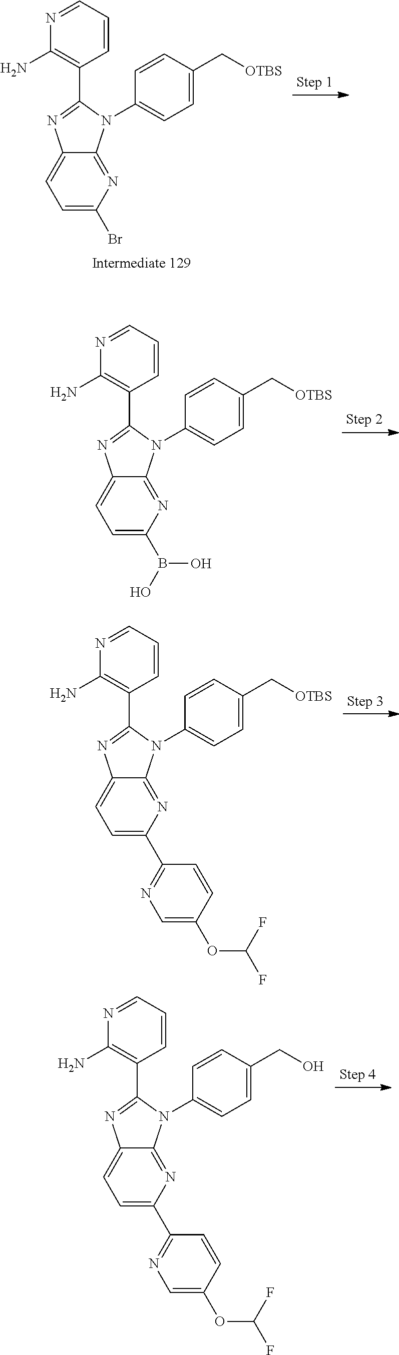

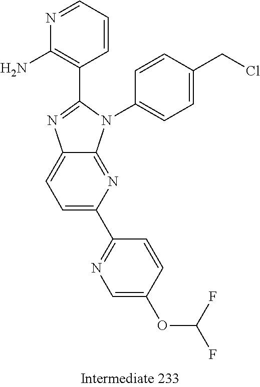 [SMILES: Nc1ncccc1-c1nc2ccc(-c3ccc(OC(F)F)cn3)nc2n1-c1ccc(CCl)cc1]

### Step 1: (2-(2-Aminopyridin-3-yl)-3-(4-(((tert-butyldimethylsilyl)oxy)methyl)phenyl)-3H-imidazo[4,5-b]pyridin-5-yl)boronic acid

A mixture of Intermediate 129 (1 g, 1.96 mmol), 4,4,4′,4′,5, 5,5′,5′-octamethyl-2,2′-bi(1,3,2-dioxaborolane) (995 mg, 3.92 mmol), KOAc (577 mg, 5.88 mmol) and Pd(dppf)Cl₂ (143 mg, 196 mmol) in 1,4-dioxane (5 mL) was degassed and purged with N₂ three times. The mixture was stirred at 90° C. for 16 hr under N₂ atmosphere. (2-(2-Aminopyridin-3-yl)-3-(4-(((tert-butyldimethylsilyl)oxy)methyl)phenyl)-3H-imidazo[4,5-b]pyridin-5-yl)boronic acid was obtained as a black liquid, which was used in the next step without work-up and purification. MS: m/z=476.2 [M+H]⁺.

### Step 2: 3-(3-(4-(((tert-Butyldimethylsilyl)oxy)methyl)phenyl)-5-(5-(difluoromethoxy)pyridin-2-yl)-3H-imidazo[4,5-h]pyridin-2-yl)pyridin-2-amine

A mixture of (2-(2-aminopyridin-3-yl)-3-(4-(((tert-butyldimethylsilyl)oxy)methyl)phenyl)-3H-imidazo[4,5-b]pyridin-5-yl)boronic acid (470 mg, 989 μmol), 2-bromo-5-(difluoromethoxy)pyridine (266 mg, 1.19 mmol), Pd(dppf)Cl₂ (72.3 mg, 98.9 μmol) and Cs₂CO₃ (966 mg, 2.97 mmol) in 1,4-dioxane (4 mL) and H₂O (1 mL) was degassed and purged with N₂ three times. The mixture was stirred at 90° C. for 16 hr under N₂ atmosphere. The reaction mixture was poured into H₂O (15 mL) and extracted with EtOAc (15 mL×3). The combined organic phases were dried over anhydrous Na₂SO₄, filtered and concentrated under reduced pressure. After purified by silica gel flash chromatography (Eluent of 0%˜56% EtOAc in petroleum ether), 3-(3-(4-(((tert-butyldimethylsilyl)oxy)methyl)phenyl)-5-(5-(difluoromethoxy)pyridin-2-yl)-3H-imidazo[4,5-b]pyridin-2-yl)pyridin-2-amine (210 mg, yield: 37% for two steps) was obtained as a brown solid. MS: m/z=575.2 [M+H]⁺. ¹H NMR (400 MHz, Dimethysulfoxide-d₆) δ 8.57 (d, J=8.4 Hz, 1H), 8.40 (d, J=8.4 Hz, 1H), 8.31 (d, J=8.4 Hz, 1H), 8.21 (d, J=8.8 Hz, 1H), 8.00 (dd, J=4.8, 2.0 Hz, 1H), 7.76 (dd, J=8.4, 2.4 Hz, 1H), 7.54-7.45 (m, 4H), 7.41-7.27 (m, 1H), 7.22-7.16 (m, 1H), 7.00 (s, 2H), 6.34 (dd, J=7.6, 4.8 Hz, 1H), 4.82 (s, 2H), 0.93 (s, 9H), 0.12 (s, 6H). ¹⁹F NMR (400 MHz, Dimethysulfoxide-d₆) δ-82.487.

### Step 3: (4-(2-(2-Aminopyridin-3-yl)-5-(5-(difluoromethoxy)pyridin-2-yl)-3H-imidazo[4,5-b]pyridin-3-yl)phenyl)methanol

To a solution of 3-(3-(4-(((tert-butyldimethylsilyl)oxy)methyl)phenyl)-5-(5-(difluoromethoxy)pyridin-2-yl)-3H-imidazo[4,5-b]pyridin-2-yl)pyridin-2-amine (210 mg, 365 μmol) in THF (3 mL) was added TBAF (1 mL, 1 M in THF). The mixture was stirred at 25° C. for 0.3 hr. The reaction mixture was poured into H₂O (15 mL) and extracted with EtOAc (15 mL×3). The combined organic layers were dried over anhydrous Na₂SO₄, filtered, and concentrated under reduced pressure. (4-(2-(2-Aminopyridin-3-yl)-5-(5-(difluoromethoxy)pyridin-2-yl)-3H-imidazo[4,5-b]pyridin-3-yl)phenyl)methanol (101 mg, crude) was obtained as a brown solid. MS: m/z=461.1 [M+H]⁺.

### Step 4: 3-(3-(4-(Chloromethyl)phenyl)-5-(5-(difluoromethoxy)pyridin-2-yl)-3H-imidazo[4,5-b]pyridin-2-yl)pyridin-2-amine

To a solution of (4-(2-(2-aminopyridin-3-yl)-5-(5-(difluoromethoxy)pyridin-2-yl)-3H-imidazo[4,5-b]pyridin-3-yl)phenyl)methanol (101 mg, 219 μmol) in CH₂Cl₂ (4 mL) was added SOCl₂ (1 mL). The mixture was stirred at 25° C. for 1 hr. The reaction was concentrated under reduced pressure to give 3-(3-(4-(chloromethyl)phenyl)-5-(5-(difluoromethoxy)pyridin-2-yl)-3H-imidazo[4,5-b]pyridin-2-yl)pyridin-2-amine (Intermediate 233, 115 mg, HCl salt) as a brown solid. MS: m/z=479.1 [M+H]⁺.

### Intermediate 234: 2-Bromo-5-(fluoromethoxy)pyridine

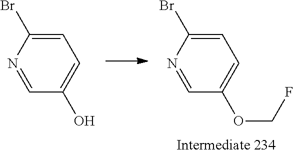 [SMILES: Oc1ccc(Br)nc1 ; FCOc1ccc(Br)nc1]

To a solution of 6-bromopyridin-3-ol (1 g, 5.8 mmol) and K₂CO₃ (2.4 g, 17.2 mmol) in DMF (10 mL) was added bromofluoromethane (974 mg, 8.6 mmol) at 0° C. The mixture was stirred at 0° C. for 2 hr. The mixture was quenched with H₂O (50 mL) and extracted with CH₂Cl₂ (20 mL×2). The combined organic layers were washed with brine (50 mL×3), dried over Na₂SO₄, filtered and concentrated. After purified by silica gel flash chromatography (Eluent of 0%˜15% EtOAc in petroleum ether), 2-bromo-5-(fluoromethoxy)pyridine (Intermediate 234, 1 g, yield: 84%) was obtained as a light yellow oil. MS: m/z=205.7, 207.7 [M+H]⁺. ¹H NMR (400 MHz, Dimethylsulfoxide-d₆) δ 8.27 (d, J=3.0 Hz, 1H), 7.66 (d, J=8.8 Hz, 1H), 7.61-7.55 (m, 1H), 5.93 (d, J=53.6 Hz, 2H). ¹⁹F NMR (400 MHz, Dimethylsulfoxide-d₆) 6-152.277.

### Intermediate 235: 3-(3-(4-(Chloromethyl)phenyl)-5-(5-(fluoromethoxy)pyridin-2-yl)-3H-imidazo[4,5-b]pyridin-2-yl)pyridin-2-amine

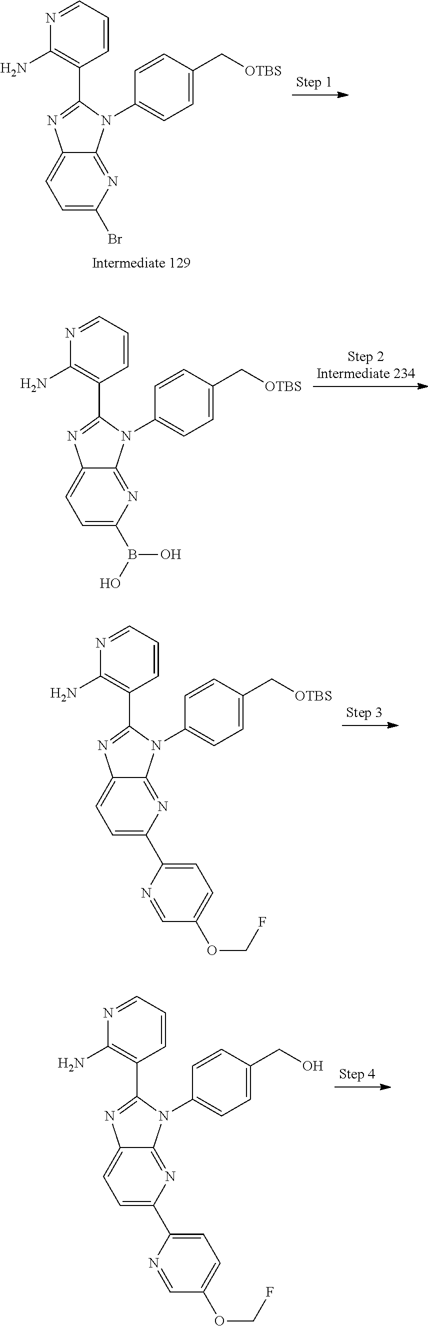

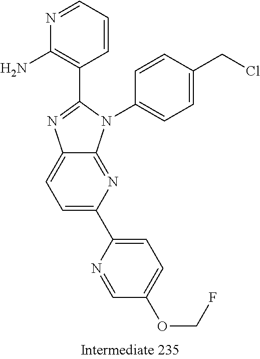 [SMILES: Nc1ncccc1-c1nc2ccc(-c3ccc(OCF)cn3)nc2n1-c1ccc(CCl)cc1]

### Step 1: (2-(2-Aminopyridin-3-yl)-3-(4-(((tert-butyldimethylsilyl)oxy)methyl)phenyl)-3H-imidazo[4,5-b]pyridin-5-yl)boronic acid

A mixture of Intermediate 129 (1.5 g, 2.9 mmol), 4,4,4′,4′,5,5,5′,5′-octamethyl-2,2′-bi(1,3,2-dioxaborolane) (1.1 g, 4.4 mmol), KOAc (865 mg, 8.8 mmol) and Pd(dppf)Cl₂ (120 mg, 147 mol) in 1,4-dioxane (15 mL) was degassed and purged with N₂ three times. The mixture was stirred at 110° C. for 2 hr under N₂ atmosphere. (2-(2-Aminopyridin-3-yl)-3-(4-(((tert-butyldimethylsilyl)oxy)methyl)phenyl)-3H-imidazo[4,5-b]pyridin-5-yl)boronic acid was obtained as a black liquid, which was used in the next step without work-up and purification. MS: m/z=476.2 [M+H]⁺.

### Step 2: 3-(3-(4-(((tert-Butyldimethylsilyl)oxy)methyl)phenyl)-5-(5-(fluoromethoxy)pyridin-2-yl)-3H-imidazo[4,5-b]pyridin-2-yl)pyridin-2-amine

A mixture of (2-(2-Aminopyridin-3-yl)-3-(4-(((tert-butyldimethylsilyl)oxy)methyl)phenyl)-3H-imidazo[4,5-b]pyridin-5-yl)boronic acid (426 mg, 897 μmol), Intermediate 234 (221 mg, 1.1 mmol), Pd(dppf)Cl₂ (73 mg, 90 μmol) and Cs₂CO₃ (876 mg, 2.7 mmol) in 1,4-dioxane (5 mL) was degassed and purged with N₂ three times. The mixture was stirred at 95° C. for 12 hr under N₂ atmosphere. The reaction mixture was quenched with H₂O (30 mL) and extracted with CH₂Cl₂ (20 mL×2). The combined organic layers were washed with brine (50 mL), dried over Na₂SO₄, filtered, and concentrated under reduced pressure. After purified by silica gel flash chromatography (Eluent of 0%˜25% EtOAc in petroleum ether), 3-(3-(4-(((tert-butyldimethylsilyl)oxy)methyl)phenyl)-5-(5-(fluoromethoxy)pyridin-2-yl)-3H-imidazo[4,5-b]pyridin-2-yl)pyridin-2-amine (260 mg, yield: 46% for two steps) was obtained as a brown solid. MS: m/z=557.3 [M+H]⁺. ¹H NMR (400 MHz, Dimethylsulfoxide-d₆) δ 8.51 (d, J=2.8 Hz, 1H), 8.39 (d, J=8.4 Hz, 1H), 8.29 (d, J=8.4 Hz, 1H), 8.17 (d, J=8.8 Hz, 1H), 8.00 (dd, J=4.8, 1.6 Hz, 1H), 7.67 (dd, J=8.8, 2.8 Hz, 1H), 7.51-7.47 (m, 4H), 7.20 (dd, J=7.6, 2.0 Hz, 1H), 7.00 (s, 2H), 6.39 (dd, J=7.6, 4.8 Hz, 1H), 5.96 (d, J=53.6 Hz, 2H), 4.84 (s, 2H), 0.93 (s, 9H), 0.12 (s, 6H). ¹⁹F NMR (400 MHz, Dimethylsulfoxide-d₆) δ −151.294.

### Step 3: (4-(2-(2-Aminopyridin-3-yl)-5-(5-(fluoromethoxy)pyridin-2-yl)-3H-imidazo[4,5-b]pyridin-3-yl)phenyl)methanol

To a solution of 3-(3-(4-(((tert-butyldimethylsilyl)oxy)methyl)phenyl)-5-(5-(fluoromethoxy)pyridin-2-yl)-3H-imidazo[4,5-b]pyridin-2-yl)pyridin-2-amine (200 mg, 359 μmol) in THF (3 mL) was added TBAF (3.6 mL, 3.6 mmol, 1 M in THF). The mixture was stirred at 25° C. for 2 hr. The reaction mixture was quenched with H₂O (5 mL) at 25° C. and extracted with CH₂Cl₂ (15 mL×2). The combined organic layers were washed with brine (30 mL×2), dried over Na₂SO₄, filtered and concentrated under reduced pressure. After triturated with EtOAc: petroleum ether=1:10 (11 mL) at 25° C. for 10 min, (4-(2-(2-aminopyridin-3-yl)-5-(5-(fluoromethoxy)pyridin-2-yl)-3H-imidazo[4,5-b]pyridin-3-yl)phenyl)methanol (155 mg, crude) was obtained as a brown solid. MS: m/z=442.9 [M+H]+. ¹H NMR (400 MHz, Dimethylsulfoxide-d₆) δ 8.51 (d, J=2.8 Hz, 1H), 8.39 (d, J=8.4 Hz, 1H), 8.29 (d, J=8.4 Hz, 1H), 8.18 (d, J=8.8 Hz, 1H), 8.01 (dd, J=4.8, 1.6 Hz, 1H), 7.69 (dd, J=8.8, 2.8 Hz, 1H), 7.52-7.45 (m, 4H), 7.23 (dd, J=7.6, 1.6 Hz, 1H), 6.96 (s, 2H), 6.42 (dd, 0.1=7.6, 4.8 Hz, 1H), 5.96 (d, J=53.6 Hz, 2H), 5.43-5.33 (m, 1H), 4.61 (s, 2H). ¹⁹F NMR (400 MHz, Dimethylsulfoxide-d₆) δ −151.294.

### Step 4: 3-(3-(4-(Chloromethyl)phenyl)-5-(5-(fluoromethoxy)pyridin-2-yl)-3H-imidazo[4,5-b]pyridin-2-yl)pyridin-2-amine

To a solution of (4-(2-(2-aminopyridin-3-yl)-5-(5-(fluoromethoxy)pyridin-2-yl)-3H-imidazo[4,5-b]pyridin-3-yl)phenyl)methanol (150 mg, 339 μmol) in CH₂Cl₂ (5 mL) was added SOCl₂ (209 mg, 1.7 mmol). The mixture was stirred at 40° C. for 1 hr. The reaction was concentrated under reduced pressure to give 3-(3-(4-(chloromethyl)phenyl)-5-(5-(fluoromethoxy)pyridin-2-yl)-3H-imidazo[4,5-b]pyridin-2-yl)pyridin-2-amine (Intermediate 235, 150 mg, HCl salt, crude) as a yellow solid. MS: m/z=460.9 [M+H]⁺.

### Intermediate 236: 3-(3-(4-(Chloromethyl)phenyl)-5-(5-methoxypyrimidin-2-yl)-3H-imidazo[4,5-b]pyridin-2-yl)pyridin-2-amine

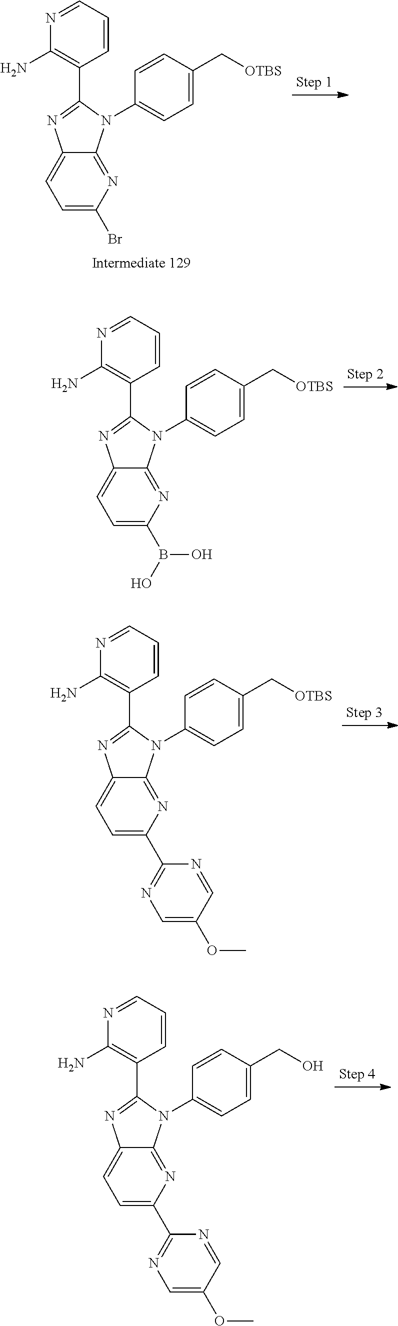

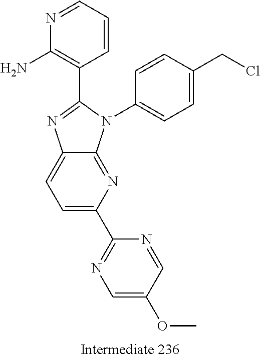 [SMILES: COc1cnc(-c2ccc3nc(-c4cccnc4N)n(-c4ccc(CCl)cc4)c3n2)nc1]

### Step 1; (2-(2-Aminopyridin-3-yl)-3-(4-(((tert-butyldimethylsilyl)oxy)methyl)phenyl)-3H-imidazo[4,5-b]pyridin-5-yl)boronic acid

A mixture of Intermediate 129 (500 mg, 979 μmol), 4,4,4′,4′,5,5,5′,5′-octamethyl-2,2′-bi(1,3,2-dioxaborolane) (497 mg, 1.96 mmol), KOAc (288 mg, 2.94 mmol) and Pd(dppf)Cl₂ (71.7 mg, 97.9 μmol) in 1,4-dioxane (2 mL) was degassed and purged with N₂ three times. The mixture was stirred at 80° C. for 16 hr under N₂ atmosphere. (2-(2-Aminopyridin-3-yl)-3-(4-(((tert-butyldimethylsilyl)oxy)methyl)phenyl)-3H-imidazo[4,5-b]pyridin-5-yl)boronic acid was obtained as a black liquid, which was used in the next step without work-up and purification. MS: m/z=476.2 [M+H]⁺.

### Step 2: 3-(3-(4-(((tert-Butyldimethylsilyl)oxy)methyl)phenyl)-5-(5-methoxypyrimidin-2-yl)-3H-imidazo[4,5-b]pyridin-2-yl)pyridin-2-amine

A mixture of (2-(2-aminopyridin-3-yl)-3-(4-(((tert-butyldimethylsilyl)oxy)methyl)phenyl)-3H-imidazo[4,5-b]pyridin-5-yl)boronic acid (465 mg, 978 μmol), 2-bromo-5-methoxypyrimidine (185 mg, 978 μmol), Pd(dppf)Cl₂ (71.6 mg, 97.8 μmol) and Cs₂CO₃ (956 mg, 2.93 mmol) in 1,4-dioxane (5 mL) and H₂O (1 mL) was degassed and purged with N₂ three times. The mixture was stirred at 90° C. for 2 hr under N₂ atmosphere. The reaction mixture was quenched with H₂O (5 mL), diluted with CH₂Cl₂ (10 mL), and extracted with CH₂Cl₂ (15 mL×2). The combined organic layers were washed with brine (50 mL), dried over Na₂SO₄, filtered and concentrated under reduced pressure. After purified by silica gel flash chromatography (Eluent of 30%˜60% EtOAc in petroleum ether), 3-(3-(4-(((tert-butyldimethylsilyl)oxy)methyl)phenyl)-5-(5-methoxypyrimidin-2-yl)-3H-imidazo[4,5-b]pyridin-2-yl)pyridin-2-amine (280 mg, yield: 46% for two steps) was obtained as a black solid. MS: m/z=540.2 [M+H]⁺. ¹H NMR (400 MHz, Chloroform-d) 68.53 (s, 2H), 8.48 (d, J=8.4 Hz, 1H), 8.19 (d, J=8.4 Hz, 1H), 8.06 (dd, J=4.8, 1.6 Hz, 1H), 7.51-7.41 (m, 4H), 7.08 (dd, J=7.6, 1.6 Hz, 1H), 6.62 (br s, 2H), 6.36 (dd, J=7.6, 4.8 Hz, 1H), 4.83 (s, 2H), 3.96 (s, 3H), 0.97 (s, 9H), 0.14 (s, 6H).

### Step 3: (4-(2-(2-Aminopyridin-3-yl)-5-(5-methoxypyrimidin-2-yl)-3H-imidazo[4,5-b]pyridin-3-yl)phenyl)methanol

To a solution of 3-(3-(4-(((tert-butyldimethylsilyl)oxy)methyl)phenyl)-5-(5-methoxypyrimidin-2-yl)-3H-imidazo[4,5-b]pyridin-2-yl)pyridin-2-amine (260 mg, 481.75 mol) in THF (5 mL) was added TBAF (378 mg, 1.45 mmol, 1 M in THF). The mixture was stirred at 25° C. for 1 hr. The reaction mixture was quenched with H₂O (5 mL) at 25° C. and extracted with CH₂Cl₂ (15 mL×2). The combined organic layers were washed with brine (30 mL×2), dried over Na₂SO₄, filtered and concentrated under reduced pressure. After triturated with EtOAc: petroleum ether=1:10 (11 mL) at 25° C. for 10 min, (4-(2-(2-aminopyridin-3-yl)-5-(5-methoxypyrimidin-2-yl)-3H-imidazo[4,5-b]pyridin-3-yl)phenyl)methanol (200 mg, yield: 92%) was obtained as a black solid. MS: m/z=426.2 [M+H]⁺. ¹H NMR (400 MHz, Dimethylsulfoxide-d₆) δ 8.66 (s, 2H), 8.39 (d, J=8.4 Hz, 1H), 8.29 (d, J=8.4 Hz, 1H), 7.99 (d, J=3.2 Hz, 1H), 7.52-7.47 (m, 2H), 7.45-7.40 (m, 2H), 7.22 (d, J=6.4 Hz, 1H), 6.98 (br s, 2H), 6.41 (dd, J=7.6, 4.8 Hz, 1H), 5.40 (t, J=5.6 Hz, 1H), 4.60 (d, J=5.6 Hz, 2H), 3.96 (s, 3H).

### Step 4: 3-(3-(4-(Chloromethyl)phenyl)-5-(5-methoxypyrimidin-2-yl)-3H-imidazo[4,5-b]pyridin-2-yl)pyridin-2-amine

To a solution of (4-(2-(2-aminopyridin-3-yl)-5-(5-methoxypyrimidin-2-yl)-3H-imidazo[4,5-b]pyridin-3-yl)phenyl)methanol (200 mg, 470 μmol) in CH₂Cl₂ (3 mL) was added SOCl₂ (280 mg, 2.35 mmol). The mixture was stirred at 25° C. for 0.5 hr. The reaction was concentrated under reduced pressure to give 3-(3-(4-(chloromethyl)phenyl)-5-(5-methoxypyrimidin-2-yl)-3H-imidazo[4,5-b]pyridin-2-yl)pyridin-2-amine (Intermediate 236, 226 mg, HCl salt) as a purple solid. MS: m/z=444.1 [M+H]⁺. ¹H NMR (400 MHz, Methanol-d₄) δ8.96 (s, 2H), 8.75 (d, 1=8.4 Hz, 1H), 8.54 (d, J=8.4 Hz, 1H), 8.05 (dd, J=6.0, 1.2 Hz, 1H), 7.85 (dd, J=7.6, 1.2 Hz, 1H), 7.74-7.70 (m, 2H), 7.64-7.60 (m, 2H), 6.87 (dd, J=7.2, 6.4 Hz, 1H), 4.80 (s, 2H), 4.16 (s, 3H).

### Intermediate 237: 3-(3-(4-(Chloromethyl)phenyl)-5-(5-methylpyrazin-2-yl)-3H-imidazo[4,5-b]pyridin-2-yl)pyridin-2-amine

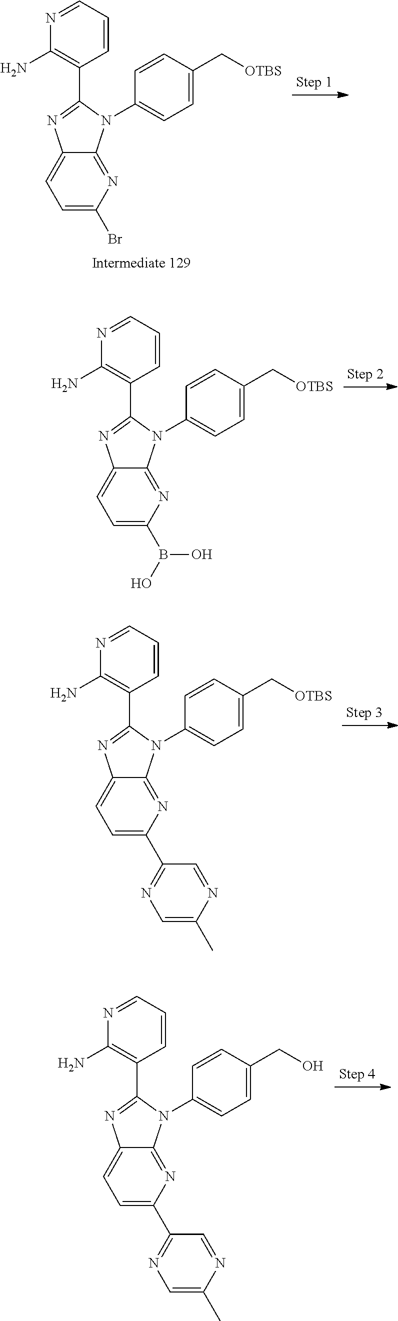

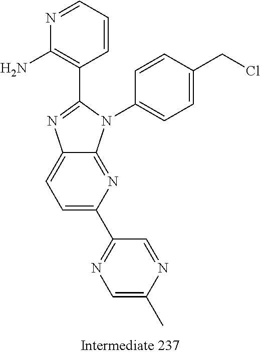 [SMILES: Cc1cnc(-c2ccc3nc(-c4cccnc4N)n(-c4ccc(CCl)cc4)c3n2)cn1]

### Step 1: (2-(2-Aminopyridin-3-yl)-3-(4-(((tert-butyldimethylsilyl)oxy)methyl)phenyl)-3H-imidazo[4,5-b]pyridin-5-yl)boronic acid

A mixture of Intermediate 129 (1 g, 1.96 mmol), 4,4,4′,4′,5,5,5′,5′-octamethyl-2,2′-bi(1,3,2-dioxaborolane) (746 mg, 2.94 mmol), KOAc (577 mg, 5.88 mmol) and Pd(dppf)Cl₂ (80 mg, 98 μmol) in 1,4-dioxane (10 mL) was degassed and purged with N₂ three times. The mixture was stirred at 80° C. for 12 hr under N₂ atmosphere. (2-(2-Aminopyridin-3-yl)-3-(4-(((tert-butyldimethylsilyl)oxy)methyl)phenyl)-3H-imidazo[4,5-h]pyridin-5-yl)boronic acid was obtained as a black liquid, which was used in next step without work-up and purification. MS: m/z=476.2 [M+H]⁺.

### Step 2: 3-(3-(4-(((tert-Butyldimethylsilyl)oxy)methyl)phenyl)-5-(5-methylpyrazin-2-yl)-3H-imidazo[4,5-b]pyridin-2-yl)pyridin-2-amine

A mixture of (2-(2-aminopyridin-3-yl)-3-(4-(((tert-butyldimethylsilyl)oxy)methyl)phenyl)-3H-imidazo[4,5-b]pyridin-5-yl)boronic acid (500 mg, 1.0 mmol), 2-bromo-5-methylpyrazine (218 mg, 1.3 mmol), Pd(dppf)Cl₂ (86 mg, 105 μmol) and Cs₂CO₃ (1.0 g, 3.2 mmol) in 1,4-dioxane (5 mL) and H₂O (1 mL) was degassed and purged with N₂ three times. The mixture was stirred at 90° C. for 2 hr under N₂ atmosphere. The reaction mixture was diluted with H₂O (15 mL) and extracted with EtOAc (15 mL×3). The combined organic layers were washed with brine (30 mL), dried over Na₂SO₄, filtered and concentrated under reduced pressure. After purified by silica gel flash chromatography (Eluent of 0%˜48% EtOAc in petroleum ether), 3-(3-(4-(((tert-butyldimethylsilyl)oxy)methyl)phenyl)-5-(5-methylpyrazin-2-yl)-3H-imidazo[4,5-b]pyridin-2-yl)pyridin-2-amine (240 mg, yield: 55% for two steps) was obtained as a black solid. MS: m/z=524.2 [M+H]⁺. ¹H NMR (400 MHz, Dimethylsulfoxide-d₆) δ 9.19 (s, 1H), 8.61 (s, 1H), 8.35 (d, J=2.4 Hz, 2H), 8.02 (dd, J=4.8, 1.6 Hz, 1H), 7.50-7.48 (m, 4H), 7.23 (dd, J=8.0, 2.0 Hz, 1H), 7.01 (s, 2H), 6.41 (dd, J=7.6, 4.8 Hz, 1H), 4.83 (s, 2H), 0.93-0.91 (m, 12H), 0.11 (s, 6H)

### Step 3: 4-(2-(2-Aminopyridin-3-yl)-5-(5-methylpyrazin-2-yl)-3H-imidazo[4,5-b]pyridin-3-yl)phenyl)methanol

A mixture of 3-(3-(4-(((tert-butyldimethylsilyl)oxy)methyl)phenyl)-5-(5-methylpyrazin-2-yl)-3H-imidazo[4,5-b]pyridin-2-yl)pyridin-2-amine (240 mg, 458 μmol) in THF (20 mL) was added TBAF (1 M, 917 μL). The mixture was stirred at 25° C. for 1 hr. The reaction mixture was diluted with H₂O (10 mL) and extracted with EtOAc (10 mL×3). The combined organic layers were washed with brine (30 mL), dried over Na₂SO₄, filtered and concentrated under reduced pressure. (4-(2-(2-Aminopyridin-3-yl)-5-(5-methylpyrazin-2-yl)-3H-imidazo[4,5-b]pyridin-3-yl)phenyl)methanol (171 mg, yield: 91%) was obtained as a yellow solid. MS: m/z=410.2 [M+H]⁺.

### Step 4: 3-(3-(4-(Chloromethyl)phenyl)-5-(5-methylpyrazin-2-yl)-3H-imidazo[4,5-b]pyridin-2-yl)pyridin-2-amine

To a solution of (4-(2-(2-Aminopyridin-3-yl)-5-(5-methylpyrazin-2-yl)-3H-imidazo[4,5-b]pyridin-3-yl)phenyl)methanol (171 mg, 417 μmol) in CH₂Cl₂ (10 mL) was added SOCl₂ (163 mg, 1.37 mmol). The mixture was stirred at 40° C. for 1 hr. The mixture was concentrated under reduced pressure to give 3-(3-(4-(chloromethyl)phenyl)-5-(5-methylpyrazin-2-yl)-3H-imidazo[4,5-b]pyridin-2-yl)pyridin-2-amine (Intermediate 237, 158 mg, HCl salt, yield: 79%) as a yellow solid. MS: m/z=428.1 [M+H]⁺.

### Intermediate 238: 3-(3-(4-(Chloromethyl)phenyl)-5-(5-methoxypyrazin-2-yl)-3H-imidazo[4,5-b]pyridin-2-yl)pyridin-2-amine

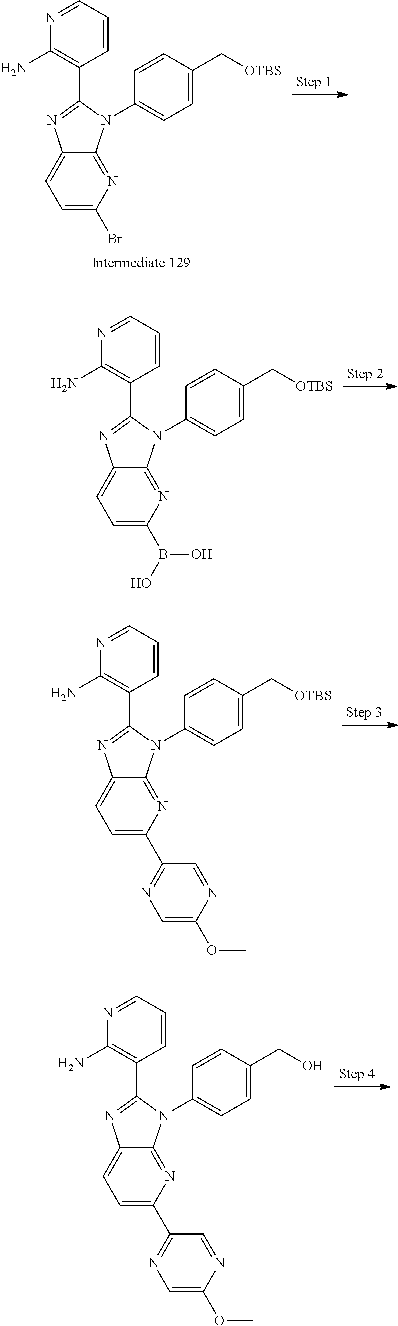

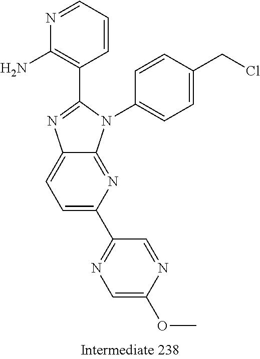 [SMILES: COc1cnc(-c2ccc3nc(-c4cccnc4N)n(-c4ccc(CCl)cc4)c3n2)cn1]

### Step 1: (2-(2-Aminopyridin-3-yl)-3-(4-(((tert-butyldimethylsilyl)oxy)methyl)phenyl)-3H-imidazo[4,5-b]pyridin-5-yl)boronic acid

A mixture of Intermediate 129 (1 g, 1.96 mmol), 4,4,4′,4′,5,5,5′,5′-octamethyl-2,2′-bi(1,3,2-dioxaborolane) (746 mg, 2.94 mmol), KOAc (577 mg, 5.88 mmol) and Pd(dppf)Cl₂ (80 mg, 98 μmol) in 1,4-dioxane (10 mL) was degassed and purged with N₂ three times. The mixture was stirred at 80° C. for 12 hr under N₂ atmosphere. (2-(2-Aminopyridin-3-yl)-3-(4-(((tert-butyldimethylsilyl)oxy)methyl)phenyl)-3H-imidazo[4,5-b]pyridin-5-yl)boronic acid was obtained as a black liquid, which was used in the next step without work-up and purification. MS: m/z=476.2 [M+H]⁺.

### Step 2: 3-(3-(4-(((tert-Butyldimethylsilyl)oxy)methyl)phenyl)-5-(5-methoxypyrazin-2-yl)-3H-imidazo[4,5-b]pyridin-2-yl)pyridin-2-amine

A mixture of (2-(2-aminopyridin-3-yl)-3-(4-(((tert-butyldimethylsilyl)oxy)methyl)phenyl)-3H-imidazo[4,5-b]pyridin-5-yl)boronic acid (500 mg, 1.0 mmol), 2-bromo-5-methoxypyrazine (218 mg, 1.3 mmol), Pd(dppf)Cl₂ (86 mg, 105 μmol) and Cs₂CO₃ (1.0 g, 3.2 mmol) in 1,4-dioxane (5 mL) and H₂O (1 mL) was degassed and purged with N₂ three times. The mixture was stirred at 90° C. for 2 hr under N₂ atmosphere. The reaction mixture was diluted with H₂O (15 mL) and extracted with EtOAc (15 mL×3). The combined organic layers were washed with brine (30 mL), dried over Na₂SO₄, filtered and concentrated under reduced pressure. After purified by silica gel flash chromatography (Eluent of 0%˜45% EtOAc in petroleum ether), 3-(3-(4-(((tert-butyldimethylsilyl)oxy)methyl)phenyl)-5-(5-methoxypyrazin-2-yl)-3H-imidazo[4,5-b]pyridin-2-yl)pyridin-2-amine (280 mg, yield: 70% for two steps) was obtained as a black solid. MS: m/z=540.2 [M+H]⁺. ¹H NMR (400 MHz, Dimethylsulfoxide-d₆) δ 8.90 (s, 1H), 8.39 (s, 1H), 8.31 (d, J=8.4 Hz, 1H), 8.25 (d, J=8.0 Hz, 1H), 8.01 (dd, J=4.8, 2.0 Hz, 1H), 7.50 (d, J=2.0 Hz, 4H), 7.23 (dd, J=7.2, 1.6 Hz, 1H), 7.02 (s, 2H), 6.40 (dd, J=7.6, 4.8 Hz, 1H), 4.83 (s, 2H), 0.93-0.92 (m, 12H), 0.12 (s, 6H)

### Step 3: (4-(2-(2-Aminopyridin-3-yl)-5-(5-methoxypyrazin-2-yl)-3H-imidazo[4,5-b]pyridin-3-yl)phenyl)methanol

To a mixture of 3-(3-(4-(((tert-butyldimethylsilyl)oxy)methyl)phenyl)-5-(5-methoxypyrazin-2-yl)-3H-imidazo[4,5-b]pyridin-2-yl)pyridin-2-amine (280 mg, 519 μmol) in THF (20 mL) was added TBAF (1 M, 1.04 mL). The mixture was stirred at 25° C. for 0.5 hr. The reaction mixture was diluted with H₂O (10 mL) and extracted with EtOAc (10 mL×3). The combined organic layers were washed with brine (30 mL), dried over Na₂SO₄, filtered and concentrated under reduced pressure. (4-(2-(2-Aminopyridin-3-yl)-5-(5-methoxypyrazin-2-yl)-3H-imidazo[4,5-b]pyridin-3-yl)phenyl)methanol (210 mg, yield: 77%) was obtained as a yellow solid. MS: m/z=426.1 [M+H]⁺.

### Step 4: 3-(3-(4-(Chloromethyl)phenyl)-5-(5-methoxypyrazin-2-yl)-3H-imidazo[4,5-b]pyridin-2-yl)pyridin-2-amine

To a solution of (4-(2-(2-aminopyridin-3-yl)-5-(5-methoxypyrazin-2-yl)-3H-imidazo[4,5-b]pyridin-3-yl)phenyl)methanol (210 mg, 486 μmol) in CH₂Cl₂ (10 mL) was added SOCl₂ (163 mg, 1.37 mmol). The mixture was stirred at 40° C. for 1 hr. The mixture was concentrated under reduced pressure to give 3-(3-(4-(chloromethyl)phenyl)-5-(5-methoxypyrazin-2-yl)-3H-imidazo[4,5-b]pyridin-2-yl)pyridin-2-amine (Intermediate 238, 250 mg, HCl salt, yield: 74%) as a yellow solid. MS: m/z=444.1 [M+H]⁺.

### Intermediate 239: 3-(3-(4-(Chloromethyl)phenyl)-5-(5-ethoxypyridin-2-yl)-3H-imidazo[4,5-b]pyridin-2-yl)pyridin-2-amine

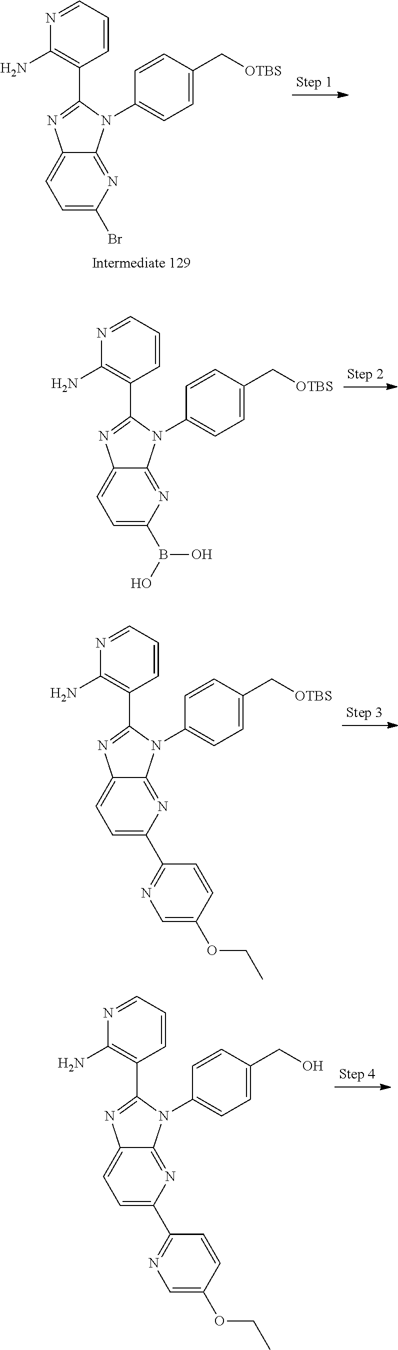

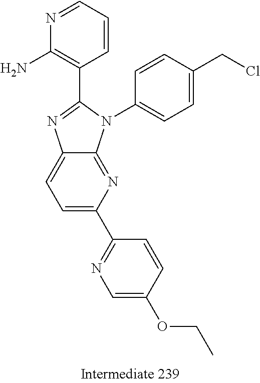 [SMILES: CCOc1ccc(-c2ccc3nc(-c4cccnc4N)n(-c4ccc(CCl)cc4)c3n2)nc1]

### Step 1: (2-(2-Aminopyridin-3-yl)-3-(4-(((tert-butyldimethylsilyl)oxy)methyl)phenyl)-3H-imidazo[4,5-b]pyridin-5-yl)boronic acid

A mixture of Intermediate 129 (1 g, 1.96 mmol), 4,4,4′,4′,5,5,5′,5′-octamethyl-2,2′-bi(1,3,2-dioxaborolane) (995 mg, 3.92 mmol), KOAc (577 mg, 5.88 mmol) and Pd(dppf)Cl₂ (143 mg, 196 μmol) in 1,4-dioxane (10 mL) was degassed and purged with N₂ three times. The mixture was stirred at 80° C. for 4 hr under N₂ atmosphere. (2-(2-Aminopyridin-3-yl)-3-(4-(((tert-butyldimethylsilyl)oxy)methyl)phenyl)-3H-imidazo[4,5-b]pyridin-5-yl)boronic acid was obtained as a black liquid, which was used in the next step without work-up and purification. MS: m/z=476.1 [M+H]⁺.

### Step 2: 3-(3-(4-(((tert-Butyldimethylsilyl)oxy)methyl)phenyl)-5-(5-ethoxypyridin-2-yl)-3H-imidazo[4,5-b]pyridin-2-yl)pyridin-2-amine

A mixture of (2-(2-aminopyridin-3-yl)-3-(4-(((tert-butyldimethylsilyl)oxy)methyl)phenyl)-3H-imidazo[4,5-b]pyridin-5-yl)boronic acid (465 mg, 978 μmol), 2-bromo-5-ethoxypyridine (217 mg, 1.08 mmol), Pd(dppf)Cl₂ (71.6 mg, 97.8 μmol) and Cs₂C03 (956 mg, 2.93 mmol) in 1,4-dioxane (5 mL) and H₂O (1 mL) was degassed and purged with N₂ three times. The mixture was stirred at 80° C. for 16 hr under N₂ atmosphere. The reaction mixture was quenched with H₂O (5 mL) and extracted with CH₂Cl₂ (15 mL×2). The combined organic layers were washed with brine (30 mL), dried over Na₂SO₄, filtered, and concentrated under reduced pressure. After purified by silica gel flash chromatography (Eluent of 15%˜43% EtOAc in petroleum ether), 3-(3-(4-(((tert-butyldimethylsilyl)oxy)methyl)phenyl)-5-(5-ethoxypyridin-2-yl)-3H-imidazo[4,5-b]pyridin-2-yl)pyridin-2-amine (360 mg, yield: 57% for two steps) was obtained as a green solid. MS: m/z=553.6 [M+H]+. ¹H NMR (400 MHz, Dimethylsulfoxide-d₆) δ 8.40-8.32 (m, 2H), 8.29-8.23 (m, 1H), 8.10 (d, J=8.8 Hz, 1H), 8.00 (d, J=4.4 Hz, 1H), 7.52-7.44 (m, 5H), 7.19 (d, J=7.6 Hz, 1H), 7.02 (br s, 2H), 6.42-6.36 (m, 1H), 4.83 (s, 2H), 4.15 (q, J=6.8 Hz, 2H), 1.36 (t, J=6.8 Hz, 3H), 0.93 (s, 9H), 0.12 (s, 6H).

### Step 3: (4-(2-(2-Aminopyridin-3-yl)-5-(5-ethoxypyridin-2-yl)-3H-imidazo[4,5-b]pyridin-3-yl)phenyl)methanol

To a solution of 3-(3-(4-(((ter/-butyldimethylsilyl)oxy)methyl)phenyl)-5-(5-ethoxypyridin-2-yl)-3H-imidazo[4,5-b]pyridin-2-yl)pyridin-2-amine (360 mg, 651 μmol) in THF (3 mL) was added TBAF (511 mg, 1.95 mmol, 1 M in THF). The mixture was stirred at 25° C. for 1 hr. The reaction mixture was quenched with H₂O (5 mL) at 25° C. and extracted with CH₂Cl₂ (15 mL×3). The combined organic layers were washed with brine (50 mL×3), dried over Na₂SO₄, filtered and concentrated under reduced pressure. After triturated with EtOAc (10 mL) at 25° C. for 10 min, (4-(2-(2-aminopyridin-3-yl)-5-(5-ethoxypyridin-2-yl)-3H-imidazo[4,5-b]pyridin-3-yl)phenyl)methanol (200 mg, yield: 67%) was obtained as a black solid. MS: m/z=439.1 [M+H]⁺. ¹H NMR (400 MHz, Dimethylsulfoxide-d₆) δ 8.39-8.32 (m, 2H), 8.26 (d, J=8.4 Hz, 1H), 8.11 (d, J=8.8 Hz, 1H), 8.00 (d, J=4.0 Hz, 1H), 7.52-7.44 (m, 5H), 7.22 (d, J=7.6 Hz, 1H), 6.98 (br s, 2H), 6.42 (dd, J=7.6, 5.2 Hz, 1H), 5.37 (t, J=5.6 Hz, 1H), 4.61 (d, J=5.6 Hz, 2H), 4.15 (q, J=6.8 Hz, 2H), 1.36 (t, J=6.8 Hz, 3H).

### Step 4: 3-(3-(4-(Chloromethyl)phenyl)-5-(5-ethoxypyridin-2-yl)-3H-imidazo[4,5-b]pyridin-2-yl)pyridin-2-amine

To a solution of (4-(2-(2-aminopyridin-3-yl)-5-(5-ethoxypyridin-2-yl)-3H-imidazo[4,5-b]pyridin-3-yl)phenyl)methanol (200 mg, 456 μmol) in CH₂Cl₂ (5 mL) was added SOCl₂ (163 mg, 1.37 mmol). The mixture was stirred at 25° C. for 0.5 hr. The reaction was concentrated under reduced pressure to give 3-(3-(4-(chloromethyl)phenyl)-5-(5-ethoxypyridin-2-yl)-3H-imidazo[4,5-b]pyridin-2-yl)pyridin-2-amine (Intermediate 239, 225 mg, HCl salt) as a brown solid. MS: m/z=457.0 [M+H]⁺. ¹H NMR (400 MHz, Methanol-d₄) δ 8.73 (d, J=9.6 Hz, 1H), 8.55 (d, J=8.4 Hz, 1H), 8.45 (d, J=2.8 Hz, 1H), 8.39 (d, J=8.4 Hz, 1H), 8.32 (dd, J=9.2, 2.8 Hz, 1H), 8.05 (dd, J=6.4, 1.6 Hz, 1H), 7.84 (dd, J=7.6, 1.2 Hz, 1H), 7.76-7.71 (m, 2H), 7.66-7.60 (m, 2H), 6.90-6.84 (m, 1H), 4.80 (s, 2H), 4.37 (q, J=7.2 Hz, 2H), 1.52 (t, J=7.2 Hz, 3H).

### Intermediate 240: 3-(3-(4-(Chloromethyl)phenyl)-5-(2-methyl-2H-1,2,3-triazol-4-yl)-3H-imidazo[4,5-b]pyridin-2-yl)pyrazin-2-amine

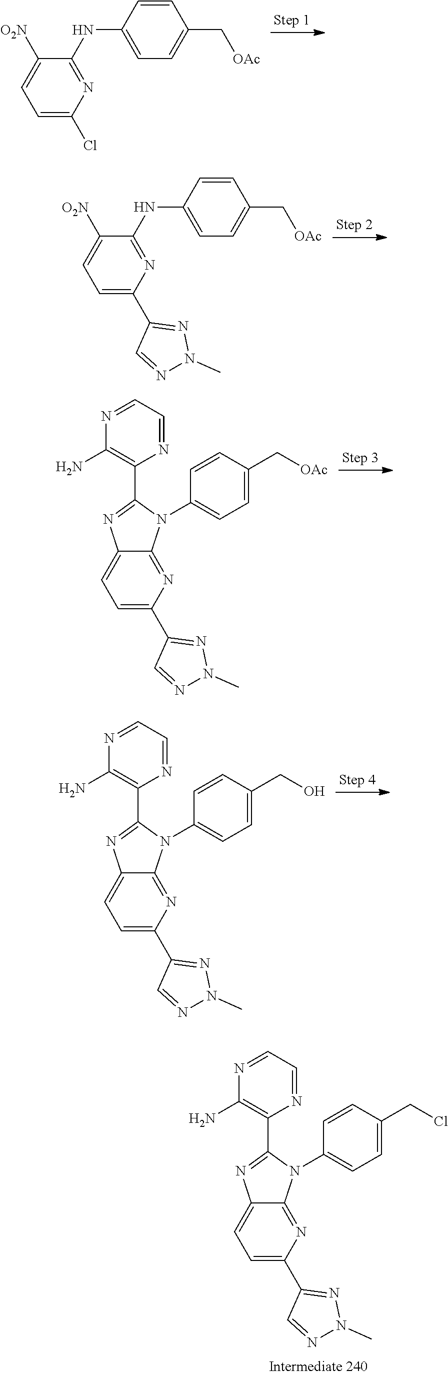

### Step 1: 4-((6-(2-Methyl-2H-1,2,3-triazol-4-yl)-3-nitropyridin-2-yl)amino)benzyl acetate

A mixture of 4-((6-chloro-3-nitropyridin-2-yl)amino)benzyl acetate (refer to Intermediate 178 for detail procedures, 515 mg, 1.60 mmol), (2-methyl-2H-1,2,3-triazol-4-yl)boronic acid (203 mg, 1.60 mmol), Cs₂CO₃ (1.56 g, 4.80 mmol) and Pd(dppf)Cl₂ (117 mg, 160 μmol) in 1,4-dioxane (6 mL) and H₂O (1.2 mL) was degassed and purged with N₂ three times. The mixture was stirred at 80° C. for 3 hr under N₂ atmosphere. The reaction mixture was quenched with H₂O (10 mL) at 25° C., diluted with CH₂Cl₂ (10 mL) and extracted with CH₂Cl₂ (15 mL×2). The combined organic layers were washed with brine (30 mL), dried over anhydrous Na₂SO₄, filtered, and concentrated under reduced pressure. After purified by silica gel flash chromatography (Eluent of 5%˜30% EtOAc in petroleum ether), 4-((6-(2-methyl-2H-1,2,3-triazol-4-yl)-3-nitropyridin-2-yl)amino)benzyl acetate (450 mg, yield: 73%) was obtained as a red solid. MS: m/z=368.9 [M+H]⁺. ¹H NMR (400 MHz, Dimethylsulfoxide-d₆) δ 10.16 (s, 1H), 8.65 (d, J=8.8 Hz, 1H), 8.18 (s, 1H), 7.82 (d, J=8.4 Hz, 2H), 7.50-7.41 (m, 3H), 5.08 (s, 2H), 4.27 (s, 3H), 2.08 (s, 3H).

### Step 2: 4-(2-(3-Aminopyrazin-2-yl)-5-(2-methyl-2H-1,2,3-triazol-4-yl)-3H-imidazo[4,5-b]pyridin-3-yl)benzyl acetate

To a solution of 4-((6-(2-methyl-2H-1,2,3-triazol-4-yl)-3-nitropyridin-2-yl)amino)benzyl acetate (415 mg, 1.13 mmol) in DMSO (5 mL) was added Na₂S₂O₄ (892 mg, 4.51 mmol, 88% purity) and 3-aminopyrazine-2-carbaldehyde (166 mg, 1.35 mmol). The mixture was stirred at 100° C. for 3 hr. The reaction mixture was quenched with H₂O (30 mL) at 25° C. and diluted with CH₂Cl₂ (20 mL) and extracted with CH₂Cl₂ (15 mL×2). The combined organic layers were washed with brine (50 mL×3), dried over anhydrous Na₂SO₄, filtered, and concentrated under reduced pressure. After purified by silica gel flash chromatography (Eluent of 10%˜35% EtOAc in petroleum ether), 4-(2-(3-aminopyrazin-2-yl)-5-(2-methyl-2H-1,2,3-triazol-4-yl)-3H-imidazo[4,5-b]pyridin-3-yl)benzyl acetate (70 mg, yield: 13%) was obtained as a yellow solid. MS: m/z=442.1 [M+H]⁺. ¹H NMR (400 MHz, Chloroform-d) δ 8.15 (d, J=8.4 Hz, 1H), 8.02-7.96 (m, 3H), 7.59 (d, J=2.4 Hz, 1H), 7.49 (d, J=8.0 Hz, 2H), 7.39 (d, J=8.4 Hz, 2H), 7.18-6.98 (m, 2H), 5.24 (s, 2H), 4.25 (s, 3H), 2.18 (s, 3H).

### Step 3: (4-(2-(3-Aminopyrazin-2-yl)-5-(2-methyl-2H-1,2,3-triazol-4-yl)-3H-imidazo[4,5-b]pyridin-3-yl)phenyl)methanol

To a solution of 4-(2-(3-aminopyrazin-2-yl)-5-(2-methyl-2H-1,2,3-triazol-4-yl)-3H-imidazo[4,5-b]pyridin-3-yl)benzyl acetate (70 mg, 159 μmol) in THF (1 mL), MeOH (1 mL) and H₂O (1 mL) was added K₂CO₃ (65.8 mg, 476 μmol). The mixture was stirred at 25° C. for 1 hr. The reaction mixture was quenched with H₂O (10 mL) at 25° C., diluted with CH₂Cl₂ (10 mL) and extracted with CH₂Cl₂ (15 mL×3). The combined organic layers were washed with brine (30 mL), dried over Na₂SO₄, filtered and concentrated under reduced pressure. (4-(2-(3-Aminopyrazin-2-yl)-5-(2-methyl-2H-1,2,3-triazol-4-yl)-3H-imidazo[4,5-b]pyridin-3-yl)phenyl)methanol (70 mg) was obtained as a yellow solid. MS: m/z=400.1 [M+H]⁺. ¹H NMR (400 MHz, Chloroform-d) 68.15 (d, J=8.4 Hz, 1H), 8.03-7.95 (m, 3H), 7.59 (d, J=2.4 Hz, 1H), 7.51 (d, J=8.4 Hz, 2H), 7.38 (d, J=8.0 Hz, 2H), 7.14-6.90 (m, 2H), 4.85 (s, 2H), 4.24 (s, 3H).

### Step 4: 3-(3-(4-(Chloromethyl)phenyl)-5-(2-methyl-2H-1,2,3-triazol-4-yl)-3H-imidazo[4,5-b]pyridin-2-yl)pyrazin-2-amine

To a solution of (4-(2-(3-aminopyrazin-2-yl)-5-(2-methyl-2H-1,2,3-triazol-4-yl)-3H-imidazo[4,5-b]pyridin-3-yl)phenyl)methanol (70 mg, 175 μmol) in CH₂Cl₂ (4 mL) was added SOCl₂ (62.6 mg, 526 μmol). The mixture was stirred at 25° C. for 0.5 hr. The reaction was concentrated under reduced pressure to give 3-(3-(4-(chloromethyl)phenyl)-5-(2-methyl-2H-1,2,3-triazol-4-yl)-3H-imidazo[4,5-b]pyridin-2-yl)pyrazin-2-amine (Intermediate 240, 80 mg, HCl salt) as a yellow solid. MS: m/z=418.1 [M+H]⁺. ¹H NMR (400 MHz, Methanol-d₄) δ 8.32 (d, J=8.4 Hz, 1H), 8.10 (d, J=8.4 Hz, 1H), 7.98 (s, 1H), 7.90 (d, J=2.8 Hz, 1H), 7.69 (d, J=2.8 Hz, 1H), 7.61 (d, J=8.4 Hz, 2H), 7.45 (d, J=8.4 Hz, 2H), 4.79 (s, 2H), 4.22 (s, 3H).

### Intermediate 241: 2-(2-(2-Aminopyridin-3-yl)-3-(4-(chloromethyl)phenyl)-3H-imidazo[4,5-b]pyridin-5-yl)isonicotinonitrile

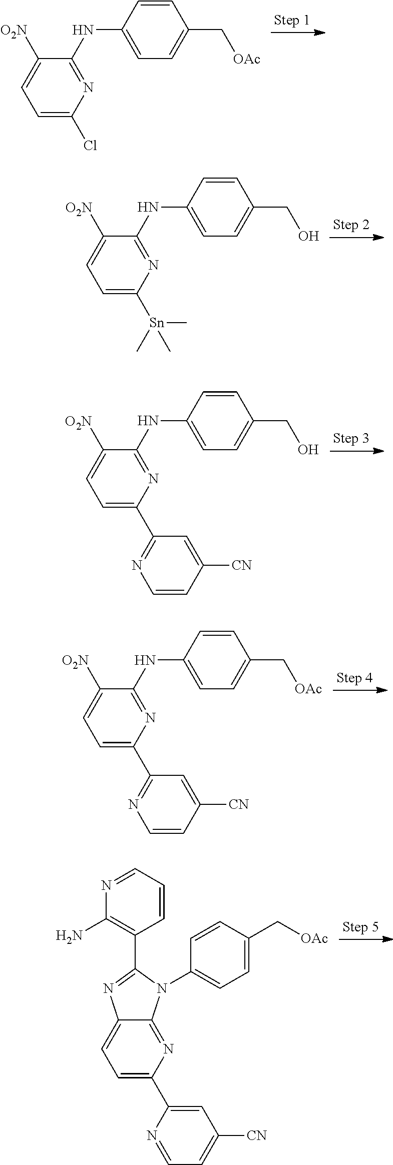

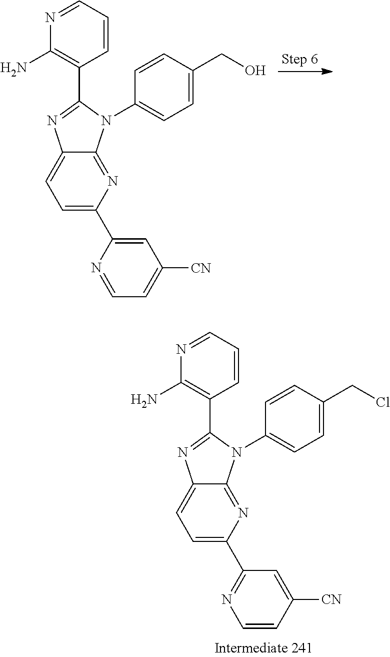 [SMILES: N#Cc1ccnc(-c2ccc3nc(-c4cccnc4N)n(-c4ccc(CO)cc4)c3n2)c1 ; N#Cc1ccnc(-c2ccc3nc(-c4cccnc4N)n(-c4ccc(CCl)cc4)c3n2)c1]

### Step 1: (4-((3-Nitro-6-(trimethylstannyl)pyridin-2-yl)amino)phenyl)methanol

To a solution of 4-((6-chloro-3-nitropyridin-2-yl)amino)benzyl acetate (refer to Intermediate 178 for detail procedures, 5 g, 15.5 mmol), Pd(PPh₃)₄ (1.8 g, 1.55 mmol) and 1,1,1,2,2,2-hexamethyldistannane (14.4 g, 44 mmol) in 1,4-dioxane (50 mL) was degassed and purged with N₂ three times. The mixture was stirred at 125° C. for 2 hr under N₂ atmosphere. (4-((3-Nitro-6-(trimethylstannyl)pyridin-2-yl)amino)phenyl)methanol was obtained as a black liquid, which was used in the next step without work-up and purification. MS: m/z=409.9 [M+H]⁺.

### Step 2: 6′-((4-(Hydroxymethyl)phenyl)amino)-5′-nitro-[2,2′-bipyridine]-4-carbonitrile

A mixture of (4-((3-nitro-6-(trimethylstannyl)pyridin-2-yl)amino)phenyl)methanol (6.34 g, 15.5 mmol), 2-bromoisonicotinonitrile (2.84 g, 15.5 mmol), Pd(PPh₃)₄ (1.79 g, 1.55 mmol) in 1,4-dioxane (100 mL) was degassed and purged with N₂ three times. The mixture was stirred at 100° C. for 16 hr under N₂ atmosphere. The reaction was concentrated under reduced pressure. After purified by silica gel flash chromatography (Eluent of 5˜100% EtOAc in petroleum ether), 6′-((4-(hydroxymethyl)phenyl)amino)-5′-nitro-[2,2′-bipyridine]-4-carbonitrile (1.4 g, yield: 28% for two steps) was obtained as a black solid. MS: m/z=348.0 [M+H]⁺.

### Step 3: 4-((4′-Cyano-5-nitro-[2,2′-bipyridin]-6-yl)amino)benzyl acetate

To a solution of 6′-((4-(hydroxymethyl)phenyl)amino)-5′-nitro-[2,2′-bipyridine]-4-carbonitrile (1.3 g, 3.74 mmol) in CH₂Cl₂ (15 mL) were added TEA (1.14 g, 11.2 mmol), DMAP (45.7 mg, 374 μmol), and Ac₂O (573 mg, 5.61 mmol). The mixture was stirred at 25° C. for 1 hr. The reaction mixture was quenched with H₂O (30 mL) at 25° C., diluted with CH₂Cl₂ (30 mL) and extracted with CH₂Cl₂ (50 mL×2). The combined organic layers were washed with brine (50 mL), dried over Na₂SO₄, filtered, and concentrated under reduced pressure. After purified by silica gel flash chromatography (Eluent of 10˜30% EtOAc in petroleum ether), 4-((4′-cyano-5-nitro-[2,2′-bipyridin]-6-yl)amino)benzyl acetate (1.4 g, yield: 40%) was obtained as a black solid. MS: m/z=390.0 [M+H]⁺.

### Step 4: 4-(2-(2-Aminopyridin-3-yl)-5-(4-cyanopyridin-2-yl)-3H-imidazo[4,5-b]pyridin-3-yl)benzyl acetate

To a solution of 4-((4′-cyano-5-nitro-[2,2′-bipyridin]-6-yl)amino)benzyl acetate (1.2 g, 3.08 mmol) in DMSO (10 mL) were added Na₂S₂O₄ (2.38 g, 12.3 mmol, 90% purity) and 2-aminonicotinaldehyde (452 mg, 3.70 mmol). The mixture was stirred at 100° C. for 2 hr. The reaction mixture was quenched with H₂O (50 mL) at 25° C., diluted with CH₂Cl₂ (30 mL) and extracted with CH₂C12 (30 mL×3). The combined organic layers were washed with brine (50 mL×3), dried over Na₂SO₄, filtered, and concentrated under reduced pressure. After purified by silica gel flash chromatography (Eluent of 10%˜70% EtOAc in petroleum ether), 4-(2-(2-aminopyridin-3-yl)-5-(4-cyanopyridin-2-yl)-3H-imidazo[4,5-b]pyridin-3-yl)benzyl acetate (80 mg, yield: 5.6%) was obtained as a yellow solid. MS: m/z=462.0 [M+H]+. ¹H NMR (400 MHz, Chloroform-d) δ 8.81 (d, J=4.8 Hz, 1H), 8.55-8.52 (m, 2H), 8.21 (d, J=8.4 Hz, 1H), 8.10 (dd, J=4.8, 1.6 Hz, 1H), 7.60-7.56 (m, 2H), 7.47-7.44 (m, 3H), 7.12 (dd, J=7.6, 1.6 Hz, 1H), 6.66 (br s, 2H), 6.41 (dd, J=8.0, 4.8 Hz, 1H), 5.27 (s, 2H), 2.20 (s, 3H).

### Step 5: 2-(2-(2-Aminopyridin-3-yl)-3-(4-(hydroxymethyl)phenyl)-3H-imidazo[4,5-b]pyridin-5-yl)isonicotinonitrile

To a solution of 4-(2-(2-aminopyridin-3-yl)-5-(4-cyanopyridin-2-yl)-3H-imidazo[4,5-b]pyridin-3-yl)benzyl acetate (80 mg, 173 μmol) in THF (2 mL), MeOH (2 mL) and H₂O (1 mL) was added the K₂CO₃ (71.9 mg, 520 μmol). The mixture was stirred at 25° C. for 2 hr. The reaction mixture was diluted with H₂O (10 mL) and extracted with CH₂Cl₂ (15 mL×2). The combined organic layers were washed with brine (15 mL), dried over Na₂SO₄, filtered and concentrated under reduced pressure. 2-(2-(2-Aminopyridin-3-yl)-3-(4-(hydroxymethyl)phenyl)-3H-imidazo[4,5-b]pyridin-5-yl)isonicotinonitrile (60 mg) was obtained as a yellow solid. MS: m/z=420.0 [M+H]⁺.

### Step 6: 2-(2-(2-Aminopyridin-3-yl)-3-(4-(chloromethyl)phenyl)-3H-imidazo[4,5-b]pyridin-5-yl)isonicotinonitrile

To a solution of 2-(2-(2-aminopyridin-3-yl)-3-(4-(hydroxymethyl)phenyl)-3H-imidazo[4,5-b]pyridin-5-yl)isonicotinonitrile (60 mg, 143 μmol) in CH₂Cl₂ (5 mL) was added SOCl₂ (51.1 mg, 429 μmol). The mixture was stirred at 40° C. for 0.4 hr. The reaction was concentrated under reduced pressure to give 2-(2-(2-aminopyridin-3-yl)-3-(4-(chloromethyl)phenyl)-3H-imidazo[4,5-b]pyridin-5-yl)isonicotinonitrile (Intermediate 241, 67.9 mg, HCl salt) was obtained as a yellow solid. MS: m/z=437.9 [M+H]⁺.

### Intermediate 242: 3-(3-(4-(Chloromethyl)phenyl)-5-(5-methoxypyridin-2-yl)-3H-imidazo[4,5-b]pyridin-2-yl)pyridin-2-amine

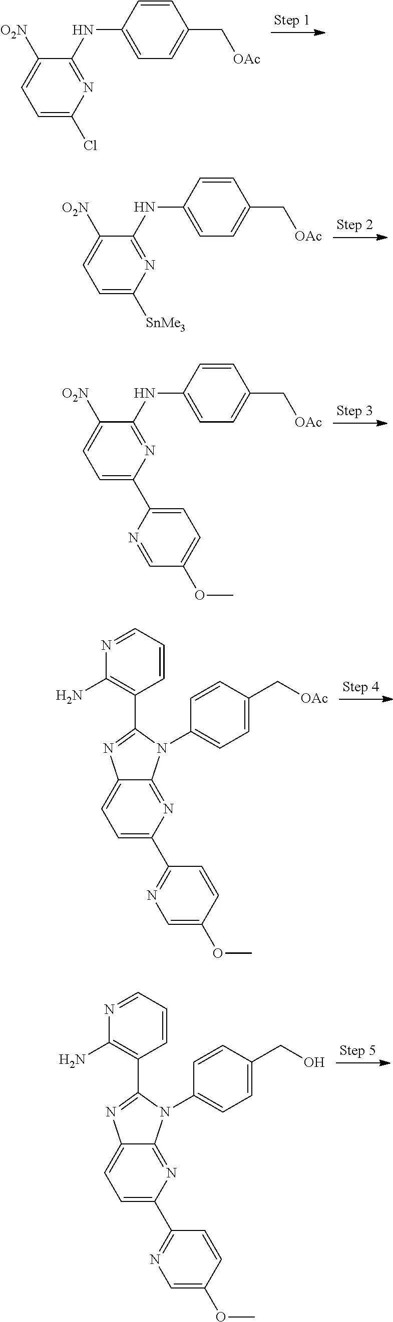

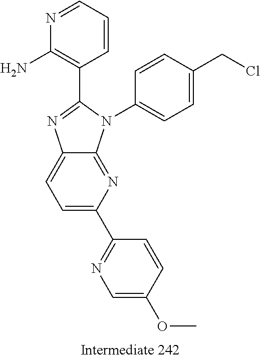 [SMILES: COc1ccc(-c2ccc3nc(-c4cccnc4N)n(-c4ccc(CCl)cc4)c3n2)nc1]

### Step 1: 4-((3-Nitro-6-(trimethylstannyl)pyridin-2-yl)amino)benzyl acetate

A mixture of 4-((6-chloro-3-nitropyridin-2-yl)amino)benzyl acetate (refer to Intermediate 178 for detail procedures, 5 g, 15.5 mmol), (Me₃Sn)₂ (10.8 g, 33.0 mmol), Pd(PPh₃)₄ (287 mg, 777 μmol) in 1,4-dioxane (50 mL) was degassed and purged with N₂ three times, and then the mixture was stirred at 90° C. for 3 hr under N₂ atmosphere. 4-((3-Nitro-6-(trimethylstannyl)pyridin-2-yl)amino)benzyl acetate was obtained as a black brown liquid. The liquid was directly used in the next step. MS: m/z=451.9 [M+H]⁺.

### Step 2: 4-((5′-Methoxy-5-nitro-[2,2′-bipyridin]-6-yl)amino)benzyl acetate

A mixture of 4-((3-nitro-6-(trimethylstannyl)pyridin-2-yl)amino)benzyl acetate (6.99 g, 15.5 mmol), 2-bromo-5-methoxypyridine (2.92 g, 15.5 mmol), Pd(PPh₃)₄ (115 mg, 311 μmol) in 1,4-dioxane (50 mL) was degassed and purged with N₂ three times. The mixture was stirred at 100° C. for 50 hr under N₂ atmosphere. The mixture was diluted with H₂O (100 mL) and extracted with CH₂Cl₂ (100 mL×3). The combined organic layers were washed with brine (50 mL×2), dried over Na₂SO₄, filtered and concentrated under reduced pressure. After purified by silica gel flash chromatography (Eluent of 0˜19% EtOAc in petroleum ether), 4-((5′-methoxy-5-nitro-[2,2′-bipyridin]-6-yl)amino)benzyl acetate (560 mg, yield: 5.8% for two steps) was obtained as an orange red solid. MS: m/z=395.1 [M+H]⁺.

### Step 3: 4-(2-(2-Aminopyridin-3-yl)-5-(5-methoxypyridin-2-yl)-3H-imidazo[4,5-b]pyridin-3-yl)benzyl acetate

A mixture of 4-((5′-methoxy-5-nitro-[2,2′-bipyridin]-6-yl)amino)benzyl acetate (560 mg, 1.42 mmol), 2-aminonicotinaldehyde (208 mg, 1.70 mmol) and Na₂S₂O₄ (989 mg, 5.68 mmol) in DMSO (5 mL) was degassed and purged with N₂ three times. The mixture was stirred at 100° C. for 3 hr under N₂ atmosphere. The reaction mixture was diluted with H₂O (500 mL) and extracted with EtOAc (500 mL×2). The combined organic layers were washed with brine (200 mL), dried over Na₂SO₄, filtered and concentrated under reduced pressure. After purified by silica gel flash chromatography (Eluent of 0˜43% EtOAc in petroleum ether), 4-(2-(2-aminopyridin-3-yl)-5-(5-methoxypyridin-2-yl)-3H-imidazo[4,5-b]pyridin-3-yl)benzyl acetate (200 mg, yield: 21%) was obtained as a light yellow solid. MS: m/z=467.1 [M+H]⁺.

### Step 4: (4-(2-(2-Aminopyridin-3-yl)-5-(5-methoxypyridin-2-yl)-3H-imidazo[4,5-b]pyridin-3-yl)phenyl)methanol

To a solution of 4-(2-(2-aminopyridin-3-yl)-5-(5-methoxypyridin-2-yl)-3H-imidazo[4,5-b]pyridin-3-yl)benzyl acetate (200 mg, 429 μmol) in THF (1 mL), MeOH (1 mL) and H₂O (0.5 mL) was added K₂CO₃ (178 mg, 1.29 mmol). The mixture was stirred at 25° C. for 0.5 hr. The reaction mixture was diluted with H₂O (50 mL) and extracted with EtOAc (50 mL×2). The combined organic layers were washed with brine (15 mL), dried over Na₂SO₄, filtered and concentrated under reduced pressure. (4-(2-(2-Aminopyridin-3-yl)-5-(5-methoxypyridin-2-yl)-3H-imidazo[4,5-b]pyridin-3-yl)phenyl)methanol (160 mg) was obtained as a light yellow solid. MS: m/z=425.1 [M+H]⁺. ¹H NMR (400 MHz, Chloroform-d) δ 8.85 (d, J=7.6 Hz, 1H), 8.73-8.54 (m, 1H), 8.43 (d, J=7.2 Hz, 2H), 7.95-7.83 (m, 2H), 7.72 (s, 2H), 7.65-7.53 (m, 3H), 7.22-7.11 (m, 1H), 6.62 (d, J=2.0 Hz, 1H), 4.73 (s, 2H), 4.08 (s, 3H).

### Step 5: 3-(3-(4-(Chloromethyl)phenyl)-5-(5-methoxypyridin-2-yl)-3H-imidazo[4,5-b]pyridin-2-yl)pyridin-2-amine

To a solution of (4-(2-(2-aminopyridin-3-yl)-5-(5-methoxypyridin-2-yl)-3H-imidazo[4,5-b]pyridin-3-yl)phenyl)methanol (160 mg, 377 μmol) in CH₂Cl₂ (2 mL) was added SOCl₂ (51.1 mg, 429 μmol). The mixture was stirred at 40° C. for 0.5 hr and concentrated under reduced pressure to give 3-(3-(4-(chloromethyl)phenyl)-5-(5-methoxypyridin-2-yl)-3H-imidazo[4,5-b]pyridin-2-yl)pyridin-2-amine (Intermediate 242, 160 mg, HCl salt) as a light yellow solid. MS: m/z=443.1 [M+H]⁺.

### Intermediate 243: 2-(2-Aminopyridin-3-yl)-1-(4-(chloromethyl)phenyl)-1H-imidazo[4,5-b]pyrazine-5,6-dicarbonitrile

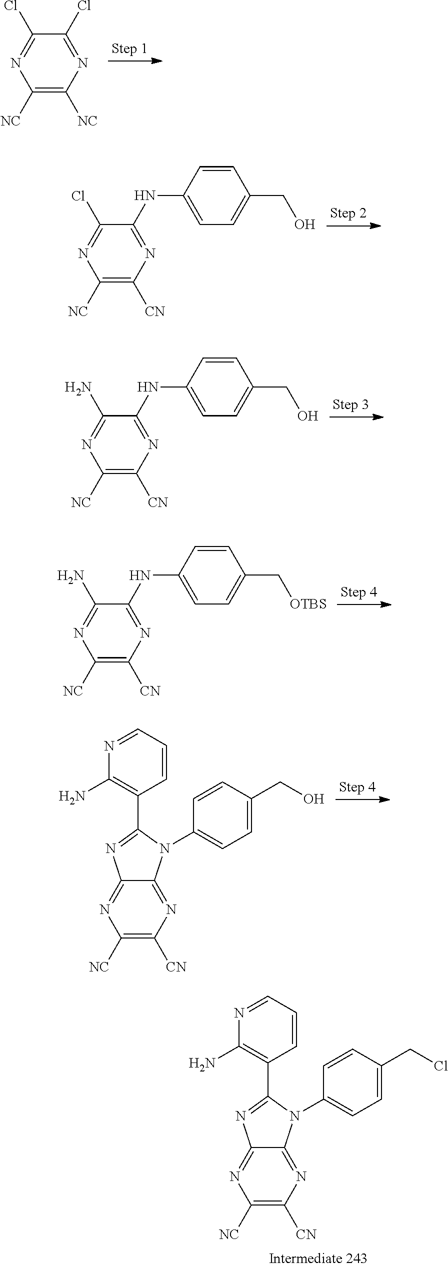

### Step 1: 5-Chloro-6-((4-(hydroxymethyl)phenyl)amino)pyrazine-2,3-dicarbonitrile

To a solution of 5,6-dichloropyrazine-2,3-dicarbonitrile (10 g, 50.3 mmol) in THF (100 mL) was added (4-aminophenyl)methanol (12.4 g, 101 mmol) in THF (100 mL) dropwise slowly under ice bath. The mixture was degassed and purged with N₂ three times and stirred at 0° C. for 3 hr under N₂. The reaction mixture was filtered and concentrated under reduced pressure. 5-Chloro-6-((4-(hydroxymethyl)phenyl)amino)pyrazine-2,3-dicarbonitrile (13.2 g, yield: 90%) was obtained as a yellow solid. ¹H NMR (400 MHz, Dimethylsulfoxide-d₆) δ 10.27 (br s, 1H), 7.55 (d, J=8.4 Hz, 2H), 7.44 (d, J=8.4 Hz, 2H), 4.57 (s, 2H).

### Step 2: 5-Amino-6-((4-(hydroxymethyl)phenyl)amino)pyrazine-2,3-dicarbonitrile

To a solution of 5-chloro-6-((4-(hydroxymethyl)phenyl)amino)pyrazine-2,3-dicarbonitrile (13 g, 45.5 mmol) in DMF (150 mL) was stirred at 0° C. under NH₃ atmosphere for 0.5 hr. The reaction mixture was quenched with H₂O (500 mL) and filtered. The filtered cake was dried under reduced pressure to give 5-amino-6-((4-(hydroxymethyl)phenyl)amino)pyrazine-2,3-dicarbonitrile (10.7 g, yield: 85%) as a yellow solid. MS: m4=267.0 [M+H]⁺. ¹H NMR (400 MHz, Dimethylsulfoxide-d₆) δ 9.22 (br s, 1H), 8.02-7.81 (m, 2H), 7.59 (d, J=8.4 Hz, 2H), 7.34 (d, J=8.4 Hz, 2H), 5.17 (t, J=5.6 Hz, 1H), 4.48 (d, J=5.6 Hz, 2H).

### Step 3: 5-Amino-6-((4-(((tert-butyldimethylsilyl)oxy)methyl)phenyl)amino)pyrazine-2,3-dicarbonitrile

To a solution of 5-amino-6-((4-(hydroxymethyl)phenyl)amino)pyrazine-2,3-dicarbonitrile (1 g, 3.76 mmol) in CH₂Cl₂ (10 mL) were added TBSCl (679 mg, 4.51 mmol), TEA (1.57 mL, 11.3 mmol) and DMAP (45.9 mg, 376 μmol). The mixture was degassed and purged with N₂ three times and stirred at 25° C. for 1 hr under N₂. The reaction mixture was diluted with CH₂Cl₂ (100 mL), washed with brine (100 mL×2), separated, dried over Na₂SO₄, filtered and concentrated under reduced pressure. After purified by silica gel flash chromatography (MeOH in CH₂Cl₂=1 to 10%), 5-amino-6-((4-(((tert-butyldimethylsilyl)oxy)methyl)phenyl)amino)pyrazine-2,3-dicarbonitrile (680 mg, yield: 47%) was obtained as a red solid. ¹H NMR (400 MHz, Dimethylsulfoxide-d₆) 9.33 (br s, 1H), 7.99 (br s, 2H), 7.64 (d, J=8.4 Hz, 2H), 7.33 (d, J=8.4 Hz, 2H), 4.69 (s, 2H), 0.90 (s, 9H), 0.08 (s, 6H).

### Step 4: 2-(2-Aminopyridin-3-yl)-1-(4-(hydroxymethyl)phenyl)-1H-imidazo[4,5-b]pyrazine-5,6-dicarbonitrile

To a solution of 5-amino-6-((4-(((tert-butyldimethylsilyl)oxy)methyl)phenyl)amino)pyrazine-2,3-dicarbonitrile (1.5 g, 3.94 mmol) in DMSO (100 mL) were added Na₂S₂O₄ (2.06 g, 11.8 mmol) and 2-aminonicotinaldehyde (481 mg, 3.94 mmol). The mixture was degassed, purged with N₂ three times, and stirred at 100° C. for 2 hr under N₂. The reaction mixture was diluted with CH₂Cl₂ (500 mL) and filtered. The filtrate was washed with brine (300 mL×3), separated, dried over Na₂SO₄, filtered and concentrated under reduced pressure. After purified by silica gel flash chromatography (Ethyl acetate in petroleum ether=1% to 100%), 2-(2-aminopyridin-3-yl)-1-(4-(hydroxymethyl)phenyl)-1H-imidazo[4,5-b]pyrazine-5,6-dicarbonitrile (366 mg, yield: 23%) was obtained as a yellow solid. MS: m/z=369.0 [M+H]+. ¹H NMR (400 MHz, Dimethylsulfoxide-d₆) δ 8.13 (dd, J=4.8, 2.0 Hz, 1H), 7.56-7.51 (m, 2H), 7.48-7.43 (m, 2H), 7.36 (dd, J=8.0, 2.0 Hz, 1H), 7.18 (br s, 2H), 6.49 (dd, J=8.0, 4.8 Hz, 1H), 5.39 (t, J=5.6 Hz, 1H), 4.60 (d, J=5.4 Hz, 2H).

### Step 5: 2-(2-Aminopyridin-3-yl)-1-(4-(chloromethyl)phenyl)-1H-imidazo[4,5-b]pyrazine-5,6-dicarbonitrile

To a solution of 2-(2-aminopyridin-3-yl)-1-(4-(hydroxymethyl)phenyl)-1H-imidazo[4,5-b]pyrazine-5,6-dicarbonitrile (366 mg, 894 μmol) in CH₂Cl₂ (5 mL) was added SOCl₂ (649 μL, 8.94 mmol) dropwise. The mixture was degassed and purged with N₂ three times and stirred at 25° C. for 1 hr under N₂. The reaction mixture was concentrated under reduced pressure to give 2-(2-aminopyridin-3-yl)-1-(4-(chloromethyl)phenyl)-1H-imidazo[4,5-b]pyrazine-5,6-dicarbonitrile (Intermediate 243, 379 mg, crude, HCl salt) as a yellow solid, which was used in the next step without further purification. MS: m/z=387.1 [M+H]⁺.

### Intermediate 244: 2-(2-Aminopyridin-3-yl)-3-(4-(chloromethyl)phenyl)-5-(4-fluorophenyl)-3H-imidazo[4,5-b]pyridine-6-carbonitrile

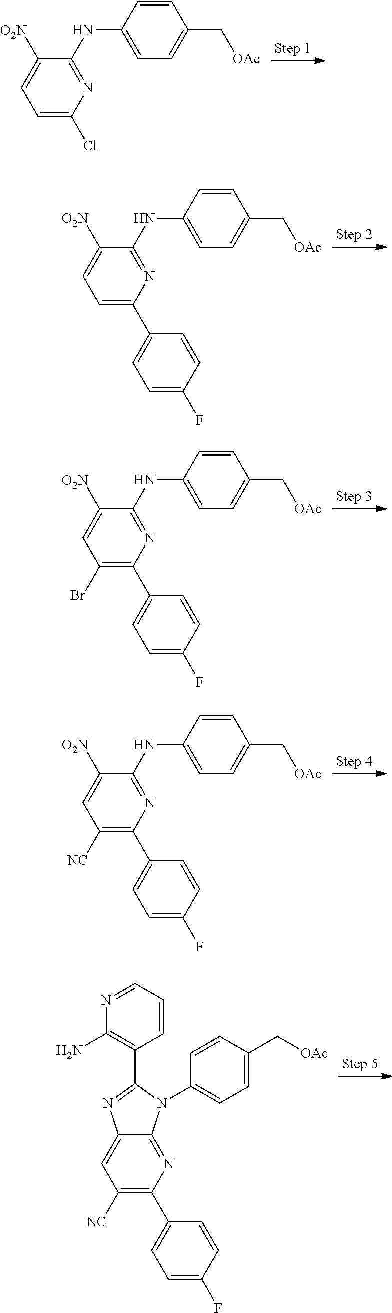

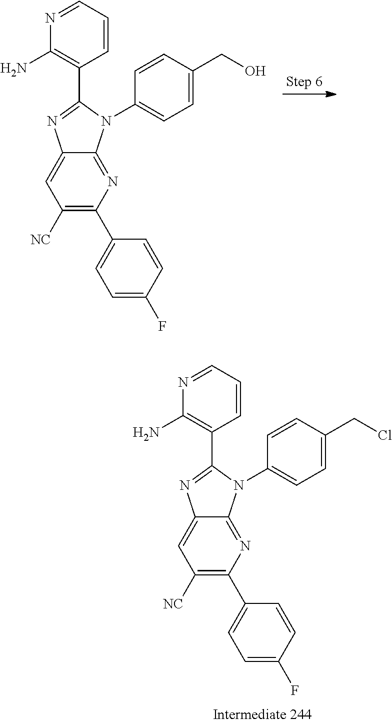 [SMILES: N#Cc1cc2nc(-c3cccnc3N)n(-c3ccc(CO)cc3)c2nc1-c1ccc(F)cc1 ; N#Cc1cc2nc(-c3cccnc3N)n(-c3ccc(CCl)cc3)c2nc1-c1ccc(F)cc1]

### Step 1: 4-((6-(4-Fluorophenyl)-3-nitropyridin-2-yl)amino)benzyl acetate

A mixture of 4-((6-chloro-3-nitropyridin-2-yl)amino)benzyl acetate (refer to Intermediate 178 for detail procedures, 500 mg, 1.55 mmol), (4-fluorophenyl)boronic acid (217 mg, 1.55 mmol), Cs₂CO₃ (1.52 g, 4.66 mmol) and Pd(dppf)Cl₂ (114 mg, 155 μmol) in 1,4-dioxane (10 mL) and H₂O (2 mL) was degassed and purged with N₂ three times. The mixture was stirred at 80° C. for 16 hr under N₂ atmosphere. The mixture was filtered and concentrated. After purified by silica gel flash chromatography (Eluent of 10%˜20% EtOAc in petroleum ether), 4-((6-(4-fluorophenyl)-3-nitropyridin-2-yl)amino)benzyl acetate (300 mg, yield: 48%) was obtained as a yellow solid. MS: m/z=381.9 [M+H]⁺. ¹H NMR (400 MHz, Dimethylsulfoxide-d₆) δ 10.12 (s, 1H), 8.61 (d, J=8.8 Hz, 1H), 8.20-8.13 (m, 2H), 7.74 (d, J=8.4 Hz, 2H), 7.59 (d, 0.1=8.8 Hz, 1H), 7.44-7.34 (m, 4H), 5.09 (s, 2H), 2.08 (s, 3H).

### Step 2: 4-((5-Bromo-6-(4-fluorophenyl)-3-nitropyridin-2-yl)amino)benzyl acetate

To a solution of 4-((6-(4-fluorophenyl)-3-nitropyridin-2-yl)amino)benzyl acetate (100 mg, 262 μmol) in DMF (2 mL) was added NBS (51.3 mg, 288 μmol). The mixture was stirred at 25° C. for 2 hr. The mixture was poured into water (10 mL) at 0° C. and filtrated. The solid was dried and purified by prep-HPLC (column: Phenomenex C18 150×25 mm×10 μm; mobile phase: [water (NH₄HCO₃)-ACN]; gradient: 65%-85% B over 11 min) to give 4-((5-bromo-6-(4-fluorophenyl)-3-nitropyridin-2-yl)amino)benzyl acetate (100 mg, yield: 41%) as a red solid. ¹H NMR (400 MHz, Dimethylsulfoxide-d₆) δ 10.02 (s, 1H), 8.80 (s, 1H), 7.80-7.75 (m, 2H), 7.67 (d, J=8.4 Hz, 2H), 7.38-7.31 (nm, 4H), 5.03 (s, 2H), 2.05 (s, 3H).

### Step 3; 4-((5-Cyano-6-(4-fluorophenyl)-3-nitropyridin-2-yl)amino)benzyl acetate

A mixture of 4-((5-bromo-6-(4-fluorophenyl)-3-nitropyridin-2-yl)amino)benzyl acetate (1.7 g, 3.69 mmol) and CuCN (1.65 g, 18.5 mmol) in DMF (20 mL) was degassed and purged with N2 three times. The mixture was stirred at 140° C. for 3 hr under N₂ atmosphere. The reaction mixture was quenched with H₂O (50 mL) at 25° C. and extracted with EtOAc (50 mL×3). The combined organic layers were washed with brine (100 mL×3), dried over Na₂SO₄, filtered, and concentrated under reduced pressure. After purified by silica gel flash chromatography (Eluent of 5%˜15% EtOAc in petroleum ether), 4-((5-cyano-6-(4-fluorophenyl)-3-nitropyridin-2-yl)amino)benzyl acetate (1.02 g, yield: 68%) was obtained as a yellow solid. MS: m/z=406.9 [M+H]⁺. ¹H NMR (400 MHz, Dimethylsulfoxide-d₆) δ 10.41 (s, 1H), 9.10 (s, 1H), 7.98-7.89 (m, 2H), 7.68 (d, J=8.8 Hz, 2H), 7.49-7.37 (m, 4H), 5.08 (s, 2H), 2.08 (s, 3H).

### Step 4: 4-(2-(2-Aminopyridin-3-yl)-6-cyano-5-(4-fluorophenyl)-3H-imidazo[4,5-b]pyridin-3-yl)benzyl acetate

To a solution of 4-((5-cyano-6-(4-fluorophenyl)-3-nitropyridin-2-yl)amino)benzyl acetate (950 mg, 2.34 mmol) in DMSO (10 mL) were added Na₂S₂O₄ (1.85 g, 9.35 mmol, 88% purity) and 2-aminonicotinaldehyde (343 mg, 2.81 mmol). The mixture was stirred at 100° C. for 4 hr. The reaction mixture was quenched with H₂O (30 mL) at 25° C., diluted with CH₂Cl₂ (30 mL), and extracted with CH₂Cl₂ (50 mL×2). The combined organic layers were washed with brine (100 mL×3), dried over Na₂SO₄, filtered, and concentrated under reduced pressure. After purified by silica gel flash chromatography (Eluent of 20%˜40% EtOAc in petroleum ether), 4-(2-(2-aminopyridin-3-yl)-6-cyano-5-(4-fluorophenyl)-3H-imidazo[4,5-b]pyridin-3-yl)benzyl acetate (300 mg, yield: 27%) was obtained as a yellow solid. MS: m/z=479.0 [M+H]⁺. ¹H NMR (400 MHz, Chloroform-d) δ 8.44 (s, 1H), 8.14-8.08 (m, 1H), 7.89 (dd, J=8.4, 5.2 Hz, 2H), 7.52 (d, J=8.0 Hz, 2H), 7.40 (d, J=8.4 Hz, 2H), 7.17 (t, J=8.4 Hz, 2H), 7.09 (d, J=7.6 Hz, 1H), 6.68 (br s, 2H), 6.40 (dd, J=8.0, 4.8 Hz, 1H), 5.21 (s, 2H), 2.16 (s, 3H).

### Step 5: 2-(2-Aminopyridin-3-yl)-5-(4-fluorophenyl)-3-(4-(hydroxymethyl)phenyl)-3H-imidazo[4,5-b]pyridine-6-carbonitrile

To a solution of 4-(2-(2-aminopyridin-3-yl)-6-cyano-5-(4-fluorophenyl)-3H-imidazo[4,5-b]pyridin-3-yl)benzyl acetate (290 mg, 606 μmol) in THF (5 mL), MeOH (5 mL) and H₂O (5 mL) was added K₂CO₃ (251 mg, 1.82 mmol). The mixture was stirred at 25° C. for 1 hr. The reaction mixture was quenched with H₂O (10 mL) at 25° C., diluted with CH₂Cl₂ (10 mL), and extracted with CH₂Cl₂ (15 mL×2). The combined organic layers were washed with brine (30 mL), dried over Na₂SO₄, filtered and concentrated under reduced pressure. 2-(2-Aminopyridin-3-yl)-5-(4-fluorophenyl)-3-(4-(hydroxymethyl)phenyl)-3H-imidazo[4,5-b]pyridine-6-carbonitrile (270 mg) was obtained as a yellow solid. MS: m/z=437.0 [M+H]+. ¹H NMR (400 MHz, Dimethylsulfoxide-d₆) δ 8.90 (s, 1H), 8.02 (dd, J=4.8, 1.6 Hz, 1H), 7.85-7.80 (m, 2H), 7.48-7.43 (m, 4H), 7.40-7.36 (m, 2H), 7.27 (dd, J=7.6, 1.6 Hz, 1H), 6.90 (br s, 2H), 6.44 (dd, J=7.6, 4.8 Hz, 1H), 5.34 (t, J=5.6 Hz, 1H), 4.57 (d, J=5.6 Hz, 2H).

### Step 6: 2-(2-Aminopyridin-3-yl)-3-(4-(chloromethyl)phenyl)-5-(4-fluorophenyl)-3H-imidazo[4,5-b]pyridine-6-carbonitrile

To a solution of 2-(2-aminopyridin-3-yl)-5-(4-fluorophenyl)-3-(4-(hydroxymethyl)phenyl)-3H-imidazo[4,5-b]pyridine-6-carbonitrile (270 mg, 619 μmol) in CH₂Cl₂ (10 mL) was added SOCl₂ (221 mg, 1.86 mmol) at 0° C. The mixture was stirred at 40° C. for 0.3 hr. The reaction was concentrated under reduced pressure to give 2-(2-aminopyridin-3-yl)-3-(4-(chloromethyl)phenyl)-5-(4-fluorophenyl)-3H-imidazo[4,5-b]pyridine-6-carbonitrile (Intermediate 244, 304 mg, HCl salt) as a yellow solid. MS: m/z=455.0 [M+H]⁺. ¹H NMR (400 MHz, Methanol-d₄) δ 8.76 (s, 1H), 8.04 (dd, J=6.4, 1.2 Hz, 1H), 7.93-7.85 (m, 3H), 7.67 (d, J=8.4 Hz, 2H), 7.55 (d, J=8.4 Hz, 2H), 7.24-7.22 (m, 2H), 6.92-6.84 (m, 1H), 4.76 (s, 2H).

### Intermediate 245: 3-(6-Bromo-3-(4-(chloromethyl)phenyl)-5-(4-fluorophenyl)-3H-imidazo[4,5-b]pyridin-2-yl)pyridin-2-amine

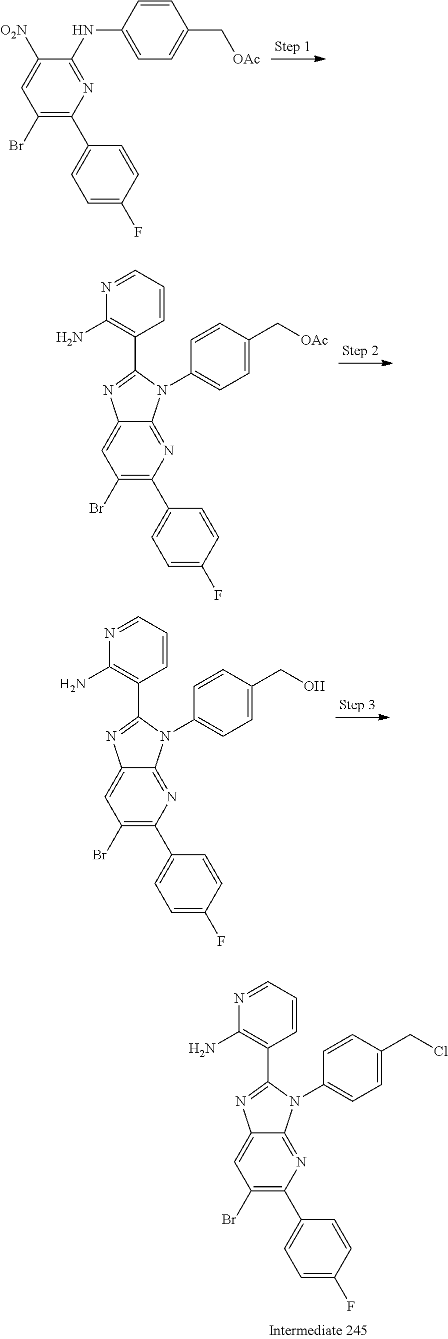

### Step 1: 4-(2-(2-Aminopyridin-3-yl)-6-bromo-5-(4-fluorophenyl)-3H-imidazo[4,5-b]pyridin-3-yl)benzyl acetate

To a solution of 4-((5-bromo-6-(4-fluorophenyl)-3-nitropyridin-2-yl)amino)benzyl acetate (refer to Intermediate 244 for detail procedures, 1 g, 2.17 mmol) in DMSO (10 mL) was added Na₂S₂O₄ (1.72 g, 8.69 mmol, 88% purity) and 2-aminonicotinaldehyde (318 mg, 2.61 mmol). The mixture was stirred at 100° C. for 4 hr. The reaction mixture was quenched with H₂O (50 mL) at 25° C., diluted with CH₂Cl₂ (50 mL), and extracted with CH₂Cl₂ (25 mL×2). The combined organic layers were washed with brine (100 mL×3), dried over Na₂SO₄, filtered, and concentrated under reduced pressure. After purified by silica gel flash chromatography (Eluent of 30%˜50% EtOAc in petroleum ether), 4-(2-(2-aminopyridin-3-yl)-6-bromo-5-(4-fluorophenyl)-3H-imidazo[4,5-b]pyridin-3-yl)benzyl acetate (280 mg, yield: 24%) was obtained as a yellow solid. MS: m/z=531.9, 533.9 [M+H]+. ¹H NMR (400 MHz, Dimethylsulfoxide-d₆) δ 8.65 (s, 1H), 8.02 (dd, J=4.8, 1.6 Hz, 1H), 7.60 (dd, J=8.4, 5.6 Hz, 2H), 7.52-7.45 (m, 4H), 7.32-7.27 (m, 2H), 7.24 (dd, J=7.6, 1.6 Hz, 1H), 6.86 (br s, 2H), 6.44 (dd, J=7.6, 5.2 Hz, 1H), 5.13 (s, 2H), 2.09 (s, 3H).

### Step 2: (4-(2-(2-Aminopyridin-3-yl)-6-bromo-5-(4-fluorophenyl)-3H-imidazo[4,5-b]pyridin-3-yl)phenyl)methanol

To a solution of 4-(2-(2-aminopyridin-3-yl)-6-bromo-5-(4-fluorophenyl)-3H-imidazo[4,5-b]pyridin-3-yl)benzyl acetate (240 mg, 451 μmol) in THF (2 mL), MeOH (2 mL) and H₂O (2 mL) was added K₂CO₃ (187 mg, 1.35 mmol). The mixture was stirred at 25° C. for 1 hr. The reaction mixture was quenched with H₂O (20 mL) at 25° C., diluted with CH₂Cl₂ (20 mL) and extracted with CH₂Cl₂ (25 mL×2). The combined organic layers were washed with brine (50 mL), dried over Na₂SO₄, filtered and concentrated under reduced pressure. (4-(2-(2-Aminopyridin-3-yl)-6-bromo-5-(4-fluorophenyl)-3H-imidazo[4,5-b]pyridin-3-yl)phenyl)methanol (240 mg) was obtained as a yellow solid. MS: m/z=490.0, 492.0 [M+H]⁺. ¹H NMR (400 MHz, Dimethylsulfoxide-d₆) δ 8.63 (s, 1H), 8.00 (dd, J=4.8, 1.6 Hz, 1H), 7.62-7.57 (m, 2H), 7.44-7.38 (m, 4H), 7.28-7.26 (m, 2H), 7.21 (dd, J=7.6, 2.0 Hz, 1H), 6.93 (br s, 2H), 6.42 (dd, J=7.6, 4.4 Hz, 1H), 5.32 (t, J=5.2 Hz, 1H), 4.55 (d, J=5.2 Hz, 2H).

### Step 3: 3-(6-Bromo-3-(4-(chloromethyl)phenyl)-5-(4-fluorophenyl)-3H-imidazo[4,5-b]pyridin-2-yl)pyridin-2-amine

To a solution of (4-(2-(2-aminopyridin-3-yl)-6-bromo-5-(4-fluorophenyl)-3H-imidazo[4,5-b]pyridin-3-yl)phenyl)methanol (240 mg, 489 μmol) in CH₂Cl₂ (10 mL) was added SOCl₂ (175 mg, 1.47 mmol) at 0° C. The mixture was stirred at 40° C. for 0.3 hr. The reaction was concentrated under reduced pressure to give 3-(6-bromo-3-(4-(chloromethyl)phenyl)-5-(4-fluorophenyl)-3H-imidazo[4,5-b]pyridin-2-yl)pyridin-2-amine (Intermediate 245, 266 mg, HCl salt) as a yellow solid. MS: m/z=508.0, 509.8 [M+H]⁺.

### Intermediate 246: 2-(2-Aminopyridin-3-yl)-1-(4-(chloromethyl)phenyl)-1H-imidazo[4,5-c]pyridine-6-carbonitrile

### Step 1: (4-((2-Bromo-5-nitropyridin-4-yl)amino)phenyl)methanol

To a solution of 2,4-dibromo-5-nitropyridine (4 g, 14.2 mmol) in DMSO (50 mL) were added DIEA (5.50 g, 42.6 mmol) and (4-aminophenyl)methanol (1.75 g, 14.2 mmol). The mixture was stirred at 100° C. for 16 hr. The reaction mixture was quenched with H₂O (500 mL) at 25° C., diluted with EtOAc (500 mL), and extracted with EtOAc (500 mL×2). The combined organic layers were washed with brine (500 mL×3), dried over Na₂SO₄, filtered and concentrated under reduced pressure to give (4-((2-bromo-5-nitropyridin-4-yl)amino)phenyl)methanol (4.6 g, yield: 88%) as a light yellow solid. MS: m/z=323.9, 325.9 [M+H]⁺. ¹H NMR (400 MHz, Chloroform-d) δ 9.80 (s, 1H), 9.22 (s, 1H), 7.70 (d, J=8.0 Hz, 2H), 7.45 (d, J=8.4 Hz, 3H), 4.96 (s, 2H).

### Step 2: 4-((2-Bromo-5-nitropyridin-4-yl)amino)benzyl acetate

To a solution of (4-((2-bromo-5-nitropyridin-4-yl)amino)phenyl)methanol (4.5 g, 13.9 mmol) in CH₂Cl₂ (50 mL) were added DMAP (170 mg, 1.39 mmol), Ac₂O (1.42 g, 13.9 mmol) and TEA (4.21 g, 41.7 mmol). The mixture was stirred at 25° C. for 1 hr. The reaction mixture was quenched with H₂O (100 mL) at 25° C., diluted with CH₂Cl₂ (50 mL), and extracted with CH₂Cl₂ (50 mL×2). The combined organic layers were washed with brine (50 mL), dried over Na₂SO₄, filtered, and concentrated under reduced pressure. After purified by silica gel flash chromatography (Eluent of 0%˜19% EtOAc in petroleum ether), 4-((2-bromo-5-nitropyridin-4-yl)amino)benzyl acetate (1.5 g, yield: 29%) was obtained as a yellow solid. MS: m/z=365.9, 367.9 [M+H]⁺. ¹H NMR (400 MHz, Chloroform-d) δ 9.62 (s, 1H), 9.03 (s, 1H), 7.50 (d, J=8.4 Hz, 2H), 7.28 (d, J=8.4 Hz, 2H), 7.10 (s, 1H), 5.15 (s, 2H), 2.14 (s, 3H).

### Step 3: 4-((2-Cyano-5-nitropyridin-4-yl)amino)benzyl acetate

A mixture of 4-((2-bromo-5-nitropyridin-4-yl)amino)benzyl acetate (1.5 g, 4.10 mmol), dicyanozinc (2.28 g, 19.4 mmol), Pd(PPh₃)₄ (473 mg, 410 μmol) in DMF (15 mL) was degassed and purged with N₂ three times, then stirred at 80° C. for 3 hr under N₂ atmosphere. The reaction mixture was quenched with H₂O (500 mL) at 25° C. and extracted with CH₂Cl₂ (500 mL×3). The combined organic layers were washed with brine (100 mL×3), dried over Na₂SO₄, filtered, and concentrated under reduced pressure. After purified by silica gel flash chromatography (Eluent of 5%˜15% EtOAc in petroleum ether), 4-((2-cyano-5-nitropyridin-4-yl)amino)benzyl acetate (1.1 g, yield: 86%) was obtained as a light yellow solid. MS: m/z=312.9 [M+H]⁺.

### Step 4: 4-(2-(2-Aminopyridin-3-yl)-6-cyano-1H-imidazo[4,5-c]pyridin-1-yl)benzyl acetate

To a solution of 4-((2-cyano-5-nitropyridin-4-yl)amino)benzyl acetate (1 g, 3.20 mmol) in DMSO (20 mL) were added Na₂S₂O₄ (2.23 g, 12.8 mmol, 88% purity) and 2-aminonicotinaldehyde (469 mg, 3.84 mmol). The mixture was stirred at 100° C. for 16 hr. The reaction mixture was quenched with H₂O (300 mL) at 25° C., diluted with CH₂C12 (300 mL) and extracted with CH₂Cl₂ (300 mL×2). The combined organic layers were washed with brine (500 mL), dried over Na₂SO₄, filtered and concentrated under reduced pressure. After purified by silica gel flash chromatography (Eluent of 0%˜27% EtOAc in petroleum ether), 4-(2-(2-aminopyridin-3-yl)-6-cyano-1H-imidazo[4,5-c]pyridin-1-yl)benzyl acetate (160 mg, yield: 11%) was obtained as a light yellow solid. MS: m/z=385.1 [M+H]⁺.

### Step 5: 2-(2-Aminopyridin-3-yl)-1-(4-(hydroxymethyl)phenyl)-1H-imidazo[4,5-c]pyridine-6-carbonitrile

To a solution of 4-(2-(2-aminopyridin-3-yl)-6-cyano-1H-imidazo[4,5-c]pyridin-1-yl)benzyl acetate (160 mg, 416 μmol) in THF (2 mL), MeOH (2 mL) and H₂O (1 mL) was added K₂CO₃ (173 mg, 1.25 mmol). The mixture was stirred at 25° C. for 0.5 hr. The reaction mixture was diluted with H₂O (50 mL) and extracted with CH₂Cl₂ (50 mL×2). The combined organic layers were washed with brine (100 mL), dried over Na₂SO₄, filtered and concentrated under reduced pressure. 2-(2-Aminopyridin-3-yl)-1-(4-(hydroxymethyl)phenyl)-1H-imidazo[4,5-c]pyridine-6-carbonitrile (160 mg crude) was obtained as a light yellow solid. MS: m/z=343.1 [M+H]+.

### Step 6: 2-(2-Aminopyridin-3-yl)-1-(4-(chloromethyl)phenyl)-1H-imidazo[4,5-c]pyridine-6-carbonitrile

To a solution of 2-(2-aminopyridin-3-yl)-1-(4-(hydroxymethyl)phenyl)-1H-imidazo[4,5-c]pyridine-6-carbonitrile (160 mg, 467 μmol) in CH₂Cl₂ (2 mL) was added SOCl₂ (167 mg, 1.40 mmol). The mixture was stirred at 40° C. for 0.5 hr. The reaction was concentrated under reduced pressure to give 2-(2-aminopyridin-3-yl)-1-(4-(chloromethyl)phenyl)-1H-imidazo[4,5-c]pyridine-6-carbonitrile (Intermediate 246, 160 mg, HCl salt) as a light yellow solid. MS: m/z=361.0 [M+H]⁺.

### Intermediate 247: 3-(3-(4-(Chloromethyl)phenyl)-5-(1-methyl-1H-1,2,4-triazol-3-yl)-3H-imidazo[4,5-b]pyridin-2-yl)pyridin-2-amine

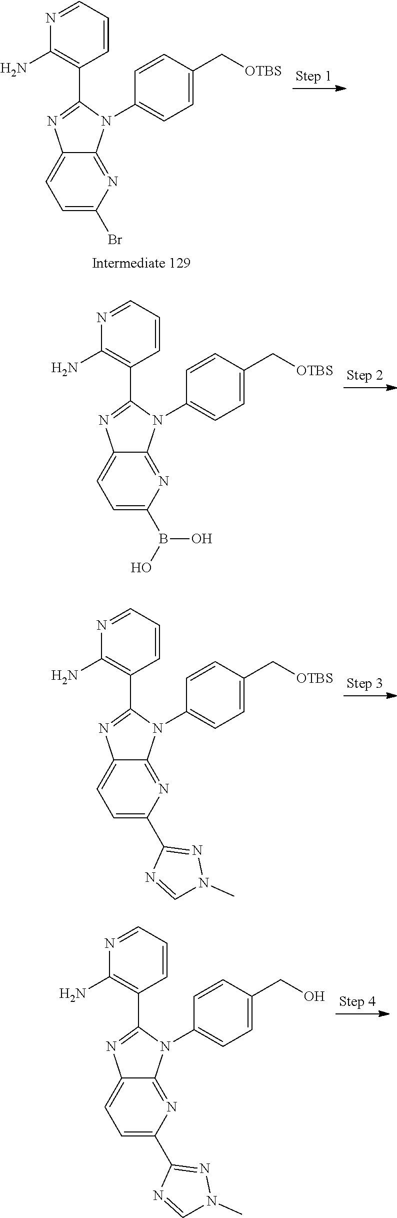

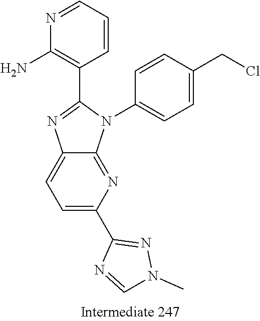 [SMILES: Cn1cnc(-c2ccc3nc(-c4cccnc4N)n(-c4ccc(CCl)cc4)c3n2)n1]

### Step 1; (2-(2-Aminopyridin-3-yl)-3-(4-(((tert-butyldimethylsilyl)oxy)methyl)phenyl)-3H-imidazo[4,5-b]pyridin-5-yl)boronic acid

A mixture of Intermediate 129 (1 g, 1.96 mmol), 4,4,4′,4′,5,5,5′,5′-octamethyl-2,2′-bi(1,3,2-dioxaborolane) (995 mg, 3.92 mmol), KOAc (577 mg, 5.88 mmol) and Pd(dppf)Cl₂ (143 mg, 196 μmol) in 1,4-dioxane (20 mL) was degassed and purged with N₂ three times. The mixture was stirred at 80° C. for 2 hr under N₂ atmosphere. The mixture was used in the next step without work-up and purification. (2-(2-Aminopyridin-3-yl)-3-(4-(((tert-butyldimethylsilyl)oxy)methyl)phenyl)-3H-imidazo[4,5-b]pyridin-5-yl)boronic acid was obtained as a black solution. MS: m/z=476.2 [M+H]⁺.

### Step 2: 3-(3-(4-(((tert-Butyldimethylsilyl)oxy)methyl)phenyl)-5-(1-methyl-1H-1,2,4-triazol-3-yl)-3H-imidazo[4,5-b]pyridin-2-yl)pyridin-2-amine

To a solution of (2-(2-aminopyridin-3-yl)-3-(4-(((tert-butyldimethylsilyl)oxy)methyl)phenyl)-3H-imidazo[4,5-b]pyridin-5-yl)boronic acid in H₂O (2 mL) and 1,4-dioxane (10 mL) were added 3-bromo-1-methyl-1H-1,2,4-triazole (348 mg, 2.15 mmol), Pd(dppf)Cl₂ (131 mg, 179 μmol) and Cs₂CO₃ (1.75 g, 5.36 mmol). The mixture was degassed and purged with N₂ three times and stirred at 80° C. for 16 hr under N₂ atmosphere. The reaction mixture was quenched with H₂O (50 mL) and extracted with CH₂Cl₂ (50 mL×2). The combined organic layers were washed with brine (50 mL), dried over Na₂SO₄, filtered, and concentrated under reduced pressure. After purified by silica gel flash chromatography (Eluent of 20%˜80% EtOAc in petroleum ether), 3-(3-(4-(((tert-butyldimethylsilyl)oxy)methyl)phenyl)-5-(I-methyl-1H-1,2,4-triazol-3-yl)-3H-imidazo[4,5-b]pyridin-2-yl)pyridin-2-amine (310 mg, yield: 31% for two steps) was obtained as a black solid. MS: m/z=513.2 [M+H]⁺. ¹H NMR (400 MHz, Dimethylsulfoxide-d₆) δ 8.52 (s, 1H), 8.27 (d, J=8.4 Hz, 1H), 8.11 (d, J=8.4 Hz, 1H), 8.01-7.97 (m, 1H), 7.52-7.42 (m, 4H), 7.18 (dd, J=7.6, 1.6 Hz, 1H), 6.99 (s, 2H), 6.37 (dd, J=7.6, 4.8 Hz, 1H), 4.83 (s, 2H), 3.92 (s, 3H), 0.94 (s, 9H), 0.13 (s, 6H).

### Step 3: (4-(2-(2-Aminopyridin-3-yl)-5-(1-methyl-1H-1,2,4-triazol-3-yl)-3H-imidazo[4,5-b]pyridin-3-yl)phenyl)methanol

To a solution of 3-(3-(4-(((tert-butyldimethylsilyl)oxy)methyl)phenyl)-5-(1-methyl-1H-1,2,4-triazol-3-yl)-3H-imidazo[4,5-b]pyridin-2-yl)pyridin-2-amine (310 mg, 605 μmol) in THF (5 mL) was added TBAF (1.21 mL, 1 M in THF). The mixture was stirred at 25° C. for 0.5 hr. The reaction mixture was quenched with H₂O (20 mL) at 25° C. and extracted with CH₂Cl₂ (20 mL×3). The combined organic layers were washed with brine (20 mL×2), dried over Na₂SO₄, filtered and concentrated under reduced pressure. (4-(2-(2-Aminopyridin-3-yl)-5-(1-methyl-1H-1,2,4-triazol-3-yl)-3H-imidazo[4,5-b]pyridin-3-yl)phenyl)methanol (150 mg, yield: 62%) was obtained as a black solid. MS: m/z=399.1 [M+H]⁺. ¹H NMR (400 MHz, Dimethylsulfoxide-d₆) δ 8.52 (s, 1H), 8.26 (d, J=8.4 Hz, 1H), 8.10 (d, 0.1=8.4 Hz, 1H), 8.00-7.97 (m, 1H), 7.50-7.41 (m, 4H), 7.24-7.20 (m, 1H), 6.96 (s, 2H), 6.40 (dd, J=7.6, 5.2 Hz, 1H), 5.39-5.33 (m, 1H), 4.60 (d, J=6.0 Hz, 2H), 3.92 (s, 3H).

### Step 4: 3-(3-(4-(Chloromethyl)phenyl)-5-(1-methyl-1H-1,2,4-triazol-3-yl)-3H-imidazo[4,5-b]pyridin-2-yl)pyridin-2-amine

To a solution of (4-(2-(2-aminopyridin-3-yl)-5-(1-methyl-1H-1,2,4-triazol-3-yl)-3H-imidazo[4,5-b]pyridin-3-yl)phenyl)methanol (160 mg, 376 μmol) in CH₂Cl₂ (5 mL) was added SOCl₂ (135 mg, 1.13 mmol). The mixture was stirred at 25° C. for 0.5 hr. The reaction was concentrated under reduced pressure to give 3-(3-(4-(chloromethyl)phenyl)-5-(1-methyl-1H-1,2,4-triazol-3-yl)-3H-imidazo[4,5-b]pyridin-2-yl)pyridin-2-amine (Intermediate 247, 156 mg) as a brown solid. MS: m/z=417.1 [M+H]⁺.

### Intermediate 248: 2-(2-Aminopyridin-3-yl)-3-(4-(chloromethyl)phenyl)-5-methyl-3H-imidazo[4,5-b]pyridine-6-carbonitrile

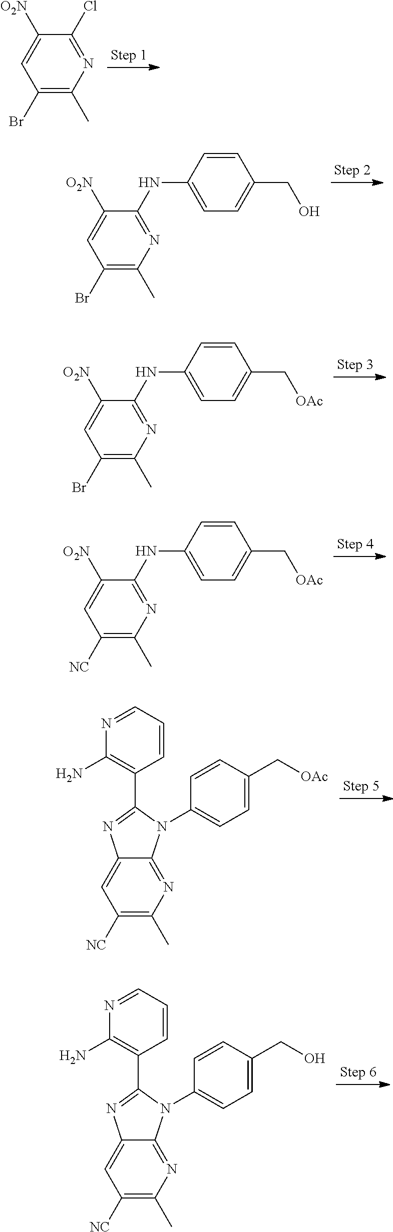

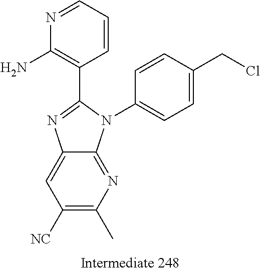 [SMILES: Cc1nc2c(cc1C#N)nc(-c1cccnc1N)n2-c1ccc(CCl)cc1]

### Step 1: (4-((5-Bromo-6-methyl-3-nitropyridin-2-yl)amino)phenyl)methanol

To a solution of 3-bromo-6-chloro-2-methyl-5-nitropyridine (1 g, 3.98 mmol) in DMSO (10 mL) were added DIEA (1.54 g, 11.9 mmol) and (4-aminophenyl)methanol (490 mg, 3.98 mmol). The mixture was stirred at 100° C. for 16 hr. The reaction mixture was quenched with H₂O (50 mL) at 25° C., diluted with CH₂Cl₂ (50 mL), and extracted with CH₂Cl₂ (50 mL×2). The combined organic layers were washed with brine (50 mL×3), dried over Na₂SO₄, filtered and concentrated under reduced pressure. (4-((5-Bromo-6-methyl-3-nitropyridin-2-yl)amino)phenyl)methanol (1.3 g) was obtained as a black solid. MS: m/z=337.8, 339.8 [M+H]⁺. ¹H NMR (400 MHz, Dimethylsulfoxide-d₆) δ 9.96 (s, 1H), 8.60 (s, 1H), 7.60 (d, J=8.4 Hz, 2H), 7.31 (d, 1=8.4 Hz, 2H), 5.16 (t, J=6.0 Hz, 1H), 4.48 (d, 0.1=5.2 Hz, 2H), 2.52 (s, 3H).

### Step 2: 4-((5-Bromo-6-methyl-3-nitropyridin-2-yl)amino)benzyl acetate

To a solution of (4-((5-bromo-6-methyl-3-nitropyridin-2-yl)amino)phenyl)methanol (1.3 g, 3.84 mmol) in CH₂Cl₂ (15 mL) were added DMAP (93.9 mg, 769 μmol), Ac₂O (589 mg, 5.77 mmol) and TEA (1.17 g, 11.5 mmol). The mixture was stirred at 25° C. for 1 hr. The reaction mixture was quenched with H₂O (25 mL) at 25° C., diluted with CH₂Cl₂ (50 mL) and extracted with CH₂Cl₂ (50 mL×2). The combined organic layers were washed with brine (50 mL), dried over Na₂SO₄, filtered, and concentrated under reduced pressure. After purified by silica gel flash chromatography (Eluent of 1%˜10% EtOAc in petroleum ether), 4-((5-bromo-6-methyl-3-nitropyridin-2-yl)amino)benzyl acetate (850 mg, yield: 58% for two steps) was obtained as a red solid. MS: m/z=377.8, 379.8 [M+H]⁺. ¹H NMR (400 MHz, Dimethylsulfoxide-d₆) δ 9.98 (s, 1H), 8.62 (s, 1H), 7.68 (d, J=8.4 Hz, 2H), 7.36 (d, J=8.4 Hz, 2H), 5.05 (s, 2H), 2.54 (s, 3H), 2.07 (s, 3H).

### Step 3: 4-((5-Cyano-6-methyl-3-nitropyridin-2-yl)amino)benzyl acetate

A mixture of 4-((5-bromo-6-methyl-3-nitropyridin-2-yl)amino)benzyl acetate (1 g, 2.63 mmol) and CuCN (471 mg, 5.26 mmol) in DMF (10 mL) was degassed and purged with N₂ three times. The mixture was stirred at 140° C. for 3 hr under N₂ atmosphere. The reaction mixture was quenched with H₂O (50 mL) at 25° C. and extracted with EtOAc (50 mL×3). The combined organic layers were washed with brine (100 mL×3), dried over Na₂SO₄, filtered and concentrated under reduced pressure. After purified by silica gel flash chromatography (Eluent of 5%˜15% EtOAc in petroleum ether), 4-((5-cyano-6-methyl-3-nitropyridin-2-yl)amino)benzyl acetate (480 mg, yield: 56%) was obtained as a yellow solid. MS: m/z=326.9 [M+H]⁺. ¹H NMR (400 MHz, Dimethylsulfoxide-d₆) δ 10.32 (s, 1H), 8.94 (s, 1H), 7.65 (d, J=8.8 Hz, 2H), 7.39 (d, J=8.4 Hz, 2H), 5.08 (s, 2H), 2.56 (s, 3H), 2.07 (s, 3H).

### Step 4: 4-(2-(2-Aminopyridin-3-yl)-6-cyano-5-methyl-3H-imidazo[4,5-b]pyridin-3-yl)benzyl acetate

To a solution of 4-((5-cyano-6-methyl-3-nitropyridin-2-yl)amino)benzyl acetate (700 mg, 2.15 mmol) in DMSO (1 mL) were added Na₂S₂O₄ (1.70 g, 8.58 mmol, 88% purity) and 2-aminonicotinaldehyde (314 mg, 2.57 mmol). The mixture was stirred at 100° C. for 6 hr. The reaction mixture was quenched with H₂O (30 mL) at 25° C., diluted with CH₂Cl₂ (30 mL) and extracted with CH₂Cl₂ (50 mL×2). The combined organic layers were washed with brine (50 mL), dried over Na₂SO₄, filtered and concentrated under reduced pressure. After purified by silica gel flash chromatography (Eluent of 20%˜50% EtOAc in petroleum ether), 4-(2-(2-aminopyridin-3-yl)-6-cyano-5-methyl-3H-imidazo[4,5-b]pyridin-3-yl)benzyl acetate (200 mg, yield: 23%) was obtained as a light yellow solid. MS: m/z=399.0 [M+H]⁺. ¹H NMR (400 MHz, Dimethylsulfoxide-d₆) δ 8.72 (s, 1H), 8.00 (dd, J=4.8, 2.0 Hz, 1H), 7.54-7.50 (m, 2H), 7.48-7.43 (m, 2H), 7.25 (dd, J=7.6, 1.6 Hz, 1H), 6.82 (br s, 2H), 6.43 (dd, J=7.6, 4.8 Hz, 1H), 5.15 (s, 2H), 2.70 (s, 3H), 2.11 (s, 3H).

### Step 5: 2-(2-Aminopyridin-3-yl)-3-(4-(hydroxymethyl)phenyl)-5-methyl-3H-imidazo[4,5-b]pyridine-6-carbonitrile

To a solution of 4-(2-(2-aminopyridin-3-yl)-6-cyano-5-methyl-3H-imidazo[4,5-b]pyridin-3-yl)benzyl acetate (180 mg, 452 μmol) in THF (2 mL) and MeOH (2 mL) was added K₂CO₃ (187 mg, 1.36 mmol). The mixture was stirred at 25° C. for 1 hr. The reaction mixture was diluted with H₂O (30 mL) and extracted with CH₂Cl₂ (50 mL×2). The combined organic layers were washed with brine (100 mL), dried over Na₂SO₄, filtered and concentrated under reduced pressure. 2-(2-Aminopyridin-3-yl)-3-(4-(hydroxymethyl)phenyl)-5-methyl-3H-imidazo[4,5-b]pyridine-6-carbonitrile (170 mg) was obtained as a yellow solid. MS: m/z=357.0 [M+H]⁺. ¹H NMR (400 MHz, Dimethylsulfoxide-d₆) δ 8.71 (s, 1H), 8.00 (dd, J=4.8, 1.6 Hz, 1H), 7.48-7.44 (m, 2H), 7.42-7.37 (m, 2H), 7.24 (dd, J=7.6, 1.6 Hz, 1H), 6.90 (br s, 2H), 6.41 (dd, J=7.6, 4.8 Hz, 1H), 5.35 (t, J=5.6 Hz, 1H), 4.58 (d, J=5.6 Hz, 2H), 2.69 (s, 3H).

### Step 6: 2-(2-Aminopyridin-3-yl)-3-(4-(chloromethyl)phenyl)-5-methyl-3H-imidazo[4,5-b]pyridine-6-carbonitrile

To a solution of 2-(2-aminopyridin-3-yl)-3-(4-(hydroxymethyl)phenyl)-5-methyl-3H-imidazo[4,5-b]pyridine-6-carbonitrile (170 mg, 477 μmol) in CH₂Cl₂ (5 mL) was added SOCl₂ (170 mg, 1.43 mmol). The mixture was stirred at 25° C. for 1 hr. The reaction was concentrated under reduced pressure, 2-(2-aminopyridin-3-yl)-3-(4-(chloromethyl)phenyl)-5-methyl-3H-imidazo[4,5-b]pyridine-6-carbonitrile (Intermediate 248, 196 mg, HCl salt) was obtained as a yellow solid. MS: m/z=375.0 [M+H]+.

### Intermediate 249 & 250: 4-((6-(3-Methyl-4H-1,2,4-triazol-4-yl)-3-nitropyridin-2-yl)amino)benzyl acetate & 4-((6-(3-Methyl-1H-1,2,4-triazol-1-yl)-3-nitropyridin-2-yl)amino)benzyl acetate

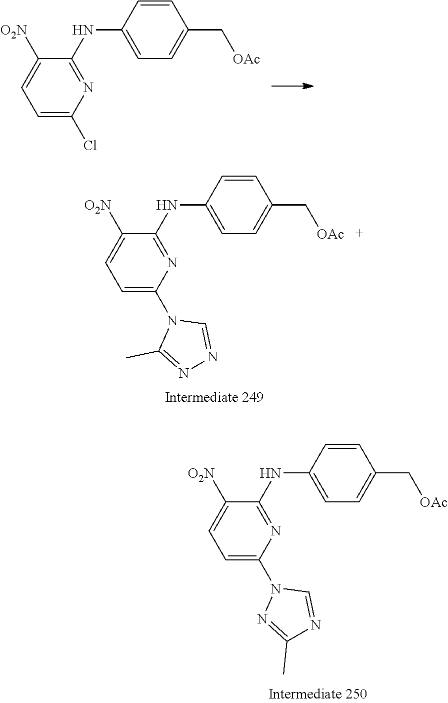

To a solution of 4-((6-chloro-3-nitropyridin-2-yl)amino)benzyl acetate (Refer to Intermediate 178 for detail procedures, 3 g, 9.33 mmol) and 3-methyl-4H-1,2,4-triazole (929 mg, 11.19 mmol) in DMSO (30 mL) was added DLEA (4.87 mL). The mixture was stirred at 100° C. for 16 hr. The reaction mixture was diluted with H₂O (100 mL) and extracted with CH₂Cl₂ (200 mL). The combined organic layers were washed with brine (100 mL), dried over anhydrous Na₂SO₄, filtered and concentrated under reduced pressure. After purified by prep-HPLC (column: Phenomenex luna C18 (250×70 mm, 10 μm); mobile phase: [water (HCl)-ACN]; gradient: 32%-72% B over 28 min), 4-((6-(3-methyl-4H-1,2,4-triazol-4-yl)-3-nitropyridin-2-yl)amino)benzyl acetate (Intermediate 249, 475 mg, yield: 14%) was obtained as a yellow solid. MS: m/z=369.1 [M+H]+ 1H NMR (400 MHz, Dimethylsulfoxide-d₆) δ 10.20 (br s, 1H), 8.72 (d, J=8.8 Hz, 1H), 8.11 (s, 1H), 7.49 (d, J=8.4 Hz, 2H), 7.41 (d, J=8.4 Hz, 2H), 7.34 (d, J=9.2 Hz, 1H), 5.09 (s, 2H), 2.27 (s, 3H), 2.08 (s, 3H). 4-((6-(3-Methyl-1H-1,2,4-triazol-1-yl)-3-nitropyridin-2-yl)amino)benzyl acetate (Intermediate 250, 1.1 g, yield: 32%) was obtained as a yellow solid. MS: m/z=369.2 [M+H]^{+ 1}H NMR (400 MHz, Dimethylsulfoxide-d₆) δ 10.25 (br s, 1H), 8.88 (s, 1H), 8.75 (d, J=8.4, 1H), 7.71 (d, J=7.2 Hz, 2H), 7.44 (d, J=7.2 Hz, 2H), 7.30 (d, J=8.4 Hz, 1H), 5.09 (s, 2H), 2.27 (s, 3H), 2.08 (s, 3H).

### Intermediate 251: 3-(3-(4-(Chloromethyl)phenyl)-5-(3-methyl-4H-1,2,4-triazol-4-yl)-3H-imidazo[4,5-b]pyridin-2-yl)pyridin-2-amine

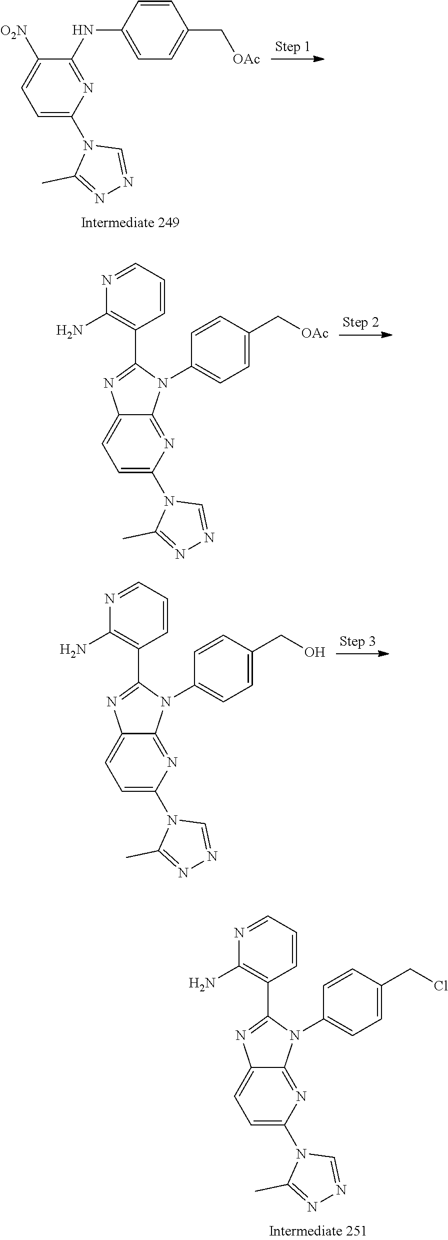

### Step 1: 4-(2-(2-Aminopyridin-3-yl)-5-(3-methyl-4H-1,2,4-triazol-4-yl)-3H-imidazo[4,5-b]pyridin-3-yl)benzyl acetate

To a solution of Intermediate 249 (400 mg, 1.09 mmol), 2-aminonicotinaldehyde (146 mg, 1.19 mmol) in DMSO (20 mL) was added Na₂S₂O₄ (869 mg, 4.34 mmol). The mixture was stirred at 100° C. for 16 hr. The reaction mixture was poured into H₂O (20 mL), extracted with EtOAc (15 mL×3). The combined organic phases were dried over anhydrous Na₂SO₄, filtered and concentrated under reduced pressure. After purified by silica gel flash chromatography (Eluent of 0%˜69% EtOAc in petroleum ether), 4-(2-(2-aminopyridin-3-yl)-5-(3-methyl-4H-1,2,4-triazol-4-yl)-3H-imidazo[4,5-b]pyridin-3-yl)benzyl acetate (150 mg, yield: 31%) was obtained as a gray solid. MS: m/z=441.3 [M+H]⁺.

### Step 2: (4-(2-(2-Aminopyridin-3-yl)-5-(3-methyl-4H-1,2,4-triazol-4-yl)-3H-imidazo[4,5-b]pyridin-3-yl)phenyl)methanol

To mixture of 4-(2-(2-aminopyridin-3-yl)-5-(3-methyl-4H-1,2,4-triazol-4-yl)-3H-imidazo[4,5-b]pyridin-3-yl)benzyl acetate (150 mg, 341 μmol) in THF (1.5 mL) and MeOH (1.5 mL) was added K₂CO₃ (94.1 mg, 681 μmol) in H₂O (1 mL). The mixture was stirred at 25° C. for 0.5 hr. The reaction mixture was concentrated under reduced pressure. The residue was diluted with H₂O (5 mL) and extracted with EtOAc (10 mL×3). The organic phase was dried over anhydrous Na₂SO₄, filtered and concentrated under reduced pressure. (4-(2-(2-Aminopyridin-3-yl)-5-(3-methyl-4H-1,2,4-triazol-4-yl)-3H-imidazo[4,5-b]pyridin-3-yl)phenyl)methanol (145 mg) was obtained as a gray solid. MS: m/z=399.2 [M+H]⁺.

### Step 3: 3-(3-(4-(Chloromethyl)phenyl)-5-(3-methyl-4H-1,2,4-triazol-4-yl)-3H-imidazo[4,5-b]pyridin-2-yl)pyridin-2-amine

To a solution of (4-(2-(2-aminopyridin-3-yl)-5-(3-methyl-4H-1,2,4-triazol-4-yl)-3H-imidazo[4,5-b]pyridin-3-yl)phenyl)methanol (145 mg, 364 μmol) in CH₂Cl₂ (2 mL) was added SOCl₂ (1 mL). The mixture was stirred at 25° C. for 0.5 hr. The reaction mixture was concentrated under reduced pressure. 3-(3-(4-(Chloromethyl)phenyl)-5-(3-methyl-4H-1,2,4-triazol-4-yl)-3H-imidazo[4,5-b]pyridin-2-yl)pyridin-2-amine (Intermediate 251, 166 mg) was obtained as a gray solid. MS: m/z=417.1, 419.1 [M+H]⁺.

### Intermediate 252: 3-(3-(4-(Chloromethyl)phenyl)-5-(3-methyl-1H-1,2,4-triazol-1-yl)-3H-imidazo[4,5-b]pyridin-2-yl)pyridin-2-amine

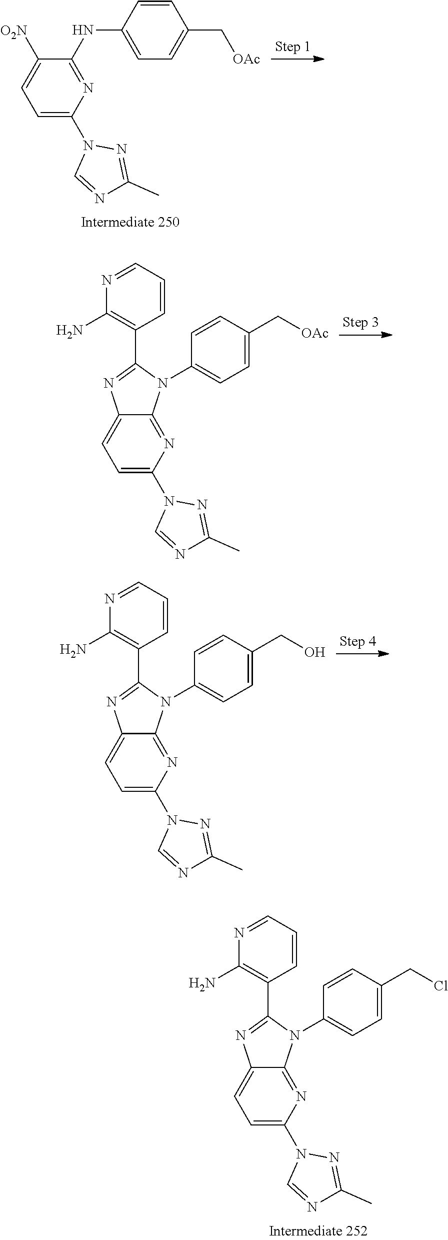

### Step 1: 4-(2-(2-Aminopyridin-3-yl)-5-(3-methyl-1H-1,2,4-triazol-1-yl)-3H-imidazo[4,5-b]pyridin-3-yl)benzyl acetate

To a solution of Intermediate 250 (550 mg, 1.49 mmol) in DMSO (5 mL) were added Na₂S₂O₄ (1.04 g, 5.97 mmol, 88% purity) and 2-aminonicotinaldehyde (219 mg, 1.79 mmol). The mixture was stirred at 100° C. for 6 hr. The reaction mixture was quenched with H₂O (50 mL) at 25° C., diluted with EtOAc (50 mL), and extracted with EtOAc (50 mL×2). The combined organic layers were washed with brine (50 mL), dried over Na₂SO₄, filtered and concentrated under reduced pressure. After purified by silica gel flash chromatography (Eluent of 0%˜63% EtOAc in petroleum ether), 4-(2-(2-aminopyridin-3-yl)-5-(3-methyl-1H-1,2,4-triazol-1-yl)-3H-imidazo[4,5-b]pyridin-3-yl)benzyl acetate (200 mg, yield: 24%) was obtained as a light yellow solid. MS: m/z=441.1 [M+H]⁺. ¹H NMR (400 MHz, Chloroform-d) δ 8.86 (s, 1H), 8.22 (d, J=8.8 Hz, 1H), 8.09 (d, J=3.2 Hz, 1H), 7.91 (d, J=8.4 Hz, 1H), 7.54 (d, J=8.0 Hz, 2H), 7.40 (d, J=8.4 Hz, 2H), 7.09 (br d, J=7.6 Hz, 1H), 6.61 (s, 2H), 6.40 (dd, J=7.6, 4.8 Hz, 1H), 5.24 (s, 2H), 2.50 (s, 3H), 2.18 (s, 3H)

### Step 2: (4-(2-(2-Aminopyridin-3-yl)-5-(3-methyl-1H-1,2,4-triazol-1-yl)-3H-imidazo[4,5-b]pyridin-3-yl)phenyl)methanol

To a solution of 4-(2-(2-aminopyridin-3-yl)-5-(3-methyl-1H-1,2,4-triazol-1-yl)-3H-imidazo[4,5-b]pyridin-3-yl)benzyl acetate (200 mg, 454 μmol) in THF (2 mL), MeOH (2 mL) and H₂O (1 mL) was added K₂CO₃ (188 mg, 1.36 mmol). The mixture was stirred at 25° C. for 1 hr. The reaction mixture was diluted with H₂O (50 mL) and extracted with EtOAc (50 mL×2). The combined organic layers were washed with brine (100 mL), dried over Na₂SO₄, filtered and concentrated under reduced pressure. (4-(2-(2-Aminopyridin-3-yl)-5-(3-methyl-1H-1,2,4-triazol-1-yl)-3H-imidazo[4,5-b]pyridin-3-yl)phenyl)methanol (150 mg) was obtained as a light yellow solid. MS: m/z=399.2 [M+H]⁺.

### Step 3: 3-(3-(4-(Chloromethyl)phenyl)-5-(3-methyl-1H-1,2,4-triazol-1-yl)-3H-imidazo[4,5-b]pyridin-2-yl)pyridin-2-amine

To a solution of (4-(2-(2-aminopyridin-3-yl)-5-(3-methyl-1H-1,2,4-triazol-1-yl)-3H-imidazo[4,5-b]pyridin-3-yl)phenyl)methanol (150 mg, 376 μmol) in CH₂Cl₂ (3 mL) was added SOCl₂ (0.1 mL). The mixture was stirred at 40° C. for 0.5 hr. The reaction was concentrated under reduced pressure to give 3-(3-(4-(chloromethyl)phenyl)-5-(3-methyl-1H-1,2,4-triazol-1-yl)-3H-imidazo[4,5-b]pyridin-2-yl)pyridin-2-amine (Intermediate 252, 150 mg, HCl salt) as a light yellow solid. MS: m/z=417.2, 419.2 [M+H]⁺.

### Intermediate 253: 3-(3-(4-(Chloromethyl)phenyl)-5-(1H-1,2,4-triazol-1-yl)-3H-imidazo[4,5-b]pyridin-2-yl)pyridin-2-amine

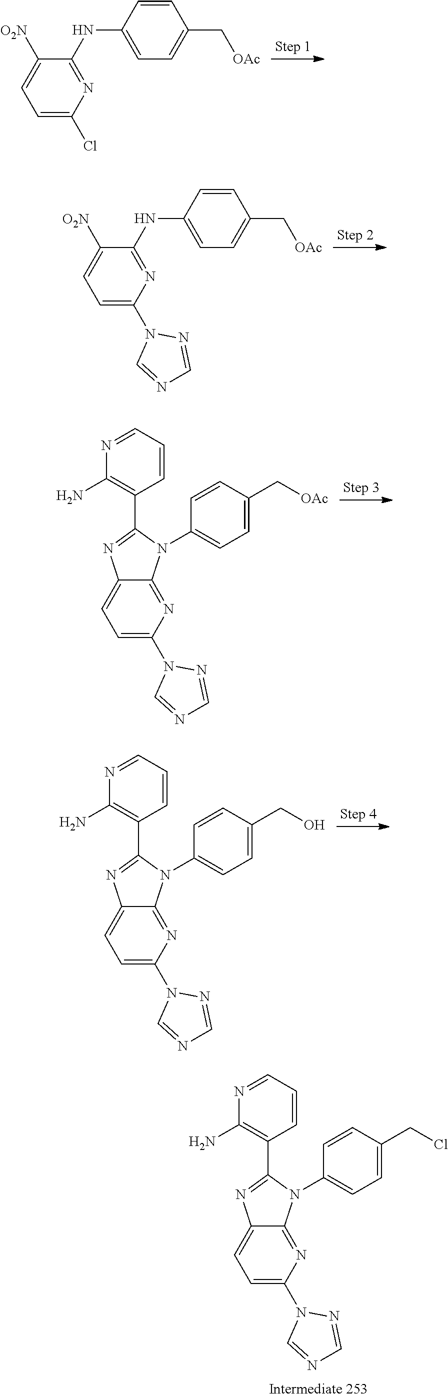 [SMILES: CC(=O)OCc1ccc(Nc2nc(Cl)ccc2[N+](=O)[O-])cc1 ; CC(=O)OCc1ccc(Nc2nc(-n3cncn3)ccc2[N+](=O)[O-])cc1 ; CC(=O)OCc1ccc(-n2c(-c3cccnc3N)nc3ccc(-n4cncn4)nc32)cc1 ; Nc1ncccc1-c1nc2ccc(-n3cncn3)nc2n1-c1ccc(CO)cc1 ; Nc1ncccc1-c1nc2ccc(-n3cncn3)nc2n1-c1ccc(CCl)cc1]

### Step 1: 4-((3-Nitro-6-(1H-1,2,4-triazol-1-yl)pyridin-2-yl)amino)benzyl acetate

To a solution of 4-((6-chloro-3-nitropyridin-2-yl)amino)benzyl acetate (refer to Intermediate 178 for detail procedures, 2 g, 6.22 mmol) and 4H-1,2,4-triazole (429 mg, 6.22 mmol) in DMF (30 mL) were added K₂CO₃ (4.30 g, 31.1 mmol) and NaI (93.2 mg, 622 μmol). The mixture was stirred at 80° C. for 1 hr. The reaction mixture was quenched with H₂O (500 mL) at 25° C., diluted with CH₂C12 (500 mL), and extracted with CH₂Cl₂ (500 mL×2). The combined organic layers were washed with brine (300 mL×3), dried over Na₂SO₄, filtered and concentrated under reduced pressure. After purified by silica gel flash chromatography (Eluent of 0%˜25% EtOAc in petroleum ether), 4-((3-nitro-6-(1H-1,2,4-triazol-1-yl)pyridin-2-yl)amino)benzyl acetate (1.1 g, yield: 48%) was obtained as a light yellow solid. MS: m/z=377.0 [M+Na]. ¹H NMR (400 MHz, Chloroform-d) δ 10.27 (s, 1H), 8.97 (s, 1H), 8.75 (d, J=8.8 Hz, 1H), 8.12 (s, 1H), 7.58 (d, J=8.4 Hz, 2H), 7.44 (dd, J=9.6, 8.8 Hz, 3H), 5.15 (s, 2H), 2.15 (s, 3H).

### Step 2: 4-(2-(2-Aminopyridin-3-yl)-5-(1H-1,2,4-triazol-1-yl)-3H-imidazo[4,5-b]pyridin-3-yl)benzyl acetate

To a solution of 4-((3-nitro-6-(1H-1,2,4-triazol-1-yl)pyridin-2-yl)amino)benzyl acetate (1 g, 2.82 mmol) in DMSO (10 mL) were added Na₂S₂O₄ (1.97 g, 11.3 mmol, 88% purity) and 2-aminonicotinaldehyde (414 mg, 3.39 mmol). The mixture was stirred at 100° C. for 6 hr. The reaction mixture was quenched with H₂O (50 mL) at 25° C., and then diluted with EtOAc (50 mL) and extracted with EtOAc (50 mL×2). The combined organic layers were washed with brine (50 mL), dried over Na₂SO₄, filtered and concentrated under reduced pressure. After purified by silica gel flash chromatography (Eluent of 0%˜53% EtOAc in petroleum ether), 4-(2-(2-aminopyridin-3-yl)-5-(1H-1,2,4-triazol-1-yl)-3H-imidazo[4,5-b]pyridin-3-yl)benzyl acetate (200 mg, yield: 16%) was obtained as a light yellow solid. MS: m/z=427.1 [M+H]⁺. ¹H NMR (400 MHz, Chloroform-d) δ 9.00 (s, 1H), 8.25 (d, J=8.4 Hz, 1H), 8.14-8.06 (m, 2H), 7.97 (d, J=8.4 Hz, 1H), 7.54 (d, J=8.4 Hz, 2H), 7.41 (d, J=8.4 Hz, 2H), 7.10 (d, J=7.6 Hz, 1H), 6.63 (s, 2H), 6.41 (dd, J=7.6, 4.8 Hz, 1H), 5.24 (s, 2H), 2.19 (s, 3H).

### Step 3: (4-(2-(2-Aminopyridin-3-yl)-5-(1H-1,2,4-triazol-1-yl)-3H-imidazo[4,5-b]pyridin-3-yl)phenyl)methanol

To a solution of 4-(2-(2-aminopyridin-3-yl)-5-(1H-1,2,4-triazol-1-yl)-3H-imidazo[4,5-b]pyridin-3-yl)benzyl acetate (200 mg, 469 μmol) in THF (2 mL), MeOH (2 mL) and H₂O (1 mL) was added K₂CO₃ (194 mg, 1.41 mmol). The mixture was stirred at 25° C. for 1 hr. The reaction mixture was diluted with H₂O (50 mL) and extracted with EtOAc (50 mL×2). The combined organic layers were washed with brine (100 mL), dried over Na₂SO₄, filtered and concentrated under reduced pressure. (4-(2-(2-Aminopyridin-3-yl)-5-(1H-1,2,4-triazol-1-yl)-3H-imidazo[4,5-b]pyridin-3-yl)phenyl)methanol (150 mg) was obtained as a light yellow solid. MS: m/z=385.2 [M+H]⁺.

### Step 4: 3-(3-(4-(Chloromethyl)phenyl)-5-(1H-1,2,4-triazol-1-yl)-3H-imidazo[4,5-b]pyridin-2-yl)pyridin-2-amine

To a solution of (4-(2-(2-aminopyridin-3-yl)-5-(1H-1,2,4-triazol-1-yl)-3H-imidazo[4,5-b]pyridin-3-yl)phenyl)methanol (150 mg, 390 μmol) in CH₂Cl₂ (3 mL) was added SOCl₂ (0.1 mL). The mixture was stirred at 40° C. for 0.5 hr. The mixture was concentrated under reduced pressure to give 3-(3-(4-(chloromethyl)phenyl)-5-(1H-1,2,4-triazol-1-yl)-3H-imidazo[4,5-b]pyridin-2-yl)pyridin-2-amine (Intermediate 253, 150 mg, HCl salt) was obtained as a light yellow solid. MS: m/z=403.2, 405.1 [M+H]⁺.

### Intermediate 254 & 255: 4-((3-Nitro-6-(1H-1,2,3-triazol-1-yl)pyridin-2-yl)amino)benzyl acetate & 4-((3-Nitro-6-(2H-1,2,3-triazol-2-yl)pyridin-2-yl)amino)benzyl acetate

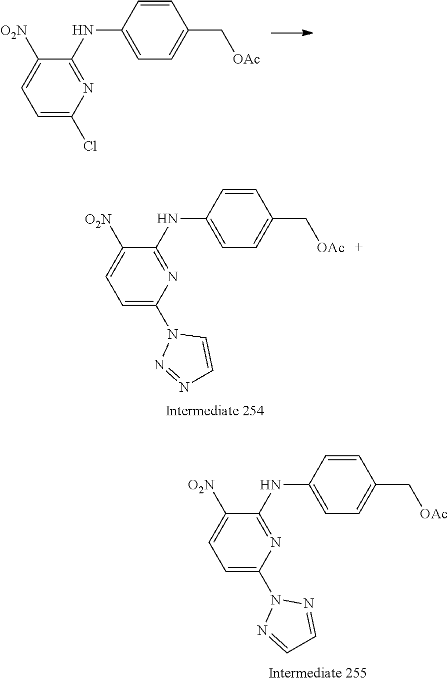

To a solution of 4-((6-chloro-3-nitropyridin-2-yl)amino)benzyl acetate (3 g, 9.33 mmol) 11H-1,2,3-triazole (644 mg, 9.33 mmol) in DMSO (30 mL) was added DIEA (4.87 mL, 28.0 mmol). The mixture was degassed and purged with N₂ for 3 times and stirred at 100° C. for 2 hr under N₂. The reaction mixture was quenched with H₂O (100 mL) at 25° C. and extracted with CH₂Cl₂ (100 mL×2). The combined organic layers were washed with brine (100 mL×3), dried over Na₂SO₄, filtered, and concentrated under reduced pressure. After purified by prep-HPLC (column: Phenomenex luna C18 (250×70 mm, 15 um): mobile phase: [water (NH₄HCO₃)-ACN]; gradient: 35%-65% B over 22 min), 4-((3-nitro-6-(1H-1,2,3-triazol-1-yl)pyridin-2-yl)amino)benzyl acetate (Intermediate 254, 360 mg, yield 10%) was obtained as a yellow solid. MS: m/z=355.0 [M+H]+. ¹H NMR (400 MHz, Chloroform-d) 10.30 (br s, 1H), 8.77 (d, J=8.8 Hz, 1H), 8.38-8.32 (m, 1H), 7.85-7.81 (m, 1H), 7.74 (d, J=8.8 Hz, 1H), 7.58 (d, J=8.0 Hz, 2H), 7.45 (d, J=8.0 Hz, 2H), 5.15 (s, 2H), 2.14 (s, 3H). 4-((3-Nitro-6-(2H-1,2,3-triazol-2-yl)pyridin-2-yl)amino)benzyl acetate (Intermediate 255, 840 mg, yield 24%) was obtained as a red solid. MS: m/z=355.0 [M+H]+. ¹H NMR (400 MHz, Chloroform-d) 10.52 (br s, 1H), 8.73 (dd, J=9.2, 1.6 Hz, 1H), 8.01-7.93 (m, 4H), 7.59 (dd, J=9.2, 2.0 Hz, 1H), 7.50-7.40 (m, 2H), 5.12 (s, 2H), 2.11 (s, 3H).

### Intermediate 256: 3-(3-(4-(Chloromethyl)phenyl)-5-(1H-1,2,3-triazol-1-yl)-3H-imidazo[4,5-b]pyridin-2-yl)pyridin-2-amine

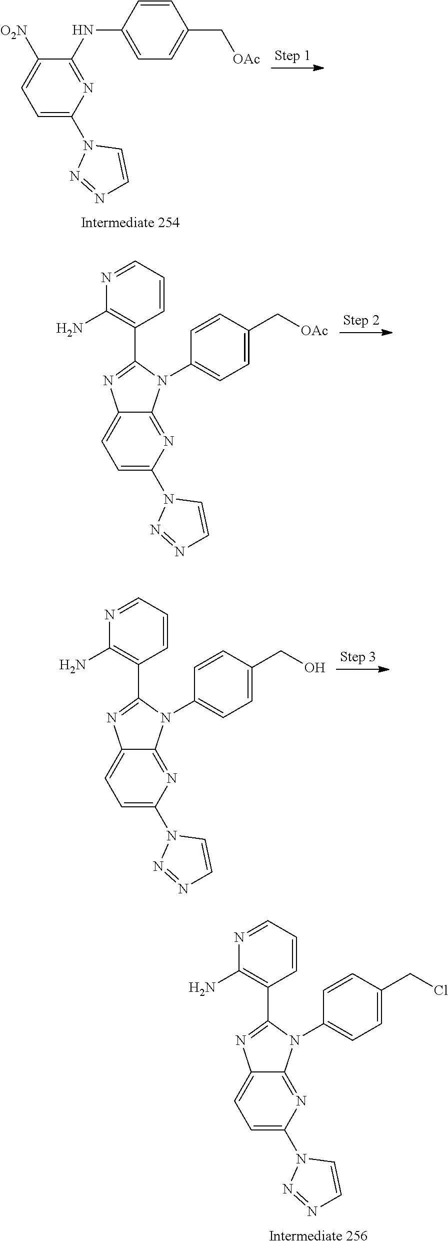 [SMILES: CC(=O)OCc1ccc(Nc2nc(-n3ccnn3)ccc2[N+](=O)[O-])cc1 ; CC(=O)OCc1ccc(-n2c(-c3cccnc3N)nc3ccc(-n4ccnn4)nc32)cc1 ; Nc1ncccc1-c1nc2ccc(-n3ccnn3)nc2n1-c1ccc(CO)cc1 ; Nc1ncccc1-c1nc2ccc(-n3ccnn3)nc2n1-c1ccc(CCl)cc1]

### Step 1:4-(2-(2-Aminopyridin-3-yl)-5-(1H-1,2,3-triazol-1-yl)-3H-imidazo[4,5-b]pyridin-3-yl)benzyl acetate

To a solution of Intermediate 254 (300 mg, 847 μmol) in DMSO (5 mL) were added Na₂S₂O₄ (442 mg, 2.54 mmol) and 2-aminonicotinaldehyde (114 mg, 931 μmol). The mixture was degassed and purged with N₂ for 3 times and stirred at 100° C. for 16 hr under N₂. The reaction mixture was poured into H₂O (50 mL). The mixture was extracted with CH₂Cl₂ (200 mL×2). The combined organic layers were washed with brine (100 mL×3), dried over Na₂SO₄, filtered and concentrated under reduced pressure. After purified by silica gel flash chromatography (Ethyl acetate in petroleum ether=50% to 80%), 4-(2-(2-aminopyridin-3-yl)-5-(1H-1,2,3-triazol-1-yl)-3H-imidazo[4,5-b]pyridin-3-yl)benzyl acetate (51 mg, yield 13%) was obtained as an orange solid. MS: m/z=427.1 [M+H]+. ¹H NMR (400 MHz, Chloroform-d) δ 8.44 (s, 1H), 8.28 (d, J=2.8 Hz, 2H), 8.16-8.05 (m, 1H), 7.78 (d, J=1.6 Hz, 1H), 7.58-7.48 (m, 2H), 7.45-7.36 (m, 2H), 7.09 (d, J=7.6 Hz, 1H), 6.62 (br s, 2H), 6.45-6.35 (m, 1H), 5.24 (s, 2H), 2.18 (s, 3H).

### Step 2: (4-(2-(2-Aminopyridin-3-yl)-5-(1H-1,2,3-triazol-1-yl)-3H-imidazo[4,5-b]pyridin-3-yl)phenyl)methanol

To a solution of 4-(2-(2-aminopyridin-3-yl)-5-(1H-1,2,3-triazol-1-yl)-3H-imidazo[4,5-b]pyridin-3-yl)benzyl acetate (51 mg, 120 μmol) in THF (0.5 mL), H₂O (0.5 mL), and MeOH (0.5 mL) was added K₂CO₃ (33.1 mg, 239 μmol). The mixture was degassed and purged with N₂ for 3 times and stirred at 25° C. for 0.5 hr under N₂. The reaction mixture was filtered and concentrated under reduced pressure. (4-(2-(2-Aminopyridin-3-yl)-5-(1H-1,2,3-triazol-1-yl)-3H-imidazo[4,5-b]pyridin-3-yl)phenyl)methanol (46.0 mg, crude) was obtained as a yellow solid which was used into the next step directly. MS: m/z=385.2 [M+H]⁺.

### Step 3: 3-(3-(4-(Chloromethyl)phenyl)-5-(1H-1,2,3-triazol-1-yl)-3H-imidazo[4,5-b]pyridin-2-yl)pyridin-2-amine

To a solution of (4-(2-(2-aminopyridin-3-yl)-5-(1H-1,2,3-triazol-1-yl)-3H-imidazo[4,5-b]pyridin-3-yl)phenyl)methanol (46.0 mg, 120 μmol) in CH₂Cl₂ (1 mL) was added SOCl₂ (142 mg, 1.20 mmol). The mixture was stirred at 25° C. for 0.5 hr. The reaction mixture was concentrated under reduced pressure to give 3-(3-(4-(chloromethyl)phenyl)-5-(1H-1,2,3-triazol-1-yl)-3H-imidazo[4,5-b]pyridin-2-yl)pyridin-2-amine (Intermediate 256, 48.2 mg, crude, HCl) as a yellow solid, which was used in the next step. MS: m/z=403.1 [M+H]⁺.

### Intermediate 257: 3-(3-(4-(Chloromethyl)phenyl)-5-(2H-1,2,3-triazol-2-yl)-3H-imidazo[4,5-b]pyridin-2-yl)pyridin-2-amine

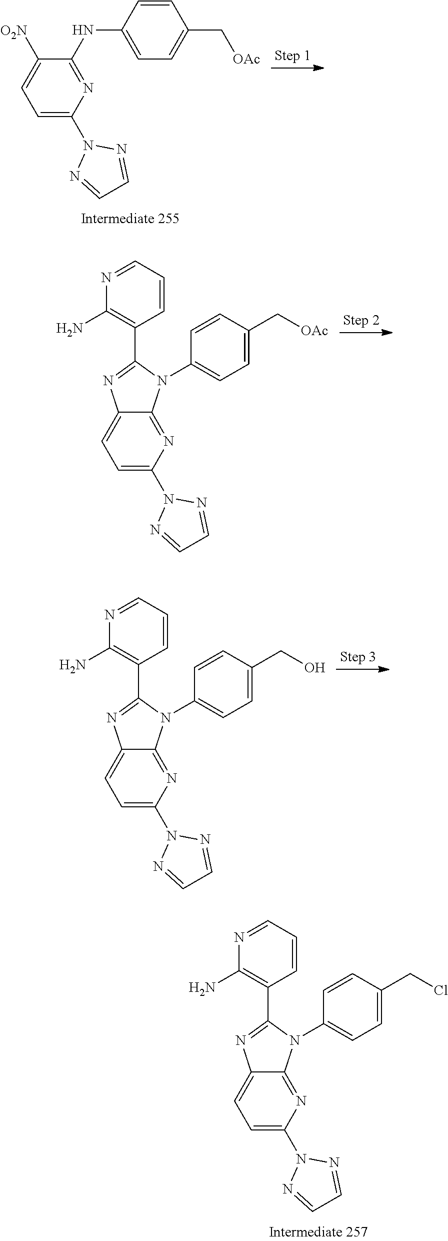 [SMILES: CC(=O)OCc1ccc(Nc2nc(-n3nccn3)ccc2[N+](=O)[O-])cc1 ; CC(=O)OCc1ccc(-n2c(-c3cccnc3N)nc3ccc(-n4nccn4)nc32)cc1 ; Nc1ncccc1-c1nc2ccc(-n3nccn3)nc2n1-c1ccc(CO)cc1 ; Nc1ncccc1-c1nc2ccc(-n3nccn3)nc2n1-c1ccc(CCl)cc1]

### Step 1: 4-(2-(2-Aminopyridin-3-yl)-5-(2H-1,2,3-triazol-2-yl)-3H-imidazo[4,5-b]pyridin-3-yl)benzyl acetate

To a solution of Intermediate 255 (750 mg, 2.12 mmol) in DMSO (10 mL) were added Na₂S₂O₄ (1.11 g, 6.35 mmol) and 2-aminonicotinaldehyde (284 mg, 2.33 mmol). The mixture was degassed and purged with N₂ for 3 times and stirred at 100° C. for 16 hr under N₂. The reaction mixture was poured into H₂O (50 mL). The mixture was extracted with CH₂Cl₂ (200 mL×2). The combined organic layers were washed with brine (100 mL×3), dried over Na₂SO₄, filtered and concentrated under reduced pressure. After purified by silica gel flash chromatography (Ethyl acetate in petroleum ether=50% to 80%), 4-(2-(2-aminopyridin-3-yl)-5-(2H-1,2,3-triazol-2-yl)-3H-imidazo[4,5-b]pyridin-3-yl)benzyl acetate (102 mg, yield 9.7%) was obtained as an orange solid. MS: m/z=449.1 [M+Na]⁺. ¹H NMR (400 MHz, Chloroform-d) δ 8.30-8.21 (m, 1H), 8.17-8.05 (m, 2H), 7.88 (d, J=1.6 Hz, 2H), 7.58-7.39 (m, 4H), 7.11-7.02 (m, 1H), 6.57 (br s, 2H), 6.46-6.35 (m, 1H), 5.19 (s, 2H), 2.16 (s, 3H).

### Step 2: (4-(2-(2-Aminopyridin-3-yl)-5-(2H-1,2,3-triazol-2-yl)-3H-imidazo[4,5-b]pyridin-3-yl)phenyl)methanol

To a solution of 4-(2-(2-aminopyridin-3-yl)-5-(2H-1,2,3-triazol-2-yl)-3H-imidazo[4,5-b]pyridin-3-yl)benzyl acetate (100 mg, 235 μmol) in THF (1 mL), H₂O (1 mL), and MeOH (1 mL) was added K₂CO₃ (64.8 mg, 469 μmol). The mixture was degassed and purged with N₂ for 3 times and stirred at 25° C. for 0.5 hr under N₂. The reaction mixture was filtered and concentrated under reduced pressure to give (4-(2-(2-aminopyridin-3-yl)-5-(2H-1,2,3-triazol-2-yl)-3H-imidazo[4,5-b]pyridin-3-yl)phenyl)methanol (90.1 mg, crude) as a yellow solid, which was used in the next step directly. MS: m/z=385.1 [M+H]⁺.

### Step 3: 3-(3-(4-(Chloromethyl)phenyl)-5-(2H-1,2,3-triazol-2-yl)-3H-imidazo[4,5-b]pyridin-2-yl)pyridin-2-amine

To a solution of (4-(2-(2-aminopyridin-3-yl)-5-(2H-1,2,3-triazol-2-yl)-3H-imidazo[4,5-b]pyridin-3-yl)phenyl)methanol (90.1 mg, 235 μmol) in CH₂Cl₂ (1 mL) was added SOCl₂ (279 mg, 2.34 mmol). The mixture was degassed and purged with N₂ for 3 times and stirred at 25° C. for 0.5 hr under N₂. The reaction mixture was concentrated under reduced pressure to give 3-(3-(4-(chloromethyl)phenyl)-5-(2H-1,2,3-triazol-2-yl)-3H-imidazo[4,5-b]pyridin-2-yl)pyridin-2-amine (Intermediate 257, 103 mg, crude, HCl) as a yellow solid which was used into the next step directly. MS: m/z=403.1 [M+H]⁺.

### Intermediate 258 & 259: 4-((6-(4-Methyl-1H-1,2,3-triazol-1-yl)-3-nitropyridin-2-yl)amino)benzyl acetate & 4-((6-(4-Methyl-2H-1,2,3-triazol-2-yl)-3-nitropyridin-2-yl)amino)benzyl acetate

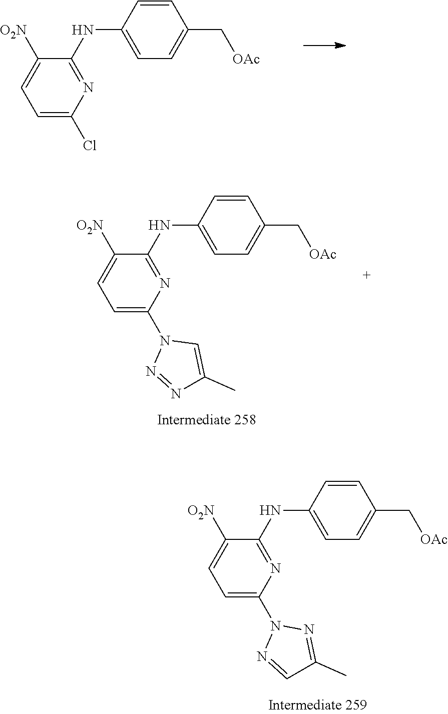 [SMILES: CC(=O)OCc1ccc(Nc2nc(Cl)ccc2[N+](=O)[O-])cc1 ; CC(=O)OCc1ccc(Nc2nc(-n3cc(C)nn3)ccc2[N+](=O)[O-])cc1 ; CC(=O)OCc1ccc(Nc2nc(-n3ncc(C)n3)ccc2[N+](=O)[O-])cc1]

To a solution of 4-((6-chloro-3-nitropyridin-2-yl)amino)benzyl acetate (refer to Intermediate 178 for detail procedures, 3 g, 9.33 mmol) and 4-methyl-1H-1,2,3-triazole (775 mg, 9.33 mmol) in DMSO (20 mL) was added DIEA (3.62 g, 28 mmol). The mixture was stirred at 80° C. for 16 hr. The reaction mixture was quenched with H₂O (100 mL) at 25° C. and extracted with CH₂Cl₂ (100 mL×2). The combined organic layers were washed with brine (100 mL×3), dried over Na₂SO₄, filtered and concentrated under reduced pressure. After purified by prep-HPLC (column: Phenomenex Cis 150×25 mm×10 μm; mobile phase: [water (NH₄HCO₃)-ACN]; gradient: 35%-65% B over 22 min), 4-((6-(4-methyl-1H-1,2,3-triazol-1-yl)-3-nitropyridin-2-yl)amino)benzyl acetate (Intermediate 258, 720 mg, yield: 20%) was obtained as a red solid. MS: m/z=391.2 [M+Na]⁺. ¹H NMR (400 MHz, Dimethylsulfoxide-d₆) δ 10.27 (s, 1H), 8.78 (d, J=8.8 Hz, 1H), 8.23-8.21 (m, 1H), 7.70 (d, J=8.4 Hz, 2H), 7.58 (d, J=8.8 Hz, 1H), 7.45 (d, J=8.4 Hz, 2H), 5.10 (s, 2H), 2.36 (s, 3H), 2.09 (s, 3H). 4-((6-(4-Methyl-2H-1,2,3-triazol-2-yl)-3-nitropyridin-2-yl)amino)benzyl acetate (Intermediate 259, 1.4 g, yield: 39%) was obtained as a red solid. MS: m/z=369.1 [M+Na]. ¹H NMR (400 MHz, Dimethylsulfoxide-d₆) δ 10.30 (s, 1H), 8.71 (d, J=9.2 Hz, 1H), 8.1 (s, 1H), 7.96 (d, J=8.4 Hz, 2H), 7.46 (d, J=8.8 Hz, 1H), 7.38 (d, J=8.4 Hz, 2H), 5.07 (s, 2H), 2.36 (s, 3H), 2.07 (s, 3H).

### Intermediate 260: 3-(3-(4-(Chloromethyl)phenyl)-5-(4-methyl-1H-1,2,3-triazol-1-yl)-3H-imidazo[4,5-b]pyridin-2-yl)pyridin-2-amine

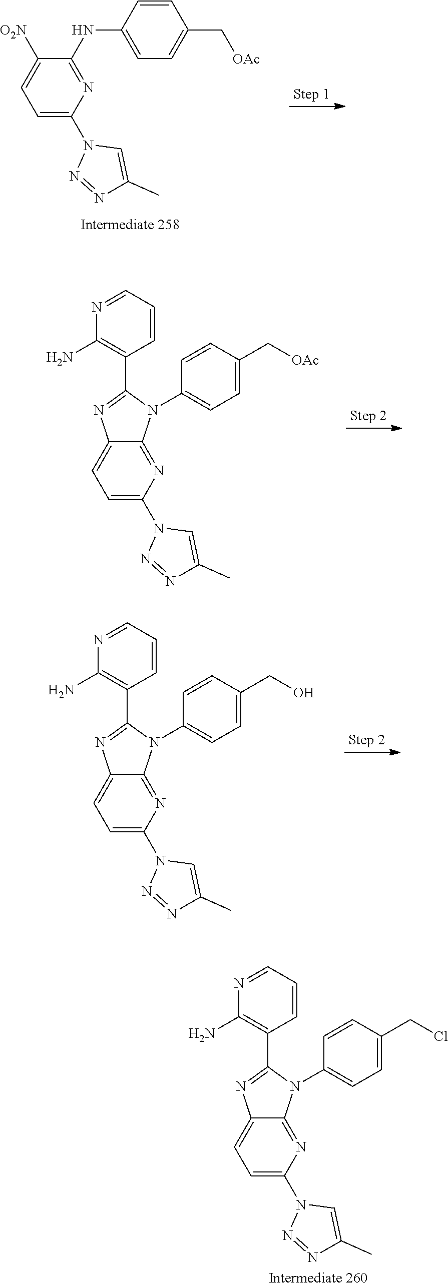 [SMILES: CC(=O)OCc1ccc(Nc2nc(-n3cc(C)nn3)ccc2[N+](=O)[O-])cc1 ; CC(=O)OCc1ccc(-n2c(-c3cccnc3N)nc3ccc(-n4cc(C)nn4)nc32)cc1 ; Cc1cn(-c2ccc3nc(-c4cccnc4N)n(-c4ccc(CO)cc4)c3n2)nn1 ; Cc1cn(-c2ccc3nc(-c4cccnc4N)n(-c4ccc(CCl)cc4)c3n2)nn1]

### Step 1: 4-(2-(2-Aminopyridin-3-yl)-5-(4-methyl-1H-1,2,3-triazol-1-yl)-3H-imidazo[4,5-b]pyridin-3-yl)benzyl acetate

To a solution of Intermediate 258 (650 mg, 1.7 mmol) in DMSO (10 mL) were added 2-aminonicotinaldehyde (258 mg, 2.0 mmol) and Na₂S₂O₄ (1.2 g, 7.0 mmol) at 25° C. The mixture was stirred at 100° C. for 16 hr. The reaction mixture was poured into H₂O (50 mL) and extracted with CH₂Cl₂ (200 mL×2). The combined organic layers were washed with brine (400 mL), dried over anhydrous Na₂SO₄, filtered and concentrated. After purified by silica gel flash chromatography (Eluent of 1%˜3% MeOH in CH₂Cl₂), 4-(2-(2-aminopyridin-3-yl)-5-(4-methyl-1H-1,2,3-triazol-1-yl)-3H-imidazo[4,5-b]pyridin-3-yl)benzyl acetate (720 mg, yield: 61%) was obtained as a yellow solid. MS: m/z=441.0 [M+H]+. ¹H NMR (400 MHz, Dimethylsulfoxide-d₆) δ 8.47 (d, J=8.4 Hz, 1H), 8.35 (s, 1H), 8.08 (d, J=8.4 Hz, 1H), 8.01 (d, =4.0 Hz, 1H), 7.55-7.50 (m, 4H), 7.26 (d, J=7.6 Hz, 1H), 6.81 (br s, 2H), 6.48-6.42 (m, 1H), 5.17 (s, 2H), 2.32 (s, 3H), 2.12 (s, 3H)

### Step 2: (4-(2-(2-Aminopyridin-3-yl)-5-(4-methyl-1H-1,2,3-triazol-1-yl)-3H-imidazo[4,5-b]pyridin-3-yl)phenyl)methanol

To a solution of 4-(2-(2-aminopyridin-3-yl)-5-(4-methyl-1H-1,2,3-triazol-1-yl)-3H-imidazo[4,5-b]pyridin-3-yl)benzyl acetate (200 mg, 454 μmol) in MeOH (5 mL), H₂O (1 mL) and THF (5 mL) was added K₂CO₃ (125 mg, 908 μmol). The mixture was stirred at 25° C. for 1 hr. The reaction mixture was quenched with H₂O (20 mL) at 25° C. and extracted with CH₂Cl₂ (15 mL×2). The combined organic layers were washed with brine (30 mL×2), dried over Na₂SO₄, filtered and concentrated under reduced pressure. (4-(2-(2-Aminopyridin-3-yl)-5-(4-methyl-11H-1,2,3-triazol-1-yl)-3H-imidazo[4,5-b]pyridin-3-yl)phenyl)methanol (180 mg, crude) was obtained as a yellow solid. MS: m/z=399.0 [M+H]⁺.

### Step 3: 3-(3-(4-(Chloromethyl)phenyl)-5-(4-methyl-1H-1,2,3-triazol-1-yl)-3H-imidazo[4,5-b]pyridin-2-yl)pyridin-2-amine

To a solution of (4-(2-(2-aminopyridin-3-yl)-5-(4-methyl-1H-1,2,3-triazol-1-yl)-3H-imidazo[4,5-b]pyridin-3-yl)phenyl)methanol (132 mg, 331 μmol) in CH₂Cl₂ (2 mL) was added SOCl₂ (1 mL). The mixture was stirred at 25° C. for 0.5 hr. The reaction mixture was concentrated under reduced pressure to give 3-(3-(4-(chloromethyl)phenyl)-5-(4-methyl-1H-1,2,3-triazol-1-yl)-3H-imidazo[4,5-b]pyridin-2-yl)pyridin-2-amine (Intermediate 260, 146 mg, HCl salt) was obtained as a gray solid. MS: m/z=417.1, 419.1 [M+H]⁺.

### Intermediate 261: 3-(3-(4-(Chloromethyl)phenyl)-5-(oxazol-4-yl)-3H-imidazo[4,5-b]pyridin-2-yl)pyridin-2-amine

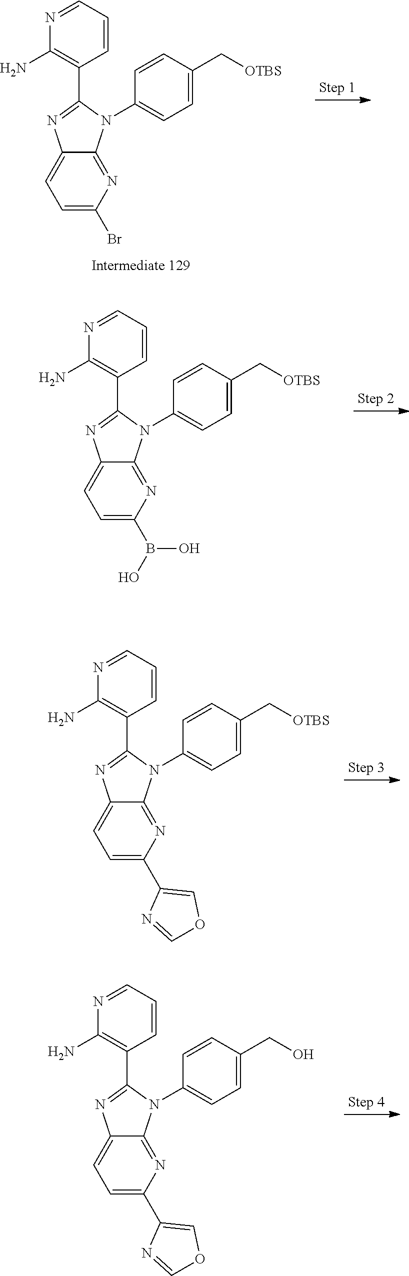 [SMILES: CC(C)(C)[Si](C)(C)OCc1ccc(-n2c(-c3cccnc3N)nc3ccc(Br)nc32)cc1 ; CC(C)(C)[Si](C)(C)OCc1ccc(-n2c(-c3cccnc3N)nc3ccc(B(O)O)nc32)cc1 ; CC(C)(C)[Si](C)(C)OCc1ccc(-n2c(-c3cccnc3N)nc3ccc(-c4cocn4)nc32)cc1 ; Nc1ncccc1-c1nc2ccc(-c3cocn3)nc2n1-c1ccc(CO)cc1]

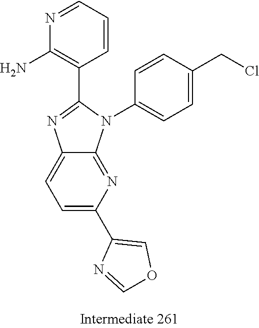 [SMILES: Nc1ncccc1-c1nc2ccc(-c3cocn3)nc2n1-c1ccc(CCl)cc1]

### Step 1: (2-(2-Aminopyridin-3-yl)-3-(4-(((tert-butyldimethylsilyl)oxy)methyl)phenyl)-3H-imidazo[4,5-b]pyridin-5-yl)boronic acid

A mixture of Intermediate 129 (500 mg, 979 μmol), 4,4,4′,4′,5,5,5′,5′-octamethyl-2,2′-bi(1,3,2-dioxaborolane) (498 mg, 1.96 mmol), KOAc (288 mg, 2.94 mmol) and Pd(dppf)Cl₂ (71.7 mg, 97.9 μmol) in 1,4-dioxane (5 mL) was degassed and purged with N₂ three times. The mixture was stirred at 90° C. for 8 hr under N₂ atmosphere. (2-(2-Aminopyridin-3-yl)-3-(4-(((tert-butyldimethylsilyl)oxy)methyl)phenyl)-3H-imidazo[4,5-b]pyridin-5-yl)boronic acid was obtained as a black liquid, which was used for next step directly and without work-up and purification. MS: m/z=476.2 [M+H]⁺.

### Step 2: 3-(3-(4-(((tert-Butyldimethylsilyl)oxy)methyl)phenyl)-5-(oxazol-4-yl)-3H-imidazo[4,5-b]pyridin-2-yl)pyridin-2-amine

A mixture of (2-(2-aminopyridin-3-yl)-3-(4-(((tert-butyldimethylsilyl)oxy)methyl)phenyl)-3H-imidazo[4,5-b]pyridin-5-yl)boronic acid (466 mg, 980 μmol), 4-bromooxazole (145 mg, 980 μmol), Pd(dppf)Cl₂ (71.7 mg, 98 μmol) and Cs₂CO₃ (958 mg, 2.94 mmol) in 1,4-dioxane (5 mL) and H₂O (1 mL) was degassed and purged with N₂ three times. The mixture was stirred at 90° C. for 16 hr under N₂ atmosphere. The reaction mixture was quenched with H₂O (5 mL) and extracted with CH₂Cl₂ (15 mL×2). The combined organic layers were washed with brine (30 mL), dried over Na₂SO₄, filtered and concentrated under reduced pressure. After purified by silica gel flash chromatography (Eluent of 30%˜60/EtOAc in petroleum ether), 3-(3-(4-(((tert-butyldimethylsilyl)oxy)methyl)phenyl)-5-(oxazol-4-yl)-3H-imidazo[4,5-b]pyridin-2-yl)pyridin-2-amine (150 mg, yield: 31% for two steps) was obtained as a black solid. MS: m/z=499.3 [M+H]⁺. ¹H NMR (400 MHz, Chloroform-d) δ 8.17-7.90 (m, 4H), 7.54-7.44 (m, 2H), 7.39-7.33 (m, 2H), 7.16-7.06 (m, 1H), 6.67-6.65 (m, 2H), 6.36 (dd, J=7.6, 4.8 Hz, 1H), 4.92-4.81 (m, 2H), 1.02-0.95 (m, 9H), 0.24-0.04 (m, 6H).

### Step 3: (4-(2-(2-Aminopyridin-3-yl)-5-(oxazol-4-yl)-3H-imidazo[4,5-b]pyridin-3-yl)phenyl)methanol

To a solution of 3-(3-(4-(((tert-butyldimethylsilyl)oxy)methyl)phenyl)-5-(oxazol-4-yl)-3H-imidazo[4,5-b]pyridin-2-yl)pyridin-2-amine (150 mg, 301 μmol) in THF (5 mL) was added TBAF (236 mg, 1 M in THF). The mixture was stirred at 25° C. for 1 hr. The reaction mixture was quenched with H₂O (5 mL) at 25° C. and extracted with EtOAc (15 mL×3). The combined organic layers were washed with brine (50 mL×3), dried over Na₂SO₄, filtered and concentrated under reduced pressure. (4-(2-(2-Aminopyridin-3-yl)-5-(oxazol-4-yl)-3H-imidazo[4,5-b]pyridin-3-yl)phenyl)methanol (130 mg) was obtained as a brown solid. MS: m/z=385.1 [M+H]+.

### Step 4: 3-(3-(4-(Chloromethyl)phenyl)-5-(oxazol-4-yl)-3H-imidazo[4,5-b]pyridin-2-yl)pyridin-2-amine

To a solution of (4-(2-(2-aminopyridin-3-yl)-5-(oxazol-4-yl)-3H-imidazo[4,5-b]pyridin-3-yl)phenyl)methanol (120 mg, 312 μmol) in CH₂Cl₂ (5 mL) was added SOCl₂ (112 mg, 937 μmol). The mixture was stirred at 40° C. for 0.5 hr. The reaction was concentrated under reduced pressure to give 3-(3-(4-(chloromethyl)phenyl)-5-(oxazol-4-yl)-3H-imidazo[4,5-b]pyridin-2-yl)pyridin-2-amine (Intermediate 261, 122 mg, HCl salt) as a yellow solid. MS: m/z=403.0 [M+H]⁺.

### Intermediate 262: 3-(3-(4-(Chloromethyl)phenyl)-5-(4-fluoropyridin-3-yl)-3H-imidazo[4,5-b]pyridin-2-yl)pyridin-2-amine

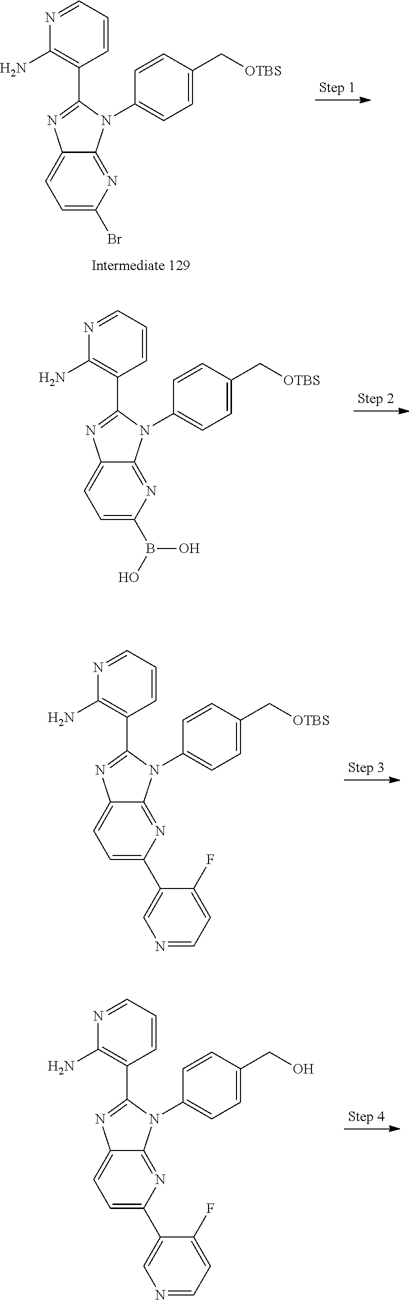 [SMILES: CC(C)(C)[Si](C)(C)OCc1ccc(-n2c(-c3cccnc3N)nc3ccc(Br)nc32)cc1 ; CC(C)(C)[Si](C)(C)OCc1ccc(-n2c(-c3cccnc3N)nc3ccc(B(O)O)nc32)cc1 ; CC(C)(C)[Si](C)(C)OCc1ccc(-n2c(-c3cccnc3N)nc3ccc(-c4cnccc4F)nc32)cc1 ; Nc1ncccc1-c1nc2ccc(-c3cnccc3F)nc2n1-c1ccc(CO)cc1]

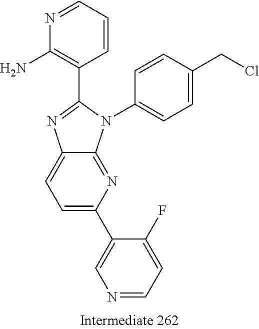 [SMILES: Nc1ncccc1-c1nc2ccc(-c3cnccc3F)nc2n1-c1ccc(CCl)cc1]

### Step 1: (2-(2-Aminopyridin-3-yl)-3-(4-(((tert-butyldimethylsilyl)oxy)methyl)phenyl)-3H-imidazo[4,5-b]pyridin-5-yl)boronic acid

To a solution of Intermediate 129 (500 mg, 979 μmol) in 1,4-dioxane (6 mL) were added 4,4,4′,4′,5,5,5′,5′-octamethyl-2,2′-bi(1,3,2-dioxaborolane) (497 mg, 2.0 mmol), KOAc (288 mg, 2.9 mmol) and Pd(dppf)Cl₂ (71.7 mg, 97.9 μmol). The reaction mxiture was stirred at 85° C. for 2 hr under N₂. (2-(2-Aminopyridin-3-yl)-3-(4-(((tert-butyldimethylsilyl)oxy)methyl)phenyl)-3H-imidazo[4,5-b]pyridin-5-yl)boronic acid (466 mg, 979 μmol, crude) was obtained as brown liquid and used to the next step without further purification. MS: m/z=476.2 [M+H]⁺.

### Step 2: 3-(3-(4-(((Tert-butyldimethylsilyl)oxy)methyl)phenyl)-5-(4-fluoropyridin-3-yl)-3H-imidazo[4,5-b]pyridin-2-yl)pyridin-2-amine

To a solution of (2-(2-aminopyridin-3-yl)-3-(4-(((tert-butyldimethylsilyl)oxy)methyl)phenyl)-3H-imidazo[4,5-b]pyridin-5-yl)boronic acid (512 mg, 1.1 mmol) in 1,4-dioxane (5 mL) and H₂O (1 mL) were added 3-bromo-4-fluoropyridine (227 mg, 1.3 mmol), Cs₂CO₃ (1.1 g, 3.2 mmol), and Pd(dppf)Cl₂ (78.8 mg, 108 μmol). The reaction mixture was stirred at 100° C. for 2 hr under N₂. The mixture was diluted with H₂O (10 mL) and extracted with EtOAc (6 mL×2). The combined organic layers were washed with brine (10 mL), dried over anhydrous Na₂SO₄, filtered and concentrated. After purified by silica gel flash chromatography (Eluent of 0˜51% EtOAc in petroleum ether), 3-(3-(4-(((tert-butyldimethylsilyl)oxy)methyl)phenyl)-5-(4-fluoropyridin-3-yl)-3H-imidazo[4,5-b]pyridin-2-yl)pyridin-2-amine (567 mg, yield: 97.7%) was obtained as a black solid. MS: m/z=527.2 [M+H]⁺. ¹H NMR (400 MHz, Chloroform-d) δ 9.20 (d, J=10.4 Hz, 1H), 8.54 (dd, J=7.2, 5.6 Hz, 1H), 8.16 (d, J=8.4 Hz, 1H), 8.07 (dd, J=4.8, 1.6 Hz, 1H), 7.84 (dd, J=8.4, 1.6 Hz, 1H), 7.49 (d, J=8.4 Hz, 2H), 7.38 (d, J=8.4 Hz, 2H), 7.15 (dd, J=8.0, 2.0 Hz, 1H), 7.11 (dd, J=10.8, 5.2 Hz, 1H), 6.68 (br s, 2H), 6.38 (dd, J=7.6, 4.8 Hz, 1H), 4.85 (s, 2H), 0.97 (s, 9H), 0.14 (s, 6H). ¹⁹F NMR (400 MHz, Dimethylsulfoxide-d₆) δ −107.329.

### Step 3: (4-(2-(2-Aminopyridin-3-yl)-5-(4-fluoropyridin-3-yl)-3H-imidazo[4,5-b]pyridin-3-yl)phenyl)methanol

To a solution of 3-(3-(4-(((tert-butyldimethylsilyl)oxy)methyl)phenyl)-5-(4-fluoropyridin-3-yl)-3H-imidazo[4,5-b]pyridin-2-yl)pyridin-2-amine (350 mg, 665 μmol) in THF (4 mL) was added TBAF (1 M in THE, 1.06 mL). The mixture was stirred at 25° C. for 1 hr under N₂. The mixture was diluted with H₂O (10 mL) was added to the mixture, the aqueous layer was extracted with EtOAc (5 mL×3). The combined organic phase was washed with brine (20 mL), dried over anhydrous Na₂SO₄, filtered and concentrated. (4-(2-(2-Aminopyridin-3-yl)-5-(4-fluoropyridin-3-yl)-3H-imidazo[4,5-b]pyridin-3-yl)phenyl)methanol (274 mg, 665 μmol, crude) was obtained as a brown solid, which was used to the next step without further purification. MS: m/z=413.1 [M+H]⁺.

### Step 4: 3-(3-(4-(Chloromethyl)phenyl)-5-(4-fluoropyridin-3-yl)-3H-imidazo[4,5-b]pyridin-2-yl)pyridin-2-amine

To a solution of (4-(2-(2-aminopyridin-3-yl)-5-(4-fluoropyridin-3-yl)-3H-imidazo[4,5-b]pyridin-3-yl)phenyl)methanol (274 mg, 664 μmol) in CH₂Cl₂ (3 mL) was added SOCl₂ (237 mg, 2.0 mmol). The reaction mixture was stirred at 25° C. for 12 hr under N₂. The mixture was concentrated to give 3-(3-(4-(chloromethyl)phenyl)-5-(4-fluoropyridin-3-yl)-3H-imidazo[4,5-b]pyridin-2-yl)pyridin-2-amine (Intermediate 262, 286 mg, 664 μmol, crude) as a brown solid, which was used in the next step without further purification. MS: m/z=431.1 [M+H]⁺.

### Intermediate 263: 3-(3-(4-(Chloromethyl)phenyl)-5-(4-methylpyridin-2-yl)-3H-imidazo[4,5-b]pyridin-2-yl)pyridin-2-amine

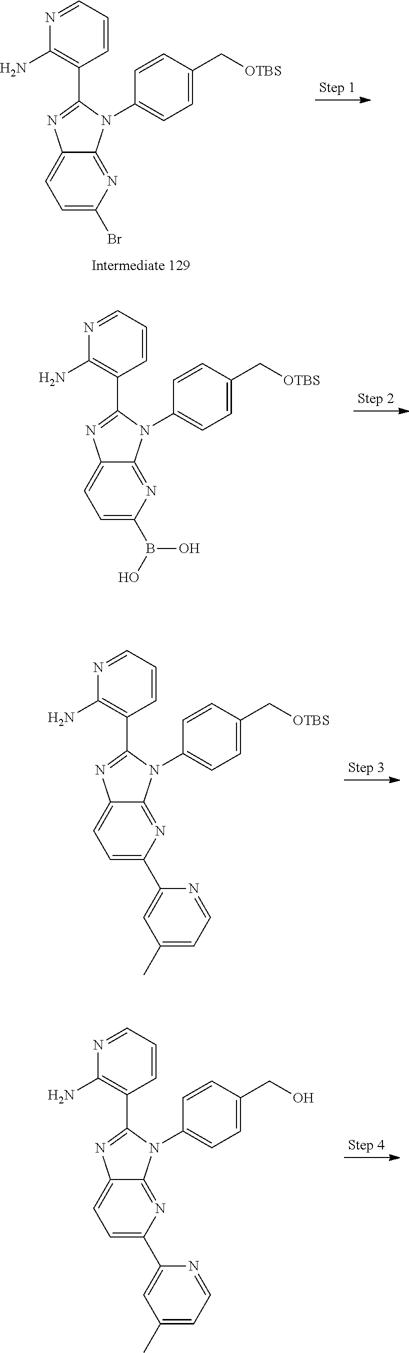 [SMILES: CC(C)(C)[Si](C)(C)OCc1ccc(-n2c(-c3cccnc3N)nc3ccc(Br)nc32)cc1 ; CC(C)(C)[Si](C)(C)OCc1ccc(-n2c(-c3cccnc3N)nc3ccc(B(O)O)nc32)cc1 ; Cc1ccnc(-c2ccc3nc(-c4cccnc4N)n(-c4ccc(CO[Si](C)(C)C(C)(C)C)cc4)c3n2)c1 ; Cc1ccnc(-c2ccc3nc(-c4cccnc4N)n(-c4ccc(CO)cc4)c3n2)c1]

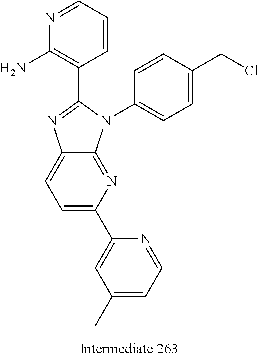 [SMILES: Cc1ccnc(-c2ccc3nc(-c4cccnc4N)n(-c4ccc(CCl)cc4)c3n2)c1]

### Step 1: (2-(2-Aminopyridin-3-yl)-3-(4-(((tert-butyldimethylsilyl)oxy)methyl)phenyl)-3H-imidazo[4,5-b]pyridin-5-yl)boronic acid

A mixture of Intermediate 129 (500 mg, 980 μmol), 4,4,4′,4′,5,5,5′,5′-octamethyl-2,2′-bi(1,3,2-dioxaborolane) (497 mg, 1.96 mmol), KOAc (289 mg, 2.94 mmol) and Pd(dppf)Cl₂ (72 mg, 98.0 μmol) in 1,4-dioxane (5 mL) was degassed and purged with N₂ three times. The mixture was stirred at 80° C. for 4 hr under N₂ atmosphere. (2-(2-Aminopyridin-3-yl)-3-(4-(((tert-butyldimethylsilyl)oxy)methyl)phenyl)-3H-imidazo[4,5-b]pyridin-5-yl)boronic acid was obtained as a black liquid, which was used in the next step without work-up and purification. MS: m/z=476.1 [M+H]⁺.

### Step 2: 3-(3-(4-(((Tert-butyldimethylsilyl)oxy)methyl)phenyl)-5-(4-methylpyridin-2-yl)-3H-imidazo[4,5-b]pyridin-2-yl)pyridin-2-amine

To a solution of (2-(2-aminopyridin-3-yl)-3-(4-(((tert-butyldimethylsilyl)oxy)methyl)phenyl)-3H-imidazo[4,5-b]pyridin-5-yl)boronic acid (490 mg, 1.03 mmol) and 2-bromo-4-methylpyridine (177 mg, 1.03 mmol) in 1,4-dioxane (10 mL) and H₂O (2 mL) were added Pd(dppf)Cl₂ (75.4 mg, 103 μmol) and Cs₂CO₃ (1.01 g, 3.09 mmol) at 25° C. The mixture was degassed and purged with N₂ for 3 times and stirred at 100° C. for 2 hr. The mixture was quenched with H₂O (20 mL) and the aqueous layer was extracted with EtOAc (20 mL×3). The combined organic layers were washed with brine (20 mL), dried over anhydrous Na₂SO₄, filtered and concentrated. After purified by silica gel flash chromatography (EtOAc in petroleum ether=0-40%), 3-(3-(4-(((tert-butyldimethylsilyl)oxy)methyl)phenyl)-5-(4-methylpyridin-2-yl)-3H-imidazo[4,5-b]pyridin-2-yl)pyridin-2-amine (350 mg, 670 μmol, yield: 65% for two steps) was obtained as a green solid. MS: m/z=523.3 [M+H]⁺. ¹H NMR (400 MHz, Dimethylsulfoxide-d₆) δ 8.52 (d, J=4.8 Hz, 1H), 8.46 (d, J=8.4 Hz, 1H), 8.29 (d, J=8.4 Hz, 1H), 7.02-7.99 (m, 1H), 7.99-7.97 (m, 1H), 7.55-7.46 (m, 4H), 7.25-7.17 (m, 2H), 7.04-6.98 (m, 2H), 6.39 (dd, J=7.6, 4.8 Hz, 1H), 4.83 (s, 2H), 2.34 (s, 3H), 0.92 (s, 9H), 0.11 (s, 6H).

### Step 3: (4-(2-(2-Aminopyridin-3-yl)-5-(4-methylpyridin-2-yl)-3H-imidazo[4,5-b]pyridin-3-yl)phenyl)methanol

To a solution of 3-(3-(4-(((tert-butyldimethylsilyl)oxy)methyl)phenyl)-5-(4-methylpyridin-2-yl)-3H-imidazo[4,5-b]pyridin-2-yl)pyridin-2-amine (320 mg, 612 μmol) in THF (5 mL) was added TBAF (1 M in THF, 1.84 mL) at 25° C. The mixture was stirred at 25° C. for 1 hr. The mixture was quenched with H₂O (20 mL) and extracted with EtOAc (20 mL×3). The combined organic layers were washed with brine (20 mL), dried over anhydrous Na₂SO₄, filtered and concentrated. (4-(2-(2-Aminopyridin-3-yl)-5-(4-methylpyridin-2-yl)-3H-imidazo[4,5-b]pyridin-3-yl)phenyl)methanol (250 mg, 612 μmol) was obtained as a brown solid which was used into the next step directly. MS: m/z=409.2 [M+H]⁺.

### Step 4: 3-(3-(4-(Chloromethyl)phenyl)-5-(4-methylpyridin-2-yl)-3H-imidazo[4,5-b]pyridin-2-yl)pyridin-2-amine

To a mixture of (4-(2-(2-aminopyridin-3-yl)-5-(4-methylpyridin-2-yl)-3H-imidazo[4,5-b]pyridin-3-yl)phenyl)methanol (250 mg, 612 μmol) in CH₂Cl₂ (3 mL) was added SOCl₂ (218 mg, 1.84 mmol). The reaction mixture was stirred at 20° C. for 1 hr. The mixture was concentrated under reduced pressure to give 3-(3-(4-(chloromethyl)phenyl)-5-(4-methylpyridin-2-yl)-3H-imidazo[4,5-b]pyridin-2-yl)pyridin-2-amine (Intermediate 263, 261 mg, 611 μmol) as a brown solid. MS: m/z=427.2, 429.2 [M+H]⁺.

### Intermediate 264: 5-bromo-2-(methoxy-d₃)pyridine-6-d

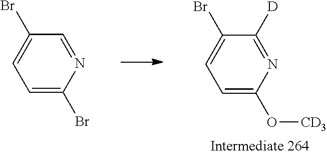 [SMILES: Brc1ccc(Br)nc1 ; [2H]c1nc(OC([2H])([2H])[2H])ccc1Br]

To a solution of 2,5-dibromopyridine (1 g, 4.22 mmol) in CD₃OD (10 mL) was added NaOH (338 mg, 8.44 mmol). The mixture was stirred at 70° C. for 2 hr. The reaction mixture was quenched with H₂O (20 mL) at 25° C. and extracted with CH₂Cl₂ (25 mL×2). The combined organic layers were washed with brine (30 mL), dried over anhydrous Na₂SO₄, filtered and concentrated under reduced pressure. After purified by silica gel flash chromatography (Eluent of 0%˜1% EtOAc in petroleum ether), 5-bromo-2-(methoxy-d₃)pyridine-6-d (Intermediate 264, 600 mg, yield: 74%) was obtained as a colorless oil. MS: m/z=190.9, 192.9 [M+H]⁺. D %: 3D %=66.6%, 4D %=33.4%. ¹H NMR (400 MHz, Dimethylsulfoxide-d₆) δ 8.28 (s, 1H), 7.89 (dd, J=9.2, 2.8 Hz, 1H), 6.86-6.79 (m, 1H).

### Intermediate 265: 3-(3-(4-(Chloromethyl)phenyl)-5-(6-(methoxy-d₃)pyridin-3-yl-2-d)-3H-imidazo[4,5-b]pyridin-2-yl)pyridin-2-amine

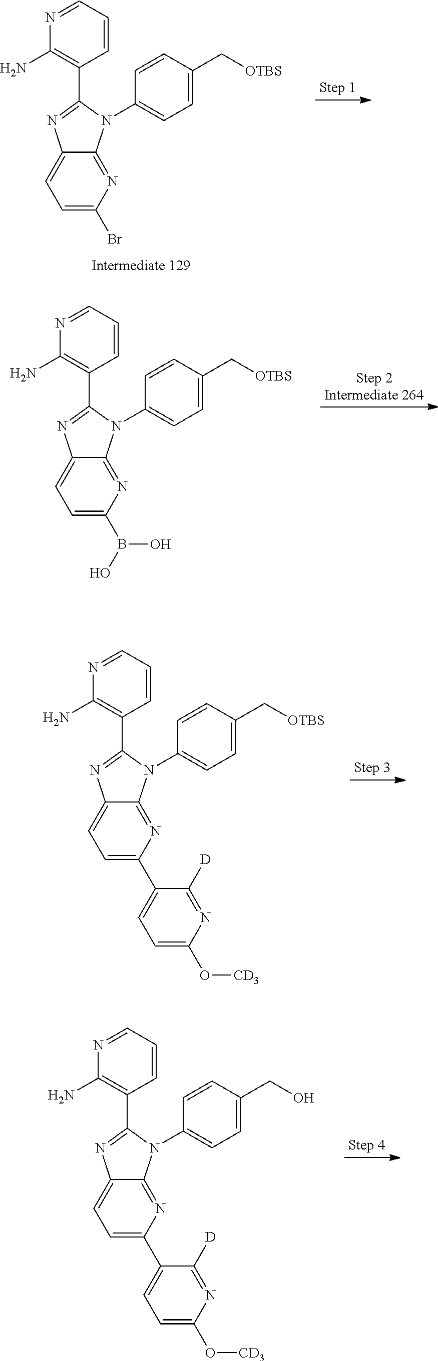 [SMILES: CC(C)(C)[Si](C)(C)OCc1ccc(-n2c(-c3cccnc3N)nc3ccc(Br)nc32)cc1 ; CC(C)(C)[Si](C)(C)OCc1ccc(-n2c(-c3cccnc3N)nc3ccc(B(O)O)nc32)cc1 ; [2H]c1nc(OC([2H])([2H])[2H])ccc1-c1ccc2nc(-c3cccnc3N)n(-c3ccc(CO[Si](C)(C)C(C)(C)C)cc3)c2n1 ; [2H]c1nc(OC([2H])([2H])[2H])ccc1-c1ccc2nc(-c3cccnc3N)n(-c3ccc(CO)cc3)c2n1]

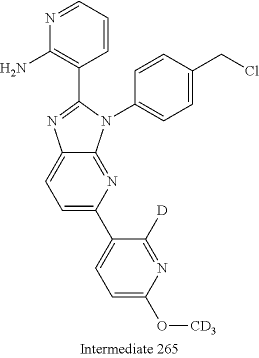 [SMILES: [2H]c1nc(OC([2H])([2H])[2H])ccc1-c1ccc2nc(-c3cccnc3N)n(-c3ccc(CCl)cc3)c2n1]

### Step 1: (2-(2-Aminopyridin-3-yl)-3-(4-(((tert-butyldimethylsilyl)oxy)methyl)phenyl)-3H-imidazo[4,5-b]pyridin-5-yl)boronic acid

A mixture of Intermediate 129 (600 mg, 1.18 mmol), 4,4,4′,4′,5,5,5′,5′-octamethyl-2,2′-bi(1,3,2-dioxaborolane) (448 mg, 1.76 mmol), KOAc (346 mg, 3.53 mmol) and Pd(dppf)Cl₂ (86 mg, 118 μmol) in 1,4-dioxane (6 mL) was degassed and purged with N₂ three times. The mixture was stirred at 90° C. for 3 hr under N₂ atmosphere. (2-(2-Aminopyridin-3-yl)-3-(4-(((tert-butyldimethylsilyl)oxy)methyl)phenyl)-3H-imidazo[4,5-b]pyridin-5-yl)boronic acid was obtained as a black liquid, which was used in the next step without work-up and purification. MS: m/z=476.2 [M+H]⁺.

### Step 2: 3-(3-(4-(((tert-Butyldimethylsilyl)oxy)methyl)phenyl)-5-(6-(methoxy-d₃)pyridin-3-yl-2-d)-3H-imidazo[4,5-b]pyridin-2-yl)pyridin-2-amine

A mixture of (2-(2-aminopyridin-3-yl)-3-(4-(((tert-butyldimethylsilyl)oxy)methyl)phenyl)-3H-imidazo[4,5-b]pyridin-5-yl)boronic acid (559 mg, 1.18 mmol), Intermediate 264 (225 mg, 1.18 mmol), Cs₂CO₃ (1.15 g, 3.53 mmol) and Pd(dppf)Cl₂ (86 mg, 118 μmol) in 1,4-dioxane (7 mL) and H₂O (1.5 mL) was degassed and purged with N₂ three times. The mixture was stirred at 80° C. for 16 hr under N₂ atmosphere. The reaction mixture was quenched with H₂O (20 mL) and extracted with EtOAc (15 mL×2). The combined organic layers were washed with brine (30 mL), dried over Na₂SO₄, filtered and concentrated under reduced pressure. After purified by silica gel flash chromatography (Eluent of 20%˜41% EtOAc in petroleum ether), 3-(3-(4-(((tert-butyldimethylsilyl)oxy)methyl)phenyl)-5-(6-(methoxy-d₃)pyridin-3-yl-2-d)-3H-imidazo[4,5-b]pyridin-2-yl)pyridin-2-amine (350 mg, yield. 49% for two steps) was obtained as a black solid. MS: m/z=542.3 [M+H]⁺. D %: 3D %=68.2%, 4D %=30.4%. ¹H NMR (400 MHz, Chloroform-d) δ 8.84 (s, 1H), 8.27-8.01 (m, 3H), 7.71 (d, J=8.4 Hz, 1H), 7.48 (d, J=8.0 Hz, 2H), 7.38 (d, J=8.4 Hz, 2H), 7.16-7.12 (m, 1H), 6.82-6.75 (m, 1H), 6.65 (br s, 2H), 6.37 (dd, J=8.0, 5.2 Hz, 1H), 4.86 (s, 2H), 0.98 (s, 9H), 0.15 (s, 6H).

### Step 3: (4-(2-(2-Aminopyridin-3-yl)-5-(6-(methoxy-d₃)pyridin-3-yl-2-d)-3H-imidazo[4,5-b]pyridin-3-yl)phenyl)methanol

To a solution of 3-(3-(4-(((tert-butyldimethylsilyl)oxy)methyl)phenyl)-5-(6-(methoxy-d₃)pyridin-3-yl-2-d)-3H-imidazo[4,5-b]pyridin-2-yl)pyridin-2-amine (350 mg, 646 μmol) in THE (3 mL) was added TBAF (507 mg, 1.94 mmol, 1 M in THF). The mixture was stirred at 25° C. for 1 hr. The reaction mixture was quenched with H₂O (20 mL) at 25° C. and extracted with EtOAc (15 mL×2). The combined organic layers were washed with brine (50 mL×3), dried over Na₂SO₄, filtered and concentrated under reduced pressure. After triturated with EtOAc (10 mL) at 25° C. for 10 min, (4-(2-(2-aminopyridin-3-yl)-5-(6-(methoxy-d₃)pyridin-3-yl-2-d)-3H-imidazo[4,5-b]pyridin-3-yl)phenyl)methanol (200 mg, yield: 69%) was obtained as a black solid. MS: m/z=428.2 [M+H]⁺. D %: 3D %=66.4%, 4D %=31.9%. ¹H NMR (400 MHz, Chloroform-d) δ 8.83 (d, J=0.8 Hz, 1H), 8.21 (dd, J=8.4, 2.4 Hz, 1H), 8.12 (d, J=8.4 Hz, 1H), 8.07 (dd, =4.8, 1.6 Hz, 1H), 7.71 (d, J=8.4 Hz, 1H), 7.56-7.52 (m, 2H), 7.45-7.40 (m, 2H), 7.14 (dd, J=8.0, 1.6 Hz, 1H), 6.90-6.70 (m, 1H), 6.62 (br s, 2H), 6.39 (dd, J=7.6, 4.8 Hz, 1H), 4.83 (s, 2H).

### Step 4: 3-(3-(4-(Chloromethyl)phenyl)-5-(6-(methoxy-d₃)pyridin-3-yl-2-d)-3H-imidazo[4,5-b]pyridin-2-yl)pyridin-2-amine

To a solution of (4-(2-(2-aminopyridin-3-yl)-5-(6-(methoxy-d₃)pyridin-3-yl-2-d)-3H-imidazo[4,5-b]pyridin-3-yl)phenyl)methanol (200 mg, 468 μmol) in CH₂Cl₂ (5 mL) was added SOCl₂ (167 mg, 1.40 mmol). The mixture was stirred at 25° C. for 1 hr. The reaction was concentrated under reduced pressure to give 3-(3-(4-(chloromethyl)phenyl)-5-(6-(methoxy-d₃)pyridin-3-yl-2-d)-3H-imidazo[4,5-b]pyridin-2-yl)pyridin-2-amine (Intermediate 265, 226 mg, HCl salt) was obtained as a brown solid. MS: m/z=446.1 [M+H]⁺. D %: 3D %=66.8%, 4D %=32.9%. ¹H NMR (400 MHz, Methanol-d₄) δ 9.07 (dd, J=9.2, 2.4 Hz, 1H), 8.97 (s, 1H), 8.42 (d, J=8.4 Hz, 1H), 8.16 (d, J=8.4 Hz, JH), 8.03 (dd, J=6.0, 1.2 Hz, 1H), 7.87 (dd, J=7.6, 1.6 Hz, 1H), 7.71 (d, J=8.4 Hz, 2H), 7.63-7.58 (m, 3H), 6.87 (dd, J=7.6, 6.4 Hz, 1H), 4.79 (s, 2H).

### Intermediate 266: 5-Bromo-2-(methoxy-d₃)pyridine

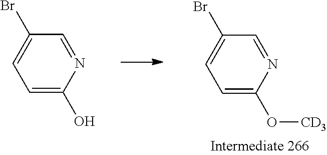 [SMILES: Oc1ccc(Br)cn1 ; [2H]C([2H])([2H])Oc1ccc(Br)cn1]

To a solution of 5-bromopyridin-2-ol (1 g, 5.75 mmol) and Ag₂CO₃ (1.11, 4.02 mmol) in CH₂Cl₂ (10 mL) was added CD₃I (979 mg, 6.9 mmol). The mixture was stirred at 50° C. for 16 hr. The reaction mixture was quenched with H₂O (50 mL) at 25° C. and extracted with CH₂Cl₂ (50 mL×3). The combined organic layers were washed with brine (50 mL×3), dried over Na₂SO₄, filtered and concentrated under reduced pressure. After purified by silica gel flash chromatography (Eluent of 0%˜5% EtOAc in petroleum ether), 5-bromo-2-(methoxy-d₃)pyridine (Intermediate 266, 480 mg, yield: 43%) was obtained as a colorless oil. MS: m/z=190.9, 192.9 [M+H]⁺. 3D %=100%. ¹H NMR (400 MHz, Dimethylsulfoxide-d₆) 58.29 (d, J=2.4 Hz, 1H), 7.89 (dd, J=8.8, 2.4 Hz, 1H), 6.84 (d, J=8.8 Hz, 1H).

### Intermediate 267: 3-(3-(4-(Chloromethyl)phenyl)-5-(6-(methoxy-d₃)pyridin-3-yl)-3H-imidazo[4,5-b]pyridin-2-yl)pyridin-2-amine

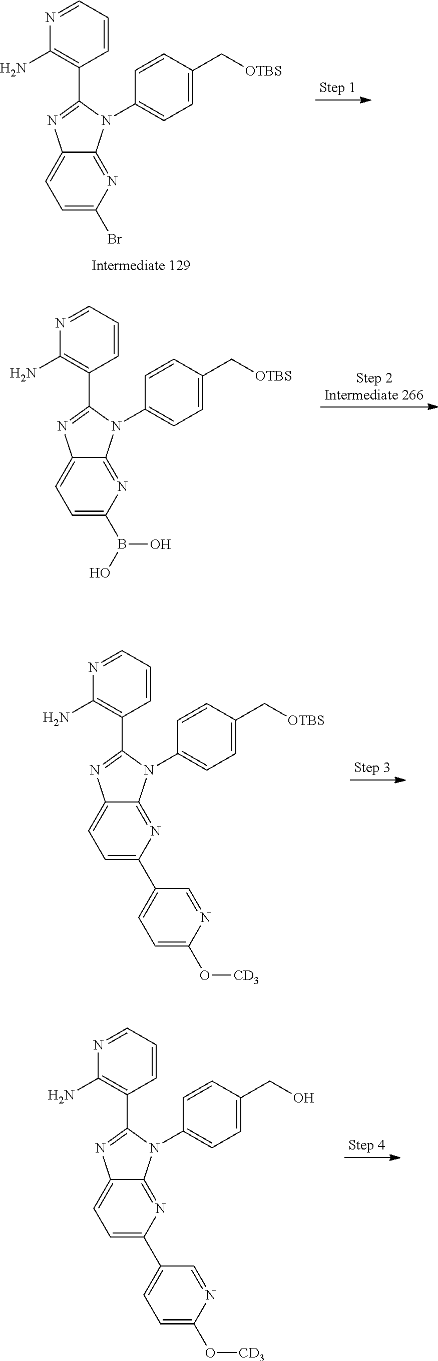 [SMILES: CC(C)(C)[Si](C)(C)OCc1ccc(-n2c(-c3cccnc3N)nc3ccc(Br)nc32)cc1 ; CC(C)(C)[Si](C)(C)OCc1ccc(-n2c(-c3cccnc3N)nc3ccc(B(O)O)nc32)cc1 ; [2H]C([2H])([2H])Oc1ccc(-c2ccc3nc(-c4cccnc4N)n(-c4ccc(CO[Si](C)(C)C(C)(C)C)cc4)c3n2)cn1 ; [2H]C([2H])([2H])Oc1ccc(-c2ccc3nc(-c4cccnc4N)n(-c4ccc(CO)cc4)c3n2)cn1]

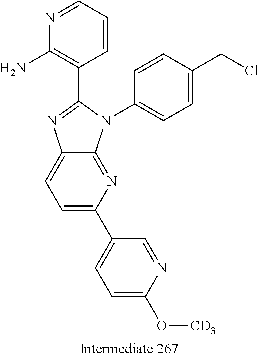 [SMILES: [2H]C([2H])([2H])Oc1ccc(-c2ccc3nc(-c4cccnc4N)n(-c4ccc(CCl)cc4)c3n2)cn1]

### Step 1: (2-(2-Aminopyridin-3-yl)-3-(4-(((tert-butyldimethylsilyl)oxy)methyl)phenyl)-3H-imidazo[4,5-b]pyridin-5-yl)boronic acid

A mixture of Intermediate 129 (1 g, 1.96 mmol), 4,4,4′,4′,5,5,5′,5′-octamethyl-2,2′-bi(1,3,2-dioxaborolane) (995 mg, 3.92 mmol), KOAc (577 mg, 5.88 mmol) and Pd(dppf)Cl₂ (143 mg, 196 μmol) in 1,4-dioxane (10 mL) was degassed and purged with N₂ three times. The mixture was stirred at 80° C. for 4 hr under N₂ atmosphere. (2-(2-Aminopyridin-3-yl)-3-(4-(((tert-butyldimethylsilyl)oxy)methyl)phenyl)-3H-imidazo[4,5-b]pyridin-5-yl)boronic acid was obtained as a black liquid, which was used in the next step without work-up and purification. MS: m/z=476.2 [M+H]⁺.

### Step 2: 3-(3-(4-(((tert-Butyldimethylsilyl)oxy)methyl)phenyl)-5-(6-(methoxy-d₃)pyridin-3-yl)-3H-imidazo[4,5-b]pyridin-2-yl)pyridin-2-amine

A mixture of (2-(2-aminopyridin-3-yl)-3-(4-(((tert-butyldimethylsilyl)oxy)methyl)phenyl)-3H-imidazo[4,5-b]pyridin-5-yl)boronic acid (930 mg, 1.96 mmol), Intermediate 266 (374 mg, 1.96 mmol), Pd(dppf)Cl₂ (143 mg, 196 μmol) and Cs₂CO₃ (1.91 g, 5.87 mmol) in 1,4-dioxane (10 mL) and H₂O (2 mL) was degassed and purged with N₂ three times. The mixture was stirred at 80° C. for 2 hr under N₂ atmosphere. The reaction mixture was quenched with H₂O (50 mL) and extracted with CH₂Cl₂ (50 mL×2). The combined organic layers were washed with brine (50 mL), dried over Na₂SO₄, filtered and concentrated under reduced pressure. After purified by silica gel flash chromatography (Eluent of 10%˜40% EtOAc in petroleum ether), 3-(3-(4-(((tert-butyldimethylsilyl)oxy)methyl)phenyl)-5-(6-(methoxy-d₃)pyridin-3-yl)-3H-imidazo[4,5-b]pyridin-2-yl)pyridin-2-amine (440 mg, yield: 39% for two steps) was obtained as a yellow solid. MS: m/z=542.4 [M+H]⁺. ¹H NMR (400 MHz, Dimethylsulfoxide-d₆) δ 8.84 (d, J=2.4 Hz, 1H), 8.32-8.19 (m, 2H), 8.03-7.93 (m, 2H), 7.53-7.43 (m, 4H), 7.20-7.17 (m, 1H), 7.03 (s, 2H), 6.89 (d, J=8.8 Hz, 1H), 6.38 (dd, J=7.6, 4.8 Hz, 1H), 4.82 (s, 2H), 0.92 (s, 9H), 0.10 (s, 6H).

### Step 3: (4-(2-(2-Aminopyridin-3-yl)-5-(6-(methoxy-d₃)pyridin-3-yl)-3H-imidazo[4,5-b]pyridin-3-yl)phenyl)methanol

To a solution of 3-(3-(4-(((tert-butyldimethylsilyl)oxy)methyl)phenyl)-5-(6-(methoxy-d₃)pyridin-3-yl)-3H-imidazo[4,5-b]pyridin-2-yl)pyridin-2-amine (440 mg, 812 μmol) in THF (5 mL) was added TBAF (425 mg, 1.62 mmol, 1 M in THF). The mixture was stirred at 25° C. for 1 hr. The reaction mixture was quenched with H₂O (20 mL) at 25° C. and extracted with CH₂Cl₂ (20 mL×3). The combined organic layers were washed with brine (20 mL×3), dried over Na₂SO₄, filtered and concentrated under reduced pressure. (4-(2-(2-Aminopyridin-3-yl)-5-(6-(methoxy-d₃)pyridin-3-yl)-3H-imidazo[4,5-b]pyridin-3-yl)phenyl)methanol 340 mg, yield: 98%) was obtained as a brown solid. MS: m/z=428.2 [M+H]⁺. ¹H NMR (400 MHz, Dimethylsulfoxide-d₆) δ 8.84 (d, J=2.4 Hz, 1H), 8.33-8.20 (m, 2H), 8.02-7.94 (m, 2H), 7.53-7.41 (m, 4H), 7.21 (dd, J=7.6, 1.6 Hz, 1H), 7.00 (s, 2H), 6.90 (d, J=8.8 Hz, 1H), 6.41 (dd, J=7.6, 4.8 Hz, 1H), 5.36 (t, J=5.6 Hz, 1H), 4.60 (d, J=5.6 Hz, 2H).

### Step 4: 3-(3-(4-(Chloromethyl)phenyl)-5-(6-(methoxy-d₃)pyridin-3-yl)-3H-imidazo[4,5-b]pyridin-2-yl)pyridin-2-amine

To a solution of (4-(2-(2-aminopyridin-3-yl)-5-(6-(methoxy-d₃)pyridin-3-yl)-3H-imidazo[4,5-b]pyridin-3-yl)phenyl)methanol (340 mg, 795 μmol) in CH₂Cl₂ (5 mL) was added SOCl₂ (284 mg, 2.39 mmol). The mixture was stirred at 40° C. for 0.5 hr. The reaction was concentrated under reduced pressure to give 3-(3-(4-(chloromethyl)phenyl)-5-(6-(methoxy-d₃)pyridin-3-yl)-3H-imidazo[4,5-b]pyridin-2-yl)pyridin-2-amine (Intermediate 267, 380 mg, HCl salt) was obtained as a brown solid. MS: m/z=446.1 [M+H]⁺.

### Intermediate 268: 2-(2-Aminopyridin-3-yl)-3-(4-(bromomethyl)phenyl)-5-hydroxy-3H-imidazo[4,5-b]pyridine-6-carbonitrile

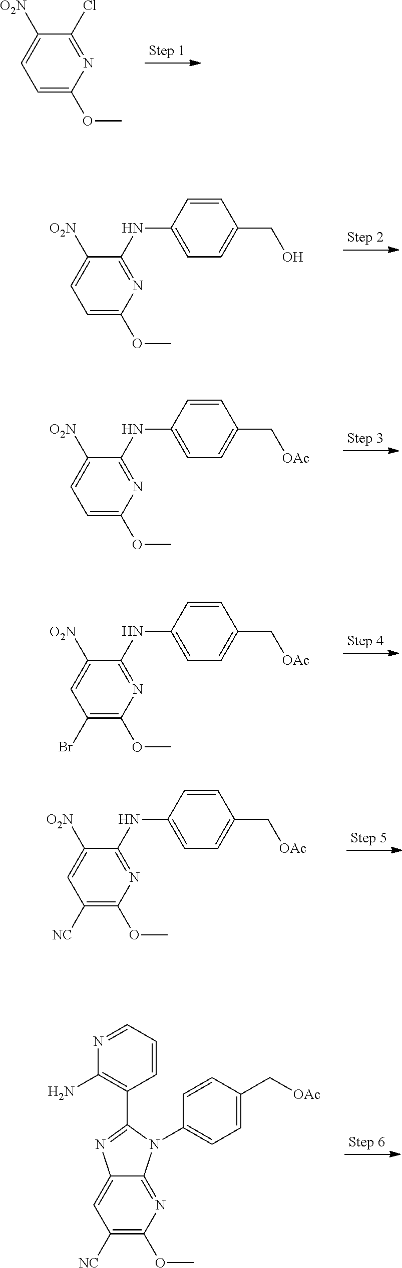

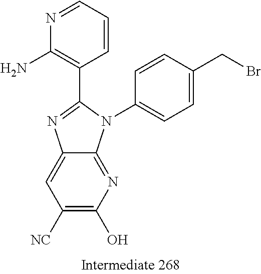 [SMILES: N#Cc1cc2nc(-c3cccnc3N)n(-c3ccc(CBr)cc3)c2nc1O]

### Step 1: (4-((6-Methoxy-3-nitropyridin-2-yl)amino)phenyl)methanol

To a solution of 2-chloro-6-methoxy-3-nitropyridine (12 g, 64 mmol), and DIEA (25 g, 191 mmol) in DMSO (100 mL) was added (4-aminophenyl)methanol (7.8 g, 64 mmol) at 25° C. The mixture was stirred at 80° C. for 12 hr. The mixture was quenched with H₂O (300 mL) and extracted with CH₂Cl₂ (100 mL×2). The combined organic layers were washed with brine (200 mL×3), dried over Na₂SO₄, filtered and concentrated. (4-((6-Methoxy-3-nitropyridin-2-yl)amino)phenyl)methanol (15 g, yield: 79%) was obtained as a yellow solid. MS: m/z=275.8 [M+H]⁺. ¹H NMR (400 MHz, Dimethylsulfoxide-d₆) δ 10.45 (s, 1H), 8.43 (d, J=9.2 Hz, 1H), 7.66 (d, J=8.4 Hz, 2H), 7.33 (d, J=8.4 Hz, 2H), 6.36 (d, J=9.2 Hz, 1H), 5.18 (t, 1=5.6 Hz, 1H), 4.49 (d, J=5.6 Hz, 2H), 3.88 (s, 3H).

### Step 2: 4-((6-Methoxy-3-nitropyridin-2-yl)amino)benzyl acetate

To a solution of (4-((6-methoxy-3-nitropyridin-2-yl)amino)phenyl)methanol (15 g, 54 mmol), TEA (16 g, 163 mmol), and DMAP (666 g, 5.4 mmol) in CH₂Cl₂ (100 mL) was added acetic anhydride (6.7 g, 65 mmol) at 0° C. The mixture was stirred at 0° C. for 2 hr. The mixture was quenched with H₂O (300 mL) and extracted with CH₂Cl₂ (100 mL×2). The combined organic layers were washed with brine (200 mL×3), dried over Na₂SO₄, filtered and concentrated. After purified by silica gel flash chromatography (Eluent of 0%˜40% EtOAc in petroleum ether), 4-((6-methoxy-3-nitropyridin-2-yl)amino)benzyl acetate (16.5 g, yield: 88%) was obtained as a yellow solid. MS: m/z=318.0 [M+H]⁺. ¹H NMR (400 MHz, Dimethylsulfoxide-d₆) δ 10.47 (s, 1H), 8.44 (d, J=9.2 Hz, 1H), 7.73 (d, J=8.8 Hz, 2H), 7.39 (d, J=8.4 Hz, 2H), 6.38 (d, J=9.2 Hz, 1H), 5.06 (s, 2H), 3.89 (s, 3H), 2.07 (s, 3H).

### Step 3: 4-((5-Bromo-6-methoxy-3-nitropyridin-2-yl)amino)benzyl acetate

To a solution of 4-((6-methoxy-3-nitropyridin-2-yl)amino)benzyl acetate (12 g, 38 mmol) in DMF (50 mL) was added NBS (8.0 g, 45 mmol) at 25° C. The mixture was stirred at 90° C. for 2 hr. The mixture was quenched with H₂O (300 mL) and extracted with CH₂Cl₂ (100 mL×2). The combined organic layers were washed with brine (200 mL×3), dried over Na₂SO₄, filtered and concentrated. 4-((5-Bromo-6-methoxy-3-nitropyridin-2-yl)amino)benzyl acetate (14 g, crude) was obtained as a yellow solid. MS: m/z=396.0, 397.8[M+H]+. ¹H NMR (400 MHz, Dimethylsulfoxide-d₆) δ 10.37 (s, 1H), 8.65 (s, 1H), 7.70 (d, J=8.4 Hz, 2H), 7.39 (d, J=8.8 Hz, 2H), 5.07 (s, 2H), 3.94 (s, 3H), 2.07 (s, 3H).

### Step 4: 4-((5-Cyano-6-methoxy-3-nitropyridin-2-yl)amino)benzyl acetate

A mixture of 4-((5-bromo-6-methoxy-3-nitropyridin-2-yl)amino)benzyl acetate (5 g, 12.6 μmol) and CuCN (5.6 g, 63 mmol) in DMF (20 mL) was stirred at 180° C. for 2 hr. The mixture was quenched with H₂O (200 mL) and extracted with CH₂Cl₂ (100 mL×2). The combined organic layers were washed with brine (200 mL×3), dried over Na₂SO₄, filtered and concentrated. After purified by silica gel flash chromatography (Eluent of 0%˜15% EtOAc in petroleum ether), 4-((5-cyano-6-methoxy-3-nitropyridin-2-yl)amino)benzyl acetate (3.3 g, yield: 69% for two steps) was obtained as a yellow solid. ¹H NMR (400 MHz, Dimethylsulfoxide-d₆) δ 10.61 (s, 1H), 8.99 (s, 1H), 7.68 (d, J=8.0 Hz, 2H), 7.41 (d, J=8.4 Hz, 2H), 5.08 (s, 2H), 3.94 (s, 3H), 2.07 (s, 3H).

### Step 5: 4-(2-(2-Aminopyridin-3-yl)-6-cyano-5-methoxy-3H-imidazo[4,5-b]pyridin-3-yl)benzyl acetate

To a solution of 4-((5-cyano-6-methoxy-3-nitropyridin-2-yl)amino)benzyl acetate (1.2 g, 3.5 mmol) in DMSO (50 mL) were added 2-aminonicotinaldehyde (514 mg, 4.2 mmol) and Na₂S₂O₄ (2.4 g, 14 mmol) at 25° C. The mixture was stirred at 100° C. for 12 hr. The reaction mixture was poured into H₂O (200 mL) and extracted with CH₂Cl₂ (50 mL×2). The combined organic layers were washed with brine (200 mL), dried over anhydrous Na₂SO₄, filtered and concentrated under reduced pressure. After purified by silica gel flash chromatography (Eluent of 0%˜25% EtOAc in petroleum ether), 4-(2-(2-aminopyridin-3-yl)-6-cyano-5-methoxy-3H-imidazo[4,5-b]pyridin-3-yl)benzyl acetate (400 mg, yield: 24%) was obtained as a yellow solid. MS: m/z=414.9 [M+H]⁺. ¹H NMR (400 MHz, Dimethylsulfoxide-d₆) δ 8.74 (s, 1H), 8.00 (dd, J=4.8, 2.0 Hz, 1H), 7.53-7.44 (m, 4H), 7.20 (dd, J=7.6, 2.0 Hz, 1H), 6.72 (s, 2H), 6.43 (dd, J=7.6, 4.8 Hz, 1H), 5.15 (s, 2H), 3.90 (s, 3H), 2.10 (s, 3H).

### Step 6: 2-(2-Aminopyridin-3-yl)-3-(4-(bromomethyl)phenyl)-5-hydroxy-3H-imidazo[4,5-b]pyridine-6-carbonitrile

To a solution of 4-(2-(2-aminopyridin-3-yl)-6-cyano-5-methoxy-3H-imidazo[4,5-b]pyridin-3-yl)benzyl acetate (400 mg, 965 μmol) in DCE (10 mL) was added BBr₃ (4.8 mL, 4.8 mmol, 1 M in CH₂Cl₂). The mixture was stirred at 80° C. for 12 hr. The reaction mixture was quenched with H₂O (20 mL) at 25° C. The pH of the mixture was adjusted to 8 with saturated aq. NaHCO₃. The mixture was extracted with CH₂Cl₂ (15 mL×2). The combined organic layers were washed with brine (30 mL×2), dried over Na₂SO₄, filtered and concentrated under reduced pressure. 2-(2-Aminopyridin-3-yl)-3-(4-(bromomethyl)phenyl)-5-hydroxy-3H-imidazo[4,5-b]pyridine-6-carbonitrile (Intermediate 268, 350 mg, crude) was obtained as a yellow solid. MS: m/z=420.8, 422.7 [M+H]⁺.

### Intermediate 269: 2-Amino-4-fluoronicotinaldehyde

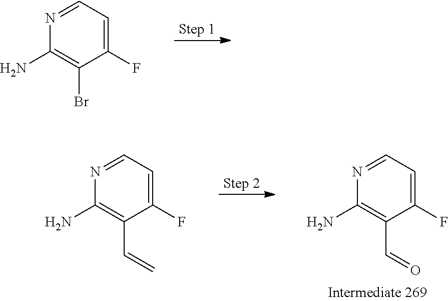

### Step 1: 4-Fluoro-3-vinylpyridin-2-amine

A mixture of 3-bromo-4-fluoropyridin-2-amine (1 g, 5.24 mmol), 4,4,5,5-tetramethyl-2-vinyl-1,3,2-dioxaborolane (1.21 g, 7.85 mmol), Cs₂CO₃ (5.12 g, 15.7 mmol) and Pd(dppf)Cl₂ (383 mg, 524 μmol) in 1,4-dioxane (20 mL) and H₂O (4 mL) was degassed and purged with N₂ three times. The mixture was stirred at 80° C. for 16 hr under N₂ atmosphere. The reaction mixture was quenched with H₂O (50 mL) at 25° C. and extracted with EtOAc (50 mL×2). The combined organic layers were washed with brine (50 mL), dried over anhydrous Na₂SO₄, filtered and concentrated under reduced pressure. After purified by silica gel flash chromatography (Eluent of 0%˜15% EtOAc in petroleum ether), 4-fluoro-3-vinylpyridin-2-amine (700 mg, yield: 77%) was obtained as a light-yellow oil. MS: m/z=139.0 [M+H]⁺. ¹H NMR (400 MHz, Chloroform-d) δ 7.94-7.88 (m, 1H), 6.56-6.42 (m, 2H), 5.78 (d, J=17.6 Hz, 1H), 5.63 (d, J=11.6 Hz, 1H), 4.80 (br s, 2H). ¹⁹F NMR (400 MHz, Chloroform-d) δ −106.82.

### Step 2: 2-Amino-4-fluoronicotinaldehyde

To a solution of 4-fluoro-3-vinylpyridin-2-amine (700 mg, 5.07 mmol) in H₂O (5 mL) and THF (15 mL) were added NaIO₄ (3.25 g, 15.2 mmol) and K₂OsO₄·2H₂O (560 mg, 1.52 mmol). The mixture was stirred at 25° C. for 1 hr. The reaction mixture was quenched with aqueous Na₂SO₃ (50 mL) at 25° C. and extracted with CH₂Cl₂ (30 mL×2). The combined organic layers were washed with brine (50 mL), dried over anhydrous Na₂SO₄, filtered and concentrated under reduced pressure. After purified by silica gel flash chromatography (Eluent of 5%˜13% EtOAc in petroleum ether), 2-amino-4-fluoronicotinaldehyde (Intermediate 269, 275 mg, yield: 38%) was obtained as a white solid. MS: m/z=141.0 [M+H]⁺. ¹H NMR (400 MHz, Dimethylsulfoxide-d₆) δ 10.19 (s, 1H), 8.26 (dd, J=8.8, 5.6 Hz, 1H), 7.96 (br s, 2H), 6.56 (dd, J=10.8, 5.6 Hz, 1H).

### Intermediate 270: 3-(3-(4-(Chloromethyl)phenyl)-5-(5-fluoropyridin-2-yl)-3H-imidazo[4,5-b]pyridin-2-yl)-4-fluoropyridin-2-amine

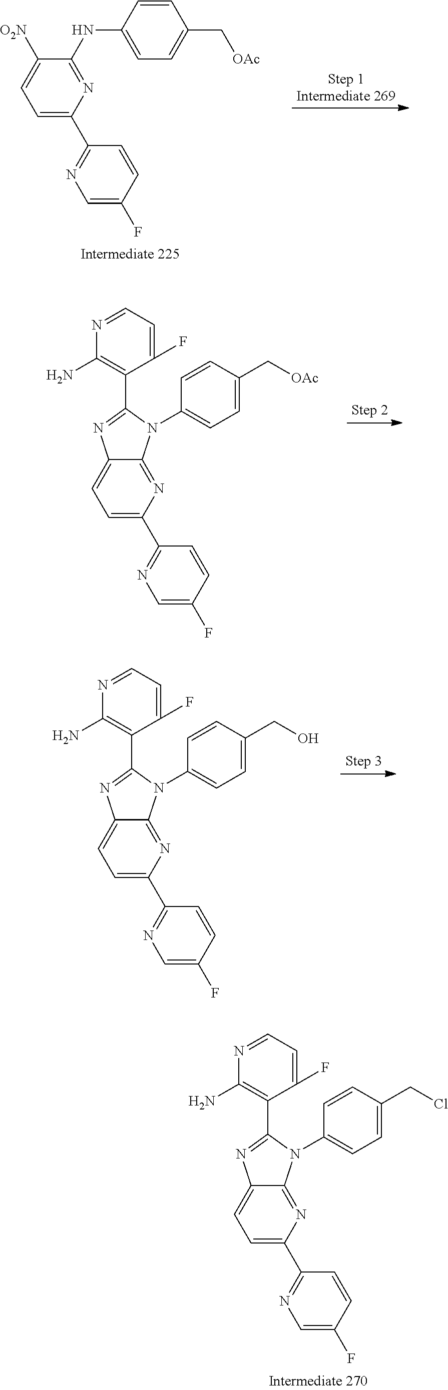 [SMILES: CC(=O)OCc1ccc(Nc2nc(-c3ccc(F)cn3)ccc2[N+](=O)[O-])cc1 ; CC(=O)OCc1ccc(-n2c(-c3c(F)ccnc3N)nc3ccc(-c4ccc(F)cn4)nc32)cc1 ; Nc1nccc(F)c1-c1nc2ccc(-c3ccc(F)cn3)nc2n1-c1ccc(CO)cc1 ; Nc1nccc(F)c1-c1nc2ccc(-c3ccc(F)cn3)nc2n1-c1ccc(CCl)cc1]

### Step 1: 4-(2-(2-Amino-4-fluoropyridin-3-yl)-5-(5-fluoropyridin-2-yl)-3H-imidazo[4,5-b]pyridin-3-yl)benzyl acetate

To a solution of Intermediate 269 (200 mg, 1.43 mmol) in DMSO (10 mL) were added Na₂S₂O₄ (1.03 g, 5.19 mmol, 88% purity) and Intermediate 225 (496 mg, 1.30 mmol). The mixture was stirred at 60° C. for 16 hr. The reaction mixture was quenched with H₂O (50 mL) and extracted with CH₂Cl₂ (15 mL×2). The combined organic layers were washed with brine (50 mL×3), dried over Na₂SO₄, filtered and concentrated under reduced pressure. After purified by prep-HPLC (column: Phenomenex C18 150×25 mm×10 μm; mobile phase: [water (NH₄HCO₃)-ACN]; gradient: 32%-62% B over 11 min), 4-(2-(2-amino-4-fluoropyridin-3-yl)-5-(5-fluoropyridin-2-yl)-3H-imidazo[4,5-b]pyridin-3-yl)benzyl acetate (20 mg, yield: 1.3%) was obtained as a yellow solid. MS: m/z=495.1 [M+Na]⁺. ¹H NMR (400 MHz, Chloroform-d) δ 8.51-8.50 (m, 1H), 8.44-8.41 (m, 2H), 8.24-8.20 (m, 2H), 8.09 (dd, J=8.4, 5.6 Hz, 1H), 7.87-7.83 (m, 1H), 7.49-7.45 (m, 3H), 7.32 (br s, 2H), 7.14 (d, J=8.0 Hz, 1H), 5.20 (s, 2H), 2.16 (s, 3H).

### Step 2: (4-(2-(2-Amino-4-fluoropyridin-3-yl)-5-(5-fluoropyridin-2-yl)-3H-imidazo[4,5-b]pyridin-3-yl)phenyl)methanol

To a solution of 4-(2-(2-amino-4-fluoropyridin-3-yl)-5-(5-fluoropyridin-2-yl)-3H-imidazo[4,5-b]pyridin-3-yl)benzyl acetate (20 mg, 42.3 μmol) in THF (1 mL), MeOH (1 mL) and H₂O (1 mL) was added K₂CO₃ (17.6 mg, 127 μmol). The mixture was stirred at 25° C. for 1 hr. The reaction mixture was quenched with H₂O (5 mL) at 25° C. and extracted with CH₂Cl₂ (15 mL×2). The combined organic layers were washed with brine (30 mL), dried over Na₂SO₄, filtered and concentrated under reduced pressure. (4-(2-(2-Amino-4-fluoropyridin-3-yl)-5-(5-fluoropyridin-2-yl)-3H-imidazo[4,5-b]pyridin-3-yl)phenyl)methanol (18 mg) was obtained as a yellow solid. MS: m/z=431.1 [M+H]⁺.

### Step 3: 3-(3-(4-(Chloromethyl)phenyl)-5-(5-fluoropyridin-2-yl)-3H-imidazo[4,5-b]pyridin-2-yl)-4-fluoropyridin-2-amine

To a solution of (4-(2-(2-amino-4-fluoropyridin-3-yl)-5-(5-fluoropyridin-2-yl)-3H-imidazo[4,5-b]pyridin-3-yl)phenyl)methanol (18 mg, 41.8 μmol) in CH₂Cl₂ (3 mL) was added SOCl₂ (14.9 mg, 125 μmol). The mixture was stirred at 25° C. for 0.5 hr. The reaction was concentrated under reduced pressure to give 3-(3-(4-(chloromethyl)phenyl)-5-(5-fluoropyridin-2-yl)-3H-imidazo[4,5-b]pyridin-2-yl)-4-fluoropyridin-2-amine (Intermediate 270, 20 mg, HCl salt) as a yellow solid. MS: m/z=449.0 [M+H]⁺.

### Intermediate 271: 3-(4-(Chloromethyl)phenyl)-5-(5-fluoropyridin-2-yl)-2-(pyrimidin-5-yl)-3H-imidazo[4,5-b]pyridine

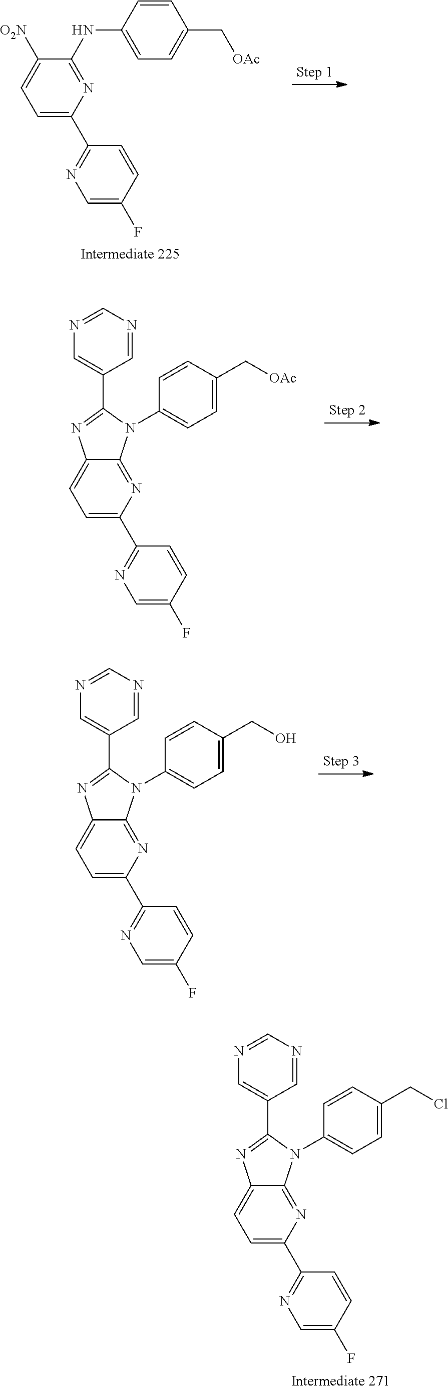 [SMILES: CC(=O)OCc1ccc(Nc2nc(-c3ccc(F)cn3)ccc2[N+](=O)[O-])cc1 ; CC(=O)OCc1ccc(-n2c(-c3cncnc3)nc3ccc(-c4ccc(F)cn4)nc32)cc1 ; OCc1ccc(-n2c(-c3cncnc3)nc3ccc(-c4ccc(F)cn4)nc32)cc1 ; Fc1ccc(-c2ccc3nc(-c4cncnc4)n(-c4ccc(CCl)cc4)c3n2)nc1]

### Step 1: 4-(5-(5-Fluoropyridin-2-yl)-2-(pyrimidin-5-yl)-3H-imidazo[4,5-b]pyridin-3-yl)benzyl acetate

To a solution of Intermediate 225 (1 g, 2.62 mmol) in DMSO (15 mL) were added Na₂S₂O₄ (1.82 g, 10.46 mmol, 88% purity) and pyrimidine-5-carbaldehyde (339 mg, 3.14 mmol). The mixture was stirred at 100° C. for 16 hr. The reaction mixture was quenched with H₂O (100 mL) at 25° C., diluted with EtOAc (100 mL), and extracted with EtOAc (100 mL×2). The combined organic layers were washed with brine (100 mL), dried over Na₂SO₄, filtered and concentrated under reduced pressure. After purified by silica gel flash chromatography (Eluent of 0%˜35% EtOAc in petroleum ether), 4-(5-(5-fluoropyridin-2-yl)-2-(pyrimidin-5-yl)-3H-imidazo[4,5-b]pyridin-3-yl)benzyl acetate (550 mg, yield: 28%) was obtained as a light yellow solid. MS: m/z=441.1 [M+H]⁺.

### Step 2: (4-(5-(5-Fluoropyridin-2-yl)-2-(pyrimidin-5-yl)-3H-imidazo[4,5-b]pyridin-3-yl)phenyl)methanol

To a solution of 4-(5-(5-fluoropyridin-2-yl)-2-(pyrimidin-5-yl)-3H-imidazo[4,5-b]pyridin-3-yl)benzyl acetate (550 mg, 1.25 mmol) in THF (4 mL), MeOH (4 mL) and H₂O (2 mL) was added K₂CO₃ (518 mg, 3.75 mmol). The mixture was stirred at 25° C. for 1 hr. The reaction mixture was diluted with H₂O (100 mL) and extracted with EtOAc (100 mL×2). The combined organic layers were washed with brine (100 mL), dried over Na₂SO₄, filtered and concentrated under reduced pressure. (4-(5-(5-Fluoropyridin-2-yl)-2-(pyrimidin-5-yl)-3H-imidazo[4,5-b]pyridin-3-yl)phenyl)methanol (490 mg) was obtained as a light yellow solid. MS: m/z=399.1 [M+H]⁺.

### Step 3: 3-(4-(Chloromethyl)phenyl)-5-(5-fluoropyridin-2-yl)-2-(pyrimidin-5-yl)-3H-imidazo[4,5-b]pyridine

To a solution of (4-(5-(5-fluoropyridin-2-yl)-2-(pyrimidin-5-yl)-3H-imidazo[4,5-b]pyridin-3-yl)phenyl)methanol (490 mg, 1.23 mmol) in CH₂Cl₂ (3 mL) was added SOCl₂ (439 mg, 3.69 mmol). The mixture was stirred at 40° C. for 0.5 hr. The reaction was concentrated under reduced pressure to give 3-(4-(chloromethyl)phenyl)-5-(5-fluoropyridin-2-yl)-2-(pyrimidin-5-yl)-3H-imidazo[4,5-b]pyridine (Intermediate 271, 450 mg, HCl salt) was obtained as a light yellow solid. MS: m/z=417.1, 419.1 [M+H]⁺.

### Intermediate 272: 3-(1-(4-(Chloromethyl)phenyl)-6-(5-fluoropyridin-2-yl)-1H-benzo[d]imidazol-2-yl)pyridin-2-amine

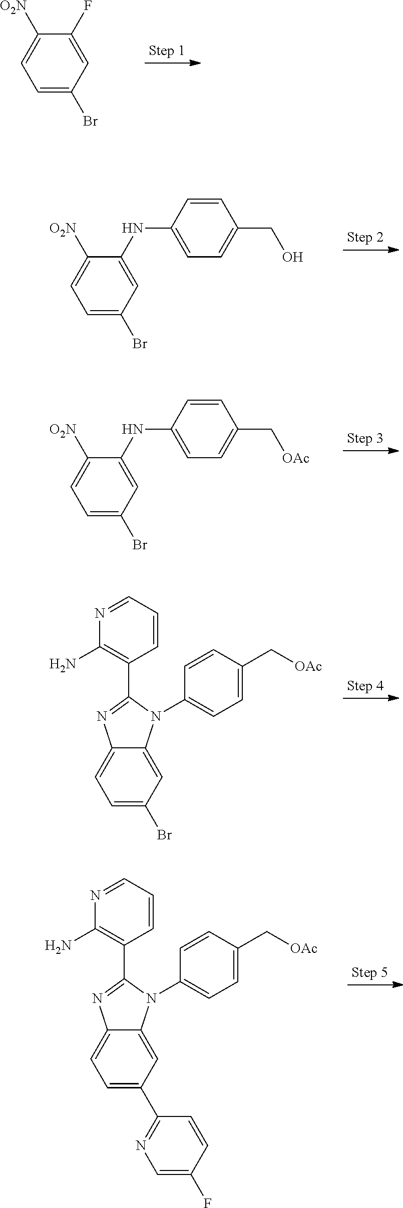 [SMILES: O=[N+]([O-])c1ccc(Br)cc1F ; O=[N+]([O-])c1ccc(Br)cc1Nc1ccc(CO)cc1 ; CC(=O)OCc1ccc(Nc2cc(Br)ccc2[N+](=O)[O-])cc1 ; CC(=O)OCc1ccc(-n2c(-c3cccnc3N)nc3ccc(Br)cc32)cc1 ; CC(=O)OCc1ccc(-n2c(-c3cccnc3N)nc3ccc(-c4ccc(F)cn4)cc32)cc1]

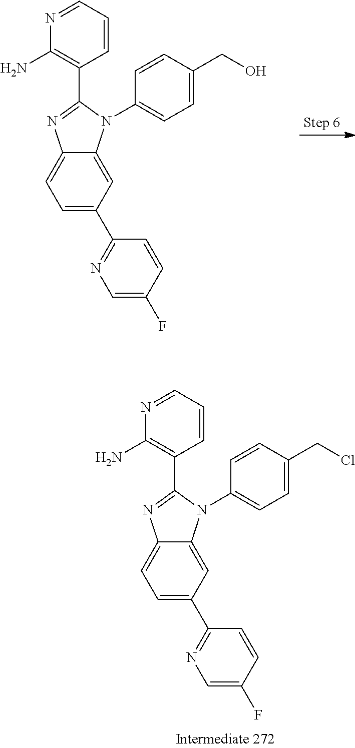 [SMILES: Nc1ncccc1-c1nc2ccc(-c3ccc(F)cn3)cc2n1-c1ccc(CO)cc1 ; Nc1ncccc1-c1nc2ccc(-c3ccc(F)cn3)cc2n1-c1ccc(CCl)cc1]

### Step 1: (4-((5-Bromo-2-nitrophenyl)amino)phenyl)methanol

To a solution of 4-bromo-2-fluoro-1-nitrobenzene (9 g, 40.91 mmol) in 1,4-dioxane (100 mL) were added DIEA (15.86 g, 122.73 mmol) and (4-aminophenyl)methanol (5.04 g, 40.91 mmol). The mixture was degassed and purged with N₂ three times and stirred at 80° C. for 16 hr under N₂. The reaction mixture was diluted with H₂O (500 mL) and extracted with CH₂Cl₂ (500 mL×3). The combined organic layers were washed with brine (200 mL), dried over Na₂SO₄, filtered and concentrated under reduced pressure. After purified by silica gel flash chromatography (SiO₂, petroleum ether/ethyl acetate=99/1 to 7/3), (4-((5-bromo-2-nitrophenyl)amino)phenyl)methanol (3.1 g, yield: 23%) was obtained as a yellow solid. ¹H NMR (400 MHz, Dimethylsulfoxide-d₆) δ 9.48 (s, 1H), 8.04 (d, J=9.2 Hz, 1H), 7.40 (d, J=8.4 Hz, 2H), 7.29 (d, J=8.0 Hz, 2H), 7.10 (s, 1H), 7.00 (dd, J=9.2, 7.2 Hz, 1H), 5.23-5.21 (m, 1H), 4.53-4.51 (m, 2H).

### Step 2: 4-((5-Bromo-2-nitrophenyl)amino)benzyl acetate

To a solution of (4-((5-bromo-2-nitrophenyl)amino)phenyl)methanol (2.54 g, 7.86 mmol) and TEA (2.39 g, 25.6 mmol) in CH₂Cl₂ (30 mL) were added Ac₂O (802 mg, 7.86 mmol) and DMAP (96.0 mg, 786 μmol). The mixture was stirred at 0° C. for 1 hr. The reaction mixture was diluted with H₂O (500 mL) and extracted with CH₂Cl₂ (500 mL×3). The combined organic layers were washed with brine (200 mL), dried over Na₂SO₄, filtered and concentrated under reduced pressure. After purified by silica gel flash chromatography (Eluent of 0˜15% EtOAc in petroleum ether), 4-((5-bromo-2-nitrophenyl)amino)benzyl acetate (2.7 g, yield: 92%) was obtained as a yellow solid. ¹H NMR (400 MHz, Chloroform-d) δ 9.41 (s, 1H), 7.98 (d, J=9.2 Hz, 1H), 7.35 (d, J=8.4 Hz, 2H), 7.17-7.15 (m, 3H), 6.80 (dd, J=9.2, 2.0 Hz, 1H), 5.04 (s, 2H), 2.05 (s, 3H).

### Step 3: 4-(2-(2-Aminopyridin-3-yl)-6-bromo-1H-benzo[d]imidazol-1-yl)benzyl acetate

To a solution of 4-((5-bromo-2-nitrophenyl)amino)benzyl acetate (2.7 g, 7.39 mmol) in DMSO (10 mL) were added Na₂S₂O₄ (5.12 g, 29.6 mmol, 87% purity) and 2-aminonicotinaldehyde (1.08 g, 8.87 mmol). The mixture was stirred at 100° C. for 3 hr. The reaction mixture was quenched with H₂O (500 mL) and extracted with EtOAc (500 mL×2). The combined organic layers were washed with brine (500 mL), dried over Na₂SO₄, filtered and concentrated under reduced pressure. After purified by silica gel flash chromatography (Eluent of 0%˜40% EtOAc in petroleum ether), 4-(2-(2-aminopyridin-3-yl)-6-bromo-1H-benzo[d]imidazol-1-yl)benzyl acetate (1.1 g, yield: 33%) was obtained as a light yellow solid. MS: m/z=437.0, 438.9 [M+H]+. ¹H NMR (400 MHz, Chloroform-d) δ 8.05 (dd, J=4.8, 1.6 Hz, 1H), 7.68 (d, J=8.4 Hz, 1H), 7.53 (d, J=8.4 Hz, 2H), 7.44 (dd, J=8.4, 1.6 Hz, 1H), 7.34 (d, J=1.6 Hz, 1H), 7.31 (d, J=8.4 Hz, 2H), 7.02 (dd, J=7.6, 1.6 Hz, 1H), 6.59 (s, 2H), 6.35 (dd, J=4.8, 7.8 Hz, 1H), 5.21 (s, 2H), 2.18 (s, 3H).

### Step 4: 4-(2-(2-Aminopyridin-3-yl)-6-(5-fluoropyridin-2-yl)-1H-benzo[d]imidazol-1-yl)benzyl acetate

A mixture of 4-(2-(2-aminopyridin-3-yl)-6-bromo-1H-benzo[d]imidazol-1-yl)benzyl acetate (500 mg, 1.14 mmol), (5-fluoropyridin-2-yl)boronic acid (483 mg, 3.43 mmol), Pd(dppf)Cl₂ (83.7 mg, 114 μmol), Cs₂CO₃ (1.12 g, 3.43 mmol) and CuBr 65.6 mg, 457 μmol) in 1,4-dioxane (7.5 mL) and H₂O (1.5 mL) was degassed and purged with N₂ three times. The mixture was stirred at 100° C. for 16 hr under N₂ atmosphere. The reaction mixture was quenched with H₂O (100 mL), diluted with CH₂Cl₂ (100 mL), and extracted with CH₂Cl₂ (100 mL×2). The combined organic layers were washed with brine (100 mL), dried over Na₂SO₄, filtered and concentrated under reduced pressure. After purified by silica gel flash chromatography (Eluent of 0%˜56% EtOAc in petroleum ether), 4-(2-(2-aminopyridin-3-yl)-6-(5-fluoropyridin-2-yl)-1H-benzo[d]imidazol-1-yl)benzyl acetate (380 mg, yield: 59%) was obtained as a light yellow solid. MS: m/z=454.2 [M+H]+.

### Step 5: (4-(2-(2-Aminopyridin-3-yl)-6-(5-fluoropyridin-2-yl)-1H-benzo[d]imidazol-1-yl)phenyl)methanol

To a solution of 4-(2-(2-aminopyridin-3-yl)-6-(5-fluoropyridin-2-yl)-1H-benzo[d]imidazol-1-yl)benzyl acetate (380 mg, 838 μmol) in THF (4 mL) and MeOH (4 mL) was added K₂CO₃ (347 mg, 2.51 mmol) in H₂O (4 mL). The mixture was stirred at 25° C. for 1 hr. The reaction mixture was quenched with H₂O (100 mL) at 25° C. and extracted with EtOAc (100 mL×2). The combined organic layers were washed with brine (100 mL×2), dried over Na₂SO₄, filtered and concentrated under reduced pressure. (4-(2-(2-Aminopyridin-3-yl)-6-(5-fluoropyridin-2-yl)-1H-benzo[d]imidazol-1-yl)phenyl)methanol (340 mg, yield: 85%) was obtained as a light yellow solid. MS: m/z=412.2 [M+H]⁺.

### Step 6: 3-(1-(4-(Chloromethyl)phenyl)-6-(5-fluoropyridin-2-yl)-1H-benzo[d]imidazol-2-yl)pyridin-2-amine

To a solution of (4-(2-(2-aminopyridin-3-yl)-6-(5-fluoropyridin-2-yl)-1H-benzo[d]imidazol-1-yl)phenyl)methanol (400 mg, 972 μmol) in CH₂Cl₂ (6 mL) was added SOCl₂ (347 mg, 2.92 mmol). The mixture was stirred at 40° C. for 0.5 hr. The reaction was concentrated under reduced pressure to give 3-(1-(4-(chloromethyl)phenyl)-6-(5-fluoropyridin-2-yl)-1H-benzo[d]imidazol-2-yl)pyridin-2-amine (Intermediate 272, 400 mg, HCl salt) as a light yellow solid. MS: m/z=430.1 [M+H]⁺.

### Intermediate 273: 3-(3-(4-(Chloromethyl)phenyl)-5-(5-fluoropyrimidin-2-yl)-3H-imidazo[4,5-b]pyridin-2-yl)pyridin-2-amine

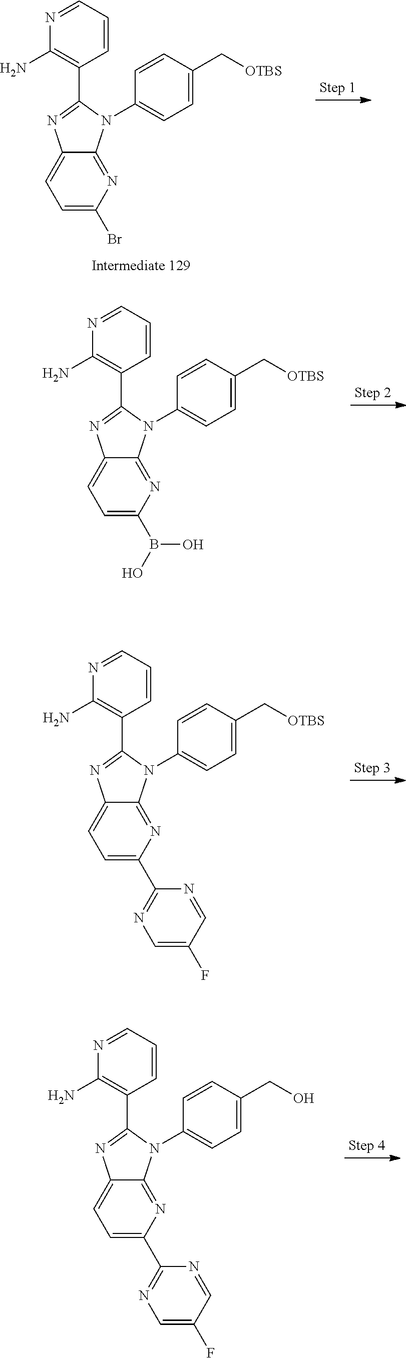 [SMILES: CC(C)(C)[Si](C)(C)OCc1ccc(-n2c(-c3cccnc3N)nc3ccc(Br)nc32)cc1 ; CC(C)(C)[Si](C)(C)OCc1ccc(-n2c(-c3cccnc3N)nc3ccc(B(O)O)nc32)cc1 ; CC(C)(C)[Si](C)(C)OCc1ccc(-n2c(-c3cccnc3N)nc3ccc(-c4ncc(F)cn4)nc32)cc1 ; Nc1ncccc1-c1nc2ccc(-c3ncc(F)cn3)nc2n1-c1ccc(CO)cc1]

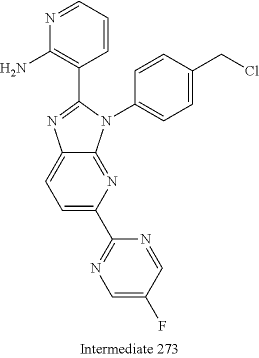 [SMILES: Nc1ncccc1-c1nc2ccc(-c3ncc(F)cn3)nc2n1-c1ccc(CCl)cc1]

### Step 1: (2-(2-Aminopyridin-3-yl)-3-(4-(((tert-butyldimethylsilyl)oxy)methyl)phenyl)-3H-imidazo[4,5-b]pyridin-5-yl)boronic acid

A mixture of Intermediate 129 (500 mg, 947 μmol), 4,4,5,5-tetramethyl-2-(4,4,5,5-tetramethyl-1,3,2-dioxaborolan-2-yl)-1,3,2-dioxaborolane (497 mg, 1.96 mmol), KOAc (288 mg, 2.94 mmol), and Pd(dppf)Cl₂ (72 mg, 98 μmol) in 1,4-dioxane (5 mL) was degassed and purged with N₂ three times. The mixture was stirred at 80° C. for 16 hr under N₂ atmosphere. (2-(2-Aminopyridin-3-yl)-3-(4-(((tert-butyldimethylsilyl)oxy)methyl)phenyl)-3H-imidazo[4,5-b]pyridin-5-yl)boronic acid was obtained as a black brown liquid, which was used in the next step without work-up and purification. MS: m/z=476.3 [M+H]⁺.

### Step 2: 3-(3-(4-(((tert-Butyldimethylsilyl)oxy)methyl)phenyl)-5-(5-fluoropyrimidin-2-yl)-3H-imidazo[4,5-b]pyridin-2-yl)pyridin-2-amine

A mixture of (2-(2-aminopyridin-3-yl)-3-(4-(((tert-butyldimethylsilyl)oxy)methyl)phenyl)-3H-imidazo[4,5-b]pyridin-5-yl)boronic acid (500 mg, 1.05 mmol), 2-chloro-5-fluoropyrimidine (139 mg, 1.05 mmol), Pd(dppf)Cl₂ (76.9 mg, 105 μmol), and Cs₂CO₃ (1.03 g, 3.16 mmol) in 1,4-dioxane (4 mL) and H₂O (1 mL) was degassed and purged with N₂ three times. The mixture was stirred at 90° C. for 16 hr under N₂ atmosphere. The reaction mixture was diluted with H₂O (20 mL) and extracted with EtOAc (20 mL). The combined organic layers were washed with brine (30 mL), dried over Na₂SO₄, filtered and concentrated under reduced pressure. After purified by silica gel flash chromatography (Eluent of 0˜50% EtOAc in petroleum ether), 3-(3-(4-(((tert-butyldimethylsilyl)oxy)methyl)phenyl)-5-(5-fluoropyrimidin-2-yl)-3H-imidazo[4,5-b]pyridin-2-yl)pyridin-2-amine (410 mg, yield: 65% for two steps) was obtained as a black brown solid. MS: m/z=528.2 [M+H]⁺. ¹H NMR (400 MHz, Dimethysulfoxide-d₆) δ 9.00 (s, 2H), 8.43 (d, J=8.4 Hz, 1H), 8.34 (d, J=8.4 Hz, 1H), 8.08-7.91 (m, 1H), 7.53-7.44 (m, 4H), 7.19 (dd, J=7.6, 1.6 Hz, 1H), 7.02 (s, 2H), 6.38 (dd, J=7.6, 4.8 Hz, 1H), 4.82 (s, 2H), 0.93 (s, 9H), 0.12 (s, 6H).

### Step 3: (4-(2-(2-Aminopyridin-3-yl)-5-(5-fluoropyrimidin-2-yl)-3H-imidazo[4,5-b]pyridin-3-yl)phenyl)methanol

To a solution of 3-(3-(4-(((tert-butyldimethylsilyl)oxy)methyl)phenyl)-5-(5-fluoropyrimidin-2-yl)-3H-imidazo[4,5-b]pyridin-2-yl)pyridin-2-amine (410 mg, 777 μmol) in THF (5 mL) was added TBAF (406 mg, 1.55 mmol, 1 M in THF). The mixture was stirred at 25° C. for 1 hr. The mixture was diluted with H₂O (20 mL) and extracted with EtOAc (20 mL×2). The combined organic layers were washed with brine (20 mL), dried over Na₂SO₄, filtered and concentrated under reduced pressure. After triturated with EtOAc (10 mL) at 25° C. for 1 hr, (4-(2-(2-aminopyridin-3-yl)-5-(5-fluoropyrimidin-2-yl)-3H-imidazo[4,5-b]pyridin-3-yl)phenyl)methanol (260 mg, crude) was obtained as a black brown solid. MS: m/z=414.1 [M+H]⁺.

### Step 4: 3-(3-(4-(Chloromethyl)phenyl)-5-(5-fluoropyrimidin-2-yl)-3H-imidazo[4,5-b]pyridin-2-yl)pyridin-2-amine

To a solution of (4-(2-(2-aminopyridin-3-yl)-5-(5-fluoropyrimidin-2-yl)-3H-imidazo[4,5-b]pyridin-3-yl)phenyl)methanol (260 mg, 629 μmol) in CH₂Cl₂ (4 mL) was added SOCl₂ (2 mL). The mixture was stirred at 25° C. for 1 hr. The mixture was concentrated under reduced pressure to give 3-(3-(4-(chloromethyl)phenyl)-5-(5-fluoropyrimidin-2-yl)-3H-imidazo[4,5-b]pyridin-2-yl)pyridin-2-amine (Intermediate 273, 300 mg, crude, HCl salt) as a black brown solid. The crude product was used into the next step without further purification. MS: m/z=432.1 [M+H]⁺.

### Intermediate 274: 3-(3-(4-(Chloromethyl)phenyl)-5-(5-methylpyrimidin-2-yl)-3H-imidazo[4,5-b]pyridin-2-yl)pyridin-2-amine

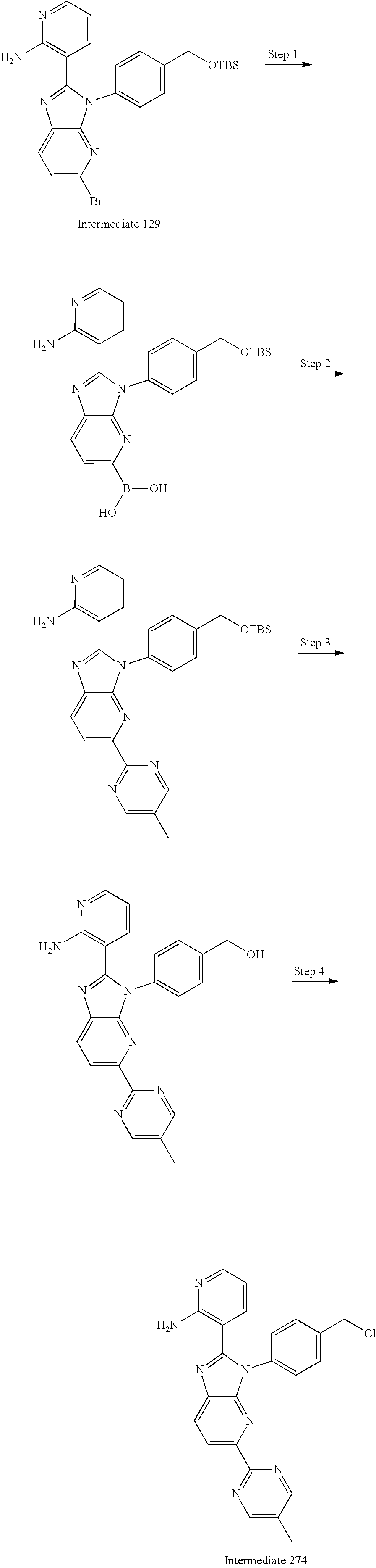 [SMILES: CC(C)(C)[Si](C)(C)OCc1ccc(-n2c(-c3cccnc3N)nc3ccc(Br)nc32)cc1 ; CC(C)(C)[Si](C)(C)OCc1ccc(-n2c(-c3cccnc3N)nc3ccc(B(O)O)nc32)cc1 ; Cc1cnc(-c2ccc3nc(-c4cccnc4N)n(-c4ccc(CO[Si](C)(C)C(C)(C)C)cc4)c3n2)nc1 ; Cc1cnc(-c2ccc3nc(-c4cccnc4N)n(-c4ccc(CO)cc4)c3n2)nc1 ; Cc1cnc(-c2ccc3nc(-c4cccnc4N)n(-c4ccc(CCl)cc4)c3n2)nc1]

### Step 1: (2-(2-Aminopyridin-3-yl)-3-(4-(((tert-butyldimethylsilyl)oxy)methyl)phenyl)-3H-imidazo[4,5-b]pyridin-5-yl)boronic acid

A mixture of Intermediate 129 (500 mg, 947 μmol), 4,4,4′,4′,5,5,5′,5′-octamethyl-2,2′-bi(1,3,2-dioxaborolane) (497 mg, 1.96 mmol), KOAc (288 mg, 2.94 mmol), Pd(dppf)Cl₂ (72 mg, 98 t mol) in 1,4-dioxane (5 mL) was degassed and purged with N₂ three times. The mixture was stirred at 80° C. for 16 hr under N₂ atmosphere. (2-(2-Aminopyridin-3-yl)-3-(4-(((tert-butyldimethylsilyl)oxy)methyl)phenyl)-3H-imidazo[4,5-b]pyridin-5-yl)boronic acid was obtained as a black liquid, which was used in the next step without work-up and purification. MS: m/z=476.2 [M+H]⁺.

### Step 2: 3-(3-(4-(((tert-Butyldimethylsilyl)oxy)methyl)phenyl)-5-(5-methylpyrimidin-2-yl)-3H-imidazo[4,5-b]pyridin-2-yl)pyridin-2-amine

A mixture of (2-(2-aminopyridin-3-yl)-3-(4-(((tert-butyldimethylsilyl)oxy)methyl)phenyl)-3H-imidazo[4,5-b]pyridin-5-yl)boronic acid (400 mg, 841 μmol), 2-chloro-5-methylpyrimidine (108 mg, 841 μmol), Pd(dppf)Cl₂ (61.56 mg, 84 μmol), Cs₂CO₃ (822 mg, 2.52 mmol) and H₂O (0.5 mL) in 1,4-dioxane (2 mL) was degassed and purged with N₂ three times. The mixture was stirred at 90° C. for 16 hr under N₂ atmosphere. The reaction mixture was diluted with H₂O (10 mL) and extracted with EtOAc (10 mL×3). The combined organic layers were washed with brine (20 mL), dried over Na₂SO₄, filtered and concentrated under reduced pressure. After purified by silica gel flash chromatography (Eluent of 0%˜50% EtOAc in petroleum ether), 3-(3-(4-(((tert-butyldimethylsilyl)oxy)methyl)phenyl)-5-(5-methylpyrimidin-2-yl)-3H-imidazo[4,5-b]pyridin-2-yl)pyridin-2-amine (240 mg, yield: 44% for two steps) was obtained as a black brown solid. MS: m/z=524.3 [M+H]⁺.1H NMR (400 MHz, Dimethysulfoxide-d₆) δ 8.89-8.66 (m, 2H), 8.45 (d, J=8.0 Hz, 1H), 8.31 (d, J=8.0 Hz, 1H), 8.05-7.90 (m, 1H), 7.54-7.42 (m, 4H), 7.19 (d, J=7.2 Hz, 1H), 7.01 (br s, 2H), 6.44-6.30 (m, 1H), 4.83 (s, 2H), 2.32 (s, 3H), 0.94 (s, 9H), 0.12 (s, 6H)

### Step 3: (4-(2-(2-Aminopyridin-3-yl)-5-(5-methylpyrimidin-2-yl)-3H-imidazo[4,5-b]pyridin-3-yl)phenyl)methanol

To a solution of 3-(3-(4-(((tert-butyldimethylsilyl)oxy)methyl)phenyl)-5-(5-methylpyrimidin-2-yl)-3H-imidazo[4,5-b]pyridin-2-yl)pyridin-2-amine (220 mg, 420 μmol) in THF (2 mL) was added TBAF (220 mg, 1 M in THF). The mixture was stirred at 25° C. for 1 hr. The reaction mixture was diluted with H₂O (30 mL) and extracted with EtOAc (20 mL). The combined organic layers were washed with brine (30 mL), dried over Na₂SO₄, filtered and concentrated under reduced pressure. After triturated with EtOAc in petroleum ether=1:1 (10 mL) at 25° C. for 16 hr, (4-(2-(2-aminopyridin-3-yl)-5-(5-methylpyrimidin-2-yl)-3H-imidazo[4,5-b]pyridin-3-yl)phenyl)methanol (180 mg) was obtained as a yellow solid. MS: m/z=410.2 [M+H]⁺.

### Step 4: 3-(3-(4-(Chloromethyl)phenyl)-5-(5-methylpyrimidin-2-yl)-3H-imidazo[4,5-b]pyridin-2-yl)pyridin-2-amine

To a solution of (4-(2-(2-aminopyridin-3-yl)-5-(5-methylpyrimidin-2-yl)-3H-imidazo[4,5-b]pyridin-3-yl)phenyl)methanol in CH₂C12 (2 mL) was added SOCl₂ (1 mL). The mixture was stirred at 25° C. for 0.5 hr. The mixture was concentrated under reduced pressure to give 3-(3-(4-(chloromethyl)phenyl)-5-(5-methylpyrimidin-2-yl)-3H-imidazo[4,5-b]pyridin-2-yl)pyridin-2-amine (Intermediate 274, 142 mg, HCl salt) as a light yellow solid. MS: m/z=428.2 [M+H]r.

### Intermediate 275: 3-(3-(4-(Chloromethyl)phenyl)-5-(5-fluoropyrazin-2-yl)-3H-imidazo[4,5-b]pyridin-2-yl)pyridin-2-amine

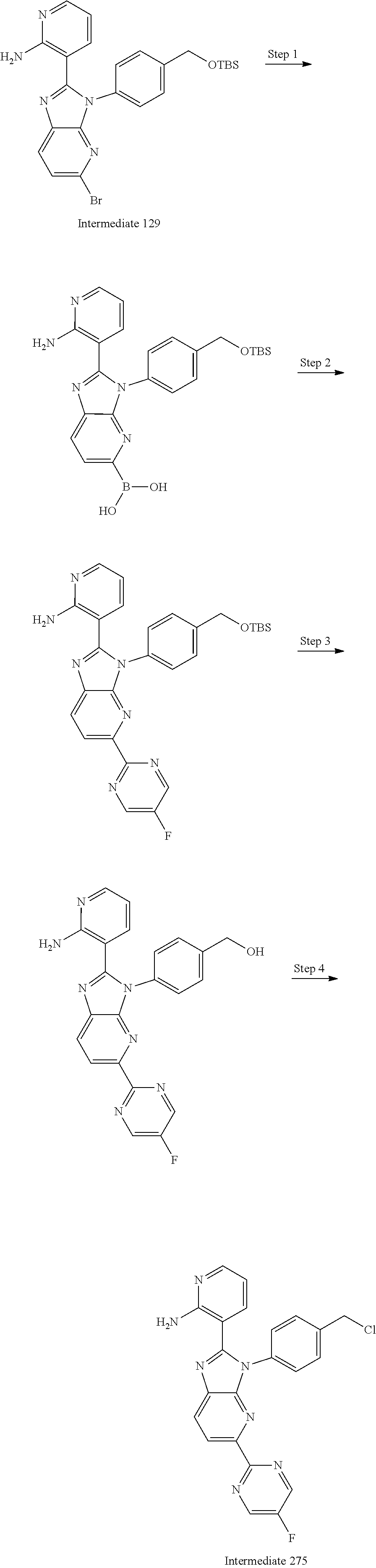 [SMILES: CC(C)(C)[Si](C)(C)OCc1ccc(-n2c(-c3cccnc3N)nc3ccc(Br)nc32)cc1 ; CC(C)(C)[Si](C)(C)OCc1ccc(-n2c(-c3cccnc3N)nc3ccc(B(O)O)nc32)cc1 ; CC(C)(C)[Si](C)(C)OCc1ccc(-n2c(-c3cccnc3N)nc3ccc(-c4ncc(F)cn4)nc32)cc1 ; Nc1ncccc1-c1nc2ccc(-c3ncc(F)cn3)nc2n1-c1ccc(CO)cc1 ; Nc1ncccc1-c1nc2ccc(-c3ncc(F)cn3)nc2n1-c1ccc(CCl)cc1]

### Step 1: (2-(2-Aminopyridin-3-yl)-3-(4-(((tert-butyldimethylsilyl)oxy)methyl)phenyl)-3H-imidazo[4,5-b]pyridin-5-yl)boronic acid

A mixture of Intermediate 129 (1 g, 1.96 mmol), 4,4,4′,4′,5,5,5′,5′-octamethyl-2,2′-bi(1,3,2-dioxaborolane) (995 mg, 3.92 mmol), KOAc (577 mg, 5.88 mmol) and Pd(dppf)Cl₂ (143 mg, 196 mmol) in 1,4-dioxane (5 mL) was degassed and purged with N₂ three times. The mixture was stirred at 90° C. for 16 hr under N₂ atmosphere. (2-(2-aminopyridin-3-yl)-3-(4-(((tert-butyldimethylsilyl)oxy)methyl)phenyl)-3H-imidazo[4,5-b]pyridin-5-yl)boronic acid was obtained as a black liquid, which was used in the next step without work-up and purification. MS: m/z=476.1 [M+H]⁺.

### Step 2: 3-(3-(4-(((tert-Butyldimethylsilyl)oxy)methyl)phenyl)-5-(5-fluoropyrazin-2-yl)-3H-imidazo[4,5-b]pyridin-2-yl)pyridin-2-amine

A mixture of (2-(2-aminopyridin-3-yl)-3-(4-(((tert-butyldimethylsilyl)oxy)methyl)phenyl)-3H-imidazo[4,5-b]pyridin-5-yl)boronic acid (410 mg, 862 μmol), 2-bromo-5-fluoropyrazine (168 mg, 949 μmol), Pd(dppf)Cl₂ (63.1 mg, 86.2 μmol) and Cs₂CO₃ (843 mg, 2.59 mmol) in 1,4-dioxane (4 mL) and H₂O (I mL) was degassed and purged with N₂ three times. The mixture was stirred at 90° C. for 16 hr under N₂ atmosphere. The reaction mixture was poured into H₂O (15 mL) and extracted with EtOAc (15 mL×3). The combined organic layers were dried over anhydrous Na₂SO₄, filtered and concentrated under reduced pressure. After purified by silica gel flash chromatography (Eluent of 0%˜25% EtOAc in petroleum ether), 3-(3-(4-(((tert-butyldimethylsilyl)oxy)methyl)phenyl)-5-(5-fluoropyrazin-2-yl)-3H-imidazo[4,5-b]pyridin-2-yl)pyridin-2-amine (480 mg, yield: 40% for two steps) was obtained as a brown solid. MS: m/z=528.3 [M+H]⁺.

### Step 3: 4-(2-(2-Aminopyridin-3-yl)-5-(5-fluoropyrazin-2-yl)-3H-imidazo[4,5-b]pyridin-3-yl)phenyl)methanol

To a solution of 3-(3-(4-(((tert-butyldimethylsilyl)oxy)methyl)phenyl)-5-(5-fluoropyrazin-2-yl)-3H-imidazo[4,5-b]pyridin-2-yl)pyridin-2-amine (480 mg, 910 μmol) in THF (4 mL) was added TBAF (1.5 mL, 1 M in THF). The mixture was stirred at 25° C. for 0.5 hr. The reaction mixture was poured into H₂O (15 mL) and extracted with EtOAc (15 mL×3). After purified by silica gel flash chromatography (Eluent of 0%˜65% EtOAc in petroleum ether), 4-(2-(2-aminopyridin-3-yl)-5-(5-fluoropyrazin-2-yl)-3H-imidazo[4,5-b]pyridin-3-yl)phenyl)methanol (153 mg, yield: 41%) was obtained as a yellow solid. MS: m/z=414.1 [M+H]⁺. ¹H NMR (400 MHz, Dimethysulfoxide-d₆) δ 9.03-8.88 (m, 1H), 8.72 (d, J=8.8 Hz, 1H), 8.42-8.25 (m, 2H), 8.01 (dd, J=4.8, 1.6 Hz, 1H), 7.55-7.39 (m, 4H), 7.34-7.16 (m, 1H), 7.08-6.89 (s, 2H), 6.44 (dd, J=8.0, 5.2 Hz, 1H), 5.49-5.22 (m, 1H), 4.61-4.60 (m, 2H). ¹⁹FNMR (400 MHz, Dimethysulfoxide-d₆) δ −82.690.

### Step 4: 3-(3-(4-(Chloromethyl)phenyl)-5-(5-fluoropyrazin-2-yl)-3H-imidazo[4,5-b]pyridin-2-yl)pyridin-2-amine

To a solution of 4-(2-(2-aminopyridin-3-yl)-5-(5-fluoropyrazin-2-yl)-3H-imidazo[4,5-b]pyridin-3-yl)phenyl)methanol (153 mg, 370 μmol) in CH₂Cl₂ (2 mL) was added SOCl₂ (1 mL). The mixture was stirred at 40° C. for 0.3 hr. The reaction was concentrated under reduced pressure to give 3-(3-(4-(chloromethyl)phenyl)-5-(5-fluoropyrazin-2-yl)-3H-imidazo[4,5-b]pyridin-2-yl)pyridin-2-amine (Intermediate 275, 146 mg, HCl salt) was obtained as a yellow solid. MS: m/z=432.1 [M+H]⁺.

### Intermediate 276: 5-Bromo-2-(fluoromethoxy)pyridine

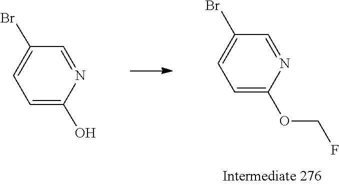 [SMILES: Oc1ccc(Br)cn1 ; FCOc1ccc(Br)cn1]

To a solution of 5-bromopyridin-2-ol (1 g, 5.8 mmol) and K₂CO₃ (2.4 g, 17.2 mmol) in DMF (10 mL) was added fluoromethyl 4-methylbenzenesulfonate (1.2 g, 5.8 mmol) at 25° C. The mixture was stirred at 50° C. for 2 hr. The mixture was quenched with H₂O (50 mL) and extracted with CH₂Cl₂ (20 mL×2). The combined organic layers were washed with brine (50 mL×3), dried over Na₂SO₄, filtered and concentrated. After purified by silica gel flash chromatography (Eluent of 0˜15% Ethyl acetate in petroleum ether), 5-bromo-2-(fluoromethoxy)pyridine (Intermediate 276, 1 g, yield: 84%) was obtained as a yellow oil. MS: m/z=205.8, 207.8 [M+H]⁺. ¹H NMR (400 MHz, Dimethylsulfoxide-d₆) δ 8.18 (d, J=2.8 Hz, 1H), 7.61 (dd, J=9.6, 2.8 Hz, 1H), 6.47 (d, J=9.6 Hz, 1H), 5.90-5.75 (m, 2H). ¹⁹F NMR (400 MHz, Dimethylsulfoxide-d₆) δ −172.73.

### Intermediate 277: 3-(3-(4-(Chloromethyl)phenyl)-5-(6-(fluoromethoxy)pyridin-3-yl)-3H-imidazo[4,5-b]pyridin-2-yl)pyridin-2-amine

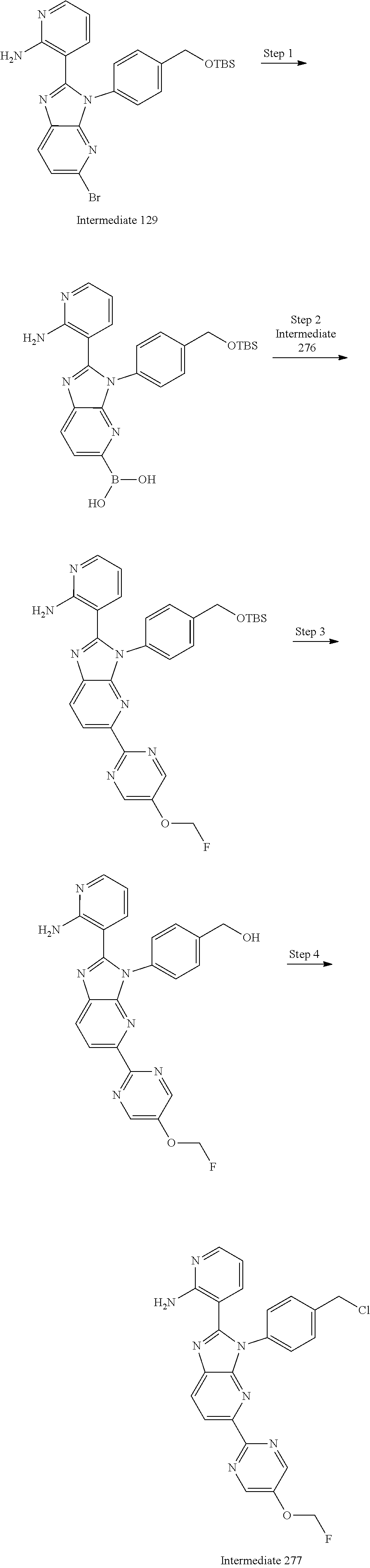 [SMILES: CC(C)(C)[Si](C)(C)OCc1ccc(-n2c(-c3cccnc3N)nc3ccc(Br)nc32)cc1 ; CC(C)(C)[Si](C)(C)OCc1ccc(-n2c(-c3cccnc3N)nc3ccc(B(O)O)nc32)cc1 ; CC(C)(C)[Si](C)(C)OCc1ccc(-n2c(-c3cccnc3N)nc3ccc(-c4ncc(OCF)cn4)nc32)cc1 ; Nc1ncccc1-c1nc2ccc(-c3ncc(OCF)cn3)nc2n1-c1ccc(CO)cc1 ; Nc1ncccc1-c1nc2ccc(-c3ncc(OCF)cn3)nc2n1-c1ccc(CCl)cc1]

### Step 1: (2-(2-Aminopyridin-3-yl)-3-(4-(((tert-butyldimethylsilyl)oxy)methyl)phenyl)-3H-imidazo[4,5-b]pyridin-5-yl)boronic acid

A mixture of Intermediate 129 (1 g, 1.96 mmol), 4,4,4′,4′,5,5,5′,5′-octamethyl-2,2′-bi(1,3,2-dioxaborolane) (995 mg, 3.92 mmol), KOAc (577 mg, 5.88 mmol) and Pd(dppf)Cl₂ (143 mg, 196 μmol) in 1,4-dioxane (10 mL) was degassed and purged with N₂ three times. The mixture was stirred at 90° C. for 8 hr under N₂ atmosphere. (2-(2-aminopyridin-3-yl)-3-(4-(((tert-butyldimethylsilyl)oxy)methyl)phenyl)-3H-imidazo[4,5-b]pyridin-5-yl)boronic acid was obtained as a black liquid, which was used in the next step without work-up and purification. MS: m/z=476.2 [M+H]⁺.

### Step 2: 3-(3-(4-(((tert-Butyldimethylsilyl)oxy)methyl)phenyl)-5-(6-(fluoromethoxy)pyridin-3-yl)-3H-imidazo[4,5-b]pyridin-2-yl)pyridin-2-amine

A mixture of (2-(2-aminopyridin-3-yl)-3-(4-(((tert-butyldimethylsilyl)oxy)methyl)phenyl)-3H-imidazo[4,5-b]pyridin-5-yl)boronic acid (466 mg, 980 μmol), Intermediate 276 (202 mg, 980 μmol), Pd(dppf)Cl₂ (71.7 mg, 98 μmol) and Cs₂CO₃ (958 mg, 2.94 mmol) in 1,4-dioxane (5 mL) and H₂O (1 mL) was degassed and purged with N₂ for 3 times. The mixture was stirred at 90° C. for 16 hr under N₂ atmosphere. The reaction mixture was quenched with H₂O (5 mL) and extracted with EtOAc (10 mL×3). The combined organic layers were washed with brine (30 mL), dried over Na₂SO₄, filtered and concentrated under reduced pressure. After purified by silica gel flash chromatography (Eluent of 15%˜37% EtOAc in petroleum ether), 3-(3-(4-(((tert-butyldimethylsilyl)oxy)methyl)phenyl)-5-(6-(fluoromethoxy)pyridin-3-yl)-3H-imidazo[4,5-b]pyridin-2-yl)pyridin-2-amine (300 mg, yield: 55% for two steps) was obtained as a purple solid. MS: m/z=557.7 [M+H]⁺. ¹H NMR (400 MHz, Dimethylsulfoxide-d₆) δ 8.90 (d, J=2.0 Hz, 1H), 8.41 (dd, J=8.8, 2.4 Hz, 1H), 8.29 (d, J=8.4 Hz, 1H), 8.05-7.97 (m, 2H), 7.51-7.46 (m, 4H), 7.20 (dd, J=7.6, 2.0 Hz, 1H), 7.10-7.00 (m, 3H), 6.39 (dd, J=7.6, 4.8 Hz, 1H), 6.21-6.04 (m, 2H), 4.82 (s, 2H), 0.92 (s, 9H), 0.11 (s, 6H). ¹⁹F NMR (400 MHz, Dimethylsulfoxide-d₆) δ −154.37.

### Step 3: (4-(2-(2-Aminopyridin-3-yl)-5-(6-(fluoromethoxy)pyridin-3-yl)-3H-imidazo[4,5-b]pyridin-3-yl)phenyl)methanol

To a solution of 3-(3-(4-(((tert-butyldimethylsilyl)oxy)methyl)phenyl)-5-(6-(fluoromethoxy)pyridin-3-yl)-3H-imidazo[4,5-b]pyridin-2-yl)pyridin-2-amine (300 mg, 539 μmol) in THF (5 mL) was added TBAF (423 mg, 1.62 mmol, 1 M in THF). The mixture was stirred at 25° C. for 1 hr. The reaction mixture was quenched with H₂O (5 mL) at 25° C. and extracted with EtOAc (10 mL×3). The combined organic layers were washed with brine (50 mL×3), dried over Na₂SO₄, filtered and concentrated under reduced pressure. (4-(2-(2-Aminopyrdin-3-yl)-5-(6-(fluoromethoxy)pyridin-3-yl)-3H-imidazo[4,5-b]pyridin-3-yl)phenyl)methanol (240 mg) was obtained as a brown solid. MS: m/z=443.1 [M+H]⁺. ¹H NMR (400 MHz, Dimethylsulfoxide-d₆) δ 8.94-8.87 (m, 1H), 8.46-8.38 (m, 1H), 8.29 (d, J=8.0 Hz, 1H), 8.08-7.93 (m, 2H), 7.56-7.36 (m, 4H), 7.23 (d, J=7.6 Hz, 1H), 7.09 (d, J=8.4 Hz, 1H), 7.05-6.92 (m, 2H), 6.47-6.37 (m, 1H), 6.25-5.99 (m, 2H), 5.48-5.29 (m, 1H), 4.70-4.49 (m, 2H). ¹⁹F NMR (400 MHz, Dimethylsulfoxide-d₆) δ −154.36.

### Step 4: 3-(3-(4-(Chloromethyl)phenyl)-5-(6-(fluoromethoxy)pyridin-3-yl)-3H-imidazo[4,5-b]pyridin-2-yl)pyridin-2-amine

To a solution of (4-(2-(2-aminopyridin-3-yl)-5-(6-(fluoromethoxy)pyridin-3-yl)-3H-imidazo[4,5-b]pyridin-3-yl)phenyl)methanol (240 mg, 542 μmol) in CH₂Cl₂ (5 mL) was added SOCl₂ (194 mg, 1.63 mmol). The mixture was stirred at 40° C. for 0.5 hr. The reaction was concentrated under reduced pressure to give 3-(3-(4-(chloromethyl)phenyl)-5-(6-(fluoromethoxy)pyridin-3-yl)-3H-imidazo[4,5-b]pyridin-2-yl)pyridin-2-amine (Intermediate 277, 250 mg, HCl salt) as a yellow solid. MS: m/z=461.0 [M+H]⁺.

### Intermediate 278: 5-Bromo-1-(fluoromethyl)pyridin-2(1H)-one

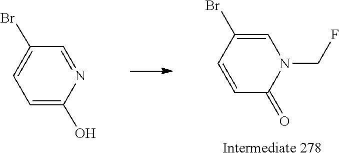 [SMILES: Oc1ccc(Br)cn1 ; O=c1ccc(Br)cn1CF]

To a solution of 5-bromopyridin-2-ol (3 g, 17.2 mmol), and K₂CO₃ (7.1 g, 52 mmol) in DMF (10 mL) was added bromofluoromethane (2.9 g, 26 mmol) at 0° C. The mixture was stirred at 0° C. for 2 hr. The mixture was quenched with H₂O (50 mL) and extracted with CH₂Cl₂ (20 mL×2). The combined organic layers were washed with brine (50 mL×3), dried over Na₂SO₄, filtered and concentrated. After purified by silica gel flash chromatography (Eluent of 0˜15% Ethyl acetate in petroleum ether), 5-Bromo-1-(fluoromethyl)pyridin-2(1H)-one (Intermediate 278, 2.7 g, yield: 76%) was obtained as a yellow oil. MS: m/z=205.7, 207.7 [M+H]⁺. ¹H NMR (400 MHz, Dimethylsulfoxide-d₆) δ 8.19 (d, J=2.8 Hz, 1H), 7.61 (dd, J=9.6, 2.8 Hz, 1H), 6.47 (d, J=9.6 Hz, 1H), 5.88 (d, J=51.2 Hz, 2H). ¹⁹F NMR (400 MHz, Dimethylsulfoxide-d₆) δ −172.733.

### Intermediate 279: 5-(2-(2-Aminopyridin-3-yl)-3-(4-(chloromethyl)phenyl)-3H-imidazo[4,5-b]pyridin-5-yl)-1-(fluoromethyl)pyridin-2(1H)-one

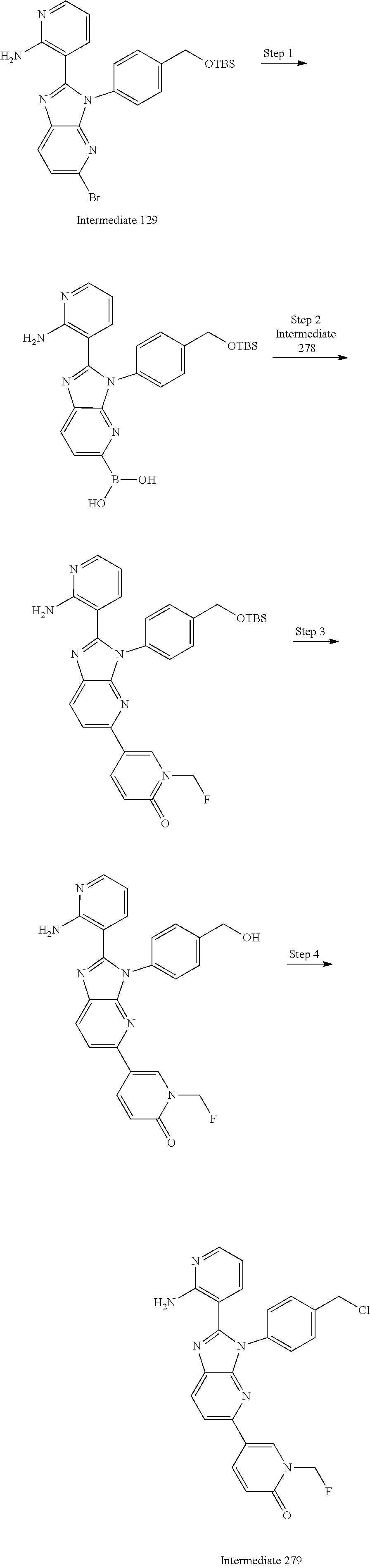

### Step 1: (2-(2-Aminopyridin-3-yl)-3-(4-(((tert-butyldimethylsilyl)oxy)methyl)phenyl)-3H-imidazo[4,5-b]pyridin-5-yl)boronic acid

A mixture of Intermediate 129 (1.5 g, 2.9 mmol), 4,4,4′,4′,5,5,5′,5′-octamethyl-2,2′-bi(1,3,2-dioxaborolane) (1.1 g, 4.4 mmol), KOAc (865 mg, 8.8 mmol) and Pd(dppf)Cl₂ (120 mg, 147 μmol) in 1,4-dioxane (15 mL) was degassed and purged with N₂ for 3 times. The mixture was stirred at 110° C. for 2 hr under N₂ atmosphere. (2-(2-Aminopyridin-3-yl)-3-(4-(((tert-butyldimethylsilyl)oxy)methyl)phenyl)-3H-imidazo[4,5-b]pyridin-5-yl)boronic acid was obtained as a black liquid, which was used in the next step without work-up and purification. MS: m/z=476.2 [M+H]⁺.

### Step 2: 5-(2-(2-Aminopyridin-3-yl)-3-(4-(((tert-butyldimethylsilyl)oxy)methyl)phenyl)-3H-imidazo[4,5-b]pyridin-5-yl)-1-(fluoromethyl)pyridin-2(1H)-one

A mixture of (2-(2-aminopyridin-3-yl)-3-(4-(((tert-butyldimethylsilyl)oxy)methyl)phenyl)-3H-imidazo[4,5-b]pyridin-5-yl)boronic acid (931 mg, 1.96 mmol), Intermediate 278 (403 mg, 1.96 mmol), Pd(dppf)Cl₂ (143 mg, 196 μmol) and Cs₂CO₃ (1.9 g, 5.87 mmol) in 1,4-dioxane (10 mL) and H₂O (2 mL) was degassed and purged with N₂ three times and stirred at 90° C. for 12 hr under N₂ atmosphere. The reaction mixture was quenched with H₂O (30 mL) and extracted with EtOAc (20 mL×3). The combined organic layers were washed with brine (60 mL), dried over Na₂SO₄, filtered and concentrated under reduced pressure. After purified by silica gel flash chromatography (Eluent of 20%˜65% EtOAc in petroleum ether), 5-(2-(2-aminopyridin-3-yl)-3-(4-(((tert-butyldimethylsilyl)oxy)methyl)phenyl)-3H-imidazo[4,5-b]pyridin-5-yl)-1-(fluoromethyl)pyridin-2(1H)-one (570 mg, yield: 52% for two steps) was obtained as a brown solid. MS: m/z=557.2 [M+H]⁺.

### Step 3: 5-(2-(2-Aminopyridin-3-yl)-3-(4-(hydroxymethyl)phenyl)-3H-imidazo[4,5-b]pyridin-5-yl)-1-(fluoromethyl)pyridin-2(1H)-one

To a solution of 5-(2-(2-aminopyridin-3-yl)-3-(4-(((tert-butyldimethylsilyl)oxy)methyl)phenyl)-3H-imidazo[4,5-b]pyridin-5-yl)-1-(fluoromethyl)pyridin-2(1H)-one (570 mg, 1.02 mmol) in THF (10 mL) was added TBAF (0.8 mL, 1 M in THF). The mixture was stirred at 25° C. for 1 hr. The reaction mixture was quenched with H₂O (20 mL) at 25° C. and extracted with EtOAc (10 mL×3). The combined organic layers were washed with brine (20 mL×3), dried over Na₂SO₄, filtered and concentrated under reduced pressure. 5-(2-(2-Aminopyridin-3-yl)-3-(4-(hydroxymethyl)phenyl)-3H-imidazo[4,5-b]pyridin-5-yl)-1-(fluoromethyl)pyridin-2(1H)-one (440 mg) was obtained as a brown solid. MS: m/z=442.9 [M+H]⁺. ¹H NMR (400 MHz, Dimethylsulfoxide-d₆) δ 8.60 (d, J=2.4 Hz, 1H), 8.26 (d, J=8.4 Hz, 1H), 8.16 (dd, J=9.6, 2.4 Hz, 1H), 7.99 (dd, J=4.8, 2.0 Hz, 1H), 7.85 (d, J=8.4 Hz, 1H), 7.50-7.46 (m, 2H), 7.45-7.41 (m, 2H), 7.22-7.16 (m, 1H), 6.99-6.92 (m, 2H), 6.61 (d, J=10.0 Hz, 1H), 6.41 (dd, J=7.6, 4.8 Hz, 1H), 6.03 (d, J=50.8 Hz, 2H), 5.38-5.33 (m, 1H), 4.62-4.57 (m, 2H). ¹⁹F NMR (400 MHz, Dimethylsulfoxide-d₆) δ-172.572.

### Step 4: 5-(2-(2-Aminopyridin-3-yl)-3-(4-(chloromethyl)phenyl)-3H-imidazo[4,5-b]pyridin-5-yl)-1-(fluoromethyl)pyridin-2(1H)-one

To a solution of 5-(2-(2-aminopyridin-3-yl)-3-(4-(hydroxymethyl)phenyl)-3H-imidazo[4,5-b]pyridin-5-yl)-1-(fluoromethyl)pyridin-2(1H)-one (440 mg, 994 μmol) in CH₂Cl₂ (5 mL) was added SOCl₂ (355 mg, 2.98 mmol). The mixture was stirred at 40° C. for 0.5 hr. The reaction was concentrated under reduced pressure to give 5-(2-(2-aminopyridin-3-yl)-3-(4-(chloromethyl)phenyl)-3H-imidazo[4,5-b]pyridin-5-yl)-1-(fluoromethyl)pyridin-2(1H)-one (Intermediate 297, 458 mg, HCl salt) as a yellow solid. MS: m/z=461.0, 462.7 [M+H]⁺.

### Intermediate 280: 4-Bromo-2-(methyl-d₃)-2H-1,2,3-triazole

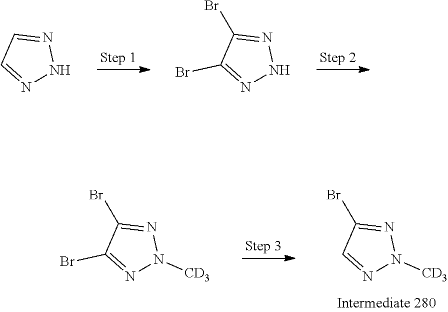 [SMILES: c1cn[nH]n1 ; Brc1n[nH]nc1Br ; [2H]C([2H])([2H])n1nc(Br)c(Br)n1 ; [2H]C([2H])([2H])n1ncc(Br)n1]

### Step 1: 4,5-Dibromo-2H-1,2,3-triazole

To a solution of 2H-1,2,3-triazole (25 g, 362 mmol) in H₂O (300 mL) was added dropwise Br₂ (75.2 g, 471 mmol) at 0° C. under N₂ atmosphere. The mixture was stirred at 25° C. for 16 hr under N₂ atmosphere. The reaction mixture was filtered. After triturated with MeOH (100 mL) at 25° C. for 10 min, 4,5-dibromo-2H-1,2,3-triazole (48 g, yield: 58%) was obtained as a white solid. MS: m/z=226.0, 228.0, 230.0 [M+H]⁺.

### Step 2: 4,5-Dibromo-2-(methyl-d₃)-2H-1,2,3-triazole

To a solution of 4,5-dibromo-2H-1,2,3-triazole (20 g, 88.2 mmol) and K₂CO₃ (24.4 g, 176 mmol) in DMF (100 mL) was added dropwise CD₃I (25 g, 176 mmol) at 0° C. The mixture was stirred at 25° C. for 16 hr. The reaction mixture was quenched with H₂O (200 mL) at 25° C. and extracted with CH₂Cl₂ (200 mL×3). The combined organic layers were washed with brine (200 mL×3), dried over Na₂SO₄, filtered and concentrated under reduced pressure. After purified by silica gel flash chromatography (Eluent of 0% ˜30% EtOAc in petroleum ether), 4,5-dibromo-2-(methyl-d₃)-2H-1,2,3-triazole (10 g, yield: 47%) was obtained as a colorless oil. MS: m/z=243.0, 245.0, 247.0 [M+H]⁺.

### Step 3: 4-Bromo-2-(methyl-d₃)-2H-1,2,3-triazole

To a solution of 4,5-dibromo-2-(methyl-d₃)-2H-1,2,3-triazole (10 g, 41 mmol) in THF (100 mL) was added dropwise n-BuLi (5.25 g, 82 mmol, 2.5 M in THF) at −78° C. The mixture was stirred at −78° C. for 1 hr. The reaction mixture was quenched with H₂O (200 mL) at 25° C. and extracted with CH₂Cl₂ (200 mL×3). The combined organic layers were washed with brine (200 mL×3), dried over Na₂SO₄, filtered and concentrated under reduced pressure. 4-Bromo-2-(methyl-d)-2H-1,2,3-triazole (Intermediate 280, 3.6 g, yield: 53%) was obtained as a yellow oil. ¹H NMR (400 MHz, Dimethylsulfoxide-d₆) δ 7.94 (s, 1H).

### Intermediate 281: 3-(3-(4-(Chloromethyl)phenyl)-5-(2-(methyl-d₃)-2H-1,2,3-triazol-4-yl)-3H-imidazo[4,5-b]pyridin-2-yl)pyridin-2-amine

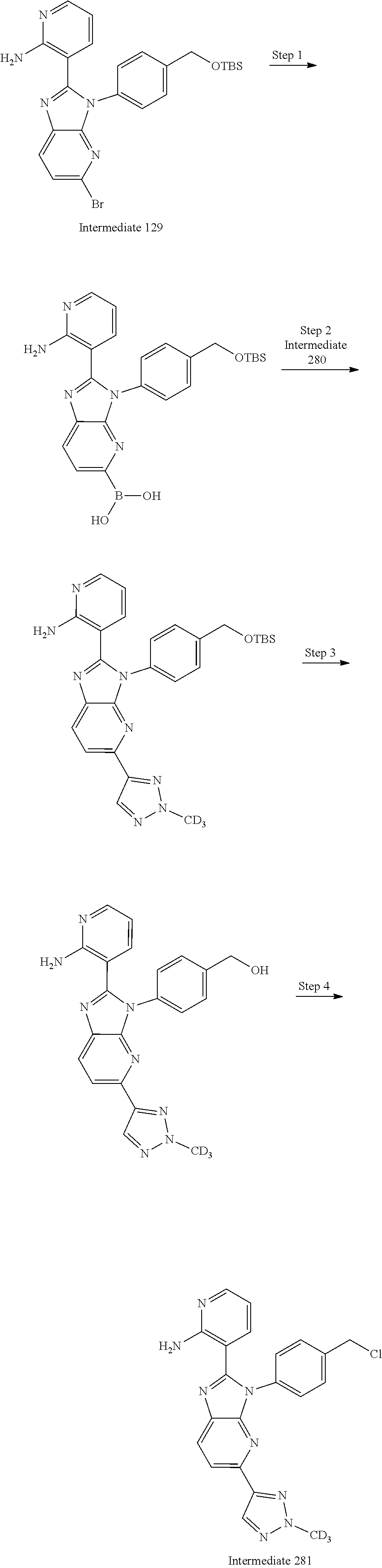 [SMILES: CC(C)(C)[Si](C)(C)OCc1ccc(-n2c(-c3cccnc3N)nc3ccc(Br)nc32)cc1 ; CC(C)(C)[Si](C)(C)OCc1ccc(-n2c(-c3cccnc3N)nc3ccc(B(O)O)nc32)cc1 ; [2H]C([2H])([2H])n1ncc(-c2ccc3nc(-c4cccnc4N)n(-c4ccc(CO[Si](C)(C)C(C)(C)C)cc4)c3n2)n1 ; [2H]C([2H])([2H])n1ncc(-c2ccc3nc(-c4cccnc4N)n(-c4ccc(CO)cc4)c3n2)n1 ; [2H]C([2H])([2H])n1ncc(-c2ccc3nc(-c4cccnc4N)n(-c4ccc(CCl)cc4)c3n2)n1]

### Step 1: (2-(2-Aminopyridin-3-yl)-3-(4-(((tert-butyldimethylsilyl)oxy)methyl)phenyl)-3H-imidazo[4,5-b]pyridin-5-yl)boronic acid

A mixture of Intermediate 129 (11 g, 21.6 mmol), 4,4,4′,4′,5,5,5′,5′-octamethyl-2,2′-bi(1,3,2-dioxaborolane) (10.9 g, 43.1 mmol), KOAc (6.34 g, 64.6 mmol) and Pd(dppf)Cl₂ (1.58 g, 2.15 mmol) in 1,4-dioxane (100 mL) was degassed and purged with N₂ three times. The mixture was stirred at 80° C. for 4 hr under N₂ atmosphere. (2-(2-aminopyridin-3-yl)-3-(4-(((tert-butyldimethylsilyl)oxy)methyl)phenyl)-3H-imidazo[4,5-b]pyridin-5-yl)boronic acid was obtained as a black liquid, the mixture was used for next step directly and without work-up and purification. MS: m/z=476.1 [M+H]⁺.

### Step 2: 3-(3-(4-(((tert-Butyldimethylsilyl)oxy)methyl)phenyl)-5-(2-(methyl-d₃)-2H-1,2,3-triazol-4-yl)-3H-imidazo[4,5-b]pyridin-2-yl)pyridin-2-amine

A mixture of (2-(2-aminopyridin-3-yl)-3-(4-(((tert-butyldimethylsilyl)oxy)methyl)phenyl)-3H-imidazo[4,5-b]pyridin-5-yl)boronic acid (10 g, 21 mmol), Intermediate 280 (3.47 g, 21 mmol), Pd(dppf)Cl₂ (1.54 g, 2.1 mmol) and Cs₂CO₃ (20.6 g, 63.1 mmol) in 1,4-dioxane (100 mL) and H₂O (20 mL) was degassed and purged with N₂ three times. The mixture was stirred at 80° C. for 16 hr under N₂ atmosphere. The reaction mixture was quenched with H₂O (200 mL) and extracted with CH₂Cl₂ (200 mL×2). The combined organic layers were washed with brine (200 mL), dried over Na₂SO₄, filtered and concentrated under reduced pressure. After purified by silica gel flash chromatography (Eluent of 10%˜45% EtOAc in petroleum ether), 3-(3-(4-(((tert-butyldimethylsilyl)oxy)methyl)phenyl)-5-(2-(methyl-d₃)-2H-1,2,3-triazol-4-yl)-3H-imidazo[4,5-b]pyridin-2-yl)pyridin-2-amine (200 mg, yield: 4.1% for two steps) was obtained as a brown solid. MS: m/z=516.1 [M+H]+. ¹H NMR (400 MHz, Dimethylsulfoxide-d₆) δ 8.27 (d, J=8.4 Hz, 1H), 8.05-7.87 (m, 3H), 7.51-7.44 (m, 4H), 7.18 (dd, J=7.6, 1.6 Hz, 1H), 6.97 (s, 2H), 6.38 (dd, J=7.6, 4.8 Hz, 1H), 4.82 (s, 2H), 0.93 (s, 9H), 0.11 (s, 6H).

### Step 3: (4-(2-(2-Aminopyridin-3-yl)-5-(2-(methyl-d₃)-2H-1,2,3-triazol-4-yl)-3H-imidazo[4,5-b]pyridin-3-yl)phenyl)methanol

To a solution of 3-(3-(4-(((tert-butyldimethylsilyl)oxy)methyl)phenyl)-5-(2-(methyl-d₃)-2H-1,2,3-triazol-4-yl)-3H-imidazo[4,5-b]pyridin-2-yl)pyridin-2-amine (500 mg, 970 μmol) in THE (5 mL) was added TBAF (507 mg, 1.94 mmol, 1 M in THF). The mixture was stirred at 25° C. for 1 hr. The reaction mixture was quenched with H₂O (20 mL) at 25° C. and extracted with CH₂Cl₂ (20 mL×3). The combined organic layers were washed with brine (20 mL×3), dried over Na₂SO₄, filtered and concentrated under reduced pressure. (4-(2-(2-Aminopyridin-3-yl)-5-(2-(methyl-d₃)-2H-1,2,3-triazol-4-yl)-3H-imidazo[4,5-b]pyridin-3-yl)phenyl)methanol (380 mg, yield: 84%) was obtained as a brown solid. MS: m/z=402.2 [M+H]⁺. ¹H NMR (400 MHz, Dimethylsulfoxide-d₆) S8.27 (d, J=8.4 Hz, 1H), 8.04 (s, 1H), 8.00 (dd, J=4.8, 1.6 Hz, 1H), 7.93 (d, J=8.4 Hz, 1H), 7.52-7.40 (m, 4H), 7.25-7.16 (m, 1H), 6.93 (s, 2H), 6.41 (dd, =7.6, 4.8 Hz, 1H), 5.37 (t, J=5.6 Hz, 1H), 4.60 (d, J=5.6 Hz, 2H).

### Step 4: 3-(3-(4-(Chloromethyl)phenyl)-5-(2-(methyl-d₃)-2H-1,2,3-triazol-4-yl)-3H-imidazo[4,5-b]pyridin-2-yl)pyridin-2-amine

To a solution of (4-(2-(2-aminopyridin-3-yl)-5-(2-(methyl-d₃)-2H-1,2,3-triazol-4-yl)-3H-imidazo[4,5-b]pyridin-3-yl)phenyl)methanol (380 mg, 947 μmol) in CH₂Cl₂ (5 mL) was added SOCl₂ (338 mg, 2.84 mmol). The mixture was stirred at 40° C. for 0.5 hr. The reaction was concentrated under reduced pressure to give 3-(3-(4-(chloromethyl)phenyl)-5-(2-(methyl-d₃)-2H-1,2,3-triazol-4-yl)-3H-imidazo[4,5-b]pyridin-2-yl)pyridin-2-amine (Intermediate 281, 420 mg, HCl salt) as a brown solid. MS: m/z=420.2 [M+H]r.

### Intermediate 282 & 283: 4,5-Dibromo-2-ethyl-2H-1,2,3-triazole & 4,5-Dibromo-1-ethyl-1H-1,2,3-triazole

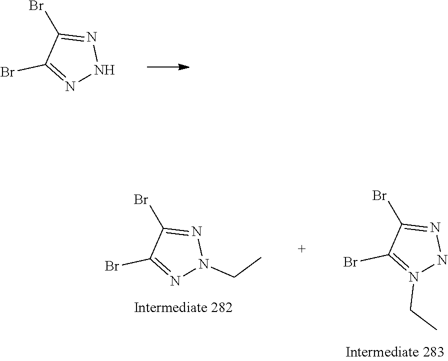

To a solution of 4,5-dibromo-2H-triazole (2 g, 8.82 mmol) in DMF (20 mL) were added K₂CO₃ (1.83 g, 13.2 mmol) and iodoethane (1.38 g, 8.82 mmol, 705 μL) at 20° C. The reaction mixture was stirred at 20° C. for 12 hr. The reaction mixture was diluted with H₂O (100 mL) and extracted with EtOAc (100 mL). The organic layer was separated, dried over anhydrous Na₂SO₄, filtered and concentrated under vacuum. After purified by silica gel flash chromatography (EtOAc in petroleum ether=0-5%), 4,5-dibromo-2-ethyl-2H-1,2,3-triazole (Intermediate 282, 3.1 g, yield: 69%) was obtained as a yellow oil. ¹H NMR (400 MHz, Dimethylsulfoxide-d₆) δ 4.45 (q, J=7.2 Hz, 2H), 1.43 (t, J=7.2 Hz, 3H). 4,5-Dibromo-1-ethyl-1H-1,2,3-triazole (Intermediate 283, 780 mg, yield: 17%) was obtained as a white solid. ¹H NMR (400 MHz, Dimethylsulfoxide-d₆) δ 4.42 (q, J=7.2 Hz, 2H), 1.41 (t, J=7.2 Hz, 3H).

### Intermediate 284: 4-Bromo-2-ethyl-2H-1,2,3-triazole

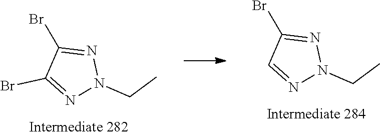

To a mixture of Intermediate 282 (1 g, 3.92 mmol) in THF (10 mL) was added n-BuLi (2.5M in hexane, 1.73 mL) at −70° C. under N₂. The mixture was stirred at −70° C. for 1 hr under N₂. The reaction mixture was quenched with H₂O (20 mL) at −70° C. under N₂ and extracted with EtOAc (20 mL×3). The combined organic layers were washed with brine (10 mL), dried over anhydrous Na₂SO₄, filtered and concentrated under reduced pressure. 4-Bromo-2-ethyl-2H-1,2,3-triazole (Intermediate 284, 600 mg, 3.41 mmol, yield: 87%) was obtained as a yellow oil. MS: m/z=175.7, 177.7 [M+H]⁺. ¹H NMR (400 MHz, Dimethylsulfoxide-d₆) δ 7.95 (s, 1H), 4.44 (q, J=7.2 Hz, 2H), 1.43 (t, J=7.2 Hz, 3H).

### Intermediate 285: 3-(3-(4-(Chloromethyl)phenyl)-5-(2-ethyl-2H-1,2,3-triazol-4-yl)-3H-imidazo[4,5-b]pyridin-2-yl)pyridin-2-amine

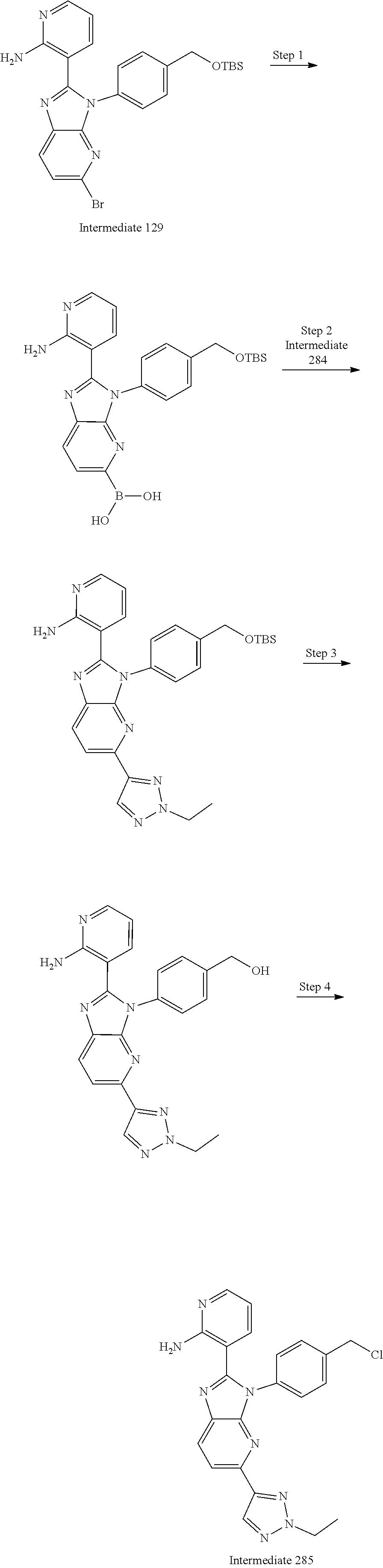 [SMILES: CC(C)(C)[Si](C)(C)OCc1ccc(-n2c(-c3cccnc3N)nc3ccc(Br)nc32)cc1 ; CC(C)(C)[Si](C)(C)OCc1ccc(-n2c(-c3cccnc3N)nc3ccc(B(O)O)nc32)cc1 ; CCn1ncc(-c2ccc3nc(-c4cccnc4N)n(-c4ccc(CO[Si](C)(C)C(C)(C)C)cc4)c3n2)n1 ; CCn1ncc(-c2ccc3nc(-c4cccnc4N)n(-c4ccc(CO)cc4)c3n2)n1 ; CCn1ncc(-c2ccc3nc(-c4cccnc4N)n(-c4ccc(CCl)cc4)c3n2)n1]

### Step 1: (2-(2-Aminopyridin-3-yl)-3-(4-(((tert-butyldimethylsilyl)oxy)methyl)phenyl)-3H-imidazo[4,5-b]pyridin-5-yl)boronic acid

A mixture of Intermediate 129 (320 mg, 0.63 mmol), 4,4,4′,4′,5,5,5′,5′-octamethyl-2,2′-bi(1,3,2-dioxaborolane) (318 mg, 1.25 mmol), KOAc (184 mg, 1.88 mmol) and Pd(dppf)Cl₂ (46 mg, 63 μmol) in 1,4-dioxane (5 mL) was degassed and purged with N₂ three times. The mixture was stirred at 80° C. for 4 hr under N₂ atmosphere. (2-(2-Aminopyridin-3-yl)-3-(4-(((tert-butyldimethylsilyl)oxy)methyl)phenyl)-3H-imidazo[4,5-b]pyridin-5-yl)boronic acid was obtained as a black liquid, which was used in the next step without work-up and purification. MS: m/z=476.1 [M+H]⁺.

### Step 2: 3-(3-(4-(((tert-Butyldimethylsilyl)oxy)methyl)phenyl)-5-(2-ethyl-2H-1,2,3-triazol-4-yl)-3H-imidazo[4,5-b]pyridin-2-yl)pyridin-2-amine

To a mixture of (2-(2-aminopyridin-3-yl)-3-(4-(((lert-butyldimethylsilyl)oxy)methyl)phenyl)-3H-imidazo[4,5-b]pyridin-5-yl)boronic acid (300 mg, 631 μmol) and Intermediate 284 (122 mg, 694 μmol) in 1,4-dioxane (6 mL) and H₂O (2 mL) were added Cs₂CO₃ (617 mg, 1.89 mmol) and Pd(dppf)Cl₂ (46.2 mg, 63.1 μmol). The mixture was stirred at 100° C. for 2 hr under N₂. The mixture was diluted with H₂O (20 mL) and extracted with EtOAc (20 mL×2). The combined organic layers were washed with brine (20 mL), dried over anhydrous Na₂SO₄, filtered and concentrated. After purified by silica gel flash chromatography (EtOAc in petroleum ether=0-50%), 3-(3-(4-(((tert-butyldimethylsilyl)oxy)methyl)phenyl)-5-(2-ethyl-2H-1,2,3-triazol-4-yl)-3H-imidazo[4,5-b]pyridin-2-yl)pyridin-2-amine (260 mg, 494 μmol, yield: 78% for two steps) was obtained as a yellow solid. MS: m/z=527.3 [M+H]⁺. ¹H NMR (400 MHz, Dimethylsulfoxide-d₆) δ 8.27 (d, J=8.4 Hz, 1H), 8.03 (s, 1H), 7.99 (dd, J=4.8, 2.0 Hz, 1H), 7.94 (d, J=8.0 Hz, 1H), 7.50-7.43 (m, 4H), 7.18 (dd, J=7.6, 2.0 Hz, 1H), 6.97 (s, 2H), 6.38 (dd, J=8.0, 5.2 Hz, 1H), 4.81 (s, 2H), 4.50 (q, J=7.2 Hz, 2H), 1.49 (t, J=7.2 Hz, 3H), 0.92 (s, 9H), 0.11 (s, 6H).

### Step 3: (4-(2-(2-Aminopyridin-3-yl)-5-(2-ethyl-2H-1,2,3-triazol-4-yl)-3H-imidazo[4,5-b]pyridin-3-yl)phenyl)methanol

To a mixture of 3-(3-(4-(((tert-butyldimethylsilyl)oxy)methyl)phenyl)-5-(2-ethyl-2H-1,2,3-triazol-4-yl)-3H-imidazo[4,5-b]pyridin-2-yl)pyridin-2-amine (210 mg, 399 μmol) in THF (5 mL) was added TBAF (1 M in THF, 598 μL), the mixture was stirred at 20° C. for 1 hr. The mixture was diluted with H₂O (10 mL) and extracted with EtOAc (10 mL×2). The combined organic layers were washed with brine (10 mL), dried over anhydrous Na₂SO₄, filtered and concentrated, (4-(2-(2-aminopyridin-3-yl)-5-(2-ethyl-2H-1,2,3-triazol-4-yl)-3H-imidazo[4,5-b]pyridin-3-yl)phenyl)methanol (164 mg, 398 μmol, yield: 100%) was obtained as a yellow solid. MS: m/z=413.2 [M+H]⁺. ¹H NMR (400 MHz, Dimethylsulfoxide-d₆) δ 8.27 (d, J=8.0 Hz, 1H), 8.04 (s, 1H), 7.99 (dd, J=4.8, 2.0 Hz, 1H), 7.94 (d, J=8.4 Hz, 1H), 7.51-7.40 (m, 4H), 7.21 (dd, J=7.6, 2.0 Hz, 1H), 6.92 (s, 2H), 6.41 (dd, J=7.6, 4.8 Hz, 1H), 5.37 (t, J=5.6 Hz, 1H), 4.60 (d, J=5.6 Hz, 2H), 4.50 (q, J=7.6 Hz, 2H), 1.49 (t, 1=7.6 Hz, 3H).

### Step 4: 3-(3-(4-(Chloromethyl)phenyl)-5-(2-ethyl-2H-1,2,3-triazol-4-yl)-3H-imidazo[4,5-b]pyridin-2-yl)pyridin-2-amine

To a mixture of (4-(2-(2-aminopyridin-3-yl)-5-(2-ethyl-2H-1,2,3-triazol-4-yl)-3H-imidazo[4,5-b]pyridin-3-yl)phenyl)methanol (164 mg, 398 μmol) in CH₂Cl₂ (2 mL) was added SOCl₂ (86.6 μL, 1.19 mmol), the mixture was stirred at 20° C. for 1 hr. The mixture was concentrated, 3-(3-(4-(chloromethyl)phenyl)-5-(2-ethyl-2H-1,2,3-triazol-4-yl)-3H-imidazo[4,5-b]pyridin-2-yl)pyridin-2-amine (Intermediate 285, 171 mg, 397 μmol, yield: 100%) was obtained as a yellow solid. MS: m/z=431.2, 433.1 [M+H]⁺.

### Intermediate 286 & 287: 4,5-Dibromo-2-isopropyl-2H-1,2,3-triazole & 4,5-Dibromo-1-isopropyl-1H-1,2,3-triazole

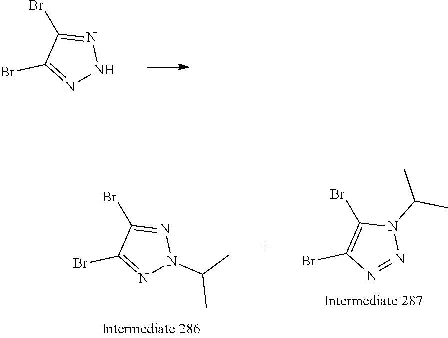

To a solution of 4,5-dibromo-2H-1,2,3-triazole (2 g, 8.82 mmol, 1 eq) in DMF (10 mL) were added K₂CO₃ (1.83 g, 13.2 mmol) and 2-iodopropane (1.50 g, 8.82 mmol, 880 μL) at 20° C. The reaction mixture was stirred at 20° C. for 12 hr. The mixture was diluted with H₂O (50 mL) and extracted with EtOAc (50 mL). The organic layer was separated, dried over anhydrous Na₂SO₄ and concentrated under reduced pressure. After purified by silica gel flash chromatography (0-10% EtOAc in petroleum ether), 4,5-dibromo-2-isopropyl-2H-1,2,3-triazole (Intermediate 286, 1.7 g, yield: 71.7%) was obtained as a colorless liquid. ¹H NMR (400 MHz, Chloroform-d) 64.82-4.71 (m, 1H), 1.55 (d, J=6.8 Hz, 6H). 4,5-Dibromo-1-isopropyl-1H-1,2,3-triazole (Intermediate 287, 320 mg, yield: 13.5%) was obtained as a colorless liquid. ¹H NMR (400 MHz, Chloroform-d) δ 4.83-4.71 (m, 1H), 1.64 (d, J=6.8 Hz, 6H).

### Intermediate 288: 4-Bromo-2-isopropyl-2H-1,2,3-triazole

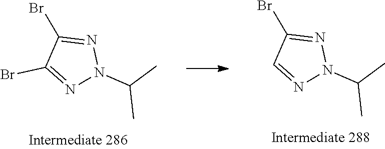

To a solution of Intermediate 286 (1 g, 3.72 mmol) in THF (9 mL) was added n-BuLi (2.5 M in hexane, 1.56 mL) at −70° C. The reaction mixture was stirred at −70° C. for 0.5 hr under N₂. The reaction mixture was quenched with saturated aq. NH₄Cl (30 mL) dropwise at −70° C. under N₂. The mixture was then diluted with H₂O (20 mL) and extracted with EtOAc (30 mL×2). The combined organic layers were washed with brine (30 mL), dried over anhydrous Na₂SO₄, filtered and concentrated under reduced pressure. 4-Bromo-2-isopropyl-2H-1,2,3-triazole (Intermediate 288, 590 mg, 3.10 mmol, yield: 83.5%) was obtained as a colorless liquid, which was used in the next step directly. MS: m/z=190.0, 192.0[M+H]⁺. ¹H NMR (400 MHz, Chloroform-d) δ=7.51 (s, 1H), 4.87-4.73 (m, 1H), 1.56 (d, J=6.8 Hz, 6H).

### Intermediate 289: 3-(3-(4-(Chloromethyl)phenyl)-5-(2-isopropyl-2H-1,2,3-triazol-4-yl)-3H-imidazo[4,5-b]pyridin-2-yl)pyridin-2-amine

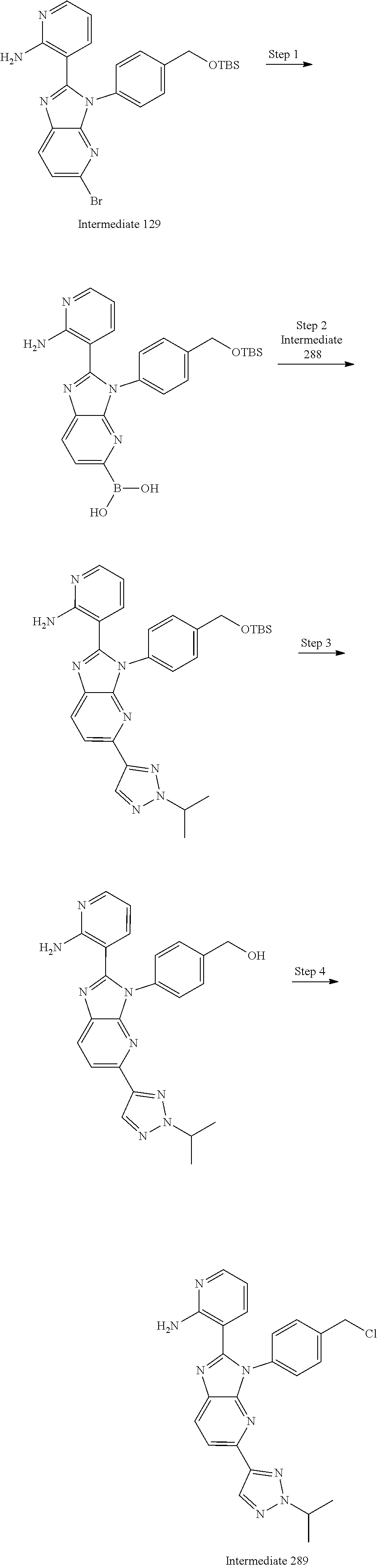 [SMILES: CC(C)(C)[Si](C)(C)OCc1ccc(-n2c(-c3cccnc3N)nc3ccc(Br)nc32)cc1 ; CC(C)(C)[Si](C)(C)OCc1ccc(-n2c(-c3cccnc3N)nc3ccc(B(O)O)nc32)cc1 ; CC(C)n1ncc(-c2ccc3nc(-c4cccnc4N)n(-c4ccc(CO[Si](C)(C)C(C)(C)C)cc4)c3n2)n1 ; CC(C)n1ncc(-c2ccc3nc(-c4cccnc4N)n(-c4ccc(CO)cc4)c3n2)n1 ; CC(C)n1ncc(-c2ccc3nc(-c4cccnc4N)n(-c4ccc(CCl)cc4)c3n2)n1]

### Step 1: (2-(2-Aminopyridin-3-yl)-3-(4-(((tert-butyldimethylsilyl)oxy)methyl)phenyl)-3H-imidazo[4,5-b]pyridin-5-yl)boronic acid

A mixture of Intermediate 129 (1 g, 1.96 mmol), 4,4,4′,4′,5,5,5′,5′-octamethyl-2,2′-bi(1,3,2-dioxaborolane) (995 mg, 3.92 mmol), KOAc (577 mg, 5.88 mmol) and Pd(dppf)Cl₂ (143 mg, 196 μmol) in 1,4-dioxane (10 mL) was degassed and purged with N₂ three times. The mixture was stirred at 80° C. for 4 hr under N₂ atmosphere. (2-(2-Aminopyridin-3-yl)-3-(4-(((tert-butyldimethylsilyl)oxy)methyl)phenyl)-3H-imidazo[4,5-b]pyridin-5-yl)boronic acid was obtained as a black liquid, which was used in the next step without work-up and purification. MS: m/z=476.1 [M+H]⁺.

### Step 2: 3-(3-(4-(((tert-Butyldimethylsilyl)oxy)methyl)phenyl)-5-(2-isopropyl-2H-1,2,3-triazol-4-yl)-3H-imidazo[4,5-b]pyridin-2-yl)pyridin-2-amine

To a solution of (2-(2-aminopyridin-3-yl)-3-(4-(((tert-butyldimethylsilyl)oxy)methyl)phenyl)-3H-imidazo[4,5-b]pyridin-5-yl)boronic acid (373 mg, 784 μmol) and 4-bromo-2-isopropyl-triazole (179 mg, 940 μmol) in 1,4-dioxane (12.5 mL) and H₂O (2.5 mL) were added Pd(dppf)Cl₂ (57.3 mg, 78.4 μmol) and Cs₂CO₃ (766 mg, 2.35 mmol) at 20° C. The mixture was stirred at 100° C. for 2 hr under N₂. The mixture was diluted with H₂O (20 mL) and extracted with EtOAc (20 mL×2). The combined organic layers were washed with brine (20 mL), dried over anhydrous Na₂SO₄, filtered and concentrated. After purified by silica gel flash chromatography (40-80% EtOAc in petroleum ether), 3-(3-(4-(((tert-butyldimethylsilyl)oxy)methyl)phenyl)-5-(2-isopropyl-2H-1,2,3-triazol-4-yl)-3H-imidazo[4,5-b]pyridin-2-yl)pyridin-2-amine (180 mg, 333 μmol, yield: 42.5% for two steps) was obtained as a yellow oil. MS: m/z=541.3 [M+H]⁺. ¹H NMR (400 MHz, Dimethylsulfoxide-d₆) δ 8.27 (d, J=8.4 Hz, 1H), 8.03-7.93 (m, 3H), 7.51-7.41 (m, 4H), 7.18 (dd, J=7.6, 2.0, Hz, 1H), 6.98 (s, 2H), 6.38 (dd, J=7.6, 4.8 Hz, 1H), 4.93-4.79 (m, 3H), 1.53 (d, J=6.8 Hz, 6H), 0.92 (s, 9H), 0.11 (s, 6H).

### Step 3: (4-(2-(2-Aminopyridin-3-yl)-5-(2-isopropyl-2H-1,2,3-triazol-4-yl)-3H-imidazo[4,5-b]pyridin-3-yl)phenyl)methanol

To a solution of 3-(3-(4-(((tert-butyldimethylsilyl)oxy)methyl)phenyl)-5-(2-isopropyl-2H-1,2,3-triazol-4-yl)-3H-imidazo[4,5-b]pyridin-2-yl)pyridin-2-amine (180 mg, 333 μmol) was added TBAF (1 M in THF, 533 μL). The reaction mixture was stirred at 20° C. for 0.5 hr. The mixture was diluted with H₂O (10 mL) and extracted with EtOAc (10 mL×2). The combined organic layers were washed with brine (10 mL), dried over anhydrous Na₂SO₄, filtered and concentrated. (4-(2-(2-Aminopyridin-3-yl)-5-(2-isopropyl-2H-1,2,3-triazol-4-yl)-3H-imidazo[4,5-b]pyridin-3-yl)phenyl)methanol (140 mg, 328 μmol) was obtained as a brown oil, which was used in the next step directly. MS: m/z=427.3 [M+H]⁺. ¹H NMR (400 MHz, Dimethylsulfoxide-d₆) δ 8.26 (d, J=8.4 Hz, 1H), 8.10-7.92 (m, 3H), 7.55-7.37 (m, 4H), 7.20 (d, J=7.6 Hz, 1H), 6.93 (s, 2H), 6.52-6.35 (m, 1H), 5.37 (s, 1H), 4.97-4.82 (m, 1H), 4.60 (s, 2H), 1.53 (d, J=6.8 Hz, 6H).

### Step 4: 3-(3-(4-(Chloromethyl)phenyl)-5-(2-isopropyl-2H-1,2,3-triazol-4-yl)-3H-imidazo[4,5-b]pyridin-2-yl)pyridin-2-amine

To a solution of (4-(2-(2-aminopyridin-3-yl)-5-(2-isopropyl-2H-1,2,3-triazol-4-yl)-3H-imidazo[4,5-b]pyridin-3-yl)phenyl)methanol (140 mg, 328 μmol) in CH₂Cl₂ (5 mL) was added SOCl₂ (117 mg, 985 μmol, 71.5 IL). The reaction mixture was stirred at 20° C. for 1 hr. The reaction mixture was concentrated to give 3-(3-(4-(chloromethyl)phenyl)-5-(2-isopropyl-2H-1,2,3-triazol-4-yl)-3H-imidazo[4,5-b]pyridin-2-yl)pyridin-2-amine (Intermediate 289, 146 mg, 328 μmol) as a brown oil, which was used in the next step directly. MS: m/z=445.2 [M+H]⁺.

### Intermediate 290: 4-Bromo-1-isopropyl-1H-1,2,3-triazole

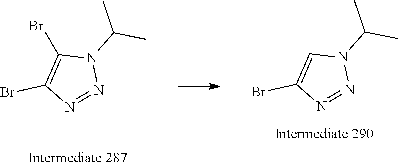

To a solution of Intermediate 287 (300 mg, 1.12 mmol) in THF (3 mL) was added n-BuLi (2.5 M in hexane, 469 μL) at −70° C. The reaction mixture was stirred at −70° C. for 0.5 hr under N₂. The reaction mixture was quenched with sat. aq. NH₄Cl (10 mL) dropwise at −70° C. under N₂. The mixture was diluted with H₂O (5 mL) and extracted with EtOAc (10 mL×2). The combined organic layers were washed with brine (20 mL), dried over anhydrous Na₂SO₄, filtered and concentrated under reduced pressure. 4-Bromo-1-isopropyl-1H-1,2,3-triazole (Intermediate 290, 210 mg, yield: 99%) was obtained as colorless liquid, which was used in the next step directly. MS: m/z=188.0, 190.0 [M+H]⁺. ¹H NMR (400 MHz, Chloroform-d) δ 7.56 (s, 1H), 4.87-4.78 (m, 1H), 1.58 (d, J=6.8 Hz, 6H).

### Intermediate 291: 3-(3-(4-(Chloromethyl)phenyl)-5-(1-isopropyl-1H-1,2,3-triazol-4-yl)-3H-imidazo[4,5-b]pyridin-2-yl)pyridin-2-amine

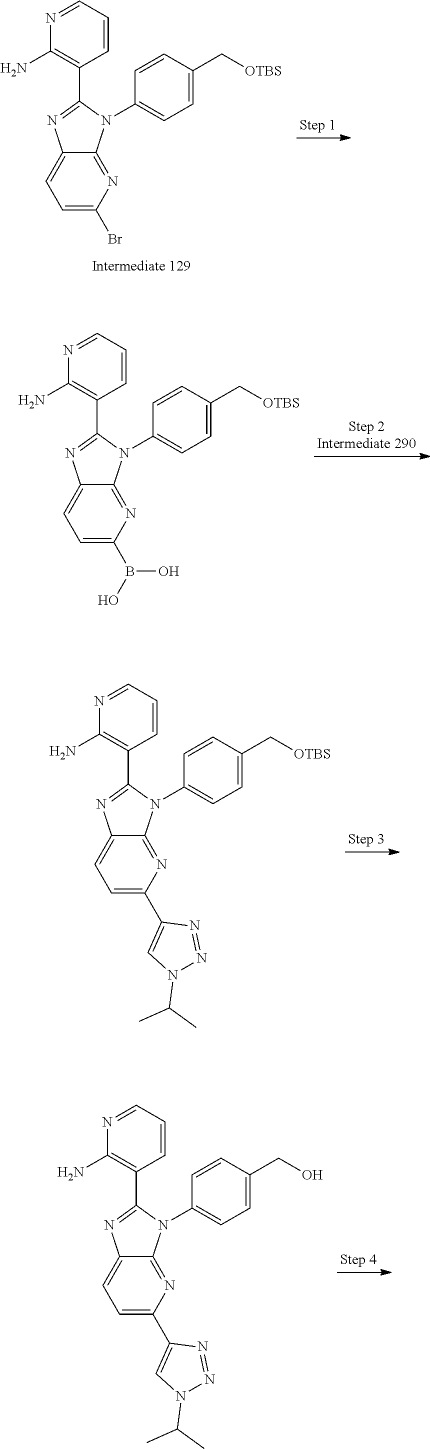 [SMILES: CC(C)(C)[Si](C)(C)OCc1ccc(-n2c(-c3cccnc3N)nc3ccc(Br)nc32)cc1 ; CC(C)(C)[Si](C)(C)OCc1ccc(-n2c(-c3cccnc3N)nc3ccc(B(O)O)nc32)cc1 ; CC(C)n1cc(-c2ccc3nc(-c4cccnc4N)n(-c4ccc(CO[Si](C)(C)C(C)(C)C)cc4)c3n2)nn1 ; CC(C)n1cc(-c2ccc3nc(-c4cccnc4N)n(-c4ccc(CO)cc4)c3n2)nn1]

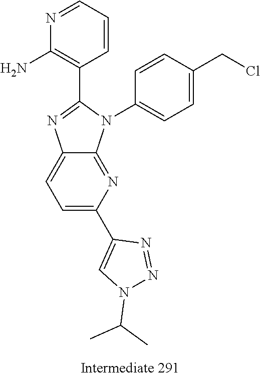 [SMILES: CC(C)n1cc(-c2ccc3nc(-c4cccnc4N)n(-c4ccc(CCl)cc4)c3n2)nn1]

### Step 1: (2-(2-Aminopyridin-3-yl)-3-(4-(((tert-butyldimethylsilyl)oxy)methyl)phenyl)-3H-imidazo[4,5-b]pyridin-5-yl)boronic acid

A mixture of Intermediate 129 (1 g, 1.96 mmol), 4,4,4′,4′,5,5,5′,5′-octamethyl-2,2′-bi(1,3,2-dioxaborolane) (995 mg, 3.92 mmol), KOAc (577 mg, 5.88 mmol) and Pd(dppf)Cl₂ (143 mg, 196 μmol) in 1,4-dioxane (10 mL) was degassed and purged with N₂ for 3 times. The mixture was stirred at 80° C. for 4 hr under N₂ atmosphere. (2-(2-Aminopyridin-3-yl)-3-(4-(((tert-butyldimethylsilyl)oxy)methyl)phenyl)-3H-imidazo[4,5-b]pyridin-5-yl)boronic acid was obtained as a black liquid, which was used in the next step without work-up and purification. MS: m/z=476.1 [M+H]⁺.

### Step 2: 3-(3-(4-(((tert-Butyldimethylsilyl)oxy)methyl)phenyl)-5-(1-isopropyl-1H-1,2,3-triazol-4-yl)-3H-imidazo[4,5-b]pyridin-2-yl)pyridin-2-amine

To a solution of (2-(2-aminopyridin-3-yl)-3-(4-(((tert-butyldimethylsilyl)oxy)methyl)phenyl)-3H-imidazo[4,5-b]pyridin-5-yl)boronic acid (373 mg, 784 μmol) and Intermediate 290 (194 mg, 1.02 mmol) in 1,4-dioxane (12.5 mL) and H₂O (2.5 mL) were added Pd(dppf)Cl₂ (57.3 mg, 78.4 μmol) and Cs₂CO₃ (766 mg, 2.35 mmol) at 20° C. The mixture was stirred at 100° C. for 2 hr under N₂. The mixture was diluted with H₂O (30 mL) and extracted with EtOAc (30 mL×2). The combined organic layers were washed with brine (30 mL), dried over anhydrous Na₂SO₄, filtered and concentrated. After purified by silica gel flash chromatography (Eluent of 40%˜80% EtOAc in petroleum ether), 3-(3-(4-(((tert-butyldimethylsilyl)oxy)methyl)phenyl)-5-(1-isopropyl-1H-1,2,3-triazol-4-yl)-3H-imidazo[4,5-b]pyridin-2-yl)pyridin-2-amine (280 mg, yield: 66% for two steps) was obtained as a brown solid. MS: m/z=541.3 [M+H]⁺. ¹H NMR (400 MHz, Dimethylsulfoxide-d₆) δ 8.39 (s, 1H), 8.27 (d, J=8.4 Hz, 1H), 8.09 (d, J=8.4 Hz, 1H), 7.98 (dd, J=4.8, 1.6 Hz, 1H), 7.49-7.43 (m, 4H), 7.15 (dd, J=7.6, 1.6, Hz, 1H), 6.97 (br s, 2H), 6.40-6.34 (m, 1H), 4.89-4.85 (m, 1H), 4.82 (s, 2H), 1.51 (d, J=6.8 Hz, 6H), 0.93 (s, 9H), 0.11 (s, 6H).

### Step 3: (4-(2-(2-Aminopyridin-3-yl)-5-(1-isopropyl-1H-1,2,3-triazol-4-yl)-3H-imidazo[4,5-b]pyridin-3-yl)phenyl)methanol

To a solution of 3-(3-(4-(((tert-butyldimethylsilyl)oxy)methyl)phenyl)-5-(1-isopropyl-1H-1,2,3-triazol-4-yl)-3H-imidazo[4,5-b]pyridin-2-yl)pyridin-2-amine (280 mg, 518 μmol) in THF (8 mL) was added TBAF (1 M in THF, 829 μL). The reaction mixture was stirred at 20° C. for 0.5 hr. The mixture was diluted with H₂O (10 mL) and extracted with EtOAc (10 mL×2). The combined organic layers were washed with brine (10 mL), dried over anhydrous Na₂SO₄, filtered and concentrated. (4-(2-(2-Aminopyridin-3-yl)-5-(1-isopropyl-1H-1,2,3-triazol-4-yl)-3H-imidazo[4,5-b]pyridin-3-yl)phenyl)methanol (220 mg) was obtained as yellow oil, which was used in the next step directly. MS: m/z=427.3 [M+H]⁺. ¹H NMR (400 MHz, Dimethylsulfoxide-d₆) δ 8.41 (s, 1H), 8.27 (d, J=8.4 Hz, 1H), 8.08 (d, J=8.4 Hz, 1H), 7.98 (dd, J=4.8, 1.6 Hz, 1H), 7.50-7.46 (m, 2H), 7.44-7.39 (m, 2H), 7.18 (dd, J=7.6, 1.6 Hz, 1H), 6.92 (br s, 2H), 6.40 (dd, J=7.6, 4.8 Hz, 1H), 4.93-4.84 (m, 1H), 4.60 (s, 2H), 1.52 (d, J=6.8 Hz, 6H).

### Step 4: 3-(3-(4-(Chloromethyl)phenyl)-5-(1-isopropyl-1H-1,2,3-triazol-4-yl)-3H-imidazo[4,5-b]pyridin-2-yl)pyridin-2-amine

To a solution of (4-(2-(2-aminopyridin-3-yl)-5-(1-isopropyl-1H-1,2,3-triazol-4-yl)-3H-imidazo[4,5-b]pyridin-3-yl)phenyl)methanol (220 mg, 516 μmol) in CH₂Cl₂ (8 mL) was added SOCl₂ (184 mg, 1.55 mmol, 112 μL). The reaction mixture was stirred at 20° C. for 1 hr. The reaction mixture was concentrated to give 3-(3-(4-(chloromethyl)phenyl)-5-(1-isopropyl-1H-1,2,3-triazol-4-yl)-3H-imidazo[4,5-b]pyridin-2-yl)pyridin-2-amine (Intermediate 291, 220 mg, HCl salt) as a brown oil, which was used in the next step directly. MS: m/z=445.2, 447.2 [M+H]⁺.

### Intermediate 292: 4-Bromo-2-methyl-5-(methyl-d₃)-2H-1,2,3-triazole

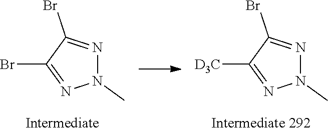

To a solution of 4,5-dibromo-2-methyl-2H-1,2,3-triazole (1 g, 4.2 mmol) in THF (10 mL) was degassed and purged with N₂ three times. n-BuLi (2.49 mL, 6.2 mmol, 2.5 M in n-hexane) was added at 0° C. and stirred at 0° C. for 0.5 hr under N₂ atmosphere. The mixture was added CD₃I (884 mg, 6.2 mmol) at 0° C. and stirred at 0° C. for 2 hr under N₂ atmosphere. The mixture was quenched with H₂O (100 mL) and extracted with CH₂Cl₂ (50 mL×2). The combined organic layers were washed with brine (50 mL×3), dried over Na₂SO₄, filtered and concentrated. 4-Bromo-2-methyl-5-(methyl-d₃)-2H-1,2,3-triazole (Intermediate 292, 550 mg, crude) was obtained as a yellow oil, which was used in the next step without purification.

### Intermediate 293: 3-(3-(4-(Chloromethyl)phenyl)-5-(2-methyl-5-(methyl-d₃)-2H-1,2,3-triazol-4-yl)-3H-imidazo[4,5-b]pyridin-2-yl)pyridin-2-amine

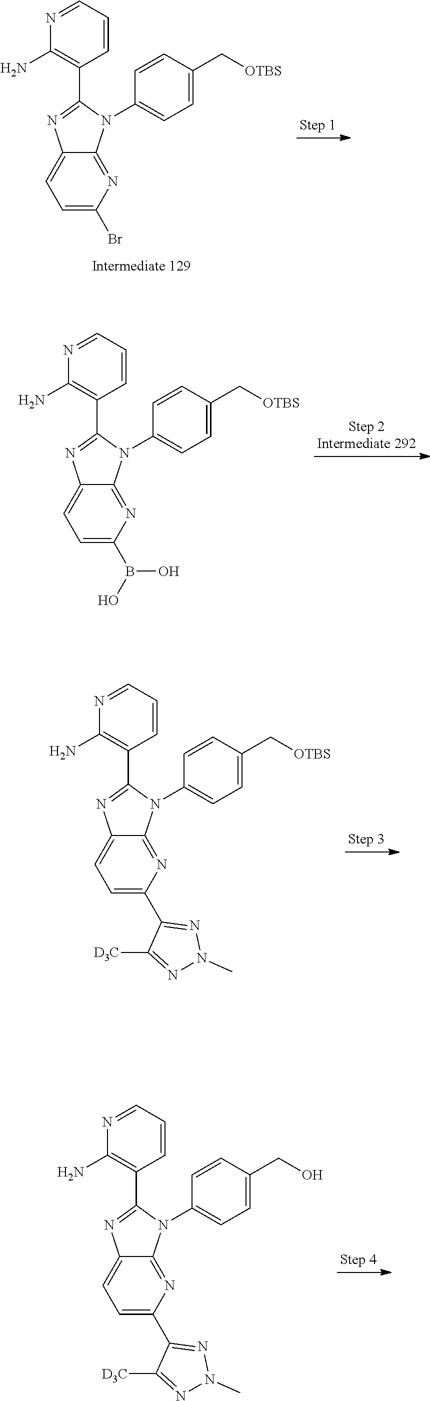 [SMILES: CC(C)(C)[Si](C)(C)OCc1ccc(-n2c(-c3cccnc3N)nc3ccc(Br)nc32)cc1 ; CC(C)(C)[Si](C)(C)OCc1ccc(-n2c(-c3cccnc3N)nc3ccc(B(O)O)nc32)cc1 ; [2H]C([2H])([2H])c1nn(C)nc1-c1ccc2nc(-c3cccnc3N)n(-c3ccc(CO[Si](C)(C)C(C)(C)C)cc3)c2n1 ; [2H]C([2H])([2H])c1nn(C)nc1-c1ccc2nc(-c3cccnc3N)n(-c3ccc(CO)cc3)c2n1]

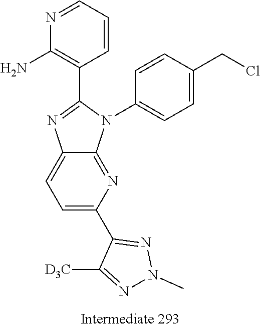 [SMILES: [2H]C([2H])([2H])c1nn(C)nc1-c1ccc2nc(-c3cccnc3N)n(-c3ccc(CCl)cc3)c2n1]

### Step 1: (2-(2-Aminopyridin-3-yl)-3-(4-(((tert-butyldimethylsilyl)oxy)methyl)phenyl)-3H-imidazo[4,5-b]pyridin-5-yl)boronic acid

A mixture of Intermediate 129 (1 g, 1.9 mmol), 4,4,4′,4′,5,5,5′,5′-octamethyl-2,2′-bi(1,3,2-dioxaborolane) (746 mg, 2.9 mmol), KOAc (577 mg, 5.9 mmol), and Pd(dppf)Cl₂ (80 mg, 98 μmol) in 1,4-dioxane (10 mL) was degassed and purged with N₂ three times. The mixture was stirred at 110° C. for 2 hr under N₂ atmosphere. (2-(2-Aminopyridin-3-yl)-3-(4-(((tert-butyldimethylsilyl)oxy)methyl)phenyl)-3H-imidazo[4,5-b]pyridin-5-yl)boronic acid was obtained as a black liquid, which was used in the next step without work-up and purification. MS: m/z=476.1 [M+H]⁺.

### Step 2: 3-(3-(4-(((Tert-butyldimethylsilyl)oxy)methyl)phenyl)-5-(2-methyl-5-(methyl-d₃)-2H-1,2,3-triazol-4-yl)-3H-imidazo[4,5-b]pyridin-2-yl)pyridin-2-amine

A mixture of (2-(2-aminopyridin-3-yl)-3-(4-(((tert-butyldimethylsilyl)oxy)methyl)phenyl)-3H-imidazo[4,5-b]pyridin-5-yl)boronic acid (500 mg, 1.0 mmol), Intermediate 292 (244 mg, 1.3 mmol), Pd(dppf)Cl₂ (86 mg, 105 μmol), and Cs₂CO₃ (1 g, 3.1 mmol) in 1,4-dioxane (5 mL) was degassed and purged with N₂ three times. The mixture was stirred at 95° C. for 12 hr under N₂ atmosphere. The reaction mixture was quenched with H₂O (30 mL) and extracted with CH₂Cl₂ (20 mL×2). The combined organic layers were washed with brine (50 mL), dried over Na₂SO₄, filtered and concentrated under reduced pressure. After purified by silica gel flash chromatography (Eluent of 0%˜25% EtOAc in petroleum ether), 3-(3-(4-(((tert-butyldimethylsilyl)oxy)methyl)phenyl)-5-(2-methyl-5-(methyl-d₃)-2H-1,2,3-triazol-4-yl)-3H-imidazo[4,5-b]pyridin-2-yl)pyridin-2-amine (200 mg, yield: 26% for three steps) was obtained as a yellow solid. MS: m/z=530.2 [M+H]⁺. D %: 3D %=99.9% ¹H NMR (400 MHz, Dimethylsulfoxide-d₆) δ 8.25 (d, J=8.4 Hz, 1H), 8.01-7.98 (m, 1H), 7.93-7.90 (m, 1H), 7.47-7.46 (m, 4H), 7.22 (dd, J=8.0, 1.6 Hz, 1H), 7.05-6.98 (m, 2H), 6.42-6.38 (m, 1H), 4.80 (s, 2H), 4.13 (s, 3H), 0.91 (s, 9H), 0.08 (s, 6H).

### Step 3: (4-(2-(2-Aminopyridin-3-yl)-5-(2-methyl-5-(methyl-d₃)-2H-1,2,3-triazol-4-yl)-3H-imidazo[4,5-b]pyridin-3-yl)phenyl)methanol

To a solution of 3-(3-(4-(((tert-butyldimethylsilyl)oxy)methyl)phenyl)-5-(2-methyl-5-(methyl-d₃)-2H-1,2,3-triazol-4-yl)-3H-imidazo[4,5-b]pyridin-2-yl)pyridin-2-amine (200 mg, 377 μmol) in THF (3 mL) was added TBAF (3.8 mL, 1 M in THF). The mixture was stirred at 25° C. for 2 hr. The reaction mixture was quenched with H₂O (5 mL) at 25° C. and extracted with CH₂Cl₂ (15 mL×2). The combined organic layers were washed with brine (30 mL×2), dried over Na₂SO₄, filtered and concentrated under reduced pressure. After triturated with EtOAc: petroleum ether=1:10 (11 mL) at 25° C. for 10 min, (4-(2-(2-aminopyridin-3-yl)-5-(2-methyl-5-(methyl-d₃)-2H-1,2,3-triazol-4-yl)-3H-imidazo[4,5-b]pyridin-3-yl)phenyl)methanol (150 mg, crude) was obtained as a yellow solid. MS: m/z=416.0 [M+H]⁺. ¹H NMR (400 MHz, Dimethylsulfoxide-d₆) δ 8.25 (d, J=8.4 Hz, 1H), 8.02-8.00 (m, 1H), 7.93-7.90 (m, 1H), 7.48-7.43 (m, 5H), 7.26-7.22 (m, 1H), 6.99-6.94 (m, 2H), 6.45-6.42 (m, 1H), 4.59-4.57 (m, 2H), 4.13 (s, 3H).

### Step 4: 3-(3-(4-(Chloromethyl)phenyl)-5-(2-methyl-5-(methyl-d₃)-2H-1,2,3-triazol-4-yl)-3H-imidazo[4,5-b]pyridin-2-yl)pyridin-2-amine

To a solution of (4-(2-(2-aminopyridin-3-yl)-5-(2-methyl-5-(methyl-d₃)-2H-1,2,3-triazol-4-yl)-3H-imidazo[4,5-b]pyridin-3-yl)phenyl)methanol (150 mg, 339 μmol) in CH₂Cl₂ (2 mL) was added SOCl₂ (262 μL, 3.61 mmol). The mixture was stirred at 25° C. for 1 hr. The reaction was concentrated under reduced pressure to give 3-(3-(4-(chloromethyl)phenyl)-5-(2-methyl-5-(methyl-d₃)-2H-1,2,3-triazol-4-yl)-3H-imidazo[4,5-b]pyridin-2-yl)pyridin-2-amine (Intermediate 293, 170 mg, crude, HCl salt) as a green solid which was used into next step directly. MS: m/z=434.2 [M+H]⁺.

### Intermediate 294: 3-(3-(4-(Chloromethyl)phenyl)-5-(2,5-dimethyl-2H-1,2,3-triazol-4-yl)-3H-imidazo[4,5-b]pyridin-2-yl)pyridin-2-amine

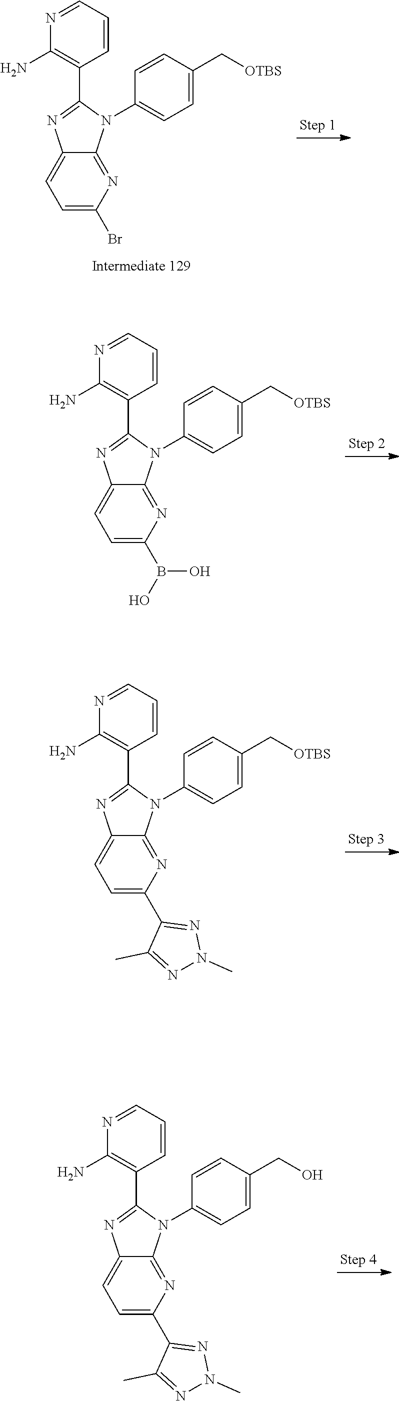 [SMILES: CC(C)(C)[Si](C)(C)OCc1ccc(-n2c(-c3cccnc3N)nc3ccc(Br)nc32)cc1 ; CC(C)(C)[Si](C)(C)OCc1ccc(-n2c(-c3cccnc3N)nc3ccc(B(O)O)nc32)cc1 ; Cc1nn(C)nc1-c1ccc2nc(-c3cccnc3N)n(-c3ccc(CO[Si](C)(C)C(C)(C)C)cc3)c2n1 ; Cc1nn(C)nc1-c1ccc2nc(-c3cccnc3N)n(-c3ccc(CO)cc3)c2n1]

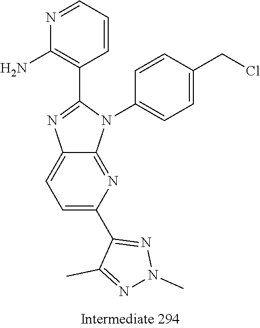 [SMILES: Cc1nn(C)nc1-c1ccc2nc(-c3cccnc3N)n(-c3ccc(CCl)cc3)c2n1]

### Step 1: (2-(2-Aminopyridin-3-yl)-3-(4-(((tert-butyldimethylsilyl)oxy)methyl)phenyl)-3H-imidazo[4,5-b]pyridin-5-yl)boronic acid

To a mixture of Intermediate 129 (500 mg, 979 μmol) in 1,4-dioxane (5 mL) were added potassium acetate (288 mg, 2.94 mmol) and 4,4,4′,4′,5,5,5′,5′-octamethyl-2,2′-bi(1,3,2-dioxaborolane) (497 mg, 1.96 mmol), and Pd(dppf)Cl₂ (71.7 mg, 97.9 μmol). The mixture was degassed and purged with N₂ three times and stirred at 85° C. for 2 hr under N₂. (2-(2-Aminopyridin-3-yl)-3-(4-(((tert-butyldimethylsilyl)oxy)methyl)phenyl)-3H-imidazo[4,5-b]pyridin-5-yl)boronic acid was obtained as a black liquid, which was used in the next step without work-up and purification. MS: m/z=476.2 [M+H]⁺.

### Step 2: 3-(3-(4-(((tert-Butyldimethylsilyl)oxy)methyl)phenyl)-5-(2,5-dimethyl-2H-1,2,3-triazol-4-yl)-3H-imidazo[4,5-b]pyridin-2-yl)pyridin-2-amine

To a mixture of (2-(2-aminopyridin-3-yl)-3-(4-(((peri-butyldimethylsilyl)oxy)methyl)phenyl)-3H-imidazo[4,5-b]pyridin-5-yl)boronic acid (465 mg, 978 μmol) and 4-bromo-2,5-dimethyl-2H-1,2,3-triazole (206 mg, 1.17 mmol) in 1,4-dioxane (10 mL) and H₂O (2 mL) were added Cs₂CO₃ (956 mg, 2.93 mmol) and Pd(dppf)Cl₂ (71.6 mg, 97.8 μmol). The mixture was degassed and purged with N₂ three times and stirred at 100° C. for 2 hr under N₂. The mixture was diluted with H₂O (20 mL) and extracted with EtOAc (50 mL×2). The combined organic layers were washed with brine (30 mL), dried over anhydrous Na₂SO₄, filtered and concentrated. After purified by silica gel flash chromatography (EtOAc in petroleum ether=0-50%), 3-(3-(4-(((tert-butyldimethylsilyl)oxy)methyl)phenyl)-5-(2,5-dimethyl-2H-1,2,3-triazol-4-yl)-3H-imidazo[4,5-b]pyridin-2-yl)pyridin-2-amine (380 mg, yield: 74% for two steps) was obtained as a brown solid. MS: m/z=527.3 [M+H]⁺. ¹H NMR (400 MHz, Dimethylsulfoxide-d₆) δ 8.25 (d, J=8.4 Hz, 1H), 8.01 (dd, J=4.8, 1.6 Hz, 1H), 7.91 (d, J=8.4 Hz, 1H), 7.49-7.42 (m, 4H), 7.22 (dd, J=7.6, 1.6 Hz, 1H), 7.02 (s, 2H), 6.40 (dd, J=7.6, 4.8 Hz, 1H), 4.80 (s, 2H), 4.13 (s, 3H), 2.36 (s, 3H), 0.91 (s, 9H), 0.08 (s, 6H).

### Step 3: (4-(2-(2-Aminopyridin-3-yl)-5-(2,5-dimethyl-2H-1,2,3-triazol-4-yl)-3H-imidazo[4,5-b]pyridin-3-yl)phenyl)methanol

To a mixture of 3-(3-(4-(((tert-butyldimethylsilyl)oxy)methyl)phenyl)-5-(2,5-dimethyl-2H-1,2,3-triazol-4-yl)-3H-imidazo[4,5-b]pyridin-2-yl)pyridin-2-amine (380 mg, 721 μmol) in THF (4 mL) was added TBAF (1 M in THF, 1.08 mL). The mixture was stirred at 20° C. for 1 hr. The mixture was diluted with H₂O (20 mL) and extracted with EtOAc (50 mL×2). The combined organic layers were washed with brine (20 mL), dried over anhydrous Na₂SO₄, filtered and concentrated to give (4-(2-(2-aminopyridin-3-yl)-5-(2,5-dimethyl-2H-1,2,3-triazol-4-yl)-3H-imidazo[4,5-b]pyridin-3-yl)phenyl)methanol (270 mg, 655 μmol, yield: 91%) as a brown solid. MS: m/z=413.2 [M+H]⁺. ¹H NMR (400 MHz, Dimethylsulfoxide-d₆) δ 8.25 (d, J=8.4 Hz, 1H), 8.01 (dd, J=4.8, 2.0 Hz, 1H), 7.91 (d, J=8.4 Hz, 1H), 7.49-7.42 (m, 4H), 7.24 (dd, J=8.0, 2.0 Hz, 1H), 6.96 (s, 2H), 6.43 (dd, J=7.6, 4.8 Hz, 1H), 5.35 (t, J=6.0 Hz, 1H), 4.59 (d, J=6.0 Hz, 2H), 4.13 (s, 3H), 2.39 (s, 3H).

### Step 4: 3-(3-(4-(Chloromethyl)phenyl)-5-(2,5-dimethyl-2H-1,2,3-triazol-4-yl)-3H-imidazo[4,5-b]pyridin-2-yl)pyridin-2-amine

To a mixture of (4-(2-(2-aminopyridin-3-yl)-5-(2,5-dimethyl-2H-1,2,3-triazol-4-yl)-3H-imidazo[4,5-b]pyridin-3-yl)phenyl)methanol (270 mg, 654 μmol) in CH₂Cl₂ (3 mL) was added SOCl₂ (143 μL, 1.96 mmol). The mixture was stirred at 20° C. for 1 hr. The mixture was concentrated to give 3-(3-(4-(chloromethyl)phenyl)-5-(2,5-dimethyl-2H-1,2,3-triazol-4-yl)-3H-imidazo[4,5-b]pyridin-2-yl)pyridin-2-amine (Intermediate 294, 282 mg) was obtained as a brown solid. MS: m/z=431.2, 433.1 [M+H]⁺.

### Intermediate 295: 3-(3-(4-(Chloromethyl)phenyl)-5-(2-cyclopropyl-2H-1,2,3-triazol-4-yl)-3H-imidazo[4,5-b]pyridin-2-yl)pyridin-2-amine

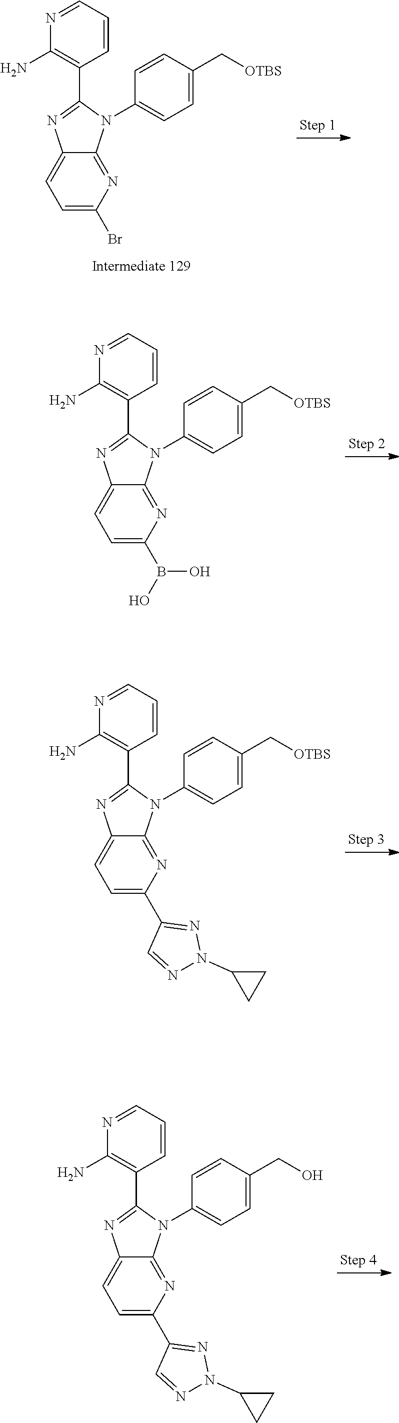 [SMILES: CC(C)(C)[Si](C)(C)OCc1ccc(-n2c(-c3cccnc3N)nc3ccc(Br)nc32)cc1 ; CC(C)(C)[Si](C)(C)OCc1ccc(-n2c(-c3cccnc3N)nc3ccc(B(O)O)nc32)cc1 ; CC(C)(C)[Si](C)(C)OCc1ccc(-n2c(-c3cccnc3N)nc3ccc(-c4cnn(C5CC5)n4)nc32)cc1 ; Nc1ncccc1-c1nc2ccc(-c3cnn(C4CC4)n3)nc2n1-c1ccc(CO)cc1]

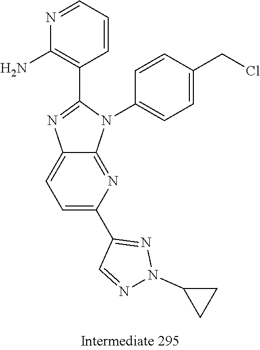 [SMILES: Nc1ncccc1-c1nc2ccc(-c3cnn(C4CC4)n3)nc2n1-c1ccc(CCl)cc1]

### Step 1: (2-(2-Aminopyridin-3-yl)-3-(4-(((tert-butyldimethylsilyl)oxy)methyl)phenyl)-3H-imidazo[4,5-b]pyridin-5-yl)boronic acid

A mixture of Intermediate 129 (500 mg, 980 μmol), 4,4,4′,4′,5,5,5′,5′-octamethyl-2,2′-bi(1,3,2-dioxaborolane) (497 mg, 1.96 mmol), KOAc (289 mg, 2.94 mmol) and Pd(dppf)Cl₂ (72 mg, 98.0 μmol) in 1,4-dioxane (5 mL) was degassed and purged with N₂ three times. The mixture was stirred at 80° C. for 4 hr under N₂ atmosphere. (2-(2-Aminopyridin-3-yl)-3-(4-(((tert-butyldimethylsilyl)oxy)methyl)phenyl)-3H-imidazo[4,5-b]pyridin-5-yl)boronic acid was obtained as a black liquid, which was used in the next step without work-up and purification. MS: m/z=476.1 [M+H]⁺.

### Step 2: 3-(3-(4-(((tert-Butyldimethylsilyl)oxy)methyl)phenyl)-5-(2-cyclopropyl-2H-1,2,3-triazol-4-yl)-3H-imidazo[4,5-b]pyridin-2-yl)pyridin-2-amine

To a solution of (2-(2-aminopyridin-3-yl)-3-(4-(((tert-butyldimethylsilyl)oxy)methyl)phenyl)-3H-imidazo[4,5-h]pyridin-5-yl)boronic acid (466 mg, 979 μmol) and 4-bromo-2-cyclopropyl-triazole (221 mg, 1.18 mmol) in 1,4-dioxane (15 mL) and H₂O (3 mL) were added Pd(dppf)Cl₂ (71.7 mg, 97.9 μmol) and Cs₂CO₃ (957 mg, 2.94 mmol) at 20° C. The mixture was stirred at 80° C. for 2 hr. The mixture was diluted with H₂O (50 mL) and extracted with EtOAc (50 mL×2). The combined organic layers were washed with brine (50 mL), dried over anhydrous Na₂SO₄, filtered and concentrated under reduced pressure. After purified by silica gel flash chromatography (EtOAc in petroleum ether=35%-100/6), 3-(3-(4-(((tert-butyldimethylsilyl)oxy)methyl)phenyl)-5-(2-cyclopropyl-2H-1,2,3-triazol-4-yl)-3H-imidazo[4,5-b]pyridin-2-yl)pyridin-2-amine (440 mg, yield: 83% for two steps) was obtained as a black oil. MS: m/z=539.3 [M+H]⁺. ¹H NMR (400 MHz, Dimethylsulfoxide-d₆) δ 8.27 (d, J=8.4 Hz, 1H), 8.01-7.98 (m, 2H), 7.93 (d, J=8.4 Hz, 1H), 7.50-7.44 (m, 4H), 7.18 (dd, J=7.6, 1.6 Hz, 1H), 6.97 (br s, 2H), 6.38 (dd, J=7.6, 4.8 Hz, 1H), 4.82 (s, 2H), 1.27-1.22 (m, 3H), 1.15-1.10 (m, 2H), 0.92 (s, 9H), 0.11 (s, 6H).

### Step 3: (4-(2-(2-Aminopyridin-3-yl)-5-(2-cyclopropyl-2H-1,2,3-triazol-4-yl)-3H-imidazo[4,5-b]pyridin-3-yl)phenyl)methanol

To a solution of 3-(3-(4-(((tert-butyldimethylsilyl)oxy)methyl)phenyl)-5-(2-cyclopropyl-2H-1,2,3-triazol-4-yl)-3H-imidazo[4,5-b]pyridin-2-yl)pyridin-2-amine (220 mg, 408 μmol) in THF (5 mL) was added TBAF (1 M in THF, 613 μL) at 20° C. The reaction mixture was stirred at 20° C. for 0.5 hr. The mixture was diluted with H₂O (10 mL) and extracted with EtOAc (10 mL×2). The combined organic layers were washed with brine (10 mL), dried over anhydrous Na₂SO₄, filtered and concentrated. (4-(2-(2-Aminopyridin-3-yl)-5-(2-cyclopropyl-2H-1,2,3-triazol-4-yl)-3H-imidazo[4,5-b]pyridin-3-yl)phenyl)methanol (170 mg) was obtained as a gray solid and used in the next step directly. MS: m/z=425.2 [M+H]⁺.

### Step 4: 3-(3-(4-(Chloromethyl)phenyl)-5-(2-cyclopropyl-2H-1,2,3-triazol-4-yl)-3H-imidazo[4,5-b]pyridin-2-yl)pyridin-2-amine

To a solution of (4-(2-(2-aminopyridin-3-yl)-5-(2-cyclopropyl-2H-1,2,3-triazol-4-yl)-3H-imidazo[4,5-b]pyridin-3-yl)phenyl)methanol (170 mg, 401 μmol) in CH₂Cl₂ (5 mL) was added SOCl₂ (143 mg, 1.20 mmol, 87.3 μL). The reaction mixture was stirred at 20° C. for 1 hr and concentrated under reduced pressure. 3-(3-(4-(Chloromethyl)phenyl)-5-(2-cyclopropyl-2H-1,2,3-triazol-4-yl)-3H-imidazo[4,5-b]pyridin-2-yl)pyridin-2-amine (Intermediate 295, 170 mg) was obtained as a gray solid and used in the next step directly. MS: m/z=443.1, 445.2 [M+H]⁺.

### Intermediate 296: 4-Bromo-2-methyl-2H-1,2,3-triazole-5-d

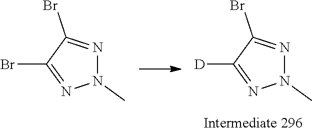

To a solution of 4,5-dibromo-2-methyl-2H-1,2,3-triazole (1 g, 4.15 mmol) in THF (10 mL) was degassed and purged with N₂ three times. n-BuLi (2.5 M, 2.49 mL) was added at −78° C. under N₂ atmosphere. The mixture was stirred at −78° C. for 0.5 hr under N₂ atmosphere. The reaction mixture was quenched with D₂O (166 mg, 8.30 mmol) at −78° C. and stirred at −78° C. for 2 hr under N₂ atmosphere. The reaction mixture was diluted with H₂O (10 mL) and extracted with CH₂Cl₂ 30 mL (15 mL×2). The combined organic layers were washed with brine (30 mL), dried over anhydrous Na₂SO₄, filtered and concentrated under reduced pressure. 4-Bromo-2-methyl-2H-1,2,3-triazole-5-d (Intermediate 296, 200 mg, yield: 30%) was obtained as a light-yellow oil. ¹H NMR (400 MHz, Chloroform-d) δ 4.18 (s, 3H).

### Intermediate 297: 3-(3-(4-(Chloromethyl)phenyl)-5-(2-methyl-2H-1,2,3-triazol-4-yl-5-d)-3H-imidazo[4,5-b]pyridin-2-yl)pyridin-2-amine

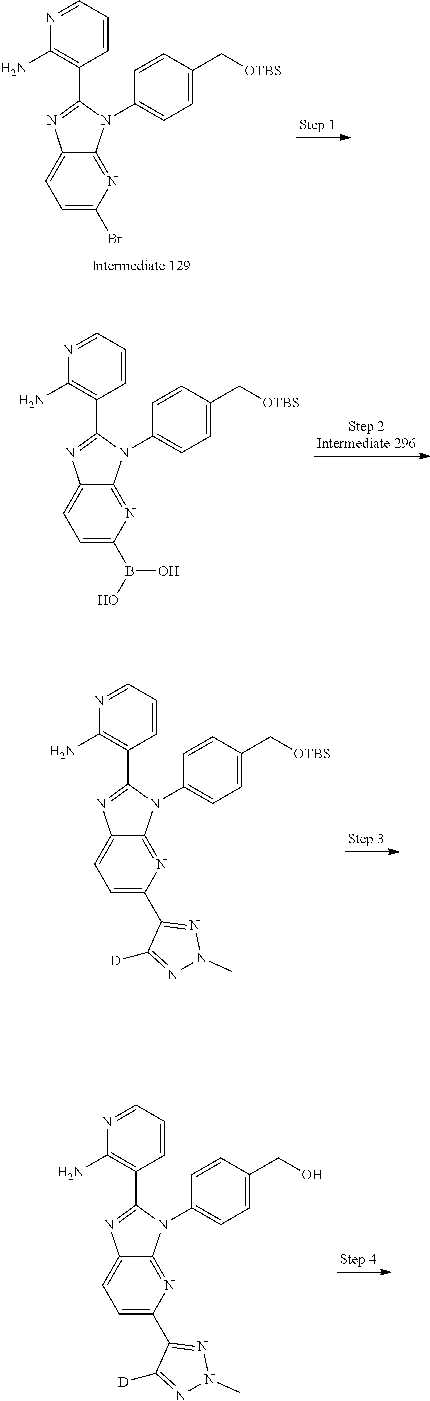 [SMILES: CC(C)(C)[Si](C)(C)OCc1ccc(-n2c(-c3cccnc3N)nc3ccc(Br)nc32)cc1 ; CC(C)(C)[Si](C)(C)OCc1ccc(-n2c(-c3cccnc3N)nc3ccc(B(O)O)nc32)cc1 ; [2H]c1nn(C)nc1-c1ccc2nc(-c3cccnc3N)n(-c3ccc(CO[Si](C)(C)C(C)(C)C)cc3)c2n1 ; [2H]c1nn(C)nc1-c1ccc2nc(-c3cccnc3N)n(-c3ccc(CO)cc3)c2n1]

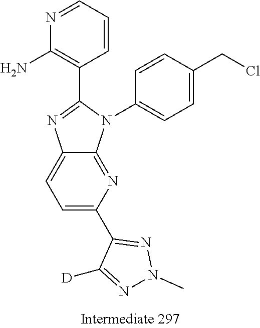 [SMILES: [2H]c1nn(C)nc1-c1ccc2nc(-c3cccnc3N)n(-c3ccc(CCl)cc3)c2n1]

### Step 1: (2-(2-Aminopyridin-3-yl)-3-(4-(((tert-butyldimethylsilyl)oxy)methyl)phenyl)-3H-imidazo[4,5-b]pyridin-5-yl)boronic acid

A mixture of Intermediate 129 (250 mg, 490 μmol), 4,4,4′,4′,5,5,5′,5′-octamethyl-2,2′-bi(1,3,2-dioxaborolane) (187 mg, 735 μmol), KOAc (144 mg, 1.47 mmol) and Pd(dppf)Cl₂ (35.8 mg, 50 μmol) in 1,4-dioxane (3 mL) was degassed and purged with N₂ three times. The mixture was stirred at 90° C. for 2 hr under N₂ atmosphere. (2-(2-Aminopyridin-3-yl)-3-(4-(((tert-butyldimethylsilyl)oxy)methyl)phenyl)-3H-imidazo[4,5-b]pyridin-5-yl)boronic acid was obtained as a black liquid, which was used in the next step without work-up and purification. MS: m/z=476.2 [M+H]⁺.

### Step 2: 3-(3-(4-(((tert-Butyldimethylsilyl)oxy)methyl)phenyl)-5-(2-methyl-2H-1,2,3-triazol-4-yl-5-d)-3H-imidazo[4,5-b]pyridin-2-yl)pyridin-2-amine

A mixture of (2-(2-aminopyridin-3-yl)-3-(4-(((tert-butyldimethylsilyl)oxy)methyl)phenyl)-3H-imidazo[4,5-b]pyridin-5-yl)boronic acid (200 mg, 421 μmol), Intermediate 296 (68.6 mg, 421 μmol), Cs₂CO₃ (411 mg, 1.26 mmol) and Pd(dppf)Cl₂ (30.8 mg, 42.1 μmol) in 1,4-dioxane (5 mL) and H₂O (1 mL) was degassed and purged with N₂ three 3 times. The mixture was stirred at 80° C. for 16 hr under N₂ atmosphere. The reaction mixture was quenched with H₂O (20 mL) and extracted with CH₂Cl₂ (15 mL×2). The combined organic layers were washed with brine (30 mL), dried over Na₂SO₄, filtered and concentrated under reduced pressure. After purified by silica gel flash chromatography (Eluent of 10%˜40% EtOAc in petroleum ether), 3-(3-(4-(((tert-butyldimethylsilyl)oxy)methyl)phenyl)-5-(2-methyl-2H-1,2,3-triazol-4-yl-5-d)-3H-imidazo[4,5-b]pyridin-2-yl)pyridin-2-amine (130 mg, yield: 39% for two steps) was obtained as a brown solid. MS: m/z=514.1 [M+H]⁺. ¹H NMR (400 MHz, Chloroform-d) 68.12 (d, J=8.4 Hz, 1H), 8.07-8.03 (m, 1H), 7.38 (d, J=8.0 Hz, 2H), 7.31 (d, J=8.4 Hz, 2H), 7.13 (d, J=7.6 Hz, 1H), 7.02 (d, J=8.0 Hz, 1H), 6.72 (s, 2H), 6.32 (dd, J=8.0, 5.2 Hz, 1H), 4.86 (s, 2H), 4.25 (s, 3H), 0.98 (s, 9H), 0.15 (s, 6H).

### Step 3. (4-(2-(2-Aminopyridin-3-yl)-5-(2-methyl-2H-1,2,3-triazol-4-yl-5-d)-3H-imidazo[4,5-b]pyridin-3-yl)phenyl)methanol

To a solution of 3-(3-(4-(((tert-butyldimethylsilyl)oxy)methyl)phenyl)-5-(2-methyl-2H-1,2,3-triazol-4-yl-5-d)-3H-imidazo[4,5-b]pyridin-2-yl)pyridin-2-amine (130 mg, 253 μmol) in THF (2 mL) was added TBAF (506 μL, 1 M in THF). The mixture was stirred at 25° C. for 1 hr. The reaction mixture was quenched with H₂O (20 mL) at 25° C. and extracted with EtOAc (15 mL×2). The combined organic layers were washed with brine (50 mL×3), dried over Na₂SO₄, filtered and concentrated under reduced pressure. After triturated with CH₂Cl₂ (10 mL) at 25° C. for 10 min, the liquid was collected and concentrated under reduced pressure. (4-(2-(2-Aminopyridin-3-yl)-5-(2-methyl-2H-1,2,3-triazol-4-yl-5-d)-3H-imidazo[4,5-b]pyridin-3-yl)phenyl)methanol (80 mg, yield: 47%) was obtained as a brown solid. MS: m/z=400.2 [M+H]⁺.

### Step 4: 3-(3-(4-(Chloromethyl)phenyl)-5-(2-methyl-2H-1,2,3-triazol-4-yl-5-d)-3H-imidazo[4,5-b]pyridin-2-yl)pyridin-2-amine

To a solution of (4-(2-(2-aminopyridin-3-yl)-5-(2-methyl-2H-1,2,3-triazol-4-yl-5-d)-3H-imidazo[4,5-b]pyridin-3-yl)phenyl)methanol (80 mg, 200 μmol) in CH₂Cl₂ (3 mL) was added SOCl₂ (71.5 mg, 600 μmol). The mixture was stirred at 40° C. for 0.2 hr. The reaction was concentrated under reduced pressure. And the solid was diluted with NaHCO₃ (10 mL) and extracted with CH₂Cl₂ (15 mL×2). The combined organic layers were washed with brine (30 mL), dried over anhydrous Na₂SO₄, filtered and concentrated under reduced pressure. After purified by silica gel flash chromatography (Eluent of 0%˜4% MeOH in CH₂Cl₂), 3-(3-(4-(chloromethyl)phenyl)-5-(2-methyl-2H-1,2,3-triazol-4-yl-5-d)-3H-imidazo[4,5-b]pyridin-2-yl)pyridin-2-amine (Intermediate 297, 23 mg, yield: 27%) was obtained as a yellow solid. MS: m/z=418.1 [M+H]⁺.

### Intermediate 298: 4-(2-(3-Aminopyrazin-2-yl)-5-bromo-3H-imidazo[4,5-b]pyridin-3-yl)benzyl acetate

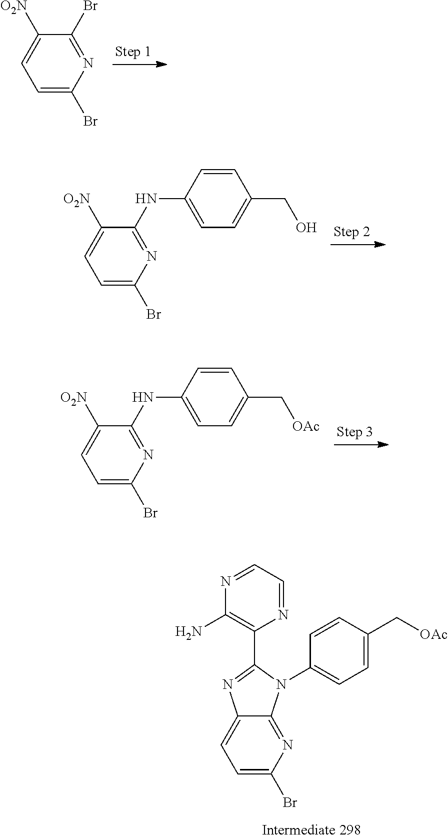 [SMILES: O=[N+]([O-])c1ccc(Br)nc1Br ; O=[N+]([O-])c1ccc(Br)nc1Nc1ccc(CO)cc1 ; CC(=O)OCc1ccc(Nc2nc(Br)ccc2[N+](=O)[O-])cc1 ; CC(=O)OCc1ccc(-n2c(-c3nccnc3N)nc3ccc(Br)nc32)cc1]

### Step 1: (4-((6-Bromo-3-nitropyridin-2-yl)amino)phenyl)methanol

A mixture of 2,6-dibromo-3-nitropyridine (5 g, 17.7 mmol), (4-aminophenyl)methanol (2.18 g, 17.7 mmol), DIEA (6.88 g, 53.2 mmol) in 1,4-dioxane (50 mL) was degassed and purged with N₂ three times and stirred at 45° C. for 2 hr under N₂ atmosphere. The reaction mixture was quenched with H₂O (100 mL) at 25° C. and extracted with CH₂Cl₂ (100 mL×2). The combined organic layers were washed with brine (100 mL), dried over anhydrous Na₂SO₄, filtered and concentrated under reduced pressure. (4-((6-Bromo-3-nitropyridin-2-yl)amino)phenyl)methanol (5.75 g) was obtained as a yellow solid. MS: m/z=324.1, 326.1 [M+H]⁺. ¹H NMR (400 MHz, Chloroform-d) δ 10.24 (br s, 1H), 8.33 (d, J=8.8 Hz, 1H), 7.71-7.58 (m, 2H), 7.43-7.40 (m, 2H), 6.97 (d, J=8.4 Hz, 1H), 4.71 (br s, 2H).

### Step 2: 4-((6-Bromo-3-nitropyridin-2-yl)amino)benzyl acetate

To a solution of (4-((6-bromo-3-nitropyridin-2-yl)amino)phenyl)methanol (5.75 g, 17.7 mmol) in CH₂Cl₂ (60 mL) were added DMAP (217 mg, 1.77 mmol), TEA (5.39 g, 53.2 mmol) and Ac₂O (2.72 g, 26.6 mmol). The mixture was stirred at 25° C. for 2 hr. The reaction mixture was quenched with H₂O (100 mL) at 25° C. and extracted with CH₂Cl₂ (100 mL×2). The combined organic layers were washed with brine (100 mL), dried over anhydrous Na₂SO₄, filtered, and concentrated under reduced pressure. After purified by silica gel flash chromatography (Eluent of 0%˜15% EtOAc in petroleum ether), 4-((6-bromo-3-nitropyridin-2-yl)amino)benzyl acetate (4.9 g, yield: 75% for two steps) was obtained as a yellow solid. ¹H NMR (400 MHz, Chloroform-d) δ 10.25 (br s, 1H), 8.33 (d, J=8.4 Hz, 1H), 7.66 (d, J=8.4 Hz, 2H), 7.40 (d, J=8.4 Hz, 2H), 6.98 (d, J=8.4 Hz, 1H), 5.11 (s, 2H), 2.11 (s, 3H).

### Step 3: 4-(2-(3-Aminopyrazin-2-yl)-5-bromo-3H-imidazo[4,5-b]pyridin-3-yl)benzyl acetate

To a solution of 4-((6-bromo-3-nitropyridin-2-yl)amino)benzyl acetate (1 g, 2.73 mmol) in DMSO (10 mL) were added Na₂S₂O₄ (2.16 g, 10.9 mmol, 88% purity) and 3-aminopyrazine-2-carbaldehyde (403 mg, 3.28 mmol). The mixture was stirred at 100° C. for 3 hr. The reaction mixture was quenched with H₂O (50 mL) at 25° C. and extracted with CH₂Cl₂ (50 mL×2). The combined organic layers were washed with brine (50 mL×3), dried over anhydrous Na₂SO₄, filtered and concentrated under reduced pressure. After purified by silica gel flash chromatography (Eluent of 0%˜22% EtOAc in petroleum ether), 4-(2-(3-aminopyrazin-2-yl)-5-bromo-3H-imidazo[4,5-b]pyridin-3-yl)benzyl acetate (Intermediate 298, 270 mg, yield: 21%) was obtained as a yellow solid. MS: m/z=439.1, 441.1 [M+H]⁺. ¹H NMR (400 MHz, Chloroform-d) δ 7.99-7.93 (m, 2H), 7.56 (d, J=2.4 Hz, 1H), 7.50-7.44 (m, 3H), 7.33-7.29 (m, 2H), 7.17-6.85 (m, 2H), 5.21 (s, 2H), 2.17 (s, 3H).

### Intermediate 299: (1-(Difluoromethyl)-6-oxo-1,6-dihydropyridin-3-yl)boronic acid

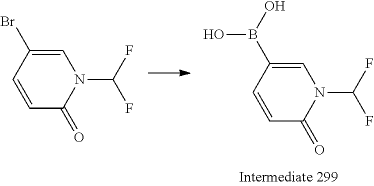 [SMILES: O=c1ccc(Br)cn1C(F)F ; O=c1ccc(B(O)O)cn1C(F)F]

A mixture of 5-bromo-1-(difluoromethyl)pyridin-2(1H)-one (50 mg, 223 μmol), 4,4,4′,4′,5,5,5′,5′-octamethyl-2,2′-bi(1,3,2-dioxaborolane) (113 mg, 446 μmol), Pd(dppf)Cl₂ (16.3 mg, 22.3 μmol) and KOAc (65.7 mg, 670 μmol) in 1,4-dioxane (2 mL) was degassed and purged with N₂ three times. The mixture was stirred at 80° C. for 2 hr under N₂ atmosphere. (1-(Difluoromethyl)-6-oxo-1,6-dihydropyridin-3-yl)boronic acid (Intermediate 299) was obtained as a black liquid, the mixture was used for next step directly and without work-up and purification. MS: m/z=189.7 [M+H]⁺.

### Intermediate 300 & 301: 4-Bromo-2-(fluoromethyl)-2H-1,2,3-triazole & 4-Bromo-1-(fluoromethyl)-1H-1,2,3-triazole

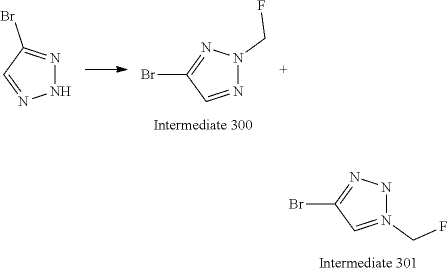

A mixture of 4-bromo-2H-1,2,3-triazole (3 g, 20.3 mmol), bromofluoromethane (4.58 g, 40.6 mmol), DIEA (7.86 g, 60.8 mmol) in DMF (50 mL) was degassed and purged with N₂ three times. The mixture was stirred at 80° C. for 1 hr under N₂ atmosphere. The reaction mixture was diluted with H₂O (100 mL) and extracted with CH₂Cl₂ (50 mL×3). The combined organic layers were washed with brine (50 mL×3), dried over Na₂SO₄, filtered and concentrated under reduced pressure. The mixture (3.2 g, crude) of 4-bromo-2-(fluoromethyl)-2H-1,2,3-triazole (Intermediate 300) and 4-bromo-1-(fluoromethyl)-1H-1,2,3-triazole (Intermediate 301) was obtained as a brown solid. ¹H NMR (400 MHz, Chloroform-d) δ 7.73 (s, 1H), 6.27-6.12 (m, 2H). ¹⁹F NMR (400 MHz, Chloroform-d) δ −166.057. ¹H NMR (400 MHz, Chloroform-d) 7.87 (s, 1H), 6.35-6.20 (m, 2H). ¹⁹F NMR (400 MHz, Chloroform-d) 6-167.648.

### Intermediate 302 & 303: 3-(3-(4-(((tert-Butyldimethylsilyl)oxy)methyl)phenyl)-5-(2-(fluoromethyl)-2H-1,2,3-triazol-4-yl)-3H-imidazo[4,5-b]pyridin-2-yl)pyridin-2-amine & 3-(3-(4-(((tert-Butyldimethylsilyl)oxy)methyl)phenyl)-5-(1-(fluoromethyl)-1H-1,2,3-triazol-4-yl)-3H-imidazo[4,5-b]pyridin-2-yl)pyridin-2-amine

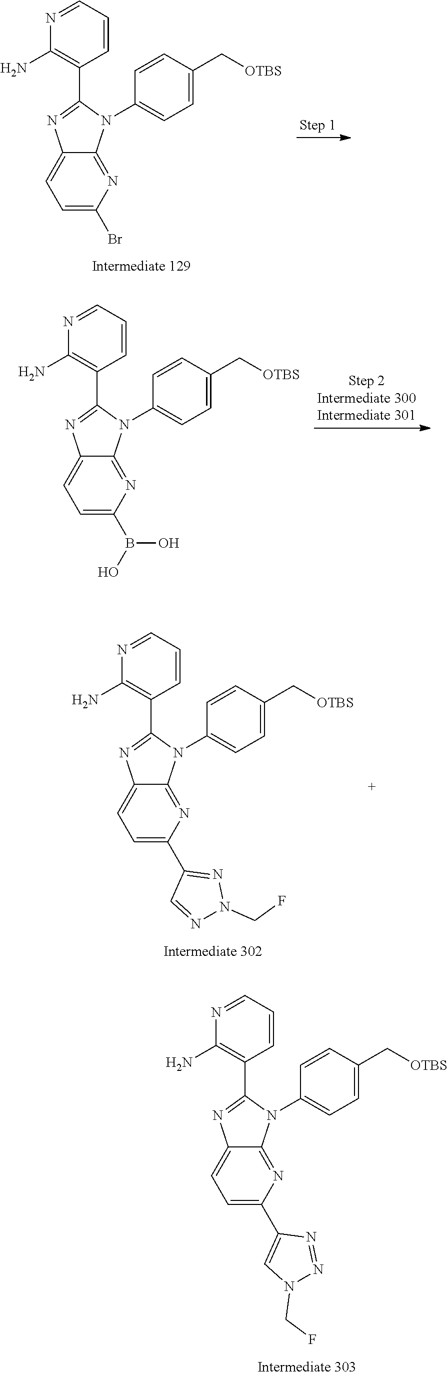 [SMILES: CC(C)(C)[Si](C)(C)OCc1ccc(-n2c(-c3cccnc3N)nc3ccc(Br)nc32)cc1 ; CC(C)(C)[Si](C)(C)OCc1ccc(-n2c(-c3cccnc3N)nc3ccc(B(O)O)nc32)cc1 ; CC(C)(C)[Si](C)(C)OCc1ccc(-n2c(-c3cccnc3N)nc3ccc(-c4cnn(CF)n4)nc32)cc1 ; CC(C)(C)[Si](C)(C)OCc1ccc(-n2c(-c3cccnc3N)nc3ccc(-c4cn(CF)nn4)nc32)cc1]

### Step 1: (2-(2-Aminopyridin-3-yl)-3-(4-(((tert-butyldimethylsilyl)oxy)methyl)phenyl)-3H-imidazo[4,5-b]pyridin-5-yl)boronic acid

A mixture of Intermediate 129 (5 g, 9.79 mmol), 4,4,4′,4′,5,5,5′,5′-octamethyl-2,2′-bi(1,3,2-dioxaborolane) (4.97 g, 19.6 mmol), KOAc (2.88 g, 29.4 mmol) and Pd(dppf)Cl₂ (717 mg, 979 μmol) in 1,4-dioxane (60 mL) was degassed and purged with N₂ three times. The mixture was stirred at 90° C. for 2 hr under N₂ atmosphere. (2-(2-Aminopyridin-3-yl)-3-(4-(((tert-butyldimethylsilyl)oxy)methyl)phenyl)-3H-imidazo[4,5-b]pyridin-5-yl)boronic acid was obtained as a black liquid, which was used in the next step without work-up and purification. MS: m/z=476.4 [M+H]⁺.

### Step 2: 3-(3-(4-(((tert-Butyldimethylsilyl)oxy)methyl)phenyl)-5-(2-(fluoromethyl)-2H-1,2,3-triazol-4-yl)-3H-imidazo[4,5-b]pyridin-2-yl)pyridin-2-amine & 3-(3-(4-(((tert-butyldimethylsilyl)oxy)methyl)phenyl)-5-(1-(fluoromethyl)-1H-1,2,3-triazol-4-yl)-3H-imidazo[4,5-b]pyridin-2-yl)pyridin-2-amine

A mixture of (2-(2-aminopyridin-3-yl)-3-(4-(((tert-butyldimethylsilyl)oxy)methyl)phenyl)-3H-imidazo[4,5-b]pyridin-5-yl)boronic acid (4 g, 8.41 mmol), Intermediate 300 (1.51 g, 8.41 mmol, contained Intermediate 301), Pd(dppf)Cl₂ (616 mg, 841 μmol), Cs₂CO₃ (8.22 g, 25.2 mmol) in 1,4-dioxane (40 mL) and H₂O (10 mL) was degassed and purged with N₂ three times. The mixture was stirred at 100° C. for 4 hr under N₂ atmosphere. The residue was diluted with H₂O (50 mL) and extracted with CH₂Cl₂ (50 mL×3). The combined organic layers were washed with brine (20 mL×2), dried over Na₂SO₄, filtered and concentrated under reduced pressure. After purified by prep-HPLC (column: Phenomenex luna C18 (250×70 mm, 10 μm); mobile phase: [water (NH₄HCO₃)-ACN]; gradient: 60%-90% B over 5 min), 3-(3-(4-(((tert-butyldimethylsilyl)oxy)methyl)phenyl)-5-(2-(fluoromethyl)-2H-1,2,3-triazol-4-yl)-3H-imidazo[4,5-b]pyridin-2-yl)pyridin-2-amine as a yellow solid (Intermediate 302, 2.2 g, yield: 48% for two steps) was obtained as a yellow solid. MS: m/z=531.2 [M+H]⁺. ¹H NMR (400 MHz, Chloroform-d) δ 8.21 (s, 1H), 8.17-8.13 (m, 1H), 8.09-8.04 (m, 2H), 7.51 (d, J=8.0 Hz, 2H), 7.39 (d, J=8.4 Hz, 2H), 7.13 (dd, J=7.6, 1.6 Hz, 1H), 6.67 (br s, 2H), 6.38 (dd, J=8.0, 5.2 Hz, 1H), 6.28 (d, J=52.0 Hz, 2H), 4.87 (s, 2H), 0.98 (s, 9H), 0.16 (s, 6H). ¹⁹F NMR (400 MHz, Chloroform-d) δ −166.057. 3-(3-(4-(((tert-butyldimethylsilyl)oxy)methyl)phenyl)-5-(1-(fluoromethyl)-1H-1,2,3-triazol-4-yl)-3H-imidazo[4,5-b]pyridin-2-yl)pyridin-2-amine (Intermediate 303, 550 mg, yield. 8.9% for two steps) was obtained as a brown solid. MS: m/z=531.1 [M+H]⁺. ¹H NMR (400 MHz, Chloroform-d) δ 8.30-8.23 (m, 2H), 8.18-8.14 (m, 1H), 8.09-8.06 (m, 1H), 7.50 (d, J=8.4 Hz, 2H), 7.37 (d, 1=8.4 Hz, 2H), 7.11 (dd, J=8.0, 1.6 Hz, 1H), 6.67 (br s, 2H), 6.39-6.35 (m, 2H), 6.24-6.22 (m, 1H), 4.87 (s, 2H), 0.99 (s, 9H), 0.17 (s, 6H). ¹⁹F NMR (400 MHz, Chloroform) 6-167.296.

### Intermediate 304: 3-(3-(4-(Chloromethyl)phenyl)-5-(2-(fluoromethyl)-2H-1,2,3-triazol-4-yl)-3H-imidazo[4,5-b]pyridin-2-yl)pyridin-2-amine

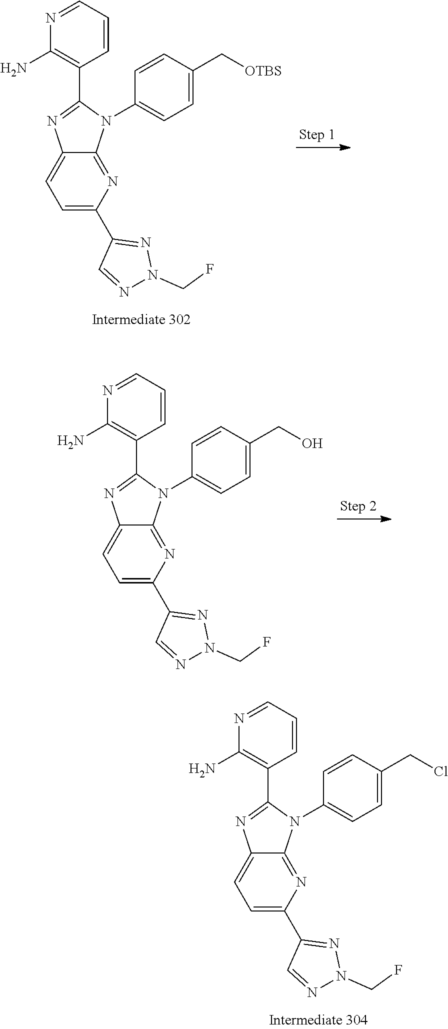 [SMILES: CC(C)(C)[Si](C)(C)OCc1ccc(-n2c(-c3cccnc3N)nc3ccc(-c4cnn(CF)n4)nc32)cc1 ; Nc1ncccc1-c1nc2ccc(-c3cnn(CF)n3)nc2n1-c1ccc(CO)cc1 ; Nc1ncccc1-c1nc2ccc(-c3cnn(CF)n3)nc2n1-c1ccc(CCl)cc1]

### Step 1: (4-(2-(2-Aminopyridin-3-yl)-5-(2-(fluoromethyl)-2H-1,2,3-triazol-4-yl)-3H-imidazo[4,5-b]pyridin-3-yl)phenyl)methanol

To a solution of Intermediate 302 (2.2 g, 4.15 mmol) in THF (20 mL) was added TBAF (6.22 mL, 1 M in THF). The residue was diluted with H₂O (50 mL) and extracted with CH₂Cl₂ (50 mL×3). The combined organic layers were washed with brine (20 mL×2), dried over Na₂SO₄, filtered and concentrated under reduced pressure. (4-(2-(2-Aminopyridin-3-yl)-5-(2-(fluoromethyl)-2H-1,2,3-triazol-4-yl)-3H-imidazo[4,5-b]pyridin-3-yl)phenyl)methanol (1.5 g, crude) was obtained as a brown solid which was used into the next step directly. MS: m/z=417.3 [M+H]⁺. ¹H NMR (400 MHz, Dimethylsulfoxide-d₆) δ 8.38-8.27 (m, 2H), 8.07-7.95 (m, 2H), 7.51-7.42 (m, 4H), 7.23 (dd, J=7.6, 1.6 Hz, 1H), 6.91 (br s, 2H), 6.55-6.53 (m, 1H), 6.44-6.40 (m, 2H), 5.37 (br t, J=5.6 Hz, 1H), 4.60 (d, J=5.6 Hz, 2H). ¹⁹F NMR (400 MHz, Dimethylsulfoxide-d₆) δ −166.272.

### Step 2: 3-(3-(4-(Chloromethyl)phenyl)-5-(2-(fluoromethyl)-2H-1,2,3-triazol-4-yl)-3H-imidazo[4,5-b]pyridin-2-yl)pyridin-2-amine

To a solution of (4-(2-(2-aminopyridin-3-yl)-5-(2-(fluoromethyl)-2H-1,2,3-triazol-4-yl)-3H-imidazo[4,5-b]pyridin-3-yl)phenyl)methanol (1.5 g, 3.60 mmol) in CH₂Cl₂ (5 mL) was added SOCl₂ (857 mg, 7.20 mmol). The reaction mixture was stirred at 40° C. for 1 hr. The reaction mixture was concentrated. 3-(3-(4-(Chloromethyl)phenyl)-5-(2-(fluoromethyl)-2H-1,2,3-triazol-4-yl)-3H-imidazo[4,5-b]pyridin-2-yl)pyridin-2-amine (Intermediate 304, 1.6 g, crude, HCl salt) was obtained as a yellow solid, which was used into the next step directly. MS: m/z=435.0, 437.0 [M+H]⁺.

### Intermediate 305: 3-(3-(4-(Chloromethyl)phenyl)-5-(1-(fluoromethyl)-1H-1,2,3-triazol-4-yl)-3H-imidazo[4,5-b]pyridin-2-yl)pyridin-2-amine

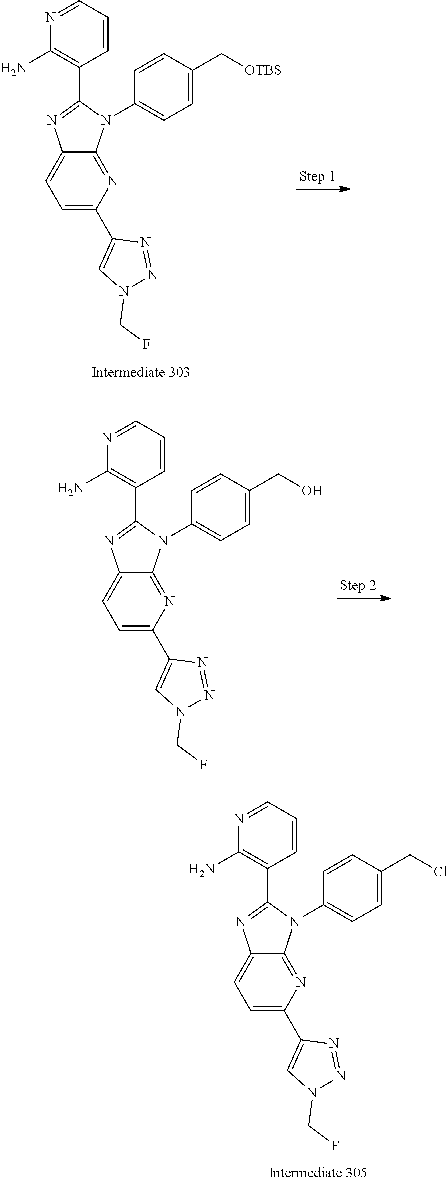 [SMILES: CC(C)(C)[Si](C)(C)OCc1ccc(-n2c(-c3cccnc3N)nc3ccc(-c4cn(CF)nn4)nc32)cc1 ; Nc1ncccc1-c1nc2ccc(-c3cn(CF)nn3)nc2n1-c1ccc(CO)cc1 ; Nc1ncccc1-c1nc2ccc(-c3cn(CF)nn3)nc2n1-c1ccc(CCl)cc1]

### Step 1: (4-(2-(2-Aminopyridin-3-yl)-5-(1-(fluoromethyl)-1H-1,2,3-triazol-4-yl)-3H-imidazo[4,5-b]pyridin-3-yl)phenyl)methanol

To a solution of Intermediate 303 (550 mg, 1.04 mmol) in THF (20 mL) was added TBAF (1.55 mL, 1 M in THF). The residue was diluted with H₂O (10 mL) and extracted with CH₂Cl₂ (20 mL×3). The combined organic layers were washed with brine (20 mL×2), dried over Na₂SO₄, filtered and concentrated under reduced pressure. (4-(2-(2-Aminopyridin-3-yl)-5-(1-(fluoromethyl)-1H-1,2,3-triazol-4-yl)-3H-imidazo[4,5-b]pyridin-3-yl)phenyl)methanol (420 mg, crude) was obtained as a yellow solid which was used into the next step directly. MS: m/z=417.1 [M+H]⁺. ¹H NMR (400 MHz, Dimethylsulfoxide-d₆) δ 8.77 (s, 1H), 8.31 (d, J=8.0 Hz, 1H), 8.12 (d, J=8.4 Hz, 1H), 8.01-7.98 (m, 1H), 7.51-7.40 (m, 4H), 7.22 (dd, J=7.6, 1.6 Hz, 1H), 6.92 (br s, 2H), 6.54 (br s, 1H), 6.44-6.38 (m, 2H), 5.42-5.37 (m, 1H), 4.60 (d, J=5.6 Hz, 2H). ¹⁹F NMR (400 MHz, Dimethylsulfoxide-d₆) δ −166.677.

### Step 2: 3-(3-(4-(Chloromethyl)phenyl)-5-(1-(fluoromethyl)-1H-1,2,3-triazol-4-yl)-3H-imidazo[4,5-b]pyridin-2-yl)pyridin-2-amine

To a solution of (4-(2-(2-aminopyridin-3-yl)-5-(1-(fluoromethyl)-1H-1,2,3-triazol-4-yl)-3H-imidazo[4,5-b]pyridin-3-yl)phenyl)methanol (420 mg, 1.01 mmol) in CH₂Cl₂ (5 mL) was added SOCl₂ (120 mg, 1.01 mmol). The reaction mixture was stirred at 40° C. for 1 hr. The reaction mixture was concentrated. 3-(3-(4-(Chloromethyl)phenyl)-5-(1-(fluoromethyl)-1H-1,2,3-triazol-4-yl)-3H-imidazo[4,5-b]pyridin-2-yl)pyridin-2-amine (Intermediate 305, 450 mg, crude, HCl salt) was obtained as a yellow solid, which was used into the next step directly. MS: m/z=435.1, 437.1 [M+H]⁺.

### Intermediate 306: 4-Bromo-2-(difluoromethyl)-2H-1,2,3-triazole

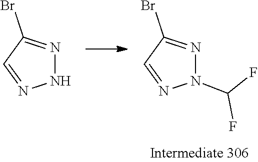 [SMILES: Brc1cn[nH]n1 ; FC(F)n1ncc(Br)n1]

To a solution of 4-bromo-2H-1,2,3-triazole (1 g, 6.76 mmol) and Cs₂CO₃ (6.6 g, 20.3 mmol) in DMSO (30 mL) was added sodium 2-chloro-2,2-difluoroacetate (2.06 g, 13.5 mmol) at 25° C. The mixture was stirred at 90° C. for 16 hr. The mixture was quenched with H₂O (30 mL) and extracted with EtOAc (30 mL×3). The combined organic layers were washed with brine (30 mL×5), dried over Na₂SO₄, filtered and concentrated. After purified by silica gel flash chromatography (Eluent of 0%˜5% EtOAc in petroleum ether), 4-bromo-2-(difluoromethyl)-2H-1,2,3-triazole (Intermediate 306, 150 mg, yield: 11%) was obtained as a yellow oil. ¹H NMR (400 MHz, Dimethylsulfoxide-d₆) δ 9.11 (s, 1H), 8.43-8.09 (m, 1H). ¹⁹F NMR (400 MHz, Dimethylsulfoxide-d₆) δ −96.77.

### Intermediate 307: 3-(3-(4-(Chloromethyl)phenyl)-5-(2-(difluoromethyl)-2H-1,2,3-triazol-4-yl)-3H-imidazo[4,5-b]pyridin-2-yl)pyridin-2-amine

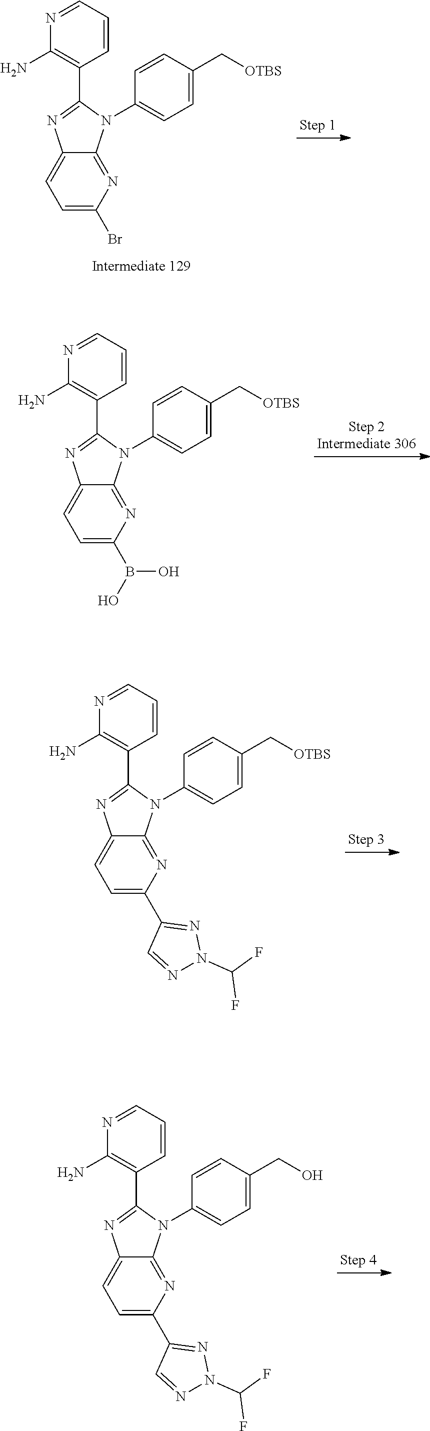 [SMILES: CC(C)(C)[Si](C)(C)OCc1ccc(-n2c(-c3cccnc3N)nc3ccc(Br)nc32)cc1 ; CC(C)(C)[Si](C)(C)OCc1ccc(-n2c(-c3cccnc3N)nc3ccc(B(O)O)nc32)cc1 ; CC(C)(C)[Si](C)(C)OCc1ccc(-n2c(-c3cccnc3N)nc3ccc(-c4cnn(C(F)F)n4)nc32)cc1 ; Nc1ncccc1-c1nc2ccc(-c3cnn(C(F)F)n3)nc2n1-c1ccc(CO)cc1]

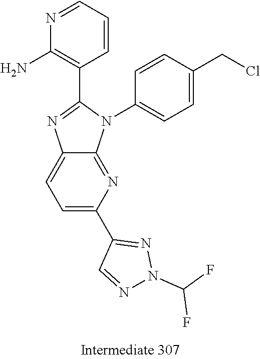 [SMILES: Nc1ncccc1-c1nc2ccc(-c3cnn(C(F)F)n3)nc2n1-c1ccc(CCl)cc1]

### Step 1 (2-(2-Aminopyridin-3-yl)-3-(4-(((ert-butyldimethylsilyl)oxy)methyl)phenyl)-3H-imidazo[4,5-b]pyridin-5-yl)boronic acid

A mixture of Intermediate 129 (1 g, 1.96 mmol), 4,4,4′,4′,5,5,5′,5′-octamethyl-2,2′-bi(1,3,2-dioxaborolane) (995 mg, 3.92 mmol), KOAc (577 mg, 5.88 mmol) and Pd(dppf)Cl₂ (143 mg, 196 μmol) in 1,4-dioxane (10 mL) was degassed and purged with N₂ three times. The mixture was stirred at 90° C. for 8 hr under N₂ atmosphere. (2-(2-Aminopyridin-3-yl)-3-(4-(((tert-butyldimethylsilyl)oxy)methyl)phenyl)-3H-imidazo[4,5-b]pyridin-5-yl)boronic acid was obtained as a black liquid, which was used in the next step without work-up and purification. MS: m/z=476.2 [M+H]⁺.

### Step 2: 3-(3-(4-(((Tert-butyldimethylsilyl)oxy)methyl)phenyl)-5-(2-(difluoromethyl)-2H-1,2,3-triazol-4-yl)-3H-imidazo[4,5-b]pyridin-2-yl)pyridin-2-amine

A mixture of (2-(2-aminopyridin-3-yl)-3-(4-(((tert-butyldimethylsilyl)oxy)methyl)phenyl)-3H-imidazo[4,5-b]pyridin-5-yl)boronic acid (931 mg, 1.96 mmol), Intermediate 306 (388 mg, 1.96 mmol), Pd(dppf)Cl₂ (143 mg, 196 μmol) and Cs₂CO₃ (1.91 mg, 5.87 mmol) in 1,4-dioxane (10 mL) and H₂O (2 mL) was degassed and purged with N₂ three times. The mixture was stirred at 90° C. for 16 hr under N₂ atmosphere. The reaction mixture was quenched with H₂O (10 mL) and extracted with EtOAc (30 mL×3). The combined organic layers were washed with brine (90 mL), dried over Na₂SO₄, filtered and concentrated under reduced pressure. After purified by prep-HPLC (column: Daisogel SP ODS RPS 150×25 mm×5 μm; mobile phase: [water (NH₄HCO₃)-ACN]; gradient: 60%-90% B over 11 min), 3-(3-(4-(((tert-butyldimethylsilyl)oxy)methyl)phenyl)-5-(2-(difluoromethyl)-2H-1,2,3-triazol-4-yl)-3H-imidazo[4,5-b]pyridin-2-yl)pyridin-2-amine (350 mg, yield: 33% for two steps) was obtained as a yellow solid. MS: m/z=549.2 [M+H]⁺. ¹H NMR (400 MHz, Dimethylsulfoxide-d₆) δ 8.93 (s, 1H), 8.33 (d, J=8.4 Hz, 1H), 8.24-8.02 (m, 2H), 7.99 (dd, J=4.8, 1.6 Hz, 1H), 7.53-7.40 (m, 4H), 7.19 (dd, J=7.6, 1.6 Hz, 1H), 6.93 (s, 2H), 6.38 (dd, J=7.6, 4.8 Hz, 1H), 4.80 (s, 2H), 0.92 (s, 9H), 0.10 (s, 6H). ¹⁹F NMR (400 MHz, Dimethylsulfoxide-d₆) δ −96.38.

### Step 3: (4-(2-(2-Aminopyridin-3-yl)-5-(2-(difluoromethyl)-2H-1,2,3-triazol-4-yl)-3H-imidazo[4,5-b]pyridin-3-yl)phenyl)methanol

To a solution of 3-(3-(4-(((tert-butyldimethylsilyl)oxy)methyl)phenyl)-5-(2-(difluoromethyl)-2H-1,2,3-triazol-4-yl)-3H-imidazo[4,5-b]pyridin-2-yl)pyridin-2-amine (320 mg, 583 μmol) in THF (5 mL) was added TBAF (0.5 mL, 1 M in THF). The mixture was stirred at 25° C. for 1 hr. The reaction mixture was quenched with H₂O (5 mL) at 25° C. and extracted with EtOAc (10 mL×3). The combined organic layers were washed with brine (50 mL×3), dried over Na₂SO₄, filtered and concentrated under reduced pressure. (4-(2-(2-Aminopyridin-3-yl)-5-(2-(difluoromethyl)-2H-1,2,3-triazol-4-yl)-3H-imidazo[4,5-b]pyridin-3-yl)phenyl)methanol (300 mg) was obtained as a yellow solid. MS: m/z=435.1 [M+H]⁺. ¹H NMR (400 MHz, Dimethylsulfoxide-d₆) δ 8.96 (s, 1H), 8.40-8.27 (m, 1H), 8.26-8.05 (m, 2H), 8.00 (dd, J=4.8, 1.6 Hz, 1H), 7.52-7.39 (m, 4H), 7.22 (dd, J=7.6, 1.6 Hz, 1H), 6.93 (br s, 2H), 6.42 (dd, J=7.6, 4.8 Hz, 1H), 5.45-5.32 (m, 1H), 4.60 (d, J=5.6 Hz, 2H). ¹⁹F NMR (400 MHz, Dimethylsulfoxide-d₆) δ-96.39.

### Step 4: 3-(3-(4-(Chloromethyl)phenyl)-5-(2-(difluoromethyl)-2H-1,2,3-triazol-4-yl)-3H-imidazo[4,5-b]pyridin-2-yl)pyridin-2-amine

To a solution of (4-(2-(2-aminopyridin-3-yl)-5-(2-(difluoromethyl)-2H-1,2,3-triazol-4-yl)-3H-imidazo[4,5-b]pyridin-3-yl)phenyl)methanol (300 mg, 691 μmol) in CH₂Cl₂ (5 mL) was added SOCl₂ (246 mg, 2.07 mmol). The mixture was stirred at 40° C. for 0.5 hr. The reaction was concentrated under reduced pressure to give 3-(3-(4-(chloromethyl)phenyl)-5-(2-(difluoromethyl)-2H-1,2,3-triazol-4-yl)-3H-imidazo[4,5-b]pyridin-2-yl)pyridin-2-amine (Intermediate 307, 312 mg, HCl salt) was obtained as a yellow solid. MS: m/z=453.1, 455.1 [M+H]⁺.

### Intermediate 308: 3-(3-(4-(Chloromethyl)phenyl)-5-(2-(methyl-d₃)-2H-1,2,3-triazol-4-yl)-3H-imidazo[4,5-b]pyridin-2-yl)pyrazin-2-amine

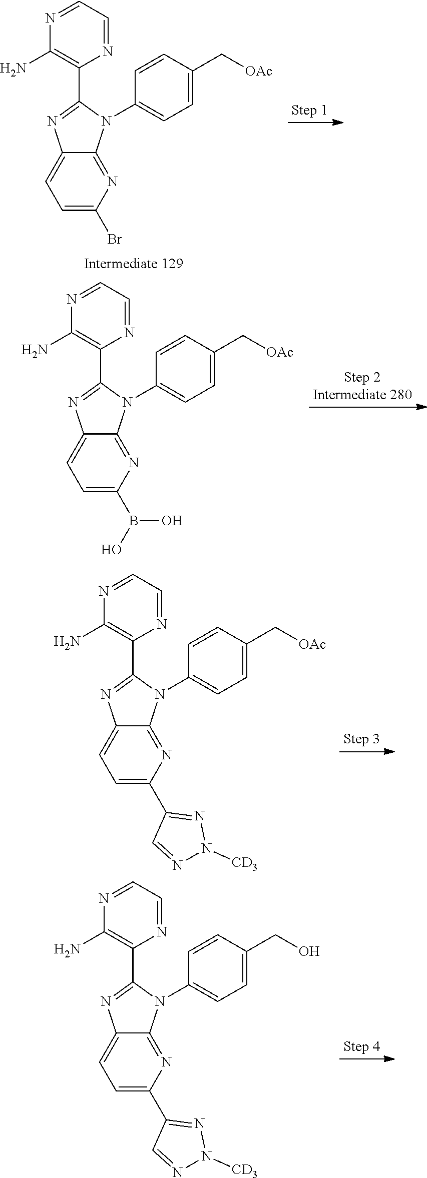

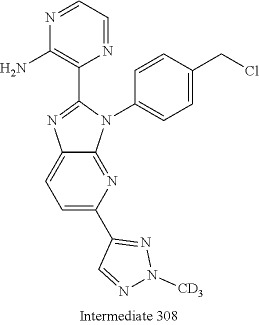 [SMILES: [2H]C([2H])([2H])n1ncc(-c2ccc3nc(-c4nccnc4N)n(-c4ccc(CCl)cc4)c3n2)n1]

### Step 1. (3-(4-(Acetoxymethyl)phenyl)-2-(3-aminopyrazin-2-yl)-3H-imidazo[4,5-b]pyridin-5-yl)boronic acid

A mixture of Intermediate 129, 4,4,4′,4′,5,5,5′,5′-octamethyl-2,2′-bi(1,3,2-dioxaborolane) (578 mg, 2.28 mmol), Pd(dppf)Cl₂ (83.3 mg, 114 μmol) and KOAc (335 mg, 3.41 mmol) in 1,4-dioxane (5 mL) was degassed and purged with N₂ three times. The mixture was stirred at 80° C. for 2 hr under N₂ atmosphere. (3-(4-(Acetoxymethyl)phenyl)-2-(3-aminopyrazin-2-yl)-3H-imidazo[4,5-b]pyridin-5-yl)boronic acid was obtained as a black liquid, which was used in the next step without work-up and purification. MS: m/z=405.3 [M+H]r.

### Step 2: 4-(2-(3-Aminopyrazin-2-yl)-5-(2-(methyl-d₃)-2H-1,2,3-triazol-4-yl)-3H-imidazo[4,5-b]pyridin-3-yl)benzyl acetate

A mixture of (3-(4-(acetoxymethyl)phenyl)-2-(3-aminopyrazin-2-yl)-3H-imidazo[4,5-b]pyridin-5-yl)boronic acid (460 mg, 1.14 mmol), Intermediate 280 (188 mg, 1.14 mmol), Cs₂CO₃ (1.11 g, 3.41 mmol) and Pd(dppf)Cl₂ (83.3 mg, 114 μmol) in 1,4-dioxane (10 mL) and H₂O (2 mL) was degassed and purged with N₂ three times. The mixture was stirred at 80° C. for 2 hr under N₂ atmosphere. The reaction mixture was quenched with H₂O (20 mL) and extracted with CH₂Cl₂ (20 mL×2). The combined organic layers were washed with brine (20 mL), dried over Na₂SO₄, filtered and concentrated under reduced pressure. After purified by silica gel flash chromatography (Eluent of 10%˜38% EtOAc in petroleum ether), 4-(2-(3-aminopyrazin-2-yl)-5-(2-(methyl-d₃)-2H-1,2,3-triazol-4-yl)-3H-imidazo[4,5-b]pyridin-3-yl)benzyl acetate (240 mg, yield: 45% for two steps) was obtained as a yellow solid. MS: m/z=445.2 [M+H]+. ¹H NMR (400 MHz, Dimethylsulfoxide-d₆) δ 8.35 (d, 1=8.0 Hz, 1H), 8.09-8.00 (m, 2H), 7.97 (d, J=8.4 Hz, 1H), 7.77 (br s, 2H), 7.54-7.48 (m, 3H), 7.45-7.41 (m, 2H), 5.19 (s, 2H), 2.13 (s, 3H).

### Step 3: (4-(2-(3-Aminopyrazin-2-yl)-5-(2-(methyl-d₃)-2H-1,2,3-triazol-4-yl)-3H-imidazo[4,5-b]pyridin-3-yl)phenyl)methanol

To a solution of 4-(2-(3-aminopyrazin-2-yl)-5-(2-(methyl-d₃)-2H-1,2,3-triazol-4-yl)-3H-imidazo[4,5-b]pyridin-3-yl)benzyl acetate (240 mg, 540 μmol) in THF (5 mL), MeOH (5 mL) and H₂O (5 mL) was added K₂CO₃ (224 mg, 1.62 mmol). The mixture was stirred at 25° C. for 1 hr. The reaction mixture was quenched with H₂O (20 mL) at 25° C. and extracted with CH₂Cl₂ (25 mL×2). The combined organic layers were washed with brine (30 mL), dried over Na₂SO₄, filtered and concentrated under reduced pressure. (4-(2-(3-Aminopyrazin-2-yl)-5-(2-(methyl-d₃)-2H-1,2,3-triazol-4-yl)-3H-imidazo[4,5-b]pyridin-3-yl)phenyl)methanol (200 mg, yield: 70%) was obtained as a yellow solid. MS: m/z=403.1 [M+H]+. ¹H NMR (400 MHz, Dimethylsulfoxide-d₆) δ 8.34 (d, J=8.4 Hz, 1H), 8.07-7.98 (m, 2H), 7.96 (d, J=8.0 Hz, 1H), 7.73 (br s, 2H), 7.53 (d, J=2.4 Hz, 1H), 7.46-7.42 (m, 2H), 7.39-7.34 (m, 2H), 5.34 (br t, J=5.6 Hz, 1H), 4.61 (d, J=5.6 Hz, 2H).

### Step 4: 3-(3-(4-(Chloromethyl)phenyl)-5-(2-(methyl-d₃)-2H-1,2,3-triazol-4-yl)-3H-imidazo[4,5-b]pyridin-2-yl)pyrazin-2-amine

To a solution of (4-(2-(3-aminopyrazin-2-yl)-5-(2-(methyl-d₃)-2H-1,2,3-triazol-4-yl)-3H-imidazo[4,5-b]pyridin-3-yl)phenyl)methanol (200 mg, 497 μmol) in CH₂Cl₂ (3 mL) was added SOCl₂ (296 mg, 2.49 mmol). The mixture was stirred at 40° C. for 0.5 hr. The reaction was concentrated under reduced pressure to give 3-(3-(4-(chloromethyl)phenyl)-5-(2-(methyl-d₃)-2H-1,2,3-triazol-4-yl)-3H-imidazo[4,5-b]pyridin-2-yl)pyrazin-2-amine (Intermediate 308, 220 mg, HCl salt) as a brown solid. MS: m/z=421.1, 423.1 [M+H]⁺.

### Intermediate 309: tert-Butyl N-tert-butoxycarbonyl-N-(4-formylisoxazol-3-yl)carbamate

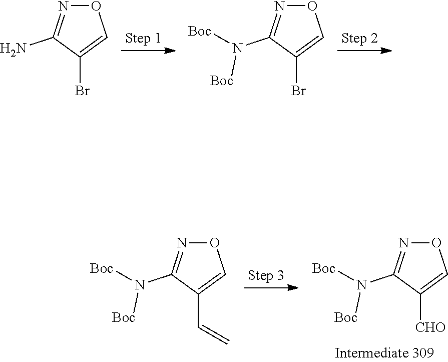 [SMILES: Nc1nocc1Br ; CC(C)(C)OC(=O)N(C(=O)OC(C)(C)C)c1nocc1Br ; C=Cc1conc1N(C(=O)OC(C)(C)C)C(=O)OC(C)(C)C ; CC(C)(C)OC(=O)N(C(=O)OC(C)(C)C)c1nocc1C=O]

### Step 1; tert-Butyl N-(4-bromoisoxazol-3-yl)-N-tert-butoxycarbonyl-carbamate

To a solution of 4-bromoisoxazol-3-amine (1 g, 6.14 mmol) in CH₂Cl₂ (10 mL) were added Boc₂O (3.35 g, 15.3 mmol), DMAP (75.0 mg, 614 μmol) and TEA (3.42 mL, 24.5 mmol). The mixture was degassed and purged with N₂ three times and stirred at 25° C. for 2 hr under N₂ atmosphere. The reaction mixture was quenched with CH₂Cl₂ (30 mL), washed with H₂O (30 mL×3), dried over Na₂SO₄, filtered and concentrated under reduced pressure. tert-Butyl N-(4-bromoisoxazol-3-yl)-N-tert-butoxycarbonyl-carbamate (2.40 g, crude) was obtained as a brown solid, which was used in the next step directly. MS: m/z=206.9, 208.9 [M−156+H]⁺. ¹H NMR (400 MHz, Chloroform-d) δ 8.43 (s, 1H), 1.44 (s, 18H).

### Step 2. tert-Butyl N-tert-butoxycarbonyl-N-(4-vinylisoxazol-3-yl)carbamate

To a solution of tert-butyl N-(4-bromoisoxazol-3-yl)-N-tert-butoxycarbonyl-carbamate (2.40 g, 6.61 mmol) in 1,4-dioxane (30 mL) and H₂O (6 mL) were added Pd(dppf)Cl₂ (484 mg, 661 μmol), Cs₂CO₃ (6.46 g, 19.8 mmol) and 4,4,5,5-tetramethyl-2-vinyl-1,3,2-dioxaborolane (2.24 mL, 13.2 mmol). The mixture was degassed and purged with N₂ for 3 times and stirred at 90° C. for 2 hr. The reaction mixture was quenched with EtOAc (100 mL), washed with brine (100 mL×3), dried over Na₂SO₄, filtered and concentrated under reduced pressure. After purified by silica gel flash chromatography (Eluent of 0%˜10% EtOAc in petroleum ether), tert-butyl N-tert-butoxycarbonyl-N-(4-vinylisoxazol-3-yl)carbamate (1.26 g, yield: 58% for two steps) was obtained as a pink oil. MS: m/z=643.2 [2M+Na]⁺. ¹H NMR (400 MHz, Chloroform-d) δ 8.42 (s, 1H), 6.33 (dd, J=18.0, 11.2 Hz, 1H), 5.54 (d, J=18.0 Hz, 1H), 5.34 (d, J=11.2 Hz, 1H), 1.42 (s, 18H)

### Step 3: tert-Butyl N-tert-butoxycarbonyl-N-(4-formylisoxazol-3-yl)carbamate

To a solution of tert-butyl N-tert-butoxycarbonyl-N-(4-vinylisoxazol-3-yl)carbamate (1.12 g, 3.61 mmol) in THF (20 mL) were added NaIO₄ (1.54 g, 7.22 mmol) and K₂OsO₄·2H₂O (133 mg, 361 μmol). The mixture was degassed and purged with N₂ three times and stirred at 0° C. for 2 hr under N₂. The reaction mixture was quenched with Sat.Na₂SO₃ (30 mL) at 0° C. and extracted with EtOAc (30 mL×3). The combined organic layers were washed with brine (50 mL×2), dried over Na₂SO₄, filtered and concentrated under reduced pressure. After purified by silica gel flash chromatography (Eluent of 0%˜10% EtOAc in petroleum ether), tert-butyl N-tert-butoxycarbonyl-N-(4-formylisoxazol-3-yl)carbamate (Intermediate 309, 178 mg, yield: 16%) was obtained as a white solid. ¹H NMR (400 MHz, Chloroform-d) δ 9.93 (s, 1H), 8.97 (s, 1H), 1.45 (s, 18H).

### Intermediate 310: 4-(3-(4-(Chloromethyl)phenyl)-5-(5-fluoropyridin-2-yl)-3H-imidazo[4,5-b]pyridin-2-yl)isoxazol-3-amine

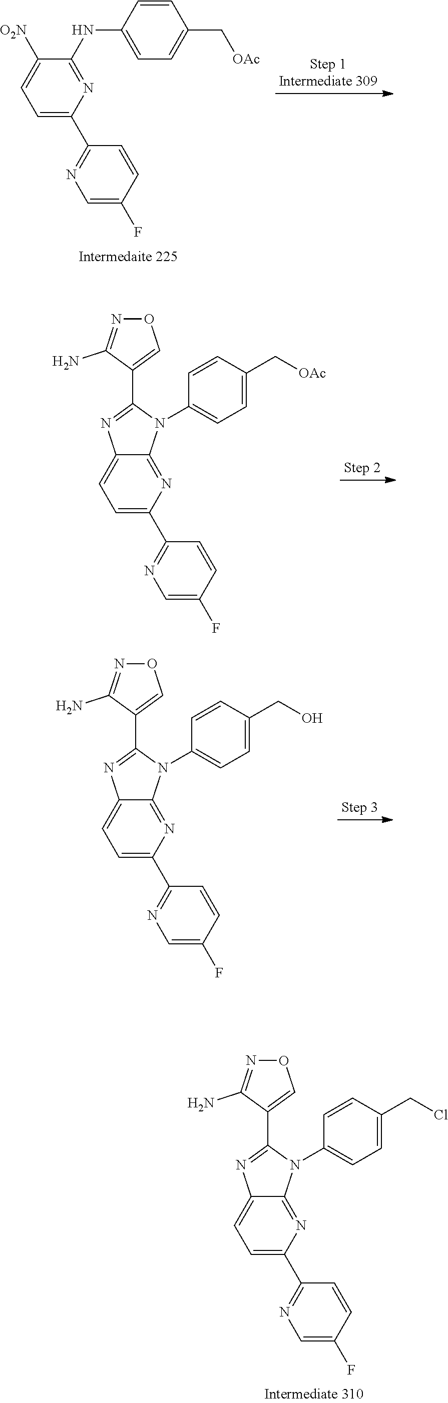 [SMILES: CC(=O)OCc1ccc(Nc2nc(-c3ccc(F)cn3)ccc2[N+](=O)[O-])cc1 ; CC(=O)OCc1ccc(-n2c(-c3conc3N)nc3ccc(-c4ccc(F)cn4)nc32)cc1 ; Nc1nocc1-c1nc2ccc(-c3ccc(F)cn3)nc2n1-c1ccc(CO)cc1 ; Nc1nocc1-c1nc2ccc(-c3ccc(F)cn3)nc2n1-c1ccc(CCl)cc1]

### Step 1: 4-(2-(3-Aminoisoxazol-4-yl)-5-(5-fluoropyridin-2-yl)-3H-imidazo[4,5-b]pyridin-3-yl)benzyl acetate

To a solution of Intermediate 225 (184 mg, 480 μmol) in DMSO (5 mL) were added Na₂S₂O₄ (251 mg, 1.44 mmol) and Intermediate 309 (150 mg, 480 μmol). The mixture was degassed and purged with N₂ three times and stirred at 100° C. for 2 hr under N₂. The reaction mixture was quenched with H₂O (20 mL) and extracted with CH₂Cl₂ (10 mL×3). The combined organic layers were washed with brine (20 mL×2), dried over Na₂SO₄, filtered and concentrated under reduced pressure. The residue was dissolved in CH₂Cl₂ (5 mL) and then TFA (1 mL) was added. The result mixture was stirred at 25° C. for 1 hr. The reaction mixture was quenched with H₂O (10 mL). The pH of the mixture was adjusted to pH to 8 with Sat. aq.NaHCO₃. The mixture was extracted with CH₂Cl₂ (10 mL×3). The combined organic layers were washed with brine (20 mL×2), dried over Na₂SO₄, filtered and concentrated under reduced pressure. After purified by silica gel flash chromatography (Eluent of 0%˜30% EtOAc in petroleum ether), 4-(2-(3-aminoisoxazol-4-yl)-5-(5-fluoropyridin-2-yl)-3H-imidazo[4,5-b]pyridin-3-yl)benzyl acetate (57.0 mg, yield: 25%) was obtained as a yellow solid. MS: m/z=445.0 [M+H]⁺. ¹H NMR (400 MHz, Chloroform-d) δ 8.48 (d, 1=2.4 Hz, 1H), 8.44 (d, J=8.4 Hz, 1H), 8.26 (dd, J=8.8, 4.8 Hz, 1H), 8.15 (d, J=8.4 Hz, 1H), 7.66 (d, J=8.4 Hz, 2H), 7.51 (d, J=8.4 Hz, 2H), 7.44-7.38 (m, 2H), 5.84 (br s, 2H), 5.30 (s, 2H), 2.21 (s, 3H). ¹⁹F NMR (400 MHz, Chloroform-d) δ −127.668.

### Step 2: (4-(2-(3-Aminoisoxazol-4-yl)-5-(5-fluoropyridin-2-yl)-3H-imidazo[4,5-b]pyridin-3-yl)phenyl)methanol

To a solution of 4-(2-(3-Aminoisoxazol-4-yl)-5-(5-fluoropyridin-2-yl)-3H-imidazo[4,5-b]pyridin-3-yl)benzyl acetate (57 mg, 128) in THF (1 mL), MeOH (1 mL) and H₂O (1 mL) was added K₂CO₃ (35.5 mg, 257 μmol). The mixture was degassed and purged with N₂ three times and stirred at 25° C. for 0.5 hr under N₂. The reaction mixture was quenched with EtOAc (20 mL), washed with brine (10 mL×2), dried over Na₂SO₄, filtered and concentrated under reduced pressure. (4-(2-(3-Aminoisoxazol-4-yl)-5-(5-fluoropyridin-2-yl)-3H-imidazo[4,5-b]pyridin-3-yl)phenyl)methanol (51.6 mg) was obtained as a yellow solid, which was used in the next step directly. MS: m/z=403.0 [M+H]⁺.

### Step 3: 4-(3-(4-(Chloromethyl)phenyl)-5-(5-fluoropyridin-2-yl)-3H-imidazo[4,5-b]pyridin-2-yl)isoxazol-3-amine

To a solution of (4-(2-(3-aminoisoxazol-4-yl)-5-(5-fluoropyridin-2-yl)-3H-imidazo[4,5-b]pyridin-3-yl)phenyl)methanol (51.6 mg, 128 μmol) in CH₂Cl₂ (1 mL) was added SOCl₂ (153 mg, 1.28 mmol). The mixture was degassed and purged with N₂ for 3 times and stirred at 25° C. for 1 hr. The reaction mixture was concentrated under reduced pressure to give 4-(3-(4-(chloromethyl)phenyl)-5-(5-fluoropyridin-2-yl)-3H-imidazo[4,5-b]pyridin-2-yl)isoxazol-3-amine (Intermediate 310, 58.7 mg, HCl salt) as a yellow solid which was used into the next step directly. MS: m/z=420.8, 422.8 [M+H]⁺.

### Intermediate 311 & 312: 5-Bromo-2-(fluoromethoxy-d₂)pyridine & 5-Bromo-1-(fluoromethyl-d₂)pyridin-2(1H)-one

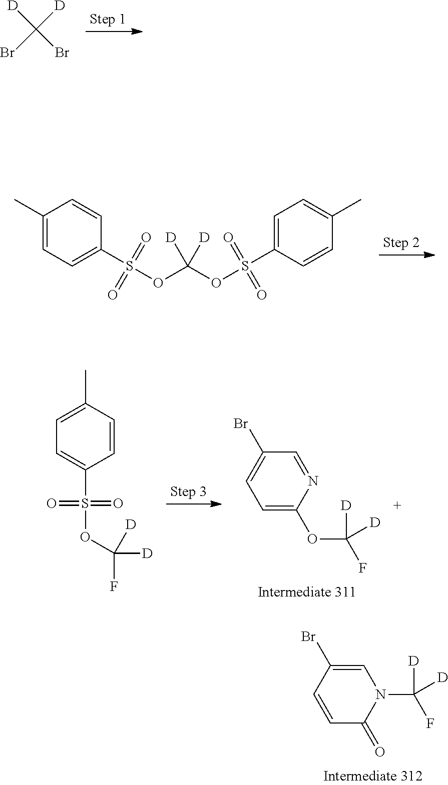

### Step 1: Methylene-d₂ bis(4-methylbenzenesulfonate)

To a solution of (tosyloxy)silver (52 g, 186 mmol) in MeCN (200 mL) was added dibromomethane-d₂ (14.9 g, 84.7 mmol). The mixture was stirred at 80° C. for 16 hr. The reaction mixture was filtered to remove all the salts. The filtrate was collected and concentrated under reduced pressure. The crude was triturated with CH₂Cl₂ (150 mL) at 25° C. for 3 hr. The filtrate was collected and concentrated under reduced pressure to give methylene-d₂ bis(4-methylbenzenesulfonate) (18 g, yield: 59%) was obtained as an off-white solid. MS: m/z=381.0 [M+Na]+. ¹H NMR (400 MHz, Dimethylsulfoxide-d₆) δ 7.60 (d, J=8.4 Hz, 4H), 7.40 (d, J=8.0 Hz, 4H), 2.42 (s, 6H).

### Step 2: Fluoromethyl-d₂ 4-methylbenzenesulfonate

To a solution of methylene-d₂ bis(4-methylbenzenesulfonate) (3.2 g, 8.93 mmol) in MeCN (80 mL) were added CsF (2.03 g, 13.4 mmol) and 3,6,9,12,15-pentaoxaheptadecane-1,17-diol (3.78 g, 13.4 mmol). The mixture was stirred at 80° C. for 16 hr. The residue was diluted with H₂O (10 mL) and extracted with CH₂Cl₂ (10 mL×3). The combined organic layers were washed with brine (5 mL×2), dried over Na₂SO₄, filtered and concentrated under reduced pressure. After purified by silica gel flash chromatography (Eluent of 0%˜5% EtOAc in petroleum ether), fluoromethyl-d₂ 4-methylbenzenesulfonate (18 g, yield: 48%) was obtained as a colorless oil. ¹H NMR (400 MHz, Dimethylsulfoxide-d₆) δ 7.85 (d, J=8.4 Hz, 2H), 7.51 (d, J=8.0 Hz, 2H), 2.43 (s, 3H). ¹⁹F NMR (400 MHz, Dimethylsulfoxide-d₆) δ −154.079.

### Step 3: 5-Bromo-2-(fluoromethoxy-d₂)pyridine & 5-Bromo-1-(fluoromethyl-d₂)pyridin-2(1H)-one

To a solution of fluoromethyl-d₂ 4-methylbenzenesulfonate (890 mg, 4.32 mmol), 5-bromopyridin-2-ol (751 mg, 4.32 mmol) in DMF (15 mL) was added K₂CO₃ (1.79 g, 13.0 mmol). The mixture was stirred at 50° C. for 2 hr. The residue was diluted with H₂O (10 mL) and extracted with CH₂Cl₂ (10 mL×3). The combined organic layers were washed with brine (5 mL×2), dried over Na₂SO₄, filtered and concentrated under reduced pressure. After purified by silica gel flash chromatography (Eluent of 0%˜15% EtOAc in petroleum ether), 5-bromo-2-(fluoromethoxy-d₂)pyridine (Intermediate 311, 490 mg, yield: 55%) was obtained as a colorless oil. ¹H NMR (400 MHz, Dimethylsulfoxide-d₆) δ 8.38 (d, J=2.4 Hz, 1H), 8.06 (dd, J=8.8, 2.4 Hz, 1H), 7.01 (d, J=8.8 Hz, 1H). ¹⁹F NMR (400 MHz, Dimethylsulfoxide-d₆) δ −156.130. 5-Bromo-1-(fluoromethyl-d₂)pyridin-2(1H)-one (Intermediate 312, 490 mg, yield: 19%) was obtained as a colorless oil. ¹H NMR (400 MHz, Dimethylsulfoxide-d₆) δ 8.19 (d, J=2.8 Hz, 1H), 7.62 (dd, J=10.0, 2.8 Hz, 1H), 6.47 (d, J=10.0 Hz, 1H). ¹⁹F NMR (400 MHz, Dimethylsulfoxide-d₆) δ −173.825.

### Intermediate 313: 3-(3-(4-(Chloromethyl)phenyl)-5-(6-(fluoromethoxy-d₂)pyridin-3-yl)-3H-imidazo[4,5-b]pyridin-2-yl)pyridin-2-amine

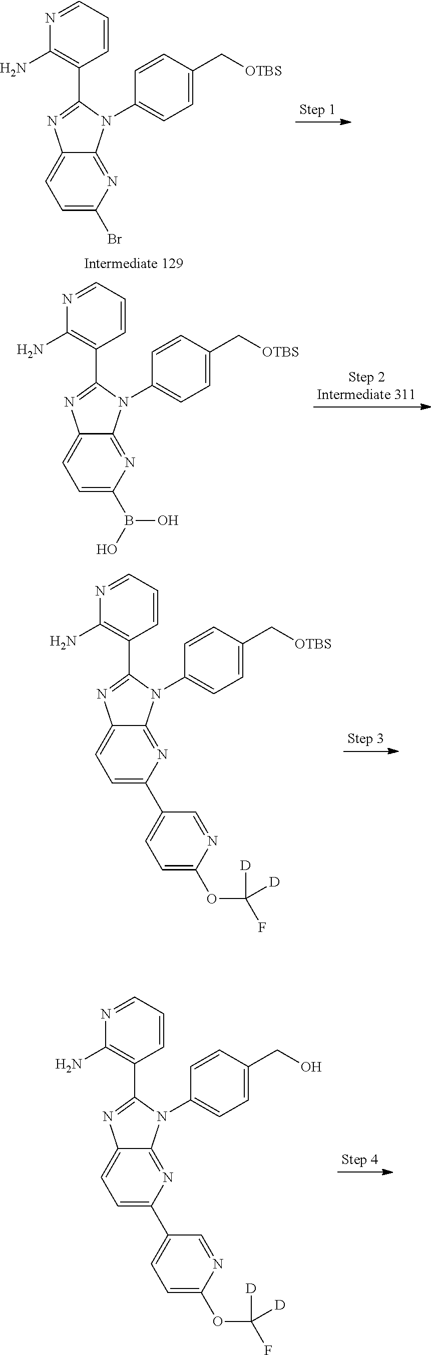

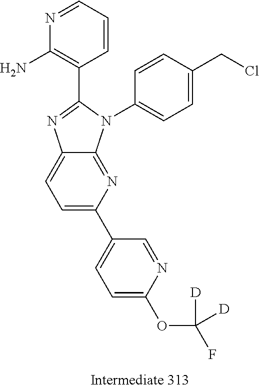 [SMILES: [2H]C([2H])(F)Oc1ccc(-c2ccc3nc(-c4cccnc4N)n(-c4ccc(CCl)cc4)c3n2)cn1]

### Step 1: (2-(2-Aminopyridin-3-yl)-3-(4-(((tert-butyldimethylsilyl)oxy)methyl)phenyl)-3H-imidazo[4,5-b]pyridin-5-yl)boronic acid

A mixture of Intermediate 129 (550 mg, 1.25 mmol), 4,4,4′,4′,5,5,5′,5′-octamethyl-2,2′-bi(1,3,2-dioxaborolane) (547 mg, 2.15 mmol), Pd(dppf)Cl₂ (78.8 mg, 108 μmol) and KOAc (317 mg, 3.23 mmol) in 1,4-dioxane (10 mL) was degassed and purged with N₂ three times. The mixture was stirred at 90° C. for 2 hr under N₂ atmosphere. (2-(2-Aminopyridin-3-yl)-3-(4-(((tert-butyldimethylsilyl)oxy)methyl)phenyl)-3H-imidazo[4,5-b]pyridin-5-yl)boronic acid was obtained as a black liquid, which was used in the next step without work-up and purification. MS: m/z=476.2 [M+H]⁺.

### Step 2: 3-(3-(4-(((tert-butyldimethylsilyl)oxy)methyl)phenyl)-5-(6-(fluoromethoxy-d₂)pyridin-3-yl)-3H-imidazo[4,5-b]pyridin-2-yl)pyridin-2-amine

A mixture of (2-(2-aminopyridin-3-yl)-3-(4-(((tert-butyldimethylsilyl)oxy)methyl)phenyl)-3H-imidazo[4,5-b]pyridin-5-yl)boronic acid (450 mg, 967 μmol), Intermediate 311 (197 mg, 967 μmol), Cs₂CO₃ (925 mg, 2.84 mmol) and Pd(dppf)Cl₂ (69.3 mg, 94.7 μmol) in 1,4-dioxane (10 mL) and H₂O (2 mL) was degassed and purged with N₂ three times. The mixture was stirred at 100° C. for 3 hr under N₂ atmosphere. The reaction mixture was quenched with H₂O (20 mL) and extracted with CH₂Cl₂ (15 mL×2). The combined organic layers were washed with brine (30 mL), dried over Na₂SO₄, filtered and concentrated under reduced pressure. After purified by silica gel flash chromatography (Eluent of 20%˜30% EtOAc in petroleum ether), 3-(3-(4-(((tert-butyldimethylsilyl)oxy)methyl)phenyl)-5-(6-(fluoromethoxy-d₂)pyridin-3-yl)-3H-imidazo[4,5-b]pyridin-2-yl)pyridin-2-amine (350 mg, yield: 63% for two steps) was obtained as a gray solid. MS: m/z=559.3 [M+H]⁺. ¹H NMR (400 MHz, Dimethylsulfoxide-d₆) δ 8.93-8.88 (m, 1H), 8.48-8.35 (m, 1H), 8.32-8.26 (m, 1H), 8.06-7.95 (m, 2H), 7.49-7.47 (m, 4H), 7.23-7.18 (m, 1H), 7.10-7.06 (m, 1H), 7.04-6.98 (m, 2H), 6.41-6.36 (m, 1H), 4.83 (s, 2H), 0.92 (s, 9H), 0.11 (s, 6H). ¹⁹F NMR (400 MHz, Dimethylsulfoxide-d₆) 6-155.567.

### Step 3: (4-(2-(2-Aminopyridin-3-yl)-5-(6-(fluoromethoxy-d₂)pyridin-3-yl)-3H-imidazo[4,5-b]pyridin-3-yl)phenyl)methanol

To a solution of 3-(3-(4-(((tert-butyldimethylsilyl)oxy)methyl)phenyl)-5-(6-(fluoromethoxy-d₂)pyridin-3-yl)-3H-imidazo[4,5-b]pyridin-2-yl)pyridin-2-amine (350 mg, 626 μmol) in THF (5 mL) was added TBAF (1 M in THF, 940 μL). The mixture was stirred at 25° C. for 1 hr. The mixture was stirred at 25° C. for 1 hr. The reaction mixture was quenched with H₂O (10 mL) at 25° C. and extracted with CH₂Cl₂ (10 mL×3). The combined organic layers were washed with brine (10 mL×2), dried over Na₂SO₄, filtered and concentrated under reduced pressure. (4-(2-(2-Aminopyridin-3-yl)-5-(6-(fluoromethoxy-d₂)pyridin-3-yl)-3H-imidazo[4,5-b]pyridin-3-yl)phenyl)methanol (270 mg, yield: 97%) was obtained as a gray solid.

MS: m/z=445.2 [M+H]⁺. ¹H NMR (400 MHz, Dimethylsulfoxide-d₆) δ 8.90 (d, J=2.0 Hz, 1H), 8.42 (dd, J=8.8, 2.4 Hz, 1H), 8.29 (d, J=8.8 Hz, 1H), 8.05-7.98 (m, 2H), 7.52-7.42 (m, 4H), 7.22 (dd, J=7.6, 1.2 Hz, 1H), 7.09 (d, J=8.4 Hz, 1H), 7.00 (br s, 2H), 6.42 (dd, J=7.6, 4.8 Hz, 1H), 5.43-5.33 (m, 1H), 4.60 (br d, J=5.6 Hz, 2H). ¹⁹F NMR (400 MHz, Dimethylsulfoxide-d₆) δ −155.567.

### Step 4: 3-(3-(4-(Chloromethyl)phenyl)-5-(6-(fluoromethoxy-d₂)pyridin-3-yl)-3H-imidazo[4,5-b]pyridin-2-yl)pyridin-2-amine

To a solution of (4-(2-(2-aminopyridin-3-yl)-5-(6-(fluoromethoxy-d₂)pyridin-3-yl)-3H-imidazo[4,5-b]pyridin-3-yl)phenyl)methanol (270 mg, 607 μmol) in CH₂Cl₂ (5 mL) was added SOCl₂ (145 mg, 1.21 mmol). The mixture was stirred at 25° C. for 1 hr. The reaction was concentrated under reduced pressure. 3-(3-(4-(Chloromethyl)phenyl)-5-(6-(fluoromethoxy-d₂)pyridin-3-yl)-3H-imidazo[4,5-b]pyridin-2-yl)pyridin-2-amine (Intermediate 313, 300 mg, HCl salt) was obtained as a brown solid. MS: m/z=463.0, 465.0 [M+H]⁺.

### Intermediate 314: 5-(2-(2-Aminopyridin-3-yl)-3-(4-(chloromethyl)phenyl)-3H-imidazo[4,5-b]pyridin-5-yl)-1-(fluoromethyl-d₂)pyridin-2(1H)-one

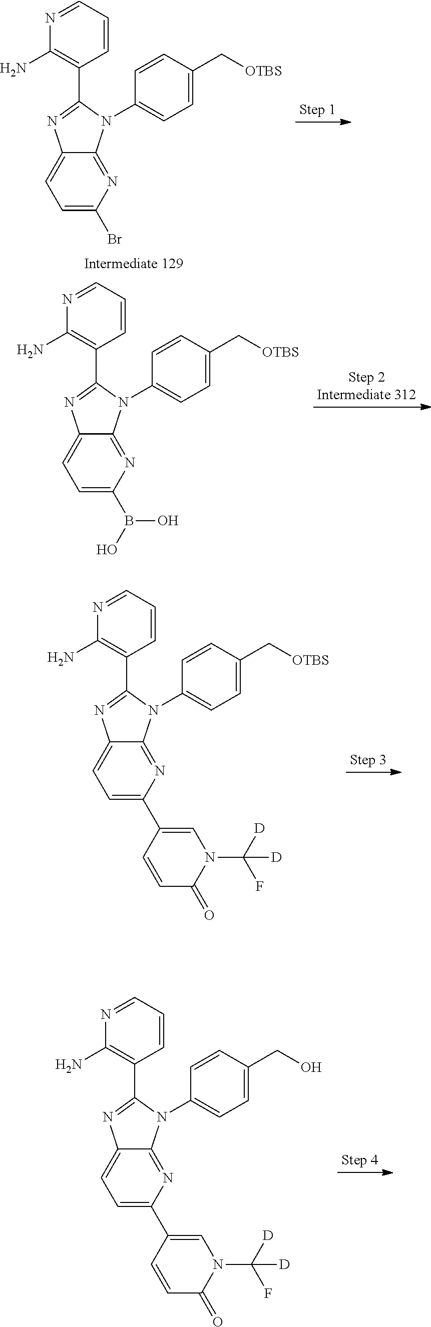

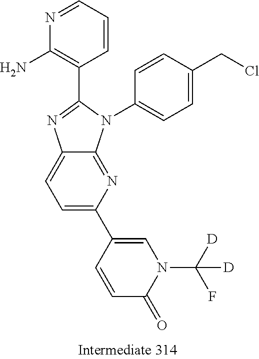 [SMILES: [2H]C([2H])(F)n1cc(-c2ccc3nc(-c4cccnc4N)n(-c4ccc(CCl)cc4)c3n2)ccc1=O]

### Step 1: (2-(2-Aminopyridin-3-yl)-3-(4-(((tert-butyldimethylsilyl)oxy)methyl)phenyl)-3H-imidazo[4,5-b]pyridin-5-yl)boronic acid

A mixture of Intermediate 29 (350 mg, 686 μmol), 4,4,4′,4′,5,5,5′,5′-octamethyl-2,2′-bi(1,3,2-dioxaborolane) (348 mg, 1.37 mmol), Pd(dppf)Cl₂ (50.2 mg, 68.6 μmol) and KOAc (202 mg, 2.06 mmol) in 1,4-dioxane (5 mL) was degassed and purged with N₂ three times. The mixture was stirred at 80° C. for 2 hr under N₂ atmosphere. (2-(2-Aminopyridin-3-yl)-3-(4-(((tert-butyldimethylsilyl)oxy)methyl)phenyl)-3H-imidazo[4,5-b]pyridin-5-yl)boronic acid was obtained as a black liquid, which was used in the next step without work-up and purification. MS: m/z=476.2 [M+H]⁺.

### Step 2: 5-(2-(2-Aminopyridin-3-yl)-3-(4-(((tert-butyldimethylsilyl)oxy)methyl)phenyl)-3H-imidazo[4,5-b]pyridin-5-yl)-1-(fluoromethyl-d₂)pyridin-2(1H)-one

A mixture of (2-(2-aminopyridin-3-yl)-3-(4-(((tert-butyldimethylsilyl)oxy)methyl)phenyl)-3H-imidazo[4,5-b]pyridin-5-yl)boronic acid (320 mg, 673 μmol), Intermediate 312 (140 mg, 673 μmol), Cs₂CO₃ (658 mg, 2.02 mmol) and Pd(dppf)Cl₂ (49.3 mg, 67.3 μmol) in 1,4-dioxane (5 mL) and H₂O (1 mL) was degassed and purged with N₂ three times. The mixture was stirred at 80° C. for 2 hr under N₂ atmosphere. The reaction mixture was quenched with H₂O (20 mL) and extracted with CH₂Cl₂ (20 mL×2). The combined organic layers were washed with brine (20 mL), dried over Na₂SO₄, filtered and concentrated under reduced pressure. After purified by silica gel flash chromatography (Eluent of 10%˜68% EtOAc in petroleum ether), 5-(2-(2-aminopyridin-3-yl)-3-(4-(((tert-butyldimethylsilyl)oxy)methyl)phenyl)-3H-imidazo[4,5-b]pyridin-5-yl)-1-(fluoromethyl-d₂)pyridin-2(1H)-one (175 mg, yield: 43% for two steps) was obtained as a yellow solid. MS: m/z=559.3 [M+H]⁺. ¹H NMR (400 MHz, Dimethylsulfoxide-d₆) δ 8.59 (d, J=2.0 Hz, 1H), 8.27 (d, J=8.4 Hz, 1H), 8.16 (dd, J=9.6, 2.4 Hz, 1H), 7.99 (dd, J=4.8, 1.6 Hz, 1H), 7.85 (d, J=8.4 Hz, 1H), 7.51-7.43 (m, 4H), 7.16 (dd, J=7.6, 1.6 Hz, 1H), 7.00 (br s, 2H), 6.59 (d, J=10.0 Hz, 1H), 6.38 (dd, J=7.6, 4.8 Hz, 1H), 4.82 (s, 2H), 0.92 (s, 9H), 0.10 (s, 6H). ¹⁹F NMR (400 MHz, Dimethylsulfoxide-d₆) δ −173.761.

### Step 3: 5-(2-(2-Aminopyridin-3-yl)-3-(4-(hydroxymethyl)phenyl)-3H-imidazo[4,5-b]pyridin-5-yl)-1-(fluoromethyl-d₂)pyridin-2(1H)-one

To a solution of 5-(2-(2-aminopyridin-3-yl)-3-(4-(((tert-butyldimethylsilyl)oxy)methyl)phenyl)-3H-imidazo[4,5-b]pyridin-5-yl)-1-(fluoromethyl-d₂)pyridin-2(1H)-one (175 mg, 313 μmol) in THF (5 mL) was added TBAF (0.5 mL, 1 M in THF). The mixture was stirred at 25° C. for 1 hr. The reaction mixture was quenched with H₂O (20 mL) at 25° C. and extracted with CH₂Cl₂ (20 mL×3). The combined organic layers were washed with brine (20 mL×3), dried over Na₂SO₄, filtered and concentrated under reduced pressure. 5-(2-(2-Aminopyridin-3-yl)-3-(4-(hydroxymethyl)phenyl)-3H-imidazo[4,5-b]pyridin-5-yl)-1-(fluoromethyl-d₂)pyridin-2(1H)-one (90 mg, yield. 65%) was obtained as a yellow solid. MS: m/z=445.1 [M+H]⁺. ¹H NMR (400 MHz, Dimethylsulfoxide-d₆) δ 8.61 (d, J=2.4 Hz, 1H), 8.26 (d, J=8.4 Hz, 1H), 8.16 (dd, J=9.6, 2.4 Hz, 1H), 8.02-7.93 (m, 1H), 7.86 (d, J=8.4 Hz, 1H), 7.51-7.41 (m, 4H), 7.23-7.15 (m, 1H), 6.96 (br s, 2H), 6.61 (d, J=9.6 Hz, 1H), 6.41 (dd, J=7.6, 4.8 Hz, 1H), 5.37 (br t, J=5.6 Hz, 1H), 4.60 (d, J=5.6 Hz, 2H).

### Step 4: 5-(2-(2-Aminopyridin-3-yl)-3-(4-(chloromethyl)phenyl)-3H-imidazo[4,5-b]pyridin-5-yl)-1-(fluoromethyl-d₂)pyridin-2(1H)-one

To a solution of 5-(2-(2-aminopyridin-3-yl)-3-(4-(hydroxymethyl)phenyl)-3H-imidazo[4,5-b]pyridin-5-yl)-1-(fluoromethyl-d₂)pyridin-2(1H)-one (90 mg, 202 μmol) in CH₂Cl₂ (3 mL) was added SOCl₂ (72 mg, 606 μmol). The mixture was stirred at 40° C. for 0.5 hr. The reaction was concentrated under reduced pressure to give 5-(2-(2-aminopyridin-3-yl)-3-(4-(chloromethyl)phenyl)-3H-imidazo[4,5-b]pyridin-5-yl)-1-(fluoromethyl-d₂)pyridin-2(1H)-one (Intermediate 314, 101 mg, HCl salt) was obtained as a yellow solid. MS: m/z=463.1, 465.1 [M+H]⁺.

### Intermediate 315: 4-Bromo-1-ethyl-1H-1,2,3-triazole

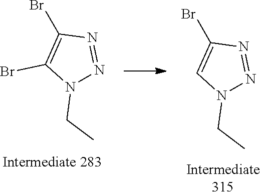

To a mixture of Intermediate 283 (680 mg, 2.67 mmol) in THF (2 mL) was added n-BuLi (2.5 M in hexane, 1.07 mL) at −70° C. under N₂, the mixture was stirred at −70° C. for 1 hr. The mixture was diluted with H₂O (20 mL) and extracted with EtOAc (20 mL×2). The combined organic layers were washed with brine (20 mL), dried over anhydrous Na₂SO₄, filtered and concentrated. 4-Bromo-1-ethyl-1H-1,2,3-triazole (Intermediate 315, 440 mg, 2.50 mmol, yield: 94%) was obtained as a yellow oil. MS: m/z=175.8, 177.8 [M+H]⁺. ¹H NMR (400 MHz, Dimethylsulfoxide-d₆) δ 8.42 (s, 1H), 4.40 (q, J=7.2 Hz, 2H), 1.42 (t, J=7.2 Hz, 3H).

### Intermediate 316: 3-(3-(4-(Chloromethyl)phenyl)-5-(1-ethyl-1H-1,2,3-triazol-4-yl)-3H-imidazo[4,5-b]pyridin-2-yl)pyridin-2-amine

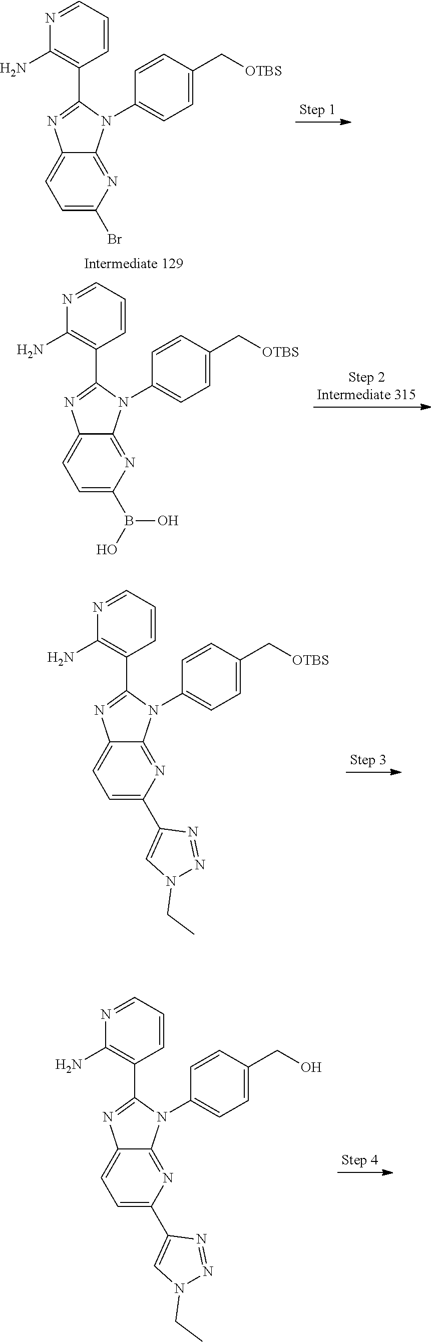

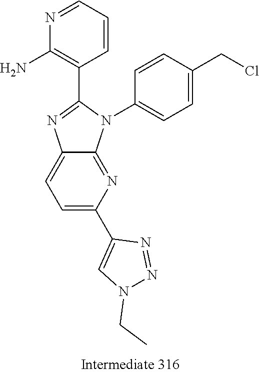 [SMILES: CCn1cc(-c2ccc3nc(-c4cccnc4N)n(-c4ccc(CCl)cc4)c3n2)nn1]

### Step 1: (2-(2-Aminopyridin-3-yl)-3-(4-(((tert-butyldimethylsilyl)oxy)methyl)phenyl)-3H-imidazo[4,5-b]pyridin-5-yl)boronic acid

A mixture of Intermediate 129 (550 mg, 1.25 mmol), 4,4,4′,4′,5,5,5′,5′-octamethyl-2,2′-bi(1,3,2-dioxaborolane) (547 mg, 2.15 mmol), Pd(dppf)Cl₂ (78.8 mg, 108 μmol) and KOAc (317 mg, 3.23 mmol) in 1,4-dioxane (10 mL) was degassed and purged with N₂ three times. The mixture was stirred at 90° C. for 2 hr under N₂ atmosphere. (2-(2-Aminopyridin-3-yl)-3-(4-(((tert-butyldimethylsilyl)oxy)methyl)phenyl)-3H-imidazo[4,5-b]pyridin-5-yl)boronic acid was obtained as a black liquid, which was used in the next step without work-up and purification. MS: m/z=476.2 [M+H]⁺.

### Step 2: 3-(3-(4-(((tert-Butyldimethylsilyl)oxy)methyl)phenyl)-5-(1-ethyl-1H-1,2,3-triazol-4-yl)-3H-imidazo[4,5-b]pyridin-2-yl)pyridin-2-amine

To a mixture of (2-(2-aminopyridin-3-yl)-3-(4-(((tert-butyldimethylsilyl)oxy)methyl)phenyl)-3H-imidazo[4,5-b]pyridin-5-yl)boronic acid (330 mg, 694 μmol) and Intermediate 315 (134 mg, 764 μmol) in 1,4-dioxane (6 mL) and H₂O (2 mL) were added Cs₂CO₃ (678 mg, 2.08 mmol) and Pd(dppf)Cl₂ (50.8 mg, 69.4 μmol). The mixture was stirred at 100° C. for 2 hr under N₂. The mixture was diluted with H₂O (20 mL) and extracted with EtOAc (20 mL×2). The combined organic layers were washed with brine (20 mL), dried over anhydrous Na₂SO₄, filtered and concentrated. After purified by silica gel flash chromatography (EtOAc in petroleum ether=0-50%), 3-(3-(4-(((tert-butyldimethylsilyl)oxy)methyl)phenyl)-5-(1-ethyl-1H-1,2,3-triazol-4-yl)-3H-imidazo[4,5-b]pyridin-2-yl)pyridin-2-amine (210 mg, 399 μmol, yield: 57%) was obtained as a yellow solid. MS: m/z=527.3 [M+H]⁺. ¹H NMR (400 MHz, Dimethylsulfoxide-d₆) δ 8.39 (s, 1H), 8.27 (d, J=8.0 Hz, 1H), 8.08 (d, J=8.0 Hz, 1H), 7.98 (dd, J=4.8, 1.6 Hz, 1H), 7.51-7.41 (m, 4H), 7.16 (dd, J=7.6, 1.6 Hz, 1H), 6.96 (br s, 2H), 6.37 (dd, J=7.6, 4.8 Hz, 1H), 4.82 (s, 2H), 4.42 (q, J=7.6 Hz, 2H), 1.44 (t, J=7.6 Hz, 3H), 0.93 (s, 9H), 0.11 (s, 6H).

### Step 3: (4-(2-(2-Aminopyridin-3-yl)-5-(1-ethyl-1H-1,2,3-triazol-4-yl)-3H-imidazo[4,5-b]pyridin-3-yl)phenyl)methanol

To a mixture of 3-(3-(4-(((tert-butyldimethylsilyl)oxy)methyl)phenyl)-5-(1-ethyl-1H-1,2,3-triazol-4-yl)-3H-imidazo[4,5-b]pyridin-2-yl)pyridin-2-amine (210 mg, 399 μmol) in THF (2 mL) was added TBAF (1 M in THF, 598 μL). The mixture was stirred at 20° C. for 1 hr. The mixture was diluted with H₂O (20 mL) and extracted with EtOAc (20 mL×2). The combined organic layers were washed with brine (10 mL), dried over anhydrous Na₂SO₄, filtered and concentrated. (4-(2-(2-Aminopyridin-3-yl)-5-(1-ethyl-1H-1,2,3-triazol-4-yl)-3H-imidazo[4,5-b]pyridin-3-yl)phenyl)methanol (164 mg, 398 μmol) was obtained as a yellow solid. MS: m/z=413.2 [M+H]⁺. ¹H NMR (400 MHz, Dimethylsulfoxide-d₆) δ 8.40 (s, 1H), 8.27 (d, J=8.0 Hz, 1H), 8.08 (d, J=8.4 Hz, 1H), 8.01-7.97 (m, 1H), 7.50-7.47 (m, 2H), 7.44-7.41 (m, 2H), 7.20 (dd, J=7.6, 2.0 Hz, 1H), 6.93 (br s, 2H), 6.41 (dd, J=7.6, 4.8 Hz, 1H), 5.40 (t, J=5.6 Hz, 1H), 4.60 (d, J=5.6 Hz, 2H), 4.43 (q, J=7.6 Hz, 2H), 1.45 (t, J=7.6 Hz, 3H).

### Step 4: 3-(3-(4-(Chloromethyl)phenyl)-5-(1-ethyl-1H-1,2,3-triazol-4-yl)-3H-imidazo[4,5-b]pyridin-2-yl)pyridin-2-amine

To a mixture of (4-(2-(2-aminopyridin-3-yl)-5-(1-ethyl-1H-1,2,3-triazol-4-yl)-3H-imidazo[4,5-b]pyridin-3-yl)phenyl)methanol (160 mg, 388 μmol) in CH₂Cl₂ (2 mL) was added SOCl₂ (28.2 μL, 388 μmol). The mixture was stirred at 20° C. for 1 hr. The mixture was concentrated to give 3-(3-(4-(chloromethyl)phenyl)-5-(1-ethyl-1H-1,2,3-triazol-4-yl)-3H-imidazo[4,5-b]pyridin-2-yl)pyridin-2-amine (Intermediate 316, 167 mg, 388 μmol) was obtained as a yellow solid. MS: m/z=431.2, 433.1 [M+H]⁺.

### Intermediate 317: 2-(4-Bromo-2H-1,2,3-triazol-2-yl)acetonitrile

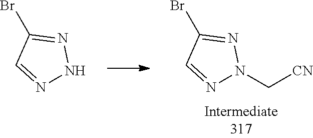 [SMILES: Brc1cn[nH]n1 ; N#CCn1ncc(Br)n1]

To a solution of 4-bromo-2H-1,2,3-triazole (300 mg, 2.03 mmol) and 2-bromoacetonitrile (121 mg, 1.01 mmol) in DMF (5 mL) was added K₂CO₃ (841 mg, 6.08 mmol). The mixture was stirred at 25° C. for 2 hr under N₂. The reaction mixture was quenched with NaHSO₃ (10 mL), diluted with H₂O (10 mL), and extracted with CH₂Cl₂ (5 mL×2). The combined organic layers were washed with brine (10 mL), dried over Na₂SO₄, filtered and concentrated under reduced pressure. 2-(4-Bromo-2H-1,2,3-triazol-2-yl)acetonitrile (Intermediate 317, 379 mg) was obtained as a light yellow oil. ¹H NMR (400 MHz, Dimethylsulfoxide-d₆) δ 8.18 (s, 1H), 5.94 (s, 2H).

### Intermediate 318: 2-(4-(2-(2-Aminopyridin-3-yl)-3-(4-(chloromethyl)phenyl)-3H-imidazo[4,5-b]pyridin-5-yl)-2H-1,2,3-triazol-2-yl)acetonitrile

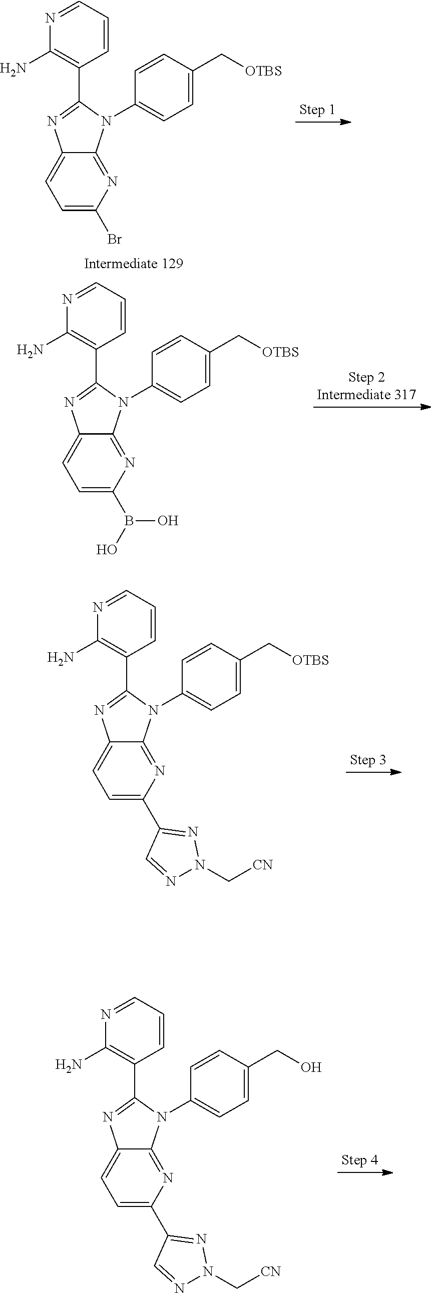

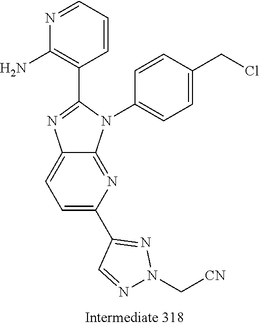 [SMILES: N#CCn1ncc(-c2ccc3nc(-c4cccnc4N)n(-c4ccc(CCl)cc4)c3n2)n1]

### Step 1: (2-(2-Aminopyridin-3-yl)-3-(4-(((tert-butyldimethylsilyl)oxy)methyl)phenyl)-3H-imidazo[4,5-b]pyridin-5-yl)boronic acid

To a solution of Intermediate 129 (300 mg, 588 μmol) and 4,4,4′,4′,5,5,5′,5′-octamethyl-2,2′-bi(1,3,2-dioxaborolane) (299 mg, 1.18 mmol) in 1,4-dioxane (1 mL) were added KOAc (173 mg, 1.76 mmol) and Pd(dppf)Cl₂ (43.0 mg, 58.8 μmol). The mixture was stirred at 85° C. for 2 hr under N₂, (2-(2-aminopyridin-3-yl)-3-(4-(((tert-butyldimethylsilyl)oxy)methyl)phenyl)-3H-imidazo[4,5-b]pyridin-5-yl)boronic acid was obtained as a light yellow liquid, which was used in the next step without work up. MS: m/z=476.3 [M+H]⁺.

### Step 2: 2-(4-(2-(2-Aminopyridin-3-yl)-3-(4-(((tert-butyldimethylsilyl)oxy)methyl)phenyl)-3H-imidazo[4,5-b]pyridin-5-yl)-2H-1,2,3-triazol-2-yl)acetonitrile

To a solution of 3-(5-bromo-3-(4-(((tert-butyldimethylsilyl)oxy)methyl)phenyl)-3H-imidazo[4,5-h]pyridin-2-yl)pyridin-2-amine (279 mg, 588 μmol) and Intermediate 317 (110 mg, 588 μmol) in H₂O (0.2 mL) and 1,4-dioxane (1 mL) were added Cs₂CO₃ (574 mg, 1.76 mmol) and Pd(dppf)Cl₂ (43.0 mg, 58.8 μmol). The mixture was stirred at 100° C. for 2 hr under N₂ atmosphere. The reaction mixture was quenched with H₂O (20 mL) and extracted with CH₂Cl₂ (15 mL×2). The combined organic layers were washed with brine (30 mL), dried over Na₂SO₄, filtered and concentrated under reduced pressure. After purified by silica gel flash chromatography (Eluent of 0%˜50% ethyl acetate in petroleum ether), 2-(4-(2-(2-aminopyridin-3-yl)-3-(4-(((tert-butyldimethylsilyl)oxy)methyl)phenyl)-3H-imidazo[4,5-b]pyridin-5-yl)-2H-1,2,3-triazol-2-yl)acetonitrile (170 mg, yield: 54% for two steps) was obtained as a light yellow solid. MS: m/z=538.3 [M+H]⁺.

### Step 3: 2-(4-(2-(2-Aminopyridin-3-yl)-3-(4-(hydroxymethyl)phenyl)-3H-imidazo[4,5-b]pyridin-5-yl)-2H-1,2,3-triazol-2-yl)acetonitrile

To a solution of 2-(4-(2-(2-aminopyridin-3-yl)-3-(4-(((tert-butyldimethylsilyl)oxy)methyl)phenyl)-3H-imidazo[4,5-b]pyridin-5-yl)-2H-1,2,3-triazol-2-yl)acetonitrile (170 mg, 316 μmol) in THF (2 mL) were added TBAF (1 M in THF, 411 μL). The mixture was stirred at 25° C. for 0.5 hr. H₂O (10 mL) was added and the aqueous was extracted with EtOAc (10 mL×2). The combined organic layers were dried over anhydrous Na₂SO₄, filtered and concentrated. 2-(4-(2-(2-Aminopyridin-3-yl)-3-(4-(hydroxymethyl)phenyl)-3H-imidazo[4,5-b]pyridin-5-yl)-2H-1,2,3-triazol-2-yl)acetonitrile (134 mg) was obtained as light yellow oil. MS: m/z=424.2 [M+H]⁺.

### Step 4: 2-(4-(2-(2-Aminopyridin-3-yl)-3-(4-(chloromethyl)phenyl)-3H-imidazo[4,5-b]pyridin-5-yl)-2H-1,2,3-triazol-2-yl)acetonitrile

To a solution of 2-(4-(2-(2-aminopyridin-3-yl)-3-(4-(hydroxymethyl)phenyl)-3H-imidazo[4,5-b]pyridin-5-yl)-2H-1,2,3-triazol-2-yl)acetonitrile (134 mg, 316 μmol) in CH₂Cl₂ (1 mL) was added SOCl₂ (68.9 μL). The mixture was stirred at 25° C. for 1 hr. The reaction was concentrated to give 2-(4-(2-(2-aminopyridin-3-yl)-3-(4-(chloromethyl)phenyl)-3H-imidazo[4,5-b]pyridin-5-yl)-2H-1,2,3-triazol-2-yl)acetonitrile (Intermediate 318, 140 mg) was obtained as a light yellow solid. MS: m/z=442.1, 444.1[M+H]⁺

### Intermediate 319 & 320: 4-(2-(3-Aminopyrazin-2-yl)-5-(2-(fluoromethyl)-2H-1,2,3-triazol-4-yl)-3H-imidazo[4,5-b]pyridin-3-yl)benzyl acetate & 4-(2-(3-Aminopyrazin-2-yl)-5-(1-(fluoromethyl)-1H-1,2,3-triazol-4-yl)-3H-imidazo[4,5-b]pyridin-3-yl)benzyl acetate

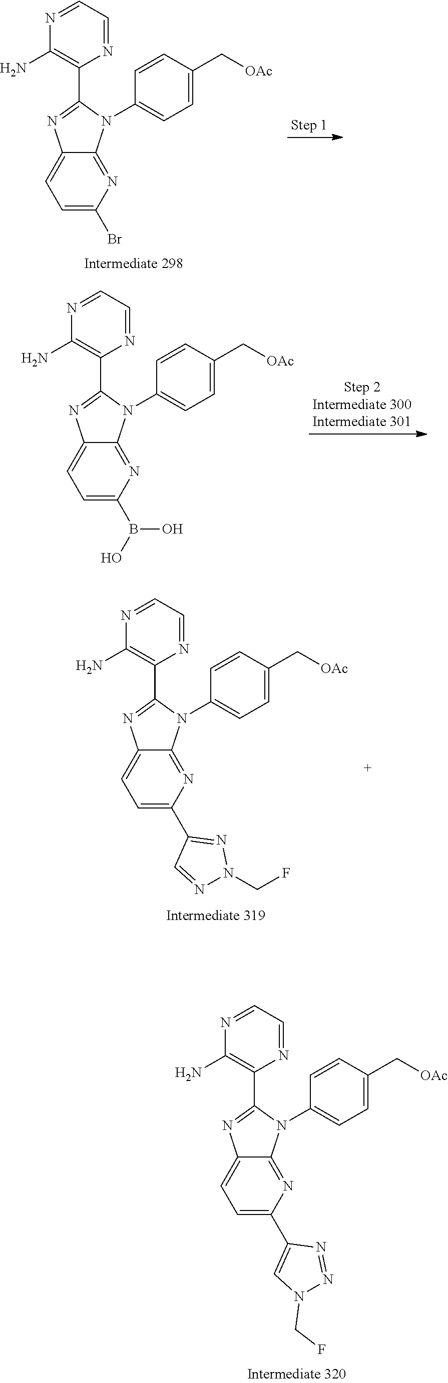 [SMILES: CC(=O)OCc1ccc(-n2c(-c3nccnc3N)nc3ccc(Br)nc32)cc1 ; CC(=O)OCc1ccc(-n2c(-c3nccnc3N)nc3ccc(B(O)O)nc32)cc1 ; CC(=O)OCc1ccc(-n2c(-c3nccnc3N)nc3ccc(-c4cnn(CF)n4)nc32)cc1 ; CC(=O)OCc1ccc(-n2c(-c3nccnc3N)nc3ccc(-c4cn(CF)nn4)nc32)cc1]

### Step 1: (3-(4-(Acetoxymethyl)phenyl)-2-(3-aminopyrazin-2-yl)-3H-imidazo[4,5-b]pyridin-5-yl)boronic acid

A mixture of Intermediate 298 (500 mg, 1.14 mmol), 4,4,4′,4′,5,5,5′,5′-octamethyl-2,2′-bi(1,3,2-dioxaborolane) (578 mg, 2.28 mmol), KOAc (335 mg, 3.41 mmol) and Pd(dppf)Cl₂ (83.3 mg, 114 μmol) in 1,4-dioxane (5 mL) was degassed and purged with N₂ three times. The mixture was stirred at 90° C. for 2 hr under N₂ atmosphere. (3-(4-(Acetoxymethyl)phenyl)-2-(3-aminopyrazin-2-yl)-3H-imidazo[4,5-b]pyridin-5-yl)boronic acid was obtained as a black liquid, which was used in the next step without work-up and purification. MS: m/z=405.2 [M+H]⁺

### Step 2: 4-(2-(3-Aminopyrazin-2-yl)-5-(2-(fluoromethyl)-2H-1,2,3-triazol-4-yl)-3H-imidazo[4,5-b]pyridin-3-yl)benzyl acetate & 4-(2-(3-Aminopyrazin-2-yl)-5-(1-(fluoromethyl)-1H-1,2,3-triazol-4-yl)-3H-imidazo[4,5-b]pyridin-3-yl)benzyl acetate

A mixture of (3-(4-(acetoxymethyl)phenyl)-2-(3-aminopyrazin-2-yl)-3H-imidazo[4,5-b]pyridin-5-yl)boronic acid (450 mg, 1.11 mmol), Intermediate 300 (200 mg, 1.11 mmol, contained Intermediate 301), Pd(dppf)Cl₂ (81.5 mg, 111 μmol), Cs₂CO₃ (1.09 g, 3.34 mmol) in 1,4-dioxane (10 mL) and H₂O (2.5 mL) was degassed and purged with N₂ three times. The mixture was stirred at 100° C. for 16 hr under N₂ atmosphere. The residue was diluted with H₂O (10 mL) and extracted with CH₂Cl₂ (10 mL×3). The combined organic layers were washed with brine (5 mL×2), dried over Na₂SO₄, filtered and concentrated under reduced pressure. After purified by prep-HPLC (column: Waters Xbridge BEH C18 250×50 mm×10 μm; mobile phase: [water (NH₄HCO₃)-ACN]; gradient: 33%-63% B over 10 min), 4-(2-(3-aminopyrazin-2-yl)-5-(2-(fluoromethyl)-2H-1,2,3-triazol-4-yl)-3H-imidazo[4,5-b]pyridin-3-yl)benzyl acetate (Intermediate 319, 200 mg, yield: 36% for two steps) was obtained as a gray solid. MS: m/z=460.1 [M+H]⁺. ¹H NMR (400 MHz, Dimethylsulfoxide-d₆) δ 8.41 (d, J=8.4 Hz, 1H), 8.35 (s, 1H), 8.09-7.98 (m, 2H), 7.78 (br s, 2H), 7.56-7.41 (m, 5H), 6.49 (d, J=51.6 Hz, 2H), 5.20 (s, 2H), 2.13 (s, 3H). ¹⁹F NMR (400 MHz, Dimethylsulfoxide-d₆) δ −166.385. 4-(2-(3-Aminopyrazin-2-yl)-5-(1-(fluoromethyl)-1H-1,2,3-triazol-4-yl)-3H-imidazo[4,5-b]pyridin-3-yl)benzyl acetate (Intermediate 320, 80 mg, yield: 15% for two steps) was also obtained as a gray solid. MS: m/z=460.1 [M+H]⁺. ¹H NMR (400 MHz, Dimethylsulfoxide-d₆) δ 8.77 (s, 1H), 8.39 (d, J=8.4 Hz, 1H), 8.16 (d, J=8.4 Hz, 1H), 8.02 (d, J=2.0 Hz, 1H), 7.78 (br s, 2H), 7.53-7.42 (m, 5H), 6.49 (d, J=50.4 Hz, 2H), 5.19 (s, 2H), 2.13 (s, 3H). ¹⁹F NMR (400 MHz, Dimethylsulfoxide-d₆) δ −166.760.

### Intermediate 321: 3-(3-(4-(Chloromethyl)phenyl)-5-(2-(fluoromethyl)-2H-1,2,3-triazol-4-yl)-3H-imidazo[4,5-b]pyridin-2-yl)pyrazin-2-amine

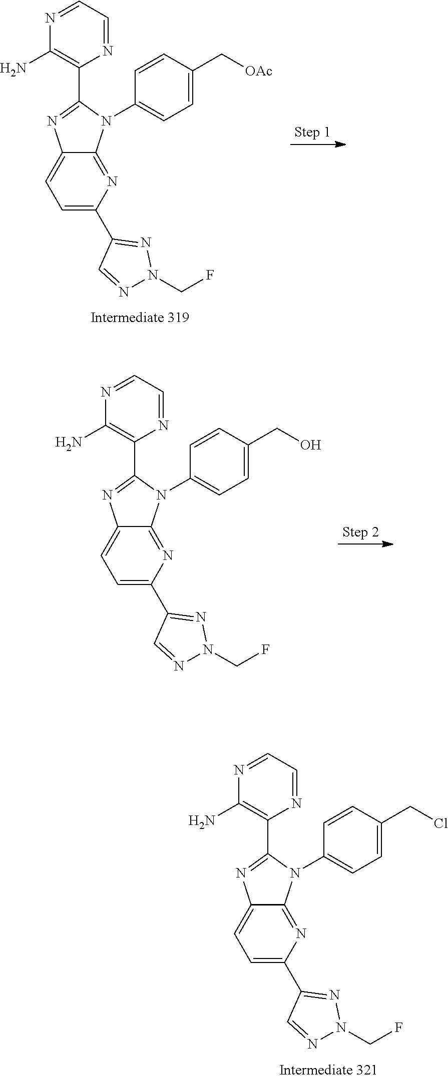 [SMILES: CC(=O)OCc1ccc(-n2c(-c3nccnc3N)nc3ccc(-c4cnn(CF)n4)nc32)cc1 ; Nc1nccnc1-c1nc2ccc(-c3cnn(CF)n3)nc2n1-c1ccc(CO)cc1 ; Nc1nccnc1-c1nc2ccc(-c3cnn(CF)n3)nc2n1-c1ccc(CCl)cc1]

### Step 1: (4-(2-(3-Aminopyrazin-2-yl)-5-(2-(fluoromethyl)-2H-1,2,3-triazol-4-yl)-3H-imidazo[4,5-b]pyridin-3-yl)phenyl)methanol

To a solution of Intermediate 319 (200 mg, 435 μmol) in MeOH (2 mL), THF (2 mL) and H₂O (0.5 mL) was added K₂CO₃ (180 mg, 1.31 mmol). The mixture was stirred at 25° C. for 0.5 hr. The residue was diluted with H₂O (8 mL) and extracted with CH₂Cl₂ (10 mL×3). The combined organic layers were washed with brine (5 mL×2), dried over Na₂SO₄, filtered and concentrated under reduced pressure. (4-(2-(3-Aminopyrazin-2-yl)-5-(2-(fluoromethyl)-2H-1,2,3-triazol-4-yl)-3H-imidazo[4,5-b]pyridin-3-yl)phenyl)methanol (160 mg, crude) was obtained as a yellow solid which was used into the next step directly. MS: m/z=417.9 [M+H]⁺. ¹H NMR (400 MHz, Dimethylsulfoxide-d₆) δ 8.40 (d, J=8.0 Hz, 1H), 8.33 (s, 1H), 8.08-7.99 (m, 2H), 7.74 (br s, 2H), 7.55 (d, J=2.4 Hz, 1H), 7.47-7.42 (m, 2H), 7.41-7.35 (m, 2H), 6.48 (d, J=51.6 Hz, 2H), 5.34 (br t, J=5.6 Hz, 1H), 4.61 (d, J=5.6 Hz, 2H). ¹⁹F NMR (400 MHz, Dimethylsulfoxide-d₆) δ −166.387.

### Step 2: 3-(3-(4-(Chloromethyl)phenyl)-5-(2-(fluoromethyl)-2H-1,2,3-triazol-4-yl)-3H-imidazo[4,5-b]pyridin-2-yl)pyrazin-2-amine

To a solution of (4-(2-(3-aminopyrazin-2-yl)-5-(2-(fluoromethyl)-2H-1,2,3-triazol-4-yl)-3H-imidazo[4,5-b]pyridin-3-yl)phenyl)methanol (200 mg, 479 μmol) in CH₂Cl₂ (5 mL) was added SOCl₂ (114 mg, 958 μmol). The reaction mixture was stirred at 40° C. for 1 hr. The reaction mixture was concentrated under reduced pressure to give 3-(3-(4-(chloromethyl)phenyl)-5-(2-(fluoromethyl)-2H-1,2,3-triazol-4-yl)-3H-imidazo[4,5-b]pyridin-2-yl)pyrazin-2-amine (Intermediate 321, 200 mg, crude, HCl salt) as a gray solid, which was used in the next step directly. MS: m/z=435.8, 437.8 [M+H]⁺.

### Intermediate 322: 3-(3-(4-(Chloromethyl)phenyl)-5-(1-(fluoromethyl)-1H-1,2,3-triazol-4-yl)-3H-imidazo[4,5-b]pyridin-2-yl)pyrazin-2-amine

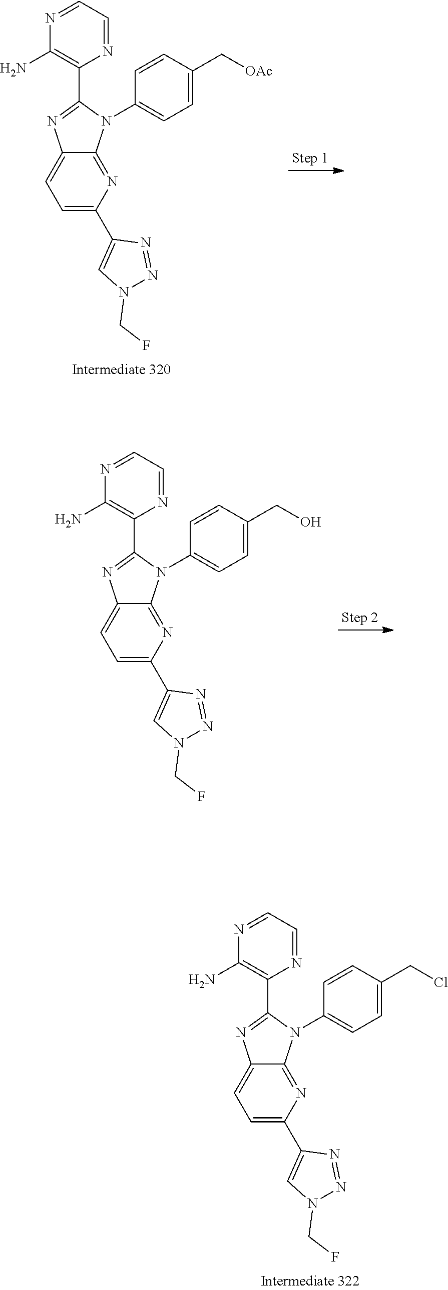 [SMILES: CC(=O)OCc1ccc(-n2c(-c3nccnc3N)nc3ccc(-c4cn(CF)nn4)nc32)cc1 ; Nc1nccnc1-c1nc2ccc(-c3cn(CF)nn3)nc2n1-c1ccc(CO)cc1 ; Nc1nccnc1-c1nc2ccc(-c3cn(CF)nn3)nc2n1-c1ccc(CCl)cc1]

### Step 1: (4-(2-(3-Aminopyrazin-2-yl)-5-(1-(fluoromethyl)-1H-1,2,3-triazol-4-yl)-3H-imidazo[4,5-b]pyridin-3-yl)phenyl)methanol

To a solution of Intermediate 320 (80 mg, 174 μmol) in MeOH (I mL), THF (I mL) and H₂O (0.5 mL) was added K₂CO₃ (72.2 mg, 522 mmol). The mixture was stirred at 25° C. for 0.5 hr. The residue was diluted with H₂O (5 mL) and extracted with CH₂Cl₂ (5 mL×3). The combined organic layers were washed with brine (3 mL×2), dried over Na₂SO₄, filtered and concentrated under reduced pressure. (4-(2-(3-Aminopyrazin-2-yl)-5-(1-(fluoromethyl)-1H-1,2,3-triazol-4-yl)-3H-imidazo[4,5-b]pyridin-3-yl)phenyl)methanol (80 mg, crude) was obtained as a yellow solid which was used into the next step directly. MS: m/z=417.9 [M+H]⁺. ¹H NMR (400 MHz, Dimethylsulfoxide-d₆) δ 8.75 (s, 1H), 8.38 (d, J=8.4 Hz, 1H), 8.16 (d, J=8.4 Hz, 1H), 8.01 (d, J=2.4 Hz, 1H), 7.75 (br s, 2H), 7.53 (d, J=2.4 Hz, 1H), 7.47-7.35 (m, 4H), 6.48 (d, J=50.8 Hz, 2H), 5.35 (br t, J=5.6 Hz, 1H), 4.61 (d, J=5.6 Hz, 2H). ¹⁹F NMR (400 MHz, Dimethylsulfoxide-d₆) δ −166.770.

### Step 2: 3-(3-(4-(Chloromethyl)phenyl)-5-(1-(fluoromethyl)-1H-1,2,3-triazol-4-yl)-3H-imidazo[4,5-b]pyridin-2-yl)pyrazin-2-amine

To a solution of (4-(2-(3-aminopyrazin-2-yl)-5-(1-(fluoromethyl)-1H-1,2,3-triazol-4-yl)-3H-imidazo[4,5-b]pyridin-3-yl)phenyl)methanol (80 mg, 192 μmol) in CH₂Cl₂ (3 mL) was added SOCl₂ (45.6 mg, 383 μmol). The reaction mixture was stirred at 40° C. for 1 hr. The reaction mixture was concentrated under reduced pressure to give 3-(3-(4-(chloromethyl)phenyl)-5-(1-(fluoromethyl)-1H-1,2,3-triazol-4-yl)-3H-imidazo[4,5-b]pyridin-2-yl)pyrazin-2-amine (Intermediate 322, 75 mg, crude, HCl salt) as a gray solid, which was used in the next step directly. MS: m/z=435.8, 437.8 [M+H]⁺.

### Intermediate 323: 3-(3-(4-(Chloromethyl)phenyl)-5-(2-ethyl-2H-1,2,3-triazol-4-yl)-3H-imidazo[4,5-b]pyridin-2-yl)pyrazin-2-amine

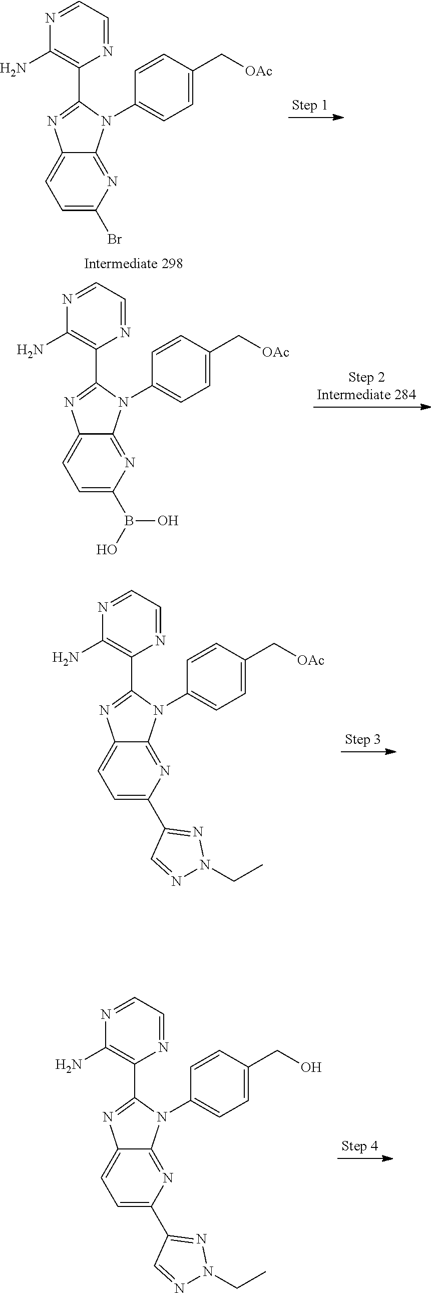

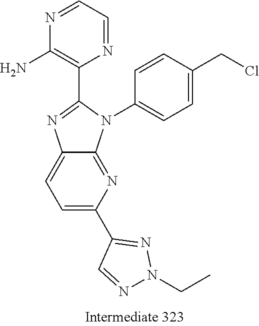 [SMILES: CCn1ncc(-c2ccc3nc(-c4nccnc4N)n(-c4ccc(CCl)cc4)c3n2)n1]

### Step 1: (3-(4-(Acetoxymethyl)phenyl)-2-(3-aminopyrazin-2-yl)-3H-imidazo[4,5-b]pyridin-5-yl)boronic acid

To a mixture of Intermediate 298 (380 mg, 865 μmol) and 4,4,4′,4′,5,5,5′,5′-octamethyl-2,2′-bi(1,3,2-dioxaborolane) (439 mg, 1.73 mmol) in 1,4-dioxane (8 mL) were added Pd(dppf)Cl₂ (63.3 mg, 86.5 μmol) and KOAc (255 mg, 2.60 mmol). The mixture was stirred at 85° C. for 1 hr. (3-(4-(Acetoxymethyl)phenyl)-2-(3-aminopyrazin-2-yl)-3H-imidazo[4,5-b]pyridin-5-yl)boronic acid was obtained as a black liquid, which was used in the next step without work-up and purification. MS: m/z=405.1 [M+H]⁺.

### Step 2: 4-(2-(3-Aminopyrazin-2-yl)-5-(2-ethyl-2H-1,2,3-triazol-4-yl)-3H-imidazo[4,5-b]pyridin-3-yl)benzyl acetate

To a mixture of (3-(4-(acetoxymethyl)phenyl)-2-(3-aminopyrazin-2-yl)-3H-imidazo[4,5-b]pyridin-5-yl)boronic acid (270 mg, 668 μmol) and Intermediate 284 (235 mg, 1.34 mmol) in 1,4-dioxane (10 mL) and H₂O (2 mL) were added Cs₂CO₃ (653 mg, 2.00 mmol) and Pd(dppf)Cl₂ (48.9 mg, 66.8 μmol). The mixture was stirred at 100° C. for 1 hr. The reaction mixture was quenched with H₂O (50 mL) and extracted with EtOAc (50 mL×2). The combined organic layers were washed with brine (50 mL), dried over anhydrous Na₂SO₄, filtered and concentrated. After purified by silica gel flash chromatography (EtOAc in petroleum ether=0-50%), 4-(2-(3-aminopyrazin-2-yl)-5-(2-ethyl-2H-1,2,3-triazol-4-yl)-3H-imidazo[4,5-b]pyridin-3-yl)benzyl acetate (250 mg, yield 82% for two steps) was obtained as a yellow solid. MS: m/z=456.2 [M+H]⁺. ¹H NMR (400 MHz, Dimethylsulfoxide-d₆) δ 8.35 (d, J=8.4 Hz, 1H), 8.05 (s, 1H), 8.02 (d, J=2.4 Hz, 1H), 7.98 (d, J=8.4 Hz, 1H), 7.77 (br s, 2H), 7.56-7.48 (m, 3H), 7.46-7.41 (m, 2H), 5.19 (s, 2H), 4.51 (q, J=7.2 Hz, 2H), 2.13 (s, 3H), 1.49 (t, J=7.2 Hz, 3H).

### Step 3: (4-(2-(3-Aminopyrazin-2-yl)-5-(2-ethyl-2H-1,2,3-triazol-4-yl)-3H-imidazo[4,5-b]pyridin-3-yl)phenyl)methanol

To a mixture of 4-(2-(3-aminopyrazin-2-yl)-5-(2-ethyl-2H-1,2,3-triazol-4-yl)-3H-imidazo[4,5-b]pyridin-3-yl)benzyl acetate (240 mg, 527 μmol) in THF (1 mL), MeOH (1 mL) and H₂O (0.5 mL) was added K₂CO₃ (218 mg, 1.58 mmol). The mixture was stirred at 20° C. for 0.5 hr. The reaction mixture was quenched with H₂O (50 mL) and extracted with EtOAc (30 mL×2). The combined organic layers were washed with brine (30 mL), dried over anhydrous Na₂SO₄, filtered and concentrated, (4-(2-(3-aminopyrazin-2-yl)-5-(2-ethyl-2H-1,2,3-triazol-4-yl)-3H-imidazo[4,5-b]pyridin-3-yl)phenyl)methanol (210 mg, yield: 96%) was obtained as a yellow solid. MS: m/z=414.2 [M+H]⁺. ¹H NMR (400 MHz, Dimethylsulfoxide-d₆) δ 8.34 (d, J=8.4 Hz, 1H), 8.03 (s, 1H), 8.01 (d, J=2.4 Hz, 1H), 7.97 (d, J=8.4 Hz, 1H), 7.74 (br s, 2H), 7.53 (d, J=2.4 Hz, 1H), 7.46-7.42 (m, 2H), 7.40-7.34 (m, 2H), 5.34 (t, J=5.6 Hz, 1H), 4.61 (d, J=6.0 Hz, 2H), 4.50 (q, J=7.2 Hz, 2H), 1.49 (t, J=7.2 Hz, 3H).

### Step 4: 3-(3-(4-(Chloromethyl)phenyl)-5-(2-ethyl-2H-1,2,3-triazol-4-yl)-3H-imidazo[4,5-b]pyridin-2-yl)pyrazin-2-amine

To a mixture of (4-(2-(3-aminopyrazin-2-yl)-5-(2-ethyl-2H-1,2,3-triazol-4-yl)-3H-imidazo[4,5-b]pyridin-3-yl)phenyl)methanol (200 mg, 484 μmol) in CH₂Cl₂ (0.5 mL) was added SOCl₂ (105 μL, 1.45 mmol). The mixture was stirred at 20° C. for 0.5 hr. The reaction was concentrated under reduced pressure. 3-(3-(4-(Chloromethyl)phenyl)-5-(2-ethyl-2H-1,2,3-triazol-4-yl)-3H-imidazo[4,5-b]pyridin-2-yl)pyrazin-2-amine (Intermediate 323, 209 mg) was obtained as a yellow solid. MS: m/z=432.0, 434.0 [M+H]⁺.

### Intermediate 324: 3-(3-(4-(Chloromethyl)phenyl)-5-(2-isopropyl-2H-1,2,3-triazol-4-yl)-3H-imidazo[4,5-b]pyridin-2-yl)pyrazin-2-amine

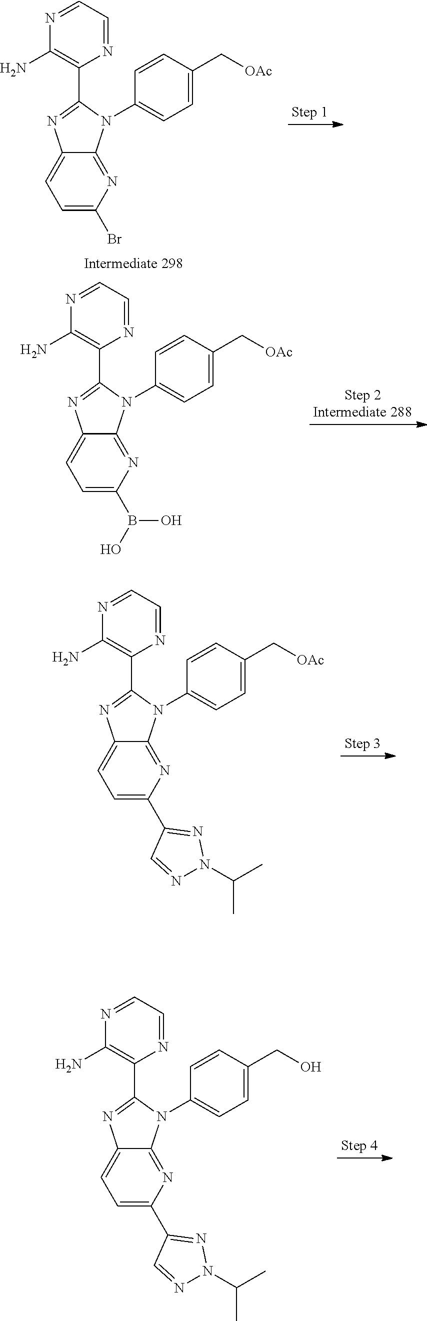

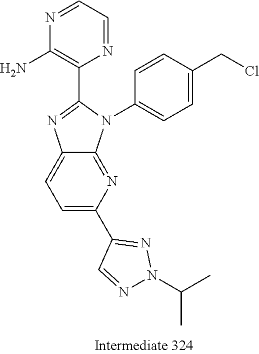 [SMILES: CC(C)n1ncc(-c2ccc3nc(-c4nccnc4N)n(-c4ccc(CCl)cc4)c3n2)n1]

### Step 1: (3-(4-(Acetoxymethyl)phenyl)-2-(3-aminopyrazin-2-yl)-3-imidazo[4,5-b]pyridin-5-yl)boronic acid

To a solution of Intermediate 298 (300 mg, 683 mol) in 1,4-dioxane (12.5 mL) were added 4,4,4′,4′,5,5,5,5′-octamethyl-2,2′-bi(1,3,2-dioxaborolane) (347 mg, 1.37 mmol), KOAc (201 mg, 2.05 mmol) and Pd(dppf)Cl₂ (50.0 mg, 68.3 mol) at 20′C. The reaction mixture was degassed and purged with N₂ three times and stirred at 80° C. for 2 hr under N₂. (3-(4-(Acetoxymethyl)phenyl)-2-(3-aminopyrazin-2-yl)-3H-imidazo[4,5-b]pyridin-5-yl)boronic acid was obtained as a black liquid, which was used in the next step without work-up and purification. MS: m/z=405.1 [M+H]⁺.

### Step 2: 4-(2-(3-Aminopyrazin-2-yl)-5-(2-isopropyl-211-1,2,3-triazol-4-yl)-3H-imidazo[4,5-b]pyridin-3-yl)benzyl acetate

To a solution of (3-(4-(acetoxymethyl)phenyl)-2-(3-aminopyrazin-2-yl)-311-imidazo[4,5-b]pyridin-5-yl)boronic acid (276 mg, 683 μmol) and Intermediate 288 (195 mg, 1.02 mmol) in 1,4-dioxane (12.5 mL) and H₂O (2.5 mL) were added Cs₂CO₃ (668 mg, 2.05 mmol) and Pd(dppf Cl₂ (50 mg, 68.3 μmol) at 20° C. The mixture was stirred at 100° C. for 1.5 hr under N₂. The mixture was diluted with H₂O (20 mL) and extracted with EtOAc (20 mL×2). The combined organic layers were washed with brine (20 mL), dried over anhydrous Na₂SO₄, filtered and concentrated. After purified by silica gel flash chromatography (EtOAc in petroleum ether=0-60%), 4-(2-(3-aminopyrazin-2-yl)-5-(2-isopropyl-2H-1,2,3-triazol-4-yl)-3H-imidazo[4,5-b]pyridin-3-yl)benzyl acetate (230 mg, 490 μmol, 72% yield for 2 steps) was obtained as a yellow solid. MS: m/z=470.2 [M+H]⁺. ¹H NMR (400 MHz, Dimethylsulfoxide-d₆) δ 8.35 (d, J=8.4 Hz, 1H), 8.05-7.95 (m, 3H), 7.77 (s, 2H), 7.54-7.48 (m, 3H), 7.46-7.41 (m, 2H), 5.19 (s, 2H), 4.93-4.84 (m, 1H), 2.11 (s, 3H), 1.53 (d, J=6.8 Hz, 6H).

### Step 3: (4-(2-(3-Aminopyrazin-2-yl)-5-(2-isopropyl-2H-1,2,3-triazol-4-yl)-3H-imidazo[4,5-b]pyridin-3-yl)phenyl)methanol

To a solution of 4-(2-(3-aminopyrazin-2-yl)-5-(2-isopropyl-2H-1,2,3-triazol-4-yl)-3H-imidazo[4,5-b]pyridin-3-yl)benzyl acetate (200 mg, 426 μmol) in THF (4 mL), MeOH (4 mL) and H₂O (2 mL) was added K₂CO₃ (177 mg, 1.28 mmol). The mixture was stirred at 20° C. for 0.5 hr. The mixture was diluted with H₂O (20 mL) and extracted with EtOAc (20 mL×2). The combined organic layers were washed with brine (20 mL), dried over anhydrous Na₂SO₄, filtered and concentrated. (4-(2-(3-Aminopyrazin-2-yl)-5-(2-isopropyl-2H-1,2,3-triazol-4-yl)-3H-imidazo[4,5-b]pyridin-3-yl)phenyl)methanol (180 mg, 421 μmol) was obtained as a yellow solid, which was used in the next step directly. MS: m/z=428.2 [M+H]⁺. ¹H NMR (400 MHz, Dimethylsulfoxide-d₆) 58.34 (d, J=8.4 Hz, 1H), 8.03-7.95 (m, 3H), 7.74 (s, 2H), 7.53 (d, J=2.4 Hz, 1H), 7.46-7.34 (m, 4H), 5.35 (t, J=6.0 Hz, 1H), 4.94-4.83 (m, 1H), 4.61 (d, J=5.6 Hz, 2H), 1.53 (d, J=6.8 Hz, 6H).

### Step 4: 3-(3-(4-(Chloromethyl)phenyl)-5-(2-isopropyl-2H-1,2,3-triazol-4-yl)-3H-imidazo[4,5-b]pyridin-2-yl)pyrazin-2-amine

To a solution of (4-(2-(3-aminopyrazin-2-yl)-5-(2-isopropyl-2H-1,2,3-triazol-4-yl)-3H-imidazo[4,5-b]pyridin-3-yl)phenyl)methanol (180 mg, 421 μmol) in CH₂Cl₂ (8 mL) was added SOCl₂ (150 mg, 1.26 mmol, 92.0 μL). The reaction mixture was stirred at 20° C. for 1 hr. The mixture was concentrated to give 3-(3-(4-(chloromethyl)phenyl)-5-(2-isopropyl-2H-1,2,3-triazol-4-yl)-3H-imidazo[4,5-b]pyridin-2-yl)pyrazin-2-amine (Intermediate 324, 187 mg, 419 μmol) as a yellow solid, which was used in the next step directly. MS: m/z=446.1, 448.1 [M+H]⁺. ¹H NMR (400 MHz, Dimethylsulfoxide-d₆) δ 8.35 (d, J=8.4 Hz, 1H), 8.04-8.01 (m, 2H), 7.99 (d, J=8.4 Hz, 1H), 7.57 (d, J=8.4 Hz, 2H), 7.52 (d, J=2.4 Hz, 1H), 7.44 (d, J=8.4 Hz, 2H), 4.93-4.84 (m, 3H), 1.53 (d, J=6.8 Hz, 6H).

### Intermediate 325: 3-(3-(4-(Chloromethyl)phenyl)-5-(2-cyclopropyl-2H-1,2,3-triazol-4-yl)-3H-imidazo[4,5-b]pyridin-2-yl)pyrazin-2-amine

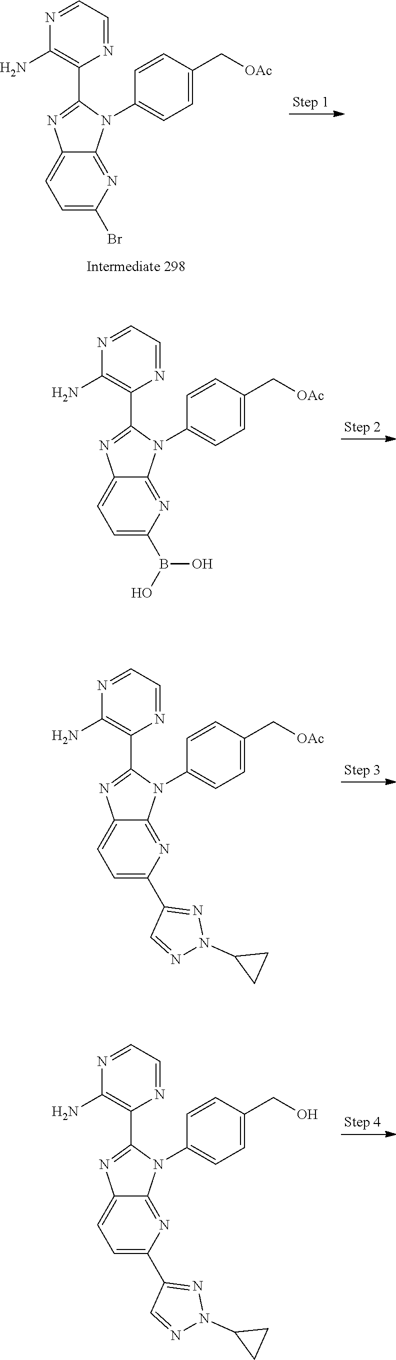 [SMILES: CC(=O)OCc1ccc(-n2c(-c3nccnc3N)nc3ccc(Br)nc32)cc1 ; CC(=O)OCc1ccc(-n2c(-c3nccnc3N)nc3ccc(B(O)O)nc32)cc1 ; CC(=O)OCc1ccc(-n2c(-c3nccnc3N)nc3ccc(-c4cnn(C5CC5)n4)nc32)cc1 ; Nc1nccnc1-c1nc2ccc(-c3cnn(C4CC4)n3)nc2n1-c1ccc(CO)cc1]

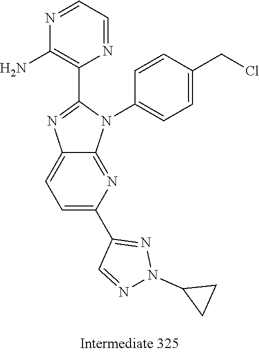 [SMILES: Nc1nccnc1-c1nc2ccc(-c3cnn(C4CC4)n3)nc2n1-c1ccc(CCl)cc1]

### Step 1: (3-(4-(Acetoxymethyl)phenyl)-2-(3-aminopyrazin-2-yl)-3H-imidazo[4,5-b]pyridin-5-yl)boronic acid

To a solution of Intermediate 298 (350 mg, 797 μmol) and 4,4,4′,4′,5,5,5′,5′-octamethyl-2,2′-bi(1,3,2-dioxaborolane) (405 mg, 1.59 mmol) in 1,4-dioxane (3 mL) were added Pd(dppf)Cl₂ (58.3 mg, 79.7 μmol) and KOAc (234.6 mg, 2.39 mmol). The reaction mixture was stirred at 85° C. for 2 hr under N₂ atmosphere. (3-(4-(Acetoxymethyl)phenyl)-2-(3-aminopyrazin-2-yl)-3H-imidazo[4,5-b]pyridin-5-yl)boronic acid (322 mg) was obtained as a black brown liquid, which was used in the next step without work-up and purification. MS: m/z=405.1 [M+H]⁺.

### Step 2: 4-(2-(3-Aminopyrazin-2-yl)-5-(2-cyclopropyl-2H-1,2,3-triazol-4-yl)-3H-imidazo[4,5-b]pyridin-3-yl)benzyl acetate

To a solution of (3-(4-(acetoxymethyl)phenyl)-2-(3-aminopyrazin-2-yl)-3H-imidazo[4,5-b]pyridin-5-yl)boronic acid (322 mg, 797 μmol) and 4-bromo-2-cyclopropyl-2H-1,2,3-triazole (180 mg, 956 μmol) in 1,4-dioxane (4 mL) and H₂O (0.8 mL) were added Cs₂CO₃ (779 mg, 2.39 mmol) and Pd(dppf)Cl₂ (58.3 mg, 79.7 μmol). The mixture was stirred at 100° C. for 2 hr under N₂ atmosphere. The mixture was diluted with H₂O (20 mL) and extracted with ethyl acetate (10 mL×2). The combined organic layers were dried over anhydrous Na₂SO₄, filtered and concentrated. After purified by silica gel flash chromatography (Eluent of 0%˜45% EtOAc in petroleum ether), 4-(2-(3-aminopyrazin-2-yl)-5-(2-cyclopropyl-2H-1,2,3-triazol-4-yl)-3H-imidazo[4,5-b]pyridin-3-yl)benzyl acetate (300 mg, yield: 81% for two steps) was obtained as a yellow solid. MS: m/z=468.2 [M+H]⁺. ¹H NMR (400 MHz, Dimethylsulfoxide-d₆) δ. 8.35 (d, J=8.4 Hz, 1H), 8.04-8.00 (m, 2H), 7.97 (d, J=8.0 Hz, 1H), 7.77 (br s, 2H), 7.53 (d, J=2.4 Hz, 1H), 7.50 (d, J=8.4 Hz, 2H), 7.43 (d, J=8.4 Hz, 2H), 5.19 (s, 2H), 4.26-4.19 (m, 1H), 2.13 (s, 3H), 1.29-1.22 (m, 2H), 1.16-1.09 (m, 2H).

### Step 3: (4-(2-(3-Aminopyrazin-2-yl)-5-(2-cyclopropyl-2H-1,2,3-triazol-4-yl)-3H-imidazo[4,5-b]pyridin-3-yl)phenyl)methanol

To a solution of 4-(2-(3-aminopyrazin-2-yl)-5-(2-cyclopropyl-2H-1,2,3-triazol-4-yl)-3H-imidazo[4,5-b]pyridin-3-yl)benzyl acetate (250 mg, 535 μmol) in THF (2 mL), H₂O (1 mL) and MeOH (2 mL) was added K₂CO₃ (222 mg, 1.60 mmol). The mixture was stirred at 25° C. for 1 hr. The reaction was concentrated, diluted with (5 mL) and extracted with ethyl acetate (5 mL×2). The combined organic layers were dried over anhydrous Na₂SO₄, filtered and concentrated. (4-(2-(3-Aminopyrazin-2-yl)-5-(2-cyclopropyl-2H-1,2,3-triazol-4-yl)-3H-imidazo[4,5-b]pyridin-3-yl)phenyl)methanol (228 mg) was obtained as a light yellow oil. MS: m/z=426.2 [M+H]⁺

### Step 4: 3-(3-(4-(Chloromethyl)phenyl)-5-(2-cyclopropyl-2H-1,2,3-triazol-4-yl)-3H-imidazo[4,5-b]pyridin-2-yl)pyrazin-2-amine

To a solution of (4-(2-(3-aminopyrazin-2-yl)-5-(2-cyclopropyl-2H-1,2,3-triazol-4-yl)-3H-imidazo[4,5-b]pyridin-3-yl)phenyl)methanol (228 mg, 535 μmol) in CH₂Cl₂ (1 mL) was added SOCl₂ (191 mg, 1.60 mmol). The mixture was stirred at 25° C. for 1 hr under N₂. The reaction was concentrated to give 3-(3-(4-(chloromethyl)phenyl)-5-(2-cyclopropyl-2H-1,2,3-triazol-4-yl)-3H-imidazo[4,5-b]pyridin-2-yl)pyrazin-2-amine (Intermediate 325, 237 mg, HCl salt) as a light yellow solid. MS: m/z=444.1, 446.1 [M+H]⁺.

### Intermediate 326 & 327: 2-(4-(2-(3-Aminopyrazin-2-yl)-3-(4-(chloromethyl)phenyl)-3H-imidazo[4,5-b]pyridin-5-yl)-2H-1,2,3-triazol-2-yl)acetonitrile and 2-(4-(2-(3-aminopyrazin-2-yl)-3-(4-(chloromethyl)phenyl)-3H-imidazo[4,5-b]pyridin-5-yl)-2H-1,2,3-triazol-2-yl)acetamide

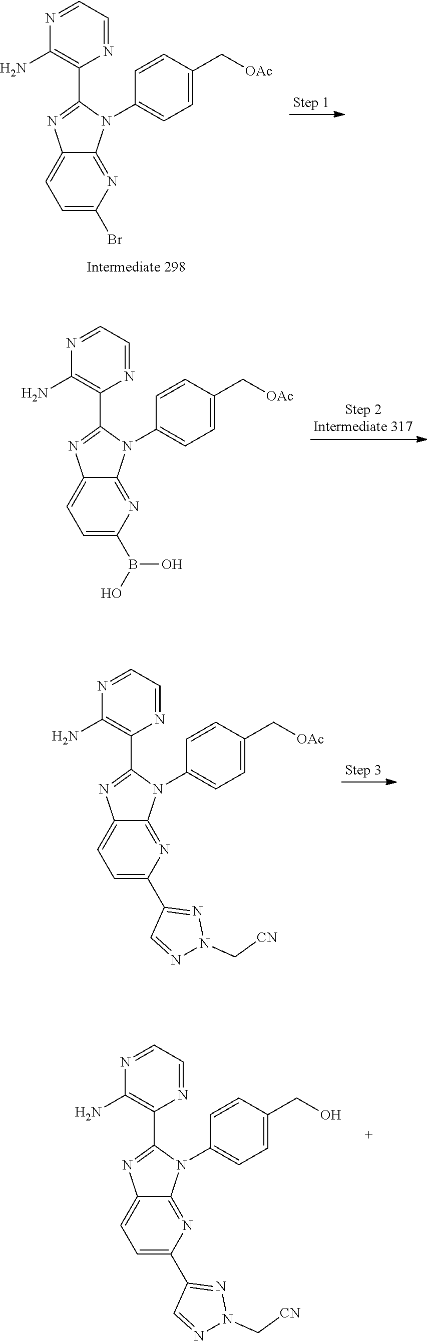 [SMILES: CC(=O)OCc1ccc(-n2c(-c3nccnc3N)nc3ccc(Br)nc32)cc1 ; CC(=O)OCc1ccc(-n2c(-c3nccnc3N)nc3ccc(B(O)O)nc32)cc1 ; CC(=O)OCc1ccc(-n2c(-c3nccnc3N)nc3ccc(-c4cnn(CC#N)n4)nc32)cc1 ; N#CCn1ncc(-c2ccc3nc(-c4nccnc4N)n(-c4ccc(CO)cc4)c3n2)n1]

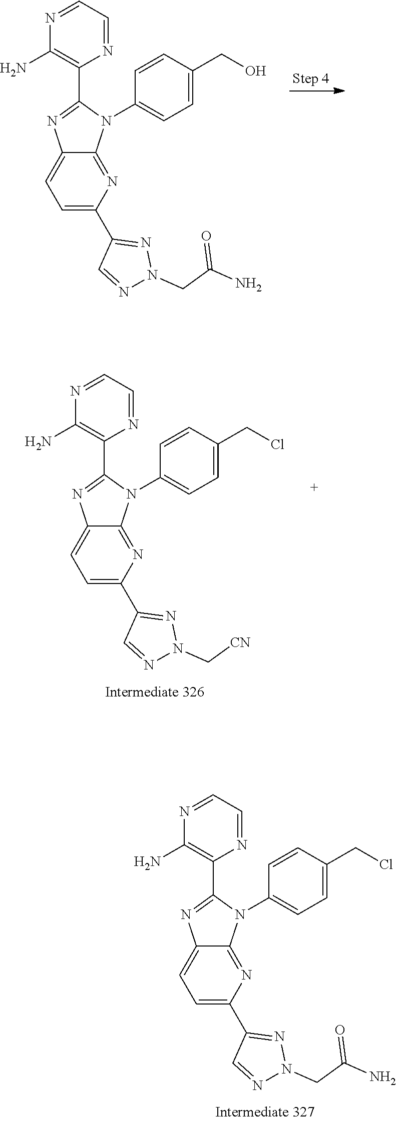 [SMILES: NC(=O)Cn1ncc(-c2ccc3nc(-c4nccnc4N)n(-c4ccc(CO)cc4)c3n2)n1 ; N#CCn1ncc(-c2ccc3nc(-c4nccnc4N)n(-c4ccc(CCl)cc4)c3n2)n1 ; NC(=O)Cn1ncc(-c2ccc3nc(-c4nccnc4N)n(-c4ccc(CCl)cc4)c3n2)n1]

### Step 1: (3-(4-(Acetoxymethyl)phenyl)-2-(3-aminopyrazin-2-yl)-3H-imidazo[4,5-b]pyridin-5-yl)boronic acid

To a solution of Intermediate 298 (360 mg, 820 μmol) and 4,4,4′,4′,5,5,5′,5′-octamethyl-2,2′-bi(1,3,2-dioxaborolane) (416 mg, 1.64 mmol) in 1,4-dioxane (2 mL) were added Pd(dppf)Cl₂ (60.0 mg, 82.0 μmol) and KOAc (241 mg, 2.46 mmol). The mixture was stirred at 85° C. for 2 hr under N₂ atmosphere. (3-(4-(Acetoxymethyl)phenyl)-2-(3-aminopyrazin-2-yl)-3H-imidazo[4,5-b]pyridin-5-yl)boronic acid was obtained as a black liquid, which was used in the next step without work-up and purification. MS: m/z=405.1 [M+H]⁺.

### Step 2: 4-(2-(3-Aminopyrazin-2-yl)-5-(2-(cyanomethyl)-2H-1,2,3-triazol-4-yl)-3H-imidazo[4,5-b]pyridin-3-yl)benzyl acetate

To a solution of (3-(4-(acetoxymethyl)phenyl)-2-(3-aminopyrazin-2-yl)-3H-imidazo[4,5-b]pyridin-5-yl)boronic acid (331 mg, 819 μmol) and Intermediate 317 (184 mg, 983 μmol) in 1,4-dioxane (4 mL) and H₂O (0.8 mL) were added Pd(dppf)Cl₂ (59.9 mg, 81.9 μmol) and Cs₂CO₃ (801 mg, 2.46 mmol). The mixture was stirred at 100° C. for 2 hr under N₂ atmosphere. The mixture was diluted with water (20 mL) and extracted with EtOAc (20 mL×2). The combined organic layers were dried over anhydrous Na₂SO₄, filtered and concentrated. After purified by silica gel flash chromatography (Eluent of 0%˜30% EtOAc in petroleum ether), 4-(2-(3-aminopyrazin-2-yl)-5-(2-(cyanomethyl)-2H-1,2,3-triazol-4-yl)-3H-imidazo[4,5-b]pyridin-3-yl)benzyl acetate (300 mg, yield: 79% for two steps) was obtained as a light yellow solid. MS: m/z=467.2 [M+H]⁺.

### Step 3: 2-(4-(2-(3-Aminopyrazin-2-yl)-3-(4-(hydroxymethyl)phenyl)-3H-imidazo[4,5-b]pyridin-5-yl)-2H-1,2,3-triazol-2-yl)acetonitrile & 2-(4-(2-(3-aminopyrazin-2-yl)-3-(4-(hydroxymethyl)phenyl)-3H-imidazo[4,5-b]pyridin-5-yl)-2H-1,2,3-triazol-2-yl)acetamide

To a solution of 4-(2-(3-aminopyrazin-2-yl)-5-(2-(cyanomethyl)-2H-1,2,3-triazol-4-yl)-3H-imidazo[4,5-b]pyridin-3-yl)benzyl acetate (200 mg, 429 μmol) in THF (2 mL) and H₂O (1 mL) was added LiOH (30.8 mg, 1.29 mmol). The mixture was stirred at 25° C. for 2 hr. The mixture was diluted with water (10 mL) and extracted with EtOAc (10 mL×2). The combined organic layers were dried over anhydrous Na₂SO₄, filtered and concentrated. The mixture (100 mg) of 2-(4-(2-(3-aminopyrazin-2-yl)-3-(4-(hydroxymethyl)phenyl)-3H-imidazo[4,5-b]pyridin-5-yl)-2H-1,2,3-triazol-2-yl)acetonitrile and 2-(4-(2-(3-aminopyrazin-2-yl)-3-(4-(hydroxymethyl)phenyl)-3H-imidazo[4,5-b]pyridin-5-yl)-2H-1,2,3-triazol-2-yl)acetamide was obtained as a light yellow oil. MS: m/z=447.0 [M+Na]⁺ & MS: m/z=443.1 [M+H]⁺.

### Step 4: 2-(4-(2-(3-Aminopyrazin-2-yl)-3-(4-(chloromethyl)phenyl)-3H-imidazo[4,5-b]pyridin-5-yl)-2H-1,2,3-triazol-2-yl)acetonitrile & 2-(4-(2-(3-aminopyrazin-2-yl)-3-(4-(chloromethyl)phenyl)-3H-imidazo[4,5-b]pyridin-5-yl)-2H-1,2,3-triazol-2-yl)acetamide

To a solution of 2-(4-(2-(3-aminopyrazin-2-yl)-3-(4-(hydroxymethyl)phenyl)-3H-imidazo[4,5-b]pyridin-5-yl)-2H-1,2,3-triazol-2-yl)acetonitrile (100 mg, 236 μmol, contained 2-(4-(2-(3-aminopyrazin-2-yl)-3-(4-(hydroxymethyl)phenyl)-3H-imidazo[4,5-b]pyridin-5-yl)-2H-1,2,3-triazol-2-yl)acetamide) in CH₂Cl₂ (1 mL) was added SOCl₂ (84.1 mg, 707 μmol). The mixture was stirred at 25° C. for 2 hr. The reaction was concentrated to give a mixture (104 mg, HCl salt, crude) of 2-(4-(2-(3-aminopyrazin-2-yl)-3-(4-(chloromethyl)phenyl)-3H-imidazo[4,5-b]pyridin-5-yl)-2H-1,2,3-triazol-2-yl)acetonitrile (Intermediate 326) and 2-(4-(2-(3-aminopyrazin-2-yl)-3-(4-(chloromethyl)phenyl)-3H-imidazo[4,5-b]pyridin-5-yl)-2H-1,2,3-triazol-2-yl)acetamide (Intermediate 327) as a light yellow solid, which was used in the next step without purification. MS: m/z=443.1, 445.1 [M+H]⁺. MS: m/z=483.0, 485.0 [M+Na]⁺.

### Intermediate 328: 3-(3-(4-(Chloromethyl)phenyl)-5-(5-(trifluoromethyl)pyridin-2-yl)-3H-imidazo[4,5-b]pyridin-2-yl)pyridin-2-amine

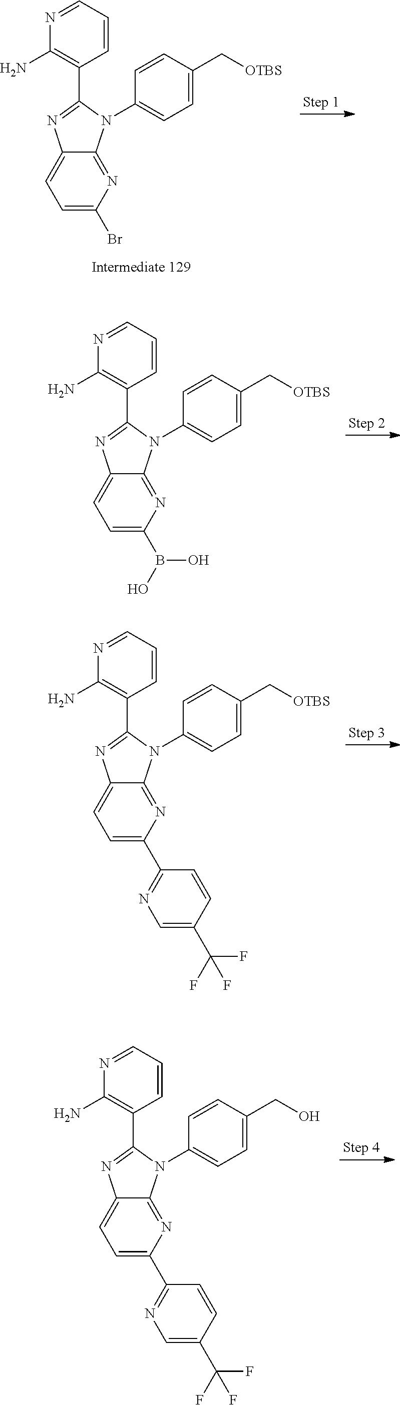

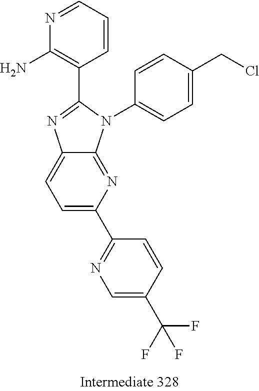 [SMILES: Nc1ncccc1-c1nc2ccc(-c3ccc(C(F)(F)F)cn3)nc2n1-c1ccc(CCl)cc1]

### Step 1; (2-(2-Aminopyridin-3-yl)-3-(4-(((tert-butyldimethylsilyl)oxy)methyl)phenyl)-3H-imidazo[4,5-b]pyridin-5-yl)boronic acid

A mixture of Intermediate 129 (500 mg, 979 μmol), 4,4,4′,4′,5,5,5′,5′-octamethyl-2,2′-bi(1,3,2-dioxaborolane) (498 mg, 1.96 mmol), KOAc (288 mg, 2.94 mmol) and Pd(dppf)Cl₂ (71.7 mg, 97.9 μmol) in 1,4-dioxane (5 mL) was degassed, purged with N₂ three times, and stirred at 90° C. for 8 hr under N₂ atmosphere. (2-(2-Aminopyridin-3-yl)-3-(4-(((tert-butyldimethylsilyl)oxy)methyl)phenyl)-3H-imidazo[4,5-b]pyridin-5-yl)boronic acid was obtained as a black liquid, which was used in the next step without work-up and purification. MS: m/z=476.2 [M+H]⁺.

### Step 2: 3-(3-(4-(((tert-Butyldimethylsilyl)oxy)methyl)phenyl)-5-(5-(trifluoromethyl)pyridin-2-yl)-3H-imidazo[4,5-b]pyridin-2-yl)pyridin-2-amine

A mixture of (2-(2-aminopyridin-3-yl)-3-(4-(((tert-butyldimethylsilyl)oxy)methyl)phenyl)-3H-imidazo[4,5-b]pyridin-5-yl)boronic acid (465 mg, 978 μmol), 2-bromo-5-(trifluoromethyl)pyridine (221 mg, 978 μmol), Pd(dppf)Cl₂ (71.6 mg, 98 μmol) and Cs₂CO₃ (957 mg, 2.93 mmol) in 1,4-dioxane (10 mL) and H₂O (2 mL) was degassed, purged with N₂ three times, and stirred at 80° C. for 16 hr under N₂ atmosphere. The reaction mixture was quenched with H₂O (20 mL) and extracted with EtOAc (20 mL×3). The combined organic layers were washed with brine (60 mL), dried over Na₂SO₄, filtered and concentrated under reduced pressure. After purified by silica gel flash chromatography (Eluent of 15%˜30% EtOAc in petroleum ether), 3-(3-(4-(((tert-butyldimethylsilyl)oxy)methyl)phenyl)-5-(5-(trifluoromethyl)pyridin-2-yl)-3H-imidazo[4,5-b]pyridin-2-yl)pyridin-2-amine (340 mg, yield: 54% for two steps) was obtained as a brown solid. MS: m/z=577.2 [M+H]⁺. ¹H NMR (400 MHz, Dimethylsulfoxide-d₆) δ 9.06 (s, 1H), 8.53 (d, J=8.4 Hz, 1H), 8.42-8.25 (m, 3H), 8.01 (dd, J=4.8, 2.0 Hz, 1H), 7.57-7.43 (m, 4H), 7.22 (dd, J=7.6, 1.6 Hz, 1H), 7.02 (br s, 2H), 6.42-6.36 (m, 1H), 4.84 (s, 2H), 0.93 (s, 9H), 0.12 (s, 6H). ¹⁹F NMR (400 MHz, Dimethylsulfoxide-d₆) δ −60.75.

### Step 3: (4-(2-(2-Aminopyridin-3-yl)-5-(5-(trifluoromethyl)pyridin-2-yl)-3H-imidazo[4,5-b]pyridin-3-yl)phenyl)methanol

To a solution of 3-(3-(4-(((tert-butyldimethylsilyl)oxy)methyl)phenyl)-5-(5-(trifluoromethyl)pyridin-2-yl)-3H-imidazo[4,5-b]pyridin-2-yl)pyridin-2-amine (340 mg, 590 μmol) in THF (5 mL) was added TBAF (1.8 mL, 1 M in THF). The mixture was stirred at 25° C. for 1 hr. The reaction mixture was quenched with H₂O (20 mL) at 25° C. and extracted with EtOAc (20 mL×3). The combined organic layers were washed with brine (20 mL×3), dried over Na₂SO₄, filtered and concentrated under reduced pressure. The crude product was triturated with CH₂Cl₂:petroleum ether=1:10 (6 mL) at 25° C. for 10 min. (4-(2-(2-Aminopyridin-3-yl)-5-(5-(trifluoromethyl)pyridin-2-yl)-3H-imidazo[4,5-b]pyridin-3-yl)phenyl)methanol (220 mg, yield: 75%) was obtained as a brown solid. MS: m/z=463.1 [M+H]⁺. ¹H NMR (400 MHz, Dimethylsulfoxide-d₆) δ 9.06 (s, 1H), 8.53 (d, J=8.4 Hz, 1H), 8.39-8.26 (m, 3H), 8.01 (dd, J=4.8, 2.0 Hz, 1H), 7.54-7.45 (m, 4H), 7.25 (dd, J=7.6, 1.6 Hz, 1H), 6.95 (br s, 2H), 6.43 (dd, J=7.6, 4.8 Hz, 1H), 5.39 (br t, J=5.6 Hz, 1H), 4.62 (d, J=5.6 Hz, 2H). ¹⁹F NMR (400 MHz, Dimethylsulfoxide-d₆) δ −60.757.

### Step 4: 3-(3-(4-(Chloromethyl)phenyl)-5-(5-(trifluoromethyl)pyridin-2-yl)-3H-imidazo[4,5-b]pyridin-2-yl)pyridin-2-amine

To a solution of (4-(2-(2-aminopyridin-3-yl)-5-(5-(trifluoromethyl)pyridin-2-yl)-3H-imidazo[4,5-b]pyridin-3-yl)phenyl)methanol (220 mg, 476 μmol) in CH₂Cl₂ (5 mL) was added SOCl₂ (104 μL, 1.43 mmol). The mixture was stirred at 40° C. for 0.5 hr. The reaction was concentrated under reduced pressure to give 3-(3-(4-(chloromethyl)phenyl)-5-(5-(trifluoromethyl)pyridin-2-yl)-3H-imidazo[4,5-b]pyridin-2-yl)pyridin-2-amine (Intermediate 328, 246 mg, HCl salt) as a brown solid. MS: m/z=481.1, 483.1 [M+H]⁺.

### Intermediate 329: 3-(3-(4-(Chloromethyl)phenyl)-5-(2-methyl-2H-1,2,3-triazol-4-yl-5-d)-3H-imidazo[4,5-b]pyridin-2-yl)pyrazin-2-amine

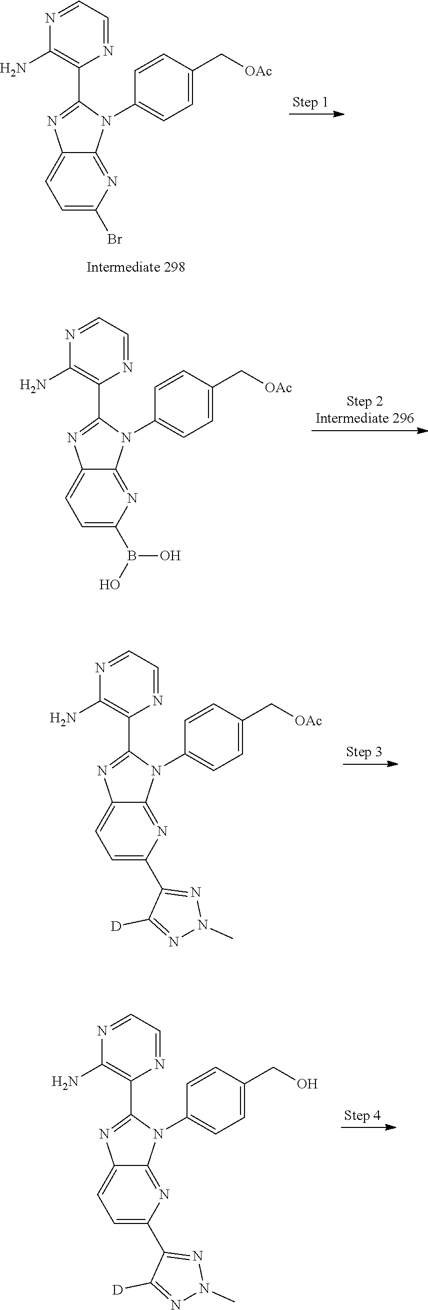 [SMILES: CC(=O)OCc1ccc(-n2c(-c3nccnc3N)nc3ccc(Br)nc32)cc1 ; CC(=O)OCc1ccc(-n2c(-c3nccnc3N)nc3ccc(B(O)O)nc32)cc1 ; [2H]c1nn(C)nc1-c1ccc2nc(-c3nccnc3N)n(-c3ccc(COC(C)=O)cc3)c2n1 ; [2H]c1nn(C)nc1-c1ccc2nc(-c3nccnc3N)n(-c3ccc(CO)cc3)c2n1]

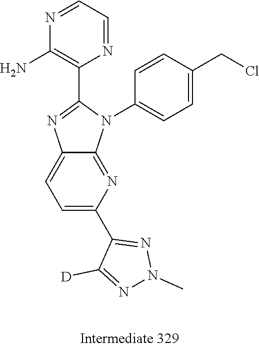 [SMILES: [2H]c1nn(C)nc1-c1ccc2nc(-c3nccnc3N)n(-c3ccc(CCl)cc3)c2n1]

### Step 1: (3-(4-(Acetoxymethyl)phenyl)-2-(3-aminopyrazin-2-yl)-3H-imidazo[4,5-b]pyridin-5-yl)boronic acid

A mixture of Intermediate 298 (500 mg, 1.14 mmol), 4,4,4′,4′,5,5,5′,5′-octamethyl-2,2′-bi(1,3,2-dioxaborolane) (578 mg, 2.28 mmol), Pd(dppf)Cl₂ (83.3 mg, 114 μmol) and KOAc (335 mg, 3.41 mmol) in 1,4-dioxane (5 mL) was degassed and purged with N₂ three times and was stirred at 90° C. for 2 hr under N₂ atmosphere. (3-(4-(Acetoxymethyl)phenyl)-2-(3-aminopyrazin-2-yl)-3H-imidazo[4,5-b]pyridin-5-yl)boronic acid was obtained as a black liquid, which was used in the next step without work-up and purification. MS: m/z=405.0 [M+H]⁺.

### Step 2: 4-(2-(3-Aminopyrazin-2-yl)-5-(2-methyl-2H-1,2,3-triazol-4-yl-5-d)-3H-imidazo[4,5-b]pyridin-3-yl)benzyl acetate

A mixture of (3-(4-(acetoxymethyl)phenyl)-2-(3-aminopyrazin-2-yl)-3H-imidazo[4,5-b]pyridin-5-yl)boronic acid (460 mg, 1.14 mmol), Intermediate 296 (371 mg, 2.28 mmol), Cs₂CO₃ (1.11 g, 3.41 mmol) and Pd(dppf)Cl₂ (83.3 mg, 114 μmol) in 1,4-dioxane (6 mL) and H₂O (1.5 mL) was degassed, purged with N₂ three times and stirred at 90° C. for 1 hr under N₂ atmosphere. The reaction mixture was quenched with H₂O (5 mL) and extracted with CH₂Cl₂ (15 mL×2). The combined organic layers were washed with brine (50 mL), dried over Na₂SO₄, filtered and concentrated under reduced pressure. After purified by silica gel flash chromatography (Eluent of 5%˜45% EtOAc in petroleum ether), 4-(2-(3-aminopyrazin-2-yl)-5-(2-methyl-2H-1,2,3-triazol-4-yl-5-d)-3H-imidazo[4,5-b]pyridin-3-yl)benzyl acetate (380 mg, yield: 72% for two steps) was obtained as a yellow solid. MS: m/z=465.1 [M+Na]⁺. D %: 1D %=94.1%. ¹H NMR (400 MHz, Dimethylsulfoxide-d₆) δ 8.35 (d, J=8.4 Hz, 1H), 8.02 (d, J=2.4 Hz, 1H), 7.97 (d, J=8.4 Hz, 1H), 7.77 (br s, 2H), 7.54-7.49 (m, 3H), 7.46-7.41 (m, 2H), 5.19 (s, 2H), 4.22 (s, 3H), 2.13 (s, 3H).

### Step 3: (4-(2-(3-Aminopyrazin-2-yl)-5-(2-methyl-2H-1,2,3-triazol-4-yl-5-d)-3H-imidazo[4,5-b]pyridin-3-yl)phenyl)methanol

To a solution of 4-(2-(3-aminopyrazin-2-yl)-5-(2-methyl-2H-1,2,3-triazol-4-yl-5-4)-3H-imidazo[4,5-b]pyridin-3-yl)benzyl acetate (380 mg, 859 μmol) in THF (10 mL), MeOH (10 mL) and H₂O (10 mL) was added K₂CO₃ (593 mg, 4.29 mmol). The mixture was stirred at 25° C. for 1 hr. The reaction mixture was quenched with H₂O (20 mL) at 25° C. and extracted with CH₂Cl₂ (25 mL×2). The combined organic layers were washed with brine (30 mL), dried over Na₂SO₄, filtered and concentrated under reduced pressure. (4-(2-(3-Aminopyrazin-2-yl)-5-(2-methyl-2H-1,2,3-triazol-4-yl-5-d)-3H-imidazo[4,5-b]pyridin-3-yl)phenyl)methanol (340 mg) was obtained as a yellow solid. MS: m/z=401.0 [M+H]⁺. ¹H NMR (400 MHz, Dimethylsulfoxide-d₆) δ 8.34 (d, J=8.4 Hz, 1H), 8.05-7.94 (m, 2H), 7.74 (br s, 2H), 7.54 (d, J=2.4 Hz, 1H), 7.46-7.41 (m, 2H), 7.39-7.34 (m, 2H), 5.33 (br t, J=5.6 Hz, 1H), 4.61 (d, J=6.0 Hz, 2H), 4.21 (s, 3H).

### Step 4: 3-(3-(4-(Chloromethyl)phenyl)-5-(2-methyl-2H-1,2,3-triazol-4-yl-5-d)-3H-imidazo[4,5-b]pyridin-2-yl)pyrazin-2-amine

To a solution of (4-(2-(3-aminopyrazin-2-yl)-5-(2-methyl-2H-1,2,3-triazol-4-yl-5-d)-3H-imidazo[4,5-b]pyridin-3-yl)phenyl)methanol (330 mg, 824 μmol) in CH₂Cl₂ (10 mL) was added SOCl₂ (490 mg, 4.12 mmol). The mixture was stirred at 25° C. for 1 hr. The reaction was concentrated under reduced pressure to give 3-(3-(4-(chloromethyl)phenyl)-5-(2-methyl-2H-1,2,3-triazol-4-yl-5-d)-3H-imidazo[4,5-b]pyridin-2-yl)pyrazin-2-amine (Intermediate 329, 375 mg, HCl salt) as a yellow solid. MS: m/z=419.1, 421.1 [M+H]⁺. ¹H NMR (400 MHz, Methanol-dr) 68.33 (d, J=8.8 Hz, 1H), 8.11 (d, J=8.4 Hz, 1H), 7.89 (d, J=3.2 Hz, 1H), 7.71 (d, J=2.8 Hz, 1H), 7.64-7.60 (m, 2H), 7.48-7.43 (m, 2H), 4.80 (s, 2H), 4.22 (s, 3H).

### Intermediate 330: 3-(3-(4-(Chloromethyl)phenyl)-5-(2,5-dimethyl-2H-1,2,3-triazol-4-yl)-3H-imidazo[4,5-b]pyridin-2-yl)pyrazin-2-amine

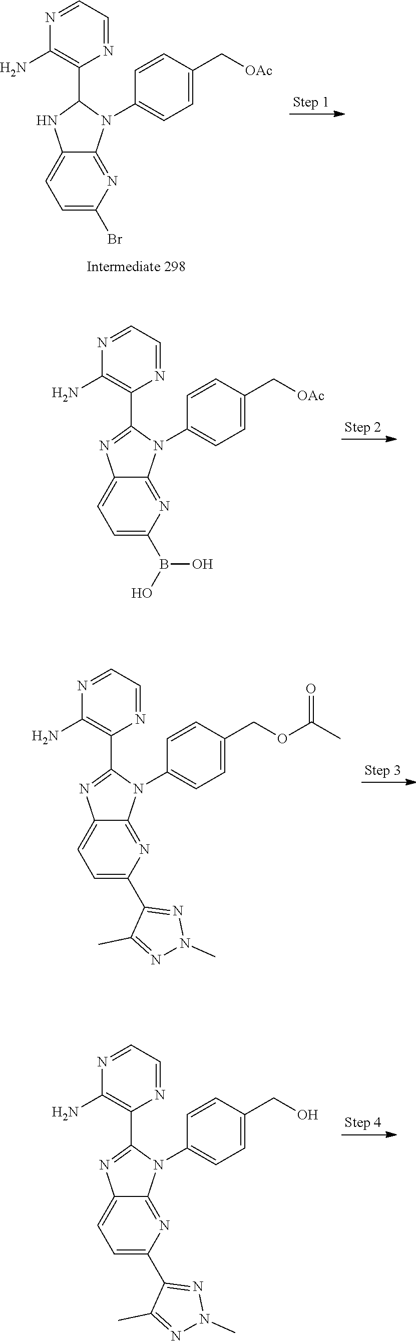 [SMILES: CC(=O)OCc1ccc(N2c3nc(Br)ccc3NC2c2nccnc2N)cc1 ; CC(=O)OCc1ccc(-n2c(-c3nccnc3N)nc3ccc(B(O)O)nc32)cc1 ; CC(=O)OCc1ccc(-n2c(-c3nccnc3N)nc3ccc(-c4nn(C)nc4C)nc32)cc1 ; Cc1nn(C)nc1-c1ccc2nc(-c3nccnc3N)n(-c3ccc(CO)cc3)c2n1]

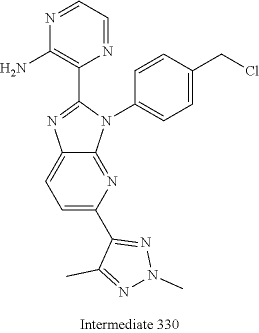 [SMILES: Cc1nn(C)nc1-c1ccc2nc(-c3nccnc3N)n(-c3ccc(CCl)cc3)c2n1]

### Step 1: (3-(4-(Acetoxymethyl)phenyl)-2-(3-aminopyrazin-2-yl)-3H-imidazo[4,5-b]pyridin-5-yl)boronic acid

To a mixture of Intermediate 298 (350 mg, 797 μmol), 4,4,4′,4′,5,5,5′,5′-octamethyl-2,2′-bi(1,3,2-dioxaborolane) (405 mg, 1.59 mmol), Pd(dppf)Cl₂ (58.3 mg, 79.7 μmol) and KOAc (235 mg, 2.39 mmol) in 1,4-dioxane (0.8 mL) was degassed and purged with N₂ three times. The mixture was stirred at 85° C. for 1 hr under N₂ atmosphere. (3-(4-(Acetoxymethyl)phenyl)-2-(3-aminopyrazin-2-yl)-3H-imidazo[4,5-b]pyridin-5-yl)boronic acid was obtained as a black liquid, which was used in the next step directly. MS: m/z=405.1 [M+H]⁺.

### Step 2: 4-(2-(3-Aminopyrazin-2-yl)-5-(2,5-dimethyl-2H-1,2,3-triazol-4-yl)-3H-imidazo[4,5-b]pyridin-3-yl)benzyl acetate

To a mixture of (3-(4-(acetoxymethyl)phenyl)-2-(3-aminopyrazin-2-yl)-3H-imidazo[4,5-b]pyridin-5-yl)boronic acid (322 mg, 797 μmol), 4-bromo-2,5-dimethyl-2H-1,2,3-triazole (280 mg, 1.59 mmol), Cs₂CO₃ (779 mg, 2.39 mmol) and Pd(dppf)Cl₂ (58.3 mg, 79.7 μmol) in 1,4-dioxane (0.8 mL) and H₂O (0.2 mL) was degassed and purged with N₂ for 3 times, the mixture was stirred at 100° C. for 1 hr under N₂. The mixture was diluted with H₂O (50 mL) and extracted with EtOAc (30 mL×2). The combined organic layers were washed with brine (20 mL), dried over anhydrous Na₂SO₄, filtered and concentrated. After purified by silica gel flash chromatography (Eluent of 0-44% EtOAc in Petroleum ether), 4-(2-(3-aminopyrazin-2-yl)-5-(2,5-dimethyl-2H-1,2,3-triazol-4-yl)-3H-imidazo[4,5-b]pyridin-3-yl)benzyl acetate (251 mg, yield: 69% for two steps) was obtained as a light yellow solid. MS: m/z=456.2 [M+H]⁺. ¹H NMR (400 MHz, Dimethylsulfoxide-d₆) 8.32 (d, J=8.4 Hz, 1H), 8.03 (d, J=2.4 Hz, 1H), 7.94 (d, J=8.4 Hz, 1H), 7.82 (br s, 2H), 7.55-7.52 (m, 2H), 7.49-7.45 (m, 4H), 5.18 (s, 2H), 4.13 (s, 3H), 2.34 (s, 3H),

### Step 3: (4-(2-(3-Aminopyrazin-2-yl)-5-(2,5-dimethyl-2H-1,2,3-triazol-4-yl)-3H-imidazo[4,5-b]pyridin-3-yl)phenyl)methanol

To a solution of 4-(2-(3-aminopyrazin-2-yl)-5-(2,5-dimethyl-2H-1,2,3-triazol-4-yl)-3H-imidazo[4,5-b]pyridin-3-yl)benzyl acetate (155 mg, 340 μmol,) in MeOH (1.5 mL), THF (1.5 mL), H₂O (0.75 mL) was added K₂CO₃ (141 mg, 1.02 mmol). The mixture was stirred at 25° C. for 2 hr under N₂. The mixture was diluted with H₂O (50 mL) and extracted with EtOAc (30 mL×3). The combined organic layers were washed with brine (30 mL), dried over anhydrous Na₂SO₄, filtered and concentrated. (4-(2-(3-Aminopyrazin-2-yl)-5-(2,5-dimethyl-2H-1,2,3-triazol-4-yl)-3H-imidazo[4,5-b]pyridin-3-yl)phenyl)methanol (135 mg) was obtained as a brown solid, which was used in the next step without further purification. MS: m/z=414.2 [M+H]⁺. ¹H NMR (400 MHz, Dimethylsulfoxide-d₆) 8.31 (d, J=8.8 Hz, 1H), 8.02 (d, J=2.4 Hz, 1H), 7.94 (d, J=8.4 Hz, 1H), 7.76 (br s, 2H), 7.56 (d, J=2.4 Hz, 1H), 7.43-7.40 (m, 2H), 7.39-7.36 (m, 2H), 5.34 (t, J=5.6 Hz, 1H), 4.60 (d, J=5.6 Hz, 2H), 4.13 (s, 3H), 2.36 (s, 3H).

### Step 4: 3-(3-(4-(Chloromethyl)phenyl)-5-(2,5-dimethyl-2H-1,2,3-triazol-4-yl)-3H-imidazo[4,5-b]pyridin-2-yl)pyrazin-2-amine

To a solution of (4-(2-(3-aminopyrazin-2-yl)-5-(2,5-dimethyl-2H-1,2,3-triazol-4-yl)-3H-imidazo[4,5-b]pyridin-3-yl)phenyl)methanol (125 mg, 302 μmol) in CH₂Cl₂ (2.5 mL) was added SOCl₂ (108 mg, 907 μmol). The mixture was stirred at 25° C. for 2 hr under N₂. The reaction mixture was concentrated, 3-(3-(4-(chloromethyl)phenyl)-5-(2,5-dimethyl-2H-1,2,3-triazol-4-yl)-3H-imidazo[4,5-b]pyridin-2-yl)pyrazin-2-amine (Intermediate 330, 110 mg) was obtained as a yellow solid, which was used in the next step without further purification. MS: m/z=432.1, 434.1[M+H]⁺.

### Intermediate 331: 3-(3-(4-(Chloromethyl)phenyl)-5-(6-methoxypyridin-3-yl)-3H-imidazo[4,5-b]pyridin-2-yl)pyrazin-2-amine

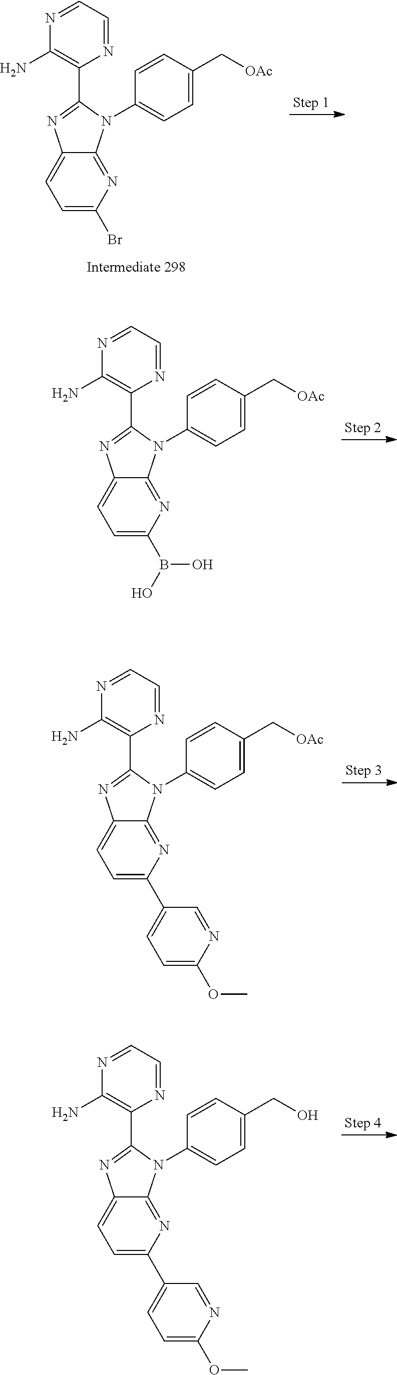 [SMILES: CC(=O)OCc1ccc(-n2c(-c3nccnc3N)nc3ccc(Br)nc32)cc1 ; CC(=O)OCc1ccc(-n2c(-c3nccnc3N)nc3ccc(B(O)O)nc32)cc1 ; COc1ccc(-c2ccc3nc(-c4nccnc4N)n(-c4ccc(COC(C)=O)cc4)c3n2)cn1 ; COc1ccc(-c2ccc3nc(-c4nccnc4N)n(-c4ccc(CO)cc4)c3n2)cn1]

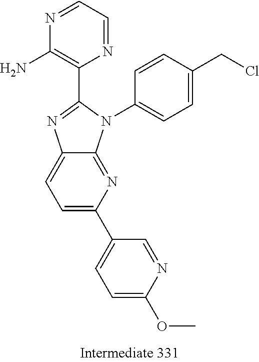 [SMILES: COc1ccc(-c2ccc3nc(-c4nccnc4N)n(-c4ccc(CCl)cc4)c3n2)cn1]

### Step 1: (3-(4-(Acetoxymethyl)phenyl)-2-(3-aminopyrazin-2-yl)-3H-imidazo[4,5-b]pyridin-5-yl)boronic acid

A mixture of Intermediate 298 (250 mg, 569 μmol), 4,4,4′,4′,5,5,5′,5′-octamethyl-2,2′-bi(1,3,2-dioxaborolane) (289 mg, 1.14 mmol), Pd(dppf)Cl₂ (41.6 mg, 56.9 μmol) and KOAc (168 mg, 1.71 mmol) in 1,4-dioxane (3 mL) was degassed, purged with N₂ three times and stirred at 90° C. for 2 hr under N₂ atmosphere. (3-(4-(Acetoxymethyl)phenyl)-2-(3-aminopyrazin-2-yl)-3H-imidazo[4,5-b]pyridin-5-yl)boronic acid was obtained as a black liquid, which was used in the next step without work-up and purification. MS: m/z=405.1 [M+H]⁺.

### Step 2: 4-(2-(3-Aminopyrazin-2-yl)-5-(6-methoxypyridin-3-yl)-3H-imidazo[4,5-b]pyridin-3-yl)benzyl acetate

A mixture of (3-(4-(acetoxymethyl)phenyl)-2-(3-aminopyrazin-2-yl)-3H-imidazo[4,5-b]pyridin-5-yl)boronic acid (230 mg, 569 μmol), 5-bromo-2-methoxypyridine (128 mg, 683 μmol), Cs₂CO₃ (556 mg, 1.71 mmol) and Pd(dppf)Cl₂ (41.6 mg, 56.9 μmol) in 1,4-dioxane (6 mL) and H₂O (1.2 mL) was degassed, purged with N₂ three times and stirred at 90° C. for 1 hr under N₂ atmosphere. The reaction mixture was quenched with H₂O (5 mL) and extracted with CH₂Cl₂ (15 mL×2). The combined organic layers were washed with brine (50 mL), dried over Na₂SO₄, filtered, and concentrated under reduced pressure. After purified by silica gel flash chromatography (Eluent of 10%˜50% EtOAc in petroleum ether), 4-(2-(3-aminopyrazin-2-yl)-5-(6-methoxypyridin-3-yl)-3H-imidazo[4,5-b]pyridin-3-yl)benzyl acetate (195 mg, yield: 66% for two steps) was obtained as a yellow solid. MS: m/z=490.2 [M+Na]^{+ 1}H NMR (400 MHz, Chloroform-d) δ 8.83 (d, J=2.4 Hz, 1H), 8.20 (dd, J=8.4, 2.4 Hz, 1H), 8.15 (d, J=8.4 Hz, 1H), 7.96 (d, J=2.4 Hz, 1H), 7.74 (d, J=8.4 Hz, 1H), 7.60 (d, J=2.4 Hz, 1H), 7.49-7.45 (m, 2H), 7.40-7.36 (m, 2H), 7.19-6.93 (m, 2H), 6.80 (d, J=8.8 Hz, 1H), 5.24 (s, 2H), 3.98 (s, 3H), 2.18 (s, 3H).

### Step 3: (4-(2-(3-Aminopyrazin-2-yl)-5-(6-methoxypyridin-3-yl)-3H-imidazo[4,5-b]pyridin-3-yl)phenyl)methanol

To a solution of 4-(2-(3-aminopyrazin-2-yl)-5-(6-methoxypyridin-3-yl)-3H-imidazo[4,5-b]pyridin-3-yl)benzyl acetate (195 mg, 417 μmol) in THF (5 mL), MeOH (5 mL) and H₂O (5 mL) was added K₂CO₃ (173 mg, 1.25 mmol). The mixture was stirred at 25° C. for 1 hr. The reaction mixture was quenched with H₂O (20 mL) at 25° C. and extracted with CH₂Cl₂ (25 mL×2). The combined organic layers were washed with brine (30 mL), dried over Na₂SO₄, filtered and concentrated under reduced pressure. (4-(2-(3-Aminopyrazin-2-yl)-5-(6-methoxypyridin-3-yl)-3H-imidazo[4,5-b]pyridin-3-yl)phenyl)methanol (177 mg) was obtained as a yellow solid. MS: m/z=426.2 [M+H]⁺. ¹H NMR (400 MHz, Dimethylsulfoxide-d₆) δ 8.84 (d, J=2.0 Hz, 1H), 8.36-8.26 (m, 2H), 8.07-7.98 (m, 2H), 7.80 (br s, 2H), 7.54 (d, J=2.4 Hz, 1H), 7.49-7.42 (m, 2H), 7.41-7.34 (m, 2H), 6.92 (d, J=8.4 Hz, 1H), 5.33 (br t, J=5.2 Hz, 1H), 4.61 (d, J=5.2 Hz, 2H), 3.89 (s, 3H).

### Step 4: 3-(3-(4-(Chloromethyl)phenyl)-5-(6-methoxypyridin-3-yl)-3H-imidazo[4,5-b]pyridin-2-yl)pyrazin-2-amine

To a solution of (4-(2-(3-aminopyrazin-2-yl)-5-(6-methoxypyridin-3-yl)-3H-imidazo[4,5-b]pyridin-3-yl)phenyl)methanol (175 mg, 411 μmol) in CH₂Cl₂ (3 mL) was added SOCl₂ (245 mg, 2.06 mmol). The mixture was stirred at 25° C. for 0.5 hr. The reaction was concentrated under reduced pressure to give 3-(3-(4-(chloromethyl)phenyl)-5-(6-methoxypyridin-3-yl)-3H-imidazo[4,5-b]pyridin-2-yl)pyrazin-2-amine (Intermediate 331, 197 mg, HCl salt) as a yellow solid. MS: m/z=444.0, 446.0 [M+H]⁺. ¹H NMR (400 MHz, Methanol-d₄) δ 9.00-8.90 (m, 2H), 8.44 (d, J=8.4 Hz, 1H), 8.14 (d, J=8.4 Hz, 1H), 7.91 (d, J=3.2 Hz, 1H), 7.72 (d, J=3.6 Hz, 1H), 7.65-7.61 (m, 2H), 7.56-7.46 (m, 3H), 4.80 (s, 2H), 4.23 (s, 3H).

### Intermediate 332: 3-(3-(4-(Chloromethyl)phenyl)-5-(6-(methoxy-d₃)pyridin-3-yl)-3H-imidazo[4,5-b]pyridin-2-yl)pyrazin-2-amine

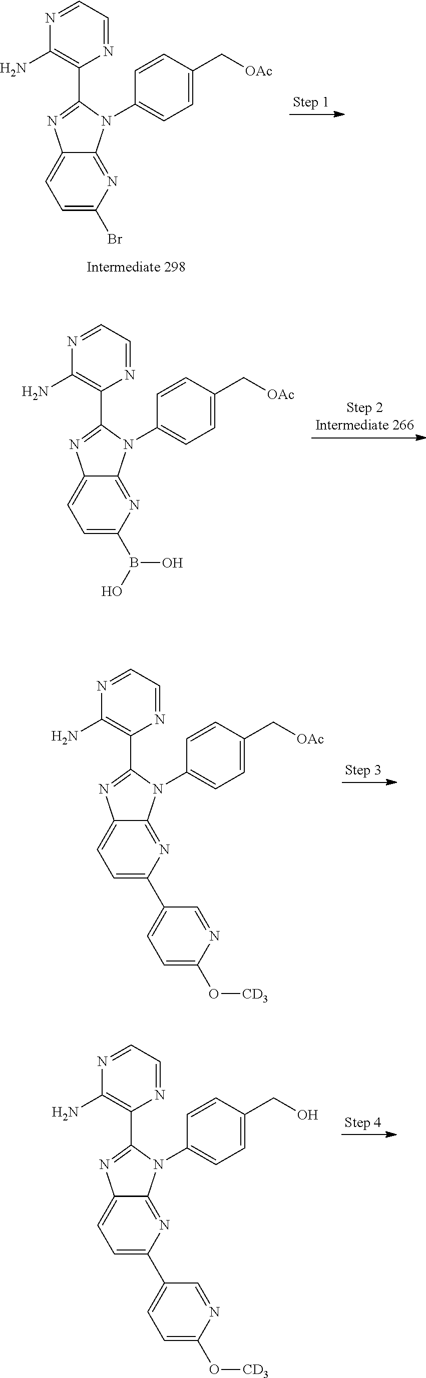 [SMILES: CC(=O)OCc1ccc(-n2c(-c3nccnc3N)nc3ccc(Br)nc32)cc1 ; CC(=O)OCc1ccc(-n2c(-c3nccnc3N)nc3ccc(B(O)O)nc32)cc1 ; [2H]C([2H])([2H])Oc1ccc(-c2ccc3nc(-c4nccnc4N)n(-c4ccc(COC(C)=O)cc4)c3n2)cn1 ; [2H]C([2H])([2H])Oc1ccc(-c2ccc3nc(-c4nccnc4N)n(-c4ccc(CO)cc4)c3n2)cn1]

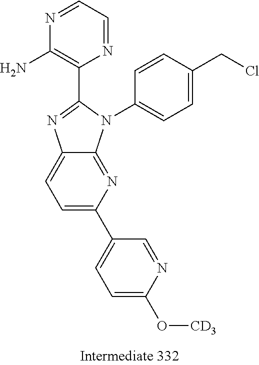 [SMILES: [2H]C([2H])([2H])Oc1ccc(-c2ccc3nc(-c4nccnc4N)n(-c4ccc(CCl)cc4)c3n2)cn1]

### Step 1: (3-(4-(Acetoxymethyl)phenyl)-2-(3-aminopyrazin-2-yl)-3H-imidazo[4,5-b]pyridin-5-yl)boronic acid

A mixture of Intermediate 298 (500 mg, 1.14 mmol), 4,4,4′,4′,5,5,5′,5′-octamethyl-2,2′-bi(1,3,2-dioxaborolane) (578 mg, 2.28 mmol), Pd(dppf)Cl₂ (83.3 mg, 114 μmol) and KOAc (335 mg, 3.41 mmol) in 1,4-dioxane (5 mL) was degassed. purged with N₂ three times, and stirred at 90° C. for 2 hr under N₂ atmosphere. (3-(4-(Acetoxymethyl)phenyl)-2-(3-aminopyrazin-2-yl)-3H-imidazo[4,5-b]pyridin-5-yl)boronic acid was obtained as a black liquid, which was used in the next step without work-up and purification. MS: m/z=405.0 [M+H]⁺.

### Step 2: 4-(2-(3-Aminopyrazin-2-yl)-5-(6-(methoxy-d₃)pyridin-3-yl)-3H-imidazo[4,5-b]pyridin-3-yl)benzyl acetate

A mixture of (3-(4-(acetoxymethyl)phenyl)-2-(3-aminopyrazin-2-yl)-3H-imidazo[4,5-b]pyridin-5-yl)boronic acid (460 mg, 1.14 mmol), Intermediate 266 (326 mg, 1.71 mmol), Cs₂CO₃ (1.11 g, 3.41 mmol) and Pd(dppf)Cl₂ (83.3 mg, 114 μmol) in 1,4-dioxane (6 mL) and H₂O (1.2 mL) was degassed, purged with N₂ three times, and stirred at 90° C. for 1 hr under N₂ atmosphere. The reaction mixture was quenched with H₂O (20 mL) and extracted with CH₂Cl₂ (15 mL×2). The combined organic layers were washed with brine (30 mL), dried over Na₂SO₄, filtered and concentrated under reduced pressure. After purified by silica gel flash chromatography (Eluent of 10%˜42% EtOAc in petroleum ether), 4-(2-(3-aminopyrazin-2-yl)-5-(6-(methoxy-d₃)pyridin-3-yl)-3H-imidazo[4,5-b]pyridin-3-yl)benzyl acetate (380 mg, yield: 71% for two steps) was obtained as a yellow solid. MS: m/z=471.1 [M+H]⁺. D %: 3D %=99.2%. ¹H NMR (400 MHz, Dimethylsulfoxide-d₆) δ 8.84 (d, J=2.4 Hz, 1H), 8.37-8.27 (m, 2H), 8.06-7.99 (m, 2H), 7.82 (br s, 2H), 7.54-7.44 (m, 5H), 6.92 (d, J=8.8 Hz, 1H), 5.19 (s, 2H), 2.13 (s, 3H).

### Step 3: (4-(2-(3-Aminopyrazin-2-yl)-5-(6-(methoxy-d₃)pyridin-3-yl)-3H-imidazo[4,5-b]pyridin-3-yl)phenyl)methanol

To a solution of 4-(2-(3-aminopyrazin-2-yl)-5-(6-(methoxy-d₃)pyridin-3-yl)-3H-imidazo[4,5-b]pyridin-3-yl)benzyl acetate (380 mg, 808 μmol) in THF (10 mL), MeOH (10 mL) and H₂O (10 mL) was added K₂CO₃ (335 mg, 4.42 mmol). The mixture was stirred at 25° C. for 1 hr. The reaction mixture was quenched with H₂O (20 mL) at 25° C. and extracted with CH₂Cl₂ (25 mL×2). The combined organic layers were washed with brine (50 mL), dried over Na₂SO₄, filtered and concentrated under reduced pressure. (4-(2-(3-Aminopyrazin-2-yl)-5-(6-(methoxy-d₃)pyridin-3-yl)-3H-imidazo[4,5-b]pyridin-3-yl)phenyl)methanol (346 mg) was obtained as a yellow solid. MS: m/z=429.0 [M+H]⁺. ¹H NMR (400 MHz, Dimethylsulfoxide-d₆) δ 8.84 (d, J=2.4 Hz, 1H), 8.38-8.26 (m, 2H), 8.02 (dd, J=5.6, 3.2 Hz, 2H), 7.79 (br s, 2H), 7.54 (d, J=2.4 Hz, 1H), 7.47-7.36 (m, 4H), 6.91 (d, J=8.8 Hz, 1H), 5.33 (br t, J=5.6 Hz, 1H), 4.62 (d, J=5.6 Hz, 2H).

### Step 4: 3-(3-(4-(Chloromethyl)phenyl)-5-(6-(methoxy-d₃)pyridin-3-yl)-3H-imidazo[4,5-b]pyridin-2-yl)pyrazin-2-amine

To a solution of (4-(2-(3-aminopyrazin-2-yl)-5-(6-(methoxy-d₃)pyridin-3-yl)-3H-imidazo[4,5-b]pyridin-3-yl)phenyl)methanol (345 mg, 805 mol) in CH₂Cl₂ (5 mL) was added SOCl₂ (479 mg, 4.03 mmol). The mixture was stirred at 25° C. for 1 hr. The reaction was concentrated under reduced pressure to give 3-(3-(4-(chloromethyl)phenyl)-5-(6-(methoxy-d₃)pyridin-3-yl)-3H-imidazo[4,5-b]pyridin-2-yl)pyrazin-2-amine (Intermediate 332, 389 mg, HCl salt) was obtained as a yellow solid. MS: m/z=447.2, 449.2 [M+H]⁺. ¹H NMR (400 MHz, Methanol-d₄) δ 8.98-8.89 (m, 2H), 8.43 (d, J=8.4 Hz, 1H), 8.13 (d, J=8.4 Hz, 1H), 7.91 (d, J=3.2 Hz, 1H), 7.72 (d, J=2.8 Hz, 1H), 7.63 (d, J=8.0 Hz, 2H), 7.54-7.44 (m, 3H), 4.80 (s, 2H).

### Intermediate 333: 2-Bromo-5-(methoxy-d₃)pyridine

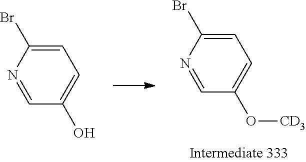 [SMILES: Oc1ccc(Br)nc1 ; [2H]C([2H])([2H])Oc1ccc(Br)nc1]

To a solution of 6-bromopyridin-3-ol (1.0 g, 5.75 mmol) in 1,4-dioxane (3 mL) were added CD₃OD (1.24 g, 34.5 mmol), PPh₃ (1.66 g, 6.32 mmol) and DIAD (1.39 g, 6.90 mmol) at 0° C. The mixture was stirred at 25° C. for 3 hr. The reaction mixture was diluted with H₂O (30 mL) and extracted with CH₂Cl₂ (25 mL×2). The combined organic layers were washed with brine (50 mL), dried over anhydrous Na₂SO₄, filtered and concentrated under reduced pressure. After purified by silica gel flash chromatography (Eluent of 0%˜15% EtOAc in petroleum ether), 2-bromo-5-(methoxy-d₃)pyridine (Intermediate 333, 1.0 g, yield: 71%) was obtained as a yellow oil. MS: m/z=190.9, 192.9 [M+H]⁺. D %: 3D %=99.3%. ¹H NMR (400 MHz, Dimethylsulfoxide-d₆) δ 8.12 (d, J=3.2 Hz, 1H), 7.55 (d, J=8.4 Hz, 1H), 7.39 (dd, J=8.8, 3.2 Hz, 1H).

### Intermediate 334: 3-(3-(4-(Chloromethyl)phenyl)-5-(5-(methoxy-d₃)pyridin-2-yl)-3H-imidazo[4,5-b]pyridin-2-yl)pyrazin-2-amine

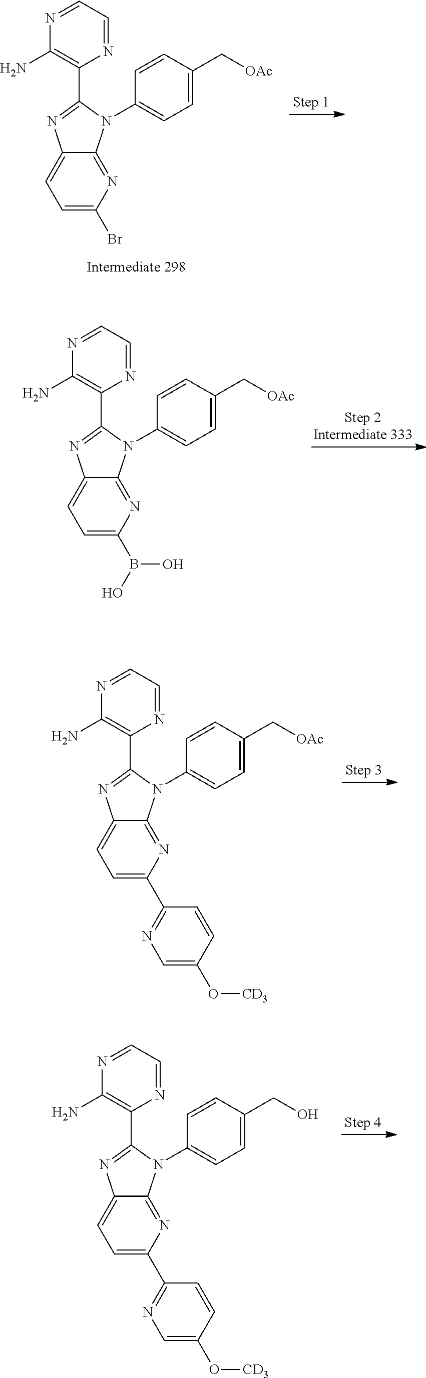 [SMILES: CC(=O)OCc1ccc(-n2c(-c3nccnc3N)nc3ccc(Br)nc32)cc1 ; CC(=O)OCc1ccc(-n2c(-c3nccnc3N)nc3ccc(B(O)O)nc32)cc1 ; [2H]C([2H])([2H])Oc1ccc(-c2ccc3nc(-c4nccnc4N)n(-c4ccc(COC(C)=O)cc4)c3n2)nc1 ; [2H]C([2H])([2H])Oc1ccc(-c2ccc3nc(-c4nccnc4N)n(-c4ccc(CO)cc4)c3n2)nc1]

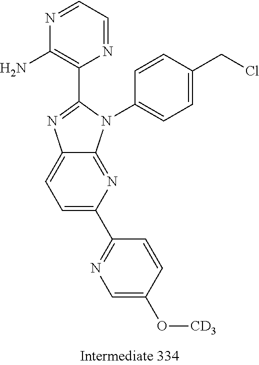 [SMILES: [2H]C([2H])([2H])Oc1ccc(-c2ccc3nc(-c4nccnc4N)n(-c4ccc(CCl)cc4)c3n2)nc1]

### Step 1: (3-(4-(Acetoxymethyl)phenyl)-2-(3-aminopyrazin-2-yl)-3H-imidazo[4,5-b]pyridin-5-yl)boronic acid

A mixture of Intermediate 298 (500 mg, 1.14 mmol), 4,4,4′,4′,5,5,5′,5′-octamethyl-2,2′-bi(1,3,2-dioxaborolane) (578 mg, 2.28 mmol), Pd(dppf)Cl₂ (83.3 mg, 114 μmol) and KOAc (335 mg, 3.41 mmol) in 1,4-dioxane (5 mL) was degassed, purged with N₂ three times, and stirred at 90° C. for 2 hr under N₂ atmosphere. (3-(4-(Acetoxymethyl)phenyl)-2-(3-aminopyrazin-2-yl)-3H-imidazo[4,5-b]pyridin-5-yl)boronic acid was obtained as a black liquid, which was used in the next step without work-up and purification. MS: m/z=405.0 [M+H]⁺.

### Step 2: 4-(2-(3-Aminopyrazin-2-yl)-5-(5-(methoxy-d₃)pyridin-2-yl)-3H-imidazo[4,5-b]pyridin-3-yl)benzyl acetate

A mixture of (3-(4-(acetoxymethyl)phenyl)-2-(3-aminopyrazin-2-yl)-3H-imidazo[4,5-b]pyridin-5-yl)boronic acid (460 mg, 1.14 mmol), Intermediate 333 (434 mg, 2.28 mmol), Cs₂CO₃ (1.11 g, 3.41 mmol) and Pd(dppf)Cl₂ (83.3 mg, 114 μmol) in 1,4-dioxane (6 mL) and H₂O (1.5 mL) was degassed, purged with N₂ three times, and stirred at 90° C. for 1 hr under N₂ atmosphere. The reaction mixture was quenched with H₂O (20 mL) and extracted with CH₂Cl₂ (25 mL×2). The combined organic layers were washed with brine (50 mL), dried over Na₂SO₄, filtered and concentrated under reduced pressure. After purified by silica gel flash chromatography (Eluent of 15%˜50% EtOAc in petroleum ether), 4-(2-(3-aminopyrazin-2-yl)-5-(5-(methoxy-d₃)pyridin-2-yl)-3H-imidazo[4,5-b]pyridin-3-yl)benzyl acetate (350 mg, yield: 62% for two steps) was obtained as a brown solid. MS: m/z=471.2 [M+H]⁺. D %: 3D %=95.5%. ¹H NMR (400 MHz, Dimethylsulfoxide-d₆) δ 8.43-8.31 (m, 3H), 8.11 (d, J=8.4 Hz, 1H), 8.02 (d, J=2.4 Hz, 1H), 7.80 (br s, 2H), 7.54-7.46 (m, 6H), 5.21 (s, 2H), 2.14 (s, 3H).

### Step 3: (4-(2-(3-Aminopyrazin-2-yl)-5-(5-(methoxy-d₃)pyridin-2-yl)-3H-imidazo[4,5-b]pyridin-3-yl)phenyl)methanol

To a solution of 4-(2-(3-aminopyrazin-2-yl)-5-(5-(methoxy-d₃)pyridin-2-yl)-3H-imidazo[4,5-b]pyridin-3-yl)benzyl acetate (350 mg, 744 μmol) in THF (10 mL), MeOH (10 mL) and H₂O (10 mL) was added K₂CO₃ (308 mg, 2.23 mmol). The mixture was stirred at 25° C. for 1 hr. The reaction mixture was quenched with H₂O (20 mL) at 25° C. and extracted with CH₂Cl₂ (25 mL×2). The combined organic layers were washed with brine (50 mL), dried over Na₂SO₄, filtered and concentrated under reduced pressure. (4-(2-(3-Aminopyrazin-2-yl)-5-(5-(methoxy-d₃)pyridin-2-yl)-3H-imidazo[4,5-b]pyridin-3-yl)phenyl)methanol (320 mg) was obtained as a yellow solid. MS: m/z=429.2 [M+H]⁺. ¹H NMR (400 MHz, Dimethylsulfoxide-d₆) δ 8.43-8.28 (m, 3H), 8.10 (d, J=8.8 Hz, 1H), 8.02 (d, J=2.4 Hz, 1H), 7.78 (br s, 2H), 7.54 (d, J=2.4 Hz, 1H), 7.50 (dd, J=8.8, 2.8 Hz, 1H), 7.47-7.43 (m, 2H), 7.41-7.37 (m, 2H), 5.36 (br s, 1H), 4.62 (s, 2H).

### Step 4: 3-(3-(4-(Chloromethyl)phenyl)-5-(5-(methoxy-d)pyridin-2-yl)-3H-imidazo[4,5-b]pyridin-2-yl)pyrazin-2-amine

To a solution of (4-(2-(3-aminopyrazin-2-yl)-5-(5-(methoxy-d₃)pyridin-2-yl)-3H-imidazo[4,5-b]pyridin-3-yl)phenyl)methanol (320 mg, 747 gmol) in CH₂Cl₂ (10 mL) was added SOCl₂ (444 mg, 3.74 mmol). The mixture was stirred at 25° C. for 1 hr. The reaction was concentrated under reduced pressure to give 3-(3-(4-(chloromethyl)phenyl)-5-(5-(methoxy-d₃)pyridin-2-yl)-3H-imidazo[4,5-b]pyridin-2-yl)pyrazin-2-amine (Intermediate 334, 360 mg, HCl salt) as a yellow solid. MS: m/z=447.1, 449.1 [M+H]⁺.

### Intermediate 335: 3-(3-(4-(Chloromethyl)phenyl)-5-(6-(fluoromethoxy)pyridin-3-yl)-3H-imidazo[4,5-b]pyridin-2-yl)pyrazin-2-amine

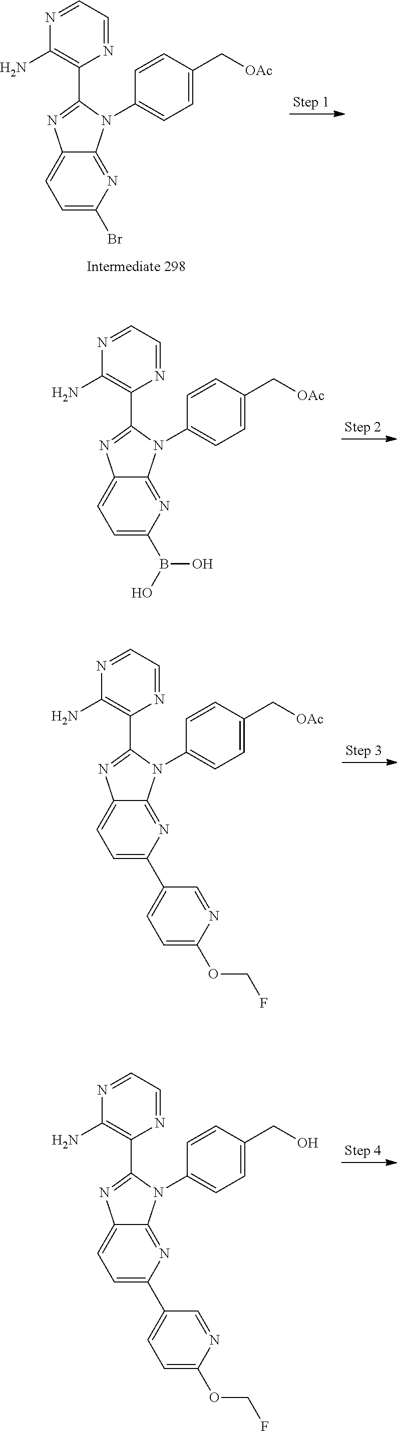 [SMILES: CC(=O)OCc1ccc(-n2c(-c3nccnc3N)nc3ccc(Br)nc32)cc1 ; CC(=O)OCc1ccc(-n2c(-c3nccnc3N)nc3ccc(B(O)O)nc32)cc1 ; CC(=O)OCc1ccc(-n2c(-c3nccnc3N)nc3ccc(-c4ccc(OCF)nc4)nc32)cc1 ; Nc1nccnc1-c1nc2ccc(-c3ccc(OCF)nc3)nc2n1-c1ccc(CO)cc1]

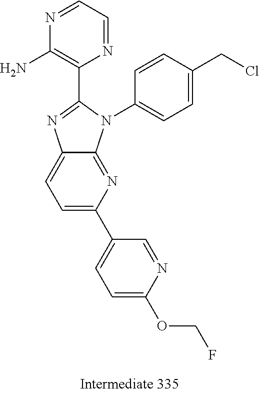 [SMILES: Nc1nccnc1-c1nc2ccc(-c3ccc(OCF)nc3)nc2n1-c1ccc(CCl)cc1]

### Step 1: (3-(4-(Acetoxymethyl)phenyl)-2-(3-aminopyrazin-2-yl)-3H-imidazo[4,5-b]pyridin-5-yl)boronic acid

A mixture of Intermediate 298 (500 mg, 1.14 mmol), 4,4,4′,4′,5,5,5′,5′-octamethyl-2,2′-bi(1,3,2-dioxaborolane) (578 mg, 2.28 mmol), KOAc (355 mg, 3.41 mmol) and Pd(dppf)Cl₂ (83.3 mg, 114 μmol) in 1,4-dioxane (5 mL) was degassed, purged with N₂ three times, and stirred at 100° C. for 3 hr under N₂ atmosphere. (3-(4-(Acetoxymethyl)phenyl)-2-(3-aminopyrazin-2-yl)-3H-imidazo[4,5-b]pyridin-5-yl)boronic acid was obtained as a black liquid, which was used in the next step without work-up and purification. MS: m/z=404.9 [M+H]⁺.

### Step 2: 4-(2-(3-Aminopyrazin-2-yl)-5-(6-(fluoromethoxy)pyridin-3-yl)-3H-imidazo[4,5-b]pyridin-3-yl)benzyl acetate

A mixture of (3-(4-(acetoxymethyl)phenyl)-2-(3-aminopyrazin-2-yl)-3H-imidazo[4,5-b]pyridin-5-yl)boronic acid (470 mg, 1.16 mmol), 5-bromo-2-(fluoromethoxy)pyridine (264 mg, 1.28 mmol), Cs₂CO₃ (1.14 g, 3.49 mmol), Pd(dppf)Cl₂ (85.1 mg, 116 μmol) in 1,4-dioxane (5 mL) and H₂O (1 mL) was degassed and purged with N₂ three times. The mixture was stirred at 90° C. for 3 hr under N₂ atmosphere. The reaction mixture was poured into H₂O (15 mL), extracted with EtOAc (15 mL×3). The combined organic layers were washed with brine (30 mL×3), dried over anhydrous Na₂SO₄, filtered and concentrated under reduced pressure. After purified by silica gel flash chromatography (Eluent of 0˜30% EtOAc in petroleum ether), 4-(2-(3-aminopyrazin-2-yl)-5-(6-(fluoromethoxy)pyridin-3-yl)-3H-imidazo[4,5-b]pyridin-3-yl)benzyl acetate (385 mg, yield: 68% for two steps) was obtained as a brown solid. MS: m/z=486.0 [M+H]⁺. ¹H NMR (400 MHz, Dimethysulfoxide-d₆) δ 8.90 (d, J=2.0 Hz, 1H), 8.51-8.27 (m, 2H), 8.16-7.98 (m, 2H), 7.82 (br s, 2H), 7.58-7.43 (m, 5H), 7.10 (d, J=8.4 Hz, 1H), 6.12 (d, J=52.4 Hz, 2H), 5.19 (s, 2H), 2.13 (s, 3H). ¹⁹F NMR (400 MHz, Dimethysulfoxide-d₆) 6-154.404.

### Step 3: (4-(2-(3-Aminopyrazin-2-yl)-5-(6-(fluoromethoxy)pyridin-3-yl)-3H-imidazo[4,5-b]pyridin-3-yl)phenyl)methanol

To a solution of 4-(2-(3-aminopyrazin-2-yl)-5-(6-(fluoromethoxy)pyridin-3-yl)-3H-imidazo[4,5-b]pyridin-3-yl)benzyl acetate (385 mg, 793 μmol) in MeOH (8 mL) and THF (8 mL) was added K₂CO₃ (219 mg, 1.59 mmol) in H₂O (2 mL). The mixture was stirred at 25° C. for 0.5 hr. The reaction mixture was concentrated under reduced pressure, diluted with H₂O (20 mL), and extracted with EtOAc (50 mL×3). The combined organic layers were dried over anhydrous Na₂SO₄, filtered and concentrated under reduced pressure. (4-(2-(3-Aminopyrazin-2-yl)-5-(6-(fluoromethoxy)pyridin-3-yl)-3H-imidazo[4,5-b]pyridin-3-yl)phenyl)methanol (360 mg, crude) was obtained as a yellow solid. MS: m/z=444.0 [M+H]⁺.

### Step 4: 3-(3-(4-(Chloromethyl)phenyl)-5-(6-(fluoromethoxy)pyridin-3-yl)-3H-imidazo[4,5-b]pyridin-2-yl)pyrazin-2-amine

To a solution of 4-(2-(3-aminopyrazin-2-yl)-5-(6-(fluoromethoxy)pyridin-3-yl)-3H-imidazo[4,5-b]pyridin-3-yl)phenyl)methanol (360 mg, 812 μmol) in CH₂Cl₂ (5 mL) was added SOCl₂ (2 mL). The mixture was stirred at 40° C. for 0.3 hr. The reaction was concentrated under reduced pressure to give 3-(3-(4-(chloromethyl)phenyl)-5-(6-(fluoromethoxy)pyridin-3-yl)-3H-imidazo[4,5-b]pyridin-2-yl)pyrazin-2-amine (Intermediate 335, 420 mg, HCl salt) was obtained as a yellow solid. MS: m/z=461.9, 463.7 [M+H]⁺.

### Intermediate 336: 3-(3-(4-(Chloromethyl)phenyl)-5-(6-(fluoromethoxy-d₂)pyridin-3-yl)-3H-imidazo[4,5-b]pyridin-2-yl)pyrazin-2-amine

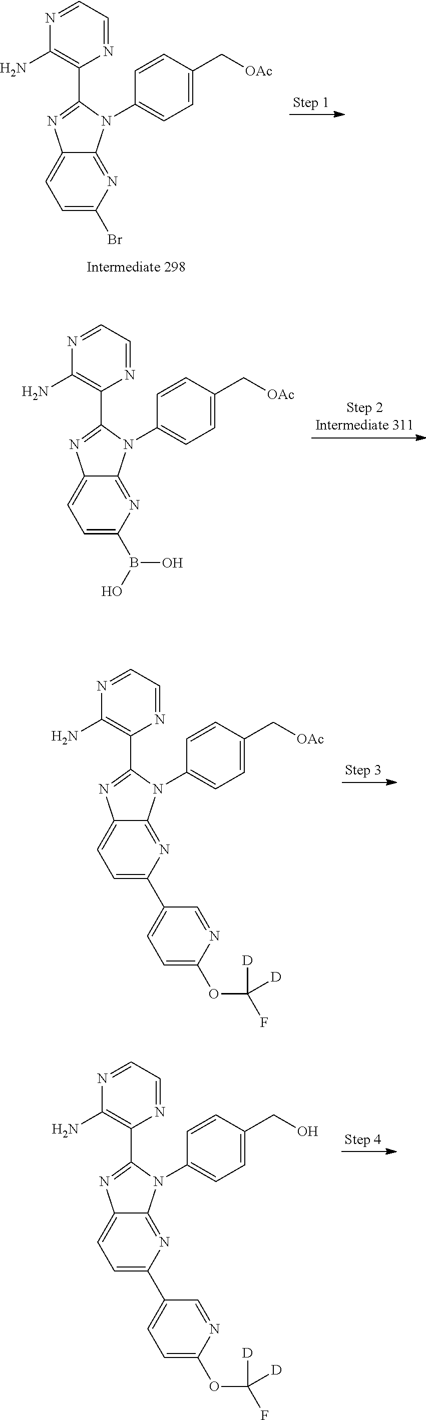 [SMILES: CC(=O)OCc1ccc(-n2c(-c3nccnc3N)nc3ccc(Br)nc32)cc1 ; CC(=O)OCc1ccc(-n2c(-c3nccnc3N)nc3ccc(B(O)O)nc32)cc1 ; [2H]C([2H])(F)Oc1ccc(-c2ccc3nc(-c4nccnc4N)n(-c4ccc(COC(C)=O)cc4)c3n2)cn1 ; [2H]C([2H])(F)Oc1ccc(-c2ccc3nc(-c4nccnc4N)n(-c4ccc(CO)cc4)c3n2)cn1]

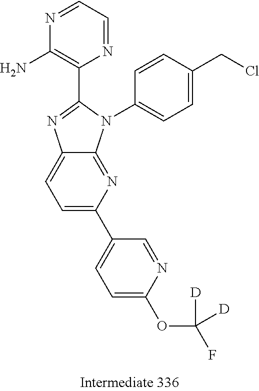 [SMILES: [2H]C([2H])(F)Oc1ccc(-c2ccc3nc(-c4nccnc4N)n(-c4ccc(CCl)cc4)c3n2)cn1]

### Step 1: (3-(4-(Acetoxymethyl)phenyl)-2-(3-aminopyrazin-2-yl)-3H-imidazo[4,5-b]pyridin-5-yl)boronic acid

A mixture of Intermediate 298 (550 mg, 1.25 mmol), 4,4,4′,4′,5,5,5′,5′-octamethyl-2,2′-bi(1,3,2-dioxaborolane) (636 mg, 2.50 mmol), Pd(dppf)Cl₂ (91.6 mg, 125 μmol) and KOAc (369 mg, 3.76 mmol) in 1,4-dioxane (12 mL) was degassed, purged with N₂ three times, and stirred at 80° C. for 16 hr under N₂ atmosphere. (3-(4-(Acetoxymethyl)phenyl)-2-(3-aminopyrazin-2-yl)-3H-imidazo[4,5-b]pyridin-5-yl)boronic acid was obtained as a black liquid, which was used in the next step without work-up and purification. MS: m/z=405.0 [M+H]⁺.

### Step 2: 4-(2-(3-Aminopyrazin-2-yl)-5-(6-(fluoromethoxy-d₂)pyridin-3-yl)-3H-imidazo[4,5-b]pyridin-3-yl)benzyl acetate

A mixture of (3-(4-(acetoxymethyl)phenyl)-2-(3-aminopyrazin-2-yl)-3H-imidazo[4,5-b]pyridin-5-yl)boronic acid (350 mg, 865 μmol), Intermediate 311 (198 mg, 952 μmol), Cs₂CO₃ (846 mg, 2.60 mmol) and Pd(dppf)Cl₂ (63.3 mg, 86.5 μmol) in 1,4-dioxane (10 mL) and H₂O (2 mL) was degassed, purged with N₂ three times, and stirred at 90° C. for 1 hr under N₂ atmosphere. The reaction mixture was quenched with H₂O (20 mL) and extracted with CH₂Cl₂ (15 mL×2). The combined organic layers were washed with brine (30 mL), dried over Na₂SO₄, filtered and concentrated under reduced pressure. After purified by silica gel flash chromatography (Eluent of 15%˜50% EtOAc in petroleum ether), 4-(2-(3-aminopyrazin-2-yl)-5-(6-(fluoromethoxy-d₂)pyridin-3-yl)-3H-imidazo[4,5-b]pyridin-3-yl)benzyl acetate (255 mg, yield: 55% for two steps) was obtained as a yellow solid. MS: m/z=488.1 [M+H]⁺. D %: 2D %=93.7%. ¹H NMR (400 MHz, Dimethylsulfoxide-d₆) δ 8.90 (d, J=2.4 Hz, 1H), 8.45-8.32 (m, 2H), 8.10-8.00 (m, 2H), 7.82 (br s, 2H), 7.54 (d, J=2.4 Hz, 1H), 7.52-7.45 (m, 4H), 7.10 (d, J=8.4 Hz, 1H), 5.19 (s, 2H), 2.13 (s, 3H). ¹⁹F NMR (400 MHz, Dimethylsulfoxide-d₆) δ −155.60.

### Step 3: (4-(2-(3-Aminopyrazin-2-yl)-5-(6-(fluoromethoxy-d₂)pyridin-3-yl)-3H-imidazo[4,5-b]pyridin-3-yl)phenyl)methanol

To a solution of 4-(2-(3-aminopyrazin-2-yl)-5-(6-(fluoromethoxy-d₂)pyridin-3-yl)-3H-imidazo[4,5-b]pyridin-3-yl)benzyl acetate (255 mg, 523 μmol) in THF (10 mL), MeOH (10 mL) and H₂O (6 mL) was added K₂CO₃ (217 mg, 1.57 mmol). The mixture was stirred at 25° C. for 1 hr. The reaction mixture was quenched with H₂O (20 mL) at 25° C. and extracted with CH₂Cl₂ (30 mL×2). The combined organic layers were washed with brine (50 mL), dried over Na₂SO₄, filtered and concentrated under reduced pressure. After purified by triturated with CH₂Cl₂ (10 mL) at 25° C. for 10 min, (4-(2-(3-aminopyrazin-2-yl)-5-(6-(fluoromethoxy-d₂)pyridin-3-yl)-3H-imidazo[4,5-b]pyridin-3-yl)phenyl)methanol (185 mg, yield: 79%) was obtained as a yellow solid. MS: m/z=446.1 [M+H]⁺. ¹H NMR (400 MHz, Dimethylsulfoxide-d₆) δ 8.90 (d, J=2.0 Hz, 1H), 8.41 (dd, J=8.8, 2.4 Hz, 1H), 8.36 (d, J=8.4 Hz, 1H), 8.07 (d, J=8.8 Hz, 1H), 8.02 (d, J=2.4 Hz, 1H), 7.79 (br s, 2H), 7.55 (d, J=2.4 Hz, 1H), 7.47-7.43 (m, 2H), 7.41-7.37 (m, 2H), 7.09 (d, J=8.8 Hz, 1H), 5.42-5.28 (m, 1H), 4.61 (br s, 2H).

### Step 4: 3-(3-(4-(Chloromethyl)phenyl)-5-(6-(fluoromethoxy-d₂)pyridin-3-yl)-3H-imidazo[4,5-b]pyridin-2-yl)pyrazin-2-amine

To a solution of (4-(2-(3-aminopyrazin-2-yl)-5-(6-(fluoromethoxy-d₂)pyridin-3-yl)-3H-imidazo[4,5-b]pyridin-3-yl)phenyl)methanol (185 mg, 415 μmol) in CH₂Cl₂ (10 mL) was added SOCl₂ (819 mg, 6.89 mmol). The mixture was stirred at 25° C. for 1 hr. The reaction was concentrated under reduced pressure to give 3-(3-(4-(chloromethyl)phenyl)-5-(6-(fluoromethoxy-d₂)pyridin-3-yl)-3H-imidazo[4,5-b]pyridin-2-yl)pyrazin-2-amine (Intermediate 336, 208 mg, HCl salt) was obtained as a yellow solid. MS: m/z=464.0, 466.0 [M+H]⁺. ¹H NMR (400 MHz, Dimethylsulfoxide-d₆) δ 8.94-8.87 (m, 1H), 8.44-8.35 (m, 2H), 8.11-8.01 (m, 2H), 7.61-7.45 (m, 5H), 7.10 (d, J=8.8 Hz, 1H), 4.89 (s, 2H).

### Intermediate 337: 3-(3-(4-(Chloromethyl)phenyl)-5-(6-cyclopropoxypyridin-3-yl)-3H-imidazo[4,5-b]pyridin-2-yl)pyridin-2-amine

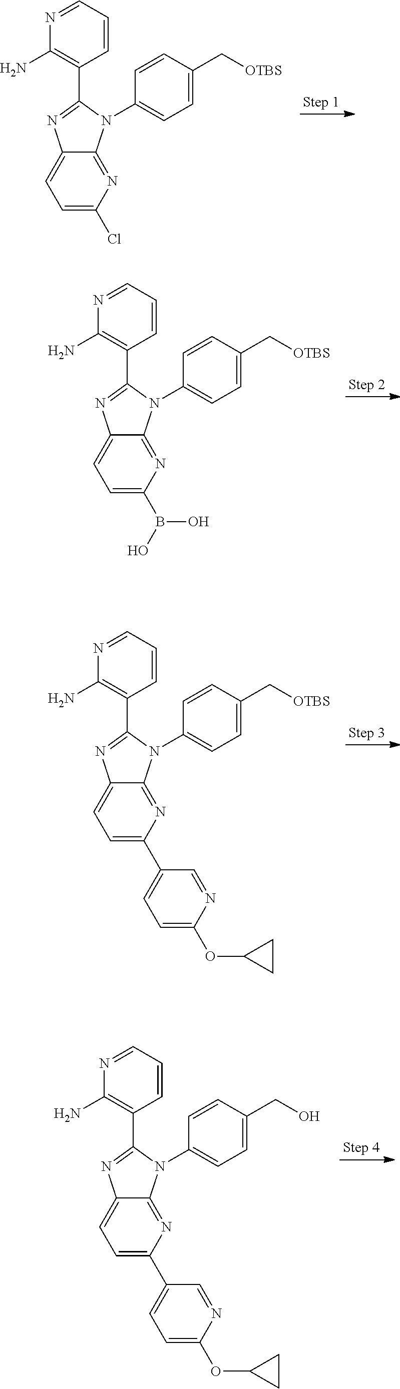 [SMILES: CC(C)(C)[Si](C)(C)OCc1ccc(-n2c(-c3cccnc3N)nc3ccc(Cl)nc32)cc1 ; CC(C)(C)[Si](C)(C)OCc1ccc(-n2c(-c3cccnc3N)nc3ccc(B(O)O)nc32)cc1 ; CC(C)(C)[Si](C)(C)OCc1ccc(-n2c(-c3cccnc3N)nc3ccc(-c4ccc(OC5CC5)nc4)nc32)cc1 ; Nc1ncccc1-c1nc2ccc(-c3ccc(OC4CC4)nc3)nc2n1-c1ccc(CO)cc1]

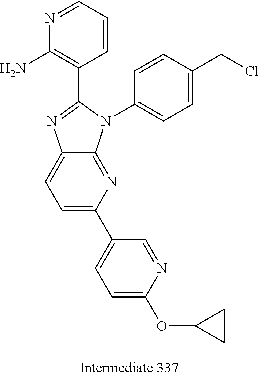 [SMILES: Nc1ncccc1-c1nc2ccc(-c3ccc(OC4CC4)nc3)nc2n1-c1ccc(CCl)cc1]

### Step 1: (2-(2-Aminopyridin-3-yl)-3-(4-(((tert-butyldimethylsilyl)oxy)methyl)phenyl)-3H-imidazo[4,5-b]pyridin-5-yl)boronic acid

To a mixture of 3-(3-(4-(((tert-butyldimethylsilyl)oxy)methyl)phenyl)-5-chloro-3H-imidazo[4,5-b]pyridin-2-yl)pyridin-2-amine (refer to Intermediate 117 for detail procedures, 500 mg, 1.07 mmol) and 4,4,4′,4′,5,5,5′,5′-octamethyl-2,2′-bi(1,3,2-dioxaborolane) (545 mg, 2.15 mmol) in 1,4-dioxane (10 mL) were added KOAc (316 mg, 3.22 mmol) and Pd(dppf)Cl₂ (78.5 mg, 107 μmol). The mixture was stirred at 100° C. for 1 hr. (2-(2-Aminopyridin-3-yl)-3-(4-(((tert-butyldimethylsilyl)oxy)methyl)phenyl)-3H-imidazo[4,5-b]pyridin-5-yl)boronic acid was obtained as a black liquid, which was used in the next step directly. MS: m/z=476.2 [M+H]⁺.

### Step 2: 3-(3-(4-(((tert-Butyldimethylsilyl)oxy)methyl)phenyl)-5-(6-cyclopropoxypyridin-3-yl)-3H-imidazo[4,5-b]pyridin-2-yl)pyridin-2-amine

To a mixture of (2-(2-aminopyridin-3-yl)-3-(4-(((tert-butyldimethylsilyl)oxy)methyl)phenyl)-3H-imidazo[4,5-b]pyridin-5-yl)boronic acid (510 mg, 1.07 mmol) and 5-bromo-2-cyclopropoxypyridine (230 mg, 1.07 mmol) in 1,4-dioxane (10 mL) and H₂O (2 mL) were added Cs₂CO₃ (1.05 g, 3.22 mmol) and Pd(dppf)Cl₂ (78.5 mg, 107 μmol), The mixture was stirred at 100° C. for 2 hr. The mixture was dilute with H₂O (50 mL) and extracted with EtOAc (50 mL×2). The combined organic layers were washed with brine (50 mL), dried over anhydrous Na₂SO₄, filtered and concentrated. After purified by silica gel flash chromatography (EtOAc in petroleum ether=0-50%), 3-(3-(4-(((tert-butyldimethylsilyl)oxy)methyl)phenyl)-5-(6-cyclopropoxypyridin-3-yl)-3H-imidazo[4,5-b]pyridin-2-yl)pyridin-2-amine (250 mg, yield: 41% for 2 steps) was obtained as a yellow solid. MS: m/z=565.3 [M+H]⁺. ¹H NMR (400 MHz, Dimethylsulfoxide-d₆) δ 8.86 (d, J=2.0 Hz, 1H), 8.35-8.24 (m, 2H), 8.03-7.97 (m, 2H), 7.50-7.44 (m, 4H), 7.20 (dd, J=7.6, 2.0 Hz, 1H), 7.03 (br s, 2H), 6.94 (d, J=8.8 Hz, 1H), 6.39 (dd, J=8.0, 4.8 Hz, 1H), 4.82 (s, 2H), 4.28-4.21 (m, 1H), 0.92 (s, 9H), 0.81-0.75 (m, 2H), 0.71-0.66 (m, 2H), 0.11 (s, 6H).

### Step 3: (4-(2-(2-Aminopyridin-3-yl)-5-(6-cyclopropoxypyridin-3-yl)-3H-imidazo[4,5-b]pyridin-3-yl)phenyl)methanol

To a mixture of 3-(3-(4-(((tert-butyldimethylsilyl)oxy)methyl)phenyl)-5-(6-cyclopropoxypyridin-3-yl)-3H-imidazo[4,5-b]pyridin-2-yl)pyridin-2-amine (230 mg, 407 μmol) in THF (3 mL) was added TBAF (1 M in THF, 611 μL). The mixture was stirred at 20° C. for 0.5 hr. The mixture was diluted with H₂O (50 mL) and extracted with EtOAc (50 mL×2). The combined organic layers were washed with brine (50 mL), dried over anhydrous Na₂SO₄, filtered and concentrated to give (4-(2-(2-aminopyridin-3-yl)-5-(6-cyclopropoxypyridin-3-yl)-3H-imidazo[4,5-b]pyridin-3-yl)phenyl)methanol (184 mg) was obtained as a brown solid. MS: m/z=451.2 [M+H]⁺.

### Step 4: 3-(3-(4-(Chloromethyl)phenyl)-5-(6-cyclopropoxypyridin-3-yl)-3H-imidazo[4,5-b]pyridin-2-yl)pyridin-2-amine

To a mixture of (4-(2-(2-aminopyridin-3-yl)-5-(6-cyclopropoxypyridin-3-yl)-3H-imidazo[4,5-b]pyridin-3-yl)phenyl)methanol (183 mg, 407 μmol) in CH₂Cl₂ (2 mL) was added SOCl₂ (88.7 μL, 1.22 mmol). The mixture was stirred at 20° C. for 0.5 hr. The mixture was filtered and concentrated to give 3-(3-(4-(chloromethyl)phenyl)-5-(6-cyclopropoxypyridin-3-yl)-3H-imidazo[4,5-b]pyridin-2-yl)pyridin-2-amine (Intermediate 337, 191 mg) as a yellow solid. MS: m/z=469.2, 471.1 [M+H]⁺.

### Intermediate 338: 3-(3-(4-(Chloromethyl)phenyl)-5-(6-isopropoxypyridin-3-yl)-3H-imidazo[4,5-b]pyridin-2-yl)pyridin-2-amine

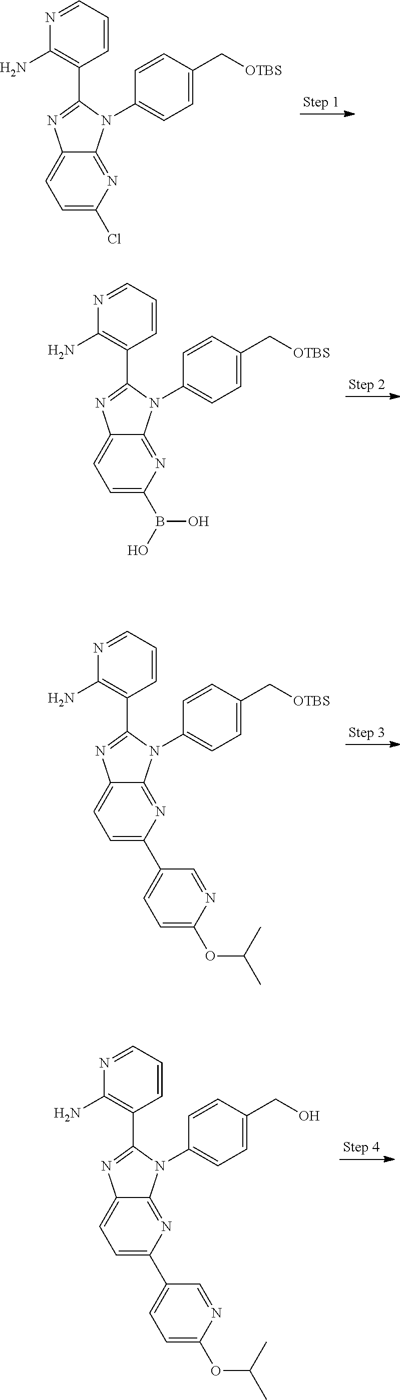 [SMILES: CC(C)(C)[Si](C)(C)OCc1ccc(-n2c(-c3cccnc3N)nc3ccc(Cl)nc32)cc1 ; CC(C)(C)[Si](C)(C)OCc1ccc(-n2c(-c3cccnc3N)nc3ccc(B(O)O)nc32)cc1 ; CC(C)Oc1ccc(-c2ccc3nc(-c4cccnc4N)n(-c4ccc(CO[Si](C)(C)C(C)(C)C)cc4)c3n2)cn1 ; CC(C)Oc1ccc(-c2ccc3nc(-c4cccnc4N)n(-c4ccc(CO)cc4)c3n2)cn1]

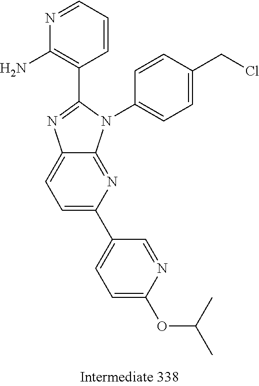 [SMILES: CC(C)Oc1ccc(-c2ccc3nc(-c4cccnc4N)n(-c4ccc(CCl)cc4)c3n2)cn1]

### Step 1: (2-(2-Aminopyridin-3-yl)-3-(4-(((tert-butyldimethylsilyl)oxy)methyl)phenyl)-3H-imidazo[4,5-b]pyridin-5-yl)boronic acid

To a mixture of 3-(3-(4-(((tert-butyldimethylsilyl)oxy)methyl)phenyl)-5-chloro-3H-imidazo[4,5-b]pyridin-2-yl)pyridin-2-amine (refer to Intermediate 117 for detail procedures, 400 mg, 784 μmol) and 4,4,4′,4′,5,5,5′,5′-octamethyl-2,2′-bi(1,3,2-dioxaborolane) (398 mg, 1.57 mmol) in 1,4-dioxane (10 mL) were added KOAc (231 mg, 2.35 mmol) and Pd(dppf)Cl₂ (57.3 mg, 78.4 μmol). The mixture was stirred at 85° C. for 1 hr. (2-(2-Aminopyridin-3-yl)-3-(4-(((tert-butyldimethylsilyl)oxy)methyl)phenyl)-3H-imidazo[4,5-b]pyridin-5-yl)boronic acid was obtained as a black liquid, which was used into the next step directly. MS: m/z=476.2 [M+H]⁺.

### Step 2: 3-(3-(4-(((Tert-butyldimethylsilyl)oxy)methyl)phenyl)-5-(6-isopropoxypyridin-3-yl)-3H-imidazo[4,5-b]pyridin-2-yl)pyridin-2-amine

To a mixture of (2-(2-aminopyridin-3-yl)-3-(4-(((tert-butyldimethylsilyl)oxy)methyl)phenyl)-3H-imidazo[4,5-b]pyridin-5-yl)boronic acid (372 mg, 782 μmol) and 5-bromo-2-isopropoxypyridine (186 mg, 861 μmol) in 1,4-dioxane (10 mL) and H₂O (2 mL) were added Cs₂CO₃ (765 mg, 2.35 mmol) and Pd(dppf)Cl₂ (57.3 mg, 78.3 μmol). The mixture was stirred at 100° C. for 1 hr. The reaction mixture was quenched with H₂O (50 mL) and extracted with EtOAc (50 mL×2). The combined organic layers were washed with brine (50 mL), dried over anhydrous Na₂SO₄, filtered and concentrated. After purified by silica gel flash chromatography (EtOAc in petroleum ether=0-50%), 3-(3-(4-(((tert-butyldimethylsilyl)oxy)methyl)phenyl)-5-(6-isopropoxypyridin-3-yl)-3H-imidazo[4,5-b]pyridin-2-yl)pyridin-2-amine (300 mg, yield: 68% for two steps) was obtained as a yellow solid. MS: m/z=567.3 [M+H]⁺.

### Step 3: (4-(2-(2-Aminopyridin-3-yl)-5-(6-isopropoxypyridin-3-yl)-3H-imidazo[4,5-b]pyridin-3-yl)phenyl)methanol

To a mixture of 3-(3-(4-(((tert-butyldimethylsilyl)oxy)methyl)phenyl)-5-(6-isopropoxypyridin-3-yl)-3H-imidazo[4,5-b]pyridin-2-yl)pyridin-2-amine (250 mg, 441 μmol) in THF (4 mL) was added TBAF (1 M in THF) (1 M, 662 μL). The mixture was stirred at 20° C. for 0.5 hr. The reaction mixture was quenched with H₂O (50 mL) and extracted with EtOAc (50 mL×2). The combined organic layers were washed with brine (20 mL), dried over anhydrous Na₂SO₄, filtered and concentrated under reduced pressure. (4-(2-(2-Aminopyridin-3-yl)-5-(6-isopropoxypyridin-3-yl)-3H-imidazo[4,5-b]pyridin-3-yl)phenyl)methanol (199.60 mg) was obtained as a brown solid which was used in the next step without purification. MS: m/z=453.2 [M+H]⁺. ¹H NMR (400 MHz, Dimethylsulfoxide-d₆) δ 8.83 (d, J=2.4 Hz, 1H), 8.30-8.22 (m, 2H), 8.03-7.95 (m, 2H), 7.51-7.43 (m, 4H), 7.26-7.16 (m, 1H), 7.00 (br s, 2H), 6.83 (d, J=8.8 Hz, 1H), 6.42 (dd, J=7.6, 4.8 Hz, 1H), 5.36 (t, J=6.0 Hz, 1H), 5.28-5.24 (m, 1H), 4.60 (d, J=6.0 Hz, 2H), 1.32-1.29 (m, 6H).

### Step 4: 3-(3-(4-(Chloromethyl)phenyl)-5-(6-isopropoxypyridin-3-yl)-3H-imidazo[4,5-b]pyridin-2-yl)pyridin-2-amine

To a mixture of (4-(2-(2-aminopyridin-3-yl)-5-(6-isopropoxypyridin-3-yl)-3H-imidazo[4,5-b]pyridin-3-yl)phenyl)methanol (199 mg, 441 μmol) in CH₂Cl₂ (2 mL) was added SOCl₂ (96.11 μL, 1.32 mmol). The mixture was stirred at 20° C. for 1 hr. The reaction was concentrated under reduced pressure. 3-(3-(4-(Chloromethyl)phenyl)-5-(6-isopropoxypyridin-3-yl)-3H-imidazo[4,5-b]pyridin-2-yl)pyridin-2-amine (Intermediate 338, 207 mg, 440 μmol) was obtained as a yellow solid. MS: m/z=471.2, 473.1 [M+H]⁺.

### Intermediate 339: 3-(3-(4-(Chloromethyl)phenyl)-5-(5-cyclopropoxypyridin-2-yl)-3H-imidazo[4,5-b]pyridin-2-yl)pyridin-2-amine

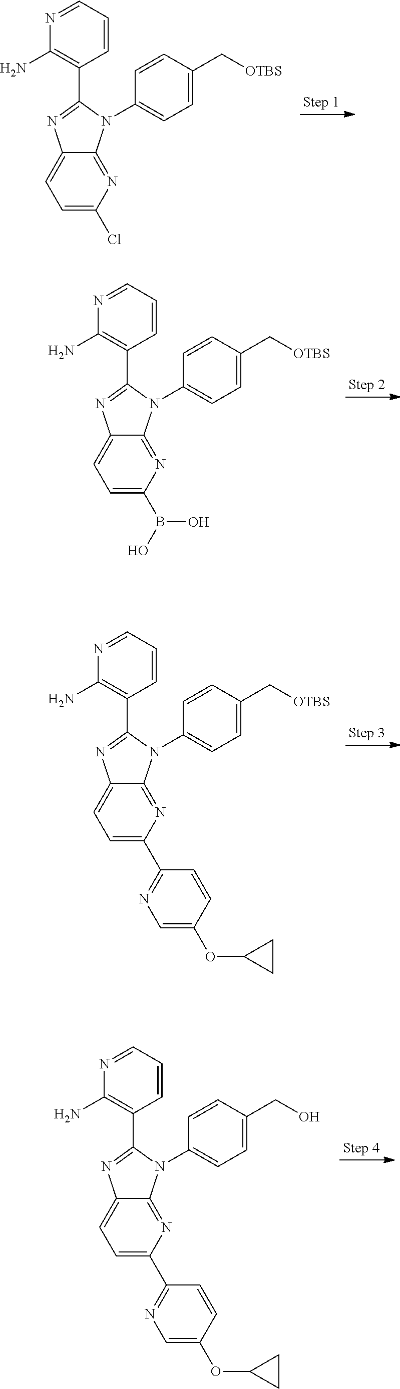 [SMILES: CC(C)(C)[Si](C)(C)OCc1ccc(-n2c(-c3cccnc3N)nc3ccc(Cl)nc32)cc1 ; CC(C)(C)[Si](C)(C)OCc1ccc(-n2c(-c3cccnc3N)nc3ccc(B(O)O)nc32)cc1 ; CC(C)(C)[Si](C)(C)OCc1ccc(-n2c(-c3cccnc3N)nc3ccc(-c4ccc(OC5CC5)cn4)nc32)cc1 ; Nc1ncccc1-c1nc2ccc(-c3ccc(OC4CC4)cn3)nc2n1-c1ccc(CO)cc1]

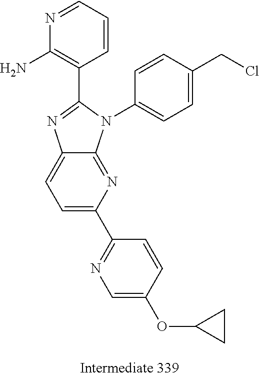 [SMILES: Nc1ncccc1-c1nc2ccc(-c3ccc(OC4CC4)cn3)nc2n1-c1ccc(CCl)cc1]

### Step 1: (2-(2-Aminopyridin-3-yl)-3-(4-(((tert-butyldimethylsilyl)oxy)methyl)phenyl)-3H-imidazo[4,5-b]pyridin-5-yl)boronic acid

To a solution of 3-(3-(4-(((tert-butyldimethylsilyl)oxy)methyl)phenyl)-5-chloro-3H-imidazo[4,5-b]pyridin-2-yl)pyridin-2-amine (refer to Intermediate 117 for detail procedures, 500 mg, 1.07 mmol) and 4,4,4′,4′,5,5,5′,5′-octamethyl-2,2′-bi(1,3,2-dioxaborolane) (409 mg, 1.61 mmol) in 1,4-dioxane (10 mL) were added Pd(dppf)Cl₂ (78.5 mg, 107 μmol) and KOAc (316 mg, 3.22 mmol). The mixture was stirred at 85° C. for 2 hr under N₂ atmosphere. (2-(2-Aminopyridin-3-yl)-3-(4-(((tert-butyldimethylsilyl)oxy)methyl)phenyl)-3H-imidazo[4,5-b]pyridin-5-yl)boronic acid was obtained as a black liquid, which was used in the next step without work-up and purification. MS: m/z=476.2 [M+H]⁺.

### Step 2: 3-(3-(4-(((Tert-butyldimethylsilyl)oxy)methyl)phenyl)-5-(5-cyclopropoxypyridin-2-yl)-3H-imidazo[4,5-b]pyridin-2-yl)pyridin-2-amine

To a solution of (2-(2-aminopyridin-3-yl)-3-(4-(((tert-butyldimethylsilyl)oxy)methyl)phenyl)-3H-imidazo[4,5-b]pyridin-5-yl)boronic acid (510 mg, 1.07 mmol) and 2-bromo-5-cyclopropoxypyridine (230 mg, 1.07 mmol) in 1,4-dioxane (6 mL) and H₂O (1 mL) were added Cs₂CO₃ (1.05 g, 3.22 mmol) and Pd(dppf)Cl₂ (78.5 mg, 107 μmol). The mixture was stirred at 100° C. for 2 hr under N₂ atmosphere. The mixture was diluted with H₂O (10 mL) and extracted with EtOAc (10 mL×2). The combined organic layers were dried over anhydrous Na₂SO₄, filtered and concentrated. After purified by silica gel flash chromatography (Eluent of 0%˜50% EtOAc in petroleum ether), 3-(3-(4-(((tert-butyldimethylsilyl)oxy)methyl)phenyl)-5-(5-cyclopropoxypyridin-2-yl)-3H-imidazo[4,5-b]pyridin-2-yl)pyridin-2-amine (320 mg, yield: 53% for two steps) was obtained as a light yellow oil. MS: m/z=565.3 [M+H]⁺. ¹H NMR (400 MHz, Dimethylsulfoxide-d₆) δ 8.45 (d, J=2.8 Hz, 1H), 8.36 (d, J=8.4 Hz, 1H), 8.7 (d, J=8.4 Hz, 1H), 8.13 (d, J=8.8 Hz, 1H), 8.00 (dd, 1=4.8, 2.0 Hz, 1H), 7.59 (dd, J=8.8, 2.8 Hz, 1H), 7.52-7.47 (m, 4H), 7.20 (dd, J=7.6, 1.6 Hz, 1H), 7.02 (br s, 2H), 6.39 (dd, J=7.6, 4.8 Hz, 1H), 4.83 (s, 2H), 4.04-3.98 (m, 1H), 0.93 (s, 9H), 0.86-0.81 (m, 2H), 0.75-0.67 (m, 2H), 0.12 (m, 6H).

### Step 3: (4-(2-(2-Aminopyridin-3-yl)-5-(5-cyclopropoxypyridin-2-yl)-3H-imidazo[4,5-b]pyridin-3-yl)phenyl)methanol

To a solution of 3-(3-(4-(((tert-butyldimethylsilyl)oxy)methyl)phenyl)-5-(5-cyclopropoxypyridin-2-yl)-3H-imidazo[4,5-b]pyridin-2-yl)pyridin-2-amine (200 mg, 354 μmol) in THF (2 mL) was added TBAF (1 M in THF, 354.14 μL). The mixture was stirred at 25° C. for 0.5 hr. The mixture was diluted with H₂O (10 mL) and extracted with EtOAc (10 mL×2). The combined organic layers were dried over anhydrous Na₂SO₄, filtered and concentrated to give (4-(2-(2-aminopyridin-3-yl)-5-(5-cyclopropoxypyridin-2-yl)-3H-imidazo[4,5-b]pyridin-3-yl)phenyl)methanol (160 mg) as a light yellow oil. MS: m/z=451.2 [M+H]⁺

### Step 4: 3-(3-(4-(Chloromethyl)phenyl)-5-(5-cyclopropoxypyridin-2-yl)-3H-imidazo[4,5-b]pyridin-2-yl)pyridin-2-amine

To a solution of (4-(2-(2-aminopyridin-3-yl)-5-(5-cyclopropoxypyridin-2-yl)-3H-imidazo[4,5-b]pyridin-3-yl)phenyl)methanol (160 mg, 354 μmol) in CH₂Cl₂ (2 mL) was added SOCl₂ (126 mg, 1.06 mmol). The mixture was stirred at 25° C. for 1 hr. The reaction was concentrated to give 3-(3-(4-(chloromethyl)phenyl)-5-(5-cyclopropoxypyridin-2-yl)-3H-imidazo[4,5-b]pyridin-2-yl)pyridin-2-amine (Intermediate 339, 166 mg, HCl salt) as a light yellow solid. MS: m/z=469.2, 471.2 [M+H]⁺.

### Intermediate 340: 3-(3-(4-(Chloromethyl)phenyl)-5-(5-isopropoxypyridin-2-yl)-3H-imidazo[4,5-b]pyridin-2-yl)pyridin-2-amine

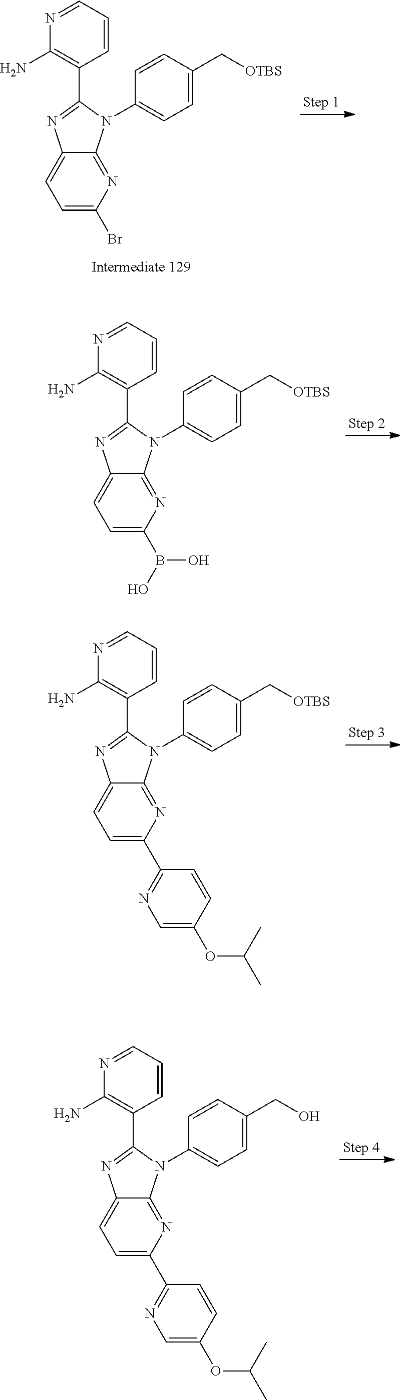

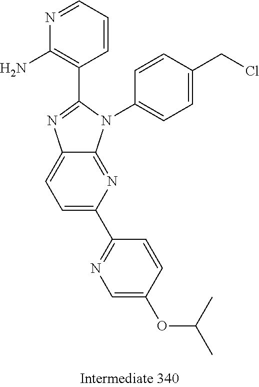 [SMILES: CC(C)Oc1ccc(-c2ccc3nc(-c4cccnc4N)n(-c4ccc(CCl)cc4)c3n2)nc1]

### Step 1: (2-(2-Aminopyridin-3-yl)-3-(4-(((tert-butyldimethylsilyl)oxy)methyl)phenyl)-3H-imidazo[4,5-b]pyridin-5-yl)boronic acid

To a solution of Intermediate 129 (400 mg, 784 μmol) and 4,4,4′,4′,5,5,5′,5′-octamethyl-2,2′-bi(1,3,2-dioxaborolane) (398 mg, 1.57 mmol) in 1,4-dioxane (4 mL) were added KOAc (231 mg, 2.35 mmol) and Pd(dppf)Cl₂ (57.3 mg, 78.4 μmol). The mixture was stirred at 85° C. for 2 hr under N₂. (2-(2-Aminopyridin-3-yl)-3-(4-(((tert-butyldimethylsilyl)oxy)methyl)phenyl)-3H-imidazo[4,5-b]pyridin-5-yl)boronic acid was obtained as a light yellow liquid, which was used in the next step without work up and purification. MS: m/z=476.3 [M+H]⁺.

### Step 2: 3-(3-(4-(((tert-Butyldimethylsilyl)oxy)methyl)phenyl)-5-(5-isopropoxypyridin-2-yl)-3H-imidazo[4,5-b]pyridin-2-yl)pyridin-2-amine

To a solution of (2-(2-aminopyridin-3-yl)-3-(4-(((tert-butyldimethylsilyl)oxy)methyl)phenyl)-3H-imidazo[4,5-b]pyridin-5-yl)boronic acid (203 mg, 940 μmol), 2-bromo-5-isopropoxypyridine (220 mg, 1.06 mmol) in 1,4-dioxane (4 mL) and H₂O (0.8 mL) were added Cs₂CO₃ (766 mg, 2.35 mmol) and Pd(dppf)Cl₂ (57.3 mg, 78.4 μmol). The mixture was stirred at 100° C. for 2 hr under N₂. The mixture was diluted with H₂O (10 mL) and extracted with EtOAc (10 mL×2). The combined organic layers were dried over anhydrous Na₂SO₄, filtered and concentrated. After purified by silica gel flash chromatography (Eluent of 0%˜40% EtOAc in petroleum ether), 3-(3-(4-(((tert-butyldimethylsilyl)oxy)methyl)phenyl)-5-(5-isopropoxypyridin-2-yl)-3H-imidazo[4,5-b]pyridin-2-yl)pyridin-2-amine (270 mg, yield: 60.8% for two steps) was obtained as a yellow oil. MS: m/z=567.3 [M+H]⁺.

### Step 3: (4-(2-(2-Aminopyridin-3-yl)-5-(5-isopropoxypyridin-2-yl)-3H-imidazo[4,5-b]pyridin-3-yl)phenyl)methanol

To a solution of 3-(3-(4-(((tert-butyldimethylsilyl)oxy)methyl)phenyl)-5-(5-isopropoxypyridin-2-yl)-3H-imidazo[4,5-b]pyridin-2-yl)pyridin-2-amine (240 mg, 424 μmol) in THF (3 mL) was added TBAF (1 M in THF, 551 μL). The mixture was stirred at 25° C. for 0.5 hr. The mixture was diluted with H₂O (10 mL) and extracted with EtOAc (5 mL×2). The combined organic layers were dried over anhydrous Na₂SO₄, filtered and concentrated. (4-(2-(2-Aminopyridin-3-yl)-5-(5-isopropoxypyridin-2-yl)-3H-imidazo[4,5-b]pyridin-3-yl)phenyl)methanol (192 mg) was obtained as light yellow oil. MS: m/z=453.2 [M+H]⁺.

### Step 4: 3-(3-(4-(Chloromethyl)phenyl)-5-(5-isopropoxypyridin-2-yl)-3H-imidazo[4,5-b]pyridin-2-yl)pyridin-2-amine

To a solution of (4-(2-(2-aminopyridin-3-yl)-5-(5-isopropoxypyridin-2-yl)-3H-imidazo[4,5-b]pyridin-3-yl)phenyl)methanol (192 mg, 424 μmol) in CH₂Cl₂ (1 mL) was added SOCl₂ (151 mg, 1.27 mmol). The mixture was stirred at 25° C. for 1 hr. The reaction was concentrated to give 3-(3-(4-(Chloromethyl)phenyl)-5-(5-isopropoxypyridin-2-yl)-3H-imidazo[4,5-b]pyridin-2-yl)pyridin-2-amine (Intermediate 340, 199 mg) as a light yellow solid. MS: m/z=471.3, 473.3 [M+H]⁺.

### Intermediate 341: 3-(3-(4-(Chloromethyl)phenyl)-5-(6-cyclopropylpyridin-3-yl)-3H-imidazo[4,5-b]pyridin-2-yl)pyridin-2-amine

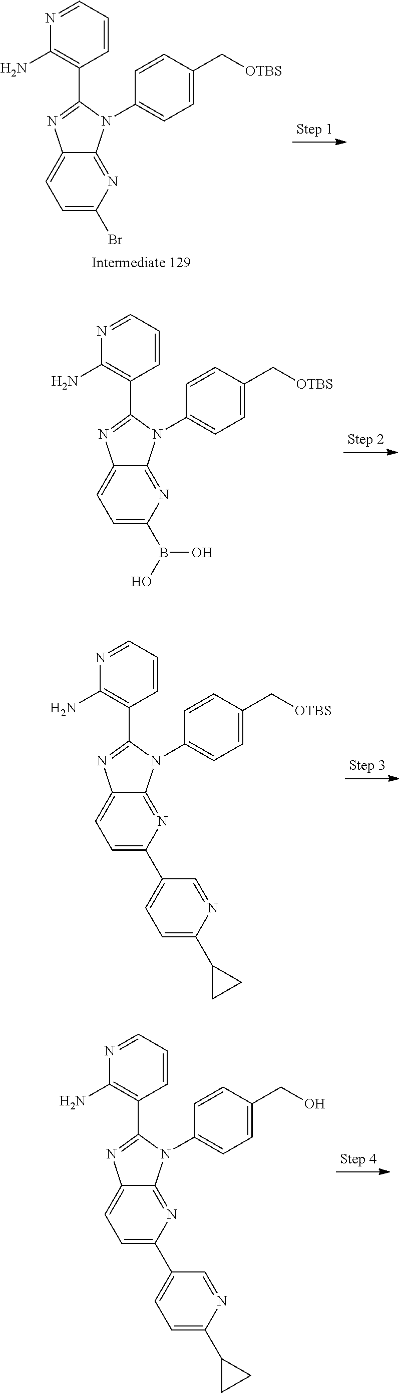 [SMILES: CC(C)(C)[Si](C)(C)OCc1ccc(-n2c(-c3cccnc3N)nc3ccc(Br)nc32)cc1 ; CC(C)(C)[Si](C)(C)OCc1ccc(-n2c(-c3cccnc3N)nc3ccc(B(O)O)nc32)cc1 ; CC(C)(C)[Si](C)(C)OCc1ccc(-n2c(-c3cccnc3N)nc3ccc(-c4ccc(C5CC5)nc4)nc32)cc1 ; Nc1ncccc1-c1nc2ccc(-c3ccc(C4CC4)nc3)nc2n1-c1ccc(CO)cc1]

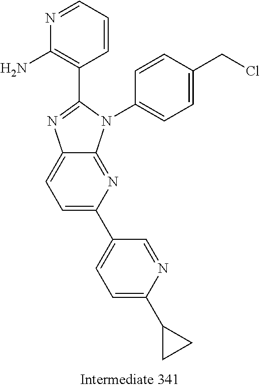 [SMILES: Nc1ncccc1-c1nc2ccc(-c3ccc(C4CC4)nc3)nc2n1-c1ccc(CCl)cc1]

### Step 1: (2-(2-Aminopyridin-3-yl)-3-(4-(((tert-butyldimethylsilyl)oxy)methyl)phenyl)-3H-imidazo[4,5-b]pyridin-5-yl)boronic acid

To a solution of Intermediate 129 (500 mg, 1.07 mmol) and 4,4,4′,4′,5,5,5′,5′-octamethyl-2,2′-bi(1,3,2-dioxaborolane) (409 mg, 1.61 mmol) in 1,4-dioxane (10 mL) were added Pd(dppf)Cl₂ (78.5 mg, 107 μmol) and KOAc (316 mg, 3.22 mmol). The mixture was stirred at 100° C. for 2 hr under N₂ atmosphere. (2-(2-Aminopyridin-3-yl)-3-(4-(((tert-butyldimethylsilyl)oxy)methyl)phenyl)-3H-imidazo[4,5-b]pyridin-5-yl)boronic acid was obtained as a black liquid, which was used in the next step without work-up and purification. MS: m/z=476.2 [M+H]⁺.

### Step 2: 3-(3-(4-(((Tert-butyldimethylsilyl)oxy)methyl)phenyl)-5-(6-cyclopropylpyridin-3-yl)-3H-imidazo[4,5-b]pyridin-2-yl)pyridin-2-amine

To a solution of (2-(2-aminopyridin-3-yl)-3-(4-(((tert-butyldimethylsilyl)oxy)methyl)phenyl)-3H-imidazo[4,5-b]pyridin-5-yl)boronic acid (510 mg, 1.07 mmol) and 5-bromo-2-cyclopropylpyridine (255 mg, 1.29 mmol) in 1,4-dioxane (10 mL) and H₂O (2 mL) were added Cs₂CO₃ (1.05 g, 3.22 mmol) and Pd(dppf)Cl₂ (78.5 mg, 107 mol). The mixture was stirred at 100° C. for 2 hr under N₂ atmosphere. The mixture was diluted with H₂O (10 mL) and extracted with EtOAc (5 mL×2). The combined organic layers were dried over anhydrous Na₂SO₄, filtered and concentrated. After purified by silica gel flash chromatography (Eluent of 0%˜50% EtOAc in petroleum ether), 3-(3-(4-(((tert-butyldimethylsilyl)oxy)methyl)phenyl)-5-(6-cyclopropylpyridin-3-yl)-3H-imidazo[4,5-b]pyridin-2-yl)pyridin-2-amine (300 mg, yield: 51% for two steps) was obtained as a yellow oil. MS: m/z=549.3 [M+H]⁺.

### Step 3: (4-(2-(2-Aminopyridin-3-yl)-5-(6-cyclopropylpyridin-3-yl)-3H-imidazo[4,5-b]pyridin-3-yl)phenyl)methanol

To a solution of 3-(3-(4-(((tert-butyldimethylsilyl)oxy)methyl)phenyl)-5-(6-cyclopropylpyridin-3-yl)-3H-imidazo[4,5-b]pyridin-2-yl)pyridin-2-amine (300 mg, 547 μmol) in THF (2 mL) was added TBAF (1 M in THF, 711 μL). The mixture was stirred at 25° C. for 0.5 hr. The mixture was diluted with H₂O (10 mL) and extracted with EtOAc (10 mL×2). The combined organic layers were dried over anhydrous Na₂SO₄, filtered and concentrated. (4-(2-(2-Aminopyridin-3-yl)-5-(6-cyclopropylpyridin-3-yl)-3H-imidazo[4,5-b]pyridin-3-yl)phenyl)methanol (238 mg) was obtained as a light yellow oil. MS: m/z=435.2 [M+H]+

### Step 4: 3-(3-(4-(Chloromethyl)phenyl)-5-(6-cyclopropylpyridin-3-yl)-3H-imidazo[4,5-b]pyridin-2-yl)pyridin-2-amine

To a solution of (4-(2-(2-aminopyridin-3-yl)-5-(6-cyclopropylpyridin-3-yl)-3H-imidazo[4,5-b]pyridin-3-yl)phenyl)methanol (238 mg, 547 μmol) in CH₂Cl₂ (2 mL) was added SOCl₂ (195 mg, 1.64 mmol). The mixture was stirred at 25° C. for 1 hr. The reaction was concentrated. 3-(3-(4-(Chloromethyl)phenyl)-5-(6-cyclopropylpyridin-3-yl)-3H-imidazo[4,5-b]pyridin-2-yl)pyridin-2-amine (Intermediate 341, 248 mg, HCl salt) was obtained as a light yellow solid. MS: m/z=453.2, 455.2 [M+H]⁺. ¹H NMR (400 MHz, Dimethylsulfoxide-d₆) δ 9.15 (s, 1H), 8.90 (d, J=8.4 Hz, 1H), 8.48 (d, J=8.4 Hz, 2H), 8.31 (d, J=8.4 Hz, 1H), 8.17 (d, J=5.6 Hz, 1H), 7.93 (d, J=7.2 Hz, 1H), 7.72 (d, J=8.8 Hz, 1H), 7.68-7.59 (m, 4H), 6.92 (t, J=6.8 Hz, 1H), 4.88 (s, 2H), 1.60-1.54 (m, 1H), 1.41-1.33 (m, 2H), 1.26-1.20 (m, 2H).

### Intermediate 342: 3-(3-(4-(Chloromethyl)phenyl)-5-(6-isopropylpyridin-3-yl)-3H-imidazo[4,5-b]pyridin-2-yl)pyridin-2-amine

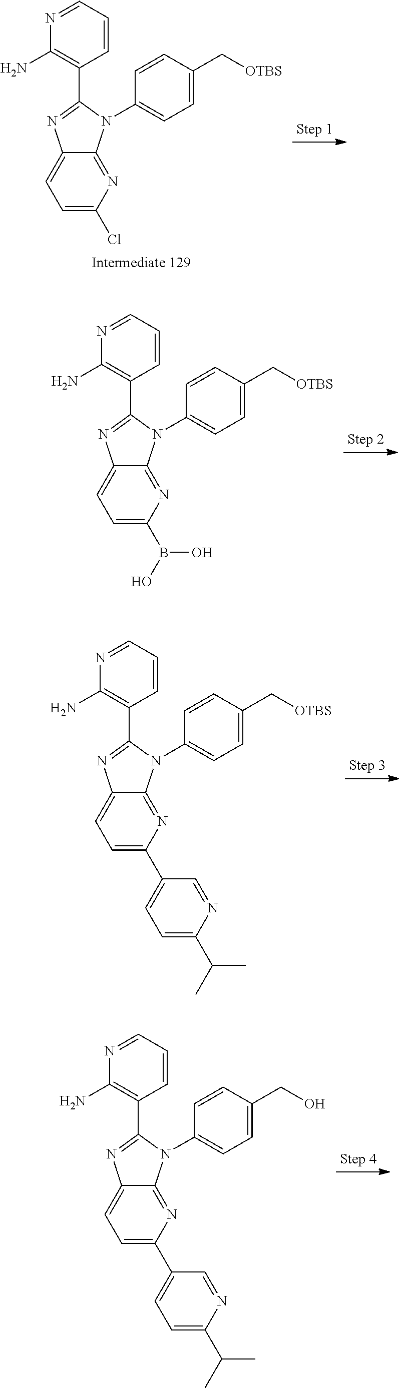 [SMILES: CC(C)(C)[Si](C)(C)OCc1ccc(-n2c(-c3cccnc3N)nc3ccc(Cl)nc32)cc1 ; CC(C)(C)[Si](C)(C)OCc1ccc(-n2c(-c3cccnc3N)nc3ccc(B(O)O)nc32)cc1 ; CC(C)c1ccc(-c2ccc3nc(-c4cccnc4N)n(-c4ccc(CO[Si](C)(C)C(C)(C)C)cc4)c3n2)cn1 ; CC(C)c1ccc(-c2ccc3nc(-c4cccnc4N)n(-c4ccc(CO)cc4)c3n2)cn1]

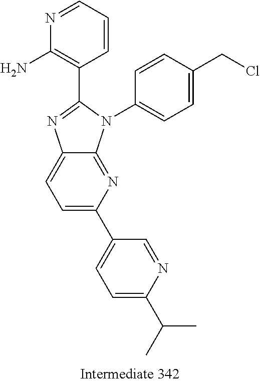 [SMILES: CC(C)c1ccc(-c2ccc3nc(-c4cccnc4N)n(-c4ccc(CCl)cc4)c3n2)cn1]

### Step 1: (2-(2-Aminopyridin-3-yl)-3-(4-(((tert-butyldimethylsilyl)oxy)methyl)phenyl)-3H-imidazo[4,5-b]pyridin-5-yl)boronic acid

To a solution of 3-(3-(4-(((tert-butyldimethylsilyl)oxy)methyl)phenyl)-5-chloro-3H-imidazo[4,5-b]pyridin-2-yl)pyridin-2-amine (500 mg, 1.07 mmol) and 4,4,4′,4′,5,5,5′,5′-octamethyl-2,2′-bi(1,3,2-dioxaborolane) (545 mg, 2.15 mmol) in 1,4-dioxane (15 mL) were added Pd(dppf)Cl₂ (78.5 mg, 107 μmol) and KOAc (316 mg, 3.22 mmol) at 20° C. The mixture was stirred at 110° C. for 2 hr under N₂. (2-(2-Aminopyridin-3-yl)-3-(4-(((tert-butyldimethylsilyl)oxy)methyl)phenyl)-3H-imidazo[4,5-b]pyridin-5-yl)boronic acid was obtained as a black liquid, which was used in the next step directly. MS: m/z=476.2 [M+H]⁺.

### Step 2: 3-(3-(4-(((tert-Butyldimethylsilyl)oxy)methyl)phenyl)-5-(6-isopropylpyridin-3-yl)-3H-imidazo[4,5-b]pyridin-2-yl)pyridin-2-amine

To a solution of (2-(2-aminopyridin-3-yl)-3-(4-(((tert-butyldimethylsilyl)oxy)methyl)phenyl)-3H-imidazo[4,5-b]pyridin-5-yl)boronic acid (510 mg, 1.07 mmol) and 5-bromo-2-isopropylpyridine (258 mg, 1.29 mmol) in 1,4-dioxane (15 mL) and H₂O (3 mL) were added Pd(dppf)Cl₂ (78.5 mg, 107 μmol) and Cs₂CO₃ (1.05 g, 3.22 mmol) at 20° C. The mixture was stirred at 100° C. for 2 hr under N₂. The mixture was diluted with H₂O (30 mL) and extracted with EtOAc (30 mL×2). The combined organic layers were washed with brine (30 mL), dried over anhydrous Na₂SO₄, filtered and concentrated. After purified by silica gel flash chromatography (30˜100% EtOAc in petroleum ether), 3-(3-(4-(((tert-butyldimethylsilyl)oxy)methyl)phenyl)-5-(6-isopropylpyridin-3-yl)-3H-imidazo[4,5-b]pyridin-2-yl)pyridin-2-amine (250 mg, 454 μmol, yield: 42% for 2 steps) was obtained as a brown solid. MS: m/z=551.3 [M+H]⁺. ¹H NMR (400 MHz, Dimethylsulfoxide-d₆) δ 9.13-9.09 (m, 1H), 8.32-8.23 (m, 2H), 8.04-7.98 (m, 2H), 7.52-7.46 (m, 4H), 7.35 (d, J=8.4 Hz, 1H), 7.20 (dd, J=7.6, 2.0 Hz, 1H), 7.03 (s, 2H), 6.42-6.36 (m, 1H), 4.82 (s, 2H), 3.10-2.99 (m, 1H), 1.24 (d, J=6.8 Hz, 6H), 0.92 (s, 9H), 0.11 (s, 6H).

### Step 3: (4-(2-(2-Aminopyridin-3-yl)-5-(6-isopropylpyridin-3-yl)-3H-imidazo[4,5-b]pyridin-3-yl)phenyl)methanol

To a solution of 3-(3-(4-(((tert-butyldimethylsilyl)oxy)methyl)phenyl)-5-(6-isopropylpyridin-3-yl)-3H-imidazo[4,5-b]pyridin-2-yl)pyridin-2-amine (250 mg, 454 μmol) in THF (6 mL) was added TBAF (1 M in THF, 681 μL). The reaction mixture was stirred at 20° C. for 0.5 hr. The mixture was diluted with H₂O (20 mL) and extracted with EtOAc (20 mL×2). The combined organic layers were washed with brine (20 mL), dried over anhydrous Na₂SO₄, filtered and concentrated. (4-(2-(2-Aminopyridin-3-yl)-5-(6-isopropylpyridin-3-yl)-3H-imidazo[4,5-b]pyridin-3-yl)phenyl)methanol (198 mg, 454 μmol) was obtained as a gray solid, which was used directly in the next step. MS: m/z=437.3 [M+H]⁺. ¹H NMR (400 MHz, Dimethylsulfoxide-d₆) δ 9.14-9.09 (m, 1H), 8.32-8.23 (m, 2H), 8.04-7.99 (m, 2H), 7.51-7.43 (m, 4H), 7.36 (d, J=8.0 Hz, 1H), 7.23 (dd, J=7.6, 2.0, Hz, 1H), 6.99 (s, 2H), 6.42 (dd, J=8.0, 4.8 Hz, 1H), 5.54-5.24 (m, 1H), 4.60 (s, 2H), 3.11-2.99 (m, 1H), 1.25 (d, J=6.8 Hz, 6H).

### Step 4: 3-(3-(4-(Chloromethyl)phenyl)-5-(6-isopropylpyridin-3-yl)-3H-imidazo[4,5-b]pyridin-2-yl)pyridin-2-amine

To a solution of (4-(2-(2-aminopyridin-3-yl)-5-(6-isopropylpyridin-3-yl)-3H-imidazo[4,5-b]pyridin-3-yl)phenyl)methanol (198 mg, 454 μmol) in CH₂Cl₂ (8 mL) was added SOCl₂ (162 mg, 1.36 mmol, 98.8 μL). The reaction mixture was stirred at 20° C. for 1 hr. The reaction mixture was concentrated. 3-(3-(4-(Chloromethyl)phenyl)-5-(6-isopropylpyridin-3-yl)-3H-imidazo[4,5-b]pyridin-2-yl)pyridin-2-amine (Intermediate 342, 206 mg, 453 μmol) was obtained as a gray solid, which was used directly in the next step. MS: m/z=455.2, 457.2 [M+H]⁺.

### Intermediate 343: 3-(3-(4-(Chloromethyl)phenyl)-5-(5-cyclopropylpyridin-2-yl)-3H-imidazo[4,5-b]pyridin-2-yl)pyridin-2-amine

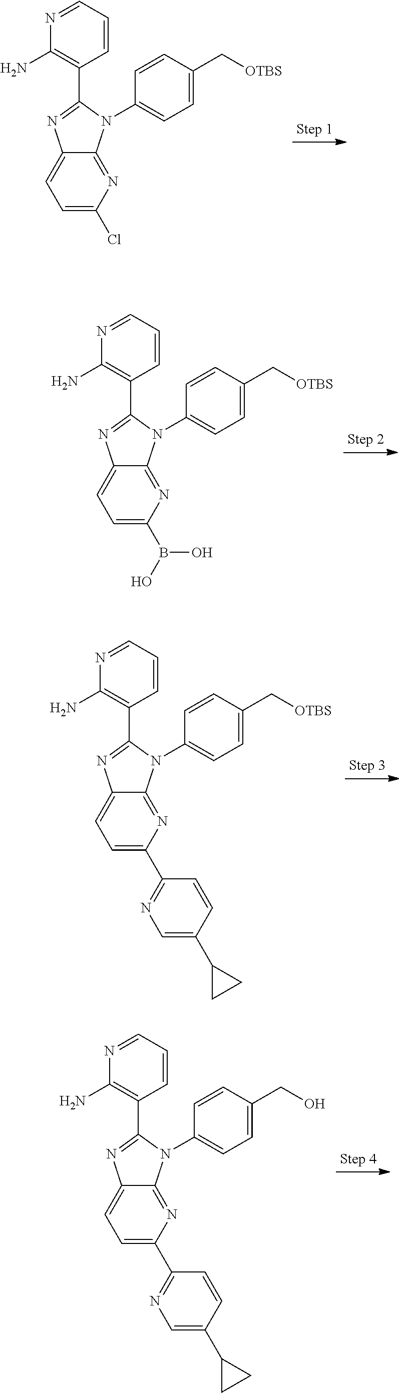 [SMILES: CC(C)(C)[Si](C)(C)OCc1ccc(-n2c(-c3cccnc3N)nc3ccc(Cl)nc32)cc1 ; CC(C)(C)[Si](C)(C)OCc1ccc(-n2c(-c3cccnc3N)nc3ccc(B(O)O)nc32)cc1 ; CC(C)(C)[Si](C)(C)OCc1ccc(-n2c(-c3cccnc3N)nc3ccc(-c4ccc(C5CC5)cn4)nc32)cc1 ; Nc1ncccc1-c1nc2ccc(-c3ccc(C4CC4)cn3)nc2n1-c1ccc(CO)cc1]

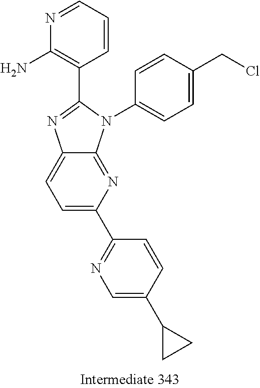 [SMILES: Nc1ncccc1-c1nc2ccc(-c3ccc(C4CC4)cn3)nc2n1-c1ccc(CCl)cc1]

### Step 1: (2-(2-Aminopyridin-3-yl)-3-(4-(((tert-butyldimethylsilyl)oxy)methyl)phenyl)-3H-imidazo[4,5-b]pyridin-5-yl)boronic acid

To a solution of 3-(3-(4-(((tert-butyldimethylsilyl)oxy)methyl)phenyl)-5-chloro-3H-imidazo[4,5-b]pyridin-2-yl)pyridin-2-amine (refer to Intermediate 117 for detail procedures, 500 mg, 1.07 mmol) in 1,4-dioxane (6 mL) were added 4,4,4′,4′,5,5,5′,5′-octamethyl-2,2′-bi(1,3,2-dioxaborolane) (545 mg, 2.15 mmol), KOAc (316 mg, 3.22 mmol), Pd(dppf)Cl₂ (78.5 mg, 107 μmol). The mixture was stirred at 100° C. for 12 hr under N₂. (2-(2-Aminopyridin-3-yl)-3-(4-(((tert-butyldimethylsilyl)oxy)methyl)phenyl)-3H-imidazo[4,5-b]pyridin-5-yl)boronic acid was obtained as a black liquid, which was used in the next step without work-up and purification. MS: m/z=476.3 [M+H]⁺.

### Step 2: 3-(3-(4-(((Tert-butyldimethylsilyl)oxy)methyl)phenyl)-5-(5-cyclopropylpyridin-2-yl)-3H-imidazo[4,5-b]pyridin-2-yl)pyridin-2-amine

To a solution of (2-(2-aminopyridin-3-yl)-3-(4-(((tert-butyldimethylsilyl)oxy)methyl)phenyl)-3H-imidazo[4,5-b]pyridin-5-yl)boronic acid (510 mg, 1.07 mmol) in 1,4-dioxane (10 mL) and H₂O (2 mL) were added 2-bromo-5-cyclopropyl-pyridine (255 mg, 1.29 mmol), Cs₂CO₃ (1.05 g, 3.22 mmol), Pd(dppf)Cl₂ (78.5 mg, 107 μmol). The mixture was stirred at 100° C. for 2 hr under N₂. The mixture was diluted with H₂O (50 mL) and extracted with EtOAc (20 mL×3). The combined organic layers were washed with brine (50 mL), dried over anhydrous Na₂SO₄, filtered and concentrated. After purified by silica gel flash chromatography (0-30% EtOAc in petroleum ether), 3-(3-(4-(((tert-butyldimethylsilyl)oxy)methyl)phenyl)-5-(5-cyclopropylpyridin-2-yl)-3H-imidazo[4,5-b]pyridin-2-yl)pyridin-2-amine (383 mg, yield: 65% for two steps) was obtained as a light yellow solid. MS: m/z=549.3 [M+H]⁺. ¹H NMR (400 MHz, Chloroform-d) δ 8.47-8.44 (m, 2H), 8.19 (d, J=8.4 Hz, 1H), 8.15 (d, J=8.8 Hz, 1H), 8.06 (dd, J=4.8, 2 Hz, 1H), 7.52-7.48 (m, 2H), 7.43-7.38 (m, 2H), 7.33 (dd, J=8, 2 Hz, 1H), 7.13 (dd, J=7.6, 1.6 Hz, 1H), 6.65 (br s, 2H), 6.37 (dd, J=7.6, 4.8 Hz, 1H), 4.87 (s, 2H), 1.90-1.97 (m, 1H), 1.07-1.03 (m, 2H), 0.98 (s, 9H), 0.78-0.74 (m, 2H), 0.16 (s, 6H).

### Step 3: (4-(2-(2-Aminopyridin-3-yl)-5-(5-cyclopropylpyridin-2-yl)-3H-imidazo[4,5-b]pyridin-3-yl)phenyl)methanol

To a solution of 3-(3-(4-(((tert-butyldimethylsilyl)oxy)methyl)phenyl)-5-(5-cyclopropylpyridin-2-yl)-3H-imidazo[4,5-b]pyridin-2-yl)pyridin-2-amine (363 mg, 662 μmol) in THF (4 mL) was added TBAF (1 M in THF, 992 μL). The mixture was stirred at 25° C. for 1 hr under N₂. The mixture was diluted with H₂O (20 mL) and extracted with EtOAc (10 mL×3). The combined organic layers were washed with brine (20 mL), dried over anhydrous Na₂SO₄, filtered and concentrated. (4-(2-(2-Aminopyridin-3-yl)-5-(5-cyclopropylpyridin-2-yl)-3H-imidazo[4,5-b]pyridin-3-yl)phenyl)methanol (268 mg) was obtained as a light yellow solid, which was used in the next step without further purification. MS: m/z=435.2. [M+H]⁺.

### Step 4: 3-(3-(4-(Chloromethyl)phenyl)-5-(5-cyclopropylpyridin-2-yl)-3H-imidazo[4,5-b]pyridin-2-yl)pyridin-2-amine

To a solution of (4-(2-(2-aminopyridin-3-yl)-5-(5-cyclopropylpyridin-2-yl)-3H-imidazo[4,5-b]pyridin-3-yl)phenyl)methanol (248 mg, 571 μmol) in CH₂Cl₂ (2 mL) was added SOCl₂ (204 mg, 1.71 mmol). The mixture was stirred at 25° C. for 2 hr under N₂. The reaction mixture was concentrated, 3-(3-(4-(chloromethyl)phenyl)-5-(5-cyclopropylpyridin-2-yl)-3H-imidazo[4,5-b]pyridin-2-yl)pyridin-2-amine (Intermediate 343, 258 mg) was obtained as a light yellow solid, which was used in the next step without further purification. MS: m/z=453.2, 455.1[M+H]⁺.

### Intermediate 344: 3-(3-(4-(Chloromethyl)phenyl)-5-(6-(difluoromethyl)pyridin-3-yl)-3H-imidazo[4,5-b]pyridin-2-yl)pyridin-2-amine

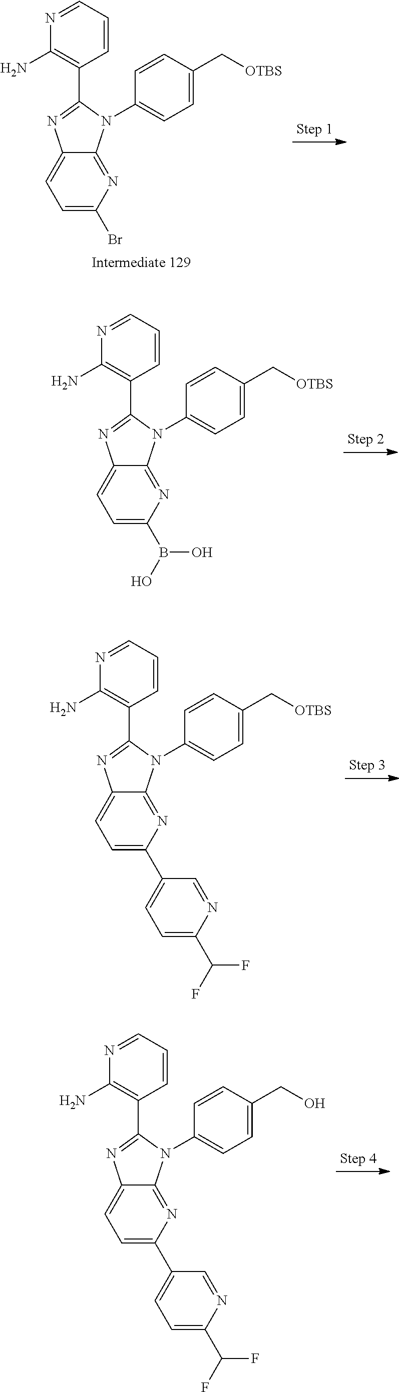 [SMILES: CC(C)(C)[Si](C)(C)OCc1ccc(-n2c(-c3cccnc3N)nc3ccc(Br)nc32)cc1 ; CC(C)(C)[Si](C)(C)OCc1ccc(-n2c(-c3cccnc3N)nc3ccc(B(O)O)nc32)cc1 ; CC(C)(C)[Si](C)(C)OCc1ccc(-n2c(-c3cccnc3N)nc3ccc(-c4ccc(C(F)F)nc4)nc32)cc1 ; Nc1ncccc1-c1nc2ccc(-c3ccc(C(F)F)nc3)nc2n1-c1ccc(CO)cc1]

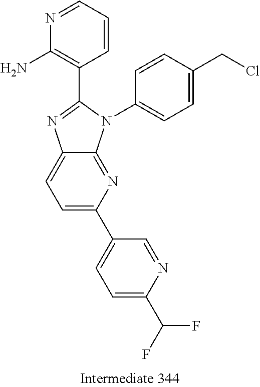 [SMILES: Nc1ncccc1-c1nc2ccc(-c3ccc(C(F)F)nc3)nc2n1-c1ccc(CCl)cc1]

### Step 1; (2-(2-Aminopyridin-3-yl)-3-(4-(((tert-butyldimethylsilyl)oxy)methyl)phenyl)-3H-imidazo[4,5-b]pyridin-5-yl)boronic acid

A mixture of Intermediate 129 (1 g, 1.96 mmol), 4,4,4′,4′,5,5,5′,5′-octamethyl-2,2′-bi(1,3,2-dioxaborolane) (995 mg, 3.92 mmol), KOAc (577 mg, 5.88 mmol) and Pd(dppf)Cl₂ (143 mg, 196 mmol) in 1,4-dioxane (5 mL) was degassed, purged with N₂ three times, and stirred at 90° C. for 16 hr under N₂ atmosphere. (2-(2-Aminopyridin-3-yl)-3-(4-(((tert-butyldimethylsilyl)oxy)methyl)phenyl)-3H-imidazo[4,5-b]pyridin-5-yl)boronic acid was obtained as a black liquid, which was used in the next step without work-up and purification. MS: m/z=476.1 [M+H]⁺.

### Step 2: 3-(3-(4-(((tert-Butyldimethylsilyl)oxy)methyl)phenyl)-5-(6-(difluoromethyl)pyridin-3-yl)-3H-imidazo[4,5-b]pyridin-2-yl)pyridin-2-amine

A mixture of (2-(2-aminopyridin-3-yl)-3-(4-(((tert-butyldimethylsilyl)oxy)methyl)phenyl)-3H-imidazo[4,5-b]pyridin-5-yl)boronic acid (465 mg, 978 μmol), 5-bromo-2-(difluoromethyl)pyridine (224 mg, 1.08 mmol), Pd(dppf)Cl₂ (71.6 mg, 97.8 μmol) and Cs₂CO₃ (956 mg, 2.93 mmol) in 1,4-dioxane (5 mL) and H₂O (1 mL) was degassed, purged with N₂ three times, and stirred at 80° C. for 16 hr under N₂ atmosphere. The reaction mixture was poured into H₂O (20 mL) and extracted with EtOAc (30 mL×3). The combined organic layers were dried over anhydrous Na₂SO₄, filtered and concentrated under reduced pressure. After purified by silica gel flash chromatography (Eluent of 0%˜30% EtOAc in petroleum ether), 3-(3-(4-(((tert-butyldimethylsilyl)oxy)methyl)phenyl)-5-(6-(difluoromethyl)pyridin-3-yl)-3H-imidazo[4,5-b]pyridin-2-yl)pyridin-2-amine (402 mg, yield. 74% for two steps) was obtained as a brown solid. MS: m/z=559.2 [M+H]⁺. ¹H NMR (400 MHz, Dimethysulfoxide-d₆) δ 9.32 (s, 1H), 8.64-8.46 (m, 1H), 8.36 (d, J=8.4 Hz, 1H), 8.15 (d, J=8.4 Hz, 1H), 8.05-7.92 (m, 1H), 7.79 (d, J=8.0 Hz, 1H), 7.52-7.48 (m, 4H), 7.28-7.17 (m, 1H), 7.16-6.83 (m, 3H), 6.40 (dd, J=7.6, 4.8 Hz, 1H), 4.83 (s, 2H), 0.93 (s, 9H), 0.11 (s, 6H). ¹⁹F NMR (400 MHz, Dimethysulfoxide-d₆) δ −115.381.

### Step 3: (4-(2-(2-Aminopyridin-3-yl)-5-(6-(difluoromethyl)pyridin-3-yl)-3H-imidazo[4,5-b]pyridin-3-yl)phenyl)methanol

To a solution of 3-(3-(4-(((tert-butyldimethylsilyl)oxy)methyl)phenyl)-5-(6-(difluoromethyl)pyridin-3-yl)-3H-imidazo[4,5-b]pyridin-2-yl)pyridin-2-amine (402 mg, 720 μmol) in THF (5 mL) was added TBAF (2 mL, 1 M in THF). The mixture was stirred at 25° C. for 1 hr. The reaction mixture was quenched with H₂O (10 mL) and extracted with EtOAc (20 mL×3). The combined organic layers were washed with brine (20 mL×2), dried over Na₂SO₄, filtered and concentrated under reduced pressure. (4-(2-(2-Aminopyridin-3-yl)-5-(6-(difluoromethyl)pyridin-3-yl)-3H-imidazo[4,5-b]pyridin-3-yl)phenyl)methanol (365 mg) was obtained as a brown solid. MS: m/z=445.1 [M+H]⁺.

### Step 4: 3-(3-(4-(Chloromethyl)phenyl)-5-(6-(difluoromethyl)pyridin-3-yl)-3H-imidazo[4,5-b]pyridin-2-yl)pyridin-2-amine

To a solution of (4-(2-(2-aminopyridin-3-yl)-5-(6-(difluoromethyl)pyridin-3-yl)-3H-imidazo[4,5-b]pyridin-3-yl)phenyl)methanol (365 mg, 821 μmol) in CH₂Cl₂ (5 mL) was added SOCl₂ (2 mL). The mixture was stirred at 40° C. for 2 hr. The reaction was concentrated under reduced pressure to give 3-(3-(4-(chloromethyl)phenyl)-5-(6-(difluoromethyl)pyridin-3-yl)-3H-imidazo[4,5-b]pyridin-2-yl)pyridin-2-amine (Intermediate 344, 405 mg, HCl salt) as a brown solid. MS: m/z=463.2, 465.2 [M+H]⁺.

### Intermediate 345: 3-(3-(4-(Chloromethyl)phenyl)-5-(6-(trifluoromethyl)pyridin-3-yl)-3H-imidazo[4,5-b]pyridin-2-yl)pyridin-2-amine

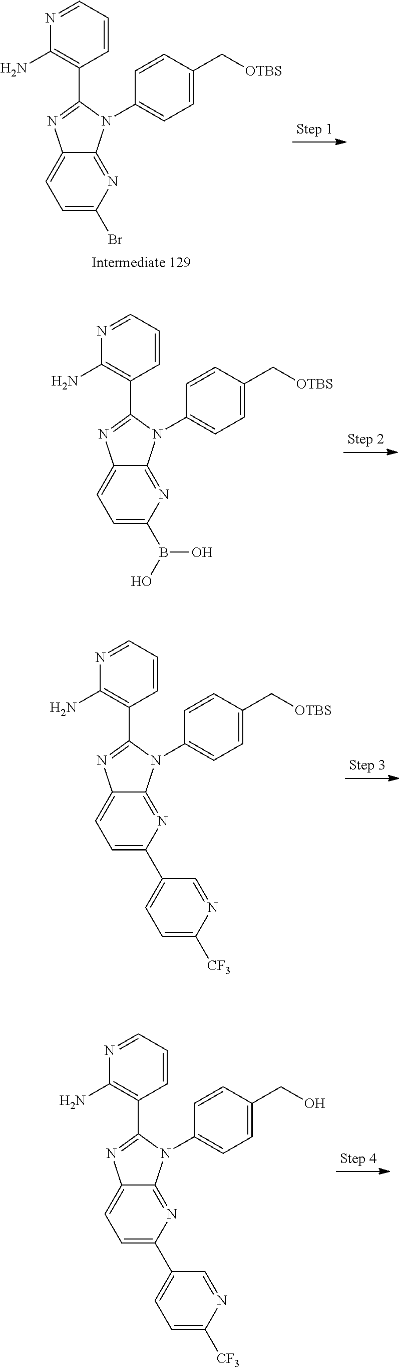 [SMILES: CC(C)(C)[Si](C)(C)OCc1ccc(-n2c(-c3cccnc3N)nc3ccc(Br)nc32)cc1 ; CC(C)(C)[Si](C)(C)OCc1ccc(-n2c(-c3cccnc3N)nc3ccc(B(O)O)nc32)cc1 ; CC(C)(C)[Si](C)(C)OCc1ccc(-n2c(-c3cccnc3N)nc3ccc(-c4ccc(C(F)(F)F)nc4)nc32)cc1 ; Nc1ncccc1-c1nc2ccc(-c3ccc(C(F)(F)F)nc3)nc2n1-c1ccc(CO)cc1]

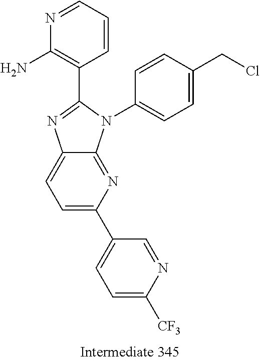 [SMILES: Nc1ncccc1-c1nc2ccc(-c3ccc(C(F)(F)F)nc3)nc2n1-c1ccc(CCl)cc1]

### Step 1: (2-(2-Aminopyridin-3-yl)-3-(4-(((tert-butyldimethylsilyl)oxy)methyl)phenyl)-3H-imidazo[4,5-b]pyridin-5-yl)boronic acid

A mixture of Intermediate 129 (1 g, 1.96 mmol), 4,4,4′,4′,5,5,5′,5′-octamethyl-2,2′-bi(1,3,2-dioxaborolane) (995 mg, 3.92 mmol), KOAc (577 mg, 5.88 mmol) and Pd(dppf)Cl₂ (143 mg, 196 mmol) in 1,4-dioxane (5 mL) was degassed, purged with N₂ three times, and stirred at 90° C. for 16 hr under N₂ atmosphere. (2-(2-Aminopyridin-3-yl)-3-(4-(((tert-butyldimethylsilyl)oxy)methyl)phenyl)-3H-imidazo[4,5-h]pyridin-5-yl)boronic acid was obtained as a black liquid, which was used in the next step without work-up and purification. MS: m/z=476.1 [M+H]⁺.

### Step 2: 3-(3-(4-(((Tert-butyldimethylsilyl)oxy)methyl)phenyl)-5-(6-(trifluoromethyl)pyridin-3-yl)-3H-imidazo[4,5-b]pyridin-2-yl)pyridin-2-amine

A mixture of (2-(2-aminopyridin-3-yl)-3-(4-(((tert-butyldimethylsilyl)oxy)methyl)phenyl)-3H-imidazo[4,5-b]pyridin-5-yl)boronic acid (465 mg, 978 μmol), 5-bromo-2-(trifluoromethyl)pyridine (265 mg, 1.17 mmol), Pd(dppf)Cl₂ (71.6 mg, 97.8 μmol) and Cs₂CO₃ (956 mg, 2.93 mmol) in 1,4-dioxane (5 mL) and H₂O (1 mL) was degassed, purged with N₂ three times, and stirred at 80° C. for 16 hr under N₂ atmosphere. The reaction mixture was poured into H₂O (20 mL) and extracted with EtOAc (20 mL×3). The combined organic layers were dried over anhydrous Na₂SO₄, filtered and concentrated under reduced pressure. After purified by silica gel flash chromatography (Eluent of 0%˜27% EtOAc in petroleum ether), 3-(3-(4-(((tert-butyldimethylsilyl)oxy)methyl)phenyl)-5-(6-(trifluoromethyl)pyridin-3-yl)-3H-imidazo[4,5-b]pyridin-2-yl)pyridin-2-amine (365 mg, yield: 58% for two steps) was obtained as a brown solid. MS: m/z=577.2 [M+H]⁺. ¹H NMR (400 MHz, Dimethysulfoxide-d₆) δ 9.40 (s, 1H), 8.76-8.51 (m, 1H), 8.38 (d, J=8.4 Hz, 1H), 8.20 (d, J=8.4 Hz, 1H), 8.09-7.90 (m, 2H), 7.52-7.48 (m, 4H), 7.28-7.15 (m, 1H), 7.03 (br s, 2H), 6.40 (dd, J=7.6, 5.6 Hz, 1H), 4.83 (s, 2H), 0.93 (s, 9H), 0.11 (s, 6H). ¹⁹F NMR (400 MHz, Dimethysulfoxide-d₆) δ −66.315.

### Step 3: (4-(2-(2-Aminopyridin-3-yl)-5-(6-(trifluoromethyl)pyridin-3-yl)-3H-imidazo[4,5-b]pyridin-3-yl)phenyl)methanol

To a solution of 3-(3-(4-(((tert-butyldimethylsilyl)oxy)methyl)phenyl)-5-(6-(trifluoromethyl)pyridin-3-yl)-3H-imidazo[4,5-b]pyridin-2-yl)pyridin-2-amine (326 mg, 565 μmol) in THF (3 mL) was added TBAF (1 mL, 1 M in THF). The mixture was stirred at 25° C. for 0.5 hr. The reaction mixture was quenched with H₂O (10 mL) and extracted with EtOAc (20 mL×3). The combined organic layers were washed with brine (20 mL×2), dried over Na₂SO₄, filtered and concentrated under reduced pressure to give (4-(2-(2-aminopyridin-3-yl)-5-(6-(trifluoromethyl)pyridin-3-yl)-3H-imidazo[4,5-b]pyridin-3-yl)phenyl)methanol (263 mg) as a brown solid. MS: m/z=463.2 [M+H]⁺.

### Step 4: 3-(3-(4-(Chloromethyl)phenyl)-5-(6-(trifluoromethyl)pyridin-3-yl)-3H-imidazo[4,5-b]pyridin-2-yl)pyridin-2-amine

To a solution of (4-(2-(2-aminopyridin-3-yl)-5-(6-(trifluoromethyl)pyridin-3-yl)-3H-imidazo[4,5-b]pyridin-3-yl)phenyl)methanol (263 mg, 569 μmol) in CH₂Cl₂ (2 mL) was added SOCl₂ (1 mL). The mixture was stirred at 25° C. for 0.5 hr. The reaction was concentrated under reduced pressure to give 3-(3-(4-(chloromethyl)phenyl)-5-(6-(trifluoromethyl)pyridin-3-yl)-3H-imidazo[4,5-b]pyridin-2-yl)pyridin-2-amine (Intermediate 345, 276 mg, HCl salt) as a yellow solid. MS: m/z=481.1, 483.1 [M+H]⁺.

### Intermediate 346: 3-(3-(4-(Chloromethyl)phenyl)-5-(5-ethylpyridin-2-yl)-3H-imidazo[4,5-b]pyridin-2-yl)pyridin-2-amine

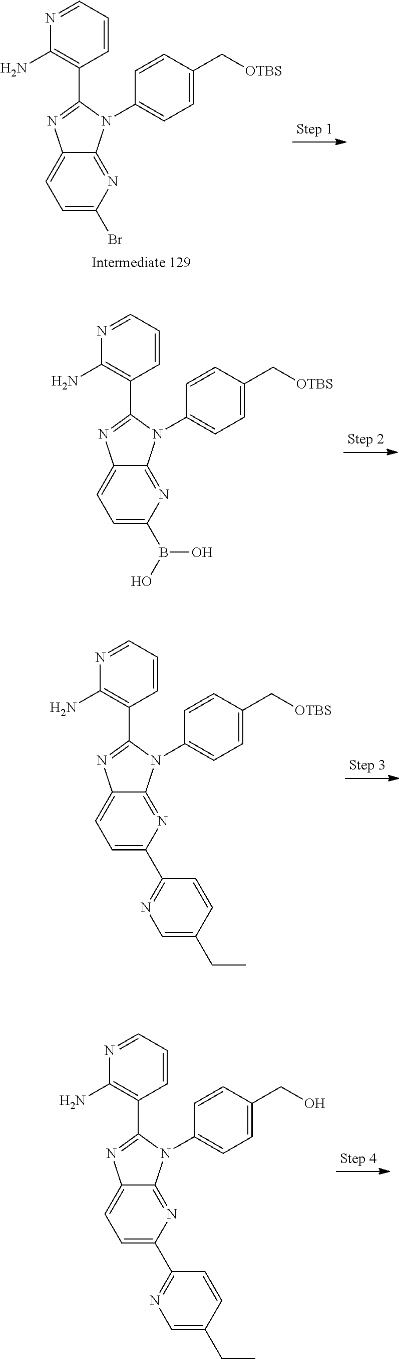 [SMILES: CC(C)(C)[Si](C)(C)OCc1ccc(-n2c(-c3cccnc3N)nc3ccc(Br)nc32)cc1 ; CC(C)(C)[Si](C)(C)OCc1ccc(-n2c(-c3cccnc3N)nc3ccc(B(O)O)nc32)cc1 ; CCc1ccc(-c2ccc3nc(-c4cccnc4N)n(-c4ccc(CO[Si](C)(C)C(C)(C)C)cc4)c3n2)nc1 ; CCc1ccc(-c2ccc3nc(-c4cccnc4N)n(-c4ccc(CO)cc4)c3n2)nc1]

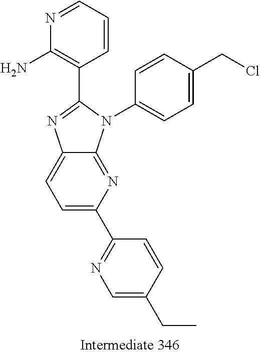 [SMILES: CCc1ccc(-c2ccc3nc(-c4cccnc4N)n(-c4ccc(CCl)cc4)c3n2)nc1]

### Step 1: (2-(2-Aminopyridin-3-yl)-3-(4-(((tert-butyldimethylsilyl)oxy)methyl)phenyl)-3H-imidazo[4,5-b]pyridin-5-yl)boronic acid

A mixture of Intermediate 129 (600 mg, 1.18 mmol), 4,4,4′,4′,5,5,5′,5′-octamethyl-2,2′-bi(1,3,2-dioxaborolane) (596 mg, 2.35 mmol), KOAc (346 mg, 3.53 mmol) and Pd(dppf)Cl₂ (86.0 mg, 118 μmol) in 1,4-dioxane (6 mL) was degassed, purged with N₂ three times, and stirred at 90° C. for 2 hr under N₂ atmosphere. (2-(2-Aminopyridin-3-yl)-3-(4-(((tert-butyldimethylsilyl)oxy)methyl)phenyl)-3H-imidazo[4,5-b]pyridin-5-yl)boronic acid was obtained as a black liquid, which was used in the next step without work-up and purification. MS: m/z=476.1 [M+H]⁺.

### Step 2: 3-(3-(4-(((tert-Butyldimethylsilyl)oxy)methyl)phenyl)-5-(5-ethylpyridin-2-yl)-3H-imidazo[4,5-b]pyridin-2-yl)pyridin-2-amine

A mixture of (2-(2-aminopyridin-3-yl)-3-(4-(((tert-butyldimethylsilyl)oxy)methyl)phenyl)-3H-imidazo[4,5-b]pyridin-5-yl)boronic acid (558 mg, 1.17 mmol), 2-bromo-5-ethylpyridine (262 mg, 1.41 mmol), Pd(dppf)Cl₂ (85.9 mg, 117 μmol) and Cs₂CO₃ (1.15 g, 3.52 mmol) in 1,4-dioxane (6 mL) and H₂O (1 mL) was degassed, purged with N₂ three times, and stirred at 80° C. for 5 hr under N₂ atmosphere. The reaction mixture was quenched with H₂O (20 mL) and extracted with CH₂Cl₂ (25 mL×2). The combined organic layers were washed with brine (30 mL), dried over Na₂SO₄, filtered and concentrated under reduced pressure. After purified by silica gel flash chromatography (Eluent of 15%˜32% EtOAc in petroleum ether), 3-(3-(4-(((tert-butyldimethylsilyl)oxy)methyl)phenyl)-5-(5-ethylpyridin-2-yl)-3H-imidazo[4,5-b]pyridin-2-yl)pyridin-2-amine (320 mg, yield: 43% for two steps) was obtained as a yellow solid. MS: m/z=537.3 [M+H]⁺. ¹H NMR (400 MHz, Chloroform-d) δ 8.54-8.46 (m, 2H), 8.23 (d, J=8.0 Hz, 1H), 8.16 (d, J=8.4 Hz, 1H), 8.08-8.04 (m, 1H), 7.58-7.54 (m, 1H), 7.52-7.48 (m, 2H), 7.44-7.38 (m, 2H), 7.16-7.11 (m, 1H), 6.68 (br s, 2H), 6.39-6.34 (m, 1H), 4.87 (s, 2H), 2.69 (q, J=7.6 Hz, 2H), 1.29-1.25 (m, 3H), 0.98 (s, 9H), 0.16 (s, 6H).

### Step 3: (4-(2-(2-Aminopyridin-3-yl)-5-(5-ethylpyridin-2-yl)-3H-imidazo[4,5-b]pyridin-3-yl)phenyl)methanol

To a solution of 3-(3-(4-(((tert-butyldimethylsilyl)oxy)methyl)phenyl)-5-(5-ethylpyridin-2-yl)-3H-imidazo[4,5-b]pyridin-2-yl)pyridin-2-amine (320 mg, 596 μmol) in THF (5 mL) was added TBAF (1.79 mL, 1 M in THF). The mixture was stirred at 25° C. for 1 hr. The reaction mixture was quenched with H₂O (20 mL) at 25° C. and extracted with CH₂Cl₂ (25 mL×2). The combined organic layers were washed with brine (50 mL×5), dried over Na₂SO₄, filtered and concentrated under reduced pressure. After purified by triturated with EtOAc (10 mL) at 25° C. for 10 min, (4-(2-(2-aminopyridin-3-yl)-5-(5-ethylpyridin-2-yl)-3H-imidazo[4,5-b]pyridin-3-yl)phenyl)methanol (210 mg, yield: 79%) was obtained as a yellow solid. MS: m/z=423.0 [M+H]⁺. ¹H NMR (400 MHz, Dimethylsulfoxide-d₆) δ 8.53 (d, J=2.0 Hz, 1H), 8.43 (d, J=8.4 Hz, 1H), 8.28 (d, J=8.4 Hz, 1H), 8.10 (d, J=8.4 Hz, 1H), 8.00 (dd, J=4.8, 2.0 Hz, 1H), 7.74 (dd, J=8.4, 2.4 Hz, 1H), 7.53-7.49 (m, 2H), 7.47-7.44 (m, 2H), 7.23 (dd, J=7.6, 2.0 Hz, 1H), 6.95 (br s, 2H), 6.42 (dd, J=8.0, 4.8 Hz, 1H), 5.37 (br t, 0.1=6.0 Hz, 1H), 4.61 (d, J=5.6 Hz, 2H), 2.66 (q, J=7.6 Hz, 2H), 1.21 (t, J=7.6 Hz, 3H).

### Step 4: 3-(3-(4-(Chloromethyl)phenyl)-5-(5-ethylpyridin-2-yl)-3H-imidazo[4,5-b]pyridin-2-yl)pyridin-2-amine

To a solution of (4-(2-(2-aminopyridin-3-yl)-5-(5-ethylpyridin-2-yl)-3H-imidazo[4,5-b]pyridin-3-yl)phenyl)methanol (210 mg, 497 μmol) in CH₂Cl₂ (5 mL) was added SOCl₂ (177 mg, 1.49 mmol). The mixture was stirred at 40° C. for 0.5 hr. The reaction was concentrated under reduced pressure to give 3-(3-(4-(chloromethyl)phenyl)-5-(5-ethylpyridin-2-yl)-3H-imidazo[4,5-b]pyridin-2-yl)pyridin-2-amine (Intermediate 346, 237 mg, HCl salt) as a yellow solid. MS: m/z=441.1, 443.1[M+H]⁺. ¹H NMR (400 MHz, Dimethylsulfoxide-d₆) δ 8.60 (s, 1H), 8.55-8.47 (m, 1H), 8.46-8.27 (m, 2H), 8.23 (d, J=8.0 Hz, 1H), 8.13 (d, J=5.6 Hz, 1H), 7.99-7.87 (m, 2H), 7.71-7.58 (m, 4H), 6.94-6.88 (m, 1H), 4.88 (s, 2H), 2.75-2.69 (m, 2H), 1.23 (t, J=7.6 Hz, 3H).

### Intermediate 347: 2-Bromo-5-(fluoromethyl)pyridine

To a solution of (6-bromopyridin-3-yl)methanol (1 g, 5.32 mmol) in CH₂Cl₂ (25 mL) was added DAST (857 mg, 5.32 mmol) at 0° C. The mixture was stirred at 0° C. for 1 hr. The reaction mixture was quenched with NaHCO₃ (30 mL) at 0° C. and extracted with CH₂Cl₂ (50 mL×2). The combined organic layers were washed with brine (50 mL), dried over Na₂SO₄, filtered and concentrated under reduced pressure. After purified by silica gel flash chromatography (Eluent of 0%˜5% EtOAc in petroleum ether), 2-bromo-5-(fluoromethyl)pyridine (Intermediate 347, 300 mg, yield: 28%) was obtained as a light yellow oil. MS: m/z=189.8, 191.8 [M+H]⁺. ¹H NMR (400 MHz, Chloroform-d) δ 8.39 (s, 1H), 7.71-7.47 (m, 2H), 5.37 (d, J=47.2 Hz, 2H). ¹⁹F NMR (400 MHz, Chloroform-d) 6-211.43.

### Intermediate 348: 3-(3-(4-(Chloromethyl)phenyl)-5-(5-(fluoromethyl)pyridin-2-yl)-3H-imidazo[4,5-b]pyridin-2-yl)pyridin-2-amine

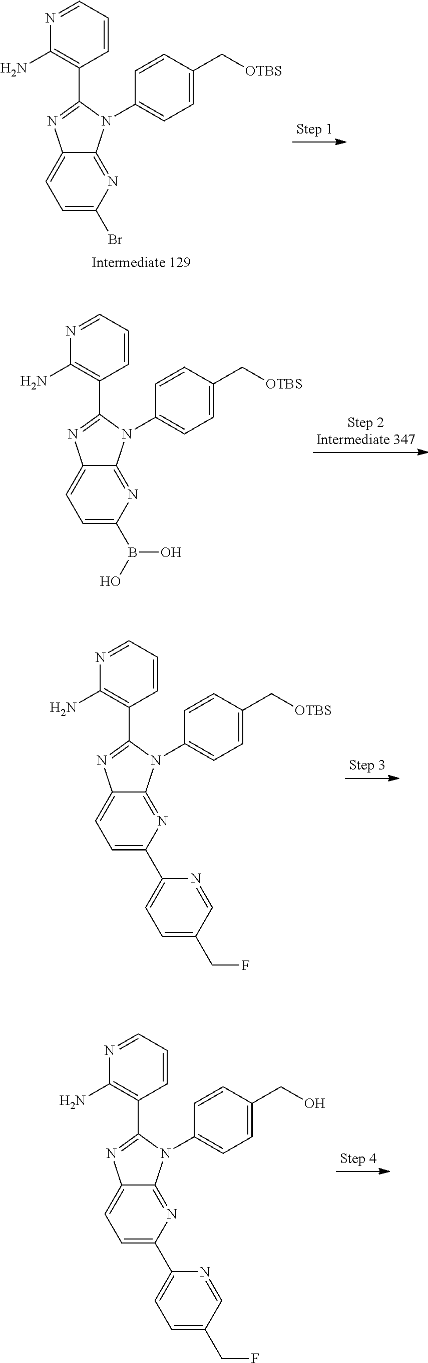 [SMILES: CC(C)(C)[Si](C)(C)OCc1ccc(-n2c(-c3cccnc3N)nc3ccc(Br)nc32)cc1 ; CC(C)(C)[Si](C)(C)OCc1ccc(-n2c(-c3cccnc3N)nc3ccc(B(O)O)nc32)cc1 ; CC(C)(C)[Si](C)(C)OCc1ccc(-n2c(-c3cccnc3N)nc3ccc(-c4ccc(CF)cn4)nc32)cc1 ; Nc1ncccc1-c1nc2ccc(-c3ccc(CF)cn3)nc2n1-c1ccc(CO)cc1]

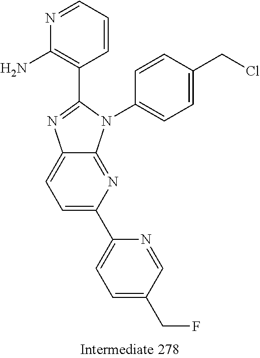 [SMILES: Nc1ncccc1-c1nc2ccc(-c3ccc(CF)cn3)nc2n1-c1ccc(CCl)cc1]

### Step 1: (2-(2-Aminopyridin-3-yl)-3-(4-(((tert-butyldimethylsilyl)oxy)methyl)phenyl)-3H-imidazo[4,5-b]pyridin-5-yl)boronic acid

A mixture of Intermediate 129 (550 mg, 1.08 mmol), 4,4,4′,4′,5,5,5′,5′-octamethyl-2,2′-bi(1,3,2-dioxaborolane) (547 mg, 2.15 mmol), KOAc (317 mg, 3.23 mmol) and Pd(dppf)Cl₂ (78.8 mg, 108 μmol) in 1,4-dioxane (6 mL) was degassed, purged with N₂ three times, and stirred at 90° C. for 2 hr under N₂ atmosphere. (2-(2-Aminopyridin-3-yl)-3-(4-(((tert-butyldimethylsilyl)oxy)methyl)phenyl)-3H-imidazo[4,5-b]pyridin-5-yl)boronic acid was obtained as a black liquid, the mixture was used for next step directly and without work-up and purification. MS: m/z=476.0 [M+H]⁺.

### Step 2: 3-(3-(4-(((Tert-butyldimethylsilyl)oxy)methyl)phenyl)-5-(5-(fluoromethyl)pyridin-2-yl)-3H-imidazo[4,5-b]pyridin-2-yl)pyridin-2-amine

A mixture of (2-(2-aminopyridin-3-yl)-3-(4-(((tert-butyldimethylsilyl)oxy)methyl)phenyl)-3H-imidazo[4,5-b]pyridin-5-yl)boronic acid (512 mg, 1.08 mmol), Intermediate 347 (286 mg, 1.51 mmol), Cs₂CO₃ (1.05 g, 3.23 mmol) and Pd(dppf)Cl₂ (78.8 mg, 108 μmol) in 1,4-dioxane (6 mL) and H₂O (1 mL) was degassed, purged with N₂ three times, and stirred at 80° C. for 16 hr under N₂ atmosphere. The reaction mixture was quenched with H₂O (20 mL) and extracted with CH₂Cl₂ (25 mL×2). The combined organic layers were washed with brine (50 mL), dried over Na₂SO₄, filtered and concentrated under reduced pressure. After purified by silica gel flash chromatography (Eluent of 10%˜32% EtOAc in petroleum ether), 3-(3-(4-(((tert-butyldimethylsilyl)oxy)methyl)phenyl)-5-(5-(fluoromethyl)pyridin-2-yl)-3H-imidazo[4,5-b]pyridin-2-yl)pyridin-2-amine (310 mg, yield: 51% for two steps) was obtained as a yellow solid. MS: m/z=541.3 [M+H]⁺. ¹H NMR (400 MHz, Dimethylsulfoxide-d₆) δ 8.73 (s, 1H), 8.49 (d, J=8.4 Hz, 1H), 8.32 (d, J=8.8 Hz, 1H), 8.21 (d, J=8.0 Hz, 1H), 8.01 (d, J=2.8 Hz, 1H), 7.95 (d, J=8.4 Hz, 1H), 7.52-7.48 (m, 4H), 7.23-7.19 (m, 1H), 7.00 (br s, 2H), 6.39 (dd, J=7.6, 5.2 Hz, 1H), 5.54 (d, J=47.6 Hz, 2H), 4.84 (s, 2H), 0.93 (s, 9H), 0.12 (s, 6H). ¹⁹F NMR (400 MHz, Dimethylsulfoxide-d₆) δ −206.805.

### Step 3: (4-(2-(2-Aminopyridin-3-yl)-5-(5-(fluoromethyl)pyridin-2-yl)-3H-imidazo[4,5-b]pyridin-3-yl)phenyl)methanol

To a solution of 3-(3-(4-(((tert-butyldimethylsilyl)oxy)methyl)phenyl)-5-(5-(fluoromethyl)pyridin-2-yl)-3H-imidazo[4,5-b]pyridin-2-yl)pyridin-2-amine (310 mg, 573 μmol) in THF (5 mL) was added TBAF (1.72 mL, 1 M in THF). The mixture was stirred at 25° C. for 1 hr. The reaction mixture was quenched with H₂O (20 mL) at 25° C. and extracted with CH₂Cl₂ (25 mL×2). The combined organic layers were washed with brine (50 mL×5), dried over Na₂SO₄, filtered and concentrated under reduced pressure. (4-(2-(2-Aminopyridin-3-yl)-5-(5-(fluoromethyl)pyridin-2-yl)-3H-imidazo[4,5-b]pyridin-3-yl)phenyl)methanol (140 mg) was obtained as a yellow solid. MS: m/z=427.1 [M+H]⁺. ¹H NMR (400 MHz, Dimethylsulfoxide-d₆) δ 8.73 (s, 1H), 8.49 (d, J=8.0 Hz, 1H), 8.32 (d, J=8.4 Hz, 1H), 8.22 (d, J=8.0 Hz, 1H), 8.01 (dd, J=4.8, 1.6 Hz, 1H), 7.99-7.95 (m, 1H), 7.53-7.46 (m, 4H), 7.24 (dd, J=7.6, 2.0 Hz, 1H), 6.96 (br s, 2H), 6.43 (dd, J=7.6, 4.8 Hz, 1H), 5.53 (d, J=47.6 Hz, 2H), 5.37 (br t, J=6.0 Hz, 1H), 4.62 (d, J=5.6 Hz, 2H). ¹⁹F NMR (400 MHz, Dimethylsulfoxide-d₆) δ −206.75.

### Step 4: 3-(3-(4-(Chloromethyl)phenyl)-5-(5-(fluoromethyl)pyridin-2-yl)-3H-imidazo[4,5-b]pyridin-2-yl)pyridin-2-amine

To a solution of (4-(2-(2-aminopyridin-3-yl)-5-(5-(fluoromethyl)pyridin-2-yl)-3H-imidazo[4,5-b]pyridin-3-yl)phenyl)methanol (140 mg, 328 μmol) in CH₂Cl₂ (5 mL) was added SOCl₂ (117 mg, 985 μmol). The mixture was stirred at 40° C. for 0.5 hr. The reaction was concentrated under reduced pressure to give 3-(3-(4-(chloromethyl)phenyl)-5-(5-(fluoromethyl)pyridin-2-yl)-3H-imidazo[4,5-b]pyridin-2-yl)pyridin-2-amine (Intermediate 348, 158 mg, HCl salt) as a yellow solid. MS: m/z=445.1, 447.1 [M+H]⁺. ¹H NMR (400 MHz, Dimethylsulfoxide-d₆) δ 8.76 (s, 1H), 8.57 (d, J=8.4 Hz, 1H), 8.46-8.35 (m, 2H), 8.28 (d, J=8.4 Hz, 1H), 8.14 (dd, J=6.4, 1.6 Hz, 1H), 8.05-8.00 (m, 1H), 7.94 (dd, J=7.6, 1.2 Hz, 1H), 7.68-7.61 (m, 4H), 6.93 (dd, J=7.2, 6.4 Hz, 1H), 5.55 (d, J=47.6 Hz, 2H), 4.88 (s, 2H). ¹⁹F NMR (400 MHz, Dimethylsulfoxide-d₆) δ −207.28.

### Intermediate 349: 3-(3-(4-(Chloromethyl)phenyl)-5-(5-(difluoromethyl)pyridin-2-yl)-3H-imidazo[4,5-b]pyridin-2-yl)pyridin-2-amine

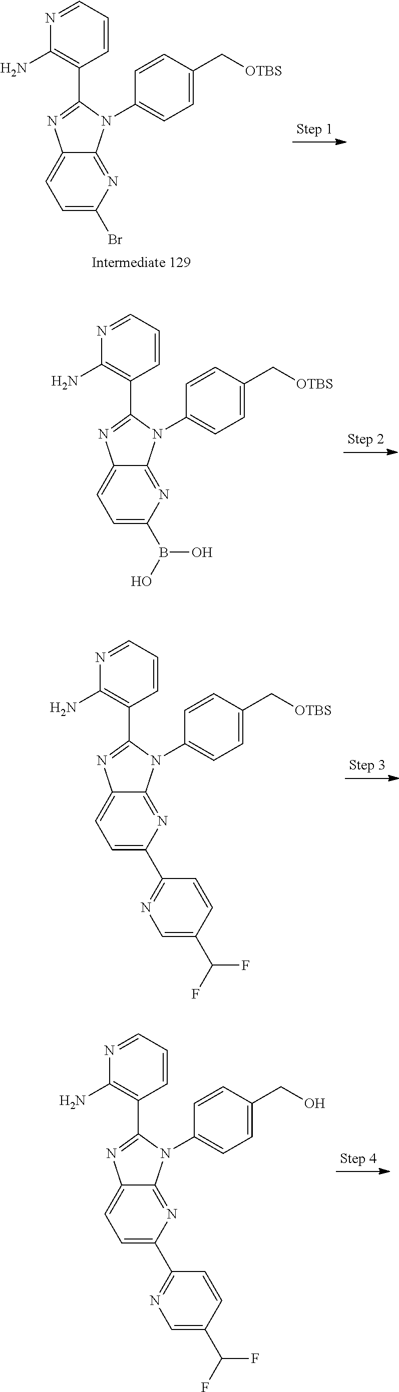 [SMILES: CC(C)(C)[Si](C)(C)OCc1ccc(-n2c(-c3cccnc3N)nc3ccc(Br)nc32)cc1 ; CC(C)(C)[Si](C)(C)OCc1ccc(-n2c(-c3cccnc3N)nc3ccc(B(O)O)nc32)cc1 ; CC(C)(C)[Si](C)(C)OCc1ccc(-n2c(-c3cccnc3N)nc3ccc(-c4ccc(C(F)F)cn4)nc32)cc1 ; Nc1ncccc1-c1nc2ccc(-c3ccc(C(F)F)cn3)nc2n1-c1ccc(CO)cc1]

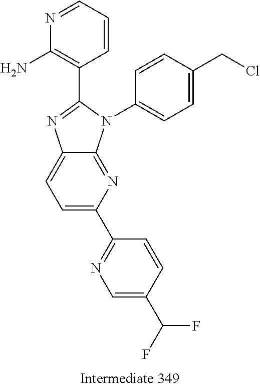 [SMILES: Nc1ncccc1-c1nc2ccc(-c3ccc(C(F)F)cn3)nc2n1-c1ccc(CCl)cc1]

### Step 1: (2-(2-Aminopyridin-3-yl)-3-(4-(((tert-butyldimethylsilyl)oxy)methyl)phenyl)-3H-imidazo[4,5-b]pyridin-5-yl)boronic acid

A mixture of Intermediate 129 (500 mg, 979 μmol), 4,4,4′,4′,5,5,5′,5′-octamethyl-2,2′-bi(1,3,2-dioxaborolane) (498 mg, 1.96 mmol), KOAc (288 mg, 2.94 mmol) and Pd(dppf)Cl₂ (71.7 mg, 97.9 μmol) in 1,4-dioxane (5 mL) was degassed, purged with N₂ three times, and stirred at 90° C. for 8 hr under N₂ atmosphere. (2-(2-Aminopyridin-3-yl)-3-(4-(((tert-butyldimethylsilyl)oxy)methyl)phenyl)-3H-imidazo[4,5-b]pyridin-5-yl)boronic acid was obtained as a black liquid, the mixture was used for next step directly and without work-up and purification. MS: m/z=476.2 [M+H]⁺.

### Step 2: 3-(3-(4-(((tert-Butyldimethylsilyl)oxy)methyl)phenyl)-5-(5-(difluoromethyl)pyridin-2-yl)-3H-imidazo[4,5-b]pyridin-2-yl)pyridin-2-amine

A mixture of (2-(2-aminopyridin-3-yl)-3-(4-(((tert-butyldimethylsilyl)oxy)methyl)phenyl)-3H-imidazo[4,5-b]pyridin-5-yl)boronic acid (465 mg, 978 μmol), 2-bromo-5-(difluoromethyl)pyridine (203 mg, 978 μmol), Pd(dppf)Cl₂ (71.6 mg, 98 μmol) and Cs₂CO₃ (957 mg, 2.93 mmol) in 1,4-dioxane (10 mL) and H₂O (2 mL) was degassed and purged with N₂ three times. The mixture was stirred at 80° C. for 16 hr under N₂ atmosphere. The reaction mixture was quenched with H₂O (20 mL) and extracted with EtOAc (20 mL×3). The combined organic layers were washed with brine (60 mL), dried over Na₂SO₄, filtered and concentrated under reduced pressure. After purified by silica gel flash chromatography (Eluent of 10%˜37% EtOAc in petroleum ether), 3-(3-(4-(((tert-butyldimethylsilyl)oxy)methyl)phenyl)-5-(5-(difluoromethyl)pyridin-2-yl)-3H-imidazo[4,5-b]pyridin-2-yl)pyridin-2-amine (370 mg, yield: 52% for two steps) was obtained as a brown solid. MS: m/z=559.2 [M+H]⁺. ¹H NMR (400 MHz, Dimethylsulfoxide-d₆) δ 8.87 (s, 1H), 8.51 (d, J=8.4 Hz, 1H), 8.38-8.26 (m, 2H), 8.16-8.05 (m, 1H), 8.04-7.95 (m, 1H), 7.58-7.37 (m, 5H), 7.23-7.19 (m, 1H), 7.00 (br s, 2H), 6.42-6.35 (m, 1H), 4.86-4.79 (m, 2H), 0.95-0.91 (m, 9H), 0.14-0.10 (m, 6H). ¹⁹F NMR (400 MHz, Dimethylsulfoxide-d₆) 6-111.254.

### Step 3: (4-(2-(2-Aminopyridin-3-yl)-5-(5-(difluoromethyl)pyridin-2-yl)-3H-imidazo[4,5-b]pyridin-3-yl)phenyl)methanol

To a solution of 3-(3-(4-(((tert-butyldimethylsilyl)oxy)methyl)phenyl)-5-(5-(difluoromethyl)pyridin-2-yl)-3H-imidazo[4,5-b]pyridin-2-yl)pyridin-2-amine (370 mg, 662 μmol) in THF (5 mL) was added TBAF (2 mL, 1 M in THF). The mixture was stirred at 25° C. for 1 hr. The reaction mixture was quenched with H₂O (20 mL) at 25° C. and extracted with EtOAc (20 mL×3). The combined organic layers were washed with brine (60 mL×3), dried over Na₂SO₄, filtered and concentrated under reduced pressure. The crude product was triturated with CH₂Cl₂:petroleum ether=1:10 (6 mL) at 25° C. for 10 min. (4-(2-(2-Aminopyridin-3-yl)-5-(5-(difluoromethyl)pyridin-2-yl)-3H-imidazo[4,5-b]pyridin-3-yl)phenyl)methanol (240 mg, yield: 58%) was obtained as a brown solid. MS: m/z=445.1 [M+H]⁺. ¹H NMR (400 MHz, Dimethylsulfoxide-d₆) δ 8.87 (d, J=0.8 Hz, 1H), 8.51 (d, J=8.4 Hz, 1H), 8.37-8.25 (m, 2H), 8.12 (d, J=8.0 Hz, 1H), 8.05-7.96 (m, 1H), 7.54-7.44 (m, 4H), 7.37-7.33 (m, 0.4H), 7.24 (dd, J=7.6, 2.0 Hz, 1H), 7.20-7.19 (m, 0.4H), 7.07-7.05 (m, 0.2H), 6.99-6.91 (m, 2H), 6.46-6.40 (m, 1H), 5.42-5.33 (m, 1H), 4.65-4.59 (m, 2H). ¹⁹F NMR (400 MHz, Dimethylsulfoxide-d₆) δ −111.245.

### Step 4: 3-(3-(4-(Chloromethyl)phenyl)-5-(5-(difluoromethyl)pyridin-2-yl)-3H-imidazo[4,5-b]pyridin-2-yl)pyridin-2-amine

To a solution of (4-(2-(2-aminopyridin-3-yl)-5-(5-(difluoromethyl)pyridin-2-yl)-3H-imidazo[4,5-b]pyridin-3-yl)phenyl)methanol (240 mg, 540 μmol) in CH₂Cl₂ (5 mL) was added SOCl₂ (118 μL, 1.62 mmol). The mixture was stirred at 40° C. for 0.5 hr. The reaction was concentrated under reduced pressure to give 3-(3-(4-(chloromethyl)phenyl)-5-(5-(difluoromethyl)pyridin-2-yl)-3H-imidazo[4,5-b]pyridin-2-yl)pyridin-2-amine (Intermediate 349, 270 mg, HCl salt) as a brown solid. MS: m/z=463.1, 465.1 [M+H]⁺.

### Example 1: 6-((1-(4-(2-(2-Aminopyridin-3-yl)-3H-imidazo[4,5-b]pyridin-3-yl)benzyl)piperidin-4-yl)oxy)nicotinonitrile

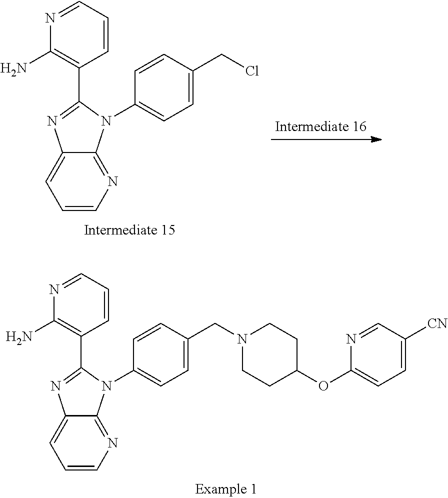 [SMILES: Nc1ncccc1-c1nc2cccnc2n1-c1ccc(CCl)cc1 ; N#Cc1ccc(OC2CCN(Cc3ccc(-n4c(-c5cccnc5N)nc5cccnc54)cc3)CC2)nc1]

To a solution of Intermediate 15 (50 mg, 149 μmol) in DMF (2 mL) was added NaI (3 mg, 14.9 μmol), K₂CO₃ (41 mg, 298 μmol) and Intermediate 16 (36 mg, 179 μmol). The mixture was stirred at 80° C. for 2 hr. The mixture was diluted with 20 mL H₂O and extracted with EtOAc (20 mL×2). The combined organic layers were washed with 20 mL brine, dried over Na₂SO₄, filtered, and concentrated under reduced pressure to give a residue. The crude product was purified by flash chromatography on silica gel (Eluent of 0˜8% MeOH in CH₂Cl₂) to give 6-((1-(4-(2-(2-aminopyridin-3-yl)-3H-imidazo[4,5-b]pyridin-3-yl)benzyl)piperidin-4-yl)oxy)nicotinonitrile (Example 1, 24.1 mg, yield: 32%) as a light yellow solid. MS: m/z=503.1 [M+H]⁺. ¹H NMR (400 MHz, Dimethylsulfoxide-d₆) δ 8.68 (d, J=1.6 Hz, 1H), 8.32 (d, J=4.0 Hz, 1H), 8.20 (d, J=7.6 Hz, 1H), 8.13 (dd, J=8.8, 2.0 Hz, 1H), 7.98 (br d, J=3.2 Hz, 1H), 7.46 (d, J=8.0 Hz, 2H), 7.40-7.37 (m, 3H), 7.16 (d, J=6.4 Hz, 1H), 7.01-6.96 (m, 3H), 6.37 (dd, J=7.6, 4.8 Hz, 1H), 5.15-5.09 (m, 1H), 3.58 (s, 2H), 2.76-2.70 (m, 2H), 2.32-2.25 (m, 2H), 2.03-1.98 (m, 2H), 1.77-1.68 (m, 2H).

### Example 2: 6-((1-(4-(2-(2-Aminopyridin-3-yl)-5-phenyl-3H-imidazo[4,5-b]pyridin-3-yl)benzyl)piperidin-4-yl)oxy)nicotinonitrile

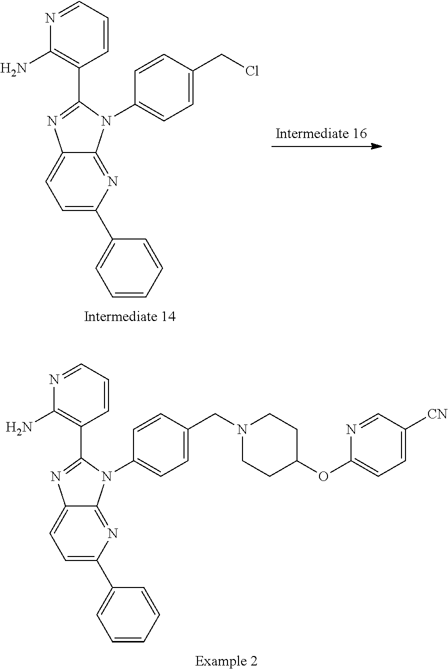 [SMILES: Nc1ncccc1-c1nc2ccc(-c3ccccc3)nc2n1-c1ccc(CCl)cc1 ; N#Cc1ccc(OC2CCN(Cc3ccc(-n4c(-c5cccnc5N)nc5ccc(-c6ccccc6)nc54)cc3)CC2)nc1]

To a solution of Intermediate 14 (200 mg, 485 μmol) and 6-(4-piperidyloxy)pyridine-3-carbonitrile (128 mg, 631 μmol) in DMF (1 mL) was added K₂CO₃ (134 mg, 971 μmol) and NaI (7.28 mg, 48.5 μmol). The mixture was stirred at 80° C. for 2 hr. The reaction mixture was diluted with H₂O (5 mL) and then filtered and concentrated under reduced pressure to give a residue. The residue was purified by silica gel flash chromatography (Eluent of 0˜6% MeOH in CH₂Cl₂), and then purified by prep-TLC (CH₂Cl₂:MeOH=10:1). 6-((1-(4-(2-(2-aminopyridin-3-yl)-5-phenyl-3H-imidazo[4,5-b]pyridin-3-yl)benzyl)piperidin-4-yl)oxy)nicotinonitrile (Example 2, 21.0 mg, yield: 7.5%) was obtained as a light-yellow powder. MS: m/z=579.3 [M+H]⁺. ¹H NMR (400 MHz, Dimethylsulfoxide-d₆) δ 8.68 (d, J=2.4 Hz, 1H), 8.27 (d, J=8.4 Hz, 1H), 8.13 (dd, J=8.4, 2.4 Hz, 1H), 8.04-7.97 (m, 4H), 7.50-7.44 (m, 6H), 7.43-7.35 (m, 1H), 7.15 (dd, J=8.0, 2.0 Hz, 1H), 7.02 (br s, 2H), 6.98 (d, J=8.8 Hz, 1H), 6.38 (dd, J=7.6, 4.8 Hz, 1H), 5.15-5.09 (m, 1H), 3.61 (s, 2H), 2.77-2.72 (m, 2H), 2.34-2.28 (m, 2H), 2.05-1.99 (m, 2H), 1.77-1.71 (m, 2H).

### Example 3: 6-((1-(4-(2-(2-aminopyridin-3-yl)-6-phenyl-3H-imidazo[4,5-b]pyridin-3-yl)benzyl)piperidin-4-yl)oxy)nicotinonitrile

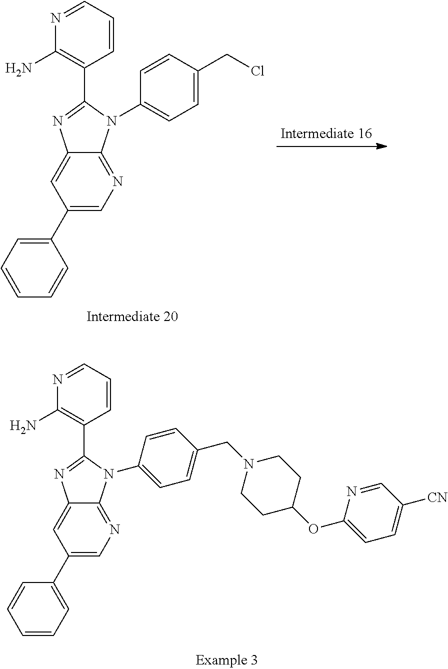 [SMILES: Nc1ncccc1-c1nc2cc(-c3ccccc3)cnc2n1-c1ccc(CCl)cc1 ; N#Cc1ccc(OC2CCN(Cc3ccc(-n4c(-c5cccnc5N)nc5cc(-c6ccccc6)cnc54)cc3)CC2)nc1]

Following the general procedure of Example 2, the reaction of Intermediate 20 (200 mg, 486 μmol) with Intermediate 16 (109 mg, 534 μmol) was carried out. After purified by prep-HPLC (column: Waters Xbridge 150*25 mm*5 μm; mobile phase: [water (NH₄HCO₃)-ACN]; B %: 54%-84%, 8 min), 6-((1-(4-(2-(2-aminopyridin-3-yl)-6-phenyl-3H-imidazo[4,5-b]pyridin-3-yl)benzyl)piperidin-4-yl)oxy)nicotinonitrile (Example 3, 10.2 mg, yield: 3.6%) was obtained as a yellow oil. MS: m/z=579.1 [M+H]⁺. ¹H NMR (400 MHz, Chloroform-d) δ 8.67 (s, 1H), 8.50-8.46 (m, 1H), 8.34 (s, 1H), 8.08-7.98 (m, 2H), 7.91 (d, J=3.6 Hz, 1H), 7.84 (dd, J=8.0, 2.0 Hz, 1H), 7.64 (d, J=7.2 Hz, 2H), 7.57-7.49 (m, 6H), 7.46-7.36 (m, 2H), 6.85 (d, J=8.8 Hz, 1H), 6.67-6.41 (m, 1H), 5.64-5.34 (m, 1H), 4.26 (s, 2H), 3.58-3.30 (m, 2H), 3.27-2.98 (m, 2H), 2.90-2.66 (m, 2H), 2.36-2.24 (m, 2H).

### Example 4: 6-((1-(4-(2-(2-Aminopyridin-3-yl)-3H-imidazo[4,5-b]pyridin-3-yl)benzyl)piperidin-4-yl)oxy)picolinonitrile

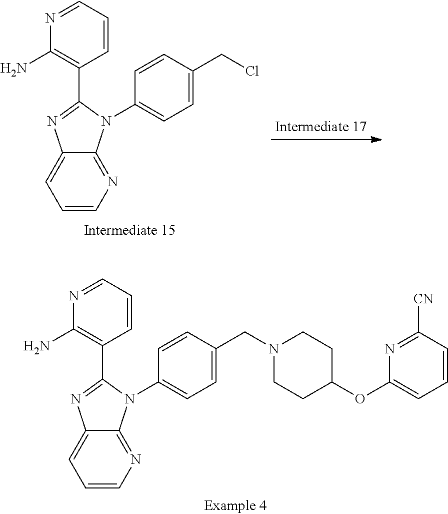 [SMILES: Nc1ncccc1-c1nc2cccnc2n1-c1ccc(CCl)cc1 ; N#Cc1cccc(OC2CCN(Cc3ccc(-n4c(-c5cccnc5N)nc5cccnc54)cc3)CC2)n1]

To a solution of Intermediate 15 (150 mg, 0.447 mmol) and Intermediate 17 (100 mg, 0.491 mmol) in DMF (3 mL) was added NaI (7 mg, 45 μmol) and K₂CO₃ (123 mg, 0.893 mmol). The mixture was stirred at 80° C. for 2 hr. The mixture was diluted with H₂O (10 mL) and extracted with EtOAc (10 mL×2). The combined organic layers were washed with brine (10 mL), dried over Na₂SO₄, filtered, and concentrated under reduced pressure to give a residue. The residue was purified by prep-HPLC (column: Welch Ultimate C18 150*25 mm*5 μm; mobile phase: [water (NH₄HCO₃)—CAN]; B %: 34%-64%, 10 min), 6-((1-(4-(2-(2-Aminopyridin-3-yl)-3H-imidazo[4,5-b]pyridin-3-yl)benzyl)piperidin-4-yl)oxy)picolinonitrile (Example 4, 32.8 mg, yield: 14.6%) was obtained as a light-yellow solid. MS: m/z=503.3 [M+H]⁺. ¹H NMR (400 MHz, Dimethylsulfoxide-d₆) δ 8.33 (dd, J=4.8, 1.2 Hz, 1H), 8.20 (dd, J=8.0, 1.2 Hz, 1H), 7.99 (dd, 1=4.4, 1.6 Hz, 1H), 7.91 (dd, J=8.4, 7.2 Hz, 1H), 7.62 (d, J=7.2 Hz, 1H), 7.46 d, J=8.4 Hz, 2H), 7.41-7.37 (m, 3H), 7.18-7.15 (m, 2H), 7.01 (br s, 2H), 6.38 (dd, J=7.6, 4.8 Hz, 1H), 5.08-5.00 (m, 1H), 3.59 (s, 2H), 2.76-2.69 (m, 2H), 2.36-2.28 (m, 2H), 2.05-1.97 (m, 2H), 1.77-1.68 (m, 2H).

### Example 5: 2-((1-(4-(2-(2-aminopyridin-3-yl)-3H-imidazo[4,5-b]pyridin-3-yl)benzyl)piperidin-4-yl)oxy)isonicotinonitrile

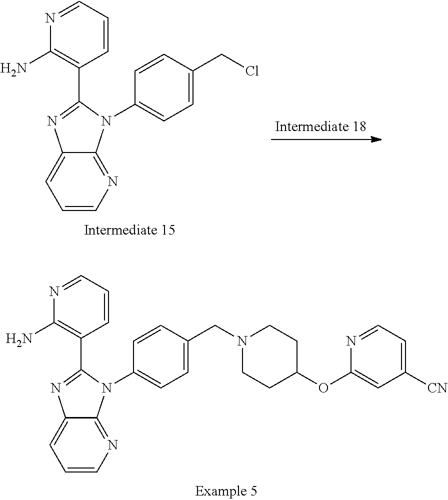 [SMILES: Nc1ncccc1-c1nc2cccnc2n1-c1ccc(CCl)cc1 ; N#Cc1ccnc(OC2CCN(Cc3ccc(-n4c(-c5cccnc5N)nc5cccnc54)cc3)CC2)c1]

Following the general procedure of Example 4, the reaction of Intermediate 15 (150 mg, 0.447 mmol) with Intermediate 18 (100 mg, 0.491 mmol) was carried out. After purified by prep-HPLC (column: Welch Xtimate C18 150*25 mm*5 μm; mobile phase: [water (HCl)-CAN]; B %: 0%-27%, 11 min), 2-((1-(4-(2-(2-aminopyridin-3-yl)-3H-imidazo[4,5-b]pyridin-3-yl)benzyl)piperidin-4-yl)oxy)isonicotinonitrile (Example 5, 12 mg, yield: 5.3%) was obtained as a white solid. MS: m/z=503.3 [M+H]⁺. ¹H NMR (400 MHz, Dimethylsulfoxide-d₆) δ 8.39 (d, J=5.2 Hz, 1H), 8.33 (d, J=4.4 Hz, 1H), 8.20 (d, J=8.0 Hz, 1H), 7.98 (d, J=3.6 Hz, 1H), 7.45 (d, J=8.4 Hz, 2H), 7.40-7.34 (m, 5H), 7.16 (d, J=8.0 Hz, 1H), 7.00 (br s, 2H), 6.37 (dd, J=7.6, 4.8 Hz, 1H), 5.09-5.03 (m, 1H), 3.58 (s, 2H), 2.77-2.69 (m, 2H), 2.32-2.26 (m, 2H), 2.04-1.98 (m, 2H), 1.75-1.68 (m, 2H).

### Example 6: 2-((1-(4-(2-(2-aminopyridin-3-yl)-3H-imidazo[4,5-b]pyridin-3-yl)benzyl)piperidin-4-yl)oxy)nicotinonitrile

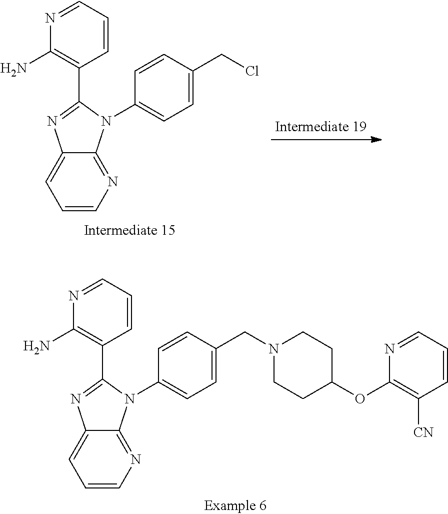 [SMILES: Nc1ncccc1-c1nc2cccnc2n1-c1ccc(CCl)cc1 ; N#Cc1cccnc1OC1CCN(Cc2ccc(-n3c(-c4cccnc4N)nc4cccnc43)cc2)CC1]

Following the general procedure of Example 4, the reaction of Intermediate 15 (150 mg, 0.447 mmol) with Intermediate 19 (100 mg, 0.491 mmol) was carried out. After purified by prep-HPLC (column: Welch Xtimate C18 150×25 mm 5 μm; mobile phase: [water (HCl)-ACN]; B %: 0%-27%, 11 min), 2-((1-(4-(2-(2-aminopyridin-3-yl)-3H-imidazo[4,5-b]pyridin-3-yl)benzyl)piperidin-4-yl)oxy)nicotinonitrile (Example 6, 24.5 mg, yield: 11%) was obtained as a white solid. MS: m/z=503.4 [M+H]^{+ 1}H NMR (400 MHz, Dimethylsulfoxide-d₆) δ 8.45 (d, J=4.8 Hz, 1H), 8.33 (d, J=4.8 Hz, 1H), 8.26 (d, J=7.6 Hz, 1H), 8.20 (d, J=8.0 Hz, 1H), 7.98 (d, J=4.0 Hz, 1H), 7.48 (d, J=8.4 Hz, 2H), 7.41-7.36 (m, 3H), 7.18-7.14 (m, 2H), 7.00 (br s, 2H), 6.38 (dd, J=8.0, 4.8 Hz, 1H), 5.25-5.16 (m, 1H), 3.59 (s, 2H), 2.73-2.66 (m, 2H), 2.40-2.32 (m, 2H), 2.05-1.98 (m, 2H), 1.82-1.74 (m, 2H).

### Example 7: 6-((1-(4-(2-(2-Aminopyridin-3-yl)-5-phenyl-3H-imidazo[4,5-b]pyridin-3-yl)benzyl)piperidin-4-yl)oxy)picolinonitrile

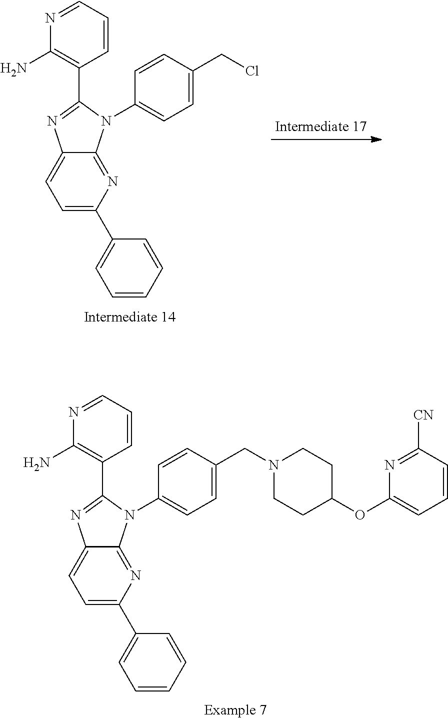 [SMILES: Nc1ncccc1-c1nc2ccc(-c3ccccc3)nc2n1-c1ccc(CCl)cc1 ; N#Cc1cccc(OC2CCN(Cc3ccc(-n4c(-c5cccnc5N)nc5ccc(-c6ccccc6)nc54)cc3)CC2)n1]

To a solution of Intermediate 14 (150 mg, 364 μmol) and Intermediate 17 (81.4 mg, 401 μmol) in DMF (1 mL) was added NaI (5.46 mg, 36.4 μmol) and K₂CO₃ (101 mg, 728 μmol). The mixture was stirred at 80° C. for 2 hr. Then the mixture was purified by prep-HPLC (column: Phenomenex C18 150*25 mm*10 μm; mobile phase: [water (NH₄HCO₃)-ACN]; B %: 60%-90%, 8 min), 6-((1-(4-(2-(2-aminopyridin-3-yl)-5-phenyl-3H-imidazo[4,5-b]pyridin-3-yl)benzyl)piperidin-4-yl)oxy)picolinonitrile (Example 7, 11.5 mg, yield: 5.5%) as light-yellow solid. MS: m/z=579.3 [M+H]⁺. ¹H NMR (400 MHz, Dimethylsulfoxide-d₆) δ 8.27 (d, J=8.4 Hz, 1H), 8.06-7.96 (m, 4H), 7.95-7.88 (m, 1H), 7.62 (d, J=7.6 Hz, 1H), 7.52-7.43 (m, 6H), 7.41-7.39 (m, 1H), 7.19-7.12 (m, 2H), 7.02 (br s, 2H), 6.38 (dd, J=8.0, 5.2 Hz, 1H), 5.10-4.96 (m, 1H), 3.62 (s, 2H), 2.75-2.71 (m, 2H), 2.23-2.31 (m, 2H), 2.04-1.99 (m, 2H), 1.76-1.68 (m, 2H).

### Example 8: 2-((1-(4-(2-(2-Aminopyridin-3-yl)-5-phenyl-3H-imidazo[4,5-b]pyridin-3-yl)benzyl)piperidin-4-yl)oxy)isonicotinonitrile

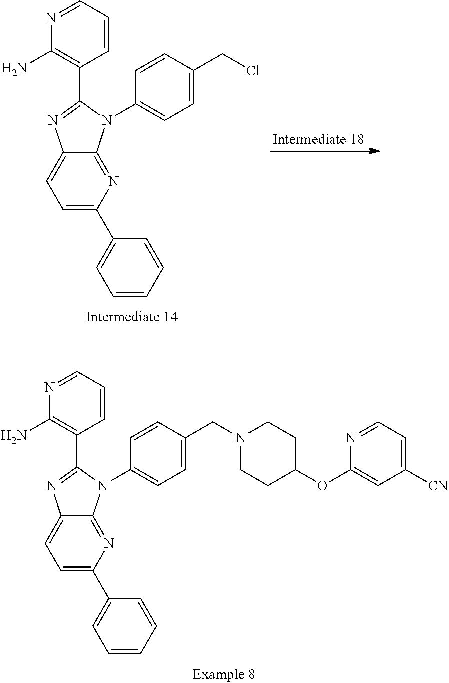 [SMILES: Nc1ncccc1-c1nc2ccc(-c3ccccc3)nc2n1-c1ccc(CCl)cc1 ; N#Cc1ccnc(OC2CCN(Cc3ccc(-n4c(-c5cccnc5N)nc5ccc(-c6ccccc6)nc54)cc3)CC2)c1]

Following the general procedure of Example 7, Intermediate 14 (100 mg, 243 μmol) with Intermediate 18 (54.3 mg, 267 μmol) was carried out. After purified by prep-HPLC (column: Phenomenex C18 150*25 mm*10 μm; mobile phase: [water (NH₄HCO₃)-ACN]; B %: 58%-88%, 8 min), 2-((1-(4-(2-(2-aminopyridin-3-yl)-5-phenyl-3H-imidazo[4,5-b]pyridin-3-yl)benzyl)piperidin-4-yl)oxy)isonicotinonitrile (Example 8, 18.1 mg, yield: 13%) was obtained as an off-white solid. MS: m/z=579.1 [M+H]⁺. ¹H NMR (400 MHz, Dimethylsulfoxide-d₆) δ 8.39 (d, J=5.2 Hz, 1H), 8.27 (d, J=8.4 Hz, 1H), 8.06-7.97 (m, 4H), 7.51-7.44 (m, 6H), 7.41-7.34 (m, 3H), 7.16 (d, J=7.6 Hz, 1H), 7.02 (br s, 2H), 6.38 (dd, J=8.0, 4.8 Hz, 1H), 5.10-5.03 (m, 1H), 3.61 (s, 2H), 2.77-2.73 (m, 2H), 2.34-2.24 (m, 2H), 2.05-1.99 (m, 2H), 1.77-1.68 (m, 2H).

### Example 9: 2-((1-(4-(2-(2-Aminopyridin-3-yl)-5-phenyl-3H-imidazo[4,5-b]pyridin-3-yl)benzyl)piperidin-4-yl)oxy)nicotinonitrile

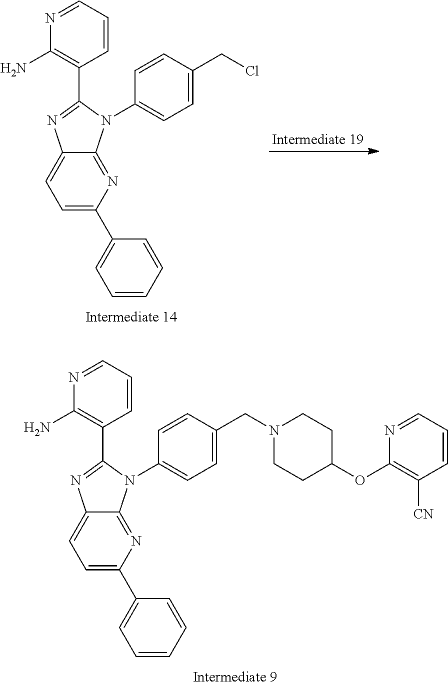 [SMILES: Nc1ncccc1-c1nc2ccc(-c3ccccc3)nc2n1-c1ccc(CCl)cc1 ; N#Cc1cccnc1OC1CCN(Cc2ccc(-n3c(-c4cccnc4N)nc4ccc(-c5ccccc5)nc43)cc2)CC1]

Following the general procedure of Example 7, the reaction of Intermediate 14 (100 mg, 243 μmol) with Intermediate 19 (54.3 mg, 267 μmol) was carried out. After purified by prep-HPLC (column: Phenomenex C18 150*25 mm*10 μm; mobile phase: [water (NH₄HCO₃)-ACN]; B %: 58%-88%, 8 min), 2-((1-(4-(2-(2-aminopyridin-3-yl)-5-phenyl-3H-imidazo[4,5-b]pyridin-3-yl)benzyl)piperidin-4-yl)oxy)nicotinonitrile (Example 9, 15 mg, yield: 11%) was obtained as an off-white solid. MS: m/z=579.1 [M+H]⁺. ¹H NMR (400 MHz, Dimethylsulfoxide-d₆) δ 8.46 (dd, J=5.2, 2.0 Hz, 1H), 8.30-8.23 (m, 2H), 8.05-7.97 (m, 4H), 7.53-7.44 (m, 6H), 0.7.41-7.39 (m, 1H), 7.19-7.13 (m, 2H), 7.02 (br s, 2H), 6.38 (dd, J=8.0, 5.2 Hz, 1H), 5.28-5.16 (m, 1H), 3.62 (s, 2H), 2.68-2.66 (m, 2H), 2.40-2.35 (m, 2H), 2.06-2.00 (m, 2H), 1.83-1.75 (m, 2H).

### Example 10: 6-((1-(4-(2-(2-Aminopyridin-3-yl)-6-phenyl-3H-imidazo[4,5-b]pyridin-3-yl)benzyl)piperidin-4-yl)oxy)picolinonitrile

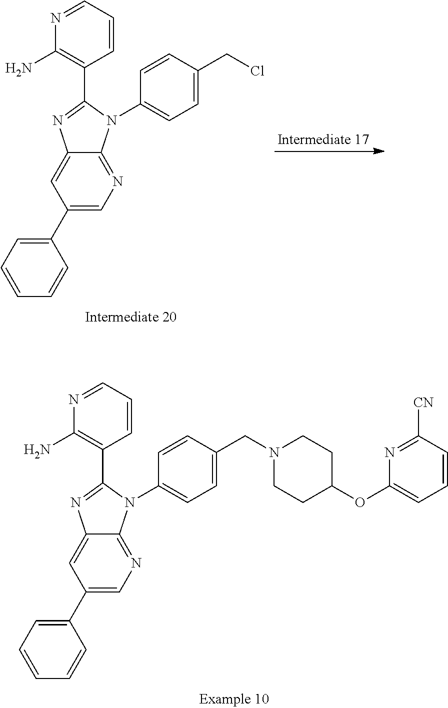 [SMILES: Nc1ncccc1-c1nc2cc(-c3ccccc3)cnc2n1-c1ccc(CCl)cc1 ; N#Cc1cccc(OC2CCN(Cc3ccc(-n4c(-c5cccnc5N)nc5cc(-c6ccccc6)cnc54)cc3)CC2)n1]

Following the general procedure of Example 7, the reaction of Intermediate 20 (100 mg, 243 μmol) with Intermediate 17 (54 mg, 267 μmol) was carried out. After purified by prep-HPLC (column: Phenomenex C18 150*25 mm*10 μm; mobile phase: [water (NH₄HCO₃)-ACN]; B %: 56%-86%, 8 min), 6-((1-(4-(2-(2-aminopyridin-3-yl)-6-phenyl-3H-imidazo[4,5-b]pyridin-3-yl)benzyl)piperidin-4-yl)oxy)picolinonitrile (Example 10, 11 mg, yield: 7.8%) was obtained as a yellow solid. MS: m/z=579.3 [M+H]⁺. ¹H NMR (400 MHz, Dimethylsulfoxide-d₆) δ 8.64 (d, J=2.0 Hz, 1H), 8.47 (d, J=2.0 Hz, 1H), 8.00 (dd, J=4.8, 1.6 Hz, 1H), 7.92 (dd, J=8.0, 8.0 Hz, 1H), 7.78 (d, J=7.6 Hz, 2H), 7.62 (d, J=7.2 Hz, 1H), 7.54-7.47 (m, 4H), 7.45-7.40 (m, 3H), 7.25-7.13 (m, 2H), 7.05 (br s, 2H), 6.39 (dd, J=7.6, 4.8 Hz, 1H), 5.09-5.01 (m, 1H), 3.60 (s, 2H), 2.76-2.70 (m, 2H), 2.37-2.29 (m, 2H), 2.07-1.99 (m, 2H), 1.77-1.68 (m, 2H).

### Example 11: 2-((1-(4-(2-(2-Aminopyridin-3-yl)-6-phenyl-3H-imidazo[4,5-h]pyridin-3-yl)benzyl)piperidin-4-yl)oxy)isonicotinonitrile

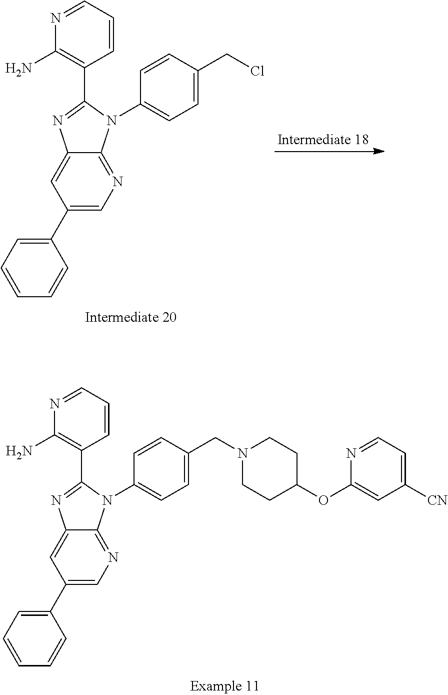 [SMILES: Nc1ncccc1-c1nc2cc(-c3ccccc3)cnc2n1-c1ccc(CCl)cc1 ; N#Cc1ccnc(OC2CCN(Cc3ccc(-n4c(-c5cccnc5N)nc5cc(-c6ccccc6)cnc54)cc3)CC2)c1]

Following the general procedure of Example 7, the reaction of Intermediate 20 (100 mg, 243 μmol) with Intermediate 18 (54 mg, 267 μmol) was carried out. After purified by prep-HPLC (column: Phenomenex C18 150*25 mm*10 μm; mobile phase: [water (NH₄HCO₃)-ACN]; B %: 50%-80%, 8 min), 2-((1-(4-(2-(2-aminopyridin-3-yl)-6-phenyl-3H-imidazo[4,5-b]pyridin-3-yl)benzyl)piperidin-4-yl)oxy)isonicotinonitrile (Example 11, 10.6 mg, yield: 7.5%) was obtained as a yellow solid. MS: m/z=579.3 [M+H]⁺. ¹H NMR (400 MHz, Dimethylsulfoxide-d₆) δ 8.63 (d, J=2.0 Hz, 1H), 8.46 (d, J=2.0 Hz, 1H), 8.39 (d, J=5.2 Hz, 1H), 8.03-7.94 (m, 1H), 7.78 (d, J=7.2 Hz, 2H), 7.55-7.47 (m, 4H), 7.45-7.39 (m, 3H), 7.39-7.32 (m, 2H), 7.20 (dd, J=7.6, 1.2 Hz, 1H), 7.04 (br s, 2H), 6.39 (dd, J=7.6, 4.8 Hz, 1H), 5.11-5.03 (m, 1H), 3.61 (s, 2H), 2.84-2.72 (m, 2H), 2.39-2.19 (m, 2H), 2.03-1.95 (m, 2H), 1.76-1.65 (m, 2H).

### Example 12: 2-((1-(4-(2-(2-Aminopyridin-3-yl)-6-phenyl-3H-imidazo[4,5-b]pyridin-3-yl)benzyl)piperidin-4-yl)oxy)nicotinonitrile

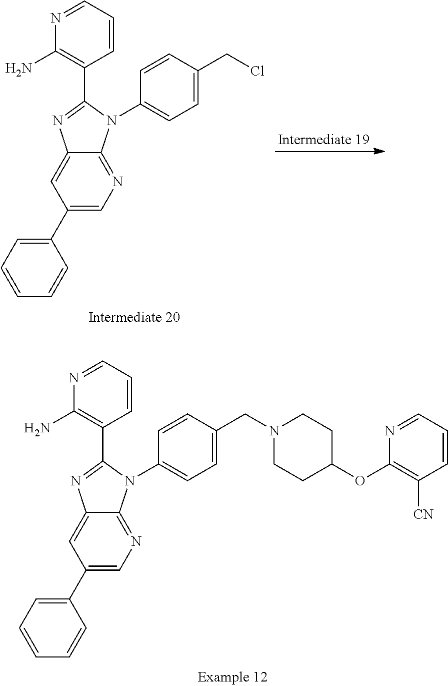 [SMILES: Nc1ncccc1-c1nc2cc(-c3ccccc3)cnc2n1-c1ccc(CCl)cc1 ; N#Cc1cccnc1OC1CCN(Cc2ccc(-n3c(-c4cccnc4N)nc4cc(-c5ccccc5)cnc43)cc2)CC1]

Following the general procedure of Example 7, the reaction of Intermediate 20 (170 mg, 413 μmol) with Intermediate 19 (93 mg, 454 μmol) was carried out. After purified by prep-HPLC (column: Phenomenex C18 150*25 mm*10 μm; mobile phase: [water (NH₄HCO₃)-ACN]; B %: 52%-82%, 8 min), 2-((1-(4-(2-(2-aminopyridin-3-yl)-6-phenyl-3H-imidazo[4,5-b]pyridin-3-yl)benzyl)piperidin-4-yl)oxy)nicotinonitrile (Example 12, 15.6 mg, yield: 6.5%) was obtained as a yellow solid. MS: m/z=579.2 [M+H]⁺. ¹H NMR (400 MHz, Dimethylsulfoxide-d₆) δ 8.64 (d, J=1.8 Hz, 1H), 8.49-8.43 (m, 2H), 8.26 (dd, J=7.6, 1.6 Hz, 1H), 8.00 (dd, J=4.8, 1.6 Hz, 1H), 7.79 (d, J=7.6 Hz, 2H), 7.54-7.48 (m, 4H), 7.44-7.40 (m, 3H), 7.21-7.14 (m, 2H), 7.05 (br s, 2H), 6.39 (dd, J=7.6, 4.8 Hz, 1H), 5.26-5.16 (m, 1H), 3.60 (s, 2H), 2.75-2.63 (m, 2H), 2.39-2.30 (m, 2H), 2.09-1.97 (m, 2H), 1.83-1.74 (m, 2H).

### Example 13: 4-(4-(4-(2-(2-Aminopyridin-3-yl)-3H-imidazo[4,5-b]pyridin-3-yl)benzyl)piperazin-1-yl)picolinonitrile

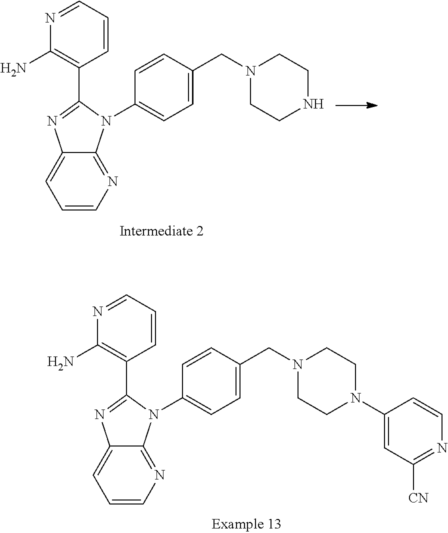 [SMILES: Nc1ncccc1-c1nc2cccnc2n1-c1ccc(CN2CCNCC2)cc1 ; N#Cc1cc(N2CCN(Cc3ccc(-n4c(-c5cccnc5N)nc5cccnc54)cc3)CC2)ccn1]

To a solution of Intermediate 2 (200 mg, 519 μmol) in DMF (2 mL) was added DIEA (201 mg, 1.6 mmol), and 4-fluoropyridine-2-carbonitrile (76 mg, 623 μmol). The mixture was stirred at 100° C. for 2 hr. The mixture was diluted with H₂O (10 mL) and extracted with EtOAc (5 mL×2). The combined organic layers were washed with brine (10 mL), dried over Na₂SO₄, filtered, and concentrated under reduced pressure to give a residue. The crude product was purified by prep-HPLC (column: Phenomenex luna C18 150*25 mm*10 μm; mobile phase: [water (FA)-ACN]; B %: 0%-23%, 9 min), 4-(4-(4-(2-(2-aminopyridin-3-yl)-3H-imidazo[4,5-b]pyridin-3-yl)benzyl)piperazin-1-yl)picolinonitrile (Example 13, 9.6 mg, yield: 3.8%) was obtained as a yellow solid. MS: m/z=488.1 [M+H]⁺. ¹H NMR (400 MHz, Chloroform-d) δ 8.44 (dd, J=4.8, 4.2 Hz, 1H), 8.30 (d, J=6.0 Hz, 1H), 8.14 (dd, J=8.0, 1.2 Hz, 1H), 7.97-7.93 (m, 1H), 7.82 (br s, 2H), 7.56 (d, J=8.4 Hz, 2H), 7.42-7.32 (m, 3H), 7.21 (dd, J=7.6, 1.6 Hz, 1H), 7.04 (d, J=2.8 Hz, 1H), 6.80-6.74 (m, 1H), 6.40 (dd, J=7.6, 4.8 Hz, 1H), 3.67 (s, 2H), 3.44-3.39 (m, 4H), 2.68-2.62 (m, 4H).

### Example 14: 5-(4-(4-(2-(2-Aminopyridin-3-yl)-3H-imidazo[4,5-b]pyridin-3-yl)benzyl)piperazin-1-yl)picolinonitrile

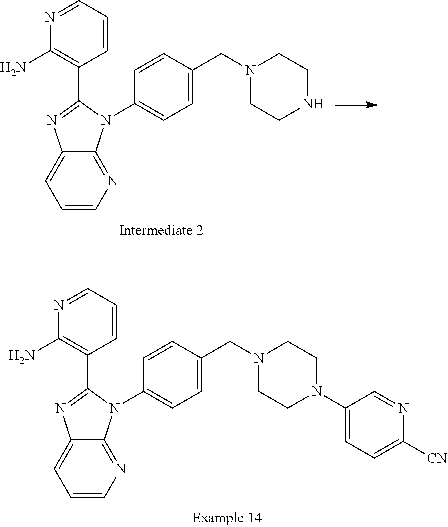 [SMILES: Nc1ncccc1-c1nc2cccnc2n1-c1ccc(CN2CCNCC2)cc1 ; N#Cc1ccc(N2CCN(Cc3ccc(-n4c(-c5cccnc5N)nc5cccnc54)cc3)CC2)cn1]

To a solution of Intermediate 2 (200 mg, 519 μmol) and 5-fluoropyridine-2-carbonitrile (76 mg, 623 μmol) in DMF (2 mL) was added DIEA (325 μmol). The reaction mixture stirred at 100° C. for 16 hr. Then the mixture was purified by prep-HPLC (column: Waters xbridge 150*25 mm 10 um; mobile phase: [water (NH₄HCO₃)-ACN]; B %: 24%-54%, 8 min), 5-(4-(4-(2-(2-aminopyridin-3-yl)-3H-imidazo[4,5-b]pyridin-3-yl)benzyl)piperazin-1-yl)picolinonitrile (Example 14, 21.8 mg, yield: 8.3%) as a yellow solid. MS: m/z=488.2 [M+H]⁺. ¹H NMR (400 MHz, Dimethylsulfoxide-d₆) δ 8.43 (d, J=2.8 Hz, 1H), 8.34 (dd, J=4.8, 1.2 Hz, 1H), 8.21 (dd, J=8.0, 1.2 Hz, 1H), 7.99 (dd, J=4.8, 1.2 Hz, 1H), 7.75 (d, J=8.8 Hz, 1H), 7.49 (d, J=8.4 Hz, 2H), 7.42-7.34 (m, 4H), 7.17 (dd, J=7.6, 1.6 Hz, 1H), 7.01 (br s, 2H), 6.37 (dd, J=7.6, 4.8 Hz, 1H), 3.63 (s, 2H), 3.50-3.40 (m, 4H), 2.55-2.48 (m, 4H).

### Example 15: 6-(4-(4-(2-(2-Aminopyridin-3-yl)-3H-imidazo[4,5-b]pyridin-3-yl)benzyl)piperazin-1-yl)picolinonitrile

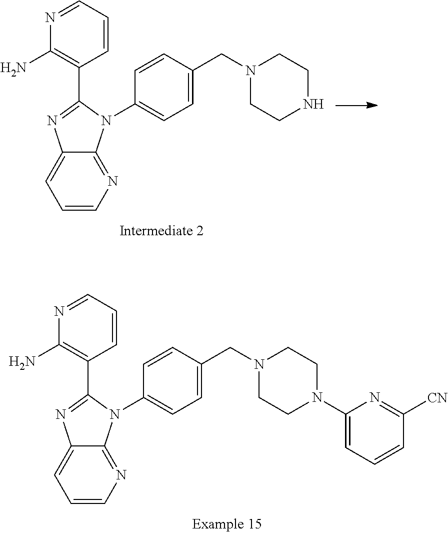 [SMILES: Nc1ncccc1-c1nc2cccnc2n1-c1ccc(CN2CCNCC2)cc1 ; N#Cc1cccc(N2CCN(Cc3ccc(-n4c(-c5cccnc5N)nc5cccnc54)cc3)CC2)n1]

A mixture of Intermediate 2 (200 mg, 519 μmol), 6-fluoropicolinonitrile (76 mg, 622 μmol) and DIEA (134 mg, 1.0 mmol) in DMSO (3 mL) was stirred at 120° C. for 24 hr. The reaction mixture was poured into H₂O (50 mL). The resulting mixture was extracted with CH₂Cl₂ (50 mL×3). The combined organic layers were dried over anhydrous Na₂SO₄, filtered, and concentrated to dryness. The residue was purified by prep-HPLC (column: Waters Xbridge BEH C18 250*50 mm*10 μm; mobile phase: [water (ammonia hydroxide v/v)-ACN]; B %: 31%-61%, 0 min), 6-(4-(4-(2-(2-aminopyridin-3-yl)-3H-imidazo[4,5-b]pyridin-3-yl)benzyl)piperazin-1-yl)picolinonitrile (Example 15, 10.5 mg, yield: 4.2%) as a light-yellow lyophilized powder. MS: m/z=488.3 [M+H]⁺. ¹H NMR (400 MHz, Chloroform-d) δ 8.41 (d, J=4.4 Hz, 1H), 8.02-8.16 (m, 2H), 7.57-7.48 (m, 3H), 7.42-7.30 (m, 3H), 7.09 (d, J=7.6 Hz, 1H), 6.97 (d, J=7.2 Hz, 1H), 6.82 (d, J=8.8 Hz, 1H), 6.63 (br s, 2H), 6.40-6.31 (m, 1H), 3.70-3.54 (m, 6H), 2.65-2.53 (m, 2H), 2.06-1.96 (m, 2H).

### Example 16: 5-(4-(4-(2-(2-Aminopyridin-3-yl)-3H-imidazo[4,5-b]pyridin-3-yl)benzyl)piperazin-1-yl)nicotinonitrile

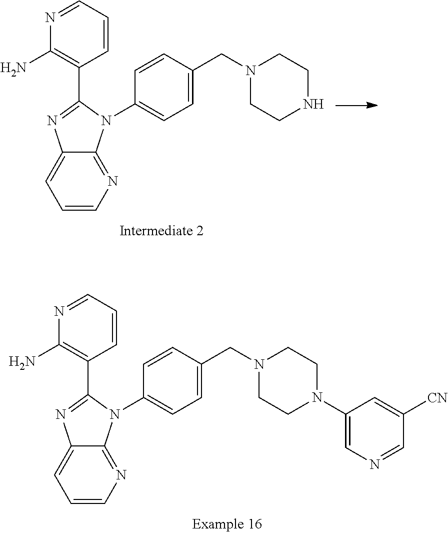 [SMILES: Nc1ncccc1-c1nc2cccnc2n1-c1ccc(CN2CCNCC2)cc1 ; N#Cc1cncc(N2CCN(Cc3ccc(-n4c(-c5cccnc5N)nc5cccnc54)cc3)CC2)c1]

Following the general procedure of Example 15, the reaction of Intermediate 2 (200 mg, 519 μmol) with 5-fluoronicotinonitrile (63.3 mg, 519 μmol) was carried out. After purified by prep-HPLC (column: Waters xbridge 150*25 mm 10 μm; mobile phase: [water (NH₄HCO₃)-ACN]; B %: 23%-53%, 11 min), 5-(4-(4-(2-(2-aminopyridin-3-yl)-3H-imidazo[4,5-b]pyridin-3-yl)benzyl)piperazin-1-yl)nicotinonitrile (Example 16, 5.2 mg, yield: 2.0%) was obtained as a yellow solid. MS: m/z=488.3 [M+H]⁺. ¹H NMR (400 MHz, Dimethylsulfoxide-d₆) δ 8.59 (d, J=2.8 Hz, 1H), 8.40-8.28 (m, 2H), 8.21 (dd, J=8.0, 1.2 Hz, 1H), 7.99 (dd, J=4.8, 1.6 Hz, 1H), 7.77 (s, 1H), 7.49 (d, J=8.4 Hz, 2H), 7.44-7.36 (m, 3H), 7.17 (dd, J=8.0, 2.0 Hz, 1H), 7.01 (br s, 2H), 6.38 (dd, J=7.6, 4.8 Hz, 1H), 3.62 (s, 2H), 3.37-3.33 (m, 4H), 2.58-2.52 (m, 4H).

### Example 17: 2-(4-(4-(2-(2-Aminopyridin-3-yl)-3H-imidazo[4,5-b]pyridin-3-yl)benzyl)piperazin-1-yl)isonicotinonitrile

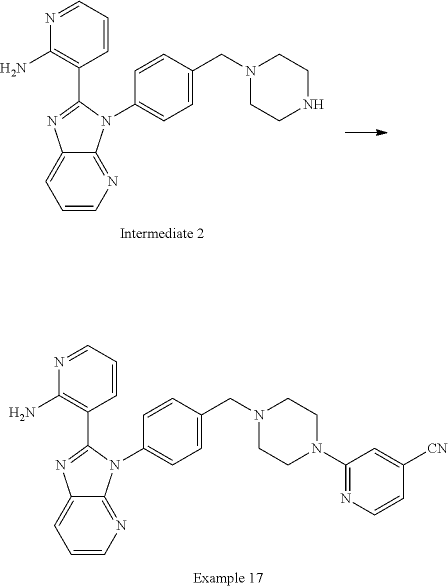 [SMILES: Nc1ncccc1-c1nc2cccnc2n1-c1ccc(CN2CCNCC2)cc1 ; N#Cc1ccnc(N2CCN(Cc3ccc(-n4c(-c5cccnc5N)nc5cccnc54)cc3)CC2)c1]

A solution of Intermediate 2 (200 mg, 519 μmol), 2-chloroisonicotinonitrile (143 mg, 1.03 mmol) and DIEA (134 mg, 1.0 mmol) in NMP (3 mL) were taken up into a microwave tube at 25° C. The sealed tube was heated at 160° C. for 1 hr under microwave. The reaction mixture was poured into H₂O (50 mL). The resulting mixture was extracted with CH₂Cl₂ (50 mL×3). The combined organic dried over anhydrous Na₂SO₄, filtered and concentrated to dryness. The residue was purified by prep-HPLC (column: Waters xbridge 150*25 mm 10 μm; mobile phase: [water (NH₄HCO₃)-ACN]; B %: 30%-60%, 8 min), 2-(4-(4-(2-(2-aminopyridin-3-yl)-3H-imidazo[4,5-b]pyridin-3-yl)benzyl)piperazin-1-yl)isonicotinonitrile (Example 17, 13.1 mg, yield: 5.2%) was obtained as a yellow lyophilized powder. MS: m/z=488.3 [M+H]⁺. ¹H NMR (400 MHz, Dimethylsulfoxide-d₆) δ 8.34 (dd, J=4.8, 1.2 Hz, 1H), 8.29 (d, J=5.2 Hz, 1H), 8.21 (dd, J=8.0, 1.2 Hz, 1H), 7.99 (dd, =4.8, 1.6 Hz, 1H), 7.49 (d, J=8.0 Hz, 2H), 7.42-7.37 (m, 3H), 7.31 (s, 1H), 7.16 (dd, J=8.0, 2.0 Hz, 1H), 7.01 (br s, 2H), 6.95 (d, J=5.2 Hz, 1H), 6.36 (dd, J=7.6, 4.8 Hz, 1H), 3.63-3.57 (m, 6H), 2.49-2.43 (m, 4H).

### Example 18: 4-(4-(4-(2-(2-Aminopyridin-3-yl)-3H-imidazo[4,5-b]pyridin-3-yl)benzyl)piperazin-1-yl)pyrimidine-2-carbonitrile

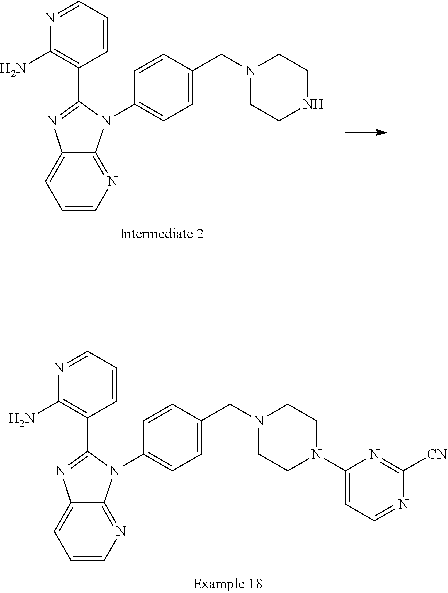 [SMILES: Nc1ncccc1-c1nc2cccnc2n1-c1ccc(CN2CCNCC2)cc1 ; N#Cc1nccc(N2CCN(Cc3ccc(-n4c(-c5cccnc5N)nc5cccnc54)cc3)CC2)n1]

Following the general procedure of Example 17, the reaction of Intermediate 2 (200 mg, 519 μmol) with 4-chloropyrimidine-2-carbonitrile (72.4 mg, 519 μmol) was carried out at 130° C. After purified by prep-HPLC (column: Waters xbridge 150*25 mm 10 μm; mobile phase: [water (NH₄HCO₃)-ACN]; B %: 28%-58%, 10 min), 4-(4-(4-(2-(2-aminopyridin-3-yl)-3H-imidazo[4,5-b]pyridin-3-yl)benzyl)piperazin-1-yl)pyrimidine-2-carbonitrile (Example 18, 10.5 mg, yield: 4.1%) was obtained as a brown lyophilized powder. MS: m/z=489.4 [M+H]⁺. ¹H NMR (400 MHz, Dimethylsulfoxide-d₆) δ 8.33 (dd, J=4.8, 1.6 Hz, 1H), 8.26 (d, J=6.4 Hz, 1H), 8.20 (dd, J=8.0, 1.2 Hz, 1H), 7.98 (dd, J=4.8, 2.0 Hz, 1H), 7.48 (d, J=8.0 Hz, 2H), 7.42-7.36 (m, 3H), 7.16 (dd, J=7.6, 1.6 Hz, 1H), 7.10 (d, J=6.4 Hz, 1H), 7.00 (br s, 2H), 6.36 (dd, J=7.6, 4.8 Hz, 1H), 3.76-3.63 (m, 4H), 3.62 (s, 2H), 2.49-2.43 (m, 4H).

### Example 19: 6-(4-(4-(2-(2-Aminopyridin-3-yl)-3H-imidazo[4,5-b]pyridin-3-yl)benzyl)piperazin-1-yl)pyrazine-2-carbonitrile

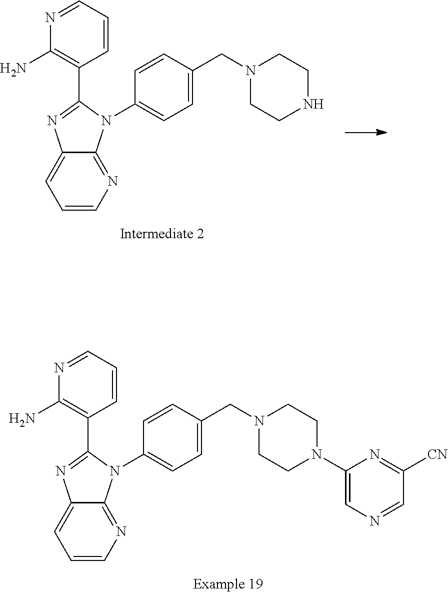 [SMILES: Nc1ncccc1-c1nc2cccnc2n1-c1ccc(CN2CCNCC2)cc1 ; N#Cc1cncc(N2CCN(Cc3ccc(-n4c(-c5cccnc5N)nc5cccnc54)cc3)CC2)n1]

Following the general procedure of Example 17, the reaction of Intermediate 2 (200 mg, 519 μmol) with 6-chloropyrazine-2-carbonitrile (144 mg, 1.03 mmol) was carried out. After purified by prep-HPLC (column. Waters xbridge 150*25 mm 10 μm; mobile phase: [water (NH₄HCO₃)-ACN]; B %: 27%-57%, 8 min), 6-(4-(4-(2-(2-aminopyridin-3-yl)-3H-imidazo[4,5-b]pyridin-3-yl)benzyl)piperazin-1-yl)pyrazine-2-carbonitrile (Example 19, 50.5 mg, yield: 20%) was obtained as a yellow solid. MS: m/z=489.3 [M+H]⁺. ¹H NMR (400 MHz, Dimethylsulfoxide-d₆) δ 8.65 (s, 1H), 8.34 (dd, J=4.4, 1.2 Hz, 1H), 8.30 (s, 1H), 8.21 (dd, J=8.0, 1.6 Hz, 1H), 7.99 (dd, J=5.2, 2.0 Hz, 1H), 7.49 (d, J=8.4 Hz, 2H), 7.43-7.37 (m, 3H), 7.17 (dd, J=7.6, 1.6 Hz, 1H), 7.01 (br s, 2H), 6.37 (dd, J=7.6, 4.8 Hz, 1H), 3.70-3.64 (m, 4H), 3.62 (s, 2H), 2.55-2.51 (m, 4H).

### Example 20: 2-(4-(4-(2-(2-aminopyridin-3-yl)-3H-imidazo[4,5-b]pyridin-3-yl)benzyl)piperazin-1-yl)pyrimidine-4-carbonitrile

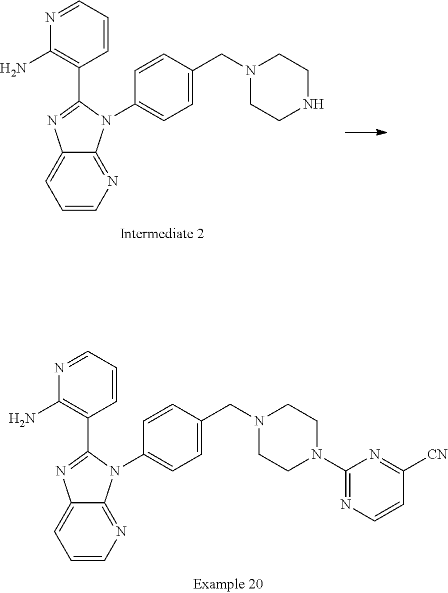 [SMILES: Nc1ncccc1-c1nc2cccnc2n1-c1ccc(CN2CCNCC2)cc1 ; N#Cc1ccnc(N2CCN(Cc3ccc(-n4c(-c5cccnc5N)nc5cccnc54)cc3)CC2)n1]

Following the general procedure of Example 17, the reaction of Intermediate 2 (200 mg, 519 μmol) with 2-chloropyrimidine-4-carbonitrile (144 mg, 1.03 mmol) was carried out. After purified by prep-HPLC (column: Waters xbridge 150*25 mm 10 μm; mobile phase: [water (NH₄HCO₃)-ACN]; B %: 27%-57%, 8 min), 2-(4-(4-(2-(2-aminopyridin-3-yl)-3H-imidazo[4,5-b]pyridin-3-yl)benzyl)piperazin-1-yl)pyrimidine-4-carbonitrile (Example 20, 49.3 mg, yield: 19%) was obtained as a yellow solid. MS: m/z=489.3 [M+H]⁺. ¹H NMR (400 MHz, Dimethylsulfoxide-d₆) δ 8.64 (d, J=4.8 Hz, 1H), 8.33 (dd, J=4.8, 1.6 Hz, 1H), 8.21 (dd, J=8.0, 1.6 Hz, 1H), 7.99 (dd, J=4.8, 2.0 Hz, 1H), 7.48 (d, J=8.4 Hz, 2H), 7.42-7.36 (m, 3H), 7.20-7.12 (m, 2H), 7.00 (br s, 2H), 6.37 (dd, J=7.6, 4.8 Hz, 1H), 3.82-3.74 (m, 4H), 3.61 (s, 2H), 2.49-2.43 (m, 4H).

### Example 21: 6-(4-(4-(2-(2-Aminopyridin-3-yl)-3H-imidazo[4,5-b]pyridin-3-yl)benzyl)piperazin-1-yl)pyrimidine-4-carbonitrile

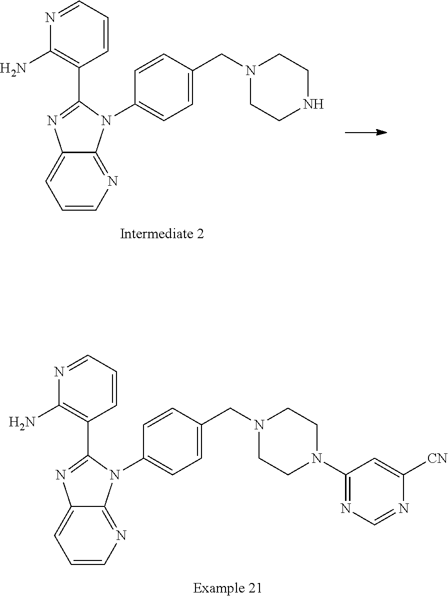 [SMILES: Nc1ncccc1-c1nc2cccnc2n1-c1ccc(CN2CCNCC2)cc1 ; N#Cc1cc(N2CCN(Cc3ccc(-n4c(-c5cccnc5N)nc5cccnc54)cc3)CC2)ncn1]

Following the general procedure of Example 17, the reaction of Intermediate 2 (200 mg, 519 μmol) with 6-chloropyrimidine-4-carbonitrile (144 mg, 1.03 mmol) was carried out. After purified by prep-HPLC (column: Waters xbridge 150*25 mm 10 μm; mobile phase: [water (NH₄HCO₃)-ACN]; B %: 27%-57%, 8 min), 6-(4-(4-(2-(2-aminopyridin-3-yl)-3H-imidazo[4,5-b]pyridin-3-yl)benzyl)piperazin-1-yl)pyrimidine-4-carbonitrile (Example 21, 11 mg, yield: 4.3%) was obtained as a brown solid. MS: m/z=489.3 [M+H]⁺. ¹H NMR (400 MHz, Dimethylsulfoxide-d₆) δ 8.57 (s, 1H), 8.33 (d, J=4.8 Hz, 1H), 8.20 (d, J=8.0 Hz, 1H), 8.02-7.94 (m, 1H), 7.56 (s, 1H), 7.48 (d, J=8.0 Hz, 2H), 7.43-7.37 (m, 3H), 7.16 (dd, J=7.6, 1.2 Hz, 1H), 7.01 (br s, 2H), 6.36 (dd, J=7.6, 4.8 Hz, 1H), 3.98-3.68 (m, 4H), 3.64 (s, 2H), 2.58-2.52 (m, 4H).

### Example 22: 6-(4-(4-(2-(2-Aminopyridin-3-yl)-3H-imidazo[4,5-h]pyridin-3-yl)benzyl)piperazin-1-yl)pyridazine-4-carbonitrile

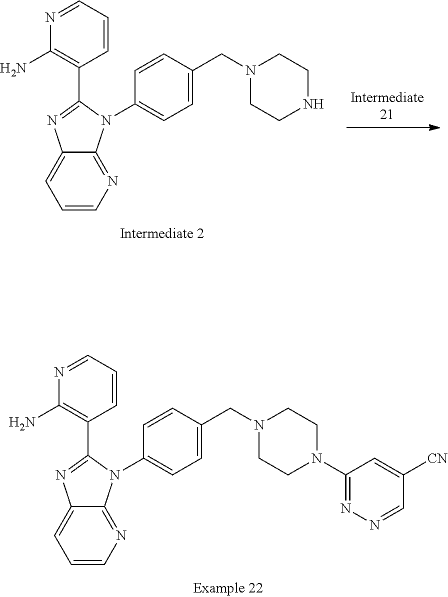 [SMILES: Nc1ncccc1-c1nc2cccnc2n1-c1ccc(CN2CCNCC2)cc1 ; N#Cc1cnnc(N2CCN(Cc3ccc(-n4c(-c5cccnc5N)nc5cccnc54)cc3)CC2)c1]

Following the general procedure of Example 17, the reaction of Intermediate 2 (24 mg, 63 μmol) with Intermediate 21 (9 mg, 63 μmol) was carried out. After purified by prep-HPLC (column: Waters xbridge 150*25 mm 10 μm; mobile phase: [water (NH₄HCO₃)-ACN]; B %: 22%-52%, 10 min), 6-(4-(4-(2-(2-aminopyridin-3-yl)-3H-imidazo[4,5-b]pyridin-3-yl)benzyl)piperazin-1-yl)pyridazine-4-carbonitrile (Example 22, 8.5 mg, yield: 28%) was obtained as a light-gray solid. MS: m/z=489.1 [M+H]⁺. ¹H NMR (400 MHz, Dimethylsulfoxide-d₆) δ 8.83 (d, J=1.2 Hz, 1H), 8.33 (dd, J=4.8, 1.2 Hz, 1H), 8.20 (dd, J=8.0, 1.2 Hz, 1H), 7.99 (dd, J=4.8, 1.6 Hz, 1H), 7.86 (d, J=1.2 Hz, 1H), 7.49 (d, J=8.4 Hz, 2H), 7.42-7.36 (m, 3H), 7.16 (dd, J=7.8, 1.6 Hz, 1H), 7.00 (br s, 2H), 6.36 (dd, J=7.6, 4.8 Hz, 1H), 3.75-3.67 (m, 4H), 3.62 (s, 2H), 2.55-2.52 (m, 4H).

### Example 23: N-(1-(4-(2-(2-aminopyridin-3-yl)-3H-imidazo[4,5-b]pyridin-3-yl)benzyl)piperidin-4-yl)-2-cyanoisonicotinamide

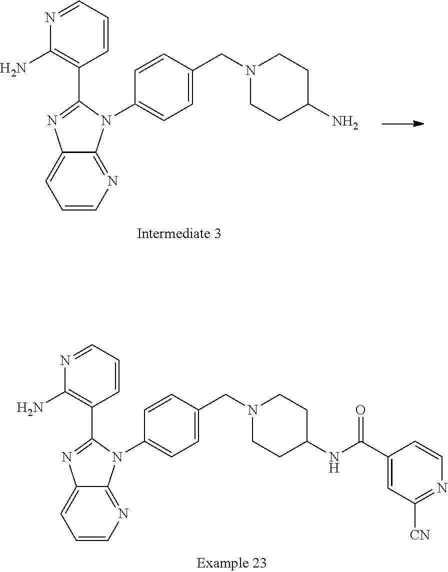 [SMILES: Nc1ncccc1-c1nc2cccnc2n1-c1ccc(CN2CCC(N)CC2)cc1 ; N#Cc1cc(C(=O)NC2CCN(Cc3ccc(-n4c(-c5cccnc5N)nc5cccnc54)cc3)CC2)ccn1]

To a solution of Intermediate 3 (200 mg, 500 μmol) in DMF (3 mL) was added HATU (381 mg, 1.00 mmol), 2-cyanopyridine-4-carboxylic acid (89 mg, 600 μmol) and DIEA (324 mg, 2.50 mmol). The mixture was stirred at 25° C. for 16 hr. The reaction mixture was poured into H₂O (5 mL) and extracted with EtOAc (10 mL×2). The combined organic layers were concentrated under reduced pressure to give a residue. The crude product was purified by prep-HPLC (column: Waters xbridge 150*25 mm 10 μm; mobile phase: [water (NH₄HCO₃)-ACN]; B %: 21%-51%, 11 min), N-(1-(4-(2-(2-aminopyridin-3-yl)-3H-imidazo[4,5-b]pyridin-3-yl)benzyl)piperidin-4-yl)-2-cyanoisonicotinamide (Example 23, 76 mg, yield: 29%) was obtained as a light-yellow solid. MS: m/z=530.2 [M+H]⁺. ¹H NMR (400 MHz, Dimethylsulfoxide-d₆) δ 8.90 (d, J=5.2 Hz, 1H), 8.73 (d, J=7.6 Hz, 1H), 8.37 (s, 1H), 8.33 (d, J=4.4 Hz, 1H), 8.20 (d, J=8.0 Hz, 1H), 8.07 (dd, J=4.8, 1.2 Hz, 1H), 7.99 (dd, J=4.8, 1.6 Hz, 1H), 7.45 (d, J=8.0 Hz, 2H), 7.41-7.37 (m, 3H), 7.18-7.15 (m, 1H), 7.01 (br s, 2H), 6.37 (dd, J=7.6, 4.8 Hz, 1H), 3.87-3.76 (m, 1H), 3.57 (s, 2H), 2.86 (d, J=11.6 Hz, 2H), 2.12-2.07 (m, 2H), 1.88-1.80 (m, 2H), 1.66-1.55 (m, 2H).

### Example 24: N-(1-(4-(2-(2-Aminopyridin-3-yl)-3H-imidazo[4,5-b]pyridin-3-yl)benzyl)piperidin-4-yl)-6-cyanonicotinamide

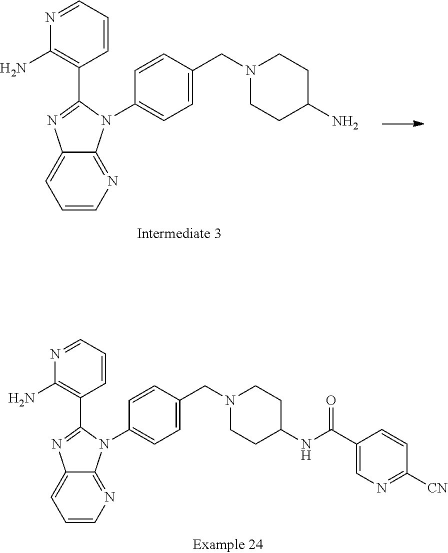 [SMILES: Nc1ncccc1-c1nc2cccnc2n1-c1ccc(CN2CCC(N)CC2)cc1 ; N#Cc1ccc(C(=O)NC2CCN(Cc3ccc(-n4c(-c5cccnc5N)nc5cccnc54)cc3)CC2)cn1]

Following the general procedure of Example 23, the reaction of Intermediate 3 (200 mg, 500 μmol) with 6-cyanopyridine-3-carboxylic acid (88.9 mg, 601 μmol) was carried out. After purified by prep-HPLC (column: Phenomenex C18 250*50 mm*10 μm; mobile phase: [water (NH₄HCO₃)-ACN]; B %: 20%-50%, 8 min), N-(1-(4-(2-(2-aminopyridin-3-yl)-3H-imidazo[4,5-b]pyridin-3-yl)benzyl)piperidin-4-yl)-6-cyanonicotinamide (Example 24, 74 mg, yield: 28%) was obtained as an off-white solid. MS: m/z=530.2 [M+H]⁺. ¹H NMR (400 MHz, Dimethylsulfoxide-d₆) δ 9.10 (s, 1H), 8.72 (d, J=7.6 Hz, 1H), 8.39 (dd, J=8.0, 2.0 Hz, 1H), 8.33 (dd, J=8.4, 1.2 Hz, 1H), 8.21 (dd, J=8.0, 1.2 Hz, 1H), 8.17 (dd, J=8.0 Hz, 1H), 7.99 (dd, J=8.4, 1.6 Hz, 1H), 7.45 (d, J=8.4 Hz, 2H), 7.42-7.36 (m, 3H), 7.16 (dd, J=7.6, 2.0 Hz, 1H), 7.01 (br s, 2H), 6.37 (dd, J=7.6, 4.8 Hz, 1H), 3.88-3.76 (m, 1H), 3.57 (s, 2H), 2.86 (d, J=11.6 Hz, 2H), 2.09 (t, J=11.6 Hz, 2H), 1.85 (d, J=10.0 Hz, 2H), 1.66-1.56 (m, 2H).

### Example 25: N-(1-(4-(2-(2-Aminopyridin-3-yl)-3H-imidazo[4,5-b]pyridin-3-yl)benzyl)piperidin-4-yl)-5-cyanopicolinamide

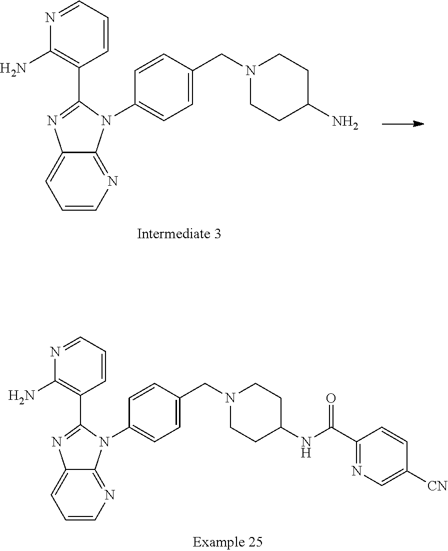 [SMILES: Nc1ncccc1-c1nc2cccnc2n1-c1ccc(CN2CCC(N)CC2)cc1 ; N#Cc1ccc(C(=O)NC2CCN(Cc3ccc(-n4c(-c5cccnc5N)nc5cccnc54)cc3)CC2)nc1]

Following the general procedure of Example 23, the reaction of Intermediate 3 (200 mg, 500 μmol) with 5-cyanopyridine-2-carboxylic acid (89 mg, 600 μmol) was carried out. After purified by prep-HPLC (column: Phenomenex luna C18 150*25 mm*10 μm. mobile phase: [water (NH₄HCO₃)-ACN]; B %: 22%-52%, 8 min), N-(1-(4-(2-(2-aminopyridin-3-yl)-3H-imidazo[4,5-b]pyridin-3-yl)benzyl)piperidin-4-yl)-5-cyanopicolinamide (Example 25, 12.7 mg, yield: 4.8%) was obtained as an off-white solid. MS: m/z=530.1 [M+H]⁺. ¹H NMR (400 MHz, Dimethylsulfoxide-d₆) δ 9.10 (d, J=1.6 Hz, 1H), 8.80 (d, J=8.4 Hz, 1H), 8.51 (dd, J=8.0, 2.0 Hz, 1H), 8.33 (dd, J=4.8, 1.2 Hz, 1H), 8.24-8.13 (m, 2H), 7.99 (dd, J=4.8, 1.6 Hz, 1H), 7.45 (d, J=8.4 Hz, 2H), 7.41-7.35 (m, 3H), 7.17 (dd, J=7.6, 1.6 Hz, 1H), 7.01 (br s, 2H), 6.37 (dd, J=7.6, 4.8 Hz, 1H), 3.88-3.78 (m, 1H), 3.56 (s, 2H), 2.84 (d, J=12.0 Hz, 2H), 2.15-2.05 (m, 2H), 1.80-1.70 (m, 4H).

### Example 26: N-(1-(4-(2-(2-aminopyridin-3-yl)-3H-imidazo[4,5-b]pyridin-3-yl)benzyl)piperidin-4-yl)-5-cyanonicotinamide

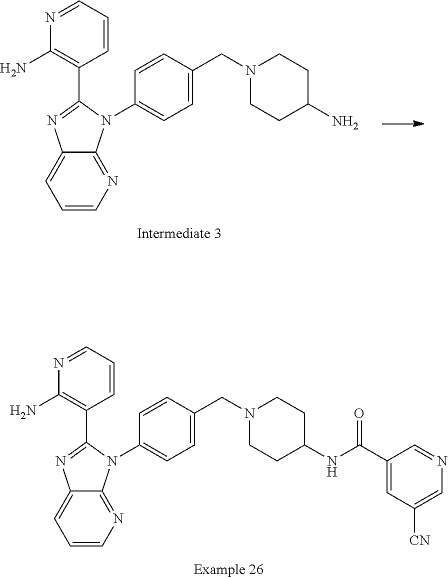 [SMILES: Nc1ncccc1-c1nc2cccnc2n1-c1ccc(CN2CCC(N)CC2)cc1 ; N#Cc1cncc(C(=O)NC2CCN(Cc3ccc(-n4c(-c5cccnc5N)nc5cccnc54)cc3)CC2)c1]

Following the general procedure of Example 23, the reaction of Intermediate 3 (200 mg, 500 μmol) with 5-cyanopyridine-3-carboxylic acid (89 mg, 600 μmol) was carried out. After purified by prep-HPLC (column: Phenomenex Luna C18 150*25 mm*10 μm; mobile phase: [water (NH₄HCO₃)-ACN]; B %: 18%-48%, 8 min), N-(1-(4-(2-(2-aminopyridin-3-yl)-3H-imidazo[4,5-b]pyridin-3-yl)benzyl)piperidin-4-yl)-5-cyanonicotinamide (Example 26, 26.8 mg, yield: 10.1%) was obtained as a yellow solid. MS: m/z=530.2 [M+H]⁺. ¹H NMR (400 MHz, Dimethylsulfoxide-d₆) δ 9.21 (d, J=2.0 Hz, 1H), 9.16 (d, J=2.0 Hz, 1H), 8.70-8.58 (m, 2H), 8.33 (dd, J=4.8, 1.2 Hz, 1H), 8.21 (dd, J=8.0, 1.2 Hz, 1H), 7.99 (dd, J=4.8, 1.6 Hz, 1H), 7.46 (d, J=8.4 Hz, 2H), 7.43-7.33 (m, 3H), 7.17 (dd, J=7.6, 1.6 Hz, 1H), 7.01 (br s, 2H), 6.37 (dd, J=7.6, 4.8 Hz, 1H), 3.87-3.75 (m, 1H), 3.57 (s, 2H), 2.86 (d, J=11.6 Hz, 2H), 2.10 (t, J=11.6 Hz, 2H), 1.89-1.80 (m, 2H), 1.67-1.54 (m, 2H).

### Example 27: N-(1-(4-(2-(2-Aminopyridin-3-yl)-3H-imidazo[4,5-b]pyridin-3-yl)benzyl)piperidin-4-yl)-2-cyanonicotinamide

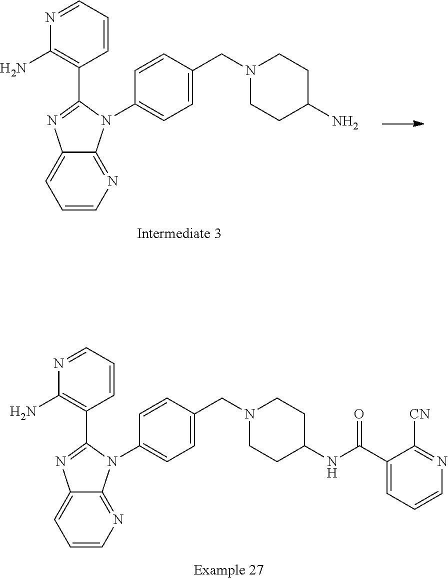 [SMILES: Nc1ncccc1-c1nc2cccnc2n1-c1ccc(CN2CCC(N)CC2)cc1 ; N#Cc1ncccc1C(=O)NC1CCN(Cc2ccc(-n3c(-c4cccnc4N)nc4cccnc43)cc2)CC1]

Following the general procedure of Example 23, the reaction of Intermediate 3 (200 mg, 500 μmol) 2-cyanopyridine-3-carboxylic acid (89 mg, 600 μmol) was carried out. After purified by prep-HPLC (column: Waters xbridge 150*25 mm 10 μm; mobile phase: [water (NH₄HCO₃)-ACN]; B %: 16%-46%, 8 min), N-(1-(4-(2-(2-aminopyridin-3-yl)-3H-imidazo[4,5-b]pyridin-3-yl)benzyl)piperidin-4-yl)-2-cyanonicotinamide (Example 27, 19.8 mg, yield: 7.5%) was obtained as a yellow solid. MS: m/z=530.3 [M+H]⁺. ¹H NMR (400 MHz, Dimethylsulfoxide-d₆) δ 9.88 (s, 1H), 8.92 (dd, J=4.8, 1.6 Hz, 1H), 8.33 (dd, J=4.8, 1.2 Hz, 1H), 8.26-8.01 (m, 2H), 7.98 (dd, J=4.8, 1.6 Hz, 1H), 7.73 (dd, 1=7.6, 4.8 Hz, 1H), 7.48 (d, J=8.0 Hz, 2H), 7.42-7.36 (m, 3H), 7.17 (dd, J=7.6, 1.8 Hz, 1H), 7.02 (br s, 2H), 6.37 (dd, J=7.6, 4.8 Hz, 1H), 4.36-4.24 (m, 1H), 3.59 (s, 2H), 2.99-2.89 (m, 2H), 2.61-2.56 (m, 2H), 2.14-2.03 (m, 2H), 1.67 (d, J=11.6 Hz, 2H).

### Example 28: N-(1-(4-(2-(2-Aminopyridin-3-yl)-3H-imidazo[4,5-b]pyridin-3-yl)benzyl)piperidin-4-yl)-3-cyanopicolinamide

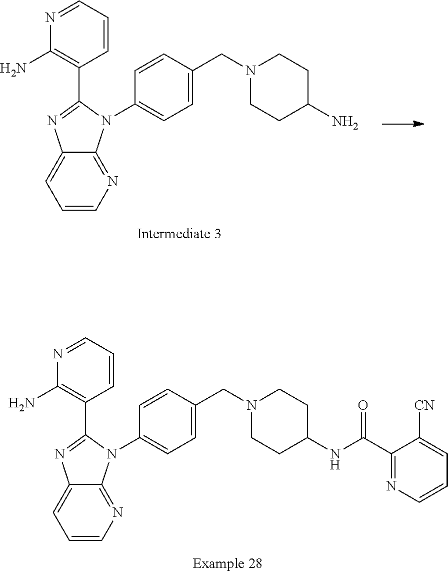 [SMILES: Nc1ncccc1-c1nc2cccnc2n1-c1ccc(CN2CCC(N)CC2)cc1 ; N#Cc1cccnc1C(=O)NC1CCN(Cc2ccc(-n3c(-c4cccnc4N)nc4cccnc43)cc2)CC1]

Following the general procedure of Example 23, the reaction of 3-(3-(4-((4-aminopiperidin-1-yl)methyl)phenyl)-3H-imidazo[4,5-b]pyridin-2-yl)pyridin-2-amine (200 mg, 500 μmol) with 3-cyanopyridine-2-carboxylic acid (89 mg, 600 μmol) was carried out. After purified by prep-HPLC (column: Waters xbridge 150*25 mm 10 μm; mobile phase: [water (NH₄HCO₃)-ACN]; B %: 18%-48%, 8 min), N-(1-(4-(2-(2-aminopyridin-3-yl)-3H-imidazo[4,5-b]pyridin-3-yl)benzyl)piperidin-4-yl)-3-cyanopicolinamide (Example 28, 40.7 mg, yield: 15%) was obtained as a pink solid. MS: m/z=529.9 [M+H]⁺. ¹H NMR (400 MHz, Chloroform-d) δ 8.95-8.90 (m, 1H), 8.87-8.67 (m, 1H), 8.40 (dd, J=4.8, 1.2 Hz, 1H), 8.10 (dd, 1=8.0, 1.2 Hz, 1H), 8.06-7.85 (m, 2H), 7.63-7.50 (m, 3H), 7.34 (d, J=8.4 Hz, 2H), 7.32-7.29 (m, 1H), 7.10 (dd, J=7.6, 1.6 Hz, 1H), 6.62 (br s, 2H), 6.35 (dd, J=7.6, 4.8 Hz, 1H), 4.58-4.44 (m, 1H), 3.63 (s, 2H), 3.08 (d, J=11.2 Hz, 2H), 2.96-2.68 (m, 2H), 2.26-2.16 (m, 2H), 1.77-1.73 (m, 2H).

### Example 29: N-(1-(4-(2-(2-Aminopyridin-3-yl)-3H-imidazo[4,5-b]pyridin-3-yl)benzyl)piperidin-4-yl)-4-cyanonicotinamide

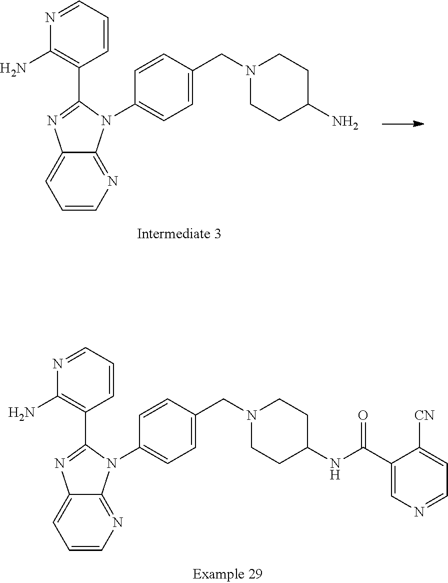 [SMILES: Nc1ncccc1-c1nc2cccnc2n1-c1ccc(CN2CCC(N)CC2)cc1 ; N#Cc1ccncc1C(=O)NC1CCN(Cc2ccc(-n3c(-c4cccnc4N)nc4cccnc43)cc2)CC1]

Following the general procedure of Example 23, the reaction of Intermediate 3 (200 mg, 500 μmol) with 4-cyanopyridine-3-carboxylic acid (89 mg, 600 μmol) was carried out. After purified by prep-HPLC (column: Phenomenex C18 150*25 mm*10 μm; mobile phase: [water (NH₄HCO₃)-ACN]; B %: 14%-44%, 8 min), N-(1-(4-(2-(2-aminopyridin-3-yl)-3H-imidazo[4,5-b]pyridin-3-yl)benzyl)piperidin-4-yl)-4-cyanonicotinamide (Example 29, 40.8 mg, yield: 15%) was obtained as a yellow solid. MS: m/z=530.1 [M+H]⁺. ¹H NMR (400 MHz, Dimethylsulfoxide-d₆) δ 10.59 (s, 1H), 9.05-8.97 (m, 2H), 8.33 (dd, J=4.8, 1.2 Hz, 1H), 8.21 (dd, J=8.0, 1.2 Hz, 1H), 8.15 (d, J=5.2 Hz, 1H), 7.99 (dd, J=4.8, 1.6 Hz, 1H), 7.48 (d, J=8.4 Hz, 2H), 7.41-7.35 (m, 3H), 7.17 (dd, J=7.6, 1.6 Hz, 1H), 7.01 (br s, 2H), 6.38 (dd, J=4.6, 4.8 Hz, 1H), 4.35-4.22 (m, 1H), 3.59 (s, 2H), 2.96 (d, J=11.2 Hz, 2H), 2.64-2.53 (m, 2H), 2.08 (t, J=11.2 Hz, 2H), 1.63 (d, J=9.6 Hz, 2H).

### Example 30: N-(1-(4-(2-(2-Aminopyridin-3-yl)-3H-imidazo[4,5-h]pyridin-3-yl)benzyl)piperidin-4-yl)-6-cyanopicolinamide

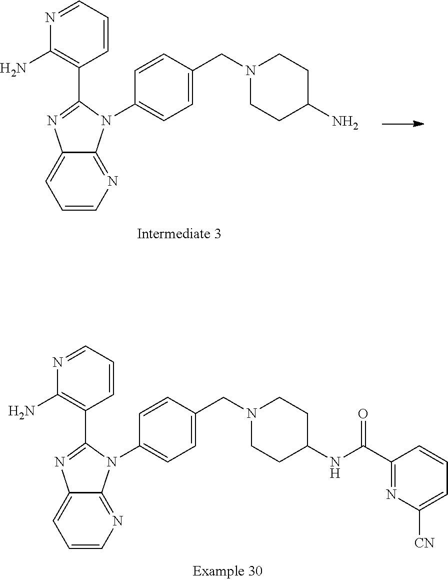 [SMILES: Nc1ncccc1-c1nc2cccnc2n1-c1ccc(CN2CCC(N)CC2)cc1 ; N#Cc1cccc(C(=O)NC2CCN(Cc3ccc(-n4c(-c5cccnc5N)nc5cccnc54)cc3)CC2)n1]

Following the general procedure of Example 23, the reaction of Intermediate 3 (200 mg, 500 μmol) with, 6-cyanopyridine-2-carboxylic acid (89 mg, 600 μmol) was carried out. After purified by prep-HPLC (column: Phenomenex luna C18 150*25 mm*10 μm; mobile phase: [water (NH₄HCO₃)-ACN]; B %: 22%-52%, 8 min), N-(1-(4-(2-(2-aminopyridin-3-yl)-3H-imidazo[4,5-b]pyridin-3-yl)benzyl)piperidin-4-yl)-6-cyanopicolinamide (Example 30, 36.7 mg, yield: 14%) was obtained as a yellow solid. MS: m/z=530.1 [M+H]⁺. ¹H NMR (400 MHz, Dimethylsulfoxide-d₆) δ 8.71 (d, J=8.4 Hz, 1H), 8.33 (dd, J=4.8, 1.2 Hz, 1H), 8.30-8.26 (m, 1H), 8.24-8.21 (m, 2H), 8.20-8.06 (m, 1H), 7.99 (dd, J=4.8, 1.6 Hz, 1H), 7.46 (d, J=8.4 Hz, 2H), 7.42-7.36 (m, 3H), 7.17 (dd, J=7.6, 1.6 Hz, 1H), 7.01 (br s, 2H), 6.37 (dd, J=7.2, 4.8 Hz, 1H), 3.91-3.77 (m, 1H), 3.57 (s, 2H), 2.86 (d, J=11.2 Hz, 2H), 2.16-2.05 (m, 2H), 1.81-1.70 (m, 4H).

### Example 31: N-(1-(4-(2-(2-Aminopyridin-3-yl)-3H-imidazo[4,5-b]pyridin-3-yl)benzyl)piperidin-4-yl)-4-cyanopicolinamide

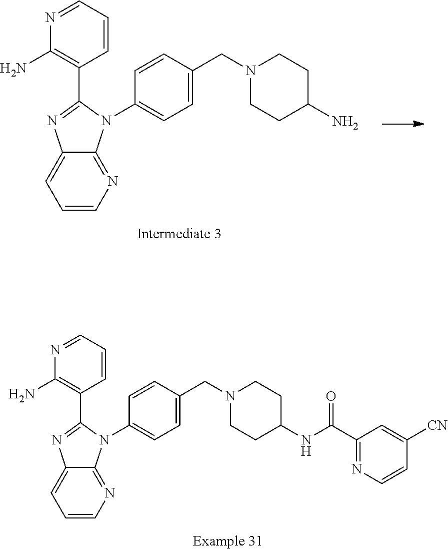 [SMILES: Nc1ncccc1-c1nc2cccnc2n1-c1ccc(CN2CCC(N)CC2)cc1 ; N#Cc1ccnc(C(=O)NC2CCN(Cc3ccc(-n4c(-c5cccnc5N)nc5cccnc54)cc3)CC2)c1]

Following the general procedure of Example 138, the reaction of Intermediate 3 (150 mg, 375 μmol) with 4-cyanopyridine-2-carboxylic acid (55.6 mg, 375 μmol) was carried out. After purified by prep-HPLC (column: Waters xbridge 150×25 mm 10 μm; mobile phase: [water(NH₄HCO₃)-ACN]; B %: 23%-53%, 8 min), N-(1-(4-(2-(2-aminopyridin-3-yl)-3H-imidazo[4,5-b]pyridin-3-yl)benzyl)piperidin-4-yl)-4-cyanopicolinamide (Example 31, 17.7 mg, yield: 9%) was obtained as a yellow solid. MS: m/z=530.1 [M+H]⁺. ¹H NMR (400 MHz, Chloroform-d) 8.74 (d, J=5.2 Hz, 1H), 8.44-8.37 (m, 2H), 8.10 (dd, J=8.0, 1.2 Hz, 1H), 8.05 (dd, =4.8, 1.6 Hz, 1H), 7.91 (d, =8.0 Hz, 1H), 7.67 (dd, J=4.8, 1.6 Hz, 1H), 7.56 (d, J=8.0 Hz, 2H), 7.36 (d, J=8.4 Hz, 2H), 7.32 (dd, J=8.0, 4.8 Hz, 1H), 7.07 (dd, J=8.0 1.6 Hz, 1H), 6.64 (br s, 2H), 6.34 (dd, J=8.0, 4.8 Hz, 1H), 4.12-4.03 (m, 1H), 3.70 (s, 2H), 3.07-2.97 (m, 2H), 2.39-2.30 (m, 2H), 2.10-2.05 (m, 2H), 1.84-1.78 (m, 2H).

### Example 32: N-(1-(4-(2-(2-Aminopyridin-3-yl)-3H-imidazo[4,5-h]pyridin-3-yl)benzyl)piperidin-4-yl)-2-cyanopyrimidine-4-carboxamide

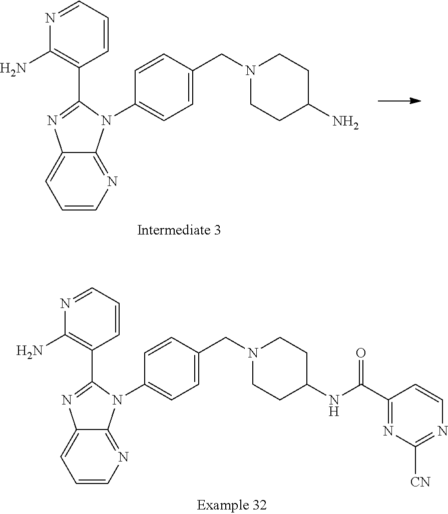 [SMILES: Nc1ncccc1-c1nc2cccnc2n1-c1ccc(CN2CCC(N)CC2)cc1 ; N#Cc1nccc(C(=O)NC2CCN(Cc3ccc(-n4c(-c5cccnc5N)nc5cccnc54)cc3)CC2)n1]

To a solution of Intermediate 3 (130 mg, 325 μmol), 2-cyanopyrimidine-4-carboxylic acid (64.7 mg, 391 μmol), EDCI (81.1 mg, 423 μmol) and HOBt (57.2 mg, 423 μmol) in CH₂Cl₂ (2 mL) was added DIEA (210 mg, 1.63 mmol). The mixture was stirred at 25° C. for 14 hr. The reaction mixture was diluted with H₂O (5 mL) and extracted with CH₂Cl₂ (10 mL×3). The combined organic layers were washed with brine (10 mL), dried over Na₂SO₄, filtered, and concentrated under reduced pressure to give a residue. The crude product was purified by prep-HPLC (column: Waters xbridge 150×25 mm 10 μm; mobile phase: [water (NH₄HCO₃)-ACN]; B %: 25%-55%, 15 min), N-(1-(4-(2-(2-aminopyridin-3-yl)-3H-imidazo[4,5-b]pyridin-3-yl)benzyl)piperidin-4-yl)-2-cyanopyrimidine-4-carboxamide (Example 32, 10.0 mg, yield: 5.8%) was obtained as a light-yellow solid. MS: m/z=531.0 [M+H]⁺. ¹H NMR (400 MHz, Dimethylsulfoxide-d₆) δ 9.22 (d, J=5.2 Hz, 1H), 9.04 (d, J=8.4 Hz, 1H), 8.36-8.30 (m, 1H), 8.26 (d, J=5.2 Hz, 1H), 8.22-8.19 (m, 1H), 7.99 (dd, J=4.8, 1.6 Hz, 1H), 7.48-7.36 (m, 5H), 7.19-7.15 (m, 1H), 7.01 (br s, 2H), 6.37 (dd, J=7.6, 4.8 Hz, 1H), 3.90-3.75 (m, 1H), 3.56 (s, 2H), 2.91-2.83 (m, 2H), 2.13-2.04 (m, 2H), 1.80-1.71 (m, 4H).

### Example 33: N-(1-(4-(2-(2-Aminopyridin-3-yl)-3H-imidazo[4,5-b]pyridin-3-yl)benzyl)piperidin-4-yl)-6-cyanopyrimidine-4-carboxamide

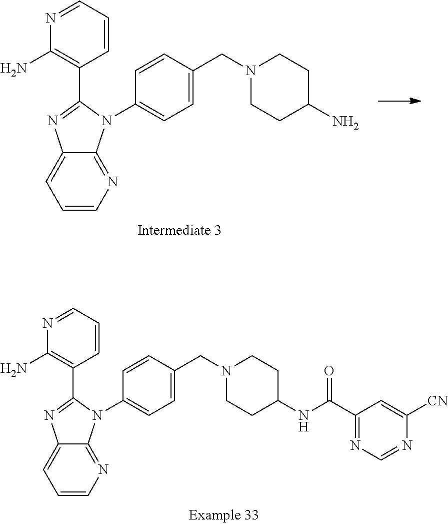 [SMILES: Nc1ncccc1-c1nc2cccnc2n1-c1ccc(CN2CCC(N)CC2)cc1 ; N#Cc1cc(C(=O)NC2CCN(Cc3ccc(-n4c(-c5cccnc5N)nc5cccnc54)cc3)CC2)ncn1]

Following the general procedure of Example 113, the reaction of Intermediate 3 (200 mg, 501 μmol) with 6-cyanopyrimidine-4-carboxylic acid (74.7 mg, 501 μmol) was carried out. After purified by prep-HPLC (column: Waters xbridge 150×25 mm 10 μm; mobile phase: [water (NH₄HCO₃)-ACN]; B %: 22%-5 2%, 8 min), N-(1-(4-(2-(2-aminopyridin-3-yl)-3H-imidazo[4,5-b]pyridin-3-yl)benzyl)piperidin-4-yl)-6-cyanopyrimidine-4-carboxamide (Example 33, 32.7 mg, yield: 12%) was obtained as a yellow solid. MS: m/z=531.1 [M+H]⁺. ¹H NMR (400 MHz, Dimethylsulfoxide-d₆) δ 9.53 (d, J=1.6 Hz, 1H), 9.05 (d, J=8.4 Hz, 1H), 8.53 (d, J=1.6 Hz, 1H), 8.33 (dd, J=4.4, 1.2 Hz, 1H), 8.20 (dd, J=8.0, 1.6 Hz, 1H), 7.99 (dd, J=4.8, 2.0 Hz, 1H), 7.45 (d, J=8.4 Hz, 2H), 7.41-7.36 (m, 3H), 7.17 (dd, J=7.6, 2.0 Hz, 1H), 7.00 (br s, 2H), 6.37 (dd, J=7.6, 4.8 Hz, 1H), 3.90-3.77 (m, 1H), 3.56 (s, 2H), 2.85 (d, J=11.6 Hz, 2H), 2.13-2.05 (m, 2H), 1.78-1.71 (m, 4H).

### Example 34: N-(1-(4-(2-(2-aminopyridin-3-yl)-3H-imidazo[4,5-b]pyridin-3-yl)benzyl)piperidin-4-yl)-2-cyanobenzo[d]thiazole-5-carboxamide

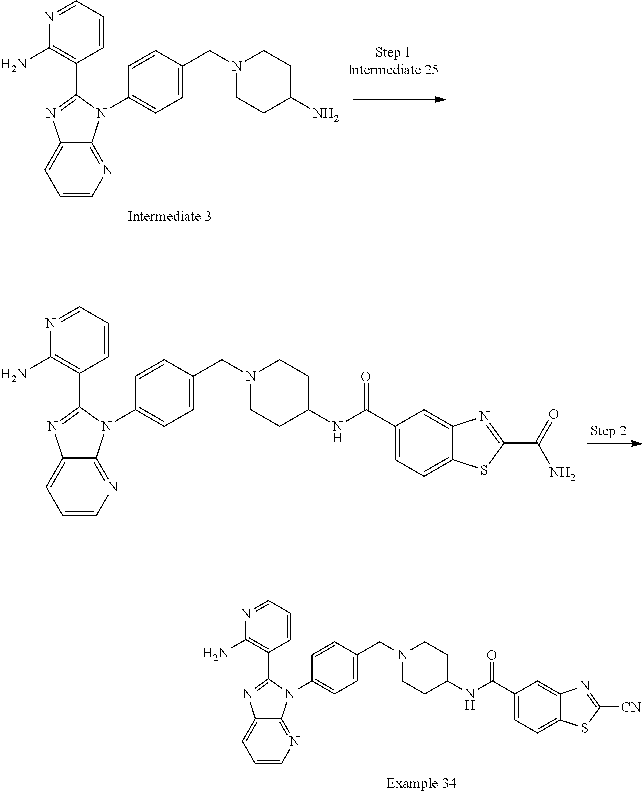 [SMILES: Nc1ncccc1-c1nc2cccnc2n1-c1ccc(CN2CCC(N)CC2)cc1 ; NC(=O)c1nc2cc(C(=O)NC3CCN(Cc4ccc(-n5c(-c6cccnc6N)nc6cccnc65)cc4)CC3)ccc2s1 ; N#Cc1nc2cc(C(=O)NC3CCN(Cc4ccc(-n5c(-c6cccnc6N)nc6cccnc65)cc4)CC3)ccc2s1]

### Step 1; N⁵-(1-(4-(2-(2-Aminopyridin-3-yl)-3H-imidazo[4,5-b]pyridin-3-yl)benzyl)piperidin-4-yl)benzo[d]thiazole-2,5-dicarboxamnide

Following the general procedure Example 173 step 1, the reaction of Intermediate 3 (300 mg, 751 μmol) with Intermediate 25 (200 mg, 901 μmol) was carried out. After purified by prep-HPLC (column: Phenomenex C18 150*25 mm*10 μm; mobile phase: [water (NH₄HCO₃)-ACN]; B %: 16%-46%, 8 min), N⁵-(1-(4-(2-(2-aminopyridin-3-yl)-3H-imidazo[4,5-b]pyridin-3-yl)benzyl)piperidin-4-yl)benzo[d]thiazole-2,5-dicarboxamide (28.3 mg, yield: 6.2%) was obtained as an off-white solid. MS: m/z=604.2 [M+H]⁺. ¹H NMR (400 MH-z, Dimethylsulfoxide-d₆) δ 8.58 (d, J=1.2 Hz, 1H), 8.53 (d, J=7.2 Hz, 1H), 8.46 (s, 1H), 8.34 (dd, J=4.8, 1.2 Hz, 1H), 8.29 (d, J=8.4 Hz, 1H), 8.21 (dd, J=7.6, 1.2 Hz, 1H), 8.15 (s, 1H), 8.03 (dd, J=8.4, 1.2 Hz, 1H), 7.99 (dd, J=4.8, 1.6 Hz, 1H), 7.47 (d, J=8.0 Hz, 2H), 7.42-7.37 (m, 3H), 7.17 (dd, J=7.6, 4.8 Hz, 1H), 7.01 (br s, 2H), 6.38 (dd, J=7.6, 4.8 Hz, 1H), 3.92-3.79 (in, 1H), 3.58 (s, 2H), 2.92-2.86 (m, 2H), 2.16-2.07 (m, 2H), 1.86 (d, J=10.4 Hz, 2H), 1.71-1.61 (m, 2H).

### Step 2: N⁵-(1-(4-(2-(2-aminopyridin-3-yl)-3H-imidazo[4,5-b]pyridin-3-yl)benzyl)piperidin-4-yl)benzo[d]thiazole-2,5-dicarboxamide

Following the general procedure of Example 173 step 2, the reaction of N-(1-(4-(2-(2-aminopyridin-3-yl)-3H-imidazo[4,5-b]pyridin-3-yl)benzyl)piperidin-4-yl)benzo[d]thiazole-2,5-dicarboxamide (50 mg, 45 μmol) with POCl₃ (173 mg, 1.0 mmol) was carried out. The crude product was purified by reversed-phase HPLC (0.1% NH₃HCO₃), N-(1-(4-(2-(2-aminopyridin-3-yl)-3H-imidazo[4,5-b]pyridin-3-yl)benzyl)piperidin-4-yl)-2-cyanobenzo[d]thiazole-5-carboxamide (Example 34, 4.2 mg, yield: 16%) was obtained as a light-green solid. MS: m/z=586.2 [M+H]⁺. ¹H NMR (400 MHz, Chlorform-d) δ 8.55 (s, 1H), 8.40 (d, J=4.4 Hz, 1H), 8.15-8.02 (m, 4H), 7.52 (d, J=7.6 Hz, 2H), 7.37-7.30 (m, 3H), 7.08 (d, J=8.0 Hz, 1H), 6.66 (br s, 2H), 6.38-6.30 (m, 1H), 6.17 (d, J=8.0 Hz, 1H), 4.17-4.03 (m, 1H), 3.63 (s, 2H), 3.01-2.87 (m, 2H), 2.28 (t, J=10.8 Hz, 2H), 2.11 (d, J=12.4 Hz, 2H), 1.65-1.61 (m, 2H).

### Example 35: N-(1-(4-(2-(2-Aminopyridin-3-yl)-3H-imidazo[4,5-b]pyridin-3-yl)benzyl)piperidin-4-yl)-2-cyano-5-fluorobenzo[d]thiazole-7-carboxamide

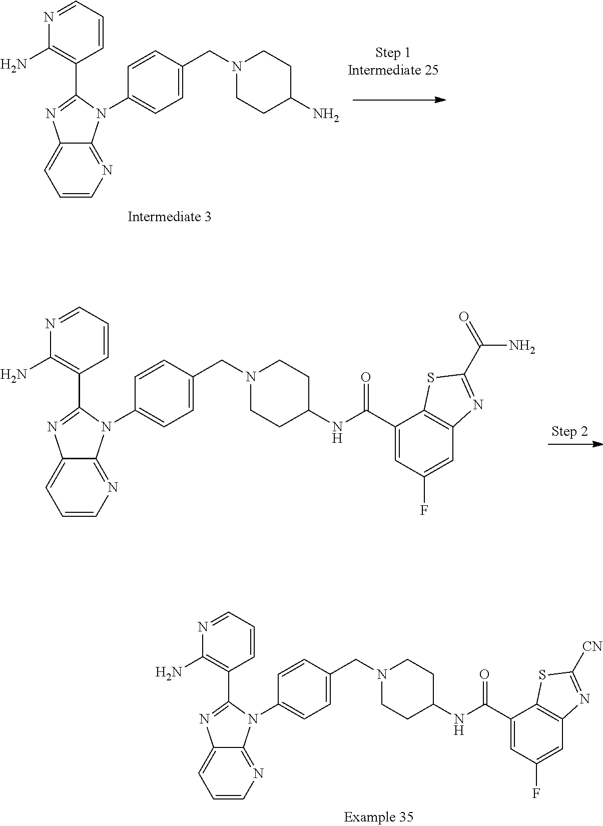 [SMILES: Nc1ncccc1-c1nc2cccnc2n1-c1ccc(CN2CCC(N)CC2)cc1 ; NC(=O)c1nc2cc(F)cc(C(=O)NC3CCN(Cc4ccc(-n5c(-c6cccnc6N)nc6cccnc65)cc4)CC3)c2s1 ; N#Cc1nc2cc(F)cc(C(=O)NC3CCN(Cc4ccc(-n5c(-c6cccnc6N)nc6cccnc65)cc4)CC3)c2s1]

### Step 1: N⁷-(1-(4-(2-(2-Aminopyridin-3-yl)-3H-imidazo[4,5-b]pyridin-3-yl)benzyl)piperidin-4-yl)-5-fluorobenzo[d]thiazole-2,7-dicarboxamide

Following the general procedure of Example 173 step 1, the reaction of Intermediate 3 (50 mg, 125 μmol) with Intermediate 26 (30 mg, 125 μmol) was carried out. After purified by prep-HPLC (column: Waters xbridge 150×25 mm 10 μm; mobile phase: [water (NH₄HCO₃)-ACN]; B %: 20%-50%, 11 min), N⁷-(1-(4-(2-(2-aminopyridin-3-yl)-3H-imidazo[4,5-b]pyridin-3-yl)benzyl)piperidin-4-yl)-5-fluorobenzo[d]thiazole-2,7-dicarboxamide (12.7 mg, yield. 15.2%) was obtained as a yellow lyophilized powder. MS: m/z=622.2 [M+H]⁺. ¹H NMR (400 MHz, Dimethylsulfoxide-d₆) δ 8.76 (d, J 4.8 Hz, 1H), 8.45 (s, 1H), 8.33 (dd, J 4.8, 1.2 Hz, 1H), 8.26 (dd, J=10.0, 2.0 Hz, 1H), 8.20 (dd, J=8.0, 1.6 Hz, 1H), 8.14 (dd, J=8.8, 2.0 Hz, 1H), 8.08 (s, 1H), 7.99 (dd, J=4.8, 1.6 Hz, 1H), 7.46 (d, J=8.0 Hz, 2H), 7.42-7.36 (m, 3H), 7.16 (dd, J=7.6, 2.0 Hz, 1H), 7.01 (br s, 2H), 6.38 (dd, J=7.6, 4.8 Hz, 1H), 3.93-3.83 (m, 1H), 3.58 (s, 2H), 2.92-2.83 (m, 2H), 2.15-2.09 (m, 2H), 1.89-1.85 (m, 2H), 1.72-1.60 (m, 2H). ¹⁹F NMR (400 MHz, Dimethylsulfoxide-d₆) δ −114.6.

### Step 2: N-(1-(4-(2-(2-Aminopyridin-3-yl)-3H-imidazo[4,5-b]pyridin-3-yl)benzyl)piperidin-4-yl)-2-cyano-5-fluorobenzo[d]thiazole-7-carboxamide

Following the general procedure of Example 173 step 2, the reaction of N⁷-(1-(4-(2-(2-aminopyridin-3-yl)-3H-imidazo[4,5-b]pyridin-3-yl)benzyl)piperidin-4-yl)-5-fluorobenzo[d]thiazole-2,7-dicarboxamide (30 mg, 48 μmol) with POCl₃ was carried out. After purified by prep-HPLC (column: Phenomenex C18 150×25 mm×10 μm; mobile phase: [water (NH₄HCO₃)-ACN]; B %: 36%-66%, 8 min), N-(1-(4-(2-(2-aminopyridin-3-yl)-3H-imidazo[4,5-b]pyridin-3-yl)benzyl)piperidin-4-yl)-2-cyano-5-fluorobenzo[d]thiazole-7-carboxamide (Example 35, 5 mg yield: 17%) was obtained as an off-white lyophilized powder. MS: m/z=604.3 [M+H]⁺. ¹H NMR (400 MHz, Dimethylsulfoxide-d₆) δ 8.96 (d, J=7.6 Hz, 1H), 8.47 (dd, J=9.6, 2.4 Hz, 1H), 8.41 (dd, J=8.8, 2.4 Hz, 1H), 8.33 (dd, J=4.8, 1.2 Hz, 1H), 8.21 (dd, J=8.0, 1.2 Hz, 1H), 7.99 (dd, J=4.8, 1.2 Hz, 1H), 7.47 (d, J=8.4 Hz, 2H), 7.42-7.36 (m, 3H), 7.17 (dd, J=7.6, 2.0 Hz, 1H), 7.01 (br s, 2H), 6.38 (dd, J=7.6, 4.8 Hz, 1H), 3.90-3.86 (m, 1H), 3.58 (s, 2H), 2.89 (d, J=11.2 Hz, 2H), 2.11 (t, J=11.2 Hz, 2H), 1.89-1.85 (m, 2H), 1.72-1.60 (m, 2H). ¹⁹F NMR (400 MHz, Dimethylsulfoxide-d₆) δ −113.1.

### Example 36: N-(1-(4-(2-(2-Aminopyridin-3-yl)-3H-imidazo[4,5-b]pyridin-3-yl)benzyl)piperidin-4-yl)-3-cyanoisonicotinamide

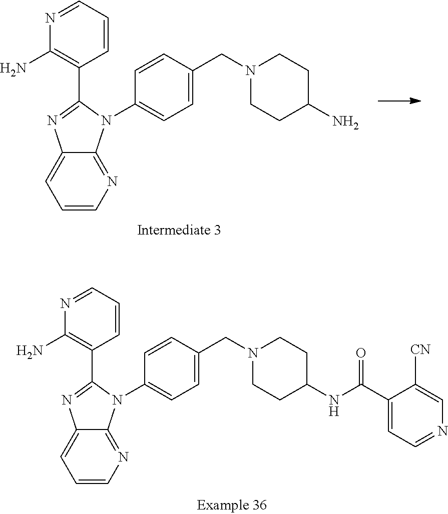 [SMILES: Nc1ncccc1-c1nc2cccnc2n1-c1ccc(CN2CCC(N)CC2)cc1 ; N#Cc1cnccc1C(=O)NC1CCN(Cc2ccc(-n3c(-c4cccnc4N)nc4cccnc43)cc2)CC1]

Following the general procedure of Example 23, the reaction of Intermediate 3 (150 mg, 375 μmol) with 3-cyanopyridine-4-carboxylic acid (67 mg, 451 μmol) was carried out. After purified by prep-HPLC (column: Waters xbridge 150*25 mm 10 μm; mobile phase: [water (NH₄HCO₃)-ACN]; B %: 18%-48%, 8 min), N-(1-(4-(2-(2-aminopyridin-3-yl)-3H-imidazo[4,5-b]pyridin-3-yl)benzyl)piperidin-4-yl)-3-cyanoisonicotinamide (Example 36, 38.6 mg, yield: 19%) was obtained as a pink solid. MS: m/z=530.2 [M+H]⁺. ¹H NMR (400 MHz, Dimethylsulfoxide-d₆) δ 10.52 (s, 1H), 9.40 (s, 1H), 8.94 (d, J=4.8 Hz, 1H), 8.38-8.28 (m, 1H), 8.20 (d, J=8.0 Hz, 1H) 8.03-7.92 (m, 1H), 7.77 (d, J=4.8 Hz, 1H), 7.48 (d, J=8.0 Hz, 2H), 7.44-7.36 (m, 3H), 7.22-7.14 (m, 1H), 7.01 (br s, 2H), 6.37 (dd, J=7.6, 4.8 Hz, 1H), 4.42-4.17 (m, 1H), 3.59 (s, 2H), 3.04-2.90 (m, 2H), 2.54 (br s, 2H), 2.14-2.02 (m, 2H), 1.70-1.55 (m, 2H).

### Example 37: N-(3-(2-(2-Aminopyridin-3-yl)-3-(4-((4-(2-cyanopyridin-4-yl)piperazin-1-yl)methyl)phenyl)-3H-imidazo[4,5-b]pyridin-5-yl)phenyl)acetamide

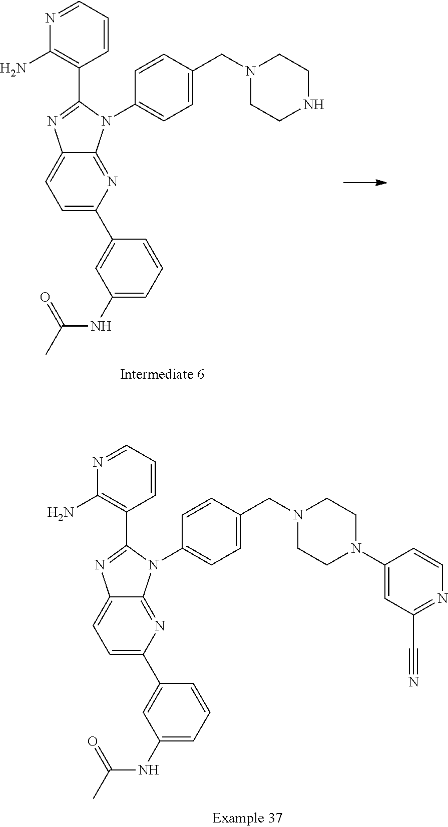 [SMILES: CC(=O)Nc1cccc(-c2ccc3nc(-c4cccnc4N)n(-c4ccc(CN5CCNCC5)cc4)c3n2)c1 ; CC(=O)Nc1cccc(-c2ccc3nc(-c4cccnc4N)n(-c4ccc(CN5CCN(c6ccnc(C#N)c6)CC5)cc4)c3n2)c1]

To a solution of Intermediate 6 (150 mg, 289 μmol), 4-fluoropicolinonitrile (38.8 mg, 318 μmol) in DMF (2 mL) was added DIEA (112 mg, 868 μmol). The mixture was degassed and purged with N₂ three times and stirred at 100° C. for 16 hr under N₂. The reaction mixture was filtered and concentrated under reduced pressure to give a residue. The residue was purified by prep-HPLC (column: Waters xbridge 150*25 mm 10 μm; mobile phase: [water (NH₄HCO₃)-ACN]; B %: 26%-56%, 8 min), N-(3-(2-(2-aminopyridin-3-yl)-3-(4-((4-(2-cyanopyridin-4-yl)piperazin-1-yl)methyl)phenyl)-3H-imidazo[4,5-b]pyridin-5-yl)phenyl)acetamide (Example 37, 155 mg, yield: 86%) was obtained as a yellow solid. MS: m/z=621.2 [M+H]⁺. ¹H NMR (400 MHz, Chloroform-d) δ 8.30 (d, J=6.0 Hz, 1H), 8.16-8.11 (m, 2H), 8.08 (dd, J=4.8, 1.2 Hz, 1H), 7.81 (d, J=8.0 Hz, 1H), 7.77 (d, J=7.6 Hz, 1H), 7.56 (d, J=8.0 Hz, 1H), 7.51 (d, J=8.0 Hz, 2H), 7.45-7.35 (m, 4H), 7.09 (d, J=8.0 Hz, 1H), 7.04 (d, J=1.6 Hz, 1H), 6.80-5.75 (m, 1H), 6.61 (br s, 2H), 6.37 (dd, J=7.6, 4.8 Hz, 1H), 3.65 (s, 2H), 3.49-3.42 (m, 4H), 2.59-2.52 (m, 4H), 2.05 (s, 3H).

### Example 38: N-(3-(2-(2-Aminopyridin-3-yl)-3-(4-((4-(6-cyanopyridin-3-yl)piperazin-1-yl)methyl)phenyl)-3H-imidazo[4,5-b]pyridin-5-yl)phenyl)acetamide

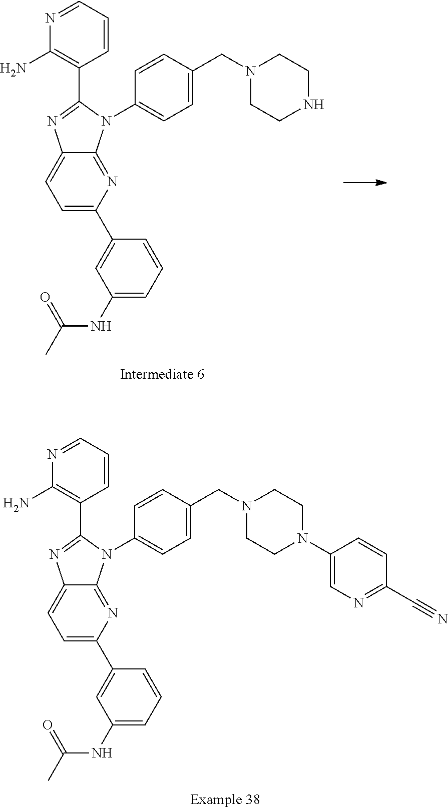 [SMILES: CC(=O)Nc1cccc(-c2ccc3nc(-c4cccnc4N)n(-c4ccc(CN5CCNCC5)cc4)c3n2)c1 ; CC(=O)Nc1cccc(-c2ccc3nc(-c4cccnc4N)n(-c4ccc(CN5CCN(c6ccc(C#N)nc6)CC5)cc4)c3n2)c1]

Following the general procedure of Example 37, the reaction of Intermediate 6 (150 mg, 289 μmol) with 5-fluoropicolinonitrile (38.8 mg, 318 μmol) was carried out. After purified by prep-HPLC (column: Phenomenex Luna C18 150*25 mm*10 μm; mobile phase: [water (NH₄HCO₃)-ACN]; B %: 35%-65%, 8 min), N-(3-(2-(2-aminopyridin-3-yl)-3-(4-((4-(6-cyanopyridin-3-yl)piperazin-1-yl)methyl)phenyl)-3H-imidazo[4,5-b]pyridin-5-yl)phenyl)acetamide (Example 38, 105 mg, yield: 58%) was obtained as a white solid. MS: m/z=621.2 [M+H]⁺. ¹H NMR (400 MHz, Dimethylsulfoxide-d₆) δ 10.05 (s, 1H), 8.43 (d, J=2.8 Hz, 1H), 8.28 (d, J=8.4 Hz, 1H), 8.16 (s, 1H), 8.00 (dd, J=4.8 Hz, 1H), 7.86 (d, J=8.4 Hz, 1H), 7.75 (d, J=8.8 Hz, 1H), 7.69-7.63 (m, 2H), 7.54-7.44 (m, 4H), 7.41-7.33 (m, 2H), 7.14 (dd, J=7.6, 1.6 Hz, 1H), 6.99 (br s, 2H), 6.37 (dd, J=7.6, 4.8 Hz, 1H), 3.65 (s, 2H), 3.49-3.42 (m, 4H), 2.59-2.52 (m, 4H), 2.05 (s, 3H).

### Example 39: N-(3-(2-(2-Aminopyridin-3-yl)-3-(4-((4-(6-cyanopyridin-2-yl)piperazin-1-yl)methyl)phenyl)-3H-imidazo[4,5-b]pyridin-5-yl)phenyl)acetamide

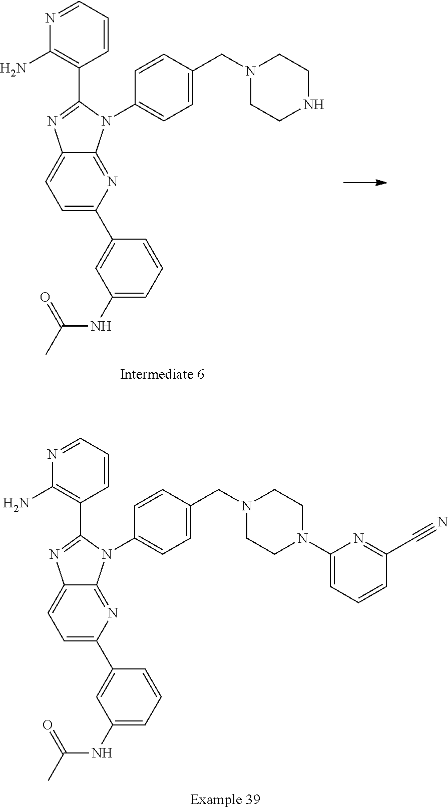 [SMILES: CC(=O)Nc1cccc(-c2ccc3nc(-c4cccnc4N)n(-c4ccc(CN5CCNCC5)cc4)c3n2)c1 ; CC(=O)Nc1cccc(-c2ccc3nc(-c4cccnc4N)n(-c4ccc(CN5CCN(c6cccc(C#N)n6)CC5)cc4)c3n2)c1]

Following the general procedure of Example 37, the reaction of Intermediate 6 (200 mg, 386 μmol) with 6-fluoropicolinonitrile (51.8 mg, 424 μmol) was carried out. After purified by prep-HPLC (column: Phenomenex C18 150*25 mm*10 μm; mobile phase: [water (NH₄HCO₃)-ACN]; B %: 40%-70%, 8 min), N-(3-(2-(2-aminopyridin-3-yl)-3-(4-((4-(6-cyanopyridin-2-yl)piperazin-1-yl)methyl)phenyl)-3H-imidazo[4,5-b]pyridin-5-yl)phenyl)acetamide (Example 39, 56 mg, yield: 23%) was obtained as a white solid. MS: m/z=621.2 [M+H]⁺. ¹H NMR (400 MHz, Dimethylsulfoxide-d₆) δ 10.04 (s, 1H), 8.28 (d, J=8.4 Hz, 1H), 8.8 (s, 1H), 8.00 (dd, J=4.8, 1.6 Hz, 1H), 7.87 (d, J=8.4 Hz, 1H), 7.73-7.61 (m, 3H), 7.55-7.43 (m, 4H), 7.38 (dd, J=8.0, 8.0 Hz, 1H), 7.23-7.12 (m, 3H), 7.00 (br s, 2H), 6.37 (dd, J=7.6, 4.8 Hz, 1H), 3.64 (s, 2H), 3.61-3.56 (m, 4H), 2.54-2.51 (m, 4H), 2.07 (s, 3H).

### Example 40: N-(3-(2-(2-aminopyridin-3-yl)-3-(4-((4-(6-cyanopyrazin-2-yl)piperazin-1-yl)methyl)phenyl)-3H-imidazo[4,5-b]pyri din-5-yl)phenyl)acetamide

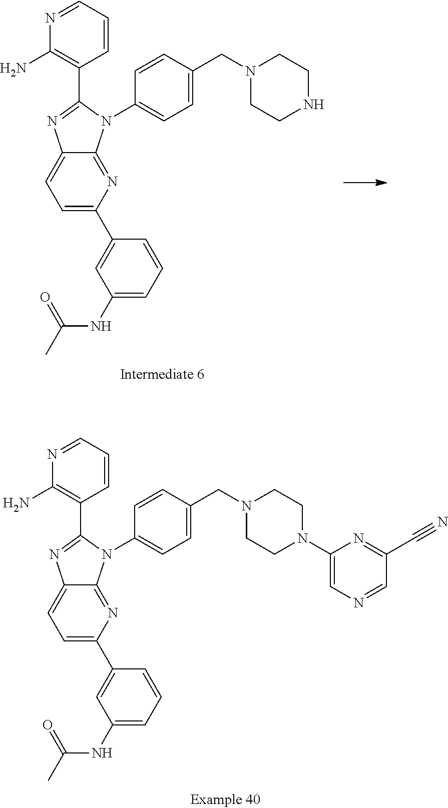 [SMILES: CC(=O)Nc1cccc(-c2ccc3nc(-c4cccnc4N)n(-c4ccc(CN5CCNCC5)cc4)c3n2)c1 ; CC(=O)Nc1cccc(-c2ccc3nc(-c4cccnc4N)n(-c4ccc(CN5CCN(c6cncc(C#N)n6)CC5)cc4)c3n2)c1]

Following the general procedure of Example 37, the reaction of Intermediate 6 (200 mg, 386 μmol) with 6-chloropyrazine-2-carbonitrile (59.2 mg, 424 μmol) was carried out. After purified by prep-HPLC (column: Phenomenex C18 150*25 mm*10 μm; mobile phase: [water (NH₄HCO₃)-ACN]; B %: 35%-65%, 8 min), N-(3-(2-(2-aminopyridin-3-yl)-3-(4-((4-(6-cyanopyrazin-2-yl)piperazin-1-yl)methyl)phenyl)-3H-imidazo[4,5-b]pyridin-5-yl)phenyl)acetamide (Example 40, 61.3 mg, yield: 26%) was obtained as a brown solid. MS: m/z=622.2 [M+H]⁺. ¹H NMR (400 MHz, Dimethylsulfoxide-d₆) δ 10.04 (s, 1H), 8.65 (s, 1H), 8.31-8.25 (m, 2H), 8.19 (s, 1H), 8.00 (dd, J=4.8 Hz, 1H), 7.87 (d, J=8.4 Hz, 1H), 7.70-7.61 (m, 2H), 7.54-7.44 (m, 4H), 7.38 (dd, J=8.0, 8.0 Hz, 1H), 7.15 (dd, J=7.6, 2.0 Hz, 1H), 7.00 (br s, 2H), 6.37 (dd, J=7.6, 4.8 Hz, 1H), 3.70-3.65 (m, 4H), 3.64 (s, 2H), 2.55-2.52 (m, 4H), 2.06 (s, 3H).

### Example 41: N-(3-(2-(2-Aminopyridin-3-yl)-3-(4-((4-(6-cyanopyrimidin-4-yl)piperazin-1-yl)methyl)phenyl)-3H-imidazo[4,5-b]pyridin-5-yl)phenyl)acetamide

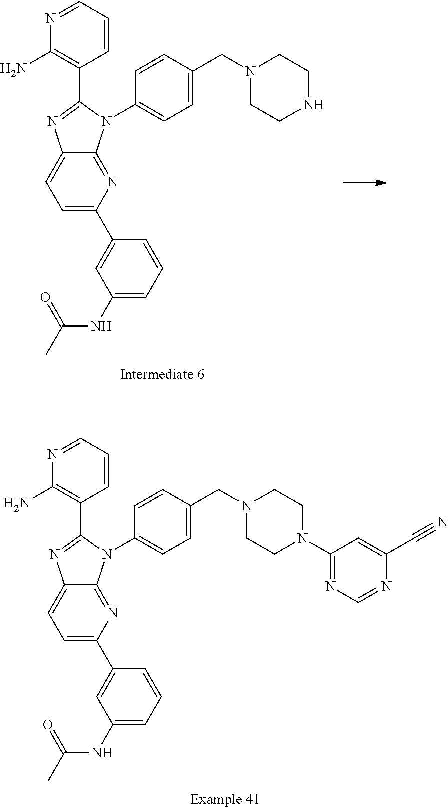 [SMILES: CC(=O)Nc1cccc(-c2ccc3nc(-c4cccnc4N)n(-c4ccc(CN5CCNCC5)cc4)c3n2)c1 ; CC(=O)Nc1cccc(-c2ccc3nc(-c4cccnc4N)n(-c4ccc(CN5CCN(c6cc(C#N)ncn6)CC5)cc4)c3n2)c1]

Following the general procedure of Example 37, the reaction of Intermediate 6 (200 mg, 386 μmol) with 6-chloropyrimidine-4-carbonitrile (59.2 mg, 424 μmol) was carried out. After purified by prep-HPLC (column: Phenomenex C18 150*25 mm*10 μm; mobile phase: [water (NH₄HCO₃)-ACN]; B %: 32%-62%, 8 min), N-(3-(2-(2-aminopyridin-3-yl)-3-(4-((4-(6-cyanopyrimidin-4-yl)piperazin-1-yl)methyl)phenyl)-3H-imidazo[4,5-b]pyridin-5-yl)phenyl)acetamide (Example 41, 59.4 mg, yield: 25%) was obtained as a white solid. MS: m/z=622.2 [M+H]⁺. ¹H NMR (400 MHz, Dimethylsulfoxide-d₆) δ 10.04 (s, 1H), 8.56 (d, J=1.2 Hz, 1H), 8.28 (d, J=8.4 Hz, 1H), 8.17 (s, 1H), 7.99 (dd, J=4.8, 2.0 Hz, 1H), 7.87 (d, J=8.4 Hz, 1H), 7.70-7.61 (m, 2H), 7.56 (s, 1H), 7.53-7.44 (m, 4H), 7.38 (dd, J=8.0, 8.0 Hz, 1H), 7.14 (dd, J=7.6, 2.0 Hz, 1H), 6.99 (br s, 2H), 6.35 (dd, J=7.6, 4.8 Hz, 1H), 3.89-3.66 (m, 4H), 3.64 (s, 2H), 2.49-2.47 (m, 4H), 2.06 (s, 3H).

### Example 42: N-(3-(2-(2-Aminopyridin-3-yl)-3-(4-((4-(2-cyanopyrimidin-5-yl)piperazin-1-yl)methyl)phenyl)-3H-imidazo[4,5-b]pyridin-5-yl)phenyl)acetamide

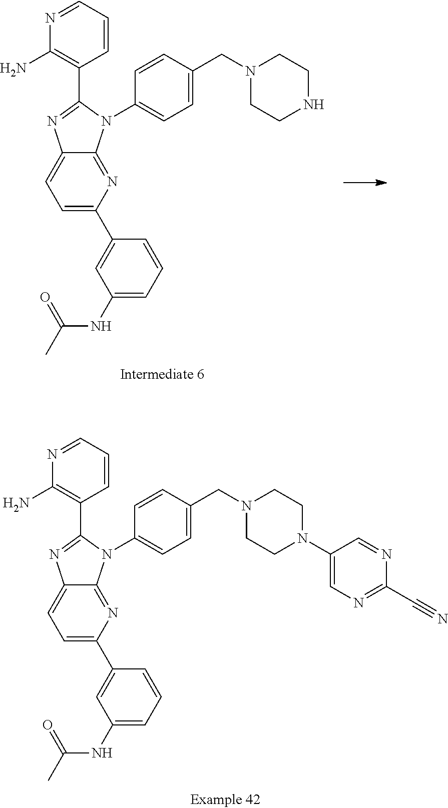 [SMILES: CC(=O)Nc1cccc(-c2ccc3nc(-c4cccnc4N)n(-c4ccc(CN5CCNCC5)cc4)c3n2)c1 ; CC(=O)Nc1cccc(-c2ccc3nc(-c4cccnc4N)n(-c4ccc(CN5CCN(c6cnc(C#N)nc6)CC5)cc4)c3n2)c1]

To a solution of Intermediate 6 (200 mg, 386 μmol) and 2-chloropyrimidine-4-carbonitrile (59 mg, 424 μmol) in 1,4-dioxane (4 mL) was added DIEA (199 mg, 1.54 mmol). The mixture was stirred at 80° C. for 12 hr. The reaction was filtered and concentrated under reduced pressure to give a residue. The crude product was purified by prep-HPLC (column: Waters xbridge 150*25 mm 10 μm; mobile phase: [water (NH₄HCO₃)-ACN]; B %: 38%-68%, 10 min), N-(3-(2-(2-aminopyridin-3-yl)-3-(4-((4-(2-cyanopyrimidin-5-yl)piperazin-1-yl)methyl)phenyl)-3H-imidazo[4,5-b]pyridin-5-yl)phenyl)acetamide (Example 42, 36.8 mg, yield: 14%) was obtained as a light-yellow solid. MS: m/z=622.3 [M+H]⁺. ¹H NMR (400 MHz, Dimethylsulfoxide-d₆) δ 10.05 (s, 1H), 8.58 (s, 2H), 8.28 (d, J=8.4 Hz, 1H), 8.17 (s, 1H), 8.00 (dd, J=4.8, 1.6 Hz, 1H), 7.86 (d, J=8.4 Hz, 1H), 7.66 (d, J=8.0 Hz, 2H), 7.57-7.45 (m, 4H), 7.38 (dd, J=8.0, 8.0 Hz, 1H), 7.14 (d, J=7.6 Hz, 1H), 6.99 (br s, 2H), 6.37 (dd, J=7.6, 4.8 Hz, 1H), 3.65 (s, 2H), 3.56-3.48 (m, 4H), 2.60-2.52 (m, 4H), 2.05 (s, 3H).

### Example 43: N-(3-(2-(2-Aminopyridin-3-yl)-3-(4-((4-(6-cyanopyridazin-4-yl)piperazin-1-yl)methyl)phenyl)-3H-imidazo[4,5-b]pyridin-5-yl)phenyl)acetamide

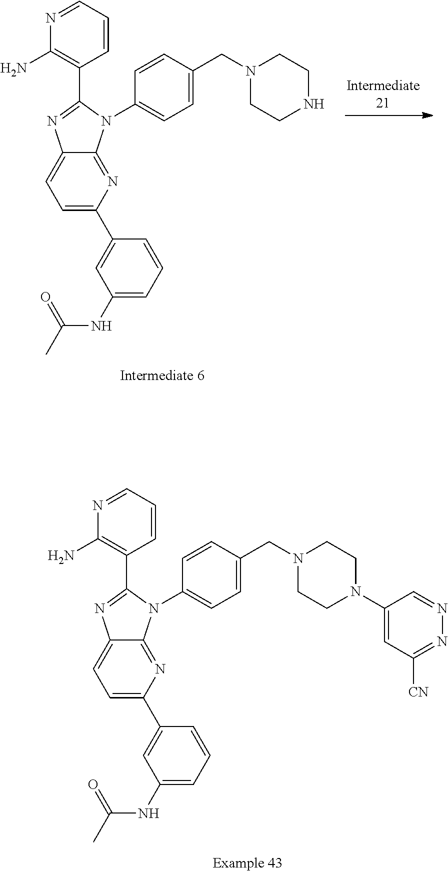 [SMILES: CC(=O)Nc1cccc(-c2ccc3nc(-c4cccnc4N)n(-c4ccc(CN5CCNCC5)cc4)c3n2)c1 ; CC(=O)Nc1cccc(-c2ccc3nc(-c4cccnc4N)n(-c4ccc(CN5CCN(c6cnnc(C#N)c6)CC5)cc4)c3n2)c1]

Following the general procedure of Example 47, the reaction of Intermediate 6 (200 mg, 385 μmol) with Intermediate 21 (54 mg, 385 μmol) was carried out. After purified by prep-HPLC (column: Waters xbridge 150*25 mm 10 μm; mobile phase: [water (NH₄HCO₃)-ACN]; B %: 25%-55%, 10 min), N-(3-(2-(2-aminopyridin-3-yl)-3-(4-((4-(6-cyanopyridazin-4-yl)piperazin-1-yl)methyl)phenyl)-3H-imidazo[4,5-b]pyridin-5-yl)phenyl)acetamide (Example 43, 54.6 mg, yield. 23%) was obtained as a yellow solid. MS: m/z=622.4 [M+H]⁺. ¹H NMR (400 MHz, Dimethylsulfoxide-d₆) δ 10.05 (s, 1H), 9.14 (d, J=3.2 Hz, 1H), 8.28 (d, J=8.4 Hz, 1H), 8.16 (s, 1H), 7.99 (dd, J=4.8, 1.6 Hz, 1H), 7.86 (d, J=8.4 Hz, 1H), 7.71-7.59 (m, 4H), 7.54-7.44 (m, 5H), 7.41-7.35 (m, 1H), 7.14 (dd, J=7.6, 1.6 Hz, 1H), 7.00 (br s, 2H), 6.35 (dd, J=8.0, 4.8 Hz, JH), 3.65 (s, 2H), 3.61-3.55 (m, 4H), 2.56-2.52 (m, 4H), 2.06 (s, 3H).

### Example 44: N-(3-(2-(2-Aminopyridin-3-yl)-3-(4-((4-(2-cyanopyrimidin-4-yl)piperazin-1-yl)methyl)phenyl)-3H-imidazo[4,5-b]pyridin-5-yl)phenyl)acetamide

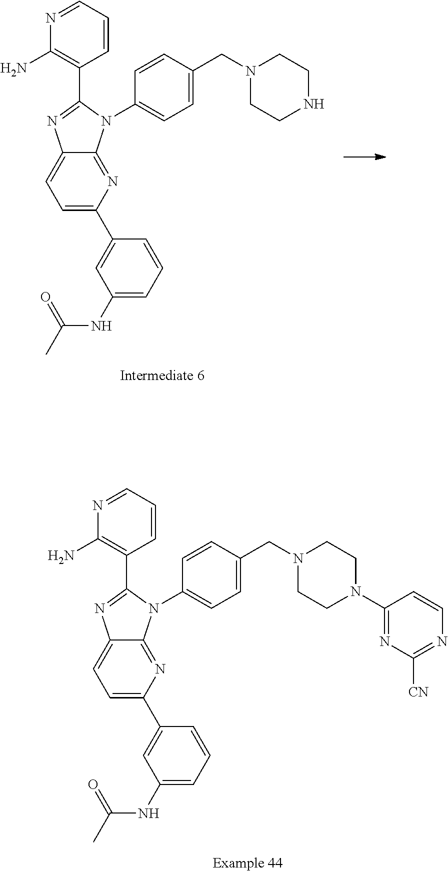 [SMILES: CC(=O)Nc1cccc(-c2ccc3nc(-c4cccnc4N)n(-c4ccc(CN5CCNCC5)cc4)c3n2)c1 ; CC(=O)Nc1cccc(-c2ccc3nc(-c4cccnc4N)n(-c4ccc(CN5CCN(c6ccnc(C#N)n6)CC5)cc4)c3n2)c1]

Following the general procedure of Example 47, the reaction of Intermediate 6 (200 mg, 385 μmol) with 4-chloropyrimidine-2-carbonitrile (54 mg, 385 μmol) was carried out. After purified by prep-HPLC (column: Waters xbridge 150*25 mm 10 μm; mobile phase: [water (NH₄HCO₃)-ACN]; B %: 32%-62%, 10 min), N-(3-(2-(2-aminopyridin-3-yl)-3-(4-((4-(2-cyanopyrimidin-4-yl)piperazin-1-yl)methyl)phenyl)-3H-imidazo[4,5-b]pyridin-5-yl)phenyl)acetamide (Example 44, 37.6 mg, yield: 16%) was obtained as a yellow solid. MS: m/z=622.5 [M+H]⁺. ¹H NMR (400 MHz, Dimethylsulfoxide-d₆) δ 10.05 (s, 1H), 8.34-8.24 (m, 2H), 8.20 (s, 1H), 8.00 (dd, J=4.8, 2.0 Hz, 1H), 7.88 (d, J=8.4 Hz, 1H), 7.70-7.62 (m, 2H), 7.57-7.45 (m, 4H), 7.42-7.36 (m, 1H), 7.18-7.10 (m, 2H), 7.00 (br s, 2H), 6.38 (dd, J=7.6, 4.8 Hz, 1H), 3.74-3.64 (m, 4H), 3.65 (s, 2H), 2.56-2.52 (m, 4H), 2.07 (s, 3H).

### Example 45: N-(3-(2-(2-Aminopyridin-3-yl)-3-(4-((4-(5-cyanopyridazin-3-yl)piperazin-1-yl)methyl)phenyl)-3H-imidazo[4,5-b]pyridin-5-yl)phenyl)acetamide

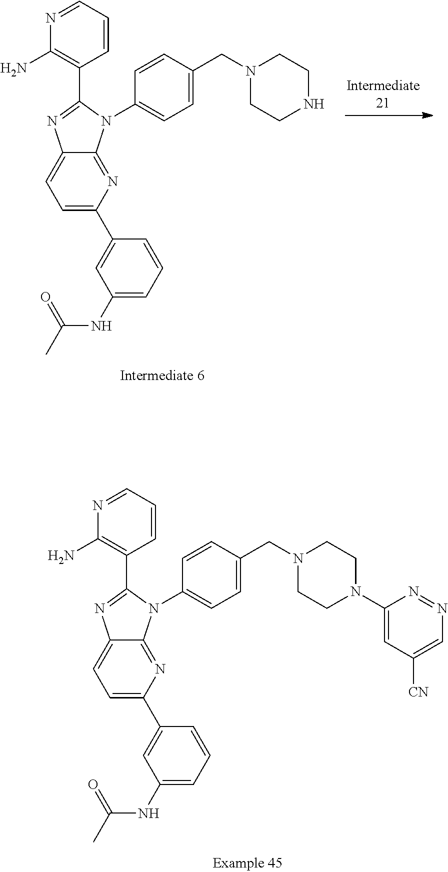 [SMILES: CC(=O)Nc1cccc(-c2ccc3nc(-c4cccnc4N)n(-c4ccc(CN5CCNCC5)cc4)c3n2)c1 ; CC(=O)Nc1cccc(-c2ccc3nc(-c4cccnc4N)n(-c4ccc(CN5CCN(c6cc(C#N)cnn6)CC5)cc4)c3n2)c1]

Following the general procedure of Example 50, the reaction of Intermediate 6 (250 mg, 482 μmol) with Intermediate 21 (74 mg, 530 μmol) was carried out. After purified by prep-HPLC (column: Waters xbridge 150*25 mm 10 μm; mobile phase: [water (NH₄HCO₃)-ACN]; B %: 25%-55%, 10 min), N-(3-(2-(2-aminopyridin-3-yl)-3-(4-((4-(5-cyanopyridazin-3-yl)piperazin-1-yl)methyl)phenyl)-3H-imidazo[4,5-b]pyridin-5-yl)phenyl)acetamide (Example 45, 6 mg, yield: 2%) was obtained as a yellow solid. MS: m/z=622.2 [M+H]⁺. ¹H NMR (400 MHz, Dimethylsulfoxide-d₆) δ 8.68 (s, 1H), 8.25-8.16 (m, 2H), 7.99 (dd, J=5.2, 1.2 Hz, 1H), 7.92 (d, J=8.4 Hz, 1H), 7.77 (d, J=8.0 Hz, 1H), 7.67 (s, 1H), 7.64-7.56 (m, 3H), 7.47 (d, J=8.4 Hz, 2H), 7.41-7.30 (m, 2H), 6.47 (dd, J=7.6, 4.8 Hz, 1H), 3.79-3.74 (m, 4H), 3.70 (s, 2H), 2.70-2.63 (m, 4H), 2.14 (s, 3H).

### Example 46: N-(3-(2-(2-Aminopyridin-3-yl)-3-(4-((4-(4-cyanopyrimidin-2-yl)piperazin-1-yl)methyl)phenyl)-3H-imidazo[4,5-b]pyridin-5-yl)phenyl)acetamide

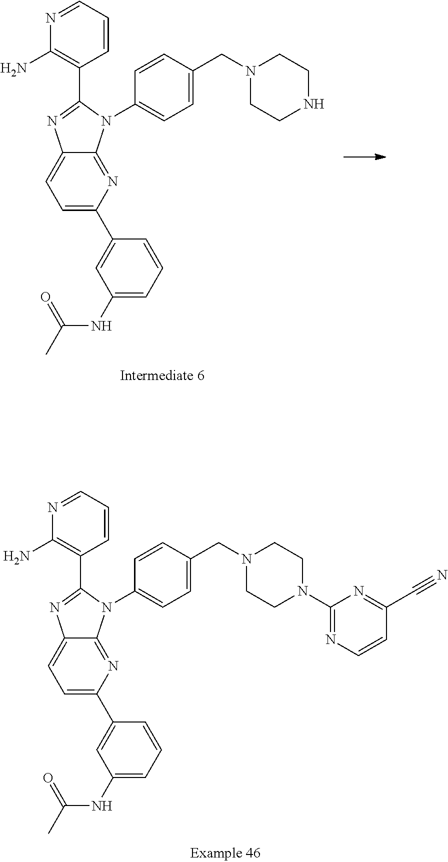 [SMILES: CC(=O)Nc1cccc(-c2ccc3nc(-c4cccnc4N)n(-c4ccc(CN5CCNCC5)cc4)c3n2)c1 ; CC(=O)Nc1cccc(-c2ccc3nc(-c4cccnc4N)n(-c4ccc(CN5CCN(c6nccc(C#N)n6)CC5)cc4)c3n2)c1]

To a solution of Intermediate 6 (200 mg, 386 μmol) and 2-chloropyrimidine-4-carbonitrile (59 mg, 424 μmol) in 1,4-dioxane (4 mL) was added DIEA (199 mg, 1.54 mmol). The mixture was stirred at 80° C. for 12 hr. The reaction was filtered and concentrated under reduced pressure to give a residue. The crude product was purified by prep-HPLC (column: Waters xbridge 150*25 mm 10 μm; mobile phase: [water (NH₄HCO₃)-ACN]; B %: 38%-68%, 10 min), N-(3-(2-(2-aminopyridin-3-yl)-3-(4-((4-(4-cyanopyrimidin-2-yl)piperazin-1-yl)methyl)phenyl)-3H-imidazo[4,5-b]pyridin-5-yl)phenyl)acetamide (Example 46, 36.8 mg, yield: 14%) was obtained as a light-yellow solid. MS: m/z=622.3 [M+H]⁺. ¹H NMR (400 MHz, Dimethylsulfoxide-d₆) δ 10.04 (s, 1H), 8.64 (d, J=4.4 Hz, 1H), 8.28 (d, J=8.4 Hz, 1H), 8.19 (s, 1H), 7.99 (dd, J=4.8, 2.0 Hz, 1H), 7.87 (d, J=8.4 Hz, 1H), 7.70-7.60 (m, 2H), 7.57-7.43 (m, 4H), 7.40-7.34 (m, 1H), 7.24-7.09 (m, 2H), 6.99 (br s, 2H), 6.37 (dd, J=7.6, 4.8 Hz, 1H), 3.83-3.73 (m, 4H), 3.63 (s, 2H), 3.33-3.31 (m, 4H), 2.06 (s, 3H).

### Example 47: 4-((4-(2-(2-Aminopyridin-3-yl)-3H-imidazo[4,5-b]pyridin-3-yl)benzyl)amino)pyrimidine-2-carbonitrile

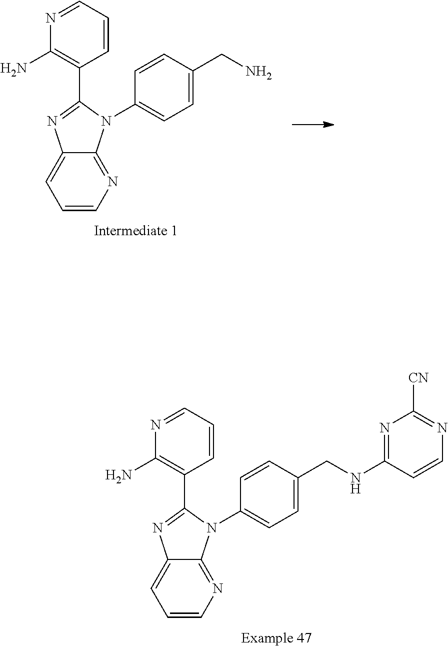 [SMILES: NCc1ccc(-n2c(-c3cccnc3N)nc3cccnc32)cc1 ; N#Cc1nccc(NCc2ccc(-n3c(-c4cccnc4N)nc4cccnc43)cc2)n1]

A mixture of Intermediate 1 (200 mg, 632 μmol), 4-chloropyrimidine-2-carbonitrile (88 mg, 632 μmol) and DIEA (408 mg, 3.16 mmol) in NMP (3 mL) was stirred at 130° C. for 1 hr under microwave. The reaction mixture was poured into H₂O (50 mL). The resulting mixture was extracted with CH₂Cl₂ (50 mL×3). The combined organic dried over anhydrous Na₂SO₄, filtered and concentrated to dryness. The residue was purified by prep-HPLC (column: Waters xbridge 150*25 mm 10 μm; mobile phase: [water (NH₄HCO₃)-ACN]; B %: 20%-50%, 10 min), 4-((4-(2-(2-aminopyridin-3-yl)-3H-imidazo[4,5-b]pyridin-3-yl)benzyl)amino)pyrimidine-2-carbonitrile (Example 47, 26.3 mg, yield: 10%) as a light-yellow lyophilized powder. MS: m/z=420.3 [M+H]⁺. ¹H NMR (400 MHz, Dimethylsulfoxide-d₆) δ 8.70-8.56 (m, 1H), 8.31 (d, J=4.8 Hz, 1H), 8.26-8.13 (m, 2H), 7.99 (dd, J=4.8, 1.6 Hz, 1H), 7.47 (d, J=8.4 Hz, 2H), 7.45-7.37 (m, 3H), 7.22 (d, J=7.6 Hz, 1H), 6.96 (br s, 2H), 6.81 (d, J=6.0 Hz, 1H), 6.40 (dd, J=6.8, 4.8 Hz, 1H), 4.63 (d, J=5.2 Hz, 2H).

### Example 48: 4-((4-(2-(2-Aminopyridin-3-yl)-5-phenyl-3H-imidazo[4,5-b]pyridin-3-yl)benzyl)amino)pyrimidine-2-carbonitrile

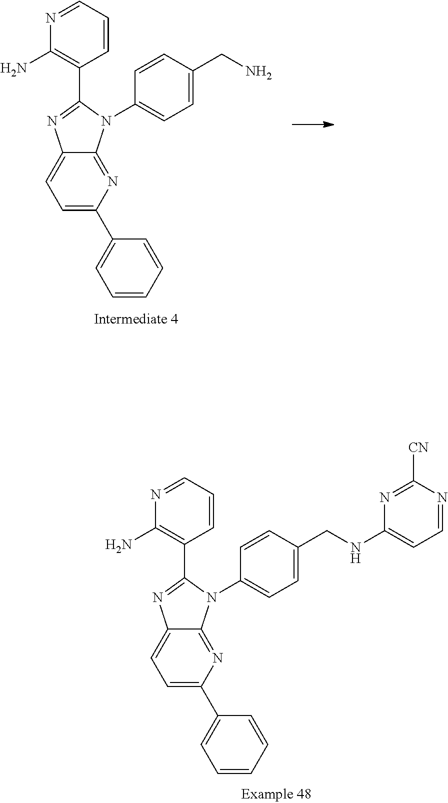 [SMILES: NCc1ccc(-n2c(-c3cccnc3N)nc3ccc(-c4ccccc4)nc32)cc1 ; N#Cc1nccc(NCc2ccc(-n3c(-c4cccnc4N)nc4ccc(-c5ccccc5)nc43)cc2)n1]

Following the general procedure of Example 47, the reaction of Intermediate 4 (200 mg, 509 μmol) with 4-chloropyrimidine-2-carbonitrile (71 mg, 509 μmol) was carried out. After purified by prep-HPLC (column: Waters xbridge 150*25 mm 10 μm; mobile phase: [water (NH₄HCO₃)-ACN]; B %: 40%-70%, 10 min), 4-((4-(2-(2-aminopyridin-3-yl)-5-phenyl-3H-imidazo[4,5-b]pyridin-3-yl)benzyl)amino)pyrimidine-2-carbonitrile (Example 48, 36.4 mg, yield: 14.4%) was obtained as a yellow solid. MS: m/z=496.4 [M+H]⁺. ¹H NMR (400 MHz, Dimethylsulfoxide-d₆) δ 64 (t, J=5.6 Hz, 1H), 8.27 (d, J=8.4 Hz, 1H), 8.17 (d, J=5.6 Hz, 1H), 8.05-7.97 (m, 4H), 7.52-7.44 (m, 6H), 7.42-7.36 (m, 1H), 7.24-7.15 (m, 1H), 6.96 (br s, 2H), 6.82 (d, J=6.0 Hz, 1H), 6.44-6.36 (m, 1H), 4.66 (J=5.6 Hz, 2H).

### Example 49: 4-(4-(4-(2-(2-Aminopyridin-3-yl)-5-phenyl-3H-imidazo[4,5-b]pyridin-3-yl)benzyl)piperazin-1-yl)pyrimidine-2-carbonitrile

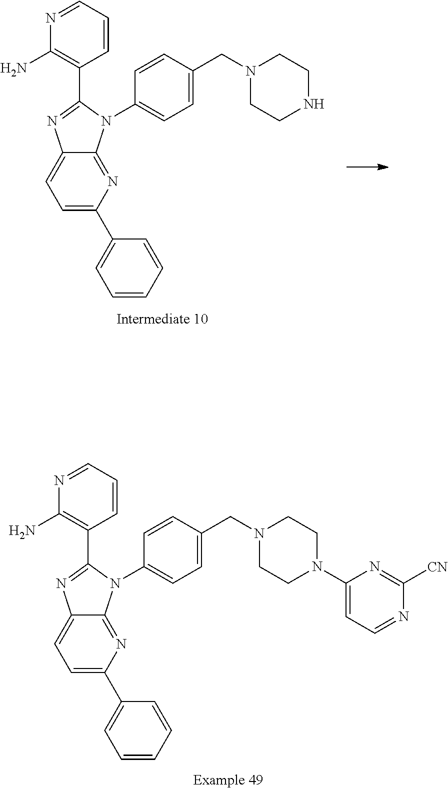 [SMILES: Nc1ncccc1-c1nc2ccc(-c3ccccc3)nc2n1-c1ccc(CN2CCNCC2)cc1 ; N#Cc1nccc(N2CCN(Cc3ccc(-n4c(-c5cccnc5N)nc5ccc(-c6ccccc6)nc54)cc3)CC2)n1]

Following the general procedure of Example 47, the reaction of Intermediate 10 (200 mg, 433 μmol) with 4-chloropyrimidine-2-carbonitrile (60 mg, 433 μmol) was carried out. After purified by prep-HPLC (column; Waters xbridge 150*25 mm 10 μm; mobile phase: [water (NH₄HCO₃)-ACN]; B %: 47%-77%, 10 min), 4-(4-(4-(2-(2-aminopyridin-3-yl)-5-phenyl-3H-imidazo[4,5-b]pyridin-3-yl)benzyl)piperazin-1-yl)pyrimidine-2-carbonitrile (Example 49, 73.9 mg, yield: 30%) was obtained as a yellow solid. MS: m/z=565.4 [M+H]⁺. ¹H NMR (400 MHz, Dimethylsulfoxide-d₆) δ 8.32-8.22 (m, 2H), 8.07-7.95 (m, 4H), 7.54-7.44 (m, 6H), 7.43-7.37 (m, 1H), 7.18-7.08 (m, 2H), 7.03 (br s, 2H), 6.37 (dd, J=7.6, 4.8 Hz, 1H), 3.72-3.66 (m, 4H), 3.65 (s, 2H), 2.57-2.51 (m, 4H).

### Example 50: 6-((4-(2-(2-Aminopyridin-3-yl)-3H-imidazo[4,5-b]pyridin-3-yl)benzyl)amino)pyridazine-4-carbonitrile

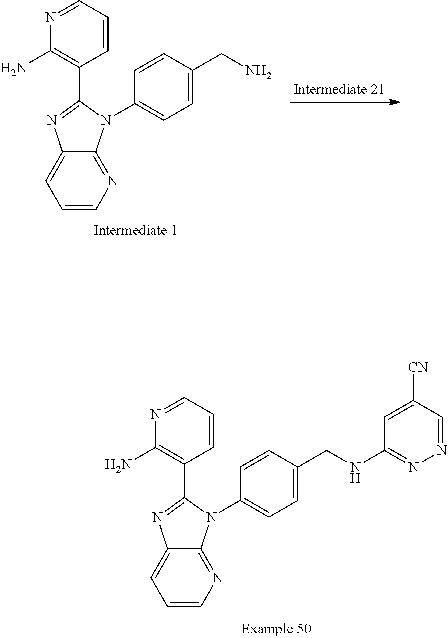 [SMILES: NCc1ccc(-n2c(-c3cccnc3N)nc3cccnc32)cc1 ; N#Cc1cnnc(NCc2ccc(-n3c(-c4cccnc4N)nc4cccnc43)cc2)c1]

To a solution of Intermediate 1 in NMP (2 mL) was added Intermediate 21 (106 mg, 759 μmol) and DIEA (408 mg, 3.16 mmol). The mixture was stirred at microwave 160° C. for 1 hr. The mixture was concentrated under reduced pressure to give a residue. The mixture was purified by prep-HPLC (column: Welch Xtimate C18 150*25 mm*5 μm; mobile phase: [water (HCl)-ACN]; B %: 18%-48%, 10 min) to give 6-((4-(2-(2-aminopyridin-3-yl)-3H-imidazo[4,5-b]pyridin-3-yl)benzyl)amino)pyridazine-4-carbonitrile (Example 50, 11.3 mg, yield; 4.3%) was a light-gray solid. MS: m/z=420.1 [M+H]⁺. 1H NMR (400 MHz, Dimethylsulfoxide-d₆) δ 8.75 (d, J=1.6 Hz, 1H), 8.30 (dd, J=4.8, 1.2 Hz, 1H), 8.19 (dd, J=8.0, 1.2 Hz, 1H), 8.08-7.92 (m, 2H), 7.49 (d, J=8.4 Hz, 2H), 7.44-7.29 (m, 4H), 7.21 (dd, J=7.6, 1.6 Hz, 1H), 6.96 (br s, 2H), 6.39 (dd, J=7.6, 4.8 Hz, 1H), 4.70 (d, J=5.6 Hz, 2H).

### Example 51: 6-((1-(4-(2-(2-Aminopyridin-3-yl)-3H-imidazo[4,5-b]pyridin-3-yl)benzyl)piperidin-4-yl)amino)pyridazine-4-carbonitrile

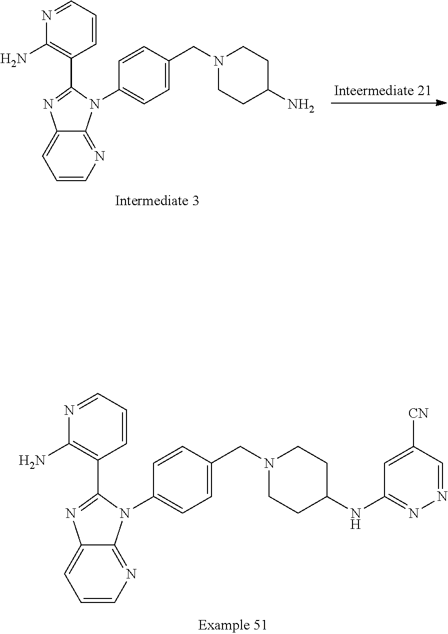 [SMILES: Nc1ncccc1-c1nc2cccnc2n1-c1ccc(CN2CCC(N)CC2)cc1 ; N#Cc1cnnc(NC2CCN(Cc3ccc(-n4c(-c5cccnc5N)nc5cccnc54)cc3)CC2)c1]

Following the general procedure of Example 50, the reaction of Intermediate 3 (20 mg, 50 μmol) with Intermediate 21 (8.4 mg, 60 μmol) was carried out. After purified by prep-HPLC (column: Phenomenex C18 150*25 mm*10 μm; mobile phase: [water (NH₄HCO₃)-ACN]; B %: 25%-55%, 8 min), 6-((1-(4-(2-(2-aminopyridin-3-yl)-3H-imidazo[4,5-b]pyridin-3-yl)benzyl)piperidin-4-yl)amino)pyridazine-4-carbonitrile (Example 51, 9 mg, yield: 36%) was obtained as a light-gray solid. MS: m/z=503.2 [M+H]⁺. ¹H NMR (400 MHz, Dimethylsulfoxide-d₆) δ 8.68 (d, J=1.6 Hz, 1H), 8.33 (dd, J=4.8, 1.2 Hz, 1H), 8.20 (dd, J=8.0, 1.2 Hz, 1H), 7.98 (dd, J=4.8, 1.6 Hz, 1H), 7.46 (d, J=8.4 Hz, 2H), 7.42-7.32 (m, 4H), 7.22 (s, 1H), 7.17 (dd, J=7.6, 1.6 Hz, 1H), 7.00 (br s, 2H), 6.37 (dd, J=7.6, 4.8 Hz, 1H), 3.96-3.76 (m, 1H), 3.57 (s, 2H), 2.83 (d, J=11.2 Hz, 2H), 2.14 (t, J=11.2 Hz, 2H), 1.96 (d, J=10.0 Hz, 2H), 1.58-1.44 (m, 2H).

### Example 52: 6-((4-(2-(2-Aminopyridin-3-yl)-5-phenyl-3H-imidazo[4,5-b]pyridin-3-yl)benzyl)amino)pyridazine-4-carbonitrile

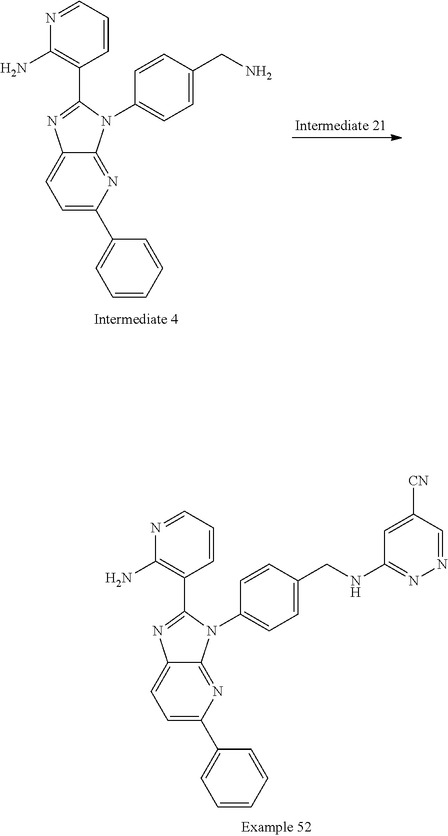 [SMILES: NCc1ccc(-n2c(-c3cccnc3N)nc3ccc(-c4ccccc4)nc32)cc1 ; N#Cc1cnnc(NCc2ccc(-n3c(-c4cccnc4N)nc4ccc(-c5ccccc5)nc43)cc2)c1]

Following the general procedure of Example 50, the reaction of Intermediate 4 (20 mg, 50 μmol) with Intermediate 21 (8.5 mg, 61 μmol) was carried out. After purified by prep-HPLC (column: Phenomenex C18 150*25 mm*10 μm; mobile phase: [water (NH₄HCO₃)-ACN]; B %: 30%-60%, 8 min), 6-((4-(2-(2-aminopyridin-3-yl)-5-phenyl-3H-imidazo[4,5-b]pyridin-3-yl)benzyl)amino)pyridazine-4-carbonitrile (Example 52, 9 mg, yield: 34%) was obtained as a light-gray solid. MS: m/z=496.1 [M+H]⁺. ¹H NMR (400 MHz, Dimethylsulfoxide-d₆) δ 8.77 (d, J=1.6 Hz, 1H), 8.26 (d, J=8.4 Hz, 1H), 8.05-7.95 (m, 5H), 7.56-7.50 (m, 2H), 7.50-7.44 (m, 4H), 7.41-7.38 (m, 1H), 7.35 (d, J=1.6 Hz, 1H), 7.20 (dd, J=7.6, 1.6 Hz, 1H), 6.95 (br s, 2H), 6.40 (dd, J=7.6, 4.8 Hz, 1H), 4.74 (d, J=5.6 Hz, 2H).

### Example 53: 6-(4-(4-(2-(2-Aminopyridin-3-yl)-5-phenyl-3H-imidazo[4,5-b]pyridin-3-yl)benzyl)piperazin-1-yl)pyridazine-4-carbonitrile

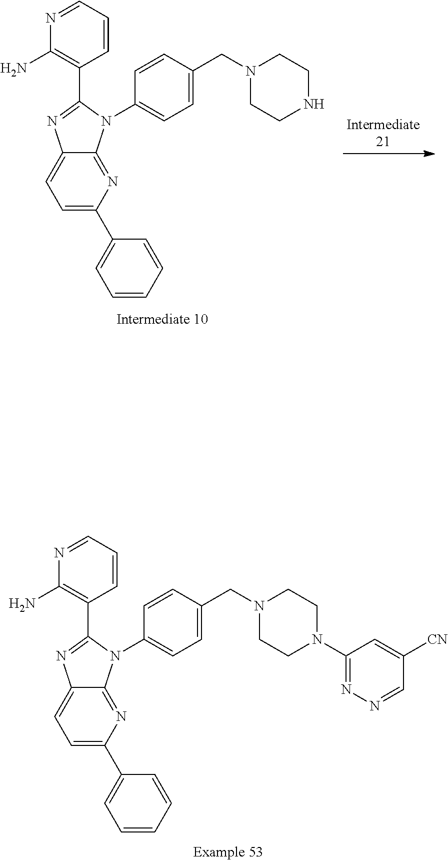 [SMILES: Nc1ncccc1-c1nc2ccc(-c3ccccc3)nc2n1-c1ccc(CN2CCNCC2)cc1 ; N#Cc1cnnc(N2CCN(Cc3ccc(-n4c(-c5cccnc5N)nc5ccc(-c6ccccc6)nc54)cc3)CC2)c1]

Following the general procedure of Example 50, the reaction of Intermediate 10 (200 mg, 433 μmol) with Intermediate 21 (73 mg, 520 μmol) was carried out. After purified by prep-HPLC (column: Waters xbridge 150*25 mm 10 μm; mobile phase: [water (NH₄HCO₃)-ACN]; B %: 42%-72%, 10 min) to 6-(4-(4-(2-(2-aminopyridin-3-yl)-5-phenyl-3H-imidazo[4,5-b]pyridin-3-yl)benzyl)piperazin-1-yl)pyridazine-4-carbonitrile (Example 53, 15.2 mg, yield: 6.2%) was obtained as a light-gray solid. MS: m/z=565.2 [M+H]⁺. ¹H NMR (400 MHz, Dimethylsulfoxide-d₆) δ 8.84 (d, J=1.2 Hz, 1H), 8.27 (d, J=8.4 Hz, 1H), 8.16-7.96 (m, 4H), 7.87 (d, J=1.2 Hz, 1H), 7.56-7.51 (m, 2H), 7.51-7.44 (m, 4H), 7.43-7.37 (m, 1H), 7.16 (dd, J=7.6, 1.6 Hz, 1H), 7.04 (br s, 2H), 6.37 (dd, J=7.6, 4.8 Hz, 1H), 3.75-3.68 (m, 4H), 3.65 (s, 2H), 2.56-2.53 (m, 4H).

### Example 54: 6-((1-(4-(2-(2-Aminopyridin-3-yl)-5-phenyl-3H-imidazo[4,5-b]pyridin-3-yl)benzyl)piperidin-4-yl)amino)pyridazine-4-carbonitrile

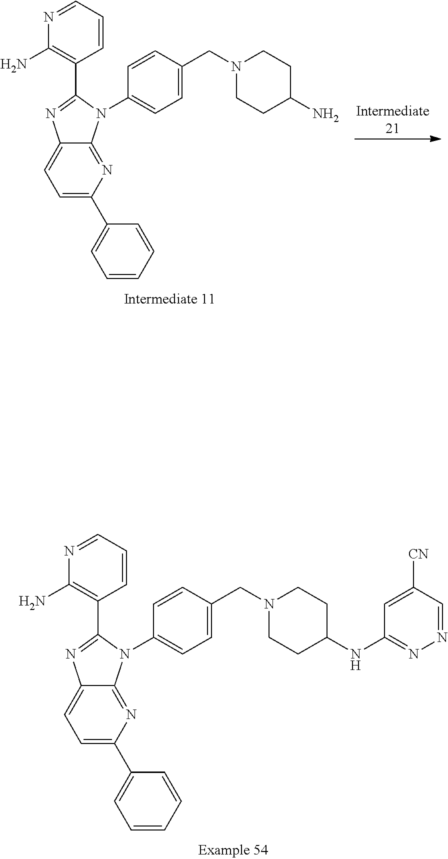 [SMILES: Nc1ncccc1-c1nc2ccc(-c3ccccc3)nc2n1-c1ccc(CN2CCC(N)CC2)cc1 ; N#Cc1cnnc(NC2CCN(Cc3ccc(-n4c(-c5cccnc5N)nc5ccc(-c6ccccc6)nc54)cc3)CC2)c1]

Following the general procedure of Example 50, the reaction of Intermediate 11 (20 mg, 42 μmol) with Intermediate 21 (7 mg, 50 μmol) was carried out. After purified by prep-HPLC (column: Phenomenex C18 150*25 mm*10 μm; mobile phase: [water (NH₄HCO₃)-ACN], B %: 35%-65%, 8 min), 6-((1-(4-(2-(2-aminopyridin-3-yl)-5-phenyl-3H-imidazo[4,5-b]pyridin-3-yl)benzyl)piperidin-4-yl)amino)pyridazine-4-carbonitrile (Example 54, 10 mg, yield: 41%) was obtained as a light-gray solid. MS: m/z=579.3 [M+H]⁺. ¹H NMR (400 MHz, Dimethylsulfoxide-d₆) δ 8.68 (d, J=1.6 Hz, 1H), 8.27 (d, J=8.4 Hz, 1H), 8.09-7.93 (m, 4H), 7.57-7.42 (m, 6H), 7.41-7.36 (m, 2H), 7.22 (s, 1H), 7.16 (dd, J=7.6, 1.6 Hz, 1H), 7.03 (br s, 2H), 6.37 (dd, J=7.6, 4.8 Hz, 1H), 3.95-3.75 (m, 1H), 3.59 (s, 2H), 2.84 (d, J=11.2 Hz, 2H), 2.16 (t, J=11.2 Hz, 2H), 1.97 (d, J=11.6 Hz, 2H), 1.60-1.43 (m, 2H).

### Example 55: N-(1-(4-(2-(2-Aminopyridin-3-yl)-5-phenyl-3H-imidazo[4,5-b]pyridin-3-yl)benzyl)piperidin-4-yl)-2-cyanoisonicotinamide

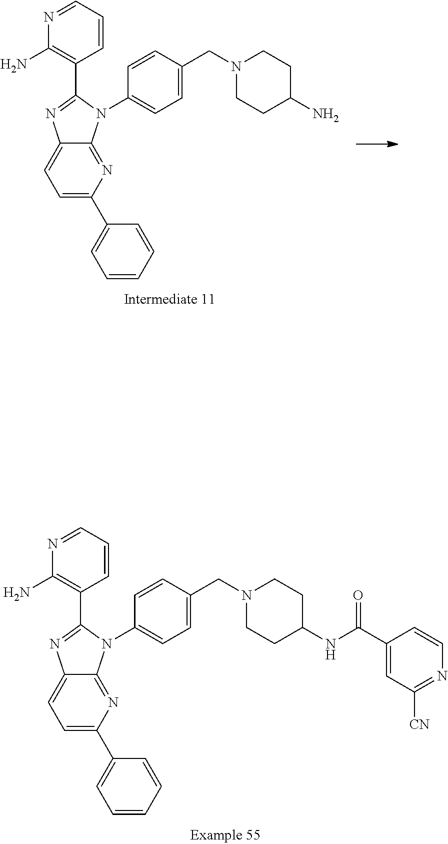 [SMILES: Nc1ncccc1-c1nc2ccc(-c3ccccc3)nc2n1-c1ccc(CN2CCC(N)CC2)cc1 ; N#Cc1cc(C(=O)NC2CCN(Cc3ccc(-n4c(-c5cccnc5N)nc5ccc(-c6ccccc6)nc54)cc3)CC2)ccn1]

Following the general procedure of Example 23, the reaction of Intermediate 11 (165 mg, 347 μmol) with 2-cyanopyridine-4-carboxylic acid ((61.7 mg, 416 pnol) was carried out. After purified by prep-HPLC (column: Waters xbridge 150*25 mm 10 μm, mobile phase: [water (NH₄HCO₃)-ACN]; B %: 38%-68%, 8 min), N-(1-(4-(2-(2-aminopyridin-3-yl)-5-phenyl-3H-imidazo[4,5-b]pyridin-3-yl)benzyl)piperidin-4-yl)-2-cyanoisonicotinamide (Example 55, 102 mg, yield: 48%) was obtained as a off-white solid. MS: m/z=606.2 [M+H]⁺. ¹H NMR (400 MHz, Chloroform-d) δ 8.84 (d, J=4.8 Hz, 1H), 8.13 (d, J=8.4 Hz, 1H), 8.07 (dd, J=5.2, 1.6 Hz, 1H), 8.04-8.00 (m, 3H), 7.84-7.79 (m, 2H), 7.50 (d, J=8.4 Hz, 2H), 7.45-7.37 (m, 5H), 7.09 (dd, J=8.0, 1.6 Hz, 1H), 6.59 (br s, 2H), 6.36 (dd, J=7.6, 4.8 Hz, 1H), 6.16 (br s, 1H), 4.14-4.01 (m, 1H), 3.65 (s, 2H), 2.97 (d, J=11.6 Hz, 2H), 2.31-2.24 (m, 2H), 2.11-2.05 (m, 2H), 1.75-1.68 (m, 2H).

### Example 56: N-(1-(4-(2-(2-Aminopyridin-3-yl)-5-phenyl-3H-imidazo[4,5-b]pyridin-3-yl)benzyl)piperidin-4-yl)-6-cyanopicolinamide

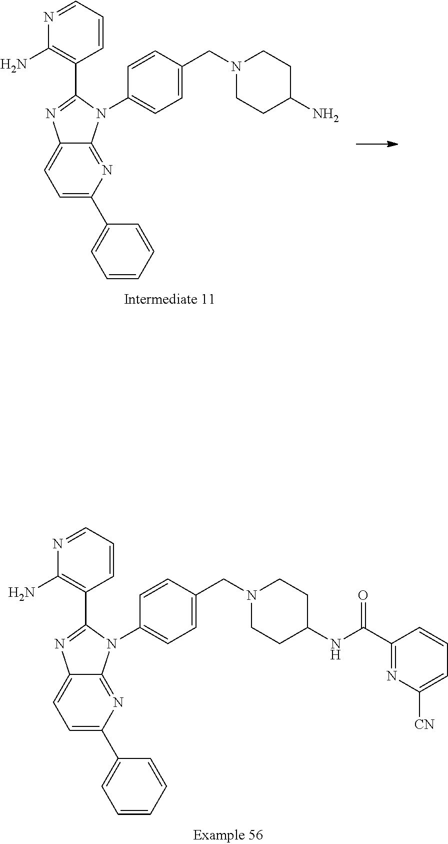 [SMILES: Nc1ncccc1-c1nc2ccc(-c3ccccc3)nc2n1-c1ccc(CN2CCC(N)CC2)cc1 ; N#Cc1cccc(C(=O)NC2CCN(Cc3ccc(-n4c(-c5cccnc5N)nc5ccc(-c6ccccc6)nc54)cc3)CC2)n1]

Following the general procedure of Example 23, the reaction of Intermediate 11 (250 mg, 526 μmol) with 6-cyanopyridine-2-carboxylic acid (93 mg, 631 μmol) was carried out. After purified by prep-HPLC (column: Phenomenex C18 150*25 mm*10 μm; mobile phase: [water (NH₄HCO₃)-ACN]; B %: 33%-63%, 8 min), N-(1-(4-(2-(2-aminopyridin-3-yl)-5-phenyl-3H-imidazo[4,5-b]pyridin-3-yl)benzyl)piperidin-4-yl)-6-cyanopicolinamide (Example 56, 57.3 mg, yield: 18%) was obtained as a yellow solid. MS: m/z=606.2 [M+H]⁺. ¹H NMR (400 MHz, Chloroform-d) δ 8.42 (d, J=8.0 Hz, 1H), 8.13 (d, J=8.4 Hz, 1H), 8.06 (dd, J=4.8, 1.6 Hz, 1H), 8.05-7.99 (m, 3H), 7.85-7.79 (m, 3H), 7.57 (d, J=8.0 Hz, 2H), 7.47-7.41 (m, 4H), 7.40-7.34 (m, 1H), 7.11 (dd, J=7.6, 1.2 Hz, 1H), 6.65 (br s, 2H), 6.37 (dd, J=7.6, 4.8 Hz, 1H), 4.13-4.01 (m, 1H), 3.73 (s, 2H), 310-2.96 (m, 2H), 2.45-2.31 (m, 2H), 2.10 (d, J=10.8 Hz, 2H), 1.87-1.80 (m, 2H).

### Example 57: N-(1-(4-(2-(2-Aminopyridin-3-yl)-5-phenyl-3H-imidazo[4,5-b]pyridin-3-yl)benzyl)piperidin-4-yl)-5-cyanonicotinamide

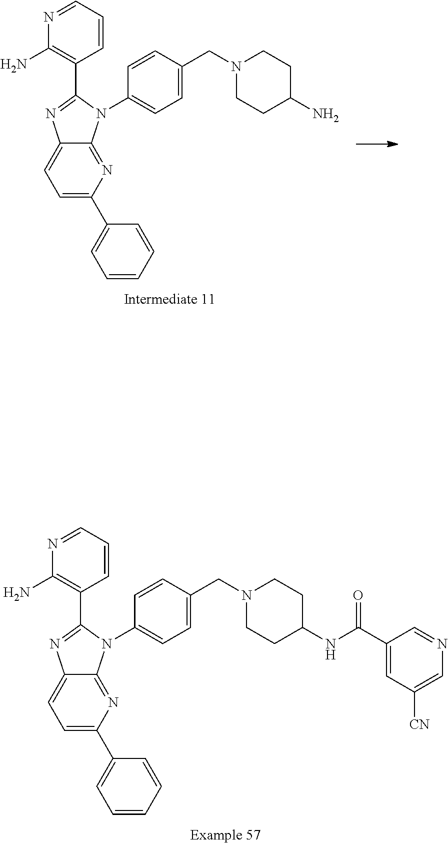 [SMILES: Nc1ncccc1-c1nc2ccc(-c3ccccc3)nc2n1-c1ccc(CN2CCC(N)CC2)cc1 ; N#Cc1cncc(C(=O)NC2CCN(Cc3ccc(-n4c(-c5cccnc5N)nc5ccc(-c6ccccc6)nc54)cc3)CC2)c1]

Following the general procedure of Example 23, the reaction of Intermediate 11 (250 mg, 526 μmol) with and 5-cyanopyridine-3-carboxylic acid (93 mg, 631 μmol) was carried out. After purified by prep-HPLC (column: Phenomenex C18 150*25 mm*10 μm; mobile phase: [water (NH₄HCO₃)-ACN]; B %: 40%-70%, 8 min), N-(1-(4-(2-(2-aminopyridin-3-yl)-5-phenyl-3H-imidazo[4,5-b]pyridin-3-yl)benzyl)piperidin-4-yl)-5-cyanonicotinamide (Example 57, 80.9 mg, yield: 25%) was obtained as a yellow solid. MS: m/z=606.2 [M+H]⁺. ¹H NMR (400 MHz, Chloroform-d) δ 9.16 (d, J=2.0 Hz, 1H), 8.96 (d, J=2.0 Hz, 1H), 8.48-8.40 (m, 1H), 8.13 (d, J=8.4 Hz, 1H), 8.06 (dd, J=4.8, 1.6 Hz, 1H), 8.03-7.99 (m, 2H), 7.80 (d, J=8.4 Hz, 1H), 7.52 (d, J=8.4 Hz, 2H), 7.45-7.40 (m, 4H), 7.39-7.35 (m, 1H), 7.08 (dd, J=7.6, 1.6 Hz, 1H), 6.59 (br s, 2H), 6.43 (br s, 1H), 6.35 (dd, J=7.6, 4.8 Hz, 1H), 4.15-4.05 (m, 1H), 3.70 (s, 2H), 3.02 (d, J=11.2 Hz, 2H), 2.33 (t, 1=11.2 Hz, 2H), 2.10 (d, J=10.0 Hz, 2H), 1.81-1.71 (m, 2H).

### Example 58: N-(1-(4-(2-(2-Aminopyridin-3-yl)-5-phenyl-3H-imidazo[4,5-b]pyridin-3-yl)benzyl)piperidin-4-yl)-2-cyanonicotinamide

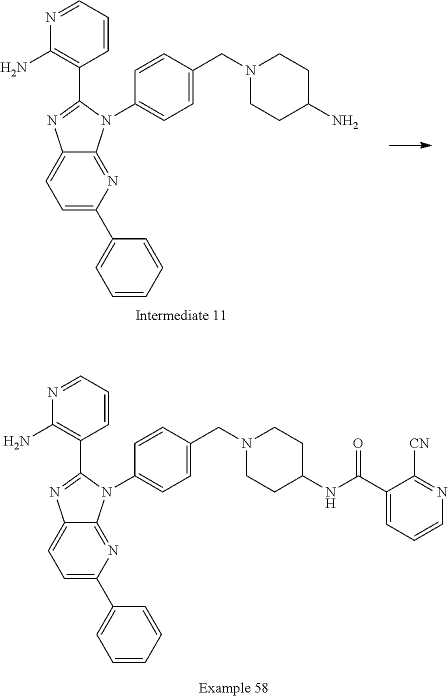 [SMILES: Nc1ncccc1-c1nc2ccc(-c3ccccc3)nc2n1-c1ccc(CN2CCC(N)CC2)cc1 ; N#Cc1ncccc1C(=O)NC1CCN(Cc2ccc(-n3c(-c4cccnc4N)nc4ccc(-c5ccccc5)nc43)cc2)CC1]

Following the general procedure of Example 23, the reaction of Intermediate 11 (200 mg, 421 μmol) with 2-cyanopyridine-3-carboxylic acid (75 mg, 505 μmol) was carried out. After purified by prep-HPLC (column: Waters xbridge 150*25 mm 10 μm; mobile phase: [water (NH₄HCO₃)-ACN]; B %: 34%-64%, 8 min), N-(1-(4-(2-(2-aminopyridin-3-yl)-5-phenyl-3H-imidazo[4,5-b]pyridin-3-yl)benzyl)piperidin-4-yl)-2-cyanonicotinamide (Example 58, 55.9 mg, yield: 22%) was obtained as a yellow solid. MS: m/z=606.4 [M+H]⁺. ¹H NMR (400 MHz, Dimethylsulfoxide-d₆) δ 9.89 (s, 1H), 8.92 (d, J=4.4 Hz, 1H), 8.33-8.19 (m, 2H), 8.03 (d, J=7.6 Hz, 2H), 8.01-7.93 (m, 2H), 7.73 (dd, J=7.6, 5.2 Hz, 1H), 7.57-7.50 (m, 2H), 7.50-7.42 (m, 4H), 7.41-7.34 (m, 1H), 7.17 (d, J=6.8 Hz, 1H), 7.04 (br s, 2H), 6.38 (dd, J=7.6, 4.8 Hz, 1H), 4.38-4.25 (m, 1H), 3.62 (s, 2H), 2.98 (d, J=11.2 Hz, 2H), 2.64-2.55 (m, 2H), 2.16-2.04 (m, 2H), 1.67 (d, J=10.0 Hz, 2H).

### Example 59: N-(1-(4-(2-(2-Aminopyridin-3-yl)-5-phenyl-3H-imidazo[4,5-b]pyridin-3-yl)benzyl)piperidin-4-yl)-3-cyanoisonicotinamide

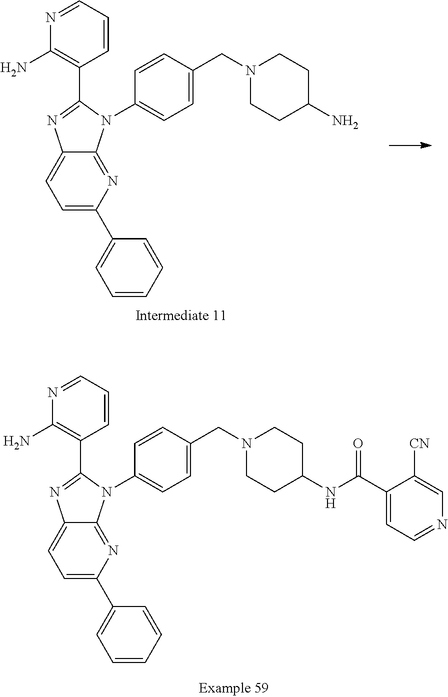 [SMILES: Nc1ncccc1-c1nc2ccc(-c3ccccc3)nc2n1-c1ccc(CN2CCC(N)CC2)cc1 ; N#Cc1cnccc1C(=O)NC1CCN(Cc2ccc(-n3c(-c4cccnc4N)nc4ccc(-c5ccccc5)nc43)cc2)CC1]

Following the general procedure of Example 23, the reaction of Intermediate 1 (200 mg, 421 μmol) with 3-cyanopyridine-4-carboxylic acid (75 mg, 505 μmol) was carried out. After purified by prep-HPLC (column: Waters xbridge 150*25 mm 10 μm; mobile phase: [water (NH₄HCO₃)-ACN]; B %: 33%-63%, 8 min), N-(1-(4-(2-(2-aminopyridin-3-yl)-5-phenyl-3H-imidazo[4,5-b]pyridin-3-yl)benzyl)piperidin-4-yl)-3-cyanoisonicotinamide (Example 59, 67.8 mg, yield: 27%) was obtained as a yellow solid. MS: m/z=606.2 [M+H]⁺. ¹H NMR (400 MHz, Dimethylsulfoxide-d₆) δ 10.54 (s, 1H), 9.40 (s, 1H), 8.94 (d, J=4.8 Hz, 1H), 8.27 (d, J=8.4 Hz, 1H), 8.04 (d, J=7.6 Hz, 2H), 8.01-7.96 (m, 2H), 7.78 (d, J=4.8 Hz, 1H), 7.57-7.50 (m, 2H), 7.50-7.42 (m, 4H), 7.42-7.36 (m, 1H), 7.20-7.14 (m, 1H), 7.01 (br s, 2H), 6.38 (dd, J=7.6, 4.8 Hz, 1H), 4.34-4.24 (m, 1H), 3.64 (s, 2H), 3.00 (d, J=10.0 Hz, 2H), 2.63-2.56 (m, 2H), 2.21-2.06 (m, 2H), 1.65 (d, J=10.0 Hz, 2H).

### Example 60: N-(1-(4-(2-(2-Aminopyridin-3-yl)-5-phenyl-3H-imidazo[4,5-b]pyridin-3-yl)benzyl)piperidin-4-yl)-4-cyanonicotinamide

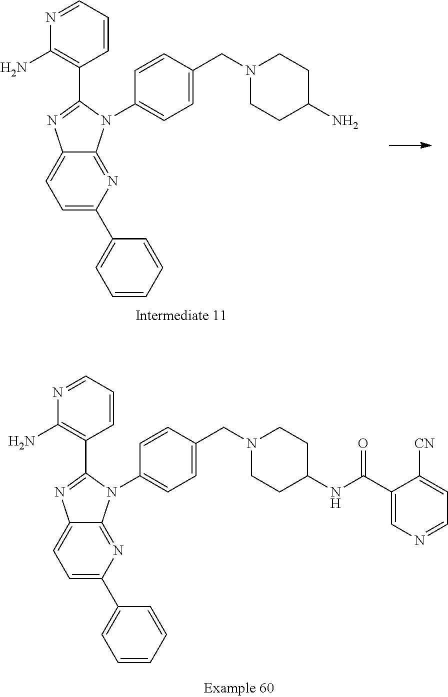 [SMILES: Nc1ncccc1-c1nc2ccc(-c3ccccc3)nc2n1-c1ccc(CN2CCC(N)CC2)cc1 ; N#Cc1ccncc1C(=O)NC1CCN(Cc2ccc(-n3c(-c4cccnc4N)nc4ccc(-c5ccccc5)nc43)cc2)CC1]

Following the general procedure of Example 23, the reaction of Intermediate 11 (200 mg, 421 μmol) with 4-cyanopyridine-3-carboxylic acid (75 mg, 505 μmol). After purified by prep-HPLC (column: Phenomenex C18 150*25 mm*10 μm; mobile phase: [water (NH₄HCO₃)-ACN]; B %: 33%-63%, 8 min), N-(1-(4-(2-(2-aminopyridin-3-yl)-5-phenyl-3H-imidazo[4,5-b]pyridin-3-yl)benzyl)piperidin-4-yl)-4-cyanonicotinamide (Example 60, 58.8 mg, yield: 23%) was obtained as a yellow solid. MS: m/z=606.3 [M+H]⁺. ¹H NMR (400 MHz, Dimethylsulfoxide-d₆) δ 10.60 (s, 1H), 9.10-8.93 (m, 2H), 8.27 (d, J=8.4 Hz, 1H), 8.19-8.12 (m, 1H), 8.03 (d, J=7.2 Hz, 2H), 8.01-7.95 (m, 2H), 7.57-7.49 (m, 2H), 7.49-7.43 (m, 4H), 7.42-7.34 (m, 1H), 7.17 (dd, J=7.6, 1.6 Hz, 1H), 7.03 (br s, 2H), 6.38 (dd, J=7.6, 4.8 Hz, 1H), 4.34-4.21 (m, 1H), 3.62 (s, 2H), 2.98 (d, J=11.2 Hz, 2H), 2.64-2.52 (m, 2H), 2.09 (t, 0.1=11.2 Hz, 2H), 1.69-1.56 (m, 2H).

### Example 61: N-(1-(4-(2-(2-Aminopyridin-3-yl)-5-phenyl-3H-imidazo[4,5-b]pyridin-3-yl)benzyl)piperidin-4-yl)-3-cyanopicolinamide

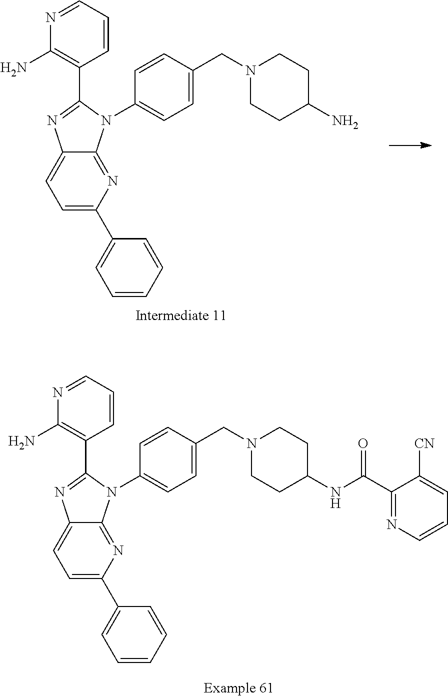 [SMILES: Nc1ncccc1-c1nc2ccc(-c3ccccc3)nc2n1-c1ccc(CN2CCC(N)CC2)cc1 ; N#Cc1cccnc1C(=O)NC1CCN(Cc2ccc(-n3c(-c4cccnc4N)nc4ccc(-c5ccccc5)nc43)cc2)CC1]

Following the general procedure of Example 23, the reaction of Intermediate 11 (200 mg, 421 μmol) with and 3-cyanopyridine-2-carboxylic acid (75 mg, 505 μmol) was carried out. After purified by prep-HPLC (column: Phenomenex C18 150*25 mm*10 μm; mobile phase: [water (NH₄HCO₃)-ACN]; B %: 36%-66%, 8 min), N-(1-(4-(2-(2-aminopyridin-3-yl)-5-phenyl-3H-imidazo[4,5-b]pyridin-3-yl)benzyl)piperidin-4-yl)-3-cyanopicolinamide (Example 61, 25.2 mg, yield: 10%) was obtained as an off-white solid. MS: m/z=606.1 [M+H]⁺. ¹H NMR (400 MHz, Dimethylsulfoxide-d₆) δ 10.45 (s, 1H), 8.87 (dd, J=4.8, 1.2 Hz, 1H), 8.55 (dd, J=7.6, 1.2 Hz, 1H), 8.27 (d, J=8.4 Hz, 1H), 8.04 (d, J=7.2 Hz, 2H), 8.01-7.97 (m, 2H), 7.76 (dd, J=7.6, 4.8 Hz, 1H), 7.55-7.50 (m, 2H), 7.49-7.45 (m, 4H), 7.41-7.37 (m, 1H), 7.17 (dd, J=7.6, 1.6 Hz, 1H), 7.04 (br s, 2H), 6.38 (dd, J=7.6, 4.8 Hz, 1H), 4.38-4.30 (m, 1H), 3.63 (s, 2H), 2.99 (d, J=10.8 Hz, 2H), 2.66-2.52 (m, 2H), 2.15-2.06 (m, 2H), 1.65 (d, J=10.0 Hz, 2H).

### Example 62: 4-((1-(4-(2-(2-Aminopyridin-3-yl)-3H-imidazo[4,5-b]pyridin-3-yl)benzyl)piperidin-4-yl)amino)picolinonitrile

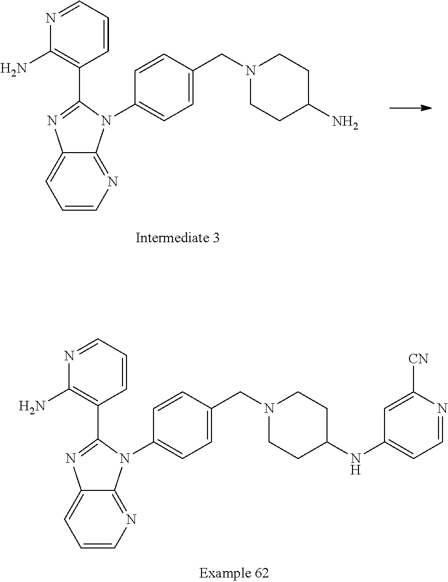 [SMILES: Nc1ncccc1-c1nc2cccnc2n1-c1ccc(CN2CCC(N)CC2)cc1 ; N#Cc1cc(NC2CCN(Cc3ccc(-n4c(-c5cccnc5N)nc5cccnc54)cc3)CC2)ccn1]

To a solution of Intermediate 3 (200 mg, 501 μmol) and 4-fluoropyridine-2-carbonitrile (73 mg, 601 μmol) in DMSO (2 mL) was added DIEA (324 mg, 2.50 mmol). The mixture was stirred at 100° C. for 16 hr. Then the mixture was purified by prep-HPLC (column: Phenomenex C18 150*25 mm*10 μm; mobile phase: [water (NH₄HCO₃)-ACN]; B %: 28%-58%, 8 min), 4-((1-(4-(2-(2-aminopyridin-3-yl)-3H-imidazo[4,5-b]pyridin-3-yl)benzyl)piperidin-4-yl)amino)picolinonitrile (Example 62, 23.8 mg, yield: 9.5%) as a light-yellow solid. MS: m/z=502.4 [M+H]⁺. ¹H NMR (400 MHz, Dimethylsulfoxide-d₆) δ 8.34 (dd, J=4.8, 1.2 Hz, 1H), 8.21 (dd, J=8.0, 1.2 Hz, 1H), 8.09 (d, J=5.6 Hz, 1H), 8.03-7.95 (m, 1H), 7.46 (d, J=8.4 Hz, 2H), 7.44-7.36 (m, 3H), 7.17 (dd, J=8.0, 2.0 Hz, 1H), 7.12-7.04 (m, 2H), 7.01 (br s, 2H), 6.75 (dd, J=6.0, 2.4 Hz, 1H), 6.38 (dd, J=7.6, 4.8 Hz, 1H), 3.57 (s, 2H), 2.86-2.77 (m, 3H), 2.20-2.11 (m, 2H), 1.93-1.85 (m, 2H), 1.50-1.37 (m, 2H).

### Example 63: 6-((1-(4-(2-(2-Aminopyridin-3-yl)-3H-imidazo[4,5-b]pyridin-3-yl)benzyl)piperidin-4-yl)amino)picolinonitrile

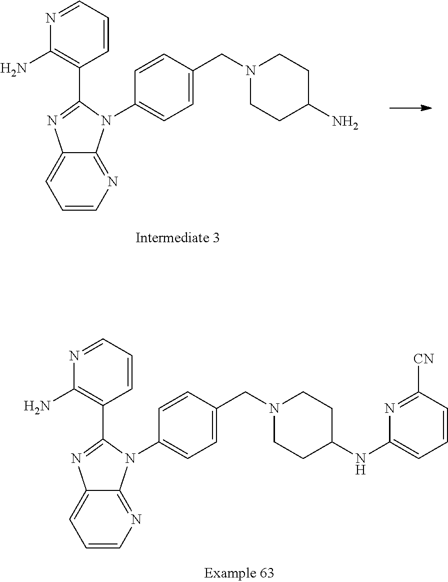 [SMILES: Nc1ncccc1-c1nc2cccnc2n1-c1ccc(CN2CCC(N)CC2)cc1 ; N#Cc1cccc(NC2CCN(Cc3ccc(-n4c(-c5cccnc5N)nc5cccnc54)cc3)CC2)n1]

Following the general procedure of Example 62, the reaction of Intermediate 3 (200 mg, 501 μmol) with 6-fluoropyridine-2-carbonitrile (73 mg, 601 μmol) was carried out. After purified by prep-HPLC (column: Phenomenex C18 150*25 mm*10 μm; mobile phase: [water (NH₄HCO₃)-ACN]; B %: 22%-52%, 8 min), 6-((1-(4-(2-(2-aminopyridin-3-yl)-3H-imidazo[4,5-b]pyridin-3-yl)benzyl)piperidin-4-yl)amino)picolinonitrile (Example 63, 15.0 mg, yield: 6.0%) was obtained as a light-green solid. MS: m/z=502.2 [M+H]⁺. ¹H NMR (400 MHz, Dimethylsulfoxide-d₆) δ 8.34 (d, J=4.8 Hz, 1H), 8.21 (d, J=8.0 Hz, 1H), 8.05-7.95 (m, 1H), 7.54-7.44 (m, 3H), 7.43-7.34 (m, 3H), 7.17 (d, J=7.2 Hz, 1H), 7.11 (d, J=7.6 Hz, 1H), 7.08-6.98 (m, 3H), 6.77 (d, J=8.4 Hz, 1H), 6.40-6.37 (m, 1H), 3.72-3.67 (m, 1H), 3.57 (br s, 2H), 2.84-2.79 (m, 2H), 2.19-2.10 (m, 2H), 1.94-1.87 (m, 2H), 1.51-1.41 (m, 2H).

### Example 64: 6-((1-(4-(2-(2-aminopyridin-3-yl)-3H-imidazo[4,5-b]pyridin-3-yl)benzyl)piperidin-4-yl)amino)pyrimidine-4-carbonitrile

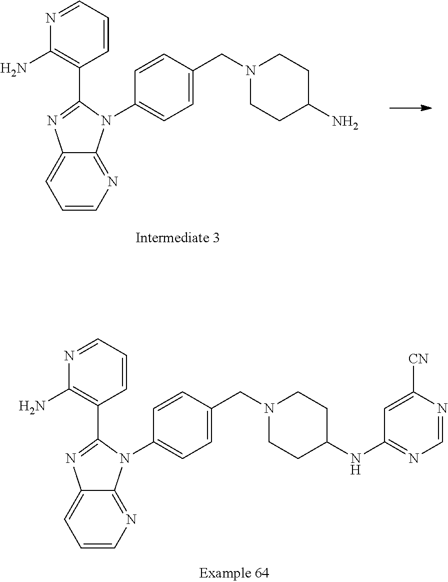 [SMILES: Nc1ncccc1-c1nc2cccnc2n1-c1ccc(CN2CCC(N)CC2)cc1 ; N#Cc1cc(NC2CCN(Cc3ccc(-n4c(-c5cccnc5N)nc5cccnc54)cc3)CC2)ncn1]

Following the general procedure of Example 62, the reaction of Intermediate 3 (250 mg, 626 μmol) with 6-chloropyrimidine-4-carbonitrile (105 mg, 751 μmol) was carried out under microwave 130° C. After purified by prep-HPLC (column: Phenomenex C18 150*25 mm*10 μm; mobile phase: [water (NH₄HCO₃)-ACN]; B %: 20%-50%, 8 min), 6-((1-(4-(2-(2-aminopyridin-3-yl)-3H-imidazo[4,5-b]pyridin-3-yl)benzyl)piperidin-4-yl)amino)pyrimidine-4-carbonitrile (Example 64, 44.2 mg, yield: 14%) was obtained as a yellow solid. MS: m/z=503.1 [M+H]⁺. ¹H NMR (400 MHz, Chloroform-d) δ 8.56 (s, 1H), 8.39 (dd, J=4.8, 1.2 Hz, 1H), 8.10 (dd, J=8.0, 1.2 Hz, 1H), 8.04 (dd, J=4.8, 1.6 Hz, 1H), 7.52 (d, J=7.8 Hz, 2H), 7.36-7.29 (m, 3H), 7.06 (dd, J=7.6, 1.6 Hz, 1H), 6.68 (s, 1H), 6.62 (br s, 2H), 6.33 (dd, J=7.6, 4.8 Hz, 1H), 5.56-5.27 (m, 1H), 4.33-3.84 (m, 1H), 3.66 (s, 2H), 3.01-2.91 (m, 2H), 2.35-2.26 (m, 2H), 2.06 (d, J=11.2 Hz, 2H), 1.71-1.62 (m, 2H).

### Example 65: 5-(4-(4-(2-(2-Aminopyridin-3-yl)-5-phenyl-3H-imidazo[4,5-b]pyridin-3-yl)benzyl)piperazin-1-yl)nicotinonitrile

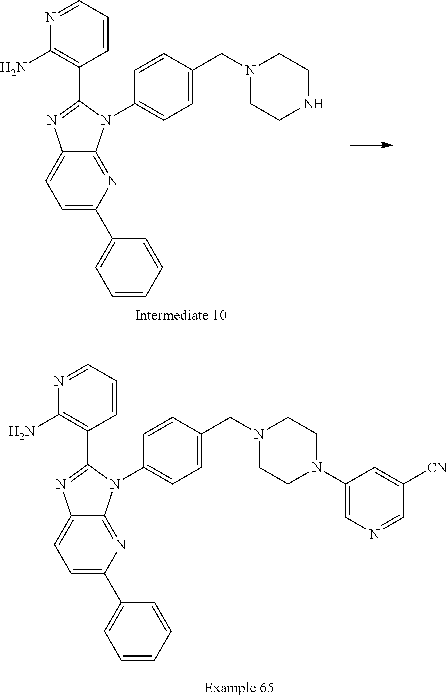 [SMILES: Nc1ncccc1-c1nc2ccc(-c3ccccc3)nc2n1-c1ccc(CN2CCNCC2)cc1 ; N#Cc1cncc(N2CCN(Cc3ccc(-n4c(-c5cccnc5N)nc5ccc(-c6ccccc6)nc54)cc3)CC2)c1]

Following the general procedure of Example 62, the reaction of Intermediate 10 (200 mg, 433 μmol) with 5-fluoropyridine-3-carbonitrile (63.5 mg, 520 μmol) was carried out. After purified by prep-HPLC (column: Phenomenex C18 150*25 mm*10 μm; mobile phase: [water (NH₄HCO₃)-ACN]; B %: 40%-70%, 8 min), 5-(4-(4-(2-(2-aminopyridin-3-yl)-5-phenyl-3H-imidazo[4,5-b]pyridin-3-yl)benzyl)piperazin-1-yl)nicotinonitrile (Example 65, 18.5 mg, yield: 7.6%) was obtained as a yellow solid. MS: m/z=564.1 [M+H]⁺. ¹H NMR (400 MHz, Chloroform-d) δ 8.48 (d, J=3.2 Hz, 1H), 8.30 (d, J=1.6 Hz, 1H), 8.14 (d, J=8.4 Hz, 1H), 8.10-8.04 (m, 1H), 8.02 (d, J=7.2 Hz, 2H), 7.81 (d, J=8.4 Hz, 1H), 7.52 (d, J=8.4 Hz, 2H), 7.45-7.41 (m, 4H), 7.40-7.38 (m, 1H), 7.34-7.32 (m, 1H), 7.12 (dd, J=7.6, 1.6 Hz, 1H), 6.67 (br s, 2H), 6.38 (dd, J=7.6, 4.8 Hz, 1H), 3.69 (s, 2H), 3.35-3.31 (m, 4H), 2.72-2.68 (m, 4H).

### Example 66: 2-(4-(4-(2-(2-Aminopyridin-3-yl)-5-phenyl-3H-imidazo[4,5-b]pyridin-3-yl)benzyl)piperazin-1-yl)isonicotinonitrile

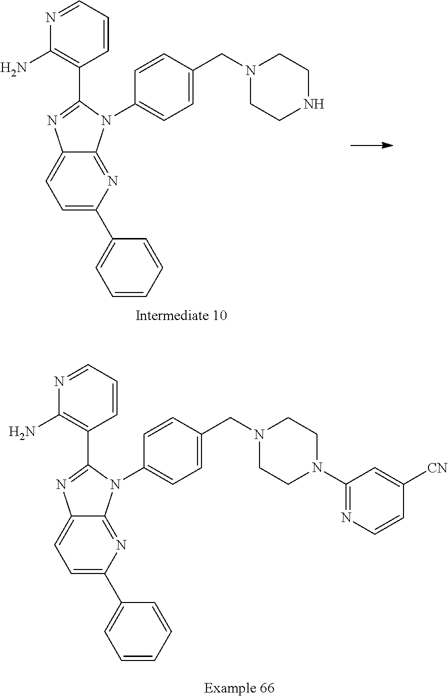 [SMILES: Nc1ncccc1-c1nc2ccc(-c3ccccc3)nc2n1-c1ccc(CN2CCNCC2)cc1 ; N#Cc1ccnc(N2CCN(Cc3ccc(-n4c(-c5cccnc5N)nc5ccc(-c6ccccc6)nc54)cc3)CC2)c1]

Following the general procedure of Example 62, the reaction of Intermediate 10 (150 mg, 325 μmol) with 2-chloropyridine-4-carbonitrile (90 mg, 650 μmol) was carried out under microwave 130° C. After purified by prep-HPLC (column: Waters xbridge 150*25 mm 10 μm; mobile phase: [water (NH₄HCO₃)-ACN]; B %: 51%-81%, 8 min), 2-(4-(4-(2-(2-aminopyridin-3-yl)-5-phenyl-3H-imidazo[4,5-b]pyridin-3-yl)benzyl)piperazin-1-yl)isonicotinonitrile (Example 66, 16.6 mg, yield: 9.1%) was obtained as a yellow solid. MS: m/z=564.2 [M+H]⁺. ¹H NMR (400 MHz, Chloroform-d) δ 8.28 (d, J=5.2 Hz, 1H), 8.14 (d, J=8.4 Hz, 1H), 8.07 (dd, J=4.8, 1.6 Hz, 1H), 8.02 (d, J=6.8 Hz, 2H), 7.81 (d, J=8.4 Hz, 1H), 7.52 (d, J=8.4 Hz, 2H), 7.49-7.41 (m, 4H), 7.40-7.35 (m, 1H), 7.10 (dd, J=7.6, 1.6 Hz, 1H), 6.82 (s, J H), 6.76 (dd, J=5.2, 1.2 Hz, 1H), 6.61 (br s, 2H), 6.36 (dd, J=7.6, 4.8 Hz, 1H), 3.66 (s, 2H), 3.66-3.58 (m, 4H), 2.68-2.56 (m, 4H).

### Example 67:6-(4-(4-(2-(2-Aminopyridin-3-yl)-5-phenyl-3H-imidazo[4,5-b]pyridin-3-yl)benzyl)piperazin-1-yl)pyrazine-2-carbonitrile

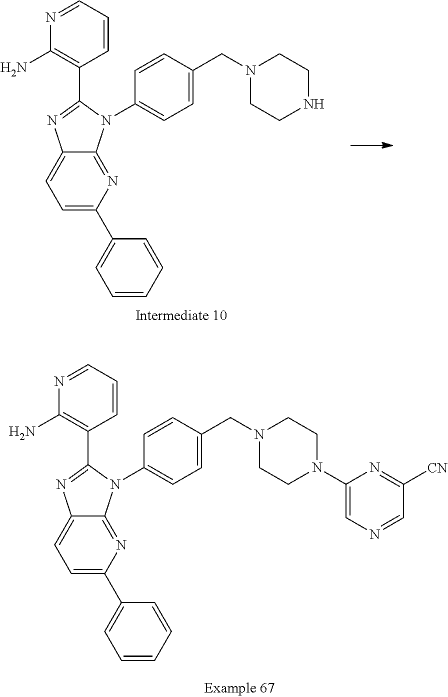 [SMILES: Nc1ncccc1-c1nc2ccc(-c3ccccc3)nc2n1-c1ccc(CN2CCNCC2)cc1 ; N#Cc1cncc(N2CCN(Cc3ccc(-n4c(-c5cccnc5N)nc5ccc(-c6ccccc6)nc54)cc3)CC2)n1]

Following the general procedure of Example 62, the reaction of Intermediate 10 (150 mg, 325 μmol) with 6-chloropyrazine-2-carbonitrile (91 mg, 650 μmol) was carried out under microwave 130° C. After purified by prep-HPLC (column: Waters xbridge 150*25 mm 10 μm; mobile phase: [water (NH₄HCO₃)-ACN]; B %: 18%-48%, 8 min), 6-(4-(4-(2-(2-aminopyridin-3-yl)-5-phenyl-3H-imidazo[4,5-b]pyridin-3-yl)benzyl)piperazin-1-yl)pyrazine-2-carbonitrile (Example 67, 25.6 mg, 14% yield) was obtained as a yellow solid. MS: m/z=565.4 [M+H]⁺. ¹H NMR (400 MHz, Chloroform-d) δ 8.29 (s, 1H), 8.18-8.10 (m, 2H), 8.08 (d, J=4.8 Hz, 1H), 8.02 (d, J=8.0 Hz, 2H), 7.81 (d, J=8.4 Hz, 1H), 7.52 (d, J=8.0 Hz, 2H), 7.47-7.42 (m, 4H), 7.41-4.35 (m, 1H), 7.11 (d, J=7.6 Hz, 1H), 6.66 (br s, 2H), 6.38 (dd, J=7.6, 4.8 Hz, 1H), 3.74-3.69 (m, 4H), 3.67 (s, 2H), 2.63 (t, J=4.8 Hz, 4H).

### Example 68: 2-(4-(4-(2-(2-Aminopyridin-3-yl)-5-phenyl-3H-imidazo[4,5-b]pyridin-3-yl)benzyl)piperazin-1-yl)pyrimidine-4-carbonitrile

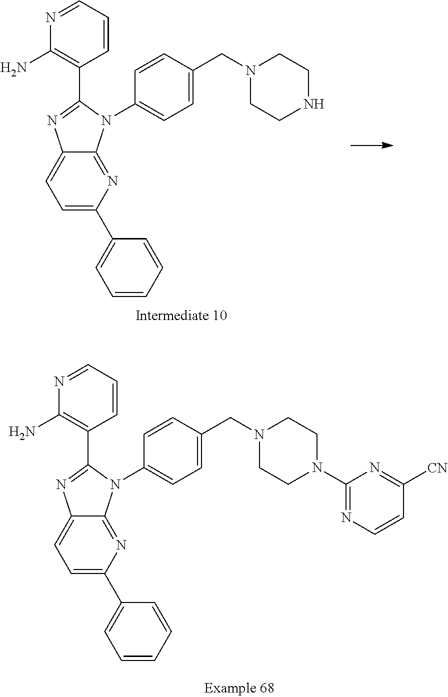 [SMILES: Nc1ncccc1-c1nc2ccc(-c3ccccc3)nc2n1-c1ccc(CN2CCNCC2)cc1 ; N#Cc1ccnc(N2CCN(Cc3ccc(-n4c(-c5cccnc5N)nc5ccc(-c6ccccc6)nc54)cc3)CC2)n1]

Following the general procedure of Example 62, the reaction of Intermediate 10 (200 mg, 433 μmol) with 2-chloropyridine-4-carbonitrile (72 mg, 520 μmol) was carried out under microwave 130° C. After purified by prep-HPLC (column: Waters xbridge 150*25 mm 10 μm; mobile phase: [water (NH₄HCO₃)-ACN]; B %: 50%-80%, 8 min), 2-(4-(4-(2-(2-aminopyridin-3-yl)-5-phenyl-3H-imidazo[4,5-b]pyridin-3-yl)benzyl)piperazin-1-yl)pyrimidine-4-carbonitrile (Example 68, 31.0 mg, yield: 13%) was obtained as a yellow solid. MS: m/z=565.2 [M+H]⁺. ¹H NMR (400 MHz, Chloroform-d) δ 8.45 (d, J=4.8 Hz, 1H), 8.14 (d, J=8.4 Hz, 1H), 8.07 (d, J=4.4 Hz, 1H), 8.02 (d, J=7.6 Hz, 2H), 7.81 (d, J=8.4 Hz, 1H), 7.53 (d, J=8.0 Hz, 2H), 7.48-7.41 (m, 4H), 7.41-7.36 (m, 1H), 7.11 (d, J=7.6 Hz, 1H), 6.75 (d, J=4.8 Hz, 1H), 6.67 (br s, 2H), 6.37 (dd, J=7.6, 4.8 Hz, 1H), 3.94-3.87 (m, 4H), 3.66 (s, 2H), 2.58 (t, J=4.8 Hz, 4H).

### Example 69: 6-(4-(4-(2-(2-Aminopyridin-3-yl)-3H-imidazo[4,5-b]pyridin-3-yl)benzyl)piperazine-1-carbonyl)picolinonitrile

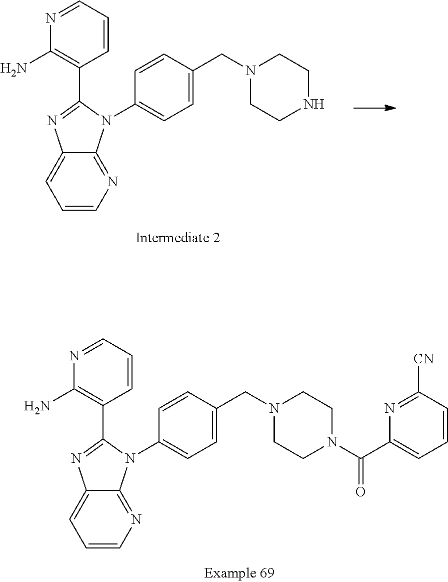 [SMILES: Nc1ncccc1-c1nc2cccnc2n1-c1ccc(CN2CCNCC2)cc1 ; N#Cc1cccc(C(=O)N2CCN(Cc3ccc(-n4c(-c5cccnc5N)nc5cccnc54)cc3)CC2)n1]

To a solution of Intermediate 2 and 6-cyanopicolinic acid (84.5 mg, 570 μmol) in DMF (3 mL) was added HATU (395 mg, 1.04 mmol) and DIEA (201 mg, 1.56 mmol), then the reaction mixture was stirred at 25° C. for 4 hr. The reaction mixture was poured into H₂O (50 mL). The resulting mixture was extracted with CH₂Cl₂ (50 mL×3). The combined organic dried over anhydrous Na₂SO₄, filtered and concentrated to dryness. The residue was purified by prep-HPLC (column: Waters xbridge 150*40 mm 15 μm; mobile phase: [water (NH₄HCO₃)-ACN]; B %: 26%-56%, 8 min), 6-(4-(4-(2-(2-aminopyridin-3-yl)-3H-imidazo[4,5-b]pyridin-3-yl)benzyl)piperazine-1-carbonyl)picolinonitrile (Example 69, 36.6 mg, yield: 14%) was obtained as an off-white lyophilized powder. MS: m/z=516.1 [M+H]⁺. ¹H NMR (400 MHz, Dimethylsulfoxide-d₆) δ 8.33 (dd, J=4.8, 1.6 Hz, 1H), 8.25-8.16 (m, 2H), 8.15-8.09 (m, 1H), 7.98 (dd, J=4.8, 2.0 Hz, 1H), 7.90 (dd, J=7.6, 1.2 Hz, 1H), 7.47 (d, J=8.4 Hz, 2H), 7.43-7.32 (m, 3H), 7.16 (dd, J=7.6, 1.6 Hz, 1H), 7.00 (br s, 2H), 6.38 (dd, J=8.0, 5.2 Hz, 1H), 3.73-3.65 (m, 2H), 3.62 (s, 2H), 3.41-3.36 (m, 2H), 2.47-2.36 (m, 4H).

### Example 70: 4-(4-(4-(2-(2-Aminopyridin-3-yl)-5-phenyl-3H-imidazo[4,5-b]pyridin-3-yl)benzyl)piperazine-1-carbonyl)pyridazine-3-carbonitrile

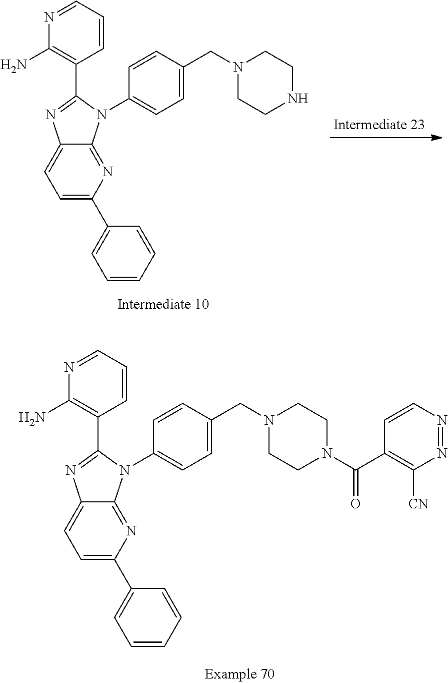 [SMILES: Nc1ncccc1-c1nc2ccc(-c3ccccc3)nc2n1-c1ccc(CN2CCNCC2)cc1 ; N#Cc1nnccc1C(=O)N1CCN(Cc2ccc(-n3c(-c4cccnc4N)nc4ccc(-c5ccccc5)nc43)cc2)CC1]

Following the general procedure of Example 69, the reaction of Intermediate 10 (200 mg, 433 μmol) with Intermediate 23 (71 mg, 476 μmol) was carried out. After purified by prep-HPLC (column: Waters xbridge 150*25 mm 10 μm; mobile phase: [water (NH₄HCO₃)-ACN]; B %: 35%-65%, 10 min), 4-(4-(4-(2-(2-aminopyridin-3-yl)-5-phenyl-3H-imidazo[4,5-b]pyridin-3-yl)benzyl)piperazine-1-carbonyl)pyridazine-3-carbonitrile (Example 70, 14.1 mg, yield: 5.5%) was obtained as a yellow solid. MS: m/z=593.3 [M+H]⁺. ¹H NMR (400 MHz, Dimethylsulfoxide-d₆) δ 9.61 (d, J=5.2 Hz, 1H), 8.27 (d, J=8.4 Hz, 1H), 8.07 (d, J=5.2 Hz, 1H), 8.05-8.01 (m, 2H), 8.00-7.96 (m, 2H), 7.54-7.44 (m, 6H), 7.42-7.36 (m, 1H), 7.15 (dd, J=7.6, 1.6 Hz, 1H), 7.01 (br s, 2H), 6.38 (dd, J=7.6, 4.8 Hz, 1H), 3.76-3.70 (m, 2H), 3.65 (s, 2H), 3.37-3.31 (m, 2H), 2.58-2.52 (m, 2H), 2.46-2.40 (m, 2H).

### Example 71: N-(1-(4-(2-(2-Aminopyridin-3-yl)-5-phenyl-3H-imidazo[4,5-b]pyridin-3-yl)benzyl)piperidin-4-yl)-3-cyanopyridazine-4-carboxamide

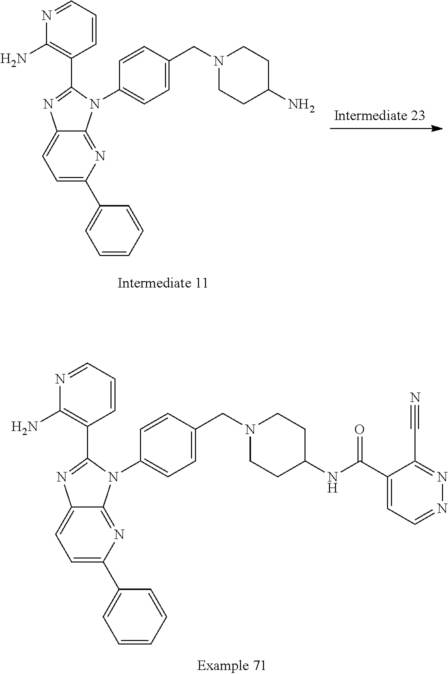 [SMILES: Nc1ncccc1-c1nc2ccc(-c3ccccc3)nc2n1-c1ccc(CN2CCC(N)CC2)cc1 ; N#Cc1nnccc1C(=O)NC1CCN(Cc2ccc(-n3c(-c4cccnc4N)nc4ccc(-c5ccccc5)nc43)cc2)CC1]

Following the general procedure of Example 69, the reaction of Intermediate 11 (200 mg, 420 μmol) with Intermediate 23 (69 mg, 462 μmol) was carried out. The residue was purified by silica gel flash chromatography (Eluent of 0˜7% MeOH in CH₂Cl₂), N-(1-(4-(2-(2-aminopyridin-3-yl)-5-phenyl-3H-imidazo[4,5-b]pyridin-3-yl)benzyl)piperidin-4-yl)-3-cyanopyridazine-4-carboxamide (Example 71, 22.6 mg, yield: 8.9%) was obtained as a yellow solid. MS: m/z=607.4 [M+H]⁺. ¹H NMR (400 MHz, Dimethylsulfoxide-d₆) δ 10.52 (s, 1H), 9.59 (d, J=5.6 Hz, 1H), 8.27 (d, J=8.4 Hz, 1H), 8.14 (d, J=5.2 Hz, 1H), 8.05-7.97 (m, 4H), 7.54-7.44 (m, 6H), 7.41-7.35 (m, 1H), 7.17 (d, J=7.6 Hz, 1H), 7.04 (br s, 2H), 6.38 (dd, 1=8.0, 4.4 Hz, 1H), 4.42-4.28 (m, 1H), 3.63 (s, 2H), 2.99 (d, J=9.6 Hz, 2H), 2.64-2.56 (m, 2H), 2.14-2.08 (m, 2H), 1.70 (d, J=10.4 Hz, 2H).

### Example 72: 4-(4-(4-(2-(2-Aminopyridin-3-yl)-5-phenyl-3H-imidazo[4,5-b]pyridin-3-yl)benzyl)piperazine-1-carbonyl)pyrimidine-5-carbonitrile

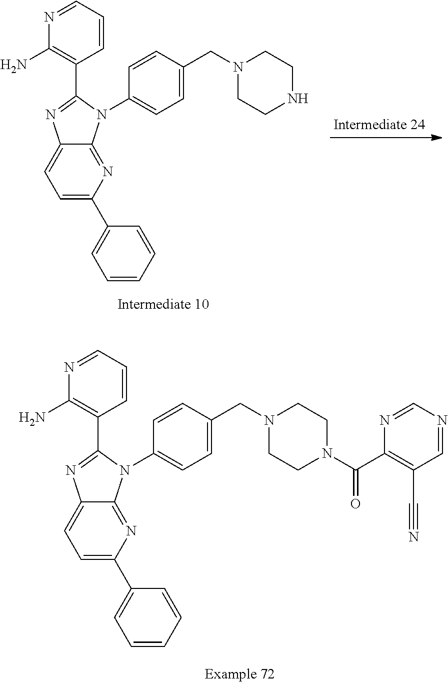 [SMILES: Nc1ncccc1-c1nc2ccc(-c3ccccc3)nc2n1-c1ccc(CN2CCNCC2)cc1 ; N#Cc1cncnc1C(=O)N1CCN(Cc2ccc(-n3c(-c4cccnc4N)nc4ccc(-c5ccccc5)nc43)cc2)CC1]

Following the general procedure of Example 69, the reaction of Intermediate 10 (150 mg, 325 μmol) with Intermediate 24 (48 mg, 325 μmol) was carried out. The residue was purified by silica gel flash chromatography (Eluent of 0˜7% MeOH in CH₂Cl₂), 4-(4-(4-(2-(2-aminopyridin-3-yl)-5-phenyl-3H-imidazo[4,5-b]pyridin-3-yl)benzyl)piperazine-1-carbonyl)pyrimidine-5-carbonitrile (Example 72, 44.8 mg, yield: 23%) was obtained as a yellow solid. MS: m/z=593.4 [M+H]⁺. ¹H NMR (400 MHz, Dimethylsulfoxide-d₆) δ 9.48 (d, J=5.2 Hz, 2H), 8.27 (d, J=8.4 Hz, 1H), 8.05-7.95 (m, 4H), 7.54-7.44 (m, 6H), 7.41-7.35 (m, 1H), 7.14 (dd, J=8.0, 1.6 Hz, 1H), 7.02 (br s, 2H), 6.38 (dd, J=7.6, 4.8 Hz, 1H), 3.77-3.71 (m, 2H), 3.65 (s, 2H), 3.39-3.31 (m, 2H), 2.59-2.51 (m, 2H), 2.48-2.42 (m, 2H).

### Example 73: N-(1-(4-(2-(2-Aminopyridin-3-yl)-5-phenyl-3H-imidazo[4,5-b]pyridin-3-yl)benzyl)piperidin-4-yl)-5-cyanopyrimidine-4-carboxamide

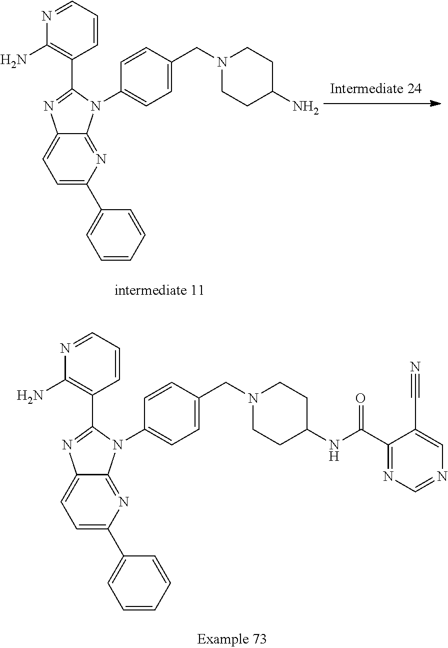 [SMILES: Nc1ncccc1-c1nc2ccc(-c3ccccc3)nc2n1-c1ccc(CN2CCC(N)CC2)cc1 ; N#Cc1cncnc1C(=O)NC1CCN(Cc2ccc(-n3c(-c4cccnc4N)nc4ccc(-c5ccccc5)nc43)cc2)CC1]

Following the general procedure of Example 69, the reaction of Intermediate 11 (150 mg, 315 μmol) with Intermediate 24 (51 mg, 346 μmol) was carried out. After purified by silica gel flash chromatography (Eluent of 0˜7% MeOH in CH₂Cl₂), N-(1-(4-(2-(2-aminopyridin-3-yl)-5-phenyl-3H-imidazo[4,5-b]pyridin-3-yl)benzyl)piperidin-4-yl)-5-cyanopyrimidine-4-carboxamide (Example 73, 34.0 mg, yield: 18%) was obtained as a yellow solid. MS: m/z=607.3 [M+H]⁺. ¹H NMR (400 MHz, Dimethylsulfoxide-d₆) δ 9.62-9.43 (m, 2H), 9.09 (d, J=8.0 Hz, 1H), 8.27 (d, J=8.4 Hz, 1H), 8.05-7.97 (m, 4H), 7.54-7.42 (m, 6H), 7.41-7.35 (m, 1H), 7.17 (d, J=7.2 Hz, 1H), 7.03 (br s, 2H), 6.38 (dd, J=7.6, 4.8 Hz, 1H), 3.90-3.76 (m, 1H), 3.59 (s, 2H), 2.95-2.79 (m, 2H), 2.16-2.06 (m, 2H), 1.85-1.62 (m, 4H).

### Example 74: 5-((1-(4-(2-(2-Aminopyridin-3-yl)-3H-imidazo[4,5-b]pyridin-3-yl)benzyl)piperidin-4-yl)amino)nicotinonitrile

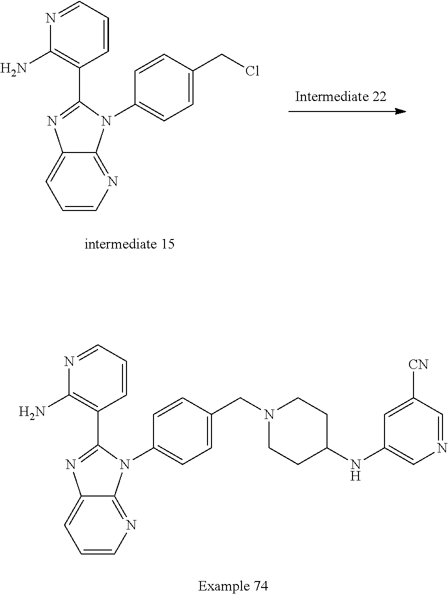 [SMILES: Nc1ncccc1-c1nc2cccnc2n1-c1ccc(CCl)cc1 ; N#Cc1cncc(NC2CCN(Cc3ccc(-n4c(-c5cccnc5N)nc5cccnc54)cc3)CC2)c1]

To a solution of Intermediate 15 and Intermediate 22 (90.4 mg, 447 μmol) in DMF (2 mL) was added K₂CO₃ (124 mg, 893 μmol) and NaI (7 mg, 45 μmol). The mixture was stirred at 80° C. for 2 hr. The reaction mixture was diluted with 20 mL H₂O and extracted with EtOAc (20 mL×2). The combined organic layers were washed with 30 mL brine, dried over Na₂SO₄, filtered and concentrated under reduced pressure to give a residue The residue was purified by prep-HPLC (column: Waters xbridge 150*25 mm 10 μm; mobile phase: [water (NH₄HCO₃)-ACN]; B %: 20%-50%, 11 min), 5-((1-(4-(2-(2-aminopyridin-3-yl)-3H-imidazo[4,5-b]pyridin-3-yl)benzyl)piperidin-4-yl)amino)nicotinonitrile (Example 74, 15 mg, yield: 6.3%) was obtained as a light-yellow solid. MS: m/z=502.4 [M+H]⁺. ¹H NMR (400 MHz, Dimethylsulfoxide-d₆) δ 8.33 (dd, J=4.4, 1.2 Hz, 1H), 8.23-8.19 (m, 2H), 8.06 (d, J=1.6 Hz, 1H), 7.98 (dd, J=4.8, 2.0 Hz, 1H), 7.45 (d, J=8.0, 2H), 7.43-7.35 (m, 4H), 7.33-7.31 (m, 1H), 7.16 (dd, J=7.6, 1.6 Hz, 1H), 7.00 (br s, 2H), 6.37 (dd, J=7.6, 4.8 Hz, 1H), 3.57 (s, 2H), 2.83-2.78 (m, 2H), 2.19-2.16 (m, 2H), 1.93-1.88 (m, 2H), 1.46-1.38 (m, 2H).

### Example 75: 2-((1-(4-(2-(2-Aminopyridin-3-yl)-3H-imidazo[4,5-b]pyridin-3-yl)benzyl)piperidin-4-yl)amino)isonicotinonitrile

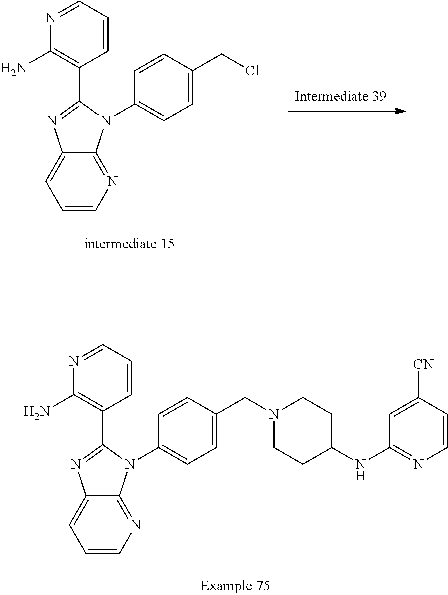 [SMILES: Nc1ncccc1-c1nc2cccnc2n1-c1ccc(CCl)cc1 ; N#Cc1ccnc(NC2CCN(Cc3ccc(-n4c(-c5cccnc5N)nc5cccnc54)cc3)CC2)c1]

To a solution of Intermediate 15 (150 mg, 446 μmol) in DMF (2 mL) was added NaI (6.7 mg, 44.7 μmol), K₂CO₃ (124 mg, 893 μmol) and Intermediate 39 (108 mg, 536 μmol). The mixture was stirred at 80° C. for 2 hr. The reaction mixture was quenched with H₂O (5 mL) at 25° C., and then diluted with CH₂Cl₂ (5 mL) and extracted with CH₂Cl₂ (10 mL×3). The combined organic layers were washed with brine (20 mL), dried over Na₂SO₄, filtered, and concentrated under reduced pressure to give a residue. The crude product was purified by prep-HPLC (column: Phenomenex C18 150×25 mm×10 μm; mobile phase: [water (NH₄HCO₃)-ACN]; B %: 26%-56%, 8 min), 2-((1-(4-(2-(2-aminopyridin-3-yl)-3H-imidazo[4,5-b]pyridin-3-yl)benzyl)piperidin-4-yl)amino)isonicotinonitrile (Example 75, 25.9 mg, yield: 12%) was obtained as a yellow solid. MS: m/z=502.1 [M+H]⁺. ¹H NMR (400 MHz, Dimethylsulfoxide-d₆) δ 8.33 (d, J=4.8 Hz, 1H), 8.21 (dd, J=8.0, 1.2 Hz, 1H), 8.15 (d, J=5.2 Hz, 1H), 7.99 (dd, J=5.2, 1.6 Hz, 1H), 7.45 (d, J=8.4 Hz, 2H), 7.42-7.35 (m, 3H), 7.16 (dd, J=7.6, 2.0 Hz, 1H), 7.05 (d, J=7.6 Hz, 1H), 7.01 (br s, 2H), 6.80 (s, 1H), 6.75 (d, J=5.2 Hz, 1H), 6.437 (dd, J=7.6, 4.8 Hz, 1H), 3.79-3.66 (m, 1H), 3.55 (s, 2H), 2.86-2.76 (m, 2H), 2.18-2.05 (m, 2H), 1.91-1.81 (m, 2H), 1.53-1.39 (m, 2H).

### Example 76: 6-((1-(4-(2-(2-Aminopyridin-3-yl)-3H-imidazo[4,5-b]pyridin-3-yl)benzyl)piperidin-4-yl)amino)pyrazine-2-carbonitrile

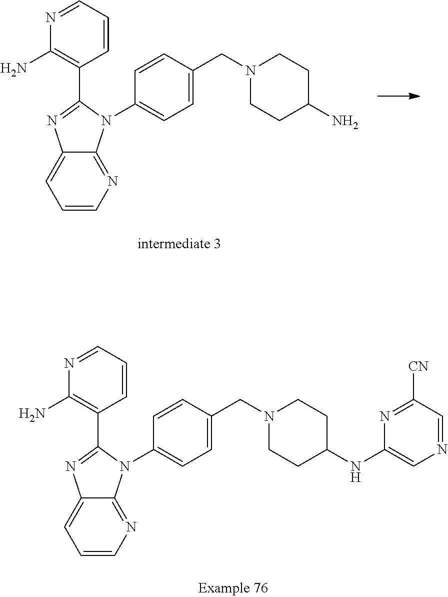 [SMILES: Nc1ncccc1-c1nc2cccnc2n1-c1ccc(CN2CCC(N)CC2)cc1 ; N#Cc1cncc(NC2CCN(Cc3ccc(-n4c(-c5cccnc5N)nc5cccnc54)cc3)CC2)n1]

To a solution of Intermediate 3 (200 mg, 501 μmol) and 6-chloropyrazine-2-carbonitrile (84 mg, 601 μmol) in NMP (2 mL) was added DIEA (324 mg, 2.5 mmol). The mixture was stirred under microwave at 130° C. for 0.5 hr. Then the mixture was purified by prep-HPLC (column: Waters Xbridge 150*25 mm*5 μm; mobile phase: [water (NH₄HCO₃)-ACN]; B %: 30%-60%, 8 min), 6-((1-(4-(2-(2-aminopyridin-3-yl)-3H-imidazo[4,5-b]pyridin-3-yl)benzyl)piperidin-4-yl)amino)pyrazine-2-carbonitrile (Example 76, 12.6 mg, yield: 5.0%) as light-pink solid. MS: m/z=503.2 [M+H]⁺. ¹H NMR (400 MHz, Dimethylsulfoxide-d₆) δ 8.33 (d, J=4.8 Hz, 1H), 8.21 (d, J=8.0 Hz, 1H), 8.09 (d, J=10.4 Hz, 2H), 7.98 (d, J=4.8 Hz, 1H), 7.80-7.71 (m, 1H), 7.45 (m, J=7.6 Hz, 2H), 7.42-7.36 (m, 3H), 7.16 (d, J=8.4 Hz, 1H), 7.01 (br s, 2H), 6.37 (dd, J=7.6, 4.8 Hz, 1H), 3.71-3.68 (m, 1H), 3.57 (s, 2H), 2.83-2.79 (m, 2H), 2.18-2.12 (m, 2H), 1.94-1.88 (m, 2H), 1.53-1.44 (m, 2H).

### Example 77: 2-((1-(4-(2-(2-Aminopyridin-3-yl)-3H-imidazo[4,5-b]pyridin-3-yl)benzyl)piperidin-4-yl)amino)pyrimidine-4-carbonitrile

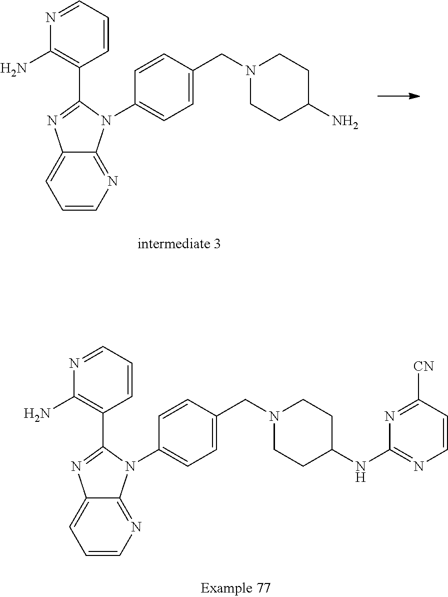 [SMILES: Nc1ncccc1-c1nc2cccnc2n1-c1ccc(CN2CCC(N)CC2)cc1 ; N#Cc1ccnc(NC2CCN(Cc3ccc(-n4c(-c5cccnc5N)nc5cccnc54)cc3)CC2)n1]

Following the general procedure of Example 76, the reaction of Intermediate 3 (200 mg, 501 μmol) with 2-chloropyrimidine-4-carbonitrile (84 mg, 601 μmol) was carried out. After purified by prep-HPLC (column: Phenomenex C18 150*25 mm*10 μm; mobile phase: [water (NH₄HCO₃)-ACN]; B %: 25%-55%, 8 min) and then (column. Phenomenex luna C18 150*25 mm*10 μm; mobile phase: [water (FA)-ACN]; B %: 0%-30%, 58 min), 2-((1-(4-(2-(2-aminopyridin-3-yl)-3H-imidazo[4,5-b]pyridin-3-yl)benzyl)piperidin-4-yl)amino)pyrimidine-4-carbonitrile (Example 77, 5.7 mg, 3 HCOOH salt, yield: 2.3%) was obtained as an off-white solid. MS: m/z=503.3 [M+H]⁺. ¹H NMR (400 MHz, Methanol-d₄) δ 8.49 (d, J=4.8 Hz, 1H), 8.35 (d, J=4.8 Hz, 1H), 8.24-8.16 (m, 4H), 8.02-7.95 (m, 1H), 7.68 (d, J=8.4 Hz, 2H), 7.54 (d, J=8.0 Hz, 2H), 7.45 (dd, J=8.0, 4.8 Hz, 1H), 7.39-7.33 (m, 1H), 6.96 (d, J=4.4 Hz, 1H), 6.49 (dd, J=7.6, 4.8 Hz, 1H), 4.33 (br s, 2H), 4.11-4.03 (m, 1H), 3.47 (d, J=12.4 Hz, 2H), 3.11 (t, J=11.2 Hz, 2H), 2.24 (d, J=12.4 Hz, 2H), 1.94-1.80 (m, 2H).

### Example 78: 6-(4-(4-(2-(2-Aminopyridin-3-yl)-5-phenyl-3H-imidazo[4,5-b]pyridin-3-yl)benzyl)piperazin-1-yl)pyrimidine-4-carbonitrile

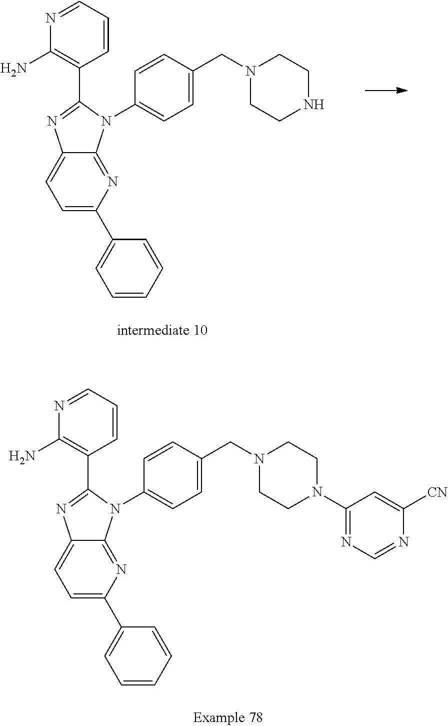 [SMILES: Nc1ncccc1-c1nc2ccc(-c3ccccc3)nc2n1-c1ccc(CN2CCNCC2)cc1 ; N#Cc1cc(N2CCN(Cc3ccc(-n4c(-c5cccnc5N)nc5ccc(-c6ccccc6)nc54)cc3)CC2)ncn1]

Following the general procedure of Example 76, the reaction of Intermediate 10 (150 mg, 325 μmol) with 6-chloropyrimidine-4-carbonitrile (68 mg, 487 μmol) was carried out. After purified by prep-HPLC (column: Waters xbridge 150*25 m 10 μm; mobile phase. [water (NH₄HCO₃)-ACN]; B %: 44%-74%, 8 min), 6-(4-(4-(2-(2-aminopyridin-3-yl)-5-phenyl-3H-imidazo[4,5-b]pyridin-3-yl)benzyl)piperazin-1-yl)pyrimidine-4-carbonitrile (Example 78, 21.1 mg, yield: 11.5%) was obtained as a green solid. MS: m/z=565.2 [M+H]⁺. ¹H NMR (400 MHz, Chloroform-d₆) δ 8.60 (s, 1H), 8.14 (d, J=8.4 Hz, 1H), 8.07 (d, J=4.8 Hz, 1H), 8.02 (d, J=7.6 Hz, 2H), 7.81 (d, J=8.4 Hz, 1H), 7.51 (d, J=8.0 Hz, 1H), 7.47-7.42 (m, 4H), 7.41-7.36 (m, 1H), 7.11 (d, J=7.6 Hz, 1H), 6.84 (s, 1H), 6.66 (br s, 2H), 6.37 (dd, J=7.6, 4.8 Hz, 1H), 3.88-3.68 (m, 4H), 3.67 (s, 2H), 2.61 (t, J=4.8 Hz, 4H).

### Example 79: 5-(4-(4-(2-(2-Aminopyridin-3-yl)-3H-imidazo[4,5-b]pyridin-3-yl)benzyl)piperazine-1-carbonyl)nicotinonitrile

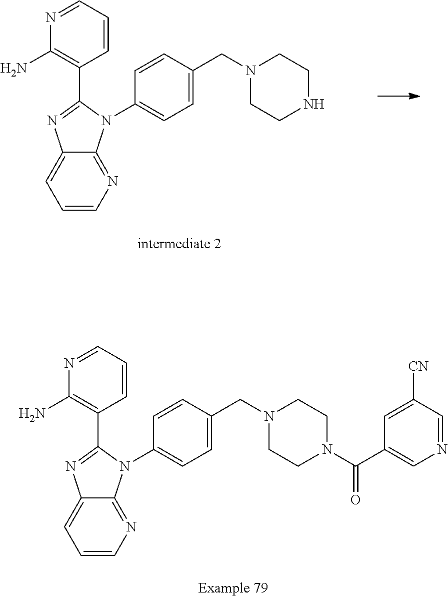 [SMILES: Nc1ncccc1-c1nc2cccnc2n1-c1ccc(CN2CCNCC2)cc1 ; N#Cc1cncc(C(=O)N2CCN(Cc3ccc(-n4c(-c5cccnc5N)nc5cccnc54)cc3)CC2)c1]

Following the general procedure of Example 23, the reaction of Intermediate 2 (200 mg, 519 μmol) with 5-cyanopyridine-3-carboxylic acid (92 mg, 623 μmol) was carried out. After purified by prep-HPLC (column: Phenomenex Luna C18 150*25 mm*10 μm; mobile phase: [water (NH₄HCO₃)-ACN]; B %: 23%-53%, 8 min), 5-(4-(4-(2-(2-aminopyridin-3-yl)-3H-imidazo[4,5-b]pyridin-3-yl)benzyl)piperazine-1-carbonyl)nicotinonitrile (Example 79, 47.6 mg, yield: 18%) was obtained as a yellow solid. MS: m/z=516.2 [M+H]⁺. ¹H NMR (400 MHz, Chloroform-d) δ 8.93 (d, J=2.0 Hz, 1H), 8.85 (d, J=2.0 Hz, 1H), 8.40 (dd, J=4.8, 1.2 Hz, 1H), 8.11 (dd, J=8.0, 1.2 Hz, 1H), 8.07-8.04 (m, 2H), 7.50 (d, J=8.0 Hz, 2H), 7.36 (d, J=8.4 Hz, 2H), 7.33-7.30 (m, J H), 7.07 (dd, J=7.6, 1.6 Hz, 1H), 6.62 (br s, 2H), 6.33 (dd, J=8.0, 4.8 Hz, 1H), 3.89-3.79 (m, 2H), 3.64 (s, 2H) 3.50-3.39 (m, 2H), 2.64-2.57 (m, 2H), 2.54-2.46 (m, 2H).

### Example 80: 6-(4-(4-(2-(2-Aminopyridin-3-yl)-3H-imidazo[4,5-b]pyridin-3-yl)benzyl)piperazine-1-carbonyl)nicotinonitrile

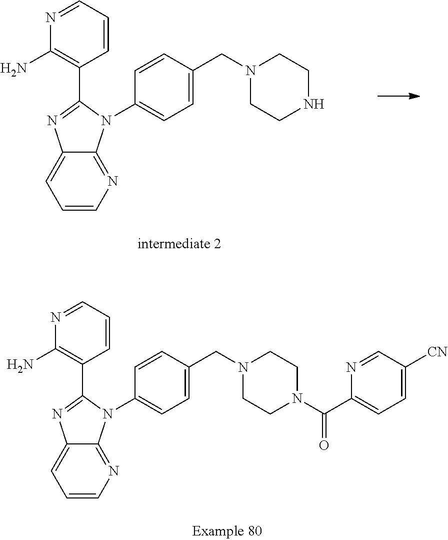 [SMILES: Nc1ncccc1-c1nc2cccnc2n1-c1ccc(CN2CCNCC2)cc1 ; N#Cc1ccc(C(=O)N2CCN(Cc3ccc(-n4c(-c5cccnc5N)nc5cccnc54)cc3)CC2)nc1]

Following the general procedure of Example 23, the reaction of Intermediate 2 (200 mg, 519 μmol) with 5-cyanopyridine-2-carboxylic acid (92 mg, 623 μmol,) was carried out. After purified by prep-HPLC (column: Phenomenex Luna C18 150*25 mm*10 μm; mobile phase: [water (NH₄HCO₃)-ACN]; B %: 23%-53%, 8 min), 6-(4-(4-(2-(2-aminopyridin-3-yl)-3H-imidazo[4,5-b]pyridin-3-yl)benzyl)piperazine-1-carbonyl)nicotinonitrile (Example 80, 41.4 mg, yield: 15%) was obtained as a yellow solid. MS: m/z=516.2 [M+H]⁺. ¹H NMR (400 MHz, Chloroform-d) 8.86 (d, J=1.6 Hz, 1H), 8.40 (dd, J=4.8, 1.2 Hz, 1H), 8.12-8.04 (m, 3H), 7.81 (d, J=7.6 Hz, 1H), 7.51 (d, J=8.0 Hz, 2H), 7.38-7.30 (m, 3H), 7.08 (dd, J=7.6, 1.6 Hz, 1H), 6.75 (br s, 2H), 6.34 (dd, J=7.6, 4.8 Hz, 1H), 3.89-3.84 (m, 2H), 3.64 (s, 2H), 3.62-3.59 (m, 2H), 2.65-2.61 (m, 2H), 2.55-2.51 (m, 2H).

### Example 81: N-(1-(4-(2-(2-Aminopyridin-3-yl)-5-phenyl-3H-imidazo[4,5-b]pyridin-3-yl)benzyl)piperidin-4-yl)-5-cyanopicolinamide

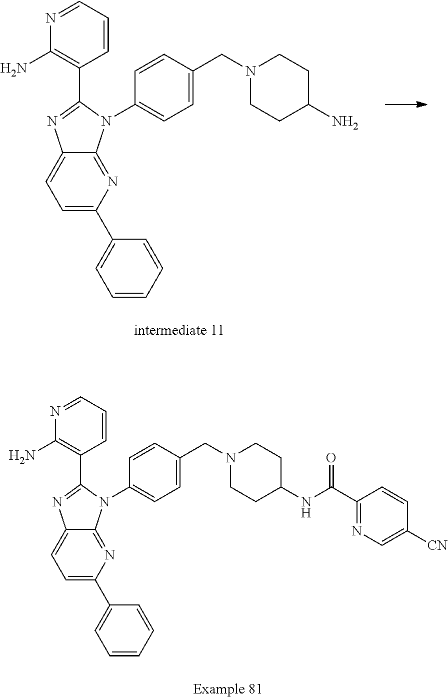 [SMILES: Nc1ncccc1-c1nc2ccc(-c3ccccc3)nc2n1-c1ccc(CN2CCC(N)CC2)cc1 ; N#Cc1ccc(C(=O)NC2CCN(Cc3ccc(-n4c(-c5cccnc5N)nc5ccc(-c6ccccc6)nc54)cc3)CC2)nc1]

Following the general procedure of Example 23, the reaction of Intermediate 11 (187 mg, 351 μmol,) with 5-cyanopicolinic acid (57.0 mg, 386 μmol) was carried out. After purified by prep-HPLC (column: Phenomenex luna C18 150-25 mm×10 Im; mobile phase: [water (FA)-ACN]; B %: 8%-38%, 9 min), N-(1-(4-(2-(2-aminopyridin-3-yl)-5-phenyl-3H-imidazo[4,5-b]pyridin-3-yl)benzyl)piperidin-4-yl)-5-cyanopicolinamide (Example 81, 51.8 mg, yield: 24%) was obtained as an off-white lyophilized powder. MS: m/z=606.4 [M+H]⁺. ¹H NMR (400 MHz, Dimethylsulfoxide-d₆) δ 9.16-9.05 (m, 1H), 8.80 (d, J=8.4 Hz, 1H), 8.51 (dd, J=8.4, 2.0 Hz, 1H), 8.27 (d, J=8.4 Hz, 1H), 8.17 (d, J=8.0 Hz, 1H), 8.07-7.95 (m, 4H), 7.51-7.43 (m, 6H), 7.42-7.36 (m, 1H), 7.16 (dd, J=7.6, 1.6 Hz, 1H), 7.03 (br s, 2H), 6.38 (dd, J=7.6, 4.8 Hz, 1H), 3.93-3.79 (m, 1H), 3.58 (s, 2H), 2.87-2.84 (m, 2H), 2.17-2.06 (m, 2H), 1.79-1.67 (m, 4H).

### Example 82: 4-(4-(4-(2-(2-Aminopyridin-3-yl)-5-phenyl-3H-imidazo[4,5-b]pyridin-3-yl)benzyl)piperazin-1-yl)picolinonitrile

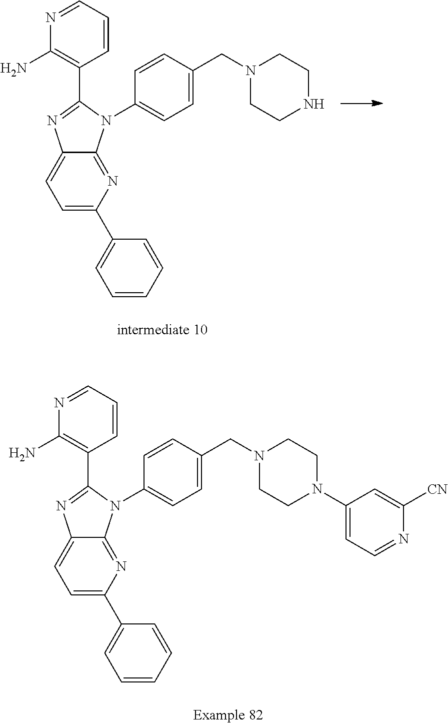 [SMILES: Nc1ncccc1-c1nc2ccc(-c3ccccc3)nc2n1-c1ccc(CN2CCNCC2)cc1 ; N#Cc1cc(N2CCN(Cc3ccc(-n4c(-c5cccnc5N)nc5ccc(-c6ccccc6)nc54)cc3)CC2)ccn1]

Following the general procedure of Example 14, the reaction of Intermediate 10 (130 mg, 281 μmol) with 4-fluoropicolinonitrile (38.0 mg, 310 μmol) was carried out. After purified by prep-HPLC (column: Welch Xtimate C18 150*25 mm*5 μm; mobile phase: [water (NH₄HCO₃)-ACN]; B %: 37%-67%, 2 min), 4-(4-(4-(2-(2-aminopyridin-3-yl)-5-phenyl-3H-imidazo[4,5-b]pyridin-3-yl)benzyl)piperazin-1-yl)picolinonitrile (Example 82, 47.0 mg, yield: 30%) was obtained as a light-yellow lyophilized powder. MS: m/z=564.4 [M+H]⁺. ¹H NMR (400 MHz, Dimethylsulfoxide-d₆) δ 8.27 (d, J=8.4 Hz, 1H), 8.23 (d, J=6.0 Hz, 1H), 8.07-7.96 (m, 4H), 7.56-7.44 (m, 7H), 7.43-7.37 (m, 1H), 7.15 (dd, J=7.6, 1.6 Hz, 1H), 7.07 (dd, J=6.4, 2.8 Hz, 1H), 7.03 (br s, 2H), 6.36 (dd, J=7.6, 4.8 Hz, 1H), 3.64 (s, 2H), 3.50-3.43 (m, 4H), 2.55-2.51 (m, 4H).

### Example 83: 6-(4-(4-(2-(2-Aminopyridin-3-yl)-5-phenyl-3H-imidazo[4,5-b]pyridin-3-yl)benzyl)piperazin-1-yl)picolinonitrile

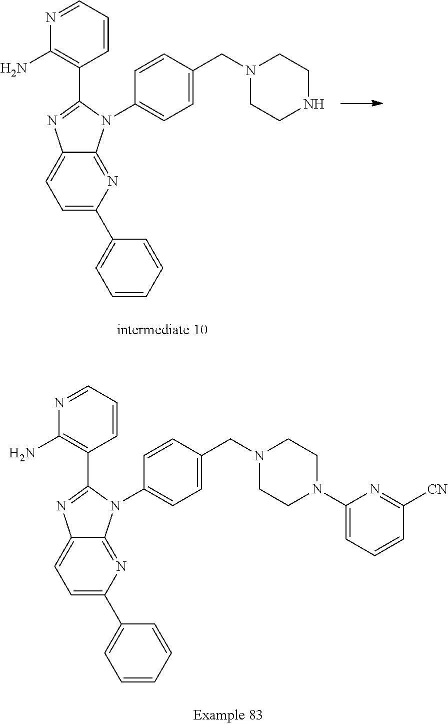 [SMILES: Nc1ncccc1-c1nc2ccc(-c3ccccc3)nc2n1-c1ccc(CN2CCNCC2)cc1 ; N#Cc1cccc(N2CCN(Cc3ccc(-n4c(-c5cccnc5N)nc5ccc(-c6ccccc6)nc54)cc3)CC2)n1]

Following the general procedure Example 14, the reaction of Intermediate 10 (130 mg, 281 μmol) with 6-fluoropicolinonitrile (35.0 mg, 287 μmol) was carried out. After purified by prep-HPLC (column: Welch Xtimate C18 150×25 mm×5 μm; mobile phase: [water (NH₄HCO₃)-ACN]; B %: 37%-67%, 2 min), 6-(4-(4-(2-(2-aminopyridin-3-yl)-5-phenyl-3H-imidazo[4,5-b]pyridin-3-yl)benzyl)piperazin-1-yl)picolinonitrile (Example 83, 34.3 mg, yield: 22%) was obtained as a light-yellow lyophilized powder. MS: m/z=564.3 [M+H]⁺. ¹H NMR (400 MHz, Dimethylsulfoxide-d₆) δ 8.27 (d, J=8.4 Hz, 1H), 8.09-7.96 (m, 4H), 7.70 (dd, J=8.8, 7.6 Hz, 1H), 7.54-7.50 (m, 2H), 7.50-7.44 (m, 4H), 7.43-7.37 (m, 1H), 7.24-7.13 (m, 3H), 7.03 (br s, 2H), 6.37 (dd, J=8.0, 4.8 Hz, 1H), 3.64 (s, 2H), 3.62-3.56 (m, 4H), 2.54-2.51 (m, 4H).

### Example 84: 5-((1-(4-(2-(2-Aminopyridin-3-yl)-5-phenyl-3H-imidazo[4,5-b]pyridin-3-yl)benzyl)piperidin-4-yl)amino)picolinonitrile

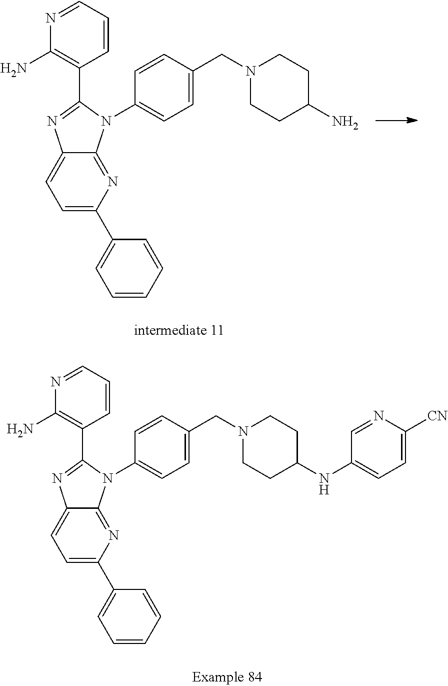 [SMILES: Nc1ncccc1-c1nc2ccc(-c3ccccc3)nc2n1-c1ccc(CN2CCC(N)CC2)cc1 ; N#Cc1ccc(NC2CCN(Cc3ccc(-n4c(-c5cccnc5N)nc5ccc(-c6ccccc6)nc54)cc3)CC2)cn1]

To a solution of Intermediate 11 (150 mg, 315 μmol) in DMF (2 mL) was added 5-fluoropyridine-2-carbonitrile (46.2 mg, 378 μmol) and DIEA (204 mg, 1.58 mmol). The mixture was stirred at 100° C. for 16 hr. The mixture was concentrated under reduced pressure to give a residue. The mixture was purified by prep-HPLC (column: Waters Xbridge 150×25 mm×5 μm; mobile phase: [water (NH₄HCO₃)-ACN]; B %: 35%-75%, 8 min). to give 5-((1-(4-(2-(2-aminopyridin-3-yl)-5-phenyl-3H-imidazo[4,5-b]pyridin-3-yl)benzyl)piperidin-4-yl)amino)picolinonitrile (Example 84, 24.7 mg, yield: 14%) as a light-yellow lyophilized powder. MS: m/z=578.2 [M+H]⁺. ¹H NMR (400 MHz, Chloroform-d) 58.15 (d, J=8.4 Hz, 1H), 8.07 (dd, J=4.8, 1.2 Hz, 1H), 8.04-8.01 (m, 3H), 7.82 (d, J=8.4 Hz, 1H), 7.57-7.51 (m, 2H), 7.48-7.35 (m, 7H), 7.10 (dd, J=7.8, 1.2 Hz, 1H), 6.82 (dd, J=8.4, 2.8 Hz, 1H), 6.64 (br s, 1H), 6.37 (dd, J=7.6, 4.8 Hz, 1H), 4.29 (br s, 1H), 3.71 (s, 2H), 3.49-3.35 (m, 1H), 3.09-2.93 (m, 2H), 2.33-2.30 (m, 2H), 2.17-2.05 (m, 2H), 1.68-1.67 (m, 2H).

### Example 85: 4-((1-(4-(2-(2-Aminopyridin-3-yl)-5-phenyl-3H-imidazo[4,5-b]pyridin-3-yl)benzyl)piperidin-4-yl)amino)picolinonitrile

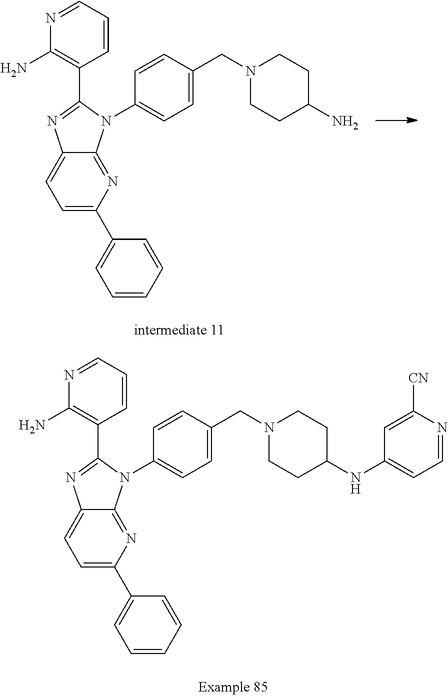 [SMILES: Nc1ncccc1-c1nc2ccc(-c3ccccc3)nc2n1-c1ccc(CN2CCC(N)CC2)cc1 ; N#Cc1cc(NC2CCN(Cc3ccc(-n4c(-c5cccnc5N)nc5ccc(-c6ccccc6)nc54)cc3)CC2)ccn1]

Following the general procedure of Example 84, the reaction of Intermediate 11 (150 mg, 315 μmol) with 4-fluoropyridine-2-carbonitrile (46.2 mg, 378 μmol) was carried out in DMSO (3 mL). After purified by prep-HPLC (column: Waters Xbridge BEH C18 100×30 mm×10 μm; mobile phase: [water (NH₄HCO₃)-ACN]; B %: 44%-74%, 2 min), 4-((1-(4-(2-(2-aminopyridin-3-yl)-5-phenyl-3H-imidazo[4,5-b]pyridin-3-yl)benzyl)piperidin-4-yl)amino)picolinonitrile (Example 85, 31.7 mg, yield: 17%) was obtained as a light-yellow lyophilized powder. MS: m/z=578.2 [M+H]⁺. ¹H NMR (400 MHz, Chloroform-d) δ 8.22 (d, J=5.6 Hz, 1H), 8.15 (d, J=8.4 Hz, 1H), 8.07 (dd, J=4.8, 1.6 Hz, 1H), 8.04-8.01 (m, 2H), 7.82 (d, J=8.4 Hz, 1H), 7.58-7.53 (m, 2H), 7.47-7.43 (m, 4H), 7.41-7.36 (m, 1H), 7.11 (dd, J=7.6, 1.6 Hz, 1H), 6.83 (d, J=2.4 Hz, 1H), 6.68 (br s, 2H), 6.57 (dd, J=5.6, 2.4 Hz, 1H), 6.37 (dd, J=7.6, 4.8 Hz, 1H), 4.52 (br s, 1H), 3.75 (s, 2H), 3.49-3.38 (m, 1H), 3.10-3.00 (m, 2H), 2.43-2.32 (m, 2H), 2.14-2.07 (m, 2H), 1.76-1.75 (m, 2H)

### Example 86: 6-((1-(4-(2-(2-Aminopyridin-3-yl)-5-phenyl-3H-imidazo[4,5-b]pyridin-3-yl)benzyl)piperidin-4-yl)amino)picolinonitrile

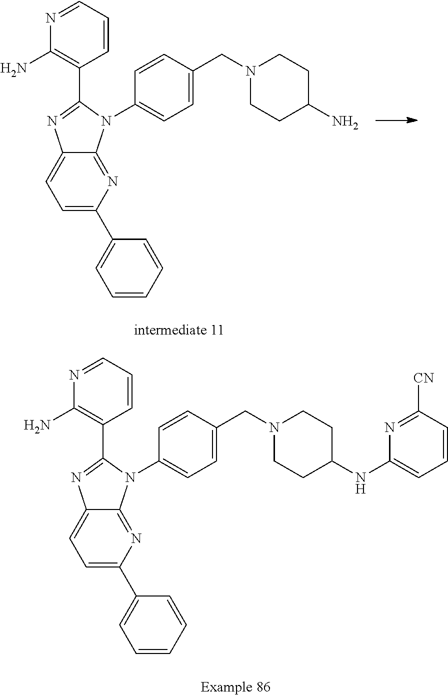 [SMILES: Nc1ncccc1-c1nc2ccc(-c3ccccc3)nc2n1-c1ccc(CN2CCC(N)CC2)cc1 ; N#Cc1cccc(NC2CCN(Cc3ccc(-n4c(-c5cccnc5N)nc5ccc(-c6ccccc6)nc54)cc3)CC2)n1]

Following the general procedure of Example 84, the reaction of Intermediate 11 (150 mg, 315 μmol) with 6-fluoropyridine-2-carbonitrile (46.2 mg, 378 μmol) was carried out in DMSO (3 mL). After purified by prep-HPLC (column: Phenomenex C18 150×25 mm×10 μm; mobile phase: [water (NH₄HCO₃)-ACN]; B %: 45%-75%, 8 min), 6-((1-(4-(2-(2-aminopyridin-3-yl)-5-phenyl-3H-imidazo[4,5-b]pyridin-3-yl)benzyl)piperidin-4-yl)amino)picolinonitrile (Example 86, 15.6 mg, yield: 8.6%) was obtained as a light-yellow lyophilized powder. MS: m/z=578.2 [M+H]⁺. ¹H NMR (400 MHz, Chloroform-d) 58.16 (d, J=8.4 Hz, 1H), 8.08 (dd, J=4.8, 1.2 Hz, 1H), 8.02 (d, J=7.2 Hz, 2H), 7.86-7.82 (m, 2H), 7.58-7.53 (m, 2H), 7.49-7.37 (m, 5H), 7.10 (dd, J=7.6, 1.2 Hz, 1H), 7.02-6.96 (m, 1H), 6.74 (br s, 2H), 6.62 (d, J=8.4 Hz, 1H), 6.41 (dd, J=7.6, 4.8 Hz, 1H), 4.90-4.88 (m, 1H), 4.26-4.06 (m, 3H), 3.51-3.49 (m, 2H), 2.89-2.70 (m, 2H), 2.28-2.27 (m, 2H), 1.37-1.23 (m, 2H).

### Example 87: 2-((1-(4-(2-(2-Aminopyridin-3-yl)-5-phenyl-3H-imidazo[4,5-b]pyridin-3-yl)benzyl)piperidin-4-yl)amino)isonicotinonitrile

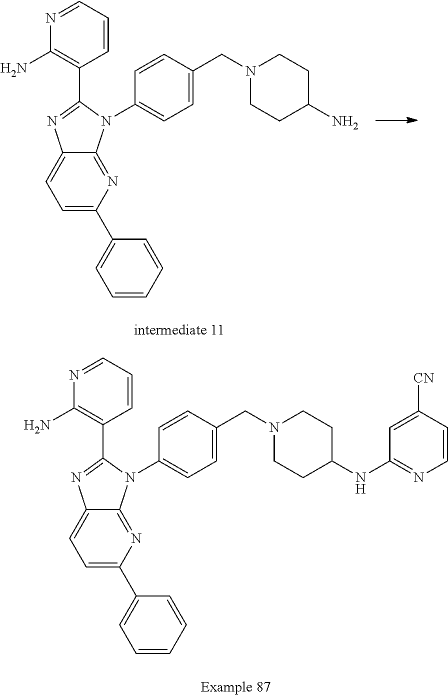 [SMILES: Nc1ncccc1-c1nc2ccc(-c3ccccc3)nc2n1-c1ccc(CN2CCC(N)CC2)cc1 ; N#Cc1ccnc(NC2CCN(Cc3ccc(-n4c(-c5cccnc5N)nc5ccc(-c6ccccc6)nc54)cc3)CC2)c1]

Following the general procedure of Example 84, the reaction of Intermediate 11 (208 mg, 420 μmol) with 2-chloropyridine-4-carbonitrile (58.3 mg, 420 μmol) was carried out under microwave at 160° C. for 2 hr. After purified by prep-HPLC (column: Phenomenex C18 150×25 mm×10 μm; mobile phase: [water (NH₄HCO₃)-ACN]; B %: 51%-81%, 8 min), 2-((1-(4-(2-(2-aminopyridin-3-yl)-5-phenyl-3H-imidazo[4,5-b]pyridin-3-yl)benzyl)piperidin-4-yl)amino)isonicotinonitrile (Example 87, 5.2 mg, yield: 2.1%) was obtained as a light-yellow lyophilized powder. MS: m/z=578.3 [M+H]⁺. ¹H NMR (400 MHz, Dimethylsulfoxide-d₆) δ 8.27 (d, J=8.0 Hz, 1H), 8.15 (d, J=5.2 Hz, 1H), 8.03 (d, J=7.6 Hz, 2H), 8.01-7.97 (m, 2H), 7.52-7.42 (m, 7H), 7.41-7.37 (m, 1H), 7.18-7.13 (m, 1H), 7.03 (br s, 2H), 6.80 (s, 1H), 6.78-6.72 (m, 1H), 6.38 (dd, J=7.6, 4.8 Hz, 1H), 3.79-3.65 (m, 1H), 3.58 (s, 2H), 2.88-2.80 (m, 2H), 2.16-2.10 (m, 2H), 1.94-1.87 (m, 2H), 1.52-1.42 (m, 2H).

### Example 88: 4-((1-(4-(2-(2-Aminopyridin-3-yl)-5-phenyl-3H-imidazo[4,5-b]pyridin-3-yl)benzyl)piperidin-4-yl)amino)pyrimidine-2-carbonitrile

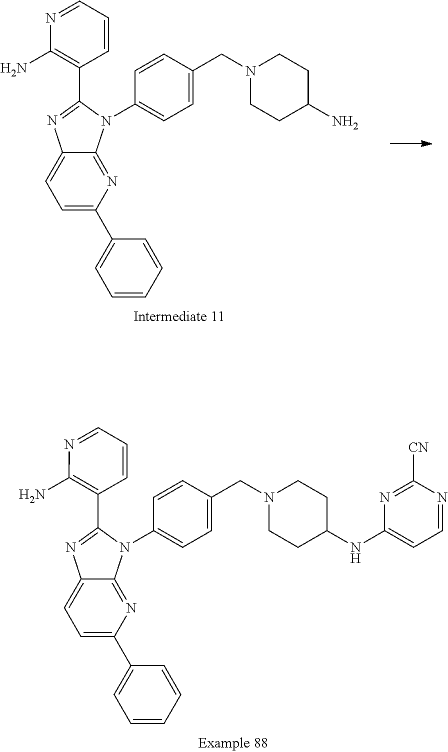 [SMILES: Nc1ncccc1-c1nc2ccc(-c3ccccc3)nc2n1-c1ccc(CN2CCC(N)CC2)cc1 ; N#Cc1nccc(NC2CCN(Cc3ccc(-n4c(-c5cccnc5N)nc5ccc(-c6ccccc6)nc54)cc3)CC2)n1]

Following the general procedure of Example 84, the reaction of Intermediate 11 (1 g, 2.0 mmol) with 4-chloropyrimidine-2-carbonitrile (278 mg, 2.0 mmol) was carried out under microwave at 130° C. for 0.5 hr. After purified by silica gel flash chromatography (Eluent of 0-100% EtOAc in petroleum ether and 8% MeOH in CH₂Cl₂), 4-((1-(4-(2-(2-Aminopyridin-3-yl)-5-phenyl-3H-imidazo[4,5-b]pyridin-3-yl)benzyl)piperidin-4-yl)amino)pyrimidine-2-carbonitrile (Example 88, 601 mg, yield: 52%) was obtained as a light-yellow solid. MS: m/z=579.4 [M+H]⁺. ¹H NMR (400 MHz, Dimethylsulfoxide-d₆) δ 8.27 (d, J=8.4 Hz, 1H), 8.10-7.97 (m, 6H), 7.51-7.43 (m, 6H), 7.41-7.37 (m, 1H), 7.16 (dd, J=7.6, 1.6 Hz, 1H), 7.03 (br s, 2H), 6.67 (d, J=5.6 Hz, 1H), 6.38 (dd, J=7.6, 4.8 Hz, 1H), 3.84-3.79 (m, 1H), 3.59 (s, 2H), 2.87-2.80 (m, 2H), 2.15-2.13 (m, 2H), 1.90-1.89 (m, 2H), 1.55-1.44 (m, 2H).

### Example 89: 6-((1-(4-(2-(2-Aminopyridin-3-yl)-5-phenyl-3H-imidazo[4,5-b]pyridin-3-yl)benzyl)piperidin-4-yl)amino)pyrazine-2-carbonitrile

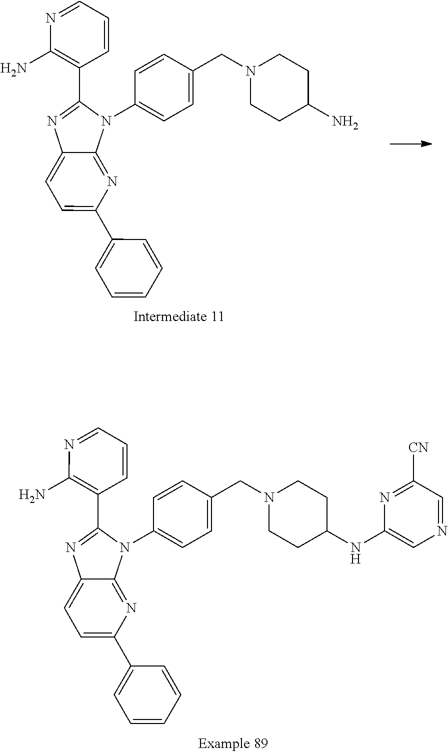 [SMILES: Nc1ncccc1-c1nc2ccc(-c3ccccc3)nc2n1-c1ccc(CN2CCC(N)CC2)cc1 ; N#Cc1cncc(NC2CCN(Cc3ccc(-n4c(-c5cccnc5N)nc5ccc(-c6ccccc6)nc54)cc3)CC2)n1]

Following the general procedure of Example 84, the reaction of Intermediate 11 (150 mg, 315 μmol) with 6-chloropyrazine-2-carbonitrile (52.8 mg, 378 μmol) was carried out under microwave at 130° C. for 0.5 hr. After purified by prep-HPLC (column: Waters xbridge 150×25 mm×10 μm; mobile phase: [water (NH₄HCO₃)-ACN]; B %: 45%-75%, 9 min), 6-((1-(4-(2-(2-Aminopyridin-3-yl)-5-phenyl-3H-imidazo[4,5-b]pyridin-3-yl)benzyl)piperidin-4-yl)amino)pyrazine-2-carbonitrile (Example 89, 20.1 mg, yield: 11%) was obtained as a light-yellow lyophilized powder. MS: m/z=579.1 [M+H]⁺. ¹H NMR (400 MHz, Chloroform-d) δ 8.15 (d, J=8.4 Hz, 1H), 8.11-8.05 (m, 3H), 8.04-8.00 (m, 2H), 7.82 (d, J=8.4 Hz, 1H), 7.66-7.59 (m, 2H), 7.49-7.43 (m, 4H), 7.41-7.37 (m, 1H), 7.10 (dd, J=7.6, 1.6 Hz, 1H), 6.63 (br s, 2H), 6.38 (dd, J=7.6, 4.8 Hz, 1H), 5.59-4.63 (m, 1H), 4.03-3.80 (m, 3H), 3.27-3.02 (m, 2H), 2.59-2.40 (m, 2H), 2.20-2.13 (m, 2H), 1.93-1.84 (m, 2H).

### Example 90: 2-((1-(4-(2-(2-Aminopyridin-3-yl)-5-phenyl-3H-imidazo[4,5-b]pyridin-3-yl)benzyl)piperidin-4-yl)amino)pyrimidine-4-carbonitrile

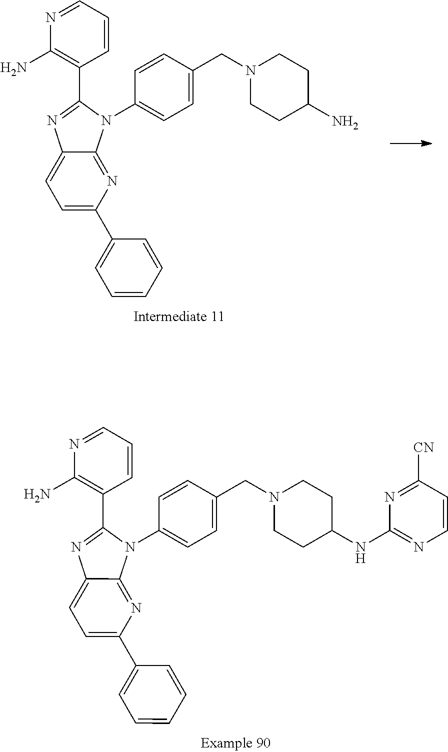 [SMILES: Nc1ncccc1-c1nc2ccc(-c3ccccc3)nc2n1-c1ccc(CN2CCC(N)CC2)cc1 ; N#Cc1ccnc(NC2CCN(Cc3ccc(-n4c(-c5cccnc5N)nc5ccc(-c6ccccc6)nc54)cc3)CC2)n1]

Following the general procedure of Example 84, the reaction of Intermediate 11 (150 mg, 315 μmol) with 2-chloropyrimidine-4-carbonitrile (52.8 mg, 378 μmol) was carried out under microwave at 130° C. for 0.5 hr. After purified by prep-HPLC (column: Waters xbridge 150×25 mm×10 μm; mobile phase: [water (NH₄HCO₃)-ACN]; B %: 45%-75%, 9 min), 2-((1-(4-(2-(2-aminopyridin-3-yl)-5-phenyl-3H-imidazo[4,5-b]pyridin-3-yl)benzyl)piperidin-4-yl)amino)pyrimidine-4-carbonitrile (Example 90, 30.9 mg, yield: 17%) was obtained as a light gray Lyophilized powder. MS: m/z=579.1 [M+H]⁺. ¹H NMR (400 MHz, Chloroformn-d) δ 8.44 (d, J=2.4 Hz, 1H), 8.14 (d, J=8.4 Hz, 1H), 8.08 (dd, J=4.8, 1.6 Hz, 1H), 8.03 (d, J=7.2 Hz, 2H), 7.82 (d, J=8.4 Hz, 1H), 7.57 (d, J=7.6 Hz, 2H), 7.49-7.42 (m, 4H), 7.41-7.35 (m, 1H), 7.11 (dd, J=7.6, 1.2 Hz, 1H), 6.83 (d, J=4.8 Hz, 1H), 6.63 (br s, 2H), 6.38 (dd, J=7.6, 4.8 Hz, 1H), 5.41 (br s, 1H), 4.00-3.90 (m, 1H), 3.74 (s, 2H), 3.18-2.90 (m, 2H), 2.39-2.37 (m, 2H), 2.14-2.11 (m, 2H), 1.79-1.78 (m, 2H).

### Example 91: 6-((1-(4-(2-(2-Aminopyridin-3-yl)-5-phenyl-3H-imidazo[4,5-b]pyridin-3-yl)benzyl)piperidin-4-yl)amino)pyrimidine-4-carbonitrile

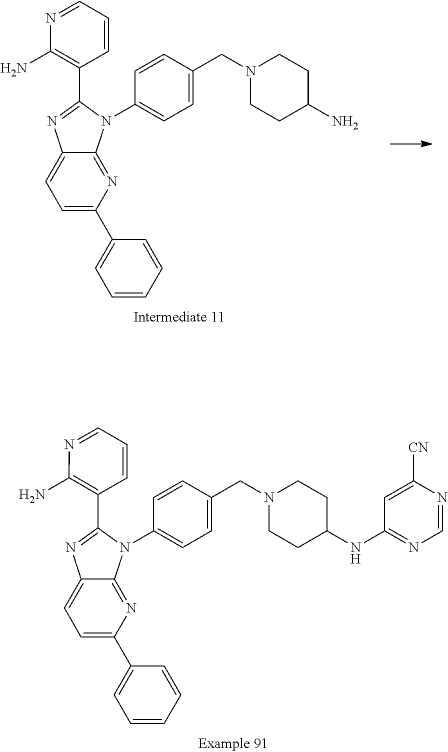 [SMILES: Nc1ncccc1-c1nc2ccc(-c3ccccc3)nc2n1-c1ccc(CN2CCC(N)CC2)cc1 ; N#Cc1cc(NC2CCN(Cc3ccc(-n4c(-c5cccnc5N)nc5ccc(-c6ccccc6)nc54)cc3)CC2)ncn1]

Following the general procedure of Example 84, the reaction of Intermediate 11 (150 mg, 315 μmol) with 6-chloropyrimidine-4-carbonitrile (52.8 mg, 378 μmol) was carried out under microwave at 130° C. for 0.5 hr. After purified by silica gel flash chromatography (Eluent of 0˜20% MeOH in CH₂Cl₂), 6-((1-(4-(2-(2-Aminopyridin-3-yl)-5-phenyl-3H-imidazo[4,5-b]pyridin-3-yl)benzyl)piperidin-4-yl)amino)pyrimidine-4-carbonitrile (Example 91, 31.3 mg, yield: 17%) was obtained as a yellow-green lyophilized powder. MS: m/z=579.2 [M+H]⁺. ¹H NMR (400 MHz, Dimethylsulfoxide-d₆) δ 8.50 (s, 1H), 8.27 (d, J=8.4 Hz, 1H), 8.09-7.97 (m, 5H), 7.50-7.44 (m, 6H), 7.42-7.36 (m, 1H), 7.16 (dd, J=7.6, 1.6 Hz, 1H), 7.02 (br s, 2H), 6.93 (s, 1H), 6.38 (dd, J=7.6, 4.8 Hz, 1H), 3.94-3.80 (m, 1H), 3.59 (s, 2H), 2.87-2.80 (m, 2H), 2.17-2.07 (m, 2H), 1.95-1.83 (m, 2H), 1.55-1.47 (m, 2H).

### Example 92: 4-((1-(4-(2-(2-Aminopyridin-3-yl)-3H-imidazo[4,5-b]pyridin-3-yl)benzyl)piperidin-4-yl)amino)pyrimidine-2-carbonitrile

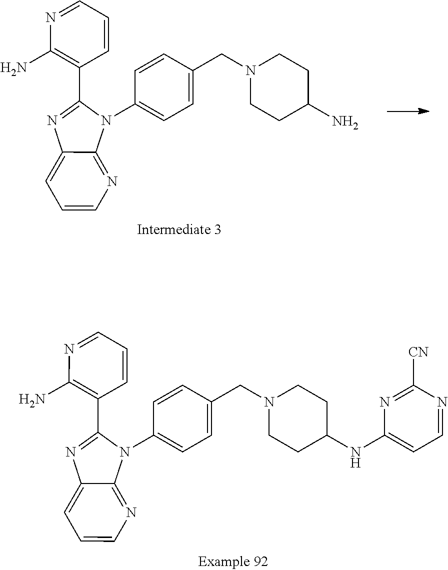 [SMILES: Nc1ncccc1-c1nc2cccnc2n1-c1ccc(CN2CCC(N)CC2)cc1 ; N#Cc1nccc(NC2CCN(Cc3ccc(-n4c(-c5cccnc5N)nc5cccnc54)cc3)CC2)n1]

Following the general procedure of Example 84, the reaction of Intermediate 3 (150 mg, 375 μmol) with 6-chloropyrimidine-4-carbonitrile (52.4 mg, 375 μmol) was carried out under microwave at 130° C. for 0.5 hr. After purified by silica gel flash chromatography (Eluent of 0-13% EtOAc in petroleum ether) and then prep-TLC (CH₂Cl₂:MeOH=10:1), 4-((1-(4-(2-(2-aminopyridin-3-yl)-3H-imidazo[4,5-b]pyridin-3-yl)benzyl)piperidin-4-yl)amino)pyrimidine-2-carbonitrile (Example 92, 24.7 mg, yield: 13%) was obtained as a light-gray solid. MS: m/z=503.3 [M+H]⁺. ¹H NMR (400 MHz, Dimethylsulfoxide-d₆) δ 8.33 (dd, J=4.6, 1.2 Hz, 1H), 8.20 (dd, J=8.0, 1.2 Hz, 1H), 8.11-8.03 (m, 2H), 7.99 (dd, J=4.8, 1.6 Hz, 1H), 7.45 (d, J=8.4 Hz, 2H), 7.41-7.36 (m, 3H), 7.16 (dd, J=7.6, 1.6 Hz, 1H), 7.01 (br s, 2H), 6.67 (d, J=5.6 Hz, 1H), 6.37 (dd, J=7.6, 4.8 Hz, 1H), 3.84-3.80 (m, 1H), 3.56 (s, 2H), 2.81 (d, J=11.2 Hz, 2H), 2.18-2.11 (m, 2H), 1.89-1.87 (m, 2H), 1.53-1.44 (m, 2H).

### Example 93: 5-((1-(4-(2-(2-Aminopyridin-3-yl)-5-phenyl-3H-imidazo[4,5-b]pyridin-3-yl)benzyl)piperidin-4-yl)amino)nicotinonitrile

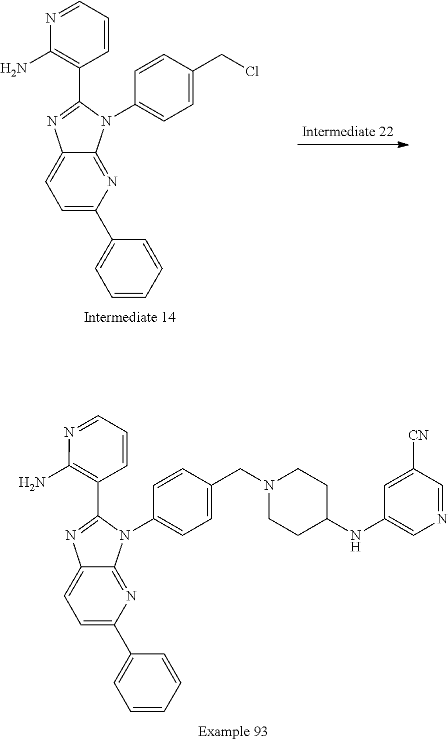 [SMILES: Nc1ncccc1-c1nc2ccc(-c3ccccc3)nc2n1-c1ccc(CCl)cc1 ; N#Cc1cncc(NC2CCN(Cc3ccc(-n4c(-c5cccnc5N)nc5ccc(-c6ccccc6)nc54)cc3)CC2)c1]

To a solution of Intermediate 14 (120 mg, 291 μmol), Intermediate 22 (64.8 mg, 320 μmol) in DMF (1 mL) were added NaI (4.37 mg, 29.1 μmol) and K₂CO₃ (80.5 mg, 582 μmol). The mixture was stirred at 80° C. for 2 hr. The reaction mixture was diluted with H₂O (5 mL) and then filtered and concentrated under reduced pressure to give a residue. The residue was purified by prep-TLC (CH₂Cl₂:MeOH=10:1), 5-((1-(4-(2-(2-aminopyridin-3-yl)-5-phenyl-3H-imidazo[4,5-b]pyridin-3-yl)benzyl)piperidin-4-yl)amino)nicotinonitrile (Example 93, 13.7 mg, yield: 8.2%) was obtained as a light-yellow powder. MS: m/z=578.4 [M+H]⁺. ¹H NMR (400 MHz, Dimethylsulfoxide-d₆) δ 8.27 (d, J=8.4 Hz, 1H), 8.22 (d, J=2.8 Hz, 1H), 8.08-7.97 (m, 5H), 7.51-7.44 (m, 6H), 7.43-7.36 (m, 1H), 7.32 (s, 1H), 7.16 (dd, J=7.6, 2.0 Hz, 1H), 7.02 (br s, 2H), 6.42-6.32 (m, 2H), 3.59 (s, 2H), 3.30-3.10 (m, 1H), 2.95-2.75 (m, 2H), 2.18 (t, J=9.6 Hz, 2H), 1.92-1.85 (m, 2H), 1.46-1.38 (m, 2H).

### Example 94: 5-(4-(4-(2-(2-Aminopyridin-3-yl)-5-phenyl-3H-imidazo[4,5-b]pyridin-3-yl)benzyl)piperazin-1-yl)picolinonitrile

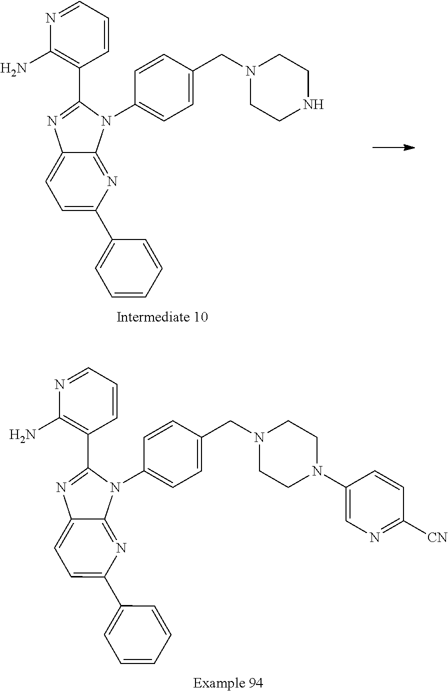 [SMILES: Nc1ncccc1-c1nc2ccc(-c3ccccc3)nc2n1-c1ccc(CN2CCNCC2)cc1 ; N#Cc1ccc(N2CCN(Cc3ccc(-n4c(-c5cccnc5N)nc5ccc(-c6ccccc6)nc54)cc3)CC2)cn1]

Following the general procedure of Example 14, the reaction of Intermediate 10 (200 mg, 433 μmol) with 5-fluoropicolinonitrile (63.4 mg, 520 μmol) was carried out. After purified by prep-HPLC (column: Waters xbridge 150*25 mm 10 um; mobile phase: [water (NH₄HCO₃)-ACN]; B %: 44%-74%, 8 min), 5-(4-(4-(2-(2-Aminopyridin-3-yl)-5-phenyl-3H-imidazo[4,5-b]pyridin-3-yl)benzyl)piperazin-1-yl)picolinonitrile (Example 94, 49.5 mg, yield: 20%) was obtained as an orange solid. MS: m/z=564.0 [M+H]⁺. ¹H NMR (400 MHz, Dimethylsulfoxide-d₆) δ 8.44 (d, J=2.8 Hz, 1H), 8.28 (d, J=8.4 Hz, 1H), 8.06-7.98 (m, 4H), 7.75 (d, J=9.2 Hz, JH), 7.530 (d, J=8.4 Hz, 2H), 7.50-7.44 (m, 4H), 7.43-7.35 (m, 2H), 7.16 (dd, J=7.6, 1.6 Hz, 1H), 7.04 (br s, 2H), 6.38 (dd, J=7.6, 4.8 Hz, 1H), 3.66 (s, 2H), 3.46 (t, J=4.8 Hz, 4H), 2.56 (t, J=4.8 Hz, 4H).

### Example 95: 5-((4-(2-(2-Aminopyridin-3-yl)-3H-imidazo[4,5-b]pyridin-3-yl)benzyl)amino)pyrimidine-2-carbonitrile

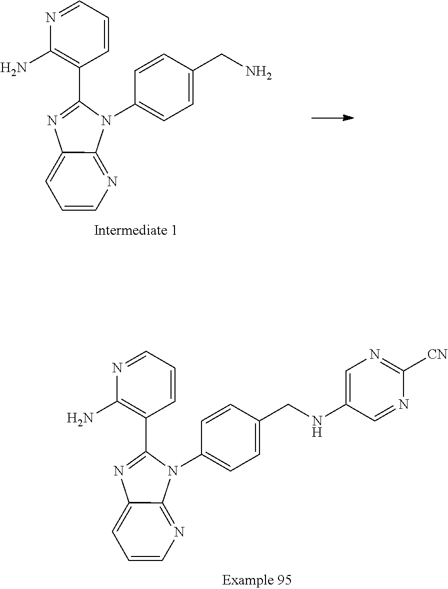 [SMILES: NCc1ccc(-n2c(-c3cccnc3N)nc3cccnc32)cc1 ; N#Cc1ncc(NCc2ccc(-n3c(-c4cccnc4N)nc4cccnc43)cc2)cn1]

To a solution of Intermediate 1 (200 mg, 0.632 mmol) and 5-fluoropyrimidine-2-carbonitrile (77.8 mg, 0.632 mmol) in DMF (4 mL) was added DIEA (245 mg, 1.90 mmol). The mixture was stirred at 100° C. for 12 hr. The reaction mixture was filtered. Then the filtrate was purified by prep-HPLC (column: Waters xbridge 150*25 mm 10 μm; mobile phase: [water (NH₄HCO₃)-ACN]; B %: 17%-47%, 10 min), 5-((4-(2-(2-aminopyridin-3-yl)-3H-imidazo[4,5-b]pyridin-3-yl)benzyl)amino)pyrimidine-2-carbonitrile (Example 95, 52.8 mg, yield: 20%) was obtained as a white solid. MS: m/z=420.1 [M+H]⁺. ¹H NMR (400 MHz, Dimethylsulfoxide-d₆) δ 8.31 (dd, J=4.8, 1.6 Hz, 1H), 8.26 (s, 2H), 8.20 (dd, J=8.0, 1.2 Hz, 1H), 7.99 (m, dd, J=8.8, 2.0 Hz, 1H), 7.85 (dd, J=6.0, 6.0 Hz, 1H), 7.52 (d, J=8.4 Hz, 2H), 7.45-7.33 (m, 3H), 7.17 (dd, J=7.6, 1.6 Hz, 1H), 6.97 (br s, 2H), 6.36 (dd, J=7.6, 4.8 Hz, 1H), 4.55 (d, J=6.0 Hz, 2H).

### Example 96: 5-((4-(2-(2-Aminopyridin-3-yl)-5-phenyl-3H-imidazo[4,5-b]pyridin-3-yl)benzyl)amino)pyrimidine-2-carbonitrile

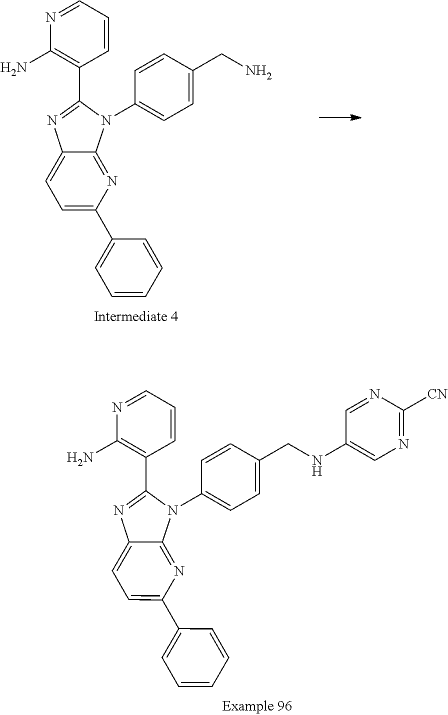 [SMILES: NCc1ccc(-n2c(-c3cccnc3N)nc3ccc(-c4ccccc4)nc32)cc1 ; N#Cc1ncc(NCc2ccc(-n3c(-c4cccnc4N)nc4ccc(-c5ccccc5)nc43)cc2)cn1]

Following the general procedure of Example 95, the reaction of Intermediate 4 (200 mg, 509 μmol) with 5-fluoropyrimidine-2-carbonitrile (63 mg, 509 μmol) was carried out. After purified by prep-HPLC (column: Waters xbridge 150*25 mm 10 μm; mobile phase: [water (NH₄HCO₃)-ACN]; B %: 35%-65%, 10 min), 5-((4-(2-(2-aminopyridin-3-yl)-5-phenyl-3H-imidazo[4,5-b]pyridin-3-yl)benzyl)amino)pyrimidine-2-carbonitrile (Example 96, 55 mg, yield: 22%) was obtained as an off-white lyophilized powder. MS: m/z=496.3 [M+H]⁺. ¹H NMR (400 MHz, Dimethylsulfoxide-d₆) δ 8.36-8.20 (m, 3H), 8.09-7.95 (m, 4H), 7.87-7.77 (m, 1H), 7.60-7.36 (m, 7H), 7.21-7.14 (m, 1H), 6.99 (br s, 2H), 6.44-6.31 (m, 1H), 4.57 (d, J=5.2 Hz, 2H).

### Example 97: 5-(4-(4-(2-(2-aminopyridin-3-yl)-5-phenyl-3H-imidazo[4,5-b]pyridin-3-yl)benzyl)piperazin-1-yl)pyrimidine-2-carbonitrile

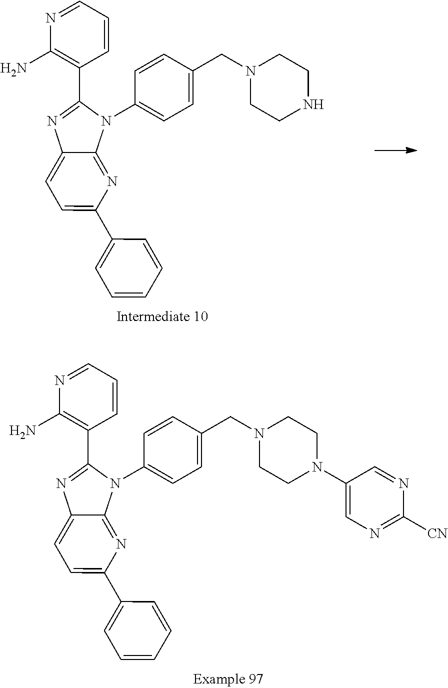 [SMILES: Nc1ncccc1-c1nc2ccc(-c3ccccc3)nc2n1-c1ccc(CN2CCNCC2)cc1 ; N#Cc1ncc(N2CCN(Cc3ccc(-n4c(-c5cccnc5N)nc5ccc(-c6ccccc6)nc54)cc3)CC2)cn1]

Following the general procedure of Example 95, the reaction of Intermediate 10 (200 mg, 433 μmol) with 5-fluoropyrimidine-2-carbonitrile (53.3 mg, 433 μmol) was carried out. After purified by prep-HPLC (column: Waters xbridge 150*25 mm 10 μm; mobile phase: [water (NH₄HCO₃)-ACN]; B %: 42%-72%, 10 min), 5-(4-(4-(2-(2-aminopyridin-3-yl)-5-phenyl-3H-imidazo[4,5-b]pyridin-3-yl)benzyl)piperazin-1-yl)pyrimidine-2-carbonitrile (Example 97, 18 mg, yield: 7.4%) was obtained as a yellow solid. MS: m/z=565.3 [M+H]⁺. ¹H NMR (400 MHz, Dimethylsulfoxide-d₆) δ 8.58 (s, 2H), 8.27 (d, J=8.4 Hz, 1H), 8.10-7.95 (m, 4H), 7.56-7.44 (m, 6H), 7.43-7.34 (m, 1H), 7.15 (d, J=6.0 Hz, 1H), 7.03 (br s, 2H), 6.37 (dd, J=7.6, 4.8 Hz, 1H), 3.65 (s, 2H), 3.58-3.46 (m, 4H), 2.63-2.39 (m, 4H).

### Example 98: N-(4-(2-(2-Aminopyridin-3-yl)-3H-imidazo[4,5-b]pyridin-3-yl)benzyl)-2-cyanonicotinamide

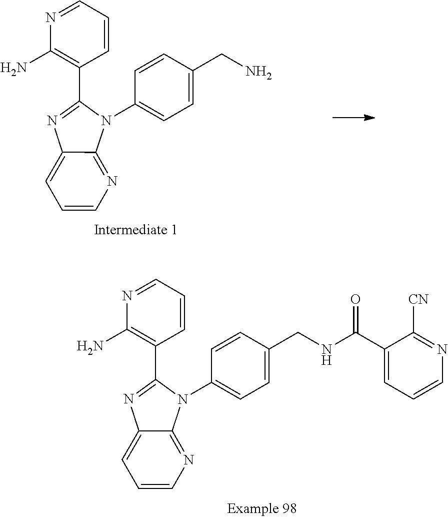 [SMILES: NCc1ccc(-n2c(-c3cccnc3N)nc3cccnc32)cc1 ; N#Cc1ncccc1C(=O)NCc1ccc(-n2c(-c3cccnc3N)nc3cccnc32)cc1]

Following the general procedure of Example 23, the reaction of Intermediate 1 (200 mg, 632 μmol) with 2-cyanonicotinic acid (93.6 mg, 632 μmol) was carried out. After purified by prep-HPLC (column: Waters xbridge 150×25 mm×10 μm; mobile phase: [water (NH₄HCO₃)-ACN]; B %: 20%-50%, 10 min), N-(4-(2-(2-aminopyridin-3-yl)-3H-imidazo[4,5-b]pyridin-3-yl)benzyl)-2-cyanonicotinamide (Example 98, 14.7 mg, yield: 5.2%) was obtained as a yellow solid. MS: m/z=447.2 [M+H]⁺. ¹H NMR (400 MHz, Dimethylsulfoxide-d₆) δ 10.01 (s, 1H), 8.96 (d, J=4.8 Hz, 1H), 8.32-8.27 (m, 2H), 8.18 (d, J=8.0 Hz, 1H), 7.98 (d, J=4.8 Hz, 1H), 7.76 (dd, J=6.4 Hz, 5.2 Hz, 1H), 7.48 (d, J=8.0 Hz, 2H), 7.39-7.35 (m, 3H), 7.20 (d, J=7.6 Hz, 1H), 6.95 (br s, 2H), 6.41 (dd, J=8.0 Hz, 4.8 Hz, 1H), 5.05 (s, 2H).

### Example 99: N-(4-(2-(2-Aminopyridin-3-yl)-3H-imidazo[4,5-b]pyridin-3-yl)benzyl)-3-cyanoisonicotinamide

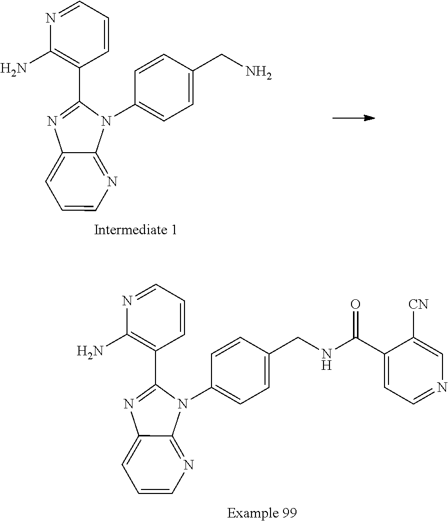 [SMILES: NCc1ccc(-n2c(-c3cccnc3N)nc3cccnc32)cc1 ; N#Cc1cnccc1C(=O)NCc1ccc(-n2c(-c3cccnc3N)nc3cccnc32)cc1]

Following the general procedure of Example 23, the reaction of Intermediate 1 (50 mg, 158 μmol) with 3-cyanopyridine-4-carboxylic acid (23.4 mg, 158 μmol) was carried out. After purified by prep-HPLC (column: Waters Xbridge BEH C18 100×30 mm×10 μm; mobile phase: [water (NH₄HCO₃)-ACN]; B %: 14%-45%, 2 min), N-(4-(2-(2-aminopyridin-3-yl)-3H-imidazo[4,5-b]pyridin-3-yl)benzyl)-3-cyanoisonicotinamide (Example 99, 4.5 mg, yield: 6.4%) was obtained as an off-white lyophilized powder MS: m/z=447.0 [M+H]⁺. ¹H NMR (400 MHz, Chloroform-d) δ 9.12 (s, 1H), 9.07-8.92 (m, 2H), 8.46-8.39 (m, 1H), 8.19-8.10 (m, 1H), 7.94-7.83 (m, 1H), 7.80 (d, J=4.8 Hz, 1H), 7.77-7.56 (m, 2H), 7.39-7.31 (m, 4H), 7.23 (br s, 2H), 6.54-6.43 (m, 1H), 5.15 (s, 2H).

### Example 100: N-(4-(2-(2-Aminopyridin-3-yl)-3H-imidazo[4,5-b]pyridin-3-yl)benzyl)-4-cyanonicotinamide

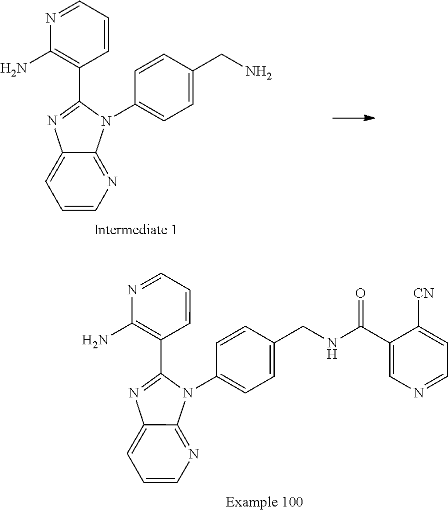 [SMILES: NCc1ccc(-n2c(-c3cccnc3N)nc3cccnc32)cc1 ; N#Cc1ccncc1C(=O)NCc1ccc(-n2c(-c3cccnc3N)nc3cccnc32)cc1]

Following the general procedure of Example 23, the reaction of Intermediate 1 (50 mg, 158 μmol) with 4-cyanopyridine-3-carboxylic acid (23.4 mg, 158 μmol) was carried out. After purified by prep-HPLC (column. Waters Xbridge BEH C18 100×30 mm×10 μm; mobile phase: [water (NH₄HCO₃)-ACN]; B %: 15%-45%, 2 min), N-(4-(2-(2-aminopyridin-3-yl)-3H-imidazo[4,5-b]pyridin-3-yl)benzyl)-4-cyanonicotinamide (Example 100, 8.7 mg, yield: 12.3%) was obtained as an off-white lyophilized powder. MS: m/z=447.1 [M+H]⁺. ¹H NMR (400 MHz, Chloroform-d) δ 9.20 (s, 1H), 9.05 (m, 2H), 8.40 (d, J=4.8 Hz, 1H), 8.15-8.02 (m, 2H), 8.01-7.87 (m, 2H), 7.73-7.59 (m, 3H), 7.41-7.30 (m, 4H), 6.48-6.43 (m, 1H), 5.13 (br s, 2H).

### Example 101: N-(4-(2-(2-Aminopyridin-3-yl)-3H-imidazo[4,5-b]pyridin-3-yl)benzyl)-3-cyanopicolinamide

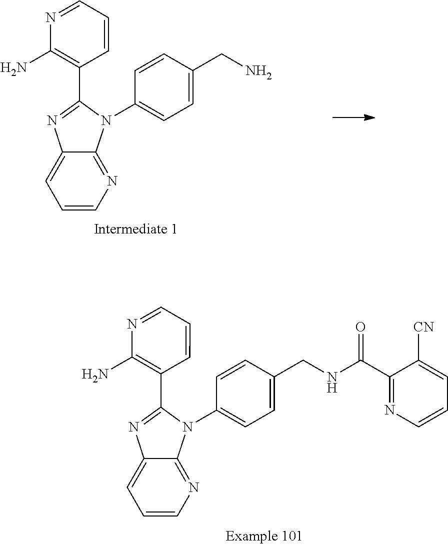 [SMILES: NCc1ccc(-n2c(-c3cccnc3N)nc3cccnc32)cc1 ; N#Cc1cccnc1C(=O)NCc1ccc(-n2c(-c3cccnc3N)nc3cccnc32)cc1]

Following the general procedure of Example 23, the reaction of Intermediate 1 (200 mg, 632 μmol) with 3-cyanopyridine-2-carboxylic acid (93.6 mg, 632 μmol) was carried out. After purified by prep-HPLC (column: Waters xbridge 150×25 mm×10 μm; mobile phase: [water (NH₄HCO₃)-ACN]; B %: 15%-45%, 10 min), N-(4-(2-(2-aminopyridin-3-yl)-3H-imidazo[4,5-b]pyridin-3-yl)benzyl)-3-cyanopicolinamide (Example 101, 6.8 mg, yield: 2.4%) was obtained as a yellow soild. MS: m/z=447.1 [M+H]⁺. ¹H NMR (400 MHz, Methanol-d₄) δ 8.90 (dd, J=4.8, 1.2 Hz, 1H), 8.48 (dd, J=8.0, 1.2 Hz, 1H), 8.29 (dd, J=4.8, 1.6 Hz, 1H), 8.18 (dd, J=8.0, 1.6 Hz, 1H), 7.96 (d, J=5.2 Hz, 1H), 7.77 (dd, J=8.0, 5.2 Hz, 1H), 7.61 (d, J=8.0 Hz, 2H), 7.43-7.37 (m, 3H), 7.31 (dd, J=8.0, 2.0 Hz, 1H), 6.46 (dd, J=7.6, 5.2 Hz, 1H), 5.16 (s, 2H).

### Example 102: 5-(4-(4-(2-(2-Aminopyridin-3-yl)-3H-imidazo[4,5-b]pyridin-3-yl)benzyl)piperazine-1-carbonyl)picolinonitrile

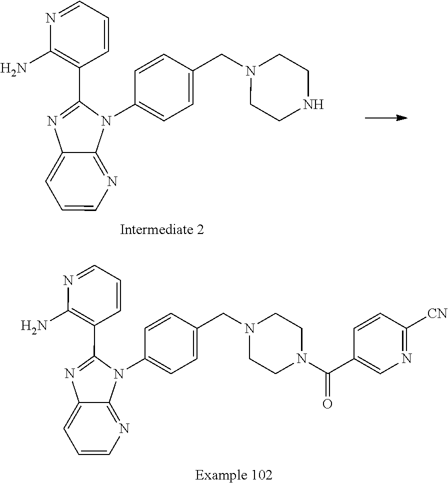 [SMILES: Nc1ncccc1-c1nc2cccnc2n1-c1ccc(CN2CCNCC2)cc1 ; N#Cc1ccc(C(=O)N2CCN(Cc3ccc(-n4c(-c5cccnc5N)nc5cccnc54)cc3)CC2)cn1]

Following the general procedure of Example 23, the reaction of Intermediate 2 (150 mg, 389 μmol) with 6-cyanopyridine-3-carboxylic acid (63.4 mg, 428 μmol) was carried out. After purified by prep-HPLC (column: Waters xbridge 150×25 mm×10 μm; mobile phase: [water (NH₄HCO₃)-ACN]; B %: 19%-49%, 9 min), 5-(4-(4-(2-(2-aminopyridin-3-yl)-3H-imidazo[4,5-b]pyridin-3-yl)benzyl)piperazine-1-carbonyl)picolinonitrile (Example 102, 87 mg, yield: 43%) was obtained as a yellow solid. MS: m/z=516.2 [M+H]⁺. ¹H NMR (400 MHz, Chloroform-d) δ 8.76 (d, J=1.6 Hz, 1H), 8.40 (dd, J=4.8, 1.2 Hz, 1H), 8.11 (dd, J=8.0, 1.2 Hz, 1H), 8.05 (dd, J=4.8, 1.6 Hz, 1H), 7.91 (dd, J=8.0, 2.4 Hz, 1H), 7.77 (d, J=8.4 Hz, 1H), 7.50 (d, J=8.4 Hz, 2H), 7.39-7.27 (m, 3H), 7.07 (dd, J=8.0, 2.0 Hz, 1H), 6.67 (br s, 2H), 6.34 (dd, J=8.0, 5.2 Hz, 1H), 3.90-3.79 (m, 2H), 3.64 (s, 2H), 3.51-3.36 (m, 2H), 2.67-2.43 (m, 4H).

### Example 103: N-(4-(2-(2-Aminopyridin-3-yl)-5-phenyl-3H-imidazo[4,5-b]pyridin-3-yl)benzyl)-2-cyanonicotinamide

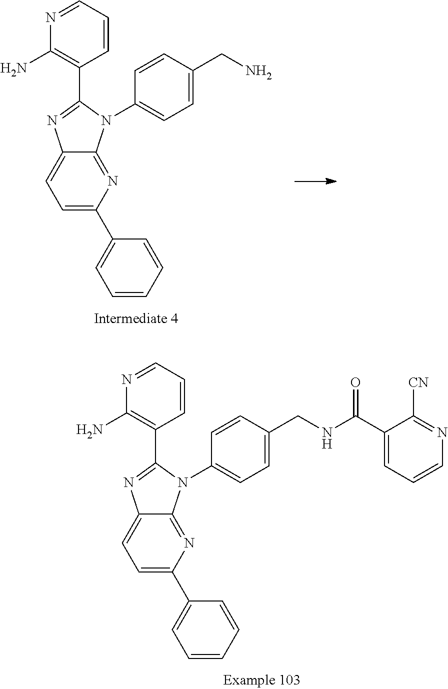 [SMILES: NCc1ccc(-n2c(-c3cccnc3N)nc3ccc(-c4ccccc4)nc32)cc1 ; N#Cc1ncccc1C(=O)NCc1ccc(-n2c(-c3cccnc3N)nc3ccc(-c4ccccc4)nc32)cc1]

Following the general procedure of Example 23, the reaction of Intermediate 4 (120 mg, 306 μmol) with 2-cyanopyridine-3-carboxylic acid (45.3 mg, 306 μmol) was carried out. After purified by prep-HPLC (column: Phenomenex C18 150×25 mm×10 μm; mobile phase: [water (NH₄HCO₃)-ACN]; B %: 35%-65%, 8 min), N-(4-(2-(2-aminopyridin-3-yl)-5-phenyl-3H-imidazo[4,5-b]pyridin-3-yl)benzyl)-2-cyanonicotinamide (Example 103, 38.5 mg, yield: 24%) was obtained as an off-white solid. MS: m/z=523.2 [M+H]⁺. ¹H NMR (400 MHz, Dimethylsulfoxide-d₆) δ 10.01 (s, 1H), 8.96 (dd, J=4.8, 1.2 Hz, 1H), 8.31 (dd, J=8.0, 1.6 Hz, 1H), 8.25 (d, J=8.4 Hz, 1H), 8.02-7.95 (m, 4H), 7.77 (dd, J=8.0, 5.2 Hz, 1H), 7.51 (d, J=8.8 Hz, 2H), 7.48-7.43 (m, 4H), 7.41-7.36 (m, 1H), 7.18 (dd, J=7.6, 1.6 Hz, 1H), 6.91 (br s, 2H), 6.40 (dd, J=7.6, 4.8 Hz, 1H), 5.07 (s, 2H).

### Example 104: N-(4-(2-(2-Aminopyridin-3-yl)-5-phenyl-3H-imidazo[4,5-b]pyridin-3-yl)benzyl)-3-cyanoisonicotinamide

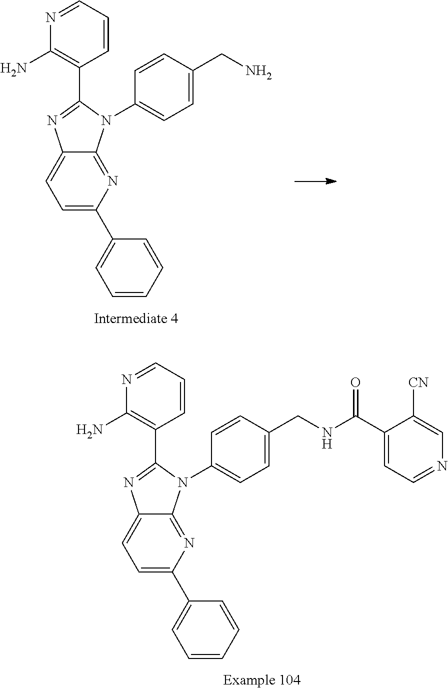 [SMILES: NCc1ccc(-n2c(-c3cccnc3N)nc3ccc(-c4ccccc4)nc32)cc1 ; N#Cc1cnccc1C(=O)NCc1ccc(-n2c(-c3cccnc3N)nc3ccc(-c4ccccc4)nc32)cc1]

Following the general procedure of Example 23, the reaction of Intermediate 4 (50 mg, 127 μmol) with 3-cyanopyridine-4-carboxylic acid (18.9 mg, 127 μmol) was carried out. After purified by prep-HPLC (column. Waters Xbridge BEH C18 100×30 mm×10 μm; mobile phase: [water (NH₄HCO₃)-ACN]; B %: 33%-63%, 2 min), N-(4-(2-(2-aminopyridin-3-yl)-5-phenyl-3H-imidazo[4,5-b]pyridin-3-yl)benzyl)-3-cyanoisonicotinamide (Example 104, 10.5 mg, yield: 16%) was obtained as an off-white solid. MS: m/z=523.2 [M+H]⁺. ¹H NMR (400 MHz, Chloroform-d) δ 9.22-9.03 (m, 3H), 8.96 (d, J=5.6 Hz, 1H), 8.12 (d, J=8.4 Hz, 1H), 7.93-7.88 (m, 2H), 7.80 (d, J=8.4 Hz, 1H), 7.76 (d, J=8.8 Hz, 1H), 7.74-7.70 (m, 1H), 7.61 (d, J=8.0 Hz, 2H), 7.40-7.30 (m, 7H), 6.47 (dd, J=7.2, 6.4 Hz, 1H), 5.19 (s, 2H).

### Example 105: N-(4-(2-(2-Aminopyridin-3-yl)-5-phenyl-3H-imidazo[4,5-b]pyridin-3-yl)benzyl)-4-cyanonicotinamide

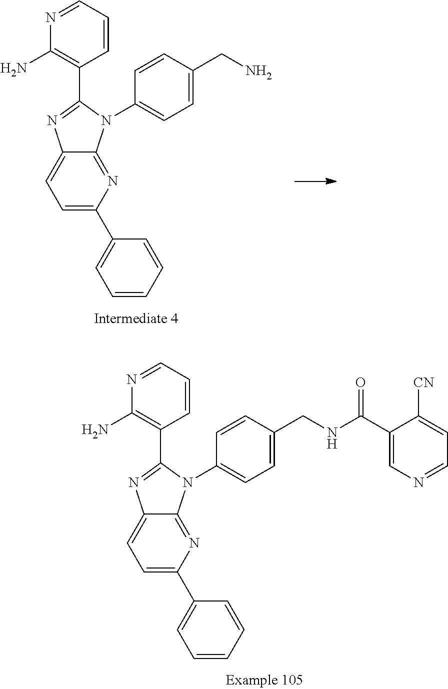 [SMILES: NCc1ccc(-n2c(-c3cccnc3N)nc3ccc(-c4ccccc4)nc32)cc1 ; N#Cc1ccncc1C(=O)NCc1ccc(-n2c(-c3cccnc3N)nc3ccc(-c4ccccc4)nc32)cc1]

Following the general procedure of Example 23, the reaction of Intermediate 4 (50 mg, 127 μmol) with 4-cyanopyridine-3-carboxylic acid (18.9 mg, 127 μmol) was carried out. After purified by prep-HPLC (column: Waters Xbridge BEH C18 100×30 mm×10 μm; mobile phase: [water (NH₄HCO₃)-ACN]; B %: 33%-63%, 2 min), N-(4-(2-(2-aminopyridin-3-yl)-5-phenyl-3H-imidazo[4,5-b]pyridin-3-yl)benzyl)-4-cyanonicotinamide (Example 105, 10.2 mg, yield: 15%) was obtained as an off-white solid. MS: m/z=523.2 [M+H]⁺. ¹H NMR (400 MHz, Chloroform-d) δ 9.23 (s, 1H), 9.06 (d, J=6.0 Hz, 1H), 8.20 (d, J=8.4 Hz, 1H), 7.97 (d, J=8.0 Hz, 2H), 7.87 (d, J=8.0 Hz, 1H), 7.80-7.64 (m, 4H), 7.52-7.37 (m, 7H), 7.25-7.22 (m, 2H), 6.59-6.52 (m, 1H), 5.19 (s, 2H).

### Example 106: N-(4-(2-(2-Aminopyridin-3-yl)-6-phenyl-3H-imidazo[4,5-b]pyridin-3-yl)benzyl)-6-cyanonicotinamide

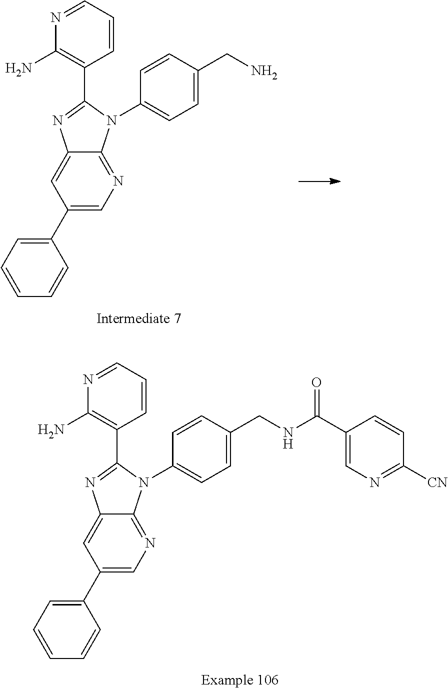 [SMILES: NCc1ccc(-n2c(-c3cccnc3N)nc3cc(-c4ccccc4)cnc32)cc1 ; N#Cc1ccc(C(=O)NCc2ccc(-n3c(-c4cccnc4N)nc4cc(-c5ccccc5)cnc43)cc2)cn1]

Following the general procedure of Example 23, the reaction of Intermediate 7 (150 mg, 382 μmol) with 6-cyanopyridine-3-carboxylic acid (68 mg, 459 μmol) was carried out. After purified by prep-HPLC (column: Waters xbridge 150*25 mm 10 μm; mobile phase: [water (NH₄HCO₃)-ACN]; B %: 30%-60%, 8 min), N-(4-(2-(2-aminopyridin-3-yl)-6-phenyl-3H-imidazo[4,5-b]pyridin-3-yl)benzyl)-6-cyanonicotinamide (Example 106, 52 mg, yield: 26%) was obtained as an off-white solid. MS: m/z=523.2 [M+H]⁺. ¹H NMR (400 MHz, Dimethylsulfoxide-d₆) δ 9.52 (t, J=6.0 Hz, 1H), 9.20 (d, J=1.6 Hz, 1H), 8.61 (d, J=2.0 Hz, 1H), 8.51-8.44 (m, 2H), 8.20 (d, J=8.0 Hz, 1H), 8.01 (dd, J=4.8, 1.6 Hz, 1H), 7.78 (d, J=7.6 Hz, 2H), 7.55-7.49 (m, 4H), 7.46-7.39 (m, 3H), 7.27 (dd, J=7.6, 1.6 Hz, 1H), 6.99 (br s, 2H), 6.43 (dd, J=7.6, 4.8 Hz, 1H), 4.62 (d, J=6.0 Hz, 2H).

### Example 107: N-(1-(4-(2-(2-Aminopyridin-3-yl)-6-phenyl-3H-imidazo[4,5-b]pyridin-3-yl)benzyl)piperidin-4-yl)-2-cyanoisonicotinamide

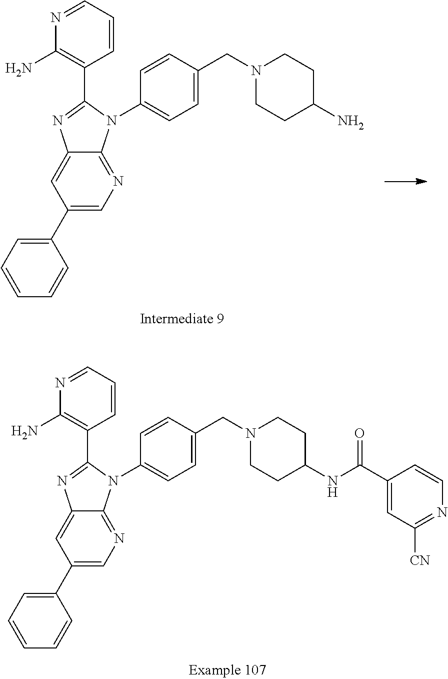 [SMILES: Nc1ncccc1-c1nc2cc(-c3ccccc3)cnc2n1-c1ccc(CN2CCC(N)CC2)cc1 ; N#Cc1cc(C(=O)NC2CCN(Cc3ccc(-n4c(-c5cccnc5N)nc5cc(-c6ccccc6)cnc54)cc3)CC2)ccn1]

Following the general procedure of Example 23, the reaction of Intermediate 9 (150 mg, 315 μmol) with 2-cyanopyridine-4-carboxylic acid (46.7 mg, 315 μmol) was carried out. After purified by prep-HPLC (column: Waters Xbridge 150×25 mm×5 μm; mobile phase: [water (NH₄HCO₃)-ACN]; B %: 35%-65%, 9 min), N-(1-(4-(2-(2-aminopyridin-3-yl)-6-phenyl-3H-imidazo[4,5-b]pyridin-3-yl)benzyl)piperidin-4-yl)-2-cyanoisonicotinamide (Example 107, 117 mg, yield: 60%) was obtained as a white solid. MS: m/z=606.3 [M+H]⁺. ¹H NMR (400 MHz, Dimethylsulfoxide-d₆) δ 8.90 (d, J=4.8 Hz, 1H), 8.74 (d, J=7.6 Hz, 1H), 8.64 (d, J=2.0 Hz, 1H), 8.46 (d, J=2.0 Hz, 1H), 8.37 (s, 1H), 8.07 (dd, J=5.2, 1.6 Hz, 1H), 8.01 (dd, J=4.8, 1.6 Hz, 1H), 7.78 (d, J=7.2 Hz, 2H), 7.55-7.50 (m, 2H), 7.48 (m, J=8.4 Hz, 2H), 7.44-7.39 (m, 3H), 7.20 (dd, J=7.6, 1.6 Hz, 1H), 7.07 (br s, 2H), 6.39 (dd, J=7.6, 4.8 Hz, 1H), 3.84-3.76 (m, 1H), 3.58 (s, 2H), 2.87 (br d, J=11.6 Hz, 2H), 2.16-2.07 (m, 2H), 1.88-1.81 (m, 2H), 1.67-1.58 (m, 2H).

### Example 108: N-(4-(2-(2-Aminopyridin-3-yl)-3H-imidazo[4,5-b]pyridin-3-yl)benzyl)-6-cyanopicolinamide

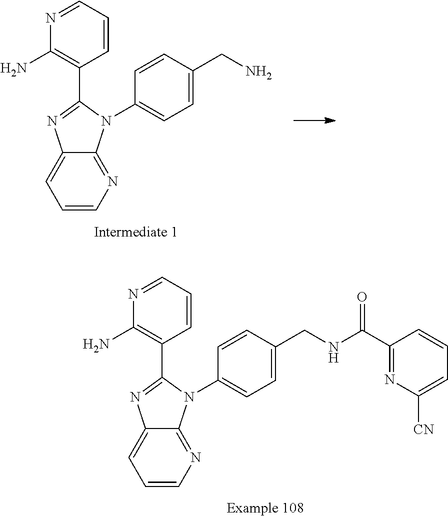 [SMILES: NCc1ccc(-n2c(-c3cccnc3N)nc3cccnc32)cc1 ; N#Cc1cccc(C(=O)NCc2ccc(-n3c(-c4cccnc4N)nc4cccnc43)cc2)n1]

Following the general procedure of Example 23, the reaction of Intermediate 1 (180 mg, 569 μmol) with 6-cyanopicolinic acid (92.7 mg, 626 μmol) was carried out. After purified by prep-HPLC (column: Waters xbridge 150×25 mm 10 μm; mobile phase: [water (NH₄HCO₃)-ACN]; B %: 21%-51%, 8 min), N-(4-(2-(2-aminopyridin-3-yl)-3H-imidazo[4,5-b]pyridin-3-yl)benzyl)-6-cyanopicolinamide (Example 108 130 mg, yield: 50%) was obtained as a light-yellow solid. MS: m/z=447.1 [M+H]⁺. ¹H NMR (400 MHz, Dimethylsulfoxide-d₆) δ 9.60 (t, J=6.4 Hz, 1H), 8.35-8.32 (m, 1H), 8.31-8.28 (m, 1H), 8.28-8.22 (m, 2H), 8.19 (dd, J=8.0, 1.6 Hz, 1H), 7.99 (dd, J=4.8, 2.0 Hz, 1H), 7.47 (d, J=8.4 Hz, 2H), 7.40-7.36 (m, 3H), 7.23 (dd, J=7.6, 2.0 Hz, 1H), 6.96 (br s, 2H), 6.41 (dd, J=7.6, 4.8 Hz, 1H), 4.58 (d, J=6.4 Hz, 2H).

### Example 109: 4-(4-(4-(2-(2-Aminopyridin-3-yl)-3H-imidazo[4,5-b]pyridin-3-yl)benzyl)piperazine-1-carbonyl)picolinonitrile

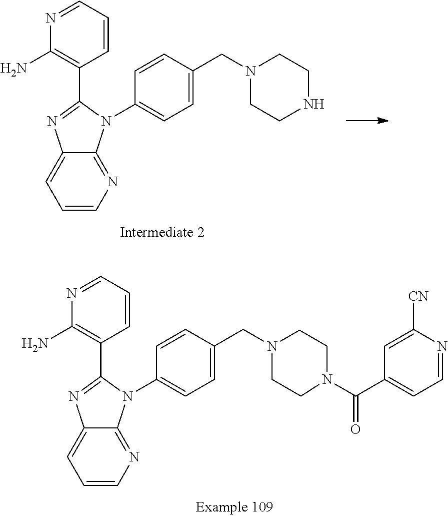 [SMILES: Nc1ncccc1-c1nc2cccnc2n1-c1ccc(CN2CCNCC2)cc1 ; N#Cc1cc(C(=O)N2CCN(Cc3ccc(-n4c(-c5cccnc5N)nc5cccnc54)cc3)CC2)ccn1]

Following the general procedure of Example 23, the reaction of Intermediate 2 (200 mg, 519 μmol) with 2-cyanoisonicotinic acid (76.9 mg, 519 μmol) was carried out. After purified by prep-HPLC (column: Waters xbridge 150×25 mm 10 μm; mobile phase: [water (NH₄HCO₃)-ACN]; B %: 21%-51%, 8 min), (column: Waters xbridge 150×25 mm 10 μm; mobile phase: [water (NH₄HCO₃)-ACN]; B %: 21%-51%, 8 min), 4-(4-(4-(2-(2-aminopyridin-3-yl)-3H-imidazo[4,5-b]pyridin-3-yl)benzyl)piperazine-1-carbonyl)picolinonitrile (Example 109, 110 mg, yield: 41%) was obtained as a light-yellow solid. MS: m/z=516.3 [M+H]⁺. ¹H NMR (400 MHz, Dimethylsulfoxide-d₆) δ 8.84 (d, J=5.2 Hz, 1H), 8.32 (dd, J=4.8, 1.6 Hz, 1H), 8.20 (dd, J=8.0, 1.2 Hz, 1H), 8.11 (s, 1H), 7.98 (dd, J=4.8, 1.6 Hz, 1H), 7.75 (dd, J=4.8, 1.6 Hz, 1H), 7.46 (d, J=8.0 Hz, 2H), 7.41-7.37 (m, 3H), 7.16 (dd, J=7.6, 1.6 Hz, 1H), 7.00 (br s, 2H), 6.38 (dd, J=7.6, 4.8 Hz, 1H), 3.69-3.64 (m, 2H), 3.60 (s, 2H), 3.31-3.27 (m, 2H), 2.60-2.54 (m, 2H), 2.42-2.37 (m, 2H).

### Example 110: 2-(4-(4-(2-(2-Aminopyridin-3-yl)-3H-imidazo[4,5-b]pyridin-3-yl)benzyl)piperazine-1-carbonyl)nicotinonitrile

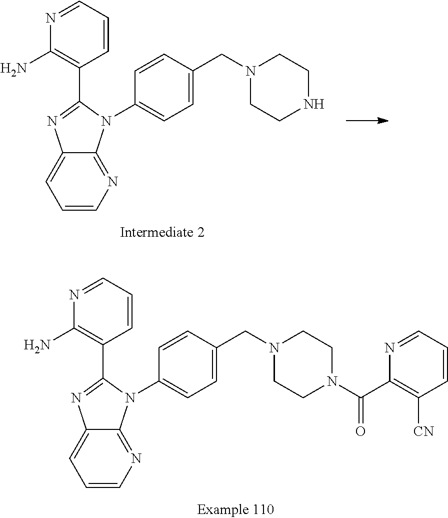 [SMILES: Nc1ncccc1-c1nc2cccnc2n1-c1ccc(CN2CCNCC2)cc1 ; N#Cc1cccnc1C(=O)N1CCN(Cc2ccc(-n3c(-c4cccnc4N)nc4cccnc43)cc2)CC1]

Following the general procedure of Example 23, the reaction of Intermediate 2 (200 mg, 518 μmol) with 3-cyanopicolinic acid (115 mg, 778 μmol) was carried out. After purified by prep-HPLC (column: Waters Xbridge BEH C18 150*25 mm*5 μm; mobile phase: [water (NH₄HCO₃)-ACN]; B %: 15%-45%, 10 min), 2-(4-(4-(2-(2-aminopyridin-3-yl)-3H-imidazo[4,5-b]pyridin-3-yl)benzyl)piperazine-1-carbonyl)nicotinonitrile (Example 110, 10.5 mg, yield: 3.9%) was obtained as a light-yellow powder. MS: m/z=516.0 [M+H]⁺. ¹H NMR (400 MHz, Dimethylsulfoxide-d₆) δ 8.86 (dd, J=4.8, 1.6 Hz, 1H), 8.47 (dd, J=8.0, 1.6 Hz, 1H), 8.32 (dd, J=4.8, 1.2 Hz, 1H), 8.20 (dd, J=8.0, 1.6 Hz, 1H), 7.98 (dd, J=4.8, 2.0 Hz, 1H), 7.70 (dd, J=8.0, 4.8 Hz, 1H), 7.47 (d, J=8.4 Hz, 2H), 7.42-7.34 (m, 3H), 7.15 (dd, J=7.6, 1.6 Hz, 1H), 7.00 (br s, 2H), 6.38 (dd, J=7.6, 4.8 Hz, 1H), 3.75-3.70 (m, 2H), 3.62 (s, 2H), 3.29-3.26 (m, 2H), 2.54-2.52 (m, 2H), 2.43-2.39 (m, 2H).

### Example 111: 5-(4-(4-(2-(2-Aminopyridin-3-yl)-6-phenyl-3H-imidazo[4,5-b]pyridin-3-yl)benzyl)piperazine-1-carbonyl)picolinonitrile

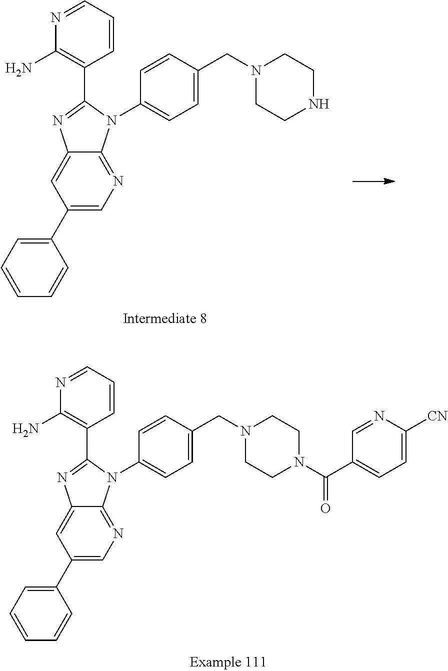 [SMILES: Nc1ncccc1-c1nc2cc(-c3ccccc3)cnc2n1-c1ccc(CN2CCNCC2)cc1 ; N#Cc1ccc(C(=O)N2CCN(Cc3ccc(-n4c(-c5cccnc5N)nc5cc(-c6ccccc6)cnc54)cc3)CC2)cn1]

Following the general procedure of Example 23, the reaction of Intermediate 8 (150 mg, 325 μmol) with 6-cyanopyridine-3-carboxylic acid (58 mg, 389 μmol) was carried out. After purified by prep-HPLC (column: Waters xbridge 150*25 mm 10 μm; mobile phase: [water (NH₄HCO₃)-ACN]; B %: 35%-65%, 9 min), 5-(4-(4-(2-(2-aminopyridin-3-yl)-6-phenyl-3H-imidazo[4,5-b]pyridin-3-yl)benzyl)piperazine-1-carbonyl)picolinonitrile (Example 111, 25.5 mg, yield: 13.3%) was obtained as a yellow solid. MS: m/z=592.1[M+H]⁺. ¹H NMR (400 MHz, Chloroform-d₆) δ 8.78 (s, 1H), 8.64 (d, J=1.6 Hz, 1H), 8.29 (d, J=2.0 Hz, 1H), 8.08 (d, J=4.8 Hz, 1H), 7.93 (dd, J=8.0, 2.0 Hz, 1H), 7.79 (d, J=8.0 Hz, 1H), 7.65 (d, J=7.2 Hz, 1H), 7.55-7.49 (m, 4H), 7.45-7.36 (m, 3H), 7.12 (d, J=7.6 Hz, 1H), 6.78 (br s, 2H), 6.35 (dd, J=7.6, 4.8 Hz, 1H), 3.91-3.79 (m, 2H), 3.65 (s, 2H), 3.52-3.38 (m, 2H), 2.69-2.57 (m, 2H), 2.56-2.45 (m, 2H).

### Example 112: 5-((4-(2-(2-Aminopyridin-3-yl)-5-phenyl-3H-imidazo[4,5-b]pyridin-3-yl)benzyl)amino)pyridazine-3-carbonitrile

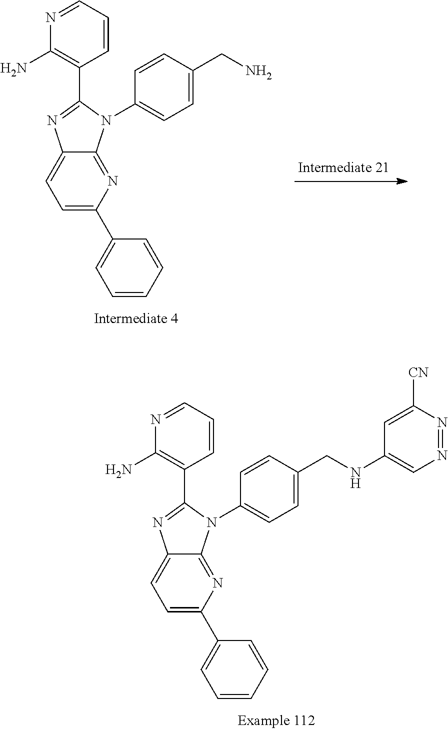 [SMILES: NCc1ccc(-n2c(-c3cccnc3N)nc3ccc(-c4ccccc4)nc32)cc1 ; N#Cc1cc(NCc2ccc(-n3c(-c4cccnc4N)nc4ccc(-c5ccccc5)nc43)cc2)cnn1]

Following the general procedure of Example 47, the reaction of Intermediate 4 (150 mg, 382 μmol) with Intermediate 21 (53 mg, 382 μmol) was carried out. After purified by prep-HPLC (column: Waters xbridge 150*25 mm 10 μm; mobile phase: [water (NH₄HCO₃)-ACN]; B %: 40%-70%, 10 min), 5-((4-(2-(2-aminopyridin-3-yl)-5-phenyl-3H-imidazo[4,5-b]pyridin-3-yl)benzyl)amino)pyridazine-3-carbonitrile (Example 112, 81.9 mg, yield: 43%) was obtained as a yellow solid. MS: m/z=496.2 [M+H]⁺. ¹H NMR (400 MHz, Dimethylsulfoxide-d₆) δ 8.83 (d, J=2.8 Hz, 1H), 8.29-8.19 (m, 2H), 8.04-7.96 (m, 4H), 7.55-7.45 (m, 6H), 7.43-7.37 (m, 1H), 7.32 (d, J=2.8 Hz, 1H), 7.18 (dd, J=7.6, 1.6 Hz, 1H), 6.97 (br s, 2H), 6.38 (dd, J=7.6, 4.8 Hz, 1H), 4.56 (d, J=6.0 Hz, 2H).

### Example 113: N-(4-(2-(2-Aminopyridin-3-yl)-3H-imidazo[4,5-b]pyridin-3-yl)benzyl)-6-cyanopyrimidine-4-carboxamide

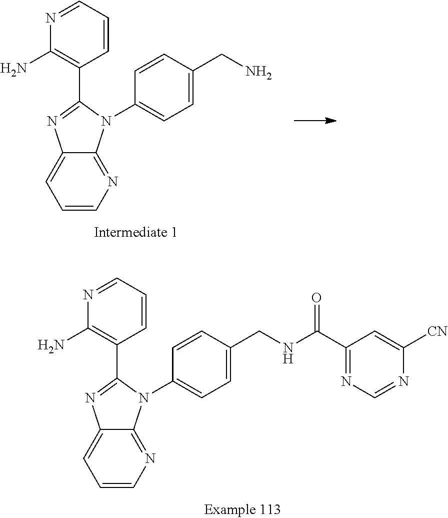 [SMILES: NCc1ccc(-n2c(-c3cccnc3N)nc3cccnc32)cc1 ; N#Cc1cc(C(=O)NCc2ccc(-n3c(-c4cccnc4N)nc4cccnc43)cc2)ncn1]

To a solution of Intermediate 1 (200 mg, 632 μmol), 6-cyanopyrimidine-4-carboxylic acid (94.3 mg, 632 μmol), EDCI (182 mg, 948 μmol) and HOBt (128 mg, 948 μmol) in CH₂Cl₂ (2 mL) was added DIEA (409 mg, 3.16 mmol). The mixture was stirred at 25° C. for 16 hr. The reaction was concentrated under reduced pressure to give a residue. The crude product was purified by prep-HPLC (column: Waters xbridge 150×25 mm 10 μm; mobile phase: [water (NH₄HCO₃)-ACN]; B %: 18%-48%, 8 min), N-(4-(2-(2-aminopyridin-3-yl)-3H-imidazo[4,5-b]pyridin-3-yl)benzyl)-6-cyanopyrimidine-4-carboxamide (Example 113, 57.5 mg, yield: 20%) was obtained as a yellow solid. MS: m/z=448.2 [M+H]⁺. ¹H NMR (400 MHz, Dimethylsulfoxide-d₆) δ 9.89 (t, J=6.4 Hz, 1H), 9.56 (d, J=1.2 Hz, 1H), 8.58 (d, J=1.2 Hz, 1H), 8.29 (dd, J=4.8, 1.6 Hz, 1H), 8.19 (dd, J=8.0, 1.2 Hz, 1H), 7.98 (dd, J=4.8, 1.6 Hz, 1H), 7.47 (d, J=8.4 Hz, 2H), 7.40-7.36 (m, 3H), 7.23 (dd, J=7.6, 2.0 Hz, 1H), 6.94 (br s, 2H), 6.40 (dd, J=8.0, 4.8 Hz, 1H), 4.59 (d, J=6.4 Hz, 2H).

### Example 114: 6-(4-(4-(2-(2-Aminopyridin-3-yl)-3H-imidazo[4,5-b]pyridin-3-yl)benzyl)piperazine-1-carbonyl)pyrimidine-4-carbonitrile

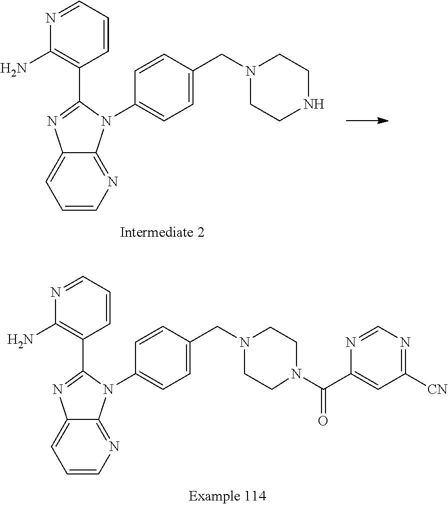 [SMILES: Nc1ncccc1-c1nc2cccnc2n1-c1ccc(CN2CCNCC2)cc1 ; N#Cc1cc(C(=O)N2CCN(Cc3ccc(-n4c(-c5cccnc5N)nc5cccnc54)cc3)CC2)ncn1]

Following the general procedure of Example 113, the reaction of Intermediate 2 (200 mg, 519 μmol) with 6-cyanopyrimidine-4-carboxylic acid (77.4 mg, 519 μmol) was carried out. After purified by prep-HPLC (column: Waters xbridge 150×25 mm 10 μm; mobile phase: [water (NH₄HCO₃)-ACN]; B %: 20%-50%, 8 min), 6-(4-(4-(2-(2-aminopyridin-3-yl)-3H-imidazo[4,5-b]pyridin-3-yl)benzyl)piperazine-1-carbonyl)pyrimidine-4-carbonitrile (Example 114, 37.3 mg, yield: 13%) was obtained as a yellow solid. MS: m/z=517.1 [M+H]⁺. ¹H NMR (400 MHz, Dimethylsulfoxide-d₆) δ 9.45 (s, 1H), 8.36 (s, 1H), 8.33 (dd, J=4.8, 1.6 Hz, 1H), 8.20 (dd, J=8.0, 1.6 Hz, 1H), 7.98 (dd, J=4.8, 1.6 Hz, 1H), 7.47 (d, J=8.0 Hz, 2H), 7.41-7.37 (m, 3H), 7.16 (dd, 1=8.0, 2.0 Hz, 1H), 6.99 (br s, 2H), 6.38 (dd, J=7.6, 4.8 Hz, 1H), 3.73-3.65 (m, 2H), 3.62 (s, 2H), 3.44-3.39 (m, 2H), 2.53-2.51 (m, 2H), 2.42-2.38 (m, 2H).

### Example 115: N-(4-(2-(2-Aminopyridin-3-yl)-5-phenyl-3H-imidazo[4,5-b]pyridin-3-yl)benzyl)-6-cyanopyrimidine-4-carboxamide

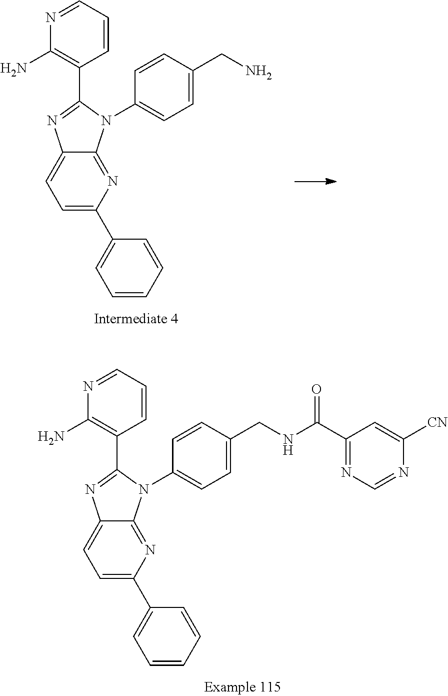 [SMILES: NCc1ccc(-n2c(-c3cccnc3N)nc3ccc(-c4ccccc4)nc32)cc1 ; N#Cc1cc(C(=O)NCc2ccc(-n3c(-c4cccnc4N)nc4ccc(-c5ccccc5)nc43)cc2)ncn1]

Following the general procedure of Example 113, the reaction of Intermediate 4 (200 mg, 510 μmol) with 6-cyanopyrimidine-4-carboxylic acid (76 mg, 510 μmol) was carried out. After purified by prep-HPLC (column: Welch Ultimate C18 150×25 mm×5 μm; mobile phase: [water (FA)-ACN]; B %: 25%-55%, 10 min), N-(4-(2-(2-aminopyridin-3-yl)-5-phenyl-3H-imidazo[4,5-b]pyridin-3-yl)benzyl)-6-cyanopyrimidine-4-carboxamide (Example 115, 24.1 mg, yield: 8%) was obtained as a yellow solid. MS: m/z=523.9 [M+H]⁺. ¹H NMR (400 MHz, Dimethylsulfoxide-d₆) δ9.89 (t, J=6.0 Hz, 1H), 9.57 (d, J=1.2 Hz, 1H), 8.59 (d, J=1.2 Hz, 1H), 8.26 (d, J=8.4 Hz, 1H), 8.03-7.96 (m, 4H), 7.51-7.44 (m, 6H), 7.41-7.37 (m, 1H), 7.21 (dd, J=7.6, 2.0 Hz, 1H), 6.92 (br s, 2H), 6.41 (dd, J=7.6, 4.8 Hz, 1H), 4.63 (d, J=6.0 Hz, 2H).

### Example 116: 6-(4-(4-(2-(2-Aminopyridin-3-yl)-5-phenyl-3H-imidazo[4,5-b]pyridin-3-yl)benzyl)piperazine-1-carbonyl)pyrimidine-4-carbonitrile

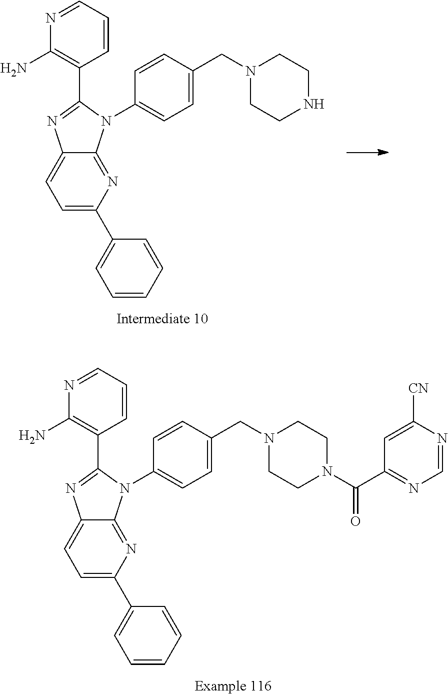 [SMILES: Nc1ncccc1-c1nc2ccc(-c3ccccc3)nc2n1-c1ccc(CN2CCNCC2)cc1 ; N#Cc1cc(C(=O)N2CCN(Cc3ccc(-n4c(-c5cccnc5N)nc5ccc(-c6ccccc6)nc54)cc3)CC2)ncn1]

Following the general procedure of Example 113, the reaction of Intermediate 10 (200 mg, 433 μmol) with 6-cyanopyrimidine-4-carboxylic acid (65 mg, 433 μmol) was carried out. After purified by prep-HPLC (column: Waters xbridge 150×25 mm 10 μm; mobile phase: [water (NH₄HCO₃)-ACN]; B %: 37%-67%, 8 min), 6-(4-(4-(2-(2-aminopyridin-3-yl)-5-phenyl-3H-imidazo[4,5-b]pyridin-3-yl)benzyl)piperazine-1-carbonyl)pyrimidine-4-carbonitrile (Example 116, 46.6 mg, yield. 17%) was obtained as a yellow solid. MS: m/z=593.2 [M+H]⁺. ¹H NMR (400 MHz, Chloroform-d) δ 9.35 (d, J=1.6 Hz, 1H), 8.15 (d, J=8.4 Hz, 1H), 8.03-7.98 (m, 4H), 7.82 (d, J=8.4 Hz, 1H), 7.52 (d, J=8.0 Hz, 1H), 7.45-7.38 (m, 5H), 7.16 (dd, J=7.6, 1.2 Hz, 1H), 7.13-7.02 (m, 2H), 6.38 (dd, J=7.6, 5.2 Hz, 1H), 3.91-3.86 (m, 2H), 3.72-3.62 (m, 4H), 2.68-2.58 (m, 4H).

### Example 117: N-(1-(4-(2-(2-aminopyridin-3-yl)-5-phenyl-3H-imidazo[4,5-b]pyridin-3-yl)benzyl)piperidin-4-yl)-6-cyanopyrimidine-4-carboxamide

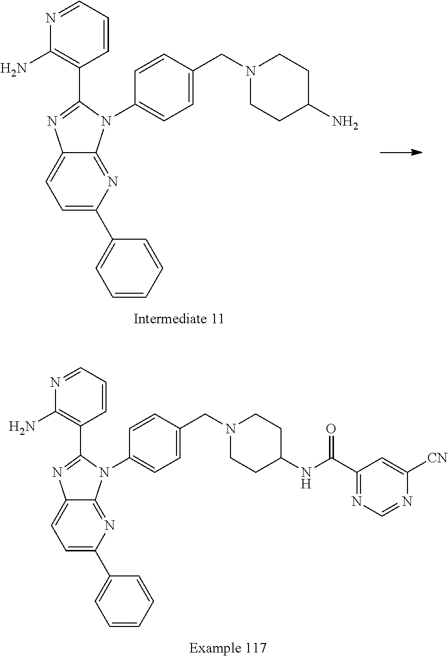 [SMILES: Nc1ncccc1-c1nc2ccc(-c3ccccc3)nc2n1-c1ccc(CN2CCC(N)CC2)cc1 ; N#Cc1cc(C(=O)NC2CCN(Cc3ccc(-n4c(-c5cccnc5N)nc5ccc(-c6ccccc6)nc54)cc3)CC2)ncn1]

Following the general procedure of Example 113, the reaction of Intermediate 11 (200 mg, 421 μmol) with 6-cyanopyrimidine-4-carboxylic acid (63 mg, 421 μmol) was carried out. After purified by prep-HPLC (column: Waters xbridge 150×25 mm 10 μm; mobile phase: [water (NH₄HCO₃)-ACN]; B %: 40%-70%, 8 min), N-(1-(4-(2-(2-aminopyridin-3-yl)-5-phenyl-3H-imidazo[4,5-b]pyridin-3-yl)benzyl)piperidin-4-yl)-6-cyanopyrimidine-4-carboxamide (Example 117, 28.4 mg, yield: 11%) was obtained as a yellow solid. MS: m/z=607.2 [M+H]⁺. ¹H NMR (400 MHz, Dimethylsulfoxide-d₆) δ 9.53 (d, J=1.6 Hz, 1H), 9.06 (d, J=8.0 Hz, 1H), 8.54 (d, J=1.2 Hz, 1H), 8.27 (d, J=8.4 Hz, 1H), 8.05-7.97 (m, 4H), 7.50-7.44 (m, 6H), 7.42-7.37 (m, 1H), 7.16 (dd, J=7.6, 1.6 Hz, 1H), 7.03 (br s, 2H), 6.38 (dd, J=8.0, 5.2 Hz, 1H), 3.88-3.80 (m, 1H), 3.59 (s, 2H), 2.87 (d, J=11.6 Hz, 2H), 2.15-2.08 (m, 2H), 1.80-1.71 (m, 4H).

### Example 118: N-(4-(2-(2-aminopyridin-3-yl)-5-phenyl-3H-imidazo[4,5-b]pyridin-3-yl)benzyl)-3-cyanopicolinamide

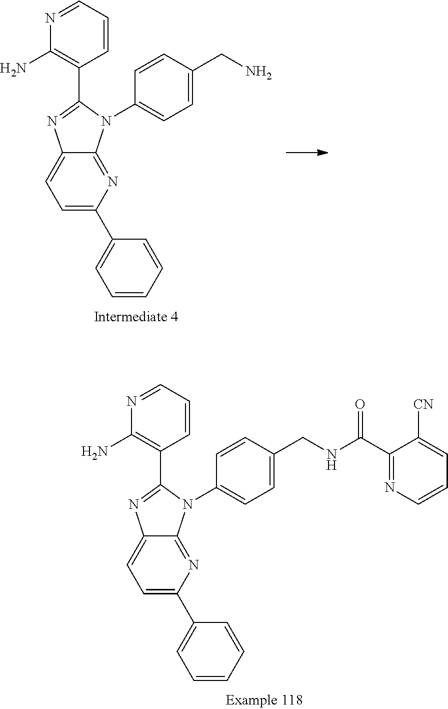 [SMILES: NCc1ccc(-n2c(-c3cccnc3N)nc3ccc(-c4ccccc4)nc32)cc1 ; N#Cc1cccnc1C(=O)NCc1ccc(-n2c(-c3cccnc3N)nc3ccc(-c4ccccc4)nc32)cc1]

Following the general procedure of Example 113, the reaction of Intermediate 4 (200 mg, 510 μmol) with 3-cyanopicolinic acid (76 mg, 510 μmol) was carried out. After purified by prep-HPLC (column: Phenomenex C18 150×25 mm×10 μm; mobile phase: [water (NH₄HCO₃)-ACN]; B %: 36%-66%, 8 min), N-(4-(2-(2-aminopyridin-3-yl)-5-phenyl-3H-imidazo[4,5-b]pyridin-3-yl)benzyl)-3-cyanopicolinamide (Example 118, 19.6 mg, yield: 7%) was obtained as a yellow solid. MS: m/z=523.3 [M+H]⁺. ¹H NMR (400 MHz, Dimethylsulfoxide-d₆) δ 10.52 (s, 1H), 8.92 (dd, J=4.8, 1.6 Hz, 1H), 8.60 (dd, J=8.0, 1.6 Hz, 1H), 8.26 (d, J=8.4 Hz, 1H), 8.02-7.96 (m, 4H), 7.80 (dd, J=7.6, 4.8 Hz, 1H), 7.50-7.44 (m, 6H), 7.41-7.36 (m, 1H), 7.18 (dd, J=7.6, 1.6 Hz, 1H), 6.91 (br s, 2H), 6.40 (dd, J=7.6, 4.8 Hz, 1H), 5.07 (s, 2H).

### Example 119: N-(4-(2-(2-Aminopyridin-3-yl)-3H-imidazo[4,5-b]pyridin-3-yl)benzyl)-2-cyanopyrimidine-5-carboxamide

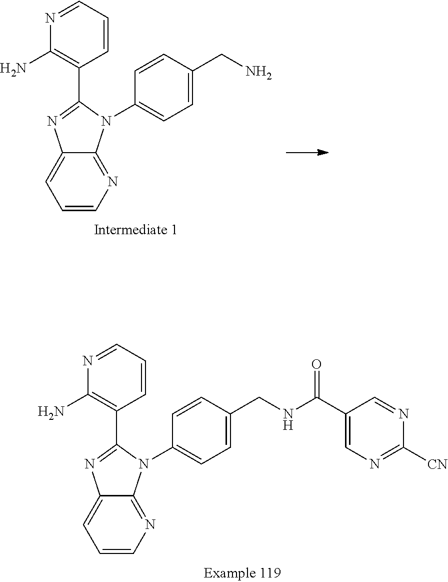 [SMILES: NCc1ccc(-n2c(-c3cccnc3N)nc3cccnc32)cc1 ; N#Cc1ncc(C(=O)NCc2ccc(-n3c(-c4cccnc4N)nc4cccnc43)cc2)cn1]

To a solution of Intermediate 1, 2-cyanopyrimidine-5-carboxylic acid (94.3 mg, 632 μmol) and HATU (361 mg, 948 μmol) in DMF (1 mL) was added DIEA (409 mg, 3.16 mmol). The mixture was stirred at 25° C. for 16 hr. The reaction mixture was filtered to give filter liquor. The filter liquor was purified by prep-HPLC (column: Phenomenex C18 150×25 mm×10 μm; mobile phase: [water (NH₄HCO₃)-ACN]; B %: 18%-48%, 8 min), N-(4-(2-(2-aminopyridin-3-yl)-3H-imidazo[4,5-b]pyridin-3-yl)benzyl)-2-cyanopyrimidine-5-carboxamide (Intermediate 119, 42.5 mg, yield: 15%) was obtained as an off-white lyophilized powder. MS: m/z=448.3 [M+H]⁺. ¹H NMR (400 MHz, Dimethylsulfoxide-d₆) δ 9.66 (t, J=5.6 Hz, 1H), 9.39 (s, 2H), 8.30 (dd, J=4.8, 1.2 Hz, 1H), 8.20 (dd, J=8.0, 1.6 Hz, 1H), 7.99 (dd, J=4.8, 1.6 Hz, 1H), 7.51 (d, J=8.8 Hz, 2H), 7.44-7.36 (m, 3H), 7.24 (dd, J=7.6, 1.6 Hz, 1H), 6.92 (br s, 2H), 6.41 (dd, J=8.0, 5.2 Hz, 1H), 4.63 (d, J=5.6 Hz, 2H).

### Example 120: N-(4-(2-(2-Aminopyridin-3-yl)-5-phenyl-3H-imidazo[4,5-b]pyridin-3-yl)benzyl)-2-cyanopyrimidine-5-carboxamide

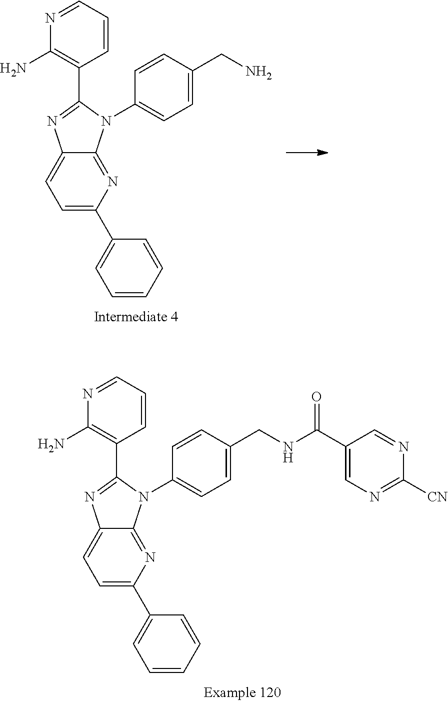 [SMILES: NCc1ccc(-n2c(-c3cccnc3N)nc3ccc(-c4ccccc4)nc32)cc1 ; N#Cc1ncc(C(=O)NCc2ccc(-n3c(-c4cccnc4N)nc4ccc(-c5ccccc5)nc43)cc2)cn1]

To a solution of Intermediate 4 (135 mg, 344 μmol), 2-cyanopyrimidine-5-carboxylic acid (51.3 mg, 344 μmol) and HATU (196 mg, 516 μmol) in DMF (1 mL) was added DIEA (222 mg, 1.72 mmol). The mixture was stirred at 25° C. for 16 hr. The reaction mixture was filtered to give filter liquor. The filter liquor was purified by prep-HPLC (column: Phenomenex C18 150×25 mm×10 μm; mobile phase: [water (NH₄HCO₃)-ACN]; B %: 18%-48%, 8 min), N-(4-(2-(2-aminopyridin-3-yl)-5-phenyl-3H-imidazo[4,5-b]pyridin-3-yl)benzyl)-2-cyanopyrimidine-5-carboxamide (Example 120, 34.7 mg yield: 19.3%) was obtained as a gray solid. MS: m/z=524.3 [M+H]⁺. ¹H NMR (400 MHz, Dimethylsulfoxide-d₆) δ 9.67 (t, J=5.6 Hz, 1H), 9.40 (s, 2H), 8.27 (d, J=8.4 Hz, 1H), 8.05-8.02 (m, 1H), 8.01-7.96 (m, 3H), 7.56-7.52 (m, 2H), 7.51-7.48 (m, 2H), 7.45-7.43 (m, 2H), 7.42-7.37 (m, 1H), 7.23 (dd, J=7.6, 2.0 Hz, 1H), 6.91 (br s, 2H), 6.42 (dd, J=7.6, 4.8 Hz, 1H), 4.67 (d, J=5.6 Hz, 2H).

### Example 121: N-(4-(2-(2-Aminopyridin-3-yl)-3H-imidazo[4,5-b]pyridin-3-yl)benzyl)-6-cyanonicotinamide

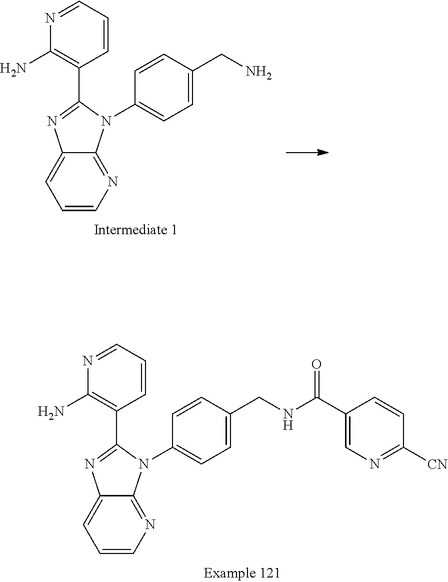 [SMILES: NCc1ccc(-n2c(-c3cccnc3N)nc3cccnc32)cc1 ; N#Cc1ccc(C(=O)NCc2ccc(-n3c(-c4cccnc4N)nc4cccnc43)cc2)cn1]

To a solution of Intermediate 1 and 6-cyanonicotinic acid (84 mg, 570 μmol) in DMF (2 mL) was added HATU (270 mg, 711 μmol) and DIEA (306 mg, 2.4 mmol). The resulting mixture was stirred at 25° C. for 16 hr. The reaction mixture was filtered. The filter liquor was concentrated to dryness to give a residue. The residue was purified by prep-HPLC (column: Waters xbridge 150×25 mm 10 μm; mobile phase: [water (NH₄HCO₃)-ACN]; B %: 16%-46%, 8 min), N-(4-(2-(2-aminopyridin-3-yl)-3H-imidazo[4,5-b]pyridin-3-yl)benzyl)-6-cyanonicotinamide (Example 121, 89.6 mg, yield: 42%) was obtained as a light yellow solid. MS: m/z=447.3 [M+H]^{+ 1}H NMR (400 MHz, Dimethylsulfoxide-d₆) δ 9.54 (t, J=6.0 Hz, 1H), 9.19 (d, J=1.6 Hz, 1H), 8.47 (dd, J=8.0, 2.4 Hz, 1H), 8.30 (dd, J=4.8, 1.6 Hz, 1H), 8.20 (d, J=8.0 Hz, 2H), 7.99 (dd, J=4.8, 2.0 Hz, 1H), 7.50 (d, J=8.4 Hz, 2H), 7.43-7.36 (m, 3H), 7.24 (dd, J=7.6, 1.6 Hz, 1H), 6.94 (br s, 2H), 6.41 (dd, J=7.8, 4.8 Hz, 1H), 4.61 (d, J=6.0 Hz, 2H).

### Example 122, N-(4-(2-(2-Aminopyridin-3-yl)-3H-imidazo[4,5-b]pyridin-3-yl)benzyl)-2-cyanoisonicotinamide

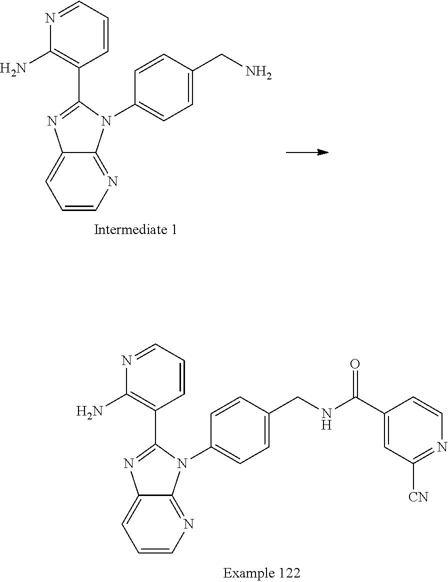 [SMILES: NCc1ccc(-n2c(-c3cccnc3N)nc3cccnc32)cc1 ; N#Cc1cc(C(=O)NCc2ccc(-n3c(-c4cccnc4N)nc4cccnc43)cc2)ccn1]

Following the general procedure of Example 121, the reaction of Intermediate 1 (150 mg, 474 μmol) with 2-cyanoisonicotinic acid (70 mg, 474 μmol) was carried out. After purified by prep-HPLC (column: Waters xbridge 150×25 mm 10 μm; mobile phase: [water (NH₄HCO₃)-ACN]; B %: 26%-56%, 8 min), N-(4-(2-(2-aminopyridin-3-yl)-3H-imidazo[4,5-b]pyridin-3-yl)benzyl)-2-cyanoisonicotinamide (Example 122, 84 mg, yield: 39%) was obtained as a light yellow solid. MS: m/z=447.1 [M+H]⁺. ¹H NMR (400 MHz, Dimethylsulfoxide-d₆) δ 9.57 (t, J=6.0 Hz, 1H), 8.92 (d, J=5.2 Hz, 1H), 8.43 (s, 1H), 8.30 (d, J=4.8 Hz, 1H), 8.19 (d, J=8.0 Hz, 1H), 8.14 (d, J=4.8 Hz, 1H), 8.95 (d, J=4.4 Hz, 1H), 7.49 (d, J=8.0 Hz, 1H), 7.44-7.35 (m, 3H), 7.24 (d, J=7.6 Hz, 1H), 6.94 (br s, 2H), 6.41 (dd, J=7.8, 4.8 Hz, 1H), 4.61 (d, J=6.0 Hz, 2H).

### Example 123: N-(4-(2-(2-Aminopyridin-3-yl)-3H-imidazo[4,5-b]pyridin-3-yl)benzyl)-5-cyanonicotinamide

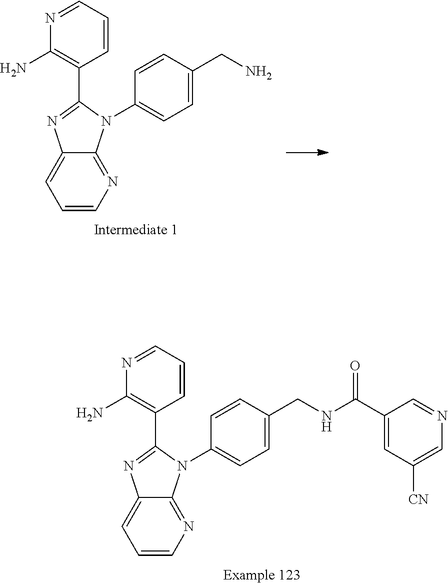 [SMILES: NCc1ccc(-n2c(-c3cccnc3N)nc3cccnc32)cc1 ; N#Cc1cncc(C(=O)NCc2ccc(-n3c(-c4cccnc4N)nc4cccnc43)cc2)c1]

Following the general procedure of Example 121, the reaction of Intermediate 1 (150 mg, 474 μmol) with 5-cyanonicotinic acid (70 mg, 474 μmol) was carried out. After purified by prep-HPLC (column: Waters xbridge 150×25 mm 10 μm; mobile phase: [water (NH₄HCO₃)-ACN]; B %: 14%-44%, 8 min), N-(4-(2-(2-aminopyridin-3-yl)-3H-imidazo[4,5-b]pyridin-3-yl)benzyl)-5-cyanonicotinamide (Example 123, 91.7 mg, yield. 41%) was obtained as a yellow solid. MS: m/z=447.0 [M+H]⁺. ¹H NMR (400 MHz, Dimethylsulfoxide-d₆) δ 9.47 (t, J=6.0 Hz, 1H), 9.29 (d, J=2.0 Hz, 1H), 9.18 (d, J=2.0 Hz, 1H), 8.74 (t, J=2.0 Hz, 1H), 8.31 (dd, J=4.8, 1.2 Hz, 1H), 8.20 (dd, J=8.0, 1.2 Hz, 1H), 7.99 (dd, J=4.8, 2.0 Hz, 1H), 7.49 (d, J=8.4 Hz, 1H), 7.42-7.36 (m, 3H), 7.24 (dd, J=8.0, 2.0 Hz, 1H), 6.94 (br s, 2H), 6.41 (dd, J=8.0, 4.8 Hz, 1H), 4.61 (d, J=6.0 Hz, 2H).

### Example 124: N-(4-(2-(2-Aminopyridin-3-yl)-3H-imidazo[4,5-b]pyridin-3-yl)benzyl)-5-cyanopicolinamide

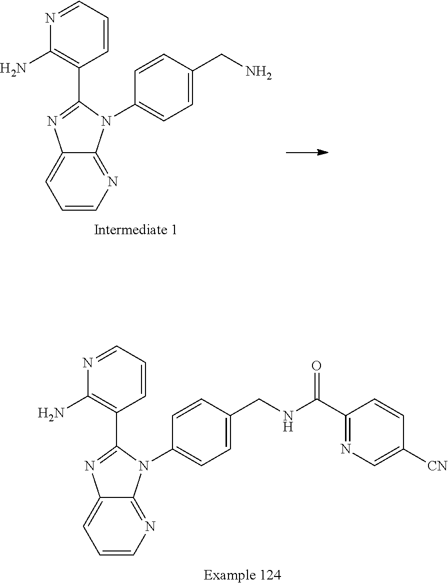 [SMILES: NCc1ccc(-n2c(-c3cccnc3N)nc3cccnc32)cc1 ; N#Cc1ccc(C(=O)NCc2ccc(-n3c(-c4cccnc4N)nc4cccnc43)cc2)nc1]

Following the general procedure of Example 121, the reaction of Intermediate 1 (150 mg, 474 μmol) with 5-cyanopicolinic acid (70 mg, 474 μmol) was carried out. After purified by prep-HPLC (column: Waters xbridge 150×25 mm 10 μm; mobile phase: [water (NH₄HCO₃)-ACN]; B %: 19%-49%, 8 min), N-(4-(2-(2-aminopyridin-3-yl)-3H-imidazo[4,5-b]pyridin-3-yl)benzyl)-5-cyanopicolinamide (Example 124, 96.3 mg, yield: 45%) was obtained as a yellow solid. MS: m/z=447.0 [M+H]⁺. ¹H NMR (400 MHz, Dimethylsulfoxide-d₆) δ 9.69 (t, J=6.4 Hz, 1H), 9.15 (d, J=2.0 Hz, 1H), 8.52 (dd, J=8.0, 2.0 Hz, 1H), 8.29 (dd, J=4.8, 1.2 Hz, 1H), 8.20-8.12 (m, 2H), 7.98 (dd, J=4.8, 1.6 Hz, 1H), 7.46 (d, J=8.4 Hz, 2H), 7.44-7.38 (m, 3H), 7.22 (dd, J=7.6, 1.6 Hz, 1H), 6.96 (br s, 2H), 6.40 (dd, J=7.6, 4.8 Hz, 1H), 4.56 (d, J=6.4 Hz, 2H).

### Example 125: N-(4-(2-(2-Aminopyridin-3-yl)-5-phenyl-3H-imidazo[4,5-b]pyridin-3-yl)benzyl)-5-cyanonicotinamide

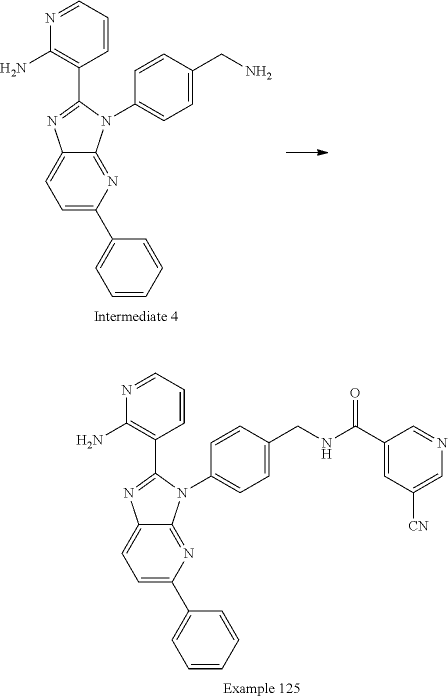 [SMILES: NCc1ccc(-n2c(-c3cccnc3N)nc3ccc(-c4ccccc4)nc32)cc1 ; N#Cc1cncc(C(=O)NCc2ccc(-n3c(-c4cccnc4N)nc4ccc(-c5ccccc5)nc43)cc2)c1]

Following the general procedure of Example 121, the reaction of Intermediate 4 (150 mg, 382 μmol) with 5-cyanonicotinic acid (57 mg, 382 μmol) was carried out. After purified by prep-HPLC (column: Waters xbridge 150×25 mm 10 μm; mobile phase: [water (NH₄HCO₃)-ACN]; B %: 32%-62%, 8 min), N-(4-(2-(2-aminopyridin-3-yl)-5-phenyl-3H-imidazo[4,5-b]pyridin-3-yl)benzyl)-5-cyanonicotinamide (Example 125, 7.7 mg, yield: 3.9%) was obtained as a yellow solid. MS: m/z=523.4 [M+H]⁺. ¹H NMR (400 MHz, Dimethylsulfoxide-d₆) δ 9.46 (t, J=5.6 Hz, 1H), 9.29 (d, J=2.0 Hz, 1H), 9.18 (d, J=2.4 Hz, 1H), 8.74 (t, J=2.0 Hz, 1H), 8.26 (d, J=8.4 Hz, 1H), 8.03-7.96 (m, 4H), 7.54-7.44 (m, 6H), 7.41-7.36 (m, 1H), 7.22 (dd, J=7.6, 1.6 Hz, 1H), 6.92 (br s, 2H), 6.42 (dd, J=7.6, 4.8 Hz, 1H), 4.64 (d, J=5.6 Hz, 2H).

### Example 126: N-(4-(2-(2-Aminopyridin-3-yl)-5-phenyl-3H-imidazo[4,5-b]pyridin-3-yl)benzyl)-5-cyanopicolinamide

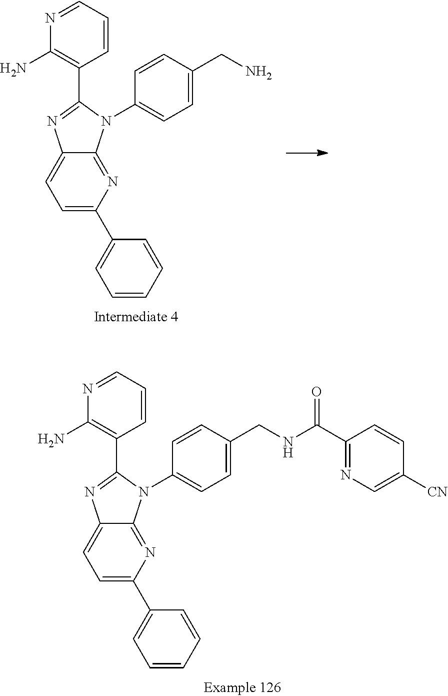 [SMILES: NCc1ccc(-n2c(-c3cccnc3N)nc3ccc(-c4ccccc4)nc32)cc1 ; N#Cc1ccc(C(=O)NCc2ccc(-n3c(-c4cccnc4N)nc4ccc(-c5ccccc5)nc43)cc2)nc1]

Following the general procedure of Example 121, the reaction of Intermediate 4 (150 mg, 382 μmol) with 5-cyanopicolinic acid (57 mg, 382 μmol) was carried out. After purified by prep-HPLC (column: Waters xbridge 150×25 mm 10 μm; mobile phase: [water (NH₄HCO₃)-ACN]; B %: 36%-66%, 8 min), N-(4-(2-(2-aminopyridin-3-yl)-5-phenyl-3H-imidazo[4,5-b]pyridin-3-yl)benzyl)-5-cyanopicolinamide (Example 126, 28.3 mg, yield: 15%) was obtained as a yellow solid. MS: m/z=523.4 [M+H]⁺. ¹H NMR (400 MHz, Dimethylsulfoxide-d₆) δ9.70 (t, J=6.4 Hz, 1H), 9.15 (d, J=1.2 Hz, 1H), 8.54 (dd, J=8.4, 2.0 Hz, 1H), 8.28-8.16 (m, 2H), 8.03-7.96 (m, 4H), 7.49-7.37 (m, 7H), 7.20 (dd, J=7.6, 1.6 Hz, 1H), 6.93 (br s, 2H), 6.41 (dd, J=7.6, 4.8 Hz, 1H), 4.62 (d, J=6.4 Hz, 2H).

### Example 127: 4-((4-(2-(2-Aminopyridin-3-yl)-3H-imidazo[4,5-b]pyridin-3-yl)benzyl)amino)picolinonitrile

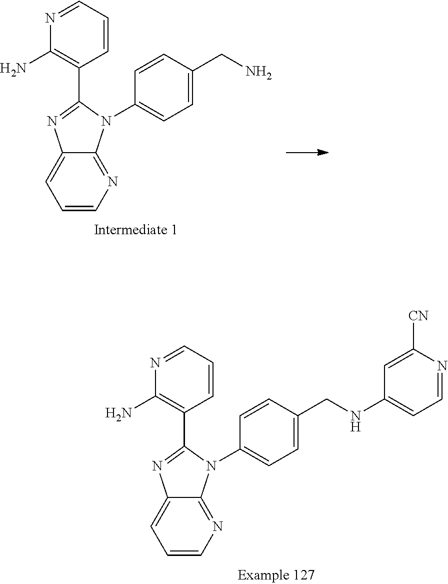 [SMILES: NCc1ccc(-n2c(-c3cccnc3N)nc3cccnc32)cc1 ; N#Cc1cc(NCc2ccc(-n3c(-c4cccnc4N)nc4cccnc43)cc2)ccn1]

To a solution of Intermediate 1 (150 mg, 474 μmol) and 4-fluoropyridine-2-carbonitrile (58 mg, 474 μmol) in DMSO (2 mL) was added DLEA (306 mg, 2.4 mmol). The mixture was stirred at 100° C. for 16 hr. Then the mixture was purified by prep-HPLC (column: Waters xbridge 150×25 mm 10 μm; mobile phase: [water (NH₄HCO₃)-ACN]; B %: 18%-48%, 8 min), 4-((4-(2-(2-aminopyridin-3-yl)-3H-imidazo[4,5-b]pyridin-3-yl)benzyl)amino)picolinonitrile (Example 127, 48.6 mg, yield: 24%) was obtained as a yellow solid. MS: m/z=419.0 [M+H]⁺. ¹H NMR (400 MHz, Dimethylsulfoxide-d₆) δ 8.31 (dd, J=4.8, 1.2 Hz, 1H), 8.20 (dd, J=7.6, 1.2 Hz, 1H), 8.14 (d, J=5.6 Hz, 1H), 7.99 (dd, J=4.8, 2.0 Hz, 1H), 7.76 (t, J=6.0 Hz, 1H), 7.48 (d, J=8.4 Hz, 1H), 7.43-7.36 (m, 3H), 7.17 (dd, J=7.6, 2.0 Hz, 1H), 7.12 (d, J=2.0 Hz, 1H), 6.98 (br s, 2H), 6.79 (d, J=3.6 Hz, 1H), 6.36 (dd, J=7.6, 4.8 Hz, 1H), 4.48 (d, J=6.0 Hz, 2H).

### Example 128: 5-((4-(2-(2-Aminopyridin-3-yl)-3H-imidazo[4,5-b]pyridin-3-yl)benzyl)amino)picolinonitrile

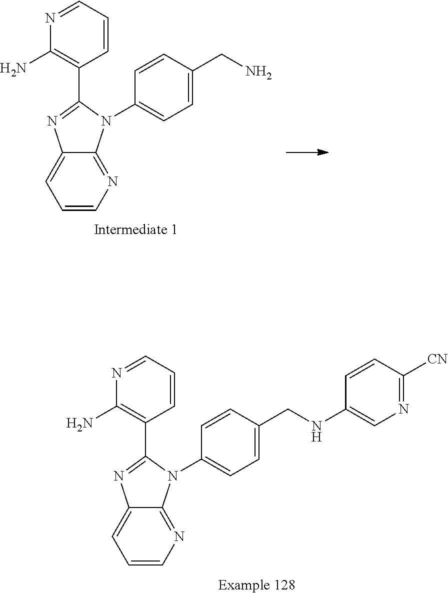 [SMILES: NCc1ccc(-n2c(-c3cccnc3N)nc3cccnc32)cc1 ; N#Cc1ccc(NCc2ccc(-n3c(-c4cccnc4N)nc4cccnc43)cc2)cn1]

Following the general procedure of Example 127, the reaction of Intermediate 1 (150 mg, 474 μmol) with 5-fluoropicolinonitrile (58 mg, 474 μmol) was carried out. After purified by prep-HPLC (column: Waters xbridge 150×25 mm 10 μm; mobile phase: [water (NH₄HCO₃)-ACN]; B %: 18%-48%, 8 min), 5-((4-(2-(2-aminopyridin-3-yl)-3H-imidazo[4,5-b]pyridin-3-yl)benzyl)amino)picolinonitrile (Example 128, 53.1 mg, yield: 26%) was obtained as a yellow solid. MS: m/z=419.1 [M+H]⁺. ¹H NMR (400 MHz, Dimethylsulfoxide-d₆) δ 8.31 (d, J=3.6 Hz, 1H), 8.20 (d, J=8.0 Hz, 1H), 8.12 (d, J=2.8 Hz, 1H), 7.99 (dd, J=4.8, 1.6 Hz, 1H), 7.67-7.60 (m, 2H), 7.49 (d, J=8.0 Hz, 1H), 7.43-7.36 (m, 3H), 7.17 (dd, J=7.6, 1.6 Hz, 1H), 7.01-6.95 (m, 3H), 6.37 (dd, J=8.0, 4.8 Hz, 1H), 4.49 (d, J=6.0 Hz, 2H).

### Example 129: 4-((4-(2-(2-Aminopyridin-3-yl)-5-phenyl-3H-imidazo[4,5-b]pyridin-3-yl)benzyl)amino)picolinonitrile

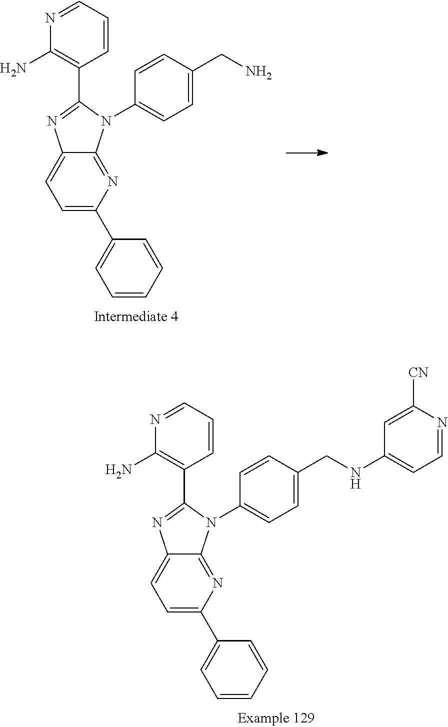 [SMILES: NCc1ccc(-n2c(-c3cccnc3N)nc3ccc(-c4ccccc4)nc32)cc1 ; N#Cc1cc(NCc2ccc(-n3c(-c4cccnc4N)nc4ccc(-c5ccccc5)nc43)cc2)ccn1]

Following the general procedure of Example 127, the reaction of Intermediate 4 (200 mg, 510 μmol) with 4-fluoropyridine-2-carbonitrile (62 mg, 510 μmol) was carried out. After purified by prep-HPLC (column: Waters xbridge 150×25 mm 10 μm; mobile phase: [water (NH₄HCO₃)-ACN]; B %: 35%-65%, 8 min), 4-((4-(2-(2-aminopyridin-3-yl)-5-phenyl-3H-imidazo[4,5-b]pyridin-3-yl)benzyl)amino)picolinonitrile (Example 129, 16.3 mg, yield: 6%) was obtained as a yellow solid. MS: m/z=495.3 [M+H]⁺. ¹H NMR (400 MHz, Dimethylsulfoxide-d₆) δ 8.27 (d, J=8.4 Hz, 1H), 8.14 (d, J=5.6 Hz, 1H), 8.03-7.97 (m, 4H), 7.77 (t, J=6.0 Hz, 1H), 7.53-7.44 (m, 6H), 7.42-7.37 (m, 1H), 7.17 (dd, J=7.6, 1.6 Hz, 1H), 7.12 (d, J=2.0 Hz, 1H), 6.99 (br s, 2H), 6.85-6.79 (m, 1H), 6.37 (dd, J=8.0, 5.2 Hz, 1H), 4.51 (d, J=6.0 Hz, 2H).

### Example 130: 6-((4-(2-(2-Aminopyridin-3-yl)-3H-imidazo[4,5-b]pyridin-3-yl)benzyl)amino)picolinonitrile

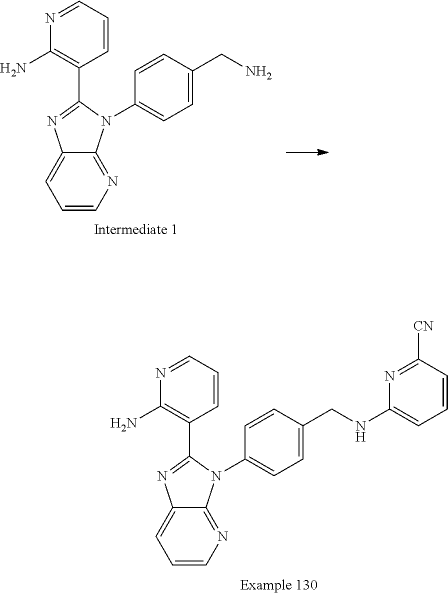 [SMILES: NCc1ccc(-n2c(-c3cccnc3N)nc3cccnc32)cc1 ; N#Cc1cccc(NCc2ccc(-n3c(-c4cccnc4N)nc4cccnc43)cc2)n1]

A mixture of Intermediate 1 (200 mg, 632 μmol), 6-fluoropyridine-2-carbonitrile (92.6 mg, 759 μmol), DLEA (163 mg, 1.26 mmol) in DMSO (1 mL) was stirred under microwave at 120° C. for 1 hr. The reaction mixture was filtered, the filter liquor was purified by prep-HPLC (column: Waters xbridge 150×25 mm 10 μm; mobile phase: [water (NH₄HCO₃)-ACN]; B %: 30%-60%, 9 min), 6-((4-(2-(2-aminopyridin-3-yl)-3H-imidazo[4,5-b]pyridin-3-yl)benzyl)amino)picolinonitrile (Example 130, 74.5 mg, yield: 28%) was obtained as a brown lyophilized powder. MS: m/z=419.1 [M+H]⁺. ¹H NMR (400 MHz, Dimethylsulfoxide-d₆) δ 8.31 (dd, J=4.8, 1.6 Hz, 1H), 8.22-8.16 (m, 1H), 7.98 (dd, J=4.8, 2.0 Hz, 1H), 7.79-7.53 (m, 1H), 7.56 (dd, J=8.4, 7.2 Hz, 1H), 7.46 (d, J=8.4 Hz, 2H), 7.35-7.41 (m, 3H), 7.20 (dd, J=7.6, 2.0 Hz, 1H), 7.11-7.08 (m, 1H), 6.97 (br s, 2H), 6.86 (d, J=8.4 Hz, 1H), 6.38 (dd, J=7.6, 4.8 Hz, 1H), 4.55 (d, J=6.0 Hz, 2H).

### Example 131: 5-((4-(2-(2-Aminopyridin-3-yl)-3H-imidazo[4,5-b]pyridin-3-yl)benzyl)amino)nicotinonitrile

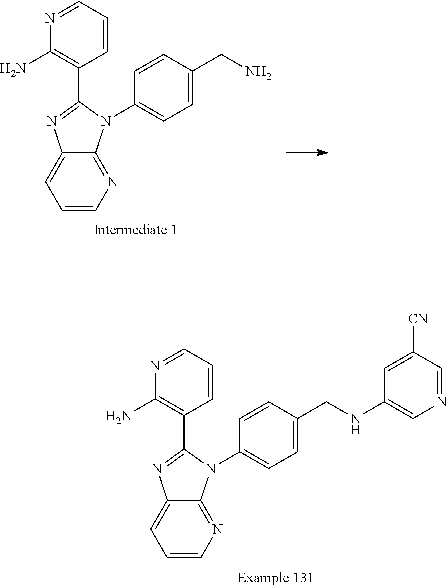 [SMILES: NCc1ccc(-n2c(-c3cccnc3N)nc3cccnc32)cc1 ; N#Cc1cncc(NCc2ccc(-n3c(-c4cccnc4N)nc4cccnc43)cc2)c1]

Following the general procedure of Example 130, the reaction of 3-(3-(4-(aminomethyl)phenyl)-3H-imidazo[4,5-b]pyridin-2-yl)pyridin-2-amine (200 mg, 632 μmol) with 5-fluoropyridine-3-carbonitrile (92.6 mg, 758 μmol) was carried out. After purified by prep-HPLC (column: Waters xbridge 150×25 mm 10 μm; mobile phase: [water (NH₄HCO₃)-ACN]; B %: 25%-55%, 10 min), 5-((4-(2-(2-aminopyridin-3-yl)-3H-imidazo[4,5-b]pyridin-3-yl)benzyl)amino)nicotinonitrile (Example 131, 5.0 mg, yield: 1.9%) was obtained as a yellow solid. MS: m/z=419.1 [M+H]⁺. ¹H NMR (400 MHz, Dimethylsulfoxide-d₆) δ 8.36-8.30 (m, 2H), 8.19 (d, J=8.0 Hz, 1H), 8.13 (s, 1H), 8.00-7.97 (m, 1H), 7.50 (d, J=8.4 Hz, 2H), 7.43-7.36 (m, 3H), 7.30 (s, 1H), 7.20-7.12 (m, 2H), 7.00 (br s, 2H), 6.35 (dd, J=7.6, 4.8 Hz, 1H), 4.44 (d, J=6.0 Hz, 2H).

### Example 132: 2-((4-(2-(2-Aminopyridin-3-yl)-3H-imidazo[4,5-b]pyridin-3-yl)benzyl)amino)isonicotinonitrile

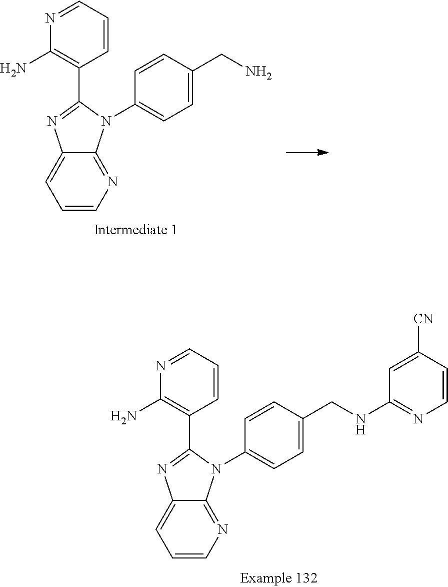 [SMILES: NCc1ccc(-n2c(-c3cccnc3N)nc3cccnc32)cc1 ; N#Cc1ccnc(NCc2ccc(-n3c(-c4cccnc4N)nc4cccnc43)cc2)c1]

To a solution of Intermediate 1 (200 mg, 632 μmol) and 2-chloropyridine-4-carbonitrile (87.6 mg, 632 μmol) in NMP (2 mL) was added DIEA (245 mg, 1.90 mmol). The mixture was stirred under microwave at 160° C. for 1 hr. The reaction mixture was filtered to give filter liquor. The filter liquor was purified by prep-HPLC (column: Welch Xtimate C18 150×25 mm×5 μm; mobile phase: [water (NH₃H₂O)-ACN]; B %: 22%-52%, 8 min), 2-((4-(2-(2-aminopyridin-3-yl)-3H-imidazo[4,5-b]pyridin-3-yl)benzyl)amino)isonicotinonitrile (Example 132, 12.7 mg, yield: 4.8%) was obtained as an off-white lyophilized powder. MS: m/z=419.1 [M+H]⁺. ¹H NMR (400 MHz, Dimethylsulfoxide-d₆) δ 8.30 (d, J=6.0 Hz, 1H), 8.22-8.16 (m, 2H), 8.01-7.95 (m, 1H), 7.74-7.71 (m, 1H), 7.45 (d, J=8.4 Hz, 2H), 7.40-7.35 (m, 3H), 7.20 (d, J=7.6 Hz, 1H), 6.95 (br s, 2H), 6.90 (s, 1H), 6.83 (d, J=5.2 Hz, 1H), 6.38 (dd, J=7.6, 4.8 Hz, 1H), 4.59 (d, J=6.0 Hz, 2H).

### Example 133: 6-((4-(2-(2-Aminopyridin-3-yl)-3H-imidazo[4,5-b]pyridin-3-yl)benzyl)amino)pyrazine-2-carbonitrile

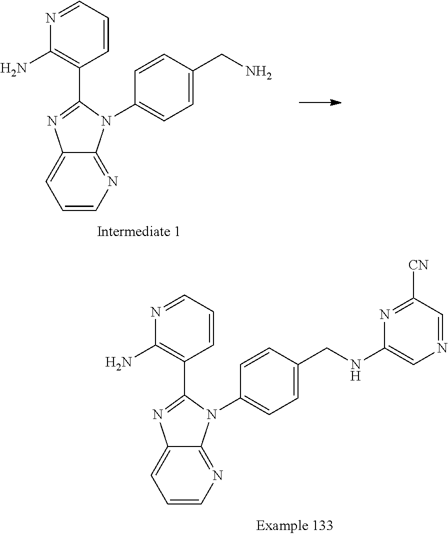 [SMILES: NCc1ccc(-n2c(-c3cccnc3N)nc3cccnc32)cc1 ; N#Cc1cncc(NCc2ccc(-n3c(-c4cccnc4N)nc4cccnc43)cc2)n1]

To a solution of Intermediate 1 (200 mg, 632 μmol) and 6-chloropyrazine-2-carbonitrile (88.2 mg, 632 μmol) in NMP (2 mL) was added DIEA (245 mg, 1.9 mmol). The mixture was stirred under microwave at 130° C. for 0.5 hr. The reaction mixture was filtered to give filter liquor. The filter liquor was purified by prep-HPLC (column: Waters Xbridge BEH C18 100×30 mm×10 μm; mobile phase: [water (ammonia hydroxide v/v)-ACN]; B %. 22%-52%, 10 min), 6-((4-(2-(2-aminopyridin-3-yl)-3H-imidazo[4,5-b]pyridin-3-yl)benzyl)amino)pyrazine-2-carbonitrile (Example 133, 15.6 mg, yield: 5.9%) was obtained as a yellow lyophilized powder. MS: m/z=420.1 [M+H]⁺. ¹H NMR (400 MHz, Dimethylsulfoxide-d₆) δ 8.40-8.27 (m, 3H), 8.24-8.16 (m, 2H), 8.00-7.98 (m, 1H), 7.49 (d, J=8.0 Hz, 2H), 7.43-7.37 (m, 3H), 7.25-7.17 (m, 1H), 6.97 (br s, 2H), 6.35 (dd, J=7.6, 4.8 Hz, 1H), 4.57 (d, J=6.0 Hz, 2H).

### Example 134: 2-((4-(2-(2-Aminopyridin-3-yl)-3H-imidazo[4,5-b]pyridin-3-yl)benzyl)amino)pyrimidine-4-carbonitrile

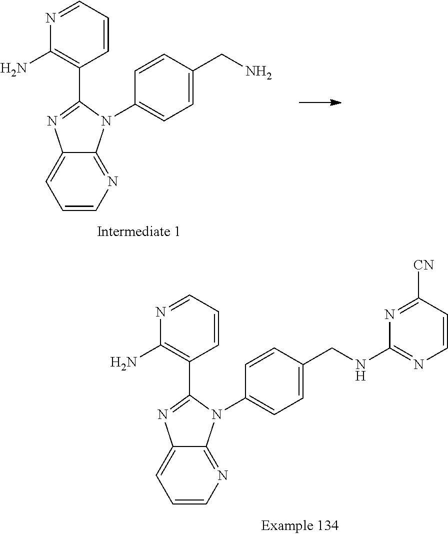 [SMILES: NCc1ccc(-n2c(-c3cccnc3N)nc3cccnc32)cc1 ; N#Cc1ccnc(NCc2ccc(-n3c(-c4cccnc4N)nc4cccnc43)cc2)n1]

Following the general procedure of Example 133, the reaction of Intermediate 1 (200 mg, 632 μmol) with 2-chloropyrimidine-4-carbonitrile (88.2 mg, 632 μmol) was carried out. After purified by prep-HPLC (column: Waters Xbridge BEH C18 100×30 mm×10 μm; mobile phase: [water (ammonia hydroxide v/v)-ACN]; B %: 32%-62%, 10 min), 2-((4-(2-(2-aminopyridin-3-yl)-3H-imidazo[4,5-b]pyridin-3-yl)benzyl)amino)pyrimidine-4-carbonitrile (Example 134, 6.4 mg, yield: 2.4%) was obtained as a yellow lyophilized powder. MS: m/z=420.0 [M+H]⁺. ¹H NMR (400 MHz, Dimethylsulfoxide-d₆) δ 8.59 (d, J=4.8 Hz, 1H), 8.51-8.47 (m, 1H), 8.32-8.29 (m, 1H), 8.20 (dd, J=8.0, 1.6 Hz, 1H), 7.98 (dd, J=4.8, 2.0 Hz, 1H), 7.46-7.42 (m, 2H), 7.40-7.35 (m, 3H), 7.20 (dd, J=7.6, 1.6 Hz, 1H), 7.15 (d, J=4.8 Hz, 1H), 6.98 (br s, 2H), 6.38 (dd, J=7.6, 5.2 Hz, 1H), 4.62-4.53 (m, 2H).

### Example 135: 6-((4-(2-(2-aminopyridin-3-yl)-3H-imidazo[4,5-b]pyridin-3-yl)benzyl)amino)pyrimidine-4-carbonitrile

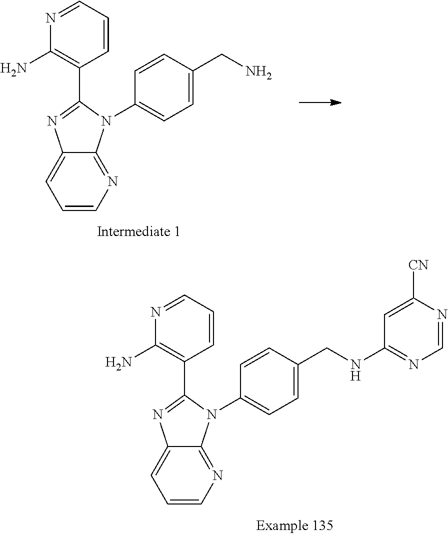 [SMILES: NCc1ccc(-n2c(-c3cccnc3N)nc3cccnc32)cc1 ; N#Cc1cc(NCc2ccc(-n3c(-c4cccnc4N)nc4cccnc43)cc2)ncn1]

Following the general procedure of Example 133, the reaction of Intermediate 1 (150 mg, 474 μmol) with 6-chloropyrimidine-4-carbonitrile (99.2 mg, 711 μmol) was carried out. After purified by prep-HPLC (column: Phenomenex C18 150×25 mm×10 μm; mobile phase: [water (NH₄HCO₃)-ACN]; B %: 23%-53%, 8 min), 6-((4-(2-(2-aminopyridin-3-yl)-3H-imidazo[4,5-b]pyridin-3-yl)benzyl)amino)pyrimidine-4-carbonitrile (Example 135, 27.1 mg, yield: 13.6%) was obtained as a black brown lyophilized powder. MS: m/z=420.0 [M+H]⁺. ¹H NMR (400 MHz, Dimethylsulfoxide-d₆) δ 8.70-8.60 (m, 1H), 8.54 (s, 1H), 8.30 (dd, J=4.8, 1.2 Hz, 1H), 8.19 (dd, J=8.0, 1.2 Hz, 1H), 7.98 (dd, J=4.8, 1.6 Hz, 1H), 7.45 (d, J=8.0 Hz, 2H), 7.42-7.36 (m, 3H), 7.25-7.18 (m, 1H), 7.08 (s, 1H), 6.93 (br s, 2H), 6.39 (dd, J=7.6, 4.8 Hz, 1H), 4.66 (d, J=4.8 Hz, 2H).

### Example 136: 6-((4-(2-(2-Aminopyridin-3-yl)-5-phenyl-3H-imidazo[4,5-b]pyridin-3-yl)benzyl)amino)pyrimidine-4-carbonitrile

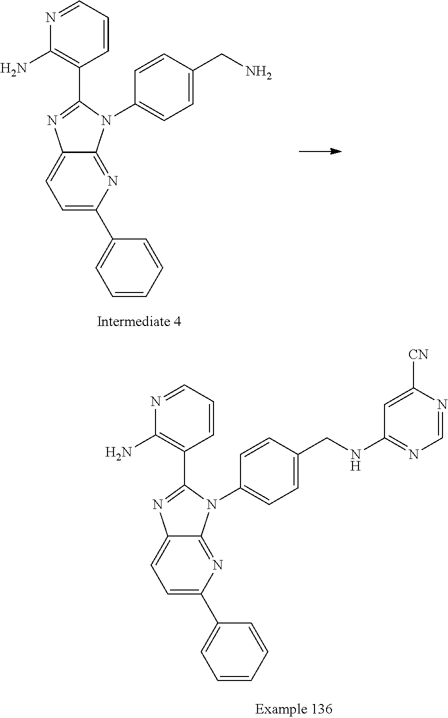 [SMILES: NCc1ccc(-n2c(-c3cccnc3N)nc3ccc(-c4ccccc4)nc32)cc1 ; N#Cc1cc(NCc2ccc(-n3c(-c4cccnc4N)nc4ccc(-c5ccccc5)nc43)cc2)ncn1]

A mixture of Intermediate 4 (150 mg, 382 μmol), 6-chloropyrimidine-4-carbonitrile (80.0 mg, 573 μmol), DIEA (148 mg, 1.15 mmol) in NMP (2 mL) was stirred under microwave at 130° C. for 0.5 hr. The reaction mixture was filtered to give filter liquor. The filter liquor was purified by prep-HPLC (column: Waters xbridge 150×25 mm×10 μm; mobile phase: [water (NH₄HCO₃)-ACN]; B %: 41%-71%, 8 min), 6-((4-(2-(2-aminopyridin-3-yl)-5-phenyl-3H-imidazo[4,5-b]pyridin-3-yl)benzyl)amino)pyrimidine-4-carbonitrile (Example 136, 20 mg, yield: 10.6%) was obtained as a black brown lyophilized powder. MS: m/z=496.1 [M+H]⁺. ¹H NMR (400 MHz, Dimethylsulfoxide-d₆) δ 8.69-8.62 (m, 1H), 8.55 (s, 1H), 8.26 (d, J=8.4 Hz, 1H), 8.03-7.96 (m, 4H), 7.49-7.43 (m, 6H), 7.42-7.37 (m, 1H), 7.23-7.18 (m, 1H), 7.09 (s, 1H), 6.95 (br s, 2H), 6.40 (dd, J=7.6, 4.8 Hz, 1H), 4.70 (d, J=5.6 Hz, 2H).

### Example 137: 4-(4-(4-(2-(2-Aminopyridin-3-yl)-6-phenyl-3H-imidazo[4,5-b]pyridin-3-yl)benzyl)piperazine-1-carbonyl)picolinonitrile

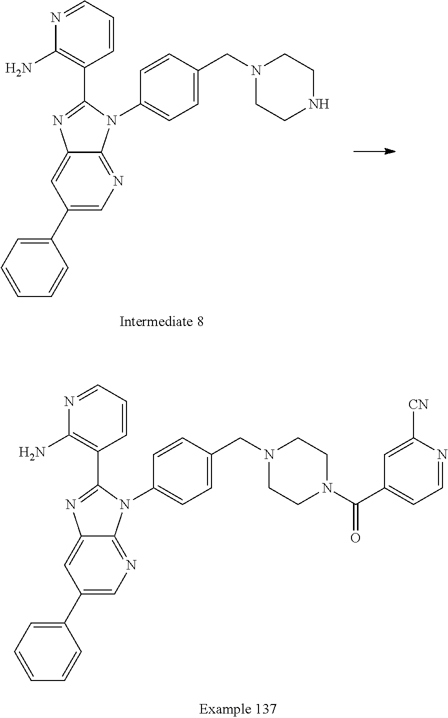 [SMILES: Nc1ncccc1-c1nc2cc(-c3ccccc3)cnc2n1-c1ccc(CN2CCNCC2)cc1 ; N#Cc1cc(C(=O)N2CCN(Cc3ccc(-n4c(-c5cccnc5N)nc5cc(-c6ccccc6)cnc54)cc3)CC2)ccn1]

Following the general procedure of Example 23, the reaction of Intermediate 8 (150 mg, 324 μmol) with 2-cyanoisonicotinic acid (48 mg, 324 μmol) at 25° C. for 1 hr. After purified by triturated with 2 mL MeOH at 25° C. for 1 hr, 4-(4-(4-(2-(2-aminopyridin-3-yl)-6-phenyl-3H-imidazo[4,5-b]pyridin-3-yl)benzyl)piperazine-1-carbonyl)picolinonitrile (Example 137, 34.3 mg, yield: 18%) was obtained as an off-white solid. MS: m/z=592.3 [M+H]⁺. 1H NMR (400 MHz, Dimethylsulfoxide-d₆) δ 8.84 (d, J=4.8 Hz, 1H), 8.63 (d, J=2.0 Hz, 1H), 8.46 (d, J=2.0 Hz, 1H), 8.11 (s, 1H), 8.00 (dd, J=4.8, 1.6 Hz, 1H), 7.81-7.72 (m, 3H), 7.55-7.47 (m, 4H), 7.45-7.40 (m, 3H), 7.19 (dd, J=7.6, 1.6 Hz, 1H), 7.04 (br s, 2H), 6.39 (dd, J=7.6, 4.8 Hz, 1H), 3.72-3.64 (m, 2H), 3.62 (s, 2H), 3.31-3.27 (m, 2H), 2.54-2.51 (m, 2H), 2.43-2.37 (m, 2H).

### Example 138: 6-(4-(4-(2-(2-Aminopyridin-3-yl)-5-phenyl-3H-imidazo[4,5-b]pyridin-3-yl)benzyl)piperazine-1-carbonyl)picolinonitrile

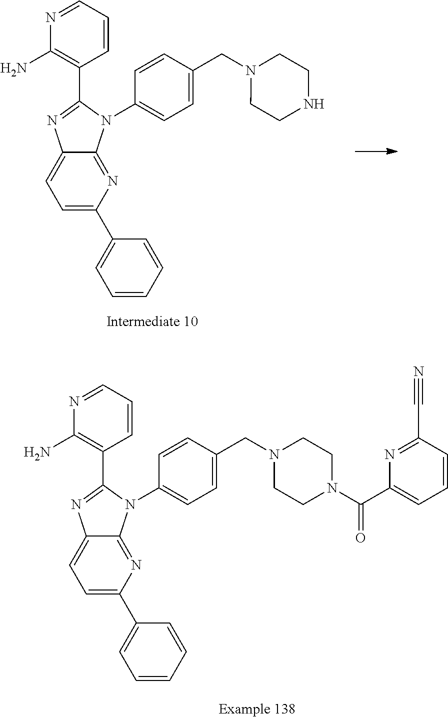 [SMILES: Nc1ncccc1-c1nc2ccc(-c3ccccc3)nc2n1-c1ccc(CN2CCNCC2)cc1 ; N#Cc1cccc(C(=O)N2CCN(Cc3ccc(-n4c(-c5cccnc5N)nc5ccc(-c6ccccc6)nc54)cc3)CC2)n1]

To a solution of Intermediate 10 (200 mg, 433 μmol), 6-cyanopicolinic acid (65.4 mg, 442 μmol) in DMF (2 mL) was added EDCI (115 mg, 602 μmol), DIEA (208 mg, 1.61 mmol) and HOBt (81.4 mg, 602 μmol). The mixture was degassed and purged with N₂ three times and stirred at 25° C. for 16 h under N₂. The reaction mixture was filtered and concentrated under reduced pressure to give a residue. The residue was purified by prep-HPLC (column: Phenomenex C18 150*25 mm*10 μm; mobile phase: [water (NH₄HCO₃)-ACN]; B %: 16%-46%, 8 min), 6-(4-(4-(2-(2-aminopyridin-3-yl)-5-phenyl-3H-imidazo[4,5-b]pyridin-3-yl)benzyl)piperazine-1-carbonyl)picolinonitrile (Example 138, 71.4 mg, yield: 28%) was obtained as a light-yellow solid. MS: m/z=592.1 [M+H]⁺. ¹H NMR (400 MHz, Dimethylsulfoxide-d₆) δ 8.27 (d, J=8.4 Hz, 1H), 8.22-8.17 (m, 1H), 8.13 (d, J=7.6 Hz, 1H), 8.06-7.95 (m, 4H), 7.90 (d, J=7.6 Hz, 1H), 7.54-7.42 (m, 6H), 7.42-7.35 (m, 1H), 7.15 (dd, J=7.6, 2.0 Hz, 1H), 7.02 (br s, 2H), 6.39 (dd, J=7.6, 4.8 Hz, 1H), 3.73-3.68 (m, 2H), 3.64 (s, 2H), 3.44-3.35 (m, 2H), 2.54-2.51 (m, 2H), 2.46-2.39 (m, 2H).

### Example 139: 5-(4-(4-(2-(2-Aminopyridin-3-yl)-5-phenyl-3H-imidazo[4,5-b]pyridin-3-yl)benzyl)piperazine-1-carbonyl)nicotinonitrile

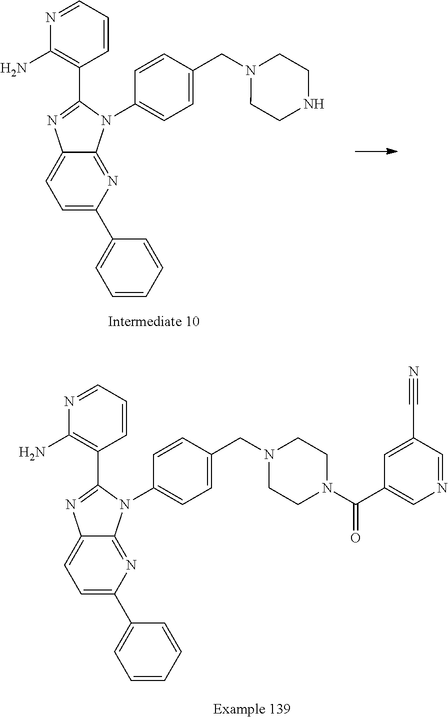 [SMILES: Nc1ncccc1-c1nc2ccc(-c3ccccc3)nc2n1-c1ccc(CN2CCNCC2)cc1 ; N#Cc1cncc(C(=O)N2CCN(Cc3ccc(-n4c(-c5cccnc5N)nc5ccc(-c6ccccc6)nc54)cc3)CC2)c1]

Following the general procedure of Example 138, the reaction of Intermediate 10 (150 mg, 325 μmol) with 5-cyanonicotinic acid (49 mg, 331 μmol) was carried out. After purified by prep-HPLC (column: Waters xbridge 150*25 mm 10 μm; mobile phase: [water (NH₄HCO₃)-ACN]; B %: 34%-64%, 8 min), 5-(4-(4-(2-(2-aminopyridin-3-yl)-5-phenyl-3H-imidazo[4,5-b]pyridin-3-yl)benzyl)piperazine-1-carbonyl)nicotinonitrile (Example 139, 86 mg, yield: 45%) was obtained as a white solid. MS: m/z=592.4 [M+H]⁺. ¹H NMR (400 MHz, Dimethylsulfoxide-d₆) δ 9.10 (d, J=2.0 Hz, 1H), 8.89 (d, J=2.0 Hz, 1H), 8.43-8.40 (m, 1H), 8.27 (d, J=8.0 Hz, 1H), 8.06-7.94 (m, 4H), 7.53-7.43 (m, 6H), 7.42-7.37 (m, 1H), 7.15 (dd, J=7.6, 1.6 Hz, 1H), 7.01 (br s, 2H), 6.39 (dd, J=7.6, 4.8 Hz, 1H), 3.72-3.65 (m, 2H), 3.63 (s, 2H), 3.41-3.35 (m, 2H), 2.55-2.51 (m, 2H), 2.46-2.40 (m, 2H).

### Example 140: 2-(4-(4-(2-(2-Aminopyridin-3-yl)-5-phenyl-3H-imidazo[4,5-b]pyridin-3-yl)benzyl)piperazine-1-carbonyl)isonicotinonitrile

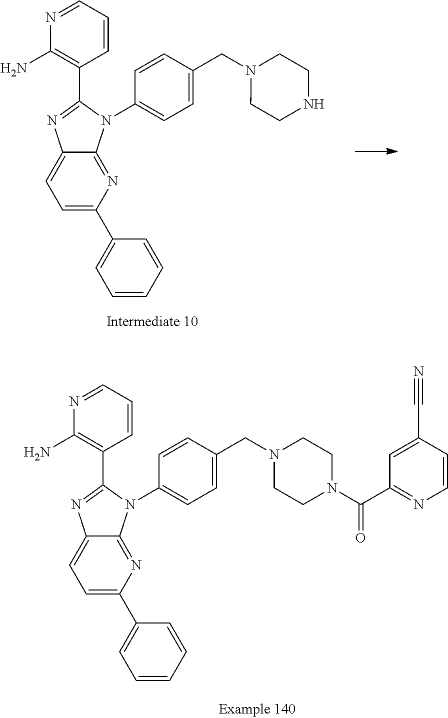 [SMILES: Nc1ncccc1-c1nc2ccc(-c3ccccc3)nc2n1-c1ccc(CN2CCNCC2)cc1 ; N#Cc1ccnc(C(=O)N2CCN(Cc3ccc(-n4c(-c5cccnc5N)nc5ccc(-c6ccccc6)nc54)cc3)CC2)c1]

Following the general procedure of Example 138, the reaction of Intermediate 10 (150 mg, 375 μmol) with 4-cyanopyridine-2-carboxylic acid (48.1 mg, 325 μmol) was carried out. After purified by prep-HPLC (column: Waters xbridge 150×25 mm 10 μm; mobile phase: [water (NH₄HCO₃)-ACN]; B %: 36%-66%, 8 min), 2-(4-(4-(2-(2-aminopyridin-3-yl)-5-phenyl-3H-imidazo[4,5-b]pyridin-3-yl)benzyl)piperazine-1-carbonyl)isonicotinonitrile (Example 140, 39.3 mg, yield: 20%) was obtained as a yellow solid. MS: m/z=592.1 [M+H]⁺. ¹H NMR (400 MHz, Chloroform-d) δ 8.76 (d, J=4.8 Hz, 1H), 8.15 (d, J=8.4 Hz, 1H), 8.02-7.99 (m, 3H), 7.95 (s, 1H), 7.82 (d, J=8.4 Hz, 1H), 7.59-7.52 (m, 3H), 7.48-7.44 (m, 3H), 7.43-7.41 (m, 2H), 7.41-7.33 (m, 2H), 7.18 (dd, J=7.6, 1.2 Hz, 1H), 6.40 (dd, J=8.0, 5.2 Hz, 1H), 3.96-3.87 (m, 2H), 3.73-3.69 (m, 4H), 2.72-2.68 (m, 2H), 2.62-2.58 (m, 2H).

### Example 141: 4-(4-(4-(2-(2-Aminopyridin-3-yl)-5-phenyl-3H-imidazo[4,5-b]pyridin-3-yl)benzyl)piperazine-1-carbonyl)pyrimidine-2-carbonitrile

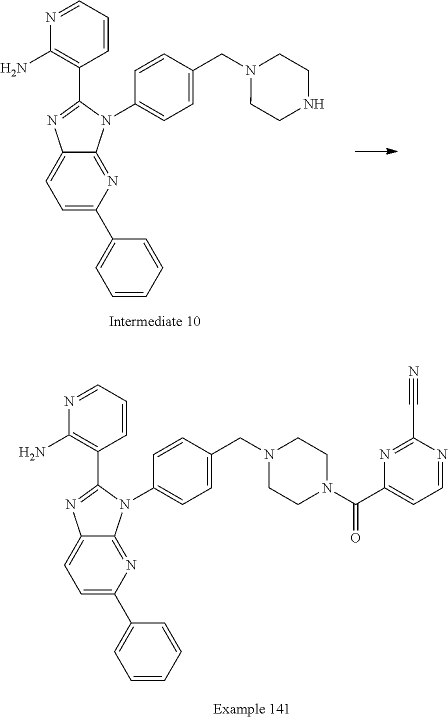 [SMILES: Nc1ncccc1-c1nc2ccc(-c3ccccc3)nc2n1-c1ccc(CN2CCNCC2)cc1 ; N#Cc1nccc(C(=O)N2CCN(Cc3ccc(-n4c(-c5cccnc5N)nc5ccc(-c6ccccc6)nc54)cc3)CC2)n1]

Following the general procedure of Example 138, the reaction of Intermediate 10 (200 mg, 433 μmol) with 2-cyanopyrimidine-4-carboxylic acid (64.6 mg, 433 μmol) was carried out. After purified by prep-HPLC (column: Waters xbridge 150×25 mm 10 μm; mobile phase: [water (NH₄HCO₃)-ACN]; B %: 37%-67%, 8 min), 4-(4-(4-(2-(2-aminopyridin-3-yl)-5-phenyl-3H-imidazo[4,5-b]pyridin-3-yl)benzyl)piperazine-1-carbonyl)pyrimidine-2-carbonitrile (Example 141, 55.9 mg, yield: 21%) was obtained as a yellow solid. MS: m/z=593.1 [M+H]⁺. ¹H NMR (400 MHz, Chloroform-d)) δ 9.00 (d, J=5.2 Hz, 1H), 8.14 (d, J=8.4 Hz, 1H), 8.07 (br s, 1H), 8.01 (d, J=7.2 Hz, 2H), 7.86 (d, J=5.2 Hz, 1H), 7.81 (d, J=8.4 Hz, 1H), 7.54-7.49 (m, 2H), 7.47-7.41 (m, 4H), 7.40-7.34 (m, 1H), 7.11 (d, J=8.0 Hz, 1H), 6.70 (br s, 2H), 6.43-6.34 (m, 1H), 3.90-3.83 (m, 2H), 3.70-3.64 (m, 4H), 2.69-2.57 (m, 4H).

### Example 142: 6-(4-(4-(2-(2-Aminopyridin-3-yl)-5-phenyl-3H-imidazo[4,5-b]pyridin-3-yl)benzyl)piperazine-1-carbonyl)nicotinonitrile

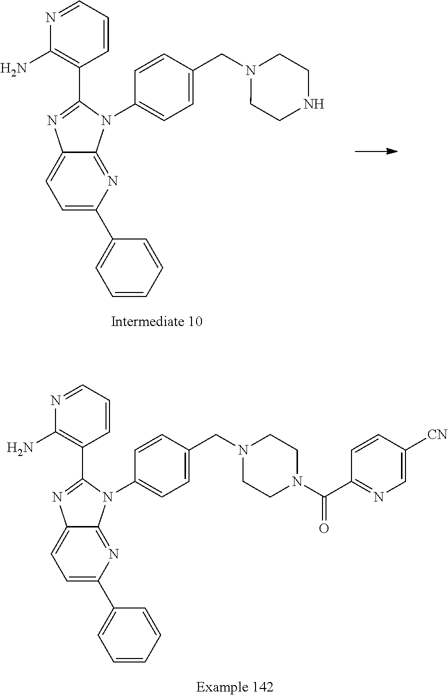 [SMILES: Nc1ncccc1-c1nc2ccc(-c3ccccc3)nc2n1-c1ccc(CN2CCNCC2)cc1 ; N#Cc1ccc(C(=O)N2CCN(Cc3ccc(-n4c(-c5cccnc5N)nc5ccc(-c6ccccc6)nc54)cc3)CC2)nc1]

Following the general procedure of Example 138, the reaction of Intermediate 10 (150 mg, 325 μmol) with 5-cyanopicolinic acid (53 mg, 357 μmol) was carried out. After purified by prep-HPLC (column: Waters xbridge 150*25 mm 10 μm; mobile phase: [water (NH₄HCO₃)-ACN]; B %: 35%-65%, 9 min), 6-(4-(4-(2-(2-aminopyridin-3-yl)-5-phenyl-3H-imidazo[4,5-b]pyridin-3-yl)benzyl)piperazine-1-carbonyl)nicotinonitrile (Example 142, 49 mg, yield: 26%) was obtained as a white solid. MS: m/z=592.2 [M+H]⁺. ¹H NMR (400 MHz, Chloroform-d) δ 8.85 (s, 1H), 8.14 (d, J=8.4 Hz, 1H), 8.08 (dd, J=8.4, 2.0 Hz, 1H), 8.06-7.98 (m, 3H), 7.81 (d, J=8.4 Hz, 2H), 7.52 (d, J=8.4 Hz, 2H), 7.47-7.35 (m, 5H), 7.12 (dd, J=7.6, 1.6 Hz, 1H), 6.82 (br s, 2H), 6.37 (dd, J=7.6, 4.8 Hz, 1H), 3.88 (t, J=4.4 Hz, 1H), 3.67 (s, 2H), 3.63 (t, J=4.8 Hz, 2H), 2.66 (t, J=4.4 Hz, 2H), 2.56 (t, J=4.8 Hz, 2H).

### Example 143: 3-(4-(4-(2-(2-Aminopyridin-3-yl)-5-phenyl-3H-imidazo[4,5-b]pyridin-3-yl)benzyl)piperazine-1-carbonyl)picolinonitrile

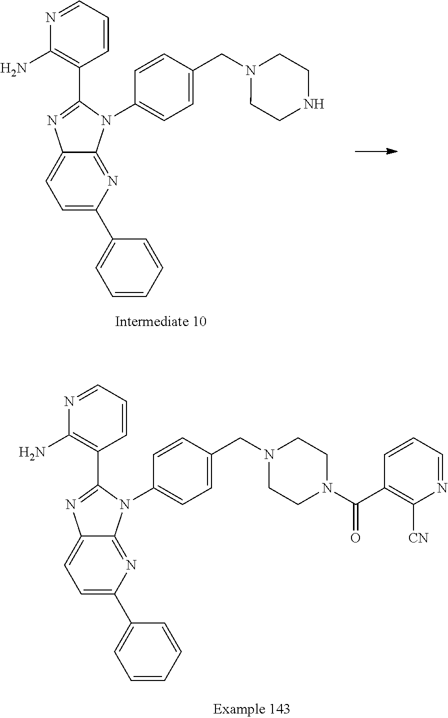 [SMILES: Nc1ncccc1-c1nc2ccc(-c3ccccc3)nc2n1-c1ccc(CN2CCNCC2)cc1 ; N#Cc1ncccc1C(=O)N1CCN(Cc2ccc(-n3c(-c4cccnc4N)nc4ccc(-c5ccccc5)nc43)cc2)CC1]

Following the general procedure of Example 138, the reaction of Intermediate 10 (150 mg, 325 μmol) with 2-cyanonicotinic acid (53 mg, 357 μmol) was carried out. After purified by prep-HPLC (column: Phenomenex C18 150*25 mm*10 μm; mobile phase: [water (NH₄HCO₃)-ACN]; B %: 35%-65%, 8 min), (3-(4-(4-(2-(2-aminopyridin-3-yl)-5-phenyl-3H-imidazo[4,5-b]pyridin-3-yl)benzyl)piperazine-1-carbonyl)picolinonitrile (Example 143, 23.5 mg, yield: 12%) was obtained as a light-yellow solid. MS: m/z=592.2 [M+H]⁺. ¹H NMR (400 MHz, Dimethylsulfoxide-d₆) δ 8.82 (d, J=4.8 Hz, 1H), 8.26 (d, J=8.4 Hz, 1H), 8.09 (dd, J=8.0, 1.2 Hz, 1H), 8.05-7.95 (m, 4H), 7.83 (dd, J=8.0, 4.8 Hz, 1H), 7.53-7.35 (m, 7H), 7.15 (dd, J=7.6, 1.6 Hz, 1H), 7.02 (br s, 2H), 6.39 (dd, J=7.6, 4.8 Hz, 1H), 3.79-3.69 (m, 2H), 3.64 (s, 2H), 3.32-3.28 (m, 2H), 2.56-2.52 (m, 2H), 2.47-2.40 (m, 2H).

### Example 144: 4-(4-(4-(2-(2-Aminopyridin-3-yl)-5-phenyl-3H-imidazo[4,5-b]pyridin-3-yl)benzyl)piperazine-1-carbonyl)nicotinonitrile

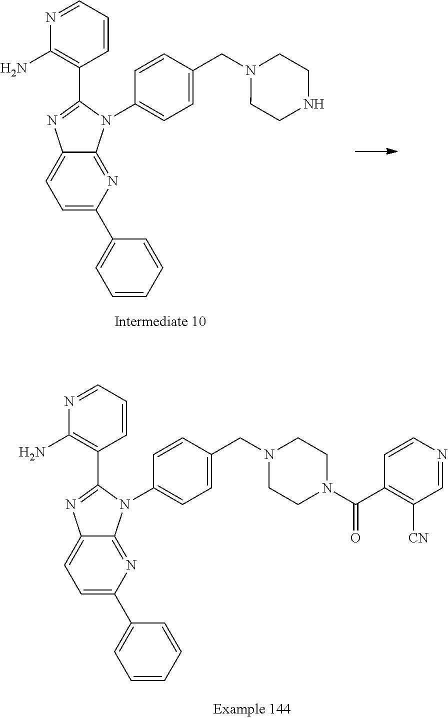 [SMILES: Nc1ncccc1-c1nc2ccc(-c3ccccc3)nc2n1-c1ccc(CN2CCNCC2)cc1 ; N#Cc1cnccc1C(=O)N1CCN(Cc2ccc(-n3c(-c4cccnc4N)nc4ccc(-c5ccccc5)nc43)cc2)CC1]

Following the general procedure of Example 138, the reaction of Intermediate 10 (212 mg, 460 μmol) with 3-cyanopyridine-4-carboxylic acid (75 mg, 506 μmol) was carried out. After purified by prep-HPLC (column: Phenomenex C18 150×25 mm×10 μm; mobile phase: [water (NH₄HCO₃)-ACN]; B %: 33%-63%, 8 min), 4-(4-(4-(2-(2-aminopyridin-3-yl)-5-phenyl-3H-imidazo[4,5-b]pyridin-3-yl)benzyl)piperazine-1-carbonyl)nicotinonitrile (Example 144, 17.0 mg, yield: 6.3%) was obtained as a yellow lyophilized powder. MS: m/z=592.3 [M+H]⁺. ¹H NMR (400 MHz, Dimethylsulfoxide-d₆) δ 9.14 (s, 1H), 8.94 (d, J=4.8 Hz, 1H), 8.27 (d, J=8.4 Hz, 1H), 8.04-8.00 (m, 2H), 8.00-7.96 (m, 2H), 7.65 (d, J=5.2 Hz, 1H), 7.52-7.48 (m, 2H), 7.47-7.43 (m, 4H), 7.42-7.37 (m, 1H), 7.15 (dd, J=7.6, 2.0 Hz, 1H), 7.01 (br s, 2H), 6.39 (dd, J=7.6, 4.8 Hz, 1H), 3.76-3.69 (m, 2H), 3.64 (s, 2H), 3.28-3.25 (m, 2H), 2.54-2.52 (m, 2H), 2.45-2.41 (m, 2H).

### Example 145: 3-(4-(4-(2-(2-Aminopyridin-3-yl)-5-phenyl-3H-imidazo[4,5-b]pyridin-3-yl)benzyl)piperazine-1-carbonyl)isonicotinonitrile

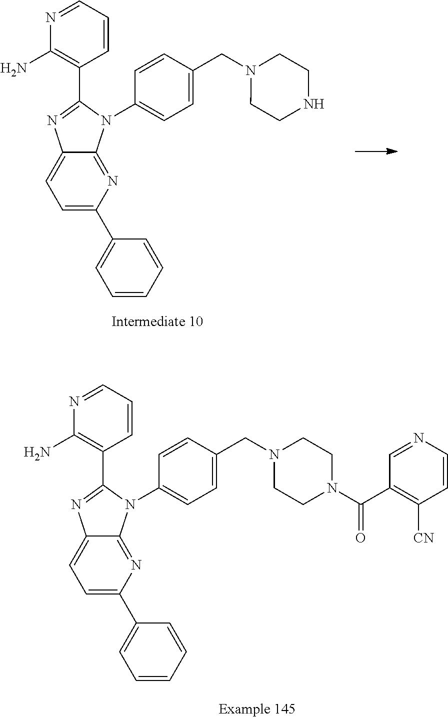 [SMILES: Nc1ncccc1-c1nc2ccc(-c3ccccc3)nc2n1-c1ccc(CN2CCNCC2)cc1 ; N#Cc1ccncc1C(=O)N1CCN(Cc2ccc(-n3c(-c4cccnc4N)nc4ccc(-c5ccccc5)nc43)cc2)CC1]

Following the general procedure of Example 138, the reaction of Intermediate 10 (150 mg, 325 μmol) with 4-cyanonicotinic acid (53 mg, 357 μmol) was carried out. After purified by prep-HPLC (column: Phenomenex C18 150*25 mm*10 μm; mobile phase: [water (NH₄HCO₃)-ACN]; B %: 35%-65%, 8 min), 3-(4-(4-(2-(2-aminopyridin-3-yl)-5-phenyl-3H-imidazo[4,5-b]pyridin-3-yl)benzyl)piperazine-1-carbonyl)isonicotinonitrile (Example 145, 7.2 mg, yield. 3.7%) was obtained as a light-yellow solid. MS: m/z=592.2 [M+H]⁺. ¹H NMR (400 MHz, Dimethylsulfoxide-d₆) δ 8.90 (d, J=5.2 Hz, 1H), 8.86 (s, 1H), 8.27 (d, J=8.4 Hz, 1H), 8.06-7.94 (m, 5H), 7.54-7.36 (m, 7H), 7.15 (dd, J=7.6, 1.6 Hz, 1H), 7.01 (br s, 2H), 6.39 (dd, J=7.6, 4.8 Hz, 1H), 3.78-3.68 (m, 2H), 3.64 (s, 2H), 3.38-3.35 (m, 2H), 2.56-2.52 (m, 2H), 2.47-2.40 (m, 2H).

### Example 146: 2-(4-(4-(2-(2-Aminopyridin-3-yl)-5-phenyl-3H-imidazo[4,5-b]pyridin-3-yl)benzyl)piperazine-1-carbonyl)nicotinonitrile

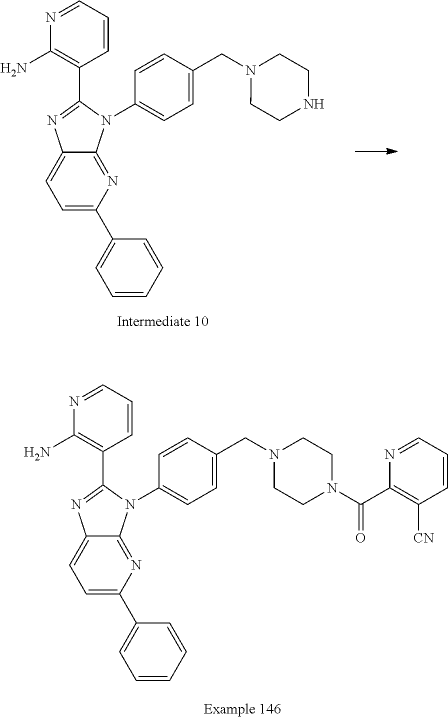 [SMILES: Nc1ncccc1-c1nc2ccc(-c3ccccc3)nc2n1-c1ccc(CN2CCNCC2)cc1 ; N#Cc1cccnc1C(=O)N1CCN(Cc2ccc(-n3c(-c4cccnc4N)nc4ccc(-c5ccccc5)nc43)cc2)CC1]

Following the general procedure of Example 138, the reaction of Intermediate 10 (150 mg, 325 μmol) with 3-cyanopicolinic acid (53 mg, 357 μmol) was carried out. After purified by prep-HPLC (column: Phenomenex C18 150*25 mm*10 μm; mobile phase: [water (NH₄HCO₃)-ACN]; B %: 35%-65%, 8 min), 2-(4-(4-(2-(2-aminopyridin-3-yl)-5-phenyl-3H-imidazo[4,5-b]pyridin-3-yl)benzyl)piperazine-1-carbonyl)nicotinonitrile (Example 146, 38.7 mg, yield: 20%) was obtained as a light-yellow solid. MS: m/z=592.2 [M+H]⁺. ¹H NMR (400 MHz, Dimethylsulfoxide-d₆) δ 8.86 (d, J=4.8 Hz, 1H), 8.47 (dd, J=8.0, 1.2 Hz, 1H), 8.27 (d, J=8.4 Hz, 1H), 8.08-7.93 (m, 4H), 7.70 (dd, J=8.0, 4.8 Hz, 1H), 7.53-7.42 (m, 6H), 7.42-7.36 (m, 1H), 7.14 (dd, J=7.6, 1.6 Hz, 1H), 7.02 (br s, 2H), 6.39 (dd, J=7.6, 4.8 Hz, 1H), 3.77-3.70 (m, 2H), 3.64 (s, 2H), 3.31-3.26 (m, 2H), 2.56-2.52 (m, 2H), 2.46-2.39 (m, 2H).

### Example 147: N-(4-(2-(2-Aminopyridin-3-yl)-3H-imidazo[4,5-b]pyridin-3-yl)benzyl)-4-cyanopicolinamide

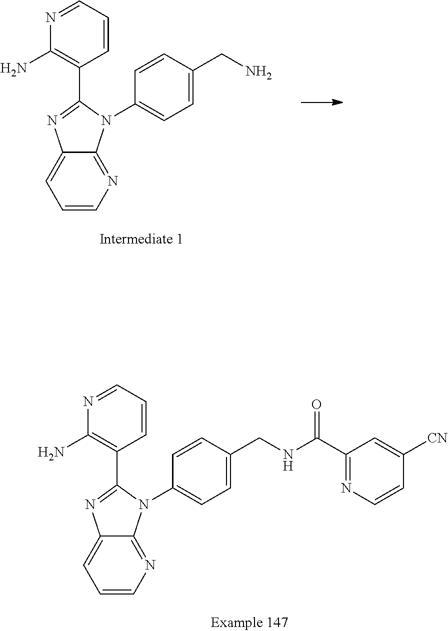 [SMILES: NCc1ccc(-n2c(-c3cccnc3N)nc3cccnc32)cc1 ; N#Cc1ccnc(C(=O)NCc2ccc(-n3c(-c4cccnc4N)nc4cccnc43)cc2)c1]

Following the general procedure of Example 138, the reaction of Intermediate 1 (200 mg, 632 μmol) with 4-cyanopyridine-2-carboxylic acid (93.6 mg, 632 μmol) was carried out. After purified by prep-HPLC (column: Phenomenex luna C₁₈ 150×25 mm×10 μm; mobile phase: [water (FA)-ACN]; B %: 7%-37%, 10 min), N-(4-(2-(2-aminopyridin-3-yl)-3H-imidazo[4,5-b]pyridin-3-yl)benzyl)-4-cyanopicolinamide (Example 147, 19.3 mg, yield: 7%) was obtained as a yellow solid. MS: m/z=447.3 [M+H]⁺. ¹H NMR (400 MHz, Dimethylsulfoxide-d₆) δ 9.64 (t, J=6.4 Hz, 1H), 8.93 (d, J=5.2 Hz, 1H), 8.38 (s, 1H), 8.29 (dd, J=4.8, 1.6 Hz, 1H), 8.19 (dd, J=8.0, 1.2 Hz, 1H), 8.11 (dd, J=4.8, 1.6 Hz, 1H), 7.98 (dd, J=4.8, 2.0 Hz, 1H), 7.46 (d, J=8.4 Hz, 2H), 7.39-7.36 (m, 3H), 7.22 (dd, J=7.6, 2.0 Hz, 1H), 6.96 (br s, 2H), 6.40 (dd, J=7.6, 4.8 Hz, 1H), 4.59 (d, J=6.4 Hz, 2H).

### Example 148: N-(4-(2-(2-Aminopyridin-3-yl)-3H-imidazo[4,5-b]pyridin-3-yl)benzyl)-2-cyanopyrimidine-4-carboxamide

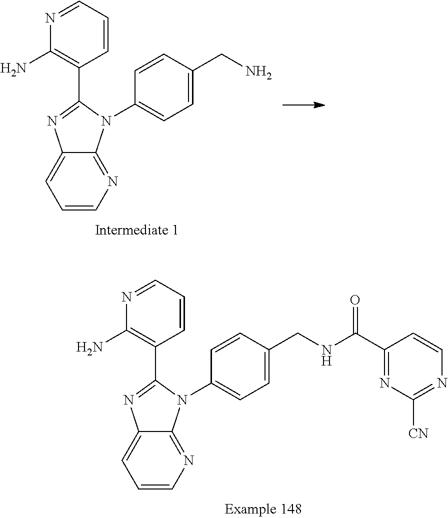 [SMILES: NCc1ccc(-n2c(-c3cccnc3N)nc3cccnc32)cc1 ; N#Cc1nccc(C(=O)NCc2ccc(-n3c(-c4cccnc4N)nc4cccnc43)cc2)n1]

Following the general procedure of Example 138, the reaction of Intermediate 1 (100 mg, 316 μmol) with 2-cyanopyrimidine-4-carboxylic acid (47.1 mg, 316 μmol) was carried out. After purified by prep-HPLC (column: Waters xbridge 150×25 mm 10 μm; mobile phase: [water (NH₄HCO₃)-ACN]; B %: 19%-49%, 8 min), N-(4-(2-(2-aminopyridin-3-yl)-3H-imidazo[4,5-b]pyridin-3-yl)benzyl)-2-cyanopyrimidine-4-carboxamide (Example 148, 8 mg, yield: 5%) was obtained as a white solid. MS: m/z=448.1 [M+H]⁺. ¹H NMR (400 MHz, Chloroform-d)) δ 9.13 (d, J=5.2 Hz, 1H), 8.41 (dd, J=4.8, 1.2 Hz, 1H), 8.37 (d, J=5.2 Hz, 1H), 8.28-8.22 (m, 1H), 8.12 (dd, J=8.0, 1.6 Hz, 1H), 8.02 (dd, J=5.2, 1.6 Hz, 1H), 7.56 (d, J=8.4 Hz, 2H), 7.41 (d, J=8.4 Hz, 2H), 7.36-7.27 (m, 3H), 7.21 (dd, J=7.6, 1.6 Hz, 1H), 6.45 (dd, J=8.0, 5.2 Hz, 1H), 4.80 (d, J=6.4 Hz, 2H).

### Example 149: 2-(4-(4-(2-(2-Aminopyridin-3-yl)-3H-imidazo[4,5-b]pyridin-3-yl)benzyl)piperazine-1-carbonyl)isonicotinonitrile

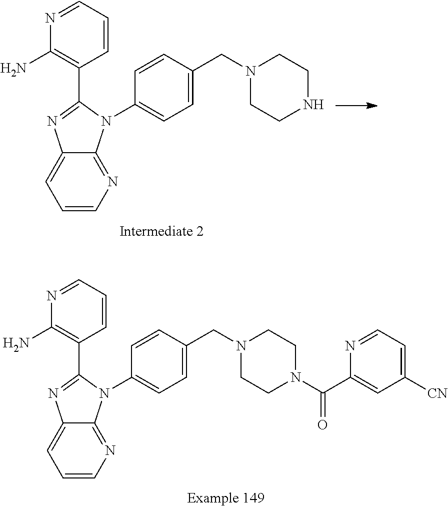 [SMILES: Nc1ncccc1-c1nc2cccnc2n1-c1ccc(CN2CCNCC2)cc1 ; N#Cc1ccnc(C(=O)N2CCN(Cc3ccc(-n4c(-c5cccnc5N)nc5cccnc54)cc3)CC2)c1]

Following the general procedure of Example 138, the reaction of Intermediate 2 (200 mg, 519 μmol) with 4-cyanopyridine-2-carboxylic acid (76.8 mg, 519 μmol) was carried out. After purified by prep-HPLC (column: Waters Xbridge 150×25 mm×5 μm; mobile phase: [water (NH₄HCO₃)-ACN]; B %: 17%-47%, 8 min), 2-(4-(4-(2-(2-aminopyridin-3-yl)-3H-imidazo[4,5-b]pyridin-3-yl)benzyl)piperazine-1-carbonyl)isonicotinonitrile (Example 149, 31.1 mg, yield: 11%) was obtained as a yellow solid. MS: m/z=516.1 [M+H]⁺. ¹H NMR (400 MHz, Dimethylsulfoxide-d₆) δ 8.76 (d, J=4.8 Hz, 1H), 8.40 (dd, J=4.8, 1.2 Hz, 1H), 8.10 (dd, J=8.0, 1.2 Hz, 1H), 8.05 (dd, J=4.8, 1.6 Hz, 1H), 7.94 (s, 1H), 7.57 (dd, J=5.2, 1.6 Hz, 1H), 7.51 (d, J=8.4 Hz, 2H), 7.36-7.30 (m, 3H), 7.07 (dd, J=8.0, 1.6 Hz, 1H), 6.62 (br s, 2H), 6.33 (dd, J=7.6, 4.8 Hz, 1H), 3.88-3.84 (m, 2H), 3.67-3.67 (m, 4H), 2.63 (t, J=4.8 Hz, 2H), 2.55-2.49 (m, 2H).

### Example 150: N-(1-(4-(2-(2-Aminopyridin-3-yl)-5-phenyl-3H-imidazo[4,5-b]pyridin-3-yl)benzyl)piperidin-4-yl)-4-cyanopicolinamide

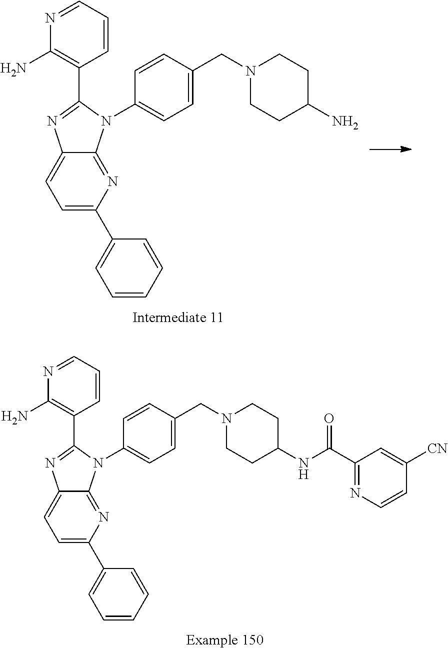 [SMILES: Nc1ncccc1-c1nc2ccc(-c3ccccc3)nc2n1-c1ccc(CN2CCC(N)CC2)cc1 ; N#Cc1ccnc(C(=O)NC2CCN(Cc3ccc(-n4c(-c5cccnc5N)nc5ccc(-c6ccccc6)nc54)cc3)CC2)c1]

To a solution of Intermediate 11 (150 mg, 315 μmol) and 4-cyanopyridine-2-carboxylic acid (90.7 mg, 473 μmol) in CH₂Cl₂ (2 mL) was added EDCI (90.7 mg, 473 μmol), HOBt (63.9 mg, 473 μmol) and DIEA (163 mg, 1.26 mmol). The resulting mixture was stirred at 25° C. for 16 hr. Then the reaction mixture was diluted with 5 mL H₂O and extracted with EtOAc (5 mL×2). The combined organic layers were dried over Na₂SO₄, filtered and concentrated under reduced pressure to give a residue. The residue was purified by prep-HPLC(column: Waters xbridge 150×25 mm 10 μm; mobile phase: [water (NH₄HCO₃)-ACN]; B %: 41%-71%, 8 min), N-(1-(4-(2-(2-aminopyridin-3-yl)-5-phenyl-3H-imidazo[4,5-b]pyridin-3-yl)benzyl)piperidin-4-yl)-4-cyanopicolinamide (Example 150, 30 mg, yield: 15%) was obtained as a yellow solid. MS: m/z=606.1 [M+H]⁺. ¹H NMR (400 MHz, Chloroform-d) δ 8.75 (d, J=5.2 Hz, 1H), 8.41 (s, 1H), 8.14 (d, J=8.4 Hz, 1H), 8.06 (dd, J=4.8, 1.6 Hz, 1H), 8.01 (d, J=7.6 Hz, 2H), 7.99-7.93 (m, 1H), 7.81 (d, J=8.4 Hz, 1H), 7.70-7.60 (m, 3H), 7.55-7.41 (m, 5H), 7.40-7.35 (m, 1H), 7.09 (dd, J=7.6, 1.6 Hz, 1H), 6.67 (br s, 1H), 6.37 (dd, J=7.6, 4.8 Hz, 1H), 4.19-4.09 (m, 1H), 3.95-3.69 (m, 2H), 3.30-3.04 (m, 2H), 2.65-2.46 (m, 2H), 2.22-2.03 (m, 4H).

### Example 151: N-(1-(4-(2-(2-Aminopyridin-3-yl)-5-phenyl-3H-imidazo[4,5-b]pyridin-3-yl)benzyl)piperidin-4-yl)-2-cyanopyrimidine-4-carboxamide

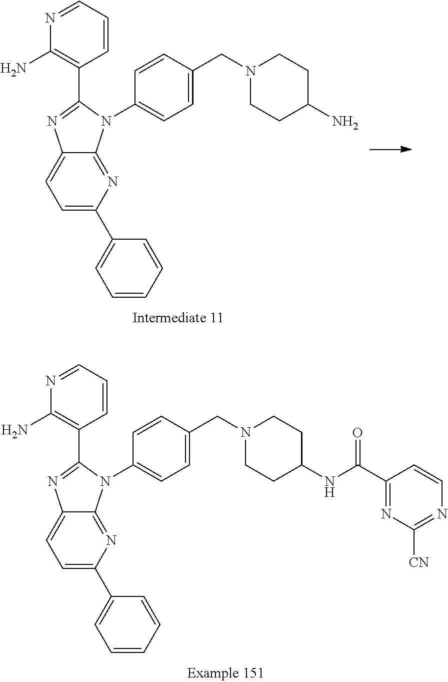 [SMILES: Nc1ncccc1-c1nc2ccc(-c3ccccc3)nc2n1-c1ccc(CN2CCC(N)CC2)cc1 ; N#Cc1nccc(C(=O)NC2CCN(Cc3ccc(-n4c(-c5cccnc5N)nc5ccc(-c6ccccc6)nc54)cc3)CC2)n1]

Following the general procedure of Example 150, the reaction of Intermediate 11 (150 mg, 315 μmol) with 2-cyanopyrimidine-4-carboxylic acid (47 mg, 315 μmol) was carried out. After purified by prep-HPLC (column: Waters xbridge 150×25 mm 10 μm; mobile phase: [water (NH₄HCO₃)-ACN]; B %: 41%-71%, 8 min), N-(1-(4-(2-(2-aminopyridin-3-yl)-5-phenyl-3H-imidazo[4,5-b]pyridin-3-yl)benzyl)piperidin-4-yl)-2-cyanopyrimidine-4-carboxamide (Example 151, 35.2 mg, yield: 18%) was obtained as a yellow solid. MS: m/z=607.1 [M+H]⁺. ¹H NMR (400 MHz, Chloroform-d)) δ 9.08 (d, J=5.2 Hz, 1H), 8.32 (d, J=5.2 Hz, 1H), 8.13 (d, J=8.4 Hz, 1H), 8.11-8.04 (m, 1H), 8.02 (d, J=7.6 Hz, 1H), 7.80 (d, J=8.4 Hz, 1H), 7.74 (d, J=8.0 Hz, 1H), 7.51 (d, J=8.4 Hz, 2H), 7.46-7.40 (m, 4H), 7.39-7.35 (m, 1H), 7.10 (d, J=7.6 Hz, 1H), 6.61 (br s, 2H), 6.36 (dd, J=7.6, 4.8 Hz, 1H), 4.12-3.96 (m, 1H), 3.65 (s, 2H), 2.95 (d, J=11.6 Hz, 2H), 2.34-2.25 (m, 2H), 2.11-2.01 (m, 2H), 1.82-1.73 (m, 2H).

### Example 152: 4-(4-(4-(2-(2-aminopyridin-3-yl)-3H-imidazo[4,5-b]pyridin-3-yl)benzyl)piperazine-1-carbonyl)pyrimidine-2-carbonitrile

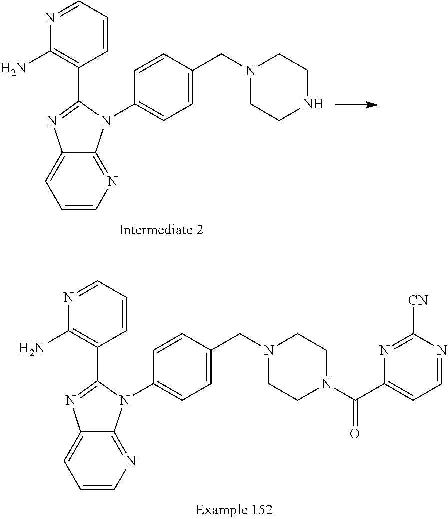 [SMILES: Nc1ncccc1-c1nc2cccnc2n1-c1ccc(CN2CCNCC2)cc1 ; N#Cc1nccc(C(=O)N2CCN(Cc3ccc(-n4c(-c5cccnc5N)nc5cccnc54)cc3)CC2)n1]

Following the general procedure Example 150, the reaction of Intermediate 2 (130 mg, 337 μmol) with 2-cyanopyrimidine-4-carboxylic acid (67.1 mg, 405 μmol) was carried out. After purified by prep-HPLC (column: Phenomenex C18 150×25 mm 10 μm; mobile phase: [water (NH₄HCO₃)-ACN]; B %: 18%-48%, 8 min), 4-(4-(4-(2-(2-aminopyridin-3-yl)-3H-imidazo[4,5-b]pyridin-3-yl)benzyl)piperazine-1-carbonyl)pyrimidine-2-carbonitrile (Example 152, 20 mg, yield: 11%) was obtained as a yellow solid. MS: m/z=517.2 [M+H]⁺. ¹H NMR (400 MHz, Dimethylsulfoxide-d₆) δ 9.15 (d, J=5.2 Hz, 1H), 8.33 (dd, J=4.8, 1.2 Hz, 1H), 8.20 (dd, J=8.0, 1.2 Hz, 1H), 8.01-7.96 (m, 2H), 7.49-7.45 (m, 2H), 7.41-7.36 (m, 3H), 7.16 (dd, J=7.6, 1.6 Hz, 1H), 7.00 (br s, 2H), 6.38 (dd, J=7.6, 4.8 Hz, 1H), 3.71-3.66 (m, 2H), 3.62 (s, 2H), 3.41-3.38 (m, 2H), 2.59-2.55 (m, 2H), 2.43-2.39 (m, 2H).

### Example 153: 3-(4-(4-(2-(2-Aminopyridin-3-yl)-3H-imidazo[4,5-b]pyridin-3-yl)benzyl)piperazine-1-carbonyl)picolinonitrile

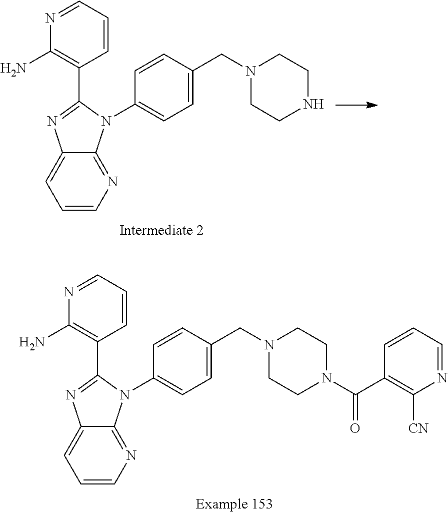 [SMILES: Nc1ncccc1-c1nc2cccnc2n1-c1ccc(CN2CCNCC2)cc1 ; N#Cc1ncccc1C(=O)N1CCN(Cc2ccc(-n3c(-c4cccnc4N)nc4cccnc43)cc2)CC1]

Following the general procedure of Example 150, the reaction of Intermediate 2 (200 mg, 518 μmol) with 2-cyanonicotinic acid (115 mg, 778 μmol) was carried out. After purified by prep-HPLC (column: Phenomenex C18 150×25 mm 10 μm; mobile phase: [water (NH₄HCO₃)-ACN]; B %: 16%-46%, 8 min), 33-(4-(4-(2-(2-aminopyridin-3-yl)-3H-imidazo[4,5-b]pyridin-3-yl)benzyl)piperazine-1-carbonyl)picolinonitrile (Example 153, 11.8 mg, yield: 4.4%) was obtained as a light-yellow powder. MS: m/z=516.4 [M+H]⁺. ¹H NMR (400 MHz, Dimethylsulfoxide-d₆) δ 8.82 (dd, J=4.8, 1.6 Hz, 1H), 8.31 (dd, J=4.8, 1.6 Hz, 1H), 8.20 (dd, J=8.0, 1.2 Hz, 1H), 8.09 (dd, J=8.0, 1.6 Hz, 1H), 7.98 (dd, J=4.8, 2.0 Hz, 1H), 7.83 (dd, J=8.0, 4.8 Hz, 1H), 7.47 (d, J=8.4 Hz, 2H), 7.41-7.36 (m, 3H), 7.16 (dd, J=7.6, 1.6 Hz, 11H), 6.99 (br s, 2H), 6.38 (dd, J=7.6, 4.8 Hz, JH), 3.78-3.71 (m, 2H), 3.61 (s, 2H), 3.32-3.28 (m, 2H), 2.56-2.51 (m, 2H), 2.45-2.45 (m, 2H).

### Example 154: 4-(4-(4-(2-(2-Aminopyridin-3-yl)-3H-imidazo[4,5-b]pyridin-3-yl)benzyl)piperazine-1-carbonyl)nicotinonitrile

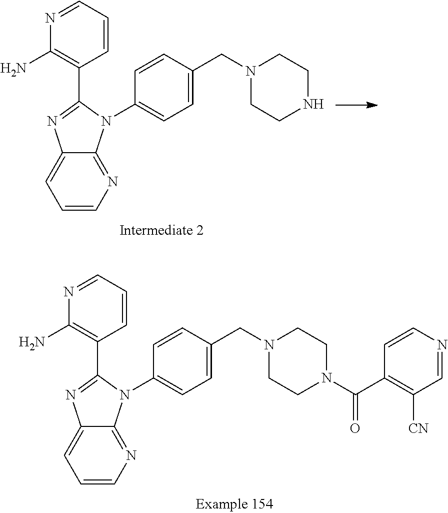 [SMILES: Nc1ncccc1-c1nc2cccnc2n1-c1ccc(CN2CCNCC2)cc1 ; N#Cc1cnccc1C(=O)N1CCN(Cc2ccc(-n3c(-c4cccnc4N)nc4cccnc43)cc2)CC1]

Following the general procedure of Example 150, the reaction of Intermediate 2 (300 mg, 778 μmol) with 3-cyanoisonicotinic acid (172 mg, 1.17 mmol) was carried out. After purified by silica gel flash chromatography (Eluent of 0˜8% EtOAc in petroleum ether), then by prep-TLC (CH₂Cl₂:MeOH=10:1), 4-(4-(4-(2-(2-aminopyridin-3-yl)-3H-imidazo[4,5-b]pyridin-3-yl)benzyl)piperazine-1-carbonyl)nicotinonitrile (Example 154, 34 mg, yield: 7.9%) was obtained as a light-yellow powder. MS: m/z=516.3 [M+H]⁺. ¹H NMR (400 MHz, Dimethylsulfoxide-d₆) δ 9.14 (s, 1H), 8.93 (d, J=4.8 Hz, 1H), 8.31 (dd, J=4.8, 3.6 Hz, 1H), 8.20 (dd, J=8.0, 1.2 Hz, 1H), 7.98 (dd, J=4.8, 1.6 Hz, 1H), 7.65 (d, J=4.8 Hz, 1H), 7.47 (d, J=8.4 Hz, 2H), 7.41-7.37 (m, 3H), 7.16 (dd, J=7.6, 1.6 Hz, 1H), 6.99 (br s, 2H), 6.38 (dd, J=7.6, 4.8 Hz, 1H), 3.73-3.68 (m, 2H), 3.61 (s, 2H), 3.27-3.25 (m, 2H), 2.54-2.52 (m, 2H), 2.42-2.39 (m, 2H).

### Example 155: 3-(4-(4-(2-(2-Aminopyridin-3-yl)-3H-imidazo[4,5-b]pyridin-3-yl)benzyl)piperazine-1-carbonyl)isonicotinonitrile

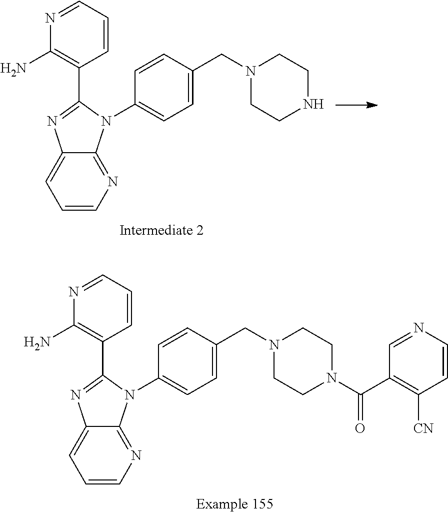 [SMILES: Nc1ncccc1-c1nc2cccnc2n1-c1ccc(CN2CCNCC2)cc1 ; N#Cc1ccncc1C(=O)N1CCN(Cc2ccc(-n3c(-c4cccnc4N)nc4cccnc43)cc2)CC1]

Following the general procedure of Example 150, the reaction of Intermediate 2 (200 mg, 518 μmol) with 4-cyanonicotinic acid (115 mg, 778 μmol) was carried out. After purified by prep-HPLC (column: Phenomenex C18 150×25 mm 10 μm; mobile phase: [water (NH₄HCO₃)-ACN]; B %: 16%-46%, 8 min), 3-(4-(4-(2-(2-aminopyridin-3-yl)-3H-imidazo[4,5-b]pyridin-3-yl)benzyl)piperazine-1-carbonyl)isonicotinonitrile (Example 155, 12.1 mg, yield: 4.4%) was obtained as a light-yellow powder. MS: m/z=516.2 [M+H]⁺. ¹H NMR (400 MHz, Dimethylsulfoxide-d₆) δ 8.90 (d, J=4.8 Hz, 1H), 8.86 (s, 1H), 8.31 (dd, J=4.8, 1.2 Hz, 1H), 8.20 (dd, J=8.0, 1.6 Hz, 1H), 8.01-7.96 (m, 2H), 7.47 (d, J=8.0 Hz, 2H), 7.47-7.35 (m, 3H), 7.16 (dd, J=7.6, 1.6 Hz, 1H), 6.99 (br s, 2H), 6.38 (dd, J=7.6, 4.8 Hz, 1H), 3.76-3.70 (m, 2H), 3.61 (s, 2H), 3.33-3.29 (m, 2H), 2.55-2.51 (m, 2H), 2.45-2.39 (m, 2H).

### Example 156: 4-((1-(4-(2-(2-Aminopyridin-3-yl)-5-phenyl-3H-imidazo[4,5-b]pyridin-3-yl)benzyl)piperidin-4-yl)(methyl)amino)pyrimidine-2-carbonitrile

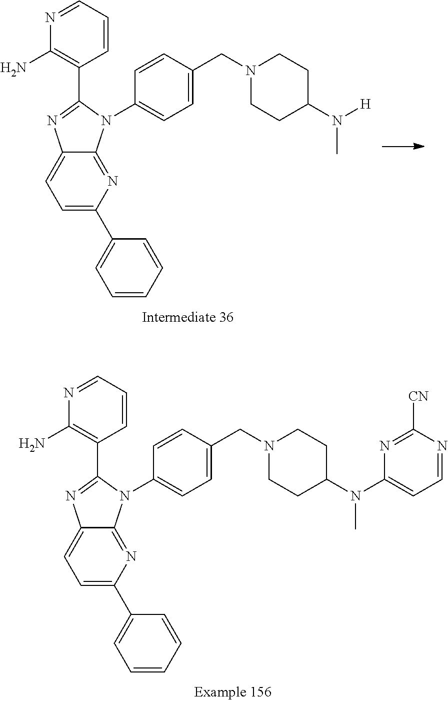 [SMILES: CNC1CCN(Cc2ccc(-n3c(-c4cccnc4N)nc4ccc(-c5ccccc5)nc43)cc2)CC1 ; CN(c1ccnc(C#N)n1)C1CCN(Cc2ccc(-n3c(-c4cccnc4N)nc4ccc(-c5ccccc5)nc43)cc2)CC1]

To a solution of Intermediate 36 (130 mg, 266 μmol) in DMF (2 mL) was added 4-chloropyrimidine-2-carbonitrile (44 mg, 319 μmol), NaI (3.98 mg, 27 μmol) and K₂CO₃ (110 mg, 797 μmol). The mixture was stirred at 80° C. for 2 hr. The reaction mixture was poured into H₂O (10 mL), extracted with EtOAc (15 mL). The organic phase was dried over anhydrous Na₂SO₄, filtered, and concentrated under reduced pressure to give a residue. The residue was purified by silica gel flash chromatography (Eluent of 0˜63% EtOAc in petroleum ether), 4-((1-(4-(2-(2-aminopyridin-3-yl)-5-phenyl-3H-imidazo[4,5-b]pyridin-3-yl)benzyl)piperidin-4-yl)(methyl)amino)pyrimidine-2-carbonitrile (Example 156, 59.4 mg, yield: 37%) was obtained as a yellow solid. MS: m/z=593.4 [M+H]⁺. ¹H NMR (400 MHz, Methanol-d₄) δ 8.18-8.16 (m, 2H), 8.02-8.00 (m, 2H), 7.97 (dd, J=5.2, 2.0 Hz, 1H), 7.93 (d, J=8.8 Hz, 1H), 7.56 (d, J=8.4 Hz, 2H), 7.48-7.30 (m, 6H), 6.89-6.76 (m, 1H), 6.47 (dd, J=7.6, 5.2 Hz, 1H), 3.75 (s, 2H), 3.35-3.31 (m, 1H), 3.14-3.11 (m, 2H), 2.98 (s, 3H), 2.39-2.33 (m, 2H), 1.98-1.87 (m, 2H), 1.72-1.69 (m, 2H).

### Example 157: 5-((1-(4-(2-(2-aminopyridin-3-yl)-3H-imidazo[4,5-b]pyridin-3-yl)benzyl)piperidin-4-yl)amino)picolinonitrile

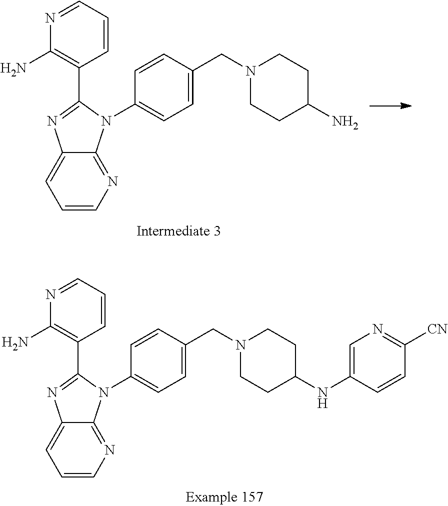 [SMILES: Nc1ncccc1-c1nc2cccnc2n1-c1ccc(CN2CCC(N)CC2)cc1 ; N#Cc1ccc(NC2CCN(Cc3ccc(-n4c(-c5cccnc5N)nc5cccnc54)cc3)CC2)cn1]

Following the general procedure of Example 84, the reaction of Intermediate 3 (180 mg, 451 μmol) with 5-fluoropicolinonitrile (110 mg, 901 μmol) was carried out. After purified by prep-HPLC (column: Phenomenex C18 150×25 mm 10 μm; mobile phase: [water (NH₄HCO₃)-ACN]; B %: 16%-46%, 8 min), 5-((1-(4-(2-(2-aminopyridin-3-yl)-3H-imidazo[4,5-b]pyridin-3-yl)benzyl)piperidin-4-yl)amino)picolinonitrile (Example 157, 20.6 mg, yield: 8.5%) was obtained as an off-white solid. MS: m/z=502.1 [M+H]⁺. ¹H NMR (400 MHz, Dimethylsulfoxide-d₆) δ 8.34 (dd, J=4.8, 1.2 Hz, 1H), 8.21 (dd, J=8.0, 1.2 Hz, 1H), 8.06 (d, J=2.8 Hz, 1H), 7.99 (dd, J=4.8, 1.6 Hz, 1H), 7.61 (d, J=8.8 Hz, 1H), 7.46 (d, J=8.4 Hz, 2H), 7.42-7.37 (m, 3H), 7.17 (dd, J=7.6, 1.6 Hz, 1H), 7.04-6.96 (m, 3H), 6.91 (d, J=7.6 Hz, 1H), 6.38 (dd, J=7.6, 4.8 Hz, 1H), 3.58 (s, 2H), 2.90-2.75 (m, 3H), 2.21-2.12 (m, 2H), 1.96-1.87 (m, 2H), 1.50-1.40 (m, 2H).

### Example 158: 5-((1-(4-(2-(2-Aminopyridin-3-yl)-5-phenyl-3H-imidazo[4,5-b]pyridin-3-yl)benzyl)piperidin-4-yl)amino)pyridazine-3-carbonitrile

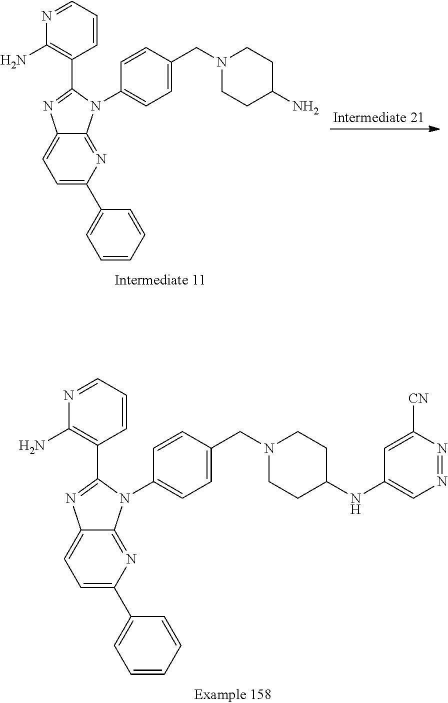 [SMILES: Nc1ncccc1-c1nc2ccc(-c3ccccc3)nc2n1-c1ccc(CN2CCC(N)CC2)cc1 ; N#Cc1cc(NC2CCN(Cc3ccc(-n4c(-c5cccnc5N)nc5ccc(-c6ccccc6)nc54)cc3)CC2)cnn1]

Following the general procedure of Example 47, the reaction of Intermediate 11 (200 mg, 420 μmol) with Intermediate 21 (58 mg, 420 μmol) was carried out. After purified by prep-HPLC (column: Waters xbridge 150*25 mm 10 μm; mobile phase: [water (NH₄HCO₃)-ACN]; B %: 37%-67%, 10 min), 5-((1-(4-(2-(2-aminopyridin-3-yl)-5-phenyl-3H-imidazo[4,5-b]pyridin-3-yl)benzyl)piperidin-4-yl)amino)pyridazine-3-carbonitrile (Example 158, 75.3 mg, yield: 31%) was obtained as a yellow solid. MS: m/z=579.4 [M+H]⁺. ¹H NMR (400 MHz, Dimethylsulfoxide-d₆) δ 8.71 (br s, 1H), 8.27 (d, J=8.4 Hz, 1H), 8.06-7.95 (m, 4H), 7.67-7.57 (m, 1H), 7.53-7.43 (m, 6H), 7.41-7.37 (m, 1H), 7.37-7.29 (m, 1H), 7.16 (dd, J=7.6, 1.6 Hz, 1H), 7.03 (br s, 2H), 6.38 (dd, J=7.6, 4.8 Hz, 1H), 3.60 (s, 2H), 3.51-3.37 (m, 1H), 2.83 (d, J=11.6 Hz, 2H), 2.22-2.12 (m, 2H), 1.95-1.85 (m, 2H), 1.52-1.40 (m, 2H).

### Example 159: 5-(4-(4-(2-(2-Aminopyridin-3-yl)-5-phenyl-3H-imidazo[4,5-b]pyridin-3-yl)benzyl)piperazin-1-yl)pyridazine-3-carbonitrile

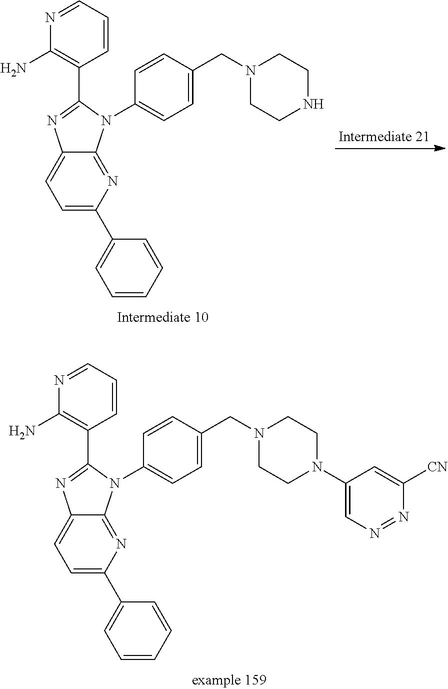 [SMILES: Nc1ncccc1-c1nc2ccc(-c3ccccc3)nc2n1-c1ccc(CN2CCNCC2)cc1 ; N#Cc1cc(N2CCN(Cc3ccc(-n4c(-c5cccnc5N)nc5ccc(-c6ccccc6)nc54)cc3)CC2)cnn1]

Following the general procedure of Example 47, the reaction of Intermediate 10 (200 mg, 433 μmol) with Intermediate 21 (60 mg, 433 μmol) was carried out. After purified by prep-HPLC (column: Waters xbridge 150*25 mm 10 μm; mobile phase: [water (NH₄HCO₃)-ACN]; B %: 35%-65%, 10 min), 5-(4-(4-(2-(2-aminopyridin-3-yl)-5-phenyl-3H-imidazo[4,5-b]pyridin-3-yl)benzyl)piperazin-1-yl)pyridazine-3-carbonitrile (Example 159, 48.7 mg, yield: 20%) was obtained as a yellow solid. MS: m/z=565.4 [M+H]⁺. ¹H NMR (400 MHz, Dimethylsulfoxide-d₆) δ 9.15 (d, J=3.2 Hz, 1H), 8.28 (d, J=8.4 Hz, 1H), 8.06-8.02 (m, 2H), 8.01-7.97 (m, 2H), 7.65 (d, J=3.2 Hz, 1H), 7.54-7.50 (m, 2H), 7.49-7.43 (m, 4H), 7.42-7.36 (m, 1H), 7.15 (dd, J=7.6, 1.6 Hz, 1H), 7.03 (br s, 2H), 6.36 (dd, J=7.6, 4.8 Hz, 1H), 3.65 (s, 2H), 3.61-3.53 (m, 4H), 2.56-2.50 (m, 4H).

### Example 160: N-(4-(2-(2-Aminopyridin-3-yl)-5-phenyl-3H-imidazo[4,5-b]pyridin-3-yl)benzyl)-6-cyanonicotinamide

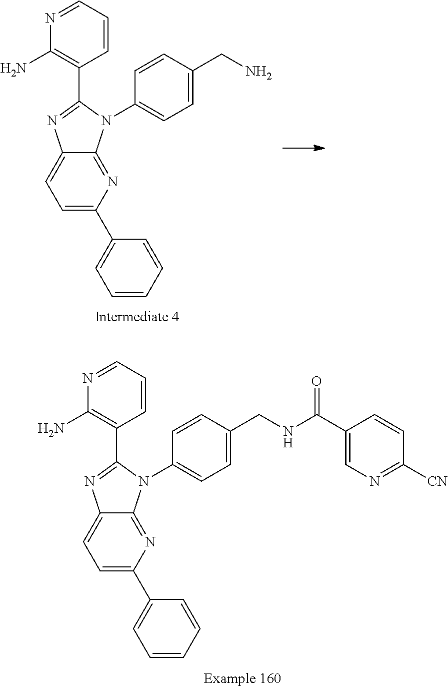 [SMILES: NCc1ccc(-n2c(-c3cccnc3N)nc3ccc(-c4ccccc4)nc32)cc1 ; N#Cc1ccc(C(=O)NCc2ccc(-n3c(-c4cccnc4N)nc4ccc(-c5ccccc5)nc43)cc2)cn1]

Following the general procedure of Example 23, the reaction of Intermediate 4 (200 mg, 510 μmol) with 6-cyanonicotinic acid (75 mg, 510 μmol) was carried out. After purified by prep-HPLC (column: Phenomenex luna C₁₈ 150×25 mm×10 μm; mobile phase: [water (FA)-ACN]; B %: 20%-50%, 10 min), N-(4-(2-(2-aminopyridin-3-yl)-5-phenyl-3H-imidazo[4,5-b]pyridin-3-yl)benzyl)-6-cyanonicotinamide (Example 160, 41.1 mg, yield: 15%) was obtained as a yellow solid. MS: m/z=523.2 [M+H]⁺. ¹H NMR (400 MHz, Dimethylsulfoxide-d₆) δ 9.56 (t, J=5.6 Hz, 1H), 9.20 (d, J=1.6 Hz, 1H), 8.48 (dd, J=2.4, 8.0 Hz, 1H), 8.26 (d, J=8.0 Hz, 1H), 8.20 (d, J=8.0 Hz, 1H), 8.04-7.97 (m, 4H), 7.54-7.50 (m, 2H), 7.49-7.47 (m, 2H), 7.47-7.44 (m, 2H), 7.42-7.37 (m, 1H), 7.22 (dd, J=1.6, 7.6 Hz, 1H), 6.92 (br s, 2H), 6.42 (dd, J=7.6, 4.8 Hz, 1H), 4.65 (d, J=5.6 Hz, 2H).

### Example 161: N-(4-(2-(2-Aminopyridin-3-yl)-5-phenyl-3H-imidazo[4,5-b]pyridin-3-yl)benzyl)-2-cyanoisonicotinamide

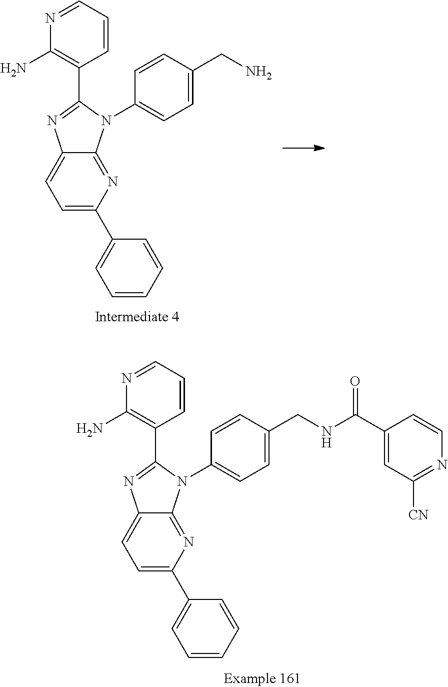 [SMILES: NCc1ccc(-n2c(-c3cccnc3N)nc3ccc(-c4ccccc4)nc32)cc1 ; N#Cc1cc(C(=O)NCc2ccc(-n3c(-c4cccnc4N)nc4ccc(-c5ccccc5)nc43)cc2)ccn1]

Following the general procedure of Example 23, the reaction of Intermediate 4 (150 mg, 382 μmol) with 2-cyanoisonicotinic acid (56.6 mg, 382 μmol) was carried out. After purified by prep-HPLC (column: Welch Xtimate C₁₈ 150×25 mm×5 μm; mobile phase: [water (NH₄HCO₃)-ACN]; B %: 33%-63%, 2 min), N-(4-(2-(2-aminopyridin-3-yl)-5-phenyl-3H-imidazo[4,5-b]pyridin-3-yl)benzyl)-2-cyanoisonicotinamide (Example 161, 30.2 mg, yield: 14%) was obtained as a yellow solid. MS: m/z=523.2 [M+H]⁺. ¹H NMR (400 MHz, Dimethylsulfoxide-d₆) δ 9.59 (t, J=5.6 Hz, 1H), 8.93 (d, J=4.8 Hz, 1H), 8.45 (s, 1H), 8.26 (d, J=8.4 Hz, 1H), 8.15 (dd, J=5.2, 1.6 Hz, 1H), 8.09-7.95 (m, 4H), 7.57-7.43 (m, 6H), 7.42-7.36 (m, 1H), 7.22 (dd, J=7.6, 2.0 Hz, 1H), 6.92 (br s, 2H), 6.42 (dd, J=7.6, 4.8 Hz, 1H), 4.65 (d, J=5.6 Hz, 2H).

### Example 162: N-(4-(2-(2-Aminopyridin-3-yl)-5-phenyl-3H-imidazo[4,5-b]pyridin-3-yl)benzyl)-6-cyanopicolinamide

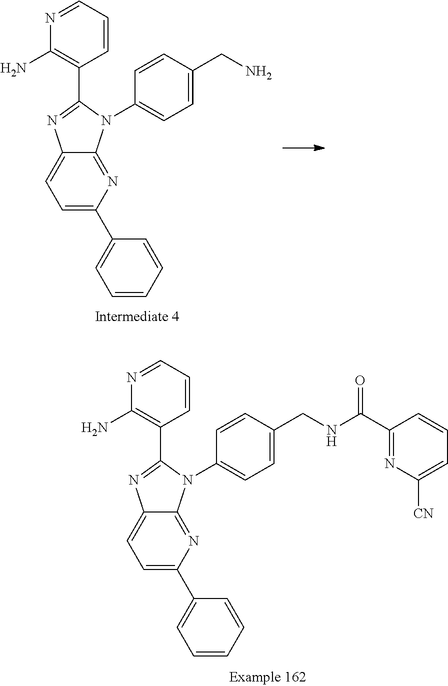 [SMILES: NCc1ccc(-n2c(-c3cccnc3N)nc3ccc(-c4ccccc4)nc32)cc1 ; N#Cc1cccc(C(=O)NCc2ccc(-n3c(-c4cccnc4N)nc4ccc(-c5ccccc5)nc43)cc2)n1]

Following the general procedure of Example 23, the reaction of Intermediate 4 (150 mg, 382 μmol) with 6-cyanopicolinic acid (56.6 mg, 382 μmol) was carried out. After purified by prep-HPLC (column: Waters xbridge 150×25 mm 10 μm; mobile phase: [water (NH₄HCO₃)-ACN]; B %: 36%-66%, 8 min), N-(4-(2-(2-aminopyridin-3-yl)-5-phenyl-3H-imidazo[4,5-b]pyridin-3-yl)benzyl)-6-cyanopicolinamide (Example 162, 41.2 mg, yield: 20%) was obtained as a yellow solid. MS: m/z=523.2 [M+H]⁺. ¹H NMR (400 MHz, Dimethylsulfoxide-d₆) δ 9.62 (t, J=6.4 Hz, 1H), 8.36-8.32 (m, 1H), 8.28-8.23 (m, 3H), 8.04-7.95 (m, 4H), 7.52-7.43 (m, 6H), 7.41-7.35 (m, 1H), 7.21 (dd, J=7.6, 1.6 Hz, 1H), 6.93 (br s, 2H), 6.42 (dd, J=7.6, 4.8 Hz, 1H), 4.62 (d, J=6.4 Hz, 2H).

### Example 163: 3-(4-(4-(2-(2-aminopyridin-3-yl)-5-phenyl-3H-imidazo[4,5-b]pyridin-3-yl)benzyl)piperazine-1-carbonyl)pyrazine-2-carbonitrile

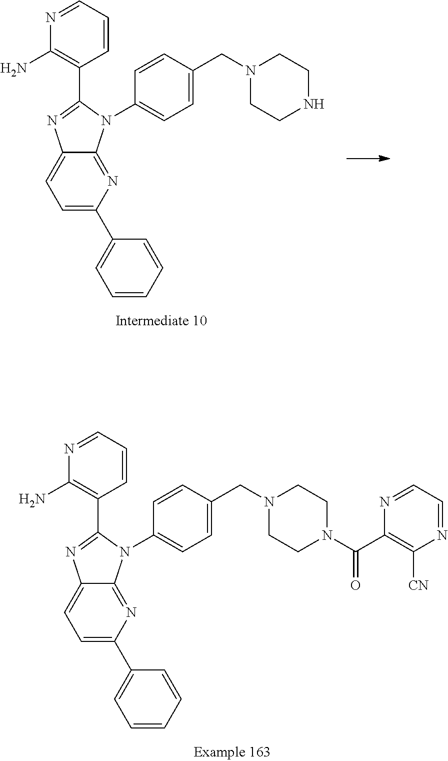 [SMILES: Nc1ncccc1-c1nc2ccc(-c3ccccc3)nc2n1-c1ccc(CN2CCNCC2)cc1 ; N#Cc1nccnc1C(=O)N1CCN(Cc2ccc(-n3c(-c4cccnc4N)nc4ccc(-c5ccccc5)nc43)cc2)CC1]

Following the general procedure of Example 23, the reaction of Intermediate 10 (250 mg, 812 μmol) with 3-carbamoylpyrazine-2-carboxylic acid (136 mg, 812 μmol) was carried out. After purified by prep-HPLC (column: Phenomenex C18 250*50 mm*10 μm; mobile phase: [water (NH₄HCO₃)-ACN]; B %: 30%-60%, 8 min), 3-(4-(4-(2-(2-aminopyridin-3-yl)-5-phenyl-3H-imidazo[4,5-b]pyridin-3-yl)benzyl)piperazine-1-carbonyl)pyrazine-2-carbonitrile (Example 163, 100 mg, yield: 28%) was obtained as a yellow lyophilized powder. MS: m/z=593.3 [M+H]⁺. ¹H NMR (400 MHz, Dimethylsulfoxide-d₆) δ 8.96 (dd, J=11.2, 2.4 Hz, 2H), 8.27 (d, J=8.4 Hz, 1H), 8.06-7.95 (m, 4H), 7.54-7.43 (m, 6H), 7.42-7.37 (m, 1H), 7.18-7.11 (m, 1H), 7.02 (br s, 2H), 6.36 (dd, J=7.6, 4.8 Hz, 1H), 3.79-3.73 (m, 2H), 3.65 (s, 2H), 3.45-3.39 (m, 2H), 2.58-2.52 (m, 2H), 2.45-2.42 (m, 2H).

### Example 164: 3-(4-(4-(2-(2-Aminopyridin-3-yl)-3H-imidazo[4,5-b]pyridin-3-yl)benzyl)piperazine-1-carbonyl)pyrazine-2-carbonitrile

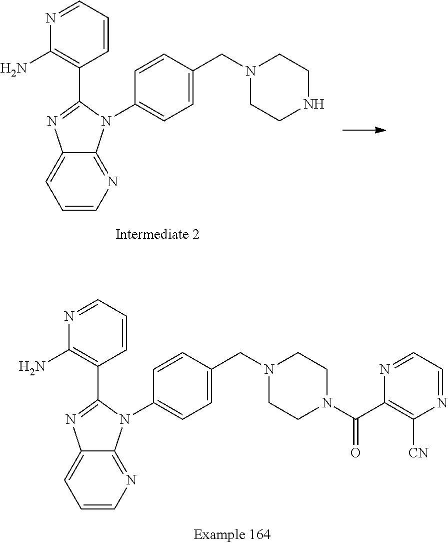 [SMILES: Nc1ncccc1-c1nc2cccnc2n1-c1ccc(CN2CCNCC2)cc1 ; N#Cc1nccnc1C(=O)N1CCN(Cc2ccc(-n3c(-c4cccnc4N)nc4cccnc43)cc2)CC1]

To a solution of 3-carbamoylpyrazine-2-carboxylic acid (156 mg, 934 μmol) in DMF (1 mL) was added DIEA (402 mg, 3.11 mmol), EDCI (224 mg, 1.17 mmol), HOBt (158 mg, 1.17 mmol) and Intermediate 2 (300 mg, 778 μmol). The mixture was stirred at 25° C. for 16 hr to give an orange liquid, which was filtered and concentrated under reduced pressure to give a residue. The residue was purified by reversed-phase HPLC (neutral condition: column: Phenomenex C18 150*25 mm*10 μm; mobile phase: (water-ACN); B %: 20%-50%, 15 min), 3-(4-(4-(2-(2-aminopyridin-3-yl)-3H-imidazo[4,5-b]pyridin-3-yl)benzyl)piperazine-1-carbonyl)pyrazine-2-carbonitrile (Example 164, 8.0 mg, yield: 1.9%) was obtained as a yellow powder. MS: m/z=517.3 [M+H]⁺. ¹H NMR (400 MHz, Methanol-d) δ 8.90-8.82 (m, 2H), 8.34 (d, J=4.8 Hz, 1H), 8.20 (dd, J=8.0, 1.2 Hz, 1H), 7.97 (dd, J=5.2, 1.6 Hz, 1H), 7.56 (d, J=8.4 Hz, 2H), 7.47-7.37 (m, 3H), 7.33 (dd, J=7.6, 1.6 Hz, 1H), 6.47 (dd, J=7.6, 5.2 Hz, 1H), 3.93-3.85 (m, 2H), 3.72 (s, 2H), 3.51-3.44 (m, 2H), 2.69 (t, J=4.8 Hz, 2H), 2.62-2.54 (m, 2H).

### Example 165: N-(1-(4-(2-(2-aminopyridin-3-yl)-6-phenyl-3H-imidazo[4,5-b]pyridin-3-yl)benzyl)piperidin-4-yl)-6-cyanonicotinamide

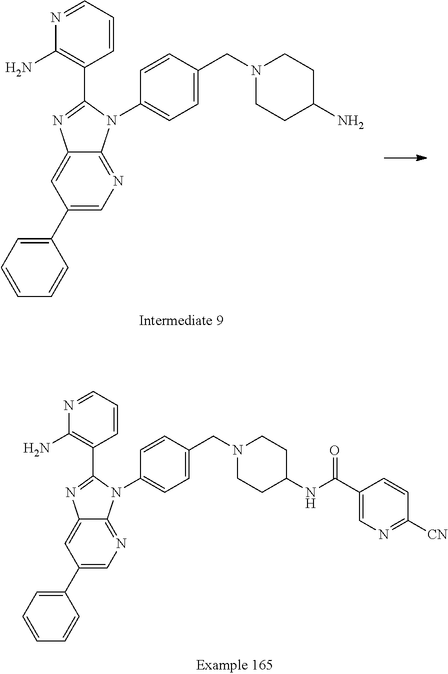 [SMILES: Nc1ncccc1-c1nc2cc(-c3ccccc3)cnc2n1-c1ccc(CN2CCC(N)CC2)cc1 ; N#Cc1ccc(C(=O)NC2CCN(Cc3ccc(-n4c(-c5cccnc5N)nc5cc(-c6ccccc6)cnc54)cc3)CC2)cn1]

Following the general procedure of Example 23, the reaction of Intermediate 9 (250 mg, 526 μmol) with 6-cyanopyridine-3-carboxylic acid (85.7 mg, 578 μmol) was carried out. After prep-HPLC (column: Waters xbridge 150*25 mm 10 μm; mobile phase: [water (NH₄HCO₃)-ACN]; B %: 19%-49%, 9 min), N-(1-(4-(2-(2-aminopyridin-3-yl)-6-phenyl-3H-imidazo[4,5-b]pyridin-3-yl)benzyl)piperidin-4-yl)-6-cyanonicotinamide (Example 165, 82 mg, yield: 25%) was obtained as a light-yellow solid. MS: m/z=606.2 [M+H]⁺. ¹H NMR (400 MHz, Dimethylsulfoxide-d₆) δ 9.11 (d, J=1.6 Hz, 1H), 8.72 (d, J=7.6 Hz, 1H), 8.64 (d, J=2.0 Hz, 1H), 8.47 (d, J=2.0 Hz, 1H), 8.41-8.38 (m, 1H), 8.17 (d, J=8.0 Hz, 1H), 8.00 (dd, J=4.8, 1.6 Hz, 1H), 7.78 (d, J=7.2 Hz, 2H), 7.54-7.47 (m, 4H), 7.43-7.40 (m, 3H), 7.2 (dd, J=8, 1.6 Hz, 1H), 7.07 (br s, 2H), 6.39 (dd, J=8, 4.8 Hz, 1H), 3.85-3.78 (m, 1H), 3.58 (s, 2H), 2.87 (d, J=11.2 Hz, 2H), 2.1 (t, J=11.2 Hz, 2H), 1.85 (d, J=10.0 Hz, 2H), 1.65-1.57 (m, 2H).

### Example 166: 5-(4-(4-(2-(2-Aminopyridin-3-yl)-5-phenyl-3H-imidazo[4,5-b]pyridin-3-yl)benzyl)piperazine-1-carbonyl)picolinonitrile

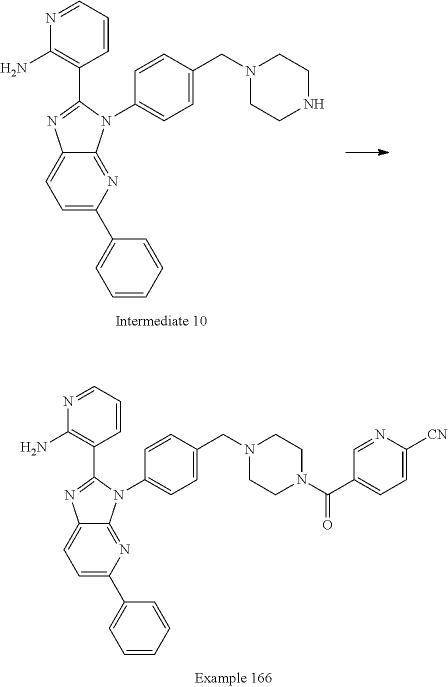 [SMILES: Nc1ncccc1-c1nc2ccc(-c3ccccc3)nc2n1-c1ccc(CN2CCNCC2)cc1 ; N#Cc1ccc(C(=O)N2CCN(Cc3ccc(-n4c(-c5cccnc5N)nc5ccc(-c6ccccc6)nc54)cc3)CC2)cn1]

Following the general procedure of Example 138, the reaction of Intermediate 10 (150 mg, 325 μmol) with 6-cyanonicotinic acid (53 mg, 357 μmol) was carried out. After purified by prep-HPLC (column: Waters xbridge 150*25 mm 10 μm; mobile phase: [water (NH₄HCO₃)-ACN]; B %: 42%-72%, 9 min), 5-(4-(4-(2-(2-aminopyridin-3-yl)-5-phenyl-3H-imidazo[4,5-b]pyridin-3-yl)benzyl)piperazine-1-carbonyl)picolinonitrile (Example 166, 27.5 mg, yield: 14%) was obtained as a yellow solid. MS: m/z=592.2 [M+H]⁺. ¹H NMR (400 MHz, Chloroform-d) δ 8.76 (s, 1H), 8.14 (d, J=8.4 Hz, 1H), 8.08-7.98 (m, 3H), 7.91 (dd, J=7.6, 2.0 Hz, 1H), 7.83-7.75 (m, 2H), 7.51-7.36 (m, 7H), 7.11 (d, J=7.6 Hz, 1H), 6.94-6.64 (m, 2H), 6.40-6.33 (m, 1H), 3.93-3.79 (m, 2H), 3.66 (s, 2H), 3.48-3.39 (m, 2H), 2.67-2.58 (m, 2H), 2.55-2.46 (m, 2H).

### Example 167: N-(1-(4-(2-(2-Aminopyridin-3-yl)-5-phenyl-3H-imidazo[4,5-b]pyridin-3-yl)benzyl)piperidin-4-yl)-6-cyanonicotinamide

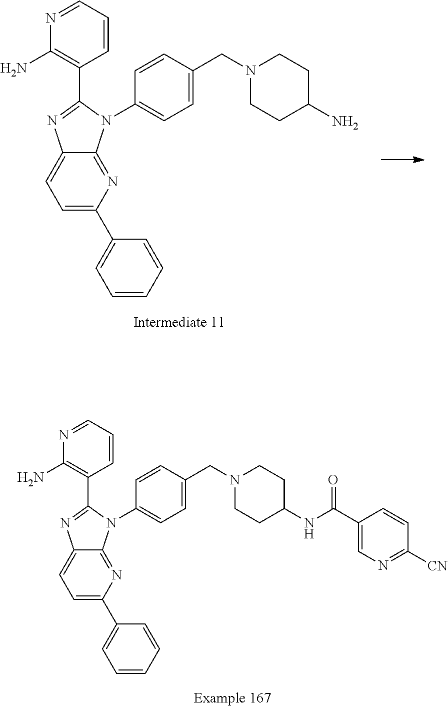 [SMILES: Nc1ncccc1-c1nc2ccc(-c3ccccc3)nc2n1-c1ccc(CN2CCC(N)CC2)cc1 ; N#Cc1ccc(C(=O)NC2CCN(Cc3ccc(-n4c(-c5cccnc5N)nc5ccc(-c6ccccc6)nc54)cc3)CC2)cn1]

Following the general procedure of Example 138, the reaction of Intermediate 11 (200 mg, 421 μmol) with 6-cyanonicotinic acid (69 mg, 463 μmol) was carried out. After purified by prep-HPLC (column: Waters xbridge 150*25 mm 10 μm; mobile phase: [water (NH₄HCO₃)-ACN]; B %: 35%-65%, 8 min), N-(1-(4-(2-(2-aminopyridin-3-yl)-5-phenyl-3H-imidazo[4,5-b]pyridin-3-yl)benzyl)piperidin-4-yl)-6-cyanonicotinamide (Example 167, 97.3 mg, yield: 37%) was obtained as a yellow solid. MS: m/z=606.2 [M+H]⁺. ¹H NMR (400 MHz, Dimethylsulfoxide-d₆) δ 9.11 (s, 1H), 8.71 (d, J=7.6 Hz, 1H), 8.40 (dd, J=8.0, 2.0 Hz, 1H), 8.27 (d, J=8.4 Hz, 1H), 8.17 (d, J=8.0 Hz, 1H), 8.08-7.96 (m, 4H), 7.51-7.37 (m, 7H), 7.16 (dd, J=7.6, 1.6 Hz, 1H), 7.04 (br s, 2H), 6.37 (dd, J=7.6, 4.8 Hz, 1H), 3.88-3.76 (m, 1H), 3.59 (s, 2H), 2.92-2.83 (m, 2H), 2.18-2.06 (m, 2H), 1.85 (d, J=10.8 Hz, 2H), 1.67-1.56 (m, 2H).

### Example 168: N-(4-(2-(2-Aminopyridin-3-yl)-6-phenyl-3H-imidazo[4,5-b]pyridin-3-yl)benzyl)-6-cyanopicolinamide

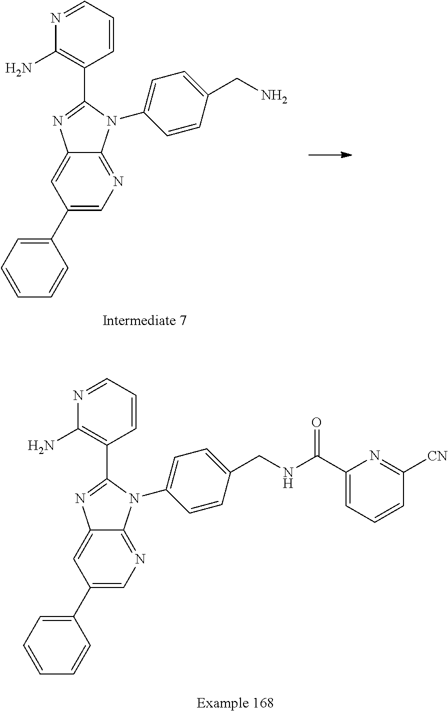 [SMILES: NCc1ccc(-n2c(-c3cccnc3N)nc3cc(-c4ccccc4)cnc32)cc1 ; N#Cc1cccc(C(=O)NCc2ccc(-n3c(-c4cccnc4N)nc4cc(-c5ccccc5)cnc43)cc2)n1]

Following the general procedure of Example 138, the reaction of Intermediate 7 (150 mg, 382 μmol) with 6-cyanopicolinic acid (62 mg, 420 μmol) was carried out. After purified by prep-HPLC (column: Waters xbridge 150*25 mm 10 μm; mobile phase: [water (NH₄HCO₃)-ACN]; B %: 39%-69%, 9 min), N-(4-(2-(2-Aminopyridin-3-yl)-6-phenyl-3H-imidazo[4,5-b]pyridin-3-yl)benzyl)-6-cyanopicolinamide (Example 168, 88.5 mg, yield: 44%) was obtained as a yellow solid. MS: m/z=523.2 [M+H]⁺. ¹H NMR (400 MHz, Dimethylsulfoxide-d₆) δ 9.61 (t, J=6.4 Hz, 1H), 8.60 (s, 1H), 8.46 (s, 1H), 8.36-8.23 (m, 3H), 8.04-7.98 (m, 1H), 7.77 (d, J=7.6 Hz, 2H), 7.55-7.40 (m, 7H), 7.29-7.23 (m, 1H), 7.01 (s, 2H), 6.45-6.41 (m, 1H), 4.59 (d, J=6.4 Hz, 2H).

### Example 169: N-(4-(2-(2-Aminopyridin-3-yl)-6-phenyl-3H-imidazo[4,5-b]pyridin-3-yl)benzyl)-2-cyanoisonicotinamide

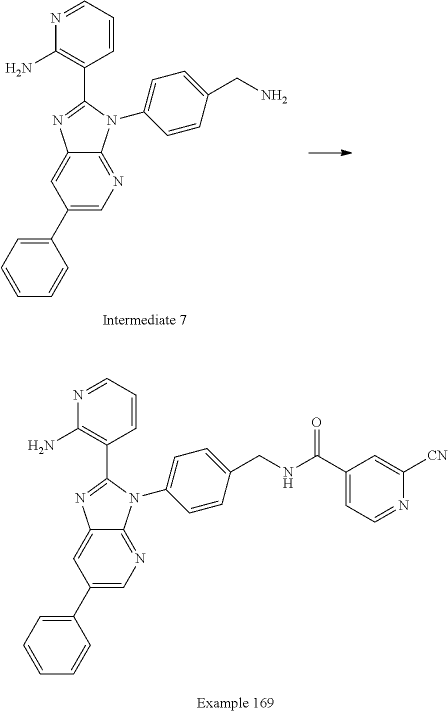 [SMILES: NCc1ccc(-n2c(-c3cccnc3N)nc3cc(-c4ccccc4)cnc32)cc1 ; N#Cc1cc(C(=O)NCc2ccc(-n3c(-c4cccnc4N)nc4cc(-c5ccccc5)cnc43)cc2)ccn1]

Following the general procedure of Example 138, the reaction of Intermediate 7 (150 mg, 382 μmol) with 2-cyanoisonicotinic acid (62 mg, 420 μmol) was carried out. After purified by prep-HPLC (column: Waters xbridge 150*25 mm 10 μm; mobile phase: [water (NH₄HCO₃)-ACN]; B %: 30%-60%, 8 min), N-(4-(2-(2-aminopyridin-3-yl)-6-phenyl-3H-imidazo[4,5-b]pyridin-3-yl)benzyl)-2-cyanoisonicotinamide (Example 169, 93.1 mg, yield: 46%) was obtained as a yellow solid. MS: m/z=523.10 [M+H]¹H NMR (400 MHz, Dimethylsulfoxide-d₆) δ 9.58 (t, J=5.6 Hz, 1H), 8.93 (d, J=5.2 Hz, 1H), 8.61 (s, 1H), 8.48-8.43 (m, 2H), 8.15 (d, J=5.2 Hz, 1H), 8.04-7.97 (m, 1H), 7.78 (d, J=7.2 Hz, 2H), 7.55-7.50 (m, 4H), 7.47-7.40 (m, 3H), 7.28 (dd, J=7.6, 2.0 Hz, 1H), 6.99 (br s, 2H), 6.43 (dd, J=7.6, 4.8 Hz, 1H), 4.62 (d, J=5.6 Hz, 2H).

### Example 170: N-(4-(2-(2-Aminopyridin-3-yl)-5-phenyl-3H-imidazo[4,5-b]pyridin-3-yl)benzyl)-4-cyanopicolinamide

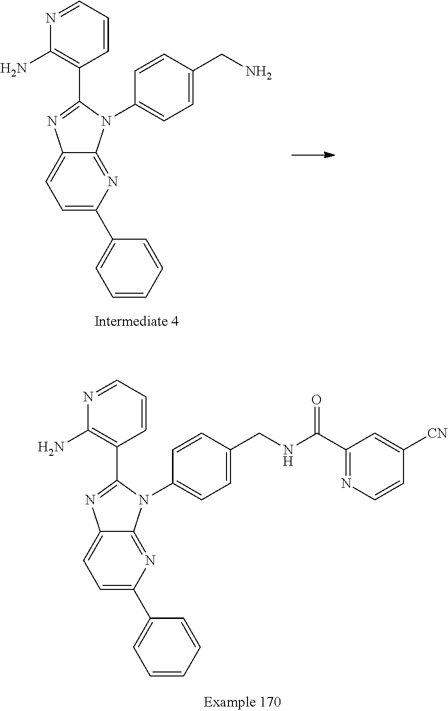 [SMILES: NCc1ccc(-n2c(-c3cccnc3N)nc3ccc(-c4ccccc4)nc32)cc1 ; N#Cc1ccnc(C(=O)NCc2ccc(-n3c(-c4cccnc4N)nc4ccc(-c5ccccc5)nc43)cc2)c1]

Following the general procedure of Example 138, the reaction of Intermediate 4 (200 mg, 510 μmol) with 4-cyanopyridine-2-carboxylic acid (83 mg, 560 μmol) in DCM was carried out. After purified by prep-HPLC (column: Waters xbridge 150×25 mm 10 μm; mobile phase: [water (NH₄HCO₃)-ACN]; B %: 37%-67%, 8 min), N-(4-(2-(2-aminopyridin-3-yl)-5-phenyl-3H-imidazo[4,5-b]pyridin-3-yl)benzyl)-4-cyanopicolinamide (Example 170, 31.7 mg, yield: 11%) was obtained as a yellow solid. MS: m/z=523.1 [M+H]⁺. ¹H NMR (400 MHz, Dimethylsulfoxide-d₆) δ 9.65 (t, J=6.4 Hz, 1H), 8.93 (d, J=4.8 Hz, 1H), 8.39 (s, 1H), 8.26 (d, J=8.0 Hz, 1H), 8.11 (dd, 1=5.2, 1.6 Hz, 1H), 8.04-7.96 (m, 4H), 7.50-7.43 (m, 6H), 7.41-7.37 (m, 1H), 7.21 (dd, J=7.6, 2.0 Hz, 1H), 6.93 (br s, 2H), 6.41 (dd, J=7.6, 4.8 Hz, 1H), 4.63 (d, J=6.4 Hz, 2H).

### Example 171: N-(4-(2-(2-Aminopyridin-3-yl)-5-phenyl-3H-imidazo[4,5-b]pyridin-3-yl)benzyl)-2-cyanopyrimidine-4-carboxamide

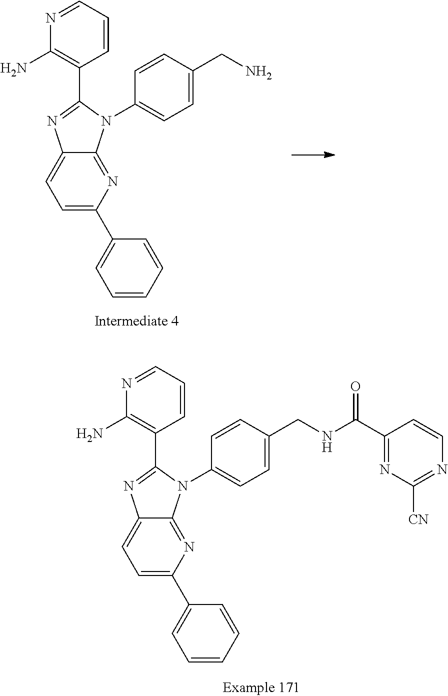 [SMILES: NCc1ccc(-n2c(-c3cccnc3N)nc3ccc(-c4ccccc4)nc32)cc1 ; N#Cc1nccc(C(=O)NCc2ccc(-n3c(-c4cccnc4N)nc4ccc(-c5ccccc5)nc43)cc2)n1]

Following the general procedure of Example 138, the reaction of Intermediate 4 (150 mg, 382 μmol) with 2-cyanopyrimidine-4-carboxylic acid (57 mg, 382 μmol) in DCM was carried out. After purified by prep-HPLC (column: Phenomenex 150×25 mm 10 μm; mobile phase: [water (NH₄HCO₃)-ACN]; B %: 42%-72%, 8 min), N-(4-(2-(2-aminopyridin-3-yl)-5-phenyl-3H-imidazo[4,5-b]pyridin-3-yl)benzyl)-2-cyanopyrimidine-4-carboxamide (Example 171, 19.1 mg, yield: 9.3%) was obtained as a yellow solid. MS: m/z=524.1 [M+H]⁺. ¹H NMR (400 MHz, Dimethylsulfoxide-d₆)) δ 9.88 (t, J=6.4 Hz, 1H), 9.25 (d, J=5.2 Hz, 1H), 8.32 (d, J=5.2 Hz, 1H), 8.26 (d, J=8.4 Hz, 1H), 8.03-7.96 (m, 4H), 7.52-7.44 (m, 6H), 7.41-7.36 (m, 1H), 7.21 (dd, J=7.6, 2.0 Hz, 1H), 6.91 (br s, 2H), 6.42 (dd, J=7.6, 4.8 Hz, 1H), 4.62 (d, J=6.4 Hz, 2H).

### Example 172: 4-(4-(4-(2-(2-Aminopyridin-3-yl)-5-phenyl-3H-imidazo[4,5-b]pyridin-3-yl)benzyl)piperazine-1-carbonyl)picolinonitrile

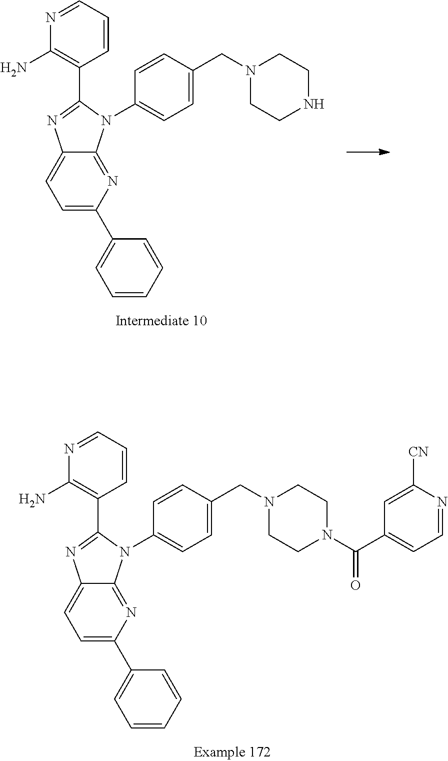 [SMILES: Nc1ncccc1-c1nc2ccc(-c3ccccc3)nc2n1-c1ccc(CN2CCNCC2)cc1 ; N#Cc1cc(C(=O)N2CCN(Cc3ccc(-n4c(-c5cccnc5N)nc5ccc(-c6ccccc6)nc54)cc3)CC2)ccn1]

Following the general procedure of Example 138, the reaction of Intermediate 10 (150 mg, 325 μmol) with 2-cyanoisonicotinic acid (58 mg, 390 μmol) in DCM was carried out. After purified by prep-HPLC (column: Waters xbridge 150*25 mm 10 μm; mobile phase: [water (NH₄HCO₃)-ACN]; B %: 40%-70%, 11 min), 4-(4-(4-(2-(2-aminopyridin-3-yl)-5-phenyl-3H-imidazo[4,5-b]pyridin-3-yl)benzyl)piperazine-1-carbonyl)picolinonitrile (Example 172, 50 mg, yield: 24%) was obtained as a white solid. MS: m/z=592.2 [M+H]⁺. ¹H NMR (400 MHz, Dimethylsulfoxide-d₆) δ 8.84 (d, J=4.8 Hz, 1H), 8.27 (d, J=8.4 Hz, 1H), 8.12 (s, 1H), 8.06-7.96 (m, 4H), 7.78-7.73 (m, 1H), 7.52-7.43 (m, 6H), 7.43-7.35 (m, 1H), 7.18-7.13 (m, 1H), 7.02 (br s, 2H), 6.43-6.36 (m, 1H), 3.71-3.60 (m, 4H), 3.32-3.27 (m, 2H), 2.44-2.38 (m, 4H).

### Example 173: N-(4-(2-(2-aminopyridin-3-yl)-3H-imidazo[4,5-b]pyridin-3-yl)benzyl)-2-cyano-5-fluorobenzo[d]thiazole-7-carboxamide

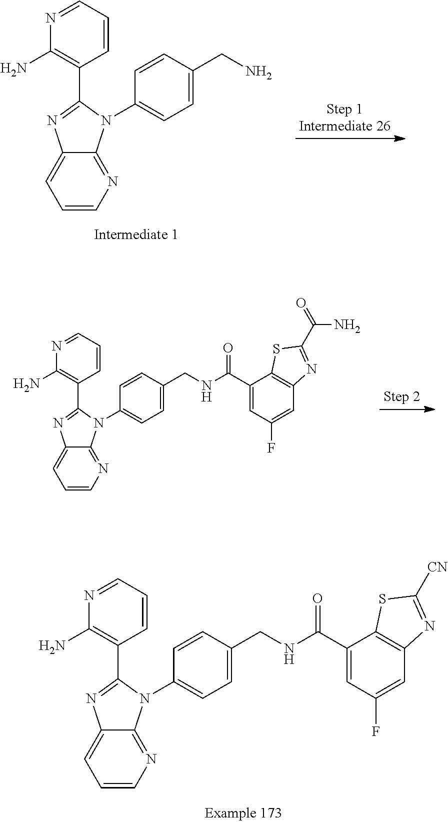 [SMILES: NCc1ccc(-n2c(-c3cccnc3N)nc3cccnc32)cc1 ; NC(=O)c1nc2cc(F)cc(C(=O)NCc3ccc(-n4c(-c5cccnc5N)nc5cccnc54)cc3)c2s1 ; N#Cc1nc2cc(F)cc(C(=O)NCc3ccc(-n4c(-c5cccnc5N)nc5cccnc54)cc3)c2s1]

### Step 1: N⁷-(4-(2-(2-Aminopyridin-3-yl)-3H-imidazo[4,5-b]pyridin-3-yl)benzyl)-5-fluorobenzo[d]thiazole-2,7-dicarboxamide

A mixture of Intermediate 1 (300 mg, 948 μmol), Intermediate 26 (227.8 mg, 948 μmol), HATU (540.9 mg, 1.42 mmol), DIEA (613 mg, 4.74 mmol) in DMF (2 mL) was stirred at 25° C. for 16 hr. This reaction was filtered and concentrated to give the residue. The residue was purified by prep-HPLC (column: Welch Xtimate C18 150×25 mm×5 μm; mobile phase: [water (NH₃H₂O)-ACN]; B %: 15%-45%, 8 min), N⁷-(4-(2-(2-aminopyridin-3-yl)-3H-imidazo[4,5-b]pyridin-3-yl)benzyl)-5-fluorobenzo[d]thiazole-2,7-dicarboxamide (39.5 mg, yield: 12%) was obtained as a light-yellow soild. MS: m/z=539.2 [M+H]⁺. ¹H NMR (400 MHz, Dimethylsulfoxide-d₆) δ 9.65 (t, J 5.6 Hz, 1H), 8.47 (s, 1H), 8.32-8.26 (m, 2H), 8.21-8.15 (m, 2H), 8.09 (s, 1H), 7.99 (dd, J=4.8, 1.6 Hz, 1H), 7.51 (d, J=8.0 Hz, 2H), 7.44-7.36 (m, 3H), 7.24 (dd, J=7.6, 1.6 Hz, 1H), 6.95 (br s, 2H), 6.41 (dd, J=7.6, 4.8 Hz, 1H), 4.67 (d, J 5.6 Hz, 2H). ¹⁹F NMR (400 MHz, Dimethylsulfoxide-d₆) δ −114.4.

### Step 2: N-(4-(2-(2-Aminopyridin-3-yl)-3H-imidazo[4,5-b]pyridin-3-yl)benzyl)-2-cyano-5-fluorobenzo[d]thiazole-7-carboxamide

To a solution of N⁷-(4-(2-(2-aminopyridin-3-yl)-3H-imidazo[4,5-b]pyridin-3-yl)benzyl)-5-fluorobenzo[d]thiazole-2,7-dicarboxamide (20 mg, 37.1 μmol) in pyridine (2 mL), then POCl₃ (142 mg, 928 μmol) was added dropwise over 5 min, the mixture was stirred at 20° C. for 2 hr. The reaction mixture was filtered to give filter liquor. The residue was purified by prep-HPLC (column: Welch Xtimate C18 150×25 mm×5 μm; mobile phase: [water (NH₄HCO₃)-ACN]; B %: 31%-61%, 2 min), N-(4-(2-(2-aminopyridin-3-yl)-3H-imidazo[4,5-b]pyridin-3-yl)benzyl)-2-cyano-5-fluorobenzo[d]thiazole-7-carboxamide (Example 173, 9.2 mg, yield: 47%) was obtained as a white lyophilized powder. MS: m/z=521.0 [M+H]f ¹H NMR (400 MHz, Dimethylsulfoxide-d₆) δ 9.83 (t, J=5.2 Hz, 1H), 8.51-8.41 (m, 2H), 8.30 (d, J=4.8 Hz, 1H), 8.19 (d, J=8.0 Hz, 1H), 8.02-7.96 (m, 1H), 7.51 (d, J=8.0 Hz, 2H), 7.43-7.35 (m, 3H), 7.24 (d, J=7.6 Hz, 1H), 6.93 (br s, 2H), 6.41 (dd, J=7.6, 4.8 Hz, 1H), 4.68 (d, J=5.2 Hz, 2H). ¹⁹F NMR (400 MHz, Dimethylsulfoxide-d₆) δ −112.9.

### Example 174: N-(4-(2-(2-aminopyridin-3-yl)-3H-imidazo[4,5-b]pyridin-3-yl)benzyl)-2-cyano-6-fluorobenzo[d]thiazole-4-carboxamide

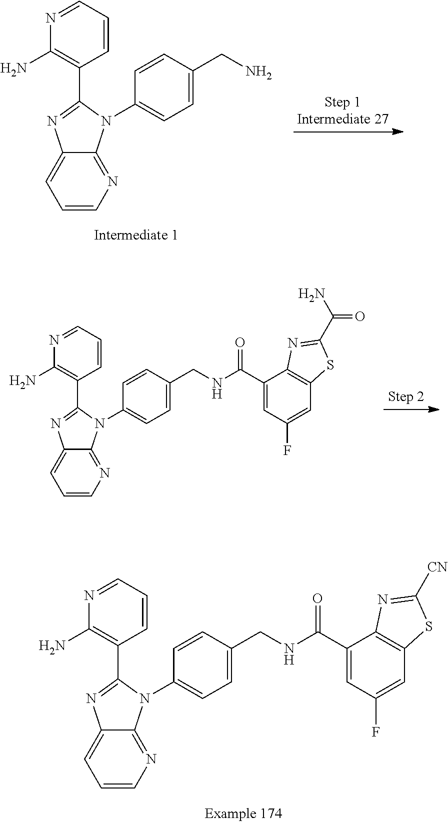 [SMILES: NCc1ccc(-n2c(-c3cccnc3N)nc3cccnc32)cc1 ; NC(=O)c1nc2c(C(=O)NCc3ccc(-n4c(-c5cccnc5N)nc5cccnc54)cc3)cc(F)cc2s1 ; N#Cc1nc2c(C(=O)NCc3ccc(-n4c(-c5cccnc5N)nc5cccnc54)cc3)cc(F)cc2s1]

### Step 1: N⁴-(4-(2-(2-Aminopyridin-3-yl)-3H-imidazo[4,5-b]pyridin-3-yl)benzyl)-6-fluorobenzo[d]thiazole-2,4-dicarboxamide

Following the general procedure of Example 173 step 1, the reaction of Intermediate 1 (50 mg, 158 μmol) with Intermediate 27 (38 mg, 158 μmol) was carried out. After purified by prep-HPLC (column: Welch Xtimate C18 150×25 mm×5 μm; mobile phase: [water (NH₄HCO₃)-ACN]; B %: 21%-51%, 10 min), N⁴-(4-(2-(2-aminopyridin-3-yl)-3H-imidazo[4,5-b]pyridin-3-yl)benzyl)-6-fluorobenzo[d]thiazole-2,4-dicarboxamide (30 mg, yield: 32%) was obtained as a light-yellow lyophilized powder. MS: m/z=539.1 [M+H]⁺. ¹H NMR (400 MHz, Dimethylsulfoxide-d₆) δ 10.05 (t, J=6.0 Hz, 1H), 9.13 (s, 1H), 8.42-8.35 (m, 1H), 8.32-8.27 (m, 2H), 8.19 (d, J 7.6 Hz, 1H), 8.11-8.05 (m, 1H), 8.00-7.96 (m, 1H), 7.48 (d, J=8.0 Hz, 2H), 7.41-7.34 (m, 3H), 7.24 (d, J=7.6 Hz, 1H), 6.95 (br s, 2H), 6.39 (dd, J=7.6, 4.8 Hz, 1H), 4.79 (br d, J=6.0 Hz, 2H). ¹⁹F NMR (400 MHz, Dimethylsulfoxide-d₆) δ −112.7.

### Step 2: N-(4-(2-(2-Aminopyridin-3-yl)-3H-imidazo[4,5-b]pyridin-3-yl)benzyl)-2-cyano-6-fluorobenzo[d]thiazole-4-carboxamide

Following the general procedure of Example 173 step 2, the reaction of N⁴-(4-(2-(2-aminopyridin-3-yl)-3H-imidazo[4,5-b]pyridin-3-yl)benzyl)-6-fluorobenzo[d]thiazole-2,4-dicarboxamide (30 mg, 56 μmol) with POCl₃ was carried out. After purified by prep-HPLC (column: Phenomenex C18 150×25 mm×10 μm; mobile phase: [water (NH₄HCO₃)-ACN]; B %: 30%-60%, 8 min), N-(4-(2-(2-aminopyridin-3-yl)-3H-imidazo[4,5-b]pyridin-3-yl)benzyl)-2-cyano-6-fluorobenzo[d]thiazole-4-carboxamide (Example 174, 4.8 mg, yield: 16%) was obtained as a light-yellow lyophilized powder. MS: m/z=521.1 [M+H]⁺. ¹H NMR (400 MHz, Methanol-d₄) δ 8.32 (dd, J=4.8, 1.6 Hz, 1H), 8.21-8.13 (m, 3H), 7.97 (dd, J=5.2, 2.0 Hz, 1H), 7.63 (d, J=8.4 Hz, 2H), 7.44-7.40 (m, 3H), 7.36-7.33 (m, 1H), 6.48 (dd, J=8.0, 5.2 Hz, 1H), 4.58 (s, 2H). ¹⁹F NMR (400 MHz, Methanol-d₄) δ −111.1.

### Example 175: 7-(4-(4-(2-(2-aminopyridin-3-yl)-3H-imidazo[4,5-b]pyridin-3-yl)benzyl)piperazine-1-carbonyl)-5-fluorobenzo[d]thiazole-2-carbonitrile

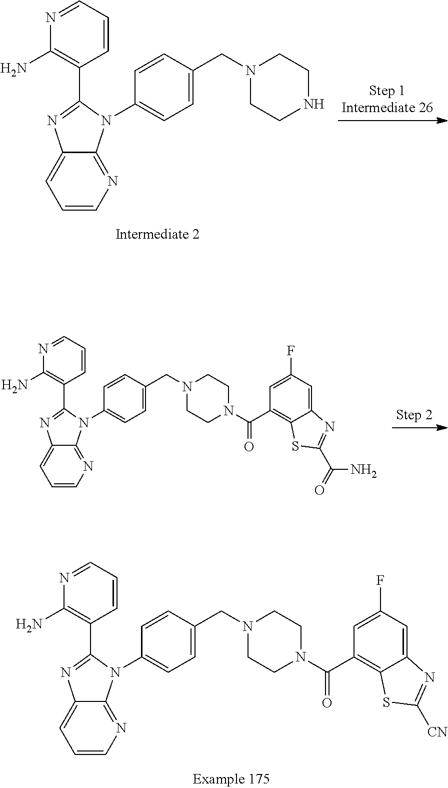 [SMILES: Nc1ncccc1-c1nc2cccnc2n1-c1ccc(CN2CCNCC2)cc1 ; NC(=O)c1nc2cc(F)cc(C(=O)N3CCN(Cc4ccc(-n5c(-c6cccnc6N)nc6cccnc65)cc4)CC3)c2s1 ; N#Cc1nc2cc(F)cc(C(=O)N3CCN(Cc4ccc(-n5c(-c6cccnc6N)nc6cccnc65)cc4)CC3)c2s1]

### Step 1: 7-(4-(4-(2-(2-Aminopyridin-3-yl)-3H-imidazo[4,5-b]pyridin-3-yl)benzyl)piperazine-1-carbonyl)-5-fluorobenzo[d]thiazole-2-carboxamide

Following the general procedure of Example 173 step 1, the reaction of Intermediate 2 (50 mg, 130 μmol) with Intermediate 26 (30 mg, 130 μmol) was carried out. After purified by prep-HPLC (column: Phenomenex C18 150×25 mm×10 μm; mobile phase: [water (NH₄HCO₃)-ACN]; B %: 20%-50%, 8 min), 7-(4-(4-(2-(2-aminopyridin-3-yl)-3H-imidazo[4,5-b]pyridin-3-yl)benzyl)piperazine-1-carbonyl)-5-fluorobenzo[d]thiazole-2-carboxamide (8.7 mg, yield: 11%) was obtained as an off-white lyophilized powder. MS: m/z=608.2 [M+H]⁺. ¹H NMR (400 MHz, Dimethylsulfoxide-d₆) δ 8.57 (s, 1H), 8.32 (dd, J=4.8, 1.2 Hz, 1H), 8.23-8.15 (m, 2H), 8.07 (dd, J=8.0, 2.4 Hz, 1H), 7.98 (dd, J=4.8, 2.0 Hz, 1H), 7.63 (dd, J=8.0, 2.4 Hz, 1H), 7.47 (d, J 8.4 Hz, 2H), 7.42-7.35 (m, 3H), 7.16 (dd, J 7.6, 2.0 Hz, 1H), 7.00 (br s, 2H), 6.37 (dd, J=7.6, 4.8 Hz, 1H), 3.67-3.56 (m, 4H), 3.31-3.22 (m, 2H), 2.49-2.39 (m, 4H). ¹⁹F NMR (400 MHz, Dimethylsulfoxide-d₆) δ −113.8.

### Step 2: 7-(4-(4-(2-(2-Aminopyridin-3-yl)-3H-imidazo[4,5-b]pyridin-3-yl)benzyl)piperazine-1-carbonyl)-5-fluorobenzo[d]thiazole-2-carbonitrile

Following the general procedure of Example 173 step 2, the reaction of 7-(4-(4-(2-(2-aminopyridin-3-yl)-3H-imidazo[4,5-b]pyridin-3-yl)benzyl)piperazine-1-carbonyl)-5-fluorobenzo[d]thiazole-2-carboxamide (30 mg, 49 μmol) with POCl₃ was carried out. After purified by prep-HPLC (column: Phenomenex C18 150×25 mm×10 μm; mobile phase: [water (NH₄HCO₃)-ACN]; B %: 30%-60%, 14 min), 7-(4-(4-(2-(2-aminopyridin-3-yl)-3H-imidazo[4,5-b]pyridin-3-yl)benzyl)piperazine-1-carbonyl)-5-fluorobenzo[d]thiazole-2-carbonitrile (Example 175, 2.3 mg, yield: 7.4%) was obtained as light-yellow lyophilized powder. MS: m/z=590.3 [M+H]⁺. ¹H NMR (400 MHz, Methanol-d₄) δ 8.34 (dd, J=4.8, 1.2 Hz, 1H), 8.20 (dd, J=8.0, 5.2 Hz, 1H), 8.14-8.09 (m, 1H), 7.97 (dd, J=4.8, 1.6 Hz, 1H), 7.72 (dd, J=8.8, 2.4 Hz, 1H), 7.55 (d, J=8.4 Hz, 2H), 7.45-7.41 (m, 1H), 7.40-7.38 (m, 2H), 7.32 (dd, J=7.6, 2.0 Hz, 1H), 6.46 (dd, J=7.6, 4.8 Hz, 1H), 3.81-3.70 (m, 4H), 3.68 (s, 2H), 2.60-2.52 (m, 4H). ¹⁹F NMR (400 MHz, Methanol-d₄) δ −113.8.

### Example 176: N-(4-(2-(2-Aminopyridin-3-yl)-5-phenyl-3H-imidazo[4,5-b]pyridin-3-yl)benzyl)-2-cyano-5-fluorobenzo[d]thiazole-7-carboxamide

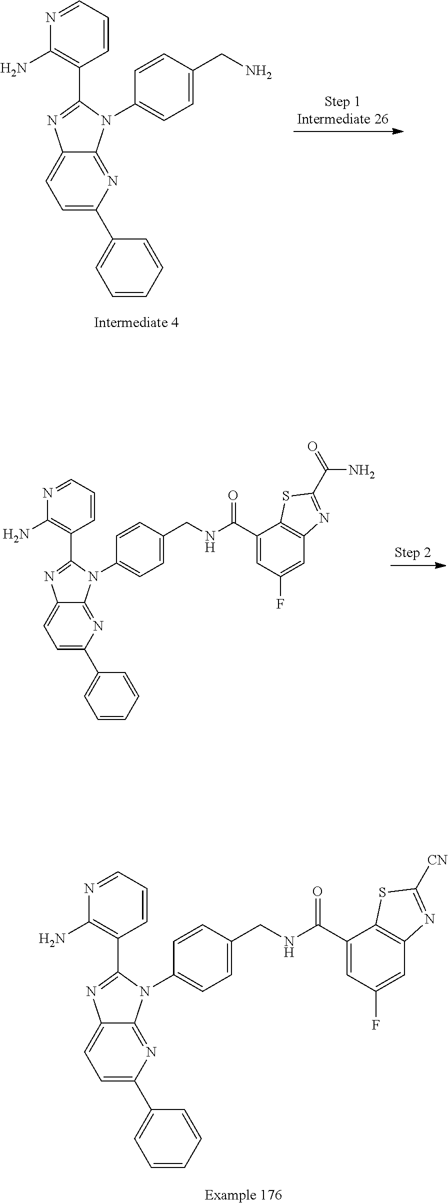 [SMILES: NCc1ccc(-n2c(-c3cccnc3N)nc3ccc(-c4ccccc4)nc32)cc1 ; NC(=O)c1nc2cc(F)cc(C(=O)NCc3ccc(-n4c(-c5cccnc5N)nc5ccc(-c6ccccc6)nc54)cc3)c2s1 ; N#Cc1nc2cc(F)cc(C(=O)NCc3ccc(-n4c(-c5cccnc5N)nc5ccc(-c6ccccc6)nc54)cc3)c2s1]

### Step 1: N⁷-(4-(2-(2-Aminopyridin-3-yl)-5-phenyl-3H-imidazo[4,5-b]pyridin-3-yl)benzyl)-5-fluorobenzo[d]thiazole-2,7-dicarboxamide

Following the general procedure of Example 173 step 1, the reaction of Intermediate 4 (50 mg, 127 μmol) with Intermediate 26 (30.6 mg, 127 μmol) was carried out. After purified by prep-HPLC (column: Welch Xtimate C18 150×25 mm×5 μm; mobile phase: [water (NH₃H₂O)-ACN]; B %: 35%-65%, 8 min), N⁷-(4-(2-(2-aminopyridin-3-yl)-5-phenyl-3H-imidazo[4,5-b]pyridin-3-yl)benzyl)-5-fluorobenzo[d]thiazole-2,7-dicarboxamide (35 mg, yield: 41%) was obtained as a yellow lyophilized powder. MS: m/z=615.3 [M+H]⁺. ¹H NMR (400 MHz, Dimethylsulfoxide-d₆) δ 10.10-10.03 (m, 1H), 9.15 (s, 1H), 8.39 (dd, J=8.0, 2.8 Hz, 1H), 8.32-8.28 (m, 1H), 8.26 (d, J=8.4 Hz, 1H), 8.09 (dd, J=9.6, 2.8 Hz, 1H), 8.03-7.96 (m, 4H), 7.52-7.44 (m, 6H), 7.41-7.35 (m, 1H), 7.23 (dd, J=7.6, 1.6 Hz, 1H), 6.91 (br s, 2H), 6.41 (dd, J=7.6, 4.8 Hz, 1H), 4.83 (d, J=6.8 Hz, 2H). ¹⁹F NMR (400 MHz, Dimethylsulfoxide-d₆) δ −112.7.

### Step 2; N-(4-(2-(2-Aminopyridin-3-yl)-5-phenyl-3H-imidazo[4,5-b]pyridin-3-yl)benzyl)-2-cyano-5-fluorobenzo[d]thiazole-7-carboxamide

Following the general procedure of Example 173 step 2, the reaction of N⁷-(4-(2-(2-aminopyridin-3-yl)-5-phenyl-3H-imidazo[4,5-b]pyridin-3-yl)benzyl)-5-fluorobenzo[d]thiazole-2,7-dicarboxamide (30 mg, 48 μmol) was carried out. After purified by prep-HPLC (column: Phenomenex C18 150×25 mm×10 μm; mobile phase: [water (NH₄HCO₃)-ACN]; B %: 36%-66%, 8 min), N-(4-(2-(2-Aminopyridin-3-yl)-5-phenyl-3H-imidazo[4,5-b]pyridin-3-yl)benzyl)-2-cyano-5-fluorobenzo[d]thiazole-7-carboxamide (Example 176, 4.5 mg, yield: 15%) was obtained as a light-yellow lyophilized powder. MS: m/z=597.1 [M+H]⁺. ¹H NMR (400 MHz, Methanol-d₄) δ 8.21-8.15 (m, 3H), 8.03-8.00 (m, 2H), 7.97 (dd, J=4.8, 1.6 Hz, 1H), 7.93 (d, J=8.4 Hz, 1H), 7.59 (d, J=8.4 Hz, 2H), 7.47 (d, J=8.4 Hz, 2H), 7.43-7.37 (m, 3H), 7.36-7.32 (m, 2H), 6.48 (dd, J=7.6, 5.2 Hz, 1H), 4.77 (s, 2H). ¹⁹F NMR (400 MHz, Methanol-d₄) δ −114.4.

### Example 177: N-(4-(2-(2-Aminopyridin-3-yl)-5-phenyl-3H-imidazo[4,5-b]pyridin-3-yl)benzyl)-2-cyano-6-fluorobenzo[d]thiazole-4-carboxamide

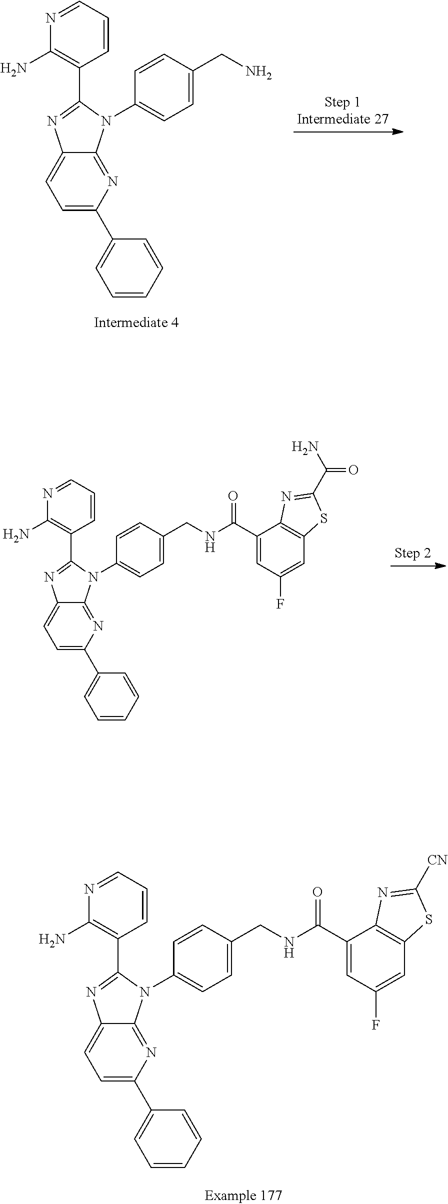 [SMILES: NCc1ccc(-n2c(-c3cccnc3N)nc3ccc(-c4ccccc4)nc32)cc1 ; NC(=O)c1nc2c(C(=O)NCc3ccc(-n4c(-c5cccnc5N)nc5ccc(-c6ccccc6)nc54)cc3)cc(F)cc2s1 ; N#Cc1nc2c(C(=O)NCc3ccc(-n4c(-c5cccnc5N)nc5ccc(-c6ccccc6)nc54)cc3)cc(F)cc2s1]

### Step 1: N-(4-(2-(2-Aminopyridin-3-yl)-5-phenyl-3H-imidazo[4,5-b]pyridin-3-yl)benzyl)-6-fluorobenzo[d]thiazole-2,4-dicarboxamide

Following the general procedure of Example 173 step 1, the reaction of Intermediate 4 (50 mg, 127 μmol) with Intermediate 27 (30.6 mg, 127 μmol) was carried out. After purified by prep-HPLC (column: Welch Xtimate C18 150×25 mm×5 μm; mobile phase: [water (NH₃H₂O)-ACN]; B %: 36%-66%, 8 min), N⁴-(4-(2-(2-aminopyridin-3-yl)-5-phenyl-3H-imidazo[4,5-b]pyridin-3-yl)benzyl)-6-fluorobenzo[d]thiazole-2,4-dicarboxamide (40 mg, yield: 46%) was obtained as a yellow lyophilized powder. MS: m/z=615.4 [M+H]⁺. ¹H NMR (400 MHz, Dimethylsulfoxide-d₆) δ 10.06 (t, J 6.0, 1H), 9.14 (s, 1H), 8.39 (dd, J=7.8, 2.8 Hz, 1H), 8.30 (s, 1H), 8.26 (d, J=8.4 Hz, 1H), 8.09 (dd, J=10.0, 2.8 Hz, 1H), 8.03-7.96 (m, 4H), 7.52-7.43 (m, 6H), 7.41-7.35 (m, 1H), 7.23 (dd, J=7.6, 1.6 Hz, 1H), 6.91 (br s, 2H), 6.41 (dd, J=7.6, 4.8 Hz, 1H), 4.83 (d, J=6.0 Hz, 2H). ¹⁹F NMR (400 MHz, Dimethylsulfoxide-d₆) δ −112.7.

### Step 2: N-(4-(2-(2-Aminopyridin-3-yl)-5-phenyl-3H-imidazo[4,5-b]pyridin-3-yl)benzyl)-2-cyano-6-fluorobenzo[d]thiazole-4-carboxamide

Following the general procedure of Example 173 step 2, the reaction of N⁴-(4-(2-(2-aminopyridin-3-yl)-5-phenyl-3H-imidazo[4,5-b]pyridin-3-yl)benzyl)-6-fluorobenzo[d]thiazole-2,4-dicarboxamide (40 mg, 65 μmol) with POCl₃ was carried out. After purified by prep-HPLC (column: Phenomenex C18 150×25 mm×10 μm; mobile phase: [water (NH₄HCO₃)-ACN]; B %: 35%-60%, 8 min), N-(4-(2-(2-aminopyridin-3-yl)-5-phenyl-3H-imidazo[4,5-b]pyridin-3-yl)benzyl)-2-cyano-6-fluorobenzo[d]thiazole-4-carboxamide (Example 177, 11.4 mg, yield: 28%) was obtained as a yellow lyophilized powder. MS: m/z=597.3 [M+H]⁺. ¹H NMR (400 MHz, Dimethylsulfoxide-d₆) 9.60-9.55 (m, 1H), 8.46-8.42 (m, 1H), 8.26 (d, J 8.4 Hz, 1H), 8.07-7.94 (m, 6H), 7.59-7.57 (m, 2H), 7.50-7.43 (m, 4H), 7.41-7.36 (m, 1H), 7.24-7.21 (m, 1H), 6.93 (br s, 2H), 6.46-6.40 (m, 1H), 4.77 (d, J=5.6 Hz, 2H). ¹⁹F NMR (400 MHz, Dimethylsulfoxide-d₆) δ −109.8.

### Example 178: N-(4-(5-(3-Acetamidophenyl)-2-(2-aminopyridin-3-yl)-3H-imidazo[4,5-b]pyridin-3-yl)benzyl)-2-cyano-5-fluorobenzo[d]thiazole-7-carboxamide

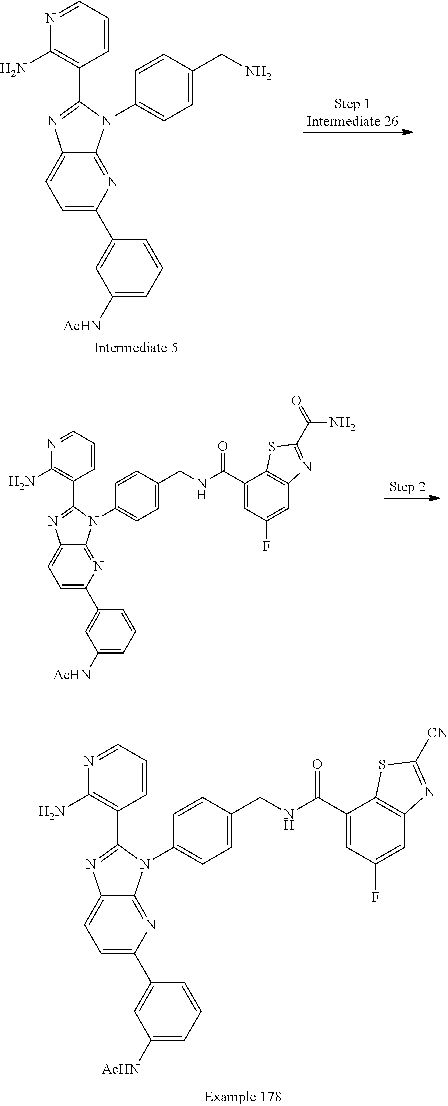 [SMILES: CC(=O)Nc1cccc(-c2ccc3nc(-c4cccnc4N)n(-c4ccc(CN)cc4)c3n2)c1 ; CC(=O)Nc1cccc(-c2ccc3nc(-c4cccnc4N)n(-c4ccc(CNC(=O)c5cc(F)cc6nc(C(N)=O)sc56)cc4)c3n2)c1 ; CC(=O)Nc1cccc(-c2ccc3nc(-c4cccnc4N)n(-c4ccc(CNC(=O)c5cc(F)cc6nc(C#N)sc56)cc4)c3n2)c1]

### Step 1 N⁷-(4-(5-(3-acetamidophenyl)-2-(2-aminopyridin-3-yl)-3H-imidazo[4,5-b]pyridin-3-yl)benzyl)-5-fluorobenzo[d]thiazole-2,7-dicarboxamide

Following the general procedure of Example 173 step 1, the reaction of Intermediate 5 (200 mg, 445 μmol) with Intermediate 26 (106.9 mg, 445 μmol) was carried out. After purified by prep-HPLC (column: Phenomenex C18 150×25 mm×10 μm; mobile phase: [water (NH₄HCO₃)-ACN]; B %: 26%-56%, 8 min), N⁷-(4-(5-(3-acetamidophenyl)-2-(2-aminopyridin-3-yl)-3H-imidazo[4,5-b]pyridin-3-yl)benzyl)-5-fluorobenzo[d]thiazole-2,7-dicarboxamide (30 mg, yield: 7.9%) was obtained as an off-white lyophilized powder. MS: m/z=672.4

### Step 2: N-(4-(5-(3-Acetamidophenyl)-2-(2-aminopyridin-3-yl)-3H-imidazo[4,5-b]pyridin-3-yl)benzyl)-2-cyano-5-fluorobenzo[d]thiazole-7-carboxamide

Following the general procedure of Example 173 step 2, the reaction of N⁷-(4-(5-(3-acetamidophenyl)-2-(2-aminopyridin-3-yl)-3H-imidazo[4,5-b]pyridin-3-yl)benzyl)-5-fluorobenzo[d]thiazole-2,7-dicarboxamide (30 mg, 45 μmol) with POCl₃ was carried out. After purified by prep-HPLC (column: Phenomenex C18 150×25 mm×10 μm; mobile phase: [water(NH₄HCO₃)-ACN]; B %: 33%-63%, 14 min), N-(4-(5-(3-acetamidophenyl)-2-(2-aminopyridin-3-yl)-3H-imidazo[4,5-b]pyridin-3-yl)benzyl)-2-cyano-5-fluorobenzo[d]thiazole-7-carboxamide (Example 178, 3.4 mg, yield: 11%) was obtained as an off-white lyophilized powder. MS: m/z=654.2 [M+H]⁺. ¹H NMR (400 MHz, Methanol-d₄) δ 8.23-8.16 (m, 4H), 7.98 (dd, J=5.2, 1.6 Hz, 1H), 7.91 (d, J=8.4 Hz, 1H), 7.76-7.74 (m, 1H), 7.62-7.57 (m, 3H), 7.48 (d, J=8.4 Hz, 2H), 7.38-7.33 (m, 2H), 6.48 (dd, J=7.6, 4.8 Hz, 1H), 4.77 (s, 2H), 2.11 (s, 3H).

### Example 179: N-(4-(5-(3-acetamidophenyl)-2-(2-aminopyridin-3-yl)-3H-imidazo[4,5-b]pyridin-3-yl)benzyl)-2-cyano-6-fluorobenzo[d]thiazole-4-carboxamide

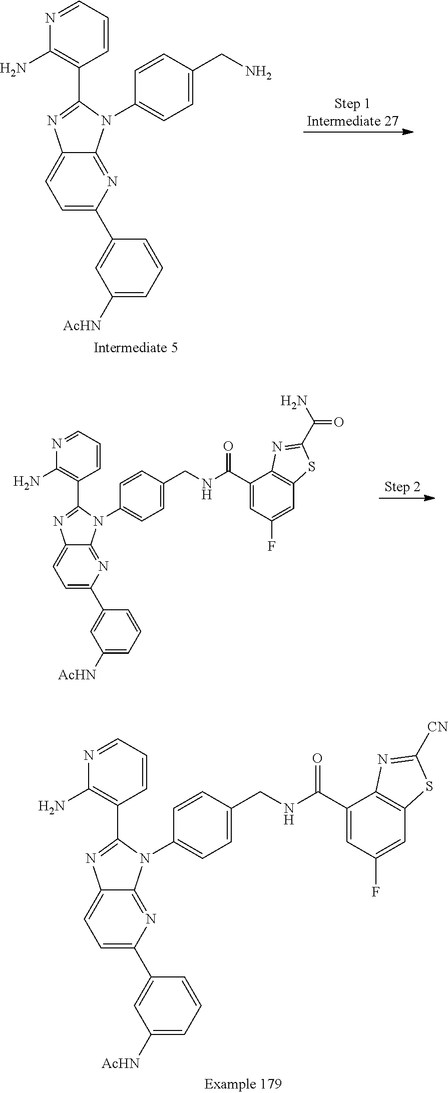 [SMILES: CC(=O)Nc1cccc(-c2ccc3nc(-c4cccnc4N)n(-c4ccc(CN)cc4)c3n2)c1 ; CC(=O)Nc1cccc(-c2ccc3nc(-c4cccnc4N)n(-c4ccc(CNC(=O)c5cc(F)cc6sc(C(N)=O)nc56)cc4)c3n2)c1 ; CC(=O)Nc1cccc(-c2ccc3nc(-c4cccnc4N)n(-c4ccc(CNC(=O)c5cc(F)cc6sc(C#N)nc56)cc4)c3n2)c1]

### Step 1: N⁴-(4-(5-(3-Acetamidophenyl)-2-(2-aminopyridin-3-yl)-3H-imidazo[4,5-b]pyridin-3-yl)benzyl)-6-fluorobenzo[d]thiazole-2,4-dicarboxamide

Following the general procedure of Example 173 step 1, the reaction of Intermediate 5 (50 mg, 111 μmol) with Intermediate 27 (26.7 mg, 111 μmol) was carried out. After purified by prep-HPLC (column: Welch Xtimate C18 150×25 mm×5 μm; mobile phase: [water (ammonia hydroxide v/v)-ACN]; B %: 25%-55%, 2 min), N⁴-(4-(5-(3-acetamidophenyl)-2-(2-aminopyridin-3-yl)-3H-imidazo[4,5-b]pyridin-3-yl)benzyl)-6-fluorobenzo[d]thiazole-2,4-dicarboxamide (30 mg, yield: 40%) was obtained as a yellow lyophilized powder. MS: m/z=672.5 [M+H]⁺. ¹H NMR (400 MHz, Dimethylsulfoxide-d₆) δ 10.09-10.04 (m, 2H), 9.15 (s, 1H), 8.39 (dd, J=7.6, 2.8 Hz, 1H), 8.31 (s, 1H), 8.27 (d, J=8.4 Hz, 1H), 8.12-8.06 (m, 2H), 7.98 (dd, J=4.8, 2.0 Hz, 1H), 7.83 (d, J=8.4 Hz, 1H), 7.71-7.67 (m, 1H), 7.65-7.62 (m, 1H), 7.51-7.44 (m, 4H), 7.39-7.34 (m, 1H), 7.22 (dd, J=7.6, 1.6 Hz, 1H), 6.85 (br s, 2H), 6.40 (dd, J=7.6, 4.8 Hz, 1H), 4.82 (d, J=6.0 Hz, 2H), 2.02 (s, 3H). ¹⁹F NMR (400 MHz, Dimethylsulfoxide-d₆) δ −112.7.

### Step 2: N-(4-(5-(3-Acetamidophenyl)-2-(2-aminopyridin-3-yl)-3H-imidazo[4,5-b]pyridin-3-yl)benzyl)-2-cyano-6-fluorobenzo[d]thiazole-4-carboxamide

Following the general procedure of Example 173 step 2, the reaction of M-(4-(5-(3-acetamidophenyl)-2-(2-aminopyridin-3-yl)-3H-imidazo[4,5-b]pyridin-3-yl)benzyl)-6-fluorobenzo[d]thiazole-2,4-dicarboxamide (30 mg, 45 μmol) with POCl₃ was carried out. After purified by prep-HPLC (column: Waters Xbridge BEH C18 150×25 mm×5 μm; mobile phase: [water-ACN]; B %: 43%-73%, 10 min), N-(4-(5-(3-acetamidophenyl)-2-(2-aminopyridin-3-yl)-3H-imidazo[4,5-b]pyridin-3-yl)benzyl)-2-cyano-6-fluorobenzo[d]thiazole-4-carboxamide (Example 179, 6.1 mg, yield: 20%) was obtained as a light-yellow lyophilized powder. MS: m/z=654.4 [M+H]⁺. ¹H NMR (400 MHz, Methanol-d₄) δ 8.21-8.13 (m, 4H), 7.98-7.90 (m, 1H), 7.91 (d, J=8.0 Hz, 1H), 7.77-7.74 (m, 1H), 7.67-7.61 (m, 3H), 7.48 (d, J=8.4 Hz, 2H), 7.39-7.32 (m, 2H), 6.51 (dd, J=7.6, 4.8 Hz, 1H), 4.79 (s, 2H), 2.12 (s, 3H). ¹⁹F NMR (400 MHz, Methanol-d₄) δ −111.1.

### Example 180: 7-(4-(4-(2-(2-Aminopyridin-3-yl)-5-phenyl-3H-imidazo[4,5-b]pyridin-3-yl)benzyl)piperazine-1-carbonyl)-5-fluorobenzo[d]thiazole-2-carbonitrile

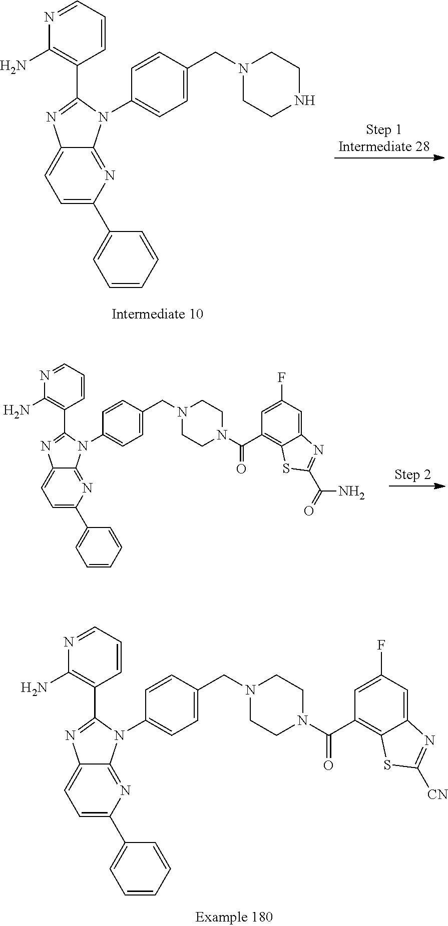 [SMILES: Nc1ncccc1-c1nc2ccc(-c3ccccc3)nc2n1-c1ccc(CN2CCNCC2)cc1 ; NC(=O)c1nc2cc(F)cc(C(=O)N3CCN(Cc4ccc(-n5c(-c6cccnc6N)nc6ccc(-c7ccccc7)nc65)cc4)CC3)c2s1 ; N#Cc1nc2cc(F)cc(C(=O)N3CCN(Cc4ccc(-n5c(-c6cccnc6N)nc6ccc(-c7ccccc7)nc65)cc4)CC3)c2s1]

### Step 1: 7-(4-(4-(2-(2-Aminopyridin-3-yl)-5-phenyl-3H-imidazo[4,5-b]pyridin-3-yl)benzyl)piperazine-1-carbonyl)-5-fluorobenzo[d]thiazole-2-carboxamide

Following the general procedure of Example 173 step 1, the reaction of Intermediate 10 (200 mg, 433 μmol) with Intermediate 26 (104 mg, 433 μmol) was carried out. After purified by prep-HPLC (column: Waters xbridge 150×25 mm 10 μm; mobile phase: [water (NH₄HCO₃)-ACN]; B %: 35%-65%, 11 min), 7-(4-(4-(2-(2-aminopyridin-3-yl)-5-phenyl-3H-imidazo[4,5-b]pyridin-3-yl)benzyl)piperazine-1-carbonyl)-5-fluorobenzo[d]thiazole-2-carboxamide (40 mg, yield: 13%) was obtained as an off-white solid. MS: m/z=684.5 [M+H]⁺. ¹H NMR (400 MHz, Dimethylsulfoxide-d₆) δ 8.57 (s, 1H), 8.27 (d, J 8.4 Hz, 1H), 8.17 (s, 1H), 8.07 (dd, J=9.2, 2.4 Hz, 1H), 8.04-8.00 (m, 2H), 8.00-7.97 (m, 2H), 7.63 (dd, J=9.2, 2.4 Hz, 1H), 7.53-7.49 (m, 2H), 7.48-7.43 (m, 4H), 7.42-7.36 (m, 1H), 7.15 (dd, J 7.6, 1.6 Hz, 1H), 7.01 (br s, 2H), 6.38 (dd, J=7.6, 4.8 Hz, 1H), 3.67-3.58 (m, 4H), 3.32-3.27 (m, 2H), 2.63-2.55 (m, 2H), 2.47-2.39 (m, 2H). ¹⁹F NMR (400 MHz, Dimethylsulfoxide-d₆) δ −113.8.

### Step 2: 7-(4-(4-(2-(2-Aminopyridin-3-yl)-5-phenyl-3H-imidazo[4,5-b]pyridin-3-yl)benzyl)piperazine-1-carbonyl)-5-fluorobenzo[d]thiazole-2-carbonitrile

Following the general procedure of Example 173 step 2, the reaction of 7-(4-(4-(2-(2-aminopyridin-3-yl)-5-phenyl-3H-imidazo[4,5-b]pyridin-3-yl)benzyl)piperazine-1-carbonyl)-5-fluorobenzo[d]thiazole-2-carboxamide (30 mg, 44 μmol) with POCl₃ was carried out. After purified by prep-HPLC (column: Phenomenex C18 150×25 mm×10 μm; mobile phase: [water (NH₄HCO₃)-ACN]; B %: 48%-78%, 8 min), 7-(4-(4-(2-(2-aminopyridin-3-yl)-5-phenyl-3H-imidazo[4,5-b]pyridin-3-yl)benzyl)piperazine-1-carbonyl)-5-fluorobenzo[d]thiazole-2-carbonitrile (Example 180, 3.8 mg, yield: 13%) was obtained as a light-yellow lyophilized powder. MS: m/z=666.4 [M+H]⁺. ¹H NMR (400 MHz, Dimethylsulfoxide-d₆) δ 8.34 (dd, J=8.8, 2.4 Hz, 1H), 8.27 (d, J=8.4 Hz, 1H), 8.04-7.96 (m, 4H), 7.85 (dd, J=9.2, 2.0 Hz, 1H), 7.53-7.49 (m, 2H), 7.49-7.43 (m, 4H), 7.42-7.36 (m, 1H), 7.16 (dd, J=7.6, 1.8 Hz, 1H), 7.01 (br s, 2H), 6.38 (dd, J=7.6, 4.8 Hz, 1H), 3.75-3.59 (m, 6H), 3.44-3.40 (m, 4H). ¹⁹F NMR (400 MHz, Dimethylsulfoxide-d₆) δ −112.4.

### Example 181: 4-(4-(4-(2-(2-Aminopyridin-3-yl)-5-phenyl-3H-imidazo[4,5-b]pyridin-3-yl)benzyl)piperazine-1-carbonyl)-6-fluorobenzo[d]thiazole-2-carbonitrile

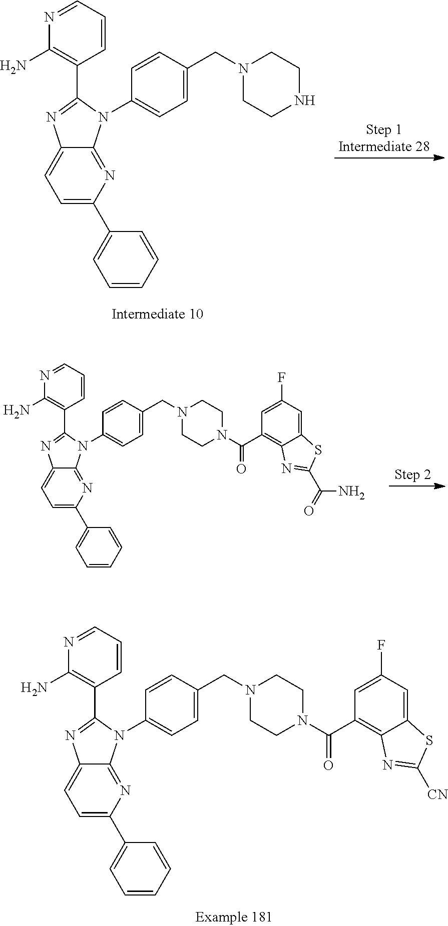 [SMILES: Nc1ncccc1-c1nc2ccc(-c3ccccc3)nc2n1-c1ccc(CN2CCNCC2)cc1 ; NC(=O)c1nc2c(C(=O)N3CCN(Cc4ccc(-n5c(-c6cccnc6N)nc6ccc(-c7ccccc7)nc65)cc4)CC3)cc(F)cc2s1 ; N#Cc1nc2c(C(=O)N3CCN(Cc4ccc(-n5c(-c6cccnc6N)nc6ccc(-c7ccccc7)nc65)cc4)CC3)cc(F)cc2s1]

### Step 1: 4-(4-(4-(2-(2-aminopyridin-3-yl)-5-phenyl-3H-imidazo[4,5-b]pyridin-3-yl)benzyl)piperazine-1-carbonyl)-6-fluorobenzo[d]thiazole-2-carboxamide

Following the general procedure of Example 173 step 1, the reaction of Intermediate 10 (50 mg, 108 μmol) with Intermediate 27 (26 mg, 108 μmol) was carried out. After purified by prep-HPLC (column: Welch Xtimate C18 150×25 mm×5 μm; mobile phase: [water (NH₃H₂O)-ACN]; B %; 35%-65%, 8 min), 4-(4-(4-(2-(2-aminopyridin-3-yl)-5-phenyl-3H-imidazo[4,5-b]pyridin-3-yl)benzyl)piperazine-1-carbonyl)-6-fluorobenzo[d]thiazole-2-carboxamide (15.3 mg yield: 20%) was obtained as light yellow lyophilized powder. MS: m/z=684.4 [M+H]⁺. ¹H NMR (400 MHz, Dimethylsulfoxide-d₆) δ 8.28-8.24 (m, 2H), 8.22-8.16 (m, 2H), 8.03-7.96 (m, 4H), 7.53-7.36 (m, 8H), 7.15 (d, J=7.2 Hz, 1H), 7.01 (br s, 2H), 6.37 (dd, J=7.6, 4.8 Hz, 1H), 4.00-3.86 (m, 1H), 3.80-3.47 (m, 4H), 3.24-3.11 (m, 3H), 2.81-2.70 (m, 1H), 2.28-2.16 (m, 1H). ¹⁹F NMR (400 MHz, Dimethylsulfoxide-d₆) δ −112.8.

### Step 2: 4-(4-(4-(2-(2-Aminopyridin-3-yl)-5-phenyl-3H-imidazo[4,5-b]pyridin-3-yl)benzyl)piperazine-1-carbonyl)-6-fluorobenzo[d]thiazole-2-carbonitrile

Following the general procedure of Example 173 step 2, the reaction of 4-(4-(4-(2-(2-aminopyridin-3-yl)-5-phenyl-3H-imidazo[4,5-b]pyridin-3-yl)benzyl)piperazine-1-carbonyl)-6-fluorobenzo[d]thiazole-2-carboxamide (30 mg, 44 μmol) with POCl₃ was carried out. After purified by prep-HPLC (column: Phenomenex C18 150×25 mm×10 μm; mobile phase: [water (NH₄HCO₃)-ACN]; B %: 50%-80%, 8 min), 4-(4-(4-(2-(2-aminopyridin-3-yl)-5-phenyl-3H-imidazo[4,5-b]pyridin-3-yl)benzyl)piperazine-1-carbonyl)-6-fluorobenzo[d]thiazole-2-carbonitrile (Example 181, 4.8 mg, yield: 16%) was obtained as light-yellow lyophilized powder. MS: m/z=666.1 [M+H]⁺. ¹H NMR (400 MHz, Methanol-d₄) δ 8.19 (d, J 8.4 Hz, 1H), 8.06 (dd, J=8.0, 2.4 Hz, 1H), 8.04-8.00 (m, 2H), 7.97-7.91 (m, 2H), 7.58-7.52 (m, 3H), 7.47-7.33 (m, 5H), 7.32 (dd, J=7.6, 2.0 Hz, 1H), 6.46 (dd, J=7.6, 4.8 Hz, 1H), 4.60 (s, 2H), 3.99-3.84 (m, 2H), 3.70 (s, 2H), 2.73-2.65 (m, 2H), 2.53-2.43 (m, 2H). ¹⁹F NMR (400 MHz, Methanol-d₄) δ −111.0.

### Example 182: N-(1-(4-(2-(2-aminopyridin-3-yl)-5-phenyl-3H-imidazo[4,5-b]pyridin-3-yl)benzyl)piperidin-4-yl)-2-cyano-5-fluorobenzo[d]thiazole-7-carboxamide

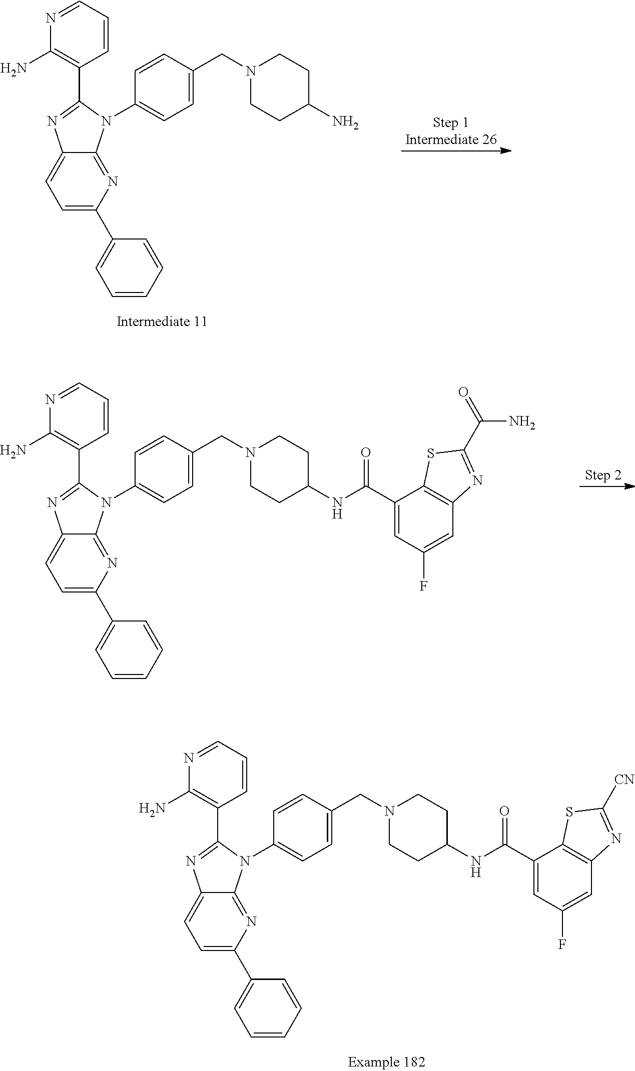 [SMILES: Nc1ncccc1-c1nc2ccc(-c3ccccc3)nc2n1-c1ccc(CN2CCC(N)CC2)cc1 ; NC(=O)c1nc2cc(F)cc(C(=O)NC3CCN(Cc4ccc(-n5c(-c6cccnc6N)nc6ccc(-c7ccccc7)nc65)cc4)CC3)c2s1 ; N#Cc1nc2cc(F)cc(C(=O)NC3CCN(Cc4ccc(-n5c(-c6cccnc6N)nc6ccc(-c7ccccc7)nc65)cc4)CC3)c2s1]

### Step 1: N⁷-(1-(4-(2-(2-aminopyridin-3-yl)-5-phenyl-3H-imidazo[4,5-b]pyridin-3-yl)benzyl)piperidin-4-yl)-5-fluorobenzo[d]thiazole-2,7-dicarboxamide

Following the general procedure of Example 173 step 1, the reaction of Intermediate 11 (50 mg, 105 μmol) with Intermediate 26 (25.3 mg, 105 μmol) was carried out. After purified by prep-HPLC (column: Welch Xtimate C18 150×25 mm×5 μm; mobile phase: [water (NH₃H₂O)-ACN]; B %: 38%-68%, 8 min), N⁷-(1-(4-(2-(2-aminopyridin-3-yl)-5-phenyl-3H-imidazo[4,5-b]pyridin-3-yl)benzyl)piperidin-4-yl)-5-fluorobenzo[d]thiazole-2,7-dicarboxamide (8.6 mg, yield: 11%) was obtained as pink solid. MS: m/z=698.2 [M+H]⁺. 1H NMR (400 MHz, Dimethylsulfoxide-d₆) δ 8.77 (d, J=8.0 Hz, 1H), 8.46 (s, 1H), 8.29-8.24 (m, 2H), 8.14 (dd, J=8.8, 2.0 Hz, 1H), 8.08 (s, 1H), 8.05-7.97 (m, 4H), 7.53-7.45 (m, 6H), 7.42-7.37 (m, 1H), 7.17 (dd, J=7.6, 2.0 Hz, 1H), 7.04 (br s, 2H), 6.39 (dd, J=7.6, 4.8 Hz, 1H), 3.93-3.85 (m, 1H), 3.61 (s, 2H), 2.95-2.87 (m, 2H), 2.18-2.09 (m, 2H), 1.92-1.85 (m, 2H), 1.72-1.62 (m, 2H). ¹⁹F NMR (400 MHz, Dimethylsulfoxide-d₆) 6-114.6.

### Step 2: N-(1-(4-(2-(2-Aminopyridin-3-yl)-5-phenyl-3H-imidazo[4,5-b]pyridin-3-yl)benzyl)piperidin-4-yl)-2-cyano-5-fluorobenzo[d]thiazole-7-carboxamide

Following the general procedure of Example 173 step 2, the reaction of N⁷-(1-(4-(2-(2-aminopyridin-3-yl)-5-phenyl-3H-imidazo[4,5-b]pyridin-3-yl)benzyl)piperidin-4-yl)-5-fluorobenzo[d]thiazole-2,7-dicarboxamide (20 mg, 29 μmol) was carried out. After purified by prep-HPLC (column: Phenomenex C18 150×25 mm×10 μm; mobile phase: [water (NH₄HCO₃)-ACN]; B %: 50%-80%, 8 min), N-(1-(4-(2-(2-aminopyridin-3-yl)-5-phenyl-3H-imidazo[4,5-b]pyridin-3-yl)benzyl)piperidin-4-yl)-2-cyano-5-fluorobenzo[d]thiazole-7-carboxamide (Example 182, 3.7 mg, yield: 18%) was obtained as off-white lyophilized powder. MS: m/z=680.2 [M+H]⁺. ¹H NMR (400 MHz, Dimethylsulfoxide-d₆) δ ¹H NMR (400 MHz, Dimethylsulfoxide-d₆) δ 9.01 (d, J=6.8 Hz, 1H), 8.41 (d, J=7.2 Hz, 1H), 8.27 (d, J=8.0 Hz, 1H), 8.07-7.90 (m, 6H), 7.54-7.43 (m, 5H), 7.39 (d, J=7.2 Hz, 1H), 7.17 (d, J=6.4 Hz, 1H), 7.00 (br s, 2H), 6.44-6.34 (m, 1H), 4.06-3.94 (m, 2H), 3.64-3.62 (m, 1H), 2.96-2.77 (m, 4H), 2.03-1.92 (m, 2H), 1.79-1.60 (m, 2H). ¹⁹F NMR (400 MHz, Dimethylsulfoxide-d₆) δ −109.8.

### Example 183: N-(1-(4-(2-(2-aminopyridin-3-yl)-5-phenyl-3H-imidazo[4,5-b]pyridin-3-yl)benzyl)piperidin-4-yl)-2-cyano-6-fluorobenzo[d]thiazole-4-carboxamide

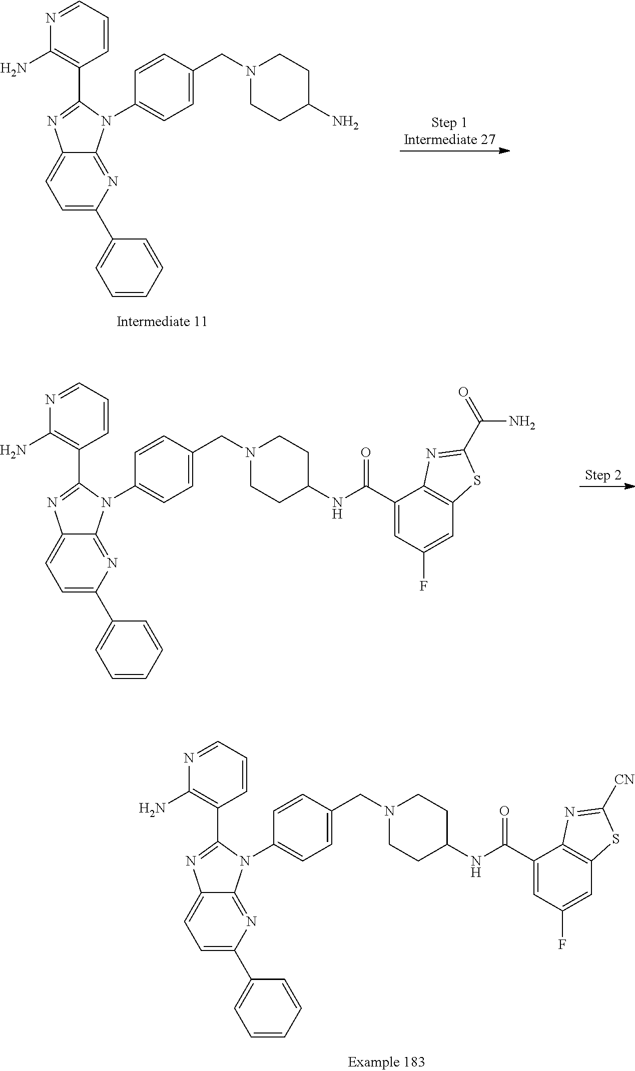 [SMILES: Nc1ncccc1-c1nc2ccc(-c3ccccc3)nc2n1-c1ccc(CN2CCC(N)CC2)cc1 ; NC(=O)c1nc2c(C(=O)NC3CCN(Cc4ccc(-n5c(-c6cccnc6N)nc6ccc(-c7ccccc7)nc65)cc4)CC3)cc(F)cc2s1 ; N#Cc1nc2c(C(=O)NC3CCN(Cc4ccc(-n5c(-c6cccnc6N)nc6ccc(-c7ccccc7)nc65)cc4)CC3)cc(F)cc2s1]

### Step 1: N⁴-(1-(4-(2-(2-aminopyridin-3-yl)-5-phenyl-3H-imidazo[4,5-b]pyridin-3-yl)benzyl)piperidin-4-yl)-6-fluorobenzo[d]thiazole-2,4-dicarboxamide

Following the general procedure of Example 173 step 1, the reaction of Intermediate 11 (50 mg, 105 μmol) with Intermediate 27 (25.2 mg, 105 μmol) was carried out. After purified by prep-HPLC (column: Waters Xbridge BEH C18 100×30 mm×10 μm; mobile phase: [water (ammonia hydroxide v/v)-ACN]; B %: 46%-76%, 2 min), N⁴-(1-(4-(2-(2-aminopyridin-3-yl)-5-phenyl-3H-imidazo[4,5-b]pyridin-3-yl)benzyl)piperidin-4-yl)-6-fluorobenzo[d]thiazole-2,4-dicarboxamide (10.5 mg, yield: 14%) was obtained as a light-yellow lyophilized powder. MS: m/z=698.2 [M+H]⁺. ¹H NMR (400 MHz, Dimethylsulfoxide-d₆) δ 9.20 (d, J=7.6 Hz, 1H), 8.67 (s, 1H), 8.36-8.26 (m, 3H), 8.05-7.97 (m, 5H), 7.53-7.45 (m, 6H), 7.42-7.38 (m, 1H), 7.16 (dd, J=7.6, 1.8 Hz, 1H), 7.04 (br s, 2H), 6.38 (dd, J=7.6, 4.8 Hz, 1H), 4.02-3.89 (m, 1H), 3.60 (s, 2H), 2.95-2.84 (m, 2H), 2.22-2.11 (m, 2H), 1.98-1.84 (m, 4H). ¹⁹F NMR (400 MHz, Dimethylsulfoxide-d₆) δ −112.7.

### Step 2: N-(1-(4-(2-(2-aminopyridin-3-yl)-5-phenyl-3H-imidazo[4,5-b]pyridin-3-yl)benzyl)piperidin-4-yl)-2-cyano-6-fluorobenzo[d]thiazole-4-carboxamide

Following the general procedure of Example 173 step 2, the reaction of N⁴-(1-(4-(2-(2-aminopyridin-3-yl)-5-phenyl-3H-imidazo[4,5-b]pyridin-3-yl)benzyl)piperidin-4-yl)-6-fluorobenzo[d]thiazole-2,4-dicarboxamide (40 mg, 57 μmol) with POCl₃ was carried out. After purified by prep-HPLC (column: Phenomenex C18 150×25 mm×10 μm; mobile phase: [water (NH₄HCO₃)-ACN]; B %: 50%-80%, 8 min), N-(1-(4-(2-(2-aminopyridin-3-yl)-5-phenyl-3H-imidazo[4,5-b]pyridin-3-yl)benzyl)piperidin-4-yl)-2-cyano-6-fluorobenzo[d]thiazole-4-carboxamide (Example 183, 14.6 mg, yield: 37%) was obtained as an off-white yellow lyophilized powder. MS: m/z=680.2 [M+H]⁺. ¹H NMR (400 MHz, Dimethylsulfoxide-d₆) δ 8.97 (d, J=7.6 Hz, 1H), 8.46 (dd, J=9.6, 1.6 Hz, 1H), 8.39-8.33 (m, 1H), 8.25 (d, J 8.4 Hz, 1H), 8.04-7.95 (m, 4H), 7.52-7.48 (m, 2H), 7.48-7.43 (m, 4H), 7.41-7.36 (m, 1H), 7.18-7.12 (m, 1H), 7.03 (br s, 2H), 6.37 (dd, J=7.6, 4.8 Hz, 1H), 3.94-3.82 (m, 1H), 3.61 (s, 2H), 2.96-2.85 (m, 2H), 2.19-2.08 (m, 2H), 1.93-1.83 (m, 2H), 1.74-1.61 (m, 2H). ¹⁹F NMR (400 MHz, Dimethylsulfoxide-d₆) δ −113.04.

### Example 184: 5-(4-(4-(2-(2-aminopyridin-3-yl)-5-phenyl-3H-imidazo[4,5-b]pyridin-3-yl)benzyl)piperazine-1-carbonyl)benzo[d]thiazole-2-carbonitrile

### Step 1: 5-(4-(4-(2-(2-Aminopyridin-3-yl)-5-phenyl-3H-imidazo[4,5-b]pyridin-3-yl)benzyl)piperazine-1-carbonyl)benzo[d]thiazole-2-carboxamide

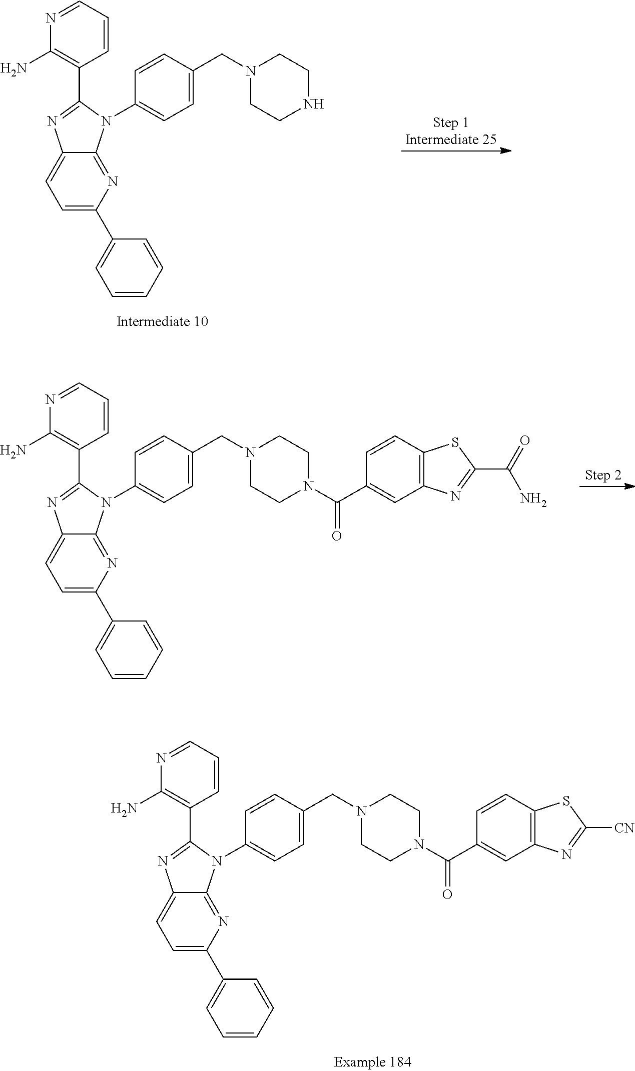 [SMILES: Nc1ncccc1-c1nc2ccc(-c3ccccc3)nc2n1-c1ccc(CN2CCNCC2)cc1 ; NC(=O)c1nc2cc(C(=O)N3CCN(Cc4ccc(-n5c(-c6cccnc6N)nc6ccc(-c7ccccc7)nc65)cc4)CC3)ccc2s1 ; N#Cc1nc2cc(C(=O)N3CCN(Cc4ccc(-n5c(-c6cccnc6N)nc6ccc(-c7ccccc7)nc65)cc4)CC3)ccc2s1]

Following the general procedure of Example 173 step 1, the reaction of Intermediate 10 (360 mg, 778 μmol) with Intermediate 25 (208 mg, 934 μmol) was carried out. After purified by prep-HPLC (column: Waters Xbridge 150*25 mm*5 μm; mobile phase: [water (NH₄HCO₃)-ACN]; B %: 34%-64%, 8 min), 5-(4-(4-(2-(2-aminopyridin-3-yl)-5-phenyl-3H-imidazo[4,5-b]pyridin-3-yl)benzyl)piperazine-1-carbonyl)benzo[d]thiazole-2-carboxamide (64.7 mg, yield: 12%) was obtained as a yellow solid. MS: m/z=666.1 [M+H]⁺. ¹H NMR (400 MHz, Chlorform-d) δ 8.18-8.10 (m, 2H), 8.08-7.96 (m, 3H), 7.80 (d, J=8.4 Hz, 1H), 7.58 (dd, J=8.4, 1.2 Hz, 1H), 7.50 (d, J=8.0 Hz, 2H), 7.45-7.39 (m, 4H), 7.38-7.34 (m, 1H), 7.30 (s, 1H), 7.09 (dd, J=7.6, 1.6 Hz, 1H), 6.64 (br s, 2H), 6.35 (dd, J=7.6, 4.8 Hz, 1H), 5.76 (s, 1H), 4.03-3.77 (m, 2H), 3.68 (s, 2H), 3.62-3.41 (m, 2H), 2.71-2.43 (m, 4H).

### Step 2: 5-(4-(4-(2-(2-Aminopyridin-3-yl)-5-phenyl-3H-imidazo[4,5-b]pyridin-3-yl)benzyl)piperazine-1-carbonyl)benzo[d]thiazole-2-carbonitrile

Following the general procedure of Example 173 step 2, the reaction of 5-(4-(4-(2-(2-aminopyridin-3-yl)-5-phenyl-3H-imidazo[4,5-b]pyridin-3-yl)benzyl)piperazine-1-carbonyl)benzo[d]thiazole-2-carboxamide (50 mg, 45 μmol) with POCl₃ (173 mg, 1.0 mmol) was carried out. The crude product was purified by reversed-phase HPLC (0.1% NH; HCO₃), 5-(4-(4-(2-(2-aminopyridin-3-yl)-5-phenyl-3H-imidazo[4,5-b]pyridin-3-yl)benzyl)piperazine-1-carbonyl)benzo[d]thiazole-2-carbonitrile (Example 184, 4.5 mg, yield: 15%) was obtained as a light-green solid. MS: m/z=648.4 [M+H]⁺. ¹H NMR (400 MHz, Chlorform-d) δ 8.26 (s, 1H), 8.14 (d, J=8.4 Hz, 1H), 8.07-8.03 (m, 2H), 8.00 (d, J=7.2 Hz, 2H), 7.81 (d, J=8.4 Hz, 1H), 7.72 (dd, J=8.4, 1.2 Hz, 1H), 7.51 (d, J=8.0 Hz, 2H), 7.46-7.40 (m, 4H), 7.38 (d, J=7.2 Hz, 1H), 7.13-7.09 (m, 1H), 6.83 (br s, 2H), 6.36 (dd, J=7.6, 5.2 Hz, 1H), 4.00-3.82 (m, 2H), 3.67 (s, 2H), 3.60-3.43 (m, 2H), 2.69-2.47 (m, 4H).

### Example 185: N-(1-(4-(2-(2-aminopyridin-3-yl)-5-phenyl-3H-imidazo[4,5-b]pyridin-3-yl)benzyl)piperidin-4-yl)-2-cyanobenzo[d]thiazole-5-carboxamide

### Step 1: N⁵-(1-(4-(2-(2-Aminopyridin-3-yl)-5-phenyl-3H-imidazo[4,5-b]pyridin-3-yl)benzyl)piperidin-4-yl)benzo[d]thiazole-2,5-dicarboxamide

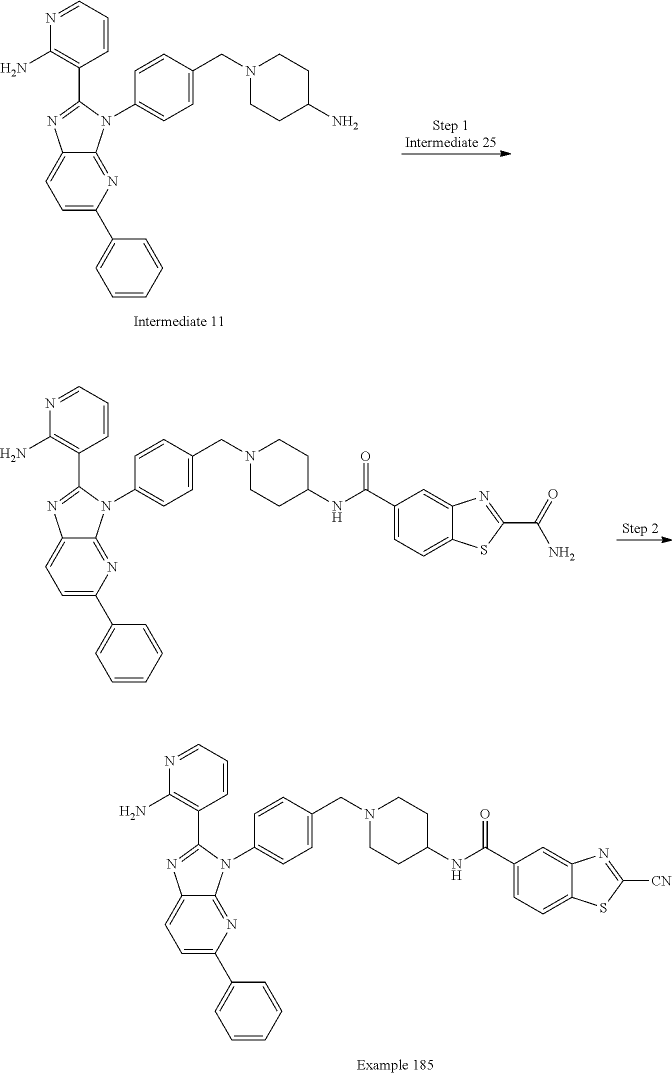 [SMILES: Nc1ncccc1-c1nc2ccc(-c3ccccc3)nc2n1-c1ccc(CN2CCC(N)CC2)cc1 ; NC(=O)c1nc2cc(C(=O)NC3CCN(Cc4ccc(-n5c(-c6cccnc6N)nc6ccc(-c7ccccc7)nc65)cc4)CC3)ccc2s1 ; N#Cc1nc2cc(C(=O)NC3CCN(Cc4ccc(-n5c(-c6cccnc6N)nc6ccc(-c7ccccc7)nc65)cc4)CC3)ccc2s1]

Following the general procedure of Example 173 step 1, the reaction of Intermediate 11 (370 mg, 778 μmol) with Intermediate 25 (208 mg, 934 μmol) was carried out. After purified by prep-HPLC (column: Phenomenex C18 150*25 mm*10 μm; mobile phase: [water (NH₄HCO₃)-ACN]; B %: 33%-63%, 8 min), NM-(1-(4-(2-(2-aminopyridin-3-yl)-5-phenyl-3H-imidazo[4,5-b]pyridin-3-yl)benzyl)piperidin-4-yl)benzo[d]thiazole-2,5-dicarboxamide (39.7 mg, yield: 7.5%) was obtained as a yellow solid. MS: m/z=680.2 [M+H]⁺. ¹H NMR (400 MHz, Chlorform-d) δ 8.46 (d, J=1.2 Hz, 1H), 8.13 (d, J=8.4 Hz, 1H), 8.07-7.93 (m, 5H), 7.81 (d, J=8.4 Hz, 1H), 7.51 (d, J=8.0 Hz, 2H), 7.46-7.40 (m, 4H), 7.39-7.35 (m, 1H), 7.31 (br s, 1H), 7.10 (dd, J=7.6, 1.6 Hz, 1H), 6.61 (br s, 2H), 6.36 (dd, J=7.6, 4.8 Hz, 1H), 6.16 (d, J=7.6 Hz, 1H), 5.78 (s, 1H), 4.15-4.07 (m, 1H), 3.66 (s, 2H), 2.97 (d, J=11.6 Hz, 2H), 2.31 (t, J=10.8 Hz, 2H), 2.13 (d, J=10.0 Hz, 2H), 1.74-1.68 (m, 2H).

### Step 2: N-(1-(4-(2-(2-aminopyridin-3-yl)-5-phenyl-3H-imidazo[4,5-b]pyridin-3-yl)benzyl)piperidin-4-yl)-2-cyanobenzo[d]thiazole-5-carboxamide

Following the general procedure of Example 173 step 2, the reaction of N⁵-(1-(4-(2-(2-aminopyridin-3-yl)-5-phenyl-3H-imidazo[4,5-b]pyridin-3-yl)benzyl)piperidin-4-yl)benzo[d]thiazole-2,5-dicarboxamide (24 mg, 35 μmol) with POCl₃ (133 mg, 865 μmol) was carried out. The crude product was purified by reversed-phase HPLC (0.1% NH₃HCO₃), N-(1-(4-(2-(2-aminopyridin-3-yl)-5-phenyl-3H-imidazo[4,5-b]pyridin-3-yl)benzyl)piperidin-4-yl)-2-cyanobenzo[d]thiazole-5-carboxamide (Example 185, 3.9 mg, yield: 17%) was obtained as a yellow solid. MS: m/z=662.5 [M+H]⁺. ¹H NMR (400 MHz, Chlorform-d) δ 8.55 (s, 1H), 8.14 (d, J=8.4 Hz, 1H), 8.10-8.05 (m, 3H), 8.02 (d, J=7.2 Hz, 2H), 7.81 (d, J=8.4 Hz, 1H), 7.57-7.51 (m, 2H), 7.47-7.35 (m, 5H), 7.10 (dd, J=7.6, 1.6 Hz, 1H), 6.61 (br s, 2H), 6.37 (dd, J=7.6, 4.8 Hz, 1H), 6.28-6.13 (m, 1H), 4.19-4.08 (m, 1H), 3.71 (s, 2H), 3.10-2.95 (m, 2H), 2.42-2.31 (m, 2H), 2.17-2.11 (m, 2H), 1.83-1.74 (m, 2H).

### Example 186: 6-((4-(2-(2-Aminopyridin-3-yl)-5-phenyl-3H-imidazo[4,5-b]pyridin-3-yl)benzyl)amino)picolinonitrile

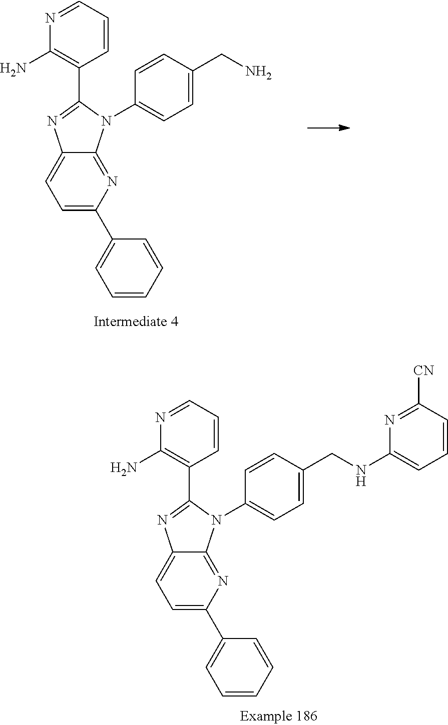 [SMILES: NCc1ccc(-n2c(-c3cccnc3N)nc3ccc(-c4ccccc4)nc32)cc1 ; N#Cc1cccc(NCc2ccc(-n3c(-c4cccnc4N)nc4ccc(-c5ccccc5)nc43)cc2)n1]

To a solution of Intermediate 4 (100 mg, 255 μmol) and 6-fluoropyridine-2-carbonitrile (34.2 mg, 280 μmol) in NMP (2 mL) was added DIEA (164 mg, 1.27 mmol). The mixture was stirred under microwave at 135° C. for 1 hr. The reaction was filtered and concentrated under reduced pressure to give a residue. The crude product was purified by prep-HPLC (column: Phenomenex C18 150*25 mm*10 μm; mobile phase: [water (NH₄HCO₃)-ACN]; B %: 47%-77%, 8 min), 6-((4-(2-(2-aminopyridin-3-yl)-5-phenyl-3H-imidazo[4,5-b]pyridin-3-yl)benzyl)amino)picolinonitrile (Example 186, 53 mg, yield: 42%) was obtained as a light-yellow solid. MS: m/z=495.1 [M+H]⁺. ¹H NMR (400 MHz, Dimethylsulfoxide-d₆) δ 8.27 (d, J=8 Hz, 1H), 8.04-7.98 (m, 4H), 7.79 (t, J=6.0 Hz, 1H), 7.58 (dd, J=8.8, 7.2 Hz, 1H), 7.52-7.45 (m, 6H), 7.41-7.38 (m, 1H), 7.2 (dd, 1=7.6, 1.6 Hz, 1H), 7.12 (d, J=6.4 Hz, 1H), 6.99 (br s, 2H), 6.88 (d, J=8.4 Hz, 1H), 6.40 (dd, J=7.6, 4.8 Hz, 1H), 4.6 (d, J=6.0 Hz, 2H).

### Example 187: 5-((4-(2-(2-Aminopyridin-3-yl)-5-phenyl-3H-imidazo[4,5-b]pyridin-3-yl)benzyl)amino)picolinonitrile

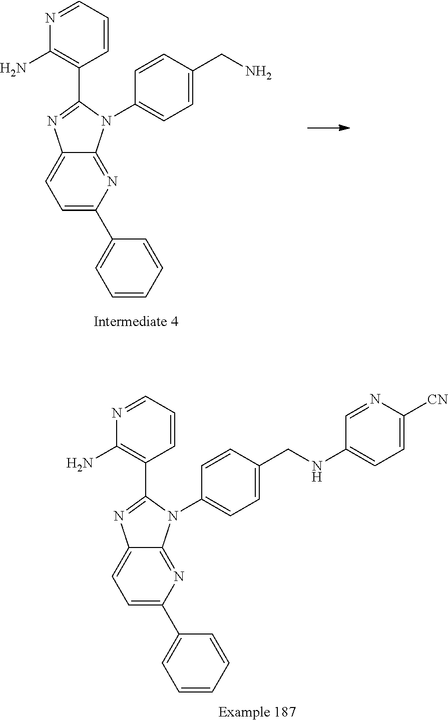 [SMILES: NCc1ccc(-n2c(-c3cccnc3N)nc3ccc(-c4ccccc4)nc32)cc1 ; N#Cc1ccc(NCc2ccc(-n3c(-c4cccnc4N)nc4ccc(-c5ccccc5)nc43)cc2)cn1]

To a solution of Intermediate 4 (150 mg, 382 μmol) and 5-fluoropyridine-2-carbonitrile (51.3 mg, 420 μmol) in DMSO (3 mL) was added DIEA (247 mg, 1.91 mmol). The mixture was stirred at 100° C. for 12 hr. The reaction was filtered and concentrated under reduced pressure to give a residue. The crude product was purified by prep-HPLC (column: Waters xbridge 150*25 mm 10 μm; mobile phase: [water (NH₄HCO₃)-ACN]; B %: 36%-66%, 8 min), 5-((4-(2-(2-aminopyridin-3-yl)-5-phenyl-3H-imidazo[4,5-b]pyridin-3-yl)benzyl)amino)picolinonitrile (Example 187, 22 mg, yield: 11.6%) was obtained as a light-yellow solid. MS: m/z=495.1 [M+H]⁺. ¹H NMR (400 MHz, Dimethylsulfoxide-d₆) δ 8.26 (d, J=8.4 Hz, 1H), 8.14 (d, J=2.4 Hz, 1H), 8.02-7.97 (m, 4H), 7.67-7.61 (m, 2H), 7.54-7.39 (m, 7H), 7.16 (dd, J=7.6, 2 Hz, 1H), 7.02-7.01 (m, 1H), 6.8 (br s, 2H), 6.37 (dd, J=7.6, 4.8 Hz, 1H), 4.51 (d, J=6.0 Hz, 2H).

### Example 188: 5-((4-(2-(2-Aminopyridin-3-yl)-5-phenyl-3H-imidazo[4,5-b]pyridin-3-yl)benzyl)amino)nicotinonitrile

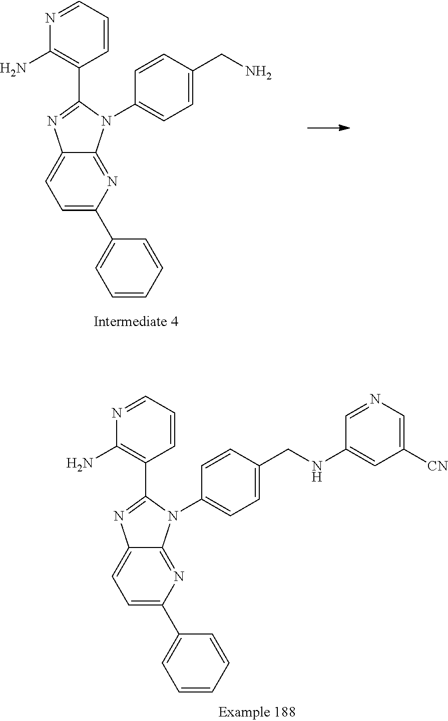 [SMILES: NCc1ccc(-n2c(-c3cccnc3N)nc3ccc(-c4ccccc4)nc32)cc1 ; N#Cc1cncc(NCc2ccc(-n3c(-c4cccnc4N)nc4ccc(-c5ccccc5)nc43)cc2)c1]

To a solution of Intermediate 4 (150 mg, 382 μmol) and 5-fluoropyridine-3-carbonitrile (56.0 mg, 459 μmol) in DMSO (3 mL) was added DIEA (247 mg, 1.91 mmol). The mixture was stirred at 100° C. for 12 hr. The reaction was filtered and concentrated under reduced pressure to give a residue. The crude product was purified by prep-HPLC (column: Phenomenex C18 150*25 mm*10 μm; mobile phase: [water (NH₄HCO₃)-ACN]; B %: 41%-71%, 8 min), 5-((4-(2-(2-aminopyridin-3-yl)-5-phenyl-3H-imidazo[4,5-b]pyridin-3-yl)benzyl)amino)nicotinonitrile (Example 188, 7.0 mg, yield: 3.7%) was obtained as a yellow solid. MS: m/z=495.5 [M+H]⁺. ¹H NMR (400 MHz, Dimethylsulfoxide-d₆) δ 8.31 (d, J=2.8 Hz, 1H), 8.26 (d, J=8.4 Hz, 1H), 8.14 (d, J=2.0 Hz, 1H), 8.03-7.97 (m, 4H), 7.54 (d, J=8.4 Hz, 2H), 7.49-7.37 (m, 5H), 7.32-7.24 (m, 1H), 7.19-7.14 (m, 2H), 7.01 (br s, 2H), 6.36 (dd, J=7.6, 4.8 Hz, 1H), 4.47 (d, J=6.0 Hz, 1H).

### Example 189:2-((4-(2-(2-Aminopyridin-3-yl)-5-phenyl-3H-imidazo[4,5-b]pyridin-3-yl)benzyl)amino)isonicotinonitrile

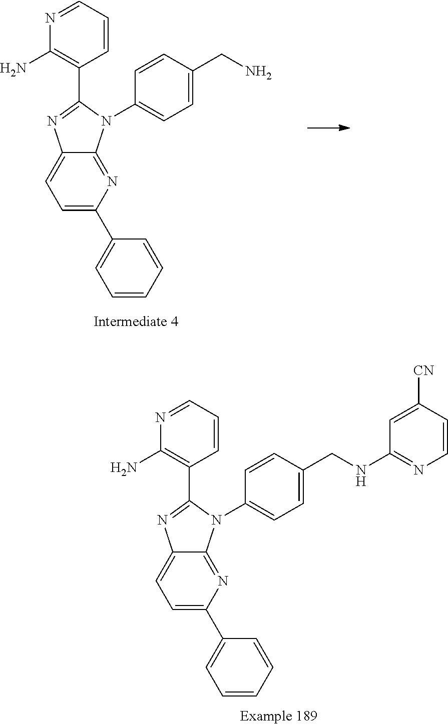 [SMILES: NCc1ccc(-n2c(-c3cccnc3N)nc3ccc(-c4ccccc4)nc32)cc1 ; N#Cc1ccnc(NCc2ccc(-n3c(-c4cccnc4N)nc4ccc(-c5ccccc5)nc43)cc2)c1]

To a solution of Intermediate 4 (150 mg, 382 μmol) and 2-chloropyridine-4-carbonitrile (58.3 mg, 420 μmol) in NMP (3 mL) was added DIEA (247 mg, 1.91 mmol). The mixture was stirred under microwave at 160° C. for 1 hr. The reaction was filtered and concentrated under reduced pressure to give a residue. The crude product was purified by prep-HPLC (column: Waters xbridge 150*25 mm 10 μm; mobile phase: [water (NH₄HCO₃)-ACN]; B %: 55%-85%, 5 min), 2-((4-(2-(2-aminopyridin-3-yl)-5-phenyl-3H-imidazo[4,5-b]pyridin-3-yl)benzyl)amino)isonicotinonitrile (Example 189, 4.4 mg, yield: 2.3%) was obtained as a light yellow solid. MS: m/z=495.2 [M+H]⁺. ¹H NMR (400 MHz, Dimethylsulfoxide-d₆) δ 8.26 (d, J=8.4 Hz, 1H), 8.19 (d, J=5.2 Hz, 1H), 8.03-7.97 (m, 4H), 7.75-7.73 (m, 1H), 7.50-7.42 (m, 6H), 7.41-7.39 (m, 1H), 7.19 (dd, J=7.6, 1.6 Hz, 1H), 6.97 (br s, 2H), 6.91 (s, 1H), 6.84 (dd, J=5.2, 1.2 Hz, 1H), 6.39 (dd, J=7.6, 4.8 Hz, 1H), 4.63 (d, J=5.6 Hz, 2H).

### Example 190: 6-((4-(2-(2-Aminopyridin-3-yl)-5-phenyl-3H-imidazo[4,5-b]pyridin-3-yl)benzyl)amino)pyrazine-2-carbonitrile

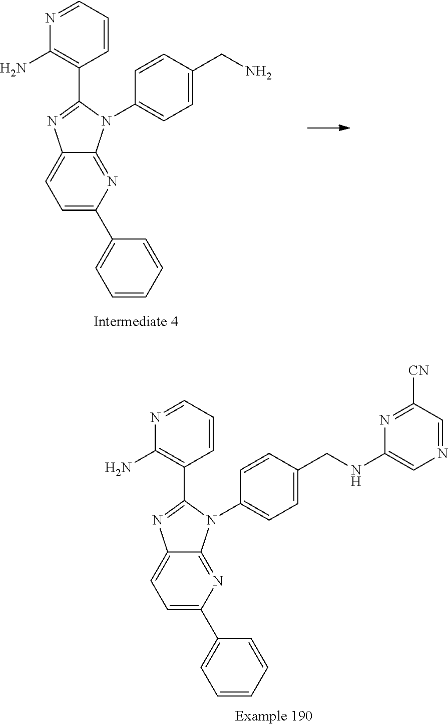 [SMILES: NCc1ccc(-n2c(-c3cccnc3N)nc3ccc(-c4ccccc4)nc32)cc1 ; N#Cc1cncc(NCc2ccc(-n3c(-c4cccnc4N)nc4ccc(-c5ccccc5)nc43)cc2)n1]

To a solution of Intermediate 4 (150 mg, 382 μmol) and 6-chloropyrazine-2-carbonitrile (58.7 mg, 420 μmol) in NMP (3 mL) was added DIEA (247 mg, 1.91 mmol). The mixture was stirred under microwave at 135° C. for 1 hr. The reaction was filtered and concentrated under reduced pressure to give a residue. The crude product was purified by prep-HPLC (column: Waters xbridge 150*25 mm 10 μm; mobile phase: [water (NH₄HCO₃)-ACN]; B %: 19%-49%, 9 min), 6-((4-(2-(2-aminopyridin-3-yl)-5-phenyl-3H-imidazo[4,5-b]pyridin-3-yl)benzyl)amino)pyrazine-2-carbonitrile (Example 190, 6.0 mg, yield: 3.2%) was obtained as a brown solid. MS: m/z=496.3 [M+H]⁺. ¹H NMR (400 MHz, Dimethylsulfoxide-d₆) δ 8.37 (t, J=6.0 Hz, 1H), 8.29-8.22 (m, 3H), 8.03-7.97 (m, 4H), 7.53-7.51 (m, 2H), 7.49-7.44 (m, 4H), 7.41-7.37 (m, 1H), 7.20 (dd, J=7.6, 1.6 Hz, 1H), 6.96 (br s, 2H), 6.4 (dd, J=7.6, 4.8 Hz, 1H), 4.60 (d, J=6.0 Hz, 2H).

### Example 191: 2-((4-(2-(2-Aminopyridin-3-yl)-5-phenyl-3H-imidazo[4,5-b]pyridin-3-yl)benzyl)amino)pyrimidine-4-carbonitrile

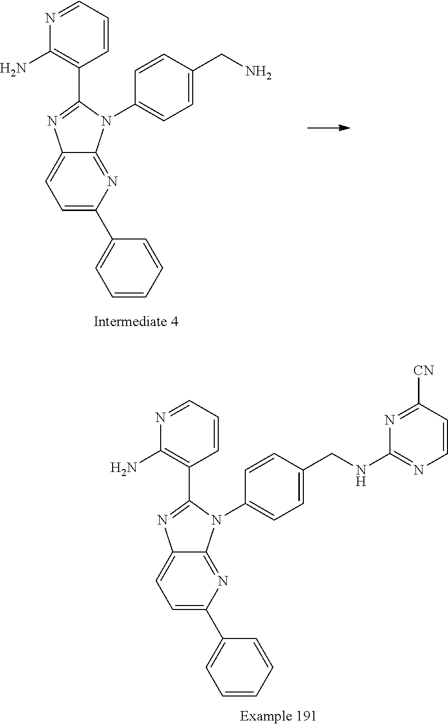 [SMILES: NCc1ccc(-n2c(-c3cccnc3N)nc3ccc(-c4ccccc4)nc32)cc1 ; N#Cc1ccnc(NCc2ccc(-n3c(-c4cccnc4N)nc4ccc(-c5ccccc5)nc43)cc2)n1]

To a solution of Intermediate 4 (150 mg, 382 μmol) and 2-chloropyrimidine-4-carbonitrile (58.7 mg, 420 μmol) in NMP (3 mL) was added DIEA (247 mg, 1.91 mmol). The mixture was stirred under microwave at 135° C. for 1 hr. The reaction was filtered and concentrated under reduced pressure to give a residue. The crude product was purified by prep-HPLC (column: Waters xbridge 150*25 mm 10 μm; mobile phase: [water (NH₄HCO₃)-ACN]; B %: 39%-69%, 8 min), 2-((4-(2-(2-aminopyridin-3-yl)-5-phenyl-3H-imidazo[4,5-b]pyridin-3-yl)benzyl)amino)pyrimidine-4-carbonitrile (Example 191, 7.4 mg, yield: 3.9%) was obtained as a brown solid. MS: m/z=496.2 [M+H]⁺. ¹H NMR (400 MHz, Dimethylsulfoxide-d₆) δ 8.60 (d, J=4.4 Hz, 1H), 8.51-8.48 (m, 1H), 8.26 (d, J=8.4 Hz, 1H), 8.03-7.97 (m, 4H), 7.48-7.44 (m, 6H), 7.41-7.37 (m, 1H), 7.19-7.17 (m, 1H), 7.15 (d, J=4.8 Hz, 1H), 6.97 (br s, 2H), 6.39 (dd, J=7.6, 4.8 Hz, 1H), 4.66-4.58 (br s, 2H).

### Example 192: 4-((2S,6S)-4-(4-(2-(2-Aminopyridin-3-yl)-5-phenyl-3H-imidazo[4,5-b]pyridin-3-yl)benzyl)-2,6-dimethylpiperazin-1- yl)pyrimidine-2-carbonitrile

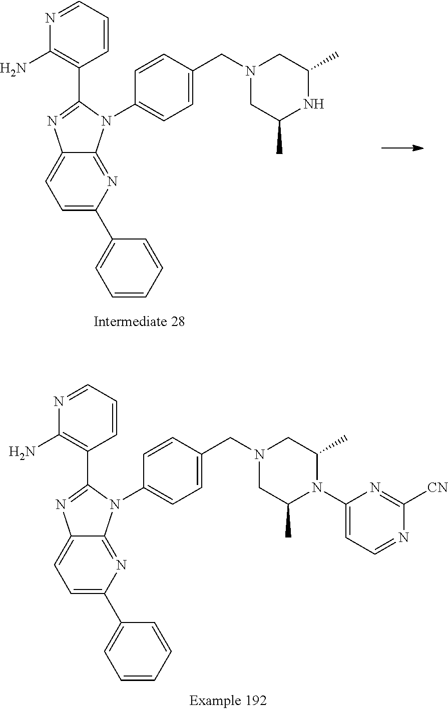 [SMILES: C[C@H]1CN(Cc2ccc(-n3c(-c4cccnc4N)nc4ccc(-c5ccccc5)nc43)cc2)C[C@H](C)N1 ; C[C@H]1CN(Cc2ccc(-n3c(-c4cccnc4N)nc4ccc(-c5ccccc5)nc43)cc2)C[C@H](C)N1c1ccnc(C#N)n1]

Intermediate 28 (100 mg, 204 μmol), 4-chloropyrimidine-2-carbonitrile (28.5 mg, 204 μmol) and DIEA (106 mg, 817 μmol) were taken up into a microwave tube in NMP (2 mL). The sealed tube was heated at 160° C. for 1 hr under microwave. The reaction mixture was quenched with H₂O (5 mL) at 0° C., and then diluted with CH₂Cl₂ (10 mL) and extracted with CH₂Cl₂ 30 mL (15 mL×2). The combined organic layers were washed with 15 mL aqueous NaCl, dried over Na₂SO₄, filtered and concentrated under reduced pressure to give a residue. After purified by prep-HPLC (column: Welch Xtimate C18 150×25 mm 5 μm; mobile phase: [water (NH₄HCO₃)-ACN]; B %: 60%-90%, 20 min), 4-((2S,6S)-4-(4-(2-(2-aminopyridin-3-yl)-5-phenyl-3H-imidazo[4,5-b]pyridin-3-yl)benzyl)-2,6-dimethylpiperazin-1-yl)pyrimidine-2-carbonitrile (Example 192, 10.5 mg, yield. 8.3%) was obtained as a brown lyophilized powder. MS: m/z=593.3 [M+H]⁺. ¹H NMR (400 MHz, Dimethylsulfoxide-d₆) 8.33-8.26 (m, 2H), 8.05-7.98 (m, 4H), 7.55-7.52 (m, 2H), 7.51-7.36 (m, 6H), 7.18 (dd, J=7.6, 1.6 Hz, 1H), 7.04 (br s, 2H), 6.39 (dd, J=7.6, 4.8 Hz, 1H), 4.24-4.18 (m, 2H), 3.94 (d, J=13.6 Hz, 1H), 3.80 (d, J=13.6 Hz, 1H), 3.11 (dd, J=12.0, 4.0 Hz, 2H), 2.68 (dd, J=12.0, 3.6 Hz, 2H), 1.27 (d, J=6.4 Hz, 6H).

### Example 193: 2-((2S,6S)-4-(4-(2-(2-Aminopyridin-3-yl)-5-phenyl-3H-imidazo[4,5-b]pyridin-3-yl)benzyl)-2,6-dimethylpiperazin-1-yl)pyrimidine-4-carbonitrile

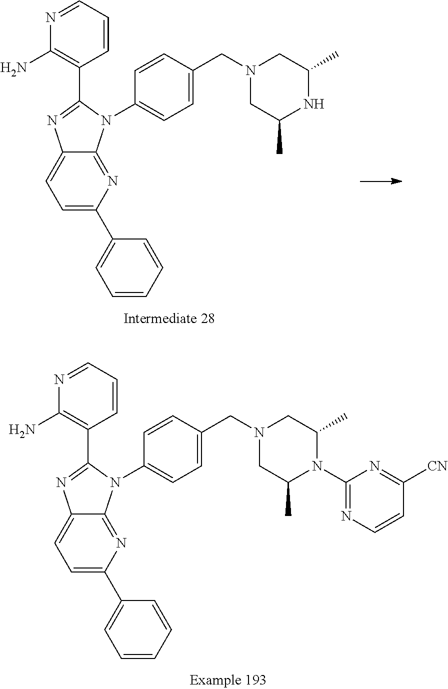 [SMILES: C[C@H]1CN(Cc2ccc(-n3c(-c4cccnc4N)nc4ccc(-c5ccccc5)nc43)cc2)C[C@H](C)N1 ; C[C@H]1CN(Cc2ccc(-n3c(-c4cccnc4N)nc4ccc(-c5ccccc5)nc43)cc2)C[C@H](C)N1c1nccc(C#N)n1]

Intermediate 28 (100 mg, 204 μmol), 2-chloropyrimidine-4-carbonitrile (28.5 mg, 204 μmol) and DIEA (106 mg, 817 μmol) were taken up into a microwave tube in NMP (2 mL). The sealed tube was heated at 160° C. for 1 hr under microwave. The reaction mixture was quenched with H₂O (5 mL) at 0° C., and then diluted with CH₂Cl₂ (10 mL) and extracted with CH₂Cl₂ (15 mL×2). The combined organic layers were washed with 15 mL aqueous NaCl, dried over Na₂SO₄, filtered and concentrated under reduced pressure to give a residue. After purified by prep-HPLC (column: Welch Xtimate C18 150×25 mm 5 μm; mobile phase: [water (NH₄HCO₃)-ACN]; B %: 60%-90%, 20 min), 2-((2S,6S)-4-(4-(2-(2-aminopyridin-3-yl)-5-phenyl-3H-imidazo[4,5-b]pyridin-3-yl)benzyl)-2,6-dimethylpiperazin-1-yl)pyrimidine-4-carbonitrile (Example 193, 6.1 mg, yield: 4.9%) was obtained as a brown lyophilized powder. MS: m/z=593.5 [M+H]⁺. ¹H NMR (400 MHz, Dimethylsulfoxide-d₆) 8.72 (d, J=4.4 Hz, 1H), 8.27 (d, J=8.4 Hz, 1H), 8.05-7.97 (m, 4H), 7.56-7.52 (m, 2H), 7.49-7.37 (m, 5H), 7.25 (d, J=4.8 Hz, 1H), 7.17 (dd, J=7.6, 1.6 Hz, 1H), 7.05 (br s, 2H), 6.39 (dd, J=7.6, 4.8 Hz, 1H), 4.33-4.26 (m, 2H), 3.87 (d, J=13.6 Hz, 1H), 3.69 (d, J=13.6 Hz, 1H), 2.95 (dd, J=11.6, 3.6 Hz, 2H), 2.56 (dd, J=12.0, 4.8 Hz, 2H), 1.26 (d, J=6.4 Hz, 6H).

### Example 194: 6-((2S,6S)-4-(4-(2-(2-Aminopyridin-3-yl)-5-phenyl-3H-imidazo[4,5-b]pyridin-3-yl)benzyl)-2,6-dimethylpiperazin-1-yl)pyrimidine-4-carbonitrile

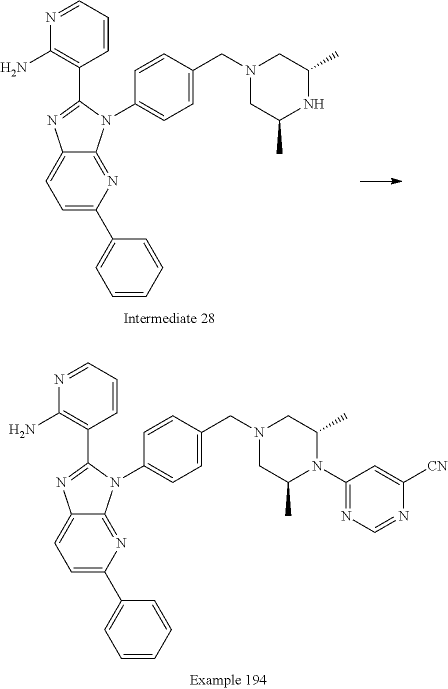 [SMILES: C[C@H]1CN(Cc2ccc(-n3c(-c4cccnc4N)nc4ccc(-c5ccccc5)nc43)cc2)C[C@H](C)N1 ; C[C@H]1CN(Cc2ccc(-n3c(-c4cccnc4N)nc4ccc(-c5ccccc5)nc43)cc2)C[C@H](C)N1c1cc(C#N)ncn1]

Intermediate 28 (100 mg, 204 μmol), 6-chloropyrimidine-4-carbonitrile (28.5 mg, 204 μmol) and DIEA (106 mg, 817 μmol) were taken up into a microwave tube in NMP (2 mL). The sealed tube was heated at 160° C. for 1 hr under microwave. The reaction mixture was quenched with H₂O (5 mL) at 0° C., and then diluted with 10 mL CH₂Cl₂ (10 mL) and extracted with CH₂Cl₂ 30 mL (15 mL×2). The combined organic layers were washed with 15 mL aqueous NaCl, dried over Na₂SO₄, filtered, and concentrated under reduced pressure to give a residue. After purified by prep-HPLC (column: Welch Xtimate C18 150×25 mm 5 μm; mobile phase: [water (NH₄HCO₃)-ACN]; B %: 55%-85%, 20 min), 6-((2S,6S)-4-(4-(2-(2-aminopyridin-3-yl)-5-phenyl-3H-imidazo[4,5-b]pyridin-3-yl)benzyl)-2,6-dimethylpiperazin-1-yl)pyrimidine-4-carbonitrile (Example 194, 8.5 mg, yield: 6.9%) was obtained as a brown lyophilized powder. MS: m/z=593.3 [M+H]⁺. ¹H NMR (400 MHz, Dimethylsulfoxide-d₆) δ 8.64 (s, 1H), 8.27 (d, J=8.4 Hz, 1H), 8.05-7.97 (m, 4H), 7.56-7.52 (m, 2H), 7.49-7.44 (m, 5H), 7.42-7.37 (m, 1H), 7.17 (dd, J=7.6, 1.6 Hz, 1H), 7.04 (br s, 2H), 6.38 (dd, J=7.6, 4.8 Hz, 1H), 4.29-4.22 (m, 2H), 3.95 (d, J=14.0 Hz, 1H), 3.82 (d, J=13.6 Hz, 1H), 3.14 (dd, J=12.0, 4.0 Hz, 2H), 2.69 (dd, J=12.0, 3.2 Hz, 2H), 1.27 (d, J=6.4 Hz, 6H).

### Example 195: 4-((2R,6R)-4-(4-(2-(2-aminopyridin-3-yl)-5-phenyl-3H-imidazo[4,5-b]pyridin-3-yl)benzyl)-2,6-dimethylpiperazin-1-yl)pyrimidine-2-carbonitrile

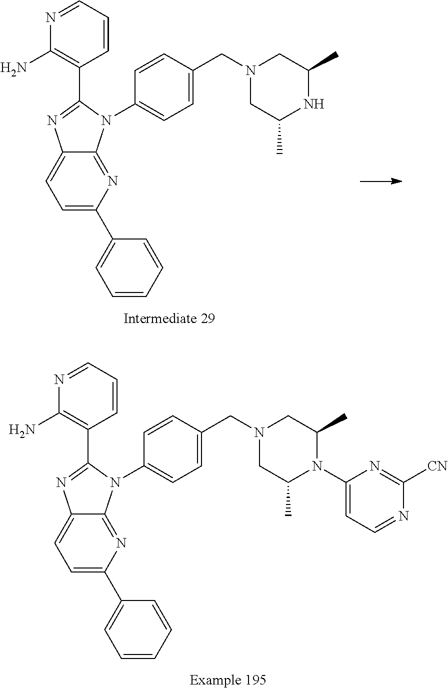 [SMILES: C[C@@H]1CN(Cc2ccc(-n3c(-c4cccnc4N)nc4ccc(-c5ccccc5)nc43)cc2)C[C@@H](C)N1 ; C[C@@H]1CN(Cc2ccc(-n3c(-c4cccnc4N)nc4ccc(-c5ccccc5)nc43)cc2)C[C@@H](C)N1c1ccnc(C#N)n1]

To a solution of 4-chloropyrimidine-2-carbonitrile (31 mg, 225 μmol) in NMP (2 mL) was added DIEA (105 mg, 817 μmol) and Intermediate 29 (100 mg, 204 μmol). The mixture was heated under microwave and stirred at 160° C. for 1 hr, the reaction mixture was filtered to get a liquid. The liquid was purified by prep-HPLC (column: Phenomenex C18 150*25 mm*10 μm; mobile phase: [water (NH₄HCO₃)-ACN]; B %: 50%-80%, 14 min), 4-((2R,6R)-4-(4-(2-(2-aminopyridin-3-yl)-5-phenyl-3H-imidazo[4,5-b]pyridin-3-yl)benzyl)-2,6-dimethylpiperazin-1-yl)pyrimidine-2-carbonitrile (Example 195, 18.3 mg, yield: 12.3%) was obtained as a white lyophilized powder. MS: m/z=593.4 [M+H]⁺. ¹H NMR (400 MHz, Methanol-d₄) δ 8.25-8.13 (m, 2H), 8.03 (d, J=7.2 Hz, 2H), 7.98 (dd, J=5.2, 1.6 Hz, 1H), 7.93 (d, J=8.4 Hz, 1H), 7.56 (d, J=8.4 Hz, 2H), 7.46-7.40 (m, 4H), 7.37 (d, J=7.2 Hz, 1H), 7.32 (dd, J=7.6, 1.6 Hz, 1H), 6.91 (d, J=6.4 Hz, 1H), 6.47 (dd, J=7.6, 5.2 Hz, 1H), 4.32-4.19 (m, 2H), 3.94 (d, J=13.6 Hz, 1H), 3.81 (m, J=13.6 Hz, 1H), 3.20-3.12 (m, 2H), 2.78-2.70 m, 2H), 1.36 (d, J=6.4 Hz, 6H).

### Example 196: 2-((2R,6R)-4-(4-(2-(2-aminopyridin-3-yl)-5-phenyl-3H-imidazo[4,5-b]pyridin-3-yl)benzyl)-2,6-dimethylpiperazin-1-yl)pyrimidine-4-carbonitrile

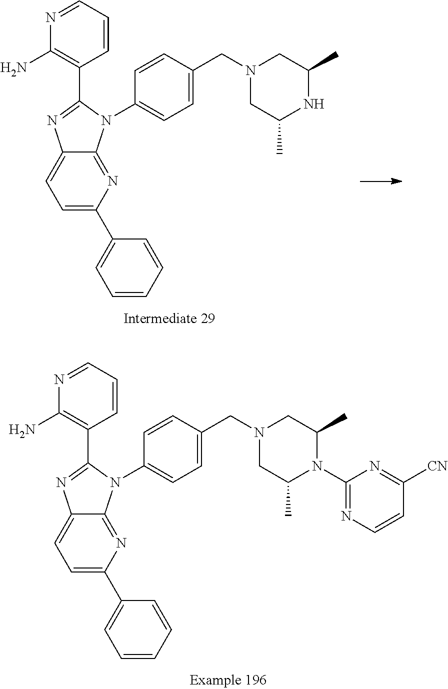 [SMILES: C[C@@H]1CN(Cc2ccc(-n3c(-c4cccnc4N)nc4ccc(-c5ccccc5)nc43)cc2)C[C@@H](C)N1 ; C[C@@H]1CN(Cc2ccc(-n3c(-c4cccnc4N)nc4ccc(-c5ccccc5)nc43)cc2)C[C@@H](C)N1c1nccc(C#N)n1]

To a solution of 2-chloropyrimidine-4-carbonitrile (31 mg, 225 μmol) in NMP (2 mL) was added DIEA (106 mg, 817 μmol) and Intermediate 29 (100 mg, 204 μmol). The mixture was heated under microwave irradiation and was stirred at 160° C. for 1 hr. The reaction mixture was quenched with H₂O (10 mL) at 25° C. and extracted with EtOAc (10 mL×4). The combined organic layers were washed with brine (10 mL×2), dried over Na₂SO₄, filtered, and concentrated under reduced pressure to give a residue. The residue was purified by column chromatography (eluent of 0-10% MeOH in CH₂Cl₂) and then purified by prep-HPLC (column: Phenomenex C18 150*25 mm*10 um; mobile phase: [water (NH₄HCO₃)-ACN]; B %: 60%-90%, 8 min), 2-((2R,6R)-4-(4-(2-(2-aminopyridin-3-yl)-5-phenyl-3H-imidazo[4,5-b]pyridin-3-yl)benzyl)-2,6-dimethylpiperazin-1-yl)pyrimidine-4-carbonitrile (Example 196, 30 mg, yield: 12.3%) was obtained as a brown lyophilized powder. MS: m/z=593.3 [M+H]⁺. ¹H NMR (400 MHz, Dimethylsulfoxide-d₆) δ 8.72 (d, J=4.8 Hz, 1H), 8.27 (d, J=8.4 Hz, 1H), 8.05-7.97 (m, 4H), 7.54 (d, J=8.4 Hz, 2H), 7.49-7.44 (m, 4H), 7.42-7.38 (m, 1H), 7.25 (d, J=4.8 Hz, 1H), 7.17 (dd, J=7.6, 1.6 Hz, 1H), 7.05 (br s, 2H), 6.45-6.32 (m, 1H), 4.35-4.20 (m, 2H), 3.87 (d, J=14.0 Hz, 1H), 3.69 (d, J=14.0 Hz, 1H), 3.01-2.88 (m, 2H), 2.58-2.57 (m, 1H), 2.55-2.54 (m, 1H), 1.26 (d, J=6.4 Hz, 6H).

### Example 197: 6-((2R,6R)-4-(4-(2-(2-aminopyridin-3-yl)-5-phenyl-3H-imidazo[4,5-b]pyridin-3-yl)benzyl)-2,6-dimethylpiperazin-1-yl)pyrimidine-4-carbonitrile

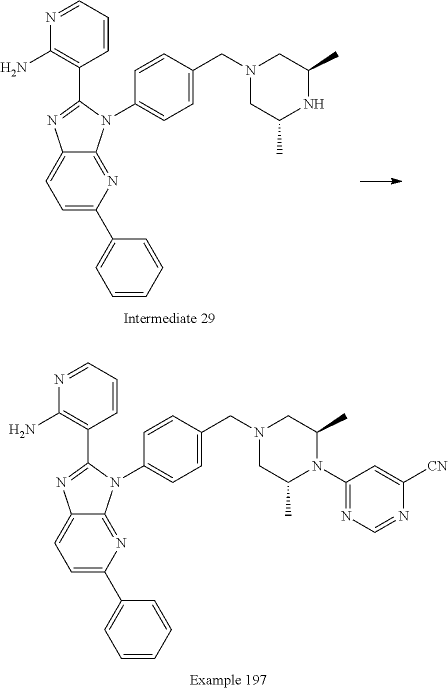 [SMILES: C[C@@H]1CN(Cc2ccc(-n3c(-c4cccnc4N)nc4ccc(-c5ccccc5)nc43)cc2)C[C@@H](C)N1 ; C[C@@H]1CN(Cc2ccc(-n3c(-c4cccnc4N)nc4ccc(-c5ccccc5)nc43)cc2)C[C@@H](C)N1c1cc(C#N)ncn1]

To a solution of Intermediate 29 (100 mg, 204 μmol) in NMP (2 mL) was added DIEA (106 mg, 817 μmol) and 6-chloropyrimidine-4-carbonitrile (31 mg, 225 μmol). The mixture was heated under microwave irradiation and stirred at 160° C. for 1 hr, the reaction mixture was filtered to get a liquid. The liquid was purified by prep-HPLC (column: Waters xbridge 150*25 mm 10 um; mobile phase: [water (NH₄HCO₃)-ACN]; B %: 52%-82%, 8 min), 6-((2R,6R)-4-(4-(2-(2-aminopyridin-3-yl)-5-phenyl-3H-imidazo[4,5-b]pyridin-3-yl)benzyl)-2,6-dimethylpiperazin-1-yl)pyrimidine-4-carbonitrile (Example 197, 31.3 mg, yield: 25%) was obtained as a gray lyophilized powder. MS: m/z=593.4 [M+H]⁺. ¹H NMR (400 MHz, Methanol-d₄) δ 8.58 (s, 1H), 8.19 (d, J=8.4 Hz, 1H), 8.03 (d, J=7.2 Hz, 2H), 7.98 (dd, J=4.8, 1.6 Hz, 1H), 7.94 (d, J=8.4 Hz, 1H), 7.57 (d, J=8.4 Hz, 2H), 7.47-7.40 (m, 5H), 7.33 (dd, J=8.0, 1.6 Hz, 1H), 7.23 (s, 1H), 6.48 (dd, J=7.6, 5.2 Hz, 1H), 4.34-4.24 (m, 2H), 3.96 (d, J=13.6 Hz, 1H), 3.84 (d, J=13.6 Hz, 1H), 3.24-3.16 (m, 2H), 2.80-2.72 (m, 2H), 1.36 (d, J=6.4 Hz, 6H).

### Example 198: 4-(7-(4-(2-(2-Aminopyridin-3-yl)-5-phenyl-3H-imidazo[4,5-b]pyridin-3-yl)benzyl)-4,7-diazaspiro[2.5]octan-4-yl)pyrimidine-2-carbonitrile

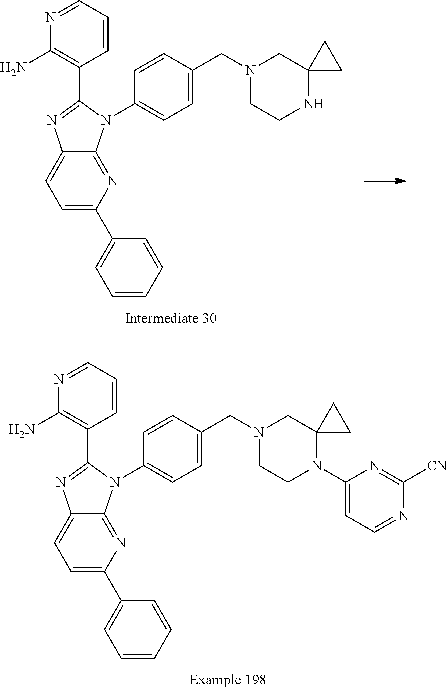 [SMILES: Nc1ncccc1-c1nc2ccc(-c3ccccc3)nc2n1-c1ccc(CN2CCNC3(CC3)C2)cc1 ; N#Cc1nccc(N2CCN(Cc3ccc(-n4c(-c5cccnc5N)nc5ccc(-c6ccccc6)nc54)cc3)CC23CC3)n1]

To a solution of Intermediate 30 (50 mg, 103 μmol) and 2-chloropyrimidine-4-carbonitrile (14.3 mg, 103 μmol) in NMP (1 mL) was added DIEA (66.3 mg, 513 μmol). The mixture was stirred at 130° C. for 0.5 hr under microwave. The reaction mixture was filtered. The residue was purified by prep-HPLC (column: Waters xbridge 150×25 mm 10 μm; mobile phase: [water (NH₄HCO₃)-ACN]; B %: 47%-77%, 10 min), 4-(7-(4-(2-(2-aminopyridin-3-yl)-5-phenyl-3H-imidazo[4,5-b]pyridin-3-yl)benzyl)-4,7-diazaspiro[2.5]octan-4-yl)pyrimidine-2-carbonitrile (Example 198, 15 mg, yield: 24%) was obtained as an off-white lyophilized powder. MS: m/z=591.4 [M+H]⁺. ¹H NMR (400 MHz, Dimethylsulfoxide-d₆) δ 8.35 (d, J=6.0 Hz, 1H), 8.27 (d, J=8.4 Hz, 1H), 8.07-7.93 (m, 4H), 7.52-7.34 (m, 7H), 7.23 (d, J=6.4 Hz, 1H), 7.12 (dd, J=7.6, 1.6 Hz, 1H), 7.04 (s, 2H), 6.29 (dd, J=7.6, 4.8 Hz, 1H), 3.59 (s, 2H), 2.51-2.50 (m, 6H), 1.25-0.75 (m, 4H).

### Example 199: 2-(7-(4-(2-(2-Aminopyridin-3-yl)-5-phenyl-3H-imidazo[4,5-b]pyridin-3-yl)benzyl)-4,7-diazaspiro[2.5]octan-4-yl)pyrimidine-4-carbonitrile

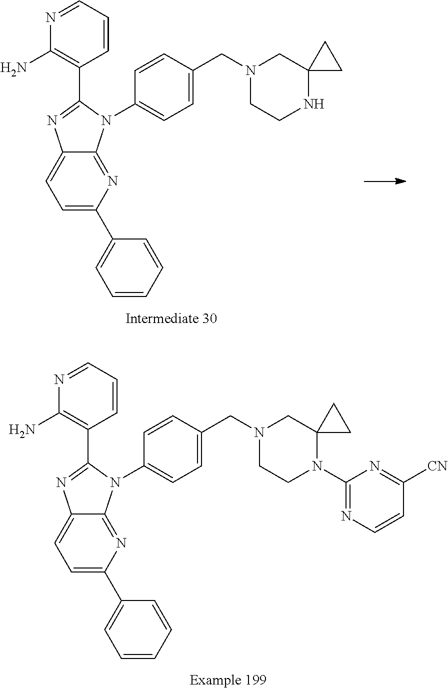 [SMILES: Nc1ncccc1-c1nc2ccc(-c3ccccc3)nc2n1-c1ccc(CN2CCNC3(CC3)C2)cc1 ; N#Cc1ccnc(N2CCN(Cc3ccc(-n4c(-c5cccnc5N)nc5ccc(-c6ccccc6)nc54)cc3)CC23CC3)n1]

To a solution of Intermediate 30 (100 mg, 205 μmol) and 2-chloropyrimidine-4-carbonitrile (28.6 mg, 205 μmol) in NMP (1 mL) was added DIEA (133 mg, 1.03 mmol). The mixture was stirred at 130° C. for 0.5 hr under microwave. The reaction mixture was filtered. The residue was purified by prep-HPLC (column: Waters xbridge 150×25 mm 10 μm; mobile phase: [water (NH₄HCO₃)-ACN]; B %: 59%-89%, 10 min), 2-(7-(4-(2-(2-aminopyridin-3-yl)-5-phenyl-3H-imidazo[4,5-b]pyridin-3-yl)benzyl)-4,7-diazaspiro[2.5]octan-4-yl)pyrimidine-4-carbonitrile (Example 199, 13.9 mg, yield: 10%) was obtained as a yellow lyophilized powder. MS: m/z=591.4 [M+H]⁺. ¹H NMR (400 MHz, Dimethylsulfoxide-d₆) δ 8.70 (d, J=4.8 Hz, 1H), 8.27 (d, J=8.4 Hz, 1H), 8.08-7.93 (m, 4H), 7.51-7.38 (m, 7H), 7.26 (d, J=4.8 Hz, 1H), 7.11 (dd, J=7.6, 1.6 Hz, 1H), 7.05 (s, 2H), 6.34-6.27 (dd, J=8.0, 4.8 Hz, 1H), 4.05-3.97 (m, 2H), 3.56 (s, 2H), 3.34-3.33 (m, 2H), 2.33 (s, 2H), 0.98-0.95 (m, 2H), 0.80-0.75 (m, 2H),

### Example 200: 6-(7-(4-(2-(2-Aminopyridin-3-yl)-5-phenyl-3H-imidazo[4,5-b]pyridin-3-yl)benzyl)-4,7-diazaspiro[2.5]octan-4-yl)pyrimidine-4-carbonitrile

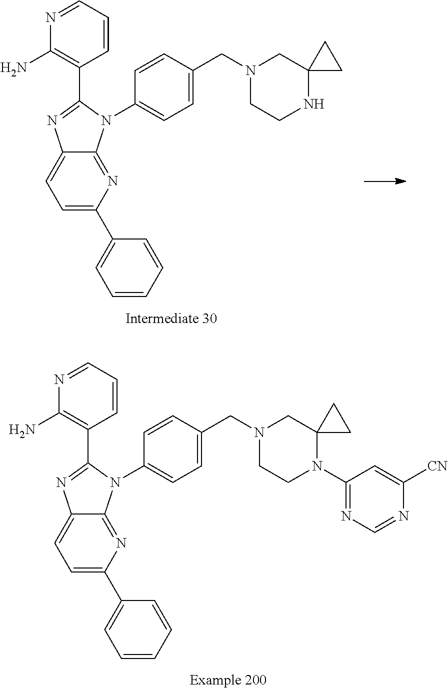 [SMILES: Nc1ncccc1-c1nc2ccc(-c3ccccc3)nc2n1-c1ccc(CN2CCNC3(CC3)C2)cc1 ; N#Cc1cc(N2CCN(Cc3ccc(-n4c(-c5cccnc5N)nc5ccc(-c6ccccc6)nc54)cc3)CC23CC3)ncn1]

To a solution of 3-(3-(4-((4,7-diazaspiro[2.5]octan-7-yl)methyl)phenyl)-5-phenyl-3H-imidazo[4,5-b]pyridin-2-yl)pyridin-2-amine (50 mg, 103 μmol) and 6-chloropyrimidine-4-carbonitrile (14.3 mg, 103 μmol) in NMP (1 mL) was added DLEA (66.3 mg, 513 μmol). The mixture was stirred at 130° C. for 0.5 hr under microwave. The reaction mixture was filtered. The residue was purified by prep-HPLC (column: Waters xbridge 150×25 mm 10 μm; mobile phase: [water (NH₄HCO₃)-ACN]; B %: 49%-79%, 10 min), 6-(7-(4-(2-(2-aminopyridin-3-yl)-5-phenyl-3H-imidazo[4,5-b]pyridin-3-yl)benzyl)-4,7-diazaspiro[2.5]octan-4-yl)pyrimidine-4-carbonitrile (Example 200, 18.9 mg, yield: 29%) was obtained as an off-white lyophilized powder. MS: m/z=591.4 [M+H]⁺. ¹H NMR (400 MHz, Dimethylsulfoxide-d₆) δ 8.66 (d, J=1.2 Hz, 1H), 8.10-7.86 (m, 4H), 7.54 (s, 1H), 7.49-7.39 (m, 7H), 7.12 (dd, J=7.2, 1.6 Hz, 1H), 7.07-6.96 (m, 2H), 6.29 (dd, J=7.6, 4.8 Hz, 1H), 3.59 (br s, 2H), 2.48-2.44 (m, 6H), 1.25-0.75 (m, 4H).

### Example 201: 4-((3-(4-(2-(2-Aminopyridin-3-yl)-5-phenyl-3H-imidazo[4,5-b]pyridin-3-yl)benzyl)-3-azabicyclo[3.2.1]octan-8-yl)amino)pyrimidine-2-carbonitrile

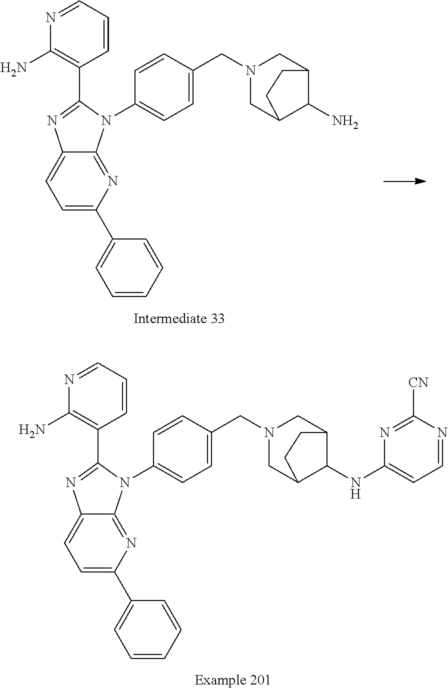 [SMILES: Nc1ncccc1-c1nc2ccc(-c3ccccc3)nc2n1-c1ccc(CN2CC3CCC(C2)C3N)cc1 ; N#Cc1nccc(NC2C3CCC2CN(Cc2ccc(-n4c(-c5cccnc5N)nc5ccc(-c6ccccc6)nc54)cc2)C3)n1]

Intermediate 33 (100 mg, 199 μmol), 4-chloropyrimidine-2-carbonitrile (27.8 mg, 199 μmol) and DIEA (129 mg, 997 μmol) were taken up into a microwave tube in NMP (2 mL). The sealed tube was heated at 160° C. for 1 hr under microwave. After purified by prep-HPLC (column: Welch Xtimate 150×25 mm×5 μm; mobile phase: [water (NH₄HCO₃)-ACN]; B %. 60%-90%, 20 min), 4-((3-(4-(2-(2-aminopyridin-3-yl)-5-phenyl-3H-imidazo[4,5-b]pyridin-3-yl)benzyl)-3-azabicyclo[3.2.1]octan-8-yl)amino)pyrimidine-2-carbonitrile (Example 201, 12 mg, yield: 9.3%) was obtained as a brown solid. MS: m/z=605.4 [M+H]⁺. ¹H NMR (400 MHz, Dimethylsulfoxide-d₆) δ 8.27 (d, J=8.4 Hz, 1H), 8.14 (d, J=6.4 Hz, 1H), 8.06-7.98 (m, 5H), 7.51-7.44 (m, 6H), 7.42-7.37 (m, 1H), 7.15 (dd, J=7.6, 1.2 Hz, 1H), 7.06 (br s, 2H), 6.98 (d, J=5.2 Hz, 1H), 6.36 (dd, J=7.6, 4.8 Hz, 1H), 3.87-3.82 (m, 1H), 3.62 (s, 2H), 2.54-2.52 (m, 4H), 2.28-2.22 (m, 2H), 1.81-1.73 (m, 4H).

### Example 202: 6-((3-(4-(2-(2-Aminopyridin-3-yl)-5-phenyl-3H-imidazo[4,5-b]pyridin-3-yl)benzyl)-3-azabicyclo[3.2.1]octan-8-yl)amino)pyrimidine-4-carbonitrile

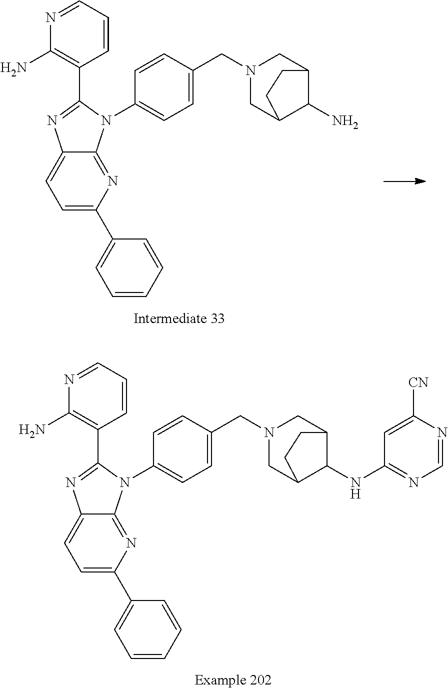 [SMILES: Nc1ncccc1-c1nc2ccc(-c3ccccc3)nc2n1-c1ccc(CN2CC3CCC(C2)C3N)cc1 ; N#Cc1cc(NC2C3CCC2CN(Cc2ccc(-n4c(-c5cccnc5N)nc5ccc(-c6ccccc6)nc54)cc2)C3)ncn1]

Intermediate 33 (100 mg, 199 μmol), 4-chloropyrimidine-2-carbonitrile (27.8 mg, 199 μmol) and DIEA (129 mg, 997 μmol) were taken up into a microwave tube in NMP (2 mL). The sealed tube was heated at 160° C. for 1 hr under microwave. After purified by prep-HPLC (column: Welch Xtimate 150×25 mm×5 μm; mobile phase: [water (NH₄HCO₃)-ACN]; B %: 60%-90%, 20 min), 6-((3-(4-(2-(2-aminopyridin-3-yl)-5-phenyl-3H-imidazo[4,5-b]pyridin-3-yl)benzyl)-3-azabicyclo[3.2.1]octan-8-yl)amino)pyrimidine-4-carbonitrile (Example 202, 14.4 mg, yield: 11%) was obtained as a brown solid. MS: m/z=605.3 [M+H]⁺. ¹H NMR (400 MHz, Dimethylsulfoxide-d₆) δ 8.57-8.46 (m, 1H), 8.27 (d, J=8.4 Hz, 1H), 8.08 (d, J=5.6 Hz, 1H), 8.04-7.97 (m, 4H), 7.50-7.43 (m, 6H), 7.41-7.37 (m, 1H), 7.22 (s, 1H), 7.15 (dd, J=7.6, 1.6 Hz, 1H), 7.05 (s, 2H), 6.35 (dd, J=7.6, 4.8 Hz, 1H), 3.94-3.88 (m, 1H), 3.61 (s, 2H), 2.53-2.51 (m, 4H), 2.27-2.22 (m, 2H), 1.79-1.69 (m, 4H).

### Example 203: 4-(3-(4-(2-(2-Aminopyridin-3-yl)-5-phenyl-3H-imidazo[4,5-b]pyridin-3-yl)benzyl)-3,8-diazabicyclo[3.2.1]octan-8-yl)pyrimidine-2-carbonitrile

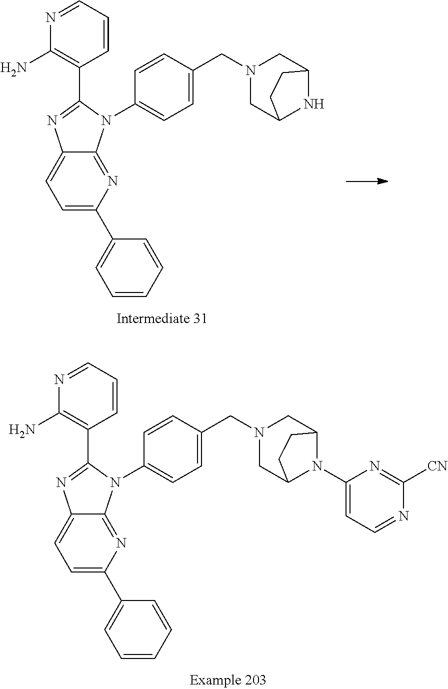 [SMILES: Nc1ncccc1-c1nc2ccc(-c3ccccc3)nc2n1-c1ccc(CN2CC3CCC(C2)N3)cc1 ; N#Cc1nccc(N2C3CCC2CN(Cc2ccc(-n4c(-c5cccnc5N)nc5ccc(-c6ccccc6)nc54)cc2)C3)n1]

A solution of Intermediate 31 (80 mg, 164 μmol), 4-chloropyrimidine-2-carbonitrile (25.2 mg, 180 μmol) and DIEA (106 mg, 820 μmol) were taken up into a microwave tube in NMP (2 mL). The sealed tube was heated at 130° C. for 1 hr under microwave. The reaction mixture was filtered, the filtrate was concentrated under reduced pressure to give a residue. The residue was purified by prep-HPLC (column: Waters xbridge 150*25 mm 10 μm; mobile phase: [water (NH₄HCO₃)-ACN]; B %: 55%-85%, 10 min), 4-(3-(4-(2-(2-aminopyridin-3-yl)-5-phenyl-3H-imidazo[4,5-b]pyridin-3-yl)benzyl)-3,8-diazabicyclo[3.2.1]octan-8-yl)pyrimidine-2-carbonitrile (Example 203, 10.5 mg, yield: 11%) was obtained as a light-yellow solid. MS: m/z=591.4 [M+H]⁺. ¹H NMR (400 MHz, Dimethylsulfoxide-d₆) δ 8.27 (d, J=8.4 Hz, H), 8.24 (d, J=6.4 Hz, H), 8.04-7.97 (m, 4H), 7.50-7.44 (m, 6H), 7.42-7.38 (m, 1H), 7.11 (dd, J=7.6, 1.6 Hz, 1H), 7.05 (br s, 2H), 7.02 (d, J=6.4 Hz, 1H), 6.31 (dd, J=7.6, 4.8 Hz, 1H), 4.84-4.76 (m, 1H), 4.57-4.51 (m, 1H), 3.59 (s, 2H), 2.75-2.68 (m, 2H), 2.31-2.26 (m, 2H), 2.06-1.82 (m, 4H).

### Example 204: 2-(3-(4-(2-(2-Aminopyridin-3-yl)-5-phenyl-3H-imidazo[4,5-b]pyridin-3-yl)benzyl)-3,8-diazabicyclo[3.2.1]octan-8-yl)pyrimidine-4-carbonitrile

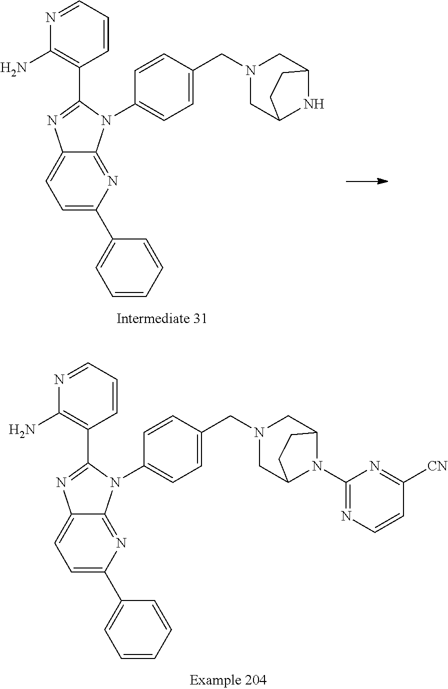 [SMILES: Nc1ncccc1-c1nc2ccc(-c3ccccc3)nc2n1-c1ccc(CN2CC3CCC(C2)N3)cc1 ; N#Cc1ccnc(N2C3CCC2CN(Cc2ccc(-n4c(-c5cccnc5N)nc5ccc(-c6ccccc6)nc54)cc2)C3)n1]

Following the general procedure of Example 203 the reaction of Intermediate 31 (80 mg, 164 μmol) with 2-chloropyrimidine-4-carbonitrile (25.2 mg, 180 μmol) was carried out. After purified by prep-HPLC (column: Waters xbridge 150*25 mm 10 um; mobile phase: [water (NH₄HCO₃)-ACN]; B %: 63%-93%, 10 min), 2-(3-(4-(2-(2-aminopyridin-3-yl)-5-phenyl-3H-imidazo[4,5-b]pyridin-3-yl)benzyl)-3,8-diazabicyclo[3.2.1]octan-8-yl)pyrimidine-4-carbonitrile (Example 204, 6.7 mg, yield: 6.9%) was obtained as a light-yellow powder. MS: m/z=591.5 [M+H]⁺. ¹H NMR (400 MHz, Dimethylsulfoxide-d₆) δ 8.64 (d, J=4.8 Hz, 1H), 8.26 (d, J=8.4 Hz, 1H), 8.05-7.97 (m, 4H), 7.50-7.43 (m, 6H), 7.41-7.37 (m, 1H), 7.15-7.11 (m, 2H), 7.05 (br s, 2H), 6.33 (dd, J=7.6, 4.8 Hz, 1H), 4.72-4.64 (m, 2H), 3.58 (s, 2H), 2.72 (d, J=9.2 Hz, 2H), 2.30 (d, J=10.4 Hz, 2H), 2.04-1.96 (m, 2H), 1.90-1.85 (m, 2H).

### Example 205: 6-(3-(4-(2-(2-Aminopyridin-3-yl)-5-phenyl-3H-imidazo[4,5-b]pyridin-3-yl)benzyl)-3,8-diazabicyclo[3.2.1]octan-8-yl)pyrimidine-4-carbonitrile

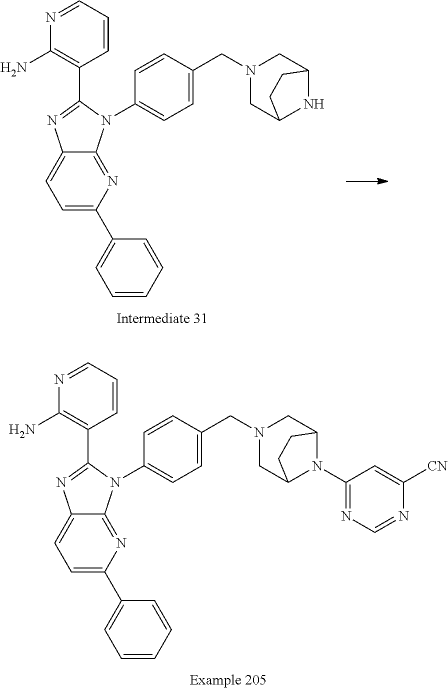 [SMILES: Nc1ncccc1-c1nc2ccc(-c3ccccc3)nc2n1-c1ccc(CN2CC3CCC(C2)N3)cc1 ; N#Cc1cc(N2C3CCC2CN(Cc2ccc(-n4c(-c5cccnc5N)nc5ccc(-c6ccccc6)nc54)cc2)C3)ncn1]

Following the general procedure of Example 203, the reaction of Intermediate 31 (80 mg, 164 μmol) with 6-chloropyrimidine-4-carbonitrile (25.2 mg, 180 μmol) was carried out. After purified by prep-HPLC (column: Waters xbridge 150*25 mm 10 um; mobile phase: [water (NH₄HCO₃)-ACN]; B %: 55%-85%, 10 min), 6-(3-(4-(2-(2-aminopyridin-3-yl)-5-phenyl-3H-imidazo[4,5-b]pyridin-3-yl)benzyl)-3,8-diazabicyclo[3.2.1]octan-8-yl)pyrimidine-4-carbonitrile (Example 205, 11 mg, yield: 11.4%) was obtained as a light-yellow powder. MS: m/z=591.5 [M+H]⁺. ¹H NMR (400 MHz, Dimethylsulfoxide-d₆) δ 8.54 (s, 1H), 8.27 (d, J=8.4 Hz, 1H), 8.05-7.97 (m, 4H), 7.50-7.43 (m, 7H), 7.41-7.37 (m, 1H), 7.13 (dd, J=7.6, 1.6 Hz, 1H), 7.05 (br s, 2H), 6.30 (dd, J=7.6, 4.4 Hz, 1H), 4.90-4.84 (m, 1H), 4.59-4.53 (m, 1H), 3.59 (s, 2H), 2.72 (d, J=11.2 Hz, 2H), 2.30 (d, J=10.4 Hz, 2H), 2.05-2.00 (m, 2H), 1.90-1.85 (m, 2H).

### Example 206: (S)-4-(4-(4-(2-(2-Aminopyridin-3-yl)-5-phenyl-3H-imidazo[4,5-b]pyridin-3-yl)benzyl)-2-methylpiperazin-1-yl)pyrimidine-2-carbonitrile

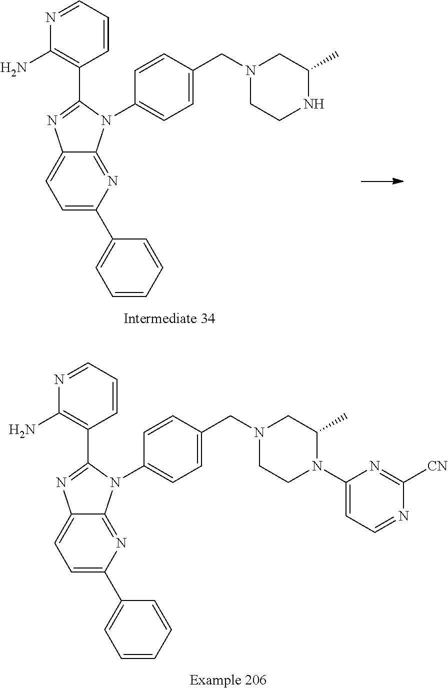 [SMILES: C[C@H]1CN(Cc2ccc(-n3c(-c4cccnc4N)nc4ccc(-c5ccccc5)nc43)cc2)CCN1 ; C[C@H]1CN(Cc2ccc(-n3c(-c4cccnc4N)nc4ccc(-c5ccccc5)nc43)cc2)CCN1c1ccnc(C#N)n1]

To a solution of Intermediate 34 (50 mg, 105 μmol) and 2-chloropyrimidine-4-carbonitrile (14.7 mg, 105 μmol) in NMP (1 mL) was added DIEA (67.9 mg, 526 μmol). The mixture was stirred at 130° C. for 0.5 hr under microwave. The reaction mixture was filtered. The reaction mixture was purified by prep-HPLC (column: Waters xbridge 150×25 mm 10 μm; mobile phase: [water (NH₄HCO₃)-ACN]; B %: 53%-83%, 10 min), (S)-4-(4-(4-(2-(2-aminopyridin-3-yl)-5-phenyl-3H-imidazo[4,5-b]pyridin-3-yl)benzyl)-2-methylpiperazin-1-yl)pyrimidine-2-carbonitrile (Example 206, 28 mg, yield: 44%) was obtained as an off-white lyophilized powder. MS: m/z=579.4 [M+H]⁺. ¹H NMR (400 MHz, Dimethylsulfoxide-d₆) δ 8.31-8.25 (m, 2H), 8.07-7.97 (m, 4H), 7.55-7.53 (m, 2H), 7.50-7.45 (m, 4H), 7.42-7.37 (m, 1H), 7.17 (dd, J=7.6, 1.6 Hz, 1H), 7.10-7.01 (m, 3H), 6.39 (dd, J=7.6, 4.8 Hz, 1H), 4.56 (br s, 1H), 4.22 (br s, 1H), 3.71 (d, J=14.0 Hz, 1H), 3.60-3.52 (d, J=14.0 Hz, 1H), 3.23-3.16 (m, 1H), 2.98 (br d, J=11.2 Hz, 1H), 2.76 (d, J=14.0 Hz 1H), 2.21 (dd, J=11.2, 3.2 Hz, 1H), 2.16-2.02 (m, 1H), 1.24 (d, J=6.8 Hz, 3H).

### Example 207: (S)-2-(4-(4-(2-(2-Aminopyridin-3-yl)-5-phenyl-3H-imidazo[4,5-b]pyridin-3-yl)benzyl)-2-methylpiperazin-1-yl)pyrimidine-4-carbonitrile

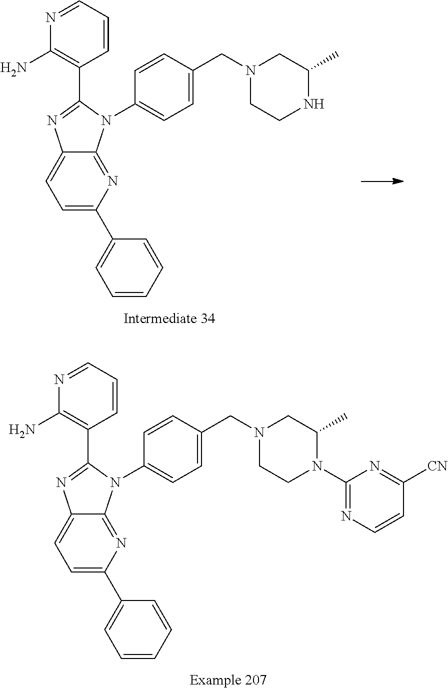 [SMILES: C[C@H]1CN(Cc2ccc(-n3c(-c4cccnc4N)nc4ccc(-c5ccccc5)nc43)cc2)CCN1 ; C[C@H]1CN(Cc2ccc(-n3c(-c4cccnc4N)nc4ccc(-c5ccccc5)nc43)cc2)CCN1c1nccc(C#N)n1]

To a solution of Intermediate 207 (100 mg, 210 μmol) and 2-chloropyrimidine-4-carbonitrile (29.3 mg, 210 μmol) in NMP (2 mL) was added DIEA (136 mg, 1.05 mmol). The mixture was stirred at 130° C. for 0.5 hr under microwave. The reaction mixture was filtered. The reaction mixture was purified by prep-HPLC (column: Waters xbridge 150×25 mm 10 μm; mobile phase: [water (NH₄HCO₃)-ACN]; B %: 59%-89%, 10 min), (S)-2-(4-(4-(2-(2-aminopyridin-3-yl)-5-phenyl-3H-imidazo[4,5-b]pyridin-3-yl)benzyl)-2-methylpiperazin-1-yl)pyrimidine-4-carbonitrile (Example 207, 28.5 mg, yield: 23%) was obtained as a yellow lyophilized powder. MS: m/z=579.4 [M+H]⁺. ¹H NMR (400 MHz, Dimethylsulfoxide-d₆) δ 8.65 (d, J=4.4 Hz, 1H), 8.27 (d, J=8.4 Hz, 1H), 8.08-7.96 (m, 4H), 7.56-7.53 (m, 2H), 7.49-7.44 (m, 4H), 7.42-7.37 (m, 1H), 7.20-7.12 (m, 2H), 7.05 (s, 2H), 6.39 (dd, J=7.6, 4.8 Hz, 1H), 4.76 (br s, 1H), 4.39 (d, J=12.0 Hz, 1H), 3.70 (d, J=13.6 Hz, 1H), 3.54 (d, J=14.0 Hz, 1H), 3.20 (dt, J=13.2, 3.2 Hz, 1H), 2.96 (d, J=11.2 Hz, 1H), 2.74 (d, J=11.2 Hz, 1H), 2.20 (dd, J=11.2, 4.0 Hz, 1H), 2.14-2.09 (m, 1H), 1.23 (d, J=6.4 Hz, 3H).

### Example 208: (S)-6-(4-(4-(2-(2-Aminopyridin-3-yl)-5-phenyl-3H-imidazo[4,5-b]pyridin-3-yl)benzyl)-2-methylpiperazin-1-yl)pyrimidine-4-carbonitrile

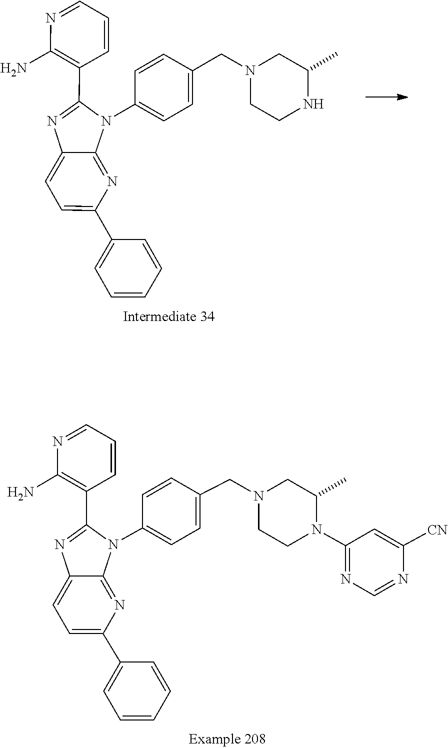 [SMILES: C[C@H]1CN(Cc2ccc(-n3c(-c4cccnc4N)nc4ccc(-c5ccccc5)nc43)cc2)CCN1 ; C[C@H]1CN(Cc2ccc(-n3c(-c4cccnc4N)nc4ccc(-c5ccccc5)nc43)cc2)CCN1c1cc(C#N)ncn1]

To a solution of Intermediate 34 (50 mg, 105 μmol) and 6-chloropyrimidine-4-carbonitrile (14.7 mg, 105 μmol) in NMP (I mL) was added DIEA (67.9 mg, 526 μmol). The mixture was stirred at 130° C. for 0.5 hr under microwave. The reaction mixture was filtered. The residue was purified by prep-HPLC (column: Waters xbridge 150×25 mm 10 μm; mobile phase: [water (NH₄HCO₃)-ACN]; B %: 53%-83%, 10 min), (S)-6-(4-(4-(2-(2-aminopyridin-3-yl)-5-phenyl-3H-imidazo[4,5-b]pyridin-3-yl)benzyl)-2-methylpiperazin-1-yl)pyrimidine-4-carbonitrile (Example 208, 25.2 mg, 40.6% yield) was obtained as an off-white lyophilized powder. MS: m/z=579.4 [M+H]⁺. ¹H NMR (400 MHz, Dimethylsulfoxide-d₆) δ 8.58 (s, 1H), 8.27 (d, J=8.4 Hz, 1H), 8.08-7.95 (m, 4H), 7.56-7.44 (m, 7H), 7.41-7.37 (m, 1H), 7.16 (dd, J=8.0, 2.0 Hz, 1H), 7.04 (s, 2H), 6.38 (dd, J=7.6, 4.8 Hz, 1H), 4.63-4.29 (m, 2H), 3.70 (d, J=13.2 Hz, 1H), 3.55 (d, J=13.2 Hz, 1H), 3.22-3.19 (m, 1H), 2.97 (d, J=11.2 Hz, 1H), 2.73 (d, J=11.2 Hz, 1H), 2.20 (dd, J=11.2, 3.2 Hz, 1H), 2.13-2.07 (m, 1H), 1.24 (d, J=6.8 Hz, 3H).

### Example 209: 4-((4-(2-(2-Aminopyridin-3-yl)-5-phenyl-3H-imidazo[4,5-b]pyridin-3-yl)benzyl)amino)-1,3,5-triazine-2-carbonitrile

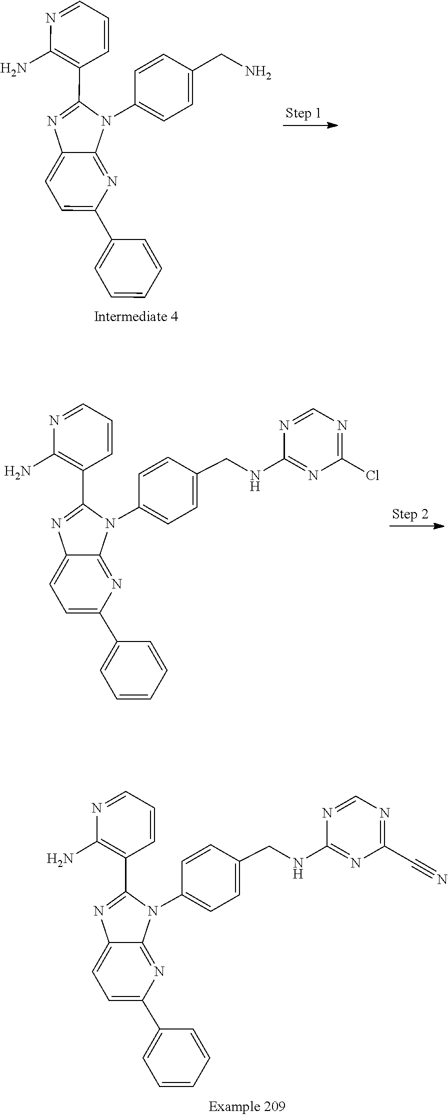 [SMILES: NCc1ccc(-n2c(-c3cccnc3N)nc3ccc(-c4ccccc4)nc32)cc1 ; Nc1ncccc1-c1nc2ccc(-c3ccccc3)nc2n1-c1ccc(CNc2ncnc(Cl)n2)cc1 ; N#Cc1ncnc(NCc2ccc(-n3c(-c4cccnc4N)nc4ccc(-c5ccccc5)nc43)cc2)n1]

### Step 1: N-[[4-[2-(2-amino-3-pyridyl)-5-phenyl-imidazo[4,5-b]pyridin-3-yl]phenyl]methyl]-4-chloro-1,3,5-triazin-2-amine

To a solution of Intermediate 4 (140 mg, 357 μmol) in THF (4 mL) and H₂O (1 mL) was added K₂CO₃ (148 mg, 1.07 mmol) and stirred at 0° C. for 15 min. Then 2,4-dichloro-1,3,5-triazine (70 mg, 464 μmol) in THF (1 mL) was added into the mixture and stirred at 0° C. for 1.5 hr. The reaction was filtered to give the filter liquor and concentrated under reduced pressure to give a residue. The residue was purified by prep-TLC (MeOH:CH₂Cl₂=1:10) to give N-[[4-[2-(2-amino-3-pyridyl)-5-phenyl-imidazo[4,5-b]pyridin-3-yl]phenyl]methyl]-4-chloro-1,3,5-triazin-2-amine (6.8 mg, yield: 3.4%) as a light-yellow solid. MS: m/z=506.3 [M+H]⁺. ¹H NMR (400 MHz, Dimethylsulfoxide-d₆) δ 9.28-9.19 (m, 1H), 8.51-8.44 (m, 1H), 8.26 (d, J=8.4 Hz, 1H), 8.04-7.97 (m, 4H), 7.49-7.43 (m, 5H), 7.44-7.36 (m, 2H), 7.21 (d, J=7.2 Hz, 1H), 6.95 (d, J=7.2 Hz, 1H), 6.44-6.37 (m, 1H), 4.71-4.59 (m, 2H).

### Step 2: 4-((4-(2-(2-aminopyridin-3-yl)-5-phenyl-3H-imidazo[4,5-b]pyridin-3-yl)benzyl)amino)-1,3,5-triazine-2-carbonitrile

To a solution of N-(4-(2-(2-aminopyridin-3-yl)-5-phenyl-3H-imidazo[4,5-b]pyridin-3-yl)benzyl)-4-chloro-1,3,5-triazin-2-amine (110 mg, 217 μmol) and DABCO (4.88 mg, 43.5 μmol) in DMSO (1 mL) was added KCN (28.3 mg, 435 μmol) and stirred at 0° C. for 15 min. Then the mixture was stirred at 60° C. for 16 hr. The mixture was quenched with 15 mL H₂O and extracted with CH₂Cl₂ (15 mL×3), the combined organic phase was dried over anhydrous Na₂SO₄, filtered, and concentrated to give a residue. The residue was purified by prep-HPLC (column: Waters xbridge 150×25 mm 10 μm; mobile phase: [water (NH₄HCO₃)-ACN]; B %: 37%-67%, 9 min), 4-((4-(2-(2-aminopyridin-3-yl)-5-phenyl-3H-imidazo[4,5-b]pyridin-3-yl)benzyl)amino)-1,3,5-triazine-2-carbonitrile (Example 209, 7.9 mg, yield: 7.3%) was obtained as a yellow solid. MS: m/z=497.2 [M+H]⁺. ¹H NMR (400 MHz, Dimethylsulfoxide-d₆) δ9.44 (t, J=6.4 Hz, 1H), 8.71 (d, J=22.0 Hz, 1H), 8.26 (d, J=8.4 Hz, 1H), 8.04-7.96 (m, 4H), 7.50-7.44 (m, 6H), 7.42-7.37 (m, 1H), 7.22-7.19 (m, 1H), 6.93 (d, J=9.2 Hz, 2H), 6.41 (dd, J=7.6, 4.8 Hz, 1H), 4.72-4.64 (m, 1H).

### Example 210: 4-((4-(2-(2-aminopyridin-3-yl)-5-phenyl-3H-imidazo[4,5-b]pyridin-3-yl)benzyl)amino)-6-hydroxy-1,3,5-triazine-2-carbonitrile

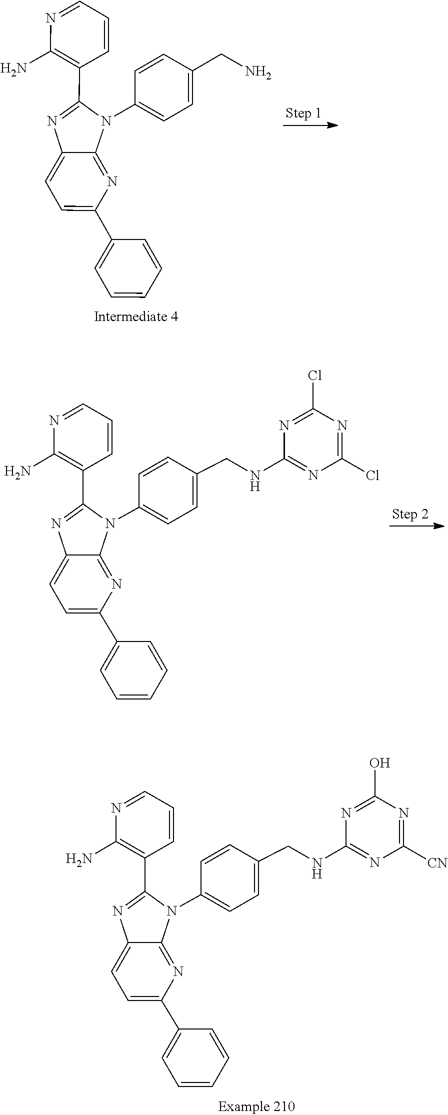 [SMILES: NCc1ccc(-n2c(-c3cccnc3N)nc3ccc(-c4ccccc4)nc32)cc1 ; Nc1ncccc1-c1nc2ccc(-c3ccccc3)nc2n1-c1ccc(CNc2nc(Cl)nc(Cl)n2)cc1 ; N#Cc1nc(O)nc(NCc2ccc(-n3c(-c4cccnc4N)nc4ccc(-c5ccccc5)nc43)cc2)n1]

### Step 1: N-(4-(2-(2-Aminopyridin-3-yl)-5-phenyl-3H-imidazo[4,5-b]pyridin-3-yl)benzyl)-4,6-dichloro-1,3,5-triazin-2-amine

Following the general procedure of Example 209 step 1, the reaction of Intermediate 4 (200 mg, 404 μmol) with 2,4,6-trichloro-1,3,5-triazine (103 mg, 561 μmol) was carried out. After silica gel flash chromatography (Eluent of 50˜75% EtOAc in petroleum ether), N-(4-(2-(2-aminopyridin-3-yl)-5-phenyl-3H-imidazo[4,5-b]pyridin-3-yl)benzyl)-4,6-dichloro-1,3,5-triazin-2-amine (120 mg, yield: 42%) was obtained as a yellow solid. MS: m/z=540.2 [M+H]⁺. ¹H NMR (400 MHz, Dimethylsulfoxide-d₆) δ 9.66 (t, J=6.4 Hz, 1H), 8.27 (d, J=8.4 Hz, 1H), 8.05-7.96 (m, 4H), 7.51-7.47 (m, 4H), 7.47-7.43 (m, 2H), 7.42-7.38 (m, 1H), 7.21 (dd, J=7.6, 2.0 Hz, 1H), 6.95 (br s, 2H), 6.41 (dd, J=7.6, 4.8 Hz, 1H), 4.65 (d, J=6.4 Hz, 2H).

### Step 2: 4-((4-(2-(2-aminopyridin-3-yl)-5-phenyl-3H-imidazo[4,5-b]pyridin-3-yl)benzyl)amino)-6-hydroxy-1,3,5-triazine-2-carbonitrile

To a solution of N-(4-(2-(2-aminopyridin-3-yl)-5-phenyl-3H-imidazo[4,5-b]pyridin-3-yl)benzyl)-4,6-dichloro-1,3,5-triazin-2-amine (130 mg, 241 μmol) in DMSO (3 mL) was added DABCO (5.4 mg, 48.1 μmol) and KCN (50 mg, 768 μmol), the mixture was stirred at 60° C. for 16 hr. The reaction mixture was diluted with H₂O (5 mL) and extracted with CH₂Cl₂ (10 mL×3). The combined organic layers were washed with 10 mL brine, dried over Na₂SO₄, filtered, and concentrated under reduced pressure to give a residue. After purified by prep-HPLC (column: Welch Xtimate C18 150×25 mm 5 μm; mobile phase: [water (NH₄HCO₃)-ACN]; B %: 22%-52%, 12 min), 4-((4-(2-(2-aminopyridin-3-yl)-5-phenyl-3H-imidazo[4,5-b]pyridin-3-yl)benzyl)amino)-6-hydroxy-1,3,5-triazine-2-carbonitrile (Example 210, 5.2 mg, yield: 4.2%) was obtained as a light-yellow lyophilized powder. MS: m/z=513.3 [M+H]⁺. ¹H NMR (400 MHz, Dimethylsulfoxide-d₆) δ 12.16 (br s, 1H), 8.39 (br s, 1H), 8.27 (d, J=8.4 Hz, 1H), 8.04-7.97 (m, 4H), 7.50-7.44 (m, 6H), 7.42-7.38 (m, 1H), 7.22 (d, J=7.6 Hz, 1H), 6.94 (br s, 2H), 6.42 (dd, J=7.6, 4.8 Hz, 1H), 4.67-4.58 (m, 2H).

### Example 211: 4-(8-(4-(2-(2-Aminopyridin-3-yl)-5-phenyl-3H-imidazo[4,5-b]pyridin-3-yl)benzyl)-3,8-diazabicyclo[3.2.1]octan-3-yl)pyrimidine-2-carbonitrile

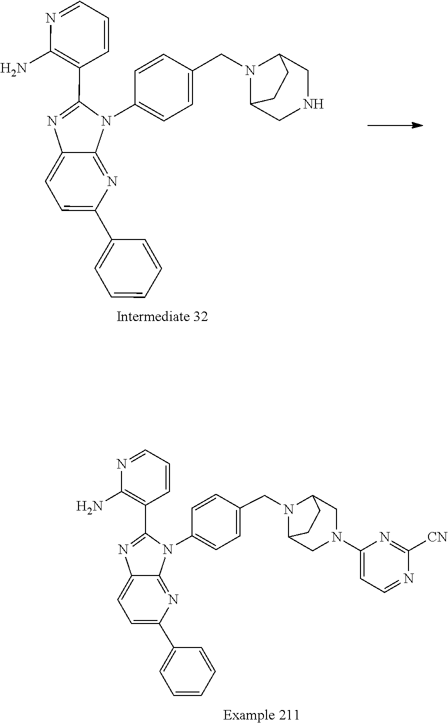 [SMILES: Nc1ncccc1-c1nc2ccc(-c3ccccc3)nc2n1-c1ccc(CN2C3CCC2CNC3)cc1 ; N#Cc1nccc(N2CC3CCC(C2)N3Cc2ccc(-n3c(-c4cccnc4N)nc4ccc(-c5ccccc5)nc43)cc2)n1]

Following the general procedure of Example 203, the reaction of Intermediate 32 (80 mg, 164 μmol) with 4-chloropyrimidine-2-carbonitrile (25.2 mg, 180 μmol) was carried out. After purified by prep-HPLC (column: Waters xbridge 150*25 mm 10 μm; mobile phase: [water (NH₄HCO₃)-ACN]; B %: 50%-80%, 10 min), 4-(8-(4-(2-(2-aminopyridin-3-yl)-5-phenyl-3H-imidazo[4,5-b]pyridin-3-yl)benzyl)-3,8-diazabicyclo[3.2.1]octan-3-yl)pyrimidine-2-carbonitrile (Example 211, 8.4 mg, yield; 8.7%) was obtained as a light-yellow powder. MS: m/z=591.4 [M+H]⁺. ¹H NMR (400 MHz, Dimethylsulfoxide-d₆) δ 8.27 (d, J=5.6 Hz, 1H), 8.26 (d, J=3.6 Hz, 1H), 8.05-7.97 (m, 4H), 7.59 (d, J=8.4 Hz, 2H), 7.50-7.43 (m, 4H), 7.41-7.37 (m, 1H), 7.18 (dd, J=7.6, 2.0 Hz, 1H), 7.03-7.00 (m, 3H), 6.41 (dd, J=7.6, 4.8 Hz, 1H), 4.28-4.20 (m, 1H), 3.75-3.68 (m, 3H), 3.40-3.37 (m, 2H), 3.24-3.06 (m, 2H), 2.05-2.00 (m, 2H), 1.57-1.50 (m, 2H).

### Example 212: 2-(8-(4-(2-(2-Aminopyridin-3-yl)-5-phenyl-3H-imidazo[4,5-b]pyridin-3-yl)benzyl)-3,8-diazabicyclo[3.2.1]octan-3-yl)pyrimidine-4-carbonitrile

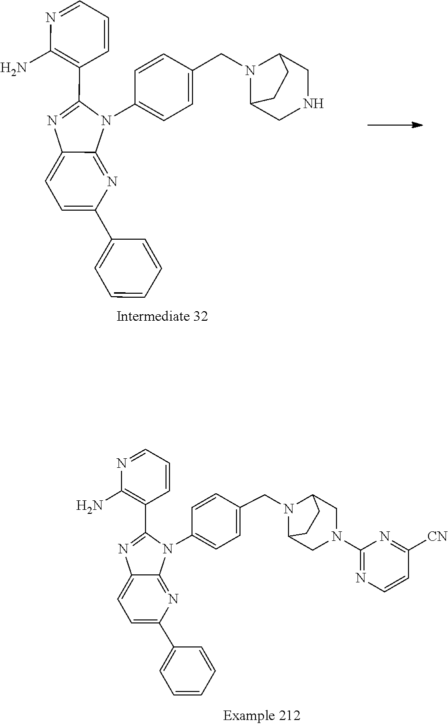 [SMILES: Nc1ncccc1-c1nc2ccc(-c3ccccc3)nc2n1-c1ccc(CN2C3CCC2CNC3)cc1 ; N#Cc1ccnc(N2CC3CCC(C2)N3Cc2ccc(-n3c(-c4cccnc4N)nc4ccc(-c5ccccc5)nc43)cc2)n1]

Following the general procedure of Example 203, the reaction of Intermediate 32 (80 mg, 164 μmol) with 2-chloropyrimidine-4-carbonitrile (25.2 mg, 180 μmol) was carried out. After purified by prep-HPLC (column; Waters xbridge 150*25 mm 10 μm, mobile phase: [water (NH₄HCO₃)-ACN]; B %: 57%-87%, 10 min), 2-(8-(4-(2-(2-aminopyridin-3-yl)-5-phenyl-3H-imidazo[4,5-b]pyridin-3-yl)benzyl)-3,8-diazabicyclo[3.2.1]octan-3-yl)pyrimidine-4-carbonitrile (Example 212, 7.3 mg, yield: 7.7%) as a light yellow-powder. MS: m/z=591.3 [M+H]⁺. ¹H NMR (400 MHz, Dimethylsulfoxide-d₆) δ 8.63 (d, J=4.4 Hz, 1H), 8.27 (d, J=8.4 Hz, 1H), 8.05-7.97 (m, 4H), 7.59 (d, J=8.4 Hz, 2H), 7.50-7.45 (m, 4H), 7.42-7.37 (m, 1H), 7.18 (dd, J=6.0, 1.6 Hz, 1H), 7.16 (d, J=4.4 Hz, 1H), 7.01 (br s, 2H), 6.41 (dd, J=7.6, 4.8 Hz, 1H), 4.25-4.12 (m, 2H), 3.68 (s, 2H), 3.37-3.35 (m, 2H), 3.18-3.16 (m, 2H), 2.03-1.98 (m, 2H), 1.56-1.48 (m, 2H).

### Example 213: 6-(8-(4-(2-(2-Aminopyridin-3-yl)-5-phenyl-3H-imidazo[4,5-b]pyridin-3-yl)benzyl)-3,8-diazabicyclo[3.2.1]octan-3-yl)pyrimidine-4-carbonitrile

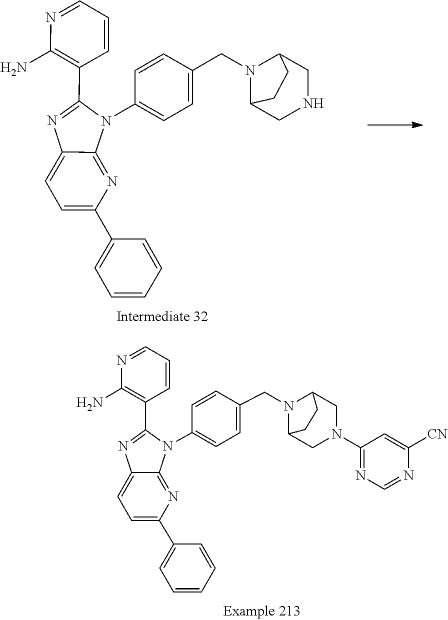 [SMILES: Nc1ncccc1-c1nc2ccc(-c3ccccc3)nc2n1-c1ccc(CN2C3CCC2CNC3)cc1 ; N#Cc1cc(N2CC3CCC(C2)N3Cc2ccc(-n3c(-c4cccnc4N)nc4ccc(-c5ccccc5)nc43)cc2)ncn1]

Following the general procedure of Example 203, the reaction of Intermediate 32 (80 mg, 164 μmol) with 6-chloropyrimidine-4-carbonitrile (25.2 mg, 180 μmol) was carried out. After purified by prep-HPLC (column: Waters xbridge 150*25 mm 10 μm; mobile phase: [water (NH₄HCO₃)-ACN]; B %: 50%-80%, 10 min), 6-(8-(4-(2-(2-aminopyridin-3-yl)-5-phenyl-3H-imidazo[4,5-b]pyridin-3-yl)benzyl)-3,8-diazabicyclo[3.2.1]octan-3-yl)pyrimidine-4-carbonitrile (Example 213, 13.2 mg, yield: 13.6%) was obtained as a light-yellow powder. MS: m/z=591.3 [M+H]⁺. ¹H NMR (400 MHz, Dimethylsulfoxide-d₆) δ 8.56 (s, 1H), 8.27 (d, J=8.4 Hz, 1H), 8.05-7.97 (m, 4H), 7.58 (d, J=8.4 Hz, 2H), 7.50-7.45 (m, 4H), 7.42-7.37 (m, 1H), 7.17 (dd, J=8.0, 1.6 Hz, 1H), 7.01 (br s, 2H), 6.41 (dd, J=7.6, 4.8 Hz, 1H), 4.45-4.30 (m, 1H), 3.78-3.68 (m, 3H), 3.45-3.37 (m, 2H), 3.26-3.18 (m, 1H), 3.14-3.03 (m, 1H), 2.05-2.00 (m, 2H), 1.57-1.46 (m, 2H).

### Example 214: 4-(4-(4-(2-(2-Aminopyridin-3-yl)-5-phenyl-3H-imidazo[4,5-b]pyridin-3-yl)benzyl)-1,4-diazepan-1-yl)pyrimidine-2-carbonitrile

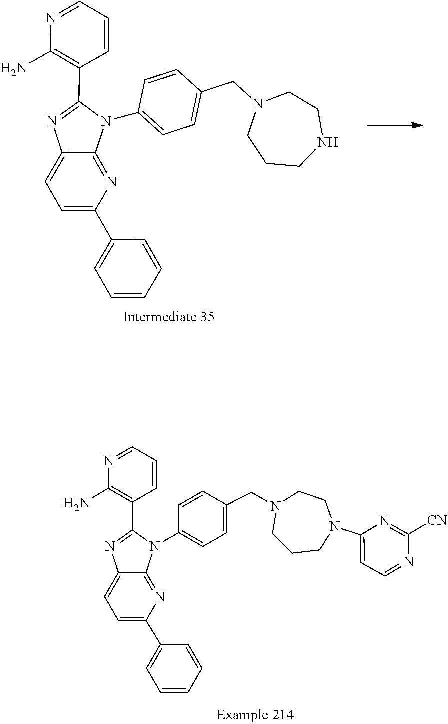 [SMILES: Nc1ncccc1-c1nc2ccc(-c3ccccc3)nc2n1-c1ccc(CN2CCCNCC2)cc1 ; N#Cc1nccc(N2CCCN(Cc3ccc(-n4c(-c5cccnc5N)nc5ccc(-c6ccccc6)nc54)cc3)CC2)n1]

Intermediate 35 (70 mg, 147 μmol), 4-chloropyrimidine-2-carbonitrile (20.5 mg, 147 μmol) and DIEA (95.1 mg, 736 μmol) were taken up into a microwave tube in NMP (3 mL). The sealed tube was heated at 160° C. for 1 hr under microwave. The reaction mixture was quenched with H₂O (5 mL) at 0° C., and then diluted with CH₂Cl₂ (10 mL) and extracted with CH₂Cl₂ (15 mL×2). The combined organic layers were washed with brine (5 mL), dried over Na₂SO₄, filtered, and concentrated under reduced pressure to give a residue. The residue was purified by prep-TLC (CH₂Cl₂:MeOH=10:1), 4-(4-(4-(2-(2-Aminopyridin-3-yl)-5-phenyl-3H-imidazo[4,5-b]pyridin-3-yl)benzyl)-1,4-diazepan-1-yl)pyrimidine-2-carbonitrile (Example 214, 8.1 mg, yield: 9.5%) was obtained as a light-yellow solid. MS: m/z=579.2 [M+H]⁺. ¹H NMR (400 MHz, Methanol-d₄) 8.24-8.10 (m, 2H), 8.07-8.01 (m, 2H), 7.99-7.99 (m, 1H), 7.95-7.91 (m, 1H), 7.52 (d, J=8.0 Hz, 2H), 7.46-7.40 (m, 4H), 7.40-7.29 (m, 2H), 6.89-6.80 (m, 1H), 6.52-6.41 (m, 1H), 4.02-3.91 (m, 2H), 3.77 (s, 2H), 3.68-3.59 (m, 2H), 2.88-2.82 (m, 2H), 2.75-2.68 (m, 2H), 2.04-1.95 (m, 2H).

### Example 215: 6-(4-(4-(2-(2-aminopyridin-3-yl)-5-phenyl-3H-imidazo[4,5-b]pyridin-3-yl)benzyl)-1,4-diazepan-1-yl)pyrimidine-4-carbonitrile

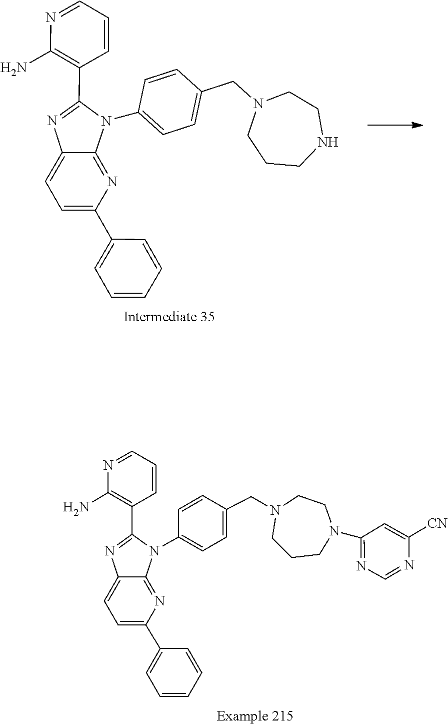 [SMILES: Nc1ncccc1-c1nc2ccc(-c3ccccc3)nc2n1-c1ccc(CN2CCCNCC2)cc1 ; N#Cc1cc(N2CCCN(Cc3ccc(-n4c(-c5cccnc5N)nc5ccc(-c6ccccc6)nc54)cc3)CC2)ncn1]

Intermediate 35 (80 mg, 168 μmol), 6-chloropyrimidine-4-carbonitrile (23.5 mg, 168 μmol) and DIEA (109 mg, 841 μmol) were taken up into a microwave tube in NMP (3 mL). The sealed tube was heated at 130° C. for 1 hr under microwave. The reaction mixture was quenched with H₂O (5 mL) at 0° C., and then diluted with CH₂Cl₂ (10 mL) and extracted with CH₂Cl₂ (15 mL×2). The combined organic layers were washed with brine (15 mL), dried over Na₂SO₄, filtered, and concentrated under reduced pressure to give a residue. The residue was purified by prep-TLC (CH₂Cl₂:MeOH=10:1), 6-(4-(4-(2-(2-aminopyridin-3-yl)-5-phenyl-3H-imidazo[4,5-b]pyridin-3-yl)benzyl)-1,4-diazepan-1-yl)pyrimidine-4-carbonitrile (Example 215, 14.0 mg, yield: 14.4%) was obtained as a yellow solid. MS: m/z=579.3 [M+H]⁺. ¹H NMR (400 MHz, Dimethylsulfoxide-d₆) 8.56 (d, J=5.6 Hz, 1H), 8.27 (d, J=8.4 Hz, 1H), 8.08-7.96 (m, 4H), 7.49-7.37 (m, 8H), 7.13 (d, J=7.6 Hz, 1H), 7.05 (br s, 2H), 6.41-6.29 (m, 1H), 3.93-3.83 (m, 2H), 3.73 (d, J=8.0 Hz, 2H), 3.68-3.59 (m, 2H), 2.77-2.72 (m, 2H), 2.63-2.58 (m, 2H), 1.93-1.82 (m, 2H).

### Example 216: 4-((1R,4R)-5-(4-(2-(2-Aminopyridin-3-yl)-5-phenyl-3H-imidazo[4,5-b]pyridin-3-yl)benzyl)-2,5-diazabicyclo[2.2.1]heptan-2-yl)pyrimidine-2-carbonitrile

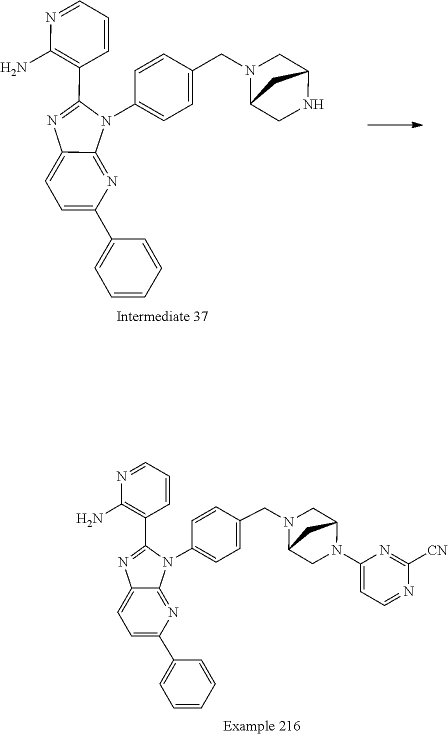 [SMILES: Nc1ncccc1-c1nc2ccc(-c3ccccc3)nc2n1-c1ccc(CN2C[C@H]3C[C@@H]2CN3)cc1 ; N#Cc1nccc(N2C[C@H]3C[C@@H]2CN3Cc2ccc(-n3c(-c4cccnc4N)nc4ccc(-c5ccccc5)nc43)cc2)n1]

To a solution of Intermediate 37 (80 mg, 169 μmol) and 4-chloropyrimidine-2-carbonitrile (23.6 mg, 169 μmol) in NMP (2 mL) was added DIEA (109 mg, 845 μmol). The mixture was stirred under microwave at 130° C. for 0.5 hr. The reaction was filtered and concentrated under reduced pressure to give a residue. The crude product was purified by prep-HPLC (column: Waters xbridge 150*25 mm 10 μm; mobile phase: [water (NH₄HCO₃)-ACN]; B %: 48%-78%, 10 min), 4-((1R,4R)-5-(4-(2-(2-aminopyridin-3-yl)-5-phenyl-3H-imidazo[4,5-b]pyridin-3-yl)benzyl)-2,5-diazabicyclo[2.2.1]heptan-2-yl)pyrimidine-2-carbonitrile (Example 216, 22 mg, yield: 23%) was obtained as a yellow solid. MS: m/z=577.3 [M+H]⁺. ¹H NMR (400 MHz, Dimethylsulfoxide-d₆) δ 8.27-8.18 (m, 2H), 8.03-7.97 (m, 4H), 7.52-7.37 (m, 8H), 7.16 (dd, J=7.6, 1.6 Hz, 1H), 6.99 (br s, 2H), 6.41-6.37 (m, 1H), 4.89-4.68 (m, 1H), 3.84 (s, 2H), 3.70-3.67 (m, 2H), 3.56-3.53 (m, 1H), 3.42-3.41 (m, 1H), 2.94 (t, J=8.0 Hz, 1H), 2.07-1.97 (m, 1H), 1.86-1.78 (m, 1H).

### Example 217: 2-((1R,4R)-5-(4-(2-(2-Aminopyridin-3-yl)-5-phenyl-3H-imidazo[4,5-b]pyridin-3-yl)benzyl)-2,5-diazabicyclo[2.2.1]heptan-2-yl)pyrimidine-4-carbonitrile

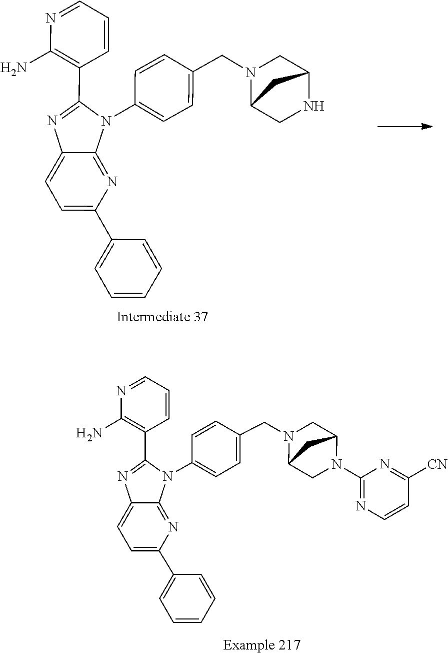 [SMILES: Nc1ncccc1-c1nc2ccc(-c3ccccc3)nc2n1-c1ccc(CN2C[C@H]3C[C@@H]2CN3)cc1 ; N#Cc1ccnc(N2C[C@H]3C[C@@H]2CN3Cc2ccc(-n3c(-c4cccnc4N)nc4ccc(-c5ccccc5)nc43)cc2)n1]

To a solution of Intermediate 37 (80 mg, 169 μmol) and 2-chloropyrimidine-4-carbonitrile (23.6 mg, 169 μmol) in NMP (2 mL) was added DIEA (109 mg, 845 μmol). The mixture was stirred under microwave at 130° C. for 0.5 hr. The reaction was filtered and concentrated under reduced pressure to give a residue. The crude product was purified by prep-HPLC (column: Waters xbridge 150*25 mm 10 μm; mobile phase: [water (NH₄HCO₃)-ACN]; B %: 48%-78%, 10 min), 2-((1R,4R)-5-(4-(2-(2-aminopyridin-3-yl)-5-phenyl-3H-imidazo[4,5-b]pyridin-3-yl)benzyl)-2,5-diazabicyclo[2.2.1]heptan-2-yl)pyrimidine-4-carbonitrile (Example 217, 27.2 mg, yield: 28%) was obtained as a yellow solid. MS: m/z=577.4 [M+H]⁺. ¹H NMR (400 MHz, Dimethylsulfoxide-d₆) δ 8.63-8.59 (m, 1H), 8.26 (d, J=8.4 Hz, 1H), 8.05-7.95 (m, 4H), 7.51-7.37 (m, 7H), 7.16 (dd, J=8.0, 1.6 Hz, 1H), 7.12 (d, J=4.4 Hz, 1H), 6.99 (br s, 2H), 6.39 (dd, J=7.6, 5.2 Hz, 1H), 4.81-4.73 (m, 1H), 3.87 (s, 2H), 3.69-3.62 (m, 2H), 3.45-3.40 (m, 2H), 2.96-2.94 (m, 1H), 1.99-1.97 (m, 1H), 1.84-1.82 (m, 1H).

### Example 218: 6-((1R,4R)-5-(4-(2-(2-Aminopyridin-3-yl)-5-phenyl-3H-imidazo[4,5-b]pyridin-3-yl)benzyl)-2,5-diazabicyclo[2.2.]heptan-2-yl)pyrimidine-4-carbonitrile

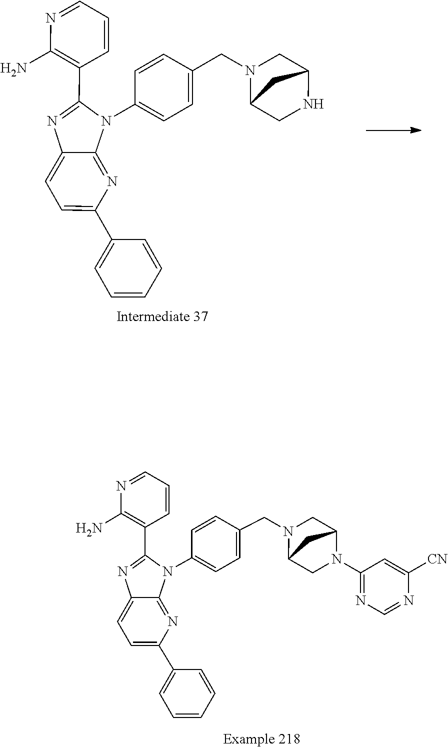 [SMILES: Nc1ncccc1-c1nc2ccc(-c3ccccc3)nc2n1-c1ccc(CN2C[C@H]3C[C@@H]2CN3)cc1 ; N#Cc1cc(N2C[C@H]3C[C@@H]2CN3Cc2ccc(-n3c(-c4cccnc4N)nc4ccc(-c5ccccc5)nc43)cc2)ncn1]

To a solution of Intermediate 37 (80 mg, 169 μmol) and 6-chloropyrimidine-4-carbonitrile (23.6 mg, 169 μmol) in NMP (2 mL) was added DIEA (109 mg, 845 μmol). The mixture was stirred under microwave at 130° C. for 0.5 hr. The reaction was filtered and concentrated under reduced pressure to give a residue. The crude product was purified by prep-HPLC (column: Waters xbridge 150*25 mm 10 μm; mobile phase: [water (NH₄HCO₃)-ACN]; B %: 50%-80%, 10 min), 6-((1R,4R)-5-(4-(2-(2-aminopyridin-3-yl)-5-phenyl-3H-imidazo[4,5-b]pyridin-3-yl)benzyl)-2,5-diazabicyclo[2.2.1]heptan-2-yl)pyrimidine-4-carbonitrile (Example 218, 11.4 mg, yield: 11.7%) was obtained as a yellow solid. MS: m/z=577.4 [M+H]r. ¹H NMR (400 MHz, Dimethylsulfoxide-d₆) δ 8.52 (d, J=12.4 Hz, 1H), 8.26 (d, J=8.4 Hz, 1H), 8.04-8.00 (m, 3H), 7.99-7.97 (m, 1H), 7.51-7.41 (m, 7H), 7.17-7.15 (m, 2H), 6.99 (br s, 2H), 6.39 (dd, J=7.6, 4.8 Hz, 1H), 4.94-4.71 (m, 1H), 3.84-3.82 (m, 2H), 3.72-3.67 (m, 2H), 3.60-3.58 (m, 1H), 3.43-3.42 (m, 1H), 2.95-2.90 (m, 1H), 2.04-1.97 (m, 1H), 1.8-1.78 (m, 1H).

### Example 219: 4-(5-(4-(2-(2-Aminopyridin-3-yl)-5-phenyl-3H-imidazo[4,5-b]pyridin-3-yl)benzyl)-2,5-diazabicyclo[2.2.2]octan-2-yl)pyrimidine-2-carbonitrile

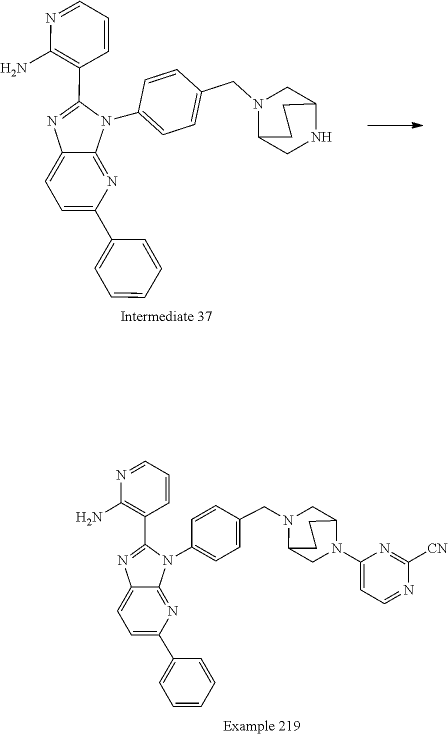 [SMILES: Nc1ncccc1-c1nc2ccc(-c3ccccc3)nc2n1-c1ccc(CN2CC3CCC2CN3)cc1 ; N#Cc1nccc(N2CC3CCC2CN3Cc2ccc(-n3c(-c4cccnc4N)nc4ccc(-c5ccccc5)nc43)cc2)n1]

Intermediate 38 (100 mg, 205 μmol), 4-chloropyrimidine-2-carbonitrile (28.6 mg, 206 μmol) and DIEA (132 mg, 1.0 mmol) were taken up into a microwave tube in NMP (2 mL). The sealed tube was heated at 130° C. for 1 hr under microwave. The reaction mixture was quenched with H₂O (5 mL) at 0° C., and then diluted with 10 mL CH₂Cl₂ (10 mL) and extracted with CH₂Cl₂ (15 mL×2). The combined organic layers were washed with 15 mL aqueous NaCl, dried over Na₂SO₄, filtered, and concentrated under reduced pressure to give a residue. After purified by prep-HPLC (column: Waters xbridge 150×25 mm 10 μm; mobile phase: [water (NH₄HCO₃)-ACN]; B %: 52%-82%, 8 min), 4-(5-(4-(2-(2-aminopyridin-3-yl)-5-phenyl-3H-imidazo[4,5-b]pyridin-3-yl)benzyl)-2,5-diazabicyclo[2.2.2]octan-2-yl)pyrimidine-2-carbonitrile (Example 219, 24.4 mg, yield: 20%) was obtained as a yellow lyophilized powder. MS: m/z=591.3 [M+H]⁺. ¹H NMR (400 MHz, Dimethylsulfoxide-d₆) 8.28-8.18 (m, 2H), 8.04-7.97 (m, 4H), 7.53 (d, J=8.4 Hz, 2H), 7.49-7.43 (m, 4H), 7.42-7.37 (m, 1H), 7.16 (dd, J=7.6, 1.6 Hz, 1H), 7.01 (s, 2H), 6.76 (d, J=6.4 Hz, 1H), 6.42-6.35 (m, 1H), 4.76-4.73 (m, 0.6H), 4.24-4.21 (m, 0.4H), 3.89-3.80 (m, 3H), 3.49-3.52 (m, 1H), 3.02-3.07 (m, 1H), 2.94-2.88 (m, 2H), 2.20-2.03 (m, 1H), 1.89-1.80 (m, 2H), 1.71-1.63 (m, 1H).

### Example 220: 2-(5-(4-(2-(2-Aminopyridin-3-yl)-5-phenyl-3H-imidazo[4,5-b]pyridin-3-yl)benzyl)-2,5-diazabicyclo[2.2.2]octan-2-yl)pyrimidine-4-carbonitrile

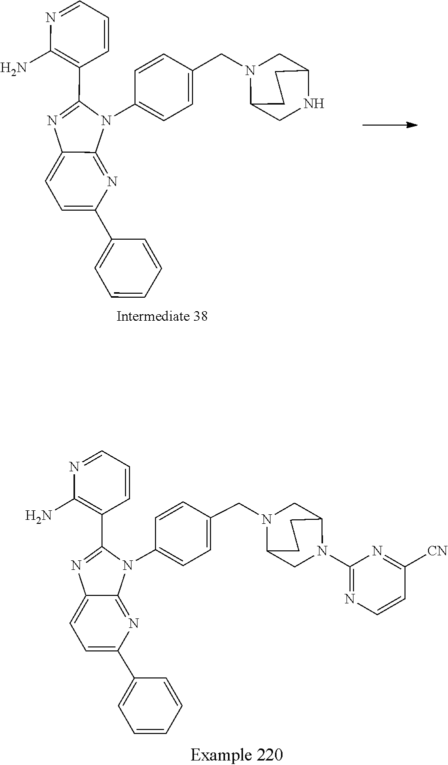 [SMILES: Nc1ncccc1-c1nc2ccc(-c3ccccc3)nc2n1-c1ccc(CN2CC3CCC2CN3)cc1 ; N#Cc1ccnc(N2CC3CCC2CN3Cc2ccc(-n3c(-c4cccnc4N)nc4ccc(-c5ccccc5)nc43)cc2)n1]

Intermediate 38 (100 mg, 205 μmol), 4-chloropyrimidine-2-carbonitrile (28.6 mg, 206 μmol) and DIEA (132 mg, 1.0 mmol) were taken up into a microwave tube in NMP (2 mL). The sealed tube was heated at 130° C. for 1 hr under microwave. The reaction mixture was quenched with H₂O (5 mL) at 0° C., and then diluted with CH₂Cl₂ (10 mL) and extracted with CH₂Cl₂ (15 mL×2). The combined organic layers were washed with brine (15 mL), dried over Na₂SO₄, filtered, and concentrated under reduced pressure to give a residue. After purified by prep-HPLC (column: Waters xbridge 150×25 mm 10 μm; mobile phase: [water (NH₄HCO₃)-ACN]; B %: 55%-85%, 10 min), 2-(5-(4-(2-(2-Aminopyridin-3-yl)-5-phenyl-3H-imidazo[4,5-b]pyridin-3-yl)benzyl)-2,5-diazabicyclo[2.2.2]octan-2-yl)pyrimidine-4-carbonitrile (Example 220, 18.9 mg, yield: 15%) was obtained as a yellow lyophilized powder. MS: m/z=591.4 [M+H]⁺. ¹H NMR (400 MHz, Dimethylsulfoxide-d₆) 8.68-8.54 (m, 1H), 8.27 (d, J=8.0 Hz, 1H), 8.06-7.95 (m, 4H), 7.53 (d, J=8.4 Hz, 2H), 7.49-7.43 (m, 4H), 7.42-7.37 (m, 1H), 7.16 (dd, J=7.6, 2.0 Hz, 1H), 7.11 (d, J=4.8 Hz, 1H), 7.02 (s, 2H), 6.39 (dd, J=7.6, 4.8 Hz, 1H), 4.73-4.55 (m, 1H), 3.92-3.80 (m, 3H), 3.49-3.44 (m, 1H), 3.03-3.05 (m, 1H), 2.96-2.85 (m, 2H), 2.16-2.06 (m, 1H), 1.89-1.79 (m, 2H), 1.71-1.61 (m, 1H).

### Example 221: 6-(5-(4-(2-(2-Aminopyridin-3-yl)-5-phenyl-3H-imidazo[4,5-b]pyridin-3-yl)benzyl)-2,5-diazabicyclo[2.2.2]octan-2-yl)pyrimidine-4-carbonitrile

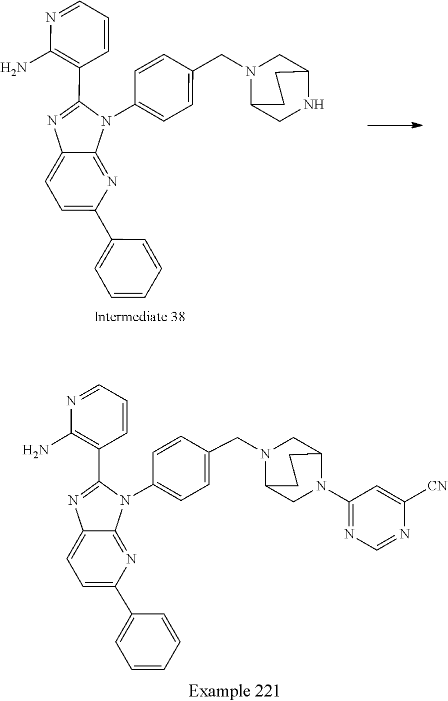 [SMILES: Nc1ncccc1-c1nc2ccc(-c3ccccc3)nc2n1-c1ccc(CN2CC3CCC2CN3)cc1 ; N#Cc1cc(N2CC3CCC2CN3Cc2ccc(-n3c(-c4cccnc4N)nc4ccc(-c5ccccc5)nc43)cc2)ncn1]

Intermediate 38 (100 mg, 205 μmol), 4-chloropyrimidine-2-carbonitrile (28.6 mg, 206 μmol) and DIEA (132 mg, 1.0 mmol) were taken up into a microwave tube in NMP (2 mL). The sealed tube was heated at 130° C. for 1 hr under microwave. The reaction mixture was quenched with H₂O (5 mL) at 0° C., and then diluted with CH₂Cl₂ (10 mL) and extracted with CH₂Cl₂ (15 mL×2). The combined organic layers were washed with brine (15 mL), dried over Na₂SO₄, filtered and concentrated under reduced pressure to give a residue. After purified by prep-HPLC (column: Waters xbridge 150×25 mm 10 μm; mobile phase: [water (NH₄HCO₃)-ACN]; B %: 48-78%, 10 min), 6-(5-(4-(2-(2-aminopyridin-3-yl)-5-phenyl-3H-imidazo[4,5-b]pyridin-3-yl)benzyl)-2,5-diazabicyclo[2.2.2]octan-2-yl)pyrimidine-4-carbonitrile (Example 221, 17.8 mg, yield: 14%) was obtained as a yellow lyophilized powder. MS: m/z=591.3 [M+H]⁺. ¹H NMR (400 MHz, Dimethylsulfoxide-d₆) 8.60-8.48 (m, 1H), 8.27 (d, J=8.4 Hz, 1H), 8.05-7.97 (m, 4H), 7.52 (d, J=8.4 Hz, 2H), 7.49-7.43 (m, 4H), 7.42-7.37 (m, 1H), 7.24-7.10 (m, 2H), 7.01 (s, 2H), 6.40-6.36 (m, 1H), 4.25-4.85 (m, 1H), 3.90-3.78 (m, 3H), 3.56-3.49 (m, 1H), 3.05-3.07 (m, 1H), 2.94-2.86 (m, 2H), 2.17-2.08 (m, 1H), 1.90-1.79 (m, 2H), 1.71-1.63 (m, 1H).

### Example 222: 4-((1-((4-(2-(2-Aminopyridin-3-yl)-5-phenyl-3H-imidazo[4,5-b]pyridin-3-yl)phenyl)methyl-d2)piperidin-4-yl)amino)pyrimidine-2-carbonitrile

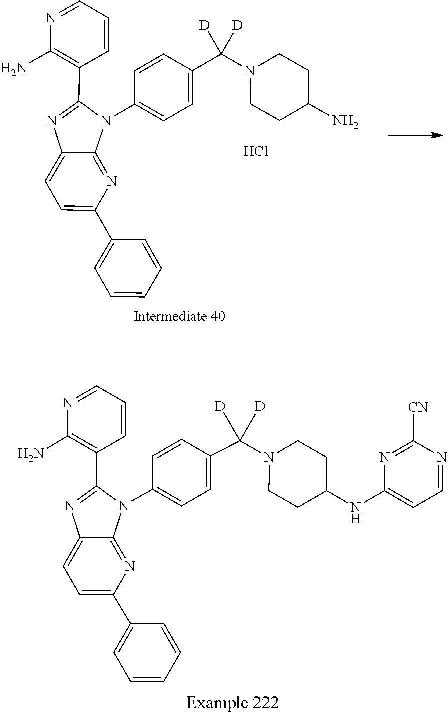 [SMILES: [2H]C([2H])(c1ccc(-n2c(-c3cccnc3N)nc3ccc(-c4ccccc4)nc32)cc1)N1CCC(N)CC1 ; Cl ; [2H]C([2H])(c1ccc(-n2c(-c3cccnc3N)nc3ccc(-c4ccccc4)nc32)cc1)N1CCC(Nc2ccnc(C#N)n2)CC1]

To a solution of Intermediate 40 (50.0 mg, 105 μmol) in NMP (1 mL) were added 4-chloropyrimidine-2-carbonitrile (16.1 mg, 115 μmol) and DIEA (67.6 mg, 523 μmol, 91.2 μL) under N₂ atmosphere. The mixture was stirred at 130° C. for 30 min under microwave. Water (5 mL) was added to the mixture and the mixture was extracted with EtOAc (3×5 mL). The combined organic layers were washed with brine (10 mL), dried over Na₂SO₄, filtered, and concentrated. The residue was purified by prep-HPLC (column: Phenomenex C18 80*40 mm*3 μm; mobile phase: [water (NH₄OH+NH₄HCO₃)-ACN]; B %: 51%-81%, 8 min) to give 4-((1-((4-(2-(2-Aminopyridin-3-yl)-5-phenyl-3H-imidazo[4,5-b]pyridin-3-yl)phenyl)methyl-d2)piperidin-4-yl)amino)pyrimidine-2-carbonitrile (Example 222, 20.3 mg, yield: 33%) was obtained as an off-white solid. MS: m/z=581.3 [M+H]⁺. ¹H NMR (400 MHz, Methanol-d₄) δ 8.24 (d, J=8.4 Hz, 1H), 8.10 (d, J=6.0 Hz, 1H), 8.04-7.95 (m, 4H), 7.77 (d, J=8.4 Hz, 2H), 7.67 (d, J=8.0 Hz, 2H), 7.50-7.38 (m, 4H), 6.67 (s, 1H), 6.59 (dd, J=7.6, 5.6 Hz, 1H), 4.25-4.19 (m, 1H), 3.62-3.58 (m, 2H), 3.38-3.33 (m, 1H), 3.29-3.19 (m, 1H), 2.29-2.26 (m, 2H), 1.86-1.80 (m, 2H).

### Example 223: 4-((1-(4-(2-(2-aminopyridin-3-yl)-5-phenyl-3H-imidazo[4,5-b]pyridin-3-yl)benzyl)piperidin-4-yl)(methyl)amino)-1,3,5-triazine-2-carbonitrile

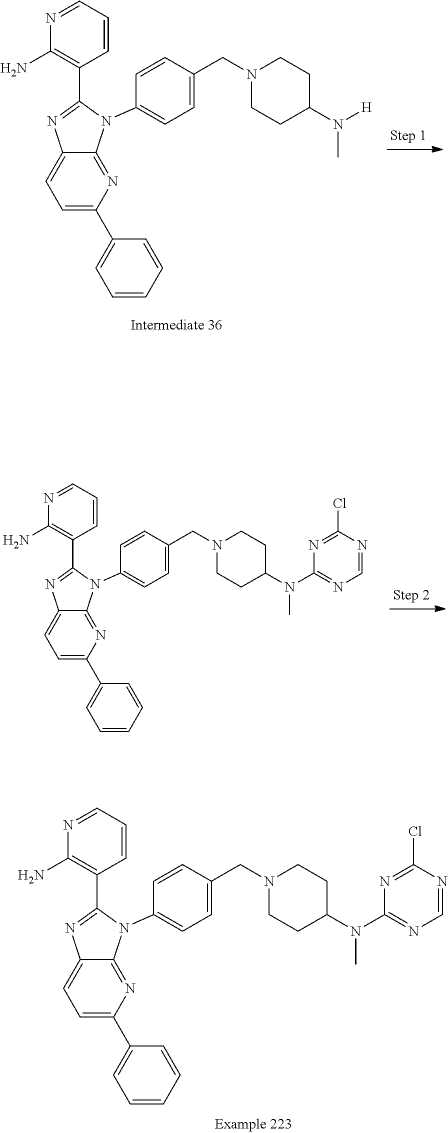 [SMILES: CNC1CCN(Cc2ccc(-n3c(-c4cccnc4N)nc4ccc(-c5ccccc5)nc43)cc2)CC1 ; CN(c1ncnc(Cl)n1)C1CCN(Cc2ccc(-n3c(-c4cccnc4N)nc4ccc(-c5ccccc5)nc43)cc2)CC1 ; CN(c1ncnc(Cl)n1)C1CCN(Cc2ccc(-n3c(-c4cccnc4N)nc4ccc(-c5ccccc5)nc43)cc2)CC1]

### Step 1: N-(1-(4-(2-(2-Aminopyridin-3-yl)-5-phenyl-3H-imidazo[4,5-b]pyridin-3-yl)benzyl)piperidin-4-yl)-4-chloro-N-methyl-1,3,5-triazin-2-amine

To a solution of Intermediate 36 (110 mg, 225 μmol) in THF (15 mL) was added DIEA (87.1 mg, 674 μmol). The mixture was stirred at 0° C. for 10 min. Then 2,4-dichloro-1,3,5-triazine (37.1 mg, 247 μmol) in THF (2 mL) was added to the mixture. The mixture was stirred at 0° C. for 1 hr. The reaction mixture was concentrated, and purified by silica gel flash chromatography (Eluent of 0˜100% EtOAc in petroleum ether) to give N-(1-(4-(2-(2-aminopyridin-3-yl)-5-phenyl-3H-imidazo[4,5-b]pyridin-3-yl)benzyl)piperidin-4-yl)-4-chloro-N-methyl-1,3,5-triazin-2-amine. (40 mg, yield: 30%) as a light-yellow solid. MS: m/z=603.2 [M+H]⁺.

### Step 2: 4-((1-(4-(2-(2-Aminopyridin-3-yl)-5-phenyl-3H-imidazo[4,5-b]pyridin-3-yl)benzyl)piperidin-4-yl)(methyl)amino)-1,3,5-triazine-2-carbonitrile

To a solution of N-(1-(4-(2-(2-aminopyridin-3-yl)-5-phenyl-3H-imidazo[4,5-b]pyridin-3-yl)benzyl)piperidin-4-yl)-4-chloro-N-methyl-1,3,5-triazin-2-amine (40 mg, 66.3 μmol) in DMSO (1 mL) were added KCN (13.0 mg, 199 μmol) and DABCO (1.5 mg, 13.3 μmol). The mixture was stirred at 25° C. for 1 hr. The reaction mixture was poured into water (5 mL). The resulting mixture was extracted with CH₂Cl₂ (5 mL×2). The combined organic layers were washed with brine (5 ml) and dried over anhydrous Na₂SO₄, filtered and concentrated under reduced pressure. The residue was purified by prep-HPLC (column: Waters xbridge 150*25 mm 10 um; mobile phase: [water (NH₄HCO₃)-ACN]; gradient: 53%-83% B over 14 min) to give 4-((1-(4-(2-(2-aminopyridin-3-yl)-5-phenyl-3H-imidazo[4,5-b]pyridin-3-yl)benzyl)piperidin-4-yl)(methyl)amino)-1,3,5-triazine-2-carbonitrile (Example 223, 14.0 mg, yield: 36%) as a yellow solid. MS: m/z=594.2 [M+H]⁺. ¹H NMR (400 MHz, Acetonitrile-d₃) δ 8.58 (d, J=6.8 Hz, 1H), 8.20 (d, J=8.4 Hz, 1H), 8.11-7.99 (m, 3H), 7.93 (d, J=8.4 Hz, 1H), 7.57-7.52 (m, 2H), 7.51-7.40 (m, 5H), 7.18 (dd, J=7.6, 1.2 Hz, 1H), 6.60 (br s, 2H), 6.41 (dd, J=7.6, 4.8 Hz, 1H), 4.63-4.53 (m, 1H), 3.66 (d, J=4.4 Hz, 2H), 3.11-3.07 (m, 3H), 3.07-3.01 (m, 2H), 2.23-2.19 (m, 2H), 1.95-1.88 (m, 2H), 1.72-1.69 (m, 2H).

### Example 224: (S)-4-((1-(4-(2-(2-Aminopyridin-3-yl)-5-phenyl-3H-imidazo[4,5-b]pyridin-3-yl)benzyl)piperidin-3-yl)amino)-1,3,5-triazine-2-carbonitrile

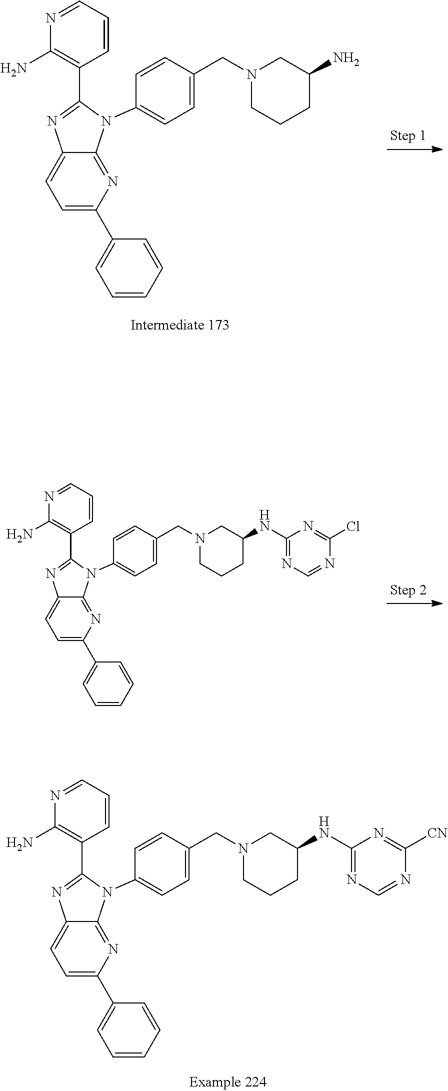 [SMILES: Nc1ncccc1-c1nc2ccc(-c3ccccc3)nc2n1-c1ccc(CN2CCC[C@H](N)C2)cc1 ; Nc1ncccc1-c1nc2ccc(-c3ccccc3)nc2n1-c1ccc(CN2CCC[C@H](Nc3ncnc(Cl)n3)C2)cc1 ; N#Cc1ncnc(N[C@H]2CCCN(Cc3ccc(-n4c(-c5cccnc5N)nc5ccc(-c6ccccc6)nc54)cc3)C2)n1]

### Step 1 (S)—N-(1-(4-(2-(2-Aminopyridin-3-yl)-5-phenyl-3H-imidazo[4,5-b]pyridin-3-yl)benzyl)piperidin-3-yl)-4-chloro-1,3,5-triazin-2-amine

To a solution of Intermediate 173 (50 mg, 105 μmol) in THF (8 mL) was added DIEA (40.7 mg, 315 μmol). The 2,4-dichloro-1,3,5-triazine (17.3 mg, 116 μmol) in THF (2 mL) was added into the mixture at 0° C. The mixture was stirred at 0° C. for 0.5 hr. The mixture was concentrated under reduced pressure. After purified by prep-TLC (EtOAc), (S)—N-(1-(4-(2-(2-aminopyridin-3-yl)-5-phenyl-3H-imidazo[4,5-b]pyridin-3-yl)benzyl)piperidin-3-yl)-4-chloro-1,3,5-triazin-2-amine (14.9 mg, yield: 23%) was obtained as a light-yellow solid. MS: m/z=589.3 [M+H]⁺. ¹H NMR (400 MHz, Methanol-d₄) δ 8.32-8.26 (m, 1H), 8.18 (d, J=8.4 Hz, 1H), 8.05-7.99 (m, 2H), 7.98-7.91 (m, 2H), 7.59-7.50 (m, 2H), 7.46-7.33 (m, 5H), 7.31-7.26 (m, 1H), 6.42-6.37 (m, 1H), 4.13-4.09 (m, 1H), 3.73-3.62 (m, 2H), 2.96-2.91 (m, 1H), 2.81-2.72 (m, 1H), 2.33-2.09 (m, 2H), 1.93-1.90 (m, 1H), 1.84-1.77 (m, 1H), 1.73-1.63 (m, 1H), 1.52-1.38 (m, 1H).

### Step 2: (S)-4-((1-(4-(2-(2-Aminopyridin-3-yl)-5-phenyl-3H-imidazo[4,5-b]pyridin-3-yl)benzyl)piperidin-3-yl)amino)-1,3,5-triazine-2-carbonitrile

To a solution of (S)—N-(1-(4-(2-(2-aminopyridin-3-yl)-5-phenyl-3H-imidazo[4,5-b]pyridin-3-yl)benzyl)piperidin-3-yl)-4-chloro-1,3,5-triazin-2-amine (50 mg, 84.9 μmol) in DMSO (1 mL) were added KCN (60 mg, 921 μmol) and DABCO (1.9 mg, 17 μmol). The mixture was stirred at 25° C. for 2 hr. The reaction mixture was poured into H₂O (10 mL). The resulting mixture was extracted with EtOAc (10 mL×2). The combined organic layers were washed with brine (10 mL) and dried over anhydrous Na₂SO₄, filtered and concentrated. After purified by silica gel flash chromatography (Eluent of 0˜8% MeOH in CH₂Cl₂), (S)-4-((1-(4-(2-(2-Aminopyridin-3-yl)-5-phenyl-3H-imidazo[4,5-b]pyridin-3-yl)benzyl)piperidin-3-yl)amino)-1,3,5-triazine-2-carbonitrile (Example 224, 10 mg, yield: 18%) as a light-yellow solid. MS: m/z=580.3 [M+H]⁺. ¹H NMR (400 MHz, Dimethylsulfoxide-d₆) δ 8.86 (dd, J=8.4, 2.8 Hz, 1H), 8.64 (d, J=16.0 Hz, 1H), 8.27 (d, J=8.0 Hz, 1H), 8.04-7.99 (m, 3H), 7.97-7.96 (m, 1H), 7.52-7.48 (m, 2H), 7.46-7.43 (m, 4H), 7.40-7.36 (m, 1H), 7.15-7.12 (m, 1H), 7.03 (d, J=8.4 Hz, 2H), 6.33-6.27 (m, 1H), 4.02-3.93 (m, 1H), 3.62 (d, J=8.0 Hz, 2H), 2.91-2.84 (m, 1H), 2.74-2.69 (m, 1H), 2.03-1.98 (m, 2H), 1.87-1.82 (m, 1H), 1.76-1.71 (m, 1H), 1.57-1.50 (m, 1H), 1.36-1.32 (m, 1H).

### Example 225: (R)-4-(4-(4-(2-(2-Aminopyridin-3-yl)-5-phenyl-3H-imidazo[4,5-b]pyridin-3-yl)benzyl)-2-methylpiperazin-1-yl)pyrimidine-2-carbonitrile

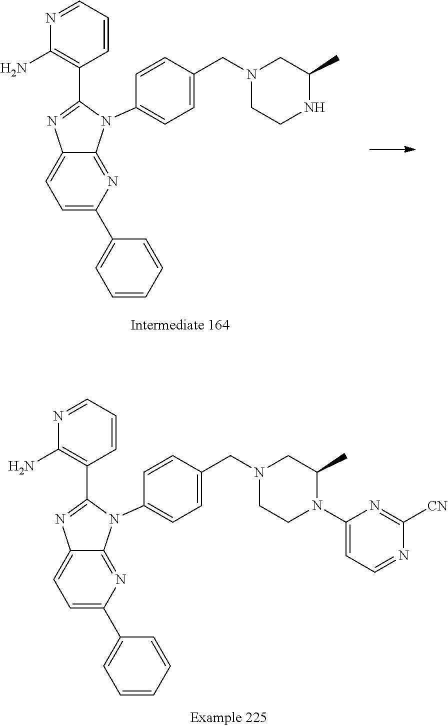 [SMILES: C[C@@H]1CN(Cc2ccc(-n3c(-c4cccnc4N)nc4ccc(-c5ccccc5)nc43)cc2)CCN1 ; C[C@@H]1CN(Cc2ccc(-n3c(-c4cccnc4N)nc4ccc(-c5ccccc5)nc43)cc2)CCN1c1ccnc(C#N)n1]

A mixture of Intermediate 164 (100 mg, 210 μmol), 4-chloropyrimidine-2-carbonitrile (29.3 mg, 210 μmol) and DIEA (136 mg, 1.05 mmol) in NMP (2 mL) was taken up into a microwave tube. The sealed tube was heated at 130° C. for 1 hr under microwave. The reaction mixture was quenched with H₂O (5 mL) at 0° C., and then diluted with CH₂Cl₂ (10 mL) and extracted with CH₂Cl₂ (15 mL×2). The combined organic layers were washed with brine (15 mL), dried over Na₂SO₄, filtered and concentrated under reduced pressure. After purified by prep-HPLC (column: Welch Xtimate C18 150×25 mm×5 μm; mobile phase: [water (HCl)-ACN]; B %: 13%-43%, 10 min), (R)-4-(4-(4-(2-(2-aminopyridin-3-yl)-5-phenyl-3H-imidazo[4,5-b]pyridin-3-yl)benzyl)-2-methylpiperazin-1-yl)pyrimidine-2-carbonitrile (Example 225, 14.5 mg HCl salt, yield: 12%) was obtained as a yellow solid. MS: m/z=579.3 [M+H]⁺. ¹H NMR (400 MHz, Methanol-d₄) δ 8.36-8.31 (m, 2H), 8.05-8.01 (m, 4H), 7.92 (d, J=8.4 Hz, 2H), 7.87 (dd, J=7.6, 1.2 Hz, 1H), 7.76 (d, J=8.4 Hz, 2H), 7.47-7.41 (m, 3H), 7.09 (d, J=6.4 Hz, 1H), 6.87 (dd, J=7.6, 6.4 Hz, 1H), 3.72 (d, J=11.6 Hz, 1H), 3.63-3.55 (m, 2H), 3.49-3.36 (m, 2H), 3.35-3.31 (m, 2H), 2.86-2.66 (m, 2H), 1.46 (d, J=6.8 Hz, 3H).

### Example 226: (R)-2-(4-(4-(2-(2-Aminopyridin-3-yl)-5-phenyl-3H-imidazo[4,5-b]pyridin-3-yl)benzyl)-2-methylpiperazin-1-yl)pyrimidine-4-carbonitrile

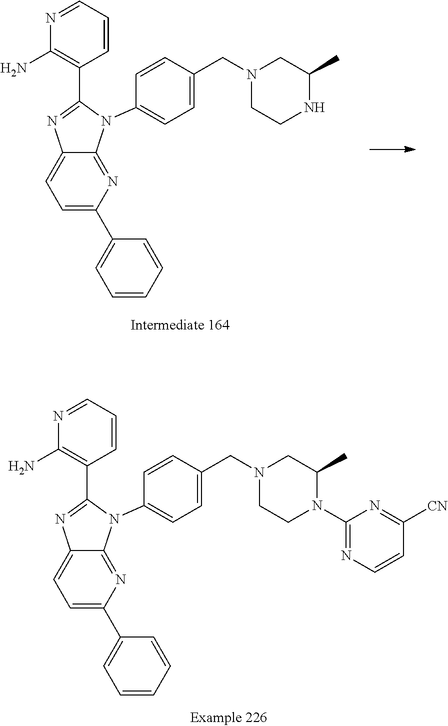 [SMILES: C[C@@H]1CN(Cc2ccc(-n3c(-c4cccnc4N)nc4ccc(-c5ccccc5)nc43)cc2)CCN1 ; C[C@@H]1CN(Cc2ccc(-n3c(-c4cccnc4N)nc4ccc(-c5ccccc5)nc43)cc2)CCN1c1nccc(C#N)n1]

A mixture of Intermediate 164 (100 mg, 210 μmol), 2-chloropyrimidine-4-carbonitrile (29.3 mg, 210 μmol) and DIEA (136 mg, 1.05 mmol) in NMP (3 mL) was taken up into a microwave tube. The sealed tube was heated at 130° C. for 1 hr under microwave. The reaction mixture was quenched with H₂O (5 mL) at 0° C., and then diluted with CH₂Cl₂ (10 mL) and extracted with CH₂Cl₂ (15 mL×2). The combined organic layers were washed with brine (15 mL), dried over Na₂SO₄, filtered, and concentrated under reduced pressure. After purified by prep-HPLC (column. Welch Xtimate C18 150×25 mm×5 μm; mobile phase: [water (HCl)-ACN]; B %: 15%-45%, 10 min), (R)-2-(4-(4-(2-(2-aminopyridin-3-yl)-5-phenyl-3H-imidazo[4,5-b]pyridin-3-yl)benzyl)-2-methylpiperazin-1-yl)pyrimidine-4-carbonitrile (Example 226, 14.1 mg, yield: 11%) was obtained as a yellow solid. MS: m/z=579.3 [M+H]⁺. ¹H NMR (400 MHz, Methanol-d₄) δ 8.65 (d, J=4.8 Hz, 1H), 8.33 (d, J=8.4 Hz, 1H), 8.06-8.01 (m, 4H), 7.92 (d, J=8.0 Hz, 2H), 7.87 (d, J=7.2 Hz, 1H), 7.76 (d, J=8.0 Hz, 2H), 7.47-7.40 (m, 3H), 7.10 (d, J=4.8 Hz, 1H), 6.86 (t, J=0.2 Hz, 1H), 3.71 (d, J=10.4 Hz, 1H), 3.55 (t, J=12.4 Hz, 2H), 3.49-3.32 (m, 4H), 3.30-3.19 (m, 2H), 1.43 (d, J=7.2 Hz, 3H).

### Example 227: (R)-6-(4-(4-(2-(2-Aminopyridin-3-yl)-5-phenyl-3H-imidazo[4,5-b]pyridin-3-yl)benzyl)-2-methylpiperazin-1-yl)pyrimidine-4-carbonitrile

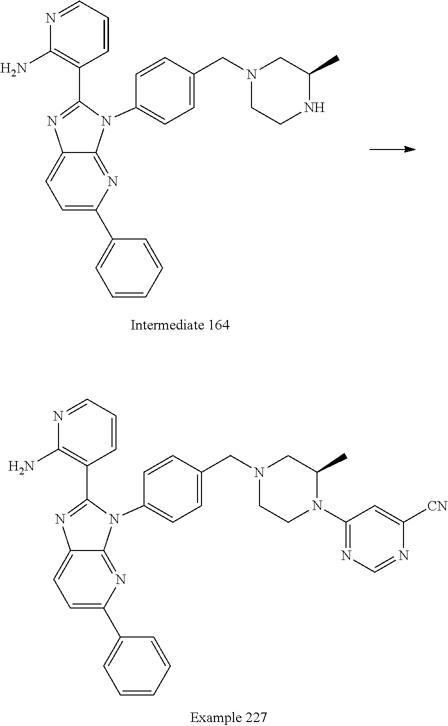 [SMILES: C[C@@H]1CN(Cc2ccc(-n3c(-c4cccnc4N)nc4ccc(-c5ccccc5)nc43)cc2)CCN1 ; C[C@@H]1CN(Cc2ccc(-n3c(-c4cccnc4N)nc4ccc(-c5ccccc5)nc43)cc2)CCN1c1cc(C#N)ncn1]

A mixture of Intermediate 164 (94.5 mg, 206 μmol), 6-chloropyrimidine-4-carbonitrile (28.6 mg, 206 μmol) and DIEA (132 mg, 1.0 mmol) in NMP (3 mL) was taken up into a microwave tube. The sealed tube was heated at 130° C. for 1 hr under microwave. The reaction mixture was quenched with H₂O (5 mL) at 0° C., and then diluted with CH₂Cl₂ (10 mL) and extracted with CH₂Cl₂ (15 mL×2). The combined organic layers were washed with brine (15 mL), dried over Na₂SO₄, filtered and concentrated under reduced pressure. After purified by prep-HPLC (column: Welch Xtimate C18 150×25 mm×5 μm; mobile phase: [water (HCl)-ACN]; B %: 12%-42%, 10 min), (R)-6-(4-(4-(2-(2-aminopyridin-3-yl)-5-phenyl-3H-imidazo[4,5-b]pyridin-3-yl)benzyl)-2-methylpiperazin-1-yl)pyrimidine-4-carbonitrile (Example 227, 19.8 mg, yield: 16%) was obtained as a yellow solid. MS: m/z=579.3 [M+H]⁺. ¹H NMR (400 MHz, Methanol-d₄) δ 8.65 (s, 1H), 8.33 (d, J=8.4 Hz, 1H), 8.05-8.02 (m, 4H), 7.93 (d, J=8.4 Hz, 2H), 7.88 (dd, J=7.6, 1.2 Hz, 1H), 7.76 (d, J=8.4 Hz, 2H), 7.47-7.42 (m, 4H), 6.87 (dd, J=7.6, 6.4 Hz, 1H), 3.76-3.69 (m, 1H), 3.63-3.55 (m, 2H), 3.51-3.32 (m, 4H), 3.30-3.22 (m, 2H), 1.46 (d, J=7.2 Hz, 3H).

### Example 228: 2-(4-(4-(2-(2-Aminopyridin-3-yl)-5-phenyl-3H-imidazo[4,5-b]pyridin-3-yl)benzyl)-1,4-diazepan-1-yl)pyrimidine-4-carbonitrile

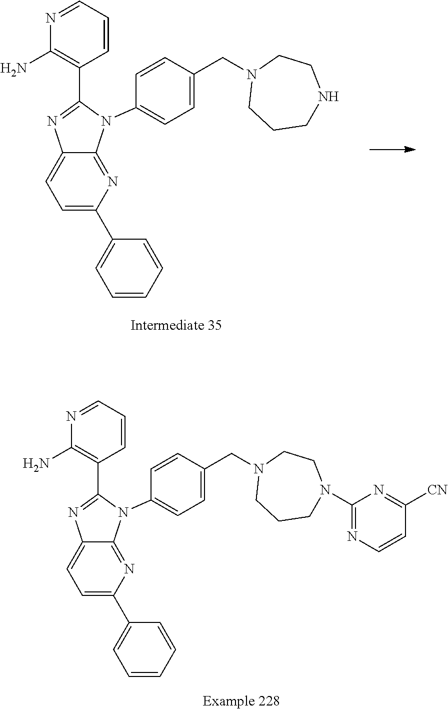 [SMILES: Nc1ncccc1-c1nc2ccc(-c3ccccc3)nc2n1-c1ccc(CN2CCCNCC2)cc1 ; N#Cc1ccnc(N2CCCN(Cc3ccc(-n4c(-c5cccnc5N)nc5ccc(-c6ccccc6)nc54)cc3)CC2)n1]

A mixture of Intermediate 35 (80 mg, 168 μmol), 2-chloropyrimidine-4-carbonitrile (23.5 mg, 168 μmol) and DIEA (109 mg, 841 μmol) in NMP (3 mL) was taken up into a microwave tube. The sealed tube was heated at 130° C. for 1 hr under microwave. The reaction mixture was quenched with H₂O (5 mL) at 0° C., and then diluted with CH₂Cl₂ (10 mL) and extracted with CH₂Cl₂ (15 mL×2). The combined organic layers were washed with brine (15 mL), dried over Na₂SO₄, filtered and concentrated under reduced pressure. After purified by prep-HPLC (column: Waters xbridge 150*25 mm*10 μm; mobile phase: [water (NH₄HCO₃)-ACN]; B %: 54%-84%, 8 min), 2-(4-(4-(2-(2-aminopyridin-3-yl)-5-phenyl-3H-imidazo[4,5-b]pyridin-3-yl)benzyl)-1,4-diazepan-1-yl)pyrimidine-4-carbonitrile (Example 228, 2.4 mg, yield. 2.3%) was obtained as a light-yellow solid. MS: m/z=579.3 [M+H]⁺. ¹H NMR (400 MHz, Dimethylsulfoxide-d₆) δ 8.74-8.56 (m, 1H), 8.27 (d, J=8.4 Hz, 1H), 8.04-7.97 (m, 4H), 7.50-7.43 (m, 7H), 7.15-7.11 (m, 2H), 7.04 (br s, 2H), 6.37-6.32 (m, 1H), 3.87-3.77 (m, 4H), 3.74 (s, 2H), 2.81-2.70 (m, 4H), 1.90-1.84 (m, 2H).

### Example 229: N-(3-(4-(2-(2-aminopyridin-3-yl)-5-phenyl-3H-imidazo[4,5-b]pyridin-3-yl)benzyl)-3-azabicyclo[3.2.1]octan-8-yl)-2-cyanopyrimidine-4-carboxamide

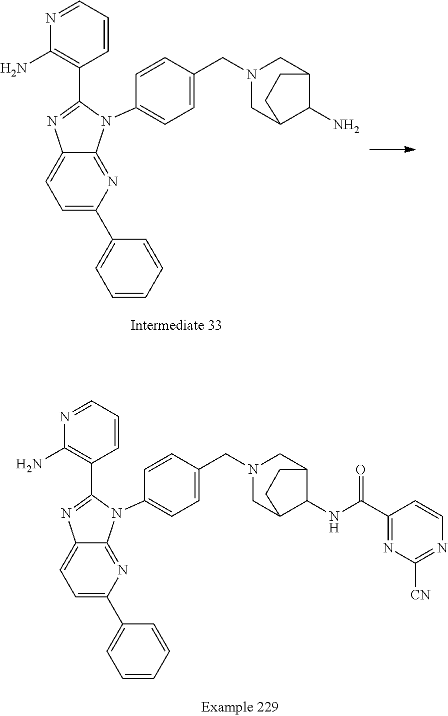 [SMILES: Nc1ncccc1-c1nc2ccc(-c3ccccc3)nc2n1-c1ccc(CN2CC3CCC(C2)C3N)cc1 ; N#Cc1nccc(C(=O)NC2C3CCC2CN(Cc2ccc(-n4c(-c5cccnc5N)nc5ccc(-c6ccccc6)nc54)cc2)C3)n1]

To a solution of Intermediate 33 (100 mg, 199 μmol), 2-cyanopyrimidine-4-carboxylic acid (44.6 mg, 299 μmol), EDCI (57.3 mg, 299 μmol) and HOBt (40.4 mg, 299 μmol) in CH₂Cl₂ (2 mL) was added DIEA (103 mg, 797 μmol). The mixture was stirred at 25° C. for 16 hr. The reaction mixture was diluted with H₂O (10 mL) and extracted with CH₂Cl₂ (20 mL×2). The combined organic layers were washed with brine (10 mL), dried over Na₂SO₄, filtered and concentrated under reduced pressure. The crude product was purified by prep-HPLC (column: Welch Xtimate C₁₈ 150×25 mm×5 μm; mobile phase: [water (HCl)-ACN]; B %: 7%-37%, 10 min) to give N-(3-(4-(2-(2-aminopyridin-3-yl)-5-phenyl-3H-imidazo[4,5-b]pyridin-3-yl)benzyl)-3-azabicyclo[3.2.1]octan-8-yl)-2-cyanopyrimidine-4-carboxamide (Example 229, 12.4 mg, yield: 9.2%, HCl salt) as a yellow solid. MS: m/z=633.3 [M+H]⁺. ¹H NMR (400 MHz, Methanol-d₄) δ 9.17 (d, J=4.8 Hz, 1H), 8.72-8.60 (m, 1H), 8.34-8.25 (m, 2H), 8.04-8.01 (m, 3H), 7.92 (d, J=7.6 Hz, 2H), 7.85 (d, J=7.2 Hz, 1H), 7.73 (d, J=7.6 Hz, 2H), 7.48-7.40 (m, 3H), 6.90-6.87 (m, 1H), 4.52 (s, 2H), 4.21-4.16 (m, 1H), 3.55-3.43 (m, 4H), 2.81-2.73 (m, 2H), 2.23-2.14 (m, 2H), 2.05-2.00 (m, 2H).

### Example 230: N-(3-(4-(2-(2-aminopyridin-3-yl)-5-phenyl-3H-imidazo[4,5-b]pyridin-3-yl)benzyl)-3-azabicyclo[3.2.1]octan-8-yl)-6-cyanopyrimidine-4-carboxamide

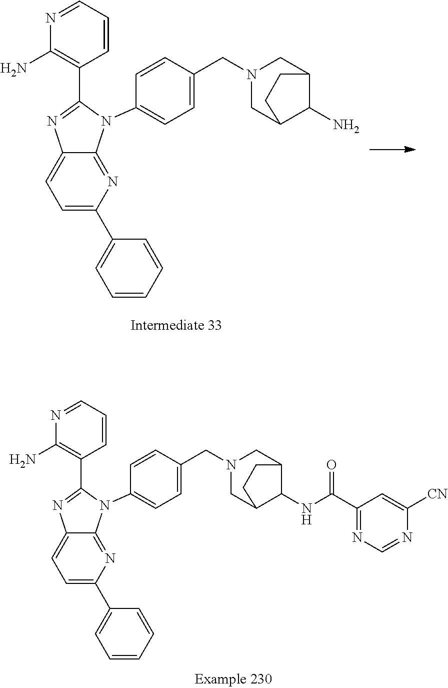 [SMILES: Nc1ncccc1-c1nc2ccc(-c3ccccc3)nc2n1-c1ccc(CN2CC3CCC(C2)C3N)cc1 ; N#Cc1cc(C(=O)NC2C3CCC2CN(Cc2ccc(-n4c(-c5cccnc5N)nc5ccc(-c6ccccc6)nc54)cc2)C3)ncn1]

To a solution of Intermediate 33 (100 mg, 199 μmol), 6-cyanopyrimidine-4-carboxylic acid (38.6 mg, 259 μmol), EDCI (57.3 mg, 299 μmol) and HOBt (40.4 mg, 299 μmol) in CH₂Cl₂ (2 mL) was added DIEA (103 mg, 797 μmol). The mixture was stirred at 25° C. for 16 hr. The reaction mixture was diluted with H₂O (10 mL) and extracted with CH₂Cl₂ (20 mL×2). The combined organic layers were washed with brine (10 mL), dried over Na₂SO₄, filtered, and concentrated under reduced pressure. The crude product was purified by prep-HPLC (column: Welch Xtimate C₁₈ 150×25 mm×5 μm; mobile phase: [water (HCl)-ACN]; B %: 7%-37%, 10 min) to give N-(3-(4-(2-(2-aminopyridin-3-yl)-5-phenyl-3H-imidazo[4,5-b]pyridin-3-yl)benzyl)-3-azabicyclo[3.2.1]octan-8-yl)-6-cyanopyrimidine-4-carboxamide (Example 230, 11.7 mg, yield: 8.2%, HCl salt) as a yellow solid. MS: m/z=633.3 [M+H]⁺. ¹H NMR (400 MHz, Methanol-d₄) δ 9.16 (s, 1H), 8.33 (d, J=8.8 Hz, 1H), 8.17 (d, J=1.2 Hz, 1H), 8.08-8.03 (m, 2H), 7.97-7.94 (m, 2H), 7.91-7.87 (m, 2H), 7.85 (dd, J=7.6, 1.6 Hz, 1H), 7.74 (d, J=8.4 Hz, 2H), 7.44-7.40 (m, 3H), 6.88-6.81 (m, 1H), 4.51 (s, 2H), 3.85 (t, J=4.4 Hz, 1H), 3.52-3.47 (m, 2H), 3.38-3.34 (m, 2H), 2.87-2.82 (m, 2H), 2.15-2.09 (m, 2H), 2.06-1.98 (m, 2H).

### Example 231: 2-((3-(4-(2-(2-Aminopyridin-3-yl)-5-phenyl-3H-imidazo[4,5-b]pyridin-3-yl)benzyl)-3-azabicyclo[3.2.1]octan-8-yl)amino)pyrimidine-4-carbonitrile

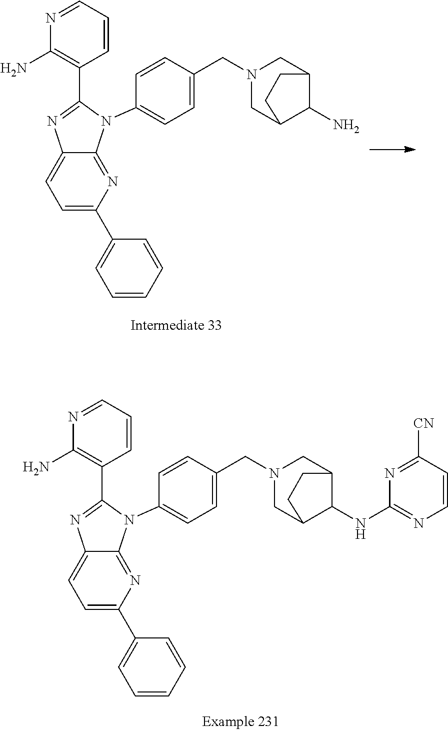 [SMILES: Nc1ncccc1-c1nc2ccc(-c3ccccc3)nc2n1-c1ccc(CN2CC3CCC(C2)C3N)cc1 ; N#Cc1ccnc(NC2C3CCC2CN(Cc2ccc(-n4c(-c5cccnc5N)nc5ccc(-c6ccccc6)nc54)cc2)C3)n1]

A mixture of Intermediate 33 (100 mg, 199 μmol), 2-chloropyrimidine-4-carbonitrile (27.8 mg, 199 μmol) and DIEA (129 mg, 997 μmol) in NMP (3 mL) was taken up into a microwave tube. The sealed tube was heated at 160° C. for 1 hr under microwave. After purified by prep-HPLC (column: Welch Xtimate C₁₈ 150×25 mm×5 μm; mobile phase: [water (NH₃H₂O)-ACN]; B %: 60%-90%, 20 min), 2-((3-(4-(2-(2-aminopyridin-3-yl)-5-phenyl-3H-imidazo[4,5-b]pyridin-3-yl)benzyl)-3-azabicyclo[3.2.1]octan-8-yl)amino)pyrimidine-4-carbonitrile (Example 231, 31.1 mg, yield: 25%) was obtained as a yellow solid. MS: m/z=605.3 [M+H]⁺. ¹H NMR (400 MHz, Dimethylsulfoxide-d₆) δ 8.61 (d, J=4.8 Hz, 1H), 8.27 (d, J=8.4 Hz, 1H), 8.05-7.97 (m, 4H), 7.95-7.71 (m, 1H), 7.52-7.44 (m, 6H), 7.42-7.36 (m, 1H), 7.17-7.04 (m, 4H), 6.40-6.34 (m, 1H), 3.62-3.58 (m, 2H), 2.75-2.56 (m, 4H), 2.46-2.40 (m, 1H), 2.29-2.24 (m, 2H), 1.86-1.63 (m, 4H).

### Example 232: 4-((1-(4-(2-(2-Aminopyridin-3-yl)-5-phenyl-3H-imidazo[4,5-b]pyridin-3-yl)benzyl)piperidin-4-yl)(methyl-d₃)amino)pyrimidine-2-carbonitrile

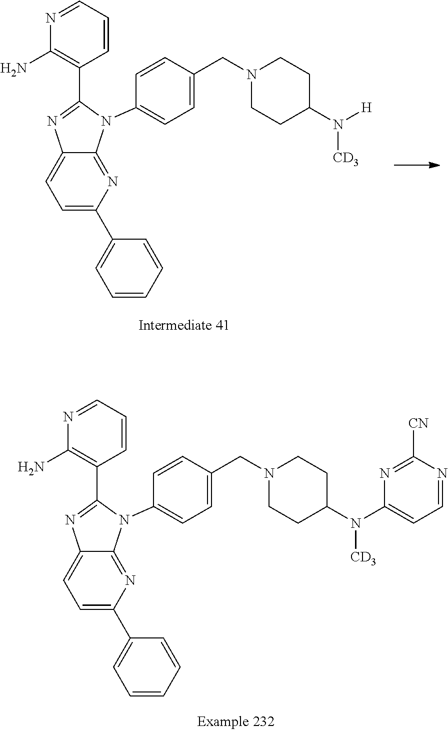 [SMILES: [2H]C([2H])([2H])NC1CCN(Cc2ccc(-n3c(-c4cccnc4N)nc4ccc(-c5ccccc5)nc43)cc2)CC1 ; [2H]C([2H])([2H])N(c1ccnc(C#N)n1)C1CCN(Cc2ccc(-n3c(-c4cccnc4N)nc4ccc(-c5ccccc5)nc43)cc2)CC1]

To a solution of Intermediate 41 (100 mg, 203 μmol) in NMP (2 mL) were added 4-chloropyrimidine-2-carbonitrile (28.3 mg, 203 μmol) and DIEA (78.7 mg, 609 μmol). The mixture was stirred at 130° C. for 0.5 hr under microwave. The mixture was filtered and the filtrate was purified byprep-HPLC (column: Waters xbridge 150×25 mm 10 μm; mobile phase: [water (NH₄HCO₃)-ACN]; B %: 46%-76%, 15 min) to give 4-((1-(4-(2-(2-aminopyridin-3-yl)-5-phenyl-3H-imidazo[4,5-b]pyridin-3-yl)benzyl)piperidin-4-yl)(methyl-d₃)amino)pyrimidine-2-carbonitrile (Example 232, 23.7 mg, yield: 20%) as a light-yellow lyophilized powder. MS: m/z=596.2 [M+H]⁺. ¹H NMR (400 MHz, Methanol-d₄) δ 8.19 (d, J=8.4 Hz, 1H), 8.16 (d, J=6.4 Hz, 1H), 8.03 (d, J=7.2 Hz, 2H), 7.99 (dd, 1=4.8, 1.6 Hz, 1H), 7.94 (d, J=8.4 Hz, 1H), 7.58-7.54 (m, 2H), 7.47 (d, J=8.4 Hz, 2H), 7.45-7.36 (m, 3H), 7.33 (dd, J=7.6, 1.6 Hz, 1H), 6.93-6.71 (m, 1H), 6.48 (dd, J=7.6, 5.2 Hz, 1H), 3.71 (s, 2H), 3.40-3.35 (m, 1H), 3.10 (d, J=12.0 Hz, 2H), 2.35-2.24 (m, 2H), 2.00-1.90 (m, 2H), 1.78-1.67 (m, 2H).

### Example 233: 4-((1-(4-(2-(2-Aminopyridin-3-yl)-5-phenyl-3H-imidazo[4,5-b]pyridin-3-yl)benzyl)piperidin-4-yl)(methyl-d₃)amino)-1,3,5-triazine-2-carbonitrile

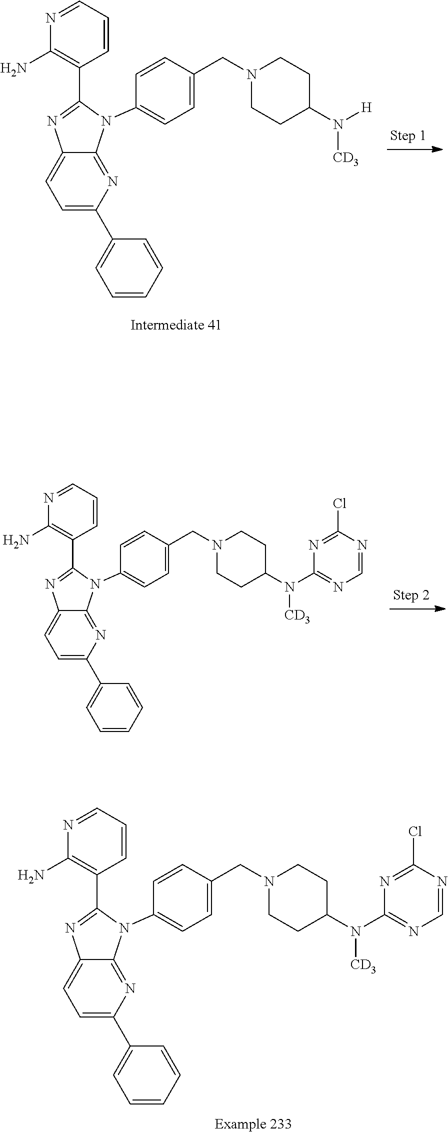 [SMILES: [2H]C([2H])([2H])NC1CCN(Cc2ccc(-n3c(-c4cccnc4N)nc4ccc(-c5ccccc5)nc43)cc2)CC1 ; [2H]C([2H])([2H])N(c1ncnc(Cl)n1)C1CCN(Cc2ccc(-n3c(-c4cccnc4N)nc4ccc(-c5ccccc5)nc43)cc2)CC1 ; [2H]C([2H])([2H])N(c1ncnc(Cl)n1)C1CCN(Cc2ccc(-n3c(-c4cccnc4N)nc4ccc(-c5ccccc5)nc43)cc2)CC1]

### Step 1: N-(1-(4-(2-(2-Aminopyridin-3-yl)-5-phenyl-3H-imidazo[4,5-b]pyridin-3-yl)benzyl)piperidin-4-yl)-4-chloro-N-(methyl-d₃)-1,3,5-triazin-2-amine

To a solution of Intermediate 41 (100 mg, 203 μmol) in THF (8 mL) was added DIEA (78.7 mg, 609 μmol), and then the 2,4-dichloro-1,3,5-triazine (36.5 mg, 244 μmol) in THF (2 mL) was added into the mixture at 0° C. The mixture was stirred at 0° C. for 1 hr. The mixture was concentrated under reduced pressure. The residue was purified by silica gel flash chromatography (Eluent of 0˜5% MeOH in CH₂Cl₂) to give N-(1-(4-(2-(2-Aminopyridin-3-yl)-5-phenyl-3H-imidazo[4,5-b]pyridin-3-yl)benzyl)piperidin-4-yl)-4-chloro-N-(methyl-d₃)-1,3,5-triazin-2-amine (50 mg crude, yield: 44%) as a light-yellow solid. MS: m/z=606.3 [M+H]⁺. ¹H NMR (400 MHz, Methanol-d₄) δ 8.35 (d, J=6.4 Hz, 1H), 8.20 (d, J=8.4 Hz, 1H), 8.05-8.02 (m, 2H), 7.98 (dd, J=4.8, 1.6 Hz, 1H), 7.94 (d, J=8.4 Hz, 1H), 7.56-7.54 (m, 2H), 7.48-7.32 (m, 6H), 6.51-6.46 (m, 1H), 3.68 (d, J=6.4 Hz, 1H), 3.35 (s, 2H), 3.12-3.05 (m, 2H), 2.30-2.20 (m, 2H), 1.99-1.90 (m, 2H), 1.75-1.70 (m, 2H).

### Step 2: 4-((1-(4-(2-(2-Aminopyridin-3-yl)-5-phenyl-3H-imidazo[4,5-b]pyridin-3-yl)benzyl)piperidin-4-yl)(methyl-d₃)amino)-1,3,5-triazine-2-carbonitrile

To a solution of N-(1-(4-(2-(2-aminopyridin-3-yl)-5-phenyl-3H-imidazo[4,5-b]pyridin-3-yl)benzyl)piperidin-4-yl)-4-chloro-N-(methyl-d₃)-1,3,5-triazin-2-amine (50 mg, 82.5 μmol) in DMSO (1 mL) were added DABCO (1.85 mg, 16.5 μmol) and KCN (10 mg, 156 μmol). The mixture was stirred at 0° C. for 1 hr. The reaction mixture was poured into water (10 mL). The resulting mixture was extracted with EtOAc (10 mL×2). The combined organic layers were washed with brine (10 mL), dried over anhydrous Na₂SO₄, filtered and concentrated. The residue was purified by silica gel flash chromatography (Eluent of 0˜7% MeOH in CH₂Cl₂) to give 4-((1-(4-(2-(2-Aminopyridin-3-yl)-5-phenyl-3H-imidazo[4,5-b]pyridin-3-yl)benzyl)piperidin-4-yl)(methyl-d₃)amino)-1,3,5-triazine-2-carbonitrile (Example 233, 18.4 mg, yield: 34%) as a light-yellow solid. MS: m/z=597.4 [M+H]⁺. ¹H NMR (400 MHz, Methanol-d₄) δ 8.58 (d, J=6.8 Hz, 1H), 8.20 (d, J=8.4 Hz, 1H), 8.04 (d, J=7.2 Hz, 2H), 8.01-7.97 (m, 1H), 7.95 (d, J=8.4 Hz, 1H), 7.58-7.53 (m, 2H), 7.49-7.41 (m, 4H), 7.38-7.32 (m, 2H), 6.54-6.43 (m, 1H), 3.68 (d, J=7.6 Hz, 2H), 3.52-3.40 (m, 1H), 3.11-3.06 (m, 2H), 2.28-2.19 (m, 2H), 2.00-1.97 (m, 2H), 1.75-1.69 (m, 2H).

### Example 234: N-(1-(4-(2-(2-Aminopyridin-3-yl)-5-phenyl-3H-imidazo[4,5-b]pyridin-3-yl)benzyl)piperidin-4-yl)-2-cyano-N-(methyl-d₃)pyrimidine-4-carboxamide

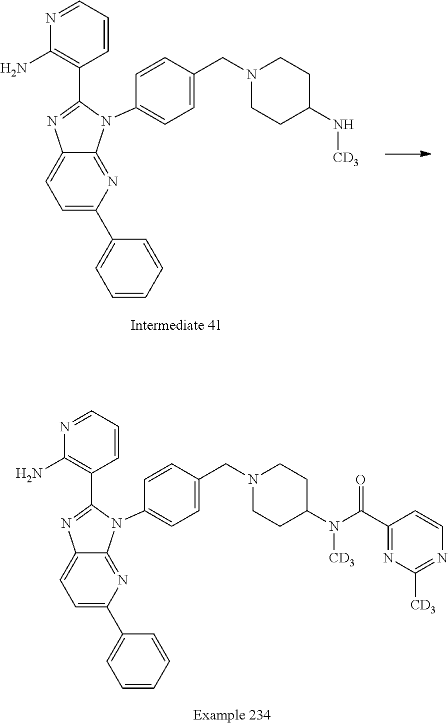 [SMILES: [2H]C([2H])([2H])NC1CCN(Cc2ccc(-n3c(-c4cccnc4N)nc4ccc(-c5ccccc5)nc43)cc2)CC1 ; [2H]C([2H])([2H])c1nccc(C(=O)N(C2CCN(Cc3ccc(-n4c(-c5cccnc5N)nc5ccc(-c6ccccc6)nc54)cc3)CC2)C([2H])([2H])[2H])n1]

To solution of Intermediate 41 (50 mg, 102 μmol), 2-cyanopyrimidine-4-carboxylic acid (19.7 mg, 132 μmol), EDCI (29.2 mg, 152 μmol) and HOBt (20.6 mg, 152 μmol) in CH₂Cl₂ (2 mL) was added DIEA (52.5 mg, 406 μmol). The mixture was stirred at 25° C. for 16 hr. The reaction mixture was diluted with H₂O (10 mL) and extracted with CH₂Cl₂ (20 mL×2). The combined organic layers were washed with brine (10 mL), dried over Na₂SO₄, filtered, and concentrated under reduced pressure. The crude product was purified by prep-TLC (CH₂Cl₂:MeOH=20:1) to give N-(1-(4-(2-(2-aminopyridin-3-yl)-5-phenyl-3H-imidazo[4,5-b]pyridin-3-yl)benzyl)piperidin-4-yl)-2-cyano-N-(methyl-d₃)pyrimidine-4-carboxamide (Example 234, 15.5 mg, yield: 24%) as a yellow solid. MS: m/z=624.4 [M+H]⁺. ¹H NMR (400 MHz, Dimethylsulfoxide-d₆) δ 9.23-9.03 (m, 1H), 8.28 (d, J=7.6 Hz, 1H), 8.06-7.97 (m, 5H), 7.54-7.39 (m, 7H), 7.19-7.13 (m, 1H), 7.06-6.96 (m, 2H), 6.43-6.34 (m, 1H), 3.66-3.39 (m, 2H), 3.14-2.96 (m, 1H), 2.94-2.74 (m, 1H), 2.21-2.12 (m, 1H), 1.93-1.85 (m, 2H), 1.73-1.60 (m, 2H), 1.30-1.19 (m, 2H).

### Example 235: 4-(4-(4-(2-(2-Aminopyridin-3-yl)-5-morpholino-3H-imidazo[4,5-b]pyridin-3-yl)benzyl)piperazin-1-yl)pyrimidine-2-carbonitrile

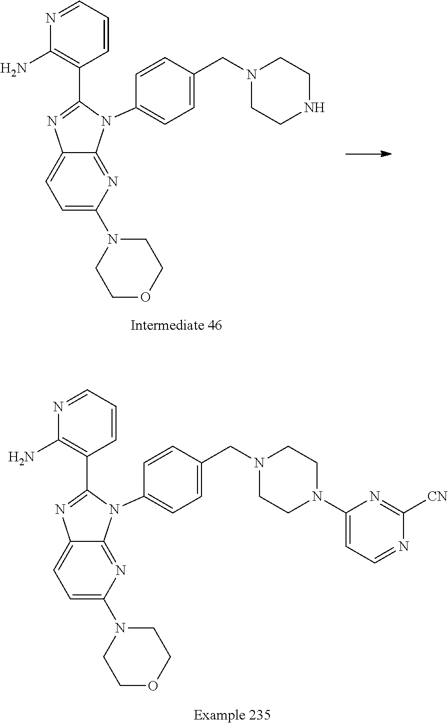 [SMILES: Nc1ncccc1-c1nc2ccc(N3CCOCC3)nc2n1-c1ccc(CN2CCNCC2)cc1 ; N#Cc1nccc(N2CCN(Cc3ccc(-n4c(-c5cccnc5N)nc5ccc(N6CCOCC6)nc54)cc3)CC2)n1]

A mixture of Intermediate 46 (100 mg, 213 μmol), 4-chloropyrimidine-2-carbonitrile (29.7 mg, 213 μmol) and DIEA (110 mg, 850 μmol) in NMP (2 mL) was taken up into a microwave tube. The sealed tube was heated at 130° C. for 1 hr under microwave. The reaction mixture was quenched with H₂O (5 mL) at 0° C., and then diluted with CH₂Cl₂ (10 mL) and extracted with CH₂Cl₂ (15 mL×2). The combined organic layers were washed with brine (15 mL), dried over Na₂SO₄, filtered, and concentrated under reduced pressure. After purified by prep-HPLC (column: Phenomenex C18 150*25 mm*5 μm; mobile phase: [water (NH₄HCO₃)-ACN]; B %: 30%-60%, over 14 min), 4-(4-(4-(2-(2-aminopyridin-3-yl)-5-morpholino-3H-imidazo[4,5-b]pyridin-3-yl)benzyl)piperazin-1-yl)pyrimidine-2-carbonitrile (Example 235, 15.9 mg, yield: 13%) was obtained as a brown lyophilized powder. MS: m/z=574.3 [M+H]⁺. ¹H NMR (400 MHz, Dimethylsulfoxide-d₆) δ 8.27 (d, J=6.4 Hz, 1H), 7.98 (d, J=8.8 Hz, 1H), 7.93 (dd, J=4.8, 2.0 Hz, 1H), 7.46 (d, J=8.4 Hz, 2H), 7.33 (d, J=8.4 Hz, 2H), 7.12 (d, J=6.4 Hz, 1H), 7.04 (br s, 2H), 6.98 (dd, J=8.0, 2.0 Hz, 1H), 6.90 (d, J=9.2 Hz, 1H), 6.30 (dd, J=7.6, 4.8 Hz, 1H), 3.72-3.64 (m, 8H), 3.62 (s, 2H), 3.42-3.38 (m, 4H), 2.49-2.45 (m, 4H).

### Example 236: 2-(4-(4-(2-(2-Aminopyridin-3-yl)-5-morpholino-3H-imidazo[4,5-b]pyridin-3-yl)benzyl)piperazin-1-yl)pyrimidine-4-carbonitrile

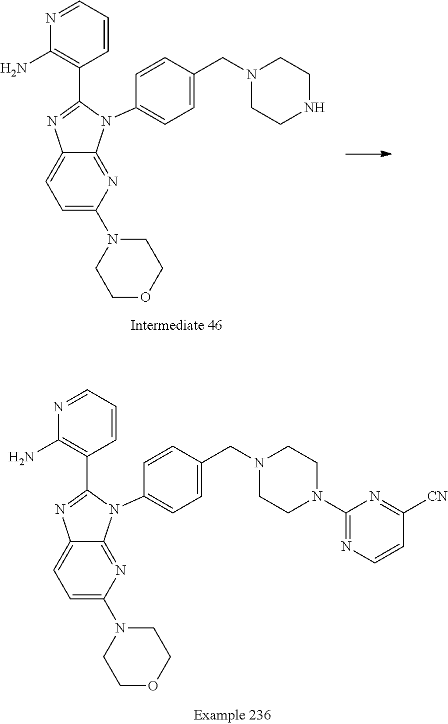 [SMILES: Nc1ncccc1-c1nc2ccc(N3CCOCC3)nc2n1-c1ccc(CN2CCNCC2)cc1 ; N#Cc1ccnc(N2CCN(Cc3ccc(-n4c(-c5cccnc5N)nc5ccc(N6CCOCC6)nc54)cc3)CC2)n1]

A mixture of Intermediate 46 (200 mg, 425 μmol), 2-chloropyrimidine-4-carbonitrile (59.3 mg, 425 μmol) and DIEA (220 mg, 1.7 mmol) in NMP (2 mL) was taken up into a microwave tube. The sealed tube was heated at 130° C. for 1 hr under microwave. The reaction mixture was quenched with H₂O (5 mL) at 0° C., and then diluted with CH₂Cl₂ (10 mL) and extracted with CH₂Cl₂ (15 mL×2). The combined organic layers were washed with brine (15 mL), dried over Na₂SO₄, filtered and concentrated under reduced pressure. After purified by prep-HPLC (column: Phenomenex C18 150*25 mm*5 μm; mobile phase: [water (NH₄HCO₃)-ACN]; B %: 41%-71%, over 14 min), 2-(4-(4-(2-(2-aminopyridin-3-yl)-5-morpholino-3H-imidazo[4,5-b]pyridin-3-yl)benzyl)piperazin-1-yl)pyrimidine-4-carbonitrile (Example 236, 52.9 mg, yield: 22%) was obtained as a brown lyophilized powder. MS: m/z=574.3 [M+H]⁺. ¹H NMR (400 MHz, Dimethylsulfoxide-d₆) δ 8.65 (d, J=4.8 Hz, 1H), 7.99 (d, J=8.8 Hz, 1H), 7.93 (dd, J=4.8, 1.6 Hz, 1H), 7.47 (d, J=8.4 Hz, 2H), 7.34 (d, J=8.4 Hz, 2H), 7.16 (d, J=4.8 Hz, 1H), 7.03 (br s, 2H), 6.99 (dd, J=8.0, 2.0 Hz, 1H), 6.90 (d, J=9.2 Hz, 1H), 6.31 (dd, J=8.0, 5.2 Hz, 1H), 3.82-3.76 (m, 4H), 3.71-3.66 (m, 4H), 3.62 (s, 2H), 3.42-3.38 (m, 4H), 2.49-2.44 (m, 4H).

### Example 237: 6-(4-(4-(2-(2-Aminopyridin-3-yl)-5-morpholino-3H-imidazo[4,5-b]pyridin-3-yl)benzyl)piperazin-1-yl)pyrimidine-4-carbonitrile

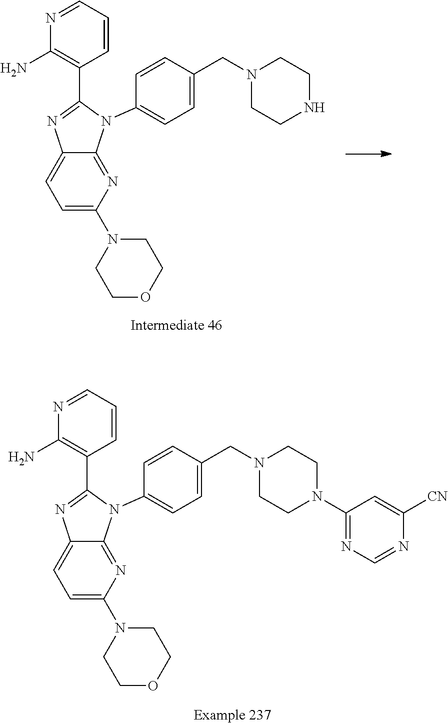 [SMILES: Nc1ncccc1-c1nc2ccc(N3CCOCC3)nc2n1-c1ccc(CN2CCNCC2)cc1 ; N#Cc1cc(N2CCN(Cc3ccc(-n4c(-c5cccnc5N)nc5ccc(N6CCOCC6)nc54)cc3)CC2)ncn1]

A mixture of Intermediate 46 (200 mg, 425 μmol), 6-chloropyrimidine-4-carbonitrile (59.3 mg, 425 μmol) and DIEA (220 mg, 1.7 mmol) in NMP (2 mL) was taken up into a microwave tube The sealed tube was heated at 130° C. for 1 hr under microwave. The reaction mixture was quenched with H₂O (5 mL) at 0° C., and then diluted with CH₂Cl₂ (10 mL) and extracted with CH₂Cl₂ (15 mL×2). The combined organic layers were washed with brine (15 mL), dried over Na₂SO₄, filtered and concentrated under reduced pressure. After purified by prep-HPLC (column: Phenomenex C18 150*25 mm*5 μm; mobile phase: [water (NH₄HCO₃)-ACN]; B %: 40%-70%, over 10 min), 6-(4-(4-(2-(2-aminopyridin-3-yl)-5-morpholino-3H-imidazo[4,5-b]pyridin-3-yl)benzyl)piperazin-1-yl)pyrimidine-4-carbonitrile (Example 237, 42.2 mg, yield: 17%) was obtained as a brown lyophilized powder. MS: m/z=574.2 [M+H]⁺. ¹H NMR (400 MHz, Dimethylsulfoxide-d₆) δ 8.57 (s, 1H), 7.98 (d, J=8.8 Hz, 1H), 7.93 (dd, J=4.8, 1.6 Hz, 1H), 7.57 (s, 1H), 7.46 (d, J=8.4 Hz, 2H), 7.33 (d, J=8.4 Hz, 2H), 7.03 (br s, 2H), 6.98 (dd, J=7.6, 1.6 Hz, 1H), 6.90 (d, J=8.8 Hz, 1H), 6.28 (dd, J=7.6, 4.8 Hz, 1H), 3.81-3.65 (m, 8H), 3.61 (s, 2H), 3.42-3.38 (m, 4H), 2.49-2.44 (m, 4H).

### Example 238: 4-(4-(4-(2-(2-Aminopyridin-3-yl)-5-(6-oxo-1,6-dihydropyridin-2-yl)-3H-imidazo[4,5-b]pyridin-3-yl)benzyl)piperazin-1-yl)pyrimidine-2-carbonitrile

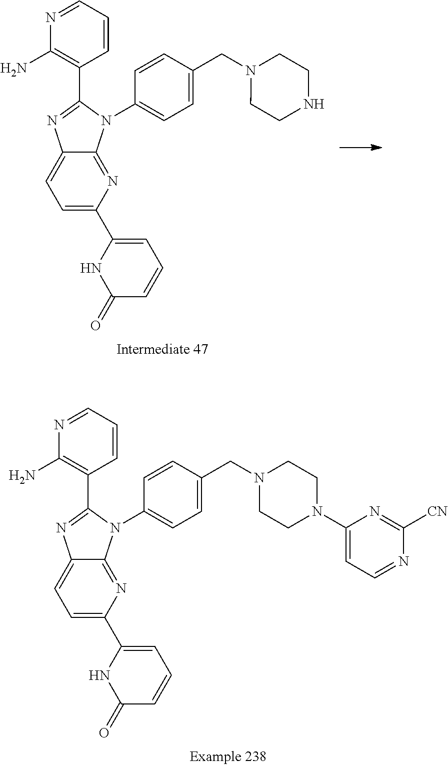 [SMILES: Nc1ncccc1-c1nc2ccc(-c3cccc(=O)[nH]3)nc2n1-c1ccc(CN2CCNCC2)cc1 ; N#Cc1nccc(N2CCN(Cc3ccc(-n4c(-c5cccnc5N)nc5ccc(-c6cccc(=O)[nH]6)nc54)cc3)CC2)n1]

A mixture of Intermediate 47 (100 mg, 209 μmol), 4-chloropyrimidine-2-carbonitrile (35 mg, 251 μmol) and DIEA (108 mg, 836 μmol) in NMP (2 mL) was taken up into a microwave tube. The sealed tube was heated at 130° C. for 1 hr under microwave. The reaction mixture was quenched with H₂O (5 mL) at 0° C., and then diluted with CH₂Cl₂ (10 mL) and extracted with CH₂Cl₂ (15 mL×2). The combined organic layers were washed with brine (15 mL), dried over Na₂SO₄, filtered and concentrated under reduced pressure. After purified by prep-HPLC (column: Phenomenex C18 150*25 mm*10 μm; mobile phase: [water (NH₄HCO₃)-ACN]; B %: 20%-50%, over 14 min), 4-(4-(4-(2-(2-aminopyridin-3-yl)-5-(6-oxo-1,6-dihydropyridin-2-yl)-3H-imidazo[4,5-b]pyridin-3-yl)benzyl)piperazin-1-yl)pyrimidine-2-carbonitrile (Example 238, 19.3 mg, yield: 16%) was obtained as a brown lyophilized powder. MS: m/z=582.2 [M+H]⁺. ¹H NMR (400 MHz, Dimethylsulfoxide-d₆) δ 10.80 (br s, 1H), 8.34 (d, J=8.0 Hz, 1H), 8.27 (d, J=6.4 Hz, 1H), 8.21-8.11 (m, 1H), 8.01 (dd, J=4.8, 1.6 Hz, 1H), 7.67-7.45 (m, 6H), 7.17-7.15 (m, 1H), 7.12 (d, J=6.4 Hz, 1H), 7.03 (br s, 2H), 6.52-6.42 (m, 1H), 6.38 (dd, J=7.6, 4.8 Hz, 1H), 3.79-3.67 (m, 4H), 3.66 (s, 2H), 2.60-2.52 (m, 4H).

### Example 239: 4-((1-(4-(2-(2-Aminopyridin-3-yl)-5-morpholino-3H-imidazo[4,5-b]pyridin-3-yl)benzyl)piperidin-4-yl)amino)pyrimidine-2-carbonitrile

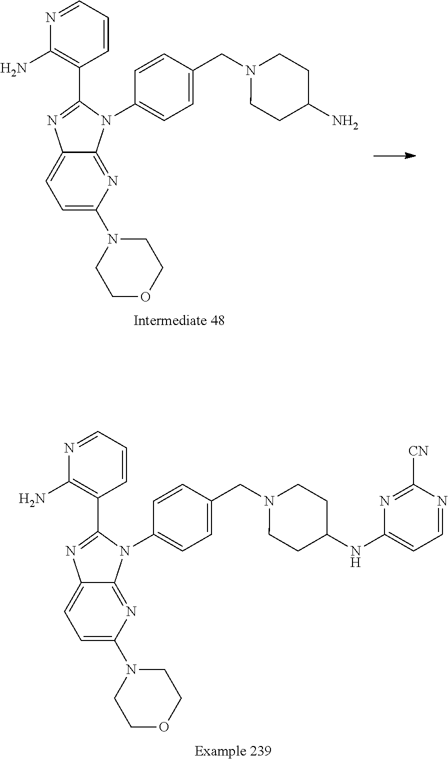 [SMILES: Nc1ncccc1-c1nc2ccc(N3CCOCC3)nc2n1-c1ccc(CN2CCC(N)CC2)cc1 ; N#Cc1nccc(NC2CCN(Cc3ccc(-n4c(-c5cccnc5N)nc5ccc(N6CCOCC6)nc54)cc3)CC2)n1]

A mixture of Intermediate 48 (100 mg, 206 μmol), 4-chloropyrimidine-2-carbonitrile (28.8 mg, 206 μmol) and DIEA (133 mg, 1.03 mmol) in NMP (2 mL) was taken up into a microwave tube. The sealed tube was heated at 130° C. for 30 min under microwave. The reaction mixture was filtered. After purified by prep-HPLC (column: Waters xbridge 150*25 mm 10 μm; mobile phase: [water (NH₄HCO₃)-ACN]; gradient: 28%-58% B over 18 min), 4-((1-(4-(2-(2-aminopyridin-3-yl)-5-morpholino-3H-imidazo[4,5-b]pyridin-3-yl)benzyl)piperidin-4-yl)amino)pyrimidine-2-carbonitrile (Example 239, 39.3 mg, yield: 32%) was obtained as a light-yellow powder. MS: m/z=588.4 [M+H]⁺. ¹H NMR (400 MHz, Dimethylsulfoxide-d₆) δ 8.08 (d, J=6.0 Hz, 1H), 8.04 (d, J=6.4 Hz, 1H), 7.98 (d, J=8.8 Hz, 1H), 7.93 (dd, J=4.4, 1.2 Hz, 1H), 7.42 (d, J=8.0 Hz, 2H), 7.31 (d, J=8.4 Hz, 2H), 7.03 (br s, 2H), 7.00-6.97 (m, 1H), 6.89 (d, J=9.2 Hz, 1H), 6.67 (d, J=6.0 Hz, 1H), 6.30 (dd, J=7.6, 5.2 Hz, 1H), 3.86-3.75 (m, 1H), 3.70-3.65 (m, 4H), 3.56 (s, 2H), 3.45-3.35 (m, 4H), 2.84-2.75 (m, 2H), 2.20-2.15 (m, 2H), 1.90-1.82 (m, 2H), 1.54-1.45 (m, 2H).

### Example 240: 2-((1-(4-(2-(2-Aminopyridin-3-yl)-5-morpholino-3H-imidazo[4,5-b]pyridin-3-yl)benzyl)piperidin-4-yl)amino)pyrimidine-4-carbonitrile

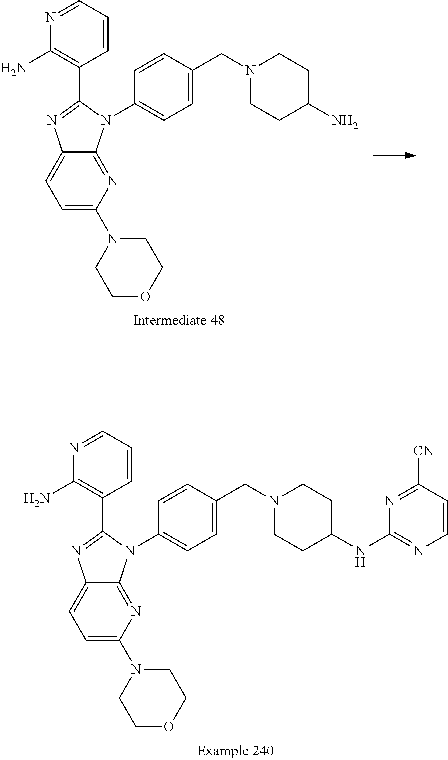 [SMILES: Nc1ncccc1-c1nc2ccc(N3CCOCC3)nc2n1-c1ccc(CN2CCC(N)CC2)cc1 ; N#Cc1ccnc(NC2CCN(Cc3ccc(-n4c(-c5cccnc5N)nc5ccc(N6CCOCC6)nc54)cc3)CC2)n1]

A mixture of Intermediate 48 (200 mg, 413 μmol), 2-chloropyrimidine-4-carbonitrile (57.6 mg, 413 μmol) and DIEA (267 mg, 2.06 mmol) in NMP (3 mL) was taken up into a microwave tube. The sealed tube was heated at 130° C. for 30 min under microwave. The reaction mixture was filtered. After purified by prep-HPLC (column: Phenomenex C18 150*25 mm*10 μm; mobile phase: [water (NH₄HCO₃)-ACN]; gradient: 35%-65% B over 10 min), 2-((1-(4-(2-(2-aminopyridin-3-yl)-5-morpholino-3H-imidazo[4,5-b]pyridin-3-yl)benzyl)piperidin-4-yl)amino)pyrimidine-4-carbonitrile (Example 240, 23.9 mg, 25% yield) was obtained as a yellow powder. MS: m/z=588.3 [M+H]⁺. ¹H NMR (400 MHz, Dimethylsulfoxide-d₆) δ 8.60-8.46 (m, 1H), 7.97 (d, J=9.2 Hz, 1H), 7.93 (dd, J=4.8, 1.6 Hz, 1H), 7.85 (d, J=7.6 Hz, 1H), 7.41 (d, J=8.4 Hz, 2H), 7.30 (d, J=8.4 Hz, 2H), 7.06 (d, J=4.8 Hz, 1H), 7.03 (br s, 2H), 6.99 (dd, J=8.0, 2.0 Hz, 1H), 6.88 (d, J=9.2 Hz, 1H), 6.30 (dd, J=8.0, 5.2 Hz, 1H), 3.68-3.65 (m, 4H), 3.54 (s, 2H), 3.41-3.38 (m, 5H), 2.85-2.75 (m, 2H), 2.10-2.00 (m, 2H), 1.88-1.78 (m, 2H), 1.59-1.48 (m, 2H).

### Example 241: 6-((1-(4-(2-(2-Aminopyridin-3-yl)-5-morpholino-3H-imidazo[4,5-b]pyridin-3-yl)benzyl)piperidin-4-yl)amino)pyrimidine-4-carbonitrile

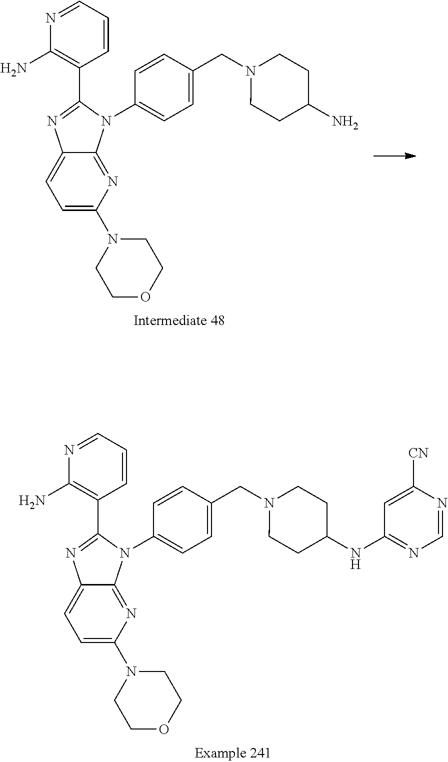 [SMILES: Nc1ncccc1-c1nc2ccc(N3CCOCC3)nc2n1-c1ccc(CN2CCC(N)CC2)cc1 ; N#Cc1cc(NC2CCN(Cc3ccc(-n4c(-c5cccnc5N)nc5ccc(N6CCOCC6)nc54)cc3)CC2)ncn1]

A mixture of Intermediate 48 (200 mg, 413 μmol), 6-chloropyrimidine-4-carbonitrile (57.6 mg, 413 μmol) and DIEA (267 mg, 2.06 mmol) in NMP (2 mL) was taken up into a microwave tube. The sealed tube was heated at 130° C. for 30 min under microwave. The reaction mixture was filtered. After purified by prep-HPLC (column: Waters xbridge 150*25 mm 10 μm; mobile phase: [water (NH₄HCO₃)-ACN]; gradient: 31%-61% B over 10 min), 6-((1-(4-(2-(2-aminopyridin-3-yl)-5-morpholino-3H-imidazo[4,5-b]pyridin-3-yl)benzyl)piperidin-4-yl)amino)pyrimidine-4-carbonitrile (Example 241, 79.9 mg, 31% yield) was obtained as a yellow powder. MS: m/z=588.3 [M+H]⁺. ¹H NMR (400 MHz, Dimethylsulfoxide-d₆) δ 8.49 (s, 1H), 8.07 (d, J=6.8 Hz, 1H), 7.97 (d, J=8.8 Hz, 1H), 7.95-7.89 (m, 1H), 7.42 (d, J=8.4 Hz, 2H), 7.31 (d, J=8.4 Hz, 2H), 7.04 (br s, 2H), 7.00-6.96 (m, 1H), 6.95-6.85 (m, 2H), 6.30 (dd, J=7.6, 4.8 Hz, 1H), 3.90-3.75 (m, 1H), 3.70-3.60 (m, 4H), 3.55 (s, 2H), 3.43-3.35 (m, 4H), 2.85-2.75 (m, 2H), 2.15-2.04 (m, 2H), 1.90-1.75 (m, 2H), 1.55-1.43 (m, 2H).

### Example 242: 4-(4-(4-(2-(2-Aminopyridin-3-yl)-5-(4-fluorophenyl)-3H-imidazo[4,5-b]pyridin-3-yl)benzyl)piperazin-1-yl)pyrimidine-2-carbonitrile

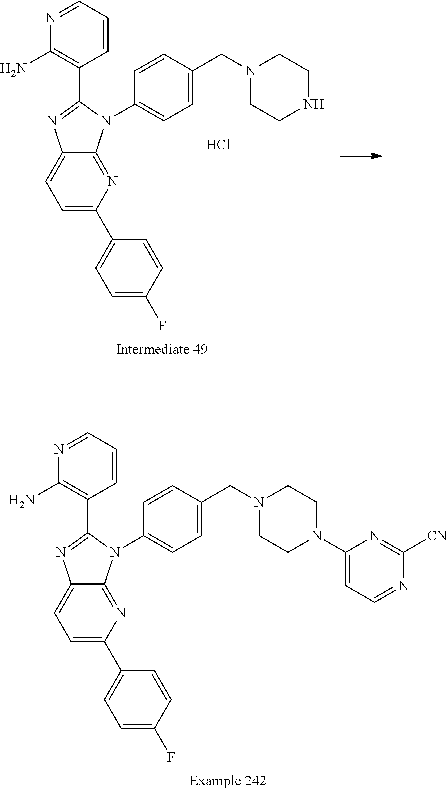 [SMILES: Nc1ncccc1-c1nc2ccc(-c3ccc(F)cc3)nc2n1-c1ccc(CN2CCNCC2)cc1 ; Cl ; N#Cc1nccc(N2CCN(Cc3ccc(-n4c(-c5cccnc5N)nc5ccc(-c6ccc(F)cc6)nc54)cc3)CC2)n1]

A mixture of Intermediate 49 (100 mg, 209 μmol), 4-chloropyrimidine-2-carbonitrile (29.1 mg, 209 μmol) and DIEA (27 mg, 209 μmol) in NMP (2 mL) was taken up into a microwave tube. The sealed tube was heated at 130° C. for 1 hr under microwave. After purified by prep-HPLC (column: Waters xbridge 150×25 mm×10 μm; mobile phase: [water (NH₄HCO₃)-ACN]; B %: 45%-75%, 10 min), 4-(4-(4-(2-(2-aminopyridin-3-yl)-5-(4-fluorophenyl)-3H-imidazo[4,5-b]pyridin-3-yl)benzyl)piperazin-1-yl)pyrimidine-2-carbonitrile (Example 242, 35.2 mg, yield: 29%) was obtained as a yellow solid. MS: m/z=583.3 [M+H]⁺. ¹H NMR (400 MHz, Dimethylsulfoxide-d₆) δ 8.27 (d, J=7.6 Hz, 2H), 8.08 (dd, J=8.4, 6.0 Hz, 2H), 8.01-7.95 (m, 2H), 7.54-7.45 (m, 4H), 7.30 (t, J=8.8 Hz, 2H), 7.16-7.10 (m, 2H), 7.03 (s, 2H), 6.39-6.32 (m, 1H), 3.78-3.65 (m, 4H), 3.65 (s, 2H), 2.52-2.51 (m, 4H). ¹⁹F NMR (400 MHz, Dimethylsulfoxide-d₆) δ −113.56.

### Example 243: 2-(4-(4-(2-(2-Aminopyridin-3-yl)-5-(4-fluorophenyl)-3H-imidazo[4,5-b]pyridin-3-yl)benzyl)piperazin-1-yl)pyrimidine-4-carbonitrile

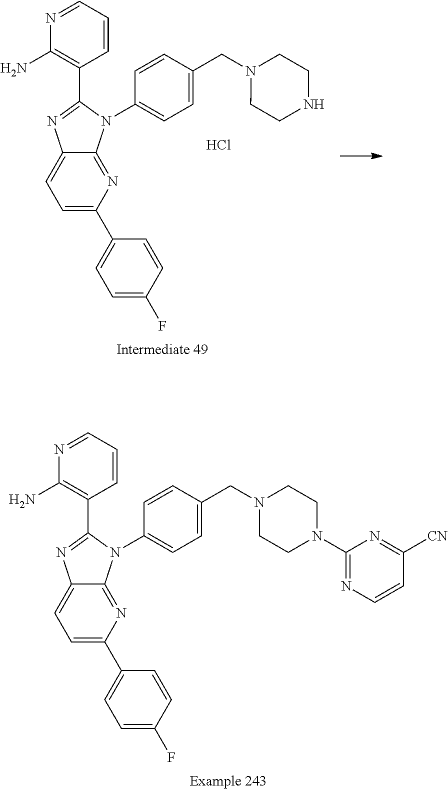 [SMILES: Nc1ncccc1-c1nc2ccc(-c3ccc(F)cc3)nc2n1-c1ccc(CN2CCNCC2)cc1 ; Cl ; N#Cc1ccnc(N2CCN(Cc3ccc(-n4c(-c5cccnc5N)nc5ccc(-c6ccc(F)cc6)nc54)cc3)CC2)n1]

A mixture of Intermediate 49 (80 mg, 167 μmol), 2-chloropyrimidine-4-carbonitrile (23.3 mg, 167 μmol), and DIEA (43.1 mg, 334 μmol) in NMP (2 mL) was taken up into a microwave tube. The sealed tube was heated at 130° C. for 1 hr under microwave. After purified by prep-HPLC (column: Waters xbridge 150×25 mm×10 μm; mobile phase: [water (NH₄HCO₃)-ACN]; B %: 56%-86%, 10 min), 2-(4-(4-(2-(2-aminopyridin-3-yl)-5-(4-fluorophenyl)-3H-imidazo[4,5-b]pyridin-3-yl)benzyl)piperazin-1-yl)pyrimidine-4-carbonitrile (Example 243, 56.3 mg, yield: 58%) was obtained as a brown solid. MS: m/z=583.3 [M+H]⁺. ¹H NMR (400 MHz, Dimethylsulfoxide-d₆) δ 8.65 (d, J=4.8 Hz, 1H), 8.27 (d, J=8.4 Hz, 1H), 8.11-8.06 (m, 2H), 8.01-7.97 (m, 2H), 7.53-7.45 (m, 4H), 7.33-7.28 (m, 2H), 7.17-7.13 (m, 2H), 7.03 (s, 2H), 6.37 (dd, J=8.0, 4.8 Hz, 1H), 3.82-3.77 (m, 4H), 3.64 (s, 2H), 2.51-2.51 (m, 4H). ¹⁹F NMR (400 MHz, Dimethylsulfoxide-d₆) 5-113.56.

### Example 244: N-(4-(2-(2-Aminopyridin-3-yl)-3H-imidazo[4,5-b]pyridin-3-yl)benzyl)-2-cyanothiazole-4-carboxamide

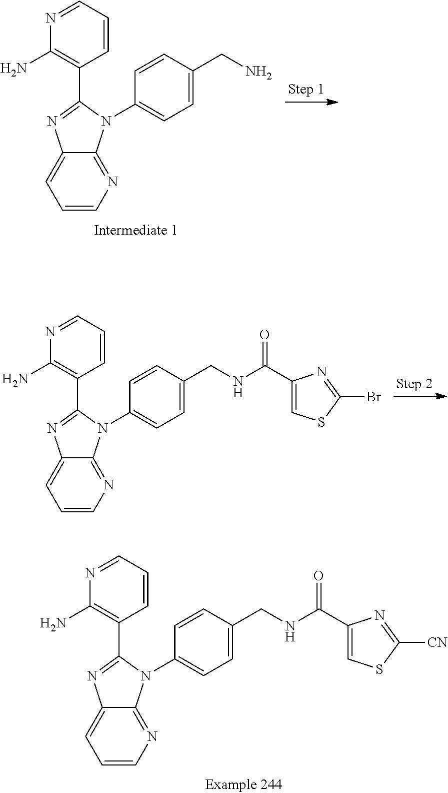 [SMILES: NCc1ccc(-n2c(-c3cccnc3N)nc3cccnc32)cc1 ; Nc1ncccc1-c1nc2cccnc2n1-c1ccc(CNC(=O)c2csc(Br)n2)cc1 ; N#Cc1nc(C(=O)NCc2ccc(-n3c(-c4cccnc4N)nc4cccnc43)cc2)cs1]

### Step 1: N-(4-(2-(2-aminopyridin-3-yl)-3H-imidazo[4,5-b]pyridin-3-yl)benzyl)-2-bromothiazole-4-carboxamide

To a solution of 3-(3-(4-(aminomethyl)phenyl)-3H-imidazo[4,5-b]pyridin-2-yl)pyridin-2-amine (138 mg, 437 μmol) and 2-bromothiazole-4-carboxylic acid (100 mg, 480 μmol) in DMF (2 mL) were added EDCI (125 mg, 655 μmol), HOBt (88 mg, 655 μmol), and DIEA (282 mg, 2.2 mmol). The mixture was stirred at 25° C. for 3 hr. The mixture was quenched with water (20 mL) and extracted with EtOAc (25 mL×3). The combined organic layers were dried over Na₂SO₄, filtered, concentrated and purified by silica gel flash chromatography (eluent of 0-3% MeOH in CH₂Cl₂), N-(4-(2-(2-aminopyridin-3-yl)-3H-imidazo[4,5-b]pyridin-3-yl)benzyl)-2-bromothiazole-4-carboxamide (53 mg, yield: 43%) was obtained as a yellow solid. MS: m/z=506.1, 508.0 [M+H]⁺. ¹H NMR (400 MHz, Dimethylsulfoxide-d₆) δ 9.20 (t, J=6.4 Hz, 1H), 8.35-8.28 (m, 2H), 8.19 (dd, J=8.0, 1.2 Hz, 1H), 7.99 (dd, J=4.8, 1.6 Hz, 1H), 7.47-7.41 (m, 2H), 7.41-7.35 (m, 3H), 7.23 (dd, J=7.6, 1.6 Hz, 1H), 6.97 (br s, 2H), 6.41 (dd, J=7.6, 4.8 Hz, 1H), 4.51 (d, J=6.4 Hz, 2H).

### Step 2: N-(4-(2-(2-aminopyridin-3-yl)-3H-imidazo[4,5-b]pyridin-3-yl)benzyl)-2-cyanothiazole-4-carboxamide

To a solution of N-[[4-[2-(2-amino-3-pyridyl)imidazo[4,5-b]pyridin-3-yl]phenyl]methyl]-2-bromo-thiazole-4-carboxamide (100 mg, 198 μmol) in pyridine (3.5 mL) was added CuCN (37.1 mg, 415 μmol) at 25° C. The mixture was stirred at 145° C. for 6 hr. The mixture was filtered and concentrated under reduced pressure. The residue was purified by prep-HPLC (column: Waters xbridge 150*25 mm 10 μm; mobile phase: [water (NH₄HCO₃)-ACN]; gradient: 15%-45% B over 10 min) to give N-(4-(2-(2-aminopyridin-3-yl)-3H-imidazo[4,5-b]pyridin-3-yl)benzyl)-2-cyanothiazole-4-carboxamide (Example 244, 15 mg, yield: 17%) was obtained as a light-yellow solid. MS: m/z=453.0 [M+H]⁺. ¹H NMR (400 MHz, Dimethylsulfoxide-d₆) δ 8.49 (d, J=0.8 Hz, 1H), 8.30 (dt, J=4.8, 1.2 Hz, 1H), 8.19 (br s, 1H), 8.10 (d, J=8.0 Hz, 1H), 8.00 (d, J=4.8 Hz, 1H), 7.50 (d, J=8.0 Hz, 2H), 7.38-7.32 (m, 3H), 7.20 (d, J=7.6 Hz, 1H), 6.52 (br s, 2H), 6.38 (dd, J=7.6, 4.8 Hz, 1H), 4.64 (d, J=6.4 Hz, 2H).

### Example 245: N-(4-(2-(2-Aminopyridin-3-yl)-5-phenyl-3H-imidazo[4,5-b]pyridin-3-yl)benzyl)-2-cyanothiazole-5-carboxamide

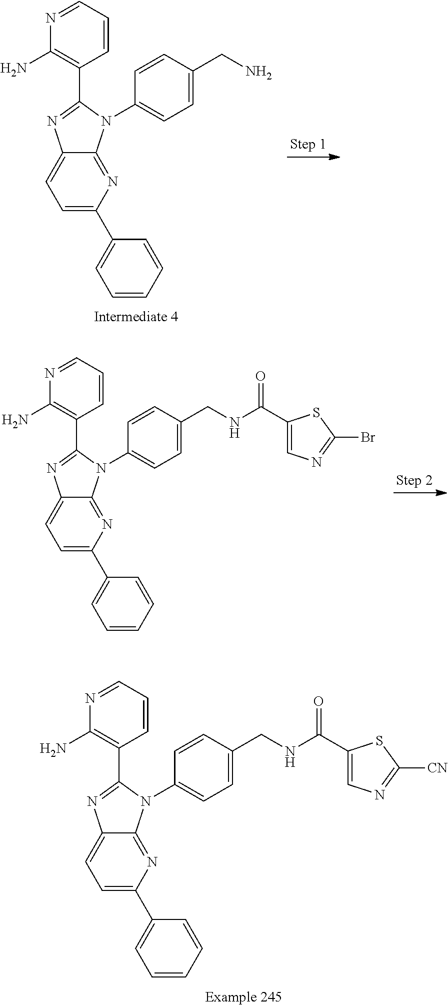 [SMILES: NCc1ccc(-n2c(-c3cccnc3N)nc3ccc(-c4ccccc4)nc32)cc1 ; Nc1ncccc1-c1nc2ccc(-c3ccccc3)nc2n1-c1ccc(CNC(=O)c2cnc(Br)s2)cc1 ; N#Cc1ncc(C(=O)NCc2ccc(-n3c(-c4cccnc4N)nc4ccc(-c5ccccc5)nc43)cc2)s1]

### Step 1: N-(4-(2-(2-Aminopyridin-3-yl)-5-phenyl-3H-imidazo[4,5-b]pyridin-3-yl)benzyl)-2-bromothiazole-5-carboxamide

To a solution of Intermediate 4 (300 mg, 764 μmol) in dimethylformamide (2 mL) were added HATU (436 mg, 1.15 mmol), DIEA (395 mg, 3 mmol) and 2-bromothiazole-5-carboxylic acid (175 mg, 841 μmol). The mixture was stirred at 25° C. for 16 hr. The reaction mixture was quenched with H₂O (10 mL) at 25° C., and then extracted with EtOAc (10 mL×4). The combined organic layers were washed with brine (10 mL×2), dried over Na₂SO₄, filtered, concentrated and purified by prep-HPLC (neutral condition: column: Waters xbridge 150*25 mm 10 μm; mobile phase: [water (NH₄HCO₃)-ACN]; B %: 40%-70%, 9 min) to give N-(4-(2-(2-aminopyridin-3-yl)-5-phenyl-3H-imidazo[4,5-b]pyridin-3-yl)benzyl)-2-bromothiazole-5-carboxamide (142 mg, yield: 32%) as a light-yellow lyophilized powder. MS: m/z=582.2, 584.2 [M+H]⁺. ¹H NMR (400 MHz, Dimethylsulfoxide-d₆) δ 9.44 (t, J=6.0 Hz, 1H), 8.33-8.24 (m, 2H), 8.05-7.97 (m, 4H), 7.50-7.44 (m, 6H), 7.41-7.37 (m, 1H), 7.22 (dd, J=7.6, 1.6 Hz, 1H), 6.93 (br s, 2H), 6.42 (dd, J=7.6, 5.2 Hz, 1H), 4.58 (d, J=6.0 Hz, 2H).

### Step 2: N-(4-(2-(2-Aminopyridin-3-yl)-5-phenyl-3H-imidazo[4,5-b]pyridin-3-yl)benzyl)-2-cyanothiazole-5-carboxamide

To a solution of N-[[4-[2-(2-amino-3-pyridyl)-5-phenyl-imidazo[4,5-b]pyridin-3-yl]phenyl]methyl]-2-bromo-thiazole-5-carboxamide (100 mg, 172 μmol) in pyridine (4 mL) was added CuCN (32.3 mg, 361 μmol) at 25° C. The mixture was stirred at 145° C. for 6 hr. Filtered and concentrated under reduced pressure. The residue was purified by prep-HPLC(column: Waters xbridge 150*25 mm 10 μm; mobile phase: [water (NH₄HCO₃)-ACN]; gradient: 35%-65% B over 10 min) to give N-(4-(2-(2-aminopyridin-3-yl)-5-phenyl-3H-imidazo[4,5-b]pyridin-3-yl)benzyl)-2-cyanothiazole-5-carboxamide (Example 245, 5 mg, yield: 5.5%) as a light-yellow solid. MS: m/z=529.2 [M+H]⁺. ¹H NMR (400 MHz, Dimethylsulfoxide-d₆) δ 8.42 (s, 1H), 8.17 (d, J=8.4 Hz, 1H), 8.05-7.88 (m, 5H), 7.54 (d, J=8.0 Hz, 2H), 7.47-7.36 (m, 5H), 7.18 (d, J=7.6 Hz, 1H), 6.52 (hr s, 2H), 6.39 (dd, J=7.6, 4.8 Hz, 1H), 4.65 (d, J=6.0 Hz, 2H).

### Example 246: N-(4-(2-(2-Aminopyridin-3-yl)-5-phenyl-3H-imidazo[4,5-b]pyridin-3-yl)benzyl)-2-cyanothiazole-4-carboxamide

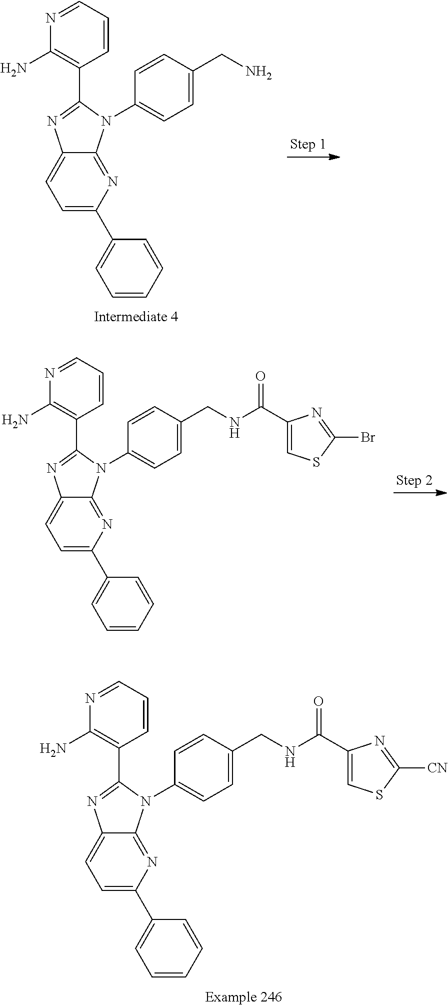 [SMILES: NCc1ccc(-n2c(-c3cccnc3N)nc3ccc(-c4ccccc4)nc32)cc1 ; Nc1ncccc1-c1nc2ccc(-c3ccccc3)nc2n1-c1ccc(CNC(=O)c2csc(Br)n2)cc1 ; N#Cc1nc(C(=O)NCc2ccc(-n3c(-c4cccnc4N)nc4ccc(-c5ccccc5)nc43)cc2)cs1]

### Step 1: N-(4-(2-(2-Aminopyridin-3-yl)-5-phenyl-3H-imidazo[4,5-b]pyridin-3-yl)benzyl)-2-bromothiazole-4-carboxamide

To a solution of Intermediate 4 (292 mg, 1.40 mmol) in DMF (1 mL) was added EDCI (366 mg, 1.91 mmol), HOBt (258 mg, 1.91 mmol), DIEA (659 mg, 5.10 mmol) and 3-[3-[4-(aminomethyl)phenyl]-5-phenyl-imidazo[4,5-b]pyridin-2-yl]pyridin-2-amine (500 mg, 1.27 mmol). The mixture was stirred at 25° C. for 12 hr. Then it was filtered and concentrated under reduced pressure. The residue was purified by silica gel flash chromatography (eluent of 0-10% MeOH in CH₂Cl₂) to give N-(4-(2-(2-aminopyridin-3-yl)-5-phenyl-3H-imidazo[4,5-b]pyridin-3-yl)benzyl)-2-bromothiazole-4-carboxamide (440 mg, 59% yield) as a yellow solid. MS: m/z=582.1, 584.1 [M+H]⁺. ¹H NMR (400 MHz, Dimethylsulfoxide-d_{f}) S 9.21 (t, J=6.4 Hz, 1H), 8.34 (s, 1H), 8.25 (d, J=8.0 Hz, 1H), 8.05-7.95 (m, 4H), 7.49-7.43 (m, 6H), 7.41-7.36 (m, 1H), 7.20 (dd, J=7.6, 2.0 Hz, 1H), 6.95 (s, 2H), 6.41 (dd, J=7.6, 4.8 Hz, 1H), 4.54 (d, J=6.4 Hz, 2H).

### Step 2: N-(4-(2-(2-Aminopyridin-3-yl)-5-phenyl-3H-imidazo[4,5-b]pyridin-3-yl)benzyl)-2-cyanothiazole-4-carboxamide

To a solution of N-[[4-[2-(2-amino-3-pyridyl)-5-phenyl-imidazo[4,5-b]pyridin-3-yl]phenyl]methyl]-2-bromo-thiazole-4-carboxamide (100 mg, 172 μmol) in pyridine (4 mL) was added CuCN (32.3 mg, 361 μmol) at 25° C. The mixture was stirred at 145° C. for 6 hr, and then was filtered and concentrated under reduced pressure. The residue was purified by prep-HPLC (column: Waters xbridge 150*25 mm 10 μm; mobile phase: [water (NH₄HCO₃)-ACN]; B %: 37%-67%, 10 min) to give N-(4-(2-(2-Aminopyridin-3-yl)-5-phenyl-3H-imidazo[4,5-b]pyridin-3-yl)benzyl)-2-cyanothiazole-4-carboxamide (Example 246, 30 mg, yield; 31%) as a light-yellow solid. MS: m/z=529.2 [M+H]⁺. ¹H NMR (400 MHz, Dimethylsulfoxide-d₆) δ 8.28 (s, 1H), 8.03-7.93 (m, 2H), 7.82-7.76 (m, 3H), 7.66 (d, J=8.4 Hz, 1H), 7.33-7.16 (m, 7H), 6.97 (dd, J=7.6, 1.6 Hz, 1H), 6.29 (br s, 2H), 6.18 (dd, J=7.6, 4.8 Hz, 1H), 4.45 (d, J=6.4 Hz, 1H).

### Example 247: 4-(4-(4-(2-(2-Aminopyridin-3-yl)-5-phenyl-3H-imidazo[4,5-b]pyridin-3-yl)benzyl)piperazine-1-carbonyl)thiazole-2-carbonitrile

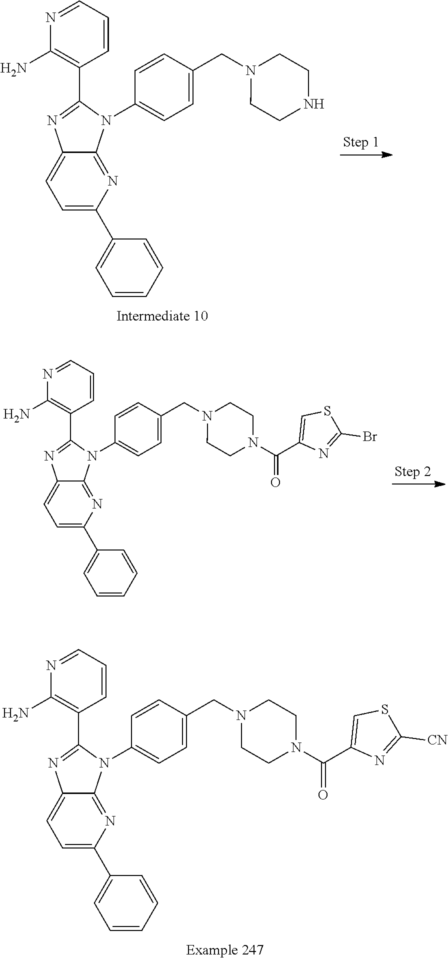 [SMILES: Nc1ncccc1-c1nc2ccc(-c3ccccc3)nc2n1-c1ccc(CN2CCNCC2)cc1 ; Nc1ncccc1-c1nc2ccc(-c3ccccc3)nc2n1-c1ccc(CN2CCN(C(=O)c3csc(Br)n3)CC2)cc1 ; N#Cc1nc(C(=O)N2CCN(Cc3ccc(-n4c(-c5cccnc5N)nc5ccc(-c6ccccc6)nc54)cc3)CC2)cs1]

### Step 1: (4-(4-(2-(2-Aminopyridin-3-yl)-5-phenyl-3H-imidazo[4,5-b]pyridin-3-yl)benzyl)piperazin-1-yl)(2-bromothiazol-4-yl)methanone

To a solution of Intermediate 10 (300 mg, 645 μmol) and 2-bromothiazole-4-carboxylic acid (149 mg, 715 μmol) in DMF (5 mL) were added EDCI (187 mg, 975 μmol), HOBt (132 mg, 975 μmol), and DIEA (336 mg, 2.60 mmol) at 0° C. The mixture was stirred at 25° C. for 12 hr, and then was filtered and concentrated under reduced pressure. The residue was purified by silica gel flash chromatography (eluent of 0-10% MeOH in CH₂Cl₂) to give (4-(4-(2-(2-Aminopyridin-3-yl)-5-phenyl-3H-imidazo[4,5-b]pyridin-3-yl)benzyl)piperazin-1-yl)2-bromothiazol-4-yl)methanone (212 mg, yield: 50%) was obtained as a yellow solid. MS: m/z=651.2, 653.1 [M+H]⁺. ¹H NMR (400 MHz, Dimethylsulfoxide-d₆) δ 8.26 (d, J=8.4 Hz, 1H), 8.13 (s, 1H), 8.05-7.95 (m, 4H), 7.51-7.37 (m, 7H), 7.15 (dd, J=7.6, 1.6 Hz, 1H), 7.02 (s, 2H), 6.39 (dd, J=7.6, 4.8 Hz, 1H), 3.66-3.60 (m, 6H), 3.34-3.29 (m, 2H), 2.47-2.43 (m, 2H).

### Step 2: 4-(4-(4-(2-(2-Aminopyridin-3-yl)-5-phenyl-3H-imidazo[4,5-b]pyridin-3-yl)benzyl)piperazine-1-carbonyl)thiazole-2-carbonitrile

To a solution of [4-[[4-[2-(2-amino-3-pyridyl)-5-phenyl-imidazo[4,5-b]pyridin-3-yl]phenyl]methyl]piperazin-1-yl]-(2-bromothiazol-4-yl)methanone (150 mg, 230 μmol) in pyridine (4 mL) was added CuCN (41.2 mg, 460 μmol) at 25° C. The mixture was stirred at 145° C. for 6 hr. The reaction mixture was filtered and concentrated under reduced pressure. The residue was purified by prep-HPLC (column: Waters xbridge 150*25 mm 10 μm; mobile phase: [water (NH₄HCO₃)-ACN]; B %: 42%-72%, 10 min) to give 4-(4-(4-(2-(2-Aminopyridin-3-yl)-5-phenyl-3H-imidazo[4,5-b]pyridin-3-yl)benzyl)piperazine-1-carbonyl)thiazole-2-carbonitrile (Example 247, 30 mg, yield: 22%) was obtained as a light-yellow solid. MS: m/z=598.2 [M+H]⁺. ¹H NMR (400 MHz, Dimethylsulfoxide-d₆) δ 8.02 (s, 1H), 7.97 (d, J=8.4 Hz, 1H), 7.83-7.80 (m, 3H), 7.69 (d, J=8.8 Hz, 1H), 7.34-7.20 (m, 7H), 6.94 (dd, J=7.6, 1.6 Hz, 1H), 6.38 (br s, 2H), 6.17 (dd, J=7.6, 4.8 Hz, 1H), 3.53 (s, 2H), 3.47-3.43 (m, 4H), 2.37-2.33 (m, 2H), 2.30-2.25 (m, 2H).

### Example 248: 4-((7-(4-(2-(2-aminopyridin-3-yl)-5-phenyl-3H-imidazo[4,5-b]pyridin-3-yl)benzyl)-7-azaspiro[3.5]nonan-2-yl)amino)-1,3,5-triazine-2-carbonitrile

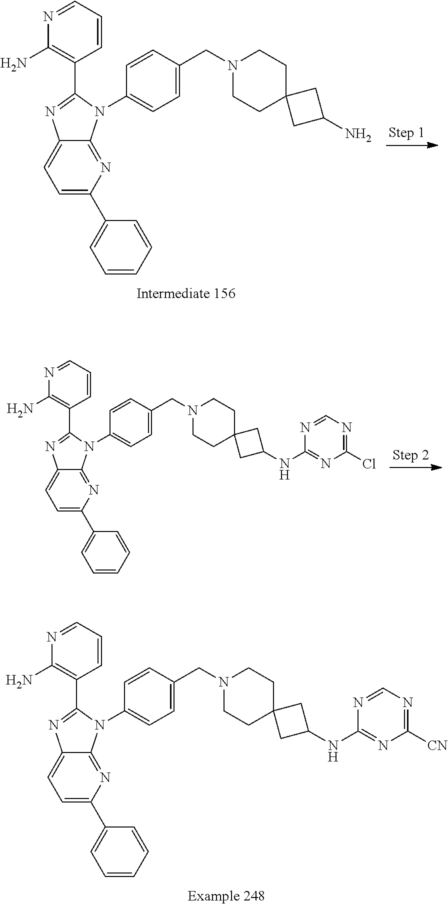 [SMILES: Nc1ncccc1-c1nc2ccc(-c3ccccc3)nc2n1-c1ccc(CN2CCC3(CC2)CC(N)C3)cc1 ; Nc1ncccc1-c1nc2ccc(-c3ccccc3)nc2n1-c1ccc(CN2CCC3(CC2)CC(Nc2ncnc(Cl)n2)C3)cc1 ; N#Cc1ncnc(NC2CC3(CCN(Cc4ccc(-n5c(-c6cccnc6N)nc6ccc(-c7ccccc7)nc65)cc4)CC3)C2)n1]

### Step 1: 7-(4-(2-(2-Ainopyridin-3-yl)-5-phenyl-3H-imidazo[4,5-b]pyridin-3-yl)benzyl)-N-(4-chloro-1,3,5-triazin-2-yl)-7-azaspiro[3.5]nonan-2-amine

To a solution of Intermediate 156 (100 mg, 181 μmol) in THF (20 mL) was added DIEA (70.2 mg, 543 μmol). The mixture was stirred at 0° C. for 10 min. Then 2,4-dichloro-1,3,5-triazine (32.6 mg, 217 μmol) in THF (1 mL) was added to the mixture. The mixture was stirred at 0° C. for 0.5 hr, then was partitioned between EtOAc (50 mL) and H₂O (30 mL). The organic layer was washed with brine (60 mL) and dried over Na₂SO₄, filtered, and concentrated. The residue was purified by prep-TLC (CH₂OC₂:MeOH=10:1) to give 7-(4-(2-(2-ainopyridin-3-yl)-5-phenyl-3H-imidazo[4,5-b]pyridin-3-yl)benzyl)-N-(4-chloro-1,3,5-triazin-2-yl)-7-azaspiro[3.5]nonan-2-amine (8.4 mg, yield: 7.1%) as a light-yellow solid. MS: m/z=629.3 [M+H]⁺. ¹H NMR (400 MHz, Dimethylsulfoxide-d₆) δ 8.95-8.93 (m, 1H), 8.51-8.31 (m, 1H), 8.27 (d, J=8.4 Hz, 1H), 8.04-7.96 (m, 4H), 7.48-7.37 (m, 7H), 7.16-7.14 (m, 1H), 7.02 (br s, 2H), 6.37 (dd, J=7.6, 4.8 Hz, 1H), 4.13-4.03 (m, 1H), 3.53 (s, 2H), 2.34-2.23 (m, 4H), 2.21-2.15 (m, 2H), 1.80-1.73 (m, 2H), 1.63-1.52 (m, 4H).

### Step 2: 4-((7-(4-(2-(2-aminopyridin-3-yl)-5-phenyl-3H-imidazo[4,5-b]pyridin-3-yl)benzyl)-7-azaspiro[3.5]nonan-2-yl)amino)-1,3,5-triazine-2-carbonitrile

To a solution of 7-(4-(2-(2-aminopyridin-3-yl)-5-phenyl-3H-imidazo[4,5-b]pyridin-3-yl)benzyl)-N-(4-chloro-1,3,5-triazin-2-yl)-7-azaspiro[3.5]nonan-2-amine (50 mg, 79.5 μmol) in DMSO (2 mL) were added KCN (30 mg, 461 μmol) and DABCO (1.78 mg, 15.9 μmol). The mixture was stirred at 25° C. for 1 hr. The mixture was quenched with H₂O (10 mL) and extracted with EtOAc (30 mL), the combined organic phases were dried over anhydrous sodium sulfate, filtered and concentrated. The residue was purified by prep-TLC (CH₂Cl₂:MeOH=10:1) to give 4-((7-(4-(2-(2-aminopyridin-3-yl)-5-phenyl-3H-imidazo[4,5-b]pyridin-3-yl)benzyl)-7-azaspiro[3.5]nonan-2-yl)amino)-1,3,5-triazine-2-carbonitrile (Example 248, 6.3 mg, yield: 12.3%) as a light-yellow solid. MS: m/z=620.4 [M+H]⁺. ¹H NMR (400 MHz, Chloroform-d) δ 8.72-8.46 (m, 1H), 8.14 (d, J=8.4 Hz, 1H), 8.08-8.05 (m, 1H), 8.02 (d, J=8.0 Hz, 2H), 7.81 (d, J=8.4 Hz, 1H), 7.67-7.51 (m, 2H), 7.48-7.41 (m, 4H), 7.39-7.35 (m, 1H), 7.08 (d, J=7.6 Hz, 1H), 6.60 (br s, 2H), 6.38-6.33 (m, 1H), 5.99-5.91 (m, 1H), 4.50-4.41 (m, 1H), 3.77-3.54 (m, 2H), 2.55-2.39 (m, 4H), 1.65-1.53 (m, 8H).

### Example 249: 4-(7-(4-(2-(2-aminopyridin-3-yl)-5-phenyl-3H-imidazo[4,5-b]pyridin-3-yl)benzyl)-2,7-diazaspiro[3.5]nonan-2-yl)-1,3,5-triazine-2-carbonitrile

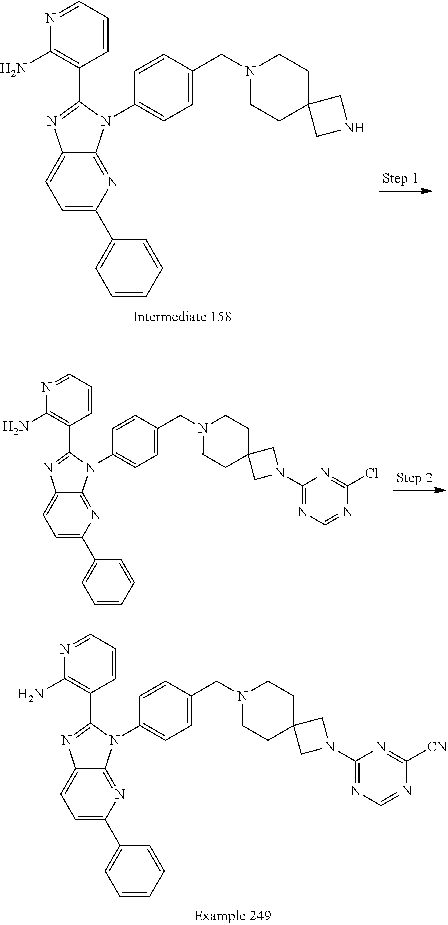

### Step 1: 3-(3-(4-((2-(4-Chloro-1,3,5-triazin-2-yl)-2,7-diazaspiro[3.5]nonan-7-yl)methyl)phenyl)-5-phenyl-3H-imidazo[4,5-b]pyridin-2-yl)pyridin-2-amine

To a solution of Intermediate 158 (100 mg, 186 μmol) in THF (20 mL) was added DIEA (72.1 mg, 558 μmol). The mixture was stirred at 0° C. for 10 min. Then 2,4-dichloro-1,3,5-triazine (33.4 mg, 223 μmol) in THF (1 mL) was added to the mixture. The mixture was stirred at 0° C. for 0.5 hr, and then was partitioned between EtOAc (50 mL) and H₂O (30 mL). The organic layer was washed with brine (60 mL) and dried over Na₂SO₄, filtered, and concentrated. The residue was purified by prep-TLC (CH₂Cl₂:MeOH=10:1) to give 3-(3-(4-((2-(4-chloro-1,3,5-triazin-2-yl)-2,7-diazaspiro[3.5]nonan-7-yl)methyl)phenyl)-5-phenyl-3H-imidazo[4,5-b]pyridin-2-yl)pyridin-2-amine (12.0 mg, yield: 10%) as a light-yellow solid. MS: m/z=615.3 [M+H]⁺. ¹H NMR (400 MHz, Dimethylsulfoxide-d₆) δ 8.43 (s, 1H), 8.27 (d, J=8.4 Hz, 1H), 8.05-7.97 (m, 4H), 7.50-7.39 (m, 7H), 7.18-7.14 (m, 1H), 7.03 (br s, 2H), 6.38 (dd, J=7.6, 4.8 Hz, 1H), 3.84 (d, J=4.8 Hz, 4H), 3.56 (s, 2H), 2.40-2.31 (m, 4H), 1.81-1.74 (m, 4H).

### Step 2: 4-(7-(4-(2-(2-Aminopyridin-3-yl)-5-phenyl-3H-imidazo[4,5-b]pyridin-3-yl)benzyl)-2,7-diazaspiro[3.5]nonan-2-yl)-1,3,5-triazine-2-carbonitrile

To a solution of 7-(4-(2-(2-aminopyridin-3-yl)-5-phenyl-3H-imidazo[4,5-b]pyridin-3-yl)benzyl)-N-(4-chloro-1,3,5-triazin-2-yl)-7-azaspiro[3.5]nonan-2-amine (40 mg, 67.9 μmol) in DMSO (2 mL) were added KCN (8.84 mg, 136 μmol) and DABCO (1.52 mg, 13.6 μmol). The mixture was stirred at 25° C. for 1 hr. The reaction mixture was quenched with H₂O (10 mL) and extracted with EtOAc (30 mL). The combined organic layers were dried over anhydrous sodium sulfate, filtered and concentrated. The residue was purified by prep-TLC (CH₂Cl₂:MeOH=10:1) to give 4-(7-(4-(2-(2-aminopyridin-3-yl)-5-phenyl-3H-imidazo[4,5-b]pyridin-3-yl)benzyl)-2,7-diazaspiro[3.5]nonan-2-yl)-1,3,5-triazine-2-carbonitrile (Example 249, 22.9 mg, yield: 25%) as a light-yellow solid. MS: m/z=606.4 [M+H]⁺. ¹H NMR (400 MHz, Dimethylsulfoxide-d₆) δ 8.68 (s, 1H), 8.27 (d, J=8.4 Hz, 1H), 8.03-7.98 (m, 4H), 7.48-7.44 (m, 6H), 7.42-7.38 (m, 1H), 7.16 (dd, J=7.6, 1.6 Hz, 1H), 7.03 (br s, 2H), 6.38 (dd, J=7.6, 4.8 Hz, 1H), 3.87 (s, 4H), 3.58 (s, 2H), 2.42-2.34 (m, 4H), 1.82-1.48 (m, 4H).

### Example 250: 4-(4-(4-(2-(2-Aminopyridin-3-yl)-5-phenyl-3H-imidazo[4,5-b]pyridin-3-yl)benzyl)-1,4-diazepan-1-yl)-1,3,5-triazine-2-carbonitrile

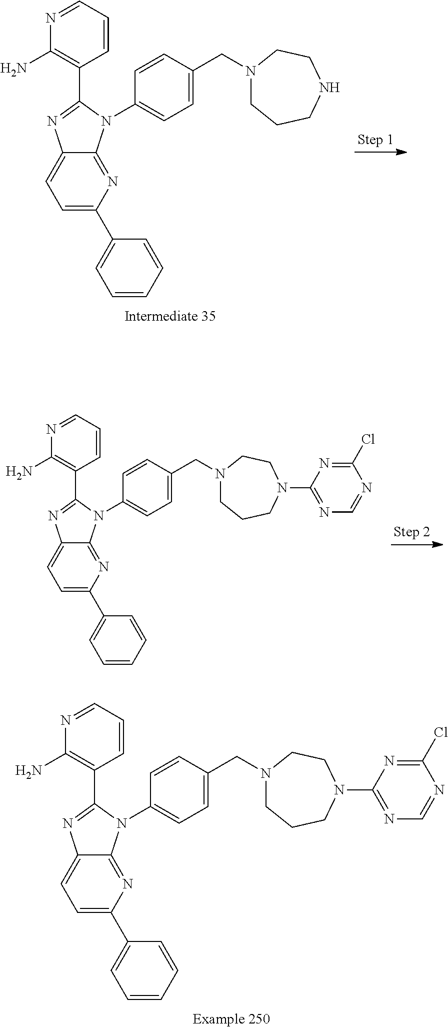

### Step 1: 3-(3-(4-((4-(4-Chloro-1,3,5-triazin-2-yl)-1,4-diazepan-1-yl)methyl)phenyl)-5-phenyl-3H-imidazo[4,5-b]pyridin-2-yl)pyridin-2-amine

To a solution of Intermediate 35 (100 mg, 195 μmol) in THF (20 mL) was added DIEA (75.7 mg, 586 μmol). The mixture was stirred at 0° C. for 10 min. Then 2,4-dichloro-1,3,5-triazine (35.2 mg, 234 μmol) in THF (1 mL) was added to the mixture. The mixture was stirred at 0° C. for 0.5 hr. The mixture was partitioned between EtOAc (50 mL) and H₂O (30 mL). The separated organic layer was washed with brine (60 mL) and dried over Na₂SO₄, filtered and concentrated. The residue was purified by prep-TLC (CH₂Cl₂:MeOH=10:1) to give 3-(3-(4-((4-(4-chloro-1,3,5-triazin-2-yl)-1,4-diazepan-1-yl)methyl)phenyl)-5-phenyl-3H-imidazo[4,5-b]pyridin-2-yl)pyridin-2-amine (9.2 mg, yield: 7.7%) was obtained as a light-yellow solid. MS: m/z=589.3 [M+H]⁺. ¹H NMR (400 MHz, Dimethylsulfoxide-d₆) δ 8.48 (d, J=8.4 Hz, 1H), 8.26 (d, J=8.4 Hz, 1H), 8.05-7.97 (m, 4H), 7.50-7.39 (m, 7H), 7.16-7.11 (m, 1H), 7.04 (br s, 2H), 6.39-6.30 (m, 1H), 3.86-3.78 (m, 4H), 3.74-3.72 (m, 2H), 2.80-2.75 (m, 2H), 2.65-2.62 (m, 2H), 1.88-1.85 (m, 2H).

### Step 2: 4-(4-(4-(2-(2-Aminopyridin-3-yl)-5-phenyl-3H-imidazo[4,5-b]pyridin-3-yl)benzyl)-1,4-diazepan-1-yl)-1,3,5-triazine-2-carbonitrile

To a solution of 3-(3-(4-((4-(4-chloro-1,3,5-triazin-2-yl)-1,4-diazepan-1-yl)methyl)phenyl)-5-phenyl-3H-imidazo[4,5-b]pyridin-2-yl)pyridin-2-amine (90 mg, 146 μmol) in DMSO (2 mL) were added KCN (19.1 mg, 293 μmol) and DABCO (3.28 mg, 29.3 μmol). The mixture was stirred at 25° C. for 1 hr. The mixture was quenched with H₂O (10 mL) and extracted with EtOAc (30 mL), the combined organic phases were dried over anhydrous Na₂SO₄, filtered and concentrated. The residue was purified by prep-TLC (CH₂Cl₂:MeOH=10:1) to give 4-(4-(4-(2-(2-aminopyridin-3-yl)-5-phenyl-3H-imidazo[4,5-b]pyridin-3-yl)benzyl)-1,4-diazepan-1-yl)-1,3,5-triazine-2-carbonitrile (Example 250, 6.5 mg, yield: 15.7%) as a light-yellow solid. MS: m/z=580.3 [M+H]⁺. ¹H NMR (400 MHz, Chloroform-d) δ 8.56 (d, J=10.4 Hz, 1H), 8.14 (d, J=8.4 Hz, 1H), 8.07-8.05 (m, 1H), 8.03-8.00 (m, 3H), 7.81 (d, J=8.4 Hz, 1H), 7.51-7.48 (m, 2H), 7.44-7.40 (m, 4H), 7.13-7.09 (m, 1H), 6.75 (br s, 2H), 6.39-6.35 (m, 1H), 3.95-3.91 (m, 2H), 3.90-3.87 (m, 2H), 3.77 (s, 2H), 2.87-2.83 (m, 2H), 2.74-2.70 (m, 2H), 2.02-2.00 (m, 2H).

### Example 251: 4-(7-(4-(2-(2-aminopyridin-3-yl)-5-phenyl-3H-imidazo[4,5-b]pyridin-3-yl)benzyl)-2,7-diazaspiro[4.4]nonan-2-yl)-1,3,5-triazine-2-carbonitrile

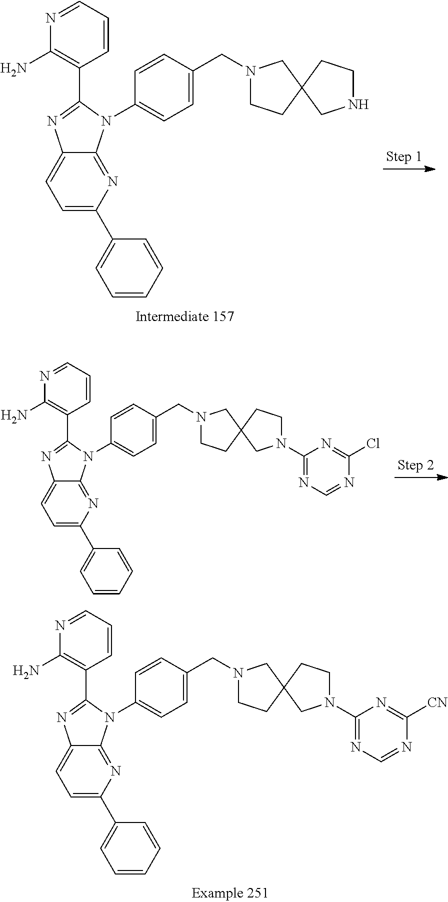

### Step 1: 3-(3-(4-((7-(4-Chloro-1,3,5-triazin-2-yl)-2,7-diazaspiro[4.4]nonan-2-yl)methyl)phenyl)-5-phenyl-3H-imidazo[4,5-b]pyridin-2-yl)pyridin-2-amine

To a solution of Intermediate 157 (120 mg, 239 μmol) in THF (10 mL) was added DIEA (93 mg, 718 μmol). The 2,4-dichloro-1,3,5-triazine (39 mg, 263 μmol) in THF (2 mL) was added into the mixture at 0° C. The mixture was stirred at 0° C. for 0.5 hr. The mixture was concentrated under reduced pressure at 40° C. After purified by prep-TLC (CH₂Cl₂:MeOH=20:1), 3-(3-(4-((7-(4-chloro-1,3,5-triazin-2-yl)-2,7-diazaspiro[4.4]nonan-2-yl)methyl)phenyl)-5-phenyl-3H-imidazo[4,5-b]pyridin-2-yl)pyridin-2-amine (80 mg, yield: 49%) was obtained as a yellow oil. MS: m/z=615.2 [M+H]⁺.

### Step 2: 4-(7-(4-(2-(2-Aminopyridin-3-yl)-5-phenyl-3H-imidazo[4,5-b]pyridin-3-yl)benzyl)-2,7-diazaspiro[4.4]nonan-2-yl)-1,3,5-triazine-2-carbonitrile

To a solution of 3-(3-(4-((7-(4-chloro-1,3,5-triazin-2-yl)-2,7-diazaspiro[4.4]nonan-2-yl)methyl)phenyl)-5-phenyl-3H-imidazo[4,5-b]pyridin-2-yl)pyridin-2-amine (80 mg, 130 μmol) and KCN (17 mg, 261 μmol) in DMSO (2 mL) was added DABCO (2.9 mg, 26 μmol). The mixture was stirred at 25° C. for 2 hr. The reaction mixture was poured into water (5 mL). The resulting mixture was extracted with EtOAc (6 mL). The organic layer was washed with brine (5 mL), dried over anhydrous Na₂SO₄, filtered and concentrated to dryness. The residue was purified by silica gel flash chromatography (Eluent of 0˜7% MeOH in CH₂Cl₂) to give 4-(7-(4-(2-(2-aminopyridin-3-yl)-5-phenyl-3H-imidazo[4,5-b]pyridin-3-yl)benzyl)-2,7-diazaspiro[4.4]nonan-2-yl)-1,3,5-triazine-2-carbonitrile (Example 251, 23.0 mg, yield: 28%) as a light-yellow solid. MS: m/z=606.4 [M+H]⁺. ¹H NMR (400 MHz, Acetonitrile-d₃) S 8.51 (d, J=8.4 Hz, 1H), 8.14 (d, J=8.4 Hz, 1H), 8.04-7.95 (m, 3H), 7.87 (d, J=8.4 Hz, 1H), 7.51-7.37 (m, 7H), 7.13-7.11 (m, 1H), 6.60 (br s, 2H), 6.31 (dd, J=7.6, 4.8 Hz, 1H), 3.73-3.67 (m, 2H), 3.64-3.55 (m, 2H), 3.54-3.42 (m, 2H), 2.76-2.69 (m, 1H), 2.67-2.61 (m, 1H), 2.59-2.55 (m, 1H), 2.51-2.47 (m, 1H), 2.08-1.97 (m, 2H), 1.89-1.82 (m, 2H).

### Example 252: 4-(2-(4-(2-(2-Aminopyridin-3-yl)-5-phenyl-3H-imidazo[4,5-b]pyridin-3-yl)benzyl)-2,8-diazaspiro[4.5]decan-8-yl)-1,3,5-triazine-2-carbonitrile

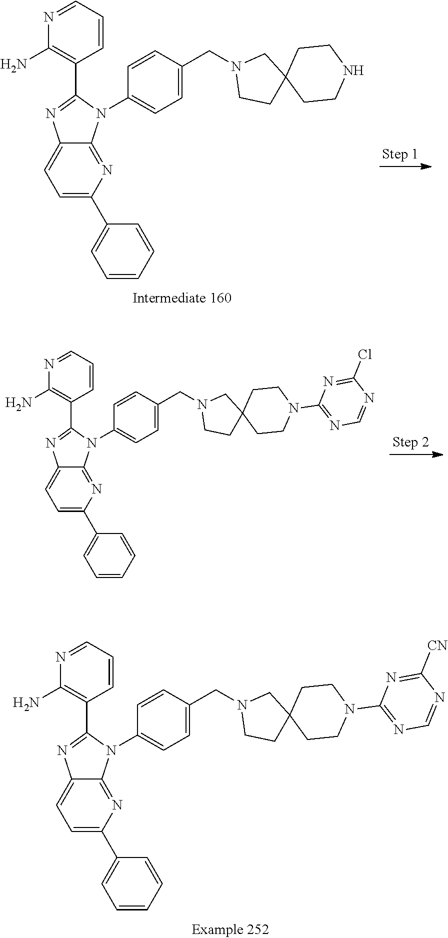 [SMILES: Nc1ncccc1-c1nc2ccc(-c3ccccc3)nc2n1-c1ccc(CN2CCC3(CCNCC3)C2)cc1 ; Nc1ncccc1-c1nc2ccc(-c3ccccc3)nc2n1-c1ccc(CN2CCC3(CCN(c4ncnc(Cl)n4)CC3)C2)cc1 ; N#Cc1ncnc(N2CCC3(CCN(Cc4ccc(-n5c(-c6cccnc6N)nc6ccc(-c7ccccc7)nc65)cc4)C3)CC2)n1]

### Step 1: 3-(3-(4-((8-(4-Chloro-1,3,5-triazin-2-yl)-2,8-diazaspiro[4.5]decan-2-yl)methyl)phenyl)-5-phenyl-3H-imidazo[4,5-b]pyridin-2-yl)pyridin-2-amine

To a solution of Intermediate 160 (150 mg, 291 μmol) in THF (10 mL) was added DIEA (113 mg, 873 μmol). The 2,4-dichloro-1,3,5-triazine (48 mg, 320 μmol) in THF (2 mL) was added into the mixture at 0° C. The mixture was stirred at 0° C. for 0.5 hr. The mixture was concentrated under reduced pressure at 40° C. The residue was purified by silica gel flash chromatography (Eluent of 0˜7% MeOH in CH₂Cl₂) to give 3-(3-(4-((8-(4-chloro-1,3,5-triazin-2-yl)-2,8-diazaspiro[4.5]decan-2-yl)methyl)phenyl)-5-phenyl-3H-imidazo[4,5-b]pyridin-2-yl)pyridin-2-amine (140 mg, yield: 65%) as a yellow oil. MS: m/z=629.2 [M+H]⁺.

### Step 2: 4-(2-(4-(2-(2-Aminopyridin-3-yl)-5-phenyl-3H-imidazo[4,5-b]pyridin-3-yl)benzyl)-2,8-diazaspiro[4.5]decan-8-yl)-1,3,5-triazine-2-carbonitrile

To a solution of 3-(3-(4-((8-(4-chloro-1,3,5-triazin-2-yl)-2,8-diazaspiro[4.5]decan-2-yl)methyl)phenyl)-5-phenyl-3H-imidazo[4,5-b]pyridin-2-yl)pyridin-2-amine (80 mg, 130 μmol) and KCN (40 mg, 614 μmol) in DMSO (2 mL) was added DABCO (5 mg, 45 μmol). The mixture was stirred at 25° C. for 2 hr. The reaction mixture was poured into water (5 mL). The resulting mixture was extracted with EtOAc (6 mL). The organic layer was washed with brine (2 ml) and dried over anhydrous Na₂SO₄, filtered and concentrated to dryness. The residue was purified by prep-HPLC (column: Phenomenex C18 150*25 mm*10 μm; mobile phase: [water (NH₄HCO₃)-ACN]; gradient: 52%-82% B over 10 min) to give 4-(2-(4-(2-(2-aminopyridin-3-yl)-5-phenyl-3H-imidazo[4,5-b]pyridin-3-yl)benzyl)-2,8-diazaspiro[4.5]decan-8-yl)-1,3,5-triazine-2-carbonitrile (Example 252, 2.6 mg, yield: 1.8%) as an off-white solid. MS: m/z=620.3 [M+H]⁺. ¹H NMR (400 MHz, Acetonitrile-d₃) δ 8.52 (s, 1H), 8.17 (d, J=8.4 Hz, 1H), 8.07-7.94 (m, 3H), 7.90 (d, J=8.4 Hz, 1H), 7.53 (d, J=8.4 Hz, 2H), 7.47-7.37 (m, 5H), 7.17 (dd, J=7.6, 1.6 Hz, 1H), 6.57 (br s, 2H), 6.37 (dd, J=7.6, 4.8 Hz, 1H), 3.92-3.75 (m, 4H), 3.73 (s, 2H), 2.70 (t, J=6.4 Hz, 2H), 2.52 (s, 2H), 1.72-1.69 (m, 2H), 1.67-1.62 (m, 4H).

### Example 253: 4-(2-(4-(2-(2-Aminopyridin-3-yl)-5-phenyl-3H-imidazo[4,5-b]pyridin-3-yl)benzyl)-2,7-diazaspiro[4.5]decan-7-yl)-1,3,5-triazine-2-carbonitrile

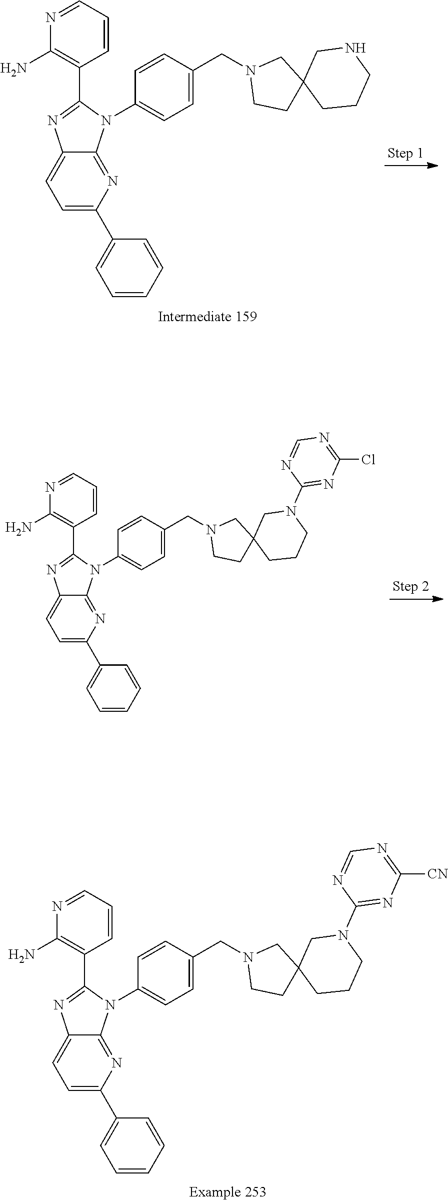 [SMILES: Nc1ncccc1-c1nc2ccc(-c3ccccc3)nc2n1-c1ccc(CN2CCC3(CCCNC3)C2)cc1 ; Nc1ncccc1-c1nc2ccc(-c3ccccc3)nc2n1-c1ccc(CN2CCC3(CCCN(c4ncnc(Cl)n4)C3)C2)cc1 ; N#Cc1ncnc(N2CCCC3(CCN(Cc4ccc(-n5c(-c6cccnc6N)nc6ccc(-c7ccccc7)nc65)cc4)C3)C2)n1]

### Step 1: 3-(3-(4-(7-(4-Chloro-1,3,5-triazin-2-yl)-2,7-diazaspiro[4.5]decan-2-yl)phenyl)-5-phenyl-3H-imidazo[4,5-b]pyridin-2-yl)pyridin-2-amine

To a solution of Intermediate 159 (150 mg, 291 μmol) in THF (8 mL) was added DIEA (113 mg, 873 μmol). The 2,4-dichloro-1,3,5-triazine (48 mg, 320 μmol) in THF (2 mL) was added into the mixture at 0° C. The mixture was stirred at 0° C. for 1 hr. The mixture was concentrated under reduced pressure at 42° C. The residue was purified by silica gel flash chromatography (Eluent of 0˜6% MeOH in CH₂Cl₂) to give 3-(3-(4-(7-(4-chloro-1,3,5-triazin-2-yl)-2,7-diazaspiro[4.5]decan-2-yl)phenyl)-5-phenyl-3H-imidazo[4,5-b]pyridin-2-yl)pyridin-2-amine (100 mg, yield: 49%) as a yellow oil. MS: m/z=629.2 [M+H]⁺.

### Step 2: 4-(2-(4-(2-(2-aminopyridin-3-yl)-5-phenyl-3H-imidazo[4,5-b]pyridin-3-yl)benzyl)-2,7-diazaspiro[4.5]decan-7-yl)-1,3,5-triazine-2-carbonitrile

To a solution of 3-(3-(4-(7-(4-chloro-1,3,5-triazin-2-yl)-2,7-diazaspiro[4.5]decan-2-yl)phenyl)-5-phenyl-3H-imidazo[4,5-b]pyridin-2-yl)pyridin-2-amine (100 mg, 160 μmol) and KCN (50 mg, 770 μmol) in DMSO (2 mL) was added DABCO (3.6 mg, 32 μmol). The mixture was stirred at 25° C. for 0.5 hr. The reaction mixture was poured into water (5 mL). The resulting mixture was extracted with EtOAc (6 mL). The organic layer was washed with brine (2 ml) and dried over anhydrous Na₂SO₄, filtered and concentrated to dryness. The residue was purified by silica gel flash chromatography (Eluent of 0˜8% MeOH in CH₂Cl₂) to give 4-(2-(4-(2-(2-aminopyridin-3-yl)-5-phenyl-3H-imidazo[4,5-b]pyridin-3-yl)benzyl)-2,7-diazaspiro[4.5]decan-7-yl)-1,3,5-triazine-2-carbonitrile (Example 253, 18.5 mg, yield: 18%) as a light-yellow solid. MS: m/z=620.5 [M+H]⁺. ¹H NMR (400 MHz, Acetonitrile-d₃) S 8.60-8.37 (m, 1H), 8.17 (dd, J=8.4, 1.2 Hz, 1H), 8.06-7.96 (m, 3H), 7.89 (d, J=8.4 Hz, 1H), 7.53-7.37 (m, 7H), 7.21-7.08 (m, 1H), 6.61 (br s, 2H), 6.33 (dd, J=7.6, 4.8 Hz, 1H), 3.95-3.82 (m, 2H), 3.80-3.73 (m, 1H), 3.69-3.62 (m, 2H), 3.61-3.51 (m, 1H), 2.89-2.83 (m, 1H), 2.58 (d, J=9.2 Hz, 1H), 2.52-2.48 (m, 1H), 1.97-1.95 (m, 1H), 1.73-1.63 (m, 4H), 1.61-1.53 (m, 2H).

### Example 254: 4-(9-(4-(2-(2-Aminopyridin-3-yl)-5-phenyl-3H-imidazo[4,5-b]pyridin-3-yl)benzyl)-2,9-diazaspiro[5.5]undecan-2-yl)-1,3,5-triazine-2-carbonitrile

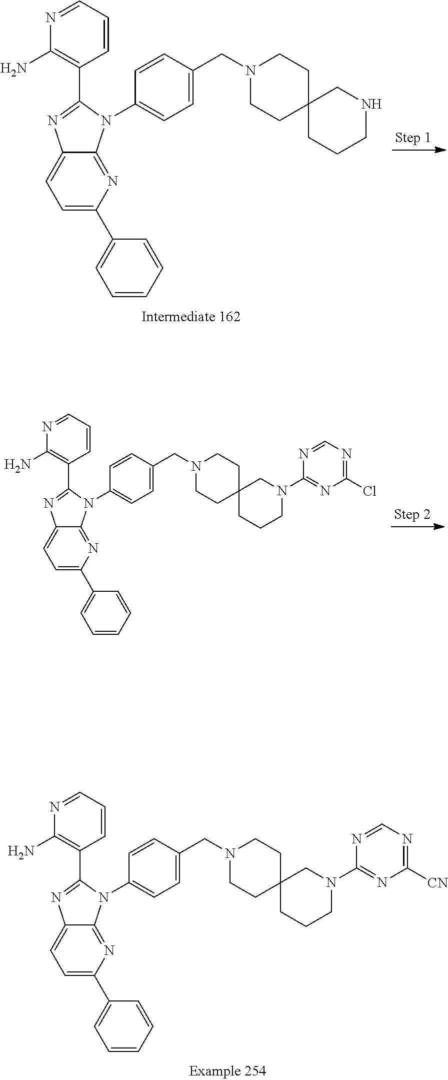 [SMILES: Nc1ncccc1-c1nc2ccc(-c3ccccc3)nc2n1-c1ccc(CN2CCC3(CCCNC3)CC2)cc1 ; Nc1ncccc1-c1nc2ccc(-c3ccccc3)nc2n1-c1ccc(CN2CCC3(CCCN(c4ncnc(Cl)n4)C3)CC2)cc1 ; N#Cc1ncnc(N2CCCC3(CCN(Cc4ccc(-n5c(-c6cccnc6N)nc6ccc(-c7ccccc7)nc65)cc4)CC3)C2)n1]

### Step 1: 3-(3-(4-((2-(4-Chloro-1,3,5-triazin-2-yl)-2,9-diazaspiro[5.5]undecan-9-yl)methyl)phenyl)-5-phenyl-3H-imidazo[4,5-b]pyridin-2-yl)pyridin-2-amine

To a mixture of Intermediate 162 (100 mg, 189 μmol) in THF (2 mL) and H₂O (0.5 mL) was added K₂CO₃ (78.3 mg, 566 μmol) at 0° C. The mixture was stirred at 0° C. for 15 minutes and 2,4-dichloro-1,3,5-triazine (28.3 mg, 189 μmol) was added. The mixture was stirred at 0° C. for 15 minutes, and then was quenched with H₂O (20 mL), extracted with EtOAc (20 mL×3). The combined organic phases were washed with brine (20 mL), dried over anhydrous Na₂SO₄, filtered and concentrated. The mixture was purified by silica gel flash chromatography (eluent 0-8% MeOH in CH₂Cl₂) and prep-TLC (10% MeOH in CH₂Cl₂) to give 3-(3-(4-((2-(4-chloro-1,3,5-triazin-2-yl)-2,9-diazaspiro[5.5]undecan-9-yl)methyl)phenyl)-5-phenyl-3H-imidazo[4,5-b]pyridin-2-yl)pyridin-2-amine (17.4 mg, yield: 29%) as a yellow solid. MS: m/z=665.3 [M+Na]⁺. ¹H NMR (400 MHz, Chloroform-d) δ 8.25 (d, J=2.0 Hz, 1H), 8.06 (d, J=8.0 Hz, 1H), 8.00-7.93 (m, 3H), 7.73 (d, J=8.4 Hz, 1H), 7.50-7.44 (m, 2H), 7.38-7.29 (m, 5H), 7.06-6.98 (m, 1H), 6.54 (br s, 2H), 6.33-6.25 (m, 1H), 3.77-3.59 (m, 6H), 2.63-2.38 (m, 4H), 1.61-1.48 (m, 8H).

### Step 2: 4-(9-(4-(2-(2-Aminopyridin-3-yl)-5-phenyl-3H-imidazo[4,5-b]pyridin-3-yl)benzyl)-2,9-diazaspiro[5.5]undecan-2-yl)-1,3,5-triazine-2-carbonitrile

To a solution of 3-[3-[4-[[2-(4-chloro-1,3,5-triazin-2-yl)-2,9-diazaspiro[5.5]undecan-9-yl]methyl]phenyl]-5-phenyl-imidazo[4,5-b]pyridin-2-yl]pyridin-2-amine (150 mg, 233 μmol) in DMSO (dry) (5 mL) were added KCN (330 mg, 5.07 mmol) and DABCO (5.23 mg, 46.6 μmol) at 20° C. The mixture was stirred at 20° C. for 12 hr. Water (10 mL) was added to the mixture. The aqueous layer was extracted with EtOAc (20 mL×2). The combined organic layers were washed with H₂O (15 mL×2), brine (15 mL), dried over anhydrous Na₂SO₄, filtered and concentrated. The residue was purified by prep-TLC (CH₂Cl₂:MeOH=10:1) to give 4-(9-(4-(2-(2-aminopyridin-3-yl)-5-phenyl-3H-imidazo[4,5-b]pyridin-3-yl)benzyl)-2,9-diazaspiro[5.5]undecan-2-yl)-1,3,5-triazine-2-carbonitrile (Example 254, 38.1 mg, yield: 26%) as a yellow solid. MS: m/z=634.3 [M+H]⁺. ¹H NMR (400 MHz, Dimethylsulfoxide-d₆) δ 8.67 (d, J=3.2 Hz, 1H), 8.27 (d, J=8.4 Hz, 1H), 8.03-7.97 (m, 4H), 7.47-7.37 (m, 7H), 7.18-7.13 (m, 1H), 7.04 (d, J=6.4 Hz, 2H), 6.36 (dd, J=7.6, 4.8 Hz, J H), 3.80-3.78 (m, 1H), 3.73 (s, 2H), 3.67-3.65 (m, 1H), 3.59 (d, J=8.8 Hz, 2H), 2.46-2.44 (m, 2H), 2.38-2.30 (m, 2H), 1.61-1.51 (m, 4H), 1.48-1.38 (m, 4H).

### Example 255: (R)-4-(7-(4-(2-(2-aminopyridin-3-yl)-5-phenyl-3H-imidazo[4,5-b]pyridin-3-yl)benzyl)-2,7-diazaspiro[4.4]nonan-2-yl)-1,3,5-triazine-2-carbonitrile

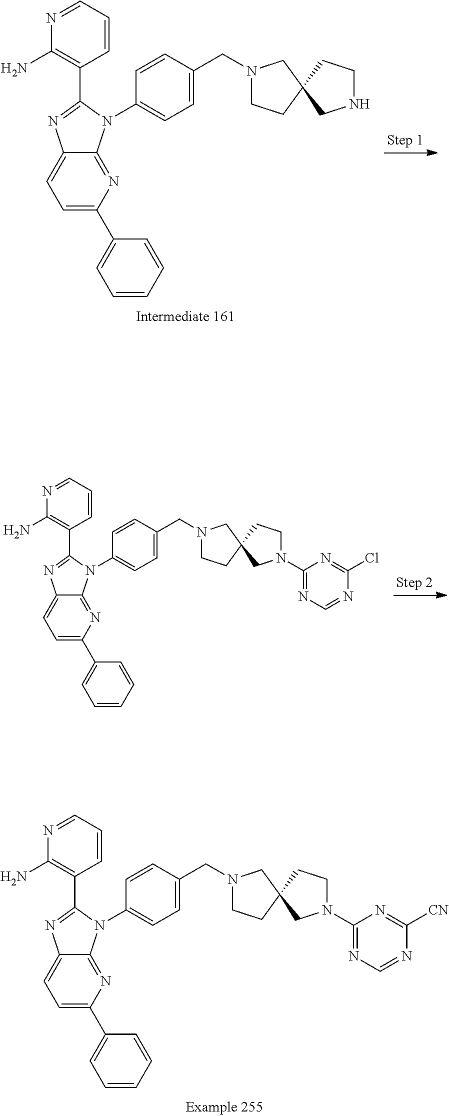 [SMILES: Nc1ncccc1-c1nc2ccc(-c3ccccc3)nc2n1-c1ccc(CN2CC[C@@]3(CCNC3)C2)cc1 ; Nc1ncccc1-c1nc2ccc(-c3ccccc3)nc2n1-c1ccc(CN2CC[C@@]3(CCN(c4ncnc(Cl)n4)C3)C2)cc1 ; N#Cc1ncnc(N2CC[C@@]3(CCN(Cc4ccc(-n5c(-c6cccnc6N)nc6ccc(-c7ccccc7)nc65)cc4)C3)C2)n1]

### Step 1: (R)-3-(3-(4-((7-(4-chloro-1,3,5-triazin-2-yl)-2,7-diazaspiro[4.4]nonan-2-yl)methyl)phenyl)-5-phenyl-3H-imidazo[4,5-b]pyridin-2-yl)pyridin-2-amine

To a solution of Intermediate 161 (200 mg, 372 μmol, HCl salt) in THF (8 mL) and H₂O (2 mL) were added K₂CO₃ (154 mg, 1.12 mmol) and 2,4-dichloro-1,3,5-triazine (61.3 mg, 409 μmol) at 0° C. This mixture was stirred at 0° C. for 0.5 hr and then H₂O (35 mL) was added to the mixture. The aqueous phase was extracted with EtOAc (30 mL×3). The combined organic layers were washed with brine (30 mL), dried over anhydrous Na₂SO₄, filtered and concentrated. The residue was purified by prep-TLC (DCM:MeOH=20:1) to give (R)-3-(3-(4-((7-(4-chloro-1,3,5-triazin-2-yl)-2,7-diazaspiro[4.4]nonan-2-yl)methyl)phenyl)-5-phenyl-3H-imidazo[4,5-b]pyridin-2-yl)pyridin-2-amine (90.0 mg, yield: 39%) as yellow oil. MS: m/z=615.3 [M+H]⁺.

### Step 2: (R)-4-(7-(4-(2-(2-Aminopyridin-3-yl)-5-phenyl-3H-imidazo[4,5-b]pyridin-3-yl)benzyl)-2,7-diazaspiro[4.4]nonan-2-yl)-1,3,5-triazine-2-carbonitrile

To a solution of (R)-3-(3-(4-((7-(4-chloro-1,3,5-triazin-2-yl)-2,7-diazaspiro[4.4]nonan-2-yl)methyl)phenyl)-5-phenyl-3H-imidazo[4,5-b]pyridin-2-yl)pyridin-2-amine (90.0 mg, 146 μmol) in DMSO (1 mL) were added KCN (10 mg, 154 μmol) and DABCO (3.28 mg, 29.3 μmol) at 25° C. This mixture was stirred at 25° C. for 16 hr and then H₂O (35 mL) was added to the mixture. The aqueous phase was extracted with EtOAc (30 mL×3). The combined organic phase was washed with brine (30 mL), dried over anhydrous Na₂SO₄, filtered and concentrated. The residue was purified by prep-TLC (CH₂Cl₂:MeOH=20:1) to give (R)-4-(7-(4-(2-(2-aminopyridin-3-yl)-5-phenyl-3H-imidazo[4,5-b]pyridin-3-yl)benzyl)-2,7-diazaspiro[4.4]nonan-2-yl)-1,3,5-triazine-2-carbonitrile (Example 255, 6.30 mg, yield: 7.0%) as a light-yellow solid. MS: m/z=606.3 [M+H]⁺. ¹H NMR (400 MHz, Methanol-d₄) δ=8.54 (d, J=4.0 Hz, 1H), 8.18 (d, J=8.4 Hz, 1H), 8.05-7.99 (m, 2H), 7.98-7.91 (m, 2H), 7.58-7.51 (m, 2H), 7.49-7.32 (m, 5H), 7.33-7.28 (m, 1H), 6.44 (dd, J=7.2, 4.8 Hz, 1H), 3.77 (d, J=2.8 Hz, 2H), 3.69-3.52 (m, 4H), 2.86-2.58 (m, 4H), 2.10-1.99 (m, 2H), 1.96-1.87 (m, 2H).

### Example 256: 4-((2R,6R)-4-(4-(2-(2-Aminopyridin-3-yl)-5-phenyl-3H-imidazo[4,5-b]pyridin-3-yl)benzyl)-2,6-dimethylpiperazin-1-yl)-1,3,5-triazine-2-carbonitrile

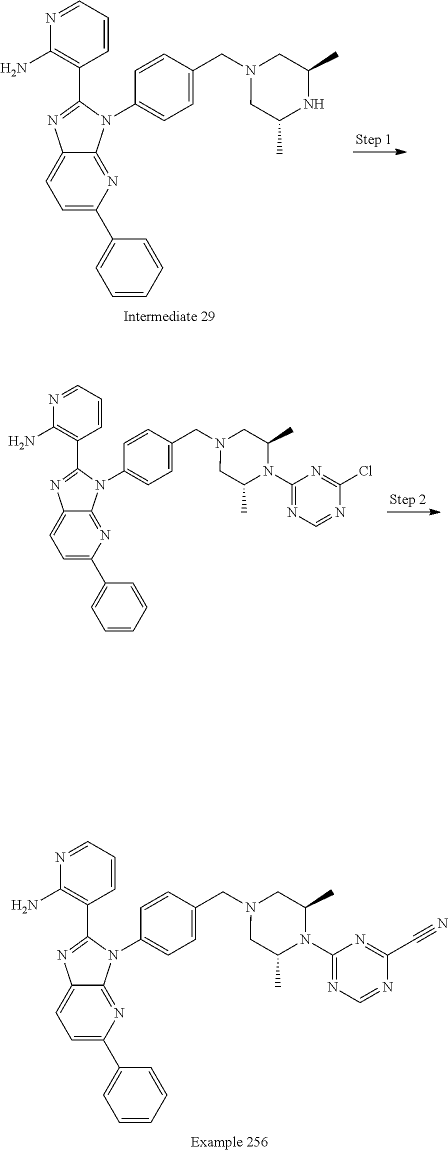 [SMILES: C[C@@H]1CN(Cc2ccc(-n3c(-c4cccnc4N)nc4ccc(-c5ccccc5)nc43)cc2)C[C@@H](C)N1 ; C[C@@H]1CN(Cc2ccc(-n3c(-c4cccnc4N)nc4ccc(-c5ccccc5)nc43)cc2)C[C@@H](C)N1c1ncnc(Cl)n1 ; C[C@@H]1CN(Cc2ccc(-n3c(-c4cccnc4N)nc4ccc(-c5ccccc5)nc43)cc2)C[C@@H](C)N1c1ncnc(C#N)n1]

### Step 1: 3-(3-(4-(((3R,5R)-4-(4-Chloro-1,3,5-triazin-2-yl)-3,5-dimethylpiperazin-1-yl)methyl)phenyl)-5-phenyl-3H-imidazo[4,5-b]pyridin-2-yl)pyridin-2-amine

To a solution of Intermediate 29 (100 mg, 204 μmol) and DIEA (66.0 mg, 511 μmol) in THF (6 mL) and DMF (6 mL) was added 2,4-dichloro-1,3,5-triazine (33.7 mg, 225 μmol). The mixture was stirred at 0° C. for 1 hr. The mixture was partitioned between EtOAc (50 mL) and H₂O (30 mL). The separated organic layer was washed with H₂O (25 mL×3) and dried over Na₂SO₄, filtered, and concentrated. The residue was purified by prep-TLC (CH₂Cl₂:MeOH=15:1) to give 3-(3-(4-(((3R,5R)-4-(4-chloro-1,3,5-triazin-2-yl)-3,5-dimethylpiperazin-1-yl)methyl)phenyl)-5-phenyl-3H-imidazo[4,5-b]pyridin-2-yl)pyridin-2-amine (36.5 mg, yield: 30%) as a yellow solid. MS: m/z=603.3 [M+H]⁺. ¹H NMR (400 MHz, Methanol-d₄) δ 8.39 (s, 1H), 8.19 (d, J=8.4 Hz, 1H), 8.06-7.91 (m, 4H), 7.57 (d, J=8.4 Hz, 2H), 7.48-7.32 (m, 6H), 6.48 (dd, J=7.6, 5.2 Hz, 1H), 4.50-4.44 (m, 2H), 3.98-3.79 (m, 2H), 3.19-3.10 (m, 2H), 2.78-2.70 (m, 2H), 1.43 (d, J=6.4 Hz, 6H).

### Step 2: 4-((2R,6R)-4-(4-(2-(2-Aminopyridin-3-yl)-5-phenyl-3H-imidazo[4,5-b]pyridin-3-yl)benzyl)-2,6-dimethylpiperazin-1-yl)-1,3,5-triazine-2-carbonitrile

To a solution of 3-(3-(4-(((3R,5R)-4-(4-chloro-1,3,5-triazin-2-yl)-3,5-dimethylpiperazin-1-yl)methyl)phenyl)-5-phenyl-3H-imidazo[4,5-b]pyridin-2-yl)pyridin-2-amine (60 mg, 99.5 μmol) in DMSO (1 mL) were added KCN (13.0 mg, 199 μmol) and DABCO (1.12 mg, 9.95 μmol). The mixture was stirred at 25° C. for 16 hr. The mixture was quenched with H₂O (10 mL) and extracted with EtOAc (10 mL×3). The combined organic layers were dried over anhydrous Na₂SO₄, filtered and concentrated. The residue was purified by prep-HPLC (column: Waters xbridge 150×25 mm 10 μm; mobile phase: [water (NH₄HCO₃)-ACN]; B %: 65%-95%, 10 min) to give 4-((2R,6R)-4-(4-(2-(2-aminopyridin-3-yl)-5-phenyl-3H-imidazo[4,5-b]pyridin-3-yl)benzyl)-2,6-dimethylpiperazin-1-yl)-1,3,5-triazine-2-carbonitrile (Example 256, 8.8 mg, yield: 15%) as a yellow lyophilized powder. MS: m/z=594.3 [M+H]⁺. ¹H NMR (400 MHz, Dimethylsulfoxide-d₆) δ 8.76 (s, 1H), 8.27 (d, J=8.4 Hz, 1H), 8.08-7.95 (m, 4H), 7.57-7.36 (m, 7H), 7.18 (dd, J=7.6, 1.6 Hz, 1H), 7.03 (br s, 2H), 6.39 (dd, J=7.6, 4.8 Hz, 1H), 4.40-4.33 (m, 2H), 4.00-3.79 (m, 2H), 3.18-3.09 (m, 2H), 2.77-2.68 (m, 2H), 1.35 (d, J=6.4 Hz, 6H).

### Example 257: 4-(5-(4-(2-(2-Aminopyridin-3-yl)-5-phenyl-3H-imidazo[4,5-b]pyridin-3-yl)benzyl)-2,5-diazabicyclo[2.2.2]octan-2-yl)-1,3,5-triazine-2-carbonitrile

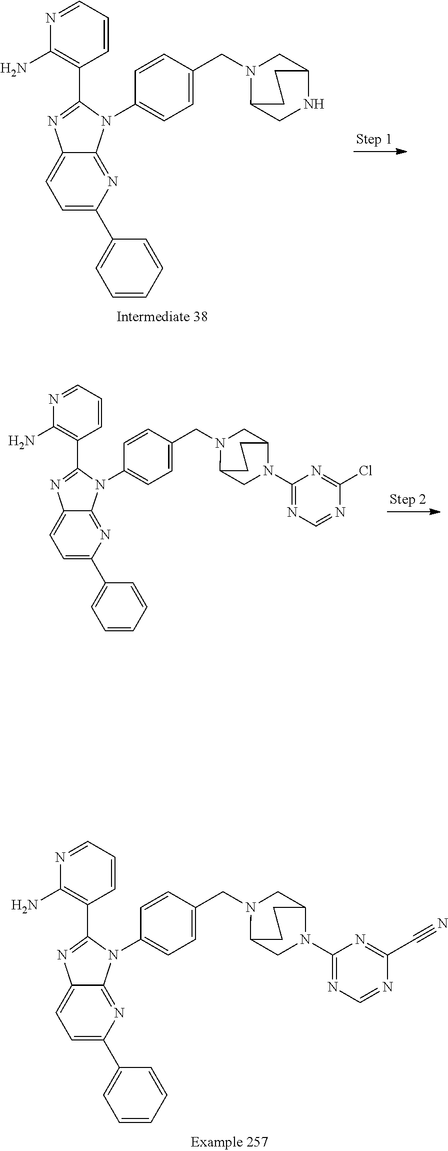 [SMILES: Nc1ncccc1-c1nc2ccc(-c3ccccc3)nc2n1-c1ccc(CN2CC3CCC2CN3)cc1 ; Nc1ncccc1-c1nc2ccc(-c3ccccc3)nc2n1-c1ccc(CN2CC3CCC2CN3c2ncnc(Cl)n2)cc1 ; N#Cc1ncnc(N2CC3CCC2CN3Cc2ccc(-n3c(-c4cccnc4N)nc4ccc(-c5ccccc5)nc43)cc2)n1]

### Step 1: 3-(3-(4-((5-(4-Chloro-1,3,5-triazin-2-yl)-2,5-diazabicyclo[2.2.2]octan-2-yl)methyl)phenyl)-5-phenyl-3H-imidazo[4,5-b]pyridin-2-yl)pyridin-2-amine

To a solution of Intermediate 38 (100 mg, 205 μmol) and 2,4-dichloro-1,3,5-triazine (33.8 mg, 226 μmol) in THF (5 mL) was added DLEA (133 mg, 1.0 mmol). The mixture was stirred at 0° C. for 1 hr. The reaction mixture was quenched with H₂O (10 mL) at 25° C., then mixture was extracted with CH₂Cl₂ (10 mL×3). The combined organic layers were washed with brine (30 mL), dried over Na₂SO₄, filtered, and concentrated under reduced pressure. The crude 3-(3-(4-((5-(4-chloro-1,3,5-triazin-2-yl)-2,5-diazabicyclo[2.2.2]octan-2-yl)methyl)phenyl)-5-phenyl-3H-imidazo[4,5-b]pyridin-2-yl)pyridin-2-amine (120 mg, yield: 78%) was obtained as a yellow solid. MS: m/z=601.4 [M+H]⁺.

### Step 2: 4-(5-(4-(2-(2-Aminopyridin-3-yl)-5-phenyl-3H-imidazo[4,5-b]pyridin-3-yl)benzyl)-2,5-diazabicyclo[2.2.2]octan-2-yl)-1,3,5-triazine-2-carbonitrile

To a solution of 3-(3-(4-((5-(4-chloro-1,3,5-triazin-2-yl)-2,5-diazabicyclo[2.2.2]octan-2-yl)methyl)phenyl)-5-phenyl-3H-imidazo[4,5-b]pyridin-2-yl)pyridin-2-amine (50 mg, 83.2 μmol) in DMSO (1 mL) were added KCN (16.3 mg, 250 μmol) and DABCO (1.87 mg, 16.6 μmol). The mixture was stirred at 25° C. for 16 hr. The mixture was quenched with H₂O (10 mL) and extracted with ethyl acetate (10 mL×3). The combined organic layers were dried over anhydrous Na₂SO₄, filtered and concentrated. The residue was purified by prep-TLC (MeOH:CH₂Cl₂=1:10) to give 4-(5-(4-(2-(2-aminopyridin-3-yl)-5-phenyl-3H-imidazo[4,5-b]pyridin-3-yl)benzyl)-2,5-diazabicyclo[2.2.2]octan-2-yl)-1,3,5-triazine-2-carbonitrile (Example 257, 9.5 mg, yield: 18%) as a yellow solid. MS: m/z=592.2 [M+H]⁺. ¹H NMR (400 MHz, Acetonitrile-d₃) δ 8.52-8.39 (m, 1H), 8.17 (d, J=8.4 Hz, 1H), 8.04-7.97 (m, 3H), 7.89 (d, J=8.4 Hz, 1H), 7.54 (d, J=8.4 Hz, 2H), 7.50-7.35 (m, 5H), 7.15 (d, J=6.0 Hz, 1H), 6.57 (br s, 2H), 6.38 (dd, J=7.6, 4.8 Hz, 1H), 4.76-4.62 (m, 1H), 3.95-3.81 (m, 3H), 3.58-3.46 (m, 1H), 3.08-3.04 (m, 1H), 3.06 (s, 1H), 1.78-1.66 (m, 2H), 1.30-1.24 (m, 2H).

### Example 258: 4-(4-(4-(2-(2-Aminopyridin-3-yl)-5-cyclopropyl-3H-imidazo[4,5-b]pyridin-3-yl)benzyl)piperazin-1-yl)pyrimidine-2-carbonitrile

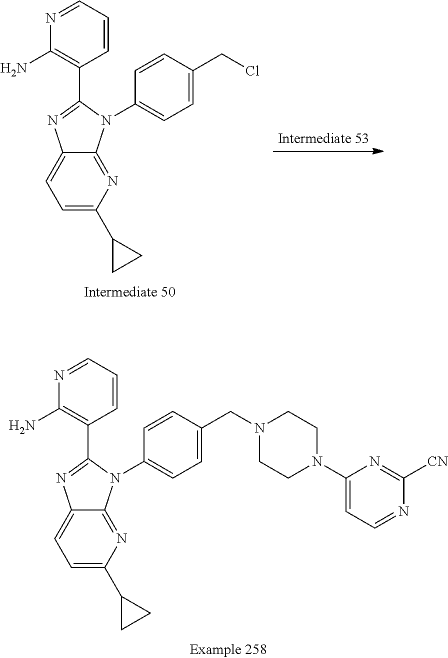 [SMILES: Nc1ncccc1-c1nc2ccc(C3CC3)nc2n1-c1ccc(CCl)cc1 ; N#Cc1nccc(N2CCN(Cc3ccc(-n4c(-c5cccnc5N)nc5ccc(C6CC6)nc54)cc3)CC2)n1]

To a solution of Intermediate 53 (147 mg, 485 μmol, TFA salt) and Intermediate 50 (200 mg, 485 μmol, HCl salt) in DMF (5 mL) were added NaI (36.3 mg, 242 μmol) and K₂CO₃ (335 mg, 2.43 mmol). The mixture was stirred at 50° C. for 2 hr. The reaction mixture was diluted with H₂O (20 mL) and extracted with EtOAc (30 mL×3). The combined organic layers were washed with brine (30 mL), dried over Na₂SO₄, filtered, and concentrated under reduced pressure. The residue was purified by silica gel flash chromatography (Eluent of 0˜7% MeOH in CH₂Cl₂) to give 4-(4-(4-(2-(2-aminopyridin-3-yl)-5-cyclopropyl-3H-imidazo[4,5-b]pyridin-3-yl)benzyl)piperazin-1-yl)pyrimidine-2-carbonitrile (Example 258, 46.2 mg, yield: 17%) as yellow solid. MS: m/z=529.3 [M+H]⁺. ¹H NMR (400 MHz, Methanol-d₄) δ 8.20 (d, J=6.4 Hz, 1H), 8.01-7.96 (m, 2H), 7.54 (d, J=8.0 Hz, 2H), 7.37 (d, J=8.4 Hz, 2H), 7.30-7.25 (m, 2H), 6.97 (d, J=6.4 Hz, 1H), 6.47 (dd, J=7.6, 5.2 Hz, 1H), 3.85-3.75 (m, 4H), 3.65 (s, 2H), 2.64-2.59 (m, 4H), 2.23-2.15 (m, 1H), 1.00-0.93 (m, 4H).

### Example 259: 4-(4-(4-(2-(2-Aminopyridin-3-yl)-5-(2-oxo-1,2-dihydropyridin-3-yl)-3H-imidazo[4,5-b]pyridin-3-yl)benzyl)piperazin-1-yl)pyrimidine-2-carbonitrile

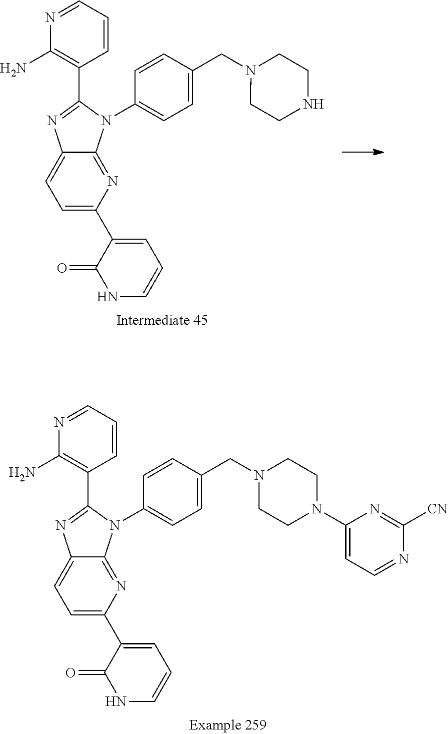 [SMILES: Nc1ncccc1-c1nc2ccc(-c3ccc[nH]c3=O)nc2n1-c1ccc(CN2CCNCC2)cc1 ; N#Cc1nccc(N2CCN(Cc3ccc(-n4c(-c5cccnc5N)nc5ccc(-c6ccc[nH]c6=O)nc54)cc3)CC2)n1]

A mixture of Intermediate 45 (100 mg, 209 μmol), 4-chloropyrimidine-2-carbonitrile (29 mg, 209 μmol) and DIEA (135 mg, 1.04 mmol) in NMP (2 mL) was taken up into a microwave tube. The sealed tube was heated at 130° C. for 1 hr under microwave. The reaction mixture was quenched with H₂O (5 mL) at 0° C., and then extracted with CH₂Cl₂ (15 mL×2). The combined organic layers were washed with brine (15 mL), dried over Na₂SO₄, filtered, and concentrated under reduced pressure. After purified by prep-HPLC (column: Phenomenex C18 150*25 mm*10 μm; mobile phase: [water (NH₄HCO₃)-ACN]; B %: 20%-50%, over 14 min) and prep-TLC (CH₂Cl₂:MeOH=10:1), 4-(4-(4-(2-(2-aminopyridin-3-yl)-5-(2-oxo-1,2-dihydropyridin-3-yl)-3H-imidazo[4,5-b]pyridin-3-yl)benzyl)piperazin-1-yl)pyrimidine-2-carbonitrile (Example 259, 13.8 mg, yield: 11%) was obtained as a brown solid. MS: m/z=582.3 [M+H]⁺. ¹H NMR (400 MHz, Methanol-d₄) δ 8.51 (d, J=8.4 Hz, 1H), 8.35 (dd, J=7.2, 2.0 Hz, 1H), 8.20-8.13 (m, 2H), 7.98 (dd, J=4.8, 1.6 Hz, 1H), 7.56 (d, J=8.4 Hz, 2H), 7.52-7.48 (m, 1H), 7.45 (d, J=8.4 Hz, 2H), 7.32 (dd, J=7.6, 1.6 Hz, 1H), 6.95 (d, J=6.4 Hz, 1H), 6.54-6.49 (m, 1H), 6.47 (dd, J=8.0, 5.2 Hz, 1H), 3.83-3.73 (m, 4H), 3.68 (s, 2H), 2.62-2.57 (m, 4H).

### Example 260: 4-(4-(4-(2-(2-aminopyridin-3-yl)-5-(1-methyl-2-oxo-1,2-dihydropyridin-3-yl)-3H-imidazo[4,5-b]pyridin-3-yl)benzyl)piperazin-1-yl)pyrimidine-2-carbonitrile

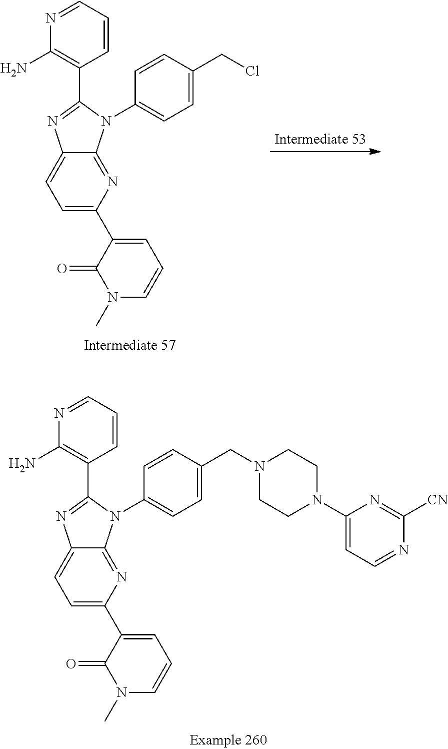 [SMILES: Cn1cccc(-c2ccc3nc(-c4cccnc4N)n(-c4ccc(CCl)cc4)c3n2)c1=O ; Cn1cccc(-c2ccc3nc(-c4cccnc4N)n(-c4ccc(CN5CCN(c6ccnc(C#N)n6)CC5)cc4)c3n2)c1=O]

To a solution of Intermediate 57 (150 mg, 339 μmol) and Intermediate 53 (103 mg, 339 mol, TFA) in DMF (2 mL) were added K₂CO₃ (234 mg, 1.69 mmol) and NaI (10.2 mg, 67.7 μmol). The mixture was stirred at 80° C. for 16 hr. The reaction mixture was quenched with H₂O (5 mL) at 25° C., and then diluted with CH₂Cl₂ (10 mL) and extracted with H₂O (15 mL×2). The combined organic layers were washed with brine (15 mL), dried over Na₂SO₄, filtered and concentrated under reduced pressure. After purified by prep-HPLC (column: Waters xbridge 150*25 mm*10 μm; mobile phase: [water (NH₄HCO₃)-ACN]; gradient: 30%-50% B over 14 min), 4-(4-(4-(2-(2-aminopyridin-3-yl)-5-(1-methyl-2-oxo-1,2-dihydropyridin-3-yl)-3H-imidazo[4,5-b]pyridin-3-yl)benzyl)piperazin-1-yl)pyrimidine-2-carbonitrile (Example 260, 10.6 mg, yield: 5.3%) was obtained as a yellow solid. MS: m/z=596.3 [M+H]⁺. ¹H NMR (400 MHz, Methanol-d₄) δ 8.46 (d, J=8.4 Hz, 1H), 8.27-8.22 (m, 1H), 8.20-8.14 (m, 2H), 8.00-7.96 (m, 1H), 7.74 (dd, J=6.8, 2.4 Hz, 1H), 7.56 (d, J=8.4 Hz, 2H), 7.45 (d, J=8.4 Hz, 2H), 7.32 (dd, J=8.0, 2.0 Hz, 1H), 7.01-6.92 (m, 1H), 6.52-6.42 (m, 2H), 3.83-3.74 (m, 4H), 3.70-3.67 (m, 5H), 2.62-2.57 (m, 4H).

### Example 261: 4-((1-(4-(2-(2-Aminopyridin-3-yl)-5-(1-methyl-2-oxo-1,2-dihydropyridin-3-yl)-3H-imidazo[4,5-b]pyridin-3-yl)benzyl)piperidin-4-yl)amino)pyrimidine-2-carbonitrile

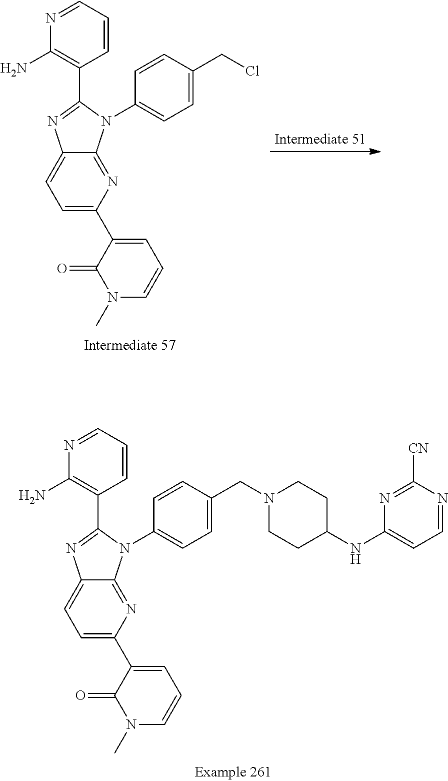 [SMILES: Cn1cccc(-c2ccc3nc(-c4cccnc4N)n(-c4ccc(CCl)cc4)c3n2)c1=O ; Cn1cccc(-c2ccc3nc(-c4cccnc4N)n(-c4ccc(CN5CCC(Nc6ccnc(C#N)n6)CC5)cc4)c3n2)c1=O]

To a solution of Intermediate 57 (160 mg, 361 μmol) and Intermediate 51 (88.1 mg, 278 μmol) in DMF (5 mL) were added NaI (10.8 mg, 72.3 μmol) and K₂CO₃ (299 mg, 2.17 mmol). The mixture was stirred at 50° C. for 2 hr. The reaction mixture was diluted with H₂O (50 mL) and extracted with EtOAc (50 mL×2). The combined organic layers were washed with brine (50 mL×2), dried over Na₂SO₄, filtered, and concentrated under reduced pressure. After purified by silica gel flash chromatography (Eluent of 0˜10% MeOH in CH₂Cl₂) and then purified by prep-HPLC (column: Waters Xbridge 150×25 mm×5 um; mobile phase: [water (NH₄HCO₃)-ACN]; gradient: 22%-52% B over 10 min), 4-((1-(4-(2-(2-aminopyridin-3-yl)-5-(1-methyl-2-oxo-1,2-dihydropyridin-3-yl)-3H-imidazo[4,5-b]pyridin-3-yl)benzyl)piperidin-4-yl)amino)pyrimidine-2-carbonitrile (Example 261, 54.5 mg, yield: 24%) was obtained as a light-yellow lyophilized powder. MS: m/z=610.4 [M+H]⁺. ¹H NMR (400 MHz, Dimethylsulfoxide-d₆) δ 8.64 (d, J=8.4 Hz, J H), 8.21-8.17 (m, 2H), 8.09-8.05 (m, 2H), 7.99 (dd, J=4.8, 1.6 Hz, 1H), 7.85 (dd, J=6.8, 2.0 Hz, 1H), 7.50-7.48 (m, 4H), 7.13 (dd, J=7.6, 1.6 Hz, 1H), 7.04 (br s, 2H), 6.67 (d, J=6.0 Hz, 1H), 6.40-6.35 (m, 2H), 3.88-3.76 (m, 1H), 3.59 (s, 2H), 3.55 (s, 3H), 2.85-2.80 (m, 2H), 2.19-2.10 (m, 2H), 1.98-1.83 (m, 2H), 1.55-1.43 (m, 2H)

### Example 262: 4-((1-(4-(2-(2-Aminopyridin-3-yl)-5-cyclobutyl-3H-imidazo[4,5-b]pyridin-3-yl)benzyl)piperidin-4-yl)amino)pyrimidine-2-carbonitrile

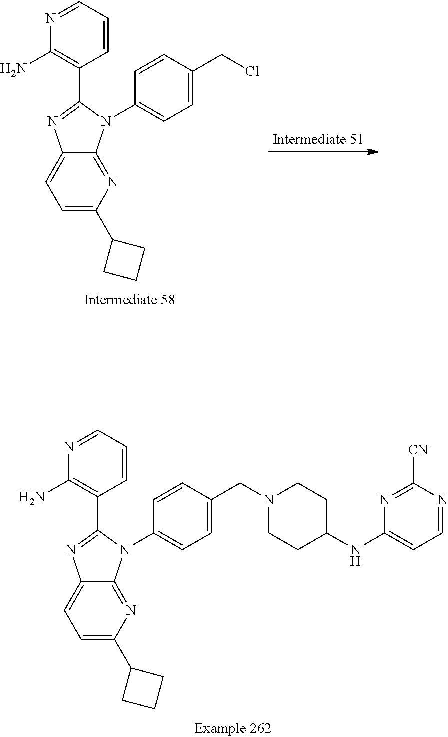 [SMILES: Nc1ncccc1-c1nc2ccc(C3CCC3)nc2n1-c1ccc(CCl)cc1 ; N#Cc1nccc(NC2CCN(Cc3ccc(-n4c(-c5cccnc5N)nc5ccc(C6CCC6)nc54)cc3)CC2)n1]

To a solution of Intermediate 58 (281 mg, 659 μmol, HCl salt) and Intermediate 51 (209 mg, 659 μmol, TFA) in DMF (5 mL) were added K₂CO₃ (273 mg, 1.98 mmol) and NaI (19.7 mg, 132 μmol). The mixture was stirred at 60° C. for 1 hr. The mixture was filtered, and the filtrate was concentrated to give the crude product. After purified by prep-HPLC (column: Waters xbridge 150*25 mm*10 μm; mobile phase: [water (NH₄HCO₃)-ACN]; gradient: 37%-67% B over 14 min), 4-((1-(4-(2-(2-aminopyridin-3-yl)-5-cyclobutyl-3H-imidazo[4,5-b]pyridin-3-yl)benzyl)piperidin-4-yl)amino)pyrimidine-2-carbonitrile (Example 262, 67.7 mg, yield: 18%) was obtained as an off-white lyophilized powder. MS: m/z=557.4 [M+H]⁺. ¹H NMR (400 MHz, Dimethylsulfoxide-d₆) δ 8.25-8.02 (m, 3H), 7.97 (dd, J=4.8, 2.0 Hz, 1H), 7.45 (d, J=8.4 Hz, 2H), 7.30 (d, J=8.4 Hz, 2H), 7.30 (d, J=8.4 Hz, 1H), 7.09 (dd, J=7.6, 1.6 Hz, 1H), 6.97 (br s, 2H), 6.67 (d, J=6.0 Hz, 1H), 6.35 (dd, J=7.6, 4.8 Hz, 1H), 3.86-3.76 (m, 1H), 3.74-3.64 (m, 1H), 3.58 (s, 2H), 2.87-2.75 (m, 2H), 2.28-2.09 (m, 6H), 1.99-1.74 (m, 4H), 1.56-1.42 (m, 2H).

### Example 263: 4-((1-(4-(2-(2-Aminopyridin-3-yl)-5-(cyclohex-1-en-1-yl)-3H-imidazo[4,5-b]pyridin-3-yl)benzyl)piperidin-4-yl)amino)pyrimidine-2-carbonitrile

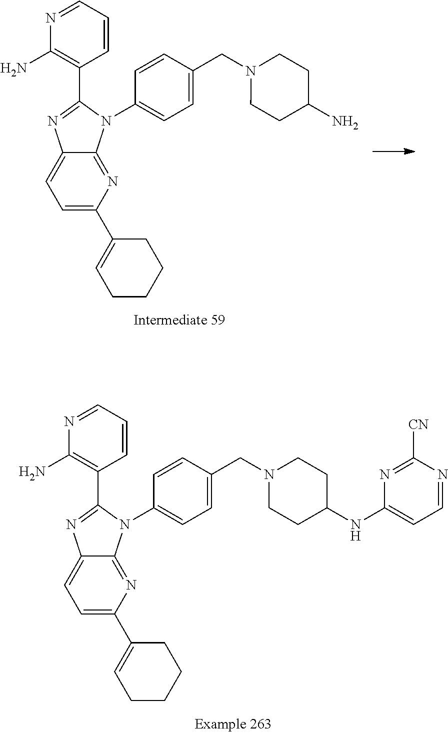 [SMILES: Nc1ncccc1-c1nc2ccc(C3=CCCCC3)nc2n1-c1ccc(CN2CCC(N)CC2)cc1 ; N#Cc1nccc(NC2CCN(Cc3ccc(-n4c(-c5cccnc5N)nc5ccc(C6=CCCCC6)nc54)cc3)CC2)n1]

A mixture of Intermediate 59 (80 mg, 167 μmol), 4-chloropyrimidine-2-carbonitrile (25.6 mg, 183 μmol) and DIEA (65 mg, 500 μmol) in NMP (2 mL) was taken up into a microwave tube. The sealed tube was stirred at 130° C. for 0.5 hr under microwave. The reaction mixture was poured into H₂O (10 mL), extracted with CH₂Cl₂ (15 mL). The organic layer was dried over anhydrous Na₂SO₄, filtered and concentrated under reduced pressure. The residue was purified by prep-HPLC (column: Phenomenex C18 150*25 mm*10 μm; mobile phase: [water (NH₄HCO₃)-ACN]; gradient: 45%-75% B over 14 min) to give 4-((1-(4-(2-(2-Aminopyridin-3-yl)-5-(cyclohex-1-en-1-yl)-3H-imidazo[4,5-b]pyridin-3-yl)benzyl)piperidin-4-yl)amino)pyrimidine-2-carbonitrile (Example 263, 17.3 mg, yield: 18%) as a brown lyophilized powder. MS: m/z=583.4 [M+H]⁺. ¹H NMR (400 MHz, Dimethylsulfoxide-d₆) δ 8.22-8.01 (m, 3H), 7.97 (dd, J=4.8, 2.0 Hz, 1H), 7.52 (d, J=8.4 Hz, 1H), 7.48-7.41 (m, 2H), 7.40-7.33 (m, 2H), 7.97 (dd, J=8.0, 2.0 Hz, 1H), 7.06-6.95 (m, 3H), 6.76-6.53 (m, 2H), 6.34 (dd, J=8.0, 5.2 Hz, 1H), 3.91-3.69 (m, 1H), 3.61 (s, 2H), 2.83-2.77 (m, 2H), 2.46-2.42 (m, 2H), 2.22-2.09 (m, 4H), 1.89-1.87 (m, 2H), 1.72-1.64 (m, 2H), 1.62-1.54 (m, 2H), 1.53-1.38 (m, 2H).

### Example 264: 4-((1-(4-(2-(2-Aminopyridin-3-yl)-5-cyclohexyl-3H-imidazo[4,5-b]pyridin-3-yl)benzyl)piperidin-4-yl)amino)pyrimidine-2-carbonitrile

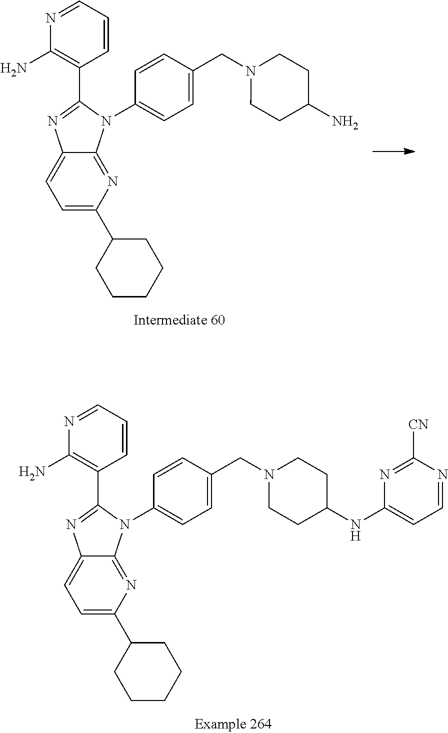 [SMILES: Nc1ncccc1-c1nc2ccc(C3CCCCC3)nc2n1-c1ccc(CN2CCC(N)CC2)cc1 ; N#Cc1nccc(NC2CCN(Cc3ccc(-n4c(-c5cccnc5N)nc5ccc(C6CCCCC6)nc54)cc3)CC2)n1]

A mixture of Intermediate 60 (50 mg, 104 μmol), 4-chloropyrimidine-2-carbonitrile (15.9 mg, 114 μmol) and DIEA (40 mg, 311 μmol) in NMP (2 mL) was taken up into a microwave tube. The sealed tube was stirred at 130° C. for 0.5 hr under microwave. The reaction mixture was poured into H₂O (10 mL), extracted with CH₂Cl₂ (15 mL×3). The organic phase was dried over anhydrous Na₂SO₄, filtered and concentrated under reduced pressure. The residue was purified by prep-HPLC (column: Phenomenex C18 150*25 mm*10 μm; mobile phase: [water (NH₄HCO₃)-ACN]; gradient: 47%-77% B over 14 min) to give 4-((1-(4-(2-(2-Aminopyridin-3-yl)-5-cyclohexyl-3H-imidazo[4,5-b]pyridin-3-yl)benzyl)piperidin-4-yl)amino)pyrimidine-2-carbonitrile (Example 264, 4.8 mg, yield: 8%) as an off-white lyophilized powder. MS: m/z=585.4. [M+H]⁺. ¹H NMR (400 MHz, Methanol-d₄) δ 8.03 (d, J=8.0 Hz, 1H), 8.01-7.96 (m, 1H), 7.94 (dd, J=5.2, 2.0 Hz, 1H), 7.50 (d, J=8.4 Hz, 2H), 7.37 (d, J=8.4 Hz, 2H), 7.33-7.22 (m, 2H), 6.68-6.57 (m, 1H), 6.44 (dd, J=7.6, 4.8 Hz, 1H), 4.06-3.85 (m, 1H), 3.64 (s, 2H), 2.95 (d, J=11.6 Hz, 2H), 2.81-2.73 (m, 1H), 2.31-2.19 (m, 2H), 2.03-1.96 (m, 2H), 1.93-1.87 (m, 2H), 1.86-1.79 (m, 2H), 1.77-1.68 (m, 1H), 1.66-1.51 (m, 4H), 1.46-1.35 (m, 2H), 1.33-1.24 (m, 1H).

### Example 265: 4-((1-(4-(2-(2-Aminopyridin-3-yl)-5-(3-cyanophenyl)-3H-imidazo[4,5-b]pyridin-3-yl)benzyl)piperidin-4-yl)amino)pyrimidine-2-carbonitrile

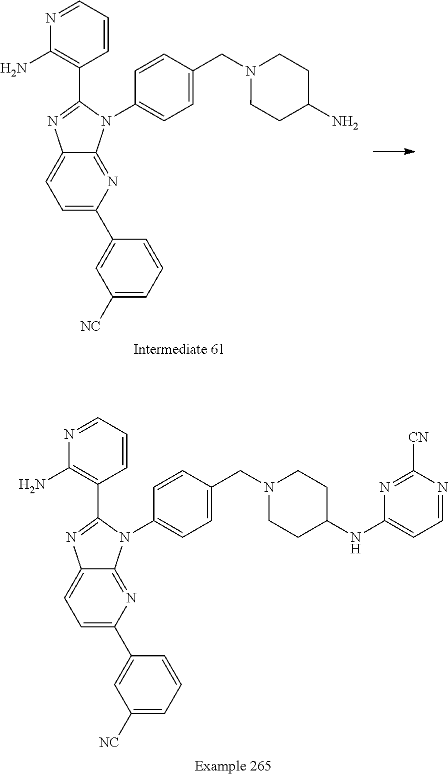 [SMILES: N#Cc1cccc(-c2ccc3nc(-c4cccnc4N)n(-c4ccc(CN5CCC(N)CC5)cc4)c3n2)c1 ; N#Cc1cccc(-c2ccc3nc(-c4cccnc4N)n(-c4ccc(CN5CCC(Nc6ccnc(C#N)n6)CC5)cc4)c3n2)c1]

A mixture of Intermediate 61 (60 mg, 200 μmol), 4-chloropyrimidine-2-carbonitrile (18.4 mg, 132 μmol) and DIEA (47 mg, 360 μmol) in NMP (2 mL) was taken up into a microwave tube. The sealed tube was stirred at 130° C. for 0.5 hr under microwave. The reaction mixture was poured into H₂O (10 mL), extracted with CH₂Cl₂ (15 mL×3). The organic phase was dried over anhydrous Na₂SO₄, filtered and concentrated under reduced pressure. The residue was purified by prep-HPLC (column: Phenomenex C18 150*25 mm*10 μm; mobile phase: [water (NH₄HCO₃)-ACN]; gradient: 40%-70% B over 14 min) to give 4-((1-(4-(2-(2-Aminopyridin-3-yl)-5-(3-cyanophenyl)-3H-imidazo[4,5-b]pyridin-3-yl)benzyl)piperidin-4-yl)amino)pyrimidine-2-carbonitrile (Example 265, 5.9 mg, yield: 8%) as an off-white lyophilized powder. MS: m/z=604.4 [M+H]⁺. ¹H NMR (400 MHz, Methanol-d₄) δ 8.43-8.29 (m, 2H), 8.23 (d, J=8.4 Hz, 1H), 8.04-7.93 (m, 3H), 7.74-7.68 (m, 1H), 7.63-7.54 (m, 3H), 7.46 (d, J=8.4 Hz, 2H), 7.33 (dd, J=7.6, 1.6 Hz, 1H), 6.67-6.51 (m, 1H), 6.47 (dd, J=7.6, 4.8 Hz, 1H), 4.09-3.86 (m, 1H), 3.66 (s, 2H), 3.01-2.92 (m, 2H), 2.32-2.23 (m, 2H), 2.03-1.98 (m, 2H), 1.66-1.55 (m, 2H).

### Example 266: 4-((1-(4-(2-(2-Aminopyridin-3-yl)-5-(6-oxo-1,6-dihydropyridin-3-yl)-3H-imidazo[4,5-b]pyridin-3-yl)benzyl)piperidin-4-yl)amino)pyrimidine-2-carbonitrile

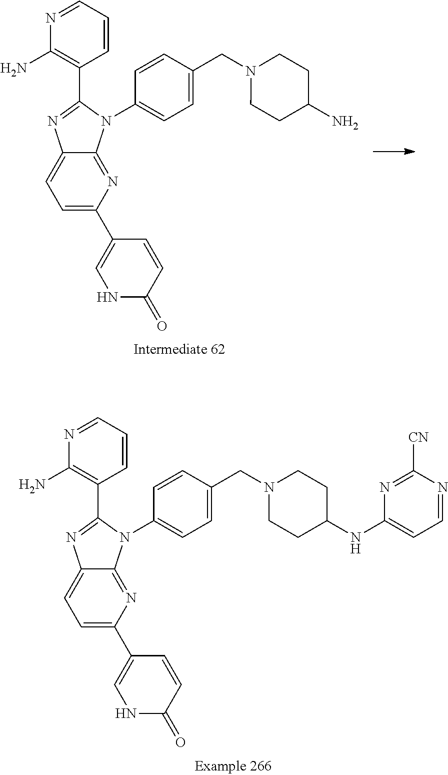 [SMILES: Nc1ncccc1-c1nc2ccc(-c3ccc(=O)[nH]c3)nc2n1-c1ccc(CN2CCC(N)CC2)cc1 ; N#Cc1nccc(NC2CCN(Cc3ccc(-n4c(-c5cccnc5N)nc5ccc(-c6ccc(=O)[nH]c6)nc54)cc3)CC2)n1]

A mixture of Intermediate 62 (100 mg, 203 μmol), 4-chloropyrimidine-2-carbonitrile (31 mg, 223 μmol) and DIEA (79 mg, 609 μmol) in NMP (2 mL) was taken up into a microwave tube. The sealed tube was stirred at 130° C. for 30 min under microwave. The reaction mixture was filtered. The residue was purified by prep-HPLC (column: Waters xbridge 150*25 mm 10 μm; mobile phase: [water (NH₄HCO₃)-ACN]; B %: 26%-56%, 14 min) to give 4-((1-(4-(2-(2-aminopyridin-3-yl)-5-(6-oxo-1,6-dihydropyridin-3-yl)-3H-imidazo[4,5-b]pyridin-3-yl)benzyl)piperidin-4-yl)amino)pyrimidine-2-carbonitrile (Example 266, 1.2 mg, yield. 1%,) as a light-yellow solid. MS: m/z=596.4 [M+H]⁺. ¹H NMR (400 MHz, Dimethylsulfoxide-d₆) δ 11.92-11.76 (m, 1H), 8.20 (d, J=8.4 Hz, 1H), 8.13 (dd, J=9.2, 2.4 Hz, 1H), 8.10-8.03 (m, 3H), 7.99 (dd, J=4.8, 1.6 Hz, 1H), 7.83 (d, J=8.4 Hz, 1H), 7.49-7.46 (m, 2H), 7.44-7.40 (m, 2H), 7.11 (d, J=6.0 Hz, 1H), 7.04 (br s, 2H), 6.70-6.66 (m, 1H), 6.43 (d, J=9.6 Hz, 1H), 6.36 (dd, J=7.6, 4.4 Hz, 1H), 3.86-3.78 (m, 1H), 3.58 (s, 2H), 2.85-2.81 (m, 2H), 2.20-2.12 (m, 2H), 1.94-1.86 (m, 2H), 1.54-1.45 (m, 2H).

### Example 267: 4-((1-(4-(2-(2-Aminopyridin-3-yl)-5-(6-oxo-1,6-dihydropyridin-2-yl)-3H-imidazo[4,5-b]pyridin-3-yl)benzyl)piperidin-4-yl)amino)pyrimidine-2-carbonitrile

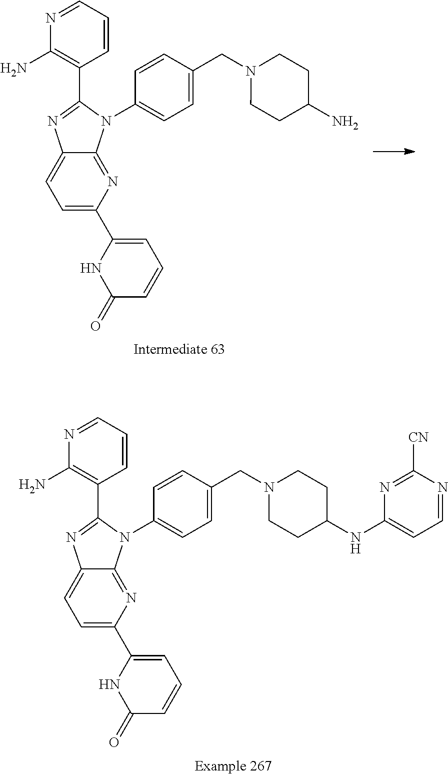 [SMILES: Nc1ncccc1-c1nc2ccc(-c3cccc(=O)[nH]3)nc2n1-c1ccc(CN2CCC(N)CC2)cc1 ; N#Cc1nccc(NC2CCN(Cc3ccc(-n4c(-c5cccnc5N)nc5ccc(-c6cccc(=O)[nH]6)nc54)cc3)CC2)n1]

To a solution of Intermediate 63 (80 mg, 162 pmo) and 4-chloropyrimidine-2-carbonitrile (23 mg, 162 μmol) in ACN (3 mL) were added NaI (2.4 mg, 16 μmol) and K₂CO₃ (67 mg, 487 μmol). The mixture was stirred at 80° C. for 2 hr. The reaction mixture was quenched with H₂O (10 mL) at 25° C., and extracted with EtOAc (15 mL×2). The combined organic layers were washed with brine (20 mL), dried over Na₂SO₄, filtered and concentrated under reduced pressure. The residue was purified by prep-HPLC (column: Waters xbridge 150*25 mm 10 μm; mobile phase: [water (NH₄HCO₃)-ACN]; B %: 22%-52%, 18 min) to give 4-((1-(4-(2-(2-aminopyridin-3-yl)-5-(6-oxo-1,6-dihydropyridin-2-yl)-3H-imidazo[4,5-b]pyridin-3-yl)benzyl)piperidin-4-yl)amino)pyrimidine-2-carbonitrile (Example 267, 15.5 mg, yield: 16%) as a yellow solid. MS: m/z=596.1 [M+H]⁺. ¹H NMR (400 MHz, Dimethylsulfoxide-d₆) δ 10.77 (br s, 1H), 8.33 (d, J=8.4 Hz, 1H), 8.21-8.13 (m, 1H), 8.10-8.00 (m, 3H), 7.67-7.42 (m, 6H), 7.16 d, J=6.8 Hz, 1H), 7.03 (br s, 2H), 6.67 (d, J=5.6 Hz, 1H), 6.50-6.42 (m, 1H), 6.39 (dd, J=7.6, 4.8 Hz, 1H), 3.88-3.76 (m, 1H), 3.60 (s, 2H), 2.86-2.80 (m, 2H), 2.22-2.12 (m, 2H), 1.93-1.85 (m, 2H), 1.56-1.45 (m, 2H).

### Example 268: 4-(2-(4-(2-(2-Aminopyridin-3-yl)-5-phenyl-3H-imidazo[4,5-b]pyridin-3-yl)benzyl)-2,7-diazaspiro[3.5]nonan-7-yl)-1,3,5-triazine-2-carbonitrile

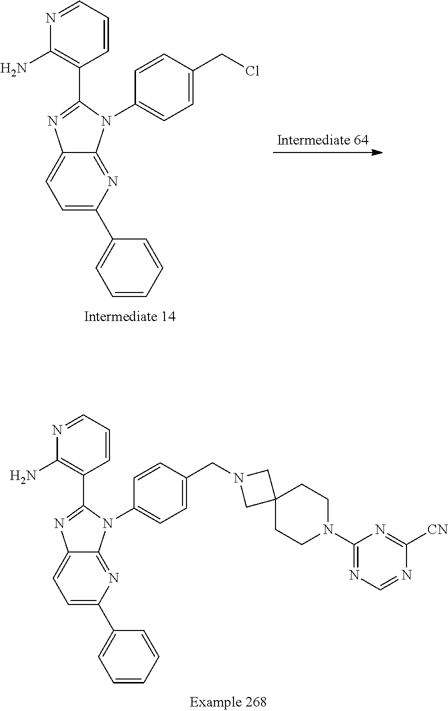 [SMILES: Nc1ncccc1-c1nc2ccc(-c3ccccc3)nc2n1-c1ccc(CCl)cc1 ; N#Cc1ncnc(N2CCC3(CC2)CN(Cc2ccc(-n4c(-c5cccnc5N)nc5ccc(-c6ccccc6)nc54)cc2)C3)n1]

To a solution of Intermediate 14 (233 mg, 565 μmol) and Intermediate 64 (130 mg, 565 μmol, TFA salt) in DMF (5 mL) were added K₂CO₃ (390 mg, 2.82 mmol) and NaI (8.46 mg, 56.5 μmol). The mixture was stirred at 25° C. for 16 hr. After purified by prep-HPLC (column: Phenomenex C₁₈ 150*25 mm*10 μm; mobile phase: [water (NH₄HCO₃)-ACN]; gradient: 50%-80% B over 10 min), 4-(2-(4-(2-(2-aminopyridin-3-yl)-5-phenyl-3H-imidazo[4,5-b]pyridin-3-yl)benzyl)-2,7-diazaspiro[3.5]nonan-7-yl)-1,3,5-triazine-2-carbonitrile (Example 268, 13.6 mg, yield: 3.8%) was obtained as a white solid. MS: m/z=606.3 [M+H]⁺. ¹H NMR (400 MHz, Acetonitrile-d₃) δ 8.52 (s, 1H), 8.17 (d, J=8.4 Hz, 1H), 8.02-7.99 (m, 3H), 7.89 (d, J=8.4 Hz, 1H), 7.48-7.39 (m, 7H), 7.17-7.12 (m, 1H), 6.55 (br s, 2H), 6.40-6.33 (m, 1H), 3.81-3.73 (m, 6H), 3.11 (s, 4H), 1.82-1.78 (m, 4H).

### Example 269: (S)-4-(7-(4-(2-(2-Aminopyridin-3-yl)-5-phenyl-3H-imidazo[4,5-b]pyridin-3-yl)benzyl)-2,7-diazaspiro[4.4]nonan-2-yl)-1,3,5-triazine-2-carbonitrile

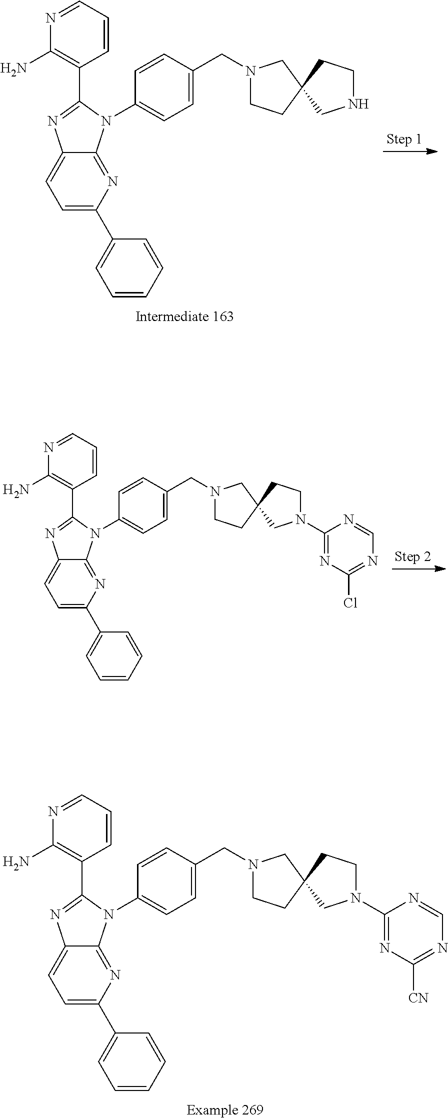 [SMILES: Nc1ncccc1-c1nc2ccc(-c3ccccc3)nc2n1-c1ccc(CN2CC[C@]3(CCNC3)C2)cc1 ; Nc1ncccc1-c1nc2ccc(-c3ccccc3)nc2n1-c1ccc(CN2CC[C@]3(CCN(c4ncnc(Cl)n4)C3)C2)cc1 ; N#Cc1ncnc(N2CC[C@]3(CCN(Cc4ccc(-n5c(-c6cccnc6N)nc6ccc(-c7ccccc7)nc65)cc4)C3)C2)n1]

### Step 1: (S)-3-(3-(4-((7-(4-Chloro-1,3,5-triazin-2-yl)-2,7-diazaspiro[4.4]nonan-2-yl)methyl)phenyl)-5-phenyl-3H-imidazo[4,5-b]pyridin-2-yl)pyridin-2-amine

To a solution of Intermediate 163 (80 mg, 159 μmol) and 2,4-dichloro-1,3,5-triazine (26.3 mg, 175 μmol) in THF (5 mL) and H₂O (1 mL) was added DIEA (103 mg, 797 μmol). The mixture was stirred at 0° C. for 1 hr. The reaction mixture was quenched with H₂O (50 mL) at 25° C. and extracted with CH₂Cl₂ (50 mL×2). The combined organic layers were washed with brine (100 mL), dried over Na₂SO₄, filtered and concentrated under reduced pressure. The crude was purified by silica gel flash chromatography (Eluent of 1˜10% MeOH in CH₂Cl₂) to give (S)-3-(3-(4-((7-(4-chloro-1,3,5-triazin-2-yl)-2,7-diazaspiro[4.4]nonan-2-yl)methyl)phenyl)-5-phenyl-3H-imidazo[4,5-b]pyridin-2-yl)pyridin-2-amine (30 mg, yield: 30%) as a yellow solid. MS: m/z=615.6 [M+H]⁺.

### Step 2: (S)-4-(7-(4-(2-(2-Aminopyridin-3-yl)-5-phenyl-3H-imidazo[4,5-b]pyridin-3-yl)benzyl)-2,7-diazaspiro[4.4]nonan-2-yl)-1,3,5-triazine-2-carbonitrile

To a solution of (S)-3-(3-(4-((7-(4-chloro-1,3,5-triazin-2-yl)-2,7-diazaspiro[4.4]nonan-2-yl)methyl)phenyl)-5-phenyl-3H-imidazo[4,5-b]pyridin-2-yl)pyridin-2-amine (30 mg, 48.8 μmol) and in DMSO (1 mL) were added KCN (6.35 mg, 97.5 μmol) and DABCO (1.09 mg, 9.75 μmol). The mixture was stirred at 25° C. for 16 hr. The mixture was quenched with H₂O (10 mL) and extracted with EtOAc (10 mL×3). The combined organic layers were dried over anhydrous Na₂SO₄, filtered and concentrated. The residue was purified by prep-TLC (CH₂Cl₂:MeOH=10:1) to give (S)-4-(7-(4-(2-(2-aminopyridin-3-yl)-5-phenyl-3H-imidazo[4,5-b]pyridin-3-yl)benzyl)-2,7-diazaspiro[4.4]nonan-2-yl)-1,3,5-triazine-2-carbonitrile (Example 269, 3.3 mg, yield: 11.2%) as a yellow solid. MS: m/z=606.4 [M+H]⁺. ¹H NMR (400 MHz, Chloroform-d) δ 8.54 (d, J=4.8 Hz, 1H), 8.13 (d, J=8.4 Hz, 1H), 8.05 (dd, J=4.8, 1.6 Hz, 1H), 8.01 (d, J=8.0 Hz, 2H), 7.80 (d, J=8.4 Hz, 1H), 7.51-7.37 (m, 7H), 7.11-7.03 (m, 1H), 6.63 (br s, 2H), 6.34 (dd, J=7.6, 5.2 Hz, 1H), 3.74-3.56 (m, 6H), 2.79-2.72 (m, 2H), 2.62-2.57 (m, 2H), 2.10-2.04 (m, 2H), 2.01-1.91 (m, 2H).

### Example 270: 4-(3-(4-(2-(2-Aminopyridin-3-yl)-5-phenyl-3H-imidazo[4,5-b]pyridin-3-yl)benzyl)-3,8-diazabicyclo[3.2.1]octan-8-yl)-1,3,5-triazine-2-carbonitrile

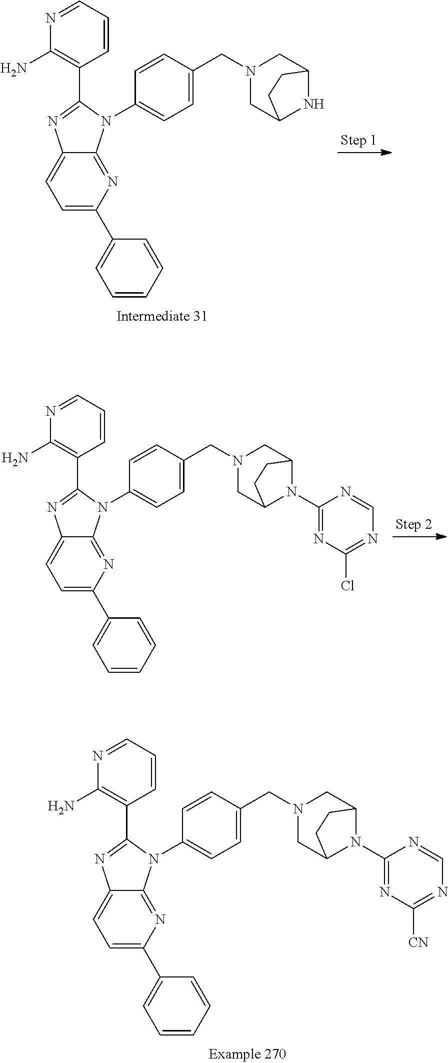 [SMILES: Nc1ncccc1-c1nc2ccc(-c3ccccc3)nc2n1-c1ccc(CN2CC3CCC(C2)N3)cc1 ; Nc1ncccc1-c1nc2ccc(-c3ccccc3)nc2n1-c1ccc(CN2CC3CCC(C2)N3c2ncnc(Cl)n2)cc1 ; N#Cc1ncnc(N2C3CCC2CN(Cc2ccc(-n4c(-c5cccnc5N)nc5ccc(-c6ccccc6)nc54)cc2)C3)n1]

### Step 1: 3-(3-(4-((8-(4-Chloro-1,3,5-triazin-2-yl)-3,8-diazabicyclo[3.2.1]octan-3-yl)methyl)phenyl)-5-phenyl-3H-imidazo[4,5-b]pyridin-2-yl)pyridin-2-amine

To a solution of Intermediate 31 (80 mg, 164 μmol) and 2,4-dichloro-1,3,5-triazine (27.1 mg, 180 μmol) in THF (4 mL) and DMF (4 mL) was added DIEA (106 mg, 820 μmol). The mixture was stirred at 0° C. for 2 hr. The reaction was filtered and concentrated under reduced pressure. The residue was purified by prep-HPLC (column: Waters xbridge 150*25 mm 10 μm; mobile phase: [water (NH₃H₂O)-ACN]; B %: 60%-90%, 10 min). 3-(3-(4-((8-(4-Chloro-1,3,5-triazin-2-yl)-3,8-diazabicyclo[3.2.1]octan-3-yl)methyl)phenyl)-5-phenyl-3H-imidazo[4,5-b]pyridin-2-yl)pyridin-2-amine (7.0 mg, yield: 7.1%) was obtained as a yellow solid. MS: m/z=601.3 [M+H]⁺. ¹H NMR (400 MHz, Dimethylsulfoxide-d₆) δ 8.46 (s, 1H), 8.27 (d, J=8.4 Hz, 1H), 8.03 (d, J=7.6 Hz, 2H), 7.98 (dd, J=5.6, 2.4 Hz, 2H), 7.51-7.36 (m, 7H), 7.12 (dd, J=8.0, 2.0 Hz, 1H), 7.05 (br s, 2H), 6.32 (dd, J=7.6, 4.8 Hz, 1H), 4.77-4.73 (m, 1H), 4.68-4.63 (m, 1H), 3.62 (s, 2H), 2.80-2.72 (m, 2H), 2.33-2.27 (m, 2H), 2.03-1.99 (m, 2H), 1.92-1.85 (m, 2H).

### Step 2: 4-(3-(4-(2-(2-Aminopyridin-3-yl)-5-phenyl-3H-imidazo[4,5-b]pyridin-3-yl)benzyl)-3,8-diazabicyclo[3.2.1]octan-8-yl)-1,3,5-triazine-2-carbonitrile

To a solution of 3-(3-(4-((8-(4-chloro-1,3,5-triazin-2-yl)-3,8-diazabicyclo[3.2.1]octan-3-yl)methyl)phenyl)-5-phenyl-3H-imidazo[4,5-b]pyridin-2-yl)pyridin-2-amine (30 mg, 49.9 μmol) in DMSO (1 mL) were added KCN (6.50 mg, 99.8 μmol) and DABCO (1.12 mg, 9.98 μmol). The mixture was stirred at 25° C. for 16 hr. The reaction was filtered and concentrated under reduced pressure. The mixture was quenched with H₂O (10 mL) and extracted with EtOAc (10 mL×3). The combined organic layers were dried over anhydrous Na₂SO₄, filtered and concentrated. The residue was purified by prep-TLC (CH₂Cl₂:MeOH=10:1) to give 4-(3-(4-(2-(2-aminopyridin-3-yl)-5-phenyl-3H-imidazo[4,5-b]pyridin-3-yl)benzyl)-3,8-diazabicyclo[3.2.1]octan-8-yl)-1,3,5-triazine-2-carbonitrile (Example 270, 9.4 mg, yield: 32%) as a yellow solid. MS: m/z=592.4 [M+H]⁺. ¹H NMR (400 MHz, Chloroform-d) δ 8.52 (s, 1H), 8.14 (d, J=8.4 Hz, 1H), 8.08 (dd, J=4.8, 1.6 Hz, 1H), 8.04-8.01 (m, 2H), 7.81 (d, J=8.4 Hz, 1H), 7.50-7.38 (m, 7H), 7.09 (dd, J=7.6, 1.6 Hz, 1H), 6.63 (br s, 2H), 6.36 (dd, J=7.6, 4.8 Hz, 1H), 4.83-4.79 (m, 2H), 3.62 (s, 2H), 2.86-2.80 (m, 2H), 2.42-2.36 (m, 2H), 2.13-2.10 (m, 2H), 1.99-1.95 (m, 2H).

### Example 271: 4-(8-(4-(2-(2-Aminopyridin-3-yl)-5-phenyl-3H-imidazo[4,5-b]pyridin-3-yl)benzyl)-3,8-diazabicyclo[3.2.1]octan-3-yl)-1,3,5-triazine-2-carbonitrile

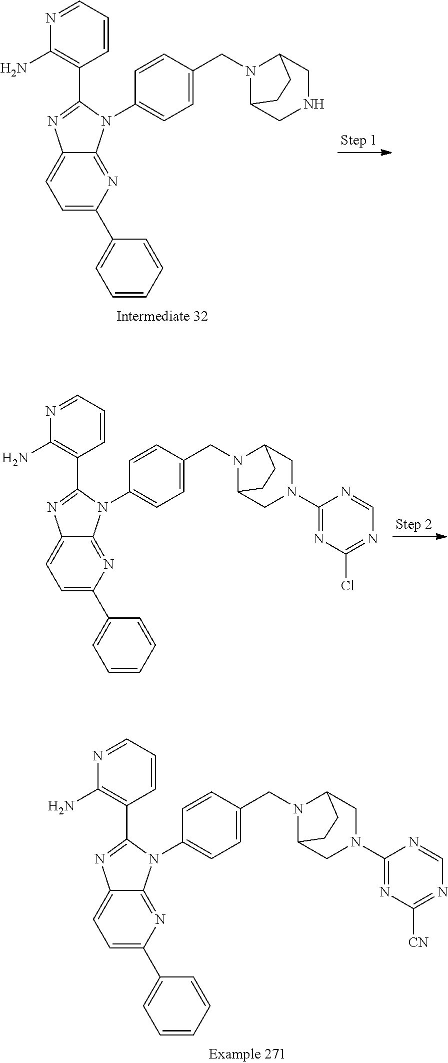 [SMILES: Nc1ncccc1-c1nc2ccc(-c3ccccc3)nc2n1-c1ccc(CN2C3CCC2CNC3)cc1 ; Nc1ncccc1-c1nc2ccc(-c3ccccc3)nc2n1-c1ccc(CN2C3CCC2CN(c2ncnc(Cl)n2)C3)cc1 ; N#Cc1ncnc(N2CC3CCC(C2)N3Cc2ccc(-n3c(-c4cccnc4N)nc4ccc(-c5ccccc5)nc43)cc2)n1]

### Step 1: 3-(3-(4-((3-(4-Chloro-1,3,5-triazin-2-yl)-3,8-diazabicyclo[3.2.1]octan-8-yl)methyl)phenyl)-5-phenyl-3H-imidazo[4,5-b]pyridin-2-yl)pyridin-2-amine

To a solution of Intermediate 32 (50 mg, 103 μmol) and 2,4-dichloro-1,3,5-triazine (18.4 mg, 123 μmol) in THF (2 mL) and DMF (2 mL) was added DIEA (66.3 mg, 513 μmol). The mixture was stirred at 25° C. for 2 hr, and then filtered and concentrated under reduced pressure. The residue was purified by prep-HPLC(column: Waters xbridge 150*25 mm 10 m; mobile phase: [water (NH₄HCO₃)-ACN]; B %: 56%-86%, 10 min) to 3-(3-(4-((3-(4-chloro-1,3,5-triazin-2-yl)-3,8-diazabicyclo[3.2.1]octan-8-yl)methyl)phenyl)-5-phenyl-3H-imidazo[4,5-b]pyridin-2-yl)pyridin-2-amine (40 mg, 65% yield) as a light-yellow solid. MS: m/z=601.3 [M+H]⁺. ¹H NMR (400 MHz, Dimethylsulfoxide-d₆) δ 8.47 (s, 1H), 8.27 (d, J=8.4 Hz, 1H), 8.05-7.95 (m, 4H), 7.60 (d, J=8.4 Hz, 2H), 7.50-7.39 (m, 5H), 7.17 (dd, J=7.6, 1.6 Hz, 1H), 7.00 (br s, 2H), 6.41 (dd, J=7.6, 4.8 Hz, 1H), 4.32-4.27 (m, 1H), 4.20-4.15 (m, 1H), 3.67 (s, 2H), 3.24-3.18 (m, 2H), 2.55-2.52 (m, 2H), 2.04-1.98 (m, 2H), 1.54-1.47 (m, 2H).

### Step 2: 4-(8-(4-(2-(2-Aminopyridin-3-yl)-5-phenyl-3H-imidazo[4,5-b]pyridin-3-yl)benzyl)-3,8-diazabicyclo[3.2.1]octan-3-yl)-1,3,5-triazine-2-carbonitrile

To a solution of 3-(3-(4-((3-(4-chloro-1,3,5-triazin-2-yl)-3,8-diazabicyclo[3.2.1]octan-8-yl)methyl)phenyl)-5-phenyl-3H-imidazo[4,5-b]pyridin-2-yl)pyridin-2-amine (30.0 mg, 49.9 μmol) in DMSO (1 mL) were added KCN (6.50 mg, 99.8 μmol) and DABCO (1.12 mg, 9.98 μmol). The mixture was stirred at 25° C. for 16 hr. The reaction was filtered and concentrated under reduced pressure. The mixture was quenched with H₂O (10 mL) and extracted with EtOAc (10 mL×3). The combined organic layers were dried over anhydrous Na₂SO₄, filtered and concentrated. The residue was purified by prep-TLC (CH₂Cl₂:MeOH=10:1) to give 4-(8-(4-(2-(2-aminopyridin-3-yl)-5-phenyl-3H-imidazo[4,5-b]pyridin-3-yl)benzyl)-3,8-diazabicyclo[3.2.1]octan-3-yl)-1,3,5-triazine-2-carbonitrile (Example 271, 4.3 mg, yield: 15%) as a yellow solid. MS: m/z=592.4 [M+H]⁺. ¹H NMR (400 MHz, Chloroform-d) δ 8.55 (s, 1H), 8.14 (d, J=8.4 Hz, 1H), 8.08 (d, J=3.6 Hz, 1H), 8.04-8.01 (m, 2H), 7.81 (d, J=8.4 Hz, 1H), 7.57 (d, J=8.0 Hz, 2H) 7.47-7.38 (m, 5H), 7.13 (dd, J=8.0, 1.2 Hz, 1H), 6.70 (br s, 2H), 6.39 (dd, J=7.6, 4.8 Hz, 1H), 4.42-4.33 (m, 2H), 3.69 (s, 2H), 3.40-3.36 (m, 2H), 3.27 (d, J=12.8 Hz, 2H), 2.12-2.06 (m, 2H), 1.32-1.30 (m, 2H).

### Example 272: 4-((1R,4R)-5-(4-(2-(2-aminopyridin-3-yl)-5-phenyl-3H-imidazo[4,5-b]pyridin-3-yl)benzyl)-2,5-diazabicyclo[2.2.1]heptan-2-yl)-1,3,5-tri azine-2-carbonitrile

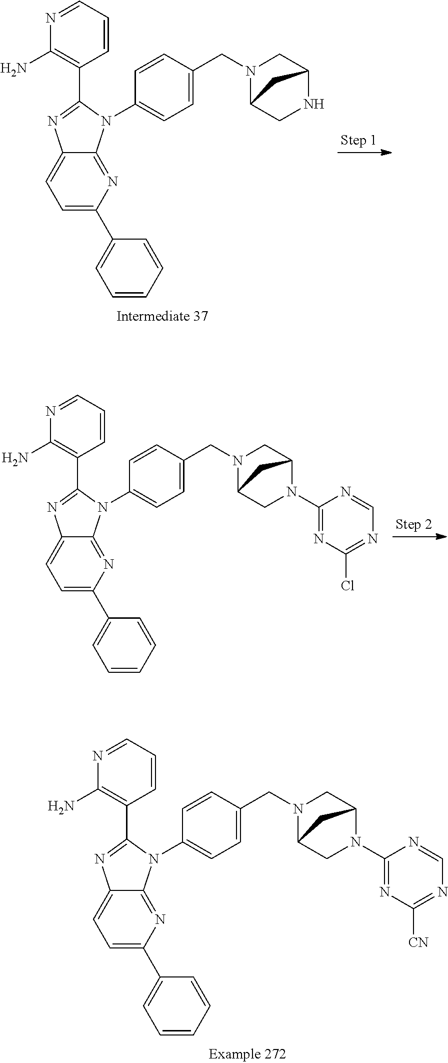 [SMILES: Nc1ncccc1-c1nc2ccc(-c3ccccc3)nc2n1-c1ccc(CN2C[C@H]3C[C@@H]2CN3)cc1 ; Nc1ncccc1-c1nc2ccc(-c3ccccc3)nc2n1-c1ccc(CN2C[C@H]3C[C@@H]2CN3c2ncnc(Cl)n2)cc1 ; N#Cc1ncnc(N2C[C@H]3C[C@@H]2CN3Cc2ccc(-n3c(-c4cccnc4N)nc4ccc(-c5ccccc5)nc43)cc2)n1]

### Step 1: 3-(3-(4-(((1R,4R)-5-(4-Chloro-1,3,5-triazin-2-yl)-2,5-diazabicyclo[2.2.1]heptan-2-yl)methyl)phenyl)-5-phenyl-3H-imidazo[4,5-b]pyridin-2-yl)pyridin-2-amine

To a solution of Intermediate 37 (80 mg, 168 μmol) and 2,4-dichloro-1,3,5-triazine (27.8 mg, 185 μmol) in THF (4 mL) and DMF (4 mL) was added DIEA (109 mg, 844 μmol). The mixture was stirred at 0° C. for 2 hr. The reaction mixture was quenched with H₂O (20 mL) and extracted with EtOAc (20 mL×3). The combined organic layers were washed with brine (20 mL×2), dried over Na₂SO₄, filtered and concentrated under reduced pressure. The residue was purified by prep-HPLC (column: Waters xbridge 150*25 mm 10 μm; mobile phase: [water (NH₃H₂O)-ACN]; B %: 46%-76%, 10 min) to give 3-(3-(4-(((1R,4R)-5-(4-chloro-1,3,5-triazin-2-yl)-2,5-diazabicyclo[2.2.1]heptan-2-yl)methyl)phenyl)-5-phenyl-3H-imidazo[4,5-b]pyridin-2-yl)pyridin-2-amine (21.7 mg, yield: 22%) as a yellow solid. MS: m/z=587.3 [M+H]⁺. ¹H NMR (400 MHz, Methanol-d₄) δ 8.33 (d, J=12.8 Hz, 1H), 8.18 (d, J=8.4 Hz, 1H), 8.05-8.02 (m, 2H), 7.98 (dd, J=4.8, 1.6 Hz, 1H), 7.93 (d, J=8.4 Hz, 1H), 7.56 (d, J=8.0 Hz, 2H), 7.45-7.30 (m, 6H), 6.49-6.45 (m, 1H), 4.98-4.92 (m, 1H), 3.89 (s, 2H), 3.85-3.79 (m, 1H), 3.75-3.72 (m, 1H), 3.49-3.44 (m, 1H), 3.06-2.98 (m, 1H), 2.76-2.27 (m, 1H), 2.11 (d, J=10.4 Hz, 1H), 1.89 (d, J=10.0 Hz, 1H).

### Step 2: 4-((1R,4R)-5-(4-(2-(2-Aminopyridin-3-yl)-5-phenyl-3H-imidazo[4,5-b]pyridin-3-yl)benzyl)-2,5-diazabicyclo[2.2.1]heptan-2-yl)-1,3,5-triazine-2-carbonitrile

To a solution of 3-(3-(4-(((1R,4R)-5-(4-chloro-1,3,5-triazin-2-yl)-2,5-diazabicyclo[2.2.1]heptan-2-yl)methyl)phenyl)-5-phenyl-3H-imidazo[4,5-b]pyridin-2-yl)pyridin-2-amine (60 mg, 102 μmol) in DMSO (1 mL) was added KCN (13.3 mg, 204 μmol) and DABCO (2.29 mg, 20.4 μmol). The mixture was stirred at 25° C. for 16 hr. The mixture was quenched with H₂O (10 mL) and extracted with EtOAc (10 mL×3), the combined organic layers were dried over anhydrous Na₂SO₄, filtered and concentrated. The residue was purified by prep-TLC (CH₂Cl₂:MeOH=10:1) to give 4-((1R,4R)-5-(4-(2-(2-aminopyridin-3-yl)-5-phenyl-3H-imidazo[4,5-b]pyridin-3-yl)benzyl)-2,5-diazabicyclo[2.2.1]heptan-2-yl)-1,3,5-triazine-2-carbonitrile (Example 272, 13.4 mg, yield: 23%) as a yellow solid. MS: m/z=578.3 [M+H]⁺. ¹H NMR (400 MHz, Chloroform-d) δ 8.54 (d, J=18.4 Hz, 1H), 8.13 (d, J=8.4 Hz, 1H), 8.08-8.05 (m, 1H), 8.01 (d, J=7.2 Hz, 2H), 7.80 (d, J=8.4 Hz, 1H), 7.52-7.35 (m, 7H), 7.09 (dd, J=8.0, 1.6 Hz, 1H), 6.62 (s, 2H), 6.38-6.34 (m, 1H), 4.98-4.95 (m, 1H), 3.87 (s, 2H), 3.85-3.81 (m, 1H), 3.75-3.70 (m, 1H), 3.47-3.42 (m, 1H), 3.06 (d, J=10.0 Hz, 1H), 2.72 (dd, J=13.2, 10.8 Hz, 1H), 2.12 (d, J=10.0 Hz, 1H), 1.87 (d, J=10.0 Hz, 1H).

### Example 273: (R)-4-(4-(4-(2-(2-Aminopyridin-3-yl)-5-phenyl-3H-imidazo[4,5-b]pyridin-3-yl)benzyl)-2-methylpiperazin-1-yl)-1,3,5-tri azine-2-carbonitrile

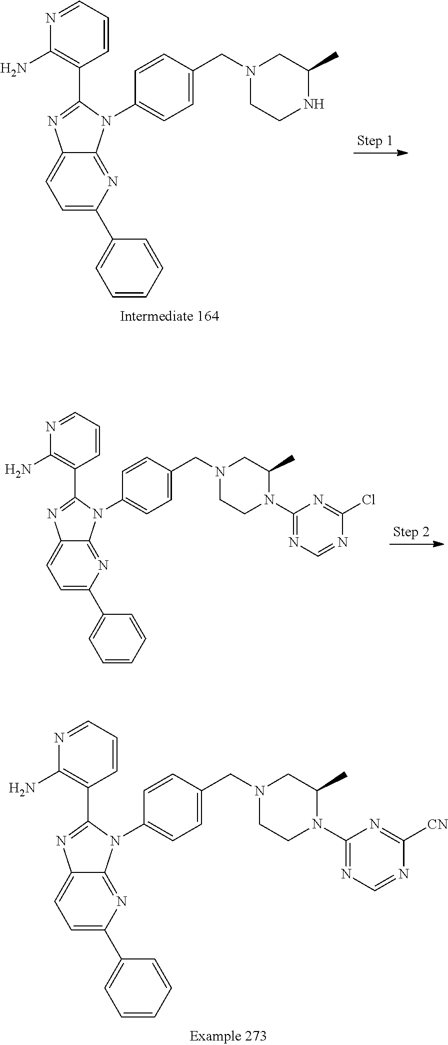 [SMILES: C[C@@H]1CN(Cc2ccc(-n3c(-c4cccnc4N)nc4ccc(-c5ccccc5)nc43)cc2)CCN1 ; C[C@@H]1CN(Cc2ccc(-n3c(-c4cccnc4N)nc4ccc(-c5ccccc5)nc43)cc2)CCN1c1ncnc(Cl)n1 ; C[C@@H]1CN(Cc2ccc(-n3c(-c4cccnc4N)nc4ccc(-c5ccccc5)nc43)cc2)CCN1c1ncnc(C#N)n1]

### Step 1: (R)-3-(3-(4-((4-(4-Chloro-1,3,5-triazin-2-yl)-3-methylpiperazin-1-yl)methyl)phenyl)-5-phenyl-3H-imidazo[4,5-b]pyridin-2-yl)pyridin-2-amine

To a solution of Intermediate 164 (100 mg, 210 μmol) in THF (10 mL) was added DIEA (81.5 mg, 631 μmol). Then 2,4-dichloro-1,3,5-triazine (35 mg, 231 μmol) in THF (3 mL) was added into the mixture at 0° C. and the reaction mixture was stirred at 0° C. for 1 hr. The mixture was diluted with H₂O (5 mL) and extracted with CH₂Cl₂ (15 mL×2). The combined organic layers were washed with brine (15 mL), dried over Na₂SO₄, filtered and concentrated under reduced pressure (≤35° C.) to give a crude product. After purified by silica gel flash chromatography (Eluent of 1%˜3% MeOH in CH₂Cl₂), (R)-3-(3-(4-((4-(4-chloro-1,3,5-triazin-2-yl)-3-methylpiperazin-1-yl)methyl)phenyl)-5-phenyl-3H-imidazo[4,5-b]pyridin-2-yl)pyridin-2-amine (40 mg, yield: 31%) was obtained as a yellow solid. MS: m/z=589.3 [M+H]⁺.

### Step 2. (R)-4-(4-(4-(2-(2-Aminopyridin-3-yl)-5-phenyl-3H-imidazo[4,5-b]pyridin-3-yl)benzyl)-2-methylpiperazin-1-yl)-1,3,5-triazine-2-carbonitrile

To a solution of (R)-3-(3-(4-((4-(4-chloro-1,3,5-triazin-2-yl)-3-methylpiperazin-1-yl)methyl)phenyl)-5-phenyl-3H-imidazo[4,5-b]pyridin-2-yl)pyridin-2-amine (40 mg, 67.9 μmol) in DMSO (1 mL) was added KCN (8.84 mg, 136 μmol) and DABCO (1.52 mg, 13.6 μmol). The mixture was stirred at 25° C. for 16 hr. The reaction mixture was poured into H₂O (10 mL). The resulting mixture was extracted with EtOAc (10 mL×2). The combined organic layers were washed with brine (10 mL), dried over Na₂SO₄, filtered and concentrated under reduced pressure. The crude product was purified by prep-TLC (100% EtOAc) to give (R)-4-(4-(4-(2-(2-aminopyridin-3-yl)-5-phenyl-3H-imidazo[4,5-b]pyridin-3-yl)benzyl)-2-methylpiperazin-1-yl)-1,3,5-triazine-2-carbonitrile (Example 273, 8 mg, yield: 18%) as a yellow solid. MS: m/z=580.3 [M+H]⁺. ¹H NMR (400 MHz, Acetonitrile-d₃) δ 8.56 (d, J=4.8 Hz, 1H), 8.18 (d, J=8.4 Hz, 1H), 8.05-8.02 (m, 3H), 7.90 (d, J=8.0 Hz, 1H), 7.57 (d, J=8.4 Hz, 2H), 7.49-7.39 (m, 5H), 7.16 (dd, J=8.0, 1.6 Hz, 1H), 6.60 (br s, 1H), 6.39 (dd, J=8.0, 4.8 Hz, 1H), 4.95-4.74 (m, 1H), 4.60-4.40 (m, 1H), 3.72-3.54 (m, 2H), 3.36-3.23 (m, 1H), 3.02-2.91 (m, 1H), 2.84-2.73 (m, 1H), 2.29-2.24 (m, 1H), 2.18-2.16 (m, 1H), 1.35-1.32 (m, 3H).

### Example 274: (S)-4-(4-(4-(2-(2-Aminopyridin-3-yl)-5-phenyl-3H-imidazo[4,5-b]pyridin-3-yl)benzyl)-2-methylpiperazin-1-yl)-1,3,5-triazine-2-carbonitrile

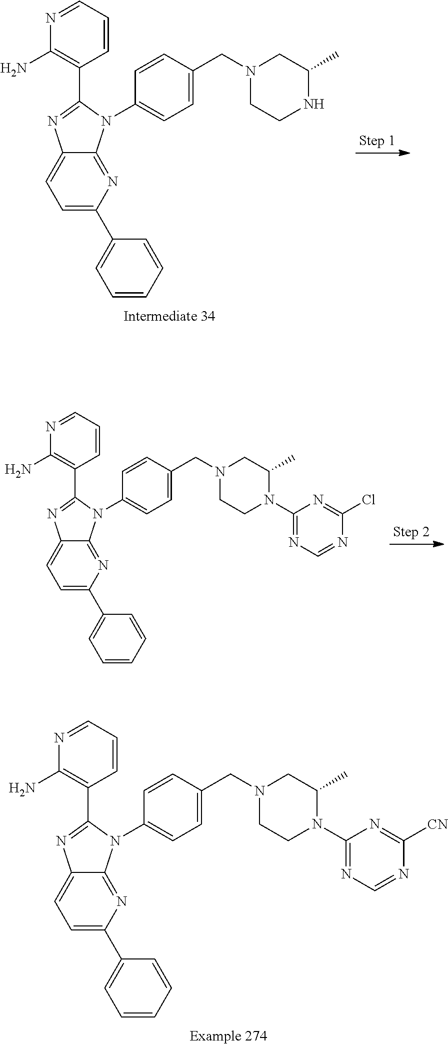 [SMILES: C[C@H]1CN(Cc2ccc(-n3c(-c4cccnc4N)nc4ccc(-c5ccccc5)nc43)cc2)CCN1 ; C[C@H]1CN(Cc2ccc(-n3c(-c4cccnc4N)nc4ccc(-c5ccccc5)nc43)cc2)CCN1c1ncnc(Cl)n1 ; C[C@H]1CN(Cc2ccc(-n3c(-c4cccnc4N)nc4ccc(-c5ccccc5)nc43)cc2)CCN1c1ncnc(C#N)n1]

### Step 1: (S)-3-(3-(4-((4-(4-Chloro-1,3,5-triazin-2-yl)-3-methylpiperazin-1-yl)methyl)phenyl)-5-phenyl-3H-imidazo[4,5-b]pyridin-2-yl)pyridin-2-amine

Following the general procedure of Example 273, the reaction of Intermediate 34 (100 mg, 210 μmol) with 2,4-dichloro-1,3,5-triazine (35 mg, 231 μmol) was carried out. After purified by silica gel flash chromatography (Eluent of 10˜65% EtOAc in petroleum ether), (S)-3-(3-(4-((4-(4-chloro-1,3,5-triazin-2-yl)-3-methylpiperazin-1-yl)methyl)phenyl)-5-phenyl-3H-imidazo[4,5-b]pyridin-2-yl)pyridin-2-amine (40 mg, yield: 25%) was obtained as a yellow solid. MS: m/z=589.4 [M+H]⁺.

### Step 2: (S)-4-(4-(4-(2-(2-Aminopyridin-3-yl)-5-phenyl-3H-imidazo[4,5-b]pyridin-3-yl)benzyl)-2-methylpiperazin-1-yl)-1,3,5-triazine-2-carbonitrile

To a solution of (S)-3-(3-(4-((4-(4-chloro-1,3,5-triazin-2-yl)-3-methylpiperazin-1-yl)methyl)phenyl)-5-phenyl-3H-imidazo[4,5-b]pyridin-2-yl)pyridin-2-amine (40 mg, 67.9 μmol) in DMSO (1 mL) were added KCN (8 mg, 136 μmol) and then DABCO (1.52 mg, 13.6 μmol). The mixture was stirred at 25° C. for 16 hr. The reaction mixture was poured into H₂O (10 mL). The resulting mixture was extracted with EtOAc (10 mL×2). The combined organic layers were washed with brine (10 mL), dried over Na₂SO₄, filtered and concentrated under reduced pressure. The crude product was purified by prep-TLC (100% EtOAc) to give (S)-4-(4-(4-(2-(2-aminopyridin-3-yl)-5-phenyl-3H-imidazo[4,5-b]pyridin-3-yl)benzyl)-2-methylpiperazin-1-yl)-1,3,5-triazine-2-carbonitrile (Example 274, 13.8 mg, yield: 33%) as a yellow solid. MS: m/z=580.4 [M+H]⁺. ¹H NMR (400 MHz, Acetonitrile-d₃) δ 8.57 (d, J=4.4 Hz, 1H), 8.19 (d, J=8.4 Hz, 1H), 8.07-8.01 (m, 3H), 7.91 (d, J=8.0 Hz, 1H), 7.58 (d, J=8.0 Hz, 2H), 7.50-7.40 (m, 5H), 7.19-7.14 (m, 1H), 6.62 (br s, 2H), 6.40 (dd, J=7.6, 5.2 Hz, 1H), 4.99-4.74 (m, 1H), 4.63-4.39 (m, 1H), 3.73-3.55 (m, 2H), 3.36-3.27 (m, 1H), 3.03-2.95 (m, 1H), 2.84-2.75 (m, 1H), 2.29-2.23 (m, 1H), 2.13-2.09 (m, 1H), 1.35-1.32 (m, 3H).

### Example 275: 4-((2S,6S)-4-(4-(2-(2-Aminopyridin-3-yl)-5-phenyl-3H-imidazo[4,5-b]pyridin-3-yl)benzyl)-2,6-dimethylpiperazin-1-yl)-1,3,5-triazine-2-carbonitrile

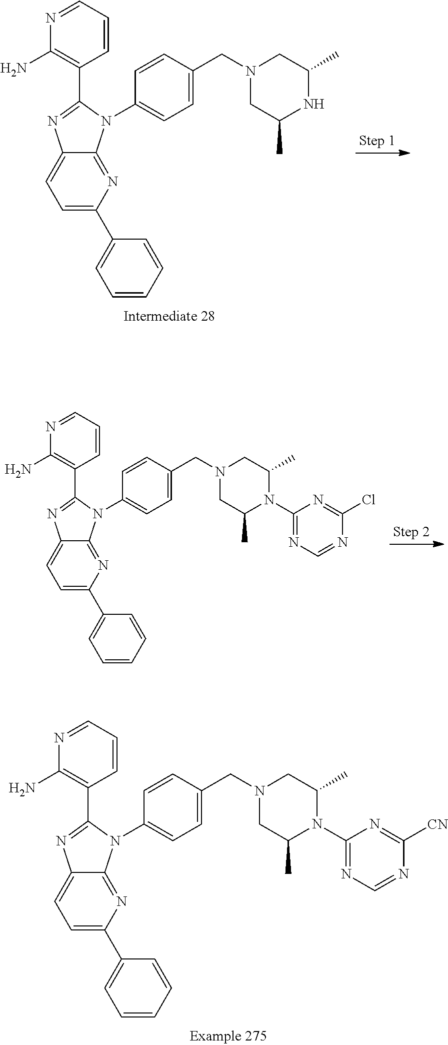 [SMILES: C[C@H]1CN(Cc2ccc(-n3c(-c4cccnc4N)nc4ccc(-c5ccccc5)nc43)cc2)C[C@H](C)N1 ; C[C@H]1CN(Cc2ccc(-n3c(-c4cccnc4N)nc4ccc(-c5ccccc5)nc43)cc2)C[C@H](C)N1c1ncnc(Cl)n1 ; C[C@H]1CN(Cc2ccc(-n3c(-c4cccnc4N)nc4ccc(-c5ccccc5)nc43)cc2)C[C@H](C)N1c1ncnc(C#N)n1]

### Step 1: 3-(3-(4-(((3S,5S)-4-(4-Chloro-1,3,5-triazin-2-yl)-3,5-dimethylpiperazin-1-yl)methyl)phenyl)-5-phenyl-3H-imidazo[4,5-b]pyridin-2-yl)pyridin-2-amine

To a solution of Intermediate 28 (100 mg, 204 μmol) in THF (5 mL) and DMF (5 M1) was added DIEA (79.2 mg, 613 μmol). Then 2,4-dichloro-1,3,5-triazine (35 mg, 231 μmol) in THF (3 mL) was added into the mixture at 0° C. and the reaction mixture was stirred at 0° C. for 1 hr. The mixture was diluted with H₂O (5 mL) and extracted with CH₂Cl₂ (15 mL×2). The combined organic layers were washed with brine (15 mL), dried over Na₂SO₄, filtered and concentrated under reduced pressure (≤35° C.) to give a crude product. After purified by prep-TLC (CH₂Cl₂:MeOH=10:1), 3-(3-(4-(((3S,5S)-4-(4-chloro-1,3,5-triazin-2-yl)-3,5-dimethylpiperazin-1-yl)methyl)phenyl)-5-phenyl-3H-imidazo[4,5-b]pyridin-2-yl)pyridin-2-amine (50 mg, yield: 33%) was obtained as a yellow solid. MS: m/z=603.4 [M+H]⁺. ¹H NMR (400 MHz, Methanol-d₄) δ 8.39 (s, 1H), 8.20 (d, J=8.4 Hz, 1H), 8.06-8.03 (m, 2H), 7.99 (dd, J=5.2, 2.0 Hz, 1H), 7.95 (d, J=8.4 Hz, 1H), 7.58 (d, J=8.4 Hz, 2H), 7.47-7.37 (m, 5H), 7.34 (dd, J=7.6, 1.6 Hz, 1H), 6.49 (dd, 1=7.6, 4.8 Hz, 1H), 4.55-4.36 (m, 2H), 3.95 (d, J=13.2 Hz, 1H), 3.82 (d, J=13.2 Hz, 1H), 3.17-3.13 (m, 2H), 2.74 (dd, J=12.0, 4.0 Hz, 2H), 1.44 (d, J=6.4 Hz, 6H).

### Step 2: 4-((2S,6S)-4-(4-(2-(2-Aminopyridin-3-yl)-5-phenyl-3H-imidazo[4,5-b]pyridin-3-yl)benzyl)-2,6-dimethylpiperazin-1-yl)-1,3,5-triazine-2-carbonitrile

To a solution of 3-(3-(4-(((3S,5S)-4-(4-chloro-1,3,5-triazin-2-yl)-3,5-dimethylpiperazin-1-yl)methyl)phenyl)-5-phenyl-3H-imidazo[4,5-b]pyridin-2-yl)pyridin-2-amine (40 mg, 66.3 μmol) in DMSO (1 mL) were added KCN (8.64 mg, 133 μmol) and DABCO (1.49 mg, 13.3 μmol). The mixture was stirred at 25° C. for 16 hr. The reaction mixture was poured into H₂O (10 mL). The resulting mixture was extracted with EtOAc (10 mL×2). The combined organic layers were washed with brine (10 mL), dried over Na₂SO₄, filtered and concentrated under reduced pressure. The crude was purified by prep-TLC (100% EtOAc) to give 4-((2S,6S)-4-(4-(2-(2-aminopyridin-3-yl)-5-phenyl-3H-imidazo[4,5-b]pyridin-3-yl)benzyl)-2,6-dimethylpiperazin-1-yl)-1,3,5-triazine-2-carbonitrile (Example 275, 9.8 mg, yield: 22%) as a yellow solid. MS: m/z=594.4 [M+H]⁺. ¹H NMR (400 MHz, Acetonitrile-d₃) δ 8.59 (s, 1H), 8.17 (d, J=8.4 Hz, 1H), 8.05-7.96 (m, 3H), 7.90 (d, J=8.4 Hz, 1H), 7.60-7.53 (m, 2H), 7.49-7.39 (m, 5H), 7.19-7.11 (m, 1H), 6.62 (br s, 2H), 6.38 (dd, J=7.6, 4.8 Hz, 1H), 4.41 (br s, 2H), 3.96-3.78 (m, 2H), 3.22-3.11 (m, 2H), 2.74 (dd, J=12.4, 3.6 Hz, 2H), 1.40 (d, J=6.4 Hz, 6H).

### Example 276: 4-(7-(4-(2-(2-aminopyridin-3-yl)-5-phenyl-3H-imidazo[4,5-b]pyridin-3-yl)benzyl)-4,7-diazaspiro[2.5]octan-4-yl)-1,3,5-triazine-2-carbonitrile

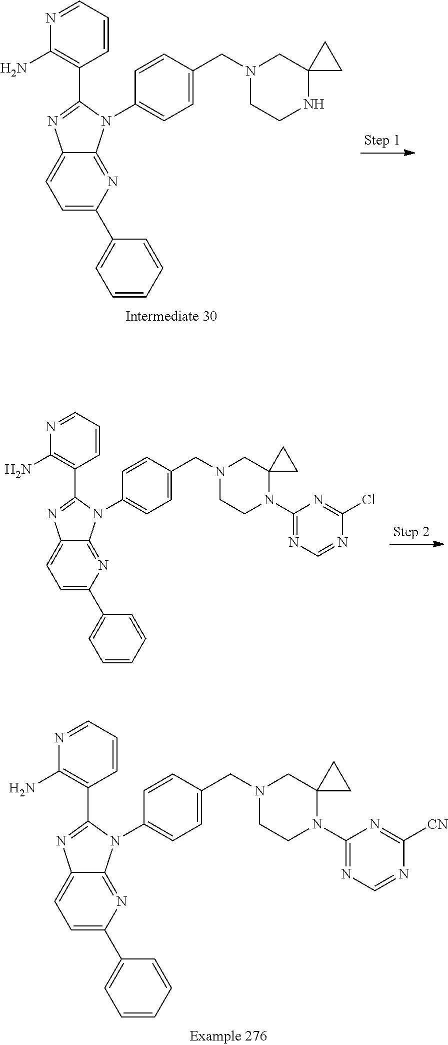 [SMILES: Nc1ncccc1-c1nc2ccc(-c3ccccc3)nc2n1-c1ccc(CN2CCNC3(CC3)C2)cc1 ; Nc1ncccc1-c1nc2ccc(-c3ccccc3)nc2n1-c1ccc(CN2CCN(c3ncnc(Cl)n3)C3(CC3)C2)cc1 ; N#Cc1ncnc(N2CCN(Cc3ccc(-n4c(-c5cccnc5N)nc5ccc(-c6ccccc6)nc54)cc3)CC23CC3)n1]

### Step 1: 3-(3-(4-((4-(4-Chloro-1,3,5-triazin-2-yl)-4,7-diazaspiro[2.5]octan-7-yl)methyl)phenyl)-5-phenyl-3H-imidazo[4,5-b]pyridin-2-yl)pyridin-2-amine

Following the general procedure of Example 273, the reaction of Intermediate 30 (150 mg, 308 μmol) with 2,4-dichloro-1,3,5-triazine (50.8 mg, 338 μmol) was carried out. After purified by silica gel flash chromatography (Eluent of 1˜100% EtOAc in petroleum ether), 3-(3-(4-((4-(4-chloro-1,3,5-triazin-2-yl)-4,7-diazaspiro[2.5]octan-7-yl)methyl)phenyl)-5-phenyl-3H-imidazo[4,5-b]pyridin-2-yl)pyridin-2-amine (45 mg, yield: 21%) was obtained as a yellow solid. MS: m/z=601.1 [M+H]⁺.

### Step 2: 4-(7-(4-(2-(2-Aminopyridin-3-yl)-5-phenyl-3H-imidazo[4,5-b]pyridin-3-yl)benzyl)-4,7-diazaspiro[2.5]octan-4-yl)-1,3,5-triazine-2-carbonitrile

To a solution of 3-(3-(4-((4-(4-chloro-1,3,5-triazin-2-yl)-4,7-diazaspiro[2.5]octan-7-yl)methyl)phenyl)-5-phenyl-3H-imidazo[4,5-b]pyridin-2-yl)pyridin-2-amine (45 mg, 74.9 μmol) in DMSO (1 mL) were added KCN (100 mg, 1.54 mmol) and then DABCO (1.68 mg, 15 μmol). The mixture was stirred at 25° C. for 16 hr. The reaction mixture was poured into H₂O (10 mL). The resulting mixture was extracted with EtOAc (10 mL×2). The combined organic layers were washed with brine (10 mL), dried over Na₂SO₄, filtered and concentrated under reduced pressure. The crude product was purified by prep-TLC (100% EtOAc) to give 4-(7-(4-(2-(2-aminopyridin-3-yl)-5-phenyl-3H-imidazo[4,5-b]pyridin-3-yl)benzyl)-4,7-diazaspiro[2.5]octan-4-yl)-1,3,5-triazine-2-carbonitrile (Example 276, 6.2 mg, yield: 14%) as an off-white solid. MS: m/z=592.3 [M+H]^{+ 1}H NMR (400 MHz, Acetonitrile-d₃) δ 8.60 (d, J=4.0 Hz, 1H), 8.18 (d, J=8.4 Hz, 1H), 8.05-8.00 (m, 3H), 7.91 (d, J=8.4 Hz, 1H), 7.54-7.41 (m, 7H), 7.14 (dd, J=7.6, 1.6 Hz, 1H), 6.58 (br s, 2H), 6.35 (dd, J=7.6, 4.8 Hz, 1H), 4.11-3.98 (m, 2H), 3.61 (s, 2H), 2.66-2.57 (m, 2H), 2.47-2.41 (m, 2H), 1.00-0.95 (m, 2H), 0.90-0.85 (m, 2H).

### Example 277: 4-((2R,6S)-4-(4-(2-(2-aminopyridin-3-yl)-5-phenyl-3H-imidazo[4,5-b]pyridin-3-yl)benzyl)-2,6-dimethylpiperazin-1-yl)-1,3,5-triazine-2-carbonitrile

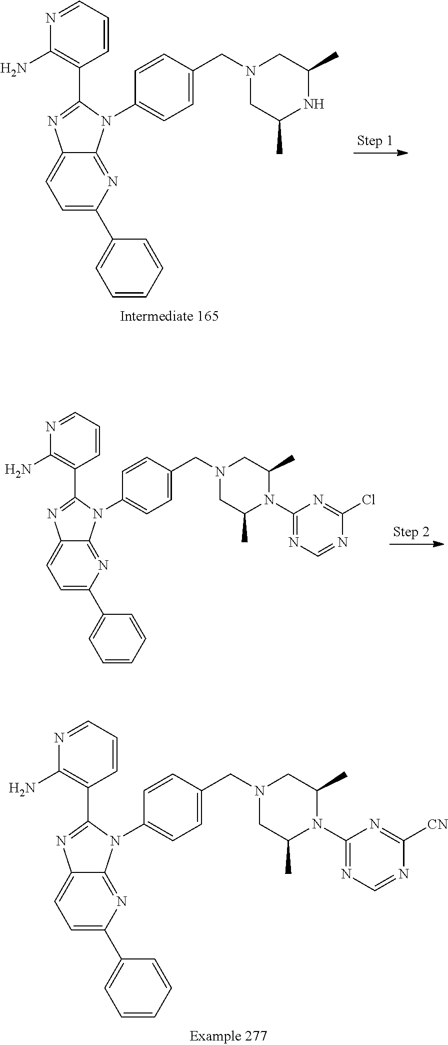 [SMILES: C[C@@H]1CN(Cc2ccc(-n3c(-c4cccnc4N)nc4ccc(-c5ccccc5)nc43)cc2)C[C@H](C)N1 ; C[C@@H]1CN(Cc2ccc(-n3c(-c4cccnc4N)nc4ccc(-c5ccccc5)nc43)cc2)C[C@H](C)N1c1ncnc(Cl)n1 ; C[C@@H]1CN(Cc2ccc(-n3c(-c4cccnc4N)nc4ccc(-c5ccccc5)nc43)cc2)C[C@H](C)N1c1ncnc(C#N)n1]

### Step 1: 3-(3-(4-(((3S,5R)-4-(4-Chloro-1,3,5-triazin-2-yl)-3,5-dimethylpiperazin-1-yl)methyl)phenyl)-5-phenyl-3H-imidazo[4,5-b]pyridin-2-yl)pyridin-2-amine

To a solution of Intermediate 165 (50 mg, 102 μmol) and K₂CO₃ (42.3 mg, 306 μmol) in THF (4 mL) and H₂O (1 mL) was added 2,4-dichloro-1,3,5-triazine (15.3 mg, 102 μmol). The mixture was stirred at 0° C. for 2 hr. The mixture was filtered and concentrated. The residue was purified by prep-HPLC (column: Waters xbridge 150×25 mm 10 μm; mobile phase: [water (NH₄HCO₃)-ACN]; B %: 61%-91%, 14 min) to give 3-(3-(4-(((3S,5R)-4-(4-chloro-1,3,5-triazin-2-yl)-3,5-dimethylpiperazin-1-yl)methyl)phenyl)-5-phenyl-3H-imidazo[4,5-b]pyridin-2-yl)pyridin-2-amine (1.8 mg, yield: 2.8%) as an off-white solid. MS: m/z=603.4 [M+H]⁺. ¹H NMR (400 MHz, Methanol-d₄) δ 8.38 (s, 1H), 8.20 (d, J=8.4 Hz, 1H), 8.08-8.03 (m, 2H), 7.99 (dd, J=5.2, 1.6 Hz, 1H), 7.97-7.92 (m, 1H), 7.63-7.61 (m, 2H), 7.48-7.44 (m, 2H), 7.43-7.39 (m, 2H), 7.38-7.32 (m, 2H), 6.49 (dd, 1=7.6, 4.8 Hz, 1H), 3.67 (s, 2H), 2.87-2.84 (m, 2H), 2.36-2.28 (M, 2H), 2.20-2.04 (m, 2H), 1.40 (d, J=7.2 Hz, 6H).

### Step 2: 4-((2R,6S)-4-(4-(2-(2-aminopyridin-3-yl)-5-phenyl-3H-imidazo[4,5-b]pyridin-3-yl)benzyl)-2,6-dimethylpiperazin-1-yl)-1,3,5-triazine-2-carbonitrile

To a solution of 3-(3-(4-(((3S,5R)-4-(4-chloro-1,3,5-triazin-2-yl)-3,5-dimethylpiperazin-1-yl)methyl)phenyl)-5-phenyl-3H-imidazo[4,5-b]pyridin-2-yl)pyridin-2-amine (55 mg, 91.2 μmol) in DMSO (1 mL) was added KCN (6 mg, 92.1 μmol). The mixture was stirred at 0° C. for 0.5 hr, then DABCO (2.05 mg, 18.2 μmol) was added. The mixture was stirred at 25° C. for 16 hr. The mixture was quenched with water (10 mL) and extracted with EtOAc (10 mL×3). The combined organic layers were dried over anhydrous Na₂SO₄, filtered and concentrated. The residue was purified by prep-HPLC (column: Waters xbridge 150×25 mm×10 μm; mobile phase: [water (NH₄HCO₃)-ACN]; B %: 58%-88%, 14 min) to give 4-((2R,6S)-4-(4-(2-(2-aminopyridin-3-yl)-5-phenyl-3H-imidazo[4,5-b]pyridin-3-yl)benzyl)-2,6-dimethylpiperazin-1-yl)-1,3,5-triazine-2-carbonitrile (Example 277, 5.9 mg, yield: 11%) as an off-white solid. MS: m/z=594.4 [M+H]⁺. ¹H NMR (400 MHz, Chloroform-d) δ 8.60 (s, 1H), 8.16 (d, J=8.4 Hz, 1H), 8.08-8.02 (m, 3H), 7.84 (d, J=8.4 Hz, 1H), 7.59 (d, J=8.4 Hz, 2H), 7.47-7.39 (m, 5H), 7.18-7.16 (m, 1H), 6.88 (br s, 2H), 6.39 (dd, J=7.6, 4.8 Hz, 1H), 4.78-4.70 (m, 2H), 3.67 (s, 2H), 2.84 (d, J=11.6 Hz, 2H), 2.38-2.32 (m, 2H), 1.41 (d, J=6.8 Hz, 6H).

### Example 278: 4-((1-(4-(2-(2-Aminopyridin-3-yl)-5-phenyl-3H-imidazo[4,5-b]pyridin-3-yl)benzyl)azepan-4-yl)amino)-1,3,5-triazine-2-carbonitrile

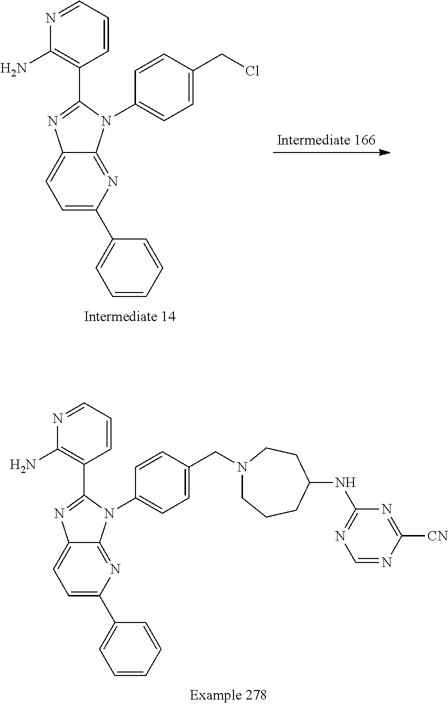 [SMILES: Nc1ncccc1-c1nc2ccc(-c3ccccc3)nc2n1-c1ccc(CCl)cc1 ; N#Cc1ncnc(NC2CCCN(Cc3ccc(-n4c(-c5cccnc5N)nc5ccc(-c6ccccc6)nc54)cc3)CC2)n1]

To a solution of Intermediate 14 (250 mg, 1.15 mmol) and Intermediate 166 (472 mg, 1.15 mmol) in DMF (3 mL) were added NaI (34.3 mg, 229.1 μmol) and K2CO₃ (475 mg, 3.44 mmol). The mixture was stirred at 25° C. for 16 hr. The reaction mixture was filtered to give a filter liquid. The residue was purified by prep-HPLC (column: Waters xbridge 150×25 mm×10 μm; mobile phase: [water (NH₄HCO₃)-ACN]; B %: 50%-80%, over 10 min) to give 4-((1-(4-(2-(2-aminopyridin-3-yl)-5-phenyl-3H-imidazo[4,5-b]pyridin-3-yl)benzyl)azepan-4-yl)amino)-1,3,5-triazine-2-carbonitrile (Example 278, 73 mg, yield: 9.9%) as yellow lyophilized powder. MS: m/z=594.4 [M+H]⁺. ¹H NMR (400 MHz, Dimethylsulfoxide-d₆) δ 8.92-8.89 (m, 1H), 8.71-8.54 (m, 1H), 8.27 (d, J=8.4 Hz, 1H), 8.04-7.97 (m, 4H), 7.54-7.49 (m, 2H), 7.48-7.43 (m, 4H), 7.40-7.36 (m, 1H), 7.15 (d, J=7.6 Hz, 1H), 7.05 (br s, 2H), 6.38-6.34 (m, 1H), 4.14-3.99 (m, 1H), 3.76-3.65 (m, 2H), 2.67-2.63 (m, 2H), 1.98-1.50 (m, 8H).

### Example 279: 4-((4-(4-(2-(2-Aminopyridin-3-yl)-5-phenyl-3H-imidazo[4,5-b]pyridin-3-yl)benzyl)-1,4-oxazepan-6-yl)amino)-1,3,5-triazine-2-carbonitrile

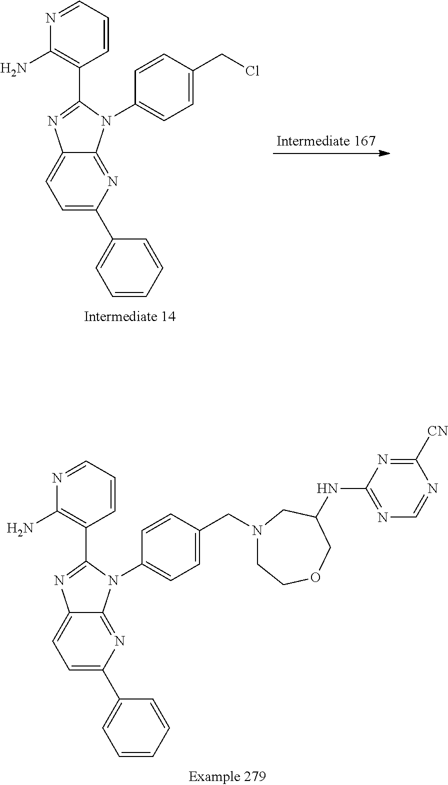 [SMILES: Nc1ncccc1-c1nc2ccc(-c3ccccc3)nc2n1-c1ccc(CCl)cc1 ; N#Cc1ncnc(NC2COCCN(Cc3ccc(-n4c(-c5cccnc5N)nc5ccc(-c6ccccc6)nc54)cc3)C2)n1]

To a solution of Intermediate 14 (330 mg, 736 μmol) and Intermediate 167 (246 mg, 736 μmol, TFA salt) in DMF (3 mL) were added K₂CO₃ (509 mg, 3.68 mmol) and NaI (55.2 mg, 368 μmol). The mixture was stirred at 50° C. for 2 hr. The mixture was quenched with H₂O (25 mL) and extracted with EtOAc (50 mL). The organic layer was dried over anhydrous Na₂SO₄, filtered and concentrated. The residue was purified by prep-TLC (CH₂Cl₂:MeOH=20:1) to give 4-((4-(4-(2-(2-aminopyridin-3-yl)-5-phenyl-3H-imidazo[4,5-b]pyridin-3-yl)benzyl)-1,4-oxazepan-6-yl)amino)-1,3,5-triazine-2-carbonitrile (Example 279, 55.4 mg, yield: 13%) as a light-yellow solid. MS: m/z=596.4 [M+H]⁺. ¹H NMR (400 MHz, Dimethylsulfoxide-d₆) δ 8.91-8.84 (m, 1H), 8.65-8.58 (m, 1H), 8.29-8.24 (m, 1H), 8.04-7.96 (m, 4H), 7.59-7.54 (m, 2H), 7.48-7.40 (m, 5H), 7.12-7.03 (m, 3H), 6.34-6.24 (m, 1H), 4.39-4.14 (m, 2H), 3.96-3.91 (m, 1H), 3.85-3.79 (m, 2H), 3.71-3.68 (m, 2H), 3.00-2.88 (m, 2H), 2.82-2.74 (m, 2H).

### Example 280: 4-((1-(4-(2-(2-Aminopyridin-3-yl)-5-methyl-3H-imidazo[4,5-b]pyridin-3-yl)benzyl)piperidin-4-yl)amino)pyrimidine-2-carbonitrile

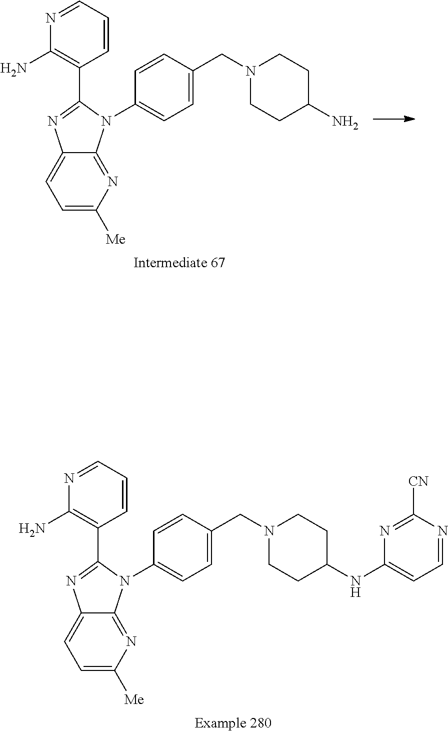 [SMILES: Cc1ccc2nc(-c3cccnc3N)n(-c3ccc(CN4CCC(N)CC4)cc3)c2n1 ; Cc1ccc2nc(-c3cccnc3N)n(-c3ccc(CN4CCC(Nc5ccnc(C#N)n5)CC4)cc3)c2n1]

A mixture of Intermediate 67 (80 mg, 193 μmol), 4-chloropyrimidine-2-carbonitrile (29.7 mg, 213 μmol) and DIEA (75 mg, 580 μmol) in NMP (2 mL) was taken up into a microwave tube. The sealed tube was stirred at 130° C. for 0.5 hr under microwave. The reaction mixture was poured into H₂O (10 mL), extracted with CH₂Cl₂ (15 mL). The organic phase was dried over anhydrous Na₂SO₄, filtered and concentrated under reduced pressure. The residue was purified by prep-HPLC (column: Phenomenex C18 150*25 mm*10 μm; mobile phase: [water (NH₄HCO₃)-ACN]; gradient: 22%-52% B over 14 min). 4-((1-(4-(2-(2-Aminopyridin-3-yl)-5-methyl-3H-imidazo[4,5-b]pyridin-3-yl)benzyl)piperidin-4-yl)amino)pyrimidine-2-carbonitrile (Example 280, 10.7 mg, yield: 11%) was obtained as an off-white lyophilized powder. MS: m/z=517.2 [M+H]⁺. ¹H NMR (400 MHz, Dimethylsulfoxide-d₆) δ 8.13-8.03 (m, 3H), 7.99 (dd, J=4.8, 2.0 Hz, 1H) 7.45 (d, J=8.0 Hz, 2H), 7.36 (d, J=8.4 Hz, 2H), 7.25 (d, J=8.0 Hz, 1H), 7.09 (dd, J=7.6, 1.6 Hz, 1H), 7.01 (br s, 2H), 6.68 (d, J=6.0 Hz, 1H), 6.34 (dd, J=8.0, 5.2 Hz, 1H), 3.89-3.74 (m, 1H), 3.57 (s, 2H), 2.85-2.79 (m, 2H), 2.52 (s, 3H), 2.19-2.10 (m, 2H), 1.94-1.85 (m, 2H), 1.57-1.43 (m, 2H).

### Example 281: 4-(4-(4-(2-(2-Aminopyridin-3-yl)-5-(2-fluorophenyl)-3H-imidazo[4,5-b]pyridin-3-yl)benzyl)piperazin-1-yl)pyrimidine-2-carbonitrile

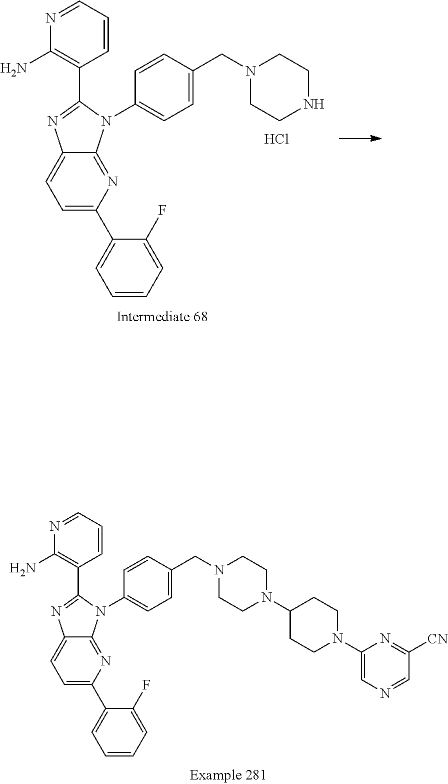 [SMILES: Nc1ncccc1-c1nc2ccc(-c3ccccc3F)nc2n1-c1ccc(CN2CCNCC2)cc1 ; Cl ; N#Cc1cncc(N2CCC(N3CCN(Cc4ccc(-n5c(-c6cccnc6N)nc6ccc(-c7ccccc7F)nc65)cc4)CC3)CC2)n1]

A mixture of Intermediate 68 (100 mg, 209 μmol), 4-chloropyrimidine-2-carbonitrile (29.1 mg, 209 μmol) and DIEA (27 mg, 209 μmol) in NMP (2 mL) was taken up into a microwave tube. The sealed tube was heated at 130° C. for 1 hr under microwave. After purified by prep-HPLC (column: Waters xbridge 150×25 mm×10 μm; mobile phase: [water (NH₄HCO₃)-ACN]; B %: 45%-75%, 10 min), 4-(4-(4-(2-(2-Aminopyridin-3-yl)-5-(2-fluorophenyl)-3H-imidazo[4,5-b]pyridin-3-yl)benzyl)piperazin-1-yl)pyrimidine-2-carbonitrile (Example 281, 18.1 mg, yield: 15%) was obtained as a yellow solid. MS: m/z=583.3 [M+H]⁺. ¹H NMR (400 MHz, Dimethylsulfoxide-d₆) δ 8.31-8.26 (m, 2H), 8.00 (dd, J=4.8, 1.2 Hz, 1H), 7.85-7.76 (m, 2H), 7.52-7.42 (m, 5H), 7.37-7.27 (m, 2H), 7.18-7.14 (m, 1H), 7.11 (d, J=6.4 Hz, 1H), 7.00 (br s, 2H), 6.37 (dd, J=7.2, 4.8 Hz, 1H), 3.80-3.63 (m, 4H), 3.62 (s, 2H), 2.49-2.46 (m, 4H). ¹⁹F NMR (400 MHz, Dimethylsulfoxide-d₆) δ −117.17.

### Example 282: 2-(4-(4-(2-(2-Aminopyridin-3-yl)-5-(2-fluorophenyl)-3H-imidazo[4,5-b]pyridin-3-yl)benzyl)piperazin-1-yl)pyrimidine-4-carbonitrile

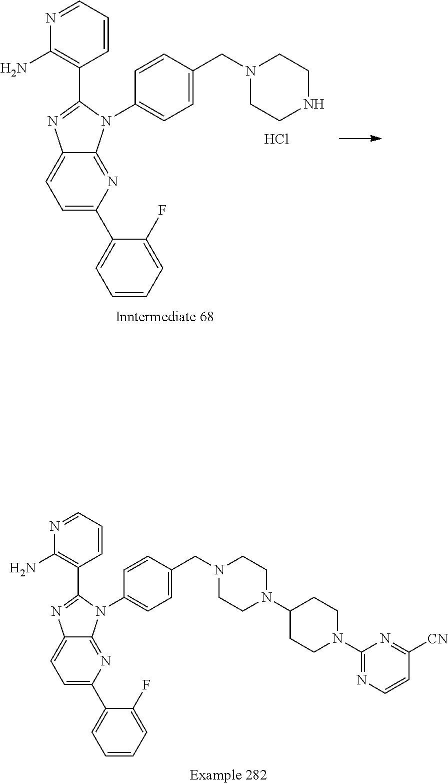 [SMILES: Nc1ncccc1-c1nc2ccc(-c3ccccc3F)nc2n1-c1ccc(CN2CCNCC2)cc1 ; Cl ; N#Cc1ccnc(N2CCC(N3CCN(Cc4ccc(-n5c(-c6cccnc6N)nc6ccc(-c7ccccc7F)nc65)cc4)CC3)CC2)n1]

A mixture of Intermediate 68 (80 mg, 167 μmol), 2-chloropyrimidine-4-carbonitrile (23.3 mg, 167 μmol) and DIEA (43.1 mg, 334 μmol) in NMP (2 mL) was taken up into a microwave tube. The sealed tube was heated at 130° C. for 1 hr under microwave. After purified by prep-HPLC (column: Waters xbridge 150×25 mm×10 μm; mobile phase: [water (NH₄HCO₃)-ACN]; B %: 65%-95%, 10 min), 2-(4-(4-(2-(2-Aminopyridin-3-yl)-5-(2-fluorophenyl)-3H-imidazo[4,5-b]pyridin-3-yl)benzyl)piperazin-1-yl)pyrimidine-4-carbonitrile (Example 282, 60.4 mg, yield: 62%) was obtained as a brown solid. MS: m/z=583.3 [M+H]⁺. ¹H NMR (400 MHz, Dimethylsulfoxide-d₆) δ 8.64 (d, J=4.8 Hz, 1H), 8.30 (d, J=8.4 Hz, 1H), 8.00 (dd, J=4.8, 2.0 Hz, 1H), 7.84-7.76 (m, 2H), 7.51-7.43 (m, 5H), 7.38-7.30 (m, 2H), 7.20-7.13 (m, 2H), 6.99 (br s, 2H), 6.38 (dd, J=7.6, 4.8 Hz, 1H), 3.80-3.73 (m, 4H), 3.62 (s, 2H), 2.54-2.52 (m, 4H). ¹⁹F NMR (400 MHz, Dimethylsulfoxide-dₕ) 5-117.18.

### Example 283: 2-(4-(4-(2-(2-Aminopyridin-3-yl)-5-(3-oxo-1,4-diazepan-1-yl)-3H-imidazo[4,5-b]pyridin-3-yl)benzyl)piperazin-1-yl)pyrimidine-4-carbonitrile

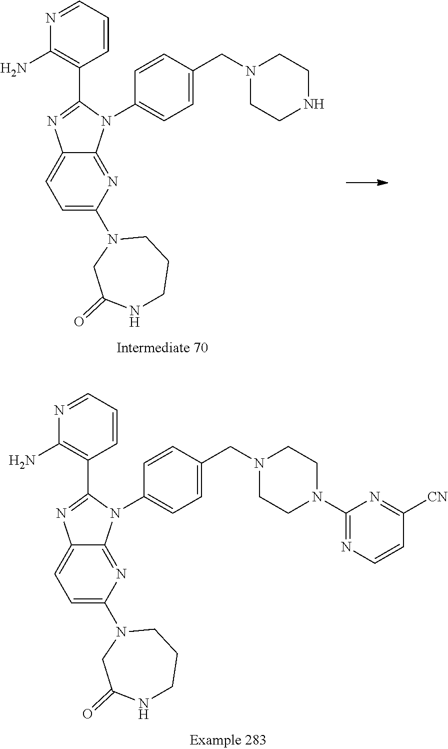 [SMILES: Nc1ncccc1-c1nc2ccc(N3CCCNC(=O)C3)nc2n1-c1ccc(CN2CCNCC2)cc1 ; N#Cc1ccnc(N2CCN(Cc3ccc(-n4c(-c5cccnc5N)nc5ccc(N6CCCNC(=O)C6)nc54)cc3)CC2)n1]

A mixture of Intermediate 70 (250 mg, 502 μmol), 2-chloropyrimidine-4-carbonitrile (70.1 mg, 502 μmol) and DIEA (260 mg, 2.01 mmol) in NMP (3 mL) was taken up into a microwave tube. The sealed tube was heated at 130° C. for 1 hr under microwave. The reaction mixture was quenched with H₂O (5 mL) at 0° C., and then extracted with CH₂Cl₂ (15 mL×3). The combined organic layers were washed with brine (15 mL), dried over Na₂SO₄, filtered and concentrated under reduced pressure. After purified by prep-HPLC (column: Phenomenex C18 150*25 mm*10 μm; mobile phase: [water (NH₄HCO₃)-ACN]; B %: 30%-60%, over 10 min), 2-(4-(4-(2-(2-aminopyridin-3-yl)-5-(3-oxo-1,4-diazepan-1-yl)-3H-imidazo[4,5-b]pyridin-3-yl)benzyl)piperazin-1-yl)pyrimidine-4-carbonitrile (Example 283, 98.8 mg, yield: 33%) was obtained as a yellow solid. MS: m/z=601.4 [M+H]⁺. ¹H NMR (400 MHz, Dimethylsulfoxide-d₆) δ 8.64 (d, J=4.8 Hz, 1H), 8.00-7.89 (m, 2H), 7.47-7.42 (m, 3H), 7.34 (d, J=8.0 Hz, 2H), 7.15 (d, J=4.4 Hz, 1H), 7.03-6.95 (m, 3H), 6.80 (d, J=8.8 Hz, 1H), 6.32 (dd, J=7.6, 4.8 Hz, 1H), 4.18 (s, 2H), 3.86-3.73 (m, 6H), 3.61 (s, 2H), 3.20-3.15 (m, 2H), 2.48-2.46 (m, 4H), 1.68-1.61 (m, 2H).

### Example 284: 6-(4-(4-(2-(2-Aminopyridin-3-yl)-5-(3-oxo-1,4-diazepan-1-yl)-3H-imidazo[4,5-b]pyridin-3-yl)benzyl)piperazin-1-yl)pyrimidine-4-carbonitrile

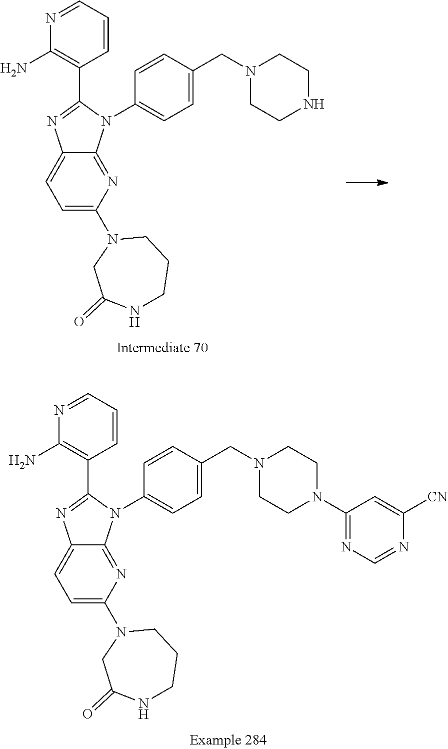 [SMILES: Nc1ncccc1-c1nc2ccc(N3CCCNC(=O)C3)nc2n1-c1ccc(CN2CCNCC2)cc1 ; N#Cc1cc(N2CCN(Cc3ccc(-n4c(-c5cccnc5N)nc5ccc(N6CCCNC(=O)C6)nc54)cc3)CC2)ncn1]

A mixture of Intermediate 70 (250 mg, 502 μmol), 6-chloropyrimidine-4-carbonitrile (70.1 mg, 502 μmol) and DIEA (260 mg, 2.01 mmol) in NMP (3 mL) was taken up into a microwave tube. The sealed tube was heated at 130° C. for 1 hr under microwave. The reaction mixture was quenched with H₂O (5 mL) at 0° C., and then extracted with CH₂Cl₂ (15 mL×3). The combined organic layers were washed with aqueous NaCl (15 mL), dried over Na₂SO₄, filtered and concentrated under reduced pressure. After purified by prep-HPLC (column: Phenomenex C18 150*25 mm*10 μm; mobile phase: [water (NH₄HCO₃)-ACN]; B %: 23%-53%, over 10 min), 6-(4-(4-(2-(2-aminopyridin-3-yl)-5-(3-oxo-1,4-diazepan-1-yl)-3H-imidazo[4,5-b]pyridin-3-yl)benzyl)piperazin-1-yl)pyrimidine-4-carbonitrile (Example 284, 63.2 mg, yield: 21%) was obtained as a yellow solid. MS: m/z=601.4 [M+H]⁺. ¹H NMR (400 MHz, Dimethylsulfoxide-d₆) δ 8.56 (d, J=0.8 Hz, 1H), 7.95 (d, J=9.2 Hz, 1H), 7.93 (dd, J=4.8, 2.0 Hz, 1H), 7.57 (s, 1H), 7.46-7.42 (m, 3H), 7.35 (d, J=8.0 Hz, 2H), 7.02-6.96 (m, 3H), 6.80 (d, J=8.8 Hz, 1H), 6.30 (dd, J=7.6, 4.8 Hz, 1H), 4.18 (s, 2H), 3.83-3.80 (m, 2H), 3.77-3.67 (m, 4H), 3.61 (s, 2H), 3.20-3.16 (m, 2H), 2.48-2.45 (m, 4H), 1.67-1.62 (m, 2H).

### Example 285: 4-(8-(4-(2-(2-Aminopyridin-3-yl)-5-morpholino-3H-imidazo[4,5-b]pyridin-3-yl)benzyl)-3,8-diazabicyclo[3.2.1]octan-3-yl)-1,3,5-triazine-2-carbonitrile

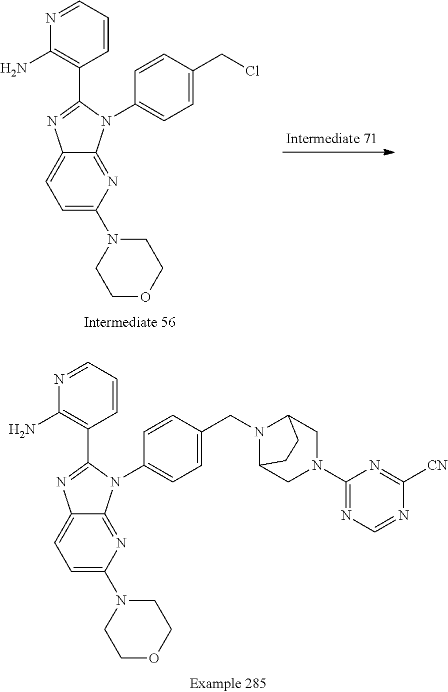 [SMILES: Nc1ncccc1-c1nc2ccc(N3CCOCC3)nc2n1-c1ccc(CCl)cc1 ; N#Cc1ncnc(N2CC3CCC(C2)N3Cc2ccc(-n3c(-c4cccnc4N)nc4ccc(N5CCOCC5)nc43)cc2)n1]

To a solution of Intermediate 56 (300 mg, 656 μmol) and Intermediate 71 (200 mg, 606 μmol, TFA salt) in CH₃CN (10 mL) were added NaI (19.7 mg, 132 μmol) and K₂CO₃ (544 mg, 3.94 mmol). The mixture was stirred at 50° C. for 5 hr. The reaction mixture was diluted with H₂O (10 mL) and extracted with CH₂Cl₂ (10 mL×2). The combined organic layers were washed with brine (10 mL×2), dried over Na₂SO₄, filtered and concentrated under reduced pressure. The residue was purified by silica gel flash chromatography (Eluent of 0˜5% MeOH in CH₂Cl₂), and then the crude product was triturated with CH₃CN (5 mL) at 25° C. for 30 min. After purified again by prep-TLC (100% EtOAc), 4-(8-(4-(2-(2-aminopyridin-3-yl)-5-morpholino-3H-imidazo[4,5-b]pyridin-3-yl)benzyl)-3,8-diazabicyclo[3.2.1]octan-3-yl)-1,3,5-triazine-2-carbonitrile (Example 285, 21.7 mg, yield: 5.2%) was obtained as a light-yellow solid. MS: m/z=601.5 [M+H]⁺. ¹H NMR (400 MHz, Dimethylsulfoxide-d₆) δ 8.71 (s, 1H), 8.02-7.94 (m, 2H), 7.53 (d, J=8.0 Hz, 2H), 7.33 (d, J=8.0 Hz, 2H), 7.04-6.98 (m, 3H), 6.90 (d, J=8.8 Hz, 1H), 6.35 (dd, J=7.6, 4.8 Hz, 1H), 4.29 (d, J=12.0 Hz, 1H), 4.18 (d, J=12.0 Hz, 1H), 3.71-3.66 (m, 4H), 3.64 (s, 2H), 3.44-3.35 (m, 6H), 3.19 (d, J=12.8 Hz, 2H), 2.03-1.96 (m, 2H), 1.54-1.43 (m, 2H).

### Example 286: 4-((1-(4-(2-(2-Aminopyridin-3-yl)-5-isopropyl-3H-imidazo[4,5-b]pyridin-3-yl)benzyl)piperidin-4-yl)amino)pyrimidine-2-carbonitrile

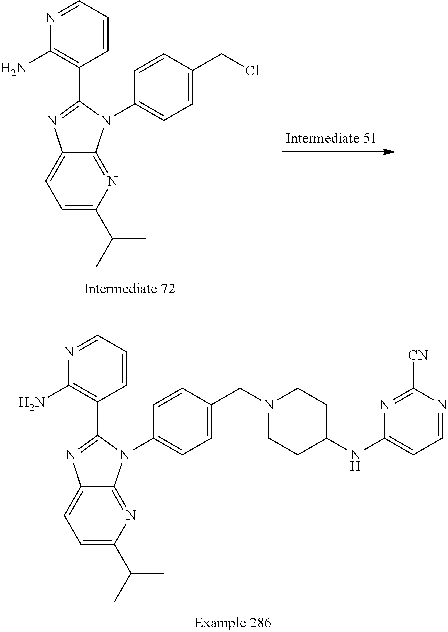 [SMILES: CC(C)c1ccc2nc(-c3cccnc3N)n(-c3ccc(CCl)cc3)c2n1 ; CC(C)c1ccc2nc(-c3cccnc3N)n(-c3ccc(CN4CCC(Nc5ccnc(C#N)n5)CC4)cc3)c2n1]

A mixture of Intermediate 72 (220 mg, 582 μmol) and Intermediate 51 (130 mg, 640 μmol) in DMF (3 mL) were added NaI (26.1 mg, 174 μmol) and K₂CO₃ (402 mg, 2.91 mmol). The mixture was stirred at 50° C. for 1 hr. The reaction mixture was diluted with H₂O (20 mL) and extracted with EtOAc (30 mL×3). The combined organic layers were washed with H₂O (30 mL×3), dried over Na₂SO₄, filtered and concentrated under reduced pressure. After purified by silica gel flash chromatography (eluent 0˜10% MeOH in CH₂Cl₂), then triturated with MeCN, 4-((1-(4-(2-(2-aminopyridin-3-yl)-5-isopropyl-3H-imidazo[4,5-b]pyridin-3-yl)benzyl)piperidin-4-yl)amino)pyrimidine-2-carbonitrile (Example 286, 71.1 mg, yield: 22%) as a yellow solid. MS: m/z=545.2 [M+H]⁺. ¹H NMR (400 MHz, Dimethylsulfoxide-d₆) δ 8.29-8.01 (m, 3H), 7.97 (dd, J=4.8, 1.6 Hz, 1H), 7.44 (d, J=8.4 Hz, 2H), 7.36 (d, J=8.4 Hz, 2H), 7.30 (d, J=8.4 Hz, 1H), 7.00 (dd, J=7.6, 1.6 Hz, 1H), 6.94 (br s, 2H), 6.67 (d, J=6.4 Hz, 1H), 6.35 (dd, J=8.0, 5.2 Hz, 1H), 3.92-3.69 (m, 1H), 3.57 (s, 2H), 3.13-2.99 (m, 1H), 2.81-2.75 (m, 2H), 2.20-2.14 (m, 2H), 1.92-1.80 (m, 2H), 1.55-1.43 (m, 2H), 1.23 (d, J=6.8 Hz, 6H).

### Example 287: 4-((1-(4-(2-(2-Aminopyridin-3-yl)-5-methoxy-3H-imidazo[4,5-b]pyridin-3-yl)benzyl)piperidin-4-yl)amino)pyrimidine-2-carbonitrile

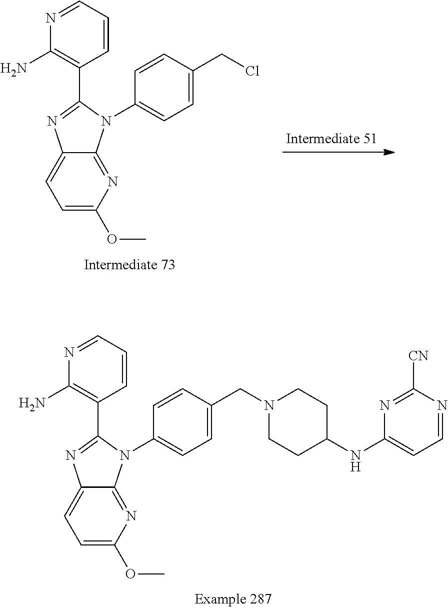 [SMILES: COc1ccc2nc(-c3cccnc3N)n(-c3ccc(CCl)cc3)c2n1 ; COc1ccc2nc(-c3cccnc3N)n(-c3ccc(CN4CCC(Nc5ccnc(C#N)n5)CC4)cc3)c2n1]

To a solution of Intermediate 73 (500 mg, 1.24 mmol) and Intermediate 51 (315 mg, 994 μmol, TFA) in DMF (4 mL) were added K₂CO₃ (859 mg, 6.21 mmol) and NaI (55.9 mg, 373 μmol). The mixture was stirred at 50° C. for 2 hr. The reaction mixture was diluted with H₂O (20 mL) and extracted with EtOAc (30 mL×3). The combined organic layers were washed with H₂O (30 mL×3), dried over Na₂SO₄, filtered and concentrated under reduced pressure. After purified by silica gel flash chromatography (Eluent of 0˜10% MeOH in CH₂Cl₂), and then triturated with MeCN (5 mL), 4-((1-(4-(2-(2-aminopyridin-3-yl)-5-methoxy-3H-imidazo[4,5-b]pyridin-3-yl)benzyl)piperidin-4-yl)amino)pyrimidine-2-carbonitrile (Example 287, 82.4 mg, yield: 12%) was obtained as a light-yellow lyophilized powder. MS: m/z=533.2 [M+H]⁺. ¹H NMR (400 MHz, Dimethylsulfoxide-d₆) δ 8.13-8.03 (m, 3H), 7.95 (dd, J=4.8, 1.2 Hz, 1H), 7.46-7.43 (m, 2H), 7.38-7.34 (m, 2H), 7.04 (dd, J=7.6, 1.6 Hz, 1H), 6.96 (br s, 2H), 6.81 (d, J=8.8 Hz, 1H), 6.67 (d, J=6.0 Hz, 1H), 6.34 (dd, 1=7.6, 4.8 Hz, 1H), 3.81-3.75 (m, 4H), 3.57 (s, 2H), 2.82-2.79 (m, 2H), 2.16-2.11 (m, 2H), 1.90-1.87 (m, 2H), 1.53-1.43 (m, 2H).

### Example 288: 2-(2-Aminopyridin-3-yl)-3-(4-((4-((2-cyanopyrimidin-4-yl)amino)piperidin-1-yl)methyl)phenyl)-3H-imidazo[4,5-b]pyridine-5-carboxamide

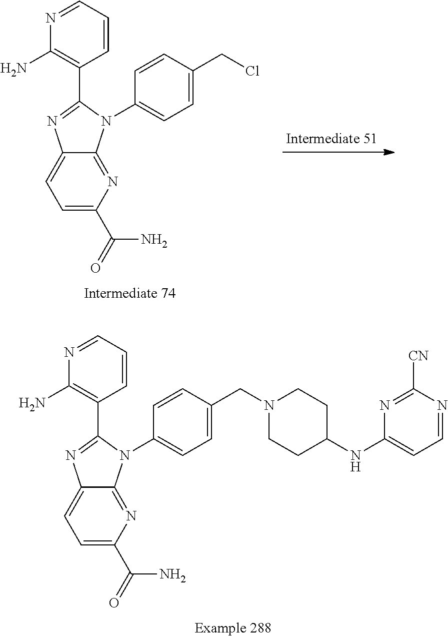 [SMILES: NC(=O)c1ccc2nc(-c3cccnc3N)n(-c3ccc(CCl)cc3)c2n1 ; N#Cc1nccc(NC2CCN(Cc3ccc(-n4c(-c5cccnc5N)nc5ccc(C(N)=O)nc54)cc3)CC2)n1]

To a solution of Intermediate 74 (400 mg, 1.06 mmol) and Intermediate 51 (335 mg, 1.06 mmol, TFA) in DMF (5 mL) were added K₂CO₃ (730 mg, 5.28 mmol) and NaI (15.8 mg, 106 μmol). The mixture was stirred at 60° C. for 1 hr. After purified by prep-HPLC (column: Waters xbridge 150*25 mm*10 μm; mobile phase: [water (NH₄HCO₃)-ACN]; gradient: 18%-38% B over 14 min), 2-(2-aminopyridin-3-yl)-3-(4-((4-((2-cyanopyrimidin-4-yl)amino)piperidin-1-yl)methyl)phenyl)-3H-imidazo[4,5-b]pyridine-5-carboxamide (Example 288, 258.3 mg, yield: 45%) was obtained as a yellow solid. MS: m/z=546.2 [M+H]⁺. ¹H NMR (400 MHz, Dimethylsulfoxide-d₆) δ 8.32 (d, J=8.4 Hz, 1H), 8.10-8.00 (m, 4H), 7.63-7.57 (m, 2H), 7.45 (br s, 4H), 7.22-7.17 (m, 1H), 6.95 (br s, 2H), 6.67 (d, J=6.0 Hz, 1H), 6.42-6.37 (m, 1H), 3.89-3.74 (m, 1H), 3.57 (s, 2H), 2.86-2.80 (m, 2H), 2.22-2.13 (m, 2H), 1.91-1.81 (m, 2H), 1.52-1.43 (m, 2H).

### Example 289: 2-(2-Aminopyridin-3-yl)-3-(4-((4-((2-cyanopyrimidin-4-yl)amino)piperidin-1-yl)methyl)phenyl)-N,N-dimethyl-3H-imidazo[4,5-b]pyridine-5-carboxamide

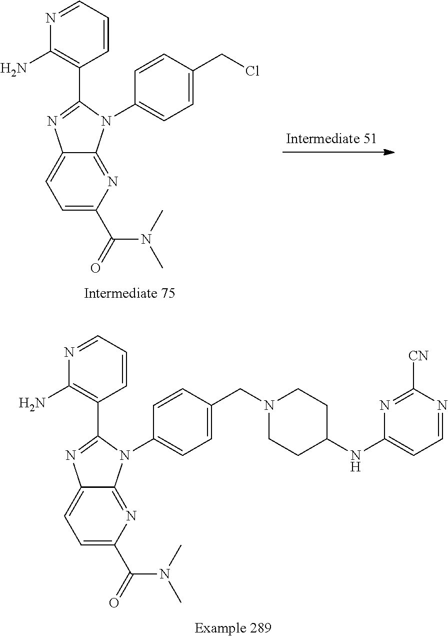 [SMILES: CN(C)C(=O)c1ccc2nc(-c3cccnc3N)n(-c3ccc(CCl)cc3)c2n1 ; CN(C)C(=O)c1ccc2nc(-c3cccnc3N)n(-c3ccc(CN4CCC(Nc5ccnc(C#N)n5)CC4)cc3)c2n1]

To a solution of Intermediate 75 (330 mg, 811 μmol) and Intermediate 51 (257 mg, 811 μmol, TFA) in DMF (4 mL) were added K₂CO₃ (336 mg, 2.43 mmol) and NaI (24.3 mg, 162 μmol). The mixture was stirred at 50° C. for 1 hr. The mixture was filtered, and the filtrate was concentrated to give the crude product. After purified by prep-HPLC (column: Waters xbridge 150*25 mm*10 μm; mobile phase: [water (NH₄HCO₃)-ACN]; gradient: 16%-46% B over 14 min), 2-(2-aminopyridin-3-yl)-3-(4-((4-((2-cyanopyrimidin-4-yl)amino)piperidin-1-yl)methyl)phenyl)-N,N-dimethyl-3H-imidazo[4,5-b]pyridine-5-carboxamide (Example 289, 204.2 mg, yield: 44%) was obtained as an off-white lyophilized powder. MS: m/z=574.4 [M+H]⁺. ¹H NMR (400 MHz, Dimethylsulfoxide-d₆) δ 8.28 (d, J=8.0 Hz, 1H), 8.12-8.03 (m, 2H), 8.00 (dd, J=4.8, 1.6 Hz, 1H), 7.58 (d, J=8.0 Hz, 1H), 7.45 (d, J=8.4 Hz, 2H), 7.40 (d, J=8.4 Hz, 2H), 7.18 (dd, J=7.6, 1.6 Hz, 1H), 6.98 (br s, 2H), 6.67 (d, J=6.0 Hz, 1H), 6.39 (dd, J=8.0, 5.2 Hz, 1H), 3.88-3.74 (m, 1H), 3.57 (s, 2H), 2.98 (s, 3H), 2.95 (s, 3H), 2.85-2.76 (m, 2H), 2.19-2.05 (m, 2H), 1.95-1.81 (m, 2H), 1.56-1.39 (m, 2H).

### Example 290: 6-(4-(4-(2-(2-Aminopyridin-3-yl)-5-cyclopropyl-3H-imidazo[4,5-b]pyridin-3-yl)benzyl)piperazin-1-yl)pyrimidine-4-carbonitrile

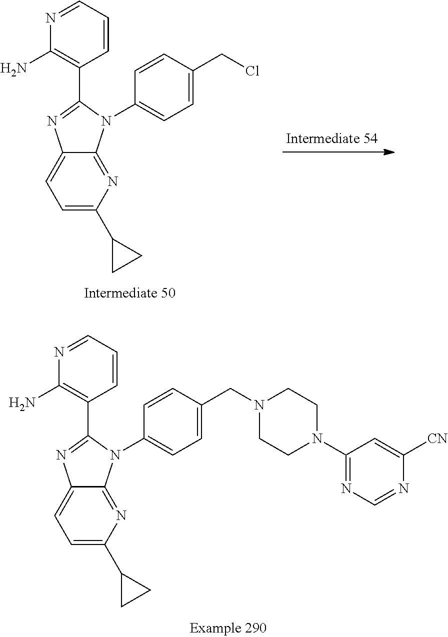 [SMILES: Nc1ncccc1-c1nc2ccc(C3CC3)nc2n1-c1ccc(CCl)cc1 ; N#Cc1cc(N2CCN(Cc3ccc(-n4c(-c5cccnc5N)nc5ccc(C6CC6)nc54)cc3)CC2)ncn1]

To a solution of Intermediate 50 (200 mg, 485 μmol) and Intermediate 54 (147 mg, 485 μmol) in DMF (2 mL) were added NaI (72.7 mg, 485 μmol) and K₂CO₃ (335 mg, 2.43 mmol). The mixture was stirred at 50° C. for 1 hr. The reaction mixture was filtered. The residue was purified by prep-HPLC (column: [water (NH₄HCO₃)-ACN]; B %: 38%-68%, 14 min) to give 6-(4-(4-(2-(2-aminopyridin-3-yl)-5-cyclopropyl-3H-imidazo[4,5-b]pyridin-3-yl)benzyl)piperazin-1-yl)pyrimidine-4-carbonitrile (Example 290, 64.4 mg, yield: 25%) as a yellow solid. MS: m/z=529.4 [M+H]⁺. ¹H NMR (400 MHz, Dimethylsulfoxide-d₆) δ 8.57 (d, J=1.2 Hz, 1H), 8.04 (d, J=8.0 Hz, 1H), 7.96 (dd, J=4.8, 2.0 Hz, 1H), 7.57 (d, J=0.8 Hz, 1H), 7.46 (d, J=8.4 Hz, 2H), 7.34 (d, J=8.4 Hz, 2H), 7.25 (d, J=8.4 Hz, 1H), 7.06 (dd, J=7.6, 1.6 Hz, 1H), 6.97 (br s, 2H), 6.32 (dd, J=7.6, 4.8 Hz, 1H), 3.84-3.66 (m, 4H), 3.62 (s, 2H), 2.49-2.45 (m, 4H), 2.21-2.13 (m, 1H), 0.96-0.89 (m, 2H), 0.85-0.79 (m, 2H).

### Example 291: 6-((1-(4-(2-(2-Aminopyridin-3-yl)-5-cyclopropyl-3H-imidazo[4,5-h]pyridin-3-yl)benzyl)piperidin-4-yl)amino)pyrimidine-4-carbonitrile

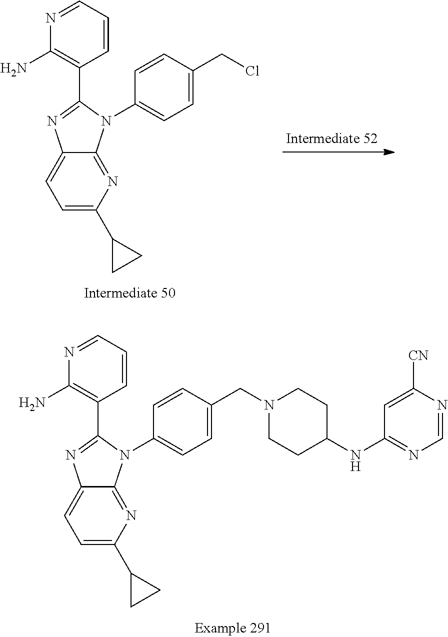 [SMILES: Nc1ncccc1-c1nc2ccc(C3CC3)nc2n1-c1ccc(CCl)cc1 ; N#Cc1cc(NC2CCN(Cc3ccc(-n4c(-c5cccnc5N)nc5ccc(C6CC6)nc54)cc3)CC2)ncn1]

To a solution of Intermediate 50 (200 mg, 485 μmol) and Intermediate 52 (154 mg, 485 μmol) in DMF (2 mL) were added NaI (36.3 mg, 242 μmol) and K₂CO₃ (335 mg, 2.43 mmol). The mixture was stirred at 50° C. for 1 hr. The reaction mixture was filtered. The residue was purified by prep-HPLC (column: [water (NH₄HCO₃)-ACN]; B %: 42%-72%, 14 min) to give 6-((1-(4-(2-(2-aminopyridin-3-yl)-5-cyclopropyl-3H-imidazo[4,5-b]pyridin-3-yl)benzyl)piperidin-4-yl)amino)pyrimidine-4-carbonitrile (Example 291, 62.3 mg, yield: 24%) was obtained as a yellow solid. MS: m/z=543.4 [M+H]⁺. ¹H NMR (400 MHz, Dimethylsulfoxide-d₆) δ 8.50 (m, 1H), 8.08 (d, J=7.2 Hz, 1H), 8.03 (d, J=8.0 Hz, 1H), 7.96 (dd, J=4.8, 1.6 Hz, 1H), 7.43 (d, J=8.4 Hz, 2H), 7.33 (d, J=8.4 Hz, 2H), 7.24 (d, J=8.4 Hz, 1H), 7.07 (dd, J=7.6, 1.8 Hz, 1H), 7.01-6.87 (m, 3H), 6.34 (dd, J=7.6, 4.8 Hz, 1H), 3.95-3.79 (m, 1H), 3.56 (s, 2H), 2.81 (d, J=11.2 Hz, 2H), 2.20-2.08 (m, 3H), 1.90 (m, 2H), 1.59-1.42 (m, 2H), 0.97-0.89 (m, 2H), 0.86-0.78 (m, 2H).

### Example 292: 4-(4-(4-(2-(2-Aminopyridin-3-yl)-5-morpholino-3H-imidazo[4,5-b]pyridin-3-yl)benzyl)piperazin-1-yl)-1,3,5-triazine-2-carbonitrile

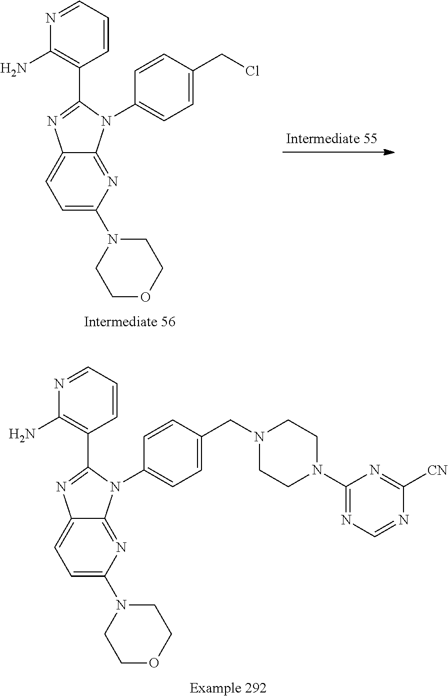 [SMILES: Nc1ncccc1-c1nc2ccc(N3CCOCC3)nc2n1-c1ccc(CCl)cc1 ; N#Cc1ncnc(N2CCN(Cc3ccc(-n4c(-c5cccnc5N)nc5ccc(N6CCOCC6)nc54)cc3)CC2)n1]

To a solution of Intermediate 56 (300 mg, 656 μmol) and Intermediate 55 (200 mg, 656 μmol) in DMF (3 mL) was added NaI (49.2 mg, 328 μmol), K₂CO₃ (453 mg, 3.28 mmol). The mixture was stirred at 25° C. for 2 hr. The mixture was quenched with H₂O (30 mL) and extracted with EtOAc (60 mL), the combined organic phase was dried over anhydrous Na₂SO₄, filtered and concentrated. The residue was purified by prep-TLC (CH₂Cl₂:MeOH=20:1) to give 4-(4-(4-(2-(2-aminopyridin-3-yl)-5-morpholino-3H-imidazo[4,5-b]pyridin-3-yl)benzyl)piperazin-1-yl)-1,3,5-triazine-2-carbonitrile (Example 292, 73.6 mg, yield: 18%) as a light-yellow solid. MS: m/z=575.4 [M+H]⁺. ¹H NMR (400 MHz, Dimethylsulfoxide-d₆) δ 8.72 (s, 1H), 7.98 (d, J=8.8 Hz, 1H), 7.95-7.91 (m, 1H), 7.46 (d, J=7.6 Hz, 2H), 7.33 (d, J=8.4 Hz, 2H), 7.03-6.97 (m, 3H), 6.90 (d, J=9.2 Hz, 1H), 6.32 (dd, J=7.2, 4.4 Hz, 1H), 3.87-3.79 (m, 4H), 3.72-3.59 (m, 8H), 3.42-3.38 (m, 4H), 3.34-3.33 (m, 2H).

### Example 293: 4-(4-(4-(2-(2-Aminopyridin-3-yl)-5-(2-cyanophenyl)-3H-imidazo[4,5-b]pyridin-3-yl)benzyl)piperazin-1-yl)pyrimidine-2-carbonitrile

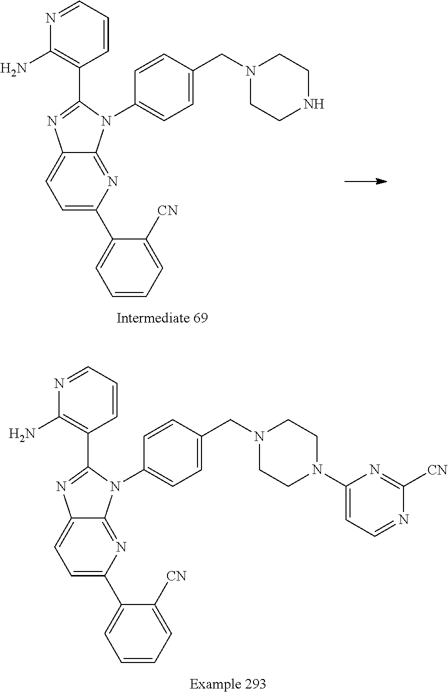 [SMILES: N#Cc1ccccc1-c1ccc2nc(-c3cccnc3N)n(-c3ccc(CN4CCNCC4)cc3)c2n1 ; N#Cc1nccc(N2CCN(Cc3ccc(-n4c(-c5cccnc5N)nc5ccc(-c6ccccc6C#N)nc54)cc3)CC2)n1]

A mixture of Intermediate 69 (100 mg, 206 μmol), 4-chloropyrimidine-2-carbonitrile (29 mg, 206 μmol) and DIEA (133 mg, 1.03 mmol) in NMP (3 mL) was taken up into a microwave tube. The sealed tube was heated at 130° C. for 1 hr under microwave. The reaction mixture was quenched with H₂O (5 mL) at 0° C., and then diluted with CH₂Cl₂ (10 mL) and extracted with CH₂Cl₂ (15 mL×2). The combined organic layers were washed with brine (15 mL), dried over Na₂SO₄, filtered and concentrated under reduced pressure. After purified by prep-HPLC (column: Waters xbridge 150*25 mm 10 μm; mobile phase: [water (NH₄HCO₃)-ACN]; gradient: 33%-63% B over 14 min), 4-(4-(4-(2-(2-aminopyridin-3-yl)-5-(2-cyanophenyl)-3H-imidazo[4,5-b]pyridin-3-yl)benzyl)piperazin-1-yl)pyrimidine-2-carbonitrile (Example 293, 35.1 mg, yield: 29%) was obtained as a yellow powder. MS: m/z=590.2 [M+H]⁺. ¹H NMR (400 MHz, Dimethylsulfoxide-d₆) δ 8.38 (d, J=8.0 Hz, 1H), 8.26 (d, J=6.4 Hz, 1H), 8.01 (dd, J=4.8, 2.0 Hz, 1H), 7.95 (d, J=8.0 Hz, 1H), 7.92-7.77 (m, 3H), 7.67-7.57 (m, 1H), 7.49-7.43 (m, 4H), 7.19 (dd, J=7.6, 2.0 Hz, 1H), 7.10 (d, J=6.8 Hz, 1H), 7.00 (br s, 2H), 6.39 (dd, J=7.6, 4.8 Hz, 1H), 3.68-3.67 (m, 4H), 3.61 (s, 2H), 2.49-2.45 (m, 4H).

### Example 294: 4-(4-(4-(2-(2-Aminopyridin-3-yl)-5-(4-cyanophenyl)-3H-imidazo[4,5-b]pyridin-3-yl)benzyl)piperazin-1-yl)pyrimidine-2-carbonitrile

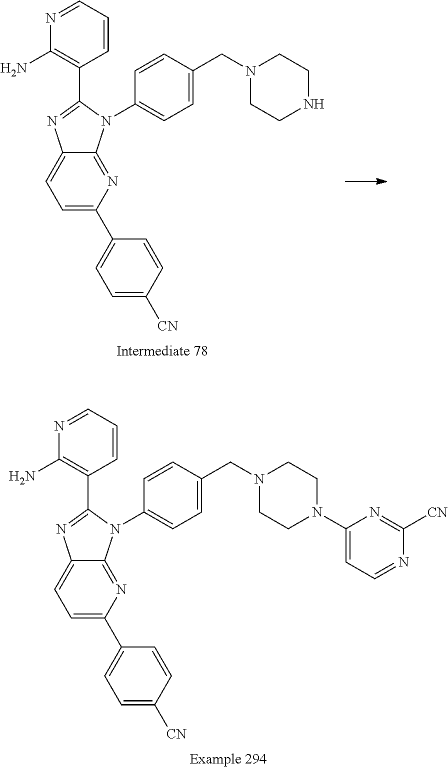 [SMILES: N#Cc1ccc(-c2ccc3nc(-c4cccnc4N)n(-c4ccc(CN5CCNCC5)cc4)c3n2)cc1 ; N#Cc1ccc(-c2ccc3nc(-c4cccnc4N)n(-c4ccc(CN5CCN(c6ccnc(C#N)n6)CC5)cc4)c3n2)cc1]

A mixture of Intermediate 78 (100 mg, 206 μmol), 4-chloropyrimidine-2-carbonitrile (29 mg, 210 μmol) and DIEA (136 mg, 1.05 mmol) in NMP (3 mL) was taken up into a microwave tube. The sealed tube was heated at 130° C. for 1 hr under microwave. The reaction mixture was quenched with H₂O (5 mL) at 0° C., and then extracted with CH₂Cl₂ (15 mL×2). The combined organic layers were washed with brine (15 mL), dried over Na₂SO₄, filtered and concentrated under reduced pressure. After purified byprep-HPLC (column: Waters xbridge 150*25 mm 10 μm; mobile phase: [water (NH₄HCO₃)-ACN]; gradient: 45%-75% B over 10 min), 4-(4-(4-(2-(2-aminopyridin-3-yl)-5-(4-cyanophenyl)-3H-imidazo[4,5-b]pyridin-3-yl)benzyl)piperazin-1-yl)pyrimidine-2-carbonitrile (Example 294, 13.6 mg, yield: 10%) was obtained as a yellow powder. MS: m/z=590.3 [M+H]⁺. ¹H NMR (400 MHz, Methanol-d₄) δ 8.25 (dd, J=8.4, 2.0 Hz, 3H), 8.18 (d, J=6.8 Hz, 1H), 8.06 (d, J=8.4 Hz, 1H), 8.00 (dd, J=5.2, 2.0 Hz, 1H), 7.80 (d, J=8.8 Hz, 2H), 7.58 (d, J=8.8 Hz, 2H), 7.47 (d, J=8.4 Hz, 2H), 7.34 (dd, J=7.6, 2.0 Hz, 1H), 6.95 (d, J=6.4 Hz, 1H), 6.48 (dd, J=7.6, 5.2 Hz, 1H), 3.83-3.74 (m, 4H), 3.70 (s, 2H), 2.63-2.57 (m, 4H).

### Example 295: 4-(4-(4-(2-(2-Aminopyridin-3-yl)-5-(3-fluorophenyl)-3H-imidazo[4,5-b]pyridin-3-yl)benzyl)piperazin-1-yl)pyrimidine-2-carbonitrile

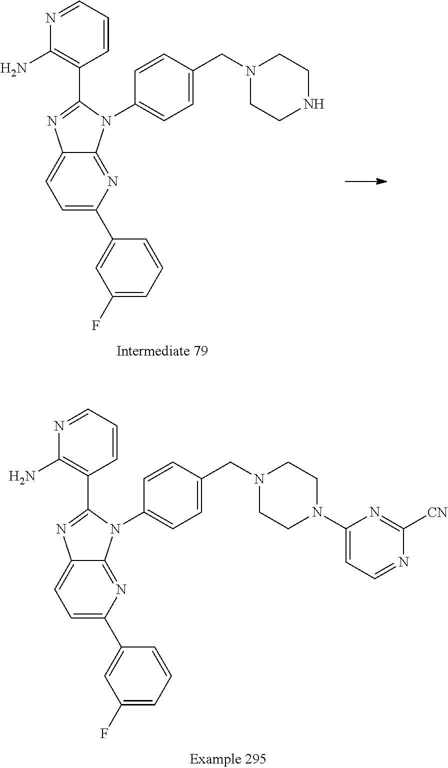 [SMILES: Nc1ncccc1-c1nc2ccc(-c3cccc(F)c3)nc2n1-c1ccc(CN2CCNCC2)cc1 ; N#Cc1nccc(N2CCN(Cc3ccc(-n4c(-c5cccnc5N)nc5ccc(-c6cccc(F)c6)nc54)cc3)CC2)n1]

A mixture of Intermediate 79 (100 mg, 209 μmol), 4-chloropyrimidine-2-carbonitrile (29 mg, 210 μmol) and DIEA (136 mg, 1.05 mmol) in NMP (3 mL) was taken up into a microwave tube. The sealed tube was heated at 130° C. for 1 hr under microwave. The reaction mixture was quenched with H₂O (5 mL) at 0° C., and then extracted with CH₂Cl₂ (15 mL×3). The combined organic layers were washed with brine (15 mL), dried over Na₂SO₄, filtered and concentrated under reduced pressure. After purified by prep-HPLC (column: Waters xbridge 150*25 mm 10 μm; mobile phase: [water (NH₄HCO₃)-ACN]; gradient: 44%-74% B over 14 min), 4-(4-(4-(2-(2-aminopyridin-3-yl)-5-(3-fluorophenyl)-3H-imidazo[4,5-b]pyridin-3-yl)benzyl)piperazin-1-yl)pyrimidine-2-carbonitrile (Example 295, 43 mg, yield: 33%) was obtained as a yellow powder. MS: m/z=583.3 [M+H]⁺. ¹H NMR (400 MHz, Methanol-d₄) δ 8.23-8.16 (m, 2H), 8.01-7.95 (m, 2H), 7.87 (d, J=8.4 Hz, 1H), 7.81-7.76 (m, 1H), 7.59 (d, J=8.4 Hz, 2H), 7.49-7.44 (m, 3H), 7.34 (dd, J=7.6, 4.6 Hz, 1H), 7.13-7.09 (m, 1H), 6.95 (d, J=6.4 Hz, 1H), 6.48 (dd, J=7.6, 5.2 Hz, 1H), 3.78-3.76 (m, 4H), 3.70 (s, 2H), 2.61 (t, J=4.8 Hz, 4H). ¹⁹F NMR (400 MHz, Methanol-d₄) δ −115.26.

### Example 296: 4-(4-(4-(2-(2-Aminopyridin-3-yl)-5-(3-chlorophenyl)-3H-imidazo[4,5-b]pyridin-3-yl)benzyl)piperazin-1-yl)pyrimidine-2-carbonitrile

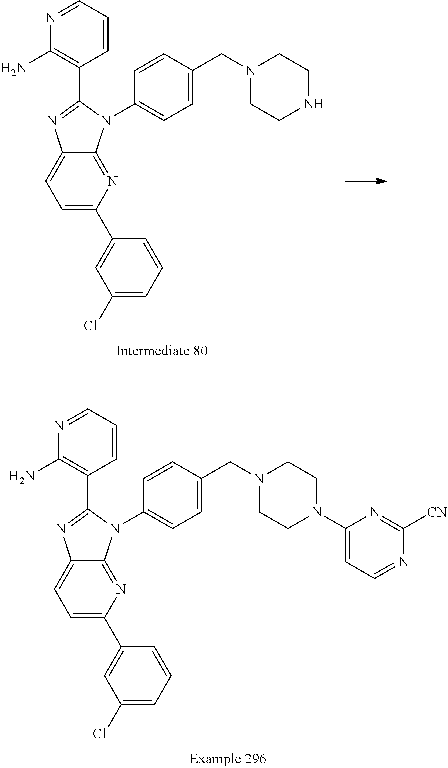 [SMILES: Nc1ncccc1-c1nc2ccc(-c3cccc(Cl)c3)nc2n1-c1ccc(CN2CCNCC2)cc1 ; N#Cc1nccc(N2CCN(Cc3ccc(-n4c(-c5cccnc5N)nc5ccc(-c6cccc(Cl)c6)nc54)cc3)CC2)n1]

A mixture of Intermediate 80 (100 mg, 202 μmol), 4-chloropyrimidine-2-carbonitrile (28 mg, 202 μmol) and DIEA (130 mg, 1.01 mmol) in NMP (3 mL) was taken up into a microwave tube. The sealed tube was heated at 130° C. for 1 hr under microwave. The reaction mixture was quenched with H₂O (5 mL) at 0° C., and then extracted with CH₂Cl₂ (15 mL×3). The combined organic layers were washed with brine (15 mL), dried over Na₂SO₄, filtered and concentrated under reduced pressure. After purified by prep-HPLC (column: Phenomenex C18 150×25 mm×10 μm; mobile phase: [water (NH₄HCO₃)-ACN]; gradient. 55%-85% B over 10 min), 4-(4-(4-(2-(2-Aminopyridin-3-yl)-5-(3-chlorophenyl)-3H-imidazo[4,5-b]pyridin-3-yl)benzyl)piperazin-1-yl)pyrimidine-2-carbonitrile (Example 296, 45.7 mg, yield: 36%) was obtained as a brown solid. MS: m/z=599.2 [M+H]⁺. ¹H NMR (400 MHz, Methanol-d₄) δ 8.23-8.16 (m, 2H), 8.05 (s, 1H), 8.00-7.95 (m, 3H), 7.59 (d, J=8.4 Hz, 2H), 7.47 (d, J=8.4 Hz, 2H), 7.46-7.36 (m, 2H), 7.34 (dd, J=7.6, 1.6 Hz, 1H), 6.94 (d, J=6.4 Hz, 1H), 6.48 (dd, J=7.6, 4.8 Hz, 1H), 3.73-3.80 (m, 4H), 3.70 (s, 2H), 2.63-2.59 (m, 4H).

### Example 297: 2-(4-(4-(2-(2-Aminopyridin-3-yl)-5-(3-chlorophenyl)-3H-imidazo[4,5-b]pyridin-3-yl)benzyl)piperazin-1-yl)pyrimidine-4-carbonitrile

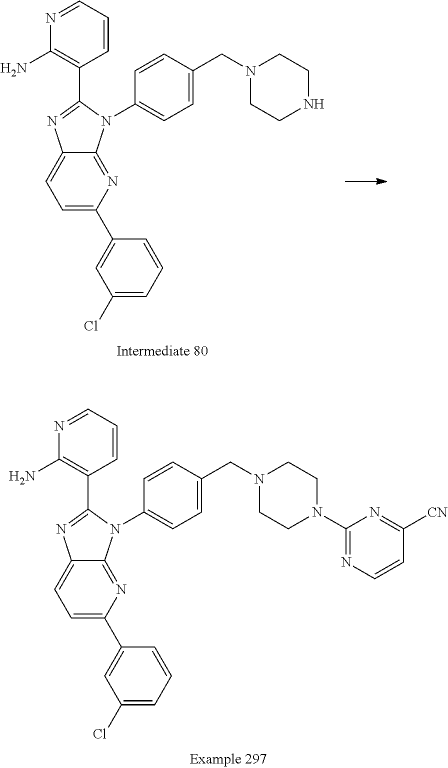 [SMILES: Nc1ncccc1-c1nc2ccc(-c3cccc(Cl)c3)nc2n1-c1ccc(CN2CCNCC2)cc1 ; N#Cc1ccnc(N2CCN(Cc3ccc(-n4c(-c5cccnc5N)nc5ccc(-c6cccc(Cl)c6)nc54)cc3)CC2)n1]

To a solution of Intermediate 80 (100 mg, 202 μmol) and 2-chloropyrimidine-4-carbonitrile (28 mg, 202 μmol) and DIEA (130 mg, 1.01 mmol) in NMP (3 mL) was taken up into a microwave tube. The sealed tube was heated at 130° C. for 1 hr under microwave. The reaction mixture was quenched with H₂O (5 mL) at 0° C., and then diluted with CH₂Cl₂ (10 mL) and extracted with CH₂Cl₂ (15 mL×2). The combined organic layers were washed with brine (15 mL), dried over Na₂SO₄, filtered and concentrated under reduced pressure. After purified by prep-HPLC (column: Waters xbridge 150*25 mm 10 μm; mobile phase: [water (NH₄HCO₃)-ACN]; gradient: 60%-80% B over 14 min), 2-(4-(4-(2-(2-Aminopyridin-3-yl)-5-(3-chlorophenyl)-3H-imidazo[4,5-b]pyridin-3-yl)benzyl)piperazin-1-yl)pyrimidine-4-carbonitrile (Example 297, 79 mg, yield: 65%) was obtained as a yellow lyophilized powder. MS: m/z=599.4 [M+H]⁺. ¹H NMR (400 MHz, Dimethylsulfoxide-d₆) δ 8.65 (d, J=4.8 Hz, 1H), 8.29 (d, J=8.4 Hz, 1H), 8.07-8.00 (m, 4H), 7.54-7.45 (m, 6H), 7.19-7.14 (m, 2H), 7.05 (br s, 2H), 6.38 (dd, J=7.2, 5.2 Hz, 1H), 3.79-3.77 (m, 4H), 3.65 (s, 2H), 2.47-2.35 (m, 4H).

### Example 298: 4-((1-(4-(2-(2-Aminopyridin-3-yl)-5-cyclopropyl-3H-imidazo[4,5-b]pyridin-3-yl)benzyl)piperidin-4-yl)amino)pyrimidine-2-carbonitrile

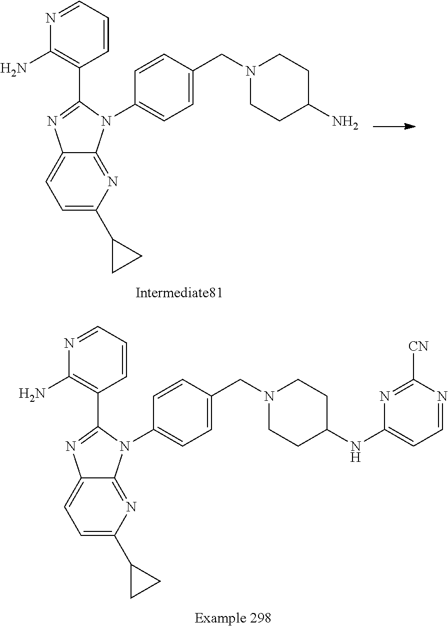 [SMILES: Nc1ncccc1-c1nc2ccc(C3CC3)nc2n1-c1ccc(CN2CCC(N)CC2)cc1 ; N#Cc1nccc(NC2CCN(Cc3ccc(-n4c(-c5cccnc5N)nc5ccc(C6CC6)nc54)cc3)CC2)n1]

A mixture of Intermediate 81 (110 mg, 250 μmol), 4-chloropyrimidine-2-carbonitrile (38 mg, 275 μmol) and DIEA (97 mg, 751 μmol) in NMP (2 mL) was taken up into a microwave tube. The sealed tube was stirred at 130° C. for 30 min under microwave. The reaction mixture was filtered. The residue was purified by prep-HPLC (column: Waters xbridge 150*25 mm 10 μm; mobile phase: [water (NH₄HCO₃)-ACN]; B %: 33%-63%, 14 min), 4-((1-(4-(2-(2-aminopyridin-3-yl)-5-cyclopropyl-3H-imidazo[4,5-b]pyridin-3-yl)benzyl)piperidin-4-yl)amino)pyrimidine-2-carbonitrile (Example 298, 19.4 mg, yield: 14%) was obtained as a light-pink solid. MS: m/z=543.3 [M+H]⁺. ¹H NMR (400 MHz, Dimethylsulfoxide-d₆) δ 8.10-8.01 (m, 3H), 7.96 (dd, J=4.8, 1.6 Hz, 1H), 7.43 (d, J=8.0 Hz, 2H), 7.33 (d, =8.0 Hz, 2H), 7.24 (d, J=8.0 Hz, 1H), 7.06 (dd, J=7.6, 1.6 Hz, 1H), 6.98 (br s, 2H), 6.67 (d, J=6.4 Hz, 1H), 6.34 (dd, J=7.6, 4.8 Hz, 1H), 3.88-3.75 (m, 1H), 3.57 (s, 2H), 2.85-2.78 (m, 2H), 2.20-2.11 (m, 3H), 1.93-1.89 (m, 2H), 1.56-1.45 (m, 2H), 0.96-0.90 (m, 2H), 0.85-0.80 (m, 2H).

### Example 299: 4-((1-(4-(2-(2-Aminopyridin-3-yl)-5-(tetrahydro-2H-pyran-4-yl)-3H-imidazo[4,5-b]pyridin-3-yl)benzyl)piperidin-4-yl)amino)pyrimidine-2-carbonitrile

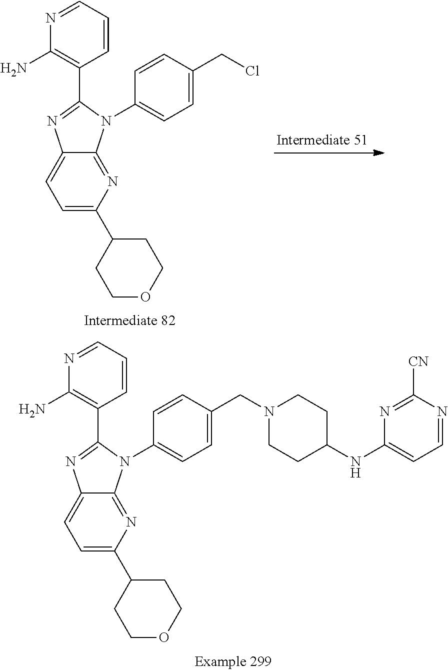 [SMILES: Nc1ncccc1-c1nc2ccc(C3CCOCC3)nc2n1-c1ccc(CCl)cc1 ; N#Cc1nccc(NC2CCN(Cc3ccc(-n4c(-c5cccnc5N)nc5ccc(C6CCOCC6)nc54)cc3)CC2)n1]

To a solution of Intermediate 82 (300 mg, 714 μmol) and Intermediate 51 (174 mg, 857 μmol) in DMF (3 mL) were added NaI (21 mg, 143 μmol) and K₂CO₃ (296 mg, 2.1 mmol). The mixture was stirred at 50° C. for 2 hr. Then the mixture was diluted with H₂O (20 mL) and extracted with EtOAc (20 mL×2). The combined organic layers were washed with brine (20 mL), dried over Na₂SO₄, filtered and concentrated under reduced pressure. The crude product was purified by silica gel flash chromatography (Eluent of 0˜6% MeOH in CH₂Cl₂) to give 4-((1-(4-(2-(2-aminopyridin-3-yl)-5-(tetrahydro-2H-pyran-4-yl)-3H-imidazo[4,5-b]pyridin-3-yl)benzyl)piperidin-4-yl)amino)pyrimidine-2-carbonitrile (Example 299, 44.2 mg, yield: 10%) as a light yellow solid. MS: m/z=587.2 [M+H]⁺. ¹H NMR (400 MHz, Dimethylsulfoxide-d₆) δ 8.16-8.01 (m, 3H), 7.97 (dd, J=4.8, 1.6 Hz, 1H), 7.47-7.42 (m, 2H), 7.39-7.30 (m, 3H), 7.09 (dd, J=8.0, 1.6 Hz, 1H), 6.94 (br s, 2H), 6.67 (d, J=6.0 Hz, 1H), 6.35 (dd, J=7.6, 4.8, Hz, 1H), 3.96-3.88 (m, 2H), 3.87-3.75 (m, 1H), 3.57 (s, 2H), 3.47-3.39 (m, 2H), 3.04-2.94 (m, 1H), 2.86-2.77 (m, 2H), 2.21-2.08 (m, 2H), 1.95-1.83 (m, 2H), 1.79-1.71 (m, 4H), 1.55-1.43 (m, 2H).

### Example 300: 4-((1-(4-(2-(2-Aminopyridin-3-yl)-5-phenyl-3H-imidazo[4,5-b]pyridin-3-yl)benzyl)azetidin-3-yl)amino)-1,3,5-triazine-2-carbonitrile

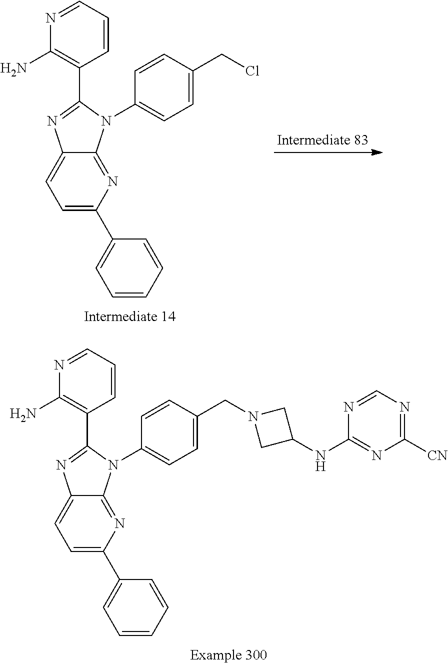 [SMILES: Nc1ncccc1-c1nc2ccc(-c3ccccc3)nc2n1-c1ccc(CCl)cc1 ; N#Cc1ncnc(NC2CN(Cc3ccc(-n4c(-c5cccnc5N)nc5ccc(-c6ccccc6)nc54)cc3)C2)n1]

To a solution of Intermediate 14 (180 mg, HCl salt, 401 μmol) and Intermediate 83 (116 mg, TFA salt, 401 μmol) in DMF (2 mL) were added NaI (30.1 mg, 200 μmol) and K₂CO₃ (277 mg, 2.01 mmol). The mixture was stirred at 50° C. for 2 hr. The mixture was concentrated under reduced pressure. The residue was purified by prep-HPLC (column: [water (NH₄HCO₃)-ACN]; B %: 39%-69%, 14 min) give 4-((1-(4-(2-(2-aminopyridin-3-yl)-5-phenyl-3H-imidazo[4,5-b]pyridin-3-yl)benzyl)azetidin-3-yl)amino)-1,3,5-triazine-2-carbonitrile (Example 300, 50 mg, yield: 14%) as a yellow solid. MS: m/z=552.4 [M+H]⁺. ¹H NMR (400 MHz, Dimethylsulfoxide-d₆) δ 9.40 (d, J=6.4 Hz, 1H), 8.76-8.63 (m, 1H), 8.27 (d, J=8.4 Hz, 1H), 8.05-7.96 (m, 4H), 7.49-7.37 (m, 7H), 7.16 (dd, J=7.6, 2.0 Hz, 1H), 7.00 (br s, 2H), 6.39 (dd, J=7.6, 4.8 Hz, 1H), 4.51-4.36 (m, 1H), 3.70 (s, 2H), 3.61 (t, J=7.2 Hz, 2H), 3.15-3.05 (m, 2H).

### Example 301: 4-(6-(4-(2-(2-Aminopyridin-3-yl)-5-phenyl-3H-imidazo[4,5-b]pyridin-3-yl)benzyl)-2,6-diazaspiro[3.4]octan-2-yl)-1,3,5-triazine-2-carbonitrile

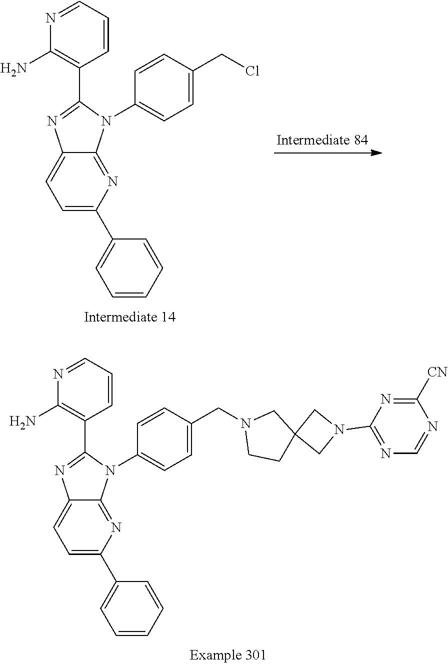 [SMILES: Nc1ncccc1-c1nc2ccc(-c3ccccc3)nc2n1-c1ccc(CCl)cc1 ; N#Cc1ncnc(N2CC3(CCN(Cc4ccc(-n5c(-c6cccnc6N)nc6ccc(-c7ccccc7)nc65)cc4)C3)C2)n1]

To a solution of Intermediate 14 (200 mg, 486 μmol) in DMF (8 mL) were added Intermediate 84 (115 mg, 534 μmol), NaI (15 mg, 97 μmol) and K₂CO₃ (201 mg, 1.46 mmol) at 25° C., the mixture was stirred at 50° C. for 3 hr. The reaction mixture was poured into H₂O (50 mL). The mixture was extracted with CH₂Cl₂ (20 mL×2). The combined organic layers were washed with brine (40 mL), dried over anhydrous Na₂SO₄, filtered and concentrated to dryness. The residue was purified by silica gel flash chromatography (Eluent of 0˜8% MeOH in CH₂Cl₂) to give 4-(6-(4-(2-(2-aminopyridin-3-yl)-5-phenyl-3H-imidazo[4,5-b]pyridin-3-yl)benzyl)-2,6-diazaspiro[3.4]octan-2-yl)-1,3,5-triazine-2-carbonitrile (Example 301, 19.3 mg, yield: 6%) as a yellow solid. MS: m/z=592.3 [M+H]⁺. ¹H NMR (400 MHz, Dimethylsulfoxide-d₆) δ 8.67 (s, 1H), 8.27 (d, J=8.4 Hz, 1H), 8.06-7.96 (m, 4H), 7.52-7.43 (m, 6H), 7.41-7.36 (m, 1H), 7.17 (dd, J=8.0, 2.0 Hz, 1H), 7.02 (br s, 2H), 6.38 (dd, J=7.6, 4.8 Hz, 1H), 4.12 (s, 4H), 3.69 (s, 2H), 2.78 (s, 2H), 2.62-2.58 (m, 2H), 2.16-2.10 (m, 2H).

### Example 302: 4-(7-(4-(2-(2-Aminopyridin-3-yl)-5-phenyl-3H-imidazo[4,5-b]pyridin-3-yl)benzyl)-2,7-diazaspiro[4.4]nonan-2-yl)pyrimidine-2-carbonitrile

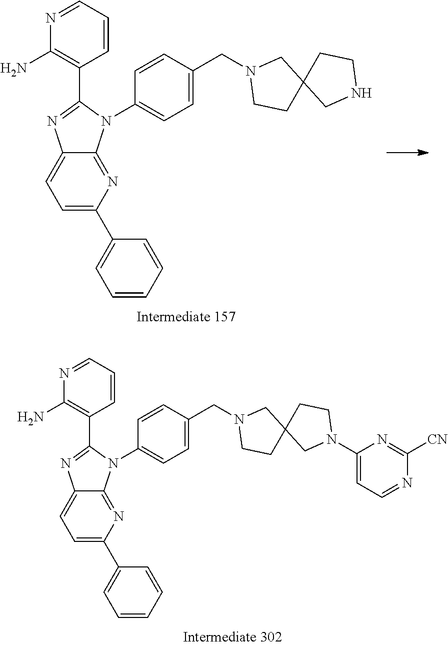 [SMILES: Nc1ncccc1-c1nc2ccc(-c3ccccc3)nc2n1-c1ccc(CN2CCC3(CCNC3)C2)cc1 ; N#Cc1nccc(N2CCC3(CCN(Cc4ccc(-n5c(-c6cccnc6N)nc6ccc(-c7ccccc7)nc65)cc4)C3)C2)n1]

To a solution of Intermediate 157 (200 mg, 372 μmol) and 4-chloropyrimidine-2-carbonitrile (62 mg, 446 μmol) in DMF (1 mL) were added K₂CO₃ (257 mg, 2.0 mmol) and NaI (56 mg, 372 μmol). The mixture was stirred at 80° C. for 1 hr. The reaction mixture was diluted with H₂O (20 mL) and extracted with EtOAc (40 mL). The combined organic layers were washed with brine (30 mL), dried over Na₂SO₄, filtered and concentrated under reduced pressure. The residue was purified by prep-HPLC (column: Phenomenex C18 150*25 mm*10 um; mobile phase: [water (NH₄HCO₃)-ACN]; gradient: B: 50%-80%, 14 min) to give 4-(7-(4-(2-(2-aminopyridin-3-yl)-5-phenyl-3H-imidazo[4,5-b]pyridin-3-yl)benzyl)-2,7-diazaspiro[4.4]nonan-2-yl)pyrimidine-2-carbonitrile (Example 302, 11 mg, yield: 5%) as an off-white solid. MS: m/z=605.3 [M+H]⁺. ¹H NMR (400 MHz, Methanol-d₄) δ 8.19 (d, J=8.4 Hz, 1H), 8.14-8.10 (m, 1H), 8.02 (d, J=7.2 Hz, 2H), 7.97-7.91 (m, 2H), 7.56 (d, J=8.4 Hz, 2H), 7.47-7.37 (m, 5H), 7.34-7.28 (m, 1H), 6.64 (d, J=6.4 Hz, 1H), 6.46-6.39 (m, 1H), 3.76 (s, 2H), 3.67-3.54 (m, 2H), 3.47-3.41 (m, 2H), 2.88-2.81 (m, 1H), 2.77-2.66 (m, 2H), 2.65-2.55 (m, 1H), 2.15-2.03 (m, 2H), 1.98-1.90 (m, 2H).

### Example 303: 4-(6-(4-(2-(2-Aminopyridin-3-yl)-5-phenyl-3H-imidazo[4,5-b]pyridin-3-yl)benzyl)-2,6-diazaspiro[3.3]heptan-2-yl)-1,3,5-triazine-2-carbonitrile

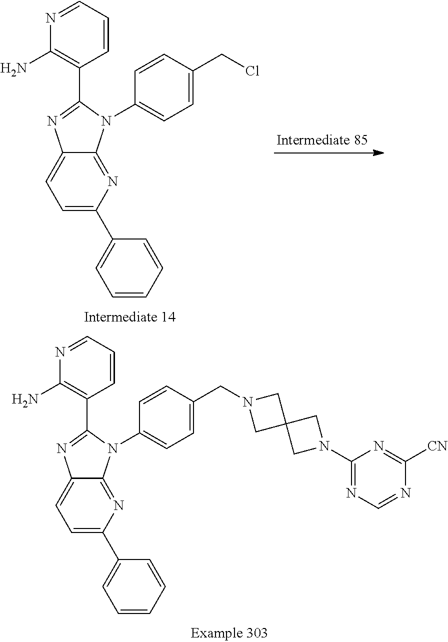 [SMILES: Nc1ncccc1-c1nc2ccc(-c3ccccc3)nc2n1-c1ccc(CCl)cc1 ; N#Cc1ncnc(N2CC3(CN(Cc4ccc(-n5c(-c6cccnc6N)nc6ccc(-c7ccccc7)nc65)cc4)C3)C2)n1]

To a solution of Intermediate 14 (200 mg, 486 μmol) in DMF (8 mL) were added Intermediate 85 (108 mg, 534 μmol), NaI (15 mg, 97 μmol) and K₂CO₃ (201 mg, 1.46 mmol) at 25° C. The mixture was stirred at 50° C. for 3 hr. The reaction mixture was poured into H₂O (50 mL). The mixture was extracted with CH₂Cl₂ (20 mL×2). The combined organic layers were washed with brine (40 mL), dried over anhydrous Na₂SO₄, filtered and concentrated to dryness. The residue was purified by silica gel flash chromatography (Eluent of 0-8% MeOH in CH₂Cl₂) to give 4-(6-(4-(2-(2-aminopyridin-3-yl)-5-phenyl-3H-imidazo[4,5-b]pyridin-3-yl)benzyl)-2,6-diazaspiro[3.3]heptan-2-yl)-1,3,5-triazine-2-carbonitrile (Example 303, 17.6 mg, yield: 6%) as a white solid. MS: m/z=578.3 [M+H]⁺. ¹H NMR (400 MHz, Dimethylsulfoxide-d₆) δ 8.67 (s, 1H), 8.27 (d, J=8.4 Hz, 1H), 8.07-7.97 (m, 4H), 7.49-7.39 (m, 711), 7.15 (dd, J=7.6, 1.6 Hz, 1H), 7.00 (br s, 2H), 6.44-6.38 (m, 1H), 4.28 (s, 4H), 3.64 (s, 2H), 3.40 (s, 4H).

### Example 304: 4-(4-(4-(2-(2-Aminopyridin-3-yl)-5-(2-chlorophenyl)-3H-imidazo[4,5-b]pyridin-3-yl)benzyl)piperazin-1-yl)-1,3,5-triazine-2-carbonitrile

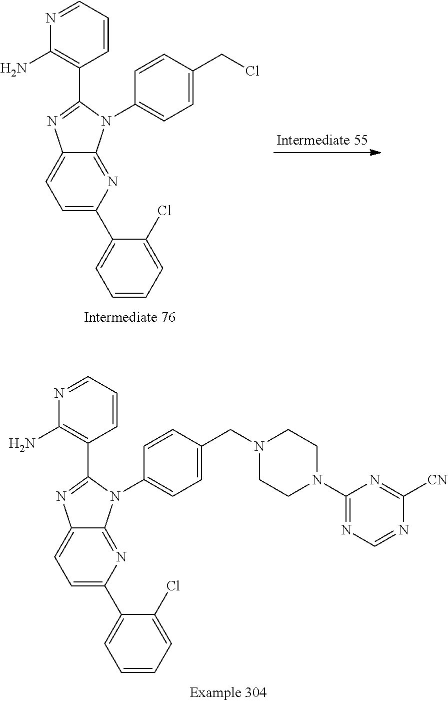 [SMILES: Nc1ncccc1-c1nc2ccc(-c3ccccc3Cl)nc2n1-c1ccc(CCl)cc1 ; N#Cc1ncnc(N2CCN(Cc3ccc(-n4c(-c5cccnc5N)nc5ccc(-c6ccccc6Cl)nc54)cc3)CC2)n1]

To a solution of Intermediate 76 (300 mg, 621 μmol) and Intermediate 55 (189 mg, 621 mol) in DMF (2 mL) were added NaI (46.6 mg, 310 μmol) and K₂CO₃ (429 mg, 3.11 mmol). The mixture was stirred at 50° C. for 2 hr. The reaction mixture was filtered and the residue was purified by prep-HPLC (column: [water (NH₄HCO₃)-ACN]; B %: 47%-77%, 14 min) to give 4-(4-(4-(2-(2-aminopyridin-3-yl)-5-(2-chlorophenyl)-3H-imidazo[4,5-b]pyridin-3-yl)benzyl)piperazin-1-yl)-1,3,5-triazine-2-carbonitrile (Example 304, 102 mg, yield: 25%) as a yellow solid. MS: m/z=600.4 [M+H]⁺. ¹H NMR (400 MHz, Dimethylsulfoxide-d₆) δ 8.71 (s, 1H), 8.29 (d, J=8.0 Hz, 1H), 8.02-7.98 (m, 1H), 7.63 (d, J=8.4 Hz, 1H), 7.58-7.53 (m, 2H), 7.47-7.43 (m, 6H), 7.16 (dd, J=7.6, 1.2 Hz, 1H), 6.95 (br s, 2H), 6.39 (dd, J=7.6, 4.8 Hz, 1H), 3.85-3.77 (m, 4H), 3.60 (s, 2H), 2.60-2.54 (m, 4H).

### Example 305: 4-((1-(4-(2-(2-Aminopyridin-3-yl)-5-(2-chlorophenyl)-3H-imidazo[4,5-b]pyridin-3-yl)benzyl)piperidin-4-yl)amino)pyrimidine-2-carbonitrile

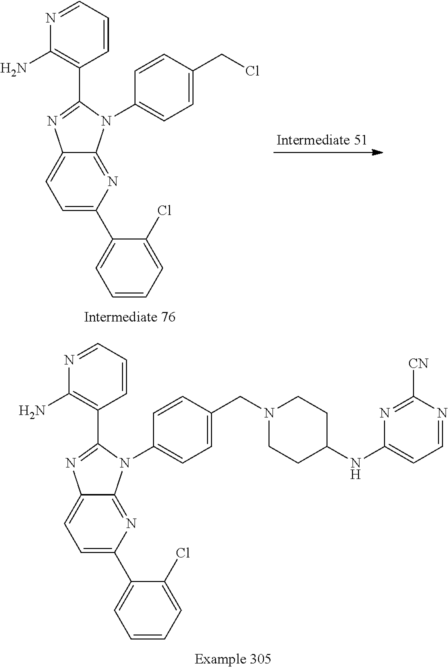 [SMILES: Nc1ncccc1-c1nc2ccc(-c3ccccc3Cl)nc2n1-c1ccc(CCl)cc1 ; N#Cc1nccc(NC2CCN(Cc3ccc(-n4c(-c5cccnc5N)nc5ccc(-c6ccccc6Cl)nc54)cc3)CC2)n1]

To a solution of Intermediate 76 (250 mg HCl salt, 517 μmol) and Intermediate 51 (164 mg TFA salt, 517 μmol) in DMF (2 mL) were added NaI (15.5 mg, 103 μmol) and K₂CO₃ (357 mg, 2.59 mmol). The mixture was stirred at 50° C. for 1 hr. The reaction mixture was diluted with water (10 mL) and extracted with EtOAc (10 mL×2). The combined organic layers were washed with brine (10 mL), dried over Na₂SO₄, filtered and concentrated under reduced pressure. The residue was purified by silica gel flash chromatography (Eluent of 0˜7% MeOH in CH₂Cl₂) to give 4-((1-(4-(2-(2-aminopyridin-3-yl)-5-(2-chlorophenyl)-3H-imidazo[4,5-b]pyridin-3-yl)benzyl)piperidin-4-yl)amino)pyrimidine-2-carbonitrile (Example 305, 45.4 mg, yield: 14%,) as a yellow solid. MS: m/z=613.4 [M+H]⁺. ¹H NMR (400 MHz, Dimethylsulfoxide-d₆) δ 8.29 (d, J=8.4 Hz, 1H), 8.11-7.98 (m, 3H), 7.63 (d, J=8.0 Hz, 1H), 7.59-7.52 (m, 2H), 7.46-7.40 (m, 6H), 7.16 (d, J=7.2 Hz, 1H), 6.97 (br s, 2H), 6.66 (d, J=6.0 Hz, 1H), 6.38 (dd, J=7.6, 4.8 Hz, 1H), 3.86-3.72 (m, 1H), 3.54 (s, 2H), 2.79 (m, 2H), 2.19-2.06 (m, 2H), 1.86 (m, 2H), 1.54-1.39 (m, 2H).

### Example 306: 2-((1-(4-(2-(2-Aminopyridin-3-yl)-5-(2-chlorophenyl)-3H-imidazo[4,5-b]pyridin-3-yl)benzyl)piperidin-4-yl)amino)pyrimidine-4-carbonitrile

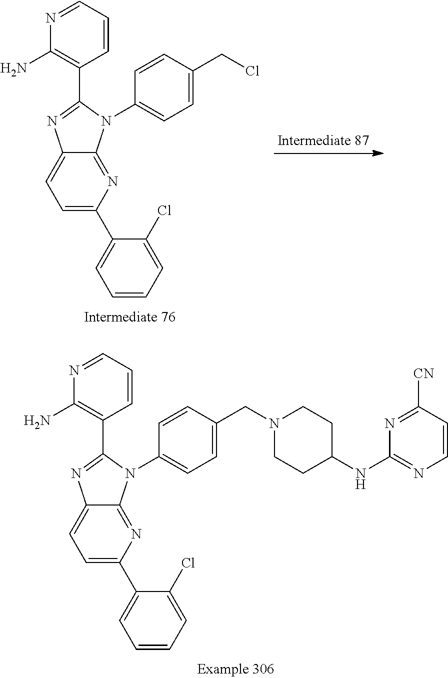 [SMILES: Nc1ncccc1-c1nc2ccc(-c3ccccc3Cl)nc2n1-c1ccc(CCl)cc1 ; N#Cc1ccnc(NC2CCN(Cc3ccc(-n4c(-c5cccnc5N)nc5ccc(-c6ccccc6Cl)nc54)cc3)CC2)n1]

To a solution of Intermediate 76 (200 mg, 414 μmol) and Intermediate 87 (131 mg, 414 μmol) in DMF (3 mL) were added NaI (31.1 mg, 207 μmol) and K₂CO₃ (286 mg, 2.07 mmol). The mixture was stirred at 50° C. for 0.5 hr. The reaction mixture was filtered. The residue was purified by prep-HPLC (column: Waters xbridge 150*25 mm*10 μm; mobile phase: [water (NH₄HCO₃)-ACN]; gradient: B: 50%-70% 14 min) to give 2-((1-(4-(2-(2-aminopyridin-3-yl)-5-(2-chlorophenyl)-3H-imidazo[4,5-h]pyridin-3-yl)benzyl)piperidin-4-yl)amino)pyrimidine-4-carbonitrile (Example 306, 45 mg, yield: 17%) as a light-yellow solid. MS: m/z=613.3 [M+H]⁺. ¹H NMR (400 MHz, Dimethylsulfoxide-d₆) δ 8.61 (d, J=4.8 Hz, 1H), 8.29 (d, J=8 Hz, 1H), 8.00 (d, J=4.8 Hz, 1H), 7.63 (d, J=8.4 Hz, 1H), 7.57-7.37 (m, 9H), 7.21 (d, J=7.6 Hz, 1H), 7.09 (d, J=4.4 Hz, 1H), 6.96 (br s, 2H), 6.40 (dd, J=7.6, 4.8 Hz, 1H), 4.42-4.33 (m, 2H), 3.82 (s, 2H), 3.37-3.35 (m, 2H), 3.17-3.09 (m, 2H), 2.78-2.70 (m, 1H₁), 1.93-1.86 (m, 2H₁).

### Example 307: 6-((1-(4-(2-(2-Aminopyridin-3-yl)-5-(2-chlorophenyl)-3H-imidazo[4,5-b]pyridin-3-yl)benzyl)piperidin-4-yl)amino)pyrimidine-4-carbonitrile

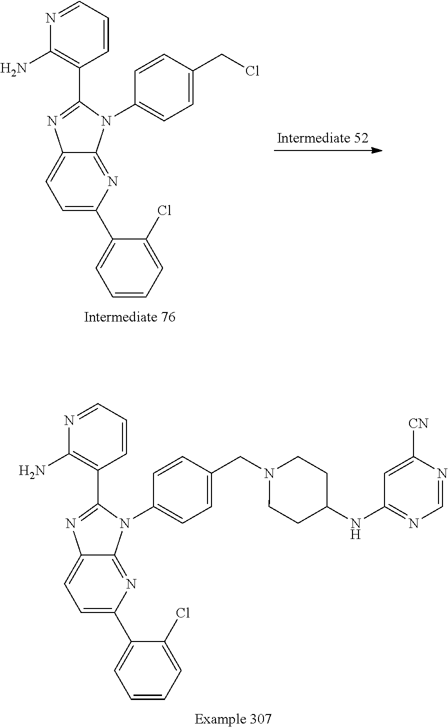 [SMILES: Nc1ncccc1-c1nc2ccc(-c3ccccc3Cl)nc2n1-c1ccc(CCl)cc1 ; N#Cc1cc(NC2CCN(Cc3ccc(-n4c(-c5cccnc5N)nc5ccc(-c6ccccc6Cl)nc54)cc3)CC2)ncn1]

To a solution of Intermediate 76 (200 mg, 414 μmol, HCl) and Intermediate 52 (131 mg, 414 μmol, TFA) in DMF (3 mL) were added NaI (31.1 mg, 207 μmol) and K₂CO₃ (286 mg, 2.07 mmol). The mixture was stirred at 50° C. for 1 hr. After cooling to 25° C., the reaction mixture was diluted with H₂O (20 mL) and extracted with EtOAc (15 mL×2). The combined organic layers were washed with brine (30 mL), dried over anhydrous Na₂SO₄, filtered and concentrated under reduced pressure. After purified by prep-HPLC (column: Waters xbridge 150×25 mm 10 μm; mobile phase: [water (NH₄HCO₃)-ACN]; gradient: 45%-75% B over 14 min), 6-((1-(4-(2-(2-aminopyridin-3-yl)-5-(2-chlorophenyl)-3H-imidazo[4,5-b]pyridin-3-yl)benzyl)piperidin-4-yl)amino)pyrimidine-4-carbonitrile (Example 307, 43.3 mg, yield: 17%) was obtained as a yellow solid. MS: m/z=613.4 [M+H]⁺. ¹H NMR (400 MHz, Dimethylsulfoxide-d₆) δ 8.49 (s, 1H), 8.29 (d, J=8.4 Hz, 1H), 8.06 (d, J=6.8 Hz, 1H), 8.00 (dd, J=5.2, 2.0 Hz, 1H), 7.63 (d, J=8.4 Hz, 1H), 7.58-7.53 (m, 2H), 7.45-7.42 (m, 6H), 7.16 (dd, J=7.6, 2.0 Hz, 1H), 6.97 (br s, 2H), 6.92 (s, 1H), 6.38 (dd, J=7.6, 4.8 Hz, 1H), 3.93-3.75 (m, 1H), 3.54 (s, 2H) 2.85-2.75 (m, 2H), 2.16-2.07 (m, 2H), 1.93-1.84 (m, 2H), 1.52-1.43 (m, 2H).

### Example 308: 4-((1-(4-(2-(2-Aminopyridin-3-yl)-5-(4-chlorophenyl)-3H-imidazo[4,5-b]pyridin-3-yl)benzyl)piperidin-4-yl)amino)pyrimidine-2-carbonitrile

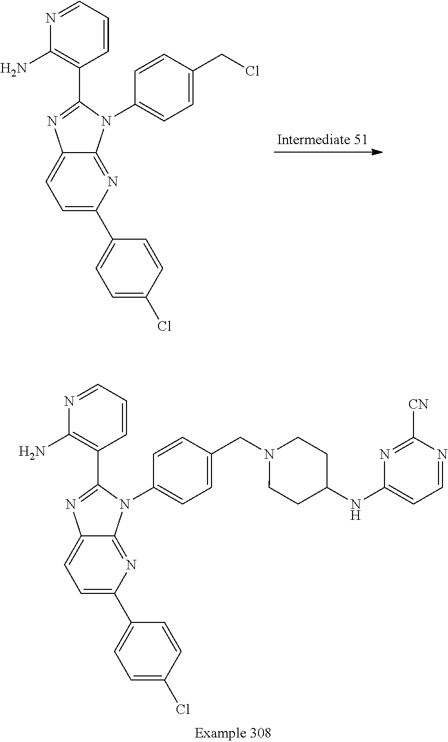 [SMILES: Nc1ncccc1-c1nc2ccc(-c3ccc(Cl)cc3)nc2n1-c1ccc(CCl)cc1 ; N#Cc1nccc(NC2CCN(Cc3ccc(-n4c(-c5cccnc5N)nc5ccc(-c6ccc(Cl)cc6)nc54)cc3)CC2)n1]

To a solution of 3-(3-(4-(chloromethyl)phenyl)-5-(4-chlorophenyl)-3H-imidazo[4,5-b]pyridin-2-yl)pyridin-2-amine (refer to Intermediate 43 for detail procedures, 250 mg, 518 μmol) and Intermediate 51 (164 mg, 518 μmol) in DMF (2 mL) were added NaI (15.5 mg, 104 μmol) and K₂CO₃ (358 mg, 2.59 mmol). The mixture was stirred at 50° C. for 1 hr. The reaction mixture was diluted with water (10 mL) and extracted with EtOAc (10 mL×2). The combined organic layers were washed with brine (10 mL), dried over Na₂SO₄, filtered and concentrated under reduced pressure. The residue was purified by silica gel flash chromatography (Eluent of 0˜7% MeOH in CH₂Cl₂) to give 4-((1-(4-(2-(2-aminopyridin-3-yl)-5-(4-chlorophenyl)-3H-imidazo[4,5-b]pyridin-3-yl)benzyl)piperidin-4-yl)amino)pyrimidine-2-carbonitrile (Example 308, 40.6 mg, yield: 12%) as a yellow solid. MS: m/z=613.2 [M+H]⁺. ¹H NMR (400 MHz, Dimethylsulfoxide-d₆) δ 8.28 (d, J=8.4 Hz, 1H), 8.11-7.98 (m, 6H), 7.54-7.44 (m, 6H), 7.15 (dd, J=7.6, 1.6 Hz, 1H), 7.03 (br s, 2H), 6.68 (d, J=6.0 Hz, 1H), 6.37 (dd, J=7.6, 4.8 Hz, 1H), 3.88-3.75 (m, 1H), 3.59 (s, 2H), 2.87-2.79 (d, J=11.2 Hz, 2H), 2.16 (m, 2H), 1.90 (d, J=11.2 Hz, 2H), 1.56-1.43 (m, 2H).

### Example 309: 2-(4-(4-(2-(2-Aminopyridin-3-yl)-5-(3-fluorophenyl)-3H-imidazo[4,5-b]pyridin-3-yl)benzyl)piperazin-1-yl)pyrimidine-4-carbonitrile

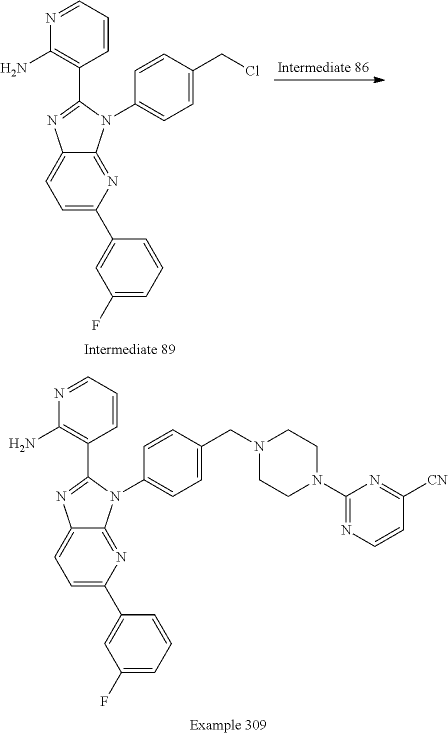 [SMILES: Nc1ncccc1-c1nc2ccc(-c3cccc(F)c3)nc2n1-c1ccc(CCl)cc1 ; N#Cc1ccnc(N2CCN(Cc3ccc(-n4c(-c5cccnc5N)nc5ccc(-c6cccc(F)c6)nc54)cc3)CC2)n1]

To a solution of Intermediate 89 (200 mg, 429 μmol) and Intermediate 86 (130 mg, 429 μmol) in DMF (2 mL) were added NaI (32.1 mg, 214 μmol) and K₂CO₃ (296 mg, 2.14 mmol). The mixture was stirred at 50° C. for 0.5 hr. The reaction mixture was filtered. The residue was purified by prep-HPLC (column: Waters xbridge 150*25 mm*10 μm; mobile phase: [water (NH₄HCO₃)-ACN]; gradient: 54%-74% B over 14 min) to give 2-(4-(4-(2-(2-aminopyridin-3-yl)-5-(3-fluorophenyl)-3H-imidazo[4,5-b]pyridin-3-yl)benzyl)piperazin-1-yl)pyrimidine-4-carbonitrile (Example 309, 51.5 mg, yield: 20%) as a light-yellow solid. MS: m/z=583.4 [M+H]⁺. ¹H NMR (400 MHz, Dimethylsulfoxide-d₆) δ 8.65 (d, J=4.4 Hz, 1H), 8.29 (d, J=8.4 Hz, 1H), 8.05 (d, J=8.4 Hz, 1H), 8.02-7.99 (m, 1H), 7.91 (d, J=8.0 Hz, 1H), 7.83 (d, J=10.0 Hz, 1H), 7.54-7.46 (m, 5H), 7.27-7.21 (m, 1H), 7.19-7.14 (m, 2H), 7.04 (br s, 2H), 6.38 (dd, 1=8.0, 5.2 Hz, 1H), 3.83-3.75 (m, 4H), 3.65 (s, 2H), 3.38-3.33 (m, 4H). ¹⁹F NMR (400 MHz, Dimethylsulfoxide-d₆) δ −112.91.

### Example 310: 6-(4-(4-(2-(2-Aminopyridin-3-yl)-5-(3-fluorophenyl)-3H-imidazo[4,5-b]pyridin-3-yl)benzyl)piperazin-1-yl)pyrimidine-4-carbonitrile

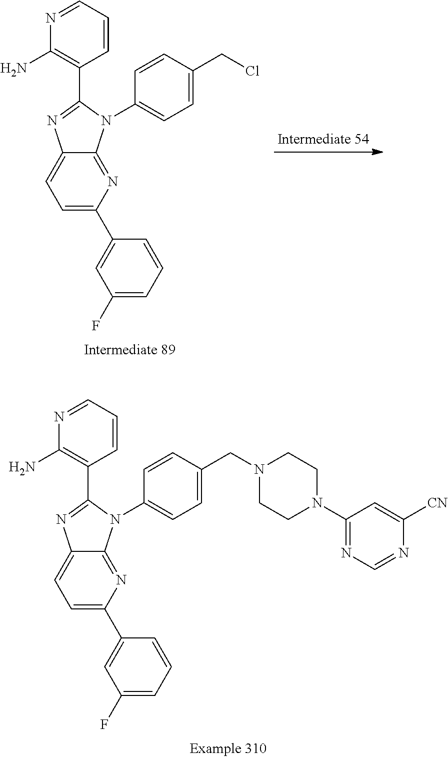 [SMILES: Nc1ncccc1-c1nc2ccc(-c3cccc(F)c3)nc2n1-c1ccc(CCl)cc1 ; N#Cc1cc(N2CCN(Cc3ccc(-n4c(-c5cccnc5N)nc5ccc(-c6cccc(F)c6)nc54)cc3)CC2)ncn1]

To a solution of Intermediate 89 (200 mg, 429 μmol) and Intermediate 54 (130 mg, 429 μmol) in DMF (3 mL) were added NaI (32.1 mg, 214 μmol) and K₂CO₃ (296 mg, 2.14 mmol). The mixture was stirred at 50° C. for 0.5 hr. The reaction mixture was filtered. The residue was purified by prep-HPLC (column: Waters xbridge 150*25 mm*10 μm; mobile phase: [water (NH₄HCO₃)-ACN]; gradient: 46%-66% B over 14 min) to give 6-(4-(4-(2-(2-aminopyridin-3-yl)-5-(3-fluorophenyl)-3H-imidazo[4,5-b]pyridin-3-yl)benzyl)piperazin-1-yl)pyrimidine-4-carbonitrile (Example 310, 24.6 mg, yield: 9.6%) as a light-yellow solid. MS: m/z=583.4 [M+H]⁺. ¹H NMR (400 MHz, Dimethylsulfoxide-d₆) δ 8.57 (s, 1H), 8.29 (d, J=8.4 Hz, 1H), 8.05 (d, J=8.4 Hz, 1H), 8.00 (dd, J=4.8, 1.6 Hz, 1H), 7.91 (d, J=7.6 Hz, 1H), 7.83 (d, J=10.4 Hz, 1H), 7.58-7.56 (m, 1H), 7.54-7.47 (m, 5H), 7.27-7.21 (m, 1H), 7.16 (dd, J=7.6, 1.6 Hz, 1H), 7.04 (br s, 2H), 6.36 (dd, J=7.6, 4.8 Hz, 1H), 3.83-3.69 (m, 4H), 3.65 (s, 2H), 3.38-3.34 (m, 2H), 3.30-3.26 (m, 2H). ¹⁹F NMR (377 MHz, Dimethylsulfoxide-d₆) δ −112.89.

### Example 311: 6-((1-(4-(2-(2-Aminopyridin-3-yl)-5-(3-fluorophenyl)-3H-imidazo[4,5-b]pyridin-3-yl)benzyl)piperidin-4-yl)amino)pyrimidine-4-carbonitrile

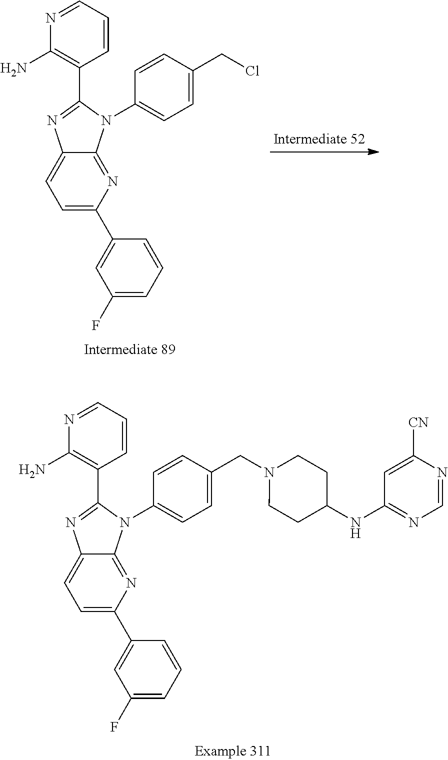 [SMILES: Nc1ncccc1-c1nc2ccc(-c3cccc(F)c3)nc2n1-c1ccc(CCl)cc1 ; N#Cc1cc(NC2CCN(Cc3ccc(-n4c(-c5cccnc5N)nc5ccc(-c6cccc(F)c6)nc54)cc3)CC2)ncn1]

To a solution of Intermediate 89 (200 mg, HCl salt, 429 μmol) and Intermediate 52 (87.2 mg, TFA salt, 275 μmol) in DMF (3 mL) were added NaI (32.1 mg, 214 μmol) and K₂CO₃ (296 mg, 2.14 mmol). The mixture was stirred at 50° C. for 1 hr. After cooling to room temperature, the reaction mixture was diluted with H₂O (20 mL) and extracted with EtOAc (15 mL×2). The combined organic layers were washed with brine (30 mL), dried over anhydrous Na₂SO₄, filtered and concentrated under reduced pressure. After purified by prep-HPLC (column: Waters xbridge 150×25 mm 10 μm; mobile phase: [water (NH₄HCO₃)-ACN]; gradient: 45%-75% B over 14 min), 6-((1-(4-(2-(2-aminopyridin-3-yl)-5-(3-fluorophenyl)-3H-imidazo[4,5-b]pyridin-3-yl)benzyl)piperidin-4-yl)amino)pyrimidine-4-carbonitrile (Example 311, 9.6 mg, yield: 3.5%) was obtained as a yellow solid. MS: m/z=597.4 [M+H]⁺. ¹H NMR (400 MHz, Dimethylsulfoxide-d₆) δ 8.51-8.42 (m, 1H), 8.29 (d, J=8.4 Hz, 1H), 8.10-8.04 (m, 2H), 8.00 (dd, J=4.8, 2.0 Hz, 1H), 7.91 (d, J=10.8 Hz, 1H), 7.84-7.81 (m, 1H), 7.52-7.45 (m, 5H), 7.25-7.16 (m, 2H), 7.04 (br s, 2H), 6.93 (s, 1H), 6.38 (dd, J=7.6, 4.8 Hz, 1H), 3.92-3.81 (m, 1H), 3.59 (s, 2H), 2.86-2.81 (m, 2H), 2.16-2.10 (m, 2H), 1.94-1.86 (m, 2H), 1.55-1.46 (m, 2H). ¹⁹F NMR (400 MHz, Dimethylsulfoxide-d₆) δ −112.913.

### Example 312: 4-(4-(4-(2-(2-Aminopyridin-3-yl)-5-(2-fluorophenyl)-3H-imidazo[4,5-b]pyridin-3-yl)benzyl)piperazin-1-yl)-1,3,5-triazine-2-carbonitrile

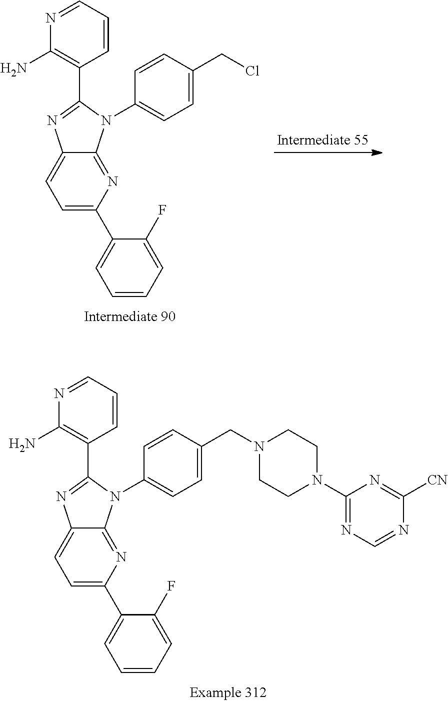 [SMILES: Nc1ncccc1-c1nc2ccc(-c3ccccc3F)nc2n1-c1ccc(CCl)cc1 ; N#Cc1ncnc(N2CCN(Cc3ccc(-n4c(-c5cccnc5N)nc5ccc(-c6ccccc6F)nc54)cc3)CC2)n1]

To a solution of Intermediate 90 (300 mg, 643 μmol) and Intermediate 55 (196 mg, 643 mol) in DMF (2 mL) were added NaI (48.2 mg, 322 μmol) and K₂CO₃ (445 mg, 3.22 mmol). The mixture was stirred at 50° C. for 2 hr. The reaction mixture was filtered and the residue was purified by prep-HPLC (column: [water (NH₄HCO₃)-ACN]; B %: 47%-77%, 14 min) to give 4-(4-(4-(2-(2-aminopyridin-3-yl)-5-(2-fluorophenyl)-3H-imidazo[4,5-b]pyridin-3-yl)benzyl)piperazin-1-yl)-1,3,5-triazine-2-carbonitrile (Example 312, 103 mg, yield: 26%) as a yellow solid. MS: m/z=584.2 [M+H]⁺. ¹H NMR (400 MHz, Dimethylsulfoxide-d₆) δ 8.72 (s, 1H), 8.30 (d, J=8.4 Hz, 1H), 8.00 (dd, J=4.8, 2.0 Hz, 1H), 7.83-7.77 (m, 2H), 7.51-7.44 (m, 5H), 7.36-7.29 (m, 2H), 7.17 (dd, J=7.6, 2.0 Hz, 1H), 6.98 (br s, 2H), 6.39 (dd, J=7.6, 4.8 Hz, 1H), 3.87-3.79 (m, 4H), 3.63 (s, 2H), 2.54-2.51 (m, 4H). ¹⁹F NMR (400 MHz, Dimethylsulfoxide-d₆) δ −117.168.

### Example 313: 2-((1-(4-(2-(2-Aminopyridin-3-yl)-5-(2-fluorophenyl)-3H-imidazo[4,5-b]pyridin-3-yl)benzyl)piperidin-4-yl)amino)pyrimidine-4-carbonitrile

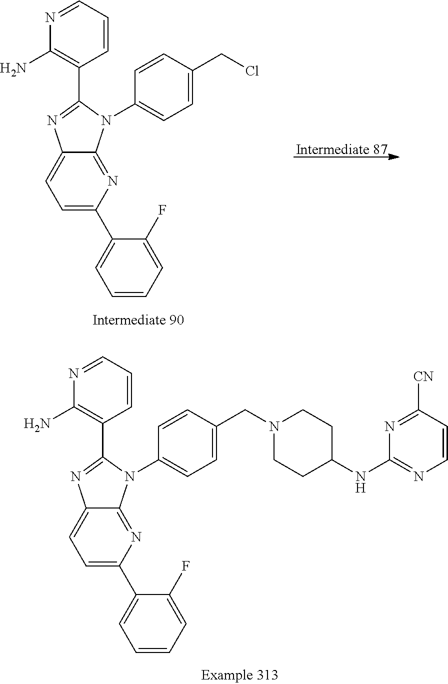 [SMILES: Nc1ncccc1-c1nc2ccc(-c3ccccc3F)nc2n1-c1ccc(CCl)cc1 ; N#Cc1ccnc(NC2CCN(Cc3ccc(-n4c(-c5cccnc5N)nc5ccc(-c6ccccc6F)nc54)cc3)CC2)n1]

To a solution of Intermediate 90 (193 mg, 449 μmol) and Intermediate 87 (143 mg, 702 μmol) in DMF (3 mL) were added NaI (33.7 mg, 225 μmol) and K₂CO₃ (311 mg, 2.25 mmol). The mixture was stirred at 50° C. for 0.5 hr. The reaction mixture was filtered. The residue was purified by prep-HPLC (column: Phenomenex C18 150*25 mm*10 μm; mobile phase: [water (NH₄HCO₃)-ACN]; gradient: 49%-79% B over 10 min) to give 2-((1-(4-(2-(2-aminopyridin-3-yl)-5-(2-fluorophenyl)-3H-imidazo[4,5-b]pyridin-3-yl)benzyl)piperidin-4-yl)amino)pyrimidine-4-carbonitrile (Example 313, 18.5 mg, yield: 6.8%) as a light-yellow solid. MS: m/z=597.4 [M+H]⁺. 1H NMR (400 MHz, Dimethylsulfoxide-d₆) δ 8.61 (d, J=4.4 Hz, 1H), 8.29 (d, J=8.4 Hz, 1H), 8.00 (dd, J=4.8, 1.6 Hz, 1H), 7.83-7.77 (m, 2H), 7.52-7.39 (m, 6H), 7.35-7.29 (m, 2H), 7.20 (dd, J=7.6, 0.8 Hz, 1H), 7.10 (d, J=4.8 Hz, 1H), 6.98 (br s, 2H), 6.40 (dd, J=7.6, 5.2 Hz, 1H), 4.41-4.37 (m, 1H), 3.83 (s, 2H), 3.14 (t, J=11.2 Hz 2H), 2.81-2.70 (m, 2H), 1.93-1.87 (m, 2H), 1.30-1.23 (m, 2H). ¹⁹F NMR (377 MHz, Dimethylsulfoxide-d₆) δ −117.15.

### Example 314: 6-(4-(4-(2-(2-Aminopyridin-3-yl)-5-(2-fluorophenyl)-3H-imidazo[4,5-b]pyridin-3-yl)benzyl)piperazin-1-yl)pyrimidine-4-carbonitrile

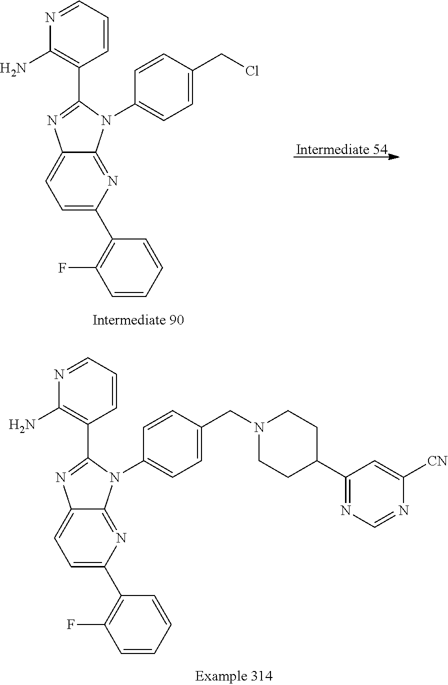 [SMILES: Nc1ncccc1-c1nc2ccc(-c3ccccc3F)nc2n1-c1ccc(CCl)cc1 ; N#Cc1cc(C2CCN(Cc3ccc(-n4c(-c5cccnc5N)nc5ccc(-c6ccccc6F)nc54)cc3)CC2)ncn1]

To a solution of Intermediate 90 (200 mg, 429 μmol) and Intermediate 54 (130 mg, 429 μmol) in DMF (3 mL) were added NaI (32.1 mg, 214 μmol) and K₂CO₃ (296 mg, 2.14 mmol). The mixture was stirred at 50° C. for 0.5 hr. The reaction mixture was filtered. The residue was purified by prep-HPLC (column: Waters xbridge 150*25 mm*10 μm; mobile phase: [water (NH₄HCO₃)-ACN]; gradient: 47%-77% B over 14 min) to give 6-(4-(4-(2-(2-Aminopyridin-3-yl)-5-(2-fluorophenyl)-3H-imidazo[4,5-b]pyridin-3-yl)benzyl)piperazin-1-yl)pyrimidine-4-carbonitrile (Example 314, 46.5 mg, yield: 17%) as a light-yellow solid. MS: m/z=583.4 [M+H]⁺. ¹H NMR (400 MHz, Dimethylsulfoxide-d₆) δ 8.56 (s, 1H), 8.30 (d, J=8.4 Hz, 1H), 8.00 (d, J=4.4 Hz, 1H), 7.83-7.78 (m, 2H), 7.56 (s, 1H), 7.50-7.45 (m, 5H), 7.36-7.28 (m, 2H), 7.16 (d, J=7.6 Hz, 1H), 7.00 (br s, 2H), 6.36 (dd, J=7.6, 4.8 Hz, 1H), 3.78-3.70 (m, 4H), 3.62 (s, 2H), 2.48-2.46 (m, 4H). ¹⁹F NMR (377 MHz, Dimethylsulfoxide-d₆) δ −117.16.

### Example 315: 6-((1-(4-(2-(2-Aminopyridin-3-yl)-5-(2-fluorophenyl)-3H-imidazo[4,5-b]pyridin-3-yl)benzyl)piperidin-4-yl)amino)pyrimidine-4-carbonitrile

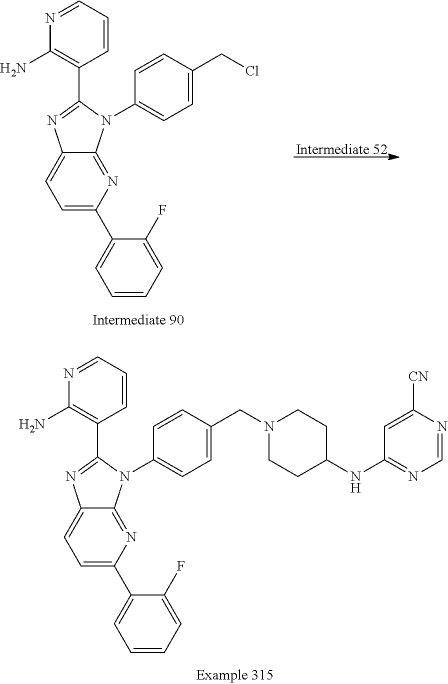 [SMILES: Nc1ncccc1-c1nc2ccc(-c3ccccc3F)nc2n1-c1ccc(CCl)cc1 ; N#Cc1cc(NC2CCN(Cc3ccc(-n4c(-c5cccnc5N)nc5ccc(-c6ccccc6F)nc54)cc3)CC2)ncn1]

To a solution of Intermediate 90 (200 mg HCl salt, 429 μmol) and Intermediate 52 (136 mg, 429 μmol, TFA) in DMF (3 mL) were added NaI (32.1 mg, 214 μmol) and K₂CO₃ (296 mg, 2.14 mmol). The mixture was stirred at 50° C. for 1 hr. After cooling to 25° C., the reaction mixture was diluted with H₂O (20 mL) and extracted with EtOAc (15 mL×2). The combined organic layers were washed with brine (30 mL), dried over anhydrous Na₂SO₄, filtered and concentrated under reduced pressure. After purified by prep-HPLC (column: Waters xbridge 150×25 mm 10 μm; mobile phase. [water (NH₄HCO₃)-ACN]; gradient: 44%-74% B over 14 min), 6-((1-(4-(2-(2-aminopyridin-3-yl)-5-(2-fluorophenyl)-3H-imidazo[4,5-b]pyridin-3-yl)benzyl)piperidin-4-yl)amino)pyrimidine-4-carbonitrile (Example 315, 15 mg, 5.6% yield) was obtained as a yellow solid. MS: m/z=597.4 [M+H]⁺. ¹H NMR (400 MHz, Dimethylsulfoxide-d₆) δ 8.52-8.42 (m, 1H), 8.29 (d, J=12.0 Hz, 1H), 8.07 (d, J=7.6 Hz, 1H), 8.00 (dd, J=4.8, 2.0 Hz, 1H), 7.83-7.78 (m, 2H), 7.48-7.43 (m, 5H), 7.35-7.29 (m, 2H), 7.16 (dd, J=7.6, 1.6 Hz, 1H), 6.99 (br s, 2H), 6.92 (s, 1H), 6.38 (dd, J=7.6, 4.8 Hz, 1H), 3.93-3.80 (m, 1H), 3.56 (s, 2H), 2.83-2.78 (m, 2H), 2.15-2.08 (m, 2H), 1.93-1.83 (m, 2H), 1.54-1.44 (m, 2H). ¹⁹F NMR (400 MHz, Dimethylsulfoxide-d₆) δ −117.177.

### Example 316: 4-((1-(4-(2-(2-Aminopyridin-3-yl)-5-(2-fluorophenyl)-3H-imidazo[4,5-b]pyridin-3-yl)benzyl)piperidin-4-yl)amino)pyrimidine-2-carbonitrile

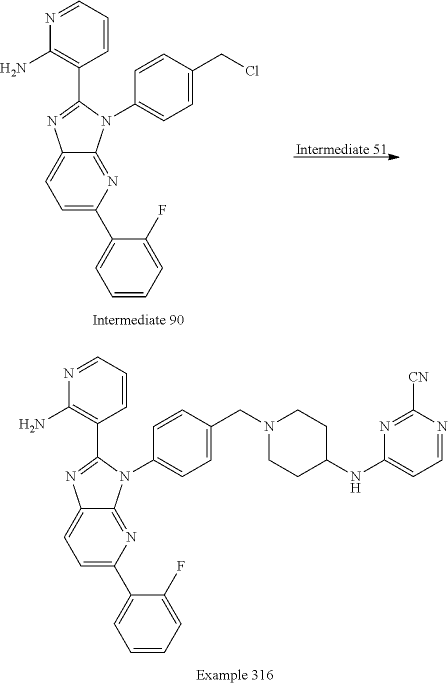 [SMILES: Nc1ncccc1-c1nc2ccc(-c3ccccc3F)nc2n1-c1ccc(CCl)cc1 ; N#Cc1nccc(NC2CCN(Cc3ccc(-n4c(-c5cccnc5N)nc5ccc(-c6ccccc6F)nc54)cc3)CC2)n1]

To a solution of Intermediate 90 (250 mg HCl salt, 517 μmol) and Intermediate 51 (164 mg TFA salt, 517 μmol) in DMF (2 mL) were added NaI (15.5 mg, 103 μmol) and K₂CO₃ (357 mg, 2.59 mmol). The mixture was stirred at 50° C. for 1 hr. The reaction mixture was diluted with water (10 mL) and extracted with EtOAc (10 mL×2). The combined organic layers were washed with brine (10 mL), dried over Na₂SO₄, filtered and concentrated under reduced pressure. The residue was purified by silica gel flash chromatography (Eluent of 0˜7% MeOH in CH₂Cl₂) to give 4-((1-(4-(2-(2-aminopyridin-3-yl)-5-(2-fluorophenyl)-3H-imidazo[4,5-b]pyridin-3-yl)benzyl)piperidin-4-yl)amino)pyrimidine-2-carbonitrile (Example 316, 55.3 mg, yield: 17%,) as a yellow solid. MS: m/z=597.4 [M+H]⁺. ¹H NMR (400 MHz, Dimethylsulfoxide-d₆) δ 8.30 (d, J=8.0 Hz, 1H), 8.13-7.96 (m, 3H), 7.84-7.75 (m, 2H), 7.50-7.42 (m, 5H), 7.36-7.28 (m, 2H), 7.16 (dd, J=7.6, 1.6 Hz, 1H), 7.00 (br s, 2H), 6.67 (d, J=6.0 Hz, 1H), 6.38 (dd, J=8.0, 4.8 Hz, 1H), 3.87-3.74 (m, 1H), 3.57 (s, 2H), 2.85-2.76 (m, 2H), 2.14 (m, 2H), 1.88 (d, J=10.0 Hz, 2H), 1.54-1.41 (m, 2H). ¹⁹F NMR (400 MHz, Dimethylsulfoxide-d₆) δ −117.176.

### Example 317: 4-(4-(4-(2-(2-Aminopyridin-3-yl)-5-(3-oxo-1,4-diazepan-1-yl)-3H-imidazo[4,5-b]pyridin-3-yl)benzyl)piperazin-1-yl)pyrimidine-2-carbonitrile

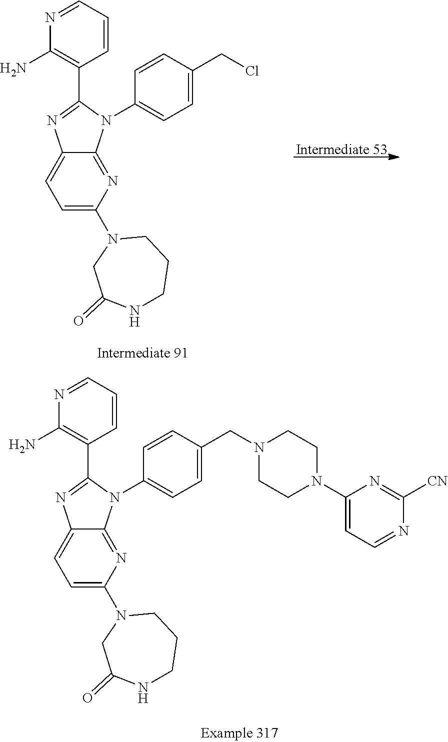 [SMILES: Nc1ncccc1-c1nc2ccc(N3CCCNC(=O)C3)nc2n1-c1ccc(CCl)cc1 ; N#Cc1nccc(N2CCN(Cc3ccc(-n4c(-c5cccnc5N)nc5ccc(N6CCCNC(=O)C6)nc54)cc3)CC2)n1]

To a solution of Intermediate 91 (150 mg, 335 μmol) and Intermediate 53 (117 mg, 368 μmol) in DMF (5 mL) were added NaI (10.0 mg, 66.9 μmol) and K₂CO₃ (139 mg, 1.00 mmol) in one portion at 20° C. The mixture was stirred at 20° C. for 12 hr and 80° C. for 2 hr. Water (10 mL) was added at 20° C. and extracted with EtOAc (20 mL×3). The combined organic layers were washed with water (20 mL×2), dried over anhydrous Na₂SO₄, filtered and concentrated under reduced pressure. The residue was purified by prep-HPLC (column: Phenomenex C18 75×30 mm×3 μm; mobile phase: [water (NH₃H₂O+NH₄HCO₃)-ACN]; gradient: 28%-58% B over 7 min) to give 4-(4-(4-(2-(2-aminopyridin-3-yl)-5-(3-oxo-1,4-diazepan-1-yl)-3H-imidazo[4,5-b]pyridin-3-yl)benzyl)piperazin-1-yl)pyrimidine-2-carbonitrile (Example 317, 40.8 mg, yield: 20%) as a yellow solid. MS: m/z=601.2 [M+H]⁺. ¹H NMR (400 MHz, Dimethylsulfoxide-d₆) δ 8.27 (d, J=6.4 Hz, 1H), 7.97-7.92 (m, 2H), 7.47-7.42 (m, 3H), 7.35 (d, J=8.0 Hz, 2H), 7.12 (d, J=6.4 Hz, 1H), 7.02-6.79 (m, 3H), 6.80 (d, J=8.8 Hz, 1H), 6.32 (dd, J=7.6, 5.2 Hz 1H), 4.18 (s, 2H), 3.82-3.68 (m, 6H), 3.62 (s, 2H), 3.25-3.05 (m, 2H), 2.49-2.42 (m, 4H), 1.65-1.62 (m, 2H).

### Example 318: 4-((1-(4-(2-(2-Aminopyridin-3-yl)-5-(3-oxo-1,4-diazepan-1-yl)-3H-imidazo[4,5-b]pyridin-3-yl)benzyl)piperidin-4-yl)amino)pyrimidine-2-carbonitrile

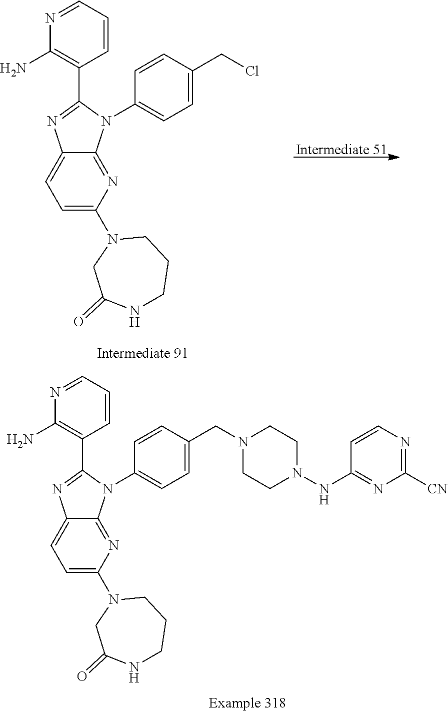 [SMILES: Nc1ncccc1-c1nc2ccc(N3CCCNC(=O)C3)nc2n1-c1ccc(CCl)cc1 ; N#Cc1nccc(NN2CCN(Cc3ccc(-n4c(-c5cccnc5N)nc5ccc(N6CCCNC(=O)C6)nc54)cc3)CC2)n1]

To a solution of Intermediate 91 (200 mg, 447 μmol) and Intermediate 51 (99.8 mg, 491 μmol) in DMF (5 mL) were added K₂CO₃ (185 mg, 1.34 mmol) and NaI (13.4 mg, 89.3 μmol) in one portion at 20° C. The mixture was stirred at 20° C. for 12 hr. The reaction mixture was stirred at 80° C. for 2 hr. Water (10 mL) was added at 20° C. and extracted with EtOAc (20 mL×3). The combined organic layers were washed with water (20 mL×2), dried over anhydrous Na₂SO₄, filtered and concentrated under reduced pressure. The residue was purified by prep-HPLC (column: Boston Prime C18 150×30 mm×5 μm; mobile phase: [water (NH₃H₂O+NH₄HCO₃)-ACN]; gradient: 37%-67% B over 7 min) to give 4-((1-(4-(2-(2-aminopyridin-3-yl)-5-(3-oxo-1,4-diazepan-1-yl)-3H-imidazo[4,5-b]pyridin-3-yl)benzyl)piperidin-4-yl)amino)pyrimidine-2-carbonitrile (Example 318, 33.1 mg, yield: 12%) as an off-white solid. MS: m/z=615.2 [M+H]⁺. ¹H NMR (400 MHz, Dimethylsulfoxide-d₆) δ 8.09-7.95 (m, 2H), 7.94-7.92 (m, 2H), 7.46-7.43 (m, 1H), 7.41 (d, J=8.0 Hz, 2H), 7.33 (d, J=8.4 Hz, 2H), 7.02-6.99 (m, 3H), 6.80 (d, J=9.2 Hz, 1H), 6.65 (d, J=8.0 Hz, 1H), 6.34-6.30 (m, 1H), 4.17 (s, 2H), 3.83-3.75 (m, 3H), 3.56 (s, 2H), 3.19-3.15 (m, 2H), 2.82-2.78 (m, 2H), 2.16-2.09 (m, 2H), 1.89-1.86 (m, 2H), 1.68-1.62 (m, 2H), 1.49-1.45 (m, 2H).

### Example 319: 2-(4-(4-(2-(2-Aminopyridin-3-yl)-5-(4-cyanophenyl)-3H-imidazo[4,5-b]pyridin-3-yl)benzyl)piperazin-1-yl)pyrimidine-4-carbonitrile

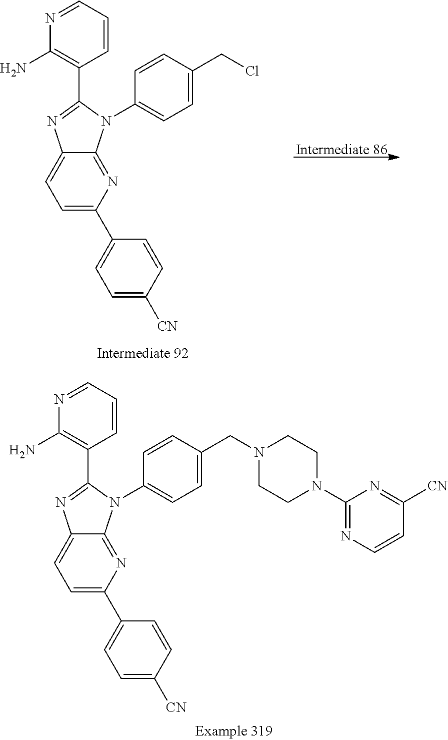 [SMILES: N#Cc1ccc(-c2ccc3nc(-c4cccnc4N)n(-c4ccc(CCl)cc4)c3n2)cc1 ; N#Cc1ccc(-c2ccc3nc(-c4cccnc4N)n(-c4ccc(CN5CCN(c6nccc(C#N)n6)CC5)cc4)c3n2)cc1]

To a solution of Intermediate 92 (100 mg, 229 μmol) and Intermediate 86 (43 mg, 229 μmol) in DMF (2 mL) were added K₂CO₃ (94.9 mg, 687 μmol) and NaI (6.86 mg, 45.8 μmol). The mixture was stirred at 50° C. for 0.5 hr. The reaction mixture was filtered to give a filter liquid. After purified byprep-HPLC (column: Phenomenex C₁₈ 150×25 mm×10 μm; mobile phase: [water (NH₄HCO₃)-ACN]: B: 48%-68%, 14 min), 2-(4-(4-(2-(2-aminopyridin-3-yl)-5-(4-cyanophenyl)-3H-imidazo[4,5-b]pyridin-3-yl)benzyl)piperazin-1-yl)pyrimidine-4-carbonitrile (Example 319, 23.2 mg, yield: 17%) was obtained as a yellow solid. MS: m/z=590.2 [M+H]⁺. ¹H NMR (400 MHz, Dimethylsulfoxide-d₆) δ 8.65 (d, J=4.8 Hz, 1H), 8.34 (d, J=8.4 Hz, 1H), 8.24 (d, J=8.4 Hz, 2H), 8.13 (d, J=8.4 Hz, 1H), 8.01 (dd, J=4.8, 2.0 Hz, 1H), 7.94 (d, J=8.4 Hz, 2H), 7.54-7.46 (m, 4H), 7.19-7.14 (m, 2H), 7.03 (br s, 2H), 6.38 (dd, J=7.6, 4.8 Hz, 1H), 3.84-3.78 (m, 4H), 3.64 (s, 2H), 3.30-3.29 (m, 4H).

### Example 320: 4-((1-(4-(2-(2-Aminopyridin-3-yl)-5-(4-cyanophenyl)-3H-imidazo[4,5-b]pyridin-3-yl)benzyl)piperidin-4-yl)amino)pyrimidine-2-carbonitrile

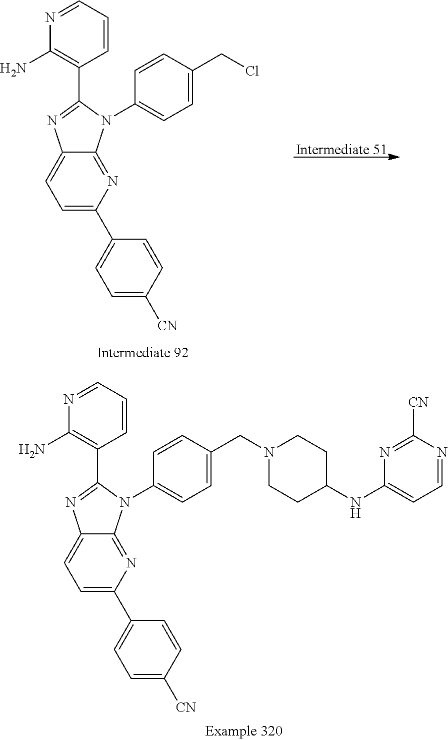 [SMILES: N#Cc1ccc(-c2ccc3nc(-c4cccnc4N)n(-c4ccc(CCl)cc4)c3n2)cc1 ; N#Cc1ccc(-c2ccc3nc(-c4cccnc4N)n(-c4ccc(CN5CCC(Nc6ccnc(C#N)n6)CC5)cc4)c3n2)cc1]

To a solution of Intermediate 92 (100 mg, 229 μmol) and Intermediate 51 (46.5 mg, 229 μmol) in DMF (2 mL) were added K₂CO₃ (94.9 mg, 687 μmol) and NaI (6.86 mg, 45.8 μmol). The mixture was stirred at 50° C. for 0.5 hr. The reaction mixture was filtered. After purified by prep-HPLC (column: Phenomenex Luna C₁₈ 150×25 mm×10 μm; mobile phase: [water (HCl)-ACN]; gradient: 15%-45% B over 14 min), the preparation solution was diluted with H₂O (10 mL) and adjust pH=7 with NaHCO₃, and then extracted with CH₂Cl₂ (5 mL×2). The combined organic layers were washed with aqueous brine (15 mL), dried over Na₂SO₄, filtered and concentrated under reduced pressure to give 4-((1-(4-(2-(2-aminopyridin-3-yl)-5-(4-cyanophenyl)-3H-imidazo[4,5-b]pyridin-3-yl)benzyl)piperidin-4-yl)amino)pyrimidine-2-carbonitrile (Example 320, 42.6 mg, yield: 30%) as a yellow solid. MS: m/z=604.2 [M+H]⁺. ¹H NMR (400 MHz, Methanol-d₄) δ 8.27-8.22 (m, 3H), 8.05 (d, J=8.4 Hz, 1H), 8.03-7.97 (m, 2H), 7.79 (d, J=8.8 Hz, 2H), 7.56 (d, J=8.4 Hz, 2H), 7.46 (d, J=8.4 Hz, 2H), 7.34 (dd, J=7.6, 1.6 Hz 1H), 6.65-6.57 (m, 1H), 6.48 (dd, J=7.6, 5.2 Hz, 1H), 4.02-3.92 (m, 1H), 3.68 (s, 2H), 3.03-2.95 (m, 2H), 2.33-2.25 (m, 2H), 2.03-2.00 (m, 1H), 1.69-1.54 (m, 3H).

### Example 321: 2-((1-(4-(2-(2-Aminopyridin-3-yl)-5-(4-cyanophenyl)-3H-imidazo[4,5-b]pyridin-3-yl)benzyl)piperidin-4-yl)amino)pyrimidine-4-carbonitrile

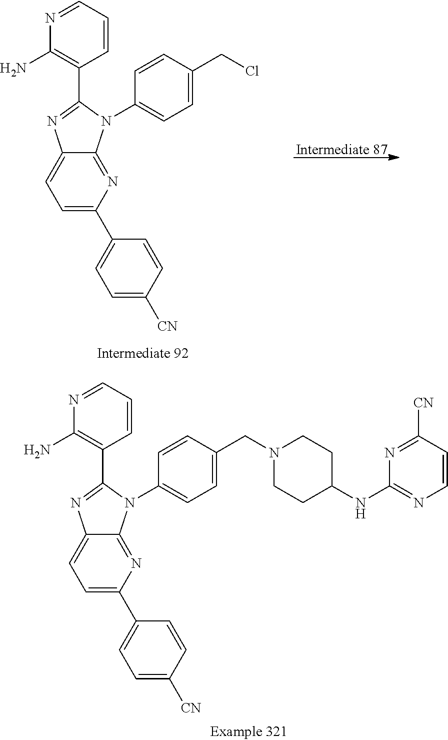 [SMILES: N#Cc1ccc(-c2ccc3nc(-c4cccnc4N)n(-c4ccc(CCl)cc4)c3n2)cc1 ; N#Cc1ccc(-c2ccc3nc(-c4cccnc4N)n(-c4ccc(CN5CCC(Nc6nccc(C#N)n6)CC5)cc4)c3n2)cc1]

To a solution of Intermediate 92 (100 mg, 229 μmol) and Intermediate 87 (46.5 mg, 229 μmol) in DMF (2 mL) were added K₂CO₃ (94.9 mg, 687 μmol) and NaI (6.86 mg, 45.8 μmol). The mixture was stirred at 50° C. for 0.5 hr. The reaction mixture was filtered. After purified by prep-HPLC (column: Phenomenex Cia 150×25 mm×10 μm; mobile phase: [water (NH₄HCO₃)-ACN]: B: 43%-73%, 10 min), 2-((1-(4-(2-(2-aminopyridin-3-yl)-5-(4-cyanophenyl)-3H-imidazo[4,5-b]pyridin-3-yl)benzyl)piperidin-4-yl)amino)pyrimidine-4-carbonitrile (Example 321, 35 mg, yield: 43%) was obtained as a yellow solid. MS: m/z=604.4 [M+H]⁺. ¹H NMR (400 MHz, Chloroform-d) δ 8.44 (br s, 1H), 8.19-8.11 (m, 3H), 8.09 (dd, J=4.8, 1.6 Hz, 1H), 7.84 (d, J=8.4 Hz, 1H), 7.72 (d, J=8.4 Hz, 2H), 7.53 (d, J=8.4 Hz, 2H), 7.40 (d, J=8.4 Hz, 2H), 7.11 (dd, J=7.6, 1.6 Hz, 1H), 6.83 (d, J=4.8 Hz, 1H), 6.65 (br s, 2H), 6.37 (dd, J=7.6, 4.8 Hz, 1H), 5.34 (d, J=8.0 Hz, 1H), 3.98-3.84 (m, 1H), 3.65 (s, 2H), 2.94-2.88 (m, 2H), 2.36-2.23 (m, 2H), 2.14-2.05 (m, 2H), 1.68-1.62 (m, 2H).

### Example 322: 2-(4-(4-(2-(2-Aminopyridin-3-yl)-5-(2-cyanophenyl)-3H-imidazo[4,5-b]pyridin-3-yl)benzyl)piperazin-1-yl)pyrimidine-4-carbonitrile

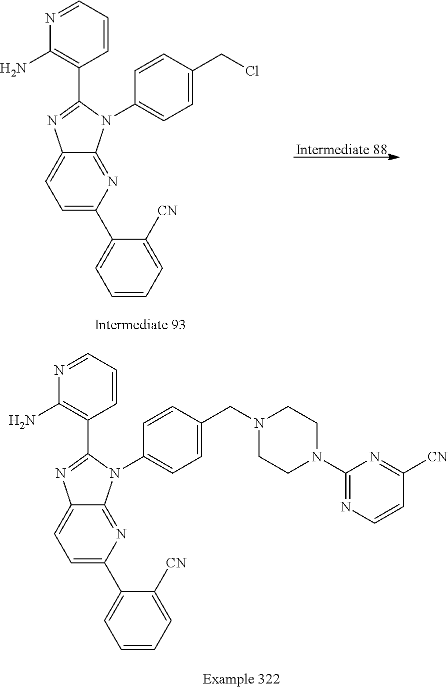 [SMILES: N#Cc1ccccc1-c1ccc2nc(-c3cccnc3N)n(-c3ccc(CCl)cc3)c2n1 ; N#Cc1ccnc(N2CCN(Cc3ccc(-n4c(-c5cccnc5N)nc5ccc(-c6ccccc6C#N)nc54)cc3)CC2)n1]

To a solution of Intermediate 93 (200 mg, 458 μmol) and Intermediate 86 (95.3 mg, 503 μmol) in DMF (3 mL) were added NaI (13.7 mg, 91.5 μmol), K₂CO₃ (189 mg, 1.37 mmol). The mixture was stirred at 80° C. for 12 hr. The reaction mixture was filtered and purified by prep-HPLC (column: Waters xbridge 150*25 mm*10 μm; mobile phase. [water (NH₄HCO₃)-ACN]; gradient: 50%-70% B over 14 min) to give 2-(4-(4-(2-(2-aminopyridin-3-yl)-5-(2-cyanophenyl)-3H-imidazo[4,5-b]pyridin-3-yl)benzyl)piperazin-1-yl)pyrimidine-4-carbonitrile (Example 322, 81 mg, yield: 30%) as a light-yellow solid. MS: m/z=590.2 [M+H]⁺. ¹H NMR (400 MHz, Dimethylsulfoxide-d₆) δ 8.62 (d, J=4.4 Hz, 1H), 8.38 (d, J=8.0 Hz, 1H), 8.02-8.00 (m, 1H), 7.63 (d, J=7.6 Hz, 1H), 7.87-7.80 (m, 3H), 7.61-7.58 (m, 1H), 7.51-7.44 (m, 4H), 7.19-7.10 (m, 2H), 6.99 (br s, 2H), 6.40 (dd, J=7.6, 4.8 Hz, 1H), 3.76-3.74 (m, 4H), 3.46 (s, 2H), 2.50-2.46 (m, 4H).

### Example 323: 4-((1-(4-(2-(2-Aminopyridin-3-yl)-5-(5-oxo-1,4-diazepan-1-yl)-3H-imidazo[4,5-b]pyridin-3-yl)benzyl)piperidin-4- yl)amino)pyrimidine-2-carbonitrile

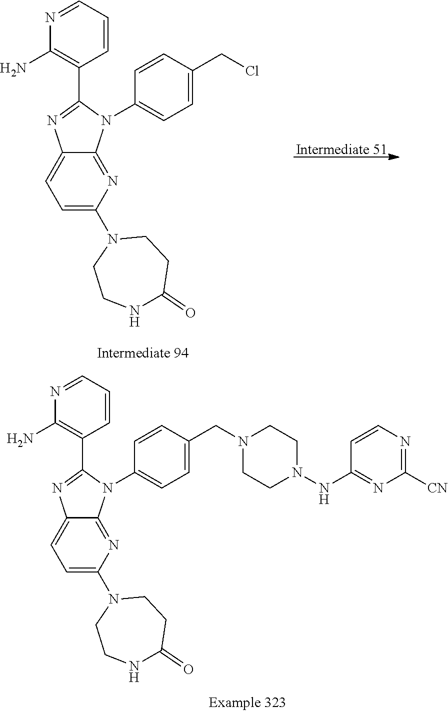 [SMILES: Nc1ncccc1-c1nc2ccc(N3CCNC(=O)CC3)nc2n1-c1ccc(CCl)cc1 ; N#Cc1nccc(NN2CCN(Cc3ccc(-n4c(-c5cccnc5N)nc5ccc(N6CCNC(=O)CC6)nc54)cc3)CC2)n1]

To a solution of Intermediate 94 (52.2 mg, 116 μmol) in DMF (3 mL) were added Intermediate 51 (26.0 mg, 128 μmol) and DIEA (60.2 mg, 81 ILL). The mixture was stirred at 80° C. for 12 hr and then concentrated. The residue was purified by prep-HPLC column: Boston Prime C18 150×30 mm×5 μm; mobile phase: [water (NH₃H₂O+NH₄HCO₃)-ACN]; gradient: B: 35%-65%, 7 min to give 4-((1-(4-(2-(2-aminopyridin-3-yl)-5-(5-oxo-1,4-diazepan-1-yl)-3H-imidazo[4,5-b]pyridin-3-yl)benzyl)piperidin-4-yl)amino)pyrimidine-2-carbonitrile (Example 323, 7.1 mg, yield: 9.9%) as a white solid. MS: m/z=615.5 [M+H]⁺. ¹H NMR (400 MHz, Dimethylsulfoxide-d₆) 8.92-7.81 (m, 4H), 7.59-7.55 (m, 1H), 7.51-7.22 (m, 4H), 7.14-6.94 (m, 3H), 6.87 (d, J=7.6 Hz, 1H), 6.67-6.66 (m, 1H), 6.32-6.31 (m, 1H), 3.92-3.62 (m, 5H), 3.56 (s, 2H), 3.21-3.09 (m, 2H), 2.81-2.78 (m, 2H), 2.38-2.49 (m, 2H), 2.13-2.11 (m, 2H), 1.92-1.81 (m, 2H), 1.56-1.41 (m, 2H).

### Example 324: 4-(4-(4-(2-(2-Aminopyridin-3-yl)-5-(2-chlorophenyl)-3H-imidazo[4,5-b]pyridin-3-yl)benzyl)piperazin-1-yl)pyrimidine-2-carbonitrile

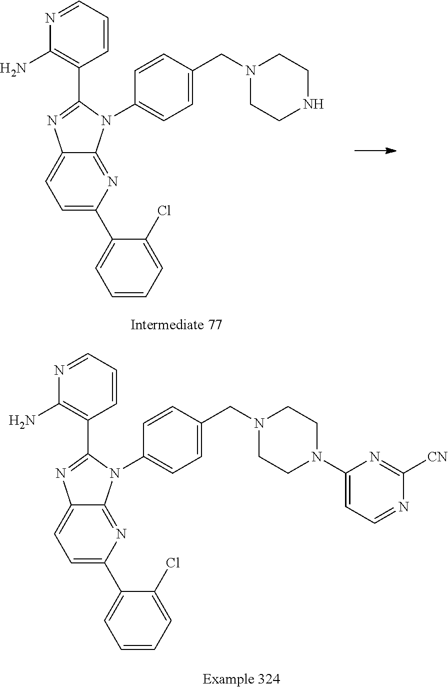 [SMILES: Nc1ncccc1-c1nc2ccc(-c3ccccc3Cl)nc2n1-c1ccc(CN2CCNCC2)cc1 ; N#Cc1nccc(N2CCN(Cc3ccc(-n4c(-c5cccnc5N)nc5ccc(-c6ccccc6Cl)nc54)cc3)CC2)n1]

A mixture of Intermediate 77 (28.1 mg, 202 μmol) and DIEA (52.1 mg, 403 μmol) in NMP (2 mL) were taken up into a microwave tube. The sealed tube was heated at 130° C. for Ihr under microwave. The reaction mixture was filtered and concentrated under reduced pressure. The residue was purified by prep-HPLC (column: Waters xbridge 150*25 mm 10 μm; mobile phase: [water (NH₄HCO₃)-ACN]; gradient: 49%-79% B over 10 min) to give 4-(4-(4-(2-(2-Aminopyridin-3-yl)-5-(2-chlorophenyl)-3H-imidazo[4,5-b]pyridin-3-yl)benzyl)piperazin-1-yl)pyrimidine-2-carbonitrile (Example 324, 34 mg, 28.2% yield) as a light-yellow solid. MS: m/z=599.1 [M+H]⁺. ¹H NMR (400 MHz, Dimethylsulfoxide-d₆) δ 8.30-8.25 (m, 2H), 8.00 (dd, J=4.8, 2.0 Hz, 1H), 7.63 (d, J=8.0 Hz, 1H), 7.58-7.53 (m, 2H), 7.47-7.42 (m, 6H), 7.15 (dd, J=7.6, 2.0 Hz, 1H), 7.09 (d, J=6.4 Hz, H), 6.96 (br s, 2H), 6.37 (dd, 1=7.6, 4.8 Hz, 1H), 3.72-3.58 (m, 6H), 2.49-2.46 (m, 4H).

### Example 325: 2-(4-(4-(2-(2-Aminopyridin-3-yl)-5-(2-chlorophenyl)-3H-imidazo[4,5-b]pyridin-3-yl)benzyl)piperazin-1-yl)pyrimidine-4-carbonitrile

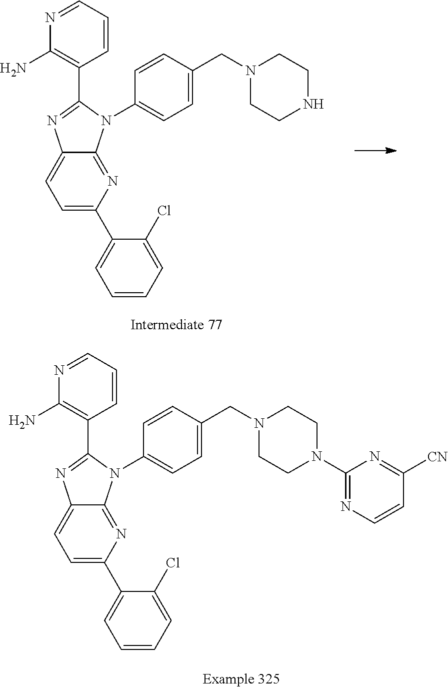 [SMILES: Nc1ncccc1-c1nc2ccc(-c3ccccc3Cl)nc2n1-c1ccc(CN2CCNCC2)cc1 ; N#Cc1ccnc(N2CCN(Cc3ccc(-n4c(-c5cccnc5N)nc5ccc(-c6ccccc6Cl)nc54)cc3)CC2)n1]

A mixture of Intermediate 77 (100 mg, 202 μmol) and 2-chloropyrimidine-4-carbonitrile (28.1 mg, 202 μmol) and DIEA (78.2 mg, 605 μmol) in NMP (2 mL) was taken up into a microwave tube. The sealed tube was heated at 130° C. for 1 hr under microwave. The reaction mixture was filtered and purified by prep-HPLC (column: Phenomenex C₁₈ 150×25 mm×10 μm; mobile phase: [water (NH₄HCO₃)-ACN]; B %: 48%-78%, over 14 min) to give 2-(4-(4-(2-(2-aminopyridin-3-yl)-5-(2-chlorophenyl)-3H-imidazo[4,5-b]pyridin-3-yl)benzyl)piperazin-1-yl)pyrimidine-4-carbonitrile (Example 325, 35.8 mg, yield: 29%) as a yellow solid. MS: m/z=599.3 [M+H]⁺. ¹H NMR (400 MHz, Dimethylsulfoxide-d₆) δ 8.63 (d, J=4.8 Hz, 1H), 8.29 (d, J=8.4 Hz, 1H), 8.03-7.99 (m, 1H), 7.63 (d, J=8.4 Hz, 1H), 7.59-7.53 (m, 2H), 7.48-7.42 (m, 6H), 7.18-7.13 (m, 2H), 6.97 (br s, 2H), 6.38 (dd, J=8.0, 4.8 Hz, 1H), 3.83-3.73 (m, 4H), 3.59 (s, 2H), 2.49-2.44 (m, 4H).

### Example 326: 6-(4-(4-(2-(2-Aminopyridin-3-yl)-5-(2-chlorophenyl)-3H-imidazo[4,5-b]pyridin-3-yl)benzyl)piperazin-1-yl)pyrimidine-4-carbonitrile

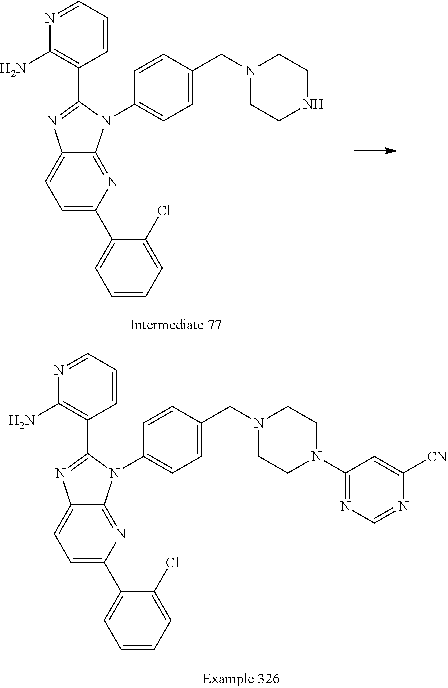 [SMILES: Nc1ncccc1-c1nc2ccc(-c3ccccc3Cl)nc2n1-c1ccc(CN2CCNCC2)cc1 ; N#Cc1cc(N2CCN(Cc3ccc(-n4c(-c5cccnc5N)nc5ccc(-c6ccccc6Cl)nc54)cc3)CC2)ncn1]

A mixture of Intermediate 77 (100 mg, 302 μmol), 6-chloropyrimidine-4-carbonitrile (28.1 mg, 202 μmol) and DIEA (78.2 mg, 605 μmol) in NMP (2 mL) was taken up into a microwave tube. The sealed tube was heated at 130° C. for 1 hr under microwave. The reaction mixture was filtered to give a filter liquid. After purified by prep-HPLC (column: Phenomenex C₁a 150×25 mm×10 μm; mobile phase: [water (NH₄HCO₃)-ACN]; B %: 40%-70%, over 14 min), 6-(4-(4-(2-(2-aminopyridin-3-yl)-5-(2-chlorophenyl)-3H-imidazo[4,5-b]pyridin-3-yl)benzyl)piperazin-1-yl)pyrimidine-4-carbonitrile (Example 326, 26.4 mg, yield: 21%) was obtained as a yellow lyophilized powder. MS: m/z=599.4 [M+H]⁺. ¹H NMR (400 MHz, Dimethylsulfoxide-d₆) δ 8.55 (s, 1H), 8.29 (d, J=8.4 Hz, 1H), 8.00 (dd, J=4.8, 1.6 Hz, 1H), 7.63 (d, J=8.4 Hz, 1H), 7.59-7.53 (m, 3H), 7.48-7.41 (m, 6H), 7.16 (dd, J=7.6, 1.6 Hz, 1H), 6.97 (br s, 2H), 6.36 (dd, J=7.6, 4.8 Hz, JH), 3.85-3.62 (m, 4H), 3.59 (s, 2H), 2.48-2.44 (m, 4H).

### Example 327: 5-(4-(4-(2-(2-Aminopyridin-3-yl)-5-phenyl-3H-imidazo[4,5-b]pyridin-3-yl)benzyl)piperazine-1-carbonyl)thiazole-2-carbonitrile

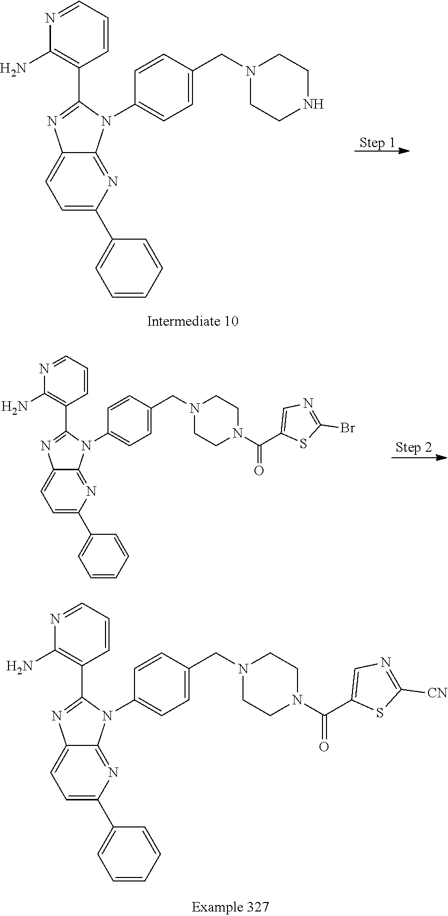

### Step 1: (4-(4-(2-(2-Aminopyridin-3-yl)-5-phenyl-3H-imidazo[4,5-b]pyridin-3-yl)benzyl)piperazin-1-yl)(2-bromothiazol-5-yl)methanone

To a solution of Intermediate 10 (100 mg, 217 μmol) and 2-bromothiazole-5-carboxylic acid (49.6 mg, 238 μmol) in CH₂Cl₂ (3 mL) was added HATU (98.9 mg, 260 μmol), followed by addition of DIEA (84.0 mg, 650 μmol). The resulting mixture was stirred at 25° C. for 2 hr. Then the mixture was added to water (3 mL) and extracted with EtOAc (5 mL×3). The combined organic layers were washed with brine (5 mL), dried over anhydrous Na₂SO₄, filtered and concentrated. The crude was purified by silica gel flash chromatography (Eluent of 0-8% MeOH in CH₂Cl₂) to give (4-(4-(2-(2-Aminopyridin-3-yl)-5-phenyl-3H-imidazo[4,5-b]pyridin-3-yl)benzyl)piperazin-1-yl)(2-bromothiazol-5-yl)methanone (70 mg, yield: 50%) as a yellow solid. MS: m/z=651.4, 652.3 [M+H]⁺. ¹H NMR (400 MHz, Chloroform-d) δ 8.17 (d, J=8.0 Hz, 1H), 8.02-8.00 (m, 3H), 7.84 (d, J=8.0 Hz, 1H), 7.71 (s, 1H), 7.52-7.49 (m, 2H) 7.46-7.39 (m, 6H), 6.42 (dd, J=8.0, 5.6 Hz, 1H), 3.82-3.76 (m, 4H), 3.69 (s, 2H), 2.60-2.54 (m, 4H).

### Step 2: 5-(4-(4-(2-(2-Aminopyridin-3-yl)-5-phenyl-3H-imidazo[4,5-b]pyridin-3-yl)benzyl)piperazine-1-carbonyl)thiazole-2-carbonitrile

To a solution of (4-(4-(2-(2-aminopyridin-3-yl)-5-phenyl-3H-imidazo[4,5-b]pyridin-3-yl)benzyl)piperazin-1-yl)(2-bromothiazol-5-yl)methanone (100 mg, 153 μmol) in pyridine (4 mL) was added CuCN (29 mg, 322 μmol) at 25° C. The mixture was stirred at 145° C. for 6 hr. Then the mixture was filtered and concentrated under reduced pressure. The residue was purified by prep-HPLC (column: Waters xbridge 150*25 mm 10 μm; mobile phase: [water (NH₄HCO₃)-ACN]; gradient: 40%-70% B over 10 min) to give 5-(4-(4-(2-(2-aminopyridin-3-yl)-5-phenyl-3H-imidazo[4,5-b]pyridin-3-yl)benzyl)piperazine-1-carbonyl)thiazole-2-carbonitrile. (Example 327, 8 mg, 8.7% yield) as a light-yellow solid. MS: m/z=598.3 [M+H]r. ¹H NMR (400 MHz, Acetonitrile-d₃) δ 8.23-8.18 (m, 2H), 8.06-8.04 (m, 3H), 7.92 (d, J=8.0 Hz, 1H), 7.57-7.43 (m, 7H), 7.17 (dd, J=7.6, 1.6 Hz, 1H), 6.60 (b rs, 2H), 6.40 (dd, J=7.6, 4.8 Hz, 1H), 3.75-3.67 (m, 6H), 2.59-2.52 (m, 4H).

### Example 328: 4-(4-(4-(2-(2-Aminopyridin-3-yl)-5-phenyl-3H-imidazo[4,5-b]pyridin-3-yl)benzyl)piperazin-1-yl)-1,3,5-triazine-2-carbonitrile

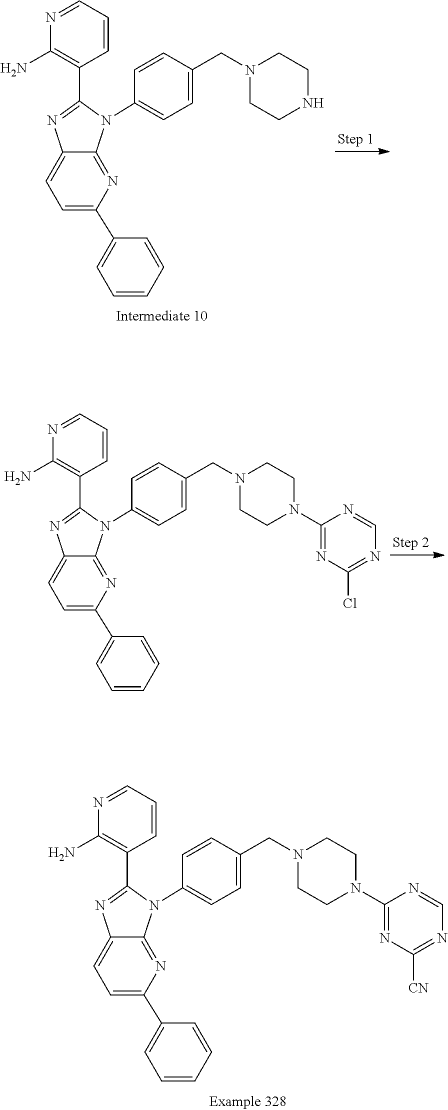 [SMILES: Nc1ncccc1-c1nc2ccc(-c3ccccc3)nc2n1-c1ccc(CN2CCNCC2)cc1 ; Nc1ncccc1-c1nc2ccc(-c3ccccc3)nc2n1-c1ccc(CN2CCN(c3ncnc(Cl)n3)CC2)cc1 ; N#Cc1ncnc(N2CCN(Cc3ccc(-n4c(-c5cccnc5N)nc5ccc(-c6ccccc6)nc54)cc3)CC2)n1]

### Step 1: 3-(3-(4-((4-(4-Chloro-1,3,5-triazin-2-yl)piperazin-1-yl)methyl)phenyl)-5-phenyl-3H-imidazo[4,5-b]pyridin-2-yl)pyridin-2-amine

To a solution of Intermediate 10 (200 mg, 433 μmol) in CH₂Cl₂ (5 mL) was added 2,4-dichloro-1,3,5-triazine (65.0 mg, 433 μmol), followed by addition of DIEA (168 mg, 1.30 mmol) at 0° C. The reaction mixture was stirred at 0° C. for 1 hr and concentrated under reduced pressure. The residue was purified by prep-TLC (CH₂Cl₂:MeOH=10:1) to give 3-(3-(4-((4-(4-chloro-1,3,5-triazin-2-yl)piperazin-1-yl)methyl)phenyl)-5-phenyl-3H-imidazo[4,5-b]pyridin-2-yl)pyridin-2-amine (30 mg, yield: 12%) as a white solid. MS: m/z=575.2 [M+H]⁺. ¹H NMR (400 MHz, Chloroform-d) 8.37 (s, 1H), 8.19 (d, J=8.0 Hz, 1H), 8.01 (d, J=8.0 Hz, 2H), 7.93 (d, J=4.0 Hz, 1H), 7.85 (d, J=8.4 Hz, 1H), 7.62-7.58 (m, 2H), 7.54-7.49 (m, 1H), 7.46-7.44 (m, 4H), 7.42-7.40 (m, 1H), 6.47-6.42 (m, 1H), 4.02-3.95 (m, 4H), 3.73 (s, 2H), 2.70-2.61 (m, 4H).

### Step 2: 4-(4-(4-(2-(2-Aminopyridin-3-yl)-5-phenyl-3H-imidazo[4,5-b]pyridin-3-yl)benzyl)piperazin-1-yl)-1,3,5-triazine-2-carbonitrile

To a mixture of 3-(3-(4-((4-(4-chloro-1,3,5-triazin-2-yl)piperazin-1-yl)methyl)phenyl)-5-phenyl-3H-imidazo[4,5-b]pyridin-2-yl)pyridin-2-amine (30 mg, 52.2 μmol) in DMSO (1 mL) were added KCN (10 mg, 154 μmol) and DABCO (585 μg, 5.22 μmol). The mixture was stirred at 60° C. for 1 hr. H₂O (20 ml) was added to the mixture and extracted with EtOAc (20 mL×3), washed with brine (20 mL), dried over Na₂SO₄, filtered, and concentrated and purified by prep-HPLC (column: Phenomenex C18 80×40 mm×3 μm; mobile phase: [water (NH₄HCO₃)-ACN]; B %: 54%-84%, 8 min) to give 4-(4-(4-(2-(2-aminopyridin-3-yl)-5-phenyl-3H-imidazo[4,5-b]pyridin-3-yl)benzyl)piperazin-1-yl)-1,3,5-triazine-2-carbonitrile (Example 328, 1.1 mg, yield: 5.5%) as a white solid. MS: m/z=566.3 [M+H]⁺. ¹H NMR (400 MHz, Chloroform-d) 8.56 (s, 1H), 8.15 (d, J=8.0 Hz, 1H), 8.05-8.01 (m, 3H), 7.82 (d, J=8.0 Hz, 1H), 7.53-7.51 (m, 2H), 7.45-7.43 (m, 4H), 7.41-7.39 (m, 1H), 7.13 (d, J=4.0 Hz, 1H), 6.91 (br s, 2H), 6.40-6.36 (m, 1H), 3.97-3.92 (m, 4H), 3.67 (s, 2H), 2.61-2.57 (m, 4H).

### Example 329: 4-(4-(4-(2-(2-Aminopyridin-3-yl)-5-(cyclohex-1-en-1-yl)-3H-imidazo[4,5-b]pyridin-3-yl)benzyl)piperazin-1-yl)pyrimidine-2-carbonitrile

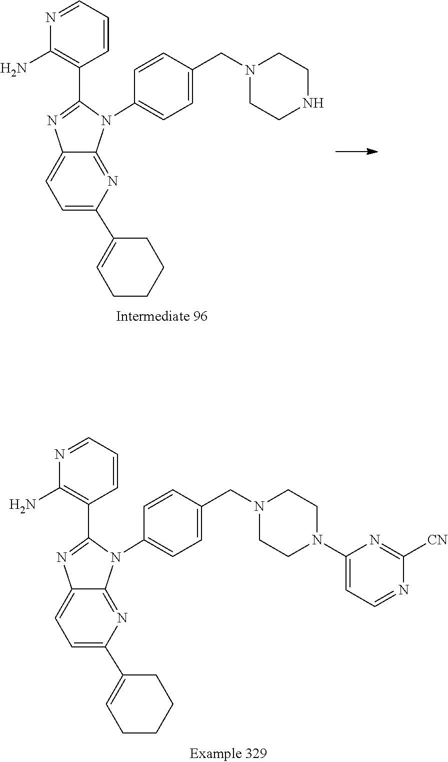 [SMILES: Nc1ncccc1-c1nc2ccc(C3=CCCCC3)nc2n1-c1ccc(CN2CCNCC2)cc1 ; N#Cc1nccc(N2CCN(Cc3ccc(-n4c(-c5cccnc5N)nc5ccc(C6=CCCCC6)nc54)cc3)CC2)n1]

To a solution of Intermediate 96 (100 mg, HCl salt, 199 μmol) and 4-chloropyrimidine-2-carbonitrile (27.8 mg, 199 μmol) in NMP (4 mL) was added DIEA (129 mg, 173 μL). The mixture was stirred at 160° C. for Ihr under microwave. The mixture was quenched with water (10 mL) at 0° C. and extracted with EtOAc (20 mL×3). The combined organic phases were washed with brine (30 mL), dried over anhydrous Na₂SO₄, filtered and concentrated. The residue was purified by prep-HPLC (column: Phenomenex C18 75×30 mm×3 μm; mobile phase: [water (NH₃H₂O+NH₄HCO₃)-ACN]; gradient: 60%-90% B over 8 min) to give 4-(4-(4-(2-(2-aminopyridin-3-yl)-5-(cyclohex-1-en-1-yl)-3H-imidazo[4,5-h]pyridin-3-yl)benzyl)piperazin-1-yl)pyrimidine-2-carbonitrile (Example 329, 20.8 mg, yield: 21%) as a yellow solid. MS: m/z=569.2 [M+H]⁺. ¹H NMR (400 MHz, Dimethylsulfoxide-d₆) δ 8.27 (d, J=6.4 Hz, 1H), 8.11 (d, J=8.0 Hz, 1H), 8.02-7.94 (m, 1H), 7.53 (d, J=8.4 Hz, 1H), 7.48 (d, J=8.0 Hz, 2H), 7.38 (d, J=8.4 Hz, 2H), 7.15-7.07 (m, 2H), 7.03 (br s, 2H), 6.68-6.60 (m, 1H), 6.34 (dd, J=7.6, 4.8 Hz, 1H), 3.77-3.57 (m, 6H), 3.31 (s, 2H), 2.45-2.39 (m, 4H), 2.20-2.18 (m, 2H), 1.72-1.56 (m, 4H).

### Example 330: 4-(4-(4-(2-(2-Aminopyridin-3-yl)-5-(3,6-dihydro-2H-pyran-4-yl)-3H-imidazo[4,5-b]pyridin-3-yl)benzyl)piperazin-1-yl)pyrimidine-2-carbonitrile

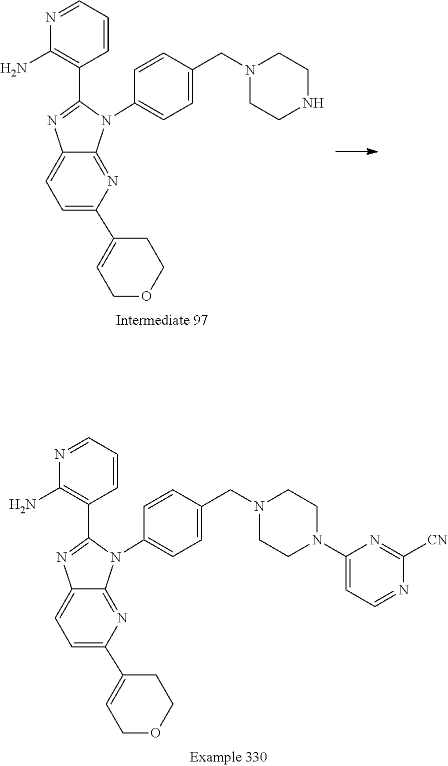 [SMILES: Nc1ncccc1-c1nc2ccc(C3=CCOCC3)nc2n1-c1ccc(CN2CCNCC2)cc1 ; N#Cc1nccc(N2CCN(Cc3ccc(-n4c(-c5cccnc5N)nc5ccc(C6=CCOCC6)nc54)cc3)CC2)n1]

To a solution of Intermediate 97 (100 mg, HCl salt, 198 μmol) and 4-chloropyrimidine-2-carbonitrile (27.7 mg, 198 μmol) in NMP (4 mL) was added DIEA (128 mg, 992 μmol, 173 L). The mixture was stirred at 160° C. for 1 hr under microwave. The mixture was quenched with water (10 mL) at 0° C. and then phase was extracted with EtOAc (20 mL×3). The combined organic layers were washed with brine (30 mL), dried over anhydrous Na₂SO₄, filtered and concentrated. The residue was purified by prep-HPLC (column: Boston Prime C18 150×30 mm×5 μm; mobile phase: [water (NH₃H₂O+NH₄HCO₃)-ACN]; gradient: 53%-83% B over 7 min) to give 4-(4-(4-(2-(2-aminopyridin-3-yl)-5-(3,6-dihydro-2H-pyran-4-yl)-3H-imidazo[4,5-b]pyridin-3-yl)benzyl)piperazin-1-yl)pyrimidine-2-carbonitrile. (Example 330, 18.9 mg, yield: 19%) as a yellow solid. MS: m/z=571.1 [M+H]⁺. ¹H NMR (400 MHz, Dimethylsulfoxide-d₆) δ 8.27 (d, J=6.0 Hz, 1H), 8.16 (d, J=8.4 Hz, 1H), 7.98 (dd, J=4.8, 1.6 Hz, 1H), 7.59 (d, J=8.0 Hz, 1H), 7.48 (d, J=8.4 Hz, 2H), 7.39 (d, J=8.4 Hz, 2H), 7.14-7.08 (m, 2H), 7.02 (br s, 2H), 6.69-6.68 (m, 1H), 6.35 (dd, J=7.6, 4.8 Hz, 1H), 4.28-4.22 (m, 2H), 3.80 (t, J=5.6 Hz, 2H), 3.75-3.58 (m, 6H), 3.32-3.31 (m, 6H).

### Example 331: 4-(4-(4-(2-(2-Aminopyridin-3-yl)-5-(3-cyanophenyl)-3H-imidazo[4,5-b]pyridin-3-yl)benzyl)piperazin-1-yl)pyrimidine-2-carbonitrile

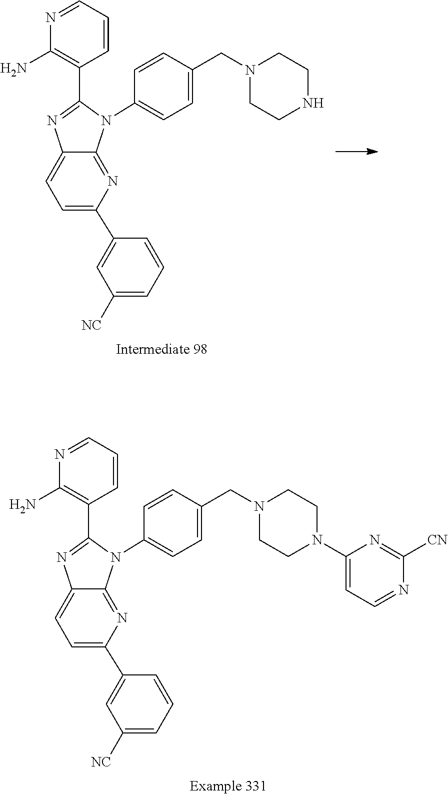 [SMILES: N#Cc1cccc(-c2ccc3nc(-c4cccnc4N)n(-c4ccc(CN5CCNCC5)cc4)c3n2)c1 ; N#Cc1cccc(-c2ccc3nc(-c4cccnc4N)n(-c4ccc(CN5CCN(c6ccnc(C#N)n6)CC5)cc4)c3n2)c1]

A mixture of Intermediate 98 (100 mg, 206 μmol), 4-chloropyrimidine-2-carbonitrile (29 mg, 210 μmol) and DIEA (136 mg, 1.05 mmol) in NMP (3 mL) was taken up into a microwave tube. The sealed tube was heated at 130° C. for 1 hr under microwave. The reaction mixture was quenched with H₂O (5 mL) at 0° C. and extracted with CH₂Cl₂ (15 mL×2). The combined organic layers were washed with brine (15 mL), dried over Na₂SO₄, filtered, and concentrated under reduced pressure. After purified by prep-HPLC (column: Waters xbridge 150*25 mm 10 μm; mobile phase: [water (NH₄HCO₃)-ACN]; gradient: 45%-75% B over 10 min), 4-(4-(4-(2-(2-aminopyridin-3-yl)-5-(3-cyanophenyl)-3H-imidazo[4,5-b]pyridin-3-yl)benzyl)piperazin-1-yl)pyrimidine-2-carbonitrile (Example 331, 37.6 mg, yield: 29%) was obtained as a brown solid. MS: m/z=590.4 [M+H]⁺. ¹H NMR (400 MHz, Dimethylsulfoxide-d₆) δ 8.48-8.43 (m, 1H), 8.38 (d, J=8.4 Hz, 1H), 8.33 (d, J=8.4 Hz, 1H), 8.27 (d, J=6.4 Hz, 1H), 8.12 (d, J=8.4 Hz, 1H), 8.01 (dd, J=4.8, 2.0 Hz, 1H), 7.87 (d, J=7.6 Hz, 1H), 7.69 (dd, J=7.6, 7.6 Hz, 1H), 7.54-7.47 (m, 4H), 7.16 (dd, J=7.6, 1.6 Hz, 1H), 7.11 (d, J=6.8 Hz, 1H), 7.05 (br s, 2H), 6.37 (dd, J=7.6, 4.8 Hz, 1H), 3.70-3.69 (m, 4H), 3.65 (s, 2H), 2.49-2.47 (m, 4H).

### Example 332: 4-(4-(4-(2-(2-Aminopyridin-3-yl)-5-(6-oxo-1,6-dihydropyridin-3-yl)-3H-imidazo[4,5-b]pyridin-3-yl)benzyl)piperazin-1-yl)pyrimidine-2-carbonitrile

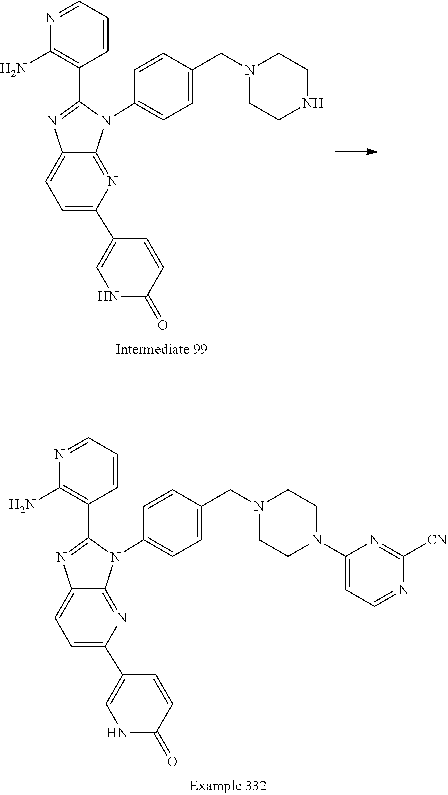 [SMILES: Nc1ncccc1-c1nc2ccc(-c3ccc(=O)[nH]c3)nc2n1-c1ccc(CN2CCNCC2)cc1 ; N#Cc1nccc(N2CCN(Cc3ccc(-n4c(-c5cccnc5N)nc5ccc(-c6ccc(=O)[nH]c6)nc54)cc3)CC2)n1]

A mixture of Intermediate 99 (120 mg, 251 μmol), 4-chloropyrimidine-2-carbonitrile (35 mg, 206 μmol) and DIEA (162 mg, 1.25 mmol) in NMP (3 mL) was taken up into a microwave tube. The sealed tube was heated at 130° C. for 1 hr under microwave. The reaction mixture was quenched with H₂O (5 mL) at 0° C. and extracted with CH₂Cl₂ (15 mL×3). The combined organic layers were washed with brine (30 mL), dried over Na₂SO₄, filtered and concentrated under reduced pressure. After purified by prep-HPLC (column: Waters xbridge 150*25 mm 10 μm; mobile phase: [water (NH₄HCO₃)-ACN]; gradient: 20%-50% B over 10 min)), 4-(4-(4-(2-(2-aminopyridin-3-yl)-5-(6-oxo-1,6-dihydropyridin-3-yl)-3H-imidazo[4,5-b]pyridin-3-yl)benzyl)piperazin-1-yl)pyrimidine-2-carbonitrile (Example 332, 16 mg, yield: 10%) was obtained as a yellow solid. MS: m/z=582.2 [M+H]⁺. ¹H NMR (400 MHz, Dimethylsulfoxide-d₆) δ 12.01-11.61 (m, 1H), 8.27 (d, J=6.0 Hz, 1H), 8.20 (d, J=8.0 Hz, 1H), 8.13 (dd, J=9.6, 2.8 Hz, 1H), 8.08-8.03 (m, 1H), 7.98 (dd, J=4.4, 1.2 Hz, 1H), 7.84 (d, J=8.4 Hz, 1H), 7.53-7.41 (m, 4H), 7.15-7.09 (m, 2H), 7.04 (br s, 2H), 6.43 (d, J=9.6 Hz, 1H), 6.35 (dd, J=8.0, 5.2 Hz, 1H), 3.78-3.66 (m, 4H), 3.64 (s, 2H), 2.52-2.51 (m, 4H).

### Example 333: 2-(4-(4-(2-(2-Aminopyridin-3-yl)-5-(6-oxo-1,6-dihydropyridin-2-yl)-3H-imidazo[4,5-b]pyridin-3-yl)benzyl)piperazin-1-yl)pyrimidine-4-carbonitrile

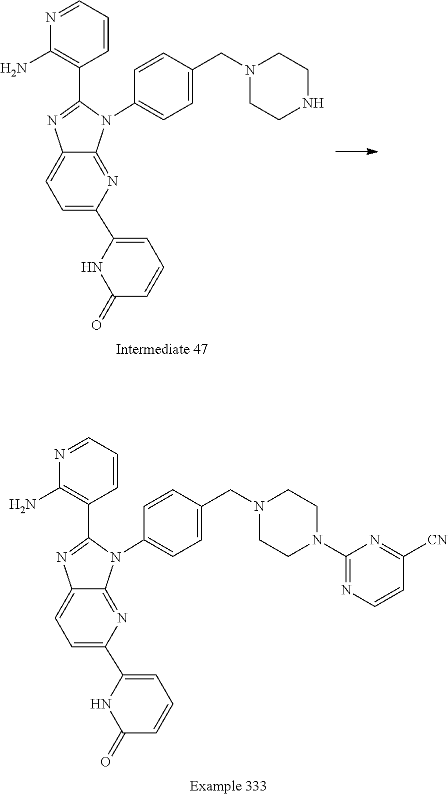 [SMILES: Nc1ncccc1-c1nc2ccc(-c3cccc(=O)[nH]3)nc2n1-c1ccc(CN2CCNCC2)cc1 ; N#Cc1ccnc(N2CCN(Cc3ccc(-n4c(-c5cccnc5N)nc5ccc(-c6cccc(=O)[nH]6)nc54)cc3)CC2)n1]

A mixture of Intermediate 47 (149 mg, 312 μmol), 2-chloropyrimidine-4-carbonitrile (43.6 mg, 312 μmol) and DIEA (101 mg, 780 μmol) in NMP (2.5 mL) was taken up into a microwave tube. The sealed tube was heated at 130° C. for 1 hr under microwave. The reaction was filtered and concentrated under reduced pressure. The crude was purified by prep-HPLC (column: Waters xbridge 150*25 mm 10 μm; mobile phase: [water (NH₄HCO₃)-ACN]; gradient: 29%-59% B over 10 min) to give 2-(4-(4-(2-(2-aminopyridin-3-yl)-5-(6-oxo-1,6-dihydropyridin-2-yl)-3H-imidazo[4,5-b]pyridin-3-yl)benzyl)piperazin-1-yl)pyrimidine-4-carbonitrile (Example 333, 15.8 mg, yield: 8.7%) as a yellow solid. MS: m/z=582.2 [M+H]⁺. ¹H NMR (400 MHz, Dimethylsulfoxide-d₆) δ 10.81 (br s, 1H), 8.65 (d, J=4.8 Hz, 1H), 8.33 (d, J=8.4 Hz, 1H), 8.18-8.16 (m, 1H), 8.01 (dd, J=4.4, 1.2 Hz, 1H), 7.64-7.46 (m, 6H), 7.18-7.14 (m, 2H), 7.03 (br s, 2H), 6.50-6.43 (m, 1H), 6.38 (dd, J=7.6, 4.8 Hz, 1H), 3.82-3.76 (m, 4H), 3.65 (s, 2H), 2.65-2.55 (m, 4H).

### Example 334: 6-(4-(4-(2-(2-Aminopyridin-3-yl)-5-(6-oxo-1,6-dihydropyridin-2-yl)-3H-imidazo[4,5-b]pyridin-3-yl)benzyl)piperazin-1-yl)pyrimidine-4-carbonitrile

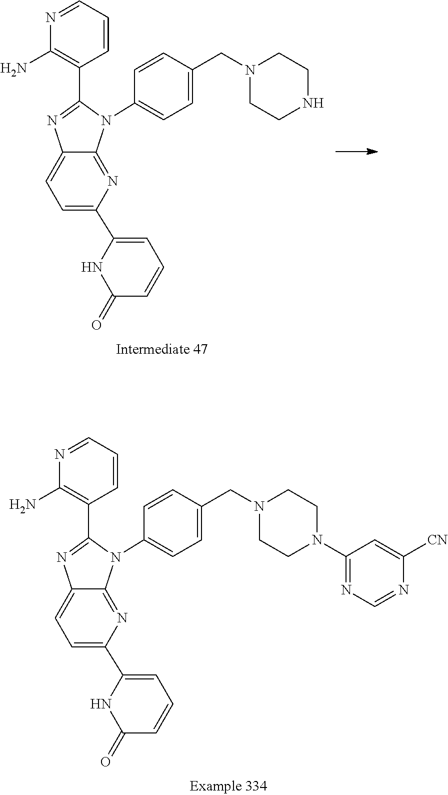 [SMILES: Nc1ncccc1-c1nc2ccc(-c3cccc(=O)[nH]3)nc2n1-c1ccc(CN2CCNCC2)cc1 ; N#Cc1cc(N2CCN(Cc3ccc(-n4c(-c5cccnc5N)nc5ccc(-c6cccc(=O)[nH]6)nc54)cc3)CC2)ncn1]

A mixture of Intermediate 47 (100 mg, 209 μmol), 6-Chloropyrimidine-4-carbonitrile (29.2 mg, 209 μmol), and DLEA (67.5 mg, 522 μmol) in NMP (2 mL) was taken up into a microwave tube. The sealed tube was heated at 130° C. for 1 hr under microwave. The mixture was filtered and concentrated under reduced pressure. The crude was purified by prep-HPLC (column: Waters xbridge 150*25 mm 10 μm; mobile phase: [water (NH₄HCO₃)-ACN]; gradient: 22%-52% B over 10 min) to give 6-(4-(4-(2-(2-aminopyridin-3-yl)-5-(6-oxo-1,6-dihydropyridin-2-yl)-3H-imidazo[4,5-b]pyridin-3-yl)benzyl)piperazin-1-yl)pyrimidine-4-carbonitrile (Example 334, 50 mg, yield: 41%) as a light-yellow solid. MS: m/z=582.3 [M+H]⁺. ¹H NMR (400 MHz, Dimethylsulfoxide-d₆) δ 10.80 (br s, 1H), 8.56 (s, 1H), 8.33 (d, J=8.0 Hz, 1H), 8.20-8.14 (m, 1H), 8.00 (dd, J=4.8, 2.0 Hz, 1H), 7.64-7.46 (m, 7H), 7.15 (dd, J=7.6, 2.0 Hz, 1H), 7.03 (br s, 2H), 6.47 (d, J=8.4 Hz, 1H), 6.37 (dd, J=7.6, 4.8 Hz, 1H), 3.82-3.76 (m, 4H), 3.65 (s, 2H), 2.53-2.52 (m, 4H).

### Example 335: 4-((1-(4-(2-(2-Aminopyridin-3-yl)-5-(2-cyanophenyl)-3H-imidazo[4,5-b]pyridin-3-yl)benzyl)piperidin-4-yl)amino)pyrimidine-2-carbonitrile

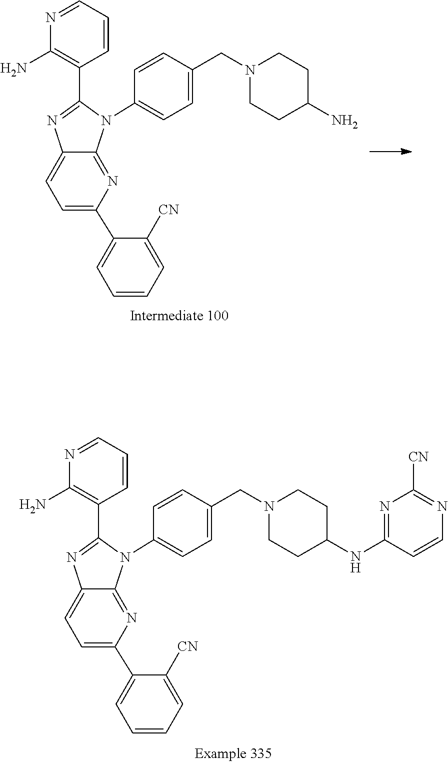 [SMILES: N#Cc1ccccc1-c1ccc2nc(-c3cccnc3N)n(-c3ccc(CN4CCC(N)CC4)cc3)c2n1 ; N#Cc1nccc(NC2CCN(Cc3ccc(-n4c(-c5cccnc5N)nc5ccc(-c6ccccc6C#N)nc54)cc3)CC2)n1]

To a solution of Intermediate 100 (80 mg, 160 μmol) in DMF (2 mL) were added 4-chloropyrimidine-2-carbonitrile (27 mg, 192 μmol), NaI (2.4 mg, 16 plmol) and K₂CO₃ (66 mg, 479 μmol). The mixture was stirred at 80° C. for 1 hr. The reaction mixture was poured into H₂O (15 mL) and extracted with EtOAc (45 mL×2). The combined organic layers were dried over anhydrous Na₂SO₄, filtered and concentrated under reduced pressure. The residue was purified by silica gel flash chromatography (Eluent of 0˜6% MeOH in CH₂Cl₂) to give 4-((1-(4-(2-(2-aminopyridin-3-yl)-5-(2-cyanophenyl)-3H-imidazo[4,5-b]pyridin-3-yl)benzyl)piperidin-4-yl)amino)pyrimidine-2-carbonitrile (Example 335, 24 mg, yield: 25%) as a yellow solid. MS: m/z=604.3 [M+H]f ¹H NMR (400 MHz, Methanol-d₄) δ 8.28 (d, J=8.4 Hz, 1H), 8.08-7.95 (m, 2H), 7.91-7.78 (m, 3H), 7.75-7.71 (m, 1H), 7.57-7.50 (m, 5H), 7.39-7.29 (m, 1H), 6.66-6.54 (m, 1H), 6.53-6.42 (m, 1H), 4.07-3.89 (m, 1H), 3.73 (s, 2H), 3.07-2.95 (m, 2H), 2.43-2.29 (m, 2H), 2.02-1.94 (m, 2H), 1.67-1.56 (m, 2H).

### Example 336: N-(1-(4-(2-(2-aminopyridin-3-yl)-5-phenyl-3H-imidazo[4,5-b]pyridin-3-yl)benzyl)piperidin-4-yl)-2-cyano-N-methylpyrimidine-4-carboxamide

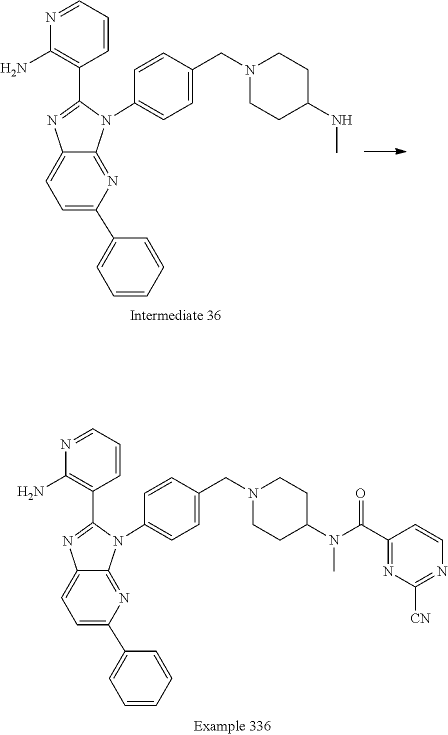 [SMILES: CNC1CCN(Cc2ccc(-n3c(-c4cccnc4N)nc4ccc(-c5ccccc5)nc43)cc2)CC1 ; CN(C(=O)c1ccnc(C#N)n1)C1CCN(Cc2ccc(-n3c(-c4cccnc4N)nc4ccc(-c5ccccc5)nc43)cc2)CC1]

To solution of Intermediate 36 (50 mg, 102 μmol), 2-cyanopyrimidine-4-carboxylic acid (19.8 mg, 133 μmol), EDCI (29.4 mg, 153 μmol) and HOBt (20.7 mg, 153 μmol) in CH₂Cl₂ (2 mL) was added DIEA (52.8 mg, 408 μmol). The mixture was stirred at 25° C. for 16 hr. The reaction mixture was diluted with H₂O (10 mL) and extracted with CH₂Cl₂ (20 mL×2). The combined organic layers were washed with brine (10 mL), dried over Na₂SO₄, filtered and concentrated under reduced pressure. The crude was purified byprep-TLC (CH₂Cl₂:MeOH=20:1) to give N-(1-(4-(2-(2-aminopyridin-3-yl)-5-phenyl-3H-imidazo[4,5-b]pyridin-3-yl)benzyl)piperidin-4-yl)-2-cyano-N-methylpyrimidine-4-carboxamide (Example 336, 24.3 mg, yield: 38%) as a yellow solid. MS: m/z=621.3 [M+H]⁺. ¹H NMR (400 MHz, Dimethylsulfoxide-d₆) δ 9.15 (dd, J=5.2, 3.2 Hz, 1H), 8.30-8.24 (m, 1H), 8.05-7.96 (m, 5H), 7.51-7.40 (m, 7H), 7.17-7.10 (m, 1H), 7.03 (d, J=4.8 Hz, 2H), 6.42-6.31 (m, 1H), 3.61-3.51 (m, 2H), 2.95-2.77 (m, 5H), 2.19-2.11 (m, 1H), 1.91-1.84 (m, 2H), 1.70-1.63 (m, 2H), 1.36-1.07 (m, 2H).

### Example 337: (R)-4-((1-(4-(2-(2-Aminopyridin-3-yl)-5-phenyl-3H-imidazo[4,5-b]pyridin-3-yl)benzyl)piperidin-3-yl)amino)-1,3,5-triazine-2-carbonitrile

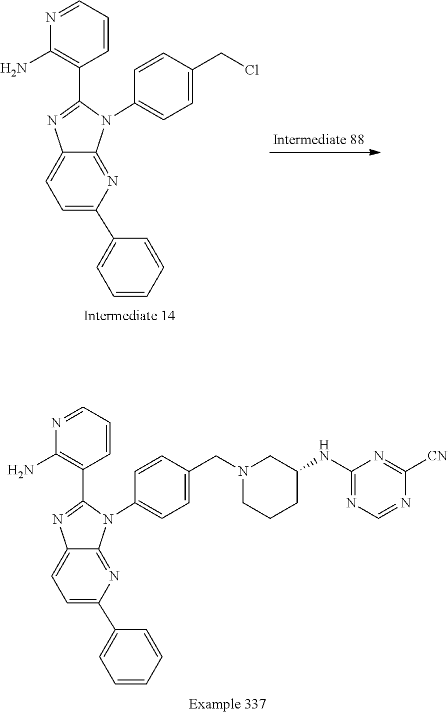 [SMILES: Nc1ncccc1-c1nc2ccc(-c3ccccc3)nc2n1-c1ccc(CCl)cc1 ; N#Cc1ncnc(N[C@@H]2CCCN(Cc3ccc(-n4c(-c5cccnc5N)nc5ccc(-c6ccccc6)nc54)cc3)C2)n1]

To a solution of Intermediate 14 (180 mg, HCl salt, 401 μmol) and Intermediate 88 (127 mg, TFA salt, 401 μmol) in DMF (2 mL) were added NaI (30.1 mg, 200 μmol) and K₂CO₃ (277 mg, 2.01 mmol). The mixture was stirred at 50° C. for 2 hr. The mixture was concentrated under reduced pressure. The residue was purified by prep-HPLC (column: [water (NH₄HCO₃)-ACN]; B %: 50%-80%, 14 min) to give (R)-4-((1-(4-(2-(2-aminopyridin-3-yl)-5-phenyl-3H-imidazo[4,5-b]pyridin-3-yl)benzyl)piperidin-3-yl)amino)-1,3,5-triazine-2-carbonitrile (Example 337, 75.8 mg, yield: 32%) as a yellow solid. MS: m/z=580.4 [M+H]⁺. ¹H NMR (400 MHz, Dimethylsulfoxide-d₆) δ 8.87 (br s, 1H), 8.63 (d, J=16.8 Hz, 1H), 8.27 (d, J=8.4 Hz, 1H), 8.25-7.97 (m, 4H), 7.52-7.38 (m, 7H), 7.13 (d, J=7.6 Hz, 1H), 7.04 (d, J=7.6 Hz, 2H), 6.33-6.27 (m, 1H), 4.08-3.88 (m, 1H), 3.62 (d, J=8.0 Hz, 2H), 2.93-2.86 (m, 1H), 2.75-2.69 (m, 1H), 2.05-1.95 (m, 3H), 1.88-1.82 (m, 1H), 1.76-1.69 (m, 1H), 1.59-1.49 (m, 1H).

### Example 338: 4-((1S,4S)-5-(4-(2-(2-Aminopyridin-3-yl)-5-phenyl-3H-imidazo[4,5-b]pyridin-3-yl)benzyl)-2,5-diazabicyclo[2.2.1]heptan-2-yl)-1,3,5-triazine-2-carbonitrile

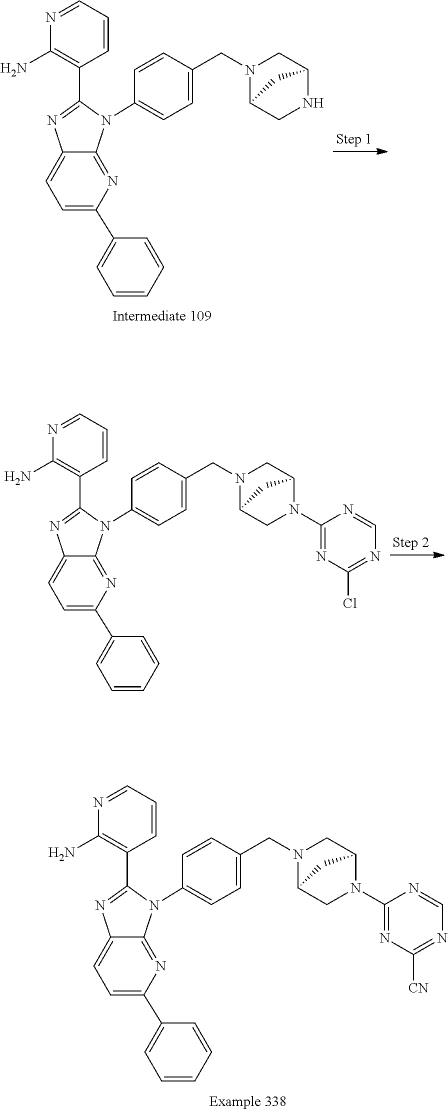 [SMILES: Nc1ncccc1-c1nc2ccc(-c3ccccc3)nc2n1-c1ccc(CN2C[C@@H]3C[C@H]2CN3)cc1 ; Nc1ncccc1-c1nc2ccc(-c3ccccc3)nc2n1-c1ccc(CN2C[C@@H]3C[C@H]2CN3c2ncnc(Cl)n2)cc1 ; N#Cc1ncnc(N2C[C@@H]3C[C@H]2CN3Cc2ccc(-n3c(-c4cccnc4N)nc4ccc(-c5ccccc5)nc43)cc2)n1]

### Step 1: 3-(3-(4-(((1S,4S)-5-(4-Chloro-1,3,5-triazin-2-yl)-2,5-diazabicyclo[2.2.1]heptan-2-yl)methyl)phenyl)-5-phenyl-3H-imidazo[4,5-b]pyridin-2-yl)pyridin-2-amine

To a solution of Intermediate 109 (80.0 mg, 169 μmol) and 2,4-dichloro-1,3,5-triazine (27.8 mg, 186 μmol) in THF (5 mL) and H₂O (1 mL) was added DIEA (109 mg, 845 μmol). The mixture was stirred at 0° C. for 1 hr. The mixture was quenched with water (10 mL) and extracted with EtOAc (10 mL×3). The combined organic layers were dried over anhydrous Na₂SO₄, filtered and concentrated to give 3-(3-(4-(((1S,4S)-5-(4-Chloro-1,3,5-triazin-2-yl)-2,5-diazabicyclo[2.2.1]heptan-2-yl)methyl)phenyl)-5-phenyl-3H-imidazo[4,5-b]pyridin-2-yl)pyridin-2-amine (99.2 mg) as a yellow solid, which was used in the next step without further purification. MS: m/z=587.1 [M+H]⁺.

### Step 2: 4-((1S,4S)-5-(4-(2-(2-Aminopyridin-3-yl)-5-phenyl-3H-imidazo[4,5-b]pyridin-3-yl)benzyl)-2,5-diazabicyclo[2.2.1]heptan-2-yl)-1,3,5-triazine-2-carbonitrile

To a solution of 3-(3-(4-((3-(4-chloro-1,3,5-triazin-2-yl)-3,8-diazabicyclo[3.2.1]octan-8-yl)methyl)phenyl)-5-phenyl-3H-imidazo[4,5-b]pyridin-2-yl)pyridin-2-amine (70 mg, 119 mol) in DMSO (1 mL) were added KCN (15.5 mg, 238 μmol) and DABCO (2.67 mg, 23.8 μmol). The mixture was stirred at 25° C. for 16 hr. The reaction was filtered and concentrated under reduced pressure. The mixture was quenched with H₂O (10 mL) and extracted with EtOAc (10 mL×3). The combined organic layers were dried over anhydrous Na₂SO₄, filtered and concentrated. The residue was purified by prep-TLC (CH₂Cl₂:MeOH=10:1) to give 4-((1S,4S)-5-(4-(2-(2-aminopyridin-3-yl)-5-phenyl-3H-imidazo[4,5-b]pyridin-3-yl)benzyl)-2,5-diazabicyclo[2.2.1]heptan-2-yl)-1,3,5-triazine-2-carbonitrile (Example 338, 3.4 mg, yield: 5%) as a yellow solid. MS: m/z=578.3 [M+H]⁺. ¹H NMR (400 MHz, Chloroform-d) δ 8.54 (d, J=18.4 Hz, 1H), 8.13 (d, J=8.0 Hz, 1H), 8.06 (d, J=4.4 Hz, 1H), 8.01 (d, J=7.2 Hz, 2H), 7.80 (d, J=8.4 Hz, 1H), 7.51-7.38 (m, 7H), 7.10 (dd, J=7.6, 1.6 Hz, 1H), 6.65 (br s, 2H), 6.39-6.35 (m, 1H), 4.97-4.95 (m, 1H), 3.87 (s, 2H), 3.85-3.82 (m, 1H), 3.74-3.72 (m, 1H), 3.49-3.43 (m, 1H), 3.08-3.04 (m, 1H), 2.76-2.68 (m, 1H), 2.21-2.14 (m, 1H), 1.89-1.85 (m, 1H).

### Example 339: 4-((3aR,6aS)-5-(4-(2-(2-aminopyridin-3-yl)-5-phenyl-3H-imidazo[4,5-b]pyridin-3-yl)benzyl)hexahydropyrrolo[3,4-c]pyrrol-2(1H)-yl)pyrimidine-2-carbonitrile

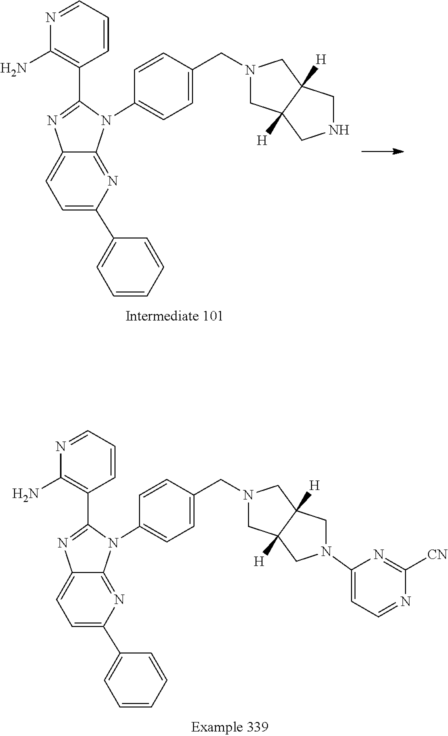 [SMILES: Nc1ncccc1-c1nc2ccc(-c3ccccc3)nc2n1-c1ccc(CN2C[C@H]3CNC[C@H]3C2)cc1 ; N#Cc1nccc(N2C[C@H]3CN(Cc4ccc(-n5c(-c6cccnc6N)nc6ccc(-c7ccccc7)nc65)cc4)C[C@H]3C2)n1]

A mixture of Intermediate 101 (100 mg, 205 μmol), 4-chloropyrimidine-2-carbonitrile (28.6 mg, 205 μmol), and DIEA (79.5 mg, 107.17 μL) in NMP (I mL) was taken up into a microwave tube. The sealed tube was heated at 160° C. for 1 hr under microwave. Water (10 mL) was added to the mixture, and the mixture was extracted with EtOAc (20 mL×2). The combined organic layers were washed with brine (10 mL), dried over anhydrous Na₂SO₄, filtered and concentrated. The residue was purified by silica gel flash chromatography (Eluent of 0˜10% MeOH in CH₂Cl₂) and purified by prep-HPLC (column: Boston Prime C18 150×30 mm×5 μm; mobile phase: [water (NH₃H₂O+NH₄HCO₃)-ACN]; gradient: 66%-96% B over 7 min) to give 4-((3aR,6aS)-5-(4-(2-(2-aminopyridin-3-yl)-5-phenyl-3H-imidazo[4,5-b]pyridin-3-yl)benzyl)hexahydropyrrolo[3,4-c]pyrrol-2(1H)-yl)pyrimidine-2-carbonitrile (Example 339, 8.7 mg, 7.2% yield) as a yellow solid. MS: m/z=591.2 [M+H]⁺. ¹H NMR (400 MHz, Dimethylsulfoxide-d₆) δ 8.38-8.16 (m, 2H), 8.09-7.90 (m, 4H), 7.53-7.33 (m, 7H), 7.15 (d, J=7.6 Hz, 1H), 7.02 (br s, 2H), 6.78 (d, J=5.6 Hz, 1H), 6.37-6.35 (m, 1H), 3.82-3.60 (m, 4H), 2.98-2.96 (m, 2H), 2.65-2.53 (m, 6H).

### Example 340: 4-((3aR,6aS)-5-(4-(2-(2-Aminopyridin-3-yl)-5-phenyl-3H-imidazo[4,5-b]pyridin-3-yl)benzyl)hexahydropyrrolo[3,4-c]pyrrol-2(1H)-yl)-1,3,5-triazine-2-carbonitrile

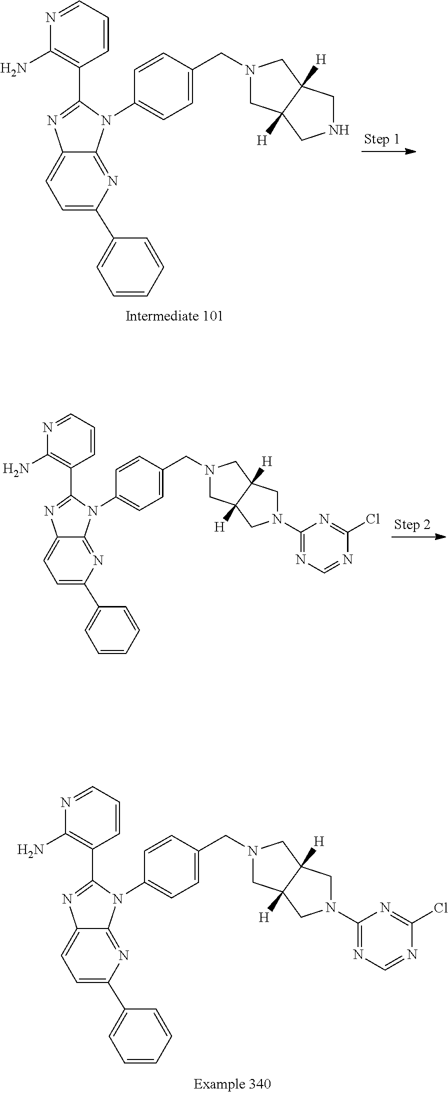 [SMILES: Nc1ncccc1-c1nc2ccc(-c3ccccc3)nc2n1-c1ccc(CN2C[C@H]3CNC[C@H]3C2)cc1 ; Nc1ncccc1-c1nc2ccc(-c3ccccc3)nc2n1-c1ccc(CN2C[C@@H]3CN(c4ncnc(Cl)n4)C[C@@H]3C2)cc1 ; Nc1ncccc1-c1nc2ccc(-c3ccccc3)nc2n1-c1ccc(CN2C[C@@H]3CN(c4ncnc(Cl)n4)C[C@@H]3C2)cc1]

### Step 1: 3-(3-(4-(((3aR,6aS)-5-(4-Chloro-1,3,5-triazin-2-yl)hexahydropyrrolo[3,4-c]pyrrol-2(1H)-yl)methyl)phenyl)-5-phenyl-3H-imidazo[4,5-b]pyridin-2-yl)pyridin-2-amine

To a solution of Intermediate 101 (200 mg, HCl salt, 382 μmol) in THF (4 mL) and H₂O (1 mL) were added DIEA (158 mg, 1.14 mmol) and 2,4-dichloro-1,3,5-triazine (63.0 mg, 420 μmol) at 0° C. This mixture was stirred at 0° C. for 0.5 hr. H₂O (5 mL) was added to the mixture. The aqueous phase was extracted with EtOAc (20 mL×3). The combined organic phases were washed with brine (30 mL), dried over anhydrous Na₂SO₄, filtered and concentrated. The residue was purified by prep-TLC (EtOAc:petroleum ether=1:0) to give 3-(3-(4-(((3aR,6aS)-5-(4-chloro-1,3,5-triazin-2-yl)hexahydropyrrolo[3,4-c]pyrrol-2(1H)-yl)methyl)phenyl)-5-phenyl-3H-imidazo[4,5-b]pyridin-2-yl)pyridin-2-amine (110 mg, yield: 48%) as a light-yellow solid. MS: m/z=601.4 [M+H]⁺.

### Step 2: 4-((3aR,6aS)-5-(4-(2-(2-Aminopyridin-3-yl)-5-phenyl-3H-imidazo[4,5-b]pyridin-3-yl)benzyl)hexahydropyrrolo[3,4-c]pyrrol-2(1H)-yl)-1,3,5-triazine-2-carbonitrile

To a solution of 3-(3-(4-(((3aR,6aS)-5-(4-chloro-1,3,5-triazin-2-yl)hexahydropyrrolo[3,4-c]pyrrol-2(1H)-yl)methyl)phenyl)-5-phenyl-3H-imidazo[4,5-b]pyridin-2-yl)pyridin-2-amine (50.0 mg, 83.2 μmol) in DMSO (2 mL) was added KCN (170 mg, 2.61 mmol) and DABCO (2 mg, 16.6 μmol) at 25° C. This mixture was stirred at 25° C. for 16 hr. H₂O (5 mL) was added to the mixture. The aqueous phase was extracted with EtOAc (20 mL×3). The combined organic layers were washed with brine (30 mL), dried over anhydrous Na₂SO₄, filtered and concentrated. The residue was purified by prep-TLC (CH₂Cl₂:MeOH=30:1) to give 4-((3aR,6aS)-5-(4-(2-(2-aminopyridin-3-yl)-5-phenyl-3H-imidazo[4,5-b]pyridin-3-yl)benzyl)hexahydropyrrolo[3,4-c]pyrrol-2(1H)-yl)-1,3,5-triazine-2-carbonitrile (Example 340, 5.50 mg, yield: 11%) as an off-white solid. MS: m/z=592.4 [M+H]⁺. ¹H NMR (400 MHz, Methanol-d₄) δ 8.54 (s, 1H), 8.19 (d, J=8.8 Hz, 1H), 8.03 (d, J=7.6 Hz, 2H), 7.98 (d, J=4.8 Hz, 1H), 7.94 (d, J=8.0 Hz, 1H), 7.50 (d, J=8.0 Hz, 2H), 7.46-7.35 (m, 5H), 7.32 (d, J=7.6 Hz, 1H), 6.48 (dd, J=7.6, 5.2 Hz, 1H), 3.91-3.81 (m, 2H), 3.75-3.58 (m, 4H), 3.08-3.02 (m, 2H), 2.80-2.72 (m, 2H), 2.67-2.58 (m, 2H).

### Example 341: 4-((3aR,6aR)-5-(4-(2-(2-Aminopyridin-3-yl)-5-phenyl-3H-imidazo[4,5-b]pyridin-3-yl)benzyl)hexahydropyrrolo[3,4-c]pyrrol-2(1H)-yl)pyrimidine-2-carbonitrile

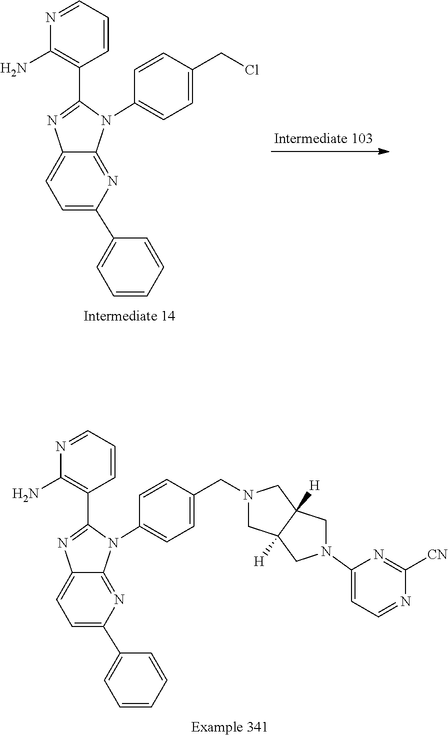 [SMILES: Nc1ncccc1-c1nc2ccc(-c3ccccc3)nc2n1-c1ccc(CCl)cc1 ; N#Cc1nccc(N2C[C@H]3CN(Cc4ccc(-n5c(-c6cccnc6N)nc6ccc(-c7ccccc7)nc65)cc4)C[C@@H]3C2)n1]

To a mixture of Intermediate 103 (87.9 mg, TFA salt, 267 μmol) and Intermediate 14 (100 mg, 243 μmol) in DMF (1 mL) was added DIEA (126 mg, 169 μL). The mixture was stirred at 80° C. for 12 hr. The mixture was quenched with H₂O (50 mL) and extracted with EtOAc (30 mL×2). The combined organic layers were washed with brine (20 mL), dried over Na₂SO₄, filtered and concentrated. The mixture was purified by prep-HPLC (column: Phenomenex C18 80*40 mm*3 um; mobile phase: [water (NH₃H₂O+NH₄HCO₃)-ACN]; gradient: 62%-92% B over 7 min) to give 4-((3aR,6aR)-5-(4-(2-(2-aminopyridin-3-yl)-5-phenyl-3H-imidazo[4,5-b]pyridin-3-yl)benzyl)hexahydropyrrolo[3,4-c]pyrrol-2(1H)-yl)pyrimidine-2-carbonitrile (Example 341, 25.4 mg, yield: 18%) as a yellow solid. MS: m/z=591.4 [M+H]⁺. ¹H NMR (400 MHz, Dimethylsulfoxide-d₆) 8.32-8.15 (m, 2H), 8.05-7.97 (m, 4H), 7.53-7.43 (m, 6H), 7.42-7.37 (m, 1H), 7.17 (dd, J=7.6, 1.6 Hz, 1H), 7.02 (br s, 2H), 6.74 (d, J=6.4 Hz, 1H), 6.43-6.34 (m, 1H), 4.04-3.91 (m, 2H), 3.86-3.78 (m, 1H), 3.65-3.57 (m, 1H), 3.16-3.06 (m, 2H), 2.96-2.86 (m, 2H), 2.73-2.62 (m, 2H), 2.44-2.26 (m, 2H).

### Example 342: 4-(4-(4-(2-(2-Aminopyridin-3-yl)-5-(4-chlorophenyl)-3H-imidazo[4,5-b]pyridin-3-yl)benzyl)piperazin-1-yl)pyrimidine-2-carbonitrile

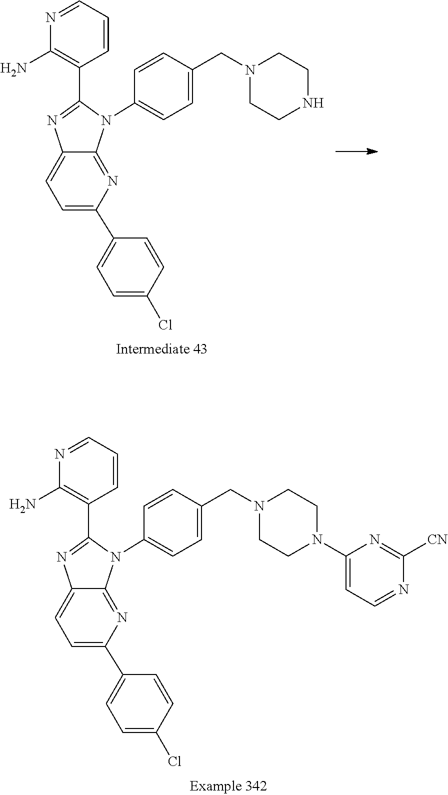 [SMILES: Nc1ncccc1-c1nc2ccc(-c3ccc(Cl)cc3)nc2n1-c1ccc(CN2CCNCC2)cc1 ; N#Cc1nccc(N2CCN(Cc3ccc(-n4c(-c5cccnc5N)nc5ccc(-c6ccc(Cl)cc6)nc54)cc3)CC2)n1]

A mixture of Intermediate 43 (100 mg, 202 μmol), 4-chloropyrimidine-2-carbonitrile (33.8 mg, 242 μmol) and DIEA (104 mg, 806 μmol) in NMP (2 mL) was taken up into a microwave tube. The sealed tube was heated at 130° C. for 1 hr under microwave. The reaction mixture was quenched with H₂O (5 mL) at 0° C., and then diluted with CH₂Cl₂ (10 mL) and extracted with CH₂Cl₂ (15 mL×2). The combined organic layers were washed with brine (15 mL), dried over Na₂SO₄, filtered and concentrated under reduced pressure. After purified by prep-HPLC (column: Phenomenex C18 150*25 mm*10 μm; mobile phase: [water (NH₄HCO₃)-ACN]; B %: 54%-84%, over 14 min), 4-(4-(4-(2-(2-aminopyridin-3-yl)-5-(4-chlorophenyl)-3H-imidazo[4,5-b]pyridin-3-yl)benzyl)piperazin-1-yl)pyrimidine-2-carbonitrile (Example 342, 27.1 mg, yield: 22%) was obtained as a purple lyophilized powder. MS: m/z=599.4 [M+H]⁺. ¹H NMR (400 MHz, Dimethylsulfoxide-d₆) δ 8.30-8.26 (m, 2H), 8.08-7.99 (m, 4H), 7.55-7.50 (m, 4H), 7.49-7.45 (m, 2H), 7.17-7.10 (m, 2H), 7.03 (br s, 2H), 6.36 (dd, J=8.0, 4.8 Hz, 1H), 3.81-3.66 (m, 4H), 3.65 (s, 2H), 2.49-2.46 (m, 4H).

### Example 343: 2-(4-(4-(2-(2-Aminopyridin-3-yl)-5-(4-chlorophenyl)-3H-imidazo[4,5-b]pyridin-3-yl)benzyl)piperazin-1-yl)pyrimidine-4-carbonitrile

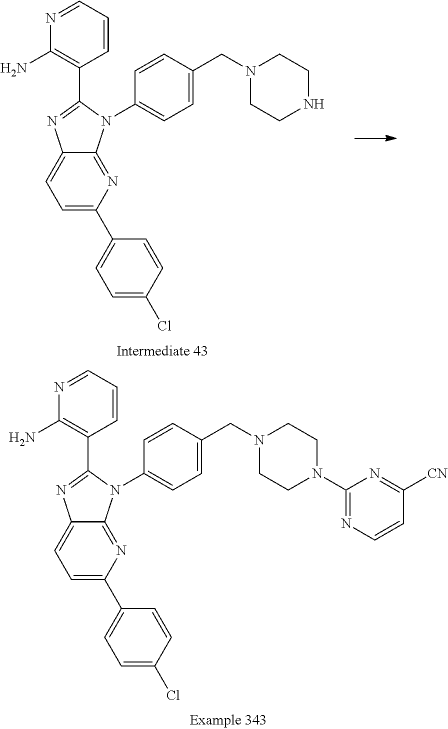 [SMILES: Nc1ncccc1-c1nc2ccc(-c3ccc(Cl)cc3)nc2n1-c1ccc(CN2CCNCC2)cc1 ; N#Cc1ccnc(N2CCN(Cc3ccc(-n4c(-c5cccnc5N)nc5ccc(-c6ccc(Cl)cc6)nc54)cc3)CC2)n1]

A mixture of Intermediate 43 (150 mg, 302 μmol), 2-chloropyrimidine-4-carbonitrile (50.6 mg, 363 μmol) and DIEA (156 mg, 1.21 mmol) in NMP (2 mL) was taken up into a microwave tube. The sealed tube was heated at 130° C. for 1 hr under microwave. The reaction mixture was quenched with H₂O (5 mL) at 0° C., and then diluted with CH₂Cl₂ (10 mL) and extracted with CH₂Cl₂ (15 mL×2). The combined organic layers were washed with brine (10 mL), dried over Na₂SO₄, filtered and concentrated under reduced pressure. After purified by prep-HPLC (column: Phenomenex C18 150*25 mm*10 μm; mobile phase: [water (NH₄HCO₃)-ACN]; B %: 55%-85%, over 10 min), 2-(4-(4-(2-(2-aminopyridin-3-yl)-5-(4-chlorophenyl)-3H-imidazo[4,5-b]pyridin-3-yl)benzyl)piperazin-1-yl)pyrimidine-4-carbonitrile (Example 343, 25.3 mg, yield: 14%) was obtained as a brown lyophilized powder. MS: m/z=599.2 [M+H]⁺. ¹H NMR (400 MHz, Dimethylsulfoxide-d₆) δ 8.65 (d, J=4.8 Hz, 1H), 8.28 (d, J=8.4 Hz, 1H), 8.08-7.99 (m, 4H), 7.55-7.50 (m, 4H), 7.49-7.44 (m, 2H), 7.18-7.12 (m, 2H), 7.03 (br s, 2H), 6.37 (dd, J=8.0, 4.8 Hz, 1H), 3.82-3.77 (m, 4H), 3.64 (s, 2H), 2.61-2.54 (m, 4H).

### Example 344: 6-(4-(4-(2-(2-Aminopyridin-3-yl)-5-(4-chlorophenyl)-3H-imidazo[4,5-b]pyridin-3-yl)benzyl)piperazin-1-yl)pyrimidine-4-carbonitrile

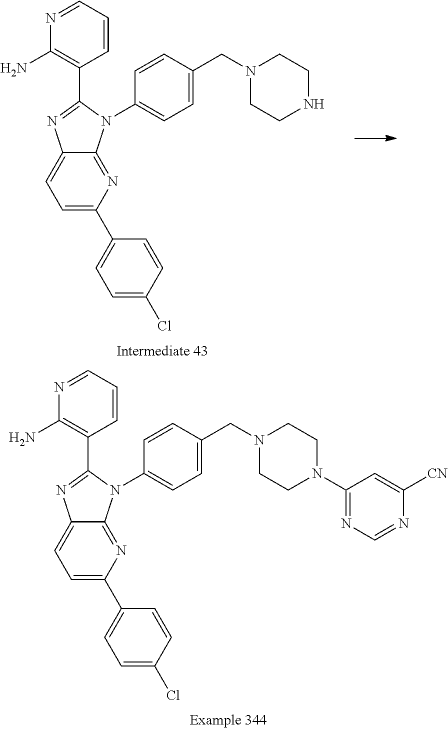 [SMILES: Nc1ncccc1-c1nc2ccc(-c3ccc(Cl)cc3)nc2n1-c1ccc(CN2CCNCC2)cc1 ; N#Cc1cc(N2CCN(Cc3ccc(-n4c(-c5cccnc5N)nc5ccc(-c6ccc(Cl)cc6)nc54)cc3)CC2)ncn1]

A mixture of Intermediate 43 (150 mg, 302 μmol), 6-chloropyrimidine-4-carbonitrile (50.6 mg, 363 μmol) and DIEA (156 mg, 1.21 mmol) in NMP (2 mL) was taken up into a microwave tube. The sealed tube was heated at 130° C. for 1 hr under microwave. The reaction mixture was quenched with H₂O (5 mL) at 0° C., and then diluted with CH₂Cl₂ (10 mL) and extracted with CH₂Cl₂ (15 mL×2). The combined organic layers were washed with brine (15 mL), dried over Na₂SO₄, filtered and concentrated under reduced pressure. After purified by prep-HPLC (column: Phenomenex C18 150*25 mm*5 μm; mobile phase: [water (NH₄HCO₃)-ACN]; B %: 52%-82%, over 10 min), 6-(4-(4-(2-(2-aminopyridin-3-yl)-5-(4-chlorophenyl)-3H-imidazo[4,5-b]pyridin-3-yl)benzyl)piperazin-1-yl)pyrimidine-4-carbonitrile (Example 344, 29.5 mg, yield: 16%) was obtained as a brown lyophilized powder. MS: m/z=599.2 [M+H]⁺. ¹H NMR (400 MHz, Dimethylsulfoxide-d₆) δ 8.57 (s, 1H), 8.28 (d, J=8.4 Hz, 1H), 8.09-7.99 (m, 4H), 7.58-7.45 (m, 7H), 7.14 (dd, J=7.6, 1.6 Hz, 1H), 7.03 (br s, 2H), 6.35 (dd, J=7.6, 4.8 Hz, 1H), 3.87-3.66 (m, 4H), 3.64 (s, 2H), 2.49-2.44 (m, 4H).

### Example 345: 4-(4-(4-(2-(2-Aminopyridin-3-yl)-5-(4-methyl-5-oxo-1,4-diazepan-1-yl)-3H-imidazo[4,5-b]pyridin-3-yl)benzyl)piperazin-1-yl)pyrimidine-2-carbonitrile

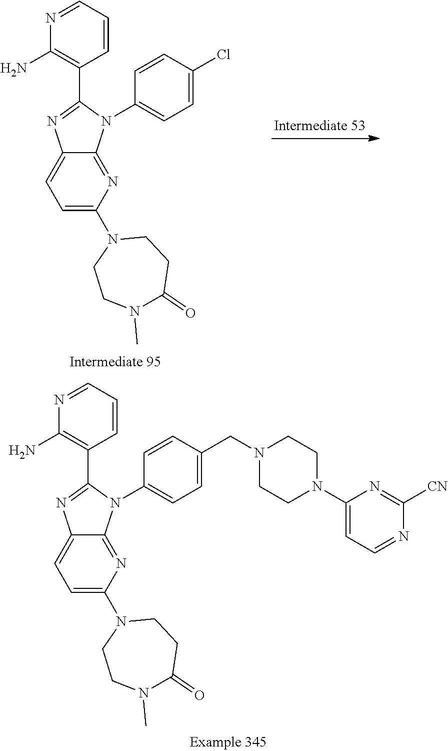 [SMILES: CN1CCN(c2ccc3nc(-c4cccnc4N)n(-c4ccc(Cl)cc4)c3n2)CCC1=O ; CN1CCN(c2ccc3nc(-c4cccnc4N)n(-c4ccc(CN5CCN(c6ccnc(C#N)n6)CC5)cc4)c3n2)CCC1=O]

To a solution of Intermediate 95 (85.4 mg, 185 μmol) in DMF (1 mL) were added DIEA (71.7 mg, 96.6 μL) and Intermediate 53 (38.5 mg, 203 μmol). The mixture was stirred at 80° C. for 2 hr. The mixture was filtered and concentrated under reduced pressure. After purified by prep-HPLC (column: Phenomenex C18 75×30 mm×3 um; mobile phase: [water (NH₃H₂O+NH₄HCO₃)-ACN]; gradient: 38%-68% B over 7 min), 4-(4-(4-(2-(2-aminopyridin-3-yl)-5-(4-methyl-5-oxo-1,4-diazepan-1-yl)-3H-imidazo[4,5-b]pyridin-3-yl)benzyl)piperazin-1-yl)pyrimidine-2-carbonitrile (Example 345, 10.3 mg, yield: 9.1%) was obtained as a light yellow solid. MS: m/z=615.3 [M+H]⁺. ¹H NMR (400 MHz, Dimethylsulfoxide-d₆) δ 8.27 (d, J=6.4 Hz, 1H), 7.99-7.91 (m, 2H), 7.46 (d, J=8.4 Hz, 2H), 7.33 (d, J=8.4 Hz, 2H), 7.12 (d, J=6.4 Hz, 1H), 7.05-6.98 (m, 3H), 6.90 (d, J=8.8 Hz, 1H), 6.31 (dd, J=7.6, 4.4 Hz, 1H), 3.78-3.65 (m, 9H), 3.62 (s, 2H), 3.52-3.40 (m, 2H), 2.82 (s, 3H), 2.62-2.57 (m, 5H).

### Example 346: 4-((1-(4-(2-(2-Aminopyridin-3-yl)-5-(4-methyl-5-oxo-1,4-diazepan-1-yl)-3H-imidazo[4,5-b]pyridin-3-yl)benzyl)piperidin-4-yl)amino)pyrimidine-2-carbonitrile

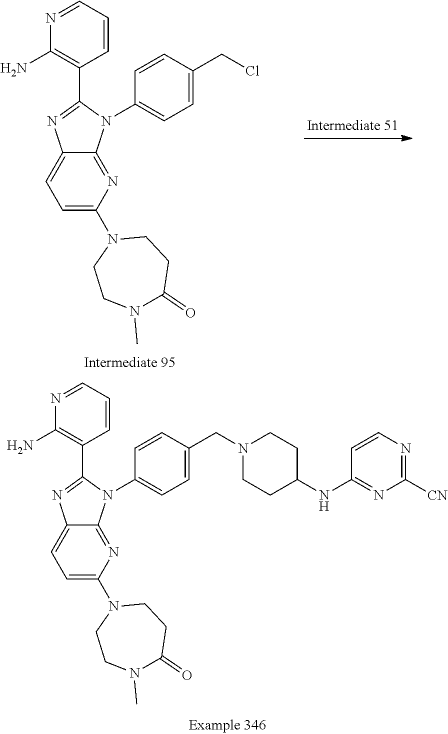 [SMILES: CN1CCN(c2ccc3nc(-c4cccnc4N)n(-c4ccc(CCl)cc4)c3n2)CCC1=O ; CN1CCN(c2ccc3nc(-c4cccnc4N)n(-c4ccc(CN5CCC(Nc6ccnc(C#N)n6)CC5)cc4)c3n2)CCC1=O]

To a solution of Intermediate 95 (100 mg, 217 μmol) in DMF (2 mL) were added DIEA (83.9 mg, 113 μL) and Intermediate 51 (52.8 mg, 260 μmol). The mixture was stirred at 80° C. for 2 hr. The reaction was concentrated. Water (2 mL) was added and the aqueous was extracted with CH₂Cl₂ (2 mL×2). The combined organic layers were dried over anhydrous Na₂SO₄, filtered and concentrated. The residue was purified by prep-HPLC (column: Phenomenex C18 75×30 mm×3 μm; mobile phase: [water (NH₃H₂O+NH₄HCO₃)-ACN]; gradient: 40%-70% B over 7 min) to give 4-((1-(4-(2-(2-aminopyridin-3-yl)-5-(4-methyl-5-oxo-1,4-diazepan-1-yl)-3H-imidazo[4,5-b]pyridin-3-yl)benzyl)piperidin-4-yl)amino)pyrimidine-2-carbonitrile (Example 346, 9.4 mg, yield: 6.9%) as a light-yellow solid. MS: m/z=629.4 [M+H]⁺. ¹H NMR (400 MHz, Dimethylsulfoxide-d₆) δ 8.12-8.03 (m, 2H), 7.98-7.92 (m, 2H), 7.43 (d, J=8.0 Hz, 2H), 7.32 (d, J=8.0 Hz, 2H), 7.05-6.98 (m, 3H), 6.89 (d, J=8.8 Hz, 1H), 6.67 (d, J=6.0 Hz, 1H), 6.32 (dd, J=7.6, 4.8 Hz, 1H), 3.81-3.66 (m, 5H), 3.57 (s, 2H), 3.48-3.44 (m, 2H), 2.81-2.79 (m, 5H), 2.63-2.57 (m, 2H), 2.20-2.09 (m, 2H), 1.94-1.83 (m, 2H), 1.53-1.44 (m, 2H).

### Example 347: 2-((1-(4-(2-(2-Aminopyridin-3-yl)-5-(3-oxo-1,4-diazepan-1-yl)-³H-imidazo[4,5-b]pyridin-3-yl)benzyl)piperidin-4-yl)amino)pyrimidine-4-carbonitrile

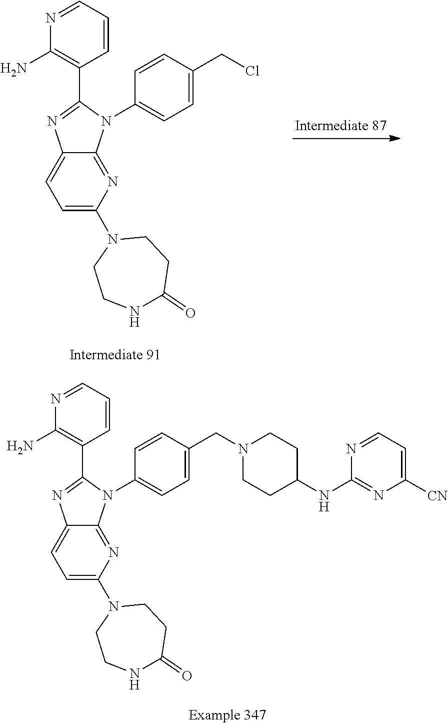 [SMILES: Nc1ncccc1-c1nc2ccc(N3CCNC(=O)CC3)nc2n1-c1ccc(CCl)cc1 ; N#Cc1ccnc(NC2CCN(Cc3ccc(-n4c(-c5cccnc5N)nc5ccc(N6CCNC(=O)CC6)nc54)cc3)CC2)n1]

To a solution of Intermediate 87 (105 mg, 516 μmol) and Intermediate 91 (210 mg, 469 mol) in NMP (0.5 mL) were added K₂CO₃ (194 mg, 1.41 mmol) and NaI (14.1 mg, 93.8 μmol). The mixture was stirred at 80° C. for 1 hr. The crude product was concentrated. The filtrate was purified by prep-HPLC column: Phenomenex C18 75×30 mm×3 μm; mobile phase: [water (NH₃H₂O+NH₄HCO₃)-ACN]; gradient: 35%-75%, B over 7 min) to give 2-((I-(4-(2-(2-Aminopyridin-3-yl)-5-(3-oxo-1,4-diazepan-1-yl)-3H-imidazo[4,5-b]pyridin-3-yl)benzyl)piperidin-4-yl)amino)pyrimidine-4-carbonitrile (Example 347, 21.9 mg, yield: 7.6%) as a light-yellow solid. MS: m/z=615.5 [M+H]⁺. ¹H NMR (400 MHz, Dimethylsulfoxide-d₆) δ 8.54 (br s, 1H), 8.02-7.89 (m, 2H), 7.84 (d, J=7.2 Hz, 1H), 7.50-7.27 (m, 5H), 7.12-6.92 (m, 4H), 6.80 (d, J=8.8 Hz, 1H), 6.32 (dd, J=7.2, 5.2 Hz 1H), 4.17 (s, 2H), 3.82-3.81 (m, 2H), 3.76-3.63 (m, 1H), 3.55 (s, 2H), 3.18-3.17 (m, 2H), 2.91-2.77 (m, 2H), 2.00-2.16 (m, 2H), 1.87-1.79 (m, 2H), 1.65-1.64 (m, 2H), 1.61-1.43 (m, 2H).

### Example 348: 4-(4-(4-(2-(2-Aminopyridin-3-yl)-5-(1-methyl-6-oxo-1,6-dihydropyridin-3-yl)-3H-imidazo[4,5-b]pyridin-3-yl)benzyl)piperazin-1-yl)pyrimidine-2-carbonitrile

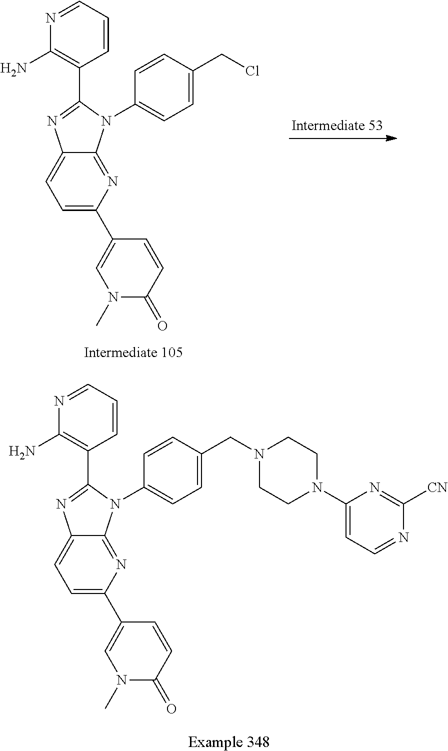 [SMILES: Cn1cc(-c2ccc3nc(-c4cccnc4N)n(-c4ccc(CCl)cc4)c3n2)ccc1=O ; Cn1cc(-c2ccc3nc(-c4cccnc4N)n(-c4ccc(CN5CCN(c6ccnc(C#N)n6)CC5)cc4)c3n2)ccc1=O]

To a mixture of Intermediate 105 (80 mg, 181 μmol) and Intermediate 53 (37.6 mg, 199 μmol) in DMF (1 mL) was added DIEA (93.4 mg, 126 μL). The mixture was stirred at 80° C. for 1 hr. The mixture was concentrated and purified by prep-HPLC (column: Phenomenex C18 75*30 mm*3 μm; mobile phase: [water (NH₃H₂O+NH₄HCO₃)-ACN]; gradient: 28%-58% B over 7 min) to give 4-(4-(4-(2-(2-aminopyridin-3-yl)-5-(1-methyl-6-oxo-1,6-dihydropyridin-3-yl)-3H-imidazo[4,5-b]pyridin-3-yl)benzyl)piperazin-1-yl)pyrimidine-2-carbonitrile (Example 348, 54.3 mg, yield: 51%) as a yellow solid. MS: m/z=596.1 [M+H]⁺. ¹H NMR (400 MHz, Dimethylsulfoxide-d₆) δ 8.48 (d, J=2.8 Hz, 1H), 8.30-8.20 (m, 2H), 8.08 (dd, J=9.6, 2.8 Hz, 1H), 7.99 (dd, J=4.8, 2.0 Hz, 1H), 7.84 (d, J=8.8 Hz, 1H), 7.53-7.47 (m, 2H), 7.46-7.38 (m, 2H), 7.16-7.09 (m, 2H), 7.03 (br s, 2H), 6.49 (d, J=9.6 Hz, 1H), 6.35 (dd, J=7.6, 4.8 Hz, 1H), 3.82-3.65 (m, 4H), 3.64 (s, 2H), 3.52 (s, 3H), 3.35-3.33 (m, 4H).

### Example 349: 4-(4-(4-(2-(2-Aminopyridin-3-yl)-5-(1-methyl-2-oxo-1,2-dihydropyridin-4-yl)-3H-imidazo[4,5-h]pyridin-3-yl)benzyl)piperazin-1-yl)pyrimidine-2-carbonitrile

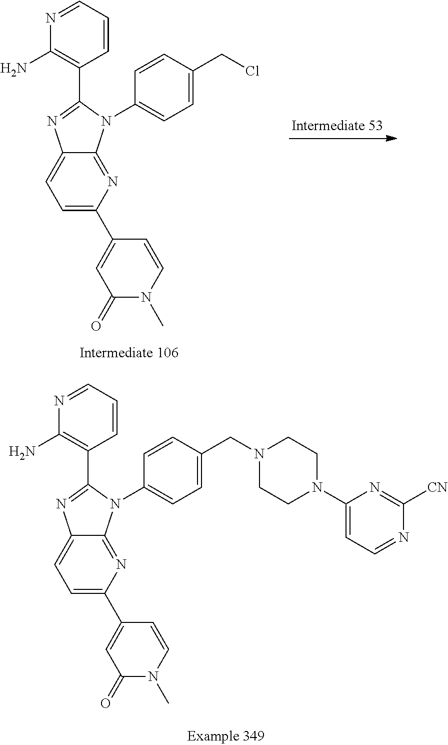 [SMILES: Cn1ccc(-c2ccc3nc(-c4cccnc4N)n(-c4ccc(CCl)cc4)c3n2)cc1=O ; Cn1ccc(-c2ccc3nc(-c4cccnc4N)n(-c4ccc(CN5CCN(c6ccnc(C#N)n6)CC5)cc4)c3n2)cc1=O]

To a mixture of Intermediate 106 (100 mg, 226 μmol) and Intermediate 53 (47.0 mg, 248 μmol) in DMF (1 mL) was added DIEA (117 mg, 157 μL). The mixture was stirred at 80° C. for 1 hr. The mixture was concentrated. After purified by prep-HPLC (column: Phenomenex C18 75*30 mm*3 μm; mobile phase: [water (NH₃H₂O+NH₄CO₃)-ACN]; gradient: 30%-60% B over 7 min), 4-(4-(4-(2-(2-aminopyridin-3-yl)-5-(1-methyl-2-oxo-1,2-dihydropyridin-4-yl)-3H-imidazo[4,5-b]pyridin-3-yl)benzyl)piperazin-1-yl)pyrimidine-2-carbonitrile (Example 349, 89.1 mg, yield: 66%) was obtained as a yellow solid. MS: m/z=596.2 [M+H]^{+ 1}H NMR (400 MHz, Dimethylsulfoxide-d₆) δ 8.33-8.24 (m, 2H), 8.05 (d, J=8.4 Hz, 1H), 8.01 (dd, J=4.8, 2.0 Hz, 1H), 7.76 (d, J=7.2 Hz, 1H), 7.55-7.49 (m, 2H), 7.48-7.43 (m, 2H), 7.17 (dd, J=7.6, 1.6 Hz, 1H), 7.11 (d, J=6.8 Hz, 1H), 7.08-6.98 (m, 3H), 6.86 (dd, 1=7.2, 2.0 Hz, 1H), 6.37 (dd, 1=7.6, 4.4 Hz, 1H), 3.93-3.66 (m, 4H), 3.64 (s, 2H), 3.44 (s, 3H), 3.35-3.32 (m, 4H).

### Example 350: 2-(4-(4-(2-(2-Aminopyridin-3-yl)-5-cyclopropyl-3H-imidazo[4,5-b]pyridin-3-yl)benzyl)piperazin-1-yl)pyrimidine-4-carbonitrile

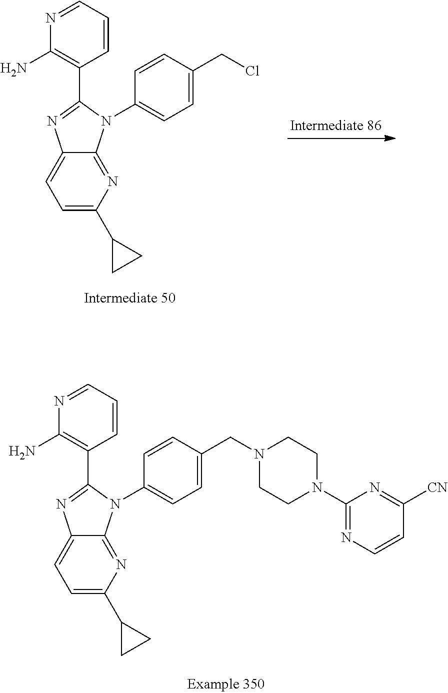 [SMILES: Nc1ncccc1-c1nc2ccc(C3CC3)nc2n1-c1ccc(CCl)cc1 ; N#Cc1ccnc(N2CCN(Cc3ccc(-n4c(-c5cccnc5N)nc5ccc(C6CC6)nc54)cc3)CC2)n1]

To a solution of Intermediate 50 (200 mg, 485 μmol) and Intermediate 86 (147 mg, 485 μmol) in DMF (2 mL) were added NaI (36.4 mg, 242 μmol) and K₂CO₃ (335 mg, 2.43 mmol). The mixture was stirred at 50° C. for 1 hr. The reaction mixture was filtered. The residue was purified by prep-HPLC (column: [water (NH₄HCO₃)-ACN]; B %: 51%-81%, 14 min), 2-(4-(4-(2-(2-aminopyridin-3-yl)-5-cyclopropyl-3H-imidazo[4,5-b]pyridin-3-yl)benzyl)piperazin-1-yl)pyrimidine-4-carbonitrile (Example 350, 85.2 mg, yield: 33%) was obtained as a yellow solid. MS: m/z=529.4 [M+H]⁺. ¹H NMR (400 MHz, Dimethylsulfoxide-d₆) δ 8.64 (d, J=4.8 Hz, 1H), 8.04 (d, J=8.0 Hz, 1H), 7.96 (dd, J=4.4, 1.6 Hz, 1H), 7.47 (d, J=8.4 Hz, 2H), 7.34 (d, J=8.0 Hz, 2H), 7.24 (d, J=8.4 Hz, 1H), 7.16 (d, J=4.4 Hz, 1H), 7.06 (dd, J=7.6, 2.0 Hz, 1H), 6.97 (br s, 2H), 6.34 (dd, J=8.0, 4.8 Hz, 1H), 3.82-3.74 (m, 4H), 3.62 (s, 2H), 2.49-2.45 (m, 4H), 2.21-2.12 (m, 1H), 0.97-0.90 (m, 2H), 0.86-0.80 (m, 2H).

### Example 351: 2-((1-(4-(2-(2-Aminopyridin-3-yl)-5-cyclopropyl-3H-imidazo[4,5-b]pyridin-3-yl)benzyl)piperidin-4-yl)amino)pyrimidine-4-carbonitrile

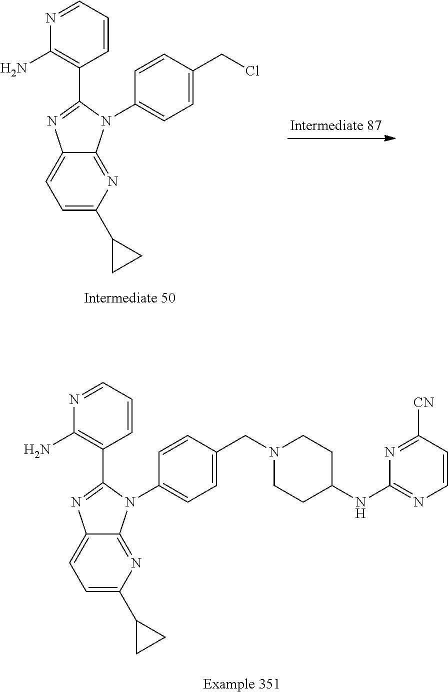 [SMILES: Nc1ncccc1-c1nc2ccc(C3CC3)nc2n1-c1ccc(CCl)cc1 ; N#Cc1ccnc(NC2CCN(Cc3ccc(-n4c(-c5cccnc5N)nc5ccc(C6CC6)nc54)cc3)CC2)n1]

To a solution of Intermediate 50 (200 mg, 485 μmol) and Intermediate 87 (153 mg, 485 μmol) in DMF (2 mL) were added NaI (36.3 mg, 242 μmol) and K₂CO₃ (335 mg, 2.43 mmol). The mixture was stirred at 50° C. for 1 hr. The reaction mixture was filtered. The residue was purified by prep-HPLC (column: [water (NH₄HCO₃)-ACN]; B %: 45%-75%, 14 min) to give 2-((1-(4-(2-(2-aminopyridin-3-yl)-5-cyclopropyl-3H-imidazo[4,5-b]pyridin-3-yl)benzyl)piperidin-4-yl)amino)pyrimidine-4-carbonitrile (Example 351, 15.9 mg, yield: 6%) as a yellow solid. MS: m/z=543.4 [M+H]⁺. ¹H NMR (400 MHz, Dimethylsulfoxide-d₆) δ 8.62 (d, J=4.8 Hz, 1H), 8.03 (d, J=8.0 Hz, 1H), 7.99-7.94 (m, 1H), 7.49 (d, J=8.0 Hz, 2H), 7.31 (d, J=8.0 Hz, 2H), 7.25 (d, J=8.4 Hz, 1H), 7.13-7.07 (m, 2H), 6.95 (br s, 2H), 6.36 (dd, J=7.6, 4.8 Hz, 1H), 4.48-4.32 (m, 2H), 3.84 (s, 2H), 3.18-3.10 (m, 2H), 2.80-2.69 (m, 1H), 2.32-2.22 (m, 1H), 2.22-2.11 (m, 1H), 1.95-1.87 (m, 2H), 1.33-1.23 (m, 2H), 0.95-0.89 (m, 2H), 0.84-0.79 (m, 2H).

### Example 352: 2-((1-(4-(2-(2-Aminopyridin-3-yl)-5-(4-fluorophenyl)-3H-imidazo[4,5-b]pyridin-3-yl)benzyl)piperidin-4-yl)amino)pyrimidine-4-carbonitrile

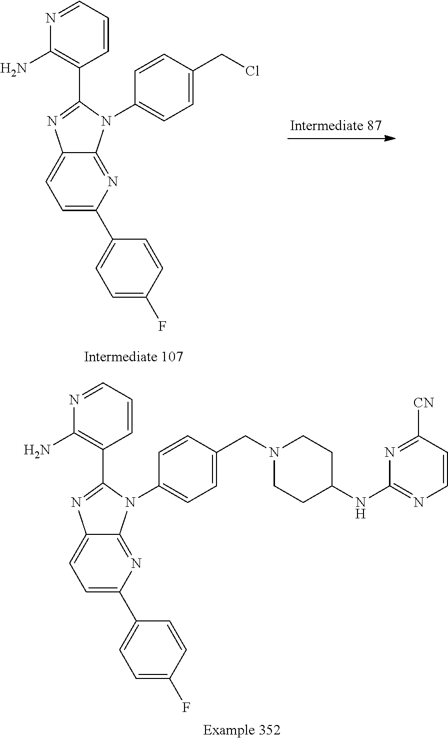 [SMILES: Nc1ncccc1-c1nc2ccc(-c3ccc(F)cc3)nc2n1-c1ccc(CCl)cc1 ; N#Cc1ccnc(NC2CCN(Cc3ccc(-n4c(-c5cccnc5N)nc5ccc(-c6ccc(F)cc6)nc54)cc3)CC2)n1]

To a solution of Intermediate 107 (200 mg, 429 μmol) and Intermediate 87 (136 mg, 429 μmol) in DMF (2 mL) were added NaI (32.3 mg, 214 μmol) and K₂CO₃ (296 mg, 2.14 mmol). The mixture was stirred at 50° C. for 1 hr. The reaction mixture was filtered. The residue was purified by prep-HPLC (column: [water (NH₄HCO₃)-ACN]; B %: 53%-83%, 14 min) to give 2-((1-(4-(2-(2-aminopyridin-3-yl)-5-(4-fluorophenyl)-3H-imidazo[4,5-b]pyridin-3-yl)benzyl)piperidin-4-yl)amino)pyrimidine-4-carbonitrile (Example 352, 72.1 mg, yield: 28%) as a yellow solid. MS: m/z=597.4 [M+H]⁺. ¹H NMR (400 MHz, Dimethylsulfoxide-d₆) δ 8.62 (d, J=4.4 Hz, 1H), 8.26 (d, J=8.4 Hz, 1H), 8.11-8.04 (m, 2H), 8.02-7.94 (m, 2H), 7.54 (d, J=8.0 Hz, 2H), 7.43 (d, J=8.0 Hz, 2H), 7.34-7.26 (m, 2H), 7.19 (dd, J=7.6, 2.0 Hz, 1H), 7.10 (d, J=4.8 Hz, J H), 7.02 (br s, 2H), 6.40 (dd, J=8.0, 5.2 Hz, 1H), 4.44-4.38 (m, 2H), 3.86 (s, 2H), 3.20-3.09 (m, 2H), 2.81-2.71 (m, 1H), 2.45-2.19 (m, 1H), 1.95-1.89 (m, 2H), 1.34-1.21 (m, 2H). ¹⁹H NMR (400 MHz, Dimethylsulfoxide-d₆) δ −113.573.

### Example 353: 4-((1-(4-(2-(2-Aminopyridin-3-yl)-5-(3-chlorophenyl)-3H-imidazo[4,5-b]pyridin-3-yl)benzyl)piperidin-4-yl)amino)pyrimidine-2-carbonitrile

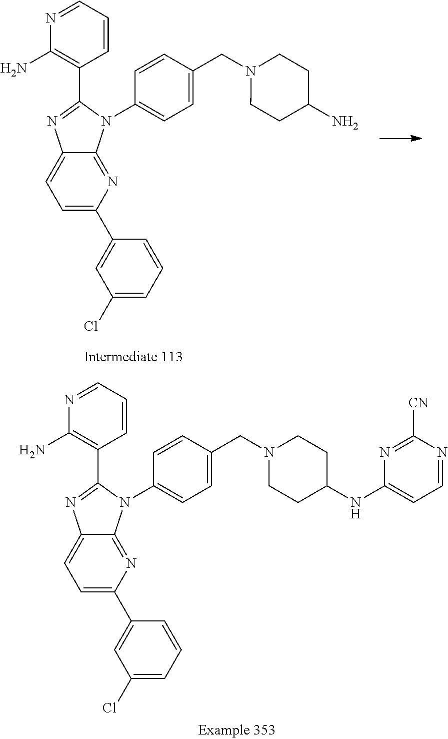 [SMILES: Nc1ncccc1-c1nc2ccc(-c3cccc(Cl)c3)nc2n1-c1ccc(CN2CCC(N)CC2)cc1 ; N#Cc1nccc(NC2CCN(Cc3ccc(-n4c(-c5cccnc5N)nc5ccc(-c6cccc(Cl)c6)nc54)cc3)CC2)n1]

To a solution of Intermediate 113 (100 mg, 196 μmol), 4-chloropyrimidine-2-carbonitrile (30.1 mg, 216 μmol) and DIEA (76 mg, 588 μmol) in NMP (2 mL) was taken up into a microwave tube. The mixture was stirred at 130° C. for 0.5 hr. After cooling to 25° C., the reaction mixture was filtered. After purified by prep-HPLC (column: Waters xbridge 150*25 mm*10 μm; mobile phase. [water (NH₄HCO₃)-ACN]; gradient: 47%-77% B over 18 min), 4-((1-(4-(2-(2-aminopyridin-3-yl)-5-(3-chlorophenyl)-3H-imidazo[4,5-b]pyridin-3-yl)benzyl)piperidin-4-yl)amino)pyrimidine-2-carbonitrile (Example 353, 13.3 mg, yield: 11%) was obtained as a purple solid. MS: m/z=613.4 [M+H]⁺. ¹H NMR (400 MHz, Methanol-d₄) δ 8.22 (d, J=8.4 Hz, 1H), 8.07-8.03 (m, 1H), 8.03-7.93 (m, 4H), 7.58 (d, J=8.0 Hz, 2H), 7.48-7.32 (m, 5H), 6.63-6.56 (m, 1H), 6.48 (dd, J=7.6, 5.2 Hz, 1H), 4.03-3.89 (m, 1H), 3.68 (s, 2H), 3.04-2.93 (m, 2H), 2.35-2.25 (m, 2H), 2.05-1.99 (m, 2H), 1.67-1.58 (m, 2H).

### Example 354: 2-(4-(4-(2-(2-Aminopyridin-3-yl)-5-(1-methyl-6-oxo-1,6-dihydropyridin-3-yl)-3H-imidazo[4,5-b]pyridin-3-yl)benzyl)piperazin-1-yl)pyrimidine-4-carbonitrile

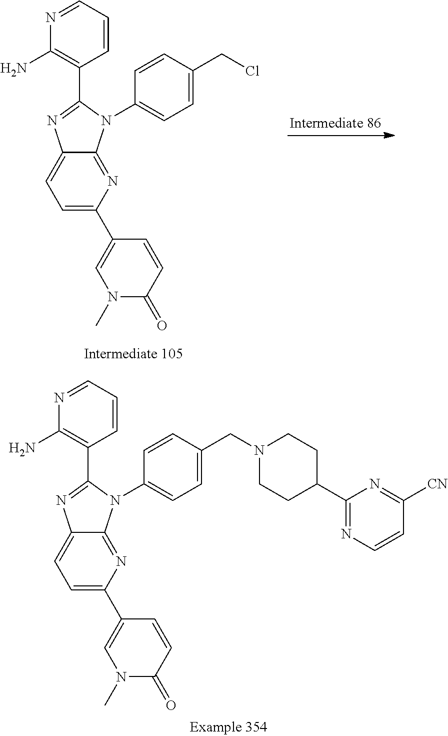 [SMILES: Cn1cc(-c2ccc3nc(-c4cccnc4N)n(-c4ccc(CCl)cc4)c3n2)ccc1=O ; Cn1cc(-c2ccc3nc(-c4cccnc4N)n(-c4ccc(CN5CCC(c6nccc(C#N)n6)CC5)cc4)c3n2)ccc1=O]

To a mixture of Intermediate 105 (50 mg, 113 μmol) and Intermediate 86 (21.4 mg, 113 μmol) in DMF (1 mL) was added DIEA (58.4 mg, 452 μmol). The mixture was stirred at 80° C. for 1 hr. Then it was concentrated and purified by prep-HPLC (column: Phenomenex C18 75*30 mm*3 μm; mobile phase: [water (NH₃H₂O+NH₄HCO₃)-ACN]; gradient: 35%-65% B over 7 min) to give 2-(4-(4-(2-(2-Aminopyridin-3-yl)-5-(1-methyl-6-oxo-1,6-dihydropyridin-3-yl)-3H-imidazo[4,5-b]pyridin-3-yl)benzyl)piperazin-1-yl)pyrimidine-4-carbonitrile (Example 354, 13.8 mg, yield: 21%) as a yellow solid. MS: m/z=596.2 [M+H]⁺. ¹H NMR (400 MHz, Dimethylsulfoxide-d₆) δ 8.65 (d, J=4.8 Hz, 1H), 8.47 (d, J=2.8 Hz, 1H), 8.23 (d, J=8.4 Hz, 1H), 8.09 (dd, J=9.6, 2.8 Hz, 1H), 7.99 (dd, J=5.2, 2.0 Hz, 1H), 7.84 (d, J=8.4 Hz, 1H), 7.55-7.48 (m, 2H), 7.45-7.38 (m, 2H), 7.18-7.10 (m, 2H), 7.02 (br s, 2H), 6.49 (d, J=9.6 Hz, 1H), 6.36 (dd, J=7.6, 5.2 Hz, 1H), 3.82-3.76 (m, 4H), 3.64 (s, 2H), 3.52 (s, 3H), 3.35-3.32 (m, 4H).

### Example 355: 4-((1-(4-(2-(2-Aminopyridin-3-yl)-5-(1-methyl-6-oxo-1,6-dihydropyridin-3-yl)-3H-imidazo[4,5-b]pyridin-3-yl)benzyl)piperidin-4-yl)amino)pyrimidine-2-carbonitrile

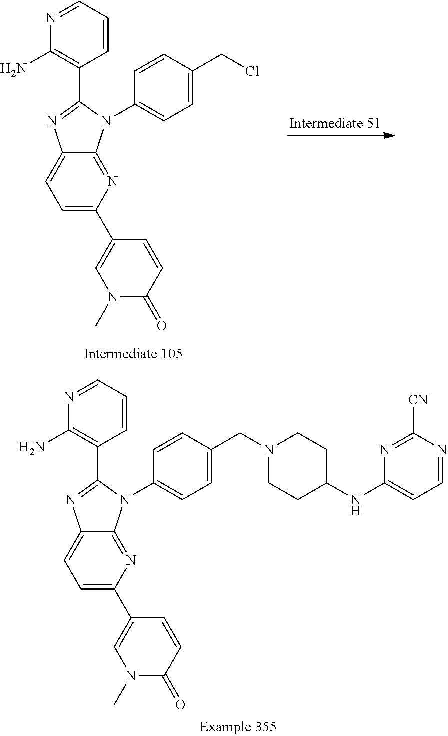 [SMILES: Cn1cc(-c2ccc3nc(-c4cccnc4N)n(-c4ccc(CCl)cc4)c3n2)ccc1=O ; Cn1cc(-c2ccc3nc(-c4cccnc4N)n(-c4ccc(CN5CCC(Nc6ccnc(C#N)n6)CC5)cc4)c3n2)ccc1=O]

To a mixture of Intermediate 105 (150 mg, 339 μmol) and Intermediate 51 (75.7 mg, 373 μmol) in DMF (1 mL) was added DIEA (175 mg, 1.35 mmol). The mixture was stirred at 80° C. for 1 hr. Then it was concentrated and purified by prep-HPLC (column: Phenomenex C18 75*30 mm*3 μm; mobile phase: [water (NH₃H₂O+NH₄HCO₃)-ACN]; gradient: 30%-60% B over 7 min) to give 4-((1-(4-(2-(2-Aminopyridin-3-yl)-5-(1-methyl-6-oxo-1,6-dihydropyridin-3-yl)-3H-imidazo[4,5-b]pyridin-3-yl)benzyl)piperidin-4-yl)amino)pyrimidine-2-carbonitrile (Example 355, 55.4 mg, yield. 27%) as a yellow solid. MS: m/z=610.2 [M+H]⁺. ¹H NMR (400 MHz, Dimethylsulfoxide-d₆) δ 8.47 (d, J=2.4 Hz, 1H), 8.23 (d, J=8.8 Hz, 1H), 8.14-8.00 (m, 3H), 7.99 (dd, J=4.8, 2.0 Hz, 1H), 7.83 (d, J=8.4 Hz, 1H), 7.50-7.45 (m, 2H), 7.44-7.35 (m, 2H), 7.12 (dd, J=8.0, 2.0 Hz, 1H), 7.02 (br s, 2H), 6.67 (d, J=6.0 Hz, 1H), 6.48 (d, J=9.6 Hz, 1H), 6.36 (dd, J=7.6, 4.8 Hz, 1H), 3.91-3.74 (m, 1H), 3.59 (s, 2H), 3.52 (s, 3H), 2.89-2.78 (m, 2H), 2.21-2.07 (m, 2H), 1.95-1.79 (m, 2H), 1.58-1.40 (m, 2H).

### Example 356: 2-((1-(4-(2-(2-Aminopyridin-3-yl)-5-(1-methyl-6-oxo-1,6-dihydropyridin-3-yl)-3H-imidazo[4,5-b]pyridin-3-yl)benzyl)piperidin-4-yl)amino)pyrimidine-4-carbonitrile

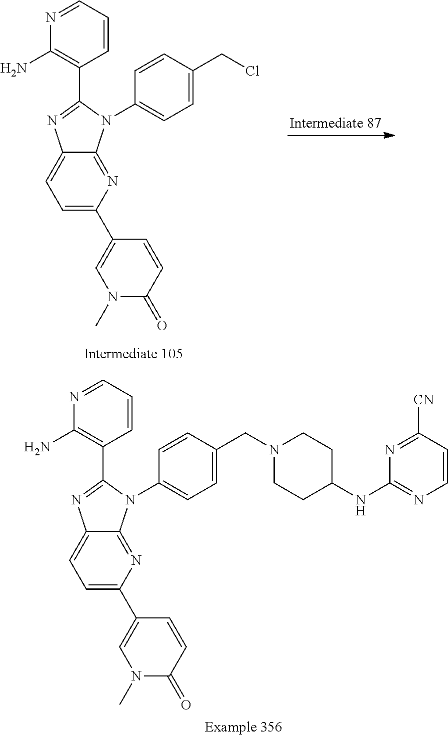 [SMILES: Cn1cc(-c2ccc3nc(-c4cccnc4N)n(-c4ccc(CCl)cc4)c3n2)ccc1=O ; Cn1cc(-c2ccc3nc(-c4cccnc4N)n(-c4ccc(CN5CCC(Nc6nccc(C#N)n6)CC5)cc4)c3n2)ccc1=O]

To a mixture of Intermediate 105 (50 mg, 113 μmol) and Intermediate 87 (25.2 mg, 124 μmol) in DMF (1 mL) was added DIEA (58.4 mg, 452 μmol). The mixture was stirred at 80° C. for 1 hr. Then it was concentrated and purified by prep-HPLC (column: Phenomenex C18 75*30 mm*3 μm; mobile phase: [water (NH₃H₂O+NH₄HCO₃)-ACN]; gradient: 30%-60% B over 7 min) to give 2-((1-(4-(2-(2-Aminopyridin-3-yl)-5-(1-methyl-6-oxo-1,6-dihydropyridin-3-yl)-3H-imidazo[4,5-h]pyridin-3-yl)benzyl)piperidin-4-yl)amino)pyrimidine-4-carbonitrile (Example 356, 7.3 mg, yield: 11%) as a yellow solid. MS: m/z=610.2 [M+H]⁺. ¹H NMR (400 MHz, Dimethylsulfoxide-d₆) δ 8.64-8.51 (m, 1H), 8.46 (d, J=2.0 Hz, 1H), 8.22 (d, J=8.4 Hz, 1H), 8.08 (dd, J=9.6, 2.0 Hz, 1H), 7.99 (d, J=3.6 Hz, 1H), 7.90-7.77 (m, 2H), 7.50-7.44 (m, 2H), 7.43-7.36 (m, 2H), 7.12 (d, J=7.6 Hz, 1H), 7.07 (d, J=4.4 Hz, 1H), 7.03 (br s, 2H), 6.48 (d, J=9.2 Hz, 1H), 6.35 (dd, J=7.6, 5.2 Hz, 1H), 3.80-3.64 (m, 1H), 3.57 (s, 2H), 3.51 (s, 3H), 2.90-2.74 (m, 2H), 2.20-2.00 (m, 2H), 1.91-1.76 (m, 2H), 1.62-1.42 (m, 2H).

### Example 357: 4-((1-(4-(2-(2-Aminopyridin-3-yl)-5-(3-fluorophenyl)-3H-imidazo[4,5-b]pyridin-3-yl)benzyl)piperidin-4-yl)amino)pyrimidine-2-carbonitrile

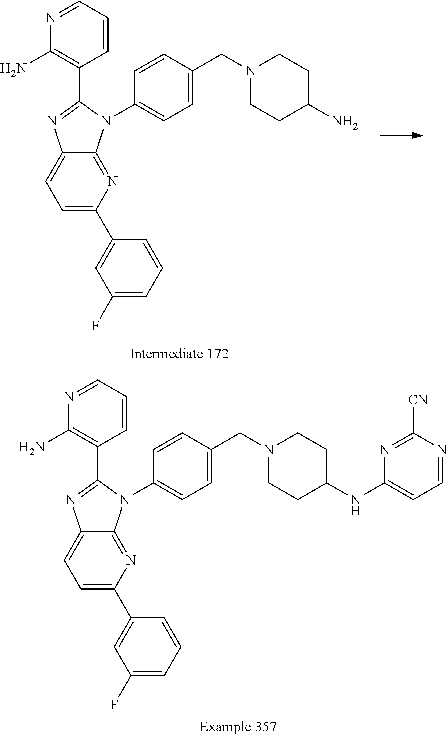 [SMILES: Nc1ncccc1-c1nc2ccc(-c3cccc(F)c3)nc2n1-c1ccc(CN2CCC(N)CC2)cc1 ; N#Cc1nccc(NC2CCN(Cc3ccc(-n4c(-c5cccnc5N)nc5ccc(-c6cccc(F)c6)nc54)cc3)CC2)n1]

A solution of Intermediate 172 (110 mg, 223 μmol), 4-chloropyrimidine-2-carbonitrile (34.2 mg, 245 μmol) and DIEA (86.4 mg, 669 μmol) in NMP (2 mL) were taken up into a microwave tube. The mixture was heated at 130° C. for 0.5 hr using microwave irradiation. After cooling to 25° C., the reaction mixture was filtered. After purified by prep-HPLC (column: Waters xbridge 150×25 mm 10 μm; mobile phase: [water (NH₄HCO₃)-ACN]; gradient: 43%-73% B over 18 min), 4-((1-(4-(2-(2-aminopyridin-3-yl)-5-(3-fluorophenyl)-3H-imidazo[4,5-b]pyridin-3-yl)benzyl)piperidin-4-yl)amino)pyrimidine-2-carbonitrile (Example 357, 11.5 mg, yield: 9%) was obtained as a light-yellow solid. MS: m/z=597.5 [M+H]. ¹H NMR (400 MHz, Methanol-d₄) δ 8.23 (d, J=8.4 Hz, 1H), 8.06-7.96 (m, 3H), 7.89 (d, J=8.4 Hz, 1H), 7.82-7.78 (m, 1H), 7.59 (d, J=8.4 Hz, 2H), 7.50-7.44 (m, 3H), 7.37-7.35 (m, 1H), 7.16-7.09 (m, 1H), 6.66-6.59 (m, 1H), 6.51 (dd, J=7.6, 5.2 Hz, 1H), 3.91-3.89 (m, 1H), 3.70 (s, 2H), 3.04-2.97 (m, 2H), 2.36-2.28 (m, 2H), 2.06-2.01 (m, 2H), 1.68-1.59 (m, 2H); ¹⁹F NMR (400 MHz, Methanol-d₄) δ −115.29.

### Example 358: 2-((1-(4-(2-(2-Aminopyridin-3-yl)-5-(3-fluorophenyl)-3H-imidazo[4,5-b]pyridin-3-yl)benzyl)piperidin-4-yl)amino)pyrimidine-4-carbonitrile

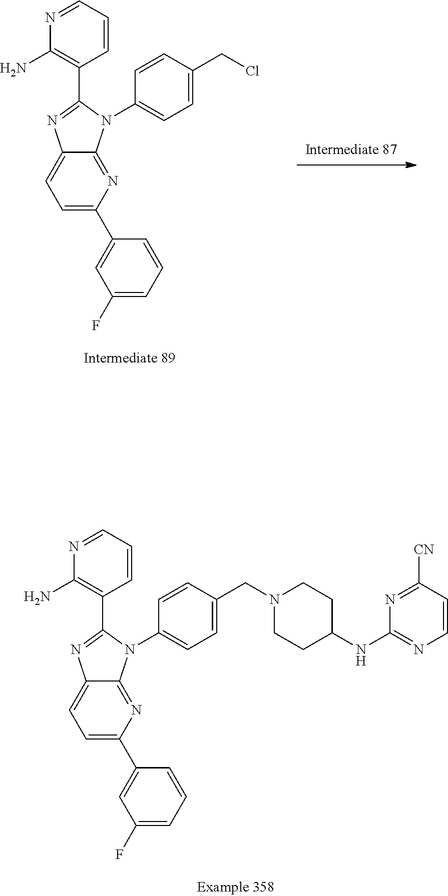 [SMILES: Nc1ncccc1-c1nc2ccc(-c3cccc(F)c3)nc2n1-c1ccc(CCl)cc1 ; N#Cc1ccnc(NC2CCN(Cc3ccc(-n4c(-c5cccnc5N)nc5ccc(-c6cccc(F)c6)nc54)cc3)CC2)n1]

To a solution of Intermediate 89 (200 mg HCl salt, 429 μmol) and Intermediate 87 (136 mg TFA salt, 429 μmol) in DMF (5 mL) was added NaI (32.1 mg, 214 μmol) and K₂CO₃ (296 mg, 2.14 mmol). The mixture was stirred at 50° C. for 1 hr. The reaction mixture was filtered. After purified by prep-HPLC (column: Waters Xbridge C18 150×50 mm×10 μm; mobile phase: [water (NH₄HCO₃)-ACN]; gradient: 52%-82% B over 10 min), 2-((1-(4-(2-(2-aminopyridin-3-yl)-5-(3-fluorophenyl)-3H-imidazo[4,5-b]pyridin-3-yl)benzyl)piperidin-4-yl)amino)pyrimidine-4-carbonitrile (Example 358, 21.2 mg, yield: 8%) was obtained as a yellow solid. MS: m/z=597.3 [M+H]⁺. ¹H NMR (400 MHz, Dimethylsulfoxide-d₆) δ 8.62 (d, J=4.8 Hz, 1H), 8.28 (d, J=8.4 Hz, 1H), 8.05 (d, J=8.4 Hz, 1H), 8.01-8.01 (m, 1H), 7.90 (d, J=7.6 Hz, 1H), 7.82 (d, J=10.8 Hz, 1H), 7.57-7.48 (m, 4H), 7.44 (d, J=8.4 Hz, 2H), 7.23-7.19 (m, 2H), 7.10 (d, J=4.4 Hz, 1H), 7.02 (br s, 2H), 6.41 (dd, J=7.6, 4.8 Hz, 1H), 4.48-4.41 (m, 2H), 3.87 (s, 2H), 3.17-3.11 (m, 2H), 2.77-2.74 (m, 1H), 1.94-1.89 (m, 2H), 1.31-1.24 (m, 2H). ¹⁹F NMR (400 MHz, Dimethylsulfoxide-d₆) δ −112.913.

### Example 359: 4-((1-(4-(2-(2-Aminopyridin-3-yl)-5-(1-methyl-2-oxo-1,2-dihydropyridin-4-yl)-3H-imidazo[4,5-b]pyridin-3-yl)benzyl)piperidin-4-yl)amino)pyrimidine-2-carbonitrile

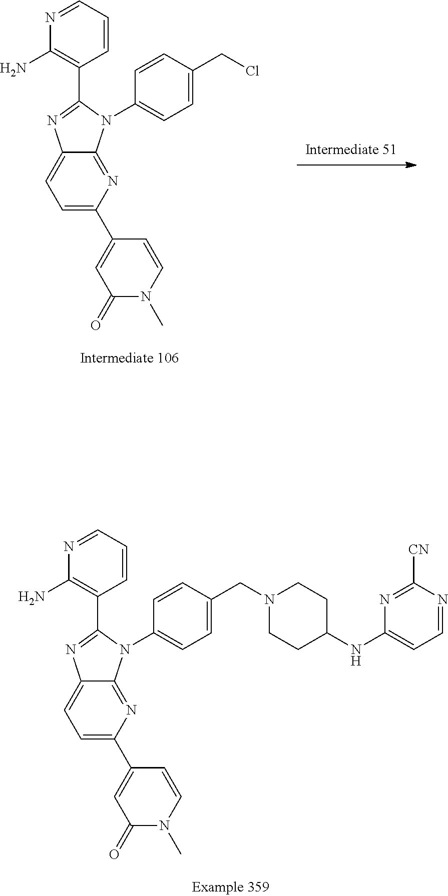 [SMILES: Cn1ccc(-c2ccc3nc(-c4cccnc4N)n(-c4ccc(CCl)cc4)c3n2)cc1=O ; Cn1ccc(-c2ccc3nc(-c4cccnc4N)n(-c4ccc(CN5CCC(Nc6ccnc(C#N)n6)CC5)cc4)c3n2)cc1=O]

To a mixture of Intermediate 106 (100 mg, 226 μmol) and Intermediate 51 (50.5 mg, 248 μmol) in DMF (1 mL) was added DIEA (117 mg, 903 μmol). The mixture was stirred at 80° C. for 1 hr. The mixture was concentrated and purified by prep-HPLC (column: Phenomenex C18 75*30 mm*3 μm; mobile phase: [water (NH₃H₂O+NH₄HCO₃)-ACN]; gradient: 30%-60% B over 7 min) to give 4-((1-(4-(2-(2-aminopyridin-3-yl)-5-(1-methyl-2-oxo-1,2-dihydropyridin-4-yl)-3H-imidazo[4,5-b]pyridin-3-yl)benzyl)piperidin-4-yl)amino)pyrimidine-2-carbonitrile (Example 359, 85.7 mg, yield: 62%) as a yellow solid. MS: m/z=610.2 [M+H]⁺. ¹H NMR (400 MHz, Dimethylsulfoxide-d₆) δ 8.28 (d, J=8.4 Hz, 1H), 8.12-7.93 (m, 4H), 7.75 (d, J=7.2 Hz, 1H), 7.53-7.39 (m, 4H), 7.17 (dd, J=7.6, 1.6 Hz, 1H), 7.08-6.98 (m, 3H), 6.85 (dd, J=7.2, 2.0 Hz, 1H), 6.67 (d, J=6.0 Hz, 1H), 6.38 (dd, J=7.6, 4.8 Hz, 1H), 3.87-3.73 (m, 1H), 3.59 (s, 2H), 3.43 (s, 3H), 2.89-2.79 (m, 2H), 2.22-2.08 (m, 2H), 1.95-1.81 (m, 2H), 1.59-1.39 (m, 2H).

### Example 360: 2-((1-(4-(2-(2-Aminopyridin-3-yl)-5-(1-methyl-2-oxo-1,2-dihydropyridin-4-yl)-3H-imidazo[4,5-b]pyridin-3-yl)benzyl)piperidin-4-yl)amino)pyrimidine-4-carbonitrile

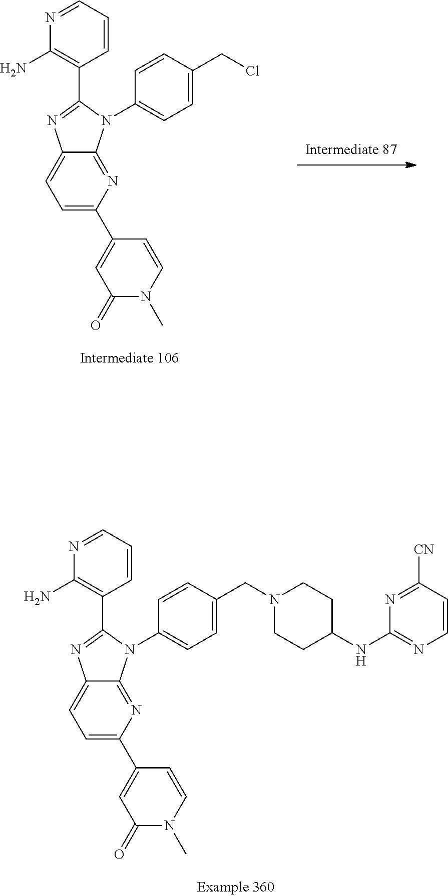 [SMILES: Cn1ccc(-c2ccc3nc(-c4cccnc4N)n(-c4ccc(CCl)cc4)c3n2)cc1=O ; Cn1ccc(-c2ccc3nc(-c4cccnc4N)n(-c4ccc(CN5CCC(Nc6nccc(C#N)n6)CC5)cc4)c3n2)cc1=O]

To a mixture of Intermediate 106 (50 mg, 113 μmol) and Intermediate 87 (25.2 mg, 124 μmol) in DMF (1 mL) was added DIEA (58.3 mg, 452 μmol), the mixture was stirred at 80° C. for 1 hr. Then it was concentrated. After purified byprep-HPLC (column: Phenomenex C18 75*30 mm*3 μm; mobile phase: [water (NH₃H₂O+NH₄HCO₃)-ACN]; gradient: 30%-60% B over 7 min), 2-((1-(4-(2-(2-aminopyridin-3-yl)-5-(1-methyl-2-oxo-1,2-dihydropyridin-4-yl)-3H-imidazo[4,5-b]pyridin-3-yl)benzyl)piperidin-4-yl)amino)pyrimidine-4-carbonitrile (Example 360, 17.2 mg, yield: 25%) as a yellow solid. MS: m/z=610.1 [M+H]⁺. ¹H NMR (400 MHz, Dimethylsulfoxide-d₆) δ 8.61-8.49 (m, 1H), 8.29 (d, J=8.4 Hz, 1H), 8.09-7.97 (m, 2H), 7.89-7.82 (m, 1H), 7.75 (d, J=7.2 Hz, 1H), 7.52-7.40 (m, 4H), 7.18 (dd, J=7.6, 1.2 Hz, 1H), 7.09-6.99 (m, 4H), 6.86 (dd, J=7.2, 2.0 Hz, 1H), 6.38 (dd, J=7.6, 4.8 Hz, 1H), 3.78-3.64 (m, 1H), 3.58 (s, 2H), 3.43 (s, 3H), 2.88-2.80 (m, 2H), 2.16-2.03 (m, 2H), 1.89-1.80 (m, 2H), 1.62-1.48 (m, 2H).

### Example 361: 4-(7-(4-(2-(2-Aminopyridin-3-yl)-5-phenyl-3H-imidazo[4,5-b]pyridin-3-yl)benzyl)-2,7-diazaspiro[3.5]nonan-2-yl)pyrimidine-2-carbonitrile

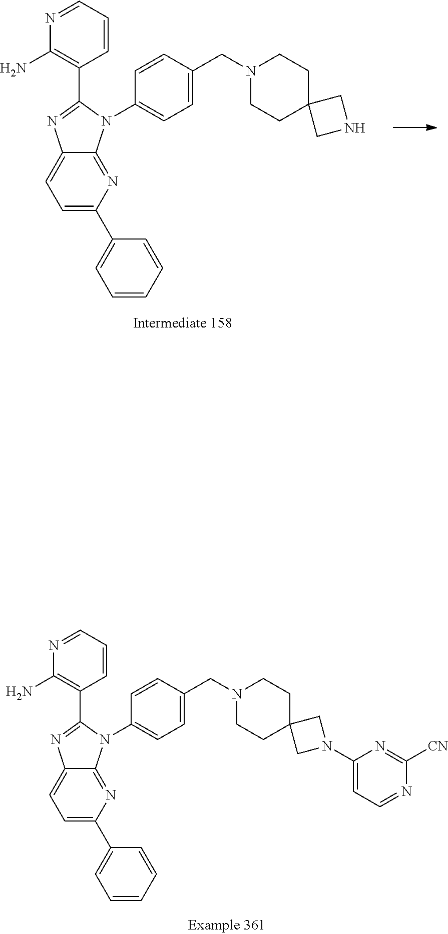 [SMILES: Nc1ncccc1-c1nc2ccc(-c3ccccc3)nc2n1-c1ccc(CN2CCC3(CC2)CNC3)cc1 ; N#Cc1nccc(N2CC3(CCN(Cc4ccc(-n5c(-c6cccnc6N)nc6ccc(-c7ccccc7)nc65)cc4)CC3)C2)n1]

To a solution of Intermediate 158 (100 mg, 199 μmol) and 4-chloropyrimidine-2-carbonitrile (30.6 mg, 219 μmol) in DMF (2 mL) were added NaI (2.99 mg, 19.9 μmol) and K₂CO₃ (55.1 mg, 399 μmol). The mixture was stirred at 80° C. for 2 hr. The reaction mixture was filtered. After purified by prep-HPLC (column: Phenomenex C18 150*25 mm*10 μm; mobile phase: [water (NH₄HCO₃)-ACN]; gradient: 50%-80% B over 10 min), 4-(7-(4-(2-(2-aminopyridin-3-yl)-5-phenyl-3H-imidazo[4,5-b]pyridin-3-yl)benzyl)-2,7-diazaspiro[3.5]nonan-2-yl)pyrimidine-2-carbonitrile (Example 361, 19.9 mg, yield: 16%) was obtained as a light-yellow solid. MS: m/z=605.4 [M+H]⁺. ¹H NMR (400 MHz, Dimethylsulfoxide-d₆) δ 8.29-8.20 (m, 2H), 8.04-7.97 (m, 4H), 7.50-7.44 (m, 6H), 7.42-7.37 (m, 1H), 7.17 (d, J=7.6 Hz, 1H), 7.03 (br s, 2H), 6.63 (d, J=6 Hz, 1H), 6.38 (dd, J=7.6, 4.8 Hz, 1H), 3.80 (s, 4H), 3.56 (s, 2H), 3.31-3.27 (m, 2H), 2.37-2.35 (m, 2H), 1.82-1.76 (m, 4H).

### Example 362: 4-((7-(4-(2-(2-Aminopyridin-3-yl)-5-phenyl-3H-imidazo[4,5-b]pyridin-3-yl)benzyl)-7-azaspiro[3.5]nonan-2-yl)amino)pyrimidine-2-carbonitrile

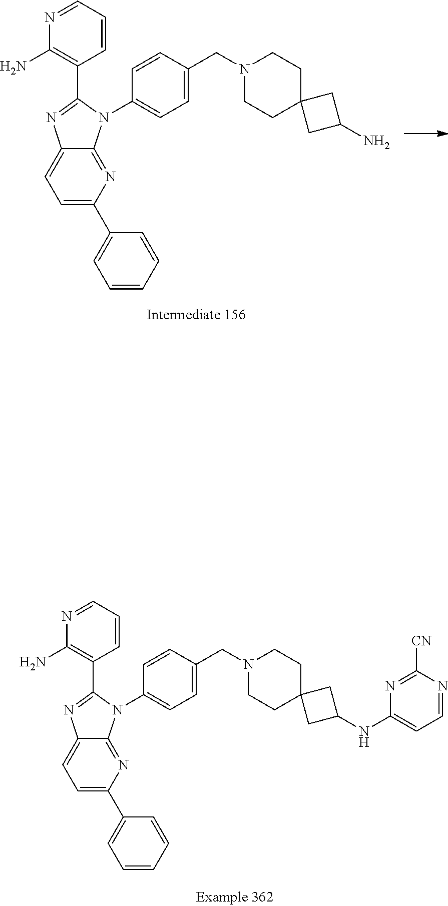 [SMILES: Nc1ncccc1-c1nc2ccc(-c3ccccc3)nc2n1-c1ccc(CN2CCC3(CC2)CC(N)C3)cc1 ; N#Cc1nccc(NC2CC3(CCN(Cc4ccc(-n5c(-c6cccnc6N)nc6ccc(-c7ccccc7)nc65)cc4)CC3)C2)n1]

To a solution of Intermediate 156 (100 mg, 194 μmol) and 4-chloropyrimidine-2-carbonitrile (29.8 mg, 213 μmol) in DMF (2 mL) were added NaI (2.91 mg, 19.4 μmol) and K₂CO₃ (53.6 mg, 388 μmol). The mixture was stirred at 80° C. for 2 hr. The reaction mixture was filtered and purified by prep-HPLC (column. Phenomenex C18 150*25 mm*10 um; mobile phase: [water (NH₄HCO₃)-ACN]; gradient: 42%-72% B over 14 min) to give 4-((7-(4-(2-(2-aminopyridin-3-yl)-5-phenyl-3H-imidazo[4,5-b]pyridin-3-yl)benzyl)-7-azaspiro[3.5]nonan-2-yl)amino)pyrimidine-2-carbonitrile (Example 362, 3.0 mg, yield: 1.2%) as a light-yellow solid. MS: m/z=619.4 [M+H]⁺. ¹H NMR (400 MHz, Dimethylsulfoxide-d₆) δ 8.50-8.32 (m, 1H), 8.28 (d, J=8.4 Hz, 1H), 8.05-7.96 (m, 4H), 7.50-7.43 (m, 7H), 7.42-7.36 (m, 1H), 7.16 (dd, J=8.0, 2.0 Hz, 1H), 7.04 (br s, 2H), 6.65 (d, J=6.0 Hz, 1H), 6.37 (dd, J=7.6, 4.8 Hz, 1H), 4.48-4.17 (m, 1H), 3.54 (s, 2H), 3.31-3.29 (m, 2H), 2.35-2.31 (m, 2H), 2.30-2.23 (m, 4H), 1.67-1.64 (m, 2H), 1.58-1.54 (m, 2H).

### Example 363: 4-(8-(4-(2-(2-Aminopyridin-3-yl)-5-phenyl-3H-imidazo[4,5-b]pyridin-3-yl)benzyl)-2,8-diazaspiro[4.5]decan-2-yl)-1,3,5-triazine-2-carbonitrile

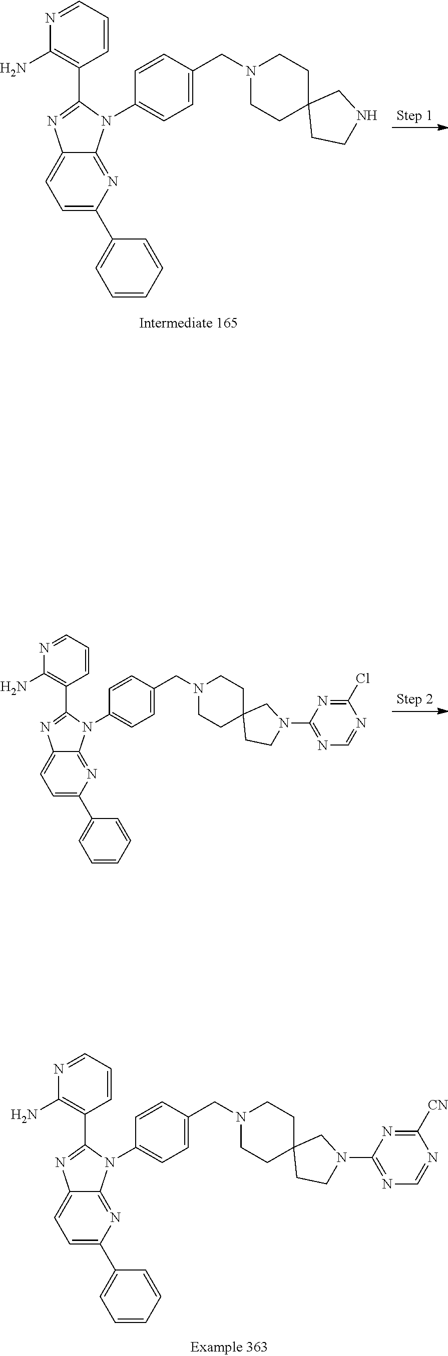 [SMILES: Nc1ncccc1-c1nc2ccc(-c3ccccc3)nc2n1-c1ccc(CN2CCC3(CCNC3)CC2)cc1 ; Nc1ncccc1-c1nc2ccc(-c3ccccc3)nc2n1-c1ccc(CN2CCC3(CC2)CCN(c2ncnc(Cl)n2)C3)cc1 ; N#Cc1ncnc(N2CCC3(CCN(Cc4ccc(-n5c(-c6cccnc6N)nc6ccc(-c7ccccc7)nc65)cc4)CC3)C2)n1]

### Step 1: 3-(3-(4-((2-(4-Chloro-1,3,5-triazin-2-yl)-2,8-diazaspiro[4.5]decan-8-yl)methyl)phenyl)-5-phenyl-3H-imidazo[4,5-b]pyridin-2-yl)pyridin-2-amine

To a solution of Intermediate 168 (80 mg, 155 μmol) in THF (8 mL) and H₂O (2 mL) was added DIEA (60 mg, 465 μmol). Then 4-Chloro-1,3,5-triazine-2-carbonitrile (28 mg, 186 μmol) in THF (2 mL) was added into the mixture at 0° C. The mixture was stirred at 0° C. for 1 hr. The reaction mixture was extracted with CH₂Cl₂ (30 mL). The organic phase was separated, dried over anhydrous Na₂SO₄, filtered and concentrated under reduced pressure. After purified by prep-TLC (CH₂Cl₂:MeOH=10:1), 3-(3-(4-((2-(4-chloro-1,3,5-triazin-2-yl)-2,8-diazaspiro[4.5]decan-8-yl)methyl)phenyl)-5-phenyl-3H-imidazo[4,5-b]pyridin-2-yl)pyridin-2-amine (4.2 mg, yield: 4%) was obtained as a yellow solid. MS: m/z=629.3 [M+H]. ¹H NMR (400 MHz, Methanol-d₄) δ 8.32 (d, J=6.8 Hz, 1H), 8.19 (d, J=8.4 Hz, 1H), 8.07-7.99 (m, 2H), 7.99-7.91 (m, 2H), 7.58-7.51 (m, 2H), 7.48-7.30 (m, 6H), 6.52-6.42 (m, 1H), 3.68-3.60 (m, 4H), 3.48 (d, J=10.0 Hz, 2H), 2.69-2.59 (m, 2H), 2.52-2.41 (m, 2H), 1.95-1.86 (m, 2H), 1.69-1.64 (m, 4H).

### Step 2: 4-(8-(4-(2-(2-Aminopyridin-3-yl)-5-phenyl-3H-imidazo[4,5-b]pyridin-3-yl)benzyl)-2,8-diazaspiro[4.5]decan-2-yl)-1,3,5-triazine-2-carbonitrile

To a solution 3-(3-(4-((2-(4-chloro-1,3,5-triazin-2-yl)-2,8-diazaspiro[4.5]decan-8-yl)methyl)phenyl)-5-phenyl-3H-imidazo[4,5-b]pyridin-2-yl)pyridin-2-amine (80 mg, 127 μmol) in DMSO (1 mL) was added KCN (0.1 g, 1.5 mmol) and DABCO (2.9 mg, 25 μmol). The mixture was stirred at 25° C. for 16 hr. The reaction mixture was poured into water (5 mL) and extracted with CH₂Cl₂ (6 mL). The combined organic layers were washed with brine (2 ml) and dried over anhydrous Na₂SO₄, filtered and concentrated to dryness. After purified by prep-TLC (CH₂Cl₂:MeOH=10:1), 4-(8-(4-(2-(2-aminopyridin-3-yl)-5-phenyl-3H-imidazo[4,5-b]pyridin-3-yl)benzyl)-2,8-diazaspiro[4.5]decan-2-yl)-1,3,5-triazine-2-carbonitrile (Example 363, 4.4 mg, yield: 5%) was obtained as a yellow solid. MS: m/z=620.3 [M+H]⁺. ¹H NMR (400 MHz, Chloroform-d) δ 8.55 (d, J=11.2 Hz, 1H), 8.14 (d, J=8.4 Hz, 1H), 8.06 (d, J=5.2 Hz, 1H), 8.01 (d, J=7.6 Hz, 2H), 7.81 (d, J=8.0 Hz, 1H), 7.65-7.60 (m, 1H), 7.51-7.39 (m, 6H), 7.10 (d, J=8.0 Hz, 1H), 6.69 (br s, 2H), 6.37 (dd, J=7.6, 4.8 Hz, 1H), 3.93-3.76 (m, 2H), 3.71-3.66 (m, 2H), 3.52 (s, 2H), 2.88-2.49 (m, 4H), 2.05-1.92 (m, 6H).

### Example 364: 4-(9-(4-(2-(2-Aminopyridin-3-yl)-5-phenyl-3H-imidazo[4,5-b]pyridin-3-yl)benzyl)-3,9-diazaspiro[5.5]undecan-3-yl)pyrimidine-2-carbonitrile

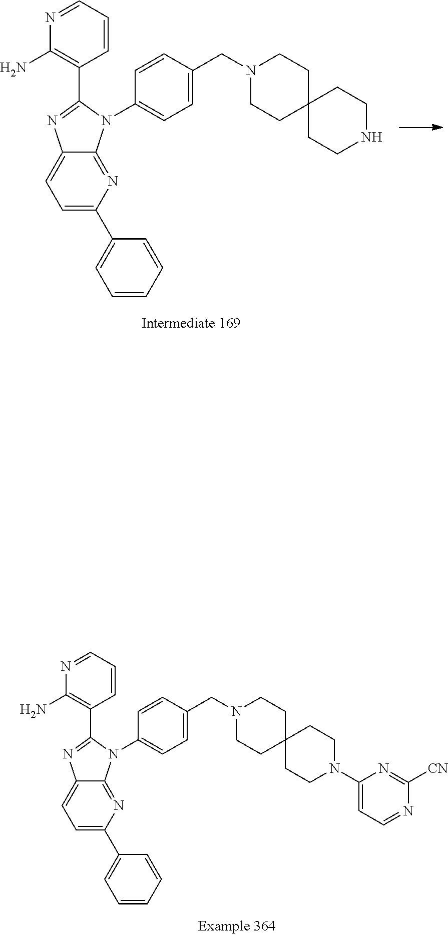 [SMILES: Nc1ncccc1-c1nc2ccc(-c3ccccc3)nc2n1-c1ccc(CN2CCC3(CCNCC3)CC2)cc1 ; N#Cc1nccc(N2CCC3(CCN(Cc4ccc(-n5c(-c6cccnc6N)nc6ccc(-c7ccccc7)nc65)cc4)CC3)CC2)n1]

A mixture of Intermediate 169 (80 mg, 151 μmol), 4-chloropyrimidine-2-carbonitrile (23 mg, 166 μmol) and DIEA (58 mg, 453 μmol) in NMP (2 mL) were taken up into a microwave tube. The sealed tube was heated at 130° C. for 0.5 hr under microwave. The reaction mixture was poured into H₂O (10 mL) at 0° C. and extracted with CH₂Cl₂ (45 mL). The organic layer was dried over anhydrous Na₂SO₄, filtered and concentrated under reduced pressure. After purified by prep-HPLC (column: Phenomenex C18 150*25 mm*10 μm; mobile phase: [water (NH₄HCO₃)-ACN]; gradient: 48%-78% B over 14 min), 4-(9-(4-(2-(2-aminopyridin-3-yl)-5-phenyl-3H-imidazo[4,5-b]pyridin-3-yl)benzyl)-3,9-diazaspiro[5.5]undecan-3-yl)pyrimidine-2-carbonitrile (Example 364, 7.5 mg, yield: 7.7%) was obtained as a pink lyophilized powder. MS: m/z=633.5 [M+H]⁺. ¹H NMR (400 MHz, Dimethylsulfoxide-d₆) δ. 8.27 (d, J=8.4 Hz, 1H), 8.21 (d, J=6.8 Hz, 1H), 8.05-7.96 (m, 4H), 7.48-7.38 (m, 7H), 7.15 (dd, J=7.6, 1.6 Hz, 1H), 7.08 (d, J=6.8 Hz, 1H), 7.03 (br s, 2H), 6.36 (dd, J=7.6, 4.8 Hz, 1H), 3.68-3.61 (m, 2H), 3.59 (s, 2H), 3.31-3.27 (m, 2H), 2.44-2.38 (m, 4H), 1.56-1.45 (m, 8H).

### Example 365: 4-(9-(4-(2-(2-Aminopyridin-3-yl)-5-phenyl-3H-imidazo[4,5-b]pyridin-3-yl)benzyl)-3,9-diazaspiro[5.5]undecan-3-yl)-1,3,5-triazine-2-carbonitrile

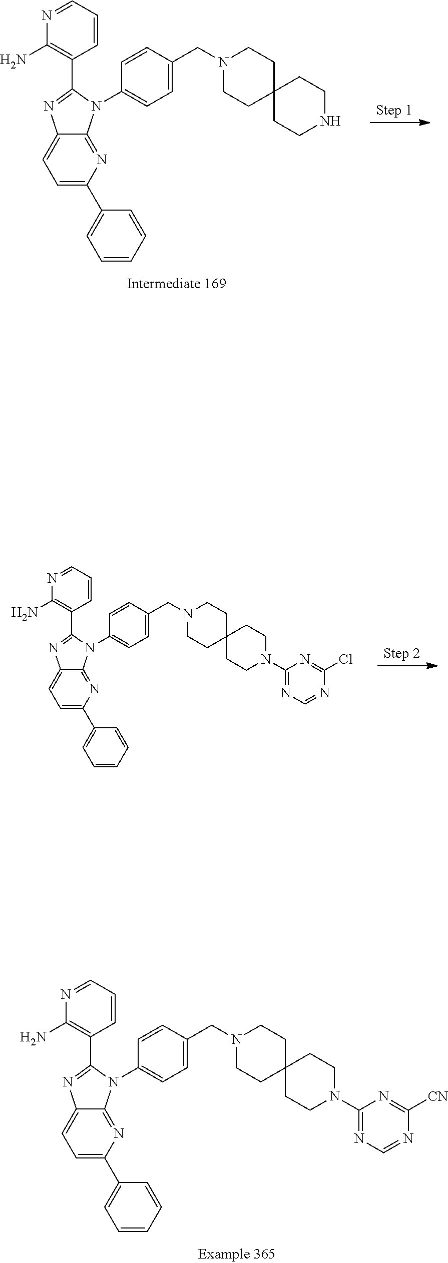 [SMILES: Nc1ncccc1-c1nc2ccc(-c3ccccc3)nc2n1-c1ccc(CN2CCC3(CCNCC3)CC2)cc1 ; Nc1ncccc1-c1nc2ccc(-c3ccccc3)nc2n1-c1ccc(CN2CCC3(CC2)CCN(c2ncnc(Cl)n2)CC3)cc1 ; N#Cc1ncnc(N2CCC3(CCN(Cc4ccc(-n5c(-c6cccnc6N)nc6ccc(-c7ccccc7)nc65)cc4)CC3)CC2)n1]

### Step 1: 3-(3-(4-((9-(4-Chloro-1,3,5-triazin-2-yl)-3,9-diazaspiro[5.5]undecan-3-yl)methyl)phenyl)-5-phenyl-3H-imidazo[4,5-b]pyridin-2-yl)pyridin-2-amine

To a solution of Intermediate 169 (80 mg, 151 μmol) in THF (8 mL) and H₂O (2 mL) was added DIEA (59 mg, 453 μmol). The mixture was stirred at 0° C. for 10 min. Then 4-chloro-1,3,5-triazine-2-carbonitrile (27 mg, 181 μmol) in THF (1.5 mL) was added to the mixture. The mixture was stirred at 0° C. for 1 hr. The reaction mixture was extracted with CH₂Cl₂ (30 mL). The organic phase layers were dried over anhydrous Na₂SO₄, filtered and concentrated under reduced pressure. After purified by prep-TLC (CH₂Cl₂:MeOH=10:1), 3-(3-(4-((9-(4-chloro-1,3,5-triazin-2-yl)-3,9-diazaspiro[5.5]undecan-3-yl)methyl)phenyl)-5-phenyl-3H-imidazo[4,5-b]pyridin-2-yl)pyridin-2-amine (13.8 mg, yield: 14%) was obtained as a yellow solid. MS: m/z=643.2 [M+H]⁺. ¹H NMR (400 MHz, Methanol-d₆) δ 8.29 (s, 1H), 0.8.19 (d, J=8.4 Hz, 1H), 8.05-8.01 (m, 2H), 8.00-7.96 (m, 1H), 7.96-7.88 (m, 1H), 8.19 (d, J=8.4 Hz, 2H), 7.47-7.31 (m, 6H), 6.51-6.43 (m, 1H), 3.90-3.86 (m, 2H), 3.85-3.80 (m, 2H), 3.65 (s, 2H), 2.61-2.51 (m, 4H), 1.68-1.63 (m, 4H), 1.59-1.54 (m, 4H).

### Step 2: 4-(9-(4-(2-(2-Aminopyridin-3-yl)-5-phenyl-3H-imidazo[4,5-b]pyridin-3-yl)benzyl)-3,9-diazaspiro[5.5]undecan-3-yl)-1,3,5-triazine-2-carbonitrile

To a solution of 3-(3-(4-((9-(4-chloro-1,3,5-triazin-2-yl)-3,9-diazaspiro[5.5]undecan-3-yl)methyl)phenyl)-5-phenyl-3H-imidazo[4,5-b]pyridin-2-yl)pyridin-2-amine (80 mg, 124 μmol) in DMSO (1 mL) was added KCN (57 mg, 871 μmol) and DABCO (2.8 mg, 25 μmol). The mixture was stirred at 25° C. for 16 hr. The reaction mixture was poured into water (5 mL). The resulting mixture was extracted with CH₂Cl₂ (6 mL). The combined organic layers were washed with brine (2 ml) and dried over anhydrous Na₂SO₄, filtered and concentrated to dryness. After purified by prep-TLC (CH₂Cl₂:MeOH=10:1), 4-(9-(4-(2-(2-aminopyridin-3-yl)-5-phenyl-3H-imidazo[4,5-b]pyridin-3-yl)benzyl)-3,9-diazaspiro[5.5]undecan-3-yl)-1,3,5-triazine-2-carbonitrile (Example 365, 10.8 mg, yield: 13%) was obtained as a yellow solid. MS: m/z=634.3 [M+H]⁺. 1H NMR (400 MHz, Chloroform-d) δ 8.53 (s, 1H), 8.14 (d, J=8.4 Hz, 1H), 8.09-8.05 (m, 1H), 8.02 (d, J=7.6 Hz, 2H), 7.81 (d, J=8.4 Hz, 1H), 7.61-7.51 (m, 2H), 7.47-7.41 (m, 4H), 7.40-7.36 (m, 1H), 7.09 (d, J=6.4 Hz, 1H), 6.60 (br s, 2H), 6.36 (dd, J=7.6, 4.8 Hz, 1H), 3.87-3.81 (m, 4H), 3.79-3.64 (m, 2H), 2.77-2.51 (m, 4H), 1.61-1.60 (m, 8H).

### Example 366: 4-(6-(4-(2-(2-Aminopyridin-3-yl)-5-phenyl-3H-imidazo[4,5-b]pyridin-3-yl)benzyl)-2,6-diazaspiro[3.3]heptan-2-yl)pyrimidine-2-carbonitrile

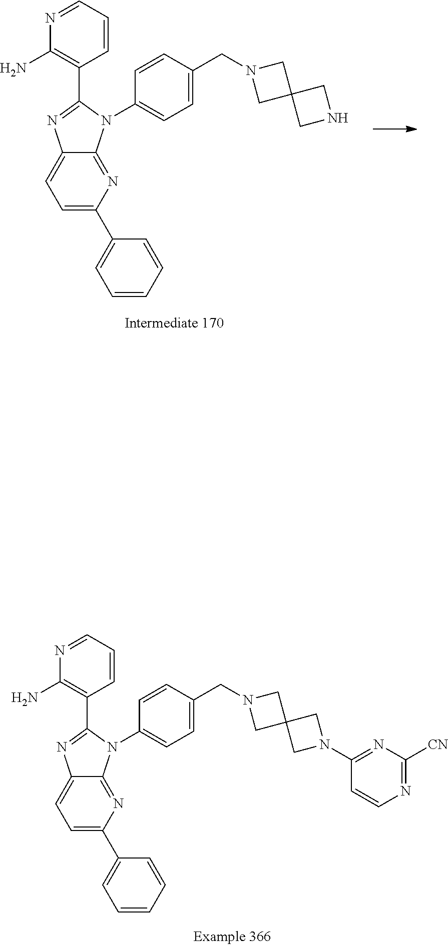 [SMILES: Nc1ncccc1-c1nc2ccc(-c3ccccc3)nc2n1-c1ccc(CN2CC3(CNC3)C2)cc1 ; N#Cc1nccc(N2CC3(CN(Cc4ccc(-n5c(-c6cccnc6N)nc6ccc(-c7ccccc7)nc65)cc4)C3)C2)n1]

To a solution of Intermediate 170 (80 mg, 169 μmol) in DMF (2 mL) were added 4-chloropyrimidine-2-carbonitrile (26 mg, 186 μmol) and DIEA (66 mg, 507 μmol). The mixture was stirred at 80° C. for 1.5 hr. The reaction mixture was poured into H₂O (15 mL), extracted with EtOAc (45 mL). The organic layers were dried over anhydrous Na₂SO₄, filtered and concentrated under reduced pressure. After purified by prep-HPLC (column: Phenomenex C18 150*25 mm*10 μm; mobile phase: [water (NH₄HCO₃)-ACN]; gradient: 37%-67% B over 14 min), 4-(6-(4-(2-(2-aminopyridin-3-yl)-5-phenyl-3H-imidazo[4,5-b]pyridin-3-yl)benzyl)-2,6-diazaspiro[3.3]heptan-2-yl)pyrimidine-2-carbonitrile (Example 366, 6.1 mg, yield: 6%) was obtained as an off-white lyophilized powder. MS: m/z=577.3 [M+H]⁺. ¹H NMR (400 MHz, Dimethylsulfoxide-d₆) δ 8.32-8.14 (m, 2H), 8.08-7.94 (m, 4H), 7.50-7.37 (m, 7H), 7.15 (d, J=7.61 Hz, 1H), 7.01 (br s, 2H), 6.62 (d, J=5.6 Hz, 1H), 6.46-6.32 (m, 1H), 4.20 (s, 4H), 3.64 (s, 2H), 3.38 (s, 4H).

### Example 367: 4-((3aS,6aS)-5-(4-(2-(2-aminopyridin-3-yl)-5-phenyl-3H-imidazo[4,5-b]pyridin-3-yl)benzyl)hexahydropyrrolo[3,4-c]pyrrol-2(1H)-yl)pyrimidine-2-carbonitrile

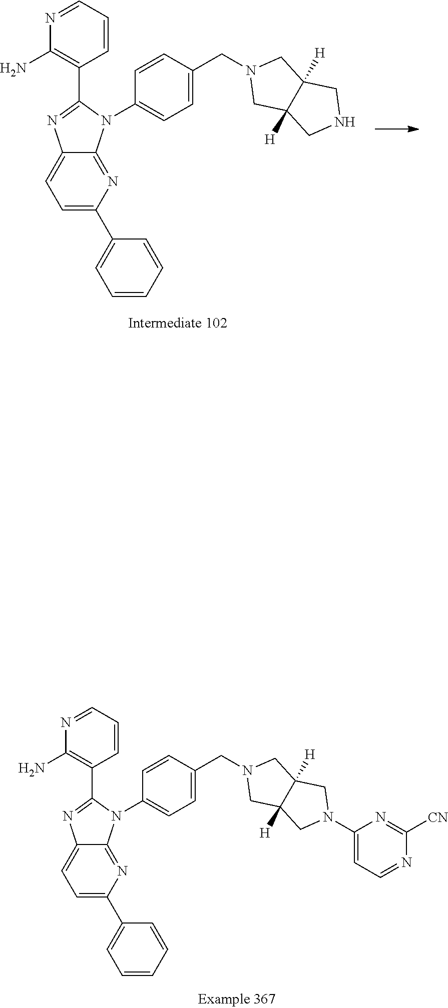 [SMILES: Nc1ncccc1-c1nc2ccc(-c3ccccc3)nc2n1-c1ccc(CN2C[C@H]3CNC[C@@H]3C2)cc1 ; N#Cc1nccc(N2C[C@@H]3CN(Cc4ccc(-n5c(-c6cccnc6N)nc6ccc(-c7ccccc7)nc65)cc4)C[C@H]3C2)n1]

To a solution of Intermediate 102 (81.0 mg HCl salt, 155 μmol) in NMP (0.8 mL) were added 4-chloropyrimidine-2-carbonitrile (21.6 mg, 155 μmol) and DIEA (59.9 mg, 81 μL). The mixture was stirred at 5° C. for 10 min. The residue was purified by pre-HPLC column: Welch Xtimate C18 150×25 mm×5 μm; mobile phase: [water (NH₃H₂O+NH₄HCO₃)-ACN]; gradient: 60%-90% B over 7 min to give 4-((3aS,6aS)-5-(4-(2-(2-aminopyridin-3-yl)-5-phenyl-3H-imidazo[4,5-b]pyridin-3-yl)benzyl)hexahydropyrrolo[3,4-c]pyrrol-2(1H)-yl)pyrimidine-2-carbonitrile (Example 367, 11.0 mg, yield: 12%) as a white solid. MS: m/z=591.3 [M+H]⁺. ¹H NMR (400 MHz, Dimethylsulfoxide-d₆) δ 8.27 (d, J=8.0 Hz, 1H), 8.22 (d, J=6.4 Hz, 1H), 8.06-8.01 (m, 2H), 8.01-7.97 (m, 2H), 7.53-7.37 (m, 7H), 7.17 (dd, J=7.6, 1.6 Hz, 1H), 7.03 (br s, 2H), 6.74 (d, J=6.4 Hz, 1H), 6.39 (dd, J=7.6, 4.8 Hz, 1H), 4.02-3.91 (m, 2H), 3.83 (dd, J=10.8, 6.8 Hz, 1H), 3.61 (dd, 1=9.2, 6.4 Hz, 1H), 3.13-3.07 (m, 2H), 2.95-2.88 (m, 2H), 2.72-2.62 (m, 2H), 2.44-2.29 (m, 2H).

### Example 368: 4-(4-(4-(2-(2-Aminopyridin-3-yl)-5-(4-fluorophenyl)-3H-imidazo[4,5-b]pyridin-3-yl)benzyl)piperazin-1-yl)-1,3,5-triazine-2-carbonitrile

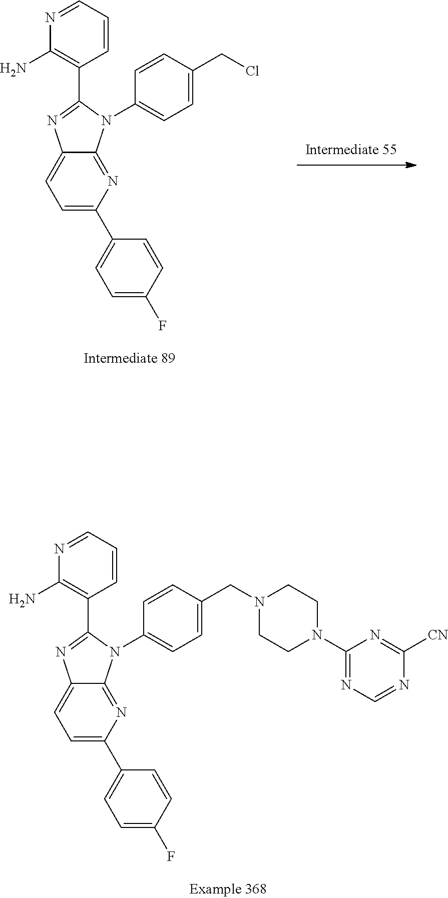 [SMILES: Nc1ncccc1-c1nc2ccc(-c3ccc(F)cc3)nc2n1-c1ccc(CCl)cc1 ; N#Cc1ncnc(N2CCN(Cc3ccc(-n4c(-c5cccnc5N)nc5ccc(-c6ccc(F)cc6)nc54)cc3)CC2)n1]

To a solution of Intermediate 89 (300 mg, 643 μmol) and Intermediate 55 (196 mg, 643 μmol) in DMF (2 mL) were added NaI (48.2 mg, 322 μmol) and K₂CO₃ (445 mg, 3.22 mmol). The mixture was stirred at 50° C. for 0.5 hr. The reaction mixture was filtered and purified by prep-HPLC (column: Waters xbridge 150*25 mm*10 μm; mobile phase: [water (NH₄HCO₃)-ACN]; gradient: 50%-80% B over 10 min) to give 4-(4-(4-(2-(2-aminopyridin-3-yl)-5-(4-fluorophenyl)-3H-imidazo[4,5-b]pyridin-3-yl)benzyl)piperazin-1-yl)-1,3,5-triazine-2-carbonitrile (Example 368, 22.4 mg, yield: 5.7%) as a light-yellow solid. MS: m/z=584.4 [M+H]⁺. ¹H NMR (400 MHz, Dimethylsulfoxide-d₆) δ 8.73 (s, 1H), 8.27 (d, J=8.4 Hz, 1H), 8.08 (dd, J=8.8, 6.0 Hz, 2H), 8.01-7.97 (m, 2H), 7.53-7.50 (m, 2H), 7.48-7.45 (m, 2H), 7.33-7.28 (m, 2H), 7.15 (d, J=6.8 Hz, 1H), 7.02 (br s, 2H), 6.39 (dd, J=8.0, 4.8 Hz, 1H), 3.89-3.79 (m, 4H), 3.65 (s, 2H), 3.37-3.33 (m, 2H), 3.32-3.26 (m, 2H). ¹⁹F NMR (377 MHz, Dimethylsulfoxide-d₆) δ −113.57.

### Example 369: 4-(4-(4-(2-(2-Aminopyridin-3-yl)-5-(4-chlorophenyl)-3H-imidazo[4,5-b]pyridin-3-yl)benzyl)piperazin-1-yl)-1,3,5-triazine-2-carbonitrile

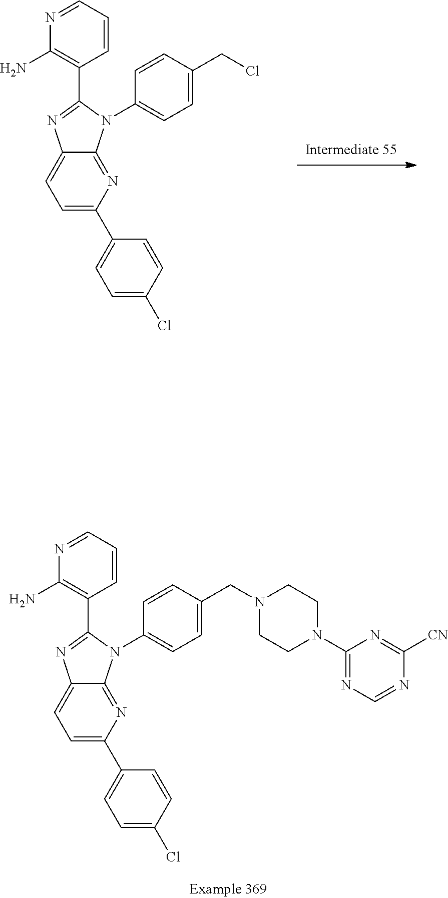 [SMILES: Nc1ncccc1-c1nc2ccc(-c3ccc(Cl)cc3)nc2n1-c1ccc(CCl)cc1 ; N#Cc1ncnc(N2CCN(Cc3ccc(-n4c(-c5cccnc5N)nc5ccc(-c6ccc(Cl)cc6)nc54)cc3)CC2)n1]

To a solution of 3-(3-(4-(chloromethyl)phenyl)-5-(4-chlorophenyl)-3H-imidazo[4,5-b]pyridin-2-yl)pyridin-2-amine (refer to Intermediate 43 for detail procedures, 300 mg, 621 μmol) and Intermediate 55 (189 mg, 621 μmol) in DMF (2 mL) were added NaI (46.4 mg, 311 μmol) and K₂CO₃ (429 mg, 3.11 mmol). The mixture was stirred at 50° C. for 2 hr. The reaction mixture was filtered and purified by prep-HPLC (column: Waters xbridge 150*25 mm*10 μm; mobile phase: [water (NH₄HCO₃)-ACN]; gradient: 58%-88% B over 10 min) to give 4-(4-(4-(2-(2-aminopyridin-3-yl)-5-(4-chlorophenyl)-3H-imidazo[4,5-b]pyridin-3-yl)benzyl)piperazin-1-yl)-1,3,5-triazine-2-carbonitrile (Example 369, 12 mg, yield: 3.1%) as a light-yellow solid. MS: m/z=600.4 [M+H]⁺. ¹H NMR (400 MHz, Dimethylsulfoxide-d₆) δ 8.73 (s, 1H), 8.29 (d, J=8.4 Hz, 1H), 8.06 (d, J=8.4 Hz, 2H), 8.01 (d, J=8.0 Hz, 2H), 7.55-7.52 (m, 4H), 7.49-7.46 (m, 2H), 7.16 (d, J=7.2 Hz, 1H), 7.03 (br s, 2H), 6.39 (dd, J=7.6, 4.8 Hz, 1H), 3.89-3.80 (m, 4H), 3.65 (s, 2H), 3.37-3.34 (m, 2H), 3.31-3.26 (m, 2H).

### Example 370: 4-((1-(4-(2-(2-Aminopyridin-3-yl)-5-(4-fluorophenyl)-3H-imidazo[4,5-b]pyridin-3-yl)benzyl)piperidin-4-yl)amino)pyrimidine-2-carbonitrile

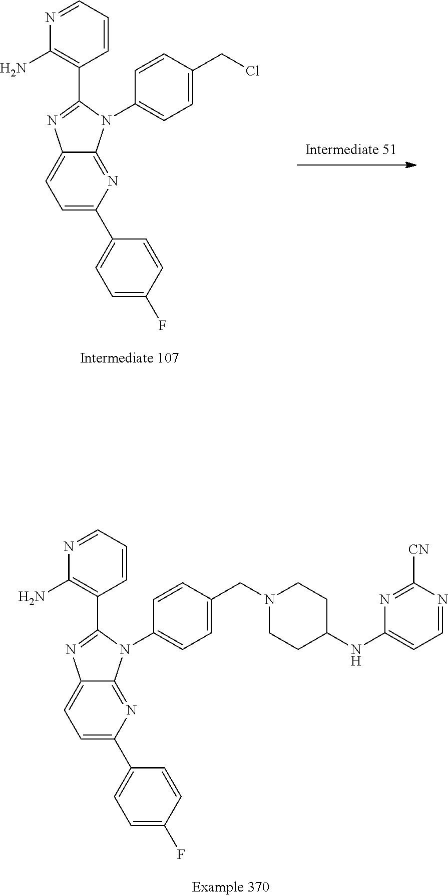 [SMILES: Nc1ncccc1-c1nc2ccc(-c3ccc(F)cc3)nc2n1-c1ccc(CCl)cc1 ; N#Cc1nccc(NC2CCN(Cc3ccc(-n4c(-c5cccnc5N)nc5ccc(-c6ccc(F)cc6)nc54)cc3)CC2)n1]

To a solution of Intermediate 107 (250 mg, 581 μmol) and Intermediate 51 (180 mg, 567 μmol) in ACN (10 mL) were added K₂CO₃ (482 mg, 3.49 mmol) and NaI (17.4 mg, 116 μmol). The mixture was stirred at 80° C. for 3 hr. The reaction mixture was diluted with H₂O (50 mL) and extracted with CH₂Cl₂ (50 mL×2). The combined organic layers were washed with brine (50 mL×2), dried over Na₂SO₄, filtered and concentrated under reduced pressure. After purified by silica gel flash chromatography (Eluent of 0˜8% MeOH in CH₂Cl₂), 4-((1-(4-(2-(2-aminopyridin-3-yl)-5-(4-fluorophenyl)-3H-imidazo[4,5-b]pyridin-3-yl)benzyl)piperidin-4-yl)amino)pyrimidine-2-carbonitrile (Example 370, 76.6 mg, yield: 22%) was obtained as a light-yellow solid. MS: m/z=597.4 [M+H]⁺. ¹H NMR (400 MHz, Dimethylsulfoxide-d₆) δ 8.27 (d, J=8.4 Hz, 1H), 8.15-8.01 (m, 4H), 8.01-7.92 (m, 2H), 7.52-7.49 (m, 4H), 7.33-7.28 (m, 2H), 7.14 (d, J=6.8 Hz, 1H), 7.03 (br s, 2H), 6.67 (d, J=6.4 Hz, 1H), 6.37 (dd, J=7.6, 4.8 Hz, 1H), 3.95-3.73 (m, 1H), 3.59 (s, 2H), 2.87-2.80 (m, 2H), 2.20-2.18 (m, 2H), 1.95-1.86 (m, 2H), 1.57-1.41 (m, 2H).

### Example 371: 2-((1-(4-(2-(2-Aminopyridin-3-yl)-5-(4-chlorophenyl)-3H-imidazo[4,5-b]pyridin-3-yl)benzyl)piperidin-4-yl)amino)pyrimidine-4-carbonitrile

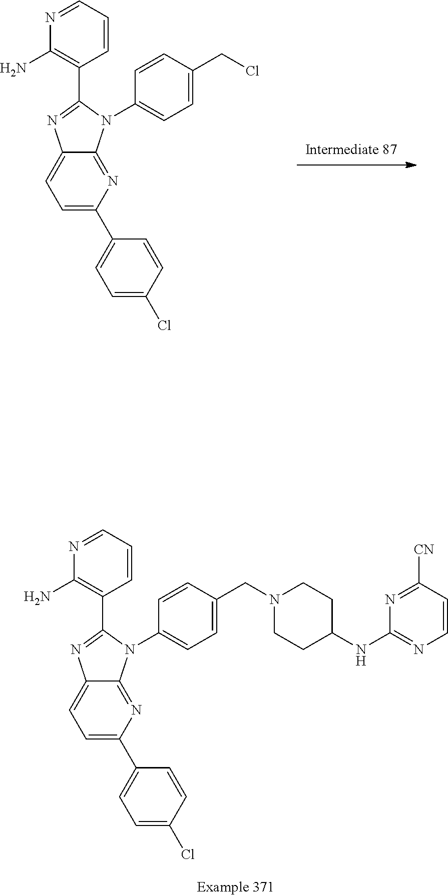 [SMILES: Nc1ncccc1-c1nc2ccc(-c3ccc(Cl)cc3)nc2n1-c1ccc(CCl)cc1 ; N#Cc1ccnc(NC2CCN(Cc3ccc(-n4c(-c5cccnc5N)nc5ccc(-c6ccc(Cl)cc6)nc54)cc3)CC2)n1]

To a solution of 3-(3-(4-(chloromethyl)phenyl)-5-(4-chlorophenyl)-3H-imidazo[4,5-b]pyridin-2-yl)pyridin-2-amine (refer to Intermediate 43 for detail procedures, 200 mg HCl salt, 414 μmol) and Intermediate 87 (131 mg TFA salt, 414 μmol) in DMF (5 mL) were added NaI (32 mg, 207 μmol) and K₂CO₃ (286 mg, 2.07 mmol). The mixture was stirred at 50° C. for 1 hr. The reaction mixture was filtered. After purified by prep-HPLC (column: Waters xbridge 150×25 mm 10 μm; mobile phase: [water (NH₄HCO₃)-ACN]; gradient: 59%-89% B over 14 min), 2-((1-(4-(2-(2-aminopyridin-3-yl)-5-(4-chlorophenyl)-3H-imidazo[4,5-b]pyridin-3-yl)benzyl)piperidin-4-yl)amino)pyrimidine-4-carbonitrile (Example 371, 48.1 mg, yield: 19%) was obtained as a yellow solid. MS: m/z=613.4 [M+H]⁺. ¹H NMR (400 MHz, Dimethylsulfoxide-d₆) δ 8.62 (d, J=4.8 Hz, 1H), 8.28 (d, J=9.2 Hz, 1H), 8.08-8.03 (m, 2H), 8.02-7.98 (m, 2H), 7.56-7.50 (m, 4H), 7.43 (d, J=8.4 Hz, 2H), 7.19 (dd, J=7.6, 1.6 Hz, 1H), 7.10 (d, J=4.8 Hz, 1H), 7.01 (br s, 2H), 6.40 (dd, J=8.0, 4.8 Hz, 1H), 4.41 (d, J=12.8 Hz, 2H), 3.86 (s, 2H), 3.18-3.11 (m, 2H), 2.79-2.70 (m, 1H), 1.95-1.86 (m, 2H), 1.32-1.22 (m, 2H).

### Example 372: 2-((1-(4-(2-(2-Aminopyridin-3-yl)-5-(3-chlorophenyl)-3H-imidazo[4,5-b]pyridin-3-yl)benzyl)piperidin-4-yl)amino)pyrimidine-4-carbonitrile

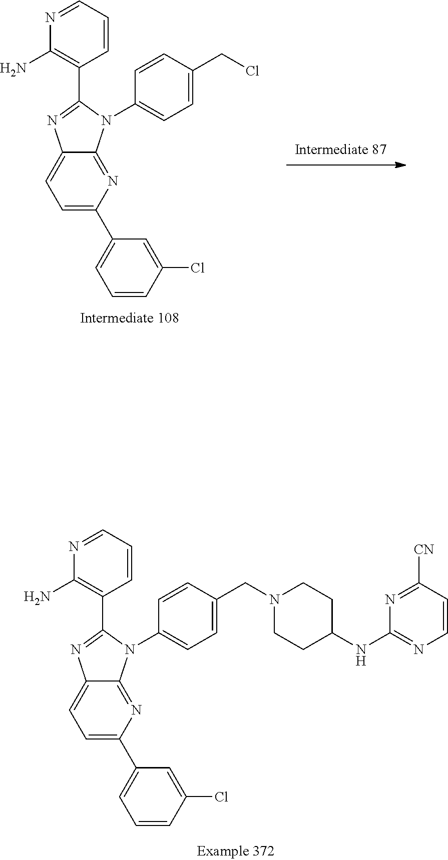 [SMILES: Nc1ncccc1-c1nc2ccc(-c3cccc(Cl)c3)nc2n1-c1ccc(CCl)cc1 ; N#Cc1ccnc(NC2CCN(Cc3ccc(-n4c(-c5cccnc5N)nc5ccc(-c6cccc(Cl)c6)nc54)cc3)CC2)n1]

To a solution of Intermediate 108 (200 mg HCl salt, 414 μmol) and Intermediate 87 (131 mg TFA salt, 414 μmol) in DMF (4 mL) were added NaI (31 mg, 207 μmol) and K₂CO₃ (286 mg, 2.07 mmol). The mixture was stirred at 50° C. for 1 hr. The reaction mixture was filtered. After purified by prep-HPLC (column: Waters xbridge 150×25 mm 10 μm; mobile phase: [water (NH₄HCO₃)-ACN]; gradient: 56%-86% B over 10 min), 2-((1-(4-(2-(2-aminopyridin-3-yl)-5-(3-chlorophenyl)-3H-imidazo[4,5-b]pyridin-3-yl)benzyl)piperidin-4-yl)amino)pyrimidine-4-carbonitrile (Example 372, 28.3 mg, yield: 11%) was obtained as a yellow solid. MS: m/z=613.3 [M+H]⁺. ¹H NMR (400 MHz, Dimethylsulfoxide-d₆) δ 8.62 (d, J=4.4 Hz, 1H), 8.29 (d, J=8.4 Hz, 1H), 8.07-8.04 (m, 2H), 8.03-7.99 (m, 2H), 7.55 (d, J=8.4 Hz, 2H), 7.53-7.40 (m, 5H), 7.22-7.18 (m, 1H), 7.10 (d, J=4.8 Hz, 1H), 7.01 (br s, 2H), 6.40 (dd, J=7.6, 4.8 Hz, 1H), 4.43-4.36 (m, 2H), 3.87 (s, 2H), 3.18-3.10 (m, 2H), 2.78-2.72 (m, 1H), 1.93-1.89 (m, 2H), 1.32-1.24 (m, 2H).

### Example 373: 6-((1-(4-(2-(2-Aminopyridin-3-yl)-5-(3-chlorophenyl)-3H-imidazo[4,5-b]pyridin-3-yl)benzyl)piperidin-4-yl)amino)pyrimidine-4-carbonitrile

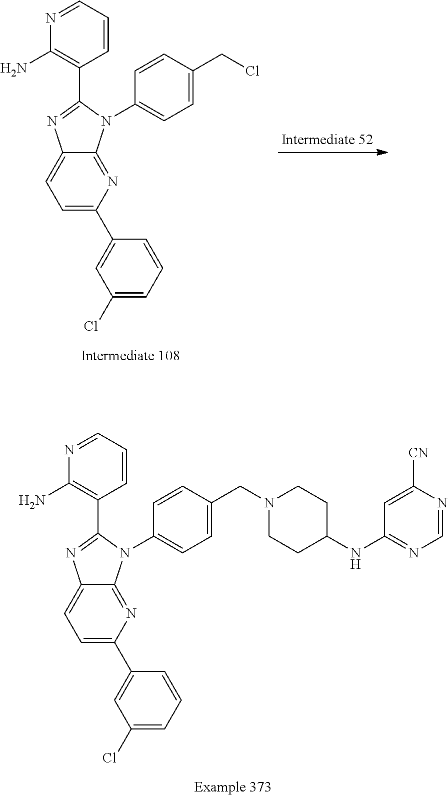 [SMILES: Nc1ncccc1-c1nc2ccc(-c3cccc(Cl)c3)nc2n1-c1ccc(CCl)cc1 ; N#Cc1cc(NC2CCN(Cc3ccc(-n4c(-c5cccnc5N)nc5ccc(-c6cccc(Cl)c6)nc54)cc3)CC2)ncn1]

To a solution of Intermediate 108 (200 mg HCl salt, 414 μmol) and Intermediate 52 (131 mg TFA salt, 414 μmol) in DMF (5 mL) were added NaI (31 mg, 207 μmol) and K₂CO₃ (286 mg, 2.07 mmol). The mixture was stirred at 50° C. for 1 hr. The reaction mixture was filtered. After purified by prep-HPLC (column: Waters xbridge 150×25 mm 10 μm; mobile phase: [water (NH₄HCO₃)-ACN]; gradient: 50%-80% B over 14 min), 6-((1-(4-(2-(2-aminopyridin-3-yl)-5-(3-chlorophenyl)-3H-imidazo[4,5-b]pyridin-3-yl)benzyl)piperidin-4-yl)amino)pyrimidine-4-carbonitrile (Example 373, 97.1 mg, yield. 38%) was obtained as a yellow solid. MS: m/z=613.4 [M+H]⁺. ¹H NMR (400 MHz, Dimethylsulfoxide-d₆) δ 8.49 (s, 1H), 8.29 (d, J=8.4 Hz, 1H), 8.08-7.98 (m, 5H), 7.52-7.44 (m, 6H), 7.17 (dd, J=8.0, 2.0 Hz, 1H), 7.04 (s, 2H), 6.92 (s, 1H), 6.38 (dd, J=8.0, 4.8 Hz, 1H), 4.00-3.76 (m, 1H), 3.59 (s, 2H), 2.87-2.80 (m, 2H), 2.16-2.09 (m, 2H), 1.93-1.82 (m, 2H), 1.56-1.45 (m, 2H).

### Example 374: 6-(4-(4-(2-(2-Aminopyridin-3-yl)-5-(3-chlorophenyl)-3H-imidazo[4,5-b]pyridin-3-yl)benzyl)piperazin-1-yl)pyrimidine-4-carbonitrile

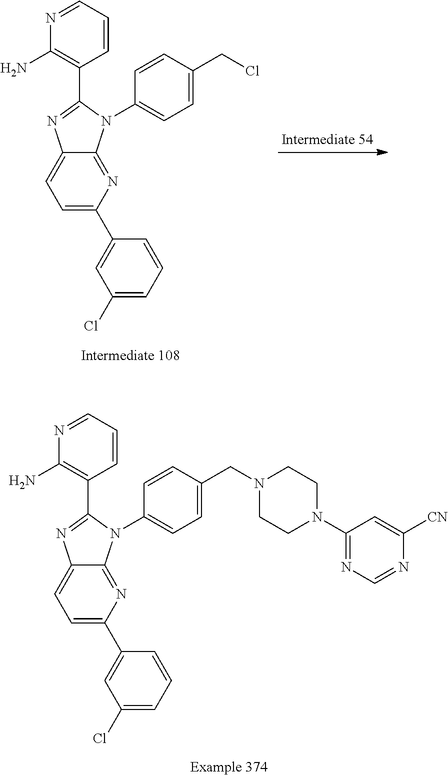 [SMILES: Nc1ncccc1-c1nc2ccc(-c3cccc(Cl)c3)nc2n1-c1ccc(CCl)cc1 ; N#Cc1cc(N2CCN(Cc3ccc(-n4c(-c5cccnc5N)nc5ccc(-c6cccc(Cl)c6)nc54)cc3)CC2)ncn1]

To a solution of Intermediate 108 (207 mg, 429 μmol) and Intermediate 54 (130 mg, 429 μmol) in DMF (3 mL) were added NaI (32.1 mg, 214 μmol) and K₂CO₃ (296 mg, 2.14 mmol). The mixture was stirred at 50° C. for 0.5 hr. The reaction mixture was filtered. After purified by prep-HPLC (column: Waters xbridge 150*25 mm*10 μm; mobile phase: [water (NH₄HCO₃)-ACN]; gradient: 54%-84% B over 14 min), 6-(4-(4-(2-(2-aminopyridin-3-yl)-5-(3-chlorophenyl)-3H-imidazo[4,5-b]pyridin-3-yl)benzyl)piperazin-1-yl)pyrimidine-4-carbonitrile (Example 374, 79.4 mg, yield: 30%) was obtained as a light-yellow solid. MS: m/z=599.4 [M+H]⁺. ¹H NMR (400 MHz, Dimethylsulfoxide-d₆) δ 8.57 (s, 1H), 8.29 (d, J=8.4 Hz, 1H), 8.07-8.04 (m, 2H), 8.03-7.99 (m, 2H), 7.58-7.56 (m, 1H), 7.54-7.50 (m, 3H), 7.49-7.46 (m, 3H), 7.16 (dd, J=7.6, 1.6 Hz, 1H), 7.05 (br s, 2H), 6.36 (dd, J=7.6, 4.8 Hz, 1H), 3.81-3.69 (m, 4H), 3.65 (s, 2H), 3.36-3.34 (m, 2H), 3.32-3.29 (m, 2H).

### Example 375: 4-((1S,4S)-5-(4-(2-(2-Aminopyridin-3-yl)-5-phenyl-3H-imidazo[4,5-b]pyridin-3-yl)benzyl)-2,5-diazabicyclo[2.2.1]heptan-2-yl)pyrimidine-2-carbonitrile

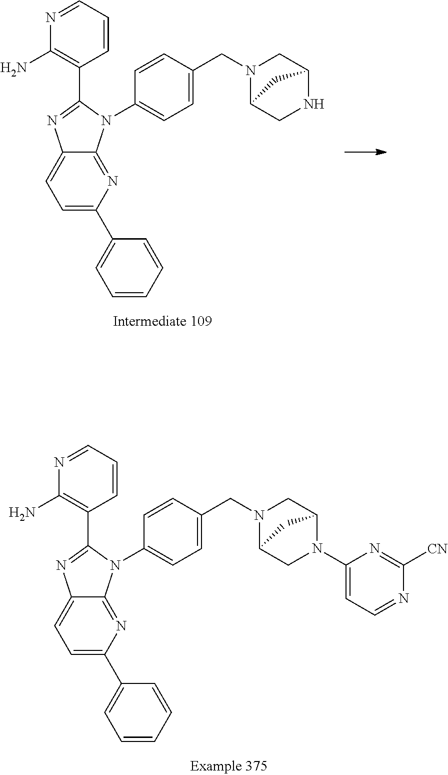

A mixture of Intermediate 109 (100 mg, 211 μmol), 4-chloropyrimidine-2-carbonitrile (29 mg, 211 μmol) and DIEA (184 μL, 1.06 mmol) in NMP (1 mL) was taken up into a microwave tube. The sealed tube was heated at 130° C. for 1 hr under microwave. The reaction mixture was filtered and concentrated under reduced pressure. The residue was purified by prep-HPLC (column: Waters xbridge 150×25 mm 10 μm; mobile phase: [water (NH₄HCO₃)-ACN]; B %: 42%-72%, 10 min) to 4-((1S,4S)-5-(4-(2-(2-aminopyridin-3-yl)-5-phenyl-3H-imidazo[4,5-b]pyridin-3-yl)benzyl)-2,5-diazabicyclo[2.2.1]heptan-2-yl)pyrimidine-2-carbonitrile (Example 375, 21.6 mg, yield: 17%) as a yellow solid. MS: m/z=577.3 [M+H]⁺. ¹H NMR (400 MHz, Dimethylsulfoxide-d₆) δ 8.26 (d, J=8.4 Hz, 1H), 8.24-8.18 (m, 1H), 8.04-7.96 (m, 4H), 7.52-7.39 (m, 7H), 7.16 (dd, J=7.6, 1.6 Hz, 1H), 7.00 (br s, 2H), 6.42-6.35 (m, 1H), 6.99-6.67 (m, 1H)), 4.94-4.65 (m, 1H), 3.84 (s, 2H), 3.71-3.53 (m, 2H), 3.44-3.38 (m, 2H), 3.00-2.89 (m, 1H), 2.06-1.95 (m, 1H), 1.88-1.76 (m, 1H).

### Example 376: 6-((1 S,4S)-5-(4-(2-(2-Aminopyridin-3-yl)-5-phenyl-3H-imidazo[4,5-b]pyridin-3-yl)benzyl)-2,5-diazabicyclo[2.2.1]heptan-2-yl)pyrimidine-4-carbonitrile

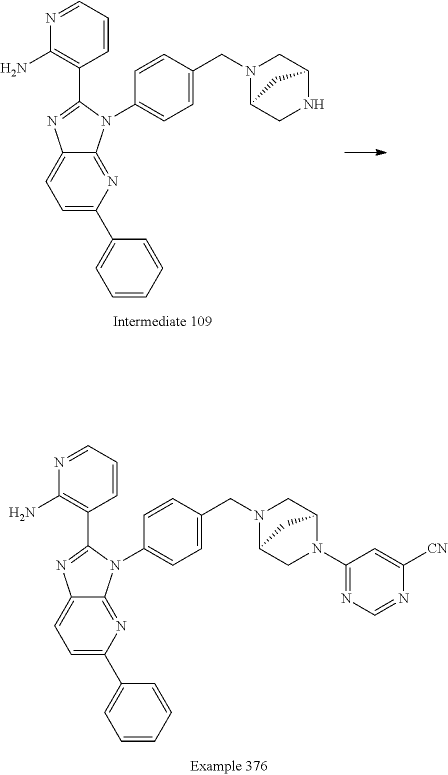

A mixture of Intermediate 109 (100 mg, 211 μmol), 6-chloropyrimidine-4-carbonitrile (29 mg, 211 μmol) and DIEA (184 μL, 1.06 mmol) in NMP (1 mL) was taken up into a microwave tube. The sealed tube was heated at 130° C. for 1 hr under microwave. The reaction mixture was filtered and concentrated under reduced pressure. The residue was purified by prep-HPLC (column: Waters xbridge 150×25 mm 10 μm; mobile phase: [water (NH₄HCO₃)-ACN]; B %: 44%-74%, 10 min) to give 6-((1S,4S)-5-(4-(2-(2-aminopyridin-3-yl)-5-phenyl-3H-imidazo[4,5-b]pyridin-3-yl)benzyl)-2,5-diazabicyclo[2.2.1]heptan-2-yl)pyrimidine-4-carbonitrile (Example 376, 24.7 mg, yield: 20%) as a yellow solid. MS: m/z=577.3 [M+H]⁺. ¹H NMR (400 MHz, Dimethylsulfoxide-d₆) δ 8.52 (d, J=12.8 Hz, 1H), 8.26 (d, J=8.0 Hz, 1H), 8.04-7.97 (m, 4H), 7.53-7.37 (m, 8H), 7.17-7.15 (m, 1H), 7.00 (br s, 2H), 6.39 (dd, J=8.0, 4.8 Hz, 1H), 5.00-4.67 (m, 1H), 3.86-3.79 (m, 2H), 3.72-3.57 (m, 2H), 3.45-3.36 (m, 2H), 2.96-2.89 (m, 1H), 2.05-1.96 (m, 1H), 1.87-1.78 (m, 1H).

### Example 377: 2-((1 S,4S)-5-(4-(2-(2-Aminopyridin-3-yl)-5-phenyl-3H-imidazo[4,5-b]pyridin-3-yl)benzyl)-2,5-diazabicyclo[2.2.1]heptan-2-yl)pyrimidine-4-carbonitrile

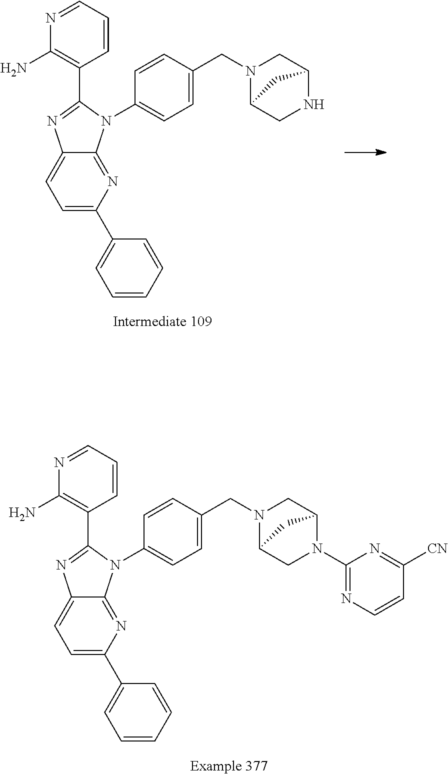

A mixture of Intermediate 109 (100 mg, 211 μmol), 2-chloropyrimidine-4-carbonitrile (29 mg, 211 μmol) and DIEA (184 μL, 1.06 mmol) in NMP (1 mL) was taken up into a microwave tube. The sealed tube was heated at 130° C. for 1 hr under microwave. The reaction mixture was filtered and concentrated under reduced pressure. The residue was purified by prep-HPLC (column: Waters xbridge 150×25 mm 10 μm; mobile phase: [water (NH₄HCO₃)-ACN]; B %: 50%-80%, 10 min) to give 2-((1S,4S)-5-(4-(2-(2-aminopyridin-3-yl)-5-phenyl-3H-imidazo[4,5-b]pyridin-3-yl)benzyl)-2,5-diazabicyclo[2.2.1]heptan-2-yl)pyrimidine-4-carbonitlile (Example 377, 13.1 mg, yield: 10%) as a yellow solid. MS: m/z=577.3 [M+H]⁺. ¹H NMR (400 MHz, Dimethylsulfoxide-d₆) δ 8.61 (dd, J=13.2, 4.8 Hz, 1H), 8.26 (d, J=8.0 Hz, 1H), 8.04-7.96 (m, 4H), 7.52-7.49 (m, 2H), 7.47-7.43 (m, 3H), 7.42-7.37 (m, 2H), 7.16 (dd, J=7.6, 1.6 Hz, 1H), 7.13 (d, J=4.8 Hz, 1H), 7.01 (br s, 2H), 6.39 (dd, J=7.2, 4.4 Hz, 1H), 4.84-4.70 (m, 1H), 3.83 (s, 2H), 3.70-3.64 (m, 2H), 3.45-3.35 (m, 2H), 2.98-2.93 (m, 1H), 2.59-2.52 (m, 2H), 2.00-1.96 (m, 1H), 1.85-1.81 (m, 1H).

### Example 378: 4-((2S,6R)-4-(4-(2-(2-Aminopyridin-3-yl)-5-phenyl-3H-imidazo[4,5-b]pyridin-3-yl)benzyl)-2,6-dimethylpiperazin-1-yl)pyrimidine-2-carbonitrile

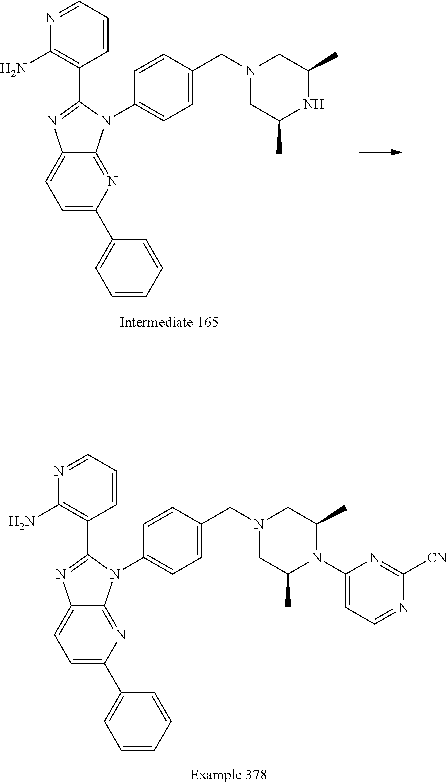 [SMILES: C[C@@H]1CN(Cc2ccc(-n3c(-c4cccnc4N)nc4ccc(-c5ccccc5)nc43)cc2)C[C@H](C)N1 ; C[C@@H]1CN(Cc2ccc(-n3c(-c4cccnc4N)nc4ccc(-c5ccccc5)nc43)cc2)C[C@H](C)N1c1ccnc(C#N)n1]

A mixture of Intermediate 165 (80 mg, 164 μmol), 4-chloropyrimidine-2-carbonitrile (22.8 mg, 164 μmol) and DIEA (106 mg, 817 μmol) in NMP (2 mL) was taken up into a microwave tube. The sealed tube was heated at 160° C. for 1 hr under microwave. The reaction mixture was filtered to give a filter liquor. The filter liquor was purified byprep-HPLC (column: Welch Xtimate C18 150×25 mm×5 μm; mobile phase: [water (NH₄HCO₃)-ACN]; B %: 57%-87%, 10 min) to give 4-((2S,6R)-4-(4-(2-(2-aminopyridin-3-yl)-5-phenyl-3H-imidazo[4,5-b]pyridin-3-yl)benzyl)-2,6-dimethylpiperazin-1-yl)pyrimidine-2-carbonitrile (Example 378, 6.4 mg, yield: 6.6%) as a gray solid. MS: m/z=593.4 [M+H]⁺. ¹H NMR (400 MHz, Methanol-d₄) 8.21-8.18 (m, 2H), 8.06-8.04 (m, 2H), 8.00-7.98 (m, 1H), 7.95 (d, J=8.4 Hz, 1H), 7.62 (d, J=8.0 Hz, 2H), 7.48-7.41 (m, 4H), 7.39-7.37 (m, 1H), 7.35 (dd, J=7.2, 1.6 Hz, 1H), 6.88 (d, J=6.4 Hz, 1H), 6.48 (dd, J=7.6, 4.8 Hz, 1H), 3.67 (s, 2H), 3.30-3.27 (m, 2H), 2.89-2.87 (m, 1H), 2.89-2.85 (m, 1H), 2.39-2.31 (m, 2H), 1.26 (d, J=6.0 Hz, 6H).

### Example 379: 2-((2S,6R)-4-(4-(2-(2-Aminopyridin-3-yl)-5-phenyl-3H-imidazo[4,5-b]pyridin-3-yl)benzyl)-2,6-dimethylpiperazin-1-yl)pyrimidine-4-carbonitrile

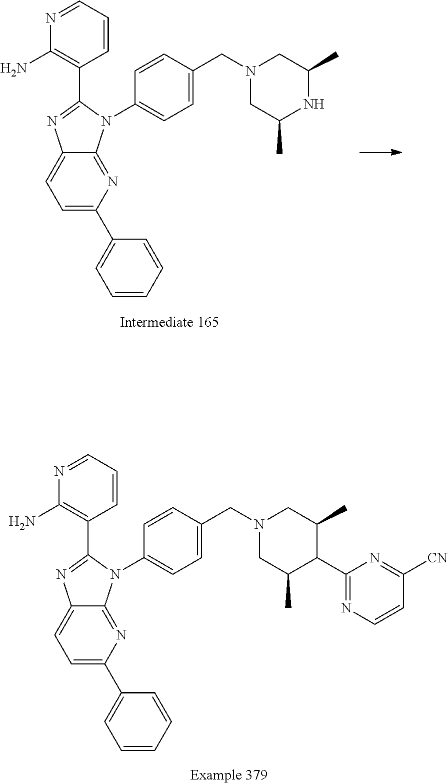 [SMILES: C[C@@H]1CN(Cc2ccc(-n3c(-c4cccnc4N)nc4ccc(-c5ccccc5)nc43)cc2)C[C@H](C)N1 ; C[C@@H]1CN(Cc2ccc(-n3c(-c4cccnc4N)nc4ccc(-c5ccccc5)nc43)cc2)C[C@H](C)C1c1nccc(C#N)n1]

A mixture of Intermediate 165 (80 mg, 164 μmol), 2-chloropyrimidine-4-carbonitrile (22.8 mg, 164 μmol) and DIEA (106 mg, 817 μmol) in NMP (2 mL) was taken up into a microwave tube. The sealed tube was heated at 160° C. for 1 hr under microwave. The reaction mixture was filtered to give a filter liquor. The filter liquor was purified by prep-HPLC (column: Welch Xtimate C18 150×25 mm×5 μm; mobile phase: [water (NH₄HCO₃)-ACN]; B %. 66%-96%, 10 min) to give 2-((2S,6R)-4-(4-(2-(2-aminopyridin-3-yl)-5-phenyl-3H-imidazo[4,5-b]pyridin-3-yl)benzyl)-2,6-dimethylpiperazin-1-yl)pyrimidine-4-carbonitrile (Example 379, 3.7 mg, yield: 3.7%) as a brown lyophilized powder. MS: m/z=593.2 [M+H]⁺. ¹H NMR (400 MHz, Dimethylsulfoxide-d₆) 8.67 (d, J=4.8 Hz, 1H), 8.27 (d, J 8.4 Hz, 1H), 8.06-8.03 (m, 2H), 8.02-7.98 (m, 2H), 7.60 (d, J=8.4 Hz, 2H), 7.49 (d, J=8.4 Hz, 2H), 7.46-7.43 (m, 2H), 7.41-7.38 (m, 1H), 7.19-7.15 (m, 2H), 7.06 (br s, 2H), 6.40 (dd, J=7.6, 4.8 Hz, 1H), 4.60-4.59 (m, 2H), 3.65 (s, 2H), 2.78 (d, J=11.2 Hz, 2H), 2.27-2.23 (m, 2H), 1.28 (d, J=6.4 Hz, 6H).

### Example 380: 6-((2S,6R)-4-(4-(2-(2-Aminopyridin-3-yl)-5-phenyl-3H-imidazo[4,5-b]pyridin-3-yl)benzyl)-2,6-dimethylpiperazin-1-yl)pyrimidine-4-carbonitrile

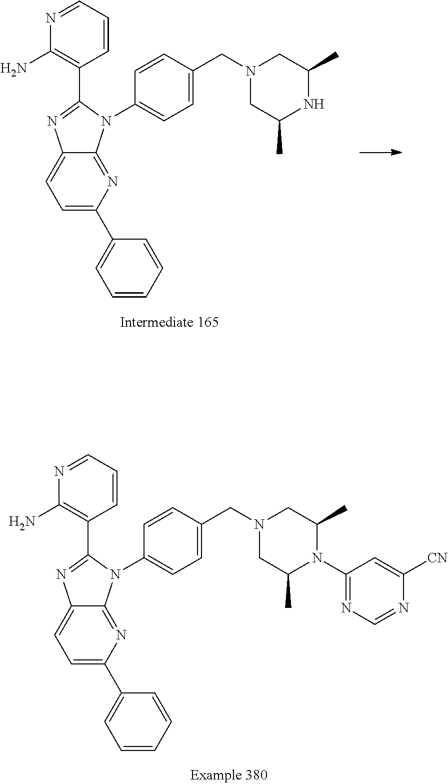 [SMILES: C[C@@H]1CN(Cc2ccc(-n3c(-c4cccnc4N)nc4ccc(-c5ccccc5)nc43)cc2)C[C@H](C)N1 ; C[C@@H]1CN(Cc2ccc(-n3c(-c4cccnc4N)nc4ccc(-c5ccccc5)nc43)cc2)C[C@H](C)N1c1cc(C#N)ncn1]

A mixture of Intermediate 165 (80 mg, 164 μmol), 6-chloropyrimidine-4-carbonitrile (22.8 mg, 164 μmol) and DIEA (106 mg, 817 μmol) in NMP (2 mL) was taken up into a microwave tube. The sealed tube was heated at 160° C. for 1 hr under microwave. The reaction mixture was filtered to give a filter liquor. The filter liquor was purified by prep-HPLC (column: Welch Xtimate C18 150×25 mm×5 μm; mobile phase: [water (NH₄HCO₃)-ACN]; B %: 55%-85%, 10 min) to 6-((2S,6R)-4-(4-(2-(2-Aminopyridin-3-yl)-5-phenyl-3H-imidazo[4,5-b]pyridin-3-yl)benzyl)-2,6-dimethylpiperazin-1-yl)pyrimidine-4-carbonitrile (Example 380, 8.5 mg, yield: 8.7%) as a brown lyophilized powder. MS: m/z=593.4 [M+H]⁺. ¹H NMR (400 MHz, Methanol-d₄) δ 8.54 (s, 1H), 8.20 (d, J=8.4 Hz, 1H), 8.05 (d, J=7.2 Hz, 2H), 7.99 (dd, J=5.2, 1.6 Hz, 1H), 7.95 (d, J=8.4 Hz, 1H), 7.62 (d, J=8.4 Hz, 2H), 7.43-7.41 (m, 4H), 7.39-7.37 (m, 1H), 7.34 (dd, J=7.6, 1.6 Hz, 1H), 7.23 (s, 1H), 6.49 (dd, J=7.6, 4.8 Hz, 1H), 3.69 (s, 2H), 3.34-3.30 (m, 2H), 2.86 (d, J 11.2 Hz, 2H), 2.33 (d, J 11.2 Hz, 2H), 1.37 (d, J=6.4 Hz, 6H).

### Example 381: 4-((1-(4-(2-(2-Aminopyridin-3-yl)-5-(2-oxo-1,2-dihydropyridin-3-yl)-3H-imidazo[4,5-b]pyridin-3-yl)benzyl)piperidin-4-yl)amino)pyrimidine-2-carbonitrile

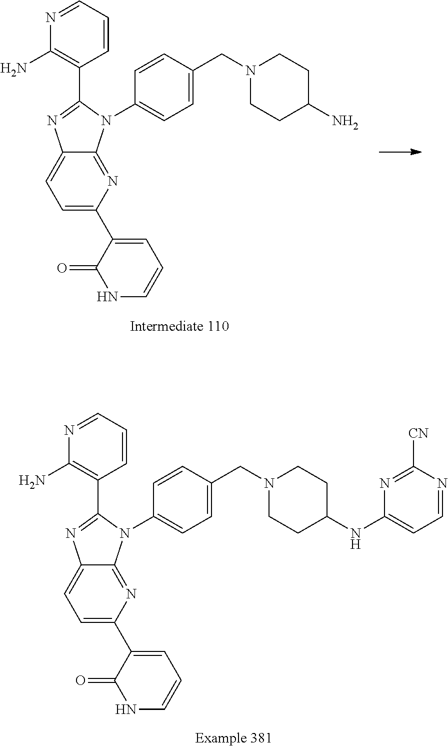 [SMILES: Nc1ncccc1-c1nc2ccc(-c3ccc[nH]c3=O)nc2n1-c1ccc(CN2CCC(N)CC2)cc1 ; N#Cc1nccc(NC2CCN(Cc3ccc(-n4c(-c5cccnc5N)nc5ccc(-c6ccc[nH]c6=O)nc54)cc3)CC2)n1]

A mixture of Intermediate 110 (85 mg, 173 μmol), 4-chloropyrimidine-2-carbonitrile (26.5 mg, 190 μmol) and DIEA (67 mg, 518 μmol) in NMP (2 mL) was taken up into a microwave tube. The sealed tube was heated at 130° C. for 30 min under microwave. The reaction mixture was filtered. The residue was purified by prep-HPLC (column: Waters xbridge 150*25 mm 10 μm; mobile phase: [water (NH₄HCO₃)-ACN]; B %: 6%-46%, 14 min) to give 4-((1-(4-(2-(2-aminopyridin-3-yl)-5-(2-oxo-1,2-dihydropyridin-3-yl)-3H-imidazo[4,5-b]pyridin-3-yl)benzyl)piperidin-4-yl)amino)pyrimidine-2-carbonitrile (Example 381, 10.2 mg, yield: 9.3%) as a light yellow solid. MS: m/z=596.3 [M+H]⁺. ¹H NMR (400 MHz, Dimethylsulfoxide-d₆) δ 11.98 (br s, 1H), 8.68 (d, J=8.4 Hz, 1H), 8.23 (dd, J=7.2, 2.0 Hz, 1H), 8.18 (d, J=8.4 Hz, 1H), 8.12-7.01 (m, 2H), 8.01-7.97 (m, 1H), 7.49-7.41 (m, 5H), 7.13 (d, J=7.6 Hz, 1H), 7.04 (br s, 2H), 6.67 (d, J=6.0 Hz, 1H), 6.39-6.34 (m, 2H), 3.90-3.75 (m, 1H), 3.59 (s, 2H), 2.86-2.80 (m, 2H), 2.20-2.10 (m, 2H), 1.94-1.84 (m, 2H), 1.56-1.45 (m, 2H).

### Example 382: 4-((1-(4-(2-(2-Aminopyridin-3-yl)-5-(4-chlorophenyl)-3H-imidazo[4,5-b]pyridin-3-yl)benzyl)piperidin-4-yl)amino)-1,3,5-triazine-2-carbonitrile

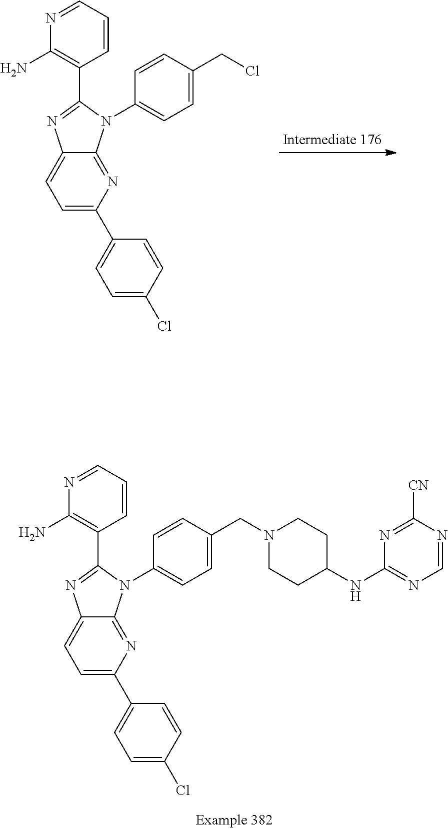 [SMILES: Nc1ncccc1-c1nc2ccc(-c3ccc(Cl)cc3)nc2n1-c1ccc(CCl)cc1 ; N#Cc1ncnc(NC2CCN(Cc3ccc(-n4c(-c5cccnc5N)nc5ccc(-c6ccc(Cl)cc6)nc54)cc3)CC2)n1]

A mixture of 3-(3-(4-(chloromethyl)phenyl)-5-(4-chlorophenyl)-3H-imidazo[4,5-b]pyridin-2-yl)pyridin-2-amine (refer to Intermediate 43 for detail procedures, 300 mg, 621 μmol), Intermediate 175 (198 mg, 621 μmol), NaI (47 mg, 311 μmol), and K₂CO₃ (429 mg, 3 mmol) in DMF (5 mL) was degassed and purged with N₂ three times, and then the mixture was stirred at 50° C. for 1 hr under N₂ atmosphere. After purified by prep-HPLC (column: Phenomenex C18 250×50 mm×10 μm; mobile phase: [water (NH₄HCO₃)-ACN]; gradient: 47%-77% B over 11 min), 4-((1-(4-(2-(2-aminopyridin-3-yl)-5-(4-chlorophenyl)-3H-imidazo[4,5-b]pyridin-3-yl)benzyl)piperidin-4-yl)amino)-1,3,5-triazine-2-carbonitrile (Example 382, 22.8 mg, yield: 5.6%) was obtained as yellow solid. MS: m/z=614.4 [M+H]⁺. ¹H NMR (400 MHz, Dimethyl sulfoxide-d₆) δ 8.97-8.90 (m, 1H), 8.68-8.61 (m, 1H), 8.28 (d, J=8.4 Hz, 1H), 8.09-7.97 (m, 4H), 7.53 (d, J=8.4 Hz, 2H), 7.50-7.43 (m, 4H), 7.15 (d, J=7.6 Hz, 1H), 7.03 (br s, 2H), 6.36-6.35 (m, 1H), 3.84-3.69 (m, 1H), 3.57 (s, 2H), 2.89-2.81 (m, 2H), 2.15-2.02 (m, 2H), 1.86-1.80 (m, 2H), 1.64-1.50 (m, 2H).

### Example 383: 4-(4-(4-(2-(2-Aminopyridin-3-yl)-5-(4-cyanopyridin-3-yl)-3H-imidazo[4,5-b]pyridin-3-yl)benzyl)piperazin-1-yl)pyrimidine-2-carbonitrile

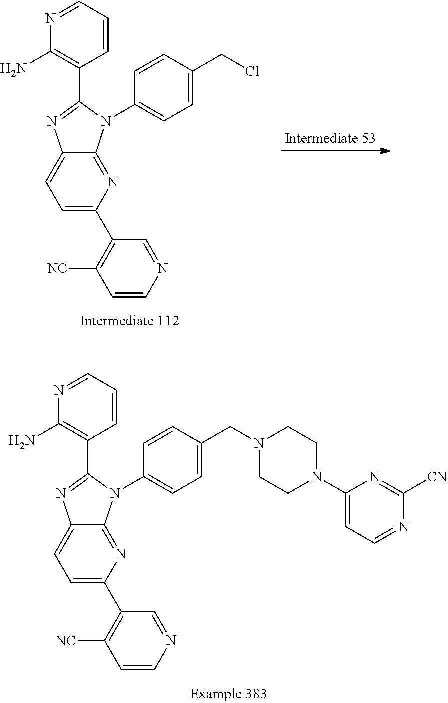 [SMILES: N#Cc1ccncc1-c1ccc2nc(-c3cccnc3N)n(-c3ccc(CCl)cc3)c2n1 ; N#Cc1nccc(N2CCN(Cc3ccc(-n4c(-c5cccnc5N)nc5ccc(-c6cnccc6C#N)nc54)cc3)CC2)n1]

To a solution of Intermediate 112 (100 mg, 228 μmol) and Intermediate 53 (43 mg, 228 μmol) in DMF (5 mL) were added NaI (17 mg, 114 μmol) and K₂CO₃ (158 mg, 1.0 mmol). The mixture was stirred at 50° C. for 1 hr. The reaction mixture was diluted with water (20 mL) and extracted with EtOAc (50 mL×2). The combined organic layers were washed with brine (30 mL), dried over Na₂SO₄, filtered and concentrated under reduced pressure. After purified by silica gel flash chromatography (Eluent of 0˜7% MeOH in CH₂Cl₂), 4-(4-(4-(2-(2-aminopyridin-3-yl)-5-(4-cyanopyridin-3-yl)-3H-imidazo[4,5-b]pyridin-3-yl)benzyl)piperazin-1-yl)pyrimidine-2-carbonitrile (Example 383, 40.9 mg, yield: 30%) was obtained as a yellow solid. MS: m/z=591.4 [M+H]⁺. ¹H NMR (400 MHz, Dimethylsulfoxide-d₆) δ 9.20 (s, 1H), 8.84 (d, J=5.2 Hz, 1H), 8.43 (d, J=8.4 Hz, 1H), 8.26 (d, J=6.4 Hz, 1H), 8.04-7.96 (m, 3H), 7.51-7.45 (m, 4H), 7.21 (d, J=6.0 Hz, 1H), 7.11 (d, J=6.4 Hz, 1H), 7.01 (br s, 2H), 6.40 (dd, J=7.6, 5.2 Hz, 1H), 3.76-3.63 (m, 4H), 3.61 (s, 2H), 2.48-2.43 (m, 4H).

### Example 384: 4-((1-(4-(2-(2-Aminopyridin-3-yl)-5-(4-cyanopyridin-3-yl)-3H-imidazo[4,5-b]pyridin-3-yl)benzyl)piperidin-4-yl)amino)pyrimidine-2-carbonitrile

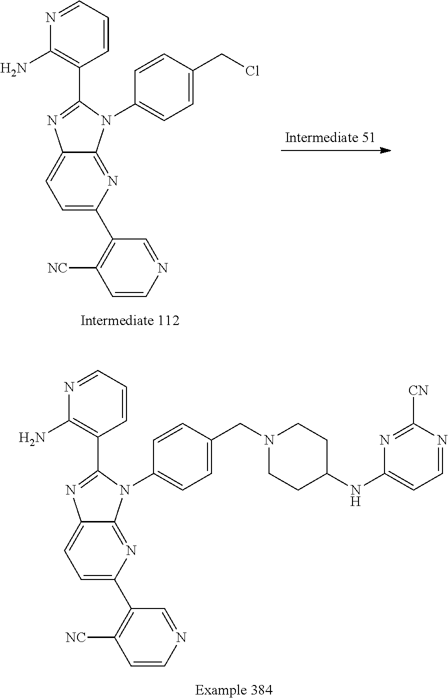 [SMILES: N#Cc1ccncc1-c1ccc2nc(-c3cccnc3N)n(-c3ccc(CCl)cc3)c2n1 ; N#Cc1nccc(NC2CCN(Cc3ccc(-n4c(-c5cccnc5N)nc5ccc(-c6cnccc6C#N)nc54)cc3)CC2)n1]

To a solution of Intermediate 112 (100 mg, 228 μmol) and Intermediate 51 (72 mg, 357 μmol) in DMF (5 mL) were added NaI (17 mg, 114 μmol) and K₂CO₃ (158 mg, 1.0 mmol). The mixture was stirred at 50° C. for 1 hr. The reaction mixture was diluted with water (20 mL) and extracted with EtOAc (50 mL×2). The combined organic layers were washed with brine (30 mL), dried over Na₂SO₄, filtered and concentrated under reduced pressure. After purified by silica gel flash chromatography (Eluent of 0˜7% MeOH in CH₂Cl₂), 4-((1-(4-(2-(2-aminopyridin-3-yl)-5-(4-cyanopyridin-3-yl)-3H-imidazo[4,5-b]pyridin-3-yl)benzyl)piperidin-4-yl)amino)pyrimidine-2-carbonitrile (Example 384, 24.2 mg, yield: 18%) was obtained as a yellow solid. MS: m/z=605.4 [M+H]⁺. ¹H NMR (400 MHz, Dimethylsulfoxide-d₆) δ 9.25-9.24 (m, 1H), 8.84 (d, J=4.8 Hz, 1H), 8.43 (d, J=8.0 Hz, 1H), 8.09-8.02 (m, 3H), 7.97 (d, J=4.8 Hz, 1H), 7.65 (s, 1H), 7.51-7.41 (m, 4H), 7.25-7.19 (m, 1H), 7.01 (br s, 2H), 6.67-6.66 (m, 1H), 6.44-6.38 (m, 1H), 4.06-3.69 (m, 1H), 3.55 (s, 2H), 2.82-2.80 (m, 2H), 2.15-2.08 (m, 2H), 1.92-1.81 (m, 2H), 1.52-1.42 (m, 2H).

### Example 385: 4-((1-((4-(2-(2-Aminopyridin-3-yl)-3H-imidazo[4,5-b]pyridin-3-yl)phenyl)methyl-d)piperidin-4-yl)amino)pyrimidine-2-carbonitrile

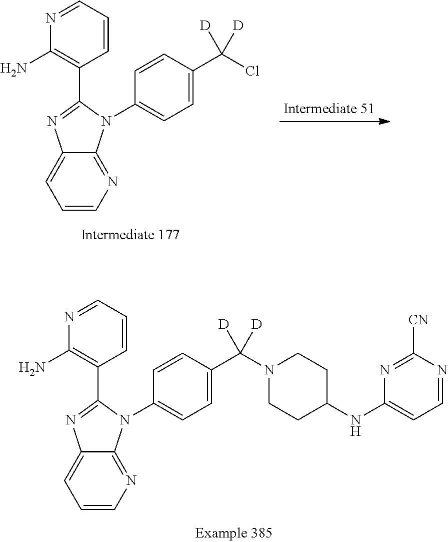 [SMILES: [2H]C([2H])(Cl)c1ccc(-n2c(-c3cccnc3N)nc3cccnc32)cc1 ; [2H]C([2H])(c1ccc(-n2c(-c3cccnc3N)nc3cccnc32)cc1)N1CCC(Nc2ccnc(C#N)n2)CC1]

To a solution of Intermediate 177 (90 mg, 266 μmol) and Intermediate 51 (81 mg, 399 mol) in DMF (2 mL) were added NaI (4.0 mg, 27 μmol) and K₂CO₃ (110 mg, 799 μmol). The mixture was stirred at 80° C. for 2 hr. The mixture was diluted with H₂O (10 mL) and extracted with EtOAc (10 mL×2). The combined organic layers were washed with brine (10 mL), dried over Na₂SO₄, filtered and concentrated under reduced pressure to give a residue. After purified by silica gel flash chromatography (Eluent of 0˜7% MeOH in CH₂Cl₂), 4-((1-((4-(2-(2-aminopyridin-3-yl)-3H-imidazo[4,5-b]pyridin-3-yl)phenyl)methyl-d)piperidin-4-yl)amino)pyrimidine-2-carbonitrile (Example 385, 32.4 mg, yield: 24%) was obtained as a light yellow solid. MS: m/z=505.2 [M+H]⁺. ¹H NMR (400 MHz, Dimethylsulfoxide-d₆) δ 8.33 (d, J=4.4 Hz, 1H), 8.21 (d, J=8.0 Hz, 1H), 8.11-8.03 (m, 2H), 7.99 (d, J=4.4 Hz, 1H), 7.47-7.43 (m, 2H), 7.41-7.37 (m, 3H), 7.16 (d, J=8.0 Hz, 1H), 7.02 (br s, 2H), 6.67 (d, J=6.0 Hz, 1H), 6.37 (dd, J=7.6, 4.8 Hz, 1H), 3.87-3.74 (m, 1H), 2.85-2.78 (m, 2H), 2.20-2.09 (m, 2H), 1.93-1.83 (m, 2H), 1.53-1.43 (m, 2H)

### Example 386: 4-(4-(4-(2-(2-Aminopyridin-3-yl)-5-(4-methyl-3-oxo-1,4-diazepan-1-yl)-3H-imidazo[4,5-b]pyridin-3-yl)benzyl)piperazin-1-yl)pyrimidine-2-carbonitrile

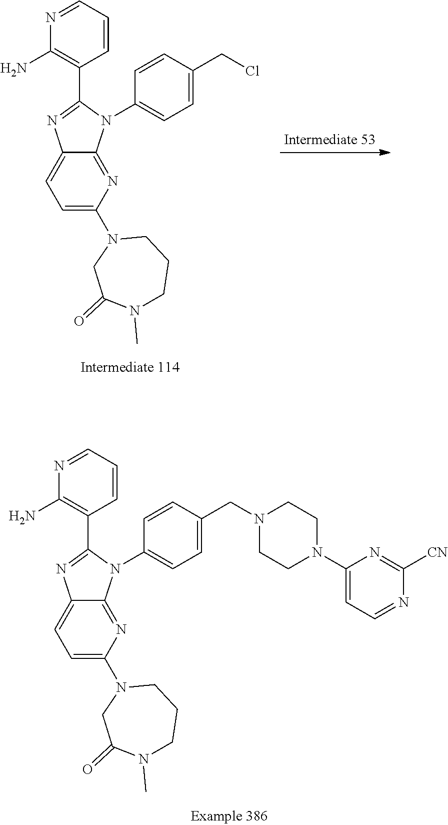 [SMILES: CN1CCCN(c2ccc3nc(-c4cccnc4N)n(-c4ccc(CCl)cc4)c3n2)CC1=O ; CN1CCCN(c2ccc3nc(-c4cccnc4N)n(-c4ccc(CN5CCN(c6ccnc(C#N)n6)CC5)cc4)c3n2)CC1=O]

To a solution of Intermediate 114 (250 mg, 541 μmol) and Intermediate 53 (113 mg, 595 μmol) in DMF (3 mL) was added DIEA (280 mg, 2.16 mmol). The mixture was stirred at 80° C. for 2 hr under N₂. The mixture was concentrated. After purified by prep-HPLC (column: Phenomenex C18 80*40 mm*3 μm; mobile phase: [water (NH₃H₂O+NH₄HCO₃)-ACN]; gradient: 42%-72% B over 7 min), 4-(4-(4-(2-(2-aminopyridin-3-yl)-5-(4-methyl-3-oxo-1,4-diazepan-1-yl)-3H-imidazo[4,5-b]pyridin-3-yl)benzyl)piperazin-1-yl)pyrimidine-2-carbonitrile (Example 386, 9 mg, yield. 2.7%) was obtained as a yellow solid. MS: m/z=615.3 [M+H]⁺. ¹H NMR (400 MHz, Dimethylsulfoxide-d₆) δ 8.27 (d, J=6.4 Hz, 1H), 8.00-7.90 (m, 2H), 7.44 (d, J=8.4 Hz, 2H), 7.34 (d, J=8.4 Hz, 2H), 7.11 (d, J=6.4 Hz, 1H), 7.05-6.92 (m, 3H), 6.80 (d, J=8.8 Hz, 1H), 6.30 (dd, J=7.6, 4.8 Hz, 1H), 4.27 (s, 2H), 3.83-3.77 (m, 2H), 3.75-3.65 (m, 6H), 3.62 (s, 2H), 3.48-3.43 (m, 2H), 2.76 (s, 3H), 2.46-2.44 (m, 2H), 1.74-1.67 (m, 2H).

### Example 387: 4-((1-(4-(2-(2-Aminopyridin-3-yl)-5-(3-oxomorpholino)-3H-imidazo[4,5-b]pyridin-3-yl)benzyl)piperidin-4-yl)amino)pyrimidine-2-carbonitrile

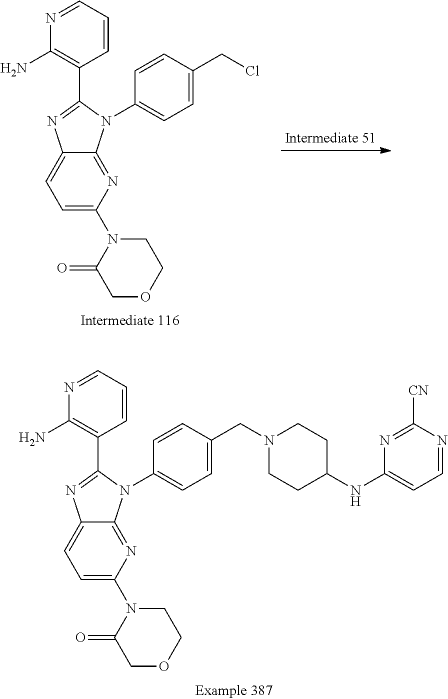 [SMILES: Nc1ncccc1-c1nc2ccc(N3CCOCC3=O)nc2n1-c1ccc(CCl)cc1 ; N#Cc1nccc(NC2CCN(Cc3ccc(-n4c(-c5cccnc5N)nc5ccc(N6CCOCC6=O)nc54)cc3)CC2)n1]

To a solution of Intermediate 116 (156 mg HCl salt, 331 μmol) and Intermediate 51 (105 mg TFA salt, 331 μmol) in DMF (2 mL) were added K₂CO₃ (137 mg, 993 μmol) and NaI (50 mg, 331 μmol). The mixture was stirred at 25° C. for 1 hr. The reaction mixture was diluted with H₂O (40 mL) and extracted with EtOAc (30 mL×2). The combined organic layers were washed with brine (30 mL), dried over Na₂SO₄, filtered and concentrated under reduced pressure. After purified by silica gel flash chromatography (Eluent of 0˜7%, MeOH in CH₂Cl₂), 4-((1-(4-(2-(2-aminopyridin-3-yl)-5-(3-oxomorpholino)-3H-imidazo[4,5-b]pyridin-3-yl)benzyl)piperidin-4-yl)amino)pyrimidine-2-carbonitrile (Example 387, 26.5 mg, yield: 12%) was obtained as a yellow solid. MS: m/z=602.4 [M+H]⁺. ¹H NMR (400 MHz, Dimethylsulfoxide-d₆) δ 8.24 (d, J=8.4 Hz, 1H), 8.12-8.03 (m, 2H), 7.98 (dd, J=4.8, 1.6 Hz, 1H), 7.84 (d, J=8.8 Hz, 1H), 7.48-7.42 (m, 2H), 7.41-7.36 (m, 2H), 7.12 (dd, J=7.6, 1.6 Hz, 1H), 6.95 (br s, 2H), 6.67 (d, J=6.0 Hz, 1H), 6.37 (dd, J=7.6, 4.8 Hz, 1H), 4.25 (s, 2H), 3.98-3.92 (m, 2H), 3.86-3.83 (m, 2H), 3.57 (s, 2H), 3.18-3.13 (m, 1H), 2.82-2.79 (m, 2H), 2.16-2.11 (m, 2H), 1.92-1.84 (m, 2H), 1.55-1.47 (m, 2H).

### Example 388: 4-((1-(4-(2-(2-Aminopyridin-3-yl)-5-cyclopentyl-3H-imidazo[4,5-b]pyridin-3-yl)benzyl)piperidin-4-yl)amino)pyrimidine-2-carbonitrile

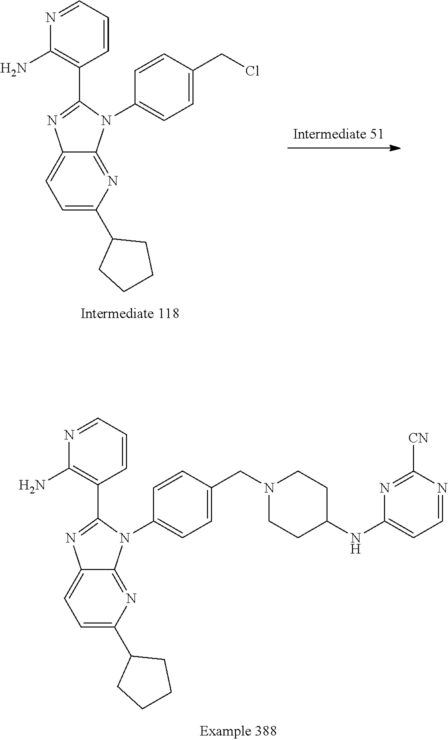 [SMILES: Nc1ncccc1-c1nc2ccc(C3CCCC3)nc2n1-c1ccc(CCl)cc1 ; N#Cc1nccc(NC2CCN(Cc3ccc(-n4c(-c5cccnc5N)nc5ccc(C6CCCC6)nc54)cc3)CC2)n1]

To a solution of Intermediate 118 (100 mg, 248 μmol) and Intermediate 51 (86.4 mg, 272 μmol) in DMF (1 mL) were added NaI (11 mg, 74 μmol) and K₂CO₃ (171 mg, 1.2 mmol). The mixture was stirred at 50° C. for 0.5 hr. The reaction mixture was poured into H₂O (15 mL), extracted with EtOAc (15 mL×3). The combined organic layers were dried over anhydrous Na₂SO₄, filtered and concentrated under reduced pressure. After purified by silica gel flash chromatography (Eluent of 0˜15% MeOH in CH₂Cl₂), 4-((1-(4-(2-(2-aminopyridin-3-yl)-5-cyclopentyl-3H-imidazo[4,5-b]pyridin-3-yl)benzyl)piperidin-4-yl)amino)pyrimidine-2-carbonitrile (Example 388, 38.3 mg, yield: 27%) was obtained as a yellow solid. MS: m/z=571.2 [M+H]⁺. ¹H NMR (400 MHz, Dimethylsulfoxide-d₆) δ 8.13-8.03 (m, 3H), 7.97 (dd, J=4.8, 1.6 Hz, 1H), 7.44 (d, J=7.2 Hz, 2H), 7.39-7.34 (m, 2H), 7.30 (d, J=8.0 Hz, 1H), 7.09 (dd, J=4.8, 1.6 Hz, 1H), 6.94 (br s, 2H), 6.67 (d, J=6.0 Hz, 1H), 6.35 (dd, J=7.6, 4.8 Hz, 1H), 3.85-3.75 (m, 1H), 3.57 (s, 2H), 3.22-3.18 (m, 1H), 2.85-2.76 (m, 2H), 2.19-2.10 (m, 2H), 2.00-1.95 (m, 2H), 1.90-1.76 (m, 2H), 1.75-1.68 (m, 4H), 1.62-1.57 (m, 2H), 1.52-1.43 (m, 2H).

### Example 389: 4-((1-(4-(2-(2-Aminopyridin-3-yl)-5-(dimethylamino)-3H-imidazo[4,5-b]pyridin-3-yl)benzyl)piperidin-4-yl)amino)pyrimidine-2-carbonitrile

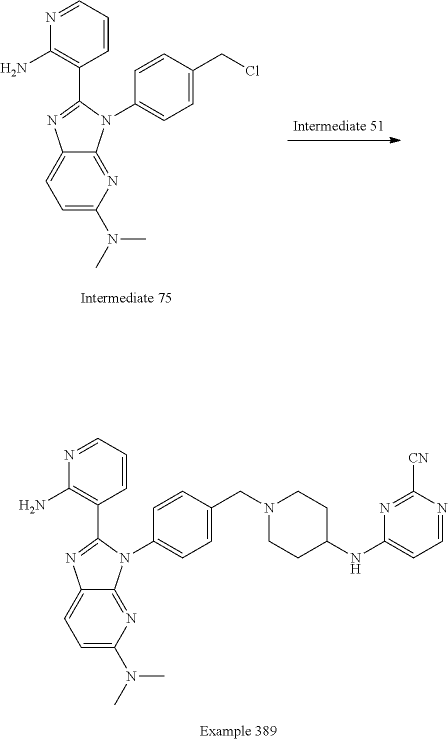 [SMILES: CN(C)c1ccc2nc(-c3cccnc3N)n(-c3ccc(CCl)cc3)c2n1 ; CN(C)c1ccc2nc(-c3cccnc3N)n(-c3ccc(CN4CCC(Nc5ccnc(C#N)n5)CC4)cc3)c2n1]

To a solution of Intermediate 75 (300 mg, 792 μmol) and Intermediate 51 (161 mg TFA salt, 792 μmol) in DMF (5 mL) were added K₂CO₃ (547 mg, 3.96 mmol) and NaI (59.4 mg, 396 μmol). The mixture was stirred at 25° C. for 16 hr. The mixture was filtered, and the filtrate was concentrated to give the crude. After purified by prep-HPLC (column: Waters xbridge 150*25 mm 10 μm; mobile phase: [water (NH₄HCO₃)-ACN]; gradient: 33%-63% B over 14 min), 4-((1-(4-(2-(2-aminopyridin-3-yl)-5-(dimethylamino)-3H-imidazo[4,5-b]pyridin-3-yl)benzyl)piperidin-4-yl)amino)pyrimidine-2-carbonitrile (Example 389, 86.6 mg, yield: 20%) was obtained as a yellow powder. MS: m/z=546.4 [M+H]⁺. ¹H NMR (400 MHz, Methanol-d₄) δ 8.06-7.95 (m, 1H), 7.92 (dd, J=4.8, 1.6 Hz, 1H), 7.86 (d, J=9.2 Hz, 1H), 7.47 (d, J=8.4 Hz, 2H), 7.36 (d, J=8.4 Hz, 2H), 7.21 (dd, J=7.6, 1.6 Hz, 1H), 6.71 (d, J=9.2 Hz, 1H), 6.63-6.56 (m, 1H), 6.44 (dd, J=7.6, 4.8 Hz, 1H), 4.09-3.79 (m, 1H), 3.63 (s, 2H), 3.07 (s, 6H), 2.96-2.93 (m, 2H), 2.30-2.20 (m, 2H), 1.98-1.04 (m, 2H), 1.55-1.64 (m, 2H).

### Example 390: 4-((1-(4-(2-(2-Aminopyridin-3-yl)-3H-imidazo[4,5-b]pyridin-3-yl)benzyl)piperidin-4-yl)(methyl)amino)pyrimidine-2-carbonitrile

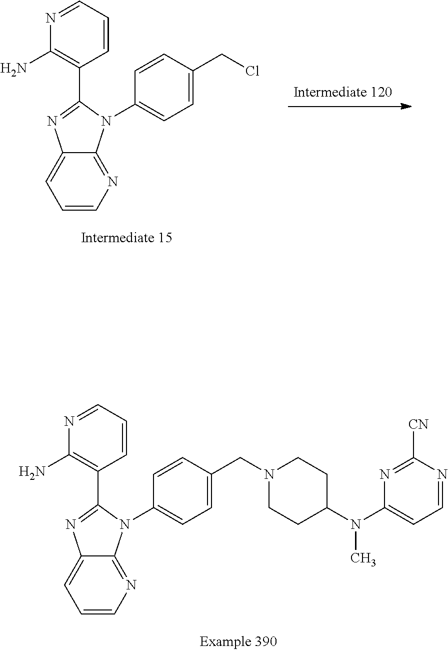 [SMILES: Nc1ncccc1-c1nc2cccnc2n1-c1ccc(CCl)cc1 ; CN(c1ccnc(C#N)n1)C1CCN(Cc2ccc(-n3c(-c4cccnc4N)nc4cccnc43)cc2)CC1]

To a solution of Intermediate 15 (150 mg, 447 μmol) and Intermediate 120 (116 mg, 536 μmol) in DMF (3 mL) were added NaI (6.7 mg, 45 μmol) and K₂CO₃ (185 mg, 1.3 mmol). The mixture was stirred at 50° C. for 1 hr. After purified by silica gel flash chromatography (Eluent of 0˜7% MeOH in CH₂Cl₂), 4-((1-(4-(2-(2-Aminopyridin-3-yl)-3H-imidazo[4,5-b]pyridin-3-yl)benzyl)piperidin-4-yl)(methyl)amino)pyrimidine-2-carbonitrile (Example 390, 176.7 mg, yield: 98%) was obtained as a light-yellow solid. MS: m/z=517.2 [M+H]⁺. ¹H NMR (400 MHz, Dimethylsulfoxide-d₆) δ 8.33 (d, J=4.4 Hz, 1H), 8.26-8.18 (m, 2H), 7.99 (d, J=4.0 Hz, 1H), 7.49-7.44 (m, 2H), 7.42-7.36 (m, 3H), 7.17 (d, J=7.6 Hz, 1H), 7.08-6.84 (m, 3H), 6.38 (dd, J=7.2, 4.8 Hz, 1H), 4.91-4.34 (m, 1H), 3.59 (s, 2H), 2.99-2.84 (m, 5H), 2.23-2.10 (m, 2H), 1.91-1.77 (m, 2H), 1.65-1.53 (m, 2H).

### Example 391: 4-((1-(4-(2-(2-Aminopyridin-3-yl)-5-phenyl-3H-imidazo[4,5-b]pyridin-3-yl)benzyl)piperidin-4-yl)amino)-1,3,5-triazine-2-carbonitrile

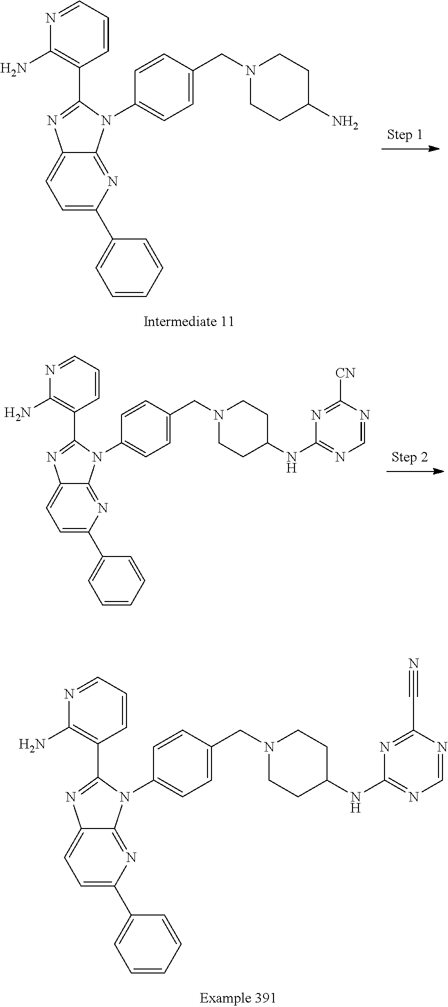 [SMILES: Nc1ncccc1-c1nc2ccc(-c3ccccc3)nc2n1-c1ccc(CN2CCC(N)CC2)cc1 ; N#Cc1ncnc(NC2CCN(Cc3ccc(-n4c(-c5cccnc5N)nc5ccc(-c6ccccc6)nc54)cc3)CC2)n1 ; N#Cc1ncnc(NC2CCN(Cc3ccc(-n4c(-c5cccnc5N)nc5ccc(-c6ccccc6)nc54)cc3)CC2)n1]

### Step 1: N-(1-(4-(2-(2-Aminopyridin-3-yl)-5-phenyl-3H-imidazo[4,5-b]pyridin-3-yl)benzyl)piperidin-4-yl)-4-chloro-1,3,5-triazin-2-amine

To a solution of Intermediate 11 (200 mg, 404 μmol) in THF (4 mL) and H₂O (1 mL) was added K₂CO₃ (148 mg, 1.07 mmol) and stirred at 0° C. for 15 min. Then 2,4-dichloro-1,3,5-triazine (60.6 mg, 404 μmol) in THF (1 mL) was added into the mixture and stirred at 0° C. for 1.5 hr. The reaction was filtered to give the filter liquor and concentrated under reduced pressure. The residue was purified by prep-TLC (MeOH:CH₂Cl₂=1:10) to give N-(1-(4-(2-(2-aminopyridin-3-yl)-5-phenyl-3H-imidazo[4,5-b]pyridin-3-yl)benzyl)piperidin-4-yl)-4-chloro-1,3,5-triazin-2-amine (6.3 mg, yield: 2.4%) as a light-yellow lyophilized powder. MS: m/z=589.3 [M+H]⁺. ¹H NMR (400 MHz, Dimethylsulfoxide-d₆) δ 8.71 (br s, 1H), 8.49-8.35 (m, 1H), 8.27 (d, J=8.4 Hz, 1H), 8.06-7.95 (m, 5H), 7.55-7.35 (m, 6H), 7.16 (d, J=7.4 Hz, 1H), 7.02 (br s, 2H), 6.41-6.33 (m, 1H), 3.81-3.65 (m, 1H), 3.57 (s, 2H), 2.89-2.80 (m, 2H), 2.12-2.02 (m, 2H), 1.86-1.77 (m, 2H), 1.60-1.50 (m, 2H).

### Step 2: To a solution of N-(1-(4-(2-(2-aminopyridin-3-yl)-5-phenyl-3H-imidazo[4,5-b]pyridin-3-yl)benzyl)piperidin-4-yl)-4-chloro-1,3,5-triazin-2-amine

(150 mg, 255 μmol) and DABCO (5.71 mg, 51 μmol) in DMSO (1 mL) was added KCN (49.7 mg, 764 μmol). Then the mixture was stirred at 25° C. for 16 hr. The mixture was quenched with H₂O (15 mL) and extracted with CH₂Cl₂ (15 mL×3). The combined organic layers were dried over anhydrous Na₂SO₄, filtered and concentrated. The residue was purified by prep-HPLC (column: Waters xbridge 150×25 mm 10 μm; mobile phase: [water (NH₄HCO₃)-ACN]; B %: 49%-79%, 10 min) to give 4-((1-(4-(2-(2-aminopyridin-3-yl)-5-phenyl-3H-imidazo[4,5-b]pyridin-3-yl)benzyl)piperidin-4-yl)amino)-1,3,5-triazine-2-carbonitrile (Example 391, 2.9 mg, yield: 1.8%) as a yellow solid. MS: m/z=580.2 [M+H]⁺. ¹H NMR (400 MHz, Acetonitrile-d₃) δ 8.61-8.47 (m, 1H), 8.17 (d, J=8.4 Hz, 1H), 8.06-7.98 (m, 3H), 7.90 (d, J=8.4 Hz, 1H), 7.52-7.39 (m, 7H), 7.16 (dd, J=7.6, 1.6 Hz, 1H), 6.94-6.74 (m, 1H), 6.57 (br s, 2H), 6.42-6.33 (m, 1H), 3.96-3.78 (m, 1H), 3.62 (s, 2H), 2.96-2.84 (m, 2H), 2.25-2.18 (m, 2H), 1.66-1.57 (m, 2H), 1.34-1.21 (m, 2H).

### Example 392: 4-((1-(4-(2-(2-Aminopyridin-3-yl)-3H-imidazo[4,5-b]pyridin-3-yl)benzyl)-4-methylpiperidin-4-yl)amino)pyrimidine-2-carbonitrile

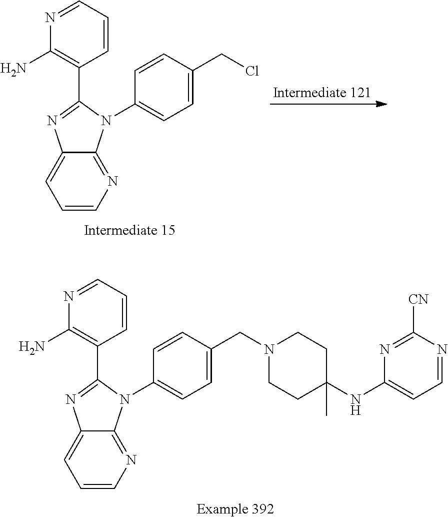 [SMILES: Nc1ncccc1-c1nc2cccnc2n1-c1ccc(CCl)cc1 ; CC1(Nc2ccnc(C#N)n2)CCN(Cc2ccc(-n3c(-c4cccnc4N)nc4cccnc43)cc2)CC1]

To a solution of Intermediate 15 (80 mg, 238 μmol) and Intermediate 121 (62 mg, 286 μmol) in DMF (3 mL) were added KI (4 mg, 24 μmol) and K₂CO₃ (99 mg, 715 μmol). The mixture was stirred at 50° C. for 1 hr. The reaction mixture was poured into H₂O (10 mL), extracted with EtOAc (10 mL×2). The combined organic layers were dried over anhydrous Na₂SO₄, filtered and concentrated under reduced pressure. The crude was purified by silica gel flash chromatography (Eluent of 0˜7% MeOH in CH₂Cl₂) to give 4-((1-(4-(2-(2-aminopyridin-3-yl)-3H-imidazo[4,5-b]pyridin-3-yl)benzyl)-4-methylpiperidin-4-yl)amino)pyrimidine-2-carbonitrile (Example 392, 27.2 mg, yield: 22%) as a light-yellow solid. MS: m/z=517.2 [M+H]⁺. ¹H NMR (400 MHz, Dimethylsulfoxide-d₆) δ 8.33 (dd, J=4.8, 1.3 Hz, 1H), 8.20 (dd, J=8.0, 1.6 Hz, 1H), 8.09 (d, J=6.0 Hz, 1H), 8.00-7.97 (m, 1H), 7.61 (br s, 1H), 7.46-7.42 (m, 2H), 7.41-7.34 (m, 3H), 7.16 (dd, J=7.6, 1.6 Hz, 1H), 7.08-6.93 (m, 2H), 6.81 (d, J=6.4 Hz, 1H), 6.36 (dd, J=8.0, 5.2 Hz, 1H), 3.57-3.48 (m, 2H), 3.30 (s, 2H), 2.33-2.17 (m, 4H), 1.73-1.62 (m, 2H), 1.40 (s, 3H).

### Example 393: 4-((1-(4-(2-(2-Aminopyridin-3-yl)-5-(1-methyl-6-oxo-1,6-dihydropyridin-2-yl)-3H-imidazo[4,5-b]pyridin-3-yl)benzyl)piperidin-4-yl)amino)pyrimidine-2-carbonitrile

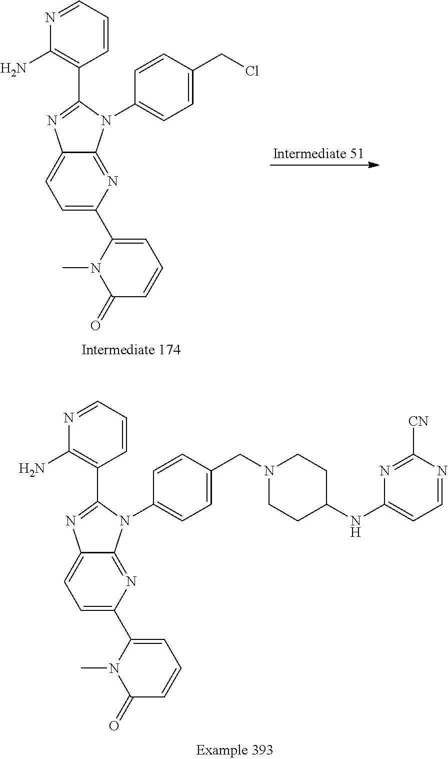 [SMILES: Cn1c(-c2ccc3nc(-c4cccnc4N)n(-c4ccc(CCl)cc4)c3n2)cccc1=O ; Cn1c(-c2ccc3nc(-c4cccnc4N)n(-c4ccc(CN5CCC(Nc6ccnc(C#N)n6)CC5)cc4)c3n2)cccc1=O]

To a solution of Intermediate 174 (120 mg, 271 μmol) and Intermediate 51 (55.1 mg, 271 μmol) in DMF (2 mL) were added NaI (8.12 mg, 54.2 μmol) and K₂CO₃ (112 mg, 813 μmol). The mixture was stirred at 25° C. for 16 hr. The reaction mixture was filtered. After purified by prep-HPLC (column: Phenomenex C18 80*40 mm*3 μm; mobile phase: [water (NH₃H₂O+NH₄HCO₃)-ACN]; gradient: 27%-57% B over 7 min), 4-((1-(4-(2-(2-aminopyridin-3-yl)-5-(1-methyl-6-oxo-1,6-dihydropyridin-2-yl)-3H-imidazo[4,5-b]pyridin-3-yl)benzyl)piperidin-4-yl)amino)pyrimidine-2-carbonitrile (Example 393, 30.2 mg, yield: 18%) was obtained as a yellow lyophilized powder. MS: m/z=610.5 [M+H]⁺. ¹H NMR (400 MHz, Dimethylsulfoxide-d₆) δ 8.36 (d, J=8.0 Hz, J H), 8.12-8.03 (m, 2H), 8.01 (dd, J=4.8, 2.0 Hz, 1H), 7.66 (d, J=8.4 Hz, 1H), 7.49-7.40 (m, 5H), 7.19 (dd, J=7.6, 1.6 Hz, 1H), 7.00 (br s, 2H), 6.66 (d, J=6.0 Hz, 1H), 6.47 (dd, J=9.2, 1.2 Hz, 1H), 6.40 (dd, J=8.0, 4.8 Hz, 1H), 6.33 (dd, J=6.8, 1.2 Hz, 1H), 3.84-3.73 (m, 1H), 3.55 (s, 2H), 3.30 (s, 3H), 2.84-2.76 (m, 2H), 2.18-2.09 (m, 2H), 1.89-1.85 (m, 2H), 1.54-1.42 (m, 2H).

### Example 394: 4-(4-(4-(2-(2-Aminopyridin-3-yl)-5-(1-methyl-6-oxo-1,6-dihydropyridin-2-yl)-3H-imidazo[4,5-b]pyridin-3-yl)benzyl)piperazin-1-yl)pyrimidine-2-carbonitrile

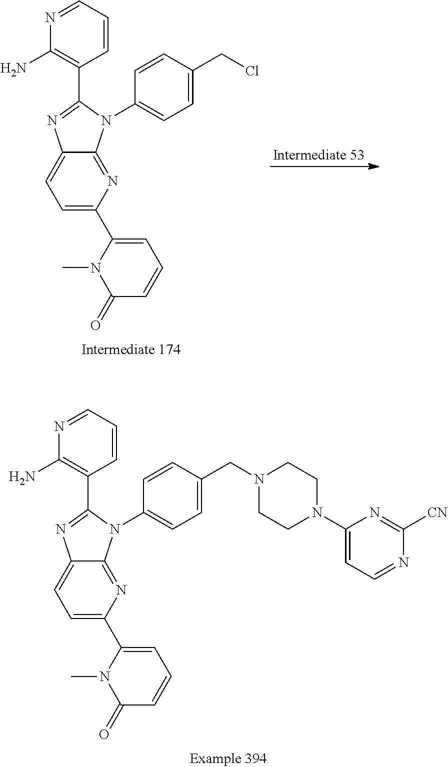 [SMILES: Cn1c(-c2ccc3nc(-c4cccnc4N)n(-c4ccc(CCl)cc4)c3n2)cccc1=O ; Cn1c(-c2ccc3nc(-c4cccnc4N)n(-c4ccc(CN5CCN(c6ccnc(C#N)n6)CC5)cc4)c3n2)cccc1=O]

To a solution of Intermediate 174 (120 mg, 271 μmol) and Intermediate 53 (51.3 mg, 271 μmol) in DMF (2 mL) were added NaI (8.12 mg, 54.2 μmol), K₂CO₃ (112 mg, 813 μmol). The mixture was stirred at 25° C. for 16 hr. The reaction mixture was filtered. After purified by prep-HPLC (column: Phenomenex C18 80*40 mm*3 μm; mobile phase: [water (NH₃H₂O+NH₄HCO₃)-ACN]; gradient: 40%-70% B over 7 min), 4-(4-(4-(2-(2-aminopyridin-3-yl)-5-(1-methyl-6-oxo-1,6-dihydropyridin-2-yl)-3H-imidazo[4,5-b]pyridin-3-yl)benzyl)piperazin-1-yl)pyrimidine-2-carbonitrile (Example 394, 24.7 mg, yield: 14%) was obtained as a yellow lyophilized powder. MS: m/z=596.1 [M+H]⁺. ¹H NMR (400 MHz, Dimethylsulfoxide-d₆) δ 8.37 (d, J=8.4 Hz, 1H), 8.31-8.24 (m, 1H), 8.05-7.97 (m, 1H), 7.66 (d, J=8.0 Hz, 1H), 7.51-7.42 (m, 5H), 7.21-7.17 (m, 1H), 7.11 (d, J=6.0 Hz, 1H), 7.00 (br s, 2H), 6.48 (d, J=9.2 Hz, 1H), 6.40 (dd, J=7.6, 4.8 Hz, 1H), 6.33 (d, J=6.8 Hz, 1H), 3.75-3.64 (m, 4H), 3.62 (s, 2H), 3.31 (s, 3H), 2.48-2.44 (m, 4H).

### Example 395: 4-((1-(4-(2-(2-Aminopyridin-3-yl)-5-(3-methoxyphenyl)-3H-imidazo[4,5-b]pyridin-3-yl)benzyl)piperidin-4-yl)amino)pyrimidine-2-carbonitrile

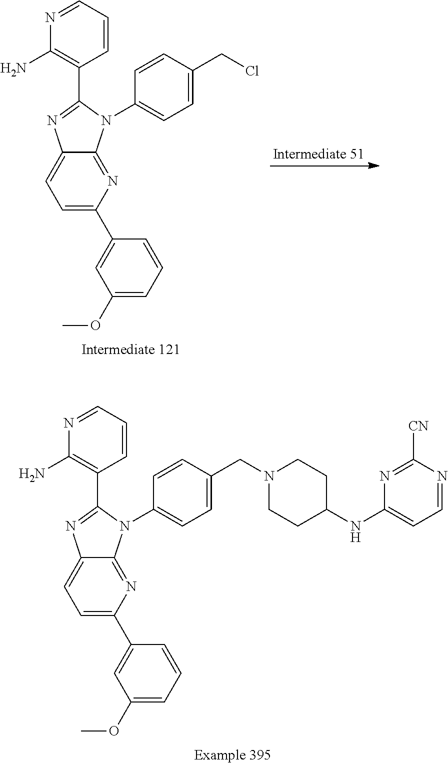 [SMILES: COc1cccc(-c2ccc3nc(-c4cccnc4N)n(-c4ccc(CCl)cc4)c3n2)c1 ; COc1cccc(-c2ccc3nc(-c4cccnc4N)n(-c4ccc(CN5CCC(Nc6ccnc(C#N)n6)CC5)cc4)c3n2)c1]

To a solution of Intermediate 122 (250 mg, 566 μmol) and Intermediate 51 (126 mg, 622 μmol) in DMF (5 mL) were added NaI (17 mg, 113 μmol) and K₂CO₃ (235 mg, 1.7 mmol). The mixture was stirred at 50° C. for 2 hr. The reaction mixture was diluted with H₂O (15 mL) and extracted with EtOAc (20 mL). The organic layers were washed with brine (10 mL), dried over Na₂SO₄, filtered and concentrated under reduced pressure. After purified by prep-HPLC (column: Phenomenex C18 80*40 mm*3 um; mobile phase: [water (NH₄HCO₃)-ACN]; gradient: 54%-84% B over 8 min), 4-((1-(4-(2-(2-aminopyridin-3-yl)-5-(3-methoxyphenyl)-3H-imidazo[4,5-b]pyridin-3-yl)benzyl)piperidin-4-yl)amino)pyrimidine-2-carbonitrile (Example 395, 29 mg, yield: 8%) was obtained as a yellow solid. MS: m/z=609.5 [M+H]⁺. ¹H NMR (400 MHz, Dimethylsulfoxide-d₆) δ 8.26 (d, J=8.4 Hz, 1H), 8.17-8.02 (m, 2H), 8.02-7.98 (m, 2H), 7.63-7.57 (m, 2H), 7.50-7.43 (m, 4H), 7.41-7.35 (m, 1H), 7.18 (dd, J=8.0, 2.0 Hz, 1H), 7.11-7.01 (m, 2H), 6.97 (dd, J=8.0, 2.0 Hz, 1H), 6.68 (d, J=6.0 Hz, 1H), 6.39 (dd, J=8.0, 4.8 Hz, 1H), 3.85-3.80 (m, 1H), 3.79 (s, 3H), 3.59 (s, 2H), 3.30-3.29 (m, 2H), 2.86-2.79 (m, 2H), 2.19-2.11 (m, 2H), 1.93-1.84 (m, 2H).

### Example 396: 4-((1-(4-(2-(2-Aminopyridin-3-yl)-5-(tert-butyl)-3H-imidazo[4,5-b]pyridin-3-yl)benzyl)piperidin-4-yl)amino)pyrimidine-2-carbonitrile

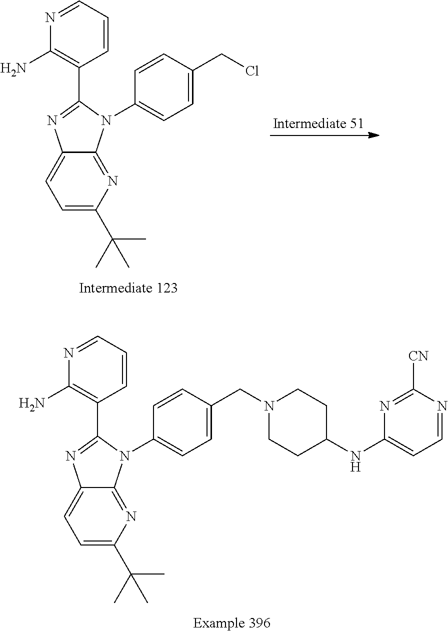 [SMILES: CC(C)(C)c1ccc2nc(-c3cccnc3N)n(-c3ccc(CCl)cc3)c2n1 ; CC(C)(C)c1ccc2nc(-c3cccnc3N)n(-c3ccc(CN4CCC(Nc5ccnc(C#N)n5)CC4)cc3)c2n1]

To a solution of Intermediate 123 (200 mg, 510 mmol) and Intermediate 51 (178 mg TFA salt, 561 μmol) in DMF (4 mL) were added K₂CO₃ (353 mg, 2.55 mmol) and NaI (23 mg, 153 μmol), The mixture was stirred at 50° C. for 2 hr. The reaction mixture was poured into H₂O (15 mL), extracted with EtOAc (15 mL×3). The combined organic layers were dried overanhydrous Na₂SO₄, filtered and concentrated under reduced pressure. After purified by silica gel flash chromatography (Eluent of 0˜18%, MeOH in CH₂Cl₂), 4-((1-(4-(2-(2-aminopyridin-3-yl)-5-(tert-butyl)-3H-imidazo[4,5-b]pyridin-3-yl)benzyl)piperidin-4-yl)amino)pyrimidine-2-carbonitrile (Example 396, 60.4 mg, yield: 21%) was obtained as a light-yellow solid. MS: m/z=559.2 [M+H]⁺. ¹H NMR (400 MHz, Acetonitrile-d₃) δ 8.17-7.87 (m, 3H), 7.49-7.43 (m, 3H), 7.36 (d, J=8.0 Hz, 2H), 7.09 (d, J=7.6 Hz, 1H) 6.63-6.54 (m, 1H), 6.48 (br s, 2H), 6.36 (dd, J=8.0, 5.2 Hz, 1H), 6.33-6.20 (m, 1H), 4.06-3.76 (m, 1H), 3.63 (s, 2H), 2.92-2.84 (m, 2H), 2.31-2.27 (m, 2H), 2.02-1.97 (m, 2H), 1.62-1.52 (m, 2H), 1.34 (s, 9H).

### Example 397: 4-((1-(4-(2-(2-Aminopyridin-3-yl)-5-ethyl-3H-imidazo[4,5-b]pyridin-3-yl)benzyl)piperidin-4-yl)amino)pyrimidine-2-carbonitrile

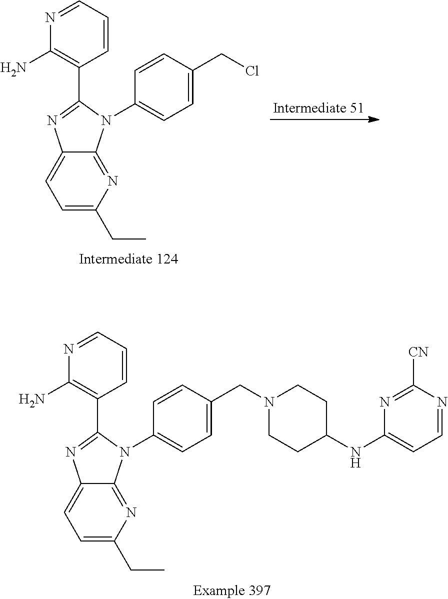 [SMILES: CCc1ccc2nc(-c3cccnc3N)n(-c3ccc(CCl)cc3)c2n1 ; CCc1ccc2nc(-c3cccnc3N)n(-c3ccc(CN4CCC(Nc5ccnc(C#N)n5)CC4)cc3)c2n1]

To a solution of Intermediate 124 (170 mg HCl salt, 424 μmol) and Intermediate 51 (161 mg TFA salt, 509 μmol) in DMF (2 mL) were added NaI (31.8 mg, 212 μmol) and K₂CO₃ (293 mg, 2.12 mmol). The mixture was stirred at 50° C. for 1 hr. The reaction mixture was diluted with water (10 mL) and extracted with EtOAc (2×20 mL). The combined organic layers were washed with brine (30 mL), dried over Na₂SO₄, filtered and concentrated under reduced pressure. After purified by silica gel flash chromatography (Eluent of 0˜5%, MeOH in CH₂Cl₂), 4-((1-(4-(2-(2-aminopyridin-3-yl)-5-ethyl-3H-imidazo[4,5-b]pyridin-3-yl)benzyl)piperidin-4-yl)amino)pyrimidine-2-carbonitrile (Example 397, 131.7 mg, yield: 57%) was obtained as an off white solid. MS: m/z=531.2 [M+H]⁺. ¹H NMR (400 MHz, Dimethylsulfoxide-d₆) δ 8.12-8.01 (m, 3H), 7.97 (dd, J=4.8, 2.0 Hz, 1H), 7.47-7.42 (m, 2H), 7.38-7.34 (m, 2H), 7.28 (d, J=8.4 Hz, 1H), 7.08 (dd, J=7.6, 1.6 Hz, 1H), 6.97 (br s, 2H), 6.67 (d, J=6.0 Hz, JH), 6.34 (dd, J=7.2, 4.4 Hz, 1H), 3.89-7.73 (m, 1H), 3.57 (s, 2H), 2.84-2.76 (m, 4H), 2.18-2.12 (m, 2H), 1.95-1.81 (m, 2H), 1.55-1.44 (m, 2H), 1.21 (t, J=7.6 Hz, 3H).

### Example 398: 2-(2-Aminopyridin-3-yl)-3-(4-((4-((2-cyanopyrimidin-4-yl)amino)piperidin-1-yl)methyl)phenyl)-N-methyl-3H-imidazo[4,5-b]pyridine-5-carboxamide

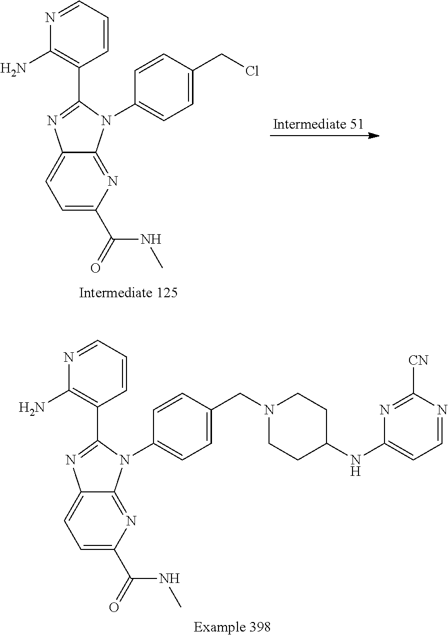 [SMILES: CNC(=O)c1ccc2nc(-c3cccnc3N)n(-c3ccc(CCl)cc3)c2n1 ; CNC(=O)c1ccc2nc(-c3cccnc3N)n(-c3ccc(CN4CCC(Nc5ccnc(C#N)n5)CC4)cc3)c2n1]

To a solution of Intermediate 125 (200 mg, 509 μmol) and Intermediate 51 (103 mg, 326 μmol) in DMF (2 mL) were added K₂CO₃ (352 mg, 2.6 mmol) and NaI (23 mg, 153 μmol). The mixture was stirred at 50° C. for 0.6 hr. The reaction mixture was poured into H₂O (10 mL), extracted with EtOAc (45 mL). The organic phase was dried over anhydrous Na₂SO₄, filtered and concentrated under reduced pressure. After purified by prep-HPLC(column: Waters xbridge 150*25 mm 10 μm; mobile phase: [water (NH₄HCO₃)-ACN]; gradient: 18%-48% B over 14 min), 2-(2-aminopyridin-3-yl)-3-(4-((4-((2-cyanopyrimidin-4-yl)amino)piperidin-1-yl)methyl)phenyl)-N-methyl-3H-imidazo[4,5-b]pyridine-5-carboxamide (Example 398, 84.2 mg, yield: 29%) was obtained as an off-white lyophilized powder. MS: m/z=560.2 [M+H]⁺. ¹H NMR (400 MHz, Dimethylsulfoxide-d₆) δ 8.32 (d, J=8.0 Hz, 1H), 8.14-8.13 (m, 1H), 8.12-8.02 (m, 3H), 8.00 (d, J=4.4 Hz, 1H), 7.51-7.39 (m, 4H), 7.15 (d, J=7.6 Hz, 1H), 6.96 (br s, 2H), 6.67 (d, J=6.0 Hz, 1H), 6.38 (dd, J=7.2, 4.8 Hz, 1H), 3.98-3.69 (m, 1H), 3.57 (s, 2H), 2.89-2.78 (m, 5H), 2.18-2.12 (m, 2H), 1.97-1.81 (m, 2H), 1.52-1.45 (m, 2H).

### Example 399: 4-(4-(4-(2-(2-Aminopyridin-3-yl)-5-(3-cyanopyridin-4-yl)-3H-imidazo[4,5-b]pyridin-3-yl)benzyl)piperazin-1-yl)pyrimidine-2-carbonitrile

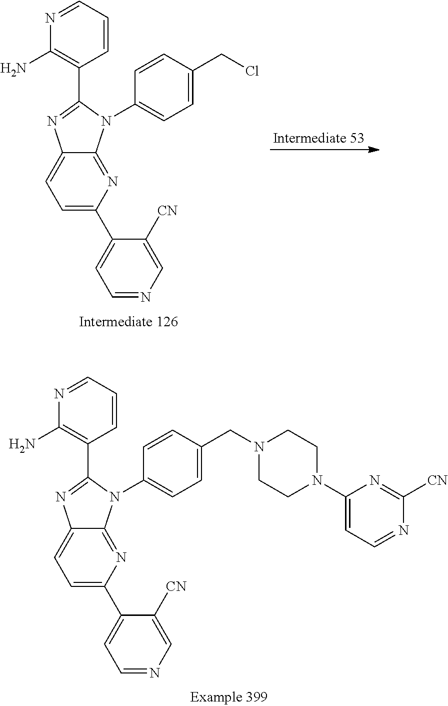 [SMILES: N#Cc1cnccc1-c1ccc2nc(-c3cccnc3N)n(-c3ccc(CCl)cc3)c2n1 ; N#Cc1nccc(N2CCN(Cc3ccc(-n4c(-c5cccnc5N)nc5ccc(-c6ccncc6C#N)nc54)cc3)CC2)n1]

To a solution of Intermediate 126 (200 mg, 456 μmol) and Intermediate 53 (138 mg TFA salt, 456 μmol) in DMF (2 mL) were added NaI (34 mg, 228 μmol) and K₂CO₃ (315 mg, 2.28 μmol). The mixture was stirred at 50° C. for 1 hr. The reaction mixture was filtered. After purified by prep-HPLC (column: [water (NH₄HCO₃)-ACN]; B %: 34%-64%, 14 min), 4-(4-(4-(2-(2-aminopyridin-3-yl)-5-(3-cyanopyridin-4-yl)-3H-imidazo[4,5-b]pyridin-3-yl)benzyl)piperazin-1-yl)pyrimidine-2-carbonitrile (Example 399, 32 mg, yield: 12%) was obtained as a yellow solid. MS: m/z=591.3 [M+H]⁺. ¹H NMR (400 MHz, Dimethylsulfoxide-d₆) δ 9.09 (s, 1H), 8.91 (d, J=5.6 Hz, 1H), 8.45 (d, J=8.4 Hz, 1H), 8.26 (d, J=6.4 Hz, 1H), 8.04-7.99 (m, 3H), 7.51-7.45 (m, 4H), 7.24-7.21 (m, 1H), 7.11 (d, J=6.4 Hz, 1H), 7.01 (br s, 2H), 6.40 (dd, J=7.6, 5.2 Hz, 1H), 3.70-3.60 (m, 6H), 2.48-2.45 (m, 4H).

### Example 400: 4-((1-(4-(2-(2-Aminopyridin-3-yl)-5-(3-cyanopyridin-4-yl)-3H-imidazo[4,5-b]pyridin-3-yl)benzyl)piperidin-4-yl)amino)pyrimidine-2-carbonitrile

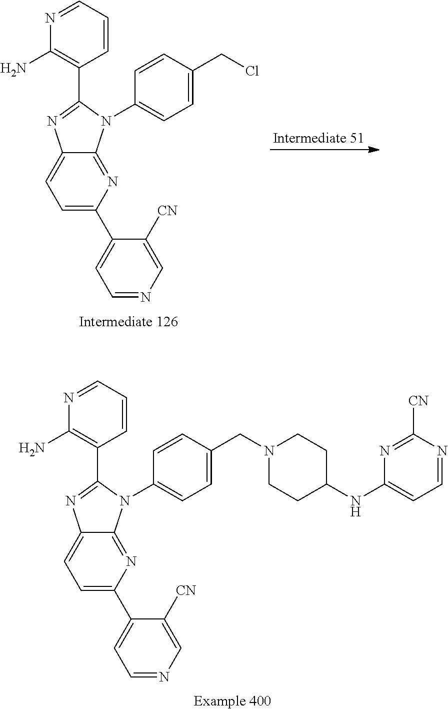 [SMILES: N#Cc1cnccc1-c1ccc2nc(-c3cccnc3N)n(-c3ccc(CCl)cc3)c2n1 ; N#Cc1nccc(NC2CCN(Cc3ccc(-n4c(-c5cccnc5N)nc5ccc(-c6ccncc6C#N)nc54)cc3)CC2)n1]

To a solution of Intermediate 126 (120 mg, 235 μmol) and Intermediate 51 (74.7 mg TFA salt, 235 μmol) in DMF (2 mL) were added NaI (17.6 mg, 117 μmol) and K₂CO₃ (97.7 mg, 707 μmol). The mixture was stirred at 50° C. for 1 hr. The reaction mixture was diluted with water (10 mL) and extracted with EtOAc (2×10 mL). The combined organic layers were washed with brine (10 mL), dried over Na₂SO₄, filtered and concentrated under reduced pressure. The residue was purified by silica gel flash chromatography (Eluent of 0˜6% MeOH in CH₂Cl₂) to give 4-((1-(4-(2-(2-aminopyridin-3-yl)-5-(3-cyanopyridin-4-yl)-3H-imidazo[4,5-b]pyridin-3-yl)benzyl)piperidin-4-yl)amino)pyrimidine-2-carbonitrile (Example 400, 53.7 mg, yield: 36%) as a yellow solid. MS: m/z=605.4 [M+H]⁺. ¹H NMR (400 MHz, Dimethylsulfoxide-d₆) δ 9.08 (s, 1H), 8.91 (d, J=5.2 Hz, 1H), 8.45 (d, J=8.4 Hz, 1H), 8.10-7.96 (m, 5H), 7.52-7.46 (m, 2H), 7.46-7.40 (m, 2H), 7.22 (dd, J=8.0, 1.6 Hz, 1H), 7.01 (br s, 2H), 6.67 (d, J=6.0 Hz, 1H), 6.41 (dd, J=7.6, 4.8 Hz, 1H), 3.91-3.69 (m, 1H), 3.56 (s, 2H), 2.87-2.75 (m, 2H), 2.20-2.05 (m, 2H), 1.93-1.79 (m, 2H), 1.55-1.41 (m, 2H).

### Example 401: 4-((1-(4-(2-(2-Aminopyridin-3-yl)-5-fluoro-3H-imidazo[4,5-b]pyridin-3-yl)benzyl)piperidin-4-yl)amino)pyrimidine-2-carbonitrile

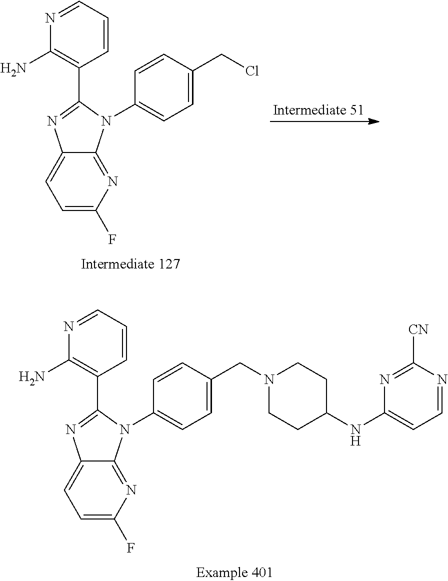 [SMILES: Nc1ncccc1-c1nc2ccc(F)nc2n1-c1ccc(CCl)cc1 ; N#Cc1nccc(NC2CCN(Cc3ccc(-n4c(-c5cccnc5N)nc5ccc(F)nc54)cc3)CC2)n1]

To a solution of Intermediate 127 (200 mg, 565 μmol) and Intermediate 51 (179 mg TFA salt, 565 μmol) in DMF (2 mL) were added NaI (42.3 mg, 282 μmol) and K₂CO₃ (399 mg, 2.83 μmol). The mixture was stirred at 50° C. for 1 hr. The reaction mixture was filtered. After purified by prep-HPLC (column: [water (NH₄HCO₃)-ACN]; B %: 33%-63%, 14 min), 4-((1-(4-(2-(2-aminopyridin-3-yl)-5-fluoro-3H-imidazo[4,5-b]pyridin-3-yl)benzyl)piperidin-4-yl)amino)pyrimidine-2-carbonitrile (Example 401, 51.6 mg, yield: 18%) was obtained as a yellow solid. MS: m/z=521.4 [M+H]⁺. ¹H NMR (400 MHz, Dimethylsulfoxide-d₆) δ 8.41-8.35 (m, 1H), 8.12-8.01 (m, 2H), 7.98 (dd, J=4.8, 2.0 Hz, 1H), 7.47-7.43 (m, 2H), 7.40-7.37 (m, 2H), 7.17-7.14 (m, 2H), 6.91 (br s, 2H), 6.67 (d, J=6.0 Hz, 1H), 6.38 (dd, J=7.6, 4.8 Hz, 1H), 3.84-3.76 (m, 1H), 3.56 (s, 2H), 2.81 (d, J=11.2 Hz, 2H), 2.14 (t, J=10.8 Hz, 2H), 1.91-1.83 (m, 2H), 1.53-1.44 (m, 2H), ¹H NMR (400 MHz, Dimethylsulfoxide-dₕ) 6-74.090.

### Example 402: 4-((1-(4-(2-(2-Aminopyridin-3-yl)-6-fluoro-3H-imidazo[4,5-b]pyridin-3-yl)benzyl)piperidin-4-yl)amino)pyrimidine-2-carbonitrile

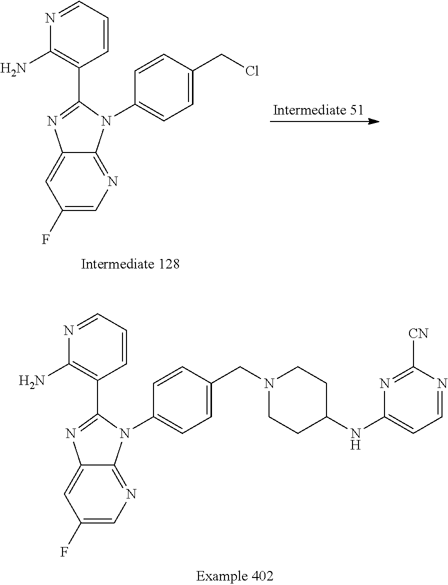 [SMILES: Nc1ncccc1-c1nc2cc(F)cnc2n1-c1ccc(CCl)cc1 ; N#Cc1nccc(NC2CCN(Cc3ccc(-n4c(-c5cccnc5N)nc5cc(F)cnc54)cc3)CC2)n1]

To a solution of Intermediate 128 (284 mg, 803 μmol) and Intermediate 51 (180 mg, 883 μmol) in DMF (2 mL) were added K₂CO₃ (333 mg, 2.4 mmol) and NaI (24 mg, 161 μmol). The mixture was stirred at 50° C. for 0.5 hr. The reaction mixture was diluted with H₂O (20 mL) and extracted with EtOAc (30 mL). The organic layers were washed with brine (30 mL), dried over Na₂SO₄, filtered and concentrated under reduced pressure. After purified by silica gel flash chromatography (Eluent of 0˜5% MeOH in CH₂Cl₂), 4-((1-(4-(2-(2-aminopyridin-3-yl)-6-fluoro-3H-imidazo[4,5-b]pyridin-3-yl)benzyl)piperidin-4-yl)amino)pyrimidine-2-carbonitrile (Example 402, 115.8 mg, yield: 26%) was obtained as a pink solid. MS: m/z=521.1 [M+H]⁺. ¹H NMR (400 MHz, Acetonitrile-d₃) δ 8.26 (s, 1H), 8.21-7.95 (m, 2H), 7.90 (dd, J=8.8, 2.8 Hz, 1H), 7.51 (d, J=8.8 Hz, 2H), 7.35 (d, J=8.8 Hz, 2H), 7.16 (d, J=8.8 Hz, 1H), 6.75-6.49 (m, 3H), 6.37 (dd, J=8.8, 2.8 Hz, 1H), 6.27 (hr s, 1H), 4.02-3.83 (m, 1H), 3.63 (s, 2H), 2.88 (d, J=11.2 Hz, 2H), 2.26-2.21 (m, 2H), 2.17-2.13 (m, 2H), 1.60-1.49 (m, 2H). ¹⁹F NMR (376 MHz, Acetonitrile-d₃) 6-136.38.

### Example 403: 4-((1-(4-(2-(2-Aminopyridin-3-yl)-5-(2-oxooxazolidin-3-yl)-3H-imidazo[4,5-b]pyridin-3-yl)benzyl)piperidin-4-yl)amino)pyrimidine-2-carbonitrile

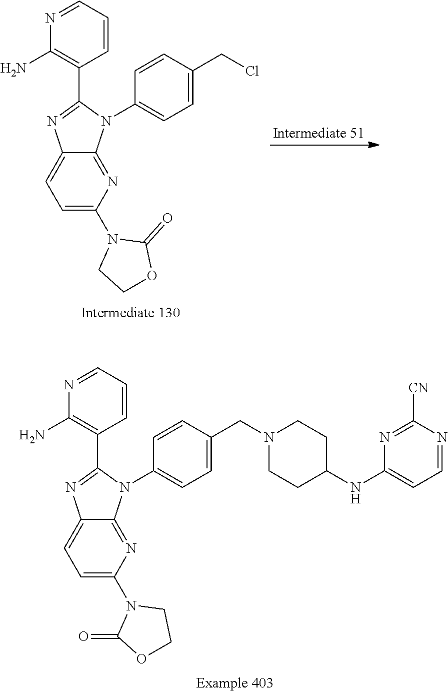 [SMILES: Nc1ncccc1-c1nc2ccc(N3CCOC3=O)nc2n1-c1ccc(CCl)cc1 ; N#Cc1nccc(NC2CCN(Cc3ccc(-n4c(-c5cccnc5N)nc5ccc(N6CCOC6=O)nc54)cc3)CC2)n1]

To a solution of Intermediate 130 (90 mg HCl salt, 196 μmol) and Intermediate 51 (63 mg TFA salt, 196 μmol) in DMF (3 mL) w added K₂CO₃ (136 mg, 984 μmol) and NaI (30 mg, 196 μmol). The mixture was stirred at 25° C. for 1 hr. The reaction mixture was diluted with H₂O (100 mL) and extracted with EtOAc (50 mL×2). The combined organic layers were washed with brine (100 mL), dried over Na₂SO₄, filtered and concentrated under reduced pressure. The residue was purified by silica gel flash chromatography (Eluent of 0˜7% MeOH in CH₂Cl₂) to give 4-((1-(4-(2-(2-aminopyridin-3-yl)-5-(2-oxooxazolidin-3-yl)-3H-imidazo[4,5-b]pyridin-3-yl)benzyl)piperidin-4-yl)amino)pyrimidine-2-carbonitrile (Example 403, 50.1 mg, yield: 41%) was obtained as a yellow solid. MS: m/z=588.1 [M+H]⁺. ¹H NMR (400 MHz, Dimethylsulfoxide-d₆) δ 8.25 (d, J=8.8 Hz, 1H), 8.15-8.02 (m, 3H), 7.98-7.97 (m, 1H), 7.46-7.42 (m, 2H), 7.40-7.35 (m, 2H), 7.10-7.08 (m, 1H), 6.97 (br s, 2H), 6.67 (d, J=6.0 Hz, 1H), 6.36 (dd, J=7.6, 4.8 Hz, 1H), 4.41 (t, J=8.0 Hz, 2H), 4.07 (t, J=8.0 Hz, 2H), 3.88-3.72 (m, 1H), 3.57 (s, 2H), 2.82-2.79 (m, 2H), 2.18-2.10 (m, 2H), 1.92-1.83 (m, 2H), 1.54-1.44 (m, 2H).

### Example 404: 4-((1-(4-(2-(2-Aminopyridin-3-yl)-5-(6-fluoropyridin-3-yl)-3H-imidazo[4,5-b]pyridin-3-yl)benzyl)piperidin-4-yl)amino)pyrimidine-2-carbonitrile

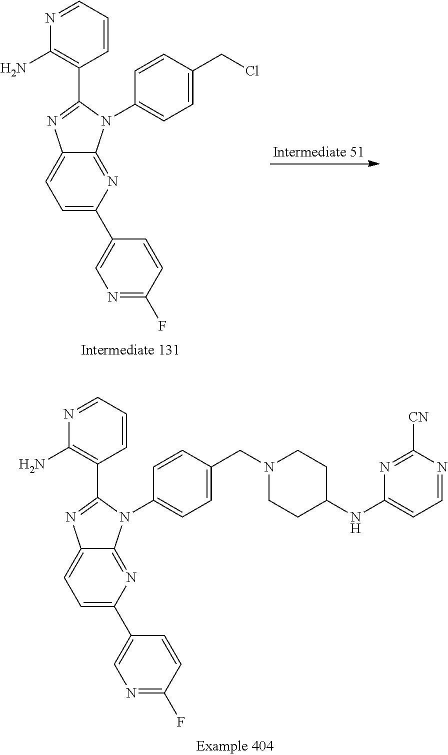 [SMILES: Nc1ncccc1-c1nc2ccc(-c3ccc(F)nc3)nc2n1-c1ccc(CCl)cc1 ; N#Cc1nccc(NC2CCN(Cc3ccc(-n4c(-c5cccnc5N)nc5ccc(-c6ccc(F)nc6)nc54)cc3)CC2)n1]

To a solution of Intermediate 131 (200 mg, 464 μmol) and Intermediate 51 (147 mg, 464 μmol) in DMF (5 mL) were added NaI (34.8 mg, 232 μmol) and K₂CO₃ (321 mg, 2.32 mmol). The mixture was stirred at 50° C. for 1 hr. The reaction mixture was filtered. After purified by prep-HPLC (column: Waters xbridge 150*25 mm 10 μm; mobile phase: [water (NH₄HCO₃)-ACN]; gradient: 38%-68% B over 14 min), 4-((1-(4-(2-(2-aminopyridin-3-yl)-5-(6-fluoropyridin-3-yl)-3H-imidazo[4,5-b]pyridin-3-yl)benzyl)piperidin-4-yl)amino)pyrimidine-2-carbonitrile (Example 404, 56 mg, yield: 20%) was obtained as a yellow solid. MS: m/z=598.4 [M+H]⁺. ¹H NMR (400 MHz, Dimethylsulfoxide-d₆) δ 8.89 (d, J=2.4 Hz, 1H), 8.61-8.53 (m, 1H), 8.33 (d, J=8.4 Hz, 1H), 8.14-8.03 (m, 3H), 8.01 (dd, J=4.8, 1.6 Hz, 1H), 7.50-7.43 (m, 4H), 7.30 (dd, J=8.8, 2.8 Hz, 1H), 7.20-7.14 (m, 1H), 7.04 (br s, 2H), 6.67 (d, J=6.0 Hz, 1H), 6.39 (dd, J=7.6, 5.2 Hz, 1H), 3.85-3.77 (m, 1H), 3.59 (s, 2H), 2.83 (d, J=12.0 Hz, 2H), 2.20-2.11 (m, 2H), 1.93-1.83 (m, 2H), 1.54-1.43 (m, 2H). ¹⁹F NMR (400 MHz, Dimethylsulfoxide-d₆) δ −69.579.

### Example 405: 4-((1-(4-(2-(2-Aminopyridin-3-yl)-5-chloro-3H-imidazo[4,5-b]pyridin-3-yl)benzyl)piperidin-4-yl)amino)pyrimidine-2-carbonitrile

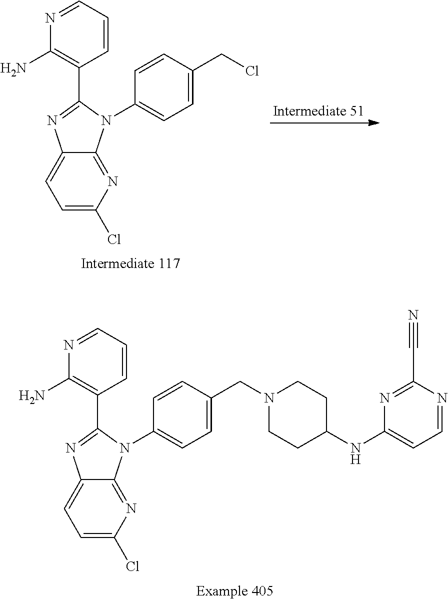 [SMILES: Nc1ncccc1-c1nc2ccc(Cl)nc2n1-c1ccc(CCl)cc1 ; N#Cc1nccc(NC2CCN(Cc3ccc(-n4c(-c5cccnc5N)nc5ccc(Cl)nc54)cc3)CC2)n1]

To a solution of Intermediate 51 (165 mg, 810 μmol) and Intermediate 117 (300 mg, 810 mol) in DMF (3 mL) was added DIEA (314 mg, 2.43 mmol). The mixture was degassed and purged with N₂ three times, and stirred at 25° C. for 16 hr under N₂. The reaction mixture was quenched with H₂O (10 mL) at 25° C. and extracted with CH₂Cl₂ (5 mL×3). The combined organic layers were washed with brine (10 mL×2), dried over Na₂SO₄, filtered and concentrated under reduced pressure. The residue was purified by silica gel flash chromatography (Eluent of 1˜8% MeOH in CH₂Cl₂) to give 4-((1-(4-(2-(2-aminopyridin-3-yl)-5-chloro-3H-imidazo[4,5-b]pyridin-3-yl)benzyl)piperidin-4-yl)amino)pyrimidine-2-carbonitrile (Example 405, 226 mg, yield: 52%) as a light yellow solid. MS: m/z=537.2 [M+H]⁺. ¹H NMR (400 MHz, Dimethylsulfoxide-d₆) δ 8.27 (d, J=8.0 Hz, 1H), 8.14-8.02 (m, 2H), 7.99 (dd, J=4.8, 1.6 Hz, 1H), 7.52-7.36 (m, 5H), 7.16 (dd, J=7.6, 1.6 Hz, 1H), 6.94 (br s, 2H), 6.67 (d, J=6.0 Hz, 1H), 6.38 (dd, J=7.6, 4.8 Hz, 1H), 3.88-3.74 (m, 1H), 3.57 (s, 2H), 2.86-2.75 (m, 2H), 2.20-2.07 (m, 2H), 1.94-1.79 (m, 2H), 1.57-1.40 (m, 2H).

### Example 406: 2-(2-Aminopyridin-3-yl)-3-(4-((4-((2-cyanopyrimidin-4-yl)amino)piperidin-1-yl)methyl)phenyl)-3H-imidazo[4,5-b]pyridine-5-carbonitrile

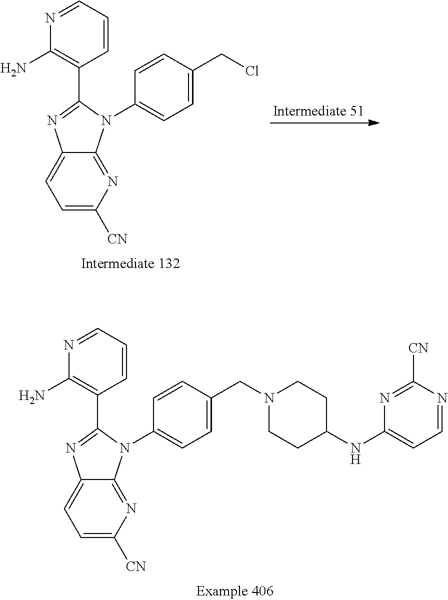 [SMILES: N#Cc1ccc2nc(-c3cccnc3N)n(-c3ccc(CCl)cc3)c2n1 ; N#Cc1ccc2nc(-c3cccnc3N)n(-c3ccc(CN4CCC(Nc5ccnc(C#N)n5)CC4)cc3)c2n1]

To a solution of Intermediate 132 (235 mg, 651 μmol) and Intermediate 51 (207 mg, 651 μmol, TFA) in DMF (5 mL) were added K₂CO₃ (450 mg, 3.26 mmol) and NaI (9.76 mg, 65.1 mol). The mixture was stirred at 60° C. for 1 hr. After purified by prep-HPLC (column: Waters xbridge 150*25 mm*10 μm; mobile phase: [water (NH₄HCO₃)-ACN]; gradient: 25%-55% B over 14 min), 2-(2-aminopyridin-3-yl)-3-(4-((4-((2-cyanopyrimidin-4-yl)amino)piperidin-1-yl)methyl)phenyl)-3H-imidazo[4,5-b]pyridine-5-carbonitrile (Example 406, 36.2 mg, yield: 11%) was obtained as a yellow solid. MS: m/z=528.3 [M+H]⁺. ¹H NMR (400 MHz, Dimethylsulfoxide-d₆) δ 8.42 (d, J=8.0 Hz, 1H), 8.13-7.97 (m, 4H), 7.50-7.41 (m, 4H), 7.22 (d, J=8.0 Hz, 1H), 6.99 (s, 2H), 6.67 (d, J=6.0 Hz, 1H), 6.43-6.33 (m, 1H), 3.92-3.69 (m, 1H), 3.57 (s, 2H), 2.87-2.78 (m, 2H), 2.21-2.12 (m, 2H), 1.94-1.84 (m, 2H), 1.55-1.45 (m, 2H).

### Example 407: 4-(4-(4-(2-(2-Aminopyridin-3-yl)-5-(3-cyanopyridin-2-yl)-3H-imidazo[4,5-b]pyridin-3-yl)benzyl)piperazin-1-yl)pyrimidine-2-carbonitrile

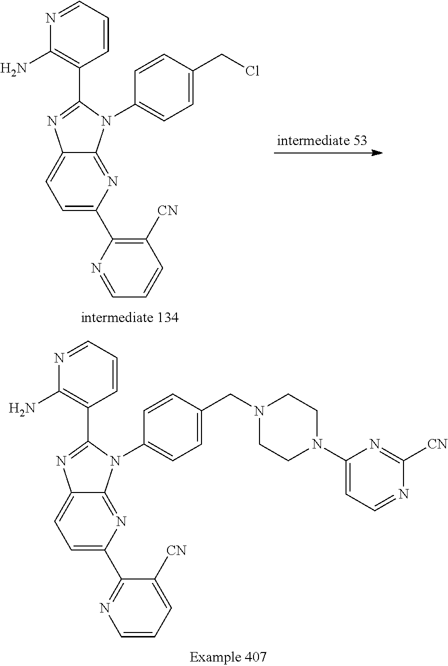 [SMILES: N#Cc1cccnc1-c1ccc2nc(-c3cccnc3N)n(-c3ccc(CCl)cc3)c2n1 ; N#Cc1nccc(N2CCN(Cc3ccc(-n4c(-c5cccnc5N)nc5ccc(-c6ncccc6C#N)nc54)cc3)CC2)n1]

To a solution of Intermediate 134 (100 mg, 211 μmol) and Intermediate 53 (63.9 mg TFA salt, 211 μmol) in DMF (2 mL) were added K₂CO₃ (146 mg, 1.05 mmol) and NaI (9.48 mg, 63.3 μmol). The mixture was stirred at 50° C. for 0.5 hr. The mixture was filtered and concentrated to give the crude. After purified by prep-HPLC (column: Phenomenex C18 80*40 mm*3 μm; mobile phase: [water (NH₄HCO₃)-ACN]; gradient: 49%-79% B over 8 min), 4-(4-(4-(2-(2-aminopyridin-3-yl)-5-(3-cyanopyridin-2-yl)-3H-imidazo[4,5-b]pyridin-3-yl)benzyl)piperazin-1-yl)pyrimidine-2-carbonitrile (Example 407, 31.6 mg delivered, yield: 25%) was obtained as light-yellow solid. MS: m/z=591.1 [M+H]⁺. ¹H NMR (400 MHz, Dimethylsulfoxide-d₆) δ 8.93 (dd, J=4.8, 1.6 Hz, 1H), 8.46-8.35 (m, 2H), 8.29-8.20 (m, 2H), 8.02 (dd, J=4.8, 2.0 Hz, 1H), 7.64 (dd, J=8.0, 4.8 Hz, 1H), 7.54-7.49 (m, 2H), 7.48-7.39 (m, 2H), 7.24 (dd, J=8.0, 2.0 Hz, 1H), 7.11 (d, J=6.4 Hz, 1H), 7.01 (br s, 2H), 6.41 (dd, J=7.6, 4.8 Hz, 1H), 3.71-3.66 (m, 2H), 3.61 (s, 2H), 3.31-3.29 (m, 2H), 2.53-2.51 (m, 2H), 2.48-2.44 (m, 2H).

### Example 408: 4-((1-(4-(2-(2-Aminopyridin-3-yl)-5-(3-cyanopyridin-2-yl)-3H-imidazo[4,5-b]pyridin-3-yl)benzyl)piperidin-4-yl)amino)pyrimidine-2-carbonitrile

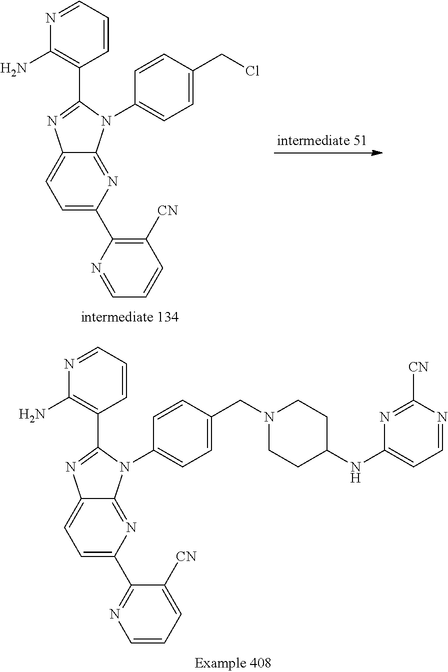 [SMILES: N#Cc1cccnc1-c1ccc2nc(-c3cccnc3N)n(-c3ccc(CCl)cc3)c2n1 ; N#Cc1nccc(NC2CCN(Cc3ccc(-n4c(-c5cccnc5N)nc5ccc(-c6ncccc6C#N)nc54)cc3)CC2)n1]

To a solution of Intermediate 134 (140 mg, 320 μmol) and Intermediate 51 (65.0 mg TFA salt, 320 μmol) in DMF (2 mL) were added K₂CO₃ (221 mg, 1.60 mmol) and NaI (14.4 mg, 95.9 μmol). The mixture was stirred at 50° C. for 0.5 hr. The mixture was filtered, and the filtrate was concentrated to give the crude product. After purified by prep-HPLC (column: Waters xbridge 150*25 mm*10 μm; mobile phase: [water (NH₄HCO₃)-ACN]; gradient: 28%-58% B over 14 min), 4-((1-(4-(2-(2-aminopyridin-3-yl)-5-(3-cyanopyridin-2-yl)-3H-imidazo[4,5-b]pyridin-3-yl)benzyl)piperidin-4-yl)amino)pyrimidine-2-carbonitrile (Example 408, 24.1 mg, yield: 12%) was obtained as light-yellow solid. MS: m/z=605.4 [M+H]⁺. ¹H NMR (400 MHz, Dimethylsulfoxide-d₆) δ 8.93 (dd, J=4.8, 1.6 Hz, 1H), 8.42-8.36 (m, 2H), 8.24 (d, J=8.4 Hz, 1H), 8.10-8.00 (m, 3H), 7.63 (dd, J=7.6, 4.4 Hz, 1H), 7.50 (d, J=8.4 Hz, 2H), 7.42 (d, J=8.4 Hz, 2H), 7.23 (dd, J=7.6, 1.6 Hz, 1H), 7.02 (br s, 2H), 6.72-6.60 (m, 1H), 6.41 (dd, J=7.6, 5.2 Hz, 1H), 3.95-3.68 (m, 1H), 3.56 (s, 2H), 2.86-2.79 (m, 2H), 2.17-2.09 (m, 2H), 1.92-1.83 (m, 2H), 1.52-1.41 (m, 2H).

### Example 409: 4-((1-(4-(2-(2-Aminopyridin-3-yl)-5-(2-cyanopyridin-3-yl)-3H-imidazo[4,5-b]pyridin-3-yl)benzyl)piperidin-4-yl)amino)pyrimidine-2-carbonitrile

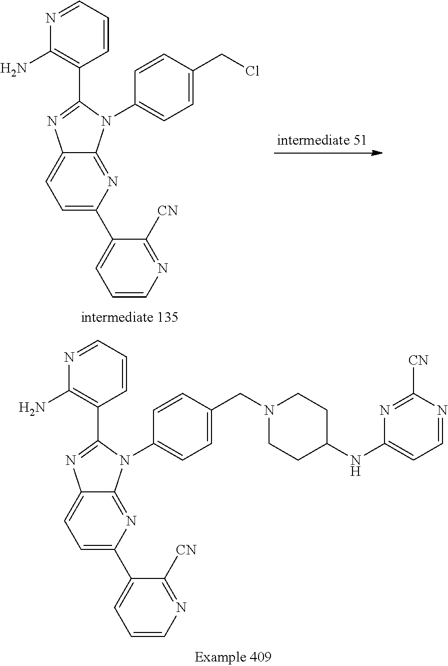

To a solution of Intermediate 135 (100 mg, 228 μmol) and Intermediate 51 (86.9 mg, 274 μmol) in DMF (2 mL) were added NaI (6.85 mg, 45.7 μmol) and K₂CO₃ (189 mg, 1.37 mmol). The mixture was stirred at 50° C. for 2 hr. The reaction mixture was diluted with H₂O (100 mL) and extracted with CH₂Cl₂ (50 mL×2). The combined organic layers were washed with brine (100 mL×2), dried over Na₂SO₄, filtered and concentrated under reduced pressure. After purified by silica gel flash chromatography (Eluent of 0˜10% MeOH in CH₂Cl₂), 4-((1-(4-(2-(2-aminopyridin-3-yl)-5-(2-cyanopyridin-3-yl)-3H-imidazo[4,5-b]pyridin-3-yl)benzyl)piperidin-4-yl)amino)pyrimidine-2-carbonitrile (Example 409, 35.7 mg, yield: 25%) was obtained as a yellow solid. MS: m/z=605.4 [M+H]⁺. ¹H NMR (400 MHz, Dimethylsulfoxide-d₆) δ 8.78 (d, J=4.4 Hz, 1H), 8.43 (d, J=8.0 Hz, 1H), 8.36 (d, J=8.0 Hz, 1H), 8.11-7.99 (m, 3H), 7.94 (d, J=8.0 Hz, 1H), 7.84 (dd, J=8.0, 4.4 Hz, 1H), 7.54-7.38 (m, 4H), 7.21 (d, J=7.2 Hz, 1H), 7.01 (br s, 2H), 6.66 (d, J=5.6 Hz, 1H), 6.41 (dd, J=7.2, 4.8 Hz, 1H), 3.88-3.71 (m, 1H), 3.56 (s, 2H), 2.86-2.73 (m, 2H), 2.22-2.05 (m, 2H), 1.93-1.76 (m, 2H), 1.55-1.40 (m, 2H).

### Example 410: 4-((1-(4-(2-(2-Aminopyridin-3-yl)-5-(trifluoromethyl)-3H-imidazo[4,5-b]pyridin-3-yl)benzyl)piperidin-4-yl)amino)pyrimidine-2-carbonitrile

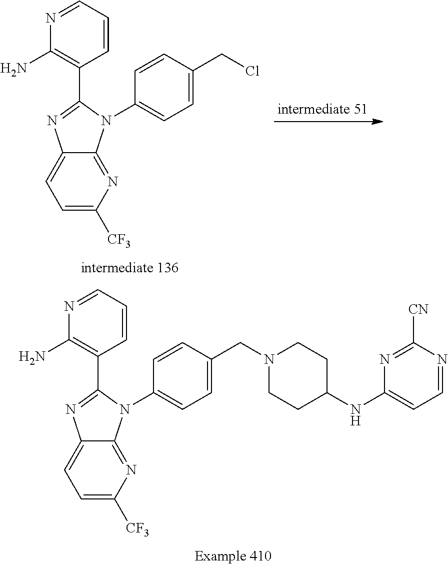 [SMILES: Nc1ncccc1-c1nc2ccc(C(F)(F)F)nc2n1-c1ccc(CCl)cc1 ; N#Cc1nccc(NC2CCN(Cc3ccc(-n4c(-c5cccnc5N)nc5ccc(C(F)(F)F)nc54)cc3)CC2)n1]

To a solution of Intermediate 136 (450 mg, 792 μmol) and Intermediate 51 (335 mg, 1.06 mmol, TFA) in DMF (5 mL) was added K₂CO₃ (424 mg, 3.07 mmol) and NaI (15.3 mg, 102 μmol). The mixture was stirred at 25° C. for 16 hr. The mixture was filtered, and the filtrate was concentrated to give the crude product. After purified by prep-HPLC (column: Phenomenex C18 75*30 mm*3 μm; mobile phase: [water(NH₃H₂O+NH₄HCO₃)-ACN]; gradient: 47%-77% B over 7 min), 4-((1-(4-(2-(2-aminopyridin-3-yl)-5-(trifluoromethyl)-3H-imidazo[4,5-b]pyridin-3-yl)benzyl)piperidin-4-yl)amino)pyrimidine-2-carbonitrile (Example 410, 104.3 mg, yield: 18%) was obtained as a yellow powder. MS: m/z=571.3 [M+H]⁺. ¹H NMR (400 MHz, Dimethylsulfoxide-d₆) δ 8.44 (d, J=8.0 Hz, 1H), 8.24-7.97 (m, 3H), 7.87 (d, J=8.0 Hz, 1H), 7.51-7.40 (m, 4H), 7.21 (dd, J=7.6, 1.6 Hz, 1H), 6.91 (s, 2H), 6.67 (d, J=6.0 Hz, 1H), 6.40 (dd, J=7.6, 4.8 Hz, 1H), 3.96-3.65 (m, J=5.0 Hz, 1H), 3.57 (s, 2H), 2.88-2.76 (m, 2H), 2.23-2.09 (m, 2H), 1.89 (m, 2H), 1.57-1.42 (m, 2H). ¹⁹F NMR (400 MHz, Dimethylsulfoxide-d₆) δ −64.17.

### Example 411: 2-(2-Aminopyridin-3-yl)-3-(4-((4-((2-cyanopyrimidin-4-yl)amino)piperidin-1-yl)methyl)phenyl)-3H-imidazo[4,5-b]pyridine-6-carbonitrile

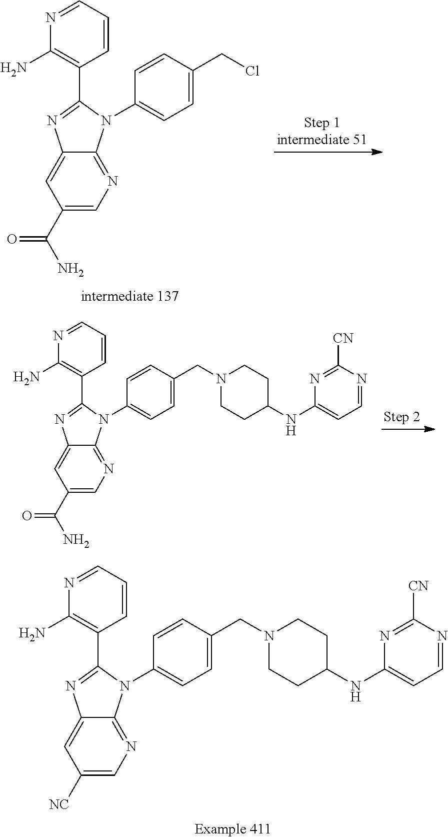

### Step 1: 2-(2-Aminopyridin-3-yl)-3-(4-((4-((2-cyanopyrimidin-4-yl)amino)piperidin-1-yl)methyl)phenyl)-3H-imidazo[4,5-b]pyridine-6-carboxamide

To a solution of Intermediate 137 (270 mg, 713 μmol) and Intermediate 51 (145 mg, 713 μmol) in DMF (5 mL) were added K₂CO₃ (493 mg, 3.56 mmol) and NaI (10.7 mg, 71.3 mol). The mixture was stirred at 60° C. for 1 hr. The reaction mixture was diluted with H₂O (50 mL) and extracted with CH₂Cl₂ (50 mL×2). The combined organic layers were washed with brine (50 mL), dried over Na₂SO₄, filtered and concentrated under reduced pressure to give 2-(2-aminopyridin-3-yl)-3-(4-((4-((2-cyanopyrimidin-4-yl)amino)piperidin-1-yl)methyl)phenyl)-3H-imidazo[4,5-h]pyridine-6-carboxamide (230 mg, yield: 38%) as a yellow solid. MS: m/z=546.3 [M+H]⁺. ¹H NMR (400 MHz, Dimethylsulfoxide-d₆) δ 8.85 (d, J=2.0 Hz, 1H), 8.65 (d, J=1.6 Hz, 1H), 8.22-8.17 (m, 1H), 8.09-8.07 (m, 1H), 8.01-7.99 (m, 1H), 7.61-7.50 (m, 2H), 7.48-7.39 (m, 4H), 7.21-7.17 (m, 1H), 6.95 (s, 2H), 6.70-6.66 (m, 1H), 6.39 (dd, J=7.6, 4.8 Hz, 1H), 3.86-3.77 (m, 1H), 3.56 (s, 2H), 2.85-2.77 (m, 2H), 2.18-2.13 (m, 2H), 1.90-1.85 (m, 2H), 1.50-1.43 (m, 2H).

### Step 2: 2-(2-Aminopyridin-3-yl)-3-(4-((4-((2-cyanopyrimidin-4-yl)amino)piperidin-1-yl)methyl)phenyl)-3H-imidazo[4,5-b]pyridine-6-carbonitrile

To a solution of 2-(2-aminopyridin-3-yl)-3-(4-((4-((2-cyanopyrimidin-4-yl)amino)piperidin-1-yl)methyl)phenyl)-3H-imidazo[4,5-h]pyridine-6-carboxamide (200 mg, 367 μmol) in CH₂Cl₂ (2 mL) was added Burgess reagent (174 mg, 733 μmol). The mixture was stirred at 25° C. for 1 hr. The reaction mixture was quenched with NaHCO₃ (10 mL) at 0° C., and then diluted with H₂O (20 mL) and extracted with CH₂Cl₂ (20 mL×2). The combined organic layers were washed with brine (40 mL), dried over Na₂SO₄, filtered and concentrated under reduced pressure. After purified by prep-HPLC (column: Phenomenex Cj_{R} 75*30 mm*3 μm; mobile phase: [water (NH₃H₂O+NH₄HCO₃)-ACN]; gradient: 33%-63% B over 7 min), 2-(2-aminopyridin-3-yl)-3-(4-((4-((2-cyanopyrimidin-4-yl)amino)piperidin-1-yl)methyl)phenyl)-3H-imidazo[4,5-b]pyridine-6-carbonitrile (Example 411, 6.1 mg, yield: 3.1%) was obtained as a yellow solid. MS: m/z=528.4 [M+H]⁺. ¹H NMR (400 MHz, Acetonitrile-d₃) δ 8.64 (d, J=2.0 Hz, 1H), 8.47 (d, J=2.0 Hz, 1H), 8.06-8.04 (m, 1H), 7.52 (d, J=8.4 Hz, 2H), 7.36 (d, J=8.0 Hz, 2H), 7.19-7.15 (m, 1H), 6.59-6.57 (m, 2H), 6.37 (dd, J=8.0, 4.8 Hz, 1H), 6.29-6.22 (m, 1H), 4.01-3.76 (m, 1H), 3.61 (s, 2H), 2.93-2.78 (m, 2H), 2.26-2.19 (m, 2H), 2.00-1.96 (m, 2H), 1.60-1.46 (m, 2H).

### Example 412: 4-((1-(4-(2-(2-Aminopyridin-3-yl)-5,6-dimethyl-3H-imidazo[4,5-b]pyridin-3-yl)benzyl)piperidin-4-yl)amino)pyrimidine-2-carbonitrile

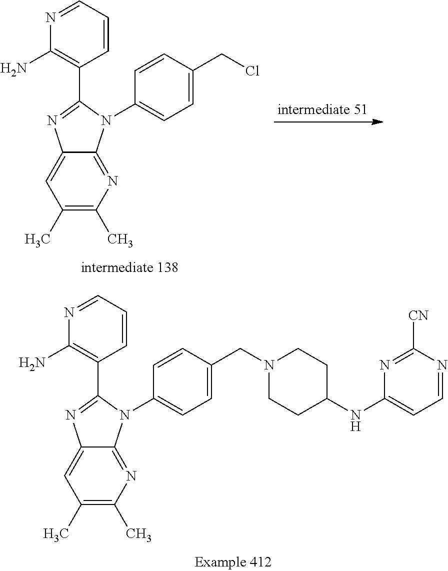 [SMILES: Cc1cc2nc(-c3cccnc3N)n(-c3ccc(CCl)cc3)c2nc1C ; Cc1cc2nc(-c3cccnc3N)n(-c3ccc(CN4CCC(Nc5ccnc(C#N)n5)CC4)cc3)c2nc1C]

To a solution of Intermediate 138 (200 mg, 549 μmol) and Intermediate 51 (174 mg, 549 μmol) in DMF (5 mL) were added NaI (41.2 mg, 275 μmol) and K₂CO₃ (379 mg, 2.75 mmol). The mixture was stirred at 50° C. for 1 hr. The reaction mixture was filtered and purified by prep-HPLC (column: Waters xbridge 150*25 mm 10 μm; mobile phase: [water (NH₄HCO₃)-ACN]; gradient: 37%-67% B over 14 min) to give 4-((1-(4-(2-(2-aminopyridin-3-yl)-5,6-dimethyl-3H-imidazo[4,5-b]pyridin-3-yl)benzyl)piperidin-4-yl)amino)pyrimidine-2-carbonitrile (Example 412, 54.7 mg, yield: 18%) as a yellow lyophilized powder. MS: m/z=531.4 [M+H]⁺. ¹H NMR (400 MHz, Dimethylsulfoxide-d₆) δ 8.12-8.03 (m, 2H), 7.97-7.94 (m, 2H), 7.45 (d, J=8.0 Hz, 2H), 7.33 (d, J=8.4 Hz, 2H), 7.07-7.02 (m, 3H), 6.67 (d, J=5.6 Hz, 1H), 6.33 (dd, J=7.6, 4.8 Hz, 1H), 3.90-3.72 (m, 1H), 3.57 (s, 2H), 2.86-2.79 (m, 2H), 2.45 (s, 3H), 2.38 (s, 3H), 2.18-2.11 (m, 2H), 1.92-1.85 (m, 2H), 1.54-1.45 (m, 2H).

### Example 413: 4-((1-(4-(2-(2-aminopyridin-3-yl)-1H-imidazo[4,5-b]pyrazin-1-yl)benzyl)piperidin-4-yl)amino)pyrimidine-2-carbonitrile

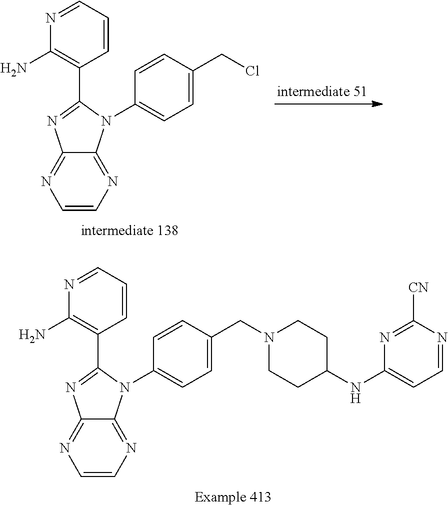 [SMILES: Nc1ncccc1-c1nc2nccnc2n1-c1ccc(CCl)cc1 ; N#Cc1nccc(NC2CCN(Cc3ccc(-n4c(-c5cccnc5N)nc5nccnc54)cc3)CC2)n1]

A mixture of Intermediate 139 (100 mg, 296 μmol) and Intermediate 51 (72.4 mg, 356 μmol) in DMF (3 mL) were added K₂CO₃ (205 mg, 1.48 mmol) and NaI (13.3 mg, 89 μmol), and then the mixture was stirred at 40° C. for 1 hr. The reaction mixture diluted with H₂O (30 mL) and extracted with EtOAc (30 mL×3). The combined organic layers were washed with H₂O (20 mL×4), dried over Na₂SO₄, filtered and concentrated under reduced pressure. After purified by silica gel flash chromatography (Eluent of 0˜5% MeOH in CH₂Cl₂), 4-((1-(4-(2-(2-aminopyridin-3-yl)-1H-imidazo[4,5-b]pyrazin-1-yl)benzyl)piperidin-4-yl)amino)pyrimidine-2-carbonitrile (Example 413, 67.4 mg, yield: 45%) was obtained as a yellow powder. MS: m/z=504.1 [M+H]⁺. ¹H NMR (400 MHz, Dimethylsulfoxide-d₆) δ 8.58 (d, J=2.4 Hz, 1H), 8.38 (d, J=2.4 Hz, 1H), 8.14-8.00 (m, 3H), 7.49-7.40 (m, 4H), 7.27 (dd, J=8.0, 1.6 Hz, 1H), 6.96 (br s, 2H), 6.67 (d, J=6.0 Hz, 1H), 6.42 (dd, J=7.6, 4.8 Hz, 1H), 3.91-3.66 (m, 1H), 3.56 (s, 2H), 2.85-2.70 (m, 2H), 2.20-2.05 (m, 2H), 1.94-1.81 (m, 2H), 1.54-1.42 (m, 2H).

### Example 414: Methyl 2-(2-aminopyridin-3-yl)-3-(4-((4-((2-cyanopyrimidin-4-yl)amino)piperidin-1-yl)methyl)phenyl)-3H-imidazo[4,5-b]pyridine-5-carboxylate

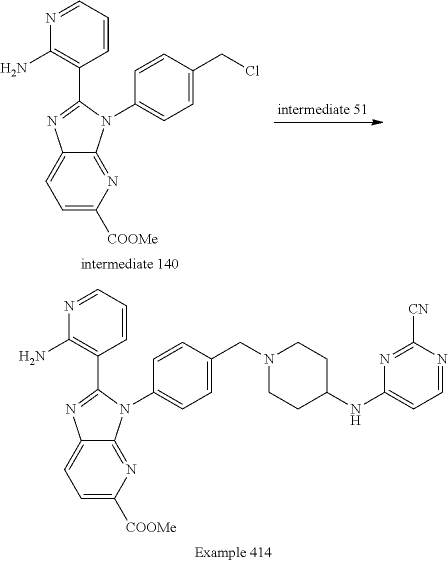 [SMILES: COC(=O)c1ccc2nc(-c3cccnc3N)n(-c3ccc(CCl)cc3)c2n1 ; COC(=O)c1ccc2nc(-c3cccnc3N)n(-c3ccc(CN4CCC(Nc5ccnc(C#N)n5)CC4)cc3)c2n1]

To a solution of Intermediate 140 (340 mg, 863 μmol) and Intermediate 51 (274 mg, 863 μmol, TFA) in DMF (5 mL) were added K₂CO₃ (597 mg, 4.32 mmol) and NaI (12.9 mg, 86.3 μmol). The mixture was stirred at 60° C. for 1 hr. The reaction mixture diluted with H₂O (10 mL) and extracted with CH₂Cl₂ (10 mL×3). The combined organic layers were washed with brine (10 mL×2), dried over Na₂SO₄, filtered and concentrated under reduced pressure. After purified by prep-HPLC (column: Waters xbridge 150*25 mm*10 μm; mobile phase: [water (NH₄HCO₃)-ACN]; gradient: 32%-62% B over 10 min), methyl 2-(2-aminopyridin-3-yl)-3-(4-((4-((2-cyanopyrimidin-4-yl)amino)piperidin-1-yl)methyl)phenyl)-3H-imidazo[4,5-b]pyridine-5-carboxylate (Example 414, 265.3 mg, 65.3 mg delivered, yield. 54%) was obtained as a yellow solid. MS: m/z=561.3 [M+H]⁺. ¹H NMR (400 MHz, Dimethylsulfoxide-d₆) δ 8.33 (d, J=8.4 Hz, 1H), 8.15-7.99 (m, 4H), 7.50-7.40 (m, 4H), 7.17 (dd, J=7.6, 2.0 Hz, 1H), 6.98 (s, 2H), 6.68 (d, J=6.0 Hz, 1H), 6.38 (dd, J=7.6, 4.8 Hz, 1H), 3.85 (s, 3H), 3.84-3.67 (m, 1H), 3.58 (s, 2H), 2.88-2.79 (m, 2H), 2.22-2.10 (m, 2H), 1.94-1.82 (m, 2H), 1.57-1.44 (m, 2H). 8%

### Example 415: 2-(2-Aminopyridin-3-yl)-3-(4-((4-((2-cyanopyrimidin-4-yl)amino)piperidin-1-yl)methyl)phenyl)-3H-imidazo[4,5-b]pyridine-5-carboxylic acid

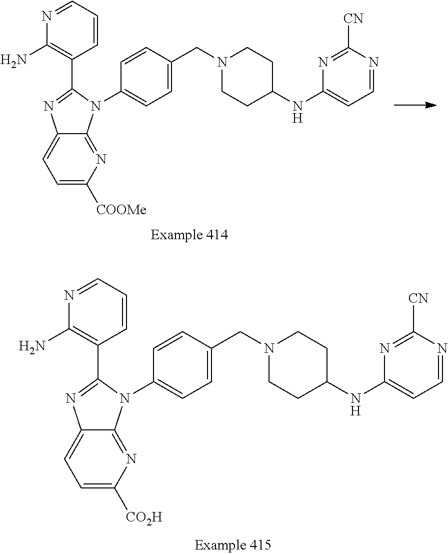 [SMILES: COC(=O)c1ccc2nc(-c3cccnc3N)n(-c3ccc(CN4CCC(Nc5ccnc(C#N)n5)CC4)cc3)c2n1 ; N#Cc1nccc(NC2CCN(Cc3ccc(-n4c(-c5cccnc5N)nc5ccc(C(=O)O)nc54)cc3)CC2)n1]

To a solution of Example 414 (200 mg, 357 μmol) in THF (2 mL) and H₂O (2 mL) was added LiOH*H₂O (15 mg, 357 μmol). After purified by prep-HPLC (column: Waters xbridge 150*25 mm*10 μm; mobile phase: [water (NH₄HCO₃)-ACN]; gradient: 11%-41% B over 10 min), 2-(2-aminopyridin-3-yl)-3-(4-((4-((2-cyanopyrimidin-4-yl)amino)piperidin-1-yl)methyl)phenyl)-3H-imidazo[4,5-b]pyridine-5-carboxylic acid (Example 415, 120 mg, yield: 44%) was obtained as a yellow solid. MS: m/z=547.4 [M+H]⁺. ¹H NMR (400 MHz, Methanol-d₄) δ 8.42 (d, J=8.4 Hz, 1H), 8.31 (d, J=8.0 Hz, 1H), 8.22-8.13 (m, 1H), 8.08 (d, J=5.2 Hz, 1H), 7.96-7.88 (m, 3H), 7.70 (d, J=7.2 Hz, 2H), 6.98-6.81 (m, 2H), 4.58-4.49 (m, 2H), 4.48-4.25 (m, 1H), 3.77-3.63 (m, 2H), 3.60-3.35 (m, 2H), 2.37-2.27 (m, 2H), 2.18-1.99 (m, 2H).

### Example 416: 4-((1-(4-(2-(2-Aminopyridin-3-yl)-5-(fluoromethoxy)-3H-imidazo[4,5-b]pyridin-3-yl)benzyl)piperidin-4-yl)amino)pyrimidine-2-carbonitrile

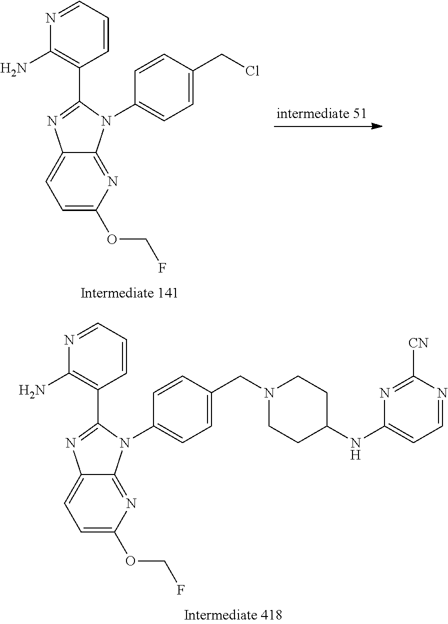 [SMILES: Nc1ncccc1-c1nc2ccc(OCF)nc2n1-c1ccc(CCl)cc1 ; N#Cc1nccc(NC2CCN(Cc3ccc(-n4c(-c5cccnc5N)nc5ccc(OCF)nc54)cc3)CC2)n1]

To a solution of Intermediate 141 (290 mg HCl salt, 690 μmol) and Intermediate 51 (240 mg TFA salt, 759 μmol) in DMF (2 mL) were added NaI (51.7 mg, 345 μmol) and K₂CO₃ (476 mg, 3.45 mmol). The mixture was stirred at 50° C. for 1 hr. The reaction mixture was diluted with water (10 mL) and extracted with EtOAc (2×10 mL). The combined organic layers were washed with brine (10 mL), dried over Na₂SO₄ filtered and concentrated under reduced pressure. After purified by silica gel flash chromatography (Eluent of 0˜4%, MeOH in CH₂Cl₂), 4-((1-(4-(2-(2-aminopyridin-3-yl)-5-(fluoromethoxy)-3H-imidazo[4,5-b]pyridin-3-yl)benzyl)piperidin-4-yl)amino)pyrimidine-2-carbonitrilecarbonitrile (Example 416, 169 mg, yield: 43%) was obtained as a yellow solid. MS: m/z=551.1 [M+H]⁺. ¹H NMR (400 MHz, Dimethylsulfoxide-d₆) δ 8.25 (d, J=8.4 Hz, 1H), 8.15-8.00 (m, 2H), 7.97 (dd, J=4.8, 1.6 Hz, 1H), 7.45 (d, J=8.0 Hz, 2H), 7.37 (d, J=8.0 Hz, 2H), 7.10 (dd, J=7.6, 2.0 Hz, 1H), 6.98 (d, J=8.8 Hz, 1H), 6.90 (br s, 2H), 6.67 (d, J=6.4 Hz, 1H), 6.36 (dd, J=7.6, 4.8 Hz, 1H), 6.10-5.92 (m, 2H), 3.88-3.73 (m, 1H), 3.57 (s, 2H), 2.81 (d, J=11.21 Hz, 2H), 2.18-2.10 (m, 2H), 1.93-1.83 (m, 2H), 1.59-1.38 (m, 2H), ¹⁹F NMR (400 MHz, Dimethylsulfoxide-d₆) δ −153.916.

### Example 417: 4-((1-(4-(2-(2-Aminopyridin-3-yl)-5-(tetrahydrofuran-3-yl)-3H-imidazo[4,5-b]pyridin-3-yl)benzyl)piperidin-4-yl)amino)pyrimidine-2-carbonitrile

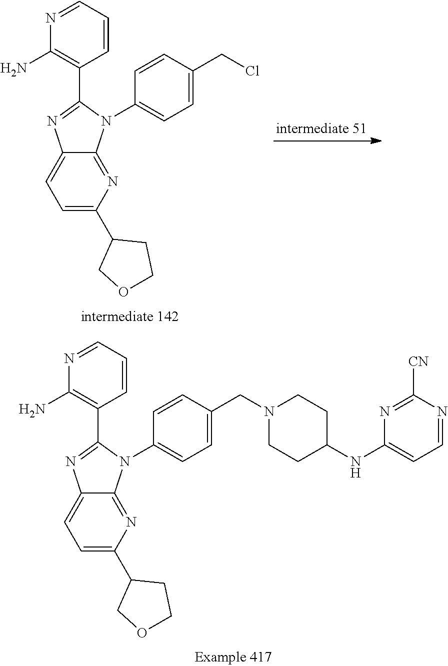 [SMILES: Nc1ncccc1-c1nc2ccc(C3CCOC3)nc2n1-c1ccc(CCl)cc1 ; N#Cc1nccc(NC2CCN(Cc3ccc(-n4c(-c5cccnc5N)nc5ccc(C6CCOC6)nc54)cc3)CC2)n1]

To a solution of Intermediate 142 (200 mg, 492 μmol) and Intermediate 51 (156 mg, 492 μmol, TFA) in DMF (4 mL) were added NaI (36.9 mg, 246 μmol) and K₂CO₃ (340 mg, 2.46 μmol). The mixture was stirred at 50° C. for 1 hr. The reaction mixture was filtered. After purified by prep-HPLC (column: [water (NH₄HCO₃)-ACN]; B %: 32%-62%, 14 min), 4-((1-(4-(2-(2-aminopyridin-3-yl)-5-(tetrahydrofuran-3-yl)-3H-imidazo[4,5-b]pyridin-3-yl)benzyl)piperidin-4-yl)amino)pyrimidine-2-carbonitrile (Example 417, 84.5 mg, yield: 26%) was obtained as a yellow solid. MS: m/z=573.4 [M+H]⁺. ¹H NMR (400 MHz, Dimethylsulfoxide-d₆) δ 8.18-8.02 (m, 3H), 7.98 (dd, J=4.8, 2.0 Hz, 1H), 7.45 (d, J=8.4 Hz, 2H), 7.40-7.33 (m, 3H), 7.11 (dd, J=8.0, 2.0 Hz, 1H), 6.96 (br s, 2H), 6.67 (d, J=6.4 Hz, 1H), 6.36 (dd, J=7.6, 4.8 Hz, 1H), 4.05 (t, J=8.0 Hz, 1H), 3.93-3.87 (m, 1H), 3.84-3.68 (m, 3H), 3.65-3.55 (m, 3H), 2.86-2.79 (m, 2H), 2.31-2.22 (m, 1H), 2.19-2.07 (m, 3H), 1.96-1.82 (m, 2H), 1.58-1.43 (m, 2H).

### Example 418: 4-((1-(4-(2-(2-Aminopyridin-3-yl)-5-(5-cyanopyridin-2-yl)-3H-imidazo[4,5-b]pyridin-3-yl)benzyl)piperidin-4-yl)amino)pyrimidine-2-carbonitrile

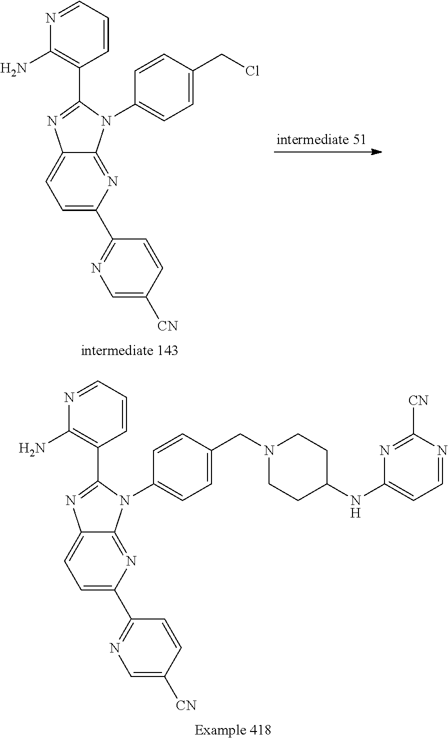 [SMILES: N#Cc1ccc(-c2ccc3nc(-c4cccnc4N)n(-c4ccc(CCl)cc4)c3n2)nc1 ; N#Cc1ccc(-c2ccc3nc(-c4cccnc4N)n(-c4ccc(CN5CCC(Nc6ccnc(C#N)n6)CC5)cc4)c3n2)nc1]

To a solution of Intermediate 143 (70 mg, 148 μmol, HCl salt) in DMF (1 mL) were added Intermediate 51 (30 mg, 148 μmol), NaI (2.21 mg, 14.8 μmol) and K₂CO₃ (61.2 mg, 443 μmol). The mixture was stirred at 50° C. for 16 hr. The reaction was concentrated under reduced pressure. After purified by prep-TLC (CH₂Cl₂:MeOH=10:1), 4-((1-(4-(2-(2-aminopyridin-3-yl)-5-(5-cyanopyridin-2-yl)-3H-imidazo[4,5-b]pyridin-3-yl)benzyl)piperidin-4-yl)amino)pyrimidine-2-carbonitrile (Example 418, 7.3 mg, yield: 8.2%) was obtained as a yellow solid. MS: m/z=605.0 [M+H]⁺. ¹H NMR (400 MHz, Chloroform-d) δ 8.90 (s, 1H), 8.59 (dd, J=8.4, 1.2 Hz, 1H), 8.44 (d, J=8.4 Hz, 1H), 8.21 (dd, J=8.4, 1.2 Hz, 1H), 8.19-8.13 (m, 1H), 8.11-8.07 (m, 1H), 7.97 (d, J=8.4 Hz, 1H), 7.61-7.54 (m, 2H), 7.42 (d, J=7.6 Hz, 2H), 7.10 (d, J=8.0 Hz, 1H), 6.67 (br s, 2H), 6.46 (d, J=6.4 Hz, 1H), 6.39-6.35 (m, 1H), 4.39-3.80 (m, 1H), 3.73 (s, 2H), 3.23-2.88 (m, 2H), 2.51-2.30 (m, 2H), 2.17-2.07 (m, 2H), 1.37-1.27 (m, 2H).

### Example 419: 4-((1-(4-(2-(2-Aminopyridin-3-yl)-5-(6-cyanopyridin-3-yl)-3H-imidazo[4,5-b]pyridin-3-yl)benzyl)piperidin-4-yl)amino)pyrimidine-2-carbonitrile

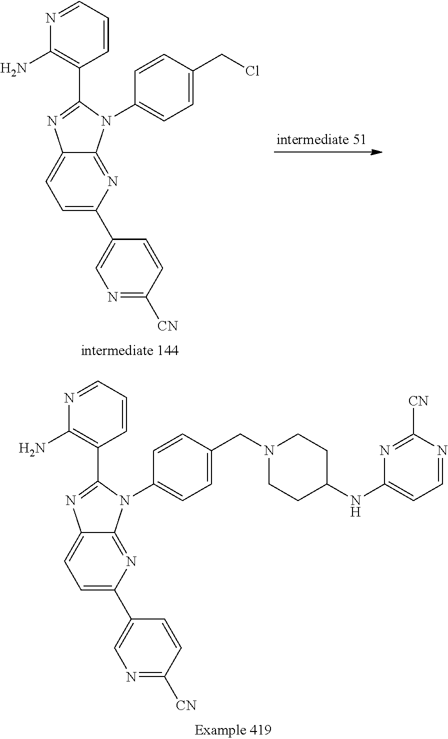 [SMILES: N#Cc1ccc(-c2ccc3nc(-c4cccnc4N)n(-c4ccc(CCl)cc4)c3n2)cn1 ; N#Cc1ccc(-c2ccc3nc(-c4cccnc4N)n(-c4ccc(CN5CCC(Nc6ccnc(C#N)n6)CC5)cc4)c3n2)cn1]

To a solution of Intermediate 144 (120 mg, 274 μmol, HCl salt) in DMF (2 mL) were added Intermediate 51 (55.7 mg, 274 μmol), NaI (8.22 mg, 54.8 μmol) and K₂CO₃ (114 mg, 822 μmol). The mixture was stirred at 50° C. for 16 hr. The mixture was filtered, and the filtrate was concentrated to give the crude product. After purified by prep-HPLC (column: Phenomenex C18 75*30 mm*3 μm; mobile phase: [water (NH₃H₂O+NH₄HCO₃)-ACN]; gradient: 47%-77% B over 7 min), 4-((1-(4-(2-(2-aminopyridin-3-yl)-5-(6-cyanopyridin-3-yl)-3H-imidazo[4,5-b]pyridin-3-yl)benzyl)piperidin-4-yl)amino)pyrimidine-2-carbonitrile (Example 419, 56.2 mg, yield: 34%) was obtained as a yellow lyophilized powder. MS: m/z=605.1 [M+H]⁺. ¹H NMR (400 MHz, Dimethylsulfoxide-d₆) δ 9.41 (d, J=2.0 Hz, 1H), 8.62 (dd, J=8.0, 2.0 Hz, 1H), 8.38 (d, J=8.4 Hz, 1H), 8.23 (d, J=8.4 Hz, 1H), 8.14 (d, J=8.4 Hz, 1H), 8.12-8.04 (m, 2H), 8.02 (dd, J=4.8, 2.0 Hz, 1H), 7.54-7.45 (m, 4H), 7.18 (dd, J=7.6, 1.6 Hz, 1H), 7.05 (br s, 2H), 6.67 (d, J=6.4 Hz, 1H), 6.40 (dd, J=7.6, 4.8 Hz, 1H), 3.90-3.73 (m, 1H), 3.59 (s, 2H), 2.88-2.78 (m, 2H), 2.23-2.09 (m, 2H), 1.94-1.82 (m, 2H), 1.56-1.44 (m, 2H).

### Example 420: 4-((1-(4-(2-(2-Aminopyridin-3-yl)-5-(oxetan-3-yl)-3H-imidazo[4,5-b]pyridin-3-yl)benzyl)piperidin-4-yl)amino)pyrimidine-2-carbonitrile

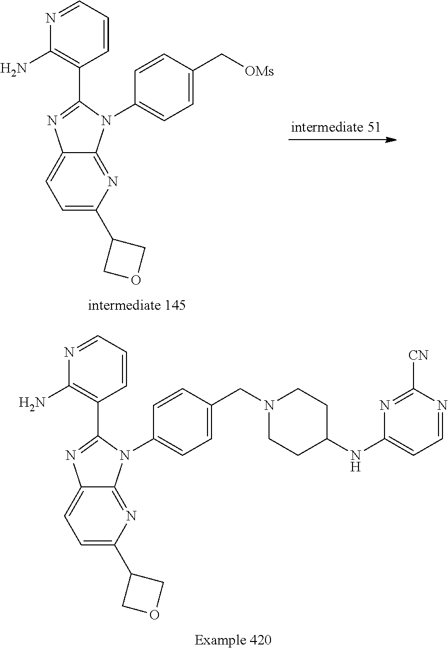 [SMILES: CS(=O)(=O)OCc1ccc(-n2c(-c3cccnc3N)nc3ccc(C4COC4)nc32)cc1 ; N#Cc1nccc(NC2CCN(Cc3ccc(-n4c(-c5cccnc5N)nc5ccc(C6COC6)nc54)cc3)CC2)n1]

To a solution of Intermediate 145 (60.5 mg, 134 pImol) in DMF (1 mL) were added DIEA (69.2 mg, 536 μmol) and Intermediate 51 (29.9 mg, 147 μmol). The mixture was stirred at 25° C. for 12 hr under N₂. After purified by prep-HPLC(column: Welch Xtimate C18 150*25 mm*5 μm; mobile phase: [water (NH₄HCO₃)-ACN]; gradient: 37%-67% B over 7 min), 4-((1-(4-(2-(2-aminopyridin-3-yl)-5-(oxetan-3-yl)-3H-imidazo[4,5-b]pyridin-3-yl)benzyl)piperidin-4-yl)amino)pyrimidine-2-carbonitrile (Example 420, 1.9 mg, yield: 2.5% for 2 steps) was obtained as yellow solid. MS: m/z=559.1 [M+H]⁺. ¹H NMR (400 MHz, Dimethylsulfoxide-d₆) δ 8.18 (d, J=6.8 Hz, 1H), 8.10-8.00 (m, 2H), 8.00-7.95 (m, 1H), 7.50-7.30 (m, 5H), 7.10 (dd, J=6.4, 1.6 Hz, 1H), 6.97 (br s, 2H), 6.68 (d, J=4.8 Hz, 1H), 6.40-6.30 (m, 1H), 4.88 (dd, J=6.8, 4.4 Hz, 2H), 4.78-4.70 (m, 2H), 4.55-4.40 (m, 1H), 3.85-3.75 (m, 1H), 3.65-3.50 (m, 2H), 2.90-2.75 (m, 2H), 2.25-2.05 (m, 2H), 1.95-1.80 (m, 2H), 1.55-1.45 (m, 2H).

### Example 421: 4-((1-(4-(2-(2-Aminopyridin-3-yl)-5-(4-methoxyphenyl)-3H-imidazo[4,5-b]pyridin-3-yl)benzyl)piperidin-4-yl)amino)pyrimidine-2-carbonitrile

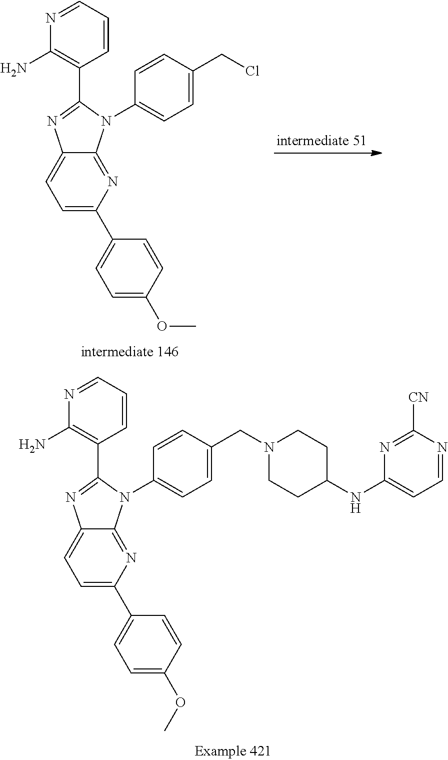 [SMILES: COc1ccc(-c2ccc3nc(-c4cccnc4N)n(-c4ccc(CCl)cc4)c3n2)cc1 ; COc1ccc(-c2ccc3nc(-c4cccnc4N)n(-c4ccc(CN5CCC(Nc6ccnc(C#N)n6)CC5)cc4)c3n2)cc1]

To a solution of Intermediate 146 (200 mg, 453 μmol) in DMF (3 mL) were added DIEA (234 mg, 1.81 mmol) and Intermediate 51 (101 mg, 498 μmol). The mixture was stirred at 80° C. for 2 hr under N₂. After purified byprep-HPLC(column: Phenomenex C18 80*40 mm*3 μm; mobile phase: [water (NH₃H₂O+NH₄HCO₃)-ACN]; gradient: 62%-92% B over 7 min), 4-((1-(4-(2-(2-aminopyridin-3-yl)-5-(4-methoxyphenyl)-3H-imidazo[4,5-b]pyridin-3-yl)benzyl)piperidin-4-yl)amino)pyrimidine-2-carbonitrile (Example 421, 41.3 mg, yield: 15%) was obtained as a yellow solid. MS: m/z=609.2 [M+H]⁺. ¹H NMR (400 MHz, Dimethylsulfoxide-d₆) δ 8.22 (d, J=8.0 Hz, 1H), 8.08 (d, J=6.0 Hz, 2H), 8.03-7.90 (m, 3H), 7.91 (d, J=8.8 Hz, 1H), 7.50-7.40 (m, 4H), 7.18-7.00 (m, 5H), 6.68 (d, J=6.0 Hz, 1H), 6.40-6.30 (m, 1H), 4.00-3.70 (m, 4H), 3.59 (s, 2H), 2.83 (d, J=10.8 Hz, 2H), 2.30-2.10 (m, 2H), 2.00-1.80 (m, 2H), 1.60-1.40 (m, 2H).

### Example 422: 4-((1-(4-(2-(2-Aminopyridin-3-yl)-5-(2-methoxyphenyl)-3H-imidazo[4,5-b]pyridin-3-yl)benzyl)piperidin-4-yl)amino)pyrimidine-2-carbonitrile

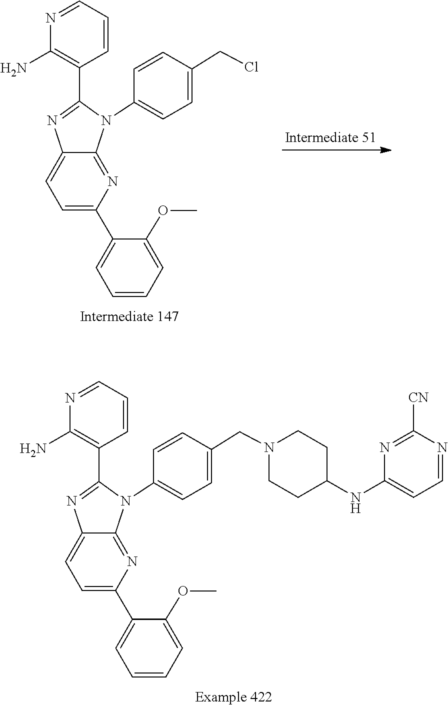 [SMILES: COc1ccccc1-c1ccc2nc(-c3cccnc3N)n(-c3ccc(CCl)cc3)c2n1 ; COc1ccccc1-c1ccc2nc(-c3cccnc3N)n(-c3ccc(CN4CCC(Nc5ccnc(C#N)n5)CC4)cc3)c2n1]

To a solution of Intermediate 147 (320 mg, 724 μmol) and Intermediate 51 (147 mg, 724 μmol) in DMF (3 mL) were added NaI (21.7 mg, 145 μmol) and K₂CO₃ (400 mg, 2.90 mmol). The mixture was stirred at 50° C. for 1 hr. The mixture was diluted with water (10 mL) and extracted with CH₂Cl₂ (20 mL×3). The combined organic layers were washed with brine (20 mL×2), dried over Na₂SO₄, filtered and concentrated under reduced pressure. After purified by silica gel flash chromatography (Eluent of 4˜5% MeOH in CH₂Cl₂), 4-((1-(4-(2-(2-aminopyridin-3-yl)-5-(2-methoxyphenyl)-3H-imidazo[4,5-b]pyridin-3-yl)benzyl)piperidin-4-yl)amino)pyrimidine-2-carbonitrile (Example 422, 35 mg, yield: 7.7%) was obtained as a yellow lyophilized powder. MS: m/z=609.1 [M+H]⁺. ¹H NMR (400 MHz, Methanol-d₄) δ 8.14 (d, J=8.4 Hz, 1H), 8.03-8.01 (m, 1H), 7.99-7.95 (m, 1H), 7.90 (d, J=8.8 Hz, 1H), 7.64 (dd, J=7.2 Hz, 1.2 Hz, 1H), 7.55 (d, J=7.2 Hz, 2H), 7.47 (d, J=8.0 Hz, 2H), 7.38-7.32 (m, 2H), 7.11 (d, J=8.4 Hz, 1H), 7.00 (t, J=7.2 Hz, 1H), 6.61-6.58 (m, 1H), 6.48 (dd, J=7.2, 5.2 Hz, 1H), 4.06-3.94 (m, 1H), 3.86 (s, 3H), 3.83-3.73 (m, 2H), 3.09-3.02 (m, 2H), 2.54-2.31 (m, 2H), 2.10-2.01 (m, 2H), 1.66-1.58 (m, 2H).

### Example 423: 4-((1-(4-(2-(2-Aminopyridin-3-yl)-5-(trifluoromethoxy)-3H-imidazo[4,5-b]pyridin-3-yl)benzyl)piperidin-4-yl)amino)pyrimidine-2-carbonitrile

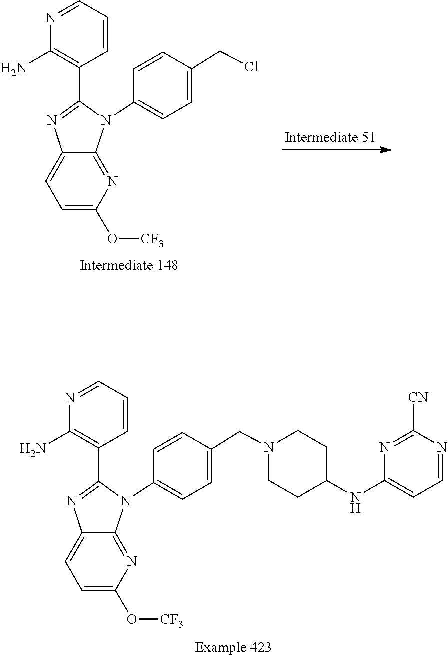 [SMILES: Nc1ncccc1-c1nc2ccc(OC(F)(F)F)nc2n1-c1ccc(CCl)cc1 ; N#Cc1nccc(NC2CCN(Cc3ccc(-n4c(-c5cccnc5N)nc5ccc(OC(F)(F)F)nc54)cc3)CC2)n1]

To a solution of Intermediate 148 (150 mg, 357 μmol) and Intermediate 51 (79.9 mg, μmol) in DMF (3 mL) were added K₂CO₃ (148 mg, 1.07 mmol) and NaI (10.7 mg, 71.5 μmol) at 25° C. This mixture was stirred at 80° C. for 2 hr. The mixture was quenched with H₂O (20 mL) and extracted with EtOAc (20 mL×3). The combined organic phase was filtered and concentrated to give the residue. After by prep-HPLC (column: Phenomenex C18 75×30 mm×3 μm; mobile phase: [water (NH₃H₂O+NH₄HCO₃)-ACN]; gradient: 47%-77% B over 7 min), 4-((1-(4-(2-(2-aminopyridin-3-yl)-5-(trifluoromethoxy)-3H-imidazo[4,5-b]pyridin-3-yl)benzyl)piperidin-4-yl)amino)pyrimidine-2-carbonitrile (Example 423, 40.9 mg, yield: 20% for 2 steps) was obtained as a light yellow solid. MS: m/z=587.1 [M+H]r. ¹H NMR (400 MHz, Dimethylsulfoxide-d₆) δ 8.41 (d, J=8.4 Hz, 1H), 8.12-7.98 (m, 3H), 7.45 (d, J=8.4 Hz, 2H), 7.38 (d, J=8.0 Hz, 2H), 7.28 (d, J=8.4 Hz, 1H), 7.18 (dd, J=7.6, 1.6 Hz, 1H), 6.85 (s, 2H), 6.67 (d, J=6.0 Hz, 1H), 6.39 (dd, J=7.6, 4.8 Hz, 1H), 3.88-3.74 (m, 1H), 3.56 (s, 2H), 2.83-2.74 (m, 2H), 2.18-2.09 (m, 2H), 1.94-1.82 (m, 2H), 1.56-1.44 (m, 2H). ¹⁹F NMR (400 MHz, Dimethylsulfoxide-d₆) δ −55.510.

### Example 424: 4-(4-(4-(2-(2-Aminopyridin-3-yl)-5-(2-cyanopyridin-3-yl)-3H-imidazo[4,5-b]pyridin-3-yl)benzyl)piperazin-1-yl)pyrimidine-2-carbonitrile

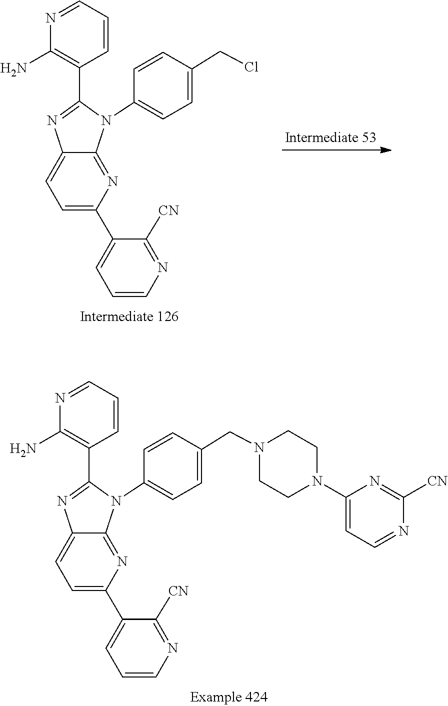 [SMILES: N#Cc1ncccc1-c1ccc2nc(-c3cccnc3N)n(-c3ccc(CCl)cc3)c2n1 ; N#Cc1nccc(N2CCN(Cc3ccc(-n4c(-c5cccnc5N)nc5ccc(-c6cccnc6C#N)nc54)cc3)CC2)n1]

To a solution of Intermediate 126 (200 mg HCl salt, 457 μmol) and Intermediate 53 (138 mg TFA salt, 457 μmol) in DMF (2 mL) was added NaI (34 mg, 228 μmol) and K₂CO₃ (315 mg, 2.28 μmol). The mixture was stirred at 50° C. for 1 hr. The reaction mixture was filtered and the residue was purified by prep-HPLC (column. [water (NH₄HCO₃)-ACN]; B %: 36%-66%, 14 min), 4-(4-(4-(2-(2-aminopyridin-3-yl)-5-(2-cyanopyridin-3-yl)-3H-imidazo[4,5-b]pyridin-3-yl)benzyl)piperazin-1-yl)pyrimidine-2-carbonitrile (Example 424, 32 mg, yield: 29%) was obtained as a purple solid. MS: m/z=591.4 [M+H]⁺. ¹H NMR (400 MHz, Dimethylsulfoxide-d₆) δ 8.79 (dd, J=4.8, 1.2 Hz, 1H), 8.43 (d, J=8.0 Hz, 1H), 8.37 (dd, J=8.0, 1.2 Hz, 1H), 8.26 (d, J=6.4 Hz, 1H), 8.02 (dd, J=4.8, 1.6 Hz, 1H), 7.94 (d, J=8.4 Hz, 1H), 7.85 (dd, J=8.0, 4.8 Hz, 1H), 7.51-7.46 (m, 4H), 7.21 (dd, J=7.6, 1.6 Hz, 1H), 7.11 (d, J=6.8 Hz, 1H), 7.01 (br s, 2H), 6.40 (dd, J=7.6, 4.4 Hz, 1H), 3.74-3.64 (m, 4H), 3.61 (s, 2H), 2.49-2.43 (m, 4H).

### Example 425: 4-((1-(4-(2-(2-Aminopyridin-3-yl)-5-isopropoxy-3H-imidazo[4,5-b]pyridin-3-yl)benzyl)piperidin-4-yl)amino)pyrimidine-2-carbonitrile

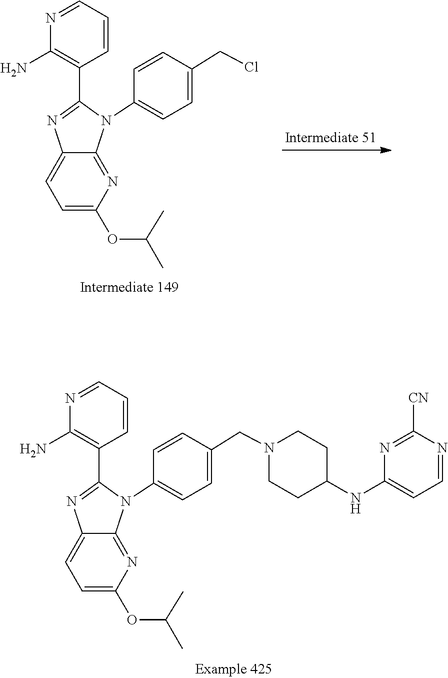 [SMILES: CC(C)Oc1ccc2nc(-c3cccnc3N)n(-c3ccc(CCl)cc3)c2n1 ; CC(C)Oc1ccc2nc(-c3cccnc3N)n(-c3ccc(CN4CCC(Nc5ccnc(C#N)n5)CC4)cc3)c2n1]

To a mixture of Intermediate 149 (200 mg, 508 μmol) and Intermediate 51 (103 mg, 508 μmol) in DMF (2 mL) was added DIEA (354 μL, 2.03 mmol). The mixture was stirred at 80° C. for 1 hr. The mixture was concentrated. After purified by prep-HPLC (column: Phenomenex C18 80*40 mm*3 μm; mobile phase: [water (NH₃H₂O+NH₄HCO₃)-ACN]; gradient: 55%-85% B over 7 min), 4-((1-(4-(2-(2-aminopyridin-3-yl)-5-isopropoxy-3H-imidazo[4,5-b]pyridin-3-yl)benzyl)piperidin-4-yl)amino)pyrimidine-2-carbonitrile (Example 425, 44.4 mg, yield: 16%) was obtained as a yellow solid. MS: m/z=561.1 [M+H]⁺. ¹H NMR (400 MHz, Dimethylsulfoxide-d₆) δ 8.20-8.02 (m, 3H), 7.95 (dd, J=4.8, 1.6 Hz, 1H), 7.47-7.41 (m, 2H), 7.38-7.31 (m, 2H), 7.06 (dd, J=7.6, 1.6 Hz, 1H), 6.97 (br s, 2H), 6.73 (d, J=8.4 Hz, 1H), 6.67 (d, J=6.0 Hz, 1H), 6.34 (dd, J=7.6, 4.8 Hz, 1H), 5.08-4.98 (m, 1H), 3.93-3.72 (m, 1H), 3.57 (s, 2H), 2.85-2.74 (m, 2H), 2.22-2.07 (m, 2H), 1.96-1.78 (m, 2H), 1.56-1.41 (m, 2H), 1.24 (d, J=6.4 Hz, 6H).

### Example 426: 4-((4-(4-(2-(2-Aminopyridin-3-yl)-5-phenyl-3H-imidazo[4,5-b]pyridin-3-yl)benzyl)piperazin-1-yl)amino)pyrimidine-2-carbonitrile

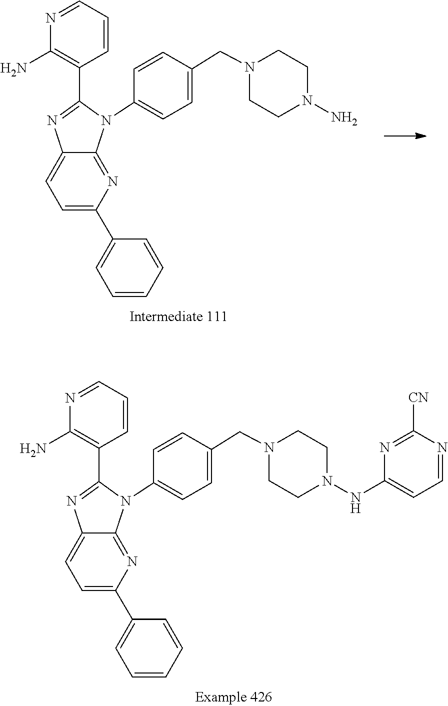 [SMILES: Nc1ncccc1-c1nc2ccc(-c3ccccc3)nc2n1-c1ccc(CN2CCN(N)CC2)cc1 ; N#Cc1nccc(NN2CCN(Cc3ccc(-n4c(-c5cccnc5N)nc5ccc(-c6ccccc6)nc54)cc3)CC2)n1]

A mixture of Intermediate 111 (100 mg HCl salt, 195 μmol), 4-chloropyrimidine-2-carbonitrile (27.2 mg, 195 μmol), and DIEA (126 mg, 975 μmol, 170 μL) in NMP (3 mL) was taken up into a microwave tube. The sealed tube was heated at 160° C. for 1 hr under microwave. The crude was diluted with CH₂Cl₂ (5 mL) and H₂O (5 mL) and extracted with CH₂Cl₂ (15 mL×3). The combined organic layers were washed with brine (5 mL), dried over anhydrous Na₂SO₄, filtered and concentrated. The residue was purified by prep-HPLC (column: Phenomenex C18 75×30 mm×3 μm; mobile phase: [water (NH₃H₂O+NH₄HCO₃)-ACN]; gradient: 50%-80% B over 8 min) to give 4-((4-(4-(2-(2-aminopyridin-3-yl)-5-phenyl-3H-imidazo[4,5-b]pyridin-3-yl)benzyl)piperazin-1-yl)amino)pyrimidine-2-carbonitrile (Example 426, 3 mg, yield: 2.6%) as a light-yellow solid. MS: m/z=580.4 [M+H]⁺. ¹H NMR (400 MHz, Dimethylsulfoxide-d₆) δ 9.34 (s, 1H), 8.32-8.22 (m, 2H), 8.06-7.95 (m, 4H), 7.52-7.42 (m, 6H), 7.45-7.36 (m, 1H), 7.16 (dd, J=7.6, 1.6 Hz, 1H), 7.03 (br s, 2H), 7.01-6.97 (m, 1H), 6.38 (dd, J=8.0, 4.8 Hz, 1H), 3.61 (s, 2H), 3.32-3.31 (m, 2H), 2.98-2.66 (m, 6H).

### Example 427: 4-((1-(4-(2-(2-Aminopyridin-3-yl)-5-(methoxy-d₃)-3H-imidazo[4,5-b]pyridin-3-yl)benzyl)piperidin-4-yl)amino)pyrimidine-2-carbonitrile

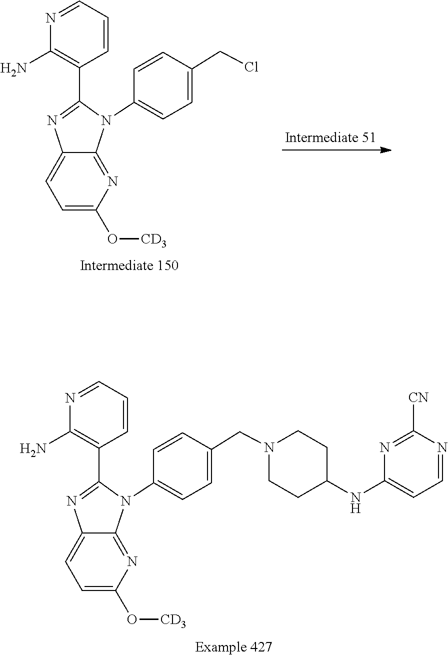 [SMILES: [2H]C([2H])([2H])Oc1ccc2nc(-c3cccnc3N)n(-c3ccc(CCl)cc3)c2n1 ; [2H]C([2H])([2H])Oc1ccc2nc(-c3cccnc3N)n(-c3ccc(CN4CCC(Nc5ccnc(C#N)n5)CC4)cc3)c2n1]

To a solution of Intermediate 150 (105 mg, 285 μmol) and Intermediate 51 (63.8 mg, 314 μmol) in DMF (3 mL) were added K₂CO₃ (118 mg, 856 μmol) and NaI (8.56 mg, 57.1 μmol) in one portion at 20° C. The reaction mixture was stirred at 50° C. for 2 hr. H₂O (10 mL) was added at 20° C. and the mixture was extracted with EtOAc (10 mL×3). The combined organic layers were washed with brine (10 mL×2), dried over anhydrous Na₂SO₄, filtered and concentrated under reduced pressure. The residue purified by prep-HPLC(column: Welch Xtimate C18 150×25 mm×5 μm; mobile phase: [water (NH₄HCO₃)-ACN]; gradient: 45%-75% B over 7 min) to give 4-((1-(4-(2-(2-aminopyridin-3-yl)-5-(methoxy-d₃)-3H-imidazo[4,5-b]pyridin-3-yl)benzyl)piperidin-4-yl)amino)pyrimidine-2-carbonitrile (Example 427, 37.5 mg, yield: 12%) as an off-white solid. MS: m/z=536.1 [M+H]⁺. ¹H NMR (400 MHz, Dimethylsulfoxide-d₆) δ 8.13-8.04 (m, 3H), 7.95 (dd, J=4.8, 1.6 Hz, 1H), 7.46-7.42 (m, 2H), 7.39-7.35 (m, 2H), 7.04 (dd, J=7.6, 1.6 Hz, 1H), 6.96 (s, 2H), 6.81 (d, J=8.4 Hz, 1H), 6.67 (d, J=6.0 Hz, 1H), 6.34 (dd, J=7.6, 4.8 Hz, 1H), 3.81-3.79 (m, 1H), 3.57 (s, 2H), 2.84-2.71 (m, 2H), 2.18-2.09 (m, 2H), 1.92-1.84 (m, 2H), 1.52-1.43 (m, 2H).

### Example 428: 4-((1-(4-(2-(2-Aminopyridin-3-yl)-5-(6-cyanopyridin-2-yl)-3H-imidazo[4,5-b]pyridin-3-yl)benzyl)piperidin-4-yl)amino)pyrimidine-2-carbonitrile

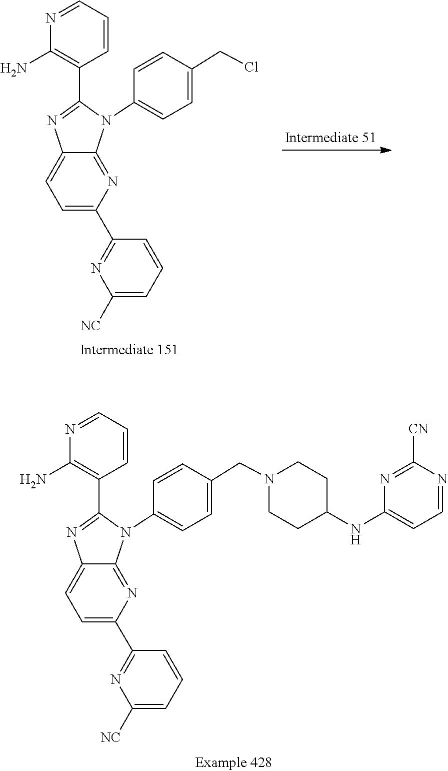 [SMILES: N#Cc1cccc(-c2ccc3nc(-c4cccnc4N)n(-c4ccc(CCl)cc4)c3n2)n1 ; N#Cc1cccc(-c2ccc3nc(-c4cccnc4N)n(-c4ccc(CN5CCC(Nc6ccnc(C#N)n6)CC5)cc4)c3n2)n1]

To a mixture of Intermediate 151 (320 mg, 730 μmol) and Intermediate 51 (148 mg, 731 μmol) in DMF (5 mL) was added DIEA (509 μL, 2.92 mmol). The mixture was stirred at 80° C. for 1 hr. H₂O (50 mL) was added, and the aqueous layer was extracted with EtOAc (30 mL×2). The combined organic layers were washed with brine (20 mL), dried over Na₂SO₄, filtered and concentrated. After purified by prep-HPLC (column: Phenomenex C18 75*30 mm*3 μm; mobile phase: [water (NH₃H₂O+NH₄HCO₃)-ACN]; gradient: 45%-75% B over 7 min), 4-((1-(4-(2-(2-aminopyridin-3-yl)-5-(6-cyanopyridin-2-yl)-3H-imidazo[4,5-b]pyridin-3-yl)benzyl)piperidin-4-yl)amino)pyrimidine-2-carbonitrile (Example 428, 170.3 mg, yield: 39% for 2 steps) was obtained as a yellow solid. MS: m/z=605.1 [M+H]⁺. ¹H NMR (400 MHz, Dimethylsulfoxide-d₆) δ 8.48-8.40 (m, 2H), 8.38-8.33 (m, 1H), 8.26-7.97 (m, 5H), 7.56-7.43 (m, 4H), 7.18 (dd, J=7.6, 1.6 Hz, 1H), 7.03 (s, 2H), 6.67 (d, J=6.0 Hz, 1H), 6.39 (dd, J=8.0, 5.2 Hz, 1H), 3.91-3.73 (m, 1H), 3.50 (s, 2H), 2.88-2.79 (m, 2H), 2.20-2.10 (m, 2H), 1.94-1.81 (m, 2H), 1.56-1.39 (m, 2H).

### Example 429: 4-((1-(4-(2-(2-Aminopyridin-3-yl)-5-(2-cyanopyridin-4-yl)-3H-imidazo[4,5-b]pyridin-3-yl)benzyl)piperidin-4-yl)amino)pyrimidine-2-carbonitrile

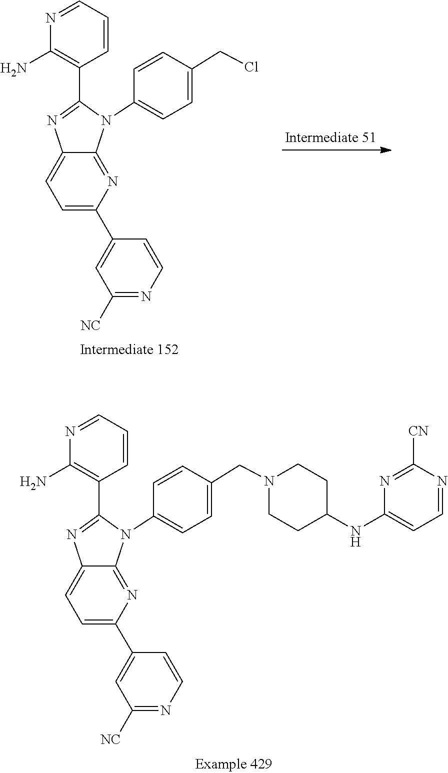 [SMILES: N#Cc1cc(-c2ccc3nc(-c4cccnc4N)n(-c4ccc(CCl)cc4)c3n2)ccn1 ; N#Cc1cc(-c2ccc3nc(-c4cccnc4N)n(-c4ccc(CN5CCC(Nc6ccnc(C#N)n6)CC5)cc4)c3n2)ccn1]

To a solution of Intermediate 152 (400 mg, 913 μmol) and Intermediate 51 (204 mg, 1.00 μmol) in DMF (5 mL) were added K₂CO₃ (379 mg, 2.74 mmol) and NaI (13.7 mg, 91.4 μmol) at 25° C. This mixture was stirred at 80° C. for 1 hr. The mixture was quenched with H₂O (50 mL) and then extracted with EtOAc (30 mL×3). The combined organic phase was filtered and concentrated. After purified by prep-HPLC (column: Welch Xtimate C18 150*25 mm*5 μm; mobile phase: [water (NH₄HCO₃)-ACN]; gradient: 50%-80% B over 7 min), 4-((1-(4-(2-(2-aminopyridin-3-yl)-5-(2-cyanopyridin-4-yl)-3H-imidazo[4,5-b]pyridin-3-yl)benzyl)piperidin-4-yl)amino)pyrimidine-2-carbonitrile (Example 429, 370 mg, yield: 67%) was obtained as a yellow solid. MS: m/z=605.2 [M+H]⁺. ¹H NMR (400 MHz, Dimethylsulfoxide-d₆) 8.82 (d, J=5.2 Hz, 1H), 8.62 (s, 1H), 8.39 (d, J=8.0 Hz, 1H), 8.32 (dd, J=4.8, 1.6 Hz, 1H), 8.28 (d, J=8.4 Hz, 1H), 8.11-8.04 (m, 2H), 8.02 (dd, J=4.8, 1.6 Hz, 1H), 7.52-7.45 (m, 4H), 7.18 (dd, J=7.6, 1.6 Hz, 1H), 7.06 (s, 2H), 6.67 (d, J=6.0 Hz, 1H), 6.39 (dd, J=7.6, 4.8 Hz, 1H), 3.87-3.74 (m, 1H), 3.60 (s, 2H), 2.89-2.80 (m, 2H), 2.20-2.11 (m, 2H), 1.95-1.82 (m, 2H), 1.58-1.43 (m, 2H).

### Example 430: 4-((1-(4-(2-(2-Aminopyridin-3-yl)-5-(5-cyanopyridin-3-yl)-3H-imidazo[4,5-b]pyridin-3-yl)benzyl)piperidin-4-yl)amino)pyrimidine-2-carbonitrile

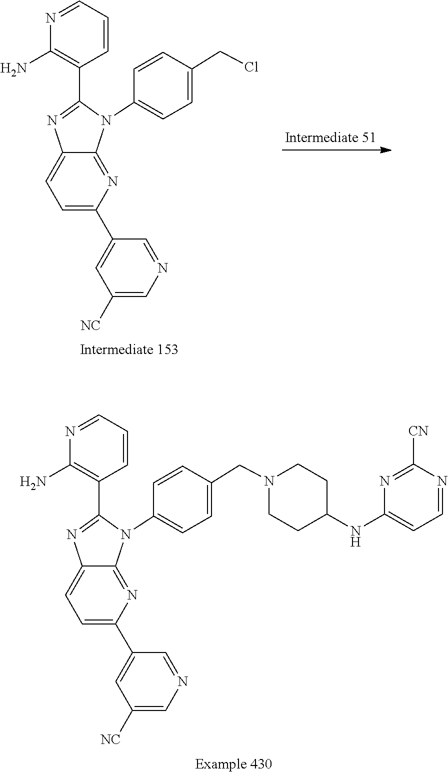 [SMILES: N#Cc1cncc(-c2ccc3nc(-c4cccnc4N)n(-c4ccc(CCl)cc4)c3n2)c1 ; N#Cc1cncc(-c2ccc3nc(-c4cccnc4N)n(-c4ccc(CN5CCC(Nc6ccnc(C#N)n6)CC5)cc4)c3n2)c1]

To a solution of Intermediate 153 (240 mg, 548 μmol) and Intermediate 51 (134 mg, 658 μmol) in DMF (8 mL) were added NaI (16.4 mg, 110 μmol) and K₂CO₃ (227 mg, 1.64 mmol) at 20° C. The reaction mixture was stirred at 80° C. for 16 hr. H₂O (10 mL) was added at 20° C. and the mixture was extracted with EtOAc (10 mL×3). The combined organic layers were washed with water (10 mL×2), dried over anhydrous Na₂SO₄, filtered and concentrated under reduced pressure. After purified by prep-HPLC (column: Boston Prime C18 150×30 mm×5 μm; mobile phase: [water (NH₃H₂O+NH₄HCO₃)-ACN]; gradient: 52%-82% B over 7 min), 4-((1-(4-(2-(2-aminopyridin-3-yl)-5-(5-cyanopyridin-3-yl)-3H-imidazo[4,5-b]pyridin-3-yl)benzyl)piperidin-4-yl)amino)pyrimidine-2-carbonitrile (Example 430, 90.8 mg, yield: 12.9% for 4 steps) was obtained as an off-white solid MS: m/z=605.3 [M+H]⁺. ¹H NMR (400 MHz, Dimethylsulfoxide-d₆) δ 9.48 (d, J=2.0 Hz, 1H), 9.03 (s, 1H), 8.86 (s, 1H), 8.38 (d, J=8.4 Hz, 1H), 8.18 (d, J=8.4 Hz, 1H), 8.13-7.99 (m, 3H), 7.54-7.45 (m, 4H), 7.18 (d, J=6.8 Hz, 1H), 7.06 (s, 2H), 6.67 (d, J=6.0 Hz, 1H), 6.40 (dd, J=7.6, 4.8 Hz, 1H), 3.82-3.81 (m, 1H), 3.60 (s, 2H), 2.89-2.76 (m, 2H), 2.21-2.09 (m, 2H), 1.94-1.81 (m, 2H), 1.61-1.43 (m, 2H).

### Example 431: 4-((1-(4-(2-(2-Aminopyridin-3-yl)-5-(pyridin-3-yl)-3H-imidazo[4,5-b]pyridin-3-yl)benzyl)piperidin-4-yl)amino)pyrimidine-2-carbonitrile

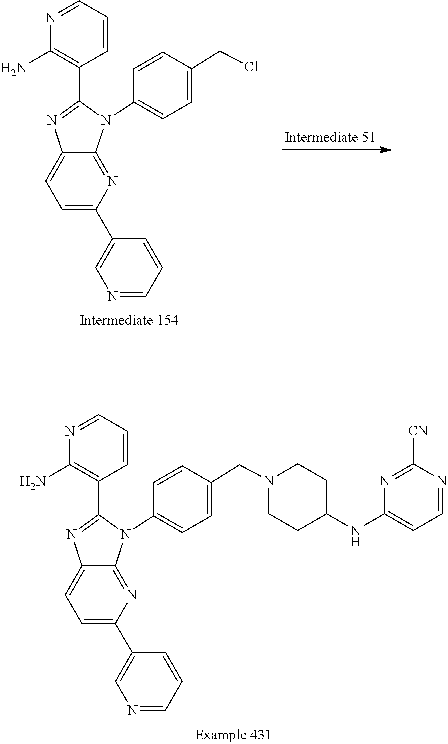 [SMILES: Nc1ncccc1-c1nc2ccc(-c3cccnc3)nc2n1-c1ccc(CCl)cc1 ; N#Cc1nccc(NC2CCN(Cc3ccc(-n4c(-c5cccnc5N)nc5ccc(-c6cccnc6)nc54)cc3)CC2)n1]

To a solution of Intermediate 154 (210 mg, 508 μmol) and Intermediate 51 (113 mg, 559 μmol) in DMF (3 mL) was added DIEA (263 mg, 2.03 mmol). The mixture was stirred at 80° C. for 2 hr under N₂. The mixture was concentrated and purified by prep-HPLC(column: Phenomenex C18 80*40 mm*3 μm; mobile phase: [water (NH₃H₂O+NH₄HCO₃)-ACN]; gradient: 50%-80% B over 7 min) to give 4-((1-(4-(2-(2-aminopyridin-3-yl)-5-(pyridin-3-yl)-3H-imidazo[4,5-b]pyridin-3-yl)benzyl)piperidin-4-yl)amino)pyrimidine-2-carbonitrile (Example 431, 37.7 mg, yield: 13%) as a yellow solid. MS: m/z=580.3 [M+H]⁺. ¹H NMR (400 MHz, Dimethylsulfoxide-d₆) δ 9.23 (d, J=1.6 Hz, 1H), 8.70-8.50 (m, 1H), 8.40-8.25 (m, 2H), 8.20-7.90 (m, 4H), 7.60-7.40 (m, 5H), 7.30-7.10 (m, 1H), 7.10-6.90 (m, 1H), 6.67 (d, J=6.4 Hz, 1H), 6.38 (dd, J=7.2, 4.8 Hz, 1H), 3.81-3.80 (m, 1H), 3.59 (s, 2H), 2.83-2.81 (m, 2H), 2.25-2.05 (m, 2H), 1.95-1.70 (m, 2H), 1.60-1.40 (m, 2H).

### Example 432: 4-(1-(4-(2-(2-Aminopyridin-3-yl)-5-(pyridin-3-yl)-3H-imidazo[4,5-b]pyridin-3-yl)benzyl)piperidin-4-yl)pyrimidine-2-carbonitrile

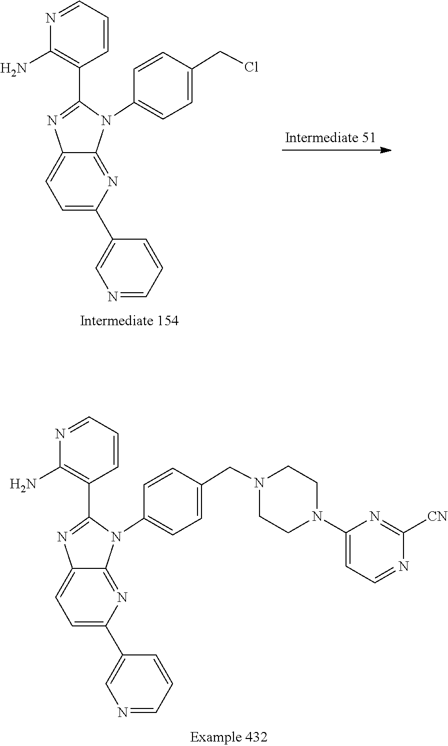 [SMILES: Nc1ncccc1-c1nc2ccc(-c3cccnc3)nc2n1-c1ccc(CCl)cc1 ; N#Cc1nccc(N2CCN(Cc3ccc(-n4c(-c5cccnc5N)nc5ccc(-c6cccnc6)nc54)cc3)CC2)n1]

To a solution of Intermediate 154 (170 mg, 412 μmol) and Intermediate 53 (85.7 mg, 453 μmol) in DMF (3 mL) was added DIEA (213 mg, 1.65 mmol). The mixture was stirred at 80° C. for 2 hr under N₂. After purified by prep-HPLC(column: Boston Prime C18 150*30 mm*5 μm; mobile phase: [water (NH₃H₂O+NH₄HCO₃)-ACN]; gradient: 45%-75% B over 7 min), 4-(1-(4-(2-(2-aminopyridin-3-yl)-5-(pyridin-3-yl)-3H-imidazo[4,5-b]pyridin-3-yl)benzyl)piperidin-4-yl)pyrimidine-2-carbonitrile (Example 432, 15.1 mg, yield: 6.5%) was obtained as a yellow solid. MS: m/z=566.3 [M+H]⁺. ¹H NMR (400 MHz, Dimethylsulfoxide-d₆) δ 9.23 (br s, 1H), 8.59 (d, J=2.8 Hz, 1H), 8.40-8.20 (m, 3H), 8.10-7.90 (m, 2H), 7.60-7.40 (m, 5H), 7.25-7.00 (m, 4H), 6.50-6.30 (m, 1H), 4.00-3.50 (m, 8H), 3.30-3.10 (m, 2H).

### Example 433: 4-((1-(4-(2-(2-Aminopyridin-3-yl)-5-(pyridin-4-yl)-3H-imidazo[4,5-b]pyridin-3-yl)benzyl)piperidin-4-yl)amino)pyrimidine-2-carbonitrile

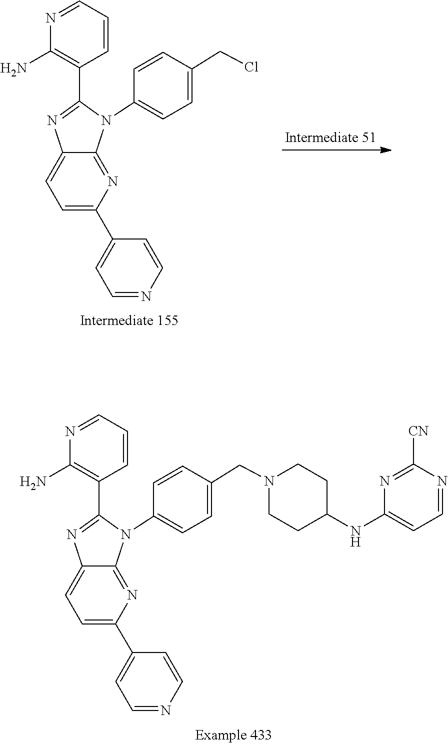 [SMILES: Nc1ncccc1-c1nc2ccc(-c3ccncc3)nc2n1-c1ccc(CCl)cc1 ; N#Cc1nccc(NC2CCN(Cc3ccc(-n4c(-c5cccnc5N)nc5ccc(-c6ccncc6)nc54)cc3)CC2)n1]

To a solution of Intermediate 155 (104 mg, 253 μmol) and Intermediate 51 (72.0 mg, 354 μmol) in DMF (1 mL) was added DIEA (130 mg, 1.01 mmol). The mixture was stirred at 25° C. for 12 hr. After purified by prep-HPLC (column: Welch Xtimate C18 150*25 mm*5 μm; mobile phase: [water (NH₄HCO₃)-ACN]; gradient: 40%-70% B over 7 min), 4-((1-(4-(2-(2-aminopyridin-3-yl)-5-(pyridin-4-yl)-3H-imidazo[4,5-b]pyridin-3-yl)benzyl)piperidin-4-yl)amino)pyrimidine-2-carbonitrile (Example 433, 58.4 mg, yield: 40%) was obtained as a light yellow solid. MS: m/z=580.4 [M+H]⁺. ¹H NMR (400 MHz, Dimethylsulfoxide-d₆) 8.68-8.64 (m, 2H), 8.35 (d, J=8.4 Hz, 1H), 8.16 (d, J=8.8 Hz, 1H), 8.11-8.05 (m, 2H), 8.03-7.99 (m, 3H), 7.52-7.45 (m, 4H), 7.18 (dd, J=7.6, 2.0 Hz, 1H), 7.04 (br s, 2H), 6.68 (d, J=6.4 Hz, 1H), 6.39 (dd, J=8.0, 4.8 Hz, 1H), 3.90-3.73 (m, 1H), 3.60 (s, 2H), 2.84-2.82 (m, 2H), 2.22-2.09 (m, 2H), 1.95-1.81 (m, 2H), 1.57-1.43 (m, 2H).

### Example 434: 4-((7-(4-(2-(2-Aminopyridin-3-yl)-5-morpholino-3H-imidazo[4,5-b]pyridin-3-yl)benzyl)-7-azaspiro[3.5]nonan-2-yl)amino)-1,3,5-triazine-2-carbonitrile

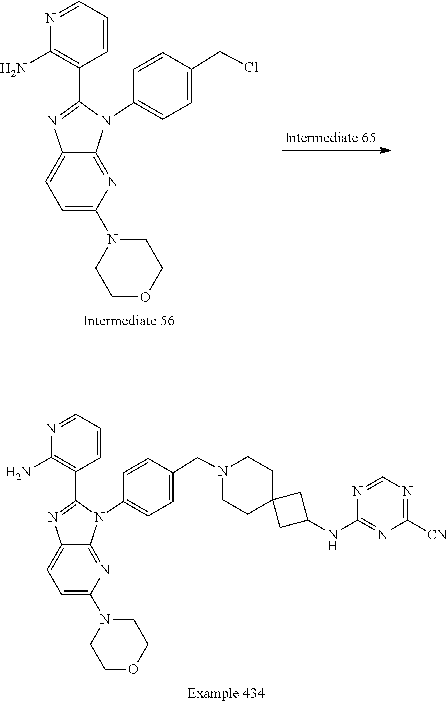 [SMILES: Nc1ncccc1-c1nc2ccc(N3CCOCC3)nc2n1-c1ccc(CCl)cc1 ; N#Cc1ncnc(NC2CC3(CCN(Cc4ccc(-n5c(-c6cccnc6N)nc6ccc(N7CCOCC7)nc65)cc4)CC3)C2)n1]

To a solution of Intermediate 56 (200 mg, 475 μmol) and Intermediate 65 (139 mg, 570 μmol) in DMF (5 mL) was added NaI (71 mg, 475 μmol) and K₂CO₃ (328 mg, 2 mmol). The mixture was stirred at 50° C. for 1 hr. The reaction mixture was diluted with H₂O (10 mL) and extracted with EtOAc (20 mL). The combined organic layers were washed with brine (20 mL), dried over Na₂SO₄, filtered and concentrated under reduced pressure. The residue was purified by prep-HPLC (basic condition MeCN/H₂O: 0%-65%) to give 4-((7-(4-(2-(2-Aminopyridin-3-yl)-5-morpholino-3H-imidazo[4,5-b]pyridin-3-yl)benzyl)-7-azaspiro[3.5]nonan-2-yl)amino)-1,3,5-triazine-2-carbonitrile (Example 434, 46 mg, yield: 14%) as a yellow solid. MS: m/z=629.3 [M+H]⁺. ¹H NMR (400 MHz, Dimethylsulfoxide-d₆) δ 9.27-9.08 (m, 1H), 8.74-8.56 (m, 1H), 8.08-7.81 (m, 2H), 7.40 (d, J=7.6 Hz, 2H), 7.36-7.25 (m, 2H), 7.04 (s, 2H), 7.01-6.95 (m, 1H), 6.92-6.86 (m, 1H), 6.29 (dd, J=7.6, 4.8 Hz, 1H), 4.39-4.21 (m, 1H), 3.75-3.65 (m, 4H), 3.50 (s, 2H), 3.45-3.35 (m, 4H), 2.37-2.14 (m, 6H), 1.81-1.72 (m, 2H), 1.64-1.57 (m, 2H), 1.56-1.49 (m, 2H).

### Example 435: 4-((1-(4-(2-(2-Aminopyridin-3-yl)-5-(methylthio)-3H-imidazo[4,5-b]pyridin-3-yl)benzyl)piperidin-4-yl)amino)pyrimidine-2-carbonitrile

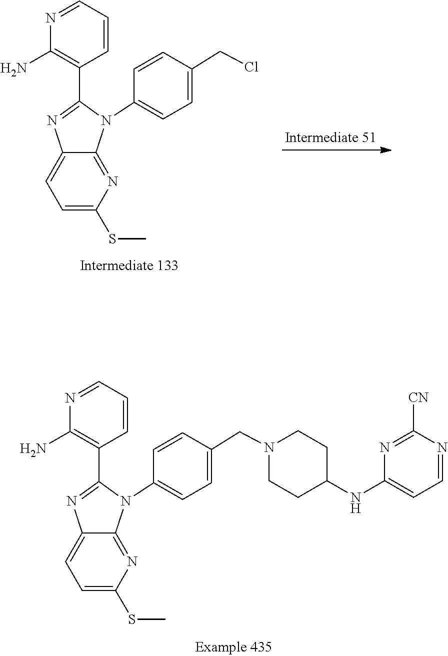 [SMILES: CSc1ccc2nc(-c3cccnc3N)n(-c3ccc(CCl)cc3)c2n1 ; CSc1ccc2nc(-c3cccnc3N)n(-c3ccc(CN4CCC(Nc5ccnc(C#N)n5)CC4)cc3)c2n1]

To a solution of Intermediate 133 (200 mg, 524 μmol) and Intermediate 51 (106 mg, 524 μmol) in DMF (3 mL) were added NaI (15.7 mg, 105 μmol) and K₂CO₃ (217 mg, 1.57 mmol). The mixture was stirred at 25° C. for 16 hr. The residue was diluted with H₂O (10 mL) and extracted with CH₂Cl₂ (20 mL×3). The combined organic layers were washed with brine (20 mL×2), dried over Na₂SO₄, filtered and concentrated under reduced pressure. After purified by prep-HPLC column: Phenomenex C18 75*30 mm*3 μm; mobile phase: [water (0.05% NH₃H₂ 10 mM NH₄HCO₃)-ACN]; gradient: 41%-71% B over 7 min, 4-[[1-[[4-[2-(2-amino-3-pyridyl)-5-methylsulfanyl-imidazo[4,5-b]pyridin-3-yl]phenyl]methyl]-4-piperidyl]amino]pyrimidine-2-carbonitrile (Example 435, 24.4 mg, yield: 8.5%) was obtained as light yellow solid. MS: m/z=571.0 [M+Na]⁺. ¹H NMR (400 MHz, DMSO-d₆) δ 8.11-8.04 (m, 3H), 7.97 (dd, J=4.8, 2.0 Hz, 1H), 7.45 (d, J=8.4 Hz, 2H), 7.38 (d, J=8.4 Hz, 2H), 7.27 (d, J=8.4 Hz, 1H), 7.11 (dd, J=7.6, 1.6 Hz, 1H), 7.0 (br s, 2H), 6.67 (d, J=6.0 Hz, 1H), 6.36 (dd, J=8.0, 5.2 Hz, 1H), 3.91-3.70 (m, 1H), 3.57 (s, 2H), 2.86-2.75 (m, 2H), 2.46 (s, 3H), 2.19-2.07 (m, 2H), 1.94-1.81 (m, 2H), 1.54-1.40 (m, 2H).

### Example 436: 4-((1-(4-(2-(2-Aminopyridin-3-yl)-5-(methylsulfonyl)-3H-imidazo[4,5-b]pyridin-3-yl)benzyl)piperidin-4-yl)amino)pyrimidine-2-carbonitrile

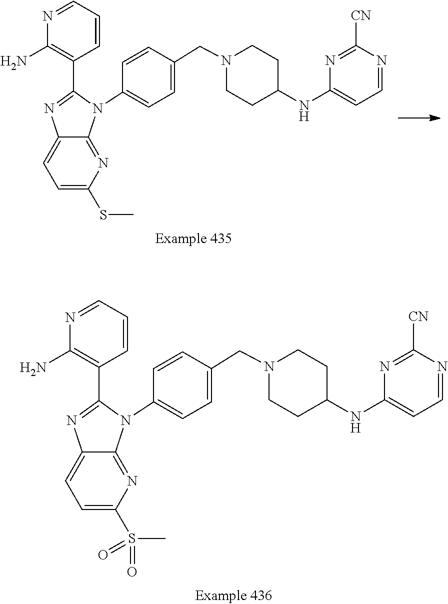 [SMILES: CSc1ccc2nc(-c3cccnc3N)n(-c3ccc(CN4CCC(Nc5ccnc(C#N)n5)CC4)cc3)c2n1 ; CS(=O)(=O)c1ccc2nc(-c3cccnc3N)n(-c3ccc(CN4CCC(Nc5ccnc(C#N)n5)CC4)cc3)c2n1]

To a solution of Example 435 (50 mg, 91.1 μmol) in THF (2 mL) and MeOH (0.5 mL) was added oxone (280 mg, 456 μmol). The mixture was stirred at 25° C. for 16 hr. After purified by prep-HPLC (column: Waters xbridge 150*25 mm 10 μm; mobile phase: [water (NH₄HCO₃)-ACN]; gradient: 18%-48% B over 14 min), 4-((1-(4-(2-(2-Aminopyridin-3-yl)-5-(methylsulfonyl)-3H-imidazo[4,5-b]pyridin-3-yl)benzyl)piperidin-4-yl)amino)pyrimidine-2-carbonitrile (Example 436, 9.1 mg, yield: 16%) was obtained as light yellow solid. MS: m/z=581.3 [M+H]⁺. ¹H NMR (400 MHz, Chloroform-d) δ 8.27 (d, J=8.4 Hz, 1H), 8.18-8.08 (m, 3H), 7.51 (d, J=8.0 Hz, 2H), 7.34 (d, J=8.0 Hz, 2H), 7.09 (dd, J=8.0, 1.6 Hz, 1H), 6.67 (br s, 2H), 6.44 (d, J=6.0 Hz, 1H), 6.36 (dd, 1=7.6, 4.8 Hz, 1H), 3.65 (s, 2H), 3.19 (s, 3H), 2.99-2.89 (m, 2H), 2.33-2.25 (m, 2H), 2.10-2.04 (m, 2H), 1.76-1.66 (m, 3H).

### Example 437: 4-((1-(4-(2-(2-Aminopyridin-3-yl)-5-(4-methoxypyridin-2-yl)-3H-imidazo[4,5-b]pyridin-3-yl)benzyl)piperidin-4-yl)amino)pyrimidine-2-carbonitrile

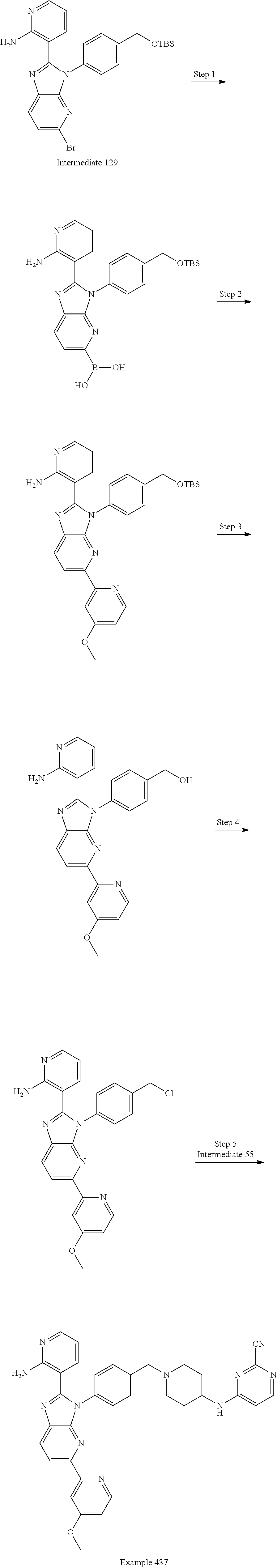 [SMILES: CC(C)(C)[Si](C)(C)OCc1ccc(-n2c(-c3cccnc3N)nc3ccc(Br)nc32)cc1 ; CC(C)(C)[Si](C)(C)OCc1ccc(-n2c(-c3cccnc3N)nc3ccc(B(O)O)nc32)cc1 ; COc1ccnc(-c2ccc3nc(-c4cccnc4N)n(-c4ccc(CO[Si](C)(C)C(C)(C)C)cc4)c3n2)c1 ; COc1ccnc(-c2ccc3nc(-c4cccnc4N)n(-c4ccc(CO)cc4)c3n2)c1 ; COc1ccnc(-c2ccc3nc(-c4cccnc4N)n(-c4ccc(CCl)cc4)c3n2)c1 ; COc1ccnc(-c2ccc3nc(-c4cccnc4N)n(-c4ccc(CN5CCC(Nc6ccnc(C#N)n6)CC5)cc4)c3n2)c1]

### Step 1: (2-(2-Aminopyridin-3-yl)-3-(4-(((tert-butyldimethylsilyl)oxy)methyl)phenyl)-3H-imidazo[4,5-b]pyridin-5-yl)boronic acid

A mixture of Intermediate 129 (200 mg, 392 μmol), 4,4,4′,4′,5,5,5′,5′-octamethyl-2,2′-bi(1,3,2-dioxaborolane) (199 mg, 784 μmol), KOAc (115 mg, 1.18 mmol) and Pd(dppf)Cl₂ (28.7 mg, 39.2 μmol) in 1,4-dioxane (2 mL) was degassed and purged with N₂ three times. The mixture was stirred at 80° C. for 2 hr under N₂ atmosphere. (2-(2-Aminopyridin-3-yl)-3-(4-(((tert-butyldimethylsilyl)oxy)methyl)phenyl)-3H-imidazo[4,5-b]pyridin-5-yl)boronic acid was obtained as a black liquid, which was used in the next step directly without work-up and purification. MS: m/z=476.1 [M+H]⁺.

### Step 2: 3-(3-(4-(((Tert-butyldimethylsilyl)oxy)methyl)phenyl)-5-(4-methoxypyridin-2-yl)-3H-imidazo[4,5-b]pyridin-2-yl)pyridin-2-amine

A mixture of (2-(2-aminopyridin-3-yl)-3-(4-(((tert-butyldimethylsilyl)oxy)methyl)phenyl)-3H-imidazo[4,5-b]pyridin-5-yl)boronic acid (460 mg, 968 μmol), 2-bromo-4-methoxypyridine (182 mg, 968 μmol), Pd(dppf)Cl₂ (70.8 mg, 96.8 μmol) and Cs₂CO₃ (946 mg, 2.90 mmol) in 1,4-dioxane (5 mL) and H₂O (1 mL) was degassed and purged with N₂ three times. The mixture was stirred at 80° C. for 16 hr under N₂ atmosphere. The reaction mixture was quenched with H₂O (10 mL), diluted with EtOAc (10 mL) and extracted with EtOAc (15 mL×2). The combined organic layers were washed with brine (30 mL), dried over Na₂SO₄, filtered, and concentrated under reduced pressure. After purified by silica gel flash chromatography (Eluent of 10%˜40% EtOAc in petroleum ether), 3-(3-(4-(((tert-butyldimethylsilyl)oxy)methyl)phenyl)-5-(4-methoxypyridin-2-yl)-3H-imidazo[4,5-b]pyridin-2-yl)pyridin-2-amine (250 mg, yield: 56% for two steps) was obtained as a black solid. MS: m/z=539.2 [M+H]⁺. ¹H NMR (400 MHz, Chloroform-d) 68.54-8.46 (m, 2H), 8.17 (d, J=8.4 Hz, 1H), 8.06 (dd, J=4.8, 1.6 Hz, 1H), 7.88 (d, J=2.4 Hz, 1H), 7.52-7.48 (m, 2H), 7.43-7.38 (m, 2H), 7.14 (dd, J=7.6, 1.2 Hz, 1H), 6.80 (dd, J=5.6, 2.4 Hz, 1H), 6.71 (br s, 2H), 6.38 (dd, J=7.6, 4.8 Hz, 1H), 4.86 (s, 2H), 3.87 (s, 3H), 0.98 (s, 9H), 0.15 (s, 6H).

### Step 3: (4-(2-(2-Aminopyridin-3-yl)-5-(4-methoxypyridin-2-yl)-3H-imidazo[4,5-b]pyridin-3-yl)phenyl)methanol

To a solution of 3-(3-(4-(((tert-butyldimethylsilyl)oxy)methyl)phenyl)-5-(4-methoxypyridin-2-yl)-3H-imidazo[4,5-b]pyridin-2-yl)pyridin-2-amine (250 mg, 464 μmol) in THE (3 mL) was added TBAF (364 mg, 1.39 mmol, 1 M in THF). The mixture was stirred at 25° C. for 0.5 hr. The reaction mixture was quenched with H₂O (5 mL) at 25° C., diluted with CH₂Cl₂ (10 mL) and extracted with CH₂Cl₂ (15 mL×3). The combined organic layers were washed with brine (50 mL×2), dried over Na₂SO₄, filtered and concentrated under reduced pressure. The mixture was triturated with EtOAc:petroleum ether=1:10 at 25° C. for 10 min. (4-(2-(2-Aminopyridin-3-yl)-5-(4-methoxypyridin-2-yl)-3H-imidazo[4,5-b]pyridin-3-yl)phenyl)methanol (160 mg, yield: 81%) was obtained as a black solid. MS: m/z=425.5 [M+H]⁺. ¹H NMR (400 MHz, Dimethylsulfoxide-d₆) δ 8.52 (d, J=5.6 Hz, 1H), 8.44 (d, J=8.4 Hz, 1H), 8.29 (d, J=8.4 Hz, 1H), 8.01 (dd, J=4.8, 2.0 Hz, 1H), 7.70 (d, J=2.4 Hz, 1H), 7.54-7.45 (m, 4H), 7.25 (dd, J=7.6, 2.0 Hz, 1H), 7.03 (dd, J=5.6, 2.4 Hz, 1H), 6.97 (br s, 2H), 6.43 (dd, J=7.6, 4.8 Hz, 1H), 5.36 (t, J=5.6 Hz, 1H), 4.61 (d, J=5.6 Hz, 2H), 3.85 (s, 3H).

### Step 4: 3-(3-(4-(Chloromethyl)phenyl)-5-(4-methoxypyridin-2-yl)-3H-imidazo[4,5-b]pyridin-2-yl)pyridin-2-amine

To a solution of (4-(2-(2-aminopyridin-3-yl)-5-(4-methoxypyridin-2-yl)-3H-imidazo[4,5-b]pyridin-3-yl)phenyl)methanol (160 mg, 377 μmol) in CH₂Cl₂ (5 mL) was added SOCl₂ (135 mg, 1.13 mmol). The mixture was stirred at 25° C. for 0.4 hr. The reaction was concentrated under reduced pressure to give 3-(3-(4-(chloromethyl)phenyl)-5-(4-methoxypyridin-2-yl)-3H-imidazo[4,5-b]pyridin-2-yl)pyridin-2-amine (180 mg, HCl salt) as a yellow solid. MS: m/z=443.0 [M+H]⁺. ¹H NMR (400 MHz, Methanol-d₄) δ 8.62 (d, J=6.8 Hz, 1H), 8.58-8.55 (m, 1H), 8.52-8.48 (m, 1H), 8.24 (d, J=2.4 Hz, 1H), 8.07-8.04 (m, 1H), 7.85 (d, J=6.4 Hz, 1H), 7.73 (d, J=8.0 Hz, 2H), 7.63 (d, J=8.4 Hz, 2H), 7.55 (dd, J=7.2, 2.4 Hz, 1H), 6.91-6.84 (m, 1H), 4.80 (s, 2H), 4.26 (s, 3H).

### Step 5: 4-((1-(4-(2-(2-Aminopyridin-3-yl)-5-(4-methoxypyridin-2-yl)-3H-imidazo[4,5-b]pyridin-3-yl)benzyl)piperidin-4-yl)amino)pyrimidine-2-carbonitrile

To a solution of 3-(3-(4-(chloromethyl)phenyl)-5-(4-methoxypyridin-2-yl)-3H-imidazo[4,5-b]pyridin-2-yl)pyridin-2-amine (180 mg, 376 μmol, HCl salt) and Intermediate 55 (119 mg, 376 μmol, TFA salt) in DMF (4 mL) were added K₂CO₃ (259 mg, 1.88 mmol) and NaI (5.63 mg, 37.6 μmol). The mixture was stirred at 50° C. for 1 hr. The reaction mixture was quenched with H₂O (5 mL) at 25° C., diluted with CH₂Cl₂ (10 mL) and extracted with CH₂Cl₂ (15 mL×2). The combined organic layers were washed with brine (50 mL×3), dried over Na₂SO₄, filtered, and concentrated under reduced pressure. After purified by silica gel flash chromatography (Eluent of 1%˜7% MeOH in CH₂Cl₂), 4-((1-(4-(2-(2-aminopyridin-3-yl)-5-(4-methoxypyridin-2-yl)-3H-imidazo[4,5-b]pyridin-3-yl)benzyl)piperidin-4-yl)amino)pyrimidine-2-carbonitrile (Example 437, 93.9 mg, yield: 40% for two steps) was obtained as a yellow solid. MS: m/z=610.2 [M+H]⁺. ¹H NMR (400 MHz, Dimethylsulfoxide-d₆) δ 8.51 (d, J=5.6 Hz, 1H), 8.45 (d, J=8.4 Hz, 1H), 8.30 (d, J=8.4 Hz, 1H), 8.14-7.98 (m, 3H), 7.74-7.71 (m, 1H), 7.54-7.45 (m, 4H), 7.22 (d, J=7.2 Hz, 1H), 7.06-7.00 (m, 3H), 6.68 (d, J=5.6 Hz, 1H), 6.41 (dd, J=7.6, 4.8 Hz, 1H), 3.83 (s, 3H), 3.82-3.75 (m, 1H), 3.61 (s, 2H), 2.87-2.80 (m, 2H), 2.20-2.11 (m, 2H), 1.95-1.85 (m, 2H), 1.57-1.46 (m, 2H).

### Example 438: 4-((1-(4-(2-(2-Aminopyridin-3-yl)-5-(1-methyl-1H-1,2,3-triazol-4-yl)-3H-imidazo[4,5-b]pyridin-3-yl)benzyl)piperidin-4-yl)amino)pyrimidine-2-carbonitrile

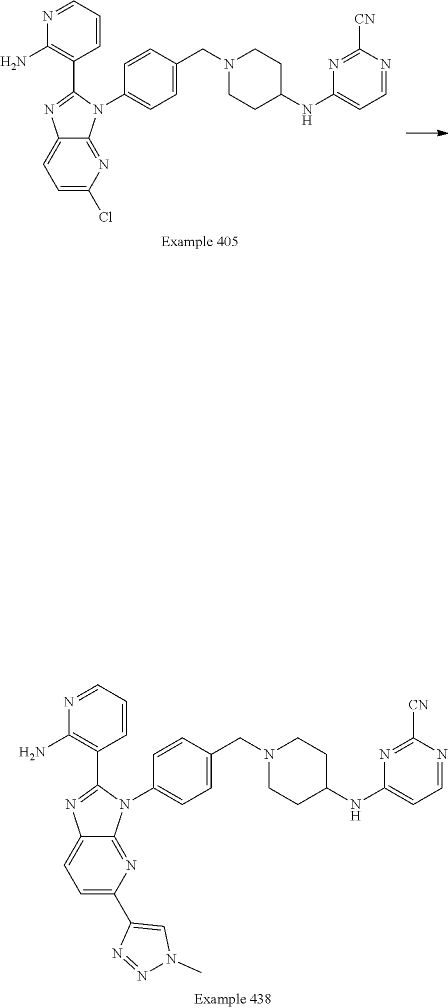 [SMILES: N#Cc1nccc(NC2CCN(Cc3ccc(-n4c(-c5cccnc5N)nc5ccc(Cl)nc54)cc3)CC2)n1 ; Cn1cc(-c2ccc3nc(-c4cccnc4N)n(-c4ccc(CN5CCC(Nc6ccnc(C#N)n6)CC5)cc4)c3n2)nn1]

To a solution of 4-bromo-1-methyl-1H-1,2,3-triazole (400 mg, 2.5 mmol) and 4-(4,4,5,5-tetramethyl-1,3,2-dioxaborolan-2-yl)pyridine-2-carbonitrile (272 mg, 1.18 mmol) in 1,4-dioxane (4 mL) were added Pd(dppf)Cl₂ (101 mg, 123 μmol) and KOAc (1.05 g, 3.22 mmol) at 25° C. The mixture was degassed and purged with N₂ three times and stirred at 100° C. for 12 hr. Then 4-((1-(4-(2-(2-aminopyridin-3-yl)-5-chloro-3H-imidazo[4,5-b]pyridin-3-yl)benzyl)piperidin-4-yl)amino)pyrimidine-2-carbonitrile (200 mg, 372 mmol), Cs₂CO₃ (360 mg, 1.1 mmol) and Pd(dppf)Cl₂ (30 mg, 37 μmol) at 25° C. were added to the above mixture. The mixture was degassed and purged with N₂ three times and stirred at 100° C. for 12 hr. The mixture was quenched with H₂O (50 mL) and extracted with EtOAc (50 mL×3). The combined organic layers were washed with brine (50 mL×3), dried over Na₂SO₄, filtered and concentrated. After purified by silica gel flash chromatography (Eluent of 0˜7% MeOH in CH₂Cl₂), 4-((1-(4-(2-(2-aminopyridin-3-yl)-5-(1-methyl-1H-1,2,3-triazol-4-yl)-3H-imidazo[4,5-b]pyridin-3-yl)benzyl)piperidin-4-yl)amino)pyrimidine-2-carbonitrile (Example 438, 18.7 mg, yield: 8% for two steps) was obtained as an off white solid. MS: m/z=584.2 [M+1]⁺. ¹H NMR (400 MHz, Methanol-d₄) δ 8.15 (s, 1H), 8.09 (d, J=8.4 Hz, 1H), 7.98 (d, J=8.4 Hz, 1H), 7.93-7.83 (m, 2H), 7.42 (d, J=8.4 Hz, 2H), 7.31 (d, J=8.4 Hz, 2H), 7.16 (dd, J=7.6, 1.6 Hz, 1H), 6.50 (d, J=4.4 Hz, 1H), 6.35 (dd, J=7.6, 5.2 Hz, 1H), 4.01 (s, 3H), 3.93-3.79 (m, 1H), 3.57 (s, 2H), 2.92-2.81 (m, 2H), 2.25-2.13 (m, 2H), 1.93-1.89 (m, 2H), 1.58-1.45 (m, 2H).

### Example 439: 4-((1-(4-(2-(2-Aminopyridin-3-yl)-5-(4-fluoro-3-methoxyphenyl)-3H-imidazo[4,5-b]pyridin-3-yl)benzyl)piperidin-4-yl)amino)pyrimidine-2-carbonitrile

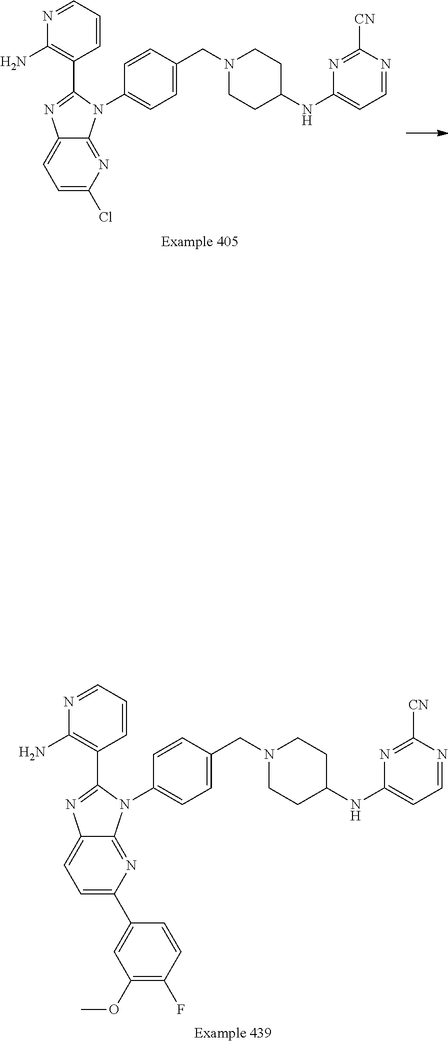 [SMILES: N#Cc1nccc(NC2CCN(Cc3ccc(-n4c(-c5cccnc5N)nc5ccc(Cl)nc54)cc3)CC2)n1 ; COc1cc(-c2ccc3nc(-c4cccnc4N)n(-c4ccc(CN5CCC(Nc6ccnc(C#N)n6)CC5)cc4)c3n2)ccc1F]

To a solution of Example 405 (200 mg, 372 μmol) and (4-fluoro-3-methoxy-phenyl)boronic acid (69.6 mg, 410 μmol) in 1,4-dioxane (10 mL) and H₂O (2 mL) were added Cs₂CO₃ (364 mg, 1.12 mmol) and Pd(PPh₃)₄ (43.1 mg, 37.2 μmol) at 25° C. The mixture was degassed and purged with N₂ three times and stirred at 100° C. for 2 hr. The reaction mixture was quenched with water (20 mL) and extracted with EtOAc (20 mL×3). The combined organic layers were washed with brine (20 mL), dried over Na₂SO₄, filtered and concentrated. After purified by prep-HPLC (column: C18 150×30 mm; mobile phase: [water (NH₃H₂O+NH₄HCO₃)-ACN]; gradient: 5%-84% B over 7 min), 4-((1-(4-(2-(2-aminopyridin-3-yl)-5-(4-fluoro-3-methoxyphenyl)-3H-imidazo[4,5-b]pyridin-3-yl)benzyl)piperidin-4-yl)amino)pyrimidine-2-carbonitrile (Example 439, 130 mg, yield: 56%) was obtained as a light yellow solid. MS: m/z=627.3 [M+H]⁺. ¹H NMR (400 MHz, Dimethylsulfoxide-d₆) δ 8.27 (d, J=8.4 Hz, 1H), 8.14-7.96 (m, 4H), 7.81 (dd, J=8.4, 1.6 Hz, 1H), 7.68-7.59 (m, 1H), 7.52-7.42 (m, 4H), 7.29 (dd, J=11.2, 8.8 Hz, 1H), 7.18 (dd, J=7.6, 1.6 Hz, 1H), 7.06 (s, 2H), 6.68 (d, J=6.0 Hz, 1H), 6.39 (dd, J=7.6, 4.8 Hz, 1H), 3.89 (s, 3H), 3.86-3.74 (m, 1H), 3.59 (s, 2H), 2.88-2.77 (m, 2H), 2.21-2.09 (m, 2H), 1.96-1.81 (m, 2H), 1.58-1.40 (m, 2H). ¹⁹F NMR (400 MHz, Dimethylsulfoxide-d₆) δ −135.378.

### Example 440: N-(3-(2-(2-Aminopyridin-3-yl)-3-(4-((4-((2-cyanopyrimidin-4-yl)amino)piperidin-1-yl)methyl)phenyl)-3H-imidazo[4,5-b]pyridin-5-yl)phenyl)acetamide

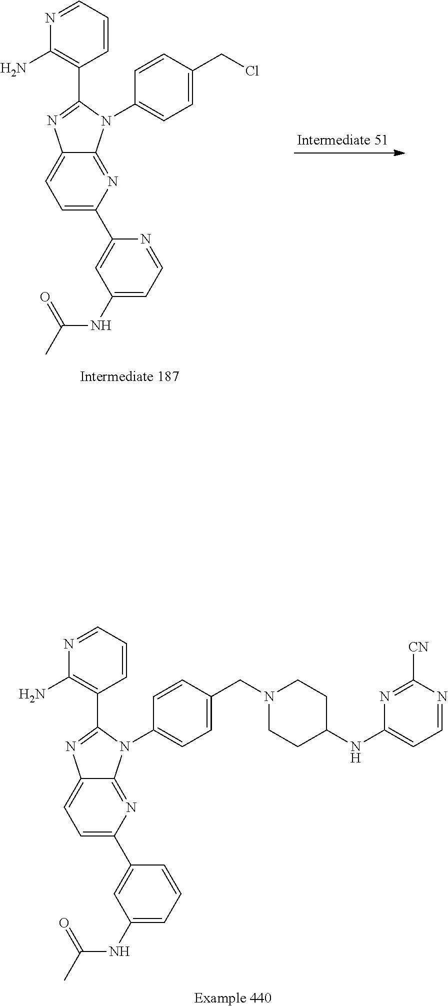 [SMILES: CC(=O)Nc1ccnc(-c2ccc3nc(-c4cccnc4N)n(-c4ccc(CCl)cc4)c3n2)c1 ; CC(=O)Nc1cccc(-c2ccc3nc(-c4cccnc4N)n(-c4ccc(CN5CCC(Nc6ccnc(C#N)n6)CC5)cc4)c3n2)c1]

To a solution of Intermediate 187 (220 mg, 469 μmol) and Intermediate 51 (200 mg, 630 μmol) in DMF (10 mL) were added K₂CO₃ (324 mg, 2.35 mmol) and NaI (21.1 mg, 140 μmol). The mixture was stirred at 50° C. for 1 hr. The reaction mixture was diluted with H₂O (50 mL) and extracted with EtOAc (50 mL×2). The combined organic layers were washed with H₂O (50 mL×6), dried over Na₂SO₄, filtered, and concentrated under reduced pressure. After purified by prep-HPLC (column: Daisogel SP ODS RPS 150*25 mm*5 μm; mobile phase: [water (NH₄HCO₃)-ACN]; gradient: 32%-62% B over 12 min), N-(3-(2-(2-aminopyridin-3-yl)-3-(4-((4-((2-cyanopyrimidin-4-yl)amino)piperidin-1-yl)methyl)phenyl)-3H-imidazo[4,5-b]pyridin-5-yl)phenyl)acetamide (Example 440, 101.5 mg, yield: 32%) was obtained as a light-yellow powder. MS: m/z=636.2 [M+H]⁺. ¹H NMR (400 MHz, Dimethylsulfoxide-d₆) δ 10.05 (s, 1H), 8.28 (d, J=8.4 Hz, 1H), 8.15 (s, 1H), 8.13-8.01 (m, 2H), 7.99 (dd, J=4.8, 1.6 Hz, 1H), 7.86 (d, J=8.4 Hz, 1H), 7.69-7.63 (m, 2H), 7.50-7.43 (m, 4H), 7.38 (t, J=8.0 Hz, 1H), 7.14 (dd, J=7.6, 1.6 Hz, 1H), 7.01 (br s, 2H), 6.67 (d, J=6.0 Hz, 1H), 6.37 (dd, J=7.6, 4.8 Hz, 1H), 3.95-3.68 (m, 1H), 3.59 (s, 2H), 2.88-2.79 (m, 2H), 2.19-2.11 (m, 2H), 2.03 (s, 3H), 1.95-1.75 (m, 2H), 1.56-1.43 (m, 2H).

### Example 441: 4-((1-(4-(2-(2-Aminopyridin-3-yl)-5-(4-fluorophenyl)-3H-imidazo[4,5-b]pyridin-3-yl)benzyl)piperidin-4-yl)(methyl-d3)amino)pyrimidine-2-carbonitrile

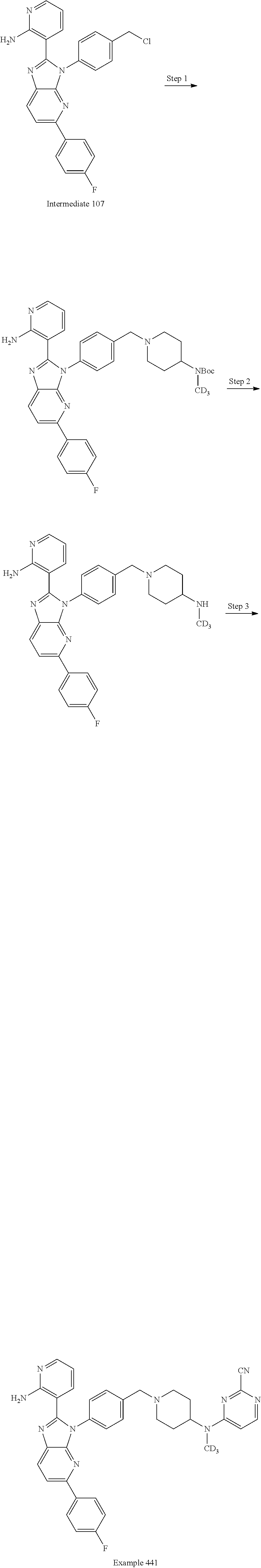 [SMILES: Nc1ncccc1-c1nc2ccc(-c3ccc(F)cc3)nc2n1-c1ccc(CCl)cc1 ; [2H]C([2H])([2H])N(C(=O)OC(C)(C)C)C1CCN(Cc2ccc(-n3c(-c4cccnc4N)nc4ccc(-c5ccc(F)cc5)nc43)cc2)CC1 ; [2H]C([2H])([2H])NC1CCN(Cc2ccc(-n3c(-c4cccnc4N)nc4ccc(-c5ccc(F)cc5)nc43)cc2)CC1 ; [2H]C([2H])([2H])N(c1ccnc(C#N)n1)C1CCN(Cc2ccc(-n3c(-c4cccnc4N)nc4ccc(-c5ccc(F)cc5)nc43)cc2)CC1]

### Step 1: Tert-butyl (1-(4-(2-(2-aminopyridin-3-yl)-5-(4-fluorophenyl)-3H-imidazo[4,5-b]pyridin-3-yl)benzyl)piperidin-4-yl)(methyl-d3)carbamate

To a solution of Intermediate 107 (100 mg, 232 μmol) in DMF (3 mL) were added tert-butyl (methyl-d3)(piperidin-4-yl)carbamate (refer to Intermediate 41 for detail procedures, 60 mg, 279 μmol), NaI (35 mg, 233 μmol) and K₂CO₃ (129 mg, 930 μmol) at 25° C. The mixture was stirred at 50° C. for 1 hr. The reaction mixture was poured into H₂O (50 mL). The mixture was extracted with CH₂Cl₂ (20 mL×2). The combined organic layers were washed with brine (40 mL), dried over anhydrous Na₂SO₄, filtered, and concentrated to dryness. After purified by silica gel flash chromatography (MeOH in CH₂Cl₂=0% to 3%), tert-butyl (1-(4-(2-(2-aminopyridin-3-yl)-5-(4-fluorophenyl)-3H-imidazo[4,5-b]pyridin-3-yl)benzyl)piperidin-4-yl)(methyl-d3)carbamate (130 mg, yield: 71%) was obtained as a yellow solid. MS: m/z=611.1 [M+H]⁺. ¹H NMR (400 MHz, Dimethylsulfoxide-d₆) δ 8.27 (d, J=8.4 Hz, 1H), 8.12-8.05 (m, 2H), 8.01-7.96 (m, 2H), 7.50-7.41 (m, 4H), 7.34-7.27 (m, 2H), 7.15 (dd, J=8.0, 2.0 Hz, 1H), 7.02 (s, 2H), 6.37 (dd, J=7.6, 4.8 Hz, 1H), 3.88-3.67 (m, 1H), 3.57 (s, 2H), 2.96-2.86 (m, 2H), 2.08-1.97 (m, 2H), 1.74-1.63 (m, 2H), 1.56-1.47 (m, 2H), 1.40 (s, 9H). ¹⁹F NMR (400 MHz, Dimethylsulfoxide-d₆) δ −113.59 (s, 1F).

### Step 2: 3-(5-(4-Fluorophenyl)-3-(4-((4-((methyl-d3)amino)piperidin-1-yl)methyl)phenyl)-3H-imidazo[4,5-b]pyridin-2-yl)pyridin-2-amine

To a solution of tert-butyl (1-(4-(2-(2-aminopyridin-3-yl)-5-(4-fluorophenyl)-3H-imidazo[4,5-b]pyridin-3-yl)benzyl)piperidin-4-yl)(methyl-d3)carbamate (130 mg, 213 μmol) in CH₂Cl₂ (5 mL) was added TFA (121 mg, 1.1 mmol) at 25° C. The reaction mixture was stirred at 25° C. for 1 hr. The reaction mixture was concentrated under reduced pressure. 3-(5-(4-Fluorophenyl)-3-(4-((4-((methyl-d3)amino)piperi din-1-yl)methyl)phenyl)-3H-imidazo[4,5-b]pyridin-2-yl)pyridin-2-amine (100 mg) was used into the next step without further purification. MS: m/z=511.2 [M+H]⁺.

### Step 3: 4-((1-(4-(2-(2-Aminopyridin-3-yl)-5-(4-fluorophenyl)-3H-imidazo[4,5-b]pyridin-3-yl)benzyl)piperidin-4-yl)(methyl-d3)amino)pyrimidine-2-carbonitrile

To a solution of 3-(5-(4-fluorophenyl)-3-(4-((4-((methyl-d3)amino)piperidin-1-yl)methyl)phenyl)-3H-imidazo[4,5-b]pyridin-2-yl)pyridin-2-amine (100 mg, 196 μmol) and 4-chloropyrimidine-2-carbonitrile (33 mg, 235 μmol) in NMP (3 mL) was added DIEA (76 mg, 588 μmol) at 25° C. The mixture was stirred at 130° C. for 1 hr. The reaction mixture was poured into H₂O (50 mL) and extracted with CH₂Cl₂ (20 mL×2). The combined organic layers were washed with brine (40 mL), dried over anhydrous Na₂SO₄, filtered and concentrated. After purified by silica gel flash chromatography (Eluent of 0˜5% MeOH in CH₂Cl₂), 4-((1-(4-(2-(2-aminopyridin-3-yl)-5-(4-fluorophenyl)-3H-imidazo[4,5-b]pyridin-3-yl)benzyl)piperidin-4-yl)(methyl-d3)amino)pyrimidine-2-carbonitrile (Example 441, 21.6 mg, yield: 17% for two steps) was obtained as a yellow solid. MS: m/z=614.7 [M+H]⁺. ¹H NMR (400 MHz, Dimethylsulfoxide-d₆) δ 8.27 (d, J=8.4 Hz, 1H), 8.24 (d, J=6.4 Hz, 1H), 8.10-8.06 (m, 2H), 8.01-7.97 (m, 2H), 7.52-7.43 (m, 5H), 7.30 (t, J=8.8 Hz, 2H), 7.15 (dd, J=7.6, 1.6 Hz, 1H), 7.03 (s, 2H), 6.38 (dd, J=7.6, 4.8 Hz, 1H), 3.62 (s, 2H), 3.29-3.28 (m, 1H), 2.99-2.91 (m, 2H), 2.21-2.14 (m, 2H), 1.89-1.79 (m, 2H), 1.63-1.56 (m, 2H). ¹⁹F NMR (400 MHz, Dimethylsulfoxide-d₆) δ −113.58.

### Example 442: 4-((1-((4-(2-(2-Aminopyridin-3-yl)-5-(4-fluorophenyl)-3H-imidazo[4,5-b]pyridin-3-yl)phenyl)methyl-d2)piperidin-4-yl)(methyl-d3)amino)pyrimidine-2-carbonitrile

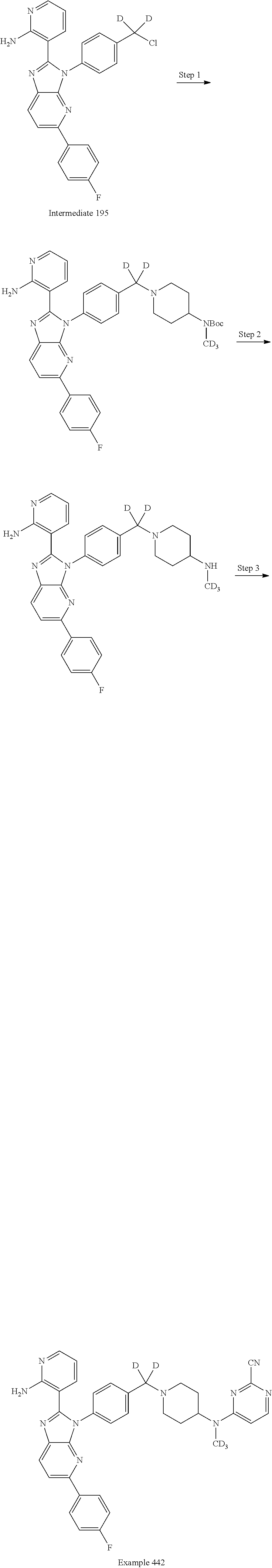 [SMILES: [2H]C([2H])(Cl)c1ccc(-n2c(-c3cccnc3N)nc3ccc(-c4ccc(F)cc4)nc32)cc1 ; [2H]C([2H])([2H])N(C(=O)OC(C)(C)C)C1CCN(C([2H])([2H])c2ccc(-n3c(-c4cccnc4N)nc4ccc(-c5ccc(F)cc5)nc43)cc2)CC1 ; [2H]C([2H])([2H])NC1CCN(C([2H])([2H])c2ccc(-n3c(-c4cccnc4N)nc4ccc(-c5ccc(F)cc5)nc43)cc2)CC1 ; [2H]C([2H])([2H])N(c1ccnc(C#N)n1)C1CCN(C([2H])([2H])c2ccc(-n3c(-c4cccnc4N)nc4ccc(-c5ccc(F)cc5)nc43)cc2)CC1]

### Step 1: Tert-butyl (1-((4-(2-(2-aminopyridin-3-yl)-5-(4-fluorophenyl)-3H-imidazo[4,5-b]pyridin-3-yl)phenyl)methyl-d2)piperidin-4-yl)(methyl-d3)carbamate

To a solution of Intermediate 195 (100 mg, 232 μmol) in DMF (3 mL) were added tert-butyl (methyl-d3)(piperidin-4-yl)carbamate (refer to Intermediate 41 for detail procedures, 60 mg, 279 μmol), NaI (35 mg, 232 μmol) and K₂CO₃ (128 mg, 926 μmol) at 25° C., the mixture was stirred at 50° C. for 1 hr. The reaction mixture was poured into H₂O (50 mL), extracted with CH₂Cl₂ (20 mL×2). The combined organic layers were washed with brine (40 mL), dried over anhydrous Na₂SO₄, filtered and concentrated. After purified by silica gel flash chromatography (Eluent of 0˜3% MeOH in CH₂Cl₂), tert-butyl (1-((4-(2-(2-aminopyridin-3-yl)-5-(4-fluorophenyl)-3H-imidazo[4,5-b]pyridin-3-yl)phenyl)methyl-d2)piperidin-4-yl)(methyl-d3)carbamate (130 mg, yield: 70%) was obtained as a yellow solid. MS: m/z=613.2 [M+H]⁺. ¹H NMR (400 MHz, Dimethylsulfoxide-d₆) δ 8.27 (d, J=8.4 Hz, 1H), 8.11-8.04 (m, 2H), 8.01-7.94 (m, 2H), 7.51-7.40 (m, 4H), 7.30 (t, J=8.8 Hz, 2H), 7.14 (dd, J=7.6, 2.0 Hz, 1H), 7.03 (s, 2H), 6.37 (dd, J=8.0, 4.8 Hz, 1H), 3.81-3.79 (m, 1H), 2.95-2.85 (m, 2H), 2.07-1.99 (m, 2H), 1.76-1.62 (m, 2H), 1.56-1.47 (m, 2H), 1.39 (s, 9H). ¹⁹F NMR (400 MHz, Dimethylsulfoxide-d₆) δ −113.59.

### Step 2: 3-(5-(4-Fluorophenyl)-3-(4-((4-((methyl-d3)amino)piperidin-1-yl)methyl-d₂)phenyl)-3H-imidazo[4,5-b]pyridin-2-yl)pyridin-2-amine

To a solution of tert-butyl (1-((4-(2-(2-aminopyridin-3-yl)-5-(4-fluorophenyl)-3H-imidazo[4,5-b]pyridin-3-yl)phenyl)methyl-d2)piperidin-4-yl)(methyl-d3)carbamate (130 mg, 212 μmol) in CH₂Cl₂ (5 mL) was added TFA (121 mg, 1.1 mmol) at 25° C. The reaction mixture was stirred at 25° C. for 1 hr. The reaction mixture was concentrate under reduced pressure. The crude product 3-(5-(4-fluorophenyl)-3-(4-((4-((methyl-d3)amino)piperidin-1-yl)methyl-d2)phenyl)-3H-imidazo[4,5-b]pyridin-2-yl)pyridin-2-amine (105 mg) was used into the next step without further purification. MS: m/z=513.1 [M+H]⁺.

### Step 3: 4-((1-((4-(2-(2-Aminopyridin-3-yl)-5-(4-fluorophenyl)-3H-imidazo[4,5-b]pyridin-3-yl)phenyl)methyl-d2)piperidin-4-yl)(methyl-d3)amino)pyrimidine-2-carbonitrile

To a solution of 3-(5-(4-fluorophenyl)-3-(4-((4-((methyl-d3)amino)piperidin-1-yl)methyl-d₂)phenyl)-3H-imidazo[4,5-b]pyridin-2-yl)pyridin-2-amine (100 mg, 195 μmol) and 4-chloropyrimidine-2-carbonitrile (33 mg, 235 μmol) in NMP (3 mL) was added DIEA (76 mg, 588 μmol) at 25° C. The mixture was stirred at 130° C. for 1 hr. The reaction mixture was poured into H₂O (50 mL), extracted with CH₂Cl₂ (20 mL×2). The combined organic layers were washed with brine (40 mL), dried over anhydrous Na₂SO₄, filtered and concentrated. After purified by silica gel flash chromatography (Eluent of 0˜5% MeOH in CH₂Cl₂), 4-((1-((4-(2-(2-aminopyridin-3-yl)-5-(4-fluorophenyl)-3H-imidazo[4,5-b]pyridin-3-yl)phenyl)methyl-d2)piperidin-4-yl)(methyl-d3)amino)pyrimidine-2-carbonitrile (Example 442, 20.5 mg, yield: 17% for two steps) was obtained as a yellow solid. MS: m/z=616.5 [M+H]⁺. ¹H NMR (400 MHz, Dimethylsulfoxide-d₆) δ 8.27 (d, J=8.4 Hz, 1H), 8.24 (d, J=6.4 Hz, 1H), 8.08 (dd, J=8.8, 5.6 Hz, 2H), 8.01-7.97 (m, 2H), 7.55-7.42 (m, 5H), 7.30 (t, J=8.8 Hz, 2H), 7.15 (dd, J=7.6, 1.6 Hz, 1H), 7.07-6.92 (m, 2H), 6.38 (dd, J=7.6, 4.8 Hz, 1H), 3.29-3.28 (m, 1H), 2.99-2.92 (m, 2H), 2.22-2.13 (m, 2H), 1.88-1.79 (m, 2H), 1.65-1.55 (m, 2H). ¹⁹F NMR (400 MHz, Dimethylsulfoxide-d₆) δ −113.58.

### Example 443: 4-((1-(4-(2-(2-Aminopyridin-3-yl)-5-(4-fluorophenyl)-3H-imidazo[4,5-b]pyridin-3-yl)benzyl)azetidin-3-yl)amino)pyrimidine-2-carbonitrile

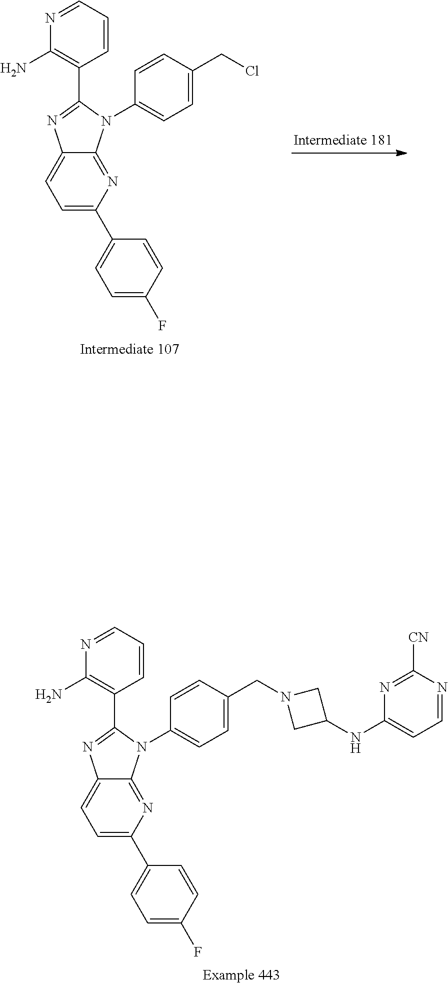 [SMILES: Nc1ncccc1-c1nc2ccc(-c3ccc(F)cc3)nc2n1-c1ccc(CCl)cc1 ; N#Cc1nccc(NC2CN(Cc3ccc(-n4c(-c5cccnc5N)nc5ccc(-c6ccc(F)cc6)nc54)cc3)C2)n1]

To a solution of Intermediate 107 3 (200 mg, 465 μmol), Intermediate 181 (148 mg, 512 μmol, TFA salt) in DMF (2 mL) were added NaI (21 mg, 140 μmol) and K₂CO₃ (322 mg, 2.33 mmol). The mixture was stirred at 50° C. for 1 hr. The reaction mixture was poured into H₂O (10 mL) and extracted with EtOAc (15 mL×3). The combined organic layers were dried over anhydrous Na₂SO₄, filtered and concentrated under reduced pressure. After purified by prep-HPLC (column: Waters Xbridge 150×25 mm×5 μm; mobile phase: [water (NH₃H₂O+NH₄HCO₃)-ACN]; gradient: 43%-73% B over 11 min), 4-((1-(4-(2-(2-aminopyridin-3-yl)-5-(4-fluorophenyl)-3H-imidazo[4,5-b]pyridin-3-yl)benzyl)azetidin-3-yl)amino)pyrimidine-2-carbonitrile (Example 443, 93.2 mg, yield: 35%) was obtained as a yellow lyophilized powder. MS: m/z=569.3 [M+H]⁺. ¹H NMR (400 MHz, Dimethylsulfoxide-d₆) δ 8.64 (d, J=6.8 Hz, 1H), 8.27 (d, J=8.4 Hz, 1H), 8.15 (d, J=6.0 Hz, 1H), 8.11-8.05 (m, 2H), 8.02-7.97 (m, 2H), 7.49-7.42 (m, 4H), 7.30 (t, J=8.8 Hz, 2H), 7.16 (dd, J=7.6, 1.6 Hz, 1H), 7.01 (br s, 2H), 6.73 (d, J=6.0 Hz, 1H), 6.40 (dd, J=7.6, 4.8 Hz, 1H), 4.56-4.38 (m, 1H), 3.71 (s, 2H), 3.68-3.61 (m, 2H), 3.04 (t, J=6.4 Hz, 2H). ¹⁹F NMR (400 MHz, Dimethylsulfoxide-d₆) δ −113.566.

### Example 444: 4-((7-(4-(2-(2-Aminopyridin-3-yl)-5-(4-fluorophenyl)-3H-imidazo[4,5-b]pyridin-3-yl)benzyl)-7-azaspiro[3.5]nonan-2-yl)amino)pyrimidine-2-carbonitrile

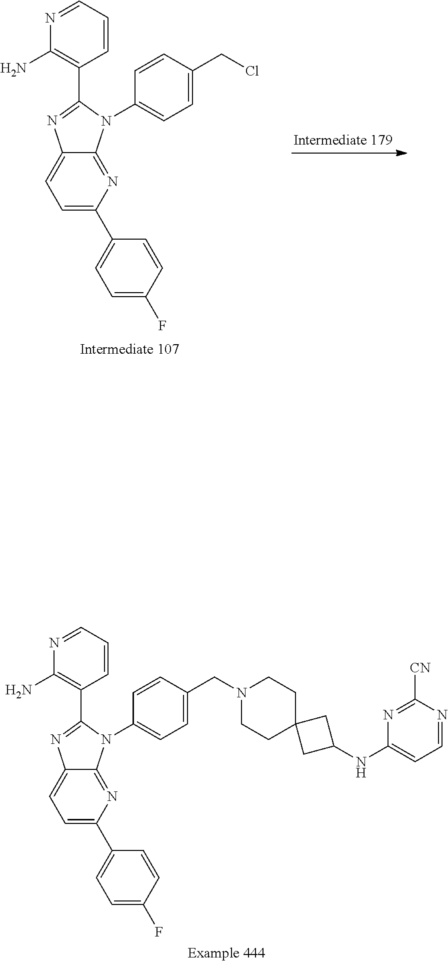 [SMILES: Nc1ncccc1-c1nc2ccc(-c3ccc(F)cc3)nc2n1-c1ccc(CCl)cc1 ; N#Cc1nccc(NC2CC3(CCN(Cc4ccc(-n5c(-c6cccnc6N)nc6ccc(-c7ccc(F)cc7)nc65)cc4)CC3)C2)n1]

To a solution of Intermediate 107 (200 mg, 465 μmol), Intermediate 179 (183 mg, 512 μmol, TFA salt) in DMF (2 mL) were added NaI (21 mg, 140 μmol) and K₂CO₃ (322 mg, 2.33 mmol). The mixture was stirred at 50° C. for 1 hr. The reaction mixture was poured into H₂O (10 mL) and extracted with EtOAc (15 mL×3). The organic phase was dried over anhydrous Na₂SO₄, filtered, and concentrated under reduced pressure. After purified by prep-HPLC (column: Daisogel SP ODS RPS 150×25 mm×5 μm; mobile phase: [water (NH₄HCO₃)-ACN]; gradient: 50%-80% B over 10 min), 4-((7-(4-(2-(2-aminopyridin-3-yl)-5-(4-fluorophenyl)-3H-imidazo[4,5-b]pyridin-3-yl)benzyl)-7-azaspiro[3.5]nonan-2-yl)amino)pyrimidine-2-carbonitrile (Example 444, 54 mg, yield: 18%) was obtained as an off white lyophilized powder. MS: m/z=637.4 [M+H]⁺. ¹H NMR (400 MHz, Dimethylsulfoxide-d₆) δ 8.44-8.31 (m, 1H), 8.27 (d, J=8.4 Hz, 1H), 8.14-8.02 (m, 3H), 8.01-7.93 (m, 2H), 7.50-7.39 (m, 4H), 7.29 (t, J=8.8 Hz, 2H), 7.19-7.11 (m, 1H), 7.04 (br s, 2H), 6.64 (d, J=6.0 Hz, 1H), 6.36 (dd, J=7.6, 4.8 Hz, 1H), 4.47-4.19 (m, 1H), 3.53 (s, 2H), 2.41-2.33 (m, 2H), 2.32-2.19 (m, 4H), 1.75-1.61 (m, 4H), 1.60-1.51 (m, 2H). ¹⁹F NMR (400 MHz, Dimethylsulfoxide-d₆) δ −113.573.

### Example 445: 4-((2-(4-(2-(2-Aminopyridin-3-yl)-5-(4-fluorophenyl)-3H-imidazo[4,5-b]pyridin-3-yl)benzyl)-2-azaspiro[3.3]heptan-6-yl)amino)pyrimidine-2-carbonitrile

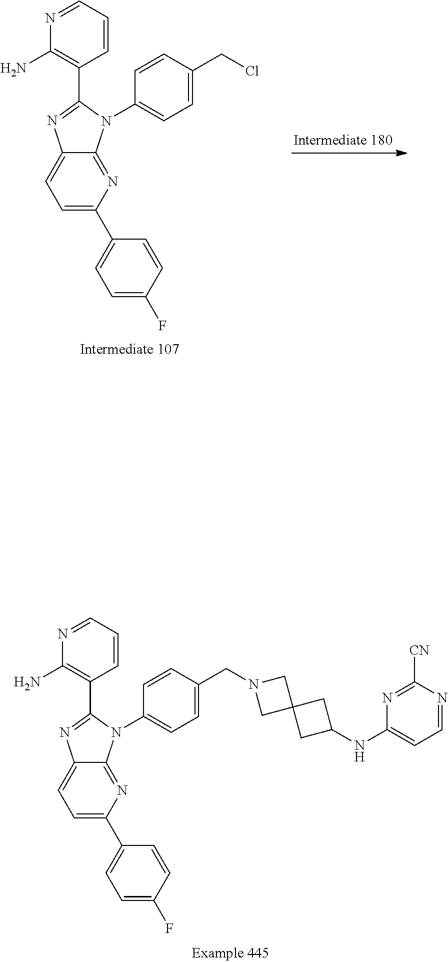 [SMILES: Nc1ncccc1-c1nc2ccc(-c3ccc(F)cc3)nc2n1-c1ccc(CCl)cc1 ; N#Cc1nccc(NC2CC3(C2)CN(Cc2ccc(-n4c(-c5cccnc5N)nc5ccc(-c6ccc(F)cc6)nc54)cc2)C3)n1]

To a solution of Intermediate 107 (200 mg, 465 μmol), Intermediate 180 (110 mg, 512 μmol, TFA salt) in DMF (2 mL) were added NaI (21 mg, 140 μmol) and K₂CO₃ (322 mg, 2.33 mmol). The mixture was stirred at 50° C. for 1 hr. The reaction mixture was poured into H₂O (10 mL), extracted with EtOAc (15 mL×3). The organic phase was dried over anhydrous Na₂SO₄, filtered, and concentrated under reduced pressure. After purified by prep-HPLC (column: Waters xbridge 150×25 mm 10 μm; mobile phase: [water (NH₄HCO₃)-ACN]; gradient: 42%-72% B over 10 min), 4-((2-(4-(2-(2-aminopyridin-3-yl)-5-(4-fluorophenyl)-3H-imidazo[4,5-b]pyridin-3-yl)benzyl)-2-azaspiro[3.3]heptan-6-yl)amino)pyrimidine-2-carbonitrile (Example 445, 45.1 mg, yield: 16%) was obtained as an off white lyophilized powder. MS: m/z=609.3 [M+H]⁺. ¹H NMR (400 MHz, Dimethylsulfoxide-d₆) δ 8.43-8.29 (m, 1H), 8.26 (d, J=8.4 Hz, 1H), 8.08 (dd, J=8.8, 5.6 Hz, 3H), 8.00 (dd, J=4.8, 1.6 Hz, 1H), 7.97 (d, J=8.4 Hz, 1H), 7.51-7.36 (m, 4H), 7.30 (t, J=8.4 Hz, 2H), 7.15 (dd, J=7.6, 1.6 Hz, 1H), 7.00 (br s, 2H), 6.63 (d, J=6.0 Hz, 1H), 6.40 (dd, J=7.6, 4.4 Hz, 1H), 4.35-4.14 (m, 1H), 3.60 (s, 2H), 3.30-3.25 (m, 2H), 3.20-3.05 (m, 2H), 2.58-2.52 (m, 2H), 2.20-1.94 (m, 2H). ¹⁹F NMR (400 MHz, Dimethylsulfoxide-d₆) δ −113.587.

### Example 446: 4-((2-(4-(2-(2-Aminopyridin-3-yl)-5-(4-fluorophenyl)-3H-imidazo[4,5-b]pyridin-3-yl)benzyl)-2-azaspiro[3.5]nonan-7-yl)amino)pyrimidine-2-carbonitrile

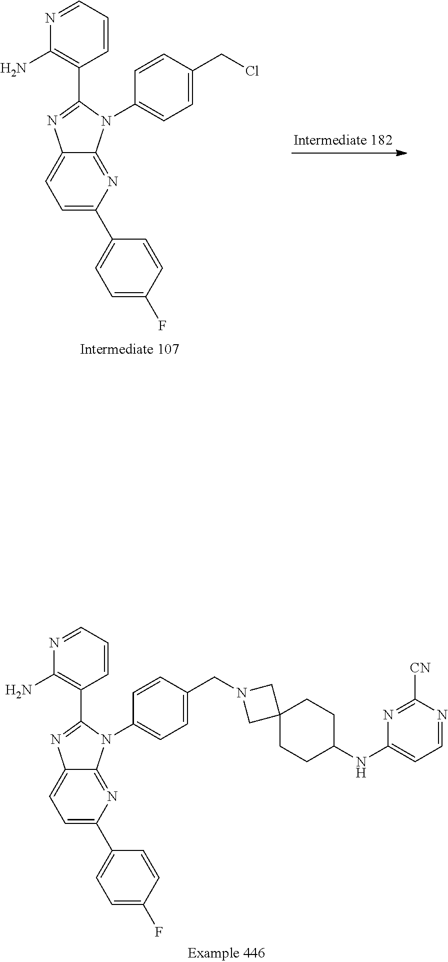 [SMILES: Nc1ncccc1-c1nc2ccc(-c3ccc(F)cc3)nc2n1-c1ccc(CCl)cc1 ; N#Cc1nccc(NC2CCC3(CC2)CN(Cc2ccc(-n4c(-c5cccnc5N)nc5ccc(-c6ccc(F)cc6)nc54)cc2)C3)n1]

To a solution of Intermediate 107 (200 mg, 465 μmol), Intermediate 182 (125 mg, 348 μmol, TFA salt) in DMF (2 mL) were added NaI (21 mg, 140 μmol) and K₂CO₃ (322 mg, 2.33 mmol). The mixture was stirred at 50° C. for 1 hr. The reaction mixture was poured into H₂O (15 mL), extracted with EtOAc (15 mL×3). The organic phase was dried over anhydrous Na₂SO₄, filtered, and concentrated under reduced pressure. After purified by prep-HPLC (column: Waters xbridge 150×25 mm×10 μm; mobile phase: [water (NH₄HCO₃)-ACN]; gradient: 40%-70% B over 11 min), 4-((2-(4-(2-(2-aminopyridin-3-yl)-5-(4-fluorophenyl)-3H-imidazo[4,5-b]pyridin-3-yl)benzyl)-2-azaspiro[3.5]nonan-7-yl)amino)pyrimidine-2-carbonitrile (Example 446, 37.2 mg, yield: 13%) was obtained as a yellow lyophilized powder. MS: m/z=637.4 [M+H]⁺. ¹H NMR (400 MHz, Dimethylsulfoxide-d₆) δ 8.27 (d, J=8.4 Hz, 1H), 8.12-8.03 (m, 3H), 8.02-7.92 (m, 3H), 7.51-7.38 (m, 4H), 7.30 (t, J=8.8 Hz, 2H), 7.19-7.12 (m, 1H), 6.99 (br s, 2H), 6.65 (d, J=6.0 Hz 1H), 6.39 (dd, J=7.6, 4.8 Hz, 1H), 3.85-3.74 (m, 1H), 3.67 (s, 2H), 3.06-2.89 (m, 4H), 1.95-1.87 (m, 2H), 1.84-1.74 (m, 2H), 1.60-1.50 (m, 2H), 1.27-1.22 (m, 2H). ¹⁹F NMR (400 MHz, Dimethylsulfoxide-d₆) δ −113.580.

### Example 447: 4-(6-(4-(2-(2-Aminopyridin-3-yl)-5-(4-fluorophenyl)-3H-imidazo[4,5-b]pyridin-3-yl)benzyl)-2,6-diazaspiro[3.3]heptan-2-yl)pyrimidine-2-carbonitrile

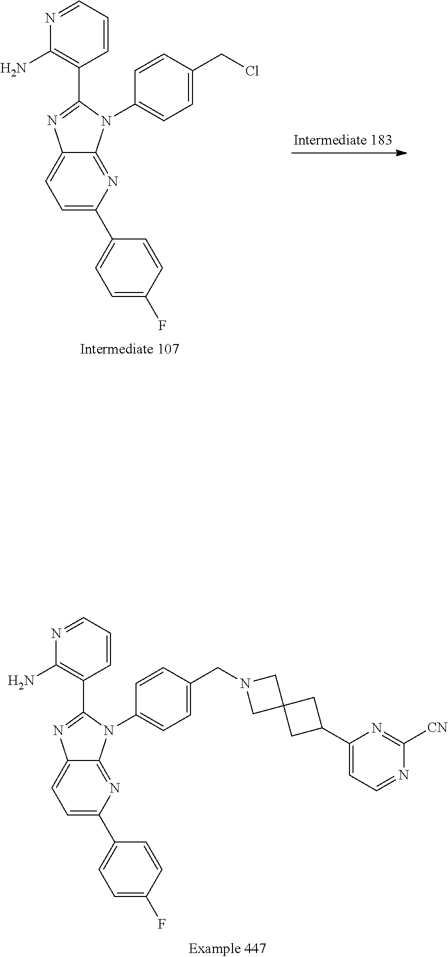 [SMILES: Nc1ncccc1-c1nc2ccc(-c3ccc(F)cc3)nc2n1-c1ccc(CCl)cc1 ; N#Cc1nccc(C2CC3(C2)CN(Cc2ccc(-n4c(-c5cccnc5N)nc5ccc(-c6ccc(F)cc6)nc54)cc2)C3)n1]

To a solution of Intermediate 107 (200 mg, 465 μmol), Intermediate 183 (161 mg, 512 μmol, TFA salt) in DMF (2 mL) were added NaI (70 mg, 465 μmol) and K₂CO₃ (322 mg, 2.33 mmol). The mixture was stirred at 50° C. for 1 hr. The reaction mixture was poured into H₂O (15 mL), extracted with EtOAc (15 mL×3). The organic phase was dried over anhydrous Na₂SO₄, filtered, and concentrated under reduced pressure. After purified by prep-HPLC (column: Waters xbridge 150×25 mm 10 μm; mobile phase: [water (NH₄HCO₃)-ACN]; gradient: 45%-75% B over 10 min), 4-(6-(4-(2-(2-aminopyridin-3-yl)-5-(4-fluorophenyl)-3H-imidazo[4,5-b]pyridin-3-yl)benzyl)-2,6-diazaspiro[3.3]heptan-2-yl)pyrimidine-2-carbonitrile (Example 447, 52.5 mg, yield: 19%) was obtained as a yellow lyophilized powder. MS: m/z=595.3 [M+H]⁺. ¹H NMR (400 MHz, Dimethylsulfoxide-d₆) δ 8.26 (d, J=8.4 Hz, 1H), 8.22 (d, J=6.0 Hz, 1H), 8.07 (dd, J=8.4, 5.6 Hz, 2H), 8.01-7.94 (m, 2H), 7.43 (s, 4H), 7.29 (t, J=8.8 Hz, 2H), 7.17-7.10 (m, 1H), 7.05-6.92 (m, 2H), 6.70-6.52 (m, 1H), 6.39 (dd, J=7.6, 4.8 Hz, 1H), 4.26-4.15 (m, 4H), 3.63 (s, 2H), 3.42-3.35 (s, 4H). ¹⁹F NMR (400 MHz, Dimethylsulfoxide-d₆) δ −113.581.

### Example 448: 4-(8-(4-(2-(2-Aminopyridin-3-yl)-5-(4-fluorophenyl)-3H-imidazo[4,5-b]pyridin-3-yl)benzyl)-3,8-diazabicyclo[3.2.1]octan-3-yl)pyri midine-2-carbonitrile

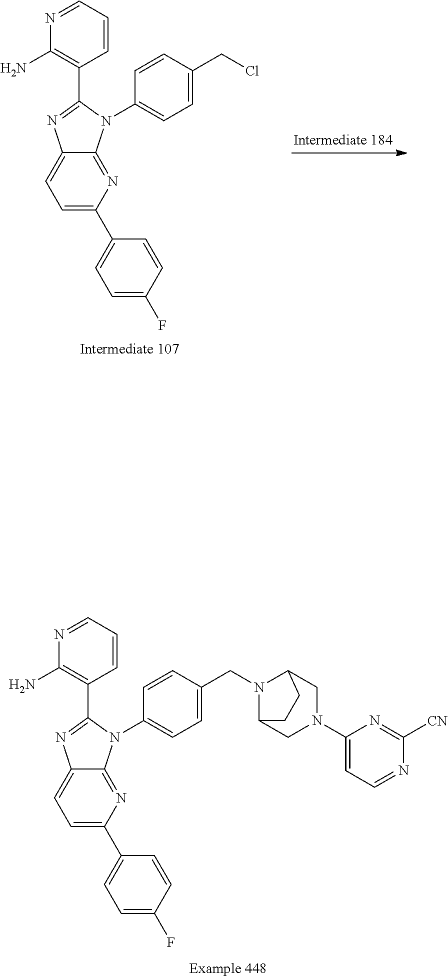 [SMILES: Nc1ncccc1-c1nc2ccc(-c3ccc(F)cc3)nc2n1-c1ccc(CCl)cc1 ; N#Cc1nccc(N2CC3CCC(C2)N3Cc2ccc(-n3c(-c4cccnc4N)nc4ccc(-c5ccc(F)cc5)nc43)cc2)n1]

To a solution of Intermediate 107 (200 mg, 465 μmol), Intermediate 184 (169 mg, 512 μmol, TFA salt) in DMF (2 mL) were added NaI (21 mg, 140 μmol) and K₂CO₃ (322 mg, 2.33 mmol). The mixture was stirred at 50° C. for 1 hr. The reaction mixture was poured into H₂O (10 mL), extracted with EtOAc (15 mL×3). The organic phase was dried over anhydrous Na₂SO₄, filtered, and concentrated under reduced pressure. After purified by prep-HPLC (column: Daisogel SP ODS RPS 150×25 mm×5 μm; mobile phase: [water (NH₄HCO₃)-ACN]; gradient: 54%-84% B over 10 min), 4-(8-(4-(2-(2-aminopyridin-3-yl)-5-(4-fluorophenyl)-3H-imidazo[4,5-b]pyridin-3-yl)benzyl)-3,8-diazabicyclo[3.2.1]octan-3-yl)pyrimidine-2-carbonitrile (Example 448, 45.7 mg, yield: 16%) was obtained as an off white lyophilized powder. MS: m/z=609.2 [M+H]⁺. ¹H NMR (400 MHz, Dimethylsulfoxide-d₆) δ 8.33-8.20 (m, 2H), 8.08 (dd, J=8.8, 5.6 Hz, 2H), 8.00 (dd, J=4.8, 1.6 Hz, 1H), 7.97 (d, J=8.4 Hz, 1H), 7.58 (d, J=8.4 Hz, 2H), 7.46 (d, J=8.0 Hz, 2H), 7.30 (t, J=8.8 Hz, 2H), 7.17 (dd, J=7.6, 1.6 Hz, 1H), 7.08-6.95 (m, 3H), 6.41 (dd, J=7.6, 4.8 Hz, 1H), 4.44-4.11 (m, 1H), 3.84-3.57 (m, 3H), 3.33 (s, 2H), 3.26-3.03 (m, 2H), 2.06-1.97 (m, 2H), 1.65-1.42 (m, 2H). ¹⁹F NMR (400 MHz, Dimethylsulfoxide-d₆) δ −113.573.

### Example 449: 4-((3aR,6aS)-5-(4-(2-(2-Aminopyridin-3-yl)-5-(4-fluorophenyl)-3H-imidazo[4,5-b]pyridin-3-yl)benzyl)hexahydropyrrolo[3,4-c]pyrrol-2(1H)-yl)pyrimidine-2-carbonitrile

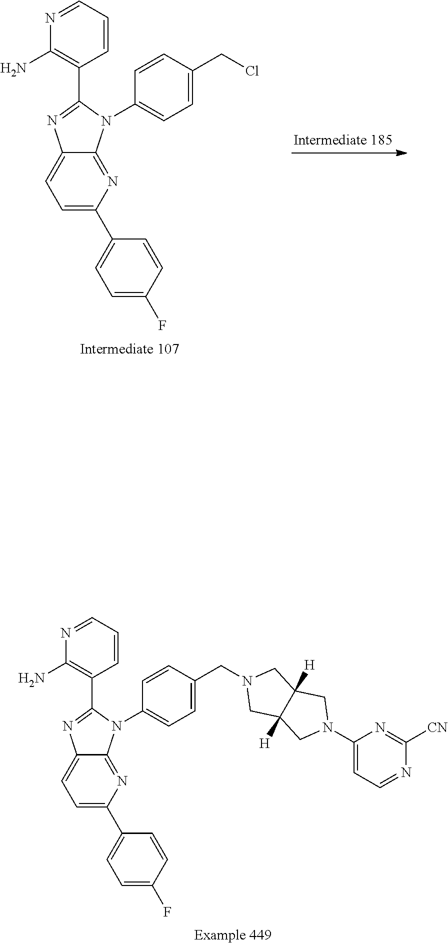 [SMILES: Nc1ncccc1-c1nc2ccc(-c3ccc(F)cc3)nc2n1-c1ccc(CCl)cc1 ; N#Cc1nccc(N2C[C@H]3CN(Cc4ccc(-n5c(-c6cccnc6N)nc6ccc(-c7ccc(F)cc7)nc65)cc4)C[C@H]3C2)n1]

To a solution of Intermediate 107 (240 mg, 558 μmol), Intermediate 185 (202 mg, 614 μmol, TFA salt) in DMF (2 mL) were added NaI (25 mg, 167 μmol) and K₂CO₃ (386 mg, 2.79 mmol). The mixture was stirred at 50° C. for 1 hr. The reaction mixture was poured into H₂O (10 mL), extracted with EtOAc (15 mL×3). The organic phase was dried over anhydrous Na₂SO₄, filtered and concentrated under reduced pressure. After purified by prep-HPLC (column: Waters Xbridge BEH C18 150×25 mm×5 μm; mobile phase: [water (NH₄HCO₃)-ACN]; gradient: 50%-80% B over 10 min), 4-((3aR,6aS)-5-(4-(2-(2-aminopyridin-3-yl)-5-(4-fluorophenyl)-3H-imidazo[4,5-b]pyridin-3-yl)benzyl)hexahydropyrrolo[3,4-c]pyrrol-2(1H)-yl)pyrimidine-2-carbonitrile (Example 449, 57.2 mg, yield: 17%) was obtained as an off white lyophilized powder. MS: m/z=609.3 [M+H]⁺. ¹H NMR (400 MHz, Dimethylsulfoxide-d₆) δ 8.34-8.16 (m, 2H), 8.07 (dd, J=8.8, 5.6 Hz, 2H), 8.02-7.94 (m, 2H), 7.54-7.35 (m, 4H), 7.29 (t, J=8.8 Hz, 2H), 7.14 (dd, J=7.6, 1.6 Hz, 1H), 7.01 (s, 2H), 6.79 (d, J=6.0 Hz, 1H), 6.37 (dd, J=7.6, 4.8 Hz, 1H), 3.87-3.73 (m, 1H), 3.68 (s, 2H), 3.53-3.42 (m, 1H), 3.04-2.90 (m, 2H), 2.71-2.52 (m, 6H). ¹⁹F NMR (400 MHz, Dimethylsulfoxide-d₆) δ −113.558.

### Example 450: 4-((3aS,6aS)-5-(4-(2-(2-Aminopyridin-3-yl)-5-(4-fluorophenyl)-3H-imidazo[4,5-b]pyridin-3-yl)benzyl)hexahydropyrrolo[3,4-c]pyrrol-2(1H)-yl)pyrimidine-2-carbonitrile

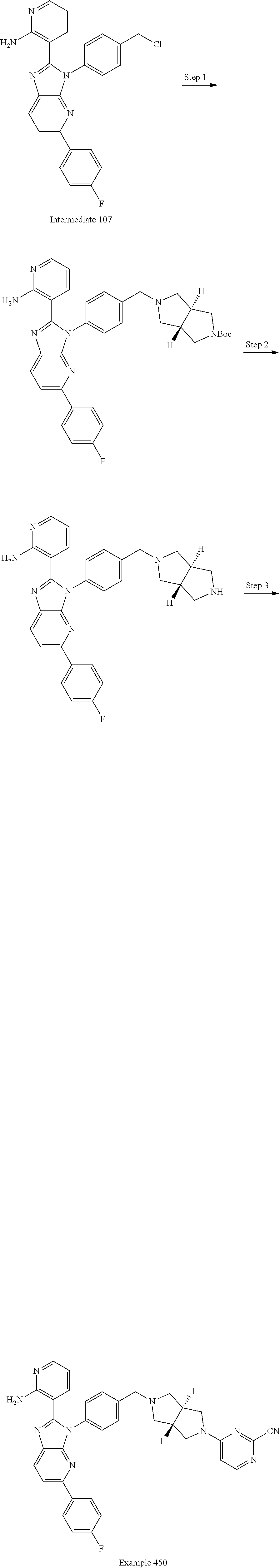 [SMILES: Nc1ncccc1-c1nc2ccc(-c3ccc(F)cc3)nc2n1-c1ccc(CCl)cc1 ; CC(C)(C)OC(=O)N1C[C@@H]2CN(Cc3ccc(-n4c(-c5cccnc5N)nc5ccc(-c6ccc(F)cc6)nc54)cc3)C[C@H]2C1 ; Nc1ncccc1-c1nc2ccc(-c3ccc(F)cc3)nc2n1-c1ccc(CN2C[C@H]3CNC[C@@H]3C2)cc1 ; N#Cc1nccc(N2C[C@@H]3CN(Cc4ccc(-n5c(-c6cccnc6N)nc6ccc(-c7ccc(F)cc7)nc65)cc4)C[C@H]3C2)n1]

### Step 1 Tert-butyl (3aS,6aS)-5-(4-(2-(2-aminopyridin-3-yl)-5-(4-fluorophenyl)-3H-imidazo[4,5-b]pyridin-3-yl)benzyl)hexahydropyrrolo[3,4-c]pyrrole-2(1H)-carboxylate

To a solution of Intermediate 107 (200 mg, 465 μmol), tert-butyl (3aS,6aS)-hexahydropyrrolo[3,4-c]pyrrole-2(1H)-carboxylate (109 mg, 512 μmol) in DMF (2 mL) were added K₂CO₃ (129 mg, 931 μmol) and NaI (6.97 mg, 46.5 μmol). The mixture was stirred at 80° C. for 2 hr. The reaction mixture was poured into H₂O (15 mL), extracted with EtOAc (15 mL×3). The organic phase was dried over anhydrous Na₂SO₄, filtered, and concentrated under reduced pressure. After purified by silica gel flash chromatography (Eluent of 0%˜52% EtOAc in petroleum ether), tert-butyl (3aS,6aS)-5-(4-(2-(2-aminopyridin-3-yl)-5-(4-fluorophenyl)-3H-imidazo[4,5-b]pyridin-3-yl)benzyl)hexahydropyrrolo[3,4-c]pyrrole-2(1H)-carboxylate (190 mg, yield: 67%) was obtained as a yellow oil. MS: m/z=606.1 [M+H]⁺.

### Step 2: 3-(5-(4-Fluorophenyl)-3-(4-(((3aR,6aR)-hexahydropyrrolo[3,4-c]pyrrol-2(1H)-yl)methyl)phenyl)-3H-imidazo[4,5-b]pyridin-2-yl)pyridin-2-amine

To a solution of (3aS,6aS)-5-(4-(2-(2-aminopyridin-3-yl)-5-(4-fluorophenyl)-3H-imidazo[4,5-b]pyridin-3-yl)benzyl)hexahydropyrrolo[3,4-c]pyrrole-2(1H)-carboxylate (190 mg, 314 μmol) in 1,4-dioxane (2 mL) was added HCl in 1,4-dioxane (4 M, 2 mL). The mixture was stirred at 25° C. for 2 hr. The reaction mixture was concentrated directly. 3-(5-(4-fluorophenyl)-3-(4-(((3aR,6aR)-hexahydropyrrolo[3,4-c]pyrrol-2(1H)-yl)methyl)phenyl)-3H-imidazo[4,5-b]pyridin-2-yl)pyridin-2-amine (160 mg, HCl salt, yield: 94%) was obtained as a yellow solid. ¹H NMR (400 MHz, Dimethylsulfoxide-d₆) δ 12.29 (s, 1H), 10.05 (s, 1H), 9.78 (s, 1H), 8.37 (d, J=8.4 Hz, 2H), 8.17-8.02 (m, 4H), 7.93-7.79 (m, 3H), 7.66 (d, J=8.4 Hz, 2H), 7.32 (t, J=8.8 Hz, 2H), 6.91 (dd, J=7.2, 6.4 Hz, 1H), 4.70-4.49 (m, 2H), 3.50-3.41 (m, 2H), 3.40-3.28 (m, 4H), 3.18-3.08 (m, 1H), 3.05-2.91 (m, 2H), 2.90-2.75 (m, 1H), 2.45-2.32 (m, 2H).

### Step 3: 4-((3aS,6aS)-5-(4-(2-(2-Aminopyridin-3-yl)-5-(4-fluorophenyl)-3H-imidazo[4,5-b]pyridin-3-yl)benzyl)hexahydropyrrolo[3,4-c]pyrrol-2(1H)-yl)pyrimidine-2-carbonitrile

To a solution of 3-(5-(4-fluorophenyl)-3-(4-(((3aR,6aR)-hexahydropyrrolo[3,4-c]pyrrol-2(1H)-yl)methyl)phenyl)-3H-imidazo[4,5-b]pyridin-2-yl)pyridin-2-amine (113 mg, 223.50 μmol, HCl salt), 4-chloropyrimidine-2-carbonitrile (37.4 mg, 268 μmol) in DMF (2 mL) were added K₂CO₃ (92.7 mg, 671 μmol) and NaI (3.35 mg, 22.4 μmol). The mixture was stirred at 25° C. for 1 hr. The reaction mixture was poured into H₂O (15 mL), extracted with EtOAc (15 mL×3). The organic phase was dried over Na₂SO₄, filtered and concentrated under reduced pressure. After purified by prep-HPLC (column: Phenomenex C18 150×25 mm×10 μm; mobile phase: [water (NH₄HCO₃)-ACN]; gradient: 50%-80% B over 11 min), 4-((3aS,6aS)-5-(4-(2-(2-aminopyridin-3-yl)-5-(4-fluorophenyl)-3H-imidazo[4,5-b]pyridin-3-yl)benzyl)hexahydropyrrolo[3,4-c]pyrrol-2(1H)-yl)pyrimidine-2-carbonitrile (Example 450, 10 mg, yield: 7%) was obtained as a light yellow lyophilized powder. MS: m4=609.4 [M+H]⁺. ¹H NMR (400 MHz, Dimethylsulfoxide-d₆) δ 8.31-8.20 (m, 2H), 6.91 (dd, J=8.8, 5.6 Hz, 2H), 8.04-7.92 (m, 2H), 7.51 (d, J=8.4 Hz, 2H), 7.44 (d, J=8.0 Hz, 2H), 7.30 (t, J=8.8 Hz, 2H), 7.16 (dd, J=8.0, 1.6 Hz, 1H), 7.01 (br s, 2H), 6.75 (d, J=6.4 Hz, 1H), 7.16 (dd, J=8.0, 5.2 Hz, 1H), 4.04-3.90 (m, 2H), 3.88-3.78 (m, 1H), 3.67-3.56 (m, 1H), 3.15-3.07 (m, 2H), 2.97-2.87 (m, 2H), 2.71-2.64 (m, 2H), 2.41-2.31 (m, 2H). ¹⁹F NMR (400 MHz, Dimethylsulfoxide-d₆) δ −113.580.

### Example 451: 4-((1-(4-(2-(2-Aminopyridin-3-yl)-5-(4-fluorophenyl)-3H-imidazo[4,5-b]pyridin-3-yl)benzyl)piperidin-4-yl-4-d)amino)pyrimidine-2-carbonitrile

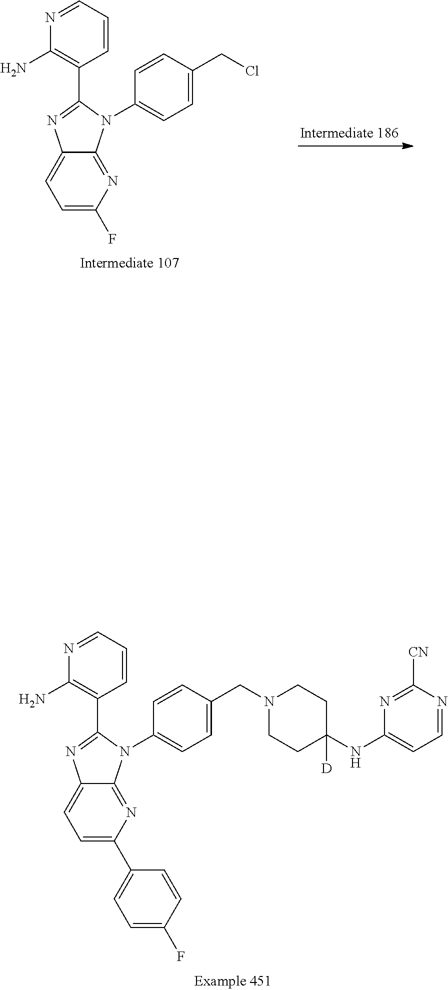 [SMILES: Nc1ncccc1-c1nc2ccc(F)nc2n1-c1ccc(CCl)cc1 ; [2H]C1(Nc2ccnc(C#N)n2)CCN(Cc2ccc(-n3c(-c4cccnc4N)nc4ccc(-c5ccc(F)cc5)nc43)cc2)CC1]

To a solution of Intermediate 107 (80 mg, 186 μmol), Intermediate 186 (38 mg, 186 μmol) in DMF (2 mL) were added NaI (5.58 mg, 37.2 μmol) and K₂CO₃ (77.2 mg, 558 μmol). The mixture was stirred at 50° C. for 1 hr. The mixture was diluted with H₂O (5 mL) and extracted with CH₂Cl₂ (5 mL×3). The combined organic layers were washed with brine (5 mL×2), dried over Na₂SO₄, filtered, and concentrated under reduced pressure. After purified by silica gel flash chromatography (Eluent of 5˜6% MeOH in CH₂Cl₂), 4-((1-(4-(2-(2-Aminopyridin-3-yl)-5-(4-fluorophenyl)-3H-imidazo[4,5-b]pyridin-3-yl)benzyl)piperidin-4-yl-4-d)amino)pyrimidine-2-carbonitrile (Example 451, 49.5 mg, yield: 42%) was obtained as a yellow solid. MS: m/z=598.7 [M+H]⁺. ¹H NMR (400 MHz, Dimethylsulfoxide-d₆) δ 8.27 (d, J=8.4 Hz, 1H), 8.11-8.03 (m, 4H), 8.01-7.96 (m, 2H), 7.47 (q, J=8.4 Hz, 4H), 7.29 (t, J=8.8 Hz, 2H), 7.15 (dd, J=7.6, 2.0 Hz, 1H), 7.03 (s, 2H), 6.67 (br d, J=6.0 Hz, 1H), 6.37 (dd, J=7.6, 4.8 Hz, 1H), 3.59 (s, 2H), 2.85-2.80 (m, 2H), 2.16 (t, J=10.4 Hz, 2H), 1.90-1.86 (m, 2H), 1.52-1.46 (m, 2H). ¹⁹F NMR (400 MHz, Dimethylsulfoxide-d₆) δ −113.6.

### Example 452: 4-((1-(4-(2-(2-Aminopyridin-3-yl)-5,6-dimethyl-3H-imidazo[4,5-b]pyridin-3-yl)benzyl)piperidin-4-yl-4-d)amino)pyrimidine-2-carbonitrile

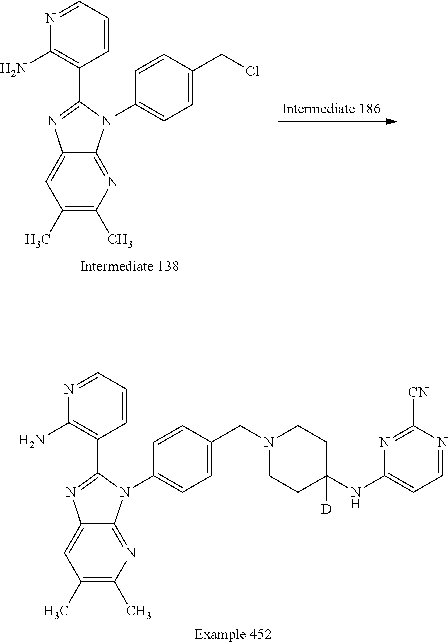 [SMILES: Cc1cc2nc(-c3cccnc3N)n(-c3ccc(CCl)cc3)c2nc1C ; [2H]C1(Nc2ccnc(C#N)n2)CCN(Cc2ccc(-n3c(-c4cccnc4N)nc4cc(C)c(C)nc43)cc2)CC1]

To a solution of Intermediate 138 (100 mg, 275 μmol) and Intermediate 186 (56 mg, 275 μmol) in DMF (3 mL) were added NaI (8.24 mg, 55 μmol) and K₂CO₃ (114 mg, 825 μmol). The mixture was stirred at 50° C. for 1 hr. The mixture was diluted with H₂O (5 mL) and extracted with CH₂Cl₂ (5 mL×3). The combined organic layers were washed with brine (5 mL×2), dried over Na₂SO₄, filtered, and concentrated under reduced pressure. After purified by silica gel flash chromatography (Eluent of 5˜6% MeOH in CH₂Cl₂) and then purified by prep-TLC (MeOH in CH₂Cl₂=1:10), 4-((1-(4-(2-(2-Aminopyridin-3-yl)-5,6-dimethyl-3H-imidazo[4,5-b]pyridin-3-yl)benzyl)piperidin-4-yl-4-d)amino)pyrimidine-2-carbonitrile (Example 452, 12.3 mg, yield: 17%) was obtained as a yellow solid. MS: m/z=532.7 [M+H]⁺. ¹H NMR (400 MHz, Methanol-d₄) δ 8.00 (d, J=5.6 Hz, 1H), 7.94 (dd, J=5.2, 1.6 Hz, 1H), 7.89 (s, 1H), 7.51 (d, J=8.4 Hz, 2H), 7.35 (d, J=8.4 Hz, 2H), 7.23 (dd, J=7.6, 1.6 Hz, 1H), 6.59 (d, J=5.6 Hz, 1H), 6.43 (dd, J=7.6, 5.2 Hz, 1H), 3.65 (s, 2H), 2.95 (d, J=11.6 Hz, 2H), 2.53 (s, 3H), 2.44 (s, 3H), 2.27 (t, J=11.2 Hz, 2H), 1.99 (d, J=12.8 Hz, 2H), 1.64-1.56 (m, 2H).

### Example 453: 4-((1-(4-(2-(2-Aminopyridin-3-yl)-5-(cyclopent-1-en-1-yl)-3H-imidazo[4,5-b]pyridin-3-yl)benzyl)piperidin-4-yl)amino)pyrimidine-2-carbonitrile

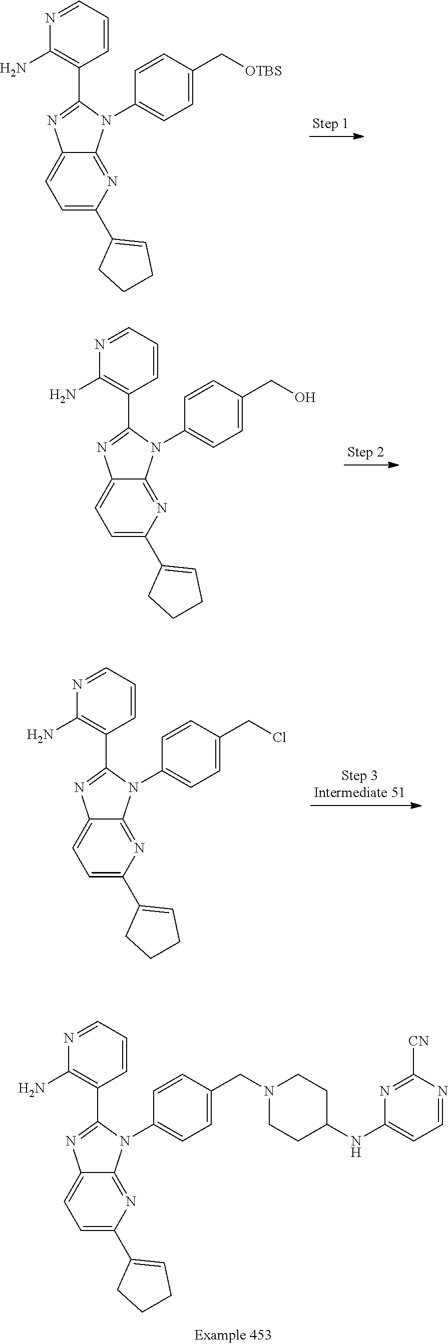

### Step 1 (4-(2-(2-Aminopyridin-3-yl)-5-(cyclopent-1-en-1-yl)-3H-imidazo[4,5-b]pyridin-3-yl)phenyl)methanol

A mixture of 3-(3-(4-(((tert-butyldimethylsilyl)oxy)methyl)phenyl)-5-(cyclopent-1-en-1-yl)-3H-imidazo[4,5-b]pyridin-2-yl)pyridin-2-amine (refer to Intermediate 118 for detail procedures, 275 mg, 553 μmol) in TBAF (2.5 M in THF, 4 mL) was degassed and purged with N₂ three times. The mixture was stirred at 25° C. for 0.5 hr under N₂ atmosphere. The reaction mixture was poured into H₂O (15 mL), extracted with EtOAc (15 mL×3). The organic phase was dried over anhydrous Na₂SO₄, filtered and concentrated under reduced pressure, (4-(2-(2-aminopyridin-3-yl)-5-(cyclopent-1-en-1-yl)-3H-imidazo[4,5-b]pyridin-3-yl)phenyl)methanol (220 mg, crude) was obtained as a gray solid. MS: m/z=384.1 [M+H]⁺.

### Step 2: 3-(3-(4-(Chloromethyl)phenyl)-5-(cyclopent-1-en-1-yl)-3H-imidazo[4,5-b]pyridin-2-yl)pyridin-2-amine

To a solution of (4-(2-(2-aminopyridin-3-yl)-5-(cyclopent-1-en-1-yl)-3H-imidazo[4,5-b]pyridin-3-yl)phenyl)methanol (220 mg, 574 μmol) in CH₂Cl₂ (4 mL) was added SOCl₂ (68 mg, 574 μmol). The mixture was stirred at 45° C. for 0.3 hr. The mixture was concentrated under reduced pressure, 3-(3-(4-(chloromethyl)phenyl)-5-(cyclopent-1-en-1-yl)-3H-imidazo[4,5-b]pyridin-2-yl)pyridin-2-amine (251 mg, crude) was obtained as a yellow solid. MS: m/z=402.0 [M+H]⁺.

### Step 3: 4-((1-(4-(2-(2-Aminopyridin-3-yl)-5-(cyclopent-1-en-1-yl)-3H-imidazo[4,5-b]pyridin-3-yl)benzyl)piperidin-4-yl)amino)pyrimidine-2-carbonitrile

To a solution of 3-(3-(4-(chloromethyl)phenyl)-5-(cyclopent-1-en-1-yl)-3H-imidazo[4,5-b]pyridin-2-yl)pyridin-2-amine (251 mg, 625 μmol) and Intermediate 51 (218 mg, 687 μmol) in DMF (2 mL) were added K₂CO₃ (432 mg, 3.1 mmol) and NaI (28.1 mg, 187 μmol). The mixture was stirred at 50° C. for 1 hr. The reaction mixture was poured into H₂O (15 mL), extracted with EtOAc (15 mL×3). The organic phase was dried over Na₂SO₄, filtered, and concentrated under reduced pressure. After purified by silica gel flash chromatography (Eluent of 0˜15% MeOH in CH₂Cl₂), 4-((1-(4-(2-(2-aminopyridin-3-yl)-5-(cyclopent-1-en-1-yl)-3H-imidazo[4,5-b]pyridin-3-yl)benzyl)piperidin-4-yl)amino)pyrimidine-2-carbonitrile (Example 453, 107.8 mg, yield: 30%) was obtained as an off white solid. MS: m/z=569.3 [M+H]^{+ 1}H NMR (400 MHz, Dimethylsulfoxide-d₆) δ 8.26-8.03 (m, 3H), 7.98 (dd, J=4.8, 2.0 Hz, 1H), 7.57 (d, J=8.4 Hz, 1H), 7.48-7.42 (m, 2H), 7.40-7.33 (m, 2H), 7.11 (dd, J=8.0, 2.0 Hz, 1H), 7.03 (br s, 2H), 6.67 (d, J=6.0 Hz, JH), 6.53 (t, J=1.6 Hz, 1H), 6.36 (dd, J=7.6, 4.8 Hz, 1H), 3.89-3.71 (m, 1H), 3.57 (s, 2H), 3.35-3.34 (m, 2H), 2.86-2.76 (m, 2H), 2.71-2.65 (m, 2H), 2.20-2.10 (m, 2H), 1.99-1.83 (m, 4H), 1.54-1.44 (m, 2H).

### Example 454: 4-((1-(4-(2-(2-Aminopyridin-3-yl)-5-vinyl-3H-imidazo[4,5-b]pyridin-3-yl)benzyl)piperidin-4-yl)amino)pyrimidine-2-carbonitrile

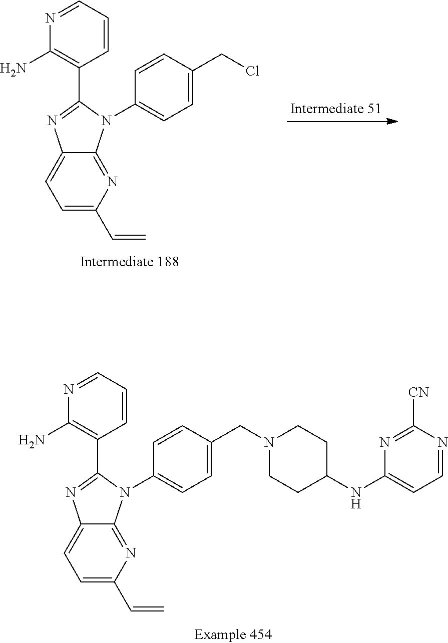 [SMILES: C=Cc1ccc2nc(-c3cccnc3N)n(-c3ccc(CCl)cc3)c2n1 ; C=Cc1ccc2nc(-c3cccnc3N)n(-c3ccc(CN4CCC(Nc5ccnc(C#N)n5)CC4)cc3)c2n1]

To a solution of Intermediate 188 (240 mg, 602 μmol, HCl) and Intermediate 51 (191 mg, 602 μmol, TFA) in DMF (2 mL) was added NaI (45.1 mg, 302 μmol) and K₂CO₃ (416 mg, 3.01 mmol). The mixture was stirred at 50° C. for 1 hr. The reaction mixture was diluted with water (10 mL) and extracted with EtOAc (2×20 mL). The combined organic layers were washed with brine (30 mL), dried over Na₂SO₄, filtered, and concentrated under reduced pressure. After purified by silica gel flash chromatography (Eluent of 0˜7% MeOH in CH₂Cl₂), 4-((1-(4-(2-(2-aminopyridin-3-yl)-5-vinyl-3H-imidazo[4,5-b]pyridin-3-yl)benzyl)piperidin-4-yl)amino)pyrimidine-2-carbonitrile (Example 454, 145.4 mg, yield: 44%) was obtained as an off white solid. MS: m/z=529.2 [M+H]⁺. ¹H NMR (400 MHz, Dimethylsulfoxide-d₆) δ 8.16 (d, J=8.4 Hz, 1H), 8.07 (dd, J=13.6, 6.0 Hz, 2H), 7.98 (dd, J=4.4, 1.2 Hz, 1H), 7.57 (d, J=8.0 Hz, 1H), 7.50-7.43 (m, 2H), 7.42-7.34 (m, 2H), 7.12 (dd, J=8.0, 1.6 Hz, 1H), 7.02 (br s, 2H), 6.85 (dd, J=17.6, 10.4 Hz, 1H), 6.67 (br d, J=5.6 Hz, 1H), 6.36 (dd, J=7.6, 4.8 Hz, 1H), 6.11 (d, J=16.8 Hz, 1H), 5.40 (d, J=12.0 Hz, 1H), 3.89-3.74 (m, 1H), 3.58 (s, 2H), 2.82 (d, J=10.4 Hz, 2H), 2.23-2.09 (m, 2H), 1.95-1.82 (m, 2H), 1.57-1.42 (m, 2H).

### Example 455: 4-((1-(4-(2-(2-Aminopyridin-3-yl)-5,6-dimethyl-3H-imidazo[4,5-b]pyridin-3-yl)benzyl)piperidin-4-yl)(methyl-d₃)amino)pyrimidine-2-carbonitrile

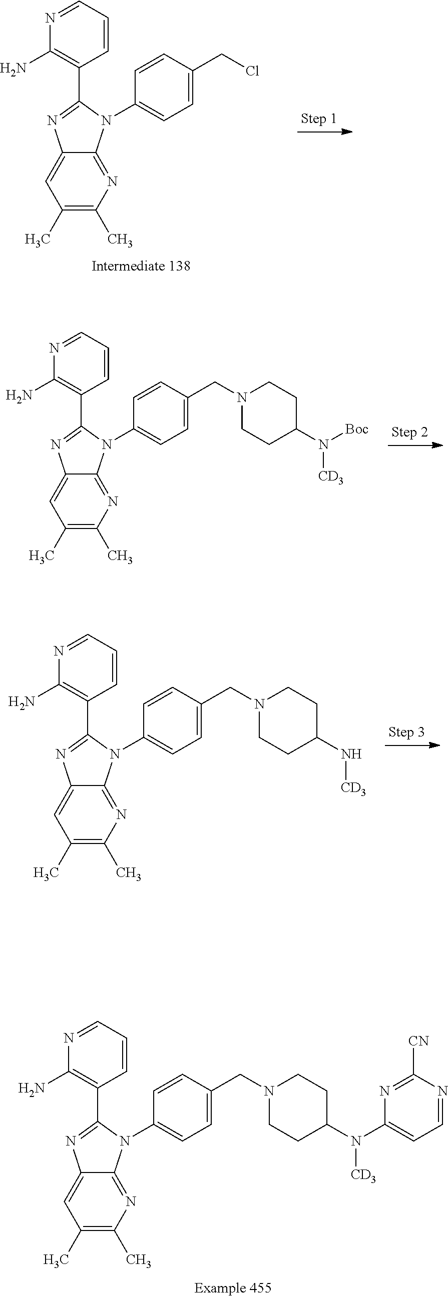 [SMILES: Cc1cc2nc(-c3cccnc3N)n(-c3ccc(CCl)cc3)c2nc1C ; [2H]C([2H])([2H])N(C(=O)OC(C)(C)C)C1CCN(Cc2ccc(-n3c(-c4cccnc4N)nc4cc(C)c(C)nc43)cc2)CC1 ; [2H]C([2H])([2H])NC1CCN(Cc2ccc(-n3c(-c4cccnc4N)nc4cc(C)c(C)nc43)cc2)CC1 ; [2H]C([2H])([2H])N(c1ccnc(C#N)n1)C1CCN(Cc2ccc(-n3c(-c4cccnc4N)nc4cc(C)c(C)nc43)cc2)CC1]

### Step 1: Tert-butyl (1-(4-(2-(2-aminopyridin-3-yl)-5,6-dimethyl-3H-imidazo[4,5-b]pyridin-3-yl)benzyl)piperidin-4-yl)(methyl-d4)carbamate

To a solution of Intermediate 138 (200 mg, 549 μmol) and tert-butyl (methyl-d₃)(piperidin-4-yl)carbamate (refer to Intermediate 41 for detail procedures, 143 mg, 660 μmol) in DMF (5 mL) were added K₂CO₃ (228 mg, 1.7 mmol) and NaI (82.4 mg, 550 μmol). The mixture was stirred at 50° C. for 8 hr. The mixture was concentrated under reduced pressure. After purified by silica gel flash chromatography (MeOH in CH₂Cl₂=0 to 15%), tert-butyl (1-(4-(2-(2-aminopyridin-3-yl)-5,6-dimethyl-3H-imidazo[4,5-b]pyridin-3-yl)benzyl)piperidin-4-yl)(methyl-d3)carbamate (165 mg, yield: 47%) was obtained as a yellow solid. MS: m/z=545.3 [M+H]⁺.

### Step 2: 3-(5,6-Dimethyl-3-(4-((4-((methyl-d₃)amino)piperidin-1-yl)methyl)phenyl)-3H-imidazo[4,5-b]pyridin-2-yl)pyridin-2-amine

To a solution of tert-butyl (1-(4-(2-(2-aminopyridin-3-yl)-5,6-dimethyl-3H-imidazo[4,5-b]pyridin-3-yl)benzyl)piperidin-4-yl)(methyl-d₃)carbamate (160 mg, 294 μmol) in CH₂Cl₂ (2 mL) was added TFA (1.54 g, 13.5 mmol). The mixture was stirred at 25° C. for 1 hr. The mixture was concentrated under reduced pressure, 3-(5,6-dimethyl-3-(4-((4-((methyl-d₃)amino)piperidin-1-yl)methyl)phenyl)-3H-imidazo[4,5-b]pyridin-2-yl)pyridin-2-amine (160 mg, crude) was obtained as a yellow solid. MS: m/z=445.1 [M+H]r.

### Step 3: 4-((1-(4-(2-(2-Aminopyridin-3-yl)-5,6-dimethyl-3H-imidazo[4,5-b]pyridin-3-yl)benzyl)piperidin-4-yl)(methyl-d₃)amino)pyrimidine-2-carbonitrile

4-Chloropyrimidine-2-carbonitrile (60.3 mg, 432 μmol), 3-(5,6-dimethyl-3-(4-((4-((methyl-d₃)amino)piperidin-1-yl)methyl)phenyl)-3H-imidazo[4,5-b]pyridin-2-yl)pyridin-2-amine (160 mg, 360 μmol) and DIEA (93 mg, 720 μmol) were taken up into a microwave tube in NMP (2 mL). The sealed tube was heated at 130° C. for 0.5 hr under microwave. The reaction mixture was diluted with H₂O (10 mL) and extracted with EtOAc (10 mL×2). The combined organic layers were washed with brine (30 mL), dried over Na₂SO₄, filtered and concentrated under reduced pressure. After purified by prep-HPLC (column: Waters xbridge 150×25 mm 10 m; mobile phase: [water (NH₄HCO₃)-ACN]; gradient: 38%-58% B over 11 min), 4-((1-(4-(2-(2-aminopyridin-3-yl)-5,6-dimethyl-3H-imidazo[4,5-b]pyridin-3-yl)benzyl)piperidin-4-yl)(methyl-d3)amino)pyrimidine-2-carbonitrile (Example 455, 13.5 mg, yield: 7% for two steps) was obtained as a yellow solid. MS: m/z=548.3 [M+H]⁺. 1H NMR (400 MHz, Methanol-d₄) δ 8.16 (d, J=6.4 Hz, 1H), 7.95 (dd, J=5.2, 1.6 Hz, 1H), 7.92-7.89 (m, 1H), 7.53 (d, J=8.4 Hz, 2H), 7.37 (d, J=8.4 Hz, 2H), 7.26 (dd, J=7.6, 1.6 Hz, 1H), 6.91-6.76 (m, 1H), 6.45 (dd, J=7.6, 5.2 Hz, 1H), 4.91-4.89 (m, 1H), 3.67 (s, 2H), 3.07 (d, J=11.2 Hz, 2H), 2.54 (s, 3H), 2.46 (s, 3H), 2.31-2.23 (m, 2H), 2.00-1.89 (m, 2H), 1.74-1.65 (m, 2H).

### Example 456: 4-((1-(4-(2-(2-Aminopyridin-3-yl)-5-(5-fluoropyridin-2-yl)-3H-imidazo[4,5-b]pyridin-3-yl)benzyl)piperidin-4-yl)amino)pyrimidine-2-carbonitrile

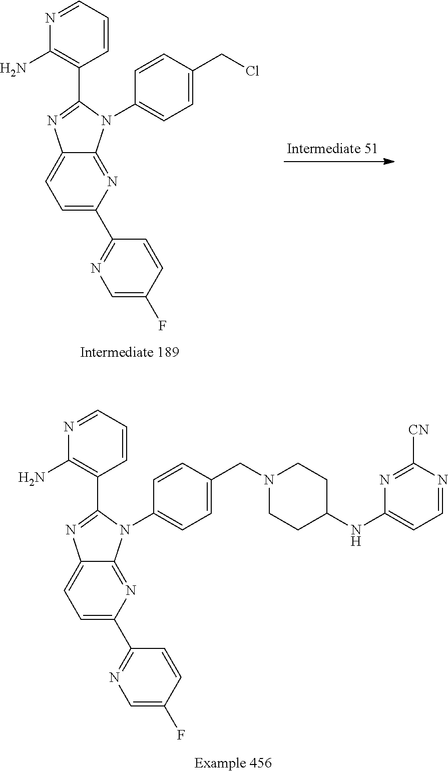 [SMILES: Nc1ncccc1-c1nc2ccc(-c3ccc(F)cn3)nc2n1-c1ccc(CCl)cc1 ; N#Cc1nccc(NC2CCN(Cc3ccc(-n4c(-c5cccnc5N)nc5ccc(-c6ccc(F)cn6)nc54)cc3)CC2)n1]

To a solution of Intermediate 189 (200 mg, 484 μmol) in DMF (5 mL) were added Intermediate 51 (113 mg, 557 μmol), NaI (35 mg, 232 μmol) and K₂CO₃ (256 mg, 1.86 mmol) at 25° C. The mixture was stirred at 50° C. for 3 hr. The reaction mixture was poured into H₂O (50 mL) and extracted with CH₂Cl₂ (20 mL×2). The combined organic layers were washed with brine (40 mL), dried over anhydrous Na₂SO₄, filtered, and concentrated to dryness. After purified by silica gel flash chromatography (Eluent of 0˜7% MeOH in CH₂Cl₂), 4-((1-(4-(2-(2-aminopyridin-3-yl)-5-(5-fluoropyridin-2-yl)-3H-imidazo[4,5-b]pyridin-3-yl)benzyl)piperidin-4-yl)amino)pyrimidine-2-carbonitrile (Example 456, 40.2 mg, yield: 14%) was obtained as an off white solid. MS: m/z=598.2 [M+H]y. ¹H NMR (400 MHz, Dimethylsulfoxide-d₆) δ 8.68 (d, J=2.8 Hz, 1H), 8.41 (d, J=8.4 Hz, 1H), 8.33 (d, J=8.4 Hz, 1H), 8.24 (dd, J=8.8, 4.8 Hz, 1H), 8.15-8.04 (m, 2H), 8.03-7.98 (m, 1H), 7.87-7.79 (m, 1H), 7.53-7.43 (m, 4H), 7.18 (dd, J=8.0, 2.0 Hz, 1H), 7.03 (br s, 2H), 6.69 (d, J=6.4 Hz, 1H), 6.39 (dd, J=7.6, 4.8 Hz, 1H), 3.91-3.74 (m, 1H), 3.61 (s, 2H), 2.89-2.78 (m, 2H), 2.24-2.13 (m, 2H), 1.97-1.84 (m, 2H), 1.60-1.44 (m, 2H).

### Example 457: 4-((1-(4-(2-(2-Aminopyridin-3-yl)-5-(methylamino)-3H-imidazo[4,5-b]pyridin-3-yl)benzyl)piperidin-4-yl)amino)pyrimidine-2-carbonitrile

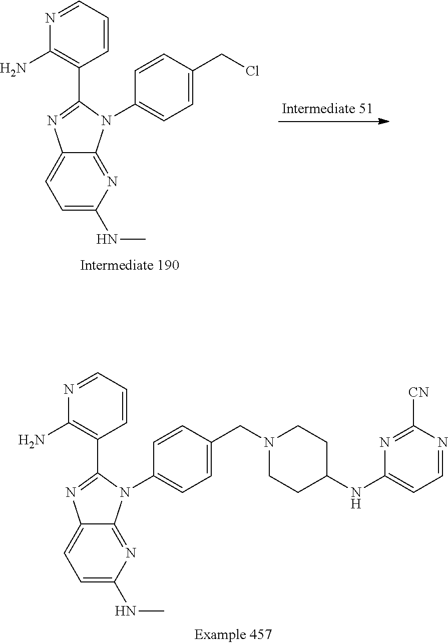 [SMILES: CNc1ccc2nc(-c3cccnc3N)n(-c3ccc(CCl)cc3)c2n1 ; CNc1ccc2nc(-c3cccnc3N)n(-c3ccc(CN4CCC(Nc5ccnc(C#N)n5)CC4)cc3)c2n1]

To a solution of Intermediate 190 (220 mg, 603 μmol) and Intermediate 51 (229 mg, 723 μmol) in DMF (10 mL) were added K₂CO₃ (416 mg, 3.02 mmol) and NaI (27.1 mg, 180 mol). The mixture was stirred at 50° C. for 1 hr. The reaction mixture was diluted with H₂O (30 mL) and extracted with EtOAc (30 mL×3). The combined organic layers were washed with H₂O (50 mL×3), dried over Na₂SO₄, filtered, and concentrated under reduced pressure. After purified by prep-HPLC (column: Waters xbridge 150*25 mm 10 μm; mobile phase: [water (NH₄HCO₃)-ACN]; gradient: 29%-59% B over 14 min), 4-((1-(4-(2-(2-aminopyridin-3-yl)-5-(methylamino)-3H-imidazo[4,5-b]pyridin-3-yl)benzyl)piperidin-4-yl)amino)pyrimidine-2-carbonitrile (Example 457, 120.6 mg, yield: 36%) was obtained as a yellow powder. MS: m/z=532.2 [M+H]⁺. ¹H NMR (400 MHz, Dimethylsulfoxide-d₆) δ 8.25-7.94 (m, 2H), 7.90 (dd, J=4.8, 1.6 Hz, 1H), 7.80 (d, J=8.4 Hz, 1H), 7.42 (d, J=8.4 Hz, 2H), 7.31 (d, J=8.4 Hz, 2H), 7.05 (br s, 2H), 6.93 (dd, J=8.0, 2.0 Hz, 1H), 6.84-6.52 (m, 2H), 6.48 (d, J=8.8 Hz, 1H), 6.28 (dd, J=8.0, 5.2 Hz, 1H), 3.93-3.65 (m, 1H), 3.56 (s, 2H), 2.85-2.75 (m, 2H), 2.71 (d, J=4.4 Hz, 3H), 2.20-2.10 (m, 2H), 1.93-1.80 (m, 2H), 1.54-1.41 (m, 2H).

### Example 458: 4-((1-(4-(2-(2-Aminopyridin-3-yl)-5-(bis(methyl-d₃)amino)-3H-imidazo[4,5-b]pyridin-3-yl)benzyl)piperidin-4-yl)amino)pyrimidine-2-carbonitrile

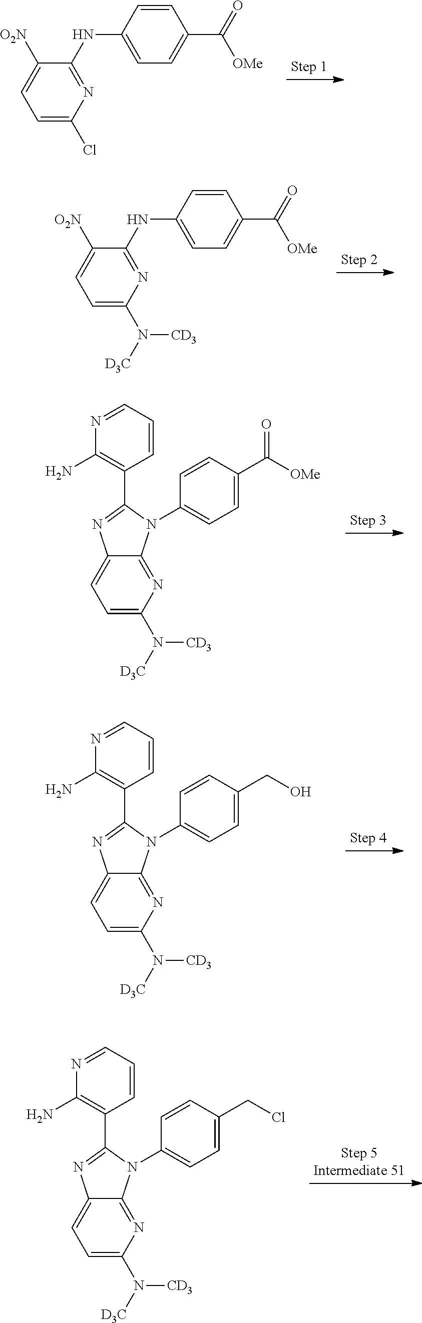

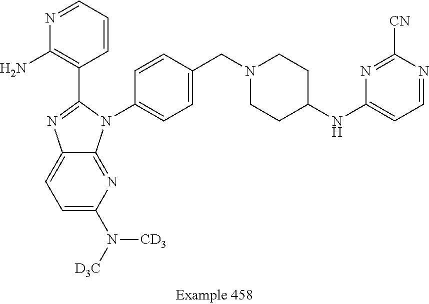 [SMILES: [2H]C([2H])([2H])N(c1ccc2nc(-c3cccnc3N)n(-c3ccc(CN4CCC(Nc5ccnc(C#N)n5)CC4)cc3)c2n1)C([2H])([2H])[2H]]

### Step 1: Methyl 4-((6-(bis(methyl-d₃)amino)-3-nitropyridin-2-yl)amino)benzoate

To a mixture of methyl 4-((6-chloro-3-nitropyridin-2-yl)amino)benzoate (refer to Intermediate 13 for detail procedures, 10.8 g, 35.2 mmol), bis(methyl-d₃)amine (2.0 g, 39.1 mmol) in ACN (120 mL) was added DIEA (15.1 g, 117 mmol). The mixture was degassed and purged with N₂ three times and stirred at 80° C. for 2 hr under N₂ atmosphere. The reaction mixture was filtered, methyl 4-((6-(bis(methyl-d₃)amino)-3-nitropyridin-2-yl)amino)benzoate (8.0 g, yield: 51%) was obtained as a yellow solid. MS: m/z=323.0 [M+H]⁺. ¹H NMR (400 MHz, Chloroform-d) δ 11.02 (s, 1H), 8.30 (d, J=9.6 Hz, 1H), 8.05-8.02 (m, 2H), 7.81-7.78 (m, 2H), 6.13 (d, J=9.6 Hz, 1H), 3.91 (s, 3H).

### Step 2: Methyl 4-(2-(2-aminopyridin-3-yl)-5-(bis(methyl-d3)amino)-3H-imidazo[4,5-b]pyridin-3-yl)benzoate

A mixture of methyl 4-((6-(bis(methyl-d₃)amino)-3-nitropyridin-2-yl)amino)benzoate (6.4 g, 19.8 mmol), 2-aminonicotinaldehyde (2.67 g, 21.8 mmol) and Na₂S₂O₄ (13.8 g, 79.4 mmol) in DMSO (150 mL) was degassed and purged with N₂ three times. The mixture was stirred at 100° C. for 16 hr under N₂ atmosphere. The reaction mixture was diluted with H₂O (200 mL) and extracted with EtOAc (200 mL×3). The combined organic layers were washed with brine (200 mL), dried over Na₂SO₄, filtered and concentrated under reduced pressure. After purified by silica gel flash chromatography (Eluent of 0˜29% EtOAc in petroleum ether), methyl 4-(2-(2-aminopyridin-3-yl)-5-(bis(methyl-d₃)amino)-3H-imidazo[4,5-b]pyridin-3-yl)benzoate (5.0 g, yield: 59%) was obtained as a yellow solid. MS: m/z=395.3 [M+H]+, ¹H NMR (400 MHz, Chloroform-d) δ 8.14 (d, J=8.8 Hz, 2H), 8.00 (dd, J=5.2, 2.0 Hz, 1H), 7.84 (d, J=8.8 Hz, 1H), 7.48 (d, J=8.4 Hz, 2H), 7.00-6.96 (m, 1H), 6.56 (d, J=8.8 Hz, 3H), 6.35 (dd, J=7.6, 5.2 Hz, 1H), 3.95 (s, 3H).

### Step 3: (4-(2-(2-Aminopyridin-3-yl)-5-(bis(methyl-d3)amino)-3H-imidazo[4,5-b]pyridin-3-yl)phenyl)methanol

To a mixture of methyl 4-(2-(2-aminopyridin-3-yl)-5-(bis(methyl-d)amino)-3H-imidazo[4,5-b]pyridin-3-yl)benzoate (5.0 g, 12.7 mmol) in THF (150 mL) was added LiAlH₄ (2.5 M in THF, 7.61 mL). The mixture was degassed and purged with N₂ three times and stirred at 0° C. for 2 hr under N₂ atmosphere. The reaction mixture was quenched with Na₂SO₄·10H₂O (1.5 g) at 0° C. and filtered. The filtrate was concentrated under reduced pressure, (4-(2-(2-aminopyridin-3-yl)-5-(bis(methyl-d3)amino)-3H-imidazo[4,5-b]pyridin-3-yl)phenyl)methanol (4.6 g, yield: 91%) was obtained as a yellow solid. MS: m/z=367.2 [M+H]⁺.

### Step 4: 2-(2-Aminopyridin-3-yl)-3-(4-(chloromethyl)phenyl)-N,N-bis(methyl-d3)-3H-imidazo[4,5-b]pyridin-5-amine

To a solution of (4-(2-(2-aminopyridin-3-yl)-5-(bis(methyl-d₃)amino)-3H-imidazo[4,5-b]pyridin-3-yl)phenyl)methanol (600 mg, 715 μmol) in CH₂Cl₂ (50 mL) was added SOCl₂ (8.96 g, 75.3 mmol). The mixture was stirred at 40° C. for 1 hr. The mixture was concentrated under reduced pressure, 2-(2-aminopyridin-3-yl)-3-(4-(chloromethyl)phenyl)-N,N-bis(methyl-d₃)-3H-imidazo[4,5-b]pyridin-5-amine (5.0 mg, yield: 97%) was obtained as a yellow solid. MS: m/z=385.0 [M+H]⁺.

### Step 5: 4-((1-(4-(2-(2-Aminopyridin-3-yl)-5-(bis(methyl-d₃)amino)-3H-imidazo[4,5-b]pyridin-3-yl)benzyl)piperidin-4-yl)amino)pyrimidine-2-carbonitrile

To a solution of 2-(2-aminopyridin-3-yl)-3-(4-(chloromethyl)phenyl)-N,N-bis(methyl-d₃)-3H-imidazo[4,5-b]pyridin-5-amine (300 mg, 779 μmol) and Intermediate 51 (158 mg, 779 μmol) in DMF (5 mL) were added NaI (58.4 mg, 390 μmol) and K₂CO₃ (539 mg, 3.90 mmol). The mixture was stirred at 50° C. for 1 hr. The reaction mixture was diluted with H₂O (20 mL) and extracted with EtOAc (50 mL×2). The combined organic layers were washed with brine (30 mL), dried over Na₂SO₄, filtered and concentrated under reduced pressure. After purified by silica gel flash chromatography (Eluent of 0˜6% MeOH in CH₂Cl₂), 4-((1-(4-(2-(2-aminopyridin-3-yl)-5-(bis(methyl-d3)amino)-3H-imidazo[4,5-b]pyridin-3-yl)benzyl)piperidin-4-yl)amino)pyrimidine-2-carbonitrile (Example 458, 89 mg, yield: 20%) was obtained as a yellow solid. MS: m/z=552.0 [M+H]⁺. ¹H NMR (400 MHz, Dimethylsulfoxide-d₆) δ 8.15 (dd, J=17.6, 6.0 Hz, 2H), 7.94-7.89 (m, 2H), 7.43 (d, J=8.0 Hz, 2H), 7.31 (d, J=8.4 Hz, 2H), 7.06 (s, 2H), 6.97 (dd, J=7.6, 1.6 Hz, 1H), 6.71-6.64 (m, 2H), 6.30 (dd, J=7.6, 4.8 Hz, 1H), 3.86-3.73 (m, 1H), 3.57 (s, 2H), 2.81 (d, J=10.4 Hz, 2H), 2.17-2.10 (m, 2H), 1.91-1.84 (m, 2H), 1.52-1.43 (m, 2H).

### Example 459: 4-((1-(4-(2-(2-Aminopyridin-3-yl)-5-((methyl-d₃)amino)-3H-imidazo[4,5-b]pyridin-3-yl)benzyl)piperidin-4-yl)amino)pyrimidine-2-carbonitrile

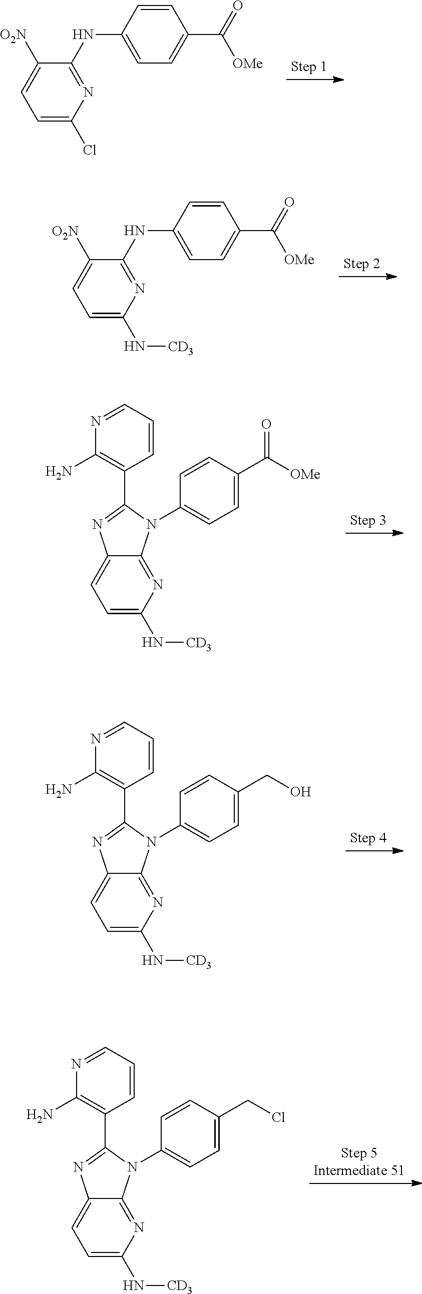 [SMILES: COC(=O)c1ccc(Nc2nc(Cl)ccc2[N+](=O)[O-])cc1 ; [2H]C([2H])([2H])Nc1ccc([N+](=O)[O-])c(Nc2ccc(C(=O)OC)cc2)n1 ; [2H]C([2H])([2H])Nc1ccc2nc(-c3cccnc3N)n(-c3ccc(C(=O)OC)cc3)c2n1 ; [2H]C([2H])([2H])Nc1ccc2nc(-c3cccnc3N)n(-c3ccc(CO)cc3)c2n1 ; [2H]C([2H])([2H])Nc1ccc2nc(-c3cccnc3N)n(-c3ccc(CCl)cc3)c2n1]

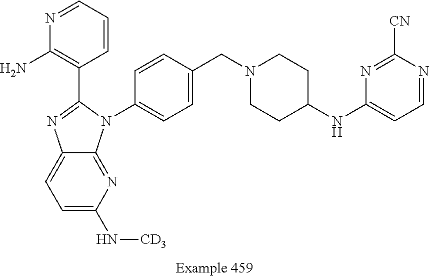 [SMILES: [2H]C([2H])([2H])Nc1ccc2nc(-c3cccnc3N)n(-c3ccc(CN4CCC(Nc5ccnc(C#N)n5)CC4)cc3)c2n1]

### Step 1: Methyl 4-((6-((methyl-d₃)amino)-3-nitropyridin-2-yl)amino)benzoate

To a mixture of methyl 4-((6-chloro-3-nitropyridin-2-yl)amino)benzoate (refer to Intermediate 13 for detail procedures, 2.5 g, 8.13 mmol) and CD₃NH₂·HCl (713 mg, 10.5 mmol) in CH₃CN (30 mL) was added DIEA (3.15 g, 24.3 mmol). The mixture was stirred at 90° C. for 2 hr. The reaction mixture was filtered and concentrated under reduced pressure. The crude product was triturated with EtOAc (50 mL) at 25° C. for 10 min, methyl 4-((6-((methyl-d4)amino)-3-nitropyridin-2-yl)amino)benzoate (2.3 g, yield. 76%) was obtained as a yellow solid. MS: m/z=305.9 [M+H]⁺. ¹H NMR (400 MHz, Dimethylsulfoxide-d₆) δ 11.06 (s, 1H), 8.43 (s, 1H), 8.09 (d, J=9.6 Hz, 1H), 7.98-7.92 (m, 4H), 6.19 (d, J=9.6 Hz, 1H), 3.83 (s, 3H).

### Step 2: Methyl 4-(2-(2-aminopyridin-3-yl)-5-((methyl-d₃)amino)-3H-imidazo[4,5-b]pyridin-3-yl)benzoate

To a mixture of methyl 4-((6-((methyl-d₃)amino)-3-nitropyridin-2-yl)amino)benzoate (2.30 g, 7.53 mmol), 2-aminonicotinaldehyde (1.10 g, 9.04 mmol) in DMSO (40 mL) was added Na₂S₂O₄ (5.25 g, 30.1 mmol). The mixture was stirred at 100° C. for 16 hr under N₂ atmosphere. The reaction mixture was diluted with H₂O (150 mL) and extracted with EtOAc (100 mL×3). The combined organic layers were washed with H₂O (100 mL×3), dried over Na₂SO₄, filtered and concentrated under reduced pressure to give a residue. After purified by column chromatography (eluent of 0-45% EtOAc in petroleum ether), methyl 4-(2-(2-aminopyridin-3-yl)-5-((methyl-d₃)amino)-3H-imidazo[4,5-b]pyridin-3-yl)benzoate (1.0 g, yield: 35%) was obtained as a yellow solid. MS: m/z=378.1 [M+H]⁺. ¹H NMR (400 MHz, Dimethylsulfoxide-d₆) δ 8.08-8.04 (m, 2H), 7.93 (dd, J=4.8, 1.6 Hz, 1H), 7.81 (d, J=8.8 Hz, 1H), 7.54-7.50 (m, 2H), 6.99 (dd, J=7.6, 2.0 Hz, 1H), 6.95-6.82 (m, 2H), 6.71 (s, 1H), 6.50 (d, J=8.8 Hz, 1H), 6.36 (dd, J=7.6, 4.8 Hz, 1H), 3.88 (s, 3H)

### Step 3: (4-(2-(2-Aminopyridin-3-yl)-5-((methyl-d₃)amino)-3H-imidazo[4,5-b]pyridin-3-yl)phenyl)methanol

To a mixture of methyl 4-(2-(2-aminopyridin-3-yl)-5-((methyl-d₃)amino)-3H-imidazo[4,5-b]pyridin-3-yl)benzoate (1 g, 2.65 mmol) in THF (50 mL) was added LiAlH₄ (2.5 M, 1.59 mL). The mixture was stirred at 25° C. for 1 hr under N₂ atmosphere. The reaction mixture was quenched with Na₂SO₄·10H₂O (2 g) at 0° C., filtered and concentrated under reduced pressure to give (4-(2-(2-aminopyridin-3-yl)-5-((methyl-d₃)amino)-3H-imidazo[4,5-b]pyridin-3-yl)phenyl)methanol (900 mg, yield: 81%) as a yellow solid. MS: m/z=350.3 [M+H]⁺.

### Step 4: 2-(2-Aminopyridin-3-yl)-3-(4-(chloromethyl)phenyl)-N-(methyl-d₃)-3H-imidazo[4,5-b]pyridin-5-amine

To a mixture of (4-(2-(2-aminopyridin-3-yl)-5-((methyl-d₃)amino)-3H-imidazo[4,5-b]pyridin-3-yl)phenyl)methanol (900 mg, 2.58 mmol) in CH₂Cl₂ (5 mL) was added SOCl₂ (3.28 g, 27.5 mmol). The mixture was stirred at 40° C. for 1 hr. The reaction mixture was concentrated under reduced pressure to give 2-(2-aminopyridin-3-yl)-3-(4-(chloromethyl)phenyl)-N-(methyl-d)-3H-imidazo[4,5-b]pyridin-5-amine (950 mg, yield: 98%) as a brown solid. MS: m/z=368.1 [M+H]⁺.

### Step 5: 4-((1-(4-(2-(2-Aminopyridin-3-yl)-5-((methyl-d₃)amino)-3H-imidazo[4,5-b]pyridin-3-yl)benzyl)piperidin-4-yl)amino)pyrimidine-2-carbonitrile

To a mixture of 2-(2-aminopyridin-3-yl)-3-(4-(chloromethyl)phenyl)-N-(methyl-d)-3H-imidazo[4,5-b]pyridin-5-amine (300 mg, 815 μmol) and Intermediate 51 (200 mg, 984 μmol) in DMF (10 mL) were added K₂CO₃ (563 mg, 4.08 mmol) and NaI (36.6 mg, 244 μmol). The mixture was stirred at 50° C. for 1 hr. The reaction mixture was diluted with H₂O (50 mL) and extracted with EtOAc (50 mL×3). The combined organic layers were washed with H₂O (50 mL×3), dried over Na₂SO₄, filtered and concentrated under reduced pressure to give a residue. After purified by column chromatography (eluent of 0-8% MeOH in CH₂Cl₂), 4-((1-(4-(2-(2-aminopyridin-3-yl)-5-((methyl-d₃)amino)-3H-imidazo[4,5-b]pyridin-3-yl)benzyl)piperidin-4-yl)amino)pyrimidine-2-carbonitrile (Example 459, 123.1 mg, yield: 28%) was obtained as a yellow solid. MS: m/z=535.1 [M+H]⁺. ¹H NMR (400 MHz, Dimethylsulfoxide-d₆) δ 8.15-7.97 (m, 2H), 7.93-7.85 (m, 1H), 7.80 (d, J=8.8 Hz, 1H), 7.42 (d, J=7.2 Hz, 2H), 7.30 (d, J=7.6 Hz, 2H), 7.05 (br s, 2H), 6.93 (d, J=7.2 Hz, 1H), 6.72-6.60 (m, 2H), 6.47 (d, J=8.4 Hz, 1H), 6.32-6.24 (m, 1H), 3.85-3.75 (m, 1H), 3.56 (s, 2H), 2.85-2.75 (m, 2H), 2.20-2.10 (m, 2H), 1.94-1.81 (m, 2H), 1.52-1.40 (m, 2H).

### Example 460: 4-((1-(4-(2-(2-Aminopyridin-3-yl)-5-(azetidin-1-yl)-3H-imidazo[4,5-b]pyridin-3-yl)benzyl)piperidin-4-yl)amino)pyrimidine-2-carbonitrile

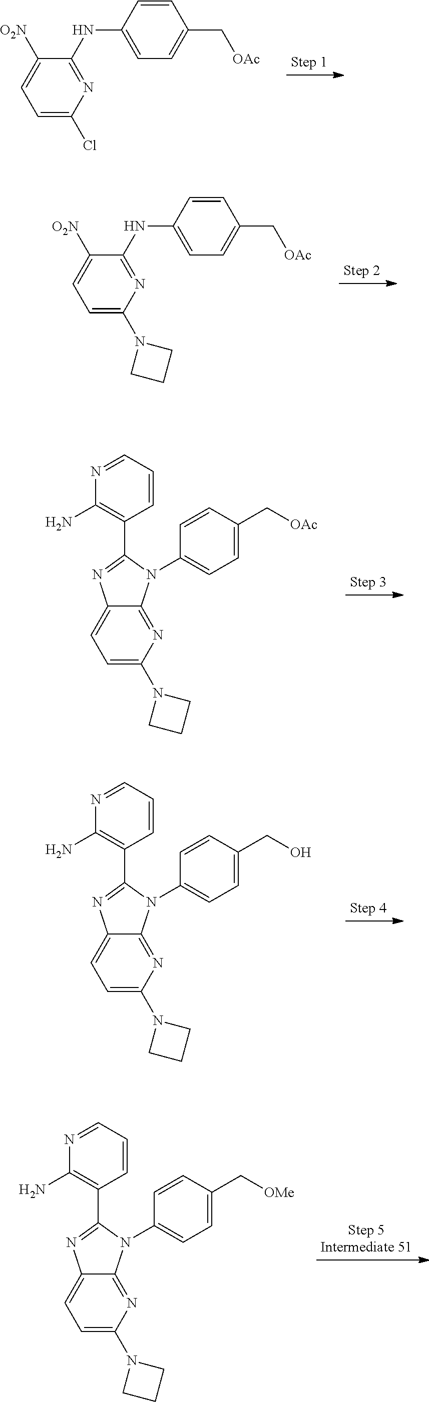 [SMILES: CC(=O)OCc1ccc(Nc2nc(Cl)ccc2[N+](=O)[O-])cc1 ; CC(=O)OCc1ccc(Nc2nc(N3CCC3)ccc2[N+](=O)[O-])cc1 ; CC(=O)OCc1ccc(-n2c(-c3cccnc3N)nc3ccc(N4CCC4)nc32)cc1 ; Nc1ncccc1-c1nc2ccc(N3CCC3)nc2n1-c1ccc(CO)cc1 ; COCc1ccc(-n2c(-c3cccnc3N)nc3ccc(N4CCC4)nc32)cc1]

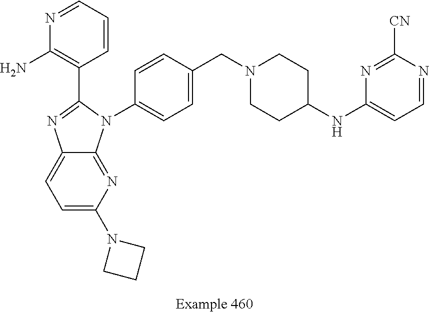 [SMILES: N#Cc1nccc(NC2CCN(Cc3ccc(-n4c(-c5cccnc5N)nc5ccc(N6CCC6)nc54)cc3)CC2)n1]

### Step 1: 4-((6-(Azetidin-1-yl)-3-nitropyridin-2-yl)amino)benzyl acetate

To a solution of 4-((6-chloro-3-nitropyridin-2-yl)amino)benzyl acetate (refer to Intermediate 178 for detail procedures, 5 g, 15.5 mmol) in MeCN (100 mL) were added DIEA (5.02 g, 38.9 mmol) and azetidine (2.18 g, 23.3 mmol, HCl salt). The mixture was stirred at 90° C. for 16 hr. The reaction was concentrated under reduced pressure. After purified by silica gel flash chromatography (Eluent of 50%˜100% EtOAc in petroleum ether), 4-((6-(azetidin-1-yl)-3-nitropyridin-2-yl)amino)benzyl acetate (5.0 g, yield: 92%) was obtained as a yellow solid. MS: m/z=342.9 [M+H]⁺. ¹H NMR (400 MHz, Dimethylsulfoxide-d₆) δ 10.84 (s, 1H), 8.16 (d, J=9.2 Hz, 1H), 7.80 (d, J=8.4 Hz, 2H), 7.34 (d, J=8.4 Hz, 2H), 5.97 (d, J=9.2 Hz, 1H), 5.04 (s, 2H), 4.17 (t, J=7.2 Hz, 4H), 2.41-2.33 (m, 2H), 2.06 (s, 3H).

### Step 2: 4-(2-(2-Aminopyridin-3-yl)-5-(azetidin-1-yl)-3H-imidazo[4,5-b]pyridin-3-yl)benzyl acetate

To a solution of 4-((6-(azetidin-1-yl)-3-nitropyridin-2-yl)amino)benzyl acetate (500 mg, 1.46 mmol) in DMSO (20 mL) were added Na₂S₂O₄ (1.17 g, 5.84 mmol, 87% purity) and 2-aminonicotinaldehyde (214 mg, 1.75 mmol). The mixture was stirred at 100° C. for 4 hr. The reaction mixture was quenched with H₂O (25 mL) at 25° C., then diluted with CH₂Cl₂ (50 mL) and washed with H₂O (50 mL×3) and brine (100 mL), dried over Na₂SO₄, filtered and concentrated under reduced pressure. After purified by silica gel flash chromatography (Eluent of 30%˜60% EtOAc in petroleum ether), 4-(2-(2-aminopyridin-3-yl)-5-(azetidin-1-yl)-3H-imidazo[4,5-b]pyridin-3-yl)benzyl acetate (280 mg, yield: 46%) was obtained as a green solid. MS: m/z=415.4 [M+H]⁺. ¹H NMR (400 MHz, Dimethylsulfoxide-d₆) δ7.96-7.88 (m, 2H), 7.48 (d, J=8.4 Hz, 2H), 7.36 (d, J=8.4 Hz, 2H), 7.01 (dd, J=7.6, 1.6 Hz, 1H), 6.90 (br s, 2H), 6.41-6.31 (m, 2H), 5.15 (s, 2H), 3.91 (t, J=7.2 Hz, 4H), 2.33-2.23 (m, 2H), 2.10 (s, 3H).

### Step 3: (4-(2-(2-Aminopyridin-3-yl)-5-(azetidin-1-yl)-3H-imidazo[4,5-b]pyridin-3-yl)phenyl)methanol

To a solution of 4-(2-(2-aminopyridin-3-yl)-5-(azetidin-1-yl)-3H-imidazo[4,5-b]pyridin-3-yl)benzyl acetate (280 mg, 676 μmol) in THF (4 mL), MeOH (4 mL) and H₂O (1 mL) was added K₂CO₃ (280 mg, 2.03 mmol). The mixture was stirred at 25° C. for 0.5 hr. The reaction mixture was quenched with H₂O (5 mL) at 25° C., and extracted with CH₂Cl₂ (20 mL×3). The combined organic layers were washed with brine (50 mL), dried over Na₂SO₄, filtered, and concentrated under reduced pressure to give (4-(2-(2-aminopyridin-3-yl)-5-(azetidin-1-yl)-3H-imidazo[4,5-b]pyridin-3-yl)phenyl)methanol (240 mg, yield: 95%) as a yellow solid. MS: m/z=373.1 [M+H]⁺. ¹H NMR (400 MHz, Dimethylsulfoxide-d₆) δ 7.96-7.87 (m, 2H), 7.43 (d, J=8.0 Hz, 2H), 7.30 (d, J=8.0 Hz, 2H), 7.00-6.97 (m, 3H), 6.37 (d, J=8.8 Hz, 1H), 6.33 (dd, J=7.6, 4.8 Hz, 1H), 5.34 (t, J=6.0 Hz, 1H), 4.57 (d, J=6.0 Hz, 2H), 3.90 (t, J=7.2 Hz, 4H), 2.32-2.22 (m, 2H).

### Step 4: 4-(2-(2-Aminopyridin-3-yl)-5-(azetidin-1-yl)-3H-imidazo[4,5-b]pyridin-3-yl)benzyl methanesulfonate

To a solution of (4-(2-(2-aminopyridin-3-yl)-5-(azetidin-1-yl)-3H-imidazo[4,5-b]pyridin-3-yl)phenyl)methanol (140 mg, 376 μmol) in CH₂Cl₂ (15 mL) were added TEA (114 mg, 1.13 mmol) and MsCl (140 mg, 1.22 mmol) at 0° C. The mixture was stirred at 0° C. for 1 hr. Additional MsCl (100 mg, 873 μmol) was added at 0° C. The mixture was stirred at 0° C. for 15 minutes. The reaction mixture was quenched with saturated NaHCO₃ aqueous (20 mL) at 0° C. and was then diluted with CH₂Cl₂ (10 mL) and extracted with CH₂Cl₂ (15 mL×2). The combined organic layers were washed with brine (50 mL), dried over Na₂SO₄, filtered and concentrated under reduced pressure. 4-(2-(2-Aminopyridin-3-yl)-5-(azetidin-1-yl)-3H-imidazo[4,5-b]pyridin-3-yl)benzyl methanesulfonate (170 mg) was obtained as a yellow solid. MS: m/z=451.0 [M+H]⁺.

### Step 5: 4-((1-(4-(2-(2-Aminopyridin-3-yl)-5-(azetidin-1-yl)-3H-imidazo[4,5-b]pyridin-3-yl)benzyl)piperidin-4-yl)amino)pyrimidine-2-carbonitrile

To a solution of 4-(2-(2-aminopyridin-3-yl)-5-(azetidin-1-yl)-3H-imidazo[4,5-b]pyridin-3-yl)benzyl methanesulfonate (170 mg, 377 μmol) and Intermediate 51 (120 mg, 377 μmol, TFA) in DMF (2 mL) were added K₂CO₃ (209 mg, 1.51 mmol) and NaI (11.3 mg, 75.5 μmol). The mixture was stirred at 50° C. for 1 hr. The mixture was filtered through Celite and concentrated under reduced pressure. After purified by prep-HPLC(column: Waters xbridge 150×25 mm×10 μm; mobile phase: [water (NH₃H₂O+NH₄HCO₃)-ACN]; gradient: 30%-60% B over 11 min), 4-((1-(4-(2-(2-Aminopyridin-3-yl)-5-(azetidin-1-yl)-3H-imidazo[4,5-b]pyridin-3-yl)benzyl)piperidin-4-yl)amino)pyrimidine-2-carbonitrile (Example 460, 17.7 mg, yield: 8.4% for two steps) was obtained as a yellow solid. MS: m/z=558.5 [M+H]⁺. ¹H NMR (400 MHz, Dimethylsulfoxide-d₆) δ 8.08-8.05 (d, J=6.8 Hz, 2H), 7.94-7.89 (m, 2H), 7.42 (d, J=8.4 Hz, 2H), 7.30 (d, J=8.4 Hz, 2H), 7.01 (br s, 2H), 6.94 (d, J=6.8 Hz, 1H), 6.67 (d, J=6.4 Hz, 1H), 6.37 (d, J=8.4 Hz, 1H), 6.29 (dd, J=7.6, 4.8 Hz, 1H), 3.91 (t, 1=7.6 Hz, 4H), 3.84-3.75 (m, 1H), 3.56 (s, 2H), 2.85-2.77 (m, 2H), 2.32-2.24 (m, 2H), 2.19-2.09 (m, 2H), 1.94-1.82 (m, 2H), 1.53-1.43 (m, 2H).

### Example 461: 4-((1-(4-(2-(2-Aminopyridin-3-yl)-5-(isoxazol-4-yl)-3H-imidazo[4,5-b]pyridin-3-yl)benzyl)piperidin-4-yl)amino)pyrimidine-2-carbonitrile

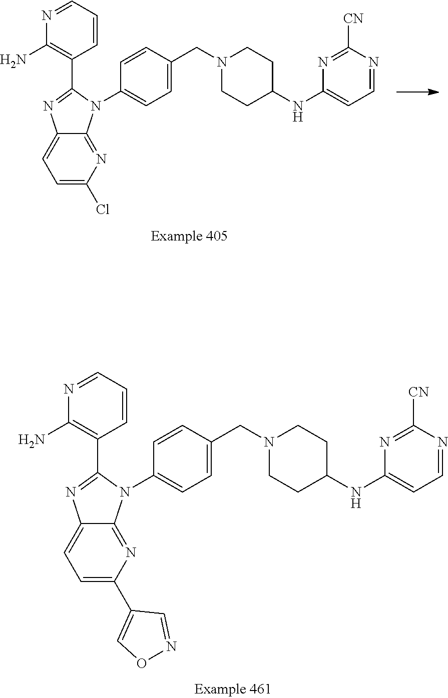 [SMILES: N#Cc1nccc(NC2CCN(Cc3ccc(-n4c(-c5cccnc5N)nc5ccc(Cl)nc54)cc3)CC2)n1 ; N#Cc1nccc(NC2CCN(Cc3ccc(-n4c(-c5cccnc5N)nc5ccc(-c6cnoc6)nc54)cc3)CC2)n1]

A mixture of Example 405 (50 mg, 93.1 μmol), isoxazol-4-ylboronic acid (26.3 mg, 233 μmol), cata CXium A PdG₃ (67.8 mg, 93.1 μmol), K₃PO₄ (59.3 mg, 279 μmol) and PCy₃ (13.1 mg, 46.6 μmol) in DMF (2 mL) was degassed and purged with N₂ three times, and then was stirred at 80° C. for 3 hr under N₂ atmosphere. The reaction mixture was filtered and concentrated. After purified by prep-HPLC (column: Waters xbridge 150×25 mm 10 μm; mobile phase: [water (NH₄HCO₃)-ACN]; gradient: 40%-70% B over 11 min), 4-((1-(4-(2-(2-aminopyridin-3-yl)-5-(isoxazol-4-yl)-3H-imidazo[4,5-b]pyridin-3-yl)benzyl)piperidin-4-yl)amino)pyrimidine-2-carbonitrile (Example 461, 19.0 mg, yield: 35%) was obtained as a light-yellow lyophilized powder. MS: m/z=570.3 [M+H]⁺. ¹H NMR (400 MHz, Dimethylsulfoxide-d₆) δ 8.14-8.09 (m, 2H), 8.01-7.94 (m, 2H), 7.61-7.38 (m, 6H), 7.32-7.14 (m, 1H), 7.12-7.06 (m, 1H), 7.01 (d, J=6.4 Hz, 2H), 6.68 (d, J=5.6 Hz, 1H), 6.39-6.34 (m, 1H), 4.06-3.77 (m, 3H), 3.09-2.98 (m, 2H), 2.61-2.55 (m, 2H), 2.02-1.94 (m, 2H), 1.63-1.54 (m, 2H).

### Example 462: 4-((1-(4-(2-(2-Aminopyridin-3-yl)-5-(oxazol-5-yl)-3H-imidazo[4,5-b]pyridin-3-yl)benzyl)piperidin-4-yl)amino)pyrimidine-2-carbonitrile

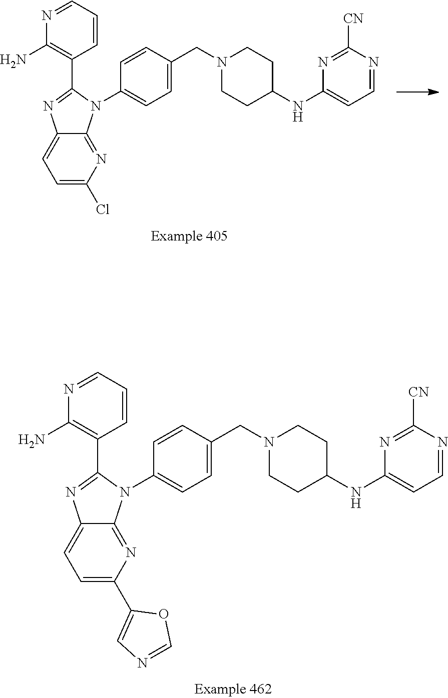 [SMILES: N#Cc1nccc(NC2CCN(Cc3ccc(-n4c(-c5cccnc5N)nc5ccc(Cl)nc54)cc3)CC2)n1 ; N#Cc1nccc(NC2CCN(Cc3ccc(-n4c(-c5cccnc5N)nc5ccc(-c6cnco6)nc54)cc3)CC2)n1]

A mixture of Example 405 (200 mg, 372 μmol), 5-(4,4,5,5-tetramethyl-1,3,2-dioxaborolan-2-yl)-2-(triisopropylsilyl)oxazole (196 mg, 559 μmol), cata CXium A PdG₃ (271 mg, 372 mol), K₃PO₄ (52.2 mg, 186 μmol) and PCy. (237 mg, 1.12 mmol) in DMF (1 mL) was degassed and purged with N₂ three times, and then was stirred at 80° C. for 3 hr under N₂ atmosphere. The reaction mixture was filtered and concentrated. The crude product was purified by prep-HPLC (column: Phenomenex C18 150×25 mm×10 μm; mobile phase: [water (NH₄HCO₃)-ACN]; gradient: 30%-60% B over 10 min). 4-((1-(4-(2-(2-Aminopyridin-3-yl)-5-(oxazol-5-yl)-3H-imidazo[4,5-b]pyridin-3-yl)benzyl)piperidin-4-yl)amino)pyrimidine-2-carbonitrile (Example 462, 14.1 mg, yield: 6.6%) was obtained as a light-yellow lyophilized powder. MS: m/z=570.2 [M+H]⁺. ¹H NMR (400 MHz, Methanol-d₄) δ 8.29 (s, 1H), 8.24 (d, J=8.4 Hz, 1H), 8.03-7.97 (m, 2H), 7.86 (d, J=8.4 Hz, 1H), 7.60 (s, 1H), 7.55 (d, J=8.4 Hz, 2H), 7.43 (d, J=8.4 Hz, 2H), 7.31 (dd, J=7.6, 1.6 Hz, 1H), 6.60 (d, J=5.6 Hz, 1H), 6.47 (dd, J=7.6, 5.2 Hz, 1H), 4.06-3.87 (m, 1H), 3.67 (s, 2H), 2.98 (d, J=14.0 Hz, 2H), 2.33-2.25 (m, 2H), 2.02 (d, J=11.2 Hz, 2H), 1.67-1.57 (m, 2H).

### Example 463: 4-((1-(4-(2-(2-Aminopyridin-3-yl)-5-(5-methylisoxazol-4-yl)-3H-imidazo[4,5-b]pyridin-3-yl)benzyl)piperidin-4-yl)amino)pyrimidine-2-carbonitrile

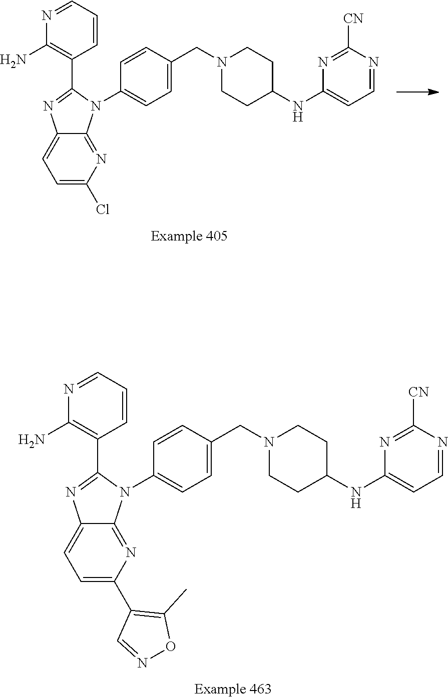 [SMILES: N#Cc1nccc(NC2CCN(Cc3ccc(-n4c(-c5cccnc5N)nc5ccc(Cl)nc54)cc3)CC2)n1 ; Cc1oncc1-c1ccc2nc(-c3cccnc3N)n(-c3ccc(CN4CCC(Nc5ccnc(C#N)n5)CC4)cc3)c2n1]

A mixture of Example 405 (180 mg, 335 μmol), 5-methyl-4-(4,4,5,5-tetramethyl-1,3,2-dioxaborolan-2-yl)isoxazole (140 mg, 670 μmol), cataCXiumAPdG3 (122 mg, 168 μmol), K₃PO₄ (142 mg, 670 μmol) and PCy₃ (9.40 mg, 34 μmol) in DMF (2 mL) was degassed and purged with N₂ three times. The mixture was stirred at 120° C. for 16 hr under N₂ atmosphere. The reaction mixture was poured into H₂O (15 mL), extracted with EtOAc (15 mL×3). The organic phase was dried over anhydrous Na₂SO₄, filtered and concentrated under reduced pressure. After purified by prep-HPLC (column: Waters xbridge 150×25 mm 10 μm; mobile phase: [water (NH₄HCO₃)-ACN]; gradient: 32%-62% B over 11 min), 4-((1-(4-(2-(2-aminopyridin-3-yl)-5-(5-methylisoxazol-4-yl)-3H-imidazo[4,5-b]pyridin-3-yl)benzyl)piperidin-4-yl)amino)pyrimidine-2-carbonitrile (Example 463, 20.8 mg, yield: 11%) was obtained as an off white lyophilized powder. MS: m/z=584.3 [M+H]⁺. ¹H NMR (400 MHz, Methanol-d₄) δ 8.29 (d, J=8.8 Hz, 1H), 8.11-7.87 (m, 2H), 7.59 (d, J=8.4 Hz, 2H), 7.50 (d, J=8.4 Hz, 1H), 7.42 (d, J=8.4 Hz, 2H), 7.36 (dd, J=8.0, 2.0 Hz, 1H), 6.68-6.52 (m, 1H), 6.46 (dd, J=8.0, 5.2 Hz, 1H), 4.95-4.89 (m, 1H), 4.05-3.80 (m, 1H), 3.69 (s, 2H), 3.05-2.86 (m, 2H), 2.30 (s, 3H), 2.30-2.23 (m, 2H), 2.06-1.97 (m, 2H), 1.68-1.56 (m, 2H).

### Example 464: 4-((1-(4-(2-(2-Aminopyridin-3-yl)-5-(3-methylisoxazol-4-yl)-3H-imidazo[4,5-b]pyridin-3-yl)benzyl)piperidin-4-yl)amino)pyrimidine-2-carbonitrile

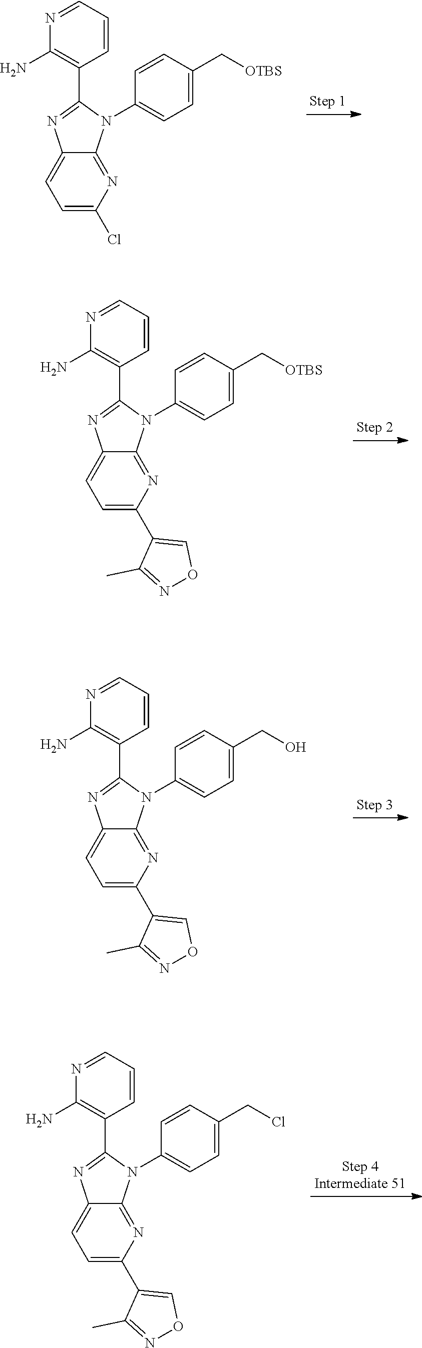 [SMILES: CC(C)(C)[Si](C)(C)OCc1ccc(-n2c(-c3cccnc3N)nc3ccc(Cl)nc32)cc1 ; Cc1nocc1-c1ccc2nc(-c3cccnc3N)n(-c3ccc(CO[Si](C)(C)C(C)(C)C)cc3)c2n1 ; Cc1nocc1-c1ccc2nc(-c3cccnc3N)n(-c3ccc(CO)cc3)c2n1 ; Cc1nocc1-c1ccc2nc(-c3cccnc3N)n(-c3ccc(CCl)cc3)c2n1]

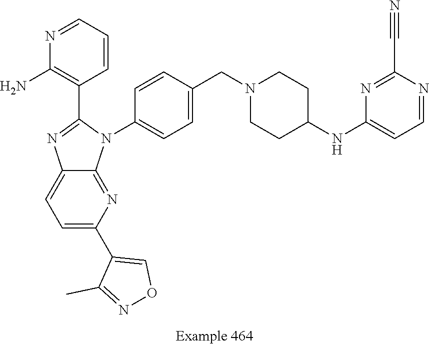 [SMILES: Cc1nocc1-c1ccc2nc(-c3cccnc3N)n(-c3ccc(CN4CCC(Nc5ccnc(C#N)n5)CC4)cc3)c2n1]

### Step 1: 3-(3-(4-(((Tert-butyldimethylsilyl)oxy)methyl)phenyl)-5-(3-methylisoxazol-4-yl)-3H-imidazo[4,5-b]pyridin-2-yl)pyridin-2-amine

To a solution of 3-(3-(4-(((tert-butyldimethylsilyl)oxy)methyl)phenyl)-5-chloro-3H-imidazo[4,5-b]pyridin-2-yl)pyridin-2-amine (refer to Intermediate 117 for detail procedures, 500 mg, 1.07 mmol) and (3-methylisoxazol-4-yl)boronic acid (150 mg, 1.18 mmol) in 1,4-dioxane (10 mL) and H₂O (2 mL) were added Pd(dppf)Cl₂ (78.5 mg, 107 μmol) and Cs₂CO₃ (1.05 g, 3.22 mmol). The mixture was degassed and purged with N₂ three times, and then was stirred at 100° C. for 16 hr under N₂ atmosphere. The reaction mixture was diluted with H₂O (100 mL) and extracted with CH₂Cl₂ (100 mL×2). The combined organic layers were washed with brine (100 mL), dried over Na₂SO₄, filtered, and concentrated under reduced pressure. After purified by silica gel flash chromatography (Eluent of 20%˜50% EtOAc in petroleum ether), 3-(3-(4-(((tert-butyldimethylsilyl)oxy)methyl)phenyl)-5-(3-methylisoxazol-4-yl)-3H-imidazo[4,5-b]pyridin-2-yl)pyridin-2-amine (180 mg, yield: 30%) was obtained as a brown solid. MS: m/z=513.1 [M+H]⁺. ¹H NMR (400 MHz, Dimethylsulfoxide-d₆) δ 9.47 (s, 1H), 8.27 (d, J=8.4 Hz, 1H), 8.02-7.98 (m, 1H), 7.78 (d, J=8.4 Hz, 1H), 7.50-7.44 (m, 4H), 7.21 (dd, J=7.6, 1.6 Hz, 1H), 7.04 (s, 2H), 6.40 (dd, J=7.6, 4.8 Hz, 1H), 4.81 (s, 2H), 2.38 (s, 3H), 0.91 (s, 9H), 0.09 (s, 6H).

### Step 2: (4-(2-(2-Aminopyridin-3-yl)-5-(3-methylisoxazol-4-yl)-3H-imidazo[4,5-b]pyridin-3-yl)phenyl)methanol

To a solution of 3-(3-(4-(((tert-butyldimethylsilyl)oxy)methyl)phenyl)-5-(3-methylisoxazol-4-yl)-3H-imidazo[4,5-b]pyridin-2-yl)pyridin-2-amine (180 mg, 351 μmol) in THF (5 mL) was added TBAF (1 M, 0.7 mL). The mixture was stirred at 25° C. for 0.5 hr. The reaction mixture was diluted with H₂O (20 mL) and extracted with CH₂Cl₂ (20 mL×2). The combined organic layers were washed with brine (20 mL×2), dried over Na₂SO₄, filtered, and concentrated under reduced pressure. (4-(2-(2-Aminopyridin-3-yl)-5-(3-methylisoxazol-4-yl)-3H-imidazo[4,5-b]pyridin-3-yl)phenyl)methanol (110 mg, yield: 79%) was obtained as a yellow solid. MS: m/z=398.9 [M+H]⁺. ¹H NMR (400 MHz, Dimethylsulfoxide-d₆) δ 9.45 (s, 1H), 8.25 (d, J=8.4 Hz, 1H), 8.00 (dd, J=4.8, 2.0 Hz, 1H), 7.76 (d, J=8.4 Hz, 1H), 7.48-7.41 (m, 4H), 7.21 (dd, J=7.6, 2.0 Hz, 1H), 6.98 (s, 2H), 6.44-6.39 (m, 1H), 5.34 (t, J=5.6 Hz, 1H), 4.58 (d, J=5.2 Hz, 2H), 2.39 (s, 3H).

### Step 3: 3-(3-(4-(Chloromethyl)phenyl)-5-(3-methylisoxazol-4-yl)-3H-imidazo[4,5-b]pyridin-2-yl)pyridin-2-amine

To a solution of (4-(2-(2-aminopyridin-3-yl)-5-(3-methylisoxazol-4-yl)-3H-imidazo[4,5-b]pyridin-3-yl)phenyl)methanol (110 mg, 276 μmol) in CH₂Cl₂ (2 mL) was added SOCl₂ (98.5 mg, 828 μmol). The mixture was stirred at 40° C. for 0.5 hr. The reaction was concentrated under reduced pressure to give 3-(3-(4-(chloromethyl)phenyl)-5-(3-methylisoxazol-4-yl)-3H-imidazo[4,5-b]pyridin-2-yl)pyridin-2-amine (130 mg, crude) as a yellow solid and used directly in the next step without purification. MS: m/z=417.0 [M+H]⁺.

### Step 4: 4-((1-(4-(2-(2-Aminopyridin-3-yl)-5-(3-methylisoxazol-4-yl)-3H-imidazo[4,5-b]pyridin-3-yl)benzyl)piperidin-4-yl)amino)pyrimidine-2-carbonitrile

To a solution of 3-(3-(4-(chloromethyl)phenyl)-5-(3-methylisoxazol-4-yl)-3H-imidazo[4,5-b]pyridin-2-yl)pyridin-2-amine (130 mg, 312 μmol) and Intermediate 51 (98.9 mg, 312 μmol, TFA salt) in DMF (2 mL) were added K₂CO₃ (216 mg, 1.56 mmol) and NaI (4.67 mg, 31.2 μmol). The mixture was stirred at 60° C. for 1 hr. After purified by prep-HPLC (column: Waters xbridge 150×25 mm×10 μm; mobile phase: [water (NH₄HCO₃)-ACN]; gradient: 35%-65% B over 10 min), 4-((1-(4-(2-(2-aminopyridin-3-yl)-5-(3-methylisoxazol-4-yl)-3H-imidazo[4,5-b]pyridin-3-yl)benzyl)piperidin-4-yl)amino)pyrimidine-2-carbonitrile (Example 464, 40.7 mg, yield: 22% for two steps) was obtained as a yellow solid. MS: m/z=584.4 [M+H]⁺. ¹H NMR (400 MHz, Dimethylsulfoxide-d₆) δ 9.47 (s, 1H), 8.26 (d, J=8.4 Hz, 1H), 8.10-7.98 (m, 3H), 7.77 (d, J=8.4 Hz, 1H), 7.48-7.41 (m, 4H), 7.22 (dd, J=7.6, 1.6 Hz, 1H), 7.08 (s, 2H), 6.66 (d, J=6.0 Hz, 1H), 6.43-6.37 (m, 1H), 3.91-3.72 (m, 1H), 3.57 (s, 2H), 2.82-2.76 (m, 2H), 2.34 (s, 3H), 2.14-2.08 (m, 2H), 1.90-1.83 (m, 2H), 1.51-1.42 (m, 2H).

### Example 465: 4-((1-(4-(2-(2-Aminopyridin-3-yl)-5-(3-methyl-1H-pyrazol-5-yl)-3H-imidazo[4,5-b]pyridin-3-yl)benzyl)piperidin-4-yl)amino)pyrimidine-2-carbonitrile

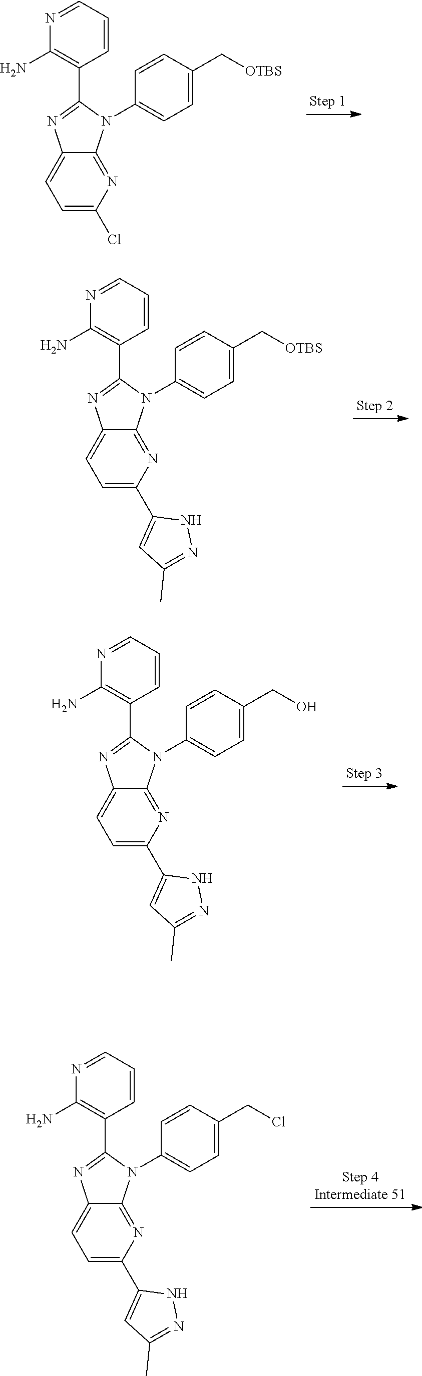

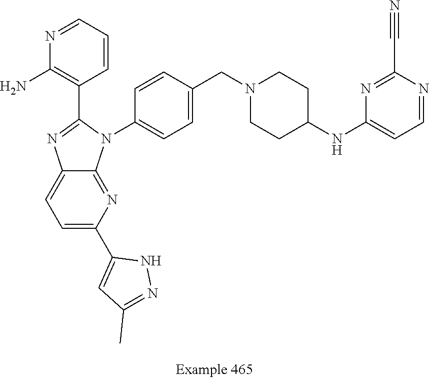 [SMILES: Cc1cc(-c2ccc3nc(-c4cccnc4N)n(-c4ccc(CN5CCC(Nc6ccnc(C#N)n6)CC5)cc4)c3n2)[nH]n1]

### Step 1: 3-(3-(4-(((Tert-butyldimethylsilyl)oxy)methyl)phenyl)-5-(3-methyl-1H-pyrazol-5-yl)-3H-imidazo[4,5-b]pyridin-2-yl)pyridin-2-amine

To a solution of 3-(3-(4-(((tert-butyldimethylsilyl)oxy)methyl)phenyl)-5-chloro-3H-imidazo[4,5-b]pyridin-2-yl)pyridin-2-amine (refer to Intermediate 117 for detail procedures, 500 mg, 1.07 mmol) and (3-methyl-1H-pyrazol-5-yl)boronic acid (149 mg, 1.18 mmol) in 1,4-dioxane (10 mL) and H₂O (2 mL) were added Pd(dppf)Cl₂ (78.5 mg, 107 μmol) and Cs₂CO₃ (1.05 g, 3.22 mmol). The mixture was degassed and purged with N₂ three times and stirred at 100° C. for 16 hr under N₂ atmosphere. The reaction mixture was diluted with H₂O (100 mL) and extracted with CH₂Cl₂ (100 mL×2). The combined organic layers were washed with brine (100 mL), dried over Na₂SO₄, filtered and concentrated under reduced pressure. After purified by silica gel flash chromatography (Eluent of 20%˜70% EtOAc in petroleum ether), 3-(3-(4-(((tert-butyldimethylsilyl)oxy)methyl)phenyl)-5-(3-methyl-1H-pyrazol-5-yl)-3H-imidazo[4,5-b]pyridin-2-yl)pyridin-2-amine (240 mg, yield: 41%) was obtained as a yellow solid. MS: m/z=512.2 [M+H]⁺. ¹H NMR (400 MHz, Dimethylsulfoxide-d₆) δ 12.64 (s, 1H), 8.17 (d, J=8.4 Hz, 1H), 8.00-7.93 (m, 2H), 7.49-7.43 (m, 4H), 7.18-7.13 (m, 1H), 7.00 (s, 2H), 6.40-6.34 (m, 2H), 4.82 (s, 2H), 2.24 (s, 3H), 0.93 (s, 9H), 0.11 (s, 6H).

### Step 2: (4-(2-(2-Aminopyridin-3-yl)-5-(3-methyl-1H-pyrazol-5-yl)-3H-imidazo[4,5-b]pyridin-3-yl)phenyl)methanol

To a solution of 3-(3-(4-(((tert-butyldimethylsilyl)oxy)methyl)phenyl)-5-(3-methyl-1H-pyrazol-5-yl)-3H-imidazo[4,5-b]pyridin-2-yl)pyridin-2-amine (240 mg, 469 μmol) in THF (5 mL) was added TBAF (1 M, 0.9 mL). The mixture was stirred at 25° C. for 1 hr. The reaction mixture was diluted with H₂O (20 mL) and extracted with CH₂Cl₂ (20 mL×2). The combined organic layers were washed with brine (20 mL×2), dried over Na₂SO₄, filtered and concentrated under reduced pressure, (4-(2-(2-aminopyridin-3-yl)-5-(3-methyl-1H-pyrazol-5-yl)-3H-imidazo[4,5-b]pyridin-3-yl)phenyl)methanol (110 mg, yield: 59%) was obtained as a yellow solid. MS: m/z=398.0 [M+H]⁺. ¹H NMR (400 MHz, Dimethylsulfoxide-d₆) δ 12.64 (s, 1H), 8.22-8.12 (m, 1H), 8.01-7.89 (m, 2H), 7.50-7.40 (m, 4H), 7.19 (dd, J=7.6, 1.6 Hz, 1H), 6.95 (s, 2H), 6.45-6.29 (m, 2H), 5.35 (t, J=5.6 Hz, 1H), 4.60 (d, J=5.6 Hz, 2H), 2.24 (s, 3H).

### Step 3: 3-(3-(4-(Chloromethyl)phenyl)-5-(3-methyl-1H-pyrazol-5-yl)-3H-imidazo[4,5-b]pyridin-2-yl)pyridin-2-amine

To a solution of (4-(2-(2-aminopyridin-3-yl)-5-(3-methyl-1H-pyrazol-5-yl)-3H-imidazo[4,5-b]pyridin-3-yl)phenyl)methanol (110 mg, 277 μmol) in CH₂Cl₂ (2 mL) was added SOCl₂ (98.8 mg, 830 μmol). The mixture was stirred at 40° C. for 0.5 hr. The reaction was concentrated under reduced pressure, 3-(3-(4-(chloromethyl)phenyl)-5-(3-methyl-1H-pyrazol-5-yl)-3H-imidazo[4,5-b]pyridin-2-yl)pyridin-2-amine (120 mg, crude) was obtained as a yellow solid. MS: m/z=416.0 [M+H]⁺.

### Step 4: 4-((1-(4-(2-(2-Aminopyridin-3-yl)-5-(3-methyl-1H-pyrazol-5-yl)-3H-imidazo[4,5-b]pyridin-3-yl)benzyl)piperidin-4-yl)amino)pyrimidine-2-carbonitrile

To a solution of 3-(3-(4-(chloromethyl)phenyl)-5-(3-methyl-1H-pyrazol-5-yl)-3H-imidazo[4,5-b]pyridin-2-yl)pyridin-2-amine (120 mg, 289 μmol) and Intermediate 51 (91.6 mg, 289 μmol, TFA salt) in DMF (2 mL) were added K₂CO₃ (199 mg, 1.44 mmol) and NaI (4.33 mg, 28.9 μmol). The mixture was stirred at 60° C. for 1 hr. After purified by prep-HPLC (column: Waters xbridge 150×25 mm×10 μm; mobile phase: [water (NH₄HCO₃)-ACN]; gradient: 27%-57% B over 10 min), 4-((1-(4-(2-(2-aminopyridin-3-yl)-5-(3-methyl-1H-pyrazol-5-yl)-3H-imidazo[4,5-b]pyridin-3-yl)benzyl)piperidin-4-yl)amino)pyrimidine-2-carbonitrile (Example 465, 28.6 mg, yield: 16% for two steps) was obtained as a light yellow solid. MS: m/z=583.3 [M+H]⁺. ¹H NMR (400 MHz, Dimethylsulfoxide-d₆) δ 12.64 (s, 1H), 8.21-7.92 (m, 5H), 7.52-7.39 (m, 4H), 7.14-7.09 (m, 1H), 6.99 (s, 2H), 6.67 (d, J=5.6 Hz, 1H), 6.48-6.23 (m, 2H), 3.88-3.75 (m, 1H), 3.58 (s, 2H), 2.89-2.80 (m, 2H), 2.24 (s, 3H), 2.17-2.09 (m, 2H), 1.94-1.84 (m, 2H), 1.54-1.45 (m, 2H).

### Example 466: 4-((1-(4-(2-(2-Aminopyridin-3-yl)-5-(1-ethyl-1H-pyrazol-4-yl)-3H-imidazo[4,5-b]pyridin-3-yl)benzyl)piperidin-4-yl)amino)pyrinidine-2-carbonitrile

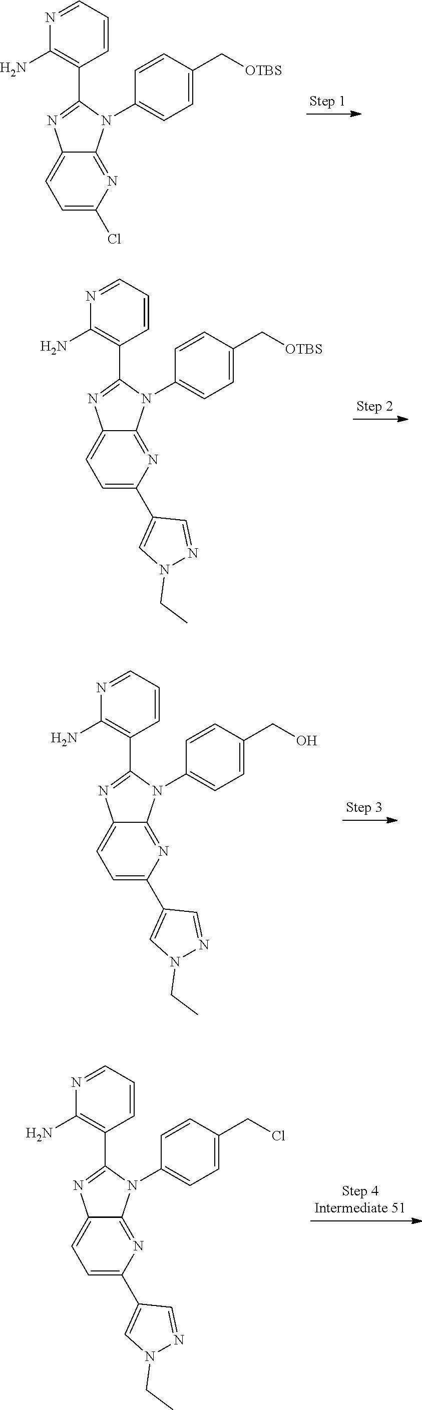

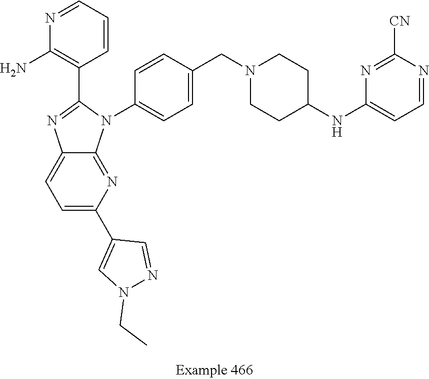 [SMILES: CCn1cc(-c2ccc3nc(-c4cccnc4N)n(-c4ccc(CN5CCC(Nc6ccnc(C#N)n6)CC5)cc4)c3n2)cn1]

### Step 1: 3-(3-(4-(((Tert-butyldimethylsilyl)oxy)methyl)phenyl)-5-(1-ethyl-1H-pyrazol-4-yl)-3H-imidazo[4,5-b]pyridin-2-yl)pyridin-2-amine

A mixture of 3-(3-(4-(((tert-butyldimethylsilyl)oxy)methyl)phenyl)-5-chloro-3H-imidazo[4,5-b]pyridin-2-yl)pyridin-2-amine (refer to Intermediate 117 for detail procedures, 500 mg, 1.07 mmol), (1-ethyl-1H-pyrazol-4-yl)boronic acid (150 mg, 1.07 mmol), Cs₂CO₃ (1.05 g, 3.22 mmol) and Pd(dppf)Cl₂ (78.5 mg, 107 μmol) in 1,4-dioxane (5 mL) and H₂O (1 mL) was degassed and purged with N₂ three times, and then was stirred at 80° C. for 16 hr under N₂ atmosphere. The reaction mixture was diluted with CH₂Cl₂ (10 mL) and extracted with CH₂Cl₂ (15 mL×3). The combined organic layers were washed with brine (50 mL), dried over Na₂SO₄, filtered, and concentrated under reduced pressure. After purified by silica gel flash chromatography (Eluent of 1%˜50% EtOAc in petroleum ether), 3-(3-(4-(((tert-butyldimethylsilyl)oxy)methyl)phenyl)-5-(1-ethyl-1H-pyrazol-4-yl)-3H-imidazo[4,5-b]pyridin-2-yl)pyridin-2-amine (400 mg, yield: 71%) was obtained as a purple oil. MS: m/z=526.6 [M+H]⁺. ¹H NMR (400 MHz, Dimethylsulfoxide-d₆) δ 8.19-8.13 (m, 2H), 7.98 (dd, J=4.8, 1.6 Hz, 1H), 7.92 (s, 1H), 7.68 (d, J=8.0 Hz, 1H), 7.51-7.46 (m, 2H), 7.45-7.39 (m, 2H), 7.13 (dd, J=7.6, 1.6 Hz, 1H), 6.99 (br s, 2H), 6.36 (dd, J=7.6, 4.8 Hz, 1H), 4.82 (s, 2H), 4.15 (q, J=7.2 Hz, 2H), 0.93 (s, 9H), 0.11 (s, 6H).

### Step 2: (4-(2-(2-Aminopyridin-3-yl)-5-(1-ethyl-1H-pyrazol-4-yl)-3H-imidazo[4,5-b]pyridin-3-yl)phenyl)methanol

To a solution of 3-(3-(4-(((tert-butyldimethylsilyl)oxy)methyl)phenyl)-5-(1-ethyl-1H-pyrazol-4-yl)-3H-imidazo[4,5-b]pyridin-2-yl)pyridin-2-amine (400 mg, 761 μmol) in THF (6 mL) was added TBAF (1.52 mmol, 1 M in THF). The mixture was stirred at 25° C. for 1 hr. The reaction mixture was quenched with H₂O (10 mL) at 25° C., and extracted with CH₂Cl₂ (20 mL×3). The combined organic layers were washed with brine (50 mL), dried over Na₂SO₄, filtered and concentrated under reduced pressure. After purified by silica gel flash chromatography (Eluent of 1%˜80% EtOAc in petroleum ether), (4-(2-(2-aminopyridin-3-yl)-5-(1-ethyl-1H-pyrazol-4-yl)-3H-imidazo[4,5-b]pyridin-3-yl)phenyl)methanol (120 mg, yield: 38%) was obtained as a yellow solid. MS: m/z=412.4 [M+H]⁺. ¹H NMR (400 MHz, Dimethylsulfoxide-d₆) δ 8.20-8.12 (m, 2H), 8.07-7.94 (m, 2H), 7.70 (d, J=8.0 Hz, 1H), 7.52-7.45 (m, 2H), 7.45-7.38 (m, 2H), 7.28-6.84 (m, 3H), 6.51-6.38 (m, 1H), 5.57-5.17 (m, 1H), 4.61 (s, 2H), 4.16 (q, J=7.2 Hz, 2H), 1.38 (t, J=7.6 Hz, 3H).

### Step 3: 3-(3-(4-(Chloromethyl)phenyl)-5-(1-ethyl-1H-pyrazol-4-yl)-3H-imidazo[4,5-b]pyridin-2-yl)pyridin-2-amine

To a solution of (4-(2-(2-aminopyridin-3-yl)-5-(1-ethyl-1H-pyrazol-4-yl)-3H-imidazo[4,5-b]pyridin-3-yl)phenyl)methanol (120 mg, 292 μmol) in CH₂Cl₂ (5 mL) was added SOCl₂ (69.4 mg, 583 μmol) at 0° C. The mixture was stirred at 40° C. for 0.5 hr. The reaction mixture was quenched with H₂O (1 mL) at 25° C., and concentrated under reduced pressure, 3-(3-(4-(chloromethyl)phenyl)-5-(1-ethyl-1H-pyrazol-4-yl)-3H-imidazo[4,5-b]pyridin-2-yl)pyridin-2-amine (136 mg, HCl salt) was obtained as a yellow solid. MS: m/z=430.1 [M+H]⁺.

### Step 4: 4-((1-(4-(2-(2-Aminopyridin-3-yl)-5-(1-ethyl-1H-pyrazol-4-yl)-3H-imidazo[4,5-b]pyridin-3-yl)benzyl)piperidin-4-yl)amino)pyrimidine-2-carbonitrile

To a solution of 3-(3-(4-(chloromethyl)phenyl)-5-(1-ethyl-1H-pyrazol-4-yl)-3H-imidazo[4,5-b]pyridin-2-yl)pyridin-2-amine (136 mg, 292 μmol, HCl salt) and Intermediate 51 (92.5 mg, 292 μmol, TFA) in DMF (2 mL) were added K₂CO₃ (202 mg, 1.46 mmol) and NaI (8.74 mg, 58.3 μmol). The mixture was stirred at 50° C. for 16 hr. The mixture was filtered through Celite, and the filtrate was concentrated. After purified by prep-HPLC(column: Waters xbridge 150×25 mm×10 μm; mobile phase: [water (NH₃H₂O+NH₄HCO₃)-ACN]; gradient: 32%-62% B over 11 min), 4-((1-(4-(2-(2-aminopyridin-3-yl)-5-(1-ethyl-1H-pyrazol-4-yl)-3H-imidazo[4,5-b]pyridin-3-yl)benzyl)piperidin-4-yl)amino)pyrimidine-2-carbonitrile (Example 466, 39 mg, yield: 24% for two steps) was obtained as a light yellow lyophilized powder. MS: m/z=597.4 [M+H]⁺. ¹H NMR (400 MHz, Dimethylsulfoxide-d₆) δ 8.20-8.13 (m, 2H), 8.02-8.11 (m, 2H), 7.98 (dd, J=4.8, 2.0 Hz, 1H), 7.93 (s, 1H), 7.68 (d, J=8.0 Hz, 1H), 7.50-7.44 (m, 2H), 7.43-7.38 (m, 2H), 7.10 (dd, J=7.6, 1.6 Hz, 1H), 7.01 (br s, 2H), 6.68 (d, J=6.0 Hz, 1H), 6.35 (dd, 1=7.6, 4.8 Hz, 1H), 4.15 (q, J=7.2 Hz, 2H), 3.91-3.72 (m, 1H), 3.59 (s, 2H), 2.90-2.79 (m, 2H), 2.22-2.11 (m, 2H), 1.94-1.83 (m, 2H), 1.55-1.45 (m, 2H), 1.37 (t, J=7.6 Hz, 3H).

### Example 467: 4-((1-(4-(2-(2-Aminopyridin-3-yl)-5-(1-methyl-1H-pyrazol-5-yl)-3H-imidazo[4,5-b]pyridin-3-yl)benzyl)piperidin-4-yl)amino)pyrimidine-2-carbonitrile

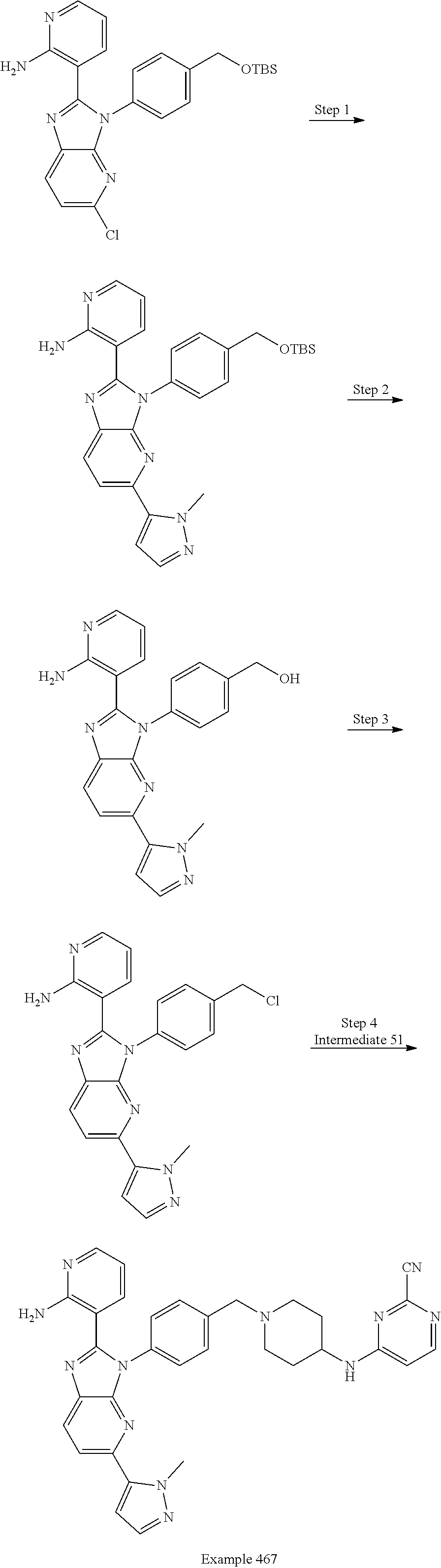 [SMILES: CC(C)(C)[Si](C)(C)OCc1ccc(-n2c(-c3cccnc3N)nc3ccc(Cl)nc32)cc1 ; Cn1nccc1-c1ccc2nc(-c3cccnc3N)n(-c3ccc(CO[Si](C)(C)C(C)(C)C)cc3)c2n1 ; Cn1nccc1-c1ccc2nc(-c3cccnc3N)n(-c3ccc(CO)cc3)c2n1 ; Cn1nccc1-c1ccc2nc(-c3cccnc3N)n(-c3ccc(CCl)cc3)c2n1 ; Cn1nccc1-c1ccc2nc(-c3cccnc3N)n(-c3ccc(CN4CCC(Nc5ccnc(C#N)n5)CC4)cc3)c2n1]

### Step 1: 3-(3-(4-(((Tert-butyldimethylsilyl)oxy)methyl)phenyl)-5-(1-methyl-1H-pyrazol-5-yl)-3H-imidazo[4,5-b]pyridin-2-yl)pyridin-2-amine

To a solution of 3-(3-(4-(((tert-butyldimethylsilyl)oxy)methyl)phenyl)-5-chloro-3H-imidazo[4,5-b]pyridin-2-yl)pyridin-2-amine (refer to Intermediate 117 for detail procedures, 500 mg, 1 mmol) in 1,4-dioxane (5 mL) were added H₂O (1 mL), K₂CO₃ (445 mg, 3.2 mmol), (1-methyl-1H-pyrazol-5-yl)boronic acid (135 mg, 1 mmol) and Pd(dppf)Cl₂ (78 mg, 107 μmol) at 25° C. The mixture was degassed and purged with N₂ three times. The mixture was stirred at 85° C. for 12 hr under N₂. The reaction mixture was poured into H₂O (50 mL) and extracted with CH₂Cl₂ (20 mL×2). The combined organic layers were washed with brine (40 mL), dried over anhydrous Na₂SO₄, filtered and concentrated. After purified by silica gel flash chromatography (Eluent of 0˜40% EtOAc in petroleum ether), 3-(3-(4-(((tert-butyldimethylsilyl)oxy)methyl)phenyl)-5-(1-methyl-1H-pyrazol-5-yl)-3H-imidazo[4,5-b]pyridin-2-yl)pyridin-2-amine (300 mg, yield: 46%) was obtained as a yellow solid. MS: m/z=512.3 [M+H]⁺. ¹H NMR (400 MHz, Dimethylsulfoxide-d₆) δ 8.28 (d, J=8.4 Hz, 1H), 8.01 (dd, J=4.8, 1.6 Hz, 1H), 7.81 (d, J=8.4 Hz, 1H), 7.48-7.46 (m, 5H), 7.23 (dd, J=7.6, 1.6 Hz, 1H), 7.04 (s, 2H), 6.83 (d, J=2.0 Hz, 1H), 6.40 (dd, J=8.0, 4.8 Hz, 1H), 4.80 (s, 2H), 3.98 (s, 3H), 0.91 (s, 9H), 0.09 (s, 6H).

### Step 2: (4-(2-(2-Aminopyridin-3-yl)-5-(I-methyl-1H-pyrazol-5-yl)-3H-imidazo[4,5-b]pyridin-3-yl)phenyl)methanol

To a solution of 3-(3-(4-(((tert-butyldimethylsilyl)oxy)methyl)phenyl)-5-(1-methyl-1H-pyrazol-5-yl)-3H-imidazo[4,5-b]pyridin-2-yl)pyridin-2-amine (300 mg, 586 μmol) in THF (5 mL) was added TBAF (1.8 mL, 1 M) at 25° C. The mixture was stirred at 25° C. for 1 hr. The reaction mixture was poured into H₂O (20 mL). The mixture was extracted with CH₂Cl₂ (20 mL×2). The combined organic layers were washed with brine (40 mL), dried over anhydrous Na₂SO₄, filtered and concentrated, (4-(2-(2-aminopyridin-3-yl)-5-(1-methyl-1H-pyrazol-5-yl)-3H-imidazo[4,5-b]pyridin-3-yl)phenyl)methanol (220 mg, crude) was obtained as a yellow solid. MS: m/z=398.4 [M+H]⁺. ¹H NMR (400 MHz, Dimethylsulfoxide-d₆) δ 8.28 (d, J=8.0 Hz, 1H), 8.01 (dd, J=4.8, 2.0 Hz, 1H), 7.80 (d, J=8.4 Hz, 1H), 7.49-7.43 (m, 5H), 7.24 (dd, J=8.0, 2.0 Hz, 1H), 6.99 (s, 2H), 6.82 (d, J=2.0 Hz, 1H), 6.43 (dd, J=8.0, 5.2 Hz, 1H), 5.42-5.29 (m, 1H), 4.59 (s, 2H), 4.01 (s, 3H).

### Step 3: 3-(3-(4-(Chloromethyl)phenyl)-5-(1-methyl-1H-pyrazol-5-yl)-3H-imidazo[4,5-b]pyridin-2-yl)pyridin-2-amine

To a solution of (4-(2-(2-aminopyridin-3-yl)-5-(1-methyl-1H-pyrazol-5-yl)-3H-imidazo[4,5-b]pyridin-3-yl)phenyl)methanol (200 mg, 503 μmol) in CH₂Cl₂ (5 mL) was added SOCl₂ (299 mg, 2.5 mmol) at 25° C., the mixture was stirred at 40° C. for 0.5 hr. The reaction mixture was concentrated under reduced pressure, 3-(3-(4-(chloromethyl)phenyl)-5-(1-methyl-1H-pyrazol-5-yl)-3H-imidazo[4,5-b]pyridin-2-yl)pyridin-2-amine (200 mg, yield: 90% for 2 steps) was obtained as a yellow solid, which was used in the next step without further purification. MS: m/z=415.9 [M+H]⁺. ¹H NMR (400 MHz, Dimethylsulfoxide-d₆) δ 8.56-8.46 (m, 1H), 8.38 (d, J=8.4 Hz, 1H), 8.16 (dd, J=6.4, 1.2 Hz, 1H), 7.94-7.87 (m, 2H), 7.66-7.58 (m, 5H), 7.49 (d, J=2.0 Hz, 1H), 6.92 (dd, J=7.2, 6.4 Hz, 1H), 6.88 (d, J=2.0 Hz, 1H), 4.86 (s, 2H), 4.02 (s, 3H).

### Step 4: 4-((1-(4-(2-(2-Aminopyridin-3-yl)-5-(1-methyl-1H-pyrazol-5-yl)-3H-imidazo[4,5-b]pyridin-3-yl)benzyl)piperidin-4-yl)amino)pyrimidine-2-carbonitrile

To a solution of 3-(3-(4-(chloromethyl)phenyl)-5-(1-methyl-1H-pyrazol-5-yl)-3H-imidazo[4,5-b]pyridin-2-yl)pyridin-2-amine (100 mg, 240 μmol) in DMF (3 mL) were added Intermediate 51 (59 mg, 289 μmol), NaI (36 mg, 240 μmol) and K₂CO₃ (133 mg, 962 μmol) at 25° C., the mixture was stirred at 50° C. for 1 hr. The reaction mixture was poured into H₂O (50 mL) and extracted with CH₂Cl₂ (20 mL×2). The combined organic layers were washed with brine (40 mL), dried over anhydrous Na₂SO₄, filtered and concentrated. After purified by silica gel flash chromatography (Eluent of 0˜6% MeOH in CH₂Cl₂), 4-((1-(4-(2-(2-aminopyridin-3-yl)-5-(1-methyl-1H-pyrazol-5-yl)-3H-imidazo[4,5-b]pyridin-3-yl)benzyl)piperidin-4-yl)amino)pyrimidine-2-carbonitrile (Example 467, 46.5 mg, yield: 32%) was obtained as a yellow solid. MS: m/z=583.4 [M+H]⁺. ¹H NMR (400 MHz, Dimethylsulfoxide-d₆) δ 8.29 (d, J=8.4 Hz, 1H), 8.13-7.98 (m, 3H), 7.81 (d, J=8.4 Hz, 1H), 7.51-7.40 (m, 5H), 7.23 (dd, J=7.2, 1.6 Hz, 1H), 7.08 (s, 2H), 6.84 (d, J=2.0 Hz, 1H), 6.67 (d, J=6.0 Hz, 1H), 6.41 (dd, J=7.6, 4.8 Hz, 1H), 3.98 (s, 3H), 3.88-3.74 (m, 1H), 3.63-3.57 (m, 2H), 2.86-2.74 (m, 2H), 2.21-2.07 (m, 2H), 1.92-1.85 (m, 2H), 1.55-1.44 (m, 2H).

### Example 468: 4-((1-(4-(2-(2-Aminopyridin-3-yl)-5-(3-(2-oxopyrrolidin-1-yl)phenyl)-3H-imidazo[4,5-b]pyridin-3-yl)benzyl)piperidin-4-yl)amino)pyrimidine-2-carbonitrile

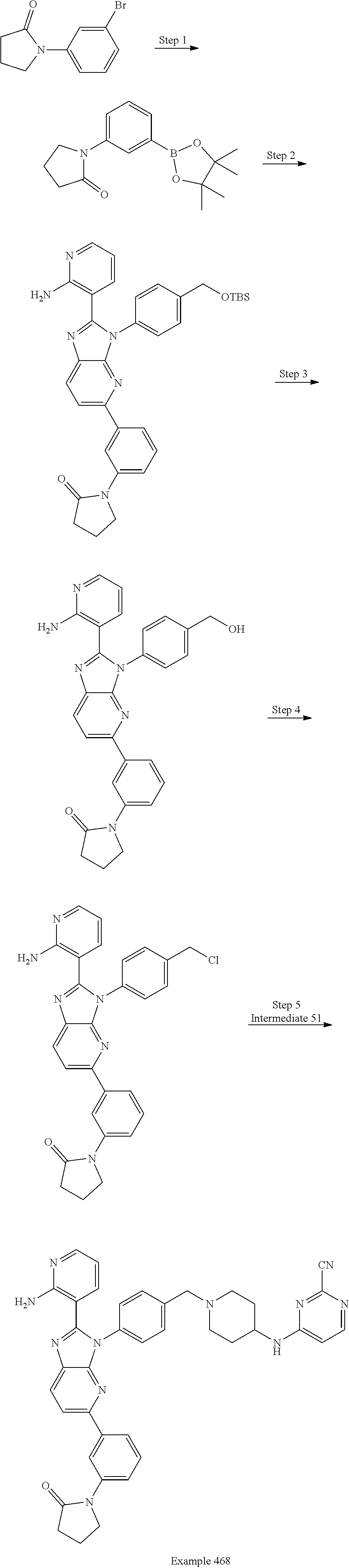 [SMILES: O=C1CCCN1c1cccc(Br)c1 ; CC1(C)OB(c2cccc(N3CCCC3=O)c2)OC1(C)C ; CC(C)(C)[Si](C)(C)OCc1ccc(-n2c(-c3cccnc3N)nc3ccc(-c4cccc(N5CCCC5=O)c4)nc32)cc1 ; Nc1ncccc1-c1nc2ccc(-c3cccc(N4CCCC4=O)c3)nc2n1-c1ccc(CO)cc1 ; Nc1ncccc1-c1nc2ccc(-c3cccc(N4CCCC4=O)c3)nc2n1-c1ccc(CCl)cc1 ; N#Cc1nccc(NC2CCN(Cc3ccc(-n4c(-c5cccnc5N)nc5ccc(-c6cccc(N7CCCC7=O)c6)nc54)cc3)CC2)n1]

### Step 1: 1-(3-(4,4,5,5-Tetramethyl-1,3,2-dioxaborolan-2-yl)phenyl)pyrrolidin-2-one

A mixture of 1-(3-bromophenyl)pyrrolidin-2-one (300 mg, 1.25 mmol), 4,4,4′,4′,5,5,5′,5′-octamethyl-2,2′-bi(1,3,2-dioxaborolane) (349 mg, 1.37 mmol), Pd(dppf)Cl₂ (91.4 mg, 125 μmol) and AcOK (368 mg, 3.75 mmol) in 1,4-dioxane (5 mL) and H₂O (1 mL) was degassed and purged with N₂ three times, and was stirred at 80° C. for 16 hr under N₂ atmosphere. The mixture was used in the next step without work-up and purification. 1-(3-(4,4,5,5-Tetramethyl-1,3,2-dioxaborolan-2-yl)phenyl)pyrrolidin-2-one (358 mg, crude) was obtained as a black oil. MS: m/z=288.0 [M+H]⁺.

### Step 2: 1-(3-(2-(2-Aminopyridin-3-yl)-3-(4-(((tert-butyldimethylsilyl)oxy)methyl)phenyl)-3H-imidazo[4,5-b]pyridin-5-yl)phenyl)pyrrolidin-2-one

A mixture of 1-(3-(4,4,5,5-tetramethyl-1,3,2-dioxaborolan-2-yl)phenyl)pyrrolidin-2-one (358 mg, 1.25 mmol), 3-(3-(4-(((tert-butyldimethylsilyl)oxy)methyl)phenyl)-5-chloro-3H-imidazo[4,5-b]pyridin-2-yl)pyridin-2-amine (refer to Intermediate 17 for detail procedures, 581 mg, 1.25 mmol), Pd(dppf)Cl₂ (91.2 mg, 125 μmol) and Cs₂CO₃ (1.22 g, 3.74 mmol) in 1,4-dioxane (5 mL) and H₂O (1 mL) was degassed and purged with N₂ three times, and then was stirred at 80° C. for 16 hr under N₂ atmosphere. The reaction mixture was quenched with H₂O (10 mL) and extracted with CH₂Cl₂ (15 mL×3). The combined organic layers were washed with brine (50 mL), dried over Na₂SO₄, filtered, and concentrated under reduced pressure. After purified by silica gel flash chromatography (Eluent of 1%˜80% EtOAc in petroleum ether), 1-(3-(2-(2-aminopyridin-3-yl)-3-(4-(((tert-butyldimethylsilyl)oxy)methyl)phenyl)-3H-imidazo[4,5-b]pyridin-5-yl)phenyl)pyrrolidin-2-one (500 mg, yield: 60%) was obtained as a brown solid. MS: m/z=591.2 [M+H]⁺. ¹H NMR (400 MHz, Dimethylsulfoxide-d₆) δ 8.20-8.15 (m, 2H), 7.90 (dd, J=4.8, 1.6 Hz, 1H), 7.85 (d, J=8.4 Hz, 1H), 7.67 (d, J=8.0 Hz, 1H), 7.60-7.57 (m, 1H), 7.38-7.30 (m, 5H), 7.09 (dd, J=7.6, 1.6 Hz, 1H), 6.90 (br s, 2H), 6.28 (dd, J=7.6, 4.8 Hz, 1H), 4.71 (s, 2H), 3.80-3.76 (m, 2H), 2.03-1.92 (m, 4H), 0.84-0.80 (m, 9H), 0.00 (s, 6H).

### Step 3: 1-(3-(2-(2-Aminopyridin-3-yl)-3-(4-(hydroxymethyl)phenyl)-3H-imidazo[4,5-b]pyridin-5-yl)phenyl)pyrrolidin-2-one

To a solution of 1-(3-(2-(2-aminopyridin-3-yl)-3-(4-(((tert-butyldimethylsilyl)oxy)methyl)phenyl)-3H-imidazo[4,5-b]pyridin-5-yl)phenyl)pyrrolidin-2-one (600 mg, 1.02 mmol) in THF (5 mL) was added TBAF (2.03 mmol, 1 M). The mixture was stirred at 25° C. for 1 hr. The reaction mixture was quenched with H₂O (10 mL) at 25° C., and extracted with CH₂Cl₂ (20 mL×3). The combined organic layers were washed with brine (50 mL), dried over Na₂SO₄, filtered and concentrated under reduced pressure. After purified by silica gel flash chromatography (Eluent of 1%˜100% EtOAc in petroleum ether), 1-(3-(2-(2-aminopyridin-3-yl)-3-(4-(hydroxymethyl)phenyl)-3H-imidazo[4,5-b]pyridin-5-yl)phenyl)pyrrolidin-2-one (200 mg, yield: 40%) was obtained as a yellow solid. MS: m/z=477.1 [M+H]⁺.

### Step 4: 1-(3-(2-(2-Aminopyridin-3-yl)-3-(4-(chloromethyl)phenyl)-3H-imidazo[4,5-b]pyridin-5-yl)phenyl)pyrrolidin-2-one

To a solution of 1-(3-(2-(2-Aminopyridin-3-yl)-3-(4-(hydroxymethyl)phenyl)-3H-imidazo[4,5-b]pyridin-5-yl)phenyl)pyrrolidin-2-one (230 mg, 483 μmol) in CH₂Cl₂ (5 mL) was added SOCl₂ (115 mg, 965 μmol) at 0° C. The mixture was stirred at 40° C. for 0.5 hr. The reaction mixture was quenched with H₂O (1 mL) at 25° C. and concentrated under reduced pressure to give a residue. And the reaction mixture was diluted with aqueous Na₂CO₃ (10 mL) and extracted with CH₂Cl₂ (15 mL×2). The combined organic layers were washed with brine (15 mL), dried over Na₂SO₄, filtered and concentrated under reduced pressure, 1-(3-(2-(2-aminopyridin-3-yl)-3-(4-(chloromethyl)phenyl)-3H-imidazo[4,5-b]pyridin-5-yl)phenyl)pyrrolidin-2-one (180 mg) was obtained as a yellow solid. MS: m/z=495.0 [M+H]⁺. ¹H NMR (400 MHz, Dimethylsulfoxide-d₆) δ 8.32-8.26 (m, 2H), 8.02 (dd, J=4.8, 1.6 Hz, 1H), 7.97 (d, J=8.4 Hz, 1H), 7.79 (d, J=8.0 Hz, 1H), 7.71 (dd, J=8.0, 1.2 Hz, 1H), 7.64-7.59 (m, 2H), 7.56-7.52 (m, 2H), 7.49-7.43 (m, 1H), 7.32 (d, J=7.6 Hz, 1H), 7.08 (br s, 2H), 6.52-6.47 (m, 1H), 4.87 (s, 2H), 3.89 (t, 1=7.2 Hz, 2H), 2.55-2.52 (m, 2H), 2.11-2.07 (m, 2H).

### Step 5: 4-((1-(4-(2-(2-Aminopyridin-3-yl)-5-(3-(2-oxopyrrolidin-1-yl)phenyl)-3H-imidazo[4,5-b]pyridin-3-yl)benzyl)piperidin-4-yl)amino)pyrimidine-2-carbonitrile

To a solution of 1-(3-(2-(2-aminopyridin-3-yl)-3-(4-(chloromethyl)phenyl)-3H-imidazo[4,5-b]pyridin-5-yl)phenyl)pyrrolidin-2-one (180 mg, 364 μmol) and Intermediate 51 (115 mg, 364 μmol) in DMF (5 mL) were added K₂CO₃ (151 mg, 1.09 mmol) and NaI (10.9 mg, 72.7 μmol). The mixture was stirred at 50° C. for 16 hr. The mixture was filtered through Celite, and the filtrate was concentrated. After purified by prep-HPLC(column: Waters xbridge 150×25 mm×10 μm; mobile phase: [water (NH₃H₂O+NH₄HCO₃)-ACN]; gradient: 37%-67% B over 11 min), 4-((1-(4-(2-(2-aminopyridin-3-yl)-5-(3-(2-oxopyrrolidin-1-yl)phenyl)-3H-imidazo[4,5-b]pyridin-3-yl)benzyl)piperidin-4-yl)amino)pyrimidine-2-carbonitrile (Example 468, 41.8 mg, yield: 17% for two steps) was obtained as a light-yellow lyophilized powder. MS: m/z=662.4 [M+H]⁺. ¹H NMR (400 MHz, Dimethylsulfoxide-d₆) δ 8.30-8.26 (m, 2H), 8.09-8.05 (m, 2H), 8.00 (dd, J=4.8, 1.6 Hz, 1H), 7.97 (d, J=8.4 Hz, 1H), 7.79 (d, J=8.0 Hz, 1H), 7.69 (dd, J=8.0, 1.2 Hz, 1H), 7.49-7.42 (m, 5H), 7.18 (dd, J=7.6, 2.0 Hz, 1H), 7.04 (br s, 2H), 6.68 (br d, J=6.0 Hz, 1H), 6.39 (dd, J=8.0, 5.2 Hz, 1H), 3.87-3.75 (m, 3H), 3.59 (s, 2H), 2.86-2.78 (m, 2H), 2.49-2.47 (m, 2H), 2.21-2.12 (m, 2H), 2.10-2.04 (m, 2H), 1.95-1.86 (m, 2H), 1.59-1.48 (m, 2H).

### Example 469: 4-((1-(4-(2-(2-Aminopyridin-3-yl)-5-(4-fluoro-2-methoxyphenyl)-3H-imidazo[4,5-b]pyridin-3-yl)benzyl)piperidin-4-yl)amino)pyrimidine-2-carbonitrile

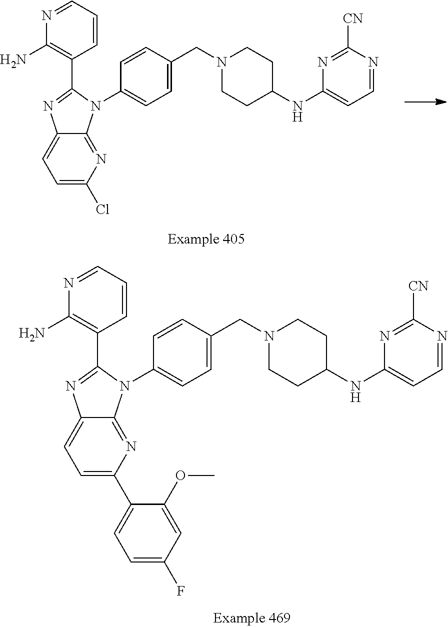 [SMILES: N#Cc1nccc(NC2CCN(Cc3ccc(-n4c(-c5cccnc5N)nc5ccc(Cl)nc54)cc3)CC2)n1 ; COc1cc(F)ccc1-c1ccc2nc(-c3cccnc3N)n(-c3ccc(CN4CCC(Nc5ccnc(C#N)n5)CC4)cc3)c2n1]

A mixture of Example 405 (200 mg, 372 μmol), (4-fluoro-2-methoxyphenyl)boronic acid (63.3 mg, 372 μmol), Cs₂CO₃ (364 mg, 1.12 mmol), Pd(dppf)Cl₂ (54.5 mg, 74.5 μmol) in 1,4-dioxane (2.5 mL) and H₂O (0.5 mL) were degassed and purged with N₂ three times, and then was stirred at 80° C. for 16 hr under N₂ atmosphere. The reaction mixture was filtered and concentrated under reduced pressure. After purified by prep-HPLC (column: Waters xbridge 150×25 mm 10 μm; mobile phase: [water (NH₄HCO₃)-ACN]; gradient: 44%-64% B over 11 min), 4-((1-(4-(2-(2-aminopyridin-3-yl)-5-(4-fluoro-2-methoxyphenyl)-3H-imidazo[4,5-b]pyridin-3-yl)benzyl)piperidin-4-yl)amino)pyrimidine-2-carbonitrile (Example 469, 37.4 mg, yield: 15%) was obtained as a light yellow lyophilized powder. MS: m/z=627.5 [M+H]⁺. ¹H NMR (400 MHz, Methanol-d₄) δ 8.13 (d, J=8.4 Hz, 1H), 8.05-7.95 (m, 2H), 7.88 (d, J=8.4 Hz, 1H), 7.71-7.67 (m, 1H), 7.52 (d, J=8.4 Hz, 2H), 7.43 (d, J=8.4 Hz, 2H), 7.32 (dd, J=8.0, 2.0 Hz, 1H), 6.90 (dd, J=11.2, 2.4 Hz, 1H), 6.73 (td, J=16.0, 2.4 Hz, 1H), 6.60-6.58 (m, 1H), 6.47 (dd, J=7.6, 4.8 Hz, 1H), 3.99-3.91 (m, 1H), 3.88 (s, 3H), 3.65 (s, 2H), 2.97-2.91 (m, 2H), 2.34-2.20 (m, 2H), 2.07-1.93 (m, 2H), 1.68-1.55 (m, 2H). ¹⁹F NMR (400 MHz, Methanol-d₄) δ −112.4.

### Example 470: 4-((1-(4-(2-(2-Aminopyridin-3-yl)-5-(2-(trifluoromethyl)phenyl)-3H-imidazo[4,5-b]pyridin-3-yl)benzyl)piperidin-4-yl)amino)pyrimidine-2-carbonitrile

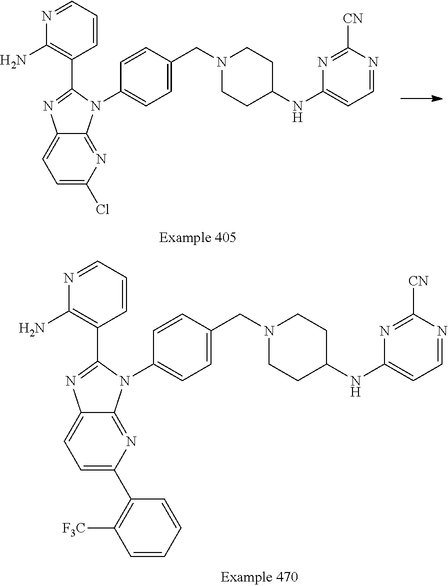 [SMILES: N#Cc1nccc(NC2CCN(Cc3ccc(-n4c(-c5cccnc5N)nc5ccc(Cl)nc54)cc3)CC2)n1 ; N#Cc1nccc(NC2CCN(Cc3ccc(-n4c(-c5cccnc5N)nc5ccc(-c6ccccc6C(F)(F)F)nc54)cc3)CC2)n1]

A mixture of Example 405 (200 mg, 372 μmol), (2-(trifluoromethyl)phenyl)boronic acid (70.7 mg, 372 μmol), Cs₂CO₃ (364 mg, 1.12 mmol), and Pd(dppf)Cl₂ (54.5 mg, 74.5 μmol) in 1,4-dioxane (2.5 mL) and H₂O (0.5 mL) were degassed and purged with N₂ three times, and then was stirred at 80° C. for 16 hr under N₂ atmosphere. The reaction mixture was filtered and concentrated under reduced pressure. After purified by prep-HPLC (column: Waters xbridge 150×25 mm 10 μm; mobile phase: [water (NH₃H₂O)-ACN]; gradient: 47%-77% B over 15 min), 4-((1-(4-(2-(2-Aminopyridin-3-yl)-5-(2-(trifluoromethyl)phenyl)-3H-imidazo[4,5-b]pyridin-3-yl)benzyl)piperidin-4-yl)amino)pyrimidine-2-carbonitrile (Example 470, 64.1 mg, yield: 26%) was obtained as a gray lyophilized powder. MS: m/z=647.3 [M+H]r. ¹H NMR (400 MHz, Dimethylsulfoxide-d₆) δ 8.30 (d, J=8.4 Hz, 1H), 8.11-7.97 (m, 3H), 7.84 (d, J=7.6 Hz, 1H), 7.77-7.72 (m, 1H), 7.67-7.62 (m, 1H), 7.57 (d, J=8.0 Hz, 1H), 7.51 (d, J=8.4 Hz, 1H), 7.39 (q, 1=8.4 Hz, 4H), 7.17 (dd, J=7.6, 1.6 Hz, 1H), 7.02 (br s, 2H), 6.66 (br d, J=6.0 Hz, 1H), 6.38 (dd, J=7.6, 4.4 Hz, 1H), 3.86-3.72 (m, 1H), 3.53 (s, 2H), 2.78 (br d, 1=10.4 Hz, 2H), 2.16-2.07 (m, 2H), 1.90-1.80 (m, 2H), 1.48-1.44 (m, 2H). ¹⁹F NMR (400 MHz, Dimethylsulfoxide-d₆) δ −55.4.

### Example 471: 4-((1-(4-(2-(2-Aminopyridin-3-yl)-5-(4-(trifluoromethyl)phenyl)-3H-imidazo[4,5-b]pyridin-3-yl)benzyl)piperidin-4-yl)amino)pyrimidine-2-carbonitrile

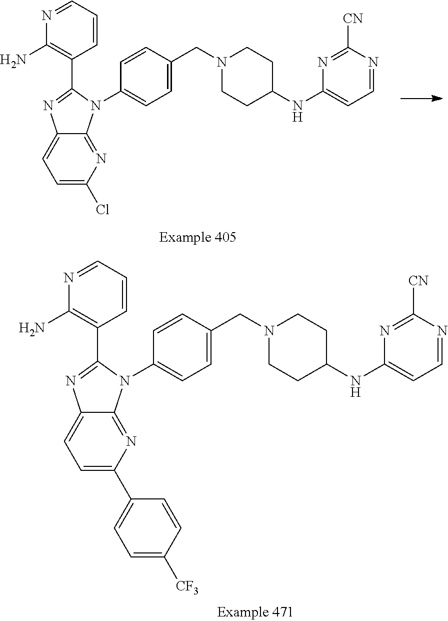 [SMILES: N#Cc1nccc(NC2CCN(Cc3ccc(-n4c(-c5cccnc5N)nc5ccc(Cl)nc54)cc3)CC2)n1 ; N#Cc1nccc(NC2CCN(Cc3ccc(-n4c(-c5cccnc5N)nc5ccc(-c6ccc(C(F)(F)F)cc6)nc54)cc3)CC2)n1]

A mixture of Example 405 (200 mg, 372 μmol), (4-(trifluoromethyl)phenyl)boronic acid (70.7 mg, 372 μmol), Cs₂CO₃ (364 mg, 1.12 mmol), and Pd(dppf)Cl₂ (54.5 mg, 74.5 μmol) in 1,4-dioxane (2.5 mL) and H₂O (0.5 mL) were degassed and purged with N₂ three times, and then was stirred at 80° C. for 16 hr under N₂ atmosphere. The reaction mixture was filtered and concentrated under reduced pressure. After purified by prep-HPLC (column: Waters Xbridge 150×25 mm×5 μm; mobile phase: [water (NH₃H₂O+NH₄HCO₃)-ACN]; gradient: 57%-87% B over 11 min), 4-((1-(4-(2-(2-aminopyridin-3-yl)-5-(4-(trifluoromethyl)phenyl)-3H-imidazo[4,5-b]pyridin-3-yl)benzyl)piperidin-4-yl)amino)pyrimidine-2-carbonitrile (Example 471, 38.1 mg, yield: 15%) was obtained as a gray lyophilized powder. MS: m/z=647.3 [M+H]⁺. ¹H NMR (400 MHz, Dimethylsulfoxide-d₆) δ 8.36-8.21 (m, 3H), 8.12-7.96 (m, 4H), 7.84-7.80 (m, 2H), 7.47 (br s, 4H), 7.16-7.14 (m, 1H), 7.03 (s, 2H), 6.67 (br s, 1H), 6.38 (br s, 1H), 3.88-3.74 (m, 1H), 3.59 (br s, 2H), 2.82-2.81 (m, 2H), 2.15-2.14 (m, 2H), 1.89-1.87 (m, 2H), 1.51-1.41 (m, 2H). ¹⁹F NMR (400 MHz, Dimethylsulfoxide-d₆) δ −61.0.

### Example 472: 4-((1-(4-(2-(2-Aminopyridin-3-yl)-5-(2-methyl-2H-1,2,3-triazol-4-yl)-3H-imidazo[4,5-b]pyridin-3-yl)benzyl)piperidin-4-yl)amino)pyrimidine-2-carbonitrile

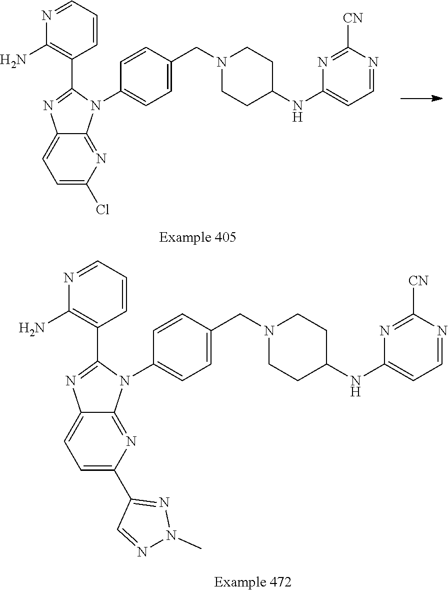 [SMILES: N#Cc1nccc(NC2CCN(Cc3ccc(-n4c(-c5cccnc5N)nc5ccc(Cl)nc54)cc3)CC2)n1 ; Cn1ncc(-c2ccc3nc(-c4cccnc4N)n(-c4ccc(CN5CCC(Nc6ccnc(C#N)n6)CC5)cc4)c3n2)n1]

A mixture of Example 405 (150 mg, 279 μmol), (2-methyl-2H-1,2,3-triazol-4-yl)boronic acid (43 mg, 335 μmol), cataCXiumAPdG3 (118 mg, 540 μmol), K₃PO₄ (119 mg, 559 μmol) and PCy₃ (7.8 mg, 28 μmol) in DMF (3 mL) was degassed and purged with N₂ three times. The mixture was stirred at 90° C. for 16 hr under N₂ atmosphere. The reaction mixture was poured into H₂O (15 mL) and extracted with EtOAc (15 mL×3). The combined organic phases were dried over anhydrous Na₂SO₄, filtered, and concentrated under reduced pressure. After purified by prep-HPLC(column: Waters xbridge 150×25 mm 5 μm; mobile phase: [water (NH₄HCO₃)-ACN]; gradient: 34%-64% B over 11 min), 4-((1-(4-(2-(2-aminopyridin-3-yl)-5-(2-methyl-2H-1,2,3-triazol-4-yl)-3H-imidazo[4,5-b]pyridin-3-yl)benzyl)piperidin-4-yl)amino)pyrimidine-2-carbonitrile (Example 472, 20.5 mg, yield: 12%) was obtained as an off white lyophilized powder. MS: m/z=584.3 [M+H]⁺. ¹H NMR (400 MHz, Dimethylsulfoxide-d₆) δ 8.29 (d, J=8.0 Hz, 1H), 8.13-8.08 (m, 1H), 8.08-8.03 (m, 2H), 8.00 (dd, J=4.8, 1.6 Hz, 1H), 7.95 (d, J=8.0 Hz, 1H), 7.54-7.38 (m, 4H), 7.15 (dd, J=7.6, 2.0 Hz, 1H), 6.99 (br s, 2H), 6.68 (d, J=6.0 Hz, 1H), 6.38 (dd, J=7.6, 4.8 Hz, 1H), 4.22 (s, 3H), 3.90-3.73 (m, 1H), 3.60 (br s, 2H), 2.90-2.80 (m, 2H), 2.20-2.12 (m, 2H), 1.93-1.87 (m, 2H), 1.56-1.46 (m, 2H).

### Example 473: 4-((1-(4-(2-(2-Aminopyridin-3-yl)-5-(3,5-dimethylisoxazol-4-yl)-3H-imidazo[4,5-b]pyridin-3-yl)benzyl)piperidin-4-yl)amino)pyrimidine-2-carbonitrile

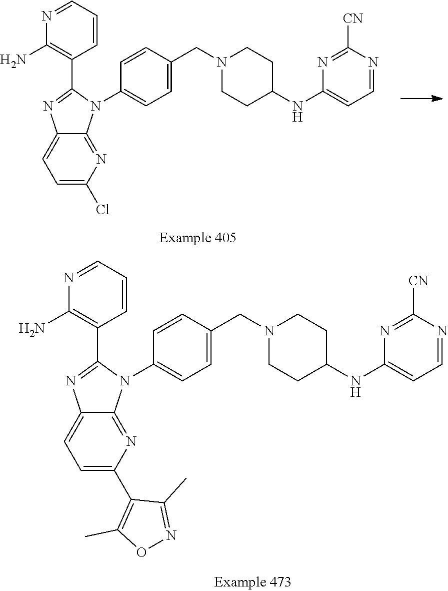 [SMILES: N#Cc1nccc(NC2CCN(Cc3ccc(-n4c(-c5cccnc5N)nc5ccc(Cl)nc54)cc3)CC2)n1 ; Cc1noc(C)c1-c1ccc2nc(-c3cccnc3N)n(-c3ccc(CN4CCC(Nc5ccnc(C#N)n5)CC4)cc3)c2n1]

A mixture of Example 405 (200 mg, 372 μmol), (3,5-dimethylisoxazol-4-yl)boronic acid (58 mg, 410 μmol), K₂PO₄ (158 mg, 745 μmol), PCy₃ (10.4 mg, 37 μmol) and cataCXiumAPdG3 (136 mg, 186 μmol) in DMF (3 mL) was degassed and purged with N₂ three times, and then was stirred at 120° C. for 2 hr under N₂ atmosphere. The reaction mixture was diluted with H₂O (10 mL) and extracted with EtOAc (10 mL×2). The combined organic layers were washed with brine (30 mL), dried over Na₂SO₄, filtered, and concentrated under reduced pressure. After purified by prep-HPLC (column: Waters Daisogel SP ODS RPS 150×25 mm×5 μm; mobile phase: [water (NH₄HCO₃)-ACN]; gradient: 36%-66% B over 10 min), 4-((I-(4-(2-(2-aminopyridin-3-yl)-5-(3,5-dimethylisoxazol-4-yl)-3H-imidazo[4,5-b]pyridin-3-yl)benzyl)piperidin-4-yl)amino)pyrimidine-2-carbonitrile (Example 473, 135.4 mg, yield: 61%) was obtained as an off white powder. MS: m/z=598.4 [M+H]⁺. ¹H NMR (400 MHz, Dimethysulfoxide-d₆) δ 8.28 (d, J=8.4 Hz, 1H), 8.23-7.94 (m, 3H), 7.55 (d, J=8.4 Hz, 1H), 7.50-7.37 (m, 4H), 7.22 (dd, J=8.0, 2.0 Hz, 1H), 7.09 (s, 2H), 6.67 (d, J=6.0 Hz, 1H), 6.40 (dd, J=7.6, 4.8 Hz, 1H), 3.89-3.69 (m, 1H), 3.57 (s, 2H), 2.79 (d, J=10.8 Hz, 2H), 2.52 (s, 3H), 2.31 (s, 3H), 2.17-2.06 (m, 2H), 1.92-1.80 (m, 2H), 1.54-1.41 (m, 2H).

### Example 474: 4-((1-(4-(2-(2-Aminopyridin-3-yl)-5-(1-(difluoromethyl)-1H-pyrazol-4-yl)-3H-imidazo[4,5-b]pyridin-3-yl)benzyl)piperidin-4- yl)amino)pyrimidine-2-carbonitrile

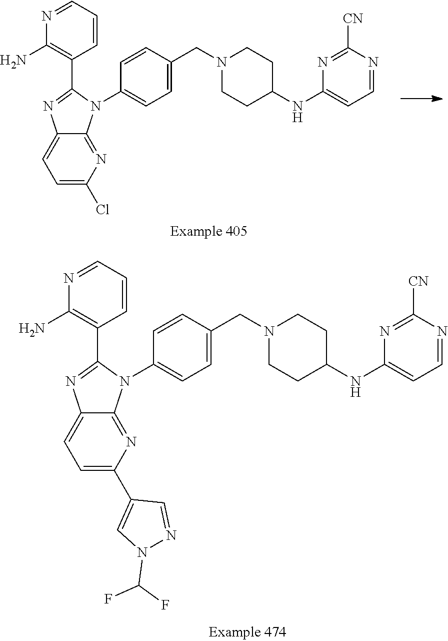 [SMILES: N#Cc1nccc(NC2CCN(Cc3ccc(-n4c(-c5cccnc5N)nc5ccc(Cl)nc54)cc3)CC2)n1 ; N#Cc1nccc(NC2CCN(Cc3ccc(-n4c(-c5cccnc5N)nc5ccc(-c6cnn(C(F)F)c6)nc54)cc3)CC2)n1]

A mixture of Example 405 (200 mg, 372 μmol), (1-(difluoromethyl)-1H-pyrazol-4-yl)boronic acid (66 mg, 410 μmol), K₃PO₄ (158 mg, 745 μmol), PCy₃ (10.4 mg, 37 μmol) and cataCXiumAPdG₃ (136 mg, 186 μmol) in DMF (3 mL) were degassed and purged with N₂ three times, and then was stirred at 120° C. for 2 hr under N₂ atmosphere. The reaction mixture was diluted with H₂O (10 mL) and extracted with EtOAc (10 mL×2). The combined organic layers were washed with brine (30 mL), dried over Na₂SO₄, filtered, and concentrated under reduced pressure. After purified by prep-HPLC (column: Waters Daisogel SP ODS RPS 150×25 mm×5 μm; mobile phase: [water (NH₄HCO₃)-ACN]; gradient: 35%-65% B over 10 min), 4-((1-(4-(2-(2-aminopyridin-3-yl)-5-(1-(difluoromethyl)-1H-pyrazol-4-yl)-3H-imidazo[4,5-b]pyridin-3-yl)benzyl)piperidin-4-yl)amino)pyrimidine-2-carbonitrile (Example 474, 75.7 mg, yield: 33%) was obtained as an off white powder. MS: m/z=619.3 [M+H]⁺. ¹H NMR (400 MHz, Dimethysulfoxide-d₆) δ 8.71 (s, 1H), 8.29 (s, 1H), 8.24 (d, J=8.4 Hz, 1H), 8.13-7.96 (m, 3H), 7.91-7.59 (m, 2H), 7.51-7.40 (m, 4H), 7.13 (dd, J=7.6, 1.6 Hz, 1H), 7.00 (s, 2H), 6.68 (d, J=6.4 Hz, 1H), 6.37 (dd, J=7.6, 4.8 Hz, 1H), 3.92-3.72 (m, 1H), 3.59 (s, 2H), 2.91-2.76 (m, 2H), 2.22-2.06 (m, 2H), 1.97-1.83 (m, 2H), 1.56-1.43 (m, 2H). ¹⁹F NMR (400 MHz, Dimethylsulfoxide-d₆) 5-94.48.

### Example 475: 4-((1-(4-(2-(2-Aminopyridin-3-yl)-5-(2,4-difluorophenyl)-3H-imidazo[4,5-b]pyridin-3-yl)benzyl)piperidin-4-yl)amino)pyrimidine-2-carbonitrile

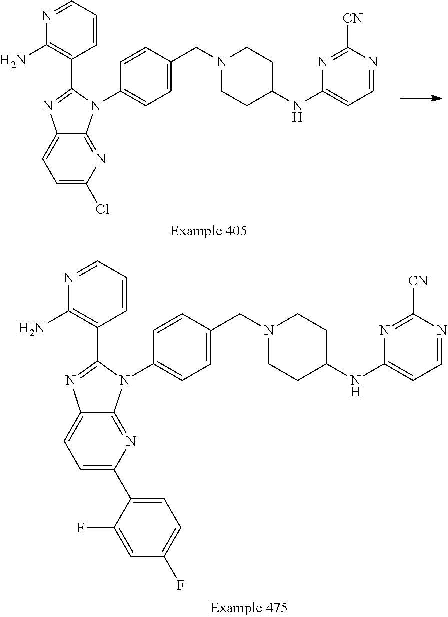 [SMILES: N#Cc1nccc(NC2CCN(Cc3ccc(-n4c(-c5cccnc5N)nc5ccc(Cl)nc54)cc3)CC2)n1 ; N#Cc1nccc(NC2CCN(Cc3ccc(-n4c(-c5cccnc5N)nc5ccc(-c6ccc(F)cc6F)nc54)cc3)CC2)n1]

A mixture of Example 405 (100 mg, 186 μmol), (2,4-difluorophenyl)boronic acid (32 mg, 205 μmol), K₃PO₄ (79 mg, 372 μmol), PCy₃ (5.2 mg, 19 μmol) and cataCXiumAPdG3 (68 mg, 93 μmol) in DMF (3 mL) was degassed and purged with N₂ three times, and then was stirred at 120° C. for 2 hr under N₂ atmosphere. The reaction mixture was diluted with H₂O (10 mL) and extracted with EtOAc (10 mL×2). The combined organic layers were washed with brine (30 mL), dried over Na₂SO₄, filtered and concentrated. After purified by prep-HPLC (column: Waters xbridge 150×25 mm×10 μm, mobile phase: [water (NH₄HCO₃)-ACN]; gradient: 40%-70% B over 11 min), 4-((1-(4-(2-(2-aminopyridin-3-yl)-5-(2,4-difluorophenyl)-3H-imidazo[4,5-b]pyridin-3-yl)benzyl)piperidin-4-yl)amino)pyrimidine-2-carbonitrile (Example 475, 18.9 mg, yield: 17%) was obtained as a light yellow powder. MS: m/z=615.3 [M+H]⁺. ¹H NMR (400 MHz, Methanol-d₄) δ 8.21 (d, J=8.4 Hz, 1H), 8.09-7.88 (m, 3H), 7.84 (dd, J=8.4, 2.0 Hz, 1H), 7.58-7.51 (m, 2H), 7.48-7.42 (m, 2H), 7.33 (dd, J=7.8, 1.6 Hz, 1H), 7.12-6.97 (m, 2H), 6.65-6.44 (m, 1H), 6.48 (dd, J=7.6, 4.8 Hz, 1H), 4.15-3.78 (m, 1H), 3.67 (s, 2H), 3.01-2.91 (m, 2H), 2.33-2.21 (m, 2H), 2.05-1.96 (m, 2H), 1.66-1.55 (m, 2H). ¹⁹F NMR (400 MHz, Dimethylsulfoxide-d₆) δ −111.59, −114.05.

### Example 476: 4-((1-(4-(2-(2-Aminopyridin-3-yl)-5-(6-methoxypyridin-2-yl)-3H-imidazo[4,5-b]pyridin-3-yl)benzyl)piperidin-4-yl)amino)pyrimidine-2-carbonitrile

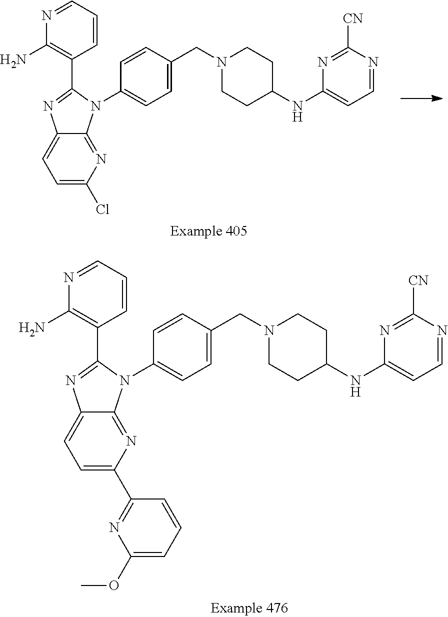 [SMILES: N#Cc1nccc(NC2CCN(Cc3ccc(-n4c(-c5cccnc5N)nc5ccc(Cl)nc54)cc3)CC2)n1 ; COc1cccc(-c2ccc3nc(-c4cccnc4N)n(-c4ccc(CN5CCC(Nc6ccnc(C#N)n6)CC5)cc4)c3n2)n1]

A mixture of Example 405 (300 mg, 559 μmol), (6-methoxypyridin-2-yl)boronic acid (94 mg, 615 μmol), K₃PO₄ (237 mg, 1.12 mmol), PCy₃ (16 mg, 56 μmol) and cataCXiumAPdG3 (203 mg, 279 μmol) in DMF (3 mL) was degassed and purged with N₂ three times, and then was stirred at 120° C. for 2 hr under N₂ atmosphere. The reaction mixture was diluted with H₂O (10 mL) and extracted with EtOAc (10 mL×2). The combined organic layers were washed with brine (30 mL), dried over Na₂SO₄, filtered and concentrated. After purified by prep-HPLC (column: Phenomenex C18 150×25 mm×10 μm; mobile phase: [water (NH₄HCO₃)-ACN]; gradient: 48%-78% B over 11 min), 4-((1-(4-(2-(2-aminopyridin-3-yl)-5-(6-methoxypyridin-2-yl)-3H-imidazo[4,5-b]pyridin-3-yl)benzyl)piperidin-4-yl)amino)pyrimidine-2-carbonitrile (Example 476, 43.7 mg, yield: 13%) was obtained as a yellow powder. MS: m/z=610.5 [M+H]⁺. ¹H NMR (400 MHz, Methanol-d₄) δ 8.56 (d, J=8.4 Hz, 1H), 8.22 (d, J=8.8 Hz, 1H), 8.07-7.94 (m, 2H), 7.88 (d, J=7.6 Hz, 1H), 7.72-7.65 (m, 1H), 7.60-7.53 (m, 2H), 7.50-7.45 (m, 2H), 7.34 (dd, J=7.6, 2.0 Hz, 1H), 6.76 (d, J=8.0 Hz, 1H), 6.65-6.54 (m, 1H), 6.49 (dd, J=7.6, 5.2 Hz, 1H), 4.05 (s, 3H), 4.02-3.89 (m, 1H), 3.68 (s, 2H), 3.02-2.94 (m, 2H), 2.33-2.24 (m, 2H), 2.04-2.00 (m, 2H), 1.67-1.57 (m, 2H).

### Example 477: 4-((1-(4-(2-(2-Aminopyridin-3-yl)-5-(2-methylpyridin-4-yl)-3H-imidazo[4,5-b]pyridin-3-yl)benzyl)piperidin-4-yl)amino)pyrimidine-2-carbonitrile

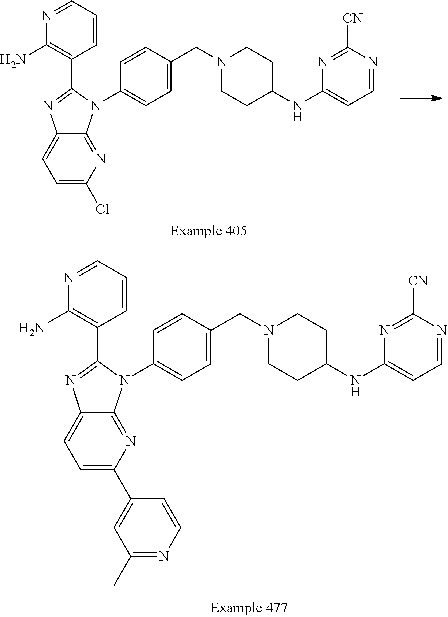 [SMILES: N#Cc1nccc(NC2CCN(Cc3ccc(-n4c(-c5cccnc5N)nc5ccc(Cl)nc54)cc3)CC2)n1 ; Cc1cc(-c2ccc3nc(-c4cccnc4N)n(-c4ccc(CN5CCC(Nc6ccnc(C#N)n6)CC5)cc4)c3n2)ccn1]

A mixture of Example 405 (200 mg, 372 μmol), (2-methylpyridin-4-yl)boronic acid (56 mg, 410 μmol), K₃PO₄ (158 mg, 745 μmol), PCy₃ (10.4 mg, 37 μmol) and cataCXiumAPdG3 (136 mg, 186 μmol) in DMF (3 mL) were degassed and purged with N₂ three times, and then was stirred at 120° C. for 2 hr under N₂ atmosphere. The reaction mixture was diluted with H₂O (10 mL) and extracted with EtOAc (10 mL×2). The combined organic layers were washed with brine (30 mL), dried over Na₂SO₄, filtered, and concentrated under reduced pressure. After purified by prep-HPLC (column: Phenomenex C18 150×25 mm×10 μm; mobile phase: [water (NH₄HCO₃)-ACN]; gradient: 32%-62% B over 11 min), 4-((1-(4-(2-(2-aminopyridin-3-yl)-5-(2-methylpyridin-4-yl)-3H-imidazo[4,5-b]pyridin-3-yl)benzyl)piperidin-4-yl)amino)pyrimidine-2-carbonitrile (Example 477, 46.3 mg, yield: 21%) was obtained as a yellow powder. MS: m/z=594.3 [M+H]⁺. ¹H NMR (400 MHz, Methanol-d₄) δ 8.45 (d, J=5.2 Hz, 1H), 8.31-8.24 (m, 1H), 8.09 (d, J=8.4 Hz, 1H), 8.05-7.98 (m, 2H), 7.95 (s, 1H), 7.90-7.86 (m, 1H), 7.60-7.54 (m, 2H), 7.49-7.38 (m, 2H), 7.34 (dd, J=7.6, 2.0 Hz, 1H), 6.66-6.54 (m, 1H), 6.48 (dd, J=7.6, 4.8 Hz, 1H), 4.09-3.85 (m, 1H), 3.70-3.65 (m, 2H), 3.02-2.95 (m, 2H), 2.58 (s, 3H), 2.33-2.24 (m, 2H), 2.05-1.97 (m, 2H), 1.67-1.58 (m, 2H).

### Example 478: 4-((1-(4-(2-(2-Aminopyridin-3-yl)-5-(5-methylpyridin-3-yl)-3H-imidazo[4,5-b]pyridin-3-yl)benzyl)piperidin-4-yl)amino)pyrimidine-2-carbonitrile

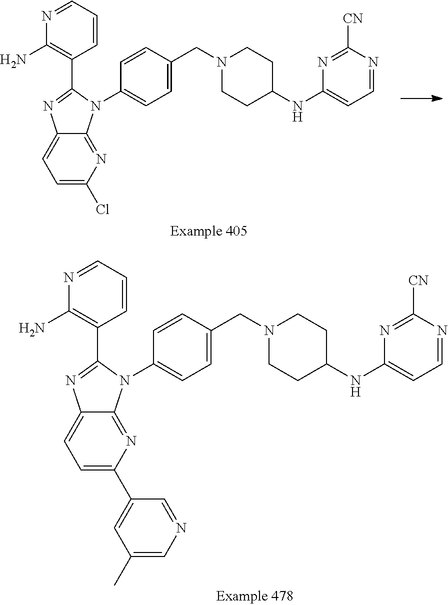 [SMILES: N#Cc1nccc(NC2CCN(Cc3ccc(-n4c(-c5cccnc5N)nc5ccc(Cl)nc54)cc3)CC2)n1 ; Cc1cncc(-c2ccc3nc(-c4cccnc4N)n(-c4ccc(CN5CCC(Nc6ccnc(C#N)n6)CC5)cc4)c3n2)c1]

A mixture of Example 405 (200 mg, 372 μmol), (5-methylpyridin-3-yl)boronic acid (56 mg, 410 μmol), K₃PO₄ (158 mg, 745 μmol), PCy₃ (10.4 mg, 37 μmol) and cataCXiumAPdG3 (136 mg, 186 μmol) in DMF (3 mL) were degassed and purged with N₂ three times, and then was stirred at 120° C. for 2 hr under N₂ atmosphere. The reaction mixture was diluted with H₂O (10 mL) and extracted with EtOAc (10 mL×2). The combined organic layers were washed with brine (30 mL), dried over Na₂SO₄, filtered, and concentrated under reduced pressure. After purified by prep-HPLC (column: Phenomenex C18 150×25 mm×10 μm; mobile phase: [water (NH₄HCO₃)-ACN]; gradient: 32%-62% B over 11 min), 4-((1-(4-(2-(2-aminopyridin-3-yl)-5-(5-methylpyridin-3-yl)-3H-imidazo[4,5-b]pyridin-3-yl)benzyl)piperidin-4-yl)amino)pyrimidine-2-carbonitrile (Example 478, 37.4 mg, yield: 17%) was obtained as a yellow powder. MS: m/z=594.4 [M+H]⁺. ¹H NMR (400 MHz, Methanol-d₄) δ 8.98 (d, J=2.0 Hz, 1H), 8.41-8.33 (m, 1H), 8.29-8.21 (m, 2H), 8.07-7.93 (m, 3H), 7.56 (d, J=8.4 Hz, 2H), 7.45 (d, J=8.4 Hz, 2H), 7.32 (dd, J=7.6, 1.6 Hz, 1H), 6.68-6.53 (m, 1H), 6.47 (dd, J=7.6, 5.2 Hz, 1H), 4.13-3.79 (m, 1H), 3.67 (s, 2H), 3.01-2.92 (m, 2H), 2.42 (s, 3H), 2.27 (t, J=12.0 Hz, 2H), 2.06-1.98 (m, 2H), 1.67-1.55 (m, 2H).

### Example 479: 4-((1-(4-(2-(2-Aminopyridin-3-yl)-5-(1-isopropyl-1H-pyrazol-4-yl)-3H-imidazo[4,5-b]pyridin-3-yl)benzyl)piperidin-4-yl)amino)pyrimidine-2-carbonitrile

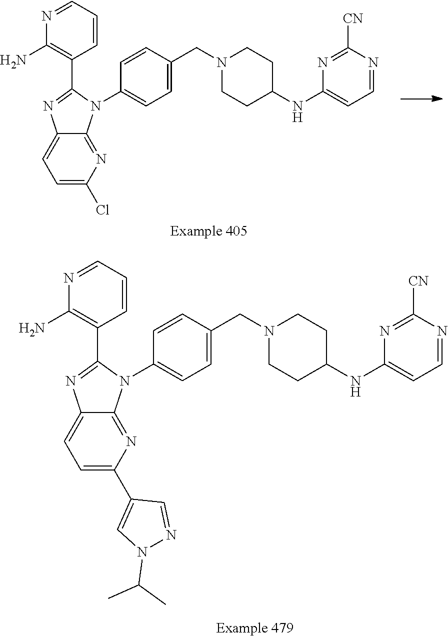 [SMILES: N#Cc1nccc(NC2CCN(Cc3ccc(-n4c(-c5cccnc5N)nc5ccc(Cl)nc54)cc3)CC2)n1 ; CC(C)n1cc(-c2ccc3nc(-c4cccnc4N)n(-c4ccc(CN5CCC(Nc6ccnc(C#N)n6)CC5)cc4)c3n2)cn1]

A mixture of Example 405 (200 mg, 372 μmol), (1-isopropyl-1H-pyrazol-4-yl)boronic acid (63 mg, 410 μmol), K₃PO₄ (158 mg, 745 μmol), PCy₃ (10.4 mg, 37 μmol) and cataCXiumAPdG₃ (135.61 mg, 186.21 μmol) in DMF (3 mL) was degassed and purged with N₂ three times, and then was stirred at 130° C. for 2 hr under N₂ atmosphere. The reaction mixture was diluted with H₂O (10 mL) and extracted with EtOAc (10 mL×2). The combined organic layers were washed with brine (30 mL), dried over Na₂SO₄, filtered, and concentrated under reduced pressure. After purified by prep-HPLC (column: Waters Xbridge BEH C18 150×25 mm×5 μm; mobile phase: [water (NH₄HCO₃)-ACN]; gradient: 32%-62% B over 10 min), 4-((1-(4-(2-(2-aminopyridin-3-yl)-5-(1-isopropyl-1H-pyrazol-4-yl)-3H-imidazo[4,5-b]pyridin-3-yl)benzyl)piperidin-4-yl)amino)pyrimidine-2-carbonitrile (Example 479, 22.8 mg, yield: 10%) was obtained as a yellow solid. MS: m/z=611.3 [M+H]⁺. ¹H NMR (400 MHz, Methanol-d₄) δ 8.15-8.10 (m, 1H), 8.04 (d, J=8.4 Hz, 1H), 8.01-7.96 (m, 1H), 7.96-7.91 (m, 2H), 7.64 (d, J=8.4 Hz, 1H), 7.47 (d, J=8.4 Hz, 2H), 7.36 (d, J=8.4 Hz, 2H), 7.20 (dd, J=7.6, 1.6 Hz, 1H), 6.58 (d, J=5.6 Hz, 1H), 6.41 (dd, J=7.6, 5.2 Hz, 1H), 4.55-4.45 (m, 1H), 4.00-3.85 (m, 1H), 3.59 (s, 2H), 2.91 (d, J=11.6 Hz, 2H), 2.25-2.16 (m, 2H), 1.98 (d, J=10.4 Hz, 2H), 1.61-1.52 (m, 2H), 1.47 (d, J=6.4 Hz, 6H).

### Example 480: 4-((1-(4-(2-(2-Aminopyridin-3-yl)-5-(isothiazol-4-yl)-3H-imidazo[4,5-b]pyridin-3-yl)benzyl)piperidin-4-yl)amino)pyrimidine-2-carbonitrile

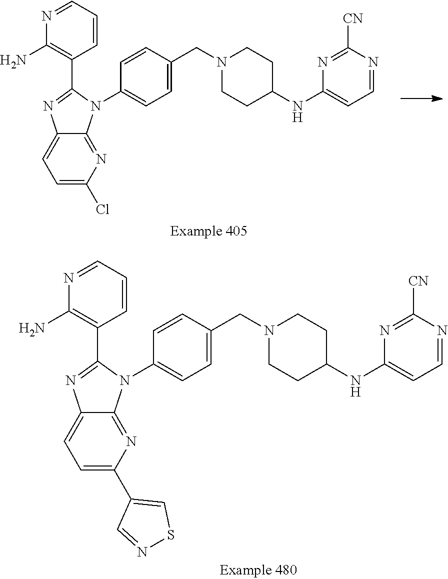 [SMILES: N#Cc1nccc(NC2CCN(Cc3ccc(-n4c(-c5cccnc5N)nc5ccc(Cl)nc54)cc3)CC2)n1 ; N#Cc1nccc(NC2CCN(Cc3ccc(-n4c(-c5cccnc5N)nc5ccc(-c6cnsc6)nc54)cc3)CC2)n1]

A mixture of Example 405 (200 mg, 372 μmol), isothiazol-4-ylboronic acid (58 mg, 447 μmol), K₃PO₄ (158 mg, 745 μmol), PCy₃ (10.4 mg, 37 μmol) and cataCXiumAPdG3 (135.61 mg, 186.21 μmol) in DMF (3 mL) were degassed and purged with N₂ three times, and then was stirred at 120° C. for 2 hr under N₂ atmosphere. The reaction mixture was diluted with H₂O (10 mL) and extracted with EtOAc (10 mL×2). The combined organic layers were washed with brine (30 mL), dried over Na₂SO₄, filtered, and concentrated under reduced pressure. After purified by prep-HPLC (column: Waters Xbridge 150×25 mm×10 μm; mobile phase: [water (NH₄HCO₃)-ACN]; gradient: 28%-58% B over 11 min), 4-((1-(4-(2-(2-aminopyridin-3-yl)-5-(isothiazol-4-yl)-3H-imidazo[4,5-b]pyridin-3-yl)benzyl)piperidin-4-yl)amino)pyrimidine-2-carbonitrile (Example 480, 21.9 mg, yield. 13%) was obtained as a yellow solid. MS: m/z=586.3 [M+H]⁺. ¹H NMR (400 MHz, Dimethysulfoxide-d₆) δ 9.46 (s, 1H), 9.11 (s, 1H), 8.29 (d, J=8.4 Hz, 1H), 8.09-8.04 (m, 2H), 8.02-7.92 (m, 2H), 7.52-7.38 (m, 4H), 7.22-7.11 (m, 1H), 7.08-6.96 (m, 2H), 6.67 (d, J=6.4 Hz, 1H), 6.38 (dd, J=7.6, 4.8 Hz, 1H), 3.92-3.73 (m, 1H), 3.59 (s, 2H), 2.88-2.79 (m, 2H), 2.16 (t, J=10.4 Hz, 2H), 1.93-1.87 (m, 2H), 1.58-1.43 (m, 2H).

### Example 481: 4-((1-(4-(2-(2-Aminopyridin-3-yl)-5-(5-cyano-1-methyl-1H-pyrrol-3-yl)-3H-imidazo[4,5-b]pyridin-3-yl)benzyl)piperidin-4-yl)amino)pyrimidine-2-carbonitrile

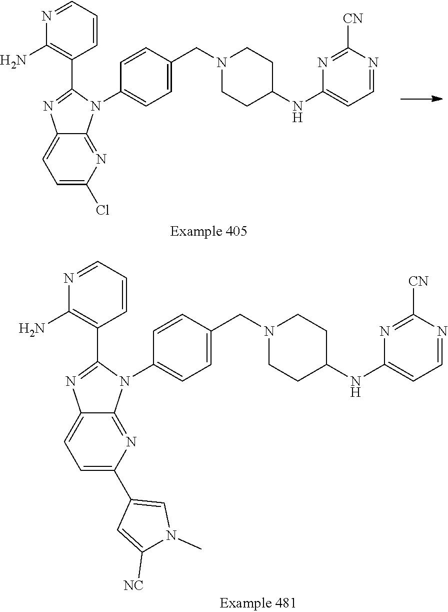 [SMILES: N#Cc1nccc(NC2CCN(Cc3ccc(-n4c(-c5cccnc5N)nc5ccc(Cl)nc54)cc3)CC2)n1 ; Cn1cc(-c2ccc3nc(-c4cccnc4N)n(-c4ccc(CN5CCC(Nc6ccnc(C#N)n6)CC5)cc4)c3n2)cc1C#N]

A mixture of Example 405 (200 mg, 372 μmol), (5-cyano-1-methyl-1H-pyrrol-3-yl)boronic acid (67 mg, 447 μmol), K₃PO₄ (158 mg, 745 μmol), PCy₃ (10.4 mg, 37 μmol) and cataCXiumAPdG3 (135.61 mg, 186.21 μmol) in DMF (5 mL) was degassed and purged with N₂ three times, and then was stirred at 120° C. for 2 hr under N₂ atmosphere. The reaction mixture was diluted with H₂O (10 mL) and extracted with EtOAc (10 mL×2). The combined organic layers were washed with brine (30 mL), dried over Na₂SO₄, filtered and concentrated. After purified by prep-HPLC (column: Waters Xbridge 150×25 mm×10 μm; mobile phase: [water (NH₄HCO₃)-ACN]; gradient: 30%-60% B over 11 min), 4-((1-(4-(2-(2-aminopyridin-3-yl)-5-(5-cyano-1-methyl-1H-pyrrol-3-yl)-3H-imidazo[4,5-b]pyridin-3-yl)benzyl)piperidin-4-yl)amino)pyrimidine-2-carbonitrile (Example 481, 16.3 mg, yield: 7%) was obtained as a yellow solid. MS: m/z=607.3 [M+H]⁺. ¹H NMR (400 MHz, Methanol-d₄) δ 8.08 (d, J=8.4 Hz, 1H), 8.04-7.99 (m, 1H), 7.97 (dd, 1=4.8, 1.2 Hz, 1H), 7.66 (d, J=8.4 Hz, 1H), 7.60-7.56 (m, 1H), 7.54 (d, J=8.0 Hz, 2H), 7.42 (d, J=8.4 Hz, 2H), 7.34-7.25 (m, 2H), 6.60 (d, J=6.4 Hz, 1H), 6.46 (dd, J=7.6, 5.2 Hz, 1H), 4.05-3.90 (m, 1H), 3.81 (s, 3H), 3.63 (s, 2H), 3.01-2.92 (m, 2H), 2.35-2.26 (m, 2H), 2.06-2.00 (m, 2H), 1.67-1.59 (m, 2H).

### Example 482: 4-((1-(4-(2-(2-Aminopyridin-3-yl)-5-(thiazol-4-yl)-3H-imidazo[4,5-b]pyridin-3-yl)benzyl)piperidin-4-yl)amino)pyrimidine-2-carbonitrile

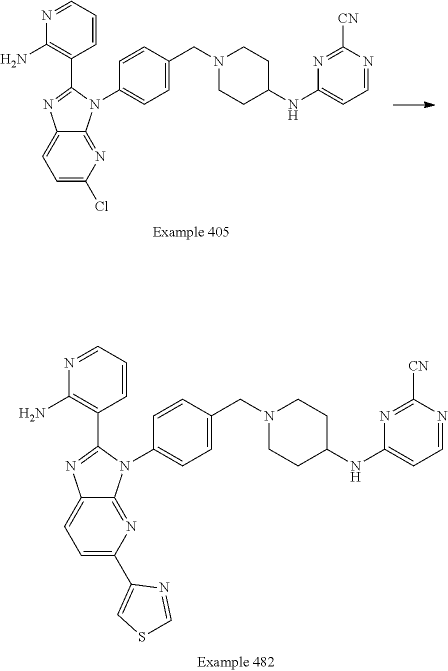 [SMILES: N#Cc1nccc(NC2CCN(Cc3ccc(-n4c(-c5cccnc5N)nc5ccc(Cl)nc54)cc3)CC2)n1 ; N#Cc1nccc(NC2CCN(Cc3ccc(-n4c(-c5cccnc5N)nc5ccc(-c6cscn6)nc54)cc3)CC2)n1]

A mixture of Example 405 (200 mg, 372 μmol), thiazol-4-ylboronic acid (144 mg, 1.12 mmol), Pd(dppf)Cl₂ (27 mg, 37 μmol), Cs₂CO₃ (364 mg, 1.1 mmol) and 1,4-dioxane (4 mL) in H₂O (1 mL) was degassed and purged with N₂ three times, and then was stirred at 100° C. for 3 hr under N₂ atmosphere. The reaction mixture was diluted with H₂O (10 mL) and extracted with EtOAc (10 mL×2). The combined organic layers were washed with brine (30 mL), dried over Na₂SO₄, filtered, and concentrated under reduced pressure. After purified by prep-HPLC (column: Waters Xbridge 150×25 mm×10 μm; mobile phase: [water (NH₄HCO₃)-ACN]; gradient: 35%-65% B over 10 min), 4-((1-(4-(2-(2-aminopyridin-3-yl)-5-(thiazol-4-yl)-3H-imidazo[4,5-b]pyridin-3-yl)benzyl)piperidin-4-yl)amino)pyrimidine-2-carbonitrile (Example 482, 20.1 mg, yield: 9%) was obtained as an off white solid. MS: m/z=608.2 [M+Na]^{+ 1}H NMR (400 MHz, Methanol-d₄) δ 9.05 (s, 1H), 8.25-8.20 (m, 2H), 8.09-8.05 (m, 1H), 8.03-7.94 (m, 2H), 7.57-7.53 (m, 2H), 7.47-7.43 (m, 2H), 7.34-7.28 (m, 1H), 6.60 (d, J=5.6 Hz, 1H), 6.50-6.44 (m, 1H), 4.06-3.88 (m, 1H), 3.66 (s, 2H), 2.97 (d, J=11.2 Hz, 2H), 2.28 (t, J=11.2 Hz, 2H), 2.05-1.99 (m, 2H), 1.67-1.58 (m, 2H).

### Example 483: 4-((1-(4-(2-(2-Aminopyridin-3-yl)-5-(isothiazol-5-yl)-3H-imidazo[4,5-b]pyridin-3-yl)benzyl)piperidin-4-yl)amino)pyrimidine-2-carbonitrile

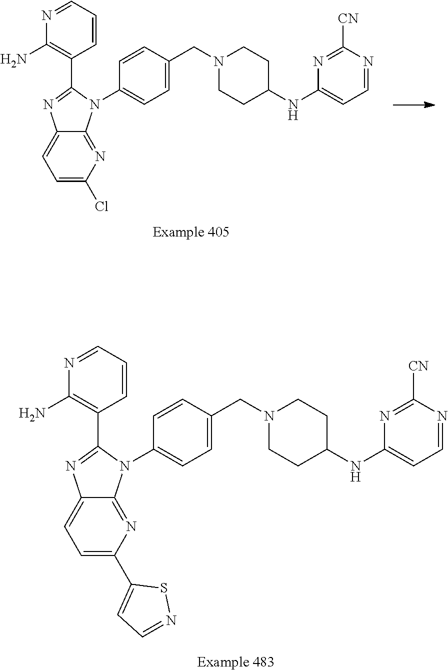 [SMILES: N#Cc1nccc(NC2CCN(Cc3ccc(-n4c(-c5cccnc5N)nc5ccc(Cl)nc54)cc3)CC2)n1 ; N#Cc1nccc(NC2CCN(Cc3ccc(-n4c(-c5cccnc5N)nc5ccc(-c6ccns6)nc54)cc3)CC2)n1]

A mixture of Example 405 (200 mg, 372 μmol), 5-(4,4,5,5-tetramethyl-1,3,2-dioxaborolan-2-yl)isothiazole (94.3 mg, 447 μmol), Pd(dppf)Cl₂ (27 mg, 37 μmol), Cs₂CO₃ (364 mg, 1.1 mmol) and 1,4-dioxane (2 mL) in H₂O (0.5 mL) was degassed and purged with N₂ three times, and then was stirred at 100° C. for 1 hr under N₂ atmosphere. The reaction mixture was diluted with H₂O (10 mL) and extracted with EtOAc (10 mL×2). The combined organic layers were washed with brine (30 mL), dried over Na₂SO₄, filtered, and concentrated under reduced pressure. After purified by prep-HPLC (column: Waters Xbridge 150×25 mm×5 μm; mobile phase: [water (NH₄HCO₃)-ACN]; gradient: 30%-60% B over 10 min), 4-((1-(4-(2-(2-aminopyridin-3-yl)-5-(isothiazol-5-yl)-3H-imidazo[4,5-b]pyridin-3-yl)benzyl)piperidin-4-yl)amino)pyrimidine-2-carbonitrile (Example 483, 21.5 mg, yield: 10%) was obtained as an off white solid. MS: m/z=586.3 [M+H]⁺. ¹H NMR (400 MHz, Methanol-d₄) δ 8.49 (d, J=2.0 Hz, 1H), 8.23 (d, J=8.4 Hz, 1H), 8.11-7.90 (m, 3H), 7.84 (d, J=2.0 Hz, 1H), 7.57 (d, J=8.4 Hz, 2H), 7.44 (d, J=8.4 Hz, 2H), 7.33 (dd, J=7.6, 1.6 Hz, 1H), 6.66-6.54 (m, 1H), 6.48 (dd, J=7.6, 5.2 Hz, 1H), 4.09-3.86 (m, 1H), 3.68 (s, 2H), 2.98 (d, J=11.2 Hz, 2H), 2.35-2.24 (m, 2H), 2.02 (d, J=9.6 Hz, 2H), 1.68-1.55 (m, 2H).

### Example 484: 4-((1-(4-(2-(2-Aminopyridin-3-yl)-5-(1,5-dimethyl-1H-pyrazol-4-yl)-3H-imidazo[4,5-b]pyridin-3-yl)benzyl)piperidin-4-yl)amino)pyrimidine-2-carbonitrile

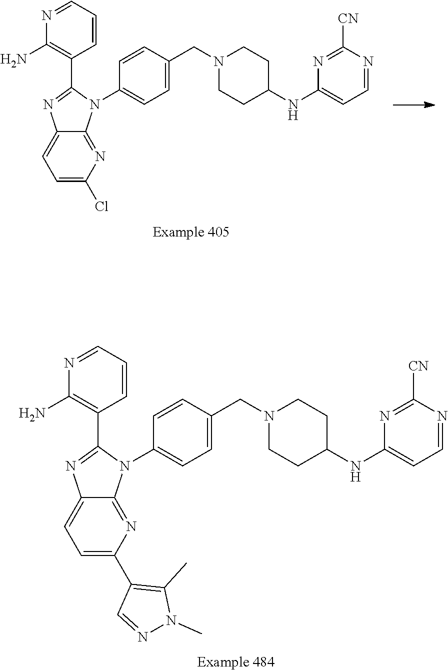 [SMILES: N#Cc1nccc(NC2CCN(Cc3ccc(-n4c(-c5cccnc5N)nc5ccc(Cl)nc54)cc3)CC2)n1 ; Cc1c(-c2ccc3nc(-c4cccnc4N)n(-c4ccc(CN5CCC(Nc6ccnc(C#N)n6)CC5)cc4)c3n2)cnn1C]

A mixture of Example 405 (250 mg, 430 μmol), (1,5-dimethyl-1H-pyrazol-4-yl)boronic acid (72 mg, 516 μmol), Cs₂CO₃ (14 mg, 43 μmol), XphosPdG₄ (370 mg, 430 μmol) in 1,4-dioxane (2 mL) was degassed and purged with N₂ three times, and then was stirred at 90° C. for 16 hr under N₂ atmosphere. The reaction mixture was diluted with H₂O (20 mL) and extracted with EtOAc (20 mL×2). The combined organic layers were washed with brine (30 mL), dried over Na₂SO₄, filtered, and concentrated under reduced pressure. After purified by prep-HPLC (column: Waters Xbridge BEH C18 150×25 mm×5 μm; mobile phase: [water (NH₄HCO₃)-ACN]; gradient: 27%-57% B over 10 min), 4-((1-(4-(2-(2-aminopyridin-3-yl)-5-(1,5-dimethyl-1H-pyrazol-4-yl)-3H-imidazo[4,5-b]pyridin-3-yl)benzyl)piperidin-4-yl)amino)pyrimidine-2-carbonitrile (Example 484, 17 mg, yield: 6%) was obtained as a yellow solid. MS: m/z=597.2 [M+H]⁺. ¹H NMR (400 MHz, Methanol-d₄) δ 8.15-8.08 (m, 1H), 8.05-7.95 (m, 2H), 7.89 (s, 1H), 7.68-7.62 (m, 1H), 7.57-7.50 (m, 2H), 7.46-7.40 (m, 2H), 7.34 (dd, J=7.6, 1.6 Hz, 1H), 6.64-6.55 (m, 1H), 6.47 (dd, J=7.6, 5.2 Hz, 1H), 4.04-3.89 (m, 1H), 3.79 (s, 3H), 3.67 (s, 2H), 2.95 (d, J=11.6 Hz, 2H), 2.52 (s, 3H), 2.26 (t, J=11.2 Hz, 2H), 2.06-1.96 (m, 2H), 1.67-1.51 (m, 2H).

### Example 485: 4-((1-(4-(2-(2-Aminopyridin-3-yl)-5-bromo-3H-imidazo[4,5-b]pyridin-3-yl)benzyl)piperidin-4-yl)amino)pyrimidine-2-carbonitrile

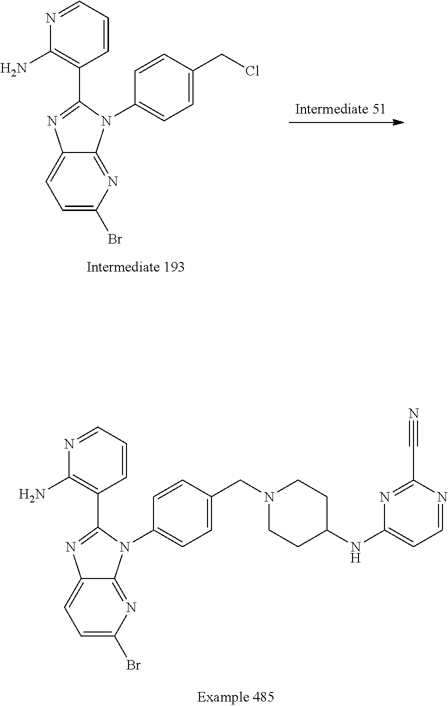 [SMILES: Nc1ncccc1-c1nc2ccc(Br)nc2n1-c1ccc(CCl)cc1 ; N#Cc1nccc(NC2CCN(Cc3ccc(-n4c(-c5cccnc5N)nc5ccc(Br)nc54)cc3)CC2)n1]

To a solution of Intermediate 193 (4.06 g, 9.79 mmol) and Intermediate 51 (2.19 g, 10.8 mmol) in DMF (50 mL) was added DIEA (17.1 mL, 97.94 mmol). The mixture was degassed and purged with N₂ three times and stirred at 60° C. for 2 hr under N₂. The reaction mixture was quenched with H₂O (150 mL) and then extracted with EtOAc (60 mL×3). The combined organic layers were washed with brine (100 mL×3), dried over Na₂SO₄, filtered and concentrated under reduced pressure. After purified by silica gel flash chromatography %61 (MeOH in CH₂Cl₂=1 to 5%) and prep-HPLC (column: Waters xbridge 150×25 mm 10 m; mobile phase: [water (NH₄HCO₃)-ACN]; gradient: 40%-70% B over 11 min), 4-((1-(4-(2-(2-aminopyridin-3-yl)-5-bromo-3H-imidazo[4,5-b]pyridin-3-yl)benzyl)piperidin-4-yl)amino)pyrimidine-2-carbonitrile (Example 485, 2.33 g, 39.6% yield for three steps) was obtained as a yellow solid. MS: m/z=581.2, 583.1 [M+H]⁺. ¹H NMR (400 MHz, Dimethylsulfoxide-d₆) 8.17 (d, J=8.4 Hz, 1H), 8.12-8.02 (m, 2H), 7.99 (dd, J=4.8, 2.0 Hz, 1H), 7.58 (d, J=8.4 Hz, 1H), 7.48-7.45 (m, 2H), 7.41-7.38 (m, 2H), 7.15 (dd, J=7.6, 1.6 Hz, 1H), 6.93 (br s, 2H), 6.68 (d, J=6.0 Hz, 1H), 6.37 (dd, J=7.6, 4.8 Hz, 1H), 3.86-3.76 (m, 1H), 3.57 (s, 2H), 2.85-2.78 (m, 2H), 2.15 (t, J=11.6 Hz, 2H), 1.93-1.84 (m, 2H), 1.54-1.44 (m, 2H).

### Example 486: 4-((1-((4-(2-(2-Aminopyridin-3-yl)-5-bromo-3H-imidazo[4,5-b]pyridin-3-yl)phenyl)methyl-d₂)piperidin-4-yl)amino)pyrimidine-2-carbonitrile

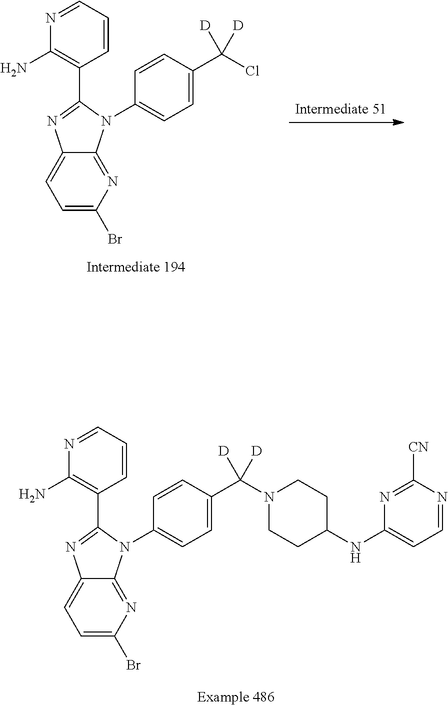 [SMILES: [2H]C([2H])(Cl)c1ccc(-n2c(-c3cccnc3N)nc3ccc(Br)nc32)cc1 ; [2H]C([2H])(c1ccc(-n2c(-c3cccnc3N)nc3ccc(Br)nc32)cc1)N1CCC(Nc2ccnc(C#N)n2)CC1]

To a solution of Intermediate 194 (199 mg, 477 μmol) and Intermediate 51 (97.0 mg, 477 mol) in DMF (3 mL) were added K₂CO₃ (198 mg, 1.43 mmol), NaI (71.5 mg, 477 μmol). The mixture was degassed and purged with N₂ three times and stirred at 25° C. for 16 hr under N₂. The reaction mixture was filtered and concentrated under reduced pressure. After purified by prep-HPLC (column: Phenomenex C18 150×25 mm×10 μm; mobile phase: [water (NH₄HCO₃)-ACN]; gradient: 38%-68% B over 11 min). 4-((1-((4-(2-(2-Aminopyridin-3-yl)-5-bromo-3H-imidazo[4,5-b]pyridin-3-yl)phenyl)methyl-d₂)piperidin-4-yl)amino)pyrimidine-2-carbonitrile (Example 486, 49.2 mg, 16.8% yield for three steps) was obtained as a light yellow solid. MS: m/z=583.2, 585.2 [M+H]⁺. ¹H NMR (400 MHz, Dimethylsulfoxide-d₆) δ 8.17 (d, J=8.4 Hz, 1H), 8.12-8.02 (m, 2H), 7.99 (dd, J=4.8, 1.6 Hz, 1H), 7.58 (d, J=8.4 Hz, 1H), 7.48-7.44 (m, 2H), 7.41-7.37 (m, 2H), 7.15 (dd, J=7.6, 1.6 Hz, 1H), 6.92 (br s, 2H), 6.67 (d, J=6.4 Hz, 1H), 6.37 (dd, J=7.6, 4.8 Hz, 1H), 3.87-3.76 (m, 1H), 2.85-2.78 (m, 2H), 2.19-2.11 (m, 2H), 1.93-1.84 (m, 2H), 1.54-1.44 (m, 2H).

### Example 487: 4-((1-(4-(2-(2-Aminopyridin-3-yl)-5-(4-fluorophenyl)-3H-imidazo[4,5-b]pyridin-3-yl)benzyl)piperidin-4-yl)amino)-1,3,5-triazine-2-carbonitrile

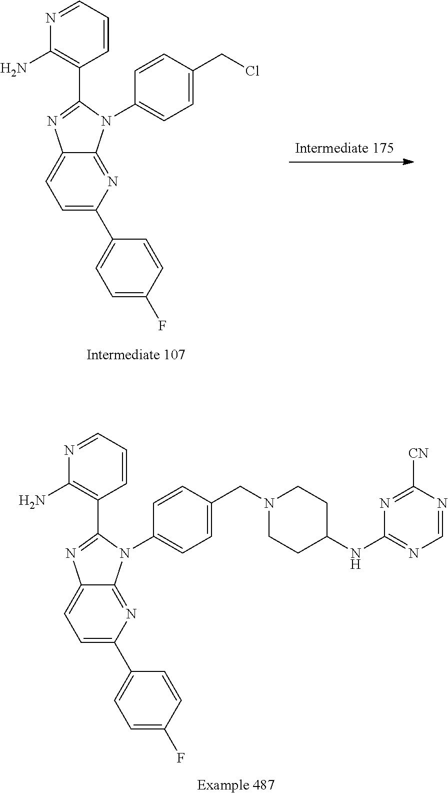 [SMILES: Nc1ncccc1-c1nc2ccc(-c3ccc(F)cc3)nc2n1-c1ccc(CCl)cc1 ; N#Cc1ncnc(NC2CCN(Cc3ccc(-n4c(-c5cccnc5N)nc5ccc(-c6ccc(F)cc6)nc54)cc3)CC2)n1]

To a solution of Intermediate 107 (100 mg, 233 μmol) in DMF (3 mL) were added Intermediate 175 (57 mg, 279 μmol), NaI (35 mg, 233 μmol) and K₂CO₃ (129 mg, 931 μmol) at 25° C. The mixture was stirred at 50° C. for 1 hr. The reaction mixture was poured into H₂O (50 mL) and extracted with CH₂C2 (20 mL×2). The combined organic layers were washed with brine (40 mL), dried over anhydrous Na₂SO₄, filtered and concentrated. After purified by silica gel flash chromatography (Eluent of 0˜5% MeOH in CH₂Cl₂), 4-((1-(4-(2-(2-aminopyridin-3-yl)-5-(4-fluorophenyl)-3H-imidazo[4,5-b]pyridin-3-yl)benzyl)piperidin-4-yl)amino)-1,3,5-triazine-2-carbonitrile (Example 487, 30.0 mg, yield: 20%) was obtained as an yellow solid. MS: m/z=598.3 [M+H]⁺. ¹H NMR (400 MHz, Dimethylsulfoxide-d₆) δ 8.92 (dd, J=7.6, 5.2 Hz, 1H), 8.65 (d, J=29.2 Hz, 1H), 8.27 (d, J=8.4 Hz, 1H), 8.08 (dd, J=8.4, 5.6 Hz, 2H), 8.02-7.95 (m, 2H), 7.51-7.42 (m, 4H), 7.30 (t, J=8.8 Hz, 2H), 7.17-7.13 (m, 1H), 7.03 (s, 2H), 6.41-6.34 (m, 1H), 3.86-3.69 (m, 1H), 3.58 (d, J=4.0 Hz, 2H), 2.91-2.79 (m, 2H), 2.18-2.03 (m, 2H), 1.88-1.78 (m, 2H), 1.66-1.50 (m, 2H). ¹⁹F NMR (400 MHz, Dimethylsulfoxide-d₆) δ −113.58.

### Example 488: 4-((1-((4-(2-(2-Aminopyridin-3-yl)-5-(4-fluorophenyl)-3H-imidazo[4,5-b]pyridin-3-yl)phenyl)methyl-d2)piperidin-4-yl)(methyl)amino)pyrimidine-2-carbonitrile

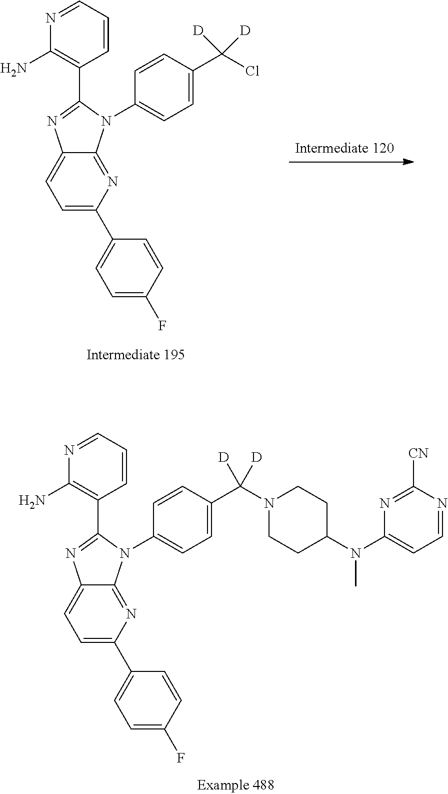 [SMILES: [2H]C([2H])(Cl)c1ccc(-n2c(-c3cccnc3N)nc3ccc(-c4ccc(F)cc4)nc32)cc1 ; [2H]C([2H])(c1ccc(-n2c(-c3cccnc3N)nc3ccc(-c4ccc(F)cc4)nc32)cc1)N1CCC(N(C)c2ccnc(C#N)n2)CC1]

To a solution of Intermediate 195 (100 mg, 232 μmol) in DMF (3 mL) were added Intermediate 120 (60 mg, 278 μmol), NaI (35 mg, 232 μmol) and K₂CO₃ (128 mg, 926 μmol) at 25° C. The mixture was stirred at 50° C. for 1 hr. The reaction mixture was poured into H₂O (50 mL) and extracted with CH₂Cl₂ (20 mL×2). The combined organic layers were washed with brine (40 mL), dried over anhydrous Na₂SO₄, filtered and concentrated. After purified silica gel flash chromatography (Eluent of 0˜5% MeOH in CH₂Cl₂), 4-((1-((4-(2-(2-aminopyridin-3-yl)-5-(4-fluorophenyl)-3H-imidazo[4,5-b]pyridin-3-yl)phenyl)methyl-d₂)piperidin-4-yl)(methyl)amino)pyrimidine-2-carbonitrile (Example 488, 20.9 mg, yield: 14%) was obtained as an off white solid. MS: m/z=613.4 [M+H]⁺. ¹H NMR (400 MHz, Dimethylsulfoxide-d₆) δ 8.21-8.14 (m, 2H), 8.10-8.04 (m, 2H), 7.99 (dd, J=4.8, 1.6 Hz, 1H), 7.92 (d, J=8.4 Hz, 1H), 7.59-7.53 (m, 2H), 7.49-7.44 (m, 2H), 7.32 (dd, J=7.6, 1.6 Hz, 1H), 7.16 (t, J=8.8 Hz, 2H), 6.88-6.78 (m, 1H), 6.48 (dd, J=8.0, 5.2 Hz, 1H), 3.11-3.05 (m, 2H), 2.99 (s, 3H), 2.37-2.23 (m, 2H), 2.07-2.00 (m, 1H), 2.00-1.88 (m, 2H), 1.76-1.66 (m, 2H). ¹⁹F NMR (400 MHz, Dimethylsulfoxide-d₆) δ −115.70.

### Example 489: 4-((1-((4-(2-(2-Aminopyridin-3-yl)-5-(4-fluorophenyl)-3H-imidazo[4,5-b]pyridin-3-yl)phenyl)methyl-d2)piperidin-4-yl)amino)pyrimidine-2-carbonitrile

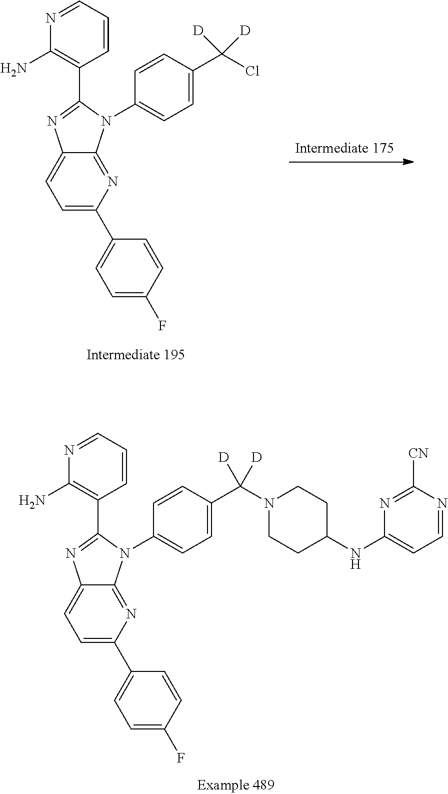 [SMILES: [2H]C([2H])(Cl)c1ccc(-n2c(-c3cccnc3N)nc3ccc(-c4ccc(F)cc4)nc32)cc1 ; [2H]C([2H])(c1ccc(-n2c(-c3cccnc3N)nc3ccc(-c4ccc(F)cc4)nc32)cc1)N1CCC(Nc2ccnc(C#N)n2)CC1]

To a solution of Intermediate 195 (200 mg, 523 μmol) in DMF (3 mL) were added Intermediate 175 (113 mg, 556 μmol), NaI (69 mg, 463 μmol) and K₂CO₃ (256 mg, 1.9 mmol) at 25° C. The mixture was stirred at 50° C. for 1 hr. The reaction mixture was poured into H₂O (50 mL) and extracted with CH₂Cl₂ (20 mL×2). The combined organic layers were washed with brine (40 mL), dried over anhydrous Na₂SO₄, filtered and concentrated. After purified silica gel flash chromatography (Eluent of 0˜5% MeOH in CH₂Cl₂), 4-((1-((4-(2-(2-aminopyridin-3-yl)-5-(4-fluorophenyl)-3H-imidazo[4,5-b]pyridin-3-yl)phenyl))methyl-d₂)piperidin-4-yl)amino)pyrimidine-2-carbonitrile (Example 489, 118.6 mg, yield: 42%) was obtained as an yellow solid. MS: m/z=599.3 [M+H]⁺. ¹H NMR (400 MHz, Dimethylsulfoxide-d₆) δ 8.27 (d, J=8.4 Hz, 1H), 8.11-8.04 (m, 4H), 8.01-7.95 (m, 2H), 7.50-7.43 (m, 4H), 7.29 (t, J=8.8 Hz, 2H), 7.14 (dd, J=7.6, 1.6 Hz, 1H), 7.04 (s, 2H), 6.68 (d, J=6.0 Hz, 1H), 6.37 (dd, J=7.6, 4.8 Hz, 1H), 3.89-3.73 (m, 1H), 2.88-2.78 (m, 2H), 2.21-2.07 (m, 2H), 1.93-1.84 (m, 2H), 1.57-1.43 (m, 2H). ¹⁹F NMR (400 MHz, Dimethylsulfoxide-d₆) δ −113.56.

### Example 490: 4-(7-(4-(2-(2-Aminopyridin-3-yl)-5-(4-fluorophenyl)-3H-imidazo[4,5-b]pyridin-3-yl)benzyl)-2,7-diazaspiro[3.5]nonan-2-yl)pyrimidine-2-carbonitrile

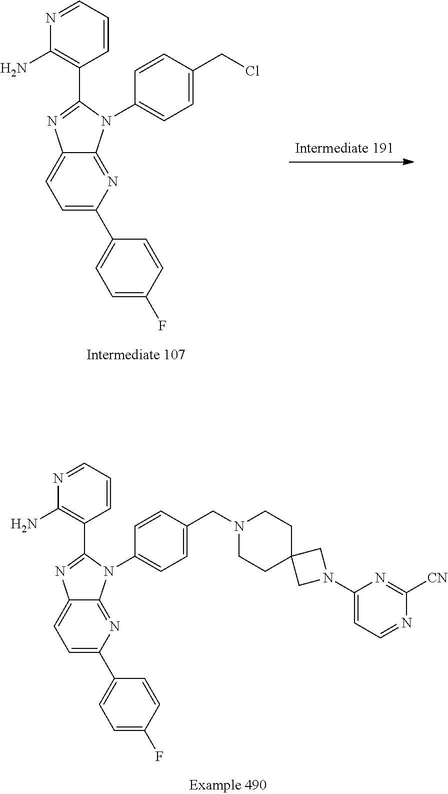 [SMILES: Nc1ncccc1-c1nc2ccc(-c3ccc(F)cc3)nc2n1-c1ccc(CCl)cc1 ; N#Cc1nccc(N2CC3(CCN(Cc4ccc(-n5c(-c6cccnc6N)nc6ccc(-c7ccc(F)cc7)nc65)cc4)CC3)C2)n1]

To a solution of Intermediate 191 (107 mg, 465 μmol, TFA salt) and Intermediate 107 (200 mg, 465 μmol, HCl salt) in DMF (5 mL) were added NaI (35 mg, 233 μmol) and K₂CO₃ (335 mg, 2.43 mmol). The mixture was stirred at 50° C. for 1 hr. After purified by prep-HPLC (column: Waters xbridge 150×25 mm 10 μm; mobile phase: [water (NH₄HCO₃)-ACN]; gradient: 59%-89% B over 10 min), 4-(7-(4-(2-(2-aminopyridin-3-yl)-5-(4-fluorophenyl)-3H-imidazo[4,5-b]pyridin-3-yl)benzyl)-2,7-diazaspiro[3.5]nonan-2-yl)pyrimidine-2-carbonitrile (Example 490, 105.6 mg, yield: 35%) was obtained as a yellow solid. MS: m/z=623.3 [M+H]⁺. ¹H NMR (400 MHz, Dimethylsulfoxide-d₆) δ 8.28 (d, J=8.4 Hz, 1H), 8.22 (d, J=6.0 Hz, 1H), 8.10-8.06 (m, 2H), 8.02-7.97 (m, 2H), 7.50-7.43 (m, 4H), 7.33-7.28 (m, 2H), 7.16 (dd, J=7.6, 2.0 Hz, 1H), 7.03 (s, 2H), 6.64 (d, J=6.0 Hz, 1H), 6.39 (dd, J=7.6, 4.8 Hz, 1H), 3.81 (s, 4H), 3.57 (s, 2H), 2.42-2.31 (m, 4H), 1.81-1.77 (m, 4H).

### Example 491: 4-(2-(4-(2-(2-Aminopyridin-3-yl)-5-(4-fluorophenyl)-3H-imidazo[4,5-b]pyridin-3-yl)benzyl)-2,7-diazaspiro[3.5]nonan-7-yl)pyrimidine-2-carbonitrile

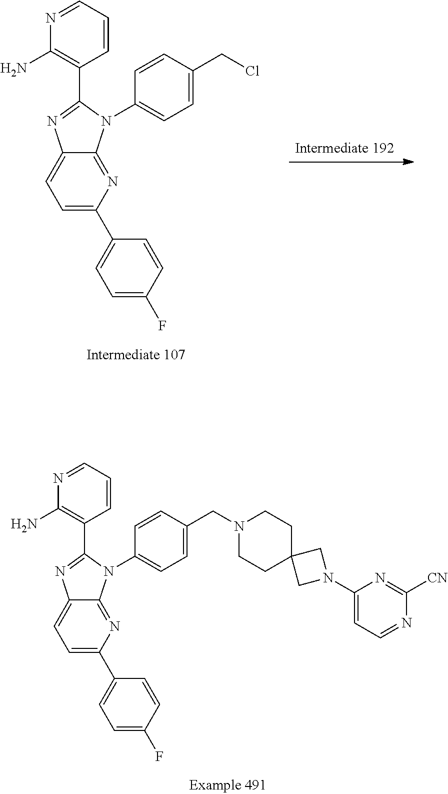 [SMILES: Nc1ncccc1-c1nc2ccc(-c3ccc(F)cc3)nc2n1-c1ccc(CCl)cc1 ; N#Cc1nccc(N2CC3(CCN(Cc4ccc(-n5c(-c6cccnc6N)nc6ccc(-c7ccc(F)cc7)nc65)cc4)CC3)C2)n1]

To a solution of Intermediate 192 (107 mg, 465 μmol, TFA salt) and Intermediate 107 (200 mg, 465 μmol, HCl salt) in DMF (5 mL) were added NaI (35 mg, 233 μmol) and K₂CO₃ (322 mg, 2.33 mmol). The mixture was stirred at 50° C. for 1 hr. After purified by prep-HPLC (column: Waters xbridge 150×25 mm 10 μm; mobile phase: [water (NH₄HCO₃)-ACN]; gradient: 49%-79% B over 10 min), 4-(2-(4-(2-(2-aminopyridin-3-yl)-5-(4-fluorophenyl)-3H-imidazo[4,5-b]pyridin-3-yl)benzyl)-2,7-diazaspiro[3.5]nonan-7-yl)pyrimidine-2-carbonitrile (Example 491, 54.2 mg, yield: 18%) was obtained as a yellow solid. MS: m/z=623.3 [M+H]⁺. ¹H NMR (400 MHz, Dimethylsulfoxide-d₆) δ 8.27 (d, J=8.4 Hz, 1H), 8.22 (d, J=6.4 Hz, 1H), 8.09-8.05 (m, 2H), 8.01-7.96 (m, 2H), 7.45-7.42 (m, 4H), 7.32-7.27 (m, 2H), 7.16 (dd, J=8, 2 Hz, 1H), 7.12 (d, J=6.4 Hz, 1H), 6.99 (s, 2H), 6.39 (dd, J=7.2, 6.4 Hz, 1H), 3.70 (s, 2H), 3.60 (s, 4H), 3.07 (s, 4H), 1.77-1.72 (m, 4H). ¹⁹F NMR (400 MHz, Dimethylsulfoxide-d₆) δ −113.588.

### Example 492: 4-((1-(4-(2-(2-Aminopyridin-3-yl)-5-(methylsulfinyl)-3H-imidazo[4,5-b]pyridin-3-yl)benzyl)piperidin-4-yl)amino)pyrimidine-2-carbonitrile

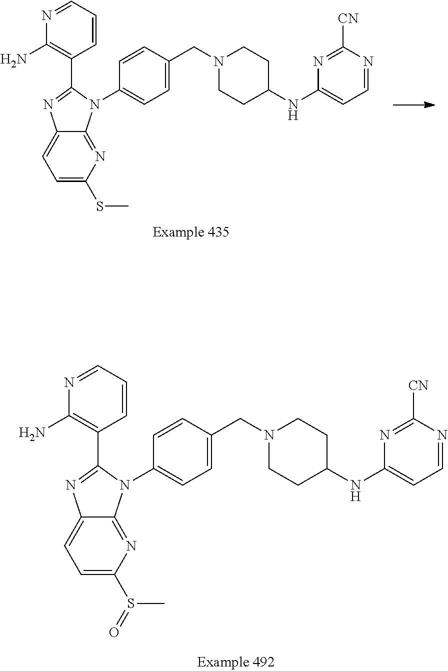 [SMILES: CSc1ccc2nc(-c3cccnc3N)n(-c3ccc(CN4CCC(Nc5ccnc(C#N)n5)CC4)cc3)c2n1 ; CS(=O)c1ccc2nc(-c3cccnc3N)n(-c3ccc(CN4CCC(Nc5ccnc(C#N)n5)CC4)cc3)c2n1]

To a solution of Example 435 (110 mg, 200 μmol) in THF (2 mL) and MeOH (0.5 mL) was added oxone (370 mg, 601 μmol). The mixture was stirred at 25° C. for 2 hr. The reaction mixture was concentrated under reduced pressure. After purified by prep-HPLC (column: Waters xbridge 150*25 mm 10 μm; mobile phase: [water (NH₄HCO₃)-ACN]; gradient: 20%-40% B over 11 min), 4-((1-(4-(2-(2-aminopyridin-3-yl)-5-(methylsulfinyl)-3H-imidazo[4,5-b]pyridin-3-yl)benzyl)piperidin-4-yl)amino)pyrimidine-2-carbonitrile (Example 492, 4.7 mg, yield: 4%) was obtained as light yellow solid. MS: m/z=565.2 [M+H]⁺. ¹H NMR (400 MHz, Methanol-d₄) δ 8.42 (d, J=8.0 Hz, 1H), 8.06-7.96 (m, 3H), 7.54 (d, J=8.0 Hz, 2H), 7.41 (d, J=8.0 Hz, 2H), 7.32 (dd, J=7.6, 1.2 Hz, 1H), 6.63-6.59 (m, 1H), 6.47 (dd, J=7.6, 5.2 Hz, 1H), 4.02-3.91 (m, 1H), 3.66 (s, 2H), 2.98-2.91 (m, 2H), 2.86 (s, 3H), 2.32-2.22 (m, 2H), 2.07-1.96 (m, 2H), 1.61-1.57 (m, 2H).

### Example 493: 4-((1-(4-(2-(2-Aminopyridin-3-yl)-5,7-dimethyl-3H-imidazo[4,5-b]pyridin-3-yl)benzyl)piperidin-4-yl)amino)pyrimidine-2-carbonitrile

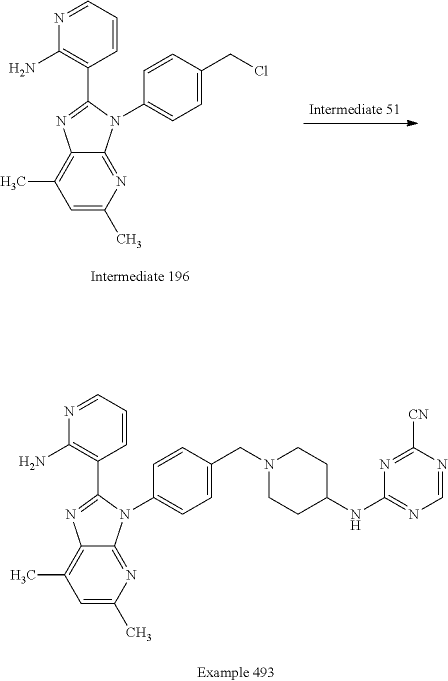 [SMILES: Cc1cc(C)c2nc(-c3cccnc3N)n(-c3ccc(CCl)cc3)c2n1 ; Cc1cc(C)c2nc(-c3cccnc3N)n(-c3ccc(CN4CCC(Nc5ncnc(C#N)n5)CC4)cc3)c2n1]

To a solution of Intermediate 196 (300 mg, 825 μmol) and Intermediate 51 (168 mg, 825 μmol) in DMF (5 mL) were added NaI (61.8 mg, 412 μmol) and K₂CO₃ (570 mg, 4.12 mmol). The mixture was stirred at 50° C. for 1 hr. The reaction mixture was diluted with H₂O (100 mL) and extracted with EtOAc (100 mL×3). The combined organic layers were washed with brine (200 mL), dried over Na₂SO₄, filtered and concentrated under reduced pressure. After purified by silica gel flash chromatography (Eluent of 0˜6% MeOH in CH₂Cl₂), 4-((1-(4-(2-(2-aminopyridin-3-yl)-5,7-dimethyl-3H-imidazo[4,5-b]pyridin-3-yl)benzyl)piperidin-4-yl)amino)pyrimidine-2-carbonitrile (Example 493, 154 mg, yield: 35%) was obtained as a yellow solid. MS: m/z=531.7 [M+H]⁺. ¹H NMR (400 MHz, Dimethylsulfoxide-d₆) δ 8.07 (dd, J=12.4, 6.0 Hz, 2H), 7.96 (dd, J=4.8, 1.6 Hz, 1H), 7.44 (d, J=8.0 Hz, 2H), 7.33 (d, J=8.0 Hz, 2H), 7.09-7.06 (m, 2H), 7.02 (s, 2H), 6.67 (br d, J=6.0 Hz, 1H), 6.34 (dd, J=7.6, 4.8 Hz, 1H), 3.86-3.76 (m, 1H), 3.56 (s, 2H), 2.85-2.80 (m, 2H), 2.61 (s, 3H), 2.47 (s, 3H), 2.18-2.11 (m, 2H), 1.92-1.84 (m, 1H), 1.53-1.44 (m, 2H).

### Example 494: 4-((1-(4-(8-(2-aminopyridin-3-yl)-6-hydroxy-9H-purin-9-yl)benzyl)piperidin-4-yl)amino)pyrimidine-2-carbonitrile

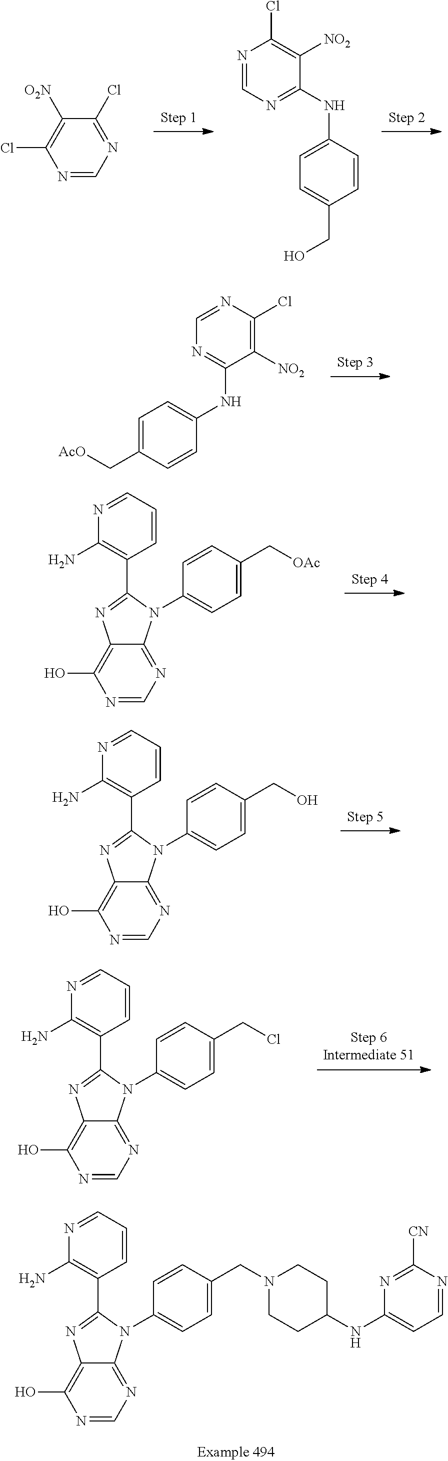

### Step 1: (4-((6-Chloro-5-nitropyrimidin-4-yl)amino)phenyl)methanol

A mixture of 4,6-dichloro-5-nitropyrimidine (10 g, 51.6 mmol), (4-aminophenyl)methanol (5.08 g, 41.2 mmol) in THF (300 mL) was added TEA (4.69 g, 46.4 mmol) at 0° C. The mixture was degassed and purged with N₂ three times, and then was stirred at 0° C. for 0.5 hr under N₂ atmosphere. And then it was stirred at 25° C. for 3 hr. The reaction mixture was diluted with water (400 mL) and extracted with EtOAc (300 mL×3). The combined organic layers were washed with brine (400 mL), dried over Na₂SO₄, filtered and concentrated under reduced pressure. After purified by silica gel flash chromatography (Eluent of 0˜37% EtOAc in petroleum ether), (4-((6-chloro-5-nitropyrimidin-4-yl)amino)phenyl)methanol (8.6 g, yield: 59/a) was obtained as an orange solid. MS: m/z=280.9 [M+H]⁺. ¹H NMR (400 MHz, Dimethylsulfoxide-d₆) δ 10.07 (s, 1H), 8.49 (s, 1H), 7.43 (d, J=8.4 Hz, 2H), 7.32 (d, J=8.4 Hz, 2H), 4.49 (s, 3H).

### Step 2: 4-((6-Chloro-5-nitropyrimidin-4-yl)amino)benzyl acetate

A mixture of (4-((6-chloro-5-nitropyrimidin-4-yl)amino)phenyl)methanol (8 g, 28.5 mmol), DMAP (348 mg, 2.85 mmol) and TEA (8.65 g, 85.5 mmol) in CH₂Cl₂ (200 mL) was added Ac₂O (2.91 g, 28.5 mmol). The mixture was degassed and purged with N₂ three times, and then was stirred at 0° C. for 2 hr under N₂ atmosphere. The reaction mixture was diluted with water (200 mL) and extracted with EtOAc (200 mL×3). The combined organic layers were washed with brine (200 mL), dried over Na₂SO₄, filtered and concentrated under reduced pressure. After purified by silica gel flash chromatography (Eluent of 0˜100% EtOAc in petroleum ether), 4-((6-chloro-5-nitropyrimidin-4-yl)amino)benzyl acetate (1.0 g, yield. 9%) was obtained as a yellow solid. MS: m/z=304.8 [M+H]⁺, ¹H NMR (400 MHz, Chloroform-d) δ 11.28 (s, 1H), 10.91 (s, 1H), 8.10-7.90 (m, 1H), 7.45 (d, J=8.0 Hz, 2H), 7.44 (d, J=8.0 Hz, 2H), 5.13 (s, 2H), 2.05 (s, 3H).

### Step 3: 4-(8-(2-Aminopyridin-3-yl)-6-hydroxy-9H-purin-9-yl)benzyl acetate

A mixture of 4-((6-chloro-5-nitropyrimidin-4-yl)amino)benzyl acetate (343 mg, 3.10 mmol), 2-aminonicotinaldehyde (416 mg, 3.41 mmol) and Na₂S₂O. (2.16 g, 12.4 mmol) in DMSO (100 mL) was degassed and purged with N₂ three times. The mixture was stirred at 100° C. for 16 hr under N₂ atmosphere. The reaction mixture was diluted with H₂O (100 mL) and extracted with EtOAc (100 mL×3). The combined organic layers were washed with brine (200 mL), dried over Na₂SO₄, filtered and concentrated under reduced pressure. After purified by silica gel flash chromatography (Eluent of 0˜80% EtOAc in petroleum ether), 4-(8-(2-aminopyridin-3-yl)-6-hydroxy-9H-purin-9-yl)benzyl acetate (200 mg, yield: 12%) was obtained as a yellow solid. MS: m/z=376.9 [M+H]+, ¹H NMR (400 MHz, Dimethylsulfoxide-d₆) δ 12.67-12.14 (m, 1H), 8.02 (s, 1H), 7.95 (dd, J=4.8, 1.6 Hz, 1H), 7.45 (d, J=8.8 Hz, 2H), 7.40 (d, J=8.0 Hz, 2H), 7.14 (dd, J=7.6, 1.6 Hz, 1H), 6.79 (s, 2H), 6.39 (dd, J=7.6, 4.8 Hz, 1H), 5.14 (s, 2H), 2.10 (s, 3H).

### Step 4: 8-(2-Aminopyridin-3-yl)-9-(4-(hydroxymethyl)phenyl)-9H-purin-6-ol

To a mixture of 4-(8-(2-aminopyridin-3-yl)-6-hydroxy-9H-purin-9-yl)benzyl acetate (150 mg, 399 μmol) in MeOH (10 mL), THF (10 mL) and H₂O (5 mL) was added K₂CO₃ (55.1 mg, 399 μmol). The mixture was stirred at 25° C. for 0.5 hr. The mixture was filtered and concentrated under reduced pressure to give 8-(2-aminopyridin-3-yl)-9-(4-(hydroxymethyl)phenyl)-9H-purin-6-ol (100 mg, yield: 75%) as a yellow solid. MS: m/z=335.0 [M+H]⁺.

### Step 5: 8-(2-Aminopyridin-3-yl)-9-(4-(chloromethyl)phenyl)-9H-purin-6-ol

To a solution of 8-(2-aminopyridin-3-yl)-9-(4-(hydroxymethyl)phenyl)-9H-purin-6-ol (100 mg, 299 μmol) in CH₂Cl₂ (10 mL) was added SOCl₂ (214 mg, 1.79 mmol). The mixture was stirred at 25° C. for 1 hr. The mixture was concentrated under reduced pressure to give 8-(2-aminopyridin-3-yl)-9-(4-(chloromethyl)phenyl)-9H-purin-6-ol (100 mg, yield: 95%) as a yellow solid. MS: m/z=353.1 [M+H]⁺.

### Step 6: 4-((1-(4-(8-(2-Aminopyridin-3-yl)-6-hydroxy-9H-purin-9-yl)benzyl)piperidin-4-yl)amino)pyrimidine-2-carbonitrile

To a solution of 8-(2-aminopyridin-3-yl)-9-(4-(chloromethyl)phenyl)-9H-purin-6-ol (100 mg, 283 μmol) and Intermediate 51 (57.6 mg, 283 μmol) in DMF (5 mL) were added NaI (21.2 mg, 142 μmol) and K₂CO₃ (196 mg, 1.42 mmol). The mixture was stirred at 50° C. for 1 hr. The reaction mixture was filtered and concentrated. After purified by prep-HPLC (column: Waters xbridge 150×25 mm 10 μm; mobile phase: [water (NH₄HCO₃)-ACN]; gradient: 12%-42% B over 10 min), 4-((1-(4-(8-(2-aminopyridin-3-yl)-6-hydroxy-9H-purin-9-yl)benzyl)piperidin-4-yl)amino)pyrimidine-2-carbonitrile (Example 494, 15.3 mg, yield: 9%) was obtained as a yellow solid. MS: m/z=520.1 [M+H]⁺. ¹H NMR (400 MHz, Dimethylsulfoxide-d₆) δ 12.56-12.18 (m, 1H), 8.12-8.02 (m, 3H), 7.94 (dd, J=4.8, 1.6 Hz, 1H), 7.43 (d, J=8.4 Hz, 2H), 7.34 (d, J=8.0 Hz, 2H), 7.06 (dd, J=8.0, 2.0 Hz, 1H), 6.89 (s, 2H), 6.67 (d, J=6.0 Hz, 1H), 6.34 (dd, J=8.0, 4.8 Hz, 1H), 3.86-3.75 (m, 1H), 3.55 (s, 2H), 2.79 (d, J=11.6 Hz, 2H), 2.17-2.09 (m, 2H), 1.91-1.82 (m, 2H), 1.53-1.43 (m, 2H).

### Example 495: 4-((1-(4-(2-(2-Aminopyridin-3-yl)-5-(pyridin-2-yl)-3H-imidazo[4,5-b]pyridin-3-yl)benzyl)piperidin-4-yl)amino)pyrimidine-2-carbonitrile

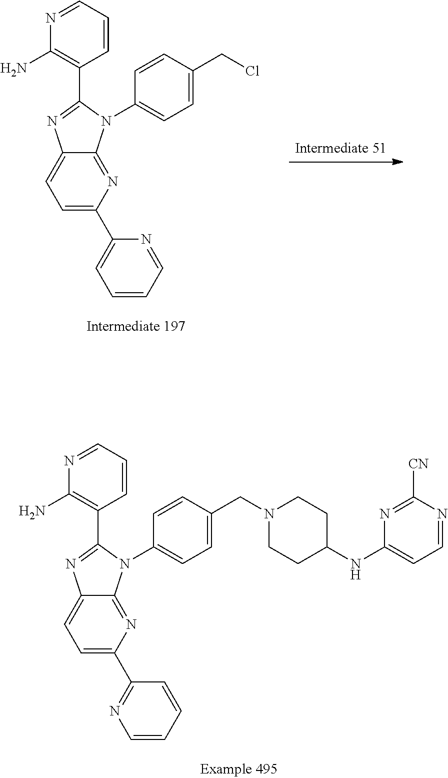 [SMILES: Nc1ncccc1-c1nc2ccc(-c3ccccn3)nc2n1-c1ccc(CCl)cc1 ; N#Cc1nccc(NC2CCN(Cc3ccc(-n4c(-c5cccnc5N)nc5ccc(-c6ccccn6)nc54)cc3)CC2)n1]

To a solution of Intermediate 197 (180 mg, 436 μmol) in DMF (3 mL) were added Intermediate 51 (106 mg, 523 μmol), NaI (65 mg, 436 μmol) and K₂CO₃ (241 mg, 1.7 mmol) at 25° C. The mixture was stirred at 50° C. for 1 hr. The reaction mixture was poured into H₂O (50 mL) and extracted with CH₂Cl₂ (20 mL×2). The combined organic layers were washed with brine (40 mL), dried over anhydrous Na₂SO₄, filtered, and concentrated to dryness. After purified by silica gel flash chromatography (Eluent of 0˜5% MeOH in CH₂Cl₂), 4-((1-(4-(2-(2-aminopyridin-3-yl)-5-(pyridin-2-yl)-3H-imidazo[4,5-b]pyridin-3-yl)benzyl)piperidin-4-yl)amino)pyrimidine-2-carbonitrile (Example 495, 57.2 mg, yield: 22%) was obtained as a yellow solid. MS: m/z=580.3 [M+H]⁺. ¹H NMR (400 MHz, Dimethylsulfoxide-d₆) δ 8.67 (d, J=4.8 Hz, 1H), 8.47 (d, J=8.4 Hz, 1H), 8.31 (d, J=8.4 Hz, 1H), 8.19 (d, J=8.0 Hz, 1H), 8.14-8.03 (m, 2H), 8.03-7.97 (m, 1H), 7.93-7.86 (m, 1H), 7.53-7.44 (m, 4H), 7.40 (dd, J=6.4, 4.8 Hz, 1H), 7.18 (d, J=6.8 Hz, 1H), 7.01 (s, 2H), 6.68 (d, J=5.2 Hz, 1H), 6.39 (dd, J=7.6, 4.8 Hz, 1H), 3.91-3.73 (m, 1H), 3.60 (s, 2H), 2.88-2.78 (m, 2H), 2.24-2.07 (m, 2H), 1.97-1.82 (m, 2H), 1.59-1.42 (m, 2H).

### Example 496: 4-((1-(4-(2-(2-Aminopyridin-3-yl)-5-hydroxy-3H-imidazo[4,5-b]pyridin-3-yl)benzyl)piperidin-4-yl)amino)pyrimidine-2-carbonitrile

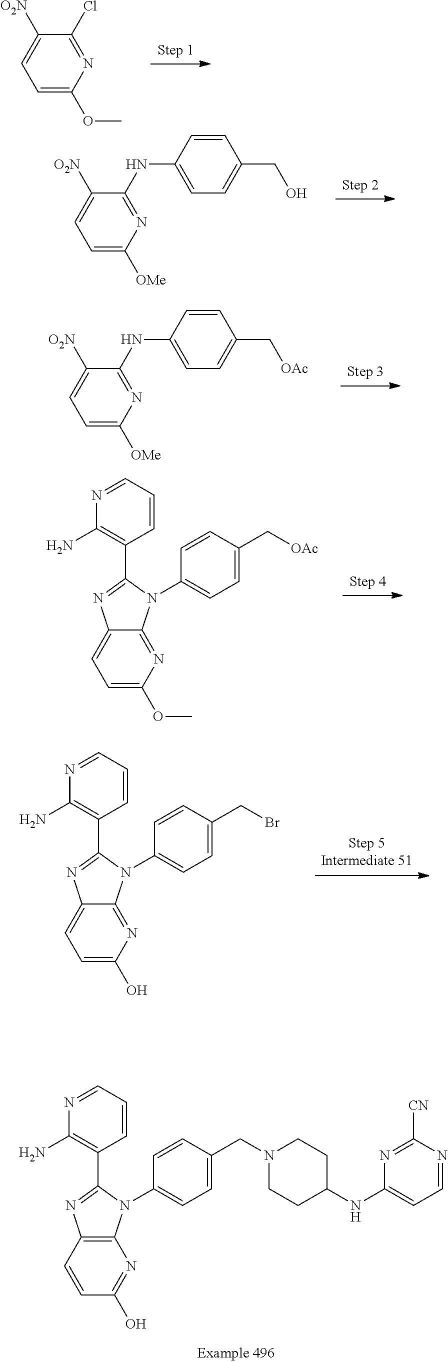

### Step 1: (4-((6-Methoxy-3-nitropyridin-2-yl)amino)phenyl)methanol

To a solution of 2-chloro-6-methoxy-3-nitropyridine (20 g, 106 mmol) and (4-aminophenyl)methanol (13.1 g, 106 mmol) in 1,4-dioxane (220 mL) was added DIEA (41.1 g, 318 mmol). The mixture was stirred at 110° C. for 16 hr. The reaction mixture was diluted with H₂O (500 mL) and extracted with CH₂Cl₂ (500 mL×3). The combined organic layers were washed with brine (400 mL), dried over Na₂SO₄, filtered, and concentrated under reduced pressure. (4-((6-Methoxy-3-nitropyridin-2-yl)amino)phenyl)methanol (29 g, yield: 97%) was obtained as a yellow solid MS: m/z=276.0 [M+H]⁺.

### Step 2: 4-((6-Methoxy-3-nitropyridin-2-yl)amino)benzyl acetate

To a solution of (4-((6-methoxy-3-nitropyridin-2-yl)amino)phenyl)methanol (29 g, 105 mmol) and TEA (32.0 g, 316 mmol) in CH₂Cl₂ (160 mL) were added Ac₂O (10.8 g, 105 mmol) and DMAP (1.29 g, 10.5 mmol). The mixture was stirred at 0° C. for 0.5 hr. The reaction mixture was diluted with H₂O (500 mL) and extracted with CH₂Cl₂ (500 mL×3). The combined organic layers were washed with brine (200 mL), dried over Na₂SO₄, filtered and concentrated under reduced pressure. After purified by silica gel flash chromatography (Eluent of 0˜43% EtOAc in petroleum ether), 4-((6-methoxy-3-nitropyridin-2-yl)amino)benzyl acetate (30 g, yield: 87%) was obtained as a yellow solid. MS: m/z=318.0 [M+H]⁺. ¹H NMR (400 MHz, Chloroform-d) δ 10.66 (s, 1H), 8.42 (d, J=9.2 Hz, 1H), 7.68 (d, J=8.4 Hz, 2H), 7.38 (d, J=8.4 Hz, 2H), 6.25 (d, J=8.8 Hz, 1H), 5.11 (s, 2H), 3.97 (s, 3H), 2.12 (s, 3H).

### Step 3: 4-(2-(2-Aminopyridin-3-yl)-5-methoxy-3H-imidazo[4,5-b]pyridin-3-yl)benzyl acetate

A mixture of 4-((6-methoxy-3-nitropyridin-2-yl)amino)benzyl acetate (15 g, 47.3 mmol), 2-aminonicotinaldehyde (6.93 g, 56.7 mmol), Na₂S₂O. (32.9 g, 189 mmol) in DMSO (100 mL) was degassed and purged with N₂ three times, and then was stirred at 100° C. for 16 hr under N₂ atmosphere. The reaction mixture was diluted with H₂O (1500 mL) and extracted with EtOAc (500 mL×3). The combined organic layers were washed with brine (300 mL), dried over Na₂SO₄, filtered, and concentrated under reduced pressure. The product was triturated with CH₂Cl₂:EtOAc=2:1 (60 mL) and filtered. 4-(2-(2-Aminopyridin-3-yl)-5-methoxy-3H-imidazo[4,5-b]pyridin-3-yl)benzyl acetate (10 g, yield: 50%) was obtained as a brown solid. MS: m/z=390.1 [M+H]⁺. ¹H NMR (400 MHz, Chloroform-d) δ 9.42-8.88 (m, 2H), 8.02 (d, J=8.8 Hz, 1H), 7.74 (d, J=5.6 Hz, 1H), 7.56 (d, J=8.0 Hz, 2H), 7.41-7.33 (m, 3H), 6.83 (d, J=8.8 Hz, 1H), 6.55-6.48 (m, 1H), 5.25 (s, 2H), 3.86 (s, 3H), 2.19 (s, 3H).

### Step 4: 2-(2-Aminopyridin-3-yl)-3-(4-(bromomethyl)phenyl)-3H-imidazo[4,5-b]pyridin-5-ol

To a solution of 4-(2-(2-aminopyridin-3-yl)-5-methoxy-3H-imidazo[4,5-b]pyridin-3-yl)benzyl acetate (500 mg, 1.28 mmol) in CH₂C2 (20 mL) was added BBr3 (3.22 g, 12.8 mmol). The mixture was stirred at 80° C. for 12 hr. The mixture was concentrated under reduced pressure to give 2-(2-aminopyridin-3-yl)-3-(4-(bromomethyl)phenyl)-3H-imidazo[4,5-b]pyridin-5-ol (480 mg, crude) as a brown solid. MS: m/z=395.9, 397.9 [M+H]⁺.

### Step 5: 4-((1-(4-(2-(2-Aminopyridin-3-yl)-5-hydroxy-3H-imidazo[4,5-b]pyridin-3-yl)benzyl)piperidin-4-yl)amino)pyrimidine-2-carbonitrile

To a solution of 2-(2-aminopyridin-3-yl)-3-(4-(bromomethyl)phenyl)-3H-imidazo[4,5-b]pyridin-5-ol (200 mg, 505 μmol) and Intermediate 51 (103 mg, 323 μmol, TFA) in DMF (2 mL) were added K₂CO₃ (349 mg, 2.52 mmol) and NaI (22.7 mg, 151 μmol). The mixture was stirred at 50° C. for 0.5 hr. The reaction mixture was filtered and concentrated. After purified by prep-HPLC(column: Waters xbridge 150×25 mm 10 μm; mobile phase: [water (NH₄HCO₃)-ACN]; gradient: 40%-70% B over 11 min), 4-((1-(4-(2-(2-aminopyridin-3-yl)-5-hydroxy-3H-imidazo[4,5-b]pyridin-3-yl)benzyl)piperidin-4-yl)amino)pyrimidine-2-carbonitrile (Example 496, 23.1 mg delivered, yield: 8.4%) was obtained as a light yellow lyophilized powder. MS: m/z=519.2 [M+H]⁺. ¹H NMR (400 MHz, Dimethylsulfoxide-d₆) δ 11.02-10.64 (m, 1H), 8.10-8.01 (m, 3H), 7.92 (dd, J=4.8, 1.6 Hz, 1H), 7.46-7.42 (m, 2H), 7.37-7.34 (m, 2H), 7.05-6.99 (m, 3H), 6.69-6.61 (m, 2H), 6.31 (dd, J=7.6, 4.8 Hz, 1H), 3.88-3.77 (m, 1H), 3.56 (s, 2H), 2.83-2.78 (m, 2H), 2.17-2.10 (m, 2H), 1.92-1.84 (m, 2H), 1.54-1.44 (m, 2H).

### Example 497: 4-((1-(4-(5-Amino-2-(2-aminopyridin-3-yl)-3H-imidazo[4,5-h]pyridin-3-yl)benzyl)piperidin-4-yl)amino)pyrimidine-2-carbonitrile

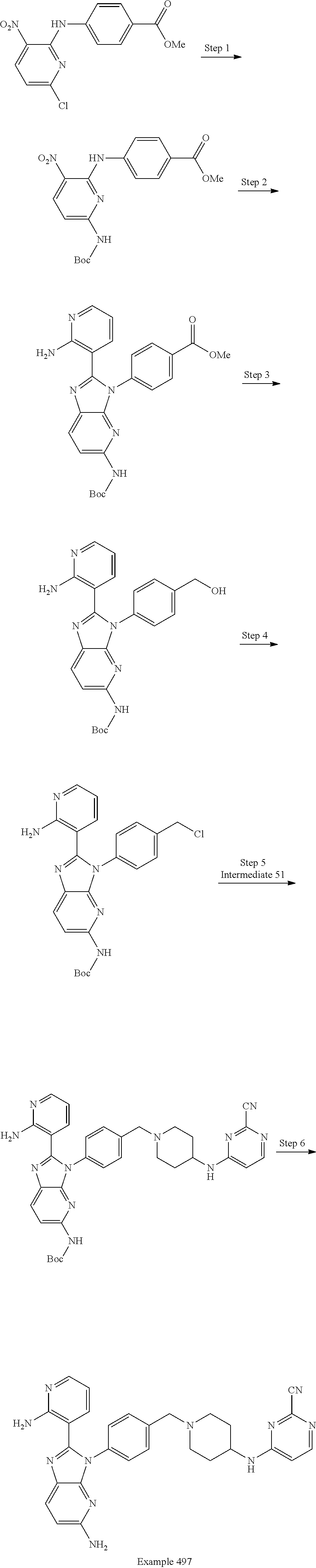

### Step 1: Methyl 4-((6-((tert-butoxycarbonyl)amino)-3-nitropyridin-2-yl)amino)benzoate

A mixture of methyl 4-((6-chloro-3-nitropyridin-2-yl)amino)benzoate (refer to Intermediate 13 for detail procedures, 5 g, 16 mmol), tert-butyl carbamate (1.9 g, 16 mmol), Cs₂CO₃ (10.6 g, 32.5 mmol), XPhos (775 mg, 1.63 mmol) and Pd(OAc)₂ (365 mg, 1.63 mmol) in 1,4-dioxane (60 mL) was degassed and purged with N₂ three times, and then was stirred at 100° C. for 16 hr under N₂ atmosphere. The reaction mixture was diluted with H₂O (100 mL) and extracted with CH₂Cl₂ (100 mL×3). The combined organic layers were washed with brine (50 mL×3), dried over Na₂SO₄, filtered, and concentrated under reduced pressure. Methyl 4-((6-((tert-butoxycarbonyl)amino)-3-nitropyridin-2-yl)amino)benzoate (6 g, yield. 77%) was obtained as a brown solid. MS: m/z=333.0 [M+H−t-Bu]⁺.

### Step 2: Methyl 4-(2-(2-aminopyridin-3-yl)-5-((tert-butoxycarbonyl)amino)-3H-imidazo[4,5-b]pyridin-3-yl)benzoate

To a solution of methyl 4-((6-((tert-butoxycarbonyl)amino)-3-nitropyridin-2-yl)amino)benzoate (2.55 g, 6.57 mmol) in DMSO (20 mL) were added Na₂S₂O₄ (5.38 g, 26.3 mmol, 85% purity) and 2-aminonicotinaldehyde (882 mg, 7.22 mmol). The mixture was stirred at 80° C. for 16 hr. The reaction mixture was quenched with H₂O (25 mL) at 25° C. The mixture was diluted with CH₂Cl₂ (50 mL), washed with H₂O (50 mL×3) and brine (100 mL), dried over Na₂SO₄, filtered, and concentrated under reduced pressure. After purified by silica gel flash chromatography (Eluent of 30%˜50% EtOAc in petroleum ether), methyl 4-(2-(2-aminopyridin-3-yl)-5-((tert-butoxycarbonyl)amino)-3H-imidazo[4,5-b]pyridin-3-yl)benzoate (260 mg, yield: 33%) was obtained as a yellow solid. MS: m/z=461.1 [M+H]⁺. ¹H NMR (400 MHz, Dimethylsulfoxide-d₆) δ 9.71 (s, 1H), 8.15 (d, J=8.8 Hz, 1H), 8.06 (d, J=8.4 Hz, 2H), 7.98 (dd, J=4.8, 1.6 Hz, 1H), 7.88-7.76 (m, 1H), 7.58 (d, J=8.4 Hz, 2H), 7.14 (dd, J=7.6, 1.2 Hz, 1H), 6.83 (s, 2H), 6.41 (dd, J=7.6, 4.8 Hz, 1H), 3.89 (s, 3H), 1.45 (s, 9H).

### Step 3: Tert-butyl (2-(2-aminopyridin-3-yl)-3-(4-(hydroxymethyl)phenyl)-3H-imidazo[4,5-b]pyridin-5-yl)carbamate

To a solution of methyl 4-(2-(2-aminopyridin-3-yl)-5-((tert-butoxycarbonyl)amino)-3H-imidazo[4,5-b]pyridin-3-yl)benzoate (250 mg, 543 μmol) in THF (5 mL) was added LiAlH₄ (41.2 mg, 1.1 mmol) at 0° C. After addition, the resulting mixture was stirred at 25° C. for 1 hr. Then the reaction mixture was filtered. The filtrate was concentrated to give tert-butyl (2-(2-aminopyridin-3-yl)-3-(4-(hydroxymethyl)phenyl)-3H-imidazo[4,5-b]pyridin-5-yl)carbamate (230 mg, yield: 66%) as a yellow solid. MS: m/z=433.1 [M+1]⁺.

### Step 4: Tert-butyl (2-(2-aminopyridin-3-yl)-3-(4-(chloromethyl)phenyl)-3H-imidazo[4,5-b]pyridin-5-yl)carbamate

To a solution of tert-butyl (2-(2-aminopyridin-3-yl)-3-(4-(hydroxymethyl)phenyl)-3H-imidazo[4,5-b]pyridin-5-yl)carbamate (230 mg, 532 μmol) in CH₂Cl₂ (5 mL) was added SOCl₂ (189 mg, 1.6 mmol) at 0° C. The mixture was stirred at 40° C. for 0.5 hr. The reaction mixture was quenched with H₂O (1 mL) at 25° C. and concentrated under reduced pressure. tert-Butyl (2-(2-aminopyridin-3-yl)-3-(4-(chloromethyl)phenyl)-3H-imidazo[4,5-b]pyridin-5-yl)carbamate (260 mg, HCl salt) was obtained as a yellow solid. MS: m/z=451.0 [M+H]⁺.

### Step 5: Tert-butyl (2-(2-aminopyridin-3-yl)-3-(4-((4-((2-cyanopyrimidin-4-yl)amino)piperidin-1-yl)methyl)phenyl)-3H-imidazo[4,5-b]pyridin-5-yl)carbamate

To a solution of tert-butyl (2-(2-aminopyridin-3-yl)-3-(4-(chloromethyl)phenyl)-3H-imidazo[4,5-b]pyridin-5-yl)carbamate (260 mg, 533 μmol, HCl salt) and Intermediate 51 (169 mg, 533 μmol, TFA) in DMF (5 mL) were added K₂CO₃ (369 mg, 2.67 mmol) and NaI (40 mg, 26701). The mixture was stirred at 50° C. for 1 hr. The reaction mixture was quenched with H₂O (25 mL) at 25° C., and then diluted with CH₂Cl₂ (50 mL). The organic layer was washed with H₂O (50 mL×3) and brine (100 mL), dried over Na₂SO₄, filtered and concentrated under reduced pressure. Tert-butyl (2-(2-aminopyridin-3-yl)-3-(4-((4-((2-cyanopyrimidin-4-yl)amino)piperidin-1-yl)methyl)phenyl)-3H-imidazo[4,5-b]pyridin-5-yl)carbamate (300 mg, yield: 67%) was obtained as a yellow solid. MS: m/z=618.1 [M+H]⁺.

### Step 6: 4-((1-(4-(5-Amino-2-(2-aminopyridin-3-yl)-3H-imidazo[4,5-b]pyridin-3-yl)benzyl)piperidin-4-yl)amino)pyrimidine-2-carbonitrile

A mixture of tert-butyl (2-(2-aminopyridin-3-yl)-3-(4-((4-((2-cyanopyrimidin-4-yl)amino)piperidin-1-yl)methyl)phenyl)-3H-imidazo[4,5-b]pyridin-5-yl)carbamate (260 mg, 421 μmol, HCl salt) in 4 M HCl in 1,4-dioxane (3 mL) was stirred at 25° C. for 2 hr. The reaction was concentrated under reduced pressure to give product as a yellow solid. The product was diluted with aqueous NaHCO₃ (10 mL) and extracted with CH₂Cl₂ (10 mL×3). The combined organic layers were washed with brine (20 mL), dried over Na₂SO₄, filtered, and concentrated under reduced pressure. After purified by prep-HPLC (column: Waters xbridge 150×25 mm 10 μm; mobile phase: [water (NH₄HCO₃)-ACN]; gradient: 16%-46% B over 11 min), 4-((1-(4-(5-amino-2-(2-aminopyridin-3-yl)-3H-imidazo[4,5-b]pyridin-3-yl)benzyl)piperidin-4-yl)amino)pyrimidine-2-carbonitrile (Example 497, 21.9 mg, yield: 10%) was obtained as a yellow powder. MS: m/z=518.2 [M+H]⁺. ¹H NMR (400 MHz, Dimethylsulfoxide-d₆) δ 8.06-7.95 (m, 1H), 7.90 (dd, J=4.8, 1.2 Hz, 1H), 7.82 (d, J=8.4 Hz, 1H), 7.53-7.45 (m, 2H), 7.33 (d, J=8.0 Hz, 2H), 7.19 (dd, 1=7.6, 1.2 Hz, 1H), 6.67-6.50 (m, 2H), 6.42 (dd, J=7.6, 4.8 Hz, 1H), 4.08-3.75 (m, 1H), 3.64 (s, 2H), 2.97-2.86 (m, 2H), 2.25 (t, J=11.2 Hz, 2H), 2.05-1.95 (m, 2H), 1.67-1.55 (m, 2H).

### Example 498: 4-((1-(4-(2-(2-Aminopyridin-3-yl)-5-(2-chloro-4-fluorophenyl)-3H-imidazo[4,5-b]pyridin-3-yl)benzyl)piperidin-4-yl)amino)pyrimidine-2-carbonitrile

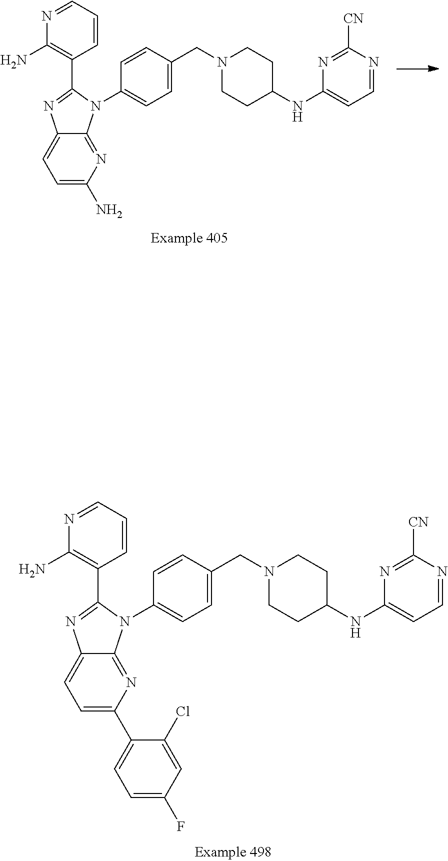 [SMILES: N#Cc1nccc(NC2CCN(Cc3ccc(-n4c(-c5cccnc5N)nc5ccc(N)nc54)cc3)CC2)n1 ; N#Cc1nccc(NC2CCN(Cc3ccc(-n4c(-c5cccnc5N)nc5ccc(-c6ccc(F)cc6Cl)nc54)cc3)CC2)n1]

A mixture of Example 405 (300 mg, 559 μmol), (2-chloro-4-fluorophenyl)boronic acid (107 mg, 615 μmol), K₂CO₃ (232 mg, 1.68 mmol) and Pd(PPh3)4 (65 mg, 56 μmol) in THF (2.5 mL) and H₂O (0.5 mL) was degassed and purged with N₂ three times, and then was stirred at 80° C. for 2 hr under N₂ atmosphere. The reaction mixture was diluted with H₂O (10 mL) and extracted with EtOAc (10 mL×2). The combined organic layers were washed with brine (30 mL), dried over Na₂SO₄, filtered, and concentrated under reduced pressure. After purified by prep-HPLC (column: Phenomenex C18 150×25 mm×10 μm; mobile phase: [water (NH₄HCO₃)-ACN]; gradient: 48%-78% B over 11 min), 4-((1-(4-(2-(2-aminopyridin-3-yl)-5-(2-chloro-4-fluorophenyl)-3H-imidazo[4,5-b]pyridin-3-yl)benzyl)piperidin-4-yl)amino)pyrimidine-2-carbonitrile (Example 498, 129.5 mg, yield: 37%) was obtained as a yellow powder. MS: m/z=631.3 [M+H]⁺. ¹H NMR (400 MHz, Dimethysulfoxide-d₆) δ 8.29 (d, J=8.0 Hz, 1H), 8.13-7.94 (m, 3H), 7.66-7.53 (m, 3H), 7.46-7.38 (m, 4H), 7.36-7.30 (m, 1H), 7.15 (dd, J=7.6, 1.6 Hz, 1H), 6.98 (s, 2H), 6.66 (d, J=6.0 Hz, 1H), 6.38 (dd, J=8.0, 5.2 Hz, 1H), 3.91-3.63 (m, 1H), 3.55 (s, 2H), 2.85-2.75 (m, 2H), 2.17-2.06 (m, 2H), 1.92-1.79 (m, 2H), 1.54-1.41 (m, 2H). ¹⁹F NMR (400 MHz, Dimethylsulfoxide-d₆) δ −111.64.

### Example 499: 4-((1-(4-(2-(2-aminopyridin-3-yl)-5-(4-fluorophenyl)-6-methyl-3H-imidazo[4,5-b]pyridin-3-yl)benzyl)piperidin-4-yl)amino)pyrimidine-2-carbonitrile

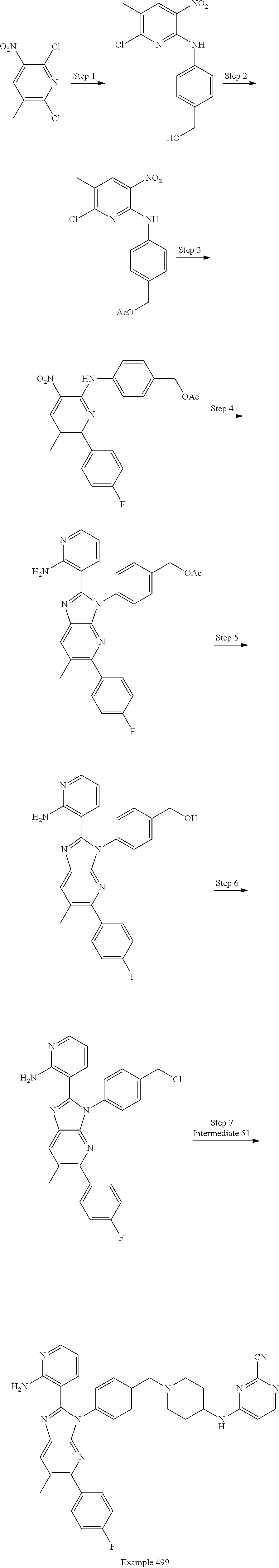

### Step 1: (4-((6-Chloro-5-methyl-3-nitropyridin-2-yl)amino)phenyl)methanol

To a solution of 2,6-dichloro-3-methyl-5-nitropyridine (2 g, 9.7 mmol) and (4-aminophenyl)methanol (1.2 g, 9.7 mmol) in 1,4-dioxane (25 mL) was added DIEA (2.5 g, 19.3 mmol) at 25° C. The mixture was stirred at 70° C. for 12 hr. The reaction mixture was poured into H₂O (100 mL) and extracted with CH₂C2 (50 mL×2). The combined organic layers were washed with brine (40 mL), dried over anhydrous Na₂SO₄, filtered and concentrated. After purified by silica gel flash chromatography (Eluent of 0˜60% EtOAc in petroleum ether), (4-((6-chloro-5-methyl-3-nitropyridin-2-yl)amino)phenyl)methanol (2.3 g, yield: 81%) was obtained as a yellow solid. MS: m/z=293.9 [M+H]⁺. ¹H NMR (400 MHz, Dimethylsulfoxide-d₆) δ 9.96 (s, 1H), 8.55 (d, J=0.6 Hz, 1H), 7.54 (d, J=8.4 Hz, 2H), 7.32 (d, J=8.4 Hz, 2H), 5.16 (t, J=6.4 Hz, 1H), 4.49 (d, J=6.0 Hz, 2H), 2.29 (s, 3H).

### Step 2: 4-((6-Chloro-5-methyl-3-nitropyridin-2-yl)amino)benzyl acetate

To a solution of (4-((6-chloro-5-methyl-3-nitropyridin-2-yl)amino)phenyl)methanol (2.3 g, 7.8 mmol) and acetic anhydride (1.2 g, 11.8 mmol) in CH₂Cl₂ (25 mL) were added TEA (2.4 g, 23.5 mmol) and DMAP (96 g, 783 μmol) at 0° C. The mixture was stirred at 0° C. for 2 hr. The reaction mixture was poured into H₂O (100 mL) and extracted with CH₂Cl₂ (50 mL×2). The combined organic layers were washed with brine (40 mL), dried over anhydrous Na₂SO₄, filtered, and concentrated. After purified by silica gel flash chromatography (Eluent of0˜40% EtOAc in petroleum ether), 4-((6-chloro-5-methyl-3-nitropyridin-2-yl)amino)benzyl acetate (2.5 g, yield: 94%) was obtained as a yellow solid. MS: m/z=335.8 [M+H]⁺. 9.98 (s, 1H), 8.57 (s, 1H), 7.61 (d, J=8.4 Hz, 2H), 7.38 (d, J=8.4 Hz, 2H), 5.06 (s, 2H), 2.30 (s, 3H), 2.07 (s, 3H).

### Step 3: 4-((6-(4-Fluorophenyl)-5-methyl-3-nitropyridin-2-yl)amino)benzyl acetate

To a solution of 4-((6-chloro-5-methyl-3-nitropyridin-2-yl)amino)benzyl acetate (2.2 g, 6.6 mmol) in 1,4-dioxane (30 mL) and H₂O (10 mL) were added Cs₂CO₃ (6.4 g, 20 mmol), (4-fluorophenyl)boronic acid (1 g, 7.2 mmol) and Pd(dppf)Cl₂ (240 mg, 328 μmol) at 25° C. The reaction mixture was degassed and purged with N₂ three times, and then was stirred at 85° C. for 12 hr under N₂. The reaction mixture was poured into H₂O (100 mL) and extracted with CH₂Cl₂ (50 mL×2). The combined organic layers were washed with brine (40 mL), dried over anhydrous Na₂SO₄, filtered and concentrated. After purified by silica gel flash chromatography (Eluent of 0˜60% EtOAc in petroleum ether), 4-((6-(4-fluorophenyl)-5-methyl-3-nitropyridin-2-yl)amino)benzyl acetate (1.8 g, yield: 67%) was obtained as a yellow solid. MS: m/z=396.0 [M+H]⁺. ¹H NMR (400 MHz, Dimethylsulfoxide-d₆) δ 9.95 (s, 1H), 8.52 (s, 1H), 7.74-7.68 (m, 4H), 7.37-7.30 (m, 4H), 5.02 (s, 2H), 2.33 (s, 3H), 2.05 (s, 3H). ¹⁹F NMR (400 MHz, Dimethylsulfoxide-d₆) δ −111.84.

### Step 4: 4-(2-(2-Aminopyridin-3-yl)-5-(4-fluorophenyl)-6-methyl-3H-imidazo[4,5-b]pyridin-3-yl)benzyl acetate

To a solution of 4-((6-(4-fluorophenyl)-5-methyl-3-nitropyridin-2-yl)amino)benzyl acetate (1.8 mg, 4.6 mmol) in DMSO (50 mL) were added 2-aminonicotinaldehyde (667 mg, 5.5 mmol) and Na₂S₂O₄ (3.2 g, 18.2 mmol) at 25° C. The mixture was stirred at 100° C. for 12 hr. The reaction mixture was poured into H₂O (100 mL) and extracted with CH₂Cl₂ (50 mL×2). The combined organic layers were washed with brine (400 mL), dried over anhydrous Na₂SO₄, filtered and concentrated. After purified silica gel by flash chromatography (Eluent of 0˜3% MeOH in CH₂Cl₂), 4-(2-(2-aminopyridin-3-yl)-5-(4-fluorophenyl)-6-methyl-3H-imidazo[4,5-b]pyridin-3-yl)benzyl acetate (1.4 g, yield: 52%) was obtained as a yellow solid. MS: m/z=468.1 [M+H]⁺. ¹H NMR (400 MHz, Dimethylsulfoxide-d₆) δ 8.14 (s, 1H), 7.99 (dd, J=4.8, 2.0 Hz, 1H), 7.56-7.51 (m, 2H), 7.50-7.43 (m, 4H), 7.27 (t, J=8.8 Hz, 2H), 7.18 (dd, J=7.6, 1.6 Hz, 1H), 6.92 (s, 2H), 6.41 (dd, J=7.6, 4.8 Hz, 1H), 5.13 (s, 2H), 2.43 (s, 3H), 2.08 (s, 3H). ¹⁹F NMR (400 MHz, Dimethylsulfoxide-d₆) δ −114.38.

### Step 5: (4-(2-(2-Aminopyridin-3-yl)-5-(4-fluorophenyl)-6-methyl-3H-imidazo[4,5-b]pyridin-3-yl)phenyl)methanol

To a solution of 4-(2-(2-aminopyridin-3-yl)-5-(4-fluorophenyl)-6-methyl-3H-imidazo[4,5-b]pyridin-3-yl)benzyl acetate (1.3 g, 2.8 mmol) in H₂O (1 mL), MeOH (3 mL) and THF (3 mL) was added K₂CO₃ (769 mg, 5.6 μmol) at 25° C. The mixture was stirred at 25° C. for 1 hr. The reaction mixture was poured into H₂O (50 mL) and extracted with CH₂Cl₂ (20 mL×2). The combined organic layers were washed with brine (40 mL), dried over anhydrous Na₂SO₄, filtered and concentrated. (4-(2-(2-Aminopyridin-3-yl)-5-(4-fluorophenyl)-6-methyl-3H-imidazo[4,5-b]pyridin-3-yl)phenyl)methanol (860 mg, yield: 57%) was obtained as a yellow solid. MS: m/z=426.1 [M+H]⁺. ¹H NMR (400 MHz, Dimethylsulfoxide-d₆) δ 8.13 (s, 1H), 7.99 (dd, 1=4.8, 2.0 Hz, 1H), 7.53 (dd, J=8.4, 5.6 Hz, 2H), 7.45-7.36 (m, 4H), 7.26 (t, 0.1=9.2 Hz, 2H), 7.18 (dd, J=7.6, 1.6 Hz, 1H), 7.00 (s, 2H), 6.40 (dd, J=7.85, 4.8 Hz, 1H), 5.31 (t, J=6.0 Hz, 1H), 4.55 (d, J=6.0 Hz, 2H), 2.43 (s, 3H). ¹⁹F NMR (400 MHz, Dimethylsulfoxide-d₆) δ −114.42.

### Step 6: 3-(3-(4-(Chloromethyl)phenyl)-5-(4-fluorophenyl)-6-methyl-3H-imidazo[4,5-b]pyridin-2-yl)pyridin-2-amine

To a solution of (4-(2-(2-aminopyridin-3-yl)-5-(4-fluorophenyl)-6-methyl-3H-imidazo[4,5-b]pyridin-3-yl)phenyl)methanol (200 mg, 470 μmol) in CH₂Cl₂ (5 mL) was added SOCl₂ (280 mg, 2.4 mmol) at 25° C. The mixture was stirred at 40° C. for 0.5 hr. The reaction mixture was concentrated under reduced pressure to give 3-(3-(4-(chloromethyl)phenyl)-5-(4-fluorophenyl)-6-methyl-3H-imidazo[4,5-b]pyridin-2-yl)pyridin-2-amine (200 mg, crude) as a yellow solid, which was used in the next step without further purification. MS: m/z=444.0 [M+H]⁺. ¹H NMR (400 MHz, Dimethylsulfoxide-d₆) δ 8.15 (s, 1H), 8.00 (dd, J=4.8, 1.6 Hz, 1H), 7.60-7.51 (m, 4H), 7.45 (d, J=8.0 Hz, 2H), 7.27 (t, J=8.8 Hz, 2H), 7.22-7.17 (m, 1H), 6.97 (br s, 2H), 6.43 (dd, J=7.2, 4.8 Hz, 1H), 4.82 (s, 2H), 2.44 (s, 3H). ¹⁹F NMR (400 MHz, Dimethylsulfoxide-d₆) δ −114.34.

### Step 7: 4-((1-(4-(2-(2-Aminopyridin-3-yl)-5-(4-fluorophenyl)-6-methyl-3H-imidazo[4,5-b]pyridin-3-yl)benzyl)piperidin-4-yl)amino)pyrimidine-2-carbonitrile

To a solution of 3-(3-(4-(chloromethyl)phenyl)-5-(4-fluorophenyl)-6-methyl-3H-imidazo[4,5-b]pyridin-2-yl)pyridin-2-amine (200 mg, 451 μmol) in DMF (3 mL) were added Intermediate 51 (110 mg, 541 μmol), NaI (34 mg, 225 μmol) and K₂CO₃ (249 mg, 1.8 mmol) at 25° C. The mixture was stirred at 40° C. for 1 hr. The reaction mixture was poured into H₂O (50 mL) and extracted with CH₂Cl₂ (20 mL×2). The combined organic layers were washed with brine (40 mL), dried over anhydrous Na₂SO₄, filtered, and concentrated to dryness. After purified by silica gel flash chromatography (Eluent of 0˜5% MeOH in CH₂Cl₂), 4-((1-(4-(2-(2-aminopyridin-3-yl)-5-(4-fluorophenyl)-6-methyl-3H-imidazo[4,5-b]pyridin-3-yl)benzyl)piperidin-4-yl)amino)pyrimidine-2-carbonitrile (Example 499, 126.3 mg, yield: 45% for two steps) was obtained as a yellow solid. MS: m/z=611.4 [M+H]⁺. ¹H NMR (400 MHz, Dimethylsulfoxide-d₆) δ 8.15 (s, 1H), 8.09 (d, J=6.0 Hz, 1H), 8.04 (d, J=7.2 Hz, 1H), 7.99 (dd, J=4.8, 1.6 Hz, 1H), 7.58-7.51 (m, 2H), 7.46-7.36 (m, 4H), 7.31-7.25 (m, 2H), 7.11 (dd, J=7.6, 1.6 Hz, 1H), 7.03 (s, 2H), 6.67 (d, J=6.0 Hz, 1H), 6.37 (dd, J=7.6, 4.4 Hz, 1H), 3.87-3.73 (m, 1H), 3.55 (s, 2H), 2.85-2.75 (m, 2H), 2.44 (s, 3H), 2.20-2.06 (m, 2H), 1.94-1.80 (m, 2H), 1.55-1.41 (m, 2H). ¹⁹F NMR (400 MHz, Dimethylsulfoxide-d₆) δ −114.39.

### Example 500: 2-(2-Aminopyridin-3-yl)-1-(4-((4-((2-cyanopyrimidin-4-yl)amino)piperidin-1-yl)methyl)phenyl)-1H-benzo[d]imidazole-6-carbonitrile

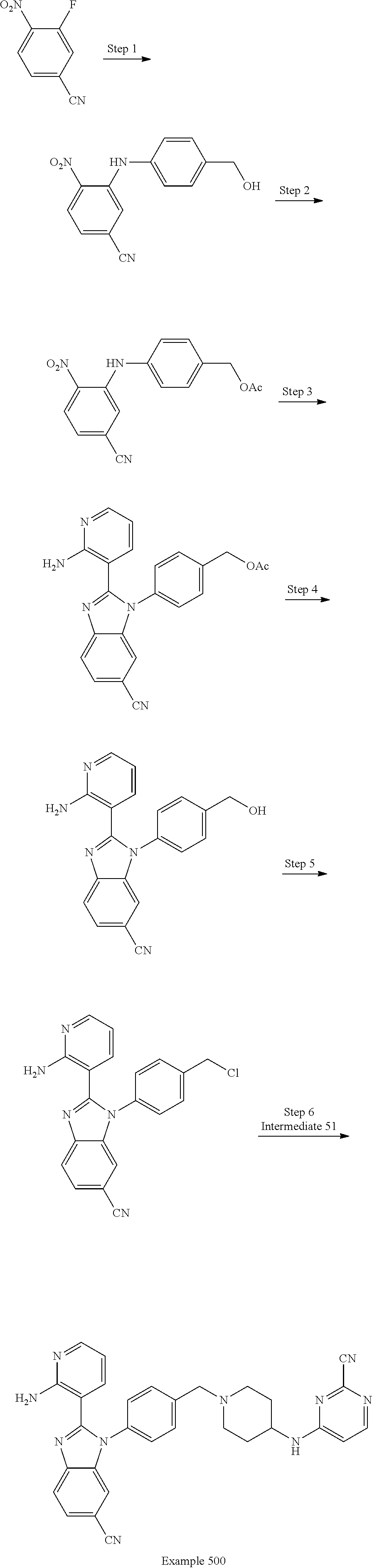

### Step 1: 3-((4-(Hydroxymethyl)phenyl)amino)-4-nitrobenzonitrile

To a solution of 3-fluoro-4-nitrobenzonitrile (6.74 g, 40.6 mmol) and (4-aminophenyl)methanol (5 g, 40.6 mmol) in DMSO (50 mL) was added DIEA (26 g, 203 mmol). The mixture was stirred at 100° C. for 16 hr. The reaction mixture was diluted with H₂O (100 mL) and extracted with CH₂Cl₂ (100 mL×3). The combined organic layers were washed with brine (120 mL), dried over Na₂SO₄, filtered and concentrated under reduced pressure. 3-((4-(Hydroxymethyl)phenyl)amino)-4-nitrobenzonitrile (10.5 g, crude) was obtained as a yellow solid. ¹H NMR (400 MHz, Dimethysulfoxide-d₆) δ 9.47 (s, 1H), 8.23 (d, J=8.8 Hz, 1H), 7.42-7.37 (m, 3H), 7.33-7.28 (m, 2H), 7.20 (dd, J=8.8, 2.0 Hz, 1H), 5.23 (t, J=5.6 Hz, 1H), 4.52 (d, J=6.0 Hz, 2H).

### Step 2: 4-((5-Cyano-2-nitrophenyl)amino)benzyl acetate

To a solution of 3-((4-(hydroxymethyl)phenyl)amino)-4-nitrobenzonitrile (5 g, 18.6 mmol) and TEA (5.64 g, 56 mmol) in CH₂Cl₂ (50 mL) were added Ac₂O (2.84 g, 28 mmol) and DMAP (227 mg, 1.86 mmol). The mixture was stirred at 25° C. for 1 hr. The reaction mixture was diluted with H₂O (300 mL) and extracted with CH₂Cl₂ (200 mL×3). The combined organic layers were washed with brine (300 mL), dried over Na₂SO₄, filtered, and concentrated under reduced pressure. After purified by silica gel flash chromatography (Eluent of 0˜10% EtOAc in petroleum ether), 4-((5-cyano-2-nitrophenyl)amino)benzyl acetate (5.5 g, yield: 95%) was obtained as a red solid. ¹H NMR (400 MHz, Dimethysulfoxide-d₆) δ9.45 (s, 1H), 8.23 (d, J=8.8 Hz, 1H), 7.51 (d, J=1.6 Hz, 1H), 7.45-7.41 (m, 2H), 7.37-7.32 (m, 2H), 7.25 (dd, J=8.8, 1.6 Hz, 1H), 5.08 (s, 2H), 2.08 (s, 3H).

### Step 3: 4-(2-(2-Aminopyridin-3-yl)-6-cyano-1H-benzo[d]imidazol-1-yl)benzyl acetate

A mixture of 4-((5-cyano-2-nitrophenyl)amino)benzyl acetate (3 g, 9.65 mmol), 2-aminonicotinaldehyde (1.29 g, 10.6 mmol), Na₂S₂O₄ (7.9 g, 39 mmol) in DMSO (30 mL) was degassed and purged with N₂ three times, and then was stirred at 100° C. for 16 hr under N₂ atmosphere. The reaction mixture was diluted with H₂O (100 mL) and extracted with CH₂Cl₂ (200 mL×3). The combined organic layers were washed with brine (300 mL), dried over Na₂SO₄, filtered, and concentrated under reduced pressure. After purified by silica gel flash chromatography (Eluent of 10˜50% EtOAc in petroleum ether and 50% CH₂Cl₂ in MeOH), 4-(2-(2-aminopyridin-3-yl)-6-cyano-1H-benzo[d]imidazol-1-yl)benzyl acetate (600 mg, yield: 16%) was obtained as a yellow solid. MS: m/z=384.1 [M+H]⁺. ¹H NMR (400 MHz, Dimethysulfoxide-d₆) δ 8.03-7.94 (m, 2H), 7.76-7.68 (m, 2H), 7.59-7.46 (m, 4H), 7.21 (dd, J=7.6, 1.6 Hz, 1H), 6.91 (s, 2H), 6.42 (dd, J=7.6, 4.8 Hz, 1H), 5.17 (s, 2H), 2.11 (s, 3H).

### Step 4: 2-(2-Aminopyridin-3-yl)-1-(4-(hydroxymethyl)phenyl)-1H-benzo[d]imidazole-6-carbonitrile

To a solution of 4-(2-(2-aminopyridin-3-yl)-6-cyano-1H-benzo[d]imidazol-1-yl)benzyl acetate (570 mg, 1.49 mmol) in THF (2 mL) was added K₂CO₃ (616 mg, 4.46 mmol) in MeOH (2 mL) and H₂O (2 mL). The mixture was stirred at 25° C. for 1 hr. The reaction mixture was diluted with H₂O (20 mL) and extracted with EtOAc (20 mL×3). The combined organic layers were washed with brine (30 mL), dried over Na₂SO₄, filtered and concentrated under reduced pressure. 2-(2-Aminopyridin-3-yl)-1-(4-(hydroxymethyl)phenyl)-1H-benzo[d]imidazole-6-carbonitrile (500 mg, crude) was obtained as a yellow solid. MS: m/z=342.0 [M+H]⁺.

### Step 5: 2-(2-Aminopyridin-3-yl)-1-(4-(chloromethyl)phenyl)-1H-benzo[d]imidazole-6-carbonitrile

To a solution of 2-(2-aminopyridin-3-yl)-1-(4-(hydroxymethyl)phenyl)-1H-benzo[d]imidazole-6-carbonitrile (500 mg, 1.46 mmol) in CH₂Cl₂ (2 mL) was added SOCl₂ (348 mg, 2.93 mmol). The mixture was stirred at 50° C. for 1 hr and then concentrated under reduced pressure. 2-(2-Aminopyridin-3-yl)-1-(4-(chloromethyl)phenyl)-1H-benzo[d]imidazole-6-carbonitrile (530 mg, crude) was obtained as a yellow solid. MS: m/z=360.0 [M+H]⁺.

### Step 6: 2-(2-Aminopyridin-3-yl)-1-(4-((4-((2-cyanopyrimidin-4-yl)amino)piperidin-1-yl)methyl)phenyl)-1H-benzo[d]imidazole-6-carbonitrile

To a solution of 2-(2-aminopyridin-3-yl)-1-(4-(chloromethyl)phenyl)-1H-benzo[d]imidazole-6-carbonitrile (300 mg, 757 μmol) and Intermediate 51 (240 mg, 757 μmol) in DMF (3 mL) were added NaI (56.7 mg, 379 μmol) and K₂CO₃ (418 mg, 3.03 mmol). The mixture was stirred at 25° C. for 16 hr. The reaction mixture was diluted with H₂O (15 mL) and extracted with EtOAc (20 mL×2). The combined organic layers were washed with brine (30 mL), dried over Na₂SO₄, filtered, and concentrated under reduced pressure. After purified by prep-HPLC (column: Phenomenex C18 150×25 mm×10 μm; mobile phase: [water (NH₄HCO₃)-ACN]; gradient: 35%-65% B over 11 min), 2-(2-aminopyridin-3-yl)-1-(4-((4-((2-cyanopyrimidin-4-yl)amino)piperidin-1-yl)methyl)phenyl)-1H-benzo[d]imidazole-6-carbonitrile (Example 500, 157.7 mg, yield: 40%) was obtained as a yellow powder. MS: m/z=527.4 [M+H]⁺. ¹H NMR (400 MHz, Dimethysulfoxide-d₆) δ 8.30-7.90 (m, 4H), 7.78-7.65 (m, 2H), 7.54-7.39 (m, 4H), 7.15 (dd, J=7.6, 1.6 Hz, 1H), 7.01 (s, 2H), 6.84-6.63 (m, 1H), 6.37 (dd, J=7.6, 4.8 Hz, 1H), 3.89-3.74 (m, 1H), 3.59 (s, 2H), 2.83 (d, J=11.2 Hz, 2H), 2.15 (t, J=10.8 Hz, 2H), 1.95-1.80 (m, 2H), 1.56-1.43 (m, 2H).

### Example 501: 2-(2-Aminopyridin-3-yl)-1-(4-((4-((2-cyanopyrimidin-4-yl)amino)piperidin-1-yl)methyl)phenyl)-1H-benzo[d]imidazole-5-carbonitrile

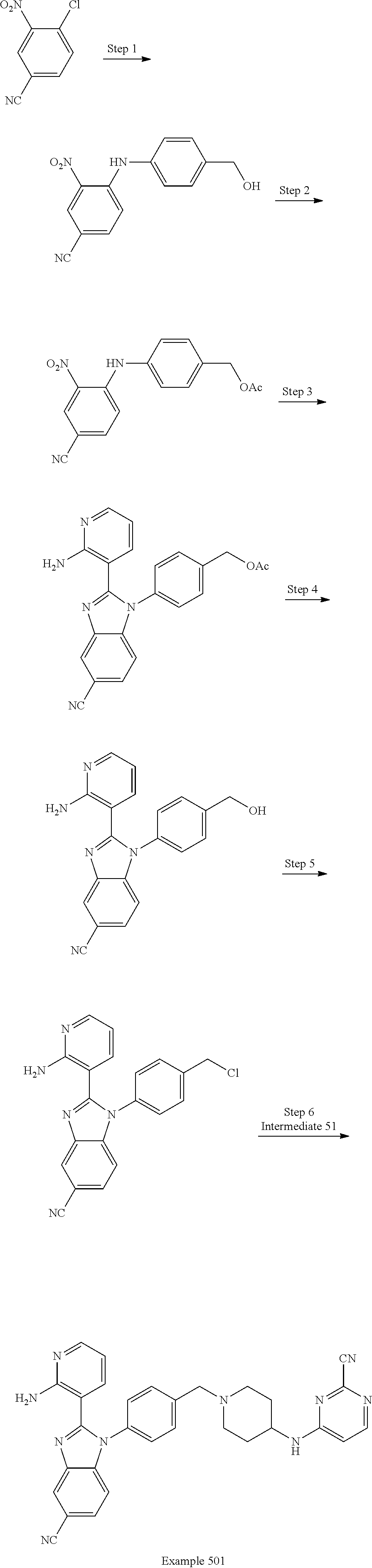

### Step 1: 4-((4-(Hydroxymethyl)phenyl)amino)-3-nitrobenzonitrile

To a solution of 4-chloro-3-nitrobenzonitrile (5 g, 27 mmol) and (4-aminophenyl)methanol (4.05 g, 33 mmol) in DMSO (50 mL) was added DIEA (8.85 g, 68 mmol). The mixture was stirred at 100° C. for 1 hr. The mixture was added H₂O (500 mL), and stirred at 5° C. for 10 min. After filtration, 4-((4-(hydroxymethyl)phenyl)amino)-3-nitrobenzonitrile (7.7 g) was obtained as a yellow solid. ¹H NMR (400 MHz, Dimethysulfoxide-d₆) δ 9.89 (s, 1H), 8.58 (d, J=2.0 Hz, 1H), 7.75 (dd, J=8.8, 2.0 Hz, 1H), 7.42 (d, J=8.4 Hz, 2H), 7.30 (d, J=8.4 Hz, 2H), 7.04 (d, J=9.2 Hz, 1H), 5.27 (t, J=5.6 Hz, 1H), 4.53 (d, J=5.6 Hz, 2H).

### Step 2: 4-((4-Cyano-2-nitrophenyl)amino)benzyl acetate

To a solution of 4-((4-(hydroxymethyl)phenyl)amino)-3-nitrobenzonitrile (7.7 g, 28.6 mmol) and TEA (8.68 g, 86 mmol) in CH₂Cl₂ (70 mL) were added Ac₂O (5.84 g, 57 mmol) and DMAP (349 mg, 2.9 mmol). The mixture was stirred at 25° C. for 1 hr. The reaction mixture was diluted with H₂O (300 mL) and extracted with CH₂Cl₂ (200 mL×3). The combined organic layers were washed with brine (130 mL), dried over Na₂SO₄, filtered and concentrated under reduced pressure. After purified by silica gel flash chromatography (Eluent of 10˜50% EtOAc in petroleum ether), 4-((4-cyano-2-nitrophenyl)amino)benzyl acetate (7.4 g, yield: 83%) was obtained as a yellow solid. ¹H NMR (400 MHz, Dimethysulfoxide-d₆) δ 9.89 (s, 1H), 8.59 (d, J=2.0 Hz, 1H), 7.77 (dd, J=8.8, 2.0 Hz, 1H), 7.47 (d, J=8.4 Hz, 2H), 7.36 (d, J=8.4 Hz, 2H), 7.11 (d, J=8.8 Hz, 1H), 5.10 (s, 2H), 2.08 (s, 3H).

### Step 3: 4-(2-(2-Aminopyridin-3-yl)-5-cyano-1H-benzo[d]imidazol-1-yl)benzyl acetate

A mixture of 4-((4-cyano-2-nitrophenyl)amino)benzyl acetate (7.4 g, 24 mmol), 2-aminonicotinaldehyde (3.19 g, 26 mmol), Na₂S₂O₄ (16.6 g, 95 mmol) in DMSO (200 mL) was degassed and purged with N₂ three times, and then was stirred at 100° C. for 16 hr under N₂ atmosphere. The reaction mixture was diluted with H₂O (300 mL) and extracted with CH₂Cl₂ (200 mL×3). The combined organic layers were washed with brine (150 mL), dried over Na₂SO₄, filtered, and concentrated under reduced pressure. After purified by silica gel flash chromatography (Eluent of 0˜50% EtOAc in petroleum ether), 4-(2-(2-aminopyridin-3-yl)-5-cyano-1H-benzo[d]imidazol-1-yl)benzyl acetate (3.6 g, yield: 33%) was obtained as a yellow solid. MS: m/z=384.0 [M+H]⁺. ¹H NMR (400 MHz, Dimethysulfoxide-d₆) δ 8.37 (s, 1H), 8.00 (dd, J=4.8, 1.6 Hz, 1H), 7.71-7.66 (m, 1H), 7.60-7.53 (m, 2H), 7.50-7.45 (m, 2H), 7.36-7.33 (m, 11H), 7.22 (dd, J=7.6, 2.0 Hz, 11H), 6.88 (br s, 2H), 6.42 (dd, J=7.6, 4.8 Hz, 1H), 5.17 (s, 2H), 2.11 (s, 3H).

### Step 4: 2-(2-Aminopyridin-3-yl)-1-(4-(hydroxymethyl)phenyl)-1H-benzo[d]imidazole-5-carbonitrile

To a solution of 4-(2-(2-aminopyridin-3-yl)-5-cyano-1H-benzo[d]imidazol-1-yl)benzyl acetate (700 mg, 1.83 mmol) in THF (2 mL) were added K₂CO₃ (757 mg, 5.48 mmol), MeOH (2 mL) and H₂O (2 mL). The mixture was stirred at 25° C. for 1 hr. The reaction mixture was diluted with H₂O (20 mL) and extracted with EtOAc (20 mL×3). The combined organic layers were washed with brine (30 mL), dried over Na₂SO₄, filtered and concentrated under reduced pressure. 2-(2-Aminopyridin-3-yl)-1-(4-(hydroxymethyl)phenyl)-1H-benzo[d]imidazole-5-carbonitrile (500 mg, crude) was obtained as a yellow solid. MS: m/z=342.0 [M+H]⁺.

### Step 5: 2-(2-Aminopyridin-3-yl)-1-(4-(chloromethyl)phenyl)-1H-benzo[d]imidazole-5-carbonitrile

To a solution of 2-(2-aminopyridin-3-yl)-1-(4-(hydroxymethyl)phenyl)-1H-benzo[d]imidazole-5-carbonitrile (500 mg, 1.46 mmol) in CH₂Cl₂ (2 mL) was added SOCl₂ (348 mg, 2.93 mmol). The mixture was stirred at 50° C. for 1 hr. The mixture was concentrated under reduced pressure to give 2-(2-aminopyridin-3-yl)-1-(4-(chloromethyl)phenyl)-1H-benzo[d]imidazole-5-carbonitrile (500 mg, crude) as a yellow solid. MS: m/z=359.9 [M+H]⁺.

### Step 6: 2-(2-Aminopyridin-3-yl)-1-(4-((4-((2-cyanopyrimidin-4-yl)amino)piperidin-1-yl)methyl)phenyl)-1H-benzo[d]imidazole-5-carbonitrile

To a solution of 2-(2-aminopyridin-3-yl)-1-(4-(chloromethyl)phenyl)-1H-benzo[d]imidazole-5-carbonitrile (200 mg, 556 μmol) and Intermediate 51 (212 mg, 667 μmol) in DMF (3 mL) were added NaI (83.3 mg, 556 μmol) and K₂CO₃ (230 mg, 1.67 mmol). The mixture was stirred at 50° C. for 1 hr. The reaction mixture was diluted with H₂O (15 mL) and extracted with EtOAc (20 mL×2). The combined organic layers were washed with brine (30 mL), dried over Na₂SO₄, filtered, and concentrated under reduced pressure. After purified by prep-HPLC (column: Phenomenex C18 150×25 mm×10 μm; mobile phase: [water (NH₄HCO₃)-ACN]; gradient: 35%-55% B over 11 min), 2-(2-aminopyridin-3-yl)-1-(4-((4-((2-cyanopyrimidin-4-yl)amino)piperidin-1-yl)methyl)phenyl)-1H-benzo[d]imidazole-5-carbonitrile (Example 501, 30.4 mg, yield: 10%) was obtained as an off white solid. MS: m/z=527.1 [M+H]⁺. ¹H NMR (400 MHz, Methanol-d₄) δ 8.22 (d, J=1.2 Hz, 1H), 8.08-7.95 (m, 2H), 7.67-7.58 (m, 3H), 7.45-7.38 (m, 3H), 7.30 (dd, 1=7.6, 1.6 Hz, 1H), 6.62 (d, J=5.2 Hz, 1H), 6.46 (dd, J=7.6, 5.2 Hz, 1H), 4.04-3.88 (m, 1H), 3.68 (s, 2H), 2.96 (d, J=11.6 Hz, 2H), 2.28 (t, J=10.8 Hz, 2H), 2.07-1.99 (m, 2H), 1.68-1.57 (m, 2H).

### Example 502: 4-((1-((4-(2-(2-Aminopyridin-3-yl)-3H-imidazo[4,5-b]pyridin-3-yl)phenyl)methyl-d2)piperidin-4-yl)(methyl-d3)amino)pyrimidine-2-carbonitrile

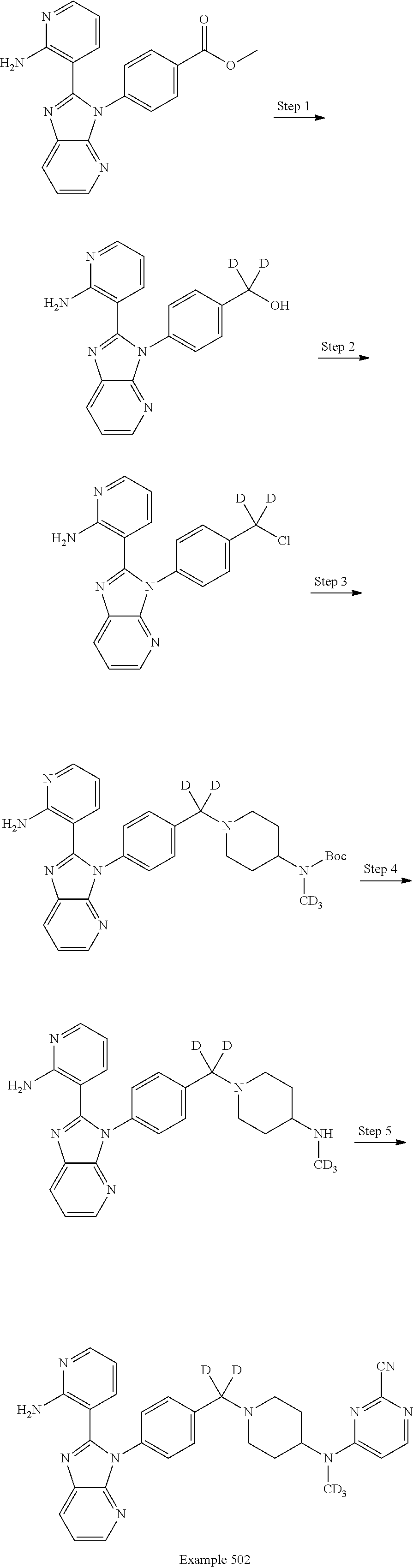

### Step 1: (4-(2-(2-Aminopyridin-3-yl)-3H-imidazo[4,5-b]pyridin-3-yl)phenyl)methan-d₂-ol

To a mixture of methyl 4-(2-(2-aminopyridin-3-yl)-3H-imidazo[4,5-b]pyridin-3-yl)benzoate (300 mg, 869 μmol) in THF (5 mL) was added LiAlD₄ (42.9 mg, 1.13 mmol). The mixture was purged with N₂ three times and stirred at 25° C. for 1 hr under N₂ atmosphere. The reaction mixture was filtered and concentrated under reduced pressure. (4-(2-(2-Amninopyridin-3-yl)-3H-imidazo[4,5-b]pyridin-3-yl)phenyl)methan-d₂-ol (270 mg, yield: 97.3%) was obtained as a pink solid. MS: m/z=319.9 [M+H]⁺.

### Step 2: 3-(3-(4-(Chloromethyl-d₂)phenyl)-3H-imidazo[4,5-b]pyridin-2-yl)pyridin-2-amine

To a solution of (4-(2-(2-aminopyridin-3-yl)-3H-imidazo[4,5-b]pyridin-3-yl)phenyl)methan-d₂-ol (270 mg, 845 μmol) in CH₂Cl₂ (5 mL) was added SOCl₂ (101 mg, 845 μmol). The mixture was stirred at 40° C. for 1 hr. The reaction mixture was quenched with H₂O (0.5 mL) at 0° C., filtered and concentrated under reduced pressure. 3-(3-(4-(Chloromethyl-d₂)phenyl)-3H-imidazo[4,5-b]pyridin-2-yl)pyridin-2-amine (300 mg, HCl salt, yield: 94.8%) was obtained as a pink solid. MS: m/z=338.0 [M+H]⁺.

### Step 3: Tert-butyl (1-((4-(2-(2-aminopyridin-3-yl)-3H-imidazo[4,5-b]pyridin-3-yl)phenyl)methyl-d₂)piperidin-4-yl)(methyl-d₃)carbamate

To a solution of 3-(3-(4-(chloromethyl-d₂)phenyl)-3H-imidazo[4,5-b]pyridin-2-yl)pyridin-2-amine (300 mg, 802 μmol, HCl salt), tert-butyl (methyl-d₃)(piperidin-4-yl)carbamate (174 mg, 802 μmol) in DMF (5 mL) were added NaI (24.0 mg, 160 μmol) and K₂CO₃ (332 mg, 2.40 mmol). The mixture was stirred at 50° C. for 1 hr. The mixture was diluted with H₂O (20 mL) and extracted with CH₂Cl₂ (30 mL×3). The combined organic layers were washed with brine (30 mL×4), dried over Na₂SO₄, filtered and concentrated under reduced pressure. After purified by silica gel flash chromatography (Eluent of 3˜8% CH₂Cl₂ in MeOH), tert-butyl (1-((4-(2-(2-aminopyridin-3-yl)-3H-imidazo[4,5-b]pyridin-3-yl)phenyl)methyl-d₂)piperidin-4-yl)(methyl-d3)carbamate (340 mg, yield: 67.1%) was obtained as a purple solid. MS: m/z=519.2 [M+H]⁺. ¹H NMR (400 MHz, Chloroform-d) δ 8.46-8.36 (m, 1H), 8.13-8.04 (m, 2H), 7.65-7.48 (m, 2H), 7.38-7.30 (m, 3H), 7.10-7.04 (m, 1H), 6.65-6.56 (m, 2H), 6.38-6.33 (m, 1H), 3.09-2.99 (m, 1H), 2.34-2.08 (m, 2H), 1.94-1.71 (m, 2H), 1.71-1.62 (m, 4H), 1.48 (s, 9H).

### Step 4: 3-(3-(4-((4-((Methyl-d3)amino)piperidin-1-yl)methyl-d2)phenyl)-3H-imidazo[4,5-b]pyridin-2-yl)pyridin-2-amine

To a solution of tert-butyl (1-((4-(2-(2-aminopyridin-3-yl)-3H-imidazo[4,5-b]pyridin-3-yl)phenyl)methyl-d₂)piperidin-4-yl)(methyl-d3)carbamate (340 mg, 656 μmol) in MeOH (1 mL) was added HCl in dioxane (4 M, 163.88 μL). The mixture was stirred at 25° C. for 1 hr. The reaction mixture was filtered and concentrated under reduced pressure. 3-(3-(4-((4-((Methyl-d3)amino)piperidin-1-yl)methyl-d₂)phenyl)-3H-imidazo[4,5-b]pyridin-2-yl)pyridin-2-amine (280 mg, yield: 93.8%) was obtained as a pink solid. MS: m/z=419.3 [M+H]⁺. ¹H NMR (400 MHz, Methanol-d₄) δ 8.46 (dd, J=4.8, 1.2 Hz, 1H), 8.34 (dd, J=8.0, 1.2 Hz, 1H), 8.02 (dd, J=6.0, 1.2 Hz, 1H), 7.90-7.85 (m, 3H), 7.66 (d, J=8.4 Hz, 2H), 7.54 (dd, J=8.0, 4.8 Hz, 1H), 6.87 (dd, J=7.6, 6.4 Hz, 1H), 3.74-3.70 (m, 2H), 3.51-3.43 (m, 1H), 3.28-3.21 (m, 2H), 2.41-2.34 (m, 2H), 2.16-2.03 (m, 2H).

### Step 5: 4-((1-((4-(2-(2-Aminopyridin-3-yl)-3H-imidazo[4,5-b]pyridin-3-yl)phenyl)methyl-d₂)piperidin-4-yl)(methyl-d3)amino)pyrimidine-2-carbonitrile

To a solution of 3-(3-(4-((4-((methyl-d3)amino)piperidin-1-yl)methyl-d₂)phenyl)-3H-imidazo[4,5-b]pyridin-2-yl)pyridin-2-amine (150 mg, 358.38 μmol), 4-chloropyrimidine-2-carbonitrile (50.0 mg, 358 μmol) in NMP (3 mL) was added DIEA (139 mg, 1.08 mmol). The mixture was stirred at 130° C. for 0.5 hr. After purified by prep-HPLC (column: Welch Xtimate C₁₈ 150×25 mm×5 μm; mobile phase: [water (HCl)-ACN]; gradient: 0%-25% B over 8 min), the crude product was neutralized with saturated NaHCO₃ and extracted with CH₂Cl₂ (10 mL×3). The combined organic layers were washed with brine (10 mL×2), dried over Na₂SO₄, filtered, and concentrated under reduced pressure. 4-((1-((4-(2-(2-Aminopyridin-3-yl)-3H-imidazo[4,5-b]pyridin-3-yl)phenyl)methyl-d₂)piperidin-4-yl)(methyl-d₃)amino)pyrimidine-2-carbonitrile (Example 502, 37.3 mg, yield: 18.2%) was obtained as a yellow solid. MS: m/z=522.3 [M+H]⁺. ¹H NMR (400 MHz, Methanol-d₄) δ 8.34 (dd, J=4.8, 1.2 Hz, 1H), 8.20 (dd, J=8.0, 1.2 Hz, 1H), 8.16 (d, J=6.4 Hz, 1H), 7.98 (dd, J=5.2, 2.0 Hz, 1H), 7.55 (d, J=8.4 Hz, 2H), 7.45-7.39 (m, 3H), 7.32 (dd, J=7.6, 2.0 Hz, 1H), 6.90-6.76 (m, 1H), 6.46 (dd, J=7.6, 5.2 Hz, 1H), 3.30-3.27 (m, 1H), 3.06 (br d, J=12.0 Hz, 2H), 2.31-2.23 (m, 2H), 1.99-1.90 (m, 2H), 1.71-1.67 (m, 2H).

### Example 503: 4-((1-(4-(2-(2-Aminopyridin-3-yl)-3H-imidazo[4,5-b]pyridin-3-yl)benzyl)piperidin-4-yl-4-d)amino)pyrimidine-2-carbonitrile

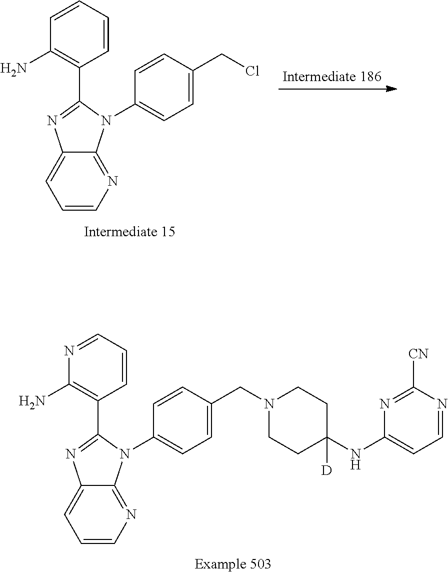 [SMILES: Nc1ccccc1-c1nc2cccnc2n1-c1ccc(CCl)cc1 ; [2H]C1(Nc2ccnc(C#N)n2)CCN(Cc2ccc(-n3c(-c4cccnc4N)nc4cccnc43)cc2)CC1]

To a solution of Intermediate 15 (90 mg, 268 μmol) and Intermediate 186 (60.2 mg, 295 μmol) in DMF (5 mL) were added NaI (8.04 mg, 53.6 μmol) and K₂CO₃ (111 mg, 804 μmol) at 20° C. The reaction mixture was stirred at 20° C. for 12 hr. The mixture was diluted with H₂O (20 mL) was added and extracted with EtOAc (20 mL×3). The combined organic layers were washed with brine (20 mL×2), dried over anhydrous Na₂SO₄, filtered, and concentrated under reduced pressure. After purified by prep-HPLC (column: Phenomenex C18 80×30 mm×5 μm; mobile phase: [water (NH₃H₂O+NH₄HCO₃)-ACN]; gradient: 38%-68% B over 7 min), 4-((I-(4-(2-(2-aminopyridin-3-yl)-3H-imidazo[4,5-b]pyridin-3-yl)benzyl)piperidin-4-yl-4-d)amino)pyrimidine-2-carbonitrile (Example 503, 17.6 mg, 13.0% yield) was obtained as an off-white solid. MS: m/z=504.2 [M+H]⁺. ¹H NMR (400 MHz, Dimethylsulfoxide-d₆) δ 8.33 (d, J=4.8 Hz, 1H), 8.21 (d, J=8.0 Hz, 1H), 8.12-8.03 (m, 2H), 7.99 (dd, J=4.4, 1.2 Hz, 1H), 7.47-7.43 (m, 2H), 7.41-7.36 (m, 3H), 7.16 (dd, J=7.6, 1.6 Hz, 1H), 7.01 (s, 2H), 6.67 (d, J=6.0 Hz, 1H), 6.37 (dd, J=7.6, 4.8 Hz, 1H), 3.56 (s, 2H), 2.85-2.77 (m, 2H), 2.19-2.09 (m, 2H), 1.92-1.82 (m, 2H), 1.53-1.43 (m, 2H).

### Example 504: 4-((1-((4-(2-(2-Aminopyridin-3-yl)-5,6-dimethyl-3H-imidazo[4,5-h]pyridin-3-yl)phenyl)methyl-d₂)piperidin-4-yl)amino)pyrimidine-2-carbonitrile

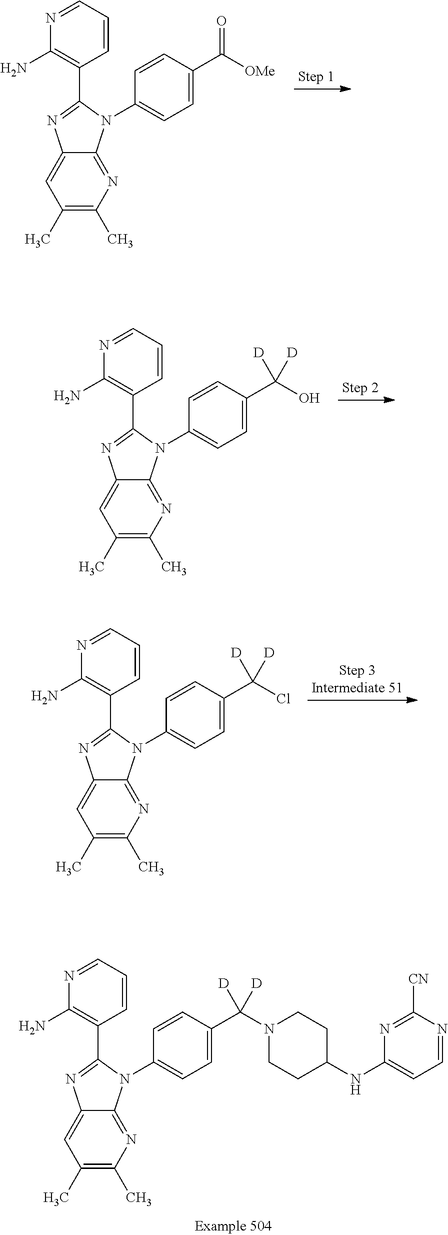 [SMILES: COC(=O)c1ccc(-n2c(-c3cccnc3N)nc3cc(C)c(C)nc32)cc1 ; [2H]C([2H])(O)c1ccc(-n2c(-c3cccnc3N)nc3cc(C)c(C)nc32)cc1 ; [2H]C([2H])(Cl)c1ccc(-n2c(-c3cccnc3N)nc3cc(C)c(C)nc32)cc1 ; [2H]C([2H])(c1ccc(-n2c(-c3cccnc3N)nc3cc(C)c(C)nc32)cc1)N1CCC(Nc2ccnc(C#N)n2)CC1]

### Step 1; (4-(2-(2-Aminopyridin-3-yl)-5,6-dimethyl-3H-imidazo[4,5-b]pyridin-3-yl)phenyl)methan-d₂-ol

To a mixture of methyl 4-(2-(2-aminopyridin-3-yl)-5,6-dimethyl-3H-imidazo[4,5-b]pyridin-3-yl)benzoate (refer to Intermediate 138 for detail procedures, 290 mg, 777 μmol) in THF (20 mL) was added LiAlD₄ (2.5 M in THF, 80 μL). The mixture was degassed and purged with N₂ three times and stirred at 0° C. for 2 hr under N₂ atmosphere. The reaction mixture was quenched with Na₂SO₄·10H₂O (1.0 g) at 0° C., filtered, and concentrated. (4-(2-(2-Aminopyridin-3-yl)-5,6-dimethyl-3H-imidazo[4,5-b]pyridin-3-yl)phenyl)methan-d₂-ol (260 mg, yield: 96%) was obtained as a yellow solid. MS: m/z=347.9 [M+H]⁺.

### Step 2: 3-(3-(4-(Chloromethyl-d₂)phenyl)-5,6-dimethyl-3H-imidazo[4,5-b]pyridin-2-yl)pyridin-2-amine

To a solution of (4-(2-(2-aminopyridin-3-yl)-5,6-dimethyl-3H-imidazo[4,5-b]pyridin-3-yl)phenyl)methan-d₂-ol (260 mg, 748 μmol) in CH₂Cl₂ (15 mL) was added SOCl₂ (534 mg, 4.49 mmol). The mixture was stirred at 40° C. for 1 hr. The mixture was concentrated to give 3-(3-(4-(chloromethyl-d2)phenyl)-5,6-dimethyl-3H-imidazo[4,5-b]pyridin-2-yl)pyridin-2-amine (270 mg, yield: 99%) as a yellow solid. MS: m/z=366.0 [M+H]⁺.

### Step 3: 4-((1-((4-(2-(2-Aminopyridin-3-yl)-5,6-dimethyl-3H-imidazo[4,5-b]pyridin-3-yl)phenyl)methyl-d₂)piperidin-4-yl)amino)pyrimidine-2-carbonitrile

To a solution of 3-(3-(4-(chloromethyl-d2)phenyl)-5,6-dimethyl-3H-imidazo[4,5-b]pyridin-2-yl)pyridin-2-amine (250 mg, 683 μmol) and Intermediate 51 (139 mg, 683 μmol) in DMF (5 mL) were added NaI (51.2 mg, 342 μmol) and K₂CO₃ (472 mg, 342 μmol). The mixture was stirred at 50° C. for 1 hr. The reaction mixture was filtered. After purified by prep-HPLC (column: Waters xbridge 150×25 mm 10 μm; mobile phase: [water (NH₄HCO₃)-ACN]; gradient: 32%-62% B over 10 min), 4-((1-((4-(2-(2-aminopyridin-3-yl)-5,6-dimethyl-3H-imidazo[4,5-b]pyridin-3-yl)phenyl)methyl-d₂)piperidin-4-yl)amino)pyrimidine-2-carbonitrile (Example 504, 66 mg, yield: 18%) was obtained as a yellow solid. MS: m/z=533.2 [M+H]⁺. ¹H NMR (400 MHz, Dimethylsulfoxide-d₆) δ 8.11-8.05 (m, 2H), 7.97-7.94 (m, 2H), 7.45 (d, J=8.0 Hz, 2H), 7.34 (d, J=8.0 Hz, 2H), 7.07-7.03 (m, 3H), 6.67 (d, J=6.0 Hz, 1H), 6.33 (dd, J=7.6, 4.8, 1H), 3.81-3.77 (m, 1H), 2.84-2.80 (m, 2H), 2.45 (s, 3H), 2.39 (s, 3H), 2.16-2.11 (m, 2H), 1.91-1.86 (m, 2H), 1.52-1.45 (m, 2H).

### Example 505: 4-((1-(4-(8-(2-Aminopyridin-3-yl)-9H-purin-9-yl)benzyl)piperidin-4-yl)amino)pyrimidine-2-carbonitrile

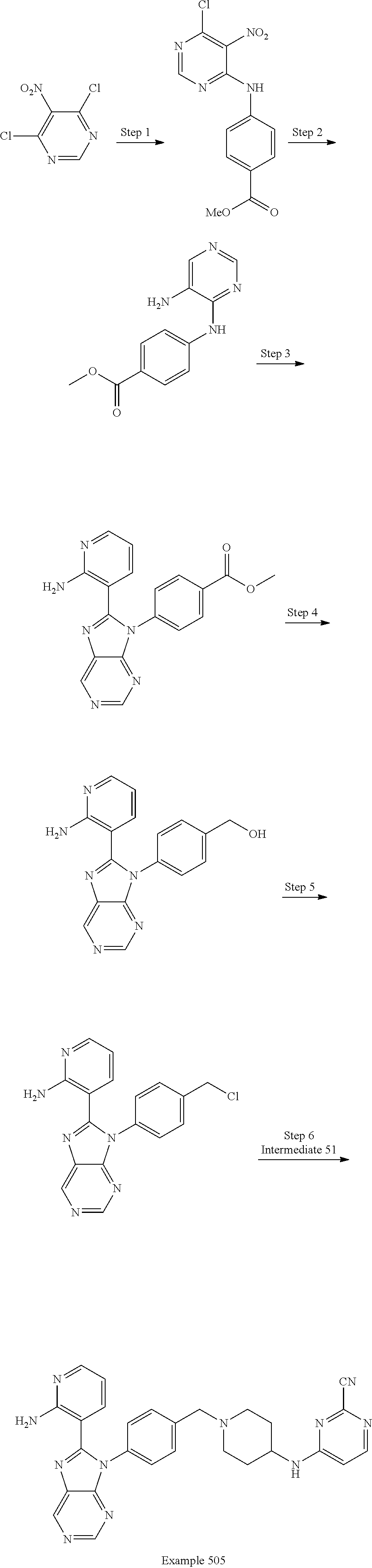

### Step 1: Methyl 4-((6-chloro-5-nitropyrimidin-4-yl)amino)benzoate

To a mixture of 4,6-dichloro-5-nitropyrimidine (3.0 g, 15.5 mmol) and methyl 4-aminobenzoate (1.87 g, 12.4 mmol) in THF (20 mL) was added TEA (1.41 g, 13.9 mmol). The mixture was degassed and purged with N₂ three times and stirred at 0° C. for 4 hr under N₂ atmosphere. The reaction mixture was diluted with water (100 mL) and extracted with EtOAc (50 mL×3). The combined organic layers were washed with brine (100 mL), dried over Na₂SO₄, filtered and concentrated under reduced pressure. After purified by silica gel flash chromatography (Eluent of 0˜8% EtOAc in petroleum ether), methyl 4-((6-chloro-5-nitropyrimidin-4-yl)amino)benzoate (3.0 g, yield: 61%) was obtained as a yellow solid. MS: m/z=308.9 [M+H], ¹H NMR (400 MHz, Dimethylsulfoxide-d₆) δ 10.32 (s, 1H), 8.63 (s, 1H), 7.97 (d, J=8.8 Hz, 2H), 7.74 (d, J=8.8 Hz, 2H), 3.85 (s, 3H)

### Step 2: Methyl 4-((5-aminopyrimidin-4-yl)amino)benzoate

To a solution of methyl 4-((6-chloro-5-nitropyrimidin-4-yl)amino)benzoate (1.5 g, 4.86 mmol) in MeOH (20 mL) was added Pd/C (517 mg, 486 μmol, 10% purity). The mixture was degassed and purged with H₂ three times and stirred at 25° C. for 16 hr under H₂. The reaction mixture was filtered and concentrated to give methyl 4-((5-aminopyrimidin-4-yl)amino)benzoate (1 g, yield: 78%) as a brown solid. MS: m/z=244.9 [M+H]⁺. ¹H NMR (400 MHz, Dimethylsulfoxide-d₆) δ 10.31 (s, 1H), 8.45 (s, 1H), 7.99 (br.s, 4H), 7.82 (s, 1H), 6.77-5.98 (m, 2H), 3.85 (s, 3H).

### Step 3: Methyl 4-(8-(2-aminopyridin-3-yl)-9H-purin-9-yl)benzoate

A mixture of methyl 4-((5-aminopyrimidin-4-yl)amino)benzoate (1.0 g, 4.09 mmol), 2-aminonicotinaldehyde (550 mg, 4.50 mmol) and Cu(Oac)₂ (149 mg, 819 mmol) in CH₃COOH (100 mL) was degassed and purged with N₂ three times. The mixture was stirred at 60° C. for 16 hr under N₂ atmosphere. The reaction mixture was neutralized with NH₃·H₂O (adjusted pH to 7-8). The reaction mixture was diluted with water (100 mL) and extracted with EtOAc (200 mL×3). The combined organic layers were washed with brine (200 mL), dried over Na₂SO₄, filtered and concentrated under reduced pressure. After purified by silica gel flash chromatography (Eluent of 0˜8% MeOH in CH₂Cl₂), methyl 4-(8-(2-aminopyridin-3-yl)-9H-purin-9-yl)benzoate (300 mg, yield: 17%) was obtained as a yellow solid. MS: m/z=347.0 [M+H]⁺, ¹H NMR (400 MHz, Dimethylsulfoxide-d₆) δ 9.29 (s, 1H), 8.94 (s, 1H), 8.10-8.08 (m, 2H), 8.04 (dd, J=4.8, 2.0 Hz, 1H), 7.63-7.61 (m, 2H), 7.35 (d, J=6.4 Hz, 1H), 6.79 (s, 2H), 6.50 (dd, J=7.6, 5.2 Hz, 1H), 3.89 (s, 3H)

### Step 4: (4-(8-(2-Aminopyridin-3-yl)-9H-purin-9-yl)phenyl)methanol

To a mixture of methyl 4-(8-(2-aminopyridin-3-yl)-9H-purin-9-yl)benzoate (300 mg, 866 μmol) in THF (20 mL) was added LiAlH₄ (2.5 M in THF, 693 μL). The mixture was degassed and purged with N₂ three times and stirred at 0° C. for 2 hr under N₂ atmosphere. The reaction mixture was quenched with Na₂SO₄·10H₂O (1.0 g) at 0° C., filtered, and concentrated. (4-(8-(2-Aminopyridin-3-yl)-9H-purin-9-yl)phenyl)methanol (202 mg, yield: 73%) was obtained as a yellow solid. MS: m/z=319.0 [M+H]⁺.

### Step 5: 3-(9-(4-(Chloromethyl)phenyl)-9H-purin-8-yl)pyridin-2-amine

To a solution of (4-(8-(2-aminopyridin-3-yl)-9H-purin-9-yl)phenyl)methanol (202 mg, 635 mol) in CH₂Cl₂ (20 mL) was added SOCl₂ (453 mg, 3.81 mmol). The mixture was stirred at 40° C. for 1 hr. The mixture was concentrated to give 3-(9-(4-(chloromethyl)phenyl)-9H-purin-8-yl)pyridin-2-amine (103 mg, yield: 48%) as a yellow solid. MS: m/z=337.0 [M+H]⁺.

### Step 6: 4-((1-(4-(8-(2-Aminopyridin-3-yl)-9H-purin-9-yl)benzyl)piperidin-4-yl)amino)pyrimidine-2-carbonitrile

To a solution of 3-(9-(4-(chloromethyl)phenyl)-9H-purin-8-yl)pyridin-2-amine (103 mg, 306 μmol) and Intermediate 51 (62 mg, 306 μmol) in DMF (5 mL) were added NaI (22.3 mg, 153 μmol) and K₂CO₃ (127 mg, 918 μmol). The mixture was stirred at 50° C. for 1 hr. The reaction mixture was filtered. After purified by prep-HPLC (column: Waters xbridge 150×25 mm 10 μm; mobile phase: [water (NH₄HCO₃)-ACN]; gradient: 20%-50% B over 11 min), 4-((1-(4-(8-(2-Aminopyridin-3-yl)-9H-purin-9-yl)benzyl)piperidin-4-yl)amino)pyrimidine-2-carbonitrile (Example 505, 5 mg, yield: 3%) was obtained as a yellow solid. MS: m/z=504.1 [M+H]⁺. ¹H NMR (400 MHz, Dimethylsulfoxide-d₆) δ 9.26 (s, 1H), 8.91 (s, 1H), 8.10-8.01 (m, 3H), 7.43-7.41 (m, 4H), 7.25 (dd, J=8.0, 1.6 Hz, 1H), 6.87 (s, 2H), 6.67 (d, J=6.0 Hz, JH), 6.41 (dd, J=7.6, 4.8 Hz, 1H), 3.84-3.78 (m, 1H), 3.56 (s, 2H), 2.83-2.77 (m, 2H), 2.17-2.09 (m, 2H), 1.91-1.82 (m, 2H), 1.52-1.43 (m, 2H)

### Example 506 and 507: (S)-4-((1-(4-(2-(2-Aminopyridin-3-yl)-5-(tetrahydrofuran-3-yl)-3H-imidazo[4,5-b]pyridin-3-yl)benzyl)piperidin-4-yl)amino)pyrimidine-2-carbonitrile & (R)-4-((1-(4-(2-(2-Aminopyridin-3-yl)-5-(tetrahydrofuran-3-yl)-3H-imidazo[4,5-b]pyridin-3-yl)benzyl)piperidin-4-yl)amino)pyrimidine-2-carbonitrile

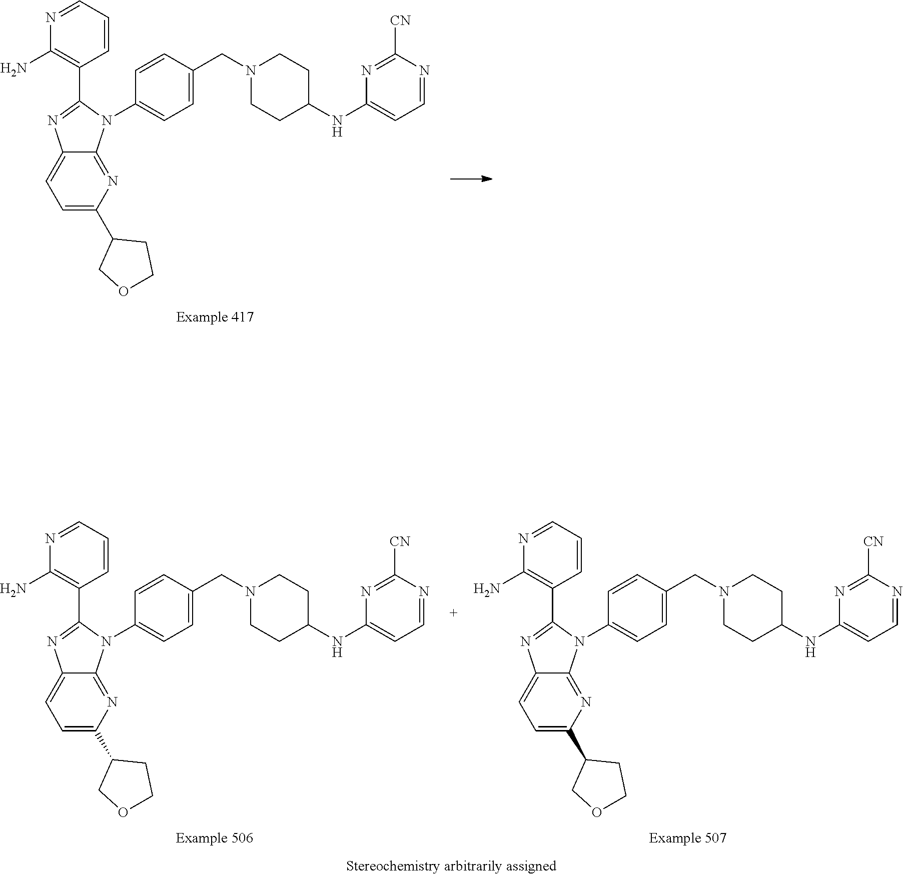 [SMILES: N#Cc1nccc(NC2CCN(Cc3ccc(-n4c(-c5cccnc5N)nc5ccc(C6CCOC6)nc54)cc3)CC2)n1 ; N#Cc1nccc(NC2CCN(Cc3ccc(-n4c(-c5cccnc5N)nc5ccc([C@@H]6CCOC6)nc54)cc3)CC2)n1 ; N#Cc1nccc(NC2CCN(Cc3ccc(-n4c(-c5cccnc5N)nc5ccc([C@H]6CCOC6)nc54)cc3)CC2)n1]

Example 417 (56 mg, 97.8 μmol) was submitted to SFC (column: DAICEL CHIRALPAK IG (250 mm×30 mm, 10 μm); mobile phase: [CO₂-i-PrOH (0.1% NH₃·OH₂O)]; B %: 50%, isocratic elution mode). (S)-4-((1-(4-(2-(2-Aminopyridin-3-yl)-5-(tetrahydrofuran-3-yl)-3H-imidazo[4,5-b]pyridin-3-yl)benzyl)piperidin-4-yl)amino)pyrimidine-2-carbonitrile (Example 506, stereochemistry arbitrarily assigned, 12.9 mg, yield: 23%) was obtained as an off-white solid. MS: m/z=573.3 [M+H]⁺. ¹H NMR (400 MHz, Dimethylsulfoxide-d₆) δ 8.16-8.03 (m, 3H), 7.97 (dd, J=4.8, 1.6 Hz, 1H), 7.46-7.42 (m, 2H), 7.38-7.33 (m, 3H), 7.10 (d, J=7.6 Hz, 1H), 6.96 (s, 2H), 6.67 (d, J=6.0 Hz, 1H), 6.36 (dd, J=7.6, 4.8 Hz, 1H), 4.04 (t, J=8.0 Hz, 1H), 3.93-3.86 (m, 1H), 3.84-3.68 (m, 3H), 3.66-3.56 (m, 3H), 2.86-2.77 (m, 2H), 2.31-2.21 (m, 1H), 2.17-2.05 (m, 3H), 1.94-1.83 (m, 2H), 1.54-1.42 (m, 2H). (R)-4-((1-(4-(2-(2-aminopyridin-3-yl)-5-(tetrahydrofuran-3-yl)-3H-imidazo[4,5-b]pyridin-3-yl)benzyl)piperidin-4-yl)amino)pyrimidine-2-carbonitrile (Example 507, stereochemistry arbitrarily assigned, 15.9 mg, yield: 28%) was obtained as an off-white solid. MS: m/z=573.2 [M+H]⁺. ¹H NMR (400 MHz, Dimethylsulfoxide-d₆) δ 8.16-8.04 (m, 3H), 7.97 (dd, J=4.8, 1.6 Hz, 1H), 7.47-7.42 (m, 2H), 7.39-7.32 (m, 3H), 7.10 (dd, J=7.6, 1.2 Hz, 1H), 6.96 (s, 2H), 6.67 (d, J=6.0 Hz, 1H), 6.36 (dd, J=7.6, 4.8 Hz, 1H), 4.04 (t, 1=8.0 Hz, 1H), 3.92-3.86 (m, 1H), 3.84-3.67 (m, 3H), 3.66-3.56 (m, 3H), 2.85-2.77 (m, 2H), 2.31-2.21 (m, 1H), 2.20-2.03 (m, 3H), 1.93-1.82 (m, 2H), 1.54-1.43 (m, 2H).

### Example 508: 4-((1-(4-(2-(2-Aminopyridin-3-yl)-5-(hydroxymethyl)-3H-imidazo[4,5-b]pyridin-3-yl)benzyl)piperidin-4-yl)amino)pyrimidine-2-carbonitrile

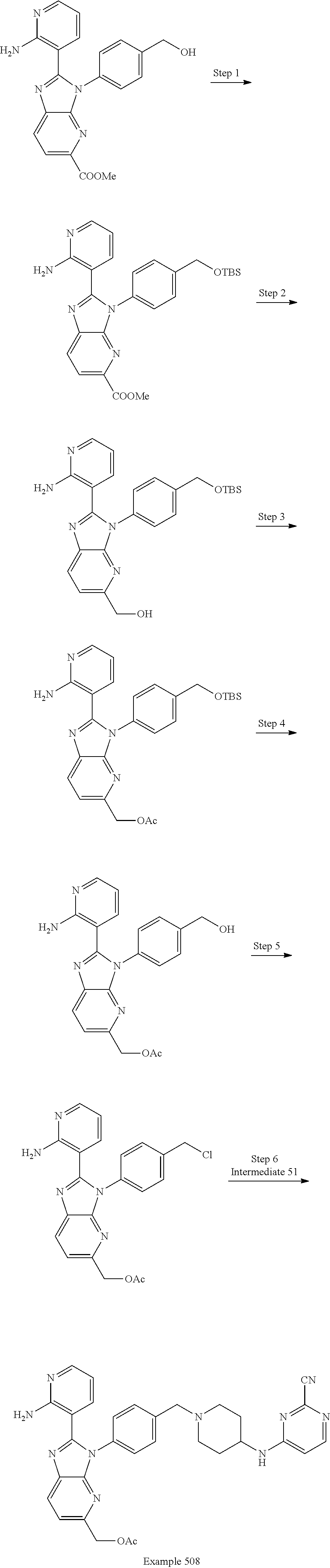

### Step 1: Methyl 2-(2-aminopyridin-3-yl)-3-(4-(((tert-butyldimethylsilyl)oxy)methyl)phenyl)-3H-imidazo[4,5-b]pyridine-5-carboxylate

To a solution of methyl 2-(2-aminopyridin-3-yl)-3-(4-(hydroxymethyl)phenyl)-3H-imidazo[4,5-h]pyridine-5-carboxylate (refer to Intermediate 140 for detail procedures, 900 mg, 2.40 mmol) in CH₂Cl₂ (10 mL) were added imidazole (245 mg, 3.60 mmol) and TBSCl (470 mg, 3.12 mmol) at 0° C. This mixture was stirred at 25° C. for 1 hr. The mixture was quenched with H₂O (30 mL) and extracted with CH₂Cl₂ (30 mL×3). The combined organic layers were washed with brine (50 mL), dried over Na₂SO₄, filtered and concentrated. Methyl 2-(2-aminopyridin-3-yl)-3-(4-(((tert-butyldimethylsilyl)oxy)methyl)phenyl)-3H-imidazo[4,5-b]pyridine-5-carboxylate (1.10 g, yield: 93.7%) was obtained as a yellow solid. MS: mz z=490.4 [M+H]⁺.

### Step 2: (2-(2-Aminopyridin-3-yl)-3-(4-(((tert-butyldimethylsilyl)oxy)methyl)phenyl)-3H-imidazo[4,5-h]pyridin-5-yl)methanol

To a solution of methyl 2-(2-aminopyridin-3-yl)-3-(4-(((tert-butyldimethylsilyl)oxy)methyl)phenyl)-3H-imidazo[4,5-b]pyridine-5-carboxylate (300 mg, 613 μmol) in THF (5 mL) was added LiAlH₄ (34.9 mg, 919 μmol) at 0° C. The mixture was degassed and purged with N₂ three times and stirred at 25° C. for 0.5 hr. The mixture was diluted with THF (20 mL). Na₂SO₄ 10H₂O was added in portions until no bubbles were formed. The resulting mixture was stirred at 25° C. for 20 min and filtered. The filter cake was washed with THF (20 mL×2). The combined filtrate was concentrated to give (2-(2-aminopyridin-3-yl)-3-(4-(((tert-butyldimethylsilyl)oxy)methyl)phenyl)-3H-imidazo[4,5-b]pyridin-5-yl)methanol (244 mg, yield: 86.3%) as a yellow oil. MS: m/z=462.4 [M+H]⁺.

### Step 3: (2-(2-Aminopyridin-3-yl)-3-(4-(((tert-butyldimethylsilyl)oxy)methyl)phenyl)-3H-imidazo[4,5-b]pyridin-5-yl)methyl acetate

To a solution of (2-(2-aminopyridin-3-yl)-3-(4-(((tert-butyldimethylsilyl)oxy)methyl)phenyl)-3H-imidazo[4,5-b]pyridin-5-yl)methanol (244 mg, 529 μmol) in CH₂Cl₂ (10 mL) were added TEA (160 mg, 1.59 mmol), DMAP (6.46 mg, 52.9 μmol) and Ac₂O (80.9 mg, 793 μmol) at 25° C. This mixture was stirred at 25° C. for 0.5 hr. The mixture was quenched with H₂O (50 mL) and extracted with EtOAc (30 mL×3). The combined organic layers were washed with brine (50 mL), dried over Na₂SO₄, filtered and concentrated. (2-(2-Aminopyridin-3-yl)-3-(4-(((tert-butyldimethylsilyl)oxy)methyl)phenyl)-3H-imidazo[4,5-b]pyridin-5-yl)methyl acetate (240 mg, yield: 90.2%) was obtained as a yellow oil. MS: m/z=504.4 [M+H]⁺.

### Step 4: (2-(2-aminopyridin-3-yl)-3-(4-(hydroxymethyl)phenyl)-3H-imidazo[4,5-b]pyridin-5-yl)methyl acetate

To a solution of (2-(2-aminopyridin-3-yl)-3-(4-(((tert-butyldimethylsilyl)oxy)methyl)phenyl)-3H-imidazo[4,5-b]pyridin-5-yl)methyl acetate (240 mg, 476 μmol) in THF (10 mL) was added TBAF (1 M in THF) (1 M, 714.76 μL) at 25° C. This mixture was stirred at 25° C. for 1 hr. The mixture was quenched with H₂O (50 mL) and extracted with EtOAc (30 mL×3). The combined organic layers were washed with brine (50 mL), dried over Na₂SO₄, filtered and concentrated. (2-(2-Aminopyridin-3-yl)-3-(4-(hydroxymethyl)phenyl)-3H-imidazo[4,5-b]pyridin-5-yl)methyl acetate (180 mg, yield: 97.0%) was obtained as a yellow oil. MS: m/z=390.2 [M+H]⁺.

### Step 5: (2-(2-Aminopyridin-3-yl)-3-(4-(chloromethyl)phenyl)-3H-imidazo[4,5-b]pyridin-5-yl)methyl acetate

To a solution of (2-(2-aminopyridin-3-yl)-3-(4-(hydroxymethyl)phenyl)-3H-imidazo[4,5-b]pyridin-5-yl)methyl acetate (180 mg, 462 μmol) in CH₂Cl₂ (10 mL) was added SOCl₂ (82.5 mg, 693 μmol) at 25° C. This mixture was stirred at 25° C. for 1 hr. The reaction mixture was concentrated to give (2-(2-aminopyridin-3-yl)-3-(4-(chloromethyl)phenyl)-3H-imidazo[4,5-b]pyridin-5-yl)methyl acetate (180 mg, yield: 95.5%) as a yellow oil. MS: fftz=408.2 [M+H]⁺.

### Step 6: 4-((1-(4-(2-(2-Aminopyridin-3-yl)-5-(hydroxymethyl)-3H-imidazo[4,5-b]pyridin-3-yl)benzyl)piperidin-4-yl)amino)pyrimidine-2-carbonitrile

To a solution of (2-(2-aminopyridin-3-yl)-3-(4-(chloromethyl)phenyl)-3H-imidazo[4,5-b]pyridin-5-yl)methyl acetate (230 mg, 564 μmol) and Intermediate (126 mg, 620 μmol) in DMF (3 mL) were added K₂CO₃ (117 mg, 846 μmol) and NaI (8.45 mg, 56.4 μmol) at 25° C. This mixture was stirred at 80° C. for 1 hr. The mixture was quenched with H₂O (30 mL) and extracted with EtOAc (30 mL×3). The combined organic layers were washed with brine (50 mL), dried over Na₂SO₄, filtered and concentrated. After purified by prep-HPLC (column: Phenomenex C18 75×30 mm×3 μm; mobile phase: [water (NH₃H₂O+NH₄HCO₃)-ACN]; gradient: 31%-61% B over 7 min), 4-((1-(4-(2-(2-aminopyridin-3-yl)-5-(hydroxymethyl)-3H-imidazo[4,5-b]pyridin-3-yl)benzyl)piperidin-4-yl)amino)pyrimidine-2-carbonitrile (Example 508, 94.5 mg, yield: 31.5%) was obtained as a yellow solid. MS: m/z=533.1 [M+H]⁺. ¹H NMR (400 MHz, Dimethylsulfoxide-d₆) 8.19 (d, J=8.0 Hz, 1H), 8.14-8.02 (m, 2H), 7.97 (dd, J=4.8, 2.0 Hz, 1H), 7.50 (d, J=8.4 Hz, 1H), 7.45 (d, J=8.0 Hz, 2H), 7.36 (d, J=8.4 Hz, 2H), 7.10 (dd, J=7.6, 1.6 Hz, 1H), 7.02 (s, 2H) 6.68 (d, J=6.0 Hz, 1H), 6.35 (dd, J=7.6, 4.8 Hz, 1H), 5.46 (t, J=6.0 Hz, 1H), 4.58 (d, J=6.0 Hz, 2H) 3.87-3.75 (m, 1H) 3.57 (s, 2H) 2.87-2.77 (m, 2H) 2.21-2.09 (m, 2H) 1.95-1.81 (m, 2H) 1.56-1.42 (m, 2H).

### Example 509: 4-((1-(4-(2-(2-Aminopyridin-3-yl)-5-(fluoromethyl)-3H-imidazo[4,5-b]pyridin-3-yl)benzyl)piperidin-4-yl)amino)pyrimidine-2-carbonitrile

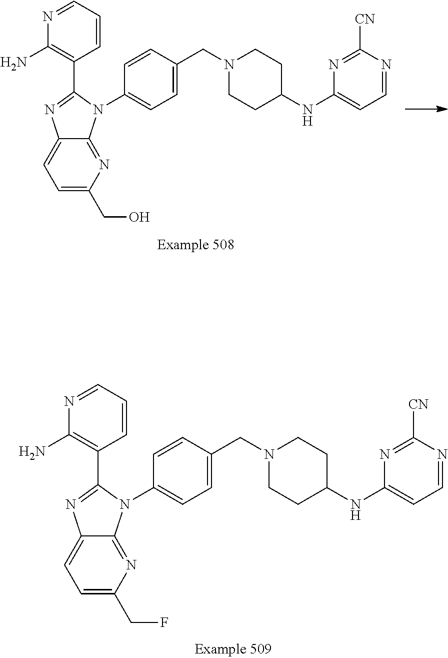 [SMILES: N#Cc1nccc(NC2CCN(Cc3ccc(-n4c(-c5cccnc5N)nc5ccc(CO)nc54)cc3)CC2)n1 ; N#Cc1nccc(NC2CCN(Cc3ccc(-n4c(-c5cccnc5N)nc5ccc(CF)nc54)cc3)CC2)n1]

To a solution of Example 508 (70.0 mg, 131 μmol) in CH₂Cl₂ (3 mL) was added DAST (42.4 mg, 263 μmol) at 0° C. The mixture was stirred at 0° C. for 1 hr. The mixture was quenched with saturated NaHCO₃ (30 mL) and extracted with CH₂Cl₂ (30 mL×3). The combined organic layers were washed with brine (50 mL), dried over Na₂SO₄, filtered and concentrated. After purified by prep-HPLC (column: Phenomenex C18 75×30 mm×3 μm; mobile phase: [water (NH₃H₂O+NH₄HCO₃)-ACN]; gradient: 40%-70% B over 7 min), 4-((1-(4-(2-(2-aminopyridin-3-yl)-5-(fluoromethyl)-3H-imidazo[4,5-b]pyridin-3-yl)benzyl)piperidin-4-yl)amino)pyrimidine-2-carbonitrile (Example 509, 8.20 mg, yield: 11.7%) was obtained as a light yellow solid. MS: m/z=535.1 [M+H]⁺. ¹H NMR (400 MHz, Dimethylsulfoxide-d₆) 8.27 (d, J=8.0 Hz, 1H), 8.18-8.02 (m, 2H), 7.99 (d, J=3.6 Hz, 1H), 7.54 (d, J=8.4 Hz, 1H), 7.46 (d, J=8.0 Hz, 2H), 7.39 (d, J=8.0 Hz, 2H), 7.15 (d, J=7.6 Hz, 1H), 6.99 (s, 2H), 6.68 (d, J=6.0 Hz, 1H), 6.36 (dd, J=8.0, 5.2 Hz, 1H), 5.50 (d, J=47.2 Hz, 2H), 3.89-3.75 (m, 1H), 3.57 (s, 2H), 2.88-2.76 (m, 2H), 2.24-2.07 (m, 2H), 1.96-1.80 (m, 2H), 1.59-1.39 (m, 2H).

### Example 510: 4-((1-(4-(2-(2-Aminopyridin-3-yl)-5-(pyrrolidin-1-yl)-3H-imidazo[4,5-b]pyridin-3-yl)benzyl)piperidin-4-yl)amino)pyrimidine-2-carbonitrile

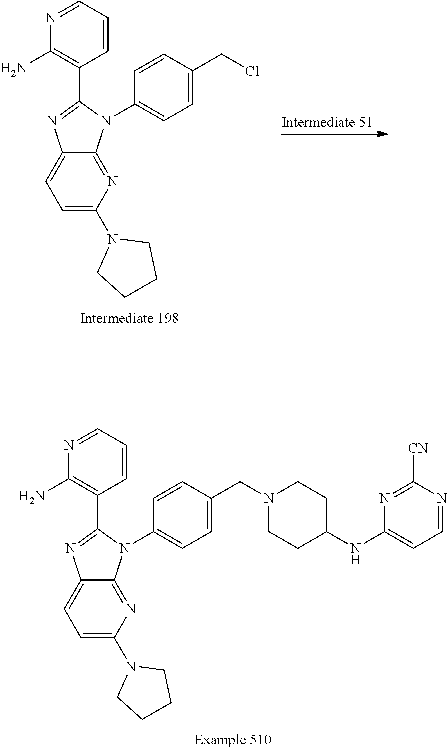 [SMILES: Nc1ncccc1-c1nc2ccc(N3CCCC3)nc2n1-c1ccc(CCl)cc1 ; N#Cc1nccc(NC2CCN(Cc3ccc(-n4c(-c5cccnc5N)nc5ccc(N6CCCC6)nc54)cc3)CC2)n1]

To a solution of Intermediate 198 (105 mg, 259 μmol) and Intermediate 51 (57.9 mg, 285 μmol) in DMF (3 mL) were added NaI (7.76 mg, 51.8 μmol) and K₂CO₃ (107 mg, 776 μmol) at 20° C. The reaction mixture was stirred at 20° C. for 12 hr. The mixture was diluted with H₂O (10 mL) was added at 20° C. and extracted with EtOAc (10 mL×3). The combined organic layers were washed with brine (10 mL×2), dried over anhydrous Na₂SO₄, filtered, and concentrated under reduced pressure. After purified by prep-HPLC (column: Phenomenex C18 75×30 mm×3 μm; mobile phase: [water (NH₃H₂O+NH₄HCO₃)-ACN]; gradient:50%-80% B over 7 min), 4-((1-(4-(2-(2-aminopyridin-3-yl)-5-(pyrrolidin-1-yl)-3H-imidazo[4,5-b]pyridin-3-yl)benzyl)piperidin-4-yl)amino)pyrimidine-2-carbonitrile (Example 510, 65.5 mg, yield: 44%) was obtained as a yellow solid. MS: m/z=572.4 [M+H]⁺. ¹H NMR (400 MHz, Dimethylsulfoxide-d₆) δ 8.11-8.04 (m, 2H), 7.92-7.88 (m, 2H), 7.42 (d, J=8.0 Hz, 2H), 7.31 (d, J=8.4 Hz, 2H), 7.08 (s, 2H), 6.94 (dd, J=7.6, 1.6 Hz, 1H), 6.67 (d, J=6.0 Hz, 1H), 6.50 (d, J=8.8 Hz, 1H), 6.29 (dd, J=8.0, 5.2 Hz, 1H), 3.81 (br.s, 1H), 3.56 (s, 2H), 3.34-3.30 (m, 3H), 2.83-2.78 (m, 2H), 2.17-2.08 (m, 2H), 1.95-1.80 (m, 7H), 1.53-1.42 (m, 2H).

### Example 511: 4-((1-(4-(2-(2-Aminopyridin-3-yl)-5-(2-methoxypyridin-4-yl)-3H-imidazo[4,5-b]pyridin-3-yl)benzyl)piperidin-4-yl)amino)pyrimidine-2-carbonitrile

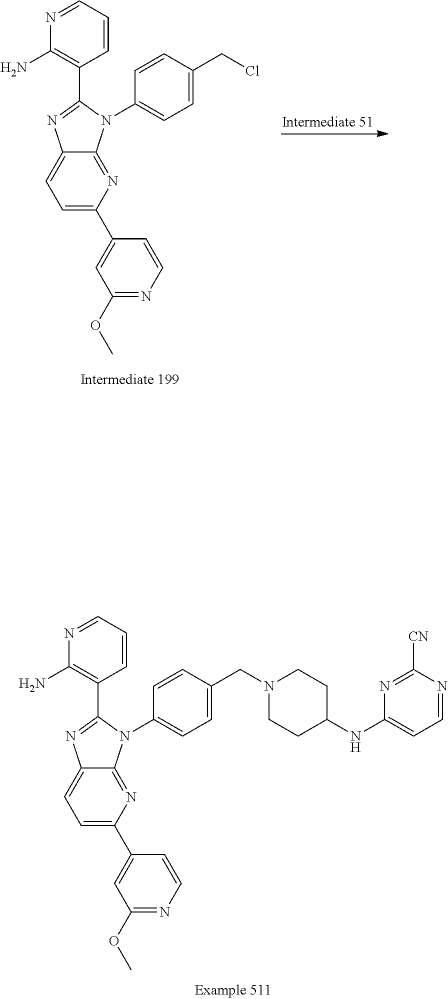 [SMILES: COc1cc(-c2ccc3nc(-c4cccnc4N)n(-c4ccc(CCl)cc4)c3n2)ccn1 ; COc1cc(-c2ccc3nc(-c4cccnc4N)n(-c4ccc(CN5CCC(Nc6ccnc(C#N)n6)CC5)cc4)c3n2)ccn1]

To a solution of Intermediate 199 (540 mg, 1.22 mmol) in DMF (5 mL) were added Intermediate 51 (273 mg, 1.34 mmol) and DIEA (4.88 mmol, 849 μL). The mixture was stirred at 80° C. for 8 hr. After purified by prep-HPLC (column: Phenomenex C18 75×30 mm×3 μm. mobile phase: [water (NH₃H₂O+NH₄HCO₃)-ACN]; gradient: 46%-76% B over 7 min), 4-((1-(4-(2-(2-aminopyridin-3-yl)-5-(2-methoxypyridin-4-yl)-3H-imidazo[4,5-b]pyridin-3-yl)benzyl)piperidin-4-yl)amino)pyrimidine-2-carbonitrile (Example 511, 169 mg, yield: 22.6%) was obtained as a light yellow solid. MS: m/z=610.1 [M+H]⁺. ¹H NMR (400 MHz, Dimethylsulfoxide-d₆) δ 8.31 (d, J=8.4 Hz, 1H), 8.24 (d, J=5.2 Hz, 1H), 8.16-8.04 (m, 3H), 8.01 (dd, J=4.8, 1.6 Hz, 1H), 7.62 (dd, J=5.6, 1.2 Hz, 1H), 7.52-7.47 (m, 2H), 7.46-7.43 (m, 2H), 7.41 (s, 1H), 7.18 (dd, J=8.0, 1.6 Hz, 1H), 7.05 (s, 2H), 6.69 (d, J=5.6 Hz, 1H), 6.39 (dd, J=7.6, 4.8 Hz, 1H), 3.87 (s, 3H), 3.84-3.72 (m, 1H), 3.60 (s, 2H), 3.22-3.11 (m, 2H), 2.83-2.68 (m, 2H), 2.16-2.09 (m, 2H), 1.94-1.85 (m, 2H).

### Example 512: 4-((1-(4-(2-(2-Aminopyrdin-3-yl)-5-(5-methoxypyridin-3-yl)-3H-imidazo[4,5-b]pyridin-3-yl)benzyl)piperidin-4-yl)amino)pyrimidine-2-carbonitrile

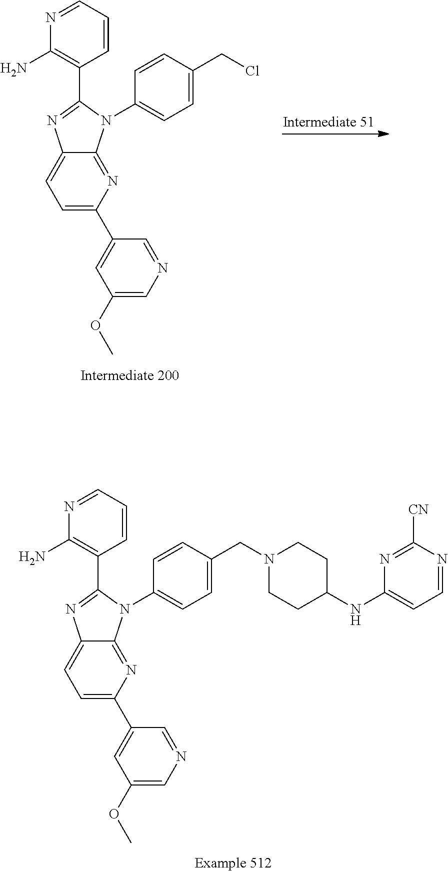 [SMILES: COc1cncc(-c2ccc3nc(-c4cccnc4N)n(-c4ccc(CCl)cc4)c3n2)c1 ; COc1cncc(-c2ccc3nc(-c4cccnc4N)n(-c4ccc(CN5CCC(Nc6ccnc(C#N)n6)CC5)cc4)c3n2)c1]

To a solution of Intermediate 200 (226 mg, 664 μmol) in DMF (3 mL) were added Intermediate 51 (135 mg, 664 μmol), DIEA (2.04 mmol, 356 μL). The mixture was stirred at 80° C. for 8 hr under N₂. The mixture was filtered and the filtrate was concentrated. After purified by prep-HPLC (column: Phenomenex C18 75×30 mm×3 μm; mobile phase: [water (NH₃H₂O+NH₄HCO₃)-ACN]; gradient: 42%-72% B over 7 min), 4-((1-(4-(2-(2-aminopyridin-3-yl)-5-(5-methoxypyridin-3-yl)-3H-imidazo[4,5-b]pyridin-3-yl)benzyl)piperidin-4-yl)amino)pyrimidine-2-carbonitrile (Example 512, 34.3 mg, yield: 11%) was obtained as a light yellow solid. MS: m/z=610.3 [M+H]⁺. ¹H NMR (400 MHz, Dimethylsulfoxide-d₆) δ 8.86 (d, J=1.6 Hz, 1H), 8.32-8.30 (m, 2H), 8.14-8.03 (m, 3H), 8.01 (dd, J=4.8, 2.0 Hz, 1H), 7.92 (dd, J=2.8, 2.0 Hz, 1H), 7.52-7.44 (m, 4H), 7.19 (dd, J=8.0, 2.0 Hz, 1H), 7.06 (s, 2H), 6.67 (d, J=6.0 Hz, 1H), 6.40 (dd, J=7.6, 4.8 Hz, 1H), 3.88 (s, 3H), 3.86-3.75 (m, 1H), 3.59 (s, 2H), 2.89-2.77 (m, 2H), 2.21-2.09 (m, 2H), 1.57-1.42 (m, 2H).

### Example 513: 4-((1-(4-(2-(2-Aminopyridin-3-yl)-5-ethynyl-3H-imidazo[4,5-b]pyridin-3-yl)benzyl)piperidin-4-yl)amino)pyrimidine-2-carbonitrile

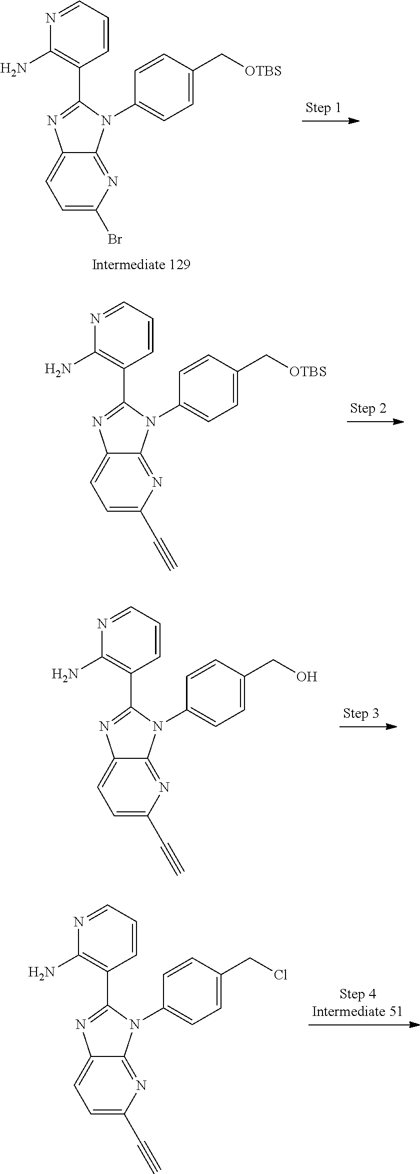 [SMILES: CC(C)(C)[Si](C)(C)OCc1ccc(-n2c(-c3cccnc3N)nc3ccc(Br)nc32)cc1 ; C#Cc1ccc2nc(-c3cccnc3N)n(-c3ccc(CO[Si](C)(C)C(C)(C)C)cc3)c2n1 ; C#Cc1ccc2nc(-c3cccnc3N)n(-c3ccc(CO)cc3)c2n1 ; C#Cc1ccc2nc(-c3cccnc3N)n(-c3ccc(CCl)cc3)c2n1]

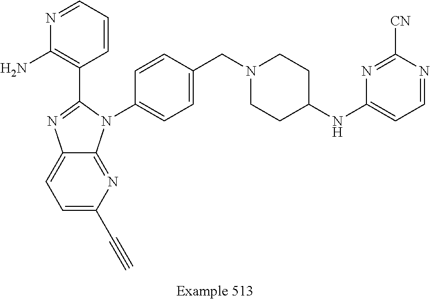 [SMILES: C#Cc1ccc2nc(-c3cccnc3N)n(-c3ccc(CN4CCC(Nc5ccnc(C#N)n5)CC4)cc3)c2n1]

### Step 1: 3-(3-(4-(((Tert-butyldimethylsilyl)oxy)methyl)phenyl)-5-ethynyl-3H-imidazo[4,5-b]pyridin-2-yl)pyridin-2-amine

A solution of Intermediate 129 (1 g, 1.96 mmol), Pd(PPh3)4 (113 mg, 98 μmol) and tributyl(ethynyl)stannane (1.23 g, 3.92 mmol) in 1,4-dioxane (10 mL) was degassed and purged with N₂ three times. The mixture was stirred at 100° C. for 16 hr under N₂ atmosphere. The reaction was concentrated under reduced pressure. After purified by silica gel flash chromatography (Eluent of 30˜50% EtOAc in petroleum ether), 3-(3-(4-(((tert-butyldimethylsilyl)oxy)methyl)phenyl)-5-ethynyl-3H-imidazo[4,5-b]pyridin-2-yl)pyridin-2-amine (900 mg, yield: 60%) was obtained as a black solid. MS: m/z=456.7 [M+H]⁺.

### Step 2: (4-(2-(2-Aminopyridin-3-yl)-5-ethynyl-3H-imidazo[4,5-b]pyridin-3-yl)phenyl)methanol

To a solution of 3-(3-(4-(((tert-butyldimethylsilyl)oxy)methyl)phenyl)-5-ethynyl-3H-imidazo[4,5-b]pyridin-2-yl)pyridin-2-amine (160 mg, 351 μmol) in THF (4 mL) was added TBAF (92 mg, 351 μmol, 1 M). The mixture was stirred at 25° C. for 1 hr. The reaction mixture was quenched with H₂O (10 mL) at 25° C., and extracted with EtOAc (20 mL×3). The combined organic layers were washed with brine (50 mL), dried over Na₂SO₄, filtered and concentrated under reduced pressure. (4-(2-(2-Aminopyridin-3-yl)-5-ethynyl-3H-imidazo[4,5-b]pyridin-3-yl)phenyl)methanol (130 mg, yield: 63%) was obtained as a brown solid. MS: m/z=341.9 [M+H]⁺.

### Step 3: 3-(3-(4-(Chloromethyl)phenyl)-5-ethynyl-3H-imidazo[4,5-b]pyridin-2-yl)pyridin-2-amine

To a solution of (4-(2-(2-aminopyridin-3-yl)-5-ethynyl-3H-imidazo[4,5-b]pyridin-3-yl)phenyl)methanol (130 mg, 381 μmol) in CH₂Cl₂ (5 mL) was added SOCl₂ (90.7 mg, 762 μmol) at 0° C. The mixture was stirred at 40° C. for 0.5 hr. The reaction mixture was quenched with H₂O (1 mL) at 25° C., and concentrated under reduced pressure to give 3-(3-(4-(chloromethyl)phenyl)-5-ethynyl-3H-imidazo[4,5-b]pyridin-2-yl)pyridin-2-amine (130 mg, yield: 52%) as a brown solid. MS: m/z=360.0.

### Step 4: 4-((1-(4-(2-(2-Aminopyridin-3-yl)-5-ethynyl-3H-imidazo[4,5-b]pyridin-3-yl)benzyl)piperidin-4-yl)amino)pyrimidine-2-carbonitrile

To a solution of 3-(3-(4-(chloromethyl)phenyl)-5-ethynyl-3H-imidazo[4,5-b]pyridin-2-yl)pyridin-2-amine (130 mg, 328 μmol) and Intermediate 51 (104 mg, 328 μmol) in DMF (5 mL) were added K₂CO₃ (226.7 mg, 1.64 mmol) and NaI (24.6 mg, 164 μmol). The mixture was stirred at 25° C. for 16 hr. The mixture was filtered, and concentrated. After purified by silica gel flash chromatography (Eluent of 0%˜6% MeOH in CH₂Cl₂) and purified by prep-TLC (CH₂Cl₂:MeOH=10:1), 4-((1-(4-(2-(2-aminopyridin-3-yl)-5-ethynyl-3H-imidazo[4,5-b]pyridin-3-yl)benzyl)piperidin-4-yl)amino)pyrimidine-2-carbonitrile (Example 513, 42.5 mg, yield: 24%) was obtained as a yellow solid. MS: m/z=527.2 [M+H]⁺. ¹H NMR (400 MHz, Methanol-d₄) δ 8.18-8.09 (m, 1H), 8.08-7.94 (m, 2H), 7.61-7.51 (m, 3H), 7.39 (d, J=8.4 Hz, 2H), 7.30 (dd, J=7.6, 1.6 Hz, 1H), 6.60 (d, J=6.0 Hz, 1H), 6.45 (dd, J=7.6, 5.2 Hz, 1H), 4.12-3.82 (m, 1H), 3.75-3.59 (m, 3H), 3.01-2.91 (m, 2H), 2.28 (t, 1=10.8 Hz, 2H), 2.04-1.98 (m, 2H), 1.66-1.59 (m, 2H).

### Example 514: N-(3-(2-(2-Aminopyridin-3-yl)-3-(4-((4-((2-cyanopyrimidin-4-yl)amino)piperidin-1-yl)methyl)phenyl)-3H-imidazo[4,5-b]pyridin-5-yl)phenyl)-N-methylacetamide

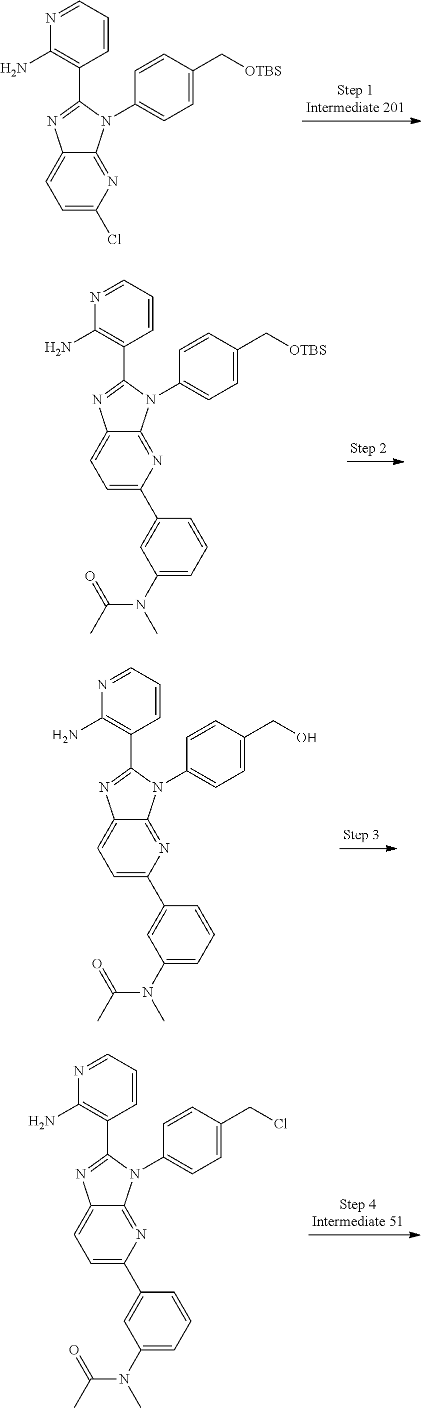

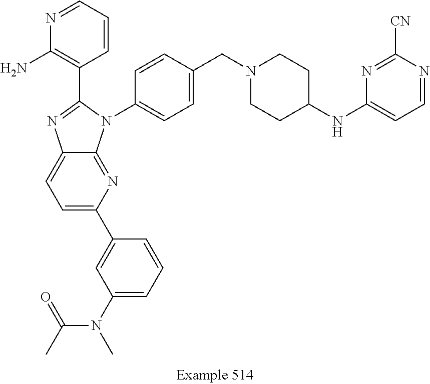 [SMILES: CC(=O)N(C)c1cccc(-c2ccc3nc(-c4cccnc4N)n(-c4ccc(CN5CCC(Nc6ccnc(C#N)n6)CC5)cc4)c3n2)c1]

### Step 1: N-(3-(2-(2-Aminopyridin-3-yl)-3-(4-(((tert-butyldimethylsilyl)oxy)methyl)phenyl)-3H-imidazo[4,5-b]pyridin-5-yl)phenyl)-N-methylacetamide

To a solution of 3-(3-(4-(((tert-butyldimethylsilyl)oxy)methyl)phenyl)-5-chloro-3H-imidazo[4,5-b]pyridin-2-yl)pyridin-2-amine (refer to Intermediate 117 for detail procedures, 500 mg, 1.07 mmol) and Intermediate 201 (325 mg, 1.18 mmol) in 1,4-dioxane (25 mL) and H₂O (5 mL) were added Cs₂CO₃ (1.05 g, 3.22 mmol) and Pd(dppf)Cl₂ (78.5 mg, 107 μmol) at 25° C. The mixture was degassed and purged with N₂ three times and stirred at 100° C. for 2 hr. The mixture was quenched with H₂O (50 mL) and extracted with EtOAc (30 mL×3). The combined organic layers were washed with brine (50 mL), dried over Na₂SO₄, filtered and concentrated. After purified by silica gel flash chromatography (EtoAc in petroleum ether=0-80%4), N-(3-(2-(2-aminopyridin-3-yl)-3-(4-(((tert-butyldimethylsilyl)oxy)methyl)phenyl)-3H-imidazo[4,5-b]pyridin-5-yl)phenyl)-N-methylacetamide (300 mg, yield: 48.3%) was obtained as a yellow solid. MS: m/z=579.4 [M+H]Y.

### Step 2: N-(3-(2-(2-Aminopyridin-3-yl)-3-(4-(hydroxymethyl)phenyl)-3H-imidazo[4,5-b]pyridin-5-yl)phenyl)-N-methylacetamide

To a solution of N-(3-(2-(2-aminopyridin-3-yl)-3-(4-(((tert-butyldimethylsilyl)oxy)methyl)phenyl)-3H-imidazo[4,5-b]pyridin-5-yl)phenyl)-N-methylacetamide (300 mg, 518 μmol) in THF (5 mL) was added TBAF (i M in THF) (1 M, 778 μLL) at 25° C. This mixture was stirred at 25° C. for 1 hr. The mixture was quenched with H₂O (20 mL) and extracted with EtOAc (20 mL×3). The combined organic layers were washed with brine (20 mL), dried over Na₂SO₄, filtered and concentrated. N-(3-(2-(2-Aminopyridin-3-yl)-3-(4-(hydroxymethyl)phenyl)-3H-imidazo[4,5-b]pyridin-5-yl)phenyl)-N-methylacetamide (150 mg, yield: 62.3%) was obtained as yellow oil, which was used directly without purification. MS: m/z=465.3 [M+H]⁺.

### Step 3: N-(3-(2-(2-Aminopyridin-3-yl)-3-(4-(chloromethyl)phenyl)-3H-imidazo[4,5-b]pyridin-5-yl)phenyl)-N-methylacetamide

To a solution of N-(3-(2-(2-aminopyridin-3-yl)-3-(4-(hydroxymethyl)phenyl)-3H-imidazo[4,5-b]pyridin-5-yl)phenyl)-N-methylacetamide (150 mg, 323 μmol) in CH₂Cl₂ (3 mL) was added SOCl₂ (57.6 mg, 484 μmol) at 25° C. This mixture was stirred at 25° C. for 2 hr. The reaction mixture was concentrated to give N-(3-(2-(2-aminopyridin-3-yl)-3-(4-(chloromethyl)phenyl)-3H-imidazo[4,5-b]pyridin-5-yl)phenyl)-N-methylacetamide (120 mg, yield: 76.9%) as a yellow solid, which was used directly without purification. MS: m/z=483.2 [M+H]⁺.

### Step 4: N-(3-(2-(2-Aminopyridin-3-yl)-3-(4-((4-((2-cyanopyrimidin-4-yl)amino)piperidin-1-yl)methyl)phenyl)-3H-imidazo[4,5-b]pyridin-5-yl)phenyl)-N-methylacetamide

To a solution of N-(3-(2-(2-aminopyridin-3-yl)-3-(4-(chloromethyl)phenyl)-3H-imidazo[4,5-b]pyridin-5-yl)phenyl)-N-methylacetamide (120 mg, 248.5 μmol) and Intermediate 51 (55.6 mg, 27 μmol) in DMF (3 mL) were added NaI (3.72 mg, 24.9 μmol) and K₂CO₃ (103 mg, 745 μmol) at 25° C. The mixture was degassed and purged with N₂ three times and stirred at 80° C. for 2 hr. The mixture was quenched with H₂O (30 mL) and extracted with EtOAc (30 mL×3). The combined organic layers were washed with brine (50 mL), dried over Na₂SO₄, filtered and concentrated. After purified by prep-HPLC (column: Phenomenex C18 75×30 mm×3 μm; mobile phase: [water (NH₃H₂O+NH₄HCO₃)-ACN]; gradient: 43%-73% B over 7 min), N-(3-(2-(2-aminopyridin-3-yl)-3-(4-((4-((2-cyanopyrimidin-4-yl)amino)piperidin-1-yl)methyl)phenyl)-3H-imidazo[4,5-b]pyridin-5-yl)phenyl)-N-methylacetamide (Example 514, 41.8 mg, yield: 25.9%) was obtained as a light yellow solid. MS: m/z=650.1 [M+H]⁺. ¹H NMR (400 MHz, Dimethylsulfoxide-d₆) δ 8.28 (d, J=8.4 Hz, 1H), 8.15-7.90 (m, 6H), 7.58-7.42 (m, 5H), 7.36 (d, J=7.2 Hz, 1H), 7.16 (dd, J=7.6, 1.6 Hz, 1H), 7.07 (s, 2H), 6.68 (d, J=6.0 Hz, 1H), 6.38 (dd, J=7.6, 4.8 Hz, 1H), 3.88-3.74 (m, 1H), 3.59 (s, 2H), 3.19 (s, 3H), 2.89-2.75 (m, 2H), 2.21-2.07 (m, 2H), 1.96-1.71 (m, 5H), 1.57-1.41 (m, 2H).

### Example 515: 4-((1-(4-(2-(2-Aminopyridin-3-yl)-5-(3-methoxypyridin-4-yl)-3H-imidazo[4,5-b]pyridin-3-yl)benzyl)piperidin-4-yl)amino)pyrimidine-2-carbonitrile

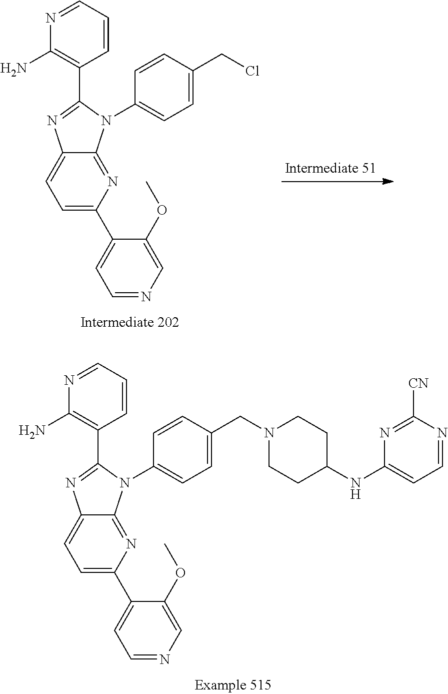 [SMILES: COc1cnccc1-c1ccc2nc(-c3cccnc3N)n(-c3ccc(CCl)cc3)c2n1 ; COc1cnccc1-c1ccc2nc(-c3cccnc3N)n(-c3ccc(CN4CCC(Nc5ccnc(C#N)n5)CC4)cc3)c2n1]

To a solution of Intermediate 202 (150 mg, 339 μmol) in DMF (3 mL) were added DIEA (175 mg, 1.35 mmol) and Intermediate 51 (89.5 mg, 440 μmol). The mixture was stirred at 80° C. for 2 hr. Water (50 mL) was added. The mixture was extracted with EtOAc (20 mL×2). The combined organic layers were dried over anhydrous Na₂SO₄, filtered and concentrated. After purified by prep-HPLC (column: Phenomenex C18 75×30 mm×3 μm; mobile phase: [water (NH₃H₂O+NH₄HCO₃)-ACN]; gradient: 40%-70% B over 7 min), 4-((1-(4-(2-(2-aminopyridin-3-yl)-5-(3-methoxypyridin-4-yl)-3H-imidazo[4,5-b]pyridin-3-yl)benzyl)piperidin-4-yl)amino)pyrimidine-2-carbonitrile (Example 515, 73.5 mg, yield: 36%) was obtained as a light yellow solid. MS: m/z=610.1 [M+H]⁺. ¹H NMR (400 MHz, Dimethylsulfoxide-d₆) δ 8.53 (s, 1H), 8.32-8.25 (m, 2H), 8.14-7.95 (m, 4H), 7.64 (d, J=4.8 Hz, 1H), 7.51-7.41 (m, 4H), 7.11 (d, J=7.6 Hz, 1H), 7.02 (s, 2H), 6.67 (d, J=6.4 Hz, 1H), 6.38 (dd, J=7.6, 4.8 Hz, 1H), 3.98 (s, 3H), 3.87-3.71 (m, 1H), 3.57 (s, 2H), 2.87-2.76 (m, 2H), 2.20-2.09 (m, 2H), 1.94-1.81 (m, 2H), 1.55-1.41 (m, 2H).

### Example 516: 4-((1-(4-(2-(2-Aminopyridin-3-yl)-5-(6-methoxypyridin-3-yl)-3H-imidazo[4,5-b]pyridin-3-yl)benzyl)piperidin-4-yl)amino)pyrimidine-2-carbonitrile

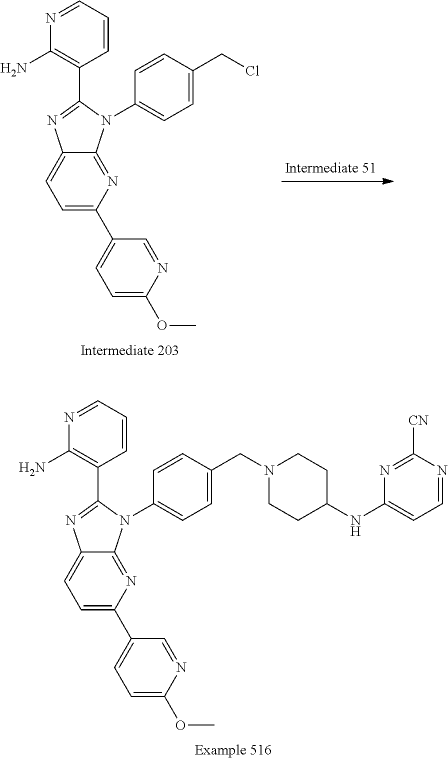 [SMILES: COc1ccc(-c2ccc3nc(-c4cccnc4N)n(-c4ccc(CCl)cc4)c3n2)cn1 ; COc1ccc(-c2ccc3nc(-c4cccnc4N)n(-c4ccc(CN5CCC(Nc6ccnc(C#N)n6)CC5)cc4)c3n2)cn1]

To a solution of Intermediate 203 (240 mg, 542 μmol) in DMF (1 mL) were added DIEA (280 mg, 2.17 mmol) and Intermediate 51 (143 mg, 704 μmol). The mixture was stirred at 25° C. for 2 hr. After purified by prep-HPLC (column: Phenomenex C18 80×30 mm×5 μm; mobile phase: [water (NH₃H₂O+NH₄HCO₃)-ACN]; gradient: 50%-80% B over 7 min), 4-((1-(4-(2-(2-aminopyridin-3-yl)-5-(6-methoxypyridin-3-yl)-3H-imidazo[4,5-b]pyridin-3-yl)benzyl)piperidin-4-yl)amino)pyrimidine-2-carbonitrile (Example 516, 126 mg, yield: 38.3%) was obtained as a yellow solid. MS: m/z=610.2 [M+H]⁺. ¹H NMR (400 MHz, Dimethylsulfoxide-d₆) δ 8.85 (d, J=2.4 Hz, 1H), 8.40-8.20 (m, 2H), 8.15-7.90 (m, 4H), 7.60-7.40 (m, 4H), 7.15 (dd, J=7.6, 1.6 Hz, 1H), 7.05 (s, 2H), 6.92 (d, J=9.2 Hz, 1H), 6.67 (d, J=6.0 Hz, 1H), 6.37 (dd, J=7.6, 4.8 Hz 1H), 3.89 (s, 3H), 3.90-3.70 (m, 1H), 3.59 (s, 2H), 2.83-2.80 (m, 2H), 2.30-2.10 (m, 2H), 1.89-1.82 (m, 2H), 1.60-1.40 (m, 2H).

### Example 517: 4-((1-(4-(5-(3-Aminophenyl)-2-(2-aminopyridin-3-yl)-3H-imidazo[4,5-b]pyridin-3-yl)benzyl)piperidin-4-yl)amino)pyrimidine-2-carbonitrile

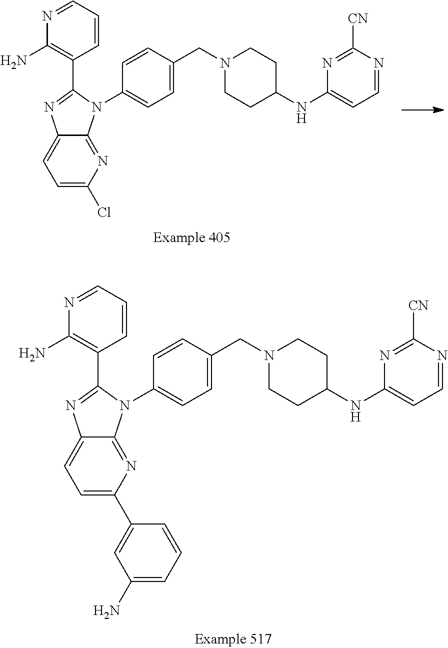 [SMILES: N#Cc1nccc(NC2CCN(Cc3ccc(-n4c(-c5cccnc5N)nc5ccc(Cl)nc54)cc3)CC2)n1 ; N#Cc1nccc(NC2CCN(Cc3ccc(-n4c(-c5cccnc5N)nc5ccc(-c6cccc(N)c6)nc54)cc3)CC2)n1]

To a solution of Intermediate 405 (500 mg, 860 μmol) and (3-aminophenyl)boronic acid (130 mg, 946 μmol) in 1,4-dioxane (5 mL) and H₂O (1 mL) were added Pd(dppf)Cl₂ (62.9 mg, 86.0 μmo) and Cs₂CO₃ (841 mg, 2.58 mmol). The mixture was degassed and purged with N₂ three times and stirred at 90° C. for 16 hr under N₂. The reaction mixture was filtered and concentrated under reduced pressure. The crude product was purified by silica gel flash chromatography (MeOH in CH₂Cl₂=1 to 10%) and prep-HPLC (column: Waters xbridge 150×25 mm 10 μm; mobile phase: [water (NH₄HCO₃)-ACN]; gradient: 35%-55% B over 11 min). 4-((1-(4-(5-(3-Aminophenyl)-2-(2-aminopyridin-3-yl)-3H-imidazo[4,5-b]pyridin-3-yl)benzyl)piperidin-4-yl)amino)pyrimidine-2-carbonitrile (Example 517, 164 mg, yield: 31.1%) was obtained as a light yellow solid. MS: m/z=594.3 [M+H]⁺. ¹H NMR (400 MHz, Methanol-d₄) δ 8.16 (d, J=8.0 Hz, 1H), 8.05-7.92 (m, 2H), 7.87 (d, J=8.4 Hz, 1H), 7.56-7.52 (m, 2H), 7.47-7.43 (m, 2H), 7.41 (t, J=1.6 Hz, 1H), 7.37-7.33 (m, 1H), 7.31 (dd, J=7.6, 1.6 Hz, 1H), 7.17 (t, J=8.0 Hz, 1H), 6.75 (dd, J=7.6, 1.2 Hz, 1H), 6.64-6.55 (m, 1H), 6.47 (dd, J=7.6, 4.8 Hz, 1H), 4.10-3.81 (m, 1H), 3.66 (s, 2H), 3.01-2.93 (m, 2H), 2.32-2.23 (m, 2H), 2.04-1.98 (m, 2H), 1.66-1.58 (m, 2H).

### Example 518 and 519: N-(3-(2-(2-Aminopyridin-3-yl)-3-(4-((4-((2-cyanopyrimidin-4-yl)amino)piperidin-1-yl)methyl)phenyl)-3H-imidazo[4,5-b]pyridin-5-yl)phenyl)isobutyramide & N-(3-(3-(4-((4-((2-cyanopyrimidin-4-yl)amino)piperidin-1-yl)methyl)phenyl)-2-(2-isobutyramidopyridin-3-yl)-3H-imidazo[4,5-b]pyridin-5-yl)phenyl)isobutyramide

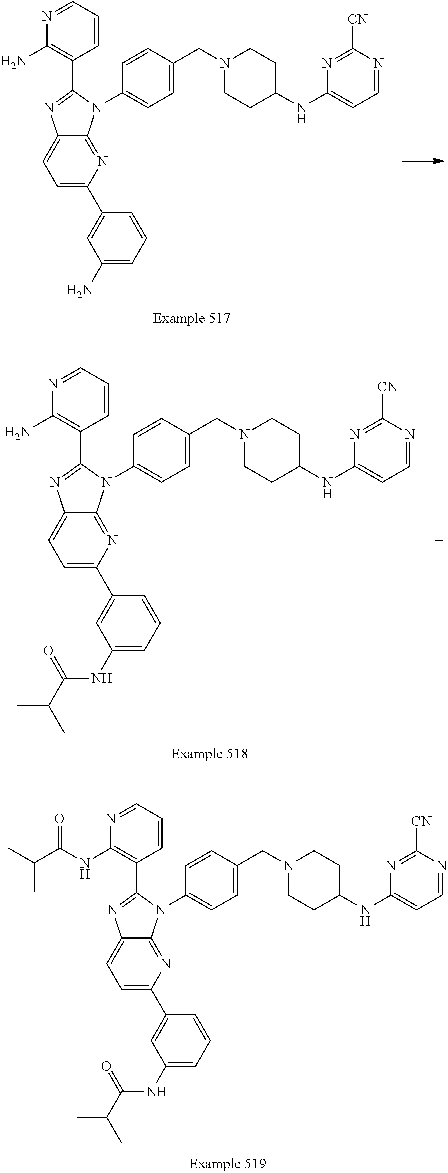 [SMILES: N#Cc1nccc(NC2CCN(Cc3ccc(-n4c(-c5cccnc5N)nc5ccc(-c6cccc(N)c6)nc54)cc3)CC2)n1 ; CC(C)C(=O)Nc1cccc(-c2ccc3nc(-c4cccnc4N)n(-c4ccc(CN5CCC(Nc6ccnc(C#N)n6)CC5)cc4)c3n2)c1 ; CC(C)C(=O)Nc1cccc(-c2ccc3nc(-c4cccnc4NC(=O)C(C)C)n(-c4ccc(CN5CCC(Nc6ccnc(C#N)n6)CC5)cc4)c3n2)c1]

To a solution of Example 517 (220 mg, 371 μmol) and TEA (103 μL, 741 μmol) in THF (5 mL) was added 2-methylpropanoyl chloride (43.4 mg, 408 μmol) dropwise. The mixture was degassed and purged with N₂ three times and stirred at 0° C. for 0.5 hr under N₂. The reaction mixture was filtered and concentrated under reduced pressure. After purified by silica gel flash chromatography (MeOH in CH₂Cl₂=1 to 10%) and prep-HPLC (column: Welch Xtimate C18 150×25 mm×5 μm; mobile phase: [water (HCl)-ACN]; gradient: 12%-42% B over 8 min), N-(3-(2-(2-aminopyridin-3-yl)-3-(4-((4-((2-cyanopyrimidin-4-yl)amino)piperidin-1-yl)methyl)phenyl)-3H-imidazo[4,5-b]pyridin-5-yl)phenyl)isobutyramide (Example 518, 19.8 mg, yield: 7.6%) was obtained as a light yellow solid. MS: m/z=664.3 [M+H]⁺. ¹H NMR (400 MHz, Dimethylsulfoxide-d₆) δ 9.93 (br s, 1H), 8.28 (d, J=8.4 Hz, 1H), 8.21-8.16 (m, 1H), 8.12-8.03 (m, 2H), 8.00 (dd, J=4.8, 1.6 Hz, 1H), 7.87 (d, J=8.4 Hz, 1H), 7.71-7.64 (m, 2H), 7.50-7.43 (m, 4H), 7.37 (t, J=8.0 Hz, 1H), 7.15 (dd, J=7.6, 1.6 Hz, 1H), 6.99 (br s, 2H), 6.68 (d, J=5.6 Hz, 1H), 6.38 (dd, J=7.6, 4.8 Hz, 1H), 3.88-3.73 (m, 1H), 3.59 (s, 2H), 2.88-2.80 (m, 2H), 2.63-2.55 (m, 1H), 2.21-2.12 (m, 2H), 1.93-1.85 (m, 2H), 1.54-1.46 (m, 2H), 1.08 (d, J=6.8 Hz, 6H). N-(3-(3-(4-((4-((2-cyanopyrimidin-4-yl)amino)piperidin-1-yl)methyl)phenyl)-2-(2-isobutyramidopyridin-3-yl)-3H-imidazo[4,5-b]pyridin-5-yl)phenyl)isobutyramide (Example 519, 51 mg, yield: 17.5%) was obtained as a light yellow solid. MS: m/z=734.3 [M+H]⁺. ¹H NMR (400 MHz, Dimethylsulfoxide-d₆) 10.55 (br s, 1H), 9.94 (br s, 1H), 8.44 (dd, J=4.8, 1.6 Hz, 1H), 8.23-8.19 (m, 1H), 8.14 (d, J=8.4 Hz, 1H), 8.09-8.05 (m, 2H), 7.82 (d, J=8.0 Hz, 1H), 7.75-7.68 (m, 4H), 7.46-7.39 (m, 4H), 7.12 (dd, J=7.6, 4.8 Hz, 1H), 6.67 (d, J=6.0 Hz, 1H), 3.87-3.75 (m, 1H), 3.56 (s, 2H), 2.86-2.79 (m, 2H), 2.65-2.58 (m, 2H), 2.18-2.10 (m, 2H), 1.94-1.85 (m, 2H), 1.52-1.45 (m, 2H), 1.10 (d, J=6.8 Hz, 6H), 0.90 (d, J=6.8 Hz, 6H).

### Example 520: Methyl (3-(2-(2-aminopyridin-3-yl)-3-(4-((4-((2-cyanopyrimidin-4-yl)amino)piperidin-1-yl)methyl)phenyl)-3H-imidazo[4,5-b]pyridin-5-yl)phenyl)carbamate

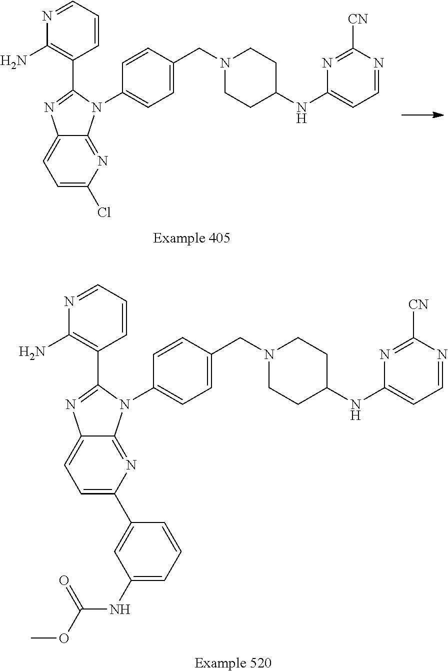 [SMILES: N#Cc1nccc(NC2CCN(Cc3ccc(-n4c(-c5cccnc5N)nc5ccc(Cl)nc54)cc3)CC2)n1 ; COC(=O)Nc1cccc(-c2ccc3nc(-c4cccnc4N)n(-c4ccc(CN5CCC(Nc6ccnc(C#N)n6)CC5)cc4)c3n2)c1]

To a solution of Intermediate 405 (250 mg, 466 μmol) and methyl (3-(4,4,5,5-tetramethyl-1,3,2-dioxaborolan-2-yl)phenyl)carbamate (142 mg, 512 μmol) in 1,4-dioxane (3 mL) and H₂O (0.6 mL) were added Pd(dppf)Cl₂ (68.1 mg, 93.1 μmol) and Cs₂CO₃ (455 mg, 1.40 mmol). The mixture was degassed and purged with N₂ three times and stirred at 100° C. for 16 hr under N₂. The reaction mixture was filtered and concentrated under reduced pressure. The crude product was purified by silica gel flash chromatography (MeOH in CH₂Cl₂=1 to 10%) and then purified by prep-HPLC (column: Welch Xtimate C18 150×25 mm×5 μm; mobile phase: [water (HCl)-ACN]; gradient: 10%-40% B over 8 min). Methyl (3-(2-(2-Aminopyridin-3-yl)-3-(4-((4-((2-cyanopyrimidin-4-yl)amino)piperidin-1-yl)methyl)phenyl)-3H-imidazo[4,5-b]pyridin-5-yl)phenyl)carbamate (Example 520, 80.8 mg, yield: 24.8%) was obtained as a light yellow solid. MS: m/z=652.2 [M+H]⁺. ¹H NMR (400 MHz, Dimethylsulfoxide-d₆) δ 9.74 (br s, 1H), 8.27 (d, J=8.0 Hz, 1H), 8.13-8.02 (m, 3H), 8.00 (dd, J=4.4, 1.6 Hz, 1H), 7.86 (d, J=8.4 Hz, 1H), 7.66-7.61 (m, 1H), 7.51-7.43 (m, 5H), 7.39-7.34 (m, 1H), 7.16 (dd, J=8.0, 2.0 Hz, 1H), 7.00 (br s, 2H), 6.67 (d, J=6.0 Hz, 1H), 6.38 (dd, J=7.6, 4.8 Hz, 1H), 3.87-3.75 (m, 1H), 3.67 (s, 3H), 3.59 (s, 2H), 2.87-2.80 (m, 2H), 2.20-2.11 (m, 2H), 1.93-1.85 (m, 2H), 1.54-1.45 (m, 2H).

### Example 521: 4-((1-(4-(2-(2-Aminopyridin-3-yl)-5-(3-(dimethylamino)phenyl)-3H-imidazo[4,5-b]pyridin-3-yl)benzyl)piperidin-4-yl)amino)pyrimidine-2-carbonitrile

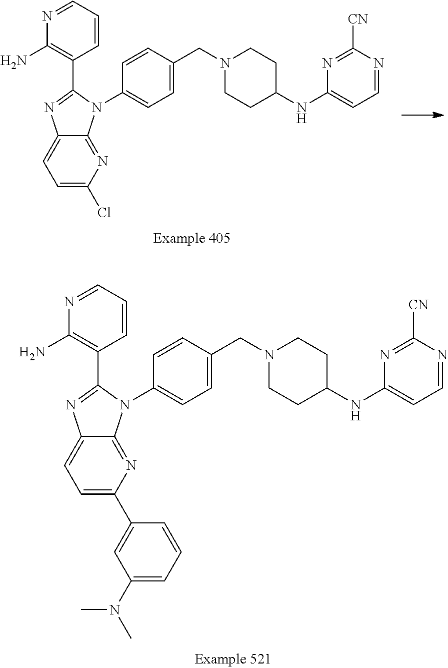 [SMILES: N#Cc1nccc(NC2CCN(Cc3ccc(-n4c(-c5cccnc5N)nc5ccc(Cl)nc54)cc3)CC2)n1 ; CN(C)c1cccc(-c2ccc3nc(-c4cccnc4N)n(-c4ccc(CN5CCC(Nc6ccnc(C#N)n6)CC5)cc4)c3n2)c1]

To a solution of Example 405 (250 mg, 466 μmol) and (3-(dimethylamino)phenyl)boronic acid (84.5 mg, 512 μmol) in 1,4-dioxane (3 mL) and H₂O (0.6 mL) were added Pd(dppf)Cl₂ (68.1 mg, 93.1 μmol) and Cs₂CO₃ (455 mg, 1.40 mmol). The mixture was degassed and purged with N₂ three times and stirred at 100° C. for 16 hr under N₂. The reaction mixture was filtered and concentrated under reduced pressure. The crude product was purified by silica gel flash chromatography (MeOH in CH₂Cl₂=1 to 10%) and then purified by prep-HPLC (column: Waters xbridge 150×25 mm 10 μm; mobile phase: [water (NH₄HCO₃)-ACN]; gradient: 42%-72% B over 11 min). 4-((1-(4-(2-(2-Aminopyridin-3-yl)-5-(3-(dimethylamino)phenyl)-3H-imidazo[4,5-b]pyridin-3-yl)benzyl)piperidin-4-yl)amino)pyrimidine-2-carbonitrile (Example 521, 53.1 mg, yield: 18.1%) was obtained as a light yellow solid. MS: m/z=622.3 [M+H]⁺. ¹H NMR (400 MHz, Dimethylsulfoxide-d₆) 8.23 (d, J=8.4 Hz, 1H), 8.09 (d, J=6.0 Hz, 1H), 8.05 (d, J=7.2 Hz, 1H), 8.00 (dd, J=4.8, 1.6 Hz, 1H), 7.96 (d, J=8.4 Hz, 1H), 7.49-7.44 (m, 4H), 7.42-7.40 (m, 1H), 7.34-7.31 (m, 1H), 7.27-7.23 (m, 1H), 7.19 (dd, J=7.6, 1.6 Hz, 1H), 7.06 (br s, 2H), 6.76 (dd, J=8.0, 2.0 Hz, 1H), 6.68 (d, J=6.0 Hz, 1H), 6.39 (dd, J=7.6, 4.8 Hz, 1H), 3.88-3.74 (m, 1H), 3.59 (s, 2H), 2.92 (s, 6H), 2.85-2.79 (m, 2H), 2.19-2.10 (m, 2H), 1.94-1.86 (m, 2H), 1.53-1.44 (m, 2H).

### Example 522: N-(1-(3-(2-(2-Aminopyridin-3-yl)-3-(4-((4-((2-cyanopyrimidin-4-yl)amino)piperidin-1-yl)methyl)phenyl)-3H-imidazo[4,5-b]pyridin-5-yl)phenyl)piperidin-4-yl)-N-methylacetamide

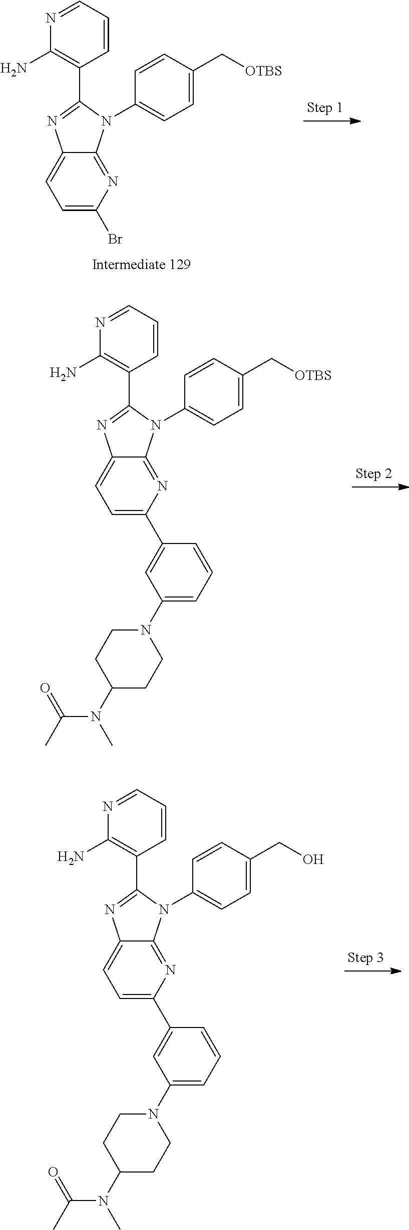 [SMILES: CC(C)(C)[Si](C)(C)OCc1ccc(-n2c(-c3cccnc3N)nc3ccc(Br)nc32)cc1 ; CC(=O)N(C)C1CCN(c2cccc(-c3ccc4nc(-c5cccnc5N)n(-c5ccc(CO[Si](C)(C)C(C)(C)C)cc5)c4n3)c2)CC1 ; CC(=O)N(C)C1CCN(c2cccc(-c3ccc4nc(-c5cccnc5N)n(-c5ccc(CO)cc5)c4n3)c2)CC1]

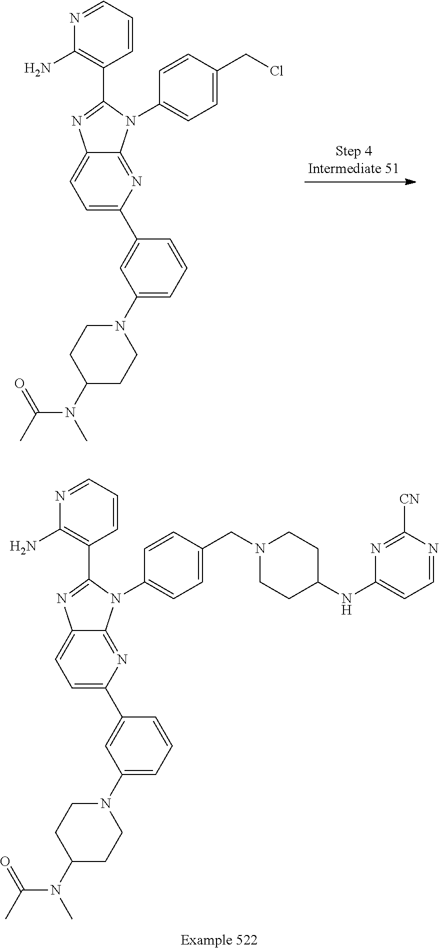

### Step 1: N-(1-(3-(2-(2-Aminopyridin-3-yl)-3-(4-(((tert-butyldimethylsilyl)oxy)methyl)phenyl)-3H-imidazo[4,5-h]pyridin-5-yl)phenyl)piperidin-4-yl)-N-methylacetamide

To a solution of Intermediate 129 (500 mg, 833 μmol) in 1,4-dioxane (25 mL) and H₂O (5 mL) were added Intermediate 204 (328 mg, 916 μmol), Cs₂CO₃ (813.75 mg, 2.50 mmol) and Pd(dppf)Cl₂ (60.9 mg, 83.3 μmol) at 20° C. The reaction mixture was stirred at 100° C. for 2 hr. Water (30 mL) was added. The mixture was extracted with EtOAc (30 mL×3). The combined organic layers were washed with brine (30 mL×2), dried over anhydrous Na₂SO₄, filtered, and concentrated under reduced pressure. After purified by silica gel flash chromatography (MeOH in CH₂Cl₂=0-20%), N-(1-(3-(2-(2-aminopyridin-3-yl)-3-(4-(((tert-butyldimethylsilyl)oxy)methyl)phenyl)-3H-imidazo[4,5-b]pyridin-5-yl)phenyl)piperidin-4-yl)-N-methylacetamide (370 mg, 67.1% yield) was obtained as a purple solid. MS: m/z=662.4 [M+H]⁺.

### Step 2: N-(1-(3-(2-(2-Aminopyridin-3-yl)-3-(4-(hydroxymethyl)phenyl)-3H-imidazo[4,5-b]pyridin-5-yl)phenyl)piperidin-4-yl)-N-methylacetamide

To a solution of -(1-(3-(2-(2-aminopyridin-3-yl)-3-(4-(((tert-butyldimethylsilyl)oxy)methyl)phenyl)-3H-imidazo[4,5-b]pyridin-5-yl)phenyl)piperidin-4-yl)-N-methylacetamide (360 mg, 544 μmol) in THF (12 mL) was added TBAF (1 M in THF, 816 μL). The reaction mixture was stirred at 20° C. for 1 hr. Water (20 mL) was added. The mixture was extracted with EtOAc (20 mL×3). The combined organic layers were washed with brine (20 mL×2), dried over anhydrous Na₂SO₄, filtered, and concentrated under reduced pressure. N-(1-(3-(2-(2-Aminopyridin-3-yl)-3-(4-(hydroxymethyl)phenyl)-3H-imidazo[4,5-b]pyridin-5-yl)phenyl)piperidin-4-yl)-N-methylacetamide (298 mg, 544 μmol, crude) was obtained as a purple solid, which was used directly into the next step. MS: m/z=548.3 [M+H]⁺.

### Step 3: N-(1-(3-(2-(2-Aminopyridin-3-yl)-3-(4-(chloromethyl)phenyl)-3H-imidazo[4,5-b]pyridin-5-yl)phenyl)piperidin-4-yl)-N-methylacetamide

To a solution of N-(1-(3-(2-(2-aminopyridin-3-yl)-3-(4-(hydroxymethyl)phenyl)-3H-imidazo[4,5-b]pyridin-5-yl)phenyl)piperidin-4-yl)-N-methylacetamide (150 mg, 274 μmol) in CH₂Cl₂ (6 mL) was added SOCl₂ (97.8 mg, 822 μmol). The reaction mixture was stirred at 20° C. for 2 hr. The reaction mixture was concentrated to give N-(1-(3-(2-(2-aminopyridin-3-yl)-3-(4-(chloromethyl)phenyl)-3H-imidazo[4,5-b]pyridin-5-yl)phenyl)piperidin-4-yl)-N-methylacetamide (155 mg, 274 μmol, crude) as a gray solid, which was used directly into the next step. MS: m/z=566.1 [M+H]⁺.

### Step 4: N-(1-(3-(2-(2-Aminopyridin-3-yl)-3-(4-((4-((2-cyanopyrimidin-4-yl)amino)piperidin-1-yl)methyl)phenyl)-3H-imidazo[4,5-b]pyridin-5-yl)phenyl)piperidin-4-yl)-N-methylacetamide

To a solution of N-(1-(3-(2-(2-aminopyridin-3-yl)-3-(4-(chloromethyl)phenyl)-3H-imidazo[4,5-b]pyridin-5-yl)phenyl)piperidin-4-yl)-N-methylacetamide (155 mg, 274 μmol) and Intermediate 51 (61.2 mg, 301 μmol) in DMF (5 mL) were added K₂CO₃ (114 mg, 822 μmol) and NaI (8.21 mg, 54.8 μmol) at 20° C. The reaction mixture was stirred at 80° C. for 2 hr. Water (20 mL) was added. The resulting mixture was extracted with EtOAc (20 mL×3). The combined organic layers were washed with brine (20 mL×2), dried over anhydrous Na₂SO₄, filtered, and concentrated under reduced pressure. The residue was re-crystallized from MeCN (5 mL). N-(1-(3-(2-(2-Aminopyridin-3-yl)-3-(4-((4-((2-cyanopyrimidin-4-yl)amino)piperidin-1-yl)methyl)phenyl)-3H-imidazo[4,5-b]pyridin-5-yl)phenyl)piperidin-4-yl)-N-methylacetamide (Example 522, 73.0 mg, yield: 36.4% for 3 steps) was obtained as a gray solid. MS: m/z=733.4 [M+H]⁺. ¹H NMR (400 MHz, Dimethylsulfoxide-d₆) δ 8.24 (d, J=8.4 Hz, 1H), 8.10-7.95 (m, 4H), 7.64 (s, 1H), 7.50-7.42 (m, 5H), 7.28 (t, J=8.0 Hz, 1H), 7.18 (d, J=8.4 Hz, 1H), 7.06 (s, 2H), 6.99 (d, J=8.0 Hz, 1H), 6.67 (s, 1H), 6.39 (s, 1H), 4.43 (s, 1H), 3.87-3.78 (m, 3H), 3.58 (s, 2H), 2.89 (s, 1H), 2.85-2.78 (m, 5H), 2.76-2.73 (m, 1H), 2.65 (s, 1H), 2.15 (s, 2H), 2.06 (s, 1H), 1.98 (s, 2H), 1.88-1.81 (m, 2H), 1.79-1.73 (m, 1H), 1.72-1.64 (m, 1H), 1.57-1.46 (m, 3H).

### Example 523: 4-((1-(4-(2-(2-Aminopyridin-3-yl)-5-(4-fluoro-2-methylphenyl)-3H-imidazo[4,5-b]pyridin-3-yl)benzyl)piperidin-4-yl)amino)pyrimidine-2-carbonitrile

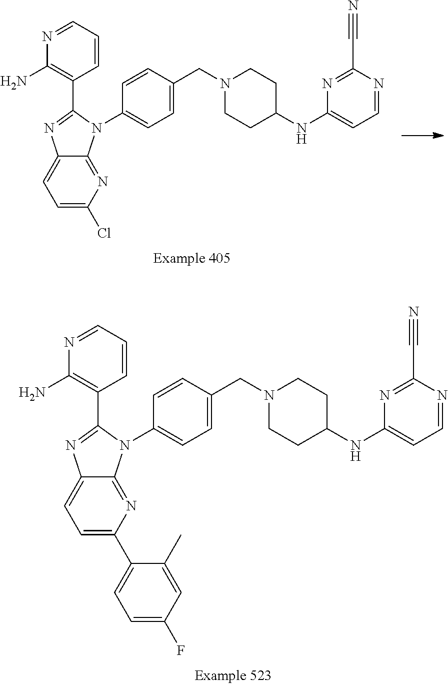 [SMILES: N#Cc1nccc(NC2CCN(Cc3ccc(-n4c(-c5cccnc5N)nc5ccc(Cl)nc54)cc3)CC2)n1 ; Cc1cc(F)ccc1-c1ccc2nc(-c3cccnc3N)n(-c3ccc(CN4CCC(Nc5ccnc(C#N)n5)CC4)cc3)c2n1]

To a solution of Intermediate 405 (180 mg, 335 μmol) in 1,4-dioxane (2 mL) and H₂O (0.4 mL) were added (4-fluoro-2-methylphenyl)boronic acid (67.1 mg, 436 μmol), Cs₂CO₃ (328 mg, 1.01 mmol) and Pd(dppf)Cl₂ (24.5 mg, 33.5 μmol). The mixture was Example stirred at 100° C. for 2 hr under N₂. The reaction mixture was filtered, and the filtrate was concentrated. After purified by prep-HPLC (column: Phenomenex C18 75×30 mm×3 μm; mobile phase: [water (NH₃H₂O+NH₄HCO₃)-ACN]; gradient: 52%-82% B over 7 min), 4-((1-(4-(2-(2-aminopyridin-3-yl)-5-(4-fluoro-2-methylphenyl)-3H-imidazo[4,5-h]pyridin-3-yl)benzyl)piperidin-4-yl)amino)pyrimidine-2-carbonitrile (Example 523, 63.1 mg, yield: 31%) was obtained as a light yellow solid. MS: m/z=611.4 [M+H]⁺. ¹H NMR (400 MHz, Dimethylsulfoxide-d₆) δ 8.27 (d, J=8.4 Hz, 1H), 8.11-8.02 (m, 2H), 8.00 (dd, J=4.8, 2.0 Hz, 1H), 7.52 (d, J=8.4 Hz, 1H), 7.47-7.37 (m, 5H), 7.20-7.07 (m, 3H), 7.02 (s, 2H), 6.66 (d, J=6.4 Hz, 1H), 6.38 (dd, J=7.6, 4.8 Hz, 1H), 3.85-3.74 (m, 1H), 3.55 (s, 2H), 2.79 (d, J=10.4 Hz, 2H), 2.32 (s, 3H), 2.18-2.08 (m, 2H), 1.92-1.79 (m, 2H), 1.53-1.41 (m, 2H). ¹⁹F NMR (400 MHz, Dimethylsulfoxide-d₆) δ −114.725.

### Example 524: 2-(2-(2-aminopyridin-3-yl)-3-(4-((4-((2-cyanopyrimidin-4-yl)amino)piperidin-1-yl)methyl)phenyl)-3H-imidazo[4,5-b]pyridin-5-yl)-5-fluorobenzamide

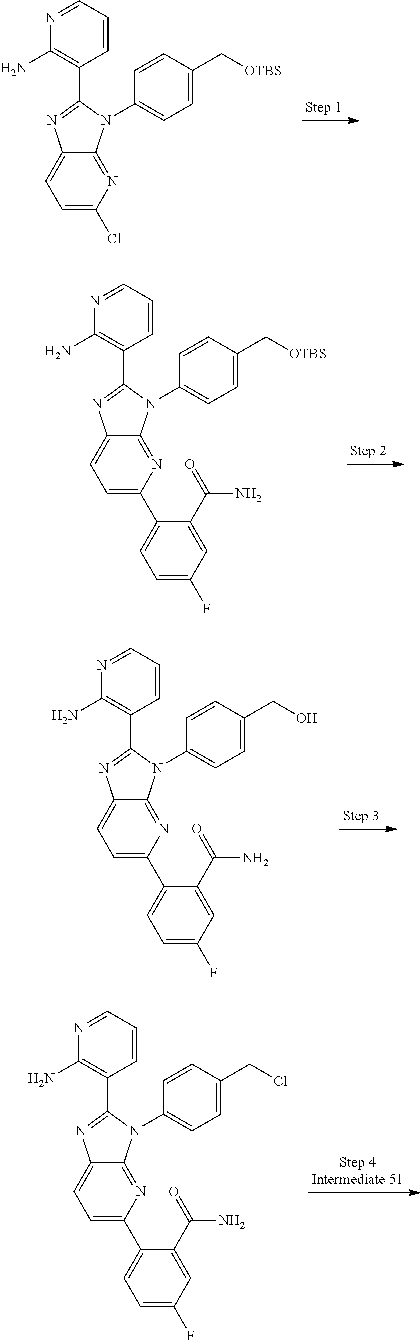 [SMILES: CC(C)(C)[Si](C)(C)OCc1ccc(-n2c(-c3cccnc3N)nc3ccc(Cl)nc32)cc1 ; CC(C)(C)[Si](C)(C)OCc1ccc(-n2c(-c3cccnc3N)nc3ccc(-c4ccc(F)cc4C(N)=O)nc32)cc1 ; NC(=O)c1cc(F)ccc1-c1ccc2nc(-c3cccnc3N)n(-c3ccc(CO)cc3)c2n1 ; NC(=O)c1cc(F)ccc1-c1ccc2nc(-c3cccnc3N)n(-c3ccc(CCl)cc3)c2n1]

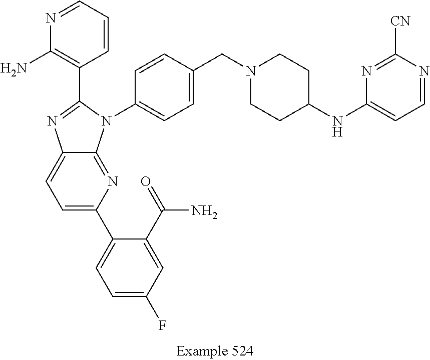 [SMILES: N#Cc1nccc(NC2CCN(Cc3ccc(-n4c(-c5cccnc5N)nc5ccc(-c6ccc(F)cc6C(N)=O)nc54)cc3)CC2)n1]

### Step 1: 2-(2-(2-Aminopyridin-3-yl)-3-(4-(((tert-butyldimethylsilyl)oxy)methyl)phenyl)-3H-imidazo[4,5-b]pyridin-5-yl)-5-fluorobenzamide

To a mixture of 3-(3-(4-(((tert-butyldimethylsilyl)oxy)methyl)phenyl)-5-chloro-3H-imidazo[4,5-b]pyridin-2-yl)pyridin-2-amine (refer to Intermediate 117 for detail procedures, 300 mg, 644 μmol) and (2-cyano-4-fluorophenyl)boronic acid (117 mg, 708 μmol) in 1,4-dioxane (5 mL) and H₂O (1 mL) were added Cs₂CO₃ (629.2 mg, 1.9 mmol, 3 eq) and Pd(dppf)Cl₂ (47.1 mg, 64.4 μmol). The mixture was stirred at 100° C. for 2 hr under N₂. The mixture was diluted with H₂O (50 mL) and extracted with EtOAc (100 mL×2). The combined organic layers were washed with brine (100 mL), dried over anhydrous Na₂SO₄, filtered and concentrated. After purified by silica gel flash chromatography ((ethyl acetate in EtOH=3/1) in petroleum ether=0 to 40%), 2-(2-(2-aminopyridin-3-yl)-3-(4-(((tert-butyldimethylsilyl)oxy)methyl)phenyl)-3H-imidazo[4,5-b]pyridin-5-yl)-5-fluorobenzamide (300 mg, 81.6% yield) was obtained as a brown solid. MS: m/z=569.3 [M+H]⁺. ¹H NMR (400 MHz, Dimethylsulfoxide-d₆) δ 8.23 (d, J=8.4 Hz, 1H), 8.00 (dd, J=4.8, 1.6 Hz, 1H), 7.92-7.84 (m, 1H), 7.65 (dd, J=8.4, 5.6 Hz, 1H), 7.58 (d, J=8.4 Hz, 1H) 7.52-7.47 (m, 2H), 7.47-7.43 (m, 3H), 7.38-7.26 (m, 2H), 7.14 (dd, J=7.6, 1.6 Hz, 1H), 7.02-6.98 (m, 2H), 6.38 (dd, J=8.0, 5.2 Hz, 1H), 4.78 (s, 2H), 0.91 (s, 9H), 0.10 (s, 6H).

### Step 2: 2-(2-(2-Aminopyridin-3-yl)-3-(4-(hydroxymethyl)phenyl)-3H-imidazo[4,5-b]pyridin-5-yl)-5-fluorobenzamide

To a mixture of 2-(2-(2-aminopyridin-3-yl)-3-(4-(((tert-butyldimethylsilyl)oxy)methyl)phenyl)-3H-imidazo[4,5-b]pyridin-5-yl)-5-fluorobenzamide (290 mg, 510 μmol) in THF (1 mL) was added TBAF (1 M in THF, 765 μL), the mixture was stirred at 20° C. for 1 hr. H₂O (50 mL) was added and extracted with EtOAc (30 mL×2). The combined organic layers were washed with brine (50 mL), dried over anhydrous Na₂SO₄, filtered and concentrated, 2-(2-(2-aminopyridin-3-yl)-3-(4-(hydroxymethyl)phenyl)-3H-imidazo[4,5-b]pyridin-5-yl)-5-fluorobenzamide (20 mg, yield: 94.9%) was obtained as a yellow solid. MS: m/z=455.2 [M+H]⁺. ¹H NMR (400 MHz, Dimethylsulfoxide-d₆) δ 8.22 (d, J=8.4 Hz, 1H), 8.00 (dd, J=4.8, 1.8 Hz, 1H), 7.89 (s, 1H), 7.65 (dd, J=8.4, 5.6 Hz, 1H), 7.58 (d, J=8.0 Hz, 1H), 7.49-7.44 (m, 3H), 7.42-7.38 (m, 2H), 7.37-7.31 (m, 1H), 7.30-7.24 (m, 1H), 7.17 (dd, J=7.6, 2.0 Hz, 1H), 7.09-6.88 (m, 1H), 6.42 (dd, J=7.6, 5.2 Hz, 1H), 4.56 (d, J=6.0 Hz, 1H).

### Step 3: 2-(2-(2-Aminopyridin-3-yl)-3-(4-(chloromethyl)phenyl)-3H-imidazo[4,5-b]pyridin-5-yl)-5-fluorobenzamide

To a mixture of 2-(2-(2-aminopyridin-3-yl)-3-(4-(hydroxymethyl)phenyl)-3H-imidazo[4,5-b]pyridin-5-yl)-5-fluorobenzamide (200 mg, 440 μmol) in CH₂Cl₂ (2 mL) was added SOCl₂ (157.1 mg, 1.32 mmol), the mixture was stirred at 20° C. for 1 hr. The mixture was concentrated, 2-(2-(2-aminopyridin-3-yl)-3-(4-(chloromethyl)phenyl)-3H-imidazo[4,5-b]pyridin-5-yl)-5-fluorobenzamide (208 mg, yield: 99.9%) was obtained as a yellow solid. MS: m/z=473.1 [M+H]⁺.

### Step 4: 2-(2-(2-Aminopyridin-3-yl)-3-(4-((4-((2-cyanopyrimidin-4-yl)amino)piperidin-1-yl)methyl)phenyl)-3H-imidazo[4,5-b]pyridin-5-yl)-5-fluorobenzamide

To a mixture of 2-(2-(2-aminopyridin-3-yl)-3-(4-(chloromethyl)phenyl)-3H-imidazo[4,5-b]pyridin-5-yl)-5-fluorobenzamide (200 mg, 423 μmol) and Intermediate 51 (94.6 mg, 465 μmol) in DMF (4 mL) was added DIEA (164 mg, 1.27 mmol). The mixture was stirred at 80° C. for 2 hr. H₂O (50 mL) was added and extracted with EtOAc (50 mL×2). The combined organic layers were washed with brine (50 mL), dried over anhydrous Na₂SO₄, filtered and concentrated. After purified byprep-HPLC (column. C18 150×30 mm; mobile phase: [water (NH₃H₂O+NH₄HCO₃)-ACN]; gradient: 46%-76% B over 7 min), 2-(2-(2-aminopyridin-3-yl)-3-(4-((4-((2-cyanopyrimidin-4-yl)amino)piperidin-1-yl)methyl)phenyl)-3H-imidazo[4,5-b]pyridin-5-yl)-5-fluorobenzamide (Example 524, 100 mg, yield: 37%) was obtained as a yellow solid. MS: m/z=640.2 [M+H]⁺. ¹H NMR (400 MHz, Dimethylsulfoxide-d₆) δ 8.23 (d, J=8.0 Hz, 1H), 8.16-7.96 (m, 3H), 7.89 (s, 1H), 7.67 (dd, J=8.4, 5.6 Hz, 1H), 7.59 (d, J=8.4 Hz, 1H), 7.50-7.39 (m, 5H), 7.38-7.32 (m, 1H), 7.30-7.26 (m, 1H), 7.28 (dd, J=9.1, 2.0 Hz, 1H) 7.11 (dd, J=7.6, 2.0 Hz, 1H), 7.02 (br s, 2H), 6.67 (d, J=6.4 Hz, 1H), 6.38 (dd, J=7.6, 4.8 Hz, 1H), 3.88-3.74 (m, 1H), 3.54 (s, 2H), 2.85-2.75 (m, 2H), 2.21-2.06 (m, 2H), 1.94-1.79 (m, 2H), 1.57-1.41 (m, 2H). ¹⁹F NMR (400 MHz, Dimethylsulfoxide-d₆) δ −114.071.

### Example 525: 4-((1-(4-(2-(2-Aminopyridin-3-yl)-5-(2-cyano-4-fluorophenyl)-3H-imidazo[4,5-b]pyridin-3-yl)benzyl)piperidin-4-yl)amino)pyrimidine-2-carbonitrile

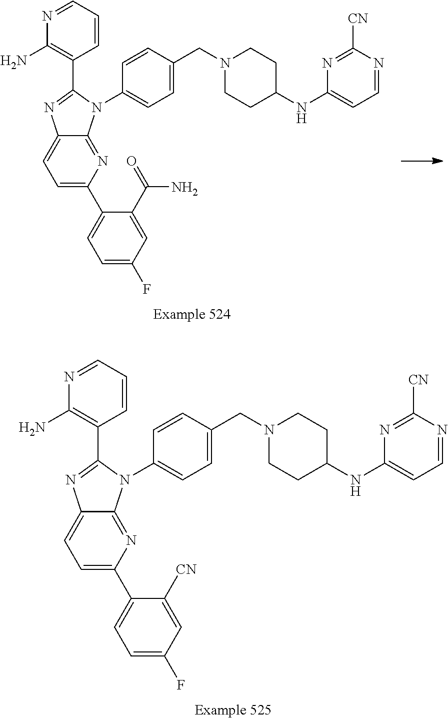 [SMILES: N#Cc1nccc(NC2CCN(Cc3ccc(-n4c(-c5cccnc5N)nc5ccc(-c6ccc(F)cc6C(N)=O)nc54)cc3)CC2)n1 ; N#Cc1nccc(NC2CCN(Cc3ccc(-n4c(-c5cccnc5N)nc5ccc(-c6ccc(F)cc6C#N)nc54)cc3)CC2)n1]

To a mixture of Example 524 (60 mg, 93.8 μmol) in CH₂Cl₂ (1 mL) were added POCl₃ (86.3 mg, 563 μmol) and Py (59.4 mg, 750 μmol). The mixture was stirred at 25° C. for 1 hr. Water (20 mL) was added. The pH was adjusted to 10 with saturated NaHCO₃ (10 mL). The mixture was extracted with CH₂Cl₂ (30 mL×2). The combined organic layers were washed with brine (30 mL), dried over anhydrous Na₂SO₄, filtered and concentrated. After purified by prep-HPLC (column: Xtimate C18 150×40 mm×10 μm; mobile phase: [water (FA)-ACN]; gradient: 15%-45% B over 7 min), 4-((1-(4-(2-(2-aminopyridin-3-yl)-5-(2-cyano-4-fluorophenyl)-3H-imidazo[4,5-b]pyridin-3-yl)benzyl)piperidin-4-yl)amino)pyrimidine-2-carbonitrile (Example 525, 13.7 mg, yield: 23.5%) was obtained as a yellow solid. MS: m/z=622.1 [M+H]⁺. ¹H NMR (400 MHz, Dimethylsulfoxide-d₆) δ 8.38 (d, J=8.4 Hz, 1H), 8.11-8.00 (m, 3H), 7.98-7.89 (m, 2H) 7.83 (d, J=8.4 Hz, 1H), 7.73-7.67 (m, 1H), 7.51-7.39 (m, 4H), 7.19 (dd, J=7.6, 1.6 Hz, 1H), 7.00 (br s, 2H), 6.66 (d, J=6.0 Hz, 1H), 6.40 (dd, J=8.0, 4.8 Hz, 1H), 3.87-3.72 (m, 1H), 3.55 (s, 2H), 2.86-2.73 (m, 2H), 2.19-2.05 (m, 2H), 1.94-1.79 (m, 2H), 1.57-1.40 (m, 2H). ¹⁹F NMR (376.5 MHz, Dimethylsulfoxide-d₆) δ −112.011.

### Example 526: 4-((1-(4-(2-(2-Aminopyridin-3-yl)-5-(4-fluoro-2-(trifluoromethyl)phenyl)-3H-imidazo[4,5-b]pyridin-3-yl)benzyl)piperidin-4-yl)amino)pyrimidine-2-carbonitrile

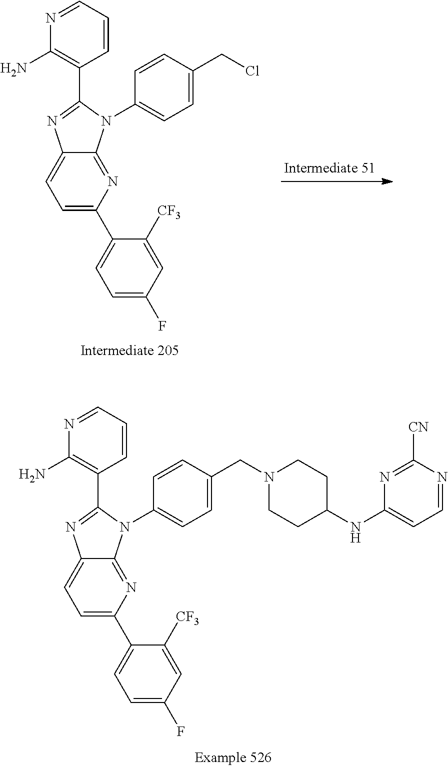 [SMILES: Nc1ncccc1-c1nc2ccc(-c3ccc(F)cc3C(F)(F)F)nc2n1-c1ccc(CCl)cc1 ; N#Cc1nccc(NC2CCN(Cc3ccc(-n4c(-c5cccnc5N)nc5ccc(-c6ccc(F)cc6C(F)(F)F)nc54)cc3)CC2)n1]

To a mixture of Intermediate 205 (100 mg, 200.9 μmol) and Intermediate 51 (44.9 mg, 220.9 μmol) in DMF (2 mL) was added DIEA (77.9 mg, 602.6 μmol), the mixture was stirred at 80° C. for 12 hr. H₂O (50 mL) was added and extracted with EtOAc (50 mL×2). The combined organic layers were washed with brine (50 mL), dried over anhydrous Na₂SO₄, filtered and concentrated. After purified by prep-HPLC (column: C18 150*30 mm, mobile phase: [water (NH₃H₂O+NH₄HCO₃)-ACN]; gradient: 60%-90% B over 7 min), 4-((1-(4-(2-(2-aminopyridin-3-yl)-5-(4-fluoro-2-(trifluoromethyl)phenyl)-3H-imidazo[4,5-b]pyridin-3-yl)benzyl)piperidin-4-yl)amino)pyrimidine-2-carbonitrile (Example 526, 65.8 mg, yield: 49.3%) was obtained as a yellow solid. MS: m/z=665.1 [M+H]⁺. ¹H NMR (400 MHz, Dimethylsulfoxide-d₆) δ 8.30 (d, J=8.0 Hz, 1H), 8.20-7.93 (m, 3H), 7.74 (dd, J=8.2, 2.0 Hz, 1H), 7.66-7.60 (m, 2H), 7.52 (d, J=8.0 Hz, 1H), 7.45-7.33 (m, 4H), 7.17 (dd, J=7.6, 2.0 Hz, 1H), 7.03 (s, 2H), 6.66 (d, J=6.0 Hz, 1H), 6.38 (dd, J=7.6, 4.8 Hz, 1H), 3.84-3.73 (m, 1H), 3.53 (s, 2H), 2.86-2.74 (m, 2H), 2.19-2.08 (m, 2H), 1.92-1.78 (m, 2H), 1.57-1.37 (m, 2H). ¹⁹F NMR (400 MHz, Dimethylsulfoxide-d₆) δ −55.876, −111.904.

### Example 527: 4-((1-(4-(2-(2-Aminopyridin-3-yl)-5-(o-tolyl)-3H-imidazo[4,5-b]pyridin-3-yl)benzyl)piperidin-4-yl)amino)pyrimidine-2-carbonitrile

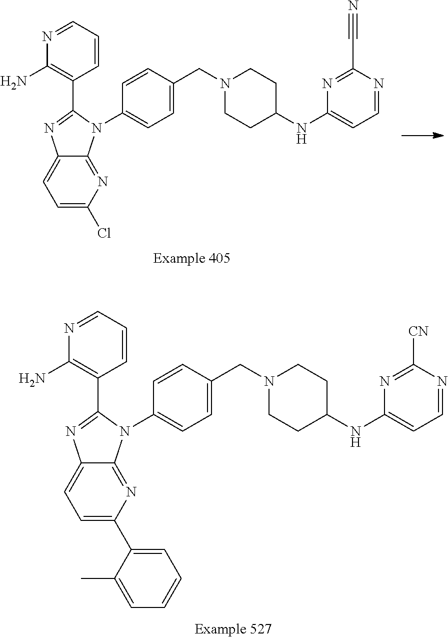 [SMILES: N#Cc1nccc(NC2CCN(Cc3ccc(-n4c(-c5cccnc5N)nc5ccc(Cl)nc54)cc3)CC2)n1 ; Cc1ccccc1-c1ccc2nc(-c3cccnc3N)n(-c3ccc(CN4CCC(Nc5ccnc(C#N)n5)CC4)cc3)c2n1]

To a solution of Example 405 (118 mg, 220 μmol) in 1,4-dioxane (1 mL) and H₂O (0.2 mL) were added o-tolylboronic acid (38.8 mg, 286 μmol), Pd(dppf)Cl₂ (16.1 mg, 22 μmol) and Cs₂CO₃ (215 mg, 659 μmol). The mixture was stirred at 100° C. for 2 hr under N₂. The reaction mixture was filtered and the filtrate was concentrated. After purified by prep-HPLC (column: Phenomenex C18 75×30 mm×3 μm; mobile phase: [water (NH₃H₂O+NH₄HCO₃)-ACN]; gradient: 52%-82% B over 7 min), 4-((1-(4-(2-(2-aminopyridin-3-yl)-5-(o-tolyl)-3H-imidazo[4,5-b]pyridin-3-yl)benzyl)piperidin-4-yl)amino)pyrimidine-2-carbonitrile (Example 527, 26.2 mg, yield: 20%) was obtained as a light-yellow solid. MS: m/z=593.2 [M+H]⁺. ¹H NMR (400 MHz, Dimethylsulfoxide-d₆) δ 8.26 (d, J=8.4 Hz, 1H), 8.12-7.95 (m, 3H), 7.53 (d, J=8.4 Hz, 1H), 7.47-7.37 (m, 5H), 7.31-7.25 (m, 3H), 7.17 (d, J=6.4 Hz, 1H), 7.02 (s, 2H), 6.67 (d, J=6.0 Hz, 1H), 6.38 (dd, J=7.6, 4.8 Hz, 1H), 3.83-3.73 (m, 1H), 3.55 (s, 2H), 2.79 (d, J=10.8 Hz, 2H), 2.31 (s, 3H), 2.18-2.05 (m, 2H), 1.93-1.79 (m, 2H), 1.54-1.39 (m, 2H).

### Example 528: 4-((1-(4-(2-(2-Aminopyridin-3-yl)-5-(m-tolyl)-3H-imidazo[4,5-h]pyridin-3-yl)benzyl)piperidin-4-yl)amino)pyrimidine-2-carbonitrile

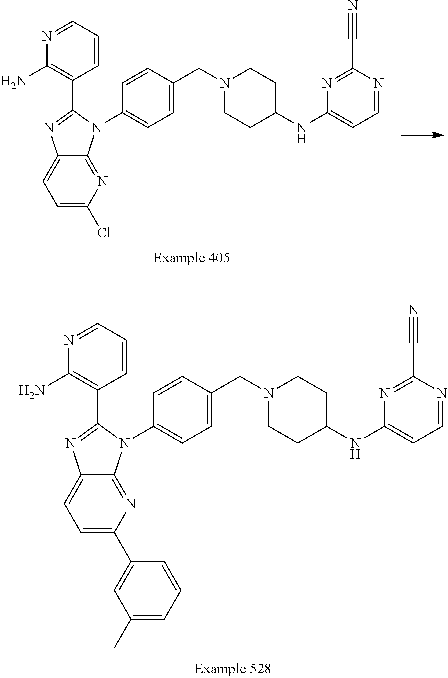 [SMILES: N#Cc1nccc(NC2CCN(Cc3ccc(-n4c(-c5cccnc5N)nc5ccc(Cl)nc54)cc3)CC2)n1 ; Cc1cccc(-c2ccc3nc(-c4cccnc4N)n(-c4ccc(CN5CCC(Nc6ccnc(C#N)n6)CC5)cc4)c3n2)c1]

To a solution of Example 405 (180 mg, 335 μmol) in 1,4-dioxane (2 mL) and H₂O (0.4 mL) were added m-tolylboronic acid (59.2 mg, 436 μmol), Pd(dppf)Cl₂ (24.5 mg, 33.5 μmol) and Cs₂CO₃ (328 mg, 1.01 mmol). The mixture was stirred at 100° C. for 2 hr under N₂. The reaction mixture was filtered, and the filtrate was concentrated. After purified by prep-HPLC (column: Phenomenex C18 75×30 mm×3 μm; mobile phase: [water (NH₃H₂O+NH₄HCO₃)-ACN]; gradient: 54%-84% B over 7 min), 4-((1-(4-(2-(2-aminopyridin-3-yl)-5-(m-tolyl)-3H-imidazo[4,5-b]pyridin-3-yl)benzyl)piperidin-4-yl)amino)pyrimidine-2-carbonitrile (Example 528, 67.8 mg, yield: 34%) was obtained as a light-yellow solid. MS: m/z=593.4 [M+H]⁺. ¹H NMR (400 MHz, Dimethylsulfoxide-d₆) δ 8.25 (dd, J=8.0, 3.2 Hz, 1H), 8.13-7.92 (m, 4H), 7.86-7.78 (m, 2H), 7.53-7.41 (m, 4H), 7.38-7.30 (m, 1H), 7.23-7.12 (m, 2H), 7.03 (s, 2H), 6.68 (s, 1H), 6.42-6.33 (m, 1H), 3.89-3.74 (m, 1H), 3.60 (s, 2H), 2.89-2.77 (m, 2H), 2.36 (s, 3H), 2.21-2.08 (m, 2H), 1.96-1.83 (m, 2H), 1.59-1.43 (m, 2H).

### Example 529: 4-((1-(4-(2-(2-Aminopyridin-3-yl)-5-(p-tolyl)-3H-imidazo[4,5-h]pyridin-3-yl)benzyl)piperidin-4-yl)amino)pyrimidine-2-carbonitrile

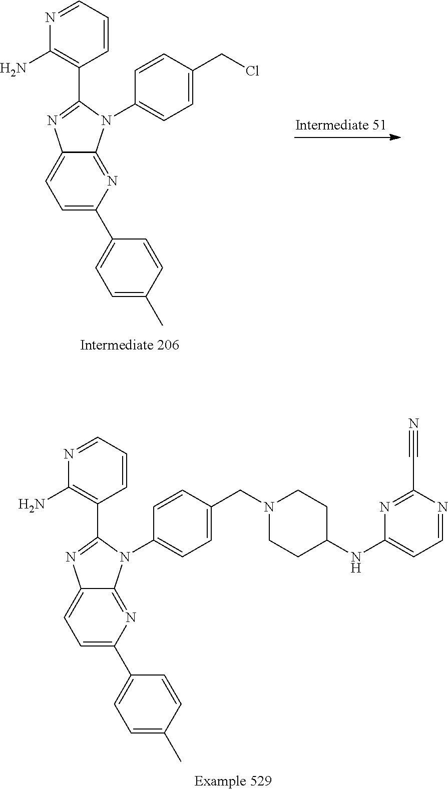 [SMILES: Cc1ccc(-c2ccc3nc(-c4cccnc4N)n(-c4ccc(CCl)cc4)c3n2)cc1 ; Cc1ccc(-c2ccc3nc(-c4cccnc4N)n(-c4ccc(CN5CCC(Nc6ccnc(C#N)n6)CC5)cc4)c3n2)cc1]

To a solution of Intermediate 206 (125 mg, 294 μmol) in DMF (3 mL) were added Intermediate 51 (77.8 mg, 383 μmol) and DIEA (205.2 μL, 1.2 mmol). The mixture was stirred at 80° C. for 4 hr. After purified by prep-HPLC (column: Phenomenex C18 75×30 mm×3 μm; mobile phase: [water (NH₃H₂O+NH₄HCO₃)-ACN]; gradient: 54%-84% B over 7 min), 4-((1-(4-(2-(2-aminopyridin-3-yl)-5-(p-tolyl)-3H-imidazo[4,5-b]pyridin-3-yl)benzyl)piperidin-4-yl)amino)pyrimidine-2-carbonitrile (Example 529, 125 mg, yield: 37%) was obtained as a light yellow solid. MS: m/z=593.3 [M+H]⁺. ¹H NMR (400 MHz, Dimethylsulfoxide-d₆) δ 8.24 (d, J=8.4 Hz, 1H), 8.11-8.05 (m, 2H), 8.00 (dd, J=4.8, 1.8 Hz, 1H), 7.94-7.92 (m, J=8.4 Hz, 3H), 7.52-7.42 (m, 4H), 7.27 (d, J=8.0 Hz, 2H), 7.15 (dd, J=7.6, 2.0 Hz, 1H), 7.04 (br s, 2H), 6.68 (d, J=6.0 Hz, 1H), 6.38 (dd, J=7.6, 4.8 Hz, 1H), 3.88-3.76 (m, 1H), 3.60 (s, 2H), 2.97-2.77 (m, 2H), 2.34 (s, 3H), 2.23-2.07 (m, 2H), 1.98-1.77 (m, 2H), 1.55-1.42 (m, 2H).

### Example 530: 4-((1-(4-(2-(2-Aminopyridin-3-yl)-5-(3-(trifluoromethyl)phenyl)-3H-imidazo[4,5-b]pyridin-3-yl)benzyl)piperidin-4-yl)amino)pyrimidine-2-carbonitrile

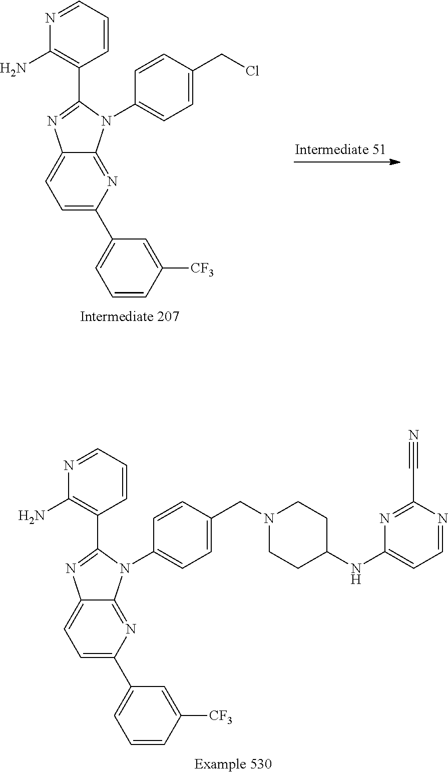 [SMILES: Nc1ncccc1-c1nc2ccc(-c3cccc(C(F)(F)F)c3)nc2n1-c1ccc(CCl)cc1 ; N#Cc1nccc(NC2CCN(Cc3ccc(-n4c(-c5cccnc5N)nc5ccc(-c6cccc(C(F)(F)F)c6)nc54)cc3)CC2)n1]

To a solution of Intermediate 207 (277.6 mg, 578.4 μmol) in DMF (4 mL) were added Intermediate 51 (188.9 mg, 929.2 μmol) and DIEA (369.5 mg, 2.9 mmol). The mixture was stirred at 80° C. for 4 hr under N₂. After purified by prep-HPLC (column: Phenomenex C18 75×30 mm×3 m; mobile phase: [water (NH₃H₂O+NH₄HCO₃)-ACN]; gradient: 56%-86% B over 7 min), 4-((1-(4-(2-(2-aminopyridin-3-yl)-5-(3-(trifluoromethyl)phenyl)-3H-imidazo[4,5-b]pyridin-3-yl)benzyl)piperidin-4-yl)amino)pyrimidine-2-carbonitrile (Example 530, 161.3 mg, yield: 34.9%) was obtained as a light yellow solid. MS: m/z=647.3 [M+H]⁺. ¹H NMR (400 MHz, Dimethylsulfoxide-d₆) δ 8.38-8.30 (m, 3H), 8.14 (d, J=8.4 Hz, 1H), 8.10-8.04 (m, 2H), 8.01 (dd, J=4.8, 2.0 Hz, 1H), 7.77-7.68 (m, 2H), 7.52-7.44 (m, 4H), 7.19 (dd, J=7.6, 1.6 Hz, 1H), 7.07 (s, 2H), 6.68 (br d, J=6.4 Hz, 1H), 6.39 (dd, J=8.0, 4.8 Hz, 1H), 3.81 (br s, 1H), 3.60 (s, 2H), 2.83-2.81 (m, 2H), 2.20-2.09 (m, 2H), 1.94-1.80 (m, 2H), 1.54-1.45 (m, 2H). ¹⁹F NMR (400 MHz, Dimethylsulfoxide-d₆) δ −61.256.

### Example 531: Isopropyl 2-(2-aminopyridin-3-yl)-3-(4-((4-((2-cyanopyrimidin-4-yl)amino)piperidin-1-yl)methyl)phenyl)-3H-imidazo[4,5-b]pyridine-5-carboxylate

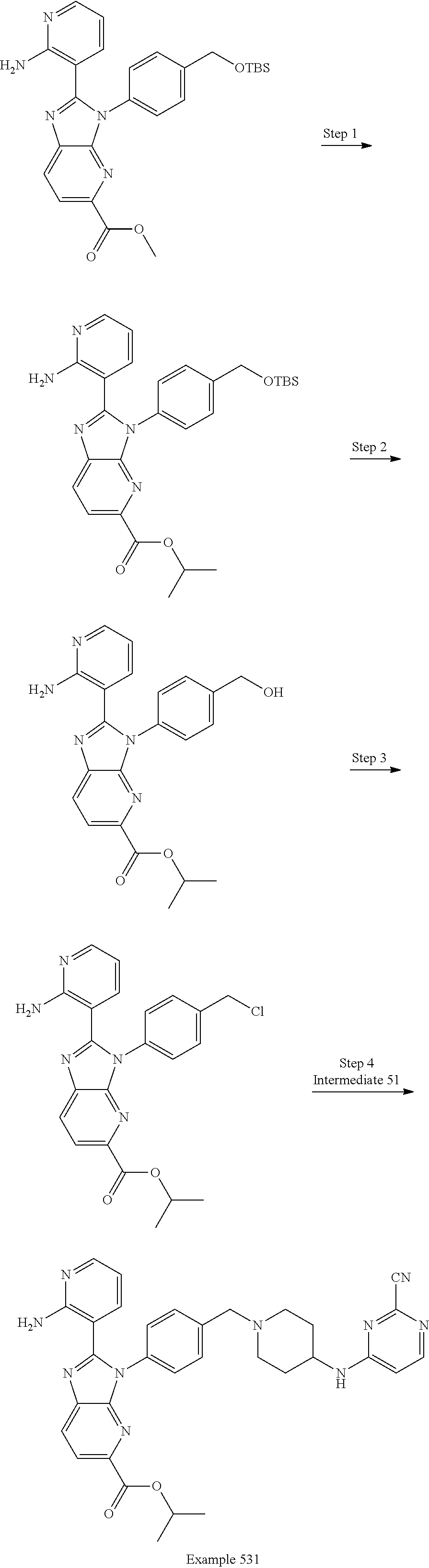 [SMILES: COC(=O)c1ccc2nc(-c3cccnc3N)n(-c3ccc(CO[Si](C)(C)C(C)(C)C)cc3)c2n1 ; CC(C)OC(=O)c1ccc2nc(-c3cccnc3N)n(-c3ccc(CO[Si](C)(C)C(C)(C)C)cc3)c2n1 ; CC(C)OC(=O)c1ccc2nc(-c3cccnc3N)n(-c3ccc(CO)cc3)c2n1 ; CC(C)OC(=O)c1ccc2nc(-c3cccnc3N)n(-c3ccc(CCl)cc3)c2n1 ; CC(C)OC(=O)c1ccc2nc(-c3cccnc3N)n(-c3ccc(CN4CCC(Nc5ccnc(C#N)n5)CC4)cc3)c2n1]

### Step 1: Isopropyl 2-(2-aminopyridin-3-yl)-3-(4-(((tert-butyldimethylsilyl)oxy)methyl)phenyl)-3H-imidazo[4,5-b]pyridine-5-carboxylate

To a solution of methyl 2-(2-aminopyridin-3-yl)-3-(4-(((tert-butyldimethylsilyl)oxy)methyl)phenyl)-3H-imidazo[4,5-b]pyridine-5-carboxylate (refer to Example 508 for detail procedures, 280 mg, 572 μmol) in i-PrOH (10 mL) was added i-PrOLi (45.3 mg, 686 μmol). The reaction mixture was stirred at 65° C. for 0.5 hr. After cooling to 25° C., water (20 mL) was added. The mixture was extracted with EtOAc (20 mL×3). The combined organic layers were washed with brine (20 mL×2), dried over anhydrous Na₂SO₄, filtered, and concentrated under reduced pressure. After purified by silica gel flash chromatography (MeOH in CH₂Cl₂=0-20%), isopropyl 2-(2-aminopyridin-3-yl)-3-(4-(((tert-butyldimethylsilyl)oxy)methyl)phenyl)-3H-imidazo[4,5-b]pyridine-5-carboxylate (160 mg, yield: 54.1%) was obtained as a yellow solid. MS: m/z=518.3 [M+H]⁺.

### Step 2. Isopropyl 2-(2-aminopyridin-3-yl)-3-(4-(hydroxymethyl)phenyl)-3H-imidazo[4,5-b]pyridine-5-carboxylate

To a solution of isopropyl 2-(2-aminopyridin-3-yl)-3-(4-(((tert-butyldimethylsilyl)oxy)methyl)phenyl)-3H-imidazo[4,5-b]pyridine-5-carboxylate (150 mg, 290 μmol) in THF (5 mL) was added TBAF (1 M in THF, 435 μL). The reaction mixture was stirred at 20° C. for 1 hr. Water (20 mL) was added. The mixture was extracted with EtOAc (20 mL×3). The combined organic layers were washed with brine (20 mL×2), dried over anhydrous Na₂SO₄, filtered, and concentrated under reduced pressure. Isopropyl 2-(2-aminopyridin-3-yl)-3-(4-(hydroxymethyl)phenyl)-3H-imidazo[4,5-b]pyridine-5-carboxylate (117 mg, crude) was obtained as a brown solid, which was used directly into the next step. MS: m/z=404.2 [M+H]⁺.

### Step 3: Isopropyl 2-(2-aminopyridin-3-yl)-3-(4-(chloromethyl)phenyl)-3H-imidazo[4,5-b]pyridine-5-carboxylate

To a solution of isopropyl 2-(2-aminopyridin-3-yl)-3-(4-(hydroxymethyl)phenyl)-3H-imidazo[4,5-b]pyridine-5-carboxylate (107 mg, 265 μmol) in CH₂Cl₂ (3 mL) was added SOCl₂ (94.6 mg, 795 μmol). The reaction mixture was stirred at 20° C. for 2 hr. The reaction mixture was concentrated to give isopropyl 2-(2-aminopyridin-3-yl)-3-(4-(chloromethyl)phenyl)-3H-imidazo[4,5-b]pyridine-5-carboxylate (112 mg, 265 μmol, crude) as a yellow solid, which was used directly into the next step. MS: m/z=422.1 [M+H]⁺.

### Step 4: Isopropyl 2-(2-aminopyridin-3-yl)-3-(4-((4-((2-cyanopyrimidin-4-yl)amino)piperidin-1-yl)methyl)phenyl)-3H-imidazo[4,5-b]pyridine-5-carboxylate

To a solution of isopropyl 2-(2-aminopyridin-3-yl)-3-(4-(chloromethyl)phenyl)-3H-imidazo[4,5-b]pyridine-5-carboxylate (112 mg, 265 μmol) and Intermediate 51 (59.2 mg, 291 μmol) in DMF (3 mL) were added NaI (7.94 mg, 53.0 μmol) and K₂CO₃ (110 mg, 795 μmol) at 20° C. The reaction mixture was stirred at 20° C. for 12 hr. The reaction mixture was diluted with H₂O (20 ml) and extracted with EtOAc (20 ml). The organic layer was separated, dried over anhydrous Na₂SO₄ and concentrated. After purified by prep-HPLC (column: C18 150×30 mm; mobile phase: [water (NH₃H₂O+NH₄HCO₃)-ACN]; gradient: 50%-80% B over 7 min), isopropyl 2-(2-aminopyridin-3-yl)-3-(4-((4-((2-cyanopyrimidin-4-yl)amino)piperidin-1-yl)methyl)phenyl)-3H-imidazo[4,5-b]pyridine-5-carboxylate (Example 531, 28.4 mg, yield: 18.2% for 3 steps) was obtained as a yellow solid. MS: m/z=589.3 [M+H]⁺. ¹H NMR (400 MHz, Dimethylsulfoxide-d₆) δ 8.32 (d, J=8.4 Hz, 1H), 8.11-8.03 (m, 3H), 8.01 (dd, J=4.4, 1.6 Hz, 1H), 7.50-7.40 (m, 4H), 7.18 (dd, J=7.6, 1.6 Hz, 1H), 7.00 (s, 2H), 6.67 (d, J=6.0 Hz, 1H), 6.38 (dd, J=8.0, 5.2 Hz, 1H), 5.17-5.08 (m, 1H), 3.82 (br.s, 1H), 3.58 (s, 2H), 2.87-2.78 (m, 2H), 2.21-2.11 (m, 2H), 1.93-1.84 (m, 2H), 1.56-1.43 (m, 2H), 1.32 (d, J=6.4 Hz, 6H).

### Example 532: 4-((1-(4-(2-(2-Aminopyridin-3-yl)-1H-benzo[d]imidazol-1-yl)benzyl)piperidin-4-yl)amino)pyrimidine-2-carbonitrile

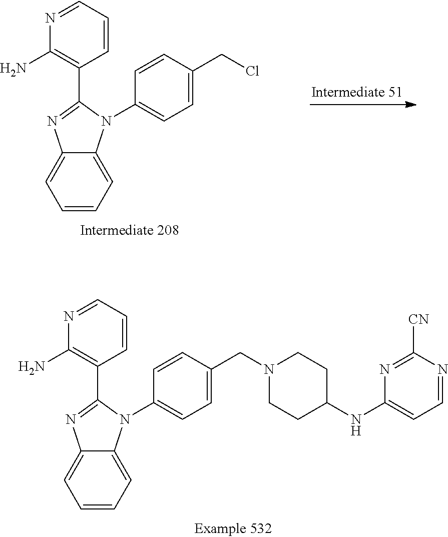 [SMILES: Nc1ncccc1-c1nc2ccccc2n1-c1ccc(CCl)cc1 ; N#Cc1nccc(NC2CCN(Cc3ccc(-n4c(-c5cccnc5N)nc5ccccc54)cc3)CC2)n1]

To a solution of Intermediate 208 (180 mg, 537 μmol) and Intermediate 51 (131 mg, 645 μmol) in DMF (5 mL) were added NaI (16.1 mg, 107 μmol) and K₂CO₃ (223 mg, 1.61 mmol, 300 μL) at 20° C. The reaction mixture was stirred at 20° C. for 12 hr. The reaction mixture was diluted with H₂O (20 ml) and extracted with EtOAc (20 mL). The organic layer was separated, dried over anhydrous Na₂SO₄ and concentrated. After purified by prep-HPLC (column: C18 150×30 mm; mobile phase: [water (NH₃H₂O+NH₄HCO₃)-ACN]; gradient: 57%-77% B over 7 min), 4-((1-(4-(2-(2-aminopyridin-3-yl)-1H-benzo[d]imidazol-1-yl)benzyl)piperidin-4-yl)amino)pyrimidine-2-carbonitrile (Example 532, 83.1 mg, yield: 30.8% for 2 steps) was obtained as a white solid. MS: m/z=502.3 [M+H]⁺. ¹H NMR (400 MHz, Dimethylsulfoxide-d₆) δ 8.13-8.02 (m, 2H), 7.96 (dd, J=4.8, 1.6 Hz, 1H), 7.80 (d, J=7.2 Hz, 1H), 7.49 (d, J=8.4 Hz, 2H), 7.37 (d, J=8.4 Hz, 2H), 7.35-7.27 (m, 2H), 7.20 (d, J=7.2 Hz, 1H), 7.11-7.06 (m, 3H), 6.67 (d, J=6.0 Hz, 1H), 6.34 (dd, J=7.6, 4.8 Hz, 1H), 3.81 (s, 1H), 3.59 (s, 2H), 2.87-2.77 (m, 2H), 2.19-2.10 (m, 2H), 1.94-1.83 (m, 2H), 1.57-1.43 (m, 2H).

### Example 533: 4-((1-(4-(2-(2-Aminopyridin-3-yl)-1H-imidazo[4,5-b]pyridin-1-yl)benzyl)piperidin-4-yl)amino)pyrimidine-2-carbonitrile

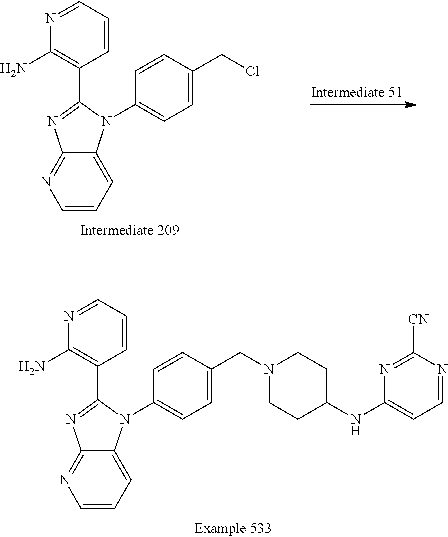 [SMILES: Nc1ncccc1-c1nc2ncccc2n1-c1ccc(CCl)cc1 ; N#Cc1nccc(NC2CCN(Cc3ccc(-n4c(-c5cccnc5N)nc5ncccc54)cc3)CC2)n1]

To a solution of Intermediate 209 (106 mg, 315 μmol) and Intermediate 51 (70.5 mg, 347 μmol) in DMF (3 mL) were added K₂CO₃ (131 mg, 945 μmol) and NaI (9.45 mg, 63.0 μmol) at 20° C. The reaction mixture was stirred at 20° C. for 12 hr. The reaction mixture was diluted with H₂O (20 mL) and extracted with EtOAc (20 mL). The organic layer was separated, dried over anhydrous Na₂SO₄, and concentrated under reduced pressure. After purified by prep-HPLC (column: C18 150×30 mm, mobile phase: [water (NH₃H₂O+NH₄HCO₃)-ACN]; gradient: 40%-70% B over 7 min), 4-((1-(4-(2-(2-aminopyridin-3-yl)-1H-imidazo[4,5-b]pyridin-1-yl)benzyl)piperidin-4-yl)amino)pyrimidine-2-carbonitrile (Example 533, 40.5 mg, yield: 25.6%) was obtained as an off-white solid. MS: m/z=503.4 [M+H]⁺. ¹H NMR (400 MHz, Dimethylsulfoxide-d₆) δ 8.51 (dd, J=4.8, 1.2 Hz, 1H), 8.12-8.03 (m, 2H), 8.00 (dd, J=4.8, 1.6 Hz, 1H), 7.65 (d, J=8.0 Hz, 1H), 7.52-7.47 (m, 2H), 7.44-7.39 (m, 2H), 7.32 (dd, J=8.0, 4.8 Hz, 1H), 7.15 (dd, J=7.6, 1.2 Hz, 1H), 7.05 (s, 2H), 6.67 (d, J=6.4 Hz, 1H), 6.38 (dd, J=7.6, 4.8 Hz, 1H), 3.81 (s, 1H), 3.59 (s, 2H), 2.86-2.77 (m, 2H), 2.22-2.07 (m, 2H), 1.93-1.83 (m, 2H), 1.56-1.43 (m, 2H).

### Example 534: 4-((1-(4-(2-(2-Aminopyridin-3-yl)-5-(6-methylpyridin-3-yl)-3H-imidazo[4,5-b]pyridin-3-yl)benzyl)piperidin-4-yl)amino)pyrimidine-2-carbonitrile

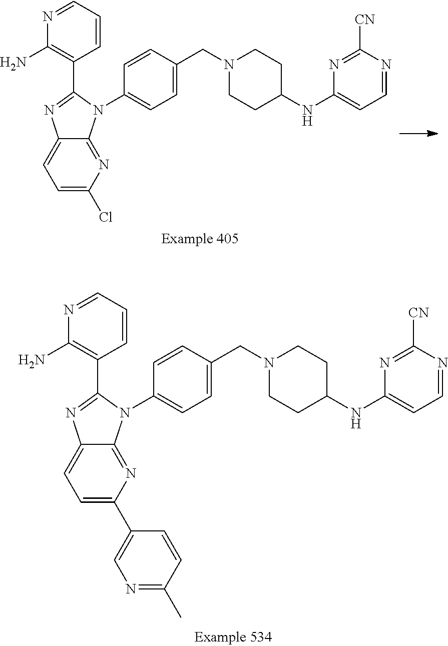 [SMILES: N#Cc1nccc(NC2CCN(Cc3ccc(-n4c(-c5cccnc5N)nc5ccc(Cl)nc54)cc3)CC2)n1 ; Cc1ccc(-c2ccc3nc(-c4cccnc4N)n(-c4ccc(CN5CCC(Nc6ccnc(C#N)n6)CC5)cc4)c3n2)cn1]

To a solution of Example 405 (200 mg, 372 μmol) and (6-methyl-3-pyridyl)boronic acid (56.1 mg, 410 μmol) in 1,4-dioxane (5 mL) and H₂O (1 mL) were added Pd(dppf)Cl₂ (27.3 mg, 37.2 μmol) and Cs₂CO₃ (364 mg, 1.12 mmol) at 25° C. The mixture was degassed and purged with N₂ three times, and stirred at 100° C. for 1 hr. The mixture was quenched with H₂O (50 mL) and extracted with EtOAc (50 mL×3). The combined organic layers were washed with brine (50 mL), dried over Na₂SO₄, filtered, and concentrated. After purified by silica gel flash chromatography (MeOH in CH₂Cl₂=0-10%), 4-((1-(4-(2-(2-aminopyridin-3-yl)-5-(6-methylpyridin-3-yl)-3H-imidazo[4,5-b]pyridin-3-yl)benzyl)piperidin-4-yl)amino)pyrimidine-2-carbonitrile (Example 534, 87.7 mg, yield: 40%) was obtained as a yellow solid. MS: m/z=594.1 [M+H]⁺. 1H NMR (400 MHz, Dimethylsulfoxide-d₆) 9.10 (d, J=2.0 Hz, 1H), 8.34-8.22 (m, 2H), 8.13-7.97 (m, 4H), 7.53-7.43 (m, 4H), 7.34 (d, J=8.4 Hz, 1H), 7.16 (dd, J=7.6, 1.6 Hz, 1H), 7.04 (s, 2H), 6.68 (d, J=6.4 Hz, 1H), 6.39 (dd, J=7.6, 4.8 Hz, 1H), 3.90-3.75 (m, 1H), 3.59 (s, 2H), 2.90-2.76 (m, 2H), 2.50 (s, 3H), 2.23-2.11 (m, 2H), 1.96-1.84 (m, 2H), 1.58-1.40 (m, 2H).

### Example 535: 4-((1-(4-(2-(2-Aminopyridin-3-yl)-5-(difluoromethoxy)-3H-imidazo[4,5-b]pyridin-3-yl)benzyl)piperidin-4-yl)amino)pyrimidine-2-carbonitrile

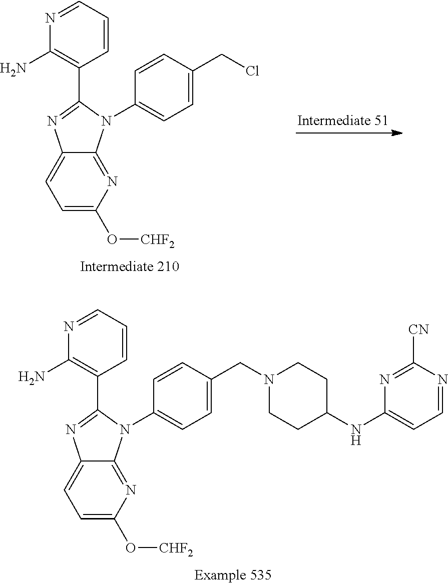 [SMILES: Nc1ncccc1-c1nc2ccc(OC(F)F)nc2n1-c1ccc(CCl)cc1 ; N#Cc1nccc(NC2CCN(Cc3ccc(-n4c(-c5cccnc5N)nc5ccc(OC(F)F)nc54)cc3)CC2)n1]

To a solution of Intermediate 210 (123 mg, 306 μmol) and Intermediate 51 (68.3 mg, 336 μmol) in DMF (5 mL) were added NaI (9.16 mg, 61.1 μmol) and K₂CO₃ (127 mg, 917 μmol) at 20° C. The reaction mixture was stirred at 20° C. for 12 hr. The mixture was diluted with H₂O (20 mL) and extracted with EtOAc (20 mL×3). The combined organic layers were washed with brine (20 mL×2), dried over anhydrous Na₂SO₄, filtered, and concentrated under reduced pressure. After purified by prep-HPLC (column: Phenomenex C18 80×30 mm×5 μm; mobile phase: [water (NH₃H₂O+NH₄HCO₃)-ACN]; gradient: 52%-82% B over 7 min), 4-((1-(4-(2-(2-aminopyridin-3-yl)-5-(difluoromethoxy)-3H-imidazo[4,5-h]pyridin-3-yl)benzyl)piperidin-4-yl)amino)pyrimidine-2-carbonitrile (Example 535, 40.3 mg, yield: 23.2% for 3 steps) was obtained as an off-white solid. MS: m/z=569.3 [M+H]⁺. ¹H NMR (400 MHz, Dimethylsulfoxide-d₆) δ 8.33 (d, J=8.4 Hz, 1H), 8.07 (dd, J=9.6, 6.0 Hz, 2H), 7.98 (dd, J=4.8, 1.6 Hz, 1H), 7.75-7.51 (m, 1H), 7.48-7.42 (m, 2H), 7.39-7.35 (m, 2H), 7.13-7.05 (m, 2H), 6.89 (s, 2H), 6.67 (d, J=6.0 Hz, 1H), 6.37 (dd, J=7.6, 4.8 Hz, 1H), 3.84-3.78 (m, 1H), 3.57 (s, 2H), 2.87-2.77 (m, 2H), 2.21-2.08 (m, 2H), 1.95-1.82 (m, 2H), 1.57-1.42 (m, 2H). ¹⁹F NMR (400 MHz, Dimethylsulfoxide-d₆) δ −86.292.

### Example 536: 4-((1-(4-(2-(2-Aminopyridin-3-yl)-5-(4-methoxypyridin-3-yl)-3H-imidazo[4,5-b]pyridin-3-yl)benzyl)piperidin-4-yl)amino)pyrimidine-2-carbonitrile

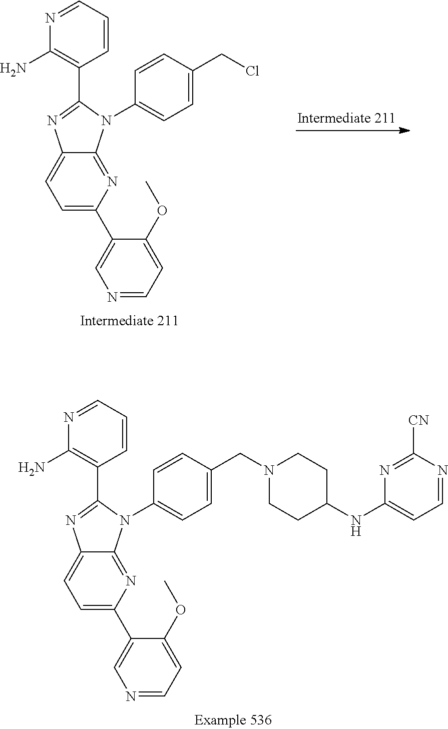 [SMILES: COc1ccncc1-c1ccc2nc(-c3cccnc3N)n(-c3ccc(CCl)cc3)c2n1 ; COc1ccncc1-c1ccc2nc(-c3cccnc3N)n(-c3ccc(CN4CCC(Nc5ccnc(C#N)n5)CC4)cc3)c2n1]

To a solution of Intermediate 211 (190 mg, 429 μmol) in DMF (3 mL) were added DIEA (222 mg, 1.72 mmol) and Intermediate 51 (113 mg, 558 μmol). The mixture was stirred at 80° C. for 2 hr. After purified by prep-HPLC(column: Phenomenex C18 80×30 mm×5 μm; mobile phase: [water (NH₃H₂O+NH₄HCO₃)-ACN]; gradient: 38%-68% B over 7 min), 4-((1-(4-(2-(2-aminopyridin-3-yl)-5-(4-methoxypyridin-3-yl)-3H-imidazo[4,5-b]pyridin-3-yl)benzyl)piperidin-4-yl)amino)pyrimidine-2-carbonitrile (Example 536, 44.6 mg, yield: 17.1%) was obtained as a yellow solid. MS: m/z=610.3 [M+H]⁺. ¹H NMR (400 MHz, Dimethylsulfoxide-d₆) δ 8.66 (s, 1H), 8.46 (d, J=6.4 Hz, 1H), 8.25 (d, J=8.4 Hz, 1H), 8.10-8.03 (m, 2H), 8.02-7.96 (dd, J=4.8, 1.6 Hz, 1H), 7.85 (d, J=8.4 Hz, 1H), 7.46-7.42 (m, 4H), 7.20 (d, J=6.0 Hz, 1H), 7.17-7.13 (dd, J=7.6, 1.6 Hz, 1H), 7.03 (s, 2H), 6.66 (d, J=6.0 Hz, 1H), 6.41-6.35 (m, 1H), 3.92 (s, 3H), 3.85 (br.s, 1H), 3.56 (s, 2H), 2.80-2.75 (m, 2H), 2.30-2.10 (m, 2H), 1.89 (d, J=10.4 Hz, 2H), 1.60-1.40 (m, 2H).

### Example 537 & 538: 4-((1-(4-(2-(2-Aminopyridin-3-yl)-5-(2-methoxypyridin-3-yl)-3H-imidazo[4,5-b]pyridin-3-yl)benzyl)piperidin-4-yl)amino)pyrimidine-2-carbonitrile & 4-((1-(4-(2-(2-Aminopyridin-3-yl)-5-(2-hydroxypyridin-3-yl)-3H-imidazo[4,5-b]pyridin-3-yl)benzyl)piperidin-4-yl)amino)pyrimidine-2-carbonitrile

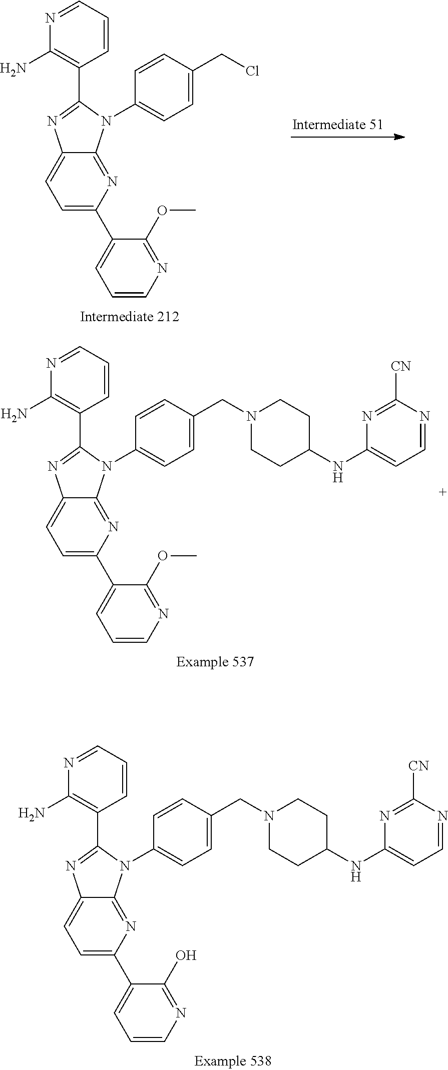 [SMILES: COc1ncccc1-c1ccc2nc(-c3cccnc3N)n(-c3ccc(CCl)cc3)c2n1 ; COc1ncccc1-c1ccc2nc(-c3cccnc3N)n(-c3ccc(CN4CCC(Nc5ccnc(C#N)n5)CC4)cc3)c2n1 ; N#Cc1nccc(NC2CCN(Cc3ccc(-n4c(-c5cccnc5N)nc5ccc(-c6cccnc6O)nc54)cc3)CC2)n1]

To a solution of Intermediate 212 (350 mg, 790 μmol) and Intermediate 51 (177 mg, 869 μmol) in DMF (12 mL) were added NaI (23.7 mg, 158 μmol) and K₂CO₃ (328 mg, 2.37 mmol) at 20° C. The reaction mixture was stirred at 80° C. for 2 hr. The mixture was diluted with H₂O (30 mL) and extracted with EtOAc (50 mL×3). The combined organic layers were washed with brine (50 mL×2), dried over anhydrous Na₂SO₄, filtered, and concentrated under reduced pressure. After purified by prep-HPLC (column: C18 150-30 mm; mobile phase: [water (NH₃H₂O+NH₄HCO₃)-ACN]; gradient: 50%-80% B over 7 min), 4-((1-(4-(2-(2-aminopyridin-3-yl)-5-(2-methoxypyridin-3-yl)-3H-imidazo[4,5-b]pyridin-3-yl)benzyl)piperidin-4-yl)amino)pyrimidine-2-carbonitrile (Example 537, 12.7 mg, yield: 2.6% for 4 steps) was obtained as an off-white solid. MS: m: =610.4 [M+H]⁺. ¹H NMR (400 MHz, Dimethylsulfoxide-d₆) δ 8.27-8.21 (m, 2H), 8.12-8.03 (m, 3H), 8.00 (d, J=8.0 Hz, 2H), 7.48-7.42 (m, 4H), 7.16-7.09 (m, 2H), 7.02 (s, 2H), 6.67 (d, J=6.0 Hz, 1H), 6.38 (dd, J=8.0, 5.2 Hz, 1H), 3.96 (s, 3H), 3.87-3.73 (m, 1H), 3.57 (s, 2H), 2.87-2.76 (m, 2H), 2.20-2.09 (m, 2H), 1.93-1.80 (m, 2H), 1.57-1.41 (m, 2H). 4-((1-(4-(2-(2-aminopyridin-3-yl)-5-(2-hydroxypyridin-3-yl)-3H-imidazo[4,5-b]pyridin-3-yl)benzyl)piperidin-4-yl)amino)pyrimidine-2-carbonitrile (Example 538, 53.8 mg, yield: 11.4% for 4 steps) was obtained as an off-white solid. MS: m/z=596.1 [M+H]⁺. ¹H NMR (400 MHz, Dimethylsulfoxide-d₆) δ 11.99 (s, 1H), 8.68 (d, J=8.4 Hz, 1H), 8.25-8.17 (m, 2H), 8.14-8.03 (m, 2H), 7.99 (d, J=3.6 Hz, 1H), 7.50-7.41 (m, 5H), 7.13 (d, J=7.6 Hz, 1H), 7.05 (s, 2H), 6.67 (d, J=6.0 Hz, 1H), 6.40-6.33 (m, 2H), 3.93-3.73 (m, 1H), 3.59 (s, 2H), 2.87-2.78 (m, 2H), 2.22-2.09 (m, 2H), 1.94-1.83 (m, 2H), 1.58-1.42 (m, 2H).

### Example 539: 4-((1-(4-(2-(2-Aminopyridin-3-yl)-5-(1,3-dimethyl-1H-pyrazol-4-yl)-3H-imidazo[4,5-b]pyridin-3-yl)benzyl)piperidin-4-yl)amino)pyrimidine-2-carbonitrile

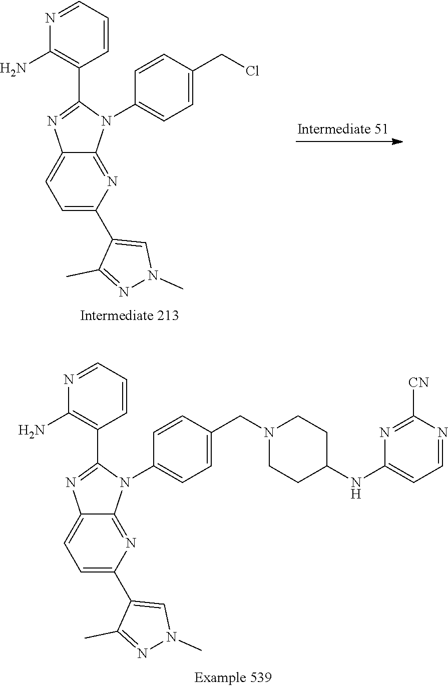 [SMILES: Cc1nn(C)cc1-c1ccc2nc(-c3cccnc3N)n(-c3ccc(CCl)cc3)c2n1 ; Cc1nn(C)cc1-c1ccc2nc(-c3cccnc3N)n(-c3ccc(CN4CCC(Nc5ccnc(C#N)n5)CC4)cc3)c2n1]

To a solution of Intermediate 213 (440 mg, 1.02 mmol) and Intermediate 51 (229 mg, 1.13 mmol) in DMF (16 mL) were added K₂CO₃ (424 mg, 3.07 mmol) and NaI (30.7 mg, 205 μmol) at 20° C. The reaction mixture was stirred at 80° C. for 2 hr. The mixture was diluted with H₂O (30 mL) and extracted with EtOAc (50 mL×3). The combined organic layers were washed with brine (50 mL×2), dried over anhydrous Na₂SO₄, filtered, and concentrated under reduced pressure. After purified by prep-HPLC (column: C18 150×30 mm; mobile phase: [water (NH₃H₂O+NH₄HCO₃)-ACN]; gradient: 45%-75% B over 7 min), 4-((1-(4-(2-(2-aminopyridin-3-yl)-5-(1,3-dimethyl-1H-pyrazol-4-yl)-3H-imidazo[4,5-b]pyridin-3-yl)benzyl)piperidin-4-yl)amino)pyrimidine-2-carbonitrile (Example 539, 139.6 mg, yield: 23% for 4 steps) was obtained as an off-white solid. MS: m/z=597.1 [M+H]⁺. ¹H NMR (400 MHz, Dimethylsulfoxide-d₆) δ 8.18 (s, 1H), 8.14 (d, J=8.4 Hz, 1H), 8.10-8.04 (m, 2H), 7.99 (dd, J=4.8, 2.0 Hz, 1H), 7.57 (d, J=8.4 Hz, 1H), 7.48-7.44 (m, 2H), 7.43-7.39 (m, 2H), 7.19 (dd, J=7.6, 1.6 Hz, 1H), 7.10 (s, 2H), 6.67 (d, J=6.0 Hz, 1H), 6.38 (dd, J=7.6, 4.8 Hz, 1H), 3.83-3.75 (m, 4H), 3.57 (s, 2H), 2.83-2.77 (m, 2H), 2.27 (s, 3H), 2.20-2.03 (m, 2H), 1.92-1.84 (m, 2H), 1.53-1.43 (m, 2H).

### Example 540: 4-((1-(4-(2-(2-Aminopyridin-3-yl)-1H-imidazo[4,5-c]pyridin-1-yl)benzyl)piperidin-4-yl)amino)pyrimidine-2-carbonitrile

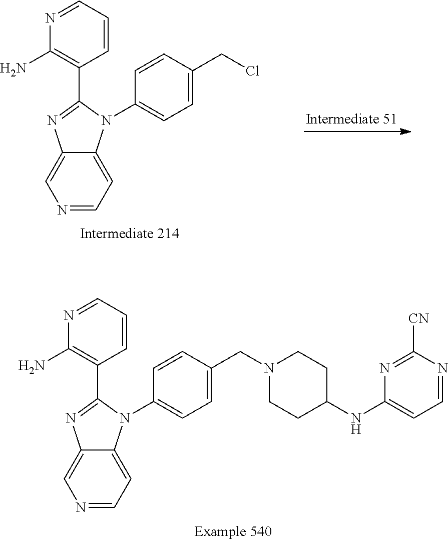 [SMILES: Nc1ncccc1-c1nc2cnccc2n1-c1ccc(CCl)cc1 ; N#Cc1nccc(NC2CCN(Cc3ccc(-n4c(-c5cccnc5N)nc5cnccc54)cc3)CC2)n1]

To a solution of Intermediate 214 (100 mg, 298 μmol) and Intermediate 51 (66.6 mg, 328 μmol) in DMF (3 mL) were added K₂CO₃ (123 mg, 893 μmol) and NaI (4.46 mg, 29.8 μmol) at 25° C. This mixture was stirred at 25° C. for 12 hr. The mixture was quenched with water (20 ml) and extracted with EtOAc (20 mL×3). The combined organic layers were washed with brine (20 mL), dried over Na₂SO₄, filtered and concentrated. After purified by prep-HPLC (column: C18 150×30 mm; mobile phase: [water (NH₃H₂O+NH₄HCO₃)-ACN]; gradient: 38%-68% B over 7 min), 4-((1-(4-(2-(2-aminopyridin-3-yl)-1H-imidazo[4,5-c]pyridin-1-yl)benzyl)piperidin-4-yl)amino)pyrimidine-2-carbonitrile (Example 540, 38.0 mg, yield: 38%) was obtained as an off-white solid. MS: m/z=503.2 [M+H]⁺. ¹H NMR (400 MHz, Dimethylsulfoxide-d₆) δ 9.09 (s, 1H), 8.38 (d, J=5.6 Hz, 1H), 8.13-8.02 (m, 2H), 7.99 (dd, J=4.8, 2.0 Hz, 1H), 7.49 (d, J=8.4 Hz, 2H), 7.41 (d, J=8.0 Hz, 2H), 7.26 (d, J=5.2 Hz, 1H), 7.16 (dd, J=7.6, 1.6 Hz, 1H), 6.96 (s, 2H), 6.67 (d, J=6.0 Hz, 1H), 6.38 (dd, J=7.6, 4.8 Hz, 1H), 3.90-3.74 (m, 1H), 3.58 (s, 2H), 2.87-2.77 (m, 2H), 2.23-2.07 (m, 2H), 1.95-1.81 (m, 2H), 1.57-1.41 (m, 2H).

### Example 541: 4-((1-(4-(2-(2-Aminopyridin-3-yl)-5-(4-fluorophenyl)-3H-imidazo[4,5-b]pyridin-3-yl)benzyl)piperidin-4-yl)(methyl)amino)pyrimidine-2-carbonitrile

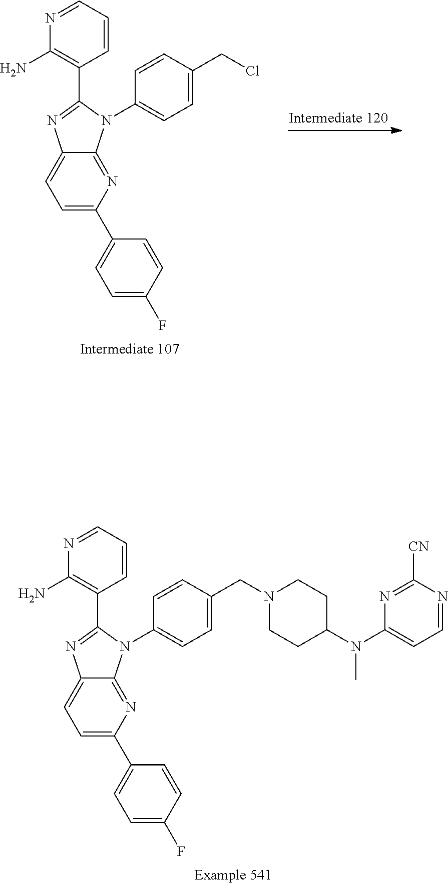 [SMILES: Nc1ncccc1-c1nc2ccc(-c3ccc(F)cc3)nc2n1-c1ccc(CCl)cc1 ; CN(c1ccnc(C#N)n1)C1CCN(Cc2ccc(-n3c(-c4cccnc4N)nc4ccc(-c5ccc(F)cc5)nc43)cc2)CC1]

To a solution of Intermediate 107 (100 mg, 233 μmol) in DMF (3 mL) were added Intermediate 120 (61 mg, 280 μmol), NaI (35 mg, 233 μmol) and K₂CO₃ (129 mg, 931 μmol) at 25° C. The mixture was stirred at 50° C. for 1 hr. The reaction mixture was poured into H₂O (50 mL) and extracted with CH₂Cl₂ (20 mL×2). The combined organic layers were washed with brine (40 mL), dried over anhydrous Na₂SO₄, filtered and concentrated. After purified by silica gel flash chromatography (Eluent of 0˜5% MeOH in CH₂Cl₂), 4-((1-(4-(2-(2-aminopyridin-3-yl)-5-(4-fluorophenyl)-3H-imidazo[4,5-b]pyridin-3-yl)benzyl)piperidin-4-yl)(methyl)amino)pyrimidine-2-carbonitrile (Example 541, 25.8 mg, yield: 17%) was obtained as an off white solid. MS: m/z=611.3 [M+H]⁺. ¹H NMR (400 MHz, Dimethylsulfoxide-d₆) δ 8.27 (d, J=8.4 Hz, 1H), 8.24 (d, J=6.0 Hz, 1H), 8.10-8.05 (m, 2H), 8.01-7.96 (m, 2H), 7.52-7.43 (m, 4H), 7.30 (t, J=8.8 Hz, 2H), 7.15 (dd, J=8.0, 1.6 Hz, 1H), 7.02 (s, 3H), 6.38 (dd, J=7.6, 4.8 Hz, 1H), 3.62 (s, 2H), 2.94 (s, 6H), 2.22-2.13 (m, 2H), 1.89-1.80 (m, 2H), 1.65-1.56 (m, 2H). ¹⁹F NMR (400 MHz, Dimethylsulfoxide-d₆) δ −113.59.

### Example 542: 4-((1-(4-(2-(2-Aminopyridin-3-yl)-5-(6-(difluoromethoxy)pyridin-3-yl)-3H-imidazo[4,5-b]pyridin-3-yl)benzyl)piperidin-4-yl)amino)pyrimidine-2-carbonitrile

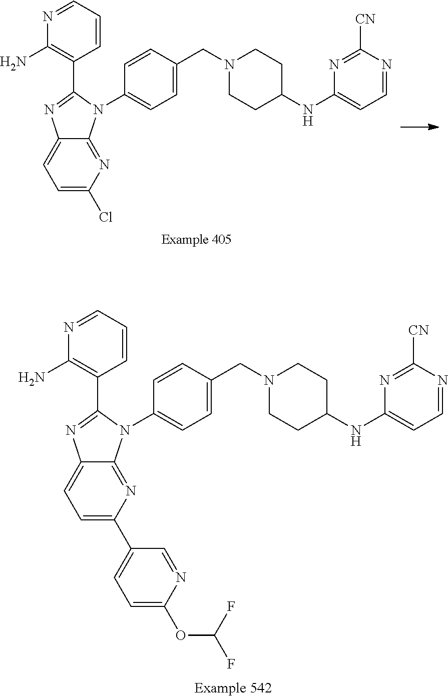 [SMILES: N#Cc1nccc(NC2CCN(Cc3ccc(-n4c(-c5cccnc5N)nc5ccc(Cl)nc54)cc3)CC2)n1 ; N#Cc1nccc(NC2CCN(Cc3ccc(-n4c(-c5cccnc5N)nc5ccc(-c6ccc(OC(F)F)nc6)nc54)cc3)CC2)n1]

A mixture of (6-(difluoromethoxy)pyridin-3-yl)boronic acid (101 mg, 536 μmol), Example 405 (240 mg, 447 μmol), Cs₂CO₃ (437 mg, 1.3 mmol), Pd(dppf)Cl₂ (32.7 mg, 45 μmol) in H₂O (1 mL) and 1,4-dioxane (5 mL) was degassed and purged with N₂ three times. The mixture was stirred at 100° C. for 1 hr under N₂ atmosphere. The reaction mixture was diluted with H₂O (30 mL) and extracted with EtOAc (20 mL×3). The combined organic layers were washed with brine (30 mL), dried over Na₂SO₄, filtered and concentrated under reduced pressure. After purified by prep-HPLC (column: Phenomenex C18 150×25 mm×10 μm; mobile phase: [water (NH₄HCO₃)-ACN]; gradient: 47%-77% B over 11 min), 4-((1-(4-(2-(2-aminopyridin-3-yl)-5-(6-(difluoromethoxy)pyridin-3-yl)-3H-imidazo[4,5-b]pyridin-3-yl)benzyl)piperidin-4-yl)amino)pyrimidine-2-carbonitrile (Example 542, 68.7 mg, yield: 24%) was obtained as an off white solid. MS: m/z=646.3 [M+H]⁺. ¹H NMR (400 MHz, Dimethysulfoxide-d₆) δ8.92 (d, J=2.4 Hz, 1H), 8.50 (dd, J=8.8, 2.4 Hz, 1H), 8.31 (d, J=8.4 Hz, 1H), 8.15-7.96 (m, 4H), 7.95-7.77 (m, 1H), 7.52-7.43 (m, 4H), 7.22-7.13 (m, 2H), 7.04 (br s, 2H), 6.67 (d, J=6.0 Hz, 1H), 6.39 (dd, J=7.6, 4.8 Hz, 1H), 3.81 (dd, J=6.8, 2.4 Hz, 1H), 3.59 (s, 2H), 2.83 (d, J=10.8 Hz, 2H), 2.16 (t, 1=10.8 Hz, 2H), 1.89 (d, 1=10.4 Hz, 2H), 1.57-1.43 (m, 2H). 19F NMR (400 MHz, Dimethysulfoxide-dr) 6-87.27.

### Example 543: 4-((1-(4-(2-(2-Aminopyridin-3-yl)-5-(2-(difluoromethoxy)pyridin-4-yl)-3H-imidazo[4,5-b]pyridin-3-yl)benzyl)piperidin-4-yl)amino)pyrimidine-2-carbonitrile

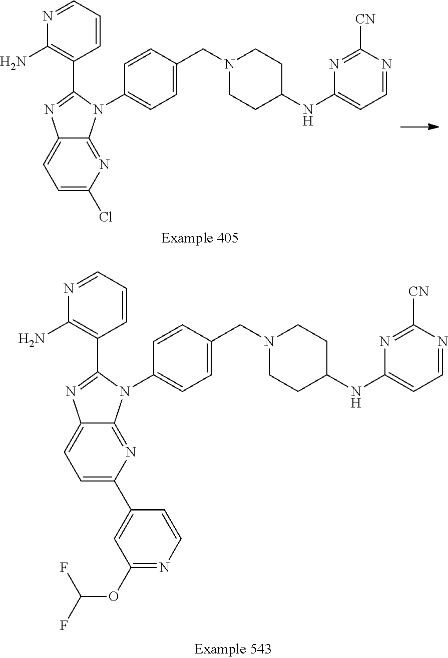 [SMILES: N#Cc1nccc(NC2CCN(Cc3ccc(-n4c(-c5cccnc5N)nc5ccc(Cl)nc54)cc3)CC2)n1 ; N#Cc1nccc(NC2CCN(Cc3ccc(-n4c(-c5cccnc5N)nc5ccc(-c6ccnc(OC(F)F)c6)nc54)cc3)CC2)n1]

A mixture of (2-(difluoromethoxy)pyridin-4-yl)boronic acid (93 mg, 492 μmol), Example 405 (220 mg, 410 μmol), Cs₂CO₃ (400 mg, 1.2 mmol), Pd(dppf)Cl₂ (30 mg, 41 μmol) in H₂O (0.5 mL) and 1,4-dioxane (1 mL) was degassed and purged with N₂ three times. The mixture was stirred at 100° C. for 1 hr under N₂ atmosphere. The reaction mixture was diluted with H₂O (30 mL) and extracted with EtOAc (20 mL×3). The combined organic layers were washed with brine (30 mL), dried over Na₂SO₄, filtered, and concentrated under reduced pressure. After purified by prep-HPLC (column: Phenomenex C18 150×25 mm×10 μm; mobile phase: [water (NH₄HCO₃)-ACN]; gradient: 47%-77% B over 11 min), 4-((1-(4-(2-(2-aminopyridin-3-yl)-5-(2-(difluoromethoxy)pyridin-4-yl)-3H-imidazo[4,5-b]pyridin-3-yl)benzyl)piperidin-4-yl)amino)pyrimidine-2-carbonitrile (Example 543, 86.6 mg, yield: 32%) was obtained as an off white solid. MS: m/z=646.3 [M+H]⁺. ¹H NMR (400 MHz, Dimethysulfoxide-d₆) δ8.42-8.31 (m, 2H), 8.21 (d, J=8.4 Hz, 1H), 8.13-7.99 (m, 3H), 7.96-7.87 (m, 1.3H), 7.73 (s, 0.5H), 7.66 (s, 1H), 7.55 (s, 0.3H), 7.53-7.42 (m, 4H), 7.19 (dd, J=7.6, 1.6 Hz, 1H), 7.11-6.97 (m, 2H), 6.67 (d, J=6.0 Hz, 1H), 6.39 (dd, J=7.6, 4.8 Hz, 1H), 3.91-3.74 (m, 1H), 3.60 (s, 2H), 2.83 (d, J=11.2 Hz, 2H), 2.23-2.11 (m, 2H), 1.95-1.80 (m, 2H), 1.59-1.42 (m, 2H). ¹⁹F NMR (400 MHz, Dimethysulfoxide-d₆) δ −87.12.

### Example 544: 4-((1-(4-(2-(2-Aminopyridin-3-yl)-5-(4-fluoro-2-hydroxyphenyl)-3H-imidazo[4,5-b]pyridin-3-yl)benzyl)piperidin-4-yl)amino)pyrimidine-2-carbonitrile

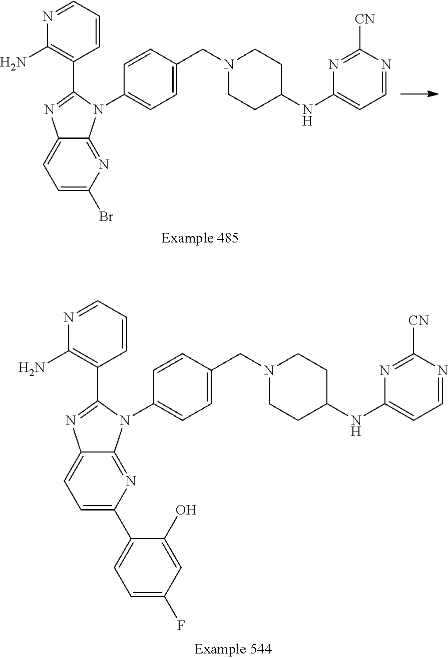 [SMILES: N#Cc1nccc(NC2CCN(Cc3ccc(-n4c(-c5cccnc5N)nc5ccc(Br)nc54)cc3)CC2)n1 ; N#Cc1nccc(NC2CCN(Cc3ccc(-n4c(-c5cccnc5N)nc5ccc(-c6ccc(F)cc6O)nc54)cc3)CC2)n1]

To a solution of Example 485 (200 mg, 344 μmol) and (4-fluoro-2-hydroxy-phenyl)boronic acid (59.0 mg, 378 μmol) in 1,4-dioxane (10 mL) and H₂O (2 mL) were added Cs₂CO₃ (336 mg, 1.03 mmol) and Pd(dppf)Cl₂ (25.2 mg, 34.4 μmol) at 25° C. The mixture was degassed and purged with N₂ three times and stirred at 100° C. for 2 hr. The reaction mixture was quenched with water (20 mL) and extracted with EtOAc (20 mL×3). The combined organic layers were washed with brine (20 mL), dried over Na₂SO₄, filtered and concentrated. The crude was purified by silica gel flash chromatography (MeOH in CH₂Cl₂=0-20%) to give 4-((1-(4-(2-(2-aminopyridin-3-yl)-5-(4-fluoro-2-hydroxyphenyl)-3H-imidazo[4,5-b]pyridin-3-yl)benzyl)piperidin-4-yl)amino)pyrimidine-2-carbonitrile (Example 544, 35.2 mg, yield: 17%) as a yellow solid. MS: m/z=613.2 [M+H]⁺. ¹H NMR (400 MHz, Dimethylsulfoxide-d₆) δ 13.19 (s, 1H), 8.40 (d, J=8.4 Hz, 1H), 8.20-8.05 (m, 4H), 8.02 (dd, J=4.8, 1.6 Hz, 1H), 7.58-7.43 (m, 4H), 7.24 (d, J=6.8 Hz, 1H), 7.08 (s, 2H), 6.77 (td, J=8.4, 2.8 Hz, 1H), 6.71-6.62 (m, 2H), 6.47-6.34 (m, 1H), 3.92-3.69 (m, 1H), 3.61 (s, 2H), 2.93-2.72 (m, 2H), 2.27-2.09 (m, 2H), 1.97-1.83 (m, 2H), 1.57-1.47 (m, 2H). ¹⁹F NMR (400 MHz, Dimethylsulfoxide-d₆) δ −109.982.

### Example 545: 4-((1-(4-(2-(2-Aminopyridin-3-yl)-5-(4-fluoro-3-hydroxyphenyl)-3H-imidazo[4,5-b]pyridin-3-yl)benzyl)piperidin-4-yl)amino)pyrimidine-2-carbonitrile

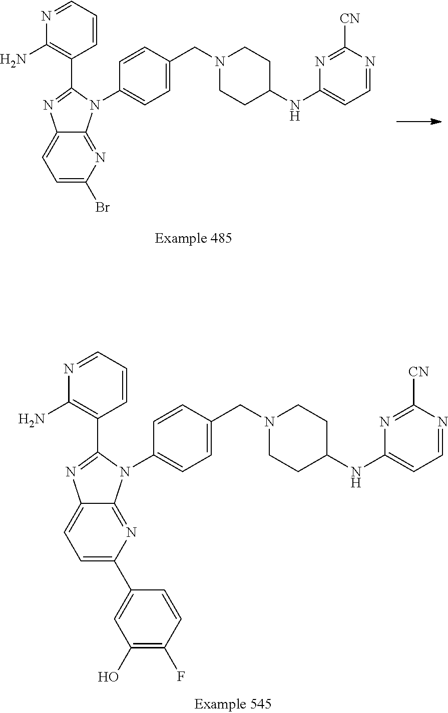 [SMILES: N#Cc1nccc(NC2CCN(Cc3ccc(-n4c(-c5cccnc5N)nc5ccc(Br)nc54)cc3)CC2)n1 ; N#Cc1nccc(NC2CCN(Cc3ccc(-n4c(-c5cccnc5N)nc5ccc(-c6ccc(F)c(O)c6)nc54)cc3)CC2)n1]

To a solution of Example 485 (200 mg, 344 μmol) and (4-fluoro-3-hydroxyphenyl)boronic acid (59.0 mg, 378 μmol) in 1,4-dioxane (10 mL) and H₂O (2 mL) were added Cs₂CO₃ (336 mg, 1.03 mmol) and Pd(dppf)Cl₂ (25.2 mg, 34.4 μmol) at 25° C. The mixture was degassed and purged with N₂ three times and stirred at 100° C. for 2 hr. The reaction mixture was quenched with water (20 mL) and extracted with EtOAc (20 mL×3). The combined organic layers were washed with brine (20 mL), dried over Na₂SO₄, filtered and concentrated. After purified by silica gel flash chromatography (MeOH in CH₂Cl₂=0-20%), 4-((1-(4-(2-(2-aminopyridin-3-yl)-5-(4-fluoro-3-hydroxyphenyl)-3H-imidazo[4,5-b]pyridin-3-yl)benzyl)piperidin-4-yl)amino)pyrimidine-2-carbonitrile (Example 545, 30.0 mg, 14% yield) was obtained as a yellow solid. MS: m/z=613.2 [M+H]⁺. ¹H NMR (400 MHz, Dimethylsulfoxide-d₆) δ 10.00 (s, 1H), 8.24 (d, J=8.4 Hz, 1H), 8.15-8.03 (m, 2H), 8.01-7.96 (m, 1H), 7.88 (d, J=8.4 Hz, 1H), 7.63 (dd, J=9.2, 2.0 Hz, 1H), 7.52-7.40 (m, 5H), 7.26-7.17 (m, 1H), 7.15-7.11 (m, 1H), 7.01 (s, 2H), 6.72-6.64 (m, 1H), 6.42-6.30 (m, 1H), 3.95-3.69 (m, 1H), 3.60 (s, 2H), 2.95-2.78 (m, 2H), 2.23-2.07 (m, 2H), 1.98-1.81 (m, 2H), 1.62-1.53 (m, 2H). ¹⁹F NMR (400 MHz, Dimethylsulfoxide-d₆) δ −136.277.

### Example 546: 4-((1-(4-(2-(2-Aminopyridin-3-yl)-5-(1-methyl-1H-imidazol-5-yl)-3H-imidazo[4,5-b]pyridin-3-yl)benzyl)piperidin-4-yl)amino)pyrimidine-2-carbonitrile

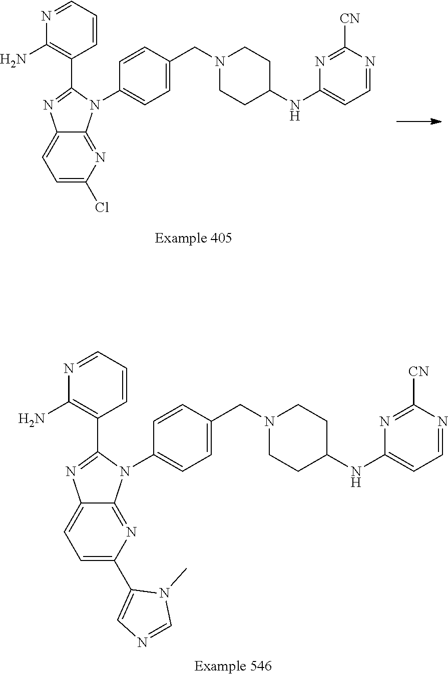 [SMILES: N#Cc1nccc(NC2CCN(Cc3ccc(-n4c(-c5cccnc5N)nc5ccc(Cl)nc54)cc3)CC2)n1 ; Cn1cncc1-c1ccc2nc(-c3cccnc3N)n(-c3ccc(CN4CCC(Nc5ccnc(C#N)n5)CC4)cc3)c2n1]

A mixture of Example 405 (250 mg, 465 μmol), 1-methyl-5-(4,4,5,5-tetramethyl-1,3,2-dioxaborolan-2-yl)-1H-imidazole (194 mg, 931 μmol), Cs₂CO₃ (455 mg, 1.4 mmol), Pd(dppf)Cl₂ (34 mg, 46 μmol) in H₂O (1 mL) and 1,4-dioxane (3 mL) was degassed and purged with N₂ three times. The mixture was stirred at 100° C. for 1 hr under N₂ atmosphere. The reaction mixture was diluted with H₂O (30 mL) and extracted with EtOAc (20 mL×3). The combined organic layers were washed with brine (30 mL), dried over Na₂SO₄, filtered, and concentrated under reduced pressure. After purified by prep-HPLC (column. Phenomenex C18 150×25 mm×10 μm; mobile phase: [water (NH₄HCO₃)-ACN]; gradient: 28%-58% B over 11 min), 4-((1-(4-(2-(2-aminopyridin-3-yl)-5-(1-methyl-1H-imidazol-5-yl)-3H-imidazo[4,5-b]pyridin-3-yl)benzyl)piperidin-4-yl)amino)pyrimidine-2-carbonitrile (Example 546, 78 mg, yield: 29%) was obtained as a yellow solid. MS: m/z=583.3 [M+H]⁺. ¹H NMR (400 MHz, Dimethysulfoxide-d₆) δ 8.21 (d, J=8.4 Hz, 1H), 8.16-7.92 (m, 3H), 7.76 (d, J=8.4 Hz, 1H), 7.67 (s, 1H), 7.55 (s, 1H), 7.49-7.39 (m, 4H), 7.21 (dd, J=7.6, 1.6 Hz, 1H), 7.09 (br s, 2H), 6.67 (d, J=6.0 Hz, 1H), 6.39 (dd, J=7.6, 4.8 Hz, 1H), 3.89-3.77 (m, 1H), 3.75 (s, 3H), 3.58 (s, 2H), 2.86-2.74 (m, 2H), 2.13 (t, J=10.8 Hz, 2H), 1.89 (d, J=10.8 Hz, 2H), 1.56-1.41 (m, 2H).

### Example 547: 4-((1-(4-(2-(2-Aminopyridin-3-yl)-5-(5-methylpyridin-2-yl)-3H-imidazo[4,5-b]pyridin-3-yl)benzyl)piperidin-4-yl)amino)pyrimidine-2-carbonitrile

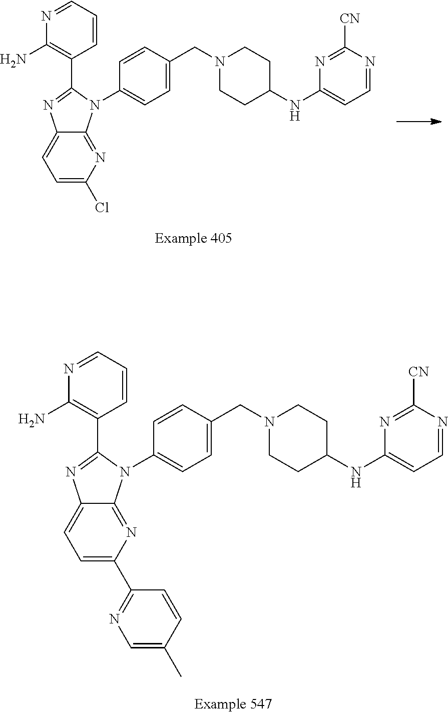 [SMILES: N#Cc1nccc(NC2CCN(Cc3ccc(-n4c(-c5cccnc5N)nc5ccc(Cl)nc54)cc3)CC2)n1 ; Cc1ccc(-c2ccc3nc(-c4cccnc4N)n(-c4ccc(CN5CCC(Nc6ccnc(C#N)n6)CC5)cc4)c3n2)nc1]

A mixture of Example 405 (100 mg, 186 μmol), (5-methylpyridin-2-yl)boronic acid (76.5 mg, 559 μmol), cataCXium A Pd G3 (15.8 mg, 18.6 μmol), CuBr (20 mg, 140 μmol) and Cs₂CO₃ (182 mg, 559 μmol) in 1,4-dioxane (3 mL) was degassed and purged with N₂ three times. The mixture was stirred at 90° C. for 3 hr under N₂ atmosphere. The reaction mixture was poured into H₂O (15 mL) and extracted with EtOAc (15 mL×3). The combined organic phases were dried over anhydrous Na₂SO₄, filtered and concentrated under reduced pressure. After purified by prep-HPLC (column: Waters Xbridge 150×25 mm×5 μm; mobile phase: [water (NH₄HCO₃)-ACN]; gradient: 34%-64% B over 11 min), 4-((1-(4-(2-(2-aminopyridin-3-yl)-5-(5-methylpyridin-2-yl)-3H-imidazo[4,5-h]pyridin-3-yl)benzyl)piperidin-4-yl)amino)pyrimidine-2-carbonitrile (Example 547, 15.5 mg, yield: 14%) was obtained as a yellow solid. MS: m/z=594.3 [M+H]⁺. ¹H NMR (400 MHz, Methanol-d₄) δ 8.46-8.43 (m, 1H), 8.38 (d, J=6.0 Hz, 1H), 8.22 (d, J=8.0 Hz, 1H), 8.17 (d, J=8.0 Hz, 1H), 8.03-7.93 (m, 2H), 7.70-7.63 (m, 1H), 7.57-7.52 (m, 2H), 7.49-7.42 (m, 2H), 7.32 (dd, J=7.6, 1.6 Hz, 1H), 6.63-6.54 (m, 1H), 6.46 (dd, J=7.6, 5.2 Hz, 1H), 4.13-3.79 (m, 1H), 3.66 (s, 2H), 3.00-2.92 (m, 2H), 2.38 (s, 3H), 2.32-2.22 (m, 2H), 2.02-1.97 (m, 2H), 1.68-1.55 (m, 2H).

### Example 548: 4-((1-(4-(2-(2-Aminopyridin-3-yl)-5-(4-cyanopyridin-2-yl)-3H-imidazo[4,5-b]pyridin-3-yl)benzyl)piperidin-4-yl)amino)pyrimidine-2-carbonitrile

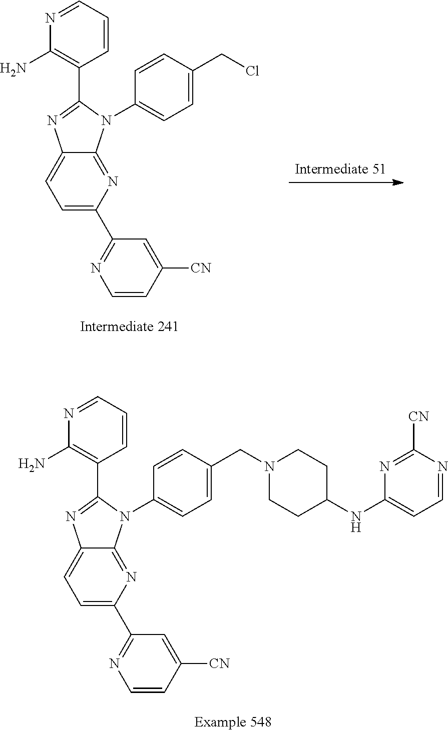 [SMILES: N#Cc1ccnc(-c2ccc3nc(-c4cccnc4N)n(-c4ccc(CCl)cc4)c3n2)c1 ; N#Cc1ccnc(-c2ccc3nc(-c4cccnc4N)n(-c4ccc(CN5CCC(Nc6ccnc(C#N)n6)CC5)cc4)c3n2)c1]

To a solution of Intermediate 241 (67.9 mg, 143 μmol, HCl salt) and Intermediate 51 (40.8 mg, 129 μmol, TFA salt) in DMF (4 mL) were added K₂CO₃ (59.3 mg, 429 μmol) and NaI (4.29 mg, 28.6 μmol). The mixture was stirred at 50° C. for 1 hr. The reaction mixture was quenched with H₂O (10 mL) at 25° C., diluted with CH₂Cl₂ (10 mL), and extracted with CH₂Cl₂ (15 mL×2). The combined organic layers were washed with brine (30 mL×3), dried over Na₂SO₄, filtered, and concentrated under reduced pressure. After purified by prep-TLC (CH₂Cl₂:MeOH=10:1), 4-((1-(4-(2-(2-aminopyridin-3-yl)-5-(4-cyanopyridin-2-yl)-3H-imidazo[4,5-b]pyridin-3-yl)benzyl)piperidin-4-yl)amino)pyrimidine-2-carbonitrile (Example 548, 5.4 mg, yield: 6.2% for three steps) was obtained as a light-yellow solid. MS: m/z=605.5 [M+H]⁺. ¹H NMR (400 MHz, Chloroform-d) δ 8.81 (d, J=4.8 Hz, 1H), 8.57-8.50 (m, 2H), 8.21 (d, J=8.0 Hz, 1H), 8.18-8.11 (m, 1H), 8.09 (d, J=4.8 Hz, 1H), 7.57 (d, J=8.4 Hz, 2H), 7.45 (d, J=4.8 Hz, 1H), 7.40 (d, J=8.4 Hz, 2H), 7.16 (d, J=7.6 Hz, 1H), 6.69 (br s, 2H), 6.43 (d, J=6.0 Hz, 1H), 6.38 (dd, J=8.0, 4.8 Hz, 1H), 4.22-3.79 (m, 1H), 3.69 (s, 2H), 2.99-2.91 (m, 2H), 2.35-2.27 (m, 2H), 2.12-2.06 (m, 2H), 1.38-1.28 (m, 2H).

### Example 549: 4-((1-(4-(2-(2-Aminopyridin-3-yl)-6-bromo-5-(4-fluorophenyl)-3H-imidazo[4,5-b]pyridin-3-yl)benzyl)piperidin-4-yl)amino)pyrimidine-2-carbonitrile

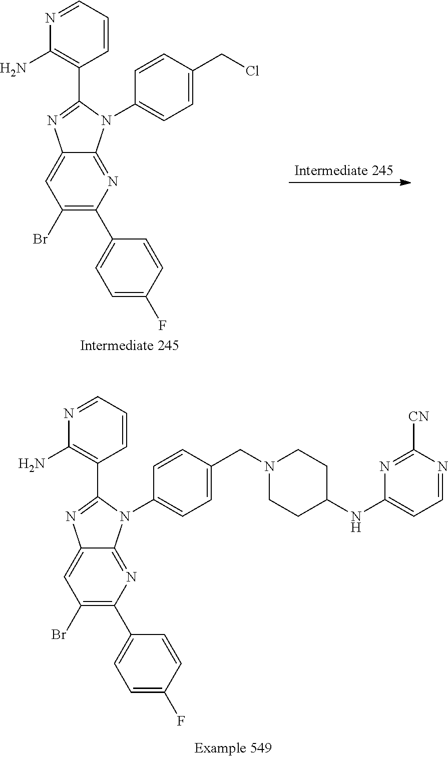 [SMILES: Nc1ncccc1-c1nc2cc(Br)c(-c3ccc(F)cc3)nc2n1-c1ccc(CCl)cc1 ; N#Cc1nccc(NC2CCN(Cc3ccc(-n4c(-c5cccnc5N)nc5cc(Br)c(-c6ccc(F)cc6)nc54)cc3)CC2)n1]

To a solution of Intermediate 245 (266 mg, 488 μmol, HCl salt) and Intermediate 51 (139 mg, 439 μmol, TFA salt) in DMF (5 mL) were added K₂CO₃ (202 mg, 1.46 mmol) and NaI (14.6 mg, 97.6 μmol). The mixture was stirred at 50° C. for 1 hr. The reaction mixture was quenched with H₂O (10 mL) at 25° C., diluted with CH₂Cl₂ (10 mL), and extracted with CH₂Cl₂ (15 mL×2). The combined organic layers were washed with brine (30 mL×3), dried over Na₂SO₄, filtered, and concentrated under reduced pressure. After purified by silica gel flash chromatography (Eluent of 0%˜6% MeOH in CH₂Cl₂), 4-((1-(4-(2-(2-aminopyridin-3-yl)-6-bromo-5-(4-fluorophenyl)-3H-imidazo[4,5-b]pyridin-3-yl)benzyl)piperidin-4-yl)amino)pyrimidine-2-carbonitrile (Example 549, 84.8 mg, yield: 26% for three steps) was obtained as a yellow solid. MS: m/z=675.0, 677.0 [M+H]⁺. ¹H NMR (400 MHz, Dimethylsulfoxide-d₆) δ 8.64 (s, 1H), 8.20-7.93 (m, 3H), 7.66-7.55 (m, 2H), 7.45-7.37 (m, 4H), 7.29-7.27 (m, 2H), 7.15 (dd, J=7.6, 1.6 Hz, 1H), 6.96 (br s, 2H), 6.66 (d, J=6.0 Hz, 1H), 6.38 (dd, J=7.6, 4.8 Hz, 1H), 3.85-3.71 (m, 1H), 3.54 (s, 2H), 2.83-2.72 (m, 2H), 2.18-2.08 (m, 2H), 1.92-1.77 (m, 2H), 1.53-1.38 (m, 2H).

### Example 550: 2-(2-Aminopyridin-3-yl)-3-(4-((4-((2-cyanopyrimidin-4-yl)amino)piperidin-1-yl)methyl)phenyl)-5-methyl-3H-imidazo[4,5-b]pyridine-6- carbonitrile

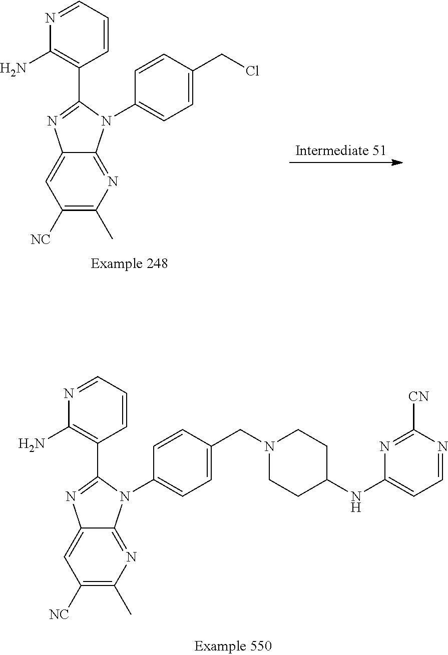 [SMILES: Cc1nc2c(cc1C#N)nc(-c1cccnc1N)n2-c1ccc(CCl)cc1 ; Cc1nc2c(cc1C#N)nc(-c1cccnc1N)n2-c1ccc(CN2CCC(Nc3ccnc(C#N)n3)CC2)cc1]

To a solution of Example 248 (196 mg, 477 μmol, HCl salt) and Intermediate 51 (151 mg, 477 μmol, TFA salt) in DMF (5 mL) were added K₂CO₃ (198 mg, 1.43 mmol) and NaI (14.3 mg, 95.3 μmol). The mixture was stirred at 50° C. for 1 hr. The reaction mixture was quenched with H₂O (10 mL) at 25° C., diluted with CH₂Cl₂ (10 mL), and extracted with CH₂Cl₂ (15 mL×2). The combined organic layers were washed with brine (30 mL×3), dried over Na₂SO₄, filtered, and concentrated under reduced pressure. After purified by silica gel flash chromatography (Eluent of 0˜6% MeOH in CH₂Cl₂), 2-(2-aminopyridin-3-yl)-3-(4-((4-((2-cyanopyrimidin-4-yl)amino)piperidin-1-yl)methyl)phenyl)-5-methyl-3H-imidazo[4,5-b]pyridine-6-carbonitrile (Example 550, 120 mg, yield: 46% for three steps) was obtained as a yellow solid. MS: m/z=542.2 [M+H]⁺. ¹H NMR (400 MHz, Dimethylsulfoxide-d₆) δ 8.72 (s, 1H), 8.13-8.03 (m, 2H), 8.00 (dd, J=4.4, 1.6 Hz, 1H), 7.51-7.43 (m, 2H), 7.42-7.36 (m, 2H), 7.16 (dd, J=7.6, 1.6 Hz, 1H), 6.93 (br s, 2H), 6.67 (d, J=6.0 Hz, 1H), 6.38 (dd, J=7.6, 4.8 Hz, 1H), 3.87-3.75 (m, 1H), 3.57 (s, 2H), 2.85-2.77 (m, 2H), 2.70 (s, 3H), 2.22-2.10 (m, 2H), 1.92-1.83 (m, 2H), 1.56-1.43 (m, 2H).

### Example 551: 4-((1-(4-(2-(2-aminopyridin-3-yl)-5-(3-fluoropyridin-4-yl)-3H-imidazo[4,5-b]pyridin-3-yl)benzyl)piperidin-4-yl)amino)pyrimidine-2-carbonitrile

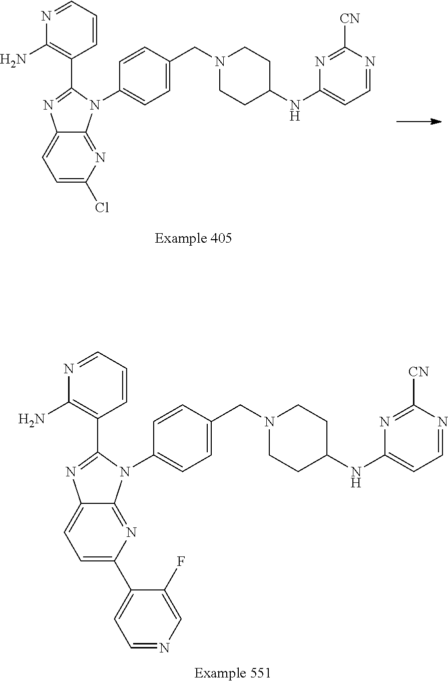 [SMILES: N#Cc1nccc(NC2CCN(Cc3ccc(-n4c(-c5cccnc5N)nc5ccc(Cl)nc54)cc3)CC2)n1 ; N#Cc1nccc(NC2CCN(Cc3ccc(-n4c(-c5cccnc5N)nc5ccc(-c6ccncc6F)nc54)cc3)CC2)n1]

A mixture of Example 405 (100 mg, 186 μmol), (3-fluoropyridin-4-yl)boronic acid (26.2 mg, 186 μmol), Na₂CO₃ (59.2 mg, 559 μmol), Pd(dppf)Cl₂ (13.6 mg, 18.6 μmol) in H₂O (0.5 mL) and 1,4-dioxane (2 mL) was degassed and purged with N₂ three times. The mixture was stirred at 100° C. for 16 hr under N₂ atmosphere. The reaction mixture was poured into H₂O (15 mL) and extracted with EtOAc (15 mL×3). The combined organic layers were dried over anhydrous Na₂SO₄, filtered and concentrated under reduced pressure. After purified by prep-HPLC (column: Phenomenex C18 150×25 mm×10 μm; mobile phase: [water (NH₄HCO₃)-ACN]; gradient: 38%-68% B over 11 min), 4-((1-(4-(2-(2-aminopyridin-3-yl)-5-(3-fluoropyridin-4-yl)-3H-imidazo[4,5-b]pyridin-3-yl)benzyl)piperidin-4-yl)amino)pyrimidine-2-carbonitrile (Example 551, 19.2 mg, yield: 16%) was obtained as a pink lyophilized powder. MS: m/z=598.2 [M+H]⁺. ¹H NMR (400 MHz, Dimethylsulfoxide-d₆) δ 8.69 (d, J=2.8 Hz, 1H), 8.51 (d, J=4.8 Hz, 1H), 8.36 (d, J=8.0 Hz, 1H), 8.14-7.97 (m, 3H), 7.97-7.91 (m, 1H), 7.85 (dd, J=6.8, 5.2 Hz 1H), 7.48-7.43 (m, 4H), 7.18 (dd, J=7.6, 1.6 Hz 1H), 6.99 (s, 2H), 6.66 (d, J=6.4 Hz, 1H), 6.38 (dd, J=7.6, 4.8 Hz 1H), 3.90-3.71 (m, 1H), 3.57 (s, 2H), 2.87-2.74 (m, 2H), 2.21-2.05 (m, 2H), 1.94-1.81 (m, 2H), 1.58-1.37 (m, 2H). ¹⁹F NMR (400 MHz, Dimethylsulfoxide-d₆) δ −131.931.

### Example 552: 4-((1-(4-(2-(2-Aminopyridin-3-yl)-5-(5-methoxypyridin-2-yl)-3H-imidazo[4,5-b]pyridin-3-yl)benzyl)piperidin-4-yl)amino)pyrimidine-2-carbonitrile

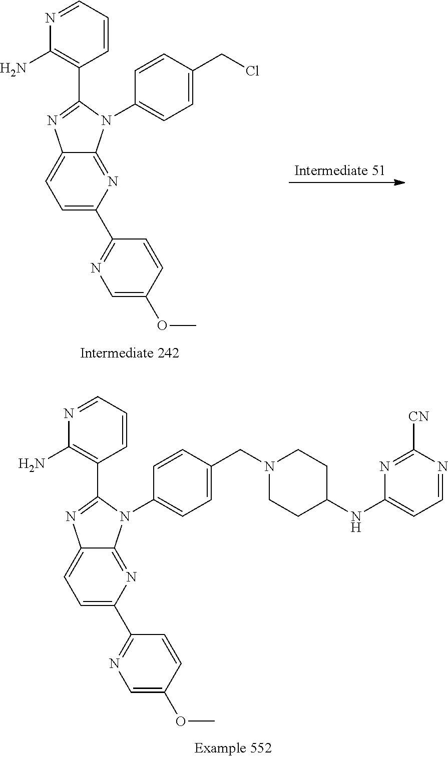 [SMILES: COc1ccc(-c2ccc3nc(-c4cccnc4N)n(-c4ccc(CCl)cc4)c3n2)nc1 ; COc1ccc(-c2ccc3nc(-c4cccnc4N)n(-c4ccc(CN5CCC(Nc6ccnc(C#N)n6)CC5)cc4)c3n2)nc1]

To a solution of Intermediate 242 (150 mg, 339 μmol, HCl salt) and Intermediate 51 (103 mg, 325 μmol, TFA salt) in DMF (5 mL) were added K₂CO₃ (140 mg, 1.02 mmol) and NaI (10.2 mg, 67.7 μmol). The mixture was stirred at 50° C. for 0.5 hr. The reaction mixture was poured into H₂O (50 mL) and extracted with CH₂Cl₂ (15 mL×3). The combined organic layers were dried over anhydrous Na₂SO₄, filtered, and concentrated under reduced pressure. After purified by silica gel flash chromatography (Eluent of 0%˜6% MeOH in CH₂C2), 4-((1-(4-(2-(2-aminopyridin-3-yl)-5-(5-methoxypyridin-2-yl)-3H-imidazo[4,5-b]pyridin-3-yl)benzyl)piperidin-4-yl)amino)pyrimidine-2-carbonitrile (Example 552, 50.2 mg, yield: 24%) was obtained as a light-yellow solid. MS: m/z=610.2 [M+H]⁺. ¹H NMR (400 MHz, Dimethylsulfoxide-d₆) δ 8.39-8.33 (m, 2H), 8.26 (d, J=8.4 Hz, 1H), 8.13 (d, J=8.8 Hz, 1H), 8.09-8.03 (m, 2H), 8.00 (dd, J=4.8, 1.6 Hz, 1H), 7.51-7.44 (m, 5H), 7.15 (dd, J=7.6, 1.6 Hz, 1H), 7.02 (s, 2H), 6.68 (d, J=6.0 Hz, 1H), 6.38 (dd, J=7.6, 4.8 Hz, 1H), 3.87 (s, 3H), 3.85-3.77 (m, 1H), 3.60 (s, 2H), 2.88-2.79 (m, 2H), 2.22-2.12 (m, 2H), 1.96-1.84 (m, 2H), 1.56-1.45 (m, 2H).

### Example 553: 2-(2-Aminopyridin-3-yl)-1-(4-((4-((2-cyanopyrimidin-4-yl)amino)piperidin-1-yl)methyl)phenyl)-1H-imidazo[4,5-c]pyridine-6-carbonitrile

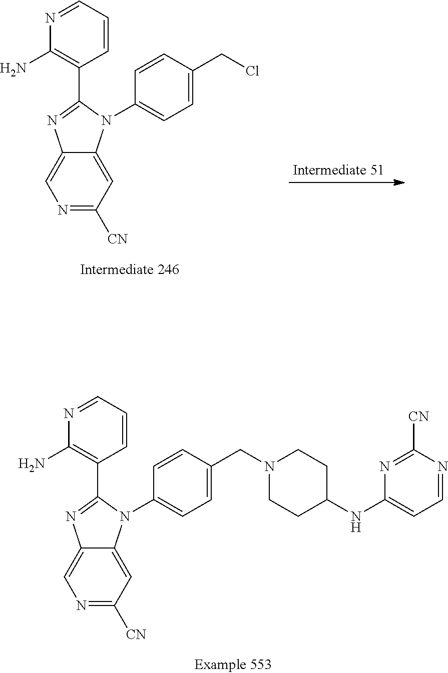 [SMILES: N#Cc1cc2c(cn1)nc(-c1cccnc1N)n2-c1ccc(CCl)cc1 ; N#Cc1cc2c(cn1)nc(-c1cccnc1N)n2-c1ccc(CN2CCC(Nc3ccnc(C#N)n3)CC2)cc1]

To a solution of Intermediate 246 (160 mg, 443 μmol, HCl salt) and Intermediate 51 (141 mg, 443 μmol, TFA salt) in DMF (5 mL) were added K₂CO₃ (184 mg, 1.33 mmol) and NaI (13.3 mg, 88.7 μmol). The mixture was stirred at 50° C. for 0.5 hr. The reaction mixture was quenched with H₂O (50 mL) at 25° C., diluted with CH₂Cl₂ (50 mL), and extracted with CH₂Cl₂ (50 mL×2). The combined organic layers were washed with brine (30 mL×3), dried over Na₂SO₄, filtered, and concentrated under reduced pressure. After purified by silica gel flash chromatography (Eluent of 0%˜6% MeOH in CH₂Cl₂), 2-(2-aminopyridin-3-yl)-1-(4-((4-((2-cyanopyrimidin-4-yl)amino)piperidin-1-yl)methyl)phenyl)-1H-imidazo[4,5-c]pyridine-6-carbonitrile (Example 553, 64.0 mg, yield: 27%) was obtained as a yellow solid. MS: m/z=528.2 [M+H]⁺. ¹H NMR (400 MHz, Dimethylsulfoxide-d₆) δ 9.22 (d, J=0.8 Hz, 1H), 8.12-8.04 (m, 3H), 8.03-8.02 (m, 1H), 7.51-7.48 (m, 2H), 7.45-7.43 (m, 2H), 7.22 (dd, J=7.6, 1.6 Hz, 1H), 6.88 (s, 2H), 6.67 (d, J=6.0 Hz, 1H), 6.41 (dd, J=7.6, 4.8 Hz, 1H), 3.88-3.73 (m, 1H), 3.58 (s, 2H), 2.82 (d, J=11.6 Hz, 2H), 2.18-2.12 (m, 2H), 1.89 (d, J=10.4 Hz, 2H), 1.54-1.47 (m, 2H).

### Example 554: 4-((1-(4-(2-(2-Aminopyridin-3-yl)-5-(4-methyloxazol-5-yl)-3H-imidazo[4,5-b]pyridin-3-yl)benzyl)piperidin-4-yl)amino)pyrimidine-2-carbonitrile

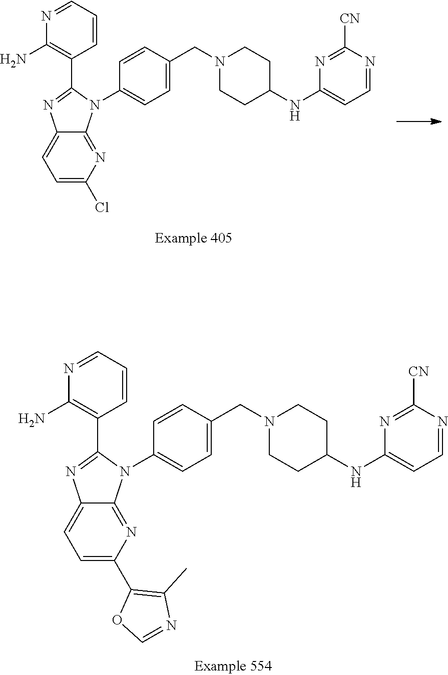 [SMILES: N#Cc1nccc(NC2CCN(Cc3ccc(-n4c(-c5cccnc5N)nc5ccc(Cl)nc54)cc3)CC2)n1 ; Cc1ncoc1-c1ccc2nc(-c3cccnc3N)n(-c3ccc(CN4CCC(Nc5ccnc(C#N)n5)CC4)cc3)c2n1]

A mixture of Example 405 (300 mg, 559 μmol), 4-methyl-5-(4,4,5,5-tetramethyl-1,3,2-dioxaborolan-2-yl)oxazole (128 mg, 615 μmol), Cs₂CO₃ (546 mg, 1.68 mmol), Pd(dppf)Cl₂ (40.9 mg, 55.9 μmol) in H₂O (0.2 mL) and 1,4-dioxane (1 mL) was degassed and purged with N₂ three times. The mixture was stirred at 100° C. for 16 hr under N₂ atmosphere. The reaction mixture was poured into H₂O (50 mL) and extracted with EtOAc (50 mL×3). The combined organic layers were dried over anhydrous Na₂SO₄, filtered, and concentrated under reduced pressure. After purified by prep-HPLC (column: Waters xbridge BEH C18 150×25 mm×5 μm; mobile phase: [water (NH₄HCO₃)-ACN]; gradient: 33%-63% B over 10 min), 4-((1-(4-(2-(2-aminopyridin-3-yl)-5-(4-methyloxazol-5-yl)-3H-imidazo[4,5-b]pyridin-3-yl)benzyl)piperidin-4-yl)amino)pyrimidine-2-carbonitrile (Example 554, 66.8 mg, yield: 19%) was obtained as a light-yellow lyophilized powder. MS: m/z=584.2 [M+H]⁺. ¹H NMR (400 MHz, Dimethylsulfoxide-d₆) δ 8.38 (s, 1H), 8.29 (d, J=8.4 Hz, 1H), 8.11-8.01 (m, 3H), 7.71 (d, J=8.4 Hz, 1H), 7.50-7.47 (m, 2H), 7.46-7.43 (m, 2H), 7.27-7.24 (m, 1H), 7.10 (s, 2H), 6.68 (d, J=6.0 Hz, 1H), 6.41 (dd, J=7.6, 4.8 Hz, 1H), 3.90-3.73 (m, 1H), 3.59 (s, 2H), 2.80 (d, J=10.8 Hz, 2H), 2.35 (s, 3H), 2.17-2.10 (m, 2H), 1.93-1.85 (m, 2H), 1.54-1.46 (m, 2H).

### Example 555: 4-((1-(4-(2-(2-Aminopyridin-3-yl)-5-(1-cyclopropyl-1H-pyrazol-4-yl)-3H-imidazo[4,5-b]pyridin-3-yl)benzyl)piperidin-4-yl)amino)pyrimidine-2-carbonitrile

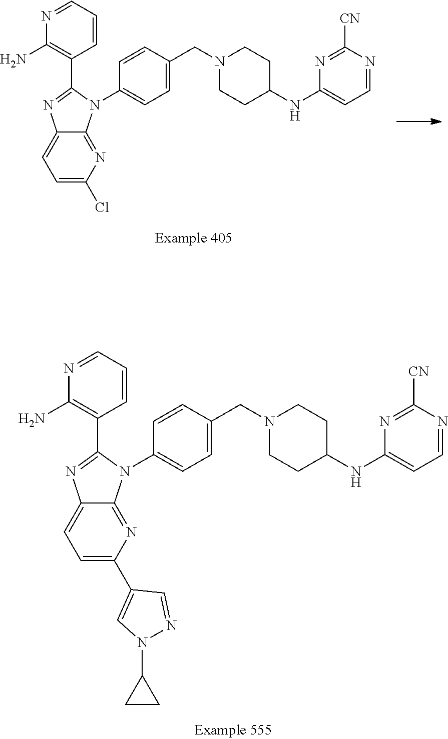 [SMILES: N#Cc1nccc(NC2CCN(Cc3ccc(-n4c(-c5cccnc5N)nc5ccc(Cl)nc54)cc3)CC2)n1 ; N#Cc1nccc(NC2CCN(Cc3ccc(-n4c(-c5cccnc5N)nc5ccc(-c6cnn(C7CC7)c6)nc54)cc3)CC2)n1]

A mixture of Example 405 (300 mg, 559 μmol), (1-cyclopropyl-1H-pyrazol-4-yl)boronic acid (93.4 mg, 615 μmol), Cs₂CO₃ (546 mg, 1.68 mmol), Pd(dppf)Cl₂ (40.9 mg, 55.9 μmol) in H₂O (0.2 mL) and 1,4-dioxane (1 mL) was degassed and purged with N₂ three times. The mixture was stirred at 100° C. for 16 hr under N₂ atmosphere. The reaction mixture was poured into H₂O (50 mL) and extracted with EtOAc (50 mL×3). The combined organic layers were dried over anhydrous Na₂SO₄, filtered, and concentrated under reduced pressure. After purified by prep-HPLC (column: Waters xbridge BEH C18 150×25 mm×5 μm; mobile phase: [water (NH₄HCO₃)-ACN]; gradient: 31%-61% B over 10 min), 4-((1-(4-(2-(2-aminopyridin-3-yl)-5-(1-cyclopropyl-1H-pyrazol-4-yl)-3H-imidazo[4,5-b]pyridin-3-yl)benzyl)piperidin-4-yl)amino)pyrimidine-2-carbonitrile (Example 555, 77.6 mg, yield: 22%) was obtained as a light-yellow lyophilized powder. MS: m/z=609.3 [M+H]⁺. ¹H NMR (400 MHz, Dimethylsulfoxide-d₆) δ 8.25 (s, 1H), 8.15 (d, J=8.4 Hz, 1H), 8.11-8.03 (m, 2H), 7.98 (dd, J=4.8, 1.6 Hz, 1H), 7.89 (s, 1H), 7.68 (d, J=8.4 Hz, 1H), 7.49-7.45 (m, 2H), 7.42-7.38 (m, 2H), 7.10 (d, J=6.4 Hz, 1H), 7.00 (s, 2H), 6.68 (d, J=6.0 Hz, 1H), 6.36 (dd, J=7.6, 4.8 Hz, 1H), 3.88-3.71 (m, 2H), 3.59 (s, 2H), 2.83 (d, J=11.2 Hz, 2H), 2.20-2.11 (m, 2H), 1.94-1.85 (m, 2H), 1.55-1.45 (m, 2H), 1.09-1.04 (m, 2H), 1.00-0.93 (m, 2H).

### Example 556: 2-(2-Aminopyridin-3-yl)-1-(4-((4-((2-cyanopyrimidin-4-yl)amino)piperidin-1-yl)methyl)phenyl)-1H-imidazo[4,5-b]pyrazine-5,6-dicarbonitrile

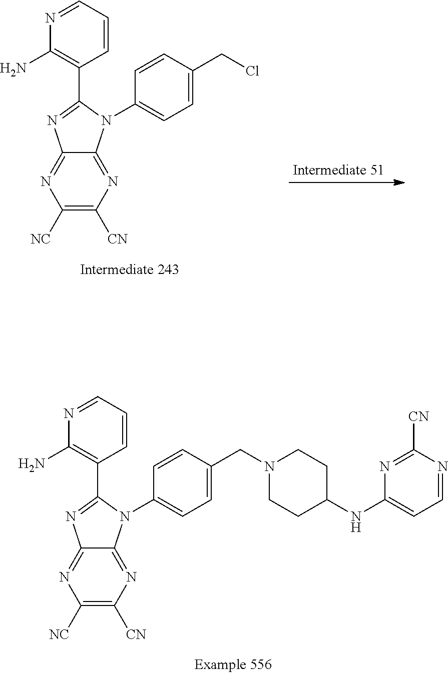 [SMILES: N#Cc1nc2nc(-c3cccnc3N)n(-c3ccc(CCl)cc3)c2nc1C#N ; N#Cc1nccc(NC2CCN(Cc3ccc(-n4c(-c5cccnc5N)nc5nc(C#N)c(C#N)nc54)cc3)CC2)n1]

To a solution of Intermediate 243 (379 mg, 894 μmol, HCl salt) and Intermediate 51 (214 mg, 894 μmol, TFA) in DMF (5 mL) were added K₂CO₃ (371 mg, 2.68 mmol) and NaI (134 mg, 894 μmol). The mixture was degassed and purged with N₂ three times and stirred at 50° C. for 1 hr under N₂. The reaction mixture was diluted with CH₂Cl₂ (50 mL) at 25° C., washed with brine (30 mL×5), dried over Na₂SO₄, filtered and concentrated under reduced pressure. After purified by silica gel flash chromatography (MeOH in CH₂Cl₂=1% to 10%), 2-(2-aminopyridin-3-yl)-1-(4-((4-((2-cyanopyrimidin-4-yl)amino)piperidin-1-yl)methyl)phenyl)-1H-imidazo[4,5-b]pyrazine-5,6-dicarbonitrile (Example 556, 118 mg, yield: 23%) was obtained as a yellow solid. MS: m/z=554.2 [M+H]⁺. ¹H NMR (400 MHz, Dimethylsulfoxide-d₆) δ 8.13 (dd, J=4.4, 1.6 Hz, 1H), 8.09-8.05 (m, 2H), 7.56-7.51 (m, 2H), 7.48-7.44 (m, 2H), 7.29 (dd, J=8.0, 1.6 Hz, 1H), 7.20 (br s, 2H), 6.67 (d, J=6.0 Hz, 1H), 6.46 (dd, J=8.0, 4.8 Hz, 1H), 3.87-3.76 (m, 1H), 3.59 (s, 2H), 2.86-2.76 (m, 2H), 2.21-2.10 (m, 2H), 1.94-1.83 (m, 2H), 1.57-1.41 (m, 2H).

### Example 557: 2-(2-Aminopyridin-3-yl)-3-(4-((4-((2-cyanopyrimidin-4-yl)amino)piperidin-1-yl)methyl)phenyl)-5-(4-fluorophenyl)-3H-imidazo[4,5-b]pyridine-6-carbonitrile

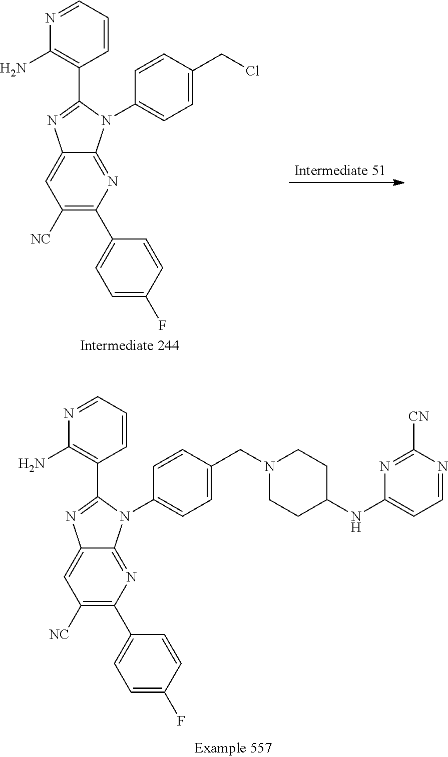 [SMILES: N#Cc1cc2nc(-c3cccnc3N)n(-c3ccc(CCl)cc3)c2nc1-c1ccc(F)cc1 ; N#Cc1nccc(NC2CCN(Cc3ccc(-n4c(-c5cccnc5N)nc5cc(C#N)c(-c6ccc(F)cc6)nc54)cc3)CC2)n1]

To a solution of Intermediate 244 (304 mg, 619 μmol, HCl salt) and Intermediate 51 (196 mg, 619 μmol, TFA salt) in DMF (5 mL) were added K₂CO₃ (257 mg, 1.86 mmol) and NaI (18.6 mg, 124 μmol). The mixture was stirred at 50° C. for 1 hr. The reaction mixture was quenched with H₂O (10 mL) at 25° C., diluted with CH₂Cl₂ (10 mL), and extracted with CH₂Cl₂ (15 mL×2). The combined organic layers were washed with brine (30 mL×3), dried over Na₂SO₄, filtered, and concentrated under reduced pressure. After triturated with CH₂Cl₂ (20 mL) at 25° C. for 10 min, 2-(2-aminopyridin-3-yl)-3-(4-((4-((2-cyanopyrimidin-4-yl)amino)piperidin-1-yl)methyl)phenyl)-5-(4-fluorophenyl)-3H-imidazo[4,5-b]pyridine-6-carbonitrile (Example 557, 133 mg, yield: 34% for three steps) was obtained as a yellow solid. MS: m/z=622.2 [M+H]⁺. ¹H NMR (400 MHz, Dimethylsulfoxide-d₆) δ 8.90 (s, 1H), 8.13-7.99 (m, 3H), 7.83 (dd, J=8.4, 5.2 Hz, 2H), 7.48-7.42 (m, 4H), 7.38-7.36 (m, 2H), 7.19 (dd, J=7.6, 1.2 Hz, 1H), 6.94 (br s, 2H), 6.69 (d, J=6.0 Hz, 1H), 6.40 (dd, J=7.6, 4.8 Hz, 1H), 3.90-3.70 (m, 1H), 3.56 (s, 2H), 2.84-2.76 (m, 2H), 2.19-2.06 (m, 2H), 1.92-1.77 (m, 2H), 1.56-1.41 (m, 2H). ¹⁹F NMR (400 MHz, Dimethylsulfoxide-d₆) δ −111.61.

### Example 558: 4-((1-(4-(2-(2-Aminopyridin-3-yl)-5-(2-fluoropyridin-3-yl)-3H-imidazo[4,5-b]pyridin-3-yl)benzyl)piperidin-4-yl)amino)pyrimidine-2-carbonitrile

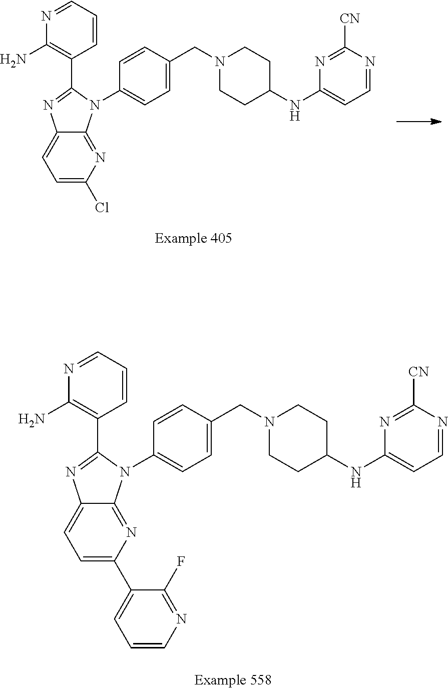 [SMILES: N#Cc1nccc(NC2CCN(Cc3ccc(-n4c(-c5cccnc5N)nc5ccc(Cl)nc54)cc3)CC2)n1 ; N#Cc1nccc(NC2CCN(Cc3ccc(-n4c(-c5cccnc5N)nc5ccc(-c6cccnc6F)nc54)cc3)CC2)n1]

A mixture of Example 405 (200 mg, 372 μmol), (2-fluoropyridin-3-yl)boronic acid (78.7 mg, 559 μmol), Cs₂CO₃ (364 mg, 1.12 mmol), Pd(dppf)Cl₂ (27.2 mg, 37.2 μmol) in H₂O (1 mL) and 1,4-dioxane (4 mL) was degassed and purged with N₂ three times. The mixture was stirred at 100° C. for 16 hr under N₂ atmosphere. The reaction mixture was poured into H₂O (15 mL) and extracted with EtOAc (15 mL×3). The combined organic layers were dried over anhydrous Na₂SO₄, filtered, and concentrated under reduced pressure. After purified by silica gel flash chromatography (Eluent of 0%˜7% MeOH in CH₂Cl₂), 4-((1-(4-(2-(2-aminopyridin-3-yl)-5-(2-fluoropyridin-3-yl)-3H-imidazo[4,5-b]pyridin-3-yl)benzyl)piperidin-4-yl)amino)pyrimidine-2-carbonitrile (Example 558, 52.9 mg, yield: 24%) was obtained as a light brown solid. MS: m/z=598.1 [M+H]⁺. ¹H NMR (400 MHz, Dimethylsulfoxide-d₆) δ 8.43-8.24 (m, 3H), 8.16-7.94 (m, 3H), 7.88 (dd, J=8.0, 4.4 Hz, 1H), 7.53-7.40 (m, 5H), 7.17 (dd, J=7.6, 1.6 Hz, 1H), 6.99 (s, 2H), 6.73-6.61 (m, 1H), 6.39 (dd, J=7.6, 4.8 Hz, 1H), 3.90-3.75 (m, 1H), 3.58 (s, 2H), 2.89-2.77 (m, 2H), 2.22-2.09 (m, 2H), 1.97-1.79 (m, 2H), 1.56-1.34 (m, 2H). ¹⁹F NMR (400 MHz, Dimethylsulfoxide-d₆) δ −70.367.

### Example 559: 4-((1-(4-(2-(2-Aminopyridin-3-yl)-5-(1-methyl-1H-1,2,4-triazol-3-yl)-3H-imidazo[4,5-b]pyridin-3-yl)benzyl)piperidin-4-yl)amino)pyrimidine-2-carbonitrile

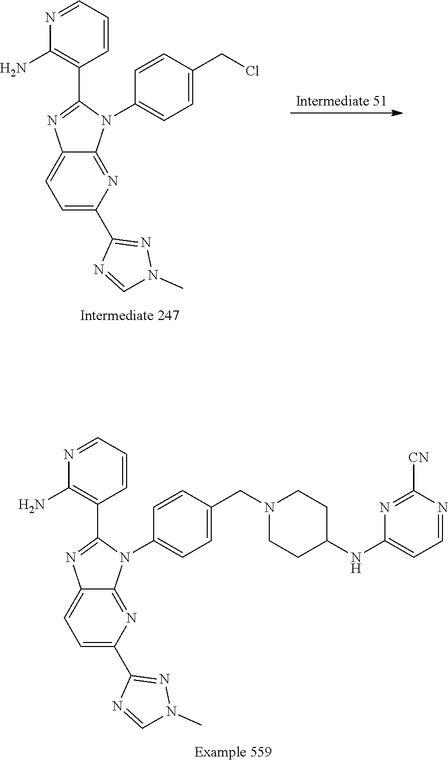 [SMILES: Cn1cnc(-c2ccc3nc(-c4cccnc4N)n(-c4ccc(CCl)cc4)c3n2)n1 ; Cn1cnc(-c2ccc3nc(-c4cccnc4N)n(-c4ccc(CN5CCC(Nc6ccnc(C#N)n6)CC5)cc4)c3n2)n1]

To a solution of Intermediate 247 (156 mg, 374 μmol) and Intermediate 51 (119 mg, 374 μmol, TFA salt) in DMF (5 mL) were added K₂CO₃ (259 mg, 1.87 mmol) and NaI (5.61 mg, 37.4 μmol). The mixture was stirred at 50° C. for 1 hr. The reaction mixture was quenched with H₂O (50 mL) at 25° C. and extracted with CH₂Cl₂ (50 mL×2). The combined organic layers were washed with brine (50 mL×3), dried over Na₂SO₄, filtered, and concentrated under reduced pressure. After purified by silica gel flash chromatography (Eluent of 1%˜8% MeOH in CH₂Cl₂), 4-((1-(4-(2-(2-aminopyridin-3-yl)-5-(1-methyl-1H-1,2,4-triazol-3-yl)-3H-imidazo[4,5-b]pyridin-3-yl)benzyl)piperidin-4-yl)amino)pyrimidine-2-carbonitrile (Example 559, 82.9 mg, yield: 37% for two steps) was obtained as a brown solid. MS: m/z=584.2 [M+H]⁺. ¹H NMR (400 MHz, Dimethylsulfoxide-d₆) δ 8.52 (s, 1H), 8.26 (d, J=8.4 Hz, 1H), 8.13-8.03 (m, 3H), 7.98 (dd, J=4.8, 1.6 Hz, 1H), 7.51-7.41 (m, 4H), 7.19-7.11 (m, 1H), 6.97 (s, 2H), 6.68 (d, J=5.2 Hz, 1H), 6.40-6.32 (m, 1H), 3.92 (s, 3H), 3.88-3.75 (m, 1H), 3.59 (s, 2H), 2.91-2.80 (m, 2H), 2.22-2.12 (m, 2H), 1.93-1.83 (m, 2H), 1.55-1.46 (m, 2H).

### Example 560: 4-((1-(4-(2-(2-Aminopyridin-3-yl)-5-(5-ethoxypyridin-2-yl)-3H-imidazo[4,5-b]pyridin-3-yl)benzyl)piperidin-4-yl)amino)pyrimidine-2-carbonitrile

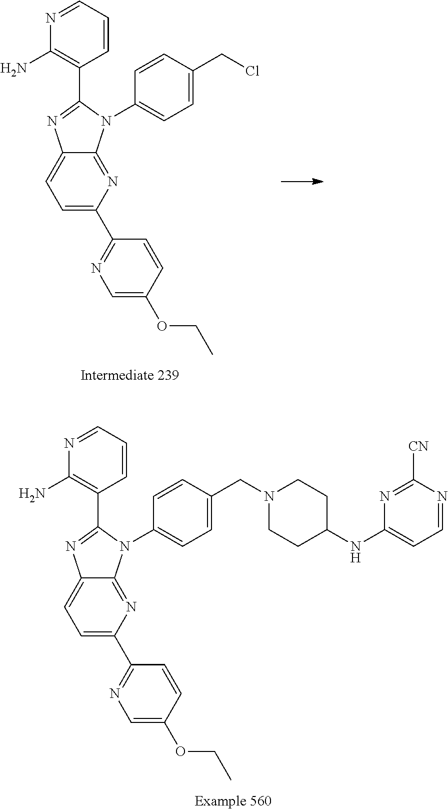 [SMILES: CCOc1ccc(-c2ccc3nc(-c4cccnc4N)n(-c4ccc(CCl)cc4)c3n2)nc1 ; CCOc1ccc(-c2ccc3nc(-c4cccnc4N)n(-c4ccc(CN5CCC(Nc6ccnc(C#N)n6)CC5)cc4)c3n2)nc1]

To a solution of Intermediate 239 (225 mg, 456 μmol, HCl salt) and Intermediate 51 (145 mg, 456 μmol, TFA) in DMF (4 mL) were added K₂CO₃ (315 mg, 2.28 mmol) and NaI (6.84 mg, 45.6 μmol). The mixture was stirred at 50° C. for 1 hr. The reaction mixture was quenched with H₂O (5 mL) at 25° C. and extracted with CH₂Cl₂ (15 mL×2). The combined organic layers were washed with brine (50 mL×3), dried over Na₂SO₄, filtered, and concentrated under reduced pressure. After purified by silica gel flash chromatography (Eluent of 1%˜6% MeOH in CH₂Cl₂), 4-((1-(4-(2-(2-aminopyridin-3-yl)-5-(5-ethoxypyridin-2-yl)-3H-imidazo[4,5-b]pyridin-3-yl)benzyl)piperidin-4-yl)amino)pyrimidine-2-carbonitrile (Example 560, 176 mg, yield: 61% for two steps) was obtained as a yellow solid. MS: m/z=624.3 [M+H]⁺. ¹H NMR (400 MHz, Dimethylsulfoxide-d₆) δ 8.39-8.32 (m, 2H), 8.26 (d, J=8.4 Hz, 1H), 8.15-8.03 (m, 3H), 8.00 (dd, J=5.2, 2.0 Hz, 1H), 7.52-7.43 (m, 5H), 7.16 (dd, J=7.6, 1.6 Hz, 1H), 7.02 (br s, 2H), 6.68 (d, J=6.0 Hz, 1H), 6.38 (dd, J=7.6, 4.8 Hz, 1H), 4.15 (q, J=6.8 Hz, 2H), 3.90-3.75 (m, 1H), 3.60 (s, 2H), 2.90-2.80 (m, 2H), 2.22-2.12 (m, 2H), 1.97-1.83 (m, 2H), 1.57-1.45 (m, 2H), 1.36 (t, J=7.2 Hz, 3H).

### Example 561: 4-((1-(4-(2-(2-Aminopyridin-3-yl)-5-(2-fluoropyridin-4-yl)-3H-imidazo[4,5-b]pyridin-3-yl)benzyl)piperidin-4-yl)amino)pyrimidine-2-carbonitrile

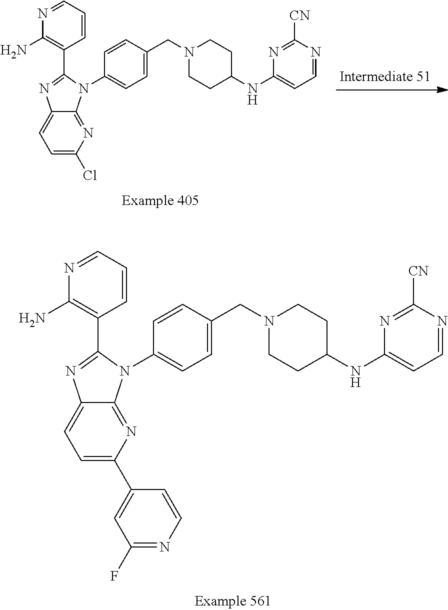 [SMILES: N#Cc1nccc(NC2CCN(Cc3ccc(-n4c(-c5cccnc5N)nc5ccc(Cl)nc54)cc3)CC2)n1 ; N#Cc1nccc(NC2CCN(Cc3ccc(-n4c(-c5cccnc5N)nc5ccc(-c6ccnc(F)c6)nc54)cc3)CC2)n1]

To a mixture of Example 405 (300 mg, 559 μmol) and (2-fluoropyridin-4-yl)boronic acid (86.6 mg, 615 μmol) in 1,4-dioxane (2 mL) and H₂O (0.5 mL) were added Cs₂CO₃ (546 mg, 1.68 mmol) and Pd(dppf)Cl₂ (40.9 mg, 55.9 μmol). The mixture was stirred at 100° C. for 2 hr under N₂. The mixture was diluted with H₂O (50 mL) and extracted with EtOAc (100 mL×2). The combined organic layers were washed with brine (100 mL), dried over anhydrous Na₂SO₄, filtered and concentrated. After purified by prep-HPLC (column: Phenomenex C18 80×30 mm×5 μm; mobile phase. [water (NH₃H₂O+NH₄HCO₃)-ACN]; gradient: 47%-77% B over 7 min), 4-((1-(4-(2-(2-aminopyridin-3-yl)-5-(2-fluoropyridin-4-yl)-3H-imidazo[4,5-b]pyridin-3-yl)benzyl)piperidin-4-yl)amino)pyrimidine-2-carbonitrile (Example 561, 127.9 mg, yield: 38%) was obtained as a yellow solid. MS: m/z=598.3 [M+H]⁺. ¹H NMR (400 MHz, Dimethylsulfoxide-d₆) δ 8.44-8.28 (m, 2H), 8.26-8.16 (m, 1H), 8.12-7.93 (m, 4H), 7.75 (s, 1H), 7.52-7.45 (m, 4H) 7.24-7.13 (m, 1H), 7.04 (br s, 2H), 6.72-6.60 (m, 1H), 6.45-6.33 (m, 1H), 3.88-3.75 (m, 1H), 3.60 (s, 2H), 2.89-2.77 (m, 2H), 2.26-2.08 (m, 2H), 1.96-1.81 (m, 2H), 1.59-1.43 (m, 2H). ¹⁹F NMR (400 MHz, Dimethylsulfoxide-d₆) δ −68.690.

### Example 562: 4-((1-(4-(2-(3-Aminopyrazin-2-yl)-5-(2-methyl-2H-1,2,3-triazol-4-yl)-3H-imidazo[4,5-b]pyridin-3-yl)benzyl)piperidin-4-yl)amino)pyrimidine-2-carbonitrile

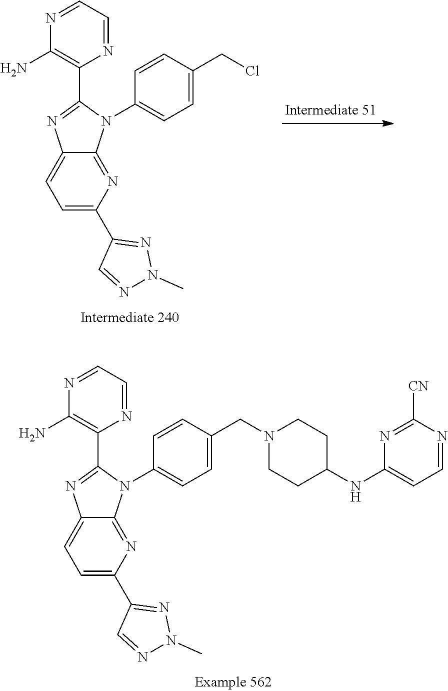 [SMILES: Cn1ncc(-c2ccc3nc(-c4nccnc4N)n(-c4ccc(CCl)cc4)c3n2)n1 ; Cn1ncc(-c2ccc3nc(-c4nccnc4N)n(-c4ccc(CN5CCC(Nc6ccnc(C#N)n6)CC5)cc4)c3n2)n1]

To a solution of Intermediate 240 (70 mg, 154 μmol, HCl salt) and Intermediate 51 (48.9 mg, 154 μmol, TFA salt) in DMF (2 mL) were added K₂CO₃ (106 mg, 770 μmol) and NaI (4.62 mg, 30.8 μmol). The mixture was stirred at 50° C. for 1 hr. The reaction mixture was quenched with H₂O (5 mL) at 25° C., diluted with CH₂Cl₂ (10 mL), and extracted with CH₂Cl₂ (15 mL×2). The combined organic layers were washed with brine (50 mL×3), dried over Na₂SO₄, filtered, and concentrated under reduced pressure. After purified by silica gel flash chromatography (Eluent of 1%˜6% MeOH in CH₂Cl₂), 4-((1-(4-(2-(3-aminopyrazin-2-yl)-5-(2-methyl-2H-1,2,3-triazol-4-yl)-3H-imidazo[4,5-b]pyridin-3-yl)benzyl)piperidin-4-yl)amino)pyrimidine-2-carbonitrile (Example 562, 43.2 mg, yield: 48% for three steps) was obtained as a yellow solid. MS: m/z=585.2 [M+H]⁺. ¹H NMR (400 MHz, Dimethylsulfoxide-d₆) δ 8.35 (d, J=8.0 Hz, 1H), 8.13-7.93 (m, 5H), 7.76 (br s, 2H), 7.48 (d, J=2.4 Hz, 1H), 7.44-7.40 (m, 2H), 7.38-7.33 (m, 2H), 6.68 (d, J=5.2 Hz, 1H), 4.21 (s, 3H), 3.90-3.76 (m, 1H), 3.60 (s, 2H), 2.93-2.82 (m, 2H), 2.25-2.12 (m, 2H), 1.98-1.84 (m, 2H), 1.59-1.43 (m, 2H).

### Example 563: 4-((1-(4-(2-(2-Aminopyridin-3-yl)-5-(3-methoxypyridin-2-yl)-3H-imidazo[4,5-b]pyridin-3-yl)benzyl)piperidin-4-yl)amino)pyrimidine-2-carbonitrile

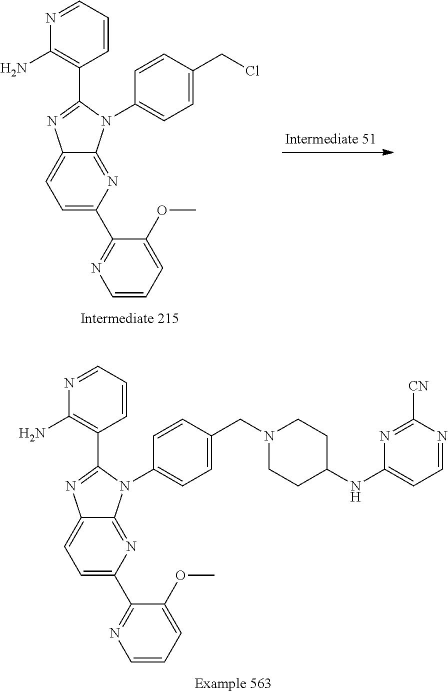 [SMILES: COc1cccnc1-c1ccc2nc(-c3cccnc3N)n(-c3ccc(CCl)cc3)c2n1 ; COc1cccnc1-c1ccc2nc(-c3cccnc3N)n(-c3ccc(CN4CCC(Nc5ccnc(C#N)n5)CC4)cc3)c2n1]

To a solution of Intermediate 215 (197 mg, 411 μmol, HCl salt) and Intermediate 51 (130 mg, 411 μmol, TFA salt) in DMF (4 mL) were added K₂CO₃ (284 mg, 2.05 mmol) and NaI (6.16 mg, 41.1 μmol). The mixture was stirred at 25° C. for 3 hr. The reaction mixture was quenched with H₂O (5 mL) at 25° C. and extracted with CH₂Cl₂ (15 mL×2). The combined organic layers were washed with brine (50 mL×3), dried over Na₂SO₄, filtered, and concentrated under reduced pressure. After purified byprep-HPLC (column: Phenomenex C18 150×25 mm×10 μm; mobile phase: [water (NH₄HCO₃)-ACN]; B %: 56%, isocratic elution mode), 4-((1-(4-(2-(2-aminopyridin-3-yl)-5-(3-methoxypyridin-2-yl)-3H-imidazo[4,5-b]pyridin-3-yl)benzyl)piperidin-4-yl)amino)pyrimidine-2-carbonitrile (Example 563, 83.2 mg, yield: 33% for two steps) was obtained as a light yellow lyophilized powder. MS: m/z=610.2 [M+H]⁺. ¹H NMR (400 MHz, Dimethylsulfoxide-d₆) δ 8.26-8.20 (m, 2H), 8.12-8.02 (m, 2H), 7.99 (dd, J=4.8, 2.0 Hz, 1H), 7.69 (d, J=8.0 Hz, 1H), 7.60-7.56 (m, 1H), 7.45-7.39 (m, 5H), 7.16 (dd, J=7.6, 1.6 Hz, 1H), 7.00 (br s, 2H), 6.66 (d, J=6.0 Hz, 1H), 6.38 (dd, J=7.6, 4.8 Hz, 1H), 3.88-3.78 (m, 1H), 3.76 (s, 3H), 3.55 (s, 2H), 2.84-2.75 (m, 2H), 2.17-2.07 (m, 2H), 1.89-1.80 (m, 2H), 1.51-1.40 (m, 2H).

### Example 564: 4-((1-(4-(2-(2-Aminopyridin-3-yl)-5-(1-methyl-1H-1,2,3-triazol-5-yl)-3H-imidazo[4,5-b]pyridin-3-yl)benzyl)piperidin-4-yl)amino)pyrimidine-2-carbonitrile

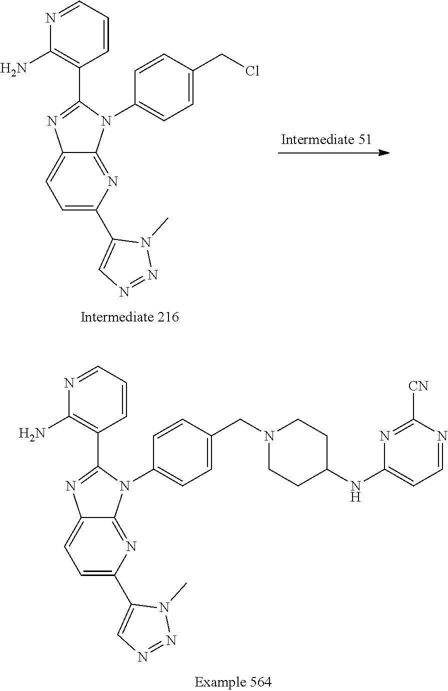 [SMILES: Cn1nncc1-c1ccc2nc(-c3cccnc3N)n(-c3ccc(CCl)cc3)c2n1 ; Cn1nncc1-c1ccc2nc(-c3cccnc3N)n(-c3ccc(CN4CCC(Nc5ccnc(C#N)n5)CC4)cc3)c2n1]

To a solution of Intermediate 216 (155 mg, 372 μmol, HCl salt) and Intermediate 51 (118 mg, 372 μmol, TFA) in DMF (2 mL) were added K₂CO₃ (257 mg, 1.86 mmol) and NaI (16.7 mg, 112 μmol). The mixture was stirred at 25° C. for 16 hr. The reaction mixture was poured into H₂O (15 mL) and extracted with EtOAc (15 mL×3). The combined organic layers were dried over anhydrous Na₂SO₄, filtered and concentrated under reduced pressure. After purified by silica gel flash chromatography (Eluent of 0%˜8% MeOH in CH₂Cl₂), 4-((1-(4-(2-(2-aminopyridin-3-yl)-5-(1-methyl-1H-1,2,3-triazol-5-yl)-3H-imidazo[4,5-b]pyridin-3-yl)benzyl)piperidin-4-yl)amino)pyrimidine-2-carbonitrile (Example 564, 44.3 mg, yield: 20% for three steps) was obtained as an off white solid. MS: m/z=584.2 [M+H]⁺. ¹HNMR (400 MHz, Dimethylsulfoxide-d₆) δ 8.40-8.31 (m, 2H), 8.14-7.97 (m, 3H), 7.89 (d, J=8.4 Hz, 1H), 7.55-7.40 (m, 4H), 7.24 (dd, J=7.6, 1.6 Hz, 1H), 7.09 (s, 2H), 6.67 (d, J=5.6 Hz, 1H), 6.41 (dd, J=8.0, 4.8 Hz, 1H), 4.16 (s, 3H), 3.89-3.72 (m, 1H), 3.58 (s, 2H), 2.85-2.73 (m, 2H), 2.20-2.08 (m, 2H), 1.97-1.81 (m, 2H), 1.57-1.41 (m, 2H).

### Example 565: 2-(2-Aminopyridin-3-yl)-3-(4-((4-((2-cyanopyrimidin-4-yl)amino)piperidin-1-yl)methyl)phenyl)-6-methyl-3H-imidazo[4,5-b]pyridine-5-carbonitrile

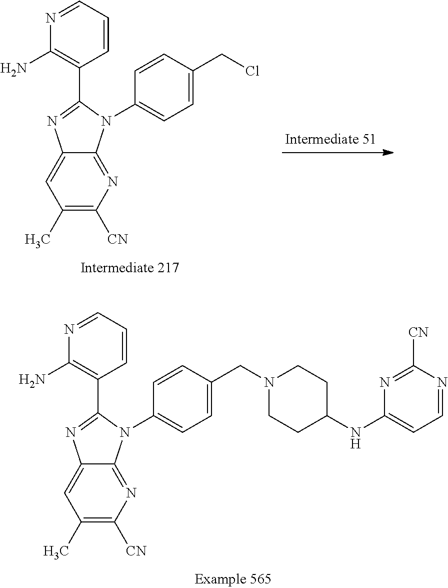 [SMILES: Cc1cc2nc(-c3cccnc3N)n(-c3ccc(CCl)cc3)c2nc1C#N ; Cc1cc2nc(-c3cccnc3N)n(-c3ccc(CN4CCC(Nc5ccnc(C#N)n5)CC4)cc3)c2nc1C#N]

To a solution of Intermediate 217 (28 mg, 68 μmol) and Intermediate 51 (21.6 mg, 68 μmol) in DMF (3 mL) were added NaI (5.1 mg, 34 μmol) and K₂CO₃ (38 mg, 2.7 μmol). The mixture was stirred at 25° C. for 16 hr. The reaction mixture was diluted with H₂O (10 mL) and extracted with EtOAc (10 mL×3). The combined organic layers were washed with brine (30 mL), dried over Na₂SO₄, filtered, and concentrated under reduced pressure. After purified by prep-TLC (CH₂Cl₂:MeOH=10:1), 2-(2-aminopyridin-3-yl)-3-(4-((4-((2-cyanopyrimidin-4-yl)amino)piperidin-1-yl)methyl)phenyl)-6-methyl-3H-imidazo[4,5-b]pyridine-5-carbonitrile (Example 565, 10.2 mg, yield: 28% for three steps) as a yellow solid. MS: m/z=542.1 [M+H]⁺. ¹H NMR (400 MHz, Methanol-d₄) δ 8.19 (s, 1H), 8.12-8.05 (m, 1H), 8.02 (dd, J=5.2, 2.0 Hz, 1H), 7.70 (d, J=8.0 Hz, 2H), 7.55 (d, J=8.0 Hz, 2H), 7.36 (dd, J=7.6, 1.6 Hz, 1H), 6.66 (d, J=6.0 Hz, 1H), 6.48 (dd, J=8.0, 5.2 Hz, 1H), 4.32-4.15 (m, 3H), 3.50-3.38 (m, 2H), 3.17-3.00 (m, 2H), 2.70 (s, 3H), 2.27-2.20 (m, 2H), 1.87-1.77 (m, 2H).

### Example 566: 4-((1-(4-(2-(2-Aminopyridin-3-yl)-5-(5-methyl-1,3,4-oxadiazol-2-yl)-3H-imidazo[4,5-b]pyridin-3-yl)benzyl)piperidin-4-yl)amino)pyrimidine-2-carbonitrile

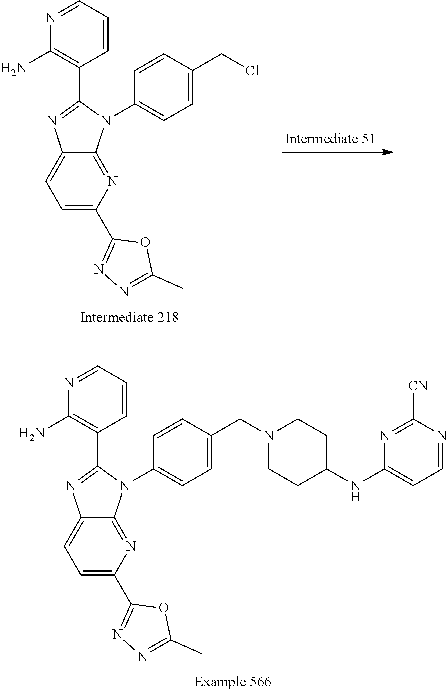 [SMILES: Cc1nnc(-c2ccc3nc(-c4cccnc4N)n(-c4ccc(CCl)cc4)c3n2)o1 ; Cc1nnc(-c2ccc3nc(-c4cccnc4N)n(-c4ccc(CN5CCC(Nc6ccnc(C#N)n6)CC5)cc4)c3n2)o1]

To a solution of Intermediate 218 (100 mg, 220 μmol, HCl salt) and Intermediate 51 (105 mg, 330 μmol, TFA salt) in DMF (2 mL) were added NaI (16.5 mg, 110 μmol) and K₂CO₃ (91 mg, 660 μmol). The mixture was stirred at 50° C. for 1 hr. The reaction mixture was diluted with H₂O (30 mL) and extracted with EtOAc (20 mL×3). The combined organic layers were washed with brine (30 mL), dried over Na₂SO₄, filtered and concentrated under reduced pressure. After purified by prep-HPLC (column: Phenomenex luna C18 150×25 mm×10 μm; mobile phase: [water (FA)-ACN]; gradient: 1%-30% B over 8 min), 4-((1-(4-(2-(2-aminopyridin-3-yl)-5-(5-methyl-1,3,4-oxadiazol-2-yl)-3H-imidazo[4,5-b]pyridin-3-yl)benzyl)piperidin-4-yl)amino)pyrimidine-2-carbonitrile (Example 566, 7.3 mg, yield: 6%) was obtained as an off white solid. MS: m/z=585.2 [M+H]⁺. ¹H NMR (400 MHz, Chloroform-d) δ 8.35-8.27 (m, 1H), 8.27-8.20 (m, 2H), 8.19-8.10 (m, 1H), 8.06 (dd, J=4.8, 1.6 Hz, 1H), 7.54 (d, J=8.0 Hz, 2H), 7.41 (d, J=8.0 Hz, 2H), 7.08 (dd, J=7.6, 1.2 Hz, 1H), 6.95-6.83 (m, 2H), 6.50-6.43 (m, 1H), 6.36 (dd, J=7.6, 4.8 Hz, 1H), 5.47-5.24 (m, 1H), 3.74 (s, 2H), 3.11-2.94 (m, 2H), 2.63 (s, 3H), 2.35-2.28 (m, 2H), 2.09 (d, J=10.4 Hz, 2H), 1.75-1.62 (m, 2H).

### Example 567: 4-((1-(4-(2-(2-Aminopyridin-3-yl)-5-(1-methyl-1H-1,2,4-triazol-5-yl)-3H-imidazo[4,5-b]pyridin-3-yl)benzyl)piperidin-4-yl)amino)pyrimidine-2-carbonitrile

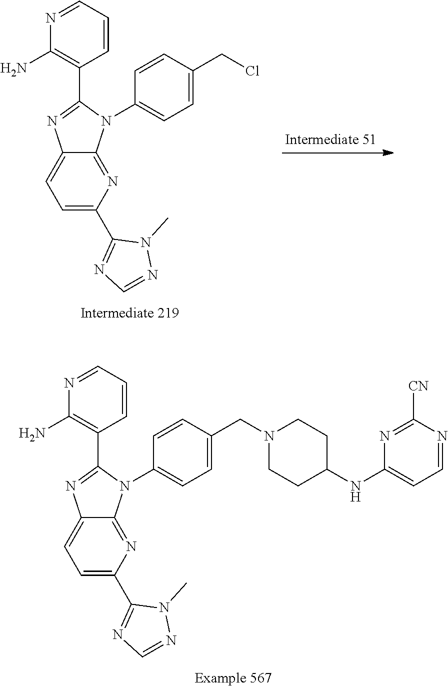 [SMILES: Cn1ncnc1-c1ccc2nc(-c3cccnc3N)n(-c3ccc(CCl)cc3)c2n1 ; Cn1ncnc1-c1ccc2nc(-c3cccnc3N)n(-c3ccc(CN4CCC(Nc5ccnc(C#N)n5)CC4)cc3)c2n1]

To a solution of Intermediate 219 (180 mg, 432 μmol, HCl salt) and Intermediate 51 (140 mg, 432 μmol, TFA) in DMF (2 mL) were added K₂CO₃ (298 mg, 2.16 mmol) and NaI (19.4 mg, 130 μmol). The mixture was stirred at 25° C. for 16 hr. The reaction mixture was poured into H₂O (15 mL) and extracted with EtOAc (15 mL×3). The organic phase was dried over anhydrous Na₂SO₄, filtered, and concentrated under reduced pressure. After purified by silica gel flash chromatography (Eluent of 0%˜8% MeOH in CH₂Cl₂), 4-((1-(4-(2-(2-aminopyridin-3-yl)-5-(1-methyl-1H-1,2,4-triazol-5-yl)-3H-imidazo[4,5-b]pyridin-3-yl)benzyl)piperidin-4-yl)amino)pyrimidine-2-carbonitrile (Example 567, 87.7 mg, yield: 35% for three steps) was obtained as a yellow solid. MS: m/z=584.2 [M+H]⁺. ¹H NMR (400 MHz, Dimethylsulfoxide-d₆) δ 8.37 (d, J=8.4 Hz, 1H), 8.16 (d, J=8.4 Hz, 1H), 8.11-7.98 (m, 4H), 7.51-7.40 (m, 4H), 7.26 (dd, J=7.6, 1.6 Hz, 1H), 7.09 (s, 2H), 6.67 (d, J=6.0 Hz, 1H), 6.42 (dd, J=7.6, 4.8 Hz, 1H), 4.08 (s, 3H), 3.88-3.72 (m, 1H), 3.58 (s, 2H), 2.85-2.73 (m, 2H), 2.19-2.09 (m, 2H), 1.93-1.82 (m, 2H), 1.53-1.42 (m, 2H).

### Example 568: 4-((1-(4-(2-(2-Aminopyridin-3-yl)-5-(4-methyl-2H-1,2,3-triazol-2-yl)-3H-imidazo[4,5-b]pyridin-3-yl)benzyl)piperidin-4-yl)amino)pyrimidine-2-carbonitrile

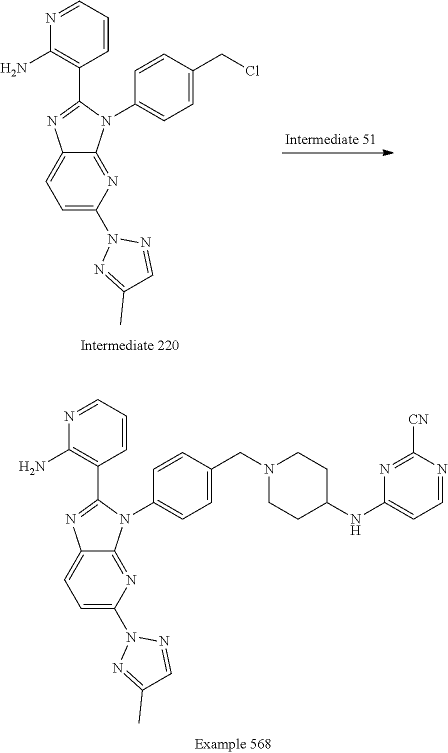 [SMILES: Cc1cnn(-c2ccc3nc(-c4cccnc4N)n(-c4ccc(CCl)cc4)c3n2)n1 ; Cc1cnn(-c2ccc3nc(-c4cccnc4N)n(-c4ccc(CN5CCC(Nc6ccnc(C#N)n6)CC5)cc4)c3n2)n1]

To a solution of Intermediate 220 (170 mg, 375 μmol, HCl salt) and Intermediate 51 (119 mg, 375 μmol, TFA) in DMF (5 mL) were added K₂CO₃ (259 mg, 1.88 mmol) and NaI (5.62 mg, 37.5 μmol). The mixture was stirred at 50° C. for 1 hr. The reaction mixture was quenched with H₂O (50 mL) at 25° C. and extracted with CH₂Cl₂ (50 mL×2). The combined organic layers were washed with brine (50 mL×3), dried over Na₂SO₄, filtered, and concentrated under reduced pressure. After purified by silica gel flash chromatography (Eluent of 1%˜10% MeOH in CH₂Cl₂), 4-((1-(4-(2-(2-aminopyridin-3-yl)-5-(4-methyl-2H-1,2,3-triazol-2-yl)-3H-imidazo[4,5-b]pyridin-3-yl)benzyl)piperidin-4-yl)amino)pyrimidine-2-carbonitrile (Example 568, 105.2 mg, yield: 47% for two steps) was obtained as a pink solid. MS: m/z=584.2 [M+H]⁺. ¹H NMR (400 MHz, Dimethylsulfoxide-d₆) δ 8.40 (d, J=8.4 Hz, 1H), 8.12-7.96 (m, 4H), 7.90 (s, 1H), 7.52-7.43 (m, 4H), 7.15 (dd, J=7.6, 1.6 Hz, 1H), 6.95 (s, 2H), 6.68 (d, J=5.6 Hz, 1H), 6.41-6.35 (m, 1H), 3.94-3.68 (m, 1H), 3.59 (s, 2H), 2.87-2.79 (m, 2H), 2.36 (s, 3H), 2.22-2.13 (m, 2H), 1.93-1.85 (m, 2H), 1.59-1.48 (m, 2H).

### Example 569: 4-((1-(4-(2-(2-Aminopyridin-3-yl)-5-(4-methyl-4H-1,2,4-triazol-3-yl)-3H-imidazo[4,5-b]pyridin-3-yl)benzyl)piperidin-4-yl)amino)pyrimidine-2-carbonitrile

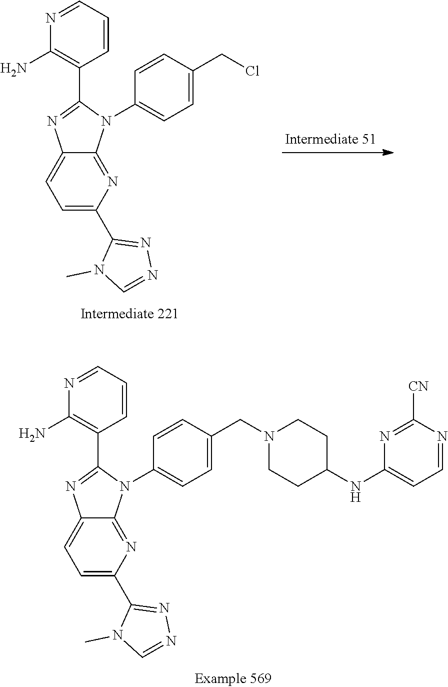 [SMILES: Cn1cnnc1-c1ccc2nc(-c3cccnc3N)n(-c3ccc(CCl)cc3)c2n1 ; Cn1cnnc1-c1ccc2nc(-c3cccnc3N)n(-c3ccc(CN4CCC(Nc5ccnc(C#N)n5)CC4)cc3)c2n1]

To a solution of Intermediate 221 (160 mg, 384 μmol, HCl salt) and Intermediate 51 (122 mg, 384 μmol, TFA salt) in DMF (2 mL) were added K₂CO₃ (265 mg, 1.92 mmol) and NaI (17.3 mg, 115 μmol). The mixture was stirred at 25° C. for 16 hr. The reaction mixture was poured into H₂O (15 mL) and extracted with EtOAc (15 mL×3). The combined organic phases were dried over anhydrous Na₂SO₄, filtered and concentrated under reduced pressure. After purified by silica gel flash chromatography (Eluent of 0%˜6% MeOH in CH₂Cl₂), 4-((1-(4-(2-(2-aminopyridin-3-yl)-5-(4-methyl-4H-1,2,4-triazol-3-yl)-3H-imidazo[4,5-b]pyridin-3-yl)benzyl)piperidin-4-yl)amino)pyrimidine-2-carbonitrile (Example 569, 20.1 mg, yield: 9% for three steps) was obtained as a light brown solid. MS: m/z=584.2 [M+H]⁺. ¹H NMR (400 MHz, Dimethylsulfoxide-d₆) δ 8.53 (s, 1H), 8.36 (d, J=8.4 Hz, 1H), 8.17 (d, J=8.4 Hz, 1H), 8.12-7.99 (m, 3H), 7.49-7.45 (m, 4H), 7.25 (dd, J=8.0 Hz, 2.0 Hz, 1H), 7.08 (s, 2H), 6.67 (d, J=6.0 Hz, 1H), 6.41 (dd, J=7.6 Hz, 4.8 Hz, 1H), 3.85-3.81 (m, 1H), 3.80 (s, 3H), 3.58 (s, 2H), 2.83-2.75 (m, 2H), 2.20-2.08 (m, 2H), 1.95-1.83 (m, 2H), 1.54-1.43 (m, 2H).

### Example 570: 4-((1-(4-(2-(2-Aminopyridin-3-yl)-5-(3-fluoropyridin-2-yl)-3H-imidazo[4,5-b]pyridin-3-yl)benzyl)piperidin-4-yl)amino)pyrimidine-2-carbonitrile

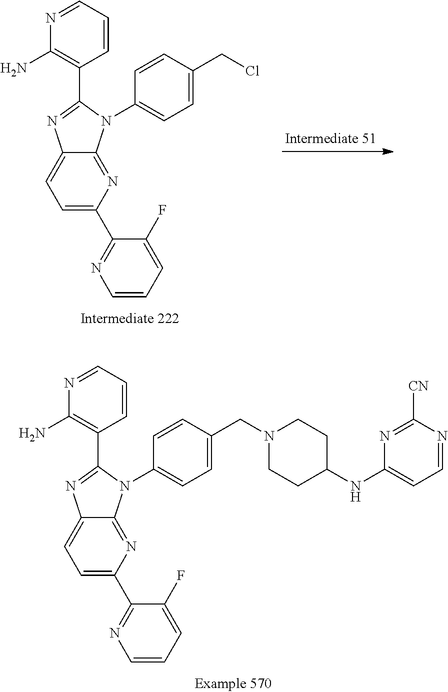 [SMILES: Nc1ncccc1-c1nc2ccc(-c3ncccc3F)nc2n1-c1ccc(CCl)cc1 ; N#Cc1nccc(NC2CCN(Cc3ccc(-n4c(-c5cccnc5N)nc5ccc(-c6ncccc6F)nc54)cc3)CC2)n1]

To a mixture of Intermediate 222 (200 mg, 464 μmol) and Intermediate 51 (141 mg, 696 μmol) in DMF (3 mL) was added DIEA (179 mg, 1.39 mmol). The mixture was stirred at 80° C. for 1 hr. The mixture was diluted with H₂O (20 mL) and extracted with EtOAc (20 mL×2). The combined organic layers were washed with brine (20 mL), dried over anhydrous Na₂SO₄, filtered and concentrated. After purified by prep-HPLC (column: C18 150-30 mm; mobile phase: [water (NH₃H₂O+NH₄HCO₃)-ACN]; gradient: 40%-70% B over 7 min), 4-((I-(4-(2-(2-aminopyridin-3-yl)-5-(3-fluoropyridin-2-yl)-3H-imidazo[4,5-b]pyridin-3-yl)benzyl)piperidin-4-yl)amino)pyrimidine-2-carbonitrile (Example 570, 30.7 mg, yield: 11%) was obtained as a yellow solid. MS: m/z=620.1 [M+Na]. ¹H NMR (400 MHz, Dimethylsulfoxide-d₆) δ 8.53 (d, J=4.8 Hz, 1H), 8.34 (d, J=8.0 Hz, 1H), 8.11-7.98 (m, 4H), 7.86-7.76 (m, 1H), 7.59-7.50 (m, 1H), 7.48-7.39 (m, 4H), 7.18 (d, J=7.6, 1H), 6.98 (br s, 2H), 6.72-6.64 (m, 1H), 6.39 (dd, J=7.6, 4.8 Hz, 1H), 3.86-3.77 (m, 1H), 3.56 (s, 2H), 2.87-2.75 (m, 2H), 2.21-2.09 (m, 2H), 1.94-1.79 (m, 2H), 1.56-1.41 (m, 2H). ¹⁹F NMR (400 MHz, Dimethylsulfoxide-d) 5-121.825.

### Example 571: 4-((1-(4-(2-(2-Aminopyridin-3-yl)-5-(6-methylpyridin-2-yl)-3H-imidazo[4,5-b]pyridin-3-yl)benzyl)piperidin-4-yl)amino)pyrimidine-2-carbonitrile

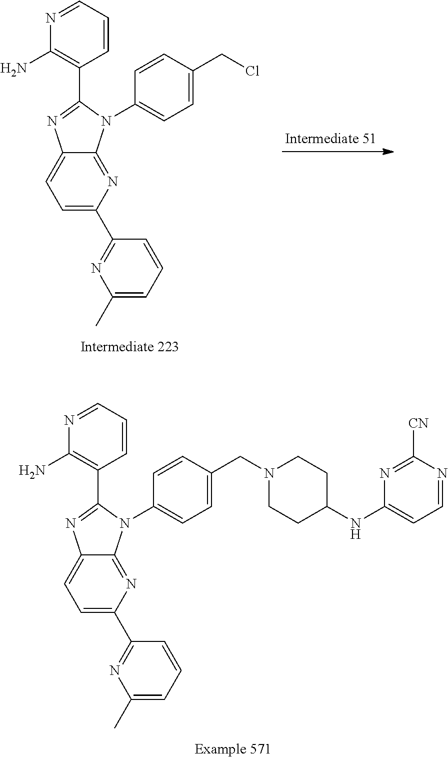 [SMILES: Cc1cccc(-c2ccc3nc(-c4cccnc4N)n(-c4ccc(CCl)cc4)c3n2)n1 ; Cc1cccc(-c2ccc3nc(-c4cccnc4N)n(-c4ccc(CN5CCC(Nc6ccnc(C#N)n6)CC5)cc4)c3n2)n1]

To a mixture of Intermediate 223 (240 mg, 562 μmol) and Intermediate 51 (171 mg, 843 μmol) in DMF (2 mL) was added DIEA (218 mg, 1.69 mmol). The mixture was stirred at 80° C. for 1 hr. The mixture was diluted with H₂O (20 mL) and extracted with EtOAc (20 mL×2). The combined organic layers were washed with brine (20 mL), dried over anhydrous Na₂SO₄, filtered and concentrated. After purified by prep-HPLC (column: C18 150×30 mm; mobile phase: [water (NH₃H₂O+NH₄HCO₃)-ACN]; gradient: 50%-80% B over 7 min), 4-((1-(4-(2-(2-aminopyridin-3-yl)-5-(6-methylpyridin-2-yl)-3H-imidazo[4,5-b]pyridin-3-yl)benzyl)piperidin-4-yl)amino)pyrimidine-2-carbonitrile (Example 571, 32 mg, yield: 9%) was obtained as a yellow solid. MS: m/z=616.1 [M+Na]⁺. ¹H NMR (400 MHz, Dimethylsulfoxide-d₆) δ 8.48 (d, J=8.4 Hz, 1H), 8.29 (d, J=8.4 Hz, 1H), 8.23-7.86 (m, 4H), 7.77 (t, J=8.0 Hz, 1H), 7.53-7.41 (m, 4H), 7.26 (d, J=7.6 Hz, 1H), 7.17 (dd, J=7.6, 1.6 Hz, 1H), 7.01 (br s, 2H), 6.67 (d, J=6.0 Hz, 1H), 6.39 (dd, J=7.6, 4.4 Hz, 1H), 3.90-3.75 (m, 1H), 3.60 (s, 2H), 2.92-2.78 (m, 2H), 2.57 (s, 3H), 2.24-2.11 (m, 2H), 1.95-1.76 (m, 2H), 1.60-1.40 (m, 2H).

### Example 572: 4-((1-(4-(2-(2-Aminopyridin-3-yl)-5-(1-(difluoromethyl)-1H-1,2,4-triazol-3-yl)-3H-imidazo[4,5-b]pyridin-3-yl)benzyl)piperidin-4-yl)amino)pyrimidine-2-carbonitrile

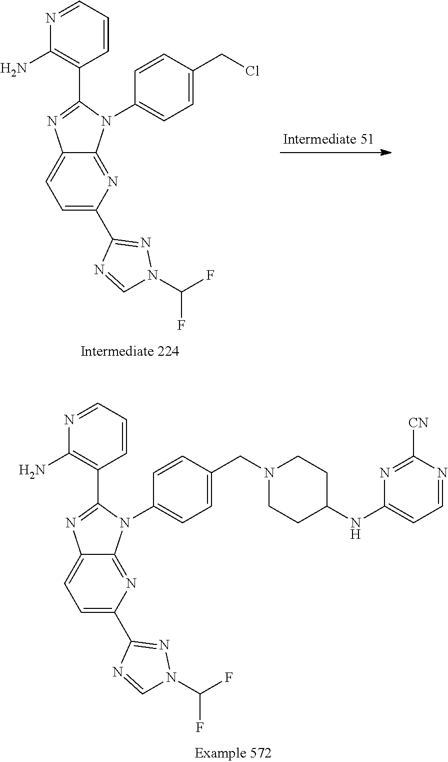 [SMILES: Nc1ncccc1-c1nc2ccc(-c3ncn(C(F)F)n3)nc2n1-c1ccc(CCl)cc1 ; N#Cc1nccc(NC2CCN(Cc3ccc(-n4c(-c5cccnc5N)nc5ccc(-c6ncn(C(F)F)n6)nc54)cc3)CC2)n1]

To a solution of Intermediate 224 (52 mg, 115 μmol, HCl salt) and Intermediate 51 (36.4 mg, 115 μmol, TFA) in DMF (2 mL) were added K₂CO₃ (79.4 mg, 574 μmol) and NaI (1.72 mg, 11.5 μmol). The mixture was stirred at 50° C. for 1 hr. The reaction mixture was quenched with H₂O (50 mL) at 25° C. and extracted with CH₂Cl₂ (50 mL×2). The combined organic layers were washed with brine (50 mL×3), dried over Na₂SO₄, filtered, and concentrated under reduced pressure. After purified by silica gel flash chromatography (Eluent of 1%˜8% MeOH in CH₂Cl₂), 4-((1-(4-(2-(2-aminopyridin-3-yl)-5-(1-(difluoromethyl)-1H-1,2,4-triazol-3-yl)-3H-imidazo[4,5-b]pyridin-3-yl)benzyl)piperidin-4-yl)amino)pyrimidine-2-carbonitrile (Example 572, 26.1 mg, yield: 36% for two steps) was obtained as an off white solid. MS: m/z=620.1 [M+H]⁺. ¹H NMR (400 MHz, Dimethylsulfoxide-d₆) δ 9.18 (s, 1H), 8.34 (d, J=8.4 Hz, 1H), 8.19 (d, J=8.4 Hz, 1H), 8.11-7.84 (m, 4H), 7.51-7.41 (m, 4H), 7.19-7.12 (m, 1H), 6.99 (s, 2H), 6.68 (d, J=6.0 Hz, 1H), 6.40-6.35 (m, 1H), 3.88-3.77 (m, 1H), 3.59 (s, 2H), 2.89-2.81 (m, 2H), 2.23-2.13 (m, 2H), 1.95-1.84 (m, 2H), 1.54-1.43 (m, 2H). ¹⁹F NMR (400 MHz, Dimethylsulfoxide-d₆) δ −96.949.

### Example 573: 4-((1-(4-(2-(4-Aminopyrimidin-5-yl)-5-(5-fluoropyridin-2-yl)-3H-imidazo[4,5-b]pyridin-3-yl)benzyl)piperidin-4-yl)amino)pyrimidine-2-carbonitrile

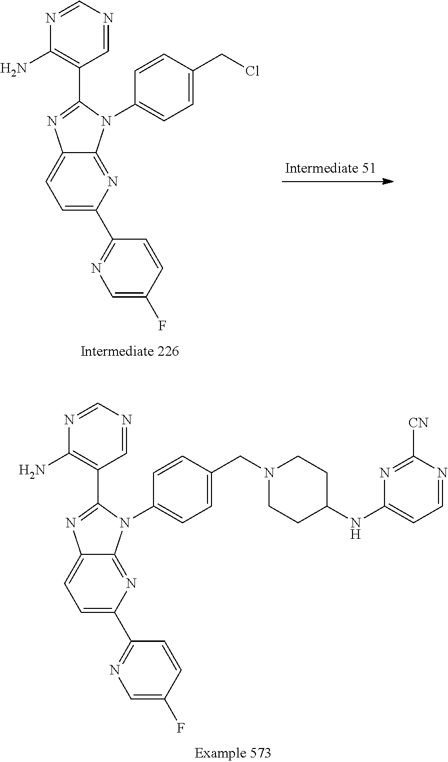 [SMILES: Nc1ncncc1-c1nc2ccc(-c3ccc(F)cn3)nc2n1-c1ccc(CCl)cc1 ; N#Cc1nccc(NC2CCN(Cc3ccc(-n4c(-c5cncnc5N)nc5ccc(-c6ccc(F)cn6)nc54)cc3)CC2)n1]

To a solution of Intermediate 226 (509 mg, 1.09 mmol, HCl salt) and Intermediate 51 (345 mg, 1.09 mmol, TFA) in DMF (5 mL) were added K₂CO₃ (751 mg, 5.43 mmol) and NaI (16.3 mg, 109 μmol). The mixture was stirred at 50° C. for 1 hr. The reaction mixture was quenched with H₂O (50 mL) at 25° C. and extracted with CH₂Cl₂ (50 mL×2). The combined organic layers were washed with brine (50 mL×3), dried over Na₂SO₄, filtered, and concentrated under reduced pressure. After purified by silica gel flash chromatography (Eluent of 1%˜5% MeOH in CH₂Cl₂), 4-((1-(4-(2-(4-aminopyrimidin-5-yl)-5-(5-fluoropyridin-2-yl)-3H-imidazo[4,5-b]pyridin-3-yl)benzyl)piperidin-4-yl)amino)pyrimidine-2-carbonitrile (Example 573, 273.5 mg, yield: 40% for two steps) was obtained as a brown solid. MS: m/z=599.2 [M+H]⁺. ¹H NMR (400 MHz, Dimethylsulfoxide-d₆) δ 8.66 (d, J=2.0 Hz, 1H), 8.53-8.29 (m, 3H), 8.25-8.19 (m, 1H), 8.17-7.99 (m, 2H), 7.94 (s, 1H), 7.85-7.66 (m, 3H), 7.52 (s, 4H), 6.87-6.62 (m, 1H), 3.98-3.67 (m, 1H), 3.60 (s, 2H), 2.89-2.78 (m, 2H), 2.26-2.12 (m, 2H), 1.95-1.85 (m, 2H), 1.57-1.43 (m, 2H). ¹⁹F NMR (400 MHz, Dimethylsulfoxide-d₆) δ −127.659.

### Example 574: 4-((1-(4-(2-(3-Aminopyrazin-2-yl)-5-(5-fluoropyridin-2-yl)-3H-imidazo[4,5-b]pyridin-3-yl)benzyl)piperidin-4-yl)amino)pyrimidine-2-carbonitrile

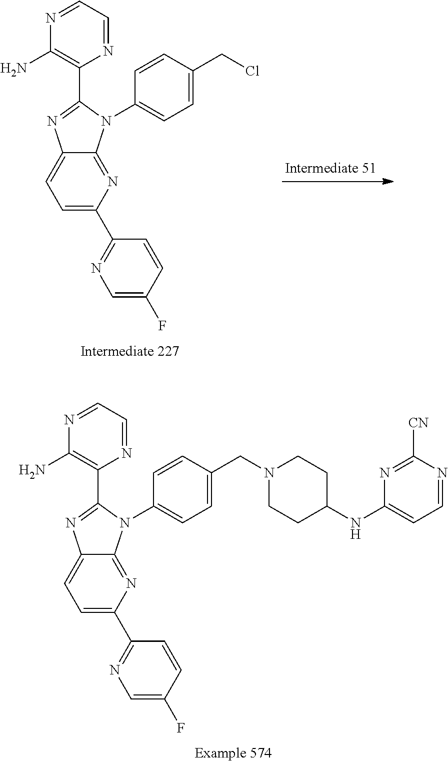 [SMILES: Nc1nccnc1-c1nc2ccc(-c3ccc(F)cn3)nc2n1-c1ccc(CCl)cc1 ; N#Cc1nccc(NC2CCN(Cc3ccc(-n4c(-c5nccnc5N)nc5ccc(-c6ccc(F)cn6)nc54)cc3)CC2)n1]

To a solution of Intermediate 227 (238 mg, 508 μmol, HCl salt) and Intermediate 51 (161 mg, 508 μmol, TFA) in DMF (3 mL) were added NaI (38 mg, 254 μmol) and K₂CO₃ (281 mg, 2.03 mmol). The mixture was stirred at 25° C. for 16 hr. The reaction mixture was diluted with H₂O (10 mL) and extracted with EtOAc (10 mL×3). The combined organic layers were washed with brine (30 mL), dried over Na₂SO₄, filtered, and concentrated under reduced pressure. After purified by prep-TLC (CH₂Cl₂:MeOH=10:1), 4-((1-(4-(2-(3-aminopyrazin-2-yl)-5-(5-fluoropyridin-2-yl)-3H-imidazo[4,5-b]pyridin-3-yl)benzyl)piperidin-4-yl)amino)pyrimidine-2-carbonitrile (Example 574, 36 mg, yield: 12% for three steps) was obtained as a yellow solid. MS: m/z=599.0 [M+H]⁺. ¹H NMR (400 MHz, Dimethysulfoxide-d₆) δ 8.67 (d, J=2.4 Hz, 1H), 8.48-8.33 (m, 2H), 8.20 (dd, J=8.8, 4.8 Hz, 1H), 8.13-8.04 (m, 2H), 8.02 (d, J=2.0 Hz, 1H), 7.90-7.73 (m, 3H), 7.49 (d, J=2.4 Hz, 1H), 7.47-7.37 (m, 4H), 6.68 (d, J=6.0 Hz, 1H), 3.95-3.75 (m, 1H), 3.61 (s, 2H), 2.94-2.81 (m, 2H), 2.24-2.14 (m, 2H), 1.97-1.86 (m, 2H), 1.55-1.47 (m, 2H). ¹⁹F NMR (400 MHz, Dimethylsulfoxide-d₆) δ −127.539.

### Example 575: 4-((1-(4-(5-(5-Fluoropyridin-2-yl)-2-(pyridin-3-yl)-3H-imidazo[4,5-b]pyridin-3-yl)benzyl)piperidin-4-yl)amino)pyrimidine-2-carbonitrile

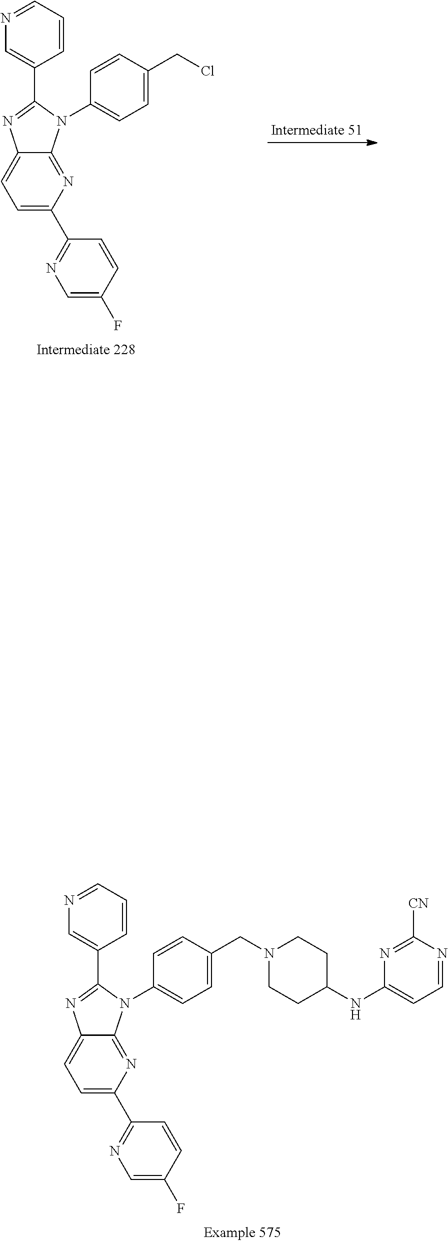 [SMILES: Fc1ccc(-c2ccc3nc(-c4cccnc4)n(-c4ccc(CCl)cc4)c3n2)nc1 ; N#Cc1nccc(NC2CCN(Cc3ccc(-n4c(-c5cccnc5)nc5ccc(-c6ccc(F)cn6)nc54)cc3)CC2)n1]

To a solution of Intermediate 228 (410 mg, 906 μmol, HCl salt) and Intermediate 51 (288 mg, 906 μmol, TFA) in DMF (5 mL) were added NaI (68 mg, 453 μmol) and K₂CO₃ (501 mg, 3.63 mmol). The mixture was stirred at 25° C. for 16 hr. The reaction mixture was diluted with H₂O (10 mL) and extracted with EtOAc (10 mL×3). The combined organic layers were washed with brine (30 mL), dried over Na₂SO₄, filtered, and concentrated under reduced pressure. After purified by prep-HPLC (column: Phenomenex C18 150×25 mm×10 μm; mobile phase: [water (NH₄HCO₃)-ACN]; B %: 38%, isocratic elution mode), 4-((1-(4-(5-(5-fluoropyridin-2-yl)-2-(pyridin-3-yl)-3H-imidazo[4,5-b]pyridin-3-yl)benzyl)piperidin-4-yl)amino)pyrimidine-2-carbonitrile (Example 575, 208 mg, yield: 40% for three steps) was obtained as a white powder. MS: m/z=583.1 [M+H]⁺. ¹H NMR (400 MHz, Dimethysulfoxide-d₆) δ 8.81-8.53 (m, 3H), 8.47-8.33 (m, 2H), 8.25 (dd, J=8.8, 4.8 Hz, 1H), 8.20-7.98 (m, 2H), 7.94-7.80 (m, 2H), 7.57-7.48 (m, 4H), 7.45 (dd, J=8.0, 4.8 Hz, 1H), 6.85-6.65 (m, 1H), 3.92-3.74 (m, 1H), 3.62 (s, 2H), 2.90-2.82 (m, 2H), 2.23-2.12 (m, 2H), 1.97-1.84 (m, 2H), 1.58-1.45 (m, 2H). ¹⁹F NMR (400 MHz, Dimethylsulfoxide-d₆) δ −127.614.

### Example 576: 4-((1-(4-(2-(2-Aminophenyl)-5-(5-fluoropyridin-2-yl)-3H-imidazo[4,5-b]pyridin-3-yl)benzyl)piperidin-4-yl)amino)pyrimidine-2-carbonitrile

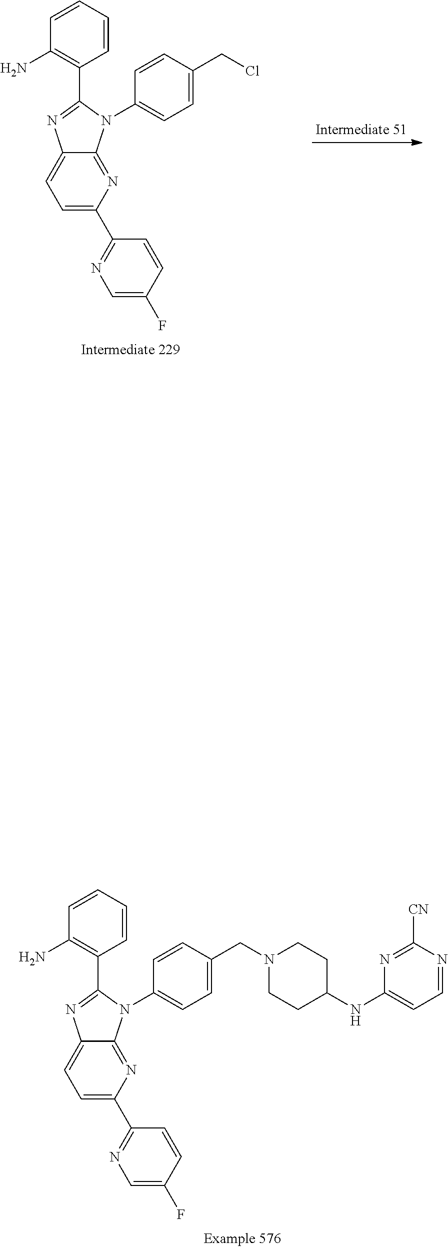 [SMILES: Nc1ccccc1-c1nc2ccc(-c3ccc(F)cn3)nc2n1-c1ccc(CCl)cc1 ; N#Cc1nccc(NC2CCN(Cc3ccc(-n4c(-c5ccccc5N)nc5ccc(-c6ccc(F)cn6)nc54)cc3)CC2)n1]

To a solution of Intermediate 229 (175 mg, 407 μmol, HCl salt) and Intermediate 51 (124 mg, 611 μmol, TFA salt) in DMF (3 mL) were added NaI (61 mg, 407 μmol) and K₂CO₃ (169 mg, 1.22 mmol). The mixture was stirred at 25° C. for 16 hr. The reaction mixture was diluted with H₂O (30 mL) and extracted with EtOAc (20 mL×3). The combined organic layers were washed with brine (30 mL), dried over Na₂SO₄, filtered, and concentrated under reduced pressure. After purified by silica gel flash chromatography (Eluent of 0˜9% MeOH in CH₂Cl₂), 4-((1-(4-(2-(2-aminophenyl)-5-(5-fluoropyridin-2-yl)-3H-imidazo[4,5-b]pyridin-3-yl)benzyl)piperidin-4-yl)amino)pyrimidine-2-carbonitrile (Example 576, 38.7 mg, yield: 16% for three steps) was obtained as a yellow solid. MS: m/z=597.2 [M+H]⁺. ¹H NMR (400 MHz, Dimethysulfoxide-d₆) δ8.66 (d, J=2.8 Hz, 1H), 8.42-8.35 (m, 1H), 8.30-8.19 (m, 2H), 8.14-8.04 (m, 2H), 7.90-7.77 (m, 1H), 7.50-7.38 (m, 4H), 7.12-7.01 (m, 1H), 6.81 (d, J=8.0 Hz, 2H), 6.68 (d, J=6.0 Hz, 1H), 6.32-6.28 (m, 2H), 3.94-3.72 (m, 1H), 3.59 (s, 2H), 2.83 (d, J=10.8 Hz, 2H), 2.27-2.05 (m, 2H), 1.96-1.81 (m, 2H), 1.60-1.40 (m, 2H). ¹⁹F NMR (400 MHz, Dimethysulfoxide-dₕ) 6-127.98.

### Example 577: 4-((1-(4-(5-(5-Fluoropyridin-2-yl)-2-phenyl-3H-imidazo[4,5-h]pyridin-3-yl)benzyl)piperidin-4-yl)amino)pyrimidine-2-carbonitrile

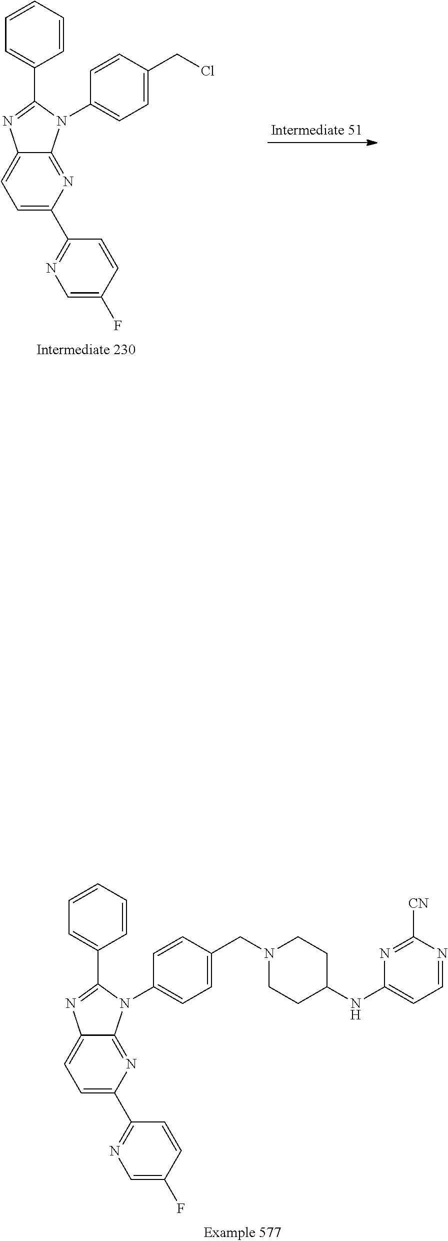 [SMILES: Fc1ccc(-c2ccc3nc(-c4ccccc4)n(-c4ccc(CCl)cc4)c3n2)nc1 ; N#Cc1nccc(NC2CCN(Cc3ccc(-n4c(-c5ccccc5)nc5ccc(-c6ccc(F)cn6)nc54)cc3)CC2)n1]

To a solution of Intermediate 230 (103 mg, 248 μmol, HCl salt) and Intermediate 51 (78.8 mg, 248 μmol, TFA) in DMF (2 mL) were added K₂CO₃ (103 mg, 745 μmol) and NaI (7.44 mg, 49.7 μmol). The mixture was stirred at 50° C. for 0.5 hr. The reaction mixture was quenched with H₂O (50 mL) at 25° C. and extracted with CH₂Cl₂ (50 mL×3). The combined organic layers were washed with brine (50 mL×3), dried over Na₂SO₄, filtered and concentrated under reduced pressure. After purified byprep-HPLC (column: Waters xbridge 150×25 mm 10 μm; mobile phase: [water (NH₄HCO₃)-ACN]; gradient: 42%-72% B over 10 min), 4-((1-(4-(5-(5-fluoropyridin-2-yl)-2-phenyl-3H-imidazo[4,5-b]pyridin-3-yl)benzyl)piperidin-4-yl)amino)pyrimidine-2-carbonitrile (Example 577, 23.9 mg, yield: 17% for two steps) was obtained as a light yellow lyophilized powder. MS: m/z=582.2 [M+H]⁺. ¹H NMR (400 MHz, Dimethylsulfoxide-d₆) δ 8.52 (d, J=2.8 Hz, 1H), 8.47 (d, J=8.4 Hz, 1H), 8.34 (dd, J=8.8, 4.4 Hz, 1H), 8.23 (d, J=8.4 Hz, 1H), 8.06-7.95 (m, 1H), 7.63-7.55 (m, 5H), 7.48-7.44 (m, 3H), 7.40 (d, J=7.6 Hz, 2H), 6.60 (d, J=4.8 Hz, 1H), 4.08-3.85 (m, 1H), 3.69 (s, 2H), 3.02-2.98 (m, 2H), 2.34-2.27 (m, 2H), 2.04-1.99 (m, 2H), 1.67-1.58 (m, 2H).

### Example 578: 4-((1-(4-(2-(2-Aminopyridin-3-yl)-5-(5-(methoxy-d₃)pyridin-2-yl)-3H-imidazo[4,5-b]pyridin-3-yl)benzyl)piperidin-4-yl)amino)pyrimidine-2-carbonitrile

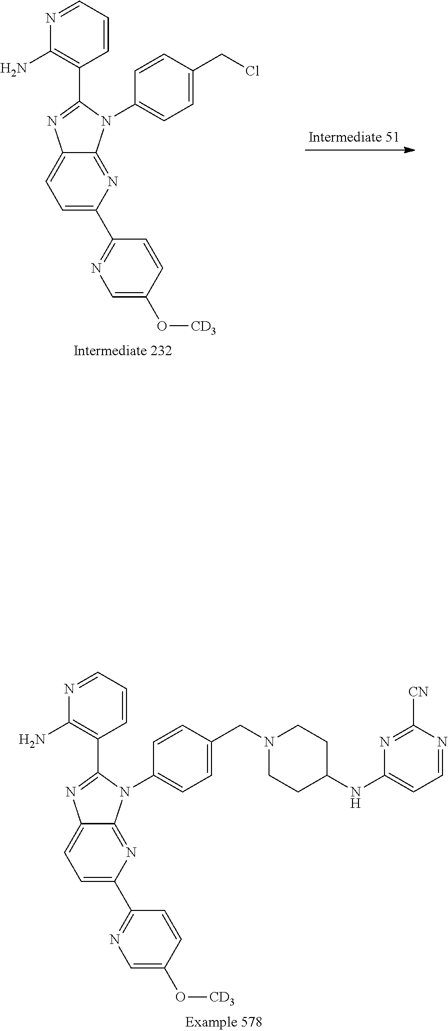 [SMILES: [2H]C([2H])([2H])Oc1ccc(-c2ccc3nc(-c4cccnc4N)n(-c4ccc(CCl)cc4)c3n2)nc1 ; [2H]C([2H])([2H])Oc1ccc(-c2ccc3nc(-c4cccnc4N)n(-c4ccc(CN5CCC(Nc6ccnc(C#N)n6)CC5)cc4)c3n2)nc1]

To a solution of Intermediate 232 (150 mg, 446 μmol) and Intermediate 51 (82 mg, 404 μmol) in DMF (3 mL) were added K₂CO₃ (186 mg, 1.3 mmol) and NaI (2.5 mg, 17 μmol). The mixture was stirred at 50° C. for 1 hr. The reaction mixture was quenched with H₂O (10 mL) at 25° C. and extracted with CH₂Cl₂ (15 mL×2). The combined organic layers were washed with brine (50 mL×3), dried over Na₂SO₄, filtered and concentrated under reduced pressure. After purified by silica gel flash chromatography (Eluent of 0˜5% MeOH in CH₂Cl₂), 4-((1-(4-(2-(2-aminopyridin-3-yl)-5-(5-(methoxy-d₃)pyridin-2-yl)-3H-imidazo[4,5-b]pyridin-3-yl)benzyl)piperidin-4-yl)amino)pyrimidine-2-carbonitrile (Example 578, 22.5 mg, yield: 11% for three steps) was obtained as a yellow solid. MS: m/z=613.2 [M+H]⁺. D %: 3D=99.3%. ¹H NMR (400 MHz, Dimethylsulfoxide-d₆) δ 8.39-8.34 (m, 2H), 8.27 (d, J=8.4 Hz, 1H), 8.14 (d, J=8.8 Hz, 1H), 8.07-8.02 (m, 2H), 8.00 (dd, 1=4.8, 1.6 Hz, 1H), 7.51-7.44 (m, 5H), 7.16 (dd, J=7.6, 1.6 Hz, 1H), 7.02 (s, 2H), 6.68 (d, J=6.0 Hz, 1H), 6.38 (dd, J=7.6, 4.8 Hz, 1H), 3.92-3.72 (m, 1H), 3.60 (s, 2H), 2.87-2.80 (m, 2H), 2.21-2.12 (m, 2H), 1.95-1.85 (m, 2H), 1.55-1.45 (m, 2H).

### Example 579: 4-((1-(4-(2-(2-Aminopyridin-3-yl)-5-(5-(difluoromethoxy)pyridin-2-yl)-3H-imidazo[4,5-b]pyridin-3-yl)benzyl)piperidin-4-yl)amino)pyrimidine-2-carbonitrile

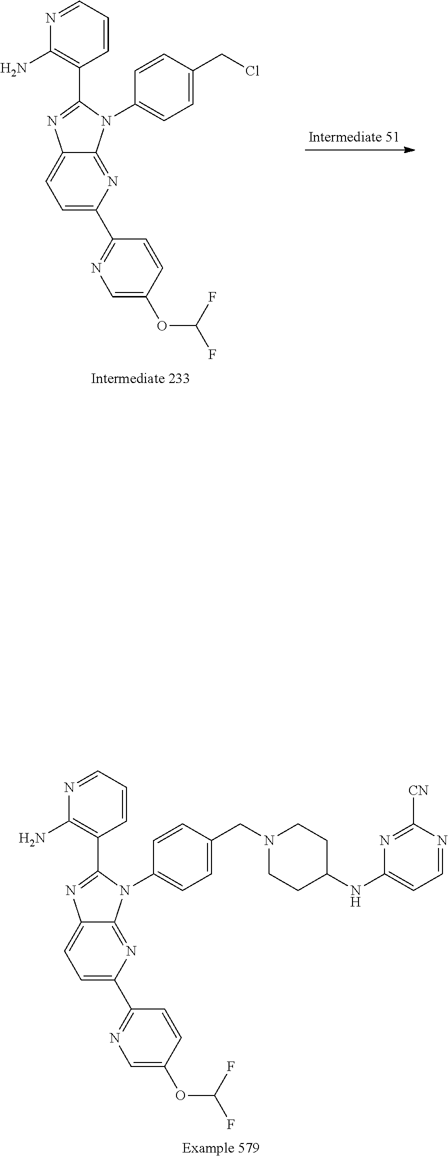 [SMILES: Nc1ncccc1-c1nc2ccc(-c3ccc(OC(F)F)cn3)nc2n1-c1ccc(CCl)cc1 ; N#Cc1nccc(NC2CCN(Cc3ccc(-n4c(-c5cccnc5N)nc5ccc(-c6ccc(OC(F)F)cn6)nc54)cc3)CC2)n1]

To a solution of Intermediate 233 (115 mg, 240 μmol, HCl salt) and Intermediate 51 (76.2 mg, 240 μmol, TFA) in DMF (2 mL) were added K₂CO₃ (166 mg, 1.20 mmol) and NaI (10.8 mg, 72 μmol). The mixture was stirred at 25° C. for 16 hr. The reaction mixture was poured into H₂O (15 mL) and extracted with EtOAc (15 mL×3). The organic phase was dried over anhydrous Na₂SO₄, filtered and concentrated under reduced pressure. After purified by silica gel flash chromatography (Eluent of 0%˜6% MeOH in CH₂Cl₂), 4-((1-(4-(2-(2-aminopyridin-3-yl)-5-(5-(difluoromethoxy)pyridin-2-yl)-3H-imidazo[4,5-b]pyridin-3-yl)benzyl)piperidin-4-yl)amino)pyrimidine-2-carbonitrile (Example 579, 54.5 mg, yield: 35% for three steps) was obtained as an off white solid. MS: m/z=646.3 [M+H]⁺. ¹H NMR (400 MHz, Dimethylsulfoxide-d₆) δ 8.57 (d, J=2.8 Hz, 1H), 8.41 (d, J=8.4 Hz, 1H), 8.41 (d, J=8.4 Hz, 1H), 8.23 (d, J=8.8 Hz, 1H), 8.12-8.03 (m, 2H), 8.03-7.98 (m, 1H), 7.77 (dd, J=8.8, 2.8 Hz, 1H), 7.57-7.52 (m, 0.5H), 7.51-7.46 (m, 3H), 7.39-7.31 (m, 0.5H), 7.19-7.12 (m, 1H), 7.09 (s, 2H), 6.78-6.60 (m, 1H), 6.39 (dd, J=7.6, 4.8 Hz, 1H), 3.93-3.74 (m, 1H), 3.60 (br s, 2H), 2.88-2.79 (m, 2H), 2.23-2.10 (m, 2H), 1.99-1.83 (m, 2H), 1.58-1.43 (m, 2H). ¹⁹F NMR (400 MHz, Dimethysulfoxide-d₆) δ −82.472.

### Example 580: 4-((1-(4-(2-(2-Aminopyridin-3-yl)-5-(5-(fluoromethoxy)pyridin-2-yl)-3H-imidazo[4,5-b]pyridin-3-yl)benzyl)piperidin-4-yl)amino)pyrimidine-2-carbonitrile

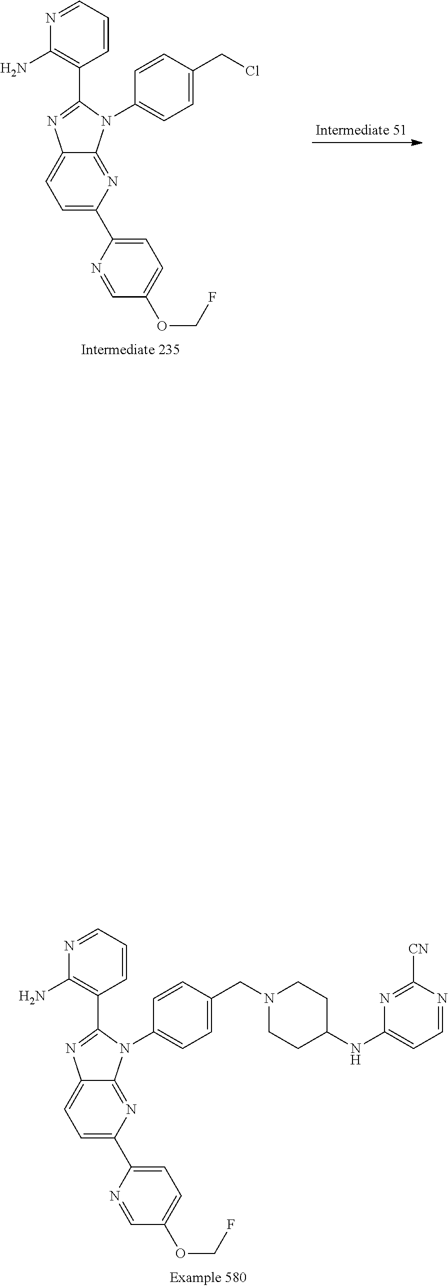 [SMILES: Nc1ncccc1-c1nc2ccc(-c3ccc(OCF)cn3)nc2n1-c1ccc(CCl)cc1 ; N#Cc1nccc(NC2CCN(Cc3ccc(-n4c(-c5cccnc5N)nc5ccc(-c6ccc(OCF)cn6)nc54)cc3)CC2)n1]

To a solution of Intermediate 235 (150 mg, 325.4 μmol) and Intermediate 51 (79 mg, 390 μmol) in DMF (3 mL) were added K₂CO₃ (180 mg, 1.3 mmol) and NaI (2.4 mg, 16 μmol). The mixture was stirred at 50° C. for 1 hr. The reaction mixture was quenched with H₂O (10 mL) at 25° C. and extracted with CH₂Cl₂ (15 mL×2). The combined organic layers were washed with brine (50 mL×3), dried over Na₂SO₄, filtered, and concentrated under reduced pressure. After purified by silica gel flash chromatography (Eluent of 0%˜8% MeOH in CH₂Cl₂), 4-((1-(4-(2-(2-aminopyridin-3-yl)-5-(5-(fluoromethoxy)pyridin-2-yl)-3H-imidazo[4,5-b]pyridin-3-yl)benzyl)piperidin-4-yl)amino)pyrimidine-2-carbonitrile (Example 580, 51.5 mg, yield: 25% for three steps) was obtained as a yellow solid. MS: m/z=628.2 [M+H]⁺. ¹H NMR (400 MHz, Dimethylsulfoxide-d₆) 8.51 (d, J=2.8 Hz, 1H), 8.39 (d, J=8.4 Hz, 1H), 8.30 (d, J=8.4 Hz, 1H), 8.19 (d, J=8.8 Hz, 1H), 8.25-8.01 (m, 2H), 8.00 (dd, J=4.8, 2.0 Hz, 1H), 7.68 (dd, J=8.8, 3.2 Hz, 1H), 7.51-7.45 (m, 4H), 7.17 (dd, J=7.6, 1.6 Hz, 1H), 7.01 (s, 2H), 6.68 (d, J=6.0 Hz, 1H), 6.38 (dd, J=7.6, 4.8 Hz, 1H), 5.96 (d, J=53.6 Hz, 2H), 3.87-3.76 (m, 1H), 3.60 (s, 2H), 2.90-2.80 (m, 2H), 2.24-2.10 (m, 2H), 1.95-1.85 (m, 2H), 1.57-1.44 (m, 2H). ¹⁹F NMR (400 MHz, Dimethylsulfoxide-d₆) δ −151.294.

### Example 581: 4-((1-(4-(2-(2-Aminopyridin-3-yl)-5-(5-methoxypyrimidin-2-yl)-3H-imidazo[4,5-b]pyridin-3-yl)benzyl)piperidin-4-yl)amino)pyrimidine-2-carbonitrile

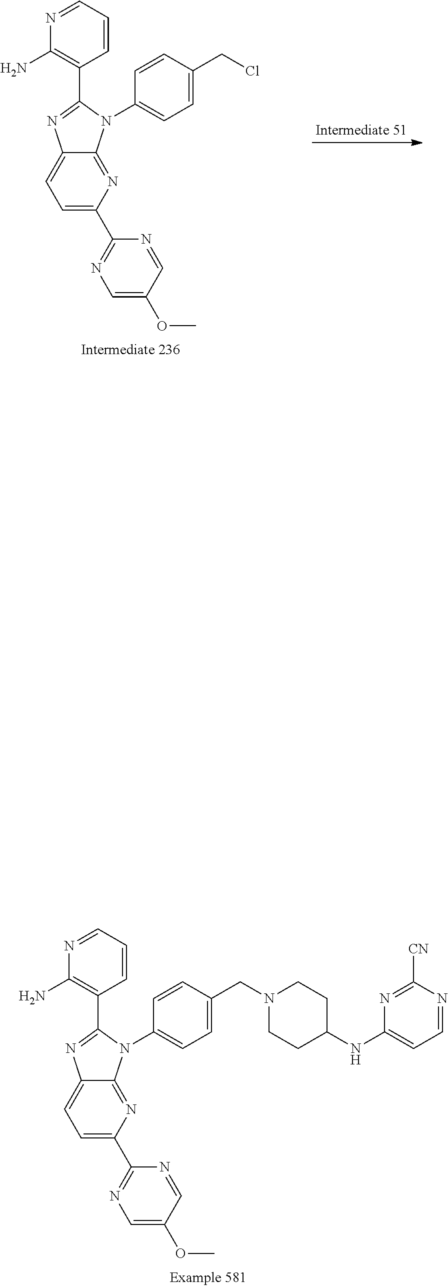 [SMILES: COc1cnc(-c2ccc3nc(-c4cccnc4N)n(-c4ccc(CCl)cc4)c3n2)nc1 ; COc1cnc(-c2ccc3nc(-c4cccnc4N)n(-c4ccc(CN5CCC(Nc6ccnc(C#N)n6)CC5)cc4)c3n2)nc1]

To a solution of Intermediate 236 (226 mg, 470 μmol, HCl salt) and Intermediate 51 (149 mg, 470 μmol, TFA) in DMF (4 mL) were added K₂CO₃ (325 mg, 2.35 mmol) and NaI (7.05 mg, 47.0 μmol). The mixture was stirred at 25° C. for 3 hr. The reaction mixture was quenched with H₂O (10 mL) at 25° C. and extracted with CH₂Cl₂ (15 mL×2). The combined organic layers were washed with brine (50 mL×3), dried over Na₂SO₄, filtered and concentrated under reduced pressure. The crude product was triturated with DMF (5 mL) at 25° C. for 3 minutes and MeCN (10 mL) at 25° C. for 10 minutes. The solid was collected, washed with MeCN (5 mL×2), dried under reduced pressure to give 4-((1-(4-(2-(2-aminopyridin-3-yl)-5-(5-methoxypyrimidin-2-yl)-3H-imidazo[4,5-b]pyridin-3-yl)benzyl)piperidin-4-yl)amino)pyrimidine-2-carbonitrile (Example 581, 83.6 mg, yield: 27% for two steps) as a yellow solid. MS: m/z=611.3 [M+H]⁺. ¹H NMR (400 MHz, Dimethylsulfoxide-d₆) δ 8.66 (s, 2H), 8.42-8.38 (m, 1H), 8.33-8.28 (m, 1H), 8.14 (d, J=7.2 Hz, 1H), 8.08 (d, J=6.0 Hz, 1H), 7.99 (dd, J=4.8, 1.6 Hz, 1H), 7.51-7.41 (m, 4H), 7.13 (dd, J=7.6, 1.6 Hz, 1H), 7.00 (br s, 2H), 6.70 (d, J=6.0 Hz, 1H), 6.37 (dd, J=7.6, 4.8 Hz, 1H), 3.96 (s, 3H), 3.88-3.73 (m, 1H), 3.59 (s, 2H), 2.86-2.79 (m, 2H), 2.22-2.12 (m, 2H), 1.94-1.85 (m, 2H), 1.57-1.46 (m, 2H).

### Example 582: 4-((1-(4-(2-(2-Aminopyridin-3-yl)-5-(5-methylpyrazin-2-yl)-3H-imidazo[4,5-b]pyridin-3-yl)benzyl)piperidin-4-yl)amino)pyrimidine-2-carbonitrile

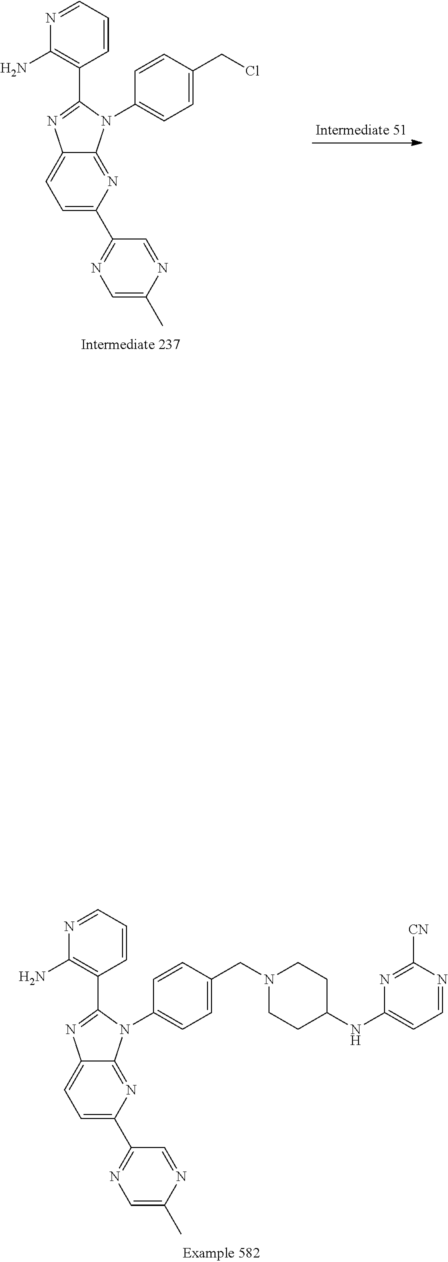 [SMILES: Cc1cnc(-c2ccc3nc(-c4cccnc4N)n(-c4ccc(CCl)cc4)c3n2)cn1 ; Cc1cnc(-c2ccc3nc(-c4cccnc4N)n(-c4ccc(CN5CCC(Nc6ccnc(C#N)n6)CC5)cc4)c3n2)cn1]

To a solution of Intermediate 237 (158 mg, 340 μmol, HCl salt) and Intermediate 51 (109 mg, 345 μmol, TFA) in DMF (5 mL) were added NaI (80.7 mg, 538 μmol) and K₂CO₃ (372 mg, 2.69 mmol). The mixture was stirred at 50° C. for 1 hr. The reaction mixture was diluted with H₂O (50 mL) and extracted with EtOAc (50 mL×3). The combined organic layers were washed with brine (100 mL), dried over Na₂SO₄, filtered and concentrated under reduced pressure. After purified by prep-HPLC (column: Waters xbridge 150×25 mm 10 μm; mobile phase: [water (NH₄HCO₃)-ACN]; gradient: 30%-60% B over 10 min), 4-((1-(4-(2-(2-aminopyridin-3-yl)-5-(5-methylpyrazin-2-yl)-3H-imidazo[4,5-b]pyridin-3-yl)benzyl)piperidin-4-yl)amino)pyrimidine-2-carbonitrile (Example 582, 106.9 mg, yield: 33%) was obtained as a yellow solid. MS: m/z=595.3 [M+H]⁺. ¹H NMR (400 MHz, Dimethylsulfoxide-d₆) δ 9.21 (s, 1H), 8.62 (s, 1H), 8.35-8.33 (m, 2H), 8.10-8.01 (m, 3H), 7.51-7.47 (m, 4H), 7.20 (dd, J=7.6, 1.6 Hz, 1H), 7.03 (s, 2H), 6.67 (d, J=6.4 Hz, 1H), 6.40 (dd, J=7.6, 4.8 Hz, 1H), 3.88-3.77 (m, 1H), 3.60 (s, 2H), 2.86-2.81 (m, 2H), 2.54 (s, 3H), 2.20-2.13 (m, 2H), 1.93-1.86 (m, 2H), 1.55-1.47 (m, 2H).

### Example 583: 4-((1-(4-(2-(2-Aminopyridin-3-yl)-5-(5-methoxypyrazin-2-yl)-3H-imidazo[4,5-b]pyridin-3-yl)benzyl)piperidin-4-yl)amino)pyrimidine-2-carbonitrile

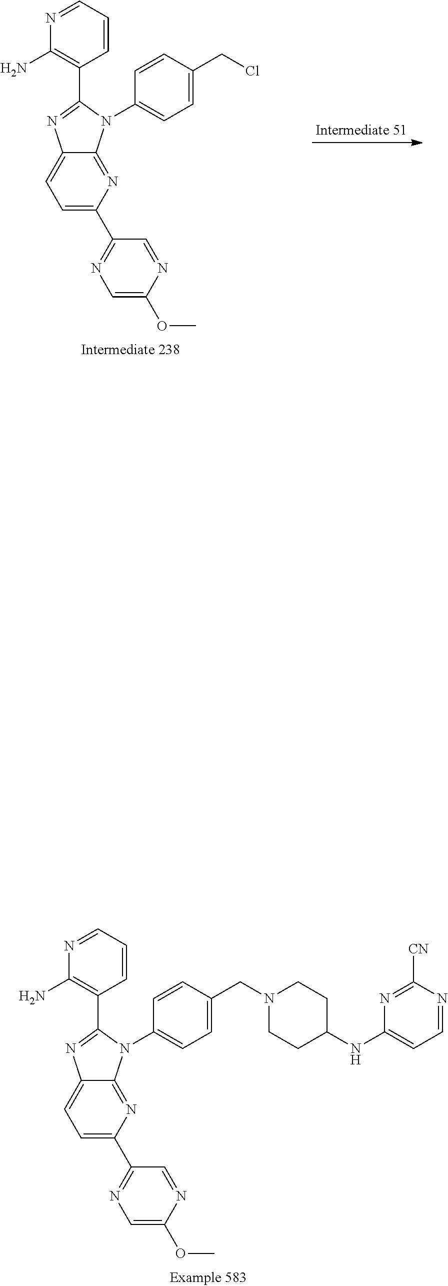 [SMILES: COc1cnc(-c2ccc3nc(-c4cccnc4N)n(-c4ccc(CCl)cc4)c3n2)cn1 ; COc1cnc(-c2ccc3nc(-c4cccnc4N)n(-c4ccc(CN5CCC(Nc6ccnc(C#N)n6)CC5)cc4)c3n2)cn1]

To a solution of Intermediate 238 (250 mg, 520 μmol, HCl salt) and Intermediate 51 (106 mg, 333 μmol, TFA) in DMF (5 mL) were added NaI (78 mg, 520 μmol) and K₂CO₃ (360 mg, 2.60 mmol). The mixture was stirred at 50° C. for 1 hr. The reaction mixture was diluted with H₂O (50 mL) and extracted with EtOAc (50 mL×3). The combined organic layers were washed with brine (100 mL), dried over Na₂SO₄, filtered and concentrated under reduced pressure. After purified by prep-HPLC (column: Waters xbridge 150×25 mm 10 μm; mobile phase: [water (NH₄HCO₃)-ACN]; gradient: 36%-66% B over 10 min), 4-((1-(4-(2-(2-aminopyridin-3-yl)-5-(5-methoxypyrazin-2-yl)-3H-imidazo[4,5-h]pyridin-3-yl)benzyl)piperidin-4-yl)amino)pyrimidine-2-carbonitrile (Example 583, 110.2 mg, yield: 34%) was obtained as a yellow solid. MS: m/z=611.2 [M+H]⁺. ¹H NMR (400 MHz, Dimethylsulfoxide-d₆) δ 8.91 (d, J=1.2 Hz, 1H), 8.40 (d, J=1.2 Hz, 1H), 8.32 (d, J=8.4 Hz, 1H), 8.25 (d, J=8.4 Hz, 1H), 8.10-8.00 (m, 3H), 7.48 (dd, J=14.0, 8.4 Hz, 4H), 7.20 (dd, J=7.2, 1.6 Hz, 1H), 7.03 (s, 2H), 6.67 (d, J=6.0 Hz, 1H), 6.40 (dd, J=7.6, 4.8 Hz, 1H), 3.95 (s, 3H), 3.83-3.81 (m, 1H), 3.60 (s, 2H), 2.86-2.81 (m, 2H), 2.22-2.14 (m, 2H), 1.94-1.86 (m, 2H), 1.55-1.47 (m, 2H)

### Example 584: 4-((1-(4-(2-(2-Aminopyridin-3-yl)-5-(6-ethoxypyridin-3-yl)-3H-imidazo[4,5-b]pyridin-3-yl)benzyl)piperidin-4-yl)amino)pyrimidine-2-carbonitrile

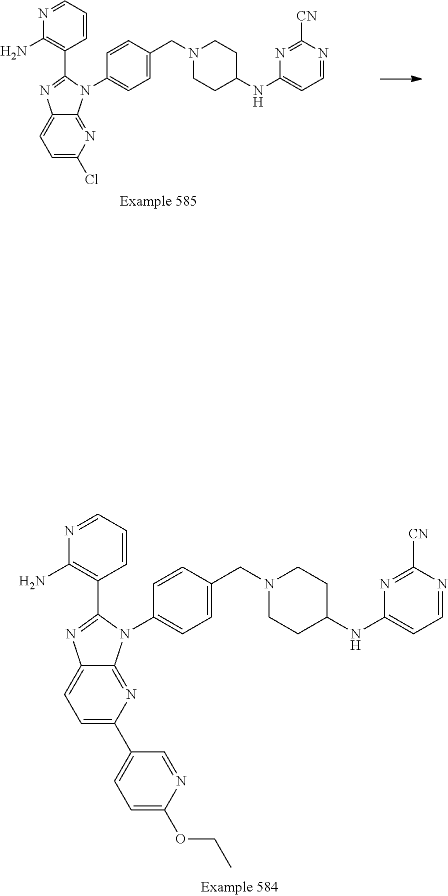 [SMILES: N#Cc1nccc(NC2CCN(Cc3ccc(-n4c(-c5cccnc5N)nc5ccc(Cl)nc54)cc3)CC2)n1 ; CCOc1ccc(-c2ccc3nc(-c4cccnc4N)n(-c4ccc(CN5CCC(Nc6ccnc(C#N)n6)CC5)cc4)c3n2)cn1]

To a mixture of Example 405 (300 mg, 559 μmol) and (6-ethoxypyridin-3-yl)boronic acid (103 mg, 615 μmol) in 1,4-dioxane (2 mL) and H₂O (0.5 mL) were added Cs₂CO₃ (546 mg, 1.68 mmol) and Pd(dppf)Cl₂ (40.9 mg, 55.9 μmol). The mixture was stirred at 100° C. for 2 hr under N₂. The mixture was diluted with H₂O (50 mL) and extracted with EtOAc (100 mL×2). The combined organic layers were washed with brine (100 mL), dried over anhydrous Na₂SO₄, filtered and concentrated. After purified by prep-HPLC (column: Phenomenex C18 80×30 mm×5 μm; mobile phase: [water (NH₃H₂O+NH₄HCO₃)-ACN]; gradient: 55%-85% B over 7 min), 4-((1-(4-(2-(2-aminopyridin-3-yl)-5-(6-ethoxypyridin-3-yl)-3H-imidazo[4,5-b]pyridin-3-yl)benzyl)piperidin-4-yl)amino)pyrimidine-2-carbonitrile (Example 584, 119.7 mg, yield: 34%) was obtained as a yellow solid. MS: m/z=624.1 [M+H]⁺. ¹H NMR (400 MHz, Dimethylsulfoxide-d₆) δ 8.83 (d, J=2.0 Hz, 1H), 8.34-8.22 (m, 2H), 8.13-7.94 (m, 4H), 7.51-7.42 (m, 4H), 7.15 (d, J=7.2 Hz, 1H), 7.04 (s, 2H), 6.88 (d, J=8.8 Hz, 1H), 6.73-6.63 (m, 1H), 6.38 (dd, J=8.0, 5.2 Hz, 1H), 4.34 (m, J=7.2 Hz, 2H), 3.87-3.76 (m, 1H), 3.59 (s, 2H), 2.92-2.74 (m, 2H), 2.22-2.09 (m, 2H), 1.93-1.78 (m, 2H), 1.56-1.44 (m, 2H), 1.33 (t, J=7.2 Hz, 3H).

### Example 585: 4-((1-(4-(2-(2-Aminopyridin-3-yl)-5-(5-cyclopropoxypyridin-2-yl)-3H-imidazo[4,5-b]pyridin-3-yl)benzyl)piperidin-4-yl)amino)pyrimidine-2-carbonitrile

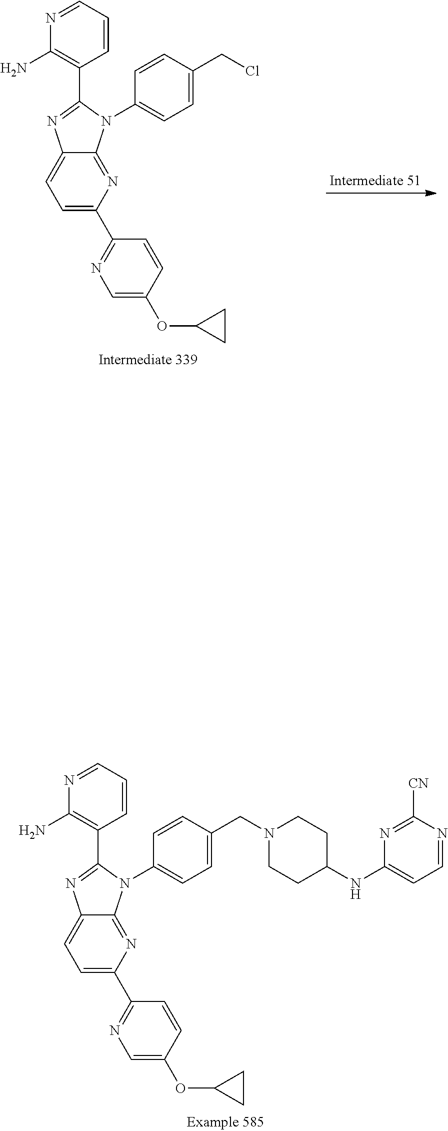 [SMILES: Nc1ncccc1-c1nc2ccc(-c3ccc(OC4CC4)cn3)nc2n1-c1ccc(CCl)cc1 ; N#Cc1nccc(NC2CCN(Cc3ccc(-n4c(-c5cccnc5N)nc5ccc(-c6ccc(OC7CC7)cn6)nc54)cc3)CC2)n1]

To a solution of Intermediate 339 (166 mg, 354 μmol, HCl salt) and Intermediate 51 (108 mg, 531 μmol) in DMF (1 mL) were added K₂CO₃ (147 mg, 1.06 mmol) and NaI (5.31 mg, 35.4 μmol). The mixture was stirred at 80° C. for 8 hr. The mixture was diluted with H₂O (10 mL) and extracted with ethyl acetate (10 mL×2). The combined organic layers were dried over anhydrous Na₂SO₄, filtered and concentrated. After purified by prep-HPLC (column: C18 150×30 mm; mobile phase: [water (NH₃H₂O+NH₄HCO₃)-ACN]; gradient: 54%-84% B over 7 min), 4-((1-(4-(2-(2-aminopyridin-3-yl)-5-(5-cyclopropoxypyridin-2-yl)-3H-imidazo[4,5-b]pyridin-3-yl)benzyl)piperidin-4-yl)amino)pyrimidine-2-carbonitrile (Example 585, 45.5 mg, yield: 20% for three steps) was obtained as a light yellow solid. MS: m/z=658.2 [M+Na]⁺. ¹H NMR (400 MHz, Dimethylsulfoxide-d₆) δ 8.45 (d, J=3.2 Hz, 1H), 8.36 (d, J=8.4 Hz, 1H), 8.27 (d, J=8.4 Hz, 1H), 8.14 (d, J=8.8 Hz, 1H), 8.13-8.02 (m, 2H), 8.00 (dd, J=4.8, 2.0 Hz, 1H), 7.60 (dd, J=8.4, 2.8 Hz, 1H), 7.51-7.44 (m, 4H), 7.16 (dd, J=8.0, 2.0 Hz, 1H), 7.02 (br s, 2H), 6.67 (d, J=6.0 Hz, 1H), 6.38 (dd, J=7.6, 4.8 Hz, 1H), 4.01-3.96 (m, 1H), 3.89-3.73 (m, 1H), 3.60 (s, 2H), 2.92-2.79 (m, 2H), 2.21-2.12 (m, 2H), 1.95-1.82 (m, 2H), 1.56-1.44 (m, 2H), 0.85-0.80 (m, 2H), 0.73-0.69 (m, 2H).

### Example 586: 4-((1-(4-(2-(2-Aminopyridin-3-yl)-5-(5-isopropoxypyridin-2-yl)-3H-imidazo[4,5-b]pyridin-3-yl)benzyl)piperidin-4-yl)amino)pyrimidine-2-carbonitrile

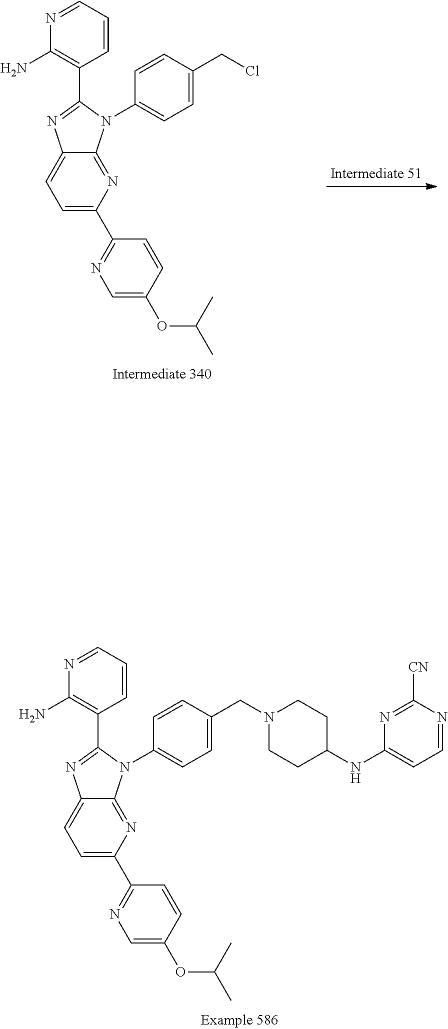 [SMILES: CC(C)Oc1ccc(-c2ccc3nc(-c4cccnc4N)n(-c4ccc(CCl)cc4)c3n2)nc1 ; CC(C)Oc1ccc(-c2ccc3nc(-c4cccnc4N)n(-c4ccc(CN5CCC(Nc6ccnc(C#N)n6)CC5)cc4)c3n2)nc1]

To a solution of Intermediate 340 (199 mg, 424 μmol) and Intermediate 51 (112 mg, 551 μmol) in DMF (1 mL) were added NaI (6.35 mg, 42.4 μmol) and K₂CO₃ (176 mg, 1.27 mmol). The mixture was stirred at 25° C. for 12 hr. The mixture was diluted with and extracted with EtOAc (5 mL×2). The combined organic layers were dried over anhydrous Na₂SO₄, filtered and concentrated. After purified by prep-HPLC (column: C18 150×30 mm; mobile phase: [water (NH₃H₂O+NH₄HCO₃)-ACN]; gradient: 59%-89% B over 7 min), 4-((1-(4-(2-(2-aminopyridin-3-yl)-5-(5-isopropoxypyridin-2-yl)-3H-imidazo[4,5-b]pyridin-3-yl)benzyl)piperidin-4-yl)amino)pyrimidine-2-carbonitrile (Example 586, 79.5 mg, yield: 29.4% for three steps) was obtained as a light yellow solid. MS: m/z=638.4 [M+H]⁺. ¹H NMR (400 MHz, Dimethylsulfoxide-d₆) δ 8.39-8.30 (m, 2H), 8.25 (d, J=8.4 Hz, 1H), 8.14-8.01 (m, 3H), 8.00 (dd, J=4.8, 1.6 Hz, 1H), 7.51-7.43 (m, 5H), 7.15 (dd, J=7.6, 1.2 Hz, 1H), 7.03 (br s, 2H), 6.68 (br d, J=6.4 Hz, 1H), 6.37 (dd, J=7.6, 4.8 Hz, 1H), 4.77-4.68 (m, 1H), 3.82-3.81 (m, 1H), 3.60 (s, 2H), 2.84-2.82 (m, 2H), 2.19-2.13 (m, 2H), 1.95-1.83 (m, 2H), 1.55-1.42 (m, 2H), 1.30 (d, J=6.0 Hz, 6H).

### Example 587: 4-((1-(4-(2-(2-Aminopyridin-3-yl)-5-(6-cyclopropylpyridin-3-yl)-3H-imidazo[4,5-b]pyridin-3-yl)benzyl)piperidin-4-yl)amino)pyrimidine-2-carbonitrile

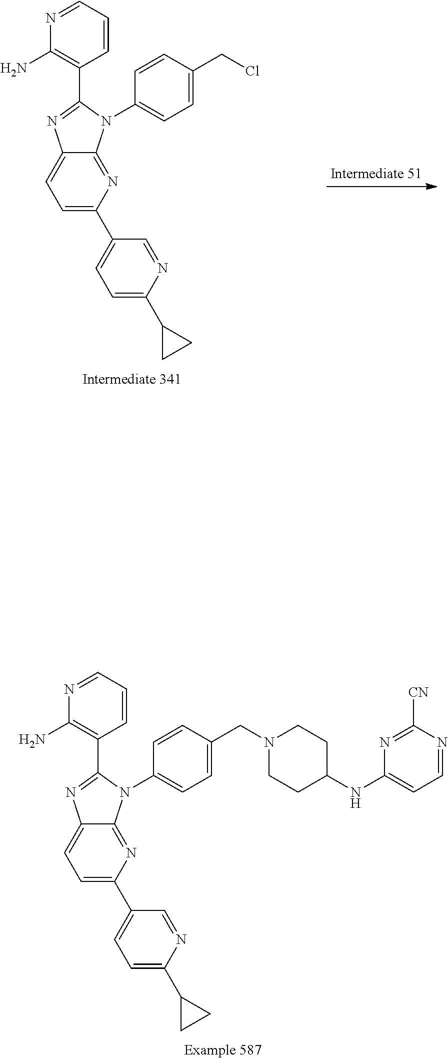 [SMILES: Nc1ncccc1-c1nc2ccc(-c3ccc(C4CC4)nc3)nc2n1-c1ccc(CCl)cc1 ; N#Cc1nccc(NC2CCN(Cc3ccc(-n4c(-c5cccnc5N)nc5ccc(-c6ccc(C7CC7)nc6)nc54)cc3)CC2)n1]

To a solution of Intermediate 341 (190 mg, 420 μmol, HCl salt) and Intermediate 51 (102 mg, 503 mol) in DMF (3 mL) were added K₂CO₃ (174 mg, 1.26 mmol) and NaI (6.29 mg, 42.0 μmol). The mixture was stirred at 25° C. for 12 hr. The mixture was diluted with H₂O (10 mL) and extracted with ethyl acetate (10 mL×2). The combined organic layers were dried over anhydrous Na₂SO₄, filtered and concentrated. After purified by prep-HPLC (column: C18 150×30 mm; mobile phase: [water (NH₃H₂O+NH₄HCO₃)-ACN]; gradient: 45%-75% B over 7 min), 4-((1-(4-(2-(2-aminopyridin-3-yl)-5-(6-cyclopropylpyridin-3-yl)-3H-imidazo[4,5-b]pyridin-3-yl)benzyl)piperidin-4-yl)amino)pyrimidine-2-carbonitrile (Example 587, 93.4 mg, yield: 36% for three steps) was obtained as a light yellow solid. MS: m/z=620.3 [M+H]⁺. ¹H NMR (400 MHz, Dimethylsulfoxide-d₆) δ 9.05 (d, J=2.4 Hz, 1H), 8.28 (d, J=8.4 Hz, 1H), 8.21 (dd, J=8.4, 2.4 Hz, 1H), 8.07 (dd, J=12.8, 6.0 Hz, 2H), 8.02-7.97 (m, 2H), 7.50-7.42 (m, 4H), 7.37 (d, J=8.4 Hz, 1H), 7.16 (dd, J=7.6, 1.6 Hz, 1H), 7.04 (br s, 2H), 6.67 (d, J=6.4 Hz, 1H), 6.38 (dd, J=7.6, 4.8 Hz, 1H), 3.95-3.71 (m, 1H), 3.59 (s, 2H), 2.90-2.75 (m, 2H), 2.21-2.09 (m, 3H), 1.99-1.82 (m, 2H), 1.59-1.45 (m, 2H), 0.99-0.93 (m, 4H).

### Example 588: 4-((1-(4-(2-(2-Aminopyridin-3-yl)-5-(3-methyl-4H-1,2,4-triazol-4-yl)-3H-imidazo[4,5-b]pyridin-3-yl)benzyl)piperidin-4-yl)amino)pyrimidine-2-carbonitrile

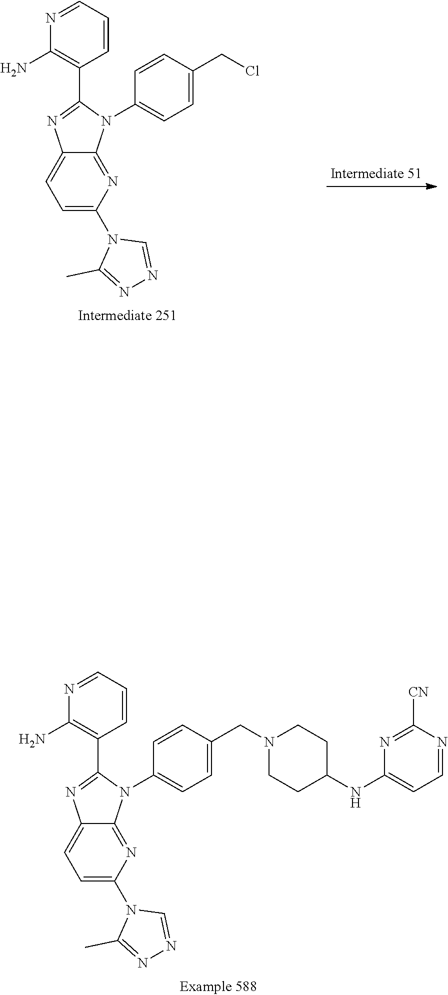 [SMILES: Cc1nncn1-c1ccc2nc(-c3cccnc3N)n(-c3ccc(CCl)cc3)c2n1 ; Cc1nncn1-c1ccc2nc(-c3cccnc3N)n(-c3ccc(CN4CCC(Nc5ccnc(C#N)n5)CC4)cc3)c2n1]

To a solution of Intermediate 251 (166 mg, 366 μmol, HCl salt) and Intermediate 51 (213 mg, 671 μmol, TFA salt) in DMF (2 mL) were added K₂CO₃ (253 mg, 1.83 mmol) and NaI (16.5 mg, 110 μmol). The mixture was stirred at 25° C. for 1 hr. The reaction mixture was poured into H₂O (20 mL) and extracted with EtOAc (20 mL×3). The combined organic phases were dried over anhydrous Na₂SO₄, filtered and concentrated under reduced pressure. After purified by silica gel flash chromatography (Eluent of 0%˜5% MeOH in CH₂Cl₂), 4-((1-(4-(2-(2-aminopyridin-3-yl)-5-(3-methyl-4H-1,2,4-triazol-4-yl)-3H-imidazo[4,5-b]pyridin-3-yl)benzyl)piperidin-4-yl)amino)pyrimidine-2-carbonitrile (Example 588, 71.4 mg, yield: 33% for three steps) was obtained as a yellow solid. MS: m/z=584.4 [M+H]^{+ 1}H NMR (400 MHz, Dimethylsulfoxide-d₆) δ 8.43 (d, J=8.4 Hz, 1H), 8.19-7.91 (m, 4H), 7.83 (d, J=8.4 Hz, 1H), 7.52-7.41 (m, 4H), 7.25 (dd, J=7.6, 1.6 Hz, 1H), 7.04 (s, 2H), 6.74-6.60 (m, 1H), 6.41 (dd, J=7.6, 4.8 Hz, 1H), 3.91-3.69 (m, 1H), 3.58 (br s, 2H), 2.83-2.75 (m, 2H), 2.56 (s, 3H), 2.19-2.07 (m, 2H), 1.96-1.80 (m, 2H), 1.54-1.42 (m, 2H).

### Example 589: 4-((1-(4-(2-(2-Aminopyridin-3-yl)-5-(3-methyl-1H-1,2,4-triazol-1-yl)-3H-imidazo[4,5-b]pyridin-3-yl)benzyl)piperidin-4-yl)amino)pyrimidine-2-carbonitrile

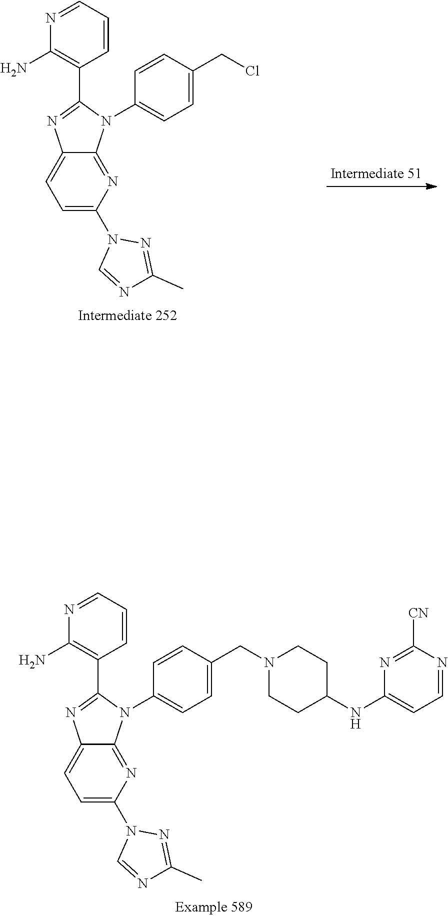 [SMILES: Cc1ncn(-c2ccc3nc(-c4cccnc4N)n(-c4ccc(CCl)cc4)c3n2)n1 ; Cc1ncn(-c2ccc3nc(-c4cccnc4N)n(-c4ccc(CN5CCC(Nc6ccnc(C#N)n6)CC5)cc4)c3n2)n1]

To a solution of Intermediate 252 (150 mg, 360 μmol, HCl salt) and Intermediate 51 (73.1 mg, 360 μmol, TFA) in DMF (2 mL) were added K₂CO₃ (149 mg, 1.08 mmol) and NaI (10.8 mg, 72.0 μmol). The mixture was stirred at 50° C. for 0.5 hr. The reaction mixture was quenched with H₂O (50 mL) at 25° C., diluted with CH₂Cl₂ (50 mL), and extracted with CH₂Cl₂ (50 mL×2). The combined organic layers were washed with brine (30 mL×3), dried over Na₂SO₄, filtered and concentrated under reduced pressure. After purified by prep-HPLC (column: Daisogel SP ODS RPS 150×25 mm×5 μm; mobile phase: [water (NH₄HCO₃)-ACN]; gradient: 30%-60% B over 10 min), 4-((1-(4-(2-(2-aminopyridin-3-yl)-5-(3-methyl-1H-1,2,4-triazol-1-yl)-3H-imidazo[4,5-b]pyridin-3-yl)benzyl)piperidin-4-yl)amino)pyrimidine-2-carbonitrile (Example 589, 59.1 mg, yield: 26% for three steps) was obtained as a light yellow solid. MS: m/z=584.3 [M+H]⁺. ¹H NMR (400 MHz, Dimethylsulfoxide-d₆) δ 8.97 (s, 1H), 8.41 (d, J=8.4 Hz, 1H), 8.10-8.05 (m, 2H), 8.02-7.99 (m, 1H), 7.85-7.84 (m, 1H), 7.48-7.42 (m, 4H), 7.17 (dd, J=7.6, 1.2 Hz, 1H), 6.93 (s, 2H), 6.67 (d, J=6.0 Hz, 1H), 6.40 (dd, J=7.6, 4.8 Hz, 1H), 3.86-3.78 (m, 1H), 3.58 (s, 2H), 2.85-2.80 (m, 2H), 2.38 (s, 3H), 2.18-2.12 (m, 2H), 1.93-1.86 (m, 2H), 1.54-1.47 (m, 2H).

### Example 590: 4-((1-(4-(2-(2-Aminopyridin-3-yl)-5-(1H-1,2,4-triazol-1-yl)-3H-imidazo[4,5-b]pyridin-3-yl)benzyl)piperidin-4-yl)amino)pyrimidine-2-carbonitrile

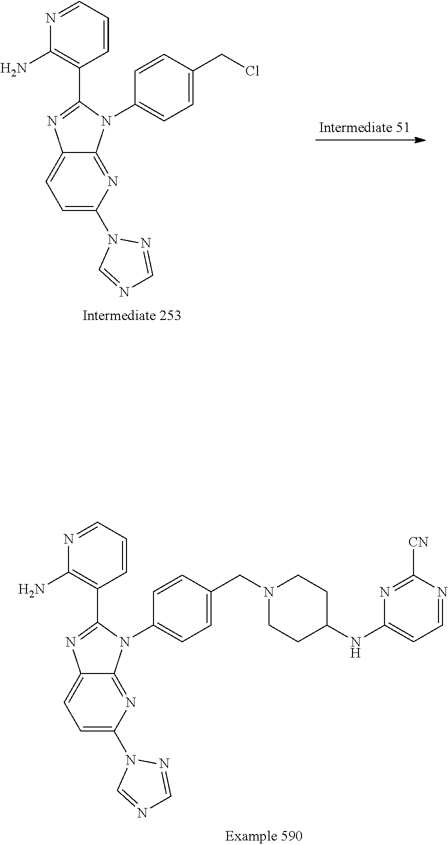 [SMILES: Nc1ncccc1-c1nc2ccc(-n3cncn3)nc2n1-c1ccc(CCl)cc1 ; N#Cc1nccc(NC2CCN(Cc3ccc(-n4c(-c5cccnc5N)nc5ccc(-n6cncn6)nc54)cc3)CC2)n1]

To a solution of Intermediate 253 (150 mg, 372 μmol, HCl) and Intermediate 51 (75.7 mg, 372 μmol, TFA) in DMF (3 mL) were added K₂CO₃ (154 mg, 1.12 mmol) and NaI (11.2 mg, 74.5 μmol). The mixture was stirred at 50° C. for 0.5 hr. The reaction mixture was quenched with H₂O (50 mL) at 25° C., diluted with CH₂Cl₂ (5 mL), and extracted with CH₂Cl₂ (50 mL×2). The combined organic layers were washed with brine (30 mL×3), dried over Na₂SO₄, filtered and concentrated under reduced pressure. After purified by prep-HPLC (column: Phenomenex C18 150×25 mm×10 μm; mobile phase: [water (NH₃H₂O)-ACN]; gradient: 38%-68%, over 11 min), 4-((1-(4-(2-(2-aminopyridin-3-yl)-5-(1H-1,2,4-triazol-1-yl)-3H-imidazo[4,5-b]pyridin-3-yl)benzyl)piperidin-4-yl)amino)pyrimidine-2-carbonitrile (Example 590, 59.1 mg, yield: 38% for three steps) was obtained as a light yellow solid. MS: m/z=570.2 [M+H]⁺. ¹H NMR (400 MHz, Dimethylsulfoxide-d₆) δ 9.12 (s, 1H), 8.45 (d, J=8.4 Hz, 1H), 8.29 (s, 1H), 8.10-8.04 (m, 2H), 8.02-8.00 (m, 1H), 7.89 (d, J=8.8 Hz, 1H), 7.46-7.44 (m, 4H), 7.20-7.16 (m, 1H), 6.93 (s, 2H), 6.67 (d, J=5.6 Hz, 1H), 6.40 (dd, J=7.6, 4.8 Hz, 1H), 3.88-3.76 (m, 1H), 3.58 (s, 2H), 2.85-2.81 (m, 2H), 2.18-2.12 (m, 2H), 1.89-1.87 (m, 2H), 1.54-1.47 (m, 2H).

### Example 591: 4-((1-(4-(2-(2-Aminopyridin-3-yl)-5-(1H-1,2,3-triazol-1-yl)-3H-imidazo[4,5-b]pyridin-3-yl)benzyl)piperidin-4-yl)amino)pyrimidine-2-carbonitrile

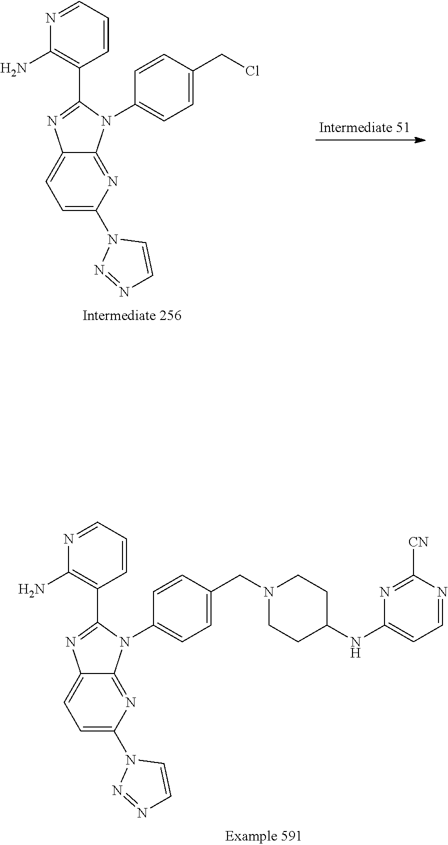 [SMILES: Nc1ncccc1-c1nc2ccc(-n3ccnn3)nc2n1-c1ccc(CCl)cc1 ; N#Cc1nccc(NC2CCN(Cc3ccc(-n4c(-c5cccnc5N)nc5ccc(-n6ccnn6)nc54)cc3)CC2)n1]

To a solution of Intermediate 256 (48.2 mg, 110 μmol, HCl) and Intermediate 51 (34.8 mg, 110 μmol, TFA) in DMF (1 mL) were added K₂CO₃ (45.5 mg, 329 μmol) and NaI (16.4 mg, 110 μmol). The mixture was degassed and purged with N₂ three times and stirred at 25° C. for 16 hr under N₂. The reaction mixture was diluted with CH₂Cl₂ (10 mL) and washed with brine (10 mL×3), dried over Na₂SO₄, filtered and concentrated under reduced pressure. After purified byprep-TLC (SiO₂, CH₂Cl₂:MeOH=10:1), 4-((1-(4-(2-(2-aminopyridin-3-yl)-5-(1H-1,2,3-triazol-1-yl)-3H-imidazo[4,5-b]pyridin-3-yl)benzyl)piperidin-4-yl)amino)pyrimidine-2-carbonitrile (Example 591, 8.6 mg, yield 13% 13% for three steps) was obtained as a yellow solid. MS: m/z=570.1 [M+H]⁺. ¹H NMR (400 MHz, Methanol-d₄) δ 8.59 (s, 1H), 8.41 (d, J=8.8 Hz, 1H), 8.24 (d, J=8.4 Hz, 1H), 8.05-7.96 (m, 2H), 7.86 (s, 1H), 7.57 (d, J=8.4 Hz, 2H), 7.46 (d, J=8.4 Hz, 2H), 7.33 (dd, J=8.0, 2.0 Hz, 1H), 6.64-6.57 (m, 1H), 6.48 (dd, J=7.6, 5.2 Hz, 1H), 4.06-3.89 (m, 1H), 3.70 (s, 2H), 3.04-2.94 (m, 2H), 2.37-2.26 (m, 2H), 2.07-1.98 (m, 2H), 1.66-1.59 (m, 2H).

### Example 592: 4-((1-(4-(2-(2-Aminopyridin-3-yl)-5-(2H-1,2,3-triazol-2-yl)-3H-imidazo[4,5-b]pyridin-3-yl)benzyl)piperidin-4-yl)amino)pyrimidine-2-carbonitrile

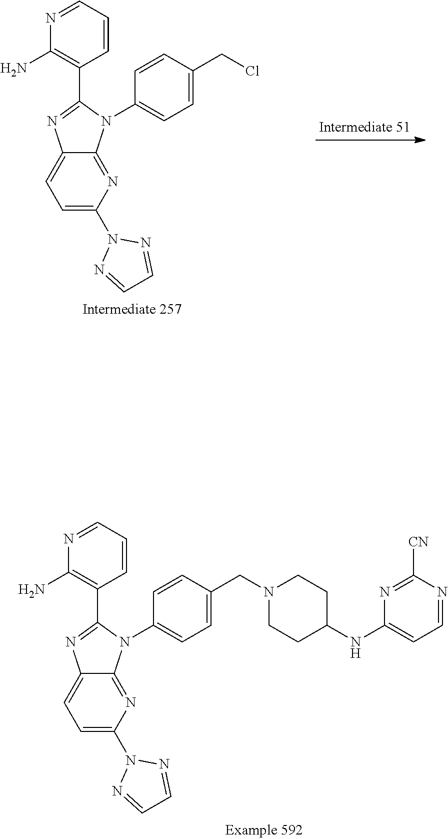 [SMILES: Nc1ncccc1-c1nc2ccc(-n3nccn3)nc2n1-c1ccc(CCl)cc1 ; N#Cc1nccc(NC2CCN(Cc3ccc(-n4c(-c5cccnc5N)nc5ccc(-n6nccn6)nc54)cc3)CC2)n1]

To a solution of Intermediate 257 (103 mg, 235 μmol, HCl) and Intermediate 51 (74.4 mg, 235 μmol, TFA) in DMF (I mL) were added K₂CO₃ (97.2 mg, 704 μmol) and NaI (35.2 mg, 235 μmol). The mixture was degassed and purged with N₂ for 3 times and stirred at 25° C. for 16 hr under N₂. The reaction mixture was diluted with CH₂Cl₂ (10 mL) and washed with brine (10 mL×3), dried over Na₂SO₄, filtered and concentrated under reduced pressure. After purified by silica gel flash chromatography (MeOH in CH₂Cl₂=1% to 7%), 4-((1-(4-(2-(2-aminopyridin-3-yl)-5-(2H-1,2,3-triazol-2-yl)-3H-imidazo[4,5-b]pyridin-3-yl)benzyl)piperidin-4-yl)amino)pyrimidine-2-carbonitrile (Example 592, 27.3 mg, yield 18% for three steps) was obtained as a pink solid. MS: m/z=570.2 [M+H]⁺. ¹H NMR (400 MHz, Dimethylsulfoxide-d₆) δ 8.44 (d, J=8.4 Hz, 1H), 8.13 (s, 2H), 8.11-8.02 (m, 3H), 8.00 (dd, J=4.8, 1.6 Hz, 1H), 7.50-7.42 (m, 4H), 7.19-7.15 (m, 1H), 6.95 (br s, 2H), 6.68 (d, J=5.6 Hz, 1H), 6.38 (dd, J=7.6, 4.8 Hz, 1H), 3.88-3.76 (m, 1H), 3.59 (s, 2H), 2.86-2.79 (m, 2H), 2.21-2.12 (m, 2H), 1.93-1.84 (m, 2H), 1.54-1.45 (m, 2H).

### Example 593: 4-((1-(4-(2-(2-Aminopyridin-3-yl)-5-(4-methyl-1H-1,2,3-triazol-1-yl)-3H-imidazo[4,5-b]pyridin-3-yl)benzyl)pyridin-4-yl amino)pyrimidine-2-carbonitrile

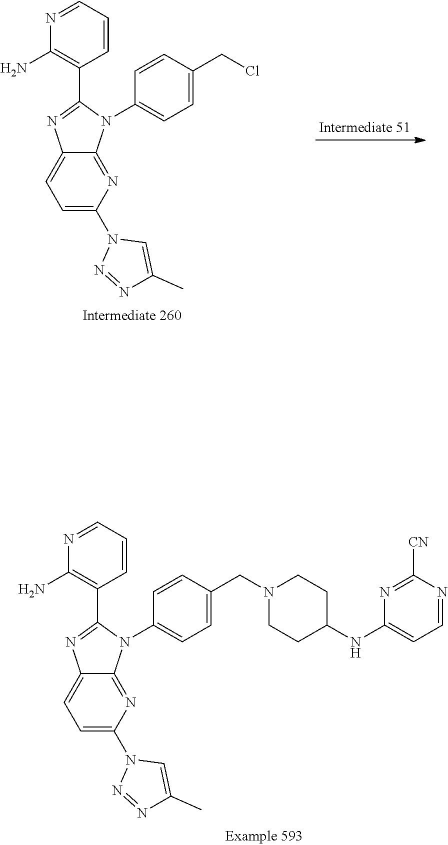 [SMILES: Cc1cn(-c2ccc3nc(-c4cccnc4N)n(-c4ccc(CCl)cc4)c3n2)nn1 ; Cc1cn(-c2ccc3nc(-c4cccnc4N)n(-c4ccc(CN5CCC(Nc6ccnc(C#N)n6)CC5)cc4)c3n2)nn1]

To a solution of Intermediate 260 (146 mg, 322 μmol, HCl salt), Intermediate 51 (175 mg, 552 μmol, TFA salt) in DMF (2 mL) were added K₂CO₃ (223 mg, 1.61 mmol) and NaI (14.5 mg, 96.6 μmol). The mixture was stirred at 25° C. for 16 hr. The reaction mixture was poured into H₂O (20 mL), extracted with EtOAc (20 mL×3). The organic phase was dried over anhydrous Na₂SO₄, filtered and concentrated under reduced pressure. After purified by silica gel flash chromatography (Eluent of 0%˜7% MeOH in CH₂Cl₂), 4-((1-(4-(2-(2-aminopyridin-3-yl)-5-(4-methyl-1H-1,2,3-triazol-1-yl)-3H-imidazo[4,5-b]pyridin-3-yl)benzyl)piperidin-4-yl)amino)pyrimidine-2-carbonitrile (Example 593, 18.3 mg, yield: 10% for three steps) was obtained as a yellow solid. MS: m/z=584.4 [M+H]^{+ 1}H NMR (400 MHz, Dimethylsulfoxide-d₆) δ 8.55-8.41 (m, 1H), 8.39-8.29 (m, 1H), 8.17-7.96 (m, 4H), 7.54-7.42 (m, 4H), 7.26-7.12 (m, 1H), 6.92 (br s, 2H), 6.75-6.60 (m, 1H), 6.48-6.34 (m, 1H), 3.91-3.74 (m, 1H), 3.58 (s, 2H), 2.89-2.77 (m, 2H), 2.32 (s, 3H), 2.23-2.10 (m, 2H), 1.97-1.81 (m, 2H), 1.60-1.44 (m, 2H).

### Example 594: 4-((1-(4-(2-(2-Aminopyridin-3-yl)-5-(oxazol-4-yl)-3H-imidazo[4,5-b]pyridin-3-yl)benzyl)piperidin-4-yl)amino)pyrimidine-2-carbonitrile

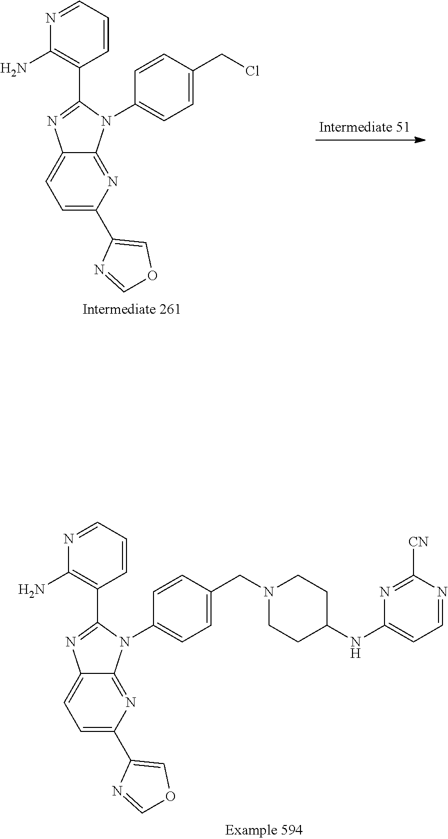 [SMILES: Nc1ncccc1-c1nc2ccc(-c3cocn3)nc2n1-c1ccc(CCl)cc1 ; N#Cc1nccc(NC2CCN(Cc3ccc(-n4c(-c5cccnc5N)nc5ccc(-c6cocn6)nc54)cc3)CC2)n1]

To a solution of Intermediate 261 (122 mg, 303 μmol, HCl salt) and Intermediate 51 (96 mg, 303 μmol, TFA) in DMF (3 mL) were added K₂CO₃ (167 mg, 1.21 mmol) and NaI (22.7 mg, 151 μmol). The mixture was stirred at 25° C. for 16 hr. The reaction mixture was quenched with H₂O (5 mL) at 25° C. and extracted with EtOAc (10 mL×3). The combined organic layers were washed with brine (50 mL×3), dried over Na₂SO₄, filtered and concentrated under reduced pressure. After purified by prep-HPLC (column: Phenomenex C18 150×25 mm×10 μm; mobile phase: [water (NH₄HCO₃)-ACN]; gradient: 26%-56% B over 11 min), 4-((1-(4-(2-(2-aminopyridin-3-yl)-5-(oxazol-4-yl)-3H-imidazo[4,5-b]pyridin-3-yl)benzyl)piperidin-4-yl)amino)pyrimidine-2-carbonitrile (Example 594, 62.6 mg, yield: 35% for three steps) was obtained as a yellow solid. MS: m/z=570.2 [M+H]⁺. ¹H NMR (400 MHz, Dimethylsulfoxide-d₆) δ 8.49 (s, 1H), 8.45 (s, 1H), 8.27 (d, J=8.0 Hz, 1H), 8.14-8.03 (m, 2H), 7.99 (dd, J=4.8, 1.6 Hz, 1H), 7.92 (d, J=8.4 Hz, 1H), 7.49-7.39 (m, 4H), 7.13 (dd, J=7.6, 1.2 Hz, 1H), 6.98 (br s, 2H), 6.68 (d, J=6.4 Hz, 1H), 6.37 (dd, J=7.6, 4.8 Hz, 1H), 3.95-3.71 (m, 1H), 3.58 (s, 2H), 2.86-2.80 (m, 2H), 2.20-2.12 (m, 2H), 1.97-1.84 (m, 2H), 1.57-1.45 (m, 2H).

### Example 595: 4-((1-(4-(2-(2-Aminopyridin-3-yl)-5-(4-fluoropyridin-3-yl)-3H-imidazo[4,5-b]pyridin-3-yl)benzyl)piperidin-4-yl)amino)pyrimidine-2-carbonitrile

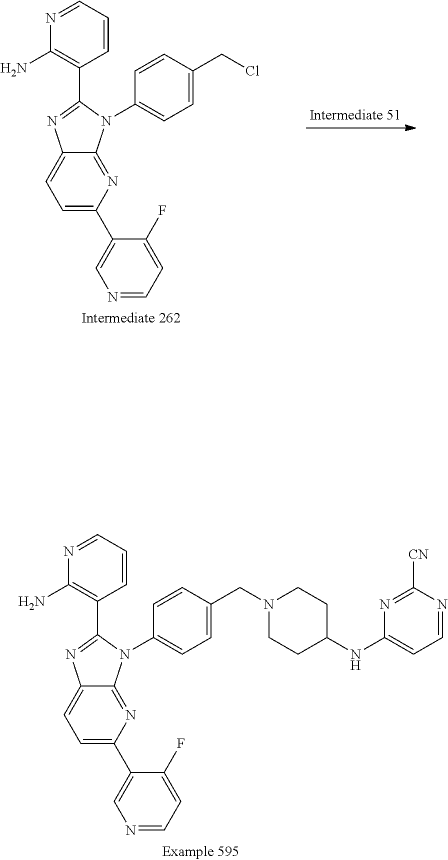 [SMILES: Nc1ncccc1-c1nc2ccc(-c3cnccc3F)nc2n1-c1ccc(CCl)cc1 ; N#Cc1nccc(NC2CCN(Cc3ccc(-n4c(-c5cccnc5N)nc5ccc(-c6cnccc6F)nc54)cc3)CC2)n1]

To a solution of Intermediate 262 (286 mg, 664 μmol) in DMF (4 mL) were added Intermediate 51 (149 mg, 731 μmol), NaI (19.9 mg, 133 μmol), and K₂CO₃ (276 mg, 2.0 mmol). The mixture was stirred at 25° C. for 12 hr under N₂. The mixture was diluted with H₂O (6 mL) and extracted with EtOAc (6 mL×3). The combined organic phases was washed with brine (6 mL), dried over anhydrous Na₂SO₄, filtered and concentrated. After purified by prep-HPLC (column: Welch Xtimate C18 150×25 mm×5 μm; mobile phase: [water (NH₄HCO₃)-ACN]; gradient: 42%-72% B over 7 min), 4-((1-(4-(2-(2-aminopyridin-3-yl)-5-(4-fluoropyridin-3-yl)-3H-imidazo[4,5-b]pyridin-3-yl)benzyl)piperidin-4-yl)amino)pyrimidine-2-carbonitrile (Example 595, 28.2 mg, yield: 6.8% for 3 steps) was obtained as a light yellow solid. MS: m/z=620.2 [M+Na]⁺. ¹H NMR (400 MHz, Dimethylsulfoxide-d₆) δ 8.98 (d, J=10.8 Hz, 1H), 8.61 (dd, J=7.2, 5.6 Hz, 1H), 8.34 (d, J=8.4 Hz, 1H), 8.12-7.97 (m, 3H), 7.83 (d, J=6.8 Hz, 1H), 7.50-7.42 (m, 5H), 7.18 (d, J=6.4 Hz, 1H), 7.01 (br s, 2H), 6.66 (d, J=6.0 Hz, 1H), 6.39 (dd, J=7.6, 4.8 Hz, 1H), 3.82-3.76 (m, 1H), 3.57 (s, 2H), 2.86-2.75 (m, 2H), 2.21-2.08 (m, 2H), 1.99-1.80 (m, 2H), 1.54-1.44 (m, 2H). ¹⁹F NMR (400 MHz, Dimethylsulfoxide-d₆) δ −108.465.

### Example 596: 4-((1-(4-(2-(2-Aminopyridin-3-yl)-5-(4-methylpyridin-2-yl)-3H-imidazo[4,5-b]pyridin-3-yl)benzyl)piperidin-4-yl)amino)pyrimidine-2-carbonitrile

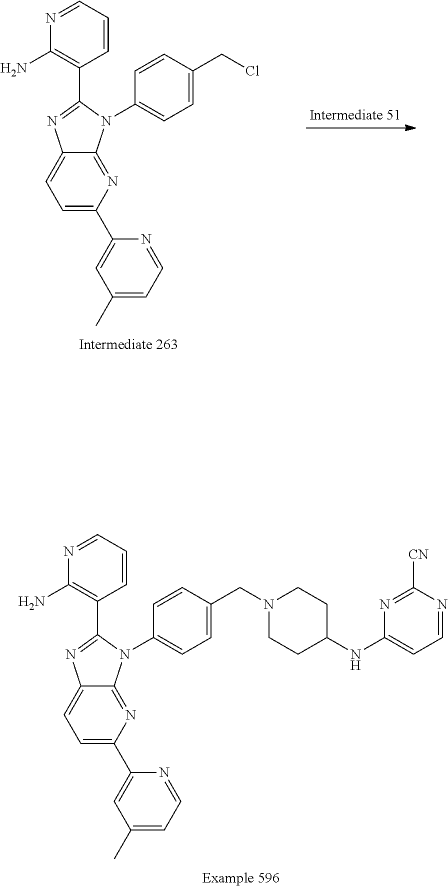 [SMILES: Cc1ccnc(-c2ccc3nc(-c4cccnc4N)n(-c4ccc(CCl)cc4)c3n2)c1 ; Cc1ccnc(-c2ccc3nc(-c4cccnc4N)n(-c4ccc(CN5CCC(Nc6ccnc(C#N)n6)CC5)cc4)c3n2)c1]

To a mixture of Intermediate 263 (261 mg, 611 μmol) and 4-(piperidin-4-ylamino)pyrimidine-2-carbonitrile (137 mg, 672 μmol) in DMF (3 mL) was added DIEA (395 mg, 3.06 mmol). The mixture was stirred at 20° C. for 1 hr. The mixture was diluted with H₂O (50 mL and extracted with EtOAc (50 mL×2). The combined organic layers were washed with brine (50 mL), dried over anhydrous Na₂SO₄, filtered and concentrated. After purified by prep-HPLC (column: C18 150×30 mm; mobile phase: [water (NH₃H₂O+NH₄HCO₃)-ACN]; gradient: 47%-77% B over 7 min), 4-((1-(4-(2-(2-aminopyridin-3-yl)-5-(4-methylpyridin-2-yl)-3H-imidazo[4,5-b]pyridin-3-yl)benzyl)piperidin-4-yl)amino)pyrimidine-2-carbonitrile (Example 596, 122.7 mg, yield: 33.8% for three steps) was obtained as an off white solid. MS: m/z=616.2 [M+Na]⁺. ¹H NMR (400 MHz, Dimethylsulfoxide-d₆) δ 8.53 (d, J=4.8 Hz, 1H), 8.46 (d, J=8.4 Hz, 1H), 8.30 (d, J=8.4 Hz, 1H), 8.07 (dd, J=14.0, 6.0 Hz, 2H), 8.02-7.97 (m, 2H), 7.53-7.45 (m, 4H), 7.23 (d, J=4.0 Hz, 1H), 7.17 (dd, J=7.6, 1.6 Hz, 1H), 7.03 (br s, 2H), 6.68 (d, J=6.0 Hz, 1H), 6.38 (dd, J=7.6, 4.8 Hz, 1H), 3.82 (s, 1H), 3.61 (s, 2H), 2.89-2.80 (m, 2H), 2.34 (s, 3H), 2.22-2.11 (m, 2H), 1.94-1.85 (m, 2H), 1.58-1.44 (m, 2H).

### Example 597: 4-((1-(4-(2-(2-aminopyridin-3-yl)-5-(6-(methoxy-d₃)pyridin-3-yl-2-d)-3H-imidazo[4,5-b]pyridin-3-yl)benzyl)piperidin-4-yl)amino)pyrimidine-2-carbonitrile

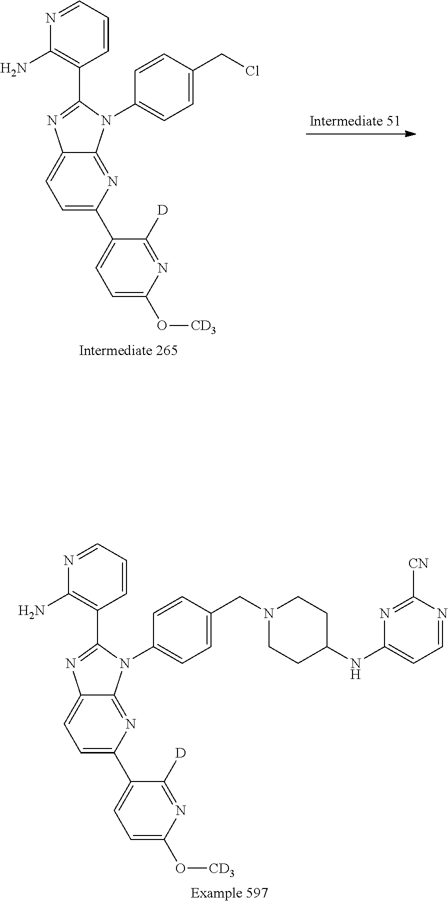 [SMILES: [2H]c1nc(OC([2H])([2H])[2H])ccc1-c1ccc2nc(-c3cccnc3N)n(-c3ccc(CCl)cc3)c2n1 ; [2H]c1nc(OC([2H])([2H])[2H])ccc1-c1ccc2nc(-c3cccnc3N)n(-c3ccc(CN4CCC(Nc5ccnc(C#N)n5)CC4)cc3)c2n1]

To a solution of Intermediate 265 (226 mg, 466 μmol, HCl salt) and Intermediate 51 (145 mg, 466 μmol, TFA) in DMF (2 mL) were added K₂CO₃ (322 mg, 2.33 mmol) and NaI (14 mg, 93.3 μmol). The mixture was stirred at 50° C. for 1 hr. The reaction mixture was quenched with H₂O (5 mL) at 25° C. and extracted with CH₂Cl₂ (15 mL×2). The combined organic layers were washed with brine (50 mL×3), dried over Na₂SO₄, filtered and concentrated under reduced pressure. After purified by prep-HPLC (column: Phenomenex C18 150×25 mm×10 μm; mobile phase: [water (NH₄HCO₃)-ACN]; gradient: 35%-65% B over 11 min), 4-((1-(4-(2-(2-aminopyridin-3-yl)-5-(6-(methoxy-d₃)pyridin-3-yl-2-d)-3H-imidazo[4,5-b]pyridin-3-yl)benzyl)piperidin-4-yl)amino)pyrimidine-2-carbonitrile (Example 597, 106 mg, yield: 37% for two steps) was obtained as a light yellow lyophilized powder. MS: m/z=613.3 [M+H]⁺. D %: 3D %=57.4%, 4D %=38.2%. ¹H NMR (400 MHz, Dimethylsulfoxide-d₆) δ 8.85 (s, 1H), 8.30 (dd, J=8.8, 2.8 Hz, 1H), 8.27 (d, J=8.4 Hz, 1H), 8.11-7.95 (m, 4H), 7.50-7.41 (m, 4H), 7.16 (dd, J=7.6, 1.6 Hz, 1H), 7.03 (br s, 2H), 6.96-6.87 (m, 1H), 6.67 (d, J=6.0 Hz, 1H), 6.38 (dd, J=7.6, 4.4 Hz, 1H), 3.90-3.75 (m, 1H), 3.59 (s, 2H), 2.89-2.79 (m, 2H), 2.21-2.10 (m, 2H), 1.95-1.83 (m, 2H), 1.57-1.44 (m, 2H).

### Example 598: 4-((1-(4-(2-(2-Aminopyridin-3-yl)-5-(6-(methoxy-d₃)pyridin-3-yl)-3H-imidazo[4,5-b]pyridin-3-yl)benzyl)piperidin-4-yl)amino)pyrimidine-2-carbonitrile

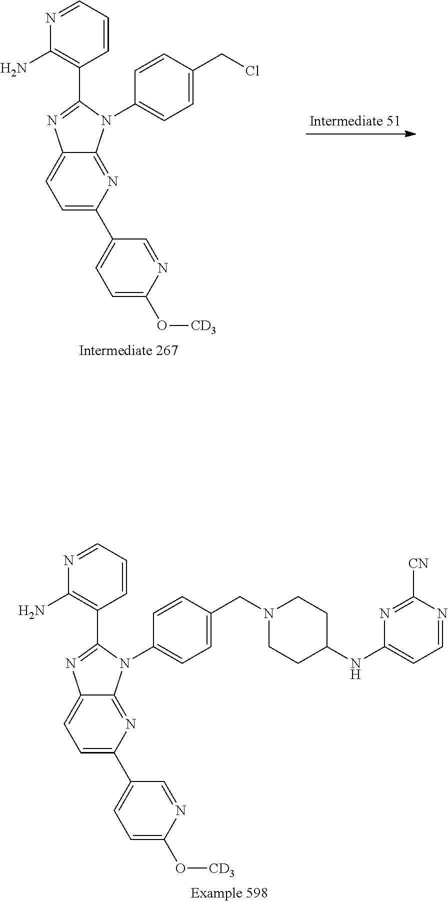 [SMILES: [2H]C([2H])([2H])Oc1ccc(-c2ccc3nc(-c4cccnc4N)n(-c4ccc(CCl)cc4)c3n2)cn1 ; [2H]C([2H])([2H])Oc1ccc(-c2ccc3nc(-c4cccnc4N)n(-c4ccc(CN5CCC(Nc6ccnc(C#N)n6)CC5)cc4)c3n2)cn1]

To a solution of Intermediate 267 (380 mg, 788 μmol, HCl salt) and Intermediate 51 (250 mg, 788 μmol, TFA) in DMF (5 mL) were added K₂CO₃ (544 mg, 3.94 mmol) and NaI (11.8 mg, 78.8 μmol). The mixture was stirred at 50° C. for 1 hr. The reaction mixture was quenched with H₂O (50 mL) at 25° C. and extracted with CH₂Cl₂ (50 mL×2). The combined organic layers were washed with brine (50 mL×3), dried over Na₂SO₄, filtered and concentrated under reduced pressure. After purified by silica gel flash chromatography (Eluent of 1%˜5% MeOH in CH₂Cl₂), 4-((1-(4-(2-(2-aminopyridin-3-yl)-5-(6-(methoxy-d₃)pyridin-3-yl)-3H-imidazo[4,5-b]pyridin-3-yl)benzyl)piperidin-4-yl)amino)pyrimidine-2-carbonitrile (Example 598, 104.7 mg, yield: 22% for two steps) was obtained as a brown solid. MS: m/z=613.3 [M+H]⁺. D %: 3D %=98.9%. ¹H NMR (400 MHz, Dimethylsulfoxide-d₆) δ 8.85 (d, J=2.0 Hz, 1H), 8.35-8.23 (m, 2H), 8.13-7.95 (m, 4H), 7.52-7.40 (m, 4H), 7.17-7.11 (m, 1H), 7.04 (s, 2H), 6.91 (d, J=8.4 Hz, 1H), 6.67 (d, J=6.0 Hz, 1H), 6.38 (dd, J=7.6, 4.8 Hz, 1H), 3.98-3.70 (m, 1H), 3.59 (s, 2H), 2.87-2.78 (m, 2H), 2.21-2.09 (m, 2H), 1.94-1.84 (m, 2H), 1.56-1.46 (m, 2H).

### Example 599: 2-(2-Aminopyridin-3-yl)-3-(4-((4-((2-cyanopyrimidin-4-yl)amino)piperidin-1-yl)methyl)phenyl)-5-hydroxy-3H-imidazo[4,5-h]pyridine-6-carbonitrile

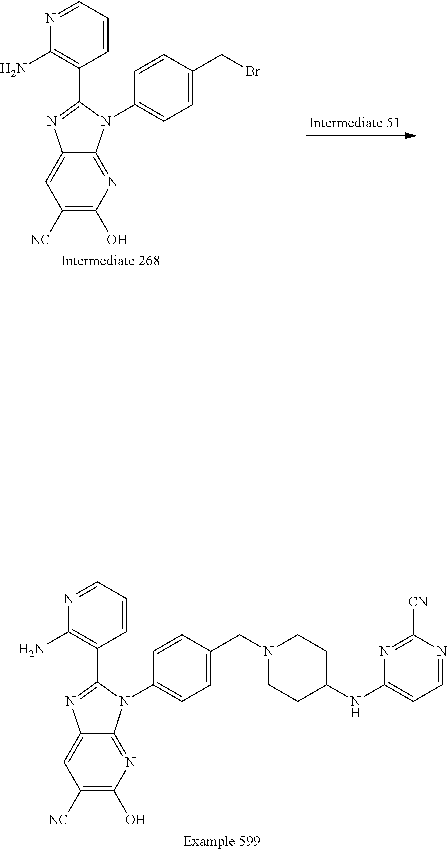 [SMILES: N#Cc1cc2nc(-c3cccnc3N)n(-c3ccc(CBr)cc3)c2nc1O ; N#Cc1nccc(NC2CCN(Cc3ccc(-n4c(-c5cccnc5N)nc5cc(C#N)c(O)nc54)cc3)CC2)n1]

To a solution of Intermediate 268 (350 mg, 830 μmol) and 4-(piperidin-4-ylamino)pyrimidine-2-carbonitrile (203 mg, 997 μmol) in DMF (3 mL) were added K₂CO₃ (459 mg, 3.3 mmol) and NaI (6.2 mg, 41 μmol). The mixture was stirred at 50° C. for 1 hr. The reaction mixture was quenched with H₂O (10 mL) at 25° C. and extracted with CH₂Cl₂ (15 mL×2). The combined organic layers were washed with brine (50 mL×3), dried over Na₂SO₄, filtered and concentrated under reduced pressure. After purified by silica gel flash chromatography (Eluent of 0˜10% MeOH in CH₂Cl₂), 2-(2-aminopyridin-3-yl)-3-(4-((4-((2-cyanopyrimidin-4-yl)amino)piperidin-1-yl)methyl)phenyl)-5-hydroxy-3H-imidazo[4,5-b]pyridine-6-carbonitrile (Example 599, 43.2 mg, yield: 9% for two steps) was obtained as a yellow solid. MS: m/z=544.2 [M+H]⁺. ¹H NMR (400 MHz, Dimethylsulfoxide-d₆) δ 8.57 (s, 1H), 8.18-8.04 (m, 2H), 7.98-7.92 (m, 1H), 7.52-7.33 (m, 5H), 7.09 (d, J=6.4 Hz, 1H), 6.91 (s, 2H), 6.69 (d, J=6.4 Hz, 1H), 6.34 (dd, J=7.6, 4.8 Hz, 1H), 3.88-3.78 (m, 1H), 3.65-3.59 (m, 2H), 2.86-2.80 (m, 2H), 2.27-2.15 (m, 2H), 1.94-1.85 (m, 2H), 1.56-1.47 (m, 2H).

### Example 600: 4-((1-(4-(2-(2-Amino-4-fluoropyridin-3-yl)-5-(5-fluoropyridin-2-yl)-3H-imidazo[4,5-b]pyridin-3-yl)benzyl)piperidin-4-yl)amino)pyrimidine-2-carbonitrile

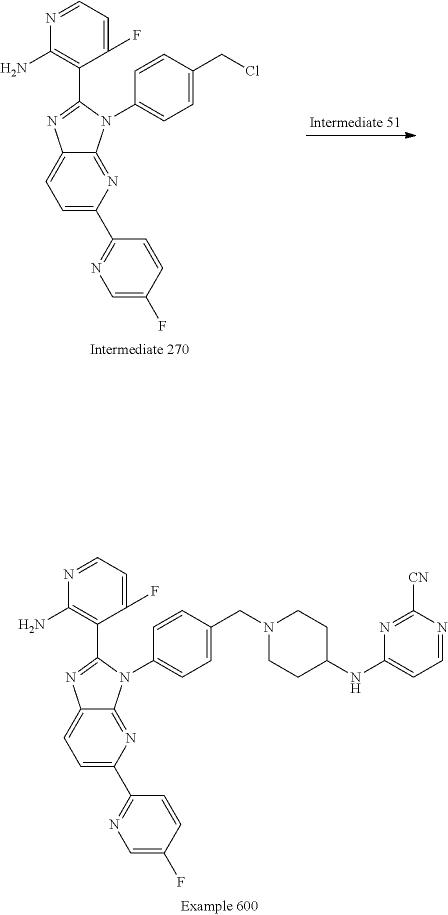 [SMILES: Nc1nccc(F)c1-c1nc2ccc(-c3ccc(F)cn3)nc2n1-c1ccc(CCl)cc1 ; N#Cc1nccc(NC2CCN(Cc3ccc(-n4c(-c5c(F)ccnc5N)nc5ccc(-c6ccc(F)cn6)nc54)cc3)CC2)n1]

To a solution of Intermediate 270 (20 mg, 41.2 μmol, HCl salt) and Intermediate 51 (13.1 mg, 41.2 μmol, TFA) in DMF (1 mL) were added K₂CO₃ (28.5 mg, 206 μmol) and NaI (618 μg, 4.12 μmol). The mixture was stirred at 50° C. for 1 hr. The reaction mixture was quenched with H₂O (5 mL) at 25° C. and extracted with CH₂Cl₂ (15 mL×2). The combined organic layers were washed with brine (50 mL×3), dried over Na₂SO₄, filtered and concentrated under reduced pressure. After purified by prep-TLC (SiO₂. CH₂Cl₂:MeOH=10:1), 4-((1-(4-(2-(2-amino-4-fluoropyridin-3-yl)-5-(5-fluoropyridin-2-yl)-3H-imidazo[4,5-b]pyridin-3-yl)benzyl)piperidin-4-yl)amino)pyrimidine-2-carbonitrile (Example 600, 7.9 mg, yield: 29% for three steps) was obtained as a yellow solid. MS: m/z=616.2 [M+H]⁺. ¹H NMR (400 MHz, Methanol-d₄) δ 8.56-8.48 (m, 2H), 8.37 (dd, J=8.8, 4.4 Hz, 1H), 8.28 (d, J=8.4 Hz, 1H), 8.08-7.98 (m, 1H), 7.66-7.61 (m, 1H), 7.55-7.47 (m, 4H), 7.35-7.30 (m, 1H), 6.61 (d, J=5.6 Hz, 1H), 6.38 (dd, J=8.8, 6.0 Hz, 1H), 4.05-3.93 (m, 1H), 3.73 (s, 2H), 3.06-2.98 (m, 2H), 2.43-2.31 (m, 2H), 2.10-2.03 (m, 2H), 1.68-1.60 (m, 2H). ¹⁹F NMR (400 MHz, Methanol-d₄) δ −102.89, −129.57.

### Example 601: 4-((1-(4-(5-(5-Fluoropyridin-2-yl)-2-(pyrimidin-5-yl)-3H-imidazo[4,5-b]pyridin-3-yl)benzyl)piperidin-4-yl)amino)pyrimidine-2-carbonitrile

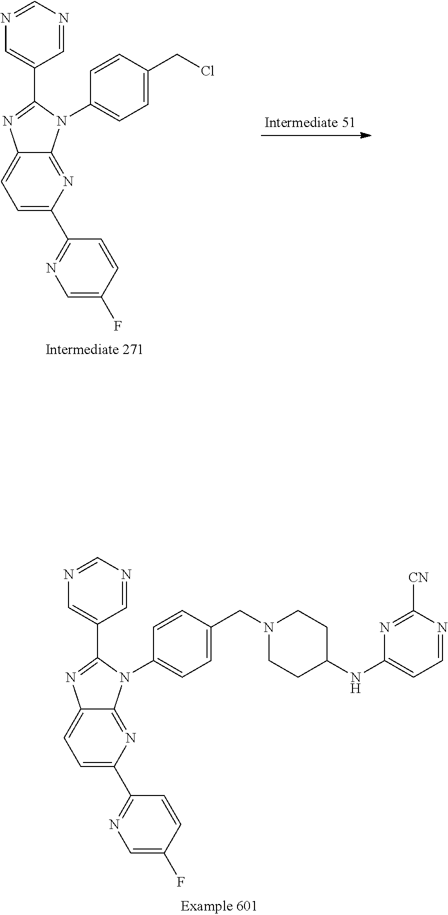 [SMILES: Fc1ccc(-c2ccc3nc(-c4cncnc4)n(-c4ccc(CCl)cc4)c3n2)nc1 ; N#Cc1nccc(NC2CCN(Cc3ccc(-n4c(-c5cncnc5)nc5ccc(-c6ccc(F)cn6)nc54)cc3)CC2)n1]

To a solution of Intermediate 271 (150 mg, 360 μmol, HCl) and Intermediate 51 (114 mg, 360 μmol, TFA) in DMF (8 mL) were added K₂CO₃ (149 mg, 1.08 mmol) and NaI (10.8 mg, 72.0 μmol). The mixture was stirred at 50° C. for 1 hr. The reaction mixture was quenched with H₂O (50 mL) at 25° C., diluted with CH₂Cl₂ (50 mL), and extracted with CH₂Cl₂ (50 mL×2). The combined organic layers were washed with brine (30 mL×3), dried over Na₂SO₄, filtered and concentrated under reduced pressure. After purified by prep-HPLC (column: Waters xbridge 150×25 mm×10 μm; mobile phase: [water (NH₄HCO₃)-ACN]; gradient: 35%-65% B over 10 min), 4-((1-(4-(5-(5-fluoropyridin-2-yl)-2-(pyrimidin-5-yl)-3H-imidazo[4,5-b]pyridin-3-yl)benzyl)piperidin-4-yl)amino)pyrimidine-2-carbonitrile (Example 601, 50.9 mg, yield: 24% for three steps) was obtained as a light yellow solid. MS: m/z=584.2 [M+H]⁺. ¹H NMR (400 MHz, Dimethylsulfoxide-d₆) δ 9.24 (s, 1H), 8.89 (s, 2H), 8.69 (d, J=2.8 Hz, 1H), 8.47-8.43 (m, 2H), 8.28-8.24 (m, 1H), 8.11-8.04 (m, 2H), 7.87-7.82 (m, 1H), 7.60-7.57 (m, 2H), 7.56-7.54 (m, 2H), 6.68 (d, J=5.2 Hz, 1H), 3.89-3.78 (m, 1H), 3.63 (s, 2H), 2.85 (d, J=11.2 Hz, 2H), 2.22-2.16 (m, 2H), 1.95-1.87 (m, 2H), 1.55-1.47 (m, 2H). ¹⁹F NMR (400 MHz, Dimethylsulfoxide-d₆) δ =−127.404.

### Example 602: 4-((1-(4-(2-(2-Aminopyridin-3-yl)-6-(5-fluoropyridin-2-yl)-1H-benzo[d]imidazol-1-yl)benzyl)piperidin-4-yl)amino)pyrimidine-2-carbonitrile

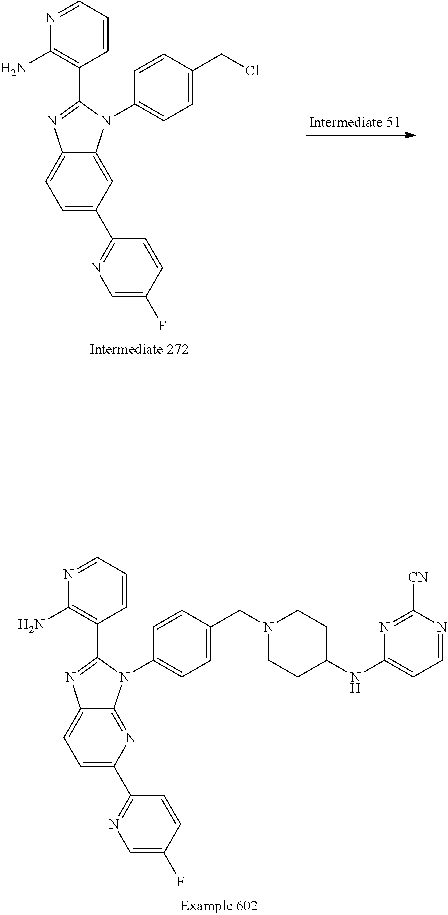 [SMILES: Nc1ncccc1-c1nc2ccc(-c3ccc(F)cn3)cc2n1-c1ccc(CCl)cc1 ; N#Cc1nccc(NC2CCN(Cc3ccc(-n4c(-c5cccnc5N)nc5ccc(-c6ccc(F)cn6)nc54)cc3)CC2)n1]

To a solution of Intermediate 272 (300 mg, 698 μmol, HCl salt) and Intermediate 51 (221 mg, 698 μmol, TFA) in DMF (5 mL) were added K₂CO₃ (289 mg, 2.09 mmol) and NaI (20.9 mg, 140 μmol). The mixture was stirred at 50° C. for 1 hr. The reaction mixture was quenched with H₂O (50 mL) at 25° C. and extracted with CH₂Cl₂ (50 mL×3). The combined organic layers were washed with brine (50 mL×3), dried over Na₂SO₄, filtered and concentrated under reduced pressure. After purified by prep-HPLC (column: Phenomenex C18 150×25 mm×10 μm; mobile phase: [water (NH₃H₂O)-ACN]; gradient: 38%-68%, over 11 min), 4-((1-(4-(2-(2-aminopyridin-3-yl)-6-(5-fluoropyridin-2-yl)-1H-benzo[d]imidazol-1-yl)benzyl)piperidin-4-yl)amino)pyrimidine-2-carbonitrile (Example 602, 55.0 mg, yield: 13% for two steps) was obtained as a light yellow lyophilized powder. MS: m/z=597.2 [M+H]⁺. ¹H NMR (400 MHz, Dimethylsulfoxide-d₆) δ 8.60 (d, J=2.8 Hz, 1H), 8.08 (dd, J=8.8, 4.4 Hz, 3H), 8.04-8.01 (m, 1H), 7.98 (dd, J=4.8, 1.8 Hz, 1H), 7.90-7.88 (m, 2H), 7.81-7.76 (m, 1H), 7.54-7.52 (m, 2H), 7.44 (d, J=8.4 Hz, 2H), 7.11-7.08 (m, 3H), 6.70-6.65 (m, 1H), 6.35 (dd, J=7.6, 4.8 Hz, 1H), 3.87-3.77 (m, 1H), 3.61 (s, 2H), 2.84-2.83 (m, 2H), 2.20-2.14 (m, 2H), 1.93-1.87 (m, 2H), 1.55-1.48 (m, 2H). ¹⁹F NMR (400 MHz, Dimethylsulfoxide-d₆) δ =−130.25.

### Example 603: 4-((1-(4-(2-(2-Aminopyridin-3-yl)-5-(5-fluoropyrimidin-2-yl)-3H-imidazo[4,5-b]pyridin-3-yl)benzyl)piperidin-4-yl)amino)pyrimidine-2-carbonitrile

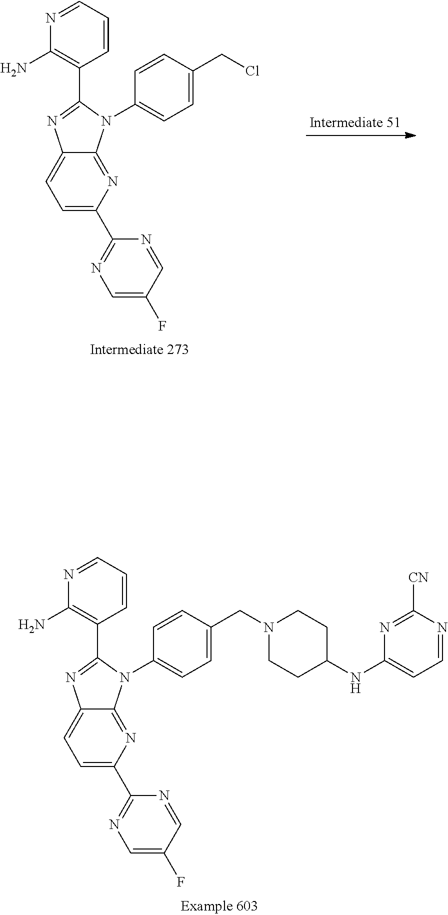 [SMILES: Nc1ncccc1-c1nc2ccc(-c3ncc(F)cn3)nc2n1-c1ccc(CCl)cc1 ; N#Cc1nccc(NC2CCN(Cc3ccc(-n4c(-c5cccnc5N)nc5ccc(-c6ncc(F)cn6)nc54)cc3)CC2)n1]

To a solution of Intermediate 273 (300 mg, 641 μmol, HCl) in DMF (5 mL) were added NaI (9.6 mg, 64 μmol) and K₂CO₃ (266 mg, 1.92 mmol). The mixture was stirred at 25° C. for 16 hr. The reaction mixture was diluted with H₂O (30 mL) and extracted with EtOAc (20 mL×3). The combined organic layers were washed with brine (30 mL), dried over Na₂SO₄, filtered and concentrated under reduced pressure. After purified by prep-HPLC (column: Phenomenex C18 150×25 mm×10 μm; mobile phase: [water (NH₃H₂O+NH₄HCO₃)-ACN]; gradient: 32%-52% B over 11 min), 4-((1-(4-(2-(2-aminopyridin-3-yl)-5-(5-fluoropyrimidin-2-yl)-3H-imidazo[4,5-b]pyridin-3-yl)benzyl)piperidin-4-yl)amino)pyrimidine-2-carbonitrile (Example 603, 47.1 mg, yield: 12% for three steps) was obtained as a yellow solid. MS: m/z=599.1 [M+H]⁺. ¹H NMR (400 MHz, Dimethysulfoxide-d₆) δ9.00 (s, 2H), 8.43 (d, J=8.4 Hz, 1H), 8.34 (d, J=8.4 Hz, 1H), 8.15-8.02 (m, 2H), 8.02-7.97 (m, 1H), 7.52-7.40 (m, 4H), 7.20-7.10 (m, 1H), 7.00 (br s, 2H), 6.67 (d, J=6.0 Hz, 1H), 6.37 (dd, J=7.6, 4.8 Hz, 1H), 3.89-3.74 (m, 1H), 3.59 (s, 2H), 2.92-2.77 (m, 2H), 2.23-2.10 (m, 2H), 1.95-1.80 (m, 2H), 1.61-1.42 (m, 2H). ¹⁹F NMR (400 MHz, Dimethysulfoxide-d₆) δ −139.07.

### Example 604: 4-((1-(4-(2-(2-Aminopyridin-3-yl)-5-(5-methylpyrimidin-2-yl)-3H-imidazo[4,5-b]pyridin-3-yl)benzyl)piperidin-4-yl)amino)pyrimidine-2-carbonitrile

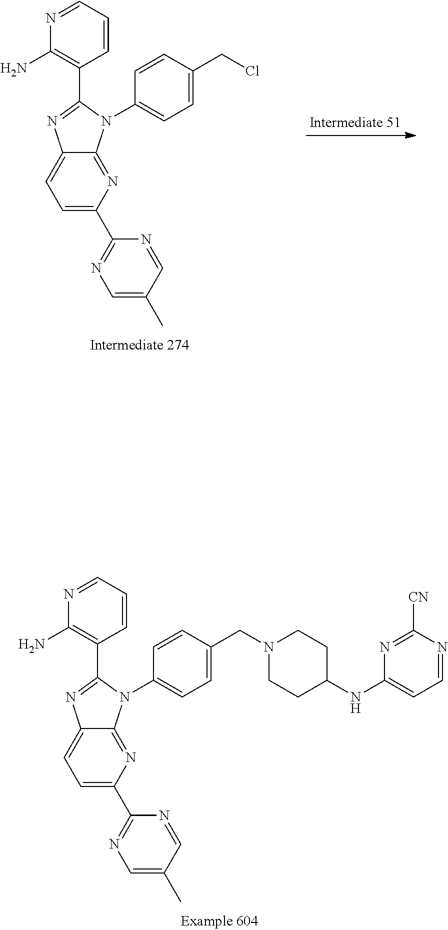 [SMILES: Cc1cnc(-c2ccc3nc(-c4cccnc4N)n(-c4ccc(CCl)cc4)c3n2)nc1 ; Cc1cnc(-c2ccc3nc(-c4cccnc4N)n(-c4ccc(CN5CCC(Nc6ccnc(C#N)n6)CC5)cc4)c3n2)nc1]

To a solution of Intermediate 274 (142 mg, 332 μmol) and Intermediate 51 (126 mg, 398 μmol, TFA) in DMF (2 mL) were added K₂CO₃ (229 mg, 1.66 mmol) and NaI (14.9 mg, 99.6 μmol). The mixture was stirred at 25° C. for 16 hr. The reaction mixture was poured into H₂O (15 mL) and extracted with EtOAc (15 mL×3). The combined organic layers were dried over anhydrous Na₂SO₄, filtered and concentrated under reduced pressure. After purified by silica gel flash chromatography (Eluent of 0%˜5% MeOH in CH₂Cl₂), 4-((1-(4-(2-(2-aminopyridin-3-yl)-5-(5-methylpyrimidin-2-yl)-3H-imidazo[4,5-b]pyridin-3-yl)benzyl)piperidin-4-yl)amino)pyrimidine-2-carbonitrile (Example 604, 34.9 mg, yield: 18% for three steps) was obtained as a yellow solid. MS: m/z=595.2 [M+H]⁺. ¹H NMR (400 MHz, Dimethysulfoxide-d₆) δ 8.84-8.66 (m, 2H), 8.46 (d, J=8.4 Hz, 1H), 8.32 (d, J=8.4 Hz, 1H), 8.19-8.02 (m, 2H), 8.01-7.96 (m, 1H), 7.61-7.28 (m, 4H), 7.22-7.08 (m, 1H), 7.00 (s, 2H), 6.77-6.57 (m, 1H), 6.44-6.30 (m, 1H), 3.89-3.70 (m, 1H), 3.60 (br s, 2H), 2.98-2.69 (m, 2H), 2.32 (s, 3H), 2.25-2.09 (m, 2H), 1.95-1.75 (m, 2H), 1.55-1.43 (m, 2H).

### Example 605: 4-((1-(4-(2-(2-Aminopyridin-3-yl)-5-(5-fluoropyrazin-2-yl)-3H-imidazo[4,5-b]pyridin-3-yl)benzyl)piperidin-4-yl)amino)pyrimidine-2-carbonitrile

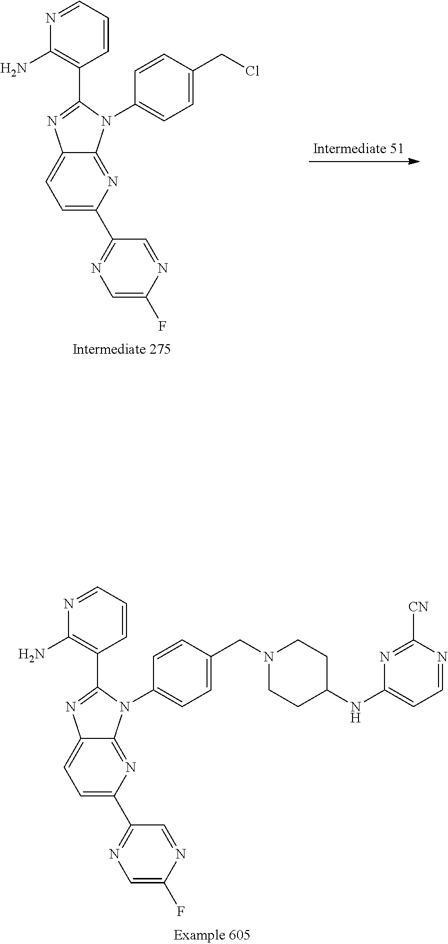 [SMILES: Nc1ncccc1-c1nc2ccc(-c3cnc(F)cn3)nc2n1-c1ccc(CCl)cc1 ; N#Cc1nccc(NC2CCN(Cc3ccc(-n4c(-c5cccnc5N)nc5ccc(-c6cnc(F)cn6)nc54)cc3)CC2)n1]

To a solution of Intermediate 275 (146 mg, 338 μmol, HCl salt) and 4-(piperidin-4-ylamino)pyrimidine-2-carbonitrile (118 mg, 372 μmol, TFA) in DMF (2 mL) were added K₂CO₃ (234 mg, 1.69 mmol) and NaI (15.2 mg, 101 μmol). The mixture was stirred at 25° C. for 1.5 hr. The reaction mixture was poured into H₂O (15 mL), extracted with EtOAc (15 mL×3). The combined organic layers were dried over anhydrous Na₂SO₄, filtered and concentrated under reduced pressure. After purified by prep-HPLC (column: Phenomenex C18 150×25 mm×10 μm; mobile phase: [water (NH₄HCO₃)-ACN]; gradient: 40%-60% B over 11 min), 4-((1-(4-(2-(2-aminopyridin-3-yl)-5-(5-fluoropyrazin-2-yl)-3H-imidazo[4,5-b]pyridin-3-yl)benzyl)piperidin-4-yl)amino)pyrimidine-2-carbonitrile (Example 605, 11 mg, yield: 5% for two steps) was obtained as a light yellow lyophilized powder. MS: m/z=599.3 [M+H]⁺. ¹H NMR (400 MHz, Dimethylsulfoxide-d₆) δ 9.04-8.91 (m, 1H), 8.85-8.73 (m, 1H), 8.39 (d, J=8.4 Hz, 1H), 8.32 (d, J=8.4 Hz, 1H), 8.15-7.97 (m, 3H), 7.53-7.47 (m, 4H), 7.16 (dd, J=7.6, 1.6 Hz, 1H), 7.02 (br s, 2H), 6.67 (d, J=6.0 Hz, 1H), 6.40 (dd, J=7.6, 4.8 Hz, 1H), 3.95-3.70 (m, 1H), 3.60 (s, 2H), 2.87-2.80 (m, 2H), 2.21-2.12 (m, 2H), 1.95-1.84 (m, 2H), 1.57-1.44 (m, 2H). ¹⁹FNMR (400 MHz, Dimethysulfoxide-dr) 6-82.705.

### Example 606: 4-((1-(4-(2-(2-Aminopyridin-3-yl)-5-(6-(fluoromethoxy)pyridin-3-yl)-3H-imidazo[4,5-b]pyridin-3-yl)benzyl)piperidin-4-yl)amino)pyrimidine-2-carbonitrile

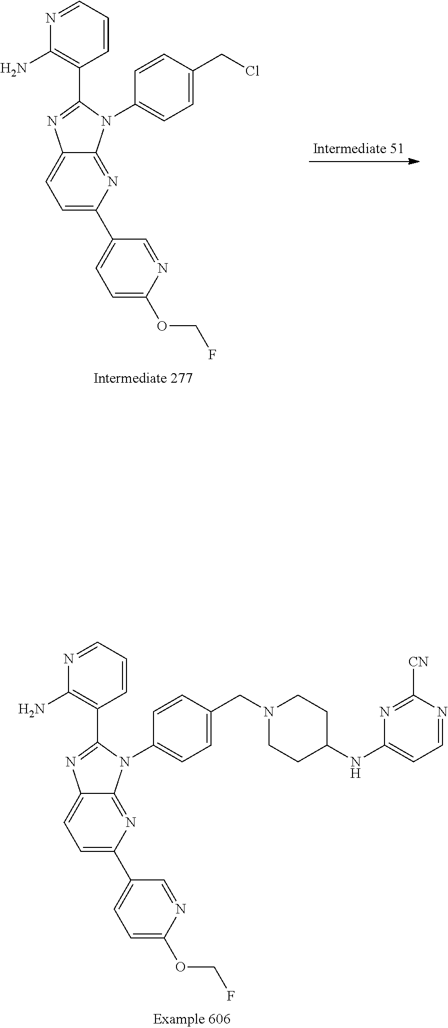 [SMILES: Nc1ncccc1-c1nc2ccc(-c3ccc(OCF)nc3)nc2n1-c1ccc(CCl)cc1 ; N#Cc1nccc(NC2CCN(Cc3ccc(-n4c(-c5cccnc5N)nc5ccc(-c6ccc(OCF)nc6)nc54)cc3)CC2)n1]

To a solution of Intermediate 277 (250 mg, 542 μmol, HCl salt) and Intermediate 51 (172 mg, 542 μmol, TFA) in DMF (3 mL) were added K₂CO₃ (300 mg, 1.17 mmol) and NaI (40.7 mg, 271 μmol). The mixture was stirred at 25° C. for 16 hr. The reaction mixture was quenched with H₂O (5 mL) at 25° C. and extracted with EtOAc (10 mL×3). The combined organic layers were washed with brine (50 mL×3), dried over Na₂SO₄, filtered and concentrated under reduced pressure. After purified by prep-HPLC (column: Phenomenex C18 150×25 mm×10 μm; mobile phase: [water (NH₄HCO₃)-ACN]; gradient: 35%-65% B over 11 min), 4-((1-(4-(2-(2-aminopyridin-3-yl)-5-(6-(fluoromethoxy)pyridin-3-yl)-3H-imidazo[4,5-b]pyridin-3-yl)benzyl)piperidin-4-yl)amino)pyrimidine-2-carbonitrile (Example 606, 145.3 mg, yield: 43% for three steps) was obtained as a yellow powder. MS: m/z=628.2 [M+H]⁺. ¹H NMR (400 MHz, Dimethylsulfoxide-d₆) δ 8.97-8.84 (m, 1H), 8.43 (d, J=8.8 Hz, 1H), 8.30 (d, J=8.4 Hz, 1H), 8.20-7.89 (m, 4H), 7.57-7.36 (m, 4H), 7.22-6.98 (m, 4H), 6.83-6.60 (m, 1H), 6.45-6.32 (m, 1H), 6.24-6.02 (m, 2H), 4.01-3.67 (m, 1H), 3.65-3.59 (s, 2H), 2.90-2.77 (m, 2H), 2.23-2.09 (m, 2H), 1.96-1.80 (m, 2H), 1.57-1.40 (m, 2H). ¹⁹F NMR (400 MHz, Dimethylsulfoxide-d₆) δ −154.35.

### Example 607: 4-((1-(4-(2-(2-Aminopyridin-3-yl)-5-(1-(fluoromethyl)-6-oxo-1,6-dihydropyridin-3-yl)-3H-imidazo[4,5-b]pyridin-3-yl)benzyl)piperidin-4-yl)amino)pyrimidine-2-carbonitrile

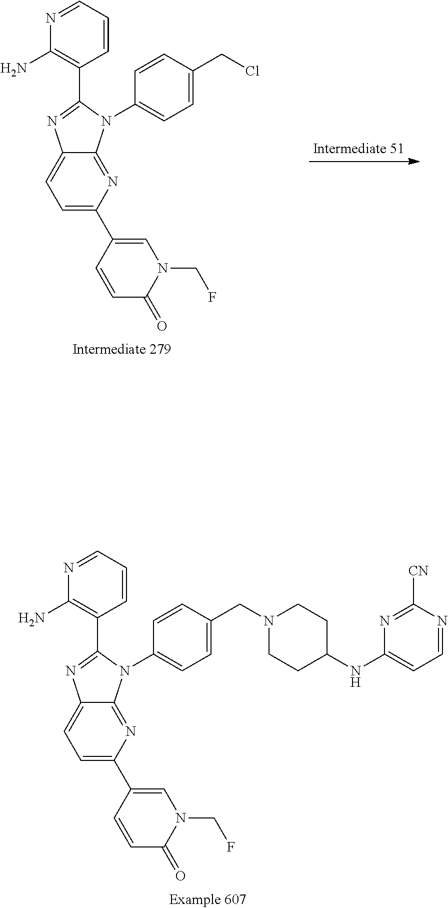 [SMILES: Nc1ncccc1-c1nc2ccc(-c3ccc(=O)n(CF)c3)nc2n1-c1ccc(CCl)cc1 ; N#Cc1nccc(NC2CCN(Cc3ccc(-n4c(-c5cccnc5N)nc5ccc(-c6ccc(=O)n(CF)c6)nc54)cc3)CC2)n1]

To a solution of Intermediate 279 (458 mg, 921 μmol, HCl salt) and Intermediate 51 (292 mg, 921 μmol) in DMF (3 mL) were added K₂CO₃ (509 mg, 3.68 mmol) and NaI (69 mg, 460 μmol). The mixture was stirred at 25° C. for 16 hr. The reaction mixture was quenched with H₂O (30 mL) at 25° C. and extracted with EtOAc (10 mL×3). The combined organic layers were washed with brine (20 mL×3), dried over Na₂SO₄, filtered and concentrated under reduced pressure. After purified by prep-HPLC (column: Daisogel SP ODS RPS 150×25 mm×5 μm; mobile phase: [water (NH₄HCO₃)-ACN]; gradient: 22%-52% B over 11 min), 4-((1-(4-(2-(2-aminopyridin-3-yl)-5-(1-(fluoromethyl)-6-oxo-1,6-dihydropyridin-3-yl)-3H-imidazo[4,5-b]pyridin-3-yl)benzyl)piperidin-4-yl)amino)pyrimidine-2-carbonitrile (Example 607, 291 mg, yield: 49% for three steps) was obtained as a yellow powder. MS: m/z=628.2 [M+H]⁺. ¹H NMR (400 MHz, Dimethylsulfoxide-d₆) δ 8.60 (d, J=2.4 Hz, 1H), 8.27 (d, J=8.4 Hz, 1H), 8.17 (dd, J=9.6, 2.4 Hz, 1H), 8.12-7.96 (m, 3H), 7.86 (d, J=8.4 Hz, 1H), 7.50-7.41 (m, 4H), 7.16-7.11 (m, 1H), 7.02 (s, 2H), 6.68 (d, J=6.4 Hz, 1H), 6.60 (d, J=9.6 Hz, 1H), 6.37 (dd, J=7.6, 4.8 Hz, 1H), 6.03 (d, J=50.8 Hz, 1H), 3.92-3.66 (m, 1H), 3.59 (s, 2H), 2.89-2.78 (m, 2H), 2.21-2.10 (m, 2H), 1.95-1.82 (m, 2H), 1.56-1.44 (m, 2H). ¹⁹F NMR (400 MHz, Dimethylsulfoxide-d₆) δ −172.673.

### Example 608: 4-((1-(4-(2-(2-Aminopyridin-3-yl)-5-(2-(methyl-d₃)-2H-1,2,3-triazol-4-yl)-3H-imidazo[4,5-b]pyridin-3-yl)benzyl)piperidin-4-yl)amino)pyrimidine-2-carbonitrile

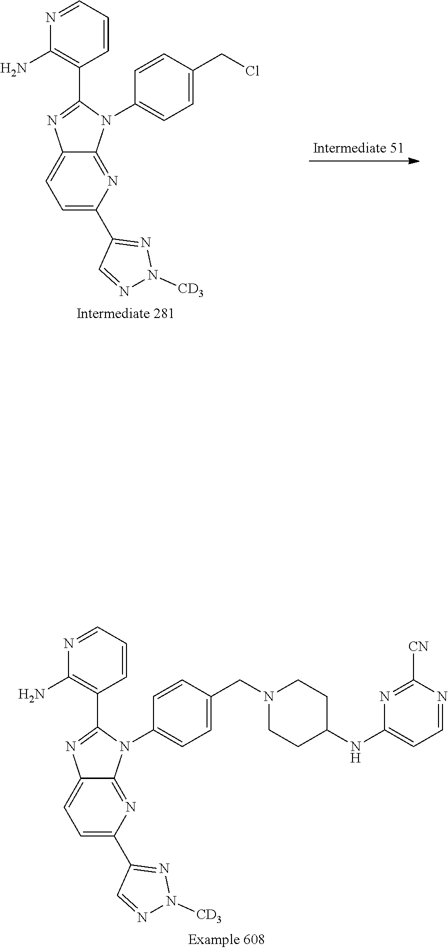 [SMILES: [2H]C([2H])([2H])n1ncc(-c2ccc3nc(-c4cccnc4N)n(-c4ccc(CCl)cc4)c3n2)n1 ; [2H]C([2H])([2H])n1ncc(-c2ccc3nc(-c4cccnc4N)n(-c4ccc(CN5CCC(Nc6ccnc(C#N)n6)CC5)cc4)c3n2)n1]

To a solution of Intermediate 281 (420 mg, 920 μmol, HCl salt) and Intermediate 51 (292 mg, 920 μmol, TFA) in DMF (5 mL) were added K₂CO₃ (636 mg, 4.6 mmol) and NaI (13.8 mg, 92 μmol). The mixture was stirred at 50° C. for 1 hr. The reaction mixture was quenched with H₂O (50 mL) at 25° C. and extracted with CH₂Cl₂ (50 mL×2). The combined organic layers were washed with brine (50 mL×3), dried over Na₂SO₄, filtered and concentrated under reduced pressure. After purified by silica gel flash chromatography (Eluent of 1%˜6% MeOH in CH₂Cl₂), 4-((1-(4-(2-(2-aminopyridin-3-yl)-5-(2-(methyl-d₃)-2H-1,2,3-triazol-4-yl)-3H-imidazo[4,5-b]pyridin-3-yl)benzyl)piperidin-4-yl)amino)pyrimidine-2-carbonitrile (Example 608, 301.2 mg, yield. 53% for two steps) was obtained as a green solid. MS: m/z=587.2 [M+H]⁺. ¹H NMR (400 MHz, Dimethylsulfoxide-d₆) δ 8.27 (d, J=8.4 Hz, 1H), 8.09-7.91 (m, 5H), 7.48-7.40 (m, 4H), 7.16-7.08 (m, 1H), 6.93 (s, 2H), 6.67 (d, J=6.0 Hz, 1H), 6.37 (dd, J=7.6, 4.8 Hz, 1H), 3.85-3.77 (m, 1H), 3.58 (s, 2H), 2.86-2.80 (m, 2H), 2.19-2.12 (m, 2H), 1.93-1.84 (m, 2H), 1.53-1.46 (m, 2H).

### Example 609: 4-((1-(4-(2-(2-Aminopyridin-3-yl)-5-(2-ethyl-2H-1,2,3-triazol-4-yl)-3H-imidazo[4,5-b]pyridin-3-yl)benzyl)piperidin-4-yl)amino)pyrimidine-2-carbonitrile

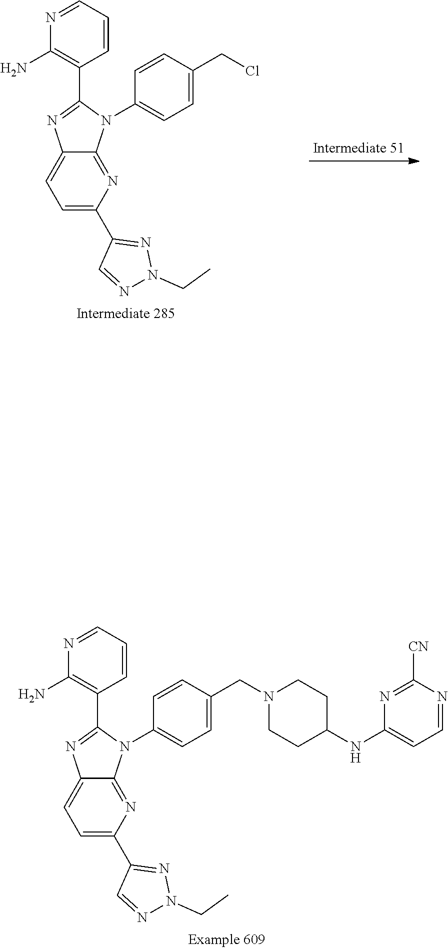 [SMILES: CCn1ncc(-c2ccc3nc(-c4cccnc4N)n(-c4ccc(CCl)cc4)c3n2)n1 ; CCn1ncc(-c2ccc3nc(-c4cccnc4N)n(-c4ccc(CN5CCC(Nc6ccnc(C#N)n6)CC5)cc4)c3n2)n1]

To a mixture of Intermediate 285 (171 mg, 397 μmol) and Intermediate 51 (88.7 mg, 437 μmol) in DMF (4 mL) were added NaI (11.9 mg, 79.4 μmol) and K₂CO₃ (165 mg, 1.19 mmol). The reaction mixture was stirred at 20° C. for 12 hr. The mixture was diluted with H₂O (20 mL) and extracted with EtOAc (20 mL×2). The combined organic layers were washed with brine (10 mL), dried over anhydrous Na₂SO₄, filtered and concentrated. After purified by prep-HPLC (column: C18 150×30 mm; mobile phase: [water(NH₃H₂O+NH₄HCO₃)-ACN]; gradient: 45%-75% B over 7 min), 4-((1-(4-(2-(2-aminopyridin-3-yl)-5-(2-ethyl-2H-1,2,3-triazol-4-yl)-3H-imidazo[4,5-b]pyridin-3-yl)benzyl)piperidin-4-yl)amino)pyrimidine-2-carbonitrile (Example 609, 66 mg, 110 μmol, yield: 28% for three steps) was obtained as a yellow solid. MS: m/z=620.2 [M+Na]˜. ¹H NMR (400 MHz, Dimethylsulfoxide-d₆) δ 8.28 (d, J=8.0 Hz, 1H), 8.13-8.04 (m, 3H), 8.02-7.92 (m, 2H), 7.51-7.40 (m, 4H), 7.14 (d, J=6.4 Hz, 1H), 6.98 (s, 2H), 6.68 (d, J=5.6 Hz, 1H), 6.37 (dd, J=7.6, 4.8 Hz, 1H), 4.57-4.46 (m, 2H), 3.89-3.77 (m, 1H), 3.64-3.55 (m, 2H), 2.89-2.77 (m, 2H), 2.22-2.09 (m, 2H), 1.95-1.84 (m, 2H), 1.55-1.44 (m, 5H).

### Example 610: 4-((1-(4-(2-(2-Aminopyridin-3-yl)-5-(2-isopropyl-2H-1,2,3-triazol-4-yl)-3H-imidazo[4,5-b]pyridin-3-yl)benzyl)piperidin-4-yl)amino)pyrimidine-2-carbonitrile

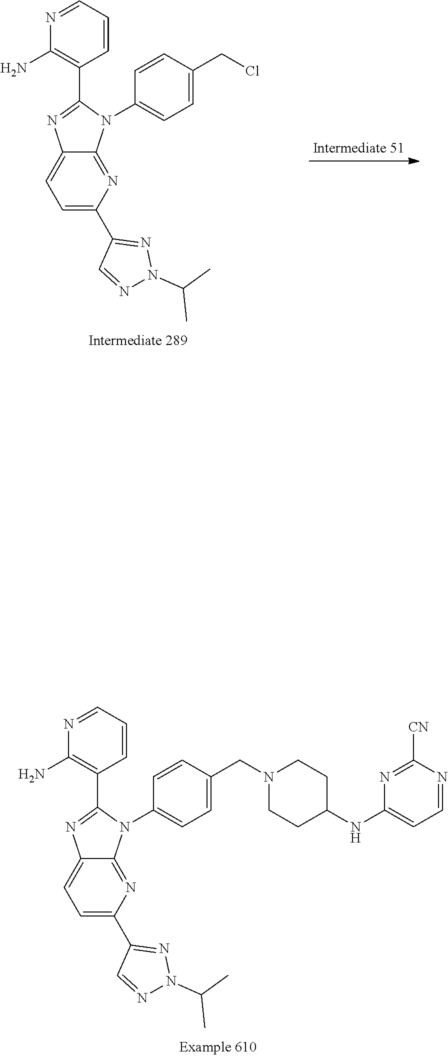 [SMILES: CC(C)n1ncc(-c2ccc3nc(-c4cccnc4N)n(-c4ccc(CCl)cc4)c3n2)n1 ; CC(C)n1ncc(-c2ccc3nc(-c4cccnc4N)n(-c4ccc(CN5CCC(Nc6ccnc(C#N)n6)CC5)cc4)c3n2)n1]

To a solution of Intermediate 289 (146 mg, 328 μmol) and 4-(piperidin-4-ylamino)pyrimidine-2-carbonitrile (73.4 mg, 361 μmol) in DMF (5 mL) were added K₂CO₃ (136 mg, 984 μmol) and NaI (9.84 mg, 65.6 μmol) at 20° C. The reaction mixture was stirred at 20° C. for 12 hr. The mixture was diluted with H₂O (10 mL) and extracted with EtOAc (10 mL×2). The combined organic layers were washed with brine (10 mL), dried over anhydrous Na₂SO₄, filtered and concentrated. After purified by prep-HPLC (column: C18 150×30 mm; mobile phase: [water(NH₃H₂O+NH₄HCO₃)-ACN]; gradient: 51%-81% B over 7 min), 4-((1-(4-(2-(2-aminopyridin-3-yl)-5-(2-isopropyl-2H-1,2,3-triazol-4-yl)-3H-imidazo[4,5-b]pyridin-3-yl)benzyl)piperidin-4-yl)amino)pyrimidine-2-carbonitrile (Example 610, 53.3 mg, yield: 26.6% for three steps) was obtained as an off-white solid. MS: m/z=612.3 [M+H]⁺. ¹H NMR (400 MHz, Dimethylsulfoxide-d₆) δ 8.27 (d, J=8.4 Hz, 1H), 8.15-8.03 (m, 3H), 7.99 (dd, J=4.8, 1.6 Hz, 1H), 7.95 (d, J=8.4 Hz, 1H), 7.50-7.41 (m, 4H), 7.14 (dd, J=7.6, 1.6, Hz, 1H), 7.00 (s, 2H), 6.67 (d, J=6.0 Hz, 1H), 6.37 (dd, J=8.0, 4.8 Hz, 1H), 4.93-4.82 (m, 1H), 3.82-3.81 (m, 1H), 3.59 (s, 2H), 2.87-2.78 (m, 2H), 2.23-2.09 (m, 2H), 1.96-1.78 (m, 2H), 1.58-1.43 (m, 8H).

### Example 611: 4-((1-(4-(2-(2-Aminopyridin-3-yl)-5-(1-isopropyl-11H-1,2,3-triazol-4-yl)-3H-imidazo[4,5-b]pyridin-3-yl)benzyl)piperidin-4-yl)amino)pyrimidine-2-carbonitrile

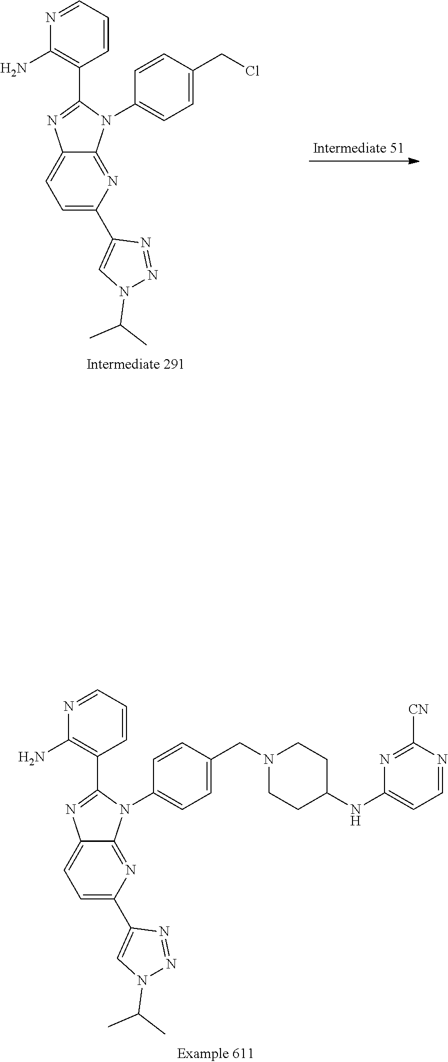 [SMILES: CC(C)n1cc(-c2ccc3nc(-c4cccnc4N)n(-c4ccc(CCl)cc4)c3n2)nn1 ; CC(C)n1cc(-c2ccc3nc(-c4cccnc4N)n(-c4ccc(CN5CCC(Nc6ccnc(C#N)n6)CC5)cc4)c3n2)nn1]

To a solution of Intermediate 291 (220 mg, 494 μmol, HCl salt) and Intermediate 51 (111 mg, 544 μmol) in DMF (8 mL) were added NaI (14.8 mg, 98.9 μmol) and K₂CO₃ (205 mg, 1.48 mmol) at 20° C. The reaction mixture was stirred at 20° C. for 12 hr. The mixture was diluted with H₂O (20 mL) and extracted with EtOAc (20 mL×2). The combined organic layers were washed with brine (20 mL), dried over anhydrous Na₂SO₄, filtered and concentrated. After purified by prep-HPLC (column: C18 150×30 mm; mobile phase: [water (NH₃H₂O+NH₄HCO₃)-ACN]; gradient: 45%-75% B over 7 min), 4-((1-(4-(2-(2-aminopyridin-3-yl)-5-(1-isopropyl-1H-1,2,3-triazol-4-yl)-3H-imidazo[4,5-b]pyridin-3-yl)benzyl)piperidin-4-yl)amino)pyrimidine-2-carbonitrile (Intermediate 611, 119 mg, yield: 39% for three steps) was obtained as an off-white solid. MS: m/z=612.3 [M+H]⁺. ¹H NMR (400 MHz, Dimethylsulfoxide-d₆) δ 8.42 (s, 1H), 8.28 (d, J=8.4 Hz, 1H), 8.12-8.04 (m, 3H), 7.98 (dd, J=4.8, 1.6 Hz, 1H), 7.49-7.41 (m, 4H), 7.11 (dd, J=7.6, 1.6 Hz, 1H), 6.97 (br s, 2H), 6.68 (d, J=6.0 Hz, 1H), 6.36 (dd, J=7.6, 4.8 Hz, 1H), 4.93-4.82 (m, 1H), 3.87-3.76 (m, 1H), 3.59 (s, 2H), 2.89-2.79 (m, 2H), 2.22-2.11 (m, 2H), 1.94-1.79 (m, 2H), 1.54-1.47 (m, 8H).

### Example 612: 4-((1-(4-(2-(2-Aminopyridin-3-yl)-5-(2-methyl-5-(methyl-d₃)-2H-1,2,3-triazol-4-yl)-3H-imidazo[4,5-b]pyridin-3- yl)benzyl)piperidin-4-yl)amino)pyrimidine-2-carbonitrile

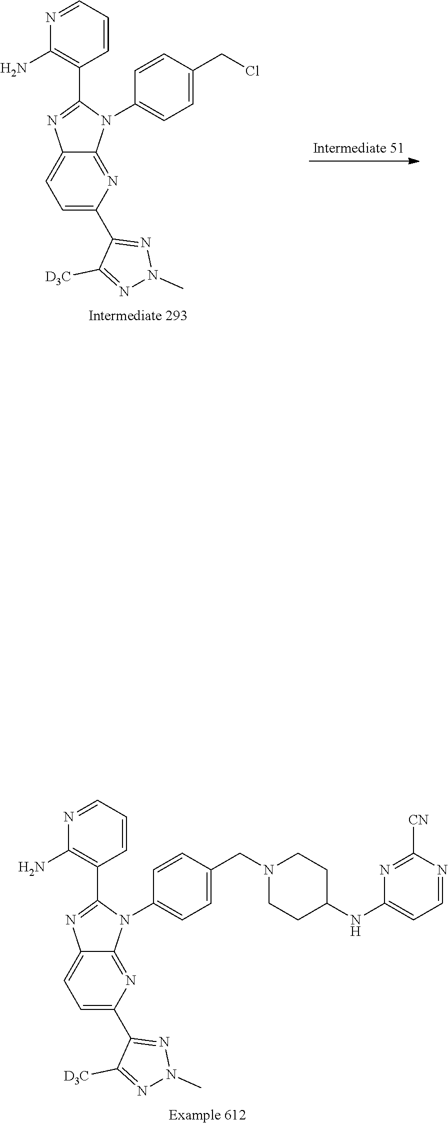 [SMILES: [2H]C([2H])([2H])c1nn(C)nc1-c1ccc2nc(-c3cccnc3N)n(-c3ccc(CCl)cc3)c2n1 ; [2H]C([2H])([2H])c1nn(C)nc1-c1ccc2nc(-c3cccnc3N)n(-c3ccc(CN4CCC(Nc5ccnc(C#N)n5)CC4)cc3)c2n1]

To a solution of Intermediate 293 (170 mg, 359 μmol, HCl salt) and Intermediate 51 (114 mg, 359 μmol, TFA salt) in DMF (2 mL) were added K₂CO₃ (199 mg, 1.44 mmol) and NaI (54 mg, 359 μmol). The mixture was degassed and purged with N₂ three times and stirred at 25° C. for 1 hr under N₂. The reaction mixture was filtered and concentrated under reduced pressure. After purified by prep-HPLC (column: Waters xbridge 150×25 mm 10 μm; mobile phase: [water (NH₄HCO₃)-ACN]; gradient: 34%-64% B over 11 min), 4-((1-(4-(2-(2-aminopyridin-3-yl)-5-(2-methyl-5-(methyl-d₃)-2H-1,2,3-triazol-4-yl)-3H-imidazo[4,5-b]pyridin-3-yl)benzyl)piperidin-4-yl)amino)pyrimidine-2-carbonitrile (Example 612, 19.7 mg, yield: 8.9% for three steps) was obtained as a yellow solid. MS: m/z=601.3 [M+H]⁺. D %: 3D %=100%, ¹H NMR (400 MHz, Dimethylsulfoxide-d₆) δ 8.25 (d, J=8.0 Hz, 1H), 8.12-8.03 (m, 2H), 8.02-8.00 (m, 1H), 7.91 (d, J=8.4 Hz, 1H), 7.51-7.40 (m, 4H), 7.25 (dd, J=8.0, 1.2 Hz, 1H), 7.09 (br s, 2H), 6.67 (d, J=6.4 Hz, 1H), 6.41 (dd, J=7.6, 4.8, Hz, 1H), 4.12 (s, 3H), 3.85-3.75 (m, 1H), 3.58 (s, 2H), 3.21-3.09 (m, 2H), 2.83-2.76 (m, 2H), 2.17-2.08 (m, 2H), 1.93-1.83 (m, 2H).

### Example 613: 4-((1-(4-(2-(2-Aminopyridin-3-yl)-5-(2H-1,2,3-triazol-4-yl)-3H-imidazo[4,5-b]pyridin-3-yl)benzyl)piperidin-4-yl)amino)pyrimidine-2-carbonitrile

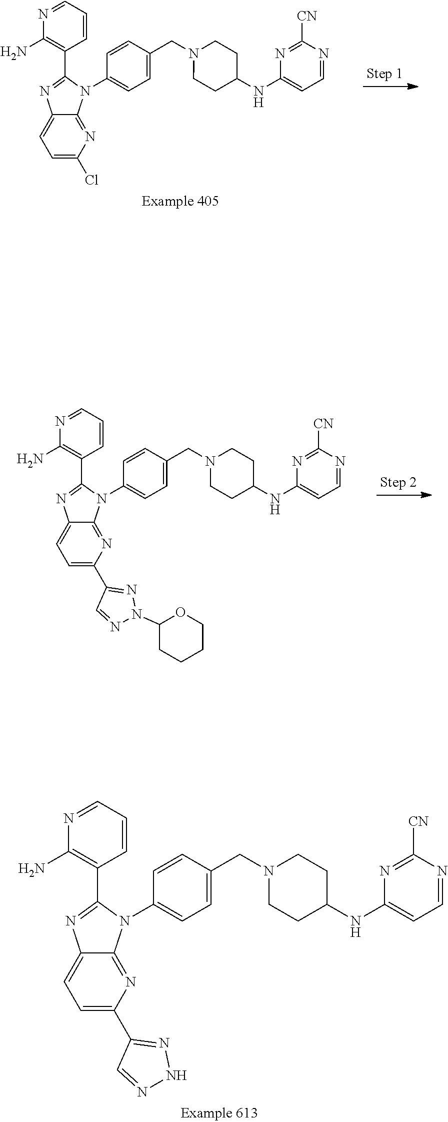 [SMILES: N#Cc1nccc(NC2CCN(Cc3ccc(-n4c(-c5cccnc5N)nc5ccc(Cl)nc54)cc3)CC2)n1 ; N#Cc1nccc(NC2CCN(Cc3ccc(-n4c(-c5cccnc5N)nc5ccc(-c6cnn(C7CCCCO7)n6)nc54)cc3)CC2)n1 ; N#Cc1nccc(NC2CCN(Cc3ccc(-n4c(-c5cccnc5N)nc5ccc(-c6cn[nH]n6)nc54)cc3)CC2)n1]

### Step 1: 4-((1-(4-(2-(2-Aminopyridin-3-yl)-5-(2-(tetrahydro-2H-pyran-2-yl)-2H-1,2,3-triazol-4-yl)-3H-imidazo[4,5-b]pyridin-3-yl)benzyl)piperidin-4-yl)amino)pyrimidine-2-carbonitrile

A mixture of Example 405 (250 mg, 466 μmol), 2-(tetrahydro-2H-pyran-2-yl)-4-(4,4,5,5-tetramethyl-1,3,2-dioxaborolan-2-yl)-2H-1,2,3-triazole (143 mg, 512 μmol), Pd(dppf)Cl₂ (34.1 mg, 46.5 μmol), and Cs₂CO₃ (455 mg, 1.40 mmol) in H₂O (0.5 mL) and 1,4-dioxane (2 mL) was degassed and purged with N₂ three times. The mixture was stirred at 100° C. for 16 hr under N₂ atmosphere. The reaction mixture was poured into H₂O (15 mL) and extracted with EtOAc (15 mL×3). The combined organic layers were dried over anhydrous Na₂SO₄, filtered and concentrated under reduced pressure. 4-((1-(4-(2-(2-Aminopyridin-3-yl)-5-(2-(tetrahydro-2H-pyran-2-yl)-2H-1,2,3-triazol-4-yl)-3H-imidazo[4,5-b]pyridin-3-yl)benzyl)piperidin-4-yl)amino)pyrimidine-2-carbonitrile (285 mg) was obtained as a brown solid. MS: m/z=654.4 [M+H]⁺

### Step 2: 4-((1-(4-(2-(2-Aminopyridin-3-yl)-5-(2H-1,2,3-triazol-4-yl)-3H-imidazo[4,5-b]pyridin-3-yl)benzyl)piperidin-4-yl)amino)pyrimidine-2-carbonitrile

To a solution of 4-((1-(4-(2-(2-aminopyridin-3-yl)-5-(2-(tetrahydro-2H-pyran-2-yl)-2H-1,2,3-triazol-4-yl)-3H-imidazo[4,5-b]pyridin-3-yl)benzyl)piperidin-4-yl)amino)pyrimidine-2-carbonitrile (285 mg, 436 μmol) in CH₂Cl₂ (4 mL) was added TFA (2 mL). The mixture was stirred at 25° C. for 2 hr. The pH of reaction mixture was adjusted to 8 with aq. Sat. NaHCO₃. The mixture was extracted with CH₂Cl₂ (20 mL×3). The combined organic layers were dried over Na₂SO₄, filtered and concentrated under reduced pressure. After purified by prep-HPLC(column: Phenomenex C18 150×25 mm×10 μm; mobile phase: [water (NH₄HCO₃)-ACN]; gradient: 18%-48% B over 11 min), 4-((1-(4-(2-(2-aminopyridin-3-yl)-5-(2H-1,2,3-triazol-4-yl)-3H-imidazo[4,5-b]pyridin-3-yl)benzyl)piperidin-4-yl)amino)pyrimidine-2-carbonitrile (Example 613, 12.2 mg, yield: 5% for two steps) was obtained as an off white solid. MS: m/z=570.3 [M+H]⁺. 1H NMR (400 MHz, Methanol-d₄) δ 8.30-8.13 (m, 2H), 8.11-8.05 (m, 1H), 8.04-7.95 (m, 2H), 7.54 (d, J=8.4 Hz, 2H), 7.44 (d, J=8.4 Hz, 2H), 7.30 (dd, J=7.6, 1.6 Hz, 1H), 6.66-6.53 (m, 1H), 6.46 (dd, J=8.0, 5.2 Hz, 1H), 4.07-3.84 (m, 1H), 3.67 (br s, 2H), 3.07-2.88 (m, 2H), 2.38-2.17 (m, 2H), 2.10-1.90 (m, 2H), 1.69-1.57 (m, 2H).

### Example 614: 4-((1-(4-(2-(2-Aminopyridin-3-yl)-5-(2,5-dimethyl-2H-1,2,3-triazol-4-yl)-3H-imidazo[4,5-b]pyridin-3-yl)benzyl)piperidin-4-yl)amino)pyrimidine-2-carbonitrile

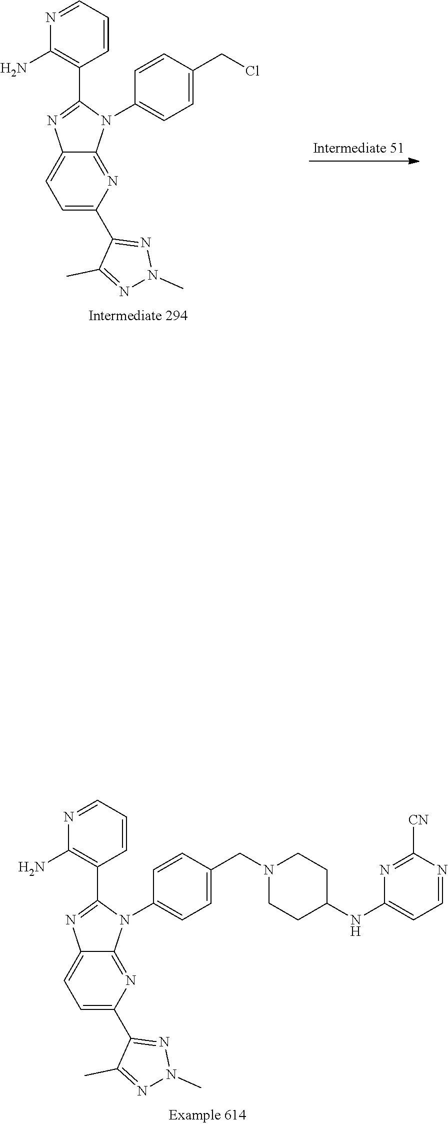 [SMILES: Cc1nn(C)nc1-c1ccc2nc(-c3cccnc3N)n(-c3ccc(CCl)cc3)c2n1 ; Cc1nn(C)nc1-c1ccc2nc(-c3cccnc3N)n(-c3ccc(CN4CCC(Nc5ccnc(C#N)n5)CC4)cc3)c2n1]

To a mixture of Intermediate 294 (230 mg, 534 μmol) and Intermediate 51 (119 mg, 587 μmol) in DMF (2 mL) were added NaI (16.0 mg, 106 μmol) and K₂CO₃ (221 mg, 1.60 mmol), the mixture was stirred at 20° C. for 12 hr. The mixture was diluted with H₂O (50 mL) and extracted with EtOAc (30 mL×2). The combined organic layers were washed with brine (30 mL), dried over anhydrous Na₂SO₄, filtered and concentrated. After purified by prep-HPLC (column: C18 150×30 mm; mobile phase: [water(NH₃H₂O+NH₄HCO₃)-ACN]; gradient: 48%-78% B over 7 min), 4-((1-(4-(2-(2-aminopyridin-3-yl)-5-(2,5-dimethyl-2H-1,2,3-triazol-4-yl)-3H-imidazo[4,5-b]pyridin-3-yl)benzyl)piperidin-4-yl)amino)pyrimidine-2-carbonitrile (Example 614, 78.2 mg, yield: 24% for two steps) was obtained as a white solid. MS: m/z=598.3 [M+H]⁺. ¹H NMR (400 MHz, Dimethylsulfoxide-d₆) δ 8.25 (d, J=8.4 Hz, 1H), 8.10-8.00 (m, 3H), 7.91 (d, J=8.4 Hz, 1H), 7.49-7.43 (m, 4H), 7.25 (dd, J=8.0, 2.0 Hz, 1H), 7.09 (s, 2H), 6.67 (d, J=5.6 Hz, 1H), 6.41 (dd, J=7.6, 4.8 Hz, 1H), 4.12 (s, 3H), 3.87-3.73 (m, 1H), 3.58 (s, 2H), 2.84-2.75 (m, 2H), 2.34 (s, 3H), 2.18-2.06 (m, 2H), 1.93-1.82 (m, 2H), 1.56-1.42 (m, 2H).

### Example 615: 4-((1-(4-(2-(2-Aminopyridin-3-yl)-5-(2-cyclopropyl-2H-1,2,3-triazol-4-yl)-3H-imidazo[4,5-b]pyridin-3-yl)benzyl)piperidin-4-yl)amino)pyrimidine-2-carbonitrile

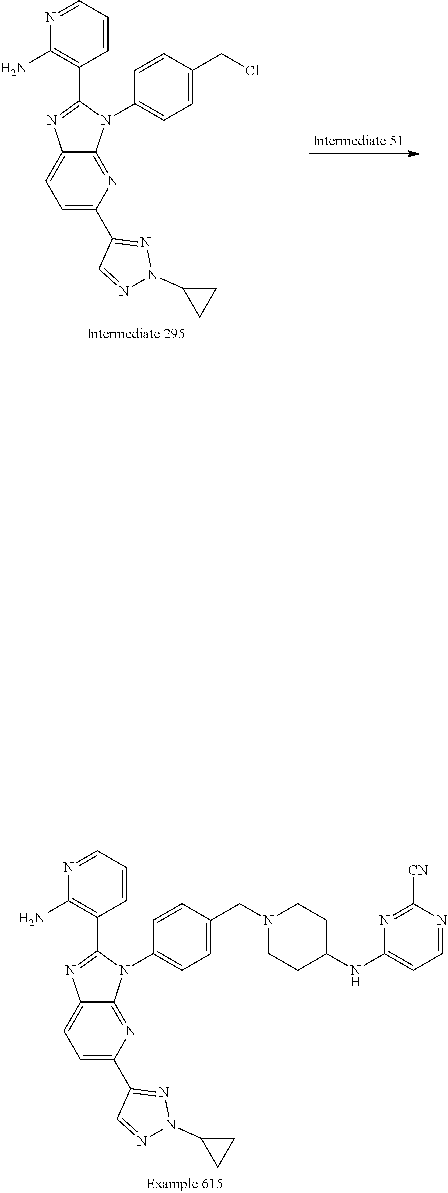 [SMILES: Nc1ncccc1-c1nc2ccc(-c3cnn(C4CC4)n3)nc2n1-c1ccc(CCl)cc1 ; N#Cc1nccc(NC2CCN(Cc3ccc(-n4c(-c5cccnc5N)nc5ccc(-c6cnn(C7CC7)n6)nc54)cc3)CC2)n1]

To a solution of Intermediate 295 (170 mg, 384 μmol) and Intermediate 51 (86 mg, 422 μmol) in DMF (3 mL) were added K₂CO₃ (159 mg, 1.15 mmol) and NaI (11.5 mg, 76.8 μmol) at 20° C. The reaction mixture was stirred at 20° C. for 12 hr. The reaction mixture was diluted with H₂O (20 ml) and extracted with EtOAc (20 mL). The organic layer was separated, dried over anhydrous Na₂SO₄ and concentrated under reduced pressure. After purified by prep-HPLC (column: Phenomenex C18 80×30 mm×5 μm; mobile phase: [water (NH₃H₂O+NH₄HCO₃)-ACN]; gradient: 46%-76% B over 7 min), 4-((1-(4-(2-(2-aminopyridin-3-yl)-5-(2-cyclopropyl-2H-1,2,3-triazol-4-yl)-3H-imidazo[4,5-h]pyridin-3-yl)benzyl)piperidin-4-yl)amino)pyrimidine-2-carbonitrile (Example 615, 68.7 mg, yield: 29% for three steps) was obtained as a yellow solid. MS: m/z=610.4 [M+H]⁺. ¹H NMR (400 MHz, Dimethylsulfoxide-d₆) δ 8.27 (d, J=8.4 Hz, 1H), 8.13-8.02 (m, 3H), 7.99 (dd, J=4.8, 1.6, Hz, 1H), 7.93 (d, J=8.4 Hz, 1H), 7.49-7.41 (m, 4H), 7.16-7.12 (m, 1H), 6.98 (s, 2H), 6.67 (d, J=6.0 Hz, 1H), 6.37 (dd, J=7.6, 4.8 Hz, 1H), 4.23-4.15 (m, 1H), 3.81-3.79 (m, 1H), 3.58 (s, 2H), 2.87-2.79 (m, 2H), 2.20-2.10 (m, 2H), 1.94-1.83 (m, 2H), 1.56-1.43 (m, 2H), 1.27-1.23 (m, 2H), 1.15-1.09 (m, 2H).

### Example 616: 4-((1-(4-(2-(2-Aminopyridin-3-yl)-5-(2-methyl-2H-1,2,3-triazol-4-yl-5-<)-3H-imidazo[4,5-b]pyridin-3- yl)benzyl)piperidin-4-yl)amino)pyrimidine-2-carbonitrile

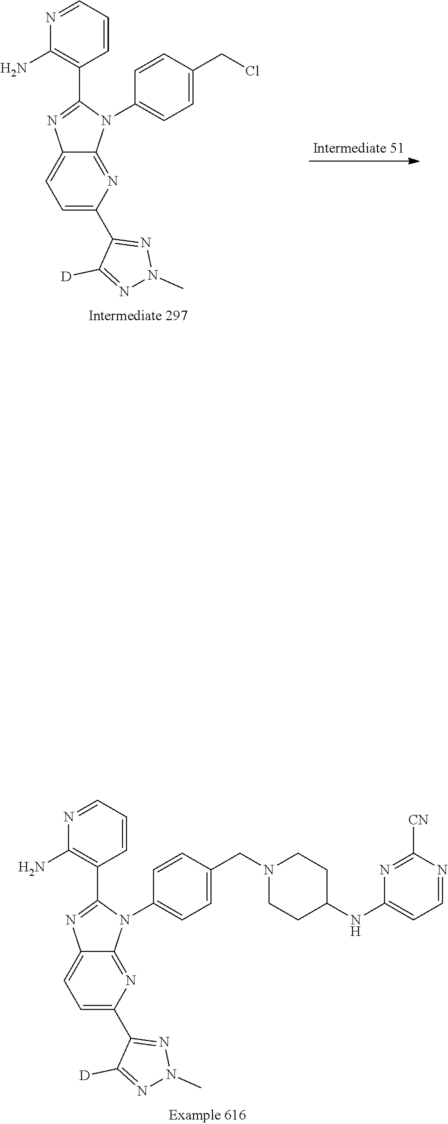 [SMILES: [2H]c1nn(C)nc1-c1ccc2nc(-c3cccnc3N)n(-c3ccc(CCl)cc3)c2n1 ; [2H]c1nn(C)nc1-c1ccc2nc(-c3cccnc3N)n(-c3ccc(CN4CCC(Nc5ccnc(C#N)n5)CC4)cc3)c2n1]

To a solution of Intermediate 297 (20 mg, 47.9 μmol) and 4-(piperidin-4-ylamino)pyrimidine-2-carbonitrile (15.2 mg, 47.9 μmol, TFA) in DMF (2 mL) were added K₂CO₃ (19.8 mg, 144 μmol) and NaI (717 μg, 4.79 μmol). The mixture was stirred at 50° C. for 1 hr. The reaction mixture was quenched with H₂O (5 mL) at 25° C. and extracted with CH₂Cl₂ (15 mL×2). The combined organic layers were washed with brine (50 mL×3), dried over Na₂SO₄, filtered and concentrated under reduced pressure. After purified by prep-TLC (SiO₂, CH₂Cl₂:MeOH=10:1), 4-((1-(4-(2-(2-aminopyridin-3-yl)-5-(2-methyl-2H-1,2,3-triazol-4-yl-5-d)-3H-imidazo[4,5-b]pyridin-3-yl)benzyl)piperidin-4-yl)amino)pyrimidine-2-carbonitrile (Example 616, 16.6 mg, yield: 58%) was obtained as a yellow solid. MS: m/z=585.2 [M+H]⁺. D %: 1D %=96.2%. ¹H NMR (400 MHz, Methanol-d₄) δ 8.17 (d, J=8.0 Hz, 1H), 8.04-7.95 (m, 3H), 7.53 (d, J=8.0 Hz, 2H), 7.42 (d, J=8.0 Hz, 2H), 7.31-7.23 (m, 1H), 6.60 (d, J=5.2 Hz, 1H), 6.45 (dd, J=7.6, 5.2 Hz, 1H), 4.21 (s, 3H), 4.04-3.89 (m, 1H), 3.67 (s, 2H), 3.02-2.92 (m, 2H), 2.38-2.22 (m, 2H), 2.04-1.97 (m, 2H), 1.68-1.56 (m, 2H).

### Example 617: 4-((1-(4-(2-(2-Aminopyridin-3-yl)-5-(1-(difluoromethyl)-6-oxo-1,6-dihydropyridin-3-yl)-3H-imidazo[4,5-b]pyridin-3-yl)benzyl)piperidin-4-yl)amino)pyrimidine-2-carbonitrile

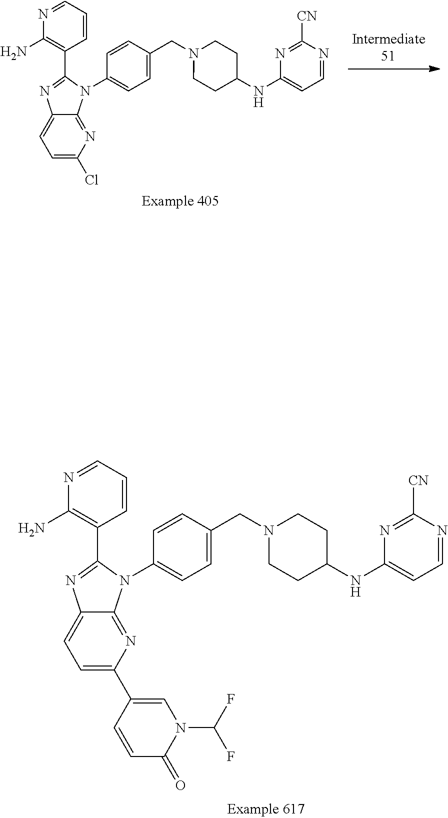 [SMILES: N#Cc1nccc(NC2CCN(Cc3ccc(-n4c(-c5cccnc5N)nc5ccc(Cl)nc54)cc3)CC2)n1 ; N#Cc1nccc(NC2CCN(Cc3ccc(-n4c(-c5cccnc5N)nc5ccc(-c6ccc(=O)n(C(F)F)c6)nc54)cc3)CC2)n1]

A mixture of Example 405 (119 mg, 222 μmol), Intermediate 299 (42 mg, 222 μmol), Cs₂CO₃ (217 mg, 667 μmol), and Pd(dppf)Cl₂ (16.3 mg, 22.2 μmol) in 1,4-dioxane (2 mL) and H₂O (0.4 mL) was degassed and purged with N₂ three times. The mixture was stirred at 80° C. for 2 hr under N₂ atmosphere. The reaction mixture was quenched with H₂O (20 mL) and extracted with CH₂Cl₂ (20 mL×2). The combined organic layers were washed with brine (20 mL), dried over Na₂SO₄, filtered and concentrated under reduced pressure. After purified by prep-HPLC (column: Waters Xbridge 150×25 mm×5 μm; mobile phase: [water (NH₄HCO₃)-ACN]; gradient: 33%-63% B over 12 min), 4-((1-(4-(2-(2-aminopyridin-3-yl)-5-(1-(difluoromethyl)-6-oxo-1,6-dihydropyridin-3-yl)-3H-imidazo[4,5-b]pyridin-3-yl)benzyl)piperidin-4-yl)amino)pyrimidine-2-carbonitrile (Example 617, 71.6 mg, yield: 49% for two steps) was obtained as a yellow lyophilized powder. MS: m/z=646.0 [M+H]⁺. ¹H NMR (400 MHz, Dimethylsulfoxide-d₆) δ 8.38-8.35 (m, 1H), 8.30-8.22 (m, 2H), 8.17-7.97 (m, 4H), 7.97-7.72 (m, 1H), 7.53-7.40 (m, 4H), 7.18-7.14 (m, 1H), 7.06 (s, 2H), 6.75-6.62 (m, 2H), 6.38 (dd, J=7.6, 4.8 Hz, 1H), 3.96-3.75 (m, 1H), 3.59 (s, 2H), 2.90-2.75 (m, 2H), 2.22-2.05 (m, 2H), 1.97-1.77 (m, 2H), 1.58-1.40 (m, 2H). ¹⁹F NMR (400 MHz, Dimethylsulfoxide-d₆) δ −103.343.

### Example 618: 4-((1-(4-(2-(2-Aminopyridin-3-yl)-5-(2-(fluoromethyl)-2H-1,2,3-triazol-4-yl)-3H-imidazo[4,5-b]pyridin-3-yl)benzyl)piperidin-4-yl)amino)pyrimidine-2-carbonitrile

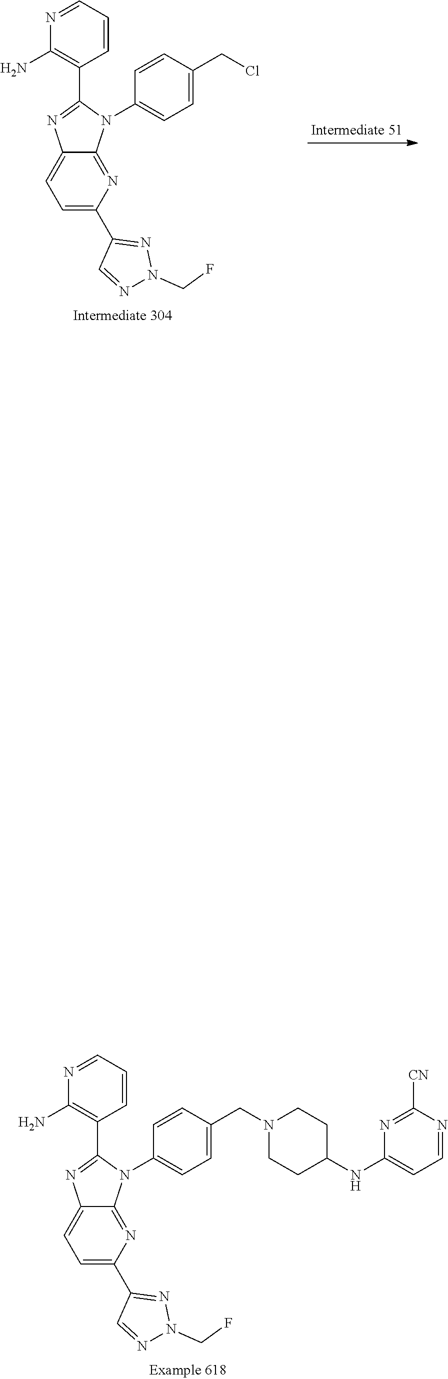 [SMILES: Nc1ncccc1-c1nc2ccc(-c3cnn(CF)n3)nc2n1-c1ccc(CCl)cc1 ; N#Cc1nccc(NC2CCN(Cc3ccc(-n4c(-c5cccnc5N)nc5ccc(-c6cnn(CF)n6)nc54)cc3)CC2)n1]

To a solution of Intermediate 304 (1.6 g, 3.39 mmol, HCl salt) and Intermediate 51 (1.08 g, 3.39 mmol, TFA) in DMF (30 mL) were added K₂CO₃ (1.41 g, 10.2 mmol) and NaI (35.2 mg, 235 μmol). The mixture was degassed, purged with N₂ three times, and stirred at 50° C. for 1 hr under N₂. The reaction mixture was diluted with CH₂Cl₂ (50 mL), washed with brine (30 mL×4), dried over Na₂SO₄, filtered, and concentrated under reduced pressure. After purified by silica gel flash chromatography (Eluent of 5%˜8% MeOH in CH₂Cl₂), 4-((1-(4-(2-(2-aminopyridin-3-yl)-5-(2-(fluoromethyl)-2H-1,2,3-triazol-4-yl)-3H-imidazo[4,5-b]pyridin-3-yl)benzyl)piperidin-4-yl)amino)pyrimidine-2-carbonitrile (Example 618, 1.01 g, yield 47% for three steps) was obtained as a yellow solid. MS: m/z=602.3 [M+H]⁺. ¹H NMR (400 MHz, Dimethylsulfoxide-d₆) δ 8.38-8.31 (m, 2H), 8.11-7.98 (m, 4H), 7.50-7.43 (m, 4H), 7.19-7.13 (m, 1H), 6.97 (br s, 2H), 6.68 (d, J=6.0 Hz, 1H), 6.48 (d, J=51.6 Hz, 2H), 6.38 (dd, J=7.6, 4.8 Hz, 1H), 3.88-3.75 (m, 1H), 3.59 (s, 2H), 2.86-2.81 (m, 2H), 2.20-2.12 (m, 2H), 1.94-1.85 (m, 2H), 1.55-1.45 (m, 2H). ¹⁹F NMR (400 MHz, Dimethylsulfoxide-d₆) δ −166.260.

### Example 619: 4-((1-(4-(2-(2-Aminopyridin-3-yl)-5-(1-(fluoromethyl)-1H-1,2,3-triazol-4-yl)-3H-imidazo[4,5-b]pyridin-3-yl)benzyl)piperidin-4-yl)amino)pyrimidine-2-carbonitrile

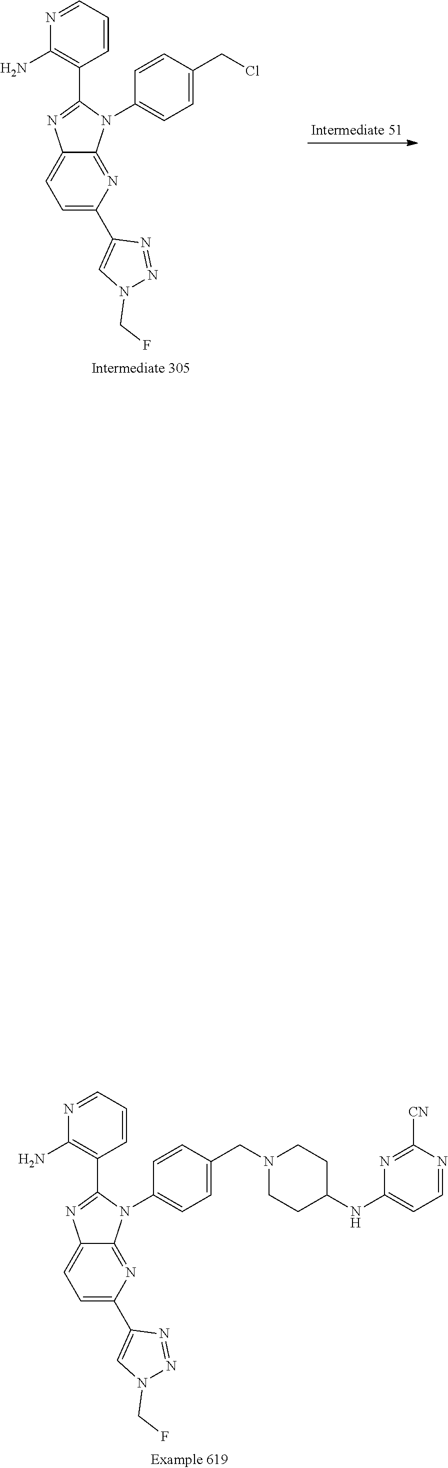 [SMILES: Nc1ncccc1-c1nc2ccc(-c3cn(CF)nn3)nc2n1-c1ccc(CCl)cc1 ; N#Cc1nccc(NC2CCN(Cc3ccc(-n4c(-c5cccnc5N)nc5ccc(-c6cn(CF)nn6)nc54)cc3)CC2)n1]

To a solution of Intermediate 305 (450 mg, 955 μmol, HCl salt) and Intermediate 51 (304 mg, 955 μmol, TFA) in DMF (30 mL) were added K₂CO₃ (396 mg, 2.86 mmol), NaI (28.6 mg, 191 μmol). The mixture was degassed and purged with N₂ for 3 times and stirred at 50° C. for 1 hr under N₂. The reaction mixture was filtered and concentrated under reduced pressure. After purified by prep-HPLC (column: Daisogel SP ODS RPS 150×25 mm×5 μm; mobile phase: [water (NH₄HCO₃)-ACN]; gradient: 25%-55% B over 11 min), 4-((1-(4-(2-(2-aminopyridin-3-yl)-5-(1-(fluoromethyl)-1H-1,2,3-triazol-4-yl)-3H-imidazo[4,5-b]pyridin-3-yl)benzyl)piperidin-4-yl)amino)pyrimidine-2-carbonitrile (Example 619, 175.1 mg, yield 30% for three steps) was obtained as a yellow lyophilized powder. MS: m/z=602.2 [M+H]⁺. ¹H NMR (400 MHz, Dimethylsulfoxide-d₆) δ 8.79 (s, 1H), 8.32 (d, J=8.4 Hz, 1H), 8.15-8.03 (m, 3H), 7.99 (dd, J=4.8, 1.6 Hz, 1H), 7.52-7.41 (m, 4H), 7.14 (dd, J=7.6, 1.6 Hz, 1H), 6.97 (br s, 2H), 6.68 (d, J=6.0 Hz, 1H), 6.48 (d, J=50.4 Hz, 2H), 6.37 (dd, J=7.6, 4.4 Hz, 1H), 3.84-3.80 (m, 1H), 3.58 (s, 2H), 2.86-2.81 (m, 2H), 2.21-2.10 (m, 2H), 1.92-1.87 (m, 2H), 1.59-1.43 (m, 2H). ¹⁹F NMR (400 MHz, Dimethylsulfoxide-d_{f}) 6-166.65.

### Intermediate 620: 4-((1-(4-(2-(2-Aminopyridin-3-yl)-5-(2-(difluoromethyl)-2H-1,2,3-triazol-4-yl)-3H-imidazo[4,5-b]pyridin-3-yl)benzyl)piperidin-4-yl)amino)pyrimidine-2-carbonitrile

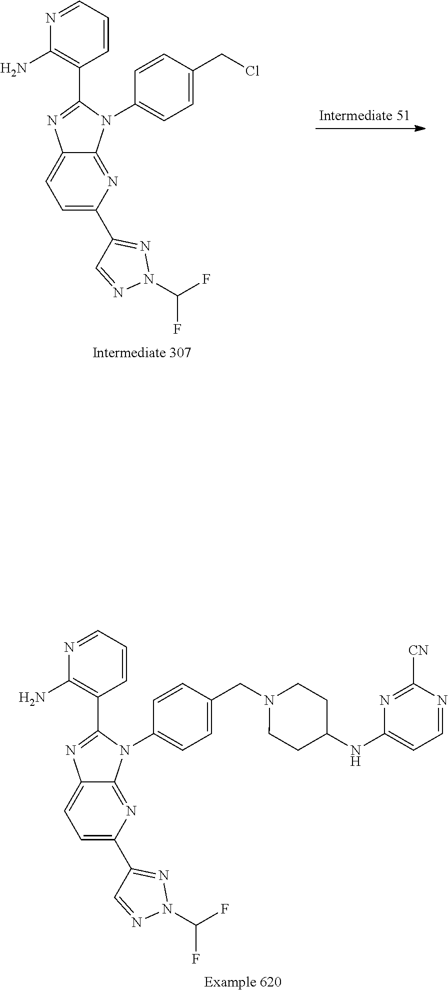 [SMILES: Nc1ncccc1-c1nc2ccc(-c3cnn(C(F)F)n3)nc2n1-c1ccc(CCl)cc1 ; N#Cc1nccc(NC2CCN(Cc3ccc(-n4c(-c5cccnc5N)nc5ccc(-c6cnn(C(F)F)n6)nc54)cc3)CC2)n1]

To a solution of Intermediate 307 (312 mg, 689 μmol, HCl salt) and Intermediate 51 (219 mg, 689 μmol, TFA) in DMF (4 mL) were added K₂CO₃ (381 mg, 2.76 mmol) and NaI (51.6 mg, 345 μmol). The mixture was stirred at 50° C. for 1 hr. The reaction mixture was quenched with H₂O (10 mL) at 25° C. and extracted with EtOAc (10 mL×3). The combined organic layers were washed with brine (50 mL×3), dried over Na₂SO₄, filtered and concentrated under reduced pressure. After purified by silica gel flash chromatography (Eluent of 0%˜5% CH₂Cl₂ in MeOH), 4-((1-(4-(2-(2-aminopyridin-3-yl)-5-(2-(difluoromethyl)-2H-1,2,3-triazol-4-yl)-3H-imidazo[4,5-b]pyridin-3-yl)benzyl)piperidin-4-yl)amino)pyrimidine-2-carbonitrile (Example 620, 202.9 mg, yield: 47% for three steps) was obtained as a yellow solid. MS: m/z=620.2 [M+H]⁺. ¹H NMR (400 MHz, Dimethylsulfoxide-d₆) δ 8.99 (s, 1H), 8.33 (d, J=8.0 Hz, 1H), 8.30-7.90 (m, 5H), 7.52-7.40 (m, 4H), 7.14 (d, J=6.8 Hz, 1H), 6.98 (br s, 2H), 6.68 (d, J=6.0 Hz, 1H), 6.37 (dd, J=7.2, 4.8 Hz, 1H), 3.91-3.72 (m, 1H), 3.58 (s, 2H), 2.91-2.74 (m, 2H), 2.24-2.09 (m, 2H), 1.96-1.78 (m, 2H), 1.57-1.42 (m, 2H). ¹⁹F NMR (400 MHz, Dimethylsulfoxide-d₆) δ −96.38.

### Example 621: 4-((1-(4-(2-(3-Aminopyrazin-2-yl)-5-(2-(methyl-d₃)-2H-1,2,3-triazol-4-yl)-3H-imidazo[4,5-b]pyridin-3-yl)benzyl)piperidin-4-yl)amino)pyrimidine-2-carbonitrile

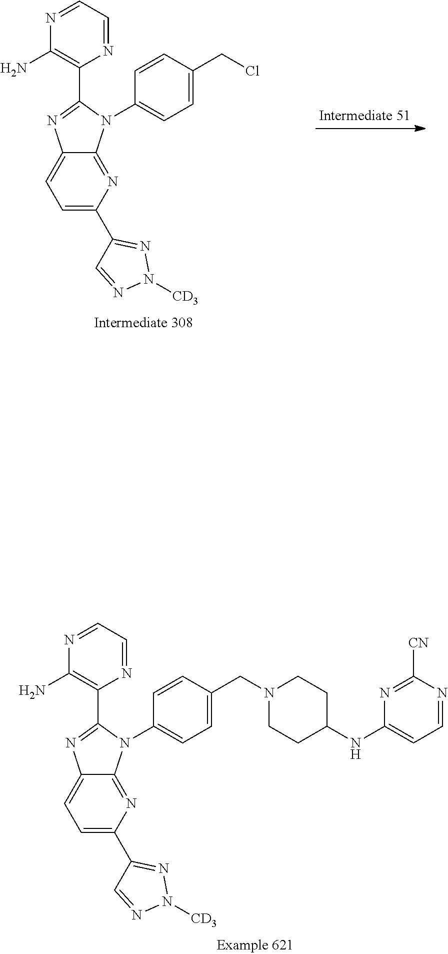 [SMILES: [2H]C([2H])([2H])n1ncc(-c2ccc3nc(-c4nccnc4N)n(-c4ccc(CCl)cc4)c3n2)n1 ; [2H]C([2H])([2H])n1ncc(-c2ccc3nc(-c4nccnc4N)n(-c4ccc(CN5CCC(Nc6ccnc(C#N)n6)CC5)cc4)c3n2)n1]

To a solution of Intermediate 308 (220 mg, 481 μmol, HCl salt) and Intermediate 51 (153 mg, 481 μmol, TFA) in DMF (3 mL) were added K₂CO₃ (332 mg, 2.41 mmol) and NaI (7.21 mg, 48.1 μmol). The mixture was stirred at 50° C. for 1 hr. The reaction mixture was quenched with H₂O (5 mL) at 25° C. and extracted with CH₂Cl₂ (15 mL×2). The combined organic layers were washed with brine (50 mL×3), dried over Na₂SO₄, filtered and concentrated under reduced pressure. After purified by prep-HPLC (column: Waters Xbridge BEH C18 150×25 mm×5 μm; mobile phase: [water (NH₄HCO₃)-ACN]; gradient: 32%-62% B over 10 min), 4-((1-(4-(2-(3-aminopyrazin-2-yl)-5-(2-(methyl-d₃)-2H-1,2,3-triazol-4-yl)-3H-imidazo[4,5-b]pyridin-3-yl)benzyl)piperidin-4-yl)amino)pyrimidine-2-carbonitrile (Example 621, 57.8 mg, yield: 20% for two steps) was obtained as a yellow lyophilized powder. MS: m/z=588.1 [M+H]⁺. D %: 3D %=99.1%. ¹H NMR (400 MHz, Dimethylsulfoxide-d₆) δ 8.35 (d, J=8.4 Hz, 1H), 8.24-7.86 (m, 5H), 7.77 (br s, 2H), 7.51-7.30 (m, 5H), 6.68 (d, J=5.6 Hz, 1H), 3.91-3.77 (m, 1H), 3.59 (s, 2H), 2.91-2.81 (m, 2H), 2.24-2.11 (m, 2H), 2.00-1.79 (m, 2H), 1.59-1.41 (m, 2H).

### Example 622: 4-((1-(4-(2-(3-Aminoisoxazol-4-yl)-5-(5-fluoropyridin-2-yl)-3H-imidazo[4,5-b]pyridin-3-yl)benzyl)piperidin-4-yl)amino)pyrimidine-2-carbonitrile

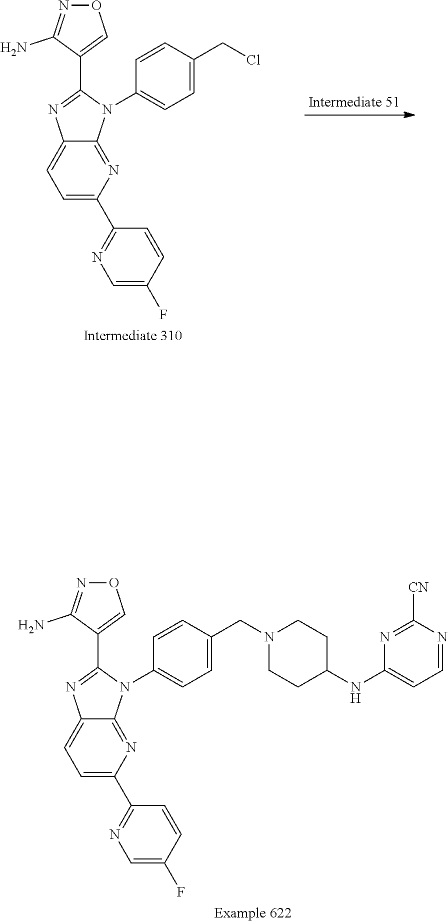 [SMILES: Nc1nocc1-c1nc2ccc(-c3ccc(F)cn3)nc2n1-c1ccc(CCl)cc1 ; N#Cc1nccc(NC2CCN(Cc3ccc(-n4c(-c5conc5N)nc5ccc(-c6ccc(F)cn6)nc54)cc3)CC2)n1]

To a solution of Intermediate 310 (58.7 mg, 128 μmol, HCl salt), Intermediate 51 (40.7 mg, 128 μmol, TFA salt) in DMF (1 mL) were added K₂CO₃ (70.9 mg, 513 μmol) and NaI (19.2 mg, 128 μmol). The mixture was degassed and purged with N₂ for 3 times and stirred at 25° C. for 1 hr under N₂. The reaction mixture was quenched with H₂O (10 mL) and extracted with CH₂Cl₂ (5 mL×3). The combined organic layers were washed with brine (10 mL×2), dried over Na₂SO₄, filtered and concentrated under reduced pressure. After purified by silica gel flash chromatography (Eluent of 0% ˜9% MeOH in CH₂Cl₂), 4-((1-(4-(2-(3-aminoisoxazol-4-yl)-5-(5-fluoropyridin-2-yl)-3H-imidazo[4,5-b]pyridin-3-yl)benzyl)piperidin-4-yl)amino)pyrimidine-2-carbonitrile (Example 622, 22.6 mg, yield: 29% for three steps) was obtained as a yellow powder. MS: m/z=588.4 [M+H]⁺. ¹H NMR (400 MHz, Dimethylsulfoxide-d₆) δ 8.66 (d, J=2.8 Hz, 1H), 8.41-8.36 (m, 1H), 8.33-8.29 (m, 1H), 8.15 (dd, J=9.2, 4.8 Hz, 1H), 8.12-8.06 (m, 2H), 7.80-7.78 (m, 1H), 7.65-7.59 (m, 4H), 7.52 (s, 1H), 6.71-6.64 (m, 3H), 3.91-3.77 (m, 1H), 3.69 (s, 2H), 2.96-2.88 (m, 2H), 2.28-2.18 (m, 2H), 1.97-1.88 (m, 2H), 1.59-1.50 (m, 2H). ¹⁹F NMR (400 MHz, Dimethylsulfoxide-d₆) δ −127.739.

### Example 623: 4-((1-(4-(2-(2-Aminopyridin-3-yl)-5-(6-(fluoromethoxy-d₂)pyridin-3-yl)-3H-imidazo[4,5-b]pyridin-3-yl)benzyl)piperidin-4-yl)amino)pyrimidine-2-carbonitrile

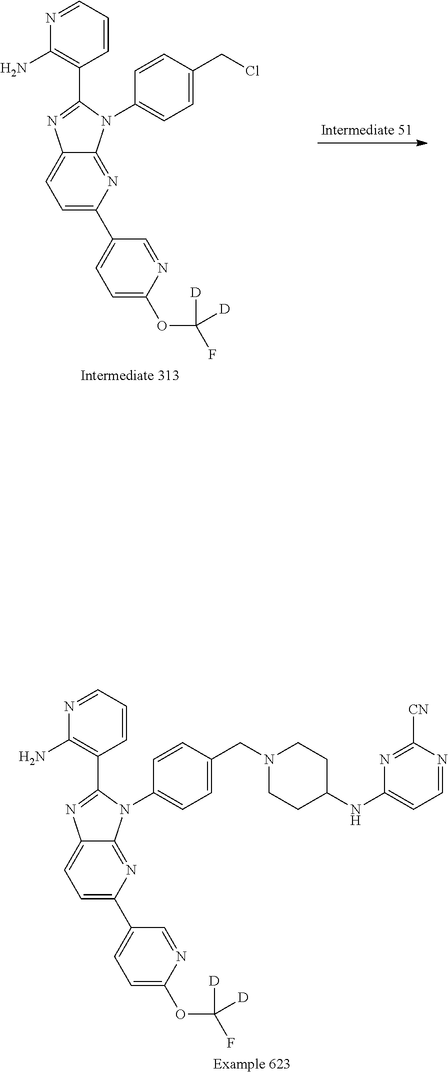 [SMILES: [2H]C([2H])(F)Oc1ccc(-c2ccc3nc(-c4cccnc4N)n(-c4ccc(CCl)cc4)c3n2)cn1 ; [2H]C([2H])(F)Oc1ccc(-c2ccc3nc(-c4cccnc4N)n(-c4ccc(CN5CCC(Nc6ccnc(C#N)n6)CC5)cc4)c3n2)cn1]

To a solution of Intermediate 313 (300 mg, 601 μmol, HCl salt) and Intermediate 51 (191 mg, 601 μmol, TFA) in DMF (6 mL) were added K₂CO₃ (249 mg, 1.80 mmol) and NaI (18.0 mg, 120 μmol). The mixture was stirred at 50° C. for 1 hr. The reaction mixture was quenched with H₂O (10 mL) at 25° C. and extracted with CH₂Cl₂ (10 mL×3). The combined organic layers were washed with brine (20 mL×3), dried over Na₂SO₄, filtered and concentrated under reduced pressure. After purified by silica gel flash chromatography (Eluent of 5%˜8% MeOH in CH₂Cl₂) and triturated with EtOAc (10 mL) at 25° C. for 5 min., 4-((1-(4-(2-(2-aminopyridin-3-yl)-5-(6-(fluoromethoxy-d₂)pyridin-3-yl)-3H-imidazo[4,5-b]pyridin-3-yl)benzyl)piperidin-4-yl)amino)pyrimidine-2-carbonitrile (Intermediate 623, 86.5 mg, yield: 23% for two steps) was obtained as an off-white solid. MS: m/z=630.2 [M+H]⁺. D %: 2D %=98.2%. ¹H NMR (400 MHz, Dimethylsulfoxide-d₆) δ 8.90 (d, J=2.4 Hz, 1H), 8.42 (dd, J=8.4, 2.4 Hz, 1H), 8.29 (d, J=8.4 Hz, 1H), 8.09-7.99 (m, 4H), 7.50-7.43 (m, 4H), 7.18-7.13 (m, 1H), 7.09 (d, J=8.8 Hz, 1H), 7.01 (br s, 2H), 6.67 (br d, J=5.2 Hz, 1H), 6.39 (dd, J=7.6, 4.8 Hz, 1H), 3.84-3.79 (m, 1H), 3.59 (br s, 2H), 2.85-2.79 (m, 2H), 2.19-2.11 (m, 2H), 1.93-1.83 (m, 2H), 1.55-1.46 (m, 2H). ¹⁹F NMR (400 MHz, Dimethylsulfoxide-d₆) δ −155.547.

### Example 624: 4-((1-(4-(2-(2-Aminopyridin-3-yl)-5-(1-(fluoromethyl-d₂)-6-oxo-1,6-dihydropyridin-3-yl)-3H-imidazo[4,5-b]pyridin-3-yl)benzyl)piperidin-4-yl)amino)pyrimidine-2-carbonitrile

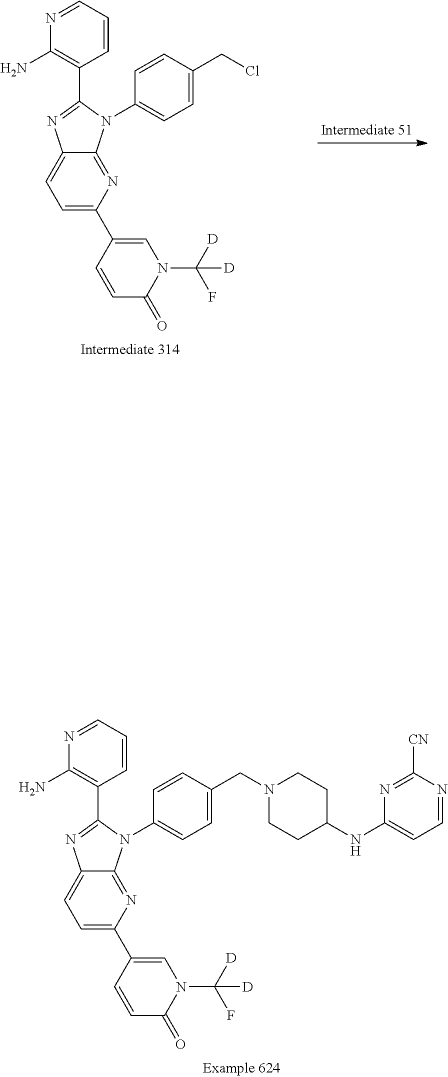 [SMILES: [2H]C([2H])(F)n1cc(-c2ccc3nc(-c4cccnc4N)n(-c4ccc(CCl)cc4)c3n2)ccc1=O ; [2H]C([2H])(F)n1cc(-c2ccc3nc(-c4cccnc4N)n(-c4ccc(CN5CCC(Nc6ccnc(C#N)n6)CC5)cc4)c3n2)ccc1=O]

To a solution of Intermediate 314 (101 mg, 202 μmol, HCl salt) and Intermediate 51 (64.2 mg, 202 μmol, TFA) in DMF (3 mL) were added K₂CO₃ (140 mg, 1.01 mmol) and NaI (3.03 mg, 20.2 μmol). The mixture was stirred at 50° C. for 1 hr. The reaction mixture was quenched with H₂O (5 mL) at 25° C. and extracted with CH₂Cl₂ (15 mL×2). The combined organic layers were washed with brine (20 mL×3), dried over Na₂SO₄, filtered and concentrated under reduced pressure. After purified by silica gel flash chromatography (Eluent of 1%˜8% MeOH in CH₂Cl₂), 4-((1-(4-(2-(2-aminopyridin-3-yl)-5-(1-(fluoromethyl-d₂)-6-oxo-1,6-dihydropyridin-3-yl)-3H-imidazo[4,5-b]pyridin-3-yl)benzyl)piperidin-4-yl)amino)pyrimidine-2-carbonitrile (Example 624, 37.1 mg, yield: 28% for two steps) was obtained as a yellow solid. MS: m/z=630.2 [M+H]⁺. D %: 2D %=98.4%. ¹H NMR (400 MHz, Dimethylsulfoxide-d₆) δ 8.60 (d, J=2.8 Hz, 1H), 8.27 (d, J=8.4 Hz, 1H), 8.18 (dd, J=9.6, 2.4 Hz, 1H), 8.14-8.02 (m, 2H), 8.01-7.96 (m, 1H), 7.86 (d, J=8.4 Hz, 1H), 7.51-7.40 (m, 4H), 7.16-7.10 (m, 1H), 7.02 (br s, 2H), 6.67 (d, J=6.0 Hz, 1H), 6.61 (d, J=9.6 Hz, 1H), 6.37 (dd, J=7.6, 4.8 Hz, 1H), 3.85-3.74 (m, 1H), 3.59 (s, 2H), 2.91-2.75 (m, 2H), 2.18-2.09 (m, 2H), 1.95-1.84 (m, 2H), 1.55-1.44 (m, 2H). ¹⁹F NMR (400 MHz, Dimethylsulfoxide-d₆) δ −173.761.

### Example 625: 4-((1-(4-(2-(2-Aminopyridin-3-yl)-5-(1-ethyl-1H-1,2,3-triazol-4-yl)-3H-imidazo[4,5-b]pyridin-3-yl)benzyl)piperidin-4-yl)amino)pyrimidine-2-carbonitrile

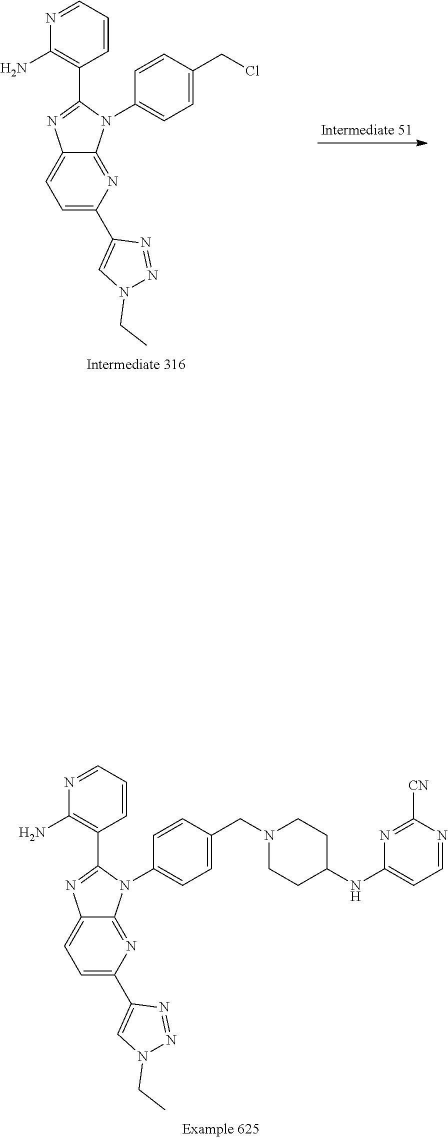 [SMILES: CCn1cc(-c2ccc3nc(-c4cccnc4N)n(-c4ccc(CCl)cc4)c3n2)nn1 ; CCn1cc(-c2ccc3nc(-c4cccnc4N)n(-c4ccc(CN5CCC(Nc6ccnc(C#N)n6)CC5)cc4)c3n2)nn1]

To a mixture of Intermediate 316 (110 mg, 255 μmol) and Intermediate 51 (57.1 mg, 280 μmol) in DMF (2 mL) were added K₂CO₃ (106 mg, 766 μmol) and NaI (3.83 mg, 25.5 μmol), the mixture was stirred at 20° C. for 12 hr. The mixture was diluted with H₂O (20 mL) and extracted with EtOAc (10 mL×2). The combined organic layers were washed with brine (10 mL), dried over anhydrous Na₂SO₄, filtered and concentrated. After purified by prep-HPLC (column: C18 150×30 mm; mobile phase: [water(NH₃H₂O+NH₄HCO₃)-ACN]; gradient: 45%-75% B over 7 min), 4-((1-(4-(2-(2-aminopyridin-3-yl)-5-(1-ethyl-1H-1,2,3-triazol-4-yl)-3H-imidazo[4,5-b]pyridin-3-yl)benzyl)piperidin-4-yl)amino)pyrimidine-2-carbonitrile (Example 625, 48.8 mg, yield: 32%) was obtained as a white solid. MS: m/z=598.2 [M+H]⁺. ¹H NMR (400 MHz, Dimethylsulfoxide-d₆) δ 8.41 (s, 1H), 8.27 (d, J=8.4 Hz, 1H), 8.12-8.04 (m, 3H), 7.99 (dd, J=4.8, 1.6 Hz, 1H), 7.50-7.40 (m, 4H), 7.11 (dd, J=8.0, 2.0 Hz, 1H), 6.97 (s, 2H), 6.68 (d, J=6.0 Hz, 1H), 6.36 (dd, J=7.6, 4.8 Hz, 1H), 4.42 (q, J=7.2 Hz, 2H), 3.89-3.77 (m, 1H), 3.59 (s, 2H), 2.93-2.79 (m, 2H), 2.25-2.09 (m, 2H), 1.97-1.84 (m, 2H), 1.54-1.42 (m, 5H).

### Example 626: 4-((1-(4-(2-(2-Aminopyridin-3-yl)-5-(2-(cyanomethyl)-2H-1,2,3-triazol-4-yl)-3H-imidazo[4,5-b]pyridin-3-yl)benzyl)piperidin- 4-yl)amino)pyrimidine-2-carbonitrile

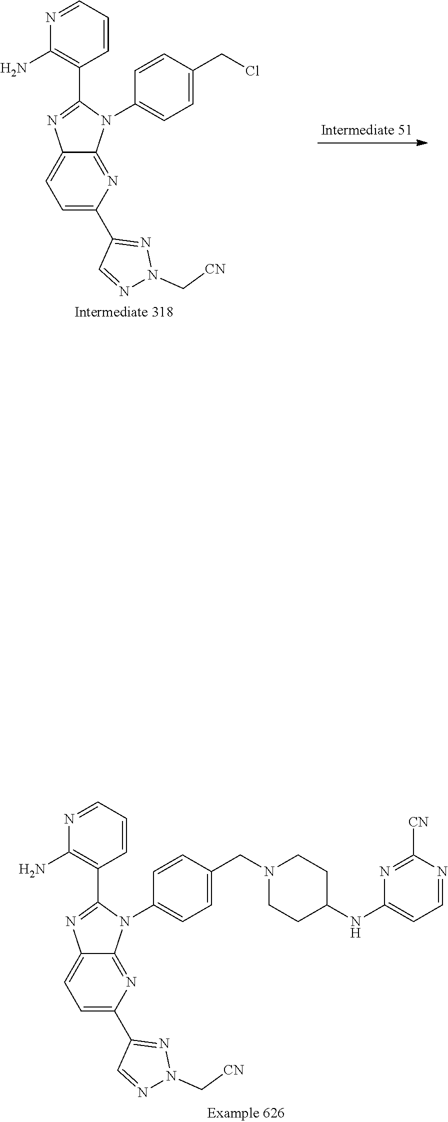 [SMILES: N#CCn1ncc(-c2ccc3nc(-c4cccnc4N)n(-c4ccc(CCl)cc4)c3n2)n1 ; N#CCn1ncc(-c2ccc3nc(-c4cccnc4N)n(-c4ccc(CN5CCC(Nc6ccnc(C#N)n6)CC5)cc4)c3n2)n1]

To a solution of Intermediate 318 (90 mg, 204 μmol) and Intermediate 51 (53.8 mg, 265 μmol) in DMF (1 mL) were added NaI (3.05 mg, 20.4 μmol) and K₂CO₃ (84.5 mg, 611 μmol). The mixture was stirred at 25° C. for 12 hr under N₂. The reaction mixture was quenched with H₂O (10 mL) at 25° C. and extracted with CH₂Cl₂ (10 mL×3). The combined organic layers were washed with brine (20 mL×3), dried over Na₂SO₄, filtered and concentrated under reduced pressure. After purified by prep-HPLC (column: Phenomenex C18 80×30 mm×5 μm; mobile phase: [water (NH₃H₂O+NH₄HCO₃)-ACN]; gradient: 45%-75% B over 7 min), 4-((1-(4-(2-(2-aminopyridin-3-yl)-5-(2-(cyanomethyl)-2H-1,2,3-triazol-4-yl)-3H-imidazo[4,5-b]pyridin-3-yl)benzyl)piperidin-4-yl)amino)pyrimidine-2-carbonitrile (Example 626, 21.6 mg, yield: 17% for three steps) was obtained as an off-white solid. MS: m/z=609.4 [M+H]⁺. ¹H NMR (400 MHz, Dimethylsulfoxide-d₆) δ 8.32 (d, J=8.4 Hz, 1H), 8.28 (s, 1H), 8.09 (d, J=5.6 Hz, 2H), 8.02-7.95 (m, 2H), 7.51-7.39 (m, 4H), 7.15 (dd, J=7.6, 1.2 Hz, 1H), 6.97 (s, 2H), 6.68 (d, J=6.0 Hz, 1H), 6.38 (dd, J=8.0, 5.2 Hz, 1H), 5.99 (s, 2H), 3.95-3.75 (m, 1H), 3.59 (br s, 2H), 2.97-2.69 (m, 2H), 2.28-2.03 (m, 2H), 2.01-1.82 (m, 2H), 1.59-1.39 (m, 2H).

### Example 627: 4-((1-(4-(2-(3-Aminopyrazin-2-yl)-5-(2-(fluoromethyl)-2H-1,2,3-triazol-4-yl)-3H-imidazo[4,5-b]pyridin-3-yl)benzyl)piperidin-4-yl)amino)pyrimidine-2-carbonitrile

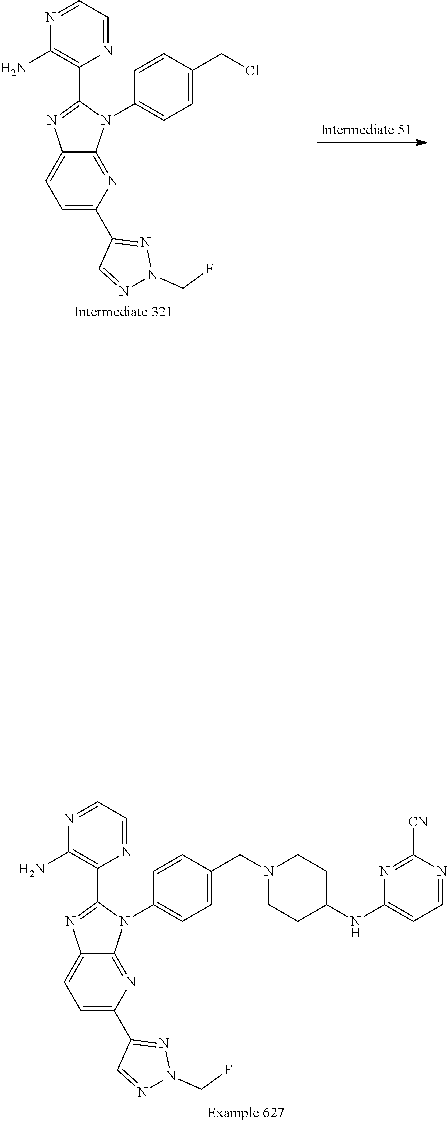 [SMILES: Nc1nccnc1-c1nc2ccc(-c3cnn(CF)n3)nc2n1-c1ccc(CCl)cc1 ; N#Cc1nccc(NC2CCN(Cc3ccc(-n4c(-c5nccnc5N)nc5ccc(-c6cnn(CF)n6)nc54)cc3)CC2)n1]

To a solution of Intermediate 321 (200 mg, 423 μmol, HCl salt) and Intermediate 51 (135 mg, 423 μmol, TFA) in DMF (3 mL) were added K₂CO₃ (176 mg, 1.27 mmol) and NaI (12.7 mg, 84.7 μmol). The mixture was degassed and purged with N₂ three times and stirred at 50° C. for 1 hr under N₂. The reaction mixture was filtered and concentrated under reduced pressure. After purified by prep-HPLC (column: Daisogel SP ODS RPS 150×25 mm×5 sm; mobile phase: [water (NH₄HCO₃)-ACN]; gradient: 35%-65% B over 11 min), 4-((1-(4-(2-(3-aminopyrazin-2-yl)-5-(2-(fluoromethyl)-2H-1,2,3-triazol-4-yl)-3H-imidazo[4,5-b]pyridin-3-yl)benzyl)piperidin-4-yl)amino)pyrimidine-2-carbonitrile (Example 627, 24.3 mg, yield 9.3% for three steps) was obtained as a gray solid. MS: m/z=603.2 [M+H]⁺. ¹H NMR (400 MHz, Dimethylsulfoxide-d₆) δ 8.41 (d, J=8.4 Hz, 1H), 8.33 (s, 1H), 8.12-7.99 (m, 4H), 7.77 (br s, 2H), 7.49 (d, J=2.4 Hz, 1H), 7.46-7.41 (m, 2H), 7.40-7.36 (m, 2H), 6.68 (d, J=6.8 Hz, 1H), 6.48 (d, J=51.6 Hz, 2H), 3.87-3.79 (m, 1H), 3.59 (s, 2H), 2.88-2.83 (m, 2H), 2.21-2.14 (m, 2H), 1.96-1.88 (m, 2H), 1.57-1.47 (m, 2H). ¹⁹F NMR (400 MHz, Dimethylsulfoxide-d₆) δ −166.381.

### Example 628: 4-((1-(4-(2-(3-Aminopyrazin-2-yl)-5-(1-(fluoromethyl)-1H-1,2,3-triazol-4-yl)-3H-imidazo[4,5-b]pyridin-3-yl)benzyl)piperidin-4-yl)amino)pyrimidine-2-carbonitrile

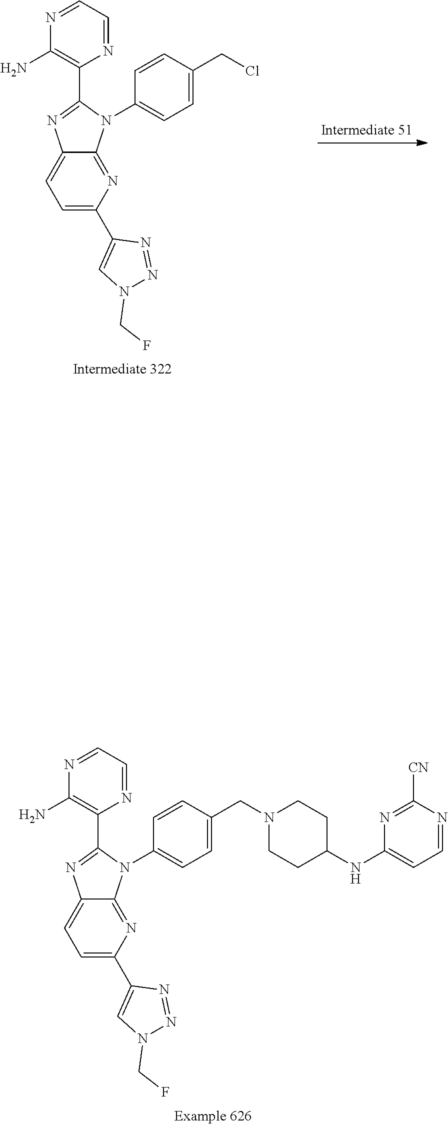 [SMILES: Nc1nccnc1-c1nc2ccc(-c3cn(CF)nn3)nc2n1-c1ccc(CCl)cc1 ; N#Cc1nccc(NC2CCN(Cc3ccc(-n4c(-c5nccnc5N)nc5ccc(-c6cn(CF)nn6)nc54)cc3)CC2)n1]

To a solution of Intermediate 322 (70 mg, 148 μmol, HCl salt) and Intermediate 51 (47.3 mg, 148 μmol, TFA) in DMF (2 mL) were added K₂CO₃ (61.5 mg, 445 μmol), NaI (4.44 mg, 29.6 μmol). The mixture was degassed and purged with N₂ three times and stirred at 50° C. for 1 hr under N₂. The reaction mixture was filtered and concentrated under reduced pressure. After purified by prep-HPLC (column: Daisogel SP ODS RPS 150×25 mm×5 μm; mobile phase: [water (NH₄HCO₃)-ACN]; gradient: 28%-58% B over 11 min), 4-((1-(4-(2-(3-aminopyrazin-2-yl)-5-(1-(fluoromethyl)-1H-1,2,3-triazol-4-yl)-3H-imidazo[4,5-b]pyridin-3-yl)benzyl)piperidin-4-yl)amino)pyrimidine-2-carbonitrile (Example 628, 14.2 mg, yield 15% for three steps) was obtained as a yellow solid. MS: m/z=603.3 [M+H]⁺. ¹H NMR (400 MHz, Methanol-d₄) δ 8.58 (s, 1H), 8.30 (d, J=8.4 Hz, 1H), 8.19 (d, J=8.4 Hz, 1H), 8.04-7.98 (m, 1H), 7.94 (d, J=2.0 Hz, 1H), 7.52-7.48 (m, 3H), 7.38-7.34 (m, 2H), 6.64-6.58 (m, 1H), 6.38 (d, J=50.8 Hz, 2H), 4.05-3.92 (m, 1H), 3.69 (s, 2H), 3.04-3.00 (m, 2H), 2.35-2.26 (m, 2H), 2.07-2.01 (m, 2H), 1.70-1.60 (m, 2H). ¹⁹F NMR (400 MHz, Methanol-d₄) δ −169.788.

### Example 629: 4-((1-(4-(2-(3-Aminopyrazin-2-yl)-5-(2-ethyl-2H-1,2,3-triazol-4-yl)-3H-imidazo[4,5-b]pyridin-3-yl)benzyl)piperidin-4-yl)amino)pyrimidine-2-carbonitrile

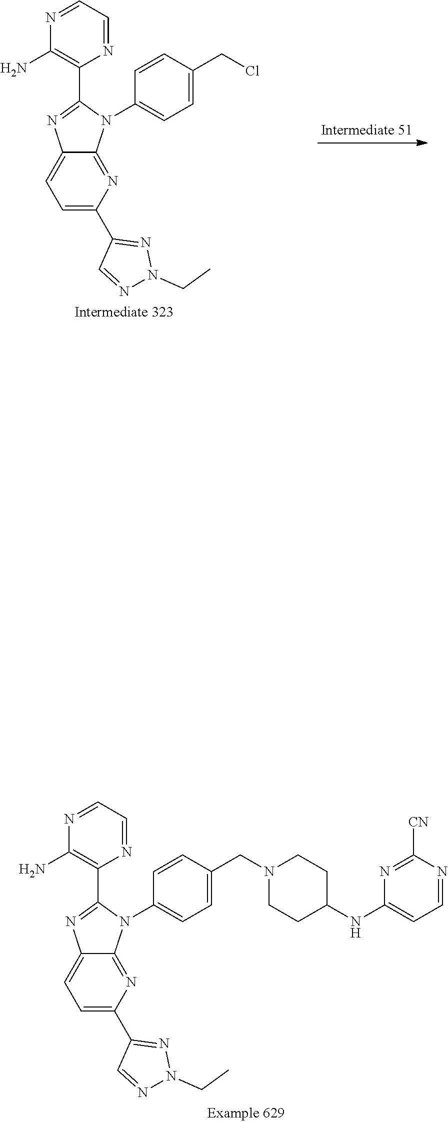 [SMILES: CCn1ncc(-c2ccc3nc(-c4nccnc4N)n(-c4ccc(CCl)cc4)c3n2)n1 ; CCn1ncc(-c2ccc3nc(-c4nccnc4N)n(-c4ccc(CN5CCC(Nc6ccnc(C#N)n6)CC5)cc4)c3n2)n1]

To a mixture of Intermediate 323 (190 mg, 440 μmol) and Intermediate 51 (98.4 mg, 484 mol) in DMF (3 mL) were added NaI (6.59 mg, 44.0 μmol) and K₂CO₃ (182 mg, 1.32 mmol). The mixture was stirred at 20° C. for 2 hr. The reaction mixture was quenched with H₂O (50 mL) and extracted with EtOAc (50 mL×2). The combined organic layers were washed with brine (50 mL), dried over anhydrous Na₂SO₄, filtered and concentrated. After purified by prep-HPLC (column: C18 150×30 mm; mobile phase: [water(NH₃H₂O+NH₄HCO₃)-ACN]; gradient: 48%-78% B over 7 min), 4-((1-(4-(2-(3-aminopyrazin-2-yl)-5-(2-ethyl-2H-1,2,3-triazol-4-yl)-3H-imidazo[4,5-b]pyridin-3-yl)benzyl)piperidin-4-yl)amino)pyrimidine-2-carbonitrile (Example 629, 107 mg, yield: 40% for two steps) was obtained as a yellow solid. MS: m/z=621.1 [M+Na]⁺. ¹H NMR (400 MHz, Dimethylsulfoxide-d₆) δ 8.33 (d, J=8.4 Hz, 1H), 8.12-7.93 (m, 5H), 7.78 (br s, 2H), 7.47 (d, J=2.4 Hz, 1H), 7.45-7.33 (m, 4H), 6.68 (d, J=6.0 Hz, 1H), 4.50 (q, J=7.2 Hz, 2H), 3.89-3.75 (m, 1H), 3.59 (s, 2H), 2.96-2.80 (m, 2H), 2.24-2.10 (m, 2H), 1.98-1.82 (m, 2H), 1.57-1.41 (m, 5H).

### Example 630: 4-((1-(4-(2-(3-Aminopyrazin-2-yl)-5-(2-isopropyl-2H-1,2,3-triazol-4-yl)-3H-imidazo[4,5-b]pyridin-3-yl)benzyl)piperidin-4-yl)amino)pyrimidine-2-carbonitrile

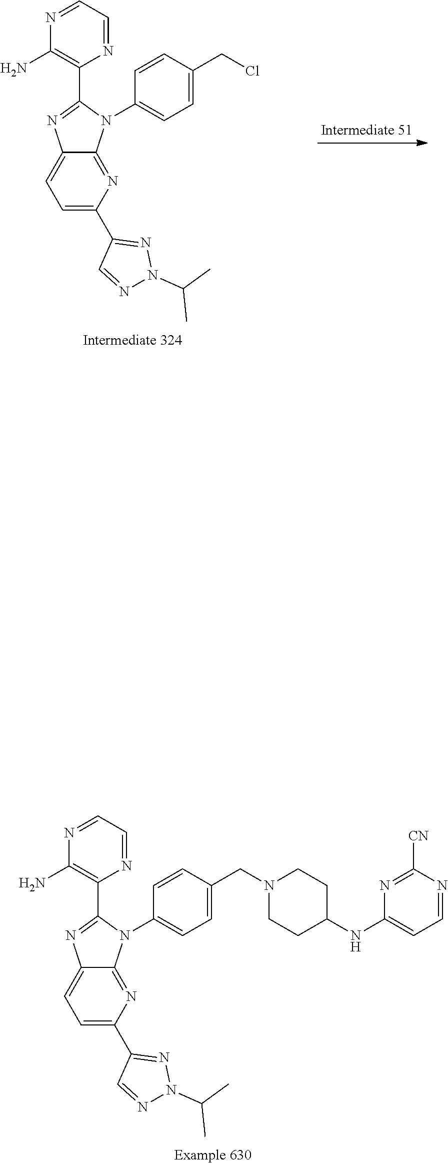 [SMILES: CC(C)n1ncc(-c2ccc3nc(-c4nccnc4N)n(-c4ccc(CCl)cc4)c3n2)n1 ; CC(C)n1ncc(-c2ccc3nc(-c4nccnc4N)n(-c4ccc(CN5CCC(Nc6ccnc(C#N)n6)CC5)cc4)c3n2)n1]

To a solution of Intermediate 324 (187 mg, 419 μmol) and Intermediate 51 (93.8 mg, 461 μmol) in DMF (8 mL) were added K₂CO₃ (174 mg, 1.26 mmol) and NaI (12.6 mg, 84 μmol) at 20° C. The reaction mixture was stirred at 20° C. for 12 hr. The mixture was diluted with H₂O (20 mL) and extracted with EtOAc (20 mL×2). The combined organic layers were washed with brine (20 mL), dried over anhydrous Na₂SO₄, filtered and concentrated. After purified by prep-HPLC (column: Xtimate C18 150×40 mm×10 μm; mobile phase: [water(FA)-ACN]; gradient: 25%-55% B over 7 min), the fractions were concentrated to remove most of MeCN. The product was diluted with CH₂Cl₂ (30 mL) and washed with sat. NaHCO₃ (30 mL×2). The organic layer was dried over anhydrous Na₂SO₄, filtered and concentrated to give 4-((1-(4-(2-(3-aminopyrazin-2-yl)-5-(2-isopropyl-2H-1,2,3-triazol-4-yl)-3H-imidazo[4,5-b]pyridin-3-yl)benzyl)piperidin-4-yl)amino)pyrimidine-2-carbonitrile (Example 630, 61.0 mg, yield: 23.6% for 3 steps) as a yellow solid. MS: m/z=613.3 [M+H]⁺. ¹H NMR (400 MHz, Dimethylsulfoxide-d₆) δ 8.37-8.32 (m, 1H), 8.12-7.96 (m, 5H), 7.78 (s, 2H), 7.48-7.34 (m, 5H), 6.70-6.64 (m, 1H), 4.91-4.84 (m, 1H), 3.88-3.76 (m, 1H), 3.59 (s, 2H), 2.91-2.80 (m, 2H), 2.20-2.11 (m, 2H), 1.96-1.85 (m, 2H), 1.56-1.48 (m, 8H).

### Example 631: 4-((1-(4-(2-(3-Aminopyrazin-2-yl)-5-(2-cyclopropyl-2H-1,2,3-triazol-4-yl)-3H-imidazo[4,5-b]pyridin-3-yl)benzyl)piperidin-4-yl)amino)pyrimidine-2-carbonitrile

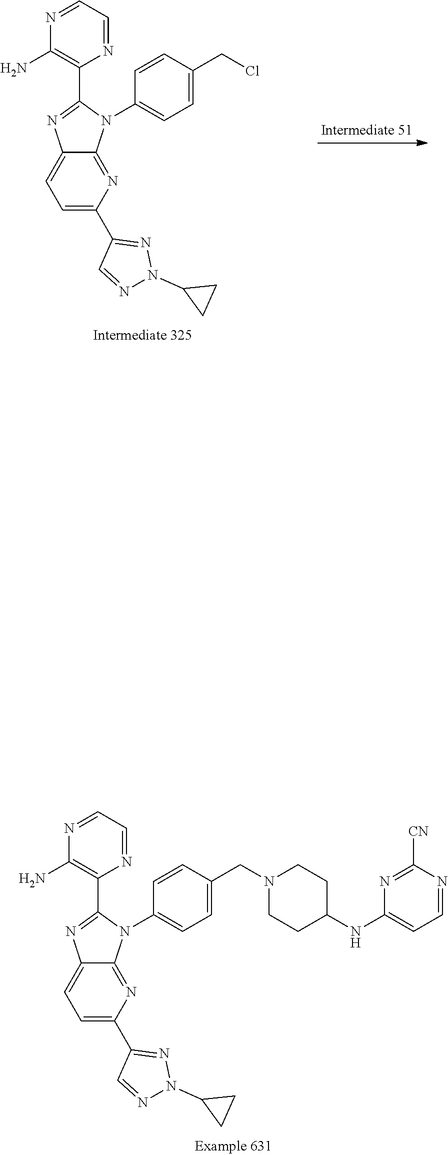 [SMILES: Nc1nccnc1-c1nc2ccc(-c3cnn(C4CC4)n3)nc2n1-c1ccc(CCl)cc1 ; N#Cc1nccc(NC2CCN(Cc3ccc(-n4c(-c5nccnc5N)nc5ccc(-c6cnn(C7CC7)n6)nc54)cc3)CC2)n1]

To a solution of Intermediate 325 (130 mg, 642 μmol, HCl salt) and Intermediate 51 (130 mg, 642 μmol) in DMF (3 mL) were added NaI (8.02 mg, 53.5 μmol) and K₂CO₃ (222 mg, 1.60 mmol). The mixture was stirred at 25° C. for 1 hr. The mixture was diluted with water (10 mL) and extracted with ethyl acetate (10 mL×2). The combined organic layers were dried over anhydrous Na₂SO₄, filtered and concentrated. After purified by prep-HPLC (column: C18 150×30 mm; mobile phase: [water (NH₃H₂O+NH₄HCO₃)-ACN]; gradient: 55%-85% B over 7 min), 4-((1-(4-(2-(3-aminopyrazin-2-yl)-5-(2-cyclopropyl-2H-1,2,3-triazol-4-yl)-3H-imidazo[4,5-b]pyridin-3-yl)benzyl)piperidin-4-yl)amino)pyrimidine-2-carbonitrile (Example 631, 60.8 mg, yield: 19% for three steps) was obtained as a light yellow solid. MS: m/z=611.1 [M+H]⁺. ¹H NMR (400 MHz, Dimethylsulfoxide-d₆) δ 8.35 (d, J=8.0 Hz, 1H), 8.13-8.03 (m, 2H), 8.03-8.00 (m, 2H), 7.97 (d, J=8.0 Hz, 1H), 7.78 (br s, 2H), 7.48 (d, J=2.0 Hz, 1H), 7.43 (d, J=8.4 Hz, 2H), 7.36 (d, J=8.4 Hz, 2H), 6.69 (d, J=6.0 Hz, 1H), 4.27-4.14 (m, 1H), 3.94-3.74 (m, 1H), 3.60 (s, 2H), 2.88-2.82 (m, 2H), 2.24-2.12 (m, 2H), 1.97-1.83 (m, 2H), 1.58-1.43 (m, 2H), 1.27-1.23 (m, 2H), 1.16-1.10 (m, 2H).

### Example 632 & 633: 4-((1-(4-(2-(3-Aminopyrazin-2-yl)-5-(2-(cyanomethyl)-2H-1,2,3-triazol-4-yl)-3H-imidazo[4,5-b]pyridin-3-yl)benzyl)piperidin-4-yl)amino)pyrimidine-2-carbonitrile & 2-(4-(2-(3-aminopyrazin-2-yl)-3-(4-((4-((2-cyanopyrimidin-4-yl)amino)piperidin-1-yl)methyl)phenyl)-3H-imidazo[4,5-b]pyridin-5-yl)-2H-1,2,3-triazol-2-yl)acetamide

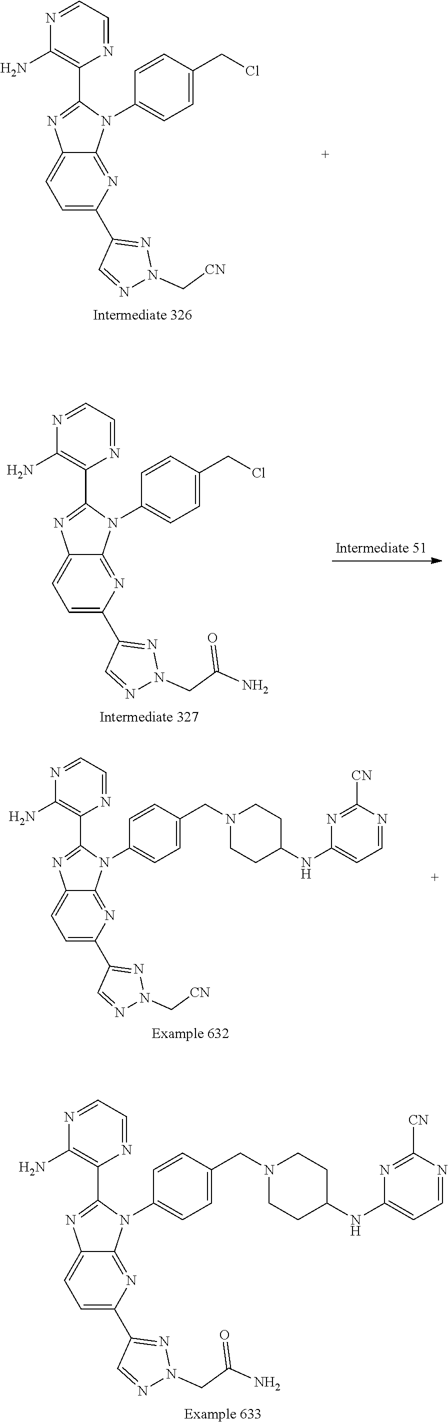 [SMILES: N#CCn1ncc(-c2ccc3nc(-c4nccnc4N)n(-c4ccc(CCl)cc4)c3n2)n1 ; NC(=O)Cn1ncc(-c2ccc3nc(-c4nccnc4N)n(-c4ccc(CCl)cc4)c3n2)n1 ; N#CCn1ncc(-c2ccc3nc(-c4nccnc4N)n(-c4ccc(CN5CCC(Nc6ccnc(C#N)n6)CC5)cc4)c3n2)n1 ; N#Cc1nccc(NC2CCN(Cc3ccc(-n4c(-c5nccnc5N)nc5ccc(-c6cnn(CC(N)=O)n6)nc54)cc3)CC2)n1]

To a solution of a mixture of Intermediate 326 and Intermediate 327 (104 mg, 236 μmol, HCl salt) and Intermediate 51 (57.5 mg, 283 μmol) in DMF (2 mL) were added NaI (3.53 mg, 23.6 μmol) and K₂CO₃ (97.7 mg, 707 μmol). The mixture was stirred at 50° C. for 1 hr. The mixture was diluted with water (10 mL) and extracted with EtOAc (10 mL×2). The combined organic layers were dried over anhydrous Na₂SO₄, filtered and concentrated. After purified by prep-HPLC (column: C18 150×30 mm; mobile phase: [water (NH₃H₂O+NH₄HCO₃)-ACN]; gradient: 47%-77% B over 7 min)), 4-((1-(4-(2-(3-aminopyrazin-2-yl)-5-(2-(cyanomethyl)-2H-1,2,3-triazol-4-yl)-3H-imidazo[4,5-b]pyridin-3-yl)benzyl)piperidin-4-yl)amino)pyrimidine-2-carbonitrile (Example 632, 10.6 mg, yield: 7.7% for three steps) was obtained as a light yellow solid. MS: m/z=610.3 [M+H]⁺. ¹H NMR (400 MHz, Dimethylsulfoxide-d₆) δ 8.39 (d, J=8.4 Hz, 1H), 8.25 (s, 1H), 8.15-8.03 (m, 2H), 8.03-7.97 (m, 2H), 7.78 (br s, 2H), 7.48 (d, J=2.4 Hz, 1H), 7.46-7.41 (m, 2H), 7.39-7.32 (m, 2H), 6.68 (d, J=6.0 Hz, 1H), 5.99 (s, 2H), 3.89-3.71 (m, 1H), 3.59 (s, 2H), 2.92-2.80 (m, 2H), 2.26-2.10 (m, 2H), 1.97-1.83 (m, 2H), 1.62-1.44 (m, 2H). 2-(4-(2-(3-Aminopyrazin-2-yl)-3-(4-((4-((2-cyanopyrimidin-4-yl)amino)piperidin-1-yl)methyl)phenyl)-3H-imidazo[4,5-b]pyridin-5-yl)-2H-1,2,3-triazol-2-yl)acetamide (Example 633, 27.8 mg, yield: 20% for three steps) was also obtained as a light yellow solid. MS: m/z=628.4 [M+H]⁺. ¹H NMR (400 MHz, Dimethylsulfoxide-d₆) δ 8.35 (d, J=8.4 Hz, 1H), 8.18-8.02 (m, 3H), 8.01 (d, J=2.4 Hz, 1H), 7.97 (d, J=8.4 Hz, 1H), 7.77 (br s, 2H), 7.67 (s, 1H), 7.48 (d, J=2.4 Hz, 1H), 7.45-7.41 (m, 2H), 7.40-7.34 (m, 3H), 6.68 (d, J=6.4 Hz, 1H), 5.16 (s, 2H), 3.97-3.69 (m, 1H), 3.59 (s, 2H), 2.88-2.83 (m, 2H), 2.23-2.11 (m, 2H), 1.94-1.82 (m, 2H), 1.58-1.45 (m, 2H)

### Example 634: 4-((1-(4-(2-(3-Aminopyrazin-2-yl)-5-(2-methyl-2H-1,2,3-triazol-4-yl-5-d)-3H-imidazo[4,5-b]pyridin-3-yl)benzyl)piperidin-4-yl)amino)pyrimidine-2-carbonitrile

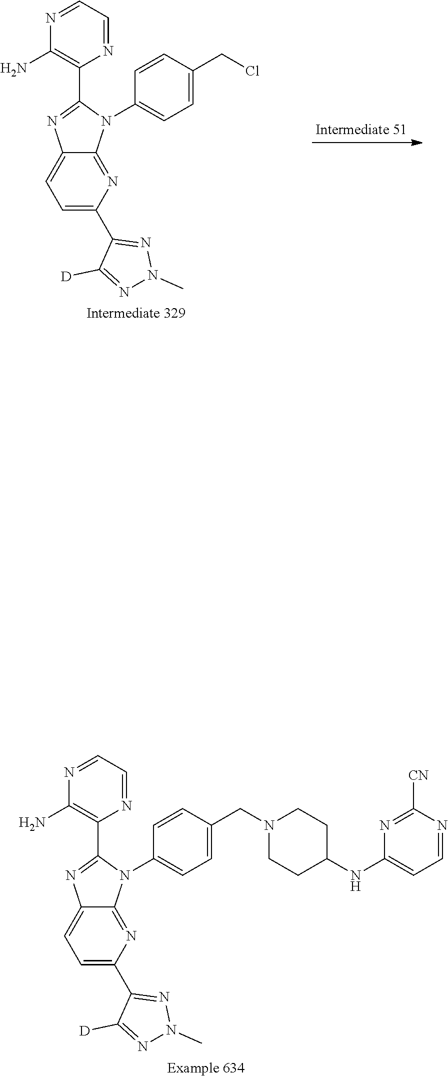 [SMILES: [2H]c1nn(C)nc1-c1ccc2nc(-c3nccnc3N)n(-c3ccc(CCl)cc3)c2n1 ; [2H]c1nn(C)nc1-c1ccc2nc(-c3nccnc3N)n(-c3ccc(CN4CCC(Nc5ccnc(C#N)n5)CC4)cc3)c2n1]

To a solution of Intermediate 329 (375 mg, 824 μmol, HCl salt) and Intermediate 51 (261 mg, 824 μmol, TFA) in DMF (5 mL) were added K₂CO₃ (569 mg, 4.12 mmol) and NaI (12.4 mg, 82.4 μmol). The mixture was stirred at 50° C. for 1 hr. The reaction mixture was quenched with H₂O (5 mL) at 25° C. and extracted with CH₂C2 (15 mL×2). The combined organic layers were washed with brine (50 mL×3), dried over Na₂SO₄, filtered and concentrated under reduced pressure. After purified by prep-HPLC (column: Daisogel SP ODS RPS 150×25 mm×5 μm; mobile phase: [water (NH₄HCO₃)-ACN]; gradient: 42%-72% B over 11 min), 4-((1-(4-(2-(3-aminopyrazin-2-yl)-5-(2-methyl-2H-1,2,3-triazol-4-yl-5-d)-3H-imidazo[4,5-b]pyridin-3-yl)benzyl)piperidin-4-yl)amino)pyrimidine-2-carbonitrile (Example 634, 273 mg, yield: 57% for three steps) was obtained as a yellow lyophilized powder. MS: m/z=586.3 [M+H]⁺. 1D %=98.4%. ¹H NMR (400 MHz, Dimethylsulfoxide-d₆) δ 8.35 (d, J=8.4 Hz, 1H), 8.13-8.05 (m, 2H), 8.02-7.95 (m, 2H), 7.78 (br s, 2H), 7.48 (d, J=2.0 Hz, 1H), 7.45-7.41 (m, 2H), 7.38-7.34 (m, 2H), 6.68 (d, J=6.4 Hz, 1H), 4.21 (s, 3H), 3.89-3.77 (m, 1H), 3.59 (s, 2H), 2.89-2.82 (m, 2H), 2.21-2.12 (m, 2H), 1.96-1.87 (m, 2H), 1.55-1.45 (m, 2H).

### Example 635: 4-((1-(4-(2-(3-Aminopyrazin-2-yl)-5-(2,5-dimethyl-2H-1,2,3-triazol-4-yl)-3H-imidazo[4,5-b]pyridin-3-yl)benzyl)piperidin-4-yl)amino)pyrimidine-2-carbonitrile

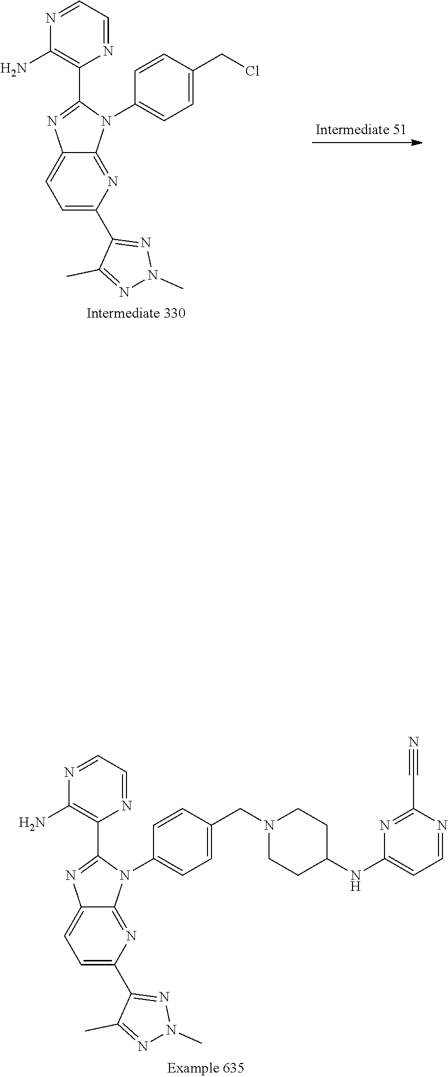 [SMILES: Cc1nn(C)nc1-c1ccc2nc(-c3nccnc3N)n(-c3ccc(CCl)cc3)c2n1 ; Cc1nn(C)nc1-c1ccc2nc(-c3nccnc3N)n(-c3ccc(CN4CCC(Nc5ccnc(C#N)n5)CC4)cc3)c2n1]

To a solution of Intermediate 330 (160 mg, 370 μmol) in DMF (3 mL) were added 4-(piperidin-4-ylamino)pyrimidine-2-carbonitrile (82.8 mg, 408 μmol), K₂CO₃ (154 mg, 1.11 mmol), NaI (5.55 mg, 37.1 μmol). The mixture was stirred at 25° C. for 12 hr under N₂. The mixture was diluted with H₂O (20 mL) and extracted with EtOAc (10 mL×3). The combined organic layers were washed with brine (20 mL), dried over anhydrous Na₂SO₄, filtered and concentrated. After purified by prep-HPLC (column: C18 150×30 mm; mobile phase: [water(NH₃H₂O+NH₄HCO₃)-ACN]; gradient: 47%-77% B over 7 min), 4-((1-(4-(2-(3-aminopyrazin-2-yl)-5-(2,5-dimethyl-2H-1,2,3-triazol-4-yl)-3H-imidazo[4,5-b]pyridin-3-yl)benzyl)piperidin-4-yl)amino)pyrimidine-2-carbonitrile (Example 635, 50.2 mg, yield: 22.7% for three steps) was obtained as a light yellow solid. MS: m/z=599.3 [M+H]⁺. ¹H NMR (400 MHz, Dimethylsulfoxide-d₆) δ 8.30 (br d, J=6.8 Hz, 1H), 8.15-7.98 (m, 3H), 7.98-7.77 (m, 3H), 7.52 (s, 1H), 7.46-7.28 (m, 4H), 6.67 (br s, 1H), 4.11 (s, 3H), 3.87-3.71 (m, 1H), 3.58 (br s, 2H), 2.89-2.73 (m, 2H), 2.27 (s, 3H), 2.17-2.04 (m, 2H), 1.93-1.80 (m, 2H), 1.51-1.48 (m, 2H).

### Example 636: 4-((1-(4-(2-(3-Aminopyrazin-2-yl)-5-(6-methoxypyridin-3-yl)-3H-imidazo[4,5-b]pyridin-3-yl)benzyl)piperidin-4-yl)amino)pyrimidine-2-carbonitrile

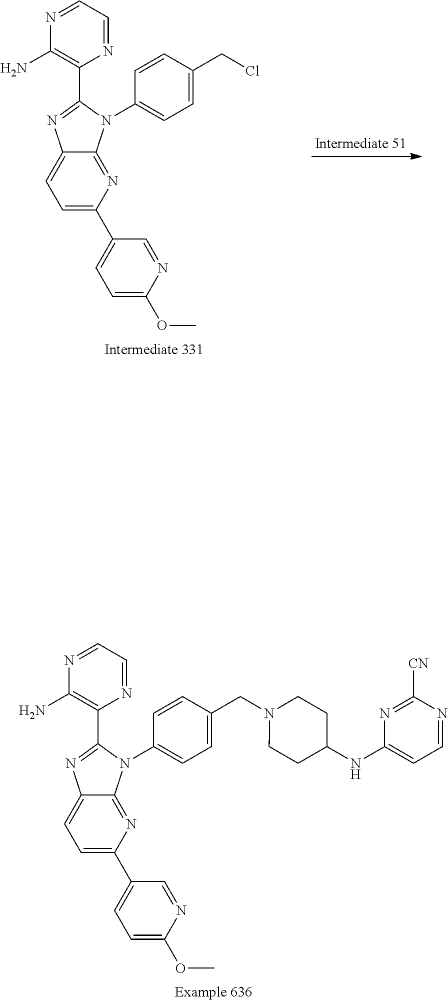 [SMILES: COc1ccc(-c2ccc3nc(-c4nccnc4N)n(-c4ccc(CCl)cc4)c3n2)cn1 ; COc1ccc(-c2ccc3nc(-c4nccnc4N)n(-c4ccc(CN5CCC(Nc6ccnc(C#N)n6)CC5)cc4)c3n2)cn1]

To a solution of Intermediate 331 (197 mg, 410 μmol, HCl salt) and Intermediate 51 (130 mg, 410 μmol, TFA) in DMF (3 mL) were added K₂CO₃ (283 mg, 2.05 mmol) and NaI (12.3 mg, 82.0 μmol). The mixture was stirred at 50° C. for 1 hr. The reaction mixture was quenched with H₂O (5 mL) at 25° C. and extracted with CH₂Cl₂ (15 mL×2). The combined organic layers were washed with brine (50 mL×3), dried over Na₂SO₄, filtered and concentrated under reduced pressure. After purified by prep-HPLC (column: Daisogel SP ODS RPS 150×25 mm×5 μm; mobile phase: [water (NH₄HCO₃)-ACN]; gradient: 38%-68% B over 11 min), 4-((1-(4-(2-(3-aminopyrazin-2-yl)-5-(6-methoxypyridin-3-yl)-3H-imidazo[4,5-b]pyridin-3-yl)benzyl)piperidin-4-yl)amino)pyrimidine-2-carbonitrile (Example 636, 159 mg, yield. 62% for three steps) was obtained as a yellow lyophilized powder. MS: m/z=611.2 [M+H]f. ¹H NMR (400 MHz, Dimethylsulfoxide-d₆) δ 8.84 (d, J=2.4 Hz, 1H), 8.36-8.27 (m, 2H), 8.11-7.98 (m, 4H), 7.82 (br s, 2H), 7.49 (d, J=2.4 Hz, 1H), 7.45-7.36 (m, 4H), 6.91 (d, J=8.8 Hz, 1H), 6.68 (d, J=6.0 Hz, 1H), 3.89 (s, 3H), 3.87-3.77 (m, 1H), 3.60 (s, 2H), 2.91-2.80 (m, 2H), 2.22-2.12 (m, 2H), 1.97-1.83 (m, 2H), 1.60-1.44 (m, 2H).

### Example 637: 4-((1-(4-(2-(3-Aminopyrazin-2-yl)-5-(6-(methoxy-d₃)pyridin-3-yl)-3H-imidazo[4,5-b]pyridin-3-yl)benzyl)piperidin-4-yl)amino)pyrimidine-2-carbonitrile

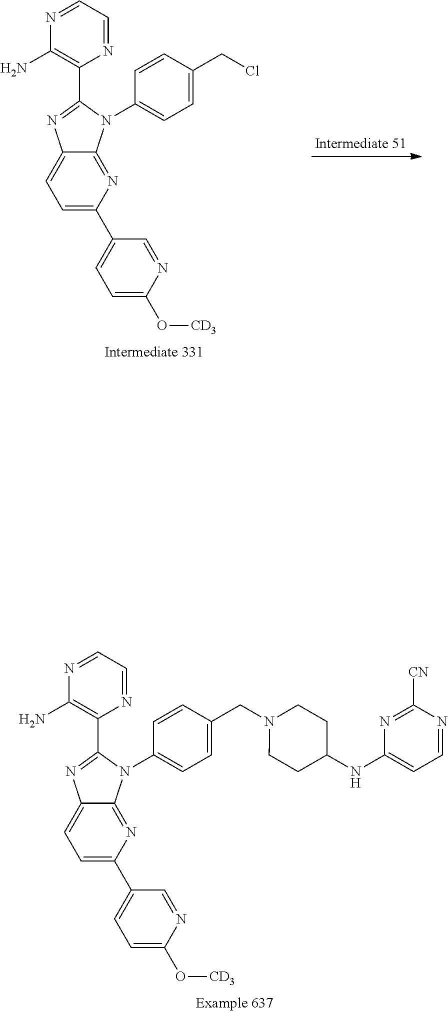 [SMILES: [2H]C([2H])([2H])Oc1ccc(-c2ccc3nc(-c4nccnc4N)n(-c4ccc(CCl)cc4)c3n2)cn1 ; [2H]C([2H])([2H])Oc1ccc(-c2ccc3nc(-c4nccnc4N)n(-c4ccc(CN5CCC(Nc6ccnc(C#N)n6)CC5)cc4)c3n2)cn1]

To a solution of Intermediate 332 (389 mg, 805 μmol, HCl salt) and Intermediate 51 (255 mg, 805 μmol, TFA) in DMF (5 mL) were added K₂CO₃ (556 mg, 4.02 mmol) and NaI (24.1 mg, 161 μmol). The mixture was stirred at 50° C. for 1 hr. The reaction mixture was quenched with H₂O (20 mL) at 25° C. and extracted with CH₂Cl₂ (50 mL×2). The combined organic layers were washed with brine (50 mL×3), dried over Na₂SO₄, filtered and concentrated under reduced pressure. After purified byprep-HPLC (column: Daisogel SP ODS RPS 150×25 mm×5 μm; mobile phase: [water (NH₄HCO₃)-ACN]; gradient: 50%-80% B over 11 min), 4-((1-(4-(2-(3-aminopyrazin-2-yl)-5-(6-(methoxy-d₃)pyridin-3-yl)-3H-imidazo[4,5-b]pyridin-3-yl)benzyl)piperidin-4-yl)amino)pyrimidine-2-carbonitrile (Example 637, 113 mg, yield. 23% for three steps) was obtained as a light yellow lyophilized powder. MS: m/z=614.3 [M+H]⁺. D %: 3D %=100.0%. ¹H NMR (400 MHz, Dimethylsulfoxide-d₆) δ 8.84 (d, J=2.4 Hz, 1H), 8.35-8.26 (m, 2H), 8.10-7.99 (m, 4H), 7.82 (br s, 2H), 7.49 (d, J=2.4 Hz, 1H), 7.44-7.36 (m, 4H), 6.90 (d, J=8.8 Hz, 1H), 6.67 (d, J=6.0 Hz, 1H), 3.88-3.75 (m, 1H), 3.59 (s, 2H), 2.88-2.79 (m, 2H), 2.24-2.13 (m, 2H), 1.97-1.86 (m, 2H), 1.57-1.47 (m, 2H).

### Example 638: 4-((1-(4-(2-(3-Aminopyrazin-2-yl)-5-(5-(methoxy-d₃)pyridin-2-yl)-3H-imidazo[4,5-b]pyridin-3-yl)benzyl)piperidin-4-yl)amino)pyrimidine-2-carbonitrile

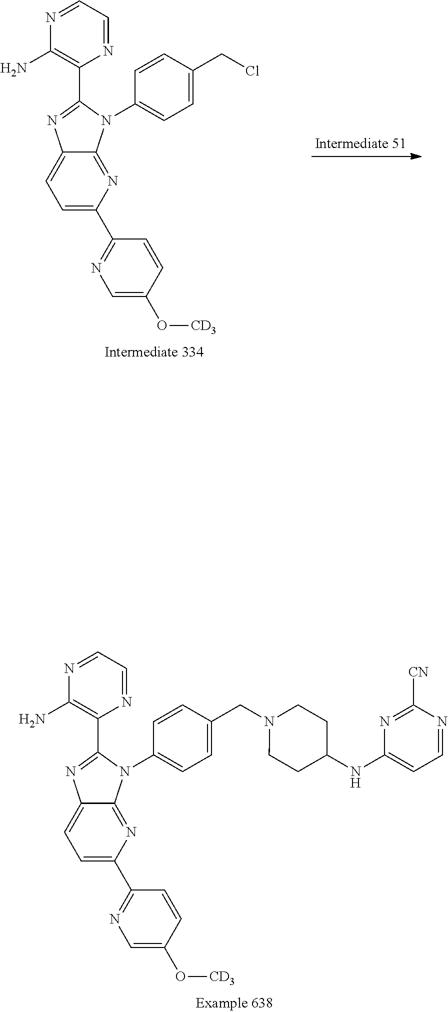 [SMILES: [2H]C([2H])([2H])Oc1ccc(-c2ccc3nc(-c4nccnc4N)n(-c4ccc(CCl)cc4)c3n2)nc1 ; [2H]C([2H])([2H])Oc1ccc(-c2ccc3nc(-c4nccnc4N)n(-c4ccc(CN5CCC(Nc6ccnc(C#N)n6)CC5)cc4)c3n2)nc1]

To a solution of Intermediate 334 (360 mg, 745 μmol, HCl salt) and Intermediate 51 (236 mg, 745 μmol, TFA) in DMF (5 mL) were added K₂CO₃ (515 mg, 3.72 mmol) and NaI (22.3 mg, 149 μmol). The mixture was stirred at 50° C. for 1 hr. The reaction mixture was quenched with H₂O (5 mL) at 25° C. and extracted with CH₂Cl₂ (15 mL×2). The combined organic layers were washed with brine (50 mL×3), dried over Na₂SO₄, filtered and concentrated under reduced pressure. After purified by prep-HPLC (column: Daisogel SP ODS RPS 150×25 mm×5 μm; mobile phase: [water (NH₄HCO₃)-ACN]; gradient: 38%-68% B over 11 min), 4-((1-(4-(2-(3-aminopyrazin-2-yl)-5-(5-(methoxy-d₃)pyridin-2-yl)-3H-imidazo[4,5-b]pyridin-3-yl)benzyl)piperidin-4-yl)amino)pyrimidine-2-carbonitrile (Example 638, 219 mg, yield: 48% for three steps) was obtained as a yellow lyophilized powder. MS: m/z=614.3 [M+H]⁺. D %: 3D %=97.6%. ¹H NMR (400 MHz, Dimethylsulfoxide-d₆) δ 8.43-8.31 (m, 3H), 8.14-8.05 (m, 3H), 8.01 (d, J=2.0 Hz, 1H), 7.81 (br s, 2H), 7.51-7.47 (m, 2H), 7.46-7.42 (m, 2H), 7.41-7.37 (m, 2H), 6.68 (d, J=6.0 Hz, 1H), 3.95-3.77 (m, 1H), 3.61 (s, 2H), 2.91-2.84 (m, 2H), 2.22-2.12 (m, 2H), 1.96-1.86 (m, 2H), 1.57-1.49 (m, 2H).

### Example 639: 4-((1-(4-(2-(3-Aminopyrazin-2-yl)-5-(6-(fluoromethoxy)pyridin-3-yl)-3H-imidazo[4,5-b]pyridin-3-yl)benzyl)piperidin-4-yl)amino)pyrimidine-2-carbonitrile

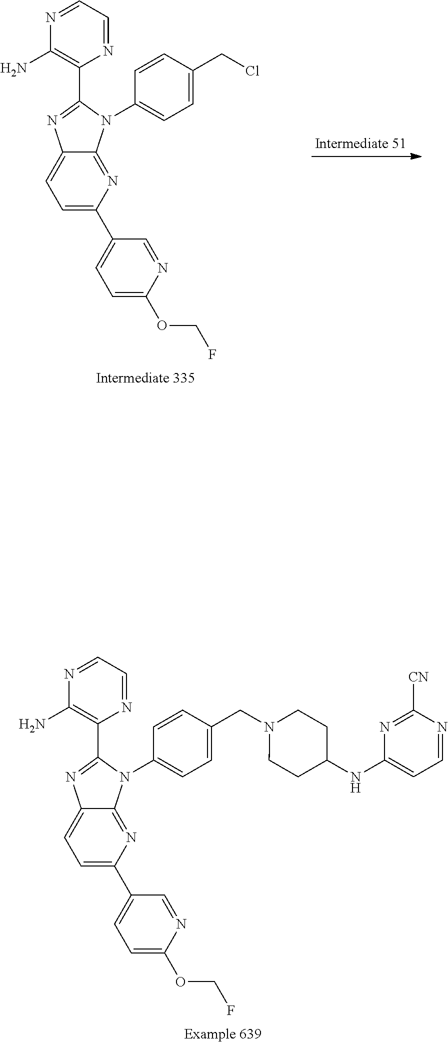 [SMILES: Nc1nccnc1-c1nc2ccc(-c3ccc(OCF)nc3)nc2n1-c1ccc(CCl)cc1 ; N#Cc1nccc(NC2CCN(Cc3ccc(-n4c(-c5nccnc5N)nc5ccc(-c6ccc(OCF)nc6)nc54)cc3)CC2)n1]

To a solution of Intermediate 335 (420 mg, 843 μmol, HCl salt), Intermediate 335 (294 mg, 927 μmol, TFA salt) in DMF (2 mL) were added K₂CO₃ (582 mg, 4.21 mmol) and NaI (37.9 mg, 253 μmol). The mixture was stirred at 25° C. for 16 hr. The reaction mixture was quenched with H₂O (20 mL) and extracted with EtOAc (30 mL×3). The combined organic layers were washed with brine (30 mL×3), dried over anhydrous Na₂SO₄, filtered and concentrated under reduced pressure. After purified by silica gel flash chromatography (Eluent of 0%˜5% MeOH in CH₂Cl₂), 4-((1-(4-(2-(3-aminopyrazin-2-yl)-5-(6-(fluoromethoxy)pyridin-3-yl)-3H-imidazo[4,5-b]pyridin-3-yl)benzyl)piperidin-4-yl)amino)pyrimidine-2-carbonitrile (Example 639, 269 mg, yield: 51% for three steps) was obtained as a yellow solid. MS: m/z=629.2 [M+H]⁺. ¹H NMR (400 MHz, Dimethylsulfoxide-d₆) δ 8.90 (d, J=2.0 Hz, 1H), 8.41 (dd, J=8.4, 2.4 Hz, 1H), 8.37 (d, J=8.4 Hz, 1H), 8.15-8.03 (m, 3H), 8.02 (d, J=2.0 Hz, 1H), 7.82 (br s, 2H), 7.49 (d, J=2.4 Hz, 1H), 7.46-7.41 (m, 2H), 7.41-7.36 (m, 2H), 7.09 (d, J=8.8 Hz, 1H), 6.68 (br d, J=6.4 Hz, 1H), 6.12 (d, J=52.8 Hz, 2H), 3.91-3.76 (m, 1H), 3.60 (s, 2H), 2.89-2.82 (m, 2H), 2.22-2.11 (m, 2H), 1.96-1.84 (m, 2H), 1.57-1.45 (m, 2H). ¹⁹F NMR (400 MHz, Dimethysulfoxide-d₆) δ −154.386.

### Example 640: 4-((1-(4-(2-(3-Aminopyrazin-2-yl)-5-(6-(fluoromethoxy-d₂)pyridin-3-yl)-3H-imidazo[4,5-b]pyridin-3-yl)benzyl)piperidin-4-yl)amino)pyrimidine-2-carbonitrile

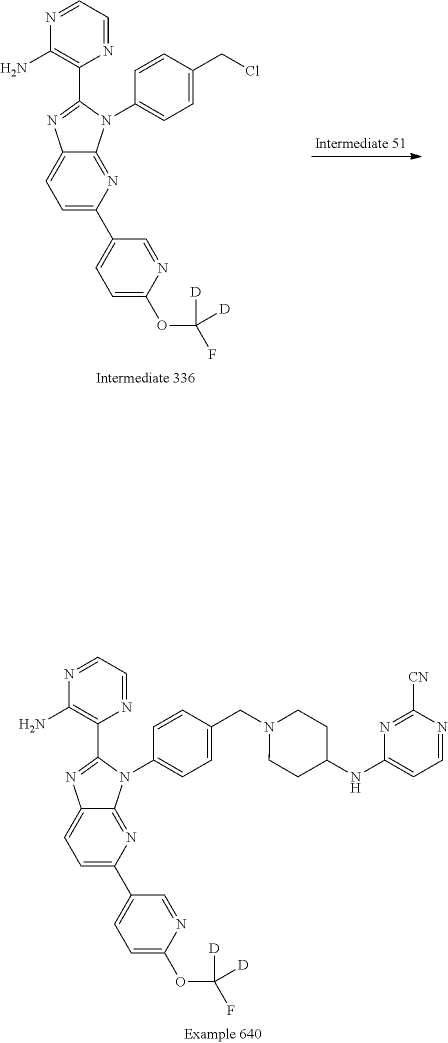 [SMILES: [2H]C([2H])(F)Oc1ccc(-c2ccc3nc(-c4nccnc4N)n(-c4ccc(CCl)cc4)c3n2)cn1 ; [2H]C([2H])(F)Oc1ccc(-c2ccc3nc(-c4nccnc4N)n(-c4ccc(CN5CCC(Nc6ccnc(C#N)n6)CC5)cc4)c3n2)cn1]

To a solution of Intermediate 336 (207 mg, 414 μmol, HCl salt) and Intermediate 51 (131 mg, 414 μmol, TFA) in DMF (5 mL) were added K₂CO₃ (172 mg, 1.24 mmol) and NaI (6.2 mg, 41.4 μmol). The mixture was stirred at 50° C. for 1 hr. The reaction mixture was quenched with H₂O (30 mL) at 25° C. and extracted with CH₂Cl₂ (20 mL×2). The combined organic layers were washed with brine (50 mL×3), dried over Na₂SO₄, filtered and concentrated under reduced pressure. After purified by silica gel flash chromatography (Eluent of 1%˜5% MeOH in CH₂Cl₂) and triturated with EtOAc (10 mL) at 25° C. for 5 min., 4-((1-(4-(2-(3-aminopyrazin-2-yl)-5-(6-(fluoromethoxy-d₂)pyridin-3-yl)-3H-imidazo[4,5-b]pyridin-3-yl)benzyl)piperidin-4-yl)amino)pyrimidine-2-carbonitrile (Example 640, 104 mg, yield: 39% for two steps) was obtained as a yellow solid. MS: m/z=631.2 [M+H]⁺. D %: 2D %=96.6%. ¹H NMR (400 MHz, Dimethylsulfoxide-d₆) δ 8.94-8.86 (m, 1H), 8.44-8.34 (m, 2H), 8.10-8.01 (m, 4H), 7.80 (br s, 2H), 7.51-7.47 (m, 1H), 7.46-7.41 (m, 2H), 7.40-7.36 (m, 2H), 7.09 (d, J=8.4 Hz, 1H), 6.68 (d, J=5.2 Hz, 1H), 3.89-3.77 (m, 1H), 3.60 (s, 2H), 2.88-2.82 (m, 2H), 2.22-2.14 (m, 2H), 1.95-1.87 (m, 2H), 1.56-1.47 (m, 2H). ¹⁹F NMR (400 MHz, Dimethylsulfoxide-d₆) δ −155.59.

### Example 641: 4-((1-(4-(2-(2-Aminopyridin-3-yl)-5-(6-cyclopropoxypyridin-3-yl)-3H-imidazo[4,5-b]pyridin-3-yl)benzyl)piperidin-4-yl)amino)pyrimidine-2-carbonitrile

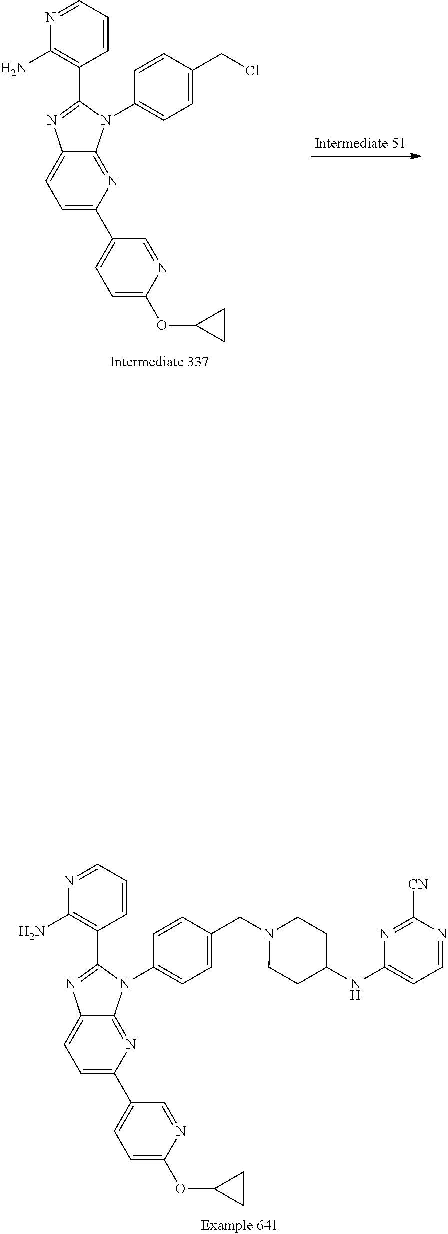 [SMILES: Nc1ncccc1-c1nc2ccc(-c3ccc(OC4CC4)nc3)nc2n1-c1ccc(CCl)cc1 ; N#Cc1nccc(NC2CCN(Cc3ccc(-n4c(-c5cccnc5N)nc5ccc(-c6ccc(OC7CC7)nc6)nc54)cc3)CC2)n1]

To a mixture of Intermediate 337 (191 mg, 407 μmol) and Intermediate 51 (91.1 mg, 448 μmol) in DMF (4 mL) were added NaI (12.2 mg, 81.5 μmol) and K₂CO₃ (169 mg, 1.22 mmol). The mixture was stirred at 80° C. for 12 hr. The mixture was diluted with H₂O (50 mL) and extracted with EtOAc (50 mL×2). The combined organic layers were washed with brine (50 mL), dried over anhydrous Na₂SO₄, filtered and concentrated. After purified by silica gel flash chromatography (Methanol in CH₂Cl₂=0-10%) and prep-HPLC (column: C18 150×30 mm; mobile phase: [water (NH₃H₂O+NH₄HCO₃)-ACN]; gradient: 48%-78% B over 7 min), 4-((1-(4-(2-(2-aminopyridin-3-yl)-5-(6-cyclopropoxypyridin-3-yl)-3H-imidazo[4,5-b]pyridin-3-yl)benzyl)piperidin-4-yl)amino)pyrimidine-2-carbonitrile (Example 641, 50.7 mg, yield 20% for 3 steps) was obtained as a white solid. MS: m/z=658.2 [M+Na]⁺. ¹H NMR (400 MHz, Dimethylsulfoxide-d₆) δ 8.87 (d, J=2.0 Hz, 1H), 8.34-8.24 (m, 2H), 8.12-8.04 (m, 2H), 8.01-7.95 (m, 2H), 7.52-7.43 (m, 4H), 7.16 (d, J=7.2 Hz, 1H), 7.04 (br s, 2H), 6.95 (d, J=8.8 Hz, 1H), 6.67 (d, J=6.4 Hz, 1H), 6.38 (dd, J=7.6, 4.8 Hz, 1H), 4.28-4.19 (m, 1H), 3.90-3.75 (m, 1H), 3.59 (s, 2H), 2.90-2.78 (m, 2H), 2.21-2.11 (m, 2H), 1.94-1.80 (m, 2H), 1.55-1.40 (m, 2H), 0.82-0.73 (m, 2H), 0.70-0.61 (m, 2H).

### Example 642: 4-((1-(4-(2-(2-Aminopyridin-3-yl)-5-(6-isopropoxypyridin-3-yl)-3H-imidazo[4,5-b]pyridin-3-yl)benzyl)piperidin-4-yl)amino)pyrimidine-2-carbonitrile

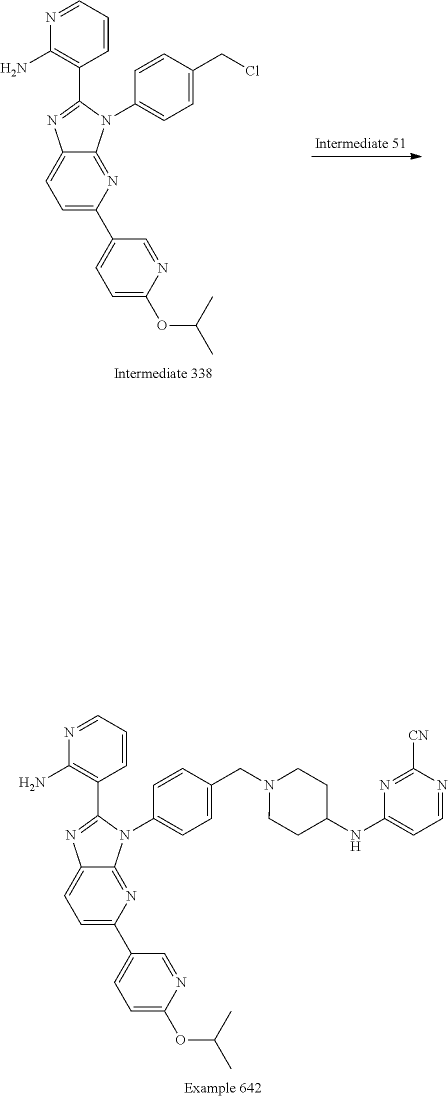 [SMILES: CC(C)Oc1ccc(-c2ccc3nc(-c4cccnc4N)n(-c4ccc(CCl)cc4)c3n2)cn1 ; CC(C)Oc1ccc(-c2ccc3nc(-c4cccnc4N)n(-c4ccc(CN5CCC(Nc6ccnc(C#N)n6)CC5)cc4)c3n2)cn1]

To a mixture of Intermediate 338 (207 mg, 440 μmol) and 4-(piperidin-4-ylamino)pyrimidine-2-carbonitrile (98.3 mg, 483 μmol) in DMF (3 mL) were added NaI (13.2 mg, 87.9 μmol) and K₂CO₃ (182 mg, 1.32 mmol), the mixture was stirred at 20° C. for 12 hr. The reaction mixture was quenched with H₂O (50 mL) and extracted with EtOAc (30 mL×2). The combined organic layers were washed with brine (20 mL), dried over anhydrous Na₂SO₄, filtered and concentrated. After purified by silica gel flash chromatography (MeOH in CH₂Cl₂=0-10%), 4-((1-(4-(2-(2-aminopyridin-3-yl)-5-(6-isopropoxypyridin-3-yl)-3H-imidazo[4,5-b]pyridin-3-yl)benzyl)piperidin-4-yl)amino)pyrimidine-2-carbonitrile (Example 642, 78.7 mg, yield: 28% for three steps) was obtained as a white solid. MS: m/z=660.2 [M+Na]⁺. ¹H NMR (400 MHz, Dimethylsulfoxide-d₆) δ 8.84 (d, J=2.0 Hz, 1H), 8.32-8.23 (m, 2H), 8.12-8.03 (m, 2H), 8.01-7.92 (m, 2H), 7.52-7.41 (m, 4H), 7.15 (dd, J=7.6, 1.6 Hz, 1H), 7.03 (br s, 2H), 6.82 (d, J=8.8 Hz, 1H), 6.67 (d, J=6.0 Hz, 1H), 6.38 (dd, 1=8.0, 4.8 Hz, 1H), 5.32-5.21 (m, 1H), 3.88-3.74 (m, 1H), 3.59 (s, 2H), 2.91-2.76 (m, 2H), 2.21-2.09 (m, 2H), 1.96-1.81 (m, 2H), 1.55-1.43 (m, 2H), 1.33-1.29 (m, 6H).

### Example 643: 4-((1-(4-(2-(2-Aminopyridin-3-yl)-5-(6-isopropylpyridin-3-yl)-3H-imidazo[4,5-b]pyridin-3-yl)benzyl)piperidin-4-yl)amino)pyrimidine-2-carbonitrile

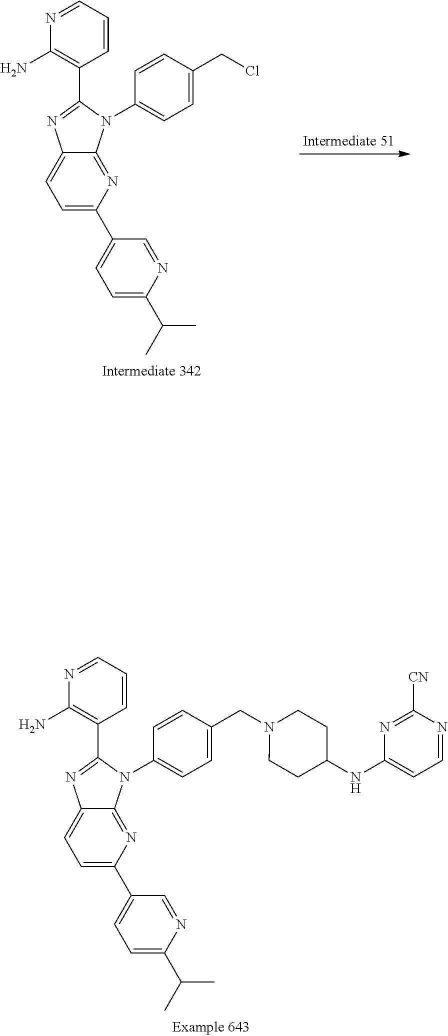 [SMILES: CC(C)c1ccc(-c2ccc3nc(-c4cccnc4N)n(-c4ccc(CCl)cc4)c3n2)cn1 ; CC(C)c1ccc(-c2ccc3nc(-c4cccnc4N)n(-c4ccc(CN5CCC(Nc6ccnc(C#N)n6)CC5)cc4)c3n2)cn1]

To a solution of Intermediate 342 (186 mg, 409 μmol) and Intermediate 51 (99.7 mg, 491 μmol) in DMF (6 mL) were added K₂CO₃ (170 mg, 1.23 mmol) and NaI (12.3 mg, 81.8 μmol) at 20° C. The reaction mixture was stirred at 20° C. for 12 hr and 80° C. for 2 hr. The mixture was diluted with H₂O (20 mL) and extracted with EtOAc (20 mL×2). The combined organic layers were washed with brine (20 mL), dried over anhydrous Na₂SO₄, filtered and concentrated. After purified by prep-HPLC (column: C18 150×30 mm; mobile phase: [water(NH₃H₂O+NH₄HCO₃)-ACN]; gradient: 56%-86% B over 7 min), 4-((1-(4-(2-(2-aminopyridin-3-yl)-5-(6-isopropylpyridin-3-yl)-3H-imidazo[4,5-b]pyridin-3-yl)benzyl)piperidin-4-yl)amino)pyrimidine-2-carbonitrile (Example 643, 64.2 mg, 99.0 μmol, 24% yield for 3 steps) was obtained as a yellow solid. MS: m/z=622.3 [M+H]⁺. ¹H NMR (400 MHz, Dimethylsulfoxide-d₆) δ 9.13-9.11 (m, 1H), 8.32-8.25 (m, 2H), 8.13-7.99 (m, 4H), 7.51-7.44 (m, 4H), 7.36 (d, J=8.0 Hz, 1H), 7.17 (dd, J=8.0, 2.0 Hz, 1H), 7.03 (s, 2H), 6.67 (d, J=6.0 Hz, 1H), 6.39 (dd, J=7.6, 4.8 Hz, 1H), 3.82 (br s, 1H), 3.59 (s, 2H), 3.10-3.00 (m, 1H), 2.88-2.79 (m, 2H), 2.22-2.11 (m, 2H), 1.94-1.84 (m, 2H), 1.55-1.44 (m, 2H), 1.24 (d, J=7.2 Hz, 6H).

### Example 644: 4-((1-(4-(2-(2-Aminopyridin-3-yl)-5-(5-cyclopropylpyridin-2-yl)-3H-imidazo[4,5-b]pyridin-3-yl)benzyl)piperidin-4-yl)amino)pyrimidine-2-carbonitrile

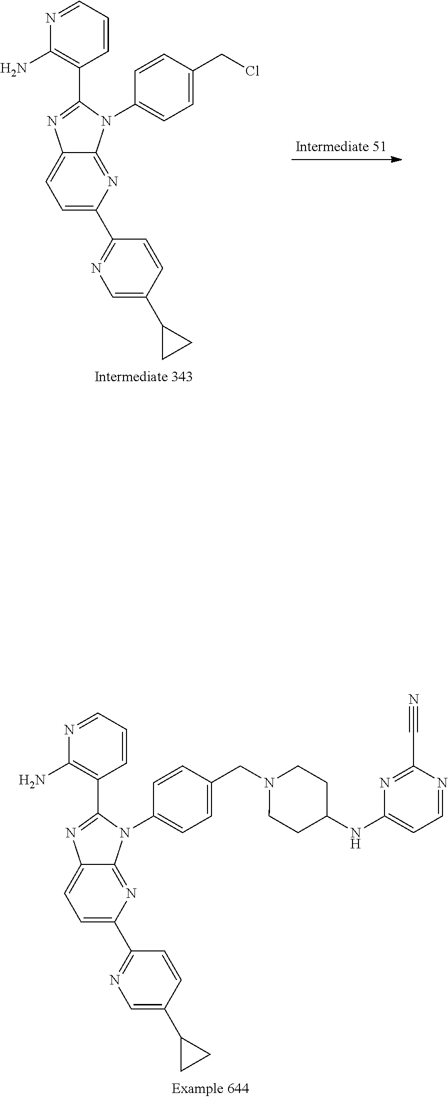 [SMILES: Nc1ncccc1-c1nc2ccc(-c3ccc(C4CC4)cn3)nc2n1-c1ccc(CCl)cc1 ; N#Cc1nccc(NC2CCN(Cc3ccc(-n4c(-c5cccnc5N)nc5ccc(-c6ccc(C7CC7)cn6)nc54)cc3)CC2)n1]

To a solution of Intermediate 343 (238 mg, 525 μmol) in DMF (3 mL) were added Intermediate 51 (128 mg, 631 μmol), K₂CO₃ (218 mg, 1.58 mmol), and NaI (7.88 mg, 52.5 μmol). The mixture was stirred at 25° C. for 12 hr under N₂. The mixture was diluted with H₂O (20 mL) and extracted with EtOAc (10 mL×2). The combined organic layers were washed with brine (15 mL), dried over anhydrous Na₂SO₄, filtered and concentrated. After purified by prep-HPLC (column: Welch Xtimate C18 150×30 mm×5 m; mobile phase: [water(NH₄HCO₃)-ACN]; gradient: 57%-87% B over 7 min), 4-((1-(4-(2-(2-aminopyridin-3-yl)-5-(5-cyclopropylpyridin-2-yl)-3H-imidazo[4,5-b]pyridin-3-yl)benzyl)piperidin-4-yl)amino)pyrimidine-2-carbonitrile (Example 644, 99.6 mg, yield: 30.6% for three steps) was obtained as a light yellow solid. MS: m/z=620.3 [M+H]⁺. ¹H NMR (400 MHz, Dimethylsulfoxide-d₆) δ 8.49 (d, J=2.0 Hz, 1H), 8.42 (d, J=8.4 Hz, 1H), 8.28 (d, J=8.4 Hz, 1H), 8.13-8.03 (m, 3H), 8.00 (dd, J=4.8, 2 Hz, 1H), 7.53-7.43 (m, 5H), 7.16 (dd, J=7.6, 2.4 Hz, 1H), 7.02 (br s, 2H), 6.68 (d, J=6.0 Hz, 1H), 6.38 (dd, J=7.6, 4.8 Hz, 1H), 3.89-3.74 (m, 1H), 3.60 (s, 2H), 2.90-2.76 (m, 2H), 2.23-2.16 (m, 2H), 2.15-1.95 (m, 1H), 1.94-1.84 (m, 2H), 1.59-1.47 (m, 2H), 1.07-1.03 (m, 2H), 0.82-0.77 (m, 2H).

### Example 645: 4-((1-(4-(2-(2-Aminopyridin-3-yl)-5-(6-(difluoromethyl)pyridin-3-yl)-3H-imidazo[4,5-b]pyridin-3-yl)benzyl)piperidin-4-yl)amino)pyrimidine-2-carbonitrile

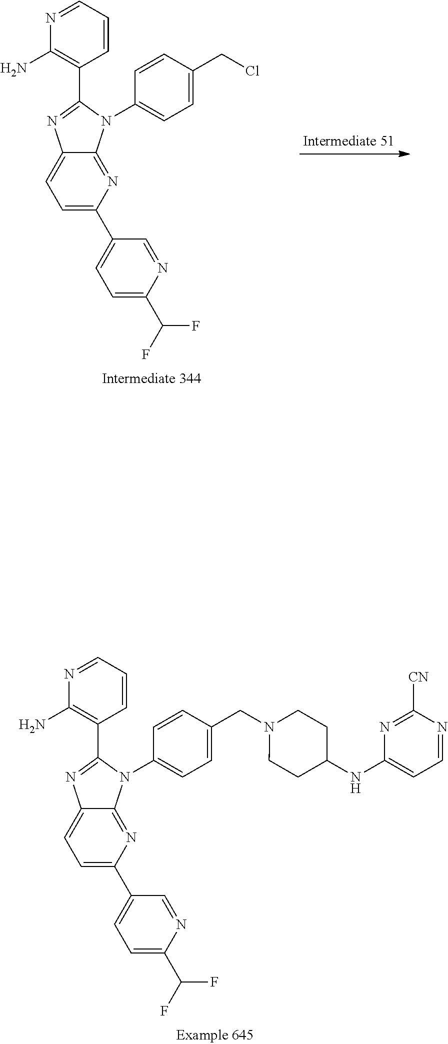 [SMILES: Nc1ncccc1-c1nc2ccc(-c3ccc(C(F)F)nc3)nc2n1-c1ccc(CCl)cc1 ; N#Cc1nccc(NC2CCN(Cc3ccc(-n4c(-c5cccnc5N)nc5ccc(-c6ccc(C(F)F)nc6)nc54)cc3)CC2)n1]

To a solution of Intermediate 344 (405 mg, 811 μmol, HCl salt) and Intermediate 51 (260 mg, 820 μmol, TFA salt) in DMF (4 mL) were added K₂CO₃ (336 mg, 2.43 mmol) and NaI (12.2 mg, 81.1 μmol). The mixture was stirred at 25° C. for 16 hr. The reaction mixture was quenched with H₂O (20 mL) and extracted with EtOAc (20 mL×3). The combined organic layers were washed with brine (20 mL×2), dried over Na₂SO₄, filtered and concentrated under reduced pressure. After purified by silica gel flash chromatography (Eluent of 0%˜6% MeOH in CH₂Cl₂), 4-((1-(4-(2-(2-aminopyridin-3-yl)-5-(6-(difluoromethyl)pyridin-3-yl)-3H-imidazo[4,5-b]pyridin-3-yl)benzyl)piperidin-4-yl)amino)pyrimidine-2-carbonitrile (Example 645, 227.8 mg, yield: 45% for three steps) was obtained as a brown solid. MS: m/z=630.2 [M+H]. ¹H NMR (400 MHz, Dimethylsulfoxide-d₆) δ 9.32 (s, 1H), 8.64-8.48 (m, 1H), 8.36 (d, J=8.4 Hz, 1H), 8.15 (d, J=8.4 Hz, 1H), 8.11-8.03 (m, 2H), 8.03-7.99 (m, 1H), 7.80 (d, J=8.0 Hz, 1H), 7.52-7.43 (m, 4H), 7.20-7.13 (m, 1H), 7.14-6.86 (m, 3H), 6.67 (d, J=5.6 Hz, 1H), 6.39 (dd, J=7.6, 4.8 Hz, 1H), 3.89-3.74 (m, 1H), 3.59 (s, 2H), 2.90-2.75 (m, 2H), 2.21-2.10 (m, 2H), 1.98-1.77 (m, 2H), 1.60-1.40 (m, 2H). ¹⁹F NMR (400 MHz, Dimethysulfoxide-<d₆) δ −115.381.

### Example 646: 4-((1-(4-(2-(2-Aminopyridin-3-yl)-5-(6-(trifluoromethyl)pyridin-3-yl)-3H-imidazo[4,5-b]pyridin-3-yl)benzyl)piperidin-4-yl)amino)pyrimidine-2-carbonitrile

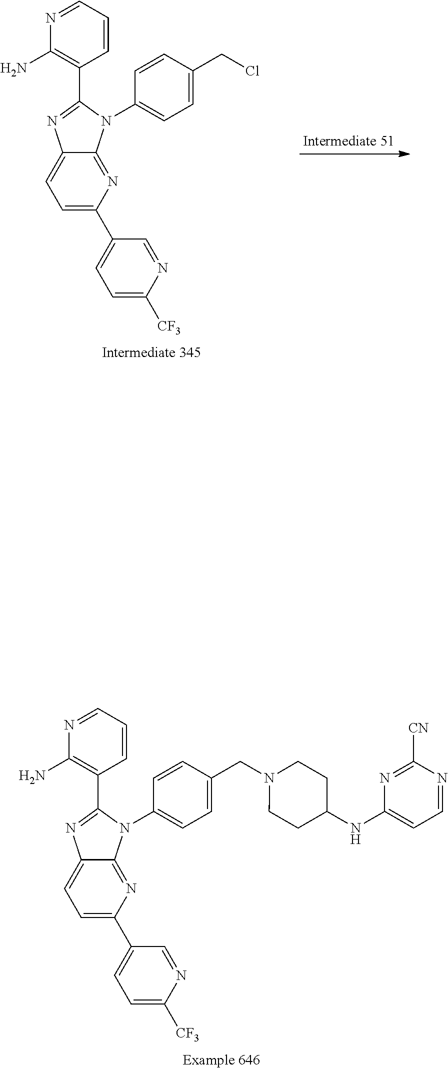 [SMILES: Nc1ncccc1-c1nc2ccc(-c3ccc(C(F)(F)F)nc3)nc2n1-c1ccc(CCl)cc1 ; N#Cc1nccc(NC2CCN(Cc3ccc(-n4c(-c5cccnc5N)nc5ccc(-c6ccc(C(F)(F)F)nc6)nc54)cc3)CC2)n1]

To a solution of Intermediate 345 (276 mg, 574 μmol, HCl salt) and Intermediate 51 (200 mg, 631 μmol, TFA salt) in DMF (2 mL) were added K₂CO₃ (238 mg, 1.72 mmol) and NaI (8.60 mg, 57.4 μmol). The mixture was stirred at 25° C. for 16 hr. The reaction mixture was quenched with H₂O (20 mL) and extracted with EtOAc (20 mL×3). The combined organic layers were washed with brine (20 mL×2), dried over Na₂SO₄, filtered and concentrated under reduced pressure. After purified by silica gel flash chromatography (Eluent of 0%˜3% MeOH in CH₂Cl₂), 4-((1-(4-(2-(2-aminopyridin-3-yl)-5-(6-(trifluoromethyl)pyridin-3-yl)-3H-imidazo[4,5-b]pyridin-3-yl)benzyl)piperidin-4-yl)amino)pyrimidine-2-carbonitrile (Example 646, 199.8 mg, yield: 54% for three steps) was obtained as a light yellow solid. MS: m/z=648.2 [M+H]⁺. ¹H NMR (400 MHz, Methanol-d₆) 9.45 (s, 1H), 8.72-8.48 (m, 1H), 8.29 (d, J=8.8 Hz, 1H), 8.10 (d, J=8.4 Hz, 1H), 8.04-7.94 (m, 2H), 7.88 (d, J=8.0 Hz, 1H), 7.57 (d, J=8.4 Hz, 2H), 7.47 (d, J=8.4 Hz, 2H), 7.38-7.28 (m, 1H), 6.67-6.52 (m, 1H), 6.48 (dd, J=7.6, 4.8 Hz, 1H), 4.07-3.88 (m, 1H), 3.67 (s, 2H), 3.05-2.89 (m, 2H), 2.36-2.20 (m, 2H), 2.05-1.98 (m, 2H), 1.59-1.58 (m, 2H). ¹⁹F NMR (400 MHz, Dimethysulfoxide-d₆) δ −69.247.

### Example 647: 4-((1-(4-(2-(2-Aminopyridin-3-yl)-5-(5-ethylpyridin-2-yl)-3H-imidazo[4,5-b]pyridin-3-yl)benzyl)piperidin-4-yl)amino)pyrimidine-2-carbonitrile

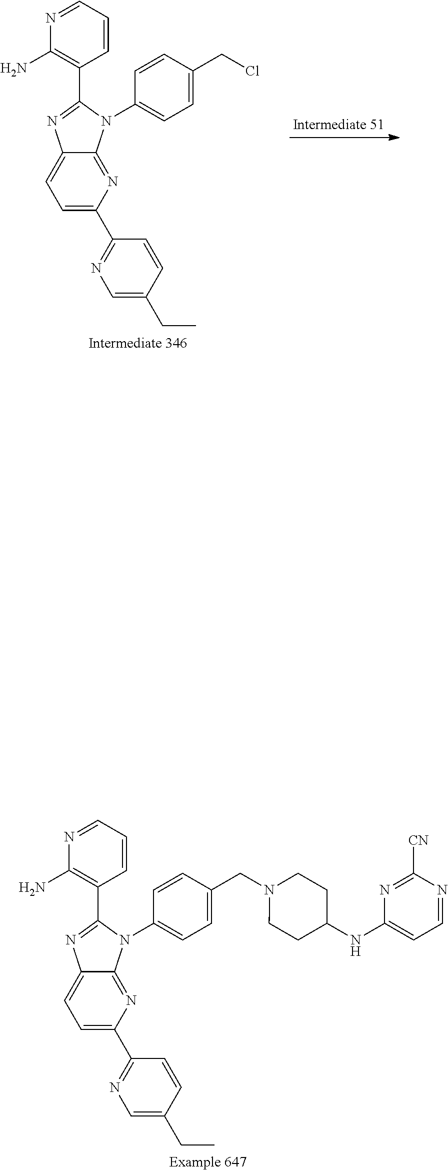 [SMILES: CCc1ccc(-c2ccc3nc(-c4cccnc4N)n(-c4ccc(CCl)cc4)c3n2)nc1 ; CCc1ccc(-c2ccc3nc(-c4cccnc4N)n(-c4ccc(CN5CCC(Nc6ccnc(C#N)n6)CC5)cc4)c3n2)nc1]

To a solution of Intermediate 346 (237 mg, 496 μmol, HCl salt) and Intermediate 51 (158 mg, 496 μmol, TFA salt) in DMF (5 mL) were added K₂CO₃ (343 mg, 2.48 mmol) and NaI (14.9 mg, 99.3 μmol). The mixture was stirred at 50° C. for 1 hr. The reaction mixture was quenched with H₂O (10 mL) at 25° C. and extracted with CH₂Cl₂ (15 mL×2). The combined organic layers were washed with brine (50 mL×3), dried over Na₂SO₄, filtered and concentrated under reduced pressure. After purified by prep-HPLC (column: CD07-Daisogel SP-100-8-ODS-PK 150×25×10 μm; mobile phase: [water (NH₄HCO₃)-ACN]; gradient: 42%-72% B over 10 min), 4-((1-(4-(2-(2-aminopyridin-3-yl)-5-(5-ethylpyridin-2-yl)-3H-imidazo[4,5-b]pyridin-3-yl)benzyl)piperidin-4-yl)amino)pyrimidine-2-carbonitrile (Example 647, 100 mg, yield: 33% for two steps) was obtained as a yellow lyophilized powder. MS: m/z=608.3 [M+H]⁺. ¹H NMR (400 MHz, Dimethylsulfoxide-d₆) δ 8.53 (d, J=1.6 Hz, 1H), 8.43 (d, J=8.4 Hz, 1H), 8.29 (d, J=8.4 Hz, 1H), 8.14-8.02 (m, 3H), 8.00 (dd, J=4.8, 1.6 Hz, 1H), 7.74 (dd, J=8.0, 1.2 Hz, 1H), 7.53-7.43 (m, 4H), 7.19-7.13 (m, 1H), 7.02 (br s, 2H), 6.68 (d, J=6.0 Hz, 1H), 6.38 (dd, J=7.6, 4.8 Hz, 1H), 3.91-3.74 (m, 1H), 3.60 (s, 2H), 2.89-2.79 (m, 2H), 2.66 (q, J=7.6 Hz, 2H), 2.22-2.12 (m, 2H), 1.94-1.81 (m, 2H), 1.57-1.45 (m, 2H), 1.21 (t, J=7.6 Hz, 3H).

### Example 648: 4-((1-(4-(2-(2-Aminopyridin-3-yl)-5-(5-(fluoromethyl)pyridin-2-yl)-3H-imidazo[4,5-b]pyridin-3-yl)benzyl)piperidin-4-yl)amino)pyrimidine-2-carbonitrile

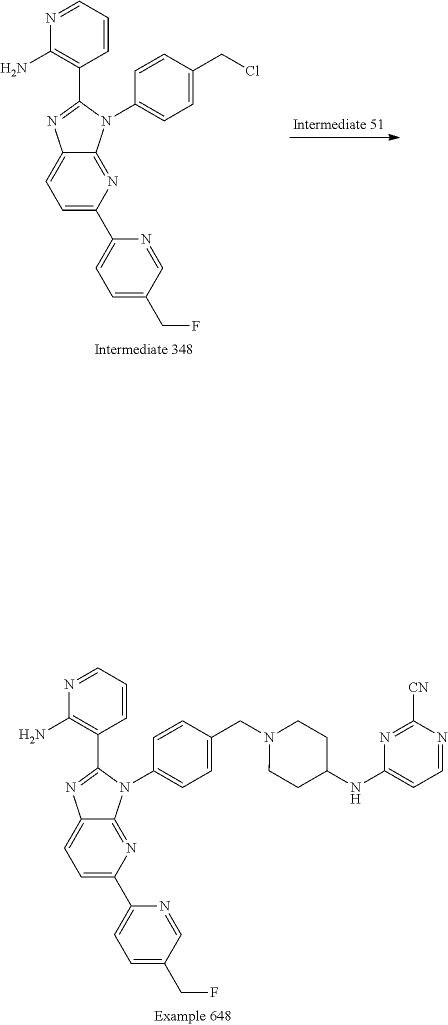 [SMILES: Nc1ncccc1-c1nc2ccc(-c3ccc(CF)cn3)nc2n1-c1ccc(CCl)cc1 ; N#Cc1nccc(NC2CCN(Cc3ccc(-n4c(-c5cccnc5N)nc5ccc(-c6ccc(CF)cn6)nc54)cc3)CC2)n1]

To a solution of Intermediate 348 (158 mg, 328 μmol, HCl salt) and Intermediate 51 (104 mg, 328 μmol, TFA) in DMF (5 mL) were added K₂CO₃ (227 mg, 1.64 mmol) and NaI (9.84 mg, 65.7 μmol). The mixture was stirred at 50° C. for 1 hr. The reaction mixture was quenched with H₂O (5 mL) at 25° C. and extracted with CH₂Cl₂ (15 mL×2). The combined organic layers were washed with brine (50 mL×3), dried over Na₂SO₄, filtered and concentrated under reduced pressure. After purified by silica gel flash chromatography (Eluent of 0%˜6% MeOH in CH₂Cl₂), 4-((1-(4-(2-(2-aminopyridin-3-yl)-5-(5-(fluoromethyl)pyridin-2-yl)-3H-imidazo[4,5-b]pyridin-3-yl)benzyl)piperidin-4-yl)amino)pyrimidine-2-carbonitrile (Example 648, 116 mg, yield: 52% for three steps) was obtained as a yellow solid. MS: m/z=612.3 [M+H]⁺. ¹H NMR (400 MHz, Dimethylsulfoxide-d₆) δ 8.73 (s, 1H), 8.49 (d, J=8.4 Hz, 1H), 8.32 (d, J=8.4 Hz, 1H), 8.23 (d, J=8.4 Hz, 1H), 8.13-8.02 (m, 2H), 8.01 (dd, J=4.8, 2.0 Hz, 1H), 7.99-7.94 (m, 1H), 7.52-7.46 (m, 4H), 7.17 (dd, J=7.6, 1.6 Hz, 1H), 7.01 (br s, 2H), 6.68 (d, J=6.0 Hz, 1H), 6.39 (dd, J=7.6, 4.8 Hz, 1H), 5.53 (d, J=47.2 Hz, 2H), 3.93-3.71 (m, 1H), 3.61 (s, 2H), 2.89-2.80 (m, 2H), 2.23-2.13 (m, 2H), 1.95-1.85 (m, 2H), 1.58-1.46 (m, 2H). ¹⁹F NMR (400 MHz, Dimethylsulfoxide-d₆) δ −206.809.

### Example 649: 4-((1-(4-(2-(2-Aminopyridin-3-yl)-5-(5-(difluoromethyl)pyridin-2-yl)-3H-imidazo[4,5-b]pyridin-3-yl)benzyl)piperidin-4-yl)amino)pyrimidine-2-carbonitrile

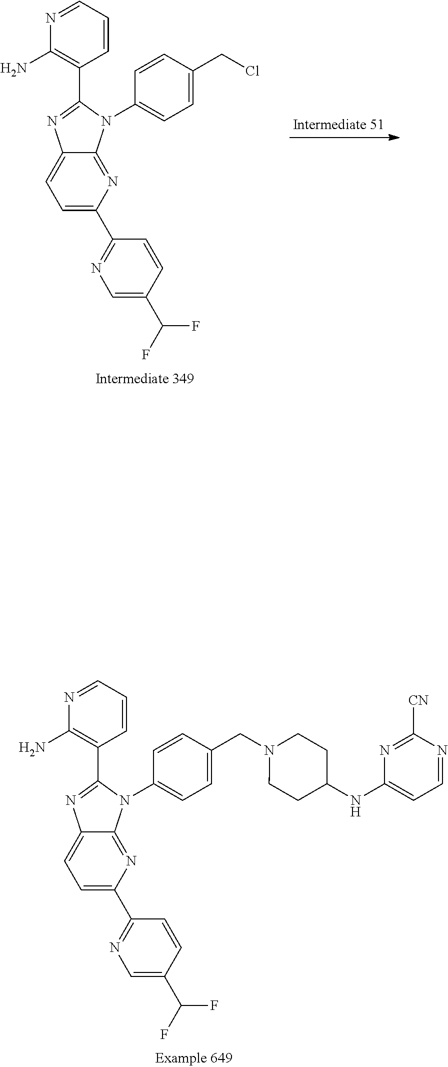 [SMILES: Nc1ncccc1-c1nc2ccc(-c3ccc(C(F)F)cn3)nc2n1-c1ccc(CCl)cc1 ; N#Cc1nccc(NC2CCN(Cc3ccc(-n4c(-c5cccnc5N)nc5ccc(-c6ccc(C(F)F)cn6)nc54)cc3)CC2)n1]

To a solution of Intermediate 349 (270 mg, 541 μmol, HCl salt) and Intermediate 51 (172 mg, 541 μmol, TFA salt) in DMF (5 mL) were added K₂CO₃ (299 mg, 1.16 mmol) and NaI (41 mg, 270 μmol). The mixture was stirred at 25° C. for 16 hr. The reaction mixture was with H₂O (5 mL) at 25° C. and extracted with EtOAc (10 mL×3). The combined organic layers were washed with brine (10 mL×3), dried over Na₂SO₄, filtered and concentrated under reduced pressure. After purified by prep-HPLC (column: CD07-Daisogel SP-100-8-ODS-PK 150×25×10 μm; mobile phase. [water (NH₄HCO₃)-ACN]; gradient: 38%-68% B over 10 min), 4-((1-(4-(2-(2-aminopyridin-3-yl)-5-(5-(difluoromethyl)pyridin-2-yl)-3H-imidazo[4,5-b]pyridin-3-yl)benzyl)piperidin-4-yl)amino)pyrimidine-2-carbonitrile (Example 649, 170.4 mg, yield: 48% for two steps) was obtained as a yellow powder. MS: m/z=630.3 [M+H]⁺. ¹H NMR (400 MHz, Dimethylsulfoxide-d₆) δ 8.87 (s, 1H), 8.51 (d, J=8.4 Hz, 1H), 8.37-8.29 (m, 2H), 8.14-8.00 (m, 4H), 7.53-7.46 (m, 4H), 7.35-7.16 (m, 2H), 7.06-7.00 (m, 2H), 6.68 (d, J=6.0 Hz, 1H), 6.39 (dd, J=7.6, 4.8 Hz, 1H), 3.90-3.72 (m, 1H), 3.60 (s, 2H), 2.88-2.80 (m, 2H), 2.22-2.12 (m, 2H), 1.97-1.86 (m, 2H), 1.58-1.47 (m, 2H). ¹⁹F NMR (400 MHz, Dimethylsulfoxide-d₆) δ −111.254.

### Example 650: 4-((1-(4-(2-(2-Aminopyridin-3-yl)-5-(5-(trifluoromethyl)pyridin-2-yl)-3H-imidazo[4,5-b]pyridin-3-yl)benzyl)piperidin-4-yl)amino)pyrimidine-2-carbonitrile

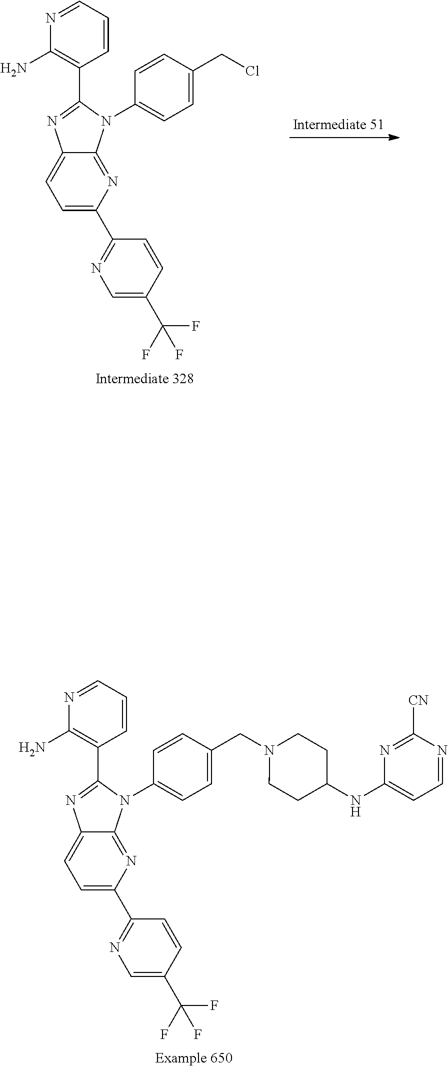 [SMILES: Nc1ncccc1-c1nc2ccc(-c3ccc(C(F)(F)F)cn3)nc2n1-c1ccc(CCl)cc1 ; N#Cc1nccc(NC2CCN(Cc3ccc(-n4c(-c5cccnc5N)nc5ccc(-c6ccc(C(F)(F)F)cn6)nc54)cc3)CC2)n1]

To a solution of Intermediate 328 (246 mg, 476 μmol, HCl salt) and Intermediate 51 (151 mg, 476 μmol, TFA salt) in DMF (5 mL) were added K₂CO₃ (263 mg, 1.9 mmol) and NaI (36 mg, 238 μmol). The mixture was stirred at 25° C. for 16 hr. The reaction mixture was quenched with H₂O (10 mL) at 25° C. and extracted with EtOAc (10 mL×3). The combined organic layers were washed with brine (10 mL×3), dried over Na₂SO₄, filtered and concentrated under reduced pressure. After purified by silica gel flash chromatography (Eluent of 0%˜5% MeOH in CH₂Cl₂), 4-((1-(4-(2-(2-aminopyridin-3-yl)-5-(5-(trifluoromethyl)pyridin-2-yl)-3H-imidazo[4,5-b]pyridin-3-yl)benzyl)piperidin-4-yl)amino)pyrimidine-2-carbonitrile (Example 650, 166.4 mg, yield: 53% for two steps) was obtained as a light yellow powder. MS: m/z=648.6 [M+H]⁺. ¹H NMR (400 MHz, Dimethylsulfoxide-d₆) δ 9.06 (s, 1H), 8.53 (d, J=8.4 Hz, 1H), 8.41-8.26 (m, 3H), 8.21-7.97 (m, 3H), 7.57-7.42 (m, 4H), 7.18 (d, J=7.6 Hz, 1H), 7.03 (br s, 2H), 6.68 (d, J=5.6 Hz, 1H), 6.43-6.35 (m, 1H), 3.92-3.72 (m, 1H), 3.60 (s, 2H), 2.90-2.78 (m, 2H), 2.22-2.10 (m, 2H), 1.96-1.83 (m, 2H), 1.57-1.44 (m, 2H). ¹⁹F NMR (400 MHz, Dimethylsulfoxide-d₆) δ −60.763.

### II. Biological Evaluation

### Example 1: NanoBRET Target Engagement (TE) Assay

NanoBRET is a highly specific and validated cell-based technique for assessing target engagement (Vasta et al., 2018, Cell Chem Biol. 25(2):206-214). The NanoBRET™ Target Engagement (TE) Intracellular Kinase Assays are based on the NanoBRET™ System (Promega Corporation), an energy transfer technique designed to measure molecular proximity in living cells. The NanoBRET™ TE Assays measure the apparent affinity of test compounds by competitive displacement of the NanoBRET™ tracer compound, which is a cell permeable molecule engineered to be reversibly bound to a NanoLuc® luciferase-kinase fusion expressed in cells. For compound screening, when a test compound binds to the selected kinase, the BRET signal is attenuated. For kinase inhibitors in particular, intracellular target selectivity is fundamental to pharmacological mechanism and allows the proteins of interest to be in the correct cellular confirmation. Although non-cell-based techniques have been developed to measure kinase binding or enzymatic inhibition with accuracy and precision, such approaches can fail to accurately predict engagement of the full-length target protein in the more complex and biologically relevant cellular context (Knight and Shokat, 2005, Chem. Biol. 12, 621-637; Smyth and Collins, 2009, J. Chem. Biol. 2, 131-151). The NanoBRET assay procedure was used to interrogate the compounds against the full length AKT E17K per manufacturers suggestions. Briefly, HEK-293 cells (ATCC Cat #CRL-1573) were used for transfection purposes using FuGENE HD Transfection Reagent (Promega Cat #E2311). All cells were evaluated for viability prior to transfection and optimization of the transfection was done prior to experimentation. Greater than 95% viability was used for all experiments. Following transfection, cells were washed and resuspended in Opti-MEM. NanoBRET assays were performed in white, 384-well plates (Corning) at a density of 2×10⁵ cells/well. All example compounds were prepared as concentrated stock solutions in DMSO (Sigma-Aldrich). Compounds are dissolved in DMSO to make 10 mM stock solution. Example compounds were transferred as 40 uL of 10 mM stock solution to a 384 pp-plate (LABCYTE, PP-0200) and diluted in 3-fold, 10-point dilution via transferring 12 μL compound into 24 μL DMSO by Apricot liquid handler. A Labcyte ECHO 550 compound dispenser was used to facilitate compound transfer directly to cells. Cells were equilibrated for 2 hr with energy transfer probes and example compound prior to BRET measurements. The AKTE17K (Promega Cat #NV2421) as well as specific probe (NanoBRET tracer, Promega Cat #N264B) was prepared at a concentration of 20× in tracer dilution buffer (12.5 mM HEPES, 31.25% PEG-400, pH 7.5). For target engagement analysis, the energy transfer probes were added to the cells at concentrations optimized for the target in question (AKT E17K). Following compound incubation, NanoBRET NanoGlo Substrate (Promega Cat #N157D) and Extracellular Nanoluc Inhibitor (Promega Cat #N235C) was added according to the manufacturer's recommended protocol, and luminescence was measured on Envision Reader (Perkin Elmer) Multimode Luminometer equipped with 450 nm BPfilter (donor) and 600 nm LPfilter (acceptor), using 0.5 s integration time. Milli-BRET units (mBU) are calculated by multiplying the raw BRET values by 1000. Apparent tracer affinity values (EC50) were determined using the sigmoidal dose-response (variable slope). Competitive displacement data were then plotted and data were fit to determine the EC₅₀ value for each example compound. Table 4 provides the assay results for select examples. Activity is defined as “+”, for EC₅₀ between 600-10,000 nanomolar; “++” for EC₅₀ between 60-600 nanomolar; “+++” for EC₅₀ between 15-60 nanomolar; and “++++” for EC₅₀ less than 15 nanomolar.

TABLE 4

| | | | AKT1 |
|---|---|---|---|
| Chemistry | | MS: | E17K |
| Example | Compound Name | [M + H]⁺ | IC50 |
| 1 | 6-((1-(4-(2-(2-aminopyridin-3-yl)-3H-imidazo[4,5-b]pyridin-3- | 503.1 | + |
| | yl)benzyl)piperidin-4-yl)oxy)nicotinonitrile | | |
| 2 | 6-((1-(4-(2-(2-aminopyridin-3-yl)-5-phenyl-3H-imidazo[4,5- | 579.3 | ++ |
| | b]pyridin-3-yl)benzyl)piperidin-4-yl)oxy)nicotinonitrile | | |
| 3 | 6-((1-(4-(2-(2-aminopyridin-3-yl)-6-phenyl-3H-imidazo[4,5- | 579.1 | + |
| | bjpyridin-3-yl)benzyl)piperidin-4-yl)oxy)nicotinonitrile | | |
| 4 | 6-((1-(4-(2-(2-aminopyridin-3-yl)-3H-imidazo[4,5-b]pyridin-3- | 503.3 | + |
| | yl)benzyl)piperidin-4-yl)oxy)picolinonitrile | | |
| 5 | 2-((1-(4-(2-(2-aminopyridin-3-yl)-3H-imidazo[4,5-b]pyridin-3- | 503.3 | + |
| | yl)benzyl)piperidin-4-yl)oxy)isonicotinonitrile | | |
| 6 | 2-((1-(4-(2-(2-aminopyridin-3-yl)-3H-imidazo[4,5-b]pyridin-3- | 503.4 | + |
| | yl)benzyl)piperidin-4-yl)oxy)nicotinonitrile | | |
| 7 | 6-((1-(4-(2-(2-aminopyridin-3-yl)-5-phenyl-3H-imidazo[4,5- | 579.3 | ++ |
| | b]pyridin-3-yl)benzyl)piperidin-4-yl)oxy)picolinonitrile | | |
| 8 | 2-((1-(4-(2-(2-aminopyridin-3-yl)-5-phenyl-3H-imidazo[4,5- | 579.1 | + |
| | b]pyridin-3-yl)benzyl)piperidin-4-yl)oxy)isonicotinonitrile | | |
| 9 | 2-((1-(4-(2-(2-aminopyridin-3-yl)-5-phenyl-3H-imidazo[4,5- | 579.1 | ++ |
| | b]pyridin-3-yl)benzyl)piperidin-4-yl)oxy)nicotinonitrile | | |
| 10 | 6-((1-(4-(2-(2-aminopyridin-3-yl)-6-phenyl-3H-imidazo[4,5- | 579.3 | + |
| | b]pyridin-3-yl)benzyl)piperidin-4-yl)oxy)picolinonitrile | | |
| 11 | 2-((1-(4-(2-(2-aminopyridin-3-yl)-6-phenyl-3H-imidazo[4,5- | 579.3 | + |
| | b]pyridin-3-yl)benzyl)piperidin-4-yl)oxy)isonicotinonitrile | | |
| 12 | 2-((1-(4-(2-(2-aminopyridin-3-yl)-6-phenyl-3H-imidazo[4,5- | 579.2 | + |
| | b]pyridin-3-yl)benzyl)piperidin-4-yl)oxy)nicotinonitrile | | |
| 13 | 4-(4-(4-(2-(2-aminopyridin-3-yl)-3H-imidazo[4,5-b]pyridin-3- | 488.1 | + |
| | yl)benzyl)piperazin-1-yl)picolinonitrile | | |
| 14 | 5-(4-(4-(2-(2-aminopyridin-3-yl)-3H-imidazo[4,5-b]pyridin-3- | 488.2 | + |
| | yl)benzyl)piperazin-1-yl)picolinonitrile | | |
| 15 | 6-(4-(4-(2-(2-aminopyridin-3-yl)-3H-imidazo[4,5-b]pyridin-3- | 488.3 | + |
| | yl)benzyl)piperazin-1-yl)picolinonitrile | | |
| 16 | 5-(4-(4-(2-(2-aminopyridin-3-yl)-3H-imidazo[4,5-b]pyridin-3- | 488.3 | + |
| | yl)benzyl)piperazin-1-yl)nicotinonitrile | | |
| 17 | 2-(4-(4-(2-(2-aminopyridin-3-yl)-3H-imidazo[4,5-b]pyridin-3- | 488.3 | + |
| | yl)benzyl)piperazin-1-yl)isonicotinonitrile | | |
| 18 | 4-(4-(4-(2-(2-aminopyridin-3-yl)-3H-imidazo[4,5-b]pyridin-3- | 489.4 | ++ |
| | yl)benzyl)piperazin-1-yl)pyrimidine-2-carbonitrile | | |
| 19 | 6-(4-(4-(2-(2-aminopyridin-3-yl)-3H-imidazo[4,5-b]pyridin-3- | 489.3 | + |
| | yl)benzyl)piperazin-1-yl)pyrazine-2-carbonitrile | | |
| 20 | 2-(4-(4-(2-(2-aminopyridin-3-yl)-3H-imidazo[4,5-b]pyridin-3- | 489.3 | + |
| | yl)benzyl)piperazin-1-yl)pyrimidine-4-carbonitrile | | |
| 21 | 6-(4-(4-(2-(2-aminopyridin-3-yl)-3H-imidazo[4,5-b]pyridin-3- | 489.3 | ++ |
| | yl)benzyl)piperazin-1-yl)pyrimidine-4-carbonitrile | | |
| 22 | 6-(4-(4-(2-(2-aminopyridin-3-yl)-3H-imidazo[4,5-b]pyridin-3- | 489.1 | + |
| | yl)benzyl)piperazin-1-yl)pyridazine-4-carbonitrile | | |
| 23 | N-(1-(4-(2-(2-aminopyridin-3-yl)-3H-imidazo[4,5-b]pyridin-3- | 530.2 | + |
| | yl)benzyl)piperidin-4-yl)-2-cyanoisonicotinamide | | |
| 24 | N-(1-(4-(2-(2-aminopyridin-3-yl)-3H-imidazo[4,5-b]pyridin-3- | 530.2 | + |
| | yl)benzyl)piperidin-4-yl)-6-cyanonicotinamide | | |
| 25 | N-(1-(4-(2-(2-aminopyridin-3-yl)-3H-imidazo[4,5-b]pyridin-3- | 530.1 | + |
| | yl)benzyl)piperidin-4-yl)-5-cyanopicolinamide | | |
| 26 | N-(1-(4-(2-(2-aminopyridin-3-yl)-3H-imidazo[4,5-b]pyridin-3- | 530.2 | + |
| | yl)benzyl)piperidin-4-yl)-5-cyanonicotinamide | | |
| 27 | N-(1-(4-(2-(2-aminopyridin-3-yl)-3H-imidazo[4,5-b]pyridin-3- | 530.3 | + |
| | yl)benzyl)piperidin-4-yl)-2-cyanonicotinamide | | |
| 28 | N-(1-(4-(2-(2-aminopyridin-3-yl)-3H-imidazo[4,5-b]pyridin-3- | 529.9 | + |
| | yl)benzyl)piperidin-4-yl)-3-cyanopicolinamide | | |
| 29 | N-(1-(4-(2-(2-aminopyridin-3-yl)-3H-imidazo[4,5-b]pyridin-3- | 530.1 | + |
| | yl)benzyl)piperidin-4-yl)-4-cyanonicotinamide | | |
| 30 | N-(1-(4-(2-(2-aminopyridin-3-yl)-3H-imidazo[4,5-b]pyridin-3- | 530.1 | + |
| | yl)benzyl)piperidin-4-yl)-6-cyanopicolinamide | | |
| 31 | N-(1-(4-(2-(2-aminopyridin-3-yl)-3H-imidazo[4,5-b]pyridin-3- | 530.1 | + |
| | yl)benzyl)piperidin-4-yl)-4-cyanopicolinamide | | |
| 32 | N-(1-(4-(2-(2-aminopyridin-3-yl)-3H-imidazo[4,5-b]pyridin-3- | 531.0 | ++ |
| | yl)benzyl)piperidin-4-yl)-2-cyanopyrimidine-4-carboxamide | | |
| 33 | N-(1-(4-(2-(2-aminopyridin-3-yl)-3H-imidazo[4,5-b]pyridin-3- | 531.1 | + |
| | yl)benzyl)piperidin-4-yl)-6-cyanopyrimidine-4-carboxamide | | |
| 34 | N-(1-(4-(2-(2-aminopyridin-3-yl)-3H-imidazo[4,5-b]pyridin-3- | 586.2 | ++ |
| | yl)benzyl)piperidin-4-yl)-2-cyanobenzo[d]thiazole-5- | | |
| | carboxamide | | |
| 35 | N-(1-(4-(2-(2-aminopyridin-3-yl)-3H-imidazo[4,5-b]pyridin-3- | 604.3 | ++ |
| | yl)benzyl)piperidin-4-yl)-2-cyano-5-fluorobenzo[d]thiazole-7- | | |
| | carboxamide | | |
| 36 | N-(1-(4-(2-(2-aminopyridin-3-yl)-3H-imidazo[4,5-b]pyridin-3- | 530.2 | + |
| | yl)benzyl)piperidin-4-yl)-3-cyanoisonicotinamide | | |
| 37 | N-(3-(2-(2-aminopyridin-3-yl)-3-(4-((4-(2-cyanopyridin-4- | 621.2 | +++ |
| | yl)piperazin-1-yl)methyl)phenyl)-3H-imidazo[4,5-b]pyridin-5- | | |
| | yl)phenyl)acetamide | | |
| 38 | N-(3-(2-(2-aminopyridin-3-yl)-3-(4-((4-(6-cyanopyridin-3- | 621.2 | ++ |
| | yl)piperazin-1-yl)methyl)phenyl)-3H-imidazo[4,5-b]pyridin-5- | | |
| | yl)phenyl)acetamide | | |
| 39 | N-(3-(2-(2-aminopyridin-3-y)-3-(4-(4-(6-cyanopyridin-2- | 621.2 | ++ |
| | yl)piperazin-1-yl)methyl)phenyl)-3H-imidazo[4,5-b]pyridin-5- | | |
| | yl)phenyl)acetamide | | |
| 40 | N-(3-(2-(2-aminopyridin-3-yl)-3-(4-((4-(6-cyanopyrazin-2- | 622.2 | ++++ |
| | yl)piperazin-1-yl)methyl)phenyl)-3H-imidazo[4,5-b]pyridin-5- | | |
| | yl)phenyl)acetamide | | |
| 41 | N-(3-(2-(2-aminopyridin-3-yl)-3-(4-((4-(6-cyanopyrimidin-4- | 622.2 | ++++ |
| | yl)piperazin-1-yl)methyl)phenyl)-3H-imidazo[4,5-b]pyridin-5- | | |
| | yl)phenyl)acetamide | | |
| 42 | N-(3-(2-(2-aminopyridin-3-yl)-3-(4-((4-(2-cyanopyrimidin-5- | 622.3 | +++ |
| | yl)piperazin-1-yl)methyl)phenyl)-3H-imidazo[4,5-b]pyridin-5- | | |
| | yl)phenyl)acetamide | | |
| 43 | N-(3-(2-(2-aminopyridin-3-yl)-3-(4-((4-(6-cyanopyridazin-4- | 622.4 | ++ |
| | yl)piperazin-1-yl)methyl)phenyl)-3H-imidazo[4,5-b]pyridin-5- | | |
| | yl)phenyl)acetamide | | |
| 44 | N-(3-(2-(2-aminopyridin-3-yl)-3-(4-((4-(2-cyanopyrimidin-4- | 622.5 | ++++ |
| | yl)piperazin-1-yl)methyl)phenyl)-3H-imidazo[4,5-b]pyridin-5- | | |
| | yl)phenyl)acetamide | | |
| 45 | N-(3-(2-(2-aminopyridin-3-yl)-3-(4-((4-(5-cyanopyridazin-3- | 622.2 | ++ |
| | yl)piperazin-1-yl)methyl)phenyl)-3H-imidazo[4,5-b]pyridin-5- | | |
| | yl)phenyl)acetamide | | |
| 46 | N-(3-(2-(2-aminopyridin-3-yl)-3-(4-((4-(4-cyanopyrimidin-2- | 622.3 | ++++ |
| | yl)piperazin-1-yl)methyl)phenyl)-3H-imidazo[4,5-b]pyridin-5- | | |
| | yl)phenyl)acetamide | | |
| 47 | 4-((4-(2-(2-aminopyridin-3-yl)-3H-imidazo[4,5-b]pyridin-3- | 420.3 | + |
| | yl)benzyl)amino)pyrimidine-2-carbonitrile | | |
| 48 | 4-((4-(2-(2-aminopyridin-3-yl)-5-phenyl-3H-imidazo[4,5- | 496.4 | +++ |
| | b]pyridin-3-yl)benzyl)amino)pyrimidine-2-carbonitrile | | |
| 49 | 4-(4-(4-(2-(2-aminopyridin-3-yl)-5-phenyl-3H-imidazo[4,5- | 565.4 | ++++ |
| | b]pyridin-3-yl)benzyl)piperazin-1-yl)pyrimidine-2-carbonitrile | | |
| 50 | 6-((4-(2-(2-aminopyridin-3-yl)-3H-imidazo[4,5-b]pyridin-3- | 420.1 | + |
| | yl)benzyl)amino)pyridazine-4-carbonitrile | | |
| 51 | 6-((1-(4-(2-(2-aminopyridin-3-yl)-3H-imidazo[4,5-b]pyridin-3- | 503.2 | + |
| | yl)benzyl)piperidin-4-yl)amino)pyridazine-4-carbonitrile | | |
| 52 | 6-((4-(2-(2-aminopyridin-3-yl)-5-phenyl-3H-imidazo[4,5- | 496.1 | ++ |
| | b]pyridin-3-yl)benzyl)amino)pyridazine-4-carbonitrile | | |
| 53 | 6-(4-(4-(2-(2-aminopyridin-3-yl)-5-phenyl-3H-imidazo[4,5- | 565.2 | ++ |
| | b]pyridin-3-yl)benzyl)piperazin-1-yl)pyridazine-4-carbonitrile | | |
| 54 | 6-((1-(4-(2-(2-aminopyridin-3-yl)-5-phenyl-3H-imidazo[4,5- | 579.3 | ++ |
| | b]pyridin-3-yl)benzyl)piperidin-4-yl)amino)pyridazine-4- | | |
| | carbonitrile | | |
| 55 | N-(1-(4-(2-(2-aminopyridin-3-yl)-5-phenyl-3H-imidazo[4,5- | 606.2 | +++ |
| | b]pyridin-3-yl)benzyl)piperidin-4-yl)-2-cyanoisonicotinamide | | |
| 56 | N-(1-(4-(2-(2-aminopyridin-3-yl)-5-phenyl-3H-imidazo[4,5- | 606.2 | ++++ |
| | b]pyridin-3-yl)benzyl)piperidin-4-yl)-6-cyanopicolinamide | | |
| 57 | N-(1-(4-(2-(2-aminopyridin-3-yl)-5-phenyl-3H-imidazo[4,5- | 606.2 | ++ |
| | b]pyridin-3-yl)benzyl)piperidin-4-yl)-5-cyanonicotinamide | | |
| 58 | N-(1-(4-(2-(2-aminopyridin-3-yl)-5-phenyl-3H-imidazo[4,5- | 606.4 | ++++ |
| | b]pyridin-3-yl)benzyl)piperidin-4-yl)-2-cyanonicotinamide | | |
| 59 | N-(1-(4-(2-(2-aminopyridin-3-yl)-5-phenyl-3H-imidazo[4,5- | 606.2 | +++ |
| | b]pyridin-3-yl)benzyl)piperidin-4-yl)-3-cyanoisonicotinamide | | |
| 60 | N-(1-(4-(2-(2-aminopyridin-3-yl)-5-phenyl-3H-imidazo[4,5- | 606.3 | ++++ |
| | b]pyridin-3-yl)benzyl)piperidin-4-yl)-4-cyanonicotinamide | | |
| 61 | N-(1-(4-(2-(2-aminopyridin-3-yl)-5-phenyl-3H-imidazo[4,5- | 606.1 | ++++ |
| | b]pyridin-3-yl)benzyl)piperidin-4-yl)-3-cyanopicolinamide | | |
| 62 | 4-((1-(4-(2-(2-aminopyridin-3-yl)-3H-imidazo[4,5-b]pyridin-3- | 502.4 | + |
| | yl)benzyl)piperidin-4-yl)amino)picolinonitrile | | |
| 63 | 6-((1-(4-(2-(2-aminopyridin-3-yl)-3H-imidazo[4,5-b]pyridin-3- | 502.2 | + |
| | yl)benzyl)piperidin-4-yl)amino)picolinonitrile | | |
| 64 | 6-((1-(4-(2-(2-aminopyridin-3-yl)-3H-imidazo[4,5-b]pyridin-3- | 503.1 | + |
| | yl)benzyl)piperidin-4-yl)amino)pyrimidine-4-carbonitrile | | |
| 65 | 5-(4-(4-(2-(2-aminopyridin-3-yl)-5-phenyl-3H-imidazo[4,5- | 564.1 | ++ |
| | b]pyridin-3-yl)benzyl)piperazin-1-yl)nicotinonitrile | | |
| 66 | 2-(4-(4-(2-(2-aminopyridin-3-yl)-5-phenyl-3H-imidazo[4,5- | 564.2 | + |
| | b]pyridin-3-yl)benzyl)piperazin-1-yl)isonicotinonitrile | | |
| 67 | 6-(4-(4-(2-(2-aminopyridin-3-yl)-5-phenyl-3H-imidazo[4,5- | 565.4 | ++ |
| | b]pyridin-3-yl)benzyl)piperazin-1-yl)pyrazine-2-carbonitrile | | |
| 68 | 2-(4-(4-(2-(2-aminopyridin-3-yl)-5-phenyl-3H-imidazo[4,5- | 565.2 | ++++ |
| | b]pyridin-3-yl)benzyl)piperazin-1-yl)pyrimidine-4-carbonitrile | | |
| 69 | 6-(4-(4-(2-(2-aminopyridin-3-yl)-3H-imidazo[4,5-b]pyridin-3- | 516.1 | + |
| | yl)benzyl)piperazine-1-carbonyl)picolinonitrile | | |
| 70 | 4-(4-(4-(2-(2-aminopyridin-3-yl)-5-phenyl-3H-imidazo[4,5- | 593.3 | ++ |
| | b]pyridin-3-yl)benzyl)piperazine-1-carbonyl)pyridazine-3- | | |
| | carbonitrile | | |
| 71 | N-(1-(4-(2-(2-aminopyridin-3-yl)-5-phenyl-3H-imidazo[4,5- | 607.4 | +++ |
| | b]pyridin-3-yl)benzyl)piperidin-4-yl)-3-cyanopyridazine-4- | | |
| | carboxamide | | |
| 72 | 4-(4-(4-(2-(2-aminopyridin-3-yl)-5-phenyl-3H-imidazo[4,5- | 593.4 | + |
| | b]pyridin-3-yl)benzyl)piperazine-1-carbonyl)pyrimidine-5- | | |
| | carbonitrile | | |
| 73 | N-(1-(4-(2-(2-aminopyridin-3-yl)-5-phenyl-3H-imidazo[4,5- | 607.3 | +++ |
| | b]pyridin-3-yl)benzyl)piperidin-4-yl)-5-cyanopyrimidine-4- | | |
| | carboxamide | | |
| 74 | 5-((1-(4-(2-(2-aminopyridin-3-yl)-3H-imidazo[4,5-b]pyridin-3- | 502.4 | + |
| | yl)benzyl)piperidin-4-yl)amino)nicotinonitrile | | |
| 75 | 2-((1-(4-(2-(2-aminopyridin-3-yl)-3H-imidazo[4,5-b]pyridin-3- | 502.1 | + |
| | yl)benzyl)piperidin-4-yl)amino)isonicotinonitrile | | |
| 76 | 6-((1-(4-(2-(2-aminopyridin-3-yl)-3H-imidazo[4,5-b]pyridin-3- | 503.2 | + |
| | yl)benzyl)piperidin-4-yl)amino)pyrazine-2-carbonitrile | | |
| 77 | 2-((1-(4-(2-(2-aminopyridin-3-yl)-3H-imidazo[4,5-b]pyridin-3- | 503.3 | ++ |
| | yl)benzyl)piperidin-4-yl)amino)pyrimidine-4-carbonitrile | | |
| 78 | 6-(4-(4-(2-(2-aminopyridin-3-yl)-5-phenyl-3H-imidazo[4,5- | 565.2 | ++++ |
| | b]pyridin-3-yl)benzyl)piperazin-1-yl)pyrimidine-4-carbonitrile | | |
| 79 | 5-(4-(4-(2-(2-aminopyridin-3-yl)-3H-imidazo[4,5-b]pyridin-3- | 516.2 | + |
| | yl)benzyl)piperazine-1-carbonyl)nicotinonitrile | | |
| 80 | 6-(4-(4-(2-(2-aminopyridin-3-yl)-3H-imidazo[4,5-b]pyridin-3- | 516.2 | + |
| | yl)benzyl)piperazine-1-carbonyl)nicotinonitrile | | |
| 81 | N-(1-(4-(2-(2-aminopyridin-3-yl)-5-phenyl-3H-imidazo[4,5- | 606.4 | ++ |
| | b]pyridin-3-yl)benzyl)piperidin-4-yl)-5-cyanopicolinamide | | |
| 82 | 4-(4-(4-(2-(2-aminopyridin-3-yl)-5-phenyl-3H-imidazo[4,5- | 564.4 | +++ |
| | b]pyridin-3-yl)benzyl)piperazin-1-yl)picolinonitrile | | |
| 83 | 6-(4-(4-(2-(2-aminopyridin-3-yl)-5-phenyl-3H-imidazo[4,5- | 564.3 | ++ |
| | b]pyridin-3-yl)benzyl)piperazin-1-yl)picolinonitrile | | |
| 84 | 5-((1-(4-(2-(2-aminopyridin-3-yl)-5-phenyl-3H-imidazo[4,5- | 578.2 | +++ |
| | b]pyridin-3-yl)benzyl)piperidin-4-yl)amino)picolinonitrile | | |
| 85 | 4-((1-(4-(2-(2-aminopyridin-3-yl)-5-phenyl-3H-imidazo[4,5- | 578.2 | ++++ |
| | b]pyridin-3-yl)benzyl)piperidin-4-yl)amino)picolinonitrile | | |
| 86 | 6-((1-(4-(2-(2-aminopyridin-3-yl)-5-phenyl-3H-imidazo[4,5- | 578.2 | ++ |
| | b]pyridin-3-yl)benzyl)piperidin-4-yl)amino)picolinonitrile | | |
| 87 | 2-((1-(4-(2-(2-aminopyridin-3-yl)-5-phenyl-3H-imidazo[4,5- | 578.3 | ++ |
| | b]pyridin-3-yl)benzyl)piperidin-4-yl)amino)isonicotinonitrile | | |
| 88 | 4-((1-(4-(2-(2-aminopyridin-3-yl)-5-phenyl-3H-imidazo[4,5- | 579.4 | ++++ |
| | b]pyridin-3-yl)benzyl)piperidin-4-yl)amino)pyrimidine-2- | | |
| | carbonitrile | | |
| 89 | 6-((1-(4-(2-(2-aminopyridin-3-yl)-5-phenyl-3H-imidazo[4,5- | 579.1 | ++++ |
| | b]pyridin-3-yl)benzyl)piperidin-4-yl)amino)pyrazine-2- | | |
| | carbonitrile | | |
| 90 | 2-((1-(4-(2-(2-aminopyridin-3-yl)-5-phenyl-3H-imidazo[4,5- | 579.1 | ++++ |
| | b]pyridin-3-yl)benzyl)piperidin-4-yl)amino)pyrimidine-4- | | |
| | carbonitrile | | |
| 91 | 6-((1-(4-(2-(2-aminopyridin-3-yl)-5-phenyl-3H-imidazo[4,5- | 579.2 | ++++ |
| | b]pyridin-3-yl)benzyl)piperidin-4-yl)amino)pyrimidine-4- | | |
| | carbonitrile | | |
| 92 | 4-((1-(4-(2-(2-aminopyridin-3-yl)-3H-imidazo[4,5-b]pyridin-3- | 503.3 | +++ |
| | yl)benzyl)piperidin-4-yl)amino)pyrimidine-2-carbonitrile | | |
| 93 | 5-((1-(4-(2-(2-aminopyridin-3-yl)-5-phenyl-3H-imidazo[4,5- | 578.4 | ++ |
| | b]pyridin-3-yl)benzyl)piperidin-4-yl)amino)nicotinonitrile | | |
| 94 | 5-(4-(4-(2-(2-aminopyridin-3-yl)-5-phenyl-3H-imidazo[4,5- | 564.0 | ++ |
| | b]pyridin-3-yl)benzyl)piperazin-1-yl)picolinonitrile | | |
| 95 | 5-((4-(2-(2-aminopyridin-3-yl)-3H-imidazo[4,5-b]pyridin-3- | 420.1 | + |
| | yl)benzyl)amino)pyrimidine-2-carbonitrile | | |
| 96 | 5-((4-(2-(2-aminopyridin-3-yl)-5-phenyl-3H-imidazo[4,5- | 496.3 | ++++ |
| | b]pyridin-3-yl)benzyl)amino)pyrimidine-2-carbonitrile | | |
| 97 | 5-(4-(4-(2-(2-aminopyridin-3-yl)-5-phenyl-3H-imidazo[4,5- | 565.3 | +++ |
| | b]pyridin-3-yl)benzyl)piperazin-1-yl)pyrimidine-2-carbonitrile | | |
| 98 | N-(4-(2-(2-aminopyridin-3-yl)-3H-imidazo[4,5-b]pyridin-3- | 447.2 | + |
| | yl)benzyl)-2-cyanonicotinamide | | |
| 99 | N-(4-(2-(2-aminopyridin-3-yl)-3H-imidazo[4,5-b]pyridin-3- | 447.0 | + |
| | yl)benzyl)-3-cyanoisonicotinamide | | |
| 100 | N-(4-(2-(2-aminopyridin-3-yl)-3H-imidazo[4,5-b]pyridin-3- | 447.1 | + |
| | yl)benzyl)-4-cyanonicotinamide | | |
| 101 | N-(4-(2-(2-aminopyridin-3-yl)-3H-imidazo[4,5-b]pyridin-3- | 447.1 | + |
| | yl)benzyl)-3-cyanopicolinamide | | |
| 102 | 5-(4-(4-(2-(2-aminopyridin-3-yl)-3H-imidazo[4,5-b]pyridin-3- | 516.2 | + |
| | yl)benzyl)piperazine-1-carbonyl)picolinonitrile | | |
| 103 | N-(4-(2-(2-aminopyridin-3-yl)-5-phenyl-3H-imidazo[4,5- | 523.2 | + |
| | b]pyridin-3-yl)benzyl)-2-cyanonicotinamide | | |
| 104 | N-(4-(2-(2-aminopyridin-3-yl)-5-phenyl-3H-imidazo[4,5- | 523.2 | + |
| | b]pyridin-3-yl)benzyl)-3-cyanoisonicotinamide | | |
| 105 | N-(4-(2-(2-aminopyridin-3-yl)-5-phenyl-3H-imidazo[4,5- | 523.2 | + |
| | b]pyridin-3-yl)benzyl)-4-cyanonicotinamide | | |
| 106 | N-(4-(2-(2-aminopyridin-3-yl)-6-phenyl-3H-imidazo[4,5- | 523.2 | + |
| | b]pyridin-3-yl)benzyl)-6-cyanonicotinamide | | |
| 107 | N-(1-(4-(2-(2-aminopyridin-3-yl)-6-phenyl-3H-imidazof4,5- | 606.3 | + |
| | b]pyridin-3-yl)benzyl)piperidin-4-yl)-2-cyanoisonicotinamide | | |
| 108 | N-(4-(2-(2-aminopyridin-3-yl)-3H-imidazo[4,5-b]pyridin-3- | 447.1 | + |
| | yl)benzyl)-6-cyanopicolinamide | | |
| 109 | 4-(4-(4-(2-(2-aminopyridin-3-yl)-3H-imidazo[4,5-b]pyridin-3- | 516.3 | + |
| | yl)benzyl)piperazine-1-carbonyl)picolinonitrile | | |
| 110 | 2-(4-(4-(2-(2-aminopyridin-3-yl)-3H-imidazo[4,5-b]pyridin-3- | 516.0 | + |
| | yl)benzyl)piperazine-1-carbonyl)nicotinonitrile | | |
| 111 | 5-(4-(4-(2-(2-aminopyridin-3-yl)-6-phenyl-3H-imidazo[4,5- | 592.1 | + |
| | b]pyridin-3-yl)benzyl)piperazine-1-carbonyl)picolinonitrile | | |
| 112 | 5-((4-(2-(2-aminopyridin-3-yl)-5-phenyl-3H-imidazo[4,5- | 496.2 | +++ |
| | b]pyridin-3-yl)benzyl)amino)pyridazine-3-carbonitrile | | |
| 113 | N-(4-(2-(2-aminopyridin-3-yl)-3H-imidazo[4,5-b]pyridin-3- | 448.2 | + |
| | yl)benzyl)-6-cyanopyrimidine-4-carboxamide | | |
| 114 | 6-(4-(4-(2-(2-aminopyridin-3-yl)-3H-imidazo[4,5-b]pyridin-3- | 517.1 | ++ |
| | yl)benzyl)piperazine-1-carbonyl)pyrimidine-4-carbonitrile | | |
| 115 | N-(4-(2-(2-aminopyridin-3-yl)-5-phenyl-3H-imidazo[4,5- | 523.9 | ++++ |
| | b]pyridin-3-yl)benzyl)-6-cyanopyrimidine-4-carboxamide | | |
| 116 | 6-(4-(4-(2-(2-aminopyridin-3-yl)-5-phenyl-3H-imidazo[4,5- | 593.2 | ++++ |
| | b]pyridin-3-yl)benzyl)piperazine-1-carbonyl)pyrimidine-4- | | |
| | carbonitrile | | |
| 117 | N-(1-(4-(2-(2-aminopyridin-3-yl)-5-phenyl-3H-imidazo[4,5- | 607.2 | +++ |
| | b]pyridin-3-yl)benzyl)piperidin-4-yl)-6-cyanopyrimidine-4- | | |
| | carboxamide | | |
| 118 | N-(4-(2-(2-aminopyridin-3-yl)-5-phenyl-3H-imidazo[4,5- | 523.3 | + |
| | b]pyridin-3-yl)benzyl)-3-cyanopicolinamide | | |
| 119 | N-(4-(2-(2-aminopyridin-3-yl)-3H-imidazo[4,5-b]pyridin-3- | 448.3 | + |
| | yl)benzyl)-2-cyanopyrimidine-5-carboxamide | | |
| 120 | N-(4-(2-(2-aminopyridin-3-yl)-5-phenyl-3H-imidazo[4,5- | 524.3 | ++++ |
| | b]pyridin-3-yl)benzyl)-2-cyanopyrimidine-5-carboxamide | | |
| 121 | N-(4-(2-(2-aminopyridin-3-yl)-3H-imidazo[4,5-b]pyridin-3- | 447.3 | + |
| | yl)benzyl)-6-cyanonicotinamide | | |
| 122 | N-(4-(2-(2-aminopyridin-3-yl)-3H-imidazo[4,5-b]pyridin-3- | 447.1 | + |
| | yl)benzyl)-2-cyanoisonicotinamide | | |
| 123 | N-(4-(2-(2-aminopyridin-3-yl)-3H-imidazo[4,5-b]pyridin-3- | 447.0 | + |
| | yl)benzyl)-5-cyanonicotinamide | | |
| 124 | N-(4-(2-(2-aminopyridin-3-yl)-3H-imidazo[4,5-b]pyridin-3- | 447.0 | + |
| | yl)benzyl)-5-cyanopicolinamide | | |
| 125 | N-(4-(2-(2-aminopyridin-3-yl)-5-phenyl-3H-imidazo[4,5- | 523.4 | +++ |
| | bjpyridin-3-yl)benzyl)-5-cyanonicotinamide | | |
| 126 | N-(4-(2-(2-aminopyridin-3-yl)-5-phenyl-3H-imidazo[4,5- | 523.4 | + |
| | b]pyridin-3-yl)benzyl)-5-cyanopicolinamide | | |
| 127 | 4-((4-(2-(2-aminopyridin-3-yl)-3H-imidazo[4,5-b]pyridin-3- | 419.0 | + |
| | yl)benzyl)amino)picolinonitrile | | |
| 128 | 5-((4-(2-(2-aminopyridin-3-yl)-3H-imidazo[4,5-b]pyridin-3- | 419.1 | + |
| | yl)benzyl)amino)picolinonitrile | | |
| 129 | 4-((4-(2-(2-aminopyridin-3-yl)-5-phenyl-3H-imidazo[4,5- | 495.3 | ++ |
| | b]pyridin-3-yl)benzyl)amino)picolinonitrile | | |
| 130 | 6-((4-(2-(2-aminopyridin-3-yl)-3H-imidazo[4,5-b]pyridin-3- | 419.1 | + |
| | yl)benzyl)amino)picolinonitrile | | |
| 131 | 5-((4-(2-(2-aminopyridin-3-yl)-3H-imidazo[4,5-b]pyridin-3- | 419.1 | + |
| | yl)benzyl)amino)nicotinonitrile | | |
| 132 | 2-((4-(2-(2-aminopyridin-3-yl)-3H-imidazo[4,5-b]pyridin-3- | 419.1 | + |
| | yl)benzyl)amino)isonicotinonitrile | | |
| 133 | 6-((4-(2-(2-aminopyridin-3-yl)-3H-imidazo[4,5-b]pyridin-3- | 420.1 | + |
| | yI)benzyl)amino)pyrazine-2-carbonitrile | | |
| 134 | 2-((4-(2-(2-aminopyridin-3-yl)-3H-imidazo[4,5-b]pyridin-3- | 420.0 | + |
| | yl)benzyl)amino)pyrimidine-4-carbonitrile | | |
| 135 | 6-((4-(2-(2-aminopyridin-3-yl)-3H-imidazo[4,5-b]pyridin-3- | 420.0 | + |
| | yl)benzyl)amino)pyrimidine-4-carbonitrile | | |
| 136 | 6-((4-(2-(2-aminopyridin-3-yl)-5-phenyl-3H-imidazo[4,5- | 496.1 | +++ |
| | b]pyridin-3-yl)benzyl)amino)pyrimidine-4-carbonitrile | | |
| 137 | 4-(4-(4-(2-(2-aminopyridin-3-yl)-6-phenyl-3H-imidazo[4,5- | 592.3 | + |
| | b]pyridin-3-yl)benzyl)piperazine-1-carbonyl)picolinonitrile | | |
| 138 | 6-(4-(4-(2-(2-aminopyridin-3-yl)-5-phenyl-3H-imidazo[4,5- | 592.1 | ++ |
| | b]pyridin-3-yl)benzyl)piperazine-1-carbonyl)picolinonitrile | | |
| 139 | 5-14-14-(2-(2-aminopyridin-3-yl)-5-phenyl-3H-imidazo[4,5- | 592.4 | + |
| | b]pyridin-3-yI)benzyl)piperazine-1-carbonyl)nicotinonitrile | | |
| 140 | 2-(4-(4-(2-(2-aminopyridin-3-yl)-5-phenyl-3H-imidazo[4,5- | 592.1 | ++ |
| | b]pyridin-3-yl)benzyl)piperazine-1-carbonyl)isonicotinonitrile | | |
| 141 | 4-(4-(4-(2-(2-aminopyridin-3-yl)-5-phenyl-3H-imidazo[4,5- | 593.1 | ++++ |
| | b]pyridin-3-yl)benzyl)piperazine-1-carbonyl)pyrimidine-2- | | |
| | carbonitrile | | |
| 142 | 6-(4-(4-(2-(2-aminopyridin-3-yl)-5-phenyl-3H-imidazo[4,5- | 592.2 | + |
| | b]pyridin-3-yl)benzyl)piperazine-1-carbonyl)nicotinonitrile | | |
| 143 | 3-(4-(4-(2-(2-aminopyridin-3-yl)-5-phenyl-3H-imidazo[4,5- | 592.2 | ++ |
| | b]pyridin-3-yl)benzyl)piperazine-1-carbonyl)picolinonitrile | | |
| 144 | 4-(4-(4-(2-(2-aminopyridin-3-yl)-5-phenyl-3H-imidazo[4,5- | 592.3 | + |
| | b]pyridin-3-yl)benzyl)piperazine-1-carbonyl)nicotinonitrile | | |
| 145 | 3-(4-(4-(2-(2-aminopyridin-3-yl)-5-phenyl-3H-imidazo[4,5- | 592.2 | + |
| | b]pyridin-3-yl)benzyl)piperazine-1-carbonyl)isonicotinonitrile | | |
| 146 | 2-(4-(4-(2-(2-aminopyridin-3-yl)-5-phenyl-3H-imidazo[4,5- | 592.2 | + |
| | b]pyridin-3-yl)benzyl)piperazine-1-carbonyl)nicotinonitrile | | |
| 147 | N-(4-(2-(2-aminopyridin-3-yl)-3H-imidazo[4,5-b]pyridin-3- | 447.3 | + |
| | yl)benzyl)-4-cyanopicolinamide | | |
| 148 | N-(4-(2-(2-aminopyridin-3-yl)-3H-imidazo[4,5-b]pyridin-3- | 448.1 | + |
| | yl)benzyl)-2-cyanopyrimidine-4-carboxamide | | |
| 149 | 2-(4-(4-(2-(2-aminopyridin-3-yl)-3H-imidazo[4,5-b]pyridin-3- | 516.1 | + |
| | yl)benzyl)piperazine-1-carbonyl)isonicotinonitrile | | |
| 150 | N-(1-(4-(2-(2-aminopyridin-3-yl)-5-phenyl-3H-imidazo[4,5- | 606.1 | ++ |
| | b]pyridin-3-yl)benzyl)piperidin-4-yl)-4-cyanopicolinamide | | |
| 151 | N-(1-(4-(2-(2-aminopyridin-3-yl)-5-phenyl-3H-imidazo[4,5- | 607.1 | ++++ |
| | b]pyridin-3-yl)benzyl)piperidin-4-yl)-2-cyanopyrimidine-4- | | |
| | carboxamide | | |
| 152 | 4-(4-(4-(2-(2-aminopyridin-3-yl)-3H-imidazo[4,5-b]pyridin-3- | 517.2 | ++ |
| | yl)benzyl)piperazine-1-carbonyl)pyrimidine-2-carbonitrile | | |
| 153 | 3-(4-(4-(2-(2-aminopyridin-3-yl)-3H-imidazo[4,5-b]pyridin-3- | 516.4 | + |
| | yl)benzyl)piperazine-1-carbonyl)picolinonitrile | | |
| 154 | 4-(4-(4-(2-(2-aminopyridin-3-yl)-3H-imidazo[4,5-b]pyridin-3- | 516.3 | + |
| | yl)benzyl)piperazine-1-carbonyl)nicotinonitrile | | |
| 155 | 3-(4-(4-(2-(2-aminopyridin-3-yl)-3H-imidazo[4,5-b]pyridin-3- | 516.2 | + |
| | yl)benzyl)piperazine-1-carbonyl)isonicotinonitrile | | |
| 156 | 4-((1-(4-(2-(2-aminopyridin-3-yl)-5-phenyl-3H-imidazo[4,5- | 593.4 | +++ |
| | b]pyridin-3-yl)benzyl)piperidin-4- | | |
| | yl)(methyl)amino)pyrimidine-2-carbonitrile | | |
| 157 | 5-((1-(4-(2-(2-aminopyridin-3-yl)-3H-imidazo[4,5-b]pyridin-3- | 502.1 | + |
| | yl)benzyl)piperidin-4-yl)amino)picolinonitrile | | |
| 158 | 5-((1-(4-(2-(2-aminopyridin-3-yl)-5-phenyl-3H-imidazo[4,5- | 579.4 | +++ |
| | b]pyridin-3-yl)benzyl)piperidin-4-yl)amino)pyridazine-3- | | |
| | carbonitrile | | |
| 159 | 5-(4-(4-(2-(2-aminopyridin-3-yl)-5-phenyl-3H-imidazo[4,5- | 565.4 | +++ |
| | b]pyridin-3-yl)benzyl)piperazin-1-yl)pyridazine-3-carbonitrile | | |
| 160 | N-(4-(2-(2-aminopyridin-3-yl)-5-phenyl-3H-imidazo[4,5- | 523.2 | +++ |
| | b]pyridin-3-yl)benzyl)-6-cyanonicotinamide | | |
| 161 | N-(4-(2-(2-aminopyridin-3-yl)-5-phenyl-3H-imidazo[4,5- | 523.2 | +++ |
| | b]pyridin-3-yl)benzyl)-2-cyanoisonicotinamide | | |
| 162 | N-(4-(2-(2-aminopyridin-3-yl)-5-phenyl-3H-imidazo[4,5- | 523.2 | ++ |
| | b]pyridin-3-yl)benzyl)-6-cyanopicolinamide | | |
| 163 | 3-(4-(4-(2-(2-aminopyridin-3-yl)-5-phenyl-3H-imidazo[4,5- | 593.3 | ++ |
| | b]pyridin-3-yl)benzyl)piperazine-1-carbonyl)pyrazine-2- | | |
| | carbonitrile | | |
| 164 | 3-(4-(4-(2-(2-aminopyridin-3-yl)-3H-imidazo[4,5-b]pyridin-3- | 517.3 | + |
| | yl)benzyl)piperazine-1-carbonyl)pyrazine-2-carbonitrile | | |
| 165 | N-(1-(4-(2-(2-aminopyridin-3-yl)-6-phenyl-3H-imidazo[4,5- | 606.2 | + |
| | b]pyridin-3-yl)benzyl)piperidin-4-yl)-6-cyanonicotinamide | | |
| 166 | 5-(4-(4-(2-(2-aminopyridin-3-yl)-5-phenyl-3H-imidazo[4,5- | 592.2 | +++ |
| | b]pyridin-3-yl)benzyl)piperazine-1-carbonyl)picolinonitrile | | |
| 167 | N-(1-(4-(2-(2-aminopyridin-3-yl)-5-phenyl-3H-imidazo[4,5- | 606.2 | ++ |
| | b]pyridin-3-yl)benzyl)piperidin-4-yl)-6-cyanonicotinamide | | |
| 168 | N-(4-(2-(2-aminopyridin-3-yl)-6-phenyl-3H-imidazo[4,5- | 523.2 | + |
| | b]pyridin-3-yl)benzyl)-6-cyanopicolinamide | | |
| 169 | N-(4-(2-(2-aminopyridin-3-yl)-6-phenyl-3H-imidazo[4,5- | 523.10 | ++ |
| | b]pyridin-3-yl)benzyl)-2-cyanoisonicotinamide | | |
| 170 | N-(4-(2-(2-aminopyridin-3-yl)-5-phenyl-3H-imidazo[4,5- | 523.1 | ++ |
| | b]pyridin-3-yl)benzyl)-4-cyanopicolinamide | | |
| 171 | N-(4-(2-(2-aminopyridin-3-yl)-5-phenyl-3H-imidazo[4,5- | 524.1 | +++ |
| | b]pyridin-3-yl)benzyl)-2-cyanopyrimidine-4-carboxamide | | |
| 172 | 4-(4-(4-(2-(2-aminopyridin-3-yl)-5-phenyl-3H-imidazo[4,5- | 592.2 | +++ |
| | b]pyridin-3-yl)benzyl)piperazine-1-carbonyl)picolinonitrile | | |
| 173 | N-(4-(2-(2-aminopyridin-3-yl)-3H-imidazo[4,5-b]pyridin-3- | 521.0 | ++++ |
| | yl)benzyl)-2-cyano-5-fluorobenzo[d]thiazole-7-carboxamide | | |
| 174 | N-(4-(2-(2-aminopyridin-3-yl)-3H-imidazo[4,5-b]pyridin-3- | 521.1 | +++ |
| | yl)benzyl)-2-cyano-6-fluorobenzo[d]thiazole-4-carboxamide | | |
| 175 | 7-(4-(4-(2-(2-aminopyridin-3-yl)-3H-imidazo[4,5-b]pyridin-3- | 590.3 | + |
| | yl)benzyl)piperazine-1-carbonyl)-5-fluorobenzo[d]thiazole-2- | | |
| | carbonitrile | | |
| 176 | N-(4-(2-(2-aminopyridin-3-yl)-5-phenyl-3H-imidazo[4,5- | 597.1 | +++ |
| | b]pyridin-3-yl)benzyl)-2-cyano-5-fluorobenzo[d]thiazole-7- | | |
| | carboxamide | | |
| 177 | N-(4-(2-(2-aminopyridin-3-yl)-5-phenyl-3H-imidazo[4,5- | 597.3 | ++++ |
| | b]pyridin-3-yl)benzyl)-2-cyano-6-fluorobenzo[d]thiazole-4- | | |
| | carboxamide | | |
| 178 | N-(4-(5-(3-acetamidophenyl)-2-(2-aminopyridin-3-yl)-3H- | 654.2 | ++++ |
| | imidazo[4,5-b]pyridin-3-yl)benzyl)-2-cyano-5- | | |
| | fluorobenzo[d]thiazole-7-carboxamide | | |
| 179 | N-(4-(5-(3-acetamidophenyl)-2-(2-aminopyridin-3-yl)-3H- | 654.4 | +++ |
| | imidazo[4,5-b]pyridin-3-yl)benzyl)-2-cyano-6- | | |
| | fluorobenzo[d]thiazole-4-carboxamide | | |
| 180 | 7-(4-(4-(2-(2-aminopyridin-3-yl)-5-phenyl-3H-imidazo[4,5- | 666.4 | +++ |
| | b]pyridin-3-yl)benzyl)piperazine-1-carbonyl)-5- | | |
| | fluorobenzo[d]thiazole-2-carbonitrile | | |
| 181 | 4-(4-(4-(2-(2-aminopyridin-3-yl)-5-phenyl-3H-imidazo[4,5- | 666.1 | +++ |
| | b]pyridin-3-yl)benzyl)piperazine-1-carbonyl)-6- | | |
| | fluorobenzo[d]thiazole-2-carbonitrile | | |
| 182 | N-(1-(4-(2-(2-aminopyridin-3-yl)-5-phenyl-3H-imidazo[4,5- | 680.2 | ++ |
| | b]pyridin-3-yl)benzyl)piperidin-4-yl)-2-cyano-5- | | |
| | fluorobenzo[d]thiazole-7-carboxamide | | |
| 183 | N-(1-(4-(2-(2-aminopyridin-3-yl)-5-phenyl-3H-imidazo[4,5- | 680.2 | ++ |
| | b]pyridin-3-yl)benzyl)piperidin-4-yl)-2-cyano-6- | | |
| | fluorobenzo[d]thiazole-4-carboxamide | | |
| 184 | 5-(4-(4-(2-(2-aminopyridin-3-yl)-5-phenyl-3H-imidazo[4,5- | 648.4 | ++++ |
| | b]pyridin-3-yl)benzyl)piperazine-1-carbonyl)benzo[d]thiazole- | | |
| | 2-carbonitrile | | |
| 185 | N-(1-(4-(2-(2-aminopyridin-3-yl)-5-phenyl-3H-imidazo[4,5- | 662.5 | ++++ |
| | b]pyridin-3-yl)benzyl)piperidin-4-yl)-2- | | |
| | cyanobenzo[d]thiazole-5-carboxamide | | |
| 186 | 6-((4-(2-(2-aminopyridin-3-yl)-5-phenyl-3H-imidazo[4,5- | 495.1 | ++ |
| | b]pyridin-3-yl)benzyl)amino)picolinonitrile | | |
| 187 | 5-((4-(2-(2-aminopyridin-3-yl)-5-phenyl-3H-imidazo[4,5- | 495.1 | ++ |
| | b]pyridin-3-yl)benzyl)amino)picolinonitrile | | |
| 188 | 5-((4-(2-(2-aminopyridin-3-yl)-5-phenyl-3H-imidazo[4,5- | 495.5 | ++ |
| | b]pyridin-3-yl)benzyl)amino)nicotinonitrile | | |
| 189 | 2-((4-(2-(2-aminopyridin-3-yl)-5-phenyl-3H-imidazo[4,5- | 495.2 | ++ |
| | b]pyridin-3-yl)benzyl)amino)isonicotinonitrile | | |
| 190 | 6-((4-(2-(2-aminopyridin-3-yI)-5-phenyl-3H-imidazo[4,5- | 496.3 | ++ |
| | b]pyridin-3-yl)benzyl)amino)pyrazine-2-carbonitrile | | |
| 191 | 2-((4-(2-(2-aminopyridin-3-yl)-5-phenyl-3H-imidazo[4,5- | 496.2 | +++ |
| | b]pyridin-3-yl)benzyl)amino)pyrimidine-4-carbonitrile | | |
| 192 | 4-((28,6S)-4-(4-(2-(2-aminopyridin-3-yl)-5-phenyl-3H- | 593.3 | +++ |
| | imidazo[4,5-b]pyridin-3-yl)benzyl)-2,6-dimethylpiperazin-1- | | |
| | yl)pyrimidine-2-carbonitrile | | |
| 193 | 2-((25,6S)-4-(4-(2-(2-aminopyridin-3-yl)-5-phenyl-3H- | 593.5 | ++ |
| | imidazo[4,5-b]pyridin-3-yl)benzyl)-2,6-dimethylpiperazin-1- | | |
| | yl)pyrimidine-4-carbonitrile | | |
| 194 | 6-((2S,6S)-4-(4-(2-(2-aminopyridin-3-yl)-5-phenyl-3H- | 593.3 | ++ |
| | imidazo[4,5-b]pyridin-3-yl)benzyl)-2,6-dimethylpiperazin-1- | | |
| | yl)pyrimidine-4-carbonitrile | | |
| 195 | 4-((2R,6R)-4-(4-(2-(2-aminopyridin-3-yl)-5-phenyl-3H- | 593.4 | ++++ |
| | imidazo[4,5-b]pyridin-3-yl)benzyl)-2,6-dimethylpiperazin-1- | | |
| | yl)pyrimidine-2-carbonitrile | | |
| 196 | 2-((2R,6R)-4-(4-(2-(2-aminopyridin-3-yl)-5-phenyl-3H- | 593.3 | ++ |
| | imidazo[4,5-b]pyridin-3-yl)benzyl)-2,6-dimethylpiperazin-1- | | |
| | yl)pyrimidine-4-carbonitrile | | |
| 197 | 6-((2R,6R)-4-(4-(2-(2-aminopyridin-3-yl)-5-phenyl-3H- | 593.4 | ++ |
| | imidazo[4,5-b]pyridin-3-yl)benzyl)-2,6-dimethylpiperazin-1- | | |
| | yl)pyrimidine-4-carbonitrile | | |
| 198 | 4-(7-(4-(2-(2-aminopyridin-3-yl)-5-phenyl-3H-imidazo[4,5- | 591.4 | +++ |
| | b]pyridin-3-yl)benzyl)-4,7-diazaspiro[2.5]octan-4- | | |
| | yl)pyrimidine-2-carbonitrile | | |
| 199 | 2-(7-(4-(2-(2-aminopyridin-3-yl)-5-phenyl-3H-imidazo[4,5- | 591.4 | ++ |
| | b]pyridin-3-yl)benzyl)-4,7-diazaspiro[2.5]octan-4- | | |
| | yl)pyrimidine-4-carbonitrile | | |
| 200 | 6-(7-(4-(2-(2-aminopyridin-3-yl)-5-phenyl-3H-imidazo[4,5- | 591.4 | +++ |
| | b]pyridin-3-yl)benzyl)-4,7-diazaspiro[2.5]octan-4- | | |
| | yl)pyrimidine-4-carbonitrile | | |
| 201 | 4-((3-(4-(2-(2-aminopyridin-3-yl)-5-phenyl-3H-imidazo[4,5- | 605.4 | ++ |
| | b]pyridin-3-yl)benzyl)-3-azabicyclo[3.2.1]octan-8- | | |
| | yl)amino)pyrimidine-2-carbonitrile | | |
| 202 | 6-((3-(4-(2-(2-aminopyridin-3-yl)-5-phenyl-3H-imidazo[4,5- | 605.3 | + |
| | b]pyridin-3-yl)benzyl)-3-azabicyclo[3.2.1]octan-8- | | |
| | yl)amino)pyrimidine-4-carbonitrile | | |
| 203 | 4-(3-(4-(2-(2-aminopyridin-3-yl)-5-phenyl-3H-imidazo[4,5- | 591.4 | ++ |
| | b]pyridin-3-yl)benzyl)-3,8-diazabicyclo[3.2.1]octan-8- | | |
| | yl)pyrimidine-2-carbonitrile | | |
| 204 | 2-(3-(4-(2-(2-aminopyridin-3-yl)-5-phenyl-3H-imidazo[4,5- | 591.5 | + |
| | b]pyridin-3-yl)benzyl)-3,8-diazabicyclo[3.2.1]octan-8- | | |
| | yl)pyrimidine-4-carbonitrile | | |
| 205 | 6-(3-(4-(2-(2-aminopyridin-3-yl)-5-phenyl-3H-imidazo[4,5- | 591.5 | ++ |
| | b]pyridin-3-yl)benzyl)-3,8-diazabicyclo[3.2.1]octan-8- | | |
| | yl)pyrimidine-4-carbonitrile | | |
| 206 | (S)-4-(4-(4-(2-(2-aminopyridin-3-yl)-5-phenyl-3H- | 579.4 | +++ |
| | imidazo[4,5-b]pyridin-3-yl)benzyl)-2-methylpiperazin-1- | | |
| | yl)pyrimidine-2-carbonitrile | | |
| 207 | (S)-2-(4-(4-(2-(2-aminopyridin-3-yl)-5-phenyl-3H- | 579.4 | ++ |
| | imidazo[4,5-b]pyridin-3-yl)benzyl)-2-methylpiperazin-1- | | |
| | yl)pyrimidine-4-carbonitrile | | |
| 208 | (S)-6-(4-(4-(2-(2-aminopyridin-3-yl)-5-phenyl-3H- | 579.4 | ++ |
| | imidazo[4,5-b]pyridin-3-yl)benzyl)-2-methylpiperazin-1- | | |
| | yl)pyrimidine-4-carbonitrile | | |
| 209 | 4-((4-(2-(2-aminopyridin-3-yl)-5-phenyl-3H-imidazo[4,5- | 497.2 | ++++ |
| | b]pyridin-3-yl)benzyl)amino)-1,3,5-triazine-2-carbonitrile | | |
| 210 | 4-((4-(2-(2-aminopyridin-3-yl)-5-phenyl-3H-imidazo[4,5- | 513.34 | + |
| | b]pyridin-3-yl)benzyl)amino)-6-hydroxy-1,3,5-triazine-2- | | |
| | carbonitrile | | |
| 211 | 4-(8-(4-(2-(2-aminopyridin-3-yl)-5-phenyl-3H-imidazo[4,5- | 591.4 | ++++ |
| | b]pyridin-3-yl)benzyl)-3,8-diazabicyclo[3.2.1]octan-3- | | |
| | yl)pyrimidine-2-carbonitrile | | |
| 212 | 2-(8-(4-(2-(2-aminopyridin-3-yl)-5-phenyl-3H-imidazo[4,5- | 591.3 | ++ |
| | b]pyridin-3-yl)benzyl)-3,8-diazabicyclo[3.2.1]octan-3- | | |
| | yl)pyrimidine-4-carbonitrile | | |
| 213 | 6-(8-(4-(2-(2-aminopyridin-3-yl)-5-phenyl-3H-imidazo[4,5- | 591.3 | +++ |
| | b]pyridin-3-yl)benzyl)-3,8-diazabicyclo[3.2.1]octan-3- | | |
| | yl)pyrimidine-4-carbonitrile | | |
| 214 | 4-(4-(4-(2-(2-aminopyridin-3-yl)-5-phenyl-3H-imidazo[4,5- | 579.2 | +++ |
| | b]pyridin-3-yl)benzyl)-1,4-diazepan-1-yl)pyrimidine-2- | | |
| | carbonitrile | | |
| 215 | 6-(4-(4-(2-(2-aminopyridin-3-yl)-5-phenyl-3H-imidazo[4,5- | 579.3 | ++++ |
| | b]pyridin-3-yl)benzyl)-1,4-diazepan-1-yl)pyrimidine-4- | | |
| | carbonitrile | | |
| 216 | 4-((1R,4R)-5-(4-(2-(2-aminopyridin-3-yl)-5-phenyl-3H- | 577.3 | ++++ |
| | imidazo[4,5-b]pyridin-3-yl)benzyl)-2,5- | | |
| | diazabicyclo[2.2.1]heptan-2-yl)pyrimidine-2-carbonitrile | | |
| 217 | 2-((1R,4R)-5-(4-(2-(2-aminopyridin-3-yl)-5-phenyl-3H- | 577.4 | +++ |
| | imidazo[4,5-b]pyridin-3-yl)benzyl)-2,5- | | |
| | diazabicyclo[2.2.1]heptan-2-yl)pyrimidine-4-carbonitrile | | |
| 218 | 6-((IR,4R)-5-(4-(2-(2-aminopyridin-3-yl)-5-phenyl-3H- | 577.4 | +++ |
| | imidazo[4,5-b]pyridin-3-yl)benzyl)-2,5- | | |
| | diazabicyclo[2.2.1]heptan-2-yl)pyrimidine-4-carbonitrile | | |
| 219 | 4-(5-(4-(2-(2-aminopyridin-3-yl)-5-phenyl-3H-imidazo[4,5- | 591.3 | ++++ |
| | b]pyridin-3-yl)benzyl)-2,5-diazabicyclo[2.2.2]octan-2- | | |
| | yl)pyrimidine-2-carbonitrile | | |
| 220 | 2-(5-(4-(2-(2-aminopyridin-3-yl)-5-phenyl-3H-imidazo[4,5- | 591.4 | ++ |
| | b]pyridin-3-yl)benzyl)-2,5-diazabicyclo[2.2.2]octan-2- | | |
| | yl)pyrimidine-4-carbonitrile | | |
| 221 | 6-(5-(4-(2-(2-aminopyridin-3-yl)-5-phenyl-3H-imidazo[4,5- | 591.3 | ++ |
| | b]pyridin-3-yl)benzyl)-2,5-diazabicyclo[2.2.2]octan-2- | | |
| | yl)pyrimidine-4-carbonitrile | | |
| 222 | 4-((1-((4-(2-(2-aminopyridin-3-yl)-5-phenyl-3H-imidazo[4,5- | 581.3 | ++++ |
| | b]pyridin-3-yl)phenyl)methyl-d2)piperidin-4- | | |
| | ylamino)pyrimidine-2-carbonitrile | | |
| 223 | 4-((1-(4-(2-(2-aminopyridin-3-yl)-5-phenyl-3H-imidazo[4,5- | 594.2 | +++ |
| | b]pyridin-3-yl)benzyl)piperidin-4-yl)(methyl)amino)-1,3,5- | | |
| | triazine-2-carbonitrile | | |
| 224 | (S)-4-((1-(4-(2-(2-aminopyridin-3-yl)-5-phenyl-3H- | 580.3 | ++++ |
| | imidazo[4,5-b]pyridin-3-yl)benzyl)piperidin-3-yl)amino)- | | |
| | 1,3,5-triazine-2-carbonitrile | | |
| 225 | (R)-4-(4-(4-(2-(2-aminopyridin-3-yl)-5-phenyl-3H- | 579.3 | ++ |
| | imidazo[4,5-b]pyridin-3-yl)benzyl)-2-methylpiperazin-1- | | |
| | yl)pyrimidine-2-carbonitrile | | |
| 226 | (R)-2-(4-(4-(2-(2-aminopyridin-3-yl)-5-phenyl-3H- | 579.3 | ++ |
| | imidazo[4,5-b]pyridin-3-yl)benzyl)-2-methylpiperazin-1- | | |
| | yl)pyrimidine-4-carbonitrile | | |
| 227 | (R)-6-(4-(4-(2-(2-aminopyridin-3-yl)-5-phenyl-3H- | 579.3 | ++ |
| | imidazo[4,5-b]pyridin-3-yl)benzyl)-2-methylpiperazin-1- | | |
| | yl)pyrimidine-4-carbonitrile | | |
| 228 | 2-(4-(4-(2-(2-aminopyridin-3-yl)-5-phenyl-3H-imidazo[4,5- | 579.3 | +++ |
| | b]pyridin-3-yl)benzyl)-1,4-diazepan-1-yl)pyrimidine-4- | | |
| | carbonitrile | | |
| 229 | N-(3-(4-(2-(2-aminopyridin-3-yl)-5-phenyl-3H-imidazo[4,5- | 633.3 | +++ |
| | b]pyridin-3-yl)benzyl)-3-azabicyclo[3.2.1]octan-8-yl)-2- | | |
| | cyanopyrimidine-4-carboxamide | | |
| 230 | N-(3-(4-(2-(2-aminopyridin-3-yl)-5-phenyl-3H-imidazo[4,5- | 633.3 | ++ |
| | b]pyridin-3-yl)benzyl)-3-azabicyclo[3.2.1]octan-8-yl)-6- | | |
| | cyanopyrimidine-4-carboxamide | | |
| 231 | 2-((3-(4-(2-(2-aminopyridin-3-yl)-5-phenyl-3H-imidazo[4,5- | 605.3 | + |
| | b]pyridin-3-yl)benzyl)-3-azabicyclo[3.2.1]octan-8- | | |
| | yl)amino)pyrimidine-4-carbonitrile | | |
| 232 | 4-((1-(4-(2-(2-aminopyridin-3-yl)-5-phenyl-3H-imidazo[4,5- | 596.2 | ++++ |
| | b]pyridin-3-yl)benzyl)piperidin-4-yl)(methyl- | | |
| | d3)amino)pyrimidine-2-carbonitrile | | |
| 233 | 4-((1-(4-(2-(2-aminopyridin-3-yl)-5-phenyl-3H-imidazo[4,5- | 597.4 | +++ |
| | b]pyridin-3-yl)benzyl)piperidin-4-yl)(methyl-d3)amino)-1,3,5- | | |
| | triazine-2-carbonitrile | | |
| 234 | N-(1-(4-(2-(2-aminopyridin-3-yl)-5-phenyl-3H-imidazo[4,5- | 624.4 | ++++ |
| | b]pyridin-3-yl)benzyl)piperidin-4-yl)-2-cyano-N-(methyl- | | |
| | d3)pyrimidine-4-carboxamide | | |
| 235 | 4-(4-(4-(2-(2-aminopyridin-3-yl)-5-morpholino-3H- | 574.3 | ++ |
| | imidazo[4,5-b]pyridin-3-yl)benzyl)piperazin-1-yl)pyrimidine- | | |
| | 2-carbonitrile | | |
| 236 | 2-(4-(4-(2-(2-aminopyridin-3-yl)-5-morpholino-3H- | 574.3 | +++ |
| | imidazo[4,5-b]pyridin-3-yl)benzyl)piperazin-1-yl)pyrimidine- | | |
| | 4-carbonitrile | | |
| 237 | 6-(4-(4-(2-(2-aminopyridin-3-yl)-5-morpholino-3H- | 574.2 | +++ |
| | imidazo[4,5-b]pyridin-3-yl)benzyl)piperazin-1-yl)pyrimidine- | | |
| | 4-carbonitrile | | |
| 238 | 4-(4-(4-(2-(2-aminopyridin-3-yl)-5-(6-oxo-1,6-dihydropyridin- | 582.2 | +++ |
| | 2-yl)-3H-imidazo[4,5-b]pyridin-3-yl)benzyl)piperazin-1- | | |
| | yl)pyrimidine-2-carbonitrile | | |
| 239 | 4-((1-(4-(2-(2-aminopyridin-3-yl)-5-morpholino-3H- | 588.4 | ++++ |
| | imidazo[4,5-b]pyridin-3-yl)benzyl)piperidin-4- | | |
| | yl)amino)pyrimidine-2-carbonitrile | | |
| 240 | 2-((1-(4-(2-(2-aminopyridin-3-yl)-5-morpholino-3H- | 588.3 | +++ |
| | imidazo[4,5-b]pyridin-3-yl)benzyl)piperidin-4- | | |
| | yl)amino)pyrimidine-4-carbonitrile | | |
| 241 | 6-((1-(4-(2-(2-aminopyridin-3-yl)-5-morpholino-3H- | 588.3 | +++ |
| | imidazo[4,5-b]pyridin-3-yl)benzyl)piperidin-4- | | |
| | yl)amino)pyrimidine-4-carbonitrile | | |
| 242 | 4-(4-(4-(2-(2-aminopyridin-3-yl)-5-(4-fluorophenyl)-3H- | 583.3 | +++ |
| | imidazo[4,5-b]pyridin-3-yl)benzyl)piperazin-1-yl)pyrimidine- | | |
| | 2-carbonitrile | | |
| 243 | 2-(4-(4-(2-(2-aminopyridin-3-yl)-5-(4-fluorophenyl)-3H- | 583.3 | ++ |
| | imidazo[4,5-b]pyridin-3-yl)benzyl)piperazin-1-yl)pyrimidine- | | |
| | 4-carbonitrile | | |
| 244 | N-(4-(2-(2-aminopyridin-3-yl)-3H-imidazo[4,5-b]pyridin-3- | 453.0 | + |
| | yl)benzyl)-2-cyanothiazole-4-carboxamide | | |
| 245 | N-(4-(2-(2-aminopyridin-3-yl)-5-phenyl-3H-imidazo[4,5- | 529.2 | ++++ |
| | b]pyridin-3-yl)benzyl)-2-cyanothiazole-5-carboxamide | | |
| 246 | N-(4-(2-(2-aminopyridin-3-yl)-5-phenyl-3H-imidazo[4,5- | 529.2 | +++ |
| | b]pyridin-3-yl)benzyl)-2-cyanothiazole-4-carboxamide | | |
| 247 | 4-(4-(4-(2-(2-aminopyridin-3-yl)-5-phenyl-3H-imidazo[4,5- | 598.2 | ++++ |
| | b]pyridin-3-yl)benzyl)piperazine-1-carbonyl)thiazole-2- | | |
| | carbonitrile | | |
| 248 | 4-((7-(4-(2-(2-aminopyridin-3-yl)-5-phenyl-3H-imidazo[4,5- | 620.4 | ++++ |
| | b]pyridin-3-yl)benzyl)-7-azaspiro[3.5]nonan-2-yl)amino)- | | |
| | 1,3,5-triazine-2-carbonitrile | | |
| 249 | 4-(7-(4-(2-(2-aminopyridin-3-yl)-5-phenyl-3H-imidazo[4,5- | 606.4 | +++ |
| | b]pyridin-3-yl)benzyl)-2,7-diazaspiro[3.5]nonan-2-yl)-1,3,5- | | |
| | triazine-2-carbonitrile | | |
| 250 | 4-(4-(4-(2-(2-aminopyridin-3-yl)-5-phenyl-3H-imidazo[4,5- | 580.3 | +++ |
| | b]pyridin-3-yl)benzyl)-1,4-diazepan-1-yl)-1,3,5-triazine-2- | | |
| | carbonitrile | | |
| 251 | 4-(7-(4-(2-(2-aminopyridin-3-yl)-5-phenyl-3H-imidazo[4,5- | 606.4 | ++++ |
| | b]pyridin-3-yl)benzyl)-2,7-diazaspiro[4.4]nonan-2-y])-1,3,5- | | |
| | triazine-2-carbonitrile | | |
| 252 | 4-(2-(4-(2-(2-aminopyridin-3-yl)-5-phenyl-3H-imidazo[4,5- | 620.3 | +++ |
| | b]pyridin-3-yl)benzyl)-2,8-diazaspiro[4.5]decan-8-yl)-1,3,5- | | |
| | triazine-2-carbonitrile | | |
| 253 | 4-(2-(4-(2-(2-aminopyridin-3-yl)-5-phenyl-3H-imidazo[4,5- | 620.5 | +++ |
| | b]pyridin-3-yl)benzyl)-2,7-diazaspiro[4.5]decan-7-yl)-1,3,5- | | |
| | triazine-2-carbonitrile | | |
| 254 | 4-(9-(4-(2-(2-aminopyridin-3-yl)-5-phenyl-3H-imidazo[4,5- | 634.3 | ++++ |
| | b]pyridin-3-yl)benzyl)-2,9-diazaspiro[5.5]undecan-2-yl)-1,3,5- | | |
| | triazine-2-carbonitrile | | |
| 255 | (R)-4-(7-(4-(2-(2-aminopyridin-3-yl)-5-phenyl-3H- | 606.3 | ++++ |
| | imidazo[4,5-b]pyridin-3-yl)benzyl)-2,7-diazaspiro[4.4]nonan- | | |
| | 2-yl)-1,3,5-triazine-2-carbonitrile | | |
| 256 | 4-((2R,6R)-4-(4-(2-(2-aminopyridin-3-yl)-5-phenyl-3H- | 594.3 | +++ |
| | imidazo[4,5-b]pyridin-3-yl)benzyl)-2,6-dimethylpiperazin-1- | | |
| | yl)-1,3,5-triazine-2-carbonitrile | | |
| 257 | 4-(5-(4-(2-(2-aminopyridin-3-yl)-5-phenyl-3H-imidazo[4,5- | 592.2 | +++ |
| | b]pyridin-3-yl)benzyl)-2,5-diazabicyclo[2.2.2]octan-2-yI)- | | |
| | 1,3,5-triazine-2-carbonitrile | | |
| 258 | 4-(4-(4-(2-(2-aminopyridin-3-yl)-5-cyclopropyl-3H- | 529.3 | ++++ |
| | imidazo[4,5-b]pyridin-3-yl)benzyl)piperazin-1-yl)pyrimidine- | | |
| | 2-carbonitrile | | |
| 259 | 4-(4-(4-(2-(2-aminopyridin-3-yl)-5-(2-oxo-1,2-dihydropyridin- | 582.3 | ++++ |
| | 3-yl)-3H-imidazo[4,5-b]pyridin-3-yl)benzyl)piperazin-1- | | |
| | yl)pyrimidine-2-carbonitrile | | |
| 260 | 4-(4-(4-(2-(2-aminopyridin-3-yl)-5-(1-methyl-2-oxo-1,2- | 596.3 | +++ |
| | dihydropyridin-3-yl)-3H-imidazo[4,5-b]pyridin-3- | | |
| | yl)benzyl)piperazin-1-yl)pyrimidine-2-carbonitrile | | |
| 261 | 4-((1-(4-(2-(2-aminopyridin-3-yl)-5-(1-methyl-2-oxo-1,2- | 610.4 | ++++ |
| | dihydropyridin-3-yl)-3H-imidazo[4,5-b]pyridin-3- | | |
| | yl)benzyl)piperidin-4-yl)amino)pyrimidine-2-carbonitrile | | |
| 262 | 4-((1-(4-(2-(2-aminopyridin-3-yl)-5-cyclobutyl-3H- | 557.4 | ++++ |
| | imidazo[4,5-b]pyridin-3-yl)benzyl)piperidin-4- | | |
| | yl)amino)pyrimidine-2-carbonitrile | | |
| 263 | 4-((1-(4-(2-(2-aminopyridin-3-yl)-5-(cyclohex-1-en-1-yl)-3H- | 583.4 | +++ |
| | imidazo[4,5-b]pyridin-3-yl)benzyl)piperidin-4- | | |
| | yl)amino)pyrimidine-2-carbonitrile | | |
| 264 | 4-((1-(4-(2-(2-aminopyridin-3-yl)-5-cyclohexyl-3H- | 585.4 | ++++ |
| | imidazo[4,5-b]pyridin-3-yl)benzyl)piperidin-4- | | |
| | yl)amino)pyrimidine-2-carbonitrile | | |
| 265 | 4-((1-(4-(2-(2-aminopyridin-3-yl)-5-(3-cyanophenyl)-3H- | 604.4 | ++++ |
| | imidazo[4,5-b]pyridin-3-yl)benzyl)piperidin-4- | | |
| | yl)amino)pyrimidine-2-carbonitrile | | |
| 266 | 4-((1-(4-(2-(2-aminopyridin-3-yl)-5-(6-oxo-1,6- | 596.4 | ++ |
| | dihydropyridin-3-yl)-3H-imidazo[4,5-b]pyridin-3- | | |
| | yl)benzyl)piperidin-4-yl)amino)pyrimidine-2-carbonitrile | | |
| 267 | 4-((1-(4-(2-(2-aminopyridin-3-yl)-5-(6-oxo-1,6- | 596.1 | ++++ |
| | dihydropyridin-2-yl)-3H-imidazo[4,5-b]pyridin-3- | | |
| | yl)benzyl)piperidin-4-yl)amino)pyrimidine-2-carbonitrile | | |
| 268 | 4-(2-(4-(2-(2-aminopyridin-3-yl)-5-phenyl-3H-imidazo[4,5- | 606.3 | ++++ |
| | bjpyridin-3-yl)benzyl)-2,7-diazaspiro[3.5]nonan-7-yl)-1,3,5- | | |
| | triazine-2-carbonitrile | | |
| 269 | (S)-4-(7-(4-(2-(2-aminopyridin-3-yl)-5-phenyl-3H- | 606.4 | ++++ |
| | imidazo[4,5-b]pyridin-3-yl)benzyl)-2,7-diazaspiro[4.4]nonan- | | |
| | 2-yl)-1,3,5-triazine-2-carbonitrile | | |
| 270 | 4-(3-(4-(2-(2-aminopyridin-3-yl)-5-phenyl-3H-imidazo[4,5- | 592.4 | ++ |
| | b]pyridin-3-yl)benzyl)-3,8-diazabicyclo[3.2.1]octan-8-yl)- | | |
| | 1,3,5-triazine-2-carbonitrile | | |
| 271 | 4-(8-(4-(2-(2-aminopyridin-3-yl)-5-phenyl-3H-imidazo[4,5- | 592.4 | +++ |
| | b]pyridin-3-yl)benzyl)-3,8-diazabicyclo[3.2.1]octan-3-yl)- | | |
| | 1,3,5-triazine-2-carbonitrile | | |
| 272 | 4-((1R,4R)-5-(4-(2-(2-aminopyridin-3-yl)-5-phenyl-3H- | 578.3 | ++++ |
| | imidazo[4,5-b]pyridin-3-yl)benzyl)-2,5- | | |
| | diazabicyclo[2.2.1]heptan-2-yl)-1,3,5-triazine-2-carbonitrile | | |
| 273 | (R)-4-(4-(4-(2-(2-aminopyridin-3-yl)-5-phenyl-3H- | 580.3 | ++++ |
| | imidazo[4,5-b]pyridin-3-yl)benzyl)-2-methylpiperazin-1-yl)- | | |
| | 1,3,5-triazine-2-carbonitrile | | |
| 274 | (S)-4-(4-(4-(2-(2-aminopyridin-3-yl)-5-phenyl-3H- | 580.4 | +++ |
| | imidazo[4,5-b]pyridin-3-yl)benzyl)-2-methylpiperazin-1-yl)- | | |
| | 1,3,5-triazine-2-carbonitrile | | |
| 275 | 4-((2S,6S)-4-(4-(2-(2-aminopyridin-3-yl)-5-phenyl-3H- | 594.4 | ++++ |
| | imidazo[4,5-b]pyridin-3-yl)benzyl)-2,6-dimethylpiperazin-1- | | |
| | yl)-1,3,5-triazine-2-carbonitrile | | |
| 276 | 4-(7-(4-(2-(2-aminopyridin-3-yl)-5-phenyl-3H-imidazo[4,5- | 592.3 | ++++ |
| | b]pyridin-3-yl)benzyl)-4,7-diazaspiro[2.5]octan-4-yl)-1,3,5- | | |
| | triazine-2-carbonitrile | | |
| 277 | 4-((2R,6S)-4-(4-(2-(2-aminopyridin-3-yl)-5-phenyl-3H- | 594.4 | +++ |
| | imidazo[4,5-b]pyridin-3-yl)benzyl)-2,6-dimethylpiperazin-1- | | |
| | yl)-1,3,5-triazine-2-carbonitrile | | |
| 278 | 4-((1-(4-(2-(2-aminopyridin-3-yl)-5-phenyl-3H-imidazo[4,5- | 594.4 | +++ |
| | b]pyridin-3-yl)benzyl)azepan-4-yl)amino)-1,3,5-triazine-2- | | |
| | carbonitrile | | |
| 279 | 4-((4-(4-(2-(2-aminopyridin-3-yl)-5-phenyl-3H-imidazo[4,5- | 596.4 | ++++ |
| | b]pyridin-3-yl)benzyl)-1,4-oxazepan-6-yl)amino)-1,3,5- | | |
| | triazine-2-carbonitrile | | |
| 280 | 4-((1-(4-(2-(2-aminopyridin-3-yl)-5-methyl-3H-imidazo[4,5- | 517.2 | ++++ |
| | b]pyridin-3-yl)benzyl)piperidin-4-yl)amino)pyrimidine-2- | | |
| | carbonitrile | | |
| 281 | 4-(4-(4-(2-(2-aminopyridin-3-yl)-5-(2-fluorophenyl)-3H- | 583.3 | ++++ |
| | imidazo[4,5-b]pyridin-3-yl)benzyl)piperazin-1-yl)pyrimidine- | | |
| | 2-carbonitrile | | |
| 282 | 2-(4-(4-(2-(2-aminopyridin-3-yl)-5-(2-fluorophenyl)-3H- | 583.3 | +++ |
| | imidazo[4,5-b]pyridin-3-yl)benzyl)piperazin-1-yl)pyrimidine- | | |
| | 4-carbonitrile | | |
| 283 | 2-(4-(4-(2-(2-aminopyridin-3-yl)-5-(3-oxo-1,4-diazepan-1-y])- | 601.4 | +++ |
| | 3H-imidazo[4,5-b]pyridin-3-yl)benzyl)piperazin-1- | | |
| | yl)pyrimidine-4-carbonitrile | | |
| 284 | 6-(4-(4-(2-(2-aminopyridin-3-yl)-5-(3-oxo-1,4-diazepan-1-yl)- | 601.4 | ++ |
| | 3H-imidazo[4,5-b]pyridin-3-yl)benzyl)piperazin-1- | | |
| | yl)pyrimidine-4-carbonitrile | | |
| 285 | 4-(8-(4-(2-(2-aminopyridin-3-yl)-5-morpholino-3H- | 601.5 | ++++ |
| | imidazo[4,5-b]pyridin-3-yl)benzyl)-3,8- | | |
| | diazabicyclo[3.2.1]octan-3-yl)-1,3,5-triazine-2-carbonitrile | | |
| 286 | 4-((1-(4-(2-(2-aminopyridin-3-yl)-5-isopropyl-3H- | 545.2 | ++++ |
| | imidazo[4,5-b]pyridin-3-yl)benzyl)piperidin-4- | | |
| | yl)amino)pyrimidine-2-carbonitrile | | |
| 287 | 4-((1-(4-(2-(2-aminopyridin-3-yl)-5-methoxy-3H-imidazo[4,5- | 533.2 | ++++ |
| | b]pyridin-3-yl)benzyl)piperidin-4-yl)amino)pyrimidine-2- | | |
| | carbonitrile | | |
| 288 | 2-(2-aminopyridin-3-yl)-3-(4-((4-((2-cyanopyrimidin-4- | 546.2 | +++ |
| | yl)amino)piperidin-1-yl)methyl)phenyl)-3H-imidazo[4,5- | | |
| | b]pyridine-5-carboxamide | | |
| 289 | 2-(2-aminopyridin-3-yl)-3-(4-((4-((2-cyanopyrimidin-4- | 574.4 | ++ |
| | yl)amino)piperidin-1-yl)methyl)phenyl)-N,N-dimethyl-3H- | | |
| | imidazo[4,5-b]pyridine-5-carboxamide | | |
| 290 | 6-(4-(4-(2-(2-aminopyridin-3-yl)-5-cyclopropyl-3H- | 529.4 | +++ |
| | imidazo[4,5-b]pyridin-3-yl)benzyl)piperazin-1-yl)pyrimidine- | | |
| | 4-carbonitrile | | |
| 291 | 6-((1-(4-(2-(2-aminopyridin-3-yl)-5-cyclopropyl-3H- | 543.4 | +++ |
| | imidazo[4,5-b]pyridin-3-yl)benzyl)piperidin-4- | | |
| | yl)amino)pyrimidine-4-carbonitrile | | |
| 292 | 4-(4-(4-(2-(2-aminopyridin-3-yl)-5-morpholino-3H- | 575.4 | ++++ |
| | imidazo[4,5-b]pyridin-3-yl)benzyl)piperazin-1-yl)-1,3,5- | | |
| | triazine-2-carbonitrile | | |
| 293 | 4-(4-(4-(2-(2-aminopyridin-3-yl)-5-(2-cyanophenyl)-3H- | 590.2 | ++++ |
| | imidazo[4,5-b]pyridin-3-yl)benzyl)piperazin-1-yl)pyrimidine- | | |
| | 2-carbonitrile | | |
| 294 | 4-(4-(4-(2-(2-aminopyridin-3-yl)-5-(4-cyanophenyl)-3H- | 590.3 | +++ |
| | imidazo[4,5-b]pyridin-3-yl)benzyl)piperazin-1-yl)pyrimidine- | | |
| | 2-carbonitrile | | |
| 295 | 4-(4-(4-(2-(2-aminopyridin-3-yl)-5-(3-fluorophenyl)-3H- | 583.3 | ++++ |
| | imidazo[4,5-b]pyridin-3-yl)benzyl)piperazin-1-yl)pyrimidine- | | |
| | 2-carbonitrile | | |
| 296 | 4-(4-(4-(2-(2-aminopyridin-3-yl)-5-(3-chlorophenyl)-3H- | 599.2 | ++++ |
| | imidazo[4,5-b]pyridin-3-yl)benzyl)piperazin-1-yl)pyrimidine- | | |
| | 2-carbonitrile | | |
| 297 | 2-(4-(4-(2-(2-aminopyridin-3-yl)-5-(3-chlorophenyl)-3H- | 599.4 | ++ |
| | imidazo[4,5-b]pyridin-3-yl)benzyl)piperazin-1-yl)pyrimidine- | | |
| | 4-carbonitrile | | |
| 298 | 4-((1-(4-(2-(2-aminopyridin-3-yl)-5-cyclopropyl-3H- | 543.3 | ++++ |
| | imidazo[4,5-b]pyridin-3-yl)benzyl)piperidin-4- | | |
| | yl)amino)pyrimidine-2-carbonitrile | | |
| 299 | 4-((1-(4-(2-(2-aminopyridin-3-yl)-5-(tetrahydro-2H-pyran-4- | 587.2 | ++++ |
| | yl)-3H-imidazo[4,5-b]pyridin-3-yl)benzyl)piperidin-4- | | |
| | yl)amino)pyrimidine-2-carbonitrile | | |
| 300 | 4-((1-(4-(2-(2-aminopyridin-3-yl)-5-phenyl-3H-imidazo[4,5- | 552.4 | +++ |
| | b]pyridin-3-yl)benzyl)azetidin-3-yl)amino)-1,3,5-triazine-2- | | |
| | carbonitrile | | |
| 301 | 4-(6-(4-(2-(2-aminopyridin-3-yl)-5-phenyl-3H-imidazo[4,5- | 592.3 | ++++ |
| | b]pyridin-3-yl)benzyl)-2,6-diazaspiro[3.4]octan-2-yl)-1,3,5- | | |
| | triazine-2-carbonitrile | | |
| 302 | 4-(7-(4-(2-(2-aminopyridin-3-yl)-5-phenyl-3H-imidazo[4,5- | 605.3 | ++++ |
| | b]pyridin-3-yl)benzyl)-2,7-diazaspiro[4.4]nonan-2- | | |
| | yl)pyrimidine-2-carbonitrile | | |
| 303 | 4-(6-(4-(2-(2-aminopyridin-3-yl)-5-phenyl-3H-imidazo[4,5- | 578.3 | ++++ |
| | b]pyridin-3-yl)benzyl)-2,6-diazaspiro[3.3]heptan-2-yl)-1,3,5- | | |
| | triazine-2-carbonitrile | | |
| 304 | 4-(4-(4-(2-(2-aminopyridin-3-yl)-5-(2-chlorophenyl)-3H- | 600.4 | ++++ |
| | imidazo[4,5-b]pyridin-3-yl)benzyl)piperazin-1-yl)-1,3,5- | | |
| | triazine-2-carbonitrile | | |
| 305 | 4-((1-(4-(2-(2-aminopyridin-3-yl)-5-(2-chlorophenyl)-3H- | 613.4 | ++++ |
| | imidazo[4,5-b]pyridin-3-yl)benzyl)piperidin-4- | | |
| | yl)amino)pyrimidine-2-carbonitrile | | |
| 306 | 2-((1-(4-(2-(2-aminopyridin-3-yl)-5-(2-chlorophenyl)-3H- | 613.3 | +++ |
| | imidazo[4,5-b]pyridin-3-yl)benzyl)piperidin-4- | | |
| | yl)amino)pyrimidine-4-carbonitrile | | |
| 307 | 6-((1-(4-(2-(2-aminopyridin-3-yl)-5-(2-chlorophenyl)-3H- | 613.4 | ++ |
| | imidazo[4,5-b]pyridin-3-yl)benzyl)piperidin-4- | | |
| | yl)amino)pyrimidine-4-carbonitrile | | |
| 308 | 4-((1-(4-(2-(2-aminopyridin-3-yl)-5-(4-chlorophenyl)-3H- | 613.2 | ++++ |
| | imidazo[4,5-b]pyridin-3-yl)benzyl)piperidin-4- | | |
| | yl)amino)pyrimidine-2-carbonitrile | | |
| 309 | 2-(4-(4-(2-(2-aminopyridin-3-yl)-5-(3-fluorophenyl)-3H- | 583.4 | +++ |
| | imidazo[4,5-b]pyridin-3-yl)benzyl)piperazin-1-yl)pyrimidine- | | |
| | 4-carbonitrile | | |
| 310 | 6-(4-(4-(2-(2-aminopyridin-3-yl)-5-(3-fluorophenyl)-3H- | 583.4 | ++++ |
| | imidazo[4,5-b]pyridin-3-yl)benzyl)piperazin-1-yl)pyrimidine- | | |
| | 4-carbonitrile | | |
| 311 | 6-((1-(4-(2-(2-aminopyridin-3-yl)-5-(3-fluorophenyl)-3H- | 597.4 | ++++ |
| | imidazo[4,5-b]pyridin-3-yl)benzyl)piperidin-4- | | |
| | yl)amino)pyrimidine-4-carbonitrile | | |
| 312 | 4-(4-(4-(2-(2-aminopyridin-3-yl)-5-(2-fluorophenyl)-3H- | 584.2 | ++++ |
| | imidazo[4,5-b]pyridin-3-yl)benzyl)piperazin-1-yl)-1,3,5- | | |
| | triazine-2-carbonitrile | | |
| 313 | 2-((1-(4-(2-(2-aminopyridin-3-yl)-5-(2-fluorophenyl)-3H- | 597.4 | ++++ |
| | imidazo[4,5-b]pyridin-3-yl)benzyl)piperidin-4- | | |
| | yl)amino)pyrimidine-4-carbonitrile | | |
| 314 | 6-(4-(4-(2-(2-aminopyridin-3-yl)-5-(2-fluorophenyl)-3H- | 583.4 | ++++ |
| | imidazo[4,5-b]pyridin-3-yl)benzyl)piperazin-1-yl)pyrimidine- | | |
| | 4-carbonitrile | | |
| 315 | 6-((1-(4-(2-(2-aminopyridin-3-yl)-5-(2-fluorophenyl)-3H- | 597.4 | +++ |
| | imidazo[4,5-b]pyridin-3-yl)benzyl)piperidin-4- | | |
| | yl)amino)pyrimidine-4-carbonitrile | | |
| 316 | 4-((1-(4-(2-(2-aminopyridin-3-yl)-5-(2-fluorophenyl)-3H- | 597.4 | ++++ |
| | imidazo[4,5-b]pyridin-3-yl)benzyl)piperidin-4- | | |
| | yl)amino)pyrimidine-2-carbonitrile | | |
| 317 | 4-(4-(4-(2-(2-aminopyridin-3-yl)-5-(3-oxo-1,4-diazepan-1-yl)- | 601.2 | ++ |
| | 3H-imidazo[4,5-b]pyridin-3-yl)benzyl)piperazin-1- | | |
| | yl)pyrimidine-2-carbonitrile | | |
| 318 | 4-((1-(4-(2-(2-aminopyridin-3-yl)-5-(3-oxo-1,4-diazepan-1-yl)- | 615.2 | +++ |
| | 3H-imidazo[4,5-b]pyridin-3-yl)benzyl)piperidin-4- | | |
| | yl)amino)pyrimidine-2-carbonitrile | | |
| 319 | 2-(4-(4-(2-(2-aminopyridin-3-yl)-5-(4-cyanophenyl)-3H- | 590.2 | ++ |
| | imidazo[4,5-b]pyridin-3-yl)benzyl)piperazin-1-yl)pyrimidine- | | |
| | 4-carbonitrile | | |
| 320 | 4-((1-(4-(2-(2-aminopyridin-3-yl)-5-(4-cyanophenyl)-3H- | 604.2 | ++++ |
| | imidazo[4,5-b]pyridin-3-yl)benzyl)piperidin-4- | | |
| | yl)amino)pyrimidine-2-carbonitrile | | |
| 321 | 2-((1-(4-(2-(2-aminopyridin-3-yl)-5-(4-cyanophenyl)-3H- | 604.4 | ++ |
| | imidazo[4,5-b]pyridin-3-yl)benzyl)piperidin-4- | | |
| | yl)amino)pyrimidine-4-carbonitrile | | |
| 322 | 2-(4-(4-(2-(2-aminopyridin-3-yl)-5-(2-cyanophenyl)-3H- | 590.2 | +++ |
| | imidazo[4,5-b]pyridin-3-yl)benzyl)piperazin-1-yl)pyrimidine- | | |
| | 4-carbonitrile | | |
| 323 | 4-((1-(4-(2-(2-aminopyridin-3-yl)-5-(5-oxo-1,4-diazepan-1-yl)- | 615.5 | + |
| | 3H-imidazo[4,5-b]pyridin-3-yl)benzyl)piperidin-4- | | |
| | yl)amino)pyrimidine-2-carbonitrile | | |
| 324 | 4-(4-(4-(2-(2-aminopyridin-3-yl)-5-(2-chlorophenyl)-3H- | 599.1 | ++++ |
| | imidazo[4,5-b]pyridin-3-yl)benzyl)piperazin-1-yl)pyrimidine- | | |
| | 2-carbonitrile | | |
| 325 | 2-(4-(4-(2-(2-aminopyridin-3-yl)-5-(2-chlorophenyl)-3H- | 599.3 | +++ |
| | imidazo[4,5-b]pyridin-3-yl)benzyl)piperazin-1-yl)pyrimidine- | | |
| | 4-carbonitrile | | |
| 326 | 6-(4-(4-(2-(2-aminopyridin-3-yl)-5-(2-chlorophenyl)-3H- | 599.4 | +++ |
| | imidazo[4,5-b]pyridin-3-yl)benzyl)piperazin-1-yl)pyrimidine- | | |
| | 4-carbonitrile | | |
| 327 | 5-(4-(4-(2-(2-aminopyridin-3-yl)-5-phenyl-3H-imidazo[4,5- | 598.3 | ++ |
| | b]pyridin-3-yl)benzyl)piperazine-1-carbonyl)thiazole-2- | | |
| | carbonitrile | | |
| 328 | 4-(4-(4-(2-(2-aminopyridin-3-yl)-5-phenyl-3H-imidazo[4,5- | 566.3 | ++++ |
| | b]pyridin-3-yl)benzyl)piperazin-1-yl)-1,3,5-triazine-2- | | |
| | carbonitrile | | |
| 329 | 4-(4-(4-(2-(2-aminopyridin-3-yl)-5-(cyclohex-1-en-1-yl)-3H- | 569.2 | +++ |
| | imidazo[4,5-b]pyridin-3-yl)benzyl)piperazin-1-yl)pyrimidine- | | |
| | 2-carbonitrile | | |
| 330 | 4-(4-(4-(2-(2-aminopyridin-3-yl)-5-(3,6-dihydro-2H-pyran-4- | 571.1 | ++++ |
| | yl)-3H-imidazo[4,5-b]pyridin-3-yl)benzyl)piperazin-1- | | |
| | yl)pyrimidine-2-carbonitrile | | |
| 331 | 4-(4-(4-(2-(2-aminopyridin-3-yl)-5-(3-cyanophenyl)-3H- | 590.4 | ++++ |
| | imidazo[4,5-b]pyridin-3-yl)benzyl)piperazin-1-yl)pyrimidine- | | |
| | 2-carbonitrile | | |
| 332 | 4-(4-(4-(2-(2-aminopyridin-3-yl)-5-(6-oxo-1,6-dihydropyridin- | 582.2 | + |
| | 3-yl)-3H-imidazo[4,5-b]pyridin-3-yl)benzyl)piperazin-1- | | |
| | yl)pyrimidine-2-carbonitrile | | |
| 333 | 2-(4-(4-(2-(2-aminopyridin-3-yl)-5-(6-oxo-1,6-dihydropyridin- | 582.2 | +++ |
| | 2-yl)-3H-imidazo[4,5-b]pyridin-3-yl)benzyl)piperazin-1- | | |
| | yl)pyrimidine-4-carbonitrile | | |
| 334 | 6-(4-(4-(2-(2-aminopyridin-3-yl)-5-(6-oxo-1,6-dihydropyridin- | 582.3 | +++ |
| | 2-yl)-3H-imidazo[4,5-b]pyridin-3-yl)benzyl)piperazin-1- | | |
| | yl)pyrimidine-4-carbonitrile | | |
| 335 | 4-((1-(4-(2-(2-aminopyridin-3-yl)-5-(2-cyanophenyl)-3H- | 604.3 | ++++ |
| | imidazo[4,5-b]pyridin-3-yl)benzyl)piperidin-4- | | |
| | yl)amino)pyrimidine-2-carbonitrile | | |
| 336 | N-(1-(4-(2-(2-aminopyridin-3-yl)-5-phenyl-3H-imidazo[4,5- | 621.3 | ++++ |
| | b]pyridin-3-yl)benzyl)piperidin-4-yl)-2-cyano-N- | | |
| | methylpyrimidine-4-carboxamide | | |
| 337 | (R)-4-((1-(4-(2-(2-aminopyridin-3-yl)-5-phenyl-3H- | 580.4 | ++++ |
| | imidazo[4,5-b]pyridin-3-yl)benzyl)piperidin-3-yl)amino)- | | |
| | 1,3,5-triazine-2-carbonitrile | | |
| 338 | 4-(18,4S)-5-(4-(2-(2-aminopyridin-3-yl)-5-phenyl-3H- | 578.3 | +++ |
| | imidazo[4,5-b]pyridin-3-yl)benzyl)-2,5- | | |
| | diazabicyclo[2.2.1]heptan-2-yl)-1,3,5-triazine-2-carbonitrile | | |
| 339 | 4-((3aR,6aS)-5-(4-(2-(2-aminopyridin-3-yl)-5-phenyl-3H- | 591.2 | +++ |
| | imidazo[4,5-b]pyridin-3-yl)benzyl)hexahydropyrrolo[3,4- | | |
| | c]pyrrol-2(1H)-yl)pyrimidine-2-carbonitrile | | |
| 340 | 4-((3aR,6aS)-5-(4-(2-(2-aminopyridin-3-yl)-5-phenyl-3H- | 592.4 | ++++ |
| | imidazo[4,5-b]pyridin-3-yl)benzyl)hexahydropyrrolo[3,4- | | |
| | c]pyrrol-2(1H)-yl)-1,3,5-triazine-2-carbonitrile | | |
| 341 | 4-((3aR,6aR)-5-(4-(2-(2-aminopyridin-3-yl)-5-phenyl-3H- | 591.4 | ++++ |
| | imidazo[4,5-b]pyridin-3-yl)benzyl)hexahydropyrrolo[3,4- | | |
| | clpyrrol-2(1H)-yl)pyrimidine-2-carbonitrile | | |
| 342 | 4-(4-(4-(2-(2-aminopyridin-3-yl)-5-(4-chlorophenyl)-3H- | 599.4 | +++ |
| | imidazo[4,5-b]pyridin-3-yl)benzyl)piperazin-1-yl)pyrimidine- | | |
| | 2-carbonitrile | | |
| 343 | 2-(4-(4-(2-(2-aminopyridin-3-yl)-5-(4-chlorophenyl)-3H- | 599.2 | ++ |
| | imidazo[4,5-b]pyridin-3-yl)benzyl)piperazin-1-yl)pyrimidine- | | |
| | 4-carbonitrile | | |
| 344 | 6-(4-(4-(2-(2-aminopyridin-3-yl)-5-(4-chlorophenyl)-3H- | 599.2 | ++ |
| | imidazo[4,5-b]pyridin-3-yl)benzyl)piperazin-1-yl)pyrimidine- | | |
| | 4-carbonitrile | | |
| 345 | 4-(4-(4-(2-(2-aminopyridin-3-yl)-5-(4-methyl-5-oxo-1,4- | 615.3 | ++ |
| | diazepan-1-yl)-3H-imidazo[4,5-b]pyridin-3- | | |
| | yl)benzyl)piperazin-1-yl)pyrimidine-2-carbonitrile | | |
| 346 | 4-((1-(4-(2-(2-aminopyridin-3-yl)-5-(4-methyl-5-oxo-1,4- | 629.4 | ++ |
| | diazepan-1-yl)-3H-imidazo[4,5-b]pyridin-3- | | |
| | yl)benzyl)piperidin-4-yl)amino)pyrimidine-2-carbonitrile | | |
| 347 | 2-((1-(4-(2-(2-aminopyridin-3-yl)-5-(3-oxo-1,4-diazepan-1-yl)- | 615.5 | ++ |
| | 3H-imidazo[4,5-b]pyridin-3-yl)benzyl)piperidin-4- | | |
| | yl)amino)pyrimidine-4-carbonitrile | | |
| 348 | 4-(4-(4-(2-(2-aminopyridin-3-yl)-5-(1-methyl-6-oxo-1,6- | 596.1 | ++ |
| | dihydropyridin-3-yl)-3H-imidazo[4,5-b]pyridin-3- | | |
| | yl)benzyl)piperazin-1-yl)pyrimidine-2-carbonitrile | | |
| 349 | 4-(4-(4-(2-(2-aminopyridin-3-yl)-5-(1-methyl-2-oxo-1,2- | 596.2 | +++ |
| | dihydropyridin-4-yl)-3H-imidazo[4,5-b]pyridin-3- | | |
| | yl)benzyl)piperazin-1-yl)pyrimidine-2-carbonitrile | | |
| 350 | 2-(4-(4-(2-(2-aminopyridin-3-yl)-5-cyclopropyl-3H- | 529.4 | +++ |
| | imidazo[4,5-b]pyridin-3-yl)benzyl)piperazin-1-yl)pyrimidine- | | |
| | 4-carbonitrile | | |
| 351 | 2-((1-(4-(2-(2-aminopyridin-3-yl)-5-cyclopropyl-3H- | 543.4 | +++ |
| | imidazo[4,5-b]pyridin-3-yl)benzyl)piperidin-4- | | |
| | yl)amino)pyrimidine-4-carbonitrile | | |
| 352 | 2-((1-(4-(2-(2-aminopyridin-3-yl)-5-(4-fluorophenyl)-3H- | 597.4 | +++ |
| | imidazo[4,5-b]pyridin-3-yl)benzyl)piperidin-4- | | |
| | yl)amino)pyrimidine-4-carbonitrile | | |
| 353 | 4-((1-(4-(2-(2-aminopyridin-3-yl)-5-(3-chlorophenyl)-3H- | 613.4 | ++ |
| | imidazo[4,5-b]pyridin-3-yl)benzyl)piperidin-4- | | |
| | yl)amino)pyrimidine-2-carbonitrile | | |
| 354 | 2-(4-(4-(2-(2-aminopyridin-3-yl)-5-(1-methyl-6-oxo-1,6- | 596.2 | ++ |
| | dihydropyridin-3-yl)-3H-imidazo[4,5-b]pyridin-3- | | |
| | yl)benzyl)piperazin-1-yl)pyrimidine-4-carbonitrile | | |
| 355 | 4-((1-(4-(2-(2-aminopyridin-3-yl)-5-(1-methyl-6-oxo-1,6- | 610.2 | +++ |
| | dihydropyridin-3-yl)-3H-imidazo[4,5-b]pyridin-3- | | |
| | yl)benzyl)piperidin-4-yl)amino)pyrimidine-2-carbonitrile | | |
| 356 | 2-((1-(4-(2-(2-aminopyridin-3-yl)-5-(1-methyl-6-oxo-1,6- | 610.2 | ++ |
| | dihydropyridin-3-yl)-3H-imidazo[4,5-b]pyridin-3- | | |
| | yl)benzyl)piperidin-4-yl)amino)pyrimidine-4-carbonitrile | | |
| 357 | 4-((1-(4-(2-(2-aminopyridin-3-yl)-5-(3-fluorophenyl)-3H- | 597.5 | +++ |
| | imidazo[4,5-b]pyridin-3-yl)benzyl)piperidin-4- | | |
| | yl)amino)pyrimidine-2-carbonitrile | | |
| 358 | 2-((1-(4-(2-(2-aminopyridin-3-yl)-5-(3-fluorophenyl)-3H- | 597.3 | +++ |
| | imidazo[4,5-b]pyridin-3-yl)benzyl)piperidin-4- | | |
| | yl)amino)pyrimidine-4-carbonitrile | | |
| 359 | 4-((1-(4-(2-(2-aminopyridin-3-yl)-5-(1-methyl-2-oxo-1,2- | 610.2 | ++++ |
| | dihydropyridin-4-yl)-3H-imidazo[4,5-b]pyridin-3- | | |
| | yl)benzyl)piperidin-4-yl)amino)pyrimidine-2-carbonitrile | | |
| 360 | 2-((1-(4-(2-(2-aminopyridin-3-yl)-5-(1-methyl-2-oxo-1,2- | 610.1 | ++ |
| | dihydropyridin-4-yl)-3H-imidazo[4,5-b]pyridin-3- | | |
| | yl)benzyl)piperidin-4-yl)amino)pyrimidine-4-carbonitrile | | |
| 361 | 4-(7-(4-(2-(2-aminopyridin-3-yl)-5-phenyl-3H-imidazo[4,5- | 605.4 | ++++ |
| | b]pyridin-3-yl)benzyl)-2,7-diazaspiro[3.5]nonan-2- | | |
| | yl)pyrimidine-2-carbonitrile | | |
| 362 | 4-((7-(4-(2-(2-aminopyridin-3-yl)-5-phenyl-3H-imidazo[4,5- | 619.4 | ++++ |
| | b]pyridin-3-yl)benzyl)-7-azaspiro[3.5]nonan-2- | | |
| | yl)amino)pyrimidine-2-carbonitrile | | |
| 363 | 4-(8-(4-(2-(2-aminopyridin-3-yl)-5-phenyl-3H-imidazo[4,5- | 620.3 | +++ |
| | b]pyridin-3-yl)benzyl)-2,8-diazaspiro[4.5]decan-2-yl)-1,3,5- | | |
| | triazine-2-carbonitrile | | |
| 364 | 4-(9-(4-(2-(2-aminopyridin-3-yl)-5-phenyl-3H-imidazo[4,5- | 633.5 | +++ |
| | b]pyridin-3-yl)benzyl)-3,9-diazaspiro[5.5]undecan-3- | | |
| | yl)pyrimidine-2-carbonitrile | | |
| 365 | 4-(9-(4-(2-(2-aminopyridin-3-yl)-5-phenyl-3H-imidazo[4,5- | 634.3 | +++ |
| | b]pyridin-3-yl)benzyl)-3,9-diazaspiro[5.5]undecan-3-yl)-1,3,5- | | |
| | triazine-2-carbonitrile | | |
| 366 | 4-(6-(4-(2-(2-aminopyridin-3-yl)-5-phenyl-3H-imidazo[4,5- | 577.3 | ++++ |
| | b]pyridin-3-yl)benzyl)-2,6-diazaspiro[3.3]heptan-2- | | |
| | yl)pyrimidine-2-carbonitrile | | |
| 367 | 4-((3aS,6aS)-5-(4-(2-(2-aminopyridin-3-yl)-5-phenyl-3H- | 591.3 | ++++ |
| | imidazo[4,5-b]pyridin-3-yl)benzyl)hexahydropyrrolo[3,4- | | |
| | c]pyrrol-2(1H)-yl)pyrimidine-2-carbonitrile | | |
| 368 | 4-(4-(4-(2-(2-aminopyridin-3-yl)-5-(4-fluorophenyl)-3H- | 584.4 | ++++ |
| | imidazo[4,5-b]pyridin-3-yl)benzyl)piperazin-1-yl)-1,3,5- | | |
| | triazine-2-carbonitrile | | |
| 369 | 4-(4-(4-(2-(2-aminopyridin-3-yl)-5-(4-chlorophenyl)-3H- | 600.4 | +++ |
| | imidazo[4,5-b]pyridin-3-yl)benzyl)piperazin-1-yl)-1,3,5- | | |
| | triazine-2-carbonitrile | | |
| 370 | 4-((1-(4-(2-(2-aminopyridin-3-yl)-5-(4-fluorophenyl)-3H- | 597.4 | ++++ |
| | imidazo[4,5-b]pyridin-3-yl)benzyl)piperidin-4- | | |
| | yl)amino)pyrimidine-2-carbonitrile | | |
| 371 | 2-((1-(4-(2-(2-aminopyridin-3-yl)-5-(4-chlorophenyl)-3H- | 613.4 | ++ |
| | imidazo[4,5-b]pyridin-3-yl)benzyl)piperidin-4- | | |
| | yl)amino)pyrimidine-4-carbonitrile | | |
| 372 | 2-((1-(4-(2-(2-aminopyridin-3-yl)-5-(3-chlorophenyl)-3H- | 613.3 | +++ |
| | imidazo[4,5-b]pyridin-3-yl)benzyl)piperidin-4- | | |
| | yl)amino)pyrimidine-4-carbonitrile | | |
| 373 | 6-((1-(4-(2-(2-aminopyridin-3-yl)-5-(3-chlorophenyl)-3H- | 613.4 | +++ |
| | imidazo[4,5-b]pyridin-3-yl)benzyl)piperidin-4- | | |
| | yl)amino)pyrimidine-4-carbonitrile | | |
| 374 | 6-(4-(4-(2-(2-aminopyridin-3-yl)-5-(3-chlorophenyl)-3H- | 599.4 | +++ |
| | imidazo[4,5-b]pyridin-3-yl)benzyl)piperazin-1-yl)pyrimidine- | | |
| | 4-carbonitrile | | |
| 375 | 4-((1S,4S)-5-(4-(2-(2-aminopyridin-3-yl)-5-phenyl-3H- | 577.3 | +++ |
| | imidazo[4,5-b]pyridin-3-yl)benzyl)-2,5- | | |
| | diazabicyclo[2.2.1]heptan-2-yl)pyrimidine-2-carbonitrile | | |
| 376 | 6-((1S,4S)-5-(4-(2-(2-aminopyridin-3-yl)-5-phenyl-3H- | 577.3 | ++ |
| | imidazo[4,5-b]pyridin-3-yl)benzyl)-2,5- | | |
| | diazabicyclo[2.2.1]heptan-2-yl)pyrimidine-4-carbonitrile | | |
| 377 | 2-((18,4S)-5-(4-(2-(2-aminopyridin-3-yl)-5-phenyl-3H- | 577.3 | ++ |
| | imidazo[4,5-b]pyridin-3-yl)benzyl)-2,5- | | |
| | diazabicyclo[2.2.1]heptan-2-yl)pyrimidine-4-carbonitrile | | |
| 378 | 4-((2S,6R)-4-(4-(2-(2-aminopyridin-3-yl)-5-phenyl-3H- | 593.4 | ++ |
| | imidazo[4,5-b]pyridin-3-yl)benzyl)-2,6-dimethylpiperazin-1- | | |
| | yl)pyrimidine-2-carbonitrile | | |
| 379 | 2-((2S,6R)-4-(4-(2-(2-aminopyridin-3-yl)-5-phenyl-3H- | 593.2 | + |
| | imidazo[4,5-b]pyridin-3-yl)benzyl)-2,6-dimethylpiperazin-1- | | |
| | yl)pyrimidine-4-carbonitrile | | |
| 380 | 6-((2S,6R)-4-(4-(2-(2-aminopyridin-3-yl)-5-phenyl-3H- | 593.4 | + |
| | imidazo[4,5-b]pyridin-3-yl)benzyl)-2,6-dimethylpiperazin-1- | | |
| | yl)pyrimidine-4-carbonitrile | | |
| 381 | 4-((1-(4-(2-(2-aminopyridin-3-yl)-5-(2-oxo-1,2- | 596.3 | ++++ |
| | dihydropyridin-3-yl)-3H-imidazo[4,5-b]pyridin-3- | | |
| | yl)benzyl)piperidin-4-yl)amino)pyrimidine-2-carbonitrile | | |
| 382 | 4-((1-(4-(2-(2-aminopyridin-3-yl)-5-(4-chlorophenyl)-3H- | 614.4 | +++ |
| | imidazo[4,5-b]pyridin-3-yl)benzyl)piperidin-4-yl)amino)- | | |
| | 1,3,5-triazine-2-carbonitrile | | |
| 383 | 4-(4-(4-(2-(2-aminopyridin-3-yl)-5-(4-cyanopyridin-3-yl)-3H- | 591.4 | ++++ |
| | imidazo[4,5-b]pyridin-3-yl)benzyl)piperazin-1-yl)pyrimidine- | | |
| | 2-carbonitrile | | |
| 384 | 4-((1-(4-(2-(2-aminopyridin-3-yl)-5-(4-cyanopyridin-3-yl)-3H- | 605.4 | +++ |
| | imidazo[4,5-b]pyridin-3-yl)benzyl)piperidin-4- | | |
| | yl)amino)pyrimidine-2-carbonitrile | | |
| 385 | 4-((1-((4-(2-(2-aminopyridin-3-yl)-3H-imidazo[4,5-b]pyridin- | 505.2 | +++ |
| | 3-yl)phenyl)methyl-d2)piperidin-4-yl)amino)pyrimidine-2- | | |
| | carbonitrile | | |
| 386 | 4-(4-(4-(2-(2-aminopyridin-3-yl)-5-(4-methyl-3-oxo-1,4- | 615.3 | +++ |
| | diazepan-1-yl)-3H-imidazo[4,5-b]pyridin-3- | | |
| | yl)benzyl)piperazin-1-yl)pyrimidine-2-carbonitrile | | |
| 387 | 4-((1-(4-(2-(2-aminopyridin-3-yl)-5-(3-oxomorpholino)-3H- | 602.4 | ++ |
| | imidazo[4,5-b]pyridin-3-yl)benzyl)piperidin-4- | | |
| | yl)amino)pyrimidine-2-carbonitrile | | |
| 388 | 4-((1-(4-(2-(2-aminopyridin-3-yl)-5-cyclopentyl-3H- | 571.2 | ++++ |
| | imidazo[4,5-b]pyridin-3-yl)benzyl)piperidin-4- | | |
| | yl)amino)pyrimidine-2-carbonitrile | | |
| 389 | 4-((1-(4-(2-(2-aminopyridin-3-yl)-5-(dimethylamino)-3H- | 546.4 | ++++ |
| | imidazo[4,5-b]pyridin-3-yl)benzyl)piperidin-4- | | |
| | yl)amino)pyrimidine-2-carbonitrile | | |
| 390 | 4-((1-(4-(2-(2-aminopyridin-3-yl)-3H-imidazo[4,5-b]pyridin-3- | 517.2 | ++++ |
| | yl)benzyl)piperidin-4-yl)(methyl)amino)pyrimidine-2- | | |
| | carbonitrile | | |
| 391 | 4-((1-(4-(2-(2-aminopyridin-3-yl)-5-phenyl-3H-imidazo[4,5- | 580.2 | ++++ |
| | b]pyridin-3-yl)benzyl)piperidin-4-yl)amino)-1,3,5-triazine-2- | | |
| | carbonitrile | | |
| 392 | 4-((1-(4-(2-(2-aminopyridin-3-yl)-3H-imidazo[4,5-b]pyridin-3- | 517.2 | ++ |
| | yl)benzyl)-4-methylpiperidin-4-yl)amino)pyrimidine-2- | | |
| | carbonitrile | | |
| 393 | 4-((1-(4-(2-(2-aminopyridin-3-yl)-5-(1-methyl-6-oxo-1,6- | 610.5 | + |
| | dihydropyridin-2-yl)-3H-imidazo[4,5-b]pyridin-3- | | |
| | yl)benzyl)piperidin-4-yl)amino)pyrimidine-2-carbonitrile | | |
| 394 | 4-(4-(4-(2-(2-aminopyridin-3-yl)-5-(1-methyl-6-oxo-1,6- | 596.1 | + |
| | dibydropyridin-2-yl)-3H-imidazo[4,5-b]pyridin-3- | | |
| | yl)benzyl)piperazin-1-yl)pyrimidine-2-carbonitrile | | |
| 395 | 4-((1-(4-(2-(2-aminopyridin-3-yl)-5-(3-methoxyphenyl)-3H- | 609.5 | ++++ |
| | imidazo[4,5-b]pyridin-3-yl)benzyl)piperidin-4- | | |
| | yl)amino)pyrimidine-2-carbonitrile | | |
| 396 | 4-((1-(4-(2-(2-aminopyridin-3-yl)-5-(tert-butyl)-3H- | 559.2 | +++ |
| | imidazo[4,5-b]pyridin-3-yl)benzyl)piperidin-4- | | |
| | yl)amino)pyrimidine-2-carbonitrile | | |
| 397 | 4-((1-(4-(2-(2-aminopyridin-3-yl)-5-ethyl-3H-imidazo[4,5- | 531.2 | ++++ |
| | b]pyridin-3-yl)benzyl)piperidin-4-yl)amino)pyrimidine-2- | | |
| | carbonitrile | | |
| 398 | 2-(2-aminopyridin-3-yl)-3-(4-(4-(2-cyanopyrimidin-4- | 560.2 | ++++ |
| | yl)amino)piperidin-1-yl)methyl)phenyl)-N-methyl-3H- | | |
| | imidazo[4,5-b]pyridine-5-carboxamide | | |
| 399 | 4-(4-(4-(2-(2-aminopyridin-3-yl)-5-(3-cyanopyridin-4-yl)-3H- | 591.3 | +++ |
| | imidazo[4,5-b]pyridin-3-yl)benzyl)piperazin-1-yl)pyrimidine- | | |
| | 2-carbonitrile | | |
| 400 | 4-((1-(4-(2-(2-aminopyridin-3-yl)-5-(3-cyanopyridin-4-yl)-3H- | 605.4 | ++++ |
| | imidazo[4,5-b]pyridin-3-yl)benzyl)piperidin-4- | | |
| | yl)amino)pyrimidine-2-carbonitrile | | |
| 401 | 4-((1-(4-(2-(2-aminopyridin-3-yl)-5-fluoro-3H-imidazo[4,5- | 521.4 | +++ |
| | b]pyridin-3-yl)benzyl)piperidin-4-yl)amino)pyrimidine-2- | | |
| | carbonitrile | | |
| 402 | 4-((1-(4-(2-(2-aminopyridin-3-yl)-6-fluoro-3H-imidazo[4,5- | 521.1 | ++++ |
| | b]pyridin-3-yl)benzyl)piperidin-4-yl)amino)pyrimidine-2- | | |
| | carbonitrile | | |
| 403 | 4-((1-(4-(2-(2-aminopyridin-3-yl)-5-(2-oxooxazolidin-3-yl)- | 588.1 | ++++ |
| | 3H-imidazo[4,5-b]pyridin-3-yl)benzyl)piperidin-4- | | |
| | yl)amino)pyrimidine-2-carbonitrile | | |
| 404 | 4-((1-(4-(2-(2-aminopyridin-3-yl)-5-(6-fluoropyridin-3-yl)-3H- | 598.4 | ++++ |
| | imidazo[4,5-b]pyridin-3-yl)benzyl)piperidin-4- | | |
| | yl)amino)pyrimidine-2-carbonitrile | | |
| 405 | 4-((1-(4-(2-(2-aminopyridin-3-yl)-5-chloro-3H-imidazo[4,5- | 537.2 | ++++ |
| | bjpyridin-3-yl)benzyl)piperidin-4-yl)amino)pyrimidine-2- | | |
| | carbonitrile | | |
| 406 | 2-(2-aminopyridin-3-yl)-3-(4-((4-((2-cyanopyrimidin-4- | 528.3 | ++++ |
| | yl)amino)piperidin-1-yl)methyl)phenyl)-3H-imidazo[4,5- | | |
| | b]pyridine-5-carbonitrile | | |
| 407 | 4-(4-(4-(2-(2-aminopyridin-3-yl)-5-(3-cyanopyridin-2-yl)-3H- | 591.1 | ++++ |
| | imidazo[4,5-b]pyridin-3-yl)benzyl)piperazin-1-yl)pyrimidine- | | |
| | 2-carbonitrile | | |
| 408 | 4-((1-(4-(2-(2-aminopyridin-3-yl)-5-(3-cyanopyridin-2-yl)-3H- | 605.4 | ++++ |
| | imidazo[4,5-b]pyridin-3-yl)benzyl)piperidin-4- | | |
| | yl)amino)pyrimidine-2-carbonitrile | | |
| 409 | 4-((1-(4-(2-(2-aminopyridin-3-yl)-5-(2-cyanopyridin-3-yl)-3H- | 605.4 | ++++ |
| | imidazo[4,5-b]pyridin-3-yl)benzyl)piperidin-4- | | |
| | yl)amino)pyrimidine-2-carbonitrile | | |
| 410 | 4-((1-(4-(2-(2-aminopyridin-3-yl)-5-(trifluoromethyl)-3H- | 571.3 | ++++ |
| | imidazo[4,5-b]pyridin-3-yl)benzyl)piperidin-4- | | |
| | yl)amino)pyrimidine-2-carbonitrile | | |
| 411 | 2-(2-aminopyridin-3-yl)-3-(4-((4-((2-cyanopyrimidin-4- | 528.4 | +++ |
| | yl)amino)piperidin-1-yl)methyl)phenyl)-3H-imidazo[4,5- | | |
| | b]pyridine-6-carbonitrile | | |
| 412 | 4-((1-(4-(2-(2-aminopyridin-3-yl)-5,6-dimethyl-3H- | 531.4 | ++++ |
| | imidazo[4,5-b]pyridin-3-yl)benzyl)piperidin-4- | | |
| | yl)amino)pyrimidine-2-carbonitrile | | |
| 413 | 4-((1-(4-(2-(2-aminopyridin-3-yl)-1H-imidazo[4,5-b]pyrazin- | 504.1 | +++ |
| | 1-yl)benzyl)piperidin-4-yl)amino)pyrimidine-2-carbonitrile | | |
| 414 | methyl 2-(2-aminopyridin-3-yl)-3-(4-((4-((2-cyanopyrimidin- | 561.3 | ++++ |
| | 4-yl)amino)piperidin-1-yl)methyl)phenyl)-3H-imidazo[4,5- | | |
| | b]pyridine-5-carboxylate | | |
| 415 | 2-(2-aminopyridin-3-yl)-3-(4-((4-(2-cyanopyrimidin-4- | 547.4 | + |
| | yl)amino)piperidin-1-yl)methyl)phenyl)-3H-imidazo[4,5- | | |
| | b]pyridine-5-carboxylic acid | | |
| 416 | 4-((1-(4-(2-(2-aminopyridin-3-yl)-5-(fluoromethoxy)-3H- | 551.1 | ++++ |
| | imidazo[4,5-b]pyridin-3-yl)benzyl)piperidin-4- | | |
| | yl)amino)pyrimidine-2-carbonitrile | | |
| 417 | 4-((1-(4-(2-(2-aminopyridin-3-yl)-5-(tetrahydrofuran-3-yl)-3H- | 573.4 | ++++ |
| | imidazo[4,5-b]pyridin-3-yl)benzyl)piperidin-4- | | |
| | yl)amino)pyrimidine-2-carbonitrile | | |
| 418 | 4-((1-(4-(2-(2-aminopyridin-3-yl)-5-(5-cyanopyridin-2-yl)-3H- | 605.0 | +++ |
| | imidazo[4,5-b]pyridin-3-yl)benzyl)piperidin-4- | | |
| | yl)amino)pyrimidine-2-carbonitrile | | |
| 419 | 4-((1-(4-(2-(2-aminopyridin-3-yl)-5-(6-cyanopyridin-3-yl)-3H- | 605.1 | ++++ |
| | imidazo[4,5-b]pyridin-3-yl)benzyl)piperidin-4- | | |
| | yl)amino)pyrimidine-2-carbonitrile | | |
| 420 | 4-((1-(4-(2-(2-aminopyridin-3-yl)-5-(oxetan-3-yl)-3H- | 559.1 | ++++ |
| | imidazo[4,5-b]pyridin-3-yl)benzyl)piperidin-4- | | |
| | yl)amino)pyrimidine-2-carbonitrile | | |
| 421 | 4-((1-(4-(2-(2-aminopyridin-3-yl)-5-(4-methoxyphenyl)-3H- | 609.2 | ++++ |
| | imidazo[4,5-b]pyridin-3-yl)benzyl)piperidin-4- | | |
| | yl)amino)pyrimidine-2-carbonitrile | | |
| 422 | 4-((1-(4-(2-(2-aminopyridin-3-yl)-5-(2-methoxyphenyl)-3H- | 609.1 | ++++ |
| | imidazo[4,5-b]pyridin-3-yl)benzyl)piperidin-4- | | |
| | yl)amino)pyrimidine-2-carbonitrile | | |
| 423 | 4-((1-(4-(2-(2-aminopyridin-3-yl)-5-(trifluoromethoxy)-3H- | 587.1 | ++ |
| | imidazo[4,5-b]pyridin-3-yl)benzyl)piperidin-4- | | |
| | yl)amino)pyrimidine-2-carbonitrile | | |
| 424 | 4-(4-(4-(2-(2-aminopyridin-3-yl)-5-(2-cyanopyridin-3-yl)-3H- | 591.4 | +++ |
| | imidazo[4,5-b]pyridin-3-yl)benzyl)piperazin-1-yl)pyrimidine- | | |
| | 2-carbonitrile | | |
| 425 | 4-((1-(4-(2-(2-aminopyridin-3-yl)-5-isopropoxy-3H- | 561.1 | ++++ |
| | imidazo[4,5-b]pyridin-3-yl)benzyl)piperidin-4- | | |
| | yl)amino)pyrimidine-2-carbonitrile | | |
| 426 | 4-((4-(4-(2-(2-aminopyridin-3-yl)-5-phenyl-3H-imidazo[4,5- | 580.4 | ++++ |
| | b]pyridin-3-yl)benzyl)piperazin-1-yl)amino)pyrimidine-2- | | |
| | carbonitrile | | |
| 427 | 4-((1-(4-(2-(2-aminopyridin-3-yl)-5-(methoxy-d3)-3H- | 536.1 | ++++ |
| | imidazo[4,5-b]pyridin-3-yl)benzyl)piperidin-4- | | |
| | yl)amino)pyrimidine-2-carbonitrile | | |
| 428 | 4-((1-(4-(2-(2-aminopyridin-3-yl)-5-(6-cyanopyridin-2-yl)-3H- | 605.1 | +++ |
| | imidazo[4,5-b]pyridin-3-yl)benzyl)piperidin-4- | | |
| | yl)amino)pyrimidine-2-carbonitrile | | |
| 429 | 4-((1-(4-(2-(2-aminopyridin-3-yl)-5-(2-cyanopyridin-4-yl)-3H- | 605.2 | ++++ |
| | imidazo[4,5-b]pyridin-3-yl)benzyl)piperidin-4- | | |
| | yl)amino)pyrimidine-2-carbonitrile | | |
| 430 | 4-((1-(4-(2-(2-aminopyridin-3-yl)-5-(5-cyanopyridin-3-yl)-3H- | 605.3 | ++++ |
| | imidazo[4,5-b]pyridin-3-yl)benzyl)piperidin-4- | | |
| | yl)amino)pyrimidine-2-carbonitrile | | |
| 431 | 4-((1-(4-(2-(2-aminopyridin-3-yl)-5-(pyridin-3-yl)-3H- | 580.3 | ++++ |
| | imidazo[4,5-b]pyridin-3-yl)benzyl)piperidin-4- | | |
| | yl)amino)pyrimidine-2-carbonitrile | | |
| 432 | 4-(4-(4-(2-(2-aminopyridin-3-yl)-5-(pyridin-3-yl)-3H- | 566.3 | ++++ |
| | imidazo[4,5-b]pyridin-3-yl)benzyl)piperazin-1-yl)pyrimidine- | | |
| | 2-carbonitrile | | |
| 433 | 4-((1-(4-(2-(2-aminopyridin-3-yl)-5-(pyridin-4-yl)-3H- | 580.4 | ++++ |
| | imidazo[4,5-b]pyridin-3-yl)benzyl)piperidin-4- | | |
| | yl)amino)pyrimidine-2-carbonitrile | | |
| 434 | 4-((7-(4-(2-(2-aminopyridin-3-yl)-5-morpholino-3H- | 629.3 | +++ |
| | imidazo[4,5-b]pyridin-3-yl)benzyl)-7-azaspiro[3.5]nonan-2- | | |
| | yl)amino)-1,3,5-triazine-2-carbonitrile | | |
| 435 | 4-((1-(4-(2-(2-aminopyridin-3-yl)-5-(methylthio)-3H- | 571.0 | ++++ |
| | imidazo[4,5-b]pyridin-3-yl)benzyl)piperidin-4- | [M + Na]⁺. | |
| | yl)amino)pyrimidine-2-carbonitrile | | |
| 436 | 4-((1-(4-(2-(2-aminopyridin-3-yl)-5-(methylsulfonyl)-3H- | 581.3 | ++ |
| | imidazo[4,5-b]pyridin-3-yl)benzyl)piperidin-4- | | |
| | yl)amino)pyrimidine-2-carbonitrile | | |
| 437 | 4-((1-(4-(2-(2-aminopyridin-3-yl)-5-(4-methoxypyridin-2-yl)- | 610.2 | ++++ |
| | 3H-imidazo[4,5-b]pyridin-3-yl)benzyl)piperidin-4- | | |
| | yl)amino)pyrimidine-2-carbonitrile | | |
| 438 | 4-((1-(4-(2-(2-aminopyridin-3-yl)-5-(1-methyl-1H-1,2,3- | 584.2 | ++++ |
| | triazol-4-yl)-3H-imidazo[4,5-b]pyridin-3-yl)benzyl)piperidin- | | |
| | 4-yl)amino)pyrimidine-2-carbonitrile | | |
| 439 | 4-((1-(4-(2-(2-aminopyridin-3-yl)-5-(4-fluoro-3- | 627.3 | ++++ |
| | methoxyphenyl)-3H-imidazo[4,5-b]pyridin-3- | | |
| | yl)benzyl)piperidin-4-yl)amino)pyrimidine-2-carbonitrile | | |
| 440 | N-(3-(2-(2-aminopyridin-3-yl)-3-(4-((4-((2-cyanopyrimidin-4- | 636.2 | ++++ |
| | yl)amino)piperidin-1-yl)methyl)phenyl)-3H-imidazo[4,5- | | |
| | b]pyridin-5-yl)phenyl)acetamide | | |
| 441 | 4-((1-(4-(2-(2-aminopyridin-3-yl)-5-(4-fluorophenyl)-3H- | 614.7 | ++++ |
| | imidazo[4,5-b]pyridin-3-yl)benzyl)piperidin-4-yl)(methyl- | | |
| | d3)amino)pyrimidine-2-carbonitrile | | |
| 442 | 4-((1-((4-(2-(2-aminopyridin-3-yl)-5-(4-fluorophenyl)-3H- | 616.5 | ++++ |
| | imidazo(4,5-b]pyridin-3-yl)phenyl)methyl-d2)piperidin-4- | | |
| | yl)(methyl-d3)amino)pyrimidine-2-carbonitrile | | |
| 443 | 4-((1-(4-(2-(2-aminopyridin-3-yl)-5-(4-fluorophenyl)-3H- | 569.3 | ++++ |
| | imidazo[4,5-b]pyridin-3-yl)benzyl)azetidin-3- | | |
| | yl)amino)pyrimidine-2-carbonitrile | | |
| 444 | 4-((7-(4-(2-(2-aminopyridin-3-yl)-5-(4-fluorophenyl)-3H- | 637.4 | +++ |
| | imidazo[4,5-b]pyridin-3-yl)benzyl)-7-azaspiro[3.5]nonan-2- | | |
| | yl)amino)pyrimidine-2-carbonitrile | | |
| 445 | 4-((2-(4-(2-(2-aminopyridin-3-yl)-5-(4-fluorophenyl)-3H- | 609.3 | ++++ |
| | imidazo[4,5-b]pyridin-3-yl)benzyl)-2-azaspiro[3.3]heptan-6- | | |
| | yl)amino)pyrimidine-2-carbonitrile | | |
| 446 | 4-((2-(4-(2-(2-aminopyridin-3-yl)-5-(4-fluorophenyl)-3H- | 637.4 | +++ |
| | imidazo[4,5-b]pyridin-3-yl)benzyl)-2-azaspiro[3.5]nonan-7- | | |
| | yl)amino)pyrimidine-2-carbonitrile | | |
| 447 | 4-(6-(4-(2-(2-aminopyridin-3-yl)-5-(4-fluorophenyl)-3H- | 595.3 | ++++ |
| | imidazo[4,5-b]pyridin-3-yl)benzyl)-2,6-diazaspiro[3.3]heptan- | | |
| | 2-yl)pyrimidine-2-carbonitrile | | |
| 448 | 4-(8-(4-(2-(2-aminopyridin-3-yl)-5-(4-fluorophenyl)-3H- | 609.2 | +++ |
| | imidazo[4,5-b]pyridin-3-yl)benzyl)-3,8- | | |
| | diazabicyclo[3.2.1]octan-3-yl)pyrimidine-2-carbonitrile | | |
| 449 | 4-((3aR,6aS)-5-(4-(2-(2-aminopyridin-3-yl)-5-(4- | 609.3 | +++ |
| | fluorophenyl)-3H-imidazo[4,5-b]pyridin-3- | | |
| | yl)benzyl)hexahydropyrrolo[3,4-c]pyrrol-2(1H)-yl)pyrimidine- | | |
| | 2-carbonitrile | | |
| 450 | 4-(3aS,6aS)-5-(4-(2-(2-aminopyridin-3-yl)-5-(4- | 609.4 | +++ |
| | fluorophenyl)-3H-imidazo[4,5-b]pyridin-3- | | |
| | yl)benzyl)hexahydropyrrolo[3,4-c]pyrrol-2(1H)-yl)pyrimidine- | | |
| | 2-carbonitrile | | |
| 451 | 4-((1-(4-(2-(2-aminopyridin-3-yl)-5-(4-fluorophenyl)-3H- | 598.7 | ++++ |
| | imidazo[4,5-b]pyridin-3-yl)benzyl)piperidin-4-yl-4- | | |
| | d)amino)pyrimidine-2-carbonitrile | | |
| 452 | 4-((1-(4-(2-(2-aminopyridin-3-yl)-5,6-dimethyl-3H- | 532.7 | ++++ |
| | imidazo[4,5-b]pyridin-3-yl)benzyl)piperidin-4-yl-4- | | |
| | d)amino)pyrimidine-2-carbonitrile | | |
| 453 | 4-((1-(4-(2-(2-aminopyridin-3-yl)-5-(cyclopent-1-en-1-yl)-3H- | 569.3 | ++++ |
| | imidazo[4,5-b]pyridin-3-yl)benzyl)piperidin-4- | | |
| | yl)amino)pyrimidine-2-carbonitrile | | |
| 454 | 4-((1-(4-(2-(2-aminopyridin-3-yl)-5-vinyl-3H-imidazo[4,5- | 529.2 | ++++ |
| | b]pyridin-3-yl)benzyl)piperidin-4-yl)amino)pyrimidine-2- | | |
| | carbonitrile | | |
| 455 | 4-((1-(4-(2-(2-aminopyridin-3-yl)-5,6-dimethyl-3H- | 548.3 | ++++ |
| | imidazo[4,5-b]pyridin-3-yl)benzyl)piperidin-4-yl)(methyl- | | |
| | d3)amino)pyrimidine-2-carbonitrile | | |
| 456 | 4-((1-(4-(2-(2-aminopyridin-3-yl)-5-(5-fluoropyridin-2-yl)-3H- | 598.2 | ++++ |
| | imidazo[4,5-b]pyridin-3-yl)benzyl)piperidin-4- | | |
| | yl)amino)pyrimidine-2-carbonitrile | | |
| 457 | 4-((1-(4-(2-(2-aminopyridin-3-yl)-5-(methylamino)-3H- | 532.2 | +++ |
| | imidazo[4,5-b]pyridin-3-yl)benzyl)piperidin-4- | | |
| | yl)amino)pyrimidine-2-carbonitrile | | |
| 458 | 4-((1-(4-(2-(2-aminopyridin-3-yl)-5-(bis(methyl-d3)amino)- | 552.0 | ++++ |
| | 3H-imidazo[4,5-b]pyridin-3-yl)benzyl)piperidin-4- | | |
| | yl)amino)pyrimidine-2-carbonitrile | | |
| 459 | 4-((1-(4-(2-(2-aminopyridin-3-yl)-5-((methyl-d3)amino)-3H- | 535.1 | ++++ |
| | imidazo[4,5-b]pyridin-3-yl)benzyl)piperidin-4- | | |
| | yl)amino)pyrimidine-2-carbonitrile | | |
| 460 | 4-((1-(4-(2-(2-aminopyridin-3-yl)-5-(azetidin-1-yl)-3H- | 558.5 | ++++ |
| | imidazo[4,5-b]pyridin-3-yl)benzyl)piperidin-4- | | |
| | yl)amino)pyrimidine-2-carbonitrile | | |
| 461 | 4-((1-(4-(2-(2-aminopyridin-3-yl)-5-(isoxazol-4-yl)-3H- | 570.3 | ++++ |
| | imidazo[4,5-b]pyridin-3-yl)benzyl)piperidin-4- | | |
| | yl)amino)pyrimidine-2-carbonitrile | | |
| 462 | 4-((1-(4-(2-(2-aminopyridin-3-yl)-5-(oxazol-5-yl)-3H- | 570.2 | ++++ |
| | imidazo[4,5-b]pyridin-3-yl)benzyl)piperidin-4- | | |
| | yl)amino)pyrimidine-2-carbonitrile | | |
| 463 | 4-((1-(4-(2-(2-aminopyridin-3-yl)-5-(5-methylisoxazol-4-yl)- | 584.3 | ++++ |
| | 3H-imidazo[4,5-b]pyridin-3-yl)benzyl)piperidin-4- | | |
| | yl)amino)pyrimidine-2-carbonitrile | | |
| 464 | 4-((1-(4-(2-(2-aminopyridin-3-yl)-5-(3-methylisoxazol-4-yl)- | 584.4 | ++++ |
| | 3H-imidazo[4,5-b]pyridin-3-yl)benzyl)piperidin-4- | | |
| | yl)amino)pyrimidine-2-carbonitrile | | |
| 465 | 4-((1-(4-(2-(2-aminopyridin-3-yl)-5-(3-methyl-1H-pyrazol-5- | 583.3 | ++++ |
| | yl)-3H-imidazo[4,5-b]pyridin-3-yl)benzyl)piperidin-4- | | |
| | yl)amino)pyrimidine-2-carbonitrile | | |
| 466 | 4-((1-(4-(2-(2-aminopyridin-3-yl)-5-(1-ethyl-1H-pyrazol-4-yl)- | 597.4 | ++++ |
| | 3H-imidazo[4,5-b]pyridin-3-yl)benzyl)piperidin-4- | | |
| | yl)amino)pyrimidine-2-carbonitrile | | |
| 467 | 4-((1-(4-(2-(2-aminopyridin-3-yl)-5-(1-methyl-1H-pyrazol-5- | 583.4 | ++++ |
| | yl)-3H-imidazo[4,5-b]pyridin-3-yl)benzyl)piperidin-4- | | |
| | yl)amino)pyrimidine-2-carbonitrile | | |
| 468 | 4-((1-(4-(2-(2-aminopyridin-3-yl)-5-(3-(2-oxopyrrolidin-1- | 662.4 | ++++ |
| | yl)phenyl)-3H-imidazo[4,5-b]pyridin-3-yl)benzyl)piperidin-4- | | |
| | yl)amino)pyrimidine-2-carbonitrile | | |
| 469 | 4-((1-(4-(2-(2-aminopyridin-3-yl)-5-(4-fluoro-2- | 627.5 | ++++ |
| | methoxyphenyl)-3H-imidazo[4,5-b]pyridin-3- | | |
| | yl)benzyl)piperidin-4-yl)amino)pyrimidine-2-carbonitrile | | |
| 470 | 4-((1-(4-(2-(2-aminopyridin-3-yl)-5-(2- | 647.3 | ++ |
| | (trifluoromethyl)phenyl)-3H-imidazo[4,5-b]pyridin-3- | | |
| | yl)benzyl)piperidin-4-yl)amino)pyrimidine-2-carbonitrile | | |
| 471 | 4-((1-(4-(2-(2-aminopyridin-3-yl)-5-(4- | 647.3 | +++ |
| | (trifluoromethyl)phenyl)-3H-imidazo[4,5-b]pyridin-3- | | |
| | yl)benzyl)piperidin-4-yl)amino)pyrimidine-2-carbonitrile | | |
| 472 | 4-((1-(4-(2-(2-aminopyridin-3-yl)-5-(2-methyl-2H-1,2,3- | 584.3 | ++++ |
| | triazol-4-yl)-3H-imidazo[4,5-b]pyridin-3-yl)benzyl)piperidin- | | |
| | 4-yl)amino)pyrimidine-2-carbonitrile | | |
| 473 | 4-((1-(4-(2-(2-aminopyridin-3-yl)-5-(3,5-dimethylisoxazol-4- | 598.4 | ++++ |
| | yl)-3H-imidazo[4,5-b]pyridin-3-yl)benzyl)piperidin-4- | | |
| | yl)amino)pyrimidine-2-carbonitrile | | |
| 474 | 4-((1-(4-(2-(2-aminopyridin-3-yl)-5-(1-(difluoromethyl)-1H- | 619.3 | ++++ |
| | pyrazol-4-yl)-3H-imidazo[4,5-b]pyridin-3-yl)benzyl)piperidin- | | |
| | 4-yl)amino)pyrimidine-2-carbonitrile | | |
| 475 | 4-((1-(4-(2-(2-aminopyridin-3-yl)-5-(2,4-difluorophenyl)-3H- | 615.3 | ++++ |
| | imidazo[4,5-b]pyridin-3-yl)benzyl)piperidin-4- | | |
| | yl)amino)pyrimidine-2-carbonitrile | | |
| 476 | 4-((1-(4-(2-(2-aminopyridin-3-yl)-5-(6-methoxypyridin-2-yl)- | 610.5 | ++++ |
| | 3H-imidazo[4,5-b]pyridin-3-yl)benzyl)piperidin-4- | | |
| | yl)amino)pyrimidine-2-carbonitrile | | |
| 477 | 4-((1-(4-(2-(2-aminopyridin-3-yl)-5-(2-methylpyridin-4-yl)- | 594.3 | ++++ |
| | 3H-imidazo[4,5-b]pyridin-3-yl)benzyl)piperidin-4- | | |
| | yl)amino)pyrimidine-2-carbonitrile | | |
| 478 | 4-((1-(4-(2-(2-aminopyridin-3-yl)-5-(5-methylpyridin-3-y])- | 594.4 | ++++ |
| | 3H-imidazo[4,5-b]pyridin-3-yl)benzyl)piperidin-4- | | |
| | yl)amino)pyrimidine-2-carbonitrile | | |
| 479 | 4-((1-(4-(2-(2-aminopyridin-3-yl)-5-(1-isopropyl-1H-pyrazol- | 611.3 | ++++ |
| | 4-yl)-3H-imidazo[4,5-b]pyridin-3-yl)benzyl)piperidin-4- | | |
| | yl)amino)pyrimidine-2-carbonitrile | | |
| 480 | 4-((1-(4-(2-(2-aminopyridin-3-yl)-5-(isothiazol-4-yl)-3H- | 586.3 | ++++ |
| | imidazo[4,5-b]pyridin-3-yl)benzyl)piperidin-4- | | |
| | yl)amino)pyrimidine-2-carbonitrile | | |
| 481 | 4-((1-(4-(2-(2-aminopyridin-3-yl)-5-(5-cyano-1-methyl-1H- | 607.3 | ++++ |
| | pyrrol-3-yl)-3H-imidazo[4,5-b]pyridin-3-yl)benzyl)piperidin- | | |
| | 4-yl)amino)pyrimidine-2-carbonitrile | | |
| 482 | 4-((1-(4-(2-(2-aminopyridin-3-yl)-5-(thiazol-4-yl)-3H- | 608.2 | ++++ |
| | imidazo[4,5-b]pyridin-3-yl)benzyl)piperidin-4- | [M + Na]⁺ | |
| | yl)amino)pyrimidine-2-carbonitrile | | |
| 483 | 4-((1-(4-(2-(2-aminopyridin-3-yl)-5-(isothiazol-5-yl)-3H- | 586.3 | ++++ |
| | imidazo[4,5-b]pyridin-3-yl)benzyl)piperidin-4- | | |
| | yl)amino)pyrimidine-2-carbonitrile | | |
| 484 | 4-((1-(4-(2-(2-aminopyridin-3-yl)-5-(1,5-dimethyl-1H-pyrazol- | 597.2 | ++++ |
| | 4-yl)-3H-imidazo[4,5-b]pyridin-3-yl)benzyl)piperidin-4- | | |
| | yl)amino)pyrimidine-2-carbonitrile | | |
| 485 | 4-((1-(4-(2-(2-aminopyridin-3-yl)-5-bromo-3H-imidazo[4,5- | 581.2, | ++++ |
| | b]pyridin-3-yl)benzyl)piperidin-4-yl)amino)pyrimidine-2- | 583.1 | |
| | carbonitrile | | |
| 486 | 4-((1-((4-(2-(2-aminopyridin-3-yl)-5-bromo-3H-imidazo[4,5- | 583.2, | ++++ |
| | b]pyridin-3-yl)phenyl)methyl-d2)piperidin-4- | 585.2 | |
| | yl)amino)pyrimidine-2-carbonitrile | | |
| 487 | 4-((1-(4-(2-(2-aminopyridin-3-yl)-5-(4-fluorophenyl)-3H- | 598.3 | ++++ |
| | imidazo[4,5-b]pyridin-3-yl)benzyl)piperidin-4-yl)amino)- | | |
| | 1,3,5-triazine-2-carbonitrile | | |
| 488 | 4-((1-((4-(2-(2-aminopyridin-3-yl)-5-(4-fluorophenyl)-3H- | 613.4 | ++++ |
| | imidazo[4,5-b]pyridin-3-yl)phenyl)methyl-d2)piperidin-4- | | |
| | yl)(methyl)amino)pyrimidine-2-carbonitrile | | |
| 489 | 4-((1-((4-(2-(2-aminopyridin-3-yl)-5-(4-fluorophenyl)-3H- | 599.3 | ++++ |
| | imidazo[4,5-b]pyridin-3-yl)phenyl)methyl-d2)piperidin-4- | | |
| | yl)amino)pyrimidine-2-carbonitrile | | |
| 490 | 4-(7-(4-(2-(2-aminopyridin-3-yl)-5-(4-fluorophenyl)-3H- | 623.3 | ++++ |
| | imidazo[4,5-b]pyridin-3-yl)benzyl)-2,7-diazaspiro[3.5]nonan- | | |
| | 2-yl)pyrimidine-2-carbonitrile | | |
| 491 | 4-(2-(4-(2-(2-aminopyridin-3-yl)-5-(4-fluorophenyl)-3H- | 623.3 | ++++ |
| | imidazo[4,5-b]pyridin-3-yl)benzyl)-2,7-diazaspiro[3.5]nonan- | | |
| | 7-yl)pyrimidine-2-carbonitrile | | |
| 492 | 4-((1-(4-(2-(2-aminopyridin-3-yl)-5-(methylsulfinyl)-3H- | 565.2 | ++ |
| | imidazo[4,5-b]pyridin-3-yl)benzyl)piperidin-4- | | |
| | yl)amino)pyrimidine-2-carbonitrile | | |
| 493 | 4-((1-(4-(2-(2-aminopyridin-3-yl)-5,7-dimethyl-3H- | 531.7 | ++++ |
| | imidazo[4,5-b]pyridin-3-yl)benzyl)piperidin-4- | | |
| | yl)amino)pyrimidine-2-carbonitrile | | |
| 494 | 4-((1-(4-(8-(2-aminopyridin-3-yl)-6-hydroxy-9H-purin-9- | 520.1 | + |
| | yl)benzyl)piperidin-4-yl)amino)pyrimidine-2-carbonitrile | | |
| 495 | 4-((1-(4-(2-(2-aminopyridin-3-yl)-5-(pyridin-2-yl)-3H- | 580.3 | ++++ |
| | imidazo[4,5-b]pyridin-3-yl)benzyl)piperidin-4- | | |
| | yl)amino)pyrimidine-2-carbonitrile | | |
| 496 | 4-((1-(4-(2-(2-aminopyridin-3-yl)-5-hydroxy-3H-imidazo[4,5- | 519.2 | +++ |
| | b]pyridin-3-yl)benzyl)piperidin-4-yl)amino)pyrimidine-2- | | |
| | carbonitrile | | |
| 497 | 4-(1-(4-(5-amino-2-(2-aminopyridin-3-yl)-3H-imidazo[4,5- | 518.2 | +++ |
| | b]pyridin-3-yl)benzyl)piperidin-4-yl)amino)pyrimidine-2- | | |
| | carbonitrile | | |
| 498 | 4-((1-(4-(2-(2-aminopyridin-3-yl)-5-(2-chloro-4-fluorophenyl)- | 631.3 | +++ |
| | 3H-imidazo[4,5-b]pyridin-3-yl)benzyl)piperidin-4- | | |
| | yl)amino)pyrimidine-2-carbonitrile | | |
| 499 | 4-((1-(4-(2-(2-aminopyridin-3-yl)-5-(4-fluorophenyl)-6- | 611.4 | ++++ |
| | methyl-3H-imidazo[4,5-b]pyridin-3-yl)benzyl)piperidin-4- | | |
| | yl)amino)pyrimidine-2-carbonitrile | | |
| 500 | 2-(2-aminopyridin-3-yl)-1-(4-((4-((2-cyanopyrimidin-4- | 527.4 | ++++ |
| | yl)amino)piperidin-1-yl)methyl)phenyl)-1H- | | |
| | benzo[d]imidazole-6-carbonitrile | | |
| 501 | 2-(2-aminopyridin-3-yl)-1-(4-((4-((2-cyanopyrimidin-4- | 527.1 | +++ |
| | yl)amino)piperidin-1-yl)methyl)phenyl)-1H- | | |
| | benzo[d]imidazole-5-carbonitrile | | |
| 502 | 4-((1-((4-(2-(2-aminopyridin-3-yl)-3H-imidazo[4,5-b]pyridin- | 522.3 | +++ |
| | 3-yl)phenyl)methyl-d2)piperidin-4-yl)(methyl- | | |
| | d3)amino)pyrimidine-2-carbonitrile | | |
| 503 | 4-((1-(4-(2-(2-aminopyridin-3-yl)-3H-imidazo[4,5-b]pyridin-3- | 504.2 | +++ |
| | yl)benzyl)piperidin-4-yl-4-d)amino)pyrimidine-2-carbonitrile | | |
| 504 | 4-((1-((4-(2-(2-aminopyridin-3-yl)-5,6-dimethyl-3H- | 533.2 | ++++ |
| | imidazo[4,5-b]pyridin-3-yl)phenyl)methyl-d2)piperidin-4- | | |
| | yl)amino)pyrimidine-2-carbonitrile | | |
| 505 | 4-((1-(4-(8-(2-aminopyridin-3-yl)-9H-purin-9- | 504.1 | ++ |
| | yl)benzyl)piperidin-4-yl)amino)pyrimidine-2-carbonitrile | | |
| 506 | (S)-4-((1-(4-(2-(2-aminopyridin-3-yl)-5-(tetrahydrofuran-3-yl)- | 573.3 | ++++ |
| | 3H-imidazo[4,5-b]pyridin-3-yl)benzyl)piperidin-4- | | |
| | yl)amino)pyrimidine-2-carbonitrile | | |
| 507 | (R)-4-((1-(4-(2-(2-aminopyridin-3-yl)-5-(tetrahydrofuran-3- | 573.2 | ++++ |
| | yl)-3H-imidazo[4,5-b]pyridin-3-yl)benzyl)piperidin-4- | | |
| | yl)amino)pyrimidine-2-carbonitrile | | |
| 508 | 4-((1-(4-(2-(2-aminopyridin-3-yl)-5-(hydroxymethyl)-3H- | 533.1 | +++ |
| | imidazo[4,5-b]pyridin-3-yl)benzyl)piperidin-4- | | |
| | yl)amino)pyrimidine-2-carbonitrile | | |
| 509 | 4-((1-(4-(2-(2-aminopyridin-3-yl)-5-(fluoromethyl)-3H- | 535.1 | ++++ |
| | imidazo[4,5-b]pyridin-3-yl)benzyl)piperidin-4- | | |
| | yl)amino)pyrimidine-2-carbonitrile | | |
| 510 | 4-((1-(4-(2-(2-aminopyridin-3-yl)-5-(pyrrolidin-1-yl)-3H- | 572.4 | ++++ |
| | imidazo[4,5-b]pyridin-3-yl)benzyl)piperidin-4- | | |
| | yl)amino)pyrimidine-2-carbonitrile | | |
| 511 | 4-((1-(4-(2-(2-aminopyridin-3-yl)-5-(2-methoxypyridin-4-yl)- | 610.1 | ++++ |
| | 3H-imidazo[4,5-b]pyridin-3-yl)benzyl)piperidin-4- | | |
| | yl)amino)pyrimidine-2-carbonitrile | | |
| 512 | 4-((1-(4-(2-(2-aminopyridin-3-yl)-5-(5-methoxypyridin-3-yl)- | 610.3 | ++++ |
| | 3H-imidazo[4,5-b]pyridin-3-yl)benzyl)piperidin-4- | | |
| | yl)amino)pyrimidine-2-carbonitrile | | |
| 513 | 4-((1-(4-(2-(2-aminopyridin-3-yl)-5-ethynyl-3H-imidazo[4,5- | 527.2 | ++++ |
| | b]pyridin-3-yl)benzyl)piperidin-4-yl)amino)pyrimidine-2- | | |
| | carbonitrile | | |
| 514 | N-(3-(2-(2-aminopyridin-3-yl)-3-(4-((4-((2-cyanopyrimidin-4- | 650.1 | ++++ |
| | yl)amino)piperidin-1-yl)methyl)phenyl)-3H-imidazo[4,5- | | |
| | b]pyridin-5-yl)phenyl)-N-methylacetamide | | |
| 515 | 4-((1-(4-(2-(2-aminopyridin-3-yl)-5-(3-methoxypyridin-4-yl)- | 610.1 | ++++ |
| | 3H-imidazo[4,5-b]pyridin-3-yl)benzyl)piperidin-4- | | |
| | yl)amino)pyrimidine-2-carbonitrile | | |
| 516 | 4-((1-(4-(2-(2-aminopyridin-3-yl)-5-(6-methoxypyridin-3-y])- | 610.2 | ++++ |
| | 3H-imidazo[4,5-b]pyridin-3-yl)benzyl)piperidin-4- | | |
| | yl)amino)pyrimidine-2-carbonitrile | | |
| 517 | 4-((1-(4-(5-(3-aminophenyl)-2-(2-aminopyridin-3-yl)-3H- | 594.3 | ++++ |
| | imidazo[4,5-b]pyridin-3-yl)benzyl)piperidin-4- | | |
| | yl)amino)pyrimidine-2-carbonitrile | | |
| 518 | N-(3-(2-(2-aminopyridin-3-yl)-3-(4-((4-((2-cyanopyrimidin-4- | 664.3 | ++++ |
| | yl)amino)piperidin-1-yl)methyl)phenyl)-3H-imidazo[4,5- | | |
| | b]pyridin-5-yl)phenyl)isobutyramide | | |
| 519 | N-(3-(3-(4-((4-((2-cyanopyrimidin-4-yl)amino)piperidin-1- | 734.3 | +++ |
| | yl)methyl)phenyl)-2-(2-isobutyramidopyridin-3-y])-3H- | | |
| | imidazo[4,5-b]pyridin-5-yl)phenyl)isobutyramide | | |
| 520 | methyl (3-(2-(2-aminopyridin-3-yl)-3-(4-((4-((2- | 652.2 | ++++ |
| | cyanopyrimidin-4-yl)amino)piperidin-1-yl)methyl)phenyl)-3H- | | |
| | imidazo[4,5-b]pyridin-5-yl)phenyl)carbamate | | |
| 521 | 4-((1-(4-(2-(2-aminopyridin-3-yl)-5-(3- | 622.3 | ++++ |
| | (dimethylamino)phenyl)-3H-imidazo[4,5-b]pyridin-3- | | |
| | yl)benzyl)piperidin-4-yl)amino)pyrimidine-2-carbonitrile | | |
| 522 | N-(1-(3-(2-(2-aminopyridin-3-yl)-3-(4-((4-((2-cyanopyrimidin- | 733.4 | +++ |
| | 4-yl)amino)piperidin-1-yl)methyl)phenyl)-3H-imidazo[4,5- | | |
| | b]pyridin-5-yl)phenyl)piperidin-4-yl)-N-metbylacetamide | | |
| 523 | 4-((1-(4-(2-(2-aminopyridin-3-yl)-5-(4-fluoro-2- | 611.4 | +++ |
| | methylphenyl)-3H-imidazo[4,5-b]pyridin-3- | | |
| | yl)benzyl)piperidin-4-yl)amino)pyrimidine-2-carbonitrile | | |
| 524 | 2-(2-(2-aminopyridin-3-yl)-3-(4-((4-((2-cyanopyrimidin-4- | 640.2 | + |
| | yl)amino)piperidin-1-yl)methyl)phenyl)-3H-imidazo[4,5- | | |
| | b]pyridin-5-yl)-5-fluorobenzamide | | |
| 525 | 4-((1-(4-(2-(2-aminopyridin-3-yl)-5-(2-cyano-4-fluorophenyl)- | 622.1 | +++ |
| | 3H-imidazo[4,5-b]pyridin-3-yl)benzyl)piperidin-4- | | |
| | yl)amino)pyrimidine-2-carbonitrile | | |
| 526 | 4-((1-(4-(2-(2-aminopyridin-3-yl)-5-(4-fluoro-2- | 665.1 | + |
| | (trifluoromethyl)phenyl)-3H-imidazo[4,5-b]pyridin-3- | | |
| | yl)benzyl)piperidin-4-yl)amino)pyrimidine-2-carbonitrile | | |
| 527 | 4-((1-(4-(2-(2-aminopyridin-3-yl)-5-(o-tolyl)-3H-imidazo[4,5- | 593.2 | ++++ |
| | b]pyridin-3-yl)benzyl)piperidin-4-yl)amino)pyrimidine-2- | | |
| | carbonitrile | | |
| 528 | 4-((1-(4-(2-(2-aminopyridin-3-yl)-5-(m-tolyl)-3H-imidazo[4,5- | 593.4 | ++++ |
| | b]pyridin-3-yl)benzyl)piperidin-4-yl)amino)pyrimidine-2- | | |
| | carbonitrile | | |
| 529 | 4-((1-(4-(2-(2-aminopyridin-3-yl)-5-(p-tolyl)-3H-imidazo[4,5- | 593.3 | ++++ |
| | b]pyridin-3-yl)benzyl)piperidin-4-yl)amino)pyrimidine-2- | | |
| | carbonitrile | | |
| 530 | 4-((1-(4-(2-(2-aminopyridin-3-yl)-5-(3- | 647.3 | +++ |
| | (trifluoromethyl)phenyl)-3H-imidazo[4,5-b]pyridin-3- | | |
| | yl)benzyl)piperidin-4-yl)amino)pyrimidine-2-carbonitrile | | |
| 531 | isopropyl 2-(2-aminopyridin-3-yl)-3-(4-((4-((2- | 589.3 | ++++ |
| | cyanopyrimidin-4-yl)amino)piperidin-1-yl)methyl)phenyl)-3H- | | |
| | imidazo[4,5-b]pyridine-5-carboxylate | | |
| 532 | 4-((1-(4-(2-(2-aminopyridin-3-yl)-1H-benzo[d]imidazol-1- | 502.3 | ++ |
| | yl)benzyl)piperidin-4-yl)amino)pyrimidine-2-carbonitrile | | |
| 533 | 4-((1-(4-(2-(2-aminopyridin-3-yl)-1H-imidazo[4,5-b]pyridin-1- | 503.4 | ++ |
| | yl)benzyl)piperidin-4-yl)amino)pyrimidine-2-carbonitrile | | |
| 534 | 4-((1-(4-(2-(2-aminopyridin-3-yl)-5-(6-methylpyridin-3-yl)- | 594.1 | ++++ |
| | 3H-imidazo[4,5-b]pyridin-3-yl)benzyl)piperidin-4- | | |
| | yl)amino)pyrimidine-2-carbonitrile | | |
| 535 | 4-((1-(4-(2-(2-aminopyridin-3-yl)-5-(difluoromethoxy)-3H- | 569.3 | ++++ |
| | imidazo[4,5-b]pyridin-3-yl)benzyl)piperidin-4- | | |
| | yl)amino)pyrimidine-2-carbonitrile | | |
| 536 | 4-((1-(4-(2-(2-aminopyridin-3-yl)-5-(4-methoxypyridin-3-yl)- | 610.3 | ++++ |
| | 3H-imidazo[4,5-b]pyridin-3-yl)benzyl)piperidin-4- | | |
| | yl)amino)pyrimidine-2-carbonitrile | | |
| 537 | 4-((1-(4-(2-(2-aminopyridin-3-yl)-5-(2-methoxypyridin-3-yl)- | 610.4 | ++++ |
| | 3H-imidazo[4,5-b]pyridin-3-yl)benzyl)piperidin-4- | | |
| | yl)amino)pyrimidine-2-carbonitrile | | |
| 538 | 4-((1-(4-(2-(2-aminopyridin-3-yl)-5-(2-hydroxypyridin-3-yl)- | 596.1 | +++ |
| | 3H-imidazo[4,5-b]pyridin-3-yl)benzyl)piperidin-4- | | |
| | yl)amino)pyrimidine-2-carbonitrile | | |
| 539 | 4-((1-(4-(2-(2-aminopyridin-3-yl)-5-(1,3-dimethyl-1H-pyrazol- | 597.1 | ++++ |
| | 4-yl)-3H-imidazo[4,5-b]pyridin-3-yl)benzyl)piperidin-4- | | |
| | yl)amino)pyrimidine-2-carbonitrile | | |
| 540 | 4-((1-(4-(2-(2-aminopyridin-3-yl)-1H-imidazo[4,5-c]pyridin-1- | 503.2 | ++ |
| | yl)benzyl)piperidin-4-yl)amino)pyrimidine-2-carbonitrile | | |
| 541 | 4-((1-(4-(2-(2-aminopyridin-3-yl)-5-(4-fluorophenyl)-3H- | 611.3 | ++++ |
| | imidazo[4,5-b]pyridin-3-yl)benzyl)piperidin-4- | | |
| | yl)(methyl)amino)pyrimidine-2-carbonitrile | | |
| 542 | 4-((1-(4-(2-(2-aminopyridin-3-yl)-5-(6- | 646.3 | ++++ |
| | (difluoromethoxy)pyridin-3-yl)-3H-imidazo[4,5-b]pyridin-3- | | |
| | yl)benzyl)piperidin-4-yl)amino)pyrimidine-2-carbonitrile | | |
| 543 | 4-((1-(4-(2-(2-aminopyridin-3-yl)-5-(2- | 646.3 | +++ |
| | (difluoromethoxy)pyridin-4-yl)-3H-imidazo[4,5-b]pyridin-3- | | |
| | yl)benzyl)piperidin-4-yl)amino)pyrimidine-2-carbonitrile | | |
| 544 | 4-((1-(4-(2-(2-aminopyridin-3-yl)-5-(4-fluoro-2- | 613.2 | ++++ |
| | hydroxyphenyl)-3H-imidazo[4,5-b]pyridin-3- | | |
| | yl)benzyl)piperidin-4-yl)amino)pyrimidine-2-carbonitrile | | |
| 545 | 4-((1-(4-(2-(2-aminopyridin-3-yl)-5-(4-fluoro-3- | 613.2 | ++++ |
| | hydroxyphenyl)-3H-imidazo[4,5-b]pyridin-3- | | |
| | yl)benzyl)piperidin-4-yl)amino)pyrimidine-2-carbonitrile | | |
| 546 | 4-((1-(4-(2-(2-aminopyridin-3-yl)-5-(1-methyl-1H-imidazol-5- | 583.3 | ++++ |
| | yl)-3H-imidazo[4,5-b]pyridin-3-yl)benzyl)piperidin-4- | | |
| | yl)amino)pyrimidine-2-carbonitrile | | |
| 547 | 4-((1-(4-(2-(2-aminopyridin-3-yl)-5-(5-methylpyridin-2-y])- | 594.3 | ++++ |
| | 3H-imidazo[4,5-b]pyridin-3-yl)benzyl)piperidin-4- | | |
| | yl)amino)pyrimidine-2-carbonitrile | | |
| 548 | 4-((1-(4-(2-(2-aminopyridin-3-yl)-5-(4-cyanopyridin-2-yl)-3H- | 605.5 | ++++ |
| | imidazo[4,5-b]pyridin-3-yl)benzyl)piperidin-4- | | |
| | yl)amino)pyrimidine-2-carbonitrile | | |
| 549 | 4-((1-(4-(2-(2-aminopyridin-3-yl)-6-bromo-5-(4- | 675.0, | ++++ |
| | fluorophenyl)-3H-imidazo[4,5-b]pyridin-3- | 677.0 | |
| | yl)benzyl)piperidin-4-yl)amino)pyrimidine-2-carbonitrile | | |
| 550 | 2-(2-aminopyridin-3-yl)-3-(4-((4-((2-cyanopyrimidin-4- | 542.2 | ++++ |
| | yl)amino)piperidin-1-yl)methyl)phenyl)-5-methyl-3H- | | |
| | imidazo[4,5-b]pyridine-6-carbonitrile | | |
| 551 | 4-((1-(4-(2-(2-aminopyridin-3-yl)-5-(3-fluoropyridin-4-yl)-3H- | 598.2 | ++++ |
| | imidazo[4,5-b]pyridin-3-yl)benzyl)piperidin-4- | | |
| | yl)amino)pyrimidine-2-carbonitrile | | |
| 552 | 4-((1-(4-(2-(2-aminopyridin-3-yl)-5-(5-methoxypyridin-2-yl)- | 610.2 | ++++ |
| | 3H-imidazo[4,5-b]pyridin-3-yl)benzyl)piperidin-4- | | |
| | yl)amino)pyrimidine-2-carbonitrile | | |
| 553 | 2-(2-aminopyridin-3-yl)-1-(4-((4-((2-cyanopyrimidin-4- | 528.2 | +++ |
| | ylamino)piperidin-1-yl)methyl)phenyl)-1H-imidazo[4,5- | | |
| | c]pyridine-6-carbonitrile | | |
| 554 | 4-((1-(4-(2-(2-aminopyridin-3-yl)-5-(4-methyloxazol-5-yl)- | 584.2 | ++++ |
| | 3H-imidazo[4,5-b]pyridin-3-yl)benzyl)piperidin-4- | | |
| | yl)amino)pyrimidine-2-carbonitrile | | |
| 555 | 4-((1-(4-(2-(2-aminopyridin-3-yl)-5-(1-cyclopropyl-1H- | 609.3 | ++++ |
| | pyrazol-4-yl)-3H-imidazo[4,5-b]pyridin-3-yl)benzyl)piperidin- | | |
| | 4-yl)amino)pyrimidine-2-carbonitrile | | |
| 556 | 2-(2-aminopyridin-3-yl)-1-(4-((4-((2-cyanopyrimidin-4- | 554.2 | + |
| | yl)amino)piperidin-1-yl)methyl)phenyl)-1H-imidazo[4,5- | | |
| | b]pyrazine-5,6-dicarbonitrile | | |
| 557 | 2-(2-aminopyridin-3-yl)-3-(4-((4-((2-cyanopyrimidin-4- | 622.2 | ++++ |
| | yl)amino)piperidin-1-yl)methyl)phenyl)-5-(4-fluorophenyl)- | | |
| | 3H-imidazo[4,5-b]pyridine-6-carbonitrile | | |
| 558 | 4-((1-(4-(2-(2-aminopyridin-3-yl)-5-(2-fluoropyridin-3-yl)-3H- | 598.1 | ++++ |
| | imidazo[4,5-b]pyridin-3-yl)benzyl)piperidin-4- | | |
| | yl)amino)pyrimidine-2-carbonitrile | | |
| 559 | 4-((1-(4-(2-(2-aminopyridin-3-yl)-5-(1-methyl-1H-1,2,4- | 584.2 | +++ |
| | triazol-3-yl)-3H-imidazo[4,5-b]pyridin-3-yl)benzyl)piperidin- | | |
| | 4-yl)amino)pyrimidine-2-carbonitrile | | |
| 560 | 4-((1-(4-(2-(2-aminopyridin-3-yl)-5-(5-ethoxypyridin-2-yl)- | 624.3 | ++++ |
| | 3H-imidazo[4,5-b]pyridin-3-yl)benzyl)piperidin-4- | | |
| | yl)amino)pyrimidine-2-carbonitrile | | |
| 561 | 4-((1-(4-(2-(2-aminopyridin-3-yl)-5-(2-fluoropyridin-4-yl)-3H- | 598.3 | ++++ |
| | imidazo[4,5-b]pyridin-3-yl)benzyl)piperidin-4- | | |
| | yl)amino)pyrimidine-2-carbonitrile | | |
| 562 | 4-((1-(4-(2-(3-aminopyrazin-2-yl)-5-(2-methyl-2H-1,2,3- | 585.2 | ++++ |
| | triazol-4-yl)-3H-imidazo[4,5-b]pyridin-3-yl)benzyl)piperidin- | | |
| | 4-yl)amino)pyrimidine-2-carbonitrile | | |
| 563 | 4-((1-(4-(2-(2-aminopyridin-3-yl)-5-(3-methoxypyridin-2-yl)- | 610.2 | ++++ |
| | 3H-imidazo[4,5-b]pyridin-3-yl)benzyl)piperidin-4- | | |
| | yl)amino)pyrimidine-2-carbonitrile | | |
| 564 | 4-((1-(4-(2-(2-aminopyridin-3-yl)-5-(1-methyl-1H-1,2,3- | 584.2 | +++ |
| | triazol-5-yl)-3H-imidazo[4,5-b]pyridin-3-yl)benzyl)piperidin- | | |
| | 4-yl)amino)pyrimidine-2-carbonitrile | | |
| 565 | 2-(2-aminopyridin-3-yl)-3-(4-((4-((2-cyanopyrimidin-4- | 542.1 | ++++ |
| | yl)amino)piperidin-1-yl)methyl)phenyl)-6-methyl-3H- | | |
| | imidazo[4,5-b]pyridine-5-carbonitrile | | |
| 566 | 4-((1-(4-(2-(2-aminopyridin-3-yl)-5-(5-methyl-1,3,4- | 585.2 | +++ |
| | oxadiazol-2-yl)-3H-imidazo[4,5-b]pyridin-3- | | |
| | yl)benzyl)piperidin-4-yl)amino)pyrimidine-2-carbonitrile | | |
| 567 | 4-((1-(4-(2-(2-aminopyridin-3-yl)-5-(1-methyl-1H-1,2,4- | 584.2 | ++++ |
| | triazol-5-yl)-3H-imidazo[4,5-b]pyridin-3-yl)benzyl)piperidin- | | |
| | 4-yl)amino)pyrimidine-2-carbonitrile | | |
| 568 | 4-((1-(4-(2-(2-aminopyridin-3-yl)-5-(4-methyl-2H-1,2,3- | 584.2 | ++++ |
| | triazol-2-yl)-3H-imidazo[4,5-b]pyridin-3-yl)benzyl)piperidin- | | |
| | 4-yl)amino)pyrimidine-2-carbonitrile | | |
| 569 | 4-((1-(4-(2-(2-aminopyridin-3-yl)-5-(4-methyl-4H-1,2,4- | 584.2 | ++ |
| | triazol-3-yl)-3H-imidazo[4,5-b]pyridin-3-yl)benzyl)piperidin- | | |
| | 4-yl)amino)pyrimidine-2-carbonitrile | | |
| 570 | 4-((1-(4-(2-(2-aminopyridin-3-yl)-5-(3-fluoropyridin-2-yl)-3H- | 620.1 | ++++ |
| | imidazo[4,5-b]pyridin-3-yl)benzyl)piperidin-4- | [M + Na]⁺ | |
| | yl)amino)pyrimidine-2-carbonitrile | | |
| 571 | 4-((1-(4-(2-(2-aminopyridin-3-yl)-5-(6-methylpyridin-2-yl)- | 616.1 | ++++ |
| | 3H-imidazo[4,5-b]pyridin-3-yl)benzyl)piperidin-4- | [M + Na]⁺ | |
| | ylamino)pyrimidine-2-carbonitrile | | |
| 572 | 4-((1-(4-(2-(2-aminopyridin-3-yl)-5-(1-(difluoromethyl)-1H- | 620.1 | ++++ |
| | 1,2,4-triazol-3-yl)-3H-imidazo[4,5-b]pyridin-3- | | |
| | yl)benzyl)piperidin-4-yl)amino)pyrimidine-2-carbonitrile | | |
| 573 | 4-((1-(4-(2-(4-aminopyrimidin-5-yl)-5-(5-fluoropyridin-2-yl)- | 599.2 | +++ |
| | 3H-imidazo[4,5-b]pyridin-3-yl)benzyl)piperidin-4- | | |
| | yl)amino)pyrimidine-2-carbonitrile | | |
| 574 | 4-((1-(4-(2-(3-aminopyrazin-2-yl)-5-(5-fluoropyridin-2-yl)- | 599.0 | ++++ |
| | 3H-imidazo[4,5-b]pyridin-3-yl)benzyl)piperidin-4- | | |
| | yl)amino)pyrimidine-2-carbonitrile | | |
| 575 | 4-((1-(4-(5-(5-fluoropyridin-2-yl)-2-(pyridin-3-yl)-3H- | 583.1 | ++ |
| | imidazo[4,5-b]pyridin-3-yl)benzyl)piperidin-4- | | |
| | yl)amino)pyrimidine-2-carbonitrile | | |
| 576 | 4-((1-(4-(2-(2-aminophenyl)-5-(5-fluoropyridin-2-yl)-3H- | 597.2 | +++ |
| | imidazo[4,5-b]pyridin-3-yl)benzyl)piperidin-4- | | |
| | yl)amino)pyrimidine-2-carbonitrile | | |
| 577 | 4-((1-(4-(5-(5-fluoropyridin-2-yl)-2-phenyl-3H-imidazo[4,5- | 582.2 | +++ |
| | b]pyridin-3-yl)benzyl)piperidin-4-yl)amino)pyrimidine-2- | | |
| | carbonitrile | | |
| 578 | 4-((1-(4-(2-(2-aminopyridin-3-yl)-5-(5-(methoxy-d3)pyridin-2- | 613.2 | ++++ |
| | yl)-3H-imidazo[4,5-b]pyridin-3-yl)benzyl)piperidin-4- | | |
| | yl)amino)pyrimidine-2-carbonitrile | | |
| 579 | 4-((1-(4-(2-(2-aminopyridin-3-yl)-5-(5- | 646.3 | ++++ |
| | (difluoromethoxy)pyridin-2-yl)-3H-imidazo[4,5-b]pyridin-3- | | |
| | yl)benzyl)piperidin-4-yl)amino)pyrimidine-2-carbonitrile | | |
| 580 | 4-((1-(4-(2-(2-aminopyridin-3-yl)-5-(5- | 628.2 | ++++ |
| | (fluoromethoxy)pyridin-2-yl)-3H-imidazo[4,5-b]pyridin-3- | | |
| | yl)benzyl)piperidin-4-yl)amino)pyrimidine-2-carbonitrile | | |
| 581 | 4-((1-(4-(2-(2-aminopyridin-3-yl)-5-(5-methoxypyrimidin-2- | 611.3 | +++ |
| | yl)-3H-imidazo[4,5-b]pyridin-3-yl)benzyl)piperidin-4- | | |
| | yl)amino)pyrimidine-2-carbonitrile | | |
| 582 | 4-((1-(4-(2-(2-aminopyridin-3-yl)-5-(5-methylpyrazin-2-yl)- | 595.3 | ++++ |
| | 3H-imidazo[4,5-b]pyridin-3-yl)benzyl)piperidin-4- | | |
| | yl)amino)pyrimidine-2-carbonitrile | | |
| 583 | 4-((1-(4-(2-(2-aminopyridin-3-yl)-5-(5-methoxypyrazin-2-yl)- | 611.2 | ++++ |
| | 3H-imidazo[4,5-b]pyridin-3-yl)benzyl)piperidin-4- | | |
| | yl)amino)pyrimidine-2-carbonitrile | | |
| 584 | 4-((1-(4-(2-(2-aminopyridin-3-yl)-5-(6-ethoxypyridin-3-yl)- | 624.1 | ++++ |
| | 3H-imidazo[4,5-b]pyridin-3-yl)benzyl)piperidin-4- | | |
| | yl)amino)pyrimidine-2-carbonitrile | | |
| 585 | 4-((1-(4-(2-(2-aminopyridin-3-yl)-5-(5-cyclopropoxypyridin-2- | 658.2 | ++++ |
| | yl)-3H-imidazo[4,5-b]pyridin-3-yl)benzyl)piperidin-4- | [M + Na]⁺ | |
| | yl)amino)pyrimidine-2-carbonitrile | | |
| 586 | 4-((1-(4-(2-(2-aminopyridin-3-yl)-5-(5-isopropoxypyridin-2- | 638.4 | ++++ |
| | yl)-3H-imidazo[4,5-b]pyridin-3-yl)benzyl)piperidin-4- | | |
| | yl)amino)pyrimidine-2-carbonitrile | | |
| 587 | 4-((1-(4-(2-(2-aminopyridin-3-yl)-5-(6-cyclopropylpyridin-3- | 620.3 | ++++ |
| | yl)-3H-imidazo[4,5-b]pyridin-3-yl)benzyl)piperidin-4- | | |
| | yl)amino)pyrimidine-2-carbonitrile | | |
| 588 | 4-((1-(4-(2-(2-aminopyridin-3-yl)-5-(3-methyl-4H-1,2,4- | 584.4 | +++ |
| | triazol-4-yl)-3H-imidazo[4,5-b]pyridin-3-yl)benzyl)piperidin- | | |
| | 4-yl)amino)pyrimidine-2-carbonitrile | | |
| 589 | 4-((1-(4-(2-(2-aminopyridin-3-yl)-5-(3-methyl-1H-1,2,4- | 584.3 | +++ |
| | triazol-1-yl)-3H-imidazo[4,5-b]pyridin-3-yl)benzyl)piperidin- | | |
| | 4-yl)amino)pyrimidine-2-carbonitrile | | |
| 590 | 4-((1-(4-(2-(2-aminopyridin-3-yl)-5-(1H-1,2,4-triazol-1-y])- | 570.2 | ++++ |
| | 3H-imidazo[4,5-b]pyridin-3-yl)benzyl)piperidin-4- | | |
| | yl)amino)pyrimidine-2-carbonitrile | | |
| 591 | 4-((1-(4-(2-(2-aminopyridin-3-yl)-5-(1H-1,2,3-triazol-1-yl)- | 570.1 | ++++ |
| | 3H-imidazo[4,5-b]pyridin-3-yl)benzyl)piperidin-4- | | |
| | yl)amino)pyrimidine-2-carbonitrile | | |
| 592 | 4-((1-(4-(2-(2-aminopyridin-3-yl)-5-(2H-1,2,3-triazol-2-yl)- | 570.2 | ++++ |
| | 3H-imidazo[4,5-b]pyridin-3-yl)benzyl)piperidin-4- | | |
| | yl)amino)pyrimidine-2-carbonitrile | | |
| 593 | 4-((1-(4-(2-(2-aminopyridin-3-yl)-5-(4-methyl-1H-1,2,3- | 584.4 | ++++ |
| | triazol-1-yl)-3H-imidazo[4,5-b]pyridin-3-yl)benzyl)piperidin- | | |
| | 4-yl)amino)pyrimidine-2-carbonitrile | | |
| 594 | 4-((1-(4-(2-(2-aminopyridin-3-yl)-5-(oxazol-4-yl)-3H- | 570.2 | ++++ |
| | imidazo[4,5-b]pyridin-3-yl)benzyl)piperidin-4- | | |
| | yl)amino)pyrimidine-2-carbonitrile | | |
| 595 | 4-((1-(4-(2-(2-aminopyridin-3-yl)-5-(4-fluoropyridin-3-yl)-3H- | 620.2 | ++++ |
| | imidazo[4,5-b]pyridin-3-yl)benzyl)piperidin-4- | [M + Na]⁺ | |
| | yl)amino)pyrimidine-2-carbonitrile | | |
| 596 | 4-((1-(4-(2-(2-aminopyridin-3-yl)-5-(4-methylpyridin-2-yl)- | 616.2 | ++++ |
| | 3H-imidazo[4,5-b]pyridin-3-yl)benzyl)piperidin-4- | [M + Na]⁺ | |
| | yl)amino)pyrimidine-2-carbonitrile | | |
| 597 | 4-((1-(4-(2-(2-aminopyridin-3-yl)-5-(6-(methoxy-d3)pyridin-3- | 613.3 | ++++ |
| | yl-2-d)-3H-imidazo[4,5-b]pyridin-3-yl)benzyl)piperidin-4- | | |
| | yl)amino)pyrimidine-2-carbonitrile | | |
| | Mixture of 3D (57.4% and 4D (38.2%) | | |
| 598 | 4-((1-(4-(2-(2-aminopyridin-3-yl)-5-(6-(methoxy-d3)pyridin-3- | 612.72 | ++++ |
| | yl)-3H-imidazo[4,5-b]pyridin-3-yl)benzyl)piperidin-4- | | |
| | yl)amino)pyrimidine-2-carbonitrile | | |
| 599 | 2-(2-aminopyridin-3-yl)-3-(4-((4-((2-cyanopyrimidin-4- | 544.2 | + |
| | ylamino)piperidin-1-yl)methyl)phenyl)-5-hydroxy-3H- | | |
| | imidazo[4,5-b]pyridine-6-carbonitrile | | |
| 600 | 4-((1-(4-(2-(2-amino-4-fluoropyridin-3-yl)-5-(5-fluoropyridin- | 616.2 | +++ |
| | 2-yl)-3H-imidazo[4,5-b]pyridin-3-yl)benzyl)piperidin-4- | | |
| | yl)amino)pyrimidine-2-carbonitrile | | |
| 601 | 4-((1-(4-(5-(5-fluoropyridin-2-yl)-2-(pyrimidin-5-yl)-3H- | 584.2 | + |
| | imidazo[4,5-b]pyridin-3-yl)benzyl)piperidin-4- | | |
| | yl)amino)pyrimidine-2-carbonitrile | | |
| 602 | 4-((1-(4-(2-(2-aminopyridin-3-yl)-6-(5-fluoropyridin-2-yl)-1H- | 596.67 | ++++ |
| | benzo[d]imidazol-1-yl)benzyl)piperidin-4- | | |
| | yl)amino)pyrimidine-2-carbonitrile | | |
| 603 | 4-((1-(4-(2-(2-aminopyridin-3-yl)-5-(5-fluoropyrimidin-2-y])- | 599.1 | ++++ |
| | 3H-imidazo[4,5-b]pyridin-3-yl)benzyl)piperidin-4- | | |
| | yl)amino)pyrimidine-2-carbonitrile | | |
| 604 | 4-((1-(4-(2-(2-aminopyridin-3-yl)-5-(5-methylpyrimidin-2-yl)- | 595.2 | ++++ |
| | 3H-imidazo[4,5-b]pyridin-3-yl)benzyl)piperidin-4- | | |
| | yl)amino)pyrimidine-2-carbonitrile | | |
| 605 | 4-((1-(4-(2-(2-aminopyridin-3-yl)-5-(5-fluoropyrazin-2-yl)- | 599.3 | ++++ |
| | 3H-imidazo[4,5-b]pyridin-3-yl)benzyl)piperidin-4- | | |
| | yl)amino)pyrimidine-2-carbonitrile | | |
| 606 | 4-((1-(4-(2-(2-aminopyridin-3-yl)-5-(6- | 628.2 | ++++ |
| | (fluoromethoxy)pyridin-3-yl)-3H-imidazo[4,5-b]pyridin-3- | | |
| | yl)benzyl)piperidin-4-yl)amino)pyrimidine-2-carbonitrile | | |
| 607 | 4-((1-(4-(2-(2-aminopyridin-3-yl)-5-(1-(fluoromethyl)-6-oxo- | 628.2 | +++ |
| | 1,6-dihydropyridin-3-yl)-3H-imidazo[4,5-b]pyridin-3- | | |
| | yl)benzyl)piperidin-4-yl)amino)pyrimidine-2-carbonitrile | | |
| 608 | 4-((1-(4-(2-(2-aminopyridin-3-yl)-5-(2-(methyl-d3)-2H-1,2,3- | 587.2 | ++++ |
| | triazol-4-yl)-3H-imidazo[4,5-b]pyridin-3-yl)benzyl)piperidin- | | |
| | 4-yl)amino)pyrimidine-2-carbonitrile | | |
| 609 | 4-((1-(4-(2-(2-aminopyridin-3-yl)-5-(2-ethyl-2H-1,2,3-triazol- | 620.2 | ++++ |
| | 4-yl)-3H-imidazo[4,5-b]pyridin-3-yl)benzyl)piperidin-4- | [M + Na]⁺ | |
| | ylamino)pyrimidine-2-carbonitrile | | |
| 610 | 4-((1-(4-(2-(2-aminopyridin-3-yl)-5-(2-isopropyl-2H-1,2,3- | 612.3 | ++++ |
| | triazol-4-yl)-3H-imidazo[4,5-b]pyridin-3-yl)benzyl)piperidin- | | |
| | 4-yl)amino)pyrimidine-2-carbonitrile | | |
| 611 | 4-((1-(4-(2-(2-aminopyridin-3-yl)-5-(1-isopropyl-1H-1,2,3- | 612.3 | ++++ |
| | triazol-4-yl)-3H-imidazo[4,5-b]pyridin-3-yl)benzyl)piperidin- | | |
| | 4-yl)amino)pyrimidine-2-carbonitrile | | |
| 612 | 4-((1-(4-(2-(2-aminopyridin-3-yl)-5-(2-methyl-5-(methyl-d3)- | 600.71 | ++++ |
| | 2H-1,2,3-triazol-4-yl)-3H-imidazo[4,5-b]pyridin-3- | | |
| | yl)benzyl)piperidin-4-yl)amino)pyrimidine-2-carbonitrile | | |
| 613 | 4-((1-(4-(2-(2-aminopyridin-3-yl)-5-(2H-1,2,3-triazol-4-yl)- | 570.3 | ++++ |
| | 3H-imidazo[4,5-b]pyridin-3-yl)benzyl)piperidin-4- | | |
| | yl)amino)pyrimidine-2-carbonitrile | | |
| 614 | 4-((1-(4-(2-(2-aminopyridin-3-yl)-5-(2,5-dimethyl-2H-1,2,3- | 597.69 | ++++ |
| | triazol-4-yl)-3H-imidazo[4,5-b]pyridin-3-yl)benzyl)piperidin- | | |
| | 4-yl)amino)pyrimidine-2-carbonitrile | | |
| 615 | 4-((1-(4-(2-(2-aminopyridin-3-yl)-5-(2-cyclopropyl-2H-1,2,3- | 610.4 | ++++ |
| | triazol-4-yl)-3H-imidazo[4,5-b]pyridin-3-yl)benzyl)piperidin- | | |
| | 4-yl)amino)pyrimidine-2-carbonitrile | | |
| 616 | 4-((1-(4-(2-(2-aminopyridin-3-yl)-5-(2-methyl-2H-1,2,3- | 585.2 | ++++ |
| | triazol-4-yl-5-d)-3H-imidazo[4,5-b]pyridin-3- | | |
| | yl)benzyl)piperidin-4-yl)amino)pyrimidine-2-carbonitrile | | |
| 617 | 4-((1-(4-(2-(2-aminopyridin-3-yl)-5-(1-(difluoromethyl)-6- | 646.0 | +++ |
| | 0x0-1,6-dihydropyridin-3-yl)-3H-imidazo[4,5-b]pyridin-3- | | |
| | yl)benzyl)piperidin-4-yl)amino)pyrimidine-2-carbonitrile | | |
| 618 | 4-((1-(4-(2-(2-aminopyridin-3-yl)-5-(2-(fluoromethyl)-2H- | 602.3 | ++++ |
| | 1,2,3-triazol-4-yl)-3H-imidazo[4,5-b]pyridin-3- | | |
| | yl)benzyl)piperidin-4-yl)amino)pyrimidine-2-carbonitrile | | |
| 619 | 4-((1-(4-(2-(2-aminopyridin-3-yl)-5-(1-(fluoromethyl)-1H- | 602.2 | ++++ |
| | 1,2,3-triazol-4-yl)-3H-imidazo[4,5-b]pyridin-3- | | |
| | yl)benzyl)piperidin-4-yl)amino)pyrimidine-2-carbonitrile | | |
| 620 | 4-((1-(4-(2-(2-aminopyridin-3-yl)-5-(2-(difluoromethyl)-2H- | 620.2 | ++++ |
| | 1,2,3-triazol-4-yl)-3H-imidazo[4,5-b]pyridin-3- | | |
| | yl)benzyl)piperidin-4-yl)amino)pyrimidine-2-carbonitrile | | |
| 621 | 4-((1-(4-(2-(3-aminopyrazin-2-yl)-5-(2-(methyl-d3)-2H-1,2,3- | 588.1 | ++++ |
| | triazol-4-yl)-3H-imidazo[4,5-b]pyridin-3-yl)benzyl)piperidin- | | |
| | 4-yl)amino)pyrimidine-2-carbonitrile | | |
| 622 | 4-((1-(4-(2-(3-aminoisoxazol-4-yl)-5-(5-fluoropyridin-2-yl)- | 588.4 | ++ |
| | 3H-imidazo[4,5-b]pyridin-3-yl)benzyl)piperidin-4- | | |
| | yl)amino)pyrimidine-2-carbonitrile | | |
| 623 | 4-((1-(4-(2-(2-aminopyridin-3-yl)-5-(6-(fluoromethoxy- | 630.2 | ++++ |
| | d2)pyridin-3-yl)-3H-imidazo[4,5-b]pyridin-3- | | |
| | yl)benzyl)piperidin-4-yl)amino)pyrimidine-2-carbonitrile | | |
| 624 | 4-((1-(4-(2-(2-aminopyridin-3-yl)-5-(1-(fluoromethyl-d2)-6- | 630.2 | +++ |
| | oxo-1,6-dihydropyridin-3-yl)-3H-imidazo[4,5-b]pyridin-3- | | |
| | yl)benzyl)piperidin-4-yl)amino)pyrimidine-2-carbonitrile | | |
| 625 | 4-((1-(4-(2-(2-aminopyridin-3-yl)-5-(1-ethyl-1H-1,2,3-triazol- | 598.2 | ++++ |
| | 4-yl)-3H-imidazo[4,5-b]pyridin-3-yl)benzyl)piperidin-4- | | |
| | yl)amino)pyrimidine-2-carbonitrile | | |
| 626 | 4-((1-(4-(2-(2-aminopyridin-3-yl)-5-(2-(cyanomethyl)-2H- | 609.4 | ++++ |
| | 1,2,3-triazol-4-yl)-3H-imidazo[4,5-b]pyridin-3- | | |
| | yl)benzyl)piperidin-4-yl)amino)pyrimidine-2-carbonitrile | | |
| 627 | 4-((1-(4-(2-(3-aminopyrazin-2-yl)-5-(2-(fluoromethyl)-2H- | 603.2 | ++++ |
| | 1,2,3-triazol-4-yl)-3H-imidazo[4,5-b]pyridin-3- | | |
| | yl)benzyl)piperidin-4-yl)amino)pyrimidine-2-carbonitrile | | |
| 628 | 4-((1-(4-(2-(3-aminopyrazin-2-yl)-5-(1-(fluoromethyl)-1H- | 603.3 | ++++ |
| | 1,2,3-triazol-4-yl)-3H-imidazo[4,5-b]pyridin-3- | | |
| | yl)benzyl)piperidin-4-yl)amino)pyrimidine-2-carbonitrile | | |
| 629 | 4-((1-(4-(2-(3-aminopyrazin-2-yl)-5-(2-ethyl-2H-1,2,3-triazol- | 621.1 | ++++ |
| | 4-yl)-3H-imidazo[4,5-b]pyridin-3-yl)benzyl)piperidin-4- | [M + Na]⁺ | |
| | yl)amino)pyrimidine-2-carbonitrile | | |
| 630 | 4-((1-(4-(2-(3-aminopyrazin-2-yl)-5-(2-isopropyl-2H-1,2,3- | 613.3 | ++++ |
| | triazol-4-yl)-3H-imidazo[4,5-b]pyridin-3-yl)benzyl)piperidin- | | |
| | 4-yl)amino)pyrimidine-2-carbonitrile | | |
| 631 | 4-((1-(4-(2-(3-aminopyrazin-2-yl)-5-(2-cyclopropyl-2H-1,2,3- | 611.1 | ++++ |
| | triazol-4-yl)-3H-imidazo[4,5-b]pyridin-3-yl)benzyl)piperidin- | | |
| | 4-yl)amino)pyrimidine-2-carbonitrile | | |
| 632 | 4-((1-(4-(2-(3-aminopyrazin-2-yl)-5-(2-(cyanomethyl)-2H- | 610.3 | ++++ |
| | 1,2,3-triazol-4-yl)-3H-imidazo[4,5-b]pyridin-3- | | |
| | yl)benzyl)piperidin-4-yl)amino)pyrimidine-2-carbonitrile | | |
| 633 | 2-(4-(2-(3-aminopyrazin-2-yl)-3-(4-((4-((2-cyanopyrimidin-4- | 628.4 | +++ |
| | yl)amino)piperidin-1-yl)methyl)phenyl)-3H-imidazo[4,5- | | |
| | b]pyridin-5-yl)-2H-1,2,3-triazol-2-yl)acetamide | | |
| 634 | 4-((1-(4-(2-(3-aminopyrazin-2-yl)-5-(2-methyl-2H-1,2,3- | 586.3 | ++++ |
| | triazol-4-yl-5-d)-3H-imidazo[4,5-b]pyridin-3- | | |
| | yl)benzyl)piperidin-4-yl)amino)pyrimidine-2-carbonitrile | | |
| 635 | 4-((1-(4-(2-(3-aminopyrazin-2-yl)-5-(2,5-dimethyl-2H-1,2,3- | 599.3 | +++ |
| | triazol-4-yl)-3H-imidazo[4,5-b]pyridin-3-yl)benzyl)piperidin- | | |
| | 4-yl)amino)pyrimidine-2-carbonitrile | | |
| 636 | 4-((1-(4-(2-(3-aminopyrazin-2-yl)-5-(6-methoxypyridin-3-yl)- | 611.2 | ++++ |
| | 3H-imidazo[4,5-b]pyridin-3-yl)benzyl)piperidin-4- | | |
| | yl)amino)pyrimidine-2-carbonitrile | | |
| 637 | 4-((1-(4-(2-(3-aminopyrazin-2-yl)-5-(6-(methoxy-d3)pyridin- | 614.3 | ++++ |
| | 3-yl)-3H-imidazo[4,5-b]pyridin-3-yl)benzyl)piperidin-4- | | |
| | yl)amino)pyrimidine-2-carbonitrile | | |
| 638 | 4-((1-(4-(2-(3-aminopyrazin-2-yl)-5-(5-(methoxy-d3)pyridin- | 614.3 | ++++ |
| | 2-yl)-3H-imidazo[4,5-b]pyridin-3-yl)benzyl)piperidin-4- | | |
| | yl)amino)pyrimidine-2-carbonitrile | | |
| 639 | 4-((1-(4-(2-(3-aminopyrazin-2-yl)-5-(6- | 629.2 | ++++ |
| | (fluoromethoxy)pyridin-3-yl)-3H-imidazo[4,5-b]pyridin-3- | | |
| | yl)benzyl)piperidin-4-yl)amino)pyrimidine-2-carbonitrile | | |
| 640 | 4-((1-(4-(2-(3-aminopyrazin-2-yl)-5-(6-(fluoromethoxy- | 631.2 | ++++ |
| | d2)pyridin-3-yl)-3H-imidazo[4,5-b]pyridin-3- | | |
| | yl)benzyl)piperidin-4-yl)amino)pyrimidine-2-carbonitrile | | |
| 641 | 4-((1-(4-(2-(2-aminopyridin-3-yl)-5-(6-cyclopropoxypyridin-3- | 658.2 | ++++ |
| | yl)-3H-imidazo[4,5-b]pyridin-3-yl)benzyl)piperidin-4- | [M + Na]⁺ | |
| | yl)amino)pyrimidine-2-carbonitrile | | |
| 642 | 4-((1-(4-(2-(2-aminopyridin-3-yl)-5-(6-isopropoxypyridin-3- | 660.2 | ++++ |
| | yl)-3H-imidazo[4,5-b]pyridin-3-yl)benzyl)piperidin-4- | [M + Na]⁺ | |
| | yl)amino)pyrimidine-2-carbonitrile | | |
| 643 | 4-((1-(4-(2-(2-aminopyridin-3-yl)-5-(6-isopropylpyridin-3-yl)- | 622.3 | ++++ |
| | 3H-imidazo[4,5-b]pyridin-3-yl)benzyl)piperidin-4- | | |
| | yl)amino)pyrimidine-2-carbonitrile | | |
| 644 | 4-((1-(4-(2-(2-aminopyridin-3-yl)-5-(5-cyclopropylpyridin-2- | 620.3 | ++++ |
| | yl)-3H-imidazo[4,5-b]pyridin-3-yl)benzyl)piperidin-4- | | |
| | yl)amino)pyrimidine-2-carbonitrile | | |
| 645 | 4-((1-(4-(2-(2-aminopyridin-3-yl)-5-(6- | 630.2 | ++++ |
| | (difluoromethyl)pyridin-3-yl)-3H-imidazo[4,5-b]pyridin-3- | | |
| | yl)benzyl)piperidin-4-yl)amino)pyrimidine-2-carbonitrile | | |
| 646 | 4-((1-(4-(2-(2-aminopyridin-3-yl)-5-(6- | 648.2 | ++++ |
| | (trifluoromethyl)pyridin-3-yl)-3H-imidazo[4,5-b]pyridin-3- | | |
| | yl)benzyl)piperidin-4-yl)amino)pyrimidine-2-carbonitrile | | |
| 647 | 4-((1-(4-(2-(2-aminopyridin-3-yl)-5-(5-ethylpyridin-2-yl)-3H- | 608.3 | ++++ |
| | imidazo[4,5-b]pyridin-3-yl)benzyl)piperidin-4- | | |
| | yl)amino)pyrimidine-2-carbonitrile | | |
| 648 | 4-((1-(4-(2-(2-aminopyridin-3-yl)-5-(5-(fluoromethyl)pyridin- | 612.3 | ++++ |
| | 2-yl)-3H-imidazo[4,5-b]pyridin-3-yl)benzyl)piperidin-4- | | |
| | yl)amino)pyrimidine-2-carbonitrile | | |
| 649 | 4-((1-(4-(2-(2-aminopyridin-3-yl)-5-(5- | 630.3 | ++++ |
| | (difluoromethyl)pyridin-2-yl)-3H-imidazo[4,5-b]pyridin-3- | | |
| | yl)benzyl)piperidin-4-yl)amino)pyrimidine-2-carbonitrile | | |
| 650 | 4-((1-(4-(2-(2-aminopyridin-3-yl)-5-(5- | 648.6 | +++ |
| | (trifluoromethyl)pyridin-2-yl)-3H-imidazo[4,5-b]pyridin-3- | | |
| | yl)benzyl)piperidin-4-yl)amino)pyrimidine-2-carbonitrile | | |

### Example 1; Oral capsule

The active ingredient is a compound of Table 1, or a pharmaceutically acceptable salt or solvate thereof. A capsule for oral administration is prepared by mixing 1-1000 mg of active ingredient with starch or other suitable powder blend. The mixture is incorporated into an oral dosage unit such as a hard gelatin capsule, which is suitable for oral administration.

### Example 2: Solution for injection

The active ingredient is a compound of Table 1, or a pharmaceutically acceptable salt or solvate thereof, and is formulated as a solution in sesame oil at a concentration of 50 mg-eq/mL.

The examples and embodiments described herein are for illustrative purposes only and various modifications or changes suggested to persons skilled in the art are to be included within the spirit and purview of this application and scope of the appended claims.

1.-62. (canceled)

63. A compound having the structure of Formula (Ib), or a pharmaceutically acceptable salt or solvate thereof: 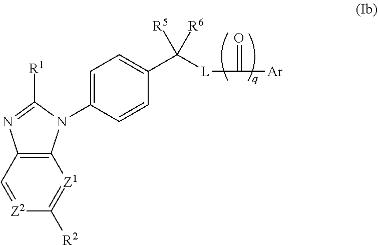
wherein:
Z¹ is N, C—H, or C—R³;
Z² is N, C—H, or C—R⁴;
Ar is selected from: 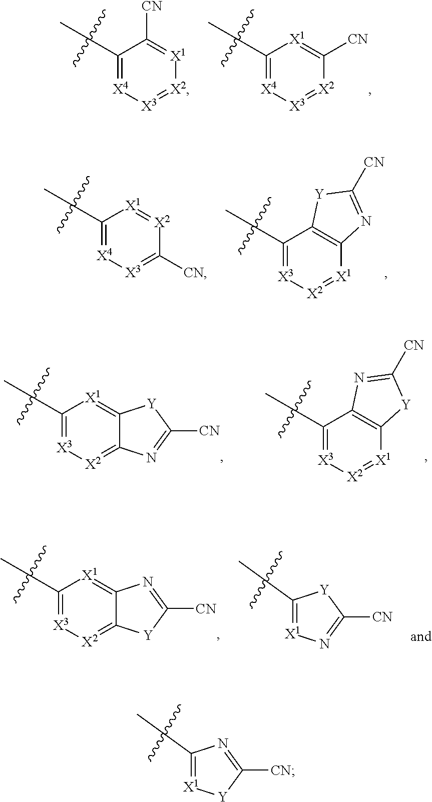
X¹ is N or C—R⁷;
X² is N or C—R⁷;
X³ is N or C—R⁷;
X⁴ is N or C—R⁷;
Y is O, S, or N—R⁹;
R¹ is selected from optionally substituted aryl, or optionally substituted heteroaryl;
R² is selected from hydrogen, optionally substituted C1-C6 alkyl, optionally substituted aryl, or optionally substituted heteroaryl;
R³ is selected from optionally substituted C1-C6 alkyl, or optionally substituted aryl;
R⁴ is selected from optionally substituted C1-C6 alkyl, or optionally substituted aryl;
R⁵ and R⁶ are each independently hydrogen, deuterium, halogen, —OH, or optionally substituted C1-C6 alkyl; or R⁵ and R⁶ together form an oxo; or R⁵ and R⁶ join together to form a carbocycle or heterocycle;
each R⁷ is independently selected from hydrogen, deuterium, halogen, —OH, —SH, optionally substituted C1-C6 alkoxy, —S— (optionally substituted C1-C6 alkyl), —CN, optionally substituted C1-C6 alkyl, and optionally substituted aryl;
L is a divalent radical selected from: 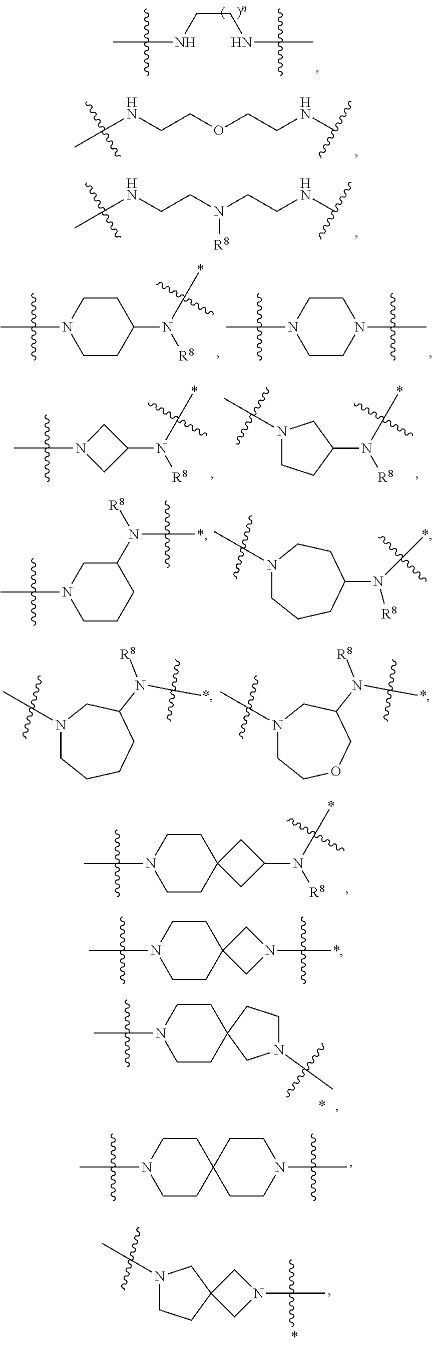 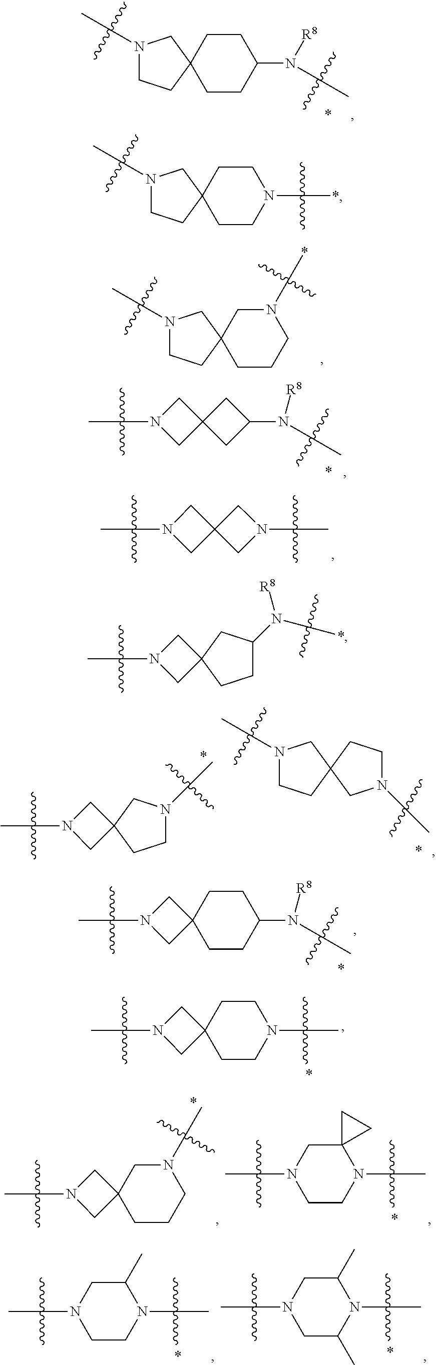 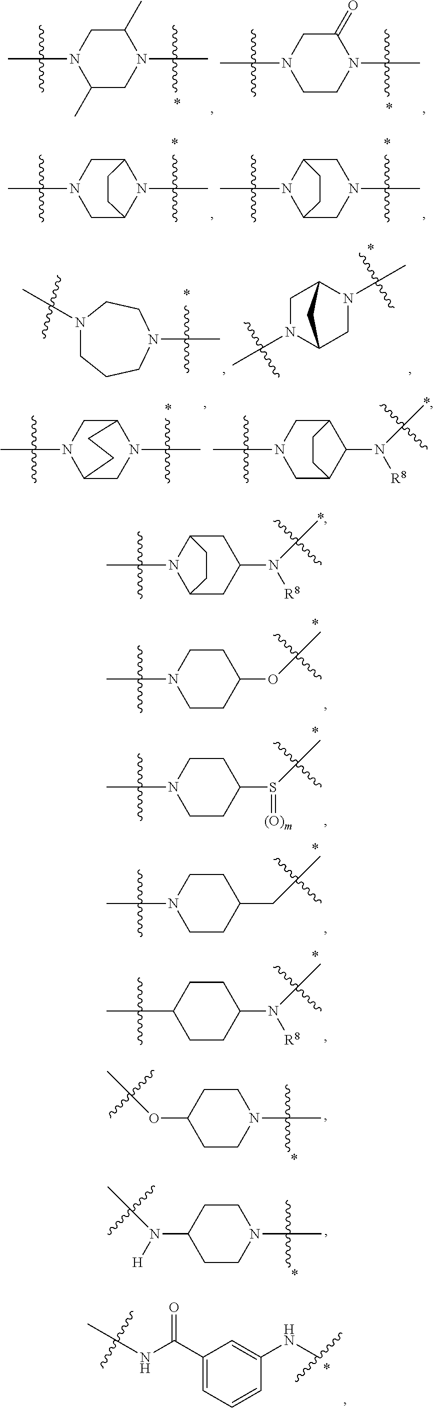 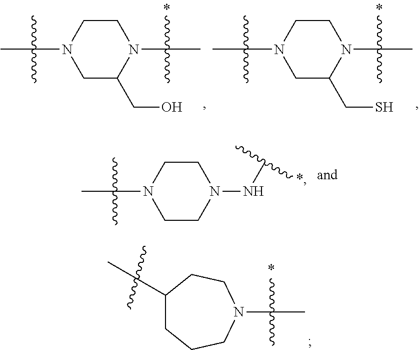 [SMILES: *N1CCN(*)C(CO)C1 ; *N1CCN(*)C(CS)C1 ; *NN1CCN(*)CC1 ; *C1CCCN(*)CC1]
wherein the asterisk (*) indicates the bond to the —(C(O))n-Ar group;
R⁸ is hydrogen, optionally substituted C1-C6 alkyl, optionally substituted C3-C7 cycloalkyl, or optionally substituted heterocyclyl;
R⁹ is hydrogen, or optionally substituted C1-C6 alkyl,
m is 0, 1, or 2;
n is 1, 2, or 3, and
q is 0 or 1.

64. The compound of claim 63, or pharmaceutically acceptable salt or solvate thereof, wherein Z¹ is N.

65. The compound of claim 63, or pharmaceutically acceptable salt or solvate thereof, wherein Z² is C—H.

66. The compound of claim 63, or pharmaceutically acceptable salt or solvate thereof, wherein R¹ is optionally substituted heteroaryl.

67. The compound of claim 66, or pharmaceutically acceptable salt or solvate thereof, wherein the optionally substituted heteroaryl is an optionally substituted pyridyl.

68. The compound of claim 63, or pharmaceutically acceptable salt or solvate thereof, wherein R² is optionally substituted heteroaryl.

69. The compound of claim 68, or pharmaceutically acceptable salt or solvate thereof, wherein R² is optionally substituted pyridyl.

70. The compound of claim 63, or pharmaceutically acceptable salt or solvate thereof, wherein R⁵ and R⁶ are hydrogen.

71. The compound of claim 63, or pharmaceutically acceptable salt or solvate thereof, wherein R⁵ and R⁶ together form an oxo.

72. (canceled)

73. The compound of claim 63, or pharmaceutically acceptable salt or solvate thereof, wherein L is selected from:

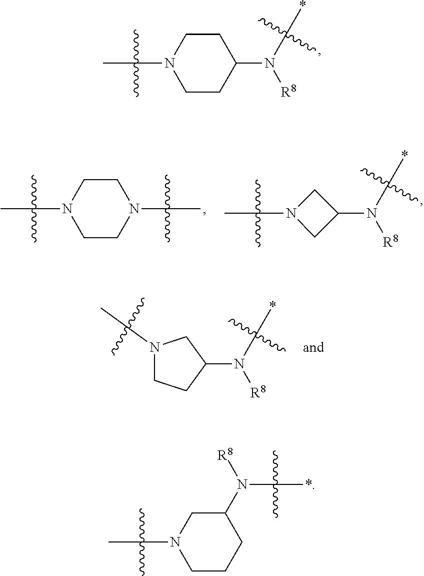

74. The compound of claim 63, or pharmaceutically acceptable salt or solvate thereof, wherein L is selected from:

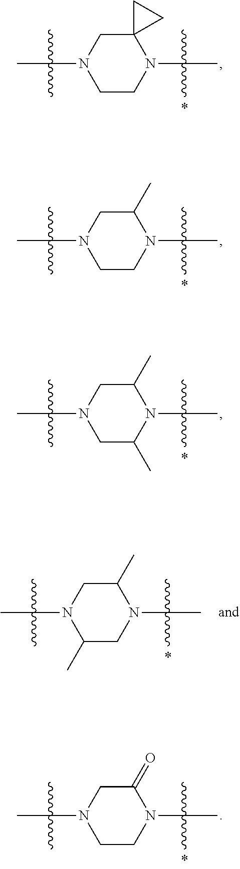

75. The compound of claim 63, or pharmaceutically acceptable salt or solvate thereof, wherein L is selected from:

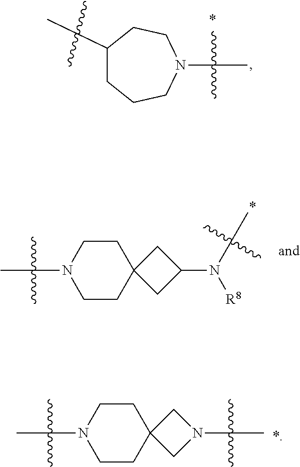

76. The compound of claim 63, or pharmaceutically acceptable salt or solvate thereof, wherein L is selected from:

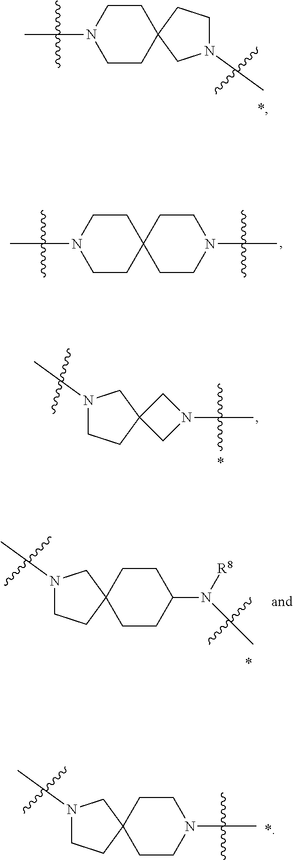

77. The compound of claim 63, or pharmaceutically acceptable salt or solvate thereof, wherein L is selected from:

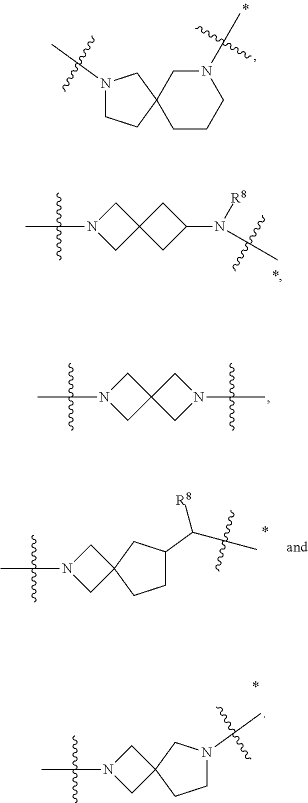

78. The compound of claim 63, or pharmaceutically acceptable salt or solvate thereof, wherein R⁸ is hydrogen or optionally substituted C1-C6 alkyl.

79. The compound of claim 63, or pharmaceutically acceptable salt or solvate thereof, wherein q is 0.

80. The compound of claim 63, or pharmaceutically acceptable salt or solvate thereof, wherein q is 1.

81. The compound of claim 63, or pharmaceutically acceptable salt or solvate thereof, wherein Ar is

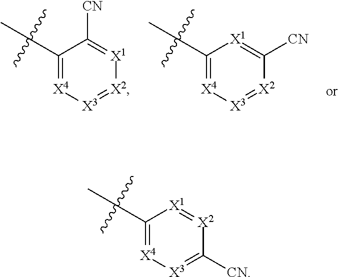

82. The compound of claim 81, or pharmaceutically acceptable salt or solvate thereof, wherein X¹ is N.

83. The compound of claim 81, or pharmaceutically acceptable salt or solvate thereof, wherein X² is N.

84. The compound of claim 81, or pharmaceutically acceptable salt or solvate thereof, wherein X³ is C—H and X⁴ is C—H.

85. The compound of claim 63, or pharmaceutically acceptable salt or solvate thereof, wherein Z¹ is N; Z² is C—H; R¹ is optionally substituted pyridyl; and R² is optionally substituted pyridyl.

86. The compound of claim 63, or pharmaceutically acceptable salt or solvate thereof, wherein q is 0; and R⁵ and R⁶ are hydrogen.

87. The compound of claim 85, or pharmaceutically acceptable salt or solvate thereof, wherein q is 0; and R⁵ and R⁶ are hydrogen.

88. The compound of claim 81, or pharmaceutically acceptable salt or solvate thereof, wherein X¹ is N; X² is N; X³ is C—H; and X⁴ is C—H.

89. The compound of claim 85, or pharmaceutically acceptable salt or solvate thereof, wherein X¹ is N; X² is N; X³ is C—H; and X⁴ is C—H.

90. The compound of claim 86, or pharmaceutically acceptable salt or solvate thereof, wherein X¹ is N; X² is N; X³ is C—H; and X⁴ is C—H.

91. The compound of claim 87, or pharmaceutically acceptable salt or solvate thereof, wherein X¹ is N; X² is N; X³ is C—H; and X⁴ is C—H.

92. A pharmaceutical composition comprising a compound, or pharmaceutically acceptable salt or solvate thereof, as described in claim 63, and a pharmaceutically acceptable excipient.


SEQUENCE LISTING (1 sequence)

SEQ ID NO: 1 - protein, 480 aa, Homo sapiens
MSDVAIVKEGWLHKRGKYIKTWRPRYFLLKNDGTFIGYKERPQDVDQREAPLNNFSVAQCQLMKTERPRPNTFIIRCLQWTTVIERTFHVETPEEREEWTTAIQTVADGLKKQEEEEMDFRSGSPSDNSGAEEMEVSLAKPKHRVTMNEFEYLKLLGKGTFGKVILVKEKATGRYYAMKILKKEVIVAKDEVAHTLTENRVLQNSRHPFLTALKYSFQTHDRLCFVMEYANGGELFFHLSRERVFSEDRARFYGAEIVSALDYLHSEKNVVYRDLKLENLMLDKDGHIKITDFGLCKEGIKDGATMKTFCGTPEYLAPEVLEDNDYGRAVDWWGLGVVMYEMMCGRLPFYNQDHEKLFELILMEEIRFPRTLGPEAKSLLSGLLKKDPKQRLGGGSEDAKEIMQHRFFAGIVWQHVYEKKLSPPFKPQVTSETDTRYFDEEFTAQMITITPPDQDDSMECVDSERRPHFPQFSYSASGTA